(19) United States
(12) Patent Application Publication
(10) Pub. No.: US 2007/0015693 A1
(43) Pub. Date: Jan. 18, 2007

(54) Cyclosporin compositions

(75) Inventors: James N. Chang, Newport Beach, CA (US); Orest Olejnik, Coto De Caza, CA (US); Bruce A. Firestone, Irvine, CA (US)

Correspondence Address: ALLERGAN, INC., 2525 DUPONT DRIVE, T2-7H, IRVINE, CA, 92612-1599, US

(73) Assignee: Allergan, Inc.

(21) Appl. No.: 11/181,509

(22) Filed: Jul. 13, 2005

(51) Int. Cl. A61K 38/13; A61K 36/899; A61K 36/31; A61K 36/48; A61K 36/55

(52) U.S. Cl. 514/11; 424/750; 424/768; 424/755; 424/757; 424/776

(57) ABSTRACT

Cyclosporin A compositions are disclosed herein comprising an oil and a surfactant.

### DESCRIPTION OF THE INVENTION

Disclosed herein is a composition comprising cyclosporin A at a concentration from about 0.001% to about 0.4%, an oil, and a surfactant, wherein said composition is an ophthalmically acceptable emulsion.

Another embodiment comprises from about 0.001% to about 0.1% cyclosporin A.

Another embodiment comprises from about 0.005% to about 0.05% cyclosporin A.

Another embodiment comprises about 0.05% cyclosporin A.

These compositions are useful for, among other things, the treatment of dry eye disease.

One embodiment comprises an oil having a specific gravity from 0.95 to 1.07.

Another embodiment comprises Anise oil.

Another embodiment comprises Castor oil.

Another embodiment comprises Clove oil.

Another embodiment comprises Cassia oil.

Another embodiment comprises Cinnamon oil.

Another embodiment comprises an oil having a specific gravity between 0.90 and 0.95.

Another embodiment comprises Almond oil.

Another embodiment comprises Corn oil.

Another embodiment comprises Arachis oil.

Another embodiment comprises Cottonseed oil.

Another embodiment comprises Safflower oil.

Another embodiment comprises Maize oil.

Another embodiment comprises Linseed oil.

Another embodiment comprises Rapeseed oil.

Another embodiment comprises Soybean oil.

Another embodiment comprises Olive oil.

Another embodiment comprises Caraway oil.

Another embodiment comprises Rosemary oil.

Another embodiment comprises Peanut oil.

Another embodiment comprises Peppermint oil.

Another embodiment comprises Sunflower oil.

Another embodiment comprises Eucalyptus oil.

Another embodiment comprises Sesame oil.

Another embodiment comprises an oil having a specific gravity specific gravity below 0.9.

Another embodiment comprises Mineral oil.

Another embodiment comprises Coriander oil.

Another embodiment comprises Lavender oil.

Another embodiment comprises Citronella oil.

Another embodiment comprises Juniper oil.

Another embodiment comprises Lemon oil.

Another embodiment comprises Orange oil.

Another embodiment comprises Clary sage oil.

Another embodiment comprises Nutmeg oil.

Another embodiment comprises Tea tree oil.

Useful surfactants include, but are not limited to,

Alcohols, including but not limited to,

Diglycerol®, which is available from Solvay Chemicals, Inc.;

Hetoxide GT-80®, which is available from Global-Seven, Inc.;

Lexemul BEO®, which is available from Inolex Chemical Co.;

Polyglycerol-3®, which is available from Solvay Chemicals, Inc.;

Redicote E Series®, which is available from Akzo Nobel Surface Chemistry LLC;

Simulsol OX 1005L®, which is available from Seppic Inc.;

Stanfax 567®, which is available from Para-Chem Standard Div.;

TA-1618®, which is available from Procter & Gamble; and

Witconol H-31A®, which is available from Akzo Nobel Surface Chemistry LLC;

Amine Oxides, including but not limited to,

AO-405®, which is available from Tomah Products®, Inc.;

AO-455®, which is available from Tomah Products®, Inc.;

AO 728 Special®, which is available from Tomah Products®, Inc.;

Barlox 12®, which is available from Lonza Inc.;

Barlox 14®, which is available from Lonza Inc.;

Burcoxide Lo®, which is available from Burlington Chemical Co.®, Inc.;

Caloxamine LO®, which is available from Pilot Chemical Co.;

Chemoxide CAW®, which is available from Chemron Corp.;

Chemoxide LM-30®, which is available from Chemron Corp.;

Chemoxide LO®, which is available from Chemron Corp.;

Chemoxide MO®, which is available from Chemron Corp.;

Colalux CAO-35®, which is available from Colonial Chemical Co.;

Colalux LO®, which is available from Colonial Chemical Co.;

DeMox CAPO®, which is available from DeForest Enterprises, Inc.;

DeMox CSG-30®, which is available from DeForest Enterprises, Inc.;

DeMox LAO®, which is available from DeForest Enterprises, Inc.;

Emcol L®, which is available from Crompton Corp.;

Empigen OB®, which is available from Huntsman LLC;

Empigen OS/A®, which is available from Huntsman LLC;

Foamox CDO®, which is available from Alzo International, Inc.;

Foamox DMM®, which is available from Alzo International, Inc.;

Foamox DMS®, which is available from Alzo International, Inc.;

Genaminox KC®, which is available from Clariant Corporation;

Genaminox LA®, which is available from Clariant Corporation;

Hartofoam SAO®, which is available from Hart Chemical Corp.;

Hartox DMCD®, which is available from Hart Chemical Corp.;

Lipowax DAT®, which is available from Lipo Chemicals, Inc.;

Lipowax PB Pastilles®, which is available from Lipo Chemicals, Inc.;

Mackamine C8®, which is available from The McIntyre Group;

Mackamine C10®, which is available from The Mcintyre Group;

Mackamine C14®, which is available from The Mcintyre Group;

Mackamine CAO®, which is available from The McIntyre Group;

Mackamine CO®, which is available from The McIntyre Group;

Mackamine LO®, which is available from The McIntyre Group;

Mackamine O2®, which is available from The McIntyre Group;

Mackamine SAO®, which is available from The McIntyre Group;

Mackamine SO®, which is available from The Mcintyre Group;

Mazox KCAO®, which is available from BASF Corp.;

Monalac MO®, which is available from Uniqema;

Norfox LDA®, which is available from Norman, Fox & Co.;

Rhodamox LO®, which is available from Rhodia, Inc.;

Schercamox C-AA®, which is available from Noveon®, Inc.;

Schercamox DMA®, which is available from Noveon®, Inc.;

Schercamox DML®, which is available from Noveon®, Inc.;

Schercamox DMM®, which is available from Noveon®, Inc.;

Schercamox DMS®, which is available from Noveon®, Inc.;

Tegotens DO®, which is available from Goldschmidt Chemical Corp.;

Tomah AO-14-2®, which is available from Tomah Products®, Inc.;

Triaminox CDO®, which is available from Tri-Tex Co.®, Inc.;

Mazox CDA CG®; and

Standamox CAW®;

Block Polymers, including but not limited to,

AL 2070®, which is available from Uniqema;

Antarox 17-R-2®, which is available from Rhodia, Inc.;

Antarox 25-R-2®, which is available from Rhodia, Inc.;

Antarox 31-R-1®, which is available from Rhodia, Inc.;

Antarox P-84®, which is available from Rhodia, Inc.;

Antarox P-104/H®, which is available from Rhodia, Inc.;

Arnox BP-Series®, which is available from Crompton Corp.;

Chemonic 435®, which is available from Chemron Corp.;

Chemonic D-25®, which is available from Chemron Corp.;

Chemonic PL Series®, which is available from Chemron Corp.;

Ethox L-121®, which is available from Ethox Chemicals, LLC;

Ethox L-122®, which is available from Ethox Chemicals, LLC;

Genapol PF-10®, which is available from Clariant Corporation;

Genapol PF-20®, which is available from Clariant Corporation;

Genapol PF-40A®, which is available from Clariant Corporation;

Norfox 2-LF®, which is available from Norman, Fox & Co.;

Pluronic, which is available from BASF;

Simulsol NW 342®, which is available from Seppic Inc.;

T-Det BP-1®, which is available from Harcros Chemicals Inc.;

T-Det XD®, which is available from Harcros Chemicals Inc.;

T-Det XH®, which is available from Harcros Chemicals Inc.;

Triton CF-32®, which is available from Dow Chemical Company;

Witconol 171®, which is available from Akzo Nobel Surface Chemistry LLC;

Witconol 324®, which is available from Akzo Nobel Surface Chemistry LLC;

Witconol 324D®, which is available from Akzo Nobel Surface Chemistry LLC;

Witconol PD-2000®, which is available from Akzo Nobel Surface Chemistry LLC;

Chemal BP 261®;

Chemal BP 262®;

Chemal BP 317®;

Chemal BP 3174®;

Pluronic 10R5®;

Pluronic 17R2®;

Pluronic 17R4®;

Pluronic 25R2®;

Pluronic 25R4®;

Pluronic 31R1®;

Pluronic F108®;

Pluronic F127®;

Pluronic F38®;

Pluronic F68®;

Pluronic F68LF®;

Pluronic F77®;

Pluronic F87®;

Pluronic F88®;

Pluronic F98®;

Pluronic L10®;

Pluronic L101®;

Pluronic L121®;

Pluronic L31®;

Pluronic L35®;

Pluronic L43®;

Pluronic L44®;

Pluronic L61®;

Pluronic L62®;

Pluronic L62D®;

Pluronic L62LF®;

Pluronic L64®;

Pluronic L81®;

Pluronic L92®;

Pluronic P103®;

Pluronic P104®;

Pluronic P105®;

Pluronic P123®;

Pluronic P65®;

Pluronic P84®;

Pluronic P85®;

Surfonic POA-17R2®;

Surfonic POA-25R2®;

Tetronic 1107®;

Tetronic 1307®;

Tetronic 150R1®;

Tetronic 304®;

Tetronic 701®;

Tetronic 901®;

Tetronic 904®;

Tetronic 908®; and

Tetronic 90R4®;

Carboxylated Alcohol or Alkylphenol Ethoxylates, including but not limited to,

Emcol CN-6®, which is available from Crompton Corp.;

Ethcarb®, which is available from Ethox Chemicals, LLC;

Gemtex WNT-Conc®, which is available from Finetex Inc.;

Incrodet TD7-C®, which is available from Croda Inc.;

Marlinat CM 105/80®, which is available from Sasol North America Inc.;

Marlowet 1072®, which is available from Sasol North America Inc.;

Marlowet 4530®, which is available from Sasol North America Inc.;

Marlowet 4530 LF®, which is available from Sasol North America Inc.;

Marlowet 4534®, which is available from Sasol North America Inc.;

Marlowet 4538®, which is available from Sasol North America Inc.;

Marlowet 4539®, which is available from Sasol North America Inc.;

Marlowet 4539 LF®, which is available from Sasol North America Inc.;

Marlowet 4541®, which is available from Sasol North America Inc.;

Miranate LEC-80®, which is available from Rhodia, Inc.;

Sandopan B®, which is available from Clariant Corporation;

Sandopan B Modified®, which is available from Clariant Corporation;

Sandopan LS-24 Gel®, which is available from Clariant Corporation;

Surfine T-A®, which is available from Finetex Inc.; and

Surfine AZI-A;

Carboxylic Acids/Fatty Acids, including but not limited to,

Colaterge RAM®, which is available from Colonial Chemical Co.;

Colatrope INC®, which is available from Colonial Chemical Co.;

Crodacid B®, which is available from Croda Inc.;

DeTrope CA-100®, which is available from DeForest Enterprises, Inc.;

Latol MTO®, which is available from Georgia-Pacific Corp.;

Lumulse CC-33 K®, which is available from Lambent Technologies Corp.;

Mulls 2218®, which is available from Bernel Chemical Co.®, Inc.;

OL-600®, which is available from Procter & Gamble;

OL-800®, which is available from Procter & Gamble;

R-910®, which is available from Procter & Gamble;

S-210®, which is available from Procter & Gamble;

Sandopan DTC Acid®, which is available from Clariant Corporation;

Sandopan LS 24 N®, which is available from Clariant Corporation; and

Sandopan MA-18®, which is available from Clariant Corporation;

Ethoxylated Alcohols, including but not limited to,

Adsee 799®, which is available from Akzo Nobel Surface Chemistry LLC;

Adsee 799®, which is available from Crompton Corp.;

Alfonic 610-3.5®, which is available from Sasol North America Inc.;

Alfonic 810-2®, which is available from Sasol North America Inc.;

Alfonic 810-6®, which is available from Sasol North America Inc.;

Alfonic 1012-3®, which is available from Sasol North America Inc.;

Alfonic 1012-5®, which is available from Sasol North America Inc.;

Alfonic 1216CO-1.5®, which is available from Sasol North America Inc.;

Alfonic 1216CO-7®, which is available from Sasol North America Inc.;

Alfonic 1412-3®, which is available from Sasol North America Inc.;

Alfonic 1412-7®, which is available from Sasol North America Inc.;

Arlasolve 200®, which is available from Uniqema;

Arlasolve 200 Liquid®, which is available from Uniqema;

Armix 180-C®, which is available from Crompton Corp.;

Armix 183®, which is available from Crompton Corp.;

Armul 2404®, which is available from Akzo Nobel Surface Chemistry LLC;

Armul 2404®, which is available from Crompton Corp.;

Atlas EMJ-C®, which is available from Atlas Refinery Inc.;

Atlas G-2109®, which is available from Uniqema;

Atlas G-3886®, which is available from Uniqema;

Atlas G-3890®, which is available from Uniqema;

Bio Soft E-200®, which is available from Stepan Canada Inc.;

Bio Soft E-300®, which is available from Stepan Canada Inc.;

Bio Soft E-400®, which is available from Stepan Canada Inc.;

Bio Soft EN 600®, which is available from Stepan Canada Inc.;

Bio Soft TD-400®, which is available from Stepan Canada Inc.;

Bio Soft TD-630®, which is available from Stepan Canada Inc.;

Brij 30®, which is available from Uniqema;

Brij 52®, which is available from Uniqema;

Brij 56®, which is available from Uniqema;

Brij 58®, which is available from Uniqema;

Brij 72®, which is available from Uniqema;

Brij 76®, which is available from Uniqema;

Brij 78®, which is available from Uniqema;

Brij 93®, which is available from Uniqema;

Brij 97®, which is available from Uniqema;

Brij 98®, which is available from Uniqema;

Brij 700®, which is available from Uniqema;

Brij 700 S®, which is available from Uniqema;

Brij 721®, which is available from Uniqema;

Brij 721 S®, which is available from Uniqema;

Burcoterge CDG®, which is available from Burlington Chemical Co.®, Inc.;

Canasol AT 600®, which is available from Canamex Quimicos S.A de C.v;

Canasol AT 800®, which is available from Canamex Quimicos S.A de C.v;

Canasol AT 1200®, which is available from Canamex Quimicos S.A de C.v;

Canasol BJ 35®, which is available from Canamex Quimicos S.A de C.v;

Canasol BJ 36®, which is available from Canamex Quimicos S.A de C.v;

Canasol BJ 52®, which is available from Canamex Quimicos S.A de C.v;

Canasol BJ 58®, which is available from Canamex Quimicos S.A de C.v;

Canasol BJ 72®, which is available from Canamex Quimicos S.A de C.v;

Canasol BJ 78®, which is available from Canamex Quimicos S.A de C.v;

Canasol BJ 98®, which is available from Canamex Quimicos S.A de C.v;

Canasol BJ 307®, which is available-from Canamex Quimicos S.A de C.v;

Cerfak 1400®, which is available from Houghton International Inc.;

Cetomacrogol 1000 BP®, which is available from Croda Inc.;

Chemonic C-2®, which is available from Chemron Corp.;

Chemonic C-10®, which is available from Chemron Corp.;

Chemonic C-20®, which is available from Chemron Corp.;

Chemonic CT-12®, which is available from Chemron Corp.;

Chemonic CT-20®, which is available from Chemron Corp.;

Chemonic CT-30®, which is available from Chemron Corp.;

Chemonic CT-55®, which is available from Chemron Corp.;

Chemonic G-7®, which is available from Chemron Corp.;

Chemonic G-26®, which is available from Chemron Corp.;

Chemonic L-4®, which is available from Chemron Corp.;

Chemonic L-7®, which is available from Chemron Corp.;

Chemonic L-12®, which is available from Chemron Corp.;

Chemonic L-23®, which is available from Chemron Corp.;

Chemonic O-2®, which is available from Chemron Corp.;

Chemonic O-5®, which is available from Chemron Corp.;

Chemonic O-10®, which is available from Chemron Corp.;

Chemonic O-20®, which is available from Chemron Corp.;

Chemonic S-2®, which is available from Chemron Corp.;

Chemonic S-10®, which is available from Chemron Corp.;

Chemonic S-20®, which is available from Chemron Corp.;

Colamulse FE®, which is available from Colonial Chemical Co.;

Cremophor A 20®, which is available from BASF Corp.;

Cremophor SA 2®, which is available from BASF Corp.;

Dehydol 100®, which is available from Cognis Canada Corp.;

Dehydol O-4®, which is available from Cognis Canada Corp.;

Delonic C-18®, which is available from DeForest Enterprises, Inc.;

DeSonic 6T®, which is available from Crompton Corp.;

DeSonic 9D®, which is available from Crompton Corp.;

DeSonic 9T®, which is available from Crompton Corp.;

DeSonic 12D®, which is available from Crompton Corp.;

DeSonic 12T®, which is available from Crompton Corp.;

DeSonic 15T®, which is available from Crompton Corp.;

DeSonic TDA-9®, which is available from Crompton Corp.;

DeThox GLG-7®, which is available from DeForest Enterprises, Inc.;

DeThox GLG-26®, which is available from DeForest Enterprises, Inc.;

DeThox LA-4®, which is available from DeForest Enterprises, Inc.;

DeThox LA-23®, which is available from DeForest Enterprises, Inc.;

DeThox SA-80®, which is available from DeForest Enterprises, Inc.;

Disponil O5®, which is available from Cognis Corporation;

Eccoterge EO-41B®, which is available from Eastern Color & Chemical Co.;

Empilan KA2.5/90®, which is available from Huntsman LLC;

Empilan KA5/90®, which is available from Huntsman LLC;

Empilan KM-20®, which is available from Huntsman LLC;

Empilan KM-50®, which is available from Huntsman LLC;

Empilan L-23®, which is available from Huntsman LLC;

Ethylan 25-3®, which is available from Akzo Nobel Surface Chemistry LLC;

Ethylan 1204®, which is available from Akzo Nobel Surface Chemistry LLC;

Ethylan DA-4®, which is available from Akzo Nobel Surface Chemistry LLC;

Ethylan LA-230®, which is available from Akzo Nobel Surface Chemistry LLC;

Ethylan SN®, which is available from Akzo Nobel Surface Chemistry LLC;

Ethylan TD-60®, which is available from Akzo Nobel Surface Chemistry LLC;

Ethylan TD-100®, which is available from Akzo Nobel Surface Chemistry LLC;

Ethylan TD-1407®, which is available from Akzo Nobel Surface Chemistry LLC;

Eumulgin B1®, which is available from Cognis Canada Corp.;

Eumulgin B2®, which is available from Cognis Canada Corp.;

Eumulgin B3®, which is available from Cognis Canada Corp.;

Eumulgin O-10®, which is available from Cognis Canada Corp.;

Flo Mo 80/20®, which is available from Crompton Corp.;

Flo Mo Low Foam®, which is available from Crompton Corp.;

Forlan C-24®, which is available from RITA Corp.;

Genapol 1454®, which is available from Clariant Corporation;

Genapol BA-020®, which is available from Clariant Corporation;

Genapol BA-040®, which is available from Clariant Corporation;

Genapol C-100®, which is available from Clariant Corporation;

Genapol DA 060®, which is available from Clariant Corporation;

Genapol HS 020®, which is available from Clariant Corporation;

Genapol HS 200®, which is available from Clariant Corporation;

Genapol ID-040®, which is available from Clariant Corporation;

Genapol ID-060®, which is available from Clariant Corporation;

Genapol ID-090®, which is available from Clariant Corporation;

Genapol LA 010®, which is available from Clariant Corporation;

Genapol LA 020®, which is available from Clariant Corporation;

Genapol LA 030®, which is available from Clariant Corporation;

Genapol LA 040®, which is available from Clariant Corporation;

Genapol LA 050®, which is available from Clariant Corporation;

Genapol LA 060®, which is available from Clariant Corporation;

Genapol LA 070®, which is available from Clariant Corporation;

Genapol LA 070S®, which is available from Clariant Corporation;

Genapol LA 230®, which is available from Clariant Corporation;

Genapol O 020®, which is available from Clariant Corporation;

Genapol O 050®, which is available from Clariant Corporation;

Genapol O 100®, which is available from Clariant Corporation;

Genapol O 200®, which is available from Clariant Corporation;

Genapol SA 030®, which is available from Clariant Corporation;

Genapol SA 120®, which is available from Clariant Corporation;

Genapol T-020®, which is available from Clariant Corporation;

Genapol UD-030®, which is available from Clariant Corporation;

Genapol UD-050®, which is available from Clariant Corporation;

Genapol UD-070®, which is available from Clariant Corporation;

Genapol UD-079®, which is available from Clariant Corporation;

Genapol UD-080®, which is available from Clariant Corporation;

Genapol UD-110®, which is available from Clariant Corporation;

Genapol X 030®, which is available from Clariant Corporation;

Genapol X 050®, which is available from Clariant Corporation;

Genapol X 060®, which is available from Clariant Corporation;

Genapol X 070®, which is available from Clariant Corporation;

Genapol X 080®, which is available from Clariant Corporation;

Genapol X 100®, which is available from Clariant Corporation;

Genapol X159®, which is available from Clariant Corporation;

Generol 122 E5®, which is available from Cognis Canada Corp.;

Generol 122 E25®, which is available from Cognis Canada Corp.;

Hostacerin T-3®, which is available from Clariant Corporation;

Iconol LF 110®, which is available from BASF Corp.;

Incropol CS-20®, which is available from Croda Inc.;

Lexemul CS-20®, which is available from Inolex Chemical Co.;

Liponic EG-1®, which is available from Lipo Chemicals, Inc.;

Lipowax D®, which is available from Lipo Chemicals, Inc.;

Lipowax G®, which is available from Lipo Chemicals, Inc.;

Lipowax NI®, which is available from Lipo Chemicals, Inc.;

Lipowax P®, which is available from Lipo Chemicals, Inc.;

Lipowax P-31®, which is available from Lipo Chemicals, Inc.;

Lipowax PR®, which is available from Lipo Chemicals, Inc.;

Lumulse CS-20, which is available from Lambent Technologies Corp.;

Macol CSA-20®, which is available from BASF Corp.;

Marlox B 24/50®, which is available from Sasol North America Inc.;

Mazawet 77®, which is available from BASF Corp.;

Norfox 1713®, which is available from Norman, Fox & Co.;

Norfox 2579®, which is available from Norman, Fox & Co.;

Norfox Lo Foam®, which is available from Norman, Fox & Co.;

Promulgen D®, which is available from Amerchol Corp.;

Promulgen G®, which is available from Amerchol Corp.;

Renex 30®, which is available from Uniqema;

Renex 36®, which is available from Uniqema;

Rhodasurf A 24®, which is available from Rhodia, Inc.;

Rhodasurf ME-10®, which is available from Rhodia, Inc.;

Rhodasurf BEH-25®, which is available from Rhodia, Inc.;

Rhodasurf BEH-40®, which is available from Rhodia, Inc.;

Rhodasurf DA 530®, which is available from Rhodia, Inc.;

Rhodasurf DA 630®, which is available from Rhodia, Inc.;

Rhodasurf DA 639®, which is available from Rhodia, Inc.;

Rhodasurf LAN-23®, which is available from Rhodia, Inc.;

Rhodasurf ON-870®, which is available from Rhodia, Inc.;

Rhodasurf ON-877®, which is available from Rhodia, Inc.;

Rhodasurf TB-970 FLK®, which is available from Rhodia, Inc.;

Ritacet-20®, which is available from RITA Corp.;

Ritachol 1000®, which is available from RITA Corp.;

Ritachol 2000®, which is available from RITA Corp.;

Ritachol 5000®, which is available from RITA Corp.;

Ritox 35®, which is available from RITA Corp.;

Surfonic DA-4®, which is available from Huntsman LLC;

Surfonic DA-6®, which is available from Huntsman LLC;

Surfonic L46-7®, which is available from Huntsman LLC;

Surfonic POA®, which is available from Huntsman LLC;

Synthrapol KB®, which is available from Uniqema;

Teginacid®, which is available from Goldschmidt Chemical Corp.;

Teginacid C®, which is available from Goldschmidt Chemical Corp.;

Tegotens EC 11®, which is available from Goldschmidt Chemical Corp.;

Tinegal NA®, which is available from Ciba Specialty Chemicals Corp.;

Tomadol 400®, which is available from Tomah Products®, Inc.;

Tomadol 600®, which is available from Tomah Products®, Inc.;

Tomadol 900®, which is available from Tomah Products®, Inc.;

Uniperol O®, which is available from BASF Corp.;

Witconol SN Series®, which is available from Crompton Corp.;

T-Det EPO-64®;

T-Det N-1.5®;

T-Det N-10.5®;

T-Det N-100®;

T-Det N-1007®;

T-Det N-12®;

T-Det N-14®;

T-Det N-20®;

T-Det N-30®;

T-Det N-307®;

T-Det N-4®;

T-Det N-40®;

T-Det N-407®;

T-Det N-50®;

T-Det N-507®;

T-Det N-6®;

T-Det N-70®;

T-Det N-8®;

T-Det N-9.5®;

T-Det O-12®;

Abex 2515®;

Abex 2525/40®;

Abex 2535®;

Abex 2545®;

Alfonic 1216CO-9®;

Alfonic 1412-3®;

Alfonic TDA-12®;

Alfonic TDA-3®;

Alfonic TDA-4®;

Alfonic TDA-6®;

Alfonic TDA-7®;

Alfonic TDA-8®;

Alfonic TDA-9®;

Ameroxol OE-10®;

Ameroxol OE-2®;

Ameroxol OE-20®;

Armix 176®;

Armix 180-C®;

Armix 183®;

Brij 35®;

Brij 35 Liquid®;

Brij 35 SP®;

Canasol MJ 2109®;

Canasol MJ 52®;

Canasol R 3600®;

Canasol R 3603®;

Canasol R 4000 H®;

Chemal DA-4®;

Chemal DA-6®;

Chemal DA-9®;

Chemal LA-23®;

Chemal LA-4®;

Chemal LA-9®;

Chemal OA-20G®;

Chemal OA-5®;

Chemal OA-9®;

Chemal TDA-12®;

Chemal TDA-15®;

Chemal TDA-3®;

Chemal TDA-6®;

Chemal TDA-9®;

DeSonic TDA-9®;

Ethal EH-2®;

Ethal EH-5®;

Ethal OA-23®;

Ethosperse CA-20®;

Ethosperse LA-12®;

Ethosperse LA-23®;

Ethosperse LA-4®;

Ethylan SN-120®;

Ethylan SN-70®;

Ethylan SN-90®;

Generol 122 E25®;

Hetoxol C-24®;

Hetoxol CA-10®;

Hetoxol CA-2®;

Hetoxol CA-20®;

Hetoxol CAWS®;

Hetoxol CD-3®;

Hetoxol CD-4®;

Hetoxol CS-15®;

Hetoxol CS-20®;

Hetoxol CS-20D®;

Hetoxol CS-25®;

Hetoxol CS-50®;

Hetoxol CS-9®;

Hetoxol CSA-15®;

Hetoxol D®;

Hetoxol G®;

Hetoxol J®;

Hetoxol L®;

Hetoxol L-23®;

Hetoxol L-4®;

Hetoxol L-9®;

Hetoxol LS-9®;

Hetoxol M-3®;

Hetoxol OA-10 Special®;

Hetoxol OA-20 Special®;

Hetoxol OA-3 Special®;

Hetoxol OA-5 Special®;

Hetoxol OL-10®;

Hetoxol OL-2®;

Hetoxol OL-23®;

Hetoxol OL-4®;

Hetoxol OL-40®;

Hetoxol OL-5®;

Hetoxol PLA®;

Hetoxol SP-15®;

Hetoxol STA-10®;

Hetoxol STA-2®;

Hetoxol STA-30®;

Hetoxol TD-12®;

Hetoxol TD-18®;

Hetoxol TD-3®;

Hetoxol TD-6®;

Hetoxol TD-9®;

Hetoxol TDEP-15®;

Hetoxol TDEP-63®;

Iconol 24-12®;

Iconol 24-23®;

Iconol 24-4®;

Iconol DA-4®;

Iconol DA-6®;

Iconol TDA-10®;

Iconol TDA-3®;

Iconol TDA-6®;

Iconol TDA-8-90%®;

Iconol TDA-9®;

Lexemul CS-20®;

Lipocol C-10®;

Lipocol C-2®;

Lipocol C-20®;

Lipocol L-12®;

Lipocol L-23®;

Lipocol L-4®;

Lipocol O-10®;

Lipocol O-2®;

Lipocol O-20®;

Lipocol O-5®;

Lipocol S-10®;

Lipocol S-2®;

Lipocol S-20®;

Lipocol SC-15®;

Lipocol SC-20®;

Lipocol SC-4®;

Lipocol TD-12®;

Marlox B 24/80®;

Myrj 45®;

Myrj 52®;

Myrj 52S®;

Myrj 53®;

Myrj 59®;

Plurafac A-38®;

Plurafac B-25-5®;

Plurafac B-26®;

Plurafac D-25®;

Plurafac LF 131®;

Plurafac LF 4030®;

Plurafac LF 7000®;

Plurafac RA-20®;

Plurafac RA-30®;

Plurafac RA-40®;

Rhodasurf BC-420®;

Rhodasurf BC-610®;

Rhodasurf BC-630®;

Rhodasurf BC-720®;

Rhodasurf BC-840®;

Rhodasurf L 1®;

Rhodasurf L 12®;

Rhodasurf L 2®;

Rhodasurf L 20®;

Rhodasurf L 3®;

Rhodasurf L 30®;

Rhodasurf L 4®;

Rhodasurf L 50®;

Rhodasurf L 7/90®;

Rhodasurf LA-3®;

Rhodasurf LA-7®;

Rhodasurf LAN-23®;

Rhodasurf ON-870®;

Rhodasurf ON-877®;

Rhodasurf TDA-8/5®;

Ritapro 100®;

Ritapro 165®;

Ritapro 200®;

Ritapro 300®;

Ritoleth 10®;

Ritoleth 2®;

Ritoleth 20®;

Ritoleth 5®;

Solulan 16®;

Solulan 75®;

Solulan C-24®;

Solulan L-575®;

Surfonic TDA-11®;

Surfonic TDA-3B®;

Surfonic TDA-6®;

Surfonic TDA-6/88®;

Surfonic TDA-8®;

Surfonic TDA-8/90®;

Surfonic TDA-9®;

T-Det A106®;

T-Det A109®;

T-Det A136®;

T-Det A139®;

T-Det A2412®;

T-Det A243®;

T-Det A247®;

T-Det A249®;

T-Det A467®;

T-Det COE®;

T-Det DD-10®;

T-Det DD-5®;

T-Det DD-7®;

T-Det EPO-104®;

T-Det EPO-61®;

T-Det EPO-62®;

T-Det O-165®;

T-Det O-307®;

T-Det O-40®;

T-Det O-407®;

T-Dei O-6®;

T-Det O-8®;

T-Det O-9®;

Tergitol 15-S-12®;

Tergitol 15-S-15®;

Tergitol 15-S-20®;

Tergitol 15-S-20 (80AQ)®;

Tergitol 15-S-3®;

Tergitol 15-S-30®;

Tergitol 15-S-40®;

Tergitol 15-S-40 (70AQ)®;

Tergitol 15-S-5®;

Tergitol 15-S-7®;

Tergitol 15-S-9®;

Tergitol L-101®;

Tergitol L-61®;

Tergitol L-62®;

Tergitol L-64®;

Tergitol L-81®;

Tergitol NP-10®;

Tergitol NP-11®;

Tergitol NP-12®;

Tergitol NP-13®;

Tergitol NP-15®;

Tergitol NP-30®;

Tergitol NP-30 (70 AQ)®;

Tergitol NP-4®;

Tergitol NP-40®;

Tergitol NP-40 (70 AQ)®;

Tergitol NP-50 (70 AQ)®;

Tergitol NP-55 (70 AQ)®;

Tergitol NP-6®;

Tergitol NP-7®;

Tergitol NP-70 (70 AQ)®;

Tergitol NP-8®;

Tergitol NP-9®;

Tergitol NP-9.5®;

Tergitol TMN-10®;

Tergitol TMN-3®;

Tergitol TMN-6®;

Tergitol XD®;

Tergitol XH®;

Tergitol XJ®;

Tomadol 1-3®;

Tomadol 1-5®;

Tomadol 1-7®;

Tomadol 23-1®;

Tomadol 23-3®;

Tomadol 23-5®;

Tomadol 23-6.5®;

Tomadol 25-12®;

Tomadol 25-3®;

Tomadol 25-7®;

Tomadol 25-9®;

Tomadol 45-13®;

Tomadol 45-2.5®;

Tomadol 45-7®;

Tomadol 91-2.5®;

Tomadol 91-6®;

Tomadol 91-8®;

Volpo S-10®;

Volpo S-2®;

Volpo S-20®;

Volpo-10®;

Volpo-20®;

Volpo-3®; and

Volpo-5®;

Ethoxylated Alkylphenols, including but not limited to,

Antarox LF-222®, which is available from Rhodia, Inc.;

Atlox 775®, which is available from Uniqema;

Caloxylate N-9®, which is available from Pilot Chemical Co.;

Canasol NF-1000®, which is available from Canamex Quimicos S.A de C.v;

Canasol NF-3000®, which is available from Canamex Quimicos S.A de C.v;

Canasol NF-3070®, which is available from Canamex Quimicos S.A de C.v;

Canasol OF 1670®, which is available from Canamex Quimicos S.A de C.v;

Canasol OF 2570®, which is available from Canamex Quimicos S.A de C.v;

Canasol OF 4070®, which is available from Canamex Quimicos S.A de C.v;

Chemax DNP-8®, which is available from Chemax Performance Solutions;

Chemax DNP-18®, which is available from Chemax Performance Solutions;

Chemax DNP-150/50®, which is available from Chemax Performance Solutions;

DeSonic 1.5N®, which is available from Crompton Corp.;

DeSonic 4N®, which is available from Crompton Corp.;

DeSonic 5N®, which is available from Crompton Corp.;

DeSonic 6D®, which is available from Crompton Corp.;

DeSonic 6N®, which is available from Crompton Corp.;

DeSonic 7N®, which is available from Crompton Corp.;

DeSonic 9N®, which is available from Crompton Corp.;

DeSonic 10D®, which is available from Crompton Corp.;

DeSonic 11N®, which is available from Crompton Corp.;

DeSonic 12N®, which is available from Crompton Corp.;

DeSonic 15N®, which is available from Crompton Corp.;

DeSonic 20N®, which is available from Crompton Corp.;

Eccoscour RC®, which is available from Eastern Color & Chemical Co.;

Eccoterge EO-100®, which is available from Eastern Color & Chemical Co.;

Emulsifier 632/90%®, which is available from Ethox Chemicals, LLC;

Geronol AG-821®, which is available from Rhodia, Inc.;

Gradonic N-95®, which is available from Graden Chemical Co. Inc.;

Hetoxide NP-4®, which is available from Global-Seven, Inc.;

Hetoxide NP-30®, which is available from Global-Seven, Inc.;

Hostapal N-100®, which is available from Clariant Corporation;

Hostapal N-110®, which is available from Clariant Corporation;

Igepal CTA-639W®, which is available from Rhodia, Inc.;

Igepal DAP-9®, which is available from Rhodia, Inc.;

Igepal OD-410®, which is available from Rhodia, Inc.;

Igepal SS-837®, which is available from Rhodia, Inc.;

Lipocol NP-9 USP®, which is available from Lipo Chemicals, Inc.;

Macol DNP-10®, which is available from BASF Corp.;

Marlophen NP 5®, which is available from Sasol North America Inc.;

Marlophen P 1®, which is available from Sasol North America Inc.;

Surfonic NB®, which is available from Huntsman LLC;

Surfonic OPB-307®, which is available from Huntsman LLC;

Surfonic OPB-407®, which is available from Huntsman LLC;

Syn Fac 334®, which is available from Milliken Chemical;

Syn Fac 8216®, which is available from Milliken Chemical;

Triton N-57®, which is available from Dow Chemical Company;

Trycol 6956®, which is available from Cognis Corporation;

Trycol 6961®, which is available from Cognis Corporation;

Trycol 6964®, which is available from Cognis Corporation;

Trycol 6969®, which is available from Cognis Corporation;

Trycol 6974®, which is available from Cognis Corporation;

Witbreak DRB-127®, which is available from Akzo Nobel Surface Chemistry LLC;

Witbreak DRB-127®, which is available from Crompton Corp.; and

Witconol NP Series®, which is available from Akzo Nobel Surface Chemistry LLC;

Delonic NPE-100®;

Delonic NPE-30®;

Delonic NPE-40®;

Delonic OPE-10®;

Delonic OPE-12®;

Delonic OPE-30®;

Delonic OPE-40®;

Delonic OPE-5®;

Delonic OPE-7.5®;

DeSonic S-100®;

DeSonic S-114®;

DeSonic S-405®;

DeSonic S-45®;

Igepal CA-210®;

Igepal CA-407®;

Igepal CA-420®;

Igepal CA-520®;

Igepal CA-620®;

Igepal CA-630®;

Igepal CA-720®;

Igepal CA-877®;

Igepal CA-887®;

Igepal CA-897®;

Igepal CO-210®;

Igepal CO-430®;

Igepal CO-520®;

Igepal CO-530®;

Igepal CO-610®;

Igepal CO-630®;

Igepal CO-630 Special®;

Igepal CO-660®;

Igepal CO-710®;

Igepal CO-720®;

Igepal CO-730®;

Igepal CO-738®;

Igepal CO-850®;

Igepal CO-880®;

Igepal CO-887®;

Igepal CO-890®;

Igepal CO-897®;

Igepal CO-970®;

Igepal CO-977®;

Igepal CO-987®;

Igepal CO-990 FLK®;

Igepal CO-997®;

Igepal DM-430®;

Igepal DM-530®;

Igepal DM-710®;

Igepal DM-730®;

Igepal DM-880®;

Igepal DM-970 FLK®;

Igepal OD-410®;

Igepal RC-520®;

Igepal RC-620®;

Igepal RC-630®;

Norfox NP-12®;

Norfox NP-4®;

Norfox NP-6®;

Norfox NP-887®;

Norfox NP-9®;

Norfox NP-977®;

Norfox OP-100®;

Norfox OP-102®;

Norfox OP-114®;

Norfox OP-45®;

Surfonic N-10®;

Surfonic N-100®;

Surfonic N-102®;

Surfonic N-120®;

Surfonic N-150®;

Surfonic N-200®;

Surfonic N-300®;

Surfonic N-31.5®;

Surfonic N-40®;

Surfonic N-400®;

Surfonic N-60®;

Surfonic N-85®;

Surfonic N-95®;

Surfonic NB-18®;

Surfonic NB-23®;

Surfonic NB-25®;

Surfonic NB-307®;

Surfonic NB-31®;

Surfonic NB-41®;

Surfonic NB-43®;

Surfonic OP-100®;

Surfonic OP-120®;

Surfonic OP-15®;

Surfonic OP-35®;

Surfonic OP-70®;

Triton X-100®;

Triton X-102®;

Triton X-114®;

Triton X-15®;

Triton X-165 (70%)®;

Triton X-207®;

Triton X-305 (70%)®;

Triton X-35®;

Triton X-405 (70%)®;

Triton X-45®; and

Triton X-705 (70%)®;

Ethoxylated Aryl Phenols, including but not limited to,

Soprophor BSU®, which is available from Rhodia, Inc.;

Soprophor CY/8®, which is available from Rhodia, Inc.;

Soprophor S/25®, which is available from Rhodia, Inc.;

Witconol NIO®, which is available from Akzo Nobel Surface Chemistry LLC;

Witconol NIW®, which is available from Akzo Nobel Surface Chemistry LLC;

Witconol S-100®, which is available from Akzo Nobel Surface Chemistry LLC;

Cedepal CA-210®;

Cedepal CA-520®;

Cedepal CA-630®;

Cedepal CA-720®;

Cedepal CA-890®;

Cedepal CA-897®;

Cedepal CO-210®;

Cedepal CO-430®;

Cedepal CO-530®;

Cedepal CO-630®;

Cedepal CO-710®;

Cedepal CO-730®;

Cedepal CO-880®;

Cedepal CO-887®;

Cedepal CO-890®;

Cedepal CO-897®;

Cedepal CO-970®;

Cedepal CO-977®;

Cedepal CO-990®; and

Cedepal CO-997®;

Ethoxylated Fatty Acids, including but not limited to,

Aldo PGHMS®, which is available from Lonza Inc.;

Alkamuls TO-15/HR®, which is available from Rhodia, Inc.;

Armotan AL-69-66®, which is available from Akzo Nobel Surface Chemistry LLC;

Cerasynt 840®, which is available from International Specialty Products/IS;

Cerasynt 945®, which is available from International Specialty Products/IS;

Crystal Inhibitor No. 5®, which is available from Harcros Chemicals Inc.;

DeThox Acid L-9®, which is available from DeForest Enterprises, Inc.;

DeThox Acid S-8®, which is available from DeForest Enterprises, Inc.;

Ethofat 242/25®, which is available from Akzo Nobel Surface Chemistry LLC;

Hydropalat 65®, which is available from Cognis Corporation;

Lipo EGMS®, which is available from Lipo Chemicals, Inc.;

Lipopeg 2 DL®, which is available from Lipo Chemicals, Inc.;

Lipopeg 4 DL®, which is available from Lipo Chemicals, Inc.;

Lipopeg 4-L®, which is available from Lipo Chemicals, Inc.;

Lipopeg 39-S®, which is available from Lipo Chemicals, Inc.;

Lipopeg 4-S®, which is available from Lipo Chemicals, Inc.;

Lipopeg 10-S®, which is available from Lipo Chemicals, Inc.;

Lipopeg 100-S®, which is available from Lipo Chemicals, Inc.;

Lipopeg 6000 DS®, which is available from Lipo Chemicals, Inc.;

Lumulse 40-L®, which is available from Lambent Technologies Corp.;

Lumulse 40-S®, which is available from Lambent Technologies Corp.;

Lumulse 42-L®, which is available from Lambent Technologies Corp.;

Lumulse 42-S®, which is available from Lambent Technologies Corp.;

Lumulse 100-S®, which is available from Lambent Technologies Corp.;

Lumulse 602-S®, which is available from Lambent Technologies Corp.;

Magrabar PGE-20-0®, which is available from Magrabar Chemical Corp.;

Magrabar PGE-20L®, which is available from Magrabar Chemical Corp.;

Magrabar PGE-20T®, which is available from Magrabar Chemical Corp.;

Magrabar PGE-22-0®, which is available from Magrabar Chemical Corp.;

Magrabar PGE-22L®, which is available from Magrabar Chemical Corp.;

Magrabar PGE-22T®, which is available from Magrabar Chemical Corp.;

Magrabar PGE-40-0®, which is available from Magrabar Chemical Corp.;

Magrabar PGE-40L®, which is available from Magrabar Chemical Corp.;

Magrabar PGE-40T®, which is available from Magrabar Chemical Corp.;

Magrabar PGE-42-0®, which is available from Magrabar Chemical Corp.;

Magrabar PGE-42L®, which is available from Magrabar Chemical Corp.;

Magrabar PGE-42T®, which is available from Magrabar Chemical Corp.;

Magrabar PGE-60-0®, which is available from Magrabar Chemical Corp.;

Magrabar PGE-60L®, which is available from Magrabar Chemical Corp.;

Magrabar PGE-60T®, which is available from Magrabar Chemical Corp.;

Magrabar PGE-62-0®, which is available from Magrabar Chemical Corp.;

Magrabar PGE-62L®, which is available from Magrabar Chemical Corp.;

Magrabar PGE-62T®, which is available from Magrabar Chemical Corp.;

Mapeg S-40K®, which is available from BASF Corp.;

Marlowet OTS®, which is available from Sasol North America Inc.;

Naturechem PGR®, which is available from CasChem®, Inc.;

PG No. 4®, which is available from Hart Chemical Corp.;

Renex 20®, which is available from Uniqema;

Ritox 52®, which is available from RITA Corp.;

Ritox 53®, which is available from RITA Corp.;

Ritox 59®, which is available from RITA Corp.;

Surfax 8916/A®, which is available from Houghton International Inc.;

Tego Acid S 40 P®, which is available from Goldschmidt Chemical Corp.;

Tego Acid S 100 P®, which is available from Goldschmidt Chemical Corp.;

Tween 20®, which is available from Uniqema;

Volpo 131®, which is available from Croda Inc.;

Alkamuls 400-MO/E®;

DeThox Acid O-14®;

DeThox Acid O-16®;

DeThox Acid O-9®;

DeThox Acid TO-14®;

DeThox Acid TO-16.5®;

DeThox Acid TO-8.5®;

Emerest 2704®;

Emerest 2712®;

Emerest 2715®;

Ethox MA-8®;

Ethox MI-14®;

Ethox ML-14®;

Ethox ML-5®;

Ethox MO-14®;

Ethox MO-9®;

Ethox MS-14®;

Ethox MS-23®;

Ethox MS-40®;

Ethox MS-8®;

Ethox TO-16®;

Ethox TO-8®;

Flexricin 13®;

Flexricin 15®;

Genapol 1261®;

Lumulse 40-O K®;

Lumulse 42-O K®;

Lumulse GMT-40®;

Lumulse PEG 1450®;

Lumulse PEG 1450 NF®;

Lumulse PEG 8000®;

Lumulse PGO®;

Lumulse POE (2) Oleyl Amine®;

Magrabar PGO-104®;

Mapeg 200 ML®;

Mapeg 400 MO®;

Mapeg 400 MOT®;

Mapeg 400DO®;

Mapeg 400DOT®;

Mapeg 600DOT®;

Marlox FK 86®;

Marlox MO 154®;

Surfonic L12-3®;

Surfonic L12-6®;

Surfonic L12-8®;

Surfonic L24-12®;

Surfonic L24-2®;

Surfonic L24-22®;

Surfonic L24-3®;

Surfonic L24-4®;

Surfonic L24-5®;

Surfonic L24-7®;

Surfonic L24-7.1®;

Surfonic L24-9®;

Surfonic LF-17®;

Surfonic LF-18®;

Surfonic LF-37®;

Surfonic LF-41®;

Surfonic LF-42®;

Surfonic LF-47®;

Surfonic LF-68®;

Surfonic P1®;

Surfonic P3®;

Surfonic P5®; and

Surfonic P6®;

Ethoxylated Fatty Esters or Oils (Animal & Veg.), including but not limited to,

Acconon 6-C10®, which is available from Abitec Corporation;

Acconon CC-6®, which is available from Abitec Corporation;

Acconon CO-7®, which is available from Abitec Corporation;

Aldosperse 40/60 FG®, which is available from Lonza Inc.;

Aldosperse ML-23®, which is available from Lonza Inc.;

Aldosperse MS-20 FG®, which is available from Lonza Inc.;

Alkamuls EL-620®, which is available from Rhodia, Inc.;

Alkamuls EL-719®, which is available from Rhodia, Inc.;

Alkamuls EL-985®, which is available from Rhodia, Inc.;

Arlatone G®, which is available from Uniqema;

Arlatone T®, which is available from Uniqema;

Atlas G-1045A®, which is available from Uniqema;

Atlas G-1086®, which is available from Uniqema;

Atlas G-1087®, which is available from Uniqema;

Atlas G-1089®, which is available from Uniqema;

Atlas G-1096®, which is available from Uniqema;

Atlas G-1292®, which is available from Uniqema;

Atlas G-1293®, which is available from Uniqema;

Atlas G-1300®, which is available from Uniqema;

Atlas G-7076®, which is available from Uniqema;

Capmul EMG®, which is available from Abitec Corporation;

Chemonic CO-40®, which is available from Chemron Corp.;

Chemonic LI-3®, which is available from Chemron Corp.;

Chemonic LI-7®, which is available from Chemron Corp.;

Cirrasol GM®, which is available from Uniqema;

Cremophor CO 40®, which is available from BASF Corp.;

Cremophor CO 410®, which is available from BASF Corp.;

Cremophor EL®, which is available from BASF Corp.;

Cremophor GC7®, which is available from BASF Corp.;

Cremophor RH-40®, which is available from BASF Corp.;

Crovol A-40®, which is available from Croda Inc.;

Crovol A-70®, which is available from Croda Inc.;

Crovol M-70®, which is available from Croda Inc.;

Crovol PK-70®, which is available from Croda Inc.;

Cutina E-24®, which is available from Cognis Canada Corp.;

Dacospin 12-R®, which is available from Cognis Corporation;

Dehymuls HRE-7®, which is available from Cognis Corporation;

DeSonic 30C®, which is available from Crompton Corp.;

DeSonic 36C®, which is available from Crompton Corp.;

DeSonic 40C®, which is available from Crompton Corp.;

Durfax 60®, which is available from Loders Croklaan U.S.A.;

Durfax 65®, which is available from Loders Croklaan U.S.A.;

Durfax 80®, which is available from Loders Croklaan U.S.A.;

Durfax EOM®, which is available from Loders Croklaan U.S.A.;

Eccoterge NF-2®, which is available from Eastern Color & Chemical Co.;

Emulpon CO-360®, which is available from Akzo Nobel Surface Chemistry LLC;

Emulpon CO-550®, which is available from Akzo Nobel Surface Chemistry LLC;

Emulsogen EL®, which is available from Clariant Corporation;

Emulsogen HCO 040®, which is available from Clariant Corporation;

Emulsogen HCO 060®, which is available from Clariant Corporation;

Emulsynt 1055®, which is available from International Specialty Products/IS;

Ethox 3095®, which is available from Ethox Chemicals, LLC;

Eumulgin RO-40®, which is available from Cognis Canada Corp.;

Genapol G-260®, which is available from Clariant Corporation;

Glycosperse L-20®, which is available from Lonza Inc.;

Glycosperse O-5®, which is available from Lonza Inc.;

Glycosperse O-20®, which is available from Lonza Inc.;

Glycosperse O-20 FG®, which is available from Lonza Inc.;

Glycosperse S-20®, which is available from Lonza Inc.;

Glycosperse S-20 FG®, which is available from Lonza Inc.;

Glycosperse TS-20®, which is available from Lonza Inc.;

Glycosperse TS-20 FG®, which is available from Lonza Inc.;

Hetan SL®, which is available from Global-Seven, Inc.;

Hetan SO®, which is available from Global-Seven, Inc.;

Hetan SS®, which is available from Global-Seven, Inc.;

Hetoxide C-2®, which is available from Global-Seven, Inc.;

Hetoxide C-9®, which is available from Global-Seven, Inc.;

Hetoxide C-15®, which is available from Global-Seven, Inc.;

Hetoxide C-25®, which is available from Global-Seven, Inc.;

Hetoxide C-40®, which is available from Global-Seven, Inc.;

Hetoxide C-200®, which is available from Global-Seven, Inc.;

Hetoxide C-200-50%®, which is available from Global-Seven, Inc.;

Hetoxide GC-30®, which is available from Global-Seven, Inc.;

Hetoxide HC-60®, which is available from Global-Seven, Inc.;

Ice No. 2®, which is available from Loders Croklaan U.S.A.;

Incrocas 30/40®, which is available from Croda Inc.;

Lexol EC®, which is available from Inolex Chemical Co.;

Lexol EO®, which is available from Inolex Chemical Co.;

Lipocol HCO-40®, which is available from Lipo Chemicals, Inc.;

Lipocol HCO-60®, which is available from Lipo Chemicals, Inc.;

Lipocol O-3 Special®, which is available from Lipo Chemicals, Inc.;

Lipopeg 2-L®, which is available from Lipo Chemicals, Inc.;

Lipopeg 4-DO®, which is available from Lipo Chemicals, Inc.;

Lipopeg 4-DS®, which is available from Lipo Chemicals, Inc.;

Lipovol GTB®, which is available from Lipo Chemicals, Inc.;

Lonzest SML-20®, which is available from Lonza Inc.;

Lonzest SMO-20®, which is available from Lonza Inc.;

Lonzest SMS-20®, which is available from Lonza Inc.;

Lonzest STO-20®, which is available from Lonza Inc.;

Lonzest STS-20®, which is available from Lonza Inc.;

Lumulse GR-40®, which is available from Lambent Technologies Corp.;

Lumulse GRH-40®, which is available from Lambent Technologies Corp.;

Lumulse POE (7) GML®, which is available from Lambent Technologies Corp.;

Lumulse POE (12) Glyc®, which is available from Lambent Technologies Corp.;

Lumulse POE (40) MS KP®, which is available from Lambent Technologies Corp.;

Marlowet 4750®, which is available from Sasol North America Inc.;

Marlowet LVS®, which is available from Sasol North America Inc.;

Marlowet R 11®, which is available from Sasol North America Inc.;

Marlowet R 40®, which is available from Sasol North America Inc.;

Mazol 80 MGK®, which is available from BASF Corp.;

Nonionic Emulsifier T-9®, which is available from Werner G. Smith Inc.;

Oronal LCG®, which is available from Seppic Inc.;

Polyderm PPI-CO-200®, which is available from Alzo International, Inc.;

Polyderm PPI-CO-40®, which is available from Alzo International, Inc.;

Rewoderm LI 520-70®, which is available from Goldschmidt Chemical Corp.;

Ritapeg 150 DS®, which is available from RITA Corp.;

Softigen 767®, which is available from Sasol North America Inc.;

Surfactol 318®, which is available from CasChem®, Inc.;

Surfactol 365®, which is available from CasChem®, Inc.;

Syn Lube 107®, which is available from Milliken Chemical;

Syn Lube 728®, which is available from Milliken Chemical;

Syn Lube 1632H®, which is available from Milliken Chemical;

Syn Lube 6277-A®, which is available from Milliken Chemical;

T-Det C-20®, which is available from Harcros Chemicals Inc.;

T-Det C-40®, which is available from Harcros Chemicals Inc.;

Tally 100 Plus®, which is available from Loders Croklaan U.S.A.;

Uniperol EL®, which is available from BASF Corp.;

Acconon E®;

Chemax CO-16®;

Chemax CO-200/50®;

Chemax CO-25®;

Chemax CO-30®;

Chemax CO-36®;

Chemax CO-40®;

Chemax CO-5®;

Chemax CO-80®;

Chemax DN P-150/50®;

Chemax DNP-18®;

Chemax DNP-8®;

Chemax E 200 ML®;

Chemax E 200 MO®;

Chemax E 400 ML®;

Chemax E 400 MO®;

Chemax E 600 ML®;

Chemax E 600 MO®;

Chemax E 1000 MO®;

Chemax E 1000 MS®;

Chemax E200 MS®;

Chemax E400 MS®;

Chemax E600 MS®;

Chemax HCO-16®;

Chemax HCO-200/50®;

Chemax HCO-25®;

Chemax HCO-5®;

Chemax TO-16®;

Chemax TO-8®;

Colalube 3409®;

Colalube 3411®;

Colalube 3413®;

Colalube 3414®;

Colalube 3415®;

Colalube 3416®;

Colalube 3417®;

DePeg 16-CO®;

DePeg 25-CO®;

DePeg 30-CO®;

DePeg 40-CO®;

DePeg 5-CO®;

DePeg 80-CO®;

Ethox 3095®;

Ethox CO-16®;

Ethox CO-200®;

Ethox CO-25®;

Ethox CO-30®;

Ethox CO-36®;

Ethox CO-40®;

Ethox CO-5®;

Hetoxide BN-13®;

Hetoxide DNP-4®;

Hetoxide DNP-9.6®;

Hetoxide G-26®;

Hetoxide G-7®;

Hetoxide GT-20®;

Hetoxide HC-40®;

Lipo GMS 450®;

Lipo Polyglycol 1000®;

Lipo Polyglycol 200®;

Lipo Polyglycol 300®;

Lipo Polyglycol 3350®;

Lipo Polyglycol 400®;

Lipo Polyglycol 600®;

Surfonic CO-15®;

Surfonic CO-25®;

Surfonic CO-30®;

Surfonic CO-36®;

Surfonic CO-42®;

Surfonic TX-CCN®;

Tagat CH 40®;

Tagat CH 60®;

Tagat L 2®;

Tagat S®;

Tagat S 2®; and

Tally 100 Plus®;

Fatty Esters, including but not limited to,

Actralube-Syn 147®, which is available from Georgia-Pacific Corp.;

Atlas G-1556®, which is available from Uniqema;

Atlas G-1564®, which is available from Uniqema;

Atlasol Base Oil S®, which is available from Atlas Refinery Inc.;

Base ML®, which is available from Keil Chemical;

Base MT®, which is available from Keil Chemical;

Cerasynt 303®, which is available from International Specialty Products/IS;

Dermol 1012®, which is available from Alzo International, Inc.;

Kemester 4000®, which is available from Crompton Corp.;

Lactipol S®, which is available from Canamex Quimicos S.A de C.v;

Magrabar PGO®, which is available from Magrabar Chemical Corp.;

Mayco Base BFO®, which is available from Dover Chemical Corp.;

Methyl Linoleate®, which is available from Hart Chemical Corp.;

Pationic 122A®, which is available from RITA Corp.;

Pationic 138C®, which is available from RITA Corp.;

Pationic CSL®, which is available from RITA Corp.;

Pationic ISL®, which is available from RITA Corp.;

Pationic SBL®, which is available from RITA Corp.;

Pationic SSL®, which is available from RITA Corp.;

Ritasol®, which is available from RITA Corp.;

Tego Alkanol CS 20®, which is available from Goldschmidt Chemical Corp.;

Tego Alkanol L23 P®, which is available from Goldschmidt Chemical Corp.;

Tego Alkanol S2®, which is available from Goldschmidt Chemical Corp.;

Tego Alkanol S20 P®, which is available from Goldschmidt Chemical Corp.;

Triemulsifier 600 MS®, which is available from Tri-Tex Co.®, Inc.;

Santone 10-10-0®;

Santone 3-1-S XTR®;

Santone 3-1-SH®; and

Santone 8-1-O®;

Glycerol Esters, including but not limited to,

Agro #9 Wint SBO®, which is available from Lambent Technologies Corp.;

Ahcovel Base 700®, which is available from Uniqema;

Aldo HMS FG®, which is available from Lonza Inc.;

Aldo MLD®, which is available from Lonza Inc.;

Aldo MLD FG®, which is available from Lonza Inc.;

Aldo MO FG®, which is available from Lonza Inc.;

Aldo MS®, which is available from Lonza Inc.;

Aldo MS FG®, which is available from Lonza Inc.;

Aldo MS LG FG®, which is available from Lonza Inc.;

Aldo MSD®, which is available from Lonza Inc.;

Aldo MSD FG®, which is available from Lonza Inc.;

Aldosperse O-20 FG®, which is available from Lonza Inc.;

Aldosperse TS-20 FG®, which is available from Lonza Inc.;

Aldosperse TS-40 FG®, which is available from Lonza Inc.;

Arlacel 165®, which is available from Uniqema;

Arlacel 186®, which is available from Uniqema;

Capmul GM®, which is available from Abitec Corporation;

Capmul GMS®, which is available from Abitec Corporation;

Caprol 3GO®, which is available from Abitec Corporation;

Caprol 3GVS®, which is available from Abitec Corporation;

Caprol 6G2S®, which is available from Abitec Corporation;

Caprol 10G40®, which is available from Abitec Corporation;

Caprol 10G100®, which is available from Abitec Corporation;

Caprol ET®, which is available from Abitec Corporation;

Caprol PGE860®, which is available from Abitec Corporation;

Cerasynt 945®, which is available from International Specialty Products/IS;

Cerasynt GMS®, which is available from International Specialty Products/IS;

Cerasynt Q®, which is available from International Specialty Products/IS;

Cerasynt SD®, which is available from International Specialty Products/IS;

Cerasynt WM®, which is available from International Specialty Products/IS;

Chemsperse 14®, which is available from Chemron Corp.;

Cremophor GO-32®, which is available from BASF Corp.;

Cremophor GS11®, which is available from BASF Corp.;

Cremophor GS-32®, which is available from BASF Corp.;

Cutina KD-16®, which is available from Cognis Canada Corp.;

Dehymuls PGPH®, which is available from Cognis Corporation;

Dermol DGDIS®, which is available from Alzo International, Inc.;

Dermol DGMIS®, which is available from Alzo International, Inc.;

Dermol G-76®, which is available from Alzo International, Inc.;

Dermol G-7DI®, which is available from Alzo International, Inc.;

Dermol NGDI®, which is available from Alzo International, Inc.;

Dermolan GLH®, which is available from Alzo International, Inc.;

Drewmulse GMO®, which is available from Stepan Company;

Drewpol 3-5-M®, which is available from Stepan Company;

Durlac 100 W®, which is available from Loders Croklaan U.S.A.;

Dur-Lo®, which is available from Loders Croklaan U.S.A.;

Dynasan 118®, which is available from Sasol North America Inc.;

EC-25®, which is available from Loders Croklaan U.S.A.;

EM 40®, which is available from Keil Chemical;

Emerest 2400®, which is available from Cognis Corporation;

Emerest 2452®, which is available from Cognis Corporation;

Empilan G-26®, which is available from Huntsman LLC;

Genapol TSM®, which is available from Clariant Corporation;

Hostacerin DGI®, which is available from Clariant Corporation;

Hostacerin DGL®, which is available from Clariant Corporation;

Hostacerin DGMS®, which is available from Clariant Corporation;

Hostacerin DGS®, which is available from Clariant Corporation;

Ice No. 2®, which is available from Loders Croklaan U.S.A.;

Imwitor 742®, which is available from Sasol North America Inc.;

Imwitor 780 K®, which is available from Sasol North America Inc.;

Imwitor 960 Flakes®, which is available from Sasol North America Inc.;

Isolan GI 34®, which is available from Goldschmidt Chemical Corp.;

Isolan GO 33®, which is available from Goldschmidt Chemical Corp.;

Kemester 1000®, which is available from Crompton Corp.;

Kemester 2000®, which is available from Crompton;

Kemester 2000®, which is available from Crompton Corp.;

Kemester 6000SE®, which is available from Crompton Corp.;

Lamecreme DGE 18®, which is available from Cognis Corporation;

Lexemul 515®, which is available from Inolex Chemical Co.;

Lexemul 561®, which is available from Inolex Chemical Co.;

Lexemul AR®, which is available from Inolex Chemical Co.;

Lexemul AS®, which is available from Inolex Chemical Co.;

Lexemul GDL®, which is available from Inolex Chemical Co.;

Lexemul T®, which is available from Inolex Chemical Co.;

Lipomulse 165®, which is available from Lipo Chemicals, Inc.;

Lumulse GML K®, which is available from Lambent Technologies Corp.;

Lumulse GMO K®, which is available from Lambent Technologies Corp.;

Lumulse GMR K®, which is available from Lambent Technologies Corp.;

Lumulse GMT K®, which is available from Lambent Technologies Corp.;

Magrabar GMC®, which is available from Magrabar Chemical Corp.;

Magrabar GMO-CK®, which is available from Magrabar Chemical Corp.;

Magrabar MDG-5050®, which is available from Magrabar Chemical Corp.;

Magrabar PGO-315®, which is available from Magrabar Chemical Corp.;

Magrabar PGO-1010®, which is available from Magrabar Chemical Corp.;

Mazol 300K®, which is available from BASF Corp.;

Mazol GMO-K®, which is available from BASF Corp.;

Mazol GMS-K®, which is available from BASF Corp.;

Mazol PG031-K®, which is available from BASF Corp.;

Miglyol 812®, which is available from Sasol North America Inc.;

Norfox 165C®, which is available from Norman, Fox & Co.;

Schercemol GMIS®, which is available from Noveon®, Inc.;

Tegin®, which is available from Goldschmidt Chemical Corp.;

Tegin 4100 Pellets®, which is available from Goldschmidt Chemical Corp.;

Tegin M Pellets®, which is available from Goldschmidt Chemical Corp.;

Tegin OV®, which is available from Goldschmidt Chemical Corp.;

Teginacid H®, which is available from Goldschmidt Chemical Corp.;

Tego Cosmo P813®, which is available from Goldschmidt Chemical Corp.;

Wickenol 535®, which is available from Alzo International, Inc.;

Witconol 14®, which is available from Akzo Nobel Surface Chemistry LLC;

Witconol 14®, which is available from Crompton Corp.;

Witconol 14F®, which is available from Crompton Corp.;

Witconol 18L®, which is available from Crompton Corp.;

Witconol GOT®, which is available from Crompton Corp.;

Witconol MST®, which is available from Crompton Corp.;

Witconol RHT®, which is available from Crompton Corp.;

Alkamuls 400-DO®;

Dur-Em 114®;

Dur-Em 117®;

Dur-Em 204®;

Dur-Em 207®;

Dur-Em 207E®;

Glicepol 160®;

Glicepol 560®;

Glicepol GMS 20®;

Polyaldo 10-2-P®;

Polyaldo DGDO®;

Polyaldo HGDS®;

Polyaldo TGMS®;

Surfonic E400-MO®; and

Tegin®;

Glycol Esters, including but not limited to,

Alkamuls 600 DO®, which is available from Rhodia, Inc.;

Alkamuls SEG®, which is available from Rhodia, Inc.;

Atlas EM-2®, which is available from Atlas Refinery Inc.;

Cerasynt IP®, which is available from International Specialty Products/IS;

Cerasynt M®, which is available from International Specialty Products/IS;

Cerasynt MN®, which is available from International Specialty Products/IS;

Cerasynt PA®, which is available from International Specialty Products/IS;

Chemsperse EGDS®, which is available from Chemron Corp.;

Chemsperse EGMS®, which is available from Chemron Corp.;

Colonial Monolaurin®, which is available from Colonial Chemical Co.;

DeMuls SGE-95®, which is available from DeForest Enterprises, Inc.;

Eccoterge 200®, which is available from Eastern Color & Chemical Co.;

Emerest 2380®, which is available from Cognis Corporation;

Ethox 2610®, which is available from Ethox Chemicals, LLC;

Ethox DO-9®, which is available from Ethox Chemicals, LLC;

Ethox DO-14®, which is available from Ethox Chemicals, LLC;

Ethox SO-9®, which is available from Ethox Chemicals, LLC;

Fizul MD-318®, which is available from Finetex Inc.;

Genapol EGDS-VHP®, which is available from Clariant Corporation;

Genapol TS Powder®, which is available from Clariant Corporation;

Hostacerin WO®, which is available from Clariant Corporation;

Inversol 140®, which is available from Keil Chemical;

Kemester 104®, which is available from Crompton Corp.;

Kemester 205®, which is available from Crompton Corp.;

Kemester 226®, which is available from Crompton Corp.;

Kemester 5221SE®, which is available from Crompton Corp.;

Kemester EGDS®, which is available from Crompton Corp.;

Lexemul EGDS®, which is available from Inolex Chemical Co.;

Lexemul EGMS®, which is available from Inolex Chemical Co.;

Lexemul P®, which is available from Inolex Chemical Co.;

Lipo DGLS®, Self-Emulsifying®, which is available from Lipo Chemicals, Inc.;

Lipo EGDS®, which is available from Lipo Chemicals, Inc.;

Lipo PGMS®, which is available from Lipo Chemicals, Inc.;

Liposorb S-4®, which is available from Lipo Chemicals, Inc.;

Liposorb TO-20®, which is available from Lipo Chemicals, Inc.;

Lumulse PGO®, which is available from Lambent Technologies Corp.;

Mackester EGDS®, which is available from The McIntyre Group;

Mackester EGMS®, which is available from The McIntyre Group;

Mackester GSTP®, which is available from The McIntyre Group;

Mackester Series®, which is available from The McIntyre Group;

Magrabar PDG-50®, which is available from Magrabar Chemical Corp.;

Mapeg 6000 DS®, which is available from BASF Corp.;

Marlowet 4702®, which is available from Sasol North America Inc.;

Monalube 305®, which is available from Uniqema;

Monalube 310®, which is available from Uniqema;

Monalube 315®, which is available from Uniqema;

Monalube 320®, which is available from Uniqema;

Monalube 325®, which is available from Uniqema;

Monalube 330®, which is available from Uniqema;

Naturechem PGHS®, which is available from CasChem®, Inc.;

Polycastorol PLO-840®, which is available from Magrabar Chemical Corp.;

Polytex 10M®, which is available from Lipo Chemicals, Inc.;

Ritasynt IP®, which is available from RITA Corp.;

Ross Chem PEG 600 DT®, which is available from Lubrizol Foam Control Additives;

Schercemol PGMS®, which is available from Noveon®, Inc.;

Sponto H-44C®, which is available from Crompton Corp.;

Tegin G®, which is available from Goldschmidt Chemical Corp.;

Witbreak DGE-182®, which is available from Akzo Nobel Surface Chemistry LLC;

Witbreak DGE-182®, which is available from Crompton Corp.;

Witbreak DRA-21®, which is available from Akzo Nobel Surface Chemistry LLC;

Witbreak DRA-21®, which is available from Crompton Corp.;

Witbreak DRA-50®, which is available from Akzo Nobel Surface Chemistry LLC;

Witbreak DRA-50®, which is available from Crompton Corp.;

Witconol F26-46®, which is available from Crompton Corp.;

Witconol H-32®, which is available from Akzo Nobel Surface Chemistry LLC;

Witconol H-33®, which is available from Akzo Nobel Surface Chemistry LLC;

Witconol H-35A®, which is available from Crompton Corp.;

Witconol RHP®, which is available from Crompton Corp.;

Ethox DO-14®;

Ethox DO-9®;

Pegosperse 100-L®;

Pegosperse 100-O®;

Pegosperse 100-S®;

Pegosperse 1500-MS®;

Pegosperse 1750-MS®;

Pegosperse 200 DL®;

Pegosperse 200-ML®;

Pegosperse 400-DL®;

Pegosperse 400-DO®;

Pegosperse 400-DS®;

Pegosperse 400-ML®;

Pegosperse 400-MO®;

Pegosperse 400-MOT®;

Pegosperse 400-MS®;

Pegosperse 50-DS®;

Pegosperse 50-MS®;

Pegosperse 600-ML®;

Pegosperse 600-MS®;

Protegin V®;

Protegin W®;

Protegin WX®; and

Protegin XV®;

Lanolin-based Derivatives, including but not limited to,

Amerchol CAB®, which is available from Amerchol Corp.;

Amerchol L-101®, which is available from Amerchol Corp.;

Amerlate LFA-LO®, which is available from Amerchol Corp.;

Amerlate P®, which is available from Amerchol Corp.;

Barre Common Degras®, which is available from RITA Corp.;

Cholesterol NF®, which is available from Croda Inc.;

Crodalan AWS®, which is available from Croda Inc.;

Crodalan LA®, which is available from Croda Inc.;

Emery 1650®, which is available from Cognis Canada Corp.;

Emery 1650®, which is available from Cognis Corporation;

Emery 1740®, which is available from Cognis Canada Corp.;

Emery 1740®, which is available from Cognis Corporation;

Forlan 500®, which is available from RITA Corp.;

Forlan L®, which is available from RITA Corp.;

Laneto 50®, which is available from RITA Corp.;

Laneto 100®, which is available from RITA Corp.;

Laneto AWS®, which is available from RITA Corp.;

Lanfrax 1776®, which is available from Cognis Canada Corp.;

Lanfrax 1776®, which is available from Cognis Corporation;

Lanogel 21®, which is available from Amerchol Corp.;

Lipolan®, which is available from Lipo Chemicals, Inc.;

Lipolan 31®, which is available from Lipo Chemicals, Inc.;

OHlan®, which is available from Amerchol Corp.;

Polychol 5®, which is available from Croda Inc.;

Polychol 15®, which is available from Croda Inc.;

Ritacetyl®, which is available from RITA Corp.;

Ritachol®, which is available from RITA Corp.;

Ritahydrox®, which is available from RITA Corp.;

Ritalafa®, which is available from RITA Corp.;

Ritalan®, which is available from RITA Corp.;

Ritalan AWS®, which is available from RITA Corp.;

Ritalan C®, which is available from RITA Corp.;

Ritawax®, which is available from RITA Corp.;

Ritawax AEO®, which is available from RITA Corp.;

Ritawax ALA®, which is available from RITA Corp.;

Solan/Solan 50/Super Solan®, which is available from Croda Inc.;

Super Hartolan/Hartolan®, which is available from Croda Inc.;

Supersat AWS-4®, which is available from RITA Corp.; and

Supersat AWS-24®, which is available from RITA Corp.;

Lecithin and Lecithin Derivatives, including but not limited to,

Alcolec®, which is available from American Lecithin Co.;

Lecithin®, which is available from Archer Daniels Midland Company;

Lexin K®, which is available from American Lecithin Co.; and

Natipide®, which is available from American Lecithin Co.;

Lignin and Lignin Derivatives, including but not limited to,

Diwatex XP 9®, which is available from Borregaard Lignotech USA Inc.;

Dynasperse LCD®, which is available from Borregaard Lignotech USA Inc.;

Indulin SAL®, which is available from MeadWestvaco Corp.;

Indulin W-1®, which is available from MeadWestvaco Corp.;

Indulin W-5®, which is available from MeadWestvaco Corp.;

Lignosol FTA®, which is available from Borregaard Lignotech USA Inc.;

Lignosol SFX-65®, which is available from Borregaard Lignotech USA Inc.;

Marasperse 52 CP®, which is available from Borregaard Lignotech USA Inc.;

Marasperse AG®, which is available from Borregaard Lignotech USA Inc.;

Marasperse CBOS-4®, which is available from Borregaard Lignotech USA Inc.; and

Ufoxane 2®, which is available from Borregaard Lignotech USA Inc.;

Methyl Esters, including but not limited to,

E.B. Cleaner AK®, which is available from Eastern Color & Chemical Co.;

Oleocal ME-70®, which is available from Lambent Technologies Corp.;

Oleocal ME-92®, which is available from Lambent Technologies Corp.;

Oleocal ME-112®, which is available from Lambent Technologies Corp.; and

Oleocal ME-130®, which is available from Lambent Technologies Corp.;

Monoglycerides and Derivatives, including but not limited to,

Dynacet 211®, which is available from Sasol North America Inc.;

Hetsorb S-20®, which is available from Global-Seven, Inc.;

Imwitor 191®, which is available from Sasol North America Inc.;

Imwitor 370®, which is available from Sasol North America Inc.;

Imwitor 375®, which is available from Sasol North America Inc.;

Imwitor 900®, which is available from Sasol North America Inc.;

Imwitor 945®, which is available from Sasol North America Inc.;

Imwitor 2020®, which is available from Sasol North America Inc.;

Kemester 5500®, which is available from Crompton Corp.;

Kemester 6000®, which is available from Crompton Corp.;

Magrabar GMC®, which is available from Magrabar Chemical Corp.;

Magrabar GMO-CK®, which is available from Magrabar Chemical Corp.;

Magrabar GPC-10®, which is available from Magrabar Chemical Corp.;

Magrabar MDG-5050®, which is available from Magrabar Chemical Corp.;

Monalube 335®, which is available from Uniqema;

Monoglycerides Glyceryl Monestearate Archer Daniels Midland Company;

Rita GMS®, which is available from RITA Corp.;

Ritamulse SCG®, which is available from RITA Corp.;

Softigen 701®, which is available from Sasol North America Inc.; and

Tally 100 Plus®, which is available from Loders Croklaan U.S.A.;

Polyethylene Glycols, including but not limited to,

Emulgade PL 68/50®, which is available from Cognis Corporation;

Lumulse PEG®, which is available from Lambent Technologies Corp.;

Rhodasurf PEG-400®, which is available from Rhodia, Inc.;

Rhodasurf PEG-600®, which is available from Rhodia, Inc.; and

Witconol PEG-400®, which is available from Akzo Nobel Surface Chemistry LLC;

Polymeric Surfactants, including but not limited to,

Acritamer PNC-EG®, which is available from RITA Corp.;

Ag-Rho DEP-775®, which is available from Rhodia, Inc.;

APG 325N Glycoside®, which is available from Cognis Corporation;

Aristoflex AVC®, which is available from Clariant Corporation;

Aristoflex HMB®, which is available from Clariant Corporation;

Burco NPS-225®, which is available from Burlington Chemical Co.®, Inc.;

Burco NPS-816®, which is available from Burlington Chemical Co.®, Inc.;

Chemccinate 5603®, which is available from Chemron Corp.;

Cosmedia Guar C-261N®, which is available from Cognis Corporation;

Gantrez S-95®, which is available from International Specialty Products/IS;

Glucopon 220 UP®, which is available from Cognis Corporation;

Glucopon 225 DK®, which is available from Cognis Corporation;

Glucopon 425 N®, which is available from Cognis Corporation;

Glucopon 600 UP®, which is available from Cognis Corporation;

Glucopon 625 UP®, which is available from Cognis Corporation;

Pemulen 1621®, which is available from Noveon®, Inc.;

Pemulen 1622®, which is available from Noveon®, Inc.;

Pemulen TR-1®, which is available from Noveon®, Inc.;

Pemulen TR-2®, which is available from Noveon®, Inc.;

Plantacare 818®, which is available from Cognis Corporation;

Plantapon LGC Sorb®, which is available from Cognis Corporation;

Plantaren 1200N®, which is available from Cognis Corporation;

Plantaren 2000N®, which is available from Cognis Corporation;

Viscolam AT 64®, which is available from RITA Corp.;

Viscolam AT 64P®, which is available from RITA Corp.;

Viscolam AT 100®, which is available from RITA Corp.;

Viscolam MAC 7®, which is available from RITA Corp.;

Viscolam SMC 20®, which is available from RITA Corp.;

Witbreak RTC-323®, which is available from Crompton Corp.; and

WSI 3700®, which is available from Jacam Chemicals, L.L.C.;

Propoxylated & Ethoxylated Fatty Acids, Alcohols, or Alkyl Phenols, including but not limited to,

Antarox AA-60®, which is available from Rhodia, Inc.;

Antarox LF-224®, which is available from Rhodia, Inc.;

Burcomul DFE-45®, which is available from Burlington Chemical Co.®, Inc.;

Burcoterge LFE-1000®, which is available from Burlington Chemical Co.®, Inc.;

Chemal LF-25B®, which is available from Chemax Performance Solutions;

Chemal LF-40B®, which is available from Chemax Performance Solutions;

Dehypon LS-36®, which is available from Cognis Canada Corp.;

Dehypon LS-36®, which is available from Cognis Corporation;

Dehypon LS-54®, which is available from Cognis Canada Corp.;

Dehypon LS-54®, which is available from Cognis Corporation;

Delonic 100 VLF®, which is available from DeForest Enterprises, Inc.;

Delonic LF-60 MOD®, which is available from DeForest Enterprises, Inc.;

Empiderm B®, which is available from Huntsman LLC;

Ethylan 1206®, which is available from Akzo Nobel Surface Chemistry LLC;

Ethylan NS-500K®, which is available from Akzo Nobel Surface Chemistry LLC;

Ethylan NS-500LQ®, which is available from Akzo Nobel Surface Chemistry LLC;

Genapol 1392®, which is available from Clariant Corporation;

Genapol 2317®, which is available from Clariant Corporation;

Genapol 26EP710®, which is available from Clariant Corporation;

Genapol EP 1022®, which is available from Clariant Corporation;

Genapol EP 1024®, which is available from Clariant Corporation;

Genapol EP 6068®, which is available from Clariant Corporation;

Genapol NP915®, which is available from Clariant Corporation;

Kieralon MFB®, which is available from BASF Corp.;

Lumisolve CSA-80 V®, which is available from Lambent Technologies Corp.;

Marlowet 5001®, which is available from Sasol North America Inc.;

Marlox FK 64®, which is available from Sasol North America Inc.;

Marlox MO 124®, which is available from Sasol North America Inc.;

Marlox S 58®, which is available from Sasol North America Inc.;

Nonatell 1003®, which is available from Tomah Products®, Inc.;

Nonatell 1038®, which is available from Tomah Products®, Inc.;

Nonatell 1052®, which is available from Tomah Products®, Inc.;

Nonatell 1061®, which is available from Tomah Products®, Inc.;

Nonatell 1075®, which is available from Tomah Products®, Inc.;

Nonatell 1088®, which is available from Tomah Products®, Inc.;

Nonatell 1123®, which is available from Tomah Products®, Inc.;

Nonatell 1153®, which is available from Tomah Products®, Inc.;

Nonatell 1161®, which is available from Tomah Products®, Inc.;

Nonatell 1172®, which is available from Tomah Products®, Inc.;

Nonatell 1181®, which is available from Tomah Products®, Inc.;

Norfox 36®, which is available from Norman, Fox & Co.;

Procetyl AWS®, which is available from Croda Inc.;

Sandoxylate SX 412®Liquid, which is available from Clariant Corporation;

Sandoxylate SX 418®, which is available from Clariant Corporation;

Surfonic JL-80X®, which is available from Huntsman LLC;

Surfonic JL-80X-B1®, which is available from Huntsman LLC;

Surfonic L4-29X®, which is available from Huntsman LLC;

Surfonic LF®, which is available from Huntsman LLC;

T-Det A826®, which is available from Harcros Chemicals Inc.;

T-Det LF-416®, which is available from Harcros Chemicals Inc.;

Tergitol Min-Foam 1X®, which is available from Dow Chemical Company;

Tergitol Min-Foam 2X®, which is available from Dow Chemical Company;

Triton CF-21®, which is available from Dow Chemical Company;

Triton CF-76®, which is available from Dow Chemical Company;

Triton XL-80N®, which is available from Dow Chemical Company;

Witconol NS-98®, which is available from Akzo Nobel Surface Chemistry LLC;

Witconol NS-108LQ®, which is available from Akzo Nobel Surface Chemistry LLC;

Witconol NS-145®, which is available from Akzo Nobel Surface Chemistry LLC; and

Witconol NS-179®, which is available from Akzo Nobel Surface Chemistry LLC;

Chemal LFL-17®;

Chemal LFL-19®;

Chemal LFL-28 C®;

Chemal LFL-47®;

Delonic LF-EP-15®;

Delonic LF-EP-18®;

Delonic LF-EP-20®;

Delonic LF-EP-25®;

Delonic LF-EP-30®;

Delonic LF-EP-35®;

Delonic LF-EP-40®;

Delonic LF-EP-61®;

Soprophor 796/P®;

Protein-based Surfactants, including but not limited to,

AminoFoam W®, which is available from Croda Inc.;

Amiter LGOD-2®, which is available from Ajinomoto USA, Inc.;

Amiter LGS-2®, which is available from Ajinomoto USA, Inc.;

Amiter LGS-5®, which is available from Ajinomoto USA, Inc.;

Lamepon S®, which is available from Cognis Canada Corp.;

Lamepon S®, which is available from Cognis Corporation;

Maypon 4C®, which is available from Inolex Chemical Co.;

May-Tein C®, which is available from Maybrook, Inc.;

May-Tein CT®, which is available from Maybrook, Inc.;

May-Tein KTS®, which is available from Maybrook, Inc.;

May-Tein SY®, which is available from Maybrook, Inc.;

Plantapon S®, which is available from Cognis Corporation;

Proteol APL®, which is available from Seppic Inc.;

Proteol OAT®, which is available from Seppic Inc.;

Pyroter CPI-40®, which is available from Ajinomoto USA, Inc.;

Pyroter GPI-25®, which is available from Ajinomoto USA, Inc.;

Supro-Tein S®, which is available from Maybrook, Inc.; and

Supro-Tein V®, which is available from Maybrook, Inc.;

Sarcosine Derivatives, including but not limited to,

Crodasinic LS-30®, which is available from Croda Inc.;

Vanseal CS®, which is available from R. T. Vanderbilt Co. Inc.;

Vanseal LS®, which is available from R. T. Vanderbilt Co. Inc.;

Vanseal MS®, which is available from R. T. Vanderbilt Co. Inc.;

Vanseal NACS-30®, which is available from R. T. Vanderbilt Co. Inc.;

Vanseal NALS-95®, which is available from R. T. Vanderbilt Co. Inc.; and

Vanseal OS®, which is available from R. T. Vanderbilt Co. Inc.;

Silicone-based Surfactants, including but not limited to,

Abil-B-9950®, which is available from Goldschmidt Chemical Corp.;

Abil Care 85®, which is available from Goldschmidt Chemical Corp.;

Abil EM 90®, which is available from Goldschmidt Chemical Corp.;

Abil EM 97®, which is available from Goldschmidt Chemical Corp.;

Abil WE-09®, which is available from Goldschmidt Chemical Corp.;

Dow Corning 1248 Fluid®, which is available from Dow Corning Corp.;

Dow Corning 3225C®Formulation Aid, which is available from Dow Corning Corp.;

Dow Corning 5200®Formulation Aid, which is available from Dow Corning Corp.;

Dow Corning Q4-3667®Fluid, which is available from Dow Corning Corp.;

Monasil PCA®, which is available from Uniqema;

Monasil PDM®, which is available from Uniqema;

Monasil PLN®, which is available from Uniqema;

Polyderm PPI-SI-WS®, which is available from Alzo International, Inc.;

Troysol 380W®, which is available from Troy Corporation;

Troysol S366®, which is available from Troy Corporation;

Abil-B-88183®;

Abil-B-8832®;

Abil-B-8843®;

Abil-B-8851®;

Abil-B-8852®;

Abil-B-8863®;

Dow Corning 190 Surfactant®;

Dow Corning 193 Surfactant®;

Dow Corning 5103 Surfactant®;

Dow Corning FF400 Fluid®;

Silwet L-7001®;

Silwet L-7002®;

Silwet L-7087®;

Silwet L-720®;

Silwet L-7200®;

Silwet L-7210®;

Silwet L-722®;

Silwet L-7220®;

Silwet L-7230®;

Silwet L-7500®;

Silwet L-7600®;

Silwet L-7602®;

Silwet L-7604®;

Silwet L-7605®;

Silwet L-7607®;

Silwet L-7608®;

Silwet L-7622®;

Silwet L-7657®; and

Silwet L-77®;

Sorbitan Derivatives, including but not limited to,

Alkamuls SML®, which is available from Rhodia, Inc.;

Alkamuls SMO®, which is available from Rhodia, Inc.;

Alkamuls STO®, which is available from Rhodia, Inc.;

Arlacel 20®, which is available from Uniqema;

Arlacel 40®, which is available from Uniqema;

Arlacel 60®, which is available from Uniqema;

Arlacel 80®, which is available from Uniqema;

Arlacel C®, which is available from Uniqema;

Armul 21®, which is available from Crompton Corp.;

Atlox 80®, which is available from Uniqema;

Atlox 847®, which is available from Uniqema;

Atlox 1045A®, which is available from Uniqema;

Canarcel 20®, which is available from Canamex Quimicos S.A de C.v;

Canarcel 60®, which is available from Canamex Quimicos S.A de C.v;

Canarcel 80®, which is available from Canamex Quimicos S.A de C.v;

Canarcel TW 20®, which is available from Canamex Quimicos S.A de C.v;

Canarcel TW 60®, which is available from Canamex Quimicos S.A de C.v;

Canarcel TW 80®, which is available from Canamex Quimicos S.A de C.v;

Coladet BSB-P®, which is available from Colonial Chemical Co.;

Customulse O-20®, which is available from Custom Ingredients, Inc.;

Dehymuls E®, which is available from Cognis Canada Corp.;

DeSotan SMO®, which is available from Crompton Corp.;

DeSotan SMO-20®, which is available from Crompton Corp.;

DeSotan SMT®, which is available from Crompton Corp.;

DeSotan SMT-20®, which is available from Crompton Corp.;

Durfax 60®, which is available from Loders Croklaan U.S.A.;

Durfax 65®, which is available from Loders Croklaan U.S.A.;

Durfax 80®, which is available from Loders Croklaan U.S.A.;

Durtan 60®, which is available from Loders Croklaan U.S.A.;

Durtan 65®, which is available from Loders Croklaan U.S.A.;

Liposorb L®, which is available from Lipo Chemicals, Inc.;

Liposorb L-10®, which is available from Lipo Chemicals, Inc.;

Liposorb L-20®, which is available from Lipo Chemicals, Inc.;

Liposorb O®, which is available from Lipo Chemicals, Inc.;

Liposorb O-20®, which is available from Lipo Chemicals, Inc.;

Liposorb P®, which is available from Lipo Chemicals, Inc.;

Liposorb P-20®, which is available from Lipo Chemicals, Inc.;

Liposorb S®, which is available from Lipo Chemicals, Inc.;

Liposorb S-20®, which is available from Lipo Chemicals, Inc.;

Liposorb SQO®, which is available from Lipo Chemicals, Inc.;

Liposorb TO®, which is available from Lipo Chemicals, Inc.;

Liposorb TS®, which is available from Lipo Chemicals, Inc.;

Liposorb TS-20®, which is available from Lipo Chemicals, Inc.;

Lumisorb PS®, which is available from Lambent Technologies Corp.;

Lumisorb SMO (T)®, which is available from Lambent Technologies Corp.;

Lumisorb SMS K®, which is available from Lambent Technologies Corp.;

Lumisorb SSO®, which is available from Lambent Technologies Corp.;

Lumisorb STS K®, which is available from Lambent Technologies Corp.;

Lumisorb STT®, which is available from Lambent Technologies Corp.;

Magrabar SMO®, which is available from Magrabar Chemical Corp.;

Magrabar SMO-VEG®, which is available from Magrabar Chemical Corp.;

Magrabar SMT®, which is available from Magrabar Chemical Corp.;

Magrabar STO®, which is available from Magrabar Chemical Corp.;

Miracare BC-27®, which is available from Rhodia, Inc.;

Ritabate 20®, which is available from RITA Corp.;

Ritabate 40®, which is available from RITA Corp.;

Ritabate 60®, which is available from RITA Corp.;

Ritabate 80®, which is available from RITA Corp.;

T-Maz®, which is available from BASF Corp.;

Tego SML®, which is available from Goldschmidt Chemical Corp.;

Tego SML 20®, which is available from Goldschmidt Chemical Corp.;

Tego SMO 80 V®, which is available from Goldschmidt Chemical Corp.;

Tego SMO V®, which is available from Goldschmidt Chemical Corp.;

Tego SMS®, which is available from Goldschmidt Chemical Corp.;

Tego STO V®, which is available from Goldschmidt Chemical Corp.;

Tween 21®, which is available from Uniqema;

Tween 40®, which is available from Uniqema;

Tween 60®, which is available from Uniqema;

Tween 60 K®, which is available from Uniqema;

Tween 61®, which is available from Uniqema;

Tween 65®, which is available from Uniqema;

Tween 80®, which is available from Uniqema;

Tween 80 K®, which is available from Uniqema;

Tween 81®, which is available from Uniqema;

Tween 85®, which is available from Uniqema;

Alkamuls PSML-20®;

Alkamuls PSMO-20®;

Alkamuls PSTO-20®;

Cremophor PS150®;

Cremophor PS20®;

Cremophor PS28®;

Cremophor PS60®;

Cremophor PS80®;

Crill 3®;

Crill 4®;

Crill 6®;

Crillet 3®;

Crillet 4®;

Crillet 6®;

Glycomul L®;

Glycomul O®;

Glycomul S®;

Glycomul TO®;

Glycomul TS®;

Hetsorb L-20®;

Hetsorb L-4®;

Hetsorb L-80-72%®;

Hetsorb O-20®;

Hetsorb O-5®;

Hetsorb TO-20®;

Lumisorb PSML-20 K®;

Lumisorb PSML-20 NF®;

Lumisorb PSMO-20 K®;

Lumisorb PSMO-5 K®;

Lumisorb PSMS-20 K®;

Lumisorb PSTS-20 K®;

Lumisorb PSTT-20 K®;

Sorbax PML-20®;

Sorbax PMO-20®;

Sorbax PMO-5®;

Sorbax PMP-20®;

Sorbax PMS-20®;

Sorbax PTO-20®;

Sorbax PTS-20®;

Sorbax SML®;

Sorbax SMO®;

Sorbax SMP®;

Sorbax SMS®;

Sorbax STO®;

Sorbax STS®;

Span 20®;

Span 40®;

Span 60®;

Span 60K®;

Span 65®;

Span 80®;

Span 85®;

T-Maz 20®;

T-Maz 28®;

T-Maz 60K®;

T-Maz 65K®;

T-Maz 80®;

T-Maz 80K®;

T-Maz 81®;

T-Maz 85®; and

T-Maz 90®;

Sucrose and Glucose Esters and Derivatives, including but not limited to,

DeSulf GOS-P-60WCG®, which is available from DeForest Enterprises, Inc.;

Glucam E-20 Distearate®, which is available from Amerchol Corp.;

Glucamate DOE-120®, which is available from Amerchol Corp.;

Glucamate SSE-20®, which is available from Amerchol Corp.;

Glucate DO®, which is available from Amerchol Corp.;

Glucate SS®, which is available from Amerchol Corp.;

Glucopon 425 UP®, which is available from Cognis Corporation;

Isolan IS®, which is available from Goldschmidt Chemical Corp.;

Mazon 40®, which is available from BASF Corp.;

Montanov 82®, which is available from Seppic Inc.;

Montanov 202®, which is available from Seppic Inc.;

Montanov S®, which is available from Seppic Inc.;

Rheozan®, which is available from Rhodia, Inc.;

Simulsol AS 48®, which is available from Seppic Inc.;

Simulsol SL 4®, which is available from Seppic Inc.;

Simulsol SL 10®, which is available from Seppic Inc.;

Simulsol SL 11W®, which is available from Seppic Inc.;

Simulsol SL 55®, which is available from Seppic Inc.;

Suga Nate 100 and 160®, which is available from Colonial Chemical Co.;

Tego Care 450®, which is available from Goldschmidt Chemical Corp.;

Tego Care CG 90®, which is available from Goldschmidt Chemical Corp.;

Tego Care PS®, which is available from Goldschmidt Chemical Corp.;

Tegosoft PSE 141 G®, which is available from Goldschmidt Chemical Corp.;

Tegotens G 826®, which is available from Goldschmidt Chemical Corp.;

Triton BG-10 (70%)®, which is available from Dow Chemical Company;

Triton CG-110 (60%)®, which is available from Dow Chemical Company;

Wickenol 545®, which is available from Alzo International, Inc;

Tego Care 150®;

Tego Care 215®; and

Triton CG-110 (60%)®.

Pluronic from BASF is another useful type of surfactant.

One embodiment comprises a sorbitan ester.

Another embodiment comprises Polysorbate 20.

Another embodiment comprises Polysorbate 40.

Another embodiment comprises Polysorbate 60.

Another embodiment comprises Polysorbate 80.

Another embodiment comprises a stearate.

Another embodiment comprises glyceryl stearate.

Another embodiment comprises isopropyl stearate.

Another embodiment comprises polyoxyl stearate.

Another embodiment comprises propylene glycol stearate.

Another embodiment comprises sucrose stearate.

Another embodiment comprises polyethylene glycol.

Another embodiment comprises polyethylene oxide.

Another embodiment comprises polypropylene oxide.

Another embodiment comprises a polyethylene oxide-polypropylene oxide copolymer.

Another embodiment comprises an alcohol ethoxylate.

Another embodiment comprises an alkylphenol ethoxylate.

Another embodiment comprises an alkyl glycoside.

Another embodiment comprises alkyl polyglycoside.

Another embodiment comprises a fatty alcohol.

Another embodiment comprises hydroxypropylmethyl cellulose.

Another embodiment comprises carboxymethyl cellulose.

Another embodiment comprises a polyacrylic acid.

Another embodiment comprises a Carbomer.

Another embodiment comprises a phosphalipid.

Another embodiment comprises phosphatidyl chloline.

Another embodiment comprises phosphatidyl serine.

Another embodiment comprises cyclosporin A, an oil having a specific gravity from 0.95 to 1.07, and a sorbitan ester.

Another embodiment comprises cyclosporin A, Anise oil, and a sorbitan ester.

Another embodiment comprises cyclosporin A, Castor oil, and a sorbitan ester.

Another embodiment comprises cyclosporin A, Clove oil, and a sorbitan ester.

Another embodiment comprises cyclosporin A, Cassia oil, and a sorbitan ester.

Another embodiment comprises cyclosporin A, Cinnamon oil, and a sorbitan ester.

Another embodiment comprises cyclosporin A, an oil having a specific gravity from 0.90 to 0.95, and a sorbitan ester.

Another embodiment comprises cyclosporin A, Almond oil, and a sorbitan ester.

Another embodiment comprises cyclosporin A, Corn oil, and a sorbitan ester.

Another embodiment comprises cyclosporin A, Arachis oil, and a sorbitan ester.

Another embodiment comprises cyclosporin A, Cottonseed oil, and a sorbitan ester.

Another embodiment comprises cyclosporin A, Safflower oil, and a sorbitan ester.

Another embodiment comprises cyclosporin A, Maize oil, and a sorbitan ester.

Another embodiment comprises cyclosporin A, Linseed oil, and a sorbitan ester.

Another embodiment comprises cyclosporin A, Rapeseed oil, and a sorbitan ester.

Another embodiment comprises cyclosporin A, Soybean oil, and a sorbitan ester.

Another embodiment comprises cyclosporin A, Olive oil, and a sorbitan ester.

Another embodiment comprises cyclosporin A, Caraway oil, and a sorbitan ester.

Another embodiment comprises cyclosporin A, Rosemary oil, and a sorbitan ester.

Another embodiment comprises cyclosporin A, Peanut oil, and a sorbitan ester.

Another embodiment comprises cyclosporin A, Peppermint oil, and a sorbitan ester.

Another embodiment comprises cyclosporin A, Sunflower oil, and a sorbitan ester.

Another embodiment comprises cyclosporin A, Eucalyptus oil, and a sorbitan ester.

Another embodiment comprises cyclosporin A, Sesame oil, and a sorbitan ester.

Another embodiment comprises cyclosporin A, an oil having a specific gravity below 0.9, and a sorbitan ester.

Another embodiment comprises cyclosporin A, Mineral oil, and a sorbitan ester.

Another embodiment comprises cyclosporin A, Coriander oil, and a sorbitan ester.

Another embodiment comprises cyclosporin A, Lavender oil, and a sorbitan ester.

Another embodiment comprises cyclosporin A, Citronella oil, and a sorbitan ester.

Another embodiment comprises cyclosporin A, Juniper oil, and a sorbitan ester.

Another embodiment comprises cyclosporin A, Lemon oil, and a sorbitan ester.

Another embodiment comprises cyclosporin A, Orange oil, and a sorbitan ester.

Another embodiment comprises cyclosporin A, Clary sage oil, and a sorbitan ester.

Another embodiment comprises cyclosporin A, Nutmeg oil, and a sorbitan ester.

Another embodiment comprises cyclosporin A, Tea tree oil, and a sorbitan ester.

Another embodiment comprises cyclosporin A, an oil having a specific gravity from 0.95 to 1.07, and Polysorbate 80.

Another embodiment comprises cyclosporin A, Anise oil, and Polysorbate 80.

Another embodiment comprises cyclosporin A, Clove oil, and Polysorbate 80.

Another embodiment comprises cyclosporin A, Cassia oil, and Polysorbate 80.

Another embodiment comprises cyclosporin A, Cinnamon oil, and Polysorbate 80.

Another embodiment comprises cyclosporin A, an oil having a specific gravity from 0.90 to 0.95, and Polysorbate 80.

Another embodiment comprises cyclosporin A, Almond oil, and Polysorbate 80.

Another embodiment comprises cyclosporin A, Corn oil, and Polysorbate 80.

Another embodiment comprises cyclosporin A, Arachis oil, and Polysorbate 80.

Another embodiment comprises cyclosporin A, Cottonseed oil, and Polysorbate 80.

Another embodiment comprises cyclosporin A, Safflower oil, and Polysorbate 80.

Another embodiment comprises cyclosporin A, Maize oil, and Polysorbate 80.

Another embodiment comprises cyclosporin A, Linseed oil, and Polysorbate 80.

Another embodiment comprises cyclosporin A, Rapeseed oil, and Polysorbate 80.

Another embodiment comprises cyclosporin A, Soybean oil, and Polysorbate 80.

Another embodiment comprises cyclosporin A, Olive oil, and Polysorbate 80.

Another embodiment comprises cyclosporin A, Caraway oil, and Polysorbate 80.

Another embodiment comprises cyclosporin A, Rosemary oil, and Polysorbate 80.

Another embodiment comprises cyclosporin A, Peanut oil, and Polysorbate 80.

Another embodiment comprises cyclosporin A, Peppermint oil, and Polysorbate 80.

Another embodiment comprises cyclosporin A, Sunflower oil, and Polysorbate 80.

Another embodiment comprises cyclosporin A, Eucalyptus oil, and Polysorbate 80.

Another embodiment comprises cyclosporin A, Sesame oil, and Polysorbate 80.

Another embodiment comprises cyclosporin A, an oil having a specific gravity below 0.9, and Polysorbate 80.

Another embodiment comprises cyclosporin A, Mineral oil, and Polysorbate 80.

Another embodiment comprises cyclosporin A, Coriander oil, and Polysorbate 80.

Another embodiment comprises cyclosporin A, Lavender oil, and Polysorbate 80.

Another embodiment comprises cyclosporin A, Citronella oil, and Polysorbate 80.

Another embodiment comprises cyclosporin A, Juniper oil, and Polysorbate 80.

Another embodiment comprises cyclosporin A, Lemon oil, and Polysorbate 80.

Another embodiment comprises cyclosporin A, Orange oil, and Polysorbate 80.

Another embodiment comprises cyclosporin A, Clary sage oil, and Polysorbate 80.

Another embodiment comprises cyclosporin A, Nutmeg oil, and Polysorbate 80.

Another embodiment comprises cyclosporin A, Tea tree oil, and Polysorbate 80.

Another embodiment comprises cyclosporin A, an oil having a specific gravity from 0.95 to 1.07, and Polysorbate 80.

Another embodiment comprises cyclosporin A, Anise oil, and Polysorbate 80.

Another embodiment comprises cyclosporin A, Castor oil, and Polysorbate 80.

Another embodiment comprises cyclosporin A, Clove oil, and Polysorbate 80.

Another embodiment comprises cyclosporin A, Cassia oil, and Polysorbate 80.

Another embodiment comprises cyclosporin A, Cinnamon oil, and Polysorbate 80.

Another embodiment comprises cyclosporin A, an oil having a specific gravity from 0.90 to 0.95, and Polysorbate 80.

Another embodiment comprises cyclosporin A, Almond oil, and Polysorbate 80.

Another embodiment comprises cyclosporin A, Corn oil, and Polysorbate 80.

Another embodiment comprises cyclosporin A, Arachis oil, and Polysorbate 80.

Another embodiment comprises cyclosporin A, Cottonseed oil, and Polysorbate 80.

Another embodiment comprises cyclosporin A, Safflower oil, and Polysorbate 80.

Another embodiment comprises cyclosporin A, Maize oil, and Polysorbate 80.

Another embodiment comprises cyclosporin A, Linseed oil, and Polysorbate 80.

Another embodiment comprises cyclosporin A, Rapeseed oil, and Polysorbate 80.

Another embodiment comprises cyclosporin A, Soybean oil, and Polysorbate 80.

Another embodiment comprises cyclosporin A, Olive oil, and Polysorbate 80.

Another embodiment comprises cyclosporin A, Caraway oil, and Polysorbate 80.

Another embodiment comprises cyclosporin A, Rosemary oil, and Polysorbate 80.

Another embodiment comprises cyclosporin A, Peanut oil, and Polysorbate 80.

Another embodiment comprises cyclosporin A, Peppermint oil, and Polysorbate 80.

Another embodiment comprises cyclosporin A, Sunflower oil, and Polysorbate 80.

Another embodiment comprises cyclosporin A, Eucalyptus oil, and Polysorbate 80.

Another embodiment comprises cyclosporin A, Sesame oil, and Polysorbate 80.

Another embodiment comprises cyclosporin A, an oil having a specific gravity below 0.9, and Polysorbate 80.

Another embodiment comprises cyclosporin A, Mineral oil, and Polysorbate 80.

Another embodiment comprises cyclosporin A, Coriander oil, and Polysorbate 80.

Another embodiment comprises cyclosporin A, Lavender oil, and Polysorbate 80.

Another embodiment comprises cyclosporin A, Citronella oil, and Polysorbate 80.

Another embodiment comprises cyclosporin A, Juniper oil, and Polysorbate 80.

Another embodiment comprises cyclosporin A, Lemon oil, and Polysorbate 80.

Another embodiment comprises cyclosporin A, Orange oil, and Polysorbate 80.

Another embodiment comprises cyclosporin A, Clary sage oil, and Polysorbate 80.

Another embodiment comprises cyclosporin A, Nutmeg oil, and Polysorbate 80.

Another embodiment comprises cyclosporin A, Tea tree oil, and Polysorbate 80.

Another embodiment comprises cyclosporin A, an oil having a specific gravity from 0.95 to 1.07, and Polysorbate 60.

Another embodiment comprises cyclosporin A, Anise oil, and Polysorbate 60.

Another embodiment comprises cyclosporin A, Castor oil, and Polysorbate 60.

Another embodiment comprises cyclosporin A, Clove oil, and Polysorbate 60.

Another embodiment comprises cyclosporin A, Cassia oil, and Polysorbate 60.

Another embodiment comprises cyclosporin A, Cinnamon oil, and Polysorbate 60.

Another embodiment comprises cyclosporin A, an oil having a specific gravity from 0.90 to 0.95, and Polysorbate 60.

Another embodiment comprises cyclosporin A, Almond oil, and Polysorbate 60.

Another embodiment comprises cyclosporin A, Corn oil, and Polysorbate 60.

Another embodiment comprises cyclosporin A, Arachis oil, and Polysorbate 60.

Another embodiment comprises cyclosporin A, Cottonseed oil, and Polysorbate 60.

Another embodiment comprises cyclosporin A, Safflower oil, and Polysorbate 60.

Another embodiment comprises cyclosporin A, Maize oil, and Polysorbate 60.

Another embodiment comprises cyclosporin A, Linseed oil, and Polysorbate 60.

Another embodiment comprises cyclosporin A, Rapeseed oil, and Polysorbate 60.

Another embodiment comprises cyclosporin A, Soybean oil, and Polysorbate 60.

Another embodiment comprises cyclosporin A, Olive oil, and Polysorbate 60.

Another embodiment comprises cyclosporin A, Caraway oil, and Polysorbate 60.

Another embodiment comprises cyclosporin A, Rosemary oil, and Polysorbate 60.

Another embodiment comprises cyclosporin A, Peanut oil, and Polysorbate 60.

Another embodiment comprises cyclosporin A, Peppermint oil, and Polysorbate 60.

Another embodiment comprises cyclosporin A, Sunflower oil, and Polysorbate 60.

Another embodiment comprises cyclosporin A, Eucalyptus oil, and Polysorbate 60.

Another embodiment comprises cyclosporin A, Sesame oil, and Polysorbate 60.

Another embodiment comprises cyclosporin A, an oil having a specific gravity below 0.9, and Polysorbate 60.

Another embodiment comprises cyclosporin A, Mineral oil, and Polysorbate 60.

Another embodiment comprises cyclosporin A, Coriander oil, and Polysorbate 60.

Another embodiment comprises cyclosporin A, Lavender oil, and Polysorbate 60.

Another embodiment comprises cyclosporin A, Citronella oil, and Polysorbate 60.

Another embodiment comprises cyclosporin A, Juniper oil, and Polysorbate 60.

Another embodiment comprises cyclosporin A, Lemon oil, and Polysorbate 60.

Another embodiment comprises cyclosporin A, Orange oil, and Polysorbate 60.

Another embodiment comprises cyclosporin A, Clary sage oil, and Polysorbate 60.

Another embodiment comprises cyclosporin A, Nutmeg oil, and Polysorbate 60.

Another embodiment comprises cyclosporin A, Tea tree oil, and Polysorbate 60.

Another embodiment comprises cyclosporin A, an oil having a specific gravity from 0.95 to 1.07, and Polysorbate 40.

Another embodiment comprises cyclosporin A, Anise oil, and Polysorbate 40.

Another embodiment comprises cyclosporin A, Castor oil, and Polysorbate 40.

Another embodiment comprises cyclosporin A, Clove oil, and Polysorbate 40.

Another embodiment comprises cyclosporin A, Cassia oil, and Polysorbate 40.

Another embodiment comprises cyclosporin A, Cinnamon oil, and Polysorbate 40.

Another embodiment comprises cyclosporin A, an oil having a specific gravity from 0.90 to 0.95, and Polysorbate 40.

Another embodiment comprises cyclosporin A, Almond oil, and Polysorbate 40.

Another embodiment comprises cyclosporin A, Corn oil, and Polysorbate 40.

Another embodiment comprises cyclosporin A, Arachis oil, and Polysorbate 40.

Another embodiment comprises cyclosporin A, Cottonseed oil, and Polysorbate 40.

Another embodiment comprises cyclosporin A, Safflower oil, and Polysorbate 40.

Another embodiment comprises cyclosporin A, Maize oil, and Polysorbate 40.

Another embodiment comprises cyclosporin A, Linseed oil, and Polysorbate 40.

Another embodiment comprises cyclosporin A, Rapeseed oil, and Polysorbate 40.

Another embodiment comprises cyclosporin A, Soybean oil, and Polysorbate 40.

Another embodiment comprises cyclosporin A, Olive oil, and Polysorbate 40.

Another embodiment comprises cyclosporin A, Caraway oil, and Polysorbate 40.

Another embodiment comprises cyclosporin A, Rosemary oil, and Polysorbate 40.

Another embodiment comprises cyclosporin A, Peanut oil, and Polysorbate 40.

Another embodiment comprises cyclosporin A, Peppermint oil, and Polysorbate 40.

Another embodiment comprises cyclosporin A, Sunflower oil, and Polysorbate 40.

Another embodiment comprises cyclosporin A, Eucalyptus oil, and Polysorbate 40.

Another embodiment comprises cyclosporin A, Sesame oil, and Polysorbate 40.

Another embodiment comprises cyclosporin A, an oil having a specific gravity below 0.9, and Polysorbate 40.

Another embodiment comprises cyclosporin A, Mineral oil, and Polysorbate 40.

Another embodiment comprises cyclosporin A, Coriander oil, and Polysorbate 40.

Another embodiment comprises cyclosporin A, Lavender oil, and Polysorbate 40.

Another embodiment comprises cyclosporin A, Citronella oil, and Polysorbate 40.

Another embodiment comprises cyclosporin A, Juniper oil, and Polysorbate 40.

Another embodiment comprises cyclosporin A, Lemon oil, and Polysorbate 40.

Another embodiment comprises cyclosporin A, Orange oil, and Polysorbate 40.

Another embodiment comprises cyclosporin A, Clary sage oil, and Polysorbate 40.

Another embodiment comprises cyclosporin A, Nutmeg oil, and Polysorbate 40.

Another embodiment comprises cyclosporin A, Tea tree oil, and Polysorbate 40.

Another embodiment comprises cyclosporin A, an oil having a specific gravity from 0.95 to 1.07, and Polysorbate 20.

Another embodiment comprises cyclosporin A, Anise oil, and Polysorbate 20.

Another embodiment comprises cyclosporin A, Castor oil, and Polysorbate 20.

Another embodiment comprises cyclosporin A, Clove oil, and Polysorbate 20.

Another embodiment comprises cyclosporin A, Cassia oil, and Polysorbate 20.

Another embodiment comprises cyclosporin A, Cinnamon oil, and Polysorbate 20.

Another embodiment comprises cyclosporin A, an oil having a specific gravity from 0.90 to 0.95, and Polysorbate 20.

Another embodiment comprises cyclosporin A, Almond oil, and Polysorbate 20.

Another embodiment comprises cyclosporin A, Corn oil, and Polysorbate 20.

Another embodiment comprises cyclosporin A, Arachis oil, and Polysorbate 20.

Another embodiment comprises cyclosporin A, Cottonseed oil, and Polysorbate 20.

Another embodiment comprises cyclosporin A, Safflower oil, and Polysorbate 20.

Another embodiment comprises cyclosporin A, Maize oil, and Polysorbate 20.

Another embodiment comprises cyclosporin A, Linseed oil, and Polysorbate 20.

Another embodiment comprises cyclosporin A, Rapeseed oil, and Polysorbate 20.

Another embodiment comprises cyclosporin A, Soybean oil, and Polysorbate 20.

Another embodiment comprises cyclosporin A, Olive oil, and Polysorbate 20.

Another embodiment comprises cyclosporin A, Caraway oil, and Polysorbate 20.

Another embodiment comprises cyclosporin A, Rosemary oil, and Polysorbate 20.

Another embodiment comprises cyclosporin A, Peanut oil, and Polysorbate 20.

Another embodiment comprises cyclosporin A, Peppermint oil, and Polysorbate 20.

Another embodiment comprises cyclosporin A, Sunflower oil, and Polysorbate 20.

Another embodiment comprises cyclosporin A, Eucalyptus oil, and Polysorbate 20.

Another embodiment comprises cyclosporin A, Sesame oil, and Polysorbate 20.

Another embodiment comprises cyclosporin A, an oil having a specific gravity below 0.9, and Polysorbate 20.

Another embodiment comprises cyclosporin A, Mineral oil, and Polysorbate 20.

Another embodiment comprises cyclosporin A, Coriander oil, and Polysorbate 20.

Another embodiment comprises cyclosporin A, Lavender oil, and Polysorbate 20.

Another embodiment comprises cyclosporin A, Citronella oil, and Polysorbate 20.

Another embodiment comprises cyclosporin A, Juniper oil, and Polysorbate 20.

Another embodiment comprises cyclosporin A, Lemon oil, and Polysorbate 20.

Another embodiment comprises cyclosporin A, Orange oil, and Polysorbate 20.

Another embodiment comprises cyclosporin A, Clary sage oil, and Polysorbate 20.

Another embodiment comprises cyclosporin A, Nutmeg oil, and Polysorbate 20.

Another embodiment comprises cyclosporin A, Tea tree oil, and Polysorbate 20.

Another embodiment comprises cyclosporin A, an oil having a specific gravity from 0.95 to 1.07, and a stearate surfactant.

Another embodiment comprises cyclosporin A, Anise oil, and a stearate surfactant.

Another embodiment comprises cyclosporin A, Castor oil, and a stearate surfactant.

Another embodiment comprises cyclosporin A, Clove oil, and a stearate surfactant.

Another embodiment comprises cyclosporin A, Cassia oil, and a stearate surfactant.

Another embodiment comprises cyclosporin A, Cinnamon oil, and a stearate surfactant.

Another embodiment comprises cyclosporin A, an oil having a specific gravity from 0.90 to 0.95, and a stearate surfactant.

Another embodiment comprises cyclosporin A, Almond oil, and a stearate surfactant.

Another embodiment comprises cyclosporin A, Corn oil, and a stearate surfactant.

Another embodiment comprises cyclosporin A, Arachis oil, and a stearate surfactant.

Another embodiment comprises cyclosporin A, Cottonseed oil, and a stearate surfactant.

Another embodiment comprises cyclosporin A, Safflower oil, and a stearate surfactant.

Another embodiment comprises cyclosporin A, Maize oil, and a stearate surfactant.

Another embodiment comprises cyclosporin A, Linseed oil, and a stearate surfactant.

Another embodiment comprises cyclosporin A, Rapeseed oil, and a stearate surfactant.

Another embodiment comprises cyclosporin A, Soybean oil, and a stearate surfactant.

Another embodiment comprises cyclosporin A, Olive oil, and a stearate surfactant.

Another embodiment comprises cyclosporin A, Caraway oil, and a stearate surfactant.

Another embodiment comprises cyclosporin A, Rosemary oil, and a stearate surfactant.

Another embodiment comprises cyclosporin A, Peanut oil, and a stearate surfactant.

Another embodiment comprises cyclosporin A, Peppermint oil, and a stearate surfactant.

Another embodiment comprises cyclosporin A, Sunflower oil, and a stearate surfactant.

Another embodiment comprises cyclosporin A, Eucalyptus oil, and a stearate surfactant.

Another embodiment comprises cyclosporin A, Sesame oil, and a stearate surfactant.

Another embodiment comprises cyclosporin A, an oil having a specific gravity below 0.9, and a stearate surfactant.

Another embodiment comprises cyclosporin A, Mineral oil, and a stearate surfactant.

Another embodiment comprises cyclosporin A, Coriander oil, and a stearate surfactant.

Another embodiment comprises cyclosporin A, Lavender oil, and a stearate sunfactant.

Another embodiment comprises cyclosporin A, Citronella oil, and a stearate surfactant.

Another embodiment comprises cyclosporin A, Juniper oil, and a stearate surfactant.

Another embodiment comprises cyclosporin A, Lemon oil, and a stearate surfactant.

Another embodiment comprises cyclosporin A, Orange oil, and a stearate surfactant.

Another embodiment comprises cyclosporin A, Clary sage oil, and a stearate surfactant.

Another embodiment comprises cyclosporin A, Nutmeg oil, and a stearate surfactant.

Another embodiment comprises cyclosporin A, Tea tree oil, and a stearate surfactant.

Another embodiment comprises cyclosporin A, an oil having a specific gravity from 0.95 to 1.07, and glyceryl stearate.

Another embodiment comprises cyclosporin A, Anise oil, and glyceryl stearate.

Another embodiment comprises cyclosporin A, Castor oil, and glyceryl stearate.

Another embodiment comprises cyclosporin A, Clove oil, and glyceryl stearate.

Another embodiment comprises cyclosporin A, Cassia oil, and glyceryl stearate.

Another embodiment comprises cyclosporin A, Cinnamon oil, and glyceryl stearate.

Another embodiment comprises cyclosporin A, an oil having a specific gravity from 0.90 to 0.95, and glyceryl stearate.

Another embodiment comprises cyclosporin A, Almond oil, and glyceryl stearate.

Another embodiment comprises cyclosporin A, Corn oil, and glyceryl stearate.

Another embodiment comprises cyclosporin A, Arachis oil, and glyceryl stearate.

Another embodiment comprises cyclosporin A, Cottonseed oil, and glyceryl stearate.

Another embodiment comprises cyclosporin A, Safflower oil, and glyceryl stearate.

Another embodiment comprises cyclosporin A, Maize oil, and glyceryl stearate.

Another embodiment comprises cyclosporin A, Linseed oil, and glyceryl stearate.

Another embodiment comprises cyclosporin A, Rapeseed oil, and glyceryl stearate.

Another embodiment comprises cyclosporin A, Soybean oil, and glyceryl stearate.

Another embodiment comprises cyclosporin A, Olive oil, and glyceryl stearate.

Another embodiment comprises cyclosporin A, Caraway oil, and glyceryl stearate.

Another embodiment comprises cyclosporin A, Rosemary oil, and glyceryl stearate.

Another embodiment comprises cyclosporin A, Peanut oil, and glyceryl stearate.

Another embodiment comprises cyclosporin A, Peppermint oil, and glyceryl stearate.

Another embodiment comprises cyclosporin A, Sunflower oil, and glyceryl stearate.

Another embodiment comprises cyclosporin A, Eucalyptus oil, and glyceryl stearate.

Another embodiment comprises cyclosporin A, Sesame oil, and glyceryl stearate.

Another embodiment comprises cyclosporin A, an oil having a specific gravity below 0.9, and glyceryl stearate.

Another embodiment comprises cyclosporin A, Mineral oil, and glyceryl stearate.

Another embodiment comprises cyclosporin A, Coriander oil, and glyceryl stearate.

Another embodiment comprises cyclosporin A, Lavender oil, and glyceryl stearate.

Another embodiment comprises cyclosporin A, Citronella oil, and glyceryl stearate.

Another embodiment comprises cyclosporin A, Juniper oil, and glyceryl stearate.

Another embodiment comprises cyclosporin A, Lemon oil, and glyceryl stearate.

Another embodiment comprises cyclosporin A, Orange oil, and glyceryl stearate.

Another embodiment comprises cyclosporin A, Clary sage oil, and glyceryl stearate.

Another embodiment comprises cyclosporin A, Nutmeg oil, and glyceryl stearate.

Another embodiment comprises cyclosporin A, Tea tree oil, and glyceryl stearate.

Another embodiment comprises cyclosporin A, an oil having a specific gravity from 0.95 to 1.07, and isopropyl stearate.

Another embodiment comprises cyclosporin A, Anise oil, and isopropyl stearate.

Another embodiment comprises cyclosporin A, Castor oil, and isopropyl stearate.

Another embodiment comprises cyclosporin A, Clove oil, and isopropyl stearate.

Another embodiment comprises cyclosporin A, Cassia oil, and isopropyl stearate.

Another embodiment comprises cyclosporin A, Cinnamon oil, and isopropyl stearate.

Another embodiment comprises cyclosporin A, an oil having a specific gravity from 0.90 to 0.95, and isopropyl stearate.

Another embodiment comprises cyclosporin A, Almond oil, and isopropyl stearate.

Another embodiment comprises cyclosporin A, Corn oil, and isopropyl stearate.

Another embodiment comprises cyclosporin A, Arachis oil, and isopropyl stearate.

Another embodiment comprises cyclosporin A, Cottonseed oil, and isopropyl stearate.

Another embodiment comprises cyclosporin A, Safflower oil, and isopropyl stearate.

Another embodiment comprises cyclosporin A, Maize oil, and isopropyl stearate.

Another embodiment comprises cyclosporin A, Linseed oil, and isopropyl stearate.

Another embodiment comprises cyclosporin A, Rapeseed oil, and isopropyl stearate.

Another embodiment comprises cyclosporin A, Soybean oil, and isopropyl stearate.

Another embodiment comprises cyclosporin A, Olive oil, and isopropyl stearate.

Another embodiment comprises cyclosporin A, Caraway oil, and isopropyl stearate.

Another embodiment comprises cyclosporin A, Rosemary oil, and isopropyl stearate.

Another embodiment comprises cyclosporin A, Peanut oil, and isopropyl stearate.

Another embodiment comprises cyclosporin A, Peppermint oil, and isopropyl stearate.

Another embodiment comprises cyclosporin A, Sunflower oil, and isopropyl stearate.

Another embodiment comprises cyclosporin A, Eucalyptus oil, and isopropyl stearate.

Another embodiment comprises cyclosporin A, Sesame oil, and isopropyl stearate.

Another embodiment comprises cyclosporin A, an oil having a specific gravity below 0.9, and isopropyl stearate.

Another embodiment comprises cyclosporin A, Mineral oil, and isopropyl stearate.

Another embodiment comprises cyclosporin A, Coriander oil, and isopropyl stearate.

Another embodiment comprises cyclosporin A, Lavender oil, and isopropyl stearate.

Another embodiment comprises cyclosporin A, Citronella oil, and isopropyl stearate.

Another embodiment comprises cyclosporin A, Juniper oil, and isopropyl stearate.

Another embodiment comprises cyclosporin A, Lemon oil, and isopropyl stearate.

Another embodiment comprises cyclosporin A, Orange oil, and isopropyl stearate.

Another embodiment comprises cyclosporin A, Clary sage oil, and isopropyl stearate.

Another embodiment comprises cyclosporin A, Nutmeg oil, and isopropyl stearate.

Another embodiment comprises cyclosporin A, Tea tree oil, and isopropyl stearate.

Another embodiment comprises cyclosporin A, an oil having a specific gravity from 0.95 to 1.07, and polyoxyl stearate.

Another embodiment comprises cyclosporin A, Anise oil, and polyoxyl stearate.

Another embodiment comprises cyclosporin A, Castor oil, and polyoxyl stearate.

Another embodiment comprises cyclosporin A, Clove oil, and polyoxyl stearate.

Another embodiment comprises cyclosporin A, Cassia oil, and polyoxyl stearate.

Another embodiment comprises cyclosporin A, Cinnamon oil, and polyoxyl stearate.

Another embodiment comprises cyclosporin A, an oil having a specific gravity from 0.90 to 0.95, and polyoxyl stearate.

Another embodiment comprises cyclosporin A, Almond oil, and polyoxyl stearate.

Another embodiment comprises cyclosporin A, Corn oil, and polyoxyl stearate.

Another embodiment comprises cyclosporin A, Arachis oil, and polyoxyl stearate.

Another embodiment comprises cyclosporin A, Cottonseed oil, and polyoxyl stearate.

Another embodiment comprises cyclosporin A, Safflower oil, and polyoxyl stearate.

Another embodiment comprises cyclosporin A, Maize oil, and polyoxyl stearate.

Another embodiment comprises cyclosporin A, Linseed oil, and polyoxyl stearate.

Another embodiment comprises cyclosporin A, Rapeseed oil, and polyoxyl stearate.

Another embodiment comprises cyclosporin A, Soybean oil, and polyoxyl stearate.

Another embodiment comprises cyclosporin A, Olive oil, and polyoxyl stearate.

Another embodiment comprises cyclosporin A, Caraway oil, and polyoxyl stearate.

Another embodiment comprises cyclosporin A, Rosemary oil, and polyoxyl stearate.

Another embodiment comprises cyclosporin A, Peanut oil, and polyoxyl stearate.

Another embodiment comprises cyclosporin A, Peppermint oil, and polyoxyl stearate.

Another embodiment comprises cyclosporin A, Sunflower oil, and polyoxyl stearate.

Another embodiment comprises cyclosporin A, Eucalyptus oil, and polyoxyl stearate.

Another embodiment comprises cyclosporin A, Sesame oil, and polyoxyl stearate.

Another embodiment comprises cyclosporin A, an oil having a specific gravity below 0.9, and polyoxyl stearate.

Another embodiment comprises cyclosporin A, Mineral oil, and polyoxyl stearate.

Another embodiment comprises cyclosporin A, Coriander oil, and polyoxyl stearate.

Another embodiment comprises cyclosporin A, Lavender oil, and polyoxyl stearate.

Another embodiment comprises cyclosporin A, Citronella oil, and polyoxyl stearate.

Another embodiment comprises cyclosporin A, Juniper oil, and polyoxyl stearate.

Another embodiment comprises cyclosporin A, Lemon oil, and polyoxyl stearate.

Another embodiment comprises cyclosporin A, Orange oil, and polyoxyl stearate.

Another embodiment comprises cyclosporin A, Clary sage oil, and polyoxyl stearate.

Another embodiment comprises cyclosporin A, Nutmeg oil, and polyoxyl stearate.

Another embodiment comprises cyclosporin A, Tea tree oil, and polyoxyl stearate.

Another embodiment comprises cyclosporin A, an oil having a specific gravity from 0.95 to 1.07, and propylene glycol stearate.

Another embodiment comprises cyclosporin A, Anise oil, and propylene glycol stearate.

Another embodiment comprises cyclosporin A, Castor oil, and propylene glycol stearate.

Another embodiment comprises cyclosporin A, Clove oil, and propylene glycol stearate.

Another embodiment comprises cyclosporin A, Cassia oil, and propylene glycol stearate.

Another embodiment comprises cyclosporin A, Cinnamon oil, and propylene glycol stearate.

Another embodiment comprises cyclosporin A, an oil having a specific gravity from 0.90 to 0.95, and propylene glycol stearate.

Another embodiment comprises cyclosporin A, Almond oil, and propylene glycol stearate.

Another embodiment comprises cyclosporin A, Corn oil, and propylene glycol stearate.

Another embodiment comprises cyclosporin A, Arachis oil, and propylene glycol stearate.

Another embodiment comprises cyclosporin A, Cottonseed oil, and propylene glycol stearate.

Another embodiment comprises cyclosporin A, Safflower oil, and propylene glycol stearate.

Another embodiment comprises cyclosporin A, Maize oil, and propylene glycol stearate.

Another embodiment comprises cyclosporin A, Linseed oil, and propylene glycol stearate.

Another embodiment comprises cyclosporin A, Rapeseed oil, and propylene glycol stearate.

Another embodiment comprises cyclosporin A, Soybean oil, and propylene glycol stearate.

Another embodiment comprises cyclosporin A, Olive oil, and propylene glycol stearate.

Another embodiment comprises cyclosporin A, Caraway oil, and propylene glycol stearate.

Another embodiment comprises cyclosporin A, Rosemary oil, and propylene glycol stearate.

Another embodiment comprises cyclosporin A, Peanut oil, and propylene glycol stearate.

Another embodiment comprises cyclosporin A, Peppermint oil, and propylene glycol stearate.

Another embodiment comprises cyclosporin A, Sunflower oil, and propylene glycol stearate.

Another embodiment comprises cyclosporin A, Eucalyptus oil, and propylene glycol stearate.

Another embodiment comprises cyclosporin A, Sesame oil, and propylene glycol stearate.

Another embodiment comprises cyclosporin A, an oil having a specific gravity below 0.9, and propylene glycol stearate.

Another embodiment comprises cyclosporin A, Mineral oil, and propylene glycol stearate.

Another embodiment comprises cyclosporin A, Coriander oil, and propylene glycol stearate.

Another embodiment comprises cyclosporin A, Lavender oil, and propylene glycol stearate.

Another embodiment comprises cyclosporin A, Citronella oil, and propylene glycol stearate.

Another embodiment comprises cyclosporin A, Juniper oil, and propylene glycol stearate.

Another embodiment comprises cyclosporin A, Lemon oil, and propylene glycol stearate.

Another embodiment comprises cyclosporin A, Orange oil, and propylene glycol stearate.

Another embodiment comprises cyclosporin A, Clary sage oil, and propylene glycol stearate.

Another embodiment comprises cyclosporin A, Nutmeg oil, and propylene glycol stearate.

Another embodiment comprises cyclosporin A, Tea tree oil, and propylene glycol stearate.

Another embodiment comprises cyclosporin A, an oil having a specific gravity from 0.95 to 1.07, and sucrose stearate.

Another embodiment comprises cyclosporin A, Anise oil, and sucrose stearate.

Another embodiment comprises cyclosporin A, Castor oil, and sucrose stearate.

Another embodiment comprises cyclosporin A, Clove oil, and sucrose stearate.

Another embodiment comprises cyclosporin A, Cassia oil, and sucrose stearate.

Another embodiment comprises cyclosporin A, Cinnamon oil, and sucrose stearate.

Another embodiment comprises cyclosporin A, an oil having a specific gravity from 0.90 to 0.95, and sucrose stearate.

Another embodiment comprises cyclosporin A, Almond oil, and sucrose stearate.

Another embodiment comprises cyclosporin A, Corn oil, and sucrose stearate.

Another embodiment comprises cyclosporin A, Arachis oil, and sucrose stearate.

Another embodiment comprises cyclosporin A, Cottonseed oil, and sucrose stearate.

Another embodiment comprises cyclosporin A, Safflower oil, and sucrose stearate.

Another embodiment comprises cyclosporin A, Maize oil, and sucrose stearate.

Another embodiment comprises cyclosporin A, Linseed oil, and sucrose stearate.

Another embodiment comprises cyclosporin A, Rapeseed oil, and sucrose stearate.

Another embodiment comprises cyclosporin A, Soybean oil, and sucrose stearate.

Another embodiment comprises cyclosporin A, Olive oil, and sucrose stearate.

Another embodiment comprises cyclosporin A, Caraway oil, and sucrose stearate.

Another embodiment comprises cyclosporin A, Rosemary oil, and sucrose stearate.

Another embodiment comprises cyclosporin A, Peanut oil, and sucrose stearate.

Another embodiment comprises cyclosporin A, Peppermint oil, and sucrose stearate.

Another embodiment comprises cyclosporin A, Sunflower oil, and sucrose stearate.

Another embodiment comprises cyclosporin A, Eucalyptus oil, and sucrose stearate.

Another embodiment comprises cyclosporin A, Sesame oil, and sucrose stearate.

Another embodiment comprises cyclosporin A, an oil having a specific gravity below 0.9, and sucrose stearate.

Another embodiment comprises cyclosporin A, Mineral oil, and sucrose stearate.

Another embodiment comprises cyclosporin A, Coriander oil, and sucrose stearate.

Another embodiment comprises cyclosporin A, Lavender oil, and sucrose stearate.

Another embodiment comprises cyclosporin A, Citronella oil, and sucrose stearate.

Another embodiment comprises cyclosporin A, Juniper oil, and sucrose stearate.

Another embodiment comprises cyclosporin A, Lemon oil, and sucrose stearate.

Another embodiment comprises cyclosporin A, Orange oil, and sucrose stearate.

Another embodiment comprises cyclosporin A, Clary sage oil, and sucrose stearate.

Another embodiment comprises cyclosporin A, Nutmeg oil, and sucrose stearate.

Another embodiment comprises cyclosporin A, Tea tree oil, and sucrose stearate.

Another embodiment comprises cyclosporin A, an oil having a specific gravity from 0.95 to 1.07, and polyethylene glycol.

Another embodiment comprises cyclosporin A, Anise oil, and polyethylene glycol.

Another embodiment comprises cyclosporin A, Castor oil, and polyethylene glycol.

Another embodiment comprises cyclosporin A, Clove oil, and polyethylene glycol.

Another embodiment comprises cyclosporin A, Cassia oil, and polyethylene glycol.

Another embodiment comprises cyclosporin A, Cinnamon oil, and polyethylene glycol.

Another embodiment comprises cyclosporin A, an oil having a specific gravity from 0.90 to 0.95, and polyethylene glycol.

Another embodiment comprises cyclosporin A, Almond oil, and polyethylene glycol.

Another embodiment comprises cyclosporin A, Corn oil, and polyethylene glycol.

Another embodiment comprises cyclosporin A, Arachis oil, and polyethylene glycol.

Another embodiment comprises cyclosporin A, Cottonseed oil, and polyethylene glycol.

Another embodiment comprises cyclosporin A, Safflower oil, and polyethylene glycol.

Another embodiment comprises cyclosporin A, Maize oil, and polyethylene glycol.

Another embodiment comprises cyclosporin A, Linseed oil, and polyethylene glycol.

Another embodiment comprises cyclosporin A, Rapeseed oil, and polyethylene glycol.

Another embodiment comprises cyclosporin A, Soybean oil, and polyethylene glycol.

Another embodiment comprises cyclosporin A, Olive oil, and polyethylene glycol.

Another embodiment comprises cyclosporin A, Caraway oil, and polyethylene glycol.

Another embodiment comprises cyclosporin A, Rosemary oil, and polyethylene glycol.

Another embodiment comprises cyclosporin A, Peanut oil, and polyethylene glycol.

Another embodiment comprises cyclosporin A, Peppermint oil, and polyethylene glycol.

Another embodiment comprises cyclosporin A, Sunflower oil, and polyethylene glycol.

Another embodiment comprises cyclosporin A, Eucalyptus oil, and polyethylene glycol.

Another embodiment comprises cyclosporin A, Sesame oil, and polyethylene glycol.

Another embodiment comprises cyclosporin A, an oil having a specific gravity below 0.9, and polyethylene glycol.

Another embodiment comprises cyclosporin A, Mineral oil, and polyethylene glycol.

Another embodiment comprises cyclosporin A, Coriander oil, and polyethylene glycol.

Another embodiment comprises cyclosporin A, Lavender oil, and polyethylene glycol.

Another embodiment comprises cyclosporin A, Citronella oil, and polyethylene glycol.

Another embodiment comprises cyclosporin A, Juniper oil, and polyethylene glycol.

Another embodiment comprises cyclosporin A, Lemon oil, and polyethylene. glycol.

Another embodiment comprises cyclosporin A, Orange oil, and polyethylene glycol.

Another embodiment comprises cyclosporin A, Clary sage oil, and polyethylene glycol.

Another embodiment comprises cyclosporin A, Nutmeg oil, and polyethylene.glycol.

Another embodiment comprises cyclosporin A, Tea tree oil, and polyethylene glycol.

Another embodiment comprises cyclosporin A, an oil having a specific gravity from 0.95 to 1.07, and polyethylene oxide.

Another embodiment comprises cyclosporin A, Anise oil, and polyethylene oxide.

Another embodiment comprises cyclosporin A, Castor oil, and polyethylene oxide.

Another embodiment comprises cyclosporin A, Clove oil, and polyethylene oxide.

Another embodiment comprises cyclosporin A, Cassia oil, and polyethylene oxide.

Another embodiment comprises cyclosporin A, Cinnamon oil, and polyethylene oxide.

Another embodiment comprises cyclosporin A, an oil having a specific gravity from 0.90 to 0.95, and polyethylene oxide.

Another embodiment comprises cyclosporin A, Almond oil, and polyethylene oxide.

Another embodiment comprises cyclosporin A, Corn oil, and polyethylene oxide.

Another embodiment comprises cyclosporin A, Arachis oil, and polyethylene oxide.

Another embodiment comprises cyclosporin A, Cottonseed oil, and polyethylene oxide.

Another embodiment comprises cyclosporin A, Safflower oil, and polyethylene oxide.

Another embodiment comprises cyclosporin A, Maize oil, and polyethylene oxide.

Another embodiment comprises cyclosporin A, Linseed oil, and polyethylene oxide.

Another embodiment comprises cyclosporin A, Rapeseed oil, and polyethylene oxide.

Another embodiment comprises cyclosporin A, Soybean oil, and polyethylene oxide.

Another embodiment comprises cyclosporin A, Olive oil, and polyethylene oxide.

Another embodiment comprises cyclosporin A, Caraway oil, and polyethylene oxide.

Another embodiment comprises cyclosporin A, Rosemary oil, and polyethylene oxide.

Another embodiment comprises cyclosporin A, Peanut oil, and polyethylene oxide.

Another embodiment comprises cyclosporin A, Peppermint oil, and polyethylene oxide.

Another embodiment comprises cyclosporin A, Sunflower oil, and polyethylene oxide.

Another embodiment comprises cyclosporin A, Eucalyptus oil, and polyethylene oxide.

Another embodiment comprises cyclosporin A, Sesame oil, and polyethylene oxide.

Another embodiment comprises cyclosporin A, an oil having a specific gravity below 0.9, and polyethylene oxide.

Another embodiment comprises cyclosporin A, Mineral oil, and polyethylene oxide.

Another embodiment comprises cyclosporin A, Coriander oil, and polyethylene oxide.

Another embodiment comprises cyclosporin A, Lavender oil, and polyethylene oxide.

Another embodiment comprises cyclosporin A, Citronella oil, and polyethylene oxide.

Another embodiment comprises cyclosporin A, Juniper oil, and polyethylene oxide.

Another embodiment comprises cyclosporin A, Lemon oil, and polyethylene oxide.

Another embodiment comprises cyclosporin A, Orange oil, and polyethylene oxide.

Another embodiment comprises cyclosporin A, Clary sage oil, and polyethylene oxide.

Another embodiment comprises cyclosporin A, Nutmeg oil, and polyethylene oxide.

Another embodiment comprises cyclosporin A, Tea tree oil, and polyethylene oxide.

Another embodiment comprises cyclosporin A, an oil having a specific gravity from 0.95 to 1.07, and polypropylene oxide.

Another embodiment comprises cyclosporin A, Anise oil, and polypropylene oxide.

Another embodiment comprises cyclosporin A, Castor oil, and polypropylene oxide.

Another embodiment comprises cyclosporin A, Clove oil, and polypropylene oxide.

Another embodiment comprises cyclosporin A, Cassia oil, and polypropylene oxide.

Another embodiment comprises cyclosporin A, Cinnamon oil, and polypropylene oxide.

Another embodiment comprises cyclosporin A, an oil having a specific gravity from 0.90 to 0.95, and polypropylene oxide.

Another embodiment comprises cyclosporin A, Almond oil, and polypropylene oxide.

Another embodiment comprises cyclosporin A, Corn oil, and polypropylene oxide.

Another embodiment comprises cyclosporin A, Arachis oil, and polypropylene oxide.

Another embodiment comprises cyclosporin A, Cottonseed oil, and polypropylene oxide.

Another embodiment comprises cyclosporin A, Safflower oil, and polypropylene oxide.

Another embodiment comprises cyclosporin A, Maize oil, and polypropylene oxide.

Another embodiment comprises cyclosporin A, Linseed oil, and polypropylene oxide.

Another embodiment comprises cyclosporin A, Rapeseed oil, and polypropylene oxide.

Another embodiment comprises cyclosporin A, Soybean oil, and polypropylene oxide.

Another embodiment comprises cyclosporin A, Olive oil, and polypropylene oxide.

Another embodiment comprises cyclosporin A, Caraway oil, and polypropylene oxide.

Another embodiment comprises cyclosporin A, Rosemary oil, and polypropylene oxide.

Another embodiment comprises cyclosporin A, Peanut oil, and polypropylene oxide.

Another embodiment comprises cyclosporin A, Peppermint oil, and polypropylene oxide.

Another embodiment comprises cyclosporin A, Sunflower oil, and polypropylene oxide.

Another embodiment comprises cyclosporin A, Eucalyptus oil, and polypropylene oxide.

Another embodiment comprises cyclosporin A, Sesame oil, and polypropylene oxide.

Another embodiment comprises cyclosporin A, an oil having a specific gravity below 0.9, and polypropylene oxide.

Another embodiment comprises cyclosporin A, Mineral oil, and polypropylene oxide.

Another embodiment comprises cyclosporin A, Coriander oil, and polypropylene oxide.

Another embodiment comprises cyclosporin A, Lavender oil, and polypropylene oxide.

Another embodiment comprises cyclosporin A, Citronella oil, and polypropylene oxide.

Another embodiment comprises cyclosporin A, Juniper oil, and polypropylene oxide.

Another embodiment comprises cyclosporin A, Lemon oil, and polypropylene oxide.

Another embodiment comprises cyclosporin A, Orange oil, and polypropylene oxide.

Another embodiment comprises cyclosporin A, Clary sage oil, and polypropylene oxide.

Another embodiment comprises cyclosporin A, Nutmeg oil, and polypropylene oxide.

Another embodiment comprises cyclosporin A, Tea tree oil, and polypropylene oxide.

Another embodiment comprises cyclosporin A, an oil having a specific gravity from 0.95 to 1.07, and a polyethylene oxide-polypropylene oxide copolymer.

Another embodiment comprises cyclosporin A, Anise oil, and a polyethylene oxide-polypropylene oxide copolymer.

Another embodiment comprises cyclosporin A, Castor oil, and a polyethylene oxide-polypropylene oxide copolymer.

Another embodiment comprises cyclosporin A, Clove oil, and a polyethylene oxide-polypropylene oxide copolymer.

Another embodiment comprises cyclosporin A, Cassia oil, and a polyethylene oxide-polypropylene oxide copolymer.

Another embodiment comprises cyclosporin A, Cinnamon oil, and a polyethylene oxide-polypropylene oxide copolymer.

Another embodiment comprises cyclosporin A, an oil having a specific gravity from 0.90 to 0.95, and a polyethylene oxide-polypropylene oxide copolymer.

Another embodiment comprises cyclosporin A, Almond oil, and a polyethylene oxide-polypropylene oxide copolymer.

Another embodiment comprises cyclosporin A, Corn oil, and a polyethylene oxide-polypropylene oxide copolymer.

Another embodiment comprises cyclosporin A, Arachis oil, and a polyethylene oxide-polypropylene oxide copolymer.

Another embodiment comprises cyclosporin A, Cottonseed oil, and a polyethylene oxide-polypropylene oxide copolymer.

Another embodiment comprises cyclosporin A, Safflower oil, and a polyethylene oxide-polypropylene oxide copolymer.

Another embodiment comprises cyclosporin A, Maize oil, and a polyethylene oxide-polypropylene oxide copolymer.

Another embodiment comprises cyclosporin A, Linseed oil, and a polyethylene oxide-polypropylene oxide copolymer.

Another embodiment comprises cyclosporin A, Rapeseed oil, and a polyethylene oxide-polypropylene oxide copolymer.

Another embodiment comprises cyclosporin A, Soybean oil, and a polyethylene oxide-polypropylene oxide copolymer.

Another embodiment comprises cyclosporin A, Olive oil, and a polyethylene oxide-polypropylene oxide copolymer.

Another embodiment comprises cyclosporin A, Caraway oil, and a polyethylene oxide-polypropylene oxide copolymer.

Another embodiment comprises cyclosporin A, Rosemary oil, and a polyethylene oxide-polypropylene oxide copolymer.

Another embodiment comprises cyclosporin A, Peanut oil, and a polyethylene oxide-polypropylene oxide copolymer.

Another embodiment comprises cyclosporin A, Peppermint oil, and a polyethylene oxide-polypropylene oxide copolymer.

Another embodiment comprises cyclosporin A, Sunflower oil, and a polyethylene oxide-polypropylene oxide copolymer.

Another embodiment comprises cyclosporin A, Eucalyptus oil, and a polyethylene oxide-polypropylene oxide copolymer.

Another embodiment comprises cyclosporin A, Sesame oil, and a polyethylene oxide-polypropylene oxide copolymer.

Another embodiment comprises cyclosporin A, an oil having a specific gravity below 0.9, and a polyethylene oxide-polypropylene oxide copolymer.

Another embodiment comprises cyclosporin A, Mineral oil, and a polyethylene oxide-polypropylene oxide copolymer.

Another embodiment comprises cyclosporin A, Coriander oil, and a polyethylene oxide-polypropylene oxide copolymer.

Another embodiment comprises cyclosporin A, Lavender oil, and a polyethylene oxide-polypropylene oxide copolymer.

Another embodiment comprises cyclosporin A, Citronella oil, and a polyethylene oxide-polypropylene oxide copolymer.

Another embodiment comprises cyclosporin A, Juniper oil, and a polyethylene oxide-polypropylene oxide copolymer.

Another embodiment comprises cyclosporin A, Lemon oil, and a polyethylene oxide-polypropylene oxide copolymer.

Another embodiment comprises cyclosporin A, Orange oil, and a polyethylene oxide-polypropylene oxide copolymer.

Another embodiment comprises cyclosporin A, Clary sage oil, and a polyethylene oxide-polypropylene oxide copolymer.

Another embodiment comprises cyclosporin A, Nutmeg oil, and a polyethylene oxide-polypropylene oxide copolymer.

Another embodiment comprises cyclosporin A, Tea tree oil, and a polyethylene oxide-polypropylene oxide copolymer.

Another embodiment comprises cyclosporin A, an oil having a specific gravity from 0.95 to 1.07, and an alcohol ethoxylate.

Another embodiment comprises cyclosporin A, Anise oil, and an alcohol ethoxylate.

Another embodiment comprises cyclosporin A, Castor oil, and an alcohol ethoxylate.

Another embodiment comprises cyclosporin A, Clove oil, and an alcohol ethoxylate.

Another embodiment comprises cyclosporin A, Cassia oil, and an alcohol ethoxylate.

Another embodiment comprises cyclosporin A, Cinnamon oil, and an alcohol ethoxylate.

Another embodiment comprises cyclosporin A, an oil having a specific gravity from 0.90 to 0.95, and an alcohol ethoxylate.

Another embodiment comprises cyclosporin A, Almond oil, and an alcohol ethoxylate.

Another embodiment comprises cyclosporin A, Corn oil, and an alcohol ethoxylate.

Another embodiment comprises cyclosporin A, Arachis oil, and an alcohol ethoxylate.

Another embodiment comprises cyclosporin A, Cottonseed oil, and an alcohol ethoxylate.

Another embodiment comprises cyclosporin A, Safflower oil, and an alcohol ethoxylate.

Another embodiment comprises cyclosporin A, Maize oil, and an alcohol ethoxylate.

Another embodiment comprises cyclosporin A, Linseed oil, and an alcohol ethoxylate.

Another embodiment comprises cyclosporin A, Rapeseed oil, and an alcohol ethoxylate.

Another embodiment comprises cyclosporin A, Soybean oil, and an alcohol ethoxylate.

Another embodiment comprises cyclosporin A, Olive oil, and an alcohol ethoxylate.

Another embodiment comprises cyclosporin A, Caraway oil, and an alcohol ethoxylate.

Another embodiment comprises cyclosporin A, Rosemary oil, and an alcohol ethoxylate.

Another embodiment comprises cyclosporin A, Peanut oil, and an alcohol ethoxylate.

Another embodiment comprises cyclosporin A, Peppermint oil, and an alcohol ethoxylate.

Another embodiment comprises cyclosporin A, Sunflower oil, and an alcohol ethoxylate.

Another embodiment comprises cyclosporin A, Eucalyptus oil, and an alcohol ethoxylate.

Another embodiment comprises cyclosporin A, Sesame oil, and an alcohol ethoxylate.

Another embodiment comprises cyclosporin A, an oil having a specific gravity below 0.9, and an alcohol ethoxylate.

Another embodiment comprises cyclosporin A, Mineral oil, and an alcohol ethoxylate.

Another embodiment comprises cyclosporin A, Coriander oil, and an alcohol ethoxylate.

Another embodiment comprises cyclosporin A, Lavender oil, and an alcohol ethoxylate.

Another embodiment comprises cyclosporin A, Citronella oil, and an alcohol ethoxylate.

Another embodiment comprises cyclosporin A, Juniper oil, and an alcohol ethoxylate.

Another embodiment comprises cyclosporin A, Lemon oil, and an alcohol ethoxylate.

Another embodiment comprises cyclosporin A, Orange oil, and an alcohol ethoxylate.

Another embodiment comprises cyclosporin A, Clary sage oil, and an alcohol ethoxylate.

Another embodiment comprises cyclosporin A, Nutmeg oil, and an alcohol ethoxylate.

Another embodiment comprises cyclosporin A, Tea tree oil, and an alcohol ethoxylate.

Another embodiment comprises cyclosporin A, an oil having a specific gravity from 0.95 to 1.07, and an alkylphenol ethoxylate.

Another embodiment comprises cyclosporin A, Anise oil, and an alkylphenol ethoxylate.

Another embodiment comprises cyclosporin A, Castor oil, and an alkylphenol ethoxylate.

Another embodiment comprises cyclosporin A, Clove oil, and an alkylphenol ethoxylate.

Another embodiment comprises cyclosporin A, Cassia oil, and an alkylphenol ethoxylate.

Another embodiment comprises cyclosporin A, Cinnamon oil, and an alkylphenol ethoxylate.

Another embodiment comprises cyclosporin A, an oil having a specific gravity from 0.90 to 0.95, and an alkylphenol ethoxylate.

Another embodiment comprises cyclosporin A, Almond oil, and an alkylphenol ethoxylate.

Another embodiment comprises cyclosporin A, Corn oil, and an alkylphenol ethoxylate.

Another embodiment comprises cyclosporin A, Arachis oil, and an alkylphenol ethoxylate.

Another embodiment comprises cyclosporin A, Cottonseed oil, and an alkylphenol ethoxylate.

Another embodiment comprises cyclosporin A, Safflower oil, and an alkylphenol ethoxylate.

Another embodiment comprises cyclosporin A, Maize oil, and an alkylphenol ethoxylate.

Another embodiment comprises cyclosporin A, Linseed oil, and an alkylphenol ethoxylate.

Another embodiment comprises cyclosporin A, Rapeseed oil, and an alkylphenol ethoxylate.

Another embodiment comprises cyclosporin A, Soybean oil, and an alkylphenol ethoxylate.

Another embodiment comprises cyclosporin A, Olive oil, and an alkylphenol ethoxylate.

Another embodiment comprises cyclosporin A, Caraway oil, and an alkylphenol ethoxylate.

Another embodiment comprises cyclosporin A, Rosemary oil, and an alkylphenol ethoxylate.

Another embodiment comprises cyclosporin A, Peanut oil, and an alkylphenol ethoxylate.

Another embodiment comprises cyclosporin A, Peppermint oil, and an alkylphenol ethoxylate.

Another embodiment comprises cyclosporin A, Sunflower oil, and an alkylphenol ethoxylate.

Another embodiment comprises cyclosporin A, Eucalyptus oil, and an alkylphenol ethoxylate.

Another embodiment comprises cyclosporin A, Sesame oil, and an alkylphenol ethoxylate.

Another embodiment comprises cyclosporin A, an oil having a specific gravity below 0.9, and an alkylphenol ethoxylate.

Another embodiment comprises cyclosporin A, Mineral oil, and an alkylphenol ethoxylate.

Another embodiment comprises cyclosporin A, Coriander oil, and an alkylphenol ethoxylate.

Another embodiment comprises cyclosporin A, Lavender oil, and an alkylphenol ethoxylate.

Another embodiment comprises cyclosporin A, Citronella oil, and an alkylphenol ethoxylate.

Another embodiment comprises cyclosporin A, Juniper oil, and an alkylphenol ethoxylate.

Another embodiment comprises cyclosporin A, Lemon oil, and an alkylphenol ethoxylate.

Another embodiment comprises cyclosporin A, Orange oil, and an alkylphenol ethoxylate.

Another embodiment comprises cyclosporin A, Clary sage oil, and an alkylphenol ethoxylate.

Another embodiment comprises cyclosporin A, Nutmeg oil, and an alkylphenol ethoxylate.

Another embodiment comprises cyclosporin A, Tea tree oil, and an alkylphenol ethoxylate.

Another embodiment comprises cyclosporin A, an oil having a specific gravity from 0.95 to 1.07, and an alkyl glycoside.

Another embodiment comprises cyclosporin A, Anise oil, and an alkyl glycoside.

Another embodiment comprises cyclosporin A, Castor oil, and an alkyl glycoside.

Another embodiment comprises cyclosporin A, Clove oil, and an alkyl glycoside.

Another embodiment comprises cyclosporin A, Cassia oil, and an alkyl glycoside.

Another embodiment comprises cyclosporin A, Cinnamon oil, and an alkyl glycoside.

Another embodiment comprises cyclosporin A, an oil having a specific gravity from 0.90 to 0.95, and an alkyl glycoside.

Another embodiment comprises cyclosporin A, Almond oil, and an alkyl glycoside.

Another embodiment comprises cyclosporin A, Corn oil, and an alkyl glycoside.

Another embodiment comprises cyclosporin A, Arachis oil, and an alkyl glycoside.

Another embodiment comprises cyclosporin A, Cottonseed oil, and an alkyl glycoside.

Another embodiment comprises cyclosporin A, Safflower oil, and an alkyl glycoside.

Another embodiment comprises cyclosporin A, Maize oil, and an alkyl glycoside.

Another embodiment comprises cyclosporin A, Linseed oil, and an alkyl glycoside.

Another embodiment comprises cyclosporin A, Rapeseed oil, and an alkyl glycoside.

Another embodiment comprises cyclosporin A, Soybean oil, and an alkyl glycoside.

Another embodiment comprises cyclosporin A, Olive oil, and an alkyl glycoside.

Another embodiment comprises cyclosporin A, Caraway oil, and an alkyl glycoside.

Another embodiment comprises cyclosporin A, Rosemary oil, and an alkyl glycoside.

Another embodiment comprises cyclosporin A, Peanut oil, and an alkyl glycoside.

Another embodiment comprises cyclosporin A, Peppermint oil, and an alkyl glycoside.

Another embodiment comprises cyclosporin A, Sunflower oil, and an alkyl glycoside.

Another embodiment comprises cyclosporin A, Eucalyptus oil, and an alkyl glycoside.

Another embodiment comprises cyclosporin A, Sesame oil, and an alkyl glycoside.

Another embodiment comprises cyclosporin A, an oil having a specific gravity below 0.9, and an alkyl glycoside.

Another embodiment comprises cyclosporin A, Mineral oil, and an alkyl glycoside.

Another embodiment comprises cyclosporin A, Coriander oil, and an alkyl glycoside.

Another embodiment comprises cyclosporin A, Lavender oil, and an alkyl glycoside.

Another embodiment comprises cyclosporin A, Citronella oil, and an alkyl glycoside.

Another embodiment comprises cyclosporin A, Juniper oil, and an alkyl glycoside.

Another embodiment comprises cyclosporin A, Lemon oil, and an alkyl glycoside.

Another embodiment comprises cyclosporin A, Orange oil, and an alkyl glycoside.

Another embodiment comprises cyclosporin A, Clary sage oil, and an alkyl glycoside.

Another embodiment comprises cyclosporin A, Nutmeg oil, and an alkyl glycoside.

Another embodiment comprises cyclosporin A, Tea tree oil, and an alkyl glycoside.

Another embodiment comprises cyclosporin A, an oil having a specific gravity from 0.95 to 1.07, and an alkyl polyglycoside.

Another embodiment comprises cyclosporin A, Anise oil, and an alkyl polyglycoside.

Another embodiment comprises cyclosporin A, Castor oil, and an alkyl polyglycoside.

Another embodiment comprises cyclosporin A, Clove oil, and an alkyl polyglycoside.

Another embodiment comprises cyclosporin A, Cassia oil, and an alkyl polyglycoside.

Another embodiment comprises cyclosporin A, Cinnamon oil, and an alkyl polyglycoside.

Another embodiment comprises cyclosporin A, an oil having a specific gravity from 0.90 to 0.95, and an alkyl polyglycoside.

Another embodiment comprises cyclosporin A, Almond oil, and an alkyl polyglycoside.

Another embodiment comprises cyclosporin A, Corn oil, and an alkyl polyglycoside.

Another embodiment comprises cyclosporin A, Arachis oil, and an alkyl polyglycoside.

Another embodiment comprises cyclosporin A, Cottonseed oil, and an alkyl polyglycoside.

Another embodiment comprises cyclosporin A, Safflower oil, and an alkyl polyglycoside.

Another embodiment comprises cyclosporin A, Maize oil, and an alkyl polyglycoside.

Another embodiment comprises cyclosporin A, Linseed oil, and an alkyl polyglycoside.

Another embodiment comprises cyclosporin A, Rapeseed oil, and an alkyl polyglycoside.

Another embodiment comprises cyclosporin A, Soybean oil, and an alkyl polyglycoside.

Another embodiment comprises cyclosporin A, Olive oil, and an alkyl polyglycoside.

Another embodiment comprises cyclosporin A, Caraway oil, and an alkyl polyglycoside.

Another embodiment comprises cyclosporin A, Rosemary oil, and an alkyl polyglycoside.

Another embodiment comprises cyclosporin A, Peanut oil, and an alkyl polyglycoside.

Another embodiment comprises cyclosporin A, Peppermint oil, and an alkyl polyglycoside.

Another embodiment comprises cyclosporin A, Sunflower oil, and an alkyl polyglycoside.

Another embodiment comprises cyclosporin A, Eucalyptus oil, and an alkyl polyglycoside.

Another embodiment comprises cyclosporin A, Sesame oil, and an alkyl polyglycoside.

Another embodiment comprises cyclosporin A, an oil having a specific gravity below 0.9, and an alkyl polyglycoside.

Another embodiment comprises cyclosporin A, Mineral oil, and an alkyl polyglycoside.

Another embodiment comprises cyclosporin A, Coriander oil, and an alkyl polyglycoside.

Another embodiment comprises cyclosporin A, Lavender oil, and an alkyl polyglycoside.

Another embodiment comprises cyclosporin A, Citronella oil, and an alkyl polyglycoside.

Another embodiment comprises cyclosporin A, Juniper oil, and an alkyl polyglycoside.

Another embodiment comprises cyclosporin A, Lemon oil, and an alkyl polyglycoside.

Another embodiment comprises cyclosporin A, Orange oil, and an alkyl polyglycoside.

Another embodiment comprises cyclosporin A, Clary sage oil, and an alkyl polyglycoside.

Another embodiment comprises cyclosporin A, Nutmeg oil, and an alkyl polyglycoside.

Another embodiment comprises cyclosporin A, Tea tree oil, and an alkyl polyglycoside.

Another embodiment comprises cyclosporin A, an oil having a specific gravity from 0.95 to 1.07, and a fatty alcohol.

Another embodiment comprises cyclosporin A, Anise oil, and a fatty alcohol.

Another embodiment comprises cyclosporin A, Castor oil, and a fatty alcohol.

Another embodiment comprises cyclosporin A, Clove oil, and a fatty alcohol.

Another embodiment comprises cyclosporin A, Cassia oil, and a fatty alcohol.

Another embodiment comprises cyclosporin A, Cinnamon oil, and a fatty alcohol.

Another embodiment comprises cyclosporin A, an oil having a specific gravity from 0.90 to 0.95, and a fatty alcohol.

Another embodiment comprises cyclosporin A, Almond oil, and a fatty alcohol.

Another embodiment comprises cyclosporin A, Corn oil, and a fatty alcohol.

Another embodiment comprises cyclosporin A, Arachis oil, and a fatty alcohol.

Another embodiment comprises cyclosporin A, Cottonseed oil, and a fatty alcohol.

Another embodiment comprises cyclosporin A, Safflower oil, and a fatty alcohol.

Another embodiment comprises cyclosporin A, Maize oil, and a fatty alcohol.

Another embodiment comprises cyclosporin A, Linseed oil, and a fatty alcohol.

Another embodiment comprises cyclosporin A, Rapeseed oil, and a fatty alcohol.

Another embodiment comprises cyclosporin A, Soybean oil, and a fatty alcohol.

Another embodiment comprises cyclosporin A, Olive oil, and a fatty alcohol.

Another embodiment comprises cyclosporin A, Caraway oil, and a fatty alcohol.

Another embodiment comprises cyclosporin A, Rosemary oil, and a fatty alcohol.

Another embodiment comprises cyclosporin A, Peanut oil, and a fatty alcohol.

Another embodiment comprises cyclosporin A, Peppermint oil, and a fatty alcohol.

Another embodiment comprises cyclosporin A, Sunflower oil, and a fatty alcohol.

Another embodiment comprises cyclosporin A, Eucalyptus oil, and a fatty alcohol.

Another embodiment comprises cyclosporin A, Sesame oil, and a fatty alcohol.

Another embodiment comprises cyclosporin A, an oil having a specific gravity below 0.9, and a fatty alcohol.

Another embodiment comprises cyclosporin A, Mineral oil, and a fatty alcohol.

Another embodiment comprises cyclosporin A, Coriander oil, and a fatty alcohol.

Another embodiment comprises cyclosporin A, Lavender oil, and a fatty alcohol.

Another embodiment comprises cyclosporin A, Citronella oil, and a fatty alcohol.

Another embodiment comprises cyclosporin A, Juniper oil, and a fatty alcohol.

Another embodiment comprises cyclosporin A, Lemon oil, and a fatty alcohol.

Another embodiment comprises cyclosporin A, Orange oil, and a fatty alcohol.

Another embodiment comprises cyclosporin A, Clary sage oil, and a fatty alcohol.

Another embodiment comprises cyclosporin A, Nutmeg oil, and a fatty alcohol.

Another embodiment comprises cyclosporin A, Tea tree oil, and a fatty alcohol.

Another embodiment comprises cyclosporin A, an oil having a specific gravity from 0.95 to 1.07, and a cellulose derivative.

Another embodiment comprises cyclosporin A, Anise oil, and a cellulose derivative.

Another embodiment comprises cyclosporin A, Castor oil, and a cellulose derivative.

Another embodiment comprises cyclosporin A, Clove oil, and a cellulose derivative.

Another embodiment comprises cyclosporin A, Cassia oil, and a cellulose derivative.

Another embodiment comprises cyclosporin A, Cinnamon oil, and a cellulose derivative.

Another embodiment comprises cyclosporin A, an oil having a specific gravity from 0.90 to 0.95, and a cellulose derivative.

Another embodiment comprises cyclosporin A, Almond oil, and a cellulose derivative.

Another embodiment comprises cyclosporin A, Corn oil, and a cellulose derivative.

Another embodiment comprises cyclosporin A, Arachis oil, and a cellulose derivative.

Another embodiment comprises cyclosporin A, Cottonseed oil, and a cellulose derivative.

Another embodiment comprises cyclosporin A, Safflower oil, and a cellulose derivative.

Another embodiment comprises cyclosporin A, Maize oil, and a cellulose derivative.

Another embodiment comprises cyclosporin A, Linseed oil, and a cellulose derivative.

Another embodiment comprises cyclosporin A, Rapeseed oil, and a cellulose derivative.

Another embodiment comprises cyclosporin A, Soybean oil, and a cellulose derivative.

Another embodiment comprises cyclosporin A, Olive oil, and a cellulose derivative.

Another embodiment comprises cyclosporin A, Caraway oil, and a cellulose derivative.

Another embodiment comprises cyclosporin A, Rosemary oil, and a cellulose derivative.

Another embodiment comprises cyclosporin A, Peanut oil, and a cellulose derivative.

Another embodiment comprises cyclosporin A, Peppermint oil, and a cellulose derivative.

Another embodiment comprises cyclosporin A, Sunflower oil, and a cellulose derivative.

Another embodiment comprises cyclosporin A, Eucalyptus oil, and a cellulose derivative.

Another embodiment comprises cyclosporin A, Sesame oil, and a cellulose derivative.

Another embodiment comprises cyclosporin A, an oil having a specific gravity below 0.9, and a cellulose derivative.

Another embodiment comprises cyclosporin A, Mineral oil, and a cellulose derivative.

Another embodiment comprises cyclosporin A, Coriander oil, and a cellulose derivative.

Another embodiment comprises cyclosporin A, Lavender oil, and a cellulose derivative.

Another embodiment comprises cyclosporin A, Citronella oil, and a cellulose derivative.

Another embodiment comprises cyclosporin A, Juniper oil, and a cellulose derivative.

Another embodiment comprises cyclosporin A, Lemon oil, and a cellulose derivative.

Another embodiment comprises cyclosporin A, Orange oil, and a cellulose derivative.

Another embodiment comprises cyclosporin A, Clary sage oil, and a cellulose derivative.

Another embodiment comprises cyclosporin A, Nutmeg oil, and a cellulose derivative.

Another embodiment comprises cyclosporin A, Tea tree oil, and a cellulose derivative.

Another embodiment comprises cyclosporin A, an oil having a specific gravity from 0.95 to 1.07, and hydroxypropylmethyl cellulose.

Another embodiment comprises cyclosporin A, Anise oil, and hydroxypropylmethyl cellulose.

Another embodiment comprises cyclosporin A, Castor oil, and hydroxypropylmethyl cellulose.

Another embodiment comprises cyclosporin A, Clove oil, and hydroxypropylmethyl cellulose.

Another embodiment comprises cyclosporin A, Cassia oil, and hydroxypropylmethyl cellulose.

Another embodiment comprises cyclosporin A, Cinnamon oil, and hydroxypropylmethyl cellulose.

Another embodiment comprises cyclosporin A, an oil having a specific gravity from 0.90 to 0.95, and hydroxypropylmethyl cellulose.

Another embodiment comprises cyclosporin A, Almond oil, and hydroxypropylmethyl cellulose.

Another embodiment comprises cyclosporin A, Corn oil, and hydroxypropylmethyl cellulose.

Another embodiment comprises cyclosporin A, Arachis oil, and hydroxypropylmethyl cellulose.

Another embodiment comprises cyclosporin A, Cottonseed oil, and hydroxypropylmethyl cellulose.

Another embodiment comprises cyclosporin A, Safflower oil, and hydroxypropylmethyl cellulose.

Another embodiment comprises cyclosporin A, Maize oil, and hydroxypropylmethyl cellulose.

Another embodiment comprises cyclosporin A, Linseed oil, and hydroxypropylmethyl cellulose.

Another embodiment comprises cyclosporin A, Rapeseed oil, and hydroxypropylmethyl cellulose.

Another embodiment comprises cyclosporin A, Soybean oil, and hydroxypropylmethyl cellulose.

Another embodiment comprises cyclosporin A, Olive oil, and hydroxypropylmethyl cellulose.

Another embodiment comprises cyclosporin A, Caraway oil, and hydroxypropylmethyl cellulose.

Another embodiment comprises cyclosporin A, Rosemary oil, and hydroxypropylmethyl cellulose.

Another embodiment comprises cyclosporin A, Peanut oil, and hydroxypropylmethyl cellulose.

Another embodiment comprises cyclosporin A, Peppermint oil, and hydroxypropylmethyl cellulose.

Another embodiment comprises cyclosporin A, Sunflower oil, and hydroxypropylmethyl cellulose.

Another embodiment comprises cyclosporin A, Eucalyptus oil, and hydroxypropylmethyl cellulose.

Another embodiment comprises cyclosporin A, Sesame oil, and hydroxypropylmethyl cellulose.

Another embodiment comprises cyclosporin A, an oil having a specific gravity below 0.9, and hydroxypropylmethyl cellulose.

Another embodiment comprises cyclosporin A, Mineral oil, and hydroxypropylmethyl cellulose.

Another embodiment comprises cyclosporin A, Coriander oil, and hydroxypropylmethyl cellulose.

Another embodiment comprises cyclosporin A, Lavender oil, and hydroxypropylmethyl cellulose.

Another embodiment comprises cyclosporin A, Citronella oil, and hydroxypropylmethyl cellulose.

Another embodiment comprises cyclosporin A, Juniper oil, and hydroxypropylmethyl cellulose.

Another embodiment comprises cyclosporin A, Lemon oil, and hydroxypropylmethyl cellulose.

Another embodiment comprises cyclosporin A, Orange oil, and hydroxypropylmethyl cellulose.

Another embodiment comprises cyclosporin A, Clary sage oil, and hydroxypropylmethyl cellulose.

Another embodiment comprises cyclosporin A, Nutmeg oil, and hydroxypropylmethyl cellulose.

Another embodiment comprises cyclosporin A, Tea tree oil, and hydroxypropylmethyl cellulose.

Another embodiment comprises cyclosporin A, an oil having a specific gravity from 0.95 to 1.07, and carboxymethyl cellulose.

Another embodiment comprises cyclosporin A, Anise oil, and carboxymethyl cellulose.

Another embodiment comprises cyclosporin A, Castor oil, and carboxymethyl cellulose.

Another embodiment comprises cyclosporin A, Clove oil, and carboxymethyl cellulose.

Another embodiment comprises cyclosporin A, Cassia oil, and carboxymethyl cellulose.

Another embodiment comprises cyclosporin A, Cinnamon oil, and carboxymethyl cellulose.

Another embodiment comprises cyclosporin A, an oil having a specific gravity from 0.90 to 0.95, and carboxymethyl cellulose.

Another embodiment comprises cyclosporin A, Almond oil, and carboxymethyl cellulose.

Another embodiment comprises cyclosporin A, Corn oil, and carboxymethyl cellulose.

Another embodiment comprises cyclosporin A, Arachis oil, and carboxymethyl cellulose.

Another embodiment comprises cyclosporin A, Cottonseed oil, and carboxymethyl cellulose.

Another embodiment comprises cyclosporin A, Safflower oil, and carboxymethyl cellulose.

Another embodiment comprises cyclosporin A, Maize oil, and carboxymethyl cellulose.

Another embodiment comprises cyclosporin A, Linseed oil, and carboxymethyl cellulose.

Another embodiment comprises cyclosporin A, Rapeseed oil, and carboxymethyl cellulose.

Another embodiment comprises cyclosporin A, Soybean oil, and carboxymethyl cellulose.

Another embodiment comprises cyclosporin A, Olive oil, and carboxymethyl cellulose.

Another embodiment comprises cyclosporin A, Caraway oil, and carboxymethyl cellulose.

Another embodiment comprises cyclosporin A, Rosemary oil, and carboxymethyl cellulose.

Another embodiment comprises cyclosporin A, Peanut oil, and carboxymethyl cellulose.

Another embodiment comprises cyclosporin A, Peppermint oil, and carboxymethyl cellulose.

Another embodiment comprises cyclosporin A, Sunflower oil, and carboxymethyl cellulose.

Another embodiment comprises cyclosporin A, Eucalyptus oil, and carboxymethyl cellulose.

Another embodiment comprises cyclosporin A, Sesame oil, and carboxymethyl cellulose.

Another embodiment comprises cyclosporin A, an oil having a specific gravity below 0.9, and carboxymethyl cellulose.

Another embodiment comprises cyclosporin A, Mineral oil, and carboxymethyl cellulose.

Another embodiment comprises cyclosporin A, Coriander oil, and carboxymethyl cellulose.

Another embodiment comprises cyclosporin A, Lavender oil, and carboxymethyl cellulose.

Another embodiment comprises cyclosporin A, Citronella oil, and carboxymethyl cellulose.

Another embodiment comprises cyclosporin A, Juniper oil, and carboxymethyl cellulose.

Another embodiment comprises cyclosporin A, Lemon oil, and carboxymethyl cellulose.

Another embodiment comprises cyclosporin A, Orange oil, and carboxymethyl cellulose.

Another embodiment comprises cyclosporin A, Clary sage oil, and carboxymethyl cellulose.

Another embodiment comprises cyclosporin A, Nutmeg oil, and carboxymethyl cellulose.

Another embodiment comprises cyclosporin A, Tea tree oil, and carboxymethyl cellulose.

Another embodiment comprises cyclosporin A, an oil having a specific gravity from 0.95 to 1.07, and a polyacrylic acid.

Another embodiment comprises cyclosporin A, Anise oil, and a polyacrylic acid.

Another embodiment comprises cyclosporin A, Castor oil, and a polyacrylic acid.

Another embodiment comprises cyclosporin A, Clove oil, and a polyacrylic acid.

Another embodiment comprises cyclosporin A, Cassia oil, and a polyacrylic acid.

Another embodiment comprises cyclosporin A, Cinnamon oil, and a polyacrylic acid.

Another embodiment comprises cyclosporin A, an oil having a specific gravity from 0.90 to 0.95, and a polyacrylic acid.

Another embodiment comprises cyclosporin A, Almond oil, and a polyacrylic acid.

Another embodiment comprises cyclosporin A, Corn oil, and a polyacrylic acid.

Another embodiment comprises cyclosporin A, Arachis oil, and a polyacrylic acid.

Another embodiment comprises cyclosporin A, Cottonseed oil, and a polyacrylic acid.

Another embodiment comprises cyclosporin A, Safflower oil, and a polyacrylic acid.

Another embodiment comprises cyclosporin A, Maize oil, and a polyacrylic acid.

Another embodiment comprises cyclosporin A, Linseed oil, and a polyacrylic acid.

Another embodiment comprises cyclosporin A, Rapeseed oil, and a polyacrylic acid.

Another embodiment comprises cyclosporin A, Soybean oil, and a polyacrylic acid.

Another embodiment comprises cyclosporin A, Olive oil, and a polyacrylic acid.

Another embodiment comprises cyclosporin A, Caraway oil, and a polyacrylic acid.

Another embodiment comprises cyclosporin A, Rosemary oil, and a polyacrylic acid.

Another embodiment comprises cyclosporin A, Peanut oil, and a polyacrylic acid.

Another embodiment comprises cyclosporin A, Peppermint oil, and a polyacrylic acid.

Another embodiment comprises cyclosporin A, Sunflower oil, and a polyacrylic acid.

Another embodiment comprises cyclosporin A, Eucalyptus oil, and a polyacrylic acid.

Another embodiment comprises cyclosporin A, Sesame oil, and a polyacrylic acid.

Another embodiment comprises cyclosporin A, an oil having a specific gravity below 0.9, and a polyacrylic acid.

Another embodiment comprises cyclosporin A, Mineral oil, and a polyacrylic acid.

Another embodiment comprises cyclosporin A, Coriander oil, and a polyacrylic acid.

Another embodiment comprises cyclosporin A, Lavender oil, and a polyacrylic acid.

Another embodiment comprises cyclosporin A, Citronella oil, and a polyacrylic acid.

Another embodiment comprises cyclosporin A, Juniper oil, and a polyacrylic acid.

Another embodiment comprises cyclosporin A, Lemon oil, and a polyacrylic acid.

Another embodiment comprises cyclosporin A, Orange oil, and a polyacrylic acid.

Another embodiment comprises cyclosporin A, Clary sage oil, and a polyacrylic acid.

Another embodiment comprises cyclosporin A, Nutmeg oil, and a polyacrylic acid.

Another embodiment comprises cyclosporin A, Tea tree oil, and a polyacrylic acid.

Another embodiment comprises cyclosporin A, an oil having a specific gravity from 0.95 to 1.07, and a Carbomer.

Another embodiment comprises cyclosporin A, Anise oil, and a Carbomer.

Another embodiment comprises cyclosporin A, Castor oil, and a Carbomer.

Another embodiment comprises cyclosporin A, Clove oil, and a Carbomer.

Another embodiment comprises cyclosporin A, Cassia oil, and a Carbomer.

Another embodiment comprises cyclosporin A, Cinnamon oil, and a Carbomer.

Another embodiment comprises cyclosporin A, an oil having a specific gravity from 0.90 to 0.95, and a Carbomer.

Another embodiment comprises cyclosporin A, Almond oil, and a Carbomer.

Another embodiment comprises cyclosporin A, Corn oil, and a Carbomer.

Another embodiment comprises cyclosporin A, Arachis oil, and a Carbomer.

Another embodiment comprises cyclosporin A, Cottonseed oil, and a Carbomer.

Another embodiment comprises cyclosporin A, Safflower oil, and a Carbomer.

Another embodiment comprises cyclosporin A, Maize oil, and a Carbomer.

Another embodiment comprises cyclosporin A, Linseed oil, and a Carbomer.

Another embodiment comprises cyclosporin A, Rapeseed oil, and a Carbomer.

Another embodiment comprises cyclosporin A, Soybean oil, and a Carbomer.

Another embodiment comprises cyclosporin A, Olive oil, and a Carbomer.

Another embodiment comprises cyclosporin A, Caraway oil, and a Carbomer.

Another embodiment comprises cyclosporin A, Rosemary oil, and a Carbomer.

Another embodiment comprises cyclosporin A, Peanut oil, and a Carbomer.

Another embodiment comprises cyclosporin A, Peppermint oil, and a Carbomer.

Another embodiment comprises cyclosporin A, Sunflower oil, and a Carbomer.

Another embodiment comprises cyclosporin A, Eucalyptus oil, and a Carbomer.

Another embodiment comprises cyclosporin A, Sesame oil, and a Carbomer.

Another embodiment comprises cyclosporin A, an oil having a specific gravity below 0.9, and a Carbomer.

Another embodiment comprises cyclosporin A, Mineral oil, and a Carbomer.

Another embodiment comprises cyclosporin A, Coriander oil, and a Carbomer.

Another embodiment comprises cyclosporin A, Lavender oil, and a Carbomer.

Another embodiment comprises cyclosporin A, Citronella oil, and a Carbomer.

Another embodiment comprises cyclosporin A, Juniper oil, and a Carbomer.

Another embodiment comprises cyclosporin A, Lemon oil, and a Carbomer.

Another embodiment comprises cyclosporin A, Orange oil, and a Carbomer.

Another embodiment comprises cyclosporin A, Clary sage oil, and a Carbomer.

Another embodiment comprises cyclosporin A, Nutmeg oil, and a Carbomer.

Another embodiment comprises cyclosporin A, Tea tree oil, and a Carbomer.

Another embodiment comprises cyclosporin A, an oil having a specific gravity from 0.95 to 1.07, and a phosphalipid.

Another embodiment comprises cyclosporin A, Anise oil, and a phosphalipid.

Another embodiment comprises cyclosporin A, Castor oil, and a phosphalipid.

Another embodiment comprises cyclosporin A, Clove oil, and a phosphalipid.

Another embodiment comprises cyclosporin A, Cassia oil, and a phosphalipid.

Another embodiment comprises cyclosporin A, Cinnamon oil, and a phosphalipid.

Another embodiment comprises cyclosporin A, an oil having a specific gravity from 0.90 to 0.95, and a phosphalipid.

Another embodiment comprises cyclosporin A, Almond oil, and a phosphalipid.

Another embodiment comprises cyclosporin A, Corn oil, and a phosphalipid.

Another embodiment comprises cyclosporin A, Arachis oil, and a phosphalipid.

Another embodiment comprises cyclosporin A, Cottonseed oil, and a phosphalipid.

Another embodiment comprises cyclosporin A, Safflower oil, and a phosphalipid.

Another embodiment comprises cyclosporin A, Maize oil, and a phosphalipid.

Another embodiment comprises cyclosporin A, Linseed oil, and a phosphalipid.

Another embodiment comprises cyclosporin A, Rapeseed oil, and a phosphalipid.

Another embodiment comprises cyclosporin A, Soybean oil, and a phosphalipid.

Another embodiment comprises cyclosporin A, Olive oil, and a phosphalipid.

Another embodiment comprises cyclosporin A, Caraway oil, and a phosphalipid.

Another embodiment comprises cyclosporin A, Rosemary oil, and a phosphalipid.

Another embodiment comprises cyclosporin A, Peanut oil, and a phosphalipid.

Another embodiment comprises cyclosporin A, Peppermint oil, and a phosphalipid.

Another embodiment comprises cyclosporin A, Sunflower oil, and a phosphalipid.

Another embodiment comprises cyclosporin A, Eucalyptus oil, and a phosphalipid.

Another embodiment comprises cyclosporin A, Sesame oil, and a phosphalipid.

Another embodiment comprises cyclosporin A, an oil having a specific gravity below 0.9, and a phosphalipid.

Another embodiment comprises cyclosporin A, Mineral oil, and a phosphalipid.

Another embodiment comprises cyclosporin A, Coriander oil, and a phosphalipid.

Another embodiment comprises cyclosporin A, Lavender oil, and a phosphalipid.

Another embodiment comprises cyclosporin A, Citronella oil, and a phosphalipid.

Another embodiment comprises cyclosporin A, Juniper oil, and a phosphalipid.

Another embodiment comprises cyclosporin A, Lemon oil, and a phosphalipid.

Another embodiment comprises cyclosporin A, Orange oil, and a phosphalipid.

Another embodiment comprises cyclosporin A, Clary sage oil, and a phosphalipid.

Another embodiment comprises cyclosporin A, Nutmeg oil, and a phosphalipid.

Another embodiment comprises cyclosporin A, Tea tree oil, and a phosphalipid.

Another embodiment comprises cyclosporin A, an oil having a specific gravity from 0.95 to 1.07, and phosphatidyl chloline.

Another embodiment comprises cyclosporin A, Anise oil, and phosphatidyl chloline.

Another embodiment comprises cyclosporin A, Castor oil, and phosphatidyl chloline.

Another embodiment comprises cyclosporin A, Clove oil, and phosphatidyl chloline.

Another embodiment comprises cyclosporin A, Cassia oil, and phosphatidyl chloline.

Another embodiment comprises cyclosporin A, Cinnamon oil, and phosphatidyl chloline.

Another embodiment comprises cyclosporin A, an oil having a specific gravity from 0.90 to 0.95, and phosphatidyl chloline.

Another embodiment comprises cyclosporin A, Almond oil, and phosphatidyl chloline.

Another embodiment comprises cyclosporin A, Corn oil, and phosphatidyl chloline.

Another embodiment comprises cyclosporin A, Arachis oil, and phosphatidyl chlorine.

Another embodiment comprises cyclosporin A, Cottonseed oil, and phosphatidyl chloline.

Another embodiment comprises cyclosporin A, Safflower oil, and phosphatidyl chloline.

Another embodiment comprises cyclosporin A, Maize oil, and phosphatidyl chIoline.

Another embodiment comprises cyclosporin A, Linseed oil, and phosphatidyl chloline.

Another embodiment comprises cyclosporin A, Rapeseed oil, and phosphatidyl chloline.

Another embodiment comprises cyclosporin A, Soybean oil, and phosphatidyl chloline.

Another embodiment comprises cyclosporin A, Olive oil, and phosphatidyl chloline.

Another embodiment comprises cyclosporin A, Caraway oil, and phosphatidyl chloline.

Another embodiment comprises cyclosporin A, Rosemary oil, and phosphatidyl chloline.

Another embodiment comprises cyclosporin A, Peanut oil, and phosphatidyl chloline.

Another embodiment comprises cyclosporin A, Peppermint oil, and phosphatidyl chloline.

Another embodiment comprises cyclosporin A, Sunflower oil, and phosphatidyl chloline.

Another embodiment comprises cyclosporin A, Eucalyptus oil, and phosphatidyl chloline.

Another embodiment comprises cyclosporin A, Sesame oil, and phosphatidyl chloline.

Another embodiment comprises cyclosporin A, an oil having a specific gravity below 0.9, and phosphatidyl chloline.

Another embodiment comprises cyclosporin A, Mineral oil, and phosphatidyl chloline.

Another embodiment comprises cyclosporin A, Coriander oil, and phosphatidyl chloline.

Another embodiment comprises cyclosporin A, Lavender oil, and phosphatidyl chloline.

Another embodiment comprises cyclosporin A, Citronella oil, and phosphatidyl chloline.

Another embodiment comprises cyclosporin A, Juniper oil, and phosphatidyl chloline.

Another embodiment comprises cyclosporin A, Lemon oil, and phosphatidyl chloline.

Another embodiment comprises cyclosporin A, Orange oil, and phosphatidyl chloline.

Another embodiment comprises cyclosporin A, Clary sage oil, and phosphatidyl chloline.

Another embodiment comprises cyclosporin A, Nutmeg oil, and phosphatidyl chloline.

Another embodiment comprises cyclosporin A, Tea tree oil, and phosphatidyl chloline.

Another embodiment comprises cyclosporin A, an oil having a specific gravity from 0.95 to 1.07, and phosphatidyl serine.

Another embodiment comprises cyclosporin A, Anise oil, and phosphatidyl serine.

Another embodiment comprises cyclosporin A, Castor oil, and phosphatidyl serine.

Another embodiment comprises cyclosporin A, Clove oil, and phosphatidyl serine.

Another embodiment comprises cyclosporin A, Cassia oil, and phosphatidyl serine.

Another embodiment comprises cyclosporin A, Cinnamon oil, and phosphatidyl serine.

Another embodiment comprises cyclosporin A, an oil having a specific gravity from 0.90 to 0.95, and phosphatidyl serine.

Another embodiment comprises cyclosporin A, Almond oil, and phosphatidyl serine.

Another embodiment comprises cyclosporin A, Corn oil, and phosphatidyl serine.

Another embodiment comprises cyclosporin A, Arachis oil, and phosphatidyl serine.

Another embodiment comprises cyclosporin A, Cottonseed oil, and phosphatidyl serine.

Another embodiment comprises cyclosporin A, Safflower oil, and phosphatidyl serine.

Another embodiment comprises cyclosporin A, Maize oil, and phosphatidyl serine.

Another embodiment comprises cyclosporin A, Linseed oil, and phosphatidyl serine.

Another embodiment comprises cyclosporin A, Rapeseed oil, and phosphatidyl serine.

Another embodiment comprises cyclosporin A, Soybean oil, and phosphatidyl serine.

Another embodiment comprises cyclosporin A, Olive oil, and phosphatidyl serine.

Another embodiment comprises cyclosporin A, Caraway oil, and phosphatidyl serine.

Another embodiment comprises cyclosporin A, Rosemary oil, and phosphatidyl serine.

Another embodiment comprises cyclosporin A, Peanut oil, and phosphatidyl serine.

Another embodiment comprises cyclosporin A, Peppermint oil, and phosphatidyl serine.

Another embodiment comprises cyclosporin A, Sunflower oil, and phosphatidyl serine.

Another embodiment comprises cyclosporin A, Eucalyptus oil, and phosphatidyl serine.

Another embodiment comprises cyclosporin A, Sesame oil, and phosphatidyl serine.

Another embodiment comprises cyclosporin A, an oil having a specific gravity below 0.9, and phosphatidyl serine.

Another embodiment comprises cyclosporin A, Mineral oil, and phosphatidyl serine.

Another embodiment comprises cyclosporin A, Coriander oil, and phosphatidyl serine.

Another embodiment comprises cyclosporin A, Lavender oil, and phosphatidyl serine.

Another embodiment comprises cyclosporin A, Citronella oil, and phosphatidyl serine.

Another embodiment comprises cyclosporin A, Juniper oil, and phosphatidyl serine.

Another embodiment comprises cyclosporin A, Lemon oil, and phosphatidyl serine.

Another embodiment comprises cyclosporin A, Orange oil, and phosphatidyl serine.

Another embodiment comprises cyclosporin A, Clary sage oil, and phosphatidyl serine.

Another embodiment comprises cyclosporin A, Nutmeg oil, and phosphatidyl serine.

Another embodiment comprises cyclosporin A, Tea tree oil, and phosphatidyl serine.

Another embodiment comprises from 0.005% to 0.05% cyclosporin A, an oil having a specific gravity from 0.95 to 1.07, and a sorbitan ester.

Another embodiment comprises from 0.005% to 0.05% cyclosporin A, Anise oil, and a sorbitan ester.

Another embodiment comprises from 0.005% to 0.05% cyclosporin A, Castor oil, and a sorbitan ester.

Another embodiment comprises from 0.005% to 0.05% cyclosporin A, Clove oil, and a sorbitan ester.

Another embodiment comprises from 0.005% to 0.05% cyclosporin A, Cassia oil, and a sorbitan ester.

Another embodiment comprises from 0.005% to 0.05% cyclosporin A, Cinnamon oil, and a sorbitan ester.

Another embodiment comprises from 0.005% to 0.05% cyclosporin A, an oil having a specific gravity from 0.90 to 0.95, and a sorbitan ester.

Another embodiment comprises from 0.005% to 0.05% cyclosporin A, Almond oil, and a sorbitan ester.

Another embodiment comprises from 0.005% to 0.05% cyclosporin A, Corn oil, and a sorbitan ester.

Another embodiment comprises from 0.005% to 0.05% cyclosporin A, Arachis oil, and a sorbitan ester.

Another embodiment comprises from 0.005% to 0.05% cyclosporin A, Cottonseed oil, and a sorbitan ester.

Another embodiment comprises from 0.005% to 0.05% cyclosporin A, Safflower oil, and a sorbitan ester.

Another embodiment comprises from 0.005% to 0.05% cyclosporin A, Maize oil, and a sorbitan ester.

Another embodiment comprises from 0.005% to 0.05% cyclosporin A, Linseed oil, and a sorbitan ester.

Another embodiment comprises from 0.005% to 0.05% cyclosporin A, Rapeseed oil, and a sorbitan ester.

Another embodiment comprises from 0.005% to 0.05% cyclosporin A, Soybean oil, and a sorbitan ester.

Another embodiment comprises from 0.005% to 0.05% cyclosporin A, Olive oil, and a sorbitan ester.

Another embodiment comprises from 0.005% to 0.05% cyclosporin A, Caraway oil, and a sorbitan ester.

Another embodiment comprises from 0.005% to 0.05% cyclosporin A, Rosemary oil, and a sorbitan ester.

Another embodiment comprises from 0.005% to 0.05% cyclosporin A, Peanut oil, and a sorbitan ester.

Another embodiment comprises from 0.005% to 0.05% cyclosporin A, Peppermint oil, and a sorbitan ester.

Another embodiment comprises from 0.005% to 0.05% cyclosporin A, Sunflower oil, and a sorbitan ester.

Another embodiment comprises from 0.005% to 0.05% cyclosporin A, Eucalyptus oil, and a sorbitan ester.

Another embodiment comprises from 0.005% to 0.05% cyclosporin A, Sesame oil, and a sorbitan ester.

Another embodiment comprises from 0.005% to 0.05% cyclosporin A, an oil having a specific gravity below 0.9, and a sorbitan ester.

Another embodiment comprises from 0.005% to 0.05% cyclosporin A, Mineral oil, and a sorbitan ester.

Another embodiment comprises from 0.005% to 0.05% cyclosporin A, Coriander oil, and a sorbitan ester.

Another embodiment comprises from 0.005% to 0.05% cyclosporin A, Lavender oil, and a sorbitan ester.

Another embodiment comprises from 0.005% to 0.05% cyclosporin A, Citronella oil, and a sorbitan ester.

Another embodiment comprises from 0.005% to 0.05% cyclosporin A, Juniper oil, and a sorbitan ester.

Another embodiment comprises from 0.005% to 0.05% cyclosporin A, Lemon oil, and a sorbitan ester.

Another embodiment comprises from 0.005% to 0.05% cyclosporin A, Orange oil, and a sorbitan ester.

Another embodiment comprises from 0.005% to 0.05% cyclosporin A, Clary sage oil, and a sorbitan ester.

Another embodiment comprises from 0.005% to 0.05% cyclosporin A, Nutmeg oil, and a sorbitan ester.

Another embodiment comprises from 0.005% to 0.05% cyclosporin A, Tea tree oil, and a sorbitan ester.

Another embodiment comprises from 0.005% to 0.05% cyclosporin A, an oil having a specific gravity from 0.95 to 1.07, and Polysorbate 80.

Another embodiment comprises from 0.005% to 0.05% cyclosporin A, Anise oil, and Polysorbate 80.

Another embodiment comprises from 0.005% to 0.05% cyclosporin A, Clove oil, and Polysorbate 80.

Another embodiment comprises from 0.005% to 0.05% cyclosporin A, Cassia oil, and Polysorbate 80.

Another embodiment comprises from 0.005% to 0.05% cyclosporin A, Cinnamon oil, and Polysorbate 80.

Another embodiment comprises from 0.005% to 0.05% cyclosporin A, an oil having a specific gravity from 0.90 to 0.95, and Polysorbate 80.

Another embodiment comprises from 0.005% to 0.05% cyclosporin A, Almond oil, and Polysorbate 80.

Another embodiment comprises from 0.005% to 0.05% cyclosporin A, Corn oil, and Polysorbate 80.

Another embodiment comprises from 0.005% to 0.05% cyclosporin A, Arachis oil, and Polysorbate 80.

Another embodiment comprises from 0.005% to 0.05% cyclosporin A, Cottonseed oil, and Polysorbate 80.

Another embodiment comprises from 0.005% to 0.05% cyclosporin A, Safflower oil, and Polysorbate 80.

Another embodiment comprises from 0.005% to 0.05% cyclosporin A, Maize oil, and Polysorbate 80.

Another embodiment comprises from 0.005% to 0.05% cyclosporin A, Linseed oil, and Polysorbate 80.

Another embodiment comprises from 0.005% to 0.05% cyclosporin A, Rapeseed oil, and Polysorbate 80.

Another embodiment comprises from 0.005% to 0.05% cyclosporin A, Soybean oil, and Polysorbate 80.

Another embodiment comprises from 0.005% to 0.05% cyclosporin A, Olive oil, and Polysorbate 80.

Another embodiment comprises from 0.005% to 0.05% cyclosporin A, Caraway oil, and Polysorbate 80.

Another embodiment comprises from 0.005% to 0.05% cyclosporin A, Rosemary oil, and Polysorbate 80.

Another embodiment comprises from 0.005% to 0.05% cyclosporin A, Peanut oil, and Polysorbate 80.

Another embodiment comprises from 0.005% to 0.05% cyclosporin A, Peppermint oil, and Polysorbate 80.

Another embodiment comprises from 0.005% to 0.05% cyclosporin A, Sunflower oil, and Polysorbate 80.

Another embodiment comprises from 0.005% to 0.05% cyclosporin A, Eucalyptus oil, and Polysorbate 80.

Another embodiment comprises from 0.005% to 0.05% cyclosporin A, Sesame oil, and Polysorbate 80.

Another embodiment comprises from 0.005% to 0.05% cyclosporin A, an oil having a specific gravity below 0.9, and Polysorbate 80.

Another embodiment comprises from 0.005% to 0.05% cyclosporin A, Mineral oil, and Polysorbate 80.

Another embodiment comprises from 0.005% to 0.05% cyclosporin A, Coriander oil, and Polysorbate 80.

Another embodiment comprises from 0.005% to 0.05% cyclosporin A, Lavender oil, and Polysorbate 80.

Another embodiment comprises from 0.005% to 0.05% cyclosporin A, Citronella oil, and Polysorbate 80.

Another embodiment comprises from 0.005% to 0.05% cyclosporin A, Juniper oil, and Polysorbate 80.

Another embodiment comprises from 0.005% to 0.05% cyclosporin A, Lemon oil, and Polysorbate 80.

Another embodiment comprises from 0.005% to 0.05% cyclosporin A, Orange oil, and Polysorbate 80.

Another embodiment comprises from 0.005% to 0.05% cyclosporin A, Clary sage oil, and Polysorbate 80.

Another embodiment comprises from 0.005% to 0.05% cyclosporin A, Nutmeg oil, and Polysorbate 80.

Another embodiment comprises from 0.005% to 0.05% cyclosporin A, Tea tree oil, and Polysorbate 80.

Another embodiment comprises from 0.005% to 0.05% cyclosporin A, an oil having a specific gravity from 0.95 to 1.07, and Polysorbate 80.

Another embodiment comprises from 0.005% to 0.05% cyclosporin A, Anise oil, and Polysorbate 80.

Another embodiment comprises from 0.005% to 0.05% cyclosporin A, Castor oil, and Polysorbate 80.

Another embodiment comprises from 0.005% to 0.05% cyclosporin A, Clove oil, and Polysorbate 80.

Another embodiment comprises from 0.005% to 0.05% cyclosporin A, Cassia oil, and Polysorbate 80.

Another embodiment comprises from 0.005% to 0.05% cyclosporin A, Cinnamon oil, and Polysorbate 80.

Another embodiment comprises from 0.005% to 0.05% cyclosporin A, an oil having a specific gravity from 0.90 to 0.95, and Polysorbate 80.

Another embodiment comprises from 0.005% to 0.05% cyclosporin A, Almond oil, and Polysorbate 80.

Another embodiment comprises from 0.005% to 0.05% cyclosporin A, Corn oil, and Polysorbate 80.

Another embodiment comprises from 0.005% to 0.05% cyclosporin A, Arachis oil, and Polysorbate 80.

Another embodiment comprises from 0.005% to 0.05% cyclosporin A, Cottonseed oil, and Polysorbate 80.

Another embodiment comprises from 0.005% to 0.05% cyclosporin A, Safflower oil, and Polysorbate 80.

Another embodiment comprises from 0.005% to 0.05% cyclosporin A, Maize oil, and Polysorbate 80.

Another embodiment comprises from 0.005% to 0.05% cyclosporin A, Linseed oil, and Polysorbate 80.

Another embodiment comprises from 0.005% to 0.05% cyclosporin A, Rapeseed oil, and Polysorbate 80.

Another embodiment comprises from 0.005% to 0.05% cyclosporin A, Soybean oil, and Polysorbate 80.

Another embodiment comprises from 0.005% to 0.05% cyclosporin A, Olive oil, and Polysorbate 80.

Another embodiment comprises from 0.005% to 0.05% cyclosporin A, Caraway oil, and Polysorbate 80.

Another embodiment comprises from 0.005% to 0.05% cyclosporin A, Rosemary oil, and Polysorbate 80.

Another embodiment comprises from 0.005% to 0.05% cyclosporin A, Peanut oil, and Polysorbate 80.

Another embodiment comprises from 0.005% to 0.05% cyclosporin A, Peppermint oil, and Polysorbate 80.

Another embodiment comprises from 0.005% to 0.05% cyclosporin A, Sunflower oil, and Polysorbate 80.

Another embodiment comprises from 0.005% to 0.05% cyclosporin A, Eucalyptus oil, and Polysorbate 80.

Another embodiment comprises from 0.005% to 0.05% cyclosporin A, Sesame oil, and Polysorbate 80.

Another embodiment comprises from 0.005% to 0.05% cyclosporin A, an oil having a specific gravity below 0.9, and Polysorbate 80.

Another embodiment comprises from 0.005% to 0.05% cyclosporin A, Mineral oil, and Polysorbate 80.

Another embodiment comprises from 0.005% to 0.05% cyclosporin A, Coriander oil, and Polysorbate 80.

Another embodiment comprises from 0.005% to 0.05% cyclosporin A, Lavender oil, and Polysorbate 80.

Another embodiment comprises from 0.005% to 0.05% cyclosporin A, Citronella oil, and Polysorbate 80.

Another embodiment comprises from 0.005% to 0.05% cyclosporin A, Juniper oil, and Polysorbate 80.

Another embodiment comprises from 0.005% to 0.05% cyclosporin A, Lemon oil, and Polysorbate 80.

Another embodiment comprises from 0.005% to 0.05% cyclosporin A, Orange oil, and Polysorbate 80.

Another embodiment comprises from 0.005% to 0.05% cyclosporin A, Clary sage oil, and Polysorbate 80.

Another embodiment comprises from 0.005% to 0.05% cyclosporin A, Nutmeg oil, and Polysorbate 80.

Another embodiment comprises from 0.005% to 0.05% cyclosporin A, Tea tree oil, and Polysorbate 80.

Another embodiment comprises from 0.005% to 0.05% cyclosporin A, an oil having a specific gravity from 0.95 to 1.07, and Polysorbate 60.

Another embodiment comprises from 0.005% to 0.05% cyclosporin A, Anise oil, and Polysorbate 60.

Another embodiment comprises from 0.005% to 0.05% cyclosporin A, Castor oil, and Polysorbate 60.

Another embodiment comprises from 0.005% to 0.05% cyclosporin A, Clove oil, and Polysorbate 60.

Another embodiment comprises from 0.005% to 0.05% cyclosporin A, Cassia oil, and Polysorbate 60.

Another embodiment comprises from 0.005% to 0.05% cyclosporin A, Cinnamon oil, and Polysorbate 60.

Another embodiment comprises from 0.005% to 0.05% cyclosporin A, an oil having a specific gravity from 0.90 to 0.95, and Polysorbate 60.

Another embodiment comprises from 0.005% to 0.05% cyclosporin A, Almond oil, and Polysorbate 60.

Another embodiment comprises from 0.005% to 0.05% cyclosporin A, Corn oil, and Polysorbate 60.

Another embodiment comprises from 0.005% to 0.05% cyclosporin A, Arachis oil, and Polysorbate 60.

Another embodiment comprises from 0.005% to 0.05% cyclosporin A, Cottonseed oil, and Polysorbate 60.

Another embodiment comprises from 0.005% to 0.05% cyclosporin A, Safflower oil, and Polysorbate 60.

Another embodiment comprises from 0.005% to 0.05% cyclosporin A, Maize oil, and Polysorbate 60.

Another embodiment comprises from 0.005% to 0.05% cyclosporin A, Linseed oil, and Polysorbate 60.

Another embodiment comprises from 0.005% to 0.05% cyclosporin A, Rapeseed oil, and Polysorbate 60.

Another embodiment comprises from 0.005% to 0.05% cyclosporin A, Soybean oil, and Polysorbate 60.

Another embodiment comprises from 0.005% to 0.05% cyclosporin A, Olive oil, and Polysorbate 60.

Another embodiment comprises from 0.005% to 0.05% cyclosporin A, Caraway oil, and Polysorbate 60.

Another embodiment comprises from 0.005% to 0.05% cyclosporin A, Rosemary oil, and Polysorbate 60.

Another embodiment comprises from 0.005% to 0.05% cyclosporin A, Peanut oil, and Polysorbate 60.

Another embodiment comprises from 0.005% to 0.05% cyclosporin A, Peppermint oil, and Polysorbate 60.

Another embodiment comprises from 0.005% to 0.05% cyclosporin A, Sunflower oil, and Polysorbate 60.

Another embodiment comprises from 0.005% to 0.05% cyclosporin A, Eucalyptus oil, and Polysorbate 60.

Another embodiment comprises from 0.005% to 0.05% cyclosporin A, Sesame oil, and Polysorbate 60.

Another embodiment comprises from 0.005% to 0.05% cyclosporin A, an oil having a specific gravity below 0.9, and Polysorbate 60.

Another embodiment comprises from 0.005% to 0.05% cyclosporin A, Mineral oil, and Polysorbate 60.

Another embodiment comprises from 0.005% to 0.05% cyclosporin A, Coriander oil, and Polysorbate 60.

Another embodiment comprises from 0.005% to 0.05% cyclosporin A, Lavender oil, and Polysorbate 60.

Another embodiment comprises from 0.005% to 0.05% cyclosporin A, Citronella oil, and Polysorbate 60.

Another embodiment comprises from 0.005% to 0.05% cyclosporin A, Juniper oil, and Polysorbate 60.

Another embodiment comprises from 0.005% to 0.05% cyclosporin A, Lemon oil, and Polysorbate 60.

Another embodiment comprises from 0.005% to 0.05% cyclosporin A, Orange oil, and Polysorbate 60.

Another embodiment comprises from 0.005% to 0.05% cyclosporin A, Clary sage oil, and Polysorbate 60.

Another embodiment comprises from 0.005% to 0.05% cyclosporin A, Nutmeg oil, and Polysorbate 60.

Another embodiment comprises from 0.005% to 0.05% cyclosporin A, Tea tree oil, and Polysorbate 60.

Another embodiment comprises from 0.005% to 0.05% cyclosporin A, an oil having a specific gravity from 0.95 to 1.07, and Polysorbate 40.

Another embodiment comprises from 0.005% to 0.05% cyclosporin A, Anise oil, and Polysorbate 40.

Another embodiment comprises from 0.005% to 0.05% cyclosporin A, Castor oil, and Polysorbate 40.

Another embodiment comprises from 0.005% to 0.05% cyclosporin A, Clove oil, and Polysorbate 40.

Another embodiment comprises from 0.005% to 0.05% cyclosporin A, Cassia oil, and Polysorbate 40.

Another embodiment comprises from 0.005% to 0.05% cyclosporin A, Cinnamon oil, and Polysorbate 40.

Another embodiment comprises from 0.005% to 0.05% cyclosporin A, an oil having a specific gravity from 0.90 to 0.95, and Polysorbate 40.

Another embodiment comprises from 0.005% to 0.05% cyclosporin A, Almond oil, and Polysorbate 40.

Another embodiment comprises from 0.005% to 0.05% cyclosporin A, Corn oil, and Polysorbate 40.

Another embodiment comprises from 0.005% to 0.05% cyclosporin A, Arachis oil, and Polysorbate 40.

Another embodiment comprises from 0.005% to 0.05% cyclosporin A, Cottonseed oil, and Polysorbate 40.

Another embodiment comprises from 0.005% to 0.05% cyclosporin A, Safflower oil, and Polysorbate 40.

Another embodiment comprises from 0.005% to 0.05% cyclosporin A, Maize oil, and Polysorbate 40.

Another embodiment comprises from 0.005% to 0.05% cyclosporin A, Linseed oil, and Polysorbate 40.

Another embodiment comprises from 0.005% to 0.05% cyclosporin A, Rapeseed oil, and Polysorbate 40.

Another embodiment comprises from 0.005% to 0.05% cyclosporin A, Soybean oil, and Polysorbate 40.

Another embodiment comprises from 0.005% to 0.05% cyclosporin A, Olive oil, and Polysorbate 40.

Another embodiment comprises from 0.005% to 0.05% cyclosporin A, Caraway oil, and Polysorbate 40.

Another embodiment comprises from 0.005% to 0.05% cyclosporin A, Rosemary oil, and Polysorbate 40.

Another embodiment comprises from 0.005% to 0.05% cyclosporin A, Peanut oil, and Polysorbate 40.

Another embodiment comprises from 0.005% to 0.05% cyclosporin A, Peppermint oil, and Polysorbate 40.

Another embodiment comprises from 0.005% to 0.05% cyclosporin A, Sunflower oil, and Polysorbate 40.

Another embodiment comprises from 0.005% to 0.05% cyclosporin A, Eucalyptus oil, and Polysorbate 40.

Another embodiment comprises from 0.005% to 0.05% cyclosporin A, Sesame oil, and Polysorbate 40.

Another embodiment comprises from 0.005% to 0.05% cyclosporin A, an oil having a specific gravity below 0.9, and Polysorbate 40.

Another embodiment comprises from 0.005% to 0.05% cyclosporin A, Mineral oil, and Polysorbate 40.

Another embodiment comprises from 0.005% to 0.05% cyclosporin A, Coriander oil, and Polysorbate 40.

Another embodiment comprises from 0.005% to 0.05% cyclosporin A, Lavender oil, and Polysorbate 40.

Another embodiment comprises from 0.005% to 0.05% cyclosporin A, Citronella oil, and Polysorbate 40.

Another embodiment comprises from 0.005% to 0.05% cyclosporin A, Juniper oil, and Polysorbate 40.

Another embodiment comprises from 0.005% to 0.05% cyclosporin A, Lemon oil, and Polysorbate 40.

Another embodiment comprises from 0.005% to 0.05% cyclosporin A, Orange oil, and Polysorbate 40.

Another embodiment comprises from 0.005% to 0.05% cyclosporin A, Clary sage oil, and Polysorbate 40.

Another embodiment comprises from 0.005% to 0.05% cyclosporin A, Nutmeg oil, and Polysorbate 40.

Another embodiment comprises from 0.005% to 0.05% cyclosporin A, Tea tree oil, and Polysorbate 40.

Another embodiment comprises from 0.005% to 0.05% cyclosporin A, an oil having a specific gravity from 0.95 to 1.07, and Polysorbate 20.

Another embodiment comprises from 0.005% to 0.05% cyclosporin A, Anise oil, and Polysorbate 20.

Another embodiment comprises from 0.005% to 0.05% cyclosporin A, Castor oil, and Polysorbate 20.

Another embodiment comprises from 0.005% to 0.05% cyclosporin A, Clove oil, and Polysorbate 20.

Another embodiment comprises from 0.005% to 0.05% cyclosporin A, Cassia oil, and Polysorbate 20.

Another embodiment comprises from 0.005% to 0.05% cyclosporin A, Cinnamon oil, and Polysorbate 20.

Another embodiment comprises from 0.005% to 0.05% cyclosporin A, an oil having a specific gravity from 0.90 to 0.95, and Polysorbate 20.

Another embodiment comprises from 0.005% to 0.05% cyclosporin A, Almond oil, and Polysorbate 20.

Another embodiment comprises from 0.005% to 0.05% cyclosporin A, Corn oil, and Polysorbate 20.

Another embodiment comprises from 0.005% to 0.05% cyclosporin A, Arachis oil, and Polysorbate 20.

Another embodiment comprises from 0.005% to 0.05% cyclosporin A, Cottonseed oil, and Polysorbate 20.

Another embodiment comprises from 0.005% to 0.05% cyclosporin A, Safflower oil, and Polysorbate 20.

Another embodiment comprises from 0.005% to 0.05% cyclosporin A, Maize oil, and Polysorbate 20.

Another embodiment comprises from 0.005% to 0.05% cyclosporin A, Linseed oil, and Polysorbate 20.

Another embodiment comprises from 0.005% to 0.05% cyclosporin A, Rapeseed oil, and Polysorbate 20.

Another embodiment comprises from 0.005% to 0.05% cyclosporin A, Soybean oil, and Polysorbate 20.

Another embodiment comprises from 0.005% to 0.05% cyclosporin A, Olive oil, and Polysorbate 20.

Another embodiment comprises from 0.005% to 0.05% cyclosporin A, Caraway oil, and Polysorbate 20.

Another embodiment comprises from 0.005% to 0.05% cyclosporin A, Rosemary oil, and Polysorbate 20.

Another embodiment comprises from 0.005% to 0.05% cyclosporin A, Peanut oil, and Polysorbate 20.

Another embodiment comprises from 0.005% to 0.05% cyclosporin A, Peppermint oil, and Polysorbate 20.

Another embodiment comprises from 0.005% to 0.05% cyclosporin A, Sunflower oil, and Polysorbate 20.

Another embodiment comprises from 0.005% to 0.05% cyclosporin A, Eucalyptus oil, and Polysorbate 20.

Another embodiment comprises from 0.005% to 0.05% cyclosporin A, Sesame oil, and Polysorbate 20.

Another embodiment comprises from 0.005% to 0.05% cyclosporin A, an oil having a specific gravity below 0.9, and Polysorbate 20.

Another embodiment comprises from 0.005% to 0.05% cyclosporin A, Mineral oil, and Polysorbate 20.

Another embodiment comprises from 0.005% to 0.05% cyclosporin A, Coriander oil, and Polysorbate 20.

Another embodiment comprises from 0.005% to 0.05% cyclosporin A, Lavender oil, and Polysorbate 20.

Another embodiment comprises from 0.005% to 0.05% cyclosporin A, Citronella oil, and Polysorbate 20.

Another embodiment comprises from 0.005% to 0.05% cyclosporin A, Juniper oil, and Polysorbate 20.

Another embodiment comprises from 0.005% to 0.05% cyclosporin A, Lemon oil, and Polysorbate 20.

Another embodiment comprises from 0.005% to 0.05% cyclosporin A, Orange oil, and Polysorbate 20.

Another embodiment comprises from 0.005% to 0.05% cyclosporin A, Clary sage oil, and Polysorbate 20.

Another embodiment comprises from 0.005% to 0.05% cyclosporin A, Nutmeg oil, and Polysorbate 20.

Another embodiment comprises from 0.005% to 0.05% cyclosporin A, Tea tree oil, and Polysorbate 20.

Another embodiment comprises from 0.005% to 0.05% cyclosporin A, an oil having a specific gravity from 0.95 to 1.07, and a stearate surfactant.

Another embodiment comprises from 0.005% to 0.05% cyclosporin A, Anise oil, and a stearate surfactant.

Another embodiment comprises from 0.005% to 0.05% cyclosporin A, Castor oil, and a stearate surfactant.

Another embodiment comprises from 0.005% to 0.05% cyclosporin A, Clove oil, and a stearate surfactant.

Another embodiment comprises from 0.005% to 0.05% cyclosporin A, Cassia oil, and a stearate surfactant.

Another embodiment comprises from 0.005% to 0.05% cyclosporin A, Cinnamon oil, and a stearate surfactant.

Another embodiment comprises from 0.005% to 0.05% cyclosporin A, an oil having a specific gravity from 0.90 to 0.95, and a stearate surfactant.

Another embodiment comprises from 0.005% to 0.05% cyclosporin A, Almond oil, and a stearate surfactant.

Another embodiment comprises from 0.005% to 0.05% cyclosporin A, Corn oil, and a stearate surfactant.

Another embodiment comprises from 0.005% to 0.05% cyclosporin A, Arachis oil, and a stearate surfactant.

Another embodiment comprises from 0.005% to 0.05% cyclosporin A, Cottonseed oil, and a stearate surfactant.

Another embodiment comprises from 0.005% to 0.05% cyclosporin A, Safflower oil, and a stearate surfactant.

Another embodiment comprises from 0.005% to 0.05% cyclosporin A, Maize oil, and a stearate surfactant.

Another embodiment comprises from 0.005% to 0.05% cyclosporin A, Linseed oil, and a stearate surfactant.

Another embodiment comprises from 0.005% to 0.05% cyclosporin A, Rapeseed oil, and a stearate surfactant.

Another embodiment comprises from 0.005% to 0.05% cyclosporin A, Soybean oil, and a stearate surfactant.

Another embodiment comprises from 0.005% to 0.05% cyclosporin A, Olive oil, and a stearate surfactant.

Another embodiment comprises from 0.005% to 0.05% cyclosporin A, Caraway oil, and a stearate surfactant.

Another embodiment comprises from 0.005% to 0.05% cyclosporin A, Rosemary oil, and a stearate surfactant.

Another embodiment comprises from 0.005% to 0.05% cyclosporin A, Peanut oil, and a stearate surfactant.

Another embodiment comprises from 0.005% to 0.05% cyclosporin A, Peppermint oil, and a stearate surfactant.

Another embodiment comprises from 0.005% to 0.05% cyclosporin A, Sunflower oil, and a stearate surfactant.

Another embodiment comprises from 0.005% to 0.05% cyclosporin A, Eucalyptus oil, and a stearate surfactant.

Another embodiment comprises from 0.005% to 0.05% cyclosporin A, Sesame oil, and a stearate surfactant.

Another embodiment comprises from 0.005% to 0.05% cyclosporin A, an oil having a specific gravity below 0.9, and a stearate surfactant.

Another embodiment comprises from 0.005% to 0.05% cyclosporin A, Mineral oil, and a stearate surfactant.

Another embodiment comprises from 0.005% to 0.05% cyclosporin A, Coriander oil, and a stearate surfactant.

Another embodiment comprises from 0.005% to 0.05% cyclosporin A, Lavender oil, and a stearate surfactant.

Another embodiment comprises from 0.005% to 0.05% cyclosporin A, Citronella oil, and a stearate surfactant.

Another embodiment comprises from 0.005% to 0.05% cyclosporin A, Juniper oil, and a stearate surfactant.

Another embodiment comprises from 0.005% to 0.05% cyclosporin A, Lemon oil, and a stearate surfactant.

Another embodiment comprises from 0.005% to 0.05% cyclosporin A, Orange oil, and a stearate surfactant.

Another embodiment comprises from 0.005% to 0.05% cyclosporin A, Clary sage oil, and a stearate surfactant.

Another embodiment comprises from 0.005% to 0.05% cyclosporin A, Nutmeg oil, and a stearate surfactant.

Another embodiment comprises from 0.005% to 0.05% cyclosporin A, Tea tree oil, and a stearate surfactant.

Another embodiment comprises from 0.005% to 0.05% cyclosporin A, an oil having a specific gravity from 0.95 to 1.07, and glyceryl stearate.

Another embodiment comprises from 0.005% to 0.05% cyclosporin A, Anise oil, and glyceryl stearate.

Another embodiment comprises from 0.005% to 0.05% cyclosporin A, Castor oil, and glyceryl stearate.

Another embodiment comprises from 0.005% to 0.05% cyclosporin A, Clove oil, and glyceryl stearate.

Another embodiment comprises from 0.005% to 0.05% cyclosporin A, Cassia oil, and glyceryl stearate.

Another embodiment comprises from 0.005% to 0.05% cyclosporin A, Cinnamon oil, and glyceryl stearate.

Another embodiment comprises from 0.005% to 0.05% cyclosporin A, an oil having a specific gravity from 0.90 to 0.95, and glyceryl stearate.

Another embodiment comprises from 0.005% to 0.05% cyclosporin A, Almond oil, and glyceryl stearate.

Another embodiment comprises from 0.005% to 0.05% cyclosporin A, Corn oil, and glyceryl stearate.

Another embodiment comprises from 0.005% to 0.05% cyclosporin A, Arachis oil, and glyceryl stearate.

Another embodiment comprises from 0.005% to 0.05% cyclosporin A, Cottonseed oil, and glyceryl stearate.

Another embodiment comprises from 0.005% to 0.05% cyclosporin A, Safflower oil, and glyceryl stearate.

Another embodiment comprises from 0.005% to 0.05% cyclosporin A, Maize oil, and glyceryl stearate.

Another embodiment comprises from 0.005% to 0.05% cyclosporin A, Linseed oil, and glyceryl stearate.

Another embodiment comprises from 0.005% to 0.05% cyclosporin A, Rapeseed oil, and glyceryl stearate.

Another embodiment comprises from 0.005% to 0.05% cyclosporin A, Soybean oil, and glyceryl stearate.

Another embodiment comprises from 0.005% to 0.05% cyclosporin A, Olive oil, and glyceryl stearate.

Another embodiment comprises from 0.005% to 0.05% cyclosporin A, Caraway oil, and glyceryl stearate.

Another embodiment comprises from 0.005% to 0.05% cyclosporin A, Rosemary oil, and glyceryl stearate.

Another embodiment comprises from 0.005% to 0.05% cyclosporin A, Peanut oil, and glyceryl stearate.

Another embodiment comprises from 0.005% to 0.05% cyclosporin A, Peppermint oil, and glyceryl stearate.

Another embodiment comprises from 0.005% to 0.05% cyclosporin A, Sunflower oil, and glyceryl stearate.

Another embodiment comprises from 0.005% to 0.05% cyclosporin A, Eucalyptus oil, and glyceryl stearate.

Another embodiment comprises from 0.005% to 0.05% cyclosporin A, Sesame oil, and glyceryl stearate.

Another embodiment comprises from 0.005% to 0.05% cyclosporin A, an oil having a specific gravity below 0.9, and glyceryl stearate.

Another embodiment comprises from 0.005% to 0.05% cyclosporin A, Mineral oil, and glyceryl stearate.

Another embodiment comprises from 0.005% to 0.05% cyclosporin A, Coriander oil, and glyceryl stearate.

Another embodiment comprises from 0.005% to 0.05% cyclosporin A, Lavender oil, and glyceryl stearate.

Another embodiment comprises from 0.005% to 0.05% cyclosporin A, Citronella oil, and glyceryl stearate.

Another embodiment comprises from 0.005% to 0.05% cyclosporin A, Juniper oil, and glyceryl stearate.

Another embodiment comprises from 0.005% to 0.05% cyclosporin A, Lemon oil, and glyceryl stearate.

Another embodiment comprises from 0.005% to 0.05% cyclosporin A, Orange oil, and glyceryl stearate.

Another embodiment comprises from 0.005% to 0.05% cyclosporin A, Clary sage oil, and glyceryl stearate.

Another embodiment comprises from 0.005% to 0.05% cyclosporin A, Nutmeg oil, and glyceryl stearate.

Another embodiment comprises from 0.005% to 0.05% cyclosporin A, Tea tree oil, and glyceryl stearate.

Another embodiment comprises from 0.005% to 0.05% cyclosporin A, an oil having a specific gravity from 0.95 to 1.07, and isopropyl stearate.

Another embodiment comprises from 0.005% to 0.05% cyclosporin A, Anise oil, and isopropyl stearate.

Another embodiment comprises from 0.005% to 0.05% cyclosporin A, Castor oil, and isopropyl stearate.

Another embodiment comprises from 0.005% to 0.05% cyclosporin A, Clove oil, and isopropyl stearate.

Another embodiment comprises from 0.005% to 0.05% cyclosporin A, Cassia oil, and isopropyl stearate.

Another embodiment comprises from 0.005% to 0.05% cyclosporin A, Cinnamon oil, and isopropyl stearate.

Another embodiment comprises from 0.005% to 0.05% cyclosporin A, an oil having a specific gravity from 0.90 to 0.95, and isopropyl stearate.

Another embodiment comprises from 0.005% to 0.05% cyclosporin A, Almond oil, and isopropyl stearate.

Another embodiment comprises from 0.005% to 0.05% cyclosporin A, Corn oil, and isopropyl stearate.

Another embodiment comprises from 0.005% to 0.05% cyclosporin A, Arachis oil, and isopropyl stearate.

Another embodiment comprises from 0.005% to 0.05% cyclosporin A, Cottonseed oil, and isopropyl stearate.

Another embodiment comprises from 0.005% to 0.05% cyclosporin A, Safflower oil, and isopropyl stearate.

Another embodiment comprises from 0.005% to 0.05% cyclosporin A, Maize oil, and isopropyl stearate.

Another embodiment comprises from 0.005% to 0.05% cyclosporin A, Linseed oil, and isopropyl stearate.

Another embodiment comprises from 0.005% to 0.05% cyclosporin A, Rapeseed oil, and isopropyl stearate.

Another embodiment comprises from 0.005% to 0.05% cyclosporin A, Soybean oil, and isopropyl stearate.

Another embodiment comprises from 0.005% to 0.05% cyclosporin A, Olive oil, and isopropyl stearate.

Another embodiment comprises from 0.005% to 0.05% cyclosporin A, Caraway oil, and isopropyl stearate.

Another embodiment comprises from 0.005% to 0.05% cyclosporin A, Rosemary oil, and isopropyl stearate.

Another embodiment comprises from 0.005% to 0.05% cyclosporin A, Peanut oil, and isopropyl stearate.

Another embodiment comprises from 0.005% to 0.05% cyclosporin A, Peppermint oil, and isopropyl stearate.

Another embodiment comprises from 0.005% to 0.05% cyclosporin A, Sunflower oil, and isopropyl stearate.

Another embodiment comprises from 0.005% to 0.05% cyclosporin A, Eucalyptus oil, and isopropyl stearate.

Another embodiment comprises from 0.005% to 0.05% cyclosporin A, Sesame oil, and isopropyl stearate.

Another embodiment comprises from 0.005% to 0.05% cyclosporin A, an oil.having a specific gravity below 0.9, and isopropyl stearate.

Another embodiment comprises from 0.005% to 0.05% cyclosporin A, Mineral oil, and isopropyl stearate.

Another embodiment comprises from 0.005% to 0.05% cyclosporin A, Coriander oil, and isopropyl stearate.

Another embodiment comprises from 0.005% to 0.05% cyclosporin A, Lavender oil, and isopropyl stearate.

Another embodiment comprises from 0.005% to 0.05% cyclosporin A, Citronella oil, and isopropyl stearate.

Another embodiment comprises from 0.005% to 0.05% cyclosporin A, Juniper oil, and isopropyl stearate.

Another embodiment comprises from 0.005% to 0.05% cyclosporin A, Lemon oil, and isopropyl stearate.

Another embodiment comprises from 0.005% to 0.05% cyclosporin A, Orange oil, and isopropyl stearate.

Another embodiment comprises from 0.005% to 0.05% cyclosporin A, Clary sage oil, and isopropyl stearate.

Another embodiment comprises from 0.005% to 0.05% cyclosporin A, Nutmeg oil, and isopropyl stearate.

Another embodiment comprises from 0.005% to 0.05% cyclosporin A, Tea tree oil, and isopropyl stearate.

Another embodiment comprises from 0.005% to 0.05% cyclosporin A, an oil having a specific gravity from 0.95 to 1.07, and polyoxyl stearate.

Another embodiment comprises from 0.005% to 0.05% cyclosporin A, Anise oil, and polyoxyl stearate.

Another embodiment comprises from 0.005% to 0.05% cyclosporin A, Castor oil, and polyoxyl stearate.

Another embodiment comprises from 0.005% to 0.05% cyclosporin A, Clove oil, and polyoxyl stearate.

Another embodiment comprises from 0.005% to 0.05% cyclosporin A, Cassia oil, and polyoxyl stearate.

Another embodiment comprises from 0.005% to 0.05% cyclosporin A, Cinnamon oil, and polyoxyl stearate.

Another embodiment comprises from 0.005% to 0.05% cyclosporin A, an oil having a specific gravity from 0.90 to 0.95, and polyoxyl stearate.

Another embodiment comprises from 0.005% to 0.05% cyclosporin A, Almond oil, and polyoxyl stearate.

Another embodiment comprises from 0.005% to 0.05% cyclosporin A, Corn oil, and polyoxyl stearate.

Another embodiment comprises from 0.005% to 0.05% cyclosporin A, Arachis oil, and polyoxyl stearate.

Another embodiment comprises from 0.005% to 0.05% cyclosporin A, Cottonseed oil, and polyoxyl stearate.

Another embodiment comprises from 0.005% to 0.05% cyclosporin A, Safflower oil, and polyoxyl stearate.

Another embodiment comprises from 0.005% to 0.05% cyclosporin A, Maize oil, and polyoxyl stearate.

Another embodiment comprises from 0.005% to 0.05% cyclosporin A, Linseed oil, and polyoxyl stearate.

Another embodiment comprises from 0.005% to 0.05% cyclosporin A, Rapeseed oil, and polyoxyl stearate.

Another embodiment comprises from 0.005% to 0.05% cyclosporin A, Soybean oil, and polybxyl stearate.

Another embodiment comprises from 0.005% to 0.05% cyclosporin A, Olive oil, and polyoxyl stearate.

Another embodiment comprises from 0.005% to 0.05% cyclosporin A, Caraway oil, and polyoxyl stearate.

Another embodiment comprises from 0.005% to 0.05% cyclosporin A, Rosemary oil, and polyoxyl stearate.

Another embodiment comprises from 0.005% to 0.05% cyclosporin A, Peanut oil, and polyoxyl stearate.

Another embodiment comprises from 0.005% to 0.05% cyclosporin A, Peppermint oil, and polyoxyl stearate.

Another embodiment comprises from 0.005% to 0.05% cyclosporin A, Sunflower oil, and polyoxyl stearate.

Another embodiment comprises from 0.005% to 0.05% cyclosporin A, Eucalyptus oil, and polyoxyl stearate.

Another embodiment comprises from 0.005% to 0.05% cyclosporin A, Sesame oil, and polyoxyl stearate.

Another embodiment comprises from 0.005% to 0.05% cyclosporin A, an oil having a specific gravity below 0.9, and polyoxyl stearate.

Another embodiment comprises from 0.005% to 0.05% cyclosporin A, Mineral oil, and polyoxyl stearate.

Another embodiment comprises from 0.005% to 0.05% cyclosporin A, Coriander oil, and polyoxyl stearate.

Another embodiment comprises from 0.005% to 0.05% cyclosporin A, Lavender oil, and polyoxyl stearate.

Another embodiment comprises from 0.005% to 0.05% cyclosporin A, Citronella oil, and polyoxyl stearate.

Another embodiment comprises from 0.005% to 0.05% cyclosporin A, Juniper oil, and polyoxyl stearate.

Another embodiment comprises from 0.005% to 0.05% cyclosporin A, Lemon oil, and polyoxyl stearate.

Another embodiment comprises from 0.005% to 0.05% cyclosporin A, Orange oil, and polyoxyl stearate.

Another embodiment comprises from 0.005% to 0.05% cyclosporin A, Clary sage oil, and polyoxyl stearate.

Another embodiment comprises from 0.005% to 0.05% cyclosporin A, Nutmeg oil, and polyoxyl stearate.

Another embodiment comprises from 0.005% to 0.05% cyclosporin A, Tea tree oil, and polyoxyl stearate.

Another embodiment comprises from 0.005% to 0.05% cyclosporin A, an oil having a specific gravity from 0.95 to 1.07, and propylene glycol stearate.

Another embodiment comprises from 0.005% to 0.05% cyclosporin A, Anise oil, and propylene glycol stearate.

Another embodiment comprises from 0.005% to 0.05% cyclosporin A, Castor oil, and propylene glycol stearate.

Another embodiment comprises from 0.005% to 0.05% cyclosporin A, Clove oil, and propylene glycol stearate.

Another embodiment comprises from 0.005% to 0.05% cyclosporin A, Cassia oil, and propylene glycol stearate.

Another embodiment comprises from 0.005% to 0.05% cyclosporin A, Cinnamon oil, and propylene glycol stearate.

Another embodiment comprises from 0.005% to 0.05% cyclosporin A, an oil having a specific gravity from 0.90 to 0.95, and propylene glycol stearate.

Another embodiment comprises from 0.005% to 0.05% cyclosporin A, Almond oil, and propylene glycol stearate.

Another embodiment comprises from 0.005% to 0.05% cyclosporin A, Corn oil, and propylene glycol stearate.

Another embodiment comprises from 0.005% to 0.05% cyclosporin A, Arachis oil, and propylene glycol stearate.

Another embodiment comprises from 0.005% to 0.05% cyclosporin A, Cottonseed oil, and propylene glycol stearate.

Another embodiment comprises from 0.005% to 0.05% cyclosporin A, Safflower oil, and propylene glycol stearate.

Another embodiment comprises from 0.005% to 0.05% cyclosporin A, Maize oil, and propylene glycol stearate.

Another embodiment comprises from 0.005% to 0.05% cyclosporin A, Linseed oil, and propylene glycol stearate.

Another embodiment comprises from 0.005% to 0.05% cyclosporin A, Rapeseed oil, and propylene glycol stearate.

Another embodiment comprises from 0.005% to 0.05% cyclosporin A, Soybean oil, and propylene glycol stearate.

Another embodiment comprises from 0.005% to 0.05% cyclosporin A, Olive oil, and propylene glycol stearate.

Another embodiment comprises from 0.005% to 0.05% cyclosporin A, Caraway oil, and propylene glycol stearate.

Another embodiment comprises from 0.005% to 0.05% cyclosporin A, Rosemary oil, and propylene glycol stearate.

Another embodiment comprises from 0.005% to 0.05% cyclosporin A, Peanut oil, and propylene glycol stearate.

Another embodiment comprises from 0.005% to 0.05% cyclosporin A, Peppermint oil, and propylene glycol stearate.

Another embodiment comprises from 0.005% to 0.05% cyclosporin A, Sunflower oil, and propylene glycol stearate.

Another embodiment comprises from 0.005% to 0.05% cyclosporin A, Eucalyptus oil, and propylene glycol stearate.

Another embodiment comprises from 0.005% to 0.05% cyclosporin A, Sesame oil, and propylene glycol stearate.

Another embodiment comprises from 0.005% to 0.05% cyclosporin A, an oil having a specific gravity below 0.9, and propylene glycol stearate.

Another embodiment comprises from 0.005% to 0.05% cyclosporin A, Mineral oil, and propylene glycol stearate.

Another embodiment comprises from 0.005% to 0.05% cyclosporin A, Coriander oil, and propylene glycol stearate.

Another embodiment comprises from 0.005% to 0.05% cyclosporin A, Lavender oil, and propylene glycol stearate.

Another embodiment comprises from 0.005% to 0.05% cyclosporin A, Citronella oil, and propylene glycol stearate.

Another embodiment comprises from 0.005% to 0.05% cyclosporin A, Juniper oil, and propylene glycol stearate.

Another embodiment comprises from 0.005% to 0.05% cyclosporin A, Lemon oil, and propylene glycol stearate.

Another embodiment comprises from 0.005% to 0.05% cyclosporin A, Orange oil, and propylene glycol stearate.

Another embodiment comprises from 0.005% to 0.05% cyclosporin A, Clary sage oil, and propylene glycol stearate.

Another embodiment comprises from 0.005% to 0.05% cyclosporin A, Nutmeg oil, and propylene glycol stearate.

Another embodiment comprises from 0.005% to 0.05% cyclosporin A, Tea tree oil, and propylene glycol stearate.

Another embodiment comprises from 0.005% to 0.05% cyclosporin A, an oil having a specific gravity from 0.95 to 1.07, and sucrose stearate.

Another embodiment comprises from 0.005% to 0.05% cyclosporin A, Anise oil, and sucrose stearate.

Another embodiment comprises from 0.005% to 0.05% cyclosporin A, Castor oil, and sucrose stearate.

Another embodiment comprises from 0.005% to 0.05% cyclosporin A, Clove oil, and sucrose stearate.

Another embodiment comprises from 0.005% to 0.05% cyclosporin A, Cassia oil, and sucrose stearate.

Another embodiment comprises from 0.005% to 0.05% cyclosporin A, Cinnamon oil, and sucrose stearate.

Another embodiment comprises from 0.005% to 0.05% cyclosporin A, an oil having a specific gravity from 0.90 to 0.95, and sucrose stearate.

Another embodiment comprises from 0.005% to 0.05% cyclosporin A, Almond oil, and sucrose stearate.

Another embodiment comprises from 0.005% to 0.05% cyclosporin A, Corn oil, and sucrose stearate.

Another embodiment comprises from 0.005% to 0.05% cyclosporin A, Arachis oil, and sucrose stearate.

Another embodiment comprises from 0.005% to 0.05% cyclosporin A, Cottonseed oil, and sucrose stearate.

Another embodiment comprises from 0.005% to 0.05% cyclosporin A, Safflower oil, and sucrose stearate.

Another embodiment comprises from 0.005% to 0.05% cyclosporin A, Maize oil, and sucrose stearate.

Another embodiment comprises from 0.005% to 0.05% cyclosporin A, Linseed oil, and sucrose stearate.

Another embodiment comprises from 0.005% to 0.05% cyclosporin A, Rapeseed oil, and sucrose stearate.

Another embodiment comprises from 0.005% to 0.05% cyclosporin A, Soybean oil, and sucrose stearate.

Another embodiment comprises from 0.005% to 0.05% cyclosporin A, Olive oil, and sucrose stearate.

Another embodiment comprises from 0.005% to 0.05% cyclosporin A, Caraway oil, and sucrose stearate.

Another embodiment comprises from 0.005% to 0.05% cyclosporin A, Rosemary oil, and sucrose stearate.

Another embodiment comprises from 0.005% to 0.05% cyclosporin A, Peanut oil, and sucrose stearate.

Another embodiment comprises from 0.005% to 0.05% cyclosporin A, Peppermint oil, and sucrose stearate.

Another embodiment comprises from 0.005% to 0.05% cyclosporin A, Sunflower oil, and sucrose stearate.

Another embodiment comprises from 0.005% to 0.05% cyclosporin A, Eucalyptus oil, and sucrose stearate.

Another embodiment comprises from 0.005% to 0.05% cyclosporin A, Sesame oil, and sucrose stearate.

Another embodiment comprises from 0.005% to 0.05% cyclosporin A, an oil having a specific gravity below 0.9, and sucrose stearate.

Another embodiment comprises from 0.005% to 0.05% cyclosporin A, Mineral oil, and sucrose stearate.

Another embodiment comprises from 0.005% to 0.05% cyclosporin A, Coriander oil, and sucrose stearate.

Another embodiment comprises from 0.005% to 0.05% cyclosporin A, Lavender oil, and sucrose stearate.

Another embodiment comprises from 0.005% to 0.05% cyclosporin A, Citronella oil, and sucrose stearate.

Another embodiment comprises from 0.005% to 0.05% cyclosporin A, Juniper oil, and sucrose stearate.

Another embodiment comprises from 0.005% to 0.05% cyclosporin A, Lemon oil, and sucrose stearate.

Another embodiment comprises from 0.005% to 0.05% cyclosporin A, Orange oil, and sucrose stearate.

Another embodiment comprises from 0.005% to 0.05% cyclosporin A, Clary sage oil, and sucrose stearate.

Another embodiment comprises from 0.005% to 0.05% cyclosporin A, Nutmeg oil, and sucrose stearate.

Another embodiment comprises from 0.005% to 0.05% cyclosporin A, Tea tree oil, and sucrose stearate.

Another embodiment comprises from 0.005% to 0.05% cyclosporin A, an oil having a specific gravity from 0.95 to 1.07, and polyethylene glycol.

Another embodiment comprises from 0.005% to 0.05% cyclosporin A, Anise oil, and polyethylene glycol.

Another embodiment comprises from 0.005% to 0.05% cyclosporin A, Castor oil, and polyethylene glycol.

Another embodiment comprises from 0.005% to 0.05% cyclosporin A, Clove oil, and polyethylene glycol.

Another embodiment comprises from 0.005% to 0.05% cyclosporin A, Cassia oil, and polyethylene glycol.

Another embodiment comprises from 0.005% to 0.05% cyclosporin A, Cinnamon oil, and polyethylene glycol.

Another embodiment comprises from 0.005% to 0.05% cyclosporin A, an oil having a specific gravity from 0.90 to 0.95, and polyethylene glycol.

Another embodiment comprises from 0.005% to 0.05% cyclosporin A, Almond oil, and polyethylene glycol.

Another embodiment comprises from 0.005% to 0.05% cyclosporin A, Corn oil, and polyethylene glycol.

Another embodiment comprises from 0.005% to 0.05% cyclosporin A, Arachis oil, and polyethylene glycol.

Another embodiment comprises from 0.005% to 0.05% cyclosporin A, Cottonseed oil, and polyethylene glycol.

Another embodiment comprises from 0.005% to 0.05% cyclosporin A, Safflower oil, and polyethylene glycol.

Another embodiment comprises from 0.005% to 0.05% cyclosporin A, Maize oil, and polyethylene glycol.

Another embodiment comprises from 0.005% to 0.05% cyclosporin A, Linseed oil, and polyethylene glycol.

Another embodiment comprises from 0.005% to 0.05% cyclosporin A, Rapeseed oil, and polyethylene glycol.

Another embodiment comprises from 0.005% to 0.05% cyclosporin A, Soybean oil, and polyethylene glycol.

Another embodiment comprises from 0.005% to 0.05% cyclosporin A, Olive oil, and polyethylene glycol.

Another embodiment comprises from 0.005% to 0.05% cyclosporin A, Caraway oil, and polyethylene glycol.

Another embodiment comprises from 0.005% to 0.05% cyclosporin A, Rosemary oil, and polyethylene glycol.

Another embodiment comprises from 0.005% to 0.05% cyclosporin A, Peanut oil, and polyethylene glycol.

Another embodiment comprises from 0.005% to 0.05% cyclosporin A, Peppermint oil, and polyethylene glycol.

Another embodiment comprises from 0.005% to 0.05% cyclosporin A, Sunflower oil, and polyethylene glycol.

Another embodiment comprises from 0.005% to 0.05% cyclosporin A, Eucalyptus oil, and polyethylene glycol.

Another embodiment comprises from 0.005% to 0.05% cyclosporin A, Sesame oil, and polyethylene glycol.

Another embodiment comprises from 0.005% to 0.05% cyclosporin A, an oil having a specific gravity below 0.9, and polyethylene glycol.

Another embodiment comprises from 0.005% to 0.05% cyclosporin A, Mineral oil, and polyethylene glycol.

Another embodiment comprises from 0.005% to 0.05% cyclosporin A, Coriander oil, and polyethylene glycol.

Another embodiment comprises from 0.005% to 0.05% cyclosporin A, Lavender oil, and polyethylene glycol.

Another embodiment comprises from 0.005% to 0.05% cyclosporin A, Citronella oil, and polyethylene glycol.

Another embodiment comprises from 0.005% to 0.05% cyclosporin A, Juniper oil, and polyethylene glycol.

Another embodiment comprises from 0.005% to 0.05% cyclosporin A, Lemon oil, and polyethylene glycol.

Another embodiment comprises from 0.005% to 0.05% cyclosporin A, Orange oil, and polyethylene glycol.

Another embodiment comprises from 0.005% to 0.05% cyclosporin A, Clary sage oil, and polyethylene glycol.

Another embodiment comprises from 0.005% to 0.05% cyclosporin A, Nutmeg oil, and polyethylene glycol.

Another embodiment comprises from 0.005% to 0.05% cyclosporin A, Tea tree oil, and polyethylene glycol.

Another embodiment comprises from 0.005% to 0.05% cyclosporin A, an oil having a specific gravity from 0.95 to 1.07, and polyethylene oxide.

Another embodiment comprises from 0.005% to 0.05% cyclosporin A, Anise oil, and polyethylene oxide.

Another embodiment comprises from 0.005% to 0.05% cyclosporin A, Castor oil, and polyethylene oxide.

Another embodiment comprises from 0.005% to 0.05% cyclosporin A, Clove oil, and polyethylene oxide.

Another embodiment comprises from 0.005% to 0.05% cyclosporin A, Cassia oil, and polyethylene oxide.

Another embodiment comprises from 0.005% to 0.05% cyclosporin A, Cinnamon oil, and polyethylene oxide.

Another embodiment comprises from 0.005% to 0.05% cyclosporin A, an oil having a specific gravity from 0.90 to 0.95, and polyethylene oxide.

Another embodiment comprises from 0.005% to 0.05% cyclosporin A, Almond oil, and polyethylene oxide.

Another embodiment comprises from 0.005% to 0.05% cyclosporin A, Corn oil, and polyethylene oxide.

Another embodiment comprises from 0.005% to 0.05% cyclosporin A, Arachis oil, and polyethylene oxide.

Another embodiment comprises from 0.005% to 0.05% cyclosporin A, Cottonseed oil, and polyethylene oxide.

Another embodiment comprises from 0.005% to 0.05% cyclosporin A, Safflower oil, and polyethylene oxide.

Another embodiment comprises from 0.005% to 0.05% cyclosporin A, Maize oil, and polyethylene oxide.

Another embodiment comprises from 0.005% to 0.05% cyclosporin A, Linseed oil, and polyethylene oxide.

Another embodiment comprises from 0.005% to 0.05% cyclosporin A, Rapeseed oil, and polyethylene oxide.

Another embodiment comprises from 0.005% to 0.05% cyclosporin A, Soybean oil, and polyethylene oxide.

Another embodiment comprises from 0.005% to 0.05% cyclosporin A, Olive oil, and polyethylene oxide.

Another embodiment comprises from 0.005% to 0.05% cyclosporin A, Caraway oil, and polyethylene oxide.

Another embodiment comprises from 0.005% to 0.05% cyclosporin A, Rosemary oil, and polyethylene oxide.

Another embodiment comprises from 0.005% to 0.05% cyclosporin A, Peanut oil, and polyethylene oxide.

Another embodiment comprises from 0.005% to 0.05% cyclosporin A, Peppermint oil, and polyethylene oxide.

Another embodiment comprises from 0.005% to 0.05% cyclosporin A, Sunflower oil, and polyethylene oxide.

Another embodiment comprises from 0.005% to 0.05% cyclosporin A, Eucalyptus oil, and polyethylene oxide.

Another embodiment comprises from 0.005% to 0.05% cyclosporin A, Sesame oil, and polyethylene oxide.

Another embodiment comprises from 0.005% to 0.05% cyclosporin A, an oil having a specific gravity below 0.9, and polyethylene oxide.

Another embodiment comprises from 0.005% to 0.05% cyclosporin A, Mineral oil, and polyethylene oxide.

Another embodiment comprises from 0.005% to 0.05% cyclosporin A, Coriander oil, and polyethylene oxide.

Another embodiment comprises from 0.005% to 0.05% cyclosporin A, Lavender oil, and polyethylene oxide.

Another embodiment comprises from 0.005% to 0.05% cyclosporin A, Citronella oil, and polyethylene oxide.

Another embodiment comprises from 0.005% to 0.05% cyclosporin A, Juniper oil, and polyethylene oxide.

Another embodiment comprises from 0.005% to 0.05% cyclosporin A, Lemon oil, and polyethylene oxide.

Another embodiment comprises from 0.005% to 0.05% cyclosporin A, Orange oil, and polyethylene oxide.

Another embodiment comprises from 0.005% to 0.05% cyclosporin A, Clary sage oil, and polyethylene oxide.

Another embodiment comprises from 0.005% to 0.05% cyclosporin A, Nutmeg oil, and polyethylene oxide.

Another embodiment comprises from 0.005% to 0.05% cyclosporin A, Tea tree oil, and polyethylene oxide.

Another embodiment comprises from 0.005% to 0.05% cyclosporin A, an oil having a specific gravity from 0.95 to 1.07, and polypropylene oxide.

Another embodiment comprises from 0.005% to 0.05% cyclosporin A, Anise oil, and polypropylene oxide.

Another embodiment comprises from 0.005% to 0.05% cyclosporin A, Castor oil, and polypropylene oxide.

Another embodiment comprises from 0.005% to 0.05% cyclosporin A, Clove oil, and polypropylene oxide.

Another embodiment comprises from 0.005% to 0.05% cyclosporin A, Cassia oil, and polypropylene oxide.

Another embodiment comprises from 0.005% to 0.05% cyclosporin A, Cinnamon oil, and polypropylene oxide.

Another embodiment comprises from 0.005% to 0.05% cyclosporin A, an oil having a specific gravity from 0.90 to 0.95, and polypropylene oxide.

Another embodiment comprises from 0.005% to 0.05% cyclosporin A, Almond oil, and polypropylene oxide.

Another embodiment comprises from 0.005% to 0.05% cyclosporin A, Corn oil, and polypropylene oxide.

Another embodiment comprises from 0.005% to 0.05% cyclosporin A, Arachis oil, and polypropylene oxide.

Another embodiment comprises from 0.005% to 0.05% cyclosporin A, Cottonseed oil, and polypropylene oxide.

Another embodiment comprises from 0.005% to 0.05% cyclosporin A, Safflower oil, and polypropylene oxide.

Another embodiment comprises from 0.005% to 0.05% cyclosporin A, Maize oil, and polypropylene oxide.

Another embodiment comprises from 0.005% to 0.05% cyclosporin A, Linseed oil, and polypropylene oxide.

Another embodiment comprises from 0.005% to 0.05% cyclosporin A, Rapeseed oil, and polypropylene oxide.

Another embodiment comprises from 0.005% to 0.05% cyclosporin A, Soybean oil, and polypropylene oxide.

Another embodiment comprises from 0.005% to 0.05% cyclosporin A, Olive oil, and polypropylene oxide.

Another embodiment comprises from 0.005% to 0.05% cyclosporin A, Caraway oil, and polypropylene oxide.

Another embodiment comprises from 0.005% to 0.05% cyclosporin A, Rosemary oil, and polypropylene oxide.

Another embodiment comprises from 0.005% to 0.05% cyclosporin A, Peanut oil, and polypropylene oxide.

Another embodiment comprises from 0.005% to 0.05% cyclosporin A, Peppermint oil, and polypropylene oxide.

Another embodiment comprises from 0.005% to 0.05% cyclosporin A, Sunflower oil, and polypropylene oxide.

Another embodiment comprises from 0.005% to 0.05% cyclosporin A, Eucalyptus oil, and polypropylene oxide.

Another embodiment comprises from 0.005% to 0.05% cyclosporin A, Sesame oil, and polypropylene oxide.

Another embodiment comprises from 0.005% to 0.05% cyclosporin A, an oil having a specific gravity below 0.9, and polypropylene oxide.

Another embodiment comprises from 0.005% to 0.05% cyclosporin A, Mineral oil, and polypropylene oxide.

Another embodiment comprises from 0.005% to 0.05% cyclosporin A, Coriander oil, and polypropylene oxide.

Another embodiment comprises from 0.005% to 0.05% cyclosporin A, Lavender oil, and polypropylene oxide.

Another embodiment comprises from 0.005% to 0.05% cyclosporin A, Citronella oil, and polypropylene oxide.

Another embodiment comprises from 0.005% to 0.05% cyclosporin A, Juniper oil, and polypropylene oxide.

Another embodiment comprises from 0.005% to 0.05% cyclosporin A, Lemon oil, and polypropylene oxide.

Another embodiment comprises from 0.005% to 0.05% cyclosporin A, Orange oil, and polypropylene oxide.

Another embodiment comprises from 0.005% to 0.05% cyclosporin A, Clary sage oil, and polypropylene oxide.

Another embodiment comprises from 0.005% to 0.05% cyclosporin A, Nutmeg oil, and polypropylene oxide.

Another embodiment comprises from 0.005% to 0.05% cyclosporin A, Tea tree oil, and polypropylene oxide.

Another embodiment comprises from 0.005% to 0.05% cyclosporin A, an oil having a specific gravity from 0.95 to 1.07, and a polyethylene oxide-polypropylene oxide copolymer.

Another embodiment comprises from 0.005% to 0.05% cyclosporin A, Anise oil, and a polyethylene oxide-polypropylene oxide copolymer.

Another embodiment comprises from 0.005% to 0.05% cyclosporin A, Castor oil, and a polyethylene oxide-polypropylene oxide copolymer.

Another embodiment comprises from 0.005% to 0.05% cyclosporin A, Clove oil, and a polyethylene oxide-polypropylene oxide copolymer.

Another embodiment comprises from 0.005% to 0.05% cyclosporin A, Cassia oil, and a polyethylene oxide-polypropylene oxide copolymer.

Another embodiment comprises from 0.005% to 0.05% cyclosporin A, Cinnamon oil, and a polyethylene oxide-polypropylene oxide copolymer.

Another embodiment comprises from 0.005% to 0.05% cyclosporin A, an oil having a specific gravity from 0.90 to 0.95, and a polyethylene oxide-polypropylene oxide copolymer.

Another embodiment comprises from 0.005% to 0.05% cyclosporin A, Almond oil, and a polyethylene oxide-polypropylene oxide copolymer.

Another embodiment comprises from 0.005% to 0.05% cyclosporin A, Corn oil, and a polyethylene oxide-polypropylene oxide copolymer.

Another embodiment comprises from 0.005% to 0.05% cyclosporin A, Arachis oil, and a polyethylene oxide-polypropylene oxide copolymer.

Another embodiment comprises from 0.005% to 0.05% cyclosporin A, Cottonseed oil, and a polyethylene oxide-polypropylene oxide copolymer.

Another embodiment comprises from 0.005% to 0.05% cyclosporin A, Safflower oil, and a polyethylene oxide-polypropylene oxide copolymer.

Another embodiment comprises from 0.005% to 0.05% cyclosporin A, Maize oil, and a polyethylene oxide-polypropylene oxide copolymer.

Another embodiment comprises from 0.005% to 0.05% cyclosporin A, Linseed oil, and a polyethylene oxide-polypropylene oxide copolymer.

Another embodiment comprises from 0.005% to 0.05% cyclosporin A, Rapeseed oil, and a polyethylene oxide-polypropylene oxide copolymer.

Another embodiment comprises from 0.005% to 0.05% cyclosporin A, Soybean oil, and a polyethylene oxide-polypropylene oxide copolymer.

Another embodiment comprises from 0.005% to 0.05% cyclosporin A, Olive oil, and a polyethylene oxide-polypropylene oxide copolymer.

Another embodiment comprises from 0.005% to 0.05% cyclosporin A, Caraway oil, and a polyethylene oxide-polypropylene oxide copolymer.

Another embodiment comprises from 0.005% to 0.05% cyclosporin A, Rosemary oil, and a polyethylene oxide-polypropylene oxide copolymer.

Another embodiment comprises from 0.005% to 0.05% cyclosporin A, Peanut oil, and a polyethylene oxide-polypropylene oxide copolymer.

Another embodiment comprises from 0.005% to 0.05% cyclosporin A, Peppermint oil, and a polyethylene oxide-polypropylene oxide copolymer.

Another embodiment comprises from 0.005% to 0.05% cyclosporin A, Sunflower oil, and a polyethylene oxide-polypropylene oxide copolymer.

Another embodiment comprises from 0.005% to 0.05% cyclosporin A, Eucalyptus oil, and a polyethylene oxide-polypropylene oxide copolymer.

Another embodiment comprises from 0.005% to 0.05% cyclosporin A, Sesame oil, and a polyethylene oxide-polypropylene oxide copolymer.

Another embodiment comprises from 0.005% to 0.05% cyclosporin A, an oil having a specific gravity below 0.9, and a polyethylene oxide-polypropylene oxide copolymer.

Another embodiment comprises from 0.005% to 0.05% cyclosporin A, Mineral oil, and a polyethylene oxide-polypropylene oxide copolymer.

Another embodiment comprises from 0.005% to 0.05% cyclosporin A, Coriander oil, and a polyethylene oxide-polypropylene oxide copolymer.

Another embodiment comprises from 0.005% to 0.05% cyclosporin A, Lavender oil, and a polyethylene oxide-polypropylene oxide copolymer.

Another embodiment comprises from 0.005% to 0.05% cyclosporin A, Citronella oil, and a polyethylene oxide-polypropylene oxide copolymer.

Another embodiment comprises from 0.005% to 0.05% cyclosporin A, Juniper oil, and a polyethylene oxide-polypropylene oxide copolymer.

Another embodiment comprises from 0.005% to 0.05% cyclosporin A, Lemon oil, and a polyethylene oxide-polypropylene oxide copolymer.

Another embodiment comprises from 0.005% to 0.05% cyclosporin A, Orange oil, and a polyethylene oxide-polypropylene oxide copolymer.

Another embodiment comprises from 0.005% to 0.05% cyclosporin A, Clary sage oil, and a polyethylene oxide-polypropylene oxide copolymer.

Another embodiment comprises from 0.005% to 0.05% cyclosporin A, Nutmeg oil, and a polyethylene oxide-polypropylene oxide copolymer.

Another embodiment comprises from 0.005% to 0.05% cyclosporin A, Tea tree oil, and a polyethylene oxide-polypropylene oxide copolymer.

Another embodiment comprises from 0.005% to 0.05% cyclosporin A, an oil having a specific gravity from 0.95 to 1.07, and an alcohol ethoxylate.

Another embodiment comprises from 0.005% to 0.05% cyclosporin A, Anise oil, and an alcohol ethoxylate.

Another embodiment comprises from 0.005% to 0.05% cyclosporin A, Castor oil, and an alcohol ethoxylate.

Another embodiment comprises from 0.005% to 0.05% cyclosporin A, Clove oil, and an alcohol ethoxylate.

Another embodiment comprises from 0.005% to 0.05% cyclosporin A, Cassia oil, and an alcohol ethoxylate.

Another embodiment comprises from 0.005% to 0.05% cyclosporin A, Cinnamon oil, and an alcohol ethoxylate.

Another embodiment comprises from 0.005% to 0.05% cyclosporin A, an oil having a specific gravity from 0.90 to 0.95, and an alcohol ethoxylate.

Another embodiment comprises from 0.005% to 0.05% cyclosporin A, Almond oil, and an alcohol ethoxylate.

Another embodiment comprises from 0.005% to 0.05% cyclosporin A, Corn oil, and an alcohol ethoxylate.

Another embodiment comprises from 0.005% to 0.05% cyclosporin A, Arachis oil, and an alcohol ethoxylate.

Another embodiment comprises from 0.005% to 0.05% cyclosporin A, Cottonseed oil, and an alcohol ethoxylate.

Another embodiment comprises from 0.005% to 0.05% cyclosporin A, Safflower oil, and an alcohol ethoxylate.

Another embodiment comprises from 0.005% to 0.05% cyclosporin A, Maize oil, and an alcohol ethoxylate.

Another embodiment comprises from 0.005% to 0.05% cyclosporin A, Linseed oil, and an alcohol ethoxylate.

Another embodiment comprises from 0.005% to 0.05% cyclosporin A, Rapeseed oil, and an alcohol ethoxylate.

Another embodiment comprises from 0.005% to 0.05% cyclosporin A, Soybean oil, and an alcohol ethoxylate.

Another embodiment comprises from 0.005% to 0.05% cyclosporin A, Olive oil, and an alcohol ethoxylate.

Another embodiment comprises from 0.005% to 0.05% cyclosporin A, Caraway oil, and an alcohol ethoxylate.

Another embodiment comprises from 0.005% to 0.05% cyclosporin A, Rosemary oil, and an alcohol ethoxylate.

Another embodiment comprises from 0.005% to 0.05% cyclosporin A, Peanut oil, and an alcohol ethoxylate.

Another embodiment comprises from 0.005% to 0.05% cyclosporin A, Peppermint oil, and an alcohol ethoxylate.

Another embodiment comprises from 0.005% to 0.05% cyclosporin A, Sunflower oil, and an alcohol ethoxylate.

Another embodiment comprises from 0.005% to 0.05% cyclosporin A, Eucalyptus oil, and an alcohol ethoxylate.

Another embodiment comprises from 0.005% to 0.05% cyclosporin A, Sesame oil, and an alcohol ethoxylate.

Another embodiment comprises from 0.005% to 0.05% cyclosporin A, an oil having a specific gravity below 0.9, and an alcohol ethoxylate.

Another embodiment comprises from 0.005% to 0.05% cyclosporin A, Mineral oil, and an alcohol ethoxylate.

Another embodiment comprises from 0.005% to 0.05% cyclosporin A, Coriander oil, and an alcohol ethoxylate.

Another embodiment comprises from 0.005% to 0.05% cyclosporin A, Lavender oil, and an alcohol ethoxylate.

Another embodiment comprises from 0.005% to 0.05% cyclosporin A, Citronella oil, and an alcohol ethoxylate.

Another embodiment comprises from 0.005% to 0.05% cyclosporin A, Juniper oil, and an alcohol ethoxylate.

Another embodiment comprises from 0.005% to 0.05% cyclosporin A, Lemon oil, and an alcohol ethoxylate.

Another embodiment comprises from 0.005% to 0.05% cyclosporin A, Orange oil, and an alcohol ethoxylate.

Another embodiment comprises from 0.005% to 0.05% cyclosporin A, Clary sage oil, and an alcohol ethoxylate.

Another embodiment comprises from 0.005% to 0.05% cyclosporin A, Nutmeg oil, and an alcohol ethoxylate.

Another embodiment comprises from 0.005% to 0.05% cyclosporin A, Tea tree oil, and an alcohol ethoxylate.

Another embodiment comprises from 0.005% to 0.05% cyclosporin A, an oil having a specific gravity from 0.95 to 1.07, and an alkylphenol ethoxylate.

Another embodiment comprises from 0.005% to 0.05% cyclosporin A, Anise oil, and an alkylphenol ethoxylate.

Another embodiment comprises from 0.005% to 0.05% cyclosporin A, Castor oil, and an alkylphenol ethoxylate.

Another embodiment comprises from 0.005% to 0.05% cyclosporin A, Clove oil, and an alkylphenol ethoxylate.

Another embodiment comprises from 0.005% to 0.05% cyclosporin A, Cassia oil, and an alkylphenol ethoxylate.

Another embodiment comprises from 0.005% to 0.05% cyclosporin A, Cinnamon oil, and an alkylphenol ethoxylate.

Another embodiment comprises from 0.005% to 0.05% cyclosporin A, an oil having a specific gravity from 0.90 to 0.95, and an alkylphenol ethoxylate.

Another embodiment comprises from 0.005% to 0.05% cyclosporin A, Almond oil, and an alkylphenol ethoxylate.

Another embodiment comprises from 0.005% to 0.05% cyclosporin A, Corn oil, and an alkylphenol ethoxylate.

Another embodiment comprises from 0.005% to 0.05% cyclosporin A, Arachis oil, and an alkylphenol ethoxylate.

Another embodiment comprises from 0.005% to 0.05% cyclosporin A, Cottonseed oil, and an alkylphenol ethoxylate.

Another embodiment comprises from 0.005% to 0.05% cyclosporin A, Safflower oil, and an alkylphenol ethoxylate.

Another embodiment comprises from 0.005% to 0.05% cyclosporin A, Maize oil, and an alkylphenol ethoxylate.

Another embodiment comprises from 0.005% to 0.05% cyclosporin A, Linseed oil, and an alkylphenol ethoxylate.

Another embodiment comprises from 0.005% to 0.05% cyclosporin A, Rapeseed oil, and an alkylphenol ethoxylate.

Another embodiment comprises from 0.005% to 0.05% cyclosporin A, Soybean oil, and an alkylphenol ethoxylate.

Another embodiment comprises from 0.005% to 0.05% cyclosporin A, Olive oil, and an alkylphenol ethoxylate.

Another embodiment comprises from 0.005% to 0.05% cyclosporin A, Caraway oil, and an alkylphenol ethoxylate.

Another embodiment comprises from 0.005% to 0.05% cyclosporin A, Rosemary oil, and an alkylphenol ethoxylate.

Another embodiment comprises from 0.005% to 0.05% cyclosporin A, Peanut oil, and an alkylphenol ethoxylate.

Another embodiment comprises from 0.005% to 0.05% cyclosporin A, Peppermint oil, and an alkylphenol ethoxylate.

Another embodiment comprises from 0.005% to 0.05% cyclosporin A, Sunflower oil, and an alkylphenol ethoxylate.

Another embodiment comprises from 0.005% to 0.05% cyclosporin A, Eucalyptus oil, and an alkylphenol ethoxylate.

Another embodiment comprises from 0.005% to 0.05% cyclosporin A, Sesame oil, and an alkylphenol ethoxylate.

Another embodiment comprises from 0.005% to 0.05% cyclosporin A, an oil having a specific gravity below 0.9, and an alkylphenol ethoxylate.

Another embodiment comprises from 0.005% to 0.05% cyclosporin A, Mineral oil, and an alkylphenol ethoxylate.

Another embodiment comprises from 0.005% to 0.05% cyclosporin A, Coriander oil, and an alkylphenol ethoxylate.

Another embodiment comprises from 0.005% to 0.05% cyclosporin A, Lavender oil, and an alkylphenol ethoxylate.

Another embodiment comprises from 0.005% to 0.05% cyclosporin A, Citronella oil, and an alkylphenol ethoxylate.

Another embodiment comprises from 0.005% to 0.05% cyclosporin A, Juniper oil, and an alkylphenol ethoxylate.

Another embodiment comprises from 0.005% to 0.05% cyclosporin A, Lemon oil, and an alkylphenol ethoxylate.

Another embodiment comprises from 0.005% to 0.05% cyclosporin A, Orange oil, and an alkylphenol ethoxylate.

Another embodiment comprises from 0.005% to 0.05% cyclosporin A, Clary sage oil, and an alkylphenol ethoxylate.

Another embodiment comprises from 0.005% to 0.05% cyclosporin A, Nutmeg oil, and an alkylphendl ethoxylate.

Another embodiment comprises from 0.005% to 0.05% cyclosporin A, Tea tree oil, and an alkylphenol ethoxylate.

Another embodiment comprises from 0.005% to 0.05% cyclosporin A, an oil having a specific gravity from 0.95 to 1.07, and an alkyl glycoside.

Another embodiment comprises from 0.005% to 0.05% cyclosporin A, Anise oil, and an alkyl glycoside.

Another embodiment comprises from 0.005% to 0.05% cyclosporin A, Castor oil, and an alkyl glycoside.

Another embodiment comprises from 0.005% to 0.05% cyclosporin A, Clove oil, and an alkyl glycoside.

Another embodiment comprises from 0.005% to 0.05% cyclosporin A, Cassia oil, and an alkyl glycoside.

Another embodiment comprises from 0.005% to 0.05% cyclosporin A, Cinnamon oil, and an alkyl glycoside.

Another embodiment comprises from 0.005% to 0.05% cyclosporin A, an oil having a specific gravity from 0.90 to 0.95, and an alkyl glycoside.

Another embodiment comprises from 0.005% to 0.05% cyclosporin A, Almond oil, and an alkyl glycoside.

Another embodiment comprises from 0.005% to 0.05% cyclosporin A, Corn oil, and an alkyl glycoside.

Another embodiment comprises from 0.005% to 0.05% cyclosporin A, Arachis oil, and an alkyl glycoside.

Another embodiment comprises from 0.005% to 0.05% cyclosporin A, Cottonseed oil, and an alkyl glycoside.

Another embodiment comprises from 0.005% to 0.05% cyclosporin A, Safflower oil, and an alkyl glycoside.

Another embodiment comprises from 0.005% to 0.05% cyclosporin A, Maize oil, and an alkyl glycoside.

Another embodiment comprises from 0.005% to 0.05% cyclosporin A, Linseed oil, and an alkyl glycoside.

Another embodiment comprises from 0.005% to 0.05% cyclosporin A, Rapeseed oil, and an alkyl glycoside.

Another embodiment comprises from 0.005% to 0.05% cyclosporin A, Soybean oil, and an alkyl glycoside.

Another embodiment comprises from 0.005% to 0.05% cyclosporin A, Olive oil, and an alkyl glycoside.

Another embodiment comprises from 0.005% to 0.05% cyclosporin A, Caraway oil, and an alkyl glycoside.

Another embodiment comprises from 0.005% to 0.05% cyclosporin A, Rosemary oil, and an alkyl glycoside.

Another embodiment comprises from 0.005% to 0.05% cyclosporin A, Peanut oil, and an alkyl glycoside.

Another embodiment comprises from 0.005% to 0.05% cyclosporin A, Peppermint oil, and an alkyl glycoside.

Another embodiment comprises from 0.005% to 0.05% cyclosporin A, Sunflower oil, and an alkyl glycoside.

Another embodiment comprises from 0.005% to 0.05% cyclosporin A, Eucalyptus oil, and an alkyl glycoside.

Another embodiment comprises from 0.005% to 0.05% cyclosporin A, Sesame oil, and an alkyl glycoside.

Another embodiment comprises from 0.005% to 0.05% cyclosporin A, an oil having a specific gravity below 0.9, and an alkyl glycoside.

Another embodiment comprises from 0.005% to 0.05% cyclosporin A, Mineral oil, and an alkyl glycoside.

Another embodiment comprises from 0.005% to 0.05% cyclosporin A, Coriander oil, and an alkyl glycoside.

Another embodiment comprises from 0.005% to 0.05% cyclosporin A, Lavender oil, and an alkyl glycoside.

Another embodiment comprises from 0.005% to 0.05% cyclosporin A, Citronella oil, and an alkyl glycoside.

Another embodiment comprises from 0.005% to 0.05% cyclosporin A, Juniper oil, and an alkyl glycoside.

Another embodiment comprises from 0.005% to 0.05% cyclosporin A, Lemon oil, and an alkyl glycoside.

Another embodiment comprises from 0.005% to 0.05% cyclosporin A, Orange oil, and an alkyl glycoside.

Another embodiment comprises from 0.005% to 0.05% cyclosporin A, Clary sage oil, and an alkyl glycoside.

Another embodiment comprises from 0.005% to 0.05% cyclosporin A, Nutmeg oil, and an alkyl glycoside.

Another embodiment comprises from 0.005% to 0.05% cyclosporin A, Tea tree oil, and an alkyl glycoside.

Another embodiment comprises from 0.005% to 0.05% cyclosporin A, an oil having a specific gravity from 0.95 to 1.07, and an alkyl polyglycoside.

Another embodiment comprises from 0.005% to 0.05% cyclosporin A, Anise oil, and an alkyl polyglycoside.

Another embodiment comprises from 0.005% to 0.05% cyclosporin A, Castor oil, and an alkyl polyglycoside.

Another embodiment comprises from 0.005% to 0.05% cyclosporin A, Clove oil, and an alkyl polyglycoside.

Another embodiment comprises from 0.005% to 0.05% cyclosporin A, Cassia oil, and an alkyl polyglycoside.

Another embodiment comprises from 0.005% to 0.05% cyclosporin A, Cinnamon oil, and an alkyl polyglycoside.

Another embodiment comprises from 0.005% to 0.05% cyclosporin A, an oil having a specific gravity from 0.90 to 0.95, and an alkyl polyglycoside.

Another embodiment comprises from 0.005% to 0.05% cyclosporin A, Almond oil, and an alkyl polyglycoside.

Another embodiment comprises from 0.005% to 0.05% cyclosporin A, Corn oil, and an alkyl polyglycoside.

Another embodiment comprises from 0.005% to 0.05% cyclosporin A, Arachis oil, and an alkyl polyglycoside.

Another embodiment comprises from 0.005% to 0.05% cyclosporin A, Cottonseed oil, and an alkyl polyglycoside.

Another embodiment comprises from 0.005% to 0.05% cyclosporin A, Safflower oil, and an alkyl polyglycoside.

Another embodiment comprises from 0.005% to 0.05% cyclosporin A, Maize oil, and an alkyl polyglycoside.

Another embodiment comprises from 0.005% to 0.05% cyclosporin A, Linseed oil, and an alkyl polyglycoside.

Another embodiment comprises from 0.005% to 0.05% cyclosporin A, Rapeseed oil, and an alkyl polyglycoside.

Another embodiment comprises from 0.005% to 0.05% cyclosporin A, Soybean oil, and an alkyl polyglycoside.

Another embodiment comprises from 0.005% to 0.05% cyclosporin A, Olive oil, and an alkyl polyglycoside.

Another embodiment comprises from 0.005% to 0.05% cyclosporin A, Caraway oil, and an alkyl polyglycoside.

Another embodiment comprises from 0.005% to 0.05% cyclosporin A, Rosemary oil, and an alkyl polyglycoside.

Another embodiment comprises from 0.005% to 0.05% cyclosporin A, Peanut oil, and an alkyl polyglycoside.

Another embodiment comprises from 0.005% to 0.05% cyclosporin A, Peppermint oil, and an alkyl polyglycoside.

Another embodiment comprises from 0.005% to 0.05% cyclosporin A, Sunflower oil, and an alkyl polyglycoside.

Another embodiment comprises from 0.005% to 0.05% cyclosporin A, Eucalyptus oil, and an alkyl polyglycoside.

Another embodiment comprises from 0.005% to 0.05% cyclosporin A, Sesame oil, and an alkyl polyglycoside.

Another embodiment comprises from 0.005% to 0.05% cyclosporin A, an oil having a specific gravity below 0.9, and an alkyl polyglycoside.

Another embodiment comprises from 0.005% to 0.05% cyclosporin A, Mineral oil, and an alkyl polyglycoside.

Another embodiment comprises from 0.005% to 0.05% cyclosporin A, Coriander oil, and an alkyl polyglycoside.

Another embodiment comprises from 0.005% to 0.05% cyclosporin A, Lavender oil, and an alkyl polyglycoside.

Another embodiment comprises from 0.005% to 0.05% cyclosporin A, Citronella oil, and an alkyl polyglycoside.

Another embodiment comprises from 0.005% to 0.05% cyclosporin A, Juniper oil, and an alkyl polyglycoside.

Another embodiment comprises from 0.005% to 0.05% cyclosporin A, Lemon oil, and an alkyl polyglycoside.

Another embodiment comprises from 0.005% to 0.05% cyclosporin A, Orange oil, and an alkyl polyglycoside.

Another embodiment comprises from 0.005% to 0.05% cyclosporin A, Clary sage oil, and an alkyl polyglycoside.

Another embodiment comprises from 0.005% to 0.05% cyclosporin A, Nutmeg oil, and an alkyl polyglycoside.

Another embodiment comprises from 0.005% to 0.05% cyclosporin A, Tea tree oil, and an alkyl polyglycoside.

Another embodiment comprises from 0.005% to 0.05% cyclosporin A, an oil having a specific gravity from 0.95 to 1.07, and a fatty alcohol.

Another embodiment comprises from 0.005% to 0.05% cyclosporin A, Anise oil, and a fatty alcohol.

Another embodiment comprises from 0.005% to 0.05% cyclosporin A, Castor oil, and a fatty alcohol.

Another embodiment comprises from 0.005% to 0.05% cyclosporin A, Clove oil, and a fatty alcohol.

Another embodiment comprises from 0.005% to 0.05% cyclosporin A, Cassia oil, and a fatty alcohol.

Another embodiment comprises from 0.005% to 0.05% cyclosporin A, Cinnamon oil, and a fatty alcohol.

Another embodiment comprises from 0.005% to 0.05% cyclosporin A, an oil having a specific gravity from 0.90 to 0.95, and a fatty alcohol.

Another embodiment comprises from 0.005% to 0.05% cyclosporin A, Almond oil, and a fatty alcohol.

Another embodiment comprises from 0.005% to 0.05% cyclosporin A, Corn oil, and a fatty alcohol.

Another embodiment comprises from 0.005% to 0.05% cyclosporin A, Arachis oil, and a fatty alcohol.

Another embodiment comprises from 0.005% to 0.05% cyclosporin A, Cottonseed oil, and a fatty alcohol.

Another embodiment comprises from 0.005% to 0.05% cyclosporin A, Safflower oil, and a fatty alcohol.

Another embodiment comprises from 0.005% to 0.05% cyclosporin A, Maize oil, and a fatty alcohol.

Another embodiment comprises from 0.005% to 0.05% cyclosporin A, Linseed oil, and a fatty alcohol.

Another embodiment comprises from 0.005% to 0.05% cyclosporin A, Rapeseed oil, and a fatty alcohol.

Another embodiment comprises from 0.005% to 0.05% cyclosporin A, Soybean oil, and a fatty alcohol.

Another embodiment comprises from 0.005% to 0.05% cyclosporin A, Olive oil, and a fatty alcohol.

Another embodiment comprises from 0.005% to 0.05% cyclosporin A, Caraway oil, and a fatty alcohol.

Another embodiment comprises from 0.005% to 0.05% cyclosporin A, Rosemary oil, and a fatty alcohol.

Another embodiment comprises from 0.005% to 0.05% cyclosporin A, Peanut oil, and a fatty alcohol.

Another embodiment comprises from 0.005% to 0.05% cyclosporin A, Peppermint oil, and a fatty alcohol.

Another embodiment comprises from 0.005% to 0.05% cyclosporin A, Sunflower oil, and a fatty alcohol.

Another embodiment comprises from 0.005% to 0.05% cyclosporin A, Eucalyptus oil, and a fatty alcohol.

Another embodiment comprises from 0.005% to 0.05% cyclosporin A, Sesame oil, and a fatty alcohol.

Another embodiment comprises from 0.005% to 0.05% cyclosporin A, an oil having a specific gravity below 0.9, and a fatty alcohol.

Another embodiment comprises from 0.005% to 0.05% cyclosporin A, Mineral oil, and a fatty alcohol.

Another embodiment comprises from 0.005% to 0.05% cyclosporin A, Coriander oil, and a fatty alcohol.

Another embodiment comprises from 0.005% to 0.05% cyclosporin A, Lavender oil, and a fatty alcohol.

Another embodiment comprises from 0.005% to 0.05% cyclosporin A, Citronella oil, and a fatty alcohol.

Another embodiment comprises from 0.005% to 0.05% cyclosporin A, Juniper oil, and a fatty alcohol.

Another embodiment comprises from 0.005% to 0.05% cyclosporin A, Lemon oil, and a fatty alcohol.

Another embodiment comprises from 0.005% to 0.05% cyclosporin A, Orange oil, and a fatty alcohol.

Another embodiment comprises from 0.005% to 0.05% cyclosporin A, Clary sage oil, and a fatty alcohol.

Another embodiment comprises from 0.005% to 0.05% cyclosporin A, Nutmeg oil, and a fatty alcohol.

Another embodiment comprises from 0.005% to 0.05% cyclosporin A, Tea tree oil, and a fatty alcohol.

Another embodiment comprises from 0.005% to 0.05% cyclosporin A, an oil having a specific gravity from 0.95 to 1.07, and a cellulose derivative.

Another embodiment comprises from 0.005% to 0.05% cyclosporin A, Anise oil, and a cellulose derivative.

Another embodiment comprises from 0.005% to 0.05% cyclosporin A, Castor oil, and a cellulose derivative.

Another embodiment comprises from 0.005% to 0.05% cyclosporin A, Clove oil, and a cellulose derivative.

Another embodiment comprises from 0.005% to 0.05% cyclosporin A, Cassia oil, and a cellulose derivative.

Another embodiment comprises from 0.005% to 0.05% cyclosporin A, Cinnamon oil, and a cellulose derivative.

Another embodiment comprises from 0.005% to 0.05% cyclosporin A, an oil having a specific gravity from 0.90 to 0.95, and a cellulose derivative.

Another embodiment comprises from 0.005% to 0.05% cyclosporin A, Almond oil, and a cellulose derivative.

Another embodiment comprises from 0.005% to 0.05% cyclosporin A, Corn oil, and a cellulose derivative.

Another embodiment comprises from 0.005% to 0.05% cyclosporin A, Arachis oil, and a cellulose derivative.

Another embodiment comprises from 0.005% to 0.05% cyclosporin A, Cottonseed oil, and a cellulose derivative.

Another embodiment comprises from 0.005% to 0.05% cyclosporin A, Safflower oil, and a cellulose derivative.

Another embodiment comprises from 0.005% to 0.05% cyclosporin A, Maize oil, and a cellulose derivative.

Another embodiment comprises from 0.005% to 0.05% cyclosporin A, Linseed oil, and a cellulose derivative.

Another embodiment comprises from 0.005% to 0.05% cyclosporin A, Rapeseed oil, and a cellulose derivative.

Another embodiment comprises from 0.005% to 0.05% cyclosporin A, Soybean oil, and a cellulose derivative.

Another embodiment comprises from 0.005% to 0.05% cyclosporin A, Olive oil, and a cellulose derivative.

Another embodiment comprises from 0.005% to 0.05% cyclosporin A, Caraway oil, and a cellulose derivative.

Another embodiment comprises from 0.005% to 0.05% cyclosporin A, Rosemary oil, and a cellulose derivative.

Another embodiment comprises from 0.005% to 0.05% cyclosporin A, Peanut oil, and a cellulose derivative.

Another embodiment comprises from 0.005% to 0.05% cyclosporin A, Peppermint oil, and a cellulose derivative.

Another embodiment comprises from 0.005% to 0.05% cyclosporin A, Sunflower oil, and a cellulose derivative.

Another embodiment comprises from 0.005% to 0.05% cyclosporin A, Eucalyptus oil, and a cellulose derivative.

Another embodiment comprises from 0.005% to 0.05% cyclosporin A, Sesame oil, and a cellulose derivative.

Another embodiment comprises from 0.005% to 0.05% cyclosporin A, an oil having a specific gravity below 0.9, and a cellulose derivative.

Another embodiment comprises from 0.005% to 0.05% cyclosporin A, Mineral oil, and a cellulose derivative.

Another embodiment comprises from 0.005% to 0.05% cyclosporin A, Coriander oil, and a cellulose derivative.

Another embodiment comprises from 0.005% to 0.05% cyclosporin A, Lavender oil, and a cellulose derivative.

Another embodiment comprises from 0.005% to 0.05% cyclosporin A, Citronella oil, and a cellulose derivative.

Another embodiment comprises from 0.005% to 0.05% cyclosporin A, Juniper oil, and a cellulose derivative.

Another embodiment comprises from 0.005% to 0.05% cyclosporin A, Lemon oil, and a cellulose derivative.

Another embodiment comprises from 0.005% to 0.05% cyclosporin A, Orange oil, and a cellulose derivative.

Another embodiment comprises from 0.005% to 0.05% cyclosporin A, Clary sage oil, and a cellulose derivative.

Another embodiment comprises from 0.005% to 0.05% cyclosporin A, Nutmeg oil, and a cellulose derivative.

Another embodiment comprises from 0.005% to 0.05% cyclosporin A, Tea tree oil, and a cellulose derivative.

Another embodiment comprises from 0.005% to 0.05% cyclosporin A, an oil having a specific gravity from 0.95 to 1.07, and hydroxypropylmethyl cellulose.

Another embodiment comprises from 0.005% to 0.05% cyclosporin A, Anise oil, and hydroxypropylmethyl cellulose.

Another embodiment comprises from 0.005% to 0.05% cyclosporin A, Castor oil, and hydroxypropylmethyl cellulose.

Another embodiment comprises from 0.005% to 0.05% cyclosporin A, Clove oil, and hydroxypropylmethyl cellulose.

Another embodiment comprises from 0.005% to 0.05% cyclosporin A, Cassia oil, and hydroxypropylmethyl cellulose.

Another embodiment comprises from 0.005% to 0.05% cyclosporin A, Cinnamon oil, and hydroxypropylmethyl cellulose.

Another embodiment comprises from 0.005% to 0.05% cyclosporin A, an oil having a specific gravity from 0.90 to 0.95, and hydroxypropylmethyl cellulose.

Another embodiment comprises from 0.005% to 0.05% cyclosporin A, Almond oil, and hydroxypropylmethyl cellulose.

Another embodiment comprises from 0.005% to 0.05% cyclosporin A, Corn oil, and hydroxypropylmethyl cellulose.

Another embodiment comprises from 0.005% to 0.05% cyclosporin A, Arachis oil, and hydroxypropylmethyl cellulose.

Another embodiment comprises from 0.005% to 0.05% cyclosporin A, Cottonseed oil, and hydroxypropylmethyl cellulose.

Another embodiment comprises from 0.005% to 0.05% cyclosporin A, Safflower oil, and hydroxypropylmethyl cellulose.

Another embodiment comprises from 0.005% to 0.05% cyclosporin A, Maize oil, and hydroxypropylmethyl cellulose.

Another embodiment comprises from 0.005% to 0.05% cyclosporin A, Linseed oil, and hydroxypropylmethyl cellulose.

Another embodiment comprises from 0.005% to 0.05% cyclosporin A, Rapeseed oil, and hydroxypropylmethyl cellulose.

Another embodiment comprises from 0.005% to 0.05% cyclosporin A, Soybean oil, and hydroxypropylmethyl cellulose.

Another embodiment comprises from 0.005% to 0.05% cyclosporin A, Olive oil, and hydroxypropylmethyl cellulose.

Another embodiment comprises from 0.005% to 0.05% cyclosporin A, Caraway oil, and hydroxypropylmethyl cellulose.

Another embodiment comprises from 0.005% to 0.05% cyclosporin A, Rosemary oil, and hydroxypropylmethyl cellulose.

Another embodiment comprises from 0.005% to 0.05% cyclosporin A, Peanut oil, and hydroxypropylmethyl cellulose.

Another embodiment comprises from 0.005% to 0.05% cyclosporin A, Peppermint oil, and hydroxypropylmethyl cellulose.

Another embodiment comprises from 0.005% to 0.05% cyclosporin A, Sunflower oil, and hydroxypropylmethyl cellulose.

Another embodiment comprises from 0.005% to 0.05% cyclosporin A, Eucalyptus oil, and hydroxypropylmethyl cellulose.

Another embodiment comprises from 0.005% to 0.05% cyclosporin A, Sesame oil, and hydroxypropylmethyl cellulose.

Another embodiment comprises from 0.005% to 0.05% cyclosporin A, an oil having a specific gravity below 0.9, and hydroxypropylmethyl cellulose.

Another embodiment comprises from 0.005% to 0.05% cyclosporin A, Mineral oil, and hydroxypropylmethyl cellulose.

Another embodiment comprises from 0.005% to 0.05% cyclosporin A, Coriander oil, and hydroxypropylmethyl cellulose.

Another embodiment comprises from 0.005% to 0.05% cyclosporin A, Lavender oil, and hydroxypropylmethyl cellulose.

Another embodiment comprises from 0.005% to 0.05% cyclosporin A, Citronella oil, and hydroxypropylmethyl cellulose.

Another embodiment comprises from 0.005% to 0.05% cyclosporin A, Juniper oil, and hydroxypropylmethyl cellulose.

Another embodiment comprises from 0.005% to 0.05% cyclosporin A, Lemon oil, and hydroxypropylmethyl cellulose.

Another embodiment comprises from 0.005% to 0.05% cyclosporin A, Orange oil, and hydroxypropylmethyl cellulose.

Another embodiment comprises from 0.005% to 0.05% cyclosporin A, Clary sage oil, and hydroxypropylmethyl cellulose.

Another embodiment comprises from 0.005% to 0.05% cyclosporin A, Nutmeg oil, and hydroxypropylmethyl cellulose.

Another embodiment comprises from 0.005% to 0.05% cyclosporin A, Tea tree oil, and hydroxypropylmethyl cellulose.

Another embodiment comprises from 0.005% to 0.05% cyclosporin A, an oil having a specific gravity from 0.95 to 1.07, and carboxymethyl cellulose.

Another embodiment comprises from 0.005% to 0.05% cyclosporin A, Anise oil, and carboxymethyl cellulose.

Another embodiment comprises from 0.005% to 0.05% cyclosporin A, Castor oil, and carboxymethyl cellulose.

Another embodiment comprises from 0.005% to 0.05% cyclosporin A, Clove oil, and carboxymethyl cellulose.

Another embodiment comprises from 0.005% to 0.05% cyclosporin A, Cassia oil, and carboxymethyl cellulose.

Another embodiment comprises from 0.005% to 0.05% cyclosporin A, Cinnamon oil, and carboxymethyl cellulose.

Another embodiment comprises from 0.005% to 0.05% cyclosporin A, an oil having a specific gravity from 0.90 to 0.95, and carboxymethyl cellulose.

Another embodiment comprises from 0.005% to 0.05% cyclosporin A, Almond oil, and carboxymethyl cellulose.

Another embodiment comprises from 0.005% to 0.05% cyclosporin A, Corn oil, and carboxymethyl cellulose.

Another embodiment comprises from 0.005% to 0.05% cyclosporin A, Arachis oil, and carboxymethyl cellulose.

Another embodiment comprises from 0.005% to 0.05% cyclosporin A, Cottonseed oil, and carboxymethyl cellulose.

Another embodiment comprises from 0.005% to 0.05% cyclosporin A, Safflower oil, and carboxymethyl cellulose.

Another embodiment comprises from 0.005% to 0.05% cyclosporin A, Maize oil, and carboxymethyl cellulose.

Another embodiment comprises from 0.005% to 0.05% cyclosporin A, Linseed oil, and carboxymethyl cellulose.

Another embodiment comprises from 0.005% to 0.05% cyclosporin A, Rapeseed oil, and carboxymethyl cellulose.

Another embodiment comprises from 0.005% to 0.05% cyclosporin A, Soybean oil, and carboxymethyl cellulose.

Another embodiment comprises from 0.005% to 0.05% cyclosporin A, Olive oil, and carboxymethyl cellulose.

Another embodiment comprises from 0.005% to 0.05% cyclosporin A, Caraway oil, and carboxymethyl cellulose.

Another embodiment comprises from 0.005% to 0.05% cyclosporin A, Rosemary oil, and carboxymethyl cellulose.

Another embodiment comprises from 0.005% to 0.05% cyclosporin A, Peanut oil, and carboxymethyl cellulose.

Another embodiment comprises from 0.005% to 0.05% cyclosporin A, Peppermint oil, and carboxymethyl cellulose.

Another embodiment comprises from 0.005% to 0.05% cyclosporin A, Sunflower oil, and carboxymethyl cellulose.

Another embodiment comprises from 0.005% to 0.05% cyclosporin A, Eucalyptus oil, and carboxymethyl cellulose.

Another embodiment comprises from 0.005% to 0.05% cyclosporin A, Sesame oil, and carboxymethyl cellulose.

Another embodiment comprises from 0.005% to 0.05% cyclosporin A, an oil having a specific gravity below 0.9, and carboxymethyl cellulose.

Another embodiment comprises from 0.005% to 0.05% cyclosporin A, Mineral oil, and carboxymethyl cellulose.

Another embodiment comprises from 0.005% to 0.05% cyclosporin A, Coriander oil, and carboxymethyl cellulose.

Another embodiment comprises from 0.005% to 0.05% cyclosporin A, Lavender oil, and carboxymethyl cellulose.

Another embodiment comprises from 0.005% to 0.05% cyclosporin A, Citronella oil, and carboxymethyl cellulose.

Another embodiment comprises from 0.005% to 0.05% cyclosporin A, Juniper oil, and carboxymethyl cellulose.

Another embodiment comprises from 0.005% to 0.05% cyclosporin A, Lemon oil, and carboxymethyl cellulose.

Another embodiment comprises from 0.005% to 0.05% cyclosporin A, Orange oil, and carboxymethyl cellulose.

Another embodiment comprises from 0.005% to 0.05% cyclosporin A, Clary sage oil, and carboxymethyl cellulose.

Another embodiment comprises from 0.005% to 0.05% cyclosporin A, Nutmeg oil, and carboxymethyl cellulose.

Another embodiment comprises from 0.005% to 0.05% cyclosporin A, Tea tree oil, and carboxymethyl cellulose.

Another embodiment comprises from 0.005% to 0.05% cyclosporin A, an oil having a specific gravity from 0.95 to 1.07, and a polyacrylic acid.

Another embodiment comprises from 0.005% to 0.05% cyclosporin A, Anise oil, and a polyacrylic acid.

Another embodiment comprises from 0.005% to 0.05% cyclosporin A, Castor oil, and a polyacrylic acid.

Another embodiment comprises from 0.005% to 0.05% cyclosporin A, Clove oil, and a polyacrylic acid.

Another embodiment comprises from 0.005% to 0.05% cyclosporin A, Cassia oil, and a polyacrylic acid.

Another embodiment comprises from 0.005% to 0.05% cyclosporin A, Cinnamon oil, and a polyacrylic acid.

Another embodiment comprises from 0.005% to 0.05% cyclosporin A, an oil having a specific gravity from 0.90 to 0.95, and a polyacrylic acid.

Another embodiment comprises from 0.005% to 0.05% cyclosporin A, Almond oil, and a polyacrylic acid.

Another embodiment comprises from 0.005% to 0.05% cyclosporin A, Corn oil, and a polyacrylic acid.

Another embodiment comprises from 0.005% to 0.05% cyclosporin A, Arachis oil, and a polyacrylic acid.

Another embodiment comprises from 0.005% to 0.05% cyclosporin A, Cottonseed oil, and a polyacrylic acid.

Another embodiment comprises from 0.005% to 0.05% cyclosporin A, Safflower oil, and a polyacrylic acid.

Another embodiment comprises from 0.005% to 0.05% cyclosporin A, Maize oil, and a polyacrylic acid.

Another embodiment comprises from 0.005% to 0.05% cyclosporin A, Linseed oil, and a polyacrylic acid.

Another embodiment comprises from 0.005% to 0.05% cyclosporin A, Rapeseed oil, and a polyacrylic acid.

Another embodiment comprises from 0.005% to 0.05% cyclosporin A, Soybean oil, and a polyacrylic acid.

Another embodiment comprises from 0.005% to 0.05% cyclosporin A, Olive oil, and a polyacrylic acid.

Another embodiment comprises from 0.005% to 0.05% cyclosporin A, Caraway oil, and a polyacrylic acid.

Another embodiment comprises from 0.005% to 0.05% cyclosporin A, Rosemary oil, and a polyacrylic acid.

Another embodiment comprises from 0.005% to 0.05% cyclosporin A, Peanut oil, and a polyacrylic acid.

Another embodiment comprises from 0.005% to 0.05% cyclosporin A, Peppermint oil, and a polyacrylic acid.

Another embodiment comprises from 0.005% to 0.05% cyclosporin A, Sunflower oil, and a polyacrylic acid.

Another embodiment comprises from 0.005% to 0.05% cyclosporin A, Eucalyptus oil, and a polyacrylic acid.

Another embodiment comprises from 0.005% to 0.05% cyclosporin A, Sesame oil, and a polyacrylic acid.

Another embodiment comprises from 0.005% to 0.05% cyclosporin A, an oil having a specific gravity below 0.9, and a polyacrylic acid.

Another embodiment comprises from 0.005% to 0.05% cyclosporin A, Mineral oil, and a polyacrylic acid.

Another embodiment comprises from 0.005% to 0.05% cyclosporin A, Coriander oil, and a polyacrylic acid.

Another embodiment comprises from 0.005% to 0.05% cyclosporin A, Lavender oil, and a polyacrylic acid.

Another embodiment comprises from 0.005% to 0.05% cyclosporin A, Citronella oil, and a polyacrylic acid.

Another embodiment comprises from 0.005% to 0.05% cyclosporin A, Juniper oil, and a polyacrylic acid.

Another embodiment comprises from 0.005% to 0.05% cyclosporin A, Lemon oil, and a polyacrylic acid.

Another embodiment comprises from 0.005% to 0.05% cyclosporin A, Orange oil, and a polyacrylic acid.

Another embodiment comprises from 0.005% to 0.05% cyclosporin A, Clary sage oil, and a polyacrylic acid.

Another embodiment comprises from 0.005% to 0.05% cyclosporin A, Nutmeg oil, and a polyacrylic acid.

Another embodiment comprises from 0.005% to 0.05% cyclosporin A, Tea tree oil, and a polyacrylic acid.

Another embodiment comprises from 0.005% to 0.05% cyclosporin A, an oil having a specific gravity from 0.95 to 1.07, and a Carbomer.

Another embodiment comprises from 0.005% to 0.05% cyclosporin A, Anise oil, and a Carbomer.

Another embodiment comprises from 0.005% to 0.05% cyclosporin A, Castor oil, and a Carbomer.

Another embodiment comprises from 0.005% to 0.05% cyclosporin A, Clove oil, and a Carbomer.

Another embodiment comprises from 0.005% to 0.05% cyclosporin A, Cassia oil, and a Carbomer.

Another embodiment comprises from 0.005% to 0.05% cyclosporin A, Cinnamon oil, and a Carbomer.

Another embodiment comprises from 0.005% to 0.05% cyclosporin A, an oil having a specific gravity from 0.90 to 0.95, and a Carbomer.

Another embodiment comprises from 0.005% to 0.05% cyclosporin A, Almond oil, and a Carbomer.

Another embodiment comprises from 0.005% to 0.05% cyclosporin A, Corn oil, and a Carbomer.

Another embodiment comprises from 0.005% to 0.05% cyclosporin A, Arachis oil, and a Carbomer.

Another embodiment comprises from 0.005% to 0.05% cyclosporin A, Cottonseed oil, and a Carbomer.

Another embodiment comprises from 0.005% to 0.05% cyclosporin A, Safflower oil, and a Carbomer.

Another embodiment comprises from 0.005% to 0.05% cyclosporin A, Maize oil, and a Carbomer.

Another embodiment comprises from 0.005% to 0.05% cyclosporin A, Linseed oil, and a Carbomer.

Another embodiment comprises from 0.005% to 0.05% cyclosporin A, Rapeseed oil, and a Carbomer.

Another embodiment comprises from 0.005% to 0.05% cyclosporin A, Soybean oil, and a Carbomer.

Another embodiment comprises from 0.005% to 0.05% cyclosporin A, Olive oil, and a Carbomer.

Another embodiment comprises from 0.005% to 0.05% cyclosporin A, Caraway oil, and a Carbomer.

Another embodiment comprises from 0.005% to 0.05% cyclosporin A, Rosemary oil, and a Carbomer.

Another embodiment comprises from 0.005% to 0.05% cyclosporin A, Peanut oil, and a Carbomer.

Another embodiment comprises from 0.005% to 0.05% cyclosporin A, Peppermint oil, and a Carbomer.

Another embodiment comprises from 0.005% to 0.05% cyclosporin A, Sunflower oil, and a Carbomer.

Another embodiment comprises from 0.005% to 0.05% cyclosporin A, Eucalyptus oil, and a Carbomer.

Another embodiment comprises from 0.005% to 0.05% cyclosporin A, Sesame oil, and a Carbomer.

Another embodiment comprises from 0.005% to 0.05% cyclosporin A, an oil having a specific gravity below 0.9, and a Carbomer.

Another embodiment comprises from 0.005% to 0.05% cyclosporin A, Mineral oil, and a Carbomer.

Another embodiment comprises from 0.005% to 0.05% cyclosporin A, Coriander oil, and a Carbomer.

Another embodiment comprises from 0.005% to 0.05% cyclosporin A, Lavender oil, and a Carbomer.

Another embodiment comprises from 0.005% to 0.05% cyclosporin A, Citronella oil, and a Carbomer.

Another embodiment comprises from 0.005% to 0.05% cyclosporin A, Juniper oil, and a Carbomer.

Another embodiment comprises from 0.005% to 0.05% cyclosporin A, Lemon oil, and a Carbomer.

Another embodiment comprises from 0.005% to 0.05% cyclosporin A, Orange oil, and a Carbomer.

Another embodiment comprises from 0.005% to 0.05% cyclosporin A, Clary sage oil, and a Carbomer.

Another embodiment comprises from 0.005% to 0.05% cyclosporin A, Nutmeg oil, and a Carbomer.

Another embodiment comprises from 0.005% to 0.05% cyclosporin A, Tea tree oil, and a Carbomer.

Another embodiment comprises from 0.005% to 0.05% cyclosporin A, an oil having a specific gravity from 0.95 to 1.07, and a phosphalipid.

Another embodiment comprises from 0.005% to 0.05% cyclosporin A, Anise oil, and a phosphalipid.

Another embodiment comprises from 0.005% to 0.05% cyclosp6rin A, Castor oil, and a phosphalipid.

Another embodiment comprises from 0.005% to 0.05% cyclosporin A, Clove oil, and a phosphalipid.

Another embodiment comprises from 0.005% to 0.05% cyclosporin A, Cassia oil, and a phosphalipid.

Another embodiment comprises from 0.005% to 0.05% cyclosporin A, Cinnamon oil, and a phosphalipid.

Another embodiment comprises from 0.005% to 0.05% cyclosporin A, an oil having a specific gravity from 0.90 to 0.95, and a phosphalipid.

Another embodiment comprises from 0.005% to 0.05% cyclosporin A, Almond oil, and a phosphalipid.

Another embodiment comprises from 0.005% to 0.05% cyclosporin A, Corn oil, and a phosphalipid.

Another embodiment comprises from 0.005% to 0.05% cyclosporin A, Arachis oil, and a phosphalipid.

Another embodiment comprises from 0.005% to 0.05% cyclosporin A, Cottonseed oil, and a phosphalipid.

Another embodiment comprises from 0.005% to 0.05% cyclosporin A, Safflower oil, and a phosphalipid.

Another embodiment comprises from 0.005% to 0.05% cyclosporin A, Maize oil, and a phosphalipid.

Another embodiment comprises from 0.005% to 0.05% cyclosporin A, Linseed oil, and a phosphalipid.

Another embodiment comprises from 0.005% to 0.05% cyclosporin A, Rapeseed oil, and a phosphalipid.

Another embodiment comprises from 0.005% to 0.05% cyclosporin A, Soybean oil, and a phosphalipid.

Another embodiment comprises from 0.005% to 0.05% cyclosporin A, Olive oil, and a phosphalipid.

Another embodiment comprises from 0.005% to 0.05% cyclosporin A, Caraway oil, and a phosphalipid.

Another embodiment comprises from 0.005% to 0.05% cyclosporin A, Rosemary oil, and a phosphalipid.

Another embodiment comprises from 0.005% to 0.05% cyclosporin A, Peanut oil, and a phosphalipid.

Another embodiment comprises from 0.005% to 0.05% cyclosporin A, Peppermint oil, and a phosphalipid.

Another embodiment comprises from 0.005% to 0.05% cyclosporin A, Sunflower oil, and a phosphalipid.

Another embodiment comprises from 0.005% to 0.05% cyclosporin A, Eucalyptus oil, and a phosphalipid.

Another embodiment comprises from 0.005% to 0.05% cyclosporin A, Sesame oil, and a phosphalipid.

Another embodiment comprises from 0.005% to 0.05% cyclosporin A, an oil having a specific gravity below 0.9, and a phosphalipid.

Another embodiment comprises from 0.005% to 0.05% cyclosporin A, Mineral oil, and a phosphalipid.

Another embodiment comprises from 0.005% to 0.05% cyclosporin A, Coriander oil, and a phosphalipid.

Another embodiment comprises from 0.005% to 0.05% cyclosporin A, Lavender oil, and a phosphalipid.

Another embodiment comprises from 0.005% to 0.05% cyclosporin A, Citronella oil, and a phosphalipid.

Another embodiment comprises from 0.005% to 0.05% cyclosporin A, Juniper oil, and a phosphalipid.

Another embodiment comprises from 0.005% to 0.05% cyclosporin A, Lemon oil, and a phosphalipid.

Another embodiment comprises from 0.005% to 0.05% cyclosporin A, Orange oil, and a phosphalipid.

Another embodiment comprises from 0.005% to 0.05% cyclosporin A, Clary sage oil, and a phosphalipid.

Another embodiment comprises from 0.005% to 0.05% cyclosporin A, Nutmeg oil, and a phosphalipid.

Another embodiment comprises from 0.005% to 0.05% cyclosporin A, Tea tree oil, and a phosphalipid.

Another embodiment comprises from 0.005% to 0.05% cyclosporin A, an oil having a specific gravity from 0.95 to 1.07, and phosphatidyl chloline.

Another embodiment comprises from 0.005% to 0.05% cyclosporin A, Anise oil, and phosphatidyl chloline.

Another embodiment comprises from 0.005% to 0.05% cyclosporin A, Castor oil, and phosphatidyl chloline.

Another embodiment comprises from 0.005% to 0.05% cyclosporin A, Clove oil, and phosphatidyl chloline.

Another embodiment comprises from 0.005% to 0.05% cyclosporin A, Cassia oil, and phosphatidyl chloline.

Another embodiment comprises from 0.005% to 0.05% cyclosporin A, Cinnamon oil, and phosphatidyl chloline.

Another embodiment comprises from 0.005% to 0.05% cyclosporin A, an oil having a specific gravity from 0.90 to 0.95, and phosphatidyl chloline.

Another embodiment comprises from 0.005% to 0.05% cyclosporin A, Almond oil, and phosphatidyl chloline.

Another embodiment comprises from 0.005% to 0.05% cyclosporin A, Corn oil, and phosphatidyl chloline.

Another embodiment comprises from 0.005% to 0.05% cyclosporin A, Arachis oil, and phosphatidyl chloline.

Another embodiment comprises from 0.005% to 0.05% cyclosporin A, Cottonseed oil, and phosphatidyl chloline.

Another embodiment comprises from 0.005% to 0.05% cyclosporin A, Safflower oil, and phosphatidyl chloline.

Another embodiment comprises from 0.005% to 0.05% cyclosporin A, Maize oil, and phosphatidyl chloline.

Another embodiment comprises from 0.005% to 0.05% cyclosporin A, Linseed oil, and phosphatidyl chloline.

Another embodiment comprises from 0.005% to 0.05% cyclosporin A, Rapeseed oil, and phosphatidyl chloline.

Another embodiment comprises from 0.005% to 0.05% cyclosporin A, Soybean oil, and phosphatidyl chloline.

Another embodiment comprises from 0.005% to 0.05% cyclosporin A, Olive oil, and phosphatidyl chloline.

Another embodiment comprises from 0.005% to 0.05% cyclosporin A, Caraway oil, and phosphatidyl chloline.

Another embodiment comprises from 0.005% to 0.05% cyclosporin A, Rosemary oil, and phosphatidyl chloline.

Another embodiment comprises from 0.005% to 0.05% cyclosporin A, Peanut oil, and phosphatidyl chloline.

Another embodiment comprises from 0.005% to 0.05% cyclosporin A, Peppermint oil, and phosphatidyl chloline.

Another embodiment comprises from 0.005% to 0.05% cyclosporin A, Sunflower oil, and phosphatidyl chloline.

Another embodiment comprises from 0.005% to 0.05% cyclosporin A, Eucalyptus oil, and phosphatidyl chloline.

Another embodiment comprises from 0.005% to 0.05% cyclosporin A, Sesame oil, and phosphatidyl chloline.

Another embodiment comprises from 0.005% to 0.05% cyclosporin A, an oil having a specific gravity below 0.9, and phosphatidyl chloline.

Another embodiment comprises from 0.005% to 0.05% cyclosporin A, Mineral oil, and phosphatidyl chloline.

Another embodiment comprises from 0.005% to 0.05% cyclosporin A, Coriander oil, and phosphatidyl chloline.

Another embodiment comprises from 0.005% to 0.05% cyclosporin A, Lavender oil, and phosphatidyl chloline.

Another embodiment comprises from 0.005% to 0.05% cyclosporin A, Citronella oil, and phosphatidyl chloline.

Another embodiment comprises from 0.005% to 0.05% cyclosporin A, Juniper oil, and phosphatidyl chloline.

Another embodiment comprises from 0.005% to 0.05% cyclosporin A, Lemon oil, and phosphatidyl chioline.

Another embodiment comprises from 0.005% to 0.05% cyclosporin A, Orange oil, and phosphatidyl chloline.

Another embodiment comprises from 0.005% to 0.05% cyclosporin A, Clary sage oil, and phosphatidyl chlorine.

Another embodiment comprises from 0.005% to 0.05% cyclosporin A, Nutmeg oil, and phosphatidyl chloline.

Another embodiment comprises from 0.005% to 0.05% cyclosporin A, Tea tree oil, and phosphatidyl chloline.

Another embodiment comprises from 0.005% to 0.05% cyclosporin A, an oil having a specific gravity from 0.95 to 1.07, and phosphatidyl serine.

Another embodiment comprises from 0.005% to 0.05% cyclosporin A, Anise oil, and phosphatidyl serine.

Another embodiment comprises from 0.005% to 0.05% cyclosporin A, Castor oil, and phosphatidyl serine.

Another embodiment comprises from 0.005% to 0.05% cyclosporin A, Clove oil, and phosphatidyl serine.

Another embodiment comprises from 0.005% to 0.05% cyclosporin A, Cassia oil, and phosphatidyl serine.

Another embodiment comprises from 0.005% to 0.05% cyclosporin A, Cinnamon oil, and phosphatidyl serine.

Another embodiment comprises from 0.005% to 0.05% cyclosporin A, an oil having a specific gravity from 0.90 to 0.95, and phosphatidyl serine.

Another embodiment comprises from 0.005% to 0.05% cyclosporin A, Almond oil, and phosphatidyl serine.

Another embodiment comprises from 0.005% to 0.05% cyclosporin A, Corn oil, and phosphatidyl serine.

Another embodiment comprises from 0.005% to 0.05% cyclosporin A, Arachis oil, and phosphatidyl serine.

Another embodiment comprises from 0.005% to 0.05% cyclosporin A, Cottonseed oil, and phosphatidyl serine.

Another embodiment comprises from 0.005% to 0.05% cyclosporin A, Safflower oil, and phosphatidyl serine.

Another embodiment comprises from 0.005% to 0.05% cyclosporin A, Maize oil, and phosphatidyl serine.

Another embodiment comprises from 0.005% to 0.05% cyclosporin A, Linseed oil, and phosphatidyl serine.

Another embodiment comprises from 0.005% to 0.05% cyclosporin A, Rapeseed oil, and phosphatidyl serine.

Another embodiment comprises from 0.005% to 0.05% cyclosporin A, Soybean oil, and phosphatidyl serine.

Another embodiment comprises from 0.005% to 0.05% cyclosporin A, Olive oil, and phosphatidyl serine.

Another embodiment comprises from 0.005% to 0.05% cyclosporin A, Caraway oil, and phosphatidyl serine.

Another embodiment comprises from 0.005% to 0.05% cyclosporin A, Rosemary oil, and phosphatidyl serine.

Another embodiment comprises from 0.005% to 0.05% cyclosporin A, Peanut oil, and phosphatidyl serine.

Another embodiment comprises from 0.005% to 0.05% cyclosporin A, Peppermint oil, and phosphatidyl serine.

Another embodiment comprises from 0.005% to 0.05% cyclosporin A, Sunflower oil, and phosphatidyl serine.

Another embodiment comprises from 0.005% to 0.05% cyclosporin A, Eucalyptus oil, and phosphatidyl serine.

Another embodiment comprises from 0.005% to 0.05% cyclosporin A, Sesame oil, and phosphatidyl serine.

Another embodiment comprises from 0.005% to 0.05% cyclosporin A, an oil having a specific gravity below 0.9, and phosphatidyl serine.

Another embodiment comprises from 0.005% to 0.05% cyclosporin A, Mineral oil, and phosphatidyl serine.

Another embodiment comprises from 0.005% to 0.05% cyclosporin A, Coriander oil, and phosphatidyl serine.

Another embodiment comprises from 0.005% to 0.05% cyclosporin A, Lavender oil, and phosphatidyl serine.

Another embodiment comprises from 0.005% to 0.05% cyclosporin A, Citronella oil, and phosphatidyl serine.

Another embodiment comprises from 0.005% to 0.05% cyclosporin A, Juniper oil, and phosphatidyl serine.

Another embodiment comprises from 0.005% to 0.05% cyclosporin A, Lemon oil, and phosphatidyl serine.

Another embodiment comprises from 0.005% to 0.05% cyclosporin A, Orange oil, and phosphatidyl serine.

Another embodiment comprises from 0.005% to 0.05% cyclosporin A, Clary sage oil, and phosphatidyl serine.

Another embodiment comprises from 0.005% to 0.05% cyclosporin A, Nutmeg oil, and phosphatidyl serine.

Another embodiment comprises from 0.005% to 0.05% cyclosporin A, Tea tree oil, and phosphatidyl serine.

Another embodiment comprises about 0.05% cyclosporin A, an oil having a specific gravity from 0.95 to 1.07, and a sorbitan ester.

Another embodiment comprises about 0.05% cyclosporin A, Anise oil, and a sorbitan ester.

Another embodiment comprises about 0.05% cyclosporin A, Castor oil, and a sorbitan ester.

Another embodiment comprises about 0.05% cyclosporin A, Clove oil, and a sorbitan ester.

Another embodiment comprises about 0.05% cyclosporin A, Cassia oil, and a sorbitan ester.

Another embodiment comprises about 0.05% cyclosporin A, Cinnamon oil, and a sorbitan ester.

Another embodiment comprises about 0.05% cyclosporin A, an oil having a specific gravity from 0.90 to 0.95, and a sorbitan ester.

Another embodiment comprises about 0.05% cyclosporin A, Almond oil, and a sorbitan ester.

Another embodiment comprises about 0.05% cyclosporin A, Corn oil, and a sorbitan ester.

Another embodiment comprises about 0.05% cyclosporin A, Arachis oil, and a sorbitan ester.

Another embodiment comprises about 0.05% cyclosporin A, Cottonseed oil, and a sorbitan ester.

Another embodiment comprises about 0.05% cyclosporin A, Safflower oil, and a sorbitan ester.

Another embodiment comprises about 0.05% cyclosporin A, Maize oil, and a sorbitan ester.

Another embodiment comprises about 0.05% cyclosporin A, Linseed oil, and a sorbitan ester.

Another embodiment comprises about 0.05% cyclosporin A, Rapeseed oil, and a sorbitan ester.

Another embodiment comprises about 0.05% cyclosporin A, Soybean oil, and a sorbitan ester.

Another embodiment comprises about 0.05% cyclosporin A, Olive oil, and a sorbitan ester.

Another embodiment comprises about 0.05% cyclosporin A, Caraway oil, and a sorbitan ester.

Another embodiment comprises about 0.05% cyclosporin A, Rosemary oil, and a sorbitan ester.

Another embodiment comprises about 0.05% cyclosporin A, Peanut oil, and a sorbitan ester.

Another embodiment comprises about 0.05% cyclosporin A, Peppermint oil, and a sorbitan ester.

Another embodiment comprises about 0.05% cyclosporin A, Sunflower oil, and a sorbitan ester.

Another embodiment comprises about 0.05% cyclosporin A, Eucalyptus oil, and a sorbitan ester.

Another embodiment comprises about 0.05% cyclosporin A, Sesame oil, and a sorbitan ester.

Another embodiment comprises about 0.05% cyclosporin A, an oil having a specific gravity below 0.9, and a sorbitan ester.

Another embodiment comprises about 0.05% cyclosporin A, Mineral oil, and a sorbitan ester.

Another embodiment comprises about 0.05% cyclosporin A, Coriander oil, and a sorbitan ester.

Another embodiment comprises about 0.05% cyclosporin A, Lavender oil, and a sorbitan ester.

Another embodiment comprises about 0.05% cyclosporin A, Citronella oil, and a sorbitan ester.

Another embodiment comprises about 0.05% cyclosporin A, Juniper oil, and a sorbitan ester.

Another embodiment comprises about 0.05% cyclosporin A, Lemon oil, and a sorbitan ester.

Another embodiment comprises about 0.05% cyclosporin A, Orange oil, and a sorbitan ester.

Another embodiment comprises about 0.05% cyclosporin A, Clary sage oil, and a sorbitan ester.

Another embodiment comprises about 0.05% cyclosporin A, Nutmeg oil, and a sorbitan ester.

Another embodiment comprises about 0.05% cyclosporin A, Tea tree oil, and a sorbitan ester.

Another embodiment comprises about 0.05% cyclosporin A, an oil having a specific gravity from 0.95 to 1.07, and Polysorbate 80.

Another embodiment comprises about 0.05% cyclosporin A, Anise oil, and Polysorbate 80.

Another embodiment comprises about 0.05% cyclosporin A, Clove oil, and Polysorbate 80.

Another embodiment comprises about 0.05% cyclosporin A, Cassia oil, and Polysorbate 80.

Another embodiment comprises about 0.05% cyclosporin A, Cinnamon oil, and Polysorbate 80.

Another embodiment comprises about 0.05% cyclosporin A, an oil having a specific gravity from 0.90 to 0.95, and Polysorbate 80.

Another embodiment comprises about 0.05% cyclosporin A, Almond oil, and Polysorbate 80.

Another embodiment comprises about 0.05% cyclosporin A, Corn oil, and Polysorbate 80.

Another embodiment comprises about 0.05% cyclosporin A, Arachis oil, and Polysorbate 80.

Another embodiment comprises about 0.05% cyclosporin A, Cottonseed oil, and Polysorbate 80.

Another embodiment comprises about 0.05% cyclospdrin A, Safflower oil, and Polysorbate 80.

Another embodiment comprises about 0.05% cyclosporin A, Maize oil, and Polysorbate 80.

Another embodiment comprises about 0.05% cyclosporin A, Linseed oil, and Polysorbate 80.

Another embodiment comprises about 0.05% cyclosporin A, Rapeseed oil, and Polysorbate 80.

Another embodiment comprises about 0.05% cyclosporin A, Soybean oil, and Polysorbate 80.

Another embodiment comprises about 0.05% cyclosporin A, Olive oil, and Polysorbate 80.

Another embodiment comprises about 0.05% cyclosporin A, Caraway oil, and Polysorbate 80.

Another embodiment comprises about 0.05% cyclosporin A, Rosemary oil, and Polysorbate 80.

Another embodiment comprises about 0.05% cyclosporin A, Peanut oil, and Polysorbate 80.

Another embodiment comprises about 0.05% cyclosporin A, Peppermint oil, and Polysorbate 80.

Another embodiment comprises about 0.05% cyclosporin A, Sunflower oil, and Polysorbate 80.

Another embodiment comprises about 0.05% cyclosporin A, Eucalyptus oil, and Polysorbate 80.

Another embodiment comprises about 0.05% cyclosporin A, Sesame oil, and Polysorbate 80.

Another embodiment comprises about 0.05% cyclosporin A, an oil having a specific gravity below 0.9, and Polysorbate 80.

Another embodiment comprises about 0.05% cyclosporin A, Mineral oil, and Polysorbate 80.

Another embodiment comprises about 0.05% cyclosporin A, Coriander oil, and Polysorbate 80.

Another embodiment comprises about 0.05% cyclosporin A, Lavender oil, and Polysorbate 80.

Another embodiment comprises about 0.05% cyclosporin A, Citronella oil, and Polysorbate 80.

Another embodiment comprises about 0.05% cyclosporin A, Juniper oil, and Polysorbate 80.

Another embodiment comprises about 0.05% cyclosporin A, Lemon oil, and Polysorbate 80.

Another embodiment comprises about 0.05% cyclosporin A, Orange oil, and Polysorbate 80.

Another embodiment comprises about 0.05% cyclosporin A, Clary sage oil, and Polysorbate 80.

Another embodiment comprises about 0.05% cyclosporin A, Nutmeg oil, and Polysorbate 80.

Another embodiment comprises about 0.05% cyclosporin A, Tea tree oil, and Polysorbate 80.

Another embodiment comprises about 0.05% cyclosporin A, an oil having a specific gravity from 0.95 to 1.07, and Polysorbate 80.

Another embodiment comprises about 0.05% cyclosporin A, Anise oil, and Polysorbate 80.

Another embodiment comprises about 0.05% cyclosporin A, Castor oil, and Polysorbate 80.

Another embodiment comprises about 0.05% cyclosporin A, Clove oil, and Polysorbate 80.

Another embodiment comprises about 0.05% cyclosporin A, Cassia oil, and Polysorbate 80.

Another embodiment comprises about 0.05% cyclosporin A, Cinnamon oil, and Polysorbate 80.

Another embodiment comprises about 0.05% cyclosporin A, an oil having a specific gravity from 0.90 to 0.95, and Polysorbate 80.

Another embodiment comprises about 0.05% cyclosporin A, Almond oil, and Polysorbate 80.

Another embodiment comprises about 0.05% cyclosporin A, Corn oil, and Polysorbate 80.

Another embodiment comprises about 0.05% cyclosporin A, Arachis oil, and Polysorbate 80.

Another embodiment comprises about 0.05% cyclosporin A, Cottonseed oil, and Polysorbate 80.

Another embodiment comprises about 0.05% cyclosporin A, Safflower oil, and Polysorbate 80.

Another embodiment comprises about 0.05% cyclosporin A, Maize oil, and Polysorbate 80.

Another embodiment comprises about 0.05% cyclosporin A, Linseed oil, and Polysorbate 80.

Another embodiment comprises about 0.05% cyclosporin A, Rapeseed oil, and Polysorbate 80.

Another embodiment comprises about 0.05% cyclosporin A, Soybean oil, and Polysorbate 80.

Another embodiment comprises about 0.05% cyclosporin A, Olive oil, and Polysorbate 80.

Another embodiment comprises about 0.05% cyclosporin A, Caraway oil, and Polysorbate 80.

Another embodiment comprises about 0.05% cyclosporin A, Rosemary oil, and Polysorbate 80.

Another embodiment comprises about 0.05% cyclosporin A, Peanut oil, and Polysorbate 80.

Another embodiment comprises about 0.05% cyclosporin A, Peppermint oil, and Polysorbate 80.

Another embodiment comprises about 0.05% cyclosporin A, Sunflower oil, and Polysorbate 80.

Another embodiment comprises about 0.05% cyclosporin A, Eucalyptus oil, and Polysorbate 80.

Another embodiment comprises about 0.05% cyclosporin A, Sesame oil, and Polysorbate 80.

Another embodiment comprises about 0.05% cyclosporin A, an oil having a specific gravity below 0.9, and Polysorbate 80.

Another embodiment comprises about 0.05% cyclosporin A, Mineral oil, and Polysorbate 80.

Another embodiment comprises about 0.05% cyclosporin A, Coriander oil, and Polysorbate 80.

Another embodiment comprises about 0.05% cyclosporin A, Lavender oil, and Polysorbate 80.

Another embodiment comprises about 0.05% cyclosporin A, Citronella oil, and Polysorbate 80.

Another embodiment comprises about 0.05% cyclosporin A, Juniper oil, and Polysorbate 80.

Another embodiment comprises about 0.05% cyclosporin A, Lemon oil, and,Polysorbate 80.

Another embodiment comprises about 0.05% cyclosporin A, Orange oil, and Polysorbate 80.

Another embodiment comprises about 0.05% cyclosporin A, Clary sage oil, and Polysorbate 80.

Another embodiment comprises about 0.05% cyclosporin A, Nutmeg oil, and Polysorbate 80.

Another embodiment comprises about 0.05% cyclosporin A, Tea tree oil, and Polysorbate 80.

Another embodiment comprises about 0.05% cyclosporin A, an oil having a specific gravity from 0.95 to 1.07, and Polysorbate 60.

Another embodiment comprises about 0.05% cyclosporin A, Anise oil, and Polysorbate 60.

Another embodiment comprises about 0.05% cyclosporin A, Castor oil, and Polysorbate 60.

Another embodiment comprises about 0.05% cyclosporin A, Clove oil, and Polysorbate 60.

Another embodiment comprises about 0.05% cyclosporin A, Cassia oil, and Polysorbate 60.

Another embodiment comprises about 0.05% cyclosporin A, Cinnamon oil, and Polysorbate 60.

Another embodiment comprises about 0.05% cyclosporin A, an oil having a specific gravity from 0.90 to 0.95, and Polysorbate 60.

Another embodiment comprises about 0.05% cyclosporin A, Almond oil, and Polysorbate 60.

Another embodiment comprises about 0.05% cyclosporin A, Corn oil, and Polysorbate 60.

Another embodiment comprises about 0.05% cyclosporin A, Arachis oil, and Polysorbate 60.

Another embodiment comprises about 0.05% cyclosporin A, Cottonseed oil, and Polysorbate 60.

Another embodiment comprises about 0.05% cyclosporin A, Safflower oil, and Polysorbate 60.

Another embodiment comprises about 0.05% cyclosporin A, Maize oil, and Polysorbate 60.

Another embodiment comprises about 0.05% cyclosporin A, Linseed oil, and Polysorbate 60.

Another embodiment comprises about 0.05% cyclosporin A, Rapeseed oil, and Polysorbate 60.

Another embodiment comprises about 0.05% cyclosporin A, Soybean oil, and Polysorbate 60.

Another embodiment comprises about 0.05% cyclosporin A, Olive oil, and Polysorbate 60.

Another embodiment comprises about 0.05% cyclosporin A, Caraway oil, and Polysorbate 60.

Another embodiment comprises about 0.05% cyclosporin A, Rosemary oil, and Polysorbate 60.

Another embodiment comprises about 0.05% cyclosporin A, Peanut oil, and Polysorbate 60.

Another embodiment comprises about 0.05% cyclosporin A, Peppermint oil, and Polysorbate 60.

Another embodiment comprises about 0.05% cyclosporin A, Sunflower oil, and Polysorbate 60.

Another embodiment comprises about 0.05% cyclosporin A, Eucalyptus oil, and Polysorbate 60.

Another embodiment comprises about 0.05% cyclosporin A, Sesame oil, and Polysorbate 60.

Another embodiment comprises about 0.05% cyclosporin A, an oil having a specific gravity below 0.9, and Polysorbate 60.

Another embodiment comprises about 0.05% cyclosporin A, Mineral oil, and Polysorbate 60.

Another embodiment comprises about 0.05% cyclosporin A, Coriander oil, and Polysorbate 60.

Another embodiment comprises about 0.05% cyclosporin A, Lavender oil, and Polysorbate 60.

Another embodiment comprises about 0.05% cyclosporin A, Citronella oil, and Polysorbate 60.

Another embodiment comprises about 0.05% cyclosporin A, Juniper oil, and Polysorbate 60.

Another embodiment comprises about 0.05% cyclosporin A, Lemon oil, and Polysorbate 60.

Another embodiment comprises about 0.05% cyclosporin A, Orange oil, and Polysorbate 60.

Another embodiment comprises about 0.05% cyclosporin A, Clary sage oil, and Polysorbate 60.

Another embodiment comprises about 0.05% cyclosporin A, Nutmeg oil, and Polysorbate 60.

Another embodiment comprises about 0.05% cyclosporin A, Tea tree oil, and Polysorbate 60.

Another embodiment comprises about 0.05% cyclosporin A, an oil having a specific gravity from 0.95 to 1.07, and Polysorbate 40.

Another embodiment comprises about 0.05% cyclosporin A, Anise oil, and Polysorbate 40.

Another embodiment comprises about 0.05% cyclosporin A, Castor oil, and Polysorbate 40.

Another embodiment comprises about 0.05% cyclosporin A, Clove oil, and Polysorbate 40.

Another embodiment comprises about 0.05% cyclosporin A, Cassia oil, and Polysorbate 40.

Another embodiment comprises about 0.05% cyclosporin A, Cinnamon oil, and Polysorbate 40.

Another embodiment comprises about 0.05% cyclosporin A, an oil having a specific gravity from 0.90 to 0.95, and Polysorbate 40.

Another embodiment comprises about 0.05% cyclosporin A, Almond oil, and Polysorbate 40.

Another embodiment comprises about 0.05% cyclosporin A, Corn oil, and Polysorbate 40.

Another embodiment comprises about 0.05% cyclosporin A, Arachis oil, and Polysorbate 40.

Another embodiment comprises about 0.05% cyclosporin A, Cottonseed oil, and Polysorbate 40.

Another embodiment comprises about 0.05% cyclosporin A, Safflower oil, and Polysorbate 40.

Another embodiment comprises about 0.05% cyclosporin A, Maize oil, and Polysorbate 40.

Another embodiment comprises about 0.05% cyclosporin A, Linseed oil, and Polysorbate 40.

Another embodiment comprises about 0.05% cyclosporin A, Rapeseed oil, and Polysorbate 40.

Another embodiment comprises about 0.05% cyclosporin A, Soybean oil, and Polysorbate 40.

Another embodiment comprises about 0.05% cyclosporin A, Olive oil, and Polysorbate 40.

Another embodiment comprises about 0.05% cyclosporin A, Caraway oil, and Polysorbate 40.

Another embodiment comprises about 0.05% cyclosporin A, Rosemary oil, and Polysorbate 40.

Another embodiment comprises about 0.05% cyclosporin A, Peanut oil, and Polysorbate 40.

Another embodiment comprises about 0.05% cyclosporin A, Peppermint oil, and Polysorbate 40.

Another embodiment comprises about 0.05% cyclosporin A, Sunflower oil, and Polysorbate 40.

Another embodiment comprises about 0.05% cyclosporin A, Eucalyptus oil, and Polysorbate 40.

Another embodiment comprises about 0.05% cyclosporin A, Sesame oil, and Polysorbate 40.

Another embodiment comprises about 0.05% cyclosporin A, an oil having a specific gravity below 0.9, and Polysorbate 40.

Another embodiment comprises about 0.05% cyclosporin A, Mineral oil, and Polysorbate 40.

Another embodiment comprises about 0.05% cyclosporin A, Coriander oil, and Polysorbate 40.

Another embodiment comprises about 0.05% cyclosporin A, Lavender oil, and Polysorbate 40.

Another embodiment comprises about 0.05% cyclosporin A, Citronella oil, and Polysorbate 40.

Another embodiment comprises about 0.05% cyclosporin A, Juniper oil, and Polysorbate 40.

Another embodiment comprises about 0.05% cyclosporin A, Lemon oil, and Polysorbate 40.

Another embodiment comprises about 0.05% cyclosporin A, Orange oil, and Polysorbate 40.

Another embodiment comprises about 0.05% cyclosporin A, Clary sage oil, and Polysorbate 40.

Another embodiment comprises about 0.05% cyclosporin A, Nutmeg oil, and Polysorbate 40.

Another embodiment comprises about 0.05% cyclosporin A, Tea tree oil, and Polysorbate 40.

Another embodiment comprises about 0.05% cyclosporin A, an oil having a specific gravity from 0.95 to 1.07, and Polysorbate 20.

Another embodiment comprises about 0.05% cyclosporin A, Anise oil, and Polysorbate 20.

Another embodiment comprises about 0.05% cyclosporin A, Castor oil, and Polysorbate 20.

Another embodiment comprises about 0.05% cyclosporin A, Clove oil, and Polysorbate 20.

Another embodiment comprises about 0.05% cyclosporin A, Cassia oil, and Polysorbate 20.

Another embodiment comprises about 0.05% cyclosporin A, Cinnamon oil, and Polysorbate 20.

Another embodiment comprises about 0.05% cyclosporin A, an oil having a specific gravity from 0.90 to 0.95, and Polysorbate 20.

Another embodiment comprises about 0.05% cyclosporin A, Almond oil, and Polysorbate 20.

Another embodiment comprises about 0.05% cyclosporin A, Corn oil, and Polysorbate 20.

Another embodiment comprises about 0.05% cyclosporin A, Arachis oil, and Polysorbate 20.

Another embodiment comprises about 0.05% cyclosporin A, Cottonseed oil, and Polysorbate 20.

Another embodiment comprises about 0.05% cyclosporin A, Safflower oil, and Polysorbate 20.

Another embodiment comprises about 0.05% cyclosporin A, Maize oil, and Polysorbate 20.

Another embodiment comprises about 0.05% cyclosporin A, Linseed oil, and Polysorbate 20.

Another embodiment comprises about 0.05% cyclosporin A, Rapeseed oil, and Polysorbate 20.

Another embodiment comprises about 0.05% cyclosporin A, Soybean oil, and Polysorbate 20.

Another embodiment comprises about 0.05% cyclosporin A, Olive oil, and Polysorbate 20.

Another embodiment comprises about 0.05% cyclosporin A, Caraway oil, and Polysorbate 20.

Another embodiment comprises about 0.05% cyclosporin A, Rosemary oil, and Polysorbate 20.

Another embodiment comprises about 0.05% cyclosporin A, Peanut oil, and Polysorbate 20.

Another embodiment comprises about 0.05% cyclosporin A, Peppermint oil, and Polysorbate 20.

Another embodiment comprises about 0.05% cyclosporin A, Sunflower oil, and Polysorbate 20.

Another embodiment comprises about 0.05% cyclosporin A, Eucalyptus oil, and Polysorbate 20.

Another embodiment comprises about 0.05% cyclosporin A, Sesame oil, and Polysorbate 20.

Another embodiment comprises about 0.05% cyclosporin A, an oil having a specific gravity below 0.9, and Polysorbate 20.

Another embodiment comprises about 0.05% cyclosporin A, Mineral oil, and Polysorbate 20.

Another embodiment comprises about 0.05% cyclosporin A, Coriander oil, and Polysorbate 20.

Another embodiment comprises about 0.05% cyclosporin A, Lavender oil, and Polysorbate 20.

Another embodiment comprises about 0.05% cyclosporin A, Citronella oil, and Polysorbate 20.

Another embodiment comprises about 0.05% cyclosporin A, Juniper oil, and Polysorbate 20.

Another embodiment comprises about 0.05% cyclosporin A, Lemon oil, and Polysorbate 20.

Another embodiment comprises about 0.05% cyclosporin A, Orange oil, and Polysorbate 20.

Another embodiment comprises about 0.05% cyclosporin A, Clary sage oil, and Polysorbate 20.

Another embodiment comprises about 0.05% cyclosporin A, Nutmeg oil, and Polysorbate 20.

Another embodiment comprises about 0.05% cyclosporin A, Tea tree oil, and Polysorbate 20.

Another embodiment comprises about 0.05% cyclosporin A, an oil having a specific gravity from 0.95 to 1.07, and a stearate surfactant.

Another embodiment comprises about 0.05% cyclosporin A, Anise oil, and a stearate surfactant.

Another embodiment comprises about 0.05% cyclosporin A, Castor oil, and a stearate surfactant.

Another embodiment comprises about 0.05% cyclosporin A, Clove oil, and a stearate surfactant.

Another embodiment comprises about 0.05% cyclosporin A, Cassia oil, and a stearate surfactant.

Another embodiment comprises about 0.05% cyclosporin A, Cinnamon oil, and a stearate surfactant.

Another embodiment comprises about 0.05% cyclosporin A, an oil having a specific gravity from 0.90 to 0.95, and a stearate surfactant.

Another embodiment comprises about 0.05% cyclosporin A, Almond oil, and a stearate surfactant.

Another embodiment comprises about 0.05% cyclosporin A, Corn oil, and a stearate surfactant.

Another embodiment comprises about 0.05% cyclosporin A, Arachis oil, and a stearate surfactant.

Another embodiment comprises about 0.05% cyclosporin A, Cottonseed oil, and a stearate surfactant.

Another embodiment comprises about 0.05% cyclosporin A, Safflower oil, and a stearate surfactant.

Another embodiment comprises about 0.05% cyclosporin A, Maize oil, and a stearate surfactant.

Another embodiment comprises about 0.05% cyclosporin A, Linseed oil, and a stearate surfactant.

Another embodiment comprises about 0.05% cyclosporin A, Rapeseed oil, and a stearate surfactant.

Another embodiment comprises about 0.05% cyclosporin A, Soybean oil, and a stearate surfactant.

Another embodiment comprises about 0.05% cyclosporin A, Olive oil, and a stearate surfactant.

Another embodiment comprises about 0.05% cyclosporin A, Caraway oil, and a stearate surfactant.

Another embodiment comprises about 0.05% cyclosporin A, Rosemary oil, and a stearate surfactant.

Another embodiment comprises about 0.05% cyclosporin A, Peanut oil, and a stearate surfactant.

Another embodiment comprises about 0.05% cyclosporin A, Peppermint oil, and a stearate surfactant.

Another embodiment comprises about 0.05% cyclosporin A, Sunflower oil, and a stearate surfactant.

Another embodiment comprises about 0.05% cyclosporin A, Eucalyptus oil, and a stearate surfactant.

Another embodimerft comprises about 0.05% cyclosporin A, Sesame oil, and a stearate surfactant.

Another embodiment comprises about 0.05% cyclosporin A, an oil having a specific gravity below 0.9, and a stearate surfactant.

Another embodiment comprises about 0.05% cyclosporin A, Mineral oil, and a stearate surfactant.

Another embodiment comprises about 0.05% cyclosporin A, Coriander oil, and a stearate surfactant.

Another embodiment comprises about 0.05% cyclosporin A, Lavender oil, and a stearate surfactant.

Another embodiment comprises about 0.05% cyclosporin A, Citronella oil, and a stearate surfactant.

Another embodiment comprises about 0.05% cyclosporin A, Juniper oil, and a stearate surfactant.

Another embodiment comprises about 0.05% cyclosporin A, Lemon oil, and a stearate surfactant.

Another embodiment comprises about 0.05% cyclosporin A, Orange oil, and a stearate surfactant.

Another embodiment comprises about 0.05% cyclosporin A, Clary sage oil, and a stearate surfactant.

Another embodiment comprises about 0.05% cyclosporin A, Nutmeg oil, and a stearate surfactant.

Another embodiment comprises about 0.05% cyclosporin A, Tea tree oil, and a stearate surfactant.

Another embodiment comprises about 0.05% cyclosporin A, an oil having a specific gravity from 0.95 to 1.07, and glyceryl stearate.

Another embodiment comprises about 0.05% cyclosporin A, Anise oil, and glyceryl stearate.

Another embodiment comprises about 0.05% cyclosporin A, Castor oil, and glyceryl stearate.

Another embodiment comprises about 0.05% cyclosporin A, Clove oil, and glyceryl stearate.

Another embodiment comprises about 0.05% cyclosporin A, Cassia oil, and glyceryl stearate.

Another embodiment comprises about 0.05% cyclosporin A, Cinnamon oil, and glyceryl stearate.

Another embodiment comprises about 0.05% cyclosporin A, an oil having a specific gravity from 0.90 to 0.95, and glyceryl stearate.

Another embodiment comprises about 0.05% cyclosporin A, Almond oil, and glyceryl stearate.

Another embodiment comprises about 0.05% cyclosporin A, Corn oil, and glyceryl stearate.

Another embodiment comprises about 0.05% cyclosporin A, Arachis oil, and glyceryl stearate.

Another embodiment comprises about 0.05% cyclosporin A, Cottonseed oil, and glyceryl stearate.

Another embodiment comprises about 0.05% cyclosporin A, Safflower oil, and glyceryl stearate.

Another embodiment comprises about 0.05% cyclosporin A, Maize oil, and glyceryl stearate.

Another embodiment comprises about 0.05% cyclosporin A, Linseed oil, and glyceryl stearate.

Another embodiment comprises about 0.05% cyclosporin A, Rapeseed oil, and glyceryl stearate.

Another embodiment comprises about 0.05% cyclosporin A, Soybean oil, and glyceryl stearate.

Another embodiment comprises about 0.05% cyclosporin A, Olive oil, and glyceryl stearate.

Another embodiment comprises about 0.05% cyclosporin A, Caraway oil, and glyceryl stearate.

Another embodiment comprises about 0.05% cyclosporin A, Rosemary oil, and glyceryl stearate.

Another embodiment comprises about 0.05% cyclosporin A, Peanut oil, and glyceryl stearate.

Another embodiment comprises about 0.05% cyclosporin A, Peppermint oil, and glyceryl stearate.

Another embodiment comprises about 0.05% cyclosporin A, Sunflower oil, and glyceryl stearate.

Another embodiment comprises about 0.05% cyclosporin A, Eucalyptus oil, and glyceryl stearate.

Another embodiment comprises about 0.05% cyclosporin A, Sesame oil, and glyceryl stearate.

Another embodiment comprises about 0.05% cyclosporin A, an oil having a specific gravity below 0.9, and glyceryl stearate.

Another embodiment comprises about 0.05% cyclosporin A, Mineral oil, and glyceryl stearate.

Another embodiment comprises about 0.05% cyclosporin A, Coriander oil, and glyceryl stearate.

Another embodiment comprises about 0.05% cyclosporin A, Lavender oil, and glyceryl stearate.

Another embodiment comprises about 0.05% cyclosporin A, Citronella oil, and glyceryl stearate.

Another embodiment comprises about 0.05% cyclosporin A, Juniper oil, and glyceryl stearate.

Another embodiment comprises about 0.05% cyclosporin A, Lemon oil, and glyceryl stearate.

Another embodiment comprises about 0.05% cyclosporin A, Orange oil, and glyceryl stearate.

Another embodiment comprises about 0.05% cyclosporin A, Clary sage oil, and glyceryl stearate.

Another embodiment comprises about 0.05% cyclosporin A, Nutmeg oil, and glyceryl stearate.

Another embodiment comprises about 0.05% cyclosporin A, Tea tree oil, and glyceryl stearate.

Another embodiment comprises about 0.05% cyclosporin A, an oil having a specific gravity from 0.95 to 1.07, and isopropyl stearate.

Another embodiment comprises about 0.05% cyclosporin A, Anise oil, and isopropyl stearate.

Another embodiment comprises about 0.05% cyclosporin A, Castor oil, and isopropyl stearate.

Another embodiment comprises about 0.05% cyclosporin A, Clove oil, and isopropyl stearate.

Another embodiment comprises about 0.05% cyclosporin A, Cassia oil, and isopropyl stearate.

Another embodiment comprises about 0.05% cyclosporin A, Cinnamon oil, and isopropyl stearate.

Another embodiment comprises about 0.05% cyclosporin A, an oil having a specific gravity from 0.90 to 0.95, and isopropyl stearate.

Another embodiment comprises about 0.05% cyclosporin A, Almond oil, and isopropyl stearate.

Another embodiment comprises about 0.05% cyclosporin A, Corn oil, and isopropyl stearate.

Another embodiment comprises about 0.05% cyclosporin A, Arachis oil, and isopropyl stearate.

Another embodiment comprises about 0.05% cyclosporin A, Cottonseed oil, and isopropyl stearate.

Another embodiment comprises about 0.05% cyclosporin A, Safflower oil, and isopropyl stearate.

Another embodiment comprises about 0.05% cyclosporin A, Maize oil, and isopropyl stearate.

Another embodiment comprises about 0.05% cyclosporin A, Linseed oil, and isopropyl stearate.

Another embodiment comprises about 0.05% cyclosporin A, Rapeseed oil, and isopropyl stearate.

Another embodiment comprises about 0.05% cyclosporin A, Soybean oil, and isopropyl stearate.

Another embodiment comprises about 0.05% cyclosporin A, Olive oil, and isopropyl stearate.

Another embodiment comprises about 0.05% cyclosporin A, Caraway oil, and isopropyl stearate.

Another embodiment comprises about 0.05% cyclosporin A, Rosemary oil, and isopropyl stearate.

Another embodiment comprises about 0.05% cyclosporin A, Peanut oil, and isopropyl stearate.

Another embodiment comprises about 0.05% cyclosporin A, Peppermint oil, and isopropyl stearate.

Another embodiment comprises about 0.05% cyclosporin A, Sunflower oil, and isopropyl stearate.

Another embodiment comprises about 0.05% cyclosporin A, Eucalyptus oil, and isopropyl stearate.

Another embodiment comprises about 0.05% cyclosporin A, Sesame oil, and isopropyl stearate.

Another embodiment comprises about 0.05% cyclosporin A, an oil having a specific gravity below 0.9, and isopropyl stearate.

Another embodiment comprises about 0.05% cyclosporin A, Mineral oil, and isopropyl stearate.

Another embodiment comprises about 0.05% cyclosporin A, Coriander oil, and isopropyl stearate.

Another embodiment comprises about 0.05% cyclosporin A, Lavender oil, and isopropyl stearate.

Another embodiment comprises about 0.05% cyclosporin A, Citronella oil, and isopropyl stearate.

Another embodiment comprises about 0.05% cyclosporin A, Juniper oil, and isopropyl stearate.

Another embodiment comprises about 0.05% cyclosporin A, Lemon oil, and isopropyl stearate.

Another embodiment comprises about 0.05% cyclosporin A, Orange oil, and isopropyl stearate.

Another embodiment comprises about 0.05% cyclosporin A, Clary sage oil, and isopropyl stearate.

Another embodiment comprises about 0.05% cyclosporin A, Nutmeg oil, and isopropyl stearate.

Another embodiment comprises about 0.05% cyclosporin A, Tea tree oil, and isopropyl stearate.

Another embodiment comprises about 0.05% cyclosporin A, an oil having a specific gravity from 0.95 to 1.07, and polyoxyl stearate.

Another embodiment comprises about 0.05% cyclosporin A, Anise oil, and polyoxyl stearate.

Another embodiment comprises about 0.05% cyclosporin A, Castor oil, and polyoxyl stearate.

Another embodiment comprises about 0.05% cyclosporin A, Clove oil, and polyoxyl stearate.

Another embodiment comprises about 0.05% cyclosporin A, Cassia oil, and polyoxyl stearate.

Another embodiment comprises about 0.05% cyclosporin A, Cinnamon oil, and polyoxyl stearate.

Another embodiment comprises about 0.05% cyclosporin A, an oil having a specific gravity from 0.90 to 0.95, and polyoxyl stearate.

Another embodiment comprises about 0.05% cyclosporin A, Almond oil, and polyoxyl stearate.

Another embodiment comprises about 0.05% cyclosporin A, Corn oil, and polyoxyl stearate.

Another embodiment comprises about 0.05% cyclosporin A, Arachis oil, and polyoxyl stearate.

Another embodiment comprises about 0.05% cyclosporin A, Cottonseed oil, and polyoxyl stearate.

Another embodiment comprises about 0.05% cyclosporin A, Safflower oil, and polyoxyl stearate.

Another embodiment comprises about 0.05% cyclosporin A, Maize oil, and polyoxyl stearate.

Another embodiment comprises about 0.05% cyclosporin A, Linseed oil, and polyoxyl stearate.

Another embodiment comprises about 0.05% cyclosporin A, Rapeseed oil, and polyoxyl stearate.

Another embodiment comprises about 0.05% cyclosporin A, Soybean oil, and polyoxyl stearate.

Another embodiment comprises about 0.05% cyclosporin A, Olive oil, and polyoxyl stearate.

Another embodiment comprises about 0.05% cyclosporin A, Caraway oil, and polyoxyl stearate.

Another embodiment comprises about 0.05% cyclosporin A, Rosemary oil, and polyoxyl stearate.

Another embodiment comprises about 0.05% cyclosporin A, Peanut oil, and polyoxyl stearate.

Another embodiment comprises about 0.05% cyclosporin A, Peppermint oil, and polyoxyl stearate.

Another embodiment comprises about 0.05% cyclosporin A, Sunflower oil, and polyoxyl stearate.

Another embodiment comprises about 0.05% cyclosporin A, Eucalyptus oil, and polyoxyl stearate.

Another embodiment comprises about 0.05% cyclosporin A, Sesame oil, and polyoxyl stearate.

Another embodiment comprises about 0.05% cyclosporin A, an oil having a specific gravity below 0.9, and polyoxyl stearate.

Another embodiment comprises about 0.05% cyclosporin.A, Mineral oil, and polyoxyl stearate.

Another embodiment comprises about 0.05% cyclosporin A, Coriander oil, and polyoxyl stearate.

Another embodiment comprises about 0.05% cyclosporin A, Lavender oil, and polyoxyl stearate.

Another embodiment comprises about 0.05% cyclosporin A, Citronella oil, and polyoxyl stearate.

Another embodiment comprises about 0.05% cyclosporin A, Juniper oil, and polyoxyl stearate.

Another embodiment comprises about 0.05% cyclosporin A, Lemon oil, and polyoxyl stearate.

Another embodiment comprises about 0.05% cyclosporin A, Orange oil, and polyoxyl stearate.

Another embodiment comprises about 0.05% cyclosporin A, Clary sage oil, and polyoxyl stearate.

Another embodiment comprises about 0.05% cyclosporin A, Nutmeg oil, and polyoxyl stearate.

Another embodiment comprises about 0.05% cyclosporin A, Tea tree oil, and polyoxyl stearate.

Another embodiment comprises about 0.05% cyclosporin A, an oil having a specific gravity from 0.95 to 1.07, and propylene glycol stearate.

Another embodiment comprises about 0.05% cyclosporin A, Anise oil, and propylene glycol stearate.

Another embodiment comprises about 0.05% cyclosporin A, Castor oil, and propylene glycol stearate.

Another embodiment comprises about 0.05% cyclosporin A, Clove oil, and propylene glycol stearate.

Another embodiment comprises about 0.05% cyclosporin A, Cassia oil, and propylene glycol stearate.

Another embodiment comprises about 0.05% cyclosporin A, Cinnamon oil, and propylene glycol stearate.

Another embodiment comprises about 0.05% cyclosporin A, an oil having a specific gravity from 0.90 to 0.95, and propylene glycol stearate.

Another embodiment comprises about 0.05% cyclosporin A, Almond oil, and propylene glycol stearate.

Another embodiment comprises about 0.05% cyclosporin A, Corn oil, and propylene glycol stearate.

Another embodiment comprises about 0.05% cyclosporin A, Arachis oil, and propylene glycol stearate.

Another embodiment comprises about 0.05% cyclosporin A, Cottonseed oil, and propylene glycol stearate.

Another embodiment comprises about 0.05% cyclosporin A, Safflower oil, and propylene glycol stearate.

Another embodiment comprises about 0.05% cyclosporin A, Maize oil, and propylene glycol stearate.

Another embodiment comprises about 0.05% cyclosporin A, Linseed oil, and propylene glycol stearate.

Another embodiment comprises about 0.05% cyclosporin A, Rapeseed oil, and propylene glycol stearate.

Another embodiment comprises about 0.05% cyclosporin A, Soybean oil, and propylene glycol stearate.

Another embodiment comprises about 0.05% cyclosporin A, Olive oil, and propylene glycol stearate.

Another embodiment comprises about 0.05% cyclosporin A, Caraway oil, and propylene glycol stearate.

Another embodiment comprises about 0.05% cyclosporin A, Rosemary oil, and propylene glycol stearate.

Another embodiment comprises about 0.05% cyclosporin A, Peanut oil, and propylene glycol stearate.

Another embodiment comprises about 0.05% cyclosporin A, Peppermint oil, and propylene glycol stearate.

Another embodiment comprises about 0.05% cyclosporin A, Sunflower oil, and propylene glycol stearate.

Another embodiment comprises about 0.05% cyclosporin A, Eucalyptus oil, and propylene glycol stearate.

Another embodiment comprises about 0.05% cyclosporin A, Sesame oil, and propylene glycol stearate.

Another embodiment comprises about 0.05% cyclosporin A, an oil having a specific gravity below 0.9, and propylene glycol stearate.

Another embodiment comprises about 0.05% cyclosporin A, Mineral oil, and propylene glycol stearate.

Another embodiment comprises about 0.05% cyclosporin A, Coriander oil, and propylene glycol stearate.

Another embodiment comprises about 0.05% cyclosporin A, Lavender oil, and propylene glycol stearate.

Another embodiment comprises about 0.05% cyclosporin A, Citronella oil, and propylene glycol stearate.

Another embodiment comprises about 0.05% cyclosporin A, Juniper oil, and propylene glycol stearate.

Another embodiment comprises about 0.05% cyclosporin A, Lemon oil, and propylene glycol stearate.

Another embodiment comprises about 0.05% cyclosporin A, Orange oil, and propylene glycol stearate.

Another embodiment comprises about 0.05% cyclosporin A, Clary sage oil, and propylene glycol stearate.

Another embodiment comprises about 0.05% cyclosporin A, Nutmeg oil, and propylene glycol stearate.

Another embodiment comprises about 0.05% cyclosporin A, Tea tree oil, and propylene glycol stearate.

Another embodiment comprises about 0.05% cyclosporin A, an oil having a specific gravity from 0.95 to 1.07, and sucrose stearate.

Another embodiment comprises about 0.05% cyclosporin A, Anise oil, and sucrose stearate.

Another embodiment comprises about 0.05% cyclosporin A, Castor oil, and sucrose stearate.

Another embodiment comprises about 0.05% cyclosporin A, Clove oil, and sucrose stearate.

Another embodiment comprises about 0.05% cyclosporin A, Cassia oil, and sucrose stearate.

Another embodiment comprises about 0.05% cyclosporin A, Cinnamon oil, and sucrose stearate.

Another embodiment comprises about 0.05% cyclosporin A, an oil having a specific gravity from 0.90 to 0.95, and sucrose stearate.

Another embodiment comprises about 0.05% cyclosporin A, Almond oil, and sucrose stearate.

Another embodiment comprises about 0.05% cyclosporin A, Corn oil, and sucrose stearate.

Another embodiment comprises about 0.05% cyclosporin A, Arachis oil, and sucrose stearate.

Another embodiment comprises about 0.05% cyclosporin A, Cottonseed oil, and sucrose stearate.

Another embodiment comprises about 0.05% cyclosporin A, Safflower oil, and sucrose stearate.

Another embodiment comprises about 0.05% cyclosporin A, Maize oil, and sucrose stearate.

Another embodiment comprises about 0.05% cyclosporin A, Linseed oil, and sucrose stearate.

Another embodiment comprises about 0.05% cyclosporin A, Rapeseed oil, and sucrose stearate.

Another embodiment comprises about 0.05% cyclosporin A, Soybean oil, and sucrose stearate.

Another embodiment comprises about 0.05% cyclosporin A, Olive oil, and sucrose stearate.

Another embodiment comprises about 0.05% cyclosporin A, Caraway oil, and sucrose stearate.

Another embodiment comprises about 0.05% cyclosporin A, Rosemary oil, and sucrose stearate.

Another embodiment comprises about 0.05% cyclosporin A, Peanut oil, and sucrose stearate.

Another embodiment comprises about 0.05% cyclosporin A, Peppermint oil, and sucrose stearate.

Another embodiment comprises about 0.05% cyclosporin A, Sunflower oil, and sucrose stearate.

Another embodiment comprises about 0.05% cyclosporin A, Eucalyptus oil, and sucrose stearate.

Another embodiment comprises about 0.05% cyclosporin A, Sesame oil, and sucrose stearate.

Another embodiment comprises about 0.05% cyclosporin A, an oil having a specific gravity below 0.9, and sucrose stearate.

Another embodiment comprises about 0.05% cyclosporin A, Mineral oil, and sucrose stearate.

Another embodiment comprises about 0.05% cyclosporin A, Coriander oil, and sucrose stearate.

Another embodiment comprises about 0.05% cyclosporin A, Lavender oil, and sucrose stearate.

Another embodiment comprises about 0.05% cyclosporin A, Citronella oil, and sucrose stearate.

Another embodiment comprises about 0.05% cyclosporin A, Juniper oil, and sucrose stearate.

Another embodiment comprises about 0.05% cyclosporin A, Lemon oil, and sucrose stearate.

Another embodiment comprises about 0.05% cyclosporin A, Orange oil, and sucrose stearate.

Another embodiment comprises about 0.05% cyclosporin A, Clary sage oil, and sucrose stearate.

Another embodiment comprises about 0.05% cyclosporin A, Nutmeg oil, and sucrose stearate.

Another embodiment comprises about 0.05% cyclosporin A, Tea tree oil, and sucrose stearate.

Another embodiment comprises about 0.05% cyclosporin A, an oil having a specific gravity from 0.95 to 1.07, and polyethylene glycol.

Another embodiment comprises about 0.05% cyclosporin A, Anise oil, and polyethylene glycol.

Another embodiment comprises about 0.05% cyclosporin A, Castor oil, and polyethylene glycol.

Another embodiment comprises about 0.05% cyclosporin A, Clove oil, and polyethylene glycol.

Another embodiment comprises about 0.05% cyclosporin A, Cassia oil, and polyethylene glycol.

Another embodiment comprises about 0.05% cyclosporin A, Cinnamon oil, and polyethylene glycol.

Another embodiment comprises about 0.05% cyclosporin A, an oil having a specific gravity from 0.90 to 0.95, and polyethylene glycol.

Another embodiment comprises about 0.05% cyclosporin A, Almond oil, and polyethylene glycol.

Another embodiment comprises about 0.05% cyclosporin A, Corn oil, and polyethylene glycol.

Another embodiment comprises about 0.05% cyclosporin A, Arachis oil, and polyethylene glycol.

Another embodiment comprises about 0.05% cyclosporin A, Cottonseed oil, and polyethylene glycol.

Another embodiment comprises about 0.05% cyclospdrin A, Safflower oil, and polyethylene glycol.

Another embodiment comprises about 0.05% cyclosporin A, Maize oil, and polyethylene glycol.

Another embodiment comprises about 0.05% cyclosporin A, Linseed oil, and polyethylene glycol.

Another embodiment comprises about 0.05% cyclosporin A, Rapeseed oil, and polyethylene glycol.

Another embodiment comprises about 0.05% cyclosporin A, Soybean oil, and polyethylene glycol.

Another embodiment comprises about 0.05% cyclosporin A, Olive oil, and polyethylene glycol.

Another embodiment comprises about 0.05% cyclosporin A, Caraway oil, and polyethylene glycol.

Another embodiment comprises about 0.05% cyclosporin A, Rosemary oil, and polyethylene glycol.

Another embodiment comprises about 0.05% cyclosporin A, Peanut oil, and polyethylene glycol.

Another embodiment comprises about 0.05% cyclosporin A, Peppermint oil, and polyethylene glycol.

Another embodiment comprises about 0.05% cyclosporin A, Sunflower oil, and polyethylene glycol.

Another embodiment comprises about 0.05% cyclosporin A, Eucalyptus oil, and polyethylene glycol.

Another embodiment comprises about 0.05% cyclosporin A, Sesame oil, and polyethylene glycol.

Another embodiment comprises about 0.05% cyclosporin A, an oil having a specific gravity below 0.9, and polyethylene glycol.

Another embodiment comprises about 0.05% cyclosporin A, Mineral oil, and polyethylene glycol.

Another embodiment comprises about 0.05% cyclosporin A, Coriander oil, and polyethylene glycol.

Another embodiment comprises about 0.05% cyclosporin A, Lavender oil, and polyethylene glycol.

Another embodiment comprises about 0.05% cyclosporin A, Citronella oil, and polyethylene glycol.

Another embodiment comprises about 0.05% cyclosporin A, Juniper oil, and polyethylene glycol.

Another embodiment comprises about 0.05% cyclosporin A, Lemon oil, and polyethylene glycol.

Another embodiment comprises about 0.05% cyclosporin A, Orange oil, and polyethylene glycol.

Another embodiment comprises about 0.05% cyclosporin A, Clary sage oil, and polyethylene glycol.

Another embodiment comprises about 0.05% cyclosporin A, Nutmeg oil, and polyethylene glycol.

Another embodiment comprises about 0.05% cyclosporin A, Tea tree oil, and polyethylene glycol.

Another embodiment comprises about 0.05% cyclosporin A, an oil having a specific gravity from 0.95 to 1.07, and polyethylene oxide.

Another embodiment comprises about 0.05% cyclosporin A, Anise oil, and polyethylene oxide.

Another embodiment comprises about 0.05% cyclosporin A, Castor oil, and polyethylene oxide.

Another embodiment comprises about 0.05% cyclosporin A, Clove oil, and polyethylene oxide.

Another embodiment comprises about 0.05% cyclosporin A, Cassia oil, and polyethylene oxide.

Another embodiment comprises about 0.05% cyclosporin A, Cinnamon oil, and polyethylene oxide.

Another embodiment comprises about 0.05% cyclosporin A, an oil having a specific gravity from 0.90 to 0.95, and polyethylene oxide.

Another embodiment comprises about 0.05% cyclosporin A, Almond oil, and polyethylene oxide.

Another embodiment comprises about 0.05% cyclosporin A, Corn oil, and polyethylene oxide.

Another embodiment comprises about 0.05% cyclosporin A, Arachis oil, and polyethylene oxide.

Another embodiment comprises about 0.05% cyclosporin A, Cottonseed oil, and polyethylene oxide.

Another embodiment comprises about 0.05% cyclosporin A, Safflower oil, and polyethylene oxide.

Another embodiment comprises about 0.05% cyclosporin A, Maize oil, and polyethylene oxide.

Another embodiment comprises about 0.05% cyclosporin A, Linseed oil, and polyethylene oxide.

Another embodiment comprises about 0.05% cyclosporin A, Rapeseed oil, and polyethylene oxide.

Another embodiment comprises about 0.05% cyclosporin A, Soybean oil, and polyethylene oxide.

Another embodiment comprises about 0.05% cyclosporin A, Olive oil, and polyethylene oxide.

Another embodiment comprises about 0.05% cyclosporin A, Caraway oil, and polyethylene oxide.

Another embodiment comprises about 0.05% cyclosporin A, Rosemary oil, and polyethylene oxide.

Another embodiment comprises about 0.05% cyclosporin A, Peanut oil, and polyethylene oxide.

Another embodiment comprises about 0.05% cyclosporin A, Peppermint oil, and polyethylene oxide.

Another embodiment comprises about 0.05% cyclosporin A, Sunflower oil, and polyethylene oxide.

Another embodiment comprises about 0.05% cyclosporin A, Eucalyptus oil, and polyethylene oxide.

Another embodiment comprises about 0.05% cyclosporin A, Sesame oil, and polyethylene oxide.

Another embodiment comprises about 0.05% cyclosporin A, an oil having a specific gravity below 0.9, and polyethylene oxide.

Another embodiment comprises about 0.05% cyclosporin A, Mineral oil, and polyethylene oxide.

Another embodiment comprises about 0.05% cyclosporin A, Coriander oil, and polyethylene oxide.

Another embodiment comprises about 0.05% cyclosporin A, Lavender oil, and polyethylene oxide.

Another embodiment comprises about 0.05% cyclosporin A, Citronella oil, and polyethylene oxide.

Another embodiment comprises about 0.05% cyclosporin A, Juniper oil, and polyethylene oxide.

Another embodiment comprises about 0.05% cyclosporin A, Lemon oil, and polyethylene oxide.

Another embodiment comprises about 0.05% cyclosporin A, Orange oil, and polyethylene oxide.

Another embodiment comprises about 0.05% cyclosporin A, Clary sage oil, and polyethylene oxide.

Another embodiment comprises about 0.05% cyclosporin A, Nutmeg oil, and polyethylene oxide.

Another embodiment comprises about 0.05% cyclosporin A, Tea tree oil, and polyethylene oxide.

Another embodiment comprises about 0.05% cyclosporin A, an oil having a specific gravity from 0.95 to 1.07, and polypropylene oxide.

Another embodiment comprises about 0.05% cyclosporin A, Anise oil, and polypropylene oxide.

Another embodiment comprises about 0.05% cyclosporin A, Castor oil, and polypropylene oxide.

Another embodiment comprises about 0.05% cyclosporin A, Clove oil, and polypropylene oxide.

Another embodiment comprises about 0.05% cyclosporin A, Cassia oil, and polypropylene oxide.

Another embodiment comprises about 0.05% cyclosporin A, Cinnamon oil, and polypropylene oxide.

Another embodiment comprises about 0.05% cyclosporin A, an oil having a specific gravity from 0.90 to 0.95, and polypropylene oxide.

Another embodiment comprises about 0.05% cyclosporin A, Almond oil, and polypropylene oxide.

Another embodiment comprises about 0.05% cyclosporin A, Corn oil, and polypropylene oxide.

Another embodiment comprises about 0.05% cyclosporin A, Arachis oil, and polypropylene oxide.

Another embodiment comprises about 0.05% cyclosporin A, Cottonseed oil, and polypropylene oxide.

Another embodiment comprises about 0.05% cyclosporin A, Safflower oil, and polypropylene oxide.

Another embodiment comprises about 0.05% cyclosporin A, Maize oil, and polypropylene oxide.

Another embodiment comprises about 0.05% cyclosporin A, Linseed oil, and polypropylene oxide.

Another embodiment comprises about 0.05% cyclosporin A, Rapeseed oil, and polypropylene oxide.

Another embodiment comprises about 0.05% cyclosporin A, Soybean oil, and polypropylene oxide.

Another embodiment comprises about 0.05% cyclosporin A, Olive oil, and polypropylene oxide.

Another embodiment comprises about 0.05% cyclosporin A, Caraway oil, and polypropylene oxide.

Another embodiment comprises about 0.05% cyclosporin A, Rosemary oil, and polypropylene oxide.

Another embodiment comprises about 0.05% cyclosporin A, Peanut oil, and polypropylene oxide.

Another embodiment comprises about 0.05% cyclosporin A, Peppermint oil, and polypropylene oxide.

Another embodiment comprises about 0.05% cyclosporin A, Sunflower oil, and polypropylene oxide.

Another embodiment comprises about 0.05% cyclosporin A, Eucalyptus oil, and polypropylene oxide.

Another embodiment comprises about 0.05% cyclosporin A, Sesame oil, and polypropylene oxide.

Another embodiment comprises about 0.05% cyclosporin A, an oil having a specific gravity below 0.9, and polypropylene oxide.

Another embodiment comprises about 0.05% cyclosporin A, Mineral oil, and polypropylene oxide.

Another embodiment comprises about 0.05% cyclosporin A, Coriander oil, and polypropylene oxide.

Another embodiment comprises about 0.05% cyclosporin A, Lavender oil, and polypropylene oxide.

Another embodiment comprises about 0.05% cyclosporin A, Citronella oil, and polypropylene oxide.

Another embodiment comprises about 0.05% cyclosporin A, Juniper oil, and polypropylene oxide.

Another embodiment comprises about 0.05% cyclosporin A, Lemon oil, and polypropylene oxide.

Another embodiment comprises about 0.05% cyclosporin A, Orange oil, and polypropylene oxide.

Another embodiment comprises about 0.05% cyclosporin A, Clary sage oil, and polypropylene oxide.

Another embodiment comprises about 0.05% cyclosporin A, Nutmeg oil, and polypropylene oxide.

Another embodiment comprises about 0.05% cyclosporin A, Tea tree oil, and polypropylene oxide.

Another embodiment comprises about 0.05% cyclosporin A, an oil having a specific gravity from 0.95 to 1.07, and a polyethylene oxide-polypropylene oxide copolymer.

Another embodiment comprises about 0.05% cyclosporin A, Anise oil, and a polyethylene oxide-polypropylene oxide copolymer.

Another embodiment comprises about 0.05% cyclosporin A, Castor oil, and a polyethylene oxide-polypropylene oxide copolymer.

Another embodiment comprises about 0.05% cyclosporin A, Clove oil, and a polyethylene oxide-polypropylene oxide copolymer.

Another embodiment comprises about 0.05% cyclosporin A, Cassia oil, and a polyethylene oxide-polypropylene oxide copolymer.

Another embodiment comprises about 0.05% cyclosporin A, Cinnamon oil, and a polyethylene oxide-polypropylene oxide copolymer.

Another embodiment comprises about 0.05% cyclosporin A, an oil having a specific gravity from 0.90 to 0.95, and a polyethylene oxide-polypropylene oxide copolymer.

Another embodiment comprises about 0.05% cyclosporin A, Almond oil, and a polyethylene oxide-polypropylene oxide copolymer.

Another embodiment comprises about 0.05% cyclosporin A, Corn oil, and a polyethylene oxide-polypropylene oxide copolymer.

Another embodiment comprises about 0.05% cyclosporin A, Arachis oil, and a polyethylene oxide-polypropylene oxide copolymer.

Another embodiment comprises about 0.05% cyclosporin A, Cottonseed oil, and a polyethylene oxide-polypropylene oxide copolymer.

Another embodiment comprises about 0.05% cyclosporin A, Safflower oil, and a polyethylene oxide-polypropylene oxide copolymer.

Another embodiment comprises about 0.05% cyclosporin A, Maize oil, and a polyethylene oxide-polypropylene oxide copolymer.

Another embodiment comprises about 0.05% cyclosporin A, Linseed oil, and a polyethylene oxide-polypropylene oxide copolymer.

Another embodiment comprises about 0.05% cyclosporin A, Rapeseed oil, and a polyethylene oxide-polypropylene oxide copolymer.

Another embodiment comprises about 0.05% cyclosporin A, Soybean oil, and a polyethylene oxide-polypropylene oxide copolymer.

Another embodiment comprises about 0.05% cyclosporin A, Olive oil, and a polyethylene oxide-polypropylene oxide copolymer.

Another embodiment comprises about 0.05% cyclosporin A, Caraway oil, and a polyethylene oxide-polypropylene oxide copolymer.

Another embodiment comprises about 0.05% cyclosporin A, Rosemary oil, and a polyethylene oxide-polypropylene oxide copolymer.

Another embodiment comprises about 0.05% cyclosporin A, Peanut oil, and a polyethylene oxide-polypropylene oxide copolymer.

Another embodiment comprises about 0.05% cyclosporin A, Peppermint oil, and a polyethylene oxide-polypropylene oxide copolymer.

Another embodiment comprises about 0.05% cyclosporin A, Sunflower oil, and a polyethylene oxide-polypropylene oxide copolymer.

Another embodiment comprises about 0.05% cyclosporin A, Eucalyptus oil, and a polyethylene oxide-polypropylene oxide copolymer.

Another embodiment comprises about 0.05% cyclosporin A, Sesame oil, and a polyethylene oxide-polypropylene oxide copolymer.

Another embodiment comprises about 0.05% cyclosporin A, an oil having a specific gravity below 0.9, and a polyethylene oxide-polypropylene oxide copolymer.

Another embodiment comprises about 0.05% cyclosporin A, Mineral oil, and a polyethylene oxide-polypropylene oxide copolymer.

Another embodiment comprises about 0.05% cyclosporin A, Coriander oil, and a polyethylene oxide-polypropylene oxide copolymer.

Another embodiment comprises about 0.05% cyclosporin A, Lavender oil, and a polyethylene oxide-polypropylene oxide copolymer.

Another embodiment comprises about 0.05% cyclosporin A, Citronella oil, and a polyethylene oxide-polypropylene oxide copolymer.

Another embodiment comprises about 0.05% cyclosporin A, Juniper oil, and a polyethylene oxide-polypropylene oxide copolymer.

Another embodiment comprises about 0.05% cyclosporin A, Lemon oil, and a polyethylene oxide-polypropylene oxide copolymer.

Another embodiment comprises about 0.05% cyclosporin A, Orange oil, and a polyethylene oxide-polypropylene oxide copolymer.

Another embodiment comprises about 0.05% cyclosporin A, Clary sage oil, and a polyethylene oxide-polypropylene oxide copolymer.

Another embodiment comprises about 0.05% cyclosporin A, Nutmeg oil, and a polyethylene oxide-polypropylene oxide copolymer.

Another embodiment comprises about 0.05% cyclosporin A, Tea tree oil, and a polyethylene oxide-polypropylene oxide copolymer.

Another embodiment comprises about 0.05% cyclosporin A, an oil having a specific gravity from 0.95 to 1.07, and an alcohol ethoxylate.

Another embodiment comprises about 0.05% cyclosporin A, Anise oil, and an alcohol ethoxylate.

Another embodiment comprises about 0.05% cyclosporin A, Castor oil, and an alcohol ethoxylate.

Another embodiment comprises about 0.05% cyclosporin A, Clove oil, and an alcohol ethoxylate.

Another embodiment comprises about 0.05% cyclosporin A, Cassia oil, and an alcohol ethoxylate.

Another embodiment comprises about 0.05% cyclosporin A, Cinnamon oil, and an alcohol ethoxylate.

Another embodiment comprises about 0.05% cyclosporin A, an oil having a specific gravity from 0.90 to 0.95, and an alcohol ethoxylate.

Another embodiment comprises about 0.05% cyclosporin A, Almond oil, and an alcohol ethoxylate.

Another embodiment comprises about 0.05% cyclosporin A, Corn oil, and an alcohol ethoxylate.

Another embodiment comprises about 0.05% cyclosporin A, Arachis oil, and an alcohol ethoxylate.

Another embodiment comprises about 0.05% cyclosporin A, Cottonseed oil, and an alcohol ethoxylate.

Another embodiment comprises about 0.05% cyclosporin A, Safflower oil, and an alcohol ethoxylate.

Another embodiment comprises about 0.05% cyclosporin A, Maize oil, and an alcohol ethoxylate.

Another embodiment comprises about 0.05% cyclosporin A, Linseed oil, and an alcohol ethoxylate.

Another embodiment comprises about 0.05% cyclosporin A, Rapeseed oil, and an alcohol ethoxylate.

Another embodiment comprises about 0.05% cyclosporin A, Soybean oil, and an alcohol ethoxylate.

Another embodiment comprises about 0.05% cyclosporin A, Olive oil, and an alcohol ethoxylate.

Another embodiment comprises about 0.05% cyclosporin A, Caraway oil, and an alcohol ethoxylate.

Another embodiment comprises about 0.05% cyclosporin A, Rosemary oil, and an alcohol ethoxylate.

Another embodiment comprises about 0.05% cyclosporin A, Peanut oil, and an alcohol ethoxylate.

Another embodiment comprises about 0.05% cyclosporin A, Peppermint oil, and an alcohol ethoxylate.

Another embodiment comprises about 0.05% cyclosporin A, Sunflower oil, and an alcohol ethoxylate.

Another embodiment comprises about 0.05% cyclosporin A, Eucalyptus oil, and an alcohol ethoxylate.

Another embodiment comprises about 0.05% cyclosporin A, Sesame oil, and an alcohol ethoxylate.

Another embodiment comprises about 0.05% cyclosporin A, an oil having a specific gravity below 0.9, and an alcohol ethoxylate.

Another embodiment comprises about 0.05% cyclosporin A, Mineral oil, and an alcohol ethoxylate.

Another embodiment comprises about 0.05% cyclosporin A, Coriander oil, and an alcohol ethoxylate.

Another embodiment comprises about 0.05% cyclosporin A, Lavender oil, and an alcohol ethoxylate.

Another embodiment comprises about 0.05% cyclosporin A, Citronella oil, and an alcohol ethoxylate.

Another embodiment comprises about 0.05% cyclosporin A, Juniper oil, and an alcohol ethoxylate.

Another embodiment comprises about 0.05% cyclosporin A, Lemon oil, and an alcohol ethoxylate.

Another embodiment comprises about 0.05% cyclosporin A, Orange oil, and an alcohol ethoxylate.

Another embodiment comprises about 0.05% cyclosporin A, Clary sage oil, and an alcohol ethoxylate.

Another embodiment comprises about 0.05% cyclosporin A, Nutmeg oil, and an alcohol ethoxylate.

Another embodiment comprises about 0.05% cyclosporin A, Tea tree oil, and an alcohol ethoxylate.

Another embodiment comprises about 0.05% cyclosporin A, an oil having a specific gravity from 0.95 to 1.07, and an alkylphenol ethoxylate.

Another embodiment comprises about 0.05% cyclosporin A, Anise oil, and an alkylphenol ethoxylate.

Another embodiment comprises about 0.05% cyclosporin A, Castor oil, and an alkylphenol ethoxylate.

Another embodiment comprises about 0.05% cyclosporin A, Clove oil, and an alkylphenol ethoxylate.

Another embodiment comprises about 0.05% cyclosporin A, Cassia oil, and an alkylphenol ethoxylate.

Another embodiment comprises about 0.05% cyclosporin A, Cinnamon oil, and an alkylphenol ethoxylate.

Another embodiment comprises about 0.05% cyclosporin A, an oil having a specific gravity from 0.90 to 0.95, and an alkylphenol ethoxylate.

Another embodiment comprises about 0.05% cyclosporin A, Almond oil, and an alkylphenol ethoxylate.

Another embodiment comprises about 0.05% cyclosporin A, Corn oil, and an alkylphenol ethoxylate.

Another embodiment comprises about 0.05% cyclosporin A, Arachis oil, and an alkylphenol ethoxylate.

Another embodiment comprises about 0.05% cyclosporin A, Cottonseed oil, and an alkylphenol ethoxylate.

Another embodiment comprises about 0.05% cyclosporin A, Safflower oil, and an alkylphenol ethoxylate.

Another embodiment comprises about 0.05% cyclosporin A, Maize oil, and an alkylphenol ethoxylate.

Another embodiment comprises about 0.05% cyclosporin A, Linseed oil, and an alkylphenol ethoxylate.

Another embodiment comprises about 0.05% cyclosporin A, Rapeseed oil, and an alkylphenol ethoxylate.

Another embodiment comprises about 0.05% cyclosporin A, Soybean oil, and an alkylphenol ethoxylate.

Another embodiment comprises about 0.05% cyclosporin A, Olive oil, and an alkylphenol ethoxylate.

Another embodiment comprises about 0.05% cyclosporin A, Caraway oil, and an alkylphenol ethoxylate.

Another embodiment comprises about 0.05% cyclosporin A, Rosemary oil, and an alkylphenol ethoxylate.

Another embodiment comprises about 0.05% cyclosporin A, Peanut oil, and an alkylphenol ethoxylate.

Another embodiment comprises about 0.05% cyclosporin A, Peppermint oil, and an alkylphenol ethoxylate.

Another embodiment comprises about 0.05% cyclosporin A, Sunflower oil, and an alkylphenol ethoxylate.

Another embodiment comprises about 0.05% cyclosporin A, Eucalyptus oil, and an alkylphenol ethoxylate.

Another embodiment comprises about 0.05% cyclosporin A, Sesame oil, and an alkylphenol ethoxylate.

Another embodiment comprises about 0.05% cyclosporin A, an oil having a specific gravity below 0.9, and an alkylphenol ethoxylate.

Another embodiment comprises about 0.05% cyclosporin A, Mineral oil, and an alkylphenol ethoxylate.

Another embodiment comprises about 0.05% cyclosporin A, Coriander oil, and an alkylphenol ethoxylate.

Another embodiment comprises about 0.05% cyclosporin A, Lavender oil, and an alkylphenol ethoxylate.

Another embodiment comprises about 0.05% cyclosporin A, Citronella oil, and an alkylphenol ethoxylate.

Another embodiment comprises about 0.05% cyclosporin A, Juniper oil, and an alkylphenol ethoxylate.

Another embodiment comprises about 0.05% cyclosporin A, Lemon oil, and an alkylphenol ethoxylate.

Another embodiment comprises about 0.05% cyclosporin A, Orange oil, and an alkylphenol ethoxylate.

Another embodiment comprises about 0.05% cyclosporin A, Clary sage oil, and an alkylphenol ethoxylate.

Another embodiment comprises about 0.05% cyclosporin A, Nutmeg oil, and an alkylphenol ethoxylate.

Another embodiment comprises about 0.05% cyclosporin A, Tea tree oil, and an alkylphenol ethoxylate.

Another embodiment comprises about 0.05% cyclosporin A, an oil having a specific gravity from 0.95 to 1.07, and an alkyl glycoside.

Another embodiment comprises about 0.05% cyclosporin A, Anise oil, and an alkyl glycoside.

Another embodiment comprises about 0.05% cyclosporin A, Castor oil, and an alkyl glycoside.

Another embodiment comprises about 0.05% cyclosporin A, Clove oil, and an alkyl glycoside.

Another embodiment comprises about 0.05% cyclosporin A, Cassia oil, and an alkyl glycoside.

Another embodiment comprises about 0.05% cyclosporin A, Cinnamon oil, and an alkyl glycoside..

Another embodiment comprises about 0.05% cyclosporin A, an oil having a specific gravity from 0.90 to 0.95, and an alkyl glycoside.

Another embodiment comprises about 0.05% cyclosporin A, Almond oil, and an alkyl glycoside.

Another embodiment comprises about 0.05% cyclosporin A, Corn oil, and an alkyl glycoside.

Another embodiment comprises about 0.05% cyclosporin A, Arachis oil, and an alkyl glycoside.

Another embodiment comprises about 0.05% cyclosporin A, Cottonseed oil, and an alkyl glycoside.

Another embodiment comprises about 0.05% cyclosporin A, Safflower oil, and an alkyl glycoside.

Another embodiment comprises about 0.05% cyclosporin A, Maize oil, and an alkyl glycoside.

Another embodiment comprises about 0.05% cyclosporin A, Linseed oil, and an alkyl glycoside.

Another embodiment comprises about 0.05% cyclosporin A, Rapeseed oil, and an alkyl glycoside.

Another embodiment comprises about 0.05% cyclosporin A, Soybean oil, and an alkyl glycoside.

Another embodiment comprises about 0.05% cyclosporin A, Olive oil, and an alkyl glycoside.

Another embodiment comprises about 0.05% cyclosporin A, Caraway oil, and an alkyl glycoside.

Another embodiment comprises about 0.05% cyclosporin A, Rosemary oil, and an alkyl glycoside.

Another embodiment comprises about 0.05% cyclosporin A, Peanut oil, and an alkyl glycoside.

Another embodiment comprises about 0.05% cyclosporin A, Peppermint oil, and an alkyl glycoside.

Another embodiment comprises about 0.05% cyclosporin A, Sunflower oil, and an alkyl glycoside.

Another embodiment comprises about 0.05% cyclosporin A, Eucalyptus oil, and an alkyl glycoside.

Another embodiment comprises about 0.05% cyclosporin A, Sesame oil, and an alkyl glycoside.

Another embodiment comprises about 0.05% cyclosporin A, an oil having a specific gravity below 0.9, and an alkyl glycoside.

Another embodiment comprises about 0.05% cyclosporin A, Mineral oil, and an alkyl glycoside.

Another embodiment comprises about 0.05% cyclosporin A, Coriander oil, and an alkyl glycoside.

Another embodiment comprises about 0.05% cyclosporin A, Lavender oil, and an alkyl glycoside.

Another embodiment comprises about 0.05% cyclosporin A, Citronella oil, and an alkyl glycoside.

Another embodiment comprises about 0.05% cyclosporin A, Juniper oil, and an alkyl glycoside.

Another embodiment comprises about 0.05% cyclosporin A, Lemon oil, and an alkyl glycoside.

Another embodiment comprises about 0.05% cyclosporin A, Orange oil, and an alkyl glycoside.

Another embodiment comprises about 0.05% cyclosporin A, Clary sage oil, and an alkyl glycoside.

Another embodiment comprises about 0.05% cyclosporin A, Nutmeg oil, and an alkyl glycoside.

Another embodiment comprises about 0.05% cyclosporin A, Tea tree oil, and an alkyl glycoside.

Another embodiment comprises about 0.05% cyclosporin A, an oil having a specific gravity from 0.95 to 1.07, and an alkyl polyglycoside.

Another embodiment comprises about 0.05% cyclosporin A, Anise oil, and an alkyl polyglycoside.

Another embodiment comprises about 0.05% cyclosporin A, Castor oil, and an alkyl polyglycoside.

Another embodiment comprises about 0.05% cyclosporin A, Clove oil, and an alkyl polyglycoside.

Another embodiment comprises about 0.05% cyclosporin A, Cassia oil, and an alkyl polyglycoside.

Another embodiment comprises about 0.05% cyclosporin A, Cinnamon oil, and an alkyl polyglycoside.

Another embodiment comprises about 0.05% cyclosporin A, an oil having a specific gravity from 0.90 to 0.95, and an alkyl polyglycoside.

Another embodiment comprises about 0.05% cyclosporin A, Almond oil, and an alkyl polyglycoside.

Another embodiment comprises about 0.05% cyclosporin A, Corn oil, and an alkyl polyglycoside.

Another embodiment comprises about 0.05% cyclosporin A, Arachis oil, and an alkyl polyglycoside.

Another embodiment comprises about 0.05% cyclosporin A, Cottonseed oil, and an alkyl polyglycoside.

Another embodiment comprises about 0.05% cyclosporin A, Safflower oil, and an alkyl polyglycoside.

Another embodiment comprises about 0.05% cyclosporin A, Maize oil, and an alkyl polyglycoside.

Another embodiment comprises about 0.05% cyclosporin A, Linseed oil, and an alkyl polyglycoside.

Another embodiment comprises about 0.05% cyclosporin A, Rapeseed oil, and an alkyl polyglycoside.

Another embodiment comprises about 0.05% cyclosporin A, Soybean oil, and an alkyl polyglycoside.

Another embodiment comprises about 0.05% cyclosporin A, Olive oil, and an alkyl polyglycoside.

Another embodiment comprises about 0.05% cyclosporin A, Caraway oil, and an alkyl polyglycoside.

Another embodiment comprises about 0.05% cyclosporin A, Rosemary oil, and an alkyl polyglycoside.

Another embodiment comprises about 0.05% cyclosporin A, Peanut oil, and an alkyl polyglycoside.

Another embodiment comprises about 0.05% cyclosporin A, Peppermint oil, and an alkyl polyglycoside.

Another embodiment comprises about 0.05% cyclosporin A, Sunflower oil, and an alkyl polyglycoside.

Another embodiment comprises about 0.05% cyclosporin A, Eucalyptus oil, and an alkyl polyglycoside.

Another embodiment comprises about 0.05% cyclosporin A, Sesame oil, and an alkyl polyglycoside.

Another embodiment comprises about 0.05% cyclosporin A, an oil having a specific gravity below 0.9, and an alkyl polyglycoside.

Another embodiment comprises about 0.05% cyclosporin A, Mineral oil, and an alkyl polyglycoside.

Another embodiment comprises about 0.05% cyclosporin A, Coriander oil, and an alkyl polyglycoside.

Another embodiment comprises about 0.05% cyclosporin A, Lavender oil, and an alkyl polyglycoside.

Another embodiment comprises about 0.05% cyclosporin A, Citronella oil, and an alkyl polyglycoside.

Another embodiment comprises about 0.05% cyclosporin A, Juniper oil, and an alkyl polyglycoside.

Another embodiment comprises about 0.05% cyclosporin A, Lemon oil, and an alkyl polyglycoside.

Another embodiment comprises about 0.05% cyclosporin A, Orange oil, and an alkyl polyglycoside.

Another embodiment comprises about 0.05% cyclosporin A, Clary sage oil, and an alkyl polyglycoside.

Another embodiment comprises about 0.05% cyclosporin A, Nutmeg oil, and an alkyl polyglycoside.

Another embodiment comprises about 0.05% cyclosporin A, Tea tree oil, and an alkyl polyglycoside.

Another embodiment comprises about 0.05% cyclosporin A, an oil having a specific gravity from 0.95 to 1.07, and a fatty alcohol.

Another embodiment comprises about 0.05% cyclosporin A, Anise oil, and a fatty alcohol.

Another embodiment comprises about 0.05% cyclosporin A, Castor oil, and a fatty alcohol.

Another embodiment comprises about 0.05% cyclosporin A, Clove oil, and a fatty alcohol.

Another embodiment comprises about 0.05% cyclosporin A, Cassia oil, and a fatty alcohol.

Another embodiment comprises about 0.05% cyclosporin A, Cinnamon oil, and a fatty alcohol.

Another embodiment comprises about 0.05% cyclosporin A, an oil having a specific gravity from 0.90 to 0.95, and a fatty alcohol.

Another embodiment comprises about 0.05% cyclosporin A, Almond oil, and a fatty alcohol.

Another embodiment comprises about 0.05% cyclosporin A, Corn oil, and a fatty alcohol.

Another embodiment comprises about 0.05% cyclosporin A, Arachis oil, and a fatty alcohol.

Another embodiment comprises about 0.05% cyclosporin A, Cottonseed oil, and a fatty alcohol.

Another embodiment comprises about 0.05% cyclosporin A, Safflower oil, and a fatty alcohol.

Another embodiment comprises about 0.05% cyclosporin A, Maize oil, and a fatty alcohol.

Another embodiment comprises about 0.05% cyclosporin A, Linseed oil, and a fatty alcohol.

Another embodiment comprises about 0.05% cyclosporin A, Rapeseed oil, and a fatty alcohol.

Another embodiment comprises about 0.05% cyclosporin A, Soybean oil, and a fatty alcohol.

Another embodiment comprises about 0.05% cyclosporin A, Olive oil, and a fatty alcohol.

Another embodiment comprises about 0.05% cyclosporin A, Caraway oil, and a fatty alcohol.

Another embodiment comprises about 0.05% cyclosporin A, Rosemary oil, and a fatty alcohol.

Another embodiment comprises about 0.05% cyclosporin A, Peanut oil, and a fatty alcohol.

Another embodiment comprises about 0.05% cyclosporin A, Peppermint oil, and a fatty alcohol.

Another embodiment comprises about 0.05% cyclosporin A, Sunflower oil, and a fatty alcohol.

Another embodiment comprises about 0.05% cyclosporin A, Eucalyptus oil, and a fatty alcohol.

Another embodiment comprises about 0.05% cyclosporin A, Sesame oil, and a fatty alcohol.

Another embodiment comprises about 0.05% cyclosporin A, an oil having a specific gravity below 0.9, and a fatty alcohol.

Another embodiment comprises about 0.05% cyclosporin A, Mineral oil, and a fatty alcohol.

Another embodiment comprises about 0.05% cyclosporin A, Coriander oil, and a fatty alcohol.

Another embodiment comprises about 0.05% cyclosporin A, Lavender oil, and a fatty alcohol.

Another embodiment comprises about 0.05% cyclosporin A, Citronella oil, and a fatty alcohol.

Another embodiment comprises about 0.05% cyclosporin A, Juniper oil, and a fatty alcohol.

Another embodiment comprises about 0.05% cyclosporin A, Lemon oil, and a fatty alcohol.

Another embodiment comprises about 0.05% cyclosporin A, Orange oil, and a fatty alcohol.

Another embodiment comprises about 0.05% cyclosporin A, Clary sage oil, and a fatty alcohol.

Another embodiment comprises about 0.05% cyclosporin A, Nutmeg oil, and a fatty alcohol.

Another embodiment comprises about 0.05% cyclosporin A, Tea tree oil, and a fatty alcohol.

Another embodiment comprises about 0.05% cyclosporin A, an oil having a specific gravity from 0.95 to 1.07, and a cellulose derivative.

Another embodiment comprises about 0.05% cyclosporin A, Anise oil, and a cellulose derivative.

Another embodiment comprises about 0.05% cyclosporin A, Castor oil, and a cellulose derivative.

Another embodiment comprises about 0.05% cyclosporin A, Clove oil, and a cellulose derivative.

Another embodiment comprises about 0.05% cyclosporin A, Cassia oil, and a cellulose derivative.

Another embodiment comprises about 0.05% cyclosporin A, Cinnamon oil, and a cellulose derivative.

Another embodiment comprises about 0.05% cyclosporin A, an oil having a specific gravity from 0.90 to 0.95, and a cellulose derivative.

Another embodiment comprises about 0.05% cyclosporin A, Almond oil, and a cellulose derivative.

Another embodiment comprises about 0.05% cyclosporin A, Corn oil, and a cellulose derivative.

Another embodiment comprises about 0.05% cyclosporin A, Arachis oil, and a cellulose derivative.

Another embodiment comprises about 0.05% cyclosporin A, Cottonseed oil, and a cellulose derivative.

Another embodiment comprises about 0.05% cyclosporin A, Safflower oil, and a cellulose derivative.

Another embodiment comprises about 0.05% cyclosporin A, Maize oil, and a cellulose derivative.

Another embodiment comprises about 0.05% cyclosporin A, Linseed oil, and a cellulose derivative.

Another embodiment comprises about 0.05% cyclosporin A, Rapeseed oil, and a cellulose derivative.

Another embodiment comprises about 0.05% cyclosporin A, Soybean oil, and a cellulose derivative.

Another embodiment comprises about 0.05% cyclosporin A, Olive oil, and a cellulose derivative.

Another embodiment comprises about 0.05% cyclosporin A, Caraway oil, and a cellulose derivative.

Another embodiment comprises about 0.05% cyclosporin A, Rosemary oil, and a cellulose derivative.

Another embodiment comprises about 0.05% cyclosporin A, Peanut oil, and a cellulose derivative.

Another embodiment comprises about 0.05% cyclosporin A, Peppermint oil, and a cellulose derivative.

Another embodiment comprises about 0.05% cyclosporin A, Sunflower oil, and a cellulose derivative.

Another embodiment comprises about 0.05% cyclosporin A, Eucalyptus oil, and a cellulose derivative.

Another embodiment comprises about 0.05% cyclosporin A, Sesame oil, and a cellulose derivative.

Another embodiment comprises about 0.05% cyclosporin A, an oil having a specific gravity below 0.9, and a cellulose derivative.

Another embodiment comprises about 0.05% cyclosporin A, Mineral oil, and a cellulose derivative.

Another embodiment comprises about 0.05% cyclosporin A, Coriander oil, and a cellulose derivative.

Another embodiment comprises about 0.05% cyclosporin A, Lavender oil, and a cellulose derivative.

Another embodiment comprises about 0.05% cyclosporin A, Citronella oil, and a cellulose derivative.

Another embodiment comprises about 0.05% cyclosporin A, Juniper oil, and a cellulose derivative.

Another embodiment comprises about 0.05% cyclosporin A, Lemon oil, and a cellulose derivative.

Another embodiment comprises about 0.05% cyclosporin A, Orange oil, and a cellulose derivative.

Another embodiment comprises about 0.05% cyclosporin A, Clary sage oil, and a cellulose derivative.

Another embodiment comprises about 0.05% cyclosporin A, Nutmeg oil, and a cellulose derivative.

Another embodiment comprises about 0.05% cyclosporin A, Tea tree oil, and a cellulose derivative.

Another embodiment comprises about 0.05% cyclosporin A, an oil having a specific gravity from 0.95 to 1.07, and hydroxypropylmethyl cellulose.

Another embodiment comprises about 0.05% cyclosporin A, Anise oil, and hydroxypropylmethyl cellulose.

Another embodiment comprises about 0.05% cyclosporin A, Castor oil, and hydroxypropylmethyl cellulose.

Another embodiment comprises about 0.05% cyclosporin A, Clove oil, and hydroxypropylmethyl cellulose.

Another embodiment comprises about 0.05% cyclosporin A, Cassia oil, and hydroxypropylmethyl cellulose.

Another embodiment comprises about 0.05% cyclosporin A, Cinnamon oil, and hydroxypropylmethyl cellulose.

Another embodiment comprises about 0.05% cyclosporin A, an oil having a specific gravity from 0.90 to 0.95, and hydroxypropylmethyl cellulose.

Another embodiment comprises about 0.05% cyclosporin A, Almond oil, and hydroxypropylmethyl cellulose.

Another embodiment comprises about 0.05% cyclosporin A, Corn oil, and hydroxypropylmethyl cellulose.

Another embodiment comprises about 0.05% cyclosporin A, Arachis oil, and hydroxypropylmethyl cellulose.

Another embodiment comprises about 0.05% cyclosporin A, Cottonseed oil, and hydroxypropylmethyl cellulose.

Another embodiment comprises about 0.05% cyclosporin A, Safllower oil, and hydroxypropylmethyl cellulose.

Another embodiment comprises about 0.05% cyclosporin A, Maize oil, and hydroxypropylmethyl cellulose.

Another embodiment comprises about 0.05% cyclosporin A, Linseed oil, and hydroxypropylmethyl cellulose.

Another embodiment comprises about 0.05% cyclosporin A, Rapeseed oil, and hydroxypropylmethyl cellulose.

Another embodiment comprises about 0.05% cyclosporin A, Soybean oil, and hydroxypropylmethyl cellulose.

Another embodiment comprises about 0.05% cyclosporin A, Olive oil, and hydroxypropylmethyl cellulose.

Another embodiment comprises about 0.05% cyclospdrin A, Caraway oil, and hydroxypropylmethyl cellulose.

Another embodiment comprises about 0.05% cyclosporin A, Rosemary oil, and hydroxypropylmethyl cellulose.

Another embodiment comprises about 0.05% cyclosporin A, Peanut oil, and hydroxypropylmethyl cellulose.

Another embodiment comprises about 0.05% cyclosporin A, Peppermint oil, and hydroxypropylmethyl cellulose.

Another embodiment comprises about 0.05% cyclosporin A, Sunflower oil, and hydroxypropylmethyl cellulose.

Another embodiment comprises about 0.05% cyclosporin A, Eucalyptus oil, and hydroxypropylmethyl cellulose.

Another embodiment comprises about 0.05% cyclosporin A, Sesame oil, and hydroxypropylmethyl cellulose.

Another embodiment comprises about 0.05% cyclosporin A, an oil having a specific gravity below 0.9, and hydroxypropylmethyl cellulose.

Another embodiment comprises about 0.05% cyclosporin A, Mineral oil, and hydroxypropylmethyl cellulose.

Another embodiment comprises about 0.05% cyclosporin A, Coriander oil, and hydroxypropylmethyl cellulose.

Another embodiment comprises about 0.05% cyclosporin A, Lavender oil, and hydroxypropylmethyl cellulose.

Another embodiment comprises about 0.05% cyclosporin A, Citronella oil, and hydroxypropylmethyl cellulose.

Another embodiment comprises about 0.05% cyclosporin A, Juniper oil, and hydroxypropylmethyl cellulose.

Another embodiment comprises about 0.05% cyclosporin A, Lemon oil, and hydroxypropylmethyl cellulose.

Another embodiment comprises about 0.05% cyclosporin A, Orange oil, and hydroxypropylmethyl cellulose.

Another embodiment comprises about 0.05% cyclosporin A, Clary sage oil, and hydroxypropylmethyl cellulose.

Another embodiment comprises about 0.05% cyclosporin A, Nutmeg oil, and hydroxypropylmethyl cellulose.

Another embodiment comprises about 0.05% cyclosporin A, Tea tree oil, and hydroxypropylmethyl cellulose.

Another embodiment comprises about 0.05% cyclosporin A, an oil having a specific gravity from 0.95 to 1.07, and carboxymethyl cellulose.

Another embodiment comprises about 0.05% cyclosporin A, Anise oil, and carboxymethyl cellulose.

Another embodiment comprises about 0.05% cyclosporin A, Castor oil, and carboxymethyl cellulose.

Another embodiment comprises about 0.05% cyclosporin A, Clove oil, and carboxymethyl cellulose.

Another embodiment comprises about 0.05% cyclosporin A, Cassia oil, and carboxymethyl cellulose.

Another embodiment comprises about 0.05% cyclosporin A, Cinnamon oil, and carboxymethyl cellulose.

Another embodiment comprises about 0.05% cyclosporin A, an oil having a specific gravity from 0.90 to 0.95, and carboxymethyl cellulose.

Another embodiment comprises about 0.05% cyclosporin A, Almond oil, and carboxymethyl cellulose.

Another embodiment comprises about 0.05% cyclosporin A, Corn oil, and carboxymethyl cellulose.

Another embodiment comprises about 0.05% cyclosporin A, Arachis oil, and carboxymethyl cellulose.

Another embodiment comprises about 0.05% cyclosporin A, Cottonseed oil, and carboxymethyl cellulose.

Another embodiment comprises about 0.05% cyclosporin A, Safllower oil, and carboxymethyl cellulose.

Another embodiment comprises about 0.05% cyclosporin A, Maize oil, and carboxymethyl cellulose.

Another embodiment comprises about 0.05% cyclosporin A, Linseed oil, and carboxymethyl cellulose.

Another embodiment comprises about 0.05% cyclosporin A, Rapeseed oil, and carboxymethyl cellulose.

Another embodiment comprises about 0.05% cyclosporin A, Soybean oil, and carboxymethyl cellulose.

Another embodiment comprises about 0.05% cyclosporin A, Olive oil, and carboxymethyl cellulose.

Another embodiment comprises about 0.05% cyclosporin A, Caraway oil, and carboxymethyl cellulose.

Another embodiment comprises about 0.05% cyclosporin A, Rosemary oil, and carboxymethyl cellulose.

Another embodiment comprises about 0.05% cyclosporin A, Peanut oil, and carboxymethyl cellulose.

Another embodiment comprises about 0.05% cyclosporin A, Peppermint oil, and carboxymethyl cellulose.

Another embodiment comprises about 0.05% cyclosporin A, Sunflower oil, and carboxymethyl cellulose.

Another embodiment comprises about 0.05% cyclosporin A, Eucalyptus oil, and carboxymethyl cellulose.

Another embodiment comprises about 0.05% cyclosporin A, Sesame oil, and carboxymethyl cellulose.

Another embodiment comprises about 0.05% cyclosporin A, an oil having a specific gravity below 0.9, and carboxymethyl cellulose.

Another embodiment comprises about 0.05% cyclosporin A, Mineral oil, and carboxymethyl cellulose.

Another embodiment comprises about 0.05% cyclosporin A, Coriander oil, and carboxymethyl cellulose.

Another embodiment comprises about 0.05% cyclosporin A, Lavender oil, and carboxymethyl cellulose.

Another embodiment comprises about 0.05% cyclosporin A, Citronella oil, and carboxymethyl cellulose.

Another embodiment comprises about 0.05% cyclosporin A, Juniper oil, and carboxymethyl cellulose.

Another embodiment comprises about 0.05% cyclosporin A, Lemon oil, and carboxymethyl cellulose.

Another embodiment comprises about 0.05% cyclosporin A, Orange oil, and carboxymethyl cellulose.

Another embodiment comprises about 0.05% cyclosporin A, Clary sage oil, and carboxymethyl cellulose.

Another embodiment comprises about 0.05% cyclosporin A, Nutmeg oil, and carboxymethyl cellulose.

Another embodiment comprises about 0.05% cyclosporin A, Tea tree oil, and carboxymethyl cellulose.

Another embodiment comprises about 0.05% cyclosporin A, an oil having a specific gravity from 0.95 to 1.07, and a polyacrylic acid.

Another embodiment comprises about 0.05% cyclosporin A, Anise oil, and a polyacrylic acid.

Another embodiment comprises about 0.05% cyclosporin A, Castor oil, and a polyacrylic acid.

Another embodiment comprises about 0.05% cyclosporin A, Clove oil, and a polyacrylic acid.

Another embodiment comprises about 0.05% cyclosporin A, Cassia oil, and a polyacrylic acid.

Another embodiment comprises about 0.05% cyclosporin A, Cinnamon oil, and a polyacrylic acid.

Another embodiment comprises about 0.05% cyclosporin A, an oil having a specific gravity from 0.90 to 0.95, and a polyacrylic acid.

Another embodiment comprises about 0.05% cyclosporin A, Almond oil, and a polyacrylic acid.

Another embodiment comprises about 0.05% cyclosporin A, Corn oil, and a polyacrylic acid.

Another embodiment comprises about 0.05% cyclosporin A, Arachis oil, and a polyacrylic acid.

Another embodiment comprises about 0.05% cyclosporin A, Cottonseed oil, and a polyacrylic acid.

Another embodiment comprises about 0.05% cyclosporin A, Safflower oil, and a polyacrylic acid.

Another embodiment comprises about 0.05% cyclosporin A, Maize oil, and a polyacrylic acid.

Another embodiment comprises about 0.05% cyclosporin A, Linseed oil, and a polyacrylic acid.

Another embodiment comprises about 0.05% cyclosporin A, Rapeseed oil, and a polyacrylic acid.

Another embodiment comprises about 0.05% cyclosporin A, Soybean oil, and a polyacrylic acid.

Another embodiment comprises about 0.05% cyclosporin A, Olive oil, and a polyacrylic acid.

Another embodiment comprises about 0.05% cyclosporin A, Caraway oil, and a polyacrylic acid.

Another embodiment comprises about 0.05% cyclosporin A, Rosemary oil, and a polyacrylic acid.

Another embodiment comprises about 0.05% cyclosporin A, Peanut oil, and a polyacrylic acid.

Another embodiment comprises about 0.05% cyclosporin A, Peppermint oil, and a polyacrylic acid.

Another embodiment comprises about 0.05% cyclosporin A, Sunflower oil, and a polyacrylic acid.

Another embodiment comprises about 0.05% cyclosporin A, Eucalyptus oil, and a polyacrylic acid.

Another embodiment comprises about 0.05% cyclosporin A, Sesame oil, and a polyacrylic acid.

Another embodiment comprises about 0.05% cyclosporin A, an oil having a specific gravity below 0.9, and a polyacrylic acid.

Another embodiment comprises about 0.05% cyclosporin A, Mineral oil, and a polyacrylic acid.

Another embodiment comprises about 0.05% cyclosporin A, Coriander oil, and a polyacrylic acid.

Another embodiment comprises about 0.05% cyclosporin A, Lavender oil, and a polyacrylic acid.

Another embodiment comprises about 0.05% cyclosporin A, Citronella oil, and a polyacrylic acid.

Another embodiment comprises about 0.05% cyclosporin A, Juniper oil, and a polyacrylic acid.

Another embodiment comprises about 0.05% cyclosporin A, Lemon oil, and a polyacrylic acid.

Another embodiment comprises about 0.05% cyclosporin A, Orange oil, and a polyacrylic acid.

Another embodiment comprises about 0.05% cyclosporin A, Clary sage oil, and a polyacrylic acid.

Another embodiment comprises about 0.05% cyclosporin A, Nutmeg oil, and a polyacrylic acid.-

Another embodiment comprises about 0.05% cyclosporin A, Tea tree oil, and a polyacrylic acid.

Another embodiment comprises about 0.05% cyclosporin A, an oil having a specific gravity from 0.95 to 1.07, and a Carbomer.

Another embodiment comprises about 0.05% cyclosporin A, Anise oil, and a Carbomer.

Another embodiment comprises about 0.05% cyclosporin A, Castor oil, and a Carbomer.

Another embodiment comprises about 0.05% cyclosporin A, Clove oil, and a Carbomer.

Another embodiment comprises about 0.05% cyclosporin A, Cassia oil, and a Carbomer.

Another embodiment comprises about 0.05% cyclosporin A, Cinnamon oil, and a Carbomer.

Another embodiment comprises about 0.05% cyclosporin A, an oil having a specific gravity from 0.90 to 0.95, and a Carbomer.

Another embodiment comprises about 0.05% cyclosporin A, Almond oil, and a Carbomer.

Another embodiment comprises about 0.05% cyclosporin A, Corn oil, and a Carbomer.

Another embodiment comprises about 0.05% cyclosporin A, Arachis oil, and a Carbomer.

Another embodiment comprises about 0.05% cyclosporin A, Cottonseed oil, and a Carbomer.

Another embodiment comprises about 0.05% cyclosporin A, Safflower oil, and a Carbomer.

Another embodiment comprises about 0.05% cyclosporin A, Maize oil, and a Carbomer.

Another embodiment comprises about 0.05% cyclosporin A, Linseed oil, and a Carbomer.

Another embodiment comprises about 0.05% cyclosporin A, Rapeseed oil, and a Carbomer.

Another embodiment comprises about 0.05% cyclosporin A, Soybean oil, and a Carbomer.

Another embodiment comprises about 0.05% cyclosporin A, Olive oil, and a Carbomer.

Another embodiment comprises about 0.05% cyclosporin A, Caraway oil, and a Carbomer.

Another embodiment comprises about 0.05% cyclosporin A, Rosemary oil, and a Carbomer.

Another embodiment comprises about 0.05% cyclosporin A, Peanut oil, and a Carbomer.

Another embodiment comprises about 0.05% cyclosporin A, Peppermint oil, and a Carbomer.

Another embodiment comprises about 0.05% cyclosporin A, Sunflower oil, and a Carbomer.

Another embodiment comprises about 0.05% cyclosporin A, Eucalyptus oil, and a Carbomer.

Another embodiment comprises about 0.05% cyclosporin A, Sesame oil, and a Carbomer.

Another embodiment comprises about 0.05% cyclosporin A, an oil having a specific gravity below 0.9, and a Carbomer.

Another embodiment comprises about 0.05% cyclosporin A, Mineral oil, and a Carbomer.

Another embodiment comprises about 0.05% cyclosporin A, Coriander oil, and a Carbomer.

Another embodiment comprises about 0.05% cyclosporin A, Lavender oil, and a Carbomer.

Another embodiment comprises about 0.05% cyclosporin A, Citronella oil, and a Carbomer.

Another embodiment comprises about 0.05% cyclosporin A, Juniper oil, and a Carbomer.

Another embodiment comprises about 0.05% cyclosporin A, Lemon oil, and a Carbomer.

Another embodiment comprises about 0.05% cyclosporin A, Orange oil, and a Carbomer.

Another embodiment comprises about 0.05% cyclosporin A, Clary sage oil, and a Carbomer.

Another embodiment comprises about 0.05% cyclosporin A, Nutmeg oil, and a Carbomer.

Another embodiment comprises about 0.05% cyclosporin A, Tea tree oil, and a Carbomer.

Another embodiment comprises about 0.05% cyclosporin A, an oil having a specific gravity from 0.95 to 1.07, and a phosphalipid.

Another embodiment comprises about 0.05% cyclosporin A, Anise oil, and a phosphalipid.

Another embodiment comprises about 0.05% cyclosporin A, Castor oil, and a phosphalipid.

Another embodiment comprises about 0.05% cyclosporin A, Clove oil, and a phosphalipid.

Another embodiment comprises about 0.05% cyclosporin A, Cassia oil, and a phosphalipid.

Another embodiment comprises about 0.05% cyclosporin A, Cinnamon oil, and a phosphalipid.

Another embodiment comprises about 0.05% cyclosporin A, an oil having a specific gravity from 0.90 to 0.95, and a phosphalipid.

Another embodiment comprises about 0.05% cyclosporin A, Almond oil, and a phosphalipid.

Another embodiment comprises about 0.05% cyclosporin A, Corn oil, and a phosphalipid.

Another embodiment comprises about 0.05% cyclosporin A, Arachis oil, and a phosphalipid.

Another embodiment comprises about 0.05% cyclosporin A, Cottonseed oil, and a phosphalipid.

Another embodiment comprises about 0.05% cyclosporin A, Safflower oil, and a phosphalipid.

Another embodiment comprises about 0.05% cyclosporin A, Maize oil, and a phosphalipid.

Another embodiment comprises about 0.05% cyclosporin A, Linseed oil, and a phosphalipid.

Another embodiment comprises about 0.05% cyclosporin A, Rapeseed oil, and a phosphalipid.

Another embodiment comprises about 0.05% cyclosporin A, Soybean oil, and a phosphalipid.

Another embodiment comprises about 0.05% cyclosporin A, Olive oil, and a phosphalipid.

Another embodiment comprises about 0.05% cyclosporin A, Caraway oil, and a phosphalipid.

Another embodiment comprises about 0.05% cyclosporin A, Rosemary oil, and a phosphalipid.

Another embodiment comprises about 0.05% cyclosporin A, Peanut oil, and a phosphalipid.

Another embodiment comprises about 0.05% cyclosporin A, Peppermint oil, and a phosphalipid.

Another embodiment comprises about 0.05% cyclosporin A, Sunflower oil, and a phosphalipid.

Another embodiment comprises about 0.05% cyclosporin A, Eucalyptus oil, and a phosphalipid.

Another embodiment comprises about 0.05% cyclosporin A, Sesame oil, and a phosphalipid.

Another embodiment comprises about 0.05% cyclosporin A, an oil having a specific gravity below 0.9, and a phosphalipid.

Another embodiment comprises about 0.05% cyclosporin A, Mineral oil, and a phosphalipid.

Another embodiment comprises about 0.05% cyclosporin A, Coriander oil, and a phosphalipid.

Another embodiment comprises about 0.05% cyclosporin A, Lavender oil, and a phosphalipid.

Another embodiment comprises about 0.05% cyclosporin A, Citronella oil, and a phosphalipid.

Another embodiment comprises about 0.05% cyclosporin A, Juniper oil, and a phosphalipid.

Another embodiment comprises about 0.05% cyclosporin A, Lemon oil, and a phosphalipid.

Another embodiment comprises about 0.05% cyclosporin A, Orange oil, and a phosphalipid.

Another embodiment comprises about 0.05% cyclosporin A, Clary sage oil, and a phosphalipid.

Another embodiment comprises about 0.05% cyclosporin A, Nutmeg oil, and a phosphalipid.

Another embodiment comprises about 0.05% cyclosporin A, Tea tree oil, and a phosphalipid.

Another embodiment comprises about 0.05% cyclosporin A, an oil having a specific gravity from 0.95 to 1.07, and phosphatidyl chloline.

Another embodiment comprises about 0.05% cyclosporin A, Anise oil, and phosphatidyl chloline.

Another embodiment comprises about 0.05% cyclosporin A, Castor oil, and phosphatidyl chloline.

Another embodiment comprises about 0.05% cyclosporin A, Clove oil, and phosphatidyl chloline.

Another embodiment comprises about 0.05% cyclosporin A, Cassia oil, and phosphatidyl chloline.

Another embodiment comprises about 0.05% cyclosporin A, Cinnamon oil, and phosphatidyl chloline.

Another embodiment comprises about 0.05% cyclosporin A, an oil having a specific gravity from 0.90 to 0.95, and phosphatidyl chloline.

Another embodiment comprises about 0.05% cyclosporin A, Almond oil, and phosphatidyl chloline.

Another embodiment comprises about 0.05% cyclosporin A, Corn oil, and phosphatidyl chloline.

Another embodiment comprises about 0.05% cyclosporin A, Arachis oil, and phosphatidyl chloline.

Another embodiment comprises about 0.05% cyclosporin A, Cottonseed oil, and phosphatidyl chloline.

Another embodiment comprises about 0.05% cyclosporin A, Safflower oil, and phosphatidyl chlorine.

Another embodiment comprises about 0.05% cyclosporin A, Maize oil, and phosphatidyl chloline.

Another embodiment comprises about 0.05% cyclosporin A, Linseed oil, and phosphatidyl chloline.

Another embodiment comprises about 0.05% cyclosporin A, Rapeseed oil, and phosphatidyl chloline.

Another embodiment comprises about 0.05% cyclosporin A, Soybean oil, and phosphatidyl chloline.

Another embodiment comprises about 0.05% cyclosporin A, Olive oil, and phosphatidyl chloline.

Another embodiment comprises about 0.05% cyclosporin A, Caraway oil, and phosphatidyl chloline.

Another embodiment comprises about 0.05% cyclosporin A, Rosemary oil, and phosphatidyl chloline.

Another embodiment comprises about 0.05% cyclosporin A, Peanut oil, and phosphatidyl chloline.

Another embodiment comprises about 0.05% cyclosporin A, Peppermint oil, and phosphatidyl chloline.

Another embodiment comprises about 0.05% cyclosporin A, Sunflower oil, and phosphatidyl chloline.

Another embodiment comprises about 0.05% cyclosporin A, Eucalyptus oil, and phosphatidyl chloline.

Another embodiment comprises about 0.05% cyclosporin A, Sesame oil, and phosphatidyl chloline.

Another embodiment comprises about 0.05% cyclosporin A, an oil having a specific gravity below 0.9, and phosphatidyl chloline.

Another embodiment comprises about 0.05% cyclosporin A, Mineral oil, and phosphatidyl chloline.

Another embodiment comprises about 0.05% cyclosporin A, Coriander oil, and phosphatidyl chloline.

Another embodiment comprises about 0.05% cyclosporin A, Lavender oil, and phosphatidyl chloline.

Another embodiment comprises about 0.05% cyclosporin A, Citronella oil, and phosphatidyl chlorine.

Another embodiment comprises about 0.05% cyclosporin A, Juniper oil, and phosphatidyl chloline.

Another embodiment comprises about 0.05% cyclosporin A, Lemon oil, and phosphatidyl chloline.

Another embodiment comprises about 0.05% cyclosporin A, Orange oil, and phosphatidyl chloline.

Another embodiment comprises about 0.05% cyclosporin A, Clary sage oil, and phosphatidyl chloline.

Another embodiment comprises about 0.05% cyclosporin A, Nutmeg oil, and phosphatidyl chlorine.

Another embodiment comprises about 0.05% cyclosporin A, Tea tree oil, and phosphatidyl chloline.

Another embodiment comprises about 0.05% cyclosporin A, an oil having a specific gravity from 0.95 to 1.07, and phosphatidyl serine.

Another embodiment comprises about 0.05% cyclosporin A, Anise oil, and phosphatidyl serine.

Another embodiment comprises about 0.05% cyclosporin A, Castor oil, and phosphatidyl serine.

Another embodiment comprises about 0.05% cyclosporin A, Clove oil, and phosphatidyl serine.

Another embodiment comprises about 0.05% cyclosporin A, Cassia oil, and phosphatidyl serine.

Another embodiment comprises about 0.05% cyclosporin A, Cinnamon oil, and phosphatidyl serine.

Another embodiment comprises about 0.05% cyclosporin A, an oil having a specific gravity from 0.90 to 0.95, and phosphatidyl serine.

Another embodiment comprises about 0.05% cyclosporin A, Almond oil, and phosphatidyl serine.

Another embodiment comprises about 0.05% cyclosporin A, Corn oil, and phosphatidyl serine.

Another embodiment comprises about 0.05% cyclosporin A, Arachis oil, and phosphatidyl serine.

Another embodiment comprises about 0.05% cyclosporin A, Cottonseed oil, and phosphatidyl serine.

Another embodiment comprises about 0.05% cyclosporin A, Safflower oil, and phosphatidyl serine.

Another embodiment comprises about 0.05% cyclosporin A, Maize oil, and phosphatidyl serine.

Another embodiment comprises about 0.05% cyclosporin A, Linseed oil, and phosphatidyl serine.

Another embodiment comprises about 0.05% cyclosporin A, Rapeseed oil, and phosphatidyl serine.

Another embodiment comprises about 0.05% cyclosporin A, Soybean oil, and phosphatidyl serine.

Another embodiment comprises about 0.05% cyclosporin A, Olive oil, and phosphatidyl serine.

Another embodiment comprises about 0.05% cyclosporin A, Caraway oil, and phosphatidyl serine.

Another embodiment comprises about 0.05% cyclosporin A, Rosemary oil, and phosphatidyl serine.

Another embodiment comprises about 0.05% cyclosporin A, Peanut oil, and phosphatidyl serine.

Another embodiment comprises about 0.05% cyclosporin A, Peppermint oil, and phosphatidyl serine.

Another embodiment comprises about 0.05% cyclosporin A, Sunflower oil, and phosphatidyl serine.

Another embodiment comprises about 0.05% cyclosporin A, Eucalyptus oil, and phosphatidyl serine.

Another embodiment comprises about 0.05% cyclosporin A, Sesame oil, and phosphatidyl serine.

Another embodiment comprises about 0.05% cyclosporin A, an oil having a specific gravity below 0.9, and phosphatidyl serine.

Another embodiment comprises about 0.05% cyclosporin A, Mineral oil, and phosphatidyl serine.

Another embodiment comprises about 0.05% cyclosporin A, Coriander oil, and phosphatidyl serine.

Another embodiment comprises about 0.05% cyclosporin A, Lavender oil, and phosphatidyl serine.

Another embodiment comprises about 0.05% cyclosporin A, Citronella oil, and phosphatidyl serine.

Another embodiment comprises about 0.05% cyclosporin A, Juniper oil, and phosphatidyl serine.

Another embodiment comprises about 0.05% cyclosporin A, Lemon oil, and phosphatidyl serine.

Another embodiment comprises about 0.05% cyclosporin A, Orange oil, and phosphatidyl serine.

Another embodiment comprises about 0.05% cyclosporin A, Clary sage oil, and phosphatidyl serine.

Another embodiment comprises about 0.05% cyclosporin A, Nutmeg oil, and phosphatidyl serine.

Another embodiment comprises about 0.05% cyclosporin A, Tea tree oil, and phosphatidyl serine.

Another embodiment comprises cyclosporin A, an alcohol, and an oil having a specific gravity from 0.95 to 1.05.

Another embodiment comprises cyclosporin A, Diglycerol®, and an oil having a specific gravity from 0.95 to 1.05.

Another embodiment comprises cyclosporin A, Hetoxide GT-80®, and an oil having a specific gravity from 0.95 to 1.05.

Another embodiment comprises cyclosporin A, Lexemul BEO®, and an oil having a specific gravity from 0.95 to 1.05.

Another embodiment comprises cyclosporin A, Polyglycerol-3®, and an oil having a specific gravity from 0.95 to 1.05.

Another embodiment comprises cyclosporin A, Redicote E Series®, and an oil having a specific gravity from 0.95 to 1.05.

Another embodiment comprises cyclosporin A, Simulsol OX 1005L®, and an oil having a specific gravity from 0.95 to 1.05.

Another embodiment comprises cyclosporin A, Stanfax 567®, and an oil having a specific gravity from 0.95 to 1.05.

Another embodiment comprises cyclosporin A, TA-1618®, and an oil having a specific gravity from 0.95 to 1.05.

Another embodiment comprises cyclosporin A, Witconol H-31A®, and an oil having a specific gravity from 0.95 to 1.05.

Another embodiment comprises cyclosporin A, Standard Div.®, and an oil having a specific gravity from 0.95 to 1.05.

Another embodiment comprises cyclosporin A, an amine oxide, and an oil having a specific gravity from 0.95 to 1.05.

Another embodiment comprises cyclosporin A, AO-405®, and an oil having a specific gravity from 0.95 to 1.05.

Another embodiment comprises cyclosporin A, AO-455®, and an oil having a specific gravity from 0.95 to 1.05.

Another embodiment comprises cyclosporin A, AO 728 Special®, and an oil having a specific gravity from 0.95 to 1.05.

Another embodiment comprises cyclosporin A, Barlox 12®, and an oil having a specific gravity from 0.95 to 1.05.

Another embodiment comprises cyclosporin A, Barlox 14®, and an oil having a specific gravity from 0.95 to 1.05.

Another embodiment comprises cyclosporin A, Burcoxide Lo®, and an oil having a specific gravity from 0.95 to 1.05.

Another embodiment comprises cyclosporin A, Caloxamine LO®, and an oil having a specific gravity from 0.95 to 1.05.

Another embodiment comprises cyclosporin A, Chemoxide CAW®, and an oil having a specific gravity from 0.95 to 1.05.

Another embodiment comprises cyclosporin A, Chemoxide LM-30®, and an oil having a specific gravity from 0.95 to 1.05.

Another embodiment comprises cyclosporin A, Chemoxide LO®, and an oil having a specific gravity from 0.95 to 1.05.

Another embodiment comprises cyclosporin A, Chemoxide MO®, and an oil having a specific gravity from 0.95 to 1.05.

Another embodiment comprises cyclosporin A, Colalux CAO-35®, and an oil having a specific gravity from 0.95 to 1.05.

Another embodiment comprises cyclosporin A, Colalux LO®, and an oil having a specific gravity from 0.95 to 1.05.

Another embodiment comprises cyclosporin A, DeMox CAPO®, and an oil having a specific gravity from 0.95 to 1.05.

Another embodiment comprises cyclosporin A, DeMox CSG-30®, and an oil having a specific gravity from 0.95 to 1.05.

Another embodiment comprises cyclosporin A, DeMox LAO®, and an oil having a specific gravity from 0.95 to 1.05.

Another embodiment comprises cyclosporin A, Emcol L®, and an oil having a specific gravity from 0.95 to 1.05.

Another embodiment comprises cyclosporin A, Empigen OB®, and an oil having a specific gravity from 0.95 to 1.05.

Another embodiment comprises cyclosporin A, Empigen OS/A®, and an oil having a specific gravity from 0.95 to 1.05.

Another embodiment comprises cyclosporin A, Foamox CDO®, and an oil having a specific gravity from 0.95 to 1.05.

Another embodiment comprises cyclosporin A, Foamox DMM®, and an oil having a specific gravity from 0.95 to 1.05.

Another embodiment comprises cyclosporin A, Foamox DMS®, and an oil having a specific gravity from 0.95 to 1.05.

Another embodiment comprises cyclosporin A, Genaminox KC®, and an oil having a specific gravity from 0.95 to 1.05.

Another embodiment comprises cyclosporin A, Genaminox LA®, and an oil having a specific gravity from 0.95 to 1.05.

Another embodiment comprises cyclosporin A, Harnofoam SAO®, and an oil having a specific gravity from 0.95 to 1.05.

Another embodiment comprises cyclosporin A, Harlox DMCD®, and an oil having a specific gravity from 0.95 to 1.05.

Another embodiment comprises cyclosporin A, Lipowax DAT®, and an oil having a specific gravity from 0.95 to 1.05.

Another embodiment comprises cyclosporin A, Lipowax PB Pastilles®, and an oil having a specific gravity from 0.95 to 1.05.

Another embodiment comprises cyclosporin A, Mackamine C8®, and an oil having a specific gravity from 0.95 to 1.05.

Another embodiment comprises cyclosporin A, Mackamine C10®, and an oil having a specific gravity from 0.95 to 1.05.

Another embodiment comprises cyclosporin A, Mackamine C14®, and an oil having a specific gravity from 0.95 to 1.05.

Another embodiment comprises cyclosporin A, Mackamine CAO®, and an oil having a specific gravity from 0.95 to 1.05.

Another embodiment comprises cyclosporin A, Mackamine CO®, and an oil having a specific gravity from 0.95 to 1.05.

Another embodiment comprises cyclosporin A, Mackamine LO®, and an oil having a specific gravity from 0.95 to 1.05.

Another embodiment comprises cyclosporin A, Mackamine O2®, and an oil having a specific gravity from 0.95 to 1.05.

Another embodiment comprises cyclosporin A, Mackamine SAO®, and an oil having a specific gravity from 0.95 to 1.05.

Another embodiment comprises cyclosporin A, Mackamine SO®, and an oil having a specific gravity from 0.95 to 1.05.

Another embodiment comprises cyclosporin A, Mazox KCAO®, and an oil having a specific gravity from 0.95 to 1.05.

Another embodiment comprises cyclosporin A, Monalac MO®, and an oil having a specific gravity from 0.95 to 1.05.

Another embodiment comprises cyclosporin A, Norfox LDA®, and an oil having a specific gravity from 0.95 to 1.05.

Another embodiment comprises cyclosporin A, Rhodamox LO®, and an oil having a specific gravity from 0.95 to 1.05.

Another embodiment comprises cyclosporin A, Schercamox C-AA®, and an oil having a specific gravity from 0.95 to 1.05.

Another embodiment comprises cyclosporin A, Schercamox DMA®, and an oil having a specific gravity from 0.95 to 1.05.

Another embodiment comprises cyclosporin A, Schercamox DML®, and an oil having a specific gravity from 0.95 to 1.05.

Another embodiment comprises cyclosporin A, Schercamox DMM®, and an oil having a specific gravity from 0.95 to 1.05.

Another embodiment comprises cyclosporin A, Schercamox DMS®, and an oil having a specific gravity from 0.95 to 1.05.

Another embodiment comprises cyclosporin A, Tegotens DO®, and an oil having a specific gravity from 0.95 to 1.05.

Another embodiment comprises cyclosporin A, Tomah AO-14-2®, and an oil having a specific gravity from 0.95 to 1.05.

Another embodiment comprises cyclosporin A, Triaminox CDO®, and an oil having a specific gravity from 0.95 to 1.05.

Another embodiment comprises cyclosporin A, a block polymer, and an oil having a specific gravity from 0.95 to 1.05.

Another embodiment comprises cyclosporin A, AL 2070®, and an oil having a specific gravity from 0.95 to 1.05.

Another embodiment comprises cyclosporin A, Antarox 17-R-2®, and an oil having a specific gravity from 0.95 to 1.05.

Another embodiment comprises cyclosporin A, Antarox 25-R-2®, and an oil having a specific gravity from 0.95 to 1.05.

Another embodiment comprises cyclosporin A, Antarox 31 -R-1®, and an oil having a specific gravity from 0.95 to 1.05.

Another embodiment comprises cyclosporin A, Antarox P-84®, and an oil having a specific gravity from 0.95 to 1.05.

Another embodiment comprises cyclosporin A, Antarox P-104/H®, and an oil having a specific gravity from 0.95 to 1.05.

Another embodiment comprises cyclosporin A, Arnox BP-Series®, and an oil having a specific gravity from 0.95 to 1.05.

Another embodiment comprises cyclosporin A, Chemonic 435®, and an oil having a specific gravity from 0.95 to 1.05.

Another embodiment comprises cyclosporin A, Chemonic D-25®, and an oil having a specific gravity from 0.95 to 1.05.

Another embodiment comprises cyclosporin A, Chemonic PL Series®, and an oil having a specific gravity from 0.95 to 1.05.

Another embodiment comprises cyclosporin A, Ethox L-121®, and an oil having a specific gravity from 0.95 to 1.05.

Another embodiment comprises cyclosporin A, Ethox L-122®, and an oil having a specific gravity from 0.95 to 1.05.

Another embodiment comprises cyclosporin A, Genapol PF-10®, and an oil having a specific gravity from 0.95 to 1.05.

Another embodiment comprises cyclosporin A, Genapol PF-20®, and an oil having a specific gravity from 0.95 to 1.05.

Another embodiment comprises cyclosporin A, Genapol PF-40A®, and an oil having a specific gravity from 0.95 to 1.05.

Another embodiment comprises cyclosporin A, Norfox 2-LF®, and an oil having a specific gravity from 0.95 to 1.05.

Another embodiment comprises cyclosporin A, Simulsol NW 342®, and an oil having a specific gravity from 0.95 to 1.05.

Another embodiment comprises cyclosporin A, T-Det BP-1®, and an oil having a specific gravity from 0.95 to 1.05.

Another embodiment comprises cyclosporin A, T-Det XD®, and an oil having a specific gravity from 0.95 to 1.05.

Another embodiment comprises cyclosporin A, T-Det XH®, and an oil having a specific gravity from 0.95 to 1.05.

Another embodiment comprises cyclosporin A, Triton CF-32®, and an oil having a specific gravity from 0.95 to 1.05.

Another embodiment comprises cyclosporin A, Witconol 171®, and an oil having a specific gravity from 0.95 to 1.05.

Another embodiment comprises cyclosporin A, Witconol 324®, and an oil having a specific gravity from 0.95 to 1.05.

Another embodiment comprises cyclosporin A, Witconol 324D®, and an oil having a specific gravity from 0.95 to 1.05.

Another embodiment comprises cyclosporin A, Witconol PD-2000®, and an oil having a specific gravity from 0.95 to 1.05.

Another embodiment comprises cyclosporin A, a Carboxylated Alcohol or Alkylphenol Ethoxylate, and an oil having a specific gravity from 0.95 to 1.05.

Another embodiment comprises cyclosporin A, Emcol CN-6®, and an oil having a specific gravity from 0.95 to 1.05.

Another embodiment comprises cyclosporin A, Ethcarb®, and an oil having a specific gravity from 0.95 to 1.05.

Another embodiment comprises cyclosporin A, Gemtex WNT-Conc®, and an oil having a specific gravity from 0.95 to 1.05.

Another embodiment comprises cyclosporin A, Incrodet TD7-C®, and an oil having a specific gravity from 0.95 to 1.05.

Another embodiment comprises cyclosporin A, Marlinat CM 105/80®, and an oil having a specific gravity from 0.95 to 1.05.

Another embodiment comprises cyclosporin A, Marlowet 1072®, and an oil having a specific gravity from 0.95 to 1.05.

Another embodiment comprises cyclosporin A, Marlowet 4530®, and an oil having a specific gravity from 0.95 to 1.05.

Another embodiment comprises cyclosporin A, Marlowet 4530 LF®, and an oil having a specific gravity from 0.95 to 1.05.

Another embodiment comprises cyclosporin A, Marlowet 4534®, and an oil having a specific gravity from 0.95 to 1.05.

Another embodiment comprises cyclosporin A, Marlowet 4538®, and an oil having a specific gravity from 0.95 to 1.05.

Another embodiment comprises cyclosporin A, Marlowet 4539®, and an oil having a specific gravity from 0.95 to 1.05.

Another embodiment comprises cyclosporin A, Marlowet 4539 LF®, and an oil having a specific gravity from 0.95 to 1.05.

Another embodiment comprises cyclosporin A, Marlowet 4541®, and an oil having a specific gravity from 0.95 to 1.05.

Another embodiment comprises cyclosporin A, Miranate LEC-80®, and an oil having a specific gravity from 0.95 to 1.05.

Another embodiment comprises cyclosporin A, Sandopan B®, and an oil having a specific gravity from 0.95 to 1.05.

Another embodiment comprises cyclosporin A, Sandopan B Modified®, and an oil having a specific gravity from 0.95 to 1.05.

Another embodiment comprises cyclosporin A, Sandopan LS-24 Gel®, and an oil having a specific gravity from 0.95 to 1.05.

Another embodiment comprises cyclosporin A, Surfine T-A®, and an oil having a specific gravity from 0.95 to 1.05.

Another embodiment comprises cyclosporin A, a Carboxylic Acid/Fatty Acid, and an oil having a specific gravity from 0.95 to 1.05.

Another embodiment comprises cyclosporin A, Colaterge RAM®, and an oil having a specific gravity from 0.95 to 1.05.

Another embodiment comprises cyclosporin A, Colatrope INC®, and an oil having a specific gravity from 0.95 to 1.05.

Another embodiment comprises cyclosporin A, Crodacid B®, and an oil having a specific gravity from 0.95 to 1.05.

Another embodiment comprises cyclosporin A, DeTrope CA-100®, and an oil having a specific gravity from 0.95 to 1.05.

Another embodiment comprises cyclosporin A, Latol MTO®, and an oil having a specific gravity from 0.95 to 1.05.

Another embodiment comprises cyclosporin A, Lumulse CC-33 K®, and an oil having a specific gravity from 0.95 to 1.05.

Another embodiment comprises cyclosporin A, Mulls 2218®, and an oil having a specific gravity from 0.95 to 1.05.

Another embodiment comprises cyclosporin A, OL-600®, and an oil having a specific gravity from 0.95 to 1.05.

Another embodiment comprises cyclosporin A, OL-800®, and an oil having a specific gravity from 0.95 to 1.05.

Another embodiment comprises cyclosporin A, R-910®, and an oil having a specific gravity from 0.95 to 1.05.

Another embodiment comprises cyclosporin A, S-210®, and an oil having a specific gravity from 0.95 to 1.05.

Another embodiment comprises cyclosporin A, Sandopan DTC Acid®, and an oil having a specific gravity from 0.95 to 1.05.

Another embodiment comprises cyclosporin Al Sandopan LS 24 N®, and an oil having a specific gravity from 0.95 to 1.05.

Another embodiment comprises cyclosporin A, Sandopan MA-18®, and an oil having a specific gravity from 0.95 to 1.05.

Another embodiment comprises cyclosporin A, an Ethoxylated Alcohol, and an oil having a specific gravity from 0.95 to 1.05.

Another embodiment comprises cyclosporin A, Adsee 799®, and an oil having a specific gravity from 0.95 to 1.05.

Another embodiment comprises cyclosporin A, Adsee 799®, and an oil having a specific gravity from 0.95 to 1.05.

Another embodiment comprises cyclosporin A, Alfonic 610-3.5®, and an oil having a specific gravity from 0.95 to 1.05.

Another embodiment comprises cyclosporin A, Alfonic 810-2®, and an oil having a specific gravity from 0.95 to 1.05.

Another embodiment comprises cyclosporin A, Alfonic 810-6®, and an oil having a specific gravity from 0.95 to 1.05.

Another embodiment comprises cyclosporin A, Alfonic 1012-3®, and an oil having a specific gravity from 0.95 to 1.05.

Another embodiment comprises cyclosporin A, Alfonic 1012-5®, and an oil having a specific gravity from 0.95 to 1.05.

Another embodiment comprises cyclosporin A, Alfonic 1216CO-1.5®, and an oil having a specific gravity from 0.95 to 1.05.

Another embodiment comprises cyclosporin A, Alfonic 1216CO-7®, and an oil having a specific gravity from 0.95 to 1.05.

Another embodiment comprises cyclosporin A, Alfonic 1412-3®, and an oil having a specific gravity from 0.95 to 1.05.

Another embodiment comprises cyclosporin A, Alfonic 1412-7®, and an oil having a specific gravity from 0.95 to 1.05.

Another embodiment comprises cyclosporin A, Arlasolve 200®, and an oil having a specific gravity from 0.95 to 1.05.

Another embodiment comprises cyclosporin A, Arlasolve 200 Liquid®, and an oil having a specific gravity from 0.95 to 1.05.

Another embodiment comprises cyclosporin A, Armix 180-C®, and an oil having a specific gravity from 0.95 to 1.05.

Another embodiment comprises cyclosporin A, Armix 183®, and an oil having a specific gravity from 0.95 to 1.05.

Another embodiment comprises cyclosporin A, Armul 2404®, and an oil having a specific gravity from 0.95 to 1.05.

Another embodiment comprises cyclosporin A, Armul 2404®, and an oil having a specific gravity from 0.95 to 1.05.

Another embodiment comprises cyclosporin A, Atlas EMJ-C®, and an oil having a specific gravity from 0.95 to 1.05.

Another embodiment comprises cyclosporin A, Atlas G-2109®, and an oil having a specific gravity from 0.95 to 1.05.

Another embodiment comprises cyclosporin A, Atlas G-3886®, and an oil having a specific gravity from 0.95 to 1.05.

Another embodiment comprises cyclosporin A, Atlas G-3890®, and an oil having a specific gravity from 0.95 to 1.05.

Another embodiment comprises cyclosporin A, Bio Soft E-200®, and an oil having a specific gravity from 0.95 to 1.05.

Another embodiment comprises cyclosporin A, Bio Soft E-300®, and an oil having a specific gravity from 0.95 to 1.05.

Another embodiment comprises cyclosporin A, Bio Soft E-400®, and an oil having a specific gravity from 0.95 to 1.05.

Another embodiment comprises cyclosporin A, Bio Soft EN 600®, and an oil having a specific gravity from 0.95 to 1.05.

Another embodiment comprises cyclosporin A, Bio Soft TD-400®, and an oil having a specific gravity from 0.95 to 1.05.

Another embodiment comprises cyclosporin A, Bio Soft TD-630®, and an oil having a specific gravity from 0.95 to 1.05.

Another embodiment comprises cyclosporin A, Brij 30®, and an oil having a specific gravity from 0.95 to 1.05.

Another embodiment comprises cyclosporin A, Brij 52®, and an oil having a specific gravity from 0.95 to 1.05.

Another embodiment comprises cyclosporin A, Brij 56®, and an oil having a specific gravity from 0.95 to 1.05.

Another embodiment comprises cyclosporin A, Brij 58®, and an oil having a specific gravity from 0.95 to 1.05.

Another embodiment comprises cyclosporin A, Brij 72®, and an oil having a specific gravity from 0.95 to 1.05.

Another embodiment comprises cyclosporin A, Brij 76®, and an oil having a specific gravity from 0.95 to 1.05.

Another embodiment comprises cyclosporin A, Brij 78®, and an oil having a specific gravity from 0.95 to 1.05.

Another embodiment comprises cyclosporin A, Brij 93®, and an oil having a specific gravity from 0.95 to 1.05.

Another embodiment comprises cyclosporin A, Brij 97®, and an oil having a specific gravity from 0.95 to 1.05.

Another embodiment comprises cyclosporin A, Brij 98®, and an oil having a specific gravity from 0.95 to 1.05.

Another embodiment comprises cyclosporin A, Brij 700®, and an oil having a specific gravity from 0.95 to 1.05.

Another embodiment comprises cyclosporin A, Brij 700 S®, and an oil having a specific gravity from 0.95 to 1.05.

Another embodiment comprises cyclosporin A, Brij 721®, and an oil having a specific gravity from 0.95 to 1.05.

Another embodiment comprises cyclosporin A, Brij 721 S®, and an oil having a specific gravity from 0.95 to 1.05.

Another embodiment comprises cyclosporin A, Burcoterge CDG®, and an oil having a specific gravity from 0.95 to 1.05.

Another embodiment comprises cyclosporin A, Canasol AT 600®, and an oil having a specific gravity from 0.95 to 1.05.

Another embodiment comprises cyclosporin A, Canasol AT 800®, and an oil having a specific gravity from 0.95 to 1.05.

Another embodiment comprises cyclosporin A, Canasol AT 1200®, and an oil having a specific gravity from 0.95 to 1.05.

Another embodiment comprises cyclosporin A, Canasol BJ 35®, and an oil having a specific gravity from 0.95 to 1.05.

Another embodiment comprises cyclosporin A, Canasol BJ 36®, and an oil having a specific gravity from 0.95 to 1.05.

Another embodiment comprises cyclosporin A, Canasol BJ 52®, and an oil having a specific gravity from 0.95 to 1.05.

Another embodiment comprises cyclosporin A, Canasol BJ 58®, and an oil having a specific gravity from 0.95 to 1.05.

Another embodiment comprises cyclosporin A, Canasol BJ 72®, and an oil having a specific gravity from 0.95 to 1.05.

Another embodiment comprises cyclosporin A, Canasol BJ 78®, and an oil having a specific gravity from 0.95 to 1.05.

Another embodiment comprises cyclosporin A, Canasol BJ 98®, and an oil having a specific gravity from 0.95 to 1.05.

Another embodiment comprises cyclosporin A, Canasol BJ 307®, and an oil having a specific gravity from 0.95 to 1.05.

Another embodiment comprises cyclosporin A, Cerfak 1400®, and an oil having a specific gravity from 0.95 to 1.05.

Another embodiment comprises cyclosporin A, Cetomacrogol 1000 BP®, and an oil having a specific gravity from 0.95 to 1.05.

Another embodiment comprises cyclosporin A, Chemonic C-2®, and an oil having a specific gravity from 0.95 to 1.05.

Another embodiment comprises cyclosporin A, Chemonic C-10®, and an oil having a specific gravity from 0.95 to 1.05.

Another embodiment comprises cyclosporin A, Chemonic C-20®, and an oil having a specific gravity from 0.95 to 1.05.

Another embodiment comprises cyclosporin A, Chemonic CT-12®, and an oil having a specific gravity from 0.95 to 1.05.

Another embodiment comprises cyclosporin A, Chemonic CT-20®, and an oil having a specific gravity from 0.95 to 1.05.

Another embodiment comprises cyclosporin A, Chemonic CT-30®, and an oil having a specific gravity from 0.95 to 1.05.

Another embodiment comprises cyclosporin A, Chemonic CT-55®, and an oil having a specific gravity from 0.95 to 1.05.

Another embodiment comprises cyclosporin A, Chemonic G-7®, and an oil having a specific gravity from 0.95 to 1.05.

Another embodiment comprises cyclosporin A, Chemonic G-26®, and an oil having a specific gravity from 0.95 to 1.05.

Another embodiment comprises cyclosporin A, Chemonic L-4®, and an oil having a specific gravity from 0.95 to 1.05.

Another embodiment comprises cyclosporin A, Chemonic L-7®, and an oil having a specific gravity from 0.95 to 1.05.

Another embodiment comprises cyclosporin A, Chemonic L-12, and an oil having a specific gravity from 0.95 to 1.05.

Another embodiment comprises cyclosporin A, Chemonic L-23®, and an oil having a specific gravity from 0.95 to 1.05.

Another embodiment comprises cyclosporin A, Chemonic O-2®, and an oil having a specific gravity from 0.95 to 1.05.

Another embodiment comprises cyclosporin A, Chemonic O-5®, and an oil having a specific gravity from 0.95 to 1.05.

Another embodiment comprises cyclosporin A, Chemonic O-10®, and an oil having a specific gravity from 0.95 to 1.05.

Another embodiment comprises cyclosporin A, Chemonic O-20®, and an oil having a specific gravity from 0.95 to 1.05.

Another embodiment comprises cyclosporin A, Chemonic S-2®, and an oil having a specific gravity from 0.95 to 1.05.

Another embodiment comprises cyclosporin A, Chemonic S-10®, and an oil having a specific gravity from 0.95 to 1.05.

Another embodiment comprises cyclosporin A, Chemonic S-20®, and an oil having a specific gravity from 0.95 to 1.05.

Another embodiment comprises cyclosporin A, Colamulse FE®, and an oil having a specific gravity from 0.95 to 1.05.

Another embodiment comprises cyclosporin A, Cremophor A 20®, and an oil having a specific gravity from 0.95 to 1.05.

Another embodiment comprises cyclosporin A, Cremophor SA 2®, and an oil having a specific gravity from 0.95 to 1.05.

Another embodiment comprises cyclosporin A, Dehydol 100®, and an oil having a specific gravity from 0.95 to 1.05.

Another embodiment comprises cyclosporin A, Dehydol O-4®, and an oil having a specific gravity from 0.95 to 1.05.

Another embodiment comprises cyclosporin A, Delonic C-18®, and an oil having a specific gravity from 0.95 to 1.05.

Another embodiment comprises cyclosporin A, DeSonic 6T®, and an oil having a specific gravity from 0.95 to 1.05.

Another embodiment comprises cyclosporin A, DeSonic 9D®, and an oil having a specific gravity from 0.95 to 1.05.

Another embodiment comprises cyclosporin A, DeSonic 9T®, and an oil having a specific gravity from 0.95 to 1.05.

Another embodiment comprises cyclosporin A, DeSonic 12D®, and an oil having a specific gravity from 0.95 to 1.05.

Another embodiment comprises cyclosporin A, DeSonic 12T®, and an oil having a specific gravity from 0.95 to 1.05.

Another embodiment comprises cyclosporin A, DeSonic 15T®, and an oil having a specific gravity from 0.95 to 1.05.

Another embodiment comprises cyclosporin A, DeSonic TDA-9®, and an oil having a specific gravity from 0.95 to 1.05.

Another embodiment comprises cyclosporin A, DeThox GLG-7®, and an oil having a specific gravity from 0.95 to 1.05.

Another embodiment comprises cyclosporin A, DeThox GLG-26®, and an oil having a specific gravity from 0.95 to 1.05.

Another embodiment comprises cyclosporin A, DeThox LA-4®, and an oil having a specific gravity from 0.95 to 1.05.

Another embodiment comprises cyclosporin A, DeThox LA-23®, and an oil having a specific gravity from 0.95 to 1.05.

Another embodiment comprises cyclosporin A, DeThox SA-80®, and an oil having a specific gravity from 0.95 to 1.05.

Another embodiment comprises cyclosporin A, Disponil O5®, and an oil having a specific gravity from 0.95 to 1.05.

Another embodiment comprises cyclosporin A, Eccoterge EO-41B®, and an oil having a specific gravity from 0.95 to 1.05.

Another embodiment comprises cyclosporin A, Empilan KA2.5/90®, and an oil having a specific gravity from 0.95 to 1.05.

Another embodiment comprises cyclosporin A, Empilan KA5/90®, and an oil having a specific gravity from 0.95 to 1.05.

Another embodiment comprises cyclosporin A, Empilan KM-20®, and an oil having a specific gravity from 0.95 to 1.05.

Another embodiment comprises cyclosporin A, Empilan KM-50®, and an oil having a specific gravity from 0.95 to 1.05.

Another embodiment comprises cyclosporin A, Empilan L-23®, and an oil having a specific gravity from 0.95 to 1.05.

Another embodiment comprises cyclosporin A, Ethylan 25-3®, and an oil having a specific gravity from 0.95 to 1.05.

Another embodiment comprises cyclosporin A, Ethylan 1204®, and an oil having a specific gravity from 0.95 to 1.05.

Another embodiment comprises cyclosporin A, Ethylan DA-4®, and an oil having a specific gravity from 0.95 to 1.05.

Another embodiment comprises cyclosporin A, Ethylan LA-230®, and an oil having a specific gravity from 0.95 to 1.05.

Another embodiment comprises cyclosporin A, Ethylan SN®, and an oil having a specific gravity from 0.95 to 1.05.

Another embodiment comprises cyclosporin A, Ethylan TD-60®, and an oil having a specific gravity from 0.95 to 1.05.

Another embodiment comprises cyclosporin A, Ethylan TD-100®, and an oil having a specific gravity from 0.95 to.1.05.

Another embodiment comprises cyclosporin A, Ethylan TD-1407®, and an oil having a specific gravity from 0.95 to 1.05.

Another embodiment comprises cyclosporin A, Eumulgin B1®, and an oil having a specific gravity from 0.95 to 1.05.

Another embodiment comprises cyclosporin A, Eumulgin B2®, and an oil having a specific gravity from 0.95 to 1.05.

Another embodiment comprises cyclosporin A, Eumulgin B3®, and an oil having a specific gravity from 0.95 to 1.05.

Another embodiment comprises cyclosporin A, Eumulgin O-10®, and an oil having a specific gravity from 0.95 to 1.05.

Another embodiment comprises cyclosporin A, Flo Mo 80/20®, and an oil having a specific gravity from 0.95 to 1.05.

Another embodiment comprises cyclosporin A, Flo Mo Low Foam®, and an oil having a specific gravity from 0.95 to 1.05.

Another embodiment comprises cyclosporin A, Forlan C-24®, and an oil having a specific gravity from 0.95 to 1.05.

Another embodiment comprises cyclosporin A, Genapol 1454®, and an oil having a specific gravity from 0.95 to 1.05.

Another embodiment comprises cyclosporin A, Genapol BA-020®, and an oil having a specific gravity from 0.95 to 1.05.

Another embodiment comprises cyclosporin A, Genapol BA-040®, and an oil having a specific gravity from 0.95 to 1.05.

Another embodiment comprises cyclosporin A, Genapol C-100®, and an oil having a specific gravity from 0.95 to 1.05.

Another embodiment comprises cyclosporin A, Genapol DA 060®, and an oil having a specific gravity from 0.95 to 1.05.

Another embodiment comprises cyclosporin A, Genapol HS 020®, and an oil having a specific gravity from 0.95 to 1.05.

Another embodiment comprises cyclosporin A, Genapol HS 200®, and an oil having a specific gravity from 0.95 to 1.05.

Another embodiment comprises cyclosporin A, Genapol ID-040®, and an oil having a specific gravity from 0.95 to 1.05.

Another embodiment comprises cyclosporin A, Genapol ID-060®, and an oil having a specific gravity from 0.95 to 1.05.

Another embodiment comprises cyclosporin A, Genapol ID-090®, and an oil having a specific gravity from 0.95 to 1.05.

Another embodiment comprises cyclosporin A, Genapol LA 010®, and an oil having a specific gravity from 0.95 to 1.05.

Another embodiment comprises cyclosporin A, Genapol LA 020®, and an oil having a specific gravity from 0.95 to 1.05.

Another embodiment comprises cyclosporin A, Genapol LA 030®, and an oil having a specific gravity from 0.95 to 1.05.

Another embodiment comprises cyclosporin A, Genapol LA 040®, and an oil having a specific gravity from 0.95 to 1.05.

Another embodiment comprises cyclosporin A, Genapol LA 050®, and an oil having a specific gravity from 0.95 to 1.05.

Another embodiment comprises cyclosporin A, Genapol LA 060®, and an oil having a specific gravity from 0.95 to 1.05.

Another embodiment comprises cyclosporin A, Genapol LA 070®, and an oil having a specific gravity from 0.95 to 1.05.

Another embodiment comprises cyclosporin A, Genapol LA 070S®, and an oil having a specific gravity from 0.95 to 1.05.

Another embodiment comprises cyclosporin A, Genapol LA 230®, and an oil having a specific gravity from 0.95 to 1.05.

Another embodiment comprises cyclosporin A, Genapol O 020®, and an oil having a specific gravity from 0.95 to 1.05.

Another embodiment comprises cyclosporin A, Genapol O 050®, and an oil having a specific gravity from 0.95 to 1.05.

Another embodiment comprises cyclosporin A, Genapol O 100®, and an oil having a specific gravity from 0.95 to 1.05.

Another embodiment comprises cyclosporin A, Genapol O 200®, and an oil having a specific gravity from 0.95 to 1.05.

Another embodiment comprises cyclosporin A, Genapol SA 030®, and an oil having a specific gravity from 0.95 to 1.05.

Another embodiment comprises cyclosporin A, Genapol SA 120®, and an oil having a specific gravity from 0.95 to 1.05.

Another embodiment comprises cyclosporin A, Genapol T-020®, and an oil having a specific gravity from 0.95 to 1.05.

Another embodiment comprises cyclosporin A, Genapol UD-030®, and an oil having a specific gravity from 0.95 to 1.05.

Another embodiment comprises cyclosporin A, Genapol UD-050®, and an oil having a specific gravity from 0.95 to 1.05.

Another embodiment comprises cyclosporin A, Genapol UD-070®, and an oil having a specific gravity from 0.95 to 1.05.

Another embodiment comprises cyclosporin A, Genapol UD-079®, and an oil having a specific gravity from 0.95 to 1.05.

Another embodiment comprises cyclosporin A, Genapol UD-080®, and an oil having a specific gravity from 0.95 to 1.05.

Another embodiment comprises cyclosporin A, Genapol UD-110®, and an oil having a specific gravity from 0.95 to 1.05.

Another embodiment comprises cyclosporin A, Genapol X 030®, and an oil having a specific gravity from 0.95 to 1.05.

Another embodiment comprises cyclosporin A, Genapol X 050®, and an oil having a specific gravity from 0.95 to 1.05.

Another embodiment comprises cyclosporin A, Genapol X 060®, and an oil having a specific gravity from 0.95 to 1.05.

Another embodiment comprises cyclosporin A, Genapol X 070®, and an oil having a specific gravity from 0.95 to 1.05.

Another embodiment comprises cyclosporin A, Genapol X 080®, and an oil having a specific gravity from 0.95 to 1.05.

Another embodiment comprises cyclosporin A, Genapol X 100®, and an oil having a specific gravity from 0.95 to 1.05.

Another embodiment comprises cyclosporin A, Genapol X 159®, and an oil having a specific gravity from 0.95 to 1.05.

Another embodiment comprises cyclosporin A, Generol 122 E5®, and an oil having a specific gravity from 0.95 to 1.05.

Another embodiment comprises cyclosporin A, Generol 122 E25®, and an oil having a specific gravity from 0.95 to 1.05.

Another embodiment comprises cyclosporin A, Hostacerin T-3®, and an oil having a specific gravity from 0.95 to 1.05.

Another embodiment comprises cyclosporin A, Iconol LF 110®, and an oil having a specific gravity from 0.95 to 1.05.

Another embodiment comprises cyclosporin A, Incropol CS-20®, and an oil having a specific gravity from 0.95 to 1.05.

Another embodiment comprises cyclosporin A, Lexemul CS-20®, and an oil having a specific gravity from 0.95 to 1.05.

Another embodiment comprises cyclosporin A, Liponic EG-1®, and an oil having a specific gravity from 0.95 to 1.05.

Another embodiment comprises cyclosporin A, Lipowax D®, and an oil having a specific gravity from 0.95 to 1.05.

Another embodiment comprises cyclosporin A, Lipowax G®, and an oil having a specific gravity from 0.95 to 1.05.

Another embodiment comprises cyclosporin A, Lipowax NI®, and an oil having a specific gravity from 0.95 to 1.05.

Another embodiment comprises cyclosporin A, Lipowax P®, and an oil having a specific gravity from 0.95 to 1.05.

Another embodiment comprises cyclosporin A, Lipowax P-31®, and an oil having a specific gravity from 0.95 to 1.05.

Another embodiment comprises cyclosporin A, Lipowax PR®, and an oil having a specific gravity from 0.95 to 1.05.

Another embodiment comprises cyclosporin A, Lumulse CS-20®, and an oil having a specific gravity from 0.95 to 1.05.

Another embodiment comprises cyclosporin A, Macol CSA-20®, and an oil having a specific gravity from 0.95 to 1.05.

Another embodiment comprises cyclosporin A, Marlox B 24/50®, and an oil having a specific gravity from 0.95 to 1.05.

Another embodiment comprises cyclosporin A, Mazawet 77®, and an oil having a specific gravity from 0.95 to 1.05.

Another embodiment comprises cyclosporin A, Norfox 1713®, and an oil having a specific gravity from 0.95 to 1.05.

Another embodiment comprises cyclosporin A, Norfox 2579®, and an oil having a specific gravity from 0.95 to 1.05.

Another embodiment comprises cyclosporin A, Norfox Lo Foam®, and an oil having a specific gravity from 0.95 to 1.05.

Another embodiment comprises cyclosporin A, Promulgen D®, and an oil having a specific gravity from 0.95 to 1.05.

Another embodiment comprises cyclosporin A, Promulgen G®, and an oil having a specific gravity from 0.95 to 1.05.

Another embodiment comprises cyclosporin A, Renex 30®, and an oil having a specific gravity from 0.95 to 1.05.

Another embodiment comprises cyclosporin A, Renex 36®, and an oil having a specific gravity from 0.95 to 1.05.

Another embodiment comprises cyclosporin A, Rhodasurf A 24®, and an oil having a specific gravity from 0.95 to 1.05.

Another embodiment comprises cyclosporin A, Rhodasurf AAE-10®, and an oil having a specific gravity from 0.95 to 1.05.

Another embodiment comprises cyclosporin A, Rhodasurf BEH-25®, and an oil having a specific gravity from 0.95 to 1.05.

Another embodiment comprises cyclosporin A, Rhodasurf BEH-40®, and an oil having a specific gravity from 0.95 to 1.05.

Another embodiment comprises cyclosporin A, Rhodasurf DA 530®, and an oil having a specific gravity from 0.95 to 1.05.

Another embodiment comprises cyclosporin A, Rhodasurf DA 630®, and an oil having a specific gravity from 0.95 to 1.05.

Another embodiment comprises cyclosporin A, Rhodasurf DA 639®, and an oil having a specific gravity from 0.95 to 1.05.

Another embodiment comprises cyclosporin A, Rhodasurf LAN-23®, and an oil having a specific gravity from 0.95 to 1.05.

Another embodiment comprises cyclosporin A, Rhodasurf ON-870®, and an oil having a specific gravity from 0.95 to 1.05.

Another embodiment comprises cyclosporin A, Rhodasurf ON-877®, and an oil having a specific gravity from 0.95 to 1.05.

Another embodiment comprises cyclosporin A, Rhodasurf TB-970 FLK®, and an oil having a specific gravity from 0.95 to 1.05.

Another embodiment comprises cyclosporin A, Ritacet-20®, and an oil having a specific gravity from 0.95 to 1.05.

Another embodiment comprises cyclosporin A, Ritachol 1000®, and an oil having a specific gravity from 0.95 to 1.05.

Another embodiment comprises cyclosporin A, Ritachol 2000®, and an oil having a specific gravity from 0.95 to 1.05.

Another embodiment comprises cyclosporin A, Ritachol 5000®, and an oil having a specific gravity from 0.95 to 1.05.

Another embodiment comprises cyclosporin A, Ritox 35®, and an oil having a specific gravity from 0.95 to 1.05.

Another embodiment comprises cyclosporin A, Surfonic DA-4®, and an oil having a specific gravity from 0.95 to 1.05.

Another embodiment comprises cyclosporin A, Surfonic DA-6®, and an oil having a specific gravity from 0.95 to 1.05.

Another embodiment comprises cyclosporin A, Surfonic L46-7®, and an oil having a specific gravity from 0.95 to 1.05.

Another embodiment comprises cyclosporin A, Surfonic POA®, and an oil having a specific gravity from 0.95 to 1.05.

Another embodiment comprises cyclosporin A, Synthrapol KB®, and an oil having a specific gravity from 0.95 to 1.05.

Another embodiment comprises cyclosporin A, Teginacid®, and an oil having a specific gravity from 0.95 to 1.05.

Another embodiment comprises cyclosporin A, Teginacid C®, and an oil having a specific gravity from 0.95 to 1.05.

Another embodiment comprises cyclosporin A, Tegotens EC 11®, and an oil having a specific gravity from 0.95 to 1.05.

Another embodiment comprises cyclosporin A, Tinegal NA®, and an oil having a specific gravity from 0.95 to 1.05.

Another embodiment comprises cyclosporin A, Tomadol 400®, and an oil having a specific gravity from 0.95 to 1.05.

Another embodiment comprises cyclosporin A, Tomadol 600®, and an oil having a specific gravity from 0.95 to 1.05.

Another embodiment comprises cyclosporin A, Tomadol 900®, and an oil having a specific gravity from 0.95 to 1.05.

Another embodiment comprises cyclosporin A, Uniperol O®, and an oil having a specific gravity from 0.95 to 1.05.

Another embodiment comprises cyclosporin A, Witconol SN Series®, and an oil having a specific gravity from 0.95 to 1.05.

Another embodiment comprises cyclosporin A, an Ethoxylated Alkylphenol, and an oil having a specific gravity from 0.95 to 1.05.

Another embodiment comprises cyclosporin A, Antarox LF-222®, and an oil having a specific gravity from 0.95 to 1.05.

Another embodiment comprises cyclosporin A, Atlox 775®, and an oil having a specific gravity from 0.95 to 1.05.

Another embodiment comprises cyclosporin A, Caloxylate N-9®, and an oil having a specific gravity from 0.95 to 1.05.

Another embodiment comprises cyclosporin A, Canasol NF-1000®, and an oil having a specific gravity from 0.95 to 1.05.

Another embodiment comprises cyclosporin A, Canasol NF-3000®, and an oil having a specific gravity from 0.95 to 1.05.

Another embodiment comprises cyclosporin A, Canasol NF-3070®, and an oil having a specific gravity from 0.95 to 1.05.

Another embodiment comprises cyclosporin A, Canasol OF 1670®, and an oil having a specific gravity from 0.95 to 1.05.

Another embodiment comprises cyclosporin A, Canasol OF 2570®, and an oil having a specific gravity from 0.95 to 1.05.

Another embodiment comprises cyclosporin A, Canasol OF 4070®, and an oil having a specific gravity from 0.95 to 1.05.

Another embodiment comprises cyclosporin A, Chemax DNP-8®, and an oil having a specific gravity from 0.95 to 1.05.

Another embodiment comprises cyclosporin A, Chemax DNP-18®, and an oil having a specific gravity from 0.95 to 1.05.

Another embodiment comprises cyclosporin A, Chemax DN P-150/50®, and an oil having a specific gravity from 0.95 to 1.05.

Another embodiment comprises cyclosporin A, DeSonic 1.5N®, and an oil having a specific gravity from 0.95 to 1.05.

Another embodiment comprises cyclosporin A, DeSonic 4N®, and an oil having a specific gravity from 0.95 to 1.05.

Another embodiment comprises cyclosporin A, DeSonic 5N®, and an oil having a specific gravity from 0.95 to 1.05.

Another embodiment comprises cyclosporin A, DeSonic 6D®, and an oil having a specific gravity from 0.95 to 1.05.

Another embodiment comprises cyclosporin A, DeSonic 6N®, and an oil having a specific gravity from 0.95 to 1.05.

Another embodiment comprises cyclosporin A, DeSonic 7N®, and an oil having a specific gravity from 0.95 to 1.05.

Another embodiment comprises cyclosporin A, DeSonic 9N®, and an oil having a specific gravity from 0.95 to 1.05.

Another embodiment comprises cyclosporin A, DeSonic 10D®, and an oil having a specific gravity from 0.95 to 1.05.

Another embodiment comprises cyclosporin A, DeSonic 11N®, and an oil having a specific gravity from 0.95 to 1.05.

Another embodiment comprises cyclosporin A, DeSonic 12N®, and an oil having a specific gravity from 0.95 to 1.05.

Another embodiment comprises cyclosporin A, DeSonic 15N®, and an oil having a specific gravity from 0.95 to 1.05.

Another embodiment comprises cyclosporin A, DeSonic 20N®, and an oil having a specific gravity from 0.95 to 1.05.

Another embodiment comprises cyclosporin A, Eccoscour RC®, and an oil having a specific gravity from 0.95 to 1.05.

Another embodiment comprises cyclosporin A, Eccoterge EO-100®, and an oil having a specific gravity from 0.95 to 1.05.

Another embodiment comprises cyclosporin A, Emulsifier 632/90%®, and an oil having a specific gravity from 0.95 to 1.05.

Another embodiment comprises cyclosporin A, Geronol AG-821®, and an oil having a specific gravity from 0.95 to 1.05.

Another embodiment comprises cyclosporin A, Gradonic N-95®, and an oil having a specific gravity from 0.95 to 1.05.

Another embodiment comprises cyclosporin A, Hetoxide NP-4®, and an oil having a specific gravity from 0.95 to 1.05.

Another embodiment comprises cyclosporin A, Hetoxide NP-30®, and an oil having a specific gravity from 0.95 to 1.05.

Another embodiment comprises cyclosporin A, Hostapal N-100®, and an oil having a specific gravity from 0.95 to 1.05.

Another embodiment comprises cyclosporin A, Hostapal N-110®, and an oil having a specific gravity from 0.95 to 1.05.

Another embodiment comprises cyclosporin A, Igepal CTA-639W®, and an oil having a specific gravity from 0.95 to 1.05.

Another embodiment comprises cyclosporin A, Igepal DAP-9®, and an oil having a specific gravity from 0.95 to 1.05.

Another embodiment comprises cyclosporin A, Igepal OD-410®, and an oil having a specific gravity from 0.95 to 1.05.

Another embodiment comprises cyclosporin A, Igepal SS-837®, and an oil having a specific gravity from 0.95 to 1.05.

Another embodiment comprises cyclosporin A, Lipocol NP-9 USP®, and an oil having a specific gravity from 0.95 to 1.05.

Another embodiment comprises cyclosporin A, Macol DNP-10®, and an oil having a specific gravity from 0.95 to 1.05.

Another embodiment comprises cyclosporin A, Marlophen NP 5®, and an oil having a specific gravity from 0.95 to 1.05.

Another embodiment comprises cyclosporin A, Marlophen P 1®, and an oil having a specific gravity from 0.95 to 1.05.

Another embodiment comprises cyclosporin A, Surfonic NB®, and an oil having a specific gravity from 0.95 to 1.05.

Another embodiment comprises cyclosporin A, Surfonic OPB-307®, and an oil having a specific gravity from 0.95 to 1.05.

Another embodiment comprises cyclosporin A, Surfonic OPB-407®, and an oil having a specific gravity from 0.95 to 1.05.

Another embodiment comprises cyclosporin A, Syn Fac 334®, and an oil having a specific gravity from 0.95 to 1.05.

Another embodiment comprises cyclosporin A, Syn Fac 8216®, and an oil having a specific gravity from 0.95 to 1.05.

Another embodiment comprises cyclosporin A, Triton N-57®, and an oil having a specific gravity from 0.95 to 1.05.

Another embodiment comprises cyclosporin A, Trycol 6956®, and an oil having a specific gravity from 0.95 to 1.05.

Another embodiment comprises cyclosporin A, Trycol 6961®, and an oil having a specific gravity from 0.95 to 1.05.

Another embodiment comprises cyclosporin A, Trycol 6964®, and an oil having a specific gravity from 0.95 to 1.05.

Another embodiment comprises cyclosporin A, Trycol 6969®, and an oil having a specific gravity from 0.95 to 1.05.

Another embodiment comprises cyclosporin A, Trycol 6974®, and an oil having a specific gravity from 0.95 to 1.05.

Another embodiment comprises cyclosporin A, Witbreak DRB-127®, and an oil having a specific gravity from 0.95 to 1.05.

Another embodiment comprises cyclosporin A, Witbreak DRB-127®, and an oil having a specific gravity from 0.95 to 1.05.

Another embodiment comprises cyclosporin A, Witconol NP Series®, and an oil having a specific gravity from 0.95 to 1.05.

Another embodiment comprises cyclosporin A, an Ethoxylated Aryl Phenol, and an oil having a specific gravity from 0.95 to 1.05.

Another embodiment comprises cyclosporin A, Soprophor BSU®, and an oil having a specific gravity from 0.95 to 1.05.

Another embodiment comprises cyclosporin A, Soprophor CY/8®, and an oil having a specific gravity from 0.95 to 1.05.

Another embodiment comprises cyclosporin A, Soprophor S/25®, and an oil having a specific gravity from 0.95 to 1.05.

Another embodiment comprises cyclosporin A, Witconol NIO®, and an oil having a specific gravity from 0.95 to 1.05.

Another embodiment comprises cyclosporin A, Witconol NIW®, and an oil having a specific gravity from 0.95 to 1.05.

Another embodiment comprises cyclosporin A, Witconol S-100®, and an oil having a specific gravity from 0.95 to 1.05.

Another embodiment comprises cyclosporin A, an Ethoxylated Fatty Acid, and an oil having a specific gravity from 0.95 to 1.05.

Another embodiment comprises cyclosporin A, Aldo PGHMS®, and an oil having a specific gravity from 0.95 to 1.05.

Another embodiment comprises cyclosporin A, Alkamuls TO-15/HR®, and an oil having a specific gravity from 0.95 to 1.05.

Another embodiment comprises cyclosporin A, Armotan AL-69-66®, and an oil having a specific gravity from 0.95 to 1.05.

Another embodiment comprises cyclosporin A, Cerasynt 840®, and an oil having a specific gravity from 0.95 to 1.05.

Another embodiment comprises cyclosporin A, Cerasynt 945®, and an oil having a specific gravity from 0.95 to 1.05.

Another embodiment comprises cyclosporin A, Crystal Inhibitor No. 5®, and an oil having a specific gravity from 0.95 to 1.05.

Another embodiment comprises cyclosporin A, DeThox Acid L-9®, and an oil having a specific gravity from 0.95 to 1.05.

Another embodiment comprises cyclosporin A, DeThox Acid S-8®, and an oil having a specific gravity from 0.95 to 1.05.

Another embodiment comprises cyclosporin A, Ethofat 242/25®, and an oil having a specific gravity from 0.95 to 1.05.

Another embodiment comprises cyclosporin A, Hydropalat 65®, and an oil having a specific gravity from 0.95 to 1.05.

Another embodiment comprises cyclosporin A, Lipo EGMS®, and an oil having a specific gravity from 0.95 to 1.05.

Another embodiment comprises cyclosporin A, Lipopeg 2 DL®, and an oil having a specific gravity from 0.95 to 1.05.

Another embodiment comprises cyclosporin A, Lipopeg 4 DL®, and an oil having a specific gravity from 0.95 to 1.05.

Another embodiment comprises cyclosporin A, Lipopeg 4-L®, and an oil having a specific gravity from 0.95 to 1.05.

Another embodiment comprises cyclosporin A, Lipopeg 39-S®, and an oil having a specific gravity from 0.95 to 1.05.

Another embodiment comprises cyclosporin A, Lipopeg 4-S®, and an oil having a specific gravity from 0.95 to 1.05.

Another embodiment comprises cyclosporin A, Lipopeg 10-S®, and an oil having a specific gravity from 0.95 to 1.05.

Another embodiment comprises cyclosporin A, Lipopeg 100-S®, and an oil having a specific gravity from 0.95 to 1.05.

Another embodiment comprises cyclosporin A, Lipopeg 6000 DS®, and an oil having a specific gravity from 0.95 to 1.05.

Another embodiment comprises cyclosporin A, Lumulse 40-L®, and an oil having a specific gravity from 0.95 to 1.05.

Another embodiment comprises cyclosporin A, Lumulse 40-S®, and an oil having a specific gravity from 0.95 to 1.05.

Another embodiment comprises cyclosporin A, Lumulse 42-L®, and an oil having a specific gravity from 0.95 to 1.05.

Another embodiment comprises cyclosporin A, Lumulse 42-S®, and an oil having a specific gravity from 0.95 to 1.05.

Another embodiment comprises cyclosporin A, Lumulse 100-S®, and an oil having a specific gravity from 0.95 to 1.05.

Another embodiment comprises cyclosporin A, Lumulse 602-S®, and an oil having a specific gravity from 0.95 to 1.05.

Another embodiment comprises cyclosporin A, Magrabar PGE-20-0®, and an oil having a specific gravity from 0.95 to 1.05.

Another embodiment comprises cyclosporin A, Magrabar PGE-20L®, and an oil having a specific gravity from 0.95 to 1.05.

Another embodiment comprises cyclosporin A, Magrabar PGE-20T®, and an oil having a specific gravity from 0.95 to 1.05.

Another embodiment comprises cyclosporin A, Magrabar PGE-22-0®, and an oil having a specific gravity from 0.95 to 1.05.

Another embodiment comprises cyclosporin A, Magrabar PGE-22L®, and an oil having a specific gravity from 0.95 to 1.05.

Another embodiment comprises cyclosporin A, Magrabar PGE-22T®, and an oil having a specific gravity from 0.95 to 1.05.

Another embodiment comprises cyclosporin A, Magrabar PGE-40-0®, and an oil having a specific gravity from 0.95 to 1.05.

Another embodiment comprises cyclosporin A, Magrabar PGE-40L®, and an oil having a specific gravity from 0.95 to 1.05.

Another embodiment comprises cyclosporin A, Magrabar PGE-40T®, and an oil having a specific gravity from 0.95 to 1.05.

Another embodiment comprises cyclosporin A, Magrabar PGE-42-0®, and an oil having a specific gravity from 0.95 to 1.05.

Another embodiment comprises cyclosporin A, Magrabar PGE-42L®, and an oil having a specific gravity from 0.95 to 1.05.

Another embodiment comprises cyclosporin A, Magrabar PGE-42T®, and an oil having a specific gravity from 0.95 to 1.05.

Another embodiment comprises cyclosporin A, Magrabar PGE-60-0®, and an oil having a specific gravity from 0.95 to 1.05.

Another embodiment comprises cyclosporin A, Magrabar PGE-60L®, and an oil having a specific gravity from 0.95 to 1.05.

Another embodiment comprises cyclosporin A, Magrabar PGE-60T®, and an oil having a specific gravity from 0.95 to 1.05.

Another embodiment comprises cyclosporin A, Magrabar PGE-62-0®, and an oil having a specific gravity from 0.95 to 1.05.

Another embodiment comprises cyclosporin A, Magrabar PGE-62L®, and an oil having a specific gravity from 0.95 to 1.05.

Another embodiment comprises cyclosporin A, Magrabar PGE-62T®, and an oil having a specific gravity from 0.95 to 1.05.

Another embodiment comprises cyclosporin A, Mapeg S-40K®, and an oil having a specific gravity from 0.95 to 1.05.

Another embodiment comprises cyclosporin A, Marlowet OTS®, and an oil having a specific gravity from 0.95 to 1.05.

Another embodiment comprises cyclosporin A, Naturechem PGR®, and an oil having a specific gravity from 0.95 to 1.05.

Another embodiment comprises cyclosporin A, PG No. 4®, and an oil having a specific gravity from 0.95 to 1.05.

Another embodiment comprises cyclosporin A, Renex 20®, and an oil having a specific gravity from 0.95 to 1.05.

Another embodiment comprises cyclosporin A, Ritox 52®, and an oil having a specific gravity from 0.95 to 1.05.

Another embodiment comprises cyclosporin A, Ritox 53®, and an oil having a specific gravity from 0.95 to 1.05.

Another embodiment comprises cyclosporin A, Ritox 59®, and an oil having a specific gravity from 0.95 to 1.05.

Another embodiment comprises cyclosporin A, Surfax 891 6/A®, and an oil having a specific gravity from 0.95 to 1.05.

Another embodiment comprises cyclosporin A, Tego Acid S 40 P®, and an oil having a specific gravity from 0.95 to 1.05.

Another embodiment comprises cyclosporin A, Tego Acid S 100 P®, and an oil having a specific gravity from 0.95 to 1.05.

Another embodiment comprises cyclosporin A, Tween 20®, and an oil having a specific gravity from 0.95 to 1.05.

Another embodiment comprises cyclosporin A, Volpo 131®, and an oil having a specific gravity from 0.95 to 1.05.

Another embodiment comprises cyclosporin A, an Ethoxylated Fatty Ester or Oil, and an oil having a specific gravity from 0.95 to 1.05.

Another embodiment comprises cyclosporin A, Acconon 6-C10®, and an oil having a specific gravity from 0.95 to 1.05.

Another embodiment comprises cyclosporin A, Acconon CC-6®, and an oil having a specific gravity from 0.95 to 1.05.

Another embodiment comprises cyclosporin A, Acconon CO-7®, and an oil having a specific gravity from 0.95 to 1.05.

Another embodiment comprises cyclosporin A, Aldosperse 40/60 FG®, and an oil having a specific gravity from 0.95 to 1.05.

Another embodiment comprises cyclosporin A, Aldosperse ML-23®, and an oil having a specific gravity from 0.95 to 1.05.

Another embodiment comprises cyclosporin A, Aldosperse MS-20 FG®, and an oil having a specific gravity from 0.95 to 1.05.

Another embodiment comprises cyclosporin A, Alkamuls EL-620®, and an oil having a specific gravity from 0.95 to 1.05.

Another embodiment comprises cyclosporin A, Alkamuls EL-719®, and an oil having a specific gravity from 0.95 to 1.05.

Another embodiment comprises cyclosporin A, Alkamuls EL-985®, and an oil having a specific gravity from 0.95 to 1.05.

Another embodiment comprises cyclosporin A, Arlatone G®, and an oil having a specific gravity from 0.95 to 1.05.

Another embodiment comprises cyclosporin A, Arlatone T®, and an oil having a specific gravity from 0.95 to 1.05.

Another embodiment comprises cyclosporin A, Atlas G-1045A®, and an oil having a specific gravity from 0.95 to 1.05.

Another embodiment comprises cyclosporin A, Atlas G-1086®, and an oil having a specific gravity from 0.95 to 1.05.

Another embodiment comprises cyclosporin A, Atlas G-1087®, and an oil having a specific gravity from 0.95 to 1.05.

Another embodiment comprises cyclosporin A, Atlas G-1089®, and an oil having a specific gravity from 0.95 to 1.05.

Another embodiment comprises cyclosporin A, Atlas G-1096®, and an oil having a specific gravity from 0.95 to 1.05.

Another embodiment comprises cyclosporin A, Atlas G-1292®, and an oil having a specific gravity from 0.95 to 1.05.

Another embodiment comprises cyclosporin A, Atlas G-1293®, and an oil having a specific gravity from 0.95 to 1.05.

Another embodiment comprises cyclosporin A, Atlas G-1300®, and an oil having a specific gravity from 0.95 to 1.05.

Another embodiment comprises cyclosporin A, Atlas G-7076®, and an oil having a specific gravity from 0.95 to 1.05.

Another embodiment comprises cyclosporin A, Capmul EMG®, and an oil having a specific gravity from 0.95 to 1.05.

Another embodiment comprises cyclosporin A, Chemonic CO-40®, and an oil having a specific gravity from 0.95 to 1.05.

Another embodiment comprises cyclosporin A, Chemonic LI-3®, and an oil having a specific gravity from 0.95 to 1.05.

Another embodiment comprises cyclosporin A, Chemonic LI-7®, and an oil having a specific gravity from 0.95 to 1.05.

Another embodiment comprises cyclosporin A, Cirrasol GM®, and an oil having a specific gravity from 0.95 to 1.05.

Another embodiment comprises cyclosporin A, Cremophor CO 40®, and an oil having a specific gravity from 0.95 to 1.05.

Another embodiment comprises cyclosporin A, Cremophor CO 410®, and an oil having a specific gravity from 0.95 to 1.05.

Another embodiment comprises cyclosporin A, Cremophor EL®, and an oil having a specific gravity from 0.95 to 1.05.

Another embodiment comprises cyclosporin A, Cremophor GC7®, and an oil having a specific gravity from 0.95 to 1.05.

Another embodiment comprises cyclosporin A, Cremophor RH-40®, and an oil having a specific gravity from 0.95 to 1.05.

Another embodiment comprises cyclosporin A, Crovol A-40®, and an oil having a specific gravity from 0.95 to 1.05.

Another embodiment comprises cyclosporin A, Crovol A-70®, and an oil having a specific gravity from 0.95 to 1.05.

Another embodiment comprises cyclosporin A, Crovol M-70®, and an oil having a specific gravity from 0.95 to 1.05.

Another embodiment comprises cyclosporin A, Crovol PK-70®, and an oil having a specific gravity from 0.95 to 1.05.

Another embodiment comprises cyclosporin A, Cutina E-24®, and an oil having a specific gravity from 0.95 to 1.05.

Another embodiment comprises cyclosporin A, Dacospin 12-R®, and an oil having a specific gravity from 0.95 to 1.05.

Another embodiment comprises cyclosporin A, Dehymuls HRE-7®, and an oil having a specific gravity from 0.95 to 1.05.

Another embodiment comprises cyclosporin A, DeSonic 30C®, and an oil having a specific gravity from 0.95 to 1.05.

Another embodiment comprises cyclosporin A, DeSonic 36C®, and an oil having a specific gravity from 0.95 to 1.05.

Another embodiment comprises cyclosporin A, DeSonic 40C®, and an oil having a specific gravity from 0.95 to 1.05.

Another embodiment comprises cyclosporin A, Durfax 60®, and an oil having a specific gravity from 0.95 to 1.05.

Another embodiment comprises cyclosporin A, Durfax 65®, and an oil having a specific gravity from 0.95 to 1.05.

Another embodiment comprises cyclosporin A, Durfax 80®, and an oil having a specific gravity from 0.95 to 1.05.

Another embodiment comprises cyclosporin A, Durfax EOM®, and an oil having a specific gravity from 0.95 to 1.05.

Another embodiment comprises cyclosporin A, Eccoterge NF-2®, and an oil having a specific gravity from 0.95 to 1.05.

Another embodiment comprises cyclosporin A, Emulpon CO-360®, and an oil having a specific gravity from 0.95 to 1.05.

Another embodiment comprises cyclosporin A, Emulpon CO-550®, and an oil having a specific gravity from 0.95 to 1.05.

Another embodiment comprises cyclosporin A, Emulsogen EL®, and an oil having a specific gravity from 0.95 to 1.05.

Another embodiment comprises cyclosporin A, Emulsogen HCO 040®, and an oil having a specific gravity from 0.95 to 1.05.

Another embodiment comprises cyclosporin A, Emulsogen HCO 060®, and an oil having a specific gravity from 0.95 to 1.05.

Another embodiment comprises cyclosporin A, Emulsynt 1055®, and an oil having a specific gravity from 0.95 to 1.05.

Another embodiment comprises cyclosporin A, Ethox 3095®, and an oil having a specific gravity from 0.95 to 1.05.

Another embodiment comprises cyclosporin A, Eumulgin RO-40®, and an oil having a specific gravity from 0.95 to 1.05.

Another embodiment comprises cyclosporin A, Genapol G-260®, and an oil having a specific gravity from 0.95 to 1.05.

Another embodiment comprises cyclosporin A, Glycosperse L-20®, and an oil having a specific gravity from 0.95 to 1.05.

Another embodiment comprises cyclosporin A, Glycosperse O-5®, and an oil having a specific gravity from 0.95 to 1.05.

Another embodiment comprises cyclosporin A, Glycosperse O-20®, and an oil having a specific gravity from 0.95 to 1.05.

Another embodiment comprises cyclosporin A, Glycosperse O-20 FG®, and an oil having a specific gravity from 0.95 to 1.05.

Another embodiment comprises cyclosporin A, Glycosperse S-20®, and an oil having a specific gravity from 0.95 to 1.05.

Another embodiment comprises cyclosporin A, Glycosperse S-20 FG®, and an oil having a specific gravity from 0.95 to 1.05.

Another embodiment comprises cyclosporin A, Glycosperse TS-20®, and an oil having a specific gravity from 0.95 to 1.05.

Another embodiment comprises cyclosporin A, Glycosperse TS-20 FG®, and an oil having a specific gravity from 0.95 to 1.05.

Another embodiment comprises cyclosporin A, Hetan SL®, and an oil having a specific gravity from 0.95 to 1.05.

Another embodiment comprises cyclosporin A, Hetan SO®, and an oil having a specific gravity from 0.95 to 1.05.

Another embodiment comprises cyclosporin A, Hetan SS®, and an oil having a specific gravity from 0.95 to 1.05.

Another embodiment comprises cyclosporin A, Hetoxide C-2®, and an oil having a specific gravity from 0.95 to 1.05.

Another embodiment comprises cyclosporin A, Hetoxide C-9®, and an oil having a specific gravity from 0.95 to 1.05.

Another embodiment comprises cyclosporin A, Hetoxide C-15®, and an oil having a specific gravity from 0.95 to 1.05.

Another embodiment comprises cyclosporin A, Hetoxide C-25®, and an oil having a specific gravity from 0.95 to 1.05.

Another embodiment comprises cyclosporin A, Hetoxide C-40®, and an oil having a specific gravity from 0.95 to 1.05.

Another embodiment comprises cyclosporin A, Hetoxide C-200®, and an oil having a specific gravity from 0.95 to 1.05.

Another embodiment comprises cyclosporin A, Hetoxide C-200-50%®, and an oil having a specific gravity from 0.95 to 1.05.

Another embodiment comprises cyclosporin A, Hetoxide GC-30®, and an oil having a specific gravity from 0.95 to 1.05.

Another embodiment comprises cyclosporin A, Hetoxide HC-60®, and an oil having a specific gravity from 0.95 to 1.05.

Another embodiment comprises cyclosporin A, Ice No. 2®, and an oil having a specific gravity from 0.95 to 1.05.

Another embodiment comprises cyclosporin A, Incrocas 30/40®, and an oil having a specific gravity from 0.95 to 1.05.

Another embodiment comprises cyclosporin A, Lexol EC®, and an oil having a specific gravity from 0.95 to 1.05.

Another embodiment comprises cyclosporin A, Lexol EO®, and an oil having a specific gravity from 0.95 to 1.05.

Another embodiment comprises cyclosporin A, Lipocol HCO-40®, and an oil having a specific gravity from 0.95 to 1.05.

Another embodiment comprises cyclosporin A, Lipocol HCO-60®, and an oil having a specific gravity from 0.95 to 1.05.

Another embodiment comprises cyclosporin A, Lipocol O-3 Special®, and an oil having a specific gravity from 0.95 to 1.05.

Another embodiment comprises cyclosporin A, Lipopeg 2-L®, and an oil having a specific gravity from 0.95 to 1.05.

Another embodiment comprises cyclosporin A, Lipopeg 4-DO®, and an oil having a specific gravity from 0.95 to 1.05.

Another embodiment comprises cyclosporin A, Lipopeg 4-DS®, and an oil having a specific gravity from 0.95 to 1.05.

Another embodiment comprises cyclosporin A, Lipovol GTB®, and an oil having a specific gravity from 0.95 to 1.05.

Another embodiment comprises cyclosporin A, Lonzest SML-20®, and an oil having a specific gravity from 0.95 to 1.05.

Another embodiment comprises cyclosporin A, Lonzest SMO-20®, and an oil having a specific gravity from 0.95 to 1.05.

Another embodiment comprises cyclosporin A, Lonzest SMS-20®, and an oil having a specific gravity from 0.95 to 1.05.

Another embodiment comprises cyclosporin A, Lonzest STO-20®, and an oil having a specific gravity from 0.95 to 1.05.

Another embodiment comprises cyclosporin A, Lonzest STS-20®, and an oil having a specific gravity from 0.95 to 1.05.

Another embodiment comprises cyclosporin A, Lumulse GR-40®, and an oil having a specific gravity from 0.95 to 1.05.

Another embodiment comprises cyclosporin A, Lumulse GRH-40®, and an oil having a specific gravity from 0.95 to 1.05.

Another embodiment comprises cyclosporin A, Lumulse POE (7) GML®, and an oil having a specific gravity from 0.95 to 1.05.

Another embodiment comprises cyclosporin A, Lumulse POE (12) Glyc®, and an oil having a specific gravity from 0.95 to 1.05.

Another embodiment comprises cyclosporin A, Lumulse POE (40) MS KP®, and an oil having a specific gravity from 0.95 to 1.05.

Another embodiment comprises cyclosporin A, Marlowet 4750®, and an oil having a specific gravity from 0.95 to 1.05.

Another embodiment comprises cyclosporin A, Marlowet LVS®, and an oil having a specific gravity from 0.95 to 1.05.

Another embodiment comprises cyclosporin A, Marlowet R 11®, and an oil having a specific gravity from 0.95 to 1.05.

Another embodiment comprises cyclosporin A, Marlowet R 40®, and an oil having a specific gravity from 0.95 to 1.05.

Another embodiment comprises cyclosporin A, Mazol 80 MGK®, and an oil having a specific gravity from 0.95 to 1.05.

Another embodiment comprises cyclosporin A, Nonionic Emulsifier T-9®, and an oil having a specific gravity from 0.95 to 1.05.

Another embodiment comprises cyclosporin A, Oronal LCG®, and an oil having a specific gravity from 0.95 to 1.05.

Another embodiment comprises cyclosporin A, Polyderm PPI-CO-200®, and an oil having a specific gravity from 0.95 to 1.05.

Another embodiment comprises cyclosporin A, Polyderm PPI-CO-40®, and an oil having a specific gravity from 0.95 to 1.05.

Another embodiment comprises cyclosporin A, Rewoderm LI 520-70®, and an oil having a specific gravity from 0.95 to 1.05.

Another embodiment comprises cyclosporin A, Ritapeg 150 DS®, and an oil having a specific gravity from 0.95 to 1.05.

Another embodiment comprises cyclosporin A, Softigen 767®, and an oil having a specific gravity from 0.95 to 1.05.

Another embodiment comprises cyclosporin A, Surfactol 318®, and an oil having a specific gravity from 0.95 to 1.05.

Another embodiment comprises cyclosporin A, Surfactol 365®, and an oil having a specific gravity from 0.95 to 1.05.

Another embodiment comprises cyclosporin A, Syn Lube 107®, and an oil having a specific gravity from 0.95 to 1.05.

Another embodiment comprises cyclosporin A, Syn Lube 728®, and an oil having a specific gravity from 0.95 to 1.05.

Another embodiment comprises cyclosporin A, Syn Lube 1632H®, and an oil having a specific gravity from 0.95 to 1.05.

Another embodiment comprises cyclosporin A, Syn Lube 6277-A®, and an oil having a specific gravity from 0.95 to 1.05.

Another embodiment comprises cyclosporin A, T-Det C-20®, and an oil having a specific gravity from 0.95 to 1.05.

Another embodiment comprises cyclosporin A, T-Det C-40®, and an oil having a specific gravity from 0.95 to 1.05.

Another embodiment comprises cyclosporin A, Tally 100 Plus®, and an oil having a specific gravity from 0.95 to 1.05.

Another embodiment comprises cyclosporin A, Uniperol EL®, and an oil having a specific gravity from 0.95 to 1.05.

Another embodiment comprises cyclosporin A, a Fatty Ester, and an oil having a specific gravity from 0.95 to 1.05.

Another embodiment comprises cyclosporin A, Actralube-Syn 147®, and an oil having a specific gravity from 0.95 to 1.05.

Another embodiment comprises cyclosporin A, Atlas G-1556®, and an oil having a specific gravity from 0.95 to 1.05.

Another embodiment comprises cyclosporin A, Atlas G-1564®, and an oil having a specific gravity from 0.95 to 1.05.

Another embodiment comprises cyclosporin A, Atlasol Base Oil S®, and an oil having a specific gravity from 0.95 to 1.05.

Another embodiment comprises cyclosporin A, Base ML®, and an oil having a specific gravity from 0.95 to 1.05.

Another embodiment comprises cyclosporin A, Base MT®, and an oil having a specific gravity from 0.95 to 1.05.

Another embodiment comprises cyclosporin A, Cerasynt 303®, and an oil having a specific gravity from 0.95 to 1.05.

Another embodiment comprises cyclosporin A, Dermol 1012®, and an oil having a specific gravity from 0.95 to 1.05.

Another embodiment comprises cyclosporin A, Kemester 4000®, and an oil having a specific gravity from 0.95 to 1.05.

Another embodiment comprises cyclosporin A, Lactipol S®, and an oil having a specific gravity from 0.95 to 1.05.

Another embodiment comprises cyclosporin A, Magrabar PGO®, and an oil having a specific gravity from 0.95 to 1.05.

Another embodiment comprises cyclosporin A, Mayco Base BFO®, and an oil having a specific gravity from 0.95 to 1.05.

Another embodiment comprises cyclosporin A, Methyl Linoleate®, and an oil having a specific gravity from 0.95 to 1.05.

Another embodiment comprises cyclosporin A, Pationic 122A®, and an oil having a specific gravity from 0.95 to 1.05.

Another embodiment comprises cyclosporin A, Pationic 138C®, and an oil having a specific gravity from 0.95 to 1.05.

Another embodiment comprises cyclosporin A, Pationic CSL®, and an oil having a specific gravity from 0.95 to 1.05.

Another embodiment comprises cyclosporin A, Pationic ISL®, and an oil having a specific gravity from 0.95 to 1.05.

Another embodiment comprises cyclosporin A, Pationic SBL®, and an oil having a specific gravity from 0.95 to 1.05.

Another embodiment comprises cyclosporin A, Pationic SSL®, and an oil having a specific gravity from 0.95 to 1.05.

Another embodiment comprises cyclosporin A, Ritasol®, and an oil having a specific gravity from 0.95 to 1.05.

Another embodiment comprises cyclosporin A, Tego Alkanol CS 20®, and an oil having a specific gravity from 0.95 to 1.05.

Another embodiment comprises cyclosporin A, Tego Alkanol L23 P®, and an oil having a specific gravity from 0.95 to 1.05.

Another embodiment comprises cyclosporin A, Tego Alkanol S2®, and an oil having a specific gravity from 0.95 to 1.05.

Another embodiment comprises cyclosporin A, Tego Alkanol S20 P®, and an oil having a specific gravity from 0.95 to 1.05.

Another embodiment comprises cyclosporin A, Triemulsifier 600 MS®, and an oil having a specific gravity from 0.95 to 1.05.

Another embodiment comprises cyclosporin A, a Glycerol Esters, and an oil having a specific gravity from 0.95 to 1.05.

Another embodiment comprises cyclosporin A, Agro #9 Wint SBO®, and an oil having a specific gravity from 0.95 to 1.05.

Another embodiment comprises cyclosporin A, Ahcovel Base 700®, and an oil having a specific gravity from 0.95 to 1.05.

Another embodiment comprises cyclosporin A, Aldo HMS FG®, and an oil having a specific gravity from 0.95 to 1.05.

Another embodiment comprises cyclosporin A, Aldo MLD®, and an oil having a specific gravity from 0.95 to 1.05.

Another embodiment comprises cyclosporin A, Aldo MLD FG®, and an oil having a specific gravity from 0.95 to 1.05.

Another embodiment comprises cyclosporin A, Aldo MO FG®, and an oil having a specific gravity from 0.95 to 1.05.

Another embodiment comprises cyclosporin A, Aldo MS®, and an oil having a specific gravity from 0.95 to 1.05.

Another embodiment comprises cyclosporin A, Aldo MS FG®, and an oil having a specific gravity from 0.95 to 1.05.

Another embodiment comprises cyclosporin A, Aldo MS LG FG®, and an oil having a specific gravity from 0.95 to 1.05.

Another embodiment comprises cyclosporin A, Aldo MSD®, and an oil having a specific gravity from 0.95 to 1.05.

Another embodiment comprises cyclosporin A, Aldo MSD FG®, and an oil having a specific gravity from 0.95 to 1.05.

Another embodiment comprises cyclosporin A, Aldosperse O-20 FG®, and an oil having a specific gravity from 0.95 to 1.05.

Another embodiment comprises cyclosporin A, Aldosperse TS-20 FG®, and an oil having a specific gravity from 0.95 to 1.05.

Another embodiment comprises cyclosporin A, Aldosperse TS-40 FG®, and an oil having a specific gravity from 0.95 to 1.05.

Another embodiment comprises cyclosporin A, Arlacel 165®, and an oil having a specific gravity from 0.95 to 1.05.

Another embodiment comprises cyclosporin A, Arlacel 186®, and an oil having a specific gravity from 0.95 to 1.05.

Another embodiment comprises cyclosporin A, Capmul GMO®, and an oil having a specific gravity from 0.95 to 1.05.

Another embodiment comprises cyclosporin A, Capmul GMS®, and an oil having a specific gravity from 0.95 to 1.05.

Another embodiment comprises cyclosporin A, Caprol 3GO®, and an oil having a specific gravity from 0.95 to 1.05.

Another embodiment comprises cyclosporin A, Caprol 3GVS®, and an oil having a specific gravity from 0.95 to 1.05.

Another embodiment comprises cyclosporin A, Caprol 6G2S®, and an oil having a specific gravity from 0.95 to 1.05.

Another embodiment comprises cyclosporin A, Caprol 10G40®, and an oil having a specific gravity from 0.95 to 1.05.

Another embodiment comprises cyclosporin A, Caprol 10G100®, and an oil having a specific gravity from 0.95 to 1.05.

Another embodiment comprises cyclosporin A, Caprol ET®, and an oil having a specific gravity from 0.95 to 1.05.

Another embodiment comprises cyclosporin A, Caprol PGE860®, and an oil having a specific gravity from 0.95 to 1.05.

Another embodiment comprises cyclosporin A, Cerasynt 945®, and an oil having a specific gravity from 0.95 to 1.05.

Another embodiment comprises cyclosporin A, Cerasynt GMS®, and an oil having a specific gravity from 0.95 to 1.05.

Another embodiment comprises cyclosporin A, Cerasynt Q®, and an oil having a specific gravity from 0.95 to 1.05.

Another embodiment comprises cyclosporin A, Cerasynt SD®, and an oil having a specific gravity from 0.95 to 1.05.

Another embodiment comprises cyclosporin A, Cerasynt WM®, and an oil having a specific gravity from 0.95 to 1.05.

Another embodiment comprises cyclosporin A, Chemsperse 14®, and an oil having a specific gravity from 0.95 to 1.05.

Another embodiment comprises cyclosporin A, Cremophor GO-32®, and an oil having a specific gravity from 0.95 to 1.05.

Another embodiment comprises cyclosporin A, Cremophor GS11®, and an oil having a specific gravity from 0.95 to 1.05.

Another embodiment comprises cyclosporin A, Cremophor GS-32®, and an oil having a specific gravity from 0.95 to 1.05.

Another embodiment comprises cyclosporin A, Cutina KD-16®, and an oil having a specific gravity from 0.95 to 1.05.

Another embodiment comprises cyclosporin A, Dehymuls PGPH®, and an oil having a specific gravity from 0.95 to 1.05.

Another embodiment comprises cyclosporin A, Dermol DGDIS®, and an oil having a specific gravity from 0.95 to 1.05.

Another embodiment comprises cyclosporin A, Dermol DGMIS®, and an oil having a specific gravity from 0.95 to 1.05.

Another embodiment comprises cyclosporin A, Dermol G-76®, and an oil having a specific gravity from 0.95 to 1.05.

Another embodiment comprises cyclosporin A, Dermol G-7DI®, and an oil having a specific gravity from 0.95 to 1.05.

Another embodiment comprises cyclosporin A, Dermol NGDI®, and an oil having a specific gravity from 0.95 to 1.05.

Another embodiment comprises cyclosporin A, Dermolan GLH®, and an oil having a specific gravity from 0.95 to 1.05.

Another embodiment comprises cyclosporin A, Drewmulse GMO®, and an oil having a specific gravity from 0.95 to 1.05.

Another embodiment comprises cyclosporin A, Drewpol 3-5-M®, and an oil having a specific gravity from 0.95 to 1.05.

Another embodiment comprises cyclosporin A, Durlac 100 W®, and an oil having a specific gravity from 0.95 to 1.05.

Another embodiment comprises cyclosporin A, Dur-Lo®, and an oil having a specific gravity from 0.95 to 1.05.

Another embodiment comprises cyclosporin A, Dynasan 118®, and an oil having a specific gravity from 0.95 to 1.05.

Another embodiment comprises cyclosporin A, EC-25®, and an oil having a specific gravity from 0.95 to 1.05.

Another embodiment comprises cyclosporin A, EM 40®, and an oil having a specific gravity from 0.95 to 1.05.

Another embodiment comprises cyclosporin A, Emerest 2400®, and an oil having a specific gravity from 0.95 to 1.05.

Another embodiment comprises cyclosporin A, Emerest 2452®, and an oil having a specific gravity from 0.95 to 1.05.

Another embodiment comprises cyclosporin A, Empilan G-26®, and an oil having a specific gravity from 0.95 to 1.05.

Another embodiment comprises cyclosporin A, Genapol TSM®, and an oil having a specific gravity from 0.95 to 1.05.

Another embodiment comprises cyclosporin A, Hostacerin DGI®, and an oil having a specific gravity from 0.95 to 1.05.

Another embodiment comprises cyclosporin A, Hostacerin DGL®, and an oil having a specific gravity from 0.95 to 1.05.

Another embodiment comprises cyclosporin A, Hostacerin DGMS®, and an oil having a specific gravity from 0.95 to 1.05.

Another embodiment comprises cyclosporin A, Hostacerin DGSB®, and an oil having a specific gravity from 0.95 to 1.05.

Another embodiment comprises cyclosporin A, Ice No. 2®, and an oil having a specific gravity from 0.95 to 1.05.

Another embodiment comprises cyclosporin A, Imwitor 742®, and an oil having a specific gravity from 0.95 to 1.05.

Another embodiment comprises cyclosporin A, Imwitor 780 K®, and an oil having a specific gravity from 0.95 to 1.05.

Another embodiment comprises cyclosporin A, Imwitor 960 Flakes®, and an oil having a specific gravity from 0.95 to 1.05.

Another embodiment comprises cyclosporin A, Isolan GI 34®, and an oil having a specific gravity from 0.95 to 1.05.

Another embodiment comprises cyclosporin A, Isolan GO 33®, and an oil having a specific gravity from 0.95 to 1.05.

Another embodiment comprises cyclosporin A, Kemester 1000®, and an oil having a specific gravity from 0.95 to 1.05.

Another embodiment comprises cyclosporin A, Kemester 2000®, and an oil having a specific gravity from 0.95 to 1.05.

Another embodiment comprises cyclosporin A, Kemester 2000®, and an oil having a specific gravity from 0.95 to 1.05.

Another embodiment comprises cyclosporin A, Kemester 6000SE®, and an oil having a specific gravity from 0.95 to 1.05.

Another embodiment comprises cyclosporin A, Lamecreme DGE 18®, and an oil having a specific gravity from 0.95 to 1.05.

Another embodiment comprises cyclosporin A, Lexemul 515®, and an oil having a specific gravity from 0.95 to 1.05.

Another embodiment comprises cyclosporin A, Lexemul 561®, and an oil having a specific gravity from 0.95 to 1.05.

Another embodiment comprises cyclosporin A, Lexemul AR®, and an oil having a specific gravity from 0.95 to 1.05.

Another embodiment comprises cyclosporin A, Lexemul AS®, and an oil having a specific gravity from 0.95 to 1.05.

Another embodiment comprises cyclosporin A, Lexemul GDL®, and an oil having a specific gravity from 0.95 to 1.05.

Another embodiment comprises cyclosporin A, Lexemul T®, and an oil having a specific gravity from 0.95 to 1.05.

Another embodiment comprises cyclosporin A, Lipomulse 165®, and an oil having a specific gravity from 0.95 to 1.05.

Another embodiment comprises cyclosporin A, Lumulse GML K®, and an oil having a specific gravity from 0.95 to 1.05.

Another embodiment comprises cyclosporin A, Lumulse GMO K®, and an oil having a specific gravity from 0.95 to 1.05.

Another embodiment comprises cyclosporin A, Lumulse GMR K®, and an oil having a specific gravity from 0.95 to 1.05.

Another embodiment comprises cyclosporin A, Lumulse GMT K®, and an oil having a specific gravity from 0.95 to 1.05.

Another embodiment comprises cyclosporin A, Magrabar GMC®, and an oil having a specific gravity from 0.95 to 1.05.

Another embodiment comprises cyclosporin A, Magrabar GMO-CK®, and an oil having a specific gravity from 0.95 to 1.05.

Another embodiment comprises cyclosporin A, Magrabar MDG-5050®, and an oil having a specific gravity from 0.95 to 1.05.

Another embodiment comprises cyclosporin A, Magrabar PGO-315®, and an oil having a specific gravity from 0.95 to 1.05.

Another embodiment comprises cyclosporin A, Magrabar PGO-1010®, and an oil having a specific gravity from 0.95 to 1.05.

Another embodiment comprises cyclosporin A, Mazol 300K®, and an oil having a specific gravity from 0.95 to 1.05.

Another embodiment comprises cyclosporin A, Mazol GMO-K®, and an oil having a specific gravity from 0.95 to 1.05.

Another embodiment comprises cyclosporin A, Mazol GMS-K®, and an oil having a specific gravity from 0.95 to 1.05.

Another embodiment comprises cyclosporin A, Mazol PG031 -K®, and an oil having a specific gravity from 0.95 to 1.05.

Another embodiment comprises cyclosporin A, Miglyol 812®, and an oil having a specific gravity from 0.95 to 1.05.

Another embodiment comprises cyclosporin A, Norfox 165C®, and an oil having a specific gravity from 0.95 to 1.05.

Another embodiment comprises cyclosporin A, Schercemol GMIS®, and an oil having a specific gravity from 0.95 to 1.05.

Another embodiment comprises cyclosporin A, Tegin®, and an oil having a specific gravity from 0.95 to 1.05.

Another embodiment comprises cyclosporin A, Tegin 4100 Pellets®, and an oil having a specific gravity from 0.95 to 1.05.

Another embodiment comprises cyclosporin A, Tegin M Pellets®, and an oil having a specific gravity from 0.95 to 1.05.

Another embodiment comprises cyclosporin A, Tegin OV®, and an oil having a specific gravity from 0.95 to 1.05.

Another embodiment comprises cyclosporin A, Teginacid H®, and an oil having a specific gravity from 0.95 to 1.05.

Another embodiment comprises cyclosporin A, Tego Cosmo P813®, and an oil having a specific gravity from 0.95 to 1.05.

Another embodiment comprises cyclosporin A, Wickenol 535®, and an oil having a specific gravity from 0.95 to 1.05.

Another embodiment comprises cyclosporin A, Witconol 14®, and an oil having a specific gravity from 0.95 to 1.05.

Another embodiment comprises cyclosporin A, Witconol 14®, and an oil having a specific gravity from 0.95 to 1.05.

Another embodiment comprises cyclosporin A, Witconol 14F®, and an oil having a specific gravity from 0.95 to 1.05.

Another embodiment comprises cyclosporin A, Witconol 18L®, and an oil having a specific gravity from 0.95 to 1.05.

Another embodiment comprises cyclosporin A, Witconol GOT®, and an oil having a specific gravity from 0.95 to 1.05.

Another embodiment comprises cyclosporin A, Witconol MST®, and an oil having a specific gravity from 0.95 to 1.05.

Another embodiment comprises cyclosporin A, Witconol RHT®, and an oil having a specific gravity from 0.95 to 1.05.

Another embodiment comprises cyclosporin A, a Glycol Ester, and an oil having a specific gravity from 0.95 to 1.05.

Another embodiment comprises cyclosporin A, Alkamuls 600 DO®, and an oil having a specific gravity from 0.95 to 1.05.

Another embodiment comprises cyclosporin A, Alkamuls SEG®, and an oil having a specific gravity from 0.95 to 1.05.

Another embodiment comprises cyclosporin A, Atlas EM-2®, and an oil having a specific gravity from 0.95 to 1.05.

Another embodiment comprises cyclosporin A, Cerasynt IP®, and an oil having a specific gravity from 0.95 to 1.05.

Another embodiment comprises cyclosporin A, Cerasynt M®, and an oil having a specific gravity from 0.95 to 1.05.

Another embodiment comprises cyclosporin A, Cerasynt MN®, and an oil having a specific gravity from 0.95 to 1.05.

Another embodiment comprises cyclosporin A, Cerasynt PA®, and an oil having a specific gravity from 0.95 to 1.05.

Another embodiment comprises cyclosporin A, Chemsperse EGDS®, and an oil having a specific gravity from 0.95 to 1.05.

Another embodiment comprises cyclosporin A, Chemsperse EGMS®, and an oil having a specific gravity from 0.95 to 1.05.

Another embodiment comprises cyclosporin A, Colonial Monolaurin®, and an oil having a specific gravity from 0.95 to 1.05.

Another embodiment comprises cyclosporin A, DeMuls SGE-95®, and an oil having a specific gravity from 0.95 to 1.05.

Another embodiment comprises cyclosporin A, Eccoterge 200®, and an oil having a specific gravity from 0.95 to 1.05.

Another embodiment comprises cyclosporin A, Emerest 2380®, and an oil having a specific gravity from 0.95 to 1.05.

Another embodiment comprises cyclosporin A, Ethox 2610®, and an oil having a specific gravity from 0.95 to 1.05.

Another embodiment comprises cyclosporin A, Ethox DO-9®, and an oil having a specific gravity from 0.95 to 1.05.

Another embodiment comprises cyclosporin A, Ethox DO-14®, and an oil having a specific gravity from 0.95 to 1.05.

Another embodiment comprises cyclosporin A, Ethox SO-9®, and an oil having a specific gravity from 0.95 to 1.05.

Another embodiment comprises cyclosporin A, Fizul MD-318®, and an oil having a specific gravity from 0.95 to 1.05.

Another embodiment comprises cyclosporin A, Genapol EGDS-VHP®, and an oil having a specific gravity from 0.95 to 1.05.

Another embodiment comprises cyclosporin A, Genapol TS Powder®, and an oil having a specific gravity from 0.95 to 1.05.

Another embodiment comprises cyclosporin A, Hostacerin WO®, and an oil having a specific gravity from 0.95 to 1.05.

Another embodiment comprises cyclosporin A, Inversol 140®, and an oil having a specific gravity from 0.95 to 1.05.

Another embodiment comprises cyclosporin A, Kemester 104®, and an oil having a specific gravity from 0.95 to 1.05.

Another embodiment comprises cyclosporin A, Kemester 205®, and an oil having a specific gravity from 0.95 to 1.05.

Another embodiment comprises cyclosporin A, Kemester 226®, and an oil having a specific gravity from 0.95 to 1.05.

Another embodiment comprises cyclosporin A, Kemester 5221 SE®, and an oil having a specific gravity from 0.95 to 1.05.

Another embodiment comprises cyclosporin A, Kemester EGDS®, and an oil having a specific gravity from 0.95 to 1.05.

Another embodiment comprises cyclosporin A, Lexemul EGDS®, and an oil having a specific gravity from 0.95 to 1.05.

Another embodiment comprises cyclosporin A, Lexemul EGMS®, and an oil having a specific gravity from 0.95 to 1.05.

Another embodiment comprises cyclosporin A, Lexemul P®, and an oil having a specific gravity from 0.95 to 1.05.

Another embodiment comprises cyclosporin A, Lipo DGLS®, and an oil having a specific gravity from 0.95 to 1.05.

Another embodiment comprises cyclosporin A, Lipo EGDS®, and an oil having a specific gravity from 0.95 to 1.05.

Another embodiment comprises cyclosporin A, Lipo PGMS®, and an oil having a specific gravity from 0.95 to 1.05.

Another embodiment comprises cyclosporin A, Liposorb S-4®, and an oil having a specific gravity from 0.95 to 1.05.

Another embodiment comprises cyclosporin A, Liposorb TO-20®, and an oil having a specific gravity from 0.95 to 1.05.

Another embodiment comprises cyclosporin A, Lumulse PGO®, and an oil having a specific gravity from 0.95 to 1.05.

Another embodiment comprises cyclosporin A, Mackester EGDS®, and an oil having a specific gravity from 0.95 to 1.05.

Another embodiment comprises cyclosporin A, Mackester EGMS®, and an oil having a specific gravity from 0.95 to 1.05.

Another embodiment comprises cyclosporin A, Mackester GSTP®, and an oil having a specific gravity from 0.95 to 1.05.

Another embodiment comprises cyclosporin A, Mackester Series®, and an oil having a specific gravity from 0.95 to 1.05.

Another embodiment comprises cyclosporin A, Magrabar PDG-50®, and an oil having a specific gravity from 0.95 to 1.05.

Another embodiment comprises cyclosporin A, Mapeg 6000 DS®, and an oil having a specific gravity from 0.95 to 1.05.

Another embodiment comprises cyclosporin A, Marlowet 4702®, and an oil having a specific gravity from 0.95 to 1.05.

Another embodiment comprises cyclosporin A, Monalube 305®, and an oil having a specific gravity from 0.95 to 1.05.

Another embodiment comprises cyclosporin A, Monalube 310®, and an oil having a specific gravity from 0.95 to 1.05.

Another embodiment comprises cyclosporin A, Monalube 315®, and an oil having a specific gravity from 0.95 to 1.05.

Another embodiment comprises cyclosporin A, Monalube 320®, and an oil having a specific gravity from 0.95 to 1.05.

Another embodiment comprises cyclosporin A, Monalube 325®, and an oil having a specific gravity from 0.95 to 1.05.

Another embodiment comprises cyclosporin A, Monalube 330®, and an oil having a specific gravity from 0.95 to 1.05.

Another embodiment comprises cyclosporin A, Naturechem PGHS®, and an oil having a specific gravity from 0.95 to 1.05.

Another embodiment comprises cyclosporin A, Polycastorol PLO-840®, and an oil having a specific gravity from 0.95 to 1.05.

Another embodiment comprises cyclosporin A, Polytex 10M®, and an oil having a specific gravity from 0.95 to 1.05.

Another embodiment comprises cyclosporin A, Ritasynt IP®, and an oil having a specific gravity from 0.95 to 1.05.

Another embodiment comprises cyclosporin A, Ross Chem PEG 600 DT®, and an oil having a specific gravity from 0.95 to 1.05.

Another embodiment comprises cyclosporin A, Schercemol PGMS®, and an oil having a specific gravity from 0.95 to 1.05.

Another embodiment comprises cyclosporin A, Sponto H-44C®, and an oil having a specific gravity from 0.95 to 1.05.

Another embodiment comprises cyclosporin A, Tegin G®, and an oil having a specific gravity from 0.95 to 1.05.

Another embodiment comprises cyclosporin A, Witbreak DGE-182®, and an oil having a specific gravity from 0.95 to 1.05.

Another embodiment comprises cyclosporin A, Witbreak DGE-182®, and an oil having a specific gravity from 0.95 to 1.05.

Another embodiment comprises cyclosporin A, Witbreak DRA-21®, and an oil having a specific gravity from 0.95 to 1.05.

Another embodiment comprises cyclosporin A, Witbreak DRA-21®, and an oil having a specific gravity from 0.95 to 1.05.

Another embodiment comprises cyclosporin A, Witbreak DRA-50®, and an oil having a specific gravity from 0.95 to 1.05.

Another embodiment comprises cyclosporin A, Witbreak DRA-50®, and an oil having a specific gravity from 0.95 to 1.05.

Another embodiment comprises cyclosporin A, Witconol F26-46®, and an oil having a specific gravity from 0.95 to 1.05.

Another embodiment comprises cyclosporin A, Witconol H-32®, and an oil having a specific gravity from 0.95 to 1.05.

Another embodiment comprises cyclosporin A, Witconol H-33®, and an oil having a specific gravity from 0.95 to 1.05.

Another embodiment comprises cyclosporin A, Witconol H-35A®, and an oil having a specific gravity from 0.95 to 1.05.

Another embodiment comprises cyclosporin A, Witconol RHP®, and an oil having a specific gravity from 0.95 to 1.05.

Another embodiment comprises cyclosporin A, a Lanolin-based Derivative, and an oil having a specific gravity from 0.95 to 1.05.

Another embodiment comprises cyclosporin A, Amerchol CAB®, and an oil having a specific gravity from 0.95 to 1.05.

Another embodiment comprises cyclosporin A, Amerchol L-101®, and an oil having a specific gravity from 0.95 to 1.05.

Another embodiment comprises cyclosporin A, Amerlate LFA-LO®, and an oil having a specific gravity from 0.95 to 1.05.

Another embodiment comprises cyclosporin A, Amerlate P®, and an oil having a specific gravity from 0.95 to 1.05.

Another embodiment comprises cyclosporin A, Barre Common Degras®, and an oil having a specific gravity from 0.95 to 1.05.

Another embodiment comprises cyclosporin A, Cholesterol NF®, and an oil having a specific gravity from 0.95 to 1.05.

Another embodiment comprises cyclosporin A, Crodalan AWS®, and an oil having a specific gravity from 0.95 to 1.05.

Another embodiment comprises cyclosporin A, Crodalan LA®, and an oil having a specific gravity from 0.95 to 1.05.

Another embodiment comprises cyclosporin A, Emery 1650®, and an oil having a specific gravity from 0.95 to 1.05.

Another embodiment comprises cyclosporin A, Emery 1650®, and an oil having a specific gravity from 0.95 to 1.05.

Another embodiment comprises cyclosporin A, Emery 1740®, and an oil having a specific gravity from 0.95 to 1.05.

Another embodiment comprises cyclosporin A, Emery 1740®, and an oil having a specific gravity from 0.95 to 1.05.

Another embodiment comprises cyclosporin A, Forlan 500®, and an oil having a specific gravity from 0.95 to 1.05.

Another embodiment comprises cyclosporin A, Forlan L®, and an oil having a specific gravity from 0.95 to 1.05.

Another embodiment comprises cyclosporin A, Laneto 50®, and an oil having a specific gravity from 0.95 to 1.05.

Another embodiment comprises cyclosporin A, Laneto 100®, and an oil having a specific gravity from 0.95 to 1.05.

Another embodiment comprises cyclosporin A, Laneto AWS®, and an oil having a specific gravity from 0.95 to 1.05.

Another embodiment comprises cyclosporin A, Lanfrax 1776®, and an oil having a specific gravity from 0.95 to 1.05.

Another embodiment comprises cyclosporin A, Lanfrax 1776®, and an oil having a specific gravity from 0.95 to 1.05.

Another embodiment comprises cyclosporin A, Lanogel 21®, and an oil having a specific gravity from 0.95 to 1.05.

Another embodiment comprises cyclosporin A, Lipolan®, and an oil having a specific gravity from 0.95 to 1.05.

Another embodiment comprises cyclosporin A, Lipolan 31®, and an oil having a specific gravity from 0.95 to 1.05.

Another embodiment comprises cyclosporin A, OHlan®, and an oil having a specific gravity from 0.95 to 1.05.

Another embodiment comprises cyclosporin A, Polychol 5®, and an oil having a specific gravity from 0.95 to 1.05.

Another embodiment comprises cyclosporin A, Polychol 15®, and an oil having a specific gravity from 0.95 to 1.05.

Another embodiment comprises cyclosporin A, Ritacetyl®, and an oil having a specific gravity from 0.95 to 1.05.

Another embodiment comprises cyclosporin A, Ritachol®, and an oil having a specific gravity from 0.95 to 1.05.

Another embodiment comprises cyclosporin A, Ritahydrox®, and an oil having a specific gravity from 0.95 to 1.05.

Another embodiment comprises cyclosporin A, Ritalafa®, and an oil having a specific gravity from 0.95 to 1.05.

Another embodiment comprises cyclosporin A, Ritalan®, and an oil having a specific gravity from 0.95 to 1.05.

Another embodiment comprises cyclosporin A, Ritalan AWS®, and an oil having a specific gravity from 0.95 to 1.05.

Another embodiment comprises cyclosporin A, Ritalan C®, and an oil having a specific gravity from 0.95 to 1.05.

Another embodiment comprises cyclosporin A, Ritawax®, and an oil having a specific gravity from 0.95 to 1.05.

Another embodiment comprises cyclosporin A, Ritawax AEO®, and an oil having a specific gravity from 0.95 to 1.05.

Another embodiment comprises cyclosporin A, Ritawax ALA®, and an oil-having a specific gravity from 0.95 to 1.05.

Another embodiment comprises cyclosporin A, Solan/Solan 50/Super Solan®, and an oil having a specific gravity from 0.95 to 1.05.

Another embodiment comprises cyclosporin A, Super Hartolan/Hartolan®, and an oil having a specific gravity from 0.95 to 1.05.

Another embodiment comprises cyclosporin A, Supersat AWS-4®, and an oil having a specific gravity from 0.95 to 1.05.

Another embodiment comprises cyclosporin A, Supersat AWS-24®, and an oil having a specific gravity from 0.95 to 1.05.

Another embodiment comprises cyclosporin A, a Lecithin or Lecithin Derivative, and an oil having a specific gravity from 0.95 to 1.05.

Another embodiment comprises cyclosporin A, Alcolec®, and an oil having a specific gravity from 0.95 to 1.05.

Another embodiment comprises cyclosporin A, Lecithin®, and an oil having a specific gravity from 0.95 to 1.05.

Another embodiment comprises cyclosporin A, Lexin K®, and an oil having a specific gravity from 0.95 to 1.05.

Another embodiment comprises cyclosporin A, Natipide®, and an oil having a specific gravity from 0.95 to 1.05.

Another embodiment comprises cyclosporin A, a Lignin or Lignin Derivative, and an oil having a specific gravity from 0.95 to 1.05.

Another embodiment comprises cyclosporin A, Diwatex XP 9®, and an oil having a specific gravity from 0.95 to 1.05.

Another embodiment comprises cyclosporin A, Dynasperse LCD®, and an oil having a specific gravity from 0.95 to 1.05.

Another embodiment comprises cyclosporin A, Indulin SAL®, and an oil having a specific gravity from 0.95 to 1.05.

Another embodiment comprises cyclosporin A, Indulin W-1®, and an oil having a specific gravity from 0.95 to 1.05.

Another embodiment comprises cyclosporin A, Indulin W-5®, and an oil having a specific gravity from 0.95 to 1.05.

Another embodiment comprises cyclosporin A, Lignosol FTA®, and an oil having a specific gravity from 0.95 to 1.05.

Another embodiment comprises cyclosporin A, Lignosol SFX-65®, and an oil having a specific gravity from 0.95 to 1.05.

Another embodiment comprises cyclosporin A, Marasperse 52 CP®, and an oil having a specific gravity from 0.95 to 1.05.

Another embodiment comprises cyclosporin A, Marasperse AG®, and an oil having a specific gravity from 0.95 to 1.05.

Another embodiment comprises cyclosporin A, Marasperse CBOS-4®, and an oil having a specific gravity from 0.95 to 1.05.

Another embodiment comprises cyclosporin A, Ufoxane 2®, and an oil having a specific gravity from 0.95 to 1.05.

Another embodiment comprises cyclosporin A, a Methyl Ester, and an oil having a specific gravity from 0.95 to 1.05.

Another embodiment comprises cyclosporin A, E.B. Cleaner AK®, and an oil having a specific gravity from 0.95 to 1.05.

Another embodiment comprises cyclosporin A, Oleocal ME-70®, and an oil having a specific gravity from 0.95 to 1.05.

Another embodiment comprises cyclosporin A, Trade Name Company®, and an oil having a specific gravity from 0.95 to 1.05.

Another embodiment comprises cyclosporin A, Methyl Esters (cont'd)®, and an oil having a specific gravity from 0.95 to 1.05.

Another embodiment comprises cyclosporin A, Oleocal ME-92®, and an oil having a specific gravity from 0.95 to 1.05.

Another embodiment comprises cyclosporin A, Oleocal ME-112®, and an oil having a specific gravity from 0.95 to 1.05.

Another embodiment comprises cyclosporin A, Oleocal ME-130®, and an oil having a specific gravity from 0.95 to 1.05.

Another embodiment comprises cyclosporin A, a Monoglyceride or a Derivative thereof, and an oil having a specific gravity from 0.95 to 1.05.

Another embodiment comprises cyclosporin A, Dynacet 211®, and an oil having a specific gravity from 0.95 to 1.05.

Another embodiment comprises cyclosporin A, Hetsorb S-20®, and an oil having a specific gravity from 0.95 to 1.05.

Another embodiment comprises cyclosporin A, Imwitor 191®, and an oil having a specific gravity from 0.95 to 1.05.

Another embodiment comprises cyclosporin A, Imwitor 370®, and an oil having a specific gravity from 0.95 to 1.05.

Another embodiment comprises cyclosporin A, Imwitor 375®, and an oil having a specific gravity from 0.95 to 1.05.

Another embodiment comprises cyclosporin A, Imwitor 900®, and an oil having a specific gravity from 0.95 to 1.05.

Another embodiment comprises cyclosporin A, Imwitor 945®, and an oil having a specific gravity from 0.95 to 1.05.

Another embodiment comprises cyclosporin A, Imwitor 2020®, and an oil having a specific gravity from 0.95 to 1.05.

Another embodiment comprises cyclosporin A, Kemester 5500®, and an oil having a specific gravity from 0.95 to 1.05.

Another embodiment comprises cyclosporin A, Kemester 6000®, and an oil having a specific gravity from 0.95 to 1.05.

Another embodiment comprises cyclosporin A, Magrabar GMC®, and an oil having a specific gravity from 0.95 to 1.05.

Another embodiment comprises cyclosporin A, Magrabar GMO-CK®, and an oil having a specific gravity from 0.95 to 1.05.

Another embodiment comprises cyclosporin A, Magrabar GPC-10®, and an oil having a specific gravity from 0.95 to 1.05.

Another embodiment comprises cyclosporin A, Magrabar MDG-5050®, and an oil having a specific gravity from 0.95 to 1.05.

Another embodiment comprises cyclosporin A, Monalube 335®, and an oil having a specific gravity from 0.95 to 1.05.

Another embodiment comprises cyclosporin A, Monoglycerides Glyceryl Monestearate Archer Daniels Midland Company®, and an oil having a specific gravity from 0.95 to 1.05.

Another embodiment comprises cyclosporin A, Rita GMS®, and an oil having a specific gravity from 0.95 to 1.05.

Another embodiment comprises cyclosporin A, Ritamulse SCG®, and an oil having a specific gravity from 0.95 to 1.05.

Another embodiment comprises cyclosporin A, Softigen 701®, and an oil having a specific gravity from 0.95 to 1.05.

Another embodiment comprises cyclosporin A, Tally 100 Plus®, and an oil having a specific gravity from 0.95 to 1.05.

Another embodiment comprises cyclosporin A, a Polyethylene Glycol, and an oil having a specific gravity from 0.95 to 1.05.

Another embodiment comprises cyclosporin A, Emulgade PL 68/50®, and an oil having a specific gravity from 0.95 to 1.05.

Another embodiment comprises cyclosporin A, Lumulse PEG®, and an oil having a specific gravity from 0.95 to 1.05.

Another embodiment comprises cyclosporih A, Rhodasurf PEG-400®, and an oil having a specific gravity from 0.95 to 1.05.

Another embodiment comprises cyclosporin A, Rhodasurf PEG-600®, and an oil having a specific gravity from 0.95 to 1.05.

Another embodiment comprises cyclosporin A, Witconol PEG-400®, and an oil having a specific gravity from 0.95 to 1.05.

Another embodiment comprises cyclosporin A, a Polymeric Surfactant, and an oil having a specific gravity from 0.95 to 1.05.

Another embodiment comprises cyclosporin A, Acritamer PNC-EG®, and an oil having a specific gravity from 0.95 to 1.05.

Another embodiment comprises cyclosporin A, Ag-Rho DEP-775®, and an oil having a specific gravity from 0.95 to 1.05.

Another embodiment comprises cyclosporin A, APG 325N Glycoside®, and an oil having a specific gravity from 0.95 to 1.05.

Another embodiment comprises cyclosporin A, Aristoflex AVC®, and an oil having a specific gravity from 0.95 to 1.05.

Another embodiment comprises cyclosporin A, Aristoflex HMB®, and an oil having a specific gravity from 0.95 to 1.05.

Another embodiment comprises cyclosporin A, Burco NPS-225®, and an oil having a specific gravity from 0.95 to 1.05.

Another embodiment comprises cyclosporin A, Burco NPS-816®, and an oil having a specific gravity from 0.95 to 1.05.

Another embodiment comprises cyclosporin A, Chemccinate 5603®, and an oil having a specific gravity from 0.95 to 1.05.

Another embodiment comprises cyclosporin A, Cosmedia Guar C-261 N®, and an oil having a specific gravity from 0.95 to 1.05.

Another embodiment comprises cyclosporin A, Gantrez S-95®, and an oil having a specific gravity from 0.95 to 1.05.

Another embodiment comprises cyclosporin A, Glucopon 220 UP®, and an oil having a specific gravity from 0.95 to 1.05.

Another embodiment comprises cyclosporin A, Glucopon 225 DK®, and an oil having a specific gravity from 0.95 to 1.05.

Another embodiment comprises cyclosporin A, Glucopon 425 N®, and an oil having a specific gravity from 0.95 to 1.05.

Another embodiment comprises cyclosporin A, Glucopon 600 UP®, and an oil having a specific gravity from 0.95 to 1.05.

Another embodiment comprises cyclosporin A, Glucopon 625 UP®, and an oil having a specific gravity from 0.95 to 1.05.

Another embodiment comprises cyclosporin A, Pemulen 1621®, and an oil having a specific gravity from 0.95 to 1.05.

Another embodiment comprises cyclosporin A, Pemulen 1622®, and an oil having a specific gravity from 0.95 to 1.05.

Another embodiment comprises cyclosporin A, Pemulen TR-1®, and an oil having a specific gravity from 0.95 to 1.05.

Another embodiment comprises cyclosporin A, Pemulen TR-2®, and an oil having a specific gravity from 0.95 to 1.05.

Another embodiment comprises cyclosporin A, Plantacare 818®, and an oil having a specific gravity from 0.95 to 1.05.

Another embodiment comprises cyclosporin A, Plantapon LGC Sorb®, and an oil having a specific gravity from 0.95 to 1.05.

Another embodiment comprises cyclosporin A, Plantaren 1200N®, and an oil having a specific gravity from 0.95 to 1.05.

Another embodiment comprises cyclosporin A, Plantaren 2000N®, and an oil having a specific gravity from 0.95 to 1.05.

Another embodiment comprises cyclosporin A, Viscolam AT 64®, and an oil having a specific gravity from 0.95 to 1.05.

Another embodiment comprises cyclosporin A, Viscolam AT 64P®, and an oil having a specific gravity from 0.95 to 1.05.

Another embodiment comprises cyclosporin A, Viscolam AT 100®, and an oil having a specific gravity from 0.95 to 1.05.

Another embodiment comprises cyclosporin A, Viscolam MAC 7®, and an oil having a specific gravity from 0.95 to 1.05.

Another embodiment comprises cyclosporin A, Viscolam SMC 20®, and an oil having a specific gravity from 0.95 to 1.05.

Another embodiment comprises cyclosporin A, Witbreak RTC-323®, and an oil having a specific gravity from 0.95 to 1.05.

Another embodiment comprises cyclosporin A, WSI 3700®, and an oil having a specific gravity from 0.95 to 1.05.

Another embodiment comprises cyclosporin A, a Propoxylated or Ethoxylated Fatty Acid, Alcohol, or Alkyl Phenol, and an oil having a specific gravity from 0.95 to 1.05.

Another embodiment comprises cyclosporin A, Antarox AA-60®, and an oil having a specific gravity from 0.95 to 1.05.

Another embodiment comprises cyclosporin A, Antarox LF-224®, and an oil having a specific gravity from 0.95 to 1.05.

Another embodiment comprises cyclosporin A, Burcomul DFE-45®, and an oil having a specific gravity from 0.95 to 1.05.

Another embodiment comprises cyclosporin A, Burcoterge LFE-1000®, and an oil having a specific gravity from 0.95 to 1.05.

Another embodiment comprises cyclosporin A, Chemal LF-25B®, and an oil having a specific gravity from 0.95 to 1.05.

Another embodiment comprises cyclosporin A, Chemal LF-40B®, and an oil having a specific gravity from 0.95 to 1.05.

Another embodiment comprises cyclosporin A, Dehypon LS-36®, and an oil having a specific gravity from 0.95 to 1.05.

Another embodiment comprises cyclosporin A, Dehypon LS-36®, and an oil having a specific gravity from 0.95 to 1.05.

Another embodiment comprises cyclosporin A, Dehypon LS-54®, and an oil having a specific gravity from 0.95 to 1.05.

Another embodiment comprises cyclosporin A, Dehypon LS-54®, and an oil having a specific gravity from 0.95 to 1.05.

Another embodiment comprises cyclosporin A, Delonic 100 VLF®, and an oil having a specific gravity from 0.95 to 1.05.

Another embodiment comprises cyclosporin A, Delonic LF-60 MOD®, and an oil having a specific gravity from 0.95 to 1.05.

Another embodiment comprises cyclosporin A, Empiderm B®, and an oil having a specific gravity from 0.95 to 1.05.

Another embodiment comprises cyclosporin A, Ethylan 1206®, and an oil having a specific gravity from 0.95 to 1.05.

Another embodiment comprises cyclosporin A, Ethylan NS-500K®, and an oil having a specific gravity from 0.95 to 1.05.

Another embodiment comprises cyclosporin A, Ethylan NS-500LQ®, and an oil having a specific gravity from 0.95 to 1.05.

Another embodiment comprises cyclosporin A, Genapol 1392®, and an oil having a specific gravity from 0.95 to 1.05.

Another embodiment comprises cyclosporin A, Genapol 2317®, and an oil having a specific gravity from 0.95 to 1.05.

Another embodiment comprises cyclosporin A, Genapol 26EP710®, and an oil having a specific gravity from 0.95 to 1.05.

Another embodiment comprises cyclosporin A, Genapol EP 1022®, and an oil having a specific gravity from 0.95 to 1.05.

Another embodiment comprises cyclosporin A, Genapol EP 1024®, and an oil having a specific gravity from 0.95 to 1.05.

Another embodiment comprises cyclosporin A, Genapol EP 6068®, and an oil having a specific gravity from 0.95 to 1.05.

Another embodiment comprises cyclosporin A, Genapol NP915®, and an oil having a specific gravity from 0.95 to 1.05.

Another embodiment comprises cyclosporin A, Kieralon MFB®, and an oil having a specific gravity from 0.95 to 1.05.

Another embodiment comprises cyclosporin A, Lumisolve CSA-80 V®, and an oil having a specific gravity from 0.95 to 1.05.

Another embodiment comprises cyclosporin A, Marlowet 5001®, and an oil having a specific gravity from 0.95 to 1.05.

Another embodiment comprises cyclosporin A, Marlox FK 64®, and an oil having a specific gravity from 0.95 to 1.05.

Another embodiment comprises cyclosporin A, Marlox MO 124®, and an oil having a specific gravity from 0.95 to 1.05.

Another embodiment comprises cyclosporin A, Marlox S 58®, and an oil having a specific gravity from 0.95 to 1.05.

Another embodiment comprises cyclosporin A, Nonatell 1003®, and an oil having a specific gravity from 0.95 to 1.05.

Another embodiment comprises cyclosporin A, Nonatell 1038®, and an oil having a specific gravity from 0.95 to 1.05.

Another embodiment comprises cyclosporin A, Nonatell 1052®, and an oil having a specific gravity from 0.95 to 1.05.

Another embodiment comprises cyclosporin A, Nonatell 1061®, and an oil having a specific gravity from 0.95 to 1.05.

Another embodiment comprises cyclosporin A, Nonatell 1075®, and an oil having a specific gravity from 0.95 to 1.05.

Another embodiment comprises cyclosporin A, Nonatell 1088®, and an oil having a specific gravity from 0.95 to 1.05.

Another embodiment comprises cyclosporin A, Nonatell 1123®, and an oil having a specific gravity from 0.95 to 1.05.

Another embodiment comprises cyclosporin A, Nonatell 1153®, and an oil having a specific gravity from 0.95 to 1.05.

Another embodiment comprises cyclosporin A, Nonatell 1161®, and an oil having a specific gravity from 0.95 to 1.05.

Another embodiment comprises cyclosporin A, Nonatell 1172®, and an oil having a specific gravity from 0.95 to 1.05.

Another embodiment comprises cyclosporin A, Nonatell 1181®, and an oil having a specific gravity from 0.95 to 1.05.

Another embodiment comprises cyclosporin A, Norfox 36®, and an oil having a specific gravity from 0.95 to 1.05.

Another embodiment comprises cyclosporin A, Procetyl AWS®, and an oil having a specific gravity from 0.95 to 1.05.

Another embodiment comprises cyclosporin A, Sandoxylate SX 412®, and an oil having a specific gravity from 0.95 to 1.05.

Another embodiment comprises cyclosporin A, Sandoxylate SX 418®, and an oil having a specific gravity from 0.95 to 1.05.

Another embodiment comprises cyclosporin A, Surfonic JL-80X®, and an oil having a specific gravity from 0.95 to 1.05.

Another embodiment comprises cyclosporin A, Surfonic JL-80X-B1®, and an oil having a specific gravity from 0.95 to 1.05.

Another embodiment comprises cyclosporin A, Surfonic L4-29X®, and an oil having a specific gravity from 0.95 to 1.05.

Another embodiment comprises cyclosporin A, Surfonic LF®, and an oil having a specific gravity from 0.95 to 1.05.

Another embodiment comprises cyclosporin A, T-Det A826®, and an oil having a specific gravity from 0.95 to 1.05.

Another embodiment comprises cyclosporin A, T-Det LF-416®, and an oil having a specific gravity from 0.95 to 1.05.

Another embodiment comprises cyclosporin A, Tergitol Min-Foam 1X®, and an oil having a specific gravity from 0.95 to 1.05.

Another embodiment comprises cyclosporin A, Tergitol Min-Foam 2X®, and an oil having a specific gravity from 0.95 to 1.05.

Another embodiment comprises cyclosporin A, Triton CF-21®, and an oil having a specific gravity from 0.95 to 1.05.

Another embodiment comprises cyclosporin A, Triton CF-76®, and an oil having a specific gravity from 0.95 to 1.05.

Another embodiment comprises cyclosporin A, Triton XL-80N®, and an oil having a specific gravity from 0.95 to 1.05.

Another embodiment comprises cyclosporin A, Witconol NS-98®, and an oil having a specific gravity from 0.95 to 1.05.

Another embodiment comprises cyclosporin A, Witconol NS-108LQ®, and an oil having a specific gravity from 0.95 to 1.05.

Another embodiment comprises cyclosporin A, Witconol NS-145®, and an oil having a specific gravity from 0.95 to 1.05.

Another embodiment comprises cyclosporin A, Witconol NS-179®, and an oil having a specific gravity from 0.95 to 1.05.

Another embodiment comprises cyclosporin A, a Protein-based Surfactant, and an oil having a specific gravity from 0.95 to 1.05.

Another embodiment comprises cyclosporin A, AminoFoam W®, and an oil having a specific gravity from 0.95 to 1.05.

Another embodiment comprises cyclosporin A, Amiter LGOD-2®, and an oil having a specific gravity from 0.95 to 1.05.

Another embodiment comprises cyclosporin A, Amiter LGS-2®, and an oil having a specific gravity from 0.95 to 1.05.

Another embodiment comprises cyclosporin A, Amiter LGS-5®, and an oil having a specific gravity from 0.95 to 1.05.

Another embodiment comprises cyclosporin A, Lamepon S®, and an oil having a specific gravity from 0.95 to 1.05.

Another embodiment comprises cyclosporin A, Lamepon S®, and an oil having a specific gravity from 0.95 to 1.05.

Another embodiment comprises cyclosporin A, Maypon 4C®, and an oil having a specific gravity from 0.95 to 1.05.

Another embodiment comprises cyclosporin A, May-Tein C®, and an oil having a specific gravity from 0.95 to 1.05.

Another embodiment comprises cyclosporin A, May-Tein CT®, and an oil having a specific gravity from 0.95 to 1.05.

Another embodiment comprises cyclosporin A, May-Tein KTS®, and an oil having a specific gravity from 0.95 to 1.05.

Another embodiment comprises cyclosporin A, May-Tein SY®, and an oil having a specific gravity from 0.95 to 1.05.

Another embodiment comprises cyclosporin A, Plantapon S®, and an oil having a specific gravity from 0.95 to 1.05.

Another embodiment comprises cyclosporin A, Proteol APL®, and an oil having a specific gravity from 0.95 to 1.05.

Another embodiment comprises cyclosporin A, Proteol OAT®, and an oil having a specific gravity from 0.95 to 1.05.

Another embodiment comprises cyclosporin A, Pyroter CPI-40®, and an oil having a specific gravity from 0.95 to 1.05.

Another embodiment comprises cyclosporin A, Pyroter GPI-25®, and an oil having a specific gravity from 0.95 to 1.05.

Another embodiment comprises cyclosporin A, Supro-Tein S®, and an oil having a specific gravity from 0.95 to 1.05.

Another embodiment comprises cyclosporin A, Supro-Tein V®, and an oil having a specific gravity from 0.95 to 1.05.

Another embodiment comprises cyclosporin A, a Sarcosine Derivative®, and an oil having a specific gravity from 0.95 to 1.05.

Another embodiment comprises cyclosporin A, Crodasinic LS-30®, and an oil having a specific gravity from 0.95 to 1.05.

Another embodiment comprises cyclosporin A, Vanseal CS®, and an oil having a specific gravity from 0.95 to 1.05.

Another embodiment comprises cyclosporin A, Vanseal LS®, and an oil having a specific gravity from 0.95 to 1.05.

Another embodiment comprises cyclosporin A, Vanseal MS®, and an oil having a specific gravity from 0.95 to 1.05.

Another embodiment comprises cyclosporin A, Vanseal NACS-30®, and an oil having a specific gravity from 0.95 to 1.05.

Another embodiment comprises cyclosporin A, Vanseal NALS-95®, and an oil having a specific gravity from 0.95 to 1.05.

Another embodiment comprises cyclosporin A, Vanseal OS®, and an oil having a specific gravity from 0.95 to 1.05.

Another embodiment comprises cyclosporin A, a Silicone-based Surfactant®, and an oil having a specific gravity from 0.95 to 1.05.

Another embodiment comprises cyclosporin A, Abil-B-9950®, and an oil having a specific gravity from 0.95 to 1.05.

Another embodiment comprises cyclosporin A, Abil Care 85®, and an oil having a specific gravity from 0.95 to 1.05.

Another embodiment comprises cyclosporin A, Abil EM 90®, and an oil having a specific gravity from 0.95 to 1.05.

Another embodiment comprises cyclosporin A, Abil EM 97®, and an oil having a specific gravity from 0.95 to 1.05.

Another embodiment comprises cyclosporin A, Abil WE-09®, and an oil having a specific gravity from 0.95 to 1.05.

Another embodiment comprises cyclosporin A, Dow Corning 1248 Fluid®, and an oil having a specific gravity from 0.95 to 1.05.

Another embodiment comprises cyclosporin A, Dow Corning 3225C®, and an oil having a specific gravity from 0.95 to 1.05.

Another embodiment comprises cyclosporin A, Dow Corning 5200®, and an oil having a specific gravity from 0.95 to 1.05.

Another embodiment comprises cyclosporin A, Dow Corning Q4-3667®, and an oil having a specific gravity from 0.95 to 1.05.

Another embodiment comprises cyclosporin A, Monasil PCA®, and an oil having a specific gravity from 0.95 to 1.05.

Another embodiment comprises cyclosporin A, Monasil PDM®, and an oil having a specific gravity from 0.95 to 1.05.

Another embodiment comprises cyclosporin A, Monasil PLN®, and an oil having a specific gravity from 0.95 to 1.05.

Another embodiment comprises cyclosporin A, Polyderm PPI-SI-WS®, and an oil having a specific gravity from 0.95 to 1.05.

Another embodiment comprises cyclosporin A, Troysol 380W®, and an oil having a specific gravity from 0.95 to 1.05.

Another embodiment comprises cyclosporin A, Troysol S366®, and an oil having a specific gravity from 0.95 to 1.05.

Another embodiment comprises cyclosporin A, a Sorbitan Derivative, and an oil having a specific gravity from 0.95 to 1.05.

Another embodiment comprises cyclosporin A, Alkamuls SML®, and an oil having a specific gravity from 0.95 to 1.05.

Another embodiment comprises cyclosporin A, Alkamuls SMO®, and an oil having a specific gravity from 0.95 to 1.05.

Another embodiment comprises cyclosporin A, Alkamuls STO®, and an oil having a specific gravity from 0.95 to 1.05.

Another embodiment comprises cyclosporin A, Arlacel 20®, and an oil having a specific gravity from 0.95 to 1.05.

Another embodiment comprises cyclosporin A, Arlacel 40®, and an oil having a specific gravity from 0.95 to 1.05.

Another embodiment comprises cyclosporin A, Arlacel 60®, and an oil having a specific gravity from 0.95 to 1.05.

Another embodiment comprises cyclosporin A, Arlacel 80®, and an oil having a specific gravity from 0.95 to 1.05.

Another embodiment comprises cyclosporin A, Arlacel C®, and an oil having a specific gravity from 0.95 to 1.05.

Another embodiment comprises cyclosporin A, Armul 21®, and an oil having a specific gravity from 0.95 to 1.05.

Another embodiment comprises cyclosporin A, Atlox 80®, and an oil having a specific gravity from 0.95 to 1.05.

Another embodiment comprises cyclosporin A, Atlox 847®, and an oil having a specific gravity from 0.95 to 1.05.

Another embodiment comprises cyclosporin A, Atlox 1045A®, and an oil having a specific gravity from 0.95 to 1.05.

Another embodiment comprises cyclosporin A, Canarcel 20®, and an oil having a specific gravity from 0.95 to 1.05.

Another embodiment comprises cyclosporin A, Canarcel 60®, and an oil having a specific gravity from 0.95 to 1.05.

Another embodiment comprises cyclosporin A, Canarcel 80®, and an oil having a specific gravity from 0.95 to 1.05.

Another embodiment comprises cyclosporin A, Canarcel TW 20®, and an oil having a specific gravity from 0.95 to 1.05.

Another embodiment comprises cyclosporin A, Canarcel TW 60®, and an oil having a specific gravity from 0.95 to 1.05.

Another embodiment comprises cyclosporin A, Canarcel TW 80®, and an oil having a specific gravity from 0.95 to 1.05.

Another embodiment comprises cyclosporin A, Coladet BSB-P®, and an oil having a specific gravity from 0.95 to 1.05.

Another embodiment comprises cyclosporin A, Customulse O-20®, and an oil having a specific gravity from 0.95 to 1.05.

Another embodiment comprises cyclosporin A, Dehymuls E®, and an oil having a specific gravity from 0.95 to 1.05.

Another embodiment comprises cyclosporin A, DeSotan SMO®, and an oil having a specific gravity from 0.95 to 1.05.

Another embodiment comprises cyclosporin A, DeSotan SMO-20®, and an oil having a specific gravity from 0.95 to 1.05.

Another embodiment comprises cyclosporin A, DeSotan SMT®, and an oil having a specific gravity from 0.95 to 1.05.

Another embodiment comprises cyclosporin A, DeSotan SMT-20®, and an oil having a specific gravity from 0.95 to 1.05.

Another embodiment comprises cyclosporin A, Durfax 60®, and an oil having a specific gravity from 0.95 to 1.05.

Another embodiment comprises cyclosporin A, Durfax 65®, and an oil having a specific gravity from 0.95 to 1.05.

Another embodiment comprises cyclosporin A, Durfax 80®, and an oil having a specific gravity from 0.95 to 1.05.

Another embodiment comprises cyclosporin A, Durtan 60®, and an oil having a specific gravity from 0.95 to 1.05.

Another embodiment comprises cyclosporin A, Durtan 65®, and an oil having a specific gravity from 0.95 to 1.05.

Another embodiment comprises cyclosporin A, Liposorb L®, and an oil having a specific gravity from 0.95 to 1.05.

Another embodiment comprises cyclosporin A, Liposorb L-10®, and an oil having a specific gravity from 0.95 to 1.05.

Another embodiment comprises cyclosporin A, Liposorb L-20®, and an oil having a specific gravity from 0.95 to 1.05.

Another embodiment comprises cyclosporin A, Liposorb O®, and an oil having a specific gravity from 0.95 to 1.05.

Another embodiment comprises cyclosporin A, Liposorb O-20®, and an oil having a specific gravity from 0.95 to 1.05.

Another embodiment comprises cyclosporin A, Liposorb P®, and an oil having a specific gravity from 0.95 to 1.05.

Another embodiment comprises cyclosporin A, Liposorb P-20®, and an oil having a specific gravity from 0.95 to 1.05.

Another embodiment comprises cyclosporin A, Liposorb S®, and an oil having a specific gravity from 0.95 to 1.05.

Another embodiment comprises cyclosporin A, Liposorb S-20®, and an oil having a specific gravity from 0.95 to 1.05.

Another embodiment comprises cyclosporin A, Liposorb SQO®, and an oil having a specific gravity from 0.95 to 1.05.

Another embodiment comprises cyclosporin A, Liposorb TO®, and an oil having a specific gravity from 0.95 to 1.05.

Another embodiment comprises cyclosporin A, Liposorb TS®, and an oil having a specific gravity from 0.95 to 1.05.

Another embodiment comprises cyclosporin A, Liposorb TS-20®, and an oil having a specific gravity from 0.95 to 1.05.

Another embodiment comprises cyclosporin A, Lumisorb PS®, and an oil having a specific gravity from 0.95 to 1.05.

Another embodiment comprises cyclosporin A, Lumisorb SMO (T)®, and an oil having a specific gravity from 0.95 to 1.05.

Another embodiment comprises cyclosporin A, Lumisorb SMS K®, and an oil having a specific gravity from 0.95 to 1.05.

Another embodiment comprises cyclosporin A, Lumisorb SSO®, and an oil having a specific gravity from 0.95 to 1.05.

Another embodiment comprises cyclosporin A, Lumisorb STS K®, and an oil having a specific gravity from 0.95 to 1.05.

Another embodiment comprises cyclosporin A, Lumisorb STT®, and an oil having a specific gravity from 0.95 to 1.05.

Another embodiment comprises cyclosporin A, Magrabar SMO®, and an oil having a specific gravity from 0.95 to 1.05.

Another embodiment comprises cyclosporin A, Magrabar SMO-VEG®, and an oil having a specific gravity from 0.95 to 1.05.

Another embodiment comprises cyclosporin A, Magrabar SMT®, and an oil having a specific gravity from 0.95 to 1.05.

Another embodiment comprises cyclosporin A, Magrabar STO®, and an oil having a specific gravity from 0.95 to 1.05.

Another embodiment comprises cyclosporin A, Miracare BC-27®, and an oil having a specific gravity from 0.95 to 1.05.

Another embodiment comprises cyclosporin A, Ritabate 20®, and an oil having a specific gravity from 0.95 to 1.05.

Another embodiment comprises cyclosporin A, Ritabate 40®, and an oil having a specific gravity from 0.95 to 1.05.

Another embodiment comprises cyclosporin A, Ritabate 60®, and an oil having a specific gravity from 0.95 to 1.05.

Another embodiment comprises cyclosporin A, Ritabate 80®, and an oil having a specific gravity from 0.95 to 1.05.

Another embodiment comprises cyclosporin A, T-Maz®, and an oil having a specific gravity from 0.95 to 1.05.

Another embodiment comprises cyclosporin A, Tego SML®, and an oil having a specific gravity from 0.95 to 1.05.

Another embodiment comprises cyclosporin A, Tego SML 20®, and an oil having a specific gravity from 0.95 to 1.05.

Another embodiment comprises cyclosporin A, Tego SMO 80 V®, and an oil having a specific gravity from 0.95 to 1.05.

Another embodiment comprises cyclosporin A, Tego SMO V®, and an oil having a specific gravity from 0.95 to 1.05.

Another embodiment comprises cyclosporin A, Tego SMS®, and an oil having a specific gravity from 0.95 to 1.05.

Another embodiment comprises cyclosporin A, Tego STO V®, and an oil having a specific gravity from 0.95 to 1.05.

Another embodiment comprises cyclosporin A, Tween 21®, and an oil having a specific gravity from 0.95 to 1.05.

Another embodiment comprises cyclosporin A, Tween 40®, and an oil having a specific gravity from 0.95 to 1.05.

Another embodiment comprises cyclosporin A, Tween 60®, and an oil having a specific gravity from 0.95 to 1.05.

Another embodiment comprises cyclosporin A, Tween 60 K®, and an oil having a specific gravity from 0.95 to 1.05.

Another embodiment comprises cyclosporin A, Tween 61®, and an oil having a specific gravity from 0.95 to 1.05.

Another embodiment comprises cyclosporin A, Tween 65®, and an oil having a specific gravity from 0.95 to 1.05.

Another embodiment comprises cyclosporin A, Tween 80®, and an oil having a specific gravity from 0.95 to 1.05.

Another embodiment comprises cyclosporin A, Tween 80 K®, and an oil having a specific gravity from 0.95 to 1.05.

Another embodiment comprises cyclosporin A, Tween 81®, and an oil having a specific gravity from 0.95 to 1.05.

Another embodiment comprises cyclosporin A, Tween 85®, and an oil having a specific gravity from 0.95 to 1.05.

Another embodiment comprises cyclosporin A, a Sucrose or Glucose Ester, or Derivative thereof, and an oil having a specific gravity from 0.95 to 1.05.

Another embodiment comprises cyclosporin A, DeSulf GOS-P-60WCG®, and an oil having a specific gravity from 0.95 to 1.05.

Another embodiment comprises cyclosporin A, Glucam E-20 Distearate®, and an oil having a specific gravity from 0.95 to 1.05.

Another embodiment comprises cyclosporin A, Glucamate DOE-120®, and an oil having a specific gravity from 0.95 to 1.05.

Another embodiment comprises cyclosporin A, Glucamate SSE-20®, and an oil having a specific gravity from 0.95 to 1.05.

Another embodiment comprises cyclosporin A, Glucate DO®, and an oil having a specific gravity from 0.95 to 1.05.

Another embodiment comprises cyclosporin A, Glucate SS®, and an oil having a specific gravity from 0.95 to 1.05.

Another embodiment comprises cyclosporin A, Glucopon 425 UP®, and an oil having a specific gravity from 0.95 to 1.05.

Another embodiment comprises cyclosporin A, Isolan IS®, and an oil having a specific gravity from 0.95 to 1.05.

Another embodiment comprises cyclosporin A, Mazon 40®, and an oil having a specific gravity from 0.95 to 1.05.

Another embodiment comprises cyclosporin A, Montanov 82®, and an oil having a specific gravity from 0.95 to 1.05.

Another embodiment comprises cyclosporin A, Montanov 202®, and an oil having a specific gravity from 0.95 to 1.05.

Another embodiment comprises cyclosporin A, Montanov S®, and an oil having a specific gravity from 0.95 to 1.05.

Another embodiment comprises cyclosporin A, Rheozan®, and an oil having a specific gravity from 0.95 to 1.05.

Another embodiment comprises cyclosporin A, Simulsol AS 48®, and an oil having a specific gravity from 0.95 to 1.05.

Another embodiment comprises cyclosporin A, Simulsol SL 4®, and an oil having a specific gravity from 0.95 to 1.05.

Another embodiment comprises cyclosporin A, Simulsol SL 10®, and an oil having a specific gravity from 0.95 to 1.05.

Another embodiment comprises cyclosporin A, Simulsol SL 11W®, and an oil having a specific gravity from 0.95 to 1.05.

Another embodiment comprises cyclosporin A, Simulsol SL 55®, and an oil having a specific gravity from 0.95 to 1.05.

Another embodiment comprises cyclosporin A, Suga Nate 100 and 160®, and an oil having a specific gravity from 0.95 to 1.05.

Another embodiment comprises cyclosporin A, Tego Care 450®, and an oil having a specific gravity from 0.95 to 1.05.

Another embodiment comprises cyclosporin A, Tego Care CG 90®, and an oil having a specific gravity from 0.95 to 1.05.

Another embodiment comprises cyclosporin A, Tego Care PS®, and an oil having a specific gravity from 0.95 to 1.05.

Another embodiment comprises cyclosporin A, Tegosoft PSE 141 G®, and an oil having a specific gravity from 0.95 to 1.05.

Another embodiment comprises cyclosporin A, Tegotens G 826®, and an oil having a specific gravity from 0.95 to 1.05.

Another embodiment comprises cyclosporin A, Triton BG-10 (70%)®, and an oil having a specific gravity from 0.95 to 1.05.

Another embodiment comprises cyclosporin A, Triton CG-110 (60%)®, and an oil having a specific gravity from 0.95 to 1.05.

Another embodiment comprises cyclosporin A, Wickenol 545®, and an oil having a specific gravity from 0.95 to 1.05.

Another embodiment comprises cyclosporin A, an alcohol, and Anise oil.

Another embodiment comprises cyclosporin A, Diglycerol®, and Anise oil.

Another embodiment comprises cyclosporin A, Hetoxide GT-80®, and Anise oil.

Another embodiment comprises cyclosporin A, Lexemul BEO®, and Anise oil.

Another embodiment comprises cyclosporin A, Polyglycerol-3®, and Anise oil.

Another embodiment comprises cyclosporin A, Redicote E Series®, and Anise oil.

Another embodiment comprises cyclosporin A, Simulsol OX 1005L®, and Anise oil.

Another embodiment comprises cyclosporin A, Stanfax 567®, and Anise oil.

Another embodiment comprises cyclosporin A, TA-1618®, and Anise oil.

Another embodiment comprises cyclosporin A, Witconol H-31 A®, and Anise oil.

Another embodiment comprises cyclosporin A, an amine oxide, and Anise oil.

Another embodiment comprises cyclosporin A, AO-405®, and Anise oil.

Another embodiment comprises cyclosporin A, AO-455®, and Anise oil.

Another embodiment comprises cyclosporin A, AO 728 Special®, and Anise oil.

Another embodiment comprises cyclosporin A, Barlox 12®, and Anise oil.

Another embodiment comprises cyclosporin A, Barlox 14®, and Anise oil.

Another embodiment comprises cyclosporin A, Burcoxide Lo®, and Anise oil.

Another embodiment comprises cyclosporin A, Caloxamine LO®, and Anise oil.

Another embodiment comprises cyclosporin A, Chemoxide CAW®, and Anise oil.

Another embodiment comprises cyclosporin A, Chemoxide LM-30®, and Anise oil.

Another embodiment comprises cyclosporin A, Chemoxide LO®, and Anise oil.

Another embodiment comprises cyclosporin A, Chemoxide MO®, and Anise oil.

Another embodiment comprises cyclosporin A, Colalux CAO-35®, and Anise oil.

Another embodiment comprises cyclosporin A, Colalux LO®, and Anise oil.

Another embodiment comprises cyclosporin A, DeMox CAPO®, and Anise oil.

Another embodiment comprises cyclosporin A, DeMox CSG-30®, and Anise oil.

Another embodiment comprises cyclosporin A, DeMox LAO®, and Anise oil.

Another embodiment comprises cyclosporin A, Emcol L®, and Anise oil.

Another embodiment comprises cyclosporin A, Empigen OB®, and Anise oil.

Another embodiment comprises cyclosporin A, Empigen OS/A®, and Anise oil.

Another embodiment comprises cyclosporin A, Foamox CDO®, and Anise oil.

Another embodiment comprises cyclosporin A, Foamox DMM®, and Anise oil.

Another embodiment comprises cyclosporin A, Foamox DMS®, and Anise oil.

Another embodiment comprises cyclosporin A, Genaminox KC®, and Anise oil.

Another embodiment comprises cyclosporin A, Genaminox LA®, and Anise oil.

Another embodiment comprises cyclosporin A, Hartofoam SAO®, and Anise oil.

Another embodiment comprises cyclosporin A, Hartox DMCD®, and Anise oil.

Another embodiment comprises cyclosporin A, Lipowax DAT®, and Anise oil.

Another embodiment comprises cyclosporin A, Lipowax PB Pastilles®, and Anise oil.

Another embodiment comprises cyclosporin A, Mackamine C8®, and Anise oil.

Another embodiment comprises cyclosporin A, Mackamine C10®, and Anise oil.

Another embodiment comprises cyclosporin A, Mackamine C14®, and Anise oil.

Another embodiment comprises cyclosporin A, Mackamine CAO®, and Anise oil.

Another embodiment comprises cyclosporin A, Mackamine CO®, and Anise oil.

Another embodiment comprises cyclosporin A, Mackamine LO®, and Anise oil.

Another embodiment comprises cyclosporin A, Mackamine O2®, and Anise oil.

Another embodiment comprises cyclosporin A, Mackamine SAO®, and Anise oil.

Another embodiment comprises cyclosporin A, Mackamine SO®, and Anise oil.

Another embodiment comprises cyclosporin A, Mazox KCAO®, and Anise oil.

Another embodiment comprises cyclosporin A, Monalac MO®, and Anise oil.

Another embodiment comprises cyclosporin A, Norfox LDA®, and Anise oil.

Another embodiment comprises cyclosporin A, Rhodamox LO®, and Anise oil.

Another embodiment comprises cyclosporin A, Schercamox C-AA®, and Anise oil.

Another embodiment comprises cyclosporin A, Schercamox DMA®, and Anise oil.

Another embodiment comprises cyclosporin A, Schercamox DML®, and Anise oil.

Another embodiment comprises cyclosporin A, Schercamox DMM®, and Anise oil.

Another embodiment comprises cyclosporin A, Schercamox DMS®, and Anise oil.

Another embodiment comprises cyclosporin A, Tegotens DO®, and Anise oil.

Another embodiment comprises cyclosporin A, Tomah AO-14-2®, and Anise oil.

Another embodiment comprises cyclosporin A, Triaminox CDO®, and Anise oil.

Another embodiment comprises cyclosporin A, a block polymer, and Anise oil.

Another embodiment comprises cyclosporin A, AL 2070®, and Anise oil.

Another embodiment comprises cyclosporin A, Antarox 17-R-2®, and Anise oil.

Another embodiment comprises cyclosporin A, Antarox 25-R-2®, and Anise oil.

Another embodiment comprises cyclosporin A, Antarox 31-R-1®, and Anise oil.

Another embodiment comprises cyclosporin A, Antarox P-84®, and Anise oil.

Another embodiment comprises cyclosporin A, Antarox P-104/H®, and Anise oil.

Another embodiment comprises cyclosporin A, Arnox BP-Series®, and Anise oil.

Another embodiment comprises cyclosporin A, Chemonic 435®, and Anise oil.

Another embodiment comprises cyclosporin A, Chemonic D-25®, and Anise oil.

Another embodiment comprises cyclosporin A, Chemonic PL Series®, and Anise oil.

Another embodiment comprises cyclosporin A, Ethox L-121®, and Anise oil.

Another embodiment comprises cyclosporin A, Ethox L-122®, and Anise oil.

Another embodiment comprises cyclosporin A, Genapol PF-10®, and Anise oil.

Another embodiment comprises cyclosporin A, Genapol PF-20®, and Anise oil.

Another embodiment comprises cyclosporin A, Genapol PF-40A®, and Anise oil.

Another embodiment comprises cyclosporin A, Norfox 2-LF®, and Anise oil.

Another embodiment comprises cyclosporin A, Simulsol NW 342®, and Anise oil.

Another embodiment comprises cyclosporin A, T-Det BP-1®, and Anise oil.

Another embodiment comprises cyclosporin A, T-Det XD®, and Anise oil.

Another embodiment comprises cyclosporin A, T-Det XH®, and Anise oil.

Another embodiment comprises cyclosporin A, Triton CF-32®, and Anise oil.

Another embodiment comprises cyclosporin A, Witconol 171®, and Anise oil.

Another embodiment comprises cyclosporin A, Witconol 324®, and Anise oil.

Another embodiment comprises cyclosporin A, Witconol 324D®, and Anise oil.

Another embodiment comprises cyclosporin A, Witconol PD-2000®, and Anise oil.

Another embodiment comprises cyclosporin A, a Carboxylated Alcohol or Alkylphenol Ethoxylate, and Anise oil.

Another embodiment comprises cyclosporin A, Emcol CN-6®, and Anise oil.

Another embodiment comprises cyclosporin A, Ethcarb®, and Anise oil.

Another embodiment comprises cyclosporin A, Gemtex WNT-Conc®, and Anise oil.

Another embodiment comprises cyclosporin A, Incrodet TD7-C®, and Anise oil.

Another embodiment comprises cyclosporin A, Marlinat CM 105/80®, and Anise oil.

Another embodiment comprises cyclosporin A, Marlowet 1072®, and Anise oil.

Another embodiment comprises cyclosporin A, Marlowet 4530®, and Anise oil.

Another embodiment comprises cyclosporin A, Marlowet 4530 LF®, and Anise oil.

Another embodiment comprises cyclosporin A, Marlowet 4534®, and Anise oil.

Another embodiment comprises cyclosporin A, Marlowet 4538®, and Anise oil.

Another embodiment comprises cyclosporin A, Marlowet 4539®, and Anise oil.

Another embodiment comprises cyclosporin A, Marlowet 4539 LF®, and Anise oil.

Another embodiment comprises cyclosporin A, Marlowet 4541®, and Anise oil.

Another embodiment comprises cyclosporin A, Miranate LEC-80®, and Anise oil.

Another embodiment comprises cyclosporin A, Sandopan B®, and Anise oil.

Another embodiment comprises cyclosporin A, Sandopan B Modified®, and Anise oil.

Another embodiment comprises cyclosporin A, Sandopan LS-24 Gel®, and Anise oil.

Another embodiment comprises cyclosporin A, Surfine T-A®, and Anise oil.

Another embodiment comprises cyclosporin A, a Carboxylic Acid/Fatty Acid, and Anise oil.

Another embodiment comprises cyclosporin A, Colaterge RAM®, and Anise oil.

Another embodiment comprises cyclosporin A, Colatrope INC®, and Anise oil.

Another embodiment comprises cyclosporin A, Crodacid B®, and Anise oil.

Another embodiment comprises cyclosporin A, DeTrope CA-100®, and Anise oil.

Another embodiment comprises cyclosporin A, Latol MTO®, and Anise oil.

Another embodiment comprises cyclosporin A, Lumulse CC-33 K®, and Anise oil.

Another embodiment comprises cyclosporin A, Mulls 2218®, and Anise oil.

Another embodiment comprises cyclosporin A, OL-600®, and Anise oil.

Another embodiment comprises cyclosporin A, OL-800®, and Anise oil.

Another embodiment comprises cyclosporin A, R-910®, and Anise oil.

Another embodiment comprises cyclosporin A, S-210®, and Anise oil.

Another embodiment comprises cyclosporin A, Sandopan DTC Acid®, and Anise oil.

Another embodiment comprises cyclosporin A, Sandopan LS 24 N®, and Anise oil.

Another embodiment comprises cyclosporin A, Sandopan MA-18®, and Anise oil.

Another embodiment comprises cyclosporin A, an Ethoxylated Alcohol, and Anise oil.

Another embodiment comprises cyclosporin A, Adsee 799®, and Anise oil.

Another embodiment comprises cyclosporin A, Adsee 799®, and Anise oil.

Another embodiment comprises cyclosporin A, Alfonic 610-3.5®, and Anise oil.

Another embodiment comprises cyclosporin A, Alfonic 810-2®, and Anise oil.

Another embodiment comprises cyclosporin A, Alfonic 810-6®, and Anise oil.

Another embodiment comprises cyclosporin A, Alfonic 1012-3®, and Anise oil.

Another embodiment comprises cyclosporin A, Alfonic 1012-5®, and Anise oil.

Another embodiment comprises cyclosporin A, Alfonic 1216CO-1.5®, and Anise oil.

Another embodiment comprises cyclosporin A, Alfonic 1216CO-7®, and Anise oil.

Another embodiment comprises cyclosporin A, Alfonic 1412-3®, and Anise oil.

Another embodiment comprises cyclosporin A, Alfonic 1412-7®, and Anise oil.

Another embodiment comprises cyclosporin A, Arlasolve 200®, and Anise oil.

Another embodiment comprises cyclosporin A, Arlasolve 200 Liquid®, and Anise oil.

Another embodiment comprises cyclosporin A, Armix 180-C®, and Anise oil.

Another embodiment comprises cyclosporin A, Armix 183®, and Anise oil.

Another embodiment comprises cyclosporin A, Armul 2404®, and Anise oil.

Another embodiment comprises cyclosporin A, Armul 2404®, and Anise oil.

Another embodiment comprises cyclosporin A, Atlas EMJ-C®, and Anise oil.

Another embodiment comprises cyclosporin A, Atlas G-2109®, and Anise oil.

Another embodiment comprises cyclosporin A, Atlas G-3886®, and Anise oil.

Another embodiment comprises cyclosporin A, Atlas G-3890®, and Anise oil.

Another embodiment comprises cyclosporin A, Bio Soft E-200®, and Anise oil.

Another embodiment comprises cyclosporin A, Bio Soft E-300®, and Anise oil.

Another embodiment comprises cyclosporin A, Bio Soft E-400®, and Anise oil.

Another embodiment comprises cyclosporin A, Bio Soft EN 600®, and Anise oil.

Another embodiment comprises cyclosporin A, Bio Soft TD-400®, and Anise oil.

Another embodiment comprises cyclosporin A, Bio Soft TD-630®, and Anise oil.

Another embodiment comprises cyclosporin A, Brij 30®, and Anise oil.

Another embodiment comprises cyclosporin A, Brij 52®, and Anise oil.

Another embodiment comprises cyclosporin A, Brij 56®, and Anise oil.

Another embodiment comprises cyclosporin A, Brij 58®, and Anise oil.

Another embodiment comprises cyclosporin A, Brij 72®, and Anise oil.

Another embodiment comprises cyclosporin A, Brij 76®, and Anise oil.

Another embodiment comprises cyclosporin A, Brij 78®, and Anise oil.

Another embodiment comprises cyclosporin A, Brij 93®, and Anise oil.

Another embodiment comprises cyclosporin A, Brij 97®, and Anise oil.

Another embodiment comprises cyclosporin A, Brij 98®, and Anise oil.

Another embodiment comprises cyclosporin A, Brij 700®, and Anise oil.

Another embodiment comprises cyclosporin A, Brij 700 S®, and Anise oil.

Another embodiment comprises cyclosporin A, Brij 721®, and Anise oil.

Another embodiment comprises cyclosporin A, Brij 721 S®, and Anise oil.

Another embodiment comprises cyclosporin A, Burcoterge CDG®, and Anise oil.

Another embodiment comprises cyclosporin A, Canasol AT 600®, and Anise oil.

Another embodiment comprises cyclosporin A, Canasol AT 800®, and Anise oil.

Another embodiment comprises cyclosporin A, Canasol AT 1200®, and Anise oil.

Another embodiment comprises cyclosporin A, Canasol BJ 35®, and Anise oil.

Another embodiment comprises cyclosporin A, Canasol BJ 36®, and Anise oil.

Another embodiment comprises cyclosporin A, Canasol BJ 52®, and Anise oil.

Another embodiment comprises cyclosporin A, Canasol BJ 58®, and Anise oil.

Another embodiment comprises cyclosporin A, Canasol BJ 72®, and Anise oil.

Another embodiment comprises cyclosporin A, Canasol BJ 78®, and Anise oil.

Another embodiment comprises cyclosporin A, Canasol BJ 98®, and Anise oil.

Another embodiment comprises cyclosporin A, Canasol BJ 307®, and Anise oil.

Another embodiment comprises cyclosporin A, Cerfak 1400®, and Anise oil.

Another embodiment comprises cyclosporin A, Cetomacrogol 1000 BP®, and Anise oil.

Another embodiment comprises cyclosporin A, Chemonic C-2®, and Anise oil.

Another embodiment comprises cyclosporin A, Chemonic C-10®, and Anise oil.

Another embodiment comprises cyclosporin A, Chemonic C-20®, and Anise oil.

Another embodiment comprises cyclosporin A, Chemonic CT-12®, and Anise oil.

Another embodiment comprises cyclosporin A, Chemonic CT-20®, and Anise oil.

Another embodiment comprises cyclosporin A, Chemonic CT-30®, and Anise oil.

Another embodiment comprises cyclosporin A, Chemonic CT-55®, and Anise oil.

Another embodiment comprises cyclosporin A, Chemonic G-7®, and Anise oil.

Another embodiment comprises cyclosporin A, Chemonic G-26®, and Anise oil.

Another embodiment comprises cyclosporin A, Chemonic L-4®, and Anise oil.

Another embodiment comprises cyclosporin A, Chemonic L-7®, and Anise oil.

Another embodiment comprises cyclosporin A, Chemonic L-12®, and Anise oil.

Another embodiment comprises cyclosporin A, Chemonic L-23®, and Anise oil.

Another embodiment comprises cyclosporin A, Chemonic O-2®, and Anise oil.

Another embodiment comprises cyclosporin A, Chemonic O-5®, and Anise oil.

Another embodiment comprises cyclosporin A, Chemonic O-10®, and Anise oil.

Another embodiment comprises cyclosporin A, Chemonic O-20®, and Anise oil.

Another embodiment comprises cyclosporin A, Chemonic S-2®, and Anise oil.

Another embodiment comprises cyclosporin A, Chemonic S-10®, and Anise oil.

Another embodiment comprises cyclosporin A, Chemonic S-20®, and Anise oil.

Another embodiment comprises cyclosporin A, Colamulse FE®, and Anise oil.

Another embodiment comprises cyclosporin A, Cremophor A 20®, and Anise oil.

Another embodiment comprises cyclosporin A, Cremophor SA 2®, and Anise oil.

Another embodiment comprises cyclosporin A, Dehydol 100®, and Anise oil.

Another embodiment comprises cyclosporin A, Dehydol O-4®, and Anise oil.

Another embodiment comprises cyclosporin A, Delonic C-18®, and Anise oil.

Another embodiment comprises cyclosporin A, DeSonic 6T®, and Anise oil.

Another embodiment comprises cyclosporin A, DeSonic 9D®, and Anise oil.

Another embodiment comprises cyclosporin A, DeSonic 9T®, and Anise oil.

Another embodiment comprises cyclosporin A, DeSonic 12D®, and Anise oil.

Another embodiment comprises cyclosporin A, DeSonic 12T®, and Anise oil.

Another embodiment comprises cyclosporin A, DeSonic 15T®, and Anise oil.

Another embodiment comprises cyclosporin A, DeSonic TDA-9®, and Anise oil.

Another embodiment comprises cyclosporin A, DeThox GLG-7®, and Anise oil.

Another embodiment comprises cyclosporin A, DeThox GLG-26®, and Anise oil.

Another embodiment comprises cyclosporin A, DeThox LA-4®, and Anise oil.

Another embodiment comprises cyclosporin A, DeThox LA-23®, and Anise oil.

Another embodiment comprises cyclosporin A, DeThox SA-80®, and Anise oil.

Another embodiment comprises cyclosporin A, Disponil O5®, and Anise oil.

Another embodiment comprises cyclosporin A, Eccoterge EO-41 B®, and Anise oil.

Another embodiment comprises cyclosporin A, Empilan KA2.5/90®, and Anise oil.

Another embodiment comprises cyclosporin A, Empilan KA5/90®, and Anise oil.

Another embodiment comprises cyclosporin A, Empilan KM-20®, and Anise oil.

Another embodiment comprises cyclosporin A, Empilan KM-50®, and Anise oil.

Another embodiment comprises cyclosporin A, Empilan L-23®, and Anise oil.

Another embodiment comprises cyclosporin A, Ethylan 25-3®, and Anise oil.

Another embodiment comprises cyclosporin A, Ethylan 1204®, and Anise oil.

Another embodiment comprises cyclosporin A, Ethylan DA-4®, and Anise oil.

Another embodiment comprises cyclosporin A, Ethylan LA-230®, and Anise oil.

Another embodiment comprises cyclosporin A, Ethylan SN®, and Anise oil.

Another embodiment comprises cyclosporin A, Ethylan TD-60®, and Anise oil.

Another embodiment comprises cyclosporin A, Ethylan TD-100®, and Anise oil.

Another embodiment comprises cyclosporin A, Ethylan TD-1407®, and Anise oil.

Another embodiment comprises cyclosporin A, Eumulgin B1®, and Anise oil.

Another embodiment comprises cyclosporin A, Eumulgin B2®, and Anise oil.

Another embodiment comprises cyclosporin A, Eumulgin B3®, and Anise oil.

Another embodiment comprises cyclosporin A, Eumulgin O-10®, and Anise oil.

Another embodiment comprises cyclosporin A, Flo Mo 80/20®, and Anise oil.

Another embodiment comprises cyclosporin A, Flo Mo Low Foam®, and Anise oil.

Another embodiment comprises cyclosporin A, Forlan C-24®, and Anise oil.

Another embodiment comprises cyclosporin A, Genapol 1454®, and Anise oil.

Another embodiment comprises cyclosporin A, Genapol BA-020®, and Anise oil.

Another embodiment comprises cyclosporin A, Genapol BA-040®, and Anise oil.

Another embodiment comprises cyclosporin A, Genapol C-100®, and Anise oil.

Another embodiment comprises cyclosporin A, Genapol DA 060®, and Anise oil.

Another embodiment comprises cyclosporin A, Genapol HS 020®, and Anise oil.

Another embodiment comprises cyclosporin A, Genapol HS 200®, and Anise oil.

Another embodiment comprises cyclosporin A, Genapol ID-040®, and Anise oil.

Another embodiment comprises cyclosporin A, Genapol ID-060®, and Anise oil.

Another embodiment comprises cyclosporin A, Genapol ID-090®, and Anise oil.

Another embodiment comprises cyclosporin A, Genapol LA 010®, and Anise oil.

Another embodiment comprises cyclosporin A, Genapol LA 020®, and Anise oil.

Another embodiment comprises cyclosporin A, Genapol LA 030®, and Anise oil.

Another embodiment comprises cyclosporin A, Genapol LA 040®, and Anise oil.

Another embodiment comprises cyclosporin A, Genapol LA 050®, and Anise oil.

Another embodiment comprises cyclosporin A, Genapol LA 060®, and Anise oil.

Another embodiment comprises cyclosporin A, Genapol LA 070®, and Anise oil.

Another embodiment comprises cyclosporin A, Genapol LA 070S®, and Anise oil.

Another embodiment comprises cyclosporin A, Genapol LA 230®, and Anise oil.

Another embodiment comprises cyclosporin A, Genapol O 020®, and Anise oil.

Another embodiment comprises cyclosporin A, Genapol O 050®, and Anise oil.

Another embodiment comprises cyclosporin A, Genapol O 100®, and Anise oil.

Another embodiment comprises cyclosporin A, Genapol O 200®, and Anise oil.

Another embodiment comprises cyclosporin A, Genapol SA 030®, and Anise oil.

Another embodiment comprises cyclosporin A, Genapol SA 120®, and Anise oil.

Another embodiment comprises cyclosporin A, Genapol T-020®, and Anise oil.

Another embodiment comprises cyclosporin A, Genapol UD-030®, and Anise oil.

Another embodiment comprises cyclosporin A, Genapol UD-050®, and Anise oil.

Another embodiment comprises cyclosporin A, Genapol UD-070®, and Anise oil.

Another embodiment comprises cyclosporin A, Genapol UD-079®, and Anise oil.

Another embodiment comprises cyclosporin A, Genapol UD-080®, and Anise oil.

Another embodiment comprises cyclosporin A, Genapol UD-110®, and Anise oil.

Another embodiment comprises cyclosporin A, Genapol X 030®, and Anise oil.

Another embodiment comprises cyclosporin A, Genapol X 050®, and Anise oil.

Another embodiment comprises cyclosporin A, Genapol X 060®, and Anise oil.

Another embodiment comprises cyclosporin A, Genapol X 070®, and Anise oil.

Another embodiment comprises cyclosporin A, Genapol X 080®, and Anise oil.

Another embodiment comprises cyclosporin A, Genapol X 100®, and Anise oil.

Another embodiment comprises cyclosporin A, Genapol X159®, and Anise oil.

Another embodiment comprises cyclosporin A, Generol 122 E5®, and Anise oil.

Another embodiment comprises cyclosporin A, Generol 122 E25®, and Anise oil.

Another embodiment comprises cyclosporin A, Hostacerin T-3®, and Anise oil.

Another embodiment comprises cyclosporin A, Iconol LF 110®, and Anise oil.

Another embodiment comprises cyclosporin A, Incropol CS-20®, and Anise oil.

Another embodiment comprises cyclosporin A, Lexemul CS-20®, and Anise oil.

Another embodiment comprises cyclosporin A, Liponic EG-1®, and Anise oil.

Another embodiment comprises cyclosporin A, Lipowax D®, and Anise oil.

Another embodiment comprises cyclosporin A, Lipowax G®, and Anise oil.

Another embodiment comprises cyclosporin A, Lipowax NI®, and Anise oil.

Another embodiment comprises cyclosporin A, Lipowax P®, and Anise oil.

Another embodiment comprises cyclosporin A, Lipowax P-31®, and Anise oil.

Another embodiment comprises cyclosporin A, Lipowax PR®, and Anise oil.

Another embodiment comprises cyclosporin A, Lumulse CS-20®, and Anise oil.

Another embodiment comprises cyclosporin A, Macol CSA-20®, and Anise oil.

Another embodiment comprises cyclosporin A, Marlox B 24/50®, and Anise oil.

Another embodiment comprises cyclosporin A, Mazawet 77®, and Anise oil.

Another embodiment comprises cyclosporin A, Norfox 1713®, and Anise oil.

Another embodiment comprises cyclosporin A, Norfox 2579®, and Anise oil.

Another embodiment comprises cyclosporin A, Norfox Lo Foam®, and Anise oil.

Another embodiment comprises cyclosporin A, Promulgen D®, and Anise oil.

Another embodiment comprises cyclosporin A, Promulgen G®, and Anise oil.

Another embodiment comprises cyclosporin A, Renex 30®, and Anise oil.

Another embodiment comprises cyclosporin A, Renex 36®, and Anise oil.

Another embodiment comprises cyclosporin A, Rhodasurf A 24®, and Anise oil.

Another embodiment comprises cyclosporin A, Rhodasurf ME-10®, and Anise oil.

Another embodiment comprises cyclosporin A, Rhodasurf BEH-25®, and Anise oil.

Another embodiment comprises cyclosporin A, Rhodasurf BEH-40®, and Anise oil.

Another embodiment comprises cyclosporin A, Rhodasurf DA 530®, and Anise oil.

Another embodiment comprises cyclosporin A, Rhodasurf DA 630®, and Anise oil.

Another embodiment comprises cyclosporin A, Rhodasurf DA 639®, and Anise oil.

Another embodiment comprises cyclosporin A, Rhodasurf LAN-23®, and Anise oil.

Another embodiment comprises cyclosporin A, Rhodasurf ON-870®, and Anise oil.

Another embodiment comprises cyclosporin A, Rhodasurf ON-877®, and Anise oil.

Another embodiment comprises cyclosporin A, Rhodasurf TB-970 FLK®, and Anise oil.

Another embodiment comprises cyclosporin A, Ritacet-20®, and Anise oil.

Another embodiment comprises cyclosporin A, Ritachol 1000®, and Anise oil.

Another embodiment comprises cyclosporin A, Ritachol 2000®, and Anise oil.

Another embodiment comprises cyclosporin A, Ritachol 5000®, and Anise oil.

Another embodiment comprises cyclosporin A, Ritox 35®, and Anise oil.

Another embodiment comprises cyclosporin A, Surfonic DA-4®, and Anise oil.

Another embodiment comprises cyclosporin A, Surfonic DA-6®, and Anise oil.

Another embodiment comprises cyclosporin A, Surfonic L46-7®, and Anise oil.

Another embodiment comprises cyclosporin A, Surfonic POA®, and Anise oil.

Another embodiment comprises cyclosporin A, Synthrapol KB®, and Anise oil.

Another embodiment comprises cyclosporin A, Teginacid®, and Anise oil.

Another embodiment comprises cyclosporin A, Teginacid C®, and Anise oil.

Another embodiment comprises cyclosporin A, Tegotens EC 11®, and Anise oil.

Another embodiment comprises cyclosporin A, Tinegal NA®, and Anise oil.

Another embodiment comprises cyclosporin A, Tomadol 400®, and Anise oil.

Another embodiment comprises cyclosporin A, Tomadol 600®, and Anise oil.

Another embodiment comprises cyclosporin A, Tomadol 900®, and Anise oil.

Another embodiment comprises cyclosporin A, Uniperol O®, and Anise oil.

Another embodiment comprises cyclosporin A, Witconol SN Series®, and Anise oil.

Another embodiment comprises cyclosporin A, an Ethoxylated Alkylphenol, and Anise oil.

Another embodiment comprises cyclosporin A, Antarox LF-222®, and Anise oil.

Another embodiment comprises cyclosporin A, Atlox 775®, and Anise oil.

Another embodiment comprises cyclosporin A, Caloxylate N-9®, and Anise oil.

Another embodiment comprises cyclosporin A, Canasol NF-1000®, and Anise oil.

Another embodiment comprises cyclosporin A, Canasol NF-3000®, and Anise oil.

Another embodiment comprises cyclosporin A, Canasol NF-3070®, and Anise oil.

Another embodiment comprises cyclosporin A, Canasol OF 1670®, and Anise oil.

Another embodiment comprises cyclosporin A, Canasol OF 2570®, and Anise oil.

Another embodiment comprises cyclosporin A, Canasol OF 4070®, and Anise oil.

Another embodiment comprises cyclosporin A, Chemax DNP-8®, and Anise oil.

Another embodiment comprises cyclosporin A, Chemax DNP-18®, and Anise oil.

Another embodiment comprises cyclosporin A, Chemax DNP-150/50®, and Anise oil.

Another embodiment comprises cyclosporin A, DeSonic 1.5N®, and Anise oil.

Another embodiment comprises cyclosporin A, DeSonic 4N®, and Anise oil.

Another embodiment comprises cyclosporin A, DeSonic 5N®, and Anise oil.

Another embodiment comprises cyclosporin A, DeSonic 6D®, and Anise oil.

Another embodiment comprises cyclosporin A, DeSonic 6N®, and Anise oil.

Another embodiment comprises cyclosporin A, DeSonic 7N®, and Anise oil.

Another embodiment comprises cyclosporin A, DeSonic 9N®, and Anise oil.

Another embodiment comprises cyclosporin A, DeSonic 10D®, and Anise oil.

Another embodiment comprises cyclosporin A, DeSonic 11N®, and Anise oil.

Another embodiment comprises cyclosporin A, DeSonic 12N®, and Anise oil.

Another embodiment comprises cyclosporin A, DeSonic 15N®, and Anise oil.

Another embodiment comprises cyclosporin A, DeSonic 20N®, and Anise oil.

Another embodiment comprises cyclosporin A, Eccoscour RC®, and Anise oil.

Another embodiment comprises cyclosporin A, Eccoterge EO-100®, and Anise oil.

Another embodiment comprises cyclosporin A, Emulsifier 632/90%®, and Anise oil.

Another embodiment comprises cyclosporin A, Geronol AG-821®, and Anise oil.

Another embodiment comprises cyclosporin A, Gradonic N-95®, and Anise oil.

Another embodiment comprises cyclosporin A, Hetoxide NP-4®, and Anise oil.

Another embodiment comprises cyclosporin A, Hetoxide NP-30®, and Anise oil.

Another embodiment comprises cyclosporin A, Hostapal N-100®, and Anise oil.

Another embodiment comprises cyclosporin A, Hostapal N-110®, and Anise oil.

Another embodiment comprises cyclosporin A, Igepal CTA-639W®, and Anise oil.

Another embodiment comprises cyclosporin A, Igepal DAP-9®, and Anise oil.

Another embodiment comprises cyclosporin A, Igepal OD-410®, and Anise oil.

Another embodiment comprises cyclosporin A, Igepal SS-837®, and Anise oil.

Another embodiment comprises cyclosporin A, Lipocol NP-9 USP®, and Anise oil.

Another embodiment comprises cyclosporin A, Macol DNP-10®, and Anise oil.

Another embodiment comprises cyclosporin A, Marlophen NP 5®, and Anise oil.

Another embodiment comprises cyclosporin A, Marlophen P 1®, and Anise oil.

Another embodiment comprises cyclosporin A, Surfonic NB®, and Anise oil.

Another embodiment comprises cyclosporin A, Surfonic OPB-307®, and Anise oil.

Another embodiment comprises cyclosporin A, Surfonic OPB-407®, and Anise oil.

Another embodiment comprises cyclosporin A, Syn Fac 334®, and Anise oil.

Another embodiment comprises cyclosporin A, Syn Fac 8216®, and Anise oil.

Another embodiment comprises cyclosporin A, Triton N-57®, and Anise oil.

Another embodiment comprises cyclosporin A, Trycol 6956®, and Anise oil.

Another embodiment comprises cyclosporin A, Trycol 6961®, and Anise oil.

Another embodiment comprises cyclosporin A, Trycol 6964®, and Anise oil.

Another embodiment comprises cyclosporin A, Trycol 6969®, and Anise oil.

Another embodiment comprises cyclosporin A, Trycol 6974®, and Anise oil.

Another embodiment comprises cyclosporin A, Witbreak DRB-127®, and Anise oil.

Another embodiment comprises cyclosporin A, Witbreak DRB-127®, and Anise oil.

Another embodiment comprises cyclosporin A, Witconol NP Series®, and Anise oil.

Another embodiment comprises cyclosporin A, an Ethoxylated Aryl Phenol, and Anise oil.

Another embodiment comprises cyclosporin A, Soprophor BSU®, and Anise oil.

Another embodiment comprises cyclosporin A, Soprophor CY/8®, and Anise oil.

Another embodiment comprises cyclosporin A, Soprophor S/25®, and Anise oil.

Another embodiment comprises cyclosporin A, Witconol NIO®, and Anise oil.

Another embodiment comprises cyclosporin A, Witconol NIW®, and Anise oil.

Another embodiment comprises cyclosporin A, Witconol S-100®, and Anise oil.

Another embodiment comprises cyclosporin A, an Ethoxylated Fatty Acid, and Anise oil.

Another embodiment comprises cyclosporin A, Aldo PGHMS®, and Anise oil.

Another embodiment comprises cyclosporin A, Alkamuls TO-15/HR®, and Anise oil.

Another embodiment comprises cyclosporin A, Armotan AL-69-66®, and Anise oil.

Another embodiment comprises cyclosporin A, Cerasynt 840®, and Anise oil.

Another embodiment comprises cyclosporin A, Cerasynt 945®, and Anise oil.

Another embodiment comprises cyclosporin A, Crystal Inhibitor No. 5®, and Anise oil.

Another embodiment comprises cyclosporin A, DeThox Acid L-9®, and Anise oil.

Another embodiment comprises cyclosporin A, DeThox Acid S-8®, and Anise oil.

Another embodiment comprises cyclosporin A, Ethofat 242/25®, and Anise oil.

Another embodiment comprises cyclosporin A, Hydropalat 65®, and Anise oil.

Another embodiment comprises cyclosporin A, Lipo EGMS®, and Anise oil.

Another embodiment comprises cyclosporin A, Lipopeg 2 DL®, and Anise oil.

Another embodiment comprises cyclosporin A, Lipopeg 4 DL®, and Anise oil.

Another embodiment comprises cyclosporin A, Lipopeg 4-L®, and Anise oil.

Another embodiment comprises cyclosporin A, Lipopeg 39-S®, and Anise oil.

Another embodiment comprises cyclosporin A, Lipopeg 4-S®, and Anise oil.

Another embodiment comprises cyclosporin A, Lipopeg 10-S®, and Anise oil.

Another embodiment comprises cyclosporin A, Lipopeg 100-S®, and Anise oil.

Another embodiment comprises cyclosporin A, Lipopeg 6000 DS®, and Anise oil.

Another embodiment comprises cyclosporin A, Lumulse 40-L®, and Anise oil.

Another embodiment comprises cyclosporin A, Lumulse 40-S®, and Anise oil.

Another embodiment comprises cyclosporin A, Lumulse 42-L®, and Anise oil.

Another embodiment comprises cyclosporin A, Lumulse 42-S®, and Anise oil.

Another embodiment comprises cyclosporin A, Lumulse 100-S®, and Anise oil.

Another embodiment comprises cyclosporin A, Lumulse 602-S®, and Anise oil.

Another embodiment comprises cyclosporin A, Magrabar PGE-20-0®, and Anise oil.

Another embodiment comprises cyclosporin A, Magrabar PGE-20L®, and Anise oil.

Another embodiment comprises cyclosporin A, Magrabar PGE-20T®, and Anise oil.

Another embodiment comprises cyclosporin A, Magrabar PGE-22-0®, and Anise oil.

Another embodiment comprises cyclosporin A, Magrabar PGE-22L®, and Anise oil.

Another embodiment comprises cyclosporin A, Magrabar PGE-22T®, and Anise oil.

Another embodiment comprises cyclosporin A, Magrabar PGE-40-0®, and Anise oil.

Another embodiment comprises cyclosporin A, Magrabar PGE-40L®, and Anise oil.

Another embodiment comprises cyclosporin A, Magrabar PGE-40T®, and Anise oil.

Another embodiment comprises cyclosporin A, Magrabar PGE-42-0®, and Anise oil.

Another embodiment comprises cyclosporin A, Magrabar PGE-42L®, and Anise oil.

Another embodiment comprises cyclosporin A, Magrabar PGE-42T®, and Anise oil.

Another embodiment comprises cyclosporin A, Magrabar PGE-60-0®, and Anise oil.

Another embodiment comprises cyclosporin A, Magrabar PGE-60L®, and Anise oil.

Another embodiment comprises cyclosporin A, Magrabar PGE-60T®, and Anise oil.

Another embodiment comprises cyclosporin A, Magrabar PGE-62-0®, and Anise oil.

Another embodiment comprises cyclosporin A, Magrabar PGE-62L®, and Anise oil.

Another embodiment comprises cyclosporin A, Magrabar PGE-62T®, and Anise oil.

Another embodiment comprises cyclosporin A, Mapeg S-40K®, and Anise oil.

Another embodiment comprises cyclosporin A, Marlowet OTS®, and Anise oil.

Another embodiment comprises cyclosporin A, Naturechem PGR®, and Anise oil.

Another embodiment comprises cyclosporin A, PG No. 4®, and Anise oil.

Another embodiment comprises cyclosporin A, Renex 20®, and Anise oil.

Another embodiment comprises cyclosporin A, Ritox 52®, and Anise oil.

Another embodiment comprises cyclosporin A, Ritox 53®, and Anise oil.

Another embodiment comprises cyclosporin A, Ritox 59®, and Anise oil.

Another embodiment comprises cyclosporin A, Surfax 8916/A®, and Anise oil.

Another embodiment comprises cyclosporin A, Tego Acid S 40 P®, and Anise oil.

Another embodiment comprises cyclosporin A, Tego Acid S 100 P®, and Anise oil.

Another embodiment comprises cyclosporin A, Tween 20®, and Anise oil.

Another embodiment comprises cyclosporin A, Volpo 131®, and Anise oil.

Another embodiment comprises cyclosporin A, an Ethoxylated Fatty Ester or Oil, and Anise oil.

Another embodiment comprises cyclosporin A, Acconon 6-C10®, and Anise oil.

Another embodiment comprises cyclosporin A, Acconon CC-6®, and Anise oil.

Another embodiment comprises cyclosporin A, Acconon CO-7®, and Anise oil.

Another embodiment comprises cyclosporin A, Aldosperse 40/60 FG®, and Anise oil.

Another embodiment comprises cyclosporin A, Aldosperse ML-23®, and Anise oil.

Another embodiment comprises cyclosporin A, Aldosperse MS-20 FG®, and Anise oil.

Another embodiment comprises cyclosporin A, Alkamuls EL-620®, and Anise oil.

Another embodiment comprises cyclosporin A, Alkamuls EL-719®, and Anise oil.

Another embodiment comprises cyclosporin A, Alkamuls EL-985®, and Anise oil.

Another embodiment comprises cyclosporin A, Arlatone G®, and Anise oil.

Another embodiment comprises cyclosporin A, Arlatone T®, and Anise oil.

Another embodiment comprises cyclosporin A, Atlas G-1045A®, and Anise oil.

Another embodiment comprises cyclosporin A, Atlas G-1086®, and Anise oil.

Another embodiment comprises cyclosporin A, Atlas G-1087®, and Anise oil.

Another embodiment comprises cyclosporin A, Atlas G-1089®, and Anise oil.

Another embodiment comprises cyclosporin A, Atlas G-1096®, and Anise oil.

Another embodiment comprises cyclosporin A, Atlas G-1292®, and Anise oil.

Another embodiment comprises cyclosporin A, Atlas G-1293®, and Anise oil.

Another embodiment comprises cyclosporin A, Atlas G-1300®, and Anise oil.

Another embodiment comprises cyclosporin A, Atlas G-7076®, and Anise oil.

Another embodiment comprises cyclosporin A, Capmul EMG®, and Anise oil.

Another embodiment comprises cyclosporin A, Chemonic CO-40®, and Anise oil.

Another embodiment comprises cyclosporin A, Chemonic LI-3®, and Anise oil.

Another embodiment comprises cyclosporin A, Chemonic LI-7®, and Anise oil.

Another embodiment comprises cyclosporin A, Cirrasol GM®, and Anise oil.

Another embodiment comprises cyclosporin A, Cremophor CO 40®, and Anise oil.

Another embodiment comprises cyclosporin A, Cremophor CO 410®, and Anise oil.

Another embodiment comprises cyclosporin A, Cremophor EL®, and Anise oil.

Another embodiment comprises cyclosporin A, Cremophor GC7®, and Anise oil.

Another embodiment comprises cyclosporin A, Cremophor RH-40®, and Anise oil.

Another embodiment comprises cyclosporin A, Crovol A-40®, and Anise oil.

Another embodiment comprises cyclosporin A, Crovol A-70®, and Anise oil.

Another embodiment comprises cyclosporin A, Crovol M-70®, and Anise oil.

Another embodiment comprises cyclosporin A, Crovol PK-70®, and Anise oil.

Another embodiment comprises cyclosporin A, Cutina E-24®, and Anise oil.

Another embodiment comprises cyclosporin A, Dacospin 12-R®, and Anise oil.

Another embodiment comprises cyclosporin A, Dehymuls HRE-7®, and Anise oil.

Another embodiment comprises cyclosporin A, DeSonic 30C®, and Anise oil.

Another embodiment comprises cyclosporin A, DeSonic 36C®, and Anise oil.

Another embodiment comprises cyclosporin A, DeSonic 40C®, and Anise oil.

Another embodiment comprises cyclosporin A, Durfax 60®, and Anise oil.

Another embodiment comprises cyclosporin A, Durfax 65®, and Anise oil.

Another embodiment comprises cyclosporin A, Durfax 80®, and Anise oil.

Another embodiment comprises cyclosporin A, Durfax EOM®, and Anise oil.

Another embodiment comprises cyclosporin A, Eccoterge NF-2®, and Anise oil.

Another embodiment comprises cyclosporin A, Emulpon CO-360®, and Anise oil.

Another embodiment comprises cyclosporin A, Emulpon CO-550®, and Anise oil.

Another embodiment comprises cyclosporin A, Emulsogen EL®, and Anise oil.

Another embodiment comprises cyclosporin A, Emulsogen HCO 040®, and Anise oil.

Another embodiment comprises cyclosporin A, Emulsogen HCO 060®, and Anise oil.

Another embodiment comprises cyclosporin A, Emulsynt 1055®, and Anise oil.

Another embodiment comprises cyclosporin A, Ethox 3095®, and Anise oil.

Another embodiment comprises cyclosporin A, Eumulgin RO-40®, and Anise oil.

Another embodiment comprises cyclosporin A, Genapol G-260®, and Anise oil.

Another embodiment comprises cyclosporin A, Glycosperse L-20®, and Anise oil.

Another embodiment comprises cyclosporin A, Glycosperse O-5®, and Anise oil.

Another embodiment comprises cyclosporin A, Glycosperse O-20®, and Anise oil.

Another embodiment comprises cyclosporin A, Glycosperse O-20 FG®, and Anise oll.

Another embodiment comprises cyclosporin A, Glycosperse S-20®, and Anise oil.

Another embodiment comprises cyclosporin A, Glycosperse S-20 FG®, and Anise oil.

Another embodiment comprises cyclosporin A, Glycosperse TS-20®, and Anise oil.

Another embodiment comprises cyclosporin A, Glycosperse TS-20 FG®, and Anise oil.

Another embodiment comprises cyclosporin A, Hetan SL®, and Anise oil.

Another embodiment comprises cyclosporin A, Hetan SO®, and Anise oil.

Another embodiment comprises cyclosporin A, Hetan SS®, and Anise oil.

Another embodiment comprises cyclosporin A, Hetoxide C-2®, and Anise oil.

Another embodiment comprises cyclosporin A, Hetoxide C-9®, and Anise oil.

Another embodiment comprises cyclosporin A, Hetoxide C-15®, and Anise oil.

Another embodiment comprises cyclosporin A, Hetoxide C-25®, and Anise oil.

Another embodiment comprises cyclosporin A, Hetoxide C-40®, and Anise oil.

Another embodiment comprises cyclosporin A, Hetoxide C-200®, and Anise oil.

Another embodiment comprises cyclosporin A, Hetoxide C-200-50%®, and Anise oil.

Another embodiment comprises cyclosporin A, Hetoxide GC-30®, and Anise oil.

Another embodiment comprises cyclosporin A, Hetoxide HC-60®, and Anise oil.

Another embodiment comprises cyclosporin A, Ice No. 2®, and Anise oil.

Another embodiment comprises cyclosporin A, Incrocas 30/40®, and Anise oil.

Another embodiment comprises cyclosporin A, Lexol EC®, and Anise oil.

Another embodiment comprises cyclosporin A, Lexol EO®, and Anise oil.

Another embodiment comprises cyclosporin A, Lipocol HCO-40®, and Anise oil.

Another embodiment comprises cyclosporin A, Lipocol HCO-60®, and Anise oil.

Another embodiment comprises cyclosporin A, Lipocol O-3 Special®, and Anise oil.

Another embodiment comprises cyclosporin A, Lipopeg 2-L®, and Anise oil.

Another embodiment comprises cyclosporin A, Lipopeg 4-DO®, and Anise oil.

Another embodiment comprises cyclosporin A, Lipopeg 4-DS®, and Anise oil.

Another embodiment comprises cyclosporin A, Lipovol GTB®, and Anise oil.

Another embodiment comprises cyclosporin A, Lonzest SML-20®, and Anise oil.

Another embodiment comprises cyclosporin A, Lonzest SMO-20®, and Anise oil.

Another embodiment comprises cyclosporin A, Lonzest SMS-20®, and Anise oil.

Another embodiment comprises cyclosporin A, Lonzest STO-20®, and Anise oil.

Another embodiment comprises cyclosporin A, Lonzest STS-20®, and Anise oil.

Another embodiment comprises cyclosporin A, Lumulse GR-40®, and Anise oil.

Another embodiment comprises cyclosporin A, Lumulse GRH-40®, and Anise oil.

Another embodiment comprises cyclosporin A, Lumulse POE (7) GML®, and Anise oil.

Another embodiment comprises cyclosporin A, Lumulse POE (12) Glyc®, and Anise oil.

Another embodiment comprises cyclosporin A, Lumulse POE (40) MS KP®, and Anise oil.

Another embodiment comprises cyclosporin A, Marlowet 4750®, and Anise oil.

Another embodiment comprises cyclosporin A, Marlowet LVS®, and Anise oil.

Another embodiment comprises cyclosporin A, Marlowet R 11®, and Anise oil.

Another embodiment comprises cyclosporin A, Marlowet R 40®, and Anise oil.

Another embodiment comprises cyclosporin A, Mazol 80 MGK®, and Anise oil.

Another embodiment comprises cyclosporin A, Nonionic Emulsifier T-9®, and Anise oil.

Another embodiment comprises cyclosporin A, Oronal LCG®, and Anise oil.

Another embodiment comprises cyclosporin A, Polyderm PPI-CO-200®, and Anise oil.

Another embodiment comprises cyclosporin A, Polyderm PPI-CO-40®, and Anise oil.

Another embodiment comprises cyclosporin A, Rewoderm LI 520-70®, and Anise oil.

Another embodiment comprises cyclosporin A, Ritapeg 150 DS®, and Anise oil.

Another embodiment comprises cyclosporin A, Softigen 767®, and Anise oil.

Another embodiment comprises cyclosporin A, Surfactol 318®, and Anise oil.

Another embodiment comprises cyclosporin A, Surfactol 365®, and Anise oil.

Another embodiment comprises cyclosporin A, Syn Lube 107®, and Anise oil.

Another embodiment comprises cyclosporin A, Syn Lube 728®, and Anise oil.

Another embodiment comprises cyclosporin A, Syn Lube 1632H®, and Anise oil.

Another embodiment comprises cyclosporin A, Syn Lube 6277-A®, and Anise oil.

Another embodiment comprises cyclosporin A, T-Det C-20®, and Anise oil.

Another embodiment comprises cyclosporin A, T-Det C-40®, and Anise oil.

Another embodiment comprises cyclosporin A, Tally 100 Plus®, and Anise oil.

Another embodiment comprises cyclosporin A, Uniperol EL®, and Anise oil.

Another embodiment comprises cyclosporin A, a Fatty Ester, and Anise oil.

Another embodiment comprises cyclosporin A, Actralube-Syn 147®, and Anise oil.

Another embodiment comprises cyclosporin A, Atlas G-1556®, and Anise oil.

Another embodiment comprises cyclosporin A, Atlas G-1564®, and Anise oil.

Another embodiment comprises cyclosporin A, Atlasol Base Oil S®, and Anise oil.

Another embodiment comprises cyclosporin A, Base ML®, and Anise oil.

Another embodiment comprises cyclosporin A, Base MT®, and Anise oil.

Another embodiment comprises cyclosporin A, Cerasynt 303®, and Anise oil.

Another embodiment comprises cyclosporin A, Dermol 1012®, and Anise oil.

Another embodiment comprises cyclosporin A, Kemester 4000®, and Anise oil.

Another embodiment comprises cyclosporin A, Lactipol S®, and Anise oil.

Another embodiment comprises cyclosporin A, Magrabar PGO®, and Anise oil.

Another embodiment comprises cyclosporin A, Mayco Base BFO®, and Anise oil.

Another embodiment comprises cyclosporin A, Methyl Linoleate®, and Anise oil.

Another embodiment comprises cyclosporin A, Pationic 122A®, and Anise oil.

Another embodiment comprises cyclosporin A, Pationic 138C®, and Anise oil.

Another embodiment comprises cyclosporin A, Pationic CSL®, and Anise oil.

Another embodiment comprises cyclosporin A, Pationic ISL®, and Anise oil.

Another embodiment comprises cyclosporin A, Pationic SBL®, and Anise oil.

Another embodiment comprises cyclosporin A, Pationic SSL®, and Anise oil.

Another embodiment comprises cyclosporin A, Ritasol®, and Anise oil.

Another embodiment comprises cyclosporin A, Tego Alkanol CS 20®, and Anise oil.

Another embodiment comprises cyclosporin A, Tego Alkanol L23 P®, and Anise oil.

Another embodiment comprises cyclosporin A, Tego Alkanol S2®, and Anise oil.

Another embodiment comprises cyclosporin A, Tego Alkanol S20 P®, and Anise oil.

Another embodiment comprises cyclosporin A, Triemulsifier 600 MS®, and Anise oil.

Another embodiment comprises cyclosporin A, a Glycerol Esters, and Anise oil.

Another embodiment comprises cyclosporin A, Agro #9 Wint SBO®, and Anise oil.

Another embodiment comprises cyclosporin A, Ahcovel Base 700®, and Anise oil.

Another embodiment comprises cyclosporin A, Aldo HMS FG®, and Anise oil.

Another embodiment comprises cyclosporin A, Aldo MLD®, and Anise oil.

Another embodiment comprises cyclosporin A, Aldo MLD FG®, and Anise oil.

Another embodiment comprises cyclosporin A, Aldo MO FG®, and Anise oil.

Another embodiment comprises cyclosporin A, Aldo MS®, and Anise oil.

Another embodiment comprises cyclosporin A, Aldo MS FG®, and Anise oil.

Another embodiment comprises cyclosporin A, Aldo MS LG FG®, and Anise oil.

Another embodiment comprises cyclosporin A, Aldo MSD®, and Anise oil.

Another embodiment comprises cyclosporin A, Aldo MSD FG®, and Anise oil.

Another embodiment comprises cyclosporin A, Aldosperse O-20 FG®, and Anise oil.

Another embodiment comprises cyclosporin A, Aldosperse TS-20 FG®, and Anise oil.

Another embodiment comprises cyclosporin A, Aldosperse TS-40 FG®, and Anise oil.

Another embodiment comprises cyclosporin A, Arlacel 165®, and Anise oil.

Another embodiment comprises cyclosporin A, Arlacel 186®, and Anise oil.

Another embodiment comprises cyclosporin A, Capmul GMO®, and Anise oil.

Another embodiment comprises cyclosporin A, Capmul GMS®, and Anise oil.

Another embodiment comprises cyclosporin A, Caprol 3GO®, and Anise oil.

Another embodiment comprises cyclosporin A, Caprol 3GVS®, and Anise oil.

Another embodiment comprises cyclosporin A, Caprol 6G2S®, and Anise oil.

Another embodiment comprises cyclosporin A, Caprol 1 OG40®, and Anise oil.

Another embodiment comprises cyclosporin A, Caprol 1OG100®, and Anise oil.

Another embodiment comprises cyclosporin A, Caprol ET®, and Anise oil.

Another embodiment comprises cyclosporin A, Caprol PGE860®, and Anise oil.

Another embodiment comprises cyclosporin A, Cerasynt 945®, and Anise oil.

Another embodiment comprises cyclosporin A, Cerasynt GMS®, and Anise oil.

Another embodiment comprises cyclosporin A, Cerasynt Q®, and Anise oil.

Another embodiment comprises cyclosporin A, Cerasynt SD®, and Anise oil.

Another embodiment comprises cyclosporin A, Cerasynt WM®, and Anise oil.

Another embodiment comprises cyclosporin A, Chemsperse 14®, and Anise oil.

Another embodiment comprises cyclosporin A, Cremophor GO-32®, and Anise oil.

Another embodiment comprises cyclosporin A, Cremophor GS1 1®, and Anise oil.

Another embodiment comprises cyclosporin A, Cremophor GS-32®, and Anise oil.

Another embodiment comprises cyclosporin A, Cutina KD-16®, and Anise oil.

Another embodiment comprises cyclosporin A, Dehymuls PGPH®, and Anise oil.

Another embodiment comprises cyclosporin A, Dermol DGDIS®, and Anise oil.

Another embodiment comprises cyclosporin A, Dermol DGMIS®, and Anise oil.

Another embodiment comprises cyclosporin A, Dermol G-76®, and Anise oil.

Another embodiment comprises cyclosporin A, Dermol G-7DI®, and Anise oil.

Another embodiment comprises cyclosporin A, Dermol NGDI®, and Anise oil.

Another embodiment comprises cyclosporin A, Dermolan GLH®, and Anise oil.

Another embodiment comprises cyclosporin A, Drewmulse GMO®, and Anise oil.

Another embodiment comprises cyclosporin A, Drewpol 3-5-M®, and Anise oil.

Another embodiment comprises cyclosporin A, Durlac 100 W®, and Anise oil.

Another embodiment comprises cyclosporin A, Dur-Lo®, and Anise oil.

Another embodiment comprises cyclosporin A, Dynasan 118®, and Anise oil.

Another embodiment comprises cyclosporin A, EC-25®, and Anise oil.

Another embodiment comprises cyclosporin A, EM 40®, and Anise oil.

Another embodiment comprises cyclosporin A, Emerest 2400®, and Anise oil.

Another embodiment comprises cyclosporin A, Emerest 2452®, and Anise oil.

Another embodiment comprises cyclosporin A, Empilan G-26®, and Anise oil.

Another embodiment comprises cyclosporin A, Genapol TSM®, and Anise oil.

Another embodiment comprises cyclosporin A, Hostacerin DGI®, and Anise oil.

Another embodiment comprises cyclosporin A, Hostacerin DGL®, and Anise oil.

Another embodiment comprises cyclosporin A, Hostacerin DGMS®, and Anise oil.

Another embodiment comprises cyclosporin A, Hostacerin DGSB®, and Anise oil.

Another embodiment comprises cyclosporin A, Ice No. 2®, and Anise oil.

Another embodiment comprises cyclosporin A, Imwitor 742®, and Anise oil.

Another embodiment comprises cyclosporin A, Imwitor 780 K®, and Anise oil.

Another embodiment comprises cyclosporin A, Imwitor 960 Flakes®, and Anise oil.

Another embodiment comprises cyclosporin A, Isolan GI 34®, and Anise oil.

Another embodiment comprises cyclosporin A, Isolan GO 33®, and Anise oil.

Another embodiment comprises cyclosporin A, Kemester 1000®, and Anise oil.

Another embodiment comprises cyclosporin A, Kemester 2000®, and Anise oil.

Another embodiment comprises cyclosporin A, Kemester 2000®, and Anise oil.

Another embodiment comprises cyclosporin A, Kemester 6000SE®, and Anise oil.

Another embodiment comprises cyclosporin A, Lamecreme DGE 18®, and Anise oil.

Another embodiment comprises cyclosporin A, Lexemul 515®, and Anise oil.

Another embodiment comprises cyclosporin A, Lexemul 561®, and Anise oil.

Another embodiment comprises cyclosporin A, Lexemul AR®, and Anise oil.

Another embodiment comprises cyclosporin A, Lexemul AS®, and Anise oil.

Another embodiment comprises cyclosporin A, Lexemul GDL®, and Anise oil.

Another embodiment comprises cyclosporin A, Lexemul T®, and Anise oil.

Another embodiment comprises cyclosporin A, Lipomulse 165®, and Anise oil.

Another embodiment comprises cyclosporin A, Lumulse GML K®, and Anise oil.

Another embodiment comprises cyclosporin A, Lumulse GMO K®, and Anise oil.

Another embodiment comprises cyclosporin A, Lumulse GMR K®, and Anise oil.

Another embodiment comprises cyclosporin A, Lumulse GMT K®, and Anise oil.

Another embodiment comprises cyclosporin A, Magrabar GMC®, and Anise oil.

Another embodiment comprises cyclosporin A, Magrabar GMO-CK®, and Anise oil.

Another embodiment comprises cyclosporin A, Magrabar MDG-5050®, and Anise oil.

Another embodiment comprises cyclosporin A, Magrabar PGO-315®, and Anise oil.

Another embodiment comprises cyclosporin A, Magrabar PGO-1010®, and Anise oil.

Another embodiment comprises cyclosporin A, Mazol 300K®, and Anise oil.

Another embodiment comprises cyclosporin A, Mazol GMO-K®, and Anise oil.

Another embodiment comprises cyclosporin A, Mazol GMS-K®, and Anise oil.

Another embodiment comprises cyclosporin A, Mazol PGO31-K®, and Anise oil.

Another embodiment comprises cyclosporin A, Miglyol 812®, and Anise oil.

Another embodiment comprises cyclosporin A, Norfox 1 65C®, and Anise oil.

Another embodiment comprises cyclosporin A, Schercemol GMIS®, and Anise oil.

Another embodiment comprises cyclosporin A, Tegin®, and Anise oil.

Another embodiment comprises cyclosporin A, Tegin 4100 Pellets®, and Anise oil.

Another embodiment comprises cyclosporin A, Tegin M Pellets®, and Anise oil.

Another embodiment comprises cyclosporin A, Tegin OV®, and Anise oil.

Another embodiment comprises cyclosporin A, Teginacid H®, and Anise oil.

Another embodiment comprises cyclosporin A, Tego Cosmo P813®, and Anise oil.

Another embodiment comprises cyclosporin A, Wickenol 535®, and Anise oil.

Another embodiment comprises cyclosporin A, Witconol 14®, and Anise oil.

Another embodiment comprises cyclosporin A, Witconol 14®, and Anise oil.

Another embodiment comprises cyclosporin A, Witconol 14F®, and Anise oil.

Another embodiment comprises cyclosporin A, Witconol 18L®, and Anise oil.

Another embodiment comprises cyclosporin A, Witconol GOT®, and Anise oil.

Another embodiment comprises cyclosporin A, Witconol MST®, and Anise oil.

Another embodiment comprises cyclosporin A, Witconol RHT®, and Anise oil.

Another embodiment comprises cyclosporin A, a Glycol Ester, and Anise oil.

Another embodiment comprises cyclosporiri A, Alkamuls 600 DO®, and Anise oil.

Another embodiment comprises cyclosporin A, Alkamuls SEG®, and Anise oil.

Another embodiment comprises cyclosporin A, Atlas EM-2®, and Anise oil.

Another embodiment comprises cyclosporin A, Cerasynt IP®, and Anise oil.

Another embodiment comprises cyclosporin A, Cerasynt M®, and Anise oil.

Another embodiment comprises cyclosporin A, Cerasynt MN®, and Anise oil.

Another embodiment comprises cyclosporin A, Cerasynt PA®, and Anise oil.

Another embodiment comprises cyclosporin A, Chemsperse EGDS®, and Anise oil.

Another embodiment comprises cyclosporin A, Chemsperse EGMS®, and Anise oil.

Another embodiment comprises cyclosporin A, Colonial Monolaurin®, and Anise oil.

Another embodiment comprises cyclosporin A, DeMuls SGE-95®, and Anise oil.

Another embodiment comprises cyclosporin A, Eccoterge 200®, and Anise oil.

Another embodiment comprises cyclosporin A, Emerest 2380®, and Anise oil.

Another embodiment comprises cyclosporin A, Ethox 2610®, and Anise oil.

Another embodiment comprises cyclosporin A, Ethox DO-9®, and Anise oil.

Another embodiment comprises cyclosporin A, Ethox DO-14®, and Anise oil.

Another embodiment comprises cyclosporin A, Ethox SO-9®, and Anise oil.

Another embodiment comprises cyclosporin A, Fizul MD-318®, and Anise oil.

Another embodiment comprises cyclosporin A, Genapol EGDS-VHP®, and Anise oil.

Another embodiment comprises cyclosporin A, Genapol TS Powder®, and Anise oil.

Another embodiment comprises cyclosporin A, Hostacerin WO®, and Anise oil.

Another embodiment comprises cyclosporin A, Inversol 140®, and Anise oil.

Another embodiment comprises cyclosporin A, Kemester 104®, and Anise oil.

Another embodiment comprises cyclosporin A, Kemester 205®, and Anise oil.

Another embodiment comprises cyclosporin A, Kemester 226®, and Anise oil.

Another embodiment comprises cyclosporin A, Kemester 5221 SE®, and Anise oil.

Another embodiment comprises cyclosporin A, Kemester EGDS®, and Anise oil.

Another embodiment comprises cyclosporin A, Lexemul EGDS®, and Anise oil.

Another embodiment comprises cyclosporin A, Lexemul EGMS®, and Anise oil.

Another embodiment comprises cyclosporin A, Lexemul P®, and Anise oil.

Another embodiment comprises cyclosporin A, Lipo DGLS®, and Anise oil.

Another embodiment comprises cyclosporin A, Lipo EGDS®, and Anise.oil.

Another embodiment comprises cyclosporin A, Lipo PGMS®, and Anise oil.

Another embodiment comprises cyclosporin A, Liposorb S-4®, and Anise oil.

Another embodiment comprises cyclosporin A, Liposorb TO-20®, and Anise oil.

Another embodiment comprises cyclosporin A, Lumulse PGO®, and Anise oil.

Another embodiment comprises cyclosporin A, Mackester EGDS®, and Anise oil.

Another embodiment comprises cyclosporin A, Mackester EGMS®, and Anise oil.

Another embodiment comprises cyclosporin A, Mackester GSTP®, and Anise oil.

Another embodiment comprises cyclosporin A, Mackester Series®, and Anise oil.

Another embodiment comprises cyclosporin A, Magrabar PDG-50®, and Anise oil.

Another embodiment comprises cyclosporin A, Mapeg 6000 DS®, and Anise oil.

Another embodiment comprises cyclosporin A, Marlowet 4702®, and Anise oil.

Another embodiment comprises cyclosporin A, Monalube 305®, and Anise oil.

Another embodiment comprises cyclosporin A, Monalube 310®, and Anise oil.

Another embodiment comprises cyclosporin A, Monalube 315®, and Anise oil.

Another embodiment comprises cyclosporin A, Monalube 320®, and Anise oil.

Another embodiment comprises cyclosporin A, Monalube 325®, and Anise oil.

Another embodiment comprises cyclosporin A, Monalube 330®, and Anise oil.

Another embodiment comprises cyclosporin A, Naturechem PGHS®, and Anise oil.

Another embodiment comprises cyclosporin A, Polycastorol PLO-840®, and Anise oil.

Another embodiment comprises cyclosporin A, Polytex 1 OM®, and Anise oil.

Another embodiment comprises cyclosporin A, Ritasynt IP®, and Anise oil.

Another embodiment comprises cyclosporin A, Ross Chem PEG 600 DT®, and Anise oil.

Another embodiment comprises cyclosporin A, Schercemol PGMS®, and Anise oil.

Another embodiment comprises cyclosporin A, Sponto H-44C®, and Anise oil.

Another embodiment comprises cyclosporin A, Tegin G®, and Anise oil.

Another embodiment comprises cyclosporin A, Witbreak DGE-182®, and Anise oil.

Another embodiment comprises cyclosporin A, Witbreak DGE-182®, and Anise oil.

Another embodiment comprises cyclosporin A, Witbreak DRA-21®, and Anise oil.

Another embodiment comprises cyclosporin A, Witbreak DRA-21®, and Anise oil.

Another embodiment comprises cyclosporin A, Witbreak DRA-50®, and Anise oil.

Another embodiment comprises cyclosporin A, Witbreak DRA-50®, and Anise oil.

Another embodiment comprises cyclosporin A, Witconol F26-46®, and Anise oil.

Another embodiment comprises cyclosporin A, Witconol H-32®, and Anise oil.

Another embodiment comprises cyclosporin A, Witconol H-33®, and Anise oil.

Another embodiment comprises cyclosporin A, Witconol H-35A®, and Anise oil.

Another embodiment comprises cyclosporin A, Witconol RHP®, and Anise oil.

Another embodiment comprises cyclosporin A, a Lanolin-based Derivative, and Anise oil.

Another embodiment comprises cyclosporin A, Amerchol CAB®, and Anise oil.

Another embodiment comprises cyclosporin A, Amerchol L-101®, and Anise oil.

Another embodiment comprises cyclosporin A, Amerlate LFA-LO®, and Anise oil.

Another embodiment comprises cyclosporin A, Amerlate P®, and Anise oil.

Another embodiment comprises cyclosporin A, Barre Common Degras®, and Anise oil.

Another embodiment comprises cyclosporin A, Cholesterol NF®, and Anise oil.

Another embodiment comprises cyclosporin A, Crodalan AWS®, and Anise oil.

Another embodiment comprises cyclosporin A, Crodalan LA®, and Anise oil.

Another embodiment comprises cyclosporin A, Emery 1650®, and Anise oil.

Another embodiment comprises cyclosporin A, Emery 1650®, and Anise oil.

Another embodiment comprises cyclosporin A, Emery 1740®, and Anise oil.

Another embodiment comprises cyclosporin A, Emery 1740®, and Anise oil.

Another embodiment comprises cyclosporin A, Forlan 500®, and Anise oil.

Another embodiment comprises cyclosporin A, Forlan L®, and Anise oil.

Another embodiment comprises cyclosporin A, Laneto 50®, and Anise oil.

Another embodiment comprises cyclosporin A, Laneto 100®, and Anise oil.

Another embodiment comprises cyclosporin A, Laneto AWS®, and Anise oil.

Another embodiment comprises cyclosporin A, Lanfrax 1776®, and Anise oil.

Another embodiment comprises cyclosporin A, Lanfrax 1776®, and Anise oil.

Another embodiment comprises cyclosporin A, Lanogel 21®, and Anise oil.

Another embodiment comprises cyclosporin A, Lipolan®, and Anise oil.

Another embodiment comprises cyclosporin A, Lipolan 31®, and Anise oil.

Another embodiment comprises cyclosporin A, OHlan®, and Anise oil.

Another embodiment comprises cyclosporin A, Polychol 5®, and Anise oil.

Another embodiment comprises cyclosporin A, Polychol 15®, and Anise oil.

Another embodiment comprises cyclosporin A, Ritacetyl®, and Anise oil.

Another embodiment comprises cyclosporin A, Ritachol®, and Anise oil.

Another embodiment comprises cyclosporin A, Ritahydrox®, and Anise oil.

Another embodiment comprises cyclosporin A, Ritalafa®, and Anise oil.

Another embodiment comprises cyclosporin A, Ritalan®, and Anise oil.

Another embodiment comprises cyclosporin A, Ritalan AWS®, and Anise oil.

Another embodiment comprises cyclosporin A, Ritalan C®, and Anise oil.

Another embodiment comprises cyclosporin A, Ritawax®, and Anise oil.

Another embodiment comprises cyclosporin A, Ritawax AEO®, and Anise oil.

Another embodiment comprises cyclosporin A, Ritawax ALA®, and Anise oil.

Another embodiment comprises cyclosporin A, Solan/Solan 50/Super Solan®, and Anise oil.

Another embodiment comprises cyclosporin A, Super Hartolan/Hartolan®, and Anise oil.

Another embodiment comprises cyclosporin A, Supersat AWS-4®, and Anise oil.

Another embodiment comprises cyclosporin A, Supersat AWS-24®, and Anise oil.

Another embodiment comprises cyclosporin A, a Lecithin or Lecithin Derivative, and Anise oil.

Another embodiment comprises cyclosporin A, Alcolec®, and Anise oil.

Another embodiment comprises cyclosporin A, Lecithin®, and Anise oil.

Another embodiment comprises cyclosporin A, Lexin K®, and Anise oil.

Another embodiment comprises cyclosporin A, Natipide®, and Anise oil.

Another embodiment comprises cyclosporin A, a Lignin or Lignin Derivative, and Anise oil.

Another embodiment comprises cyclosporin A, Diwatex XP 9®, and Anise oil.

Another embodiment comprises cyclosporin A, Dynasperse LCD®, and Anise oil.

Another embodiment comprises cyclosporin A, Indulin SAL®, and Anise oil.

Another embodiment comprises cyclosporin A, Indulin W-1®, and Anise oil.

Another embodiment comprises cyclosporin A, Indulin W-5®, and Anise oil.

Another embodiment comprises cyclosporin A, Lignosol FTA®, and Anise oil.

Another embodiment comprises cyclosporin A, Lignosol SFX-65®, and Anise oil.

Another embodiment comprises cyclosporin A, Marasperse 52 CP®, and Anise oil.

Another embodiment comprises cyclosporin A, Marasperse AG®, and Anise oil.

Another embodiment comprises cyclosporin A, Marasperse CBOS-4®, and Anise oil.

Another embodiment comprises cyclosporin A, Ufoxane 2®, and Anise oil.

Another embodiment comprises cyclosporin A, a Methyl Ester, and Anise oil.

Another embodiment comprises cyclosporin A, E.B. Cleaner AK®, and Anise oil.

Another embodiment comprises cyclosporin A, Oleocal ME-70®, and Anise oil.

Another embodiment comprises cyclosporin A, Trade Name Company®, and Anise oil.

Another embodiment comprises cyclosporin A, Methyl Esters (cont'd)®, and Anise oil.

Another embodiment comprises cyclosporin A, Oleocal ME-92®, and Anise oil.

Another embodiment comprises cyclosporin A, Oleocal ME-112®, and Anise oil.

Another embodiment comprises cyclosporin A, Oleocal ME-130®, and Anise oil.

Another embodiment comprises cyclosporin A, a Monoglyceride or a Derivative thereof, and Anise oil.

Another embodiment comprises cyclosporin A, Dynacet 211®, and Anise oil.

Another embodiment comprises cyclosporin A, Hetsorb S-200, and Anise oil.

Another embodiment comprises cyclosporin A, Imwitor 191®, and Anise oil.

Another embodiment comprises cyclosporin A, Imwitor 370®, and Anise oil.

Another embodiment comprises cyclosporin A, Imwitor 375®, and Anise oil.

Another embodiment comprises cyclosporin A, Imwitor 900®, and Anise oil.

Another embodiment comprises cyclosporin A, Imwitor 945®, and Anise oil.

Another embodiment comprises cyclosporin A, Imwitor 2020®, and Anise oil.

Another embodiment comprises cyclosporin A, Kemester 5500®, and Anise oil.

Another embodiment comprises cyclosporin A, Kemester 6000®, and Anise oil.

Another embodiment comprises cyclosporin A, Magrabar GMC®, and Anise oil.

Another embodiment comprises cyclosporin A, Magrabar GMO-CK®, and Anise oil.

Another embodiment comprises cyclosporin A, Magrabar GPC-10®, and Anise oil.

Another embodiment comprises cyclosporin A, Magrabar MDG-5050®, and Anise oil.

Another embodiment comprises cyclosporin A, Monalube 335®, and Anise oil.

Another embodiment comprises cyclosporin A, Monoglycerides Glyceryl Monestearate Archer Daniels Midland Company®, and Anise oil.

Another embodiment comprises cyclosporin A, Rita GMS®, and Anise oil.

Another embodiment comprises cyclosporin A, Ritamulse SCG®, and Anise oil.

Another embodiment comprises cyclosporin A, Softigen 701®, and Anise oil.

Another embodiment comprises cyclosporin A, Tally 100 Plus®, and Anise oil.

Another embodiment comprises cyclosporin A, a Polyethylene Glycol, and Anise oil.

Another embodiment comprises cyclosporin A, Emulgade PL 68/50®, and Anise oil.

Another embodiment comprises cyclosporin A, Lumulse PEG®, and Anise oil.

Another embodiment comprises cyclosporin A, Rhodasurf PEG-400®, and Anise oil.

Another embodiment comprises cyclosporin A, Rhodasurf PEG-600®, and Anise oil.

Another embodiment comprises cyclosporin A, Witconol PEG-400®, and Anise oil.

Another embodiment comprises cyclosporin A, a Polymeric Surfactant, and Anise oil.

Another embodiment comprises cyclosporin A, Acritamer PNC-EG®, and Anise oil.

Another embodiment comprises cyclosporin A, Ag-Rho DEP-775®, and Anise oil.

Another embodiment comprises cyclosporin A, APG 325N Glycoside®, and Anise oil.

Another embodiment comprises cyclosporin A, Aristoflex AVC®, and Anise oil.

Another embodiment comprises cyclosporin A, Aristoflex HMB®, and Anise oil.

Another embodiment comprises cyclosporin A, Burco NPS-225®, and Anise oil.

Another embodiment comprises cyclosporin A, Burco NPS-816®, and Anise oil.

Another embodiment comprises cyclosporin A, Chemccinate 5603®, and Anise oil.

Another embodiment comprises cyclosporin A, Cosmedia Guar C-261 N®, and Anise oil.

Another embodiment comprises cyclosporin A, Gantrez S-95®, and Anise oil.

Another embodiment comprises cyclosporin A, Glucopon 220 UP®, and Anise oil.

Another embodiment comprises cyclosporin A, Glucopon 225 DK®, and Anise oil.

Another embodiment comprises cyclosporin A, Glucopon 425 N®, and Anise oil.

Another embodiment comprises cyclosporin A, Glucopon 600 UP®, and Anise oil.

Another embodiment comprises cyclosporin A, Glucopon 625 UP®, and Anise oil.

Another embodiment comprises cyclosporin A, Pemulen 1621®, and Anise oil.

Another embodiment comprises cyclosporin A, Pemulen 1622®, and Anise oil.

Another embodiment comprises cyclosporin A, Pemulen TR-1®, and Anise oil.

Another embodiment comprises cyclosporin A, Pemulen TR-2®, and Anise oil.

Another embodiment comprises cyclosporin A, Plantacare 818®, and Anise oil.

Another embodiment comprises cyclosporin A, Plantapon LGC Sorb®, and Anise oil.

Another embodiment comprises cyclosporin A, Plantaren 1200N®, and Anise oil.

Another embodiment comprises cyclosporin A, Plantaren 2000N®, and Anise oil.

Another embodiment comprises cyclosporin A, Viscolam AT 64®, and Anise oil.

Another embodiment comprises cyclosporin A, Viscolam AT 64P®, and Anise oil.

Another embodiment comprises cyclosporin A, Viscolam AT 100®, and Anise oil.

Another embodiment comprises cyclosporin A, Viscolam MAC 7®, and Anise oil.

Another embodiment comprises cyclosporin A, Viscolam SMC 20®, and Anise oil.

Another embodiment comprises cyclosporin A, Witbreak RTC-323®, and Anise oil.

Another embodiment comprises cyclosporin A, WSI 3700®, and Anise oil.

Another embodiment comprises cyclosporin A, a Propoxylated or Ethoxylated Fatty Acid, Alcohol, or Alkyl Phenol, and Anise oil.

Another embodiment comprises cyclosporin A, Antarox M-60®, and Anise oil.

Another embodiment comprises cyclosporin A, Antarox LF-224®, and Anise oil.

Another embodiment comprises cyclosporin A, Burcomul DFE-45®, and Anise oil.

Another embodiment comprises cyclosporin A, Burcoterge LFE-1000®, and Anise oil.

Another embodiment comprises cyclosporin A, Chemal LF-25B®, and Anise oil.

Another embodiment comprises cyclosporin A, Chemal LF-40B®, and Anise oil.

Another embodiment comprises cyclosporin A, Dehypon LS-36®, and Anise oil.

Another embodiment comprises cyclosporin A, Dehypon LS-36®, and Anise oil.

Another embodiment comprises cyclosporin A, Dehypon LS-54®, and Anise oil.

Another embodiment comprises cyclosporin A, Dehypon LS-54®, and Anise oil.

Another embodiment comprises cyclosporin A, Delonic 100 VLF®, and Anise oil.

Another embodiment comprises cyclosporin A, Delonic LF-60 MOD®, and Anise oil.

Another embodiment comprises cyclosporin A, Empiderm B®, and Anise oil.

Another embodiment comprises cyclosporin A, Ethylan 1206®, and Anise oil.

Another embodiment comprises cyclosporin A, Ethylan NS-500K®, and Anise oil.

Another embodiment comprises cyclosporin A, Ethylan NS-500LQ®, and Anise oil.

Another embodiment comprises cyclosporin A, Genapol 1392®, and Anise oil.

Another embodiment comprises cyclosporin A, Genapol 2317®, and Anise oil.

Another embodiment comprises cyclosporin A, Genapol 26EP710®, and Anise oil.

Another embodiment comprises cyclosporin A, Genapol EP 1022®, and Anise oil.

Another embodiment comprises cyclosporin A, Genapol EP 1024®, and Anise oil.

Another embodiment comprises cyclosporin A, Genapol EP 6068®, and Anise oil.

Another embodiment comprises cyclosporin A, Genapol NP915®, and Anise oil.

Another embodiment comprises cyclosporin A, Kieralon MFB®, and Anise oil.

Another embodiment comprises cyclosporin A, Lumisolve CSA-80 V®, and Anise oil.

Another embodiment comprises cyclosporin A, Marlowet 5001®, and Anise oil.

Another embodiment comprises cyclosporin A, Marlox FK 64®, and Anise oil.

Another embodiment comprises cyclosporin A, Marlox MO 124®, and Anise oil.

Another embodiment comprises cyclosporin A, Marlox S 58®, and Anise oil.

Another embodiment comprises cyclosporin A, Nonatell 1003®, and Anise oil.

Another embodiment comprises cyclosporin A, Nonatell 1038®, and Anise oil.

Another embodiment comprises cyclosporin A, Nonatell 1052®, and Anise oil.

Another embodiment comprises cyclosporin A, Nonatell 1061®, and Anise oil.

Another embodiment comprises cyclosporin Al Nonatell 1075®, and Anise oil.

Another embodiment comprises cyclosporin A, Nonatell 1088®, and Anise oil.

Another embodiment comprises cyclosporin A, Nonatell 1123®, and Anise oil.

Another embodiment comprises cyclosporin A, Nonatell 1153®, and Anise oil.

Another embodiment comprises cyclosporin A, Nonatell 1161®, and Anise oil.

Another embodiment comprises cyclosporin A, Nonatell 1172®, and Anise oil.

Another embodiment comprises cyclosporin A, Nonatell 1181®, and Anise oil.

Another embodiment comprises cyclosporin A, Norfox 36®, and Anise oil.

Another embodiment comprises cyclosporin A, Procetyl AWS®, and Anise oil.

Another embodiment comprises cyclosporin A, Sandoxylate SX 412®, and Anise oil.

Another embodiment comprises cyclosporin A, Sandoxylate SX 418®, and Anise oil.

Another embodiment comprises cyclosporin A, Surfonic JL-80X®, and Anise oil.

Another embodiment comprises cyclosporin A, Surfonic JL-80X-B1®, and Anise oil.

Another embodiment comprises cyclosporin A, Surfonic L4-29X®, and Anise oil.

Another embodiment comprises cyclosporin A, Suronic LF®, and Anise oil.

Another embodiment comprises cyclosporin A, T-Det A826®, and Anise oil.

Another embodiment comprises cyclosporin A, T-Det LF-416®, and Anise oil.

Another embodiment comprises cyclosporin A, Tergitol Min-Foam 1X®, and Anise oil.

Another embodiment comprises cyclosporin A, Tergitol Min-Foam 2X®, and Anise oil.

Another embodiment comprises cyclosporin A, Triton CF-21®, and Anise oil.

Another embodiment comprises cyclosporin A, Triton CF-76®, and Anise oil.

Another embodiment comprises cyclosporin A, Triton XL-80N®, and Anise oil.

Another embodiment comprises cyclosporin A, Witconol NS-98®, and Anise oil.

Another embodiment comprises cyclosporin A, Witconol NS-108LQ®, and Anise oil.

Another embodiment comprises cyclosporin A, Witconol NS-145®, and Anise oil.

Another embodiment comprises cyclosporin A, Witconol NS-179®, and Anise oil.

Another embodiment comprises cyclosporin A, a Protein-based Surfactant, and Anise oil.

Another embodiment comprises cyclosporin A, AminoFoam W®, and Anise oil.

Another embodiment comprises cyclosporin A, Amiter LGOD-2®, and Anise oil.

Another embodiment comprises cyclosporin A, Amiter LGS-2®, and Anise oil.

Another embodiment comprises cyclosporin A, Amiter LGS-5®, and Anise oil.

Another embodiment comprises cyclosporin A, Lamepon S®, and Anise oil.

Another embodiment comprises cyclosporin A, Lamepon S®, and Anise oil.

Another embodiment comprises cyclosporin A, Maypon 4C®, and Anise oil.

Another embodiment comprises cyclosporin A, May-Tein C®, and Anise oil.

Another embodiment comprises cyclosporin A, May-Tein CT®, and Anise oil.

Another embodiment comprises cyclosporin A, May-Tein KTS®, and Anise oil.

Another embodiment comprises cyclosporin A, May-Tein SY®, and Anise oil.

Another embodiment comprises cyclosporin A, Plantapon S®, and Anise oil.

Another embodiment comprises cyclosporin A, Proteol APL®, and Anise oil.

Another embodiment comprises cyclosporin A, Proteol OAT®, and Anise oil.

Another embodiment comprises cyclosporin A, Pyroter CPI-40®, and Anise oil.

Another embodiment comprises cyclosporin A, Pyroter GPI-25®, and Anise oil.

Another embodiment comprises cyclosporin A, Supro-Tein S®, and Anise oil.

Another embodiment comprises cyclosporin A, Supro-Tein V®, and Anise oil.

Another embodiment comprises cyclosporin A,® and Anise oil.

Another embodiment comprises cyclosporin A, a Sarcosine Derivative®, and Anise oil.

Another embodiment comprises cyclosporin A, Crodasinic LS-30®, and Anise oil.

Another embodiment comprises cyclosporin A, Vanseal CS®, and Anise oil.

Another embodiment comprises cyclosporin A, Vanseal LS®, and Anise oil.

Another embodiment comprises cyclosporin A, Vanseal MS®, and Anise oil.

Another embodiment comprises cyclosporin A, Vanseal NACS-30®, and Anise oil.

Another embodiment comprises cyclosporin A, Vanseal NALS-95®, and Anise oil.

Another embodiment comprises cyclosporin A, Vanseal OS®, and Anise oil.

Another embodiment comprises cyclosporin A,® and Anise oil.

Another embodiment comprises cyclosporin A, a Silicone-based Suractant®, and Anise oil.

Another embodiment comprises cyclosporin A, Abil-B-9950®, and Anise oil.

Another embodiment comprises cyclosporin A, Abil Care 85®, and Anise oil.

Another embodiment comprises cyclosporin A, Abil EM 90®, and Anise oil.

Another embodiment comprises cyclosporin A, Abil EM 97®, and Anise oil.

Another embodiment comprises cyclosporin A, Abil WE-09®, and Anise oil.

Another embodiment comprises cyclosporin A, Dow Corning 1248 Fluid®, and Anise oil.

Another embodiment comprises cyclosporin A, Dow Corning 3225C®, and Anise oil.

Another embodiment comprises cyclosporin A, Dow Corning 5200®, and Anise oil.

Another embodiment comprises cyclosporin A, Dow Corning 04-3667®, and Anise oil.

Another embodiment comprises cyclosporin A, Monasil PCA®, and Anise oil.

Another embodiment comprises cyclosporin A, Monasil PDM®, and Anise oil.

Another embodiment comprises cyclosporin A, Monasil PLN®, and Anise oil.

Another embodiment comprises cyclosporin A, Polyderm PPI-SI-WS®, and Anise oil.

Another embodiment comprises cyclosporin A, Troysol 380W®, and Anise oil.

Another embodiment comprises cyclosporin A, Troysol S366®, and Anise oil. Another embodiment comprises cyclosporin A, a Sorbitan Derivative, and Anise oil.

Another embodiment comprises cyclosporin A, Alkamuls SML®, and Anise oil.

Another embodiment comprises cyclosporin A, Alkamuls SMO®, and Anise oil.

Another embodiment comprises cyclosporin A, Alkamuls ST®, and Anise oil.

Another embodiment comprises cyclosporin A, Arlacel 20®, and Anise oil.

Another embodiment comprises cyclosporin A, Arlacel 40®, and Anise oil.

Another embodiment comprises cyclosporin A, Arlacel 60®, and Anise oil.

Another embodiment comprises cyclosporin A, Arlacel 80®, and Anise oil.

Another embodiment comprises cyclosporin A, Arlacel C®, and Anise oil.

Another embodiment comprises cyclosporin A, Armul 21®, and Anise oil.

Another embodiment comprises cyclosporin A, Atlox 80®, and Anise oil.

Another embodiment comprises cyclosporin A, Atlox 847®, and Anise oil.

Another embodiment comprises cyclosporin A, Atlox 1045A®, and Anise oil.

Another embodiment comprises cyclosporin A, Canarcel 20®, and Anise oil.

Another embodiment comprises cyclosporin A, Canarcel 60®, and Anise oil.

Another embodiment comprises cyclosporin A, Canarcel 80®, and Anise oil.

Another embodiment comprises cyclosporin A, Canarcel TW 20®, and Anise oil.

Another embodiment comprises cyclosporin A, Canarcel TW 60®, and Anise oil.

Another embodiment comprises cyclosporin A, Canarcel TW 80®, and Anise oil.

Another embodiment comprises cyclosporin A, Coladet BSB-P®, and Anise oil.

Another embodiment comprises cyclosporin A, Customulse O-20®, and Anise oil.

Another embodiment comprises cyclosporin A, Dehymuls E®, and Anise oil.

Another embodiment comprises cyclosporin A, DeSotan SMO®, and Anise oil.

Another embodiment comprises cyclosporin A, DeSotan SMO-20®, and Anise oil.

Another embodiment comprises cyclosporin A, DeSotan SMT®, and Anise oil.

Another embodiment comprises cyclosporin A, DeSotan SMT-20®, and Anise oil.

Another embodiment comprises cyclosporin A, Durfax 60®, and Anise oil.

Another embodiment comprises cyclosporin A, Durfax 65®, and Anise oil.

Another embodiment comprises cyclosporin A, Durfax 80®, and Anise oil.

Another embodiment comprises cyclosporin A, Durtan 60®, and Anise oil.

Another embodiment comprises cyclosporin A, Durtan 65®, and Anise oil.

Another embodiment comprises cyclosporin A, Liposorb L®, and Anise oil.

Another embodiment comprises cyclosporin A, Liposorb L-10®, and Anise oil.

Another embodiment comprises cyclosporin A, Liposorb L-20®, and Anise oil.

Another embodiment comprises cyclosporin A, Liposorb O®, and Anise oil.

Another embodiment comprises cyclosporin A, Liposorb O-20®, and Anise oil.

Another embodiment comprises cyclosporin A, Liposorb P®, and Anise oil.

Another embodiment comprises cyclosporin A, Liposorb P-20®, and Anise oil.

Another embodiment comprises cyclosporin A, Liposorb S®, and Anise oil.

Another embodiment comprises cyclosporin A, Liposorb S-20®, and Anise oil.

Another embodiment comprises cyclosporin A, Liposorb SQO®, and Anise oil.

Another embodiment comprises cyclosporin A, Liposorb TO®, and Anise oil.

Another embodiment comprises cyclosporin A, Liposorb TS®, and Anise oil.

Another embodiment comprises cyclosporin A, Liposorb TS-20®, and Anise oil.

Another embodiment comprises cyclosporin A, Lumisorb PS®, and Anise oil.

Another embodiment comprises cyclosporin A, Lumisorb SMO (T)®, and Anise oil.

Another embodiment comprises cyclosporin A, Lumisorb SMS K®, and Anise oil.

Another embodiment comprises cyclosporin A, Lumisorb SSO®, and Anise oil.

Another embodiment comprises cyclosporin A, Lumisorb STS K®, and Anise oil.

Another embodiment comprises cyclosporin A, Lumisorb STT®, and Anise oil.

Another embodiment comprises cyclosporin A, Magrabar SMO®, and Anise oil.

Another embodiment comprises cyclosporin A, Magrabar SMO-VEG®, and Anise oil.

Another embodiment comprises cyclosporin A, Magrabar SMT®, and Anise oil.

Another embodiment comprises cyclosporin A, Magrabar STO®, and Anise oil.

Another embodiment comprises cyclosporin A, Miracare BC-27®, and Anise oil.

Another embodiment comprises cyclosporin A, Ritabate 20®, and Anise oil.

Another embodiment comprises cyclosporin A, Ritabate 40®, and Anise oil.

Another embodiment comprises cyclosporin A, Ritabate 60®, and Anise oil.

Another embodiment comprises cyclosporin A, Ritabate 80®, and Anise oil.

Another embodiment comprises cyclosporin A, T-Maz®, and Anise oil.

Another embodiment comprises cyclosporin A, Tego SML®, and Anise oil.

Another embodiment comprises cyclosporin A, Tego SML 20®, and Anise oil.

Another embodiment comprises cyclosporin A, Tego SMO 80 V®, and Anise oil.

Another embodiment comprises cyclosporin A, Tego SMO V®, and Anise oil.

Another embodiment comprises cyclosporin A, Tego SMS®, and Anise oil.

Another embodiment comprises cyclosporin A, Tego STO V®, and Anise oil.

Another embodiment comprises cyclosporin A, Tween 21®, and Anise oil.

Another embodiment comprises cyclosporin A, Tween 40®, and Anise oil.

Another embodiment comprises cyclosporin A, Tween 60®, and Anise oil.

Another embodiment comprises cyclosporin A, Tween 60 K®, and Anise oil.

Another embodiment comprises cyclosporin A, Tween 61®, and Anise oil.

Another embodiment comprises cyclosporin A, Tween 65®, and Anise oil.

Another embodiment comprises cyclosporin A, Tween 80®, and Anise oil.

Another embodiment comprises cyclosporin A, Tween 80 K®, and Anise oil.

Another embodiment comprises cyclosporin A, Tween 81®, and Anise oil.

Another embodiment comprises cyclosporin A, Tween 85®, and Anise oil.

Another embodiment comprises cyclosporin A, a Sucrose or Glucose Ester, or Derivative thereof, and Anise oil.

Another embodiment comprises cyclosporin A, DeSulf GOS-P-60WCG®, and Anise oil.

Another embodiment comprises cyclosporin A, Glucam E-20 Distearate®, and Anise oil.

Another embodiment comprises cyclosporin A, Glucamate DOE-120®, and Anise oil.

Another embodiment comprises cyclosporin A, Glucamate SSE-20®, and Anise oil.

Another embodiment comprises cyclosporin A, Glucate DO®, and Anise oil.

Another embodiment comprises cyclosporin A, Glucate SS®, and Anise oil.

Another embodiment comprises cyclosporin A, Glucopon 425 UP®, and Anise oil.

Another embodiment comprises cyclosporin A, Isolan IS®, and Anise oil.

Another embodiment comprises cyclosporin A, Mazon 40®, and Anise oil.

Another embodiment comprises cyclosporin A, Montanov 82®, and Anise oil.

Another embodiment comprises cyclosporin A, Montanov 202®, and Anise oil.

Another embodiment comprises cyclosporin A, Montanov S®, and Anise oil.

Another embodiment comprises cyclosporin A, Rheozan®, and Anise oil.

Another embodiment comprises cyclosporin A, Simulsol AS 48®, and Anise oil.

Another embodiment comprises cyclosporin A, Simulsol SL 4®, and Anise oil.

Another embodiment comprises cyclosporin A, Simulsol SL 10®, and Anise oil.

Another embodiment comprises cyclosporin A, Simulsol SL 11 W®, and Anise oil.

Another embodiment comprises cyclosporin A, Simulsol SL 55®, and Anise oil.

Another embodiment comprises cyclosporin A, Suga Nate 100 and 160®, and Anise oil.

Another embodiment comprises cyclosporin A, Tego Care 450®, and Anise oil.

Another embodiment comprises cyclosporin A, Tego Care CG 90®, and Anise oil.

Another embodiment comprises cyclosporin A, Tego Care PS®, and Anise oil.

Another embodiment comprises cyclosporin A, Tegosoft PSE 141 G®, and Anise oil.

Another embodiment comprises cyclosporin A, Tegotens G 826®, and Anise oil.

Another embodiment comprises cyclosporin A, Triton BG-10 (70%)®, and Anise oil.

Another embodiment comprises cyclosporin A, Triton CG-110 (60%)®, and Anise oil.

Another embodiment comprises cyclosporin A, Wickenol 545®, and Anise oil.

Another embodiment comprises cyclosporin A, an alcohol, and Castor oil.

Another embodiment comprises cyclosporin A, Diglycerol®, and Castor oil.

Another embodiment comprises cyclosporin A, Hetoxide GT-80®, and Castor oil.

Another embodiment comprises cyclosporin A, Lexemul BEO®, and Castor oil.

Another embodiment comprises cyclosporin A, Polyglycerol-3®, and Castor oil.

Another embodiment comprises cyclosporin A, Redicote E Series®, and Castor oil.

Another embodiment comprises cyclosporin A, Simulsol OX 1005L®, and Castor oil.

Another embodiment comprises cyclosporin A, Stanfax 567®, and Castor oil.

Another embodiment comprises cyclosporin A, TA-1618®, and Castor oil.

Another embodiment comprises cyclosporin A, Witconol H-31A®, and Castor oil.

Another embodiment comprises cyclosporin A, an amine oxide, and Castor oil.

Another embodiment comprises cyclosporin A, AO-405®, and Castor oil.

Another embodiment comprises cyclosporin A, AO-455®, and Castor oil.

Another embodiment comprises cyclosporin A, AO 728 Special®, and Castor oil.

Another embodiment comprises cyclosporin A, Barlox 12®, and Castor oil.

Another embodiment comprises cyclosporin A, Barlox 14®, and Castor oil.

Another embodiment comprises cyclosporin A, Burcoxide Lo®, and Castor oil.

Another embodiment comprises cyclosporin A, Caloxamine LO®, and Castor oil.

Another embodiment comprises cyclosporin A, Chemoxide CAW®, and Castor oil.

Another embodiment comprises cyclosporin A, Chemoxide LM-30®, and Castor oil.

Another embodiment comprises cyclosporin A, Chemoxide LO®, and Castor oil.

Another embodiment comprises cyclosporin A, Chemoxide MO®, and Castor oil.

Another embodiment comprises cyclosporin A, Colalux CAO-35®, and Castor oil.

Another embodiment comprises cyclosporin A, Colalux LO®, and Castor oil.

Another embodiment comprises cyclosporin A, DeMox CAPO®, and Castor oil.

Another embodiment comprises cyclosporin A, DeMox CSG-30®, and Castor oil.

Another embodiment comprises cyclosporin A, DeMox LAO®, and Castor oil.

Another embodiment comprises cyclosporin A, Emcol L®, and Castor oil.

Another embodiment comprises cyclosporin A, Empigen OB®, and Castor oil.

Another embodiment comprises cyclosporin A, Empigen OS/A®, and Castor oil.

Another embodiment comprises cyclosporin A, Foamox CDO®, and Castor oil.

Another embodiment comprises cyclosporin A, Foamox DMM®, and Castor oil.

Another embodiment comprises cyclosporin A, Foamox DMS®, and Castor oil.

Another embodiment comprises cyclosporin A, Genaminox KC®, and Castor oil.

Another embodiment comprises cyclosporin A, Genaminox LA®, and Castor oil.

Another embodiment comprises cyclosporin A, Hartofoam SAO®, and Castor oil.

Another embodiment comprises cyclosporin A, Hartox DMCD®, and Castor oil.

Another embodiment comprises cyclosporin A, Lipowax DAT®, and Castor oil.

Another embodiment comprises cyclosporin A, Lipowax PB Pastilles®, and Castor oil.

Another embodiment comprises cyclosporin A, Mackamine C8®, and Castor oil.

Another embodiment comprises cyclosporin A, Mackamine C10®, and Castor oil.

Another embodiment comprises cyclosporin A, Mackamine C14®, and Castor oil.

Another embodiment comprises cyclosporin A, Mackamine CAO®, and Castor oil.

Another embodiment comprises cyclosporin A, Mackamine CO®, and Castor oil.

Another embodiment comprises cyclosporin A, Mackamine LO®, and Castor oil.

Another embodiment comprises cyclosporin A, Mackamine O2®, and Castor oil.

Another embodiment comprises cyclosporin A, Mackamine SAO®, and Castor oil.

Another embodiment comprises cyclosporin A, Mackamine SO®, and Castor oil.

Another embodiment comprises cyclosporin A, Mazox KCAO®, and Castor oil.

Another embodiment comprises cyclosporin A, Monalac MO®, and Castor oil.

Another embodiment comprises cyclosporin A, Norfox LDA®, and Castor oil.

Another embodiment comprises cyclosporin A, Rhodamox LO®, and Castor oil.

Another embodiment comprises cyclosporin A, Schercamox C-AA®, and Castor oil.

Another embodiment comprises cyclosporin A, Schercamox DMA®, and Castor oil.

Another embodiment comprises cyclosporin A, Schercamox DML®, and Castor oil.

Another embodiment comprises cyclosporin A, Schercamox DMM®, and Castor oil.

Another embodiment comprises cyclosporin A, Schercamox DMS®, and Castor oil.

Another embodiment comprises cyclosporin A, Tegotens DO®, and Castor oil.

Another embodiment comprises cyclosporin A, Tomah AO-14-2®, and Castor oil.

Another embodiment comprises cyclosporin A, Triaminox CDO®, and Castor oil.

Another embodiment comprises cyclosporin A, a block polymer, and Castor oil.

Another embodiment comprises cyclosporin A, AL 2070®, and Castor oil.

Another embodiment comprises cyclosporin A, Antarox 1 7-R-2®, and Castor oil.

Another embodiment comprises cyclosporin A, Antarox 25-R-2®, and Castor oil.

Another embodiment comprises cyclosporin A, Antarox 31-R-1®, and Castor oil.

Another embodiment comprises cyclosporin A, Antarox P-84®, and Castor oil.

Another embodiment comprises cyclosporin A, Antarox P-104/H®, and Castor oil.

Another embodiment comprises cyclosporin A, Arnox BP-Series®, and Castor oil.

Another embodiment comprises cyclosporin A, Chemonic 435®, and Castor oil.

Another embodiment comprises cyclosporin A, Chemonic D-25®, and Castor oil.

Another embodiment comprises cyclosporin A, Chemonic PL Series®, and Castor oil.

Another embodiment comprises cyclosporin A, Ethox L-121®, and Castor oil.

Another embodiment comprises cyclosporin A, Ethox L-122®, and Castor oil.

Another embodiment comprises cyclosporin A, Genapol PF-10®, and Castor oil.

Another embodiment comprises cyclosporin A, Genapol PF-20®, and Castor oil.

Another embodiment comprises cyclosporin A, Genapol PF-40A®, and Castor oil.

Another embodiment comprises cyclosporin A, Norfox 2-LF®, and Castor oil.

Another embodiment comprises cyclosporin A, Simulsol NW 342®, and Castor oil.

Another embodiment comprises cyclosporin A, T-Det BP-1®, and Castor oil.

Another embodiment comprises cyclosporin A, T-Det XD®, and Castor oil.

Another embodiment comprises cyclosporin A, T-Det XH®, and Castor oil.

Another embodiment comprises cyclosporin A, Triton CF-32®, and Castor oil.

Another embodiment comprises cyclosporin A, Witconol 171®, and Castor oil.

Another embodiment comprises cyclosporin A, Witconol 324®, and Castor oil.

Another embodiment comprises cyclosporin A, Witconol 324D®, and Castor oil.

Another embodiment comprises cyclosporin A, Witconol PD-2000®, and Castor oil.

a Carboxylated Alcohol or Alkylphenol Ethoxylate, and Castor oil.

Another embodiment comprises cyclosporin A, Emcol CN-6®, and Castor oil.

Another embodiment comprises cyclosporin A, Ethcarb®, and Castor oil.

Another embodiment comprises cyclosporin A, Gemtex WNT-Conc®, and Castor oil.

Another embodiment comprises cyclosporin A, Incrodet TD7-C®, and Castor oil.

Another embodiment comprises cyclosporin A, Marlinat CM 105/80®, and Castor oil.

Another embodiment comprises cyclosporin A, Marlowet 1072®, and Castor oil.

Another embodiment comprises cyclosporin A, Marlowet 4530®, and Castor oil.

Another embodiment comprises cyclosporin A, Marlowet 4530 LF®, and Castor oil.

Another embodiment comprises cyclosporin A, Marlowet 4534®, and Castor oil.

Another embodiment comprises cyclosporin A, Marlowet 4538®, and Castor oil.

Another embodiment comprises cyclosporin A, Marlowet 4539®, and Castor oil.

Another embodiment comprises cyclosporin A, Marlowet 4539 LF®, and Castor oil.

Another embodiment comprises cyclosporin A, Marlowet 4541®, and Castor oil.

Another embodiment comprises cyclosporin A, Miranate LEC-80®, and Castor oil.

Another embodiment comprises cyclosporin A, Sandopan B®, and Castor oil.

Another embodiment comprises cyclosporin A, Sandopan B Modified®, and Castor oil.

Another embodiment comprises cyclosporin A, Sandopan LS-24 Gel®, and Castor oil.

Another embodiment comprises cyclosporin A, Surfine T-A®, and Castor oil.

Another embodiment comprises cyclosporin A, a Carboxylic Acid/Fatty Acid, and Castor oil.

Another embodiment comprises cyclosporin A, Colaterge RAM®, and Castor oil.

Another embodiment comprises cyclosporin A, Colatrope INC®, and Castor oil.

Another embodiment comprises cyclosporin A, Crodacid B®, and Castor oil.

Another embodiment comprises cyclosporin A, DeTrope CA-100®, and Castor oil.

Another embodiment comprises cyclosporin A, Latol MTO®, and Castor oil.

Another embodiment comprises cyclosporin A, Lumulse CC-33 K®, and Castor oil.

Another embodiment comprises cyclosporin A, Mulls 2218®, and Castor oil.

Another embodiment comprises cyclosporin A, OL-600®, and Castor oil.

Another embodiment comprises cyclosporin A, OL-800®, and Castor oil.

Another embodiment comprises cyclosporin A, R-910®, and Castor oil.

Another embodiment comprises cyclosporin A, S-210®, and Castor oil.

Another embodiment comprises cyclosporin A, Sandopan DTC Acid®, and Castor oil.

Another embodiment comprises cyclosporin A, Sandopan LS 24 N®, and Castor oil.

Another embodiment comprises cyclosporin A, Sandopan MA-18®, and Castor oil.

Another embodiment comprises cyclosporin A, an Ethoxylated Alcohol, and Castor oil.

Another embodiment comprises cyclosporin A, Adsee 799®, and Castor oil.

Another embodiment comprises cyclosporin A, Adsee 799®, and Castor oil.

Another embodiment comprises cyclosporin A, Alfonic 610-3.5®, and Castor oil.

Another embodiment comprises cyclosporin A, Alfonic 810-2®, and Castor oil.

Another embodiment comprises cyclosporin A, Alfonic 810-6®, and Castor oil.

Another embodiment comprises cyclosporin A, Alfonic 1012-3®, and Castor oil.

Another embodiment comprises cyclosporin A, Alfonic 1012-5®, and Castor oil.

Another embodiment comprises cyclosporin A, Alfonic 1216CO-1.5®, and Castor oil.

Another embodiment comprises cyclosporin A, Alfonic 1216CO-7®, and Castor oil.

Another embodiment comprises cyclosporin A, Alfonic 1412-3®, and Castor oil.

Another embodiment comprises cyclosporin A, Alfonic 1412-7®, and Castor oil.

Another embodiment comprises cyclosporin A, Arlasolve 200®, and Castor oil.

Another embodiment comprises cyclosporin A, Arlasolve 200 Liquid®, and Castor oil.

Another embodiment comprises cyclosporin A, Armix 180-C®, and Castor oil.

Another embodiment comprises cyclosporin A, Armix 183®, and Castor oil.

Another embodiment comprises cyclosporin A, Armul 2404®, and Castor oil.

Another embodiment comprises cyclosporin A, Armul 2404®, and Castor oil.

Another embodiment comprises cyclosporin A, Atlas EMJ-C®, and Castor oil.

Another embodiment comprises cyclosporin A, Atlas G-2109®, and Castor oil.

Another embodiment comprises cyclosporin A, Atlas G-3886®, and Castor oil.

Another embodiment comprises cyclosporin A, Atlas G-3890®, and Castor oil.

Another embodiment comprises cyclosporin A, Bio Soft E-200®, and Castor oil.

Another embodiment comprises cyclosporin A, Bio Soft E-300®, and Castor oil.

Another embodiment comprises cyclosporin A, Bio Soft E-400®, and Castor oil.

Another embodiment comprises cyclosporin A, Bio Soft EN 600®, and Castor oil.

Another embodiment comprises cyclosporin A, Bio Soft TD-400®, and Castor oil.

Another embodiment comprises cyclosporin A, Bio Soft TD-630®, and Castor oil.

Another embodiment comprises cyclosporin A, Brij 30®, and Castor oil.

Another embodiment comprises cyclosporin A, Brij 52®, and Castor oil.

Another embodiment comprises cyclosporin A, Brij 56®, and Castor oil.

Another embodiment comprises cyclosporin A, Brij 58®, and Castor oil.

Another embodiment comprises cyclosporin A, Brij 72®, and Castor oil.

Another embodiment comprises cyclosporin A, Brij 76®, and Castor oil.

Another embodiment comprises cyclosporin A, Brij 78®, and Castor oil.

Another embodiment comprises cyclosporin A, Brij 93®, and Castor oil.

Another embodiment comprises cyclosporin A, Brij 97®, and Castor oil.

Another embodiment comprises cyclosporin A, Brij 98®, and Castor oil.

Another embodiment comprises cyclosporin A, Brij 700®, and Castor oil.

Another embodiment comprises cyclosporin A, Brij 700 S®, and Castor oil.

Another embodiment comprises cyclosporin A, Brij 721®, and Castor oil.

Another embodiment comprises cyclosporin A, Brij 721 S®, and Castor oil.

Another embodiment comprises cyclosporin A, Burcoterge CDG®, and Castor oil.

Another embodiment comprises cyclosporin A, Canasol AT 600®, and Castor oil.

Another embodiment comprises cyclosporin A, Canasol AT 800®, and Castor oil.

Another embodiment comprises cyclosporin A, Canasol AT 1200®, and Castor oil.

Another embodiment comprises cyclosporin A, Canasol BJ 35®, and Castor. oil.

Another embodiment comprises cyclosporin A, Canasol BJ 36®, and Castor oil.

Another embodiment comprises cyclosporin A, Canasol BJ 52®, and Castor oil.

Another embodiment comprises cyclosporin A, Canasol BJ 58®, and Castor oil.

Another embodiment comprises cyclosporin A, Canasol BJ 72®, and Castor oil.

Another embodiment comprises cyclosporin A, Canasol BJ 78®, and Castor oil.

Another embodiment comprises cyclosporin A, Canasol BJ 98®, and Castor oil.

Another embodiment comprises cyclosporin A, Canasol BJ 307®, and Castor oil.

Another embodiment comprises cyclosporin A, Cerfak 1400®, and Castor oil.

Another embodiment comprises cyclosporin A, Cetomacrogol 1000 BP®, and Castor oil.

Another embodiment comprises cyclosporin A, Chemonic C-2®, and Castor oil.

Another embodiment comprises cyclosporin A, Chemonic C-10®, and Castor oil.

Another embodiment comprises cyclosporin A, Chemonic C-20®, and Castor oil.

Another embodiment comprises cyclosporin A, Chemonic CT-12®, and Castor oil.

Another embodiment comprises cyclosporin A, Chemonic CT-20®, and Castor oil.

Another embodiment comprises cyclosporin A, Chemonic CT-30®, and Castor oil.

Another embodiment comprises cyclosporin A, Chemonic CT-55®, and Castor oil.

Another embodiment comprises cyclosporin A, Chemonic G-7®, and Castor oil.

Another embodiment comprises cyclosporin A, Chemonic G-26®, and Castor oil.

Another embodiment comprises cyclosporin A, Chemonic L-4®, and Castor oil.

Another embodiment comprises cyclosporin A, Chemonic L-7®, and Castor oil.

Another embodiment comprises cyclosporin A, Chemonic L-12®, and Castor oil.

Another embodiment comprises cyclosporin A, Chemonic L-2®, and Castor oil.

Another embodiment comprises cyclosporin A, Chemonic O-2®, and Castor oil.

Another embodiment comprises cyclosporin A, Chemonic O-5®, and Castor oil.

Another embodiment comprises cyclosporin A, Chemonic O-10®, and Castor oil.

Another embodiment comprises cyclosporin A, Chemonic O-20®, and Castor oil.

Another embodiment comprises cyclosporin A, Chemonic S-2®, and Castor oil.

Another embodiment comprises cyclosporin A, Chemonic S-10®, and Castor oil.

Another embodiment comprises cyclosporin A, Chemonic S-20®, and Castor oil.

Another embodiment comprises cyclosporin A, Colamulse FE®, and Castor oil.

Another embodiment comprises cyclosporin A, Cremophor A 20®, and Castor oil.

Another embodiment comprises cyclosporin A, Cremophor SA 2®, and Castor oil.

Another embodiment comprises cyclosporin A, Dehydol 100®, and Castor oil.

Another embodiment comprises cyclosporin A, Dehydol O-4®, and Castor oil.

Another embodiment comprises cyclosporin A, Delonic C-18®, and Castor oil.

Another embodiment comprises cyclosporin A, DeSonic 6T®, and Castor oil.

Another embodiment comprises cyclosporin A, DeSonic 9D®, and Castor oil.

Another embodiment comprises cyclosporin A, DeSonic 9T®, and Castor oil.

Another embodiment comprises cyclosporin A, DeSonic 12D®, and Castor oil.

Another embodiment comprises cyclosporin A, DeSonic 12T®, and Castor oil.

Another embodiment comprises cyclosporin A, DeSonic 15T®, and Castor oil.

Another embodiment comprises cyclosporin A, DeSonic TDA-9®, and Castor oil.

Another embodiment comprises cyclosporin A, DeThox GLG-7®, and Castor oil.

Another embodiment comprises cyclosporin A, DeThox GLG-26®, and Castor oil.

Another embodiment comprises cyclosporin A, DeThox LA-4®, and Castor oil.

Another embodiment comprises cyclosporin A, DeThox LA-23®, and Castor oil.

Another embodiment comprises cyclosporin A, DeThox SA-80®, and Castor oil.

Another embodiment comprises cyclosporin A, Disponil 05®, and Castor oil.

Another embodiment comprises cyclosporin A, Eccoterge EO-41B®, and Castor oil.

Another embodiment comprises cyclosporin A, Empilan KA2.5/90®, and Castor oil.

Another embodiment comprises cyclosporin A, Empilan KA5/90®, and Castor oil.

Another embodiment comprises cyclosporin A, Empilan KM-20®, and Castor oil.

Another embodiment comprises cyclosporin A, Empilan KM-50®, and Castor oil.

Another embodiment comprises cyclosporin A, Empilan L-23®, and Castor oil.

Another embodiment comprises cyclosporin A, Ethylan 25-3®, and Castor oil.

Another embodiment comprises cyclosporin A, Ethylan 1204®, and Castor oil.

Another embodiment comprises cyclosporin A, Ethylan DA-4®, and Castor oil.

Another embodiment comprises cyclosporin A, Ethylan LA-230®, and Castor oil.

Another embodiment comprises cyclosporin A, Ethylan SN®, and Castor oil.

Another embodiment comprises cyclosporin A, Ethylan TD-60®, and Castor oil.

Another embodiment comprises cyclosporin A, Ethylan TD-100®, and Castor oil.

Another embodiment comprises cyclosporin A, Ethylan TD-1407®, and Castor oil.

Another embodiment comprises cyclosporin A, Eumulgin B1®, and Castor oil.

Another embodiment comprises cyclosporin A, Eumulgin B2®, and Castor oil.

Another embodiment comprises cyclosporin A, Eumulgin B3®, and Castor oil.

Another embodiment comprises cyclosporin A, Eumulgin O-10®, and Castor oil.

Another embodiment comprises cyclosporin A, Flo Mo 80/20®, and Castor oil.

Another embodiment comprises cyclosporin A, Flo Mo Low Foam®, and Castor oil.

Another embodiment comprises cyclosporin A, Forlan C-24®, and Castor oil.

Another embodiment comprises cyclosporin A, Genapol 1454®, and Castor oil.

Another embodiment comprises cyclosporin A, Genapol BA-020®, and Castor oil.

Another embodiment comprises cyclosporin A, Genapol BA-040®, and Castor oil.

Another embodiment comprises cyclosporin A, Genapol C-100®, and Castor oil.

Another embodiment comprises cyclosporin A, Genapol DA 060®, and Castor oil.

Another embodiment comprises cyclosporin A, Genapol HS 020®, and Castor oil.

Another embodiment comprises cyclosporin A, Genapol HS 200®, and Castor oil.

Another embodiment comprises cyclosporin A, Genapol ID-040®, and Castor oil.

Another embodiment comprises cyclosporin A, Genapol ID-060®, and Castor oil.

Another embodiment comprises cyclosporin A, Genapol ID-090®, and Castor oil.

Another embodiment comprises cyclosporin A, Genapol LA 010®, and Castor oil.

Another embodiment comprises cyclosporin A, Genapol LA 020®, and Castor oil.

Another embodiment comprises cyclosporin A, Genapol LA 030®, and Castor oil.

Another embodiment comprises cyclosporin A, Genapol LA 040®, and Castor oil.

Another embodiment comprises cyclosporin A, Genapol LA 050®, and Castor oil.

Another embodiment comprises cyclosporin A, Genapol LA 060®, and Castor oil.

Another embodiment comprises cyclosporin A, Genapol LA 070®, and Castor oil.

Another embodiment comprises cyclosporin A, Genapol LA 070S®, and Castor oil.

Another embodiment comprises cyclosporin A, Genapol LA 230®, and Castor oil.

Another embodiment comprises cyclosporin A, Genapol O 020®, and Castor oil.

Another embodiment comprises cyclosporin A, Genapol O 050®, and Castor oil.

Another embodiment comprises cyclosporin A, Genapol O 100®, and Castor oil.

Another embodiment comprises cyclosporin A, Genapol O 200®, and Castor oil.

Another embodiment comprises cyclosporin A, Genapol SA 030®, and Castor oil.

Another embodiment comprises cyclosporin A, Genapol SA 120®, and Castor oil.

Another embodiment comprises cyclosporin A, Genapol T-020®, and Castor oil.

Another embodiment comprises cyclosporin A, Genapol UD-030®, and Castor oil.

Another embodiment comprises cyclosporin A, Genapol UD-050®, and Castor oil.

Another embodiment comprises cyclosporin A, Genapol UD-070®, and Castor oil.

Another embodiment comprises cyclosporin A, Genapol UD-079®, and Castor oil.

Another embodiment comprises cyclosporin A, Genapol UD-080®, and Castor oil.

Another embodiment comprises cyclosporin A, Genapol UD-110®, and Castor oil.

Another embodiment comprises cyclosporin A, Genapol X 030®, and Castor oil.

Another embodiment comprises cyclosporin A, Genapol X 050®, and Castor oil.

Another embodiment comprises cyclosporin A, Genapol X 060®, and Castor oil.

Another embodiment comprises cyclosporin A, Genapol X 070®, and Castor oil.

Another embodiment comprises cyclosporin A, Genapol X 080®, and Castor oil.

Another embodiment comprises cyclosporin A, Genapol X 100®, and Castor oil.

Another embodiment comprises cyclosporin A, Genapol X 159®, and Castor oil.

Another embodiment comprises cyclosporin A, Generol 122 E5®, and Castor oil.

Another embodiment comprises cyclosporin A, Generol 122 E25®, and Castor oil.

Another embodiment comprises cyclosporin A, Hostacerin T-3®, and Castor oil.

Another embodiment comprises cyclosporin A, Iconol LF 110®, and Castor oil.

Another embodiment comprises cyclosporin A, Incropol CS-20®, and Castor oil.

Another embodiment comprises cyclosporin A, Lexemul CS-20®, and Castor oil.

Another embodiment comprises cyclosporin A, Liponic EG-1®, and Castor oil.

Another embodiment comprises cyclosporin A, Lipowax D®, and Castor oil.

Another embodiment comprises cyclosporin A, Lipowax G®, and Castor oil.

Another embodiment comprises cyclosporin A, Lipowax NI®, and Castor oil.

Another embodiment comprises cyclosporin A, Lipowax P®, and Castor oil.

Another embodiment comprises cyclosporin A, Lipowax P-31®, and Castor oil.

Another embodiment comprises cyclosporin A, Lipowax PR®, and Castor oil.

Another embodiment comprises cyclosporin A, Lumulse CS-20®, and Castor oil.

Another embodiment comprises cyclosporin A, Macol CSA-20®, and Castor oil.

Another embodiment comprises cyclosporin A, Marlox B 24/50®, and Castor oil.

Another embodiment comprises cyclosporin A, Mazawet 77®, and Castor oil.

Another embodiment comprises cyclosporin A, Norfox 1713®, and Castor oil.

Another embodiment comprises cyclosporin A, Norfox 2579®, and Castor oil.

Another embodiment comprises cyclosporin A, Norfox Lo Foam®, and Castor oil.

Another embodiment comprises cyclosporin A, Promulgen D®, and Castor oil.

Another embodiment comprises cyclosporin A, Promulgen G®, and Castor oil.

Another embodiment comprises cyclosporin A, Renex 30®, and Castor oil.

Another embodiment comprises cyclosporin A, Renex 36®, and Castor oil.

Another embodiment comprises cyclosporin A, Rhodasurf A 24®, and Castor oil.

Another embodiment comprises cyclosporin A, Rhodasurf ME-10®, and Castor oil.

Another embodiment comprises cyclosporin A, Rhodasurf BEH-25®, and Castor oil.

Another embodiment comprises cyclosporin A, Rhodasurf BEH-40®, and Castor oil.

Another embodiment comprises cyclosporin A, Rhodasurf DA 530®, and Castor oil.

Another embodiment comprises cyclosporin A, Rhodasurf DA 630®, and Castor oil.

Another embodiment comprises cyclosporin A, Rhodasurf DA 639®, and Castor oil.

Another embodiment comprises cyclosporin A, Rhodasurf LAN-23®, and Castor.oil.

Another embodiment comprises cyclosporin A, Rhodasurf ON-870®, and Castor oil.

Another embodiment comprises cyclosporin A, Rhodasurf ON-877®, and Castor oil.

Another embodiment comprises cyclosporin A, Rhodasurf TB-970 FLK®, and Castor oil.

Another embodiment comprises cyclosporin A, Ritacet-20®, and Castor oil.

Another embodiment comprises cyclosporin A, Ritachol 1000®, and Castor oil.

Another embodiment comprises cyclosporin A, Ritachol 2000®, and Castor oil.

Another embodiment comprises cyclosporin A, Ritachol 5000®, and Castor oil.

Another embodiment comprises cyclosporin A, Ritox 35®, and Castor oil.

Another embodiment comprises cyclosporin A, Surfonic DA-4®, and Castor oil.

Another embodiment comprises cyclosporin A, Surfonic DA-6®, and Castor oil.

Another embodiment comprises cyclosporin A, Surfonic L46-7®, and Castor oil.

Another embodiment comprises cyclosporin A, Surfonic POA®, and Castor oil.

Another embodiment comprises cyclosporin A, Synthrapol KB®, and Castor oil.

Another embodiment comprises cyclosporin A, Teginacid®, and Castor oil.

Another embodiment comprises cyclosporin A, Teginacid C®, and Castor oil.

Another embodiment comprises cyclosporin A, Tegotens EC 11®, and Castor oil.

Another embodiment comprises cyclosporin A, Tinegal NA®, and Castor oil.

Another embodiment comprises cyclosporin A, Tomadol 400®, and Castor oil.

Another embodiment comprises cyclosporin A, Tomadol 600®, and Castor oil.

Another embodiment comprises cyclosporin A, Tomadol 900®, and Castor oil.

Another embodiment comprises cyclosporin A, Uniperol O®, and Castor oil.

Another embodiment comprises cyclosporin A, Witconol SN Series®, and Castor oil.

Another embodiment comprises cyclosporin A, an Ethoxylated Alkylphenol, and Castor oil.

Another embodiment comprises cyclosporin A, Antarox LF-222®, and Castor oil.

Another embodiment comprises cyclosporin A, Atlox 775®, and Castor oil.

Another embodiment comprises cyclosporin A, Caloxylate N-9®, and Castor oil.

Another embodiment comprises cyclosporin A, Canasol NF-1000®, and Castor oil.

Another embodiment comprises cyclosporin A, Canasol NF-3000®, and Castor oil.

Another embodiment comprises cyclosporin A, Canasol NF-3070®, and Castor oil.

Another embodiment comprises cyclosporin A, Canasol OF 1670®, and Castor oil.

Another embodiment comprises cyclosporin A, Canasol OF 2570®, and Castor oil.

Another embodiment comprises cyclosporin A, Canasol OF 4070®, and Castor oil.

Another embodiment comprises cyclosporin A, Chemax DNP-8®, and Castor oil.

Another embodiment comprises cyclosporin A, Chemax DNP-18®, and Castor oil.

Another embodiment comprises cyclosporin A, Chemax DNP-150/50®, and Castor oil.

Another embodiment comprises cyclosporin A, DeSonic 1.5N®, and Castor oil.

Another embodiment comprises cyclosporin A, DeSonic 4N®, and Castor oil.

Another embodiment comprises cyclosporin A, DeSonic 5N®, and Castor oil.

Another embodiment comprises cyclosporin A, DeSonic 6D®, and Castor oil.

Another embodiment comprises cyclosporin A, DeSonic 6N®, and Castor oil.

Another embodiment comprises cyclosporin A, DeSonic 7N®, and Castor oil.

Another embodiment comprises cyclosporin A, DeSonic 9N®, and Castor oil.

Another embodiment comprises cyclosporin A, DeSonic 10D®, and Castor oil.

Another embodiment comprises cyclosporin A, DeSonic 11N®, and Castor oil.

Another embodiment comprises cyclosporin A, DeSonic 12N®, and Castor oil.

Another embodiment comprises cyclosporin A, DeSonic 15N®, and Castor oil.

Another embodiment comprises cyclosporin A, DeSonic 20N®, and Castor oil.

Another embodiment comprises cyclosporin A, Eccoscour RC®, and Castor oil.

Another embodiment comprises cyclosporin A, Eccoterge EO-100®, and Castor oil.

Another embodiment comprises cyclosporin A, Emulsifier 632/90%®, and Castor oil.

Another embodiment comprises cyclosporin A, Geronol AG-821®, and Castor oil.

Another embodiment comprises cyclosporin A, Gradonic N-95®, and Castor oil.

Another embodiment comprises cyclosporin A, Hetoxide NP-4®, and Castor oil.

Another embodiment comprises cyclosporin A, Hetoxide NP-30®, and Castor oil.

Another embodiment comprises cyclosporin A, Hostapal N-100®, and Castor oil.

Another embodiment comprises cyclosporin A, Hostapal N-110®, and Castor oil.

Another embodiment comprises cyclosporin A, Igepal CTA-639W®, and Castor oil.

Another embodiment comprises cyclosporin A, Igepal DAP-9®, and Castor oil.

Another embodiment comprises cyclosporin A, Igepal OD-410®, and Castor oil.

Another embodiment comprises cyclosporin A, Igepal SS-837®, and Castor oil.

Another embodiment comprises cyclosporin A, Lipocol NP-9 USP®, and Castor oil.

Another embodiment comprises cyclosporin A, Macol DNP-10®, and Castor oil.

Another embodiment comprises cyclosporin A, Marlophen NP 5®, and Castor oil.

Another embodiment comprises cyclosporin A, Marlophen P 1®, and Castor oil.

Another embodiment comprises cyclosporin A, Surfonic NB®, and Castor oil.

Another embodiment comprises cyclosporin A, Surfonic OPB-307®, and Castor oil.

Another embodiment comprises cyclosporin A, Surfonic OPB-407®, and Castor oil.

Another embodiment comprises cyclosporin A, Syn Fac 334®, and Castor oil.

Another embodiment comprises cyclosporin A, Syn Fac 8216®, and Castor oil.

Another embodiment comprises cyclosporin A, Triton N-57®, and Castor oil.

Another embodiment comprises cyclosporin A, Trycol 6956®, and Castor oil.

Another embodiment comprises cyclosporin A, Trycol 6961®, and Castor oil.

Another embodiment comprises cyclosporin A, Trycol 6964®, and Castor oil.

Another embodiment comprises cyclosporin A, Trycol 6969®, and Castor oil.

Another embodiment comprises cyclosporin A, Trycol 6974®, and Castor oil.

Another embodiment comprises cyclosporin A, Witbreak DRB-127®, and Castor oil.

Another embodiment comprises cyclosporin A, Witbreak DRB-127®, and Castor oil.

Another embodiment comprises cyclosporin A, Witconol NP Series®, and Castor oil.

Another embodiment comprises cyclosporin A, an Ethoxylated Aryl Phenol, and Castor oil.

Another embodiment comprises cyclosporin A, Soprophor BSU®, and Castor oil.

Another embodiment comprises cyclosporin A, Soprophor CY/8®, and Castor oil.

Another embodiment comprises cyclosporin A, Soprophor S/25®, and Castor oil.

Another embodiment comprises cyclosporin A, Witconol NIO®, and Castor oil.

Another embodiment comprises cyclosporin A, Witconol NIW®, and Castor oil.

Another embodiment comprises cyclosporin A, Witconol S-100®, and Castor oil.

Another embodiment comprises cyclosporin A, an Ethoxylated Fatty Acid, and Castor oil.

Another embodiment comprises cyclosporin A, Aldo PGHMS®, and Castor oil.

Another embodiment comprises cyclosporin A, Alkamuls TO-15/HR®, and Castor oil.

Another embodiment comprises cyclosporin A, Armotan AL-69-66®, and Castor oil.

Another embodiment comprises cyclosporin A, Cerasynt 840®, and Castor oil.

Another embodiment comprises cyclosporin A, Cerasynt 945®, and Castor oil.

Another embodiment comprises cyclosporin A, Crystal Inhibitor No. 5®, and Castor oil.

Another embodiment comprises cyclosporin A, DeThox Acid L-9®, and Castor oil.

Another embodiment comprises cyclosporin A, DeThox Acid S-8®, and Castor oil.

Another embodiment comprises cyclosporin A, Ethofat 242/25®, and Castor oil.

Another embodiment comprises cyclosporin A, Hydropalat 65®, and Castor oil.

Another embodiment comprises cyclosporin A, Lipo EGMS®, and Castor oil.

Another embodiment comprises cyclosporin A, Lipopeg 2 DL®, and Castor oil.

Another embodiment comprises cyclosporin A, Lipopeg 4 DL®, and Castor oil.

Another embodiment comprises cyclosporin A, Lipopeg 4-L®, and Castor oil.

Another embodiment comprises cyclosporin A, Lipopeg 39-S®, and Castor oil.

Another embodiment comprises cyclosporin A, Lipopeg 4-S®, and Castor oil.

Another embodiment comprises cyclosporin A, Lipopeg 10-S®, and Castor oil.

Another embodiment comprises cyclosporin A, Lipopeg 100-S®, and Castor oil.

Another embodiment comprises cyclosporin A, Lipopeg 6000 DS®, and Castor oil.

Another embodiment comprises cyclosporin A, Lumulse 40-L®, and Castor oil.

Another embodiment comprises cyclosporin A, Lumulse 40-S®, and Castor oil.

Another embodiment comprises cyclosporin A, Lumulse 42-L®, and Castor oil.

Another embodiment comprises cyclosporin A, Lumulse 42-S®, and Castor oil.

Another embodiment comprises cyclosporin A, Lumulse 100-S®, and Castor oil.

Another embodiment comprises cyclosporin A, Lumulse 602-S®, and Castor oil.

Another embodiment comprises cyclosporin A, Magrabar PGE-20-0®, and Castor oil.

Another embodiment comprises cyclosporin A, Magrabar PGE-20L®, and Castor oil.

Another embodiment comprises cyclosporin A, Magrabar PGE-20T®, and Castor oil.

Another embodiment comprises cyclosporin A, Magrabar PGE-22-0®, and Castor oil.

Another embodiment comprises cyclosporin A, Magrabar PGE-22L®, and Castor oil.

Another embodiment comprises cyclosporin A, Magrabar PGE-22T®, and Castor oil.

Another embodiment comprises cyclosporin A, Magrabar PGE-40-0®, and Castor oil.

Another embodiment comprises cyclosporin A, Magrabar PGE-40L®, and Castor oil.

Another embodiment comprises cyclosporin A, Magrabar PGE-40T®, and Castor oil.

Another embodiment comprises cyclosporin A, Magrabar PGE-42-0®, and Castor oil.

Another embodiment comprises cyclosporin A, Magrabar PGE-42L®, and Castor oil.

Another embodiment comprises cyclosporin A, Magrabar PGE-42T®, and Castor oil.

Another embodiment comprises cyclosporin A, Magrabar PGE-60-0®, and Castor oil.

Another embodiment comprises cyclosporin A, Magrabar PGE-60L®, and Castor oil.

Another embodiment comprises cyclosporin A, Magrabar PGE-60T®, and Castor oil.

Another embodiment comprises cyclosporin A, Magrabar PGE-62-0®, and Castor oil.

Another embodiment comprises cyclosporin A, Magrabar PGE-62L®, and Castor oil.

Another embodiment comprises cyclosporin A, Magrabar PGE-62T®, and Castor oil.

Another embodiment comprises cyclosporin A, Mapeg S-40K®, and Castor oil.

Another embodiment comprises cyclosporin A, Marlowet OTS®, and Castor oil.

Another embodiment comprises cyclosporin A, Naturechem PGR®, and Castor oil.

Another embodiment comprises cyclosporin A, PG No. 4®, and Castor oil.

Another embodiment comprises cyclosporin A, Renex 20®, and Castor oil.

Another embodiment comprises cyclosporin A, Ritox 52®, and Castor oil.

Another embodiment comprises cyclosporin A, Ritox 53®, and Castor oil.

Another embodiment comprises cyclosporin A, Ritox 59®, and Castor oil.

Another embodiment comprises cyclosporin A, Surfax 8916/A®, and Castor oil.

Another embodiment comprises cyclosporin A, Tego Acid S 40 P®, and Castor oil.

Another embodiment comprises cyclosporin A, Tego Acid S 100 P®, and Castor oil.

Another embodiment comprises cyclosporin A, Tween 20®, and Castor oil.

Another embodiment comprises cyclosporin A, Volpo 131®, and Castor oil.

Another embodiment comprises cyclosporin A, an Ethoxylated Fatty Ester or Oil, and Castor oil.

Another embodiment comprises cyclosporin A, Acconon 6-C10®, and Castor oil.

Another embodiment comprises cyclosporin A, Acconon CC-6®, and Castor oil.

Another embodiment comprises cyclosporin A, Acconon CO-7®, and Castor oil.

Another embodiment comprises cyclosporin A, Aldosperse 40/60 FG®, and Castor oil.

Another embodiment comprises cyclosporin A, Aldosperse ML-23®, and Castor oil.

Another embodiment comprises cyclosporin A, Aldosperse MS-20 FG®, and Castor oil.

Another embodiment comprises cyclosporin A, Alkamuls EL-620®, and Castor oil.

Another embodiment comprises cyclosporin A, Alkamuls EL-719®, and Castor oil.

Another embodiment comprises cyclosporin A, Alkamuls EL-985®, and Castor oil.

Another embodiment comprises cyclosporin A, Arlatone G®, and Castor oil.

Another embodiment comprises cyclosporin A, Arlatone T®, and Castor oil.

Another embodiment comprises cyclosporin A, Atlas G-1045A®, and Castor oil.

Another embodiment comprises cyclosporin A, Atlas G-1086®, and Castor oil.

Another embodiment comprises cyclosporin A, Atlas G-1087®, and Castor oil.

Another embodiment comprises cyclosporin A, Atlas G-1089®, and Castor oil.

Another embodiment comprises cyclosporin A, Atlas G-1096®, and Castor oil.

Another embodiment comprises cyclosporin A, Atlas G-1292®, and Castor oil.

Another embodiment comprises cyclosporin A, Atlas G-1293®, and Castor oil.

Another embodiment comprises cyclosporin A, Atlas G-1300®, and Castor oil.

Another embodiment comprises cyclosporin A, Atlas G-7076®, and Castor oil.

Another embodiment comprises cyclosporin A, Capmul EMG®, and Castor oil.

Another embodiment comprises cyclosporin A, Chemonic CO-40®, and Castor oil.

Another embodiment comprises cyclosporin A, Chemonic LI-3®, and Castor oil.

Another embodiment comprises cyclosporin A, Chemonic LI-7®, and Castor oil.

Another embodiment comprises cyclosporin A, Cirrasol GM®, and Castor oil.

Another embodiment comprises cyclosporin A, Cremophor CO 40®, and Castor oil.

Another embodiment comprises cyclosporin A, Cremophor CO 410®, and Castor oil.

Another embodiment comprises cyclosporin A, Cremophor EL®, and Castor oil.

Another embodiment comprises cyclosporin A, Cremophor GC7®, and Castor oil.

Another embodiment comprises cyclosporin A, Cremophor RH-40®, and Castor oil.

Another embodiment comprises cyclosporin A, Crovol A-40®, and Castor oil.

Another embodiment comprises cyclosporin A, Crovol A-70®, and Castor oil.

Another embodiment comprises cyclosporin A, Crovol M-70®, and Castor oil.

Another embodiment comprises cyclosporin A, Crovol PK-70®, and Castor oil.

Another embodiment comprises cyclosporin A, Cutina E-24®, and Castor oil.

Another embodiment comprises cyclosporin A, Dacospin 12-R®, and Castor oil.

Another embodiment comprises cyclosporin A, Dehymuls HRE-7®, and Castor oil.

Another embodiment comprises cyclosporin A, DeSonic 30C®, and Castor oil.

Another embodiment comprises cyclosporin A, DeSonic 36C®, and Castor oil.

Another embodiment comprises cyclosporin A, DeSonic 40C®, and Castor oil.

Another embodiment comprises cyclosporin A, Durfax 60®, and Castor oil.

Another embodiment comprises cyclosporin A, Durfax 65®, and Castor oil.

Another embodiment comprises cyclosporin A, Durfax 80®, and Castor oil.

Another embodiment comprises cyclosporin A, Durfax EOM®, and Castor oil.

Another embodiment comprises cyclosporin A, Eccoterge NF-2®, and Castor oil.

Another embodiment comprises cyclosporin A, Emulpon CO-360®, and Castor oil.

Another embodiment comprises cyclosporin A, Emulpon CO-550®, and Castor oil.

Another embodiment comprises cyclosporin A, Emulsogen EL®, and Castor oil.

Another embodiment comprises cyclosporin A, Emulsogen HCO 040®, and Castor oil.

Another embodiment comprises cyclosporin A, Emulsogen HCO 060®, and Castor oil.

Another embodiment comprises cyclosporin A, Emulsynt 1055®, and Castor oil.

Another embodiment comprises cyclosporin A, Ethox 3095®, and Castor oil.

Another embodiment comprises cyclosporin A, Eumulgin RO-40®, and Castor oil.

Another embodiment comprises cyclosporin A, Genapol G-260®, and Castor oil.

Another embodiment comprises cyclosporin A, Glycosperse L-20®, and Castor oil.

Another embodiment comprises cyclosporin A, Glycosperse O-5®, and Castor oil.

Another embodiment comprises cyclosporin A, Glycosperse O-20®, and Castor oil.

Another embodiment comprises cyclosporin A, Glycosperse O-20 FG®, and Castor oil.

Another embodiment comprises cyclosporin A, Glycosperse S-20®, and Castor oil.

Another embodiment comprises cyclosporin A, Glycosperse S-20 FG®, and Castor oil.

Another embodiment comprises cyclosporin A, Glycosperse TS-20®, and Castor oil.

Another embodiment comprises cyclosporin A, Glycosperse TS-20 FG®, and Castor oil.

Another embodiment comprises cyclosporin A, Hetan SL®, and Castor oil.

Another embodiment comprises cyclosporin A, Hetan SO®, and Castor oil.

Another embodiment comprises cyclosporin A, Hetan SS®, and Castor oil.

Another embodiment comprises cyclosporin A, Hetoxide C-2®, and Castor oil.

Another embodiment comprises cyclosporin A, Hetoxide C-9®, and Castor oil.

Another embodiment comprises cyclosporin A, Hetoxide C-15®, and Castor oil.

Another embodiment comprises cyclosporin A, Hetoxide C-25®, and Castor oil.

Another embodiment comprises cyclosporin A, Hetoxide C-40®, and Castor oil.

Another embodiment comprises cyclosporin A, Hetoxide C-200®, and Castor oil.

Another embodiment comprises cyclosporin A, Hetoxide C-200-50%®, and Castor oil.

Another embodiment comprises cyclosporin A, Hetoxide GC-30®, and Castor oil.

Another embodiment comprises cyclosporin A, Hetoxide HC-60®, and Castor oil.

Another embodiment comprises cyclosporin A, Ice No. 2®, and Castor oil.

Another embodiment comprises cyclosporin A, Incrocas 30/40®, and Castor oil.

Another embodiment comprises cyclosporin A, Lexol EC®, and Castor oil.

Another embodiment comprises cyclosporin A, Lexol EO®, and Castor oil.

Another embodiment comprises cyclosporin A, Lipocol HCO-40®, and Castor oil.

Another embodiment comprises cyclosporin A, Lipocol HCO-60®, and Castor oil.

Another embodiment comprises cyclosporin A, Lipocol O-3 Special®, and Castor oil.

Another embodiment comprises cyclosporin A, Lipopeg 2-L®, and Castor oil.

Another embodiment comprises cyclosporin A, Lipopeg 4-DO®, and Castor oil.

Another embodiment comprises cyclosporin A, Lipopeg 4-DS®, and Castor oil.

Another embodiment comprises cyclosporin A, Lipovol GTB®, and Castor oil.

Another embodiment comprises cyclosporin A, Lonzest SML-20®, and Castor oil.

Another embodiment comprises cyclosporin A, Lonzest SMO-20®, and Castor oil.

Another embodiment comprises cyclosporin A, Lonzest SMS-20®, and Castor oil.

Another embodiment comprises cyclosporin A, Lonzest STO-20®, and Castor oil.

Another embodiment comprises cyclosporin A, Lonzest STS-20®, and Castor oil.

Another embodiment comprises cyclosporin A, Lumulse GR-40®, and Castor oil.

Another embodiment comprises cyclosporin A, Lumulse GRH-40®, and Castor oil.

Another embodiment comprises cyclosporin A, Lumulse POE (7) GML®, and Castor oil.

Another embodiment comprises cyclosporin A, Lumulse POE (12) Glyc®, and Castor oil.

Another embodiment comprises cyclosporin A, Lumulse POE (40) MS KP®, and Castor oil.

Another embodiment comprises cyclosporin A, Marlowet 4750®, and Castor oil.

Another embodiment comprises cyclosporin A, Marlowet LVS®, and Castor oil.

Another embodiment comprises cyclosporin A, Marlowet R 11®, and Castor oil.

Another embodiment comprises cyclosporin A, Marlowet R 40®, and Castor oil.

Another embodiment comprises cyclosporin A, Mazol 80 MGK®, and Castor oil.

Another embodiment comprises cyclosporin A, Nonionic Emulsifier T-9®, and Castor oil.

Another embodiment comprises cyclosporin A, Oronal LCG®, and Castor oil.

Another embodiment comprises cyclosporin A, Polyderm PPI-CO-200®, and Castor oil.

Another embodiment comprises cyclosporin A, Polyderm PPI-CO-40®, and Castor oil.

Another embodiment comprises cyclosporin A, Rewoderm LI 520-70®, and Castor oil.

Another embodiment comprises cyclosporin A, Ritapeg 150 DS®, and Castor oil.

Another embodiment comprises cyclosporin A, Softigen 767®, and Castor oil.

Another embodiment comprises cyclosporin A, Surfactol 318®, and Castor oil.

Another embodiment comprises cyclosporin A, Surfactol 365®, and Castor oil.

Another embodiment comprises cyclosporin A, Syn Lube 107®, and Castor oil.

Another embodiment comprises cyclosporin A, Syn Lube 728®, and Castor oil.

Another embodiment comprises cyclosporin A, Syn Lube 1632H®, and Castor oil.

Another embodiment comprises cyclosporin A, Syn Lube 6277-A®, and Castor oil.

Another embodiment comprises cyclosporin A, T-Det C-20®, and Castor oil.

Another embodiment comprises cyclosporin A, T-Det C-40®, and Castor oil.

Another embodiment comprises cyclosporin A, Tally 100 Plus®, and Castor oil.

Another embodiment comprises cyclosporin A, Uniperol EL®, and Castor oil.

Another embodiment comprises cyclosporin A, a Fatty Ester, and Castor oil.

Another embodiment comprises cyclosporin A, Actralube-Syn 147®, and Castor oil.

Another embodiment comprises cyclosporin A, Atlas G-1556®, and Castor oil.

Another embodiment comprises cyclosporin A, Atlas G-1564®, and Castor oil.

Another embodiment comprises cyclosporin A, Atlasol Base Oil S®, and Castor oil.

Another embodiment comprises cyclosporin A, Base ML®, and Castor oil.

Another embodiment comprises cyclosporin A, Base MT®, and Castor oil.

Another embodiment comprises cyclosporin A, Cerasynt 303®, and Castor oil.

Another embodiment comprises cyclosporin A, Dermol 1012®, and Castor oil.

Another embodiment comprises cyclosporin A, Kemester 4000®, and Castor oil.

Another embodiment comprises cyclosporin A, Lactipol S®, and Castor oil.

Another embodiment comprises cyclosporin A, Magrabar PGO®, and Castor oil.

Another embodiment comprises cyclosporin A, Mayco Base BFO®, and Castor oil.

Another embodiment comprises cyclosporin A, Methyl Linoleate®, and Castor oil.

Another embodiment comprises cyclosporin A, Pationic 122A®, and Castor oil.

Another embodiment comprises cyclosporin A, Pationic 138C®, and Castor oil.

Another embodiment comprises cyclosporin A, Pationic CSL®, and Castor oil.

Another embodiment comprises cyclosporin A, Pationic ISL®, and Castor oil.

Another embodiment comprises cyclosporin A, Pationic SBL®, and Castor oil.

Another embodiment comprises cyclosporin A, Pationic SSL®, and Castor oil.

Another embodiment comprises cyclosporin A, Ritasol®, and Castor oil.

Another embodiment comprises cyclosporin A, Tego Alkanol CS 20®, and Castor oil.

Another embodiment comprises cyclosporin A, Tego Alkanol L23 P®, and Castor oil.

Another embodiment comprises cyclosporin A, Tego Alkanol S2®, and Castor oil.

Another embodiment comprises cyclosporin A, Tego Alkanol S20 P®, and Castor oil.

Another embodiment comprises cyclosporin A, Triemulsifier 600 MS®, and Castor oil.

Another embodiment comprises cyclosporin A, a Glycerol Esters, and Castor oil.

Another embodiment comprises cyclosporin A, Agro #9 Wint SBO®, and Castor oil.

Another embodiment comprises cyclosporin A, Ahcovel Base 700®, and Castor oil.

Another embodiment comprises cyclosporin A, Aldo HMS FG®, and Castor oil.

Another embodiment comprises cyclosporin A, Aldo MLD®, and Castor oil.

Another embodiment comprises cyclosporin A, Aldo MLD FG®, and Castor oil.

Another embodiment comprises cyclosporin A, Aldo MO FG®, and Castor oil.

Another embodiment comprises cyclosporin A, Aldo MS®, and Castor oil.

Another embodiment comprises cyclosporin A, Aldo MS FG®, and Castor oil.

Another embodiment comprises cyclosporin A, Aldo MS LG FG®, and Castor oil.

Another embodiment comprises cyclosporin A, Aldo MSD®, and Castor oil.

Another embodiment comprises cyclosporin A, Aldo MSD FG®, and Castor oil.

Another embodiment comprises cyclosporin A, Aldosperse O-20 FG®, and Castor oil.

Another embodiment comprises cyclosporin A, Aldosperse TS-20 FG®, and Castor oil.

Another embodiment comprises cyclosporin A, Aldosperse TS-40 FG®, and Castor oil.

Another embodiment comprises cyclosporin A, Arlacel 165®, and Castor oil.

Another embodiment comprises cyclosporin A, Arlacel 186®, and Castor oil.

Another embodiment comprises cyclosporin A, Capmul GMO®, and Castor oil.

Another embodiment comprises cyclosporin A, Capmul GMS®, and Castor oil.

Another embodiment comprises cyclosporin A, Caprol 3GO®, and Castor oil.

Another embodiment comprises cyclosporin A, Caprol 3GVS®, and Castor oil.

Another embodiment comprises cyclosporin A, Caprol 6G2S®, and Castor oil.

Another embodiment comprises cyclosporin A, Caprol 10G40®, and Castor oil.

Another embodiment comprises cyclosporin A, Caprol 10G100®, and Castor oil.

Another embodiment comprises cyclosporin A, Caprol ET®, and Castor oil.

Another embodiment comprises cyclosporin A, Caprol PGE860®, and Castor oil.

Another embodiment comprises cyclosporin A, Cerasynt 945®, and Castor oil.

Another embodiment comprises cyclosporin A, Cerasynt GMS®, and Castor oil.

Another embodiment comprises cyclosporin A, Cerasynt Q®, and Castor oil.

Another embodiment comprises cyclosporin A, Cerasynt SD®, and Castor oil.

Another embodiment comprises cyclosporin A, Cerasynt WM®, and Castor oil.

Another embodiment comprises cyclosporin A, Chemsperse 14®, and Castor oil.

Another embodiment comprises cyclosporin A, Cremophor GO-32®, and Castor oil.

Another embodiment comprises cyclosporin A, Cremophor GS11®, and Castor oil.

Another embodiment comprises cyclosporin A, Cremophor GS-32®, and Castor oil.

Another embodiment comprises cyclosporin A, Cutina KD-16®, and Castor oil.

Another embodiment comprises cyclosporin A, Dehymuls PGPH®, and Castor oil.

Another embodiment comprises cyclosporin A, Dermol DGDIS®, and Castor oil.

Another embodiment comprises cyclosporin A, Dermol DGMIS®, and Castor oil.

Another embodiment comprises cyclosporin A, Dermol G-76®, and Castor oil.

Another embodiment comprises cyclosporin A, Dermol G-7DI®, and Castor oil.

Another embodiment comprises cyclosporin A, Dermol NGDI®, and Castor oil.

Another embodiment comprises cyclosporin A, Dermolan GLH®, and Castor oil.

Another embodiment comprises cyclosporin A, Drewmulse GMO®, and Castor oil.

Another embodiment comprises cyclosporin A, Drewpol 3-5-M®, and Castor oil.

Another embodiment comprises cyclosporin A, Durlac 100 W®, and Castor oil.

Another embodiment comprises cyclosporin A, Dur-Lo®, and Castor oil.

Another embodiment comprises cyclosporin A, Dynasan 118®, and Castor oil.

Another embodiment comprises cyclosporin A, EC-25®, and Castor oil.

Another embodiment comprises cyclosporin A, EM 40®, and Castor oil.

Another embodiment comprises cyclosporin A, Emerest 2400®, and Castor oil.

Another embodiment comprises cyclosporin A, Emerest 2452®, and Castor oil.

Another embodiment comprises cyclosporin A, Empilan G-26®, and Castor oil.

Another embodiment comprises cyclosporin A, Genapol TSM®, and Castor oil.

Another embodiment comprises cyclosporin A, Hostacerin DGI®, and Castor oil.

Another embodiment comprises cyclosporin A, Hostacerin DGL®, and Castor oil.

Another embodiment comprises cyclosporin A, Hostacerin DGMS®, and Castor oil.

Another embodiment comprises cyclosporin A, Hostacerin DGSB®, and Castor oil.

Another embodiment comprises cyclosporin A, Ice No. 2®, and Castor oil.

Another embodiment comprises cyclosporin A, Imwitor 742®, and Castor oil.

Another embodiment comprises cyclosporin A, Imwitor 780 K®, and Castor oil.

Another embodiment comprises cyclosporin A, Imwitor 960 Flakes®, and Castor oil.

Another embodiment comprises cyclosporin A, Isolan GI 34®, and Castor oil.

Another embodiment comprises cyclosporin A, Isolan GO 33®, and Castor oil.

Another embodiment comprises cyclosporin A, Kemester 1000®, and Castor oil.

Another embodiment comprises cyclosporin A, Kemester 2000®, and Castor oil.

Another embodiment comprises cyclosporin A, Kemester 2000®, and Castor oil.

Another embodiment comprises cyclosporin A, Kemester 6000SE®, and Castor oil.

Another embodiment comprises cyclosporin A, Lamecreme DGE 18®, and Castor oil.

Another embodiment comprises cyclosporin A, Lexemul 515®, and Castor oil.

Another embodiment comprises cyclosporin A, Lexemul 561®, and Castor oil.

Another embodiment comprises cyclosporin A, Lexemul AR®, and Castor oil.

Another embodiment comprises cyclosporin A, Lexemul AS®, and Castor oil.

Another embodiment comprises cyclosporin A, Lexemul GDL®, and Castor oil.

Another embodiment comprises cyclosporin A, Lexemul T®, and Castor oil.

Another embodiment comprises cyclosporin A, Lipomulse 165®, and Castor oil.

Another embodiment comprises cyclosporin A, Lumulse GML K®, and Castor oil.

Another embodiment comprises cyclosporin A, Lumulse GMO K®, and Castor oil.

Another embodiment comprises cyclosporin A, Lumulse GMR K®, and Castor oil.

Another embodiment comprises cyclosporin A, Lumulse GMT K®, and Castor oil.

Another embodiment comprises cyclosporin A, Magrabar GMC®, and Castor oil.

Another embodiment comprises cyclosporin A, Magrabar GMO-CK®, and Castor oil.

Another embodiment comprises cyclosporin A, Magrabar MDG-5050®, and Castor oil.

Another embodiment comprises cyclosporin A, Magrabar PGO-315®, and Castor oil.

Another embodiment comprises cyclosporin A, Magrabar PGO-1010®, and Castor oil.

Another embodiment comprises cyclosporin A, Mazol 300K®, and Castor oil.

Another embodiment comprises cyclosporin A, Mazol GMO-K®, and Castor oil.

Another embodiment comprises cyclosporin A, Mazol GMS-K®, and Castor oil.

Another embodiment comprises cyclosporin A, Mazol PG031-K®, and Castor oil.

Another embodiment comprises cyclosporin A, Miglyol 812®, and Castor oil.

Another embodiment comprises cyclosporin A, Norfox 165C®, and Castor oil.

Another embodiment comprises cyclosporin A, Schercemol GMIS®, and Castor oil.

Another embodiment comprises cyclosporin A, Tegin®, and Castor oil.

Another embodiment comprises cyclosporin A, Tegin 4100 Pellets®, and Castor oil.

Another embodiment comprises cyclosporin A, Tegin M Pellets®, and Castor oil.

Another embodiment comprises cyclosporin A, Tegin OV®, and Castor oil.

Another embodiment comprises cyclosporin A, Teginacid H®, and Castor oil.

Another embodiment comprises cyclosporin A, Tego Cosmo P813®, and Castor oil.

Another embodiment comprises cyclosporin A, Wickenol 535®, and Castor oil.

Another embodiment comprises cyclosporin A, Witconol 14®, and Castor oil.

Another embodiment comprises cyclosporin A, Witconol 14®, and Castor oil.

Another embodiment comprises cyclosporin A, Witconol 14F®, and Castor oil.

Another embodiment comprises cyclosporin A, Witconol 18L®, and Castor oil.

Another embodiment comprises cyclosporin A, Witconol GOT®, and Castor oil.

Another embodiment comprises cyclosporin A, Witconol MST®, and Castor oil.

Another embodiment comprises cyclosporin A, Witconol RHT®, and Castor oil.

Another embodiment comprises cyclosporin A, a Glycol Ester, and Castor oil.

Another embodiment comprises cyclosporin A, Alkamuls 600 DO®, and Castor oil.

Another embodiment comprises cyclosporin A, Alkamuls SEG®, and Castor oil.

Another embodiment comprises cyclosporin A, Atlas EM-2®, and Castor oil.

Another embodiment comprises cyclosporin A, Cerasynt IP®, and Castor oil.

Another embodiment comprises cyclosporin A, Cerasynt M®, and Castor oil.

Another embodiment comprises cyclosporin A, Cerasynt MN®, and Castor oil.

Another embodiment comprises cyclosporin A, Cerasynt PA®, and Castor oil.

Another embodiment comprises cyclosporin A, Chemsperse EGDS®, and Castor oil.

Another embodiment comprises cyclosporin A, Chemsperse EGMS®, and Castor oil.

Another embodiment comprises cyclosporin A, Colonial Monolaurin®, and Castor oil.

Another embodiment comprises cyclosporin A, DeMuls SGE-95®, and Castor oil.

Another embodiment comprises cyclosporin A, Eccoterge 200®, and Castor oil.

Another embodiment comprises cyclosporin A, Emerest 2380®, and Castor oil.

Another embodiment comprises cyclosporin A, Ethox 2610®, and Castor oil.

Another embodiment comprises cyclosporin A, Ethox DO-9®, and Castor oil.

Another embodiment comprises cyclosporin A, Ethox DO-14®, and Castor oil.

Another embodiment comprises cyclosporin A, Ethox SO-9®, and Castor oil.

Another embodiment comprises cyclosporin A, Fizul MD-318®, and Castor oil.

Another embodiment comprises cyclosporin A, Genapol EGDS-VHP®, and Castor oil.

Another embodiment comprises cyclosporin A, Genapol TS Powder®, and Castor oil.

Another embodiment comprises cyclosporin A, Hostacerin WO®, and Castor oil.

Another embodiment comprises cyclosporin A, Inversol 140®, and Castor oil.

Another embodiment comprises cyclosporin A, Kemester 104®, and Castor oil.

Another embodiment comprises cyclosporin A, Kemester 205®, and Castor oil.

Another embodiment comprises cyclosporin A, Kemester 226®, and Castor oil.

Another embodiment comprises cyclosporin A, Kemester 5221SE®, and Castor oil.

Another embodiment comprises cyclosporin A, Kemester EGDS®, and Castor oil.

Another embodiment comprises cyclosporin A, Lexemul EGDS®, and Castor oil.

Another embodiment comprises cyclosporin A, Lexemul EGMS®, and Castor oil.

Another embodiment comprises cyclosporin A, Lexemul P®, and Castor oil.

Another embodiment comprises cyclosporin A, Lipo DGLS®, and Castor oil.

Another embodiment comprises cyclosporin A, Lipo EGDS®, and Castor oil.

Another embodiment comprises cyclosporin A, Lipo PGMS®, and Castor oil.

Another embodiment comprises cyclosporin A, Liposorb S-4®, and Castor oil.

Another embodiment comprises cyclosporin A, Liposorb TO-200, and Castor oil.

Another embodiment comprises cyclosporin A, Lumulse PGO®, and Castor oil.

Another embodiment comprises cyclosporin A, Mackester EGDS®, and Castor oil.

Another embodiment comprises cyclosporin A, Mackester EGMS®, and Castor oil.

Another embodiment comprises cyclosporin A, Mackester GSTP®, and Castor oil.

Another embodiment comprises cyclosporin A, Mackester Series®, and Castor oil.

Another embodiment comprises cyclosporin A, Magrabar PDG-50®, and Castor oil.

Another embodiment comprises cyclosporin A, Mapeg 6000 DS®, and Castor oil.

Another embodiment comprises cyclosporin A, Marlowet 4702®, and Castor oil.

Another embodiment comprises cyclosporin A, Monalube 305®, and Castor oil.

Another embodiment comprises cyclosporin A, Monalube 310®, and Castor oil.

Another embodiment comprises cyclosporin A, Monalube 315®, and Castor oil.

Another embodiment comprises cyclosporin A, Monalube 320®, and Castor oil.

Another embodiment comprises cyclosporin A, Monalube 325®, and Castor oil.

Another embodiment comprises cyclosporin A, Monalube 330®, and Castor oil.

Another embodiment comprises cyclosporin A, Naturechem PGHS®, and Castor oil.

Another embodiment comprises cyclosporin A, Polycastorol PLO-840®, and Castor oil.

Another embodiment comprises cyclosporin A, Polytex 10M®, and Castor oil.

Another embodiment comprises cyclosporin A, Ritasynt IP®, and Castor oil.

Another embodiment comprises cyclosporin A, Ross Chem PEG 600 DT®, and Castor oil.

Another embodiment comprises cyclosporin A, Schercemol PGMS®, and Castor oil.

Another embodiment comprises cyclosporin A, Sponto H-44C®, and Castor oil.

Another embodiment comprises cyclosporin A, Tegin G®, and Castor oil.

Another embodiment comprises cyclosporin A, Witbreak DGE-182®, and Castor oil.

Another embodiment comprises cyclosporin A, Witbreak DGE-182®, and Castor oil.

Another embodiment comprises cyclosporin A, Witbreak DRA-21®, and Castor oil.

Another embodiment comprises cyclosporin A, Witbreak DRA-21®, and Castor oil.

Another embodiment comprises cyclosporin A, Witbreak DRA-50®, and Castor oil.

Another embodiment comprises cyclosporin A, Witbreak DRA-50®, and Castor oil.

Another embodiment comprises cyclosporin A, Witconol F26-46®, and Castor oil.

Another embodiment comprises cyclosporin A, Witconol H-32®, and Castor oil.

Another embodiment comprises cyclosporin A, Witconol H-33®, and Castor oil.

Another embodiment comprises cyclosporin A, Witconol H-35A®, and Castor oil.

Another embodiment comprises cyclosporin A, Witconol RHP®, and Castor oil.

Another embodiment comprises cyclosporin A, a Lanolin-based Derivative, and Castor oil.

Another embodiment comprises cyclosporin A, Amerchol CAB®, and Castor oil.

Another embodiment comprises cyclosporin A, Amerchol L-101®, and Castor oil.

Another embodiment comprises cyclosporin A, Amerlate LFA-LO®, and Castor oil.

Another embodiment comprises cyclosporin A, Amerlate P®, and Castor oil.

Another embodiment comprises cyclosporin A, Barre Common Degras®, and Castor oil.

Another embodiment comprises cyclosporin A, Cholesterol NF®, and Castor oil.

Another embodiment comprises cyclosporin A, Crodalan AWS®, and Castor oil.

Another embodiment comprises cyclosporin A, Crodalan LA®, and Castor oil.

Another embodiment comprises cyclosporin A, Emery 1650®, and Castor oil.

Another embodiment comprises cyclosporin A, Emery 1650®, and Castor oil.

Another embodiment comprises cyclosporin A, Emery 1740®, and Castor oil.

Another embodiment comprises cyclosporin A, Emery 1740®, and Castor oil.

Another embodiment comprises cyclosporin A, Forlan 500®, and Castor oil.

Another embodiment comprises cyclosporin A, Forlan L®, and Castor oil.

Another embodiment comprises cyclosporin A, Laneto 50®, and Castor oil.

Another embodiment comprises cyclosporin A, Laneto 100®, and Castor oil.

Another embodiment comprises cyclosporin A, Laneto AWS®, and Castor oil.

Another embodiment comprises cyclosporin A, Lanfrax 1776®, and Castor oil.

Another embodiment comprises cyclosporin A, Lanfrax 1776®, and Castor oil.

Another embodiment comprises cyclosporin A, Lanogel 21®, and Castor oil.

Another embodiment comprises cyclosporin A, Lipolan®, and Castor oil.

Another embodiment comprises cyclosporin A, Lipolan 31®, and Castor oil.

Another embodiment comprises cyclosporin A, OHlan®, and Castor oil.

Another embodiment comprises cyclosporin A, Polychol 5®, and Castor oil.

Another embodiment comprises cyclosporin A, Polychol 15®, and Castor oil.

Another embodiment comprises cyclosporin A, Ritacetyl®, and Castor oil.

Another embodiment comprises cyclosporin A, Ritachol®, and Castor oil.

Another embodiment comprises cyclosporin A, Ritahydrox®, and Castor oil.

Another embodiment comprises cyclosporin A, Ritalafa®, and Castor oil.

Another embodiment comprises cyclosporin A, Ritalan®, and Castor oil.

Another embodiment comprises cyclosporin A, Ritalan AWS®, and Castor oil.

Another embodiment comprises cyclosporin A, Ritalan C®, and Castor oil.

Another embodiment comprises cyclosporin A, Ritawax®, and Castor oil.

Another embodiment comprises cyclosporin A, Ritawax AEO®, and Castor oil.

Another embodiment comprises cyclosporin A, Ritawax ALA®, and Castor oil.

Another embodiment comprises cyclosporin A, Solan/Solan 50/Super Solan®, and Castor oil.

Another embodiment comprises cyclosporin A, Super Hartolan/Hartolan®, and Castor oil.

Another embodiment comprises cyclosporin A, Supersat AWS-4®, and Castor oil.

Another embodiment comprises cyclosporin A, Supersat AWS-24®, and Castor oil.

Another embodiment comprises cyclosporin A, a Lecithin or Lecithin Derivative, and Castor oil.

Another embodiment comprises cyclosporin A, Alcolec®, and Castor oil.

Another embodiment comprises cyclosporin A, Lecithin®, and Castor oil.

Another embodiment comprises cyclosporin A, Lexin K®, and Castor oil.

Another embodiment comprises cyclosporin A, Natipide®, and Castor oil.

Another embodiment comprises cyclosporin A, a Lignin or Lignin Derivative, and Castor oil.

Another embodiment comprises cyclosporin A, Diwatex XP 9®, and Castor oil.

Another embodiment comprises cyclosporin A, Dynasperse LCD®, and Castor oil.

Another embodiment comprises cyclosporin A, Indulin SAL®, and Castor oil.

Another embodiment comprises cyclosporin A, Indulin W-1®, and Castor oil.

Another embodiment comprises cyclosporin A, Indulin W-5®, and Castor oil.

Another embodiment comprises cyclosporih A, Lignosol FTA®, and Castor oil.

Another embodiment comprises cyclosporin A, Lignosol SFX-65®, and Castor oil.

Another embodiment comprises cyclosporin A, Marasperse 52 CP®, and Castor oil.

Another embodiment comprises cyclosporin A, Marasperse AG®, and Castor oil.

Another embodiment comprises cyclosporin A, Marasperse CBOS-4®, and Castor oil.

Another embodiment comprises cyclosporin A, Ufoxane 2®, and Castor oil.

Another embodiment comprises cyclosporin A, a Methyl Ester, and Castor oil.

Another embodiment comprises cyclosporin A, E.B. Cleaner AK®, and Castor oil.

Another embodiment comprises cyclosporin A, Oleocal ME-70®, and Castor oil.

Another embodiment comprises cyclosporin A, Trade Name Company®, and Castor oil.

Another embodiment comprises cyclosporin A, Methyl Esters (cont'd)®, and Castor oil.

Another embodiment comprises cyclosporin A, Oleocal ME-92®, and Castor oil.

Another embodiment comprises cyclosporin A, Oleocal ME-112®, and Castor oil.

Another embodiment comprises cyclosporin A, Oleocal ME-130®, and Castor oil.

Another embodiment comprises cyclosporin A, a Monoglyceride or a Derivative thereof, and Castor oil.

Another embodiment comprises cyclosporin A, Dynacet 211®, and Castor oil.

Another embodiment comprises cyclosporin A, Hetsorb S-20®, and Castor oil.

Another embodiment comprises cyclosporin A, Imwitor 191®, and Castor oil.

Another embodiment comprises cyclosporin A, Imwitor 370®, and Castor oil.

Another embodiment comprises cyclosporin A, Imwitor 375®, and Castor oil.

Another embodiment comprises cyclosporin A, Imwitor 900®, and Castor oil.

Another embodiment comprises cyclosporin A, Imwitor 945®, and Castor oil.

Another embodiment comprises cyclosporin A, Imwitor 2020®, and Castor oil.

Another embodiment comprises cyclosporin A, Kemester 5500®, and Castor oil.

Another embodiment comprises cyclosporin A, Kemester 6000®, and Castor oil.

Another embodiment comprises cyclosporin A, Magrabar GMC®, and Castor oil.

Another embodiment comprises cyclosporin A, Magrabar GMO-CK®, and Castor oil.

Another embodiment comprises cyclosporin A, Magrabar GPC-10®, and Castor oil.

Another embodiment comprises cyclosporin A, Magrabar MDG-5050®, and Castor oil.

Another embodiment comprises cyclosporin A, Monalube 335®, and Castor oil.

Another embodiment comprises cyclosporin A, Monoglycerides Glyceryl Monestearate Archer Daniels Midland Company®, and Castor oil.

Another embodiment comprises cyclosporin A, Rita GMS®, and Castor oil.

Another embodiment comprises cyclosporin A, Ritamulse SCG®, and Castor oil.

Another embodiment comprises cyclosporin A, Softigen 701®, and Castor oil.

Another embodiment comprises cyclosporin A, Tally 100 Plus®, and Castor oil.

Another embodiment comprises cyclosporin A, a Polyethylene Glycol, and Castor oil.

Another embodiment comprises cyclosporin A, Emulgade PL 68/50®, and Castor oil.

Another embodiment comprises cyclosporin A, Lumulse PEG®, and Castor oil.

Another embodiment comprises cyclosporin A, Rhodasurf PEG-400®, and Castor oil.

Another embodiment comprises cyclosporin A, Rhodasurf PEG-600®, and Castor oil.

Another embodiment comprises cyclosporin A, Witconol PEG-400®, and Castor oil.

Another embodiment comprises cyclosporin A, a Polymeric Surfactant, and Castor oil.

Another embodiment comprises cyclosporin A, Acritamer PNC-EG®, and Castor oil.

Another embodiment comprises cyclosporin A, Ag-Rho DEP-775®, and Castor oil.

Another embodiment comprises cyclosporin A, APG 325N Glycoside®, and Castor oil.

Another embodiment comprises cyclosporin A, Aristoflex AVC®, and Castor oil.

Another embodiment comprises cyclosporin A, Aristoflex HMB®, and Castor oil.

Another embodiment comprises cyclosporin A, Burco NPS-225®, and Castor oil.

Another embodiment comprises cyclosporin A, Burco NPS-816®, and Castor oil.

Another embodiment comprises cyclosporin A, Chemccinate 5603®, and Castor oil.

Another embodiment comprises cyclosporin A, Cosmedia Guar C-261N®, and Castor oil.

Another embodiment comprises cyclosporin A, Gantrez S-95®, and Castor oil.

Another embodiment comprises cyclosporin A, Glucopon 220 UP®, and Castor oil.

Another embodiment comprises cyclosporin A, Glucopon 225 DK®, and Castor oil.

Another embodiment comprises cyclosporin A, Glucopon 425 N®, and Castor oil.

Another embodiment comprises cyclosporin A, Glucopon 600 UP®, and Castor oil.

Another embodiment comprises cyclosporin A, Glucopon 625 UP®, and Castor oil.

Another embodiment comprises cyclosporin A, Pemulen 1621®, and Castor oil.

Another embodiment comprises cyclosporin A, Pemulen 1622®, and Castor oil.

Another embodiment comprises cyclosporin A, Pemulen TR-1®, and Castor oil.

Another embodiment comprises cyclosporin A, Pemulen TR-2®, and Castor oil.

Another embodiment comprises cyclosporin A, Plantacare 818®, and Castor oil.

Another embodiment comprises cyclosporin A, Plantapon LGC Sorb®, and Castor oil.

Another embodiment comprises cyclosporin A, Plantaren 1200N®, and Castor oil.

Another embodiment comprises cyclosporin A, Plantaren 2000N®, and Castor oil.

Another embodiment comprises cyclosporin A, Viscolam AT 64®, and Castor oil.

Another embodiment comprises cyclosporin A, Viscolam AT 64P®, and Castor oil.

Another embodiment comprises cyclosporin A, Viscolam AT 100®, and Castor oil.

Another embodiment comprises cyclosporin A, Viscolam MAC 7®, and Castor oil.

Another embodiment comprises cyclosporin A, Viscolam SMC 20®, and Castor oil.

Another embodiment comprises cyclosporin A, Witbreak RTC-323®, and Castor oil.

Another embodiment comprises cyclosporin A, WSI 3700®, and Castor oil.

Another embodiment comprises cyclosporin A, a Propoxylated or Ethoxylated Fatty Acid, Alcohol, or Alkyl Phenol, and Castor oil.

Another embodiment comprises cyclosporin A, Antarox AA-60®, and Castor oil.

Another embodiment comprises cyclosporin A, Antarox LF-224®, and Castor oil.

Another embodiment comprises cyclosporin A, Burcomul DFE-45®, and Castor oil.

Another embodiment comprises cyclosporin A, Burcoterge LFE-1000®, and Castor oil.

Another embodiment comprises cyclosporin A, Chemal LF-25B®, and Castor oil.

Another embodiment comprises cyclosporin A, Chemal LF-40B®, and Castor oil.

Another embodiment comprises cyclosporin A, Dehypon LS-36®, and Castor oil.

Another embodiment comprises cyclosporin A, Dehypon LS-36®, and Castor oil.

Another embodiment comprises cyclosporin A, Dehypon LS-54®, and Castor oil.

Another embodiment comprises cyclosporin A, Dehypon LS-54®, and Castor oil.

Another embodiment comprises cyclosporin A, Delonic 100 VLF®, and Castor oil.

Another embodiment comprises cyclosporin A, Delonic LF-60 MOD®, and Castor oil.

Another embodiment comprises cyclosporin A, Empiderm B®, and Castor oil.

Another embodiment comprises cyclosporin A, Ethylan 1206®, and Castor oil.

Another embodiment comprises cyclosporin A, Ethylan NS-500K®, and Castor oil.

Another embodiment comprises cyclosporin A, Ethylan NS-500LQ®, and Castor oil.

Another embodiment comprises cyclosporin A, Genapol 1392®, and Castor oil.

Another embodiment comprises cyclosporin A, Genapol 2317®, and Castor oil.

Another embodiment comprises cyclosporin A, Genapol 26EP710®, and Castor oil.

Another embodiment comprises cyclosporin A, Genapol EP 1022®, and Castor oil.

Another embodiment comprises cyclosporin A, Genapol EP 1024®, and Castor oil.

Another embodiment comprises cyclosporin A, Genapol EP 6068®, and Castor oil.

Another embodiment comprises cyclosporin A, Genapol NP915®, and Castor oil.

Another embodiment comprises cyclosporin A, Kieralon MFB®, and Castor oil.

Another embodiment comprises cyclosporin A, Lumisolve CSA-80 V®, and Castor oil.

Another embodiment comprises cyclosporin A, Marlowet 5001®, and Castor oil.

Another embodiment comprises cyclosporin A, Marlox FK 64®, and Castor oil.

Another embodiment comprises cyclosporin A, Marlox MO 124®, and Castor oil.

Another embodiment comprises cyclosporin A, Marlox S 58®, and Castor oil.

Another embodiment comprises cyclosporin A, Nonatell 1003®, and Castor oil.

Another embodiment comprises cyclosporin A, Nonatell 1038®, and Castor oil.

Another embodiment comprises cyclosporin A, Nonatell 1052®, and Castor oil.

Another embodiment comprises cyclosporin A, Nonatell 1061®, and Castor oil.

Another embodiment comprises cyclosporin A, Nonatell 1075®, and Castor oil.

Another embodiment comprises cyclosporin A, Nonatell 1088®, and Castor oil.

Another embodiment comprises cyclosporin A, Nonatell 1123®, and Castor oil.

Another embodiment comprises cyclosporin A, Nonatell 1153®, and Castor oil.

Another embodiment comprises cyclosporin A, Nonatell 1161®, and Castor oil.

Another embodiment comprises cyclosporin A, Nonatell 1172®, and Castor oil.

Another embodiment comprises cyclosporin A, Nonatell 1181®, and Castor oil.

Another embodiment comprises cyclosporin A, Norfox 36®, and Castor oil.

Another embodiment comprises cyclosporin A, Procetyl AWS®, and Castor oil.

Another embodiment comprises cyclosporin A, Sandoxylate SX 412®, and Castor oil.

Another embodiment comprises cyclosporin A, Sandoxylate SX 418®, and Castor oil.

Another embodiment comprises cyclosporin A, Surfonic JL-80X®, and Castor oil.

Another embodiment comprises cyclosporin A, Surfonic JL-80X-B1®, and Castor oil.

Another embodiment comprises cyclosporin A, Surfonic L4-29X®, and Castor oil.

Another embodiment comprises cyclosporin A, Surfonic LF®, and Castor oil.

Another embodiment comprises cyclosporin A, T-Det A826®, and Castor oil.

Another embodiment comprises cyclosporin A, T-Det LF-416®, and Castor oil.

Another embodiment comprises cyclosporin A, Tergitol Min-Foam 1X®, and Castor oil.

Another embodiment comprises cyclosporin A, Tergitol Min-Foam 2X®, and Castor oil.

Another embodiment comprises cyclosporin A, Triton CF-21®, and Castor oil.

Another embodiment comprises cyclosporin A, Triton CF-76®, and Castor oil.

Another embodiment comprises cyclosporin A, Triton XL-80N®, and Castor oil.

Another embodiment comprises cyclosporin A, Witconol NS-98®, and Castor oil.

Another embodiment comprises cyclosporin A, Witconol NS-108LQ®, and Castor oil.

Another embodiment comprises cyclosporin A, Witconol NS-145®, and Castor oil.

Another embodiment comprises cyclosporin A, Witconol NS-179®, and Castor oil.

Another embodiment comprises cyclosporin A, a Protein-based Surfactant, and Castor oil.

Another embodiment comprises cyclosporin A, AminoFoam W®, and Castor oil.

Another embodiment comprises cyclosporin A, Amiter LGOD-2®, and Castor oil.

Another embodiment comprises cyclosporin A, Amiter LGS-2®, and Castor oil.

Another embodiment comprises cyclosporin A, Amiter LGS-5®, and Castor oil.

Another embodiment comprises cyclosporin A, Lamepon S®, and Castor oil.

Another embodiment comprises cyclosporin A, Lamepon S®, and Castor oil.

Another embodiment comprises cyclosporin A, Maypon 4C®, and Castor oil.

Another embodiment comprises cyclosporin A, May-Tein C®, and Castor oil.

Another embodiment comprises cyclosporin A, May-Tein CT®, and Castor oil.

Another embodiment comprises cyclosporin A, May-Tein KTS®, and Castor oil.

Another embodiment comprises cyclosporin A, May-Tein SY®, and Castor oil.

Another embodiment comprises cyclosporin A, Plantapon S®, and Castor oil.

Another embodiment comprises cyclosporin A, Proteol APL®, and Castor oil.

Another embodiment comprises cyclosporin A, Proteol OAT®, and Castor oil.

Another embodiment comprises cyclosporin A, Pyroter CPI-40®, and Castor oil.

Another embodiment comprises cyclosporin A, Pyroter GPI-25®, and Castor oil.

Another embodiment comprises cyclosporin A, Supro-Tein S®, and Castor oil.

Another embodiment comprises cyclosporin A, Supro-Tein V®, and Castor oil.

Another embodiment comprises cyclosporin A, ® and Castor oil.

Another embodiment comprises cyclosporin A, a Sarcosine Derivative®, and Castor oil.

Another embodiment comprises cyclosporin A, Crodasinic LS-30®, and Castor oil.

Another embodiment comprises cyclosporin A, Vanseal CS®, and Castor oil.

Another embodiment comprises cyclosporin A, Vanseal LS®, and Castor oil.

Another embodiment comprises cyclosporin A, Vanseal MS®, and Castor oil.

Another embodiment comprises cyclosporin A, Vanseal NACS-30®, and Castor oil.

Another embodiment comprises cyclosporin A, Vanseal NALS-95®, and Castor oil.

Another embodiment comprises cyclosporin A, Vanseal OS®, and Castor oil.

Another embodiment comprises cyclosporin A, ® and Castor oil.

Another embodiment comprises cyclosporin A, a Silicone-based Surfactant®, and Castor oil.

Another embodiment comprises cyclosporin A, Abil-B-9950®, and Castor oil.

Another embodiment comprises cyclosporin A, Abil Care 85®, and Castor oil.

Another embodiment comprises cyclosporin A, Abil EM 90®, and Castor oil.

Another embodiment comprises cyclosporin A, Abil EM 97®, and Castor oil.

Another embodiment comprises cyclosporin A, Abil WE-09®, and Castor oil.

Another embodiment comprises cyclosporin A, Dow Corning 1248 Fluid®, and Castor oil.

Another embodiment comprises cyclosporin A, Dow Corning 3225C®, and Castor oil.

Another embodiment comprises cyclosporin A, Dow Corning 5200®, and Castor oil.

Another embodiment comprises cyclosporin A, Dow Corning Q4-3667®, and Castor oil.

Another embodiment comprises cyclosporin A, Monasil PCA®, and Castor oil.

Another embodiment comprises cyclosporin A, Monasil PDM®, and Castor oil.

Another embodiment comprises cyclosporin A, Monasil PLN®, and Castor oil.

Another embodiment comprises cyclosporin A, Polyderm PPI-SI-WS®, and Castor oil.

Another embodiment comprises cyclosporin A, Troysol 380W®, and Castor oil.

Another embodiment comprises cyclosporin A, Troysol S366®, and Castor oil.

Another embodiment comprises cyclosporin A, a Sorbitan Derivative, and Castor oil.

Another embodiment comprises cyclosporin A, Alkamuls SML®, and Castor oil.

Another embodiment comprises cyclosporin A, Alkamuls SMO®, and Castor oil.

Another embodiment comprises cyclosporin A, Alkamuls STO®, and Castor oil.

Another embodiment comprises cyclosporin A, Arlacel 20®, and Castor oil.

Another embodiment comprises cyclosporin A, Arlacel 40®, and Castor oil.

Another embodiment comprises cyclosporin A, Arlacel 60®, and Castor oil.

Another embodiment comprises cyclosporin A, Arlacel 80®, and Castor oil.

Another embodiment comprises cyclosporin A, Arlacel C®, and Castor oil.

Another embodiment comprises cyclosporin A, Armul 21®, and Castor oil.

Another embodiment comprises cyclosporin A, Atlox 80®, and Castor oil.

Another embodiment comprises cyclosporin A, Atlox 847®, and Castor oil.

Another embodiment comprises cyclosporin A, Atlox 1045A®, and Castor oil.

Another embodiment comprises cyclosporin A, Canarcel 20®, and Castor oil.

Another embodiment comprises cyclosporin A, Canarcel 60®, and Castor oil.

Another embodiment comprises cyclosporin A, Canarcel 80®, and Castor oil.

Another embodiment comprises cyclosporin A, Canarcel TW 20®, and Castor oil.

Another embodiment comprises cyclosporin A, Canarcel TW 60®, and Castor oil.

Another embodiment comprises cyclosporin A, Canarcel TW 80®, and Castor oil.

Another embodiment comprises cyclosporin A, Coladet BSB-P®, and Castor oil.

Another embodiment comprises cyclosporin A, Customulse O-20®, and Castor oil.

Another embodiment comprises cyclosporin A, Dehymuls E®, and Castor oil.

Another embodiment comprises cyclosporin A, DeSotan SMO®, and Castor oil.

Another embodiment comprises cyclosporin A, DeSotan SMO-20®, and Castor oil.

Another embodiment comprises cyclosporin A, DeSotan SMT®, and Castor oil.

Another embodiment comprises cyclosporin A, DeSotan SMT-200, and Castor oil.

Another embodiment comprises cyclosporin A, Durfax 60®, and Castor oil.

Another embodiment comprises cyclosporin A, Durfax 65®, and Castor oil.

Another embodiment comprises cyclosporin A, Durfax 80®, and Castor oil.

Another embodiment comprises cyclosporin A, Durtan 60®, and Castor oil.

Another embodiment comprises cyclosporin A, Durtan 65®, and Castor oil.

Another embodiment comprises cyclosporin A, Liposorb L®, and Castor oil.

Another embodiment comprises cyclosporin A, Liposorb L-10®, and Castor oil.

Another embodiment comprises cyclosporin A, Liposorb L-20®, and Castor oil.

Another embodiment comprises cyclosporin A, Liposorb O®, and Castor oil.

Another embodiment comprises cyclosporin A, Liposorb O-20®, and Castor oil.

Another embodiment comprises cyclosporin A, Liposorb P®, and Castor oil.

Another embodiment comprises cyclosporin A, Liposorb P-20®, and Castor oil.

Another embodiment comprises cyclosporin A, Liposorb S®, and Castor oil.

Another embodiment comprises cyclosporin A, Liposorb S-20®, and Castor oil.

Another embodiment comprises cyclosporin A, Liposorb SQO®, and Castor oil.

Another embodiment comprises cyclosporin A, Liposorb TO®, and Castor oil.

Another embodiment comprises cyclosporin A, Liposorb TS®, and Castor oil.

Another embodiment comprises cyclosporin A, Liposorb TS-20®, and Castor oil.

Another embodiment comprises cyclosporin A, Lumisorb PS®, and Castor oil.

Another embodiment comprises cyclosporin A, Lumisorb SMO (T)®, and Castor oil.

Another embodiment comprises cyclosporin A, Lumisorb SMS K®, and Castor oil.

Another embodiment comprises cyclosporin A, Lumisorb SSO®, and Castor oil.

Another embodiment comprises cyclosporin A, Lumisorb STS K®, and Castor oil.

Another embodiment comprises cyclosporin A, Lumisorb STT®, and Castor oil.

Another embodiment comprises cyclosporin A, Magrabar SMO®, and Castor oil.

Another embodiment comprises cyclosporin A, Magrabar SMO-VEG®, and Castor oil.

Another embodiment comprises cyclosporin A, Magrabar SMT®, and Castor oil.

Another embodiment comprises cyclosporin A, Magrabar STO®, and Castor oil.

Another embodiment comprises cyclosporin A, Miracare BC-27®, and Castor oil.

Another embodiment comprises cyclosporin A, Ritabate 20®, and Castor oil.

Another embodiment comprises cyclosporin A, Ritabate 40®, and Castor oil.

Another embodiment comprises cyclosporin A, Ritabate 60®, and Castor oil.

Another embodiment comprises cyclosporin A, Ritabate 80®, and Castor oil.

Another embodiment comprises cyclosporin A, T-Maz®, and Castor oil.

Another embodiment comprises cyclosporin A, Tego SML®, and Castor oil.

Another embodiment comprises cyclosporin A, Tego SML 20®, and Castor oil.

Another embodiment comprises cyclosporin A, Tego SMO 80 V®, and Castor oil.

Another embodiment comprises cyclosporin A, Tego SMO V®, and Castor oil.

Another embodiment comprises cyclosporin A, Tego SMS®, and Castor oil.

Another embodiment comprises cyclosporin A, Tego STO V®, and Castor oil.

Another embodiment comprises cyclosporin A, Tween 21®, and Castor oil.

Another embodiment comprises cyclosporin A, Tween 40®, and Castor oil.

Another embodiment comprises cyclosporin A, Tween 60®, and Castor oil.

Another embodiment comprises cyclosporin A, Tween 60 K®, and Castor oil.

Another embodiment comprises cyclosporin A, Tween 61®, and Castor oil.

Another embodiment comprises cyclosporin A, Tween 65®, and Castor oil.

Another embodiment comprises cyclosporin A, Tween 80®, and Castor oil.

Another embodiment comprises cyclosporin A, Tween 80 K®, and Castor oil.

Another embodiment comprises cyclosporin A, Tween 81®, and Castor oil.

Another embodiment comprises cyclosporin A, Tween 85®, and Castor oil.

Another embodiment comprises cyclosporin A, a Sucrose or Glucose Ester, or Derivative thereof, and Castor oil.

Another embodiment comprises cyclosporin A, DeSulf GOS-P-60WCG®, and Castor oil.

Another embodiment comprises cyclosporin A, Glucam E-20 Distearate®, and Castor oil.

Another embodiment comprises cyclosporin A, Glucamate DOE-120®, and Castor oil.

Another embodiment comprises cyclosporin A, Glucamate SSE-20®, and Castor oil.

Another embodiment comprises cyclosporin A, Glucate DO®, and Castor oil.

Another embodiment comprises cyclosporin A, Glucate SS®, and Castor oil.

Another embodiment comprises cyclosporin A, Glucopon 425 UP®, and Castor oil.

Another embodiment comprises cyclosporin A, Isolan IS®, and Castor oil.

Another embodiment comprises cyclosporin A, Mazon 40®, and Castor oil.

Another embodiment comprises cyclosporin A, Montanov 82®, and Castor oil.

Another embodiment comprises cyclosporin A, Montanov 202®, and Castor oil.

Another embodiment comprises cyclosporin A, Montanov S®, and Castor oil.

Another embodiment comprises cyclosporin A, Rheozan®, and Castor oil.

Another embodiment comprises cyclosporin A, Simulsol AS 48®, and Castor oil.

Another embodiment comprises cyclosporin A, Simulsol SL 4®, and Castor oil.

Another embodiment comprises cyclosporin A, Simulsol SL 10®, and Castor oil.

Another embodiment comprises cyclosporin A, Simulsol SL 11W®, and Castor oil.

Another embodiment comprises cyclosporin A, Simulsol SL 55®, and Castor oil.

Another embodiment comprises cyclosporin A, Suga Nate 100 and 160®, and Castor oil.

Another embodiment comprises cyclosporin A, Tego Care 450®, and Castor oil.

Another embodiment comprises cyclosporin A, Tego Care CG 90®, and Castor oil.

Another embodiment comprises cyclosporin A, Tego Care PS®, and Castor oil.

Another embodiment comprises cyclosporin A, Tegosoft PSE 141 G®, and Castor oil.

Another embodiment comprises cyclosporin A, Tegotens G 826®, and Castor oil.

Another embodiment comprises cyclosporin A, Triton BG-10 (70%)®, and Castor oil.

Another embodiment comprises cyclosporin A, Triton CG-110 (60%)®, and Castor oil.

Another embodiment comprises cyclosporin A, Wickenol 545®, and Castor oil.

Another embodiment comprises cyclosporin A, an alcohol, and Clove oil.

Another embodiment comprises cyclosporin A, Diglycero®, and Clove oil.

Another embodiment comprises cyclosporin A, Hetoxide GT-80®, and Clove oil.

Another embodiment comprises cyclosporin A, Lexemul BEO®, and Clove oil.

Another embodiment comprises cyclosporin A, Polyglycerol-3®, and Clove oil.

Another embodiment comprises cyclosporin A, Redicote E Series®, and Clove oil.

Another embodiment comprises cyclosporin A, Simulsol OX 1005L®, and Clove oil.

Another embodiment comprises cyclosporin A, Stanfax 567®, and Clove oil.

Another embodiment comprises cyclosporin A, TA-1618®, and Clove oil.

Another embodiment comprises cyclosporin A, Witconol H-31A®, and Clove oil.

Another embodiment comprises cyclosporin A, an amine oxide, and Clove oil.

Another embodiment comprises cyclosporin A, AO-405®, and Clove oil.

Another embodiment comprises cyclosporin A, AO-455®, and Clove oil.

Another embodiment comprises cyclosporin A, AO 728 Special®, and Clove oil.

Another embodiment comprises cyclosporin A, Barlox 12®, and Clove oil.

Another embodiment comprises cyclosporin A, Barlox 14®, and Clove oil.

Another embodiment comprises cyclosporin A, Burcoxide Lo®, and Clove oil.

Another embodiment comprises cyclosporin A, Caloxamine LO®, and Clove oil.

Another embodiment comprises cyclosporin A, Chemoxide CAW®, and Clove oil.

Another embodiment comprises cyclosporin A, Chemoxide LM-30®, and Clove oil.

Another embodiment comprises cyclosporin A, Chemoxide LO®, and Clove oil.

Another embodiment comprises cyclosporin A, Chemoxide MO®, and Clove oil.

Another embodiment comprises cyclosporin A, Colalux CAO-35®, and Clove oil.

Another embodiment comprises cyclosporin A, Colalux LO®, and Clove oil.

Another embodiment comprises cyclosporin A, DeMox CAPO®, and Clove oil.

Another embodiment comprises cyclosporin A, DeMox CSG-30®, and Clove oil.

Another embodiment comprises cyclosporin A, DeMox LAO®, and Clove oil.

Another embodiment comprises cyclosporin A, Emcol L®, and Clove oil.

Another embodiment comprises cyclosporin A, Empigen OB®, and Clove oil.

Another embodiment comprises cyclosporin A, Empigen OS/A®, and Clove oil.

Another embodiment comprises cyclosporin A, Foamox CDO®, and Clove oil.

Another embodiment comprises cyclosporin A, Foamox DMM®, and Clove oil.

Another embodiment comprises cyclosporin A, Foamox DMS®, and Clove oil.

Another embodiment comprises cyclosporin A, Genaminox KC®, and Clove oil.

Another embodiment comprises cyclosporin A, Genaminox LA®, and Clove oil.

Another embodiment comprises cyclosporin A, Hartofoam SAO®, and Clove oil.

Another embodiment comprises cyclosporin A, Hartox DMCD®, and Clove oil.

Another embodiment comprises cyclosporin A, Lipowax DAT®, and Clove oil.

Another embodiment comprises cyclosporin A, Lipowax PB Pastilles®, and Clove oil.

Another embodiment comprises cyclosporin A, Mackamine C8®, and Clove oil.

Another embodiment comprises cyclosporin A, Mackamine C10®, and Clove oil.

Another embodiment comprises cyclosporin A, Mackamine C14®, and Clove oil.

Another embodiment comprises cyclosporin A, Mackamine CAO®, and Clove oil.

Another embodiment comprises cyclosporin A, Mackamine CO®, and Clove oil.

Another embodiment comprises cyclosporin A, Mackamine LO®, and Clove oil.

Another embodiment comprises cyclosporin A, Mackamine O2®, and Clove oil.

Another embodiment comprises cyclosporin A, Mackamine SAO®, and Clove oil.

Another embodiment comprises cyclosporin A, Mackamine SO®, and Clove oil.

Another embodiment comprises cyclosporin A, Mazox KCAO®, and Clove oil.

Another embodiment comprises cyclosporin A, Monalac MO®, and Clove oil.

Another embodiment comprises cyclosporin A, Norfox LDA®, and Clove oil.

Another embodiment comprises cyclosporin A, Rhodamox LO®, and Clove oil.

Another embodiment comprises cyclosporin A, Schercamox C-AA®, and Clove oil.

Another embodiment comprises cyclosporin A, Schercamox DMA®, and Clove oil.

Another embodiment comprises cyclosporin A, Schercamox DML®, and Clove oil.

Another embodiment comprises cyclosporin A, Schercamox DMM®, and Clove oil.

Another embodiment comprises cyclosporin A, Schercamox DMS®, and Clove oil.

Another embodiment comprises cyclosporin A, Tegotens DO®, and Clove oil.

Another embodiment comprises cyclosporin A, Tomah AO-14-2®, and Clove oil.

Another embodiment comprises cyclosporin A, Triaminox CDO®, and Clove oil.

Another embodiment comprises cyclosporin A, a block polymer, and Clove oil.

Another embodiment comprises cyclosporin A, AL 2070®, and Clove oil.

Another embodiment comprises cyclosporin A, Antarox 17-R-2®, and Clove oil.

Another embodiment comprises cyclosporin A, Antarox 25-R-2®, and Clove oil.

Another embodiment comprises cyclosporin A, Antarox 31-R-1®, and Clove oil.

Another embodiment comprises cyclosporin A, Antarox P-84®, and Clove oil.

Another embodiment comprises cyclosporin A, Antarox P-104/H®, and Clove oil.

Another embodiment comprises cyclosporin A, Arnox BP-Series®, and Clove oil.

Another embodiment comprises cyclosporin A, Chemonic 435®, and Clove oil.

Another embodiment comprises cyclosporin A, Chemonic D-25®, and Clove oil.

Another embodiment comprises cyclosporin A, Chemonic PL Series®, and Clove oil.

Another embodiment comprises cyclosporin A, Ethox L-121®, and Clove oil.

Another embodiment comprises cyclosporin A, Ethox L-122®, and Clove oil.

Another embodiment comprises cyclosporin A, Genapol PF-10®, and Clove oil.

Another embodiment comprises cyclosporin A, Genapol PF-20®, and Clove oil.

Another embodiment comprises cyclosporin A, Genapol PF-40A®, and Clove oil.

Another embodiment comprises cyclosporin A, Norox 2-LF®, and Clove oil.

Another embodiment comprises cyclosporin A, Simulsol NW 342®, and Clove oil.

Another embodiment comprises cyclosporin A, T-Det BP-1®, and Clove oil.

Another embodiment comprises cyclosporin A, T-Det XD®, and Clove oil.

Another embodiment comprises cyclosporin A, T-Det XH®, and Clove oil.

Another embodiment comprises cyclosporin A, Triton CF-32®, and Clove oil.

Another embodiment comprises cyclosporin A, Witconol 171®, and Clove oil.

Another embodiment comprises cyclosporin A, Witconol 324®, and Clove oil.

Another embodiment comprises cyclosporin A, Witconol 324D®, and Clove oil.

Another embodiment comprises cyclosporin A, Witconol PD-2000®, and Clove oil.

Another embodiment comprises cyclosporin A, Emcol CN-6®, and Clove oil.

Another embodiment comprises cyclosporin A, Ethcarb®, and Clove oil.

Another embodiment comprises cyclosporin A, Gemtex WNT-Conc®, and Clove oil.

Another embodiment comprises cyclosporin A, Incrodet TD7-C®, and Clove oil.

Another embodiment comprises cyclosporin A, Marlinat CM 105/80®, and Clove oil.

Another embodiment comprises cyclosporin A, Marlowet 1072®, and Clove oil.

Another embodiment comprises cyclosporin A, Marlowet 4530®, and Clove oil.

Another embodiment comprises cyclosporin A, Marlowet 4530 LF®, and Clove oil.

Another embodiment comprises cyclosporin A, Marlowet 4534®, and Clove oil.

Another embodiment comprises cyclosporin A, Marlowet 4538®, and Clove oil.

Another embodiment comprises cyclosporin A, Marlowet 4539®, and Clove oil.

Another embodiment comprises cyclosporin A, Marlowet 4539 LF®, and Clove oil.

Another embodiment comprises cyclosporin A, Marlowet 4541®, and Clove oil.

Another embodiment comprises cyclosporin A, Miranate LEC-80®, and Clove oil.

Another embodiment comprises cyclosporin A, Sandopan B®, and Clove oil.

Another embodiment comprises cyclosporin A, Sandopan B Modified®, and Clove oil.

Another embodiment comprises cyclosporin A, Sandopan LS-24 Gel®, and Clove oil.

Another embodiment comprises cyclosporin A, Surfine T-A®, and Clove oil.

Another embodiment comprises cyclosporin A, a Carboxylic Acid/Fatty Acid, and Clove oil.

Another embodiment comprises cyclosporin A, Colaterge RAM®, and Clove oil.

Another embodiment comprises cyclosporin A, Colatrope INC®, and Clove oil.

Another embodiment comprises cyclosporin A, Crodacid B®, and Clove oil.

Another embodiment comprises cyclosporin A, DeTrope CA-100®, and Clove oil.

Another embodiment comprises cyclosporin A, Latol MTO®, and Clove oil.

Another embodiment comprises cyclosporin A, Lumulse CC-33 K®, and Clove oil.

Another embodiment comprises cyclosporin A, Mulls 2218®, and Clove oil.

Another embodiment comprises cyclosporin A, OL-6000, and Clove oil.

Another embodiment comprises cyclosporin A, OL-800®, and Clove oil.

Another embodiment comprises cyclosporin A, R-910®, and Clove oil.

Another embodiment comprises cyclosporin A, S-210®, and Clove oil.

Another embodiment comprises cyclosporin A, Sandopan DTC Acid®, and Clove oil.

Another embodiment comprises cyclosporin A, Sandopan LS 24 N®, and Clove oil.

Another embodiment comprises cyclosporin A, Sandopan MA-18®, and Clove oil.

Another embodiment comprises cyclosporin A, an Ethoxylated Alcohol, and Clove oil.

Another embodiment comprises cyclosporin A, Adsee 799®, and Clove oil.

Another embodiment comprises cyclosporin A, Adsee 799®, and Clove oil.

Another embodiment comprises cyclosporin A, Alfonic 610-3.5®, and Clove oil.

Another embodiment comprises cyclosporin A, Alfonic 810-2®, and Clove oil.

Another embodiment comprises cyclosporin A, Alfonic 810-6®, and Clove oil.

Another embodiment comprises cyclosporin A, Alfonic 1012-3®, and Clove oil.

Another embodiment comprises cyclosporin A, Alfonic 1012-5®, and Clove oil.

Another embodiment comprises cyclosporin A, Alfonic 1216CO-1.5®, and Clove oil.

Another embodiment comprises cyclosporin A, Alfonic 1216CO-7®, and Clove oil.

Another embodiment comprises cyclosporin A, Alfonic 1412-3®, and Clove oil.

Another embodiment comprises cyclosporin A, Alfonic 1412-7®, and Clove oil.

Another embodiment comprises cyclosporin A, Arlasolve 200®, and Clove oil.

Another embodiment comprises cyclosporin A, Arlasolve 200 Liquid®, and Clove oil.

Another embodiment comprises cyclosporin A, Armix 180-C®, and Clove oil.

Another embodiment comprises cyclosporin A, Armix 183®, and Clove oil.

Another embodiment comprises cyclosporin A, Armul 2404®, and Clove oil.

Another embodiment comprises cyclosporin A, Armul 2404®, and Clove oil.

Another embodiment comprises cyclosporin A, Atlas EMJ-C®, and Clove oil.

Another embodiment comprises cyclosporin A, Atlas G-2109®, and Clove oil.

Another embodiment comprises cyclosporin A, Atlas G-3886®, and Clove oil.

Another embodiment comprises cyclosporin A, Atlas G-3890®, and Clove oil.

Another embodiment comprises cyclosporin A, Bio Soft E-200®, and Clove oil.

Another embodiment comprises cyclosporin A, Bio Soft E-300®, and Clove oil.

Another embodiment comprises cyclosporin A, Bio Soft E-400®, and Clove oil.

Another embodiment comprises cyclosporin A, Bio Soft EN 600®, and Clove oil.

Another embodiment comprises cyclosporin A, Bio Soft TD-400®, and Clove oil.

Another embodiment comprises cyclosporin A, Bio Soft TD-630®, and Clove oil.

Another embodiment comprises cyclosporin A, Brij 30®, and Clove oil.

Another embodiment comprises cyclosporin A, Brij 52®, and Clove oil.

Another embodiment comprises cyclosporin A, Brij 56®, and Clove oil.

Another embodiment comprises cyclosporin A, Brij 58®, and Clove oil.

Another embodiment comprises cyclosporin A, Brij 72®, and Clove oil.

Another embodiment comprises cyclosporin A, Brij 76®, and Clove oil.

Another embodiment comprises cyclosporin A, Brij 78®, and Clove oil.

Another embodiment comprises cyclosporin A, Brij 93®, and Clove oil.

Another embodiment comprises cyclosporin A, Brij 97®, and Clove oil.

Another embodiment comprises cyclosporin A, Brij 98®, and Clove oil.

Another embodiment comprises cyclosporin A, Brij 700®, and Clove oil.

Another embodiment comprises cyclosporin A, Brij 700 S®, and Clove oil.

Another embodiment comprises cyclosporin A, Brij 721®, and Clove oil.

Another embodiment comprises cyclosporin A, Brij 721 S®, and Clove oil.

Another embodiment comprises cyclosporin A, Burcoterge CDG®, and Clove oil.

Another embodiment comprises cyclosporin A, Canasol AT 600®, and Clove oil.

Another embodiment comprises cyclosporin A, Canasol AT 800®, and Clove oil.

Another embodiment comprises cyclosporin A, Canasol AT 1200®, and Clove oil.

Another embodiment comprises cyclosporin A, Canasol BJ 35®, and Clove oil.

Another embodiment comprises cyclosporin A, Canasol BJ 36®, and Clove oil.

Another embodiment comprises cyclosporin A, Canasol BJ 52®, and Clove oil.

Another embodiment comprises cyclosporin A, Canasol BJ 58®, and Clove oil.

Another embodiment comprises cyclosporin A, Canasol BJ 72®, and Clove oil.

Another embodiment comprises cyclosporin A, Canasol BJ 78®, and Clove oil.

Another embodiment comprises cyclosporin A, Canasol BJ 98®, and Clove oil.

Another embodiment comprises cyclosporin A, Canasol BJ 307®, and Clove oil.

Another embodiment comprises cyclosporin A, Cerfak 1400®, and Clove oil.

Another embodiment comprises cyclosporin A, Cetomacrogol 1000 BP®, and Clove oil.

Another embodiment comprises cyclosporin A, Chemonic C-2®, and Clove oil.

Another embodiment comprises cyclosporin A, Chemonic C-10®, and Clove oil.

Another embodiment comprises cyclosporin A, Chemonic C-20®, and Clove oil.

Another embodiment comprises cyclosporin A, Chemonic CT-12®, and Clove oil.

Another embodiment comprises cyclosporin A, Chemonic CT-20®, and Clove oil.

Another embodiment comprises cyclosporin A, Chemonic CT-30®, and Clove oil.

Another embodiment comprises cyclosporin A, Chembnic CT-55®, and Clove oil.

Another embodiment comprises cyclosporin A, Chemonic G-7®, and Clove oil.

Another embodiment comprises cyclosporin A, Chemonic G-26®, and Clove oil.

Another embodiment comprises cyclosporin A, Chemonic L-4®, and Clove oil.

Another embodiment comprises cyclosporin A, Chemonic L-7®, and Clove oil.

Another embodiment comprises cyclosporin A, Chemonic L-12®, and Clove oil.

Another embodiment comprises cyclosporin A, Chemonic L-23®, and Clove oil.

Another embodiment comprises cyclosporin A, Chemonic O-2®, and Clove oil.

Another embodiment comprises cyclosporin A, Chemonic O-5®, and Clove oil.

Another embodiment comprises cyclosporin A, Chemonic O-10®, and Clove oil.

Another embodiment comprises cyclosporin A, Chemonic O-20®, and Clove oil.

Another embodiment comprises cyclosporin A, Chemonic S-2®, and Clove oil.

Another embodiment comprises cyclosporin A, Chemonic S-10®, and Clove oil.

Another embodiment comprises cyclosporin A, Chemonic S-20®, and Clove oil.

Another embodiment comprises cyclosporin A, Colamulse FE®, and Clove oil.

Another embodiment comprises cyclosporin A, Cremophor A 20®, and Clove oil.

Another embodiment comprises cyclosporin A, Cremophor SA 2®, and Clove oil.

Another embodiment comprises cyclosporin A, Dehydol 100®, and Clove oil.

Another embodiment comprises cyclosporin A, Dehydol O-4®, and Clove oil.

Another embodiment comprises cyclosporin A, Delonic C-18®, and Clove oil.

Another embodiment comprises cyclosporin A, DeSonic 6T®, and Clove oil.

Another embodiment comprises cyclosporin A, DeSonic 9D®, and Clove oil.

Another embodiment comprises cyclosporin A, DeSonic 9T®, and Clove oil.

Another embodiment comprises cyclosporin A, DeSonic 12D®, and Clove oil.

Another embodiment comprises cyclosporin A, DeSonic 12T®, and Clove oil.

Another embodiment comprises cyclosporin A, DeSonic 15T®, and Clove oil.

Another embodiment comprises cyclosporin A, DeSonic TDA-9®, and Clove oil.

Another embodiment comprises cyclosporin A, DeThox GLG-7®, and Clove oil.

Another embodiment comprises cyclosporin A, DeThox GLG-26®, and Clove oil.

Another embodiment comprises cyclosporin A, DeThox LA-4®, and Clove oil.

Another embodiment comprises cyclosporin A, DeThox LA-23®, and Clove oil.

Another embodiment comprises cyclosporin A, DeThox SA-80®, and Clove oil.

Another embodiment comprises cyclosporin A, Disponil O5®, and Clove oil.

Another embodiment comprises cyclosporin A, Eccoterge EO-41B®, and Clove oil.

Another embodiment comprises cyclosporin A, Empilan KA2.5/90®, and Clove oil.

Another embodiment comprises cyclosporin A, Empilan KA5/90®, and Clove oil.

Another embodiment comprises cyclosporin A, Empilan KM-20®, and Clove oil.

Another embodiment comprises cyclosporin A, Empilan KM-50®, and Clove oil.

Another embodiment comprises cyclosporin A, Empilan L-23®, and Clove oil.

Another embodiment comprises cyclosporin A, Ethylan 25-3®, and Clove oil.

Another embodiment comprises cyclosporin A, Ethylan 1204®, and Clove oil.

Another embodiment comprises cyclosporin A, Ethylan DA-4®, and Clove oil.

Another embodiment comprises cyclosporin A, Ethylan LA-230®, and Clove oil.

Another embodiment comprises cyclosporin A, Ethylan SN®, and Clove oil.

Another embodiment comprises cyclosporin A, Ethylan TD-60®, and Clove oil.

Another embodiment comprises cyclosporin A, Ethylan TD-100®, and Clove oil.

Another embodiment comprises cyclosporin A, Ethylan TD-1407®, and Clove oil.

Another embodiment comprises cyclosporin A, Eumulgin B1®, and Clove oil.

Another embodiment comprises cyclosporin A, Eumulgin B2®, and Clove oil.

Another embodiment comprises cyclosporin A, Eumulgin B3®, and Clove oil.

Another embodiment comprises cyclosporin A, Eumulgin O-10®, and Clove oil.

Another embodiment comprises cyclosporin A, Flo Mo 80/20®, and Clove oil.

Another embodiment comprises cyclosporin A, Flo Mo Low Foam®, and Clove oil.

Another embodiment comprises cyclosporin A, Forlan C-24®, and Clove oil.

Another embodiment comprises cyclosporin A, Genapol 1454®, and Clove oil.

Another embodiment comprises cyclosporin A, Genapol BA-020®, and Clove oil.

Another embodiment comprises cyclosporin A, Genapol BA-040®, and Clove oil.

Another embodiment comprises cyclosporin A, Genapol C-100®, and Clove oil.

Another embodiment comprises cyclosporin A, Genapol DA 060®, and Clove oil.

Another embodiment comprises cyclosporin A, Genapol HS 020®, and Clove oil.

Another embodiment comprises cyclosporin A, Genapol HS 200®, and Clove oil.

Another embodiment comprises cyclosporin A, Genapol ID-040®, and Clove oil.

Another embodiment comprises cyclosporin A, Genapol ID-060®, and Clove oil.

Another embodiment comprises cyclosporin A, Genapol ID-090®, and Clove oil.

Another embodiment comprises cyclosporin A, Genapol LA 010®, and Clove oil.

Another embodiment comprises cyclosporin A, Genapol LA 020®, and Clove oil.

Another embodiment comprises cyclosporin A, Genapol LA 030®, and Clove oil.

Another embodiment comprises cyclosporin A, Genapol LA 040®, and Clove oil.

Another embodiment comprises cyclosporin A, Genapol LA 050®, and Clove oil.

Another embodiment comprises cyclosporin A, Genapol LA 060®, and Clove oil.

Another embodiment comprises cyclosporin A, Genapol LA 070®, and Clove oil.

Another embodiment comprises cyclosporin A, Genapol LA 070S®, and Clove oil.

Another embodiment comprises cyclosporin A, Genapol LA 230®, and Clove oil.

Another embodiment comprises cyclosporin A, Genapol O 020®, and Clove oil.

Another embodiment comprises cyclosporin A, Genapol O 050®, and Clove oil.

Another embodiment comprises cyclosporin A, Genapol O 100®, and Clove oil.

Another embodiment comprises cyclosporin A, Genapol O 200®, and Clove oil.

Another embodiment comprises cyclosporin A, Genapol SA 030®, and Clove oil.

Another embodiment comprises cyclosporin A, Genapol SA 120®, and Clove oil.

Another embodiment comprises cyclosporin A, Genapol T-020®, and Clove oil.

Another embodiment comprises cyclosporin A, Genapol UD-030®, and Clove oil.

Another embodiment comprises cyclosporin A, Genapol UD-050®, and Clove oil.

Another embodiment comprises cyclosporin A, Genapol UD-070®, and Clove oil.

Another embodiment comprises cyclosporin A, Genapol UD-079®, and Clove oil.

Another embodiment comprises cyclosporin A, Genapol UD-080®, and Clove oil.

Another embodiment comprises cyclosporin A, Genapol UD-110®, and Clove oil.

Another embodiment comprises cyclosporin A, Genapol X 030®, and Clove oil.

Another embodiment comprises cyclosporin A, Genapol X 0500, and Clove oil.

Another embodiment comprises cyclosporin A, Genapol X 060®, and Clove oil.

Another embodiment comprises cyclosporin A, Genapol X 070®, and Clove oil.

Another embodiment comprises cyclosporin A, Genapol X 080®, and Clove oil.

Another embodiment comprises cyclosporin A, Genapol X 100®, and Clove oil.

Another embodiment comprises cyclosporin A, Genapol X 159®, and Clove oil.

Another embodiment comprises cyclosporin A, Generol 122 E5®, and Clove oil.

Another embodiment comprises cyclosporin A, Generol 122 E25®, and Clove oil.

Another embodiment comprises cyclosporin A, Hostacerin T-3®, and Clove oil.

Another embodiment comprises cyclosporin A, Iconol LF 110®, and Clove oil.

Another embodiment comprises cyclosporin A, Incropol CS-20®, and Clove oil.

Another embodiment comprises cyclosporin A, Lexemul CS-20®, and Clove oil.

Another embodiment comprises cyclosporin A, Liponic EG-1®, and Clove oil.

Another embodiment comprises cyclosporin A, Lipowax D®, and Clove oil.

Another embodiment comprises cyclosporin A, Lipowax G®, and Clove oil.

Another embodiment comprises cyclosporin A, Lipowax NI®, and Clove oil.

Another embodiment comprises cyclosporin A, Lipowax P®, and Clove oil.

Another embodiment comprises cyclosporin A, Lipowax P-31®, and Clove oil.

Another embodiment comprises cyclosporin A, Lipowax PR®, and Clove oil.

Another embodiment comprises cyclosporin A, Lumulse CS-20®, and Clove oil.

Another embodiment comprises cyclosporin A, Macol CSA-20®, and Clove oil.

Another embodiment comprises cyclosporin A, Marlox B 24/50®, and Clove oil.

Another embodiment comprises cyclosporin A, Mazawet 77®, and Clove oil.

Another embodiment comprises cyclosporin A, Norfox 1713®, and Clove oil.

Another embodiment comprises cyclosporin A, Norfox 2579®, and Clove oil.

Another embodiment comprises cyclosporin A, Norfox Lo Foam®, and Clove oil.

Another embodiment comprises cyclosporin A, Promulgen D®, and Clove oil.

Another embodiment comprises cyclosporin A, Promulgen G®, and Clove oil.

Another embodiment comprises cyclosporin A, Renex 30®, and Clove oil.

Another embodiment comprises cyclosporin A, Renex 36®, and Clove oil.

Another embodiment comprises cyclosporin A, Rhodasurf A 24®, and Clove oil.

Another embodiment comprises cyclosporin A, Rhodasurf AAE-10®, and Clove oil.

Another embodiment comprises cyclosporin A, Rhodasurf BEH-25®, and Clove oil.

Another embodiment comprises cyclosporin A, Rhodasurf BEH-40®, and Clove oil.

Another embodiment comprises cyclosporin A, Rhodasurf DA 530®, and Clove oil.

Another embodiment comprises cyclosporin A, Rhodasurf DA 630®, and Clove oil.

Another embodiment comprises cyclosporin A, Rhodasurf DA 639®, and Clove oil.

Another embodiment comprises cyclosporin A, Rhodasurf LAN-23®, and Clove oil.

Another embodiment comprises cyclosporin A, Rhodasurf ON-870®, and Clove oil.

Another embodiment comprises cyclosporin A, Rhodasurf ON-877®, and Clove oil.

Another embodiment comprises cyclosporin A, Rhodasurf TB-970 FLK®, and Clove oil.

Another embodiment comprises cyclosporin A, Ritacet-20®, and Clove oil.

Another embodiment comprises cyclosporin A, Ritachol 1000®, and Clove oil.

Another embodiment comprises cyclosporin A, Ritachol 2000®, and Clove oil.

Another embodiment comprises cyclosporin A, Ritachol 5000®, and Clove oil.

Another embodiment comprises cyclosporin A, Ritox 35®, and Clove oil.

Another embodiment comprises cyclosporin A, Surfonic DA-4®, and Clove oil.

Another embodiment comprises cyclosporin A, Suqonic DA-6®, and Clove oil.

Another embodiment comprises cyclosporin A, Surfonic L46-7®, and Clove oil.

Another embodiment comprises cyclosporin A, Surfonic POA®, and Clove oil.

Another embodiment comprises cyclosporin A, Synthrapol KB®, and Clove oil.

Another embodiment comprises cyclosporin A, Teginacid®, and Clove oil.

Another embodiment comprises cyclosporin A, Teginacid C®, and Clove oil.

Another embodiment comprises cyclosporin A, Tegotens EC 11®, and Clove oil.

Another embodiment comprises cyclosporin A, Tinegal NA®, and Clove oil.

Another embodiment comprises cyclosporin A, Tomadol 400®, and Clove oil.

Another embodiment comprises cyclosporin A, Tomadol 600®, and Clove oil.

Another embodiment comprises cyclosporin A, Tomadol 900®, and Clove oil.

Another embodiment comprises cyclosporin A, Uniperol O®, and Clove oil.

Another embodiment comprises cyclosporin A, Witconol SN Series®, and Clove oil.

Another embodiment comprises cyclosporin A, an Ethoxylated Alkylphenol, and Clove oil.

Another embodiment comprises cyclosporin A, Antarox LF-222®, and Clove oil.

Another embodiment comprises cyclosporin A, Atlox 775®, and Clove oil.

Another embodiment comprises cyclosporin A, Caloxylate N-9®, and Clove oil.

Another embodiment comprises cyclosporin A, Canasol NF-1000®, and Clove oil.

Another embodiment comprises cyclosporin A, Canasol NF-3000®, and Clove oil.

Another embodiment comprises cyclosporin A, Canasol NF-3070®, and Clove oil.

Another embodiment comprises cyclosporin A, Canasol OF 1670®, and Clove oil.

Another embodiment comprises cyclosporin A, Canasol OF 2570®, and Clove oil.

Another embodiment comprises cyclosporin A, Canasol OF 4070®, and Clove oil.

Another embodiment comprises cyclosporin A, Chemax DNP-8®, and Clove oil.

Another embodiment comprises cyclosporin A, Chemax DNP-18®, and Clove oil.

Another embodiment comprises cyclosporin A, Chemax DNP-150/50®, and Clove oil.

Another embodiment comprises cyclosporin A, DeSonic 1.5N®, and Clove oil.

Another embodiment comprises cyclosporin A, DeSonic 4N®, and Clove oil.

Another embodiment comprises cyclosporin A, DeSonic 5N®, and Clove oil.

Another embodiment comprises cyclosporin A, DeSonic 6D®, and Clove oil.

Another embodiment comprises cyclosporin A, DeSonic 6N®, and Clove oil.

Another embodiment comprises cyclosporin A, DeSonic 7N®, and Clove oil.

Another embodiment comprises cyclosporin A, DeSonic 9N®, and Clove oil.

Another embodiment comprises cyclosporin A, DeSonic 10D®, and Clove oil.

Another embodiment comprises cyclosporin A, DeSonic 11N®, and Clove oil.

Another embodiment comprises cyclosporin A, DeSonic 12N®, and Clove oil.

Another embodiment comprises cyclosporin A, DeSonic 15N®, and Clove oil.

Another embodiment comprises cyclosporin A, DeSonic 20N®, and Clove oil.

Another embodiment comprises cyclosporin A, Eccoscour RC®, and Clove oil.

Another embodiment comprises cyclosporin A, Eccoterge EO-100®, and Clove oil.

Another embodiment comprises cyclosporin A, Emulsifier 632/90%®, and Clove oil.

Another embodiment comprises cyclosporin A, Geronol AG-821®, and Clove oil.

Another embodiment comprises cyclosporin A, Gradonic N-95®, and Clove oil.

Another embodiment comprises cyclosporin A, Hetoxide NP-4®, and Clove oil.

Another embodiment comprises cyclosporin A, Hetoxide NP-30®, and Clove oil.

Another embodiment comprises cyclosporin A, Hostapal N-10®, and Clove oil.

Another embodiment comprises cyclosporin A, Hostapal N-110®, and Clove oil.

Another embodiment comprises cyclosporin A, Igepal CTA-639W®, and Clove oil.

Another embodiment comprises cyclosporin A, Igepal DAP-9®, and Clove oil.

Another embodiment comprises cyclosporin A, Igepal OD-410®, and Clove oil.

Another embodiment comprises cyclosporin A, Igepal SS-837®, and Clove oil.

Another embodiment comprises cyclosporin A, Lipocol NP-9 USP®, and Clove oil.

Another embodiment comprises cyclosporin A, Macol DNP-10®, and Clove oil.

Another embodiment comprises cyclosporin A, Marlophen NP 5®, and Clove oil.

Another embodiment comprises cyclosporin A, Marlophen P 1®, and Clove oil.

Another embodiment comprises cyclosporin A, Surfonic NB®, and Clove oil.

Another embodiment comprises cyclosporin A, Suronic OPB-307®, and Clove oil.

Another embodiment comprises cyclosporin A, Surfonic OPB-407®, and Clove oil.

Another embodiment comprises cyclosporin A, Syn Fac 334®, and Clove oil.

Another embodiment comprises cyclosporin A, Syn Fac 8216®, and Clove oil.

Another embodiment comprises cyclosporin A, Triton N-57®, and Clove oil.

Another embodiment comprises cyclosporin A, Trycol 6956®, and Clove oil.

Another embodiment comprises cyclosporin A, Trycol 6961®, and Clove oil.

Another embodiment comprises cyclosporin A, Trycol 6964®, and Clove oil.

Another embodiment comprises cyclosporin A, Trycol 6969®, and Clove oil.

Another embodiment comprises cyclosporin A, Trycol 6974®, and Clove oil.

Another embodiment comprises cyclosporin A, Witbreak DRB-127®, and Clove oil.

Another embodiment comprises cyclosporin A, Witbreak DRB-127®, and Clove oil.

Another embodiment comprises cyclosporin A, Witconol NP Series®, and Clove oil.

Another embodiment comprises cyclosporin A, an Ethoxylated Aryl Phenol, and Clove oil.

Another embodiment comprises cyclosporin A, Soprophor BSU®, and Clove oil.

Another embodiment comprises cyclosporin A, Soprophor CY/8®, and Clove oil.

Another embodiment comprises cyclosporin A, Soprophor S/25®, and Clove oil.

Another embodiment comprises cyclosporin A, Witconol NIO®, and Clove oil.

Another embodiment comprises cyclosporin A, Witconol NIW®, and Clove oil.

Another embodiment comprises cyclosporin A, Witconol S-100®, and Clove oil.

Another embodiment comprises cyclosporin A, an Ethoxylated Fatty Acid, and Clove oil.

Another embodiment comprises cyclosporin A, Aldo PGHMS®, and Clove oil.

Another embodiment comprises cyclosporin A, Alkamuls TO-15/HR®, and Clove oil.

Another embodiment comprises cyclosporin A, Armotan AL-69-66®, and Clove oil.

Another embodiment comprises cyclosporin A, Cerasynt 840®, and Clove oil.

Another embodiment comprises cyclosporin A, Cerasynt 945®, and Clove oil.

Another embodiment comprises cyclosporin A, Crystal Inhibitor No. 5®, and Clove oil.

Another embodiment comprises cyclosporin A, DeThox Acid L-9®, and Clove oil.

Another embodiment comprises cyclosporin A, DeThox Acid S-8®, and Clove oil.

Another embodiment comprises cyclosporin A, Ethofat 242/25®, and Clove oil.

Another embodiment comprises cyclosporin A, Hydropalat 65®, and Clove oil.

Another embodiment comprises cyclosporin A, Lipo EGMS®, and Clove oil.

Another embodiment comprises cyclosporin A, Lipopeg 2 DL®, and Clove oil.

Another embodiment comprises cyclosporin A, Lipopeg 4 DL®, and Clove oil.

Another embodiment comprises cyclosporin A, Lipopeg 4-L®, and Clove oil.

Another embodiment comprises cyclosporin A, Lipopeg 39-S®, and Clove oil.

Another embodiment comprises cyclosporin A, Lipopeg 4-S®, and Clove oil.

Another embodiment comprises cyclosporin A, Lipopeg 10-S®, and Clove oil.

Another embodiment comprises cyclosporin A, Lipopeg 100-S®, and Clove oil.

Another embodiment comprises cyclosporin A, Lipopeg 6000 DS®, and Clove oil.

Another embodiment comprises cyclosporin A, Lumulse 40-L®, and Clove oil.

Another embodiment comprises cyclosporin A, Lumulse 40-S®, and Clove oil.

Another embodiment comprises cyclosporin A, Lumulse 42-L®, and Clove oil.

Another embodiment comprises cyclosporin A, Lumulse 42-S®, and Clove oil.

Another embodiment comprises cyclosporin A, Lumulse 100-S®, and Clove oil.

Another embodiment comprises cyclosporin A, Lumulse 602-S®, and Clove oil.

Another embodiment comprises cyclosporin A, Magrabar PGE-20-0®, and Clove oil.

Another embodiment comprises cyclosporin A, Magrabar PGE-20L®, and Clove oil.

Another embodiment comprises cyclosporin A, Magrabar PGE-20T®, and Clove oil.

Another embodiment comprises cyclosporin A, Magrabar PGE-22-0®, and Clove oil.

Another embodiment comprises cyclosporin A, Magrabar PGE-22L®, and Clove oil.

Another embodiment comprises cyclosporin A, Magrabar PGE-22T®, and Clove oil.

Another embodiment comprises cyclosporin A, Magrabar PGE-40-0®, and Clove oil.

Another embodiment comprises cyclosporin A, Magrabar PGE-40L®, and Clove oil.

Another embodiment comprises cyclosporin A, Magrabar PGE-40T®, and Clove oil.

Another embodiment comprises cyclosporin A, Magrabar PGE-42-0®, and Clove oil.

Another embodiment comprises cyclosporin A, Magrabar PGE-42L®, and Clove oil.

Another embodiment comprises cyclosporin A, Magrabar PGE-42T®, and Clove oil.

Another embodiment comprises cyclosporin A, Magrabar PGE-60-0®, and Clove oil.

Another embodiment comprises cyclosporin A, Magrabar PGE-60L®, and Clove oil.

Another embodiment comprises cyclosporin A, Magrabar PGE-60T®, and Clove oil.

Another embodiment comprises cyclosporin A, Magrabar PGE-62-0®, and Clove oil.

Another embodiment comprises cyclosporin A, Magrabar PGE-62L®, and Clove oil.

Another embodiment comprises cyclosporin A, Magrabar PGE-62T®, and Clove oil.

Another embodiment comprises cyclosporin A, Mapeg S-40K®, and Clove oil.

Another embodiment comprises cyclosporin A, Marlowet OTS®, and Clove oil.

Another embodiment comprises cyclosporin A, Naturechem PGR®, and Clove oil.

Another embodiment comprises cyclosporin A, PG No. 4®, and Clove oil.

Another embodiment comprises cyclosporin A, Renex 20®, and Clove oil.

Another embodiment comprises cyclosporin A, Ritox 52®, and Clove oil.

Another embodiment comprises cyclosporin A, Ritox 53®, and Clove oil.

Another embodiment comprises cyclosporin A, Ritox 59®, and Clove oil.

Another embodiment comprises cyclosporin A, Surfax 8916/A®, and Clove oil.

Another embodiment comprises cyclosporin A, Tego Acid S 40 P®, and Clove oil.

Another embodiment comprises cyclosporin A, Tego Acid S 100 P®, and Clove oil.

Another embodiment comprises cyclosporin A, Tween 20®, and Clove oil.

Another embodiment comprises cyclosporin A, Volpo 131®, and Clove oil.

Another embodiment comprises cyclosporin A, an Ethoxylated Fatty Ester or Oil, and Clove oil.

Another embodiment comprises cyclosporin A, Acconon 6-C10®, and Clove oil.

Another embodiment comprises cyclosporin A, Acconon CC-6®, and Clove oil.

Another embodiment comprises cyclosporin A, Acconon CO-7®, and Clove oil.

Another embodiment comprises cyclosporin A, Aldosperse 40/60 FG®, and Clove oil.

Another embodiment comprises cyclosporin A, Aldosperse ML-23®, and Clove oil.

Another embodiment comprises cyclosporin A, Aldosperse MS-20 FG®, and Clove oil.

Another embodiment comprises cyclosporin A, Alkamuls EL-620®, and Clove oil.

Another embodiment comprises cyclosporin A, Alkamuls EL-719®, and Clove oil.

Another embodiment comprises cyclosporin A, Alkamuls EL-985®, and Clove oil.

Another embodiment comprises cyclosporin A, Arlatone G®, and Clove oil.

Another embodiment comprises cyclosporin A, Arlatone T®, and Clove oil.

Another embodiment comprises cyclosporin A, Atlas G-1045A®, and Clove oil.

Another embodiment comprises cyclosporin A, Atlas G-1086®, and Clove oil.

Another embodiment comprises cyclosporin A, Atlas G-1087®, and Clove oil.

Another embodiment comprises cyclosporin A, Atlas G-1089®, and Clove oil.

Another embodiment comprises cyclosporin A, Atlas G-1096®, and Clove oil.

Another embodiment comprises cyclosporin A, Atlas G-1292®, and Clove oil.

Another embodiment comprises cyclosporin A, Atlas G-1293®, and Clove oil.

Another embodiment comprises cyclosporin A, Atlas G-1300®, and Clove oil.

Another embodiment comprises cyclosporin A, Atlas G-7076®, and Clove oil.

Another embodiment comprises cyclosporin A, Capmul EMG®, and Clove oil.

Another embodiment comprises cyclosporin A, Chemonic CO-40®, and Clove oil.

Another embodiment comprises cyclosporin A, Chemonic LI-3®, and Clove oil.

Another embodiment comprises cyclosporin A, Chemonic LI-7®, and Clove oil.

Another embodiment comprises cyclosporin A, Cirrasol GM®, and Clove oil.

Another embodiment comprises cyclosporin A, Cremophor CO 40®, and Clove oil.

Another embodiment comprises cyclosporin A, Cremophor CO 410®, and Clove oil.

Another embodiment comprises cyclosporin A, Cremophor EL®, and Clove oil.

Another embodiment comprises cyclosporin A, Cremophor GC7®, and Clove oil.

Another embodiment comprises cyclosporin A, Cremophor RH-40®, and Clove oil.

Another embodiment comprises cyclosporin A, Crovol A-40®, and Clove oil.

Another embodiment comprises cyclosporin A, Crovol A-70®, and Clove oil.

Another embodiment comprises cyclosporin A, Crovol M-70®, and Clove oil.

Another embodiment comprises cyclosporin A, Crovol PK-700, and Clove oil.

Another embodiment comprises cyclosporin A, Cutina E-24®, and Clove oil.

Another embodiment comprises cyclosporin A, Dacospin 12-R®, and Clove oil.

Another embodiment comprises cyclosporin A, Dehymuls HRE-7®, and Clove oil.

Another embodiment comprises cyclosporin A, DeSonic 30C®, and Clove oil.

Another embodiment comprises cyclosporin A, DeSonic 36C®, and Clove oil.

Another embodiment comprises cyclosporin A, DeSonic 40C®, and Clove oil.

Another embodiment comprises cyclosporin A, Durfax 60®, and Clove oil.

Another embodiment comprises cyclosporin A, Durfax 65®, and Clove oil.

Another embodiment comprises cyclosporin A, Durfax 80®, and Clove oil.

Another embodiment comprises cyclosporin A, Durfax EOM®, and Clove oil.

Another embodiment comprises cyclosporin A, Eccoterge NF-2®, and Clove oil.

Another embodiment comprises cyclosporin A, Emulpon CO-360®, and Clove oil.

Another embodiment comprises cyclosporin A, Emulpon CO-550®, and Clove oil.

Another embodiment comprises cyclosporin A, Emulsogen EL®, and Clove oil.

Another embodiment comprises cyclosporin A, Emulsogen HCO 040®, and Clove oil.

Another embodiment comprises cyclosporin A, Emulsogen HCO 060®, and Clove oil.

Another embodiment comprises cyclosporin A, Emulsynt 1055®, and Clove oil.

Another embodiment comprises cyclosporin A, Ethox 3095®, and Clove oil.

Another embodiment comprises cyclosporin A, Eumulgin RO-40®, and Clove oil.

Another embodiment comprises cyclosporin A, Genapol G-260®, and Clove oil.

Another embodiment comprises cyclosporin A, Glycosperse L-20®, and Clove oil.

Another embodiment comprises cyclosporin A, Glycosperse O-5®, and Clove oil.

Another embodiment comprises cyclosporin A, Glycosperse O-20®, and Clove oil.

Another embodiment comprises cyclosporin A, Glycosperse O-20 FG®, and Clove oil.

Another embodiment comprises cyclosporin A, Glycosperse S-20®, and Clove oil.

Another embodiment comprises cyclosporin A, Glycosperse S-20 FG®, and Clove oil.

Another embodiment comprises cyclosporin A, Glycosperse TS-20®, and Clove oil.

Another embodiment comprises cyclosporin A, Glycosperse TS-20 FG®, and Clove oil.

Another embodiment comprises cyclosporin A, Hetan SL®, and Clove oil.

Another embodiment comprises cyclosporin A, Hetan SO®, and Clove oil.

Another embodiment comprises cyclosporin A, Hetan SS®, and Clove oil.

Another embodiment comprises cyclosporin A, Hetoxide C-2®, and Clove oil.

Another embodiment comprises cyclosporin A, Hetoxide C-9®, and Clove oil.

Another embodiment comprises cyclosporin A, Hetoxide C-15®, and Clove oil.

Another embodiment comprises cyclosporin A, Hetoxide C-25®, and Clove oil.

Another embodiment comprises cyclosporin A, Hetoxide C-40®, and Clove oil.

Another embodiment comprises cyclosporin A, Hetoxide C-200®, and Clove oil.

Another embodiment comprises cyclosporin A, Hetoxide C-200-50%®, and Clove oil.

Another embodiment comprises cyclosporin A, Hetoxide GC-30®, and Clove oil.

Another embodiment comprises cyclosporin A, Hetoxide HC-60®, and Clove oil.

Another embodiment comprises cyclosporin A, Ice No. 2®, and Clove oil.

Another embodiment comprises cyclosporin A, Incrocas 30/40®, and Clove oil.

Another embodiment comprises cyclosporin A, Lexol EC®, and Clove oil.

Another embodiment comprises cyclosporin A, Lexol EO®, and Clove oil.

Another embodiment comprises cyclosporin A, Lipocol HCO-40®, and Clove oil.

Another embodiment comprises cyclosporin A, Lipocol HCO-60®, and Clove oil.

Another embodiment comprises cyclosporin A, Lipocol O-3 Special®, and Clove oil.

Another embodiment comprises cyclosporin A, Lipopeg 2-L®, and Clove oil.

Another embodiment comprises cyclosporin A, Lipopeg 4-DO®, and Clove oil.

Another embodiment comprises cyclosporin A, Lipopeg 4-DS®, and Clove oil.

Another embodiment comprises cyclosporin A, Lipovol GTB®, and Clove oil.

Another embodiment comprises cyclosporin A, Lonzest SML-20®, and Clove oil.

Another embodiment comprises cyclosporin A, Lonzest SMO-20®, and Clove oil.

Another embodiment comprises cyclosporin A, Lonzest SMS-20®, and Clove oil.

Another embodiment comprises cyclosporin A, Lonzest STO-20®, and Clove oil.

Another embodiment comprises cyclosporin A, Lonzest STS-20®, and Clove oil.

Another embodiment comprises cyclosporin A, Lumulse GR-40®, and Clove oil.

Another embodiment comprises cyclosporin A, Lumulse GRH-40®, and Clove oil.

Another embodiment comprises cyclosporin A, Lumulse POE (7) GML®, and Clove oil.

Another embodiment comprises cyclosporin A, Lumulse POE (12) Glyc®, and Clove oil.

Another embodiment comprises cyclosporin A, Lumulse POE (40) MS KP®, and Clove oil.

Another embodiment comprises cyclosporin A, Marlowet 4750®, and Clove oil.

Another embodiment comprises cyclosporin A, Marlowet LVS®, and Clove oil.

Another embodiment comprises cyclosporin A, Marlowet R 11®, and Clove oil.

Another embodiment comprises cyclosporin A, Marlowet R 40®, and Clove oil.

Another embodiment comprises cyclosporin A, Mazol 80 MGK®, and Clove oil.

Another embodiment comprises cyclosporin A, Nonionic Emulsifier T-9®, and Clove oil.

Another embodiment comprises cyclosporin A, Oronal LCG®, and Clove oil.

Another embodiment comprises cyclosporin A, Polyderm PPI-CO-200®, and Clove oil.

Another embodiment comprises cyclosporin A, Polyderm PPI-CO-40®, and Clove oil.

Another embodiment comprises cyclosporin A, Rewoderm LI 520-70®, and Clove oil.

Another embodiment comprises cyclosporin A, Ritapeg 150 DS®, and Clove oil.

Another embodiment comprises cyclosporin A, Softigen 767®, and Clove oil.

Another embodiment comprises cyclosporin A, Surtactol 318®, and Clove oil.

Another embodiment comprises cyclosporin A, Surtactol 365®, and Clove oil.

Another embodiment comprises cyclosporin A, Syn Lube 107®, and Clove oil.

Another embodiment comprises cyclosporin A, Syn Lube 728®, and Clove oil.

Another embodiment comprises cyclosporin A, Syn Lube 1632H®, and Clove oil.

Another embodiment comprises cyclosporin A, Syn Lube 6277-A®, and Clove oil.

Another embodiment comprises cyclosporin A, T-Det C-20®, and Clove oil.

Another embodiment comprises cyclosporin A, T-Det C-40®, and Clove oil.

Another embodiment comprises cyclosporin A, Tally 100 Plus®, and Clove oil.

Another embodiment comprises cyclosporin A, Uniperol EL®, and Clove oil.

Another embodiment comprises cyclosporin A, a Fatty Ester, and Clove oil.

Another embodiment comprises cyclosporin A, Actralube-Syn 147®, and Clove oil.

Another embodiment comprises cyclosporin A, Atlas G-1556®, and Clove oil.

Another embodiment comprises cyclosporin A, Atlas G-1564®, and Clove oil.

Another embodiment comprises cyclosporin A, Atlasol Base Oil S®, and Clove oil.

Another embodiment comprises cyclosporin A, Base ML®, and Clove oil.

Another embodiment comprises cyclosporin A, Base MT®, and Clove oil.

Another embodiment comprises cyclosporin A, Cerasynt 303®, and Clove oil.

Another embodiment comprises cyclosporin A, Dermol 1012®, and Clove oil.

Another embodiment comprises cyclosporin A, Kemester 4000®, and Clove oil.

Another embodiment comprises cyclosporin A, Lactipol S®, and Clove oil.

Another embodiment comprises cyclosporin A, Magrabar PGO®, and Clove oil.

Another embodiment comprises cyclosporin A, Mayco Base BFO®, and Clove oil.

Another embodiment comprises cyclosporin A, Methyl Linoleate®, and Clove oil.

Another embodiment comprises cyclosporin A, Pationic 122A®, and Clove oil.

Another embodiment comprises cyclosporin A, Pationic 138C®, and Clove oil.

Another embodiment comprises cyclosporin A, Pationic CSL®, and Clove oil.

Another embodiment comprises cyclosporin A, Pationic ISL®, and Clove oil.

Another embodiment comprises cyclosporin A, Pationic SBL®, and Clove oil.

Another embodiment comprises cyclosporin A, Pationic SSL®, and Clove oil.

Another embodiment comprises cyclosporin A, Ritasol®, and Clove oil.

Another embodiment comprises cyclosporin A, Tego Alkanol CS 20®, and Clove oil.

Another embodiment comprises cyclosporin A, Tego Alkanol L23 P®, and Clove oil.

Another embodiment comprises cyclosporin A, Tego Alkanol S2®, and Clove oil.

Another embodiment comprises cyclosporin A, Tego Alkanol S20 P®, and Clove oil.

Another embodiment comprises cyclosporin A, Triemulsifier 600 MS®, and Clove oil.

Another embodiment comprises cyclosporin A, a Glycerol Esters, and Clove oil.

Another embodiment comprises cyclosporin A, Agro #9 Wint SBO®, and Clove oil.

Another embodiment comprises cyclosporin A, Ahcovel Base 700®, and Clove oil.

Another embodiment comprises cyclosporin A, Aldo HMS FG®, and Clove oil.

Another embodiment comprises cyclosporin A, Aldo MLD®, and Clove oil.

Another embodiment comprises cyclosporin A, Aldo MLD FG®, and Clove oil.

Another embodiment comprises cyclosporin A, Aldo MO FG®, and Clove oil.

Another embodiment comprises cyclosporin A, Aldo MS®, and Clove oil.

Another embodiment comprises cyclosporin A, Aldo MS FG®, and Clove oil.

Another embodiment comprises cyclosporin A, Aldo MS LG FG®, and Clove oil.

Another embodiment comprises cyclosporin A, Aldo MSD®, and Clove oil.

Another embodiment comprises cyclosporin A, Aldo MSD FG®, and Clove oil.

Another embodiment comprises cyclosporin A, Aldosperse O-20 FG®, and Clove oil.

Another embodiment comprises cyclosporin A, Aldosperse TS-20 FG®, and Clove oil.

Another embodiment comprises cyclosporin A, Aldosperse TS-40 FG®, and Clove oil.

Another embodiment comprises cyclosporin A, Arlacel 165®, and Clove oil.

Another embodiment comprises cyclosporin A, Arlacel 186®, and Clove oil.

Another embodiment comprises cyclosporin A, Capmul GMO®, and Clove oil.

Another embodiment comprises cyclosporin A, Capmul GMS®, and Clove oil.

Another embodiment comprises cyclosporin A, Caprol 3GO®, and Clove oil.

Another embodiment comprises cyclosporin A, Caprol 3GVS®®, and Clove oil.

Another embodiment comprises cyclosporin A, Caprol 6G2S®, and Clove oil.

Another embodiment comprises cyclosporin A, Caprol 10G40®, and Clove oil.

Another embodiment comprises cyclosporin A, Caprol 10G100®, and Clove oil.

Another embodiment comprises cyclosporin A, Caprol ET®, and Clove oil.

Another embodiment comprises cyclosporin A, Caprol PGE860®, and Clove oil.

Another embodiment comprises cyclosporin A, Cerasynt 945®, and Clove oil.

Another embodiment comprises cyclosporin A, Cerasynt GMS®, and Clove oil.

Another embodiment comprises cyclosporin A, Cerasynt Q®, and Clove oil.

Another embodiment comprises cyclosporin A, Cerasynt SD®, and Clove oil.

Another embodiment comprises cyclosporin A, Cerasynt WM®, and Clove oil.

Another embodiment comprises cyclosporin A, Chemsperse 14®, and Clove oil.

Another embodiment comprises cyclosporin A, Cremophor GO-32®, and Clove oil.

Another embodiment comprises cyclosporin A, Cremophor GS11®, and Clove oil.

Another embodiment comprises cyclosporin A, Cremophor GS-32®, and Clove oil.

Another embodiment comprises cyclosporin A, Cutina KD-16®, and Clove oil.

Another embodiment comprises cyclosporin A, Dehymuls PGPH®, and Clove oil.

Another embodiment comprises cyclosporin A, Dermol DGDIS®, and Clove oil.

Another embodiment comprises cyclosporin A, Dermol DGMIS®, and Clove oil.

Another embodiment comprises cyclosporin A, Dermol G-76®, and Clove oil.

Another embodiment comprises cyclosporin A, Dermol G-7DI®, and Clove oil.

Another embodiment comprises cyclosporin A, Dermol NGDI®, and Clove oil.

Another embodiment comprises cyclosporin A, Dermolan GLH®, and Clove oil.

Another embodiment comprises cyclosporin A, Drewmulse GMG®, and Clove oil.

Another embodiment comprises cyclosporin A, Drewpol 3-5-M®, and Clove oil.

Another embodiment comprises cyclosporin A, Durlac 100 W®, and Clove oil.

Another embodiment comprises cyclosporin A, Dur-Lo®, and Clove oil.

Another embodiment comprises cyclosporin A, Dynasan 118®, and Clove oil.

Another embodiment comprises cyclosporin A, EC-25®, and Clove oil.

Another embodiment comprises cyclosporin A, EM 40®, and Clove oil.

Another embodiment comprises cyclosporin A, Emerest 2400®, and Clove oil.

Another embodiment comprises cyclosporin A, Emerest 2452®, and Clove oil.

Another embodiment comprises cyclosporin A, Empilan G-26®, and Clove oil.

Another embodiment comprises cyclosporin A, Genapol TSM®, and Clove oil.

Another embodiment comprises cyclosporin A, Hostacerin DGI®, and Clove oil.

Another embodiment comprises cyclosporin A, Hostacerin DGL®, and Clove oil.

Another embodiment comprises cyclosporin A, Hostacerin DGMS®, and Clove oil.

Another embodiment comprises cyclosporin A, Hostacerin DGSB®, and Clove oil.

Another embodiment comprises cyclosporin A, Ice No. 2®, and Clove oil.

Another embodiment comprises cyclosporin A, Imwitor 742®, and Clove oil.

Another embodiment comprises cyclosporin A, Imwitor 780 K®, and Clove oil.

Another embodiment comprises cyclosporin A, Imwitor 960 Flakes®, and Clove oil.

Another embodiment comprises cyclosporin A, Isolan GI 34®, and Clove oil.

Another embodiment comprises cyclosporin A, Isolan GO 33®, and Clove oil.

Another embodiment comprises cyclosporin A, Kemester 1000®, and Clove oil.

Another embodiment comprises cyclosporin A, Kemester 2000®, and Clove oil.

Another embodiment comprises cyclosporin A, Kemester 2000®, and Clove oil.

Another embodiment comprises cyclosporin A, Kemester 6000SE®, and Clove oil.

Another embodiment comprises cyclosporin A, Lamecreme DGE 18®, and Clove oil.

Another embodiment comprises cyclosporin A, Lexemul 515®, and Clove oil.

Another embodiment comprises cyclosporin A, Lexemul 561®, and Clove oil.

Another embodiment comprises cyclosporin A, Lexemul AR®, and Clove oil.

Another embodiment comprises cyclosporin A, Lexemul AS®, and Clove oil.

Another embodiment comprises cyclosporin A, Lexemul GDL®, and Clove oil.

Another embodiment comprises cyclosporin A, Lexemul T®, and Clove oil.

Another embodiment comprises cyclosporin A, Lipomulse 165®, and Clove oil.

Another embodiment comprises cyclosporin A, Lumulse GML K®, and Clove oil.

Another embodiment comprises cyclosporin A, Lumulse GMO K®, and Clove oil.

Another embodiment comprises cyclosporin A, Lumulse GMR K®, and Clove oil.

Another embodiment comprises cyclosporin A, Lumulse GMT K®, and Clove oil.

Another embodiment comprises cyclosporin A, Magrabar GMC®, and Clove oil.

Another embodiment comprises cyclosporin A, Magrabar GMO-CK®, and Clove oil.

Another embodiment comprises cyclosporin A, Magrabar MDG-5050®, and Clove oil.

Another embodiment comprises cyclosporin A, Magrabar PGO-315®, and Clove oil.

Another embodiment comprises cyclosporin A, Magrabar PGO-1010®, and Clove oil.

Another embodiment comprises cyclosporin A, Mazol 300K®, and Clove oil.

Another embodiment comprises cyclosporin A, Mazol GMO-K®, and Clove oil.

Another embodiment comprises cyclosporin A, Mazol GMS-K®, and Clove oil.

Another embodiment comprises cyclosporin A, Mazol PGO31-K®, and Clove oil.

Another embodiment comprises cyclosporin A, Miglyol 812®, and Clove oil.

Another embodiment comprises cyclosporin A, Nonox 165C®, and Clove oil.

Another embodiment comprises cyclosporin A, Schercemol GMIS®, and Clove oil.

Another embodiment comprises cyclosporin A, Tegin®, and Clove oil.

Another embodiment comprises cyclosporin A, Tegin 4100 Pellets®, and Clove oil.

Another embodiment comprises cyclosporin A, Tegin M Pellets®, and Clove oil.

Another embodiment comprises cyclosporin A, Tegin OV®, and Clove oil.

Another embodiment comprises cyclosporin A, Teginacid H®, and Clove oil.

Another embodiment comprises cyclosporin A, Tego Cosmo P813®, and Clove oil.

Another embodiment comprises cyclosporin A, Wickenol 535®, and Clove oil.

Another embodiment comprises cyclosporin A, Witconol 14®, and Clove oil.

Another embodiment comprises cyclosporin A, Witconol 14®, and Clove oil.

Another embodiment comprises cyclosporin A, Witconol 14F®, and Clove oil.

Another embodiment comprises cyclosporin A, Witconol 18L®, and Clove oil.

Another embodiment comprises cyclosporin A, Witconol GOT®, and Clove oil.

Another embodiment comprises cyclosporin A, Witconol MST®, and Clove oil.

Another embodiment comprises cyclosporin A, Witconol RHT®, and Clove oil.

Another embodiment comprises cyclosporin A, a Glycol Ester, and Clove oil.

Another embodiment comprises cyclosporin A, Alkamuls 600 DO®, and Clove oil.

Another embodiment comprises cyclosporin A, Alkamuls SEG®, and Clove oil.

Another embodiment comprises cyclosporin A, Atlas EM-2®, and Clove oil.

Another embodiment comprises cyclosporin A, Cerasynt IP®, and Clove oil.

Another embodiment comprises cyclosporin A, Cerasynt M®, and Clove oil.

Another embodiment comprises cyclosporin A, Cerasynt MN®, and Clove oil.

Another embodiment comprises cyclosporin A, Cerasynt PA®, and Clove oil.

Another embodiment comprises cyclosporin A, Chemsperse EGDS®, and Clove oil.

Another embodiment comprises cyclosporin A, Chemsperse EGMS®, and Clove oil.

Another embodiment comprises cyclosporin A, Colonial Monolaurin®, and Clove oil.

Another embodiment comprises cyclosporin A, DeMuls SGE-95®, and Clove oil.

Another embodiment comprises cyclosporin A, Eccoterge 200®, and Clove oil.

Another embodiment comprises cyclosporin A, Emerest 2380®, and Clove oil.

Another embodiment comprises cyclosporin A, Ethox 2610®, and Clove oil.

Another embodiment comprises cyclosporin A, Ethox DO-9®, and Clove oil.

Another embodiment comprises cyclosporin A, Ethox DO-14®, and Clove oil.

Another embodiment comprises cyclosporin A, Ethox SO-9®, and Clove oil.

Another embodiment comprises cyclosporin A, Fizul MD-318®, and Clove oil.

Another embodiment comprises cyclosporin A, Genapol EGDS-VHP®, and Clove oil.

Another embodiment comprises cyclosporin A, Genapol TS Powder®, and Clove oil.

Another embodiment comprises cyclosporin A, Hostacerin WO®, and Clove oil.

Another embodiment comprises cyclosporin A, Inversol 140®, and Clove oil.

Another embodiment comprises cyclosporin A, Kemester 104®, and Clove oil.

Another embodiment comprises cyclosporin A, Kemester 205®, and Clove oil.

Another embodiment comprises cyclosporin A, Kemester 226®, and Clove oil.

Another embodiment comprises cyclosporin A, Kemester 5221 SE®, and Clove oil.

Another embodiment comprises cyclosporin A, Kemester EGDS®, and Clove oil.

Another embodiment comprises cyclosporin A, Lexemul EGDS®, and Clove oil.

Another embodiment comprises cyclosporin A, Lexemul EGMS®, and Clove oil.

Another embodiment comprises cyclosporin A, Lexemul P®, and Clove oil.

Another embodiment comprises cyclosporin A, Lipo DGLS®, and Clove oil.

Another embodiment comprises cyclosporin A, Lipo EGDS®, and Clove oil.

Another embodiment comprises cyclosporin A, Lipo PGMS®, and Clove oil.

Another embodiment comprises cyclosporin A, Liposorb S-4®, and Clove oil.

Another embodiment comprises cyclosporin A, Liposorb TO-20®, and Clove oil.

Another embodiment comprises cyclosporin A, Lumulse PGO®, and Clove oil.

Another embodiment comprises cyclosporin A, Mackester EGDS®, and Clove oil.

Another embodiment comprises cyclosporin A, Mackester EGMS®, and Clove oil.

Another embodiment comprises cyclosporin A, Mackester GSTP®, and Clove oil.

Another embodiment comprises cyclosporin A, Mackester Series®, and Clove oil.

Another embodiment comprises cyclosporin A, Magrabar PDG-50®, and Clove oil.

Another embodiment comprises cyclosporin A, Mapeg 6000 DS®, and Clove oil.

Another embodiment comprises cyclosporin A, Marlowet 4702®, and Clove oil.

Another embodiment comprises cyclosporin A, Monalube 305®, and Clove oil.

Another embodiment comprises cyclosporin A, Monalube 310®, and Clove oil.

Another embodiment comprises cyclosporin A, Monalube 315®, and Clove oil.

Another embodiment comprises cyclosporin A, Monalube 320®, and Clove oil.

Another embodiment comprises cyclosporin A, Monalube 325®, and Clove oil.

Another embodiment comprises cyclosporin A, Monalube 330®, and Clove oil.

Another embodiment comprises cyclosporin A, Naturechem PGHS®, and Clove oil.

Another embodiment comprises cyclosporin A, Polycastorol PLO-840®, and Clove oil.

Another embodiment comprises cyclosporin A, Polytex 10M®, and Clove oil.

Another embodiment comprises cyclosporin A, Ritasynt IP®, and Clove oil.

Another embodiment comprises cyclosporin A, Ross Chem PEG 600 DT®, and Clove oil.

Another embodiment comprises cyclosporin A, Schercemol PGMS®, and Clove oil.

Another embodiment comprises cyclosporin A, Sponto H-44C®, and Clove oil.

Another embodiment comprises cyclosporin A, Tegin G®, and Clove oil.

Another embodiment comprises cyclosporin A, Witbreak DGE-182®, and Clove oil.

Another embodiment comprises cyclosporin A, Witbreak DGE-182®, and Clove oil.

Another embodiment comprises cyclosporin A, Witbreak DRA-21®, and Clove oil.

Another embodiment comprises cyclosporin A, Witbreak DRA-21®, and Clove oil.

Another embodiment comprises cyclosporin A, Witbreak DRA-50®, and Clove oil.

Another embodiment comprises cyclosporin A, Witbreak DRA-50®, and Clove oil.

Another embodiment comprises cyclosporin A, Witconol F26-46®, and Clove oil.

Another embodiment comprises cyclosporin A, Witconol H-32®, and Clove oil.

Another embodiment comprises cyclosporin A, Witconol H-33®, and Clove oil.

Another embodiment comprises cyclosporin A, Witconol H-35A®, and Clove oil.

Another embodiment comprises cyclosporin A, Witconol RHP®, and Clove oil.

Another embodiment comprises cyclosporin A, a Lanolin-based Derivative, and Clove oil.

Another embodiment comprises cyclosporin A, Amerchol CAB®, and Clove oil.

Another embodiment comprises cyclosporin A, Amerchol L-101®, and Clove oil.

Another embodiment comprises cyclosporin A, Amerlate LFA-LO®, and Clove oil.

Another embodiment comprises cyclosporin A, Amerlate P®, and Clove oil.

Another embodiment comprises cyclosporin A, Barre Common Degras®, and Clove oil.

Another embodiment comprises cyclosporin A, Cholesterol NF®, and Clove oil.

Another embodiment comprises cyclosporin A, Crodalan AWS®, and Clove oil.

Another embodiment comprises cyclosporin A, Crodalan LA®, and Clove oil.

Another embodiment comprises cyclosporin A, Emery 1650®, and Clove oil.

Another embodiment comprises cyclosporin A, Emery 1650®, and Clove oil.

Another embodiment comprises cyclosporin A, Emery 1740®, and Clove oil.

Another embodiment comprises cyclosporin A, Emery 1740®, and Clove oil.

Another embodiment comprises cyclosporin A, Forlan 500®, and Clove oil.

Another embodiment comprises cyclosporin A, Forlan L®, and Clove oil.

Another embodiment comprises cyclosporin A, Laneto 50®, and Clove oil.

Another embodiment comprises cyclosporin A, Laneto 100®, and Clove oil.

Another embodiment comprises cyclosporin A, Laneto AWS®, and Clove oil.

Another embodiment comprises cyclosporin A, Lanfrax 1776®, and Clove oil.

Another embodiment comprises cyclosporin A, Lanfrax 1776®, and Clove oil.

Another embodiment comprises cyclosporin A, Lanogel 21®, and Clove oil.

Another embodiment comprises cyclosporin A, Lipolan®, and Clove oil.

Another embodiment comprises cyclosporin A, Lipolan 31®, and Clove oil.

Another embodiment comprises cyclosporin A, OHlan®, and Clove oil.

Another embodiment comprises cyclosporin A, Polychol 5®, and Clove oil.

Another embodiment comprises cyclosporin A, Polychol 15®, and Clove oil.

Another embodiment comprises cyclosporin A, Ritacetyl®, and Clove oil.

Another embodiment comprises cyclosporin A, Ritachol®, and Clove oil.

Another embodiment comprises cyclosporin A, Ritahydrox®, and Clove oil.

Another embodiment comprises cyclosporin A, Ritalafa®, and Clove oil.

Another embodiment comprises cyclosporin A, Ritalan®, and Clove oil.

Another embodiment comprises cyclosporin A, Ritalan AWS®, and Clove oil.

Another embodiment comprises cyclosporin A, Ritalan C®, and Clove oil.

Another embodiment comprises cyclosporin A, Ritawax®, and Clove oil.

Another embodiment comprises cyclosporin A, Ritawax AEO®, and Clove oil.

Another embodiment comprises cyclosporin A, Ritawax ALA®, and Clove oil.

Another embodiment comprises cyclosporin A, Solan/Solan 50/Super Solan®, and Clove oil.

Another embodiment comprises cyclosporin A, Super Hartolan/Hartolan®, and Clove oil.

Another embodiment comprises cyclosporin A, Supersat AWS-4®, and Clove oil.

Another embodiment comprises cyclosporin A, Supersat AWS-24®, and Clove oil.

Another embodiment comprises cyclosporin A, a Lecithin or Lecithin Derivative, and Clove oil.

Another embodiment comprises cyclosporin A, Alcolec®, and Clove oil.

Another embodiment comprises cyclosporin A, Lecithin®, and Clove oil.

Another embodiment comprises cyclosporin A, Lexin K®, and Clove oil.

Another embodiment comprises cyclosporin A, Natipide®, and Clove oil.

Another embodiment comprises cyclosporin A, a Lignin or Lignin Derivative, and Clove oil.

Another embodiment comprises cyclosporin A, Diwatex XP 9®, and Clove oil.

Another embodiment comprises cyclosporin A, Dynasperse LCD®, and Clove oil.

Another embodiment comprises cyclosporin A, Indulin SAL®, and Clove oil.

Another embodiment comprises cyclosporin A, Indulin W-1®, and Clove oil.

Another embodiment comprises cyclosporin A, Indulin W-5®, and Clove oil.

Another embodiment comprises cyclosporin A, Lignosol FTA®, and Clove oil.

Another embodiment comprises cyclosporin A, Lignosol SFX-65®, and Clove oil.

Another embodiment comprises cyclosporin A, Marasperse 52 CP®, and Clove oil.

Another embodiment comprises cyclosporin A, Marasperse AG®, and Clove oil.

Another embodiment comprises cyclosporin A, Marasperse CBOS-4®, and Clove oil.

Another embodiment comprises cyclosporin A, Ufoxane 2®, and Clove oil.

Another embodiment comprises cyclosporin A, a Methyl Ester, and Clove oil.

Another embodiment comprises cyclosporin A, E.B. Cleaner AK®, and Clove oil.

Another embodiment comprises cyclosporin A, Oleocal ME-70®, and Clove oil.

Another embodiment comprises cyclosporin A, Trade Name Company®, and Clove oil.

Another embodiment comprises cyclosporin A, Methyl Esters (cont'd)®, and Clove oil.

Another embodiment comprises cyclosporin A, Oleocal ME-92®, and Clove oil.

Another embodiment comprises cyclosporin A, Oleocal ME-112®, and Clove oil.

Another embodiment comprises cyclosporin A, Oleocal ME-130®, and Clove oil.

Another embodiment comprises cyclosporin A, a Monoglyceride or a Derivative thereof, and Clove oil.

Another embodiment comprises cyclosporin A, Dynacet 211®, and Clove oil.

Another embodiment comprises cyclosporin A, Hetsorb S-20®, and Clove oil.

Another embodiment comprises cyclosporin A, Imwitor 191®, and Clove oil.

Another embodiment comprises cyclosporin A, Imwitor 370®, and Clove oil.

Another embodiment comprises cyclosporin A, Imwitor 375®, and Clove oil.

Another embodiment comprises cyclosporin A, Imwitor 900®, and Clove oil.

Another embodiment comprises cyclosporin A, Imwitor 945®, and Clove oil.

Another embodiment comprises cyclosporin A, Imwitor 2020®, and Clove oil.

Another embodiment comprises cyclosporin A, Kemester 5500®, and Clove oil.

Another embodiment comprises cyclosporin A, Kemester 6000®, and Clove oil.

Another embodiment comprises cyclosporin A, Magrabar GMC®, and Clove oil.

Another embodiment comprises cyclosporin A, Magrabar GMO-CK®, and Clove oil.

Another embodiment comprises cyclosporin A, Magrabar GPC-10®, and Clove oil.

Another embodiment comprises cyclosporin A, Magrabar MDG-5050®, and Clove oil.

Another embodiment comprises cyclosporin A, Monalube 335®, and Clove oil.

Another embodiment comprises cyclosporin A, Rita GMS®, and Clove oil.

Another embodiment comprises cyclosporin A, Ritamulse SCG®, and Clove oil.

Another embodiment comprises cyclosporin A, Softigen 701®, and Clove oil.

Another embodiment comprises cyclosporin A, Tally 100 Plus®, and Clove oil.

Another embodiment comprises cyclosporin A, a Polyethylene Glycol, and Clove oil.

Another embodiment comprises cyclosporin A, Emulgade PL 68/50®, and Clove oil.

Another embodiment comprises cyclosporin A, Lumulse PEG®, and Clove oil.

Another embodiment comprises cyclosporin A, Rhodasurf PEG-400®, and Clove oil.

Another embodiment comprises cyclosporin A, Rhodasurf PEG-600®, and Clove oil.

Another embodiment comprises cyclosporin A, Witconol PEG-400®, and Clove oil.

Another embodiment comprises cyclosporin A, a Polymeric Surtactant, and Clove oil.

Another embodiment comprises cyclosporin A, Acritamer PNC-EG®, and Clove oil.

Another embodiment comprises cyclosporin A, Ag-Rho DEP-775®, and Clove oil.

Another embodiment comprises cyclosporin A, APG 325N Glycoside®, and Clove oil.

Another embodiment comprises cyclosporin A, Aristoflex AVC®, and Clove oil.

Another embodiment comprises cyclosporin A, Aristoflex HMB®, and Clove oil.

Another embodiment comprises cyclosporin A, Burco NPS-225®, and Clove oil.

Another embodiment comprises cyclosporin A, Burco NPS-816®, and Clove oil.

Another embodiment comprises cyclosporin A, Chemccinate 5603®, and Clove oil.

Another embodiment comprises cyclosporin A, Cosmedia Guar C-261N®, and Clove oil.

Another embodiment comprises cyclosporin A, Gantrez S-95®, and Clove oil.

Another embodiment comprises cyclosporin A, Glucopon 220 UP®, and Clove oil.

Another embodiment comprises cyclosporin A, Glucopon 225 DK®, and Clove oil.

Another embodiment comprises cyclosporin A, Glucopon 425 N®, and Clove oil.

Another embodiment comprises cyclosporin A, Glucopon 600 UP®, and Clove oil.

Another embodiment comprises cyclosporin A, Glucopon 625 UP®, and Clove oil.

Another embodiment comprises cyclosporin A, Pemulen 1621®, and Clove oil.

Another embodiment comprises cyclosporin A, Pemulen 1622®, and Clove oil.

Another embodiment comprises cyclosporin A, Pemulen TR-1®, and Clove oil.

Another embodiment comprises cyclosporin A, Pemulen TR-2®, and Clove oil.

Another embodiment comprises cyclosporin A, Plantacare 818®, and Clove oil.

Another embodiment comprises cyclosporin A, Plantapon LGC Sorb®, and Clove oil.

Another embodiment comprises cyclosporin A, Plantaren 1200N®, and Clove oil.

Another embodiment comprises cyclosporin A, Plantaren 2000N®, and Clove oil.

Another embodiment comprises cyclosporin A, Viscolam AT 64®, and Clove oil.

Another embodiment comprises cyclosporin A, Viscolam AT 64P®, and Clove oil.

Another embodiment comprises cyclosporin A, Viscolam AT 100®, and Clove oil.

Another embodiment comprises cyclosporin A, Viscolam MAC 7®, and Clove oil.

Another embodiment comprises cyclosporin A, Viscolam SMC 20®, and Clove oil.

Another embodiment comprises cyclosporin A, Witbreak RTC-323®, and Clove oil.

Another embodiment comprises cyclosporin A, WSI 3700®, and Clove oil.

Another embodiment comprises cyclosporin A, Antarox AA-60®, and Clove oil.

Another embodiment comprises cyclosporin A, Antarox LF-224®, and Clove oil.

Another embodiment comprises cyclosporin A, Burcomul DFE-45®, and Clove oil.

Another embodiment comprises cyclosporin A, Burcoterge LFE-1000®, and Clove oil.

Another embodiment comprises cyclosporin A, Chemal LF-25B®, and Clove oil.

Another embodiment comprises cyclosporin A, Chemal LF-40B®, and Clove oil.

Another embodiment comprises cyclosporin A, Dehypon LS-36®, and Clove oil.

Another embodiment comprises cyclosporin A, Dehypon LS-36G®, and Clove oil.

Another embodiment comprises cyclosporin A, Dehypon LS-54®, and Clove oil.

Another embodiment comprises cyclosporin A, Dehypon LS-54®, and Clove oil.

Another embodiment comprises cyclosporin A, Delonic 100 VLF®, and Clove oil.

Another embodiment comprises cyclosporin A, Delonic LF-60 MOD®, and Clove oil.

Another embodiment comprises cyclosporin A, Empiderm B®, and Clove oil.

Another embodiment comprises cyclosporin A, Ethylan 1206®, and Clove oil.

Another embodiment comprises cyclosporin A, Ethylan NS-500K®, and Clove oil.

Another embodiment comprises cyclosporin A, Ethylan NS-500LQ®, and Clove oil.

Another embodiment comprises cyclosporin A, Genapol 1392®, and Clove oil.

Another embodiment comprises cyclosporin A, Genapol 2317®, and Clove oil.

Another embodiment comprises cyclosporin A, Genapol 26EP710®, and Clove oil.

Another embodiment comprises cyclosporin A, Genapol EP 1022®, and Clove oil.

Another embodiment comprises cyclosporin A, Genapol EP 1024®, and Clove oil.

Another embodiment comprises cyclosporin A, Genapol EP 6068®, and Clove oil.

Another embodiment comprises cyclosporin A, Genapol NP915®, and Clove oil.

Another embodiment comprises cyclosporin A, Kieralon MFB®, and Clove oil.

Another embodiment comprises cyclosporin A, Lumisolve CSA-80 V®, and Clove oil.

Another embodiment comprises cyclosporin A, Marlowet 5001®, and Clove oil.

Another embodiment comprises cyclosporin A, Marlox FK 64®, and Clove oil.

Another embodiment comprises cyclosporin A, Marlox MO 124®, and Clove oil.

Another embodiment comprises cyclosporin A, Marlox S 58®, and Clove oil.

Another embodiment comprises cyclosporin A, Nonatell 1003®, and Clove oil.

Another embodiment comprises cyclosporin A, Nonatell 1038®, and Clove oil.

Another embodiment comprises cyclosporin A, Nonatell 1052®, and Clove oil.

Another embodiment comprises cyclosporin A, Nonatell 1061®, and Clove oil.

Another embodiment comprises cyclosporin A, Nonatell 1075®, and Clove oil.

Another embodiment comprises cyclosporin A, Nonatell 1088®, and Clove oil.

Another embodiment comprises cyclosporin A, Nonatell 1123®, and Clove oil.

Another embodiment comprises cyclosporin A, Nonatell 1153®, and Clove oil.

Another embodiment comprises cyclosporin A, Nonatell 1161®, and Clove oil.

Another embodiment comprises cyclosporin A, Nonatell 1172, and Clove oil.

Another embodiment comprises cyclosporin A, Nonatell 1181®, and Clove oil.

Another embodiment comprises cyclosporin A, Norfox 36®, and Clove oil.

Another embodiment comprises cyclosporin A, Procetyl AWS®, and Clove oil.

Another embodiment comprises cyclosporin A, Sandoxylate SX 412®, and Clove oil.

Another embodiment comprises cyclosporin A, Sandoxylate SX 418®, and Clove oil.

Another embodiment comprises cyclosporin A, Surfonic JL-80X®, and Clove oil.

Another embodiment comprises cyclosporin A, Surfonic JL-80X-B1®, and Clove oil.

Another embodiment comprises cyclosporin A, Surfonic L4-29X®, and Clove oil.

Another embodiment comprises cyclosporin A, Surfonic LF®, and Clove oil.

Another embodiment comprises cyclosporin A, T-Det A826®, and Clove oil.

Another embodiment comprises cyclosporin A, T-Det LF-416®, and Clove oil.

Another embodiment comprises cyclosporin A, Tergitol Min-Foam 1X®, and Clove oil.

Another embodiment comprises cyclosporin A, Tergitol Min-Foam 2X®, and Clove oil.

Another embodiment comprises cyclosporin A, Triton CF-21®, and Clove oil.

Another embodiment comprises cyclosporin A, Triton CF-76®, and Clove oil.

Another embodiment comprises cyclosporin A, Triton XL-80N®, and Clove oil.

Another embodiment comprises cyclosporin A, Witconol NS-98®, and Clove oil.

Another embodiment comprises cyclosporin A, Witconol NS-108LQ®, and Clove oil.

Another embodiment comprises cyclosporin A, Witconol NS-145®, and Clove oil.

Another embodiment comprises cyclosporin A, Witconol NS-179®, and Clove oil.

Another embodiment comprises cyclosporin A, a Protein-based Surfactant, and Clove oil.

Another embodiment comprises cyclosporin A, AminoFoam W®, and Clove oil.

Another embodiment comprises cyclosporin A, Amiter LGOD-2®, and Clove oil.

Another embodiment comprises cyclosporin A, Amiter LGS-2®, and Clove oil.

Another embodiment comprises cyclosporin A, Amiter LGS-5®, and Clove oil.

Another embodiment comprises cyclosporin A, Lamepon S®, and Clove oil.

Another embodiment comprises cyclosporin A, Lamepon S®, and Clove oil.

Another embodiment comprises cyclosporin A, Maypon 4C®, and Clove oil.

Another embodiment comprises cyclosporin A, May-Tein C®, and Clove oil.

Another embodiment comprises cyclosporin A, May-Tein CT®, and Clove oil.

Another embodiment comprises cyclosporin A, May-Tein KTS®, and Clove oil.

Another embodiment comprises cyclosporin A, May-Tein SY®, and Clove oil.

Another embodiment comprises cyclosporin A, Plantapon S®, and Clove oil.

Another embodiment comprises cyclosporin A, Proteol APL®, and Clove oil.

Another embodiment comprises cyclosporin A, Proteol OAT®, and Clove oil.

Another embodiment comprises cyclosporin A, Pyroter CPI-40®, and Clove oil.

Another embodiment comprises cyclosporin A, Pyroter GPI-25®, and Clove oil.

Another embodiment comprises cyclosporin A, Supro-Tein S®, and Clove oil.

Another embodiment comprises cyclosporin A, Supro-Tein V®, and Clove oil.

Another embodiment comprises cyclosporin A, a Sarcosine Derivative®, and Clove oil.

Another embodiment comprises cyclosporin A, Crodasinic LS-30®, and Clove oil.

Another embodiment comprises cyclosporin A, Vanseal CS®, and Clove oil.

Another embodiment comprises cyclosporin A, Vanseal LS®, and Clove oil.

Another embodiment comprises cyclosporin A, Vanseal MS®, and Clove oil.

Another embodiment comprises cyclosporin A, Vanseal NACS-30®, and Clove oil.

Another embodiment comprises cyclosporin A, Vanseal NALS-95®, and Clove oil.

Another embodiment comprises cyclosporin A, Vanseal OS®, and Clove oil.

Another embodiment comprises cyclosporin A, a Silicone-based Surfactant®, and Clove oil.

Another embodiment comprises cyclosporin A, Abil-B-9950®, and Clove oil.

Another embodiment comprises cyclosporin A, Abil Care 85®, and Clove oil.

Another embodiment comprises cyclosporin A, Abil EM 90®, and Clove oil.

Another embodiment comprises cyclosporin A, Abil EM 97®, and Clove oil.

Another embodiment comprises cyclosporin A, Abil WE-09®, and Clove oil.

Another embodiment comprises cyclosporin A, Dow Corning 1248 Fluid®, and Clove oil.

Another embodiment comprises cyclosporin A, Dow Corning 3225C®, and Clove oil.

Another embodiment comprises cyclosporin A, Dow Corning 5200®, and Clove oil.

Another embodiment comprises cyclosporin A, Dow Corning Q4-3667®, and Clove oil.

Another embodiment comprises cyclosporin A, Monasil PCA®, and Clove oil.

Another embodiment comprises cyclosporin A, Monasil PDM®, and Clove oil.

Another embodiment comprises cyclosporin A, Monasil PLN®, and Clove oil.

Another embodiment comprises cyclosporin A, Polyderm PPI-SI-WS®, and Clove oil.

Another embodiment comprises cyclosporin A, Troysol 380W®, and Clove oil.

Another embodiment comprises cyclosporin A, Troysol S366®, and Clove oil.

Another embodiment comprises cyclosporin A, a Sorbitan Derivative, and Clove oil.

Another embodiment comprises cyclosporin A, Alkamuls SML®, and Clove oil.

Another embodiment comprises cyclosporin A, Alkamuls SMO®, and Clove oil.

Another embodiment comprises cyclosporin A, Alkamuls STO®, and Clove oil.

Another embodiment comprises cyclosporin A, Arlacel 20®, and Clove oil.

Another embodiment comprises cyclosporin A, Arlacel 40®, and Clove oil.

Another embodiment comprises cyclosporin A, Arlacel 60®, and Clove oil.

Another embodiment comprises cyclosporin A, Arlacel 80®, and Clove oil.

Another embodiment comprises cyclosporin A, Arlacel C®, and Clove oil.

Another embodiment comprises cyclosporin A, Armul 21®, and Clove oil.

Another embodiment comprises cyclosporin A, Atlox 80®, and Clove oil.

Another embodiment comprises cyclosporin A, Atlox 847®, and Clove oil.

Another embodiment comprises cyclosporin A, Atlox 1045A®, and Clove oil.

Another embodiment comprises cyclosporin A, Canarcel 20®, and Clove oil.

Another embodiment comprises cyclosporin A, Canarcel 60®, and Clove oil.

Another embodiment comprises cyclosporin A, Canarcel 80®, and Clove oil.

Another embodiment comprises cyclosporin A, Canarcel TW 20®, and Clove oil.

Another embodiment comprises cyclosporin A, Canarcel TW 60®, and Clove oil.

Another embodiment comprises cyclosporin A, Canarcel TW 80®, and Clove oil.

Another embodiment comprises cyclosporin A, Coladet BSB-P®, and Clove oil.

Another embodiment comprises cyclosporin A, Customulse O-20®, and Clove oil.

Another embodiment comprises cyclosporin A, Dehymuls E®s and Clove oil.

Another embodiment comprises cyclosporin A, DeSotan SMO®, and Clove oil.

Another embodiment comprises cyclosporin A, DeSotan SMO-20®, and Clove oil.

Another embodiment comprises cyclosporin A, DeSotan SMT®, and Clove oil.

Another embodiment comprises cyclosporin A, DeSotan SMT-20®, and Clove oil.

Another embodiment comprises cyclosporin A, Durfax 60®, and Clove oil.

Another embodiment comprises cyclosporin A, Durfax 65®, and Clove oil.

Another embodiment comprises cyclosporin A, Durfax 80®, and Clove oil.

Another embodiment comprises cyclosporin A, Durtan 60®, and Clove oil.

Another embodiment comprises cyclosporin A, Durtan 65®, and Clove oil.

Another embodiment comprises cyclosporin A, Liposorb L®, and Clove oil.

Another embodiment comprises cyclosporin A, Liposorb L-10®, and Clove oil.

Another embodiment comprises cyclosporin A, Liposorb L-20®, and Clove oil.

Another embodiment comprises cyclosporin A, Liposorb O®, and Clove oil.

Another embodiment comprises cyclosporin A, Liposorb O-20®, and Clove oil.

Another embodiment comprises cyclosporin A, Liposorb P®, and Clove oil.

Another embodiment comprises cyclosporin A, Liposorb P-20®, and Clove oil.

Another embodiment comprises cyclosporin A, Liposorb S®, and Clove oil.

Another embodiment comprises cyclosporin A, Liposorb S-20®, and Clove oil.

Another embodiment comprises cyclosporin A, Liposorb SQO®, and Clove oil.

Another embodiment comprises cyclosporin A, Liposorb TO®, and Clove oil.

Another embodiment comprises cyclosporin A, Liposorb TS®, and Clove oil.

Another embodiment comprises cyclosporin A, Liposorb TS-20®, and Clove oil.

Another embodiment comprises cyclosporin A, Lumisorb PS®, and Clove oil.

Another embodiment comprises cyclosporin A, Lumisorb SMO (T)®, and Clove oil.

Another embodiment comprises cyclosporin A, Lumisorb SMS K®, and Clove oil.

Another embodiment comprises cyclosporin A, Lumisorb SSO®, and Clove oil.

Another embodiment comprises cyclosporin A, Lumisorb STS K®, and Clove oil.

Another embodiment comprises cyclosporin A, Lumisorb STT®, and Clove oil.

Another embodiment comprises cyclosporin A, Magrabar SMO®, and Clove oil.

Another embodiment comprises cyclosporin A, Magrabar SMO-VEG®, and Clove oil.

Another embodiment comprises cyclosporin A, Magrabar SMT®, and Clove oil.

Another embodiment comprises cyclosporin A, Magrabar STO®, and Clove oil.

Another embodiment comprises cyclosporin A, Miracare BC-27®, and Clove oil.

Another embodiment comprises cyclosporin A, Ritabate 20®, and Clove oil.

Another embodiment comprises cyclosporin A, Ritabate 40®, and Clove oil.

Another embodiment comprises cyclosporin A, Ritabate 60®, and Clove oil.

Another embodiment comprises cyclosporin A, Ritabate 80®, and Clove oil.

Another embodiment comprises cyclosporin A, T-Maz®, and Clove oil.

Another embodiment comprises cyclosporin A, Tego SML®, and Clove oil.

Another embodiment comprises cyclosporin A, Tego SML 20®, and Clove oil.

Another embodiment comprises cyclosporin A, Tego SMO 80 V®, and Clove oil.

Another embodiment comprises cyclosporin A, Tego SMO V®, and Clove oil.

Another embodiment comprises cyclosporin A, Tego SMS®, and Clove oil.

Another embodiment comprises cyclosporin A, Tego STO V®, and Clove oil.

Another embodiment comprises cyclosporin A, Tween 21®, and Clove oil.

Another embodiment comprises cyclosporin A, Tween 40®, and Clove oil.

Another embodiment comprises cyclosporin A, Tween 60®, and Clove oil.

Another embodiment comprises cyclosporin A, Tween 60 K®, and Clove oil.

Another embodiment comprises cyclosporin A, Tween 61®, and Clove oil.

Another embodiment comprises cyclosporin A, Tween 65®, and Clove oil.

Another embodiment comprises cyclosporin A, Tween 80®, and Clove oil.

Another embodiment comprises cyclosporin A, Tween 80 K®, and Clove oil.

Another embodiment comprises cyclosporin A, Tween 81®, and Clove oil.

Another embodiment comprises cyclosporin A, Tween 85®, and Clove oil.

Another embodiment comprises cyclosporin A, a Sucrose or Glucose Ester, or Derivative thereof, and Clove oil.

Another embodiment comprises cyclosporin A, DeSulf GOS-P-60WCG®, and Clove oil.

Another embodiment comprises cyclosporin A, Glucam E-20 Distearate®, and Clove oil.

Another embodiment comprises cyclosporin A, Glucamate DOE-120®, and Clove oil.

Another embodiment comprises cyclosporin A, Glucamate SSE-20®, and Clove oil.

Another embodiment comprises cyclosporin A, Glucate DO®, and Clove oil.

Another embodiment comprises cyclosporin A, Glucate SS®, and Clove oil.

Another embodiment comprises cyclosporin A, Glucopon 425 UP®, and Clove oil.

Another embodiment comprises cyclosporin A, Isolan IS®, and Clove oil.

Another embodiment comprises cyclosporin A, Mazon 40®, and Clove oil.

Another embodiment comprises cyclosporin A, Montanov 82®, and Clove oil.

Another embodiment comprises cyclosporin A, Montanov 202®, and Clove oil.

Another embodiment comprises cyclosporin A, Montanov S®, and Clove oil.

Another embodiment comprises cyclosporin A, Rheozan®, and Clove oil.

Another embodiment comprises cyclosporin A, Simulsol AS 48®, and Clove oil.

Another embodiment comprises cyclosporin A, Simulsol SL 4®, and Clove oil.

Another embodiment comprises cyclosporin A, Simulsol SL 10®, and Clove oil.

Another embodiment comprises cyclosporin A, Simulsol SL 11W®, and Clove oil.

Another embodiment comprises cyclosporin A, Simulsol SL 55®, and Clove oil.

Another embodiment comprises cyclosporin A, Suga Nate 100 and 160®, and Clove oil.

Another embodiment comprises cyclosporin A, Tego Care 450®, and Clove oil.

Another embodiment comprises cyclosporin A, Tego Care CG 90®, and Clove oil.

Another embodiment comprises cyclosporin A, Tego Care PS®, and Clove oil.

Another embodiment comprises cyclosporin A, Tegosoft PSE 141 G®, and Clove oil.

Another embodiment comprises cyclosporin A, Tegotens G 826®, and Clove oil.

Another embodiment comprises cyclosporin A, Triton BG-10 (70%)®, and Clove oil.

Another embodiment comprises cyclosporin A, Triton CG-110 (60%)®, and Clove oil.

Another embodiment comprises cyclosporin A, Wickenol 545®, and Clove oil.

Another embodiment comprises cyclosporin A, Diglycerol®, and Cassia oil.

Another embodiment comprises cyclosporin A, Hetoxide GT-80®, and Cassia oil.

Another embodiment comprises cyclosporin A, Lexemul BEO®, and Cassia oil.

Another embodiment comprises cyclosporin A, Polyglycerol-3®, and Cassia oil.

Another embodiment comprises cyclosporin A, Redicote E Series®, and Cassia oil.

Another embodiment comprises cyclosporin A, Simulsol OX 1005L®, and Cassia oil.

Another embodiment comprises cyclosporin A, Stanfax 567®, and Cassia oil.

Another embodiment comprises cyclosporin A, TA-1618®, and Cassia oil.

Another embodiment comprises cyclosporin A, Witconol H-31A®, and Cassia oil.

Another embodiment comprises cyclosporin A, AO-405®, and Cassia oil.

Another embodiment comprises cyclosporin A, AO-455®, and Cassia oil.

Another embodiment comprises cyclosporin A, AO 728 Special®, and Cassia oil.

Another embodiment comprises cyclosporin A, Barlox 12®, and Cassia oil.

Another embodiment comprises cyclosporin A, Barlox 14®, and Cassia oil.

Another embodiment comprises cyclosporin A, Burcoxide Lo®, and Cassia oil.

Another embodiment comprises cyclosporin A, Caloxamine LO®, and Cassia oil.

Another embodiment comprises cyclosporin A, Chemoxide CAW®, and Cassia oil.

Another embodiment comprises cyclosporin A, Chemoxide LM-30n, and Cassia oil.

Another embodiment comprises cyclosporin A, Chemoxide LO®, and Cassia oil.

Another embodiment comprises cyclosporin A, Chemoxide MO®, and Cassia oil.

Another embodiment comprises cyclosporin A, Colalux CAO-35®, and Cassia oil.

Another embodiment comprises cyclosporin A, Colalux LO®, and Cassia oil.

Another embodiment comprises cyclosporin A, DeMox CAPO®, and Cassia oil.

Another embodiment comprises cyclosporin A, DeMox CSG-30®, and Cassia oil.

Another embodiment comprises cyclosporin A, DeMox LAO®, and Cassia oil.

Another embodiment comprises cyclosporin A, Emcol L®, and Cassia oil.

Another embodiment comprises cyclosporin A, Empigen OB®, and Cassia oil.

Another embodiment comprises cyclosporin A, Empigen OS/A®, and Cassia oil.

Another embodiment comprises cyclosporin A, Foamox CDO®, and Cassia oil.

Another embodiment comprises cyclosporin A, Foamox DMM®, and Cassia oil.

Another embodiment comprises cyclosporin A, Foamox DMS®, and Cassia oil.

Another embodiment comprises cyclosporin A, Genaminox KC®, and Cassia oil.

Another embodiment comprises cyclosporin A, Genaminox LA®, and Cassia oil.

Another embodiment comprises cyclosporin A, Hartofoam SAO®, and Cassia oil.

Another embodiment comprises cyclosporin A, Hartox DMCD®, and Cassia oil.

Another embodiment comprises cyclosporin A, Lipowax DAT®, and Cassia oil.

Another embodiment comprises cyclosporin A, Lipowax PB Pastilles®, and Cassia oil.

Another embodiment comprises cyclosporin A, Mackamine C8®, and Cassia oil.

Another embodiment comprises cyclosporin A, Mackamine C10®, and Cassia oil.

Another embodiment comprises cyclosporin A, Mackamine C14®, and Cassia oil.

Another embodiment comprises cyclosporin A, Mackamine CAO®, and Cassia oil.

Another embodiment comprises cyclosporin A, Mackamine CO®, and Cassia oil.

Another embodiment comprises cyclosporin A, Mackamine LO®, and Cassia oil.

Another embodiment comprises cyclosporin A, Mackamine O2®, and Cassia oil.

Another embodiment comprises cyclosporin A, Mackamine SAO®, and Cassia oil.

Another embodiment comprises cyclosporin A, Mackamine SO®, and Cassia oil.

Another embodiment comprises cyclosporin A, Mazox KCAO®, and Cassia oil.

Another embodiment comprises cyclosporin A, Monalac MO®, and Cassia oil.

Another embodiment comprises cyclosporin A, Norfox LDA®, and Cassia oil.

Another embodiment comprises cyclosporin A, Rhodamox LO®, and Cassia oil.

Another embodiment comprises cyclosporin A, Schercamox C-AA®, and Cassia oil.

Another embodiment comprises cyclosporin A, Schercamox DMA®, and Cassia oil.

Another embodiment comprises cyclosporin A, Schercamox DML®, and Cassia oil.

Another embodiment comprises cyclosporin A, Schercamox DMM®, and Cassia oil.

Another embodiment comprises cyclosporin A, Schercamox DMS®, and Cassia oil.

Another embodiment comprises cyclosporin A, Tegotens DO®, and Cassia oil.

Another embodiment comprises cyclosporin A, Tomah AO-14-2®, and Cassia oil.

Another embodiment comprises cyclosporin A, Triaminox CDO®, and Cassia oil.

Another embodiment comprises cyclosporin A, a block polymer, and Cassia oil.

Another embodiment comprises cyclosporin A, AL 2070®, and Cassia oil.

Another embodiment comprises cyclosporin A, Antarox 17-R-2®, and Cassia oil.

Another embodiment comprises cyclosporin A, Antarox 25-R-2®, and Cassia oil.

Another embodiment comprises cyclosporin A, Antarox 31-R-1®, and Cassia oil.

Another embodiment comprises cyclosporin A, Antarox P-84®, and Cassia oil.

Another embodiment comprises cyclosporin A, Antarox P-104/H®, and Cassia oil.

Another embodiment comprises cyclosporin A, Arnox BP-Series®, and Cassia oil.

Another embodiment comprises cyclosporin A, Chemonic 435®, and Cassia oil.

Another embodiment comprises cyclosporin A, Chemonic D-25®, and Cassia oil.

Another embodiment comprises cyclosporin A, Chemonic PL Series®, and Cassia oil.

Another embodiment comprises cyclosporin A, Ethox L-121®, and Cassia oil.

Another embodiment comprises cyclosporin A, Ethox L-122®, and Cassia oil.

Another embodiment comprises cyclosporin A, Genapol PF-10®, and Cassia oil.

Another embodiment comprises cyclosporin A, Genapol PF-20®, and Cassia oil.

Another embodiment comprises cyclosporin A, Genapol PF-40AA, and Cassia oil.

Another embodiment comprises cyclosporin A, Norfox 2-LF®, and Cassia oil.

Another embodiment comprises cyclosporin A, Simulsol NW 342®, and Cassia oil.

Another embodiment comprises cyclosporin A, T-Det BP-1®, and Cassia oil.

Another embodiment comprises cyclosporin A, T-Det XD®, and Cassia oil.

Another embodiment comprises cyclosporin A, T-Det XH®, and Cassia oil.

Another embodiment comprises cyclosporin A, Triton CF-32®, and Cassia oil.

Another embodiment comprises cyclosporin A, Witconol 171®, and Cassia oil.

Another embodiment comprises cyclosporin A, Witconol 324®, and Cassia oil.

Another embodiment comprises cyclosporin A, Witconol 324D®, and Cassia oil.

Another embodiment comprises cyclosporin A, Witconol PD-2000®, and Cassia oil.

Another embodiment comprises cyclosporin A, a Carboxylated Alcohol or Alkylphenol Ethoxylate, and Cassia oil.

Another embodiment comprises cyclosporin A, Emcol CN-6®, and Cassia oil.

Another embodiment comprises cyclosporin A, Ethcarb®, and Cassia oil.

Another embodiment comprises cyclosporin A, Gemtex WNT-Conc®, and Cassia oil.

Another embodiment comprises cyclosporin A, Incrodet TD7-C®, and Cassia oil.

Another embodiment comprises cyclosporin A, Marlinat CM 105/80®, and Cassia oil.

Another embodiment comprises cyclosporin A, Marlowet 1072®, and Cassia oil.

Another embodiment comprises cyclosporin A, Marlowet 4530®, and Cassia oil.

Another embodiment comprises cyclosporin A, Marlowet 4530 LF®, and Cassia oil.

Another embodiment comprises cyclosporin A, Marlowet 4534®, and Cassia oil.

Another embodiment comprises cyclosporin A, Marlowet 4538®, and Cassia oil.

Another embodiment comprises cyclosporin A, Marlowet 4539®, and Cassia oil.

Another embodiment comprises cyclosporin A, Marlowet 4539 LF®, and Cassia oil.

Another embodiment comprises cyclosporin A, Marlowet 4541®, and Cassia oil.

Another embodiment comprises cyclosporin A, Miranate LEC-80®, and Cassia oil.

Another embodiment comprises cyclosporin A, Sandopan B®, and Cassia oil.

Another embodiment comprises cyclosporin A, Sandopan B Modified®, and Cassia oil.

Another embodiment comprises cyclosporin A, Sandopan LS-24 Gel®, and Cassia oil.

Another embodiment comprises cyclosporin A, Sunine T-A®, and Cassia oil.

Another embodiment comprises cyclosporin A, a Carboxylic Acid/Fatty Acid, and Cassia oil.

Another embodiment comprises cyclosporin A, Colaterge RAM®, and Cassia oil.

Another embodiment comprises cyclosporin A, Colatrope INC®, and Cassia oil.

Another embodiment comprises cyclosporin A, Crodacid B®, and Cassia oil.

Another embodiment comprises cyclosporin A, DeTrope CA-100®, and Cassia oil.

Another embodiment comprises cyclosporin A, Latol MTO®, and Cassia oil.

Another embodiment comprises cyclosporin A, Lumulse CC-33 K®, and Cassia oil.

Another embodiment comprises cyclosporin A, Mulls 2218®, and Cassia oil.

Another embodiment comprises cyclosporin A, OL-600®, and Cassia oil.

Another embodiment comprises cyclosporin A, OL-800®, and Cassia oil.

Another embodiment comprises cyclosporin A, R-910®, and Cassia oil.

Another embodiment comprises cyclosporin A, S-210®, and Cassia oil.

Another embodiment comprises cyclosporin A, Sandopan DTC Acid®, and Cassia oil.

Another embodiment comprises cyclosporin A, Sandopan LS 24 N®, and Cassia oil.

Another embodiment comprises cyclosporin A, Sandopan MA-18®, and Cassia oil.

Another embodiment comprises cyclosporin A, an Ethoxylated Alcohol, and Cassia oil.

Another embodiment comprises cyclosporin A, Adsee 799®, and Cassia oil.

Another embodiment comprises cyclosporin A, Adsee 799®, and Cassia oil.

Another embodiment comprises cyclosporin A, Alfonic 610-3.5®, and Cassia oil.

Another embodiment comprises cyclosporin A, Alfonic 810-2®, and Cassia oil.

Another embodiment comprises cyclosporin A, Alfonic 810-6®, and Cassia oil.

Another embodiment comprises cyclosporin A, Alfonic 1012-3®, and Cassia oil.

Another embodiment comprises cyclosporin A, Alfonic 1012-5®, and Cassia oil.

Another embodiment comprises cyclosporin A, Alfonic 1216CO-1.5®, and Cassia oil.

Another embodiment comprises cyclosporin A, Alfonic 1216CO-7®, and Cassia oil.

Another embodiment comprises cyclosporin A, Alfonic 1412-3®, and Cassia oil.

Another embodiment comprises cyclosporin A, Alfonic 1412-7®, and Cassia oil.

Another embodiment comprises cyclosporin A, Arlasolve 200®, and Cassia oil.

Another embodiment comprises cyclosporin A, Arlasolve 200 Liquid®, and Cassia oil.

Another embodiment comprises cyclosporin A, Armix 180-C®, and Cassia oil.

Another embodiment comprises cyclosporin A, Armix 183®, and Cassia oil.

Another embodiment comprises cyclosporin A, Armul 2404®, and Cassia oil.

Another embodiment comprises cyclosporin A, Armul 2404®, and Cassia oil.

Another embodiment comprises cyclosporin A, Atlas EMJ-C®, and Cassia oil.

Another embodiment comprises cyclosporin A, Atlas G-2109®, and Cassia oil.

Another embodiment comprises cyclosporin A, Atlas G-3886®, and Cassia oil.

Another embodiment comprises cyclosporin A, Atlas G-3890®, and Cassia oil.

Another embodiment comprises cyclosporin A, Bio Soft E-200®, and Cassia oil.

Another embodiment comprises cyclosporin A, Bio Soft E-300®, and Cassia oil.

Another embodiment comprises cyclosporin A, Bio Soft E-400®, and Cassia oil.

Another embodiment comprises cyclosporin A, Bio Soft EN 600®, and Cassia oil.

Another embodiment comprises cyclosporin A, Bio Soft TD-400®, and Cassia oil.

Another embodiment comprises cyclosporin A, Bio Soft TD-630®, and Cassia oil.

Another embodiment comprises cyclosporin A, Brij 30®, and Cassia oil.

Another embodiment comprises cyclosporin A, Brij 52®, and Cassia oil.

Another embodiment comprises cyclosporin A, Brij 56®, and Cassia oil.

Another embodiment comprises cyclosporin A, Brij 58®, and Cassia oil.

Another embodiment comprises cyclosporin A, Brij 72®, and Cassia oil.

Another embodiment comprises cyclosporin A, Brij 76®, and Cassia oil.

Another embodiment comprises cyclosporin A, Brij 78®, and Cassia oil.

Another embodiment comprises cyclosporin A, Brij 93®, and Cassia oil.

Another embodiment comprises cyclosporin A, Brij 97®, and Cassia oil.

Another embodiment comprises cyclosporin A, Brij 98®, and Cassia oil.

Another embodiment comprises cyclosporin A, Brij 700®, and Cassia oil.

Another embodiment comprises cyclosporin A, Brij 700 S®, and Cassia oil.

Another embodiment comprises cyclosporin A, Brij 721®, and Cassia oil.

Another embodiment comprises cyclosporin A, Brij 721 S®, and Cassia oil.

Another embodiment comprises cyclosporin A, Burcoterge CDG®, and Cassia oil.

Another embodiment comprises cyclosporin A, Canasol AT 600®, and Cassia oil.

Another embodiment comprises cyclosporin A, Canasol AT 800®, and Cassia oil.

Another embodiment comprises cyclosporin A, Canasol AT 1200®, and Cassia oil.

Another embodiment comprises cyclosporin A, Canasol BJ 35®, and Cassia oil.

Another embodiment comprises cyclosporin A, Canasol BJ 36®, and Cassia oil.

Another embodiment comprises cyclosporin A, Canasol BJ 52®, and Cassia oil.

Another embodiment comprises cyclosporin A, Canasol BJ 58®, and Cassia oil.

Another embodiment comprises cyclosporin A, Canasol BJ 72®, and Cassia oil.

Another embodiment comprises cyclosporin A, Canasol BJ 78®, and Cassia oil.

Another embodiment comprises cyclosporin A, Canasol BJ 98®, and Cassia oil.

Another embodiment comprises cyclosporin A, Canasol BJ 307®, and Cassia oil.

Another embodiment comprises cyclosporin A, Certak 1400®, and Cassia oil.

Another embodiment comprises cyclosporin A, Cetomacrogol 1000 BP®, and Cassia oil.

Another embodiment comprises cyclosporin A, Chemonic C-2®, and Cassia oil.

Another embodiment comprises cyclosporin A, Chemonic C-10®, and Cassia oil.

Another embodiment comprises cyclosporin A, Chemonic C-20®, and Cassia oil.

Another embodiment comprises cyclosporin A, Chemonic CT-12®, and Cassia oil.

Another embodiment comprises cyclosporin A, Chembnic CT-20®, and Cassia oil.

Another embodiment comprises cyclosporin A, Chemonic CT-30®, and Cassia oil.

Another embodiment comprises cyclosporin A, Chemonic CT-55®, and Cassia oil.

Another embodiment comprises cyclosporin A, Chemonic G-7®, and Cassia oil.

Another embodiment comprises cyclosporin A, Chemonic G-26®, and Cassia oil.

Another embodiment comprises cyclosporin A, Chemonic L-4®, and Cassia oil.

Another embodiment comprises cyclosporin A, Chemonic L-7®, and Cassia oil.

Another embodiment comprises cyclosporin A, Chemonic L-12®, and Cassia oil.

Another embodiment comprises cyclosporin A, Chemonic L-23®, and Cassia oil.

Another embodiment comprises cyclosporin A, Chemonic O-2®, and Cassia oil.

Another embodiment comprises cyclosporin A, Chemonic O-5®, and Cassia oil.

Another embodiment comprises cyclosporin A, Chemonic O-10®, and Cassia oil.

Another embodiment comprises cyclosporin A, Chemonic O-20®, and Cassia oil.

Another embodiment comprises cyclosporin A, Chemonic S-2®, and Cassia oil.

Another embodiment comprises cyclosporin A, Chemonic S-10®, and Cassia oil.

Another embodiment comprises cyclosporin A, Chemonic S-20®, and Cassia oil.

Another embodiment comprises cyclosporin A, Colamulse FE®, and Cassia oil.

Another embodiment comprises cyclosporin A, Cremophor A 20®, and Cassia oil.

Another embodiment comprises cyclosporin A, Cremophor SA 2®, and Cassia oil.

Another embodiment comprises cyclosporin A, Dehydol 100®, and Cassia oil.

Another embodiment comprises cyclosporin A, Dehydol O-4®, and Cassia oil.

Another embodiment comprises cyclosporin A, Delonic C-18®, and Cassia oil.

Another embodiment comprises cyclosporin A, DeSonic 6T®, and Cassia oil.

Another embodiment comprises cyclosporin A, DeSonic 9D®, and Cassia oil.

Another embodiment comprises cyclosporin A, DeSonic 9T®, and Cassia oil.

Another embodiment comprises cyclosporin A, DeSonic 12D®, and Cassia oil.

Another embodiment comprises cyclosporin A, DeSonic 12T®, and Cassia oil.

Another embodiment comprises cyclosporin A, DeSonic 15T®, and Cassia oil.

Another embodiment comprises cyclosporin A, DeSonic TDA-9®, and Cassia oil.

Another embodiment comprises cyclosporin A, DeThox GLG-7®, and Cassia oil.

Another embodiment comprises cyclosporin A, DeThox GLG-26®, and Cassia oil.

Another embodiment comprises cyclosporin A, DeThox LA-4®, and Cassia oil.

Another embodiment comprises cyclosporin A, DeThox LA-23®, and Cassia oil.

Another embodiment comprises cyclosporin A, DeThox SA-80®, and Cassia oil.

Another embodiment comprises cyclosporin A, Disponil O5®, and Cassia oil.

Another embodiment comprises cyclosporin A, Eccoterge EO-41 B®, and Cassia oil.

Another embodiment comprises cyclosporin A, Empilan KA2.5/90®, and Cassia oil.

Another embodiment comprises cyclosporin A, Empilan KA5/90®, and Cassia oil.

Another embodiment comprises cyclosporin A, Empilan KM-20®, and Cassia oil.

Another embodiment comprises cyclosporin A, Empilan KM-50®, and Cassia oil.

Another embodiment comprises cyclosporin A, Empilan L-23®, and Cassia oil.

Another embodiment comprises cyclosporin A, Ethylan 25-3®, and Cassia oil.

Another embodiment comprises cyclosporin A, Ethylan 1204®, and Cassia oil.

Another embodiment comprises cyclosporin A, Ethylan DA-4®, and Cassia oil.

Another embodiment comprises cyclosporin A, Ethylan LA-230®, and Cassia oil.

Another embodiment comprises cyclosporin A, Ethylan SN®, and Cassia oil.

Another embodiment comprises cyclosporin A, Ethylan TD-60®, and Cassia oil.

Another embodiment comprises cyclosporin A, Ethylan TD-100®, and Cassia oil.

Another embodiment comprises cyclosporin A, Ethylan TD-1407®, and Cassia oil.

Another embodiment comprises cyclosporin A, Eumulgin B1®, and Cassia oil.

Another embodiment comprises cyclosporin A, Eumulgin B2®, and Cassia oil.

Another embodiment comprises cyclosporin A, Eumulgin B3®, and Cassia oil.

Another embodiment comprises cyclosporin A, Eumulgin O-10®, and Cassia oil.

Another embodiment comprises cyclosporin A, Flo Mo 80/20®, and Cassia oil.

Another embodiment comprises cyclosporin A, Flo Mo Low Foam®, and Cassia oil.

Another embodiment comprises cyclosporin A, Forlan C-24®, and Cassia oil.

Another embodiment comprises cyclosporin A, Genapol 1454®, and Cassia oil.

Another embodiment comprises cyclosporin A, Genapol BA-020®, and Cassia oil.

Another embodiment comprises cyclosporin A, Genapol BA-040®, and Cassia oil.

Another embodiment comprises cyclosporin A, Genapol C-100®, and Cassia oil.

Another embodiment comprises cyclosporin A, Genapol DA 060®, and Cassia oil.

Another embodiment comprises cyclosporin A, Genapol HS 020®, and Cassia oil.

Another embodiment comprises cyclosporin A, Genapol HS 200®, and Cassia oil.

Another embodiment comprises cyclosporin A, Genapol ID-040®, and Cassia oil.

Another embodiment comprises cyclosporin A, Genapol ID-060®, and Cassia oil.

Another embodiment comprises cyclosporin A, Genapol ID-090®, and Cassia oil.

Another embodiment comprises cyclosporin A, Genapol LA 010®, and Cassia oil.

Another embodiment comprises cyclosporin A, Genapol LA 020®, and Cassia oil.

Another embodiment comprises cyclosporin A, Genapol LA 030®, and Cassia oil.

Another embodiment comprises cyclosporin A, Genapol LA 040®, and Cassia oil.

Another embodiment comprises cyclosporin A, Genapol LA 050®, and Cassia oil.

Another embodiment comprises cyclosporin A, Genapol LA 060®, and Cassia oil.

Another embodiment comprises cyclosporin A, Genapol LA 070®, and Cassia oil.

Another embodiment comprises cyclosporin A, Genapol LA 070S®, and Cassia oil.

Another embodiment comprises cyclosporin A, Genapol LA 230®, and Cassia oil.

Another embodiment comprises cyclosporin A, Genapol O020®, and Cassia oil.

Another embodiment comprises cyclosporin A, Genapol O050®, and Cassia oil.

Another embodiment comprises cyclosporin A, Genapol O100®, and Cassia oil.

Another embodiment comprises cyclosporin A, Genapol O200®, and Cassia oil.

Another embodiment comprises cyclosporin A, Genapol SA 030®, and Cassia oil.

Another embodiment comprises cyclosporin A, Genapol SA 120®, and Cassia oil.

Another embodiment comprises cyclosporin A, Genapol T-020®, and Cassia oil.

Another embodiment comprises cyclosporin A, Genapol UD-030®, and Cassia oil.

Another embodiment comprises cyclosporin A, Genapol UD-050®, and Cassia oil.

Another embodiment comprises cyclosporin A, Genapol UD-070®, and Cassia oil.

Another embodiment comprises cyclosporin A, Genapol UD-079®, and Cassia oil.

Another embodiment comprises cyclosporin A, Genapol UD-080®, and Cassia oil.

Another embodiment comprises cyclosporin A, Genapol UD-110®, and Cassia oil.

Another embodiment comprises cyclosporin A, Genapol X 030®, and Cassia oil.

Another embodiment comprises cyclosporin A, Genapol X 050®, and Cassia oil.

Another embodiment comprises cyclosporin A, Genapol X 060®, and Cassia oil.

Another embodiment comprises cyclosporin A, Genapol X 070®, and Cassia oil.

Another embodiment comprises cyclosporin A, Genapol X 080®, and Cassia oil.

Another embodiment comprises cyclosporin A, Genapol X 100®, and Cassia oil.

Another embodiment comprises cyclosporin A, Genapol X159®, and Cassia oil.

Another embodiment comprises cyclosporin A, Generol 122 E5®, and Cassia oil.

Another embodiment comprises cyclosporin A, Generol 122 E25®, and Cassia oil.

Another embodiment comprises cyclosporin A, Hostacerin T-3®, and Cassia oil.

Another embodiment comprises cyclosporin A, Iconol LF 110®, and Cassia oil.

Another embodiment comprises cyclosporin A, Incropol CS-20®, and Cassia oil.

Another embodiment comprises cyclosporin A, Lexemul CS-20®, and Cassia oil.

Another embodiment comprises cyclosporin A, Liponic EG-1®, and Cassia oil.

Another embodiment comprises cyclosporin A, Lipowax D®, and Cassia oil.

Another embodiment comprises cyclosporin A, Lipowax G®, and Cassia oil.

Another embodiment comprises cyclosporin A, Lipowax NI®, and Cassia oil.

Another embodiment comprises cyclosporin A, Lipowax P®, and Cassia oil.

Another embodiment comprises cyclosporin A, Lipowax P-31®, and Cassia oil.

Another embodiment comprises cyclosporin A, Lipowax PR®, and Cassia oil.

Another embodiment comprises cyclosporin A, Lumulse CS-20®, and Cassia oil.

Another embodiment comprises cyclosporin A, Macol CSA-20®, and Cassia oil.

Another embodiment comprises cyclosporin A, Marlox B 24/50®, and Cassia oil.

Another embodiment comprises cyclosporin A, Mazawet 77®, and Cassia oil.

Another embodiment comprises cyclosporin A, Nodox 1713®, and Cassia oil.

Another embodiment comprises cyclosporin A, Norfox 2579®, and Cassia oil.

Another embodiment comprises cyclosporin A, Norfox Lo Foam®, and Cassia oil.

Another embodiment comprises cyclosporin A, Promulgen D®, and Cassia oil.

Another embodiment comprises cyclosporin A, Promulgen G®, and Cassia oil.

Another embodiment comprises cyclosporin A, Renex 30®, and Cassia oil.

Another embodiment comprises cyclosporin A, Renex 36®, and Cassia oil.

Another embodiment comprises cyclosporin A, Rhodasurf A 24®, and Cassia oil.

Another embodiment comprises cyclosporin A, Rhodasurf AAE-10®, and Cassia oil.

Another embodiment comprises cyclosporin A, Rhodasurf BEH-25®, and Cassia oil.

Another embodiment comprises cyclosporin A, Rhodasurf BEH-40®, and Cassia oil.

Another embodiment comprises cyclosporin A, Rhodasurf DA 530®, and Cassia oil.

Another embodiment comprises cyclosporin A, Rhodasurf DA 630®, and Cassia oil.

Another embodiment comprises cyclosporin A, Rhodasurf DA 639®, and Cassia oil.

Another embodiment comprises cyclosporin A, Rhodasurf LAN-23®, and Cassia oil.

Another embodiment comprises cyclosporin A, Rhodasurf ON-870®, and Cassia oil.

Another embodiment comprises cyclosporin A, Rhodasurf ON-877®, and Cassia oil.

Another embodiment comprises cyclosporin A, Rhodasurf TB-970 FLK®, and Cassia oil.

Another embodiment comprises cyclosporin A, Ritacet-20®, and Cassia oil.

Another embodiment comprises cyclosporin A, Ritachol 1000®, and Cassia oil.

Another embodiment comprises cyclosporin A, Ritachol 2000®, and Cassia oil.

Another embodiment comprises cyclosporin A, Ritachol 5000®, and Cassia oil.

Another embodiment comprises cyclosporin A, Ritox 35®, and Cassia oil.

Another embodiment comprises cyclosporin A, Surfonic DA-4®, and Cassia oil.

Another embodiment comprises cyclosporin A, Surfonic DA-6®, and Cassia oil.

Another embodiment comprises cyclosporin A, Surfonic L46-7®, and Cassia oil.

Another embodiment comprises cyclosporin A, Surfonic POA®, and Cassia oil.

Another embodiment comprises cyclosporin A, Synthrapol KB®, and Cassia oil.

Another embodiment comprises cyclosporin A, Teginacid®, and Cassia oil.

Another embodiment comprises cyclosporin A, Teginacid C®, and Cassia oil.

Another embodiment comprises cyclosporin A, Tegotens EC 11®, and Cassia oil.

Another embodiment comprises cyclosporin A, Tinegal NA®, and Cassia oil.

Another embodiment comprises cyclosporin A, Tomadol 400®, and Cassia oil.

Another embodiment comprises cyclosporin A, Tomadol 600®, and Cassia oil.

Another embodiment comprises cyclosporin A, Tomadol 900®, and Cassia oil.

Another embodiment comprises cyclosporin A, Uniperol O®, and Cassia oil.

Another embodiment comprises cyclosporin A, Witconol SN Series®, and Cassia oil.

Another embodiment comprises cyclosporin A, an Ethoxylated Alkylphenol, and Cassia oil.

Another embodiment comprises cyclosporin A, Antarox LF-222®, and Cassia oil.

Another embodiment comprises cyclosporin A, Atlox 775®, and Cassia oil.

Another embodiment comprises cyclosporin A, Caloxylate N-9®, and Cassia oil.

Another embodiment comprises cyclosporin A, Canasol NF-1000®, and Cassia oil.

Another embodiment comprises cyclosporin A, Canasol NF-3000®, and Cassia oil.

Another embodiment comprises cyclosporin A, Canasol NF-3070®, and Cassia oil.

Another embodiment comprises cyclosporin A, Canasol OF 1670®, and Cassia oil.

Another embodiment comprises cyclosporin A, Canasol OF 2570®, and Cassia oil.

Another embodiment comprises cyclosporin A, Canasol OF 4070®, and Cassia oil.

Another embodiment comprises cyclosporin A, Chemax DNP-8®, and Cassia oil.

Another embodiment comprises cyclosporin A, Chemax DNP-18®, and Cassia oil.

Another embodiment comprises cyclosporin A, Chemax DNP-150/50®, and Cassia oil.

Another embodiment comprises cyclosporin A, DeSonic 1.5N®, and Cassia oil.

Another embodiment comprises cyclosporin A, DeSonic 4N®, and Cassia oil.

Another embodiment comprises cyclosporin A, DeSonic 5N®, and Cassia oil.

Another embodiment comprises cyclosporin A, DeSonic 6D®, and Cassia oil.

Another embodiment comprises cyclosporin A, DeSonic 6N®, and Cassia oil.

Another embodiment comprises cyclosporin A, DeSonic 7N®, and Cassia oil.

Another embodiment comprises cyclosporin A, DeSonic 9N®, and Cassia oil.

Another embodiment comprises cyclosporin A, DeSonic 10D®, and Cassia oil.

Another embodiment comprises cyclosporin A, DeSonic 11N®, and Cassia oil.

Another embodiment comprises cyclosporin A, DeSonic 12N®, and Cassia oil.

Another embodiment comprises cyclosporin A, DeSonic 15N®, and Cassia oil.

Another embodiment comprises cyclosporin A, DeSonic 20N®, and Cassia oil.

Another embodiment comprises cyclosporin A, Eccoscour RC®, and Cassia oil.

Another embodiment comprises cyclosporin A, Eccoterge EO-100®, and Cassia oil.

Another embodiment comprises cyclosporin A, Emulsifier 632/90%®, and Cassia oil.

Another embodiment comprises cyclosporin A, Geronol AG-821®, and Cassia oil.

Another embodiment comprises cyclosporin A, Gradonic N-95®, and Cassia oil.

Another embodiment comprises cyclosporin A, Hetoxide NP-4®, and Cassia oil.

Another embodiment comprises cyclosporin A, Hetoxide NP-30®, and Cassia oil.

Another embodiment comprises cyclosporin A, Hostapal N-100®, and Cassia oil.

Another embodiment comprises cyclosporin A, Hostapal N-110®, and Cassia oil.

Another embodiment comprises cyclosporin A, Igepal CTA-639W®, and Cassia oil.

Another embodiment comprises cyclosporin A, Igepal DAP-9®, and Cassia oil.

Another embodiment comprises cyclosporin A, Igepal OD-410®, and Cassia oil.

Another embodiment comprises cyclosporin A, Igepal SS-837®, and Cassia oil.

Another embodiment comprises cyclosporin A, Lipocol NP-9 USP®, and Cassia oil.

Another embodiment comprises cyclosporin A, Macol DNP-10®, and Cassia oil.

Another embodiment comprises cyclosporin A, Marlophen NP 5®, and Cassia oil.

Another embodiment comprises cyclosporin A, Marlophen P 1®, and Cassia oil.

Another embodiment comprises cyclosporin A, Surfonic NB®, and Cassia oil.

Another embodiment comprises cyclosporin A, Surfonic OPB-307®, and Cassia oil.

Another embodiment comprises cyclosporin A, Surfonic OPB-407®, and Cassia oil.

Another embodiment comprises cyclosporin A, Syn Fac 334®, and Cassia oil.

Another embodiment comprises cyclosporin A, Syn Fac 8216®, and Cassia oil.

Another embodiment comprises cyclosporin A, Triton N-57®, and Cassia oil.

Another embodiment comprises cyclosporin A, Trycol 6956®, and Cassia oil.

Another embodiment comprises cyclosporin A, Trycol 6961®, and Cassia oil.

Another embodiment comprises cyclosporin A, Trycol 6964®, and Cassia oil.

Another embodiment comprises cyclosporin A, Trycol 6969®, and Cassia oil.

Another embodiment comprises cyclosporin A, Trycol 6974®, and Cassia oil.

Another embodiment comprises cyclosporin A, Witbreak DRB-127®, and Cassia oil.

Another embodiment comprises cyclosporin A, Witbreak DRB-127®, and Cassia oil.

Another embodiment comprises cyclosporin A, Witconol NP Series®, and Cassia oil.

Another embodiment comprises cyclosporin A, an Ethoxylated Aryl Phenol, and Cassia oil.

Another embodiment comprises cyclosporin A, Soprophor BSU®, and Cassia oil.

Another embodiment comprises cyclosporin A, Soprophor CY/8®, and Cassia oil.

Another embodiment comprises cyclosporin A, Soprophor S/25®, and Cassia oil.

Another embodiment comprises cyclosporin A, Witconol NIO®, and Cassia oil.

Another embodiment comprises cyclosporin A, Witconol NIW®, and Cassia oil.

Another embodiment comprises cyclosporin A, Witconol S-100®, and Cassia oil.

Another embodiment comprises cyclosporin A, an Ethoxylated Fatty Acid, and Cassia oil.

Another embodiment comprises cyclosporin A, Aldo PGHMS®, and Cassia oil.

Another embodiment comprises cyclosporin A, Alkamuls TO-15/HR®, and Cassia oil.

Another embodiment comprises cyclosporin A, Armotan AL-69-66®, and Cassia oil.

Another embodiment comprises cyclosporin A, Cerasynt 840®, and Cassia oil.

Another embodiment comprises cyclosporin A, Cerasynt 945®, and Cassia oil.

Another embodiment comprises cyclosporin A, Crystal Inhibitor No. 5®, and Cassia oil.

Another embodiment comprises cyclosporin A, DeThox Acid L-9®, and Cassia oil.

Another embodiment comprises cyclosporin A, DeThox Acid S-8®, and Cassia oil.

Another embodiment comprises cyclosporin A, Ethofat 242/25®, and Cassia oil.

Another embodiment comprises cyclosporin A, Hydropalat 65®, and Cassia oil.

Another embodiment comprises cyclosporin A, Lipo EGMS®, and Cassia oil.

Another embodiment comprises cyclosporin A, Lipopeg 2 DL®, and Cassia oil.

Another embodiment comprises cyclosporin A, Lipopeg 4 DL®, and Cassia oil.

Another embodiment comprises cyclosporin A, Lipopeg 4-L®, and Cassia oil.

Another embodiment comprises cyclosporin A, Lipopeg 39-S®, and Cassia oil.

Another embodiment comprises cyclosporin A, Lipopeg 4-S®, and Cassia oil.

Another embodiment comprises cyclosporin A, Lipopeg 10-S®, and Cassia oil.

Another embodiment comprises cyclosporin A, Lipopeg 100-S®, and Cassia oil.

Another embodiment comprises cyclosporin A, Lipopeg 6000 DS®, and Cassia oil.

Another embodiment comprises cyclosporin A, Lumulse 40-L®, and Cassia oil.

Another embodiment comprises cyclosporin A, Lumulse 40-S®, and Cassia oil.

Another embodiment comprises cyclosporin A, Lumulse 42-L®, and Cassia oil.

Another embodiment comprises cyclosporin A, Lumulse 42-S®, and Cassia oil.

Another embodiment comprises cyclosporin A, Lumulse 100-S®, and Cassia oil.

Another embodiment comprises cyclosporin A, Lumulse 602-S®, and Cassia oil.

Another embodiment comprises cyclosporin A, Magrabar PGE-20-0®, and Cassia oil.

Another embodiment comprises cyclosporin A, Magrabar PGE-20L®, and Cassia oil.

Another embodiment comprises cyclosporin A, Magrabar PGE-20T®, and Cassia oil.

Another embodiment comprises cyclosporin A, Magrabar PGE-22-0®, and Cassia oil.

Another embodiment comprises cyclosporin A, Magrabar PGE-22L®, and Cassia oil.

Another embodiment comprises cyclosporin A, Magrabar PGE-22T®, and Cassia oil.

Another embodiment comprises cyclosporin A, Magrabar PGE-40-0®, and Cassia oil.

Another embodiment comprises cyclosporin A, Magrabar PGE-40L®, and Cassia oil.

Another embodiment comprises cyclosporin A, Magrabar PGE-40T®, and Cassia oil.

Another embodiment comprises cyclosporin A, Magrabar PGE-42-0®, and Cassia oil.

Another embodiment comprises cyclosporin A, Magrabar PGE-42L®, and Cassia oil.

Another embodiment comprises cyclosporin A, Magrabar PGE-42T®, and Cassia oil.

Another embodiment comprises cyclosporin A, Magrabar PGE-60-0®, and Cassia oil.

Another embodiment comprises cyclosporin A, Magrabar PGE-60L®, and Cassia oil.

Another embodiment comprises cyclosporin A, Magrabar PGE-60T®, and Cassia oil.

Another embodiment comprises cyclosporin A, Magrabar PGE-62-0®, and Cassia oil.

Another embodiment comprises cyclosporin A, Magrabar PGE-62L®, and Cassia oil.

Another embodiment comprises cyclosporin A, Magrabar PGE-62T®, and Cassia oil.

Another embodiment comprises cyclosporin A, Mapeg S-40K®, and Cassia oil.

Another embodiment comprises cyclosporin A, Marlowet OTS®, and Cassia oil.

Another embodiment comprises cyclosporin A, Naturechem PGR®, and Cassia oil.

Another embodiment comprises cyclosporin A, PG No. 4®, and Cassia oil.

Another embodiment comprises cyclosporin A, Renex 20®, and Cassia oil.

Another embodiment comprises cyclosporin A, Ritox 52®, and Cassia oil.

Another embodiment comprises cyclosporin A, Ritox 53®, and Cassia oil.

Another embodiment comprises cyclosporin A, Ritox 59®, and Cassia oil.

Another embodiment comprises cyclosporin A, Surfax 8916/A®, and Cassia oil.

Another embodiment comprises cyclosporin A, Tego Acid S 40 P®, and Cassia oil.

Another embodiment comprises cyclosporin A, Tego Acid S 100 P®, and Cassia oil.

Another embodiment comprises cyciosporin A, Tween 20®, and Cassia oil.

Another embodiment comprises cyclosporin A, Volpo 131®, and Cassia oil.

Another embodiment comprises cyclosporin A, an Ethoxylated Fatty Ester or Oil, and Cassia oil.

Another embodiment comprises cyclosporin A, Acconon 6-C10®, and Cassia oil.

Another embodiment comprises cyclosporin A, Acconon CC-6®, and Cassia oil.

Another embodiment comprises cyclosporin A, Acconon CO-7®, and Cassia oil.

Another embodiment comprises cyclosporin A, Aldosperse 40/60 FG®, and Cassia oil.

Another embodiment comprises cyclosporin A, Aldosperse ML-23®, and Cassia oil.

Another embodiment comprises cyclosporin A, Aldosperse MS-20 FG®, and Cassia oil.

Another embodiment comprises cyclosporin A, Alkamuls EL-620®, and Cassia oil.

Another embodiment comprises cyclosporin A, Alkamuls EL-719®, and Cassia oil.

Another embodiment comprises cyclosporin A, Alkamuls EL-985®, and Cassia oil.

Another embodiment comprises cyclosporin A, Arlatone G®, and Cassia oil.

Another embodiment comprises cyclosporin A, Arlatone T®, and Cassia oil.

Another embodiment comprises cyclosporin A, Atlas G-1045A®, and Cassia oil.

Another embodiment comprises cyclosporin A, Atlas G-1086®, and Cassia oil.

Another embodiment comprises cyclosporin A, Atlas G-1087®, and Cassia oil.

Another embodiment comprises cyclosporin A, Atlas G-1089®, and Cassia oil.

Another embodiment comprises cyclosporin A, Atlas G-1096®, and Cassia oil.

Another embodiment comprises cyclosporin A, Atlas G-1292®, and Cassia oil.

Another embodiment comprises cyclosporin A, Atlas G-1293®, and Cassia oil.

Another embodiment comprises cyclosporin A, Atlas G-1300®, and Cassia oil.

Another embodiment comprises cyclosporin A, Atlas G-7076®, and Cassia oil.

Another embodiment comprises cyclosporin A, Capmul EMG®, and Cassia oil.

Another embodiment comprises cyclosporin A, Chemonic CO-40®, and Cassia oil.

Another embodiment comprises cyclosporin A, Chemonic LI-3®, and Cassia oil.

Another embodiment comprises cyclosporin A, Chemonic LI-7®, and Cassia oil.

Another embodiment comprises cyclosporin A, Cirrasol GM®, and Cassia oil.

Another embodiment comprises cyclosporin A, Cremophor CO 40®, and Cassia oil.

Another embodiment comprises cyclosporin A, Cremophor CO 410®, and Cassia oil.

Another embodiment comprises cyclosporin A, Cremophor EL®, and Cassia oil.

Another embodiment comprises cyclosporin A, Cremophor GC7®, and Cassia oil.

Another embodiment comprises cyclosporin A, Cremophor RH-40®, and Cassia oil.

Another embodiment comprises cyclosporin A, Crovol A-40®, and Cassia oil.

Another embodiment comprises cyclosporin A, Crovol A-70®, and Cassia oil.

Another embodiment comprises cyclosporin A, Crovol M-70®, and Cassia oil.

Another embodiment comprises cyclosporin A, Crovol PK-70®, and Cassia oil.

Another embodiment comprises cyclosporin A, Cutina E-24®, and Cassia oil.

Another embodiment comprises cyclosporin A, Dacospin 12-R®, and Cassia oil.

Another embodiment comprises cyclosporin A, Dehymuls HRE-7®, and Cassia oil.

Another embodiment comprises cyclosporin A, DeSonic 30C®, and Cassia oil.

Another embodiment comprises cyclosporin A, DeSonic 36C®, and Cassia oil.

Another embodiment comprises cyclosporin A, DeSonic 40C®, and Cassia oil.

Another embodiment comprises cyclosporin A, Durfax 60®, and Cassia oil.

Another embodiment comprises cyclosporin A, Durfax 65®, and Cassia oil.

Another embodiment comprises cyclosporin A, Durfax 80®, and Cassia oil.

Another embodiment comprises cyclosporin A, Durfax EOM®, and Cassia oil.

Another embodiment comprises cyclosporin A, Eccoterge NF-2®, and Cassia oil.

Another embodiment comprises cyclosporin A, Emulpon CO-360®, and Cassia oil.

Another embodiment comprises cyclosporin A, Emulpon CO-550®, and Cassia oil.

Another embodiment comprises cyclosporin A, Emulsogen EL®, and Cassia oil.

Another embodiment comprises cyclosporin A, Emulsogen HCO 040®, and Cassia oil.

Another embodiment comprises cyclosporin A, Emulsogen HCO 060®, and Cassia oil.

Another embodiment comprises cyclosporin A, Emulsynt 1055®, and Cassia oil.

Another embodiment comprises cyclosporin A, Ethox 3095®, and Cassia oil.

Another embodiment comprises cyclosporin A, Eumulgin RO-40®, and Cassia oil.

Another embodiment comprises cyclosporin A, Genapol G-260®, and Cassia oil.

Another embodiment comprises cyclosporin A, Glycosperse L-20®, and Cassia oil.

Another embodiment comprises cyclosporin A, Glycosperse O-5®, and Cassia oil.

Another embodiment comprises cyclosporin A, Glycosperse O-20®, and Cassia oil.

Another embodiment comprises cyclosporin A, Glycosperse O-20 FG®, and Cassia oil.

Another embodiment comprises cyclosporin A, Glycosperse S-20®, and Cassia oil.

Another embodiment comprises cyclosporin A, Glycosperse S-20 FG®, and Cassia oil.

Another embodiment comprises cyclosporin A, Glycosperse TS-20®, and Cassia oil.

Another embodiment comprises cyclosporin A, Glycosperse TS-20 FG®, and Cassia oil.

Another embodiment comprises cyclosporin A, Hetan SL®, and Cassia oil.

Another embodiment comprises cyclosporin A, Hetan SO®, and Cassia oil.

Another embodiment comprises cyclosporin A, Hetan SS®, and Cassia oil.

Another embodiment comprises cyclosporin A, Hetoxide C-2®, and Cassia oil.

Another embodiment comprises cyclosporin A, Hetoxide C-9®, and Cassia oil.

Another embodiment comprises cyclosporin A, Hetoxide C-15®, and Cassia oil.

Another embodiment comprises cyclosporin A, Hetoxide C-25®, and Cassia oil.

Another embodiment comprises cyclosporin A, Hetoxide C-40®, and Cassia oil.

Another embodiment comprises cyclosporin A, Hetoxide C-200®, and Cassia oil.

Another embodiment comprises cyclosporin A, Hetoxide C-200-50%®, and Cassia oil.

Another embodiment comprises cyclosporin A, Hetoxide GC-30®, and Cassia oil.

Another embodiment comprises cyclosporin A, Hetoxide HC-60®, and Cassia oil.

Another embodiment comprises cyclosporin A, Ice No. 2®, and Cassia oil.

Another embodiment comprises cyclosporin A, Incrocas 30/40®, and Cassia oil.

Another embodiment comprises cyclosporin A, Lexol EC®, and Cassia oil.

Another embodiment comprises cyclosporin A, Lexol EO®, and Cassia oil.

Another embodiment comprises cyclosporin A, Lipocol HCO-40®, and Cassia oil.

Another embodiment comprises cyclosporin A, Lipocol HCO-60®, and Cassia oil.

Another embodiment comprises cyclosporin A, Lipocol O-3 Special®, and Cassia oil.

Another embodiment comprises cyclosporin A, Lipopeg 2-L®, and Cassia oil.

Another embodiment comprises cyclosporin A, Lipopeg 4-DO®, and Cassia oil.

Another embodiment comprises cyclosporin A, Lipopeg 4-DS®, and Cassia oil.

Another embodiment comprises cyclosporin A, Lipovol GTB®, and Cassia oil.

Another embodiment comprises cyclosporin A, Lonzest SML-20®, and Cassia oil.

Another embodiment comprises cyclosporin A, Lonzest SMO-20®, and Cassia oil.

Another embodiment comprises cyclosporin A, Lonzest SMS-20®, and Cassia oil.

Another embodiment comprises cyclosporin A, Lonzest STO-20®, and Cassia oil.

Another embodiment comprises cyclosporin A, Lonzest STS-20®, and Cassia oil.

Another embodiment comprises cyclosporin A, Lumulse GR-40®, and Cassia oil.

Another embodiment comprises cyclosporin A, Lumulse GRH-40®, and Cassia oil.

Another embodiment comprises cyclosporin A, Lumulse POE (7) GML®, and Cassia oil.

Another embodiment comprises cyclosporin A, Lumulse POE (12) Glyc®, and Cassia oil.

Another embodiment comprises cyclosporin A, Lumulse POE (40) MS KF®, and Cassia oil.

Another embodiment comprises cyclosporin A, Marlowet 4750®, and Cassia oil.

Another embodiment comprises cyclosporin A, Marlowet LVS®, and Cassia oil.

Another embodiment comprises cyclosporin A, Marlowet R 11®, and Cassia oil.

Another embodiment comprises cyclosporin A, Marlowet R 40®, and Cassia oil.

Another embodiment comprises cyclosporin A, Mazol 80 MGK®, and Cassia oil.

Another embodiment comprises cyclosporin A, Nonionic Emulsifier T-9®, and Cassia oil.

Another embodiment comprises cyclosporin A, Oronal LCG®, and Cassia oil.

Another embodiment comprises cyclosporin A, Polyderm PPI-CO-200®, and Cassia oil.

Another embodiment comprises cyclosporin A, Polyderm PPI-CO-40®, and Cassia oil.

Another embodiment comprises cyclosporin A, Rewoderm LI 520-70®, and Cassia oil.

Another embodiment comprises cyclosporin A, Ritapeg 150 DS®, and Cassia oil.

Another embodiment comprises cyclosporin A, Softigen 767®, and Cassia oil.

Another embodiment comprises cyclosporin A, Surfactol 318®, and Cassia oil.

Another embodiment comprises cyclosporin A, Surfactol 365®, and Cassia oil.

Another embodiment comprises cyclosporin A, Syn Lube 107®, and Cassia oil.

Another embodiment comprises cyclosporin A, Syn Lube 728®, and Cassia oil.

Another embodiment comprises cyclosporin A, Syn Lube 1632H®, and Cassia oil.

Another embodiment comprises cyclosporin A, Syn Lube 6277-A®, and Cassia oil.

Another embodiment comprises cyclosporin A, T-Det C-20®, and Cassia oil.

Another embodiment comprises cyclosporin A, T-Det C-40®, and Cassia oil.

Another embodiment comprises cyclosporin A, Tally 100 Plus®, and Cassia oil.

Another embodiment comprises cyclosporin A, Uniperol EL®, and Cassia oil.

Another embodiment comprises cyclosporin A, a Fatty Ester, and Cassia oil.

Another embodiment comprises cyclosporin A, Actralube-Syn 147®, and Cassia oil.

Another embodiment comprises cyclosporin A, Atlas G-1556®, and Cassia oil.

Another embodiment comprises cyclosporin A, Atlas G-1564®, and Cassia oil.

Another embodiment comprises cyclosporin A, Atlasol Base Oil S®, and Cassia oil.

Another embodiment comprises cyclosporin A, Base ML®, and Cassia oil.

Another embodiment comprises cyclosporin A, Base MT®, and Cassia oil.

Another embodiment comprises cyclosporin A, Cerasynt 303®, and Cassia oil.

Another embodiment comprises cyclosporin A, Dermol 1012®, and Cassia oil.

Another embodiment comprises cyclosporin A, Kemester 4000®, and Cassia oil.

Another embodiment comprises cyclosporin A, Lactipol S®, and Cassia oil.

Another embodiment comprises cyclosporin A, Magrabar PGO®, and Cassia oil.

Another embodiment comprises cyclosporin A, Mayco Base BFO®, and Cassia oil.

Another embodiment comprises cyclosporin A, Methyl Linoleate®, and Cassia oil.

Another embodiment comprises cyclosporin A, Pationic 122A®, and Cassia oil.

Another embodiment comprises cyclosporin A, Pationic 138C®, and Cassia oil.

Another embodiment comprises cyclosporin A, Pationic CSL®, and Cassia oil.

Another embodiment comprises cyclosporin A, Pationic ISL®, and Cassia oil.

Another embodiment comprises cyclosporin A, Pationic SBL®, and Cassia oil.

Another embodiment comprises cyclosporin A, Pationic SSL®, and Cassia oil.

Another embodiment comprises cyclosporin A, Ritasol®, and Cassia oil.

Another embodiment comprises cyclosporin A, Tego Alkanol CS 20®, and Cassia oil.

Another embodiment comprises cyclosporin A, Tego Alkanol L23 P® and Cassia oil.

Another embodiment comprises cyclosporin A, Tego Alkanol S2®, and Cassia oil.

Another embodiment comprises cyclosporin A, Tego Alkanol S20 P®, and Cassia oil.

Another embodiment comprises cyclosporin A, Triemulsifier 600 MS®, and Cassia oil.

Another embodiment comprises cyclosporin A, a Glycerol Esters, and Cassia oil.

Another embodiment comprises cyclosporin A, Agro #9 Wint SBO®, and Cassia oil.

Another embodiment comprises cyclosporin A, Ahcovel Base 700®, and Cassia oil.

Another embodiment comprises cyclosporin A, Aldo HMS FG®, and Cassia oil.

Another embodiment comprises cyclosporin A, Aldo MLD®, and Cassia oil.

Another embodiment comprises cyclosporin A, Aldo MLD FG®, and Cassia oil.

Another embodiment comprises cyclosporin A, Aldo MO FG®, and Cassia oil.

Another embodiment comprises cyclosporin A, Aldo MS®, and Cassia oil.

Another embodiment comprises cyclosporin A, Aldo MS FG®, and Cassia oil.

Another embodiment comprises cyclosporin A, Aldo MS LG FG®, and Cassia oil.

Another embodiment comprises cyclosporin A, Aldo MSD®, and Cassia oil.

Another embodiment comprises cyclosporin A, Aldo MSD FG®, and Cassia oil.

Another embodiment comprises cyclosporin A, Aldosperse O-20 FG®, and Cassia oil.

Another embodiment comprises cyclosporin A, Aldosperse TS-20 FG®, and Cassia oil.

Another embodiment comprises cyclosporin A, Aldosperse TS-40 FG®, and Cassia oil.

Another embodiment comprises cyclosporin A, Arlacel 165®, and Cassia oil.

Another embodiment comprises cyclosporin A, Arlacel 186®, and Cassia oil.

Another embodiment comprises cyclosporin A, Capmul GMO®, and Cassia oil.

Another embodiment comprises cyclosporin A, Capmul GMS®, and Cassia oil.

Another embodiment comprises cyclosporin A, Caprol 3GO®, and Cassia oil.

Another embodiment comprises cyclosporin A, Caprol 3GVS®, and Cassia oil.

Another embodiment comprises cyclosporin A, Caprol 6G2S®, and Cassia oil.

Another embodiment comprises cyclosporin A, Caprol 10G40®, and Cassia oil.

Another embodiment comprises cyclosporin A, Caprol 10G100®, and Cassia oil.

Another embodiment comprises cyclosporin A, Caprol ET®, and Cassia oil.

Another embodiment comprises cyclosporin A, Caprol PGE860®, and Cassia oil.

Another embodiment comprises cyclosporin A, Cerasynt 945®, and Cassia oil.

Another embodiment comprises cyclosporin A, Cerasynt GMS®, and Cassia oil.

Another embodiment comprises cyclosporin A, Cerasynt Q®, and Cassia oil.

Another embodiment comprises cyclosporin A, Cerasynt SD®, and Cassia oil.

Another embodiment comprises cyclosporin A, Cerasynt WM®, and Cassia oil.

Another embodiment comprises cyclosporin A, Chemsperse 14®, and Cassia oil.

Another embodiment comprises cyclosporin A, Cremophor GO-32®, and Cassia oil.

Another embodiment comprises cyclosporin A, Cremophor GS11®, and Cassia oil.

Another embodiment comprises cyclosporin A, Cremophor GS-32®, and Cassia oil.

Another embodiment comprises cyclosporin A, Cutina KD-16®, and Cassia oil.

Another embodiment comprises cyclosporin A, Dehymuls PGPH®, and Cassia oil.

Another embodiment comprises cyclosporin A, Dermol DGDIS®, and Cassia oil.

Another embodiment comprises cyclosporin A, Dermol DGMIS®, and Cassia oil.

Another embodiment comprises cyclosporin A, Dermol G-76®, and Cassia oil.

Another embodiment comprises cyclosporin A, Dermol G-7DI®, and Cassia oil.

Another embodiment comprises cyclosporin A, Dermol NGDI®, and Cassia oil.

Another embodiment comprises cyclosporin A, Dermolan GLH®, and Cassia oil.

Another embodiment comprises cyclosporin A, Drewmulse GMO®, and Cassia oil.

Another embodiment comprises cyclosporin A, Drewpol 3-5-M®, and Cassia oil.

Another embodiment comprises cyclosporin A, Durlac 100 W®, and Cassia oil.

Another embodiment comprises cyclosporin A, Dur-Lo®, and Cassia oil.

Another embodiment comprises cyclosporin A, Dynasan 118®, and Cassia oil.

Another embodiment comprises cyclosporin A, EC-25®, and Cassia oil.

Another embodiment comprises cyclosporin A, EM 40®, and Cassia oil.

Another embodiment comprises cyclosporin A, Emerest 2400®, and Cassia oil.

Another embodiment comprises cyclosporin A, Emerest 2452®, and Cassia oil.

Another embodiment comprises cyclosporin A, Empilan G-26®, and Cassia oil.

Another embodiment comprises cyclosporin A, Genapol TSM®, and Cassia oil.

Another embodiment comprises cyclosporin A, Hostacerin DGI®, and Cassia oil.

Another embodiment comprises cyclosporin A, Hostacerin DGL®, and Cassia oil.

Another embodiment comprises cyclosporin A, Hostacerin DGMS®, and Cassia oil.

Another embodiment comprises cyclosporin A, Hostacerin DGSB®, and Cassia oil.

Another embodiment comprises cyclosporin A, Ice No. 2®, and Cassia oil.

Another embodiment comprises cyclosporin A, Imwitor 742®, and Cassia oil.

Another embodiment comprises cyclosporin A, Imwitor 780 K®, and Cassia oil.

Another embodiment comprises cyclosporin A, Imwitor 960 Flakes®, and Cassia oil.

Another embodiment comprises cyclosporin A, Isolan GI 34®, and Cassia oil.

Another embodiment comprises cyclosporin A, Isolan GO 33®, and Cassia oil.

Another embodiment comprises cyclosporin A, Kemester 1000®, and Cassia oil.

Another embodiment comprises cyclosporin A, Kemester 2000®, and Cassia oil.

Another embodiment comprises cyclosporin A, Kemester 2000®, and Cassia oil.

Another embodiment comprises cyclosporin A, Kemester 6000SE®, and Cassia oil.

Another embodiment comprises cyclosporin A, Lamecreme DGE 18®, and Cassia oil.

Another embodiment comprises cyclosporin A, Lexemul 515®, and Cassia oil.

Another embodiment comprises cyclosporin A, Lexemul 561®, and Cassia oil.

Another embodiment comprises cyclosporin A, Lexemul AR®, and Cassia oil.

Another embodiment comprises cyclosporin A, Lexemul AS®, and Cassia oil.

Another embodiment comprises cyclosporin A, Lexemul GDL®, and Cassia oil.

Another embodiment comprises cyclosporin A, Lexemul T®, and Cassia oil.

Another embodiment comprises cyclosporin A, Lipomulse 165®, and Cassia oil.

Another embodiment comprises cyclosporin A, Lumulse GML K®, and Cassia oil.

Another embodiment comprises cyclosporin A, Lumulse GMO K®, and Cassia oil.

Another embodiment comprises cyclosporin A, Lumulse GMR K®, and Cassia oil.

Another embodiment comprises cyclosporin A, Lumulse GMT K®, and Cassia oil.

Another embodiment comprises cyclosporin A, Magrabar GMC®, and Cassia oil.

Another embodiment comprises cyclosporin A, Magrabar GMO-CK®, and Cassia oil.

Another embodiment comprises cyclosporin A, Magrabar MDG-5050®, and Cassia oil.

Another embodiment comprises cyclosporin A, Magrabar PGO-315®, and Cassia oil.

Another embodiment comprises cyclosporin A, Magrabar PGO-1010®, and Cassia oil.

Another embodiment comprises cyclosporin A, Mazol 300K®, and Cassia oil.

Another embodiment comprises cyclosporin A, Mazol GMO-K®, and Cassia oil.

Another embodiment comprises cyclosporin A, Mazol GMS-K®, and Cassia oil.

Another embodiment comprises cyclosporin A, Mazol PG031-K®, and Cassia oil.

Another embodiment comprises cyclosporin A, Miglyol 812®, and Cassia oil.

Another embodiment comprises cyclosporin A, Norfox 165C®, and Cassia oil.

Another embodiment comprises cyclosporin A, Scherdemol GMIS®, and Cassia oil.

Another embodiment comprises cyclosporin A, Tegin®, and Cassia oil.

Another embodiment comprises cyclosporin A, Tegin 4100 Pellets®, and Cassia oil.

Another embodiment comprises cyclosporin A, Tegin M Pellets®, and Cassia oil.

Another embodiment comprises cyclosporin A, Tegin OV®, and Cassia oil.

Another embodiment comprises cyclosporin A, Teginacid H®, and Cassia oil.

Another embodiment comprises cyclosporin A, Tego Cosmo P813®, and Cassia oil.

Another embodiment comprises cyclosporin A, Wickenol 535®, and Cassia oil.

Another embodiment comprises cyclosporin A, Witconol 14®, and Cassia oil.

Another embodiment comprises cyclosporin A, Witconol 14®, and Cassia oil.

Another embodiment comprises cyclosporin A, Witconol 14F®, and Cassia oil.

Another embodiment comprises cyclosporin A, Witconol 18L®, and Cassia oil.

Another embodiment comprises cyclosporin A, Witconol GOT®, and Cassia oil.

Another embodiment comprises cyclosporin A, Witconol MST®, and Cassia oil.

Another embodiment comprises cyclosporin A, Witconol RHT®, and Cassia oil.

Another embodiment comprises cyclosporin A, a Glycol Ester, and Cassia oil.

Another embodiment comprises cyclosporin A, Alkamuls 600 DO®, and Cassia oil.

Another embodiment comprises cyclosporin A, Alkamuls SEG®, and Cassia oil.

Another embodiment comprises cyclosporin A, Atlas EM-2®, and Cassia oil.

Another embodiment comprises cyclosporin A, Cerasynt IP®, and Cassia oil.

Another embodiment comprises cyclosporin A, Cerasynt M®, and Cassia oil.

Another embodiment comprises cyclosporin A, Cerasynt MN®, and Cassia oil.

Another embodiment comprises cyclosporin A, Cerasynt PA®, and Cassia oil.

Another embodiment comprises cyclosporin A, Chemsperse EGDS®, and Cassia oil.

Another embodiment comprises cyclosporin A, Chemsperse EGMS®, and Cassia oil.

Another embodiment comprises cyclosporin A, Colonial Monolaurin®, and Cassia oil.

Another embodiment comprises cyclosporin A, DeMuls SGE-95®, and Cassia oil.

Another embodiment comprises cyclosporin A, Eccoterge 200®, and Cassia oil.

Another embodiment comprises cyclosporin A, Emerest 2380®, and Cassia oil.

Another embodiment comprises cyclosporin A, Ethox 2610®, and Cassia oil.

Another embodiment comprises cyclosporin A, Ethox DO-9®, and Cassia oil.

Another embodiment comprises cyclosporin A, Ethox DO-14®, and Cassia oil.

Another embodiment comprises cyclosporin A, Ethox SO-9®, and Cassia oil.

Another embodiment comprises cyclosporin A, Fizul MD-318®, and Cassia oil.

Another embodiment comprises cyclosporin A, Genapol EGDS-VHP®, and Cassia oil.

Another embodiment comprises cyclosporin A, Genapol TS Powder®, and Cassia oil.

Another embodiment comprises cyclosporin A, Hostacerin WO®, and Cassia oil.

Another embodiment comprises cyclosporin A, Inversol 140®, and Cassia oil.

Another embodiment comprises cyclosporin A, Kemester 104®, and Cassia oil.

Another embodiment comprises cyclosporin A, Kemester 205®, and Cassia oil.

Another embodiment comprises cyclosporin A, Kemester 226®, and Cassia oil.

Another embodiment comprises cyclosporin A, Kemester 5221SE®, and Cassia oil.

Another embodiment comprises cyclosporin A, Kemester EGDS®, and Cassia oil.

Another embodiment comprises cyclosporin A, Lexemul EGDS®, and Cassia oil.

Another embodiment comprises cyclosporin A, Lexemul EGMS®, and Cassia oil.

Another embodiment comprises cyclosporin A, Lexemul P® and Cassia oil.

Another embodiment comprises cyclosporin A, Lipo DGLS®, and Cassia oil.

Another embodiment comprises cyclosporin A, Lipo EGDS®, and Cassia oil.

Another embodiment comprises cyclosporin A, Lipo PGMS®, and Cassia oil.

Another embodiment comprises cyclosporin A, Liposorb S-4®, and Cassia oil.

Another embodiment comprises cyclosporin A, Liposorb TO-20®, and Cassia oil.

Another embodiment comprises cyclosporin A, Lumulse PGO®, and Cassia oil.

Another embodiment comprises cyclosporin A, Mackester EGDS®, and Cassia oil.

Another embodiment comprises cyclosporin A, Mackester EGMS®, and Cassia oil.

Another embodiment comprises cyclosporin A, Mackester GSTP®, and Cassia oil.

Another embodiment comprises cyclosporin A, Mackester Series®, and Cassia oil.

Another embodiment comprises cyclosporin A, Magrabar PDG-50®, and Cassia oil.

Another embodiment comprises cyclosporin A, Mapeg 6000 DS®, and Cassia oil.

Another embodiment comprises cyclosporin A, Marlowet 4702®, and Cassia oil.

Another embodiment comprises cyclosporin A, Monalube 305®, and Cassia.oil.

Another embodiment comprises cyclosporin A, Monalube 310®, and Cassia oil.

Another embodiment comprises cyclosporin A, Monalube 315®, and Cassia oil.

Another embodiment comprises cyclosporin A, Monalube 320®, and Cassia oil.

Another embodiment comprises cyclosporin A, Monalube 325®, and Cassia oil.

Another embodiment comprises cyclosporin A, Monalube 330®, and Cassia oil.

Another embodiment comprises cyclosporin A, Naturechem PGHS®, and Cassia oil.

Another embodiment comprises cyclosporin A, Polycastorol PLO-840®, and Cassia oil.

Another embodiment comprises cyclosporin A, Polytex 10M®, and Cassia oil.

Another embodiment comprises cyclosporin A, Ritasynt IP®, and Cassia oil.

Another embodiment comprises cyclosporin A, Ross Chem PEG 600 DT®, and Cassia oil.

Another embodiment comprises cyclosporin A, Schercemol PGMS®, and Cassia oil.

Another embodiment comprises cyclosporin A, Sponto H-44C®, and Cassia oil.

Another embodiment comprises cyclosporin A, Tegin G®, and Cassia oil.

Another embodiment comprises cyclosporin A, Witbreak DGE-182®, and Cassia oil.

Another embodiment comprises cyclosporin A, Witbreak DGE-182®, and Cassia oil.

Another embodiment comprises cyclosporin A, Witbreak DRA-21®, and Cassia oil.

Another embodiment comprises cyclosporin A, Witbreak DRA-21®, and Cassia oil.

Another embodiment comprises cyclosporin A, Witbreak DRA-50®, and Cassia oil.

Another embodiment comprises cyclosporin A, Witbreak DRA-50®, and Cassia oil.

Another embodiment comprises cyclosporin A, Witconol F26-46®, and Cassia oil.

Another embodiment comprises cyclosporin A, Witconol H-32®, and Cassia oil.

Another embodiment comprises cyclosporin A, Witconol H-33®, and Cassia oil.

Another embodiment comprises cyclosporin A, Witconol H-35A®, and Cassia oil.

Another embodiment comprises cyclosporin A, Witconol RHP®, and Cassia oil.

Another embodiment comprises cyclosporin A, a Lanolin-based Derivative, and Cassia oil.

Another embodiment comprises cyclosporin A, Amerchol CAB®, and Cassia oil.

Another embodiment comprises cyclosporin A, Amerchol L-101®, and Cassia oil.

Another embodiment comprises cyclosporin A, Amerlate LFA-LO®, and Cassia oil.

Another embodiment comprises cyclosporin A, Amerlate P® and Cassia oil.

Another embodiment comprises cyclosporin A, Barre Common Degras®, and Cassia oil.

Another embodiment comprises cyclosporin A, Cholesterol NF®, and Cassia oil.

Another embodiment comprises cyclosporin A, Crodalan AWS®, and Cassia oil.

Another embodiment comprises cyclosporin A, Crodalan LA®, and Cassia oil.

Another embodiment comprises cyclosporin A, Emery 1650®, and Cassia oil.

Another embodiment comprises cyclosporin A, Emery 1650®, and Cassia oil.

Another embodiment comprises cyclosporin A, Emery 1740®, and Cassia oil.

Another embodiment comprises cyclosporin A, Emery 1740®, and Cassia oil.

Another embodiment comprises cyclosporin A, Forlan 500®, and Cassia oil.

Another embodiment comprises cyclosporin A, Forlan L®, and Cassia oil.

Another embodiment comprises cyclosporin A, Laneto 50®, and Cassia oil.

Another embodiment comprises cyclosporin A, Laneto 100®, and Cassia oil.

Another embodiment comprises cyclosporin A, Laneto AWS®, and Cassia oil.

Another embodiment comprises cyclosporin A, Lanfrax 1776®, and Cassia oil.

Another embodiment comprises cyclosporin A, Lanfrax 1776®, and Cassia oil.

Another embodiment comprises cyclosporin A, Lanogel 21®, and Cassia oil.

Another embodiment comprises cyclosporin A, Lipolan®, and Cassia oil.

Another embodiment comprises cyclosporin A, Lipolan 31®, and Cassia oil.

Another embodiment comprises cyclosporin A, OHlan®, and Cassia oil.

Another embodiment comprises cyclosporin A, Polychol 5®, and Cassia oil.

Another embodiment comprises cyclosporin A, Polychol 15®, and Cassia oil.

Another embodiment comprises cyclosporin A, Ritacetyl®, and Cassia oil.

Another embodiment comprises cyclosporin A, Ritachol®, and Cassia oil.

Another embodiment comprises cyclosporin A, Ritahydrox®, and Cassia oil.

Another embodiment comprises cyclosporin A, Ritalafa®, and Cassia oil.

Another embodiment comprises cyclosporin A, Ritalan®, and Cassia oil.

Another embodiment comprises cyclosporin A, Ritalan AWS®, and Cassia oil.

Another embodiment comprises cyclosporin A, Ritalan C®, and Cassia oil.

Another embodiment comprises cyclosporin A, Ritawax®, and Cassia oil.

Another embodiment comprises cyclosporin A, Ritawax AEO®, and Cassia oil.

Another embodiment comprises cyclosporin A, Ritawax ALA®, and Cassia oil.

Another embodiment comprises cyclosporin A, Solan/Solan 50/Super Solan®, and Cassia oil.

Another embodiment comprises cyclosporin A, Super Hartolan/Hartolan®, and Cassia oil.

Another embodiment comprises cyclosporin A, Supersat AWS-4®, and Cassia oil.

Another embodiment comprises cyclosporin A, Supersat AWS-24®, and Cassia oil.

Another embodiment comprises cyclosporin A, a Lecithin or Lecithin Derivative, and Cassia oil.

Another embodiment comprises cyclosporin A, Alcolec®, and Cassia oil.

Another embodiment comprises cyclosporin A, Lecithin®, and Cassia oil.

Another embodiment comprises cyclosporin A, Lexin K®, and Cassia oil.

Another embodiment comprises cyclosporin A, Natipide®, and Cassia oil.

Another embodiment comprises cyclosporin A, a Lignin or Lignin Derivative, and Cassia oil.

Another embodiment comprises cyclosporin A, Diwatex XP 9®, and Cassia oil.

Another embodiment comprises cyclosporin A, Dynasperse LCD®, and Cassia oil.

Another embodiment comprises cyclosporin A, Indulin SAL®, and Cassia oil.

Another embodiment comprises cyclosporin A, Indulin W-1®, and Cassia oil.

Another embodiment comprises cyclosporin A, Indulin W-5®, and Cassia oil.

Another embodiment comprises cyclosporin A, Lignosol FTA®, and Cassia oil.

Another embodiment comprises cyclosporin A, Lignosol SFX-65®, and Cassia oil.

Another embodiment comprises cyclosporin A, Marasperse 52 CP®, and Cassia oil.

Another embodiment comprises cyclosporin A, Marasperse AG®, and Cassia oil.

Another embodiment comprises cyclosporin A, Marasperse CBOS-4®, and Cassia oil.

Another embodiment comprises cyclosporin A, Ufoxane 2®, and Cassia oil.

Another embodiment comprises cyclosporin A, a Methyl Ester, and Cassia oil.

Another embodiment comprises cyclosporin A, E.B. Cleaner AK®, and Cassia oil.

Another embodiment comprises cyclosporin A, Oleocal ME-70®, and Cassia oil.

Another embodiment comprises cyclosporin A, Trade Name Company®, and Cassia oil.

Another embodiment comprises cyclosporin A, Methyl Esters (cont'd)®, and Cassia oil.

Another embodiment comprises cyclosporin A, Oleocal ME-92®, and Cassia oil.

Another embodiment comprises cyclosporin A, Oleocal ME-112®, and Cassia oil.

Another embodiment comprises cyclosporin A, Oleocal ME-130®, and Cassia oil.

Another embodiment comprises cyclosporin A, a Monoglyceride or a Derivative thereof, and Cassia oil.

Another embodiment comprises cyclosporin A, Dynacet 211®, and Cassia oil.

Another embodiment comprises cyclosporin A, Hetsorb S-20®, and Cassia oil.

Another embodiment comprises cyclosporin A, Imwitor 191®, and Cassia oil.

Another embodiment comprises cyclosporin A, Imwitor 370®, and Cassia oil.

Another embodiment comprises cyclosporin A, Imwitor 375®, and Cassia oil.

Another embodiment comprises cyclosporin A, Imwitor 900®, and Cassia oil.

Another embodiment comprises cyclosporin A, Imwitor 945®, and Cassia oil.

Another embodiment comprises cyclosporin A, Imwitor 2020®, and Cassia oil.

Another embodiment comprises cyclosporin A, Kemester 5500®, and Cassia oil.

Another embodiment comprises cyclosporin A, Kemester 6000®, and Cassia oil.

Another embodiment comprises cyclosporin A, Magrabar GMC®, and Cassia oil.

Another embodiment comprises cyclosporin A, Magrabar GMO-CK®, and Cassia oil.

Another embodiment comprises cyclosporin A, Magrabar GPC-10®, and Cassia oil.

Another embodiment comprises cyclosporin A, Magrabar MDG-5050®, and Cassia oil.

Another embodiment comprises cyclosporin A, Monalube 335®, and Cassia oil.

Another embodiment comprises cyclosporin A, Monoglycerides Glyceryl Monestearate Archer Daniels Midland Company®, and Cassia oil.

Another embodiment comprises cyclosporin A, Rita GMS®, and Cassia oil.

Another embodiment comprises cyclosporin A, RitamdIse SCG®, and Cassia oil.

Another embodiment comprises cyclosporin A, Softigen 701®, and Cassia oil.

Another embodiment comprises cyclosporin A, Tally 100 Plus®, and Cassia oil.

Another embodiment comprises cyclosporin A, a Polyethylene Glycol, and Cassia oil.

Another embodiment comprises cyclosporin A, Emulgade PL 68/50®, and Cassia oil.

Another embodiment comprises cyclosporin A, Lumulse PEG®, and Cassia oil.

Another embodiment comprises cyclosporin A, Rhodasurf PEG-400®, and Cassia oil.

Another embodiment comprises cyclosporin A, Rhodasurf PEG-600®, and Cassia oil.

Another embodiment comprises cyclosporin A, Witconol PEG-400®, and Cassia oil.

Another embodiment comprises cyclosporin A, a Polymeric Surfactant, and Cassia oil.

Another embodiment comprises cyclosporin A, Acritamer PNC-EG®, and Cassia oil.

Another embodiment comprises cyclosporin A, Ag-Rho DEP-775®, and Cassia oil.

Another embodiment comprises cyclosporin A, APG 325N Glycoside®, and Cassia oil.

Another embodiment comprises cyclosporin A, Aristoflex AVC®, and Cassia oil.

Another embodiment comprises cyclosporin A, Aristoflex HMB®, and Cassia oil.

Another embodiment comprises cyclosporin A, Burco NPS-225®, and Cassia oil.

Another embodiment comprises cyclosporin A, Burco NPS-816®, and Cassia oil.

Another embodiment comprises cyclosporin A, Chemccinate 5603®, and Cassia oil.

Another embodiment comprises cyclosporin A, Cosmedia Guar C-261 N®, and Cassia oil.

Another embodiment comprises cyclosporin A, Gantrez S-95®, and Cassia oil.

Another embodiment comprises cyclosporin A, Glucopon 220 UP®, and Cassia oil.

Another embodiment comprises cyclosporin A, Glucopon 225 DK®, and Cassia oil.

Another embodiment comprises cyclosporin A, Glucopon 425 N®, and Cassia oil.

Another embodiment comprises cyclosporin A, Glucopon 600 UP®, and Cassia oil.

Another embodiment comprises cyclosporin A, Glucopon 625 UP®, and Cassia oil.

Another embodiment comprises cyclosporin A, Pemulen 1621®, and Cassia oil.

Another embodiment comprises cyclosporin A, Pemulen 1622®, and Cassia oil.

Another embodiment comprises cyclosporin A, Pemulen TR-1®, and Cassia oil.

Another embodiment comprises cyclosporin A, Pemulen TR-2®, and Cassia oil.

Another embodiment comprises cyclosporin A, Plantacare 818®, and Cassia oil.

Another embodiment comprises cyclosporin A, Plantapon LGC Sorb®, and Cassia oil.

Another embodiment comprises cyclosporin A, Plantaren 1200N®, and Cassia oil.

Another embodiment comprises cyclosporin A, Plantaren 2000N®, and Cassia oil.

Another embodiment comprises cyclosporin A, Viscolam AT 64®, and Cassia oil.

Another embodiment comprises cyclosporin A, Viscolam AT 64P®, and Cassia oil.

Another embodiment comprises cyclosporin A, Viscolam AT 100®, and Cassia oil.

Another embodiment comprises cyclosporin A, Viscolam MAC 7®, and Cassia oil.

Another embodiment comprises cyclosporin A, Viscolam SMC 20®, and Cassia oil.

Another embodiment comprises cyclosporin A, Witbreak RTC-323®, and Cassia oil.

Another embodiment comprises cyclosporin A, WSI 3700®, and Cassia oil.

Another embodiment comprises cyclosporin A, a Propoxylated or Ethoxylated Fatty Acid, Alcohol, or Alkyl Phenol, and Cassia oil.

Another embodiment comprises cyclosporin A, Antarox AA-60®, and Cassia oil.

Another embodiment comprises cyclosporin A, Antarox LF-224®, and Cassia oil.

Another embodiment comprises cyclosporin A, Burcomul DFE-45®, and Cassia oil.

Another embodiment comprises cyclosporin A, Burcoterge LFE-1000®, and Cassia oil.

Another embodiment comprises cyclosporin A, Chemal LF-25B®, and Cassia oil.

Another embodiment comprises cyclosporin A, Chemal LF-40B®, and Cassia oil.

Another embodiment comprises cyclosporin A, Dehypon LS-36®, and Cassia oil.

Another embodiment comprises cyclosporin A, Dehypon LS-36®, and Cassia oil.

Another embodiment comprises cyclosporin A, Dehypon LS-54®, and Cassia oil.

Another embodiment comprises cyclosporin A, Dehypon LS-54®, and Cassia oil.

Another embodiment comprises cyclosporin A, Delonic 100 VLF®, and Cassia oil.

Another embodiment comprises cyclosporin A, Delonic LF-60 MOD®, and Cassia oil.

Another embodiment comprises cyclosporin A, Empiderm B®, and Cassia oil.

Another embodiment comprises cyclosporin A, Ethylan 1206®, and Cassia oil.

Another embodiment comprises cyclosporin A, Ethylan NS-500K®, and Cassia oil.

Another embodiment comprises cyclosporin A, Ethylan NS-500Lq®, and Cassia oil.

Another embodiment comprises cyclosporin A, Genapol 1392®, and Cassia oil.

Another embodiment comprises cyclosporin A, Genapol 2317®, and Cassia oil.

Another embodiment comprises cyclosporin A, Genapol 26EP710®, and Cassia oil.

Another embodiment comprises cyclosporin A, Genapol EP 1022®, and Cassia oil.

Another embodiment comprises cyclosporin A, Genapol EP 1024®, and Cassia oil.

Another embodiment comprises cyclosporin A, Genapol EP 6068®, and Cassia oil.

Another embodiment comprises cyclosporin A, Genapol NP915®, and Cassia oil.

Another embodiment comprises cyclosporin A, Kieralon MFB®, and Cassia oil.

Another embodiment comprises cyclosporin A, Lumisolve CSA-80 V®, and Cassia oil.

Another embodiment comprises cyclosporin A, Marlowet 5001®, and Cassia oil.

Another embodiment comprises cyclosporin A, Marlox FK 64®, and Cassia oil.

Another embodiment comprises cyclosporin A, Marlox MO 124®, and Cassia oil.

Another embodiment comprises cyclosporin A, Marlox S 58®, and Cassia oil.

Another embodiment comprises cyclosporin A, Nonatell 1003®, and Cassia oil.

Another embodiment comprises cyclosporin A, Nonatell 1038®, and Cassia oil.

Another embodiment comprises cyclosporin A, Nonatell 1052, and Cassia oil.

Another embodiment comprises cyclosporin A, Nonatell 1061®, and Cassia oil.

Another embodiment comprises cyclosporin A, Nonatell 1075®, and Cassia oil.

Another embodiment comprises cyclosporin A, Nonatell 1088®, and Cassia oil.

Another embodiment comprises cyclosporin A, Nonatell 1123®, and Cassia oil.

Another embodiment comprises cyclosporin A, Nonatell 1153®, and Cassia oil.

Another embodiment comprises cyclosporin A, Nonatell 1161®, and Cassia oil.

Another embodiment comprises cyclosporin A, Nonatell 1172, and Cassia oil.

Another embodiment comprises cyclosporin A, Nonatell 1181®, and Cassia oil.

Another embodiment comprises cyclosporin A, Norfox 36®, and Cassia oil.

Another embodiment comprises cyclosporin A, Procetyl AWS®, and Cassia oil.

Another embodiment comprises cyclosporin A, Sandoiylate SX 412®, and Cassia oil.

Another embodiment comprises cyclosporin A, Sandoxylate SX 418®, and Cassia oil.

Another embodiment comprises cyclosporin A, Surfonic JL-80X®, and Cassia oil.

Another embodiment comprises cyclosporin A, Sunfonic JL-80X-B1®, and Cassia oil.

Another embodiment comprises cyclosporin A, Surfonic L4-29X®, and Cassia oil.

Another embodiment comprises cyclosporin A, Sunonic LF®, and Cassia oil.

Another embodiment comprises cyclosporin A, T-Det A826®, and Cassia oil.

Another embodiment comprises cyclosporin A, T-Det LF-416®, and Cassia oil.

Another embodiment comprises cyclosporin A, Tergitol Min-Foam 1X®, and Cassia oil.

Another embodiment comprises cyclosporin A, Tergitol Min-Foam 2X®, and Cassia oil.

Another embodiment comprises cyclosporin A, Triton CF-21®, and Cassia oil.

Another embodiment comprises cyclosporin A, Triton CF-76®, and Cassia oil.

Another embodiment comprises cyclosporin A, Triton XL-80N®, and Cassia oil.

Another embodiment comprises cyclosporin A, Witconol NS-98®, and Cassia oil.

Another embodiment comprises cyclosporin A, Witconol NS-108LQ®, and Cassia oil.

Another embodiment comprises cyclosporin A, Witconol NS-145®, and Cassia oil.

Another embodiment comprises cyclosporin A, Witconol NS-179®, and Cassia oil.

Another embodiment comprises cyclosporin A, a Protein-based Surfactant, and Cassia oil.

Another embodiment comprises cyclosporin A, AminoFoam W®, and Cassia oil.

Another embodiment comprises cyclosporin A, Amiter LGOD-2®, and Cassia oil.

Another embodiment comprises cyclosporin A, Amiter LGS-2®, and Cassia oil.

Another embodiment comprises cyclosporin A, Amiter LGS-5®, and Cassia oil.

Another embodiment comprises cyclosporin A, Lamepon S®, and Cassia oil.

Another embodiment comprises cyclosporin A, Lamepon S®, and Cassia oil.

Another embodiment comprises cyclosporin A, Maypon 4C®, and Cassia oil.

Another embodiment comprises cyclosporin A, May-Tein C®, and Cassia oil.

Another embodiment comprises cyclosporin A, May-Tein CT®, and Cassia oil.

Another embodiment comprises cyclosporin A, May-Tein KTS®, and Cassia oil.

Another embodiment comprises cyclosporin A, May-Tein SY®, and Cassia oil.

Another embodiment comprises cyclosporin A, Plantapon S®, and Cassia oil.

Another embodiment comprises cyclosporin A, Proteol APL®, and Cassia oil.

Another embodiment comprises cyclosporin A, Proteol OAT®, and Cassia oil.

Another embodiment comprises cyclosporin A, Pyroter CPI-40®, and Cassia oil.

Another embodiment comprises cyclosporin A, Pyroter GPI-25®, and Cassia oil.

Another embodiment comprises cyclosporin A, Supro-Tein S®, and Cassia oil.

Another embodiment comprises cyclosporin A, Supro-Tein V®, and Cassia oil.

Another embodiment comprises cyclosporin A, ® and Cassia oil.

Another embodiment comprises cyclosporin A, a Sarcosine Derivative®, and Cassia oil.

Another embodiment comprises cyclosporin A, Crodasinic LS-30®, and Cassia oil.

Another embodiment comprises cyclosporin A, Vanseal CS®, and Cassia oil.

Another embodiment comprises cyclosporin A, Vanseal LS®, and Cassia oil.

Another embodiment comprises cyclosporin A, Vanseal MS®, and Cassia oil.

Another embodiment comprises cyclosporin A, Vanseal NACS-30®, and Cassia oil.

Another embodiment comprises cyclosporin A, Vanseal NALS-95®, and Cassia oil.

Another embodiment comprises cyclosporin A, Vanseal OS®, and Cassia oil.

Another embodiment comprises cyclosporin A, ® and Cassia oil.

Another embodiment comprises cyclosporin A, a Silicone-based Surfactant®, and Cassia oil.

Another embodiment comprises cyclosporin A, Abil-B-9950®, and Cassia oil.

Another embodiment comprises cyclosporin A, Abil Care 85®, and Cassia oil.

Another embodiment comprises cyclosporin A, Abil EM 90®, and Cassia oil.

Another embodiment comprises cyclosporin A, Abil EM 97®, and Cassia oil.

Another embodiment comprises cyclosporin A, Abil WE-09®, and Cassia oil.

Another embodiment comprises cyclosporin A, Dow Corning 1248 Fluid®, and Cassia oil.

Another embodiment comprises cyclosporin A, Dow Corning 3225C®, and Cassia oil.

Another embodiment comprises cyclosporin A, Dow Corning 5200®, and Cassia oil.

Another embodiment comprises cyclosporin A, Dow Corning Q4-3667®, and Cassia oil.

Another embodiment comprises cyclosporin A, Monasil PCA®, and Cassia oil.

Another embodiment comprises cyclosporin A, Monasil PDM®, and Cassia oil.

Another embodiment comprises cyclosporin A, Monasil PLN®, and Cassia oil.

Another embodiment comprises cyclosporin A, Polyderm PPI-SI-WS®, and Cassia oil.

Another embodiment comprises cyclosporin A, Troysol 380W®, and Cassia oil.

Another embodiment comprises cyclosporin A, Troysol S366®, and Cassia oil.

Another embodiment comprises cyclosporin A, a Sorbitan Derivative, and Cassia oil.

Another embodiment comprises cyclosporin A, Alkamuls SML®, and Cassia oil.

Another embodiment comprises cyclosporin A, Alkamuls SMO®, and Cassia oil.

Another embodiment comprises cyclosporin A, Alkamuls STO®, and Cassia oil.

Another embodiment comprises cyclosporin A, Arlacel 20®, and Cassia oil.

Another embodiment comprises cyclosporin A, Arlacel 40®, and Cassia oil.

Another embodiment comprises cyclosporin A, Arlacel 60®, and Cassia oil.

Another embodiment comprises cyclosporin A, Arlacel 80®, and Cassia oil.

Another embodiment comprises cyclosporin A, Arlacel C®, and Cassia oil.

Another embodiment comprises cyclosporin A, Armul 21®, and Cassia oil.

Another embodiment comprises cyclosporin A, Atlox 80®, and Cassia oil.

Another embodiment comprises cyclosporin A, Atlox 847®, and Cassia oil.

Another embodiment comprises cyclosporin A, Atlox 1045A®, and Cassia oil.

Another embodiment comprises cyclosporin A, Canarcel 20®, and Cassia oil.

Another embodiment comprises cyclosporin A, Canarcel 60®, and Cassia oil.

Another embodiment comprises cyclosporin A, Canarcel 80®, and Cassia oil.

Another embodiment comprises cyclosporin A, Canarcel TW 20®, and Cassia oil.

Another embodiment comprises cyclosporin A, Canarcel TW 60®, and Cassia oil.

Another embodiment comprises cyclosporin A, Canarcel TW 80®, and Cassia oil.

Another embodiment comprises cyclosporin A, Coladet BSB-P®, and Cassia oil.

Another embodiment comprises cyclosporin A, Customulse O-20®, and Cassia oil.

Another embodiment comprises cyclosporin A, Dehymuls E®, and Cassia oil.

Another embodiment comprises cyclosporin A, DeSotan SMO®, and Cassia oil.

Another embodiment comprises cyclosporin A, DeSotan SMO-20®, and Cassia oil.

Another embodiment comprises cyclosporin A, DeSotan SMT®, and Cassia oil.

Another embodiment comprises cyclosporin A, DeSotan SMT-20®, and Cassia oil.

Another embodiment comprises cyclosporin A, Durfax 60®, and Cassia oil.

Another embodiment comprises cyclosporin A, Duraax 65®, and Cassia oil.

Another embodiment comprises cyclosporin A, Durfax 80®, and Cassia oil.

Another embodiment comprises cyclosporin A, Durtan 60®, and Cassia oil.

Another embodiment comprises cyclosporin A, Durtan 65®, and Cassia oil.

Another embodiment comprises cyclosporin A, Liposorb L®, and Cassia oil.

Another embodiment comprises cyclosporin A, Liposorb L-10®, and Cassia oil.

Another embodiment comprises cyclosporin A, Liposorb L-20®, and Cassia oil.

Another embodiment comprises cyclosporin A, Liposorb O®, and Cassia oil.

Another embodiment comprises cyclosporin A, Liposorb O-20®, and Cassia oil.

Another embodiment comprises cyclosporin A, Liposorb F®, and Cassia oil.

Another embodiment comprises cyclosporin A, Liposorb P-20®, and Cassia oil.

Another embodiment comprises cyclosporin A, Liposorb S®, and Cassia oil.

Another embodiment comprises cyclosporin A, Liposorb S-20®, and Cassia oil.

Another embodiment comprises cyclosporin A, Liposorb SQO®, and Cassia oil.

Another embodiment comprises cyclosporin A, Liposorb TO®, and Cassia oil.

Another embodiment comprises cyclosporin A, Liposorb TS®, and Cassia oil.

Another embodiment comprises cyclosporin A, Liposorb TS-20®, and Cassia oil.

Another embodiment comprises cyclosporin A, Lumisorb PS®, and Cassia oil.

Another embodiment comprises cyclosporin A, Lumisorb SMO (T)®, and Cassia oil.

Another embodiment comprises cyclosporin A, Lumisorb SMS K®, and Cassia oil.

Another embodiment comprises cyclosporin A, Lumisorb SSO®, and Cassia oil.

Another embodiment comprises cyclosporin A, Lumisorb STS K®, and Cassia oil.

Another embodiment comprises cyclosporin A, Lumisorb STT®, and Cassia oil.

Another embodiment comprises cyclosporin A, Magrabar SMO®, and Cassia oil.

Another embodiment comprises cyclosporin A, Magrabar SMO-VEG®, and Cassia oil.

Another embodiment comprises cyclosporin A, Magrabar SMT®, and Cassia oil.

Another embodiment comprises cyclosporin A, Magrabar STO®, and Cassia oil.

Another embodiment comprises cyclosporin A, Miracare BC-27®, and Cassia oil.

Another embodiment comprises cyclosporin A, Ritabate 20®, and Cassia oil.

Another embodiment comprises cyclosporin A, Ritabate 40®, and Cassia oil.

Another embodiment comprises cyclosporin A, Ritabate 60®, and Cassia oil.

Another embodiment comprises cyclosporin A, Ritabate 80®, and Cassia oil.

Another embodiment comprises cyclosporin A, T-Maz®, and Cassia oil.

Another embodiment comprises cyclosporin A, Tego SML®, and Cassia oil.

Another embodiment comprises cyclosporin A, Tego SML 20®, and Cassia oil.

Another embodiment comprises cyclosporin A, Tego SMO 80 V®, and Cassia oil.

Another embodiment comprises cyclosporin A, Tego SMO V®, and Cassia oil.

Another embodiment comprises cyclosporin A, Tego SMS®, and Cassia oil.

Another embodiment comprises cyclosporin A, Tego STO V®, and Cassia oil.

Another embodiment comprises cyclosporin A, Tween 21®, and Cassia oil.

Another embodiment comprises cyclosporin A, Tween 40®, and Cassia oil.

Another embodiment comprises cyclosporin A, Tween 60®, and Cassia oil.

Another embodiment comprises cyclosporin A, Tween 60 K®, and Cassia oil.

Another embodiment comprises cyclosporin A, Tween 61®, and Cassia oil.

Another embodiment comprises cyclosporin A, Tween 65®, and Cassia oil.

Another embodiment comprises cyclosporin A, Tween 80®, and Cassia oil.

Another embodiment comprises cyclosporin A, Tween 80 K®, and Cassia oil.

Another embodiment comprises cyclosporin A, Tween 81®, and Cassia oil.

Another embodiment comprises cyclosporin A, Tween 85®, and Cassia oil.

Another embodiment comprises cyclosporin A, a Sucrose or Glucose Ester, or Derivative thereof, and Cassia oil.

Another embodiment comprises cyclosporin A, DeSulf GOS-P-60WCG®, and Cassia oil.

Another embodiment comprises cyclosporin A, Glucam E-20 Distearate®, and Cassia oil.

Another embodiment comprises cyclosporin A, Glucamate DOE-120®, and Cassia oil.

Another embodiment comprises cyclosporin A, Glucamate SSE-20®, and Cassia oil.

Another embodiment comprises cyclosporin A, Glucate DO®, and Cassia oil.

Another embodiment comprises cyclosporin A, Glucate SS®, and Cassia oil.

Another embodiment comprises cyclosporin A, Glucopon 425 UP®, and Cassia oil.

Another embodiment comprises cyclosporin A, Isolan IS®, and Cassia oil.

Another embodiment comprises cyclosporin A, Mazon 40®, and Cassia oil.

Another embodiment comprises cyclosporin A, Montanov 82®, and Cassia oil.

Another embodiment comprises cyclosporin A, Montanov 202®, and Cassia oil.

Another embodiment comprises cyclosporin A, Montanov S®, and Cassia oil.

Another embodiment comprises cyclosporin A, Rheozan®, and Cassia oil.

Another embodiment comprises cyclosporin A, Simulsol AS 48®, and Cassia oil.

Another embodiment comprises cyclosporin A, Simulsol SL 4®, and Cassia oil.

Another embodiment comprises cyclosporin A, Simulsol SL 10®, and Cassia oil.

Another embodiment comprises cyclosporin A, Simulsol SL 11 W®, and Cassia oil.

Another embodiment comprises cyclosporin A, Simulsol SL 55®, and Cassia oil.

Another embodiment comprises cyclosporin A, Suga Nate 100 and 160®, and Cassia oil.

Another embodiment comprises cyclosporin A, Tego Care 450®, and Cassia oil.

Another embodiment comprises cyclosporin A, Tego Care CG 90®, and Cassia oil.

Another embodiment comprises cyclosporin A, Tego Care PS®, and Cassia oil.

Another embodiment comprises cyclosporin A, Tegosoft PSE 141 G®, and Cassia oil.

Another embodiment comprises cyclosporin A, Tegotens G 826®, and Cassia oil.

Another embodiment comprises cyclosporin A, Triton BG-10 (70%)®, and Cassia oil.

Another embodiment comprises cyclosporin A, Triton CG-110 (60%)®, and Cassia oil.

Another embodiment comprises cyclosporin A, Wickenol 545®, and Cassia oil.

Another embodiment comprises cyclosporin A, an alcohol, and Cinnamon oil.

Another embodiment comprises cyclosporin A, Diglycerol®, and Cinnamon oil.

Another embodiment comprises cyclosporin A, Hetoxide GT-80®, and Cinnamon oil.

Another embodiment comprises cyclosporin A, Lexemul BEO®, and Cinnamon oil.

Another embodiment comprises cyclosporin A, Polyglycerol-3®, and Cinnamon oil.

Another embodiment comprises cyclosporin A, Redicote E Series®, and Cinnamon oil.

Another embodiment comprises cyclosporin A, Simulsol OX 1005L®, and Cinnamon oil.

Another embodiment comprises cyclosporin A, Stanfax 567®, and Cinnamon oil.

Another embodiment comprises cyclosporin A, TA-1618®, and Cinnamon oil.

Another embodiment comprises cyclosporin A, Witconol H-31A®, and Cinnamon oil.

Another embodiment comprises cyclosporin A, an amine oxide, and Cinnamon oil.

Another embodiment comprises cyclosporin A, AO-405®, and Cinnamon oil.

Another embodiment comprises cyclosporin A, AO-455®, and Cinnamon oil.

Another embodiment comprises cyclosporin A, AO 728 Special®, and Cinnamon oil.

Another embodiment comprises cyclosporin A, Barlox 12®, and Cinnamon oil.

Another embodiment comprises cyclosporin A, Barlox 14®, and Cinnamon oil.

Another embodiment comprises cyclosporin A, Burcoxide Lo®, and Cinnamon oil.

Another embodiment comprises cyclosporin A, Caloxamine LO®, and Cinnamon oil.

Another embodiment comprises cyclosporin A, Chemoxide CAW®, and Cinnamon oil.

Another embodiment comprises cyclosporin A, Chemoxide LM-30®, and Cinnamon oil.

Another embodiment comprises cyclosporin A, Chemoxide LO®, and Cinnamon oil.

Another embodiment comprises cyclosporin A, Chemoxide MO®, and Cinnamon oil.

Another embodiment comprises cyclosporin A, Colalux CAO-35®, and Cinnamon oil.

Another embodiment comprises cyclosporin A, Colalux LO®, and Cinnamon oil.

Another embodiment comprises cyclosporin A, DeMox CAPO®, and Cinnamon oil.

Another embodiment comprises cyclosporin A, DeMox CSG-30®, and Cinnamon oil.

Another embodiment comprises cyclosporin A, DeMox LAO®, and Cinnamon oil.

Another embodiment comprises cyclosporin A, Emcol L®, and Cinnamon oil.

Another embodiment comprises cyclosporin A, Empigen OB®, and Cinnamon oil.

Another embodiment comprises cyclosporin A, Empigen OS/A®, and Cinnamon oil.

Another embodiment comprises cyclosporin A, Foamox CDO®, and Cinnamon oil.

Another embodiment comprises cyclosporin A, Foamox DMM®, and Cinnamon oil.

Another embodiment comprises cyclosporin A, Foamox DMS®, and Cinnamon oil.

Another embodiment comprises cyclosporin A, Genaminox KC®, and Cinnamon oil.

Another embodiment comprises cyclosporin A, Genaminox LA®, and Cinnamon oil.

Another embodiment comprises cyclosporin A, Hartofoam SAO®, and Cinnamon oil.

Another embodiment comprises cyclosporin A, Hartox DMCD®, and Cinnamon oil.

Another embodiment comprises cyclosporin A, Lipowax DAT®, and Cinnamon oil.

Another embodiment comprises cyclosporin A, Lipowax PB Pastilles®, and Cinnamon oil.

Another embodiment comprises cyclosporin A, Mackamine C8®, and Cinnamon oil.

Another embodiment comprises cyclosporin A, Mackamine C10®, and Cinnamon oil.

Another embodiment comprises cyclosporin A, Mackamine C14®, and Cinnamon oil.

Another embodiment comprises cyclosporin A, Mackamine CAO®, and Cinnamon oil.

Another embodiment comprises cyclosporin A, Mackamine CO®, and Cinnamon oil.

Another embodiment comprises cyclosporin A, Mackamine LO®, and Cinnamon oil.

Another embodiment comprises cyclosporin A, Mackamine O2®, and Cinnamon oil.

Another embodiment comprises cyclosporin A, Mackamine SAO®, and Cinnamon oil.

Another embodiment comprises cyclosporin A, Mackamine SO®, and Cinnamon oil.

Another embodiment comprises cyclosporin A, Mazox KCAO®, and Cinnamon oil.

Another embodiment comprises cyclosporin A, Monalac MO®, and Cinnamon oil.

Another embodiment comprises cyclosporin A, Norfox LDA®, and Cinnamon oil.

Another embodiment comprises cyclosporin A, Rhodamox LO®, and Cinnamon oil.

Another embodiment comprises cyclosporin A, Schercamox C-AA®, and Cinnamon oil.

Another embodiment comprises cyclosporin A, Schercamox DMA®, and Cinnamon oil.

Another embodiment comprises cyclosporin A, Schercamox DML®, and Cinnamon oil.

Another embodiment comprises cyclosporin A, Schercamox DMM®, and Cinnamon oil.

Another embodiment comprises cyclosporin A, Schercamox DMS®, and Cinnamon oil.

Another embodiment comprises cyclosporin A, Tegotens DO®, and Cinnamon oil.

Another embodiment comprises cyclosporin A, Tomah AO-14-2®, and Cinnamon oil.

Another embodiment comprises cyclosporin A, Triaminox CDO®, and Cinnamon oil.

Another embodiment comprises cyclosporin A, a block polymer, and Cinnamon oil.

Another embodiment comprises cyclosporin A, AL 2070®, and Cinnamon oil.

Another embodiment comprises cyclosporin A, Antarox 17-R-2®, and Cinnamon oil.

Another embodiment comprises cyclosporin A, Antarox 25-R-2®, and Cinnamon oil.

Another embodiment comprises cyclosporin A, Antarox 31-R-1®, and Cinnamon oil.

Another embodiment comprises cyclosporin A, Antarox P-84®, and Cinnamon oil.

Another embodiment comprises cyclosporin A, Antarox P-104/H®, and Cinnamon oil.

Another embodiment comprises cyclosporin A, Arnox BP-Series®, and Cinnamon oil.

Another embodiment comprises cyclosporin A, Chemonic 435®, and Cinnamon oil.

Another embodiment comprises cyclosporin A, Chemonic D-25®, and Cinnamon oil.

Another embodiment comprises cyclosporin A, Chemonic PL Series®, and Cinnamon oil.

Another embodiment comprises cyclosporin A, Ethox L-121®, and Cinnamon oil.

Another embodiment comprises cyclosporin A, Ethox L-122®, and Cinnamon oil.

Another embodiment comprises cyclosporin A, Genapol PF-10®, and Cinnamon oil.

Another embodiment comprises cyclosporin A, Genapol PF-20®, and Cinnamon oil.

Another embodiment comprises cyclosporin A, Genapol PF-40A®, and Cinnamon oil.

Another embodiment comprises cyclosporin A, Norfox 2-LF®, and Cinnamon oil.

Another embodiment comprises cyclosporin A, Simulsol NW 342®, and Cinnamon oil.

Another embodiment comprises cyclosporin A, T-Det BP-1®, and Cinnamon oil.

Another embodiment comprises cyclosporin A, T-Det XD®, and Cinnamon oil.

Another embodiment comprises cyclosporin A, T-Det XH®, and Cinnamon oil.

Another embodiment comprises cyclosporin A, Triton CF-32®, and Cinnamon oil.

Another embodiment comprises cyclosporin A, Witconol 171®, and Cinnamon oil.

Another embodiment comprises cyclosporin A, Witconol 324®, and Cinnamon oil.

Another embodiment comprises cyclosporin A, Witconol 324D®, and Cinnamon oil.

Another embodiment comprises cyclosporin A, Witconol PD-2000®, and Cinnamon oil.

Another embodiment comprises cyclosporin A, a Carboxylated Alcohol or Alkylphenol Ethoxylate, and Cinnamon oil.

Another embodiment comprises cyclosporin A, Emcol CN-6®, and Cinnamon oil.

Another embodiment comprises cyclosporin A, Ethcarb®, and Cinnamon oil.

Another embodiment comprises cyclosporin A, Gemtex WNT-Conc®, and Cinnamon oil.

Another embodiment comprises cyclosporin A, Incrodet TD7-C®, and Cinnamon oil.

Another embodiment comprises cyclosporin A, Marlinat CM 105/80®, and Cinnamon oil.

Another embodiment comprises cyclosporin A, Marlowet 1072®, and Cinnamon oil.

Another embodiment comprises cyclosporin A, Marlowet 4530®, and Cinnamon oil.

Another embodiment comprises cyclosporin A, Marlowet 4530 LF®, and Cinnamon oil.

Another embodiment comprises cyclosporin A, Marlowet 4534®, and Cinnamon oil.

Another embodiment comprises cyclosporin A, Marlowet 4538®, and Cinnamon oil.

Another embodiment comprises cyclosporin A, Marlowet 4539®, and Cinnamon oil.

Another embodiment comprises cyclosporin A, Marlowet 4539 LF®, and Cinnamon oil.

Another embodiment comprises cyclosporin A, Marlowet 4541®, and Cinnamon oil.

Another embodiment comprises cyclosporin A, Miranate LEC-80®, and Cinnamon oil.

Another embodiment comprises cyclosporin A, Sandopan B®, and Cinnamon oil.

Another embodiment comprises cyclosporin A, Sandopan B Modified®, and Cinnamon oil.

Another embodiment comprises cyclosporin A, Sandopan LS-24 Gel®, and Cinnamon oil.

Another embodiment comprises cyclosporin A, Surfine T-A®, and Cinnamon oil.

Another embodiment comprises cyclosporin A, a Carboxylic Acid/Fatty Acid, and Cinnamon oil.

Another embodiment comprises cyclosporin A, Colaterge RAM®, and Cinnamon oil.

Another embodiment comprises cyclosporin A, Colatrope INC®, and Cinnamon oil.

Another embodiment comprises cyclosporin A, Crodacid B®, and Cinnamon oil.

Another embodiment comprises cyclosporin A, DeTrope CA-100®, and Cinnamon oil.

Another embodiment comprises cyclosporin A, Latol MTO®, and Cinnamon oil.

Another embodiment comprises cyclosporin A, Lumulse CC-33 K®, and Cinnamon oil.

Another embodiment comprises cyclosporin A, Mulls 2218®, and Cinnamon oil.

Another embodiment comprises cyclosporin A, OL-600®, and Cinnamon oil.

Another embodiment comprises cyclosporin A, OL-800®, and Cinnamon oil.

Another embodiment comprises cyclosporin A, R-910®, and Cinnamon oil.

Another embodiment comprises cyclosporin A, S-210®, and Cinnamon oil.

Another embodiment comprises cyclosporin A, Sandopan DTC Acid®, and Cinnamon oil.

Another embodiment comprises cyclosporin A, Sandopan LS 24 N®, and Cinnamon oil.

Another embodiment comprises cyclosporin A, Sandopan MA-18®, and Cinnamon oil.

Another embodiment comprises cyclosporin A, an Ethoxylated Alcohol, and Cinnamon oil.

Another embodiment comprises cyclosporin A, Adsee 799®, and Cinnamon oil.

Another embodiment comprises cyclosporin A, Adsee 799®, and Cinnamon oil.

Another embodiment comprises cyclosporin A, Alfonic 610-3.5®, and Cinnamon oil.

Another embodiment comprises cyclosporin A, Alfonic 810-2®, and Cinnamon oil.

Another embodiment comprises cyclosporin A, Alfonic 810-6®, and Cinnamon oil.

Another embodiment comprises cyclosporin A, Alfonic 1012-3®, and Cinnamon oil.

Another embodiment comprises cyclosporin A, Alfonic 1012-5®, and Cinnamon oil.

Another embodiment comprises cyclosporin A, Alfonic 1216CO-1.5®, and Cinnamon oil.

Another embodiment comprises cyclosporin A, Alfonic 1216CO-7®, and Cinnamon oil.

Another embodiment comprises cyclosporin A, Alfonic 1412-3®, and Cinnamon oil.

Another embodiment comprises cyclosporin A, Alfonic 1412-7®, and Cinnamon oil.

Another embodiment comprises cyclosporin A, Arlasolve 200®, and Cinnamon oil.

Another embodiment comprises cyclosporin A, Arlasolve 200 Liquid®, and Cinnamon oil.

Another embodiment comprises cyclosporin A, Armix 180-C®, and Cinnamon oil.

Another embodiment comprises cyclosporin A, Armix 183®, and Cinnamon oil.

Another embodiment comprises cyclosporin A, Armul 2404®, and Cinnamon oil.

Another embodiment comprises cyclosporin A, Armul 2404®, and Cinnamon oil.

Another embodiment comprises cyclosporin A, Atlas EMJ-C®, and Cinnamon oil.

Another embodiment comprises cyclosporin A, Atlas G-2109®, and Cinnamon oil.

Another embodiment comprises cyclosporin A, Atlas G-3886®, and Cinnamon oil.

Another embodiment comprises cyclosporin A, Atlas G-3890®, and Cinnamon oil.

Another embodiment comprises cyclosporin A, Bio Soft E-200®, and Cinnamon oil.

Another embodiment comprises cyclosporin A, Bio Soft E-300®, and Cinnamon oil.

Another embodiment comprises cyclosporin A, Bio Soft E-400®, and Cinnamon oil.

Another embodiment comprises cyclosporin A, Bio Soft EN 600®, and Cinnamon oil.

Another embodiment comprises cyclosporin A, Bio Soft TD-400®, and Cinnamon oil.

Another embodiment comprises cyclosporin A, Bio Soft TD-630®, and Cinnamon oil.

Another embodiment comprises cyclosporin A, Brij 30®, and Cinnamon oil.

Another embodiment comprises cyclosporin A, Brij 52®, and Cinnamon oil.

Another embodiment comprises cyclosporin A, Brij 56®, and Cinnamon oil.

Another embodiment comprises cyclosporin A, Brij 58®, and Cinnamon oil.

Another embodiment comprises cyclosporin A, Brij 72®, and Cinnamon oil.

Another embodiment comprises cyclosporin A, Brij 76®, and Cinnamon oil.

Another embodiment comprises cyclosporin A, Brij 78®, and Cinnamon oil.

Another embodiment comprises cyclosporin A, Brij 93®, and Cinnamon oil.

Another embodiment comprises cyclosporin A, Brij 97®, and Cinnamon oil.

Another embodiment comprises cyclosporin A, Brij 98®, and Cinnamon oil.

Another embodiment comprises cyclosporin A, Brij 700®, and Cinnamon oil.

Another embodiment comprises cyclosporin A, Brij 700 S®, and Cinnamon oil.

Another embodiment comprises cyclosporin A, Brij 721®, and Cinnamon oil.

Another embodiment comprises cyclosporin A, Brij 721 S®, and Cinnamon oil.

Another embodiment comprises cyclosporin A, Burcoterge CDG®, and Cinnamon oil.

Another embodiment comprises cyclosporin A, Canasol AT 600®, and Cinnamon oil.

Another embodiment comprises cyclosporin A, Canasol AT 800®, and Cinnamon oil.

Another embodiment comprises cyclosporin A, Canasol AT 1200®, and Cinnamon oil.

Another embodiment comprises cyclosporin A, Canasol BJ 35®, and Cinnamon oil.

Another embodiment comprises cyclosporin A, Canasol BJ 36®, and Cinnamon oil.

Another embodiment comprises cyclosporin A, Canasol BJ 52®, and Cinnamon oil.

Another embodiment comprises cyclosporin A, Canasol BJ 58®, and Cinnamon oil.

Another embodiment comprises cyclosporin A, Canasbl BJ 72®, and Cinnamon oil.

Another embodiment comprises cyclosporin A, Canasol BJ 78®, and Cinnamon oil.

Another embodiment comprises cyclosporin A, Canasol BJ 98®, and Cinnamon oil.

Another embodiment comprises cyclosporin A, Canasol BJ 307®, and Cinnamon oil.

Another embodiment comprises cyclosporin A, Cerfak 1400®, and Cinnamon oil.

Another embodiment comprises cyclosporin A, Cetomacrogol 1000 BP®, and Cinnamon oil.

Another embodiment comprises cyclosporin A, Chemonic C-2®, and Cinnamon oil.

Another embodiment comprises cyclosporin A, Chemonic C-10®, and Cinnamon oil.

Another embodiment comprises cyclosporin A, Chemonic C-20®, and Cinnamon oil.

Another embodiment comprises cyclosporin A, Chemonic CT-12®, and Cinnamon oil.

Another embodiment comprises cyclosporin A, Chemonic CT-20®, and Cinnamon oil.

Another embodiment comprises cyclosporin A, Chemonic CT-30®, and Cinnamon oil.

Another embodiment comprises cyclosporin A, Chemonic CT-55®, and Cinnamon oil.

Another embodiment comprises cyclosporin A, Chemonic G-7®, and Cinnamon oil.

Another embodiment comprises cyclosporin A, Chemonic G-26®, and Cinnamon oil.

Another embodiment comprises cyclosporin A, Chemonic L-4®, and Cinnamon oil.

Another embodiment comprises cyclosporin A, Chemonic L-7®, and Cinnamon oil.

Another embodiment comprises cyclosporin A, Chemonic L-12®, and Cinnamon oil.

Another embodiment comprises cyclosporin A, Chemonic L-23®, and Cinnamon oil.

Another embodiment comprises cyclosporin A, Chemonic O-2®, and Cinnamon oil.

Another embodiment comprises cyclosporin A, Chemonic O-5®, and Cinnamon oil.

Another embodiment comprises cyclosporin A, Chemonic O-10®, and Cinnamon oil.

Another embodiment comprises cyclosporin A, Chemonic O-20®, and Cinnamon oil.

Another embodiment comprises cyclosporin A, Chemonic S-2®, and Cinnamon oil.

Another embodiment comprises cyclosporin A, Chemonic S-10®, and Cinnamon oil.

Another embodiment comprises cyclosporin A, Chemonic S-20®, and Cinnamon oil.

Another embodiment comprises cyclosporin A, Colamulse FE®, and Cinnamon oil.

Another embodiment comprises cyclosporin A, Cremophor A 20®, and Cinnamon oil.

Another embodiment comprises cyclosporin A, Cremophor SA 2®, and Cinnamon oil.

Another embodiment comprises cyclosporin A, Dehydol 100®, and Cinnamon oil.

Another embodiment comprises cyclosporin A, Dehydol O-4®, and Cinnamon oil.

Another embodiment comprises cyclosporin A, Delonic C-18®, and Cinnamon oil.

Another embodiment comprises cyclosporin A, DeSonic 6T®, and Cinnamon oil.

Another embodiment comprises cyclosporin A, DeSonic 9D®, and Cinnamon oil.

Another embodiment comprises cyclosporin A, DeSonic 9T®, and Cinnamon oil.

Another embodiment comprises cyclosporin A, DeSonic 12D®, and Cinnamon oil.

Another embodiment comprises cyclosporin A, DeSonic 12T®, and Cinnamon oil.

Another embodiment comprises cyclosporin A, DeSonic 15T®, and Cinnamon oil.

Another embodiment comprises cyclosporin A, DeSonic TDA-9®, and Cinnamon oil.

Another embodiment comprises cyclosporin A, DeThox GLG-7®, and Cinnamon oil.

Another embodiment comprises cyclosporin A, DeThox GLG-26®, and Cinnamon oil.

Another embodiment comprises cyclosporin A, DeThox LA-4®, and Cinnamon oil.

Another embodiment comprises cyclosporin A, DeThox LA-23®, and Cinnamon oil.

Another embodiment comprises cyclosporin A, DeThox SA-80®, and Cinnamon oil.

Another embodiment comprises cyclosporin A, Disponil O5®, and Cinnamon oil.

Another embodiment comprises cyclosporin A, Eccoterge EO-41B®, and Cinnamon oil.

Another embodiment comprises cyclosporin A, Empilan KA2.5/90®, and Cinnamon oil.

Another embodiment comprises cyclosporin A, Empilan KA5/90®, and Cinnamon oil.

Another embodiment comprises cyclosporin A, Empilan KM-20®, and Cinnamon oil.

Another embodiment comprises cyclosporin A, Empilan KM-50®, and Cinnamon oil.

Another embodiment comprises cyclosporin A, Empilan L-23®, and Cinnamon oil.

Another embodiment comprises cyclosporin A, Ethylan 25-3®, and Cinnamon oil.

Another embodiment comprises cyclosporin A, Ethylan 1204®, and Cinnamon oil.

Another embodiment comprises cyclosporin A, Ethylan DA-4®, and Cinnamon oil.

Another embodiment comprises cyclosporin A, Ethylan LA-230®, and Cinnamon oil.

Another embodiment comprises cyclosporin A, Ethylan SN®, and Cinnamon oil.

Another embodiment comprises cyclosporin A, Ethylan TD-60®, and Cinnamon oil.

Another embodiment comprises cyclosporin A, Ethylan TD-100®, and Cinnamon oil.

Another embodiment comprises cyclosporin A, Ethylan TD-1407®, and Cinnamon oil.

Another embodiment comprises cyclosporin A, Eumulgin B1®, and Cinnamon oil.

Another embodiment comprises cyclosporin A, Eumulgin B2®, and Cinnamon oil.

Another embodiment comprises cyclosporin A, Eumulgin B3®, and Cinnamon oil.

Another embodiment comprises cyclosporin A, Eumulgin O-10®, and Cinnamon oil.

Another embodiment comprises cyclosporin A, Flo Mo 80/20®, and Cinnamon oil.

Another embodiment comprises cyclosporin A, Flo Mo Low Foam®, and Cinnamon oil.

Another embodiment comprises cyclosporin A, Forlan C-24®, and Cinnamon oil.

Another embodiment comprises cyclosporin A, Genapol 1454®, and Cinnamon oil.

Another embodiment comprises cyclosporin A, Genapol BA-020®, and Cinnamon oil.

Another embodiment comprises cyclosporin A, Genapol BA-040®, and Cinnamon oil.

Another embodiment comprises cyclosporin A, Genapol C-100®, and Cinnamon oil.

Another embodiment comprises cyclosporin A, Genapol DA 060®, and Cinnamon oil.

Another embodiment comprises cyclosporin A, Genapol HS 020®, and Cinnamon oil.

Another embodiment comprises cyclosporin A, Genapol HS 200®, and Cinnamon oil.

Another embodiment comprises cyclosporin A, Genapol ID-040®, and Cinnamon oil.

Another embodiment comprises cyclosporin A, Genapol ID-060®, and Cinnamon oil.

Another embodiment comprises cyclosporin A, Genapol ID-090®, and Cinnamon oil.

Another embodiment comprises cyclosporin A, Genapol LA 010®, and Cinnamon oil.

Another embodiment comprises cyclosporin A, Genapol LA 020®, and Cinnamon oil.

Another embodiment comprises cyclosporin A, Genapol LA 030®, and Cinnamon oil.

Another embodiment comprises cyclosporin A, Genapol LA 040®, and Cinnamon oil.

Another embodiment comprises cyclosporin A, Genapol LA 050®, and Cinnamon oil.

Another embodiment comprises cyclosporin A, Genapol LA 060®, and Cinnamon oil.

Another embodiment comprises cyclosporin A, Genapol LA 070®, and Cinnamon oil.

Another embodiment comprises cyclosporin A, Genapol LA 070S8, and Cinnamon oil.

Another embodiment comprises cyclosporin A, Genapol LA 230®, and Cinnamon oil.

Another embodiment comprises cyclosporin A, Genapol O020®, and Cinnamon oil.

Another embodiment comprises cyclosporin A, Genapol O050®, and Cinnamon oil.

Another embodiment comprises cyclosporin A, Genapol O100®, and Cinnamon oil.

Another embodiment comprises cyclosporin A, Genapol O200®, and Cinnamon oil.

Another embodiment comprises cyclosporin A, Genapol SA 030®, and Cinnamon oil.

Another embodiment comprises cyclosporin A, Genapol SA 120®, and Cinnamon oil.

Another embodiment comprises cyclosporin A, Genapol T-020®, and Cinnamon oil.

Another embodiment comprises cyclosporin A, Genapol UD-030®, and Cinnamon oil.

Another embodiment comprises cyclosporin A, Genapol UD-050®, and Cinnamon oil.

Another embodiment comprises cyclosporin A, Genapol UD-070®, and Cinnamon oil.

Another embodiment comprises cyclosporin A, Genapol UD-079®, and Cinnamon oil.

Another embodiment comprises cyclosporin A, Genapol UD-080®, and Cinnamon oil.

Another embodiment comprises cyclosporin A, Genapol UD-110®, and Cinnamon oil.

Another embodiment comprises cyclosporin A, Genapol X 030®, and Cinnamon oil.

Another embodiment comprises cyclosporin A, Genapol X 050®, and Cinnamon oil.

Another embodiment comprises cyclosporin A, Genapol X 060®, and Cinnamon oil.

Another embodiment comprises cyclosporin A, Genapol X 070®, and Cinnamon oil.

Another embodiment comprises cyclosporin A, Genapol X 080®, and Cinnamon oil.

Another embodiment comprises cyclosporin A, Genapol X 100®, and Cinnamon oil.

Another embodiment comprises cyclosporin A, Genapol X159®, and Cinnamon oil.

Another embodiment comprises cyclosporin A, Generol 122 E5®, and Cinnamon oil.

Another embodiment comprises cyclosporin A, Generol 122 E25®, and Cinnamon oil.

Another embodiment comprises cyclosporin A, Hostacerin T-3®, and Cinnamon oil.

Another embodiment comprises cyclosporin A, Iconol LF 110®, and Cinnamon oil.

Another embodiment comprises cyclosporin A, Incropol CS-20®, and Cinnamon oil.

Another embodiment comprises cyclosporin A, Lexemul CS-20®, and Cinnamon oil.

Another embodiment comprises cyclosporin A, Liponic EG-1®, and Cinnamon oil.

Another embodiment comprises cyclosporin A, Lipowax D®, and Cinnamon oil.

Another embodiment comprises cyclosporin A, Lipowax G®, and Cinnamon oil.

Another embodiment comprises cyclosporin A, Lipowax NI®, and Cinnamon oil.

Another embodiment comprises cyclosporin A, Lipowax P® and Cinnamon oil.

Another embodiment comprises cyclosporin A, Lipowax P-31®, and Cinnamon oil.

Another embodiment comprises cyclosporin A, Lipowax PR®, and Cinnamon oil.

Another embodiment comprises cyclosporin A, Lumulse CS-20®, and Cinnamon oil.

Another embodiment comprises cyclosporin A, Macol CSA-20®, and Cinnamon oil.

Another embodiment comprises cyclosporin A, Mariox B 24/50®, and Cinnamon oil.

Another embodiment comprises cyclosporin A, Mazawet 77®, and Cinnamon oil.

Another embodiment comprises cyclosporin A, Norfox 1713®, and Cinnamon oil.

Another embodiment comprises cyclosporin A, Norfox 2579®, and Cinnamon oil.

Another embodiment comprises cyclosporin A, Norfox Lo Foam®, and Cinnamon oil.

Another embodiment comprises cyclosporin A, Promulgen D®, and Cinnamon oil.

Another embodiment comprises cyclosporin A, Promulgen G®, and Cinnamon oil.

Another embodiment comprises cyclosporin A, Renex 30®, and Cinnamon oil.

Another embodiment comprises cyclosporin A, Renex 36®, and Cinnamon oil.

Another embodiment comprises cyclosporin A, Rhodasurf A 24®, and Cinnamon oil.

Another embodiment comprises cyclosporin A, Rhodasurf AAE-10®, and Cinnamon oil.

Another embodiment comprises cyclosporin A, Rhodasurf BEH-25®, and Cinnamon oil.

Another embodiment comprises cyclosporin A, Rhodasurf BEH-40®, and Cinnamon oil.

Another embodiment comprises cyclosporin A, Rhodasurf DA 530®, and Cinnamon oil.

Another embodiment comprises cyclosporin A, Rhodasurf DA 630®, and Cinnamon oil.

Another embodiment comprises cyclosporin A, Rhodasurf DA 639®, and Cinnamon oil.

Another embodiment comprises cyclosporin A, Rhodasurf LAN-23®, and Cinnamon oil.

Another embodiment comprises cyclosporin A, Rhodasurf ON-870®, and Cinnamon oil.

Another embodiment comprises cyclosporin A, Rhodasurf ON-877®, and Cinnamon oil.

Another embodiment comprises cyclosporin A, Rhodasurf TB-970 FLK®, and Cinnamon oil.

Another embodiment comprises cyclosporin A, Ritacet-20®, and Cinnamon oil.

Another embodiment comprises cyclosporin A, Ritachol 1000®, and Cinnamon oil.

Another embodiment comprises cyclosporin A, Ritachol 2000®, and Cinnamon oil.

Another embodiment comprises cyclosporin A, Ritachol 5000®, and Cinnamon oil.

Another embodiment comprises cyclosporin A, Ritox 35®, and Cinnamon oil.

Another embodiment comprises cyclosporin A, Surfonic DA-4®, and Cinnamon oil.

Another embodiment comprises cyclosporin A, Surfonic DA-6®, and Cinnamon oil.

Another embodiment comprises cyclosporin A, Surfonic L46-7®, and Cinnamon oil.

Another embodiment comprises cyclosporin A, Surfonic POA®, and Cinnamon oil.

Another embodiment comprises cyclosporin A, Synthrapol KB®, and Cinnamon oil.

Another embodiment comprises cyclosporin A, Teginacid®, and Cinnamon oil.

Another embodiment comprises cyclosporin A, Teginacid C®, and Cinnamon oil.

Another embodiment comprises cyclosporin A, Tegotens EC 11®, and Cinnamon oil.

Another embodiment comprises cyclosporin A, Tinegal NA®, and Cinnamon oil.

Another embodiment comprises cyclosporin A, Tomadol 400®, and Cinnamon oil.

Another embodiment comprises cyclosporin A, Tomadol 600®, and Cinnamon oil.

Another embodiment comprises cyclosporin A, Tomadol 900®, and Cinnamon oil.

Another embodiment comprises cyclosporin A, Uniperol O®, and Cinnamon oil.

Another embodiment comprises cyclosporin A, Witconol SN Series®, and Cinnamon oil.

Another embodiment comprises cyclosporin A, an Ethoxylated Alkylphenol, and Cinnamon oil.

Another embodiment comprises cyclosporin A, Antarox LF-222®, and Cinnamon oil.

Another embodiment comprises cyclosporin A, Atlox 775®, and Cinnamon oil.

Another embodiment comprises cyclosporin A, Caloxylate N-9®, and Cinnamon oil.

Another embodiment comprises cyclosporin A, Canasol NF-1000®, and Cinnamon oil.

Another embodiment comprises cyclosporin A, Canasol NF-3000®, and Cinnamon oil.

Another embodiment comprises cyclosporin A, Canasol NF-3070®, and Cinnamon oil.

Another embodiment comprises cyclosporin A, Canasol OF 1670®, and Cinnamon oil.

Another embodiment comprises cyclosporin A, Canasol OF 2570®, and Cinnamon oil.

Another embodiment comprises cyclosporin A, Canasbl OF 4070®, and Cinnamon oil.

Another embodiment comprises cyclosporin A, Chemax DNP-8®, and Cinnamon oil.

Another embodiment comprises cyclosporin A, Chemax DNP-18®, and Cinnamon oil.

Another embodiment comprises cyclosporin A, Chemax DNP-150/50®, and Cinnamon oil.

Another embodiment comprises cyclosporin A, DeSonic 1.5N®, and Cinnamon oil.

Another embodiment comprises cyclosporin A, DeSonic 4N®, and Cinnamon oil.

Another embodiment comprises cyclosporin A, DeSonic 5N®, and Cinnamon oil.

Another embodiment comprises cyclosporin A, DeSonic 6D®, and Cinnamon oil.

Another embodiment comprises cyclosporin A, DeSonic 6N®, and Cinnamon oil.

Another embodiment comprises cyclosporin A, DeSonic 7N®, and Cinnamon oil.

Another embodiment comprises cyclosporin A, DeSonic 9N®, and Cinnamon oil.

Another embodiment comprises cyclosporin A, DeSonic 10D, and Cinnamon oil.

Another embodiment comprises cyclosporin A, DeSonic 11N®, and Cinnamon oil.

Another embodiment comprises cyclosporin A, DeSonic 12N®, and Cinnamon oil.

Another embodiment comprises cyclosporin A, DeSonic 15N®, and Cinnamon oil.

Another embodiment comprises cyclosporin A, DeSonic 20N®, and Cinnamon oil.

Another embodiment comprises cyclosporin A, Eccoscour RC®, and Cinnamon oil.

Another embodiment comprises cyclosporin A, Eccoterge EO-100®, and Cinnamon oil.

Another embodiment comprises cyclosporin A, Emulsifier 632/90%®, and Cinnamon oil.

Another embodiment comprises cyclosporin A, Geronol AG-821®, and Cinnamon oil.

Another embodiment comprises cyclosporin A, Gradonic N-95®, and Cinnamon oil.

Another embodiment comprises cyclosporin A, Hetoxide NP-4®, and Cinnamon oil.

Another embodiment comprises cyclosporin A, Hetoxide NP-30®, and Cinnamon oil.

Another embodiment comprises cyclosporin A, Hostapal N-100®, and Cinnamon oil.

Another embodiment comprises cyclosporin A, Hostapal N-110®, and Cinnamon oil.

Another embodiment comprises cyclosporin A, Igepal CTA-639W®, and Cinnamon oil.

Another embodiment comprises cyclosporin A, Igepal DAP-9®, and Cinnamon oil.

Another embodiment comprises cyclosporin A, Igepal OD-410®, and Cinnamon oil.

Another embodiment comprises cyclosporin A, Igepal SS-837®, and Cinnamon oil.

Another embodiment comprises cyclosporin A, Lipocol NP-9 USP®, and Cinnamon oil.

Another embodiment comprises cyclosporin A, Macol DNP-10®, and Cinnamon oil.

Another embodiment comprises cyclosporin A, Marlophen NP 5®, and Cinnamon oil.

Another embodiment comprises cyclosporin A, Marlophen P 1®, and Cinnamon oil.

Another embodiment comprises cyclosporin A, Surfonic NB®, and Cinnamon oil.

Another embodiment comprises cyclosporin A, Surfonic OPB-307®, and Cinnamon oil.

Another embodiment comprises cyclosporin A, Surfonic OPB-407®, and Cinnamon oil.

Another embodiment comprises cyclosporin A, Syn Fac 334®, and Cinnamon oil.

Another embodiment comprises cyclosporin A, Syn Fac 8216®, and Cinnamon oil.

Another embodiment comprises cyclosporin A, Triton N-57®, and Cinnamon oil.

Another embodiment comprises cyclosporin A, Trycol 6956®, and Cinnamon oil.

Another embodiment comprises cyclosporin A, Trycol 6961®, and Cinnamon oil.

Another embodiment comprises cyclosporin A, Trycol 6964®, and Cinnamon oil.

Another embodiment comprises cyclosporin A, Trycol 6969®, and Cinnamon oil.

Another embodiment comprises cyclosporin A, Trycol 6974®, and Cinnamon oil.

Another embodiment comprises cyclosporin A, Witbreak DRB-127®, and Cinnamon oil.

Another embodiment comprises cyclosporin A, Witbreak DRB-127®, and Cinnamon oil.

Another embodiment comprises cyclosporin A, Witconol NP Series®, and Cinnamon oil.

Another embodiment comprises cyclosporin A, an Ethoxylated Aryl Phenol. and Cinnamon oil.

Another embodiment comprises cyclosporin A, Soprophor BSU®, and Cinnamon oil.

Another embodiment comprises cyclosporin A, Soprophor CY/8®, and Cinnamon oil.

Another embodiment comprises cyclosporin A, Soprophor S/25®, and Cinnamon oil.

Another embodiment comprises cyclosporin A, Witconol NIO®, and Cinnamon oil.

Another embodiment comprises cyclosporin A, Witconol NIW®, and Cinnamon oil.

Another embodiment comprises cyclosporin A, Witconol S-100®, and Cinnamon oil.

Another embodiment comprises cyclosporin A, an Ethoxylated Fatty Acid, and Cinnamon oil.

Another embodiment comprises cyclosporin A, Aldo PGHMS®, and Cinnamon oil.

Another embodiment comprises cyclosporin A, Alkamuls TO-15/HR®, and Cinnamon oil.

Another embodiment comprises cyclosporin A, Armotan AL-69-66®, and Cinnamon oil.

Another embodiment comprises cyclosporin A, Cerasynt 840®, and Cinnamon oil.

Another embodiment comprises cyclosporin A, Cerasynt 945®, and Cinnamon oil.

Another embodiment comprises cyclosporin A, Crystal Inhibitor No. 5®, and Cinnamon oil.

Another embodiment comprises cyclosporin A, DeThox Acid L-9®, and Cinnamon oil.

Another embodiment comprises cyclosporin A, DeThox Acid S-8®, and Cinnamon oil.

Another embodiment comprises cyclosporin A, Ethofat 242/25®, and Cinnamon oil.

Another embodiment comprises cyclosporin A, Hydropalat 65®, and Cinnamon oil.

Another embodiment comprises cyclosporin A, Lipo EGMS®, and Cinnamon oil.

Another embodiment comprises cyclosporin A, Lipopeg 2 DL®, and Cinnamon oil.

Another embodiment comprises cyclosporin A, Lipopeg 4 DL®, and Cinnamon oil.

Another embodiment comprises cyclosporin A, Lipopeg 4-L®, and Cinnamon oil.

Another embodiment comprises cyclosporin A, Lipopeg 39-S®, and Cinnamon oil.

Another embodiment comprises cyclosporin A, Lipopeg 4-S®, and Cinnamon oil.

Another embodiment comprises cyclosporin A, Lipopeg 10-S®, and Cinnamon oil.

Another embodiment comprises cyclosporin A, Lipopeg 100-S®, and Cinnamon oil.

Another embodiment comprises cyclosporin A, Lipopeg 6000 DS®, and Cinnamon oil.

Another embodiment comprises cyclosporin A, Lumulse 40-L®, and Cinnamon oil.

Another embodiment comprises cyclosporin A, Lumulse 40-S®, and Cinnamon oil.

Another embodiment comprises cyclosporin A, Lumulse 42-L®, and Cinnamon oil.

Another embodiment comprises cyclosporin A, Lumulse 42-S®, and Cinnamon oil.

Another embodiment comprises cyclosporin A, Lumulse 100-S®, and Cinnamon oil.

Another embodiment comprises cyclosporin A, Lumulse 602-S®, and Cinnamon oil.

Another embodiment comprises cyclosporin A, Magrabar PGE-20-0®, and Cinnamon oil.

Another embodiment comprises cyclosporin A, Magrabar PGE-20L®, and Cinnamon oil.

Another embodiment comprises cyclosporin A, Magrabar PGE-20T®, and Cinnamon oil.

Another embodiment comprises cyclosporin A, Magrabar PGE-22-0®, and Cinnamon oil.

Another embodiment comprises cyclosporin A, Magrabar PGE-22L®, and Cinnamon oil.

Another embodiment comprises cyclosporin A, Magrabar PGE-22T®, and Cinnamon oil.

Another embodiment comprises cyclosporin A, Magrabar PGE-40-0®, and Cinnamon oil.

Another embodiment comprises cyclosporin A, Magrabar PGE-40L®, and Cinnamon oil.

Another embodiment comprises cyclosporin A, Magrabar PGE-40T®, and Cinnamon oil.

Another embodiment comprises cyclosporin A, Magrabar PGE-42-0®, and Cinnamon oil.

Another embodiment comprises cyclosporin A, Magrabar PGE-42L®, and Cinnamon oil.

Another embodiment comprises cyclosporin A, Magrabar PGE-42T®, and Cinnamon oil.

Another embodiment comprises cyclosporin A, Magrabar PGE-60-0®, and Cinnamon oil.

Another embodiment comprises cyclosporin A, Magrabar PGE-60L®, and Cinnamon oil.

Another embodiment comprises cyclosporin A, Magrabar PGE-60T®, and Cinnamon oil.

Another embodiment comprises cyclosporin A, Magrabar PGE-62-0®, and Cinnamon oil.

Another embodiment comprises cyclosporin A, Magrabar PGE-62L®, and Cinnamon oil.

Another embodiment comprises cyclosporin A, Magrabar PGE-62T®, and Cinnamon oil.

Another embodiment comprises cyclosporin A, Mapeg S-40K®, and Cinnamon oil.

Another embodiment comprises cyclosporin A, Marlowet OTS®, and Cinnamon oil.

Another embodiment comprises cyclosporin A, Naturechem PGR®, and Cinnamon oil.

Another embodiment comprises cyclosporin A, PG No. 4®, and Cinnamon oil.

Another embodiment comprises cyclosporin A, Renex 20®, and Cinnamon oil.

Another embodiment comprises cyclosporin A, Ritox 52®, and Cinnamon oil.

Another embodiment comprises cyclosporin A, Ritox 53®, and Cinnamon oil.

Another embodiment comprises cyclosporin A, Ritox 59®, and Cinnamon oil.

Another embodiment comprises cyclosporin A, Surfax 8916/A®, and Cinnamon oil.

Another embodiment comprises cyclosporin A, Tego Acid S 40 P® and Cinnamon oil.

Another embodiment comprises cyclosporin A, Tego Acid S 100 P®, and Cinnamon oil.

Another embodiment comprises cyclosporin A, Tween 20®, and Cinnamon oil.

Another embodiment comprises cyclosporin A, Volpo 131®, and Cinnamon oil.

Another embodiment comprises cyclosporin A, an Ethoxylated Fatty Ester or Oil, and Cinnamon oil.

Another embodiment comprises cyclosporin A, Acconon 6-C10®, and Cinnamon oil.

Another embodiment comprises cyclosporin A, Acconon CC-6®, and Cinnamon oil.

Another embodiment comprises cyclosporin A, Acconon CO-7®, and Cinnamon oil.

Another embodiment comprises cyclosporin A, Aldosperse 40/60 FG®, and Cinnamon oil.

Another embodiment comprises cyclosporin A, Aldosperse ML-23®, and Cinnamon oil.

Another embodiment comprises cyclosporin A, Aldosperse MS-20 FG®, and Cinnamon oil.

Another embodiment comprises cyclosporin A, Alkamuls EL-620®, and Cinnamon oil.

Another embodiment comprises cyclosporin A, Alkamuls EL-719®, and Cinnamon oil.

Another embodiment comprises cyclosporin A, Alkamuls EL-985®, and Cinnamon oil.

Another embodiment comprises cyclosporin A, Arlatone G®, and Cinnamon oil.

Another embodiment comprises cyclosporin A, Arlatone T®, and Cinnamon oil.

Another embodiment comprises cyclosporin A, Atlas G-1045A®, and Cinnamon oil.

Another embodiment comprises cyclosporin A, Atlas G-1086®, and Cinnamon oil.

Another embodiment comprises cyclosporin A, Atlas G-1087®, and Cinnamon oil.

Another embodiment comprises cyclosporin A, Atlas G-1089®, and Cinnamon oil.

Another embodiment comprises cyclosporin A, Atlas G-1096®, and Cinnamon oil.

Another embodiment comprises cyclosporin A, Atlas G-1292®, and Cinnamon oil.

Another embodiment comprises cyclosporin A, Atlas G-1293®, and Cinnamon oil.

Another embodiment comprises cyclosporin A, Atlas G-1300®, and Cinnamon oil.

Another embodiment comprises cyclosporin A, Atlas G-7076®, and Cinnamon oil.

Another embodiment comprises cyclosporin A, Capmul EMG®, and Cinnamon oil.

Another embodiment comprises cyclosporin A, Chemonic CO-40®, and Cinnamon oil.

Another embodiment comprises cyclosporin A, Chemonic LI-3®, and Cinnamon oil.

Another embodiment comprises cyclosporin A, Chemonic LI-7®, and Cinnamon oil.

Another embodiment comprises cyclosporin A, Cirrasol GM®, and Cinnamon oil.

Another embodiment comprises cyclosporin A, Cremophor CO 40®, and Cinnamon oil.

Another embodiment comprises cyclosporin A, Cremophor CO 410®, and Cinnamon oil.

Another embodiment comprises cyclosporin A, Cremophor EL®, and Cinnamon oil.

Another embodiment comprises cyclosporin A, Cremophor GC7®, and Cinnamon oil.

Another embodiment comprises cyclosporin A, Cremophor RH-40®, and Cinnamon oil.

Another embodiment comprises cyclosporin A, Crovol A-40®, and Cinnamon oil.

Another embodiment comprises cyclosporin A, Crovol A-70®, and Cinnamon oil.

Another embodiment comprises cyclosporin A, Crovol M-70®, and Cinnamon oil.

Another embodiment comprises cyclosporin A, Crovol PK-70®, and Cinnamon oil.

Another embodiment comprises cyclosporin A, Cutina E-24®, and Cinnamon oil.

Another embodiment comprises cyclosporin A, Dacospin 12-R®, and Cinnamon oil.

Another embodiment comprises cyclosporin A, Dehymuls HRE-7®, and Cinnamon oil.

Another embodiment comprises cyclosporin A, DeSonic 30C®, and Cinnamon oil.

Another embodiment comprises cyclosporin A, DeSonic 36C®, and Cinnamon oil.

Another embodiment comprises cyclosporin A, DeSonic 40C®, and Cinnamon oil.

Another embodiment comprises cyclosporin A, Durfax 60®, and Cinnamon oil.

Another embodiment comprises cyclosporin A, Durfax 65®, and Cinnamon oil.

Another embodiment comprises cyclosporin A, Durfax 80®, and Cinnamon oil.

Another embodiment comprises cyclosporin A, Durfax EOM®, and Cinnamon oil.

Another embodiment comprises cyclosporin A, Eccoterge NF-2®, and Cinnamon oil.

Another embodiment comprises cyclosporin A, Emulpon CO-360®, and Cinnamon oil.

Another embodiment comprises cyclosporin A, Emulpon CO-550®, and Cinnamon oil.

Another embodiment comprises cyclosporin A, Emulsogen EL®, and Cinnamon oil.

Another embodiment comprises cyclosporin A, Emulsogen HCO 040®, and Cinnamon oil.

Another embodiment comprises cyclosporin A, Emulsogen HCO 060®, and Cinnamon oil.

Another embodiment comprises cyclosporin A, Emulsynt 1055®, and Cinnamon oil.

Another embodiment comprises cyclosporin A, Ethox 3095®, and Cinnamon oil.

Another embodiment comprises cyclosporin A, Eumulgin RO-40®, and Cinnamon oil.

Another embodiment comprises cyclosporin A, Genapol G-260®, and Cinnamon oil.

Another embodiment comprises cyclosporin A, Glycosperse L-20®, and Cinnamon oil.

Another embodiment comprises cyclosporin A, Glycosperse O-5®, and Cinnamon oil.

Another embodiment comprises cyclosporin A, Glycosperse O-20®, and Cinnamon oil.

Another embodiment comprises cyclosporin A, Glycosperse O-20 FG®, and Cinnamon oil.

Another embodiment comprises cyclosporin A, Glycosperse S-20®, and Cinnamon oil.

Another embodiment comprises cyclosporin A, Glycosperse S-20 FG®, and Cinnamon oil.

Another embodiment comprises cyclosporin A, Glycosperse TS-20®, and Cinnamon oil.

Another embodiment comprises cyclosporin A, Glycosperse TS-20 FG®, and Cinnamon oil.

Another embodiment comprises cyclosporin A, Hetan SL®, and Cinnamon oil.

Another embodiment comprises cyclosporin A, Hetan SO®, and Cinnamon oil.

Another embodiment comprises cyclosporin A, Hetan SS®, and Cinnamon oil.

Another embodiment comprises cyclosporin A, Hetoxide C-2®, and Cinnamon oil.

Another embodiment comprises cyclosporin A, Hetoxide C-9®, and Cinnamon oil.

Another embodiment comprises cyclosporin A, Hetoxide C-15®, and Cinnamon oil.

Another embodiment comprises cyclosporin A, Hetoxide C-25®, and Cinnamon oil.

Another embodiment comprises cyclosporin A, Hetoxide C-40®, and Cinnamon oil.

Another embodiment comprises cyclosporin A, Hetoxide C-200®, and Cinnamon oil.

Another embodiment comprises cyclosporin A, Hetoxide C-200-50%®, and Cinnamon oil.

Another embodiment comprises cyclosporin A, Hetoxide GC-30®, and Cinnamon oil.

Another embodiment comprises cyclosporin A, Hetoxide HC-60®, and Cinnamon oil.

Another embodiment comprises cyclosporin A, Ice No. 2®, and Cinnamon oil.

Another embodiment comprises cyclosporin A, Incrocas 30/40®, and Cinnamon oil.

Another embodiment comprises cyclosporin A, Lexol EC®, and Cinnamon oil.

Another embodiment comprises cyclosporin A, Lexol EO®, and Cinnamon oil.

Another embodiment comprises cyclosporin A, Lipocol HCO-40®, and Cinnamon oil.

Another embodiment comprises cyclosporin A, Lipocol HCO-60®, and Cinnamon oil.

Another embodiment comprises cyclosporin A, Lipocol O-3 Special®, and Cinnamon oil.

Another embodiment comprises cyclosporin A, Lipopeg 2-L®, and Cinnamon oil.

Another embodiment comprises cyclosporin A, Lipopeg 4-DO®, and Cinnamon oil.

Another embodiment comprises cyclosporin A, Lipopeg 4-DS®, and Cinnamon oil.

Another embodiment comprises cyclosporin A, Lipovol GTB®, and Cinnamon oil.

Another embodiment comprises cyclosporin A, Lonzest SML-20®, and Cinnamon oil.

Another embodiment comprises cyclosporin A, Lonzest SMO-20®, and Cinnamon oil.

Another embodiment comprises cyclosporin A, Lonzest SMS-20®, and Cinnamon oil.

Another embodiment comprises cyclosporin A, Lonzest STO-208, and Cinnamon oil.

Another embodiment comprises cyclosporin A, Lonzest STS-20®, and Cinnamon oil.

Another embodiment comprises cyclosporin A, Lumulse GR-40®, and Cinnamon oil.

Another embodiment comprises cyclosporin A, Lumulse GRH-40®, and Cinnamon oil.

Another embodiment comprises cyclosporin A, Lumulse POE (7) GML®, and Cinnamon oil.

Another embodiment comprises cyclosporin A, Lumulse POE (12) Glyc®, and Cinnamon oil.

Another embodiment comprises cyclosporin A, Lumulse POE (40) MS KF®, and Cinnamon oil.

Another embodiment comprises cyclosporin A, Marlowet 4750®, and Cinnamon oil.

Another embodiment comprises cyclosporin A, Marlowet LVS®, and Cinnamon oil.

Another embodiment comprises cyclosporin A, Marlowet R 11®, and Cinnamon oil.

Another embodiment comprises cyclosporin A, Marlowet R 40®, and Cinnamon oil.

Another embodiment comprises cyclosporin A, Mazol 80 MGK®, and Cinnamon oil.

Another embodiment comprises cyclosporin A, Nonionic Emulsifier T-9®, and Cinnamon oil.

Another embodiment comprises cyclosporin A, Oronal LCG®, and Cinnamon oil.

Another embodiment comprises cyclosporin A, Polyderm PPI-CO-200®, and Cinnamon oil.

Another embodiment comprises cyclosporin A, Polyderm PPI-CO-40®, and Cinnamon oil.

Another embodiment comprises cyclosporin A, Rewoderm LI 520-70®, and Cinnamon oil.

Another embodiment comprises cyclosporin A, Ritapeg 150 DS®, and Cinnamon oil.

Another embodiment comprises cyclosporin A, Softigen 767®, and Cinnamon oil.

Another embodiment comprises cyclosporin A, Surfactol 318®, and Cinnamon oil.

Another embodiment comprises cyclosporin A, Surfactol 365®, and Cinnamon oil.

Another embodiment comprises cyclosporin A, Syn Lube 107®, and Cinnamon oil.

Another embodiment comprises cyclosporin A, Syn Lube 728®, and Cinnamon oil.

Another embodiment comprises cyclosporin A, Syn Lube 1632H®, and Cinnamon oil.

Another embodiment comprises cyclosporin A, Syn Lube 6277-A®, and Cinnamon oil.

Another embodiment comprises cyclosporin A, T-Det C-20®, and Cinnamon oil.

Another embodiment comprises cyclosporin A, T-Det C-40®, and Cinnamon oil.

Another embodiment comprises cyclosporin A, Tally 100 Plus®, and Cinnamon oil.

Another embodiment comprises cyclosporin A, Uniperol EL®, and Cinnamon oil.

Another embodiment comprises cyclosporin A, a Fatty Ester, and Cinnamon oil.

Another embodiment comprises cyclosporin A, Actralube-Syn 147®, and Cinnamon oil.

Another embodiment comprises cyclosporin A, Atlas G-1556®, and Cinnamon oil.

Another embodiment comprises cyclosporin A, Atlas G-1564®, and Cinnamon oil.

Another embodiment comprises cyclosporin A, Atlasol Base Oil S®, and Cinnamon oil.

Another embodiment comprises cyclosporin A, Base ML®, and Cinnamon oil.

Another embodiment comprises cyclosporin A, Base MT®, and Cinnamon oil.

Another embodiment comprises cyclosporin A, Cerasynt 303®, and Cinnamon oil.

Another embodiment comprises cyclosporin A, Dermol 1012®, and Cinnamon oil.

Another embodiment comprises cyclosporin A, Kemester 4000®, and Cinnamon oil.

Another embodiment comprises cyclosporin A, Lactipol S®, and Cinnamon oil.

Another embodiment comprises cyclosporin A, Magrabar PGO®, and Cinnamon oil.

Another embodiment comprises cyclosporin A, Mayco Base BFO®, and Cinnamon oil.

Another embodiment comprises cyclosporin A, Methyl Linoleate®, and Cinnamon oil.

Another embodiment comprises cyclosporin A, Pationic 122A®, and Cinnamon oil.

Another embodiment comprises cyclosporin A, Pationic 138C®, and Cinnamon oil.

Another embodiment comprises cyclosporin A, Pationic CSL®, and Cinnamon oil.

Another embodiment comprises cyclosporin A, Pationic ISL®, and Cinnamon oil.

Another embodiment comprises cyclosporin A, Pationic SBL®, and Cinnamon oil..

Another embodiment comprises cyclosporin A, Pationic SSL®, and Cinnamon oil.

Another embodiment comprises cyclosporin A, Ritasol®, and Cinnamon oil.

Another embodiment comprises cyclosporin A, Tego Alkanol CS 20®, and Cinnamon oil.

Another embodiment comprises cyclosporin A, Tego Alkanol L23 P® and Cinnamon oil.

Another embodiment comprises cyclosporin A, Tego Alkanol S2®, and Cinnamon oil.

Another embodiment comprises cyclosporin A, Tego Alkanol S20 P®, and Cinnamon oil.

Another embodiment comprises cyclosporin A, Triemulsifier 600 MS®, and Cinnamon oil.

Another embodiment comprises cyclosporin A, a Glycerol Esters, and Cinnamon oil.

Another embodiment comprises cyclosporin A, Agro #9 Wint SBO®, and Cinnamon oil.

Another embodiment comprises cyclosporin A, Ahcovel Base 700®, and Cinnamon oil.

Another embodiment comprises cyclosporin A, Aldo HMS FG®, and Cinnamon oil.

Another embodiment comprises cyclosporin A, Aldo MLD®, and Cinnamon oil.

Another embodiment comprises cyclosporin A, Aldo MLD FG®, and Cinnamon oil.

Another embodiment comprises cyclosporin A, Aldo MO FG®, and Cinnamon oil.

Another embodiment comprises cyclosporin A, Aldo MS®, and Cinnamon oil.

Another embodiment comprises cyclosporin A, Aldo MS FG®, and Cinnamon oil.

Another embodiment comprises cyclosporin A, Aldo MS LG FG®, and Cinnamon oil.

Another embodiment comprises cyclosporin A, Aldo MSD®, and Cinnamon oil.

Another embodiment comprises cyclosporin A, Aldo MSD FG®, and Cinnamon oil.

Another embodiment comprises cyclosporin A, Aldosperse O-20 FG®, and Cinnamon oil.

Another embodiment comprises cyclosporin A, Aldosperse TS-20 FG®, and Cinnamon oil.

Another embodiment comprises cyclosporin A, Aldosperse TS-40 FG®, and Cinnamon oil.

Another embodiment comprises cyclosporin A, Arlacel 165®, and Cinnamon oil.

Another embodiment comprises cyclosporin A, Arlacel 186®, and Cinnamon oil.

Another embodiment comprises cyclosporin A, Capmul GMO®, and Cinnamon oil.

Another embodiment comprises cyclosporin A, Capmul GMS®, and Cinnamon oil.

Another embodiment comprises cyclosporin A, Caprol 3GO®, and Cinnamon oil.

Another embodiment comprises cyclosporin A, Caprol 3GVS®, and Cinnamon oil.

Another embodiment comprises cyclosporin A, Caprol 6G2S®, and Cinnamon oil.

Another embodiment comprises cyclosporin A, Caprol 10G40®, and Cinnamon oil.

Another embodiment comprises cyclosporin A, Caprol 10G100®, and Cinnamon oil.

Another embodiment comprises cyclosporin A, Caprol ET®, and Cinnamon oil.

Another embodiment comprises cyclosporin A, Caprol PGE860®, and Cinnamon oil.

Another embodiment comprises cyclosporin A, Cerasynt 945®, and Cinnamon oil.

Another embodiment comprises cyclosporin A, Cerasynt GMS®, and Cinnamon oil.

Another embodiment comprises cyclosporin A, Cerasynt Q®, and Cinnamon oil..

Another embodiment comprises cyclosporin A, Cerasynt SD®, and Cinnamon oil.

Another embodiment comprises cyclosporin A, Cerasynt WM®, and Cinnamon oil.

Another embodiment comprises cyclosporin A, Chemsperse 14®, and Cinnamon oil.

Another embodiment comprises cyclosporin A, Cremophor GO-32®, and Cinnamon oil.

Another embodiment comprises cyclosporin A, Cremophor GS11®, and Cinnamon oil.

Another embodiment comprises cyclosporin A, Cremophor GS-32®, and Cinnamon oil.

Another embodiment comprises cyclosporin A, Cutina KD-16®, and Cinnamon oil.

Another embodiment comprises cyclosporin A, Dehymuls PGPH®, and Cinnamon oil.

Another embodiment comprises cyclosporin A, Dermol DGDIS®, and Cinnamon oil.

Another embodiment comprises cyclosporin A, Dermol DGMIS®, and Cinnamon oil.

Another embodiment comprises cyclosporin A, Dermol G-76®, and Cinnamon oil.

Another embodiment comprises cyclosporin A, Dermol G-7DI®, and Cinnamon oil.

Another embodiment comprises cyclosporin A, Dermol NGDI®, and Cinnamon oil.

Another embodiment comprises cyclosporin A, Dermolan GLH®, and Cinnamon oil.

Another embodiment comprises cyclosporin A, Drewmulse GMO®, and Cinnamon oil.

Another embodiment comprises cyclosporin A, Drewpol 3-5-M®, and Cinnamon oil.

Another embodiment comprises cyclosporin A, Durlac 100 W®, and Cinnamon oil.

Another embodiment comprises cyclosporin A, Dur-Lo®, and Cinnamon oil.

Another embodiment comprises cyclosporin A, Dynasan 118®, and Cinnamon oil.

Another embodiment comprises cyclosporin A, EC-25®, and Cinnamon oil.

Another embodiment comprises cyclosporin A, EM 40®, and Cinnamon oil.

Another embodiment comprises cyclosporin A, Emerest 2400®, and Cinnamon oil.

Another embodiment comprises cyclosporin A, Emerest 2452®, and Cinnamon oil.

Another embodiment comprises cyclosporin A, Empilan G-26®, and Cinnamon oil.

Another embodiment comprises cyclosporin A, Genapol TSM®, and Cinnamon oil.

Another embodiment comprises cyclosporin A, Hostacerin DGI®, and Cinnamon oil.

Another embodiment comprises cyclosporin A, Hostacerin DGL®, and Cinnamon oil.

Another embodiment comprises cyclosporin A, Hostacerin DGMS®, and Cinnamon oil.

Another embodiment comprises cyclosporin A, Hostacerin DGSB®, and Cinnamon oil.

Another embodiment comprises cyclosporin A, Ice No. 2®, and Cinnamon oil.

Another embodiment comprises cyclosporin A, Imwitor 742®, and Cinnamon oil.

Another embodiment comprises cyclosporin A, Imwitor 780 K®, and Cinnamon oil.

Another embodiment comprises cyclosporin A, Imwitor 960 Flakes®, and Cinnamon oil.

Another embodiment comprises cyclosporin A, Isolan GI 34®, and Cinnamon oil.

Another embodiment comprises cyclosporin A, Isolan GO 33®, and Cinnamon oil.

Another embodiment comprises cyclosporin A, Kemester 1000®, and Cinnamon oil.

Another embodiment comprises cyclosporin A, Kemester 2000®, and Cinnamon oil.

Another embodiment comprises cyclosporin A, Kemester 2000®, and Cinnamon oil.

Another embodiment comprises cyclosporin A, Kemester 6000SE®, and Cinnamon oil.

Another embodiment comprises cyclosporin A, Lamecreme DGE 18®, and Cinnamon oil.

Another embodiment comprises cyclosporin A, Lexemul 515®, and Cinnamon oil.

Another embodiment comprises cyclosporin A, Lexemul 561®, and Cinnamon oil.

Another embodiment comprises cyclosporin A, Lexemul AR®, and Cinnamon oil.

Another embodiment comprises cyclosporin A, Lexemul AS®, and Cinnamon oil.

Another embodiment comprises cyclosporin A, Lexemul GDL®, and Cinnamon oil.

Another embodiment comprises cyclosporin A, Lexemul T®, and Cinnamon oil.

Another embodiment comprises cyclosporin A, Lipomulse 165®, and Cinnamon oil.

Another embodiment comprises cyclosporin A, Lumulse GML K®, and Cinnamon oil.

Another embodiment comprises cyclosporin A, Lumulse GMO K®, and Cinnamon oil.

Another embodiment comprises cyclosporin A, Lumulse GMR K®, and Cinnamon oil.

Another embodiment comprises cyclosporin A, Lumulse GMT K®, and Cinnamon oil.

Another embodiment comprises cyclosporin A, Magrabar GMC®, and Cinnamon oil.

Another embodiment comprises cyclosporin A, Magrabar GMO-CK®, and Cinnamon oil.

Another embodiment comprises cyclosporin A, Magrabar MDG-5050®, and Cinnamon oil.

Another embodiment comprises cyclosporin A, Magrabar PGO-315®, and Cinnamon oil.

Another embodiment comprises cyclosporin A, Magrabar PGO-1010®, and Cinnamon oil.

Another embodiment comprises cyclosporin A, Mazol 300K®, and Cinnamon oil.

Another embodiment comprises cyclosporin A, Mazol GMO-K®, and Cinnamon oil.

Another embodiment comprises cyclosporin A, Mazol GMS-K®, and Cinnamon oil.

Another embodiment comprises cyclosporin A, Mazol PG031-K®, and Cinnamon oil.

Another embodiment comprises cyclosporin A, Miglyol 812®, and Cinnamon oil.

Another embodiment comprises cyclosporin A, Norfox 165C®, and Cinnamon oil.

Another embodiment comprises cyclosporin A, Schercemol GMIS®, and Cinnamon oil.

Another embodiment comprises cyclosporin A, Tegin®, and Cinnamon oil.

Another embodiment comprises cyclosporin A, Tegin 4100 Pellets®, and Cinnamon oil.

Another embodiment comprises cyclosporin A, Tegin M Pellets®, and Cinnamon oil.

Another embodiment comprises cyclosporin A, Tegin OV®, and Cinnamon oil.

Another embodiment comprises cyclosporin A, Teginacid H®, and Cinnamon oil.

Another embodiment comprises cyclosporin A, Tego Cosmo P813®, and Cinnamon oil.

Another embodiment comprises cyclosporin A, Wickenol 535®, and Cinnamon oil.

Another embodiment comprises cyclosporin A, Witconol 14®, and Cinnamon oil.

Another embodiment comprises cyclosporin A, Witconol 14®, and Cinnamon oil.

Another embodiment comprises cyclosporin A, Witconol 14F®, and Cinnamon oil.

Another embodiment comprises cyclosporin A, Witconol 18®L, and Cinnamon oil.

Another embodiment comprises cyclosporin A, Witconol GOT®, and Cinnamon oil.

Another embodiment comprises cyclosporin A, Witconol MST®, and Cinnamon oil.

Another embodiment comprises cyclosporin A, Witconol RHT®, and Cinnamon oil.

Another embodiment comprises cyclosporin A, a Glycol Ester, and Cinnamon oil.

Another embodiment comprises cyclosporin A, Alkamuls 600 DO®, and Cinnamon oil.

Another embodiment comprises cyclosporin A, Alkamuls SEG®, and Cinnamon oil.

Another embodiment comprises cyclosporin A, Atlas EM-2®, and Cinnamon oil.

Another embodiment comprises cyclosporin A, Cerasynt IP®, and Cinnamon oil.

Another embodiment comprises cyclosporin A, Cerasynt M®, and Cinnamon oil.

Another embodiment comprises cyclosporin A, Cerasynt MN®, and Cinnamon oil.

Another embodiment comprises cyclosporin A, Cerasynt PA®, and Cinnamon oil.

Another embodiment comprises cyclosporin A, Chemsperse EGDS®, and Cinnamon oil.

Another embodiment comprises cyclosporin A, Chemsperse EGMS®, and Cinnamon oil.

Another embodiment comprises cyclosporin A, Colonial Monolaurin®, and Cinnamon oil.

Another embodiment comprises cyclosporin A, DeMuls SGE-95®, and Cinnamon oil.

Another embodiment comprises cyclosporin A, Eccoterge 200®, and Cinnamon oil.

Another embodiment comprises cyclosporin A, Emerest 2380®, and Cinnamon oil.

Another embodiment comprises cyclosporin A, Ethox 2610®, and Cinnamon oil.

Another embodiment comprises cyclosporin A, Ethox DO-9®, and Cinnamon oil.

Another embodiment comprises cyclosporin A, Ethox DO-14®, and Cinnamon oil.

Another embodiment comprises cyclosporin A, Ethox SO-9®, and Cinnamon oil.

Another embodiment comprises cyclosporin A, Fizul MD-318®, and Cinnamon oil.

Another embodiment comprises cyclosporin A, Genapol EGDS-VHP®, and Cinnamon oil.

Another embodiment comprises cyclosporin A, Genapol TS Powder®, and Cinnamon oil.

Another embodiment comprises cyclosporin A, Hostacerin WO®, and Cinnamon oil.

Another embodiment comprises cyclosporin A, Inversol 140®, and Cinnamon oil.

Another embodiment comprises cyclosporin A, Kemester 104®, and Cinnamon oil.

Another embodiment comprises cyclosporin A, Kemester 205®, and Cinnamon oil.

Another embodiment comprises cyclosporin A, Kemester 226®, and Cinnamon oil.

Another embodiment comprises cyclosporin A, Kemester 5221SE®, and Cinnamon oil.

Another embodiment comprises cyclosporin A, Kemester EGDS®, and Cinnamon oil.

Another embodiment comprises cyclosporin A, Lexemul EGDS®, and Cinnamon oil.

Another embodiment comprises cyclosporin A, Lexemul EGMS®, and Cinnamon oil.

Another embodiment comprises cyclosporin A, Lexemul P®, and Cinnamon oil.

Another embodiment comprises cyclosporin A, Lipo DGLS®, and Cinnamon oil.

Another embodiment comprises cyclosporin A, Lipo EGDS®, and Cinnamon oil.

Another embodiment comprises cyclosporin A, Lipo PGMS®, and Cinnamon oil.

Another embodiment comprises cyclosporin A, Liposorb S-4®, and Cinnamon oil.

Another embodiment comprises cyclosporin A, Liposorb TO-20®, and Cinnamon oil.

Another embodiment comprises cyclosporin A, Lumulse PGO®, and Cinnamon oil.

Another embodiment comprises cyclosporin A, Mackester EGDS®, and Cinnamon oil.

Another embodiment comprises cyclosporin A, Mackester EGMS®, and Cinnamon oil.

Another embodiment comprises cyclosporin A, Mackester GSTP®, and Cinnamon oil.

Another embodiment comprises cyclosporin A, Mackester Series®, and Cinnamon oil.

Another embodiment comprises cyclosporin A, Magrabar PDG-50®, and Cinnamon oil.

Another embodiment comprises cyclosporin A, Mapeg 6000 DS®, and Cinnamon oil.

Another embodiment comprises cyclosporin A, Marlowet 4702®, and Cinnamon oil.

Another embodiment comprises cyclosporin A, Monalube 305®, and Cinnamon oil.

Another embodiment comprises cyclosporin A, Monalube 310®, and Cinnamon oil.

Another embodiment comprises cyclosporin A, Monalube 315®, and Cinnamon oil.

Another embodiment comprises cyclosporin A, Monalube 320®, and Cinnamon oil.

Another embodiment comprises cyclosporin A, Monalube 325®, and Cinnamon oil.

Another embodiment comprises cyclosporin A, Monalube 330®, and Cinnamon oil.

Another embodiment comprises cyclosporin A, Naturechem PGHS®, and Cinnamon oil.

Another embodiment comprises cyclosporin A, Polycastorol PLO-840®, and Cinnamon oil.

Another embodiment comprises cyclosporin A, Polytex 10M®, and Cinnamon oil.

Another embodiment comprises cyclosporin A, Ritasynt IP®, and Cinnamon oil.

Another embodiment comprises cyclosporin A, Ross Chem PEG 600 DT®, and Cinnamon oil.

Another embodiment comprises cyclosporin A, Schercemol PGMS®, and Cinnamon oil.

Another embodiment comprises cyclosporin A, Sponto H-44C®, and Cinnamon oil.

Another embodiment comprises cyclosporin A, Tegin G®, and Cinnamon oil.

Another embodiment comprises cyclosporin A, Witbreak DGE-182®, and Cinnamon oil.

Another embodiment comprises cyclosporin A, Witbreak DGE-182®, and Cinnamon oil.

Another embodiment comprises cyclosporin A, Witbreak DRA-21®, and Cinnamon oil.

Another embodiment comprises cyclosporin A, Witbreak DRA-21®, and Cinnamon oil.

Another embodiment comprises cyclosporin A, Witbreak DRA-50®, and Cinnamon oil.

Another embodiment comprises cyclosporin A, Witbreak DRA-50®, and Cinnamon oil.

Another embodiment comprises cyclosporin A, Witconol F26-46®, and Cinnamon oil.

Another embodiment comprises cyclosporin A, Witconol H-32®, and Cinnamon oil.

Another embodiment comprises cyclosporin A, Witconol H-33®, and Cinnamon oil.

Another embodiment comprises cyclosporin A, Witconol H-35A®, and Cinnamon oil.

Another embodiment comprises cyclosporin A, Witconol RHP®, and Cinnamon oil.

Another embodiment comprises cyclosporin A, a Lanolin-based Derivative, and Cinnamon oil.

Another embodiment comprises cyclosporin A, Amerchol CAB®, and Cinnamon oil.

Another embodiment comprises cyclosporin A, Amerchol L-101®, and Cinnamon oil.

Another embodiment comprises cyclosporin A, Amerlate LFA-LO®, and Cinnamon oil.

Another embodiment comprises cyclosporin A, Amerlate P®, and Cinnamon oil.

Another embodiment comprises cyclosporin A, Barre Common Degras®, and Cinnamon oil.

Another embodiment comprises cyclosporin A, Cholesterol NF®, and Cinnamon oil.

Another embodiment comprises cyclosporin A, Crodalan AWS®, and Cinnamon oil.

Another embodiment comprises cyclosporin A, Crodalan LA®, and Cinnamon oil.

Another embodiment comprises cyclosporin A, Emery 1650®, and Cinnamon oil.

Another embodiment comprises cyclosporin A, Emery 1650®, and Cinnamon oil.

Another embodiment comprises cyclosporin A, Emery 1740®, and Cinnamon oil.

Another embodiment comprises cyclosporin A, Emery 1740®, and Cinnamon oil.

Another embodiment comprises cyclosporin A, Forlan 500®, and Cinnamon oil.

Another embodiment comprises cyclosporin A, Forlan L®, and Cinnamon oil.

Another embodiment comprises cyclosporin A, Laneto 50®, and Cinnamon oil.

Another embodiment comprises cyclosporin A, Laneto 100®, and Cinnamon oil.

Another embodiment comprises cyclosporin A, Laneto AWS®, and Cinnamon oil.

Another embodiment comprises cyclosporin A, Lanfrax 1776®, and Cinnamon oil.

Another embodiment comprises cyclosporin A, Lanfrax 1776®, and Cinnamon oil.

Another embodiment comprises cyclosporin A, Lanogel 21®, and Cinnamon oil.

Another embodiment comprises cyclosporin A, Lipolan®, and Cinnamon oil.

Another embodiment comprises cyclosporin A, Lipolan 31®, and Cinnamon oil.

Another embodiment comprises cyclosporin A, OHlan®, and Cinnamon oil.

Another embodiment comprises cyclosporin A, Polychol 5®, and Cinnamon oil.

Another embodiment comprises cyclosporin A, Polychol 15®, and Cinnamon oil.

Another embodiment comprises cyclosporin A, Ritacetyl®, and Cinnamon oil.

Another embodiment comprises cyclosporin A, Ritachol®, and Cinnamon oil.

Another embodiment comprises cyclosporin A, Ritahydrox®, and Cinnamon oil.

Another embodiment comprises cyclosporin A, Ritalafa®, and Cinnamon oil.

Another embodiment comprises cyclosporin A, Ritalan®, and Cinnamon oil.

Another embodiment comprises cyclosporin A, Ritalan AWS®, and Cinnamon oil.

Another embodiment comprises cyclosporin A, Ritalan C®, and Cinnamon oil.

Another embodiment comprises cyclosporin A, Ritawax®, and Cinnamon oil.

Another embodiment comprises cyclosporin A, Ritawax AEO®, and Cinnamon oil.

Another embodiment comprises cyclosporin A, Ritawax ALA®, and Cinnamon oil.

Another embodiment comprises cyclosporin A, Solan/Solan 50/Super Solan®, and Cinnamon oil.

Another embodiment comprises cyclosporin A, Super Hartolan/Hartolan®, and Cinnamon oil.

Another embodiment comprises cyclosporin A, Supersat AWS-4®, and Cinnamon oil.

Another embodiment comprises cyclosporin A, Supersat AWS-24®, and Cinnamon oil.

Another embodiment comprises cyclosporin A, a Lecithin or Lecithin Derivative, and Cinnamon oil.

Another embodiment comprises cyclosporin A, Alcolec®, and Cinnamon oil.

Another embodiment comprises cyclosporin A, Lecithin®, and Cinnamon oil.

Another embodiment comprises cyclosporin A, Lexin K®, and Cinnamon oil.

Another embodiment comprises cyclosporin A, Natipide®, and Cinnamon oil.

Another embodiment comprises cyclosporin A, a Lignin or Lignin Derivative. and Cinnamon oil.

Another embodiment comprises cyclosporin A, Diwatex XP 9®, and Cinnamon oil.

Another embodiment comprises cyclosporin A, Dynasperse LCD®, and Cinnamon oil.

Another embodiment comprises cyclosporin A, Indulin SAL®, and Cinnamon oil.

Another embodiment comprises cyclosporin A, Indulin W-1®, and Cinnamon oil.

Another embodiment comprises cyclosporin A, Indulin W-5®, and Cinnamon oil.

Another embodiment comprises cyclosporin A, Lignosol FTA®, and Cinnamon oil.

Another embodiment comprises cyclosporin A, Lignosol SFX-65®, and Cinnamon oil.

Another embodiment comprises cyclosporin A, Marasperse 52 CP®, and Cinnamon oil.

Another embodiment comprises cyclosporin A, Marasperse AG®, and Cinnamon oil.

Another embodiment comprises cyclosporin A, Marasperse CBOS-4®, and Cinnamon oil.

Another embodiment comprises cyclosporin A, Ufoxane 2®, and Cinnamon oil.

Another embodiment comprises cyclosporin A, a Methyl Ester, and Cinnamon oil.

Another embodiment comprises cyclosporin A, E.B. Cleaner AK®, and Cinnamon oil.

Another embodiment comprises cyclosporin A, Oleocal ME-70®, and Cinnamon oil.

Another embodiment comprises cyclosporin A, Trade Name Company®, and Cinnamon oil.

Another embodiment comprises cyclosporin A, Methyl Esters (cont'd)®, and Cinnamon oil.

Another embodiment comprises cyclosporin A, Oleocal ME-92®, and Cinnamon oil.

Another embodiment comprises cyclosporin A, Oleocal ME-112®, and Cinnamon oil.

Another embodiment comprises cyclosporin A, Oleocal ME-130®, and Cinnamon oil.

Another embodiment comprises cyclosporin A, a Monoglyceride or a Derivative thereof, and Cinnamon oil.

Another embodiment comprises cyclosporin A, Dynacet 211®, and Cinnamon oil.

Another embodiment comprises cyclosporin A, Hetsorb S-20®, and Cinnamon oil.

Another embodiment comprises cyclosporin A, Imwitor 191®, and Cinnamon oil.

Another embodiment comprises cyclosporin A, Imwitor 370®, and Cinnamon oil.

Another embodiment comprises cyclosporin A, Imwitor 375®, and Cinnamon oil.

Another embodiment comprises cyclosporin A, Imwitor 900®, and Cinnamon oil.

Another embodiment comprises cyclosporin A, Imwitor 945®, and Cinnamon oil.

Another embodiment comprises cyclosporin A, Imwitor 2020®, and Cinnamon oil.

Another embodiment comprises cyclosporin A, Kemester 5500®, and Cinnamon oil.

Another embodiment comprises cyclosporin A, Kemester 6000®, and Cinnamon oil.

Another embodiment comprises cyclosporin A, Magrabar GMC®, and Cinnamon oil.

Another embodiment comprises cyclosporin A, Magrabar GMO-CK®, and Cinnamon oil.

Another embodiment comprises cyclosporin A, Magrabar GPC-10®, and Cinnamon oil.

Another embodiment comprises cyclosporin A, Magrabar MDG-5050®, and Cinnamon oil.

Another embodiment comprises cyclosporin A, Monalube 335®, and Cinnamon oil.

Another embodiment comprises cyclosporin A, Monoglycerides Glyceryl Monestearate Archer Daniels Midland Company®, and Cinnamon oil.

Another embodiment comprises cyclosporin A, Rita GMS®, and Cinnamon oil.

Another embodiment comprises cyclosporin A, Ritamulse SCG®, and Cinnamon oil.

Another embodiment comprises cyclosporin A, Softigen 701®, and Cinnamon oil.

Another embodiment comprises cyclosporin A, Tally 100 Plus®, and Cinnamon oil.

Another embodiment comprises cyclosporin A, a Polyethylene Glycol, and Cinnamon oil.

Another embodiment comprises cyclosporin A, Emulgade PL 68/50®, and Cinnamon oil.

Another embodiment comprises cyclosporin A, Lumulse PEG®, and Cinnamon oil.

Another embodiment comprises cyclosporin A, Rhodasurf PEG-400®, and Cinnamon oil.

Another embodiment comprises cyclosporin A, Rhodasurf PEG-600®, and Cinnamon oil.

Another embodiment comprises cyclosporin A, Witconol PEG-400®, and Cinnamon oil.

Another embodiment comprises cyclosporin A, a Polymeric Surfactant, and Cinnamon oil.

Another embodiment comprises cyclosporin A, Acritamer PNC-EG®, and Cinnamon oil.

Another embodiment comprises cyclosporin A, Ag-Rho DEP-775®, and Cinnamon oil.

Another embodiment comprises cyclosporin A, APG 325N Glycoside®, and Cinnamon oil.

Another embodiment comprises cyclosporin A, Aristoflex AVC®, and Cinnamon oil.

Another embodiment comprises cyclosporin A, Aristoflex HMB®, and Cinnamon oil.

Another embodiment comprises cyclosporin A, Burco NPS-225®, and Cinnamon oil.

Another embodiment comprises cyclosporin A, Burco NPS-816®, and Cinnamon oil.

Another embodiment comprises cyclosporin A, Chemccinate 5603®, and Cinnamon oil.

Another embodiment comprises cyclosporin A, Cosmedia Guar C-261N®, and Cinnamon oil.

Another embodiment comprises cyclosporin A, Gantrez S-95®, and Cinnamon oil.

Another embodiment comprises cyclosporin A, Glucopon 220 UP®, and Cinnamon oil.

Another embodiment comprises cyclosporin A, Glucopon 225 DK®, and Cinnamon oil.

Another embodiment comprises cyclosporin A, Glucopon 425 N®, and Cinnamon oil.

Another embodiment comprises cyclosporin A, Glucopon 600 UPF, and Cinnamon oil.

Another embodiment comprises cyclosporin A, Glucopon 625 UP®, and Cinnamon oil.

Another embodiment comprises cyclosporin A, Pemulen 1621®, and Cinnamon oil.

Another embodiment comprises cyclosporin A, Pemulen 1622®, and Cinnamon oil.

Another embodiment comprises cyclosporin A, Pemulen TR-1®, and Cinnamon oil.

Another embodiment comprises cyclosporin A, Pemulen TR-2®, and Cinnamon oil.

Another embodiment comprises cyclosporin A, Plantacare 818®, and Cinnamon oil.

Another embodiment comprises cyclosporin A, Plantapon LGC Sorb®, and Cinnamon oil.

Another embodiment comprises cyclosporin A, Plantaren 1200N®, and Cinnamon oil.

Another embodiment comprises cyclosporin A, Plantaren 2000N®, and Cinnamon oil.

Another embodiment comprises cyclosporin A, Viscolam AT 64®, and Cinnamon oil.

Another embodiment comprises cyclosporin A, Viscolam AT 64P®, and Cinnamon oil.,

Another embodiment comprises cyclosporin A, Viscolam AT 100®, and Cinnamon oil.

Another embodiment comprises cyclosporin A, Viscolam MAC 7®, and Cinnamon oil.

Another embodiment comprises cyclosporin A, Viscolam SMC 20®, and Cinnamon oil.

Another embodiment comprises cyclosporin A, Witbreak RTC-323®, and Cinnamon oil.

Another embodiment comprises cyclosporin A, WSI 3700®, and Cinnamon oil.

Another embodiment comprises cyclosporin A, a Propoxylated or Ethoxylated Fatty Acid, Alcohol, or Alkyl Phenol, and Cinnamon oil.

Another embodiment comprises cyclosporin A, Antarox AA-60®, and Cinnamon oil.

Another embodiment comprises cyclosporin A, Antarox LF-224®, and Cinnamon oil.

Another embodiment comprises cyclosporin A, Burcomul DFE-45®, and Cinnamon oil.

Another embodiment comprises cyclosporin A, Burcoterge LFE-1000®, and Cinnamon oil.

Another embodiment comprises cyclosporin A, Chemal LF-25B®, and Cinnamon oil.

Another embodiment comprises cyclosporin A, Chemal LF-40B®, and Cinnamon oil.

Another embodiment comprises cyclosporin A, Dehypon LS-36®, and Cinnamon oil.

Another embodiment comprises cyclosporin A, Dehypon LS-36®, and Cinnamon oil.

Another embodiment comprises cyclosporin A, Dehypon LS-54®, and Cinnamon oil.

Another embodiment comprises cyclosporin A, Dehypon LS-54®, and Cinnamon oil.

Another embodiment comprises cyclosporin A, Delonic 100 VLF®, and Cinnamon oil.

Another embodiment comprises cyclosporin A, Delonic LF-60 MOD®, and Cinnamon oil.

Another embodiment comprises cyclosporin A, Empiderm B®, and Cinnamon oil.

Another embodiment comprises cyclosporin A, Ethylan 1206®, and Cinnamon oil.

Another embodiment comprises cyclosporin A, Ethylan NS-500K®, and Cinnamon oil.

Another embodiment comprises cyclosporin A, Ethylan NS-500LQ®, and Cinnamon oil.

Another embodiment comprises cyclosporin A, Genapol 1392®, and Cinnamon oil.

Another embodiment comprises cyclosporin A, Genapol 2317®, and Cinnamon oil.

Another embodiment comprises cyclosporin A, Genapol 26EP710®, and Cinnamon oil.

Another embodiment comprises cyclosporin A, Genapol EP 1022®, and Cinnamon oil.

Another embodiment comprises cyclosporin A, Genapol EP 1024®, and Cinnamon oil.

Another embodiment comprises cyclosporin A, Genapol EP 6068®, and Cinnamon oil.

Another embodiment comprises cyclosporin A, Genapol NP915®, and Cinnamon oil.

Another embodiment comprises cyclosporin A, Kieralon MFB®, and Cinnamon oil.

Another embodiment comprises cyclosporin A, Lumisolve CSA-80 V®, and Cinnamon oil.

Another embodiment comprises cyclosporin A, Marlowet 5001®, and Cinnamon oil.

Another embodiment comprises cyclosporin A, Marlox FK 64®, and Cinnamon oil.

Another embodiment comprises cyclosporin A, Marlox MO 124®, and Cinnamon oil.

Another embodiment comprises cyclosporin A, Marlox S 58®, and Cinnamon oil.

Another embodiment comprises cyclosporin A, Nonatell 1003®, and Cinnamon oil.

Another embodiment comprises cyclosporin A, Nonatell 1038®, and Cinnamon oil.

Another embodiment comprises cyclosporin A, Nonatell 1052®, and Cinnamon oil.

Another embodiment comprises cyclosporin A, Nonatell 1061®, and Cinnamon oil.

Another embodiment comprises cyclosporin A, Nonatell 1075®, and Cinnamon oil.

Another embodiment comprises cyclosporin A, Nonatell 1088®, and Cinnamon oil.

Another embodiment comprises cyclosporin A, Nonatell 1123®, and Cinnamon oil.

Another embodiment comprises cyclosporin A, Nonatell 1153®, and Cinnamon oil.

Another embodiment comprises cyclosporin A, Nonatell 1161®, and Cinnamon oil.

Another embodiment comprises cyclosporin A, Nonatell 1172®, and Cinnamon oil.

Another embodiment comprises cyclosporin A, Nonatell 1181®, and Cinnamon oil.

Another embodiment comprises cyclosporin A, Norfox 36®, and Cinnamon oil.

Another embodiment comprises cyclosporin A, Procetyl AWS®, and Cinnamon oil.

Another embodiment comprises cyclosporin A, Sandoxylate SX 412®, and Cinnamon oil.

Another embodiment comprises cyclosporin A, Sandoxylate SX 418®, and Cinnamon oil.

Another embodiment comprises cyclosporin A, Surfonic JL-80X®, and Cinnamon oil.

Another embodiment comprises cyclosporin A, Surfonic JL-80X-B1®, and Cinnamon oil.

Another embodiment comprises cyclosporin A, Surfonic L4-29X®, and Cinnamon oil.

Another embodiment comprises cyclosporin A, Sunonic LF®, and Cinnamon oil.

Another embodiment comprises cyclosporin A, T-Det A826®, and Cinnamon oil.

Another embodiment comprises cyclosporin A, T-Det LF-416®, and Cinnamon oil.

Another embodiment comprises cyclosporin A, Tergitol Min-Foam 1X®, and Cinnamon oil.

Another embodiment comprises cyclosporin A, Tergitol Min-Foam 2X®, and Cinnamon oil.

Another embodiment comprises cyclosporin A, Triton CF-21®, and Cinnamon oil.

Another embodiment comprises cyclosporin A, Triton CF-76®, and Cinnamon oil.

Another embodiment comprises cyclosporin A, Triton XL-80N®, and Cinnamon oil.

Another embodiment comprises cyclosporin A, Witconol NS-98®, and Cinnamon oil.

Another embodiment comprises cyclosporin A, Witconol NS-108LQ®, and Cinnamon oil.

Another embodiment comprises cyclosporin A, Witconol NS-145®, and Cinnamon oil.

Another embodiment comprises cyclosporin A, Witconol NS-179®, and Cinnamon oil.

Another embodiment comprises cyclosporin A, a Protein-based Surfactant, and Cinnamon oil.

Another embodiment comprises cyclosporin A, AminoFoam W®, and Cinnamon oil.

Another embodiment comprises cyclosporin A, Amiter LGOD-2®, and Cinnamon oil.

Another embodiment comprises cyclosporin A, Amiter LGS-2®, and Cinnamon oil.

Another embodiment comprises cyclosporin A, Amiter LGS-5®, and Cinnamon oil.

Another embodiment comprises cyclosporin A, Lamepon S®, and Cinnamon oil.

Another embodiment comprises cyclosporin A, Lamepon S®, and Cinnamon oil.

Another embodiment comprises cyclosporin A, Maypon 4C®, and Cinnamon oil.

Another embodiment comprises cyclosporin A, May-Tein C®, and Cinnamon oil.

Another embodiment comprises cyclosporin A, May-Tein CT®, and Cinnamon oil.

Another embodiment comprises cyclosporin A, May-Tein KTS®, and Cinnamon oil.

Another embodiment comprises cyclosporin A, May-Tein SY®, and Cinnamon oil.

Another embodiment comprises cyclosporin A, Plantapon S®, and Cinnamon oil.

Another embodiment comprises cyclosporin A, Proteol APL®, and Cinnamon oil.

Another embodiment comprises cyclosporin A, Proteol OAT®, and Cinnamon oil.

Another embodiment comprises cyclosporin A, Pyroter CPI-40®, and Cinnamon oil.

Another embodiment comprises cyclosporin A, Pyroter GPI-25®, and Cinnamon oil.

Another embodiment comprises cyclosporin A, Supro-Tein S®, and Cinnamon oil.

Another embodiment comprises cyclosporin A, Supro-Tein V®, and Cinnamon oil.

Another embodiment comprises cyclosporin A, ® and Cinnamon oil.

Another embodiment comprises cyclosporin A, a Sarcosine Derivative®, and Cinnamon oil.

Another embodiment comprises cyclosporin A, Crodasinic LS-30®, and Cinnamon oil.

Another embodiment comprises cyclosporin A, Vanseal CS®, and Cinnamon oil.

Another embodiment comprises cyclosporin A, Vanseal LS®, and Cinnamon oil.

Another embodiment comprises cyclosporin A, Vanseal MS®, and Cinnamon oil.

Another embodiment comprises cyclosporin A, Vanseal NACS-30®, and Cinnamon oil.

Another embodiment comprises cyclosporin A, Vanseal NALS-95®, and Cinnamon oil.

Another embodiment comprises cyclosporin A, Vanseal OS®, and Cinnamon oil.

Another embodiment comprises cyclosporin A, ® and Cinnamon oil.

Another embodiment comprises cyclosporin A, a Silicone-based Surfactant®, and Cinnamon oil.

Another embodiment comprises cyclosporin A, Abil-B-9950®, and Cinnamon oil.

Another embodiment comprises cyclosporin A, Abil Care 85®, and Cinnamon oil.

Another embodiment comprises cyclosporin A, Abil EM 90®, and Cinnamon oil.

Another embodiment comprises cyclosporin A, Abil EM 97®, and Cinnamon oil.

Another embodiment comprises cyclosporin A, Abil WE-09®, and Cinnamon oil.

Another embodiment comprises cyclosporin A, Dow Corning 1248 Fluid®, and Cinnamon oil.

Another embodiment comprises cyclosporin A, Dow Corning 3225C®, and Cinnamon oil.

Another embodiment comprises cyclosporin A, Dow Corning 5200®, and Cinnamon oil.

Another embodiment comprises cyclosporin A, Dow Corning Q4-3667®, and Cinnamon oil.

Another embodiment comprises cyclosporin A, Monasil PCA®, and Cinnamon oil.

Another embodiment comprises cyclosporin A, Monasil PDM®, and Cinnamon oil.

Another embodiment comprises cyclosporin A, Monasil PLN®, and Cinnamon oil.

Another embodiment comprises cyclosporin A, Polyderm PPI-SI-WS®, and Cinnamon oil.

Another embodiment comprises cyclosporin A, Troysol 380W®, and Cinnamon oil.

Another embodiment comprises cyclosporin A, Troysol S366®, and Cinnamon oil.

Another embodiment comprises cyclosporin A, a Sorbitan Derivative, and Cinnamon oil.

Another embodiment comprises cyclosporin A, Alkamuls SML®, and Cinnamon oil.

Another embodiment comprises cyclosporin A, Alkamuls SMO®, and Cinnamon oil.

Another embodiment comprises cyclosporin A, Alkamuls STO®, and Cinnamon oil.

Another embodiment comprises cyclosporin A, Arlacel 20®, and Cinnamon oil.

Another embodiment comprises cyclosporin A, Arlacel 40®, and Cinnamon oil.

Another embodiment comprises cyclosporin A, Arlacel 60®, and Cinnamon oil.

Another embodiment comprises cyclosporin A, Arlacel 80®, and Cinnamon oil.

Another embodiment comprises cyclosporin A, Arlacel C®, and Cinnamon oil.

Another embodiment comprises cyclosporin A, Armul 21®, and Cinnamon oil.

Another embodiment comprises cyclosporin A, Atlox 80®, and Cinnamon oil.

Another embodiment comprises cyclosporin A, Atlox 847®, and Cinnamon oil.

Another embodiment comprises cyclosporin A, Atlox 1045A®, and Cinnamon oil.

Another embodiment comprises cyclosporin A, Canarcel 20®, and Cinnamon oil.

Another embodiment comprises cyclosporin A, Canarcel 60®, and Cinnamon oil.

Another embodiment comprises cyclosporin A, Canarcel 80®, and Cinnamon oil.

Another embodiment comprises cyclosporin A, Canarcel TW 20®, and Cinnamon oil.

Another embodiment comprises cyclosporin A, Canarcel TW 60®, and Cinnamon oil.

Another embodiment comprises cyclosporin A, Canarcel TW 80®, and Cinnamon oil.

Another embodiment comprises cyclosporin A, Coladet BSB-P®, and Cinnamon oil.

Another embodiment comprises cyclosporin A, Customulse O-20®, and Cinnamon oil.

Another embodiment comprises cyclosporin A, Dehymuls E®, and Cinnamon oil.

Another embodiment comprises cyclosporin A, DeSotan SMO®, and Cinnamon oil.

Another embodiment comprises cyclosporin A, DeSotan SMO-20®, and Cinnamon oil.

Another embodiment comprises cyclosporin A, DeSotan SMT®, and Cinnamon oil.

Another embodiment comprises cyclosporin A, DeSotan SMT-20®, and Cinnamon oil.

Another embodiment comprises cyclosporin A, Durfax 60®, and Cinnamon oil.

Another embodiment comprises cyclosporin A, Durfax 65®, and Cinnamon oil.

Another embodiment comprises cyclosporin A, Durfax 80®, and Cinnamon oil.

Another embodiment comprises cyclosporin A, Durtan 60®, and Cinnamon oil.

Another embodiment comprises cyclosporin A, Durtan 65®, and Cinnamon oil.

Another embodiment comprises cyclosporin A, Liposorb L®, and Cinnamon oil.

Another embodiment comprises cyclosporin A, Liposorb L-10®, and Cinnamon oil.

Another embodiment comprises cyclosporin A, Liposorb L-20®, and Cinnamon oil.

Another embodiment comprises cyclosporin A, Liposorb O®, and Cinnamon oil.

Another embodiment comprises cyclosporin A, Liposorb O-20®, and Cinnamon oil.

Another embodiment comprises cyclosporin A, Liposorb P®, and Cinnamon oil.

Another embodiment comprises cyclosporin A, Liposorb P-20®, and Cinnamon oil.

Another embodiment comprises cyclosporin A, Liposorb S®, and Cinnamon oil.

Another embodiment comprises cyclosporin A, Liposorb S-20®, and Cinnamon oil.

Another embodiment comprises cyclosporin A, Liposorb SQO®, and Cinnamon oil.

Another embodiment comprises cyclosporin A, Liposorb TO®, and Cinnamon oil.

Another embodiment comprises cyclosporin A, Liposorb TS®, and Cinnamon oil.

Another embodiment comprises cyclosporin A, Liposorb TS-20®, and Cinnamon oil.

Another embodiment comprises cyclosporin A, Lumisorb PS®, and Cinnamon oil.

Another embodiment comprises cyclosporin A, Lumisorb SMO (T)®, and Cinnamon oil.

Another embodiment comprises cyclosporin A, Lumisorb SMS K®, and Cinnamon oil.

Another embodiment comprises cyclosporin A, Lumisorb SSO®, and Cinnamon oil.

Another embodiment comprises cyclosporin A, Lumisorb STS K®, and Cinnamon oil.

Another embodiment comprises cyclosporin A, Lumisorb STT®, and Cinnamon oil.

Another embodiment comprises cyclosporin A, Magrabar SMO®, and Cinnamon oil.

Another embodiment comprises cyclosporin A, Magrabar SMO-VEG®, and Cinnamon oil.

Another embodiment comprises cyclosporin A, Magrabar SMT®, and Cinnamon oil.

Another embodiment comprises cyclosporin A, Magrabar STO®, and Cinnamon oil.

Another embodiment comprises cyclosporin A, Miracare BC-27®, and Cinnamon oil.

Another embodiment comprises cyclosporin A, Ritabate 20®, and Cinnamon oil.

Another embodiment comprises cyclosporin A, Ritabate 40®, and Cinnamon oil.

Another embodiment comprises cyclosporin A, Ritabate 60®, and Cinnamon oil.

Another embodiment comprises cyclosporin A, Ritabate 80®, and Cinnamon oil.

Another embodiment comprises cyclosporin A, T-Maz®, and Cinnamon oil.

Another embodiment comprises cyclosporin A, Tego SML®, and Cinnamon oil.

Another embodiment comprises cyclosporin A, Tego SML 20®, and Cinnamon oil.

Another embodiment comprises cyclosporin A, Tego SMO 80 V®, and Cinnamon oil.

Another embodiment comprises cyclosporin A, Tego SMO V®, and Cinnamon oil.

Another embodiment comprises cyclosporin A, Tego SMS®, and Cinnamon oil.

Another embodiment comprises cyclosporin A, Tego STO V®, and Cinnamon oil.

Another embodiment comprises cyclosporin A, Tween 21®, and Cinnamon oil.

Another embodiment comprises cyclosporin A, Tween 40®, and Cinnamon oil.

Another embodiment comprises cyclosporin A, Tween 60®, and Cinnamon oil.

Another embodiment comprises cyclosporin A, Tween 60 K®, and Cinnamon oil.

Another embodiment comprises cyclosporin A, Tween 61®, and Cinnamon oil.

Another embodiment comprises cyclosporin A, Tween 65®, and Cinnamon oil.

Another embodiment comprises cyclosporin A, Tween 80®, and Cinnamon oil.

Another embodiment comprises cyclosporin A, Tween 80 K®, and Cinnamon oil.

Another embodiment comprises cyclosporin A, Tween 81®, and Cinnamon oil.

Another embodiment comprises cyclosporin A, Tween 85®, and Cinnamon oil.

Another embodiment comprises cyclosporin A, a Sucrose or Glucose Ester, or Derivative thereof, and Cinnamon oil.

Another embodiment comprises cyclosporin A, DeSulf GOS-P-60WCG®, and Cinnamon oil.

Another embodiment comprises cyclosporin A, Glucam E-20 Distearate®, and Cinnamon oil.

Another embodiment comprises cyclosporin A, Glucamate DOE-120®, and Cinnamon oil.

Another embodiment comprises cyclosporin A, Glucamate SSE-20®, and Cinnamon oil.

Another embodiment comprises cyclosporin A, Glucate DO®, and Cinnamon oil.

Another embodiment comprises cyclosporin A, Glucate SS®, and Cinnamon oil.

Another embodiment comprises cyclosporin A, Glucopon 425 UP®, and Cinnamon oil.

Another embodiment comprises cyclosporin A, Isolan IS®, and Cinnamon oil.

Another embodiment comprises cyclosporin A, Mazon 40®, and Cinnamon oil.

Another embodiment comprises cyclosporin A, Montanov 82®, and Cinnamon oil.

Another embodiment comprises cyclosporin A, Montanov 202®, and Cinnamon oil.

Another embodiment comprises cyclosporin A, Montanov S®, and Cinnamon oil.

Another embodiment comprises cyclosporin A, Rheozan®, and Cinnamon oil.

Another embodiment comprises cyclosporin A, Simulsol AS 48®, and Cinnamon oil.

Another embodiment comprises cyclosporin A, Simulsol SL 4®, and Cinnamon oil.

Another embodiment comprises cyclosporin A, Simulsol SL 10®, and Cinnamon oil.

Another embodiment comprises cyclosporin A, Simulsol SL 11W®, and Cinnamon oil.

Another embodiment comprises cyclosporin A, Simulsol SL 55®, and Cinnamon oil.

Another embodiment comprises cyclosporin A, Suga Nate 100 and 160®, and Cinnamon oil.

Another embodiment comprises cyclosporin A, Tego Care 450®, and Cinnamon oil.

Another embodiment comprises cyclosporin A, Tego Care CG 90®, and Cinnamon oil.

Another embodiment comprises cyclosporin A, Tego Care PS®, and Cinnamon oil.

Another embodiment comprises cyclosporin A, Tegosoft PSE 141 G®, and Cinnamon oil.

Another embodiment comprises cyclosporin A, Tegotens G 826®, and Cinnamon oil.

Another embodiment comprises cyclosporin A, Triton BG-10 (70%)®, and Cinnamon oil.

Another embodiment comprises cyclosporin A, Triton CG-110 (60%)®, and Cinnamon oil.

Another embodiment comprises cyclosporin A, Wickenol 545®, and Cinnamon oil.

Another embodiment comprises cyclosporin A, an alcohol , and an oil having a Specific Gravity between 0.90 and 0.95.

Another embodiment comprises cyclosporin A, Diglycerols, and an oil having a Specific Gravity between 0.90 and 0.95.

Another embodiment comprises cyclosporin A, Hetoxide GT-80®, and an oil having a Specific Gravity between 0.90 and 0.95.

Another embodiment comprises cyclosporin A, Lexemul BEO®, and an oil having a Specific Gravity between 0.90 and 0.95.

Another embodiment comprises cyclosporin A, Polyglycerol-3®, and an oil having a Specific Gravity between 0.90 and 0.95.

Another embodiment comprises cyclosporin A, Redicote E Series®, and an oil having a Specific Gravity between 0.90 and 0.95.

Another embodiment comprises cyclosporin A, Simulsol OX 1005L®, and an oil having a Specific Gravity between 0.90 and 0.95.

Another embodiment comprises cyclosporin A, Stanfax 567®, and an oil having a Specific Gravity between 0.90 and 0.95.

Another embodiment comprises cyclosporin A, TA-1618®, and an oil having a Specific Gravity between 0.90 and 0.95.

Another embodiment comprises cyclosporin A, Witconol H-31A®, and an oil having a Specific Gravity between 0.90 and 0.95.

Another embodiment comprises cyclosporin A, Standard Div.®, and an oil having a Specific Gravity between 0.90 and 0.95.

Another embodiment comprises cyclosporin A, an amine oxide, and an oil having a Specific Gravity between 0.90 and 0.95.

Another embodiment comprises cyclosporin A, AO-405®, and an oil having a Specific Gravity between 0.90 and 0.95.

Another embodiment comprises cyclosporin A, AO-455®, and an oil having a Specific Gravity between 0.90 and 0.95.

Another embodiment comprises cyclosporin A, AO 728 Special®, and an oil having a Specific Gravity between 0.90 and 0.95.

Another embodiment comprises cyclosporin A, Barlox 12®, and an oil having a Specific Gravity between 0.90 and 0.95.

Another embodiment comprises cyclosporin A, Barlox 14®, and an oil having a Specific Gravity between 0.90 and 0.95.

Another embodiment comprises cyclosporin A, Burcoxide Lo®, and an oil having a Specific Gravity between 0.90 and 0.95.

Another embodiment comprises cyclosporin A, Caloxamine LO®, and an oil having a Specific Gravity between 0.90 and 0.95.

Another embodiment comprises cyclosporin A, Chemoxide CAW®, and an oil having a Specific Gravity between 0.90 and 0.95.

Another embodiment comprises cyclosporin A, Chemoxide LM-30®, and an oil having a Specific Gravity between 0.90 and 0.95.

Another embodiment comprises cyclosporin A, Chemoxide LO®, and an oil having a Specific Gravity between 0.90 and 0.95.

Another embodiment comprises cyclosporin A, Chemoxide MO®, and an oil having a Specific Gravity between 0.90 and 0.95.

Another embodiment comprises cyclosporin A, Colalux CAO-35®, and an oil having a Specific Gravity between 0.90 and 0.95.

Another embodiment comprises cyclosporin A, Colalux LO®, and an oil having a Specific Gravity between 0.90 and 0.95.

Another embodiment comprises cyclosporin A, DeMox CAPO®, and an oil having a Specific Gravity between 0.90 and 0.95.

Another embodiment comprises cyclosporin A, DeMox CSG-30®, and an oil having a Specific Gravity between 0.90 and 0.95.

Another embodiment comprises cyclosporin A, DeMox LAO®, and an oil having a Specific Gravity between 0.90 and 0.95.

Another embodiment comprises cyclosporin A, Emcol L®, and an oil having a Specific Gravity between 0.90 and 0.95.

Another embodiment comprises cyclosporin A, Empigen OB®, and an oil having a Specific Gravity between 0.90 and 0.95.

Another embodiment comprises cyclosporin A, Empigen OS/A®, and an oil having a Specific Gravity between 0.90 and 0.95.

Another embodiment comprises cyclosporin A, Foamox CDO®, and an oil having a Specific Gravity between 0.90 and 0.95.

Another embodiment comprises cyclosporin A, Foamox DMM®, and an oil having a Specific Gravity between 0.90 and 0.95.

Another embodiment comprises cyclosporin A, Foamox DMS®, and an oil having a Specific Gravity between 0.90 and 0.95.

Another embodiment comprises cyclosporin A, Genaminox KC®, and an oil having a Specific Gravity between 0.90 and 0.95.

Another embodiment comprises cyclosporin A, Genaminox LA®, and an oil having a Specific Gravity between 0.90 and 0.95.

Another embodiment comprises cyclosporin A, Hartofoam SAO®, and an oil having a Specific Gravity between 0.90 and 0.95.

Another embodiment comprises cyclosporin A, Hartox DMCD®, and an oil having a Specific Gravity between 0.90 and 0.95.

Another embodiment comprises cyclosporin A, Lipowax DAT®, and an oil having a Specific Gravity between 0.90 and 0.95.

Another embodiment comprises cyclosporin A, Lipowax PB Pastilles®, and an oil having a Specific Gravity between 0.90 and 0.95.

Another embodiment comprises cyclosporin A, Mackamine C8®, and an oil having a Specific Gravity between 0.90 and 0.95.

Another embodiment comprises cyclosporin A, Mackamine C10®, and an oil having a Specific Gravity between 0.90 and 0.95.

Another embodiment comprises cyclosporin A, Mackamine C14®, and an oil having a Specific Gravity between 0.90 and 0.95.

Another embodiment comprises cyclosporin A, Mackamine CAO®, and an oil having a Specific Gravity between 0.90 and 0.95.

Another embodiment comprises cyclosporin A, Mackamine CO®, and an oil having a Specific Gravity between 0.90 and 0.95.

Another embodiment comprises cyclosporin A, Mackamine LO®, and an oil having a Specific Gravity between 0.90 and 0.95.

Another embodiment comprises cyclosporin A, Mackamine O2®, and an oil having a Specific Gravity between 0.90 and 0.95.

Another embodiment comprises cyclosporin A, Mackamine SAO®, and an oil having a Specific Gravity between 0.90 and 0.95.

Another embodiment comprises cyclosporin A, Mackamine SO®, and an oil having a Specific Gravity between 0.90 and 0.95.

Another embodiment comprises cyclosporin A, Mazox KCAO®, and an oil having a Specific Gravity between 0.90 and 0.95.

Another embodiment comprises cyclosporin A, Monalac MO®, and an oil having a Specific Gravity between 0.90 and 0.95.

Another embodiment comprises cyclosporin A, Norfox LDA®, and an oil having a Specific Gravity between 0.90 and 0.95.

Another embodiment comprises cyclosporin A, Rhodamox LO®, and an oil having a Specific Gravity between 0.90 and 0.95.

Another embodiment comprises cyclosporin A, Schercamox C-AA®, and an oil having a Specific Gravity between 0.90 and 0.95.

Another embodiment comprises cyclosporin A, Schercamox DMA®, and an oil having a Specific Gravity between 0.90 and 0.95.

Another embodiment comprises cyclosporin A, Schercamox DML®, and an oil having a Specific Gravity between 0.90 and 0.95.

Another embodiment comprises cyclosporin A, Schercamox DMM®, and an oil having a Specific Gravity between 0.90 and 0.95.

Another embodiment comprises cyclosporin A, Schercamox DMS®, and an oil having a Specific Gravity between 0.90 and 0.95.

Another embodiment comprises cyclosporin A, Tegotens DO®, and an oil having a Specific Gravity between 0.90 and 0.95.

Another embodiment comprises cyclosporin A, Tomah AO-14-2®, and an oil having a Specific Gravity between 0.90 and 0.95.

Another embodiment comprises cyclosporin A, Triaminox CDO®, and an oil having a Specific Gravity between 0.90 and 0.95.

Another embodiment comprises cyclosporin A, a block polymer, and an oil having a Specific Gravity between 0.90 and 0.95.

Another embodiment comprises cyclosporin A, AL 2070®, and an oil having a Specific Gravity between 0.90 and 0.95.

Another embodiment comprises cyclosporin A, Antarox 17-R-2®, and an oil having a Specific Gravity between 0.90 and 0.95.

Another embodiment comprises cyclosporin A, Antarox 25-R-2®, and an oil having a Specific Gravity between 0.90 and 0.95.

Another embodiment comprises cyclosporin A, Antarox 31-R-1®, and an oil having a Specific Gravity between 0.90 and 0.95.

Another embodiment comprises cyclosporin A, Antarox P-84®, and an oil having a Specific Gravity between 0.90 and 0.95.

Another embodiment comprises cyclosporin A, Antarox P-104/H®, and an oil having a Specific Gravity between 0.90 and 0.95.

Another embodiment comprises cyclosporin A, Arnox BP-Series®, and an oil having a Specific Gravity between 0.90 and 0.95.

Another embodiment comprises cyclosporin A, Chemonic 435®, and an oil having a Specific Gravity between 0.90 and 0.95.

Another embodiment comprises cyclosporin A, Chemonic D-25®, and an oil having a Specific Gravity between 0.90 and 0.95.

Another embodiment comprises cyclosporin A, Chemonic PL Series®, and an oil having a Specific Gravity between 0.90 and 0.95.

Another embodiment comprises cyclosporin A, Ethox L-121®, and an oil having a Specific Gravity between 0.90 and 0.95.

Another embodiment comprises cyclosporin A, Ethox L-122®, and an oil having a Specific Gravity between 0.90 and 0.95.

Another embodiment comprises cyclosporin A, Genapol PF-10®, and an oil having a Specific Gravity between 0.90 and 0.95.

Another embodiment comprises cyclosporin A, Genapol PF-20®, and an oil having a Specific Gravity between 0.90 and 0.95.

Another embodiment comprises cyclosporin A, Genapol PF-40A®, and an oil having a Specific Gravity between 0.90 and 0.95.

Another embodiment comprises cyclosporin A, Norfox 2-LF®, and an oil having a Specific Gravity between 0.90 and 0.95.

Another embodiment comprises cyclosporin A, Simulsol NW 342®, and an oil having a Specific Gravity between 0.90 and 0.95.

Another embodiment comprises cyclosporin A, T-Det BP-1®, and an oil having a Specific Gravity between 0.90 and 0.95.

Another embodiment comprises cyclosporin A, T-Det XD®, and an oil having a Specific Gravity between 0.90 and 0.95.

Another embodiment comprises cyclosporin A, T-Det XH®, and an oil having a Specific Gravity between 0.90 and 0.95.

Another embodiment comprises cyclosporin A, Triton CF-32®, and an oil having a Specific Gravity between 0.90 and 0.95.

Another embodiment comprises cyclosporin A, Witconol 171®, and an oil having a Specific Gravity between 0.90 and 0.95.

Another embodiment comprises cyclosporin A, Witconol 324®, and an oil having a Specific Gravity between 0.90 and 0.95.

Another embodiment comprises cyclosporin A, Witconol 324D®, and an oil having a Specific Gravity between 0.90 and 0.95.

Another embodiment comprises cyclosporin A, Witconol PD-2000®, and an oil having a Specific Gravity between 0.90 and 0.95.

Another embodiment comprises cyclosporin A, a Carboxylated Alcohol or Alkylphenol Ethoxylate, and an oil having a Specific Gravity between 0.90 and 0.95.

Another embodiment comprises cyclosporin A, Emcol CN-6®, and an oil having a Specific Gravity between 0.90 and 0.95.

Another embodiment comprises cyclosporin A, Ethcarb®, and an oil having a Specific Gravity between 0.90 and 0.95.

Another embodiment comprises cyclosporin A, Gemtex WNT-Conc®, and an oil having a Specific Gravity between 0.90 and 0.95.

Another embodiment comprises cyclosporin A, Incrodet TD7-C®, and an oil having a Specific Gravity between 0.90 and 0.95.

Another embodiment comprises cyclosporin A, Marlinat CM 105/80®, and an oil having a Specific Gravity between 0.90 and 0.95.

Another embodiment comprises cyclosporin A, Marlowet 1072®, and an oil having a Specific Gravity between 0.90 and 0.95.

Another embodiment comprises cyclosporin A, Marlowet 4530®, and an oil having a Specific Gravity between 0.90 and 0.95.

Another embodiment comprises cyclosporin A, Marlowet 4530 LF®, and an oil having a Specific Gravity between 0.90 and 0.95.

Another embodiment comprises cyclosporin A, Marlowet 4534®, and an oil having a Specific Gravity between 0.90 and 0.95.

Another embodiment comprises cyclosporin A, Marlowet 4538®, and an oil having a Specific Gravity between 0.90 and 0.95.

Another embodiment comprises cyclosporin A, Marlowet 4539®, and an oil having a Specific Gravity between 0.90 and 0.95.

Another embodiment comprises cyclosporin A, Marlowet 4539 LF®, and an oil having a Specific Gravity between 0.90 and 0.95.

Another embodiment comprises cyclosporin A, Marlowet 4541®, and an oil having a Specific Gravity between 0.90 and 0.95.

Another embodiment comprises cyclosporin A, Miranate LEC-80®, and an oil having a Specific Gravity between 0.90 and 0.95.

Another embodiment comprises cyclosporin A, Sandopan B®, and an oil having a Specific Gravity between 0.90 and 0.95.

Another embodiment comprises cyclosporin A, Sandopan B Modified®, and an oil having a Specific Gravity between 0.90 and 0.95.

Another embodiment comprises cyclosporin A, Sandopan LS-24 Gel®, and an oil having a Specific Gravity between 0.90 and 0.95.

Another embodiment comprises cyclosporin A, Surfine T-A®, and an oil having a Specific Gravity between 0.90 and 0.95.

Another embodiment comprises cyclosporin A, a Carboxylic Acid/Fatty Acid , and an oil having a Specific Gravity between 0.90 and 0.95.

Another embodiment comprises cyclosporin A, Colaterge RAM®, and an oil having a Specific Gravity between 0.90 and 0.95.

Another embodiment comprises cyclosporin A, Colatrope INC®, and an oil having a Specific Gravity between 0.90 and 0.95.

Another embodiment comprises cyclosporin A, Crodacid B®, and an oil having a Specific Gravity between 0.90 and 0.95.

Another embodiment comprises cyclosporin A, DeTrope CA-100®, and an oil having a Specific Gravity between 0.90 and 0.95.

Another embodiment comprises cyclosporin A, Latol MTO®, and an oil having a Specific Gravity between 0.90 and 0.95.

Another embodiment comprises cyclosporin A, Lumulse CC-33 K®, and an oil having a Specific Gravity between 0.90 and 0.95.

Another embodiment comprises cyclosporin A, Mulls 2218®, and an oil having a Specific Gravity between 0.90 and 0.95.

Another embodiment comprises cyclosporin A, OL-600®, and an oil having a Specific Gravity between 0.90 and 0.95.

Another embodiment comprises cyclosporin A, OL-800®, and an oil having a Specific Gravity between 0.90 and 0.95.

Another embodiment comprises cyclosporin A, R-910®, and an oil having a Specific Gravity between 0.90 and 0.95.

Another embodiment comprises cyclosporin A, S-210®, and an oil having a Specific Gravity between 0.90 and 0.95.

Another embodiment comprises cyclosporin A, Sandopan DTC Acid®, and an oil having a Specific Gravity between 0.90 and 0.95.

Another embodiment comprises cyclosporin A, Sandopan LS 24 N®, and an oil having a Specific Gravity between 0.90 and 0.95.

Another embodiment comprises cyclosporin A, Sandopan MA-18®, and an oil having a Specific Gravity between 0.90 and 0.95.

Another embodiment comprises cyclosporin A, an Ethoxylated Alcohol , and an oil having a Specific Gravity between 0.90 and 0.95.

Another embodiment comprises cyclosporin A, Adsee 799®, and an oil having a Specific Gravity between 0.90 and 0.95.

Another embodiment comprises cyclosporin A, Adsee 799®, and an oil having a Specific Gravity between 0.90 and 0.95.

Another embodiment comprises cyclosporin A, Alfonic 610-3.5®, and an oil having a Specific Gravity between 0.90 and 0.95.

Another embodiment comprises cyclosporin A, Alfonic 810-2®, and an oil having a Specific Gravity between 0.90 and 0.95.

Another embodiment comprises cyclosporin A, Alfonic 810-6®, and an oil having a Specific Gravity between 0.90 and 0.95.

Another embodiment comprises cyclosporin A, Alfonic 1012-3®, and an oil having a Specific Gravity between 0.90 and 0.95.

Another embodiment comprises cyclosporin A, Alfonic 1012-5®, and an oil having a Specific Gravity between 0.90 and 0.95.

Another embodiment comprises cyclosporin A, Alfonic 1216CO-1.5®, and an oil having a Specific Gravity between 0.90 and 0.95.

Another embodiment comprises cyclosporin A, Alfonic 1216CO-7®, and an oil having a Specific Gravity between 0.90 and 0.95.

Another embodiment comprises cyclosporin A, Alfonic 1412-3®, and an oil having a Specific Gravity between 0.90 and 0.95.

Another embodiment comprises cyclosporin A, Alfonic 1412-7®, and an oil having a Specific Gravity between 0.90 and 0.95.

Another embodiment comprises cyclosporin A, Arlasolve 200®, and an oil having a Specific Gravity between 0.90 and 0.95.

Another embodiment comprises cyclosporin A, Arlasolve 200 Liquid®, and an oil having a Specific Gravity between 0.90 and 0.95.

Another embodiment comprises cyclosporin A, Armix 180-C®, and an oil having a Specific Gravity between 0.90 and 0.95.

Another embodiment comprises cyclosporin A, Armix 183®, and an oil having a Specific Gravity between 0.90 and 0.95.

Another embodiment comprises cyclosporin A, Armul 2404®, and an oil having a Specific Gravity between 0.90 and 0.95.

Another embodiment comprises cyclosporin A, Armul 2404®, and an oil having a Specific Gravity between 0.90 and 0.95.

Another embodiment comprises cyclosporin A, Atlas EMJ-C®, and an oil having a Specific Gravity between 0.90 and 0.95.

Another embodiment comprises cyclosporin A, Atlas G-2109®, and an oil having a Specific Gravity between 0.90 and 0.95.

Another embodiment comprises cyclosporin A, Atlas G-3886®, and an oil having a Specific Gravity between 0.90 and 0.95.

Another embodiment comprises cyclosporin A, Atlas G-38905, and an oil having a Specific Gravity between 0.90 and 0.95.

Another embodiment comprises cyclosporin A, Bio Soft E-200®, and an oil having a Specific Gravity between 0.90 and 0.95.

Another embodiment comprises cyclosporin A, Bio Soft E-300®, and an oil having a Specific Gravity between 0.90 and 0.95.

Another embodiment comprises cyclosporin A, Bio Soft E-400®, and an oil having a Specific Gravity between 0.90 and 0.95.

Another embodiment comprises cyclosporin A, Bio Soft EN 600®, and an oil having a Specific Gravity between 0.90 and 0.95.

Another embodiment comprises cyclosporin A, Bio Soft TD-400®, and an oil having a Specific Gravity between 0.90 and 0.95.

Another embodiment comprises cyclosporin A, Bio Soft TD-63®, and an oil having a Specific Gravity between 0.90 and 0.95.

Another embodiment comprises cyclosporin A, Brij 30®, and an oil having a Specific Gravity between 0.90 and 0.95.

Another embodiment comprises cyclosporin A, Brij 52®, and an oil having a Specific Gravity between 0.90 and 0.95.

Another embodiment comprises cyclosporin A, Brij 56®, and an oil having a Specific Gravity between 0.90 and 0.95.

Another embodiment comprises cyclosporin A, Brij 58®, and an oil having a Specific Gravity between 0.90 and 0.95.

Another embodiment comprises cyclosporin A, Brij 72®, and an oil having a Specific Gravity between 0.90 and 0.95.

Another embodiment comprises cyclosporin A, Brij 76®, and an oil having a Specific Gravity between 0.90 and 0.95.

Another embodiment comprises cyclosporin A, Brij 78®, and an oil having a Specific Gravity between 0.90 and 0.95.

Another embodiment comprises cyclosporin A, Brij 93®, and an oil having a Specific Gravity between 0.90 and 0.95.

Another embodiment comprises cyclosporin A, Brij 97®, and an oil having a Specific Gravity between 0.90 and 0.95.

Another embodiment comprises cyclosporin A, Brij 98®, and an oil having a Specific Gravity between 0.90 and 0.95.

Another embodiment comprises cyclosporin A, Brij 700®, and an oil having a Specific Gravity between 0.90 and 0.95.

Another embodiment comprises cyclosporin A, Brij 700 S®, and an oil having a Specific Gravity between 0.90 and 0.95.

Another embodiment comprises cyclosporin A, Brij 721®, and an oil having a Specific Gravity between 0.90 and 0.95.

Another embodiment comprises cyclosporin A, Brij 721 S®, and an oil having a Specific Gravity between 0.90 and 0.95.

Another embodiment comprises cyclosporin A, Burcoterge CDG®, and an oil having a Specific Gravity between 0.90 and 0.95.

Another embodiment comprises cyclosporin A, Canasol AT 600®, and an oil having a Specific Gravity between 0.90 and 0.95.

Another embodiment comprises cyclosporin A, Canasol AT 800®, and an oil having a Specific Gravity between 0.90 and 0.95.

Another embodiment comprises cyclosporin A, Canasol AT 1200®, and an oil having a Specific Gravity between 0.90 and 0.95.

Another embodiment comprises cyclosporin A, Canasol BJ 35®, and an oil having a Specific Gravity between 0.90 and 0.95.

Another embodiment comprises cyclosporin A, Canasol BJ 36®, and an oil having a Specific Gravity between 0.90 and 0.95.

Another embodiment comprises cyclosporin A, Canasol BJ 52®, and an oil having a Specific Gravity between 0.90 and 0.95.

Another embodiment comprises cyclosporin A, Canasol BJ 58®, and an oil having a Specific Gravity between 0.90 and 0.95.

Another embodiment comprises cyclosporin A, Canasol BJ 72®, and an oil having a Specific Gravity between 0.90 and 0.95.

Another embodiment comprises cyclosporin A, Canasol BJ 78®, and an oil having a Specific Gravity between 0.90 and 0.95.

Another embodiment comprises cyclosporin A, Canasol BJ 98®, and an oil having a Specific Gravity between 0.90 and 0.95.

Another embodiment comprises cyclosporin A, Canasol BJ 307®, and an oil having a Specific Gravity between 0.90 and 0.95.

Another embodiment comprises cyclosporin A, Cerfak 1400®, and an oil having a Specific Gravity between 0.90 and 0.95.

Another embodiment comprises cyclosporin A, Cetomacrogol 1000 BP®, and an oil having a Specific Gravity between 0.90 and 0.95.

Another embodiment comprises cyclosporin A, Chemonic C-2®, and an oil having a Specific Gravity between 0.90 and 0.95.

Another embodiment comprises cyclosporin A, Chemonic C-10®, and an oil having a Specific Gravity between 0.90 and 0.95.

Another embodiment comprises cyclosporin A, Chemonic C-20®, and an oil having a Specific Gravity between 0.90 and 0.95.

Another embodiment comprises cyclosporin A, Chemonic CT-12®, and an oil having a Specific Gravity between 0.90 and 0.95.

Another embodiment comprises cyclosporin A, Chemonic CT-20®, and an oil having a Specific Gravity between 0.90 and 0.95.

Another embodiment comprises cyclosporin A, Chemonic CT-30®, and an oil having a Specific Gravity between 0.90 and 0.95.

Another embodiment comprises cyclosporin A, Chemonic CT-55®, and an oil having a Specific Gravity between 0.90 and 0.95.

Another embodiment comprises cyclosporin A, Chemonic G-7®, and an oil having a Specific Gravity between 0.90 and 0.95.

Another embodiment comprises cyclosporin A, Chemonic G-26®, and an oil having a Specific Gravity between 0.90 and 0.95.

Another embodiment comprises cyclosporin A, Chemonic L-4®, and an oil having a Specific Gravity between 0.90 and 0.95.

Another embodiment comprises cyclosporin A, Chemonic L-7®, and an oil having a Specific Gravity between 0.90 and 0.95.

Another embodiment comprises cyclosporin A, Chemonic L-12®, and an oil having a Specific Gravity between 0.90 and 0.95.

Another embodiment comprises cyclosporin A, Chemonic L-23®, and an oil having a Specific Gravity between 0.90 and 0.95.

Another embodiment comprises cyclosporin A, Chemonic O-2®, and an oil having a Specific Gravity between 0.90 and 0.95.

Another embodiment comprises cyclosporin A, Chemonic O-5®, and an oil having a Specific Gravity between 0.90 and 0.95.

Another embodiment comprises cyclosporin A, Chemonic O-10®, and an oil having a Specific Gravity between 0.90 and 0.95.

Another embodiment comprises cyclosporin A, Chemonic O-20®, and an oil having a Specific Gravity between 0.90 and 0.95.

Another embodiment comprises cyclosporin A, Chemonic S-2®, and an oil having a Specific Gravity between 0.90 and 0.95.

Another embodiment comprises cyclosporin A, Chemonic S-10®, and an oil having a Specific Gravity between 0.90 and 0.95.

Another embodiment comprises cyclosporin A, Chemonic S-20®, and an oil having a Specific Gravity between 0.90 and 0.95.

Another embodiment comprises cyclosporin A, Colamulse FE®, and an oil having a Specific Gravity between 0.90 and 0.95.

Another embodiment comprises cyclosporin A, Cremophor A 20®, and an oil having a Specific Gravity between 0.90 and 0.95.

Another embodiment comprises cyclosporin A, Cremophor SA 2®, and an oil having a Specific Gravity between 0.90 and 0.95.

Another embodiment comprises cyclosporin A, Dehydol 100®, and an oil having a Specific Gravity between 0.90 and 0.95.

Another embodiment comprises cyclosporin A, Dehydol O-4®, and an oil having a Specific Gravity between 0.90 and 0.95.

Another embodiment comprises cyclosporin A, Delonic C-18®, and an oil having a Specific Gravity between 0.90 and 20 0.95.

Another embodiment comprises cyclosporin A, DeSonic 6T®, and an oil having a Specific Gravity between 0.90 and 0.95.

Another embodiment comprises cyclosporin A, DeSonic 9D®, and an oil having a Specific Gravity between 0.90 and 0.95.

Another embodiment comprises cyclosporin A, DeSonic 9T®, and an oil having a Specific Gravity between 0.90 and 0.95.

Another embodiment comprises cyclosporin A, DeSonic 12D®, and an oil having a Specific Gravity between 0.90 and 0.95.

Another embodiment comprises cyclosporin A, DeSonic 12T®, and an oil having a Specific Gravity between 0.90 and 30 0.95.

Another embodiment comprises cyclosporin A, DeSonic 15T®, and an oil having a Specific Gravity between 0.90 and 0.95.

Another embodiment comprises cyclosporin A, DeSonic TDA-9®, and an oil having a Specific Gravity between 0.90 and 0.95.

Another embodiment comprises cyclosporin A, DeThox GLG-7®, and an oil having a Specific Gravity between 0.90 and 0.95.

Another embodiment comprises cyclosporin A, DeThox GLG-26®, and an oil having a Specific Gravity between 0.90 and 0.95.

Another embodiment comprises cyclosporin A, DeThox LA-4®, and an oil having a Specific Gravity between 0.90 and 40 0.95.

Another embodiment comprises cyclosporin A, DeThox LA-23®, and an oil having a Specific Gravity between 0.90 and 0.95.

Another embodiment comprises cyclosporin A, DeThox SA-80®, and an oil having a Specific Gravity between 0.90 and 0.95.

Another embodiment comprises cyclosporin A, Disponil O5®, and an oil having a Specific Gravity between 0.90 and 0.95.

Another embodiment comprises cyclosporin A, Eccoterge EO-41 B®, and an oil having a Specific Gravity between 0.90 and 0.95.

Another embodiment comprises cyclosporin A, Empilan KA2.5/90®, and an oil having a Specific Gravity between 0.90 and 0.95.

Another embodiment comprises cyclosporin A, Empilan KA5/90®, and an oil having a Specific Gravity between 0.90 and 0.95.

Another embodiment comprises cyclosporin A, Empilan KM-20®, and an oil having a Specific Gravity between 0.90 and 0.95.

Another embodiment comprises cyclosporin A, Empilan KM-50®, and an oil having a Specific Gravity between 0.90 and 0.95.

Another embodiment comprises cyclosporin A, Empilan L-23®, and an oil having a Specific Gravity between 0.90 and 0.95.

Another embodiment comprises cyclosporin A, Ethylan 25-3®, and an oil having a Specific Gravity between 0.90 and 0.95.

Another embodiment comprises cyclosporin A, Ethylan 1204®, and an oil having a Specific Gravity between 0.90 and 0.95.

Another embodiment comprises cyclosporin A, Ethylan DA-4®, and an oil having a Specific Gravity between 0.90 and 0.95.

Another embodiment comprises cyclosporin A, Ethylan LA-230®, and an oil having a Specific Gravity between 0.90 and 0.95.

Another embodiment comprises cyclosporin A, Ethylan SN®, and an oil having a Specific Gravity between 0.90 and 0.95.

Another embodiment comprises cyclosporin A, Ethylan TD-60®, and an oil having a Specific Gravity between 0.90 and 0.95.

Another embodiment comprises cyclosporin A, Ethylan TD-100®, and an oil having a Specific Gravity between 0.90 and 0.95.

Another embodiment comprises cyclosporin A, Ethylan TD-1407®, and an oil having a Specific Gravity between 0.90 and 0.95.

Another embodiment comprises cyclosporin A, Eumulgin B1®, and an oil having a Specific Gravity between 0.90 and 0.95.

Another embodiment comprises cyclosporin A, Eumulgin B2®, and an oil having a Specific Gravity between 0.90 and 0.95.

Another embodiment comprises cyclosporin A, Eumulgin B3®, and an oil having a Specific Gravity between 0.90 and 0.95.

Another embodiment comprises cyclosporin A, Eumulgin O-10®, and an oil having a Specific Gravity between 0.90 and 0.95.

Another embodiment comprises cyclosporin A, Flo Mo 80/20®, and an oil having a Specific Gravity between 0.90 and 0.95.

Another embodiment comprises cyclosporin A, Flo Mo Low Foam®, and an oil having a Specific Gravity between 0.90 and 0.95.

Another embodiment comprises cyclosporin A, Forlan C-24®, and an oil having a Specific Gravity between 0.90 and 0.95.

Another embodiment comprises cyclosporin A, Genapol 1454®, and an oil having a Specific Gravity between 0.90 and 0.95.

Another embodiment comprises cyclosporin A, Genapol BA-020®, and an oil having a Specific Gravity between 0.90 and 0.95.

Another embodiment comprises cyclosporin A, Genapol BA-040®, and an oil having a Specific Gravity between 0.90 and 0.95.

Another embodiment comprises cyclosporin A, Genapol C-100®, and an oil having a Specific Gravity between 0.90 and 0.95.

Another embodiment comprises cyclosporin A, Genapol DA 060®, and an oil having a Specific Gravity between 0.90 and 0.95.

Another embodiment comprises cyclosporin A, Genapol HS 020®, and an oil having a Specific Gravity between 0.90 and 0.95.

Another embodiment comprises cyclosporin A, Genapol HS 200®, and an oil having a Specific Gravity between 0.90 and 0.95.

Another embodiment comprises cyclosporin A, Genapol ID-040®, and an oil having a Specific Gravity between 0.90 and 0.95.

Another embodiment comprises cyclosporin A, Genapol ID-060®, and an oil having a Specific Gravity between 0.90 and 0.95.

Another embodiment comprises cyclosporin A, Genapol ID-090®, and an oil having a Specific Gravity between 0.90 and 0.95.

Another embodiment comprises cyclosporin A, Genapol LA 010®, and an oil having a Specific Gravity between 0.90 and 0.95.

Another embodiment comprises cyclosporin A, Genapol LA 020®, and an oil having a Specific Gravity between 0.90 and 0.95.

Another embodiment comprises cyclosporin A, Genapol LA 030®, and an oil having a Specific Gravity between 0.90 and 0.95.

Another embodiment comprises cyclosporin A, Genapol LA 040®, and an oil having a Specific Gravity between 0.90 and 0.95.

Another embodiment comprises cyclosporin A, Genapol LA 050®, and an oil having a Specific Gravity between 0.90 and 0.95.

Another embodiment comprises cyclosporin A, Genapol LA 060®, and an oil having a Specific Gravity between 0.90 and 0.95.

Another embodiment comprises cyclosporin A, Genapol LA 070®, and an oil having a Specific Gravity between 0.90 and 0.95.

Another embodiment comprises cyclosporin A, Genapol LA 070S®, and an oil having a Specific Gravity between 0.90 and 0.95.

Another embodiment comprises cyclosporin A, Genapol LA 230®, and an oil having a Specific Gravity between 0.90 and 0.95.

Another embodiment comprises cyclosporin A, Genapol O 020®, and an oil having a Specific Gravity between 0.90 and 0.95.

Another embodiment comprises cyclosporin A, Genapol O 050®, and an oil having a Specific Gravity between 0.90 and 0.95.

Another embodiment comprises cyclosporin A, Genapol O 100®, and an oil having a Specific Gravity between 0.90 and 0.95.

Another embodiment comprises cyclosporin A, Genapol O 200®, and an oil having a Specific Gravity between 0.90 and 0.95.

Another embodiment comprises cyclosporin A, Genapol SA 030®, and an oil having a Specific Gravity between 0.90 and 0.95.

Another embodiment comprises cyclosporin A, Genapol SA 120®, and an oil having a Specific Gravity between 0.90 and 0.95.

Another embodiment comprises cyclosporin A, Genapol T-020®, and an oil having a Specific Gravity between 0.90 and 0.95.

Another embodiment comprises cyclosporin A, Genapol UD-030®, and an oil having a Specific Gravity between 0.90 and 0.95.

Another embodiment comprises cyclosporin A, Genapol UD-050®, and an oil having a Specific Gravity between 0.90 and 0.95.

Another embodiment comprises cyclosporin A, Genapol UD-070®, and an oil having a Specific Gravity between 0.90 and 0.95.

Another embodiment comprises cyclosporin A, Genapol UD-079®, and an oil having a Specific Gravity between 0.90 and 0.95.

Another embodiment comprises cyclosporin A, Genapol UD-080®, and an oil having a Specific Gravity between 0.90 and 0.95.

Another embodiment comprises cyclosporin A, Genapol UD-110®, and an oil having a Specific Gravity between 0.90 and 0.95.

Another embodiment comprises cyclosporin A, Genapol X 030®, and an oil having a Specific Gravity between 0.90 and 0.95.

Another embodiment comprises cyclosporin A, Genapol X 050®, and an oil having a Specific Gravity between 0.90 and 0.95.

Another embodiment comprises cyclosporin A, Genapol X 060®, and an oil having a Specific Gravity between 0.90 and 0.95.

Another embodiment comprises cyclosporin A, Genapol X 070®, and an oil having a Specific Gravity between 0.90 and 0.95.

Another embodiment comprises cyclosporin A, Genapol X 080®, and an oil having a Specific Gravity between 0.90 and 0.95.

Another embodiment comprises cyclosporin A, Genapol X 100®, and an oil having a Specific Gravity between 0.90 and 0.95.

Another embodiment comprises cyclosporin A, Genapol X159®, and an oil having a Specific Gravity between 0.90 and 0.95.

Another embodiment comprises cyclosporin A, Generol 122 E5®, and an oil having a Specific Gravity between 0.90 and 0.95.

Another embodiment comprises cyclosporin A, Generol 122 E25®, and an oil having a Specific Gravity between 0.90 and 0.95.

Another embodiment comprises cyclosporin A, Hostacerin T-3®, and an oil having a Specific Gravity between 0.90 and 0.95.

Another embodiment comprises cyclosporin A, Iconol LF 110®, and an oil having a Specific Gravity between 0.90 and 0.95.

Another embodiment comprises cyclosporin A, Incropol CS-20®, and an oil having a Specific Gravity between 0.90 and 0.95.

Another embodiment comprises cyclosporin A, Lexemul CS-20®, and an oil having a Specific Gravity between 0.90 and 0.95.

Another embodiment comprises cyclosporin A, Liponic EG-1®, and an oil having a Specific Gravity between 0.90 and 0.95.

Another embodiment comprises cyclosporin A, Lipowax D®, and an oil having a Specific Gravity between 0.90 and 0.95.

Another embodiment comprises cyclosporin A, Lipowax G®, and an oil having a Specific Gravity between 0.90 and 0.95.

Another embodiment comprises cyclosporin A, Lipowax NI®, and an oil having a Specific Gravity between 0.90 and 0.95.

Another embodiment comprises cyclosporin A, Lipowax P®, and an oil having a Specific Gravity between 0.90 and 0.95.

Another embodiment comprises cyclosporin A, Lipowax P-31®, and an oil having a Specific Gravity between 0.90 and 0.95.

Another embodiment comprises cyclosporin A, Lipowax PR®, and an oil having a Specific Gravity between 0.90 and 0.95.

Another embodiment comprises cyclosporin A, Lumulse CS-20®, and an oil having a Specific Gravity between 0.90 and 0.95.

Another embodiment comprises cyclosporin A, Macol CSA-20®, and an oil having a Specific Gravity between 0.90 and 0.95.

Another embodiment comprises cyclosporin A, Marlox B 24/50®, and an oil having a Specific Gravity between 0.90 and 0.95.

Another embodiment comprises cyclosporin A, Mazawet 77®, and an oil having a Specific Gravity between 0.90 and 0.95.

Another embodiment comprises cyclosporin A, Norfox 1713®, and an oil having a Specific Gravity between 0.90 and 0.95.

Another embodiment comprises cyclosporin A, Norfox 2579®, and an oil having a Specific Gravity between 0.90 and 0.95.

Another embodiment comprises cyclosporin A, Norfox Lo Foam®, and an oil having a Specific Gravity between 0.90 and 0.95.

Another embodiment comprises cyclosporin A, Promulgen D®, and an oil having a Specific Gravity between 0.90 and 0.95.

Another embodiment comprises cyclosporin A, Promulgen G®, and an oil having a Specific Gravity between 0.90 and 0.95.

Another embodiment comprises cyclosporin A, Renex 30®, and an oil having a Specific Gravity between 0.90 and 0.95.

Another embodiment comprises cyclosporin A, Renex 36®, and an oil having a Specific Gravity between 0.90 and 0.95.

Another embodiment comprises cyclosporin A, Rhodasurf A 24®, and an oil having a Specific Gravity between 0.90 and 0.95.

Another embodiment comprises cyclosporin A, Rhodasurf AAE-10®, and an oil having a Specific Gravity between 0.90 and 0.95.

Another embodiment comprises cyclosporin A, Rhodasurf BEH-25®, and an oil having a Specific Gravity between 0.90 and 0.95.

Another embodiment comprises cyclosporin A, Rhodasurf BEH-40®, and an oil having a Specific Gravity between 0.90 and 0.95.

Another embodiment comprises cyclosporin A, Rhodasurf DA 530®, and an oil having a Specific Gravity between 0.90 and 0.95.

Another embodiment comprises cyclosporin A, Rhodasurf DA 630®, and an oil having a Specific Gravity between 0.90 and 0.95.

Another embodiment comprises cyclosporin A, Rhodasurf DA 639®, and an oil having a Specific Gravity between 0.90 and 0.95.

Another embodiment comprises cyclosporin A, Rhodasurf LAN-23®, and an oil having a Specific Gravity between 0.90 and 0.95.

Another embodiment comprises cyclosporin A, Rhodasurf ON-870®, and an oil having a Specific Gravity between 0.90 and 0.95.

Another embodiment comprises cyclosporin A, Rhodasurf ON-877®, and an oil having a Specific Gravity between 0.90 and 0.95.

Another embodiment comprises cyclosporin A, Rhodasurf TB-970 FLK®, and an oil having a Specific Gravity between 0.90 and 0.95.

Another embodiment comprises cyclosporin A, Ritacet-20®, and an oil having a Specific Gravity between 0.90 and 0.95.

Another embodiment comprises cyclosporin A, Ritachol 1000®, and an oil having a Specific Gravity between 0.90 and 0.95.

Another embodiment comprises cyclosporin A, Ritachol 2000®, and an oil having a Specific Gravity between 0.90 and 0.95.

Another embodiment comprises cyclosporin A, Ritachol 5000®, and an oil having a Specific Gravity between 0.90 and 0.95.

Another embodiment comprises cyclosporin A, Ritox 35®, and an oil having a Specific Gravity between 0.90 and 0.95.

Another embodiment comprises cyclosporin A, Surfonic DA-4®, and an oil having a Specific Gravity between 0.90 and 0.95.

Another embodiment comprises cyclosporin A, Surfonic DA-6®, and an oil having a Specific Gravity between 0.90 and 0.95.

Another embodiment comprises cyclosporin A, Surfonic L46-7®, and an oil having a Specific Gravity between 0.90 and 0.95.

Another embodiment comprises cyclosporin A, Surfonic POA®, and an oil having a Specific Gravity between 0.90 and 0.95.

Another embodiment comprises cyclosporin A, Synthrapol KB®, and an oil having a Specific Gravity between 0.90 and 0.95.

Another embodiment comprises cyclosporin A, Teginacid®, and an oil having a Specific Gravity between 0.90 and 0.95.

Another embodiment comprises cyclosporin A, Teginacid C®, and an oil having a Specific Gravity between 0.90 and 0.95.

Another embodiment comprises cyclosporin A, Tegotens EC 11®, and an oil having a Specific Gravity between 0.90 and 0.95.

Another embodiment comprises cyclosporin A, Tinegal NA®, and an oil having a Specific Gravity between 0.90 and 0.95.

Another embodiment comprises cyclosporin A, Tomadol 400®, and an oil having a Specific Gravity between 0.90 and 0.95.

Another embodiment comprises cyclosporin A, Tomadol 600®, and an oil having a Specific Gravity between 0.90 and 0.95.

Another embodiment comprises cyclosporin A, Tomadol 900®, and an oil having a Specific Gravity between 0.90 and 0.95.

Another embodiment comprises cyclosporin A, Uniperol O®, and an oil having a Specific Gravity between 0.90 and 0.95.

Another embodiment comprises cyclosporin A, Witconol SN Series®, and an oil having a Specific Gravity between 0.90 and 0.95.

Another embodiment comprises cyclosporin A, an Ethoxylated Alkylphenol , and an oil having a Specific Gravity between 0.90 and 0.95.

Another embodiment comprises cyclosporin A, Antarox LF-222®, and an oil having a Specific Gravity between 0.90 and 0.95.

Another embodiment comprises cyclosporin A, Atlox 775®, and an oil having a Specific Gravity between 0.90 and 0.95.

Another embodiment comprises cyclosporin A, Caloxylate N-9®, and an oil having a Specific Gravity between 0.90 and 0.95.

Another embodiment comprises cyclosporin A, Canasol NF-1000®, and an oil having a Specific Gravity between 0.90 and 0.95.

Another embodiment comprises cyclosporin A, Canasol NF-3000®, and an oil having a Specific Gravity between 0.90 and 0.95.

Another embodiment comprises cyclosporin A, Canasol NF-3070®, and an oil having a Specific Gravity between 0.90 and 0.95.

Another embodiment comprises cyclosporin A, Canasol OF 1670®, and an oil having a Specific Gravity between 0.90 and 0.95.

Another embodiment comprises cyclosporin A, Canasol OF 2570®, and an oil having a Specific Gravity between 0.90 and 0.95.

Another embodiment comprises cyclosporin A, Canasol OF 4070®, and an oil having a Specific Gravity between 0.90 and 0.95.

Another embodiment comprises cyclosporin A, Chemax DNP-8®, and an oil having a Specific Gravity between 0.90 and 0.95.

Another embodiment comprises cyclosporin A, Chemax DNP-18®, and an oil having a Specific Gravity between 0.90 and 0.95.

Another embodiment comprises cyclosporin A, Chemax DNP-150/50®, and an oil having a Specific Gravity between 0.90 and 0.95.

Another embodiment comprises cyclosporin A, DeSonic 1.5N®, and an oil having a Specific Gravity between 0.90 and 0.95.

Another embodiment comprises cyclosporin A, DeSonic 4N®, and an oil having a Specific Gravity between 0.90 and 0.95.

Another embodiment comprises cyclosporin A, DeSonic 5N®, and an oil having a Specific Gravity between 0.90 and 0.95.

Another embodiment comprises cyclosporin A, DeSonic 6D®, and an oil having a Specific Gravity between 0.90 and 0.95.

Another embodiment comprises cyclosporin A, DeSonic 6N®, and an oil having a Specific Gravity between 0.90 and 0.95.

Another embodiment comprises cyclosporin A, DeSonic 7N®, and an oil having a Specific Gravity between 0.90 and 0.95.

Another embodiment comprises cyclosporin A, DeSonic 9N®, and an oil having a Specific Gravity between 0.90 and 0.95.

Another embodiment comprises cyclosporin A, DeSonic 10D®, and an oil having a Specific Gravity between 0.90 and 0.95.

Another embodiment comprises cyclosporin A, DeSonic 11N®, and an oil having a Specific Gravity between 0.90 and 0.95.

Another embodiment comprises cyclosporin A, DeSonic 12N®, and an oil having a Specific Gravity between 0.90 and 0.95.

Another embodiment comprises cyclosporin A, DeSonic 15N®, and an oil having a Specific Gravity between 0.90 and 0.95.

Another embodiment comprises cyclosporin A, DeSonic 20N®, and an oil having a Specific Gravity between 0.90 and 0.95.

Another embodiment comprises cyclosporin A, Eccoscour RC®, and an oil having a Specific Gravity between 0.90 and 0.95.

Another embodiment comprises cyclosporin A, Eccoterge EO-100®, and an oil having a Specific Gravity between 0.90 and 0.95.

Another embodiment comprises cyclosporin A, Emulsifier 632/90%®, and an oil having a Specific Gravity between 0.90 and 0.95.

Another embodiment comprises cyclosporin A, Geronol AG-821®, and an oil having a Specific Gravity between 0.90 and 0.95.

Another embodiment comprises cyclosporin A, Gradonic N-95®, and an oil having a Specific Gravity between 0.90 and 0.95.

Another embodiment comprises cyclosporin A, Hetoxide NP-4®, and an oil having a Specific Gravity between 0.90 and 0.95.

Another embodiment comprises cyclosporin A, Hetoxide NP-30®, and an oil having a Specific Gravity between 0.90 and 0.95.

Another embodiment comprises cyclosporin A, Hostapal N-100®, and an oil having a Specific Gravity between 0.90 and 0.95.

Another embodiment comprises cyclosporin A, Hostapal N-110®, and an oil having a Specific Gravity between 0.90 and 0.95.

Another embodiment comprises cyclosporin A, Igepal CTA-639W®, and an oil having a Specific Gravity between 0.90 and 0.95.

Another embodiment comprises cyclosporin A, Igepal DAP-9®, and an oil having a Specific Gravity between 0.90 and 0.95.

Another embodiment comprises cyclosporin A, Igepal OD-410®, and an oil having a Specific Gravity between 0.90 and 0.95.

Another embodiment comprises cyclosporin A, Igepal SS-837®, and an oil having a Specific Gravity between 0.90 and 0.95.

Another embodiment comprises cyclosporin A, Lipocol NP-9 USP®, and an oil having a Specific Gravity between 0.90 and 0.95.

Another embodiment comprises cyclosporin A, Macol DNP-10®, and an oil having a Specific Gravity between 0.90 and 0.95.

Another embodiment comprises cyclosporin A, Marlophen NP 5®, and an oil having a Specific Gravity between 0.90 and 0.95.

Another embodiment comprises cyclosporin A, Marlophen P 1®, and an oil having a Specific Gravity between 0.90 and 0.95.

Another embodiment comprises cyclosporin A, Surfonic NB®, and an oil having a Specific Gravity between 0.90 and 0.95.

Another embodiment comprises cyclosporin A, Surfonic OPB-307®, and an oil having a Specific Gravity between 0.90 and 0.95.

Another embodiment comprises cyclosporin A, Surfonic OPB-407®, and an oil having a Specific Gravity between 0.90 and 0.95.

Another embodiment comprises cyclosporin A, Syn Fac 334®, and an oil having a Specific Gravity between 0.90 and 0.95.

Another embodiment comprises cyclosporin A, Syn Fac 8216®, and an oil having a Specific Gravity between 0.90 and 0.95.

Another embodiment comprises cyclosporin A, Triton N-57®, and an oil having a Specific Gravity between 0.90 and 0.95.

Another embodiment comprises cyclosporin A, Trycol 6956®, and an oil having a Specific Gravity between 0.90 and 0.95.

Another embodiment comprises cyclosporin A, Trycol 6961®, and an oil having a Specific Gravity between 0.90 and 0.95.

Another embodiment comprises cyclosporin A, Trycol 6964®, and an oil having a Specific Gravity between 0.90 and 0.95.

Another embodiment comprises cyclosporin A, Trycol 6969®, and an oil having a Specific Gravity between 0.90 and 0.95.

Another embodiment comprises cyclosporin A, Trycol 6974®, and an oil having a Specific Gravity between 0.90 and 0.95.

Another embodiment comprises cyclosporin A, Witbreak DRB-127®, and an oil having a Specific Gravity between 0.90 and 0.95.

Another embodiment comprises cyclosporin A, Witbreak DRB-127®, and an oil having a Specific Gravity between 0.90 and 0.95.

Another embodiment comprises cyclosporin A, Witconol NP Series®, and an oil having a Specific Gravity between 0.90 and 0.95.

Another embodiment comprises cyclosporin A, an Ethoxylated Aryl Phenol, and an oil having a Specific Gravity between 0.90 and 0.95.

Another embodiment comprises cyclosporin A, Soprophor BSU®, and an oil having a Specific Gravity between 0.90 and 0.95.

Another embodiment comprises cyclosporin A, Soprophor CY/8®, and an oil having a Specific Gravity between 0.90 and 0.95.

Another embodiment comprises cyclosporin A, Soprophor S/25®, and an oil having a Specific Gravity between 0.90 and 0.95.

Another embodiment comprises cyclosporin A, Witconol NIO®, and an oil having a Specific Gravity between 0.90 and 0.95.

Another embodiment comprises cyclosporin A, Witconol NIW®, and an oil having a Specific Gravity between 0.90 and 0.95.

Another embodiment comprises cyclosporin A, Witconol S-100®, and an oil having a Specific Gravity between 0.90 and 0.95.

Another embodiment comprises cyclosporin A, an Ethoxylated Fatty Acid, and an oil having a Specific Gravity between 0.90 and 0.95.

Another embodiment comprises cyclosporin A, Aldo PGHMS®, and an oil having a Specific Gravity between 0.90 and 0.95.

Another embodiment comprises cyclosporin A, Alkamuls TO-15/HR®, and an oil having a Specific Gravity between 0.90 and 0.95.

Another embodiment comprises cyclosporin A, Armotan AL-69-66®, and an oil having a Specific Gravity between 0.90 and 0.95.

Another embodiment comprises cyclosporin A, Cerasynt 840®, and an oil having a Specific Gravity between 0.90 and 0.95.

Another embodiment comprises cyclosporin A, Cerasynt 945®, and an oil having a Specific Gravity between 0.90 and 0.95.

Another embodiment comprises cyclosporin A, Crystal Inhibitor No. 5®, and an oil having a Specific Gravity between 0.90 and 0.95.

Another embodiment comprises cyclosporin A, DeThox Acid L-9®, and an oil having a Specific Gravity between 0.90 and 0.95.

Another embodiment comprises cyclosporin A, DeThox Acid S-8®, and an oil having a Specific Gravity between 0.90 and 0.95.

Another embodiment comprises cyclosporin A, Ethofat 242/25®, and an oil having a Specific Gravity between 0.90 and 0.95.

Another embodiment comprises cyclosporin A, Hydropalat 65®, and an oil having a Specific Gravity between 0.90 and 0.95.

Another embodiment comprises cyclosporin A, Lipo EGMS®, and an oil having a Specific Gravity between 0.90 and 0.95.

Another embodiment comprises cyclosporin A, Lipopeg 2 DL®, and an oil having a Specific Gravity between 0.90 and 0.95.

Another embodiment comprises cyclosporin A, Lipopeg 4 DL®, and an oil having a Specific Gravity between 0.90 and 0.95.

Another embodiment comprises cyclosporin A, Lipopeg 4-L®, and an oil having a Specific Gravity between 0.90 and 0.95.

Another embodiment comprises cyclosporin A, Lipopeg 39-S®, and an oil having a Specific Gravity between 0.90 and 0.95.

Another embodiment comprises cyclosporin A, Lipopeg 4-S®, and an oil having a Specific Gravity between 0.90 and 0.95.

Another embodiment comprises cyclosporin A, Lipopeg 10-S®, and an oil having a Specific Gravity between 0.90 and 0.95.

Another embodiment comprises cyclosporin A, Lipopeg 100-S®, and an oil having a Specific Gravity between 0.90 and 0.95.

Another embodiment comprises cyclosporin A, Lipopeg 6000 DS®, and an oil having a Specific Gravity between 0.90 and 0.95.

Another embodiment comprises cyclosporin A, Lumulse 40-L®, and an oil having a Specific Gravity between 0.90 and 0.95.

Another embodiment comprises cyclosporin A, Lumulse 40-S®, and an oil having a Specific Gravity between 0.90 and 0.95.

Another embodiment comprises cyclosporin A, Lumulse 42-L®, and an oil having a Specific Gravity between 0.90 and 0.95.

Another embodiment comprises cyclosporin A, Lumulse 42-S®, and an oil having a Specific Gravity between 0.90 and 0.95.

Another embodiment comprises cyclosporin A, Lumulse 100-S®, and an oil having a Specific Gravity between 0.90 and 0.95.

Another embodiment comprises cyclosporin A, Lumulse 602-S®, and an oil having a Specific Gravity between 0.90 and 0.95.

Another embodiment comprises cyclosporin A, Magrabar PGE-20-0®, and an oil having a Specific Gravity between 0.90 and 0.95.

Another embodiment comprises cyclosporin A, Magrabar PGE-20L®, and an oil having a Specific Gravity between 0.90 and 0.95.

Another embodiment comprises cyclosporin A, Magrabar PGE-20T®, and an oil having a Specific Gravity between 0.90 and 0.95.

Another embodiment comprises cyclosporin A, Magrabar PGE-22-0®, and an oil having a Specific Gravity between 0.90 and 0.95.

Another embodiment comprises cyclosporin A, Magrabar PGE-22L®, and an oil having a Specific Gravity between 0.90 and 0.95.

Another embodiment comprises cyclosporin A, Magrabar PGE-22T®, and an oil having a Specific Gravity between 0.90 and 0.95.

Another embodiment comprises cyclosporin A, Magrabar PGE-40-0®, and an oil having a Specific Gravity between 0.90 and 0.95.

Another embodiment comprises cyclosporin A, Magrabar PGE-40L®, and an oil having a Specific Gravity between 0.90 and 0.95.

Another embodiment comprises cyclosporin A, Magrabar PGE-40T®, and an oil having a Specific Gravity between 0.90 and 0.95.

Another embodiment comprises cyclosporin A, Magrabar PGE-42-0®, and an oil having a Specific Gravity between 0.90 and 0.95.

Another embodiment comprises cyclosporin A, Magrabar PGE-42L®, and an oil having a Specific Gravity between 0.90 and 0.95.

Another embodiment comprises cyclosporin A, Magrabar PGE-42T®, and an oil having a Specific Gravity between 0.90 and 0.95.

Another embodiment comprises cyclosporin A, Magrabar PGE-60-0®, and an oil having a Specific Gravity between 0.90 and 0.95.

Another embodiment comprises cyclosporin A, Magrabar PGE-60L®, and an oil having a Specific Gravity between 0.90 and 0.95.

Another embodiment comprises cyclosporin A, Magrabar PGE-60T®, and an oil having a Specific Gravity between 0.90 and 0.95.

Another embodiment comprises cyclosporin A, Magrabar PGE-62-0®, and an oil having a Specific Gravity between 0.90 and 0.95.

Another embodiment comprises cyclosporin A, Magrabar PGE-62L®, and an oil having a Specific Gravity between 0.90 and 0.95.

Another embodiment comprises cyclosporin A, Magrabar PGE-62T®, and an oil having a Specific Gravity between 0.90 and 0.95.

Another embodiment comprises cyclosporin A, Mapeg S-40K®, and an oil having a Specific Gravity between 0.90 and 0.95.

Another embodiment comprises cyclosporin A, Marlowet OTS®, and an oil having a Specific Gravity between 0.90 and 0.95.

Another embodiment comprises cyclosporin A, Naturechem PGR®, and an oil having a Specific Gravity between 0.90 and 0.95.

Another embodiment comprises cyclosporin A, PG No. 4®, and an oil having a Specific Gravity between 0.90 and 0.95.

Another embodiment comprises cyclosporin A, Renex 20®, and an oil having a Specific Gravity between 0.90 and 0.95.

Another embodiment comprises cyclosporin A, Ritox 52®, and an oil having a Specific Gravity between 0.90 and 0.95.

Another embodiment comprises cyclosporin A, Ritox 53®, and an oil having a Specific Gravity between 0.90 and 0.95.

Another embodiment comprises cyclosporin A, Ritox 59®, and an oil having a Specific Gravity between 0.90 and 0.95.

Another embodiment comprises cyclosporin A, Surfax 8916/A®, and an oil having a Specific Gravity between 0.90 and 0.95.

Another embodiment comprises cyclosporin A, Tego Acid S 40 P®, and an oil having a Specific Gravity between 0.90 and 0.95.

Another embodiment comprises cyclosporin A, Tego Acid S 100 P®, and an oil having a Specific Gravity between 0.90 and 0.95.

Another embodiment comprises cyclosporin A, Tween 20®, and an oil having a Specific Gravity between 0.90 and 0.95.

Another embodiment comprises cyclosporin A, Volpo 131®, and an oil having a Specific Gravity between 0.90 and 0.95.

Another embodiment comprises cyclosporin A, an Ethoxylated Fatty Ester or Oil, and an oil having a Specific Gravity between 0.90 and 0.95.

Another embodiment comprises cyclosporin A, Acconon 6-C10®, and an oil having a Specific Gravity between 0.90 and 0.95.

Another embodiment comprises cyclosporin A, Acconon CC-6®, and an oil having a Specific Gravity between 0.90 and 0.95.

Another embodiment comprises cyclosporin A, Acconon CO-7®, and an oil having a Specific Gravity between 0.90 and 0.95.

Another embodiment comprises cyclosporin A, Aldosperse 40/60 FG®, and an oil having a Specific Gravity between 0.90 and 0.95.

Another embodiment comprises cyclosporin A, Aldosperse ML-23®, and an oil having a Specific Gravity between 0.90 and 0.95.

Another embodiment comprises cyclosporin A, Aldosperse MS-20 FG®, and an oil having a Specific Gravity between 0.90 and 0.95.

Another embodiment comprises cyclosporin A, Alkamuls EL-620®, and an oil having a Specific Gravity between 0.90 and 0.95.

Another embodiment comprises cyclosporin A, Alkamuls EL-719®, and an oil having a Specific Gravity between 0.90 and 0.95.

Another embodiment comprises cyclosporin A, Alkamuls EL-985®, and an oil having a Specific Gravity between 0.90 and 0.95.

Another embodiment comprises cyclosporin A, Arlatone G®, and an oil having a Specific Gravity between 0.90 and 0.95.

Another embodiment comprises cyclosporin A, Arlatone T®, and an oil having a Specific Gravity between 0.90 and 0.95.

Another embodiment comprises cyclosporin A, Atlas G-1045A®, and an oil having a Specific Gravity between 0.90 and 0.95.

Another embodiment comprises cyclosporin A, Atlas G-1086®, and an oil having a Specific Gravity between 0.90 and 0.95.

Another embodiment comprises cyclosporin A, Atlas G-1087®, and an oil having a Specific Gravity between 0.90 and 0.95.

Another embodiment comprises cyclosporin A, Atlas G-1089®, and an oil having a Specific Gravity between 0.90 and 0.95.

Another embodiment comprises cyclosporin A, Atlas G-1096®, and an oil having a Specific Gravity between 0.90 and 0.95.

Another embodiment comprises cyclosporin A, Atlas G-1292®, and an oil having a Specific Gravity between 0.90 and 0.95.

Another embodiment comprises cyclosporin A, Atlas G-1293®, and an oil having a Specific Gravity between 0.90 and 0.95.

Another embodiment comprises cyclosporin A, Atlas G-1300®, and an oil having a Specific Gravity between 0.90 and 0.95.

Another embodiment comprises cyclosporin A, Atlas G-7076®, and an oil having a Specific Gravity between 0.90 and 0.95.

Another embodiment comprises cyclosporin A, Capmul EMG®, and an oil having a Specific Gravity between 0.90 and 0.95.

Another embodiment comprises cyclosporin A, Chemonic CO-40®, and an oil having a Specific Gravity between 0.90 and 0.95.

Another embodiment comprises cyclosporin A, Chemonic LI-3®, and an oil having a Specific Gravity between 0.90 and 0.95.

Another embodiment comprises cyclosporin A, Chemonic LI-7®, and an oil having a Specific Gravity between 0.90 and 0.95.

Another embodiment comprises cyclosporin A, Cirrasol GM®, and an oil having a Specific Gravity between 0.90 and 0.95.

Another embodiment comprises cyclosporin A, Cremophor CO 40®, and an oil having a Specific Gravity between 0.90 and 0.95.

Another embodiment comprises cyclosporin A, Cremophor CO 410®, and an oil having a Specific Gravity between 0.90 and 0.95.

Another embodiment comprises cyclosporin A, Cremophor EL®, and an oil having a Specific Gravity between 0.90 and 0.95.

Another embodiment comprises cyclosporin A, Cremophor GC7®, and an oil having a Specific Gravity between 0.90 and 0.95.

Another embodiment comprises cyclosporin A, Cremophor RH-40®, and an oil having a Specific Gravity between 0.90 and 0.95.

Another embodiment comprises cyclosporin A, Crovol A-40®, and an oil having a Specific Gravity between 0.90 and 0.95.

Another embodiment comprises cyclosporin A, Crovol A-70®, and an oil having a Specific Gravity between 0.90 and 0.95.

Another embodiment comprises cyclosporin A, Crovol M-70®, and an oil having a Specific Gravity between 0.90 and 0.95.

Another embodiment comprises cyclosporin A, Crovol PK-70®, and an oil having a Specific Gravity between 0.90 and 0.95.

Another embodiment comprises cyclosporin A, Cutina E-24®, and an oil having a Specific Gravity between 0.90 and 0.95.

Another embodiment comprises cyclosporin A, Dacospin 12-R®, and an oil having a Specific Gravity between 0.90 and 0.95.

Another embodiment comprises cyclosporin A, Dehymuls HRE-7®, and an oil having a Specific Gravity between 0.90 and 0.95.

Another embodiment comprises cyclosporin A, DeSonic 30C®, and an oil having a Specific Gravity between 0.90 and 0.95.

Another embodiment comprises cyclosporin A, DeSonic 36C®, and an oil having a Specific Gravity between 0.90 and 0.95.

Another embodiment comprises cyclosporin A, DeSonic 40C®, and an oil having a Specific Gravity between 0.90 and 0.95.

Another embodiment comprises cyclosporin A, Durfax 60®, and an oil having a Specific Gravity between 0.90 and 0.95.

Another embodiment comprises cyclosporin A, Durfax 65®, and an oil having a Specific Gravity between 0.90 and 0.95.

Another embodiment comprises cyclosporin A, Durfax 80®, and an oil having a Specific Gravity between 0.90 and 0.95.

Another embodiment comprises cyclosporin A, Durfax EOM®, and an oil having a Specific Gravity between 0.90 and 0.95.

Another embodiment comprises cyclosporin A, Eccoterge NF-2®, and an oil having a Specific Gravity between 0.90 and 0.95.

Another embodiment comprises cyclosporin A, Emulpon CO-360®, and an oil having a Specific Gravity between 0.90 and 0.95.

Another embodiment comprises cyclosporin A, Emulpon CO-550®, and an oil having a Specific Gravity between 0.90 and 0.95.

Another embodiment comprises cyclosporin A, Emulsogen EL®, and an oil having a Specific Gravity between 0.90 and 0.95.

Another embodiment comprises cyclosporin A, Emulsogen HCO 040®, and an oil having a Specific Gravity between 0.90 and 0.95.

Another embodiment comprises cyclosporin A, Emulsogen HCO 060®, and an oil having a Specific Gravity between 0.90 and 0.95.

Another embodiment comprises cyclosporin A, Emulsynt 1055l®, and an oil having a Specific Gravity between 0.90 and 0.95.

Another embodiment comprises cyclosporin A, Ethox 3095®, and an oil having a Specific Gravity between 0.90 and 0.95.

Another embodiment comprises cyclosporin A, Eumulgin RO-40®, and an oil having a Specific Gravity between 0.90 and 0.95.

Another embodiment comprises cyclosporin A, Genapol G-260®, and an oil having a Specific Gravity between 0.90 and 0.95.

Another embodiment comprises cyclosporin A, Glycosperse L-20®, and an oil having a Specific Gravity between 0.90 and 0.95.

Another embodiment comprises cyclosporin A, Glycosperse O-5®, and an oil having a Specific Gravity between 0.90 and 0.95.

Another embodiment comprises cyclosporin A, Glycosperse O-20®, and an oil having a Specific Gravity between 0.90 and 0.95.

Another embodiment comprises cyclosporin A, Glycosperse O-20 FG®, and an oil having a Specific Gravity between 0.90 and 0.95.

Another embodiment comprises cyclosporin A, Glycosperse S-20®, and an oil having a Specific Gravity between 0.90 and 0.95.

Another embodiment comprises cyclosporin A, Glycosperse S-20 FG®, and an oil having a Specific Gravity between 0.90 and 0.95.

Another embodiment comprises cyclosporin A, Glycosperse TS-20®, and an oil having a Specific Gravity between 0.90 and 0.95.

Another embodiment comprises cyclosporin A, Glycosperse TS-20 FG®, and an oil having a Specific Gravity between 0.90 and 0.95.

Another embodiment comprises cyclosporin A, Hetan SL®, and an oil having a Specific Gravity between 0.90 and 0.95.

Another embodiment comprises cyclosporin A, Hetan SO®, and an oil having a Specific Gravity between 0.90 and 0.95.

Another embodiment comprises cyclosporin A, Hetan SS®, and an oil having a Specific Gravity between 0.90 and 0.95.

Another embodiment comprises cyclosporin A, Hetoxide C-2®, and an oil having a Specific Gravity between 0.90 and 0.95.

Another embodiment comprises cyclosporin A, Hetoxide C-9®, and an oil having a Specific Gravity between 0.90 and 0.95.

Another embodiment comprises cyclosporin A, Hetoxide C-15®, and an oil having a Specific Gravity between 0.90 and 0.95.

Another embodiment comprises cyclosporin A, Hetoxide C-25®, and an oil having a Specific Gravity between 0.90 and 0.95.

Another embodiment comprises cyclosporin A, Hetoxide C-40®, and an oil having a Specific Gravity between 0.90 and 0.95.

Another embodiment comprises cyclosporin A, Hetoxide C-200®, and an oil having a Specific Gravity between 0.90 and 0.95.

Another embodiment comprises cyclosporin A, Hetoxide C-200-50%®, and an oil having a Specific Gravity between 0.90 and 0.95.

Another embodiment comprises cyclosporin A, Hetoxide GC-30®, and an oil having a Specific Gravity between 0.90 and 0.95.

Another embodiment comprises cyclosporin A, Hetoxide HC-60®, and an oil having a Specific Gravity between 0.90 and 0.95.

Another embodiment comprises cyclosporin A, Ice No. 2®, and an oil having a Specific Gravity between 0.90 and 0.95.

Another embodiment comprises cyclosporin A, Incrocas 30/40®, and an oil having a Specific Gravity between 0.90 and 0.95.

Another embodiment comprises cyclosporin A, Lexol EC®, and an oil having a Specific Gravity between 0.90 and 0.95.

Another embodiment comprises cyclosporin A, Lexol EO®, and an oil having a Specific Gravity between 0.90 and 0.95.

Another embodiment comprises cyclosporin A, Lipocol HCO-40®, and an oil having a Specific Gravity between 0.90 and 0.95.

Another embodiment comprises cyclosporin A, Lipocol HCO-60®, and an oil having a Specific Gravity between 0.90 and 0.95.

Another embodiment comprises cyclosporin A, Lipocol O-3 Special®, and an oil having a Specific Gravity between 0.90 and 0.95.

Another embodiment comprises cyclosporin A, Lipopeg 2-L®, and an oil having a Specific Gravity between 0.90 and 0.95.

Another embodiment comprises cyclosporin A, Lipopeg 4-DO®, and an oil having a Specific Gravity between 0.90 and 0.95.

Another embodiment comprises cyclosporin A, Lipopeg 4-DS®, and an oil having a Specific Gravity between 0.90 and 0.95.

Another embodiment comprises cyclosporin A, Lipovol GTB®, and an oil having a Specific Gravity between 0.90 and 0.95.

Another embodiment comprises cyclosporin A, Lonzest SML-20®, and an oil having a Specific Gravity between 0.90 and 0.95.

Another embodiment comprises cyclosporin A, Lonzest SMO-20®, and an oil having a Specific Gravity between 0.90 and 0.95.

Another embodiment comprises cyclosporin A, Lonzest SMS-20®, and an oil having a Specific Gravity between 0.90 and 0.95.

Another embodiment comprises cyclosporin A, Lonzest STO-20®, and an oil having a Specific Gravity between 0.90 and 0.95.

Another embodiment comprises cyclosporin A, Lonzest STS-20®, and an oil having a Specific Gravity between 0.90 and 0.95.

Another embodiment comprises cyclosporin A, Lumulse GR-40®, and an oil having a Specific Gravity between 0.90 and 0.95.

Another embodiment comprises cyclosporin A, Lumulse GRH-40®, and an oil having a Specific Gravity between 0.90 and 0.95.

Another embodiment comprises cyclosporin A, Lumulse POE (7) GML®, and an oil having a Specific Gravity between 0.90 and 0.95.

Another embodiment comprises cyclosporin A, Lumulse POE (12) Glyc®, and an oil having a Specific Gravity between 0.90 and 0.95.

Another embodiment comprises cyclosporin A, Lumulse POE (40) MS KP®, and an oil having a Specific Gravity between 0.90 and 0.95.

Another embodiment comprises cyclosporin A, Marlowet 4750®, and an oil having a Specific Gravity between 0.90 and 0.95.

Another embodiment comprises cyclosporin A, Marlowet LVS®, and an oil having a Specific Gravity between 0.90 and 0.95.

Another embodiment comprises cyclosporin A, Marlowet R 11®, and an oil having a Specific Gravity between 0.90 and 0.95.

Another embodiment comprises cyclosporin A, Marlowet R 40®, and an oil having a Specific Gravity between 0.90 and 0.95.

Another embodiment comprises cyclosporin A, Mazol 80 MGK®, and an oil having a Specific Gravity between 0.90 and 0.95.

Another embodiment comprises cyclosporin A, Nonionic Emulsifier T-9®, and an oil having a Specific Gravity between 0.90 and 0.95.

Another embodiment comprises cyclosporin A, Oronal LCG®, and an oil having a Specific Gravity between 0.90 and 0.95.

Another embodiment comprises cyclosporin A, Polyderm PPI-CO-200®, and an oil having a Specific Gravity between 0.90 and 0.95.

Another embodiment comprises cyclosporin A, Polyderm PPI-CO-40®, and an oil having a Specific Gravity between 0.90 and 0.95.

Another embodiment comprises cyclosporin A, Rewoderm LI 520-70®, and an oil having a Specific Gravity between 0.90 and 0.95.

Another embodiment comprises cyclosporin A, Ritapeg 150 DS®, and an oil having a Specific Gravity between 0.90 and 0.95.

Another embodiment comprises cyclosporin A, Softigen 767®, and an oil having a Specific Gravity between 0.90 and 0.95.

Another embodiment comprises cyclosporin A, Surfactol 318®, and an oil having a Specific Gravity between 0.90 and 0.95.

Another embodiment comprises cyclosporin A, Surfactol 365®, and an oil having a Specific Gravity between 0.90 and 0.95.

Another embodiment comprises cyclosporin A, Syn Lube 107®, and an oil having a Specific Gravity between 0.90 and 0.95.

Another embodiment comprises cyclosporin A, Syn Lube 728®, and an oil having a Specific Gravity between 0.90 and 0.95.

Another embodiment comprises cyclosporin A, Syn Lube 1632H®, and an oil having a Specific Gravity between 0.90 and 0.95.

Another embodiment comprises cyclosporin A, Syn Lube 6277-A®, and an oil having a Specific Gravity between 0.90 and 0.95.

Another embodiment comprises cyclosporin A, T-Det C-20®, and an oil having a Specific Gravity between 0.90 and 0.95.

Another embodiment comprises cyclosporin A, T-Det C-40®, and an oil having a Specific Gravity between 0.90 and 0.95.

Another embodiment comprises cyclosporin A, Tally 100 Plus®, and an oil having a Specific Gravity between 0.90 and 0.95.

Another embodiment comprises cyclosporin A, Uniperol EL®, and an oil having a Specific Gravity between 0.90 and 0.95.

Another embodiment comprises cyclosporin A, a Fatty Ester, and an oil having a Specific Gravity between 0.90 and 0.95.

Another embodiment comprises cyclosporin A, Actralube-Syn 147®, and an oil having a Specific Gravity between 0.90 and 0.95.

Another embodiment comprises cyclosporin A, Atlas G-1556®, and an oil having a Specific Gravity between 0.90 and 0.95.

Another embodiment comprises cyclosporin A, Atlas G-1564®, and an oil having a Specific Gravity between 0.90 and 0.95.

Another embodiment comprises cyclosporin A, Atlasol Base Oil S®, and an oil having a Specific Gravity between 0.90 and 0.95.

Another embodiment comprises cyclosporin A, Base ML®, and an oil having a Specific Gravity between 0.90 and 0.95.

Another embodiment comprises cyclosporin A, Base MT®, and an oil having a Specific Gravity between 0.90 and 0.95.

Another embodiment comprises cyclosporin A, Cerasynt 303®, and an oil having a Specific Gravity between 0.90 and 0.95.

Another embodiment comprises cyclosporin A, Dermol 1012®, and an oil having a Specific Gravity between 0.90 and 0.95.

Another embodiment comprises cyclosporin A, Kemester 4000®, and an oil having a Specific Gravity between 0.90 and 0.95.

Another embodiment comprises cyclosporin A, Lactipol S®, and an oil having a Specific Gravity between 0.90 and 0.95.

Another embodiment comprises cyclosporin A, Magrabar PGO®, and an oil having a Specific Gravity between 0.90 and 0.95.

Another embodiment comprises cyclosporin A, Mayco Base BFO®, and an oil having a Specific Gravity between 0.90 and 0.95.

Another embodiment comprises cyclosporin A, Methyl Linoleate®, and an oil having a Specific Gravity between 0.90 and 0.95.

Another embodiment comprises cyclosporin A, Pationic 122A®, and an oil having a Specific Gravity between 0.90 and 0.95.

Another embodiment comprises cyclosporin A, Pationic 138C®, and an oil having a Specific Gravity between 0.90 and 0.95.

Another embodiment comprises cyclosporin A, Pationic CSL®, and an oil having a Specific Gravity between 0.90 and 0.95.

Another embodiment comprises cyclosporin A, Pationic ISL®, and an oil having a Specific Gravity between 0.90 and 0.95.

Another embodiment comprises cyclosporin A, Pationic SBL®, and an oil having a Specific Gravity between 0.90 and 0.95.

Another embodiment comprises cyclosporin A, Pationic SSL®, and an oil having a Specific Gravity between 0.90 and 0.95.

Another embodiment comprises cyclosporin A, Ritasol®, and an oil having a Specific Gravity between 0.90 and 0.95.

Another embodiment comprises cyclosporin A, Tego Alkanol CS 20®, and an oil having a Specific Gravity between 0.90 and 0.95.

Another embodiment comprises cyclosporin A, Tego Alkanol L23 P®, and an oil having a Specific Gravity between 0.90 and 0.95.

Another embodiment comprises cyclosporin A, Tego Alkanol S2®, and an oil having a Specific Gravity between 0.90 and 0.95.

Another embodiment comprises cyclosporin A, Tego Alkanol S20 P®, and an oil having a Specific Gravity between 0.90 and 0.95.

Another embodiment comprises cyclosporin A, Triemulsifier 600 MS®, and an oil having a Specific Gravity between 0.90 and 0.95.

Another embodiment comprises cyclosporin A, a Glycerol Ester, and an oil having a Specific Gravity between 0.90 and 0.95.

Another embodiment comprises cyclosporin A, Agro #9 Wint SBO®, and an oil having a Specific Gravity between 0.90 and 0.95.

Another embodiment comprises cyclosporin A, Ahcovel Base 700®, and an oil having a Specific Gravity between 0.90 and 0.95.

Another embodiment comprises cyclosporin A, Aldo HMS FG®, and an oil having a Specific Gravity between 0.90 and 0.95.

Another embodiment comprises cyclosporin A, Aldo MLD®, and an oil having a Specific Gravity between 0.90 and 0.95.

Another embodiment comprises cyclosporin A, Aldo MLD FG®, and an oil having a Specific Gravity between 0.90 and 0.95.

Another embodiment comprises cyclosporin A, Aldo MO FG®, and an oil having a Specific Gravity between 0.90 and 0.95.

Another embodiment comprises cyclosporin A, Aldo MS®, and an oil having a Specific Gravity between 0.90 and 0.95.

Another embodiment comprises cyclosporin A, Aldo MS FG®, and an oil having a Specific Gravity between 0.90 and 0.95.

Another embodiment comprises cyclosporin A, Aldo MS LG FG®, and an oil having a Specific Gravity between 0.90 and 0.95.

Another embodiment comprises cyclosporin A, Aldo MSD®, and an oil having a Specific Gravity between 0.90 and 0.95.

Another embodiment comprises cyclosporin A, Aldo MSD FG®, and an oil having a Specific Gravity between 0.90 and 0.95.

Another embodiment comprises cyclosporin A, Aldosperse O-20 FG®, and an oil having a Specific Gravity between 0.90 and 0.95.

Another embodiment comprises cyclosporin A, Aldosperse TS-20 FG®, and an oil having a Specific Gravity between 0.90 and 0.95.

Another embodiment comprises cyclosporin A, Aldosperse TS-40 FG®, and an oil having a Specific Gravity between 0.90 and 0.95.

Another embodiment comprises cyclosporin A, Arlacel 165®, and an oil having a Specific Gravity between 0.90 and 0.95.

Another embodiment comprises cyclosporin A, Arlacel 186®, and an oil having a Specific Gravity between 0.90 and 0.95.

Another embodiment comprises cyclosporin A, Capmul GMO®, and an oil having a Specific Gravity between 0.90 and 0.95.

Another embodiment comprises cyclosporin A, Capmul GMS®, and an oil having a Specific Gravity between 0.90 and 0.95.

Another embodiment comprises cyclosporin A, Caprol 3GO®, and an oil having a Specific Gravity between 0.90 and 0.95.

Another embodiment comprises cyclosporin A, Caprol 3GVS®, and an oil having a Specific Gravity between 0.90 and 0.95.

Another embodiment comprises cyclosporin A, Caprol 6G2S®, and an oil having a Specific Gravity between 0.90 and 0.95.

Another embodiment comprises cyclosporin A, Caprol 10G40®, and an oil having a Specific Gravity between 0.90 and 0.95.

Another embodiment comprises cyclosporin A, Caprol 10G100®, and an oil having a Specific Gravity between 0.90 and 0.95.

Another embodiment comprises cyclosporin A, Caprol ET®, and an oil having a Specific Gravity between 0.90 and 0.95.

Another embodiment comprises cyclosporin A, Caprol PGE860®, and an oil having a Specific Gravity between 0.90 and 0.95.

Another embodiment comprises cyclosporin A, Cerasynt 945®, and an oil having a Specific Gravity between 0.90 and 0.95.

Another embodiment comprises cyclosporin A, Cerasynt GMS®, and an oil having a Specific Gravity between 0.90 and 0.95.

Another embodiment comprises cyclosporin A, Cerasynt Q®, and an oil having a Specific Gravity between 0.90 and 0.95.

Another embodiment comprises cyclosporin A, Cerasynt SD®, and an oil having a Specific Gravity between 0.90 and 0.95.

Another embodiment comprises cyclosporin A, Cerasynt WM®, and an oil having a Specific Gravity between 0.90 and 0.95.

Another embodiment comprises cyclosporin A, Chemsperse 14®, and an oil having a Specific Gravity between 0.90 and 0.95.

Another embodiment comprises cyclosporin A, Cremophor GO-32®, and an oil having a Specific Gravity between 0.90 and 0.95.

Another embodiment comprises cyclosporin A, Cremophor GS11®, and an oil having a Specific Gravity between 0.90 and 0.95.

Another embodiment comprises cyclosporin A, Cremophor GS-32®, and an oil having a Specific Gravity between 0.90 and 0.95.

Another embodiment comprises cyclosporin A, Cutina KD-16®, and an oil having a Specific Gravity between 0.90 and 0.95.

Another embodiment comprises cyclosporin A, Dehymuls PGPH®, and an oil having a Specific Gravity between 0.90 and 0.95.

Another embodiment comprises cyclosporin A, Dermol DGDIS®, and an oil having a Specific Gravity between 0.90 and 0.95.

Another embodiment comprises cyclosporin A, Dermol DGMIS®, and an oil having a Specific Gravity between 0.90 and 0.95.

Another embodiment comprises cyclosporin A, Dermol G-76®, and an oil having a Specific Gravity between 0.90 and 0.95.

Another embodiment comprises cyclosporin A, Dermol G-7DI®, and an oil having a Specific Gravity between 0.90 and 0.95.

Another embodiment comprises cyclosporin A, Dermol NGDI®, and an oil having a Specific Gravity between 0.90 and 0.95.

Another embodiment comprises cyclosporin A, Dermolan GLH®, and an oil having a Specific Gravity between 0.90 and 0.95.

Another embodiment comprises cyclosporin A, Drewmulse GMO®, and an oil having a Specific Gravity between 0.90 and 0.95.

Another embodiment comprises cyclosporin A, Drewpol 3-5-M®, and an oil having a Specific Gravity between 0.90 and 0.95.

Another embodiment comprises cyclosporin A, Durlac 100 W®, and an oil having a Specific Gravity between 0.90 and 0.95.

Another embodiment comprises cyclosporin A, Dur-Lo®, and an oil having a Specific Gravity between 0.90 and 0.95.

Another embodiment comprises cyclosporin A, Dynasan 118®, and an oil having a Specific Gravity between 0.90 and 0.95.

Another embodiment comprises cyclosporin A, EC-25®, and an oil having a Specific Gravity between 0.90 and 0.95.

Another embodiment comprises cyclosporin A, EM 40®, and an oil having a Specific Gravity between 0.90 and 0.95.

Another embodiment comprises cyclosporin A, Emerest 2400®, and an oil having a Specific Gravity between 0.90 and 0.95.

Another embodiment comprises cyclosporin A, Emerest 2452®, and an oil having a Specific Gravity between 0.90 and 0.95.

Another embodiment comprises cyclosporin A, Empilan G-26®, and an oil having a Specific Gravity between 0.90 and 0.95.

Another embodiment comprises cyclosporin A, Genapol TSM®, and an oil having a Specific Gravity between 0.90 and 0.95.

Another embodiment comprises cyclosporin A, Hostacerin DGI®, and an oil having a Specific Gravity between 0.90 and 0.95.

Another embodiment comprises cyclosporin A, Hostacerin DGL®, and an oil having a Specific Gravity between 0.90 and 0.95.

Another embodiment comprises cyclosporin A, Hostacerin DGMS®, and an oil having a Specific Gravity between 0.90 and 0.95.

Another embodiment comprises cyclosporin A, Hostacerin DGSB®, and an oil having a Specific Gravity between 0.90 and 0.95.

Another embodiment comprises cyclosporin A, Ice No. 2®, and an oil having a Specific Gravity between 0.90 and 0.95.

Another embodiment comprises cyclosporin A, Imwitor 742®, and an oil having a Specific Gravity between 0.90 and 0.95.

Another embodiment comprises cyclosporin A, Imwitor 780 K®, and an oil having a Specific Gravity between 0.90 and 0.95.

Another embodiment comprises cyclosporin A, Imwitor 960 Flakes®, and an oil having a Specific Gravity between 0.90 and 0.95.

Another embodiment comprises cyclosporin A, Isolan GI 34®, and an oil having a Specific Gravity between 0.90 and 0.95.

Another embodiment comprises cyclosporin A, isolan GO 33®, and an oil having a Specific Gravity between 0.90 and 0.95.

Another embodiment comprises cyclosporin A, Kemester 1000®, and an oil having a Specific Gravity between 0.90 and 0.95.

Another embodiment comprises cyclosporin A, Kemester 2000®, and an oil having a Specific Gravity between 0.90 and 0.95.

Another embodiment comprises cyclosporin A, Kemester 2000®, and an oil having a Specific Gravity between 0.90 and 0.95.

Another embodiment comprises cyclosporin A, Kemester 6000SE®, and an oil having a Specific Gravity between 0.90 and 0.95.

Another embodiment comprises cyclosporin A, Lamecreme DGE 18®, and an.oil having a Specific Gravity between 0.90 and 0.95.

Another embodiment comprises cyclosporin A, Lexemul 515®, and an oil having a Specific Gravity between 0.90 and 0.95.

Another embodiment comprises cyclosporin A, Lexemul 561®, and an oil having a Specific Gravity between 0.90 and 0.95.

Another embodiment comprises cyclosporin A, Lexemul AR®, and an oil having a Specific Gravity between 0.90 and 0.95.

Another embodiment comprises cyclosporin A, Lexemul AS®, and an oil having a Specific Gravity between 0.90 and 0.95.

Another embodiment comprises cyclosporin A, Lexemul GDL®, and an oil having a Specific Gravity between 0.90 and 0.95.

Another embodiment comprises cyclosporin A, Lexemul T®, and an oil having a Specific Gravity between 0.90 and 0.95.

Another embodiment comprises cyclosporin A, Lipomulse 165®, and an oil having a Specific Gravity between 0.90 and 0.95.

Another embodiment comprises cyclosporin A, Lumulse GML K®, and an oil having a Specific Gravity between 0.90 and 0.95.

Another embodiment comprises cyclosporin A, Lumulse GMO K®, and an oil having a Specific Gravity between 0.90 and 0.95.

Another embodiment comprises cyclosporin A, Lumulse GMR K®, and an oil having a Specific Gravity between 0.90 and 0.95.

Another embodiment comprises cyclosporin A, Lumulse GMT K®, and an oil having a Specific Gravity between 0.90 and 0.95.

Another embodiment comprises cyclosporin A, Magrabar GMC®, and an oil having a Specific Gravity between 0.90 and 0.95.

Another embodiment comprises cyclosporin A, Magrabar GMO-CK®, and an oil having a Specific Gravity between 0.90 and 0.95.

Another embodiment comprises cyclosporin A, Magrabar MDG-5050®, and an oil having a Specific Gravity between 0.90 and 0.95.

Another embodiment comprises cyclosporin A, Magrabar PGO-315®, and an oil having a Specific Gravity between 0.90 and 0.95.

Another embodiment comprises cyclosporin A, Magrabar PGO-1010®, and an oil having a Specific Gravity between 0.90 and 0.95.

Another embodiment comprises cyclosporin A, Mazol 300K®, and an oil having a Specific Gravity between 0.90 and 0.95.

Another embodiment comprises cyclosporin A, Mazol GMO-K®, and an oil having a Specific Gravity between 0.90 and 0.95.

Another embodiment comprises cyclosporin A, Mazol GMS-K®, and an oil having a Specific Gravity between 0.90 and 0.95.

Another embodiment comprises cyclosporin A, Mazol PG031-K®, and an oil having a Specific Gravity between 0.90 and 0.95.

Another embodiment comprises cyclosporin A, Miglyol 812®, and an oil having a Specific Gravity between 0.90 and 0.95.

Another embodiment comprises cyclosporin A, Norfox 165C®, and an oil having a Specific Gravity between 0.90 and 0.95.

Another embodiment comprises cyclosporin A, Schercemol GMIS®, and an oil having a Specific Gravity between 0.90 and 0.95.

Another embodiment comprises cyclosporin A, Tegin®, and an oil having a Specific Gravity between 0.90 and 0.95.

Another embodiment comprises cyclosporin A, Tegin 4100 Pellets®, and an oil having a Specific Gravity between 0.90 and 0.95.

Another embodiment comprises cyclosporin A, Tegin M Pellets®, and an oil having a Specific Gravity between 0.90 and 0.95.

Another embodiment comprises cyclosporin A, Tegin OV®, and an oil having a Specific Gravity between 0.90 and 0.95.

Another embodiment comprises cyclosporin A, Teginacid H®, and an oil having a Specific Gravity between 0.90 and 0.95.

Another embodiment comprises cyclosporin A, Tego Cosmo P813®, and an oil having a Specific Gravity between 0.90 and 0.95.

Another embodiment comprises cyclosporin A, Wickenol 535®, and an oil having a Specific Gravity between 0.90 and 0.95.

Another embodiment comprises cyclosporin A, Witconol 14®, and an oil having a Specific Gravity between 0.90 and 0.95.

Another embodiment comprises cyclosporin A, Witconol 14®, and an oil having a Specific Gravity between 0.90 and 0.95.

Another embodiment comprises cyclosporin A, Witconol 14F®, and an oil having a Specific Gravity between 0.90 and 0.95.

Another embodiment comprises cyclosporin A, Witconol 18L®, and an oil having a Specific Gravity between 0.90 and 0.95.

Another embodiment comprises cyclosporin A, Witconol GOT®, and an oil having a Specific Gravity between 0.90 and 0.95.

Another embodiment comprises cyclosporin A, Witconol MST®, and an oil having a Specific Gravity between 0.90 and 0.95.

Another embodiment comprises cyclosporin A, Witconol RHT®, and an oil having a Specific Gravity between 0.90 and 0.95.

Another embodiment comprises cyclosporin A, a Glycol Ester, and an oil having a Specific Gravity between 0.90 and 0.95.

Another embodiment comprises cyclosporin A, Alkamuls 600 DO®, and an oil having a Specific Gravity between 0.90 and 0.95.

Another embodiment comprises cyclosporin A, Alkamuls SEG®, and an oil having a Specific Gravity between 0.90 and 0.95.

Another embodiment comprises cyclosporin A, Atlas EM-2®, and an oil having a Specific Gravity between 0.90 and 0.95.

Another embodiment comprises cyclosporin A, Cerasynt IP®, and an oil having a Specific Gravity between 0.90 and 0.95.

Another embodiment comprises cyclosporin A, Cerasynt M®, and an oil having a Specific Gravity between 0.90 and 0.95.

Another embodiment comprises cyclosporin A, Cerasynt MN®, and an oil having a Specific Gravity between 0.90 and 0.95.

Another embodiment comprises cyclosporin A, Cerasynt PA®, and an oil having a Specific Gravity between 0.90 and 0.95.

Another embodiment comprises cyclosporin A, Chemsperse EGDS®, and an oil having a Specific Gravity between 0.90 and 0.95.

Another embodiment comprises cyclosporin A, Chemsperse EGMS®, and an oil having a Specific Gravity between 0.90 and 0.95.

Another embodiment comprises cyclosporin A, Colonial Monolaurin®, and an oil having a Specific Gravity between 0.90 and 0.95.

Another embodiment comprises cyclosporin A, DeMuls SGE-95®, and an oil having a Specific Gravity between 0.90 and 0.95.

Another embodiment comprises cyclosporin A, Eccoterge 200®, and an oil having a Specific Gravity between 0.90 and 0.95.

Another embodiment comprises cyclosporin A, Emerest 2380®, and an oil having a Specific Gravity between 0.90 and 0.95.

Another embodiment comprises cyclosporin A, Ethox 2610®, and an oil having a Specific Gravity between 0.90 and 0.95.

Another embodiment comprises cyclosporin A, Ethox DO-9®, and an oil having a Specific Gravity between 0.90 and 0.95.

Another embodiment comprises cyclosporin A, Ethox DO-14®, and an oil having a Specific Gravity between 0.90 and 0.95.

Another embodiment comprises cyclosporin A, Ethox SO-9®, and an oil having a Specific Gravity between 0.90 and 0.95.

Another embodiment comprises cyclosporin A, Fizul MD-318®, and an oil having a Specific Gravity between 0.90 and 0.95.

Another embodiment comprises cyclosporin A, Genapol EGDS-VHP®, and an oil having a Specific Gravity between 0.90 and 0.95.

Another embodiment comprises cyclosporin A, Genapol TS Powder®, and an oil having a Specific Gravity between 0.90 and 0.95.

Another embodiment comprises cyclosporin A, Hostacerin WO®, and an oil having a Specific Gravity between 0.90 and 0.95.

Another embodiment comprises cyclosporin A, Inversol 140®, and an oil having a Specific Gravity between 0.90 and 0.95.

Another embodiment comprises cyclosporin A, Kemester 104®, and an oil having a Specific Gravity between 0.90 and 0.95.

Another embodiment comprises cyclosporin A, Kemester 205®, and an oil having a Specific Gravity between 0.90 and 0.95.

Another embodiment comprises cyclosporin A, Kemester 226®, and an oil having a Specific Gravity between 0.90 and 0.95.

Another embodiment comprises cyclosporin A, Kemester 5221SE®, and an oil having a Specific Gravity between 0.90 and 0.95.

Another embodiment comprises cyclosporin A, Kemester EGDS®, and an oil having a Specific Gravity between 0.90 and 0.95.

Another embodiment comprises cyclosporin A, Lexemul EGDS®, and an oil having a Specific Gravity between 0.90 and 0.95.

Another embodiment comprises cyclosporin A, Lexemul EGMS®, and an oil having a Specific Gravity between 0.90 and 0.95.

Another embodiment comprises cyclosporin A, Lexemul P®, and an oil having a Specific Gravity between 0.90 and 0.95.

Another embodiment comprises cyclosporin A, Lipo DGLS®, and an oil having a Specific Gravity between 0.90 and 0.95.

Another embodiment comprises cyclosporin A, Lipo EGDS®, and an oil having a Specific Gravity between 0.90 and 0.95.

Another embodiment comprises cyclosporin A, Lipo PGMS®, and an oil having a Specific Gravity between 0.90 and 0.95.

Another embodiment comprises cyclosporin A, Liposorb S-4®, and an oil having a Specific Gravity between 0.90 and 0.95.

Another embodiment comprises cyclosporin A, Liposorb TO-20®, and an oil having a Specific Gravity between 0.90 and 0.95.

Another embodiment comprises cyclosporin A, Lumulse PGO®, and an oil having a Specific Gravity between 0.90 and 0.95.

Another embodiment comprises cyclosporin A, Mackester EGDS®, and an oil having a Specific Gravity between 0.90 and 0.95.

Another embodiment comprises cyclosporin A, Mackester EGMS®, and an oil having a Specific Gravity between 0.90 and 0.95.

Another embodiment comprises cyclosporin A, Mackester GSTP®, and an oil having a Specific Gravity between 0.90 and 0.95.

Another embodiment comprises cyclosporin A, Mackester Series®, and an oil having a Specific Gravity between 0.90 and 0.95.

Another embodiment comprises cyclosporin A, Magrabar PDG-50®, and an oil having a Specific Gravity between 0.90 and 0.95.

Another embodiment comprises cyclosporin A, Mapeg 6000 DS®, and an oil having a Specific Gravity between 0.90 and 0.95.

Another embodiment comprises cyclosporin A, Marlowet 4702®, and an oil having a Specific Gravity between 0.90 and 0.95.

Another embodiment comprises cyclosporin A, Monalube 305®, and an oil having a Specific Gravity between 0.90 and 0.95.

Another embodiment comprises cyclosporin A, Monalube 310®, and an oil having a Specific Gravity between 0.90 and 0.95.

Another embodiment comprises cyclosporin A, Monalube 315®, and an oil having a Specific Gravity between 0.90 and 0.95.

Another embodiment comprises cyclosporin A, Monalube 320®, and an oil having a Specific Gravity between 0.90 and 0.95.

Another embodiment comprises cyclosporin A, Monalube 325®, and an oil having a Specific Gravity between 0.90 and 0.95.

Another embodiment comprises cyclosporin A, Monalube 330®, and an oil having a Specific Gravity between 0.90 and 0.95.

Another embodiment comprises cyclosporin A, Naturechem PGHS®, and an oil having a Specific Gravity between 0.90 and 0.95.

Another embodiment comprises cyclosporin A, Polycastorol PLO-840®, and an oil having a Specific Gravity between 0.90 and 0.95.

Another embodiment comprises cyclosporin A, Polytex 10M®, and an oil having a Specific Gravity between 0.90 and 0.95.

Another embodiment comprises cyclosporin A, Ritasynt IP®, and an oil having a Specific Gravity between 0.90 and 0.95.

Another embodiment comprises cyclosporin A, Ross Chem PEG 600 DT®, and an oil having a Specific Gravity between 0.90 and 0.95.

Another embodiment comprises cyclosporin A, Schercemol PGMS®, and an oil having a Specific Gravity between 0.90 and 0.95.

Another embodiment comprises cyclosporin A, Sponto H-44C®, and an oil having a Specific Gravity between 0.90 and 0.95.

Another embodiment comprises cyclosporin A, Tegin G®, and an oil having a Specific Gravity between 0.90 and 0.95.

Another embodiment comprises cyclosporin A, Witbreak DGE-182®, and an oil having a Specific Gravity between 0.90 and 0.95.

Another embodiment comprises cyclosporin A, Witbreak DGE-182®, and an oil having a Specific Gravity between 0.90 and 0.95.

Another embodiment comprises cyclosporin A, Witbreak DRA-21®, and an oil having a Specific Gravity between 0.90 and 0.95.

Another embodiment comprises cyclosporin A, Witbreak DRA-21®, and an oil having a Specific Gravity between 0.90 and 0.95.

Another embodiment comprises cyclosporin A, Witbreak DRA-50®, and an oil having a Specific Gravity between 0.90 and 0.95.

Another embodiment comprises cyclosporin A, Witbreak DRA-50®, and an oil having a Specific Gravity between 0.90 and 0.95.

Another embodiment comprises cyclosporin A, Witconol F26-46®, and an oil having a Specific Gravity between 0.90 and 0.95.

Another embodiment comprises cyclosporin A, Witconol H-32®, and an oil having a Specific Gravity between 0.90 and 0.95.

Another embodiment comprises cyclosporin A, Witconol H-33®, and an oil having a Specific Gravity between 0.90 and 0.95.

Another embodiment comprises cyclosporin A, Witconol H-35A®, and an oil having a Specific Gravity between 0.90 and 0.95.

Another embodiment comprises cyclosporin A, Witconol RHP®, and an oil having a Specific Gravity between 0.90 and 0.95.

Another embodiment comprises cyclosporin A, a Lanolin-based Derivative, and an oil having a Specific Gravity between 0.90 and 0.95.

Another embodiment comprises cyclosporin A, Amerchol CAB®, and an oil having a Specific Gravity between 0.90 and 0.95.

Another embodiment comprises cyclosporin A, Amerchol L-101®, and an oil having a Specific Gravity between 0.90 and 0.95.

Another embodiment comprises cyclosporin A, Amerlate LFA-LO®, and an oil having a Specific Gravity between 0.90 and 0.95.

Another embodiment comprises cyclosporin A, Amerlate P®, and an oil having a Specific Gravity between 0.90 and 0.95.

Another embodiment comprises cyclosporin A, Barre Common Degras®, and an oil having a Specific Gravity between 0.90 and 0.95.

Another embodiment comprises cyclosporin A, Cholesterol NF®, and an oil having a Specific Gravity between 0.90 and 0.95.

Another embodiment comprises cyclosporin A, Crodalan AWS®, and an oil having a Specific Gravity between 0.90 and 0.95.

Another embodiment comprises cyclosporin A, Crodalan LA®, and an oil having a Specific Gravity between 0.90 and 0.95.

Another embodiment comprises cyclosporin A, Emery 1650®, and an oil having a Specific Gravity between 0.90 and 0.95.

Another embodiment comprises cyclosporin A, Emery 1650®, and an oil having a Specific Gravity between 0.90 and 0.95.

Another embodiment comprises cyclosporin A, Emery 1740®, and an oil having a Specific Gravity between 0.90 and 0.95.

Another embodiment comprises cyclosporin A, Emery 1740®, and an oil having a Specific Gravity between 0.90 and 0.95.

Another embodiment comprises cyclosporin A, Forlan 500®, and an oil having a Specific Gravity between 0.90 and 0.95.

Another embodiment comprises cyclosporin A, Forlan L®, and an oil having a Specific Gravity between 0.90 and 0.95.

Another embodiment comprises cyclosporin A, Laneto 50®, and an oil having a Specific Gravity between 0.90 and 0.95.

Another embodiment comprises cyclosporin A, Laneto 100®, and an oil having a Specific Gravity between 0.90 and 0.95.

Another embodiment comprises cyclosporin A, Laneto AWS®, and an oil having a Specific Gravity between 0.90 and 0.95.

Another embodiment comprises cyclgsporin A, Lanfrax 1776®, and an oil having a Specific Gravity between 0.90 and 0.95.

Another embodiment comprises cyclosporin A, Lanfrax 1776®, and an oil having a Specific Gravity between 0.90 and 0.95.

Another embodiment comprises cyclosporin A, Lanogel 21®, and an oil having a Specific Gravity between 0.90 and 0.95.

Another embodiment comprises cyclosporin A, Lipolan®, and an oil having a Specific Gravity between 0.90 and 0.95.

Another embodiment comprises cyclosporin A, Lipolan 31®, and an oil having a Specific Gravity between 0.90 and 0.95.

Another embodiment comprises cyclosporin A, OHlan®, and an oil having a Specific Gravity between 0.90 and 0.95.

Another embodiment comprises cyclosporin A, Polychol 5®, and an oil having a Specific Gravity between 0.90 and 0.95.

Another embodiment comprises cyclosporin A, Polychol 15®, and an oil having a Specific Gravity between 0.90 and 0.95.

Another embodiment comprises cyclosporin A, Ritacetyl®, and an oil having a Specific Gravity between 0.90 and 0.95.

Another embodiment comprises cyclosporin A, Ritachol®, and an oil having a Specific Gravity between 0.90 and 0.95.

Another embodiment comprises cyclosporin A, Ritahydrox®, and an oil having a Specific Gravity between 0.90 and 0.95.

Another embodiment comprises cyclosporin A, Ritalafa®, and an oil having a Specific Gravity between 0.90 and 0.95.

Another embodiment comprises cyclosporin A, Ritalan®, and an oil having a Specific Gravity between 0.90 and 0.95.

Another embodiment comprises cyclosporin A, Ritalan AWS®, and an oil having a Specific Gravity between 0.90 and 0.95.

Another embodiment comprises cyclosporin A, Ritalan C®, and an oil having a Specific Gravity between 0.90 and 0.95.

Another embodiment comprises cyclosporin A, Ritawax®, and an oil having a Specific Gravity between 0.90 and 0.95.

Another embodiment comprises cyclosporin A, Ritawax AEO®, and an oil having a Specific Gravity between 0.90 and 0.95.

Another embodiment comprises cyclosporin A, Ritawax ALA®, and an oil having a Specific Gravity between 0.90 and 0.95.

Another embodiment comprises cyclosporin A, Solan/Solan 50/Super Solan®, and an oil having a Specific Gravity between 0.90 and 0.95.

Another embodiment comprises cyclosporin A, Super Hartolan/Hartolan®, and an oil having a Specific Gravity between 0.90 and 0.95.

Another embodiment comprises cyclosporin A, Supersat AWS-4®, and an oil having a Specific Gravity between 0.90 and 0.95.

Another embodiment comprises cyclosporin A, Supersat AWS-24®, and an oil having a Specific Gravity between 0.90 and 0.95.

Another embodiment comprises cyclosporin A, a Lecithin or Lecithin Derivative, and an oil having a Specific Gravity between 0.90 and 0.95.

Another embodiment comprises cyclosporin A, Alcolec®, and an oil having a Specific Gravity between 0.90 and 0.95.

Another embodiment comprises cyclosporin A, Lecithin®, and an oil having a Specific Gravity between 0.90 and 0.95.

Another embodiment comprises cyclosporin A, Lexin K®, and an oil having a Specific Gravity between 0.90 and 0.95.

Another embodiment comprises cyclosporin A, Natipide®, and an oil having a Specific Gravity between 0.90 and 0.95.

Another embodiment comprises cyclosporin A, a Lignin or Lignin Derivative, and an oil having a Specific Gravity between 0.90 and 0.95.

Another embodiment comprises cyclosporin A, Diwatex XP 9®, and an oil having a Specific Gravity between 0.90 and 0.95.

Another embodiment comprises cyclosporin A, Dynasperse LCD®, and an oil having a Specific Gravity between 0.90 and 0.95.

Another embodiment comprises cyclosporin A, Indulin SAL®, and an oil having a Specific Gravity between 0.90 and 0.95.

Another embodiment comprises cyclosporin A, Indulin W-1®, and an oil having a Specific Gravity between 0.90 and 0.95.

Another embodiment comprises cyclosporin A, Indulin W-5®, and an oil having a Specific Gravity between 0.90 and 0.95.

Another embodiment comprises cyclosporin A, Lignosol FTA®, and an oil having a Specific Gravity between 0.90 and 0.95.

Another embodiment comprises cyclosporin A, Lignosol SFX-65®, and an oil having a Specific Gravity between 0.90 and 0.95.

Another embodiment comprises cyclosporin A, Marasperse 52 CP®, and an oil having a Specific Gravity between 0.90 and 0.95.

Another embodiment comprises cyclosporin A, Marasperse AG®, and an oil having a Specific Gravity between 0.90 and 0.95.

Another embodiment comprises cyclosporin A, Marasperse CBOS-4®, and an oil having a Specific Gravity between 0.90 and 0.95.

Another embodiment comprises cyclosporin A, Ufoxane 2®, and an oil having a Specific Gravity between 0.90 and 0.95.

Another embodiment comprises cyclosporin A, a Methyl Ester, and an oil having a Specific Gravity between 0.90 and 0.95.

Another embodiment comprises cyclosporin A, E.B. Cleaner AK®, and an oil having a Specific Gravity between 0.90 and 0.95.

Another embodiment comprises cyclosporin A, Oleocal ME-70®, and an oil having a Specific Gravity between 0.90 and 0.95.

Another embodiment comprises cyclosporin A, Trade Name Company®, and an oil having a Specific Gravity between 0.90 and 0.95.

Another embodiment comprises cyclosporin A, Methyl Esters (cont'd)®, and an oil having a Specific Gravity between 0.90 and 0.95.

Another embodiment comprises cyclosporin A, Oleocal ME-92®, and an oil having a Specific Gravity between 0.90 and 0.95.

Another embodiment comprises cyclosporin A, Oleocal ME-112®, and an oil having a Specific Gravity between 0.90 and 0.95.

Another embodiment comprises cyclosporin A, Oleocal ME-130®, and an oil having a Specific Gravity between 0.90 and 0.95.

Another embodiment comprises cyclosporin A, a Monoglyceride or a Derivative thereof, and an oil having a Specific Gravity between 0.90 and 0.95.

Another embodiment comprises cyclosporin A, Dynacet 211®, and an oil having a Specific Gravity between 0.90 and 0.95.

Another embodiment comprises cyclosporin A, Hetsorb S-20®, and an oil having a Specific Gravity between 0.90 and 0.95.

Another embodiment comprises cyclosporin A, Imwitor 191®, and an oil having a Specific Gravity between 0.90 and 0.95.

Another embodiment comprises cyclosporin A, Imwitor 370®, and an oil having a Specific Gravity between 0.90 and 0.95.

Another embodiment comprises cyclosporin A, Imwitor 375®, and an oil having a Specific Gravity between 0.90 and 0.95.

Another embodiment comprises cyclosporin A, Imwitor 900®, and an oil having a Specific Gravity between 0.90 and 0.95.

Another embodiment comprises cyclosporin A, Imwitor 945®, and an oil having a Specific Gravity between 0.90 and 0.95.

Another embodiment comprises cyclosporin A, Imwitor 2020®, and an oil having a Specific Gravity between 0.90 and 0.95.

Another embodiment comprises cyclosporin A, Kemester 5500®, and an oil having a Specific Gravity between 0.90 and 0.95.

Another embodiment comprises cyclosporin A, Kemester 6000®, and an oil having a Specific Gravity between 0.90 and 0.95.

Another embodiment comprises cyclosporin A, Magrabar GMC®, and an oil having a Specific Gravity between 0.90 and 0.95.

Another embodiment comprises cyclosporin A, Magrabar GMO-CK®, and an oil having a Specific Gravity between 0.90 and 0.95.

Another embodiment comprises cyclosporin A, Magrabar GPC-10®, and an oil having a Specific Gravity between 0.90 and 0.95.

Another embodiment comprises cyclosporin A, Magrabar MDG-5050®, and an oil having a Specific Gravity between 0.90 and 0.95.

Another embodiment comprises cyclosporin A, Monalube 335®, and an oil having a Specific Gravity between 0.90 and 0.95.

Another embodiment comprises cyclosporin A, Monoglycerides Glyceryl Monestearate Archer Daniels Midland Company®, and an oil having a Specific Gravity between 0.90 and 0.95.

Another embodiment comprises cyclosporin A, Rita GMS®, and an oil having a Specific Gravity between 0.90 and 0.95.

Another embodiment comprises cyclosporin A, Ritamulse SCG®, and an oil having a Specific Gravity between 0.90 and 0.95.

Another embodiment comprises cyclosporin A, Softigen 701®, and an oil having a Specific Gravity between 0.90 and 0.95.

Another embodiment comprises cyclosporin A, Tally 100 Plus®, and an oil having a Specific Gravity between 0.90 and 0.95.

Another embodiment comprises cyclosporin A, a Polyethylene Glycol, and an oil having a Specific Gravity between 0.90 and 0.95.

Another embodiment comprises cyclosporin A, Emulgade PL 68/50®, and an oil having a Specific Gravity between 0.90 and 0.95.

Another embodiment comprises cyclosporin A, Lumulse PEG®, and an oil having a Specific Gravity between 0.90 and 0.95.

Another embodiment comprises cyclosporin A, Rhodasurf PEG-400®, and an oil having a Specific Gravity between 0.90 and 0.95.

Another embodiment comprises cyclosporin A, Rhodasurf PEG-600®, and an oil having a Specific Gravity between 0.90 and 0.95.

Another embodiment comprises cyclosporin A, Witconol PEG-400®, and an oil having a Specific Gravity between 0.90 and 0.95.

Another embodiment comprises cyclosporin A, a Polymeric Surfactant, and an oil having a Specific Gravity between 0.90 and 0.95.

Another embodiment comprises cyclosporin A, Acritamer PNC-EG®, and an oil having a Specific Gravity between 0.90 and 0.95.

Another embodiment comprises cyclosporin A, Ag-Rho DEP-775®, and an oil having a Specific Gravity between 0.90 and 0.95.

Another embodiment comprises cyclosporin A, APG 325N Glycoside®, and an oil having a Specific Gravity between 0.90 and 0.95.

Another embodiment comprises cyclosporin A, Aristoflex AVC®, and an oil having a Specific Gravity between 0.90 and 0.95.

Another embodiment comprises cyclosporin A, Aristoflex HMB®, and an oil having a Specific Gravity between 0.90 and 0.95.

Another embodiment comprises cyclosporin A, Burco NPS-225®, and an oil having a Specific Gravity between 0.90 and 0.95.

Another embodiment comprises cyclosporin A, Burco NPS-816®, and an oil having a Specific Gravity between 0.90 and 0.95.

Another embodiment comprises cyclosporin A, Chemccinate 5603®, and an oil having a Specific Gravity between 0.90 and 0.95.

Another embodiment comprises cyclosporin A, Cosmedia Guar C-261N®, and an oil having a Specific Gravity between 0.90 and 0.95.

Another embodiment comprises cyclosporin A, Gantrez S-95®, and an oil having a Specific Gravity between 0.90 and 0.95.

Another embodiment comprises cyclosporin A, Glucopon 220 UP®, and an oil having a Specific Gravity between 0.90 and 0.95.

Another embodiment comprises cyclosporin A, Glucopon 225 DK®, and an oil having a Specific Gravity between 0.90 and 0.95.

Another embodiment comprises cyclosporin A, Glucopon 425 N®, and an oil having a Specific Gravity between 0.90 and 0.95.

Another embodiment comprises cyclosporin A, Glucopon 600 UP®, and an oil having a Specific Gravity between 0.90 and 0.95.

Another embodiment comprises cyclosporin A, Glucopon 625 UP®, and an oil having a Specific Gravity between 0.90 and 0.95.

Another embodiment comprises cyclosporin A, Pemulen 1621®, and an oil having a Specific Gravity between 0.90 and 0.95.

Another embodiment comprises cyclosporin A, Pemulen 1622®, and an oil having a Specific Gravity between 0.90 and 0.95.

Another embodiment comprises cyclosporin A, Pemulen TR-1®, and an oil having a Specific Gravity between 0.90 and 0.95.

Another embodiment comprises cyclosporin A, Pemulen TR-2®, and an oil having a Specific Gravity between 0.90 and 0.95.

Another embodiment comprises cyclosporin A, Plantacare 818®, and an oil having a Specific Gravity between 0.90 and 0.95.

Another embodiment comprises cyclosporin A, Plantapon LGC Sorb®, and an oil having a Specific Gravity between 0.90 and 0.95.

Another embodiment comprises cyclosporin A, Plantaren 1200N®, and an oil having a Specific Gravity between 0.90 and 0.95.

Another embodiment comprises cyclosporin A, Plantaren 2000N®, and an oil having a Specific Gravity between 0.90 and 0.95.

Another embodiment comprises cyclosporin A, Viscolam AT 64®, and an oil having a Specific Gravity between 0.90 and 0.95.

Another embodiment comprises cyclosporin A, Viscolam AT 64P®, and an oil having a Specific Gravity between 0.90 and 0.95.

Another embodiment comprises cyclosporin A, Viscolam AT 100®, and an oil having a Specific Gravity between 0.90 and 0.95.

Another embodiment comprises cyclosporin A, Viscolam MAC 7®, and an oil having a Specific Gravity between 0.90 and 0.95.

Another embodiment comprises cyclosporin A, Viscolam SMC 20®, and an oil having a Specific Gravity between 0.90 and 0.95.

Another embodiment comprises cyclosporin A, Witbreak RTC-323®, and an oil having a Specific Gravity between 0.90 and 0.95.

Another embodiment comprises cyclosporin A, WSI 3700®, and an oil having a Specific Gravity between 0.90 and 0.95.

Another embodiment comprises cyclosporin A, a Propoxylated or Ethoxylated Fatty Acid, Alcohol, or Alkyl Phenol, and an oil having a Specific Gravity between 0.90 and 0.95.

Another embodiment comprises cyclosporin A, Antarox AA-60®, and an oil having a Specific Gravity between 0.90 and 0.95.

Another embodiment comprises cyclosporin A, Antarox LF-224®, and an oil having a Specific Gravity between 0.90 and 0.95.

Another embodiment comprises cyclosporin A, Burcomul DFE-45®, and an oil having a Specific Gravity between 0.90 and 0.95.

Another embodiment comprises cyclosporin A, Burcoterge LFE-1000®, and an oil having a Specific Gravity between 0.90 and 0.95.

Another embodiment comprises cyclosporin A, Chemal LF-25B®, and an oil having a Specific Gravity between 0.90 and 0.95.

Another embodiment comprises cyclosporin A, Chemal LF-40B®, and an oil having a Specific Gravity between 0.90 and 0.95.

Another embodiment comprises cyclosporin A, Dehypon LS-36®, and an oil having a Specific Gravity between 0.90 and 0.95.

Another embodiment comprises cyclosporin A, Dehypon LS-36®, and an oil having a Specific Gravity between 0.90 and 0.95.

Another embodiment comprises cyclosporin A, Dehypon LS-54®, and an oil having a Specific Gravity between 0.90 and 0.95.

Another embodiment comprises cyclosporin A, Dehypon LS-54®, and an oil having a Specific Gravity between 0.90 and 0.95.

Another embodiment comprises cyclosporin A, Delonic 100 VLF®, and an oil having a Specific Gravity between 0.90 and 0.95.

Another embodiment comprises cyclosporin A, Delonic LF-60 MOD®, and an oil having a Specific Gravity between 0.90 and 0.95.

Another embodiment comprises cyclosporin A, Empiderm B®, and an oil having a Specific Gravity between 0.90 and 0.95.

Another embodiment comprises cyclosporin A, Ethylan 1206®, and an oil having a Specific Gravity between 0.90 and 0.95.

Another embodiment comprises cyclosporin A, Ethylan NS-500K®, and an oil having a Specific Gravity between 0.90 and 0.95.

Another embodiment comprises cyclosporin A, Ethylan NS-500LQ®, and an oil having a Specific Gravity between 0.90 and 0.95.

Another embodiment comprises cyclosporin A, Genapol 1392®, and an oil having a Specific Gravity between 0.90 and 0.95.

Another embodiment comprises cyclosporin A, Genapol 2317®, and an oil having a Specific Gravity between 0.90 and 0.95.

Another embodiment comprises cyclosporin A, Genapol 26EP710®, and an oil having a Specific Gravity between 0.90 and 0.95.

Another embodiment comprises cyclosporin A, Genapol EP 1022®, and an oil having a Specific Gravity between 0.90 and 0.95.

Another embodiment comprises cyclosporin A, Genapol EP 1024®, and an oil having a Specific Gravity between 0.90 and 0.95.

Another embodiment comprises cyclosporin A, Genapol EP 6068®, and an oil having a Specific Gravity between 0.90 and 0.95.

Another embodiment comprises cyclosporin A, Genapol NP915®, and an oil having a Specific Gravity between 0.90 and 0.95.

Another embodiment comprises cyclosporin A, Kieralon MFB®, and an oil having a Specific Gravity between 0.90 and 0.95.

Another embodiment comprises cyclosporin A, Lumisolve CSA-80 V®, and an oil having a Specific Gravity between 0.90 and 0.95.

Another embodiment comprises cyclosporin A, Marlowet 5001®, and an oil having a Specific Gravity between 0.90 and 0.95.

Another embodiment comprises cyclosporin A, Marlox FK 64®, and an oil having a Specific Gravity between 0.90 and 0.95.

Another embodiment comprises cyclosporin A, Marlox MO 124®, and an oil having a Specific Gravity between 0.90 and 0.95.

Another embodiment comprises cyclosporin A, Marlox S 58®, and an oil having a Specific Gravity between 0.90 and 0.95.

Another embodiment comprises cyclosporin A, Nonatell 1003®, and an oil having a Specific Gravity between 0.90 and 0.95.

Another embodiment comprises cyclosporin A, Nonatell 1038®, and an oil having a Specific Gravity between 0.90 and 0.95.

Another embodiment comprises cyclosporin A, Nonatell 1052®, and an oil having a Specific Gravity between 0.90 and 0.95.

Another embodiment comprises cyclosporin A, Nonatell 1061®, and an oil having a Specific Gravity between 0.90 and 0.95.

Another embodiment comprises cyclosporin A, Nonatell 1075®, and an oil having a Specific Gravity between 0.90 and 0.95.

Another embodiment comprises cyclosporin A, Nonatell 1088®, and an oil having a Specific Gravity between 0.90 and 0.95.

Another embodiment comprises cyclosporin A, Nonatell 1123®, and an oil having a Specific Gravity between 0.90 and 0.95.

Another embodiment comprises cyclosporin A, Nonatell 1153®, and an oil having a Specific Gravity between 0.90 and 0.95.

Another embodiment comprises cyclosporin A, Nonatell 1161®, and an oil having a Specific Gravity between 0.90 and 0.95.

Another embodiment comprises cyclosporin A, Nonatell 1172®, and an oil having a Specific Gravity between 0.90 and 0.95.

Another embodiment comprises cyclosporin A, Nonatell 1181®, and an oil having a Specific Gravity between 0.90 and 0.95.

Another embodiment comprises cyclosporin A, Norfox 36®, and an oil having a Specific Gravity between 0.90 and 0.95.

Another embodiment comprises cyclosporin A, Procetyl AWS®, and an oil having a Specific Gravity between 0.90 and 0.95.

Another embodiment comprises cyclosporin A, Sandoxylate SX 412®, and an oil having a Specific Gravity between 0.90 and 0.95.

Another embodiment comprises cyclosporin A, Sandoxylate SX 418®, and an oil having a Specific Gravity between 0.90 and 0.95.

Another embodiment comprises cyclosporin A, Surfonic JL-80X®, and an oil having a Specific Gravity between 0.90 and 0.95.

Another embodiment comprises cyclosporin A, Sunfonic JL-80X-B1®, and an oil having a Specific Gravity between 0.90 and 0.95.

Another embodiment comprises cyclosporin A, Sunfonic L4-29X®, and an oil having a Specific Gravity between 0.90 and 0.95.

Another embodiment comprises cyclosporin A, Surfonic LF®, and an oil having a Specific Gravity between 0.90 and 0.95.

Another embodiment comprises cyclosporin A, T-Det A826®, and an oil having a Specific Gravity between 0.90 and 0.95.

Another embodiment comprises cyclosporin A, T-Det LF-416®, and an oil having a Specific Gravity between 0.90 and 0.95.

Another embodiment comprises cyclosporin A, Tergitol Min-Foam 1X®, and an oil having a Specific Gravity between 0.90 and 0.95.

Another embodiment comprises cyclosporin A, Tergitol Min-Foam 2X®, and an oil having a Specific Gravity between 0.90 and 0.95.

Another embodiment comprises cyclosporin A, Triton CF-21®, and an oil having a Specific Gravity between 0.90 and 0.95.

Another embodiment comprises cyclosporin A, Triton CF-76®, and an oil having a Specific Gravity between 0.90 and 0.95.

Another embodiment comprises cyclosporin A, Triton XL-80N®, and an oil having a Specific Gravity between 0.90 and 0.95.

Another embodiment comprises cyclosporin A, Witconol NS-98®, and an oil having a Specific Gravity between 0.90 and 0.95.

Another embodiment comprises cyclosporin A, Witconol NS-108LQ®, and an oil having a Specific Gravity between 0.90 and 0.95.

Another embodiment comprises cyclosporin A, Witconol NS-145®, and an oil having a Specific Gravity between 0.90 and 0.95.

Another embodiment comprises cyclosporin A, Witconol NS-179®, and an oil having a Specific Gravity between 0.90 and 0.95.

Another embodiment comprises cyclosporin A, a Protein-based Surfactant, and an oil having a Specific Gravity between 0.90 and 0.95.

Another embodiment comprises cyclosporin A, AminoFoam W®, and an oil having a Specific Gravity between 0.90 and 0.95.

Another embodiment comprises cyclosporin A, Amiter LGOD-2®, and an oil having a Specific Gravity between 0.90 and 0.95.

Another embodiment comprises cyclosporin A, Amiter LGS-2®, and an oil having a Specific Gravity between 0.90 and 0.95.

Another embodiment comprises cyclosporin A, Amiter LGS-5®, and an oil having a Specific Gravity between 0.90 and 0.95.

Another embodiment comprises cyclosporin A, Lamepon S®, and an oil having a Specific Gravity between 0.90 and 0.95.

Another embodiment comprises cyclosporin A, Lamepon S®, and an oil having a Specific Gravity between 0.90 and 0.95.

Another embodiment comprises cyclosporin A, Maypon 4C®, and an oil having a Specific Gravity between 0.90 and 0.95.

Another embodiment comprises cyclosporin A, May-Tein C®, and an oil having a Specific Gravity between 0.90 and 0.95.

Another embodiment comprises cyclosporin A, May-Tein CT®, and an oil having a Specific Gravity between 0.90 and 0.95.

Another embodiment comprises cyclosporin A, May-Tein KTS®, and an oil having a Specific Gravity between 0.90 and 0.95.

Another embodiment comprises cyclosporin A, May-Tein SY®, and an oil having a Specific Gravity between 0.90 and 0.95.

Another embodiment comprises cyclosporin A, Plantapon S®, and an oil having a Specific Gravity between 0.90 and 0.95.

Another embodiment comprises cyclosporin A, Proteol APL®, and an oil having a Specific Gravity between 0.90 and 0.95.

Another embodiment comprises cyclosporin A, Proteol OAT®, and an oil having a Specific Gravity between 0.90 and 0.95.

Another embodiment comprises cyclosporin A, Pyroter CPI-40®, and an oil having a Specific Gravity between 0.90 and 0.95.

Another embodiment comprises cyclosporin A, Pyroter GPI-25®, and an oil having a Specific Gravity between 0.90 and 0.95.

Another embodiment comprises cyclosporin A, Supro-Tein S®, and an oil having a Specific Gravity between 0.90 and 0.95.

Another embodiment comprises cyclosporin A, Supro-Tein V®, and an oil having a Specific Gravity between 0.90 and 0.95.

Another embodiment comprises cyclosporin A, a Sarcosine Derivative®, and an oil having a Specific Gravity between 0.90 and 0.95.

Another embodiment comprises cyclosporin A, Crodasinic LS-30®, and an oil having a Specific Gravity between 0.90 and 0.95.

Another embodiment comprises cyclosporin A, Vanseal CS®, and an oil having a Specific Gravity between 0.90 and 0.95.

Another embodiment comprises cyclosporin A, Vanseal LS®, and an oil having a Specific Gravity between 0.90 and 0.95.

Another embodiment comprises cyclosporin A, Vanseal MS®, and an oil having a Specific Gravity between 0.90 and 0.95.

Another embodiment comprises cyclosporin A, Vanseal NACS-30®, and an oil having a Specific Gravity between 0.90 and 0.95.

Another embodiment comprises cyclosporin A, Vanseal NALS-95®, and an oil having a Specific Gravity between 0.90 and 0.95.

Another embodiment comprises cyclosporin A, Vanseal OS®, and an oil having a Specific Gravity between 0.90 and 0.95.

Another embodiment comprises cyclosporin A, a Silicone-based Surfactant®, and an oil having a Specific Gravity between 0.90 and 0.95.

Another embodiment comprises cyclosporin A, Abil-B-9950®, and an oil having a Specific Gravity between 0.90 and 0.95.

Another embodiment comprises cyclosporin A, Abil Care 85®, and an oil having a Specific Gravity between 0.90 and 0.95.

Another embodiment comprises cyclosporin A, Abil EM 90®, and an oil having a Specific Gravity between 0.90 and 0.95.

Another embodiment comprises cyclosporin A, Abil EM 97®, and an oil having a Specific Gravity between 0.90 and 0.95.

Another embodiment comprises cyclosporin A, Abil WE-09®, and an oil having a Specific Gravity between 0.90 and 0.95.

Another embodiment comprises cyclosporin A, Dow Corning 1248 Fluid®, and an oil having a Specific Gravity between 0.90 and 0.95.

Another embodiment comprises cyclosporin A, Dow Corning 3225C®, and an oil having a Specific Gravity between 0.90 and 0.95.

Another embodiment comprises cyclosporin A, Dow Corning 5200®, and an oil having a Specific Gravity between 0.90 and 0.95.

Another embodiment comprises cyclosporin A, Dow Corning Q4-3667®, and an oil having a Specific Gravity between 0.90 and 0.95.

Another embodiment comprises cyclosporin A, Monasil PCA®, and an oil having a Specific Gravity between 0.90 and 0.95.

Another embodiment comprises cyclosporin A, Monasil PDM®, and an oil having a Specific Gravity between 0.90 and 0.95.

Another embodiment comprises cyclosporin A, Monasil PLN®, and an oil having a Specific Gravity between 0.90 and 0.95.

Another embodiment comprises cyclosporin A, Polyderm PPI-SI-WS®, and an oil having a Specific Gravity between 0.90 and 0.95.

Another embodiment comprises cyclosporin A, Troysol 380W®, and an oil having a Specific Gravity between 0.90 and 0.95.

Another embodiment comprises cyclosporin A, Troysol S366®, and an oil having a Specific Gravity between 0.90 and 0.95.

Another embodiment comprises cyclosporin A, a Sorbitan Derivative, and an oil having a Specific Gravity between 0.90 and 0.95.

Another embodiment comprises cyclosporin A, Alkamuls SML®, and an oil having a Specific Gravity between 0.90 and 0.95.

Another embodiment comprises cyclosporin A, Alkamuls SMO®, and an oil having a Specific Gravity between 0.90 and 0.95.

Another embodiment comprises cyclosporin A, Alkamuls STO®, and an oil having a Specific Gravity between 0.90 and 0.95.

Another embodiment comprises cyclosporin A, Arlacel 20®, and an oil having a Specific Gravity between 0.90 and 0.95.

Another embodiment comprises cyclosporin A, Arlacel 40®, and an oil having a Specific Gravity between 0.90 and 0.95.

Another embodiment comprises cyclosporin A, Arlacel 60®, and an oil having a Specific Gravity between 0.90 and 0.95.

Another embodiment comprises cyclosporin A, Arlacel 80®, and an oil having a Specific Gravity between 0.90 and 0.95.

Another embodiment comprises cyclosporin A, Arlacel C®, and an oil having a Specific Gravity between 0.90 and 0.95.

Another embodiment comprises cyclosporin A, Armul 21®, and an oil having a Specific Gravity between 0.90 and 0.95.

Another embodiment comprises cyclosporin A, Atlox 80®, and an oil having a Specific Gravity between 0.90 and 0.95.

Another embodiment comprises cyclosporin A, Atlox 847®, and an oil having a Specific Gravity between 0.90 and 0.95.

Another embodiment comprises cyclosporin A, Atlox 1045A®, and an oil having a Specific Gravity between 0.90 and 0.95.

Another embodiment comprises cyclosporin A, Canarcel 20®, and an oil having-a Specific Gravity between 0.90 and 0.95.

Another embodiment comprises cyclosporin A, Canarcel 60®, and an oil having a Specific Gravity between 0.90 and 0.95.

Another embodiment comprises cyclosporin A, Canarcel 80®, and an oil having a Specific Gravity between 0.90 and 0.95.

Another embodiment comprises cyclosporin A, Canarcel TW 20®, and an oil having a Specific Gravity between 0.90 and 0.95.

Another embodiment comprises cyclosporin A, Canarcel TW 60®, and an oil having a Specific Gravity between 0.90 and 0.95.

Another embodiment comprises cyclosporin A, Canarcel TW 80®, and an oil having a Specific Gravity between 0.90 and 0.95.

Another embodiment comprises cyclosporin A, Coladet BSB-P®, and an oil having a Specific Gravity between 0.90 and 0.95.

Another embodiment comprises cyclosporin A, Customulse O-20®, and an oil having a Specific Gravity between 0.90 and 0.95.

Another embodiment comprises cyclosporin A, Dehymuls E®, and an oil having a Specific Gravity between 0.90 and 0.95.

Another embodiment comprises cyclosporin A, DeSotan SMO®, and an oil having a Specific Gravity between 0.90 and 0.95.

Another embodiment comprises cyclosporin A, DeSotan SMO-20®, and an oil having a Specific Gravity between 0.90 and 0.95.

Another embodiment comprises cyclosporin A, DeSotan SMT®, and an oil having a Specific Gravity between 0.90 and 0.95.

Another embodiment comprises cyclosporin A, DeSotan SMT-20®, and an oil having a Specific Gravity between 0.90 and 0.95.

Another embodiment comprises cyclosporin A, Durfax 60®, and an oil having a Specific Gravity between 0.90 and 0.95.

Another embodiment comprises cyclosporin A, Durfax 65®, and an oil having a Specific Gravity between 0.90 and 0.95.

Another embodiment comprises cyclosporin A, Durfax 80®, and an oil having a Specific Gravity between 0.90 and 0.95.

Another embodiment comprises cyclosporin A, Durtan 60®, and an oil having a Specific Gravity between 0.90 and 0.95.

Another embodiment comprises cyclosporin A, Durtan 65®, and an oil having a Specific Gravity between 0.90 and 0.95.

Another embodiment comprises cyclosporin A, Liposorb L®, and an oil having a Specific Gravity between 0.90 and 0.95.

Another embodiment comprises cyclosporin A, Liposorb L-10®, and an oil having a Specific Gravity between 0.90 and 0.95.

Another embodiment comprises cyclosporin A, Liposorb L-20®, and an oil having a Specific Gravity between 0.90 and 0.95.

Another embodiment comprises cyclosporin A, Liposorb O®, and an oil having a Specific Gravity between 0.90 and 0.95.

Another embodiment comprises cyclosporin A, Liposorb O-20®, and an oil having a Specific Gravity between 0.90 and 0.95.

Another embodiment comprises cyclosporin A, Liposorb P®, and an oil having a Specific Gravity between 0.90 and 0.95.

Another embodiment comprises cyclosporin A, Liposorb P-20®, and an oil having a Specific Gravity between 0.90 and 0.95.

Another embodiment comprises cyclosporin A, Liposorb S®, and an oil having a Specific Gravity between 0.90 and 0.95.

Another embodiment comprises cyclosporin A, Liposorb S-20®, and an oil having a Specific Gravity between 0.90 and 0.95.

Another embodiment comprises cyclosporin A, Liposorb SQO®, and an oil having a Specific Gravity between 0.90 and 0.95.

Another embodiment comprises cyclosporin A, Liposorb TO®, and an oil having a Specific Gravity between 0.90 and 0.95.

Another embodiment comprises cyclosporin A, Liposorb TS®, and an oil having a Specific Gravity between 0.90 and 0.95.

Another embodiment comprises cyclosporin A, Liposorb TS-20®, and an oil having a Specific Gravity between 0.90 and 0.95.

Another embodiment comprises cyclosporin A, Lumisorb PS®, and an oil having a Specific Gravity between 0.90 and 0.95.

Another embodiment comprises cyclosporin A, Lumisorb SMO (T)®, and an oil having a Specific Gravity between 0.90 and 0.95.

Another embodiment comprises cyclosporin A, Lumisorb SMS K®, and an oil having a Specific Gravity between 0.90 and 0.95.

Another embodiment comprises cyclosporin A, Lumisorb SSO®, and an oil having a Specific Gravity between 0.90 and 0.95.

Another embodiment comprises cyclosporin A, Lumisorb STS K®, and an oil having a Specific Gravity between 0.90 and 0.95.

Another embodiment comprises cyclosporin A, Lumisorb STT®, and an oil having a Specific Gravity between 0.90 and 0.95.

Another embodiment comprises cyclosporin A, Magrabar SMO®, and an oil having a Specific Gravity between 0.90 and 0.95.

Another embodiment comprises cyclosporin A, Magrabar SMO-VEG®, and an oil having a Specific Gravity between 0.90 and 0.95.

Another embodiment comprises cyclosporin A, Magrabar SMT®, and an oil having a Specific Gravity between 0.90 and 0.95.

Another embodiment comprises cyclosporin A, Magrabar STO®, and an oil having a Specific Gravity between 0.90 and 0.95.

Another embodiment comprises cyclosporin A, Miracare BC-27®, and an oil having a Specific Gravity between 0.90 and 0.95.

Another embodiment comprises cyclosporin A, Ritabate 20®, and an oil having a Specific Gravity between 0.90 and 0.95.

Another embodiment comprises cyclosporin A, Ritabate 40®, and an oil having a Specific Gravity between 0.90 and 0.95.

Another embodiment comprises cyclosporin A, Ritabate 60®, and an oil having a Specific Gravity between 0.90 and 0.95.

Another embodiment comprises cyclosporin A, Ritabate 80®, and an oil having a Specific Gravity between 0.90 and 0.95.

Another embodiment comprises cyclosporin A, T-Maz®, and an oil having a Specific Gravity between 0.90 and 0.95.

Another embodiment comprises cyclosporin A, Tego SML®, and an oil having a Specific Gravity between 0.90 and 0.95.

Another embodiment comprises cyclosporin A, Tego SML 20®, and an oil having a Specific Gravity between 0.90 and 0.95.

Another embodiment comprises cyclosporin A, Tego SMO 80 V®, and an oil having a Specific Gravity between 0.90 and 0.95.

Another embodiment comprises cyclosporin A, Tego SMO V®, and an oil having a Specific Gravity between 0.90 and 0.95.

Another embodiment comprises cyclosporin A, Tego SMS®, and an oil having a Specific Gravity between 0.90 and 0.95.

Another embodiment comprises cyclosporin A, Tego STO V®, and an oil having a Specific Gravity between 0.90 and 0.95.

Another embodiment comprises cyclosporin A, Tween 21®, and an oil having a Specific Gravity between 0.90 and 0.95.

Another embodiment comprises cyclosporin A, Tween 40®, and an oil having a Specific Gravity between 0.90 and 0.95.

Another embodiment comprises cyclosporin A, Tween 60®, and an oil having a Specific Gravity between 0.90 and 0.95.

Another embodiment comprises cyclosporin A, Tween 60 K®, and an oil having a Specific Gravity between 0.90 and 0.95.

Another embodiment comprises cyclosporin A, Tween 61®, and an oil having a Specific Gravity between 0.90 and 0.95.

Another embodiment comprises cyclosporin A, Tween 65®, and an oil having a Specific Gravity between 0.90 and 0.95.

Another embodiment comprises cyclosporin A, Tween 80®, and an oil having a Specific Gravity between 0.90 and 0.95.

Another embodiment comprises cyclosporin A, Tween 80 K®, and an oil having a Specific Gravity between 0.90 and 0.95.

Another embodiment comprises cyclosporin A, Tween 81®, and an oil having a Specific Gravity between 0.90 and 0.95.

Another embodiment comprises cyclosporin A, Tween 85®, and an oil having a Specific Gravity between 0.90 and 0.95.

Another embodiment comprises cyclosporin A, a Sucrose or Glucose Ester, or Derivative thereof, and an oil having a Specific Gravity between 0.90 and 0.95.

Another embodiment comprises cyclosporin A, DeSulf GOS-P-60WCG®, and an oil having a Specific Gravity between 0.90 and 0.95.

Another embodiment comprises cyclosporin A, Glucam E-20 Distearate®, and an oil having a Specific Gravity between 0.90 and 0.95.

Another embodiment comprises cyclosporin A, Glucamate DOE-120®, and an oil having a Specific Gravity between 0.90 and 0.95.

Another embodiment comprises cyclosporin A, Glucamate SSE-20®, and an oil having a Specific Gravity between 0.90 and 0.95.

Another embodiment comprises cyclosporin A, Glucate DO®, and an oil having a Specific Gravity between 0.90 and 0.95.

Another embodiment comprises cyclosporin A, Glucate SS®, and an oil having a Specific Gravity between 0.90 and 0.95.

Another embodiment comprises cyclosporin A, Glucopon 425 UP®, and an oil having a Specific Gravity between 0.90 and 0.95.

Another embodiment comprises cyclosporin A, Isolan IS®, and an oil having a Specific Gravity between 0.90 and 0.95.

Another embodiment comprises cyclosporin A, Mazon 40®, and an oil having a Specific Gravity between 0.90 and 0.95.

Another embodiment comprises cyclosporin A, Montanov 82®, and an oil having a Specific Gravity between 0.90 and 0.95.

Another embodiment comprises cyclosporin A, Montanov 202®, and an oil having a Specific Gravity between 0.90 and 0.95.

Another embodiment comprises cyclosporin A, Montanov S®, and an oil having a Specific Gravity between 0.90 and 0.95.

Another embodiment comprises cyclosporin A, Rheozan®, and an oil having a Specific Gravity between 0.90 and 0.95.

Another embodiment comprises cyclosporin A, Simulsol AS 48®, and an oil having a Specific Gravity between 0.90 and 0.95.

Another embodiment comprises cyclosporin A, Simulsol SL 4®, and an oil having a Specific Gravity between 0.90 and 0.95.

Another embodiment comprises cyclosporin A, Simulsol SL 10®, and an oil having a Specific Gravity between 0.90 and 0.95.

Another embodiment comprises cyclosporin A, Simulsol SL 11W®, and an oil having a Specific Gravity between 0.90 and 0.95.

Another embodiment comprises cyclosporin A, Simulsol SL 55®, and an oil having a Specific Gravity between 0.90 and 0.95.

Another embodiment comprises cyclosporin A, Suga Nate 100 and 160®, and an oil having a Specific Gravity between 0.90 and 0.95.

Another embodiment comprises cyclosporin A, Tego Care 450®, and an oil having a Specific Gravity between 0.90 and 0.95.

Another embodiment comprises cyclosporin A, Tego Care CG 90®, and an oil having a Specific Gravity between 0.90 and 0.95.

Another embodiment comprises cyclosporin A, Tego Care PS®, and an oil having a Specific Gravity between 0.90 and 0.95.

Another embodiment comprises cyclosporin A, Tegosoft PSE 141 G®, and an oil having a Specific Gravity between 0.90 and 0.95.

Another embodiment comprises cyclosporin A, Tegotens G 826®, and an oil having a Specific Gravity between 0.90 and 0.95.

Another embodiment comprises cyclosporin A, Triton BG-10 (70%)®, and an oil having a Specific Gravity between 0.90 and 0.95.

Another embodiment comprises cyclosporin A, Triton CG-110 (60%)®, and an oil having a Specific Gravity between 0.90 and 0.95.

Another embodiment comprises cyclosporin A, Wickenol 545®, and an oil having a Specific Gravity between 0.90 and 0.95.

Another embodiment comprises cyclosporin A, an alcohol, and Almond oil.

Another embodiment comprises cyclosporin A, Diglycerol®, and Almond oil.

Another embodiment comprises cyclosporin A, Hetoxide GT-80®, and Almond oil.

Another embodiment comprises cyclosporin A, Lexemul BEO®, and Almond oil.

Another embodiment comprises cyclosporin A, Polyglycerol-3®, and Almond oil.

Another embodiment comprises cyclosporin A, Redicote E Series®, and Almond oil.

Another embodiment comprises cyclosporin A, Simulsol OX 1005L®, and Almond oil.

Another embodiment comprises cyclosporin A, Stanfax 567®, and Almond oil.

Another embodiment comprises cyclosporin A, TA-1618®, and Almond oil.

Another embodiment comprises cyclosporin A, Witconol H-31 A®, and Almond oil.

Another embodiment comprises cyclosporin A, an amine oxide, and Almond oil.

Another embodiment comprises cyclosporin A, AO-405®, and Almond oil.

Another embodiment comprises cyclosporin A, AO-455®, and Almond oil.

Another embodiment comprises cyclosporin A, AO 728 Special®, and Almond oil.

Another embodiment comprises cyclosporin A, Barlox 12®, and Almond oil.

Another embodiment comprises cyclosporin A, Barlox 14®, and Almond oil.

Another embodiment comprises cyclosporin A, Burcoxide Lo®, and Almond oil.

Another embodiment comprises cyclosporin A, Caloxamine LO®, and Almond oil.

Another embodiment comprises cyclosporin A, Chemoxide CAW®, and Almond oil.

Another embodiment comprises cyclosporin A, Chemoxide LM-30®, and Almond oil.

Another embodiment comprises cyclosporin A, Chemoxide LO®, and Almond oil.

Another embodiment comprises cyclosporin A, Chemoxide MO®, and Almond oil.

Another embodiment comprises cyclosporin A, Colalux CAO-35®, and Almond oil.

Another embodiment comprises cyclosporin A, Colalux LO®, and Almond oil.

Another embodiment comprises cyclosporin A, DeMox CAPO®, and Almond oil.

Another embodiment comprises cyclosporin A, DeMox CSG-30®, and Almond oil.

Another embodiment comprises cyclosporin A, DeMox LAO®, and Almond oil.

Another embodiment comprises cyclosporin A, Emcol L®, and Almond oil.

Another embodiment comprises cyclosporin A, Empigen OB®, and Almond oil.

Another embodiment comprises cyclosporin A, Empigen OS/A®, and Almond oil.

Another embodiment comprises cyclosporin A, Foamox CDO®, and Almond oil.

Another embodiment comprises cyclosporin A, Foamox DMM®, and Almond oil.

Another embodiment comprises cyclosporin A, Foamox DMS®, and Almond oil.

Another embodiment comprises cyclosporin A, Genaminox KC®, and Almond oil.

Another embodiment comprises cyclosporin A, Genaminox LA®, and Almond oil.

Another embodiment comprises cyclosporin A, Hartofoam SAO®, and Almond oil.

Another embodiment comprises cyclosporin A, Hartox DMCD®, and Almond oil.

Another embodiment comprises cyclosporin A, Lipowax DAT®, and Almond oil.

Another embodiment comprises cyclosporin A, Lipowax PB Pastilles®, and Almond oil.

Another embodiment comprises cyclosporin A, Mackamine C8®, and Almond oil.

Another embodiment comprises cyclosporin A, Mackamine C10®, and Almond oil.

Another embodiment comprises cyclosporin A, Mackamine C14®, and Almond oil.

Another embodiment comprises cyclosporin A, Mackamine CAO®, and Almond oil.

Another embodiment comprises cyclosporin A, Mackamine CO®, and Almond oil.

Another embodiment comprises cyclosporin A, Mackamine LO®, and Almond oil.

Another embodiment comprises cyclosporin A, Mackamine O2®, and Almond oil.

Another embodiment comprises cyclosporin A, Mackamine SAO®, and Almond oil.

Another embodiment comprises cyclosporin A, Mackamine SO®, and Almond oil.

Another embodiment comprises cyclosporin A, Mazox KCAO®, and Almond oil.

Another embodiment comprises cyclosporin A, Monalac MO®, and Almond oil.

Another embodiment comprises cyclosporin A, Nonox LDA®, and Almond oil.

Another embodiment comprises cyclosporin A, Rhodamox LO®, and Almond oil.

Another embodiment comprises cyclosporin A, Schercamox C-AA®, and Almond oil.

Another embodiment comprises cyclosporin A, Schercamox DMA®, and Almond oil.

Another embodiment comprises cyclosporin A, Schercamox DML®, and Almond oil.

Another embodiment comprises cyclosporin A, Schercamox DMM®, and Almond oil;

Another embodiment comprises cyclosporin A, Schercamox DMS®, and Almond oil.

Another embodiment comprises cyclosporin A, Tegotens DO®, and Almond oil.

Another embodiment comprises cyclosporin A, Tomah AO-14-2®, and Almond oil.

Another embodiment comprises cyclosporin A, Triaminox CDO®, and Almond oil.

Another embodiment comprises cyclosporin A, a block Polymer, and Almond oil.

Another embodiment comprises cyclosporin A, AL 2070®, and Almond oil.

Another embodiment comprises cyclosporin A, Antarox 17-R-2®, and Almond oil.

Another embodiment comprises cyclosporin A, Antarox 25-R-2®, and Almond oil.

Another embodiment comprises cyclosporin A, Antarox 31-R-1®, and Almond oil.

Another embodiment comprises cyclosporin A, Antarox P-84®, and Almond oil.

Another embodiment comprises cyclosporin A, Antarox P-104/H®, and Almond oil.

Another embodiment comprises cyclosporin A, Arnox BP-Series®, and Almond oil.

Another embodiment comprises cyclosporin A, Chemonic 435®, and Almond oil.

Another embodiment comprises cyclosporin A, Chemonic D-25®, and Almond oil.

Another embodiment comprises cyclosporin A, Chemonic PL Series®, and Almond oil.

Another embodiment comprises cyclosporin A, Ethox L-121®, and Almond oil.

Another embodiment comprises cyclosporin A, Ethox L-122®, and Almond oil.

Another embodiment comprises cyclosporin A, Genapol PF-10®, and Almond oil.

Another embodiment comprises cyclosporin A, Genapol PF-20®, and Almond oil.

Another embodiment comprises cyclosporin A, Genapol PF-40A®, and Almond oil.

Another embodiment comprises cyclosporin A, Norfox 2-LF®, and Almond oil.

Another embodiment comprises cyclosporin A, Simulsol NW 342®, and Almond oil.

Another embodiment comprises cyclosporin A, T-Det BP-1®, and Almond oil.

Another embodiment comprises cyclosporin A, T-Det XD®, and Almond oil.

Another embodiment comprises cyclosporin A, T-Det XH®, and Almond oil.

Another embodiment comprises cyclosporin A, Triton CF-32®, and Almond oil.

Another embodiment comprises cyclosporin A, Witconol 171®, and Almond oil.

Another embodiment comprises cyclosporin A, Witconol 324®, and Almond oil.

Another embodiment comprises cyclosporin A, Witconol 324D®, and Almond oil.

Another embodiment comprises cyclosporin A, Witconol PD-2000®, and Almond oil.

Another embodiment comprises cyclosporin A, a Carboxylated Alcohol or Alkylphenol Ethoxylate, and Almond oil.

Another embodiment comprises cyclosporin A, Emcol CN-6®, and Almond oil.

Another embodiment comprises cyclosporin A, Ethcarb®, and Almond oil.

Another embodiment comprises cyclosporin A, Gemtex WNT-Conc®, and Almond oil.

Another embodiment comprises cyclosporin A, Incrodet TD7-C®, and Almond oil.

Another embodiment comprises cyclosporin A, Marlinat CM 105/80®, and Almond oil.

Another embodiment comprises cyclosporin A, Marlowet 1072®, and Almond oil.

Another embodiment comprises cyclosporin A, Marlowet 4530®, and Almond oil.

Another embodiment comprises cyclosporin A, Marlowet 4530 LF®, and Almond oil.

Another embodiment comprises cyclosporin A, Marlowet 4534®, and Almond oil.

Another embodiment comprises cyclosporin A, Marlowet 4538®, and Almond oil.

Another embodiment comprises cyclosporin A, Marlowet 4539®, and Almond oil.

Another embodiment comprises cyclosporin A, Marlowet 4539 LF®, and Almond oil.

Another embodiment comprises cyclosporin A, Marlowet 4541®, and Almond oil.

Another embodiment comprises cyclosporin A, Miranate LEC-80®, and Almond oil.

Another embodiment comprises cyclosporin A, Sandopan B®, and Almond oil.

Another embodiment comprises cyclosporin A, Sandopan B Modified®, and Almond oil.

Another embodiment comprises cyclosporin A, Sandopan LS-24 Gel®, and Almond oil.

Another embodiment comprises cyclosporin A, Surtine T-A®, and Almond oil.

Another embodiment comprises cyclosporin A, a Carboxylic Acid/Fatty Acid, and Almond oil.

Another embodiment comprises cyclosporin A, Colaterge RAM®, and Almond oil.

Another embodiment comprises cyclosporin A, Colatrope INC®, and Almond oil.

Another embodiment comprises cyclosporin A, Crodacid B®, and Almond oil.

Another embodiment comprises cyclosporin A, DeTrope CA-100®, and Almond oil.

Another embodiment comprises cyclosporin A, Latol MTO®, and Almond oil.

Another embodiment comprises cyclosporin A, Lumulse CC-33 K®, and Almond oil.

Another embodiment comprises cyclosporin A, Mulls 2218®, and Almond oil.

Another embodiment comprises cyclosporin A, OL-600®, and Almond oil.

Another embodiment comprises cyclosporin A, OL-800®, and Almond oil.

Another embodiment comprises cyclosporin A, R-910®, and Almond oil.

Another embodiment comprises cyclosporin A, S-210®, and Almond oil.

Another embodiment comprises cyclosporin A, Sandopan DTC Acid®, and Almond oil.

Another embodiment comprises cyclosporin A, Sandopan LS 24 N®, and Almond oil.

Another embodiment comprises cyclosporin A, Sandopan MA-18®, and Almond oil.

Another embodiment comprises cyclosporin A, an Ethoxylated Alcohol, and Almond oil.

Another embodiment comprises cyclosporin A, Adsee 799®, and Almond oil.

Another embodiment comprises cyclosporin A, Adsee 799®, and Almond oil.

Another embodiment comprises cyclosporin A, Alfonic 610-3.5®, and Almond oil.

Another embodiment comprises cyclosporin A, Alfonic 810-2®, and Almond oil.

Another embodiment comprises cyclosporin A, Alfonic 810-6®, and Almond oil.

Another embodiment comprises cyclosporin A, Alfonic 1012-3®, and Almond oil.

Another embodiment comprises cyclosporin A, Alfonic 1012-5®, and Almond oil.

Another embodiment comprises cyclosporin A, Alfonic 1216CO-1.5®, and Almond oil.

Another embodiment comprises cyclosporin A, Alfonic 1216CO-7®, and Almond oil.

Another embodiment comprises cyclosporin A, Alfonic 1412-3®, and Almond oil.

Another embodiment comprises cyclosporin A, Alfonic 1412-7®, and Almond oil.

Another embodiment comprises cyclosporin A, Arlasolve 200®, and Almond oil.

Another embodiment comprises cyclosporin A, Arlasolve 200 Liquid®, and Almond oil.

Another embodiment comprises cyclosporin A, Armix 180-C®, and Almond oil.

Another embodiment comprises cyclosporin A, Armix 183®, and Almond oil.

Another embodiment comprises cyclosporin A, Armul 2404®, and Almond oil.

Another embodiment comprises cyclosporin A, Armul 2404®, and Almond oil.

Another embodiment comprises cyclosporin A, Atlas EMJ-C®, and Almond oil.

Another embodiment comprises cyclosporin A, Atlas G-2109®, and Almond oil.

Another embodiment comprises cyclosporin A, Atlas G-3886®, and Almond oil.

Another embodiment comprises cyclosporin A, Atlas G-3890®, and Almond oil.

Another embodiment comprises cyclosporin A, Bio Soft E-200®, and Almond oil.

Another embodiment comprises cyclosporin A, Bio Soft E-300®, and Almond oil.

Another embodiment comprises cyclosporin A, Bio Soft E-400®, and Almond oil.

Another embodiment comprises cyclosporin A, Bio Soft EN 600®, and Almond oil.

Another embodiment comprises cyclosporin A, Bio Soft TD-400®, and Almond oil.

Another embodiment comprises cyclosporin A, Bio Soft TD-630®, and Almond oil.

Another embodiment comprises cyclosporin A, Brij 30®, and Almond oil.

Another embodiment comprises cyclosporin A, Brij 52®, and Almond oil.

Another embodiment comprises cyclosporin A, Brij 56®, and Almond oil.

Another embodiment comprises cyclosporin A, Brij 58®, and Almond oil.

Another embodiment comprises cyclosporin A, Brij 72®, and Almond oil.

Another embodiment comprises cyclosporin A, Brij 76®, and Almond oil.

Another embodiment comprises cyclosporin A, Brij 78®, and Almond oil.

Another embodiment comprises cyclosporin A, Brij 93®, and Almond oil.

Another embodiment comprises cyclosporin A, Brij 97®, and Almond oil.

Another embodiment comprises cyclosporin A, Brij 98®, and Almond oil.

Another embodiment comprises cyclosporin A, Brij 700®, and Almond oil.

Another embodiment comprises cyclosporin A, Brij 700 S®, and Almond oil.

Another embodiment comprises cyclosporin A, Brij 721®, and Almond oil.

Another embodiment comprises cyclosporin A, Brij 721 S®, and Almond oil.

Another embodiment comprises cyclosporin A, Burcoterge CDG®, and Almond oil.

Another embodiment comprises cyclosporin A, Canasol AT 600®, and Almond oil.

Another embodiment comprises cyclosporin A, Canasol AT 800®, and Almond oil.

Another embodiment comprises cyclosporin A, Canasol AT 1200®, and Almond oil.

Another embodiment comprises cyclosporin A, Canasol BJ 35®, and Almond oil.

Another embodiment comprises cyclosporin A, Canasol BJ 36®, and Almond oil.

Another embodiment comprises cyclosporin A, Canasol BJ 52®, and Almond oil.

Another embodiment comprises cyclosporin A, Canasol BJ 58®, and Almond oil.

Another embodiment comprises cyclosporin A, Canasol BJ 72®, and Almond oil.

Another embodiment comprises cyclosporin A, Canasol BJ 78®, and Almond oil.

Another embodiment comprises cyclosporin A, Canasol BJ 98®, and Almond oil.

Another embodiment comprises cyclosporin A, Canasol BJ 307®, and Almond oil.

Another embodiment comprises cyclosporin A, Cerfak 1400®, and Almond oil.

Another embodiment comprises cyclosporin A, Cetomacrogol 1000 BP®, and Almond oil.

Another embodiment comprises cyclosporin A, Chemonic C-2®, and Almond oil.

Another embodiment comprises cyclosporin A, Chemonic C-10®, and Almond oil.

Another embodiment comprises cyclosporin A, Chemonic C-20®, and Almond oil.

Another embodiment comprises cyclosporin A, Chemonic CT-12®, and Almond oil.

Another embodiment comprises cyclosporin A, Chemonic CT-20®, and Almond oil.

Another embodiment comprises cyclosporin A, Chemonic CT-30®, and Almond oil.

Another embodiment comprises cyclosporin A, Chemonic CT-55®, and Almond oil.

Another embodiment comprises cyclosporin A, Chemonic G-7®, and Almond oil.

Another embodiment comprises cyclosporin A, Chemonic G-26®, and Almond oil.

Another embodiment comprises cyclosporin A, Chemonic L-4®, and Almond oil.

Another embodiment comprises cyclosporin A, Chemonic L-7®, and Almond oil.

Another embodiment comprises cyclosporin A, Chemonic L-12®, and Almond oil.

Another embodiment comprises cyclosporin A, Chemonic L-23®, and Almond oil.

Another embodiment comprises cyclosporin A, Chemonic O-2®, and Almond oil.

Another embodiment comprises cyclosporin A, Chemonic O-5®, and Almond oil.

Another embodiment comprises cyclosporin A, Chemonic O-10®, and Almond oil.

Another embodiment comprises cyclosporin A, Chemonic O-20®, and Almond oil.

Another embodiment comprises cyclosporin A, Chembnic S-2®, and Almond oil.

Another embodiment comprises cyclosporin A, Chemonic S-10®, and Almond oil.

Another embodiment comprises cyclosporin A, Chemonic S-20®, and Almond oil.

Another embodiment comprises cyclosporin A, Colamulse FE®, and Almond oil.

Another embodiment comprises cyclosporin A, Cremophor A 20®, and Almond oil.

Another embodiment comprises cyclosporin A, Cremophor SA 2®, and Almond oil.

Another embodiment comprises cyclosporin A, Dehydol 100®, and Almond oil.

Another embodiment comprises cyclosporin A, Dehydol O-4®, and Almond oil.

Another embodiment comprises cyclosporin A, Delonic C-18®, and Almond oil.

Another embodiment comprises cyclosporin A, DeSonic 6T®, and Almond oil.

Another embodiment comprises cyclosporin A, DeSonic 9D®, and Almond oil.

Another embodiment comprises cyclosporin A, DeSonic 9T®, and Almond oil.

Another embodiment comprises cyclosporin A, DeSonic 12D®, and Almond oil.

Another embodiment comprises cyclosporin A, DeSonic 12T®, and Almond oil.

Another embodiment comprises cyclosporin A, DeSonic 15T®, and Almond oil.

Another embodiment comprises cyclosporin A, DeSonic TDA-9®, and Almond oil.

Another embodiment comprises cyclosporin A, DeThox GLG-7®, and Almond oil.

Another embodiment comprises cyclosporin A, DeThox GLG-26®, and Almond oil.

Another embodiment comprises cyclosporin A, DeThox LA-4®, and Almond oil.

Another embodiment comprises cyclosporin A, DeThox LA-23®, and Almond oil.

Another embodiment comprises cyclosporin A, DeThox SA-80®, and Almond oil.

Another embodiment comprises cyclosporin A, Disponil O5®, and Almond oil.

Another embodiment comprises cyclosporin A, Eccoterge EO-41 B®, and Almond oil.

Another embodiment comprises cyclosporin A, Empilan KA2.5/90®, and Almond oil.

Another embodiment comprises cyclosporin A, Empilan KA5/90®, and Almond oil.

Another embodiment comprises cyclosporin A, Empilan KM-20®, and Almond oil.

Another embodiment comprises cyclosporin A, Empilan KM-50®, and Almond oil.

Another embodiment comprises cyclosporin A, Empilan L-23®, and Almond oil.

Another embodiment comprises cyclosporin A, Ethylan 25-3®, and Almond oil.

Another embodiment comprises cyclosporin A, Ethylan 1204®, and Almond oil.

Another embodiment comprises cyclosporin A, Ethylan DA-4®, and Almond oil.

Another embodiment comprises cyclosporin A, Ethylan LA-230®, and Almond oil.

Another embodiment comprises cyclosporin A, Ethylan SN®, and Almond oil.

Another embodiment comprises cyclosporin A, Ethylan TD-60®, and Almond oil.

Another embodiment comprises cyclosporin A, Ethylan TD-100®, and Almond oil.

Another embodiment comprises cyclosporin A, Ethylan TD-1407®, and Almond oil.

Another embodiment comprises cyclosporin A, Eumulgin B1®, and Almond oil.

Another embodiment comprises cyclosporin A, Eumulgin B2®, and Almond oil.

Another embodiment comprises cyclosporin A, Eumulgin B3®, and Almond oil.

Another embodiment comprises cyclosporin A, Eumulgin O-10®, and Almond oil.

Another embodiment comprises cyclosporin A, Flo Mo 80/20®, and Almond oil.

Another embodiment comprises cyclosporin A, Flo Mo Low Foam®, and Almond oil.

Another embodiment comprises cyclosporin A, Forlan C-24®, and Almond oil.

Another embodiment comprises cyclosporin A, Genapol 1454®, and Almond oil.

Another embodiment comprises cyclosporin A, Genapol BA-020®, and Almond oil.

Another embodiment comprises cyclosporin A, Genapol BA-040®, and Almond oil.

Another embodiment comprises cyclosporin A, Genapol C-100®, and Almond oil.

Another embodiment comprises cyclosporin A, Genapol DA 060®, and Almond oil.

Another embodiment comprises cyclosporin A, Genapol HS 020®, and Almond oil.

Another embodiment comprises cyclosporin A, Genapol HS 200®, and Almond oil.

Another embodiment comprises cyclosporin A, Genapol ID-040®, and Almond oil.

Another embodiment comprises cyclosporin A, Genapol ID-060®, and Almond oil.

Another embodiment comprises cyclosporin A, Genapol ID-090®, and Almond oil.

Another embodiment comprises cyclosporin A, Genapol LA 010®, and Almond oil.

Another embodiment comprises cyclosporin A, Genapol LA 020®, and Almond oil.

Another embodiment comprises cyclosporin A, Genapol LA 030®, and Almond oil.

Another embodiment comprises cyclosporin A, Genapol LA 040®, and Almond oil.

Another embodiment comprises cyclosporin A, Genapol LA 050®, and Almond oil.

Another embodiment comprises cyclosporin A, Genapol LA 060®, and Almond oil.

Another embodiment comprises cyclosporin A, Genapol LA 070®, and Almond oil.

Another embodiment comprises cyclosporin A, Genapol LA 070S®, and Almond oil.

Another embodiment comprises cyclosporin A, Genapol LA 230®, and Almond oil.

Another embodiment comprises cyclosporin A, Genapol O 020®, and Almond oil.

Another embodiment comprises cyclosporin A, Genapol O 050®, and Almond oil.

Another embodiment comprises cyclosporin A, Genapol O 100®, and Almond oil.

Another embodiment comprises cyclosporin A, Genapol O 200®, and Almond oil.

Another embodiment comprises cyclosporin A, Genapol SA 030®, and Almond oil.

Another embodiment comprises cyclosporin A, Genapol SA 120®, and Almond oil.

Another embodiment comprises cyclosporin A, Genapol T-020®, and Almond oil.

Another embodiment comprises cyclosporin A, Genapol UD-030®, and Almond oil.

Another embodiment comprises cyclosporin A, Genapol UD-050®, and Almond oil.

Another embodiment comprises cyclosporin A, Genapol UD-070®, and Almond oil.

Another embodiment comprises cyclosporin A, Genapol UD-079®, and Almond oil.

Another embodiment comprises cyclosporin A, Genapol UD-080®, and Almond oil.

Another embodiment comprises cyclosporin A, Genapol UD-110®, and Almond oil.

Another embodiment comprises cyclosporin A, Genapol X 030®, and Almond oil.

Another embodiment comprises cyclosporin A, Genapol X 050®, and Almond oil.

Another embodiment comprises cyclosporin A, Genapol X 060®, and Almond oil.

Another embodiment comprises cyclosporin A, Genapol X 070®, and Almond oil.

Another embodiment comprises cyclosporin A, Genapol X 080®, and Almond oil.

Another embodiment comprises cyclosporin A, Genapol X 100®, and Almond oil.

Another embodiment comprises cyclosporin A, Genapol X159®, and Almond oil.

Another embodiment comprises cyclosporin A, Generol 122 E5®, and Almond oil.

Another embodiment comprises cyclosporin A, Generol 122 E25®, and Almond oil.

Another embodiment comprises cyclosporin A, Hostacerin T-3®, and Almond oil.

Another embodiment comprises cyclosporin A, Iconol LF 110®, and Almond oil.

Another embodiment comprises cyclosporin A, Incropol CS-20®, and Almond oil.

Another embodiment comprises cyclosporin A, Lexemul CS-20®, and Almond oil.

Another embodiment comprises cyclosporin A, Liponic EG-1®, and Almond oil.

Another embodiment comprises cyclosporin A, Lipowax D®, and Almond oil.

Another embodiment comprises cyclosporin A, Lipowax G®, and Almond oil.

Another embodiment comprises cyclosporin A, Lipowax NI®, and Almond oil.

Another embodiment comprises cyclosporin A, Lipowax P®, and Almond oil.

Another embodiment comprises cyclosporin A, Lipowax P-31®, and Almond oil.

Another embodiment comprises cyclosporin A, Lipowax PR®, and Almond oil.

Another embodiment comprises cyclosporin A, Lumulse CS-20®, and Almond oil.

Another embodiment comprises cyclosporin A, Macol CSA-20®, and Almond oil.

Another embodiment comprises cyclosporin A, Marlox B 24/50®, and Almond oil.

Another embodiment comprises cyclosporin A, Mazawet 77®, and Almond oil.

Another embodiment comprises cyclosporin A, Norfox 1713®, and Almond oil.

Another embodiment comprises cyclosporin A, Norfox 2579®, and Almond oil.

Another embodiment comprises cyclosporin A, Norfox Lo Foam®, and Almond oil.

Another embodiment comprises cyclosporin A, Promulgen D®, and Almond oil.

Another embodiment comprises cyclosporin A, Promulgen G®, and Almond oil.

Another embodiment comprises cyclosporin A, Renex 30®, and Almond oil.

Another embodiment comprises cyclosporin A, Renex 36®, and Almond oil.

Another embodiment comprises cyclosporin A, Rhodasurf A 24®, and Almond oil.

Another embodiment comprises cyclosporin A, Rhodasurf AAE-10®, and Almond oil.

Another embodiment comprises cyclosporin A, Rhodasurf BEH-25®, and Almond oil.

Another embodiment comprises cyclosporin A, Rhodasurf BEH-40®, and Almond oil.

Another embodiment comprises cyclosporin A, Rhodasurf DA 530®, and Almond oil.

Another embodiment comprises cyclosporin A, Rhodasurf DA 630®, and Almond oil.

Another embodiment comprises cyclosporin A, Rhodasurf DA 639®, and Almond oil.

Another embodiment comprises cyclosporin A, Rhodasurf LAN-23®, and Almond oil.

Another embodiment comprises cyclosporin Ai Rhodasurf ON-870®, and Almond oil.

Another embodiment comprises cyclosporin A, Rhodasurf ON-877®, and Almond oil.

Another embodiment comprises cyclosporin A, Rhodasurf TB-970 FLK®, and Almond oil.

Another embodiment comprises cyclosporin A, Ritacet-20®, and Almond oil.

Another embodiment comprises cyclosporin A, Ritachol 1000®, and Almond oil.

Another embodiment comprises cyclosporin A, Ritachol 2000®, and Almond oil.

Another embodiment comprises cyclosporin A, Ritachol 5000®, and Almond oil.

Another embodiment comprises cyclosporin A, Ritox 35®, and Almond oil.

Another embodiment comprises cyclosporin A, Surfonic DA-4®, and Almond oil.

Another embodiment comprises cyclosporin A, Sunonic DA-6®, and Almond oil.

Another embodiment comprises cyclosporin A, Surfonic L46-7®, and Almond oil.

Another embodiment comprises cyclosporin A, Surfonic POA®, and Almond oil.

Another embodiment comprises cyclosporin A, Synthrapol KB®, and Almond oil.

Another embodiment comprises cyclosporin A, Teginacid®, and Almond oil.

Another embodiment comprises cyclosporin A, Teginacid C®, and Almond oil.

Another embodiment comprises cyclosporin A, Tegotens EC 11®, and Almond oil.

Another embodiment comprises cyclosporin A, Tinegal NA®, and Almond oil.

Another embodiment comprises cyclosporin A, Tomadol 400®, and Almond oil.

Another embodiment comprises cyclosporin A, Tomadol 600®, and Almond oil.

Another embodiment comprises cyclosporin A, Tomadol 900®, and Almond oil.

Another embodiment comprises cyclosporin A, Uniperol O®, and Almond oil.

Another embodiment comprises cyclosporin A, Witconol SN Series®, and Almond oil.

Another embodiment comprises cyclosporin A, an Ethoxylated Alkylphenol, and Almond oil.

Another embodiment comprises cyclosporin A, Antarox LF-222®, and Almond oil.

Another embodiment comprises cyclosporin A, Atlox 775®, and Almond oil.

Another embodiment comprises cyclosporin A, Caloxylate N-9®, and Almond oil.

Another embodiment comprises cyclosporin A, Canasol NF-1000®, and Almond oil.

Another embodiment comprises cyclosporin A, Canasol NF-3000®, and Almond oil.

Another embodiment comprises cyclosporin A, Canasol NF-3070®, and Almond oil.

Another embodiment comprises cyclosporin A, Canasol OF 1670®, and Almond oil.

Another embodiment comprises cyclosporin A, Canasol OF 2570®, and Almond oil.

Another embodiment comprises cyclosporin A, Canasol OF 4070®, and Almond oil.

Another embodiment comprises cyclosporin A, Chemax DNP-8®, and Almond oil.

Another embodiment comprises cyclosporin A, Chemax DNP-18®, and Almond oil.

Another embodiment comprises cyclosporin A, Chemax DNP-150/50®, and Almond oil.

Another embodiment comprises cyclosporin A, DeSonic 1.5N®, and Almond oil.

Another embodiment comprises cyclosporin A, DeSonic 4N®, and Almond oil.

Another embodiment comprises cyclosporin A, DeSonic 5N®, and Almond oil.

Another embodiment comprises cyclosporin A, DeSonic 6D®, and Almond oil.

Another embodiment comprises cyclosporin A, DeSonic 6N®, and Almond oil.

Another embodiment comprises cyclosporin A, DeSonic 7N®, and Almond oil.

Another embodiment comprises cyclosporin A, DeSonic 9N®, and Almond oil.

Another embodiment comprises cyclosporin A, DeSonic 10D®, and Almond oil.

Another embodiment comprises cyclosporin A, DeSonic 11N®, and Almond oil.

Another embodiment comprises cyclosporin A, DeSonic 12N®, and Almond oil.

Another embodiment comprises cyclosporin A, DeSonic 15N®, and Almond oil.

Another embodiment comprises cyclosporin A, DeSonic 20N®, and Almond oil.

Another embodiment comprises cyclosporin A, Eccoscour RC®, and Almond oil.

Another embodiment comprises cyclosporin A, Eccoterge EO-100®, and Almond oil.

Another embodiment comprises cyclosporin A, Emulsifier 632/90%®, and Almond oil.

Another embodiment comprises cyclosporin A, Geronol AG-821®, and Almond oil.

Another embodiment comprises cyclosporin A, Gradonic N-95®, and Almond oil.

Another embodiment comprises cyclosporin A, Hetoxide NP-4®, and Almond oil.

Another embodiment comprises cyclosporin A, Hetoxide NP-30®, and Almond oil.

Another embodiment comprises cyclosporin A, Hostapal N-100®, and Almond oil.

Another embodiment comprises cyclosporin A, Hostapal N-110®, and Almond oil.

Another embodiment comprises cyclosporin A, Igepal CTA-639W®, and Almond oil.

Another embodiment comprises cyclosporin A, Igepal DAP-9®, and Almond oil.

Another embodiment comprises cyclosporin A, Igepal OD-410®, and Almond oil.

Another embodiment comprises cyclosporin A, Igepal SS-837®, and Almond oil.

Another embodiment comprises cyclosporin A, Lipocol NP-9 USP®, and Almond oil.

Another embodiment comprises cyclosporin A, Macol DNP-10®, and Almond oil.

Another embodiment comprises cyclosporin A, Marlophen NP 5®, and Almond oil.

Another embodiment comprises cyclosporin A, Marlophen P 1®, and Almond oil.

Another embodiment comprises cyclosporin A, Surfonic NB®, and Almond oil.

Another embodiment comprises cyclosporin A, Surfonic OPB-307®, and Almond oil.

Another embodiment comprises cyclosporin A, Surfonic OPB-407®, and Almond oil.

Another embodiment comprises cyclosporin A, Syn Fac 334®, and Almond oil.

Another embodiment comprises cyclosporin A, Syn Fac 8216®, and Almond oil.

Another embodiment comprises cyclosporin A, Triton N-57®, and Almond oil.

Another embodiment comprises cyclosporin A, Trycol 6956®, and Almond oil.

Another embodiment comprises cyclosporin A, Trycol 6961®, and Almond oil.

Another embodiment comprises cyclosporin A, Trycol 6964®, and Almond oil.

Another embodiment comprises cyclosporin A, Trycol 6969®, and Almond oil.

Another embodiment comprises cyclosporin A, Trycol 6974®, and Almond oil.

Another embodiment comprises cyclosporin A, Witbreak DRB-127®, and Almond oil.

Another embodiment comprises cyclosporin A, Witbreak DRB-127®, and Almond oil.

Another embodiment comprises cyclosporin A, Witconol NP Series®, and Almond oil.

Another embodiment comprises cyclosporin A, an Ethoxylated Aryl Phenol, and Almond oil.

Another embodiment comprises cyclosporin A, Soprophor BSU®, and Almond oil.

Another embodiment comprises cyclosporin A, Soprophor CY/8®, and Almond oil.

Another embodiment comprises cyclosporin A, Soprophor S/25®, and Almond oil.

Another embodiment comprises cyclosporin A, Witconol NIO®, and Almond oil.

Another embodiment comprises cyclosporin A, Witconol NIW®, and Almond oil.

Another embodiment comprises cyclosporin A, Witconol S-100®, and Almond oil.

Another embodiment comprises cyclosporin A, an Ethoxylated Fatty Acid, and Almond oil.

Another embodiment comprises cyclosporin A, Aldo PGHMS®, and Almond oil.

Another embodiment comprises cyclosporin A, Alkamuls TO-15/HR®, and Almond oil.

Another embodiment comprises cyclosporin A, Armotan AL-69-66®, and Almond oil.

Another embodiment comprises cyclosporin A, Cerasynt 840®, and Almond oil.

Another embodiment comprises cyclosporin A, Cerasynt 945®, and Almond oil.

Another embodiment comprises cyclosporin A, Crystal Inhibitor No. 5®, and Almond oil.

Another embodiment comprises cyclosporin A, DeThox Acid L-9®, and Almond oil.

Another embodiment comprises cyclosporin A, DeThox Acid S-8®, and Almond oil.

Another embodiment comprises cyclosporin A, Ethofat 242/25®, and Almond oil.

Another embodiment comprises cyclosporin A, Hydropalat 65®, and Almond oil.

Another embodiment comprises cyclosporin A, Lipo EGMS®, and Almond oil.

Another embodiment comprises cyclosporin A, Lipopeg 2 DL®, and Almond oil.

Another embodiment comprises cyclosporin A, Lipopeg 4 DL®, and Almond oil.

Another embodiment comprises cyclosporin A, Lipopeg 4-L®, and Almond oil.

Another embodiment comprises cyclosporin A, Lipopeg 39-S®, and Almond oil.

Another embodiment comprises cyclosporin A, Lipopeg 4-S®, and Almond oil.

Another embodiment comprises cyclosporin A, Lipopeg 10-S®, and Almond oil.

Another embodiment comprises cyclosporin A, Lipopeg 100-S®, and Almond oil.

Another embodiment comprises cyclosporin A, Lipopeg 6000 DS®, and Almond oil.

Another embodiment comprises cyclosporin A, Lumulse 40-L®, and Almond oil.

Another embodiment comprises cyclosporin A, Lumulse 40-S®, and Almond oil.

Another embodiment comprises cyclosporin A, Lumulse 42-L®, and Almond oil.

Another embodiment comprises cyclosporin A, Lumulse 42-S®, and Almond oil.

Another embodiment comprises cyclosporin A, Lumulse 100-S®, and Almond oil.

Another embodiment comprises cyclosporin A, Lumulse 602-S®, and Almond oil.

Another embodiment comprises cyclosporin A, Magrabar PGE-20-0®, and Almond oil.

Another embodiment comprises cyclosporin A, Magrabar PGE-20L®, and Almond oil.

Another embodiment comprises cyclosporin A, Magrabar PGE-20T®, and Almond oil.

Another embodiment comprises cyclosporin A, Magrabar PGE-22-0®, and Almond oil.

Another embodiment comprises cyclosporin A, Magrabar PGE-22L®, and Almond oil.

Another embodiment comprises cyclosporin A, Magrabar PGE-22T®, and Almond oil.

Another embodiment comprises cyclosporin A, Magrabar PGE-40-0®, and Almond oil.

Another embodiment comprises cyclosporin A, Magrabar PGE-40L®, and Almond oil.

Another embodiment comprises cyclosporin A, Magrabar PGE-40T®, and Almond oil.

Another embodiment comprises cyclosporin A, Magrabar PGE-42-0®, and Almond oil.

Another embodiment comprises cyclosporin A, Magrabar PGE-42L®, and Almond oil.

Another embodiment comprises cyclosporin A, Magrabar PGE-42T®, and Almond oil.

Another embodiment comprises cyclosporin A, Magrabar PGE-60-0®, and Almond oil.

Another embodiment comprises cyclosporin A, Magrabar PGE-60L®, and Almond oil.

Another embodiment comprises cyclosporin A, Magrabar PGE-60T®, and Almond oil.

Another embodiment comprises cyclosporin A, Magrabar PGE-62-0®, and Almond oil.

Another embodiment comprises cyclosporin A, Magrabar PGE-62L®, and Almond oil.

Another embodiment comprises cyclosporin A, Magrabar PGE-62T®, and Almond oil.

Another embodiment comprises cyclosporin A, Mapeg S-40K®, and Almond oil.

Another embodiment comprises cyclosporin A, Marlowet OTS®, and Almond oil.

Another embodiment comprises cyclosporin A, Naturechem PGR®, and Almond oil.

Another embodiment comprises cyclosporin A, PG No. 4®, and Almond oil.

Another embodiment comprises cyclosporin A, Renex 20®, and Almond oil.

Another embodiment comprises cyclosporin A, Ritox 52®, and Almond oil.

Another embodiment comprises cyclosporin A, Ritox 53®, and Almond oil.

Another embodiment comprises cyclosporin A, Ritox 59®, and Almond oil.

Another embodiment comprises cyclosporin A, Surfax 8916/A®, and Almond oil.

Another embodiment comprises cyclosporin A, Tego Acid S 40 P®, and Almond oil.

Another embodiment comprises cyclosporin A, Tego Acid S 100 P®, and Almond oil.

Another embodiment comprises cyclosporin A, Tween 20®, and Almond oil.

Another embodiment comprises cyclosporin A, Volpo 131®, and Almond oil.

Another embodiment comprises cyclosporin A, an Ethoxylated Fatty Ester or Oil, and Almond oil.

Another embodiment comprises cyclosporin A, Acconon 6-C10®, and Almond oil.

Another embodiment comprises cyclosporin A, Acconon CC-6®, and Almond oil.

Another embodiment comprises cyclosporin A, Acconon CO-7®, and Almond oil.

Another embodiment comprises cyclosporin A, Aldosperse 40/60 FG®, and Almond oil.

Another embodiment comprises cyclosporin A, Aldosperse ML-23®, and Almond oil.

Another embodiment comprises cyclosporin A, Aldosperse MS-20 FG®, and Almond oil.

Another embodiment comprises cyclosporin A, Alkamuls EL-620®, and Almond oil.

Another embodiment comprises cyclosporin A, Alkamuls EL-719®, and Almond oil.

Another embodiment comprises cyclosporin A, Alkamuls EL-985®, and Almond oil.

Another embodiment comprises cyclosporin A, Arlatone G®, and Almond oil.

Another embodiment comprises cyclosporin A, Arlatone T®, and Almond oil.

Another embodiment comprises cyclosporin A, Atlas G-1045A®, and Almond oil.

Another embodiment comprises cyclosporin A, Atlas G-1086®, and Almond oil.

Another embodiment comprises cyclosporin A, Atlas G-1087®, and Almond oil.

Another embodiment comprises cyclosporin A, Atlas G-1089®, and Almond oil.

Another embodiment comprises cyclosporin A, Atlas G-1096®, and Almond oil.

Another embodiment comprises cyclosporin A, Atlas G-1292®, and Almond oil.

Another embodiment comprises cyclosporin A, Atlas G-1293®, and Almond oil.

Another embodiment comprises cyclosporin A, Atlas G-1300®, and Almond oil.

Another embodiment comprises cyclosporin A, Atlas G-7076®, and Almond oil.

Another embodiment comprises cyclosporin A, Capmul EMG®, and Almond oil.

Another embodiment comprises cyclosporin A, Chemonic CO-40®, and Almond oil.

Another embodiment comprises cyclosporin A, Chemonic LI-3®, and Almond oil.

Another embodiment comprises cyclosporin A, Chemonic LI-7®, and Almond oil.

Another embodiment comprises cyclosporin A, Cirrasol GM®, and Almond oil.

Another embodiment comprises cyclosporin A, Cremophor CO 40®, and Almond oil.

Another embodiment comprises cyclosporin A, Cremophor CO 410®, and Almond oil.

Another embodiment comprises cyclosporin A, Cremophor EL®, and Almond oil.

Another embodiment comprises cyclosporin A, Cremophor GC7®, and Almond oil.

Another embodiment comprises cyclosporin A, Cremophor RH-40®, and Almond oil.

Another embodiment comprises cyclosporin A, Crovol A-40®, and Almond oil.

Another embodiment comprises cyclosporin A, Crovol A-70®, and Almond oil.

Another embodiment comprises cyclosporin A, Crovol M-70®, and Almond oil.

Another embodiment comprises cyclosporin A, Crovol PK-70®, and Almond oil.

Another embodiment comprises cyclosporin A, Cutina E-24®, and Almond oil.

Another embodiment comprises cyclosporin A, Dacospin 12-R®, and Almond oil.

Another embodiment comprises cyclosporin A, Dehymuls HRE-7®, and Almond oil.

Another embodiment comprises cyclosporin A, DeSonic 30C®, and Almond oil.

Another embodiment comprises cyclosporin A, DeSonic 36C®, and Almond oil.

Another embodiment comprises cyclosporin A, DeSonic 40C®, and Almond oil.

Another embodiment comprises cyclosporin A, Durfax 60®, and Almond oil.

Another embodiment comprises cyclosporin A, Durfax 65®, and Almond oil.

Another embodiment comprises cyclosporin A, Durfax 80®, and Almond oil.

Another embodiment comprises cyclosporin A, Durfax EOM®, and Almond oil.

Another embodiment comprises cyclosporin A, Eccoterge NF-2®, and Almond oil.

Another embodiment comprises cyclosporin A, Emulpon CO-360®, and Almond oil.

Another embodiment comprises cyclosporin A, Emulpon CO-550®, and Almond oil.

Another embodiment comprises cyclosporin A, Emulsogen EL®, and Almond oil.

Another embodiment comprises cyclosporin A, Emulsogen HCO 040®, and Almond oil.

Another embodiment comprises cyclosporin A, Emulsogen HCO 060®, and Almond oil.

Another embodiment comprises cyclosporin A, Emulsynt 1055®, and Almond oil.

Another embodiment comprises cyclosporin A, Ethox 3095®, and Almond oil.

Another embodiment comprises cyclosporin A, Eumulgin RO-40®, and Almond oil.

Another embodiment comprises cyclosporin A, Genapol G-260®, and Almond oil.

Another embodiment comprises cyclosporin A, Glycosperse L-20®, and Almond oil.

Another embodiment comprises cyclosporin A, Glycosperse O-5®, and Almond oil.

Another embodiment comprises cyclosporin A, Glycosperse O-20®, and Almond oil.

Another embodiment comprises cyclosporin A, Glycosperse O-20 FG®, and Almond oil.

Another embodiment comprises cyclosporin A, Glycosperse S-20®, and Almond oil.

Another embodiment comprises cyclosporin A, Glycosperse S-20 FG®, and Almond oil.

Another embodiment comprises cyclosporin A, Glycosperse TS-20®, and Almond oil.

Another embodiment comprises cyclosporin A, Glycosperse TS-20 FG®, and Almond oil.

Another embodiment comprises cyclosporin A, Hetan SL®, and Almond oil.

Another embodiment comprises cyclosporin A, Hetan SO®, and Almond oil.

Another embodiment comprises cyclosporin A, Hetan SS®, and Almond oil.

Another embodiment comprises cyclosporin A, Hetoxide C-2®, and Almond oil.

Another embodiment comprises cyclosporin A, Hetoxide C-9®, and Almond oil.

Another embodiment comprises cyclosporin A, Hetoxide C-15®, and Almond oil.

Another embodiment comprises cyclosporin A, Hetoxide C-25®, and Almond oil.

Another embodiment comprises cyclosporin A, Hetoxide C-40®, and Almond oil.

Another embodiment comprises cyclosporin A, Hetoxide C-200®, and Almond oil.

Another embodiment comprises cyclosporin A, Hetoxide C-200-50%®, and Almond oil.

Another embodiment comprises cyclosporin A, Hetoxide GC-30®, and Almond oil.

Another embodiment comprises cyclosporin A, Hetoxide HC-60®, and Almond oil.

Another embodiment comprises cyclosporin A, Ice No. 2®, and Almond oil.

Another embodiment comprises cyclosporin A, Incrocas 30/40®, and Almond oil.

Another embodiment comprises cyclosporin A, Lexol EC®, and Almond oil.

Another embodiment comprises cyclosporin A, Lexol EO®, and Almond oil.

Another embodiment comprises cyclosporin A, Lipocol HCO-40®, and Almond oil.

Another embodiment comprises cyclosporin A, Lipocol HCO-60®, and Almond oil.

Another embodiment comprises cyclosporin A, Lipocol O-3 Special®, and Almond oil.

Another embodiment comprises cyclosporin A, Lipopeg 2-L®, and Almond oil.

Another embodiment comprises cyclosporin A, Lipopeg 4-DO®, and Almond oil.

Another embodiment comprises cyclosporin A, Lipopeg 4-DS®, and Almond oil.

Another embodiment comprises cyclosporin A, Lipovol GTB®, and Almond oil.

Another embodiment comprises cyclosporin A, Lonzest SML-20®, and Almond oil.

Another embodiment comprises cyclosporin A, Lonzest SMO-20®, and Almond oil.

Another embodiment comprises cyclosporin A, Lonzest SMS-20®, and Almond oil.

Another embodiment comprises cyclosporin A, Lonzest STO-20®, and Almond oil.

Another embodiment comprises cyclosporin A, Lonzest STS-20®, and Almond oil.

Another embodiment comprises cyclosporin A, Lumulse GR-40®, and Almond oil.

Another embodiment comprises cyclosporin A, Lumulse GRH-40®, and Almond oil.

Another embodiment comprises cyclosporin A, Lumulse POE (7) GML®, and Almond oil.

Another embodiment comprises cyclosporin A, Lumulse POE (12) Gly®, and Almond oil.

Another embodiment comprises cyclosporin A, Lumulse POE (40) MS KP®, and Almond oil.

Another embodiment comprises cyclosporin A, Marlowet 4750®, and Almond oil.

Another embodiment comprises cyclosporin A, Marlowet LVS®, and Almond oil.

Another embodiment comprises cyclosporin A, Marlowet R 11®, and Almond oil.

Another embodiment comprises cyclosporin A, Marlowet R 40®, and Almond oil.

Another embodiment comprises cyclosporin A, Mazol 80 MGk®, and Almond oil.

Another embodiment comprises cyclosporin A, Nonionic Emulsifier T-9®, and Almond oil.

Another embodiment comprises cyclosporin A, Oronal LCG®, and Almond oil.

Another embodiment comprises cyclosporin A, Polyderm PPI-CO-200®, and Almond oil.

Another embodiment comprises cyclosporin A, Polyderm PPI-CO-40®, and Almond oil.

Another embodiment comprises cyclosporin A, Rewoderm LI 520-70®, and Almond oil.

Another embodiment comprises cyclosporin A, Ritapeg 150 DS®, and Almond oil.

Another embodiment comprises cyclosporin A, Softigen 767®, and Almond oil.

Another embodiment comprises cyclosporin A, Surfactol 318®, and Almond oil.

Another embodiment comprises cyclosporin A, Surfactol 365®, and Almond oil.

Another embodiment comprises cyclosporin A, Syn Lube 107®, and Almond oil.

Another embodiment comprises cyclosporin A, Syn Lube 728®, and Almond oil.

Another embodiment comprises cyclosporin A, Syn Lube 1632H®, and Almond oil.

Another embodiment comprises cyclosporin A, Syn Lube 6277-A®, and Almond oil.

Another embodiment comprises cyclosporin A, T-Det C-20®, and Almond oil.

Another embodiment comprises cyclosporin A, T-Det C-40®, and Almond oil.

Another embodiment comprises cyclosporin A, Tally 100 Plus®, and Almond oil.

Another embodiment comprises cyclosporin A, Uniperol EL®, and Almond oil.

Another embodiment comprises cyclosporin A, a Fatty Ester, and Almond oil.

Another embodiment comprises cyclosporin A, Actralube-Syn 147®, and Almond oil.

Another embodiment comprises cyclosporin A, Atlas G-1556®, and Almond oil.

Another embodiment comprises cyclosporin A, Atlas G-1564®, and Almond oil.

Another embodiment comprises cyclosporin A, Atlasol Base Oil S®, and Almond oil.

Another embodiment comprises cyclosporin A, Base ML®, and Almond oil.

Another embodiment comprises cyclosporin A, Base MT®, and Almond oil.

Another embodiment comprises cyclosporin A, Cerasynt 303®, and Almond oil.

Another embodiment comprises cyclosporin A, Dermol 1012®, and Almond oil.

Another embodiment comprises cyclosporin A, Kemester 4000®, and Almond oil.

Another embodiment comprises cyclosporin A, Lactipol S®, and Almond oil.

Another embodiment comprises cyclosporin A, Magrabar PGO®, and Almond oil.

Another embodiment comprises cyclosporin A, Mayco Base BFO®, and Almond oil.

Another embodiment comprises cyclosporin A, Methyl Linoleate®, and Almond oil.

Another embodiment comprises cyclosporin A, Pationic 122A®, and Almond oil.

Another embodiment comprises cyclosporin A, Pationic 138C®, and Almond oil.

Another embodiment comprises cyclosporin A, Pationic CSL®, and Almond oil.

Another embodiment comprises cyclosporin A, Pationic ISL®, and Almond oil.

Another embodiment comprises cyclosporin A, Pationic SBL®, and Almond oil.

Another embodiment comprises cyclosporin A, Pationic SSL®, and Almond oil.

Another embodiment comprises cyclosporin A, Ritasol®, and Almond oil.

Another embodiment comprises cyclosporin A, Tego Alkanol CS 20®, and Almond oil.

Another embodiment comprises cyclosporin A, Tego Alkanol L23 P®, and Almond oil.

Another embodiment comprises cyclosporin A, Tego Alkanol S2®, and Almond oil.

Another embodiment comprises cyclosporin A, Tego Alkanol S20 P®, and Almond oil.

Another embodiment comprises cyclosporin A, Triemulsifier 600 MS®, and Almond oil.

Another embodiment comprises cyclosporin A, a Glycerol Esters, and Almond oil.

Another embodiment comprises cyclosporin A, Agro #9 Wint SBO®, and Almond oil.

Another embodiment comprises cyclosporin A, Ahcovel Base 700®, and Almond oil.

Another embodiment comprises cyclosporin A, Aldo HMS FG®, and Almond oil.

Another embodiment comprises cyclosporin A, Aldo MLD®, and Almond oil.

Another embodiment comprises cyclosporin A, Aldo MLD FG®, and Almond oil.

Another embodiment comprises cyclosporin A, Aldo MO FG®, and Almond oil.

Another embodiment comprises cyclosporin A, Aldo MS®, and Almond oil.

Another embodiment comprises cyclosporin A, Aldo MS FG®, and Almond oil.

Another embodiment comprises cyclosporin A, Aldo MS LG FG®, and Almond oil.

Another embodiment comprises cyclosporin A, Aldo MSD®, and Almond oil.

Another embodiment comprises cyclosporin A, Aldo MSD FG®, and Almond oil.

Another embodiment comprises cyclosporin A, Aldosperse O-20 FG®, and Almond oil.

Another embodiment comprises cyclosporin A, Aldosperse TS-20 FG®, and Almond oil.

Another embodiment comprises cyclosporin A, Aldosperse TS-40 FG®, and Almond oil.

Another embodiment comprises cyclosporin A, Arlacel 165®, and Almond oil.

Another embodiment comprises cyclosporin A, Arlacel 186®, and Almond oil.

Another embodiment comprises cyclosporin A, Capmul GMO®, and Almond oil.

Another embodiment comprises cyclosporin A, Capmul GMS®, and Almond oil.

Another embodiment comprises cyclosporin A, Caprol 3GO®, and Almond oil.

Another embodiment comprises cyclosporin A, Caprol 3GVS®, and Almond oil.

Another embodiment comprises cyclosporin A, Caprol 6G2S®, and Almond oil.

Another embodiment comprises cyclosporin A, Caprol 10G40®, and Almond oil.

Another embodiment comprises cyclosporin A, Caprol 10G100®, and Almond oil.

Another embodiment comprises cyclosporin A, Caprol ET®, and Almond oil.

Another embodiment comprises cyclosporin A, Caprol PGE860®, and Almond oil.

Another embodiment comprises cyclosporin A, Cerasynt 945®, and Almond oil.

Another embodiment comprises cyclosporin A, Cerasynt GMS®, and Almond oil.

Another embodiment comprises cyclosporin A, Cerasynt Q®, and Almond oil.

Another embodiment comprises cyclosporin A, Cerasynt SD®, and Almond oil.

Another embodiment comprises cyclosporin A, Cerasynt WM®, and Almond oil.

Another embodiment comprises cyclosporin A, Chemsperse 14®, and Almond oil.

Another embodiment comprises cyclosporin A, Cremophor GO-32®, and Almond oil.

Another embodiment comprises cyclosporin A, Cremophor GS11®, and Almond oil.

Another embodiment comprises cyclosporin A, Cremophor GS-32®, and Almond oil.

Another embodiment comprises cyclosporin A, Cutina KD-16®, and Almond oil.

Another embodiment comprises cyclosporin A, Dehymuls PGPH®, and Almond oil.

Another embodiment comprises cyclosporin A, Dermol DGDIS®, and Almond oil.

Another embodiment comprises cyclosporin A, Dermol DGMIS®, and Almond oil.

Another embodiment comprises cyclosporin A, Dermol G-76®, and Almond oil.

Another embodiment comprises cyclosporin A, Dermol G-7DI¢, and Almond oil.

Another embodiment comprises cyclosporin A, Dermol NGDI®, and Almond oil.

Another embodiment comprises cyclosporin A, Dermolan GLH®, and Almond oil.

Another embodiment comprises cyclosporin A, Drewmulse GMO®, and Almond oil.

Another embodiment comprises cyclosporin A, Drewpol 3-5-M®, and Almond oil.

Another embodiment comprises cyclosporin A, Durlac 100 W®, and Almond oil.

Another embodiment comprises cyclosporin A, Dur-Lo®, and Almond oil.

Another embodiment comprises cyclosporin A, Dynasan 118®, and Almond oil.

Another embodiment comprises cyclosporin A, EC-25®, and Almond oil.

Another embodiment comprises cyclosporin A, EM 40®, and Almond oil.

Another embodiment comprises cyclosporin A, Emerest 2400®, and Almond oil.

Another embodiment comprises cyclosporin A, Emerest 2452®, and Almond oil.

Another embodiment comprises cyclosporin A, Empilan G-26®, and Almond oil.

Another embodiment comprises cyclosporin A, Genapol TSM®, and Almond oil.

Another embodiment comprises cyclosporin A, Hostacerin DGI®, and Almond oil.

Another embodiment comprises cyclosporin A, Hostacerin DGL®, and Almond oil.

Another embodiment comprises cyclosporin A, Hostacerin DGMS®, and Almond oil.

Another embodiment comprises cyclosporin A, Hostacerin DGSB®, and Almond oil.

Another embodiment comprises cyclosporin A, Ice No. 2®, and Almond oil.

Another embodiment comprises cyclosporin A, Imwitor 742®, and Almond oil.

Another embodiment comprises cyclosporin A, Imwitor 780 K®, and Almond oil.

Another embodiment comprises cyclosporin A, Imwitor 960 Flakes®, and Almond oil.

Another embodiment comprises cyclosporin A, Isolan GI 34®, and Almond oil.

Another embodiment comprises cyclosporin A, Isolan GO 33®, and Almond oil.

Another embodiment comprises cyclosporin A, Kemester 1000®, and Almond oil.

Another embodiment comprises cyclosporin A, Kemester 2000®, and Almond oil.

Another embodiment comprises cyclosporin A, Kemester 2000®, and Almond oil.

Another embodiment comprises cyclosporin A, Kemester 6000SE®, and Almond oil.

Another embodiment comprises cyclosporin A, Lamecreme DGE 18®, and Almond oil.

Another embodiment comprises cyclosporin A, Lexemul 515®, and Almond oil.

Another embodiment comprises cyclosporin A, Lexemul 561®, and Almond oil.

Another embodiment comprises cyclosporin A, Lexemul AR®, and Almond oil.

Another embodiment comprises cyclosporin A, Lexemul AS®, and Almond oil.

Another embodiment comprises cyclosporin A, Lexemul GDL®, and Almond oil.

Another embodiment comprises cyclosporin A, Lexemul T®, and Almond oil.

Another embodiment comprises cyclosporin A, Lipomulse 165®, and Almond oil.

Another embodiment comprises cyclosporin A, Lumulse GML K®, and Almond oil.

Another embodiment comprises cyclosporin A, Lumulse GMO K®, and Almond oil.

Another embodiment comprises cyclosporin A, Lumulse GMR K®, and Almond oil.

Another embodiment comprises cyclosporin A, Lumulse GMT K®, and Almond oil.

Another embodiment comprises cyclosporin A, Magrabar GMC®, and Almond oil.

Another embodiment comprises cyclosporin A, Magrabar GMO-CK®, and Almond oil.

Another embodiment comprises cyclosporin A, Magrabar MDG-5050®, and Almond oil.

Another embodiment comprises cyclosporin A, Magrabar PGO-315®, and Almond oil.

Another embodiment comprises cyclosporin A, Magrabar PGO-1010®, and Almond oil.

Another embodiment comprises cyclosporin A, Mazol 300K®, and Almond oil.

Another embodiment comprises cyclosporin A, Mazol GMO-K®, and Almond oil.

Another embodiment comprises cyclosporin A, Mazol GMS-K®, and Almond oil.

Another embodiment comprises cyclosporin A, Mazol PG031-K®, and Almond oil.

Another embodiment comprises cyclosporin A, Miglyol 812®, and Almond oil.

Another embodiment comprises cyclosporin A, Norfox 165C®, and Almond oil.

Another embodiment comprises cyclosporin A, Schercemol GMIS®, and Almond oil.

Another embodiment comprises cyclosporin A, Tegin®, and Almond oil.

Another embodiment comprises cyclosporin A, Tegin 4100 Pellets®, and Almond oil.

Another embodiment comprises cyclosporin A, Tegin M Pellets®, and Almond oil.

Another embodiment comprises cyclosporin A, Tegin OV®, and Almond oil.

Another embodiment comprises cyclosporin A, Teginacid H®, and Almond oil.

Another embodiment comprises cyclosporin A, Tego Cosmo P813®, and Almond oil.

Another embodiment comprises cyclosporin A, Wickenol 535®, and Almond oil.

Another embodiment comprises cyclosporin A, Witconol 14®, and Almond oil.

Another embodiment comprises cyclosporin A, Witconol 14®, and Almond oil.

Another embodiment comprises cyclosporin A, Witconol 14F®, and Almond oil.

Another embodiment comprises cyclosporin A, Witconol 18L®, and Almond oil.

Another embodiment comprises cyclosporin A, Witconol GOT®, and Almond oil.

Another embodiment comprises cyclosporin A, Witconol MST®, and Almond oil.

Another embodiment comprises cyclosporin A, Witconol RHT®, and Almond oil.

Another embodiment comprises cyclosporin A, a Glycol Ester, and Almond oil.

Another embodiment comprises cyclosporin A, Alkamuls 600 DO®, and Almond oil.

Another embodiment comprises cyclosporin A, Alkamuls SEG®, and Almond oil.

Another embodiment comprises cyclosporin A, Atlas EM-2®, and Almond oil.

Another embodiment comprises cyclosporin A, Cerasynt IP®, and Almond oil.

Another embodiment comprises cyclosporin A, Cerasynt M®, and Almond oil.

Another embodiment comprises cyclosporin A, Cerasynt MN®, and Almond oil.

Another embodiment comprises cyclosporin A, Cerasynt PA®, and Almond oil.

Another embodiment comprises cyclosporin A, Chemsperse EGDS®, and Almond oil.

Another embodiment comprises cyclosporin A, Chemsperse EGMS®, and Almond oil.

Another embodiment comprises cyclosporin A, Colonial Monolaurin®, and Almond oil.

Another embodiment comprises cyclosporin A, DeMuls SGE-95®, and Almond oil.

Another embodiment comprises cyclosporin A, Eccoterge 200®, and Almond oil.

Another embodiment comprises cyclosporin A, Emerest 2380®, and Almond oil.

Another embodiment comprises cyclosporin A, Ethox 2610®, and Almond oil.

Another embodiment comprises cyclosporin A, Ethox DO-9®, and Almond oil.

Another embodiment comprises cyclosporin A, Ethox DO-14®, and Almond oil.

Another embodiment comprises cyclosporin A, Ethox SO-9®, and Almond oil.

Another embodiment comprises cyclosporin A, Fizul MD-318®, and Almond oil.

Another embodiment comprises cyclosporin A, Genapol EGDS-VHP®, and Almond oil.

Another embodiment comprises cyclosporin A, Genapol TS Powder®, and Almond oil.

Another embodiment comprises cyclosporin A, Hostacerin WO®, and Almond oil.

Another embodiment comprises cyclosporin A, Inversol 140®, and Almond oil.

Another embodiment comprises cyclosporin A, Kemester 104®, and Almond oil.

Another embodiment comprises cyclosporin A, Kemester 205®, and Almond oil.

Another embodiment comprises cyclosporin A, Kemester 226®, and Almond oil.

Another embodiment comprises cyclosporin A, Kemester 5221 SE®, and Almond oil.

Another embodiment comprises cyclosporin A, Kemester EGDS®, and Almond oil.

Another embodiment comprises cyclosporin A, Lexemul EGDS®, and Almond oil.

Another embodiment comprises cyclosporin A, Lexemul EGMS®, and Almond oil.

Another embodiment comprises cyclosporin A, Lexemul P®, and Almond oil.

Another embodiment comprises cyclosporin A, Lipo DGLS®, and Almond oil.

Another embodiment comprises cyclosporin A, Lipo EGDS®, and Almond oil.

Another embodiment comprises cyclosporin A, Lipo PGMS®, and Almond oil.

Another embodiment comprises cyclosporin A, Liposorb S-4®, and Almond oil.

Another embodiment comprises cyclosporin A, Liposorb TO-20®, and Almond oil.

Another embodiment comprises cyclosporin A, Lumulse PGO®, and Almond oil.

Another embodiment comprises cyclosporin A, Mackester EGDS®, and Almond oil.

Another embodiment comprises cyclosporin A, Mackester EGMS®, and Almond oil.

Another embodiment comprises cyclosporin A, Mackester GSTP®, and Almond oil.

Another embodiment comprises cyclosporin A, Mackester Series®, and Almond oil.

Another embodiment comprises cyclosporin A, Magrabar PDG-50®, and Almond oil.

Another embodiment comprises cyclosporin A, Mapeg 6000 DS®, and Almond oil.

Another embodiment comprises cyclosporin A, Marlowet 4702®, and Almond oil.

Another embodiment comprises cyclosporin A, Monalube 305®, and Almond oil.

Another embodiment comprises cyclosporin A, Monalube 310®, and Almond oil.

Another embodiment comprises cyclosporin A, Monalube 315®, and Almond oil.

Another embodiment comprises cyclosporin A, Monalube 320®, and Almond oil.

Another embodiment comprises cyclosporin A, Monalube 325®, and Almond oil.

Another embodiment comprises cyclosporin A, Monalube 330®, and Almond oil.

Another embodiment comprises cyclosporin A, Naturechem PGHS®, and Almond oil.

Another embodiment comprises cyclosporin A, Polycastorol PLO-840®, and Almond oil.

Another embodiment comprises cyclosporin A, Polytex 10M®, and Almond oil.

Another embodiment comprises cyclosporin A, Ritasynt IP®, and Almond oil.

Another embodiment comprises cyclosporin A, Ross Chem PEG 600 DT®, and Almond oil.

Another embodiment comprises cyclosporin A, Schercemol PGMS®, and Almond oil.

Another embodiment comprises cyclosporin A, Sponto H-44C®, and Almond oil.

Another embodiment comprises cyclosporin A, Tegin G®, and Almond oil.

Another embodiment comprises cyclosporin A, Witbreak DGE-182®, and Almond oil.

Another embodiment comprises cyclosporin A, Witbreak DGE-182®, and Almond oil.

Another embodiment comprises cyclosporin A, Witbreak DRA-21®, and Almond oil.

Another embodiment comprises cyclosporin A, Witbreak DRA-21®, and Almond oil.

Another embodiment comprises cyclosporin A, Witbreak DRA-50®, and Almond oil.

Another embodiment comprises cyclosporin A, Witbreak DRA-50®, and Almond oil.

Another embodiment comprises cyclosporin A, Witconol F26-46®, and Almond oil.

Another embodiment comprises cyclosporin A, Witconol H-32®, and Almond oil.

Another embodiment comprises cyclosporin A, Witconol H-33®, and Almond oil.

Another embodiment comprises cyclosporin A, Witconol H-35A®, and Almond oil.

Another embodiment comprises cyclosporin A, Witconol RHP®, and Almond oil.

Another embodiment comprises cyclosporin A, a Lanolin-based Derivative, and Almond oil.

Another embodiment comprises cyclosporin A, Amerchol CAB®, and Almond oil.

Another embodiment comprises cyclosporin A, Amerchol L-101®, and Almond oil.

Another embodiment comprises cyclosporin A, Amerlate LFA-LO®, and Almond oil.

Another embodiment comprises cyclosporin A, Amerlate P®, and Almond oil.

Another embodiment comprises cyclosporin A, Barre Common Degras®, and Almond oil.

Another embodiment comprises cyclosporin A, Cholesterol NF®, and Almond oil.

Another embodiment comprises cyclosporin A, Crodalan AWS®, and Almond oil.

Another embodiment comprises cyclosporin A, Crodalan LA®, and Almond oil.

Another embodiment comprises cyclosporin A, Emery 1650®, and Almond oil.

Another embodiment comprises cyclosporin A, Emery 1650®, and Almond oil.

Another embodiment comprises cyclosporin A, Emery 1740®, and Almond oil.

Another embodiment comprises cyclosporin A, Emery 1740®, and Almond oil.

Another embodiment comprises cyclosporin A, Forlan 500®, and Almond oil.

Another embodiment comprises cyclosporin A, Forlan L®, and Almond oil.

Another embodiment comprises cyclosporin A, Laneto 50®, and Almond oil.

Another embodiment comprises cyclosporin A, Laneto 100®, and Almond oil.

Another embodiment comprises cyclosporin A, Laneto AWS®, and Almond oil.

Another embodiment comprises cyclosporin A, Lanfrax 1776®, and Almond oil.

Another embodiment comprises cyclosporin A, Lanfrax 1776®, and Almond oil.

Another embodiment comprises cyclosporin A, Lanogel 21®, and Almond oil.

Another embodiment comprises cyclosporin A, Lipolan®, and Almond oil.

Another embodiment comprises cyclosporin A, Lipolan 31®, and Almond oil.

Another embodiment comprises cyclosporin A, OHlan®, and Almond oil.

Another embodiment comprises cyclosporin A, Polychol 5®, and Almond oil.

Another embodiment comprises cyclosporin A, Polychol 15®, and Almond oil.

Another embodiment comprises cyclosporin A, Ritacetyl®, and Almond oil.

Another embodiment comprises cyclosporin A, Ritachol®, and Almond oil.

Another embodiment comprises cyclosporin A, Ritahydrox®, and Almond oil.

Another embodiment comprises cyclosporin A, Ritalafa®, and Almond oil.

Another embodiment comprises cyclosporin A, Ritalan®, and Almond oil.

Another embodiment comprises cyclosporin A, Ritalan AWS®, and Almond oil.

Another embodiment comprises cyclosporin A, Ritalan C®, and Almond oil.

Another embodiment comprises cyclosporin A, Ritawax®, and Almond oil.

Another embodiment comprises cyclosporin A, Ritawax AEO®, and Almond oil.

Another embodiment comprises cyclosporin A, Ritawax ALA®, and Almond oil.

Another embodiment comprises cyclosporin A, Solan/Solan 50/Super Solan®, and Almond oil.

Another embodiment comprises cyclosporin A, Super Hartolan/Hartolan®, and Almond oil.

Another embodiment comprises cyclosporin A, Supersat AWS-4®, and Almond oil.

Another embodiment comprises cyclosporin A, Supersat AWS-24®, and Almond oil.

Another embodiment comprises cyclosporin A, a Lecithin or Lecithin Derivative, and Almond oil.

Another embodiment comprises cyclosporin A, Alcolec®, and Almond oil.

Another embodiment comprises cyclosporin A, Lecithin®, and Almond oil.

Another embodiment comprises cyclosporin A, Lexin K®, and Almond oil.

Another embodiment comprises cyclosporin A, Natipide®, and Almond oil.

Another embodiment comprises cyclosporin A, a Lignin or Lignin Derivative, and Almond oil.

Another embodiment comprises cyclosporin A, Diwatex XP 9®, and Almond oil.

Another embodiment comprises cyclosporin A, Dynasperse LCD®, and Almond oil.

Another embodiment comprises cyclosporin A, Indulin SAL®, and Almond oil.

Another embodiment comprises cyclosporin A, Indulin W-1®, and Almond oil.

Another embodiment comprises cyclosporin A, Indulin W-5®, and Almond oil.

Another embodiment comprises cyclosporin A, Lignosol FTA®, and Almond oil.

Another embodiment comprises cyclosporin A, Lignosol SFX-65®, and Almond oil.

Another embodiment comprises cyclosporin A, Marasperse 52 CP®, and Almond oil.

Another embodiment comprises cyclosporin A, Marasperse AG®, and Almond oil.

Another embodiment comprises cyclosporin A, Marasperse CBOS-4®, and Almond oil.

Another embodiment comprises cyclosporin A, Ufoxane 2®, and Almond oil.

Another embodiment comprises cyclosporin A, a Methyl Ester, and Almond oil.

Another embodiment comprises cyclosporin A, E.B. Cleaner AK®, and Almond oil.

Another embodiment comprises cyclosporin A, Oleocal ME-70®, and Almond oil.

Another embodiment comprises cyclosporin A, Trade Name Company®, and Almond oil.

Another embodiment comprises cyclosporin A, Methyl Esters (cont'd)®, and Almond oil.

Another embodiment comprises cyclosporin A, Oleocal ME-92®, and Almond oil.

Another embodiment comprises cyclosporin A, Oleocal ME-112®, and Almond oil.

Another embodiment comprises cyclosporin A, Oleocal ME-130®, and Almond oil.

Another embodiment comprises cyclosporin A, a Monoglyceride or a Derivative thereof, and Almond oil.

Another embodiment comprises cyclosporin A, Dynacet 211®, and Almond oil.

Another embodiment comprises cyclosporin A, Hetsorb S-20®, and Almond oil.

Another embodiment comprises cyclosporin A, Imwitor 191®, and Almond oil.

Another embodiment comprises cyclosporin A, Imwitor 370®, and Almond oil.

Another embodiment comprises cyclosporin A, Imwitor 375®, and Almond oil.

Another embodiment comprises cyclosporin A, Imwitor 900®, and Almond oil.

Another embodiment comprises cyclosporin A, Imwitor 945®, and Almond oil.

Another embodiment comprises cyclosporin A, Imwitor 2020®, and Almond oil.

Another embodiment comprises cyclosporin A, Kemester 5500®, and Almond oil.

Another embodiment comprises cyclosporin A, Kemester 6000®, and Almond oil.

Another embodiment comprises cyclosporin A, Magrabar GMC®, and Almond oil.

Another embodiment comprises cyclosporin A, Magrabar GMO-CK®, and Almond oil.

Another embodiment comprises cyclosporin A, Magrabar GPC-10®, and Almond oil.

Another embodiment comprises cyclosporin A, Magrabar MDG-5050®, and Almond oil.

Another embodiment comprises cyclosporin A, Monalube 335®, and Almond oil.

Another embodiment comprises cyclosporin A, Monoglycerides Glyceryl Monestearate Archer Daniels Midland Company®, and Almond oil.

Another embodiment comprises cyclosporin A, Rita GMS®, and Almond oil.

Another embodiment comprises cyclosporin A, Ritamulse SCG®, and Almond oil.

Another embodiment comprises cyclosporin A, Softigen 701®, and Almond oil.

Another embodiment comprises cyclosporin A, Tally 100 Plus®, and Almond oil.

Another embodiment comprises cyclosporin A, a Polyethylene Glycol, and Almond oil.

Another embodiment comprises cyclosporin A, Emulgade PL 68/50®, and Almond oil.

Another embodiment comprises cyclosporin A, Lumulse PEG®, and Almond oil.

Another embodiment comprises cyclosporin A, Rhodasurf PEG-400®, and Almond oil.

Another embodiment comprises cyclosporin A, Rhodasurf PEG-600®, and Almond oil.

Another embodiment comprises cyclosporin A, Witconol PEG-400®, and Almond oil.

Another embodiment comprises cyclosporin A, a Polymeric Surfactant, and Almond oil.

Another embodiment comprises cyclosporin A, Acritamer PNC-EG®, and Almond oil.

Another embodiment comprises cyclosporin A, Ag-Rho DEP-775®, and Almond oil.

Another embodiment comprises cyclosporin A, APG 325N Glycoside®, and Almond oil.

Another embodiment comprises cyclosporin A, Aristoflex AVC®, and Almond oil.

Another embodiment comprises cyclosporin A, Aristoflex HMB®, and Almond oil.

Another embodiment comprises cyclosporin A, Burco NPS-225®, and Almond oil.

Another embodiment comprises cyclosporin A, Burco NPS-816®, and Almond oil.

Another embodiment comprises cyclosporin A, Chemccinate 5603®, and Almond oil.

Another embodiment comprises cyclosporin A, Cosmedia Guar C-261 N®, and Almond oil.

Another embodiment comprises cyclosporin A, Gantrez S-95®, and Almond oil.

Another embodiment comprises cyclosporin A, Glucopon 220 UP®, and Almond oil.

Another embodiment comprises cyclosporin A, Glucopon 225 DK®, and Almond oil.

Another embodiment comprises cyclosporin A, Glucopon 425 N®, and Almond oil.

Another embodiment comprises cyclosporin A, Glucopon 600 UP®, and Almond oil.

Another embodiment comprises cyclosporin A, Glucopon 625 UP®, and Almond oil.

Another embodiment comprises cyclosporin A, Pemulen 1621®, and Almond oil.

Another embodiment comprises cyclosporin A, Pemulen 1622®, and Almond oil.

Another embodiment comprises cyclosporin A, Pemulen TR-1®, and Almond oil.

Another embodiment comprises cyclosporin A, Pemulen TR-2®, and Almond oil.

Another embodiment comprises cyclosporin A, Plantacare 818®, and Almond oil.

Another embodiment comprises cyclosporin A, Plantapon LGC Sorb®, and Almond oil.

Another embodiment comprises cyclosporin A, Plantaren 1200N®, and Almond oil.

Another embodiment comprises cyclosporin A, Plantaren 2000N®, and Almond oil.

Another embodiment comprises cyclosporin A, Viscolam AT 64®, and Almond oil.

Another embodiment comprises cyclosporin A, Viscolam AT 64P®, and Almond oil.

Another embodiment comprises cyclosporin A, Viscolam AT 100®, and Almond oil.

Another embodiment comprises cyclosporin A, Viscolam MAC 7®, and Almond oil.

Another embodiment comprises cyclosporin A, Viscolam SMC 20®, and Almond oil.

Another embodiment comprises cyclosporin A, Witbreak RTC-323®, and Almond oil.

Another embodiment comprises cyclosporin A, WSI 3700®, and Almond oil.

Another embodiment comprises cyclosporin A, a Propoxylated or Ethoxylated Fatty Acid, Alcohol, or Alkyl Phenol, and Almond oil.

Another embodiment comprises cyclosporin A, Antarox AA-60®, and Almond oil.

Another embodiment comprises cyclosporin A, Antarox LF-224®, and Almond oil.

Another embodiment comprises cyclosporin A, Burcomul DFE-45®, and Almond oil.

Another embodiment comprises cyclosporin A, Burcoterge LFE-1000®, and Almond oil.

Another embodiment comprises cyclosporin A, Chemal LF-25B®, and Almond oil.

Another embodiment comprises cyclosporin A, Chemal LF-40B®, and Almond oil.

Another embodiment comprises cyclosporin A, Dehypon LS-36®, and Almond oil.

Another embodiment comprises cyclosporin A, Dehypon LS-36®, and Almond oil.

Another embodiment comprises cyclosporin A, Dehypon LS-54®, and Almond oil.

Another embodiment comprises cyclosporin A, Dehypon LS-54®, and Almond oil.

Another embodiment comprises cyclosporin A, Delonic 100 VLF®, and Almond oil.

Another embodiment comprises cyclosporin A, Delonic LF-60 MOD®, and Almond oil.

Another embodiment comprises cyclosporin A, Empiderm B®, and Almond oil.

Another embodiment comprises cyclosporin A, Ethylan 1206®, and Almond oil.

Another embodiment comprises cyclosporin A, Ethylan NS-500K®, and Almond oil.

Another embodiment comprises cyclosporin A, Ethylan NS-500LQ®, and Almond oil.

Another embodiment comprises cyclosporin A, Genapol 1392®, and Almond oil.

Another embodiment comprises cyclosporin A, Genapol 2317®, and Almond oil.

Another embodiment comprises cyclosporin A, Genapol 26EP710®, and Almond oil.

Another embodiment comprises cyclosporin A, Genapol EP 1022®, and Almond oil.

Another embodiment comprises cyclosporin A, Genapol EP 1024®, and Almond oil.

Another embodiment comprises cyclosporin A, Genapol EP 6068®, and Almond oil.

Another embodiment comprises cyclosporin A, Genapol NP915®, and Almond oil.

Another embodiment comprises cyclosporin A, Kieralon MFB®, and Almond oil.

Another embodiment comprises cyclosporin A, Lumisolve CSA-80 V®, and Almond oil.

Another embodiment comprises cyclosporin A, Marlowet 5001®, and Almond oil.

Another embodiment comprises cyclosporin A, Marlox FK 64®, and Almond.oil.

Another embodiment comprises cyclosporin A, Marlox MO 124®, and Almond oil.

Another embodiment comprises cyclosporin A, Marlox S 58®, and Almond oil.

Another embodiment comprises cyclosporin A, Nonatell 1003®, and Almond oil.

Another embodiment comprises cyclosporin A, Nonatell 1038®, and Almond oil.

Another embodiment comprises cyclosporin A, Nonatell 1052®, and Almond oil.

Another embodiment comprises cyclosporin A, Nonatell 1061®, and Almond oil.

Another embodiment comprises cyclosporin A, Nonatell 1075®, and Almond oil.

Another embodiment comprises cyclosporin A, Nonatell 1088®, and Almond oil.

Another embodiment comprises cyclosporin A, Nonatell 1123®, and Almond oil.

Another embodiment comprises cyclosporin A, Nonatell 1153®, and Almond oil.

Another embodiment comprises cyclosporin A, Nonatell 1161®, and Almond oil.

Another embodiment comprises cyclosporin A, Nonatell 1172®, and Almond oil.

Another embodiment comprises cyclosporin A, Nonatell 1181®, and Almond oil.

Another embodiment comprises cyclosporin A, Norfox 36®, and Almond oil.

Another embodiment comprises cyclosporin A, Procetyl AWS®, and Almond oil.

Another embodiment comprises cyclosporin A, Sandoxylate SX 412®, and Almond oil.

Another embodiment comprises cyclosporin A, Sandoxylate SX 418®, and Almond oil.

Another embodiment comprises cyclosporin A, Surfonic JL-80X®, and Almond oil.

Another embodiment comprises cyclosporin A, Surfonic JL-80X-B1®, and Almond oil.

Another embodiment comprises cyclosporin A, Surfonic L4-29X®, and Almond oil.

Another embodiment comprises cyclosporin A, Surfonic LF®, and Almond oil.

Another embodiment comprises cyclosporin A, T-Det A826®, and Almond oil.

Another embodiment comprises cyclosporin A, T-Det LF-416®, and Almond oil.

Another embodiment comprises cyclosporin A, Tergitol Min-Foam 1X®, and Almond oil.

Another embodiment comprises cyclosporin A, Tergitol Min-Foam 2X®, and Almond oil.

Another embodiment comprises cyclosporin A, Triton CF-21®, and Almond oil.

Another embodiment comprises cyclosporin A, Triton CF-76®, and Almond oil.

Another embodiment comprises cyclosporin A, Triton XL-80N®, and Almond oil.

Another embodiment comprises cyclosporin A, Witconbl NS-98®, and Almond oil.

Another embodiment comprises cyclosporin A, Witconol NS-108LQ®, and Almond oil.

Another embodiment comprises cyclosporin A, Witconol NS-145®, and Almond oil.

Another embodiment comprises cyclosporin A, Witconol NS-179®, and Almond oil.

Another embodiment comprises cyclosporin A, a Protein-based Surfactant, and Almond oil.

Another embodiment comprises cyclosporin A, AminoFoam W®, and Almond oil.

Another embodiment comprises cyclosporin A, Amiter LGOD-2®, and Almond oil.

Another embodiment comprises cyclosporin A, Amiter LGS-2®, and Almond oil.

Another embodiment comprises cyclosporin A, Amiter LGS-5®, and Almond oil.

Another embodiment comprises cyclosporin A, Lamepon S®, and Almond oil.

Another embodiment comprises cyclosporin A, Lamepon S®, and Almond oil.

Another embodiment comprises cyclosporin A, Maypon 4C®, and Almond oil.

Another embodiment comprises cyclosporin A, May-Tein C®, and Almond oil.

Another embodiment comprises cyclosporin A, May-Tein CT®, and Almond oil.

Another embodiment comprises cyclosporin A, May-Tein KTS®, and Almond oil.

Another embodiment comprises cyclosporin A, May-Tein SY®, and Almond oil.

Another embodiment comprises cyclosporin A, Plantapon S®, and Almond oil.

Another embodiment comprises cyclosporin A, Proteol APL®, and Almond oil.

Another embodiment comprises cyclosporin A, Proteol OAT®, and Almond oil.

Another embodiment comprises cyclosporin A, Pyroter CPI-40®, and Almond oil.

Another embodiment comprises cyclosporin A, Pyroter GPI-25®, and Almond oil.

Another embodiment comprises cyclosporin A, Supro-Tein S®, and Almond oil.

Another embodiment comprises cyclosporin A, Supro-Tein V®, and Almond oil.

Another embodiment comprises cyclosporin A, a Sarcosine Derivative®, and Almond oil.

Another embodiment comprises cyclosporin A, Crodasinic LS-30®, and Almond oil.

Another embodiment comprises cyclosporin A, Vanseal CS®, and Almond oil.

Another embodiment comprises cyclosporin A, Vanseal LS®, and Almond oil.

Another embodiment comprises cyclosporin A, Vanseal MS®, and Almond oil.

Another embodiment comprises cyclosporin A, Vanseal NACS-30®, and Almond oil.

Another embodiment comprises cyclosporin A, Vanseal NALS-95®, and Almond oil.

Another embodiment comprises cyclosporin A, Vanseal OS®, and Almond oil.

Another embodiment comprises cyclosporin A,® and Almond oil.

Another embodiment comprises cyclosporin A, a Silicone-based Surfactant®, and Almond oil.

Another embodiment comprises cyclosporin A, Abil-B-9950®, and Almond oil.

Another embodiment comprises cyclosporin A, Abil Care 85®, and Almond oil.

Another embodiment comprises cyclosporin A, Abil EM 90®, and Almond oil.

Another embodiment comprises cyclosporin A, Abil EM 97®, and Almond oil.

Another embodiment comprises cyclosporin A, Abil WE-09®, and Almond oil.

Another embodiment comprises cyclosporin A, Dow Corning 1248 Fluid®, and Almond oil.

Another embodiment comprises cyclosporin A, Dow Corning 3225C®, and Almond oil.

Another embodiment comprises cyclosporin A, Dow Corning 5200®, and Almond oil.

Another embodiment comprises cyclosporin A, Dow Corning Q4-3667®, and Almond oil.

Another embodiment comprises cyclosporin A, Monasil PCA®, and Almond oil.

Another embodiment comprises cyclosporin A, Monasil PDM®, and Almond oil.

Another embodiment comprises cyclosporin A, Monasil PLN®, and Almond oil.

Another embodiment comprises cyclosporin A, Polyderm PPI-SI-WS®, and Almond oil.

Another embodiment comprises cyclosporin A, Troysol 380W®, and Almond oil.

Another embodiment comprises cyclosporin A, Troysol S366®, and Almond oil.

Another embodiment comprises cyclosporin A, a Sorbitan Derivative, and Almond oil.

Another embodiment comprises cyclosporin A, Alkamuls SML®, and Almond oil.

Another embodiment comprises cyclosporin A, Alkamuls SMO®, and Almond oil.

Another embodiment comprises cyclosporin A, Alkamuls STO®, and Almond oil.

Another embodiment comprises cyclosporin A, Arlacel 20®, and Almond oil.

Another embodiment comprises cyclosporin A, Arlacel 40®, and Almond oil.

Another embodiment comprises cyclosporin A, Arlacel 60®, and Almond oil.

Another embodiment comprises cyclosporin A, Arlacel 80®, and Almond oil.

Another embodiment comprises cyclosporin A, Arlacel C®, and Almond oil.

Another embodiment comprises cyclosporin A, Armul 21®, and Almond oil.

Another embodiment comprises cyclosporin A, Atlox 80®, and Almond oil.

Another embodiment comprises cyclosporin A, Atlox 847®, and Almond oil.

Another embodiment comprises cyclosporin A, Atlox 1045A®, and Almond oil.

Another embodiment comprises cyclosporin A, Canarcel 20®, and Almond oil.

Another embodiment comprises cyclosporin A, Canarcel 60®, and Almond oil.

Another embodiment comprises cyclosporin A, Canarcel 80®, and Almond oil.

Another embodiment comprises cyclosporin A, Canarcel TW 20®, and Almond oil.

Another embodiment comprises cyclosporin A, Canarcel TW 60®, and Almond oil.

Another embodiment comprises cyclosporin A, Canarcel TW 80®, and Almond oil.

Another embodiment comprises cyclosporin A, Coladet BSB-P®, and Almond oil.

Another embodiment comprises cyclosporin A, Customulse O-20®, and Almond oil.

Another embodiment comprises cyclosporin A, Dehymuls E®, and Almond oil.

Another embodiment comprises cyclosporin A, DeSotan SMO®, and Almond oil.

Another embodiment comprises cyclosporin A, DeSotan SMO-20®, and Almond oil.

Another embodiment comprises cyclosporin A, DeSotan SMT®, and Almond oil.

Another embodiment comprises cyclosporin A, DeSotan SMT-20®, and Almond oil.

Another embodiment comprises cyclosporin A, Durfax 60®, and Almond oil.

Another embodiment comprises cyclosporin A, Durfax 65®, and Almond oil.

Another embodiment comprises cyclosporin A, Durfax 80®, and Almond oil.

Another embodiment comprises cyclosporin A, Durtan 60®, and Almond oil.

Another embodiment comprises cyclosporin A, Durtan 65®, and Almond oil.

Another embodiment comprises cyclosporin A, Liposorb L®, and Almond oil.

Another embodiment comprises cyclosporin A, Liposorb L-10®, and Almond oil.

Another embodiment comprises cyclosporin A, Liposorb L-20®, and Almond oil.

Another embodiment comprises cyclosporin A, Liposorb O®, and Almond oil.

Another embodiment comprises cyclosporin A, Liposorb O-20®, and Almond oil.

Another embodiment comprises cyclosporin A, Liposorb P®, and Almond oil.

Another embodiment comprises cyclosporin A, Liposorb P-20®, and Almond oil.

Another embodiment comprises cyclosporin A, Liposorb S®, and Almond oil.

Another embodiment comprises cyclosporin A, Liposorb S-20®, and Almond oil.

Another embodiment comprises cyclosporin A, Liposorb SQO®, and Almond oil.

Another embodiment comprises cyclosporin A, Liposorb TO®, and Almond oil.

Another embodiment comprises cyclosporin A, Liposorb TS®, and Almond oil.

Another embodiment comprises cyclosporin A, Liposorb TS-20®, and Almond oil.

Another embodiment comprises cyclosporin A, Lumisorb PS®, and Almond oil.

Another embodiment comprises cyclosporin A, Lumisorb SMO (T)®, and Almond oil.

Another embodiment comprises cyclosporin A, Lumisorb SMS K®, and Almond oil.

Another embodiment comprises cyclosporin A, Lumisorb SSO®, and Almond oil.

Another embodiment comprises cyclosporin A, Lumisorb STS K®, and Almond oil.

Another embodiment comprises cyclosporin A, Lumisorb STT®, and Almond oil.

Another embodiment comprises cyclosporin A, Magrabar SMO®, and Almond oil.

Another embodiment comprises cyclosporin A, Magrabar SMO-VEG®, and Almond oil.

Another embodiment comprises cyclosporin A, Magrabar SMT®, and Almond oil.

Another embodiment comprises cyclosporin A, Magrabar STO®, and Almond oil.

Another embodiment comprises cyclosporin A, Miracare BC-27®, and Almond oil.

Another embodiment comprises cyclosporin A, Ritabate 20®, and Almond oil.

Another embodiment comprises cyclosporin A, Ritabate 40®, and Almond oil.

Another embodiment comprises cyclosporin A, Ritabate 60®, and Almond oil.

Another embodiment comprises cyclosporin A, Ritabate 80®, and Almond oil.

Another embodiment comprises cyclosporin A, T-Maz®, and Almond oil.

Another embodiment comprises cyclosporin A, Tego SML®, and Almond oil.

Another embodiment comprises cyclosporin A, Tego SML 20®, and Almond oil.

Another embodiment comprises cyclosporin A, Tego SMO 80 V®, and Almond oil.

Another embodiment comprises cyclosporin A, Tego SMO V®, and Almond oil.

Another embodiment comprises cyclosporin A, Tego SMS®, and Almond oil.

Another embodiment comprises cyclosporin A, Tego STO V®, and Almond oil.

Another embodiment comprises cyclosporin A, Tween 21®, and Almond oil.

Another embodiment comprises cyclosporin A, Tween 40®, and Almond oil.

Another embodiment comprises cyclosporin A, Tween 60®, and Almond oil.

Another embodiment comprises cyclosporin A, Tween 60 K®, and Almond oil.

Another embodiment comprises cyclosporin A, Tween 61®, and Almond oil.

Another embodiment comprises cyclosporin A, Tween 65®, and Almond oil.

Another embodiment comprises cyclosporin A, Tween 80®, and Almond oil.

Another embodiment comprises cyclosporin A, Tween 80 K®, and Almond oil.

Another embodiment comprises cyclosporin A, Tween 81®, and Almond oil.

Another embodiment comprises cyclosporin A, Tween 85®, and Almond oil.

Another embodiment comprises cyclosporin A, a Sucrose or Glucose Ester, or Derivative thereof, and Almond oil.

Another embodiment comprises cyclosporin A, DeSuff GOS-P-60WCG®, and Almond oil.

Another embodiment comprises cyclosporin A, Glucam E-20 Distearate®, and Almond oil.

Another embodiment comprises cyclosporin A, Glucamate DOE-120®, and Almond oil.

Another embodiment comprises cyclosporin A, Glucamate SSE-20®, and Almond oil.

Another embodiment comprises cyclosporin A, Glucate DO®, and Almond oil.

Another embodiment comprises cyclosporin A, Glucate SS®, and Almond oil.

Another embodiment comprises cyclosporin A, Glucopon 425 UP®, and Almond oil.

Another embodiment comprises cyclosporin A, Isolan IS®, and Almond oil.

Another embodiment comprises cyclosporin A, Mazon 40®, and Almond oil.

Another embodiment comprises cyclosporin A, Montanov 82®, and Almond oil.

Another embodiment comprises cyclosporin A, Montanov 202®, and Almond oil.

Another embodiment comprises cyclosporin A, Montanov S®, and Almond oil.

Another embodiment comprises cyclosporin A, Rheozan®, and Almond oil.

Another embodiment comprises cyclosporin A, Simulsol AS 48®, and Almond oil.

Another embodiment comprises cyclosporin A, Simulsol SL 4®, and Almond oil.

Another embodiment comprises cyclosporin A, Simulsol SL 10®, and Almond oil.

Another embodiment comprises cyclosporin A, Simulsol SL 11W®, and Almond oil.

Another embodiment comprises cyclosporin A, Simulsol SL 55®, and Almond oil.

Another embodiment comprises cyclosporin A, Suga Nate 100 and 160®, and Almond oil.

Another embodiment comprises cyclosporin A, Tego Care 450®, and Almond oil.

Another embodiment comprises cyclosporin A, Tego Care CG 90®, and Almond oil.

Another embodiment comprises cyclosporin A, Tego Care PS®, and Almond oil.

Another embodiment comprises cyclosporin A, Tegosoft PSE 141 G®, and Almond oil.

Another embodiment comprises cyclosporin A, Tegotens G 826®, and Almond oil.

Another embodiment comprises cyclosporin A, Triton BG-10 (70%)®, and Almond oil.

Another embodiment comprises cyclosporin A, Triton CG-110 (60%)®, and Almond oil.

Another embodiment comprises cyclosporin A, Wickenol 545®, and Almond oil.

Another embodiment comprises cyclosporin A, an alcohol, and Corn oil.

Another embodiment comprises cyclosporin A, Diglycerol®, and Corn oil.

Another embodiment comprises cyclosporin A, Hetoxide GT-80®, and Corn oil.

Another embodiment comprises cyclosporin A, Lexemul BEO®, and Corn oil.

Another embodiment comprises cyclosporin A, Polyglycerol-3®, and Corn oil.

Another embodiment comprises cyclosporin A, Redicote E Series®, and Corn oil.

Another embodiment comprises cyclosporin A, Simulsol OX 1005L®, and Corn oil.

Another embodiment comprises cyclosporin A, Stanfax 567®, and Corn oil.

Another embodiment comprises cyclosporin A, TA-1618®, and Corn oil.

Another embodiment comprises cyclosporin A, Witconol H-31 A®, and Corn oil.

Another embodiment comprises cyclosporin A, an amine oxide, and Corn oil.

Another embodiment comprises cyclosporin A, AO-405®, and Corn oil.

Another embodiment comprises cyclosporin A, AO-455®, and Corn oil.

Another embodiment comprises cyclosporin A, AO 728 Special®, and Corn oil.

Another embodiment comprises cyclosporin A, Barlox 12®, and Corn oil.

Another embodiment comprises cyclosporin A, Barlox 14®, and Corn oil.

Another embodiment comprises cyclosporin A, Burcoxide Lo®, and Corn oil.

Another embodiment comprises cyclosporin A, Caloxamine LO®, and Corn oil.

Another embodiment comprises cyclosporin A, Chemoxide CAW®, and Corn oil.

Another embodiment comprises cyclosporin A, Chemoxide LM-30®, and Corn oil.

Another embodiment comprises cyclosporin A, Chemoxide LO®, and Corn oil.

Another embodiment comprises cyclosporin A, Chemoxide MO®, and Corn oil.

Another embodiment comprises cyclosporin A, Colalux CAO-35®, and Corn oil.

Another embodiment comprises cyclosporin A, Colalux LO®, and Corn oil.

Another embodiment comprises cyclosporin A, DeMox CAPO®, and Corn oil.

Another embodiment comprises cyclosporin A, DeMox CSG-30®, and Corn oil.

Another embodiment comprises cyclosporin A, DeMox LAO®, and Corn oil.

Another embodiment comprises cyclosporin A, Emcol L®, and Corn oil.

Another embodiment comprises cyclosporin A, Empigen OB®, and Corn oil.

Another embodiment comprises cyclosporin A, Empigen OS/A®, and Corn oil.

Another embodiment comprises cyclosporin A, Foamox CDO®, and Corn oil.

Another embodiment comprises cyclosporin A, Foamox DMM®, and Corn oil.

Another embodiment comprises cyclosporin A, Foamox DMS®, and Corn oil.

Another embodiment comprises cyclosporin A, Genaminox KC®, and Corn oil.

Another embodiment comprises cyclosporin A, Genaminox LA®, and Corn oil.

Another embodiment comprises cyclosporin A, Hartofoam SAO®, and Corn oil.

Another embodiment comprises cyclosporin A, Hartox DMCD®, and Corn oil.

Another embodiment comprises cyclosporin A, Lipowax DAT®, and Corn oil.

Another embodiment comprises cyclosporin A, Lipowax PB Pastilles®, and Corn oil.

Another embodiment comprises cyclosporin A, Mackamine C8®, and Corn oil.

Another embodiment comprises cyclosporin A, Mackamine C10®, and Corn oil.

Another embodiment comprises cyclosporin A, Mackamine C14®, and Corn oil.

Another embodiment comprises cyclosporin A, Mackamine CAO®, and Corn oil.

Another embodiment comprises cyclosporin A, Mackamine CO®, and Corn oil.

Another embodiment comprises cyclosporin A, Mackamine LO®, and Corn oil.

Another embodiment comprises cyclosporin A, Mackamine O2®, and Corn oil.

Another embodiment comprises cyclosporin A, Mackamine SAO®, and Corn oil.

Another embodiment comprises cyclosporin A, Mackamine SO®, and Corn oil.

Another embodiment comprises cyclosporin A, Mazox KCAO®, and Corn oil.

Another embodiment comprises cyclosporin A, Monalac MO®, and Corn oil.

Another embodiment comprises cyclosporin A, Norfox LDA®, and Corn oil.

Another embodiment comprises cyclosporin A, Rhodamox LO®, and Corn oil.

Another embodiment comprises cyclosporin A, Schercamox C-AA®, and Corn oil.

Another embodiment comprises cyclosporin A, Schercamox DMA®, and Corn oil.

Another embodiment comprises cyclosporin A, Schercamox DML®, and Corn oil.

Another embodiment comprises cyclosporin A, Schercamox DMM®, and Corn oil.

Another embodiment comprises cyclosporin A, Schercamox DMS®, and Corn oil.

Another embodiment comprises cyclosporin A, Tegotens DO®, and Corn oil.

Another embodiment comprises cyclosporin A, Tomah AO-14-2®, and Corn oil.

Another embodiment comprises cyclosporin A, Triaminox CDO®, and Corn oil.

Another embodiment comprises cyclosporin A, a block polymer, and Corn oil.

Another embodiment comprises cyclosporin A, AL 2070®, and Corn oil.

Another embodiment comprises cyclosporin A, Antarox 17-R-2®, and Corn oil.

Another embodiment comprises cyclosporin A, Antarox 25-R-2®, and Corn oil.

Another embodiment comprises cyclosporin A, Antarox 31-R-1®, and Corn oil.

Another embodiment comprises cyclosporin A, Antarox P-84®, and Corn oil.

Another embodiment comprises cyclosporin A, Antarox P-104/H®, and Corn oil.

Another embodiment comprises cyclosporin A, Arnox BP-Series®, and Corn oil.

Another embodiment comprises cyclosporin A, Chemonic 435®, and Corn oil.

Another embodiment comprises cyclosporin A, Chemonic D-25®, and Corn oil.

Another embodiment comprises cyclosporin A, Chemonic PL Series®, and Corn oil.

Another embodiment comprises cyclosporin A, Ethox L-121®, and Corn oil.

Another embodiment comprises cyclosporin A, Ethox L-122®, and Corn oil.

Another embodiment comprises cyclosporin A, Genapol PF-10®, and Corn oil.

Another embodiment comprises cyclosporin A, Genapol PF-20®, and Corn oil.

Another embodiment comprises cyclosporin A, Genapol PF-40A®, and Corn oil.

Another embodiment comprises cyclosporin A, Norfox 2-LF®, and Corn oil.

Another embodiment comprises cyclosporin A, Simulsol NW 342®, and Corn oil.

Another embodiment comprises cyclosporin A, T-Det BP-1®, and Corn oil.

Another embodiment comprises cyclosporin A, T-Det XD®, and Corn oil.

Another embodiment comprises cyclosporin A, T-Det XH®, and Corn oil.

Another embodiment comprises cyclosporin A, Triton CF-32®, and Corn oil.

Another embodiment comprises cyclosporin A, Witconol 171®, and Corn oil.

Another embodiment comprises cyclosporin A, Witconol 324®, and Corn oil.

Another embodiment comprises cyclosporin A, Witconol 324D®, and Corn oil.

Another embodiment comprises cyclosporin A, Witconol PD-2000®, and Corn oil.

Another embodiment comprises cyclosporin A, a Carboxylated Alcohol or Alkylphenol Ethoxylate, and Corn oil.

Another embodiment comprises cyclosporin A, Emcol CN-6®, and Corn oil.

Another embodiment comprises cyclosporin A, Ethcarb®, and Corn oil.

Another embodiment comprises cyclosporin A, Gemtex WNT-Conc®, and Corn oil.

Another embodiment comprises cyclosporin A, Incrodet TD7-C®, and Corn oil.

Another embodiment comprises cyclosporin A, Marlinat CM 105/80®, and Corn oil.

Another embodiment comprises cyclosporin A, Marlowet 1072®, and Corn oil.

Another embodiment comprises cyclosporin A, Marlowet 4530®, and Corn oil.

Another embodiment comprises cyclosporin A, Marlowet 4530 LF®, and Corn oil.

Another embodiment comprises cyclosporin A, Marlowet 4534®, and Corn oil.

Another embodiment comprises cyclosporin A, Marlowet 4538®, and Corn oil.

Another embodiment comprises cyclosporin A, Marlowet 4539®, and Corn oil.

Another embodiment comprises cyclosporin A, Marlowet 4539 LF®, and Corn oil.

Another embodiment comprises cyclosporin A, Marlowet 4541®, and Corn oil.

Another embodiment comprises cyclosporin A, Miranate LEC-80®, and Corn oil.

Another embodiment comprises cyclosporin A, Sandopan B®, and Corn oil.

Another embodiment comprises cyclosporin A, Sandopan B Modified®, and Corn oil.

Another embodiment comprises cyclosporin A, Sandopan LS-24 Gel®, and Corn oil.

Another embodiment comprises cyclosporin A, Surfine T-A®, and Corn oil.

Another embodiment comprises cyclosporin A, a Carboxylic Acid/Fatty Acid, and Corn oil.

Another embodiment comprises cyclosporin A, Colaterge RAM®, and Corn oil.

Another embodiment comprises cyclosporin A, Colatrope INC®, and Corn oil.

Another embodiment comprises cyclosporin A, Crodacid B®, and Corn oil.

Another embodiment comprises cyclosporin A, DeTrope CA-100®, and Corn oil.

Another embodiment comprises cyclosporin A, Latol MTO®, and Corn oil.

Another embodiment comprises cyclosporin A, Lumulse CC-33 K®, and Corn oil.

Another embodiment comprises cyclosporin A, Mulls 2218®, and Corn oil.

Another embodiment comprises cyclosporin A, OL-600®, and Corn oil.

Another embodiment comprises cyclosporin A, OL-800®, and Corn oil.

Another embodiment comprises cyclosporin A, R-910®, and Corn oil.

Another embodiment comprises cyclosporin A, S-210®, and Corn oil.

Another embodiment comprises cyclosporin A, Sandopan DTC Acid®, and Corn oil.

Another embodiment comprises cyclosporin A, Sandopan LS 24 N®, and Corn oil.

Another embodiment comprises cyclosporin A, Sandopan MA-18®, and Corn oil.

Another embodiment comprises cyclosporin A, an Ethoxylated Alcohol, and Corn oil.

Another embodiment comprises cyclosporin A, Adsee 799®, and Corn oil.

Another embodiment comprises cyclosporin A, Adsee 799®, and Corn oil.

Another embodiment comprises cyclosporin A, Alfonic 610-3.5®, and Corn oil.

Another embodiment comprises cyclosporin A, Alfonic 810-2®, and Corn oil.

Another embodiment comprises cyclosporin A, Alfonic 810-6®, and Corn oil.

Another embodiment comprises cyclosporin A, Alfonic 1012-3®, and Corn oil.

Another embodiment comprises cyclosporin A, Alfonic 1012-5®, and Corn oil.

Another embodiment comprises cyclosporin A, Alfonic 1216CO-1.5®, and Corn oil.

Another embodiment comprises cyclosporin A, Aifonic 1216CO-7®, and Corn oil.

Another embodiment comprises cyclosporin A, Alfonic 1412-3®, and Corn oil.

Another embodiment comprises cyclosporin A, Alfonic 1412-7®, and Corn oil.

Another embodiment comprises cyclosporin A, Arlasolve 200®, and Corn oil.

Another embodiment comprises cyclosporin A, Arlasolve 200 Liquid®, and Corn oil.

Another embodiment comprises cyclosporin A, Armix 180-C®, and Corn oil.

Another embodiment comprises cyclosporin A, Armix 183®, and Corn oil.

Another embodiment comprises cyclosporin A, Armul 2404®, and Corn oil.

Another embodiment comprises cyclosporin A, Armul 2404®, and Corn oil.

Another embodiment comprises cyclosporin A, Atlas EMJ-C®, and Corn oil.

Another embodiment comprises cyclosporin A, Atlas G-2109®, and Corn oil.

Another embodiment comprises cyclosporin A, Atlas G-3886®, and Corn oil.

Another embodiment comprises cyclosporin A, Atlas G-3890®, and Corn oil.

Another embodiment comprises cyclosporin A, Bio Soft E-200®, and Corn oil.

Another embodiment comprises cyclosporin A, Bio Soft E-300®, and Corn oil.

Another embodiment comprises cyclosporin A, Bio Soft E-400®, and Corn oil.

Another embodiment comprises cyclosporin A, Bio Soft EN 600®, and Corn oil.

Another embodiment comprises cyclosporin A, Bio Soft TD-400®, and Corn oil.

Another embodiment comprises cyclosporin A, Bio Soft TD-630®, and Corn oil.

Another embodiment comprises cyclosporin A, Brij 30®, and Corn oil.

Another embodiment comprises cyclosporin A, Brij 52®, and Corn oil.

Another embodiment comprises cyclosporin A, Brij 56®, and Corn oil.

Another embodiment comprises cyclosporin A, Brij 58®, and Corn oil.

Another embodiment comprises cyclosporin A, Brij 72®, and Corn oil.

Another embodiment comprises cyclosporin A, Brij 76®, and Corn oil.

Another embodiment comprises cyclosporin A, Brij 78®, and Corn oil.

Another embodiment comprises cyclosporin A, Brij 93®, and Corn oil.

Another embodiment comprises cyclosporin A, Brij 97®, and Corn oil.

Another embodiment comprises cyclosporin A, Brij 98®, and Corn oil.

Another embodiment comprises cyclosporin A, Brij 700®, and Corn oil.

Another embodiment comprises cyclosporin A, Brij 700 S®, and Corn oil.

Another embodiment comprises cyclosporin A, Brij 721®, and Corn oil.

Another embodiment comprises cyclosporin A, Brij 721 S®, and Corn oil.

Another embodiment comprises cyclosporin A, Burcoterge CDG®, and Corn oil.

Another embodiment comprises cyclosporin A, Canasol AT 600®, and Corn oil.

Another embodiment comprises cyclosporin A, Canasol AT 800®, and Corn oil.

Another embodiment comprises cyclosporin A, Canasol AT 1200®, and Corn oil.

Another embodiment comprises cyclosporin A, Canasol BJ 35®, and Corn oil.

Another embodiment comprises cyclosporin A, Canasol BJ 36®, and Corn oil.

Another embodiment comprises cyclosporin A, Canasol EJ 52®, and Corn oil.

Another embodiment comprises cyclosporin A, Canasol BJ 58®, and Corn oil.

Another embodiment comprises cyclosporin A, Canasol BJ 72®, and Corn oil.

Another embodiment comprises cyclosporin A, Canasol BJ 78®, and Corn oil.

Another embodiment comprises cyclosporin A, Canasol EJ 98®, and Corn oil.

Another embodiment comprises cyclosporin A, Canasol BJ 307®, and Corn oil.

Another embodiment comprises cyclosporin A, Cerfak 1400®, and Corn oil.

Another embodiment comprises cyclosporin A, Cetomacrogol 1000 BP®, and Corn oil.

Another embodiment comprises cyclosporin A, Chemonic C-2®, and Corn oil.

Another embodiment comprises cyclosporin A, Chemonic C-10®, and Corn oil.

Another embodiment comprises cyclosporin A, Chemonic C-20®, and Corn oil.

Another embodiment comprises cyclosporin A, Chemonic CT-12®, and Corn oil.

Another embodiment comprises cyclosporin A, Chemonic CT-20®, and Corn oil.

Another embodiment comprises cyclosporin A, Chemonic CT-30®, and Corn oil.

Another embodiment comprises cyclosporin A, Chemonic CT-55®, and Corn oil.

Another embodiment comprises cyclosporin A, Chemonic G-7®, and Corn oil.

Another embodiment comprises cyclosporin A, Chemdnic G-26®, and Corn oil.

Another embodiment comprises cyclosporin A, Chemonic L-4®, and Corn oil.

Another embodiment comprises cyclosporin A, Chemonic L-7®, and Corn oil.

Another embodiment comprises cyclosporin A, Chemonic L-12®, and Corn oil.

Another embodiment comprises cyclosporin A, Chemonic L-23®, and Corn oil.

Another embodiment comprises cyclosporin A, Chemonic O-2®, and Corn oil.

Another embodiment comprises cyclosporin A, Chemonic O-5®, and Corn oil.

Another embodiment comprises cyclosporin A, Chemonic O-10®, and Corn oil.

Another embodiment comprises cyclosporin A, Chemonic O-20®, and Corn oil.

Another embodiment comprises cyclosporin A, Chemonic S-2®, and Corn oil.

Another embodiment comprises cyclosporin A, Chemonic S-10®, and Corn oil.

Another embodiment comprises cyclosporin A, Chemonic S-20®, and Corn oil.

Another embodiment comprises cyclosporin A, Colamulse FE®, and Corn oil.

Another embodiment comprises cyclosporin A, Cremophor A 20®, and Corn oil.

Another embodiment comprises cyclosporin A, Cremophor SA 2®, and Corn oil.

Another embodiment comprises cyclosporin A, Dehydol 100®, and Corn oil.

Another embodiment comprises cyclosporin A, Dehydol O-4®, and Corn oil.

Another embodiment comprises cyclosporin A, Delonic C-18®, and Corn oil.

Another embodiment comprises cyclosporin A, DeSonic 6T®, and Corn oil.

Another embodiment comprises cyclosporin A, DeSonic 9D®, and Corn oil.

Another embodiment comprises cyclosporin A, DeSonic 9T®, and Corn oil.

Another embodiment comprises cyclosporin A, DeSonic 12D®, and Corn oil.

Another embodiment comprises cyclosporin A, DeSonic 12T®, and Corn oil.

Another embodiment comprises cyclosporin A, DeSonic 15T®, and Corn oil.

Another embodiment comprises cyclosporin A, DeSonic TDA-9®, and Corn oil.

Another embodiment comprises cyclosporin A, DeThox GLG-7®, and Corn oil.

Another embodiment comprises cyclosporin A, DeThox GLG-26®, and Corn oil.

Another embodiment comprises cyclosporin A, DeThox LA-4®, and Corn oil.

Another embodiment comprises cyclosporin A, DeThox LA-23®, and Corn oil.

Another embodiment comprises cyclosporin A, DeThox SA-80®, and Corn oil.

Another embodiment comprises cyclosporin A, Disponil O5®, and Corn oil.

Another embodiment comprises cyclosporin A, Eccoterge EO-41B®, and Corn oil.

Another embodiment comprises cyclosporin A, Empilan KA2.5/90®, and Corn oil.

Another embodiment comprises cyclosporin A, Empilan KA5/90®, and Corn oil.

Another embodiment comprises cyclosporin A, Empilan KM-20®, and Corn oil.

Another embodiment comprises cyclosporin A, Empilan KM-50®, and Corn oil.

Another embodiment comprises cyclosporin A, Empilan L-23®, and Corn oil.

Another embodiment comprises cyclosporin A, Ethylan 25-3®, and Corn oil.

Another embodiment comprises cyclosporin A, Ethylan 1204®, and Corn oil.

Another embodiment comprises cyclosporin A, Ethylan DA-4®, and Corn oil.

Another embodiment comprises cyclosporin A, Ethylan LA-230®, and Corn oil.

Another embodiment comprises cyclosporin A, Ethylan SN®, and Corn oil.

Another embodiment comprises cyclosporin A, Ethylan TD-60®, and Corn oil.

Another embodiment comprises cyclosporin A, Ethylan TD-100®, and Corn oil.

Another embodiment comprises cyclosporin A, Ethylan TD-1407®, and Corn oil.

Another embodiment comprises cyclosporin A, Eumulgin B1®, and Corn oil.

Another embodiment comprises cyclosporin A, Eumulgin B2®, and Corn oil.

Another embodiment comprises cyclosporin A, Eumulgin B3®, and Corn oil.

Another embodiment comprises cyclosporin A, Eumulgin O-10®, and Corn oil.

Another embodiment comprises cyclosporin A, Flo Mo 80/20®, and Corn oil.

Another embodiment comprises cyclosporin A, Flo Mo Low Foam®, and Corn oil.

Another embodiment comprises cyclosporin A, Forlan C-24®, and Corn oil.

Another embodiment comprises cyclosporin A, Genapol 1454®, and Corn oil.

Another embodiment comprises cyclosporin A, Genapol BA-020®, and Corn oil.

Another embodiment comprises cyclosporin A, Genapol BA-040®, and Corn oil.

Another embodiment comprises cyclosporin A, Genapol C-100®, and Corn oil.

Another embodiment comprises cyclosporin A, Genapol DA 060®, and Corn oil.

Another embodiment comprises cyclosporin A, Genapol HS 020®, and Corn oil.

Another embodiment comprises cyclosporin A, Genapol HS 200®, and Corn oil.

Another embodiment comprises cyclosporin A, Genapol ID-040®, and Corn oil.

Another embodiment comprises cyclosporin A, Genapol ID-060®, and Corn oil.

Another embodiment comprises cyclosporin A, Genapol ID-090®, and Corn oil.

Another embodiment comprises cyclosporin A, Genapol LA 010®, and Corn oil.

Another embodiment comprises cyclosporin A, Genapol LA 020®, and Corn oil.

Another embodiment comprises cyclosporin A, Genapol LA 030®, and Corn oil.

Another embodiment comprises cyclosporin A, Genapol LA 040®, and Corn oil.

Another embodiment comprises cyclosporin A, Genapol LA 050®, and Corn oil.

Another embodiment comprises cyclosporin A, Genapol LA 060®, and Corn oil.

Another embodiment comprises cyclosporin A, Genapol LA 070®, and Corn oil.

Another embodiment comprises cyclosporin A, Genapol LA 070S®, and Corn oil.

Another embodiment comprises cyclosporin A, Genapol LA 230®, and Corn oil.

Another embodiment comprises cyclosporin A, Genapol O 020®, and Corn oil.

Another embodiment comprises cyclosporin A, Genapol O 050®, and Corn oil.

Another embodiment comprises cyclosporin A, Genapol O 100®, and Corn oil.

Another embodiment comprises cyclosporin A, Genapol O 200®, and Corn oil.

Another embodiment comprises cyclosporin A, Genapol SA 030®, and Corn oil.

Another embodiment comprises cyclosporin A, Genapol SA 120®, and Corn oil.

Another embodiment comprises cyclosporin A, Genapol T-020®, and Corn oil.

Another embodiment comprises cyclosporin A, Genapol UD-030®, and Corn oil.

Another embodiment comprises cyclosporin A, Genapol UD-050®, and Corn oil.

Another embodiment comprises cyclosporin A, Genapol UD-070®, and Corn oil.

Another embodiment comprises cyclosporin A, Genapol UD-079®, and Corn oil.

Another embodiment comprises cyclosporin A, Genapol UD-080®, and Corn oil.

Another embodiment comprises cyclosporin A, Genapol UD-110®, and Corn oil.

Another embodiment comprises cyclosporin A, Genapol X 030®, and Corn oil.

Another embodiment comprises cyclosporin A, Genapol X 050®, and Corn oil.

Another embodiment comprises cyclosporin A, Genapol X 060®, and Corn oil.

Another embodiment comprises cyclosporin A, Genapol X 070®, and Corn oil.

Another embodiment comprises cyclosporin A, Genapol X 080®, and Corn oil.

Another embodiment comprises cyclosporin A, Genapol X 100®, and Corn oil.

Another embodiment comprises cyclosporin A, Genapol X159®, and Corn oil.

Another embodiment comprises cyclosporin A, Generol 122 E5®, and Corn oil.

Another embodiment comprises cyclosporin A, Generol 122 E25®, and Corn oil.

Another embodiment comprises cyclosporin A, Hostacerin T-3®, and Corn oil.

Another embodiment comprises cyclosporin A, Iconol LF 110®, and Corn oil.

Another embodiment comprises cyclosporin A, Incropol CS-20®, and Corn oil.

Another embodiment comprises cyclosporin A, Lexemul CS-20®, and Corn oil.

Another embodiment comprises cyclosporin A, Liponic EG-1®, and Corn oil.

Another embodiment comprises cyclosporin A, Lipowax D®, and Corn oil.

Another embodiment comprises cyclosporin A, Lipowax G®, and Corn oil.

Another embodiment comprises cyclosporin A, Lipowax NI®, and Corn oil.

Another embodiment comprises cyclosporin A, Lipowax P®, and Corn oil.

Another embodiment comprises cyclosporin A, Lipowax P-31®, and Corn oil.

Another embodiment comprises cyclosporin A, Lipowax PR®, and Corn oil.

Another embodiment comprises cyclosporin A, Lumulse CS-20®, and Corn oil.

Another embodiment comprises cyclosporin A, Macol CSA-20®, and Corn oil.

Another embodiment comprises cyclosporin A, Marlox B 24/50®, and Corn oil.

Another embodiment comprises cyclosporin A, Mazawet 77®, and Corn oil.

Another embodiment comprises cyclosporin A, Norfox 1713®, and Corn oil.

Another embodiment comprises cyclosporin A, Norfox 2579®, and Corn oil.

Another embodiment comprises cyclosporin A, Norfox Lo Foam®, and Corn oil.

Another embodiment comprises cyclosporin A, Promulgen D®, and Corn oil.

Another embodiment comprises cyclosporin A, Promulgen G®, and Corn oil.

Another embodiment comprises cyclosporin A, Renex 30®, and Corn oil.

Another embodiment comprises cyclosporin A, Renex 36®, and Corn oil.

Another embodiment comprises cyclosporin A, Rhodasurf A 24®, and Corn oil.

Another embodiment comprises cyclosporin A, Rhodasurf AAE-10®, and Corn oil.

Another embodiment comprises cyclosporin A, Rhodasurf BEH-25®, and Corn oil.

Another embodiment comprises cyclosporin A, Rhodasurf BEH-40®, and Corn oil.

Another embodiment comprises cyclosporin A, Rhodasurf DA 530®, and Corn oil.

Another embodiment comprises cyclosporin A, Rhodasurf DA 630®, and Corn oil.

Another embodiment comprises cyclosporin A, Rhodasurf DA 639®, and Corn oil.

Another embodiment comprises cyclosporin A, Rhodasurf LAN-23®, and Corn oil.

Another embodiment comprises cyclosporin A, Rhodasurf ON-870®, and Corn oil.

Another embodiment comprises cyclosporin A, Rhodasurf ON-877®, and Corn oil.

Another embodiment comprises cyclosporin A, Rhodasurf TB-970 FLK®, and Corn oil.

Another embodiment comprises cyclosporin A, Ritacet-20®, and Corn oil.

Another embodiment comprises cyclosporin A, Ritachol 1000®, and Corn oil.

Another embodiment comprises cyclosporin A, Ritachol 2000®, and Corn oil.

Another embodiment comprises cyclosporin A, Ritachol 5000®, and Corn oil.

Another embodiment comprises cyclosporin A, Ritox 35®, and Corn oil.

Another embodiment comprises cyclosporin A, Surfonic DA-4®, and Corn oil.

Another embodiment comprises cyclosporin A, Sunonic DA-6®, and Corn oil.

Another embodiment comprises cyclosporin A, Sunonic L46-7®, and Corn oil.

Another embodiment comprises cyclosporin A, Sunonic POA®, and Corn oil.

Another embodiment comprises cyclosporin A, Synthrapol KB®, and Corn oil.

Another embodiment comprises cyclosporin A, Teginacid®, and Corn oil.

Another embodiment comprises cyclosporin A, Teginacid C®, and Corn oil.

Another embodiment comprises cyclosporin A, Tegotens EC 11®, and Corn oil.

Another embodiment comprises cyclosporin A, Tinegal NA®, and Corn oil.

Another embodiment comprises cyclosporin A, Tomadol 400®, and Corn oil.

Another embodiment comprises cyclosporin A, Tomadol 600®, and Corn oil.

Another embodiment comprises cyclosporin A, Tomadol 900®, and Corn oil.

Another embodiment comprises cyclosporin A, Uniperol O®, and Corn oil.

Another embodiment comprises cyclosporin A, Witconol SN Series®, and Corn oil.

Another embodiment comprises cyclosporin A, an Ethoxylated Alkylphenol, and Corn oil.

Another embodiment comprises cyclosporin A, Antarox LF-222®, and Corn oil.

Another embodiment comprises cyclosporin A, Atlox 775®, and Corn oil.

Another embodiment comprises cyclosporin A, Caloxylate N-9®, and Corn oil.

Another embodiment comprises cyclosporin A, Canasol NF-1000®, and Corn oil.

Another embodiment comprises cyclosporin A, Canasol NF-3000®, and Corn oil.

Another embodiment comprises cyclosporin A, Canasol NF-3070®, and Corn oil.

Another embodiment comprises cyclosporin A, Canasol OF 1670®, and Com oil.

Another embodiment comprises cyclosporin A, Canasol OF 2570®, and Corn oil.

Another embodiment comprises cyclosporin A, Canasol OF 4070®, and Corn oil.

Another embodiment comprises cyclosporin A, Chemax DNP-8®, and Corn oil.

Another embodiment comprises cyclosporin A, Chemax DNP-18®, and Corn oil.

Another embodiment comprises cyclosporin A, Chemax DNP-150/50®, and Corn oil.

Another embodiment comprises cyclosporin A, DeSonic 1.5N®, and Com oil.

Another embodiment comprises cyclosporin A, DeSonic 4N®, and Corn oil.

Another embodiment comprises cyclosporin A, DeSonic 5N®, and Corn oil.

Another embodiment comprises cyclosporin A, DeSonic 6D®, and Corn oil.

Another embodiment comprises cyclosporin A, DeSonic 6N®, and Corn oil.

Another embodiment comprises cyclosporin A, DeSonic 7N®, and Corn oil.

Another embodiment comprises cyclosporin A, DeSonic 9N®, and Corn oil.

Another embodiment comprises cyclosporin A, DeSonic 10D®, and Corn oil.

Another embodiment comprises cyclosporin A, DeSonic 11N®, and Corn oil.

Another embodiment comprises cyclosporin A, DeSonic 12N®, and Corn oil.

Another embodiment comprises cyclosporin A, DeSonic 15N®, and Corn oil.

Another embodiment comprises cyclosporin A, DeSonic 20N®, and Corn oil.

Another embodiment comprises cyclosporin A, Eccoscour RC®, and Corn oil.

Another embodiment comprises cyclosporin A, Eccoterge EO-100®, and Corn oil.

Another embodiment comprises cyclosporin A, Emulsifier 632/90%®, and Corn oil.

Another embodiment comprises cyclosporin A, Geronol AG-821®, and Corn oil.

Another embodiment comprises cyclosporin A, Gradonic N-95®, and Corn oil.

Another embodiment comprises cyclosporin A, Hetoxide NP-4®, and Corn oil.

Another embodiment comprises cyclosporin A, Hetoxide NP-30®, and Corn oil.

Another embodiment comprises cyclosporin A, Hostapal N-100®, and Corn oil.

Another embodiment comprises cyclosporin A, Hostapal N-110®, and Corn oil.

Another embodiment comprises cyclosporin A, Igepal CTA-639W®, and Corn oil.

Another embodiment comprises cyclosporin A, Igepal DAP-9®, and Corn oil.

Another embodiment comprises cyclosporin A, Igepal OD-410®, and Corn oil.

Another embodiment comprises cyclosporin A, Igepal SS-837®, and Corn oil.

Another embodiment comprises cyclosporin A, Lipocol NP-9 USP®, and Corn oil.

Another embodiment comprises cyclosporin A, Macol DNP-10®, and Corn oil.

Another embodiment comprises cyclosporin A, Marlophen NP 5®, and Corn oil.

Another embodiment comprises cyclosporin A, Marlophen P 1®, and Corn oil.

Another embodiment comprises cyclosporin A, Surfonic NB®, and Corn oil.

Another embodiment comprises cyclosporin A, Surfonic OPB-307®, and Corn oil.

Another embodiment comprises cyclosporin A, Surfonic OPB-407®, and Corn oil.

Another embodiment comprises cyclosporin A, Syn Fac 334®, and Corn oil.

Another embodiment comprises cyclosporin A, Syn Fac 8216®, and Corn oil.

Another embodiment comprises cyclosporin A, Triton N-57®, and Corn oil.

Another embodiment comprises cyclosporin A, Trycol 6956®, and Corn oil.

Another embodiment comprises cyclosporin A, Trycol 6961®, and Corn oil.

Another embodiment comprises cyclosporin A, Trycol 6964®, and Corn oil.

Another embodiment comprises cyclosporin A, Trycol 6969®, and Corn oil.

Another embodiment comprises cyclosporin A, Trycol 6974®, and Corn oil.

Another embodiment comprises cyclosporin A, Witbreak DRB-127®, and Corn oil.

Another embodiment comprises cyclosporin A, Witbreak DRB-127®, and Corn oil.

Another embodiment comprises cyclosporin A, Witconol NP Series®, and Corn oil.

Another embodiment comprises cyclosporin A, an Ethoxylated Aryl Phenol, and Corn oil.

Another embodiment comprises cyclosporin A, Soprophor BSU®, and Corn oil.

Another embodiment comprises cyclosporin A, Soprophor CY/8®, and Corn oil.

Another embodiment comprises cyclosporin A, Soprophor S/25®, and Corn oil.

Another embodiment comprises cyclosporin A, Witconol NIO®, and Corn oil.

Another embodiment comprises cyclosporin A, Witconol NIW®, and Corn oil.

Another embodiment comprises cyclosporin A, Witconol S-100®, and Corn oil.

Another embodiment comprises cyclosporin A, an Ethoxylated Fatty Acid, and Corn oil.

Another embodiment comprises cyclosporin A, Aldo PGHMS®, and Corn oil.

Another embodiment comprises cyclosporin A, Alkamuls TO-15/HR®, and Corn oil.

Another embodiment comprises cyclosporin A, Armotan AL-69-66®, and Corn oil.

Another embodiment comprises cyclosporin A, Cerasynt 840®, and Corn oil.

Another embodiment comprises cyclosporin A, Cerasynt 945®, and Corn oil.

Another embodiment comprises cyclosporin A, Crystal Inhibitor No. 5®, and Corn oil.

Another embodiment comprises cyclosporin A, DeThox Acid L-9®, and Corn oil.

Another embodiment comprises cyclosporin A, DeThox Acid S-8®, and Corn oil.

Another embodiment comprises cyclosporin A, Ethofat 242/25®, and Corn oil.

Another embodiment comprises cyclosporin A, Hydropalat 65®, and Corn oil.

Another embodiment comprises cyclosporin A, Lipo EGMS®, and Corn oil.

Another embodiment comprises cyclosporin A, Lipopeg 2 DL®, and Corn oil.

Another embodiment comprises cyclosporin A, Lipopeg 4 DL®, and Corn oil.

Another embodiment comprises cyclosporin A, Lipopeg 4-L®, and Corn oil.

Another embodiment comprises cyclosporin A, Lipopeg 39-S®, and Corn oil.

Another embodiment comprises cyclosporin A, Lipopeg 4-S®, and Corn oil.

Another embodiment comprises cyclosporin A, Lipopeg 10-S®, and Corn oil.

Another embodiment comprises cyclosporin A, Lipopeg 100-S®, and Corn oil.

Another embodiment comprises cyclosporin A, Lipopeg 6000 DS®, and Corn oil.

Another embodiment comprises cyclosporin A, Lumulse 40-L®, and Corn oil.

Another embodiment comprises cyclosporin A, Lumulse 40-S®, and Corn oil.

Another embodiment comprises cyclosporin A, Lumulse 42-L®, and Corn oil.

Another embodiment comprises cyclosporin A, Lumulse 42-S®, and Corn oil.

Another embodiment comprises cyclosporin A, Lumulse 100-S®, and Corn oil.

Another embodiment comprises cyclosporin A, Lumulse 602-S®, and Corn oil.

Another embodiment comprises cyclosporin A, Magrabar PGE-20-0®, and Corn oil.

Another embodiment comprises cyclosporin A, Magrabar PGE-20L®, and Corn oil.

Another embodiment comprises cyclosporin A, Magrabar PGE-20T®, and Corn oil.

Another embodiment comprises cyclosporin A, Magrabar PGE-22-0®, and Corn oil.

Another embodiment comprises cyclosporin A, Magrabar PGE-22L®, and Corn oil.

Another embodiment comprises cyclosporin A, Magrabar PGE-22T®, and Corn oil.

Another embodiment comprises cyclosporin A, Magrabar PGE-40-0®, and Corn oil.

Another embodiment comprises cyclosporin A, Magrabar PGE-40L®, and Corn oil.

Another embodiment comprises cyclosporin A, Magrabar PGE-40T®, and Corn oil.

Another embodiment comprises cyclosporin A, Magrabar PGE-42-0®, and Corn oil.

Another embodiment comprises cyclosporin A, Magrabar PGE-42L®, and Corn oil.

Another embodiment comprises cyclosporin A, Magrabar PGE-42T®, and Corn oil.

Another embodiment comprises cyclosporin A, Magrabar PGE-60-0®, and Corn oil.

Another embodiment comprises cyclosporin A, Magrabar PGE-60L®, and Corn oil.

Another embodiment comprises cyclosporin A, Magrabar PGE-60T®, and Corn oil.

Another embodiment comprises cyclosporin A, Magrabar PGE-62-0®, and Corn oil.

Another embodiment comprises cyclosporin A, Magrabar PGE-62L®, and Corn oil.

Another embodiment comprises cyclosporin A, Magrabar PGE-62T®, and Corn oil.

Another embodiment comprises cyclosporin A, Mapeg S-40K®, and Corn oil.

Another embodiment comprises cyclosporin A, Marlowet OTS®, and Corn oil.

Another embodiment comprises cyclosporin A, Naturechem PGR®, and Corn oil.

Another embodiment comprises cyclosporin A, PG No. 4®, and Corn oil.

Another embodiment comprises cyclosporin A, Renex 20®, and Corn oil.

Another embodiment comprises cyclosporin A, Ritox 52®, and Corn oil.

Another embodiment comprises cyclosporin A, Ritox 53®, and Corn oil.

Another embodiment comprises cyclosporin A, Ritox 59®, and Corn oil.

Another embodiment comprises cyclosporin A, Surfax 8916/A®, and Corn oil.

Another embodiment comprises cyclosporin A, Tego Acid S 40 P®, and Corn oil.

Another embodiment comprises cyclosporin A, Tego Acid S 100 P®, and Corn oil.

Another embodiment comprises cyclosporin A, Tween 20®, and Corn oil.

Another embodiment comprises cyclosporin A, Volpo 131®, and Corn oil.

Another embodiment comprises cyclosporin A, an Ethoxylated Fatty Ester or Oil, and Corn oil.

Another embodiment comprises cyclosporin A, Acconon 6-C10®, and Corn oil.

Another embodiment comprises cyclosporin A, Acconon CC-6®, and Corn oil.

Another embodiment comprises cyclosporin A, Acconon CO-7®, and Corn oil.

Another embodiment comprises cyclosporin A, Aldosperse 40/60 FG®, and Corn oil.

Another embodiment comprises cyclosporin A, Aldosperse ML-23®, and Corn oil.

Another embodiment comprises cyclosporin A, Aldosperse MS-20 FG®, and Corn oil.

Another embodiment comprises cyclosporin A, Alkamuls EL-620®, and Corn oil.

Another embodiment comprises cyclosporin A, Alkamuls EL-719®, and Corn oil.

Another embodiment comprises cyclosporin A, Alkamuls EL-985®, and Corn oil.

Another embodiment comprises cyclosporin A, Arlatone G®, and Corn oil.

Another embodiment comprises cyclosporin A, Arlatone T®, and Corn oil.

Another embodiment comprises cyclosporin A, Atlas G-1045A®, and Corn oil.

Another embodiment comprises cyclosporin A, Atlas G-1086®, and Corn oil.

Another embodiment comprises cyclosporin A, Atlas G-1087®, and Corn oil.

Another embodiment comprises cyclosporin A, Atlas G-1089®, and Corn oil.

Another embodiment comprises cyclosporin A, Atlas G-1096®, and Corn oil.

Another embodiment comprises cyclosporin A, Atlas G-1292®, and Corn oil.

Another embodiment comprises cyclosporin A, Atlas G-1293®, and Corn oil.

Another embodiment comprises cyclosporin A, Atlas G-1300®, and Corn oil.

Another embodiment comprises cyclosporin A, Atlas G-7076®, and Corn oil.

Another embodiment comprises cyclosporin A, Capmul EMG®, and Corn oil.

Another embodiment comprises cyclosporin A, Chemonic CO-40®, and Corn oil.

Another embodiment comprises cyclosporin A, Chemonic LI-3®, and Corn oil.

Another embodiment comprises cyclosporin A, Chemonic LI-7®, and Corn oil.

Another embodiment comprises cyclosporin A, Cirrasol GM®, and Corn oil.

Another embodiment comprises cyclosporin A, Cremophor CO 40®, and Corn oil.

Another embodiment comprises cyclosporin A, Cremophor CO 410®, and Corn oil.

Another embodiment comprises cyclosporin A, Cremophor EL®, and Corn oil.

Another embodiment comprises cyclosporin A, Cremophor GC7®, and Corn oil.

Another embodiment comprises cyclosporin A, Cremophor RH-40®, and Corn oil.

Another embodiment comprises cyclosporin A, Crovol A-40®, and Corn oil.

Another embodiment comprises cyclosporin A, Crovol A-70®, and Corn oil.

Another embodiment comprises cyclosporin A, Crovol M-70®, and Corn oil.

Another embodiment comprises cyclosporin A, Crovol PK-70®, and Corn oil.

Another embodiment comprises cyclosporin A, Cutina E-24®, and Corn oil.

Another embodiment comprises cyclosporin A, Dacospin 12-R®, and Corn oil.

Another embodiment comprises cyclosporin A, Dehymuls HRE-7®, and Corn oil.

Another embodiment comprises cyclosporin A, DeSonic 30C®, and Corn oil.

Another embodiment comprises cyclosporin A, DeSonic 36C®, and Corn oil.

Another embodiment comprises cyclosporin A, DeSonic 40C®, and Corn oil.

Another embodiment comprises cyclosporin A, Durfax 60®, and Corn oil.

Another embodiment comprises cyclosporin A, Durfax 65®, and Corn oil.

Another embodiment comprises cyclosporin A, Durfax 80®, and Corn oil.

Another embodiment comprises cyclosporin A, Durfax EOM®, and Corn oil.

Another embodiment comprises cyclosporin A, Eccoterge NF-2®, and Corn oil.

Another embodiment comprises cyclosporin A, Emulpon CO-360®, and Corn oil.

Another embodiment comprises cyclosporin A, Emulpon CO-550®, and Corn oil.

Another embodiment comprises cyclosporin A, Emulsogen EL®, and Corn oil.

Another embodiment comprises cyclosporin A, Emulsogen HCO 040®, and Corn oil.

Another embodiment comprises cyclosporin A, Emulsogen HCO 060®, and Corn oil.

Another embodiment comprises cyclosporin A, Emulsynt 1055®, and Corn oil.

Another embodiment comprises cyclosporin A, Ethox 3095®, and Corn oil.

Another embodiment comprises cyclosporin A, Eumulgin RO-40®, and Corn oil.

Another embodiment comprises cyclosporin A, Genapol G-260®, and Corn oil.

Another embodiment comprises cyclosporin A, Glycosperse L-20®, and Corn oil.

Another embodiment comprises cyclosporin A, Glycosperse O-5®, and Corn oil.

Another embodiment comprises cyclosporin A, Glycosperse O-20®, and Corn oil.

Another embodiment comprises cyclosporin A, Glycosperse O-20 FG®, and Corn oil.

Another embodiment comprises cyclosporin A, Glycosperse S-20®, and Corn oil.

Another embodiment comprises cyclosporin A, Glycosperse S-20 FG®, and Corn oil.

Another embodiment comprises cyclosporin A, Glycosperse TS-20®, and Corn oil.

Another embodiment comprises cyclosporin A, Glycosperse TS-20 FG®, and Corn oil.

Another embodiment comprises cyclosporin A, Hetan SL®, and Corn oil.

Another embodiment comprises cyclosporin A, Hetan SC®, and Corn oil.

Another embodiment comprises cyclosporin A, Hetan SS®, and Corn oil.

Another embodiment comprises cyclosporin A, Hetoxide C-2®, and Corn oil.

Another embodiment comprises cyclosporin A, Hetoxide C-9®, and Corn oil.

Another embodiment comprises cyclosporin A, Hetoxide C-15®, and Corn oil.

Another embodiment comprises cyclosporin A, Hetoxide C-25®, and Corn oil.

Another embodiment comprises cyclosporin A, Hetoxide C-40®, and Corn oil.

Another embodiment comprises cyclosporin A, Hetoxide C-200®, and Corn oil.

Another embodiment comprises cyclosporin A, Hetoxide C-200-50%®, and Corn oil.

Another embodiment comprises cyclosporin A, Hetoxide GC-30®, and Corn oil.

Another embodiment comprises cyclosporin A, Hetoxide HC-60®, and Corn oil.

Another embodiment comprises cyclosporin A, Ice No. 2®, and Corn oil.

Another embodiment comprises cyclosporin A, Incrocas 30/40®, and Corn oil.

Another embodiment comprises cyclosporin A, Lexol EC®, and Corn oil.

Another embodiment comprises cyclosporin A, Lexol EO®, and Corn oil.

Another embodiment comprises cyclosporin A, Lipocol HCO-40®, and Corn oil.

Another embodiment comprises cyclosporin A, Lipocol HCO-60®, and Corn oil.

Another embodiment comprises cyclosporin A, Lipocol O-3 Special®, and Corn oil.

Another embodiment comprises cyclosporin A, Lipopeg 2-L®, and Corn oil.

Another embodiment comprises cyclosporin A, Lipopeg 4-DO®, and Corn oil.

Another embodiment comprises cyclosporin A, Lipopeg 4-DS®, and Corn oil.

Another embodiment comprises cyclosporin A, Lipovol GTB®, and Corn oil.

Another embodiment comprises cyclosporin A, Lonzest SML-20®, and Corn oil.

Another embodiment comprises cyclosporin A, Lonzest SMO-20®, and Corn oil.

Another embodiment comprises cyclosporin A, Lonzest SMS-20®, and Corn oil.

Another embodiment comprises cyclosporin A, Lonzest STO-20®, and Corn oil.

Another embodiment comprises cyclosporin A, Lonzest STS-20®, and Corn oil.

Another embodiment comprises cyclosporin A, Lumulse GR-40®, and Corn oil.

Another embodiment comprises cyclosporin A, Lumulse GRH-40®, and Corn oil.

Another embodiment comprises cyclosporin A, Lumulse POE (7) GML®, and Corn oil.

Another embodiment comprises cyclosporin A, Lumulse POE (12) Glyc®, and Corn oil.

Another embodiment comprises cyclosporin A, Lumulse POE (40) MS KP®, and Corn oil.

Another embodiment comprises cyclosporin A, Marlowet 4750®, and Corn oil.

Another embodiment comprises cyclosporin A, Marlowet LVS®, and Corn oil.

Another embodiment comprises cyclosporin A, Marlowet R 11®, and Corn oil.

Another embodiment comprises cyclosporin A, Marlowet R 40®, and Corn oil.

Another embodiment comprises cyclosporin A, Mazol 80 MGK®, and Corn oil.

Another embodiment comprises cyclosporin A, Nonionic Emulsifier T-9®, and Corn oil.

Another embodiment comprises cyclosporin A, Oronal LCG®, and Corn oil.

Another embodiment comprises cyclosporin A, Polyderm PPI-CO-200®, and Corn oil.

Another embodiment comprises cyclosporin A, Polyderm PPI-CO-40®, and Corn oil.

Another embodiment comprises cyclosporin A, Rewoderm LI 520-70®, and Corn oil.

Another embodiment comprises cyclosporin A, Ritapeg 150 DS®, and Corn oil.

Another embodiment comprises cyclosporin A, Softigen 767®, and Corn oil.

Another embodiment comprises cyclosporin A, Surfactol 318®, and Corn oil.

Another embodiment comprises cyclosporin A, Surfactol 365®, and Corn oil.

Another embodiment comprises cyclosporin A, Syn Lube 107®, and Corn oil.

Another embodiment comprises cyclosporin A, Syn Lube 728®, and Corn oil.

Another embodiment comprises cyclosporin A, Syn Lube 1632H®, and Corn oil.

Another embodiment comprises cyclosporin A, Syn Lube 6277-A®, and Corn oil.

Another embodiment comprises cyclosporin A, T-Det C-20®, and Corn oil.

Another embodiment comprises cyclosporin A, T-Det C-40®, and Corn oil.

Another embodiment comprises cyclosporin A, Tally 100 Plus®, and Corn oil.

Another embodiment comprises cyclosporin A, Uniperol EL®, and Corn oil.

Another embodiment comprises cyclosporin A, a Fatty Ester, and Corn oil.

Another embodiment comprises cyclosporin A, Actralube-Syn 147®, and Corn oil.

Another embodiment comprises cyclosporin A, Atlas G-1556®, and Corn oil.

Another embodiment comprises cyclosporin A, Atlas G-1564®, and Corn oil.

Another embodiment comprises cyclosporin A, Atlasol Base Oil S®, and Corn oil.

Another embodiment comprises cyclosporin A, Base ML®, and Corn oil.

Another embodiment comprises cyclosporin A, Base MT®, and Corn oil.

Another embodiment comprises cyclosporin A, Cerasynt 303®, and Corn oil.

Another embodiment comprises cyclosporin A, Dermol 1012®, and Corn oil.

Another embodiment comprises cyclosporin A, Kemester 4000®, and Corn oil.

Another embodiment comprises cyclosporin A, Lactipol S®, and Corn oil.

Another embodiment comprises cyclosporin A, Magrabar PGO®, and Corn oil.

Another embodiment comprises cyclosporin A, Mayco Base BFO®, and Corn oil.

Another embodiment comprises cyclosporin A, Methyl Linoleate®, and Corn oil.

Another embodiment comprises cyclosporin A, Pationic 122A®, and Corn oil.

Another embodiment comprises cyclosporin A, Pationic 138C®, and Corn oil.

Another embodiment comprises cyclosporin A, Pationic CSL®, and Corn oil.

Another embodiment comprises cyclosporin A, Pationic ISL®, and Corn oil.

Another embodiment comprises cyclosporin A, Pationic SBL®, and Corn oil.

Another embodiment comprises cyclosporin A, Pationic SSL®, and Corn oil.

Another embodiment comprises cyclosporin A, Ritasol®, and Corn oil.

Another embodiment comprises cyclosporin A, Tego Alkanol CS 20®, and Corn oil.

Another embodiment comprises cyclosporin A, Tego Alkanol L23 P®, and Corn oil.

Another embodiment comprises cyclosporin A, Tego Alkanol S2®, and Corn oil.

Another embodiment comprises cyclosporin A, Tego Alkanol S20 P®, and Corn oil.

Another embodiment comprises cyclosporin A, Triemulsifier 600 MS®, and Corn oil.

Another embodiment comprises cyclosporin A, a Glycerol Esters, and Corn oil.

Another embodiment comprises cyclosporin A, Agro #9 Wint SBO®, and Corn oil.

Another embodiment comprises cyclosporin A, Ahcovel Base 700®, and Com oil.

Another embodiment comprises cyclosporin A, Aldo HMS FG®, and Corn oil.

Another embodiment comprises cyclosporin A, Aldo MLD®, and Corn oil.

Another embodiment comprises cyclosporin A, Aldo MLD FG®, and Corn oil.

Another embodiment comprises cyclosporin A, Aldo MO FG®, and Corn oil.

Another embodiment comprises cyclosporin A, Aldo MS®, and Corn oil.

Another embodiment comprises cyclosporin A, Aldo MS FG®, and Corn oil.

Another embodiment comprises cyclosporin A, Aldo MS LG FG®, and Corn oil.

Another embodiment comprises cyclosporin A, Aldo MSO®, and Corn oil.

Another embodiment comprises cyclosporin A, Aldo MSD FG®, and Corn oil.

Another embodiment comprises cyclosporin A, Aldosperse O-20 FG®, and Corn oil.

Another embodiment comprises cyclosporin A, Aldosperse TS-20 FG®, and Corn oil.

Another embodiment comprises cyclosporin A, Aldosperse TS-40 FG®, and Corn oil.

Another embodiment comprises cyclosporin A, Arlacel 165®, and Corn oil.

Another embodiment comprises cyclosporin A, Arlacel 186®, and Corn oil.

Another embodiment comprises cyclosporin A, Capmul GMO®, and Corn oil.

Another embodiment comprises cyclosporin A, Capmul GMS®, and Corn oil.

Another embodiment comprises cyclosporin A, Caprol 3GC®, and Corn oil.

Another embodiment comprises cyclosporin A, Caprol 3GVS®, and Corn oil.

Another embodiment comprises cyclosporin A, Caprol 6G2S®, and Corn oil.

Another embodiment comprises cyclosporin A, Caprol 10G40®, and Corn oil.

Another embodiment comprises cyclosporin A, Caprol 10G100®, and Corn oil.

Another embodiment comprises cyclosporin A, Caprol ET®, and Corn oil.

Another embodiment comprises cyclosporin A, Caprol PGE860®, and Corn oil.

Another embodiment comprises cyclosporin A, Cerasynt 945®, and Corn oil.

Another embodiment comprises cyclosporin A, Cerasynt GMS®, and Corn oil.

Another embodiment comprises cyclosporin A, Cerasynt Q®, and Corn oil.

Another embodiment comprises cyclosporin A, Cerasynt SD®, and Corn oil.

Another embodiment comprises cyclosporin A, Cerasynt WM®, and Corn oil.

Another embodiment comprises cyclosporin A, Chemsperse 14®, and Corn oil.

Another embodiment comprises cyclosporin A, Cremophor GO-32®, and Corn oil.

Another embodiment comprises cyclosporin A, Cremophor GS11®, and Corn oil.

Another embodiment comprises cyclosporin A, Cremophor GS-32®, and Corn oil.

Another embodiment comprises cyclosporin A, Cutina KD-16®, and Corn oil.

Another embodiment comprises cyclosporin A, Dehymuls PGPH®, and Corn oil.

Another embodiment comprises cyclosporin A, Dermol DGDIS®, and Corn oil.

Another embodiment comprises cyclosporin A, Dermoi DGMIS®, and Corn oil.

Another embodiment comprises cyclosporin A, Dermol G-76®, and Corn oil.

Another embodiment comprises cyclosporin A, Dermol G-7DI®, and Corn oil.

Another embodiment comprises cyclosporin A, Dermol NGDI®, and Corn oil.

Another embodiment comprises cyclosporin A, Dermolan GLH®, and Corn oil.

Another embodiment comprises cyclosporin A, Drewmulse GMO®, and Corn oil.

Another embodiment comprises cyclosporin A, Drewpol 3-5-M®, and Corn oil.

Another embodiment comprises cyclosporin A, Durlac 100 W®, and Corn oil.

Another embodiment comprises cyclosporin A, Dur-Lo®, and Corn oil.

Another embodiment comprises cyclosporin A, Dynasan 118®, and Corn oil.

Another embodiment comprises cyclosporin A, EC-25®, and Corn oil.

Another embodiment comprises cyclosporin A, EM 40®, and Corn oil.

Another embodiment comprises cyclosporin A, Emerest 2400®, and Corn oil.

Another embodiment comprises cyclosporin A, Emerest 2452®, and Corn oil.

Another embodiment comprises cyclosporin A, Empilan G-26®, and Corn oil.

Another embodiment comprises cyclosporin A, Genapol TSM®, and Corn oil.

Another embodiment comprises cyclosporin A, Hostacerin DGI®, and Corn oil.

Another embodiment comprises cyclosporin A, Hostacerin DGL®, and Corn oil.

Another embodiment comprises cyclosporin A, Hostacerin DGMS®, and Corn oil.

Another embodiment comprises cyclosporin A, Hostacerin DGSB®, and Corn oil.

Another embodiment comprises cyclosporin A, Ice No. 2®, and Corn oil.

Another embodiment comprises cyclosporin A, Imwitor 742®, and Corn oil.

Another embodiment comprises cyclosporin A, Imwitor 780 K®, and Corn oil.

Another embodiment comprises cyclosporin A, Imwitor 960 Flakes®, and Corn oil.

Another embodiment comprises cyclosporin A, Isolan GI 34®, and Corn oil.

Another embodiment comprises cyclosporin A, Isolan GO 33®, and Corn oil.

Another embodiment comprises cyclosporin A, Kemester 1000®, and Corn oil.

Another embodiment comprises cyclosporin A, Kemester 2000®, and Corn oil.

Another embodiment comprises cyclosporin A, Kemester 2000®, and Corn oil.

Another embodiment comprises cyclosporin A, Kemester 6000SEO, and Corn oil.

Another embodiment comprises cyclosporin A, Lamecreme DGE 18®, and Corn oil.

Another embodiment comprises cyclosporin A, Lexemul 515®, and Corn oil.

Another embodiment comprises cyclosporin A, Lexemul 561®, and Corn oil.

Another embodiment comprises cyclosporin A, Lexemul AR®, and Corn oil.

Another embodiment comprises cyclosporin A, Lexemul AS®, and Corn oil.

Another embodiment comprises cyclosporin A, Lexemul GDL®, and Corn oil.

Another embodiment comprises cyclosporin A, Lexemul T®, and Corn oil.

Another embodiment comprises cyclosporin A, Lipomulse 165®, and Corn oil.

Another embodiment comprises cyclosporin A, Lumulse GML K®, and Corn oil.

Another embodiment comprises cyclosporin A, Lumulse GMO K®, and Corn oil.

Another embodiment comprises cyclosporin A, Lumulse GMR K®, and Corn oil.

Another embodiment comprises cyclosporin A, Lumulse GMT K®, and Corn oil.

Another embodiment comprises cyclosporin A, Magrabar GMC®, and Corn oil.

Another embodiment comprises cyclosporin A, Magrabar GMO-CK®, and Corn oil.

Another embodiment comprises cyclosporin A, Magrabar MDG-505®, and Corn oil.

Another embodiment comprises cyclosporin A, Magrabar PGO-315®, and Corn oil.

Another embodiment comprises cyclosporin A, Magrabar PGO-1010®, and Corn oil.

Another embodiment comprises cyclosporin A, Mazol 300K®, and Corn oil.

Another embodiment comprises cyclosporin A, Mazol GMO-K®, and Corn oil.

Another embodiment comprises cyclosporin A, Mazol GMS-K®, and Corn oil.

Another embodiment comprises cyclosporin A, Mazol PG031-K®, and Corn oil.

Another embodiment comprises cyclosporin A, Miglyol 812®, and Corn oil.

Another embodiment comprises cyclosporin A, Norfox 165C®, and Corn oil.

Another embodiment comprises cyclosporin A, Schercemol GMIS®, and Corn oil.

Another embodiment comprises cyclosporin A, Tegin®, and Corn oil.

Another embodiment comprises cyclosporin A, Tegin 4100 Pellets®, and Corn oil.

Another embodiment comprises cyclosporin A, Tegin M Pellets®, and Corn oil.

Another embodiment comprises cyclosporin A, Tegin OV®, and Corn oil.

Another embodiment comprises cyclosporin A, Teginacid H®, and Corn oil.

Another embodiment comprises cyclosporin A, Tego Cosmo P813®, and Corn oil.

Another embodiment comprises cyclosporin A, Wickenol 535®, and Corn oil.

Another embodiment comprises cyclosporin A, Witconol 14®, and Corn oil.

Another embodiment comprises cyclosporin A, Witconol 14®, and Corn oil.

Another embodiment comprises cyclosporin A, Witconol 14F®, and Corn oil.

Another embodiment comprises cyclosporin A, Witconol 18L®, and Corn oil.

Another embodiment comprises cyclosporin A, Witconol GOT®, and Corn oil.

Another embodiment comprises cyclosporin A, Witconol MST®, and Corn oil.

Another embodiment comprises cyclosporin A, Witconol RHT®, and Corn oil.

Another embodiment comprises cyclosporin A, a Glycol Ester, and Corn oil.

Another embodiment comprises cyclosporin A, Alkamuls 600 DO®, and Corn oil.

Another embodiment comprises cyclosporin A, Alkamuls SEG®, and Corn oil.

Another embodiment comprises cyclosporin A, Atlas EM-2®, and Corn oil.

Another embodiment comprises cyclosporin A, Cerasynt IP®, and Corn oil.

Another embodiment comprises cyclosporin A, Cerasynt M®, and Corn oil.

Another embodiment comprises cyclosporin A, Cerasynt MN®, and Corn oil.

Another embodiment comprises cyclosporin A, Cerasynt PA®, and Corn oil.

Another embodiment comprises cyclosporin A, Chemsperse EGDS®, and Corn oil.

Another embodiment comprises cyclosporin A, Chemsperse EGMS®, and Corn oil.

Another embodiment comprises cyclosporin A, Colonial Monolaurin®, and Corn oil.

Another embodiment comprises cyclosporin A, DeMuls SGE-95®, and Corn oil.

Another embodiment comprises cyclosporin A, Eccoterge 200®, and Corn oil.

Another embodiment comprises cyclosporin A, Emerest 2380®, and Corn oil.

Another embodiment comprises cyclosporin A, Ethox 2610®, and Corn oil.

Another embodiment comprises cyclosporin A, Ethox DO-9®, and Corn oil.

Another embodiment comprises cyclosporin A, Ethox DO-14®, and Corn oil.

Another embodiment comprises cyclosporin A, Ethox SO-9®, and Corn oil.

Another embodiment comprises cyclosporin A, Fizul MD-318®, and Corn oil.

Another embodiment comprises cyclosporin A, Genapol EGDS-VHP®, and Corn oil.

Another embodiment comprises cyclosporin A, Genapol TS Powder®, and Corn oil.

Another embodiment comprises cyclosporin A, Hostacerin WO®, and Corn oil.

Another embodiment comprises cyclosporin A, Inversol 140®, and Corn oil.

Another embodiment comprises cyclosporin A, Kemester 104®, and Corn oil.

Another embodiment comprises cyclosporin A, Kemester 205®, and Corn oil.

Another embodiment comprises cyclosporin A, Kemester 226®, and Corn oil.

Another embodiment comprises cyclosporin A, Kemester 5221SE®, and Corn oil.

Another embodiment comprises cyclosporin A, Kemester EGDS®, and Corn oil.

Another embodiment comprises cyclosporin A, Lexemul EGDS®, and Corn oil.

Another embodiment comprises cyclosporin A, Lexemul EGMS®, and Corn oil.

Another embodiment comprises cyclosporin A, Lexemul P®, and Corn oil.

Another embodiment comprises cyclosporin A, Lipo DGLS®, and Corn oil.

Another embodiment comprises cyclosporin A, Lipo EGDS®, and Corn oil.

Another embodiment comprises cyclosporin A, Lipo PGMS®, and Corn oil.

Another embodiment comprises cyclosporin A, Liposorb S-4®, and Corn oil.

Another embodiment comprises cyclosporin A, Liposorb TO-20®, and Corn oil.

Another embodiment comprises cyclosporin A, Lumulse PGC®, and Corn oil.

Another embodiment comprises cyclosporin A, Mackester EGDS®, and Corn oil.

Another embodiment comprises cyclosporin A, Mackester EGMS®, and Corn oil.

Another embodiment comprises cyclosporin A, Mackester GSTP®, and Corn oil.

Another embodiment comprises cyclosporin A, Mackester Series®, and Corn oil.

Another embodiment comprises cyclosporin A, Magrabar PDG-5®, and Corn oil.

Another embodiment comprises cyclosporin A, Mapeg 6000 DS®, and Corn oil.

Another embodiment comprises cyclosporin A, Marlowet 4702®, and Corn oil.

Another embodiment comprises cyclosporin A, Monalube 305®, and Corn oil.

Another embodiment comprises cyclosporin A, Monalube 310®, and Corn oil.

Another embodiment comprises cyclosporin A, Monalube 315®, and Corn oil.

Another embodiment comprises cyclosporin A, Monalube 320®, and Corn oil.

Another embodiment comprises cyclosporin A, Monalube 325®, and Corn oil.

Another embodiment comprises cyclosporin A, Monalube 330®, and Corn oil.

Another embodiment comprises cyclosporin A, Naturechem PGHS®, and Corn oil.

Another embodiment comprises cyclosporin A, Polycastorol PLO-840®, and Corn oil.

Another embodiment comprises cyclosporin A, Polytex 10M®, and Corn oil.

Another embodiment comprises cyclosporin A, Ritasynt IP®, and Corn oil.

Another embodiment comprises cyclosporin A, Ross Chem PEG 600 DT®, and Corn oil.

Another embodiment comprises cyclosporin A, Schercemol PGMS®, and Corn oil.

Another embodiment comprises cyclosporin A, Sponto H-44C®, and Corn oil.

Another embodiment comprises cyclosporin A, Tegin G®, and Corn oil.

Another embodiment comprises cyclosporin A, Witbreak DGE-182®, and Corn oil.

Another embodiment comprises cyclosporin A, Witbreak DGE-182®, and Corn oil.

Another embodiment comprises cyclosporin A, Witbreak DRA-21®, and Corn oil.

Another embodiment comprises cyclosporin A, Witbreak DRA-21®, and Corn oil.

Another embodiment comprises cyclosporin A, Witbreak DRA-50®, and Corn oil.

Another embodiment comprises cyclosporin A, Witbreak DRA-50®, and Corn oil.

Another embodiment comprises cyclosporin A, Witconol F26-46®, and Corn oil.

Another embodiment comprises cyclosporin A, Witconol H-32®, and Corn oil.

Another embodiment comprises cyclosporin A, Witconol H-33®, and Corn oil.

Another embodiment comprises cyclosporin A, Witconol H-35A®, and Corn oil.

Another embodiment comprises cyclosporin A, Witconol RHP®, and Corn oil.

Another embodiment comprises cyclosporin A, a Lanolin-based Derivative, and Corn oil.

Another embodiment comprises cyclosporin A, Amerchol CAB®, and Corn oil.

Another embodiment comprises cyclosporin A, Amerchol L-101®, and Corn oil.

Another embodiment comprises cyclosporin A, Amerlate LFA-LO®, and Corn oil.

Another embodiment comprises cyclosporin A, Amerlate P®, and Corn oil.

Another embodiment comprises cyclosporin A, Barre Common Degras®, and Corn oil.

Another embodiment comprises cyclosporin A, Cholesterol NF®, and Corn oil.

Another embodiment comprises cyclosporin A, Crodalan AWS®, and Corn oil.

Another embodiment comprises cyclosporin A, Crodalan LA®, and Corn oil.

Another embodiment comprises cyclosporin A, Emery 1650®, and Corn oil.

Another embodiment comprises cyclosporin A, Emery 1650®, and Corn oil.

Another embodiment comprises cyclosporin A, Emery 1740®, and Corn oil.

Another embodiment comprises cyclosporin A, Emery 1740®, and Corn oil.

Another embodiment comprises cyclosporin A, Forlan 500®, and Corn oil.

Another embodiment comprises cyclosporin A, Forlan L®, and Corn oil.

Another embodiment comprises cyclosporin A, Laneto 50®, and Corn oil.

Another embodiment comprises cyclosporin A, Laneto 100®, and Corn oil.

Another embodiment comprises cyclosporin A, Laneto AWS®, and Corn oil.

Another embodiment comprises cyclosporin A, Lanfrax 1776®, and Corn oil.

Another embodiment comprises cyclosporin A, Lanfrax 1776®, and Corn oil.

Another embodiment comprises cyclosporin A, Lanogel 21®, and Corn oil.

Another embodiment comprises cyclosporin A, Lipolan®, and Corn oil.

Another embodiment comprises cyclosporin A, Lipolan 31®, and Corn oil.

Another embodiment comprises cyclosporin A, OHlan®, and Corn oil.

Another embodiment comprises cyclosporin A, Polychol 5®, and Corn oil.

Another embodiment comprises cyclosporin A, Polychol 15®, and Corn oil.

Another embodiment comprises cyclosporin A, Ritacetyl®, and Corn oil.

Another embodiment comprises cyclosporin A, Ritachol®, and Corn oil.

Another embodiment comprises cyclosporin A, Ritahydrox®, and Corn oil.

Another embodiment comprises cyclosporin A, Ritalafa®, and Corn oil.

Another embodiment comprises cyclosporin A, Ritalan®, and Corn oil.

Another embodiment comprises cyclosporin A, Ritalan AWS®, and Corn oil.

Another embodiment comprises cyclosporin A, Ritalan C®, and Corn oil.

Another embodiment comprises cyclosporin A, Ritawax®, and Corn oil.

Another embodiment comprises cyclosporin A, Ritawax AEO®, and Corn oil.

Another embodiment comprises cyclosporin A, Ritawax ALA®, and Corn oil.

Another embodiment comprises cyclosporin A, Solan/Solan 50/Super Solan®, and Corn oil.

Another embodiment comprises cyclosporin A, Super Hartolan/Hartolan®, and Corn oil.

Another embodiment comprises cyclosporin A, Supersat AWS-4®, and Corn oil.

Another embodiment comprises cyclosporin A, Supersat AWS-24®, and Corn oil.

Another embodiment comprises cyclosporin A, a Lecithin or Lecithin Derivative, and Corn oil.

Another embodiment comprises cyclosporin A, Alcolec®, and Corn oil.

Another embodiment comprises cyclosporin A, Lecithin®, and Corn oil.

Another embodiment comprises cyclosporin A, Lexin K®, and Corn oil.

Another embodiment comprises cyclosporin A, Natipide®, and Corn oil.

Another embodiment comprises cyclosporin A, a Lignin or Lignin Derivative, and Corn oil.

Another embodiment comprises cyclosporin A, Diwatex XP 9®, and Corn oil.

Another embodiment comprises cyclosporin A, Dynasperse LCD®, and Corn oil.

Another embodiment comprises cyclosporin A, Indulin SAL®, and Corn oil.

Another embodiment comprises cyclosporin A, Indulin W-1®, and Corn oil.

Another embodiment comprises cyclosporin A, Indulin W-5®, and Corn oil.

Another embodiment comprises cyclosporin A, Lignosol FTA®, and Corn oil.

Another embodiment comprises cyclosporin A, Lignosol SFX-65®, and Corn oil.

Another embodiment comprises cyclosporin A, Marasperse 52 CP®, and Corn oil.

Another embodiment comprises cyclosporin A, Marasperse AG®, and Corn oil.

Another embodiment comprises cyclosporin A, Marasperse CBOS-4®, and Corn oil.

Another embodiment comprises cyclosporin A, Ufoxane 2®, and Corn oil.

Another embodiment comprises cyclosporin A, a Methyl Ester, and Corn oil.

Another embodiment comprises cyclosporin A, E.B. Cleaner AK®, and Corn oil.

Another embodiment comprises cyclosporin A, Oleocal ME-70®, and Corn oil.

Another embodiment comprises cyclosporin A, Trade Name Company®, and Corn oil.

Another embodiment comprises cyclosporin A, Methyl Esters (cont'd)®, and Corn oil.

Another embodiment comprises cyclosporin A, Oleocal ME-92®, and Corn oil.

Another embodiment comprises cyclosporin A, Oleocal ME-112®, and Corn oil.

Another embodiment comprises cyclosporin A, Oleocal ME-130®, and Corn oil.

Another embodiment comprises cyclosporin A, a Monoglyceride or a Derivative thereof, and Corn oil.

Another embodiment comprises cyclosporin A, Dynacet 211®, and Corn oil.

Another embodiment comprises cyclosporin A, Hetsorb S-20®, and Corn oil.

Another embodiment comprises cyclosporin A, Imwitor 191®, and Corn oil.

Another embodiment comprises cyclosporin A, Imwitor 370®, and Corn oil.

Another embodiment comprises cyclosporin A, Imwitor 375®, and Corn oil.

Another embodiment comprises cyclosporin A, Imwitor 900®, and Corn oil.

Another embodiment comprises cyclosporin A, Imwitor 945®, and Corn oil.

Another embodiment comprises cyclosporin A, Imwitor 2020®, and Corn oil.

Another embodiment comprises cyclosporin A, Kemester 5500®, and Corn oil.

Another embodiment comprises cyclosporin A, Kemester 6000®, and Corn oil.

Another embodiment comprises cyclosporin A, Magrabar GMC®, and Corn oil.

Another embodiment comprises cyclosporin A, Magrabar GMO-CK®, and Corn oil.

Another embodiment comprises cyclosporin A, Magrabar GPC-10®, and Corn oil.

Another embodiment comprises cyclosporin A, Magrabar MDG-5050®, and Corn oil.

Another embodiment comprises cyclosporin A, Monalube 335®, and Corn oil.

Another embodiment comprises cyclosporin A, Monoglycerides Glyceryl Monestearate Archer Daniels Midland Company®, and Corn oil.

Another embodiment comprises cyclosporin A, Rita GMS®, and Corn oil.

Another embodiment comprises cyclosporin A, Ritamulse SCG®, and Corn oil.

Another embodiment comprises cyclosporin A, Softigen 701®, and Corn oil.

Another embodiment comprises cyclosporin A, Tally 100 Plus®, and Corn oil.

Another embodiment comprises cyclosporin A, a Polyethylene Glycol, and Corn oil.

Another embodiment comprises cyclosporin A, Emulgade PL 68/50®, and Corn oil.

Another embodiment comprises cyclosporin A, Lumulse PEG®, and Corn oil.

Another embodiment comprises cyclosporin A, Rhodasurf PEG-400®, and Corn oil.

Another embodiment comprises cyclosporin A, Rhodasurf PEG-600®, and Corn oil.

Another embodiment comprises cyclosporin A, Witconol PEG-400®, and Corn oil.

Another embodiment comprises cyclosporin A, a Polymeric Surfactant, and Corn oil.

Another embodiment comprises cyclosporin A, Acritamer PNC-EG®, and Corn oil.

Another embodiment comprises cyclosporin A, Ag-Rho DEP-775®, and Corn oil.

Another embodiment comprises cyclosporin A, APG 325N Glycoside®, and Corn oil.

Another embodiment comprises cyclosporin A, Aristoflex AVC®, and Corn oil.

Another embodiment comprises cyclosporin A, Aristoflex HMB®, and Corn oil.

Another embodiment comprises cyclosporin A, Burco NPS-225®, and Corn oil.

Another embodiment comprises cyclosporin A, Burco NPS-816®, and Corn oil.

Another embodiment comprises cyclosporin A, Chemccinate 5603®, and Corn oil.

Another embodiment comprises cyclosporin A, Cosmedia Guar C-261 N®, and Corn oil.

Another embodiment comprises cyclosporin A, Gantrez S-95®, and Corn oil.

Another embodiment comprises cyclosporin A, Glucopon 220 UP®, and Corn oil.

Another embodiment comprises cyclosporin A, Glucopon 225 DK®, and Corn oil.

Another embodiment comprises cyclosporin A, Glucopon 425 N®, and Corn oil.

Another embodiment comprises cyclosporin A, Glucopon 600 UP®, and Corn oil.

Another embodiment comprises cyclosporin A, Glucopon 625 UP®, and Corn oil.

Another embodiment comprises cyclosporin A, Pemulen 1621®, and Corn oil.

Another embodiment comprises cyclosporin A, Pemulen 1622®, and Corn oil.

Another embodiment comprises cyclosporin A, Pemulen TR-1®, and Corn oil.

Another embodiment comprises cyclosporin A, Pemulen TR-2®, and Corn oil.

Another embodiment comprises cyclosporin A, Plantacare 818®, and Corn oil.

Another embodiment comprises cyclosporin A, Plantapon LGC Sorb®, and Corn oil.

Another embodiment comprises cyclosporin A, Plantaren 1200N®, and Corn oil.

Another embodiment comprises cyclosporin A, Plantaren 2000N®, and Corn oil.

Another embodiment comprises cyclosporin A, Viscolam AT 64®, and Corn oil.

Another embodiment comprises cyclosporin A, Viscolam AT 64P®, and Corn oil.

Another embodiment comprises cyclosporin A, Viscolam AT 100®, and Corn oil.

Another embodiment comprises cyclosporin A, Viscolam MAC 7®, and Corn oil.

Another embodiment comprises cyclosporin A, Viscolam SMC 20®, and Corn oil.

Another embodiment comprises cyclosporin A, Witbreak RTC-323®, and Corn oil.

Another embodiment comprises cyclosporin A, WSI 3700®, and Corn oil.

Another embodiment comprises cyclosporin A, a Propoxylated or Ethoxylated Fatty Acid, Alcohol, or Alkyl Phenol, and Corn oil.

Another embodiment comprises cyclosporin A, Antarox M-60®, and Corn oil.

Another embodiment comprises cyclosporin A, Antarox LF-224®, and Corn oil.

Another embodiment comprises cyclosporin A, Burcomul DFE-45®, and Corn oil.

Another embodiment comprises cyclosporin A, Burcoterge LFE-1000®, and Corn oil.

Another embodiment comprises cyclosporin A, Chemal LF-25B®, and Corn oil.

Another embodiment comprises cyclosporin A, Chemal LF-40B®, and Corn oil.

Another embodiment comprises cyclosporin A, Dehypon LS-36®, and Corn oil.

Another embodiment comprises cyclosporin A, Dehypon LS-36®, and Corn oil.

Another embodiment comprises cyclosporin A, Dehypon LS-54®, and Corn oil.

Another embodiment comprises cyclosporin A, Dehypon LS-54®, and Corn oil.

Another embodiment comprises cyclosporin A, Delonic 100 VLF®, and Corn oil.

Another embodiment comprises cyclosporin A, Delonic LF-60 MOD®, and Corn oil.

Another embodiment comprises cyclosporin A, Empiderm B®, and Corn oil.

Another embodiment comprises cyclosporin A, Ethylan 1206®, and Corn oil.

Another embodiment comprises cyclosporin A, Ethylan NS-500K®, and Corn oil.

Another embodiment comprises cyclosporin A, Ethylan NS-500LQ®, and Corn oil.

Another embodiment comprises cyclosporin A, Genapol 1392®, and Corn oil.

Another embodiment comprises cyclosporin A, Genapol 2317®, and Corn oil.

Another embodiment comprises cyclosporin A, Genapol 26EP710®, and Corn oil.

Another embodiment comprises cyclosporin A, Genapol EP 1022®, and Corn oil.

Another embodiment comprises cyclosporin A, Genapol EP 1024®, and Corn oil.

Another embodiment comprises cyclosporin A, Genapol EP 6068®, and Corn oil.

Another embodiment comprises cyclosporin A, Genapol NP915®, and Corn oil.

Another embodiment comprises cyclosporin A, Kieralon MFB®, and Corn oil.

Another embodiment comprises cyclosporin A, Lumisolve CSA-80 V®, and Corn oil.

Another embodiment comprises cyclosporin A, Marlowet 5001®, and Corn oil.

Another embodiment comprises cyclosporin A, Marlox FK 64®, and Corn oil.

Another embodiment comprises cyclosporin A, Marlox MO 124®, and Corn oil.

Another embodiment comprises cyclosporin A, Marlox S 58®, and Corn oil.

Another embodiment comprises cyclosporin A, Nonatell 1003®, and Corn oil.

Another embodiment comprises cyclosporin A, Nonatell 1038®, and Corn oil.

Another embodiment comprises cyclosporin A, Nonatell 1052®, and Corn oil.

Another embodiment comprises cyclosporin A, Nonatell 1061®, and Corn oil.

Another embodiment comprises cyclosporin A, Nonatell 1075®, and Corn oil.

Another embodiment comprises cyclosporin A, Nonatell 1088®, and Corn oil.

Another embodiment comprises cyclosporin A, Nonatell 1123®, and Corn oil.

Another embodiment comprises cyclosporin A, Nonatell 1153®, and Corn oil.

Another embodiment comprises cyclosporin A, Nonatell 1161®, and Corn oil.

Another embodiment comprises cyclosporin A, Nonatell 1172®, and Corn oil.

Another embodiment comprises cyclosporin A, Nonatell 1181®, and Corn oil.

Another embodiment comprises cyclosporin A, Nonfox 36®, and Corn oil.

Another embodiment comprises cyclosporin A, Procetyl AWS®, and Corn oil.

Another embodiment comprises cyclosporin A, Sandoxylate SX 412®, and Corn oil.

Another embodiment comprises cyclosporin A, Sandoxylate SX 418®, and Corn oil.

Another embodiment comprises cyclosporin A, Surfonic JL-80X®, and Corn oil.

Another embodiment comprises cyclosporin A, Surfonic JL-80X-B1®, and Corn oil.

Another embodiment comprises cyclosporin A, Sunonic L4-29X®, and Corn oil.

Another embodiment comprises cyclosporin A, Surfonic LF®, and Corn oil.

Another embodiment comprises cyclosporin A, T-Det A826®, and Corn oil.

Another embodiment comprises cyclosporin A, T-Det LF-416®, and Corn oil.

Another embodiment comprises cyclosporin A, Tergitol Min-Foam 1X®, and Corn oil.

Another embodiment comprises cyclosporin A, Tergitol Min-Foam 2X®, and Corn oil.

Another embodiment comprises cyclosporin A, Triton CF-21®, and Corn oil.

Another embodiment comprises cyclosporin A, Triton CF-76®, and Corn oil.

Another embodiment comprises cyclosporin A, Triton XL-80N®, and Corn oil.

Another embodiment comprises cyclosporin A, Witconol NS-98®, and Corn oil.

Another embodiment comprises cyclosporin A, Witconol NS-108LQ®, and Corn oil.

Another embodiment comprises cyclosporin A, Witconol NS-145®, and Corn oil.

Another embodiment comprises cyclosporin A, Witconol NS-179®, and Corn oil.

Another embodiment comprises cyclosporin A, a Protein-based Surfactant, and Corn oil.

Another embodiment comprises cyclosporin A, AminoFoam W®, and Corn oil.

Another embodiment comprises cyclosporin A, Amiter LGOD-2®, and Corn oil.

Another embodiment comprises cyclosporin A, Amiter LGS-2®, and Corn oil.

Another embodiment comprises cyclosporin A, Amiter LGS-5®, and Corn oil.

Another embodiment comprises cyclosporin A, Lamepon S®, and Corn oil.

Another embodiment comprises cyclosporin A, Lamepon S®, and Corn oil.

Another embodiment comprises cyclosporin A, Maypon 4C®, and Corn oil.

Another embodiment comprises cyclosporin A, May-Tein C®, and Corn oil.

Another embodiment comprises cyclosporin A, May-Tein CT®, and Corn oil.

Another embodiment comprises cyclosporin A, May-Tein KTS®, and Corn oil.

Another embodiment comprises cyclosporin A, May-Tein SY®, and Corn oil.

Another embodiment comprises cyclosporin A, Plantapon S®, and Corn oil.

Another embodiment comprises cyclosporin A, Proteol APL®, and Corn oil.

Another embodiment comprises cyclosporin A, Proteol OAT®, and Corn oil.

Another embodiment comprises cyclosporin A, Pyroter CPI-40®, and Corn oil.

Another embodiment comprises cyclosporin A, Pyroter GPI-25®, and Corn oil.

Another embodiment comprises cyclosporin A, Supro-Tein S®, and Corn oil.

Another embodiment comprises cyclosporin A, Supro-Tein V®, and Corn oil.

Another embodiment comprises cyclosporin A, a Sarcosine Derivative®, and Corn oil.

Another embodiment comprises cyclosporin A, Crodasinic LS-30®, and Com oil.

Another embodiment comprises cyclosporin A, Vanseal CS®, and Corn oil.

Another embodiment comprises cyclosporin A, Vanseal LS®, and Corn oil.

Another embodiment comprises cyclosporin A, Vanseal MS®, and Corn oil.

Another embodiment comprises cyclosporin A, Vanseal NACS-30®, and Corn oil.

Another embodiment comprises cyclosporin A, Vanseal NALS-95®, and Corn oil.

Another embodiment comprises cyclosporin A, Vanseal OS®, and Corn oil.

Another embodiment comprises cyclosporin A,® and Corn oil.

Another embodiment comprises cyclosporin A, a Silicone-based Surfactant®, and Corn oil.

Another embodiment comprises cyclosporin A, Abil-B-9950®, and Corn oil.

Another embodiment comprises cyclosporin A, Abil Care 85®, and Corn oil.

Another embodiment comprises cyclosporin A, Abil EM 90®, and Corn oil.

Another embodiment comprises cyclosporin A, Abil EM 97®, and Corn oil.

Another embodiment comprises cyclosporin A, Abil WE-09®, and Corn oil.

Another embodiment comprises cyclosporin A, Dow Corning 1248 Fluid®, and Corn oil.

Another embodiment comprises cyclosporin A, Dow Corning 3225C®, and Corn oil.

Another embodiment comprises cyclosporin A, Dow Corning 5200®, and Corn oil.

Another embodiment comprises cyclosporin A, Dow Corning 04-3667®, and Corn oil.

Another embodiment comprises cyclosporin A, Monasil PCA®, and Corn oil.

Another embodiment comprises cyclosporin A, Monasil PDM®, and Corn oil.

Another embodiment comprises cyclosporin A, Monasil PLN®, and Corn oil.

Another embodiment comprises cyclosporin A, Polyderm PPI-SI-WS®, and Corn oil.

Another embodiment comprises cyclosporin A, Troysol 380W®, and Corn oil.

Another embodiment comprises cyclosporin A, Troysol S366®, and Corn oil.

Another embodiment comprises cyclosporin A, a Sorbitan Derivative, and Corn oil.

Another embodiment comprises cyclosporin A, Alkamuls SML®, and Corn oil.

Another embodiment comprises cyclosporin A, Alkamuls SMO®, and Corn oil.

Another embodiment comprises cyclosporin A, Alkamuls STO®, and Corn oil.

Another embodiment comprises cyclosporin A, Arlacel 20®, and Corn oil.

Another embodiment comprises cyclosporin A, Arlacel 40®, and Corn oil.

Another embodiment comprises cyclosporin A, Arlacel 60®, and Corn oil.

Another embodiment comprises cyclosporin A, Arlacel 80®, and Corn oil.

Another embodiment comprises cyclosporin A, Arlacel C®, and Corn oil.

Another embodiment comprises cyclosporin A, Armul 21®, and Corn oil.

Another embodiment comprises cyclosporin A, Atlox 80®, and Corn oil.

Another embodiment comprises cyclosporin A, Atlox 847®, and Corn oil.

Another embodiment comprises cyclosporin A, Atlox 1045A®, and Corn oil.

Another embodiment comprises cyclosporin A, Canarcel 20®, and Corn oil.

Another embodiment comprises cyclosporin A, Canarcel 60®, and Corn oil.

Another embodiment comprises cyclosporin A, Canarcel 80®, and Corn oil.

Another embodiment comprises cyclosporin A, Canarcel TW 20®, and Corn oil.

Another embodiment comprises cyclosporin A, Canarcel TW 60®, and Corn oil.

Another embodiment comprises cyclosporin A, Canarcel TW 80®, and Corn oil.

Another embodiment comprises cyclosporin A, Coladet BSB-P®, and Corn oil.

Another embodiment comprises cyclosporin A, Customulse O-20®, and Corn oil.

Another embodiment comprises cyclosporin A, Dehymuls E®, and Corn oil.

Another embodiment comprises cyclosporin A, DeSotan SMO®, and Corn oil.

Another embodiment comprises cyclosporin A, DeSotan SMO-20®, and Corn oil.

Another embodiment comprises cyclosporin A, DeSotan SMT®, and Corn oil.

Another embodiment comprises cyclosporin A, DeSotan SMT-20®, and Corn oil.

Another embodiment comprises cyclosporin A, Durfax 60®, and Corn oil.

Another embodiment comprises cyclosporin A, Durfax 65®, and Corn oil.

Another embodiment comprises cyclosporin A, Durfax 80®, and Corn oil.

Another embodiment comprises cyclosporin A, Durtan 60®, and Corn oil.

Another embodiment comprises cyclosporin A, Durtan 65®, and Corn oil.

Another embodiment comprises cyclosporin A, Liposorb L®, and Corn oil.

Another embodiment comprises cyclosporin A, Liposorb L-10®, and Corn oil.

Another embodiment comprises cyclosporin A, Liposorb L-20®, and Corn oil.

Another embodiment comprises cyclosporin A, Liposorb C®, and Corn oil.

Another embodiment comprises cyclosporin A, Liposorb O-20®, and Corn oil.

Another embodiment comprises cyclosporin A, Liposorb P®, and Corn oil.

Another embodiment comprises cyclosporin A, Liposorb P-20®, and Corn oil.

Another embodiment comprises cyclosporin A, Liposorb S®, and Corn oil.

Another embodiment comprises cyclosporin A, Liposorb S-20®, and Corn oil.

Another embodiment comprises cyclosporin A, Liposorb SQO®, and Corn oil.

Another embodiment comprises cyclosporin A, Liposorb TO®, and Corn oil.

Another embodiment comprises cyclosporin A, Liposorb TS®, and Corn oil.

Another embodiment comprises cyclosporin A, Liposorb TS-20®, and Corn oil.

Another embodiment comprises cyclosporin A, Lumisorb PS®, and Corn oil.

Another embodiment comprises cyclosporin A, Lumisorb SMO (T)®, and Corn oil.

Another embodiment comprises cyclosporin A, Lumisorb SMS K®, and Corn oil.

Another embodiment comprises cyclosporin A, Lumisorb SSO®, and Corn oil.

Another embodiment comprises cyclosporin A, Lumisorb STS K®, and Corn oil.

Another embodiment comprises cyclosporin A, Lumisorb STT®, and Corn oil.

Another embodiment comprises cyclosporin A, Magrabar SMO®, and Corn oil.

Another embodiment comprises cyclosporin A, Magrabar SMO-VEG®, and Corn oil.

Another embodiment comprises cyclosporin A, Magrabar SMT®, and Corn oil.

Another embodiment comprises cyclosporin A, Magrabar STC®, and Corn oil.

Another embodiment comprises cyclosporin A, Miracare BC-27®, and Corn oil.

Another embodiment comprises cyclosporin A, Ritabate 20®, and Corn oil.

Another embodiment comprises cyclosporin A, Ritabate 40®, and Corn oil.

Another embodiment comprises cyclosporin A, Ritabate 60®, and Corn oil.

Another embodiment comprises cyclosporin A, Ritabate 80®, and Corn oil.

Another embodiment comprises cyclosporin A, T-Maz®, and Corn oil.

Another embodiment comprises cyclosporin A, Tego SML®, and Corn oil.

Another embodiment comprises cyclosporin A, Tego SML 20®, and Corn oil.

Another embodiment comprises cyclosporin A, Tego SMO 80 V®, and Corn oil.

Another embodiment comprises cyclosporin A, Tego SMO V®, and Corn oil.

Another embodiment comprises cyclosporin A, Tego SMS®, and Corn oil.

Another embodiment comprises cyclosporin A, Tego STO V®, and Corn oil.

Another embodiment comprises cyclosporin A, Tween 21®, and Corn oil.

Another embodiment comprises cyclosporin A, Tween 40®, and Corn oil.

Another embodiment comprises cyclosporin A, Tween 60®, and Corn oil.

Another embodiment comprises cyclosporin A, Tween 60 K®, and Corn oil.

Another embodiment comprises cyclosporin A, Tween 61®, and Corn oil.

Another embodiment comprises cyclosporin A, Tween 65®, and Corn oil.

Another embodiment comprises cyclosporin A, Tween 80®, and Corn oil.

Another embodiment comprises cyclosporin A, Tween 80 K®, and Corn oil.

Another embodiment comprises cyclosporin A, Tween 81®, and Corn oil.

Another embodiment comprises cyclosporin A, Tween 85®, and Corn oil.

Another embodiment comprises cyclosporin A, a Sucrose or Glucose Ester, or Derivative thereof, and Corn oil.

Another embodiment comprises cyclosporin A, DeSulf GOS-P-60WCG®, and Corn oil.

Another embodiment comprises cyclosporin A, Glucam E-20 Distearate®, and Corn oil.

Another embodiment comprises cyclosporin A, Glucamate DOE-120®, and Corn oil.

Another embodiment comprises cyclosporin A, Glucamate SSE-20®, and Corn oil.

Another embodiment comprises cyclosporin A, Glucate DO®, and Corn oil.

Another embodiment comprises cyclosporin A, Glucate SS®, and Corn oil.

Another embodiment comprises cyclosporin A, Glucopon 425 UP®, and Corn oil.

Another embodiment comprises cyclosporin A, isolan IS®, and Corn oil.

Another embodiment comprises cyclosporin A, Mazon 40®, and Corn oil.

Another embodiment comprises cyclosporin A, Montanov 82®, and Corn oil.

Another embodiment comprises cyclosporin A, Montanov 202®, and Corn oil.

Another embodiment comprises cyclosporin A, Montanov S®, and Corn oil.

Another embodiment comprises cyclosporin A, Rheozan®, and Corn oil.

Another embodiment comprises cyclosporin A, Simulsol AS 48®, and Corn oil.

Another embodiment comprises cyclosporin A, Simulsol SL 4®, and Corn oil.

Another embodiment comprises cyclosporin A, Simulsol SL 10®, and Corn oil.

Another embodiment comprises cyclosporin A, Simulsol SL 11W®, and Corn oil.

Another embodiment comprises cyclosporin A, Simulsol SL 55®, and Corn oil.

Another embodiment comprises cyclosporin A, Suga Nate 100 and 160®, and Corn oil.

Another embodiment comprises cyclosporin A, Tego Care 450®, and Corn oil.

Another embodiment comprises cyclosporin A, Tego Care CG 90®, and Corn oil.

Another embodiment comprises cyclosporin A, Tego Care PS®, and Corn oil.

Another embodiment comprises cyclosporin A, Tegosoft PSE 141 G®, and Corn oil.

Another embodiment comprises cyclosporin A, Tegotens G 826®, and Corn oil.

Another embodiment comprises cyclosporin A, Triton BG-10 (70%)®, and Corn oil.

Another embodiment comprises cyclosporin A, Triton CG-110 (60%)®, and Corn oil.

Another embodiment comprises cyclosporin A, Wickenol 545®, and Corn oil.

Another embodiment comprises cyclosporin A, an alcohol, and Arachis oil.

Another embodiment comprises cyclosporin A, Diglycerol®, and Arachis oil.

Another embodiment comprises cyclosporin A, Hetoxide GT-80®, and Arachis oil.

Another embodiment comprises cyclosporin A, Lexemul BEO®, and Arachis oil.

Another embodiment comprises cyclosporin A, Polyglycerol-3®, and Arachis oil.

Another embodiment comprises cyclosporin A, Redicote E Series®, and Arachis oil.

Another embodiment comprises cyclosporin A, Simulsol OX 1005L®, and Arachis oil.

Another embodiment comprises cyclosporin A, Stanfax 567®, and Arachis oil.

Another embodiment comprises cyclosporin A, TA-1618®, and Arachis oil.

Another embodiment comprises cyclosporin A, Witconol H-31A®, and Arachis oil.

Another embodiment comprises cyclosporin A, an amine oxide, and Arachis oil.

Another embodiment comprises cyclosporin A, AO-405®, and Arachis oil.

Another embodiment comprises cyclosporin A, AO-455®, and Arachis oil.

Another embodiment comprises cyclosporin A, AO 728 Special®, and Arachis oil.

Another embodiment comprises cyclosporin A, Barlox 12®, and Arachis oil.

Another embodiment comprises cyclosporin A, Barlox 14®, and Arachis oil.

Another embodiment comprises cyclosporin A, Burcoxide Lo®, and Arachis oil.

Another embodiment comprises cyclosporin A, Caloxamine LO®, and Arachis oil.

Another embodiment comprises cyclosporin A, Chemoxide CAW®, and Arachis oil.

Another embodiment comprises cyclosporin A, Chemoxide LM-30®, and Arachis oil.

Another embodiment comprises cyclosporin A, Chemoxide LO®, and Arachis oil.

Another embodiment comprises cyclosporin A, Chemoxide MO®, and Arachis oil.

Another embodiment comprises cyclosporin A, Colalux CAO-35®, and Arachis oil.

Another embodiment comprises cyclosporin A, Colalux LO®, and Arachis oil.

Another embodiment comprises cyclosporin A, DeMox CAPO®, and Arachis oil.

Another embodiment comprises cyclosporin A, DeMox CSG-30®, and Arachis oil.

Another embodiment comprises cyclosporin A, DeMox LAO®, and Arachis oil.

Another embodiment comprises cyclosporin A, Emcol L®, and Arachis oil.

Another embodiment comprises cyclosporin A, Empigen OB®, and Arachis oil.

Another embodiment comprises cyclosporin A, Empigen OS/A®, and Arachis oil.

Another embodiment comprises cyclosporin A, Foamox CDO®, and Arachis oil.

Another embodiment comprises cyclosporin A, Foamox DMM®, and Arachis oil.

Another embodiment comprises cyclosporin A, Foamox DMS®, and Arachis oil.

Another embodiment comprises cyclosporin A, Genaminox KC®, and Arachis oil.

Another embodiment comprises cyclosporin A, Genaminox LA®, and Arachis oil.

Another embodiment comprises cyclosporin A, Hartofoam SAO®, and Arachis oil.

Another embodiment comprises cyclosporin A, Hartox DMCD®, and Arachis oil.

Another embodiment comprises cyclosporin A, Lipowax DAT®, and Arachis oil.

Another embodiment comprises cyclosporin A, Lipowax PB Pastilles®, and Arachis oil.

Another embodiment comprises cyclosporin A, Mackamine C8®, and Arachis oil.

Another embodiment comprises cyclosporin A, Mackamine C10®, and Arachis oil.

Another embodiment comprises cyclosporin A, Mackamine C14®, and Arachis oil.

Another embodiment comprises cyclosporin A, Mackamine CAO®, and Arachis oil.

Another embodiment comprises cyclosporin A, Mackamine CO®, and Arachis oil.

Another embodiment comprises cyclosporin A, Mackamine LO®, and Arachis oil.

Another embodiment comprises cyclosporin A, Mackamine O2®, and Arachis oil.

Another embodiment comprises cyclosporin A, Mackamine SAO®, and Arachis oil.

Another embodiment comprises cyclosporin A, Mackamine SO®, and Arachis oil.

Another embodiment comprises cyclosporin A, Mazox KCAO®, and Arachis oil.

Another embodiment comprises cyclosporin A, Monalac MO®, and Arachis oil.

Another embodiment comprises cyclosporin A, Norfox LDA®, and Arachis oil.

Another embodiment comprises cyclosporin A, Rhodamox LO®, and Arachis oil.

Another embodiment comprises cyclosporin A, Schercamox C-AA®, and Arachis oil.

Another embodiment comprises cyclosporin A, Schercamox DMA®, and Arachis oil.

Another embodiment comprises cyclosporin A, Schercamox DML®, and Arachis oil.

Another embodiment comprises cyclosporin A, Schercamox DMM®, and Arachis oil.

Another embodiment comprises cyclosporin A, Schercamox DMS®, and Arachis oil.

Another embodiment comprises cyclosporin A, Tegotens DO®, and Arachis oil.

Another embodiment comprises cyclosporin A, Tomah AO-14-2®, and Arachis oil.

Another embodiment comprises cyclosporin A, Triaminox CDO®, and Arachis oil.

Another embodiment comprises cyclosporin A, a block polymer, and Arachis oil.

Another embodiment comprises cyclosporin A, AL 2070®, and Arachis oil.

Another embodiment comprises cyclosporin A, Antarox 17-R-2®, and Arachis oil.

Another embodiment comprises cyclosporin A, Antarox 25-R-2®, and Arachis oil.

Another embodiment comprises cyclosporin A, Antarox 31-R-1®, and Arachis oil.

Another embodiment comprises cyclosporin A, Antarox P-84®, and Arachis oil.

Another embodiment comprises cyclosporin A, Antarox P-104/H®, and Arachis oil.

Another embodiment comprises cyclosporin A, Arnox BP-Series®, and Arachis oil.

Another embodiment comprises cyclosporin A, Chemonic 435®, and Arachis oil.

Another embodiment comprises cyclosporin A, Chemonic D-25®, and Arachis oil.

Another embodiment comprises cyclosporin A, Chemonic PL Series®, and Arachis oil.

Another embodiment comprises cyclosporin A, Ethox L-121®, and Arachis oil.

Another embodiment comprises cyclosporin A, Ethox L-122®, and Arachis oil.

Another embodiment comprises cyclosporin A, Genapol PF-10®, and Arachis oil.

Another embodiment comprises cyclosporin A, Genapol PF-20®, and Arachis oil.

Another embodiment comprises cyclosporin A, Genapol PF-40A®, and Arachis oil.

Another embodiment comprises cyclosporin A, Norfox 2-LF®, and Arachis oil.

Another embodiment comprises cyclosporin A, Simulsol NW 342®, and Arachis oil.

Another embodiment comprises cyclosporin A, T-Det BP-1®, and Arachis oil.

Another embodiment comprises cyclosporin A, T-Det XD®, and Arachis oil.

Another embodiment comprises cyclosporin A, T-Det XH®, and Arachis oil.

Another embodiment comprises cyclosporin A, Triton CF-32®, and Arachis oil.

Another embodiment comprises cyclosporin A, Witconol 171®, and Arachis oil.

Another embodiment comprises cyclosporin A, Witconol 324®, and Arachis oil.

Another embodiment comprises cyclosporin A, Witconol 324D®, and Arachis oil.

Another embodiment comprises cyclosporin A, Witconol PD-2000®, and Arachis oil.

Another embodiment comprises cyclosporin A, a Carboxylated Alcohol or Alkylphenol Ethoxylate, and Arachis oil.

Another embodiment comprises cyclosporin A, Emcol CN-6®, and Arachis oil.

Another embodiment comprises cyclosporin A, Ethcarb®, and Arachis oil.

Another embodiment comprises cyclosporin A, Gemtex WNT-Conc®, and Arachis oil.

Another embodiment comprises cyclosporin A, Incrodet TD7-C®, and Arachis oil.

Another embodiment comprises cyclosporin A, Marlinat CM 105/80®, and Arachis oil.

Another embodiment comprises cyclosporin A, Marlowet 1072®, and Arachis oil.

Another embodiment comprises cyclosporin A, Marlowet 4530®, and Arachis oil.

Another embodiment comprises cyclosporin A, Marlowet 4530 LF®, and Arachis oil.

Another embodiment comprises cyclosporin A, Marlowet 4534®, and Arachis oil.

Another embodiment comprises cyclosporin A, Marlowet 4538®, and Arachis oil.

Another embodiment comprises cyclosporin A, Marlowet 4539®, and Arachis oil.

Another embodiment comprises cyclosporin A, Marlowet 4539 LF®, and Arachis oil.

Another embodiment comprises cyclosporin A, Marlowet 4541®, and Arachis oil.

Another embodiment comprises cyclosporin A, Miranate LEC-80®, and Arachis oil.

Another embodiment comprises cyclosporin A, Sandopan B®, and Arachis oil.

Another embodiment comprises cyclosporin A, Sandopan B Modified®, and Arachis oil.

Another embodiment comprises cyclosporin A, Sandopan LS-24 Gel®, and Arachis oil.

Another embodiment comprises cyclosporin A, Surfine T-A®, and Arachis oil.

Another embodiment comprises cyclosporin A, a Carboxylic Acid/Fatty Acid, and Arachis oil.

Another embodiment comprises cyclosporin A, Colaterge RAM®, and Arachis oil.

Another embodiment comprises cyclosporin A, Colatrope INC®, and Arachis oil.

Another embodiment comprises cyclosporin A, Crodacid B®, and Arachis oil.

Another embodiment comprises cyclosporin A, DeTrope CA-100®, and Arachis oil.

Another embodiment comprises cyclosporin A, Latol MTO®, and Arachis oil.

Another embodiment comprises cyclosporin A, Lumulse CC-33 K®, and Arachis oil.

Another embodiment comprises cyclosporin A, Mulls 2218®, and Arachis oil.

Another embodiment comprises cyclosporin A, OL-600®, and Arachis oil.

Another embodiment comprises cyclosporin A, OL-800®, and Arachis oil.

Another embodiment comprises cyclosporin A, R-910®, and Arachis oil.

Another embodiment comprises cyclosporin A, S-210®, and Arachis oil.

Another embodiment comprises cyclosporin A, Sandopan DTC Acid®, and Arachis oil.

Another embodiment comprises cyclosporin A, Sandopan LS 24 N®, and Arachis oil.

Another embodiment comprises cyclosporin A, Sandopan MA-18®, and Arachis oil.

Another embodiment comprises cyclosporin A, an Ethoxylated Alcohol, and Arachis oil.

Another embodiment comprises cyclosporin A, Adsee 799®, and Arachis oil.

Another embodiment comprises cyclosporin A, Adsee 799®, and Arachis oil.

Another embodiment comprises cyclosporin A, Alfonic 610-3.5®, and Arachis oil.

Another embodiment comprises cyclosporin A, Alfonic 810-2®, and Arachis oil.

Another embodiment comprises cyclosporin A, Alfonic 810-6®, and Arachis oil.

Another embodiment comprises cyclosporin A, Alfonic 1012-3®, and Arachis oil.

Another embodiment comprises cyclosporin A, Alfonic 1012-5®, and Arachis oil.

Another embodiment comprises cyclosporin A, Alfonic 1216CO-1.5®, and Arachis oil.

Another embodiment comprises cyclosporin A, Alfonic 1216CO-7®, and Arachis oil.

Another embodiment comprises cyclosporin A, Alfonic 1412-3®, and Arachis oil.

Another embodiment comprises cyclosporin A, Alfonic 1412-7®, and Arachis oil.

Another embodiment comprises cyclosporin A, Arlasolve 200®, and Arachis oil.

Another embodiment comprises cyclosporin A, Arlasolve 200 Liquid®, and Arachis oil.

Another embodiment comprises cyclosporin A, Armix 180-C®, and Arachis oil.

Another embodiment comprises cyclosporin A, Armix 183®, and Arachis oil.

Another embodiment comprises cyclosporin A, Armul 2404®, and Arachis oil.

Another embodiment comprises cyclosporin A, Armul 2404®, and Arachis oil.

Another embodiment comprises cyclosporin A, Atlas EMJ-C®, and Arachis oil.

Another embodiment comprises cyclosporin A, Atlas G-2109®, and Arachis oil.

Another embodiment comprises cyclosporin A, Atlas G-3886®, and Arachis oil.

Another embodiment comprises cyclosporin A, Atlas G-3890®, and Arachis oil.

Another embodiment comprises cyclosporin A, Bio Soft E-200®, and Arachis oil.

Another embodiment comprises cyclosporin A, Bio Soft E-300®, and Arachis oil.

Another embodiment comprises cyclosporin A, Bio Soft E-400®, and Arachis oil.

Another embodiment comprises cyclosporin A, Bio Soft EN 600®, and Arachis oil.

Another embodiment comprises cyclosporin A, Bio Soft TD-400®, and Arachis oil.

Another embodiment comprises cyclosporin A, Bio Soft TD-630®, and Arachis oil.

Another embodiment comprises cyclosporin A, Brij 30®, and Arachis oil.

Another embodiment comprises cyclosporin A, Brij 52®, and Arachis oil.

Another embodiment comprises cyclosporin A, Brij 56®, and Arachis oil.

Another embodiment comprises cyclosporin A, Brij 58®, and Arachis oil.

Another embodiment comprises cyclosporin A, Brij 72®, and Arachis oil.

Another embodiment comprises cyclosporin A, Brij 76®, and Arachis oil.

Another embodiment comprises cyclosporin A, Brij 78®, and Arachis oil.

Another embodiment comprises cyclosporin A, Brij 93®, and Arachis oil.

Another embodiment comprises cyclosporin A, Brij 97®, and Arachis oil.

Another embodiment comprises cyclosporin A, Brij 98®, and Arachis oil.

Another embodiment comprises cyclosporin A, Brij 700®, and Arachis oil.

Another embodiment comprises cyclosporin A, Brij 700 S®, and Arachis oil.

Another embodiment comprises cyclosporin A, Brij 721®, and Arachis oil.

Another embodiment comprises cyclosporin A, Brij 721 S®, and Arachis oil.

Another embodiment comprises cyclosporin A, Burcoterge CDG®, and Arachis oil.

Another embodiment comprises cyclosporin A, Canasol AT 600®, and Arachis oil.

Another embodiment comprises cyclosporin A, Canasol AT 800®, and Arachis oil.

Another embodiment comprises cyclosporin A, Canasol AT 1200®, and Arachis oil.

Another embodiment comprises cyclosporin A, Canasol BJ 35®, and Arachis oil.

Another embodiment comprises cyclosporin A, Canasol BJ 36®, and Arachis oil.

Another embodiment comprises cyclosporin A, Canasol BJ 52®, and Arachis oil.

Another embodiment comprises cyclosporin A, Canasol BJ 58®, and Arachis oil.

Another embodiment comprises cyclosporin A, Canasol BJ 72®, and Arachis oil.

Another embodiment comprises cyclosporin A, Canasol BJ 78®, and Arachis oil.

Another embodiment comprises cyclosporin A, Canasol BJ 98®, and Arachis oil.

Another embodiment comprises cyclosporin A, Canasol BJ 307®, and Arachis oil.

Another embodiment comprises cyclosporin A, Cerfak 1400®, and Arachis oil.

Another embodiment comprises cyclosporin A, Cetomacrogol 1000 BF®, and Arachis oil.

Another embodiment comprises cyclosporin A, Chemonic C-2®, and Arachis oil.

Another embodiment comprises cyclosporin A, Chemonic C-10®, and Arachis oil.

Another embodiment comprises cyclosporin A, Chemonic C-20®, and Arachis oil.

Another embodiment comprises cyclosporin A, Chemonic CT-12®, and Arachis oil.

Another embodiment comprises cyclosporin A, Chemonic CT-20®, and Arachis oil.

Another embodiment comprises cyclosporin A, Chemonic CT-30®, and Arachis oil.

Another embodiment comprises cyclosporin A, Chemonic CT-55®, and Arachis oil.

Another embodiment comprises cyclosporin A, Chemonic G-7®, and Arachis oil.

Another embodiment comprises cyclosporin A, Chemonic G-26®, and Arachis oil.

Another embodiment comprises cyclosporin A, Chemonic L-4®, and Arachis oil.

Another embodiment comprises cyclosporin A, Chemonic L-7®, and Arachis oil.

Another embodiment comprises cyclosporin A, Chemonic L-12®, and Arachis oil.

Another embodiment comprises cyclosporin A, Chemonic L-23®, and Arachis oil.

Another embodiment comprises cyclosporin A, Chemonic O-2®, and Arachis oil.

Another embodiment comprises cyclosporin A, Chemonic O-5®, and Arachis oil.

Another embodiment comprises cyclosporin A, Chemonic O-10®, and Arachis oil.

Another embodiment comprises cyclosporin A, Chemonic O-20®, and Arachis oil.

Another embodiment comprises cyclosporin A, Chemonic S-2®, and Arachis oil.

Another embodiment comprises cyclosporin A, Chemonic S-10®, and Arachis oil.

Another embodiment comprises cyclosporin A, Chemonic S-20®, and Arachis oil.

Another embodiment comprises cyclosporin A, Colamulse FE®, and Arachis oil.

Another embodiment comprises cyclosporin A, Cremophor A 20®, and Arachis oil.

Another embodiment comprises cyclosporin A, Cremophor SA 2®, and Arachis oil.

Another embodiment comprises cyclosporin A, Dehydol 100®, and Arachis oil.

Another embodiment comprises cyclosporin A, Dehydol O-4®, and Arachis oil.

Another embodiment comprises cyclosporin A, Delonic C-18®, and Arachis oil.

Another embodiment comprises cyclosporin A, DeSonic 6T®, and Arachis oil.

Another embodiment comprises cyclosporin A, DeSonic 9D®, and Arachis oil.

Another embodiment comprises cyclosporin A, DeSonic 9T®, and Arachis oil.

Another embodiment comprises cyclosporin A, DeSonic 12®, and Arachis oil.

Another embodiment comprises cyclosporin A, DeSonic 12®, and Arachis oil.

Another embodiment comprises cyclosporin A, DeSonic 15T®, and Arachis oil.

Another embodiment comprises cyclosporin A, DeSonic TDA-9®, and Arachis oil.

Another embodiment comprises cyclosporin A, DeThox GLG-7®, and Arachis oil.

Another embodiment comprises cyclosporin A, DeThox GLG-26®, and Arachis oil.

Another embodiment comprises cyclosporin A, DeThox LA-4®, and Arachis oil.

Another embodiment comprises cyclosporin A, DeThox LA-23®, and Arachis oil.

Another embodiment comprises cyclosporin A, DeThox SA-80®, and Arachis oil.

Another embodiment comprises cyclosporin A, Disponil 05®, and Arachis oil.

Another embodiment comprises cyclosporin A, Eccoterge EO-41 Be, and Arachis oil.

Another embodiment comprises cyclosporin A, Empilan KA2.5/90®, and Arachis oil.

Another embodiment comprises cyclosporin A, Empilan KA5/90®, and Arachis oil.

Another embodiment comprises cyclosporin A, Empilan KM-20®, and Arachis oil.

Another embodiment comprises cyclosporin A, Empilan KM-50®, and Arachis oil.

Another embodiment comprises cyclosporin A, Empilan L-23®, and Arachis oil.

Another embodiment comprises cyclosporin A, Ethylan 25-3®, and Arachis oil.

Another embodiment comprises cyclosporin A, Ethylan 1204®, and Arachis oil.

Another embodiment comprises cyclosporin A, Ethylan DA-4®, and Arachis oil.

Another embodiment comprises cyclosporin A, Ethylan LA-230®, and Arachis oil.

Another embodiment comprises cyclosporin A, Ethylan SN®, and Arachis oil.

Another embodiment comprises cyclosporin A, Ethylan TD-60®, and Arachis oil.

Another embodiment comprises cyclosporin A, Ethylan TD-10®, and Arachis oil.

Another embodiment comprises cyclosporin A, Ethylan TD-1407®, and Arachis oil.

Another embodiment comprises cyclosporin A, Eumulgin B1®, and Arachis oil.

Another embodiment comprises cyclosporin A, Eumulgin B2®, and Arachis oil.

Another embodiment comprises cyclosporin A, Eumulgin B3®, and Arachis oil.

Another embodiment comprises cyclosporin A, Eumulgin O-10®, and Arachis oil.

Another embodiment comprises cyclosporin A, Flo Mo 80/20®, and Arachis oil.

Another embodiment comprises cyclosporin A, Flo Mo Low Foam®, and Arachis oil.

Another embodiment comprises cyclosporin A, Forlan C-24®, and Arachis oil.

Another embodiment comprises cyclosporin A, Genapol 1454®, and Arachis oil.

Another embodiment comprises cyclosporin A, Genapol BA-020®, and Arachis oil.

Another embodiment comprises cyclosporin A, Genapol BA-040®, and Arachis oil.

Another embodiment comprises cyclosporin A, Genapol C-100®, and Arachis oil.

Another embodiment comprises cyclosporin A, Genapol DA 060®, and Arachis oil.

Another embodiment comprises cyclosporin A, Genapol HS 020®, and Arachis oil.

Another embodiment comprises cyclosporin A, Genapol HS 200®, and Arachis oil.

Another embodiment comprises cyclosporin A, Genapol ID-040®, and Arachis oil.

Another embodiment comprises cyclosporin A, Genapol ID-060®, and Arachis oil.

Another embodiment comprises cyclosporin A, Genapol ID-090®, and Arachis oil.

Another embodiment comprises cyclosporin A, Genapol LA 010®, and Arachis oil.

Another embodiment comprises cyclosporin A, Genapol LA 020®, and Arachis oil.

Another embodiment comprises cyclosporin A, Genapol LA 030®, and Arachis oil.

Another embodiment comprises cyclosporin A, Genapol LA 040®, and Arachis oil.

Another embodiment comprises cyclosporin A, Genapol LA 050®, and Arachis oil.

Another embodiment comprises cyclosporin A, Genapol LA 060®, and Arachis oil.

Another embodiment comprises cyclosporin A, Genapol LA 070®, and Arachis oil.

Another embodiment comprises cyclosporin A, Genapol LA 070S®, and Arachis oil.

Another embodiment comprises cyclosporin A, Genapol LA 230®, and Arachis oil.

Another embodiment comprises cyclosporin A, Genapol O 020®, and Arachis oil.

Another embodiment comprises cyclosporin A, Genapol O 050®, and Arachis oil.

Another embodiment comprises cyclosporin A, Genapol O 100®, and Arachis oil.

Another embodiment comprises cyclosporin A, Genapol O 200®, and Arachis oil.

Another embodiment comprises cyclosporin A, Genapol SA 030®, and Arachis oil.

Another embodiment comprises cyclosporin A, Genapol SA 120®, and Arachis oil.

Another embodiment comprises cyclosporin A, Genapol T-020®, and Arachis oil.

Another embodiment comprises cyclosporin A, Genapol UD-030®, and Arachis oil.

Another embodiment comprises cyclosporin A, Genapol UD-050®, and Arachis oil.

Another embodiment comprises cyclosporin A, Genapol UD-070®, and Arachis oil.

Another embodiment comprises cyclosporin A, Genapol UD-079®, and Arachis oil.

Another embodiment comprises cyclosporin A, Genapol UD-080®, and Arachis oil.

Another embodiment comprises cyclosporin A, Genapol UD-110®, and Arachis oil.

Another embodiment comprises cyclosporin A, Genapol X 030®, and Arachis oil.

Another embodiment comprises cyclosporin A, Genapol X 050®, and Arachis oil.

Another embodiment comprises cyclosporin A, Genapol X 060®, and Arachis oil.

Another embodiment comprises cyclosporin A, Genapol X 070®, and Arachis oil.

Another embodiment comprises cyclosporin A, Genapol X 080®, and Arachis oil.

Another embodiment comprises cyclosporin A, Genapol X 100®, and Arachis oil.

Another embodiment comprises cyclosporin A, Genapol X 159®, and Arachis oil.

Another embodiment comprises cyclosporin A, Generol 122 E5®, and Arachis oil.

Another embodiment comprises cyclosporin A, Generol 122 E25®, and Arachis oil.

Another embodiment comprises cyclosporin A, Hostacerin T-3®, and Arachis oil.

Another embodiment comprises cyclosporin A, Iconol LF 110®, and Arachis oil.

Another embodiment comprises cyclosporin A, Incropol CS-20®, and Arachis oil.

Another embodiment comprises cyclosporin A, Lexemul CS-20®, and Arachis oil.

Another embodiment comprises cyclosporin A, Liponic EG-1®, and Arachis oil.

Another embodiment comprises cyclosporin A, Lipowax D®, and Arachis oil.

Another embodiment comprises cyclosporin A, Lipowax G®, and Arachis oil.

Another embodiment comprises cyclosporin A, Lipowax NI®, and Arachis oil.

Another embodiment comprises cyclosporin A, Lipowax P®, and Arachis oil.

Another embodiment comprises cyclosporin A, Lipowax P-31®, and Arachis oil.

Another embodiment comprises cyclosporin A, Lipowax PR®, and Arachis oil.

Another embodiment comprises cyclosporin A, Lumulse CS-20®, and Arachis oil.

Another embodiment comprises cyclosporin A, Macol CSA-20®, and Arachis oil.

Another embodiment comprises cyclosporin A, Marlox B 24/50®, and Arachis oil.

Another embodiment comprises cyclosporin A, Mazawet 77®, and Arachis oil.

Another embodiment comprises cyclosporin A, Norfox 1713®, and Arachis oil.

Another embodiment comprises cyclosporin A, Norfox 2579®, and Arachis oil.

Another embodiment comprises cyclosporin A, Norfox Lo Foam®, and Arachis oil.

Another embodiment comprises cyclosporin A, Promulgen C®, and Arachis oil.

Another embodiment comprises cyclosporin A, Promulgen G®, and Arachis oil.

Another embodiment comprises cyclosporin A, Renex 30®, and Arachis oil.

Another embodiment comprises cyclosporin A, Renex 36®, and Arachis oil.

Another embodiment comprises cyclosporin A, Rhodasurf A 24®, and Arachis oil.

Another embodiment comprises cyclosporin A, Rhodasurf ME-10®, and Arachis oil.

Another embodiment comprises cyclosporin A, Rhodasurf BEH-25®, and Arachis oil.

Another embodiment comprises cyclosporin A, Rhodasurf BEH-40®, and Arachis oil.

Another embodiment comprises cyclosporin A, Rhodasurf DA 530®, and Arachis oil.

Another embodiment comprises cyclosporin A, Rhodasurf DA 630®, and Arachis oil.

Another embodiment comprises cyclosporin A, Rhodasurf DA 639®, and Arachis oil.

Another embodiment comprises cyclosporin A, Rhodasurf LAN-23®, and Arachis oil.

Another embodiment comprises cyclosporin A, Rhodasurf ON-870®, and Arachis oil.

Another embodiment comprises cyclosporin A, Rhodasurf ON-877®, and Arachis oil.

Another embodiment comprises cyclosporin A, Rhodasurf TB-970 FLK®, and Arachis oil.

Another embodiment comprises cyclosporin A, Ritacet-20®, and Arachis oil.

Another embodiment comprises cyclosporin A, Ritachol 1000®, and Arachis oil.

Another embodiment comprises cyclosporin A, Ritachol 2000®, and Arachis oil.

Another embodiment comprises cyclosporin A, Ritachol 5000®, and Arachis oil.

Another embodiment comprises cyclosporin A, Ritox 35®, and Arachis oil.

Another embodiment comprises cyclosporin A, Surfonic DA-4®, and Arachis oil.

Another embodiment comprises cyclosporin A, Surfonic DA-6®, and Arachis oil.

Another embodiment comprises cyclosporin A, Sunonic L46-7®, and Arachis oil.

Another embodiment comprises cyclosporin A, Surfonic POA®, and Arachis oil.

Another embodiment comprises cyclosporin A, Synthrapol KB®, and Arachis oil.

Another embodiment comprises cyclosporin A, Teginacid®, and Arachis oil.

Another embodiment comprises cyclosporin A, Teginacid C®, and Arachis oil.

Another embodiment comprises cyclosporin A, Tegotens EC 11®, and Arachis oil.

Another embodiment comprises cyclosporin A, Tinegal NA®, and Arachis oil.

Another embodiment comprises cyclosporin A, Tomadol 400®, and Arachis oil.

Another embodiment comprises cyclosporin A, Tomadol 600®, and Arachis oil.

Another embodiment comprises cyclosporin A, Tomadol 900®, and Arachis oil.

Another embodiment comprises cyclosporin A, Uniperol O®, and Arachis oil.

Another embodiment comprises cyclosporin A, Witconol SN Series®, and Arachis oil.

Another embodiment comprises cyclosporin A, an Ethoxylated Alkylphenol, and Arachis oil.

Another embodiment comprises cyclosporin A, Antarox LF-222®, and Arachis oil.

Another embodiment comprises cyclosporin A, Atlox 775®, and Arachis oil.

Another embodiment comprises cyclosporin A, Caloxylate N-9®, and Arachis oil.

Another embodiment comprises cyclosporin A, Canasol NF-1000®, and Arachis oil.

Another embodiment comprises cyclosporin A, Canasol NF-3000®, and Arachis oil.

Another embodiment comprises cyclosporin A, Canasol NF-3070®, and Arachis oil.

Another embodiment comprises cyclosporin A, Canasol OF 1670®, and Arachis oil.

Another embodiment comprises cyclosporin A, Canasol OF 2570®, and Arachis oil.

Another embodiment comprises cyclosporin A, Canasol OF 4070®, and Arachis oil.

Another embodiment comprises cyclosporin A, Chemax DNP-8®, and Arachis oil.

Another embodiment comprises cyclosporin A, Chemax DNP-18®, and Arachis oil.

Another embodiment comprises cyclosporin A, Chemax DNP-150/50®, and Arachis oil.

Another embodiment comprises cyclosporin A, DeSonic 1.5N®, and Arachis oil.

Another embodiment comprises cyclosporin A, DeSonic 4N®, and Arachis oil.

Another embodiment comprises cyclosporin A, DeSonic 5N®, and Arachis oil.

Another embodiment comprises cyclosporin A, DeSonic 6D®, and Arachis oil.

Another embodiment comprises cyclosporin A, DeSonic 6N®, and Arachis oil.

Another embodiment comprises cyclosporin A, DeSonic 7N®, and Arachis oil.

Another embodiment comprises cyclosporin A, DeSonic 9N®, and Arachis oil.

Another embodiment comprises cyclosporin A, DeSonic 10D®, and Arachis oil.

Another embodiment comprises cyclosporin A, DeSonic 11N®, and Arachis oil.

Another embodiment comprises cyclosporin A, DeSonic 12N®, and Arachis oil.

Another embodiment comprises cyclosporin A, DeSonic 15N®, and Arachis oil.

Another embodiment comprises cyclosporin A, DeSonic 20N®, and Arachis oil.

Another embodiment comprises cyclosporin A, Eccoscour RC®, and Arachis oil.

Another embodiment comprises cyclosporin A, Eccoterge EO-100®, and Arachis oil.

Another embodiment comprises cyclosporin A, Emulsifier 632/90%®, and Arachis oil.

Another embodiment comprises cyclosporin A, Geronol AG-821®, and Arachis oil.

Another embodiment comprises cyclosporin A, Gradonic N-95®, and Arachis oil.

Another embodiment comprises cyclosporin A, Hetoxide NP-4®, and Arachis oil.

Another embodiment comprises cyclosporin A, Hetoxide NP-30®, and Arachis oil.

Another embodiment comprises cyclosporin A, Hostapal N-100®, and Arachis oil.

Another embodiment comprises cyclosporin A, Hostapal N-110®, and Arachis oil.

Another embodiment comprises cyclosporin A, Igepal CTA-639W®, and Arachis oil.

Another embodiment comprises cyclosporin A, Igepal DAP-9®, and Arachis oil.

Another embodiment comprises cyclosporin A, Igepal OD-410®, and Arachis oil.

Another embodiment comprises cyclosporin A, Igepal SS-837®, and Arachis oil.

Another embodiment comprises cyclosporin A, Lipocol NP-9 USP®, and Arachis oil.

Another embodiment comprises cyclosporin A, Macol DNP-10®, and Arachis oil.

Another embodiment comprises cyclosporin A, Marlophen NP 5®, and Arachis oil.

Another embodiment comprises cyclosporin A, Marlophen P 1®, and Arachis oil.

Another embodiment comprises cyclosporin A, Surfonic NB®, and Arachis oil.

Another embodiment comprises cyclosporin A, Surfonic OPB-307®, and Arachis oil.

Another embodiment comprises cyclosporin A, Surfonic OPB-407®, and Arachis oil.

Another embodiment comprises cyclosporin A, Syn Fac 334®, and Arachis oil.

Another embodiment comprises cyclosporin A, Syn Fac 8216®, and Arachis oil.

Another embodiment comprises cyclosporin A, Triton N-57®, and Arachis oil.

Another embodiment comprises cyclosporin A, Trycol 6956®, and Arachis oil.

Another embodiment comprises cyclosporin A, Trycol 6961®, and Arachis oil.

Another embodiment comprises cyclosporin A, Trycol 6964®, and Arachis oil.

Another embodiment comprises cyclosporin A, Trycol 6969®, and Arachis oil.

Another embodiment comprises cyclosporin A, Trycol 6974®, and Arachis oil.

Another embodiment comprises cyclosporin A, Witbreak DRB-127®, and Arachis oil.

Another embodiment comprises cyclosporin A, Witbreak DRB-127®, and Arachis oil.

Another embodiment comprises cyclosporin A, Witconol NP Series®, and Arachis oil.

Another embodiment comprises cyclosporin A, an Ethoxylated Aryl Phenol, and Arachis oil.

Another embodiment comprises cyclosporin A, Soprophor BSU®, and Arachis oil.

Another embodiment comprises cyclosporin A, Soprophor CY/8®, and Arachis oil.

Another embodiment comprises cyclosporin A, Soprophor S/25®, and Arachis oil.

Another embodiment comprises cyclosporin A, Witconol NIO®, and Arachis oil.

Another embodiment comprises cyclosporin A, Witconol NIW®, and Arachis oil.

Another embodiment comprises cyclosporin A, Witconol S-100®, and Arachis oil.

Another embodiment comprises cyclosporin A, an Ethoxylated Fatty Acid. and Arachis oil.

Another embodiment comprises cyclosporin A, Aldo PGHMS®, and Arachis oil.

Another embodiment comprises cyclosporin A, Alkamuls TO-15/HR®, and Arachis oil.

Another embodiment comprises cyclosporin A, Armotan AL-69-66®, and Arachis oil.

Another embodiment comprises cyclosporin A, Cerasynt 840®, and Arachis oil.

Another embodiment comprises cyclosporin A, Cerasynt 945®, and Arachis oil.

Another embodiment comprises cyclosporin A, Crystal Inhibitor No. 5®, and Arachis oil.

Another embodiment comprises cyclosporin A, DeThox Acid L-9®, and Arachis oil.

Another embodiment comprises cyclosporin A, DeThox Acid S-8®, and Arachis oil.

Another embodiment comprises cyclosporin A, Ethofat 242/25®, and Arachis oil.

Another embodiment comprises cyclosporin A, Hydropalat 65®, and Arachis oil.

Another embodiment comprises cyclosporin A, Lipo EGMS®, and Arachis oil.

Another embodiment comprises cyclosporin A, Lipopeg 2 DL®, and Arachis oil.

Another embodiment comprises cyclosporin A, Lipopeg 4 DL®, and Arachis oil.

Another embodiment comprises cyclosporin A, Lipopeg 4-L®, and Arachis oil.

Another embodiment comprises cyclosporin A, Lipopeg 39-S®, and Arachis oil.

Another embodiment comprises cyclosporin A, Lipopeg 4-S®, and Arachis oil.

Another embodiment comprises cyclosporin A, Lipopeg 10-S®, and Arachis oil.

Another embodiment comprises cyclosporin A, Lipopeg 100-S®, and Arachis oil.

Another embodiment comprises cyclosporin A, Lipopeg 6000 DS®, and Arachis oil.

Another embodiment comprises cyclosporin A, Lumulse 40-L®, and Arachis oil.

Another embodiment comprises cyclosporin A, Lumulse 40-S®, and Arachis oil.

Another embodiment comprises cyclosporin A, Lumulse 42-L®, and Arachis oil.

Another embodiment comprises cyclosporin A, Lumulse 42-S®, and Arachis oil.

Another embodiment comprises cyclosporin A, Lumulse 100-S®, and Arachis oil.

Another embodiment comprises cyclosporin A, Lumulse 602-S®, and Arachis oil.

Another embodiment comprises cyclosporin A, Magrabar PGE-20-0®, and Arachis oil.

Another embodiment comprises cyclosporin A, Magrabar PGE-20L®, and Arachis oil.

Another embodiment comprises cyclosporin A, Magrabar PGE-20T®, and Arachis oil.

Another embodiment comprises cyclosporin A, Magrabar PGE-22-0®, and Arachis oil.

Another embodiment comprises cyclosporin A, Magrabar PGE-22L®, and Arachis oil.

Another embodiment comprises cyclosporin A, Magrabar PGE-22T®, and Arachis oil.

Another embodiment comprises cyclosporin A, Magrabar PGE-40-0®, and Arachis oil.

Another embodiment comprises cyclosporin A, Magrabar PGE-40L®, and Arachis oil.

Another embodiment comprises cyclosporin A, Magrabar PGE-40T®, and Arachis oil.

Another embodiment comprises cyclosporin A, Magrabar PGE-42-0®, and Arachis oil.

Another embodiment comprises cyclosporin A, Magrabar PGE-42L®, and Arachis oil.

Another embodiment comprises cyclosporin A, Magrabar PGE-42T®, and Arachis oil.

Another embodiment comprises cyclosporin A, Magrabar PGE-60-0®, and Arachis oil.

Another embodiment comprises cyclosporin A, Magrabar PGE-60L®, and Arachis oil.

Another embodiment comprises cyclosporin A, Magrabar PGE-60T®, and Arachis oil.

Another embodiment comprises cyclosporin A, Magrabar PGE-62-0®, and Arachis oil.

Another embodiment comprises cyclosporin A, Magrabar PGE-62L®, and Arachis oil.

Another embodiment comprises cyclosporin A, Magrabar PGE-62T®, and Arachis oil.

Another embodiment comprises cyclosporin A, Mapeg S-40K®, and Arachis oil.

Another embodiment comprises cyclosporin A, Marlowet OTS®, and Arachis oil.

Another embodiment comprises cyclosporin A, Naturechem PGR®, and Arachis oil.

Another embodiment comprises cyclosporin A, PG No. 4®, and Arachis oil.

Another embodiment comprises cyclosporin A, Renex 20®, and Arachis oil.

Another embodiment comprises cyclosporin A, Ritox 52®, and Arachis oil;

Another embodiment comprises cyclosporin A, Ritox 53®, and Arachis oil.

Another embodiment comprises cyclosporin A, Ritox 59®, and Arachis oil.

Another embodiment comprises cyclosporin A, Surfax 8916/A®, and Arachis oil.

Another embodiment comprises cyclosporin A, Tego Acid S 40 P®, and Arachis oil.

Another embodiment comprises cyclosporin A, Tego Acid S 100 P®, and Arachis oil.

Another embodiment comprises cyclosporin A, Tween 20®, and Arachis oil.

Another embodiment comprises cyclosporin A, Volpo 131®, and Arachis oil.

Another embodiment comprises cyclosporin A, an Ethoxylated Fattv Ester or Oil, and Arachis oil.

Another embodiment comprises cyclosporin A, Acconon 6-C10®, and Arachis oil.

Another embodiment comprises cyclosporin A, Acconon CC-6®, and Arachis oil.

Another embodiment comprises cyclosporin A, Acconon CO-7®, and Arachis oil.

Another embodiment comprises cyclosporin A, Aldosperse 40/60 FG®, and Arachis oil.

Another embodiment comprises cyclosporin A, Aldosperse ML-23®, and Arachis oil.

Another embodiment comprises cyclosporin A, Aldosperse MS-20 FG®, and Arachis oil.

Another embodiment comprises cyclosporin A, Alkamuls EL-620®, and Arachis oil.

Another embodiment comprises cyclosporin A, Alkamuls EL-719®, and Arachis oil.

Another embodiment comprises cyclosporin A, Alkamuls EL-985®, and Arachis oil.

Another embodiment comprises cyclosporin A, Arlatone G®, and Arachis oil.

Another embodiment comprises cyclosporin A, Arlatone T®, and Arachis oil.

Another embodiment comprises cyclosporin A, Atlas G-1045A®, and Arachis oil.

Another embodiment comprises cyclosporin A, Atlas G-1086®, and Arachis oil.

Another embodiment comprises cyclosporin A, Atlas G-1087®, and Arachis oil.

Another embodiment comprises cyclosporin A, Atlas G-1089®, and Arachis oil.

Another embodiment comprises cyclosporin A, Atlas G-1096®, and Arachis oil.

Another embodiment comprises cyclosporin A, Atlas G-1292®, and Arachis oil.

Another embodiment comprises cyclosporin A, Atlas G-1293®, and Arachis oil.

Another embodiment comprises cyclosporin A, Atlas G-1300®, and Arachis oil.

Another embodiment comprises cyclosporin A, Atlas G-7076®, and Arachis oil.

Another embodiment comprises cyclosporin A, Capmul EMG®, and Arachis oil.

Another embodiment comprises cyclosporin A, Chemonic CO-40®, and Arachis oil.

Another embodiment comprises cyclosporin A, Chemonic LI3®, and Arachis oil.

Another embodiment comprises cyclosporin A, Chemonic LI-7®, and Arachis oil.

Another embodiment comprises cyclosporin A, Cirrasol GM®, and Arachis oil.

Another embodiment comprises cyclosporin A, Cremophor CO 40®, and Arachis oil.

Another embodiment comprises cyclosporin A, Cremophor CO 410®, and Arachis oil.

Another embodiment comprises cyclosporin A, Cremophor EL®, and Arachis oil.

Another embodiment comprises cyclosporin A, Cremophor GC7®, and Arachis oil.

Another embodiment comprises cyclosporin A, Cremophor RH-40®, and Arachis oil.

Another embodiment comprises cyclosporin A, Crovol A-40®, and Arachis oil.

Another embodiment comprises cyclosporin A, Crovol A-70®, and Arachis oil.

Another embodiment comprises cyclosporin A, Crovol M-70®, and Arachis oil.

Another embodiment comprises cyclosporin A, Crovol PK-70®, and Arachis oil.

Another embodiment comprises cyclosporin A, Cutina E-24®, and Arachis oil.

Another embodiment comprises cyclosporin A, Dacospin 12-R®, and Arachis oil.

Another embodiment comprises cyclosporin A, Dehymuls HRE-7®, and Arachis oil.

Another embodiment comprises cyclosporin A, DeSonic 30C®, and Arachis oil.

Another embodiment comprises cyclosporin A, DeSonic 36C®, and Arachis oil.

Another embodiment comprises cyclosporin A, DeSonic 40C®, and Arachis oil.

Another embodiment comprises cyclosporin A, Durfax 60®, and Arachis oil.

Another embodiment comprises cyclosporin A, Durfax 65®, and Arachis oil.

Another embodiment comprises cyclosporin A, Durfax 80®, and Arachis oil.

Another embodiment comprises cyclosporin A, Durfax EOM®, and Arachis oil.

Another embodiment comprises cyclosporin A, Eccoterge NF-2®, and Arachis oil.

Another embodiment comprises cyclosporin A, Emulpon CO-360®, and Arachis oil.

Another embodiment comprises cyclosporin A, Emulpon CO-550®, and Arachis oil.

Another embodiment comprises cyclosporin A, Emulsogen EL®, and Arachis oil.

Another embodiment comprises cyclosporin A, Emulsogen HCO 040®, and Arachis oil.

Another embodiment comprises cyclosporin A, Emulsogen HCO 060®, and Arachis oil.

Another embodiment comprises cyclosporin A, Emulsynt 1055®, and Arachis oil.

Another embodiment comprises cyclosporin A, Ethox 3095®, and Arachis oil.

Another embodiment comprises cyclosporin A, Eumulgin RO-40®, and Arachis oil.

Another embodiment comprises cyclosporin A, Genapol G-260®, and Arachis oil.

Another embodiment comprises cyclosporin A, Glycosperse L-20®, and Arachis oil.

Another embodiment comprises cyclosporin A, Glycosperse O-5®, and Arachis oil.

Another embodiment comprises cyclosporin A, Glycosperse O-20®, and Arachis oil.

Another embodiment comprises cyclosporin A, Glycosperse O-20 FG®, and Arachis oil.

Another embodiment comprises cyclosporin A, Glycosperse S-20®, and Arachis oil.

Another embodiment comprises cyclosporin A, Glycosperse S-20 FG®, and Arachis oil.

Another embodiment comprises cyclosporin A, Glycosperse TS-20®, and Arachis oil.

Another embodiment comprises cyclosporin A, Glycosperse TS-20 FG®, and Arachis oil.

Another embodiment comprises cyclosporin A, Hetan SL®, and Arachis oil.

Another embodiment comprises cyclosporin A, Hetan SO®, and Arachis oil.

Another embodiment comprises cyclosporin A, Hetan SS®, and Arachis oil.

Another embodiment comprises cyclosporin A, Hetoxide C-2®, and Arachis oil.

Another embodiment comprises cyclosporin A, Hetoxide C-9®, and Arachis oil.

Another embodiment comprises cyclosporin A, Hetoxide C-15®, and Arachis oil.

Another embodiment comprises cyclosporin A, Hetoxide C-25®, and Arachis oil.

Another embodiment comprises cyclosporin A, Hetoxide C-40®, and Arachis oil.

Another embodiment comprises cyclosporin A, Hetoxide C-200®, and Arachis oil.

Another embodiment comprises cyclosporin A, Hetoxide C-200-50%®, and Arachis oil.

Another embodiment comprises cyclosporin A, Hetoxide GC-30®, and Arachis oil.

Another embodiment comprises cyclosporin A, Hetoxide HC-60®, and Arachis oil.

Another embodiment comprises cyclosporin A, Ice No. 2®, and Arachis oil.

Another embodiment comprises cyclosporin A, Incrocas 30/40®, and Arachis oil.

Another embodiment comprises cyclosporin A, Lexol EC®, and Arachis oil.

Another embodiment comprises cyclosporin A, Lexol EO®, and Arachis oil.

Another embodiment comprises cyclosporin A, Lipocol HCO-40®, and Arachis oil.

Another embodiment comprises cyclosporin A, Lipocol HCO-60®, and Arachis oil.

Another embodiment comprises cyclosporin A, Lipocol O-3 Special®, and Arachis oil.

Another embodiment comprises cyclosporin A, Lipopeg 2-L®, and Arachis oil.

Another embodiment comprises cyclosporin A, Lipopeg 4-DO®, and Arachis oil.

Another embodiment comprises cyclosporin A, Lipopeg 4-DS®, and Arachis oil.

Another embodiment comprises cyclosporin A, Lipovol GTB®, and Arachis oil.

Another embodiment comprises cyclosporin A, Lonzest SML-20®, and Arachis oil.

Another embodiment comprises cyclosporin A, Lonzest SMO-20®, and Arachis oil.

Another embodiment comprises cyclosporin A, Lonzest SMS-20®, and Arachis oil.

Another embodiment comprises cyclosporin A, Lonzest STO-20®, and Arachis oil.

Another embodiment comprises cyclosporin A, Lonzest STS-20®, and Arachis oil.

Another embodiment comprises cyclosporin A, Lumulse GR-40®, and Arachis oil.

Another embodiment comprises cyclosporin A, Lumulse GRH-40®, and Arachis oil.

Another embodiment comprises cyclosporin A, Lumulse POE (7) GML®, and Arachis oil.

Another embodiment comprises cyclosporin A, Lumulse POE (12) Glyc®, and Arachis oil.

Another embodiment comprises cyclosporin A, Lumulse POE (40) MS KP®, and Arachis oil.

Another embodiment comprises cyclosporin A, Marlowet 4750®, and Arachis oil.

Another embodiment comprises cyclosporin A, Marlowet LVS®, and Arachis oil.

Another embodiment comprises cyclosporin A, Marlowet R 11®, and Arachis oil.

Another embodiment comprises cyclosporin A, Marlowet R 40®, and Arachis oil.

Another embodiment comprises cyclosporin A, Mazol 80 MGK®, and Arachis oil.

Another embodiment comprises cyclosporin A, Nonionic Emulsifier T-9®, and Arachis oil.

Another embodiment comprises cyclosporin A, Oronal LCG®, and Arachis oil.

Another embodiment comprises cyclosporin A, Polyderm PPI-CO-200®, and Arachis oil.

Another embodiment comprises cyclosporin A, Polyderm PPI-CO-40®, and Arachis oil.

Another embodiment comprises cyclosporin A, Rewoderm LI 520-70®, and Arachis oil.

Another embodiment comprises cyclosporin A, Ritapeg 150 DS®, and Arachis oil.

Another embodiment comprises cyclosporin A, Softigen 767®, and Arachis oil.

Another embodiment comprises cyclosporin A, Surfactol 318®, and Arachis oil.

Another embodiment comprises cyclosporin A, Surfactol 365®, and Arachis oil.

Another embodiment comprises cyclosporin A, Syn Lube 107®, and Arachis oil.

Another embodiment comprises cyclosporin A, Syn Lube 728®, and Arachis oil.

Another embodiment comprises cyclosporin A, Syn Lube 1632H®, and Arachis oil.

Another embodiment comprises cyclosporin A, Syn Lube 6277-A®, and Arachis oil.

Another embodiment comprises cyclosporin A, T-Det C-20®, and Arachis oil.

Another embodiment comprises cyclosporin A, T-Det C-40®, and Arachis oil.

Another embodiment comprises cyclosporin A, Tally 100 Plus®, and Arachis oil.

Another embodiment comprises cyclosporin A, Uniperol EL®, and Arachis oil.

Another embodiment comprises cyclosporin A, a Fatty Ester, and Arachis oil.

Another embodiment comprises cyclosporin A, Actralube-Syn 147®, and Arachis oil.

Another embodiment comprises cyclosporin A, Atlas G-1556®, and Arachis oil.

Another embodiment comprises cyclosporin A, Atlas G-1564®, and Arachis oil.

Another embodiment comprises cyclosporin A, Atlasol Base Oil S®, and Arachis oil.

Another embodiment comprises cyclosporin A, Base ML®, and Arachis oil.

Another embodiment comprises cyclosporin A, Base MT®, and Arachis oil.

Another embodiment comprises cyclosporin A, Cerasynt 303®, and Arachis oil.

Another embodiment comprises cyclosporin A, Dermol 1012®, and Arachis oil.

Another embodiment comprises cyclosporin A, Kemester 4000®, and Arachis oil.

Another embodiment comprises cyclosporin A, Lactipol S®, and Arachis oil.

Another embodiment comprises cyclosporin A, Magrabar PGO®, and Arachis oil.

Another embodiment comprises cyclosporin A, Mayco Base BFO®, and Arachis oil.

Another embodiment comprises cyclosporin A, Methyl Linoleate®, and Arachis oil.

Another embodiment comprises cyclosporin A, Pationic 122A®, and Arachis oil.

Another embodiment comprises cyclosporin A, Pationic 138C®, and Arachis oil.

Another embodiment comprises cyclosporin A, Pationic CSL®, and Arachis oil.

Another embodiment comprises cyclosporin A, Pationic ISL®, and Arachis oil.

Another embodiment comprises cyclosporin A, Pationic SBL®, and Arachis oil.

Another embodiment comprises cyclosporin A, Pationic SSL®, and Arachis oil.

Another embodiment comprises cyclosporin A, Ritasol®, and Arachis oil.

Another embodiment comprises cyclosporin A, Tego Alkanol CS 20®, and Arachis oil.

Another embodiment comprises cyclosporin A, Tego Alkanol L23 P®, and Arachis oil.

Another embodiment comprises cyclosporin A, Tego Alkanol S2®, and Arachis oil.

Another embodiment comprises cyclosporin A, Tego Alkanol S20 P®, and Arachis oil.

Another embodiment comprises cyclosporin A, Triemulsifier 600 MS®, and Arachis oil.

Another embodiment comprises cyclosporin A, a Glycerol Esters. and Arachis oil.

Another embodiment comprises cyclosporin A, Agro #9 Wint SBO®, and Arachis oil.

Another embodiment comprises cyclosporin A, Ahcovel Base 700®, and Arachis oil.

Another embodiment comprises cyclosporin A, Aldo HMS FG®, and Arachis oil.

Another embodiment comprises cyclosporin A, Aldo MLD®, and Arachis oil.

Another embodiment comprises cyclosporin A, Aldo MLD FG®, and Arachis oil.

Another embodiment comprises cyclosporin A, Aldo MO FG®, and Arachis oil.

Another embodiment comprises cyclosporin A, Aldo MS®, and Arachis oil.

Another embodiment comprises cyclosporin A, Aldo MS FG®, and Arachis oil.

Another embodiment comprises cyclosporin A, Aldo MS LG FG®, and Arachis oil.

Another embodiment comprises cyclosporin A, Aldo MSD®, and Arachis oil.

Another embodiment comprises cyclosporin A, Aldo MSD FG®, and Arachis oil.

Another embodiment comprises cyclosporin A, Aldosperse O-20 FG®, and Arachis oil.

Another embodiment comprises cyclosporin A, Aldosperse TS-20 FG®, and Arachis oil.

Another embodiment comprises cyclosporin A, Aldosperse TS-40 F®, and Arachis oil.

Another embodiment comprises cyclosporin A, Arlacel 165®, and Arachis oil.

Another embodiment comprises cyclosporin A, Arlacel 186®, and Arachis oil.

Another embodiment comprises cyclosporin A, Capmul GMO®, and Arachis oil.

Another embodiment comprises cyclosporin A, Capmul GMS®, and Arachis oil.

Another embodiment comprises cyclosporin A, Caprol 3GO®, and Arachis oil.

Another embodiment comprises cyclosporin A, Caprol 3GVS®, and Arachis oil.

Another embodiment comprises cyclosporin A, Caprol 6G2S®, and Arachis oil.

Another embodiment comprises cyclosporin A, Caprol 10G40®, and Arachis oil.

Another embodiment comprises cyclosporin A, Caprol 10G100®, and Arachis oil.

Another embodiment comprises cyclosporin A, Caprol ET®, and Arachis oil.

Another embodiment comprises cyclosporin A, Caprol PGE860®, and Arachis oil.

Another embodiment comprises cyclosporin A, Cerasynt 945®, and Arachis oil.

Another embodiment comprises cyclosporin A, Cerasynt GMS®, and Arachis oil.

Another embodiment comprises cyclosporin A, Cerasynt Q®, and Arachis oil.

Another embodiment comprises cyclosporin A, Cerasynt SD®, and Arachis oil.

Another embodiment comprises cyclosporin A, Cerasynt WM®, and Arachis oil.

Another embodiment comprises cyclosporin A, Chemsperse 14®, and Arachis oil.

Another embodiment comprises cyclosporin A, Cremophor GO-32®, and Arachis oil.

Another embodiment comprises cyclosporin A, Cremophor GS11®, and Arachis oil.

Another embodiment comprises cyclosporin A, Cremophor GS-32®, and Arachis oil.

Another embodiment comprises cyclosporin A, Cutina KD-16®, and Arachis oil.

Another embodiment comprises cyclosporin A, Dehymuls PGPH®, and Arachis oil.

Another embodiment comprises cyclosporin A, Dermol DGDIS®, and Arachis oil.

Another embodiment comprises cyclosporin A, Dermol DGMIS®, and Arachis oil.

Another embodiment comprises cyclosporin A, Dermol G-76®, and Arachis oil.

Another embodiment comprises cyclosporin A, Dermol G-7DI®, and Arachis oil.

Another embodiment comprises cyclosporin A, Dermol NGDI®, and Arachis oil.

Another embodiment comprises cyclosporin A, Dermolan GLH®, and Arachis oil.

Another embodiment comprises cyclosporin A, Drewmulse GMO®, and Arachis oil.

Another embodiment comprises cyclosporin A, Drewpol 3-5-M®, and Arachis oil.

Another embodiment comprises cyclosporin A, Durlac 100 W®, and Arachis oil.

Another embodiment comprises cyclosporin A, Dur-Lo®, and Arachis oil.

Another embodiment comprises cyclosporin A, Dynasan 118®, and Arachis oil.

Another embodiment comprises cyclosporin A, EC-25®, and Arachis oil.

Another embodiment comprises cyclosporin A, EM 40®, and Arachis oil.

Another embodiment comprises cyclosporin A, Emerest 2400®, and Arachis oil.

Another embodiment comprises cyclosporin A, Emerest 2452®, and Arachis oil.

Another embodiment comprises cyclosporin A, Empilan G-26®, and Arachis oil.

Another embodiment comprises cyclosporin A, Genapol TSM®, and Arachis oil.

Another embodiment comprises cyclosporin A, Hostacerin DGI®, and Arachis oil.

Another embodiment comprises cyclosporin A, Hostacerin DGL®, and Arachis oil.

Another embodiment comprises cyclosporin A, Hostacerin DGMS®, and Arachis oil.

Another embodiment comprises cyclosporin A, Hostacerin DGSB®, and Arachis oil.

Another embodiment comprises cyclosporin A, Ice No. 2®, and Arachis oil.

Another embodiment comprises cyclosporin A, Imwitor 742®, and Arachis oil.

Another embodiment comprises cyclosporin A, Imwitor 780 K®, and Arachis oil.

Another embodiment comprises cyclosporin A, Imwitor 960 Flakes®, and Arachis oil.

Another embodiment comprises cyclosporin A, isolan GI 34®, and Arachis oil.

Another embodiment comprises cyclosporin A, Isolan GO 33®, and Arachis oil.

Another embodiment comprises cyclosporin A, Kemester 1000®, and Arachis oil.

Another embodiment comprises cyclosporin A, Kemester 2000®, and Arachis oil.

Another embodiment comprises cyclosporin A, Kemester 2000®, and Arachis oil.

Another embodiment comprises cyclosporin A, Kemester 6000SE®, and Arachis oil.

Another embodiment comprises cyclosporin A, Lamecreme DGE 18®, and Arachis oil.

Another embodiment comprises cyclosporin A, Lexemul 515®, and Arachis oil.

Another embodiment comprises cyclosporin A, Lexemul 561®, and Arachis oil.

Another embodiment comprises cyclosporin A, Lexemul AR®, and Arachis oil.

Another embodiment comprises cyclosporin A, Lexemul AS®, and Arachis oil.

Another embodiment comprises cyclosporin A, Lexemul GDL®, and Arachis oil.

Another embodiment comprises cyclosporin A, Lexemul T®, and Arachis oil.

Another embodiment comprises cyclosporin A, Lipomulse 165®, and Arachis oil.

Another embodiment comprises cyclosporin A, Lumulse GML K®, and Arachis oil.

Another embodiment comprises cyclosporin A, Lumulse GMO K®, and Arachis oil.

Another embodiment comprises cyclosporin A, Lumulse GMR K®, and Arachis oil.

Another embodiment comprises cyclosporin A, Lumulse GMT K®, and Arachis oil.

Another embodiment comprises cyclosporin A, Magrabar GMC®, and Arachis oil.

Another embodiment comprises cyclosporin A, Magrabar GMO-CK®, and Arachis oil.

Another embodiment comprises cyclosporin A, Magrabar MDG-5050®, and Arachis oil.

Another embodiment comprises cyclosporin A, Magrabar PGO-315®, and Arachis oil.

Another embodiment comprises cyclosporin A, Magrabar PGO-1010®, and Arachis oil.

Another embodiment comprises cyclosporin A, Mazol 300K®, and Arachis oil.

Another embodiment comprises cyclosporin A, Mazol GMO-K®, and Arachis oil.

Another embodiment comprises cyclosporin A, Mazol GMS-K®, and Arachis oil.

Another embodiment comprises cyclosporin A, Mazol PG031-K®, and Arachis oil.

Another embodiment comprises cyclosporin A, Miglyol 812®, and Arachis oil.

Another embodiment comprises cyclosporin A, Norfox 165C®, and Arachis oil.

Another embodiment comprises cyclosporin A, Schercemol GMIS®, and Arachis oil.

Another embodiment comprises cyclosporin A, Tegin®, and Arachis oil.

Another embodiment comprises cyclosporin A, Tegin 4100 Pellets®, and Arachis oil.

Another embodiment comprises cyclosporin A, Tegin M Pellets®, and Arachis oil.

Another embodiment comprises cyclosporin A, Tegin OV®, and Arachis oil.

Another embodiment comprises cyclosporin A, Teginacid H®, and Arachis oil.

Another embodiment comprises cyclosporin A, Tego Cosmo P813®, and Arachis oil.

Another embodiment comprises cyclosporin A, Wickenol 535®, and Arachis oil.

Another ermbodiment comprises cyclosporin A, Witconol 14®, and Arachis oil.

Another embodiment comprises cyclosporin A, Witconol 14®, and Arachis oil.

Another embodiment comprises cyclosporin A, Witconol 14F®, and Arachis oil.

Another embodiment comprises cyclosporin A, Witconol 18L®, and Arachis oil.

Another embodiment comprises cyclosporin A, Witconol GOT®, and Arachis oil.

Another embodiment comprises cyclosporin A, Witconol MST®, and Arachis oil.

Another embodiment comprises cyclosporin A, Witconol RHT®, and Arachis oil.

Another embodiment comprises cyclosporin A, a Glycol Ester, and Arachis oil.

Another embodiment comprises cyclosporin A, Alkamuls 600 DO®, and Arachis oil.

Another embodiment comprises cyclosporin A, Alkamuls SEG®, and Arachis oil.

Another embodiment comprises cyclosporin A, Atlas EM-2®, and Arachis oil.

Another embodiment comprises cyclosporin A, Cerasynt IP®, and Arachis oil.

Another embodiment comprises cyclosporin A, Cerasynt M®, and Arachis oil.

Another embodiment comprises cyclosporin A, Cerasynt MN®, and Arachis oil.

Another embodiment comprises cyclosporin A, Cerasynt PA®, and Arachis oil.

Another embodiment comprises cyclosporin A, Chemsperse EGDS®, and Arachis oil.

Another embodiment comprises cyclosporin A, Chemsperse EGMS®, and Arachis oil.

Another embodiment comprises cyclosporin A, Colonial Monolaurin®, and Arachis oil.

Another embodiment comprises cyclosporin A, DeMuls SGE-95®, and Arachis oil.

Another embodiment comprises cyclosporin A, Eccoterge 200®, and Arachis oil.

Another embodiment comprises cyclosporin A, Emerest 2380®, and Arachis oil.

Another embodiment comprises cyclosporin A, Ethox 2610®, and Arachis oil.

Another embodiment comprises cyclosporin A, Ethox DO-9®, and Arachis oil.

Another embodiment comprises cyclosporin A, Ethox DO-14®, and Arachis oil.

Another embodiment comprises cyclosporin A, Ethox SO-9®, and Arachis oil.

Another embodiment comprises cyclosporin A, Fizul MD-318®, and Arachis oil.

Another embodiment comprises cyclosporin A, Genapol EGDS-VHP®, and Arachis oil.

Another embodiment comprises cyclosporin A, Genapol TS Powder®, and Arachis oil.

Another embodiment comprises cyclosporin A, Hostacerin WO®, and Arachis oil.

Another embodiment comprises cyclosporin A, Inversol 140®, and Arachis oil.

Another embodiment comprises cyclosporin A, Kemester 104®, and Arachis oil.

Another embodiment comprises cyclosporin A, Kemester 205®, and Arachis oil.

Another embodiment comprises cyclosporin A, Kemester 226®, and Arachis oil.

Another embodiment comprises cyclosporin A, Kemester 5221SE®, and Arachis oil.

Another embodiment comprises cyclosporin A, Kemester EGDS®, and Arachis oil.

Another embodiment comprises cyclosporin A, Lexemul EGDS®, and Arachis oil.

Another embodiment comprises cyclosporin A, Lexemul EGMS®, and Arachis oil.

Another embodiment comprises cyclosporin A, Lexemul P®, and Arachis oil.

Another embodiment comprises cyclosporin A, Lipo DGLS®, and Arachis oil.

Another embodiment comprises cyclosporin A, Lipo EGDS®, and Arachis oil.

Another embodiment comprises cyclosporin A, Lipo PGMS®, and Arachis oil.

Another embodiment comprises cyclosporin A, Liposorb S-4®, and Arachis oil.

Another embodiment comprises cyclosporin A, Liposorb TO-20®, and Arachis oil.

Another embodiment comprises cyclosporin A, Lumulse PGO®, and Arachis oil.

Another embodiment comprises cyclosporin A, Mackester EGDS®, and Arachis oil.

Another embodiment comprises cyclosporin A, Mackester EGMS®, and Arachis oil.

Another embodiment comprises cyclosporin A, Mackester GSTP®, and Arachis oil.

Another embodiment comprises cyclosporin A, Mackester Series®, and Arachis oil.

Another embodiment comprises cyclosporin A, Magrabar PDG-50®, and Arachis oil.

Another embodiment comprises cyclosporin A, Mapeg 6000 DS®, and Arachis oil.

Another embodiment comprises cyclosporin A, Marlowet 4702®, and Arachis oil.

Another embodiment comprises cyclosporin A, Monalube 305®, and Arachis oil.

Another embodiment comprises cyclosporin A, Monalube 310®, and Arachis oil.

Another embodiment comprises cyclosporin A, Monalube 315®, and Arachis oil.

Another embodiment comprises cyclosporin A, Monalube 320®, and Arachis oil.

Another embodiment comprises cyclosporin A, Monalube 325®, and Arachis oil.

Another embodiment comprises cyclosporin A, Monalube 330®, and Arachis oil.

Another embodiment comprises cyclosporin A, Naturechem PGHS®, and Arachis oil.

Another embodiment comprises cyclosporin A, Polycastorol PLO-840®, and Arachis oil.

Another embodiment comprises cyclosporin A, Polytex 10M®, and Arachis oil.

Another embodiment comprises cyclosporin A, Ritasynt IP®, and Arachis oil.

Another embodiment comprises cyclosporin A, Ross Chem PEG 600 DT®, and Arachis oil.

Another embodiment comprises cyclosporin A, Schercemol PGMS®, and Arachis oil.

Another embodiment comprises cyclosporin A, Sponto H-44C®, and Arachis oil.

Another embodiment comprises cyclosporin A, Tegin G®, and Arachis oil.

Another embodiment comprises cyclosporin A, Witbreak DGE-182®, and Arachis oil.

Another embodiment comprises cyclosporin A, Witbreak DGE-182®, and Arachis oil.

Another embodiment comprises cyclosporin A, Witbreak DRA-21®, and Arachis oil.

Another embodiment comprises cyclosporin A, Witbreak DRA-21®, and Arachis oil.

Another embodiment comprises cyclosporin A, Witbreak DRA-50®, and Arachis oil.

Another embodiment comprises cyclosporin A, Witbreak DRA-50®, and Arachis oil.

Another embodiment comprises cyclosporin A, Witconol F26-46®, and Arachis oil.

Another embodiment comprises cyclosporin A, Witconol H-32®, and Arachis oil.

Another embodiment comprises cyclosporin A, Witconol H-33®, and Arachis oil.

Another embodiment comprises cyclosporin A, Witconol H-35A®, and Arachis oil.

Another embodiment comprises cyclosporin A, Witconol RHP®, and Arachis oil.

Another embodiment comprises cyclosporin A, a Lanolin-based Derivative and Arachis oil.

Another embodiment comprises cyclosporin A, Amerchol CAB®, and Arachis oil.

Another embodiment comprises cyclosporin A, Amerchol L-101®, and Arachis oil.

Another embodiment comprises cyclosporin A, Amerlate LFA-LO®, and Arachis oil.

Another embodiment comprises cyclosporin A, Ameriate P®, and Arachis oil.

Another embodiment comprises cyclosporin A, Barre Common Degras®, and Arachis oil.

Another embodiment comprises cyclosporin A, Cholesterol NF®, and Arachis oil.

Another embodiment comprises cyclosporin A, Crodalan AWS®, and Arachis oil.

Another embodiment comprises cyclosporin A, Crodalan LA®, and Arachis oil.

Another embodiment comprises cyclosporin A, Emery 1650®, and Arachis oil.

Another embodiment comprises cyclosporin A, Emery 1650®, and Arachis oil.

Another embodiment comprises cyclosporin A, Emery 1740®, and Arachis oil.

Another embodiment comprises cyclosporin A, Emery 1740®, and Arachis oil.

Another embodiment comprises cyclosporin A, Forlan 500®, and Arachis oil.

Another embodiment comprises cyclosporin A, Forlan L®, and Arachis oil.

Another embodiment comprises cyclosporin A, Laneto 50®, and Arachis oil.

Another embodiment comprises cyclosporin A, Laneto 100®, and Arachis oil.

Another embodiment comprises cyclosporin A, Laneto AWS®, and Arachis oil.

Another embodiment comprises cyclosporin A, Lanfrax 1776®, and Arachis oil.

Another embodiment comprises cyclosporin A, Lanfrax 1776®, and Arachis oil.

Another embodiment comprises cyclosporin A, Lanogel 21®, and Arachis oil.

Another embodiment comprises cyclosporin A, Lipolan®, and Arachis oil.

Another embodiment comprises cyclosporin A, Lipolan 31®, and Arachis oil.

Another embodiment comprises cyclosporin A, OHlan®, and Arachis oil.

Another embodiment comprises cyclosporin A, Polychol 5®, and Arachis oil.

Another embodiment comprises cyclosporin A, Polychol 15®, and Arachis oil.

Another embodiment comprises cyclosporin A, Ritacetyl®, and Arachis oil.

Another embodiment comprises cyclosporin A, Ritachol®, and Arachis oil.

Another embodiment comprises cyclosporin A, Ritahydrox®, and Arachis oil.

Another embodiment comprises cyclosporin A, Ritalafa®, and Arachis oil.

Another embodiment comprises cyclosporin A, Ritalan®, and Arachis oil.

Another embodiment comprises cyclosporin A, Ritalan AWS®, and Arachis oil.

Another embodiment comprises cyclosporin A, Ritalan C®, and Arachis oil.

Another embodiment comprises cyclosporin A, Ritawax®, and Arachis oil.

Another embodiment comprises cyclosporin A, Ritawax AEO®, and Arachis oil.

Another embodiment comprises cyclosporin A, Ritawax ALA®, and Arachis oil.

Another embodiment comprises cyclosporin A, Solan/Solan 50/Super Solan®, and Arachis oil.

Another embodiment comprises cyclosporin A, Super Hartolan/Hartolan®, and Arachis oil.

Another embodiment comprises cyclosporin A, Supersat AWS-4®, and Arachis oil.

Another embodiment comprises cyclosporin A, Supersat AWS-24®, and Arachis oil.

Another embodiment comprises cyclosporin A, a Lecithin or Lecithin Derivative, and Arachis oil.

Another embodiment comprises cyclosporin A, Alcolec®, and Arachis oil.

Another embodiment comprises cyclosporin A, Lecithin®, and Arachis oil.

Another embodiment comprises cyclosporin A, Lexin K®, and Arachis oil.

Another embodiment comprises cyclosporin A, Natipide®, and Arachis oil.

Another embodiment comprises cyclosporin A, a Lignin or Lignin Derivative, and Arachis oil.

Another embodiment comprises cyclosporin A, Diwatex XP 9®, and Arachis oil.

Another embodiment comprises cyclosporin A, Dynasperse LCD®, and Arachis oil.

Another embodiment comprises cyclosporin A, Indulin SAL®, and Arachis oil.

Another embodiment comprises cyclosporin A, Indulin W-1®, and Arachis oil.

Another embodiment comprises cyclosporin A, Indulin W-5®, and Arachis oil.

Another embodiment comprises cyclosporin A, Lignosol FTA®, and Arachis oil.

Another embodiment comprises cyclosporin A, Lignosol SFX-65®, and Arachis oil.

Another embodiment comprises cyclosporin A, Marasperse 52 CP®, and Arachis oil.

Another embodiment comprises cyclosporin A, Marasperse AG®, and Arachis oil.

Another embodiment comprises cyclosporin A, Marasperse CBOS-4®, and Arachis oil.

Another embodiment comprises cyclosporin A, Ufoxane 2®, and Arachis oil.

Another embodiment comprises cyclosporin A, a Methyl Ester, and Arachis oil.

Another embodiment comprises cyclosporin A, E.B. Cleaner AK®, and Arachis oil.

Another embodiment comprises cyclosporin A, Oleocal ME-70®, and Arachis oil.

Another embodiment comprises cyclosporin A, Trade Name Company®, and Arachis oil.

Another embodiment comprises cyclosporin A, Methyl Esters (cont'd)®, and Arachis oil.

Another embodiment comprises cyclosporin A, Oleocal ME-92®, and Arachis oil.

Another embodiment comprises cyclosporin A, Oleocal ME-112®, and Arachis oil.

Another embodiment comprises cyclosporin A, Oleocal ME-130®, and Arachis oil.

Another embodiment comprises cyclosporin A, a Monoglyceride or a Derivative thereof, and Arachis oil.

Another embodiment comprises cyclosporin A, Dynacet 211®, and Arachis oil.

Another embodiment comprises cyclosporin A, Hetsorb S-20®, and Arachis oil.

Another embodiment comprises cyclosporin A, lmwitor 191®, and Arachis oil.

Another embodiment comprises cyclosporin A, lmwitor 370®, and Arachis oil.

Another embodiment comprises cyclosporin A, Imwitor 375®, and Arachis oil.

Another embodiment comprises cyclosporin A, lmwitor 900®, and Arachis oil.

Another embodiment comprises cyclosporin A, lmwitor 945®, and Arachis oil.

Another embodiment comprises cyclosporin A, lmwitor 2020®, and Arachis oil.

Another embodiment comprises cyclosporin A, Kemester 5500®, and Arachis oil.

Another embodiment comprises cyclosporin A, Kemester 6000®, and Arachis oil.

Another embodiment comprises cyclosporin A, Magrabar GMC®, and Arachis oil.

Another embodiment comprises cyclosporin A, Magrabar GMO-CK®, and Arachis oil.

Another embodiment comprises cyclosporin A, Magrabar GPC-10®, and Arachis oil.

Another embodiment comprises cyclosporin A, Magrabar MDG-5050®, and Arachis oil.

Another embodiment comprises cyclosporin A, Monalube 335®, and Arachis oil.

Another embodiment comprises cyclosporin A, Monoglycerides Glyceryl Monestearate Archer Daniels Midland Company®, and Arachis oil.

Another embodiment comprises cyclosporin A, Rita GMS®, and Arachis oil.

Another embodiment comprises cyclosporin A, Ritamulse SCG®, and Arachis oil.

Another embodiment comprises cyclosporin A, Softigen 701®, and Arachis oil.

Another embodiment comprises cyclosporin A, Tally 100 Plus®, and Arachis oil.

Another embodiment comprises cyclosporin A, a Polyethylene Glycol, and Arachis oil.

Another embodiment comprises cyclosporin A, Emulgade PL 68/50®, and Arachis oil.

Another embodiment comprises cyclosporin A, Lumulse PEG®, and Arachis oil.

Another embodiment comprises cyclosporin A, Rhodasurl PEG-400®, and Arachis oil.

Another embodiment comprises cyclosporin A, Rhodasurf PEG-600®, and Arachis oil.

Another embodiment comprises cyclosporin A, Witconol PEG-400®, and Arachis oil.

Another embodiment comprises cyclosporin A, a Polymeric Surfactant, and Arachis oil.

Another embodiment comprises cyclosporin A, Acritamer PNC-EG®, and Arachis oil.

Another embodiment comprises cyclosporin A, Ag-Rho DEP-775®, and Arachis oil.

Another embodiment comprises cyclosporin A, APG 325N Glycoside®, and Arachis oil.

Another embodiment comprises cyclosporin A, Aristoflex AVC®, and Arachis oil.

Another embodiment comprises cyclosporin A, Aristoflex HMB®, and Arachis oil.

Another embodiment comprises cyclosporin A, Burco NPS-225®, and Arachis oil.

Another embodiment comprises cyclosporin A, Burco NPS-816®, and Arachis oil.

Another embodiment comprises cyclosporin A, Chemccinate 5603®, and Arachis oil.

Another embodiment comprises cyclosporin A, Cosmedia Guar C-261 N®, and Arachis oil.

Another embodiment comprises cyclosporin A, Gantrez S-95®, and Arachis oil.

Another embodiment comprises cyclosporin A, Glucopon 220 UP®, and Arachis oil.

Another embodiment comprises cyclosporin A, Glucopon 225 DK®, and Arachis oil.

Another embodiment comprises cyclosporin A, Glucopon 425 N®, and Arachis oil.

Another embodiment comprises cyclosporin A, Glucopon 600 UP®, and Arachis oil.

Another embodiment comprises cyclosporin A, Glucopon 625 UP®, and Arachis oil.

Another embodiment comprises cyclosporin A, Pemulen 1621®, and Arachis oil.

Another embodiment comprises cyclosporin A, Pemulen 1622®, and Arachis oil.

Another embodiment comprises cyclosporin A, Pemulen TR-1®, and Arachis oil.

Another embodiment comprises cyclosporin A, Pemulen TR-2®, and Arachis oil.

Another embodiment comprises cyclosporin A, Plantacare 818®, and Arachis oil.

Another embodiment comprises cyclosporin A, Plantapon LGC Sorb®, and Arachis oil.

Another embodiment comprises cyclosporin A, Plantaren 1200N®, and Arachis oil.

Another embodiment comprises cyclosporin A, Plantaren 2000N®, and Arachis oil.

Another embodiment comprises cyclosporin A, Viscolam AT 64®, and Arachis oil.

Another embodiment comprises cyclosporin A, Viscolam AT 64P®, and Arachis oil.

Another embodiment comprises cyclosporin A, Viscolam AT 100®, and Arachis oil.

Another embodiment comprises cyclosporin A, Viscolam MAC 7®, and Arachis oil.

Another embodiment comprises cyclosporin A, Viscolam SMC 20®, and Arachis oil.

Another embodiment comprises cyclosporin A, Witbreak RTC-323®, and Arachis oil.

Another embodiment comprises cyclosporin A, WSI 3700®, and Arachis oil.

Another embodiment comprises cyclosporin A, a Propoxylated or Ethoxylated Fatty Acid, Alcohol, or Alkyl Phenol, and Arachis oil.

Another embodiment comprises cyclosporin A, Antarox AA-60®, and Arachis oil.

Another embodiment comprises cyclosporin A, Antarox LF-224®, and Arachis oil.

Another embodiment comprises cyclosporin A, Burcomul DFE-45®, and Arachis oil.

Another embodiment comprises cyclosporin A, Burcoterge LFE-1000®, and Arachis oil.

Another embodiment comprises cyclosporin A, Chemal LF-25B®, and Arachis oil.

Another embodiment comprises cyclosporin A, Chemal LF-40B®, and Arachis oil.

Another embodiment comprises cyclosporin A, Dehypon LS-36®, and Arachis oil.

Another embodiment comprises cyclosporin A, Dehypon LS-36®, and Arachis oil.

Another embodiment comprises cyclosporin A, Dehypon LS-54®, and Arachis oil.

Another embodiment comprises cyclosporin A, Dehypon LS-54®, and Arachis oil.

Another embodiment comprises cyclosporin A, Delonic 100 VLF®, and Arachis oil.

Another embodiment comprises cyclosporin A, Delonic LF-60 MOD®, and Arachis oil.

Another embodiment comprises cyclosporin A, Empiderm B®, and Arachis oil.

Another embodiment comprises cyclosporin A, Ethylan 1206®, and Arachis oil.

Another embodiment comprises cyclosporin A, Ethylan NS-500K®, and Arachis oil.

Another embodiment comprises cyclosporin A, Ethylan NS-500LQ®, and Arachis oil.

Another embodiment comprises cyclosporin A, Genapol 1392®, and Arachis oil.

Another embodiment comprises cyclosporin A, Genapol 2317®, and Arachis oil.

Another embodiment comprises cyclosporin A, Genapol 26EP710®, and Arachis oil.

Another embodiment comprises cyclosporin A, Genapol EP 1022®, and Arachis oil.

Another embodiment comprises cyclosporin A, Genapol EP 1024®, and Arachis oil.

Another embodiment comprises cyclosporin A, Genapol EP 6068®, and Arachis oil.

Another embodiment comprises cyclosporin A, Genapol NP915®, and Arachis oil.

Another embodiment comprises cyclosporin A, Kieralon MFB®, and Arachis oil.

Another embodiment comprises cyclosporin A, Lumisolve CSA-80 V®, and Arachis oil.

Another embodiment comprises cyclosporin A, Marlowet 5001®, and Arachis oil.

Another embodiment comprises cyclosporin A, Marlox FK 64®, and Arachis oil.

Another embodiment comprises cyclosporin A, Marlox MO 124®, and Arachis oil.

Another embodiment comprises cyclosporin A, Marlox S 58®, and Arachis oil.

Another embodiment comprises cyclosporin A, Nonatell 1003®, and Arachis oil.

Another embodiment comprises cyclosporin A, Nonatell 1038®, and Arachis oil.

Another embodiment comprises cyclosporin A, Nonatell 1052®, and Arachis oil.

Another embodiment comprises cyclosporin A, Nonatell 1061®, and Arachis oil.

Another embodiment comprises cyclosporin A, Nonatell 1075®, and Arachis oil.

Another embodiment comprises cyclosporin A, Nonatell 1088®, and Arachis oil.

Another embodiment comprises cyclosporin A, Nonatell 1123®, and Arachis oil.

Another embodiment comprises cyclosporin A, Nonatell 1153®, and Arachis oil.

Another embodiment comprises cyclosporin A, Nonatell 1161®, and Arachis oil.

Another embodiment comprises cyclosporin A, Nonatell 1172®, and Arachis oil.

Another embodiment comprises cyclosporin A, Nonatell 1181®, and Arachis oil.

Another embodiment comprises cyclosporin A, Nonox 36®, and Arachis oil.

Another embodiment comprises cyclosporin A, Procetyl AWS®, and Arachis oil.

Another embodiment comprises cyclosporin A, Sandoxylate SX 412®, and Arachis oil.

Another embodiment comprises cyclosporin A, Sandoxylate SX 418®, and Arachis oil.

Another embodiment comprises cyclosporin A, Surfonic JL-80X®, and Arachis oil.

Another embodiment comprises cyclosporin A, Surfonic JL-80X-B1®, and Arachis oil.

Another embodiment comprises cyclosporin A, Surfonic L4-29X®, and Arachis oil.

Another embodiment comprises cyclosporin A, Surfonic LF®, and Arachis oil.

Another embodiment comprises cyclosporin A, T-Det A826®, and Arachis oil.

Another embodiment comprises cyclosporin A, T-Det LF-416®, and Arachis oil.

Another embodiment comprises cyclosporin A, Tergitol Min-Foam 1X®, and Arachis oil.

Another embodiment comprises cyclosporin A, Tergitol Min-Foam 2X®, and Arachis oil.

Another embodiment comprises cyclosporin A, Triton CF-21®, and Arachis oil.

Another embodiment comprises cyclosporin A, Triton CF-76®, and Arachis oil.

Another embodiment comprises cyclosporin A, Triton XL-80N®, and Arachis oil.

Another embodiment comprises cyclosporin A, Witconol NS-98®, and Arachis oil.

Another embodiment comprises cyclosporin A, Witconol NS-108LQ®, and Arachis oil.

Another embodiment comprises cyclosporin A, Witconol NS-145®, and Arachis oil.

Another embodiment comprises cyclosporin A, Witconol NS-179®, and Arachis oil.

Another embodiment comprises cyclosporin A, a Protein-based Surfactant, and Arachis oil.

Another embodiment comprises cyclosporin A, AminoFoam W®, and Arachis oil.

Another embodiment comprises cyclosporin A, Amiter LGOD-2®, and Arachis oil.

Another embodiment comprises cyclosporin A, Amiter LGS-2®, and Arachis oil.

Another embodiment comprises cyclosporin A, Amiter LGS-5®, and Arachis oil.

Another embodiment comprises cyclosporin A, Lamepon S®, and Arachis oil.

Another embodiment comprises cyclosporin A, Lamepon S®, and Arachis oil.

Another embodiment comprises cyclosporin A, Maypon 4C®, and Arachis oil.

Another embodiment comprises cyclosporin A, May-Tein C®, and Arachis oil.

Another embodiment comprises cyclosporin A, May-Tein CT®, and Arachis oil.

Another embodiment comprises cyclosporin A, May-Tein KTS®, and Arachis oil.

Another embodiment comprises cyclosporin A, May-Tein SY®, and Arachis oil.

Another embodiment comprises cyclosporin A, Plantapon S®, and Arachis oil.

Another embodiment comprises cyclosporin A, Proteol APL®, and Arachis oil.

Another embodiment comprises cyclosporin A, Proteol OAT®, and Arachis oil.

Another embodiment comprises cyclosporin A, Pyroter CPI-40®, and Arachis oil.

Another embodiment comprises cyclosporin A, Pyroter GPI-25®, and Arachis oil.

Another embodiment comprises cyclosporin A, Supro-Tein S®, and Arachis oil.

Another embodiment comprises cyclosporin A, Supro-Tein V®, and Arachis oil.

Another embodiment comprises cyclosporin A,® and Arachis oil.

Another embodiment comprises cyclosporin A, a Sarcosine Derivatives, and Arachis oil.

Another embodiment comprises cyclosporin A, Crodasinic LS-30®, and Arachis oil.

Another embodiment comprises cyclosporin A, Vanseal CS®, and Arachis oil.

Another embodiment comprises cyclosporin A, Vanseal LS®, and Arachis oil.

Another embodiment comprises cyclosporin A, Vanseal MS®, and Arachis oil.

Another embodiment comprises cyclosporin A, Vanseal NACS-30®, and Arachis oil.

Another embodiment comprises cyclosporin A, Vanseal NALS-95®, and Arachis oil.

Another embodiment comprises cyclosporin A, Vanseal OS®, and Arachis oil.

Another embodiment comprises cyclosporin A,® and Arachis oil.

Another embodiment comprises cyclosporin A, a Silicone-based Surfactante, and Arachis oil.

Another embodiment comprises cyclosporin A, Abil-B-9950®, and Arachis oil.

Another embodiment comprises cyclosporin A, Abil Care 85®, and Arachis oil.

Another embodiment comprises cyclosporin A, Abil EM 90®, and Arachis oil.

Another embodiment comprises cyclosporin A, Abil EM 97®, and Arachis oil.

Another embodiment comprises cyclosporin A, Abil WE-09®, and Arachis oil.

Another embodiment comprises cyclosporin A, Dow Corning 1248 Fluid®, and Arachis oil.

Another embodiment comprises cyclosporin A, Dow Corning 3225C®, and Arachis oil.

Another embodiment comprises cyclosporin A, Dow Corning 5200®, and Arachis oil.

Another embodiment comprises cyclosporin A, Dow Corning Q4-3667®, and Arachis oil.

Another embodiment comprises cyclosporin A, Monasil PCA®, and Arachis oil.

Another embodiment comprises cyclosporin A, Monasil PDM®, and Arachis oil.

Another embodiment comprises cyclosporin A, Monasil PLN®, and Arachis oil.

Another embodiment comprises cyclosporin A, Polyderm PPI-SI-WS®, and Arachis oil.

Another embodiment comprises cyclosporin A, Troysol 380W®, and Arachis oil.

Another embodiment comprises cyclosporin A, Troysol S366®, and Arachis oil.

Another embodiment comprises cyclosporin A, a Sorbitan Derivative, and Arachis oil.

Another embodiment comprises cyclosporin A, Alkamuls SML®, and Arachis oil.

Another embodiment comprises cyclosporin A, Alkamuls SMO®, and Arachis oil.

Another embodiment comprises cyclosporin A, Alkamuls STO®, and Arachis oil.

Another embodiment comprises cyclosporin A, Arlacel 20®, and Arachis oil.

Another embodiment comprises cyclosporin A, Arlacel 40®, and Arachis oil.

Another embodiment comprises cyclosporin A, Arlacel 60®, and Arachis oil.

Another embodiment comprises cyclosporin A, Arlacel 80®, and Arachis oil.

Another embodiment comprises cyclosporin A, Arlacel C®, and Arachis oil.

Another embodiment comprises cyclosporin A, Armul 21®, and Arachis oil.

Another embodiment comprises cyclosporin A, Atlox 80®, and Arachis oil.

Another embodiment comprises cyclosporin A, Atlox 847®, and Arachis oil.

Another embodiment comprises cyclosporin A, Atlox 1045A®, and Arachis oil.

Another embodiment comprises cyclosporin A, Canarcel 20®, and Arachis oil.

Another embodiment comprises cyclosporin A, Canarcel 60®, and Arachis oil.

Another embodiment comprises cyclosporin A, Canarcel 80®, and Arachis oil.

Another embodiment comprises cyclosporin A, Canarcel TW 20®, and Arachis oil.

Another embodiment comprises cyclosporin A, Canarcel TW 60®, and Arachis oil.

Another embodiment comprises cyclosporin A, Canarcel TW 80®, and Arachis oil.

Another embodiment comprises cyclosporin A, Coladet BSB-P®, and Arachis oil.

Another embodiment comprises cyclosporin A, Customulse O-20®, and Arachis oil.

Another embodiment comprises cyclosporin A, Dehymuls E®, and Arachis oil.

Another embodiment comprises cyclosporin A, DeSotan SMO®, and Arachis oil.

Another embodiment comprises cyclosporin A, DeSotan SMO-20®, and Arachis oil.

Another embodiment comprises cyclosporin A, DeSotan SMT®, and Arachis oil.

Another embodiment comprises cyclosporin A, DeSotan SMT-20®, and Arachis oil.

Another embodiment comprises cyclosporin A, Durfax 60®, and Arachis oil.

Another embodiment comprises cyclosporin A, Durfax 65®, and Arachis oil.

Another embodiment comprises cyclosporin A, Durfax 80®, and Arachis oil.

Another embodiment comprises cyclosporin A, Durtan 60®, and Arachis oil.

Another embodiment comprises cyclosporin A, Durtan 65®, and Arachis oil.

Another embodiment comprises cyclosporin A, Liposorb L®, and Arachis oil.

Another embodiment comprises cyclosporin A, Liposorb L-10®, and Arachis oil.

Another embodiment comprises cyclosporin A, Liposorb L-20®, and Arachis oil.

Another embodiment comprises cyclosporin A, Liposorb O®, and Arachis oil.

Another embodiment comprises cyclosporin A, Liposorb O-20®, and Arachis oil.

Another embodiment comprises cyclosporin A, Liposorb P®, and Arachis oil.

Another embodiment comprises cyclosporin A, Liposorb P-20®, and Arachis oil.

Another embodiment comprises cyclosporin A, Liposorb S®, and Arachis oil.

Another embodiment comprises cyclosporin A, Liposorb S-20®, and Arachis oil.

Another embodiment comprises cyclosporin A, Liposorb SQO®, and Arachis oil.

Another embodiment comprises cyclosporin A, Liposorb TO®, and Arachis oil.

Another embodiment comprises cyclosporin A, Liposorb TS®, and Arachis oil.

Another embodiment comprises cyclosporin A, Liposorb TS-20®, and Arachis oil.

Another embodiment comprises cyclosporin A, Lumisorb PO®, and Arachis oil.

Another embodiment comprises cyclosporin A, Lumisorb SMO (T)®, and Arachis oil.

Another embodiment comprises cyclosporin A, Lumisorb SMS K®, and Arachis oil.

Another embodiment comprises cyclosporin A, Lumisorb SSO®, and Arachis oil.

Another embodiment comprises cyclosporin A, Lumisorb STS K®, and Arachis oil.

Another embodiment comprises cyclosporin A, Lumisorb STT®, and Arachis oil.

Another embodiment comprises cyclosporin A, Magrabar SMO®, and Arachis oil.

Another embodiment comprises cyclosporin A, Magrabar SMO-VEG®, and Arachis oil.

Another embodiment comprises cyclosporin A, Magrabar SMT®, and Arachis oil.

Another embodiment comprises cyclosporin A, Magrabar STO®, and Arachis oil.

Another embodiment comprises cyclosporin A, Miracare BC-27®, and Arachis oil.

Another embodiment comprises cyclosporin A, Ritabate 20®, and Arachis oil.

Another embodiment comprises cyclosporin A, Ritabate 40®, and Arachis oil.

Another embodiment comprises cyclosporin A, Ritabate 60®, and Arachis oil.

Another embodiment comprises cyclosporin A, Ritabate 80®, and Arachis oil.

Another embodiment comprises cyclosporin A, T-Maz®, and Arachis oil.

Another embodiment comprises cyclosporin A, Tego SML®, and Arachis oil.

Another embodiment comprises cyclosporin A, Tego SML 20®, and Arachis oil.

Another embodiment comprises cyclosporin A, Tego SMO 80 V®, and Arachis oil.

Another embodiment comprises cyclosporin A, Tego SMO V®, and Arachis oil.

Another embodiment comprises cyclosporin A, Tego SMS®, and Arachis oil.

Another embodiment comprises cyclosporin A, Tego STO V®, and Arachis oil.

Another embodiment comprises cyclosporin A, Tween 21®, and Arachis oil.

Another embodiment comprises cyclosporin A, Tween 40®, and Arachis oil.

Another embodiment comprises cyclosporin A, Tween 60®, and Arachis oil.

Another embodiment comprises cyclosporin A, Tween 60 K®, and Arachis oil.

Another embodiment comprises cyclosporin A, Tween 61®, and Arachis oil.

Another embodiment comprises cyclosporin A, Tween 65®, and Arachis oil.

Another embodiment comprises cyclosporin A, Tween 80®, and Arachis oil.

Another embodiment comprises cyclosporin A, Tween 80 K®, and Arachis oil.

Another embodiment comprises cyclosporin A, Tween 81®, and Arachis oil.

Another embodiment comprises cyclosporin A, Tween 85®, and Arachis oil.

Another embodiment comprises cyclosporin A, a Sucrose or Glucose Ester, or Derivative thereof, and Arachis oil.

Another embodiment comprises cyclosporin A, DeSulf GOS-P-60WCG®, and Arachis oil.

Another embodiment comprises cyclosporin A, Glucam E-20 Distearate®, and Arachis oil.

Another embodiment comprises cyclosporin A, Glucamate DOE-120®, and Arachis oil.

Another embodiment comprises cyclosporin A, Glucamate SSE-20®, and Arachis oil.

Another embodiment comprises cyclosporin A, Glucate DO®, and Arachis oil.

Another embodiment comprises cyclosporin A, Glucate SS®, and Arachis oil.

Another embodiment comprises cyclosporin A, Glucopon 425 UP®, and Arachis oil.

Another embodiment comprises cyclosporin A, Isolan IS®, and Arachis oil.

Another embodiment comprises cyclosporin A, Mazon 40®, and Arachis oil.

Another embodiment comprises cyclosporin A, Montanov 82®, and Arachis oil.

Another embodiment comprises cyclosporin A, Montanov 202®, and Arachis oil.

Another embodiment comprises cyclosporin A, Montanov S®, and Arachis oil.

Another embodiment comprises cyclosporin A, Rheozan®, and Arachis oil.

Another embodiment comprises cyclosporin A, Simulsol AS 48®, and Arachis oil.

Another embodiment comprises cyclosporin A, Simulsol SL 4®, and Arachis oil.

Another embodiment comprises cyclosporin A, Simulsol SL 10®, and Arachis oil.

Another embodiment comprises cyclosporin A, Simulsol SL 11W®, and Arachis oil.

Another embodiment comprises cyclosporin A, Simulsol SL 55®, and Arachis oil.

Another embodiment comprises cyclosporin A, Suga Nate 100 and 160®, and Arachis oil.

Another embodiment comprises cyclosporin A, Tego Care 450®, and Arachis oil.

Another embodiment comprises cyclosporin A, Tego Care CG 90®, and Arachis oil.

Another embodiment comprises cyclosporin A, Tego Care PS®, and Arachis oil.

Another embodiment comprises cyclosporin A, Tegosott PSE 141 G®, and Arachis oil.

Another embodiment comprises cyclosporin A, Tegotens G 826®, and Arachis oil.

Another embodiment comprises cyclosporin A, Triton BG-10 (70%)®, and Arachis oil.

Another embodiment comprises cyclosporin A, Triton CG-110 (60%)®, and Arachis oil.

Another embodiment comprises cyclosporin A, Wickenol 545®, and Arachis oil.

Another embodiment comprises cyclosporin A, an alcohol, and Cottonseed oil.

Another embodiment comprises cyclosporin A, Diglycerol®, and Cottonseed oil.

Another embodiment comprises cyclosporin A, Hetoxide GT-80®, and Cottonseed oil.

Another embodiment comprises cyclosporin A, Lexemul BEO®, and Cottonseed oil.

Another embodiment comprises cyclosporin A, Polyglycerol-3®, and Cottonseed oil.

Another embodiment comprises cyclosporin A, Redicote E Series®, and Cottonseed oil.

Another embodiment comprises cyclosporin A, Simulsol OX 1005L®, and Cottonseed oil.

Another embodiment comprises cyclosporin A, Stanfax 567®, and Cottonseed oil.

Another embodiment comprises cyclosporin A, TA-1618®, and Cottonseed oil.

Another embodiment comprises cyclosporin A, Witconol H-31A®, and Cottonseed oil.

Another embodiment comprises cyclosporin A, an amine oxide, and Cottonseed oil.

Another embodiment comprises cyclosporin A, AO-405®, and Cottonseed oil.

Another embodiment comprises cyclosporin A, AO-455®, and Cottonseed oil.

Another embodiment comprises cyclosporin A, AO 728 Special®, and Cottonseed oil.

Another embodiment comprises cyclosporin A, Barlox 12®, and Cottonseed oil.

Another embodiment comprises cyclosporin A, Barlox 14®, and Cottonseed oil.

Another embodiment comprises cyclosporin A, Burcoxide Lo®, and Cottonseed oil.

Another embodiment comprises cyclosporin A, Caloxamine LO®, and Cottonseed oil.

Another embodiment comprises cyclosporin A, Chemoxide CAW®, and Cottonseed oil.

Another embodiment comprises cyclosporin A, Chemoxide LM-30®, and Cottonseed oil.

Another embodiment comprises cyclosporin A, Chemoxide LO®, and Cottonseed oil.

Another embodiment comprises cyclosporin A, Chemoxide MO®, and Cottonseed oil.

Another embodiment comprises cyclosporin A, Colalux CAO-35®, and Cottonseed oil.

Another embodiment comprises cyclosporin A, Colalux LO®, and Cottonseed oil.

Another embodiment comprises cyclosporin A, DeMox CAPO®, and Cottonseed oil.

Another embodiment comprises cyclosporin A, DeMox CSG-30®, and Cottonseed oil.

Another embodiment comprises cyclosporin A, DeMox LAO®, and Cottonseed oil.

Another embodiment comprises cyclosporin A, Emcol L®, and Cottonseed oil.

Another embodiment comprises cyclosporin A, Empigen OB®, and Cottonseed oil.

Another embodiment comprises cyclosporin A, Empigen OS/A®, and Cottonseed oil.

Another embodiment comprises cyclosporin A, Foamox CDO®, and Cottonseed oil.

Another embodiment comprises cyclosporin A, Foamox DMM®, and Cottonseed oil.

Another embodiment comprises cyclosporin A, Foamox DMS®, and Cottonseed oil.

Another embodiment comprises cyclosporin A, Genaminox KC®, and Cottonseed oil.

Another embodiment comprises cyclosporin A, Genaminox LA®, and Cottonseed oil.

Another embodiment comprises cyclosporin A, Hartofoam SAO®, and Cottonseed oil.

Another embodiment comprises cyclosporin A, Hartox DMCD®, and Cottonseed oil.

Another embodiment comprises cyclosporin A, Lipowax DAT®, and Cottonseed oil.

Another embodiment comprises cyclosporin A, Lipowax PB Pastilles®, and Cottonseed oil.

Another embodiment comprises cyclosporin A, Mackamine C8®, and Cottonseed oil.

Another embodiment comprises cyclosporin A, Mackamine C10®, and Cottonseed oil.

Another embodiment comprises cyclosporin A, Mackamine C14®, and Cottonseed oil.

Another embodiment comprises cyclosporin A, Mackamine CAO®, and Cottonseed oil.

Another embodiment comprises cyclosporin A, Mackamine CO®, and Cottonseed oil.

Another embodiment comprises cyclosporin A, Mackamine LO®, and Cottonseed oil.

Another embodiment comprises cyclosporin A, Mackamine O2®, and Cottonseed oil.

Another embodiment comprises cyclosporin A, Mackamine SAO®, and Cottonseed oil.

Another embodiment comprises cyclosporin A, Mackamine SO®, and Cottonseed oil.

Another embodiment comprises cyclosporin A, Mazox KCAO®, and Cottonseed oil.

Another embodiment comprises cyclosporin A, Monalac MO®, and Cottonseed oil.

Another embodiment comprises cyclosporin A, Norfox LDA®, and Cottonseed oil.

Another embodiment comprises cyclosporin A, Rhodamox LO®, and Cottonseed oil.

Another embodiment comprises cyclosporin A, Schercamox C-AA®, and Cottonseed oil.

Another embodiment comprises cyclosporin A, Schercamox DMA®, and Cottonseed oil.

Another embodiment comprises cyclosporin A, Schercamox DML®, and Cottonseed oil.

Another embodiment comprises cyclosporin A, Schercamox DMM®, and Cottonseed oil.

Another embodiment comprises cyclosporin A, Schercamox DMS®, and Cottonseed oil.

Another embodiment comprises cyclosporin A, Tegotens DO®, and Cottonseed oil.

Another embodiment comprises cyclosporin A, Tomah AO-14-2®, and Cottonseed oil.

Another embodiment comprises cyclosporin A, Triaminox CDO®, and Cottonseed oil.

Another embodiment comprises cyclosporin A, a block polymer, and Cottonseed oil.

Another embodiment comprises cyclosporin A, AL 2070®, and Cottonseed oil.

Another embodiment comprises cyclosporin A, Antarox 17-R-2®, and Cottonseed oil.

Another embodiment comprises cyclosporin A, Antarox 25-R-2®, and Cottonseed oil.

Another embodiment comprises cyclosporin A, Antarox 31-R-1®, and Cottonseed oil.

Another embodiment comprises cyclosporin A, Antarox P-84®, and Cottonseed oil.

Another embodiment comprises cyclosporin A, Antarox P-104/H®, and Cottonseed oil.

Another embodiment comprises cyclosporin A, Arnox BP-Series®, and Cottonseed oil.

Another embodiment comprises cyclosporin A, Chemonic 435®, and Cottonseed oil.

Another embodiment comprises cyclosporin A, Chemonic D-25®, and Cottonseed oil.

Another embodiment comprises cyclosporin A, Chemonic PL Series®, and Cottonseed oil.

Another embodiment comprises cyclosporin A, Ethox L-121®, and Cottonseed oil.

Another embodiment comprises cyclosporin A, Ethox L-122®, and Cottonseed oil.

Another embodiment comprises cyclosporin A, Genapol PF-10®, and Cottonseed oil.

Another embodiment comprises cyclosporin A, Genapol PF-20®, and Cottonseed oil.

Another embodiment comprises cyclosporin A, Genapol PF-40A®, and Cottonseed oil.

Another embodiment comprises cyclosporin A, Nonox 2-LF®, and Cottonseed oil.

Another embodiment comprises cyclosporin A, Simulsol NW 342®, and Cottonseed oil.

Another embodiment comprises cyclosporin A, T-Det BP-1®, and Cottonseed oil.

Another embodiment comprises cyclosporin A, T-Det XD®, and Cottonseed oil.

Another embodiment comprises cyclosporin A, T-Det XH®, and Cottonseed oil.

Another embodiment comprises cyclosporin A, Triton CF-32®, and Cottonseed oil.

Another embodiment comprises cyclosporin A, Witconol 171®, and Cottonseed oil.

Another embodiment comprises cyclosporin A, Witconol 324®, and Cottonseed oil.

Another embodiment comprises cyclosporin A, Witconol 324D®, and Cottonseed oil.

Another embodiment comprises cyclosporin A, Witconol PD-2000®, and Cottonseed oil.

Another embodiment comprises cyclosporin A, a Carboxylated Alcohol or Alkylphenol Ethoxylate, and Cottonseed oil.

Another embodiment comprises cyclosporin A, Emcol CN-6®, and Cottonseed oil.

Another embodiment comprises cyclosporin A, Ethcarbs, and Cottonseed oil.

Another embodiment comprises cyclosporin A, Gemtex WNT-Conc®, and Cottonseed oil.

Another embodiment comprises cyclosporin A, Incrodet TD7-C®, and Cottonseed oil.

Another embodiment comprises cyclosporin A, Marlinat CM 105/80®, and Cottonseed oil.

Another embodiment comprises cyclosporin A, Marlowet 1072®, and Cottonseed oil.

Another embodiment comprises cyclosporin A, Marlowet 4530®, and Cottonseed oil.

Another embodiment comprises cyclosporin A, Marlowet 4530LF®, and Cottonseed oil.

Another embodiment comprises cyclosporin A, Marlowet 4534®, and Cottonseed oil.

Another embodiment comprises cyclosporin A, Marlowet 4538®, and Cottonseed oil.

Another embodiment comprises cyclosporin A, Marlowet 4539®, and Cottonseed oil.

Another embodiment comprises cyclosporin A, Marlowet 4539LF®, and Cottonseed oil.

Another embodiment comprises cyclosporin A, Marlowet 4541®, and Cottonseed oil.

Another embodiment comprises cyclosporin A, Miranate LEC-80®, and Cottonseed oil.

Another embodiment comprises cyclosporin A, Sandopan B®, and Cottonseed oil.

Another embodiment comprises cyclosporin A, Sandopan B Modified®, and Cottonseed oil.

Another embodiment comprises cyclosporin A, Sandopan LS-24 Gel®, and Cottonseed oil.

Another embodiment comprises cyclosporin A, Surfine T-A®, and Cottonseed oil.

Another embodiment comprises cyclosporin A, a Carboxylic Acid/Fatty Acid, and Cottonseed oil.

Another embodiment comprises cyclosporin A, Colaterge RAM®, and Cottonseed oil.

Another embodiment comprises cyclosporin A, Colatrope INC®, and Cottonseed oil.

Another embodiment comprises cyclosporin A, Crodacid B®, and Cottonseed oil.

Another embodiment comprises cyclosporin A, DeTrope CA-100®, and Cottonseed oil.

Another embodiment comprises cyclosporin A, Latol MTO®, and Cottonseed oil.

Another embodiment comprises cyclosporin A, Lumulse CC-33 K®, and Cottonseed oil.

Another embodiment comprises cyclosporin A, Mulls 2218®, and Cottonseed oil.

Another embodiment comprises cyclosporin A, OL-600®, and Cottonseed oil.

Another embodiment comprises cyclosporin A, OL-800®, and Cottonseed oil.

Another embodiment comprises cyclosporin A, R-910®, and Cottonseed oil.

Another embodiment comprises cyclosporin A, S-210®, and Cottonseed oil.

Another embodiment comprises cyclosporin A, Sandopan DTC Acid®, and Cottonseed oil.

Another embodiment comprises cyclosporin A, Sandopan LS 24 N®, and Cottonseed oil.

Another embodiment comprises cyclosporin A, Sandopan MA-18®, and Cottonseed oil.

Another embodiment comprises cyclosporin A, an Ethoxylated Alcohol, and Cottonseed oil.

Another embodiment comprises cyclosporin A, Adsee 799®, and Cottonseed oil.

Another embodiment comprises cyclosporin A, Adsee 799®, and Cottonseed oil.

Another embodiment comprises cyclosporin A, Alfonic 610-3.5®, and Cottonseed oil.

Another embodiment comprises cyclosporin A, Alfonic 810-2®, and Cottonseed oil.

Another embodiment comprises cyclosporin A, Alfonic 810-6®, and Cottonseed oil.

Another embodiment comprises cyclosporin A, Alfonic 1012-3®, and Cottonseed oil.

Another embodiment comprises cyclosporin A, Alfonic 1012-5®, and Cottonseed oil.

Another embodiment comprises cyclosporin A, Alfonic 1216CO-1.5®, and Cottonseed oil.

Another embodiment comprises cyclosporin A, Alfonic 1216CO-7®, and Cottonseed oil.

Another embodiment comprises cyclosporin A, Alfonic 1412-3®, and Cottonseed oil.

Another embodiment comprises cyclosporin A, Alfonic 1412-7®, and Cottonseed oil.

Another embodiment comprises cyclosporin A, Arlasolve 200®, and Cottonseed oil.

Another embodiment comprises cyclosporin A, Arlasolve 200 Liquid®, and Cottonseed oil.

Another embodiment comprises cyclosporin A, Armix 180-C®, and Cottonseed oil.

Another embodiment comprises cyclosporin A, Armix 183®, and Cottonseed oil.

Another embodiment comprises cyclosporin A, Armul 2404®, and Cottonseed oil.

Another embodiment comprises cyclosporin A, Armul 2404®, and Cottonseed oil.

Another embodiment comprises cyclosporin A, Atlas EMJ-C®, and Cottonseed oil.

Another embodiment comprises cyclosporin A, Atlas G-2109®, and Cottonseed oil.

Another embodiment comprises cyclosporin A, Atlas G-3886®, and Cottonseed oil.

Another embodiment comprises cyclosporin A, Atlas G-3890®, and Cottonseed oil.

Another embodiment comprises cyclosporin A, Bio Soft E-200®, and Cottonseed oil.

Another embodiment comprises cyclosporin A, Bio Soft E-300®, and Cottonseed oil.

Another embodiment comprises cyclosporin A, Bio Soft E-400®, and Cottonseed oil.

Another embodiment comprises cyclosporin A, Bio Soft EN 600®, and Cottonseed oil.

Another embodiment comprises cyclosporin A, Bio Soft TD-400®, and Cottonseed oil.

Another embodiment comprises cyclosporin A, Bio Soft TD-630®, and Cottonseed oil.

Another embodiment comprises cyclosporin A, Brij 30®, and Cottonseed oil.

Another embodiment comprises cyclosporin A, Brij 52®, and Cottonseed oil.

Another embodiment comprises cyclosporin A, Brij 56®, and Cottonseed oil.

Another embodiment comprises cyclosporin A, Brij 58®, and Cottonseed oil.

Another embodiment comprises cyclosporin A, Brij 72®, and Cottonseed oil.

Another embodiment comprises cyclosporin A, Brij 76®, and Cottonseed oil.

Another embodiment comprises cyclosporin A, Brij 78®, and Cottonseed oil.

Another embodiment comprises cyclosporin A, Brij 93®, and Cottonseed oil.

Another embodiment comprises cyclosporin A, Brij 97®, and Cottonseed oil.

Another embodiment comprises cyclosporin A, Brij 98®, and Cottonseed oil.

Another embodiment comprises cyclosporin A, Brij 700®, and Cottonseed oil.

Another embodiment comprises cyclosporin A, Brij 700 S®, and Cottonseed oil.

Another embodiment comprises cyclosporin A, Brij 721®, and Cottonseed oil.

Another embodiment comprises cyclosporin A, Brij 721 S®, and Cottonseed oil.

Another embodiment comprises cyclosporin A, Burcoterge CDG®, and Cottonseed oil.

Another embodiment comprises cyclosporin A, Canasol AT 600®, and Cottonseed oil.

Another embodiment comprises cyclosporin A, Canasol AT 800®, and Cottonseed oil.

Another embodiment comprises cyclosporin A, Canasol AT 1200®, and Cottonseed oil.

Another embodiment comprises cyclosporin A, Canasol BJ 35®, and Cottonseed oil.

Another embodiment comprises cyclosporin A, Canasol BJ 36®, and Cottonseed oil.

Another embodiment comprises cyclosporin A, Canasol BJ 52®, and Cottonseed oil.

Another embodiment comprises cyclosporin A, Canasol BJ 58®, and Cottonseed oil.

Another embodiment comprises cyclosporin A, Canasol BJ 72®, and Cottonseed oil.

Another embodiment comprises cyclosporin A, Canasol BJ 78®, and Cottonseed oil.

Another embodiment comprises cyclosporin A, Canasol BJ 98®, and Cottonseed oil.

Another embodiment comprises cyclosporin A, Canasol BJ 307®, and Cottonseed oil.

Another embodiment comprises cyclosporin A, Cenak 1400®, and Cottonseed oil.

Another embodiment comprises cyclosporin A, Cetomacrogol 1000 BP®, and Cottonseed oil.

Another embodiment comprises cyclosporin A, Chemonic C-2®, and Cottonseed oil.

Another embodiment comprises cyclosporin A, Chemonic C-10®, and Cottonseed oil.

Another embodiment comprises cyclosporin A, Chemonic C-20®, and Cottonseed oil.

Another embodiment comprises cyclosporin A, Chemonic CT-12®, and Cottonseed oil.

Another embodiment comprises cyclosporin A, Chemonic CT-20®, and Cottonseed oil.

Another embodiment comprises cyclosporin A, Chemonic CT-30®, and Cottonseed oil.

Another embodiment comprises cyclosporin A, Chemonic CT-55®, and Cottonseed oil.

Another embodiment comprises cyclosporin A, Chemonic G-7®, and Cottonseed oil.

Another embodiment comprises cyclosporin A, Chemonic G-26®, and Cottonseed oil.

Another embodiment comprises cyclosporin A, Chemonic L-4®, and Cottonseed oil.

Another embodiment comprises cyclosporin A, Chemonic L-7®, and Cottonseed oil.

Another embodiment comprises cyclosporin A, Chemonic L-12®, and Cottonseed oil.

Another embodiment comprises cyclosporin A, Chemonic L-23®, and Cottonseed oil.

Another embodiment comprises cyclosporin A, Chemonic O-2®, and Cottonseed oil.

Another embodiment comprises cyclosporin A, Chemonic O-5®, and Cottonseed oil.

Another embodiment comprises cyclosporin A, Chemonic O-10®, and Cottonseed oil.

Another embodiment comprises cyclosporin A, Chemonic O-20®, and Cottonseed oil.

Another embodiment comprises cyclosporin A, Chemonic S-2®, and Cottonseed oil.

Another embodiment comprises cyclosporin A, Chemonic S-10®, and Cottonseed oil.

Another embodiment comprises cyclosporin A, Chemonic S-20®, and Cottonseed oil.

Another embodiment comprises cyclosporin A, Colamulse FE®, and Cottonseed oil.

Another embodiment comprises cyclosporin A, Cremophor A 20®, and Cottonseed oil.

Another embodiment comprises cyclosporin A, Cremophor SA 2®, and Cottonseed oil.

Another embodiment comprises cyclosporin A, Dehydol 100®, and Cottonseed oil.

Another embodiment comprises cyclosporin A, Dehydol O-4®, and Cottonseed oil.

Another embodiment comprises cyclosporin A, Delonic C-18®, and Cottonseed oil.

Another embodiment comprises cyclosporin A, DeSonic 6T®, and Cottonseed oil.

Another embodiment comprises cyclosporin A, DeSonic 9D®, and Cottonseed oil.

Another embodiment comprises cyclosporin A, DeSonic 9T®, and Cottonseed oil.

Another embodiment comprises cyclosporin A, DeSonic 12D®, and Cottonseed oil.

Another embodiment comprises cyclosporin A, DeSonic 12T®, and Cottonseed oil.

Another embodiment comprises cyclosporin A, DeSonic 15T®, and Cottonseed oil.

Another embodiment comprises cyclosporin A, DeSonic TDA-9®, and Cottonseed oil.

Another embodiment comprises cyclosporin A, DeThox GLG-7®, and Cottonseed oil.

Another embodiment comprises cyclosporin A, DeThox GLG-26®, and Cottonseed oil.

Another embodiment comprises cyclosporin A, DeThox LA-4®, and Cottonseed oil.

Another embodiment comprises cyclosporin A, DeThox LA-23®, and Cottonseed oil.

Another embodiment comprises cyclosporin A, DeThox SA-80®, and Cottonseed oil.

Another embodiment comprises cyclosporin A, Disponil O05®, and Cottonseed oil.

Another embodiment comprises cyclosporin A, Eccoterge EO-41B®, and Cottonseed oil.

Another embodiment comprises cyclosporin A, Empilan KA2.5/90®, and Cottonseed oil.

Another embodiment comprises cyclosporin A, Empilan KA5/90®, and Cottonseed oil.

Another embodiment comprises cyclosporin A, Empilan KM-20®, and Cottonseed oil.

Another embodiment comprises cyclosporin A, Empilan KM-50®, and Cottonseed oil.

Another embodiment comprises cyclosporin A, Empilan L-23®, and Cottonseed oil.

Another embodiment comprises cyclosporin A, Ethylan 25-3®, and Cottonseed oil.

Another embodiment comprises cyclosporin A, Ethylan 1204®, and Cottonseed oil.

Another embodiment comprises cyclosporin A, Ethylan DA-4®, and Cottonseed oil.

Another embodiment comprises cyclosporin A, Ethylan LA-230®, and Cottonseed oil.

Another embodiment comprises cyclosporin A, Ethylan SN®, and Cottonseed oil.

Another embodiment comprises cyclosporin A, Ethylan TD-60®, and Cottonseed oil.

Another embodiment comprises cyclosporin A, Ethylan TD-100®, and Cottonseed oil.

Another embodiment comprises cyclosporin A, Ethylan TD-1407®, and Cottonseed oil.

Another embodiment comprises cyclosporin A, Eumulgin B1®, and Cottonseed oil.

Another embodiment comprises cyclosporin A, Eumulgin B2®, and Cottonseed oil.

Another embodiment comprises cyclosporin A, Eumulgin B3®, and Cottonseed oil.

Another embodiment comprises cyclosporin A, Eumulgin O-10®, and Cottonseed oil.

Another embodiment comprises cyclosporin A, Flo Mo 80/20®, and Cottonseed oil.

Another embodiment comprises cyclosporin A, Flo Mo Low Foam®, and Cottonseed oil.

Another embodiment comprises cyclosporin A, Forlan C-24®, and Cottonseed oil.

Another embodiment comprises cyclosporin A, Genapol 1454®, and Cottonseed oil.

Another embodiment comprises cyclosporin A, Genapol BA-020®, and Cottonseed oil.

Another embodiment comprises cyclosporin A, Genapol BA-040®, and Cottonseed oil.

Another embodiment comprises cyclosporin A, Genapol C-100®, and Cottonseed oil.

Another embodiment comprises cyclosporin A, Genapol DA 060®, and Cottonseed oil.

Another embodiment comprises cyclosporin A, Genapol HS 020®, and Cottonseed oil.

Another embodiment comprises cyclosporin A, Genapol HS 200®, and Cottonseed oil.

Another embodiment comprises cyclosporin A, Genapol ID-040®, and Cottonseed oil.

Another embodiment comprises cyclosporin A, Genapol ID-060®, and Cottonseed oil.

Another embodiment comprises cyclosporin A, Genapol ID-090®, and Cottonseed oil.

Another embodiment comprises cyclosporin A, Genapol LA 010®, and Cottonseed oil.

Another embodiment comprises cyclosporin A, Genapol LA 020®, and Cottonseed oil.

Another embodiment comprises cyclosporin A, Genapol LA 030®, and Cottonseed oil.

Another embodiment comprises cyclosporin A, Genapol LA 040®, and Cottonseed oil.

Another embodiment comprises cyclosporin A, Genapol LA 050®, and Cottonseed oil.

Another embodiment comprises cyclosporin A, Genapol LA 060®, and Cottonseed oil.

Another embodiment comprises cyclosporin A, Genapol LA 070®, and Cottonseed oil.

Another embodiment comprises cyclosporin A, Genapol LA 070S®, and Cottonseed oil.

Another embodiment comprises cyclosporin A, Genapol LA 230®, and Cottonseed oil.

Another embodiment comprises cyclosporin A, Genapol O020®, and Cottonseed oil.

Another embodiment comprises cyclosporin A, Genapol O050®, and Cottonseed oil.

Another embodiment comprises cyclosporin A, Genapol O100®, and Cottonseed oil.

Another embodiment comprises cyclosporin A, Genapol O200®, and Cottonseed oil.

Another embodiment comprises cyclosporin A, Genapol SA 030®, and Cottonseed oil.

Another embodiment comprises cyclosporin A, Genapol SA 120®, and Cottonseed oil.

Another embodiment comprises cyclosporin A, Genapol T-020®, and Cottonseed oil.

Another embodiment comprises cyclosporin A, Genapol UD-030®, and Cottonseed oil.

Another embodiment comprises cyclosporin A, Genapol UD-050®, and Cottonseed oil.

Another embodiment comprises cyclosporin A, Genapol UD-070®, and Cottonseed oil.

Another embodiment comprises cyclosporin A, Genapol UD-079®, and Cottonseed oil.

Another embodiment comprises cyclosporin A, Genapol UD-080®, and Cottonseed oil.

Another embodiment comprises cyclosporin A, Genapol UD-110®, and Cottonseed oil.

Another embodiment comprises cyclosporin A, Genapol X 030®, and Cottonseed oil.

Another embodiment comprises cyclosporin A, Genapol X 050®, and Cottonseed oil.

Another embodiment comprises cyclosporin A, Genapol X 060®, and Cottonseed oil.

Another embodiment comprises cyclosporin A, Genapol X 070®, and Cottonseed oil.

Another embodiment comprises cyclosporin A, Genapol X 080®, and Cottonseed oil.

Another embodiment comprises cyclosporin A, Genapol X 100®, and Cottonseed oil.

Another embodiment comprises cyclosporin A, Genapol X 159®, and Cottonseed oil.

Another embodiment comprises cyclosporin A, Generol 122 E5®, and Cottonseed oil.

Another embodiment comprises cyclosporin A, Generol 122 E25®, and Cottonseed oil.

Another embodiment comprises cyclosporin A, Hostacerin T-3®, and Cottonseed oil.

Another embodiment comprises cyclosporin A, Iconol LF 110®, and Cottonseed oil.

Another embodiment comprises cyclosporin A, Incropol CS-20®, and Cottonseed oil.

Another embodiment comprises cyclosporin A, Lexemul CS-20®, and Cottonseed oil.

Another embodiment comprises cyclosporin A, Liponic EG-1®, and Cottonseed oil.

Another embodiment comprises cyclosporin A, Lipowax D®, and Cottonseed oil.

Another embodiment comprises cyclosporin A, Lipowax G®, and Cottonseed oil.

Another embodiment comprises cyclosporin A, Lipowax NI®, and Cottonseed oil.

Another embodiment comprises cyclosporin A, Lipowax P®, and Cottonseed oil.

Another embodiment comprises cyclosporin A, Lipowax P-31®, and Cottonseed oil.

Another embodiment comprises cyclosporin A, Lipowax PR®, and Cottonseed oil.

Another embodiment comprises cyclosporin A, Lumulse CS-20®, and Cottonseed oil.

Another embodiment comprises cyclosporin A, Macol CSA-20®, and Cottonseed oil.

Another embodiment comprises cyclosporin A, Marlox B 24/50®, and Cottonseed oil.

Another embodiment comprises cyclosporin A, Mazawet 77®, and Cottonseed oil.

Another embodiment comprises cyclosporin A, Norlox 1713®, and Cottonseed oil.

Another embodiment comprises cyclosporin A, Norlox 2579®, and Cottonseed oil.

Another embodiment comprises cyclosporin A, Norfox Lo Foam®, and Cottonseed oil.

Another embodiment comprises cyclosporin A, Promulgen D®, and Cottonseed oil.

Another embodiment comprises cyclosporin A, Promulgen G®, and Cottonseed oil.

Another embodiment comprises cyclosporin A, Renex 30®, and Cottonseed oil.

Another embodiment comprises cyclosporin A, Renex 36®, and Cottonseed oil.

Another embodiment comprises cyclosporin A, Rhodasurf A 24®, and Cottonseed oil.

Another embodiment comprises cyclosporin A, Rhodasurf AAE-10®, and Cottonseed oil.

Another embodiment comprises cyclosporin A, Rhodasurf BEH-25®, and Cottonseed oil.

Another embodiment comprises cyclosporin A, Rhodasurf BEH-40®, and Cottonseed oil.

Another embodiment comprises cyclosporin A, Rhodasurf DA 530®, and Cottonseed oil.

Another embodiment comprises cyclosporin A, Rhodasurf DA 630®, and Cottonseed oil.

Another embodiment comprises cyclosporin A, Rhodasurf DA 639®, and Cottonseed oil.

Another embodiment comprises cyclosporin A, Rhodasurf LAN-23®, and Cottonseed oil.

Another embodiment comprises cyclosporin A, Rhodasurf ON-870®, and Cottonseed oil.

Another embodiment comprises cyclosporin A, Rhodasurf ON-877®, and Cottonseed oil.

Another embodiment comprises cyclosporin A, Rhodasurf TB-970 FLK®, and Cottonseed oil.

Another embodiment comprises cyclosporin A, Ritacet-20®, and Cottonseed oil.

Another embodiment comprises cyclosporin A, Ritachol 1000®, and Cottonseed oil.

Another embodiment comprises cyclosporin A, Ritachol 2000®, and Cottonseed oil.

Another embodiment comprises cyclosporin A, Ritachol 5000®, and Cottonseed oil.

Another embodiment comprises cyclosporin A, Ritox 35®, and Cottonseed oil.

Another embodiment comprises cyclosporin A, Surfonic DA-4®, and Cottonseed oil.

Another embodiment comprises cyclosporin A, Surfonic DA-6®, and Cottonseed oil.

Another embodiment comprises cyclosporin A, Surfonic L46-7®, and Cottonseed oil.

Another embodiment comprises cyclosporin A, Surfonic POA®, and Cottonseed oil.

Another embodiment comprises cyclosporin A, Synthrapol KB®, and Cottonseed oil.

Another embodiment comprises cyclosporin A, Teginacid®, and Cottonseed oil.

Another embodiment comprises cyclosporin A, Teginacid C®, and Cottonseed oil.

Another embodiment comprises cyclosporin A, Tegotens EC 11®, and Cottonseed oil.

Another embodiment comprises cyclosporin A, Tinegal NA®, and Cottonseed oil.

Another embodiment comprises cyclosporin A, Tomadol 400®, and Cottonseed oil.

Another embodiment comprises cyclosporin A, Tomadol 600®, and Cottonseed oil.

Another embodiment comprises cyclosporin A, Tomadol 900®, and Cottonseed oil.

Another embodiment comprises cyclosporin A, Uniperol O®, and Cottonseed oil.

Another embodiment comprises cyclosporin A, Witconol SN Series®, and Cottonseed oil.

Another embodiment comprises cyclosporin A, an Ethoxylated Alkylphenol, and Cottonseed oil.

Another embodiment comprises cyclosporin A, Antarox LF-222®, and Cottonseed oil.

Another embodiment comprises cyclosporin A, Atlox 775®, and Cottonseed oil.

Another embodiment comprises cyclosporin A, Caloxylate N-9®, and Cottonseed oil.

Another embodiment comprises cyclosporin A, Canasol NF-1000®, and Cottonseed oil.

Another embodiment comprises cyclosporin A, Canasol NF-3000®, and Cottonseed oil.

Another embodiment comprises cyclosporin A, Canasol NF-3070®, and Cottonseed oil.

Another embodiment comprises cyclosporin A, Canasol OF 1670®, and Cottonseed oil.

Another embodiment comprises cyclosporin A, Canasol OF 2570®, and Cottonseed oil.

Another embodiment comprises cyclosporin A, Canasol OF 4070®, and Cottonseed oil.

Another embodiment comprises cyclosporin A, Chemax DNP-8®, and Cottonseed oil.

Another embodiment comprises cyclosporin A, Chemax DNP-18®, and Cottonseed oil.

Another embodiment comprises cyclosporin A, Chemax DNP-150/50®, and Cottonseed oil.

Another embodiment comprises cyclosporin A, DeSonic 1.5N®, and Cottonseed oil.

Another embodiment comprises cyclosporin A, DeSonic 4N®, and Cottonseed oil.

Another embodiment comprises cyclosporin A, DeSonic 5N®, and Cottonseed oil.

Another embodiment comprises cyclosporin A, DeSonic 6D®, and Cottonseed oil.

Another embodiment comprises cyclosporin A, DeSonic 6N®, and Cottonseed oil.

Another embodiment comprises cyclosporin A, DeSonic 7N®, and Cottonseed oil.

Another embodiment comprises cyclosporin A, DeSonic 9N®, and Cottonseed oil.

Another embodiment comprises cyclosporin A, DeSonic 10D®, and Cottonseed oil.

Another embodiment comprises cyclosporin A, DeSonic 11N®, and Cottonseed oil.

Another embodiment comprises cyclosporin A, DeSonic 12N®, and Cottonseed oil.

Another embodiment comprises cyclosporin A, DeSonic 15N®, and Cottonseed oil.

Another embodiment comprises cyclosporin A, DeSonic 20N®, and Cottonseed oil.

Another embodiment comprises cyclosporin A, Eccoscour RC®, and Cottonseed oil.

Another embodiment comprises cyclosporin A, Eccoterge EO-100®, and Cottonseed oil.

Another embodiment comprises cyclosporin A, Emulsifier 632/90%®, and Cottonseed oil.

Another embodiment comprises cyclosporin A, Geronol AG-821®, and Cottonseed oil.

Another embodiment comprises cyclosporin A, Gradonic N-95®, and Cottonseed oil.

Another embodiment comprises cyclosporin A, Hetoxide NP-4®, and Cottonseed oil.

Another embodiment comprises cyclosporin A, Hetoxide NP-30®, and Cottonseed oil.

Another embodiment comprises cyclosporin A, Hostapal N-100®, and Cottonseed oil.

Another embodiment comprises cyclosporin A, Hostapal N-110®, and Cottonseed oil.

Another embodiment comprises cyclosporin A, Igepal CTA-639W®, and Cottonseed oil.

Another embodiment comprises cyclosporin A, Igepal DAP-9®, and Cottonseed oil.

Another embodiment comprises cyclosporin A, Igepal OD-410®, and Cottonseed oil.

Another embodiment comprises cyclosporin A, Igepal SS-837®, and Cottonseed oil.

Another embodiment comprises cyclosporin A, Lipocol NP-9 USP®, and Cottonseed oil.

Another embodiment comprises cyclosporin A, Macol DNP-10®, and Cottonseed oil.

Another embodiment comprises cyclosporin A, Marlophen NP 5®, and Cottonseed oil.

Another embodiment comprises cyclosporin A, Marlophen P 1®, and Cottonseed oil.

Another embodiment comprises cyclosporin A, Surfonic NB®, and Cottonseed oil.

Another embodiment comprises cyclosporin A, Surfonic OPB-307®, and Cottonseed oil.

Another embodiment comprises cyclosporin A, Surfonic OPB-407®, and Cottonseed oil.

Another embodiment comprises cyclosporin A, Syn Fac 334®, and Cottonseed oil.

Another embodiment comprises cyclosporin A, Syn Fac 8216®, and Cottonseed oil.

Another embodiment comprises cyclosporin A, Triton N-57®, and Cottonseed oil.

Another embodiment comprises cyclosporin A, Trycol 6956®, and Cottonseed oil.

Another embodiment comprises cyclosporin A, Trycol 6961®, and Cottonseed oil.

Another embodiment comprises cyclosporin A, Trycol 6964®, and Cottonseed oil.

Another embodiment comprises cyclosporin A, Trycol 6969®, and Cottonseed oil.

Another embodiment comprises cyclosporin A, Trycol 6974®, and Cottonseed oil.

Another embodiment comprises cyclosporin A, Witbreak DRB-127®, and Cottonseed oil.

Another embodiment comprises cyclosporin A, Witbreak DRB-127®, and Cottonseed oil.

Another embodiment comprises cyclosporin A, Witconol NP Series®, and Cottonseed oil.

Another embodiment comprises cyclosporin A, an Ethoxylated Arvl Phenol, and Cottonseed oil.

Another embodiment comprises cyclosporin A, Soprophor BSU®, and Cottonseed oil.

Another embodiment comprises cyclosporin A, Soprophor CY/8®, and Cottonseed oil.

Another embodiment comprises cyclosporin A, Soprophor S/25®, and Cottonseed oil.

Another embodiment comprises cyclosporin A, Witconol NIO®, and Cottonseed oil.

Another embodiment comprises cyclosporin A, Witconol NIW®, and Cottonseed oil.

Another embodiment comprises cyclosporin A, Witconol S-100®, and Cottonseed oil.

Another embodiment comprises cyclosporin A, an Ethoxvlated Fatty Acid, and Cottonseed oil.

Another embodiment comprises cyclosporin A, Aldo PGHMS®, and Cottonseed oil.

Another embodiment comprises cyclosporin A, Alkamuls TO-15/HR®, and Cottonseed oil.

Another embodiment comprises cyclosporin A, Armotan AL-69-66®, and Cottonseed oil.

Another embodiment comprises cyclosporin A, Cerasynt 840®, and Cottonseed oil.

Another embodiment comprises cyclosporin A, Cerasynt 945®, and Cottonseed oil.

Another embodiment comprises cyclosporin A, Crystal Inhibitor No. 5®, and Cottonseed oil.

Another embodiment comprises cyclosporin A, DeThox Acid L-9®, and Cottonseed oil.

Another embodiment comprises cyclosporin A, DeThox Acid S-8®, and Cottonseed oil.

Another embodiment comprises cyclosporin A, Ethofat 242/25®, and Cottonseed oil.

Another embodiment comprises cyclosporin A, Hydropalat 65®, and Cottonseed oil.

Another embodiment comprises cyclosporin A, Lipo EGMS®, and Cottonseed oil.

Another embodiment comprises cyclosporin A, Lipopeg 2 DL®, and Cottonseed oil.

Another embodiment comprises cyclosporin A, Lipopeg 4 DL®, and Cottonseed oil.

Another embodiment comprises cyclosporin A, Lipopeg 4-L®, and Cottonseed oil.

Another embodiment comprises cyclosporin A, Lipopeg 39-S®, and Cottonseed oil.

Another embodiment comprises cyclosporin A, Lipopeg 4-S®, and Cottonseed oil.

Another embodiment comprises cyclosporin A, Lipopeg 10-S®, and Cottonseed oil.

Another embodiment comprises cyclosporin A, Lipopeg 100-S®, and Cottonseed oil.

Another embodiment comprises cyclosporin A, Lipopeg 6000 DS®, and Cottonseed oil.

Another embodiment comprises cyclosporin A, Lumulse 40-L®, and Cottonseed oil.

Another embodiment comprises cyclosporin A, Lumulse 40-S®, and Cottonseed oil.

Another embodiment comprises cyclosporin A, Lumulse 42-L®, and Cottonseed oil.

Another embodiment comprises cyclosporin A, Lumulse 42-S®, and Cottonseed oil.

Another embodiment comprises cyclosporin A, Lumulse 100-S®, and Cottonseed oil.

Another embodiment comprises cyclosporin A, Lumulse 602-S®, and Cottonseed oil.

Another embodiment comprises cyclosporin A, Magrabar PGE-20-0®, and Cottonseed oil.

Another embodiment comprises cyclosporin A, Magrabar PGE-20L®, and Cottonseed oil.

Another embodiment comprises cyclosporin A, Magrabar PGE-20T®, and Cottonseed oil.

Another embodiment comprises cyclosporin A, Magrabar PGE-22-0®, and Cottonseed oil.

Another embodiment comprises cyclosporin A, Magrabar PGE-22L®, and Cottonseed oil.

Another embodiment comprises cyclosporin A, Magrabar PGE-22T®, and Cottonseed oil.

Another embodiment comprises cyclosporin A, Magrabar PGE-40-0®, and Cottonseed oil.

Another embodiment comprises cyclosporin A, Magrabar PGE-40L®, and Cottonseed oil.

Another embodiment comprises cyclosporin A, Magrabar PGE-40T®, and Cottonseed oil.

Another embodiment comprises cyclosporin A, Magrabar PGE-42-0®, and Cottonseed oil.

Another embodiment comprises cyclosporin A, Magrabar PGE-42L®, and Cottonseed oil.

Another embodiment comprises cyclosporin A, Magrabar PGE-42T®, and Cottonseed oil.

Another embodiment comprises cyclosporin A, Magrabar PGE-60-0®, and Cottonseed oil.

Another embodiment comprises cyclosporin A, Magrabar PGE-60L®, and Cottonseed oil.

Another embodiment comprises cyclosporin A, Magrabar PGE-60T®, and Cottonseed oil.

Another embodiment comprises cyclosporin A, Magrabar PGE-62-0®, and Cottonseed oil.

Another embodiment comprises cyclosporin A, Magrabar PGE-62L®, and Cottonseed oil.

Another embodiment comprises cyclosporin A, Magrabar PGE-62T®, and Cottonseed oil.

Another embodiment comprises cyclosporin A, Mapeg S-40K®, and Cottonseed oil.

Another embodiment comprises cyclosporin A, Marlowet OTS®, and Cottonseed oil.

Another embodiment comprises cyclosporin A, Naturechem PGR®, and Cottonseed oil.

Another embodiment comprises cyclosporin A, PG No. 4®, and Cottonseed oil.

Another embodiment comprises cyclosporin A, Renex 20®, and Cottonseed oil.

Another embodiment comprises cyclosporin A, Ritox 52®, and Cottonseed oil.

Another embodiment comprises cyclosporin A, Ritox 53®, and Cottonseed oil.

Another embodiment comprises cyclosporin A, Ritox 59®, and Cottonseed oil.

Another embodiment comprises cyclosporin A, Surfax 8916/A®, and Cottonseed oil.

Another embodiment comprises cyclosporin A, Tego Acid S 40 P®, and Cottonseed oil.

Another embodiment comprises cyclosporin A, Tego Acid S 100 P®, and Cottonseed oil.

Another embodiment comprises cyclosporin A, Tween 20®, and Cottonseed oil.

Another embodiment comprises cyclosporin A, Volpo 131®, and Cottonseed oil.

Another embodiment comprises cyclosporin A, an Ethoxylated Fatty Ester or Oil, and Cottonseed oil.

Another embodiment comprises cyclosporin A, Acconon 6-C10®, and Cottonseed oil.

Another embodiment comprises cyclosporin A, Acconon CC-6®, and Cottonseed oil.

Another embodiment comprises cyclosporin A, Acconon CO-7®, and Cottonseed oil.

Another embodiment comprises cyclosporin A, Aldosperse 40/60 FG®, and Cottonseed oil.

Another embodiment comprises cyclosporin A, Aldosperse ML-23®, and Cottonseed oil.

Another embodiment comprises cyclosporin A, Aldosperse MS-20 FG®, and Cottonseed oil.

Another embodiment comprises cyclosporin A, Alkamuls EL-620®, and Cottonseed oil.

Another embodiment comprises cyclosporin A, Alkamuls EL-719®, and Cottonseed oil.

Another embodiment comprises cyclosporin A, Alkamuls EL-985®, and Cottonseed oil.

Another embodiment comprises cyclosporin A, Arlatone G®, and Cottonseed oil.

Another embodiment comprises cyclosporin A, Arlatone T®, and Cottonseed oil.

Another embodiment comprises cyclosporin A, Atlas G-1045A®, and Cottonseed oil.

Another embodiment comprises cyclosporin A, Atlas G-1086®, and Cottonseed oil.

Another embodiment comprises cyclosporin A, Atlas G-1087®, and Cottonseed oil.

Another embodiment comprises cyclosporin A, Atlas G-1089®, and Cottonseed oil.

Another embodiment comprises cyclosporin A, Atlas G-1096®, and Cottonseed oil.

Another embodiment comprises cyclosporin A, Atlas G-1292®, and Cottonseed oil.

Another embodiment comprises cyclosporin A, Atlas G-1293®, and Cottonseed oil.

Another embodiment comprises cyclosporin A, Atlas G-1300®, and Cottonseed oil.

Another embodiment comprises cyclosporin A, Atlas G-7076®, and Cottonseed oil.

Another embodiment comprises cyclosporin A, Capmul EMG®, and Cottonseed oil.

Another embodiment comprises cyclosporin A, Chemonic CO-40®, and Cottonseed oil.

Another embodiment comprises cyclosporin A, Chemonic LI-3®, and Cottonseed oil.

Another embodiment comprises cyclosporin A, Chemonic LI-7®, and Cottonseed oil.

Another embodiment comprises cyclosporin A, Cirrasol GM®, and Cottonseed oil.

Another embodiment comprises cyclosporin A, Cremophor CO 40®, and Cottonseed oil.

Another embodiment comprises cyclosporin A, Cremophor CO 410®, and Cottonseed oil.

Another embodiment comprises cyclosporin A, Cremophor EL®, and Cottonseed oil.

Another embodiment comprises cyclosporin A, Cremophor GC7®, and Cottonseed oil.

Another embodiment comprises cyclosporin A, Cremophor RH-40®, and Cottonseed oil.

Another embodiment comprises cyclosporin A, Crovol A-40®, and Cottonseed oil.

Another embodiment comprises cyclosporin A, Crovol A-70®, and Cottonseed oil.

Another embodiment comprises cyclosporin A, Crovol M-70®, and Cottonseed oil.

Another embodiment comprises cyclosporin A, Crovol PK-70®, and Cottonseed oil.

Another embodiment comprises cyclosporin A, Cutina E-24®, and Cottonseed oil.

Another embodiment comprises cyclosporin A, Dacospin 12-R®, and Cottonseed oil.

Another embodiment comprises cyclosporin A, Dehymuls HRE-7®, and Cottonseed oil.

Another embodiment comprises cyclosporin A, DeSonic 30C®, and Cottonseed oil.

Another embodiment comprises cyclosporin A, DeSonic 36C®, and Cottonseed oil.

Another embodiment comprises cyclosporin A, DeSonic 40C®, and Cottonseed oil.

Another embodiment comprises cyclosporin A, Durfax 60®, and Cottonseed oil.

Another embodiment comprises cyclosporin A, Durfax 65®, and Cottonseed oil.

Another embodiment comprises cyclosporin A, Durfax 80®, and Cottonseed oil.

Another embodiment comprises cyclosporin A, Durfax EOM®, and Cottonseed oil.

Another embodiment comprises cyclosporin A, Eccoterge NF-2®, and Cottonseed oil.

Another embodiment comprises cyclosporin A, Emulpon CO-360®, and Cottonseed oil.

Another embodiment comprises cyclosporin A, Emulpon CO-550®, and Cottonseed oil.

Another embodiment comprises cyclosporin A, Emulsogen EL®, and Cottonseed oil.

Another embodiment comprises cyclosporin A, Emulsogen HCO 040®, and Cottonseed oil.

Another embodiment comprises cyclosporin A, Emulsogen HCO 060®, and Cottonseed oil.

Another embodiment comprises cyclosporin A, Emulsynt 1055®, and Cottonseed oil.

Another embodiment comprises cyclosporin A, Ethox 3095®, and Cottonseed oil.

Another embodiment comprises cyclosporin A, Eumulgin RO-40®, and Cottonseed oil.

Another embodiment comprises cyclosporin A, Genapol G-260®, and Cottonseed oil.

Another embodiment comprises cyclosporin A, Glycosperse L-20®, and Cottonseed oil.

Another embodiment comprises cyclosporin A, Glycosperse O-5®, and Cottonseed oil.

Another embodiment comprises cyclosporin A, Glycosperse O-20®, and Cottonseed oil.

Another embodiment comprises cyclosporin A, Glycosperse O-20 FG®, and Cottonseed oil.

Another embodiment comprises cyclosporin A, Glycosperse S-20®, and Cottonseed oil.

Another embodiment comprises cyclosporin A, Glycosperse S-20 FG®, and Cottonseed oil.

Another embodiment comprises cyclosporin A, Glycosperse TS-20®, and Cottonseed oil.

Another embodiment comprises cyclosporin A, Glycosperse TS-20 FG®, and Cottonseed oil.

Another embodiment comprises cyclosporin A, Hetan SL®, and Cottonseed oil.

Another embodiment comprises cyclosporin A, Hetan SO®, and Cottonseed oil.

Another embodiment comprises cyclosporin A, Hetan SS®, and Cottonseed oil.

Another embodiment comprises cyclosporin A, Hetoxide C-2®, and Cottonseed oil.

Another embodiment comprises cyclosporin A, Hetoxide C-9®, and Cottonseed oil.

Another embodiment comprises cyclosporin A, Hetoxide C-15®, and Cottonseed oil.

Another embodiment comprises cyclosporin A, Hetoxide C-25®, and Cottonseed oil.

Another embodiment comprises cyclosporin A, Hetoxide C-40®, and Cottonseed oil.

Another embodiment comprises cyclosporin A, Hetoxide C-200®, and Cottonseed oil.

Another embodiment comprises cyclosporin A, Hetoxide C-200-50%®, and Cottonseed oil.

Another embodiment comprises cyclosporin A, Hetoxide GC-30®, and Cottonseed oil.

Another embodiment comprises cyclosporin A, Hetoxide HC-60®, and Cottonseed oil.

Another embodiment comprises cyclosporin A, Ice No. 2®, and Cottonseed oil.

Another embodiment comprises cyclosporin A, Incrocas 30/40®, and Cottonseed oil.

Another embodiment comprises cyclosporin A, Lexol EC®, and Cottonseed oil.

Another embodiment comprises cyclosporin A, Lexol EO®, and Cottonseed oil.

Another embodiment comprises cyclosporin A, Lipocol HCO-40®, and Cottonseed oil.

Another embodiment comprises cyclosporin A, Lipocol HCO-60®, and Cottonseed oil.

Another embodiment comprises cyclosporin A, Lipocol O-3 Special®, and Cottonseed oil.

Another embodiment comprises cyclosporin A, Lipopeg 2-L®, and Cottonseed oil.

Another embodiment comprises cyclosporin A, Lipopeg 4-DO®, and Cottonseed oil.

Another embodiment comprises cyclosporin A, Lipopeg 4-DS®, and Cottonseed oil.

Another embodiment comprises cyclosporin A, Lipovol GTB®, and Cottonseed oil.

Another embodiment comprises cyclosporin A, Lonzest SML-20®, and Cottonseed oil.

Another embodiment comprises cyclosporin A, Lonzest SMO-20®, and Cottonseed oil.

Another embodiment comprises cyclosporin A, Lonzest SMS-20®, and Cottonseed oil.

Another embodiment comprises cyclosporin A, Lonzest STO-20®, and Cottonseed oil.

Another embodiment comprises cyclosporin A, Lonzest STS-20®, and Cottonseed oil.

Another embodiment comprises cyclosporin A, Lumulse GR-40®, and Cottonseed oil.

Another embodiment comprises cyclosporin A, Lumulse GRH-40®, and Cottonseed oil.

Another embodiment comprises cyclosporin A, Lumulse POE (7) GML®, and Cottonseed oil.

Another embodiment comprises cyclosporin A, Lumulse POE (12) Glyc®, and Cottonseed oil.

Another embodiment comprises cyclosporin A, Lumulse POE (40) MS KP®, and Cottonseed oil.

Another embodiment comprises cyclosporin A, Marlowet 4750®, and Cottonseed oil.

Another embodiment comprises cyclosporin A, Marlowet LVS®, and Cottonseed oil.

Another embodiment comprises cyclosporin A, Marlowet R 11®, and Cottonseed oil.

Another embodiment comprises cyclosporin A, Marlowet R 40®, and Cottonseed oil.

Another embodiment comprises cyclosporin A, Mazol 80 MGK®, and Cottonseed oil.

Another embodiment comprises cyclosporin A, Nonionic Emulsifier T-9®, and Cottonseed oil.

Another embodiment comprises cyclosporin A, Oronal LCG®, and Cottonseed oil.

Another embodiment comprises cyclosporin A, Polyderm PPI-CO-200®, and Cottonseed oil.

Another embodiment comprises cyclosporin A, Polyderm PPI-CO-40®, and Cottonseed oil.

Another embodiment comprises cyclosporin A, Rewoderm LI 520-70®, and Cottonseed oil.

Another embodiment comprises cyclosporin A, Ritapeg 150 DS®, and Cottonseed oil.

Another embodiment comprises cyclosporin A, Softigen 767®, and Cottonseed oil.

Another embodiment comprises cyclosporin A, Surfactol 318®, and Cottonseed oil.

Another embodiment comprises cyclosporin A, Surfactol 365®, and Cottonseed oil.

Another embodiment comprises cyclosporin A, Syn Lube 107®, and Cottonseed oil.

Another embodiment comprises cyclosporin A, Syn Lube 728®, and Cottonseed oil.

Another embodiment comprises cyclosporin A, Syn Lube 1632H®, and Cottonseed oil.

Another embodiment comprises cyclosporin A, Syn Lube 6277-A®, and Cottonseed oil.

Another embodiment comprises cyclosporin A, T-Det C-20®, and Cottonseed oil.

Another embodiment comprises cyclosporin A, T-Det C-40®, and Cottonseed oil.

Another embodiment comprises cyclosporin A, Tally 100 Plus®, and Cottonseed oil.

Another embodiment comprises cyclosporin A, Uniperol EL®, and Cottonseed oil.

Another embodiment comprises cyclosporin A, a Fatty Ester, and Cottonseed oil.

Another embodiment comprises cyclosporin A, Actralube-Syn 147®, and Cottonseed oil.

Another embodiment comprises cyclosporin A, Atlas G-1556®, and Cottonseed oil.

Another embodiment comprises cyclosporin A, Atlas G-1564®, and Cottonseed oil.

Another embodiment comprises cyclosporin A, Atlasol Base Oil S®, and Cottonseed oil.

Another embodiment comprises cyclosporin A, Base ML®, and Cottonseed oil.

Another embodiment comprises cyclosporin A, Base MT®, and Cottonseed oil.

Another embodiment comprises cyclosporin A, Cerasynt 303®, and Cottonseed oil.

Another embodiment comprises cyclosporin A, Dermol 1012®, and Cottonseed oil.

Another embodiment comprises cyclosporin A, Kemester 4000®, and Cottonseed oil.

Another embodiment comprises cyclosporin A, Lactipol S®, and Cottonseed oil.

Another embodiment comprises cyclosporin A, Magrabar PGO®, and Cottonseed oil.

Another embodiment comprises cyclosporin A, Mayco Base BFO®, and Cottonseed oil.

Another embodiment comprises cyclosporin A, Methyl Linoleate®, and Cottonseed oil.

Another embodiment comprises cyclosporin A, Pationic 122A®, and Cottonseed oil.

Another embodiment comprises cyclosporin A, Pationic 138C®, and Cottonseed oil.

Another embodiment comprises cyclosporin A, Pationic CSL®, and Cottonseed oil.

Another embodiment comprises cyclosporin A, Pationic ISL®, and Cottonseed oil.

Another embodiment comprises cyclosporin A, Pationic SBL®, and Cottonseed oil.

Another embodiment comprises cyclosporin A, Pationic SSL®, and Cottonseed oil.

Another embodiment comprises cyclosporin A, Ritasol®, and Cottonseed oil.

Another embodiment comprises cyclosporin A, Tego Alkanol CS 20®, and Cottonseed oil.

Another embodiment comprises cyclosporin A, Tego Alkanol L23 P®, and Cottonseed oil.

Another embodiment comprises cyclosporin A, Tego Alkanol S2®, and Cottonseed oil.

Another embodiment comprises cyclosporin A, Tego Alkanol S20 P®, and Cottonseed oil.

Another embodiment comprises cyclosporin A, Triemulsifier 600 MS®, and Cottonseed oil.

Another embodiment comprises cyclosporin A, a Glycerol Esters, and Cottonseed oil.

Another embodiment comprises cyclosporin A, Agro #9 Wint SBO®, and Cottonseed oil.

Another embodiment comprises cyclosporin A, Ahcovel Base 700®, and Cottonseed oil.

Another embodiment comprises cyclosporin A, Aldo HMS FG®, and Cottonseed oil.

Another embodiment comprises cyclosporin A, Aldo MLD®, and Cottonseed oil.

Another embodiment comprises cyclosporin A, Aldo MLD FG®, and Cottonseed oil.

Another embodiment comprises cyclosporin A, Aldo MO FG®, and Cottonseed oil.

Another embodiment comprises cyclosporin A, Aldo MS®, and Cottonseed oil.

Another embodiment comprises cyclosporin A, Aldo MS FG®, and Cottonseed oil.

Another embodiment comprises cyclosporin A, Aldo MS LG FG®, and Cottonseed oil.

Another embodiment comprises cyclosporin A, Aldo MSD®, and Cottonseed oil.

Another embodiment comprises cyclosporin A, Aldo MSD FG®, and Cottonseed oil.

Another embodiment comprises cyclosporin A, Aldosperse O-20 FG®, and Cottonseed oil.

Another embodiment comprises cyclosporin A, Aldosperse TS-20 FG®, and Cottonseed oil.

Another embodiment comprises cyclosporin A, Aldosperse TS-40 FG®, and Cottonseed oil.

Another embodiment comprises cyclosporin A, Arlacel 165®, and Cottonseed oil.

Another embodiment comprises cyclosporin A, Arlacel 186®, and Cottonseed oil.

Another embodiment comprises cyclosporin A, Capmul GMO®, and Cottonseed oil.

Another embodiment comprises cyclosporin A, Capmul GMS®, and Cottonseed oil.

Another embodiment comprises cyclosporin A, Caprol 3GO®, and Cottonseed oil.

Another embodiment comprises cyclosporin A, Caprol 3GVS®, and Cottonseed oil.

Another embodiment comprises cyclosporin A, Caprol 6G2S®, and Cottonseed oil.

Another embodiment comprises cyclosporin A, Caprol 10G40®, and Cottonseed oil.

Another embodiment comprises cyclosporin A, Caprol 10G100®, and Cottonseed oil.

Another embodiment comprises cyclosporin A, Caprol ET®, and Cottonseed oil.

Another embodiment comprises cyclosporin A, Caprol PGE860®, and Cottonseed oil.

Another embodiment comprises cyclosporin A, Cerasynt 945®, and Cottonseed oil.

Another embodiment comprises cyclosporin A, Cerasynt GMS®, and Cottonseed oil.

Another embodiment comprises cyclosporin A, Cerasynt Q®, and Cottonseed oil.

Another embodiment comprises cyclosporin A, Cerasynt SD®, and Cottonseed oil.

Another embodiment comprises cyclosporin A, Cerasynt WM®, and Cottonseed oil.

Another embodiment comprises cyclosporin A, Chemsperse 14®, and Cottonseed oil.

Another embodiment comprises cyclosporin A, Cremophor GO-32®, and Cottonseed oil.

Another embodiment comprises cyclosporin A, Cremophor GS11®, and Cottonseed oil.

Another embodiment comprises cyclosporin A, Cremophor GS-32®, and Cottonseed oil.

Another embodiment comprises cyclosporin A, Cutina KD-16®, and Cottonseed oil.

Another embodiment comprises cyclosporin A, Dehymuls PGPH®, and Cottonseed oil.

Another embodiment comprises cyclosporin A, Dermol DGDIS®, and Cottonseed oil.

Another embodiment comprises cyclosporin A, Dermol DGMIS®, and Cottonseed oil.

Another embodiment comprises cyclosporin A, Dermol G-76®, and Cottonseed oil.

Another embodiment comprises cyclosporin A, Dermol G-7DI®, and Cottonseed oil.

Another embodiment comprises cyclosporin A, Dermol NGDI®, and Cottonseed oil.

Another embodiment comprises cyclosporin A, Dermolan GLH®, and Cottonseed oil.

Another embodiment comprises cyclosporin A, Drewmulse GMO®, and Cottonseed oil.

Another embodiment comprises cyclosporin A, Drewpol 3-5-M®, and Cottonseed oil.

Another embodiment comprises cyclosporin A, Durlac 100 W®, and Cottonseed oil.

Another embodiment comprises cyclosporin A, Dur-Lo®, and Cottonseed oil.

Another embodiment comprises cyclosporin A, Dynasan 118®, and Cottonseed oil.

Another embodiment comprises cyclosporin A, EC-25®, and Cottonseed oil.

Another embodiment comprises cyclosporin A, EM 40®, and Cottonseed oil.

Another embodiment comprises cyclosporin A, Emerest 2400®, and Cottonseed oil.

Another embodiment comprises cyclosporin A, Emerest 2452®, and Cottonseed oil.

Another embodiment comprises cyclosporin A, Empilan G-26®, and Cottonseed oil.

Another embodiment comprises cyclosporin A, Genapol TSM®, and Cottonseed oil.

Another embodiment comprises cyclosporin A, Hostacerin DGI®, and Cottonseed oil.

Another embodiment comprises cyclosporin A, Hostacerin DGL®, and Cottonseed oil.

Another embodiment comprises cyclosporin A, Hostacerin DGMS®, and Cottonseed oil.

Another embodiment comprises cyclosporin A, Hostacerin DGSB®, and Cottonseed oil.

Another embodiment comprises cyclosporin A, Ice No. 2®, and Cottonseed oil.

Another embodiment comprises cyclosporin A, lmwitor 742®, and Cottonseed oil.

Another embodiment comprises cyclosporin A, lmwitor 780 K®, and Cottonseed oil.

Another embodiment comprises cyclosporin A, lmwitor 960 Flakes®, and Cottonseed oil.

Another embodiment comprises cyclosporin A, Isolan GI 34®, and Cottonseed oil.

Another embodiment comprises cyclosporin A, Isolan GO 33®, and Cottonseed oil.

Another embodiment comprises cyclosporin A, Kemester 1000®, and Cottonseed oil.

Another embodiment comprises cyclosporin A, Kemester 2000®, and Cottonseed oil.

Another embodiment comprises cyclosporin A, Kemester 2000®, and Cottonseed oil.

Another embodiment comprises cyclosporin A, Kemester 6000SE®, and Cottonseed oil.

Another embodiment comprises cyclosporin A, Lamecreme DGE 18®, and Cottonseed oil.

Another embodiment comprises cyclosporin A, Lexemul 515®, and Cottonseed oil.

Another embodiment comprises cyclosporin A, Lexemul 561®, and Cottonseed oil.

Another embodiment comprises cyclosporin A, Lexemul AR®, and Cottonseed oil.

Another embodiment comprises cyclosporin A, Lexemul AS®, and Cottonseed oil.

Another embodiment comprises cyclosporin A, Lexemul GDL®, and Cottonseed oil.

Another embodiment comprises cyclosporin A, Lexemul T®, and Cottonseed oil.

Another embodiment comprises cyclosporin A, Lipomulse 165®, and Cottonseed oil.

Another embodiment comprises cyclosporin A, Lumulse GML K®, and Cottonseed oil.

Another embodiment comprises cyclosporin A, Lumulse GMO K®, and Cottonseed oil.

Another embodiment comprises cyclosporin A, Lumulse GMR K®, and Cottonseed oil.

Another embodiment comprises cyclosporin A, Lumulse GMT K®, and Cottonseed oil.

Another embodiment comprises cyclosporin A, Magrabar GMC®, and Cottonseed oil.

Another embodiment comprises cyclosporin A, Magrabar GMO-CK®, and Cottonseed oil.

Another embodiment comprises cyclosporin A, Magrabar MDG-5050®, and Cottonseed oil.

Another embodiment comprises cyclosporin A, Magrabar PGO-315®, and Cottonseed oil.

Another embodiment comprises cyclosporin A, Magrabar PGO-1010®, and Cottonseed oil.

Another embodiment comprises cyclosporin A, Mazol 300K®, and Cottonseed oil.

Another embodiment comprises cyclosporin A, Mazol GMO-K®, and Cottonseed oil.

Another embodiment comprises cyclosporin A, Mazol GMS-K®, and Cottonseed oil.

Another embodiment comprises cyclosporin A, Mazol PG031 -K®, and Cottonseed oil.

Another embodiment comprises cyclosporin A, Miglyol 812®, and Cottonseed oil.

Another embodiment comprises cyclosporin A, Norfox 165C®, and Cottonseed oil.

Another embodiment comprises cyclosporin A, Schercemol GMIS®, and Cottonseed oil.

Another embodiment comprises cyclosporin A, Tegin®, and Cottonseed oil.

Another embodiment comprises cyclosporin A, Tegin 4100 Pellets®, and Cottonseed oil.

Another embodiment comprises cyclosporin A, Tegin M Pellets®, and Cottonseed oil.

Another embodiment comprises cyclosporin A, Tegin OV®, and Cottonseed oil.

Another embodiment comprises cyclosporin A, Teginacid H®, and Cottonseed oil.

Another embodiment comprises cyclosporin A, Tego Cosmo P813®, and Cottonseed oil.

Another embodiment comprises cyclosporin A, Wickenol 535®, and Cottonseed oil.

Another embodiment comprises cyclosporin A, Witconol 14®, and Cottonseed oil.

Another embodiment comprises cyclosporin A, Witconol 14®, and Cottonseed oil.

Another embodiment comprises cyclosporin A, Witconol 14F®, and Cottonseed oil.

Another embodiment comprises cyclosporin A, Witconol 18L®, and Cottonseed oil.

Another embodiment comprises cyclosporin A, Witconol GOT®, and Cottonseed oil.

Another embodiment comprises cyclosporin A, Witconol MST®, and Cottonseed oil.

Another embodiment comprises cyclosporin A, Witconol RHT®, and Cottonseed oil.

Another embodiment comprises cyclosporin A, a Glycol Ester, and Cottonseed oil.

Another embodiment comprises cyclosporin A, Alkamuls 600 DO®, and Cottonseed oil.

Another embodiment comprises cyclosporin A, Alkamuls SEG®, and Cottonseed oil.

Another embodiment comprises cyclosporin A, Atlas EM-2®, and Cottonseed oil.

Another embodiment comprises cyclosporin A, Cerasynt IP®, and Cottonseed oil.

Another embodiment comprises cyclosporin A, Cerasynt M®, and Cottonseed oil.

Another embodiment comprises cyclosporin A, Cerasynt MN®, and Cottonseed oil.

Another embodiment comprises cyclosporin A, Cerasynt PA®, and Cottonseed oil.

Another embodiment comprises cyclosporin A, Chemsperse EGDS®, and Cottonseed oil.

Another embodiment comprises cyclosporin A, Chemsperse EGMS®, and Cottonseed oil.

Another embodiment comprises cyclosporin A, Colonial Monolaurin®, and Cottonseed oil.

Another embodiment comprises cyclosporin A, DeMuls SGE-95®, and Cottonseed oil.

Another embodiment comprises cyclosporin A, Eccoterge 200®, and Cottonseed oil.

Another embodiment comprises cyclosporin A, Emerest 2380®, and Cottonseed oil.

Another embodiment comprises cyclosporin A, Ethox 2610®, and Cottonseed oil.

Another embodiment comprises cyclosporin A, Ethox DO-9®, and Cottonseed oil.

Another embodiment comprises cyclosporin A, Ethox DO-14®, and Cottonseed oil.

Another embodiment comprises cyclosporin A, Ethox SO-9®, and Cottonseed oil.

Another embodiment comprises cyclosporin A, Fizul MD-318®, and Cottonseed oil.

Another embodiment comprises cyclosporin A, Genapol EGDS-VHP®, and Cottonseed oil.

Another embodiment comprises cyclosporin A, Genapol TS Powder®, and Cottonseed oil.

Another embodiment comprises cyclosporin A, Hostacerin WO®, and Cottonseed oil.

Another embodiment comprises cyclosporin A, Inversol 140®, and Cottonseed oil.

Another embodiment comprises cyclosporin A, Kemester 104®, and Cottonseed oil.

Another embodiment comprises cyclosporin A, Kemester 205®, and Cottonseed oil.

Another embodiment comprises cyclosporin A, Kemester 226®, and Cottonseed oil.

Another embodiment comprises cyclosporin A, Kemester 5221SE®, and Cottonseed oil.

Another embodiment comprises cyclosporin A, Kemester EGDS®, and Cottonseed oil.

Another embodiment comprises cyclosporin A, Lexemul EGDS®, and Cottonseed oil.

Another embodiment comprises cyclosporin A, Lexemul EGMS®, and Cottonseed oil.

Another embodiment comprises cyclosporin A, Lexemul P®, and Cottonseed oil.

Another embodiment comprises cyclosporin A, Lipo DGLS®, and Cottonseed oil.

Another embodiment comprises cyclosporin A, Lipo EGDS®, and Cottonseed oil.

Another embodiment comprises cyclosporin A, Lipo PGMS®, and Cottonseed oil.

Another embodiment comprises cyclosporin A, Liposorb S-4®, and Cottonseed oil.

Another embodiment comprises cyclosporin A, Liposorb TO-20®, and Cottonseed oil.

Another embodiment comprises cyclosporin A, Lumulse PGO®, and Cottonseed oil.

Another embodiment comprises cyclosporin A, Mackester EGDS®, and Cottonseed oil.

Another embodiment comprises cyclosporin A, Mackester EGMS®, and Cottonseed oil.

Another embodiment comprises cyclosporin A, Mackester GSTP®, and Cottonseed oil.

Another embodiment comprises cyclosporin A, Mackester Series®, and Cottonseed oil.

Another embodiment comprises cyclosporin A, Magrabar PDG-50®, and Cottonseed oil.

Another embodiment comprises cyclosporin A, Mapeg 6000 DS®, and Cottonseed oil.

Another embodiment comprises cyclosporin A, Marlowet 4702®, and Cottonseed oil.

Another embodiment comprises cyclosporin A, Monalube 305®, and Cottonseed oil.

Another embodiment comprises cyclosporin A, Monalube 310®, and Cottonseed oil.

Another embodiment comprises cyclosporin A, Monalube 315®, and Cottonseed oil.

Another embodiment comprises cyclosporin A, Monalube 320®, and Cottonseed oil.

Another embodiment comprises cyclosporin A, Monalube 325®, and Cottonseed oil.

Another embodiment comprises cyclosporin A, Monalube 330®, and Cottonseed oil.

Another embodiment comprises cyclosporin A, Naturechem PGHS®, and Cottonseed oil.

Another embodiment comprises cyclosporin A, Polycastorol PLO-840®, and Cottonseed oil.

Another embodiment comprises cyclosporin A, Polytex 10OM®, and Cottonseed oil.

Another embodiment comprises cyclosporin A, Ritasynt IP®, and Cottonseed oil.

Another embodiment comprises cyclosporin A, Ross Chem PEG 600 DT®, and Cottonseed oil.

Another embodiment comprises cyclosporin A, Schercemol PGMS®, and Cottonseed oil.

Another embodiment comprises cyclosporin A, Sponto H-44C®, and Cottonseed oil.

Another embodiment comprises cyclosporin A, Tegin G®, and Cottonseed oil.

Another embodiment comprises cyclosporin A, Witbreak DGE-182®, and Cottonseed oil.

Another embodiment comprises cyclosporin A, Witbreak DGE-182®, and Cottonseed oil.

Another embodiment comprises cyclosporin A, Witbreak DRA-21®, and Cottonseed oil.

Another embodiment comprises cyclosporin A, Witbreak DRA-21®, and Cottonseed oil.

Another embodiment comprises cyclosporin A, Witbreak DRA-50®, and Cottonseed oil.

Another embodiment comprises cyclosporin A, Witbreak DRA-50®, and Cottonseed oil.

Another embodiment comprises cyclosporin A, Witconol F26-46®, and Cottonseed oil.

Another embodiment comprises cyclosporin A, Witconol H-32®, and Cottonseed oil.

Another embodiment comprises cyclosporin A, Witconol H-33®, and Cottonseed oil.

Another embodiment comprises cyclosporin A, Witconol H-35A®, and Cottonseed oil.

Another embodiment comprises cyclosporin A, Witconol RHP®, and Cottonseed oil.

Another embodiment comprises cyclosporin A, a Lanolin-based Derivative, and Cottonseed oil.

Another embodiment comprises cyclosporin A, Amerchol CAB®, and Cottonseed oil.

Another embodiment comprises cyclosporin A, Amerchol L-101®, and Cottonseed oil.

Another embodiment comprises cyclosporin A, Amerlate LFA-LO®, and Cottonseed oil.

Another embodiment comprises cyclosporin A, Amerlate P®, and Cottonseed oil.

Another embodiment comprises cyclosporin A, Barre Common Degras®, and Cottonseed oil.

Another embodiment comprises cyclosporin A, Cholesterol NF®, and Cottonseed oil.

Another embodiment comprises cyclosporin A, Crodalan AWS®, and Cottonseed oil.

Another embodiment comprises cyclosporin A, Crodalan LA®, and Cottonseed oil.

Another embodiment comprises cyclosporin A, Emery 1650®, and Cottonseed oil.

Another embodiment comprises cyclosporin A, Emery 1650®, and Cottonseed oil.

Another embodiment comprises cyclosporin A, Emery 1740®, and Cottonseed oil.

Another embodiment comprises cyclosporin A, Emery 1740®, and Cottonseed oil.

Another embodiment comprises cyclosporin A, Forlan 500®, and Cottonseed oil.

Another embodiment comprises cyclosporin A, Forlan L®, and Cottonseed oil.

Another embodiment comprises cyclosporin A, Laneto 50®, and Cottonseed oil.

Another embodiment comprises cyclosporin A, Laneto 100®, and Cottonseed oil.

Another embodiment comprises cyclosporin A, Laneto AWS®, and Cottonseed oil.

Another embodiment comprises cyclosporin A, Lanfrax 1776®, and Cottonseed oil.

Another embodiment comprises cyclosporin A, Lanfrax 1776®, and Cottonseed oil.

Another embodiment comprises cyclosporin A, Lanogel 21®, and Cottonseed oil.

Another embodiment comprises cyclosporin A, Lipolan®, and Cottonseed oil.

Another embodiment comprises cyclosporin A, Lipolan 31®, and Cottonseed oil.

Another embodiment comprises cyclosporin A, OHlan®, and Cottonseed oil.

Another embodiment comprises cyclosporin A, Polychol 5®, and Cottonseed oil.

Another embodiment comprises cyclosporin A, Polychol 15®, and Cottonseed oil.

Another embodiment comprises cyclosporin A, Ritacetyl®, and Cottonseed oil.

Another embodiment comprises cyclosporin A, Ritachol®, and Cottonseed oil.

Another embodiment comprises cyclosporin A, Ritahydrox®, and Cottonseed oil.

Another embodiment comprises cyclosporin A, Ritalafa®, and Cottonseed oil.

Another embodiment comprises cyclosporin A, Ritalan®, and Cottonseed oil.

Another embodiment comprises cyclosporin A, Ritalan AWS®, and Cottonseed oil.

Another embodiment comprises cyclosporin A, Ritalan C®, and Cottonseed oil.

Another embodiment comprises cyclosporin A, Ritawax®, and Cottonseed oil.

Another embodiment comprises cyclosporin A, Ritawax AEO®, and Cottonseed oil.

Another embodiment comprises cyclosporin A, Ritawax ALA®, and Cottonseed oil.

Another embodiment comprises cyclosporin A, Solan/Solan 50/Super Solan®, and Cottonseed oil.

Another embodiment comprises cyclosporin A, Super Hartolan/Hartolan®, and Cottonseed oil.

Another embodiment comprises cyclosporin A, Supersat AWS-4®, and Cottonseed oil.

Another embodiment comprises cyclosporin A, Supersat AWS-24®, and Cottonseed oil.

Another embodiment comprises cyclosporin A, a Lecithin or Lecithin Derivative, and Cottonseed oil.

Another embodiment comprises cyclosporin A, Alcolec®, and Cottonseed oil.

Another embodiment comprises cyclosporin A, Lecithin®, and Cottonseed oil.

Another embodiment comprises cyclosporin A, Lexin K®, and Cottonseed oil.

Another embodiment comprises cyclosporin A, Natipide®, and Cottonseed oil.

Another embodiment comprises cyclosporin A, a Lignin or Lignin Derivative, and Cottonseed oil.

Another embodiment comprises cyclosporin A, Diwatex XP 9®, and Cottonseed oil.

Another embodiment comprises cyclosporin A, Dynasperse LCD®, and Cottonseed oil.

Another embodiment comprises cyclosporin A, Indulin SAL®, and Cottonseed oil.

Another embodiment comprises cyclosporin A, Indulin W-1®, and Cottonseed oil.

Another embodiment comprises cyclosporin A, Indulin W-5®, and Cottonseed oil.

Another embodiment comprises cyclosporin A, Lignosol FTA®, and Cottonseed oil.

Another embodiment comprises cyclosporin A, Lignosol SFX-65®, and Cottonseed oil.

Another embodiment comprises cyclosporin A, Marasperse 52 CP®, and Cottonseed oil.

Another embodiment comprises cyclosporin A, Marasperse AG®, and Cottonseed oil.

Another embodiment comprises cyclosporin A, Marasperse CBOS-4®, and Cottonseed oil.

Another embodiment comprises cyclosporin A, Ufoxane 2®, and Cottonseed oil.

Another embodiment comprises cyclosporin A, a Methyl Ester, and Cottonseed oil.

Another embodiment comprises cyclosporin A, E.B. Cleaner AK®, and Cottonseed oil.

Another embodiment comprises cyclosporin A, Oleocal ME-70®, and Cottonseed oil.

Another embodiment comprises cyclosporin A, Trade Name Company®, and Cottonseed oil.

Another embodiment comprises cyclosporin A, Methyl Esters (cont'd)®, and Cottonseed oil.

Another embodiment comprises cyclosporin A, Oleocal ME-92®, and Cottonseed oil.

Another embodiment comprises cyclosporin A, Oleocal ME-112®, and Cottonseed oil.

Another embodiment comprises cyclosporin A, Oleocal ME-130®, and Cottonseed oil.

Another embodiment comprises cyclosporin A, a Monocilyceride or a Derivative thereof, and Cottonseed oil.

Another embodiment comprises cyclosporin A, Dynacet 211®, and Cottonseed oil.

Another embodiment comprises cyclosporin A, Hetsorb S-20®, and Cottonseed oil.

Another embodiment comprises cyclosporin A, lmwitor 191®, and Cottonseed oil.

Another embodiment comprises cyclosporin A, lmwitor 370®, and Cottonseed oil.

Another embodiment comprises cyclosporin A, lmwitor 375®, and Cottonseed oil.

Another embodiment comprises cyclosporin A, lmwitor 900®, and Cottonseed oil.

Another embodiment comprises cyclosporin A, lmwitor 945®, and Cottonseed oil.

Another embodiment comprises cyclosporin A, lmwitor 2020®, and Cottonseed oil.

Another embodiment comprises cyclosporin A, Kemester 5500®, and Cottonseed oil.

Another embodiment comprises cyclosporin A, Kemester 6000®, and Cottonseed oil.

Another embodiment comprises cyclosporin A, Magrabar GMC®, and Cottonseed oil.

Another embodiment comprises cyclosporin A, Magrabar GMO-CK®, and Cottonseed oil.

Another embodiment comprises cyclosporin A, Magrabar GPC-10®, and Cottonseed oil.

Another embodiment comprises cyclosporin A, Magrabar MDG-5050®, and Cottonseed oil.

Another embodiment comprises cyclosporin A, Monalube 335®, and Cottonseed oil.

Another embodiment comprises cyclosporin A, Monoglycerides Glyceryl Monestearate Archer Daniels Midland Company®, and Cottonseed oil.

Another embodiment comprises cyclosporin A, Rita GMS®, and Cottonseed oil.

Another embodiment comprises cyclosporin A, Ritamulse SCG®, and Cottonseed oil.

Another embodiment comprises cyclosporin A, Softigen 701®, and Cottonseed oil.

Another embodiment comprises cyclosporin A, Tally 100 Plus®, and Cottonseed oil.

Another embodiment comprises cyclosporin A, a Polyethylene Glycol. and Cottonseed oil.

Another embodiment comprises cyclosporin A, Emulgade PL 68/50®, and Cottonseed oil.

Another embodiment comprises cyclosporin A, Lumulse PEG®, and Cottonseed oil.

Another embodiment comprises cyclosporin A, Rhodasurf PEG-400®, and Cottonseed oil.

Another embodiment comprises cyclosporin A, Rhodasurf PEG-600®, and Cottonseed oil.

Another embodiment comprises cyclosporin A, Witconol PEG-400®, and Cottonseed oil.

Another embodiment comprises cyclosporin A, a Polymeric Surfactant. and Cottonseed oil.

Another embodiment comprises cyclosporin A, Acritamer PNC-EG®, and Cottonseed oil.

Another embodiment comprises cyclosporin A, Ag-Rho DEP-775®, and Cottonseed oil.

Another embodiment comprises cyclosporin A, APG 325N Glycoside®, and Cottonseed oil.

Another embodiment comprises cyclosporin A, Aristoflex AVC®, and Cottonseed oil.

Another embodiment comprises cyclosporin A, Aristoflex HMB®, and Cottonseed oil.

Another embodiment comprises cyclosporin A, Burco NPS-225®, and Cottonseed oil.

Another embodiment comprises cyclosporin A, Burco NPS-816®, and Cottonseed oil.

Another embodiment comprises cyclosporin A, Chemccinate 5603®, and Cottonseed oil.

Another embodiment comprises cyclosporin A, Cosmedia Guar C-261N®, and Cottonseed oil.

Another embodiment comprises cyclosporin A, Gantrez S-95®, and Cottonseed oil.

Another embodiment comprises cyclosporin A, Glucopon 220 UP®, and Cottonseed oil.

Another embodiment comprises cyclosporin A, Glucopon 225 DK®, and Cottonseed oil.

Another embodiment comprises cyclosporin A, Glucopon 425 N®, and Cottonseed oil.

Another embodiment comprises cyclosporin A, Glucopon 600 UP®, and Cottonseed oil.

Another embodiment comprises cyclosporin A, Glucopon 625 UP®, and Cottonseed oil.

Another embodiment comprises cyclosporin A, Pemulen 1621®, and Cottonseed oil.

Another embodiment comprises cyclosporin A, Pemulen 1622®, and Cottonseed oil.

Another embodiment comprises cyclosporin A, Pemulen TR-1®, and Cottonseed oil.

Another embodiment comprises cyclosporin A, Pemulen TR-2®, and Cottonseed oil.

Another embodiment comprises cyclosporin A, Plantacare 818®, and Cottonseed oil.

Another embodiment comprises cyclosporin A, Plantapon LGC Sorb®, and Cottonseed oil.

Another embodiment comprises cyclosporin A, Plantaren 1200N®, and Cottonseed oil.

Another embodiment comprises cyclosporin A, Plantaren 2000N®, and Cottonseed oil.

Another embodiment comprises cyclosporin A, Viscolam AT 64®, and Cottonseed oil.

Another embodiment comprises cyclosporin A, Viscolam AT 64P®, and Cottonseed oil.

Another embodiment comprises cyclosporin A, Viscolam AT 100®, and Cottonseed oil.

Another embodiment comprises cyclosporin A, Viscolam MAC 7®, and Cottonseed oil.

Another embodiment comprises cyclosporin A, Viscolam SMC 20®, and Cottonseed oil.

Another embodiment comprises cyclosporin A, Witbreak RTC-323®, and Cottonseed oil.

Another embodiment comprises cyclosporin A, WSI 3700®, and Cottonseed oil.

Another embodiment comprises cyclosporin A, a Propoxylated or Ethoxylated Fatty Acid, Alcohol, or Alkyl Phenol, and Cottonseed oil.

Another embodiment comprises cyclosporin A, Antarox AA-60®, and Cottonseed oil.

Another embodiment comprises cyclosporin A, Antarox LF-224®, and Cottonseed oil.

Another embodiment comprises cyclosporin A, Burcomul DFE-45®, and Cottonseed oil.

Another embodiment comprises cyclosporin A, Burcoterge LFE-1000®, and Cottonseed oil.

Another embodiment comprises cyclosporin A, Chemal LF-25B®, and Cottonseed oil.

Another embodiment comprises cyclosporin A, Chemal LF-40B®, and Cottonseed oil.

Another embodiment comprises cyclosporin A, Dehypon LS-36®, and Cottonseed oil.

Another embodiment comprises cyclosporin A, Dehypon LS-36®, and Cottonseed oil.

Another embodiment comprises cyclosporin A, Dehypon LS-54®, and Cottonseed oil.

Another embodiment comprises cyclosporin A, Dehypon LS-54®, and Cottonseed oil.

Another embodiment comprises cyclosporin A, Delonic 100 VLF®, and Cottonseed oil.

Another embodiment comprises cyclosporin A, Delonic LF-60 MOD®, and Cottonseed oil.

Another embodiment comprises cyclosporin A, Empiderm B®, and Cottonseed oil.

Another embodiment comprises cyclosporin A, Ethylan 1206®, and Cottonseed oil.

Another embodiment comprises cyclosporin A, Ethylan NS-500K®, and Cottonseed oil.

Another embodiment comprises cyclosporin A, Ethylan NS-500LQ®, and Cottonseed oil.

Another embodiment comprises cyclosporin A, Genapol 1392®, and Cottonseed oil.

Another embodiment comprises cyclosporin A, Genapol 2317®, and Cottonseed oil.

Another embodiment comprises cyclosporin A, Genapol 26EP710®, and Cottonseed oil.

Another embodiment comprises cyclosporin A, Genapol EP 1022®, and Cottonseed oil.

Another embodiment comprises cyclosporin A, Genapol EP 1024®, and Cottonseed oil.

Another embodiment comprises cyclosporin A, Genapol EP 6068®, and Cottonseed oil.

Another embodiment comprises cyclosporin A, Genapol NP915®, and Cottonseed oil.

Another embodiment comprises cyclosporin A, Kieralon MFB®, and Cottonseed oil.

Another embodiment comprises cyclosporin A, Lumisolve CSA-80 V®, and Cottonseed oil.

Another embodiment comprises cyclosporin A, Marlowet 5001®, and Cottonseed oil.

Another embodiment comprises cyclosporin A, Marlox FK 64®, and Cottonseed oil.

Another embodiment comprises cyclosporin A, Marlox MO 124®, and Cottonseed oil.

Another embodiment comprises cyclosporin A, Marlox S 58®, and Cottonseed oil.

Another embodiment comprises cyclosporin A, Nonatell 1003®, and Cottonseed oil.

Another embodiment comprises cyclosporin A, Nonatell 1038®, and Cottonseed oil.

Another embodiment comprises cyclosporin A, Nonatell 1052®, and Cottonseed oil.

Another embodiment comprises cyclosporin A, Nonatell 1061®, and Cottonseed oil.

Another embodiment comprises cyclosporin A, Nonatell 1075®, and Cottonseed oil.

Another embodiment comprises cyclosporin A, Nonatell 1088®, and Cottonseed oil.

Another embodiment comprises cyclosporin A, Nonatell 1123®, and Cottonseed oil.

Another embodiment comprises cyclosporin A, Nonatell 1153®, and Cottonseed oil.

Another embodiment comprises cyclosporin A, Nonatell 1161®, and Cottonseed oil.

Another embodiment comprises cyclosporin A, Nonatell 1172®, and Cottonseed oil.

Another embodiment comprises cyclosporin A, Nonatell 1181®, and Cottonseed oil.

Another embodiment comprises cyclosporin A, Norfox 36®, and Cottonseed oil.

Another embodiment comprises cyclosporin A, Procetyl AWS®, and Cottonseed oil.

Another embodiment comprises cyclosporin A, Sandoxylate SX 412®, and Cottonseed oil.

Another embodiment comprises cyclosporin A, Sandoxylate SX 418®, and Cottonseed oil.

Another embodiment comprises cyclosporin A, Sudonic JL-80X®, and Cottonseed oil.

Another embodiment comprises cyclosporin A, Surfonic JL-80X-B1®, and Cottonseed oil.

Another embodiment comprises cyclosporin A, Surfonic L4-29X®, and Cottonseed oil.

Another embodiment comprises cyclosporin A, Surfonic LF®, and Cottonseed oil.

Another embodiment comprises cyclosporin A, T-Det A826®, and Cottonseed oil.

Another embodiment comprises cyclosporin A, T-Det LF-416®, and Cottonseed oil.

Another embodiment comprises cyclosporin A, Tergitol Min-Foam 1X®, and Cottonseed oil.

Another embodiment comprises cyclosporin A, Tergitol Min-Foam 2X®, and Cottonseed oil.

Another embodiment comprises cyclosporin A, Triton CF-21®, and Cottonseed oil.

Another embodiment comprises cyclosporin A, Triton CF-76®, and Cottonseed oil.

Another embodiment comprises cyclosporin A, Triton XL-80N®, and Cottonseed oil.

Another embodiment comprises cyclosporin A, Witconol NS-98®, and Cottonseed oil.

Another embodiment comprises cyclosporin A, Witconol NS-108LQ®, and Cottonseed oil.

Another embodiment comprises cyclosporin A, Witconol NS-145®, and Cottonseed oil.

Another embodiment comprises cyclosporin A, Witconol NS-179®, and Cottonseed oil.

Another embodiment comprises cyclosporin A, a Protein-based Surfactant, and Cottonseed oil.

Another embodiment comprises cyclosporin A, AminoFoam W®, and.Cottonseed oil.

Another embodiment comprises cyclosporin A, Amiter LGOD-2®, and Cottonseed oil.

Another embodiment comprises cyclosporin A, Amiter LGS-2®, and Cottonseed oil.

Another embodiment comprises cyclosporin A, Amiter LGS-5®, and Cottonseed oil.

Another embodiment comprises cyclosporin A, Lamepon S®, and Cottonseed oil.

Another embodiment comprises cyclosporin A, Lamepon S®, and Cottonseed oil.

Another embodiment comprises cyclosporin A, Maypon 4C®, and Cottonseed oil.

Another embodiment comprises cyclosporin A, May-Tein C®, and Cottonseed oil.

Another embodiment comprises cyclosporin A, May-Tein CT®, and Cottonseed oil.

Another embodiment comprises cyclosporin A, May-Tein KTS®, and Cottonseed oil.

Another embodiment comprises cyclosporin A, May-Tein SY®, and Cottonseed oil.

Another embodiment comprises cyclosporin A, Plantapon S®, and Cottonseed oil.

Another embodiment comprises cyclosporin A, Proteol APL®, and Cottonseed oil.

Another embodiment comprises cyclosporin A, Proteol OAT®, and Cottonseed oil.

Another embodiment comprises cyclosporin A, Pyroter CPI-40®, and Cottonseed oil.

Another embodiment comprises cyclosporin A, Pyroter GPI-25®, and Cottonseed oil.

Another embodiment comprises cyclosporin A, Supro-Tein S®, and Cottonseed oil.

Another embodiment comprises cyclosporin A, Supro-Tein V®, and Cottonseed oil.

Another embodiment comprises cyclosporin A,® and Cottonseed oil.

Another embodiment comprises cyclosporin A, a Sarcosine Derivatives, and Cottonseed oil.

Another embodiment comprises cyclosporin A, Crodasinic LS-30®, and Cottonseed oil.

Another embodiment comprises cyclosporin A, Vanseal CS®, and Cottonseed oil.

Another embodiment comprises cyclosporin A, Vanseal LS®, and Cottonseed oil.

Another embodiment comprises cyclosporin A, Vanseal MS®, and Cottonseed oil.

Another embodiment comprises cyclosporin A, Vanseal NACS-30®, and Cottonseed oil.

Another embodiment comprises cyclosporin A, Vanseal NALS-95®, and Cottonseed oil.

Another embodiment comprises cyclosporin A, Vanseal OS®, and Cottonseed oil.

Another embodiment comprises cyclosporin A,® and Cottonseed oil.

Another embodiment comprises cyclosporin A, a Silicone-based Surfactant®, and Cottonseed oil.

Another embodiment comprises cyclosporin A, Abil-B-9950®, and Cottonseed oil.

Another embodiment comprises cyclosporin A, Abil Care 85®, and Cottonseed oil.

Another embodiment comprises cyclosporin A, Abil EM 90®, and Cottonseed oil.

Another embodiment comprises cyclosporin A, Abil EM 97®, and Cottonseed oil.

Another embodiment comprises cyclosporin A, Abil WE-09®, and Cottonseed oil.

Another embodiment comprises cyclosporin A, Dow Corning 1248 Fluid®, and Cottonseed oil.

Another embodiment comprises cyclosporin A, Dow Corning 3225C®, and Cottonseed oil.

Another embodiment comprises cyclosporin A, Dow Corning 5200®, and Cottonseed oil.

Another embodiment comprises cyclosporin A, Dow Corning Q4-3667®, and Cottonseed oil.

Another embodiment comprises cyclosporin A, Monasil PCA®, and Cottonseed oil.

Another embodiment comprises cyclosporin A, Monasil PDM®, and Cottonseed oil.

Another embodiment comprises cyclosporin A, Monasil PLN®, and Cottonseed oil.

Another embodiment comprises cyclosporin A, Polyderm PPI-SI-WS®, and Cottonseed oil.

Another embodiment comprises cyclosporin A, Troysol 380W®, and Cottonseed oil.

Another embodiment comprises cyclosporin A, Troysol S366®, and Cottonseed oil.

Another embodiment comprises cyclosporin A, a Sorbitan Derivative, and Cottonseed oil.

Another embodiment comprises cyclosporin A, Alkamuls SML®, and Cottonseed oil.

Another embodiment comprises cyclosporin A, Alkamuls SMO®, and Cottonseed oil.

Another embodiment comprises cyclosporin A, Alkamuls STO®, and Cottonseed oil.

Another embodiment comprises cyclosporin A, Arlacel 20®, and Cottonseed oil.

Another embodiment comprises cyclosporin A, Arlacel 40®, and Cottonseed oil.

Another embodiment comprises cyclosporin A, Arlacel 60®, and Cottonseed oil.

Another embodiment comprises cyclosporin A, Arlacel 80®, and Cottonseed oil.

Another embodiment comprises cyclosporin A, Arlacel C®, and Cottonseed oil.

Another embodiment comprises cyclosporin A, Armul 21®, and Cottonseed oil.

Another embodiment comprises cyclosporin A, Atlox 80®, and Cottonseed oil.

Another embodiment comprises cyclosporin A, Atlox 847®, and Cottonseed oil.

Another embodiment comprises cyclosporin A, Atlox 1045A®, and Cottonseed oil.

Another embodiment comprises cyclosporin A, Canarcel 20®, and Cottonseed oil.

Another embodiment comprises cyclosporin A, Canarcel 60®, and Cottonseed oil.

Another embodiment comprises cyclosporin A, Canarcel 80®, and Cottonseed oil.

Another embodiment comprises cyclosporin A, Canarcel TW 20®, and Cottonseed oil.

Another embodiment comprises cyclosporin A, Canarcel TW 60®, and Cottonseed oil.

Another embodiment comprises cyclosporin A, Canarcel TW 80®, and Cottonseed oil.

Another embodiment comprises cyclosporin A, Coladet BSB-P®, and Cottonseed oil.

Another embodiment comprises cyclosporin A, Customulse O-20®, and Cottonseed oil.

Another embodiment comprises cyclosporin A, Dehymuls E®, and Cottonseed oil.

Another embodiment comprises cyclosporin A, DeSotan SMO®, and Cottonseed oil.

Another embodiment comprises cyclosporin A, DeSotan SMO-20®, and Cottonseed oil.

Another embodiment comprises cyclosporin A, DeSotan SMT®, and Cottonseed oil.

Another embodiment comprises cyclosporin A, DeSotan SMT-20®, and Cottonseed oil.

Another embodiment comprises cyclosporin A, Durfax 60®, and Cottonseed oil.

Another embodiment comprises cyclosporin A, Durfax 65®, and Cottonseed oil.

Another embodiment comprises cyclosporin A, Durax 80®, and Cottonseed oil.

Another embodiment comprises cyclosporin A, Durtan 60®, and Cottonseed oil.

Another embodiment comprises cyclosporin A, Durtan 65®, and Cottonseed oil.

Another embodiment comprises cyclosporin A, Liposorb L®, and Cottonseed oil.

Another embodiment comprises cyclosporin A, Liposorb L-10®, and Cottonseed oil.

Another embodiment comprises cyclosporin A, Liposorb L-20®, and Cottonseed oil.

Another embodiment comprises cyclosporin A, Liposorb O®, and Cottonseed oil.

Another embodiment comprises cyclosporin A, Liposorb O-20®, and Cottonseed oil.

Another embodiment comprises cyclosporin A, Liposorb P®, and Cottonseed oil.

Another embodiment comprises cyclosporin A, Liposorb P-20®, and Cottonseed oil.

Another embodiment comprises cyclosporin A, Liposorb S®, and Cottonseed oil.

Another embodiment comprises cyclosporin A, Liposorb S-20®, and Cottonseed oil.

Another embodiment comprises cyclosporin A, Liposorb SQO®, and Cottonseed oil.

Another embodiment comprises cyclosporin A, Liposorb TO®, and Cottonseed oil.

Another embodiment comprises cyclosporin A, Liposorb TS®, and Cottonseed oil.

Another embodiment comprises cyclosporin A, Liposorb TS-20®, and Cottonseed oil.

Another embodiment comprises cyclosporin A, Lumisorb PS®, and Cottonseed oil.:

Another embodiment comprises cyclosporin A, Lumisorb SMO (T)®, and Cottonseed oil.

Another embodiment comprises cyclosporin A, Lumisorb SMS K®, and Cottonseed oil.

Another embodiment comprises cyclosporin A, Lumisorb SSO®, and Cottonseed oil.

Another embodiment comprises cyclosporin A, Lumisorb STS K®, and Cottonseed oil.

Another embodiment comprises cyclosporin A, Lumisorb STT®, and Cottonseed oil.

Another embodiment comprises cyclosporin A, Magrabar SMO®, and Cottonseed oil.

Another embodiment comprises cyclosporin A, Magrabar SMO-VEG®, and Cottonseed oil.

Another embodiment comprises cyclosporin A, Magrabar SMT®, and Cottonseed oil.

Another embodiment comprises cyclosporin A, Magrabar STO®, and Cottonseed oil.

Another embodiment comprises cyclosporin A, Miracare BC-27®, and Cottonseed oil.

Another embodiment comprises cyclosporin A, Ritabate 20®, and Cottonseed oil.

Another embodiment comprises cyclosporin A, Ritabate 40®, and Cottonseed oil.

Another embodiment comprises cyclosporin A, Ritabate 60®, and Cottonseed oil.

Another embodiment comprises cyclosporin A, Ritabate 80®, and Cottonseed oil.

Another embodiment comprises cyclosporin A, T-Maz®, and Cottonseed oil.

Another embodiment comprises cyclosporin A, Tego SML®, and Cottonseed oil.

Another embodiment comprises cyclosporin A, Tego SML 20®, and Cottonseed oil.

Another embodiment comprises cyclosporin A, Tego SMO 80 V®, and Cottonseed oil.

Another embodiment comprises cyclosporin A, Tego SMO V®, and Cottonseed oil.

Another embodiment comprises cyclosporin A, Tego SMS®, and Cottonseed oil.

Another embodiment comprises cyclosporin A, Tego STO V®, and Cottonseed oil.

Another embodiment comprises cyclosporin A, Tween 21®, and Cottonseed oil.

Another embodiment comprises cyclosporin A, Tween 40®, and Cottonseed oil.

Another embodiment comprises cyclosporin A, Tween 60®, and Cottonseed oil.

Another embodiment comprises cyclosporin A, Tween 60 K®, and Cottonseed oil.

Another embodiment comprises cyclosporin A, Tween 61®, and Cottonseed oil.

Another embodiment comprises cyclosporin A, Tween 65®, and Cottonseed oil.

Another embodiment comprises cyclosporin A, Tween 80®, and Cottonseed oil.

Another embodiment comprises cyclosporin A, Tween 80 K®, and Cottonseed oil.

Another embodiment comprises cyclosporin A, Tween 81®, and Cottonseed oil.

Another embodiment comprises cyclosporin A, Tween 85®, and Cottonseed oil.

Another embodiment comprises cyclosporin A, a Sucrose or Glucose Ester, or Derivative thereof, and Cottonseed oil.

Another embodiment comprises cyclosporin A, DeSulf GOS-P-60WCG®, and Cottonseed oil.

Another embodiment comprises cyclosporin A, Glucam E-20 Distearate®, and Cottonseed oil.

Another embodiment comprises cyclosporin A, Glucamate DOE-120®, and Cottonseed oil.

Another embodiment comprises cyclosporin A, Glucamate SSE-20®, and Cottonseed oil.

Another embodiment comprises cyclosporin A, Glucate DO®, and Cottonseed oil.

Another embodiment comprises cyclosporin A, Glucate SS®, and Cottonseed oil.

Another embodiment comprises cyclosporin A, Glucopon 425 UP®, and Cottonseed oil.

Another embodiment comprises cyclosporin A, Isolan IS®, and Cottonseed oil.

Another embodiment comprises cyclosporin A, Mazon 40®, and Cottonseed oil.

Another embodiment comprises cyclosporin A, Montanov 82®, and Cottonseed oil.

Another embodiment comprises cyclosporin A, Montanov 202®, and Cottonseed oil.

Another embodiment comprises cyclosporin A, Montanov S®, and Cottonseed oil.

Another embodiment comprises cyclosporin A, Rheozan®, and Cottonseed oil.

Another embodiment comprises cyclosporin A, Simulsol AS 48®, and Cottonseed oil.

Another embodiment comprises cyclosporin A, Simulsol SL 4®, and Cottonseed oil.

Another embodiment comprises cyclosporin A, Simulsol SL 10®, and Cottonseed oil.

Another embodiment comprises cyclosporin A, Simulsol SL 11W®, and Cottonseed oil.

Another embodiment comprises cyclosporin A, Simulsol SL 55®, and Cottonseed oil.

Another embodiment comprises cyclosporin A, Suga Nate 100 and 160®, and Cottonseed oil.

Another embodiment comprises cyclosporin A, Tego Care 450®, and Cottonseed oil.

Another embodiment comprises cyclosporin A, Tego Care CG 90®, and Cottonseed oil.

Another embodiment comprises cyclosporin A, Tego Care PS®, and Cottonseed oil.

Another embodiment comprises cyclosporin A, Tegosoft PSE 141 G®, and Cottonseed oil.

Another embodiment comprises cyclosporin A, Tegotens G 826®, and Cottonseed oil.

Another embodiment comprises cyclosporin A, Triton BG-10 (70%)®, and Cottonseed oil.

Another embodiment comprises cyclosporin A, Triton CG-110 (60%)®, and Cottonseed oil.

Another embodiment comprises cyclosporin A, Wickenol 545®, and Cottonseed oil.

Another embodiment comprises cyclosporin A, an alcohol, and Safflower oil.

Another embodiment comprises cyclosporin A, Diglycerol®, and Safflower oil.

Another embodiment comprises cyclosporin A, Hetoxide GT-80®, and Safflower oil.

Another embodiment comprises cyclosporin A, Lexemul BEO®, and Safflower oil.

Another embodiment comprises cyclosporin A, Polyglycerol-3®, and Safflower oil.

Another embodiment comprises cyclosporin A, Redicote E Series®, and Safflower oil.

Another embodiment comprises cyclosporin A, Simulsol OX 1005L®, and Safflower oil.

Another embodiment comprises cyclosporin A, Stanfax 567®, and Safflower oil.

Another embodiment comprises cyclosporin A, TA-1618®, and Safflower oil.

Another embodiment comprises cyclosporin A, Witconol H-31A®, and Safflower oil.

Another embodiment comprises cyclosporin A, an amine oxide, and Safflower oil.

Another embodiment comprises cyclosporin A, AO-405®, and Safflower oil.

Another embodiment comprises cyclosporin A, AO-455®, and Safflower oil.

Another embodiment comprises cyclosporin A, AO 728 Special®, and Safflower oil.

Another embodiment comprises cyclosporin A, Barlox 12®, and Safflower oil.

Another embodiment comprises cyclosporin A, Barlox 14®, and Safflower oil.

Another embodiment comprises cyclosporin A, Burcoxide Lo®, and Safflower oil.

Another embodiment comprises cyclosporin A, Caloxamine LO®, and Safflower oil.

Another embodiment comprises cyclosporin A, Chemoxide CAW®, and Safflower oil.

Another embodiment comprises cyclosporin A, Chemoxide LM-30®, and Safflower oil.

Another embodiment comprises cyclosporin A, Chemoxide LO®, and Safflower oil.

Another embodiment comprises cyclosporin A, Chemoxide MO®, and Safflower oil.

Another embodiment comprises cyclosporin A, Colalux CAO-35®, and Safflower oil.

Another embodiment comprises cyclosporin A, Colalux LO®, and Safflower oil.

Another embodiment comprises cyclosporin A, DeMox CAPO®, and Safflower oil.

Another embodiment comprises cyclosporin A, DeMox CSG-30®, and Safflower oil.

Another embodiment comprises cyclosporin A, DeMox LAO®, and Safflower oil.

Another embodiment comprises cyclosporin A, Emcol L®, and Safflower oil.

Another embodiment comprises cyclosporin A, Empigen OB®, and Safflower oil.

Another embodiment comprises cyclosporin A, Empigen OS/A®, and Safflower oil.

Another embodiment comprises cyclosporin A, Foamox CDO®, and Safflower oil.

Another embodiment comprises cyclosporin A, Foamox DMM®, and Safflower oil.

Another embodiment comprises cyclosporin A, Foamox DMS®, and Safflower oil.

Another embodiment comprises cyclosporin A, Genaminox KC®, and Safflower oil.

Another embodiment comprises cyclosporin A, Genaminox LA®, and Safflower oil.

Another embodiment comprises cyclosporin A, Hartofoam SAO®, and Safflower oil.

Another embodiment comprises cyclosporin A, Hartox DMCD®, and Safflower oil.

Another embodiment comprises cyclosporin A, Lipowax DAT®, and Safflower oil.

Another embodiment comprises cyclosporin A, Lipowax PB Pastilles®, and Safflower oil.

Another embodiment comprises cyclosporin A, Mackamine C8®, and Safflower oil.

Another embodiment comprises cyclosporin A, Mackamine C10®, and Safflower oil.

Another embodiment comprises cyclosporin A, Mackamine C14®, and Safflower oil.

Another embodiment comprises cyclosporin A, Mackamine CAO®, and Safflower oil.

Another embodiment comprises cyclosporin A, Mackamine CO®, and Safflower oil.

Another embodiment comprises cyclosporin A, Mackamine LO®, and Safflower oil.

Another embodiment comprises cyclosporin A, Mackamine O2®, and Safflower oil.

Another embodiment comprises cyclosporin A, Mackamine SAO®, and Safflower oil.

Another embodiment comprises cyclosporin A, Mackamine SO®, and Safflower oil.

Another embodiment comprises cyclosporin A, Mazox KCAO®, and Safflower oil.

Another embodiment comprises cyclosporin A, Monalac MO®, and Safflower oil.

Another embodiment comprises cyclosporin A, Norox LDA®, and Safflower oil.

Another embodiment comprises cyclosporin A, Rhodamox LO®, and Safflower oil.

Another embodiment comprises cyclosporin A, Schercamox C-AA®, and Safflower oil.

Another embodiment comprises cyclosporin A, Schercamox DMA®, and Safflower oil.

Another embodiment comprises cyclosporin A, Schercamox DML®, and Safflower oil.

Another embodiment comprises cyclosporin A, Schercamox DMM®, and Safflower oil.

Another embodiment comprises cyclosporin A, Schercamox DMS®, and Safflower oil.

Another embodiment comprises cyclosporin A, Tegotens DO®, and Safflower oil.

Another embodiment comprises cyclosporin A, Tomah AO-14-2®, and Safflower oil.

Another embodiment comprises cyclosporin A, Triaminox CDO®, and Safflower oil.

Another embodiment comprises cyclosporin A, a block polymer. and Safflower oil.

Another embodiment comprises cyclosporin A, AL 2070®, and Safflower oil.

Another embodiment comprises cyclosporin A, Antarox 17-R-2®, and Safflower oil.

Another embodiment comprises cyclosporin A, Antarox 25-R-2®, and Safflower oil.

Another embodiment comprises cyclosporin A, Antarox 31-R-1®, and Safflower oil.

Another embodiment comprises cyclosporin A, Antarox P-84®, and Safflower oil.

Another embodiment comprises cyclosporin A, Antarox P-104/H®, and Safflower oil.

Another embodiment comprises cyclosporin A, Arnox BP-Series®, and Safflower oil.

Another embodiment comprises cyclosporin A, Chemonic 435®, and Safflower oil.

Another embodiment comprises cyclosporin A, Chemonic D-25®, and Safflower oil.

Another embodiment comprises cyclosporin A, Chemdnic PL Series®, and Safflower oil.

Another embodiment comprises cyclosporin A, Ethox L-121®, and Safflower oil.

Another embodiment comprises cyclosporin A, Ethox L-122®, and Safflower oil.

Another embodiment comprises cyclosporin A, Genapol PF-10®, and Safflower oil.

Another embodiment comprises cyclosporin A, Genapol PF-20®, and Safflower oil.

Another embodiment comprises cyclosporin A, Genapol PF-40A®, and Safflower oil.

Another embodiment comprises cyclosporin A, Nonox 2-LF®, and Safflower oil.

Another embodiment comprises cyclosporin A, Simulsol NW 342®, and Safflower oil.

Another embodiment comprises cyclosporin A, T-Det BP-1®, and Safflower oil.

Another embodiment comprises cyclosporin A, T-Det XD®, and Safflower oil.

Another embodiment comprises cyclosporin A, T-Det XH®, and Safflower oil.

Another embodiment comprises cyclosporin A, Triton CF-32®, and Safflower oil.

Another embodiment comprises cyclosporin A, Witconol 171®, and Safflower oil.

Another embodiment comprises cyclosporin A, Witconol 324®, and Safflower oil.

Another embodiment comprises cyclosporin A, Witconol 324D®, and Safflower oil.

Another embodiment comprises cyclosporin A, Witconol PD-2000®, and Safflower oil.

Another embodiment comprises cyclosporin A, a Carboxylated Alcohol or Alkylphenol Ethoxylate, and Safflower oil.

Another embodiment comprises cyclosporin A, Emcol CN-6®, and Safflower oil.

Another embodiment comprises cyclosporin A, Ethcarb®, and Safflower oil.

Another embodiment comprises cyclosporin A, Gemtex WNT-Conc®, and Safflower oil.

Another embodiment comprises cyclosporin A, Incrodet TD7-C®, and Safflower oil.

Another embodiment comprises cyclosporin A, Marlinat CM 105/80®, and Safflower oil.

Another embodiment comprises cyclosporin A, Marlowet 1072®, and Safflower oil.

Another embodiment comprises cyclosporin A, Marlowet 4530®, and Safflower oil.

Another embodiment comprises cyclosporin A, Marlowet 4530 LF®, and Safflower oil.

Another embodiment comprises cyclosporin A, Marlowet 4534®, and Safflower oil.

Another embodiment comprises cyclosporin A, Marlowet 4538®, and Safflower oil.

Another embodiment comprises cyclosporin A, Marlowet 4539®, and Safflower oil.

Another embodiment comprises cyclosporin A, Marlowet 4539 LF®, and Safflower oil.

Another embodiment comprises cyclosporin A, Marlowet 4541®, and Safflower oil.

Another embodiment comprises cyclosporin A, Miranate LEC-80®, and Safflower oil.

Another embodiment comprises cyclosporin A, Sandopan B®, and Safflower oil.

Another embodiment comprises cyclosporin A, Sandopan B Modified®, and Safflower oil.

Another embodiment comprises cyclosporin A, Sandopan LS-24 Gel®, and Safflower oil.

Another embodiment comprises cyclosporin A, Surtine T-A®, and Safflower oil.

Another embodiment comprises cyclosporin A, a Carboxylic Acid/Fatty Acid, and Safflower oil.

Another embodiment comprises cyclosporin A, Colaterge RAM®, and Safflower oil.

Another embodiment comprises cyclosporin A, Colatrope INC®, and Safflower oil.

Another embodiment comprises cyclosporin A, Crodacid B®, and Safflower oil.

Another embodiment comprises cyclosporin A, DeTrope CA-100®, and Safflower oil.

Another embodiment comprises cyclosporin A, Latol MTO®, and Safflower oil.

Another embodiment comprises cyclosporin A, Lumulse CC-33 K®, and Safflower oil.

Another embodiment comprises cyclosporin A, Mulls 2218®, and Safflower oil.

Another embodiment comprises cyclosporin A, OL-600®, and Safflower oil.

Another embodiment comprises cyclosporin A, OL-800®, and Safflower oil.

Another embodiment comprises cyclosporin A, R-910®, and Safflower oil.

Another embodiment comprises cyclosporin A, S-210®, and Safflower oil.

Another embodiment comprises cyclosporin A, Sandopan DTC Acid®, and Safflower oil.

Another embodiment comprises cyclosporin A, Sandopan LS 24 N®, and Safflower oil.

Another embodiment comprises cyclosporin A, Sandopan MA-18®, and Safflower oil.

Another embodiment comprises cyclosporin A, an Ethoxylated Alcohol. and Safflower oil.

Another embodiment comprises cyclosporin A, Adsee 799®, and Safflower oil.

Another embodiment comprises cyclosporin A, Adsee 799®, and Safflower oil.

Another embodiment comprises cyclosporin A, Alfonic 610-3.5®, and Safflower oil.

Another embodiment comprises cyclosporin A, Alfonic 810-2®, and Safflower oil.

Another embodiment comprises cyclosporin A, Alfonic 810-6®, and Safflower oil.

Another embodiment comprises cyclosporin A, Alfonic 1012-3®, and Safflower oil.

Another embodiment comprises cyclosporin A, Alfonic 1012-5®, and Safflower oil.

Another embodiment comprises cyclosporin A, Alfonic 1216CO-1.5®, and Safflower oil.

Another embodiment comprises cyclosporin A, Alfonic 1216CO-7®, and Safflower oil.

Another embodiment comprises cyclosporin A, Alfonic 1412-3®, and Safflower oil.

Another embodiment comprises cyclosporin A, Alfonic 1412-7®, and Safflower oil.

Another embodiment comprises cyclosporin A, Arlasolve 200®, and Safflower oil.

Another embodiment comprises cyclosporin A, Arlasolve 200 Liquid®, and Safflower oil.

Another embodiment comprises cyclosporin A, Armix 180-C®, and Safflower oil.

Another embodiment comprises cyclosporin A, Armix 183®, and Safflower oil.

Another embodiment comprises cyclosporin A, Armul 2404®, and Safflower oil.

Another embodiment comprises cyclosporin A, Armul 2404®, and Safflower oil.

Another embodiment comprises cyclosporin A, Atlas EMJ-C®, and Safflower oil.

Another embodiment comprises cyclosporin A, Atlas G-2109®, and Safflower oil.

Another embodiment comprises cyclosporin A, Atlas G-3886®, and Safflower oil.

Another embodiment comprises cyclosporin A, Atlas G-3890®, and Safflower oil.

Another embodiment comprises cyclosporin A, Bio Soft E-200®, and Safflower oil.

Another embodiment comprises cyclosporin A, Bio Soft E-300®, and Safflower oil.

Another embodiment comprises cyclosporin A, Bio Soft E-400®, and Safflower oil.

Another embodiment comprises cyclosporin A, Bio Soft EN 600®, and Safflower oil.

Another embodiment comprises cyclosporin A, Bio Soft TD-400®, and Safflower oil.

Another embodiment comprises cyclosporin A, Bio Soft TD-630®, and Safflower oil.

Another embodiment comprises cyclosporin A, Brij 30®, and Safflower oil.

Another embodiment comprises cyclosporin A, Brij 52®, and Safflower oil.

Another embodiment comprises cyclosporin A, Brij 56®, and Safflower oil.

Another embodiment comprises cyclosporin A, Brij 58®, and Safflower oil.

Another embodiment comprises cyclosporin A, Brij 72®, and Safflower oil.

Another embodiment comprises cyclosporin A, Brij 76®, and Safflower oil.

Another embodiment comprises cyclosporin A, Brij 78®, and Safflower oil.

Another embodiment comprises cyclosporin A, Brij 93®, and Safflower oil.

Another embodiment comprises cyclosporin A, Brij 97®, and Safflower oil.

Another embodiment comprises cyclosporin A, Brij 98®, and Safflower oil.

Another embodiment comprises cyclosporin A, Brij 700®, and Safflower oil.

Another embodiment comprises cyclosporin A, Brij 700 S®, and Safflower oil.

Another embodiment comprises cyclosporin A, Brij 721®, and Safflower oil.

Another embodiment comprises cyclosporin A, Brij 721 S®, and Safflower oil.

Another embodiment comprises cyclosporin A, Burcoterge CDG®, and Safflower oil.

Another embodiment comprises cyclosporin A, Canasol AT 600®, and Safflower oil.

Another embodiment comprises cyclosporin A, Canasol AT 800®, and Safflower oil.

Another embodiment comprises cyclosporin A, Canasol AT 1200®, and Safflower oil.

Another embodiment comprises cyclosporin A, Canasol BJ 35®, and Safflower oil.

Another embodiment comprises cyclosporin A, Canasol BJ 36®, and Safflower oil.

Another embodiment comprises cyclosporin A, Canasol BJ 52®, and Safflower oil.

Another embodiment comprises cyclosporin A, Canasol BJ 58®, and Safflower oil.

Another embodiment comprises cyclosporin A, Canasol BJ 72®, and Safflower oil.

Another embodiment comprises cyclosporin A, Canasol BJ 78®, and Safflower oil.

Another embodiment comprises cyclosporin A, Canasol BJ 98®, and Safflower oil.

Another embodiment comprises cyclosporin A, Canasol BJ 307®, and Safflower oil.

Another embodiment comprises cyclosporin A, Cerfak 1400®, and Safflower oil.

Another embodiment comprises cyclosporin A, Cetomacrogol 1000 BP®, and Safflower oil.

Another embodiment comprises cyclosporin A, Chemonic C-2®, and Safflower oil.

Another embodiment comprises cyclosporin A, Chemonic C-10®, and Safflower oil.

Another embodiment comprises cyclosporin A, Chemonic C-20®, and Safflower oil.

Another embodiment comprises cyclosporin A, Chemonic CT-12®, and Safflower oil.

Another embodiment comprises cyclosporin A, Chemonic CT-20®, and Safflower oil.

Another embodiment comprises cyclosporin A, Chemonic CT-30®, and Safflower oil.

Another embodiment comprises cyclosporin A, Chemonic CT-55®, and Safflower oil.

Another embodiment comprises cyclosporin A, Chemonic G-7®, and Safflower oil.

Another embodiment comprises cyclosporin A, Chemonic G-26®, and Safflower oil.

Another embodiment comprises cyclosporin A, Chemonic L-4®, and Safflower oil.

Another embodiment comprises cyclosporin A, Chemonic L-7®, and Safflower oil.

Another embodiment comprises cyclosporin A, Chemonic L-12®, and Safflower oil.

Another embodiment comprises cyclosporin A, Chemonic L-23®, and Safflower oil.

Another embodiment comprises cyclosporin A, Chemonic O-2®, and Safflower oil.

Another embodiment comprises cyclosporin A, Chemonic O-5®, and Safflower oil.

Another embodiment comprises cyclosporin A, Chemonic O-10®, and Safflower oil.

Another embodiment comprises cyclosporin A, Chemonic O-20®, and Safflower oil.

Another embodiment comprises cyclosporin A, Chemonic S-2®, and Safflower oil.

Another embodiment comprises cyclosporin A, Chemonic S-10®, and Safflower oil.

Another embodiment comprises cyclosporin A, Chemonic S-20®, and Safflower oil.

Another embodiment comprises cyclosporin A, Colamulse FE®, and Safflower oil.

Another embodiment comprises cyclosporin A, Cremophor A 20®, and Safflower oil.

Another embodiment comprises cyclosporin A, Cremophor SA 2®, and Safflower oil.

Another embodiment comprises cyclosporin A, Dehydol 100®, and Safflower oil.

Another embodiment comprises cyclosporin A, Dehydol O-4®, and Safflower oil.

Another embodiment comprises cyclosporin A, Delonic C-18®, and Safflower oil.

Another embodiment comprises cyclosporin A, DeSonic 6T®, and Safflower oil.

Another embodiment comprises cyclosporin A, DeSonic 9D®, and Safflower oil.

Another embodiment comprises cyclosporin A, DeSonic 9T®, and Safflower oil.

Another embodiment comprises cyclosporin A, DeSonic 12D®, and Safflower oil.

Another embodiment comprises cyclosporin A, DeSonic 12T®, and Safflower oil.

Another embodiment comprises cyclosporin A, DeSonic 15T®, and Safflower oil.

Another embodiment comprises cyclosporin A, DeSonic TDA-9®, and Safflower oil.

Another embodiment comprises cyclosporin A, DeThox GLG-7®, and Safflower oil.

Another embodiment comprises cyclosporin A, DeThox GLG-26®, and Safflower oil.

Another embodiment comprises cyclosporin A, DeThox LA-4®, and Safflower oil.

Another embodiment comprises cyclosporin A, DeThox LA-23®, and Safflower oil.

Another embodiment comprises cyclosporin A, DeThox SA-80®, and Safflower oil.

Another embodiment comprises cyclosporin A, Disponil O5®, and Safflower oil.

Another embodiment comprises cyclosporin A, Eccoterge EO-41B®, and Safflower oil.

Another embodiment comprises cyclosporin A, Empilan KA2.5/90®, and Safflower oil.

Another embodiment comprises cyclosporin A, Empilan KA5/90®, and Safflower oil.

Another embodiment comprises cyclosporin A, Empilan KM-20®, and Safflower oil.

Another embodiment comprises cyclosporin A, Empilan KM-50®, and Safflower oil.

Another embodiment comprises cyclosporin A, Empilan L-23®, and Safflower oil.

Another embodiment comprises cyclosporin A, Ethylan 25-3®, and Safflower oil.

Another embodiment comprises cyclosporin A, Ethylan 1204®, and Safflower oil.

Another embodiment comprises cyclosporin A, Ethylan DA-4®, and Safflower oil.

Another embodiment comprises cyclosporin A, Ethylan LA-230®, and Safflower oil.

Another embodiment comprises cyclosporin A, Ethylan SN®, and Safflower oil.

Another embodiment comprises cyclosporin A, Ethylan TD-60®, and Safflower oil.

Another embodiment comprises cyclosporin A, Ethylan TD-100®, and Safflower oil.

Another embodiment comprises cyclosporin A, Ethylan TD-1407®, and Safflower oil.

Another embodiment comprises cyclosporin A, Eumulgin B1®, and Safflower oil.

Another embodiment comprises cyclosporin A, Eumulgin B2®, and Safflower oil.

Another embodiment comprises cyclosporin A, Eumulgin B3®, and Safflower oil.

Another embodiment comprises cyclosporin A, Eumulgin O-10®, and Safflower oil.

Another embodiment comprises cyclosporin A, Flo Mo 80/20®, and Safflower oil.

Another embodiment comprises cyclosporin A, Flo Mo Low Foam®, and Safflower oil.

Another embodiment comprises cyclosporin A, Forlan C-24®, and Safflower oil.

Another embodiment comprises cyclosporin A, Genapol 1454®, and Safflower oil.

Another embodiment comprises cyclosporin A, Genapol BA-020®, and Safflower oil.

Another embodiment comprises cyclosporin A, Genapol BA-040®, and Safflower oil.

Another embodiment comprises cyclosporin A, Genapol C-100®, and Safflower oil.

Another embodiment comprises cyclosporin A, Genapol DA 060®, and Safflower oil.

Another embodiment comprises cyclosporin A, Genapol HS 020®, and Safflower oil.

Another embodiment comprises cyclosporin A, Genapol HS 200®, and Safflower oil.

Another embodiment comprises cyclosporin A, Genapol ID-040®, and Safflower oil.

Another embodiment comprises cyclosporin A, Genapol ID-060®, and Safflower oil.

Another embodiment comprises cyclosporin A, Genapol ID-090®, and Safflower oil.

Another embodiment comprises cyclosporin A, Genapol LA 010®, and Safflower oil.

Another embodiment comprises cyclosporin A, Genapol LA 020®, and Safflower oil.

Another embodiment comprises cyclosporin A, Genapol LA 030®, and Safflower oil.

Another embodiment comprises cyclosporin A, Genapol LA 040®, and Safflower oil.

Another embodiment comprises cyclosporin A, Genapol LA 050®, and Safflower oil.

Another embodiment comprises cyclosporin A, Genapol LA 060®, and Safflower oil.

Another embodiment comprises cyclosporin A, Genapol LA 070®, and Safflower oil.

Another embodiment comprises cyclosporin A, Genapol LA 070S®, and Safflower oil.

Another embodiment comprises cyclosporin A, Genapol LA 230®, and Safflower oil.

Another embodiment comprises cyclosporin A, Genapol O020®, and Safflower oil.

Another embodiment comprises cyclosporin A, Genapol O050®, and Safflower oil.

Another embodiment comprises cyclosporin A, Genapol O10®, and Safflower oil.

Another embodiment comprises cyclosporin A, Genapol O200®, and Safflower oil.

Another embodiment comprises cyclosporin A, Genapol SA 030®, and Safflower oil.

Another embodiment comprises cyclosporin A, Genapol SA 120®, and Safflower oil.

Another embodiment comprises cyclosporin A, Genapol T-020®, and Safflower oil.

Another embodiment comprises cyclosporin A, Genapol UD-030®, and Safflower oil.

Another embodiment comprises cyclosporin A, Genapol UD-050®, and Safflower oil.

Another embodiment comprises cyclosporin A, Genapol UD-070®, and Safflower oil.

Another embodiment comprises cyclosporin A, Genapol UD-079®, and Safflower oil.

Another embodiment comprises cyclosporin A, Genapol UD-080®, and Safflower oil.

Another embodiment comprises cyclosporin A, Genapol UD-110®, and Safflower oil.

Another embodiment comprises cyclosporin A, Genapol X 030®, and Safflower oil.

Another embodiment comprises cyclosporin A, Genapol X 050®, and Safflower oil.

Another embodiment comprises cyclosporin A, Genapol X 060®, and Safflower oil.

Another embodiment comprises cyclosporin A, Genapol X 070®, and Safflower oil.

Another embodiment comprises cyclosporin A, Genapol X 080®, and Safflower oil.

Another embodiment comprises cyclosporin A, Genapol X 100®, and Safflower oil.

Another embodiment comprises cyclosporin A, Genapol X159®, and Safflower oil.

Another embodiment comprises cyclosporin A, Generol 122 E5®, and Safflower oil.

Another embodiment comprises cyclosporin A, Generol 122 E25®, and Safflower oil.

Another embodiment comprises cyclosporin A, Hostacerin T-3®, and Safflower oil.

Another embodiment comprises cyclosporin A, Iconol LF 110®, and Safflower oil.

Another embodiment comprises cyclosporin A, Incropol CS-20®, and Safflower oil.

Another embodiment comprises cyclosporin A, Lexemul CS-20®, and Safflower oil.

Another embodiment comprises cyclosporin A, Liponic EG-1®, and Safflower oil.

Another embodiment comprises cyclosporin A, Lipowax D®, and Safflower oil.

Another embodiment comprises cyclosporin A, Lipowax G®, and Safflower oil.

Another embodiment comprises cyclosporin A, Lipowax NI®, and Safflower oil.

Another embodiment comprises cyclosporin A, Lipowax P®, and Safflower oil.

Another embodiment comprises cyclosporin A, Lipowax P-31®, and Safflower oil.

Another embodiment comprises cyclosporin A, Lipowax PR®, and Safflower oil.

Another embodiment comprises cyclosporin A, Lumulse CS-20®, and Safflower oil.

Another embodiment comprises cyclosporin A, Macol CSA-20®, and Safflower oil.

Another embodiment comprises cyclosporin A, Marlox B 24/50®, and Safflower oil.

Another embodiment comprises cyclosporin A, Mazawet 77®, and Safflower oil.

Another embodiment comprises cyclosporin A, Norfox 1713®, and Safflower oil.

Another embodiment comprises cyclosporin A, Norfox 2579®, and Safflower oil.

Another embodiment comprises cyclosporin A, Norfox Lo Foam®, and Safflower oil.

Another embodiment comprises cyclosporin A, Promulgen D®, and Safflower oil.

Another embodiment comprises cyclosporin A, Promulgen G®, and Safflower oil.

Another embodiment comprises cyclosporin A, Renex 30®, and Safflower oil.

Another embodiment comprises cyclosporin A, Renex 36®, and Safflower oil.

Another embodiment comprises cyclosporin A, Rhodasud A 24®, and Safflower oil.

Another embodiment comprises cyclosporin A, Rhodasurf AAE-10®, and Safflower oil.

Another embodiment comprises cyclosporin A, Rhodasurt BEH-25®, and Safflower oil.

Another embodiment comprises cyclosporin A, Rhodasurl BEH-40®, and Safflower oil.

Another embodiment comprises cyclosporin A, Rhodasurd DA 530®, and Safflower oil.

Another embodiment comprises cyclosporin A, Rhodasudf DA 630®, and Safflower oil.

Another embodiment comprises cyclosporin A, Rhodasurt DA 639®, and Safflower oil.

Another embodiment comprises cyclosporin A, Rhodasurf LAN-23®, and Safflower oil.

Another embodiment comprises cyclosporin A, Rhodasurl ON-870®, and Safflower oil.

Another embodiment comprises cyclosporin A, Rhodasudf ON-877®, and Safflower oil.

Another embodiment comprises cyclosporin A, Rhodasurl TB-970 FLK®, and Safflower oil.

Another embodiment comprises cyclosporin A, Ritacet-20®, and Safflower oil.

Another embodiment comprises cyclosporin A, Ritachol 1000®, and Safflower oil.

Another embodiment comprises cyclosporin A, Ritachol 2000®, and Safflower oil.

Another embodiment comprises cyclosporin A, Ritachol 5000®, and Safflower oil.

Another embodiment comprises cyclosporin A, Ritox 35®, and Safflower oil.

Another embodiment comprises cyclosporin A, Surfonic DA-4®, and Safflower oil.

Another embodiment comprises cyclosporin A, Surfonic DA-6®, and Safflower oil.

Another embodiment comprises cyclosporin A, Surfonic L46-7®, and Safflower oil.

Another embodiment comprises cyclosporin A, Surfonic POA®, and Safflower oil.

Another embodiment comprises cyclosporin A, Synthrapol KB®, and Safflower oil.

Another embodiment comprises cyclosporin A, Teginacid®, and Safflower oil.

Another embodiment comprises cyclosporin A, Teginacid C®, and Safflower oil.

Another embodiment comprises cyclosporin A, Tegotens EC 11®, and Safflower oil.

Another embodiment comprises cyclosporin A, Tinegal NA®, and Safflower oil.

Another embodiment comprises cyclosporin A, Tomadol 400®, and Safflower oil.

Another embodiment comprises cyclosporin A, Tomadol 600®, and Safflower oil.

Another embodiment comprises cyclosporin A, Tomadol 900®, and Safflower oil.

Another embodiment comprises cyclosporin A, Uniperol O®, and Safflower oil.

Another embodiment comprises cyclosporin A, Witconol SN Series®, and Safflower oil.

Another embodiment comprises cyclosporin A, an Ethoxylated Alkylphenol. and Safflower oil.

Another embodiment comprises cyclosporin A, Antarox LF-222®, and Safflower oil.

Another embodiment comprises cyclosporin A, Atlox 775®, and Safflower oil.

Another embodiment comprises cyclosporin A, Caloxylate N-9®, and Safflower oil.

Another embodiment comprises cyclosporin A, Canasol NF-1000®, and Safflower oil.

Another embodiment comprises cyclosporin A, Canasol NF-3000®, and Safflower oil.

Another embodiment comprises cyclosporin A, Canasol NF-3070®, and Safflower oil.

Another embodiment comprises cyclosporin A, Canasol OF 1670®, and Safflower oil.

Another embodiment comprises cyclosporin A, Canasol OF 2570®, and Safflower oil.

Another embodiment comprises cyclosporin A, Canasol OF 4070®, and Safflower oil.

Another embodiment comprises cyclosporin A, Chemax DNP-8®, and Safflower oil.

Another embodiment comprises cyclosporin A, Chemax DNP-18®, and Safflower oil.

Another embodiment comprises cyclosporin A, Chemax DNP-150/50®, and Safflower oil.

Another embodiment comprises cyclosporin A, DeSonic 1.5N®, and Safflower oil.

Another embodiment comprises cyclosporin A, DeSonic 4N®, and Safflower oil.

Another embodiment comprises cyclosporin A, DeSonic 5N®, and Safflower oil.

Another embodiment comprises cyclosporin A, DeSonic 6D®, and Safflower oil.

Another embodiment comprises cyclosporin A, DeSonic 6N®, and Safflower oil.

Another embodiment comprises cyclosporin A, DeSonic 7N®, and Safflower oil.

Another embodiment comprises cyclosporin A, DeSonic 9N®, and Safflower oil.

Another embodiment comprises cyclosporin A, DeSonic 10OD®, and Safflower oil.

Another embodiment comprises cyclosporin A, DeSonic 11N®, and Safflower oil.

Another embodiment comprises cyclosporin A, DeSonic 12N®, and Safflower oil.

Another embodiment comprises cyclosporin A, DeSonic 15N®, and Safflower oil.

Another embodiment comprises cyclosporin A, DeSonic 20N®, and Safflower oil.

Another embodiment comprises cyclosporin A, Eccoscour RC®, and Safflower oil.

Another embodiment comprises cyclosporin A, Eccoterge EO-100®, and Safflower oil.

Another embodiment comprises cyclosporin A, Emulsifier 632/90%®, and Safflower oil.

Another embodiment comprises cyclosporin A, Geronol AG-821®, and Safflower oil.

Another embodiment comprises cyclosporin A, Gradonic N-95®, and Safflower oil.

Another embodiment comprises cyclosporin A, Hetoxide NP-4®, and Safflower oil.

Another embodiment comprises cyclosporin A, Hetoxide NP-30®, and Safflower oil.

Another embodiment comprises cyclosporin A, Hostapal N-100®, and Safflower oil.

Another embodiment comprises cyclosporin A, Hostapal N-110®, and Safflower oil.

Another embodiment comprises cyclosporin A, Igepal CTA-639W®, and Safflower oil.

Another embodiment comprises cyclosporin A, Igepal DAP-9®, and Safflower oil.

Another embodiment comprises cyclosporin A, Igepal OD-410®, and Safflower oil.

Another embodiment comprises cyclosporin A, Igepal SS-837®, and Safflower oil.

Another embodiment comprises cyclosporin A, Lipocol NP-9 USP®, and Safflower oil.

Another embodiment comprises cyclosporin A, Macol DNP-10®, and Safflower oil.

Another embodiment comprises cyclosporin A, Marlophen NP 5®, and Safflower oil.

Another embodiment comprises cyclosporin A, Marlophen P 1®, and Safflower oil.

Another embodiment comprises cyclosporin A, Surfonic NB®, and Safflower oil.

Another embodiment comprises cyclosporin A, Surfonic OPB-307®, and Safflower oil.

Another embodiment comprises cyclosporin A, Surfonic OPB-407®, and Safflower oil.

Another embodiment comprises cyclosporin A, Syn Fac 334®, and Safflower oil.

Another embodiment comprises cyclosporin A, Syn Fac 8216®, and Safflower oil.

Another embodiment comprises cyclosporin A, Triton N-57®, and Safflower oil.

Another embodiment comprises cyclosporin A, Trycol 6956®, and Safflower oil.

Another embodiment comprises cyclosporin A, Trycol 6961®, and Safflower oil.

Another embodiment comprises cyclosporin A, Trycol 6964®, and Safflower oil.

Another embodiment comprises cyclosporin A, Trycol 6969®, and Safflower oil.

Another embodiment comprises cyclosporin A, Trycol 6974®, and Safflower oil.

Another embodiment comprises cyclosporin A, Witbreak DRB-127®, and Safflower oil.

Another embodiment comprises cyclosporin A, Witbreak DRB-127®, and Safflower oil.

Another embodiment comprises cyclosporin A, Witconol NP Series®, and Safflower oil.

Another embodiment comprises cyclosporin A, an Ethoxylated Aryl Phenol, and Safflower oil.

Another embodiment comprises cyclosporin A, Soprophor BSU®, and Safflower oil.

Another embodiment comprises cyclosporin A, Soprophor CY/8®, and Safflower oil.

Another embodiment comprises cyclosporin A, Soprophor S/25®, and Safflower oil.

Another embodiment comprises cyclosporin A, Witconol NIO®, and Safflower oil.

Another embodiment comprises cyclosporin A, Witconol NIW®, and Safflower oil.

Another embodiment comprises cyclosporin A, Witconol S-100®, and Safflower oil.

Another embodiment comprises cyclosporin A, an Ethoxvlated Fatty Acid, and Safflower oil.

Another embodiment comprises cyclosporin A, Aldo PGHMS®, and Safflower oil.

Another embodiment comprises cyclosporin A, Alkamuls TO-15/HR®, and Safflower oil.

Another embodiment comprises cyclosporin A, Armotan AL-69-66®, and Safflower oil.

Another embodiment comprises cyclosporin A, Cerasynt 840®, and Safflower oil.

Another embodiment comprises cyclosporin A, Cerasynt 945®, and Safflower oil.

Another embodiment comprises cyclosporin A, Crystal Inhibitor No. 5®, and Safflower oil.

Another embodiment comprises cyclosporin A, DeThox Acid L-9®, and Safflower oil.

Another embodiment comprises cyclosporin A, DeThox Acid S-8®, and Safflower oil.

Another embodiment comprises cyclosporin A, Ethofat 242/25®, and Safflower oil.

Another embodiment comprises cyclosporin A, Hydropalat 65®, and Safflower oil.

Another embodiment comprises cyclosporin A, Lipo EGMS®, and Safflower oil.

Another embodiment comprises cyclosporin A, Lipopeg 2 DL®, and Safflower oil.

Another embodiment comprises cyclosporin A, Lipopeg 4 DL®, and Safflower oil.

Another embodiment comprises cyclosporin A, Lipopeg 4-L®, and Safflower oil.

Another embodiment comprises cyclosporin A, Lipopeg 39-S®, and Safflower oil.

Another embodiment comprises cyclosporin A, Lipopeg 4-S®, and Safflower oil.

Another embodiment comprises cyclosporin A, Lipopeg 10-S®, and Safflower oil.

Another embodiment comprises cyclosporin A, Lipopeg 100-S®, and Safflower oil.

Another embodiment comprises cyclosporin A, Lipopeg 6000DS®, and Safflower oil.

Another embodiment comprises cyclosporin A, Lumulse 40-L®, and Safflower oil.

Another embodiment comprises cyclosporin A, Lumulse 40-S®, and Safflower oil.

Another embodiment comprises cyclosporin A, Lumulse 42-L®, and Safflower oil.

Another embodiment comprises cyclosporin A, Lumulse 42-S®, and Safflower oil.

Another embodiment comprises cyclosporin A, Lumulse 100-S®, and Safflower oil.

Another embodiment comprises cyclosporin A, Lumulse 602-S®, and Safflower oil.

Another embodiment comprises cyclosporin A, Magrabar PGE-20-0®, and Safflower oil.

Another embodiment comprises cyclosporin A, Magrabar PGE-20L®, and Safflower oil.

Another embodiment comprises cyclosporin A, Magrabar PGE-20T®, and Safflower oil.

Another embodiment comprises cyclosporin A, Magrabar PGE-22-0®, and Safflower oil.

Another embodiment comprises cyclosporin A, Magrabar PGE-22L®, and Safflower oil.

Another embodiment comprises cyclosporin A, Magrabar PGE-22T®, and Safflower oil.

Another embodiment comprises cyclosporin A, Magrabar PGE-40-0®, and Safflower oil.

Another embodiment comprises cyclosporin A, Magrabar PGE-40L®, and Safflower oil.

Another embodiment comprises cyclosporin A, Magrabar PGE-40T®, and Safflower oil.

Another embodiment comprises cyclosporin A, Magrabar PGE-42-0®, and Safflower oil.

Another embodiment comprises cyclosporin A, Magrabar PGE-42L®, and Safflower oil.

Another embodiment comprises cyclosporin A, Magrabar PGE-42T®, and Safflower oil.

Another embodiment comprises cyclosporin A, Magrabar PGE-60-0®, and Safflower oil.

Another embodiment comprises cyclosporin A, Magrabar PGE-60L®, and Safflower oil.

Another embodiment comprises cyclosporin A, Magrabar PGE-60T®, and Safflower oil.

Another embodiment comprises cyclosporin A, Magrabar PGE-62-0®, and Safflower oil.

Another embodiment comprises cyclosporin A, Magrabar PGE-62L®, and Safflower oil.

Another embodiment comprises cyclosporin A, Magrabar PGE-62T®, and Safflower oil.

Another embodiment comprises cyclosporin A, Mapeg S-40K®, and Safflower oil.

Another embodiment comprises cyclosporin A, Marlowet OTS®, and Safflower oil.

Another embodiment comprises cyclosporin A, Naturechem PGR®, and Safflower oil.

Another embodiment comprises cyclosporin A, PG No. 4®, and Safflower oil.

Another embodiment comprises cyclosporin A, Renex 20®, and Safflower oil.

Another embodiment comprises cyclosporin A, Ritox 52®, and Safflower oil.

Another embodiment comprises cyclosporin A, Ritox 53®, and Safflower oil.

Another embodiment comprises cyclosporin A, Ritox 59®, and Safflower oil.

Another embodiment comprises cyclosporin A, Surfax 8916/A®, and Safflower oil.

Another embodiment comprises cyclosporin A, Tego Acid S 40 P®, and Safflower oil.

Another embodiment comprises cyclosporin A, Tego Acid S 100 P®, and Safflower oil.

Another embodiment comprises cyclosporin A, Tween 20®, and Safflower oil.

Another embodiment comprises cyclosporin A, Volpo 131®, and Safflower oil.

Another embodiment comprises cyclosporin A, an Ethoxylated Fatty Ester or Oil. and Safflower oil.

Another embodiment comprises cyclosporin A, Acconon 6-C10®, and Safflower oil.

Another embodiment comprises cyclosporin A, Acconon CC-6®, and Safflower oil.

Another embodiment comprises cyclosporin A, Acconon CO-7®, and Safflower oil.

Another embodiment comprises cyclosporin A, Aldosperse 40/60 FG®, and Safflower oil.

Another embodiment comprises cyclosporin A, Aldosperse ML-23®, and Safflower oil.

Another embodiment comprises cyclosporin A, Aldosperse MS-20 FG®, and Safflower oil.

Another embodiment comprises cyclosporin A, Alkamuls EL-620®, and Safflower oil.

Another embodiment comprises cyclosporin A, Alkamuls EL-719®, and Safflower oil.

Another embodiment comprises cyclosporin A, Alkamuls EL-985®, and Safflower oil.

Another embodiment comprises cyclosporin A, Arlatone G®, and Safflower oil.

Another embodiment comprises cyclosporin A, Arlatone T®, and Safflower oil.

Another embodiment comprises cyclosporin A, Atlas G-1045A®, and Safflower oil.

Another embodiment comprises cyclosporin A, Atlas G-1086®, and Safflower oil.

Another embodiment comprises cyclosporin A, Atlas G-1087®, and Safflower oil.

Another embodiment comprises cyclosporin A, Atlas G-1089®, and Safflower oil.

Another embodiment comprises cyclosporin A, Atlas G-1096®, and Safflower oil.

Another embodiment comprises cyclosporin A, Atlas G-1292®, and Safflower oil.

Another embodiment comprises cyclosporin A, Atlas G-1293®, and Safflower oil.

Another embodiment comprises cyclosporin A, Atlas G-1300®, and Safflower oil.

Another embodiment comprises cyclosporin A, Atlas G-7076®, and Safflower oil.

Another embodiment comprises cyclosporin A, Capmul EMG®, and Safflower oil.

Another embodiment comprises cyclosporin A, Chemonic CO-40®, and Safflower oil.

Another embodiment comprises cyclosporin A, Chemonic LI-3®, and Safflower oil.

Another embodiment comprises cyclosporin A, Chemonic LI-7®, and Safflower oil.

Another embodiment comprises cyclosporin A, Cirrasol GM®, and Safflower oil.

Another embodiment comprises cyclosporin A, Cremophor CO 40®, and Safflower oil.

Another embodiment comprises cyclosporin A, Cremophor CO 410®, and Safflower oil.

Another embodiment comprises cyclosporin A, Cremophor EL®, and Safflower oil.

Another embodiment comprises cyclosporin A, Cremophor GC7®, and Safflower oil.

Another embodiment comprises cyclosporin A, Cremophor RH-40®, and Safflower oil.

Another embodiment comprises cyclosporin A, Crovol A-40®, and Safflower oil.

Another embodiment comprises cyclosporin A, Crovol A-70®, and Safflower oil.

Another embodiment comprises cyclosporin A, Crovol M-70®, and Safflower oil.

Another embodiment comprises cyclosporin A, Crovol PK-70®, and Safflower oil.

Another embodiment comprises cyclosporin A, Cutina E-24®, and Safflower oil.

Another embodiment comprises cyclosporin A, Dacospin 12-R®, and Safflower oil.

Another embodiment comprises cyclosporin A, Dehymuls HRE-7®, and Safflower oil.

Another embodiment comprises cyclosporin A, DeSonic 30C®, and Safflower oil.

Another embodiment comprises cyclosporin A, DeSonic 36C®, and Safflower oil.

Another embodiment comprises cyclosporin A, DeSonic 40C®, and Safflower oil.

Another embodiment comprises cyclosporin A, Durfax 60®, and Safflower oil.

Another embodiment comprises cyclosporin A, Durfax 65®, and Safflower oil.

Another embodiment comprises cyclosporin A, Durfax 80®, and Safflower oil.

Another embodiment comprises cyclosporin A, Durdax EOM®, and Safflower oil.

Another embodiment comprises cyclosporin A, Eccoterge NF-2®, and Safflower oil.

Another embodiment comprises cyclosporin A, Emulpon CO-360®, and Safflower oil.

Another embodiment comprises cyclosporin A, Emulpon CO-550®, and Safflower oil.

Another embodiment comprises cyclosporin A, Emulsogen EL®, and Safflower oil.

Another embodiment comprises cyclosporin A, Emulsogen HCO 040®, and Safflower oil.

Another embodiment comprises cyclosporin A, Emulsogen HCO 060®, and Safflower oil.

Another embodiment comprises cyclosporin A, Emulsynt 1055®, and Safflower oil.

Another embodiment comprises cyclosporin A, Ethox 3095®, and Safflower oil.

Another embodiment comprises cyclosporin A, Eumulgin RO-40®, and Safflower oil.

Another embodiment comprises cyclosporin A, Genapol G-260®, and Safflower oil.

Another embodiment comprises cyclosporin A, Glycosperse L-20®, and Safflower oil.

Another embodiment comprises cyclosporin A, Glycosperse O-5®, and Safflower oil.

Another embodiment comprises cyclosporin A, Glycosperse O-20®, and Safflower oil.

Another embodiment comprises cyclosporin A, Glycosperse O-20 FG®, and Safflower oil.

Another embodiment comprises cyclosporin A, Glycosperse S-20®, and Safflower oil.

Another embodiment comprises cyclosporin A, Glycosperse S-20 FG®, and Safflower oil.

Another embodiment comprises cyclosporin A, Glycosperse TS-20®, and Safflower oil.

Another embodiment comprises cyclosporin A, Glycosperse TS-20 FG®, and Safflower oil.

Another embodiment comprises cyclosporin A, Hetan SL®, and Safflower oil.

Another embodiment comprises cyclosporin A, Hetan SO®, and Safflower oil.

Another embodiment comprises cyclosporin A, Hetan SS®, and Safflower oil.

Another embodiment comprises cyclosporin A, Hetoxide C-2®, and Safflower oil.

Another embodiment comprises cyclosporin A, Hetoxide C-9®, and Safflower oil.

Another embodiment comprises cyclosporin A, Hetoxide C-15®, and Safflower oil.

Another embodiment comprises cyclosporin A, Hetoxide C-25®, and Safflower oil.

Another embodiment comprises cyclosporin A, Hetoxide C-40®, and Safflower oil.

Another embodiment comprises cyclosporin A, Hetoxide C-200®, and Safflower oil.

Another embodiment comprises cyclosporin A, Hetoxide C-200-50%®, and Safflower oil.

Another embodiment comprises cyclosporin A, Hetoxide GC-30®, and Safflower oil.

Another embodiment comprises cyclosporin A, Hetoxide HC-60®, and Safflower oil.

Another embodiment comprises cyclosporin A, Ice No. 2®, and Safflower oil.

Another embodiment comprises cyclosporin A, Incrocas 30/40®, and Safflower oil.

Another embodiment comprises cyclosporin A, Lexol EC®, and Safflower oil.

Another embodiment comprises cyclosporin A, Lexol EO®, and Safflower oil.

Another embodiment comprises cyclosporin A, Lipocol HCO-40®, and Safflower oil.

Another embodiment comprises cyclosporin A, Lipocol HCO-60®, and Safflower oil.

Another embodiment comprises cyclosporin A, Lipocol O-3 Special®, and Safflower oil.

Another embodiment comprises cyclosporin A, Lipopeg 2-L®, and Safflower oil.

Another embodiment comprises cyclosporin A, Lipopeg 4-DO®, and Safflower oil.

Another embodiment comprises cyclosporin A, Lipopeg 4-DS®, and Safflower oil.

Another embodiment comprises cyclosporin A, Lipovol GTB®, and Safflower oil.

Another embodiment comprises cyclosporin A, Lonzest SML-20®, and Safflower oil.

Another embodiment comprises cyclosporin A, Lonzest SMO-20®, and Safflower oil.

Another embodiment comprises cyclosporin A, Lonzest SMS-20®, and Safflower oil.

Another embodiment comprises cyclosporin A, Lonzest STO-20®, and Safflower oil.

Another embodiment comprises cyclosporin A, Lonzest STS-20®, and Safflower oil.

Another embodiment comprises cyclosporin A, Lumulse GR-40®, and Safflower oil.

Another embodiment comprises cyclosporin A, Lumulse GRH-40®, and Safflower oil.

Another embodiment comprises cyclosporin A, Lumulse POE (7) GML®, and Safflower oil.

Another embodiment comprises cyclosporin A, Lumulse POE (12) Glyc®, and Safflower oil.

Another embodiment comprises cyclosporin A, Lumulse POE (40) MS KP®, and Safflower oil.

Another embodiment comprises cyclosporin A, Marlowet 4750®, and Safflower oil.

Another embodiment comprises cyclosporin A, Marlowet LVS®, and Safflower oil.

Another embodiment comprises cyclosporin A, Marlowet R 11®, and Safflower oil.

Another embodiment comprises cyclosporin A, Marlowet R 40®, and Safflower oil.

Another embodiment comprises cyclosporin A, Mazol 80 MGK®, and Safflower oil.

Another embodiment comprises cyclosporin A, Nonionic Emulsifier T-9®, and Safflower oil.

Another embodiment comprises cyclosporin A, Oronal LCG®, and Safflower oil.

Another embodiment comprises cyclosporin A, Polyderm PPI-CO-200®, and Safflower oil.

Another embodiment comprises cyclosporin A, Polyderm PPI-CO-40®, and Safflower oil.

Another embodiment comprises cyclosporin A, Rewoderm LI 520-70®, and Safflower oil.

Another embodiment comprises cyclosporin A, Ritapeg 150 DS®, and Safflower oil.

Another embodiment comprises cyclosporin A, Sottigen 767®, and Safflower oil.

Another embodiment comprises cyclosporin A, Surtactol 318®, and Safflower oil.

Another embodiment comprises cyclosporin A, Surfactol 365®, and Safflower oil.

Another embodiment comprises cyclosporin A, Syn Lube 107®, and Safflower oil.

Another embodiment comprises cyclosporin A, Syn Lube 728®, and Safflower oil.

Another embodiment comprises cyclosporin A, Syn Lube 1632H®, and Safflower oil.

Another embodiment comprises cyclosporin A, Syn Lube 6277-A®, and Safflower oil.

Another embodiment comprises cyclosporin A, T-Det C-20®, and Safflower oil.

Another embodiment comprises cyclosporin A, T-Det C-40®, and Safflower oil.

Another embodiment comprises cyclosporin A, Tally 100 Plus®, and Safflower oil.

Another embodiment comprises cyclosporin A, Uniperol EL®, and Safflower oil.

Another embodiment comprises cyclosporin A, a Fatty Ester, and Safflower oil.

Another embodiment comprises cyclosporin A, Actralube-Syn 147®, and Safflower oil.

Another embodiment comprises cyclosporin A, Atlas G-1556®, and Safflower oil.

Another embodiment comprises cyclosporin A, Atlas G-1564®, and Safflower oil.

Another embodiment comprises cyclosporin A, Atlasol Base Oil S®, and Safflower oil.

Another embodiment comprises cyclosporin A, Base ML®, and Safflower oil.

Another embodiment comprises cyclosporin A, Base MT®, and Safflower oil.

Another embodiment comprises cyclosporin A, Cerasynt 303®, and Safflower oil.

Another embodiment comprises cyclosporin A, Dermol 1012®, and Safflower oil.

Another embodiment comprises cyclosporin A, Kemester 4000®, and Safflower oil.

Another embodiment comprises cyclosporin A, Lactipol S®, and Safflower oil.

Another embodiment comprises cyclosporin A, Magrabar PGO®, and Safflower oil.

Another embodiment comprises cyclosporin A, Mayco Base BFO®, and Safflower oil.

Another embodiment comprises cyclosporin A, Methyl Linoleate®, and Safflower oil.

Another embodiment comprises cyclosporin A, Pationic 122A®, and Safflower oil.

Another embodiment comprises cyclosporin A, Pationic 138C®, and Safflower oil.

Another embodiment comprises cyclosporin A, Pationic CSL®, and Safflower oil.

Another embodiment comprises cyclosporin A, Pationic ISL®, and Safflower oil.

Another embodiment comprises cyclosporin A, Pationic SBL®, and Safflower oil.

Another embodiment comprises cyclosporin A, Pationic SSL®, and Safflower oil.

Another embodiment comprises cyclosporin A, Ritasol®, and Safflower oil.

Another embodiment comprises cyclosporin A, Tego Alkanol CS 20®, and Safflower oil.

Another embodiment comprises cyclosporin A, Tego Alkanol L23P®, and Safflower oil.

Another embodiment comprises cyclosporin A, Tego Alkanol S2®, and Safflower oil.

Another embodiment comprises cyclosporin A, Tego Alkanol S20 P®, and Safflower oil.

Another embodiment comprises cyclosporin A, Triemulsifier 600 MS®, and Safflower oil.

Another embodiment comprises cyclosporin A, a Glycerol Esters. and Safflower oil.

Another embodiment comprises cyclosporin A, Agro #9 Wint SBO®, and Safflower oil.

Another embodiment comprises cyclosporin A, Ahcovel Base 700®, and Safflower oil.

Another embodiment comprises cyclosporin A, Aldo HMS FG®, and Safflower oil.

Another embodiment comprises cyclosporin A, Aldo MLD®, and Safflower oil.

Another embodiment comprises cyclosporin A, Aldo MLD FG®, and Safflower oil.

Another embodiment comprises cyclosporin A, Aldo MO FG®, and Safflower oil.

Another embodiment comprises cyclosporin A, Aldo MS®, and Safflower oil.

Another embodiment comprises cyclosporin A, Aldo MS FG®, and Safflower oil.

Another embodiment comprises cyclosporin A, Aldo MS LG FG®, and Safflower oil.

Another embodiment comprises cyclosporin A, Aldo MSD®, and Safflower oil.

Another embodiment comprises cyclosporin A, Aldo MSD FG®, and Safflower oil.

Another embodiment comprises cyclosporin A, Aldosperse O-20 FG®, and Safflower oil.

Another embodiment comprises cyclosporin A, Aldosperse TS-20 FG®, and Safflower oil.

Another embodiment comprises cyclosporin A, Aldosperse TS-40 FG®, and Safflower oil.

Another embodiment comprises cyclosporin A, Arlacel 165®, and Safflower oil.

Another embodiment comprises cyclosporin A, Arlacel 186®, and Safflower oil.

Another embodiment comprises cyclosporin A, Capmul GMO®, and Safflower oil.

Another embodiment comprises cyclosporin A, Capmul GMS®, and Safflower oil.

Another embodiment comprises cyclosporin A, Caprol 3GO®, and Safflower oil.

Another embodiment comprises cyclosporin A, Caprol 3GVS®, and Safflower oil.

Another embodiment comprises cyclosporin A, Caprol 6G2S®, and Safflower oil.

Another embodiment comprises cyclosporin A, Caprol 10G40®, and Safflower oil.

Another embodiment comprises cyclosporin A, Caprol 10 G100®, and Safflower oil.

Another embodiment comprises cyclosporin A, Caprol ET®, and Safflower oil.

Another embodiment comprises cyclosporin A, Caprol PGE860®, and Safflower oil.

Another embodiment comprises cyclosporin A, Cerasynt 945®, and Safflower oil.

Another embodiment comprises cyclosporin A, Cerasynt GMS®, and Safflower oil.

Another embodiment comprises cyclosporin A, Cerasynt Q®, and Safflower oil.

Another embodiment comprises cyclosporin A, Cerasynt SD®, and Safflower oil.

Another embodiment comprises cyclosporin A, Cerasynt WM®, and Safflower oil.

Another embodiment comprises cyclosporin A, Chemsperse 14®, and Safflower oil.

Another embodiment comprises cyclosporin A, Cremophor GO-32®, and Safflower oil.

Another embodiment comprises cyclosporin A, Cremophor GS11®, and Safflower oil.

Another embodiment comprises cyclosporin A, Cremophor GS-32®, and Safflower oil.

Another embodiment comprises cyclosporin A, Cutina KD-16®, and Safflower oil.

Another embodiment comprises cyclosporin A, Dehymuls PGPH®, and Safflower oil.

Another embodiment comprises cyclosporin A, Dermol DGDIS®, and Safflower oil.

Another embodiment comprises cyclosporin A, Dermol DGMIS®, and Safflower oil.

Another embodiment comprises cyclosporin A, Dermol G-76®, and Safflower oil.

Another embodirment comprises cyclosporin A, Dermol G-7DI®, and Safflower oil.

Another embodiment comprises cyclosporin A, Dermol NGDI®, and Safflower oil.

Another embodiment comprises cyclosporin A, Dermolan GLH®, and Safflower oil.

Another embodiment comprises cyclosporin A, Drewmulse GMO®, and Safflower oil.

Another embodiment comprises cyclosporin A, Drewpol 3-5-M®, and Safflower oil.

Another embodiment comprises cyclosporin A, Durlac 100 W®, and Safflower oil.

Another embodiment comprises cyclosporin A, Dur-Lo®, and Safflower oil.

Another embodiment comprises cyclosporin A, Dynasan 118®, and Safflower oil.

Another embodiment comprises cyclosporin A, EC-25®, and Safflower oil.

Another embodiment comprises cyclosporin A, EM 40®, and Safflower oil.

Another embodiment comprises cyclosporin A, Emerest 2400®, and Safflower oil.

Another embodiment comprises cyclosporin A, Emerest 2452®, and Safflower oil.

Another embodiment comprises cyclosporin A, Empilan G-26®, and Safflower oil.

Another embodiment comprises cyclosporin A, Genapol TSM®, and Safflower oil.

Another embodiment comprises cyclosporin A, Hostacerin DGI®, and Safflower oil.

Another embodiment comprises cyclosporin A, Hostacerin DGL®, and Safflower oil.

Another embodiment comprises cyclosporin A, Hostacerin DGMS®, and Safflower oil.

Another embodiment comprises cyclosporin A, Hostacerin DGSB®, and Safflower oil.

Another embodiment comprises cyclosporin A, Ice No. 2®, and Safflower oil.

Another embodiment comprises cyclosporin A, Imwitor 742®, and Safflower oil.

Another embodiment comprises cyclosporin A, lmwitor 780 K®, and Safflower oil.

Another embodiment comprises cyclosporin A, lmwitor 960 Flakes®, and Safflower oil.

Another embodiment comprises cyclosporin A, Isolan GI 34®, and Safflower oil.

Another embodiment comprises cyclosporin A, Isolan GO 33®, and Safflower oil.

Another embodiment comprises cyclosporin A, Kemester 1000®, and Safflower oil.

Another embodiment comprises cyclosporin A, Kemester 2000®, and Safflower oil.

Another embodiment comprises cyclosporin A, Kemester 2000®, and Safflower oil.

Another embodiment comprises cyclosporin A, Kemester 6000SE®, and Safflower oil.

Another embodiment comprises cyclosporin A, Lamecreme DGE 18®, and Safflower oil.

Another embodiment comprises cyclosporin A, Lexemul 515®, and Safflower oil.

Another embodiment comprises cyclosporin A, Lexemul 561®, and Safflower oil.

Another embodiment comprises cyclosporin A, Lexemul APR®, and Safflower oil.

Another embodiment comprises cyclosporin A, Lexemul AS®, and Safflower oil.

Another embodiment comprises cyclosporin A, Lexemul GDL®, and Safflower oil.

Another embodiment comprises cyclosporin A, Lexemul T®, and Safflower oil.

Another embodiment comprises cyclosporin A, Lipomulse 165®, and Safflower oil.

Another embodiment comprises cyclosporin A, Lumulse GML K®, and Safflower oil.

Another embodiment comprises cyclosporin A, Lumulse GMO K®, and Safflower oil.

Another embodiment comprises cyclosporin A, Lumulse GMR K®, and Safflower oil.

Another embodiment comprises cyclosporin A, Lumulse GMT K®, and Safflower oil.

Another embodiment comprises cyclosporin A, Magrabar GMC®, and Safflower oil.

Another embodiment comprises cyclosporin A, Magrabar GMO-CK®, and Safflower oil.

Another embodiment comprises cyclosporin A, Magrabar MDG-5050®, and Safflower oil.

Another embodiment comprises cyclosporin A, Magrabar PGO-315®, and Safflower oil.

Another embodiment comprises cyclosporin A, Magrabar PGO-1010®, and Safflower oil.

Another embodiment comprises cyclosporin A, Mazol 300K®, and Safflower oil.

Another embodiment comprises cyclosporin A, Mazol GMO-K®, and Safflower oil.

Another embodiment comprises cyclosporin A, Mazol GMS-K®, and Safflower oil.

Another embodiment comprises cyclosporin A, Mazol PG031-K®, and Safflower oil.

Another embodiment comprises cyclosporin A, Miglyol 812®, and Safflower oil.

Another embodiment comprises cyclosporin A, Norlox 165C®, and Safflower oil.

Another embodiment comprises cyclosporin A, Schercemol GMIS®, and Safflower oil.

Another embodiment comprises cyclosporin A, Tegin®, and Safflower oil.

Another embodiment comprises cyclosporin A, Tegin 4100 Pellets®, and Safflower oil.

Another embodiment comprises cyclosporin A, Tegin M Pellets®, and Safflower oil.

Another embodiment comprises cyclosporin A, Tegin OV®, and Safflower oil.

Another embodiment comprises cyclosporin A, Teginacid H®, and Safflower oil.

Another embodiment comprises cyclosporin A, Tego Cosmo P813®, and Safflower oil.

Another embodiment comprises cyclosporin A, Wickenol 535®, and Safflower oil.

Another embodiment comprises cyclosporin A, Witconol 14®, and Safflower oil.

Another embodiment comprises cyclosporin A, Witconol 14®, and Safflower oil.

Another embodiment comprises cyclosporin A, Witconol 14F®, and Safflower oil.

Another embodiment comprises cyclosporin A, Witconol 18L®, and Safflower oil.

Another embodiment comprises cyclosporin A, Witconol GOT®, and Safflower oil.

Another embodiment comprises cyclosporin A, Witconol MST®, and Safflower oil.

Another embodiment comprises cyclosporin A, Witconol RHT®, and Safflower oil.

Another embodiment comprises cyclosporin A, a Glycol Ester. and Safflower oil.

Another embodiment comprises cyclosporin A, Alkamuls 600 DO®, and Safflower oil.

Another embodiment comprises cyclosporin A, Alkamuls SEG®, and Safflower oil.

Another embodiment comprises cyclosporin A, Atlas EM-2®, and Safflower oil.

Another embodiment comprises cyclosporin A, Cerasynt IP®, and Safflower oil.

Another embodiment comprises cyclosporin A, Cerasynt M®, and Safflower oil.

Another embodiment comprises cyclosporin A, Cerasynt MN®, and Safflower oil.

Another embodiment comprises cyclosporin A, Cerasynt PA®, and Safflower oil.

Another embodiment comprises cyclosporin A, Chemsperse EGDS®, and Safflower oil.

Another embodiment comprises cyclosporin A, Chemsperse EGMS®, and Safflower oil.

Another embodiment comprises cyclosporin A, Colonial Monolaurin®, and Safflower oil.

Another embodiment comprises cyclosporin A, DeMuls SGE-95®, and Safflower oil.

Another embodiment comprises cyclosporin A, Eccoterge 200®, and Safflower oil.

Another embodiment comprises cyclosporin A, Emerest 2380®, and Safflower oil.

Another embodiment comprises cyclosporin A, Ethox 2610®, and Safflower oil.

Another embodiment comprises cyclosporin A, Ethox DO-9®, and Safflower oil.

Another embodiment comprises cyclosporin A, Ethox DO-14®, and Safflower oil.

Another embodiment comprises cyclosporin A, Ethox SO-9®, and Safflower oil.

Another embodiment comprises cyclosporin A, Fizul MD-318®, and Safflower oil.

Another embodiment comprises cyclosporin A, Genapol EGDS-VHP®, and Safflower oil.

Another embodiment comprises cyclosporin A, Genapol TS Powder®, and Safflower oil.

Another embodiment comprises cyclosporin A, Hostacerin WO®, and Safflower oil.

Another embodiment comprises cyclosporin A, Inversol 140®, and Safflower oil.

Another embodiment comprises cyclosporin A, Kemester 104®, and Safflower oil.

Another embodiment comprises cyclosporin A, Kemester 205®, and Safflower oil.

Another embodiment comprises cyclosporin A, Kemester 226®, and Safflower oil.

Another embodiment comprises-cyclosporin A, Kemester 5221SE®, and Safflower oil.

Another embodiment comprises cyclosporin A, Kemester EGDS®, and Safflower oil.

Another embodiment comprises cyclosporin A, Lexemul EGDS®, and Safflower oil.

Another embodiment comprises cyclosporin A, Lexemul EGMS®, and Safflower oil.

Another embodiment comprises cyclosporin A, Lexemul P®, and Safflower oil.

Another embodiment comprises cyclosporin A, Lipo DGLS®, and Safflower oil.

Another embodiment comprises cyclosporin A, Lipo EGDS®, and Safflower oil.

Another embodiment comprises cyclosporin A, Lipo PGMS®, and Safflower oil.

Another embodiment comprises cyclosporin A, Liposorb S-4®, and Safflower oil.

Another embodiment comprises cyclosporin A, Liposorb TO-20®, and Safflower oil.

Another embodiment comprises cyclosporin A, Lumulse PGO®, and Safflower oil.

Another embodiment comprises cyclosporin A, Mackester EGDS®, and Safflower oil.

Another embodiment comprises cyclosporin A, Mackester EGMS®, and Safflower oil.

Another embodiment comprises cyclosporin A, Mackester GSTP®, and Safflower oil.

Another embodiment comprises cyclosporin A, Mackester Series®, and Safflower oil.

Another embodiment comprises cyclosporin A, Magrabar PDG-50®, and Safflower oil.

Another embodiment comprises cyclosporin A, Mapeg 6000 DS®, and Safflower oil.

Another embodiment comprises cyclosporin A, Marlowet 4702®, and Safflower oil.

Another embodiment comprises cyclosporin A, Monalube 305®, and Safflower oil.

Another embodiment comprises cyclosporin A, Monalube 310®, and Safflower oil.

Another embodiment comprises cyclosporin A, Monalube 315®, and Safflower oil.

Another embodiment comprises cyclosporin A, Monalube 320®, and Safflower oil.

Another embodiment comprises cyclosporin A, Monalube 325®, and Safflower oil.

Another embodiment comprises cyclosporin A, Monalube 330®, and Safflower oil.

Another embodiment comprises cyclosporin A, Naturechem PGHS®, and Safflower oil.

Another embodiment comprises cyclosporin A, Polycastorol PLO-840®, and Safflower oil.

Another embodiment comprises cyclosporin A, Polytex 10OM®, and Safflower oil.

Another embodiment comprises cyclosporin A, Ritasynt IP®, and Safflower oil.

Another embodiment comprises cyclosporin A, Ross Chem PEG 600 DT®, and Safflower oil.

Another embodiment comprises cyclosporin A, Schercemol PGMS®, and Safflower oil.

Another embodiment comprises cyclosporin A, Sponto H-44C®, and Safflower oil.

Another embodiment comprises cyclosporin A, Tegin G®, and Safflower oil.

Another embodiment comprises cyclosporin A, Witbreak DGE-182®, and Safflower oil.

Another embodiment comprises cyclosporin A, Witbreak DGE-182®, and Safflower oil.

Another embodiment comprises cyclosporin A, Witbreak DRA-21®, and Safflower oil.

Another embodiment comprises cyclosporin A, Witbreak DRA-21®, and Safflower oil.

Another embodiment comprises cyclosporin A, Witbreak DRA-50®, and Safflower oil.

Another embodiment comprises cyclosporin A, Witbreak DRA-50®, and Safflower oil.

Another embodiment comprises cyclosporin A, Witconol F26-46®, and Safflower oil.

Another embodiment comprises cyclosporin A, Witconol H-32®, and Safflower oil.

Another embodiment comprises cyclosporin A, Witconol H-33®, and Safflower oil.

Another embodiment comprises cyclosporin A, Witconol H-35A®, and Safflower oil.

Another embodiment comprises cyclosporin A, Witconol RHP®, and Safflower oil.

Another embodiment comprises cyclosporin A, a Lanolin-based Derivative, and Safflower oil.

Another embodiment comprises cyclosporin A, Amerchol CAB®, and Safflower oil.

Another embodiment comprises cyclosporin A, Amerchol L-101®, and Safflower oil.

Another embodiment comprises cyclosporin A, Amerlate LFA-LO®, and Safflower oil.

Another embodiment comprises cyclosporin A, Amerlate P®, and Safflower oil.

Another embodiment comprises cyclosporin A, Barre Common Degras®, and Safflower oil.

Another embodiment comprises cyclosporin A, Cholesterol NF®, and Safflower oil.

Another embodiment comprises cyclosporin A, Crodalan AWS®, and Safflower oil.

Another embodiment comprises cyclosporin A, Crodalan LA®, and Safflower oil.

Another embodiment comprises cyclosporin A, Emery 1650®, and Safflower oil.

Another embodiment comprises cyclosporin A, Emery 1650®, and Safflower oil.

Another embodiment comprises cyclosporin A, Emery 1740®, and Safflower oil.

Another embodiment comprises cyclosporin A, Emery 1740®, and Safflower oil.

Another embodiment comprises cyclosporin A, Forlan 500®, and Safflower oil.

Another embodiment comprises cyclosporin A, Forlan L®, and Safflower oil.

Another embodiment comprises cyclosporin A, Laneto 50®, and Safflower oil.

Another embodiment comprises cyclosporin A, Lanetd 100®, and Safflower oil.

Another embodiment comprises cyclosporin A, Laneto AWS®, and Safflower oil.

Another embodiment comprises cyclosporin A, Lanfrax 1776®, and Safflower oil.

Another embodiment comprises cyclosporin A, Lanfrax 1776®, and Safflower oil.

Another embodiment comprises cyclosporin A, Lanogel 21®, and Safflower oil.

Another embodiment comprises cyclosporin A, Lipolan®, and Safflower oil.

Another embodiment comprises cyclosporin A, Lipolan 31®, and Safflower oil.

Another embodiment comprises cyclosporin A, OHlan®, and Safflower oil.

Another embodiment comprises cyclosporin A, Polychol 5®, and Safflower oil.

Another embodiment comprises cyclosporin A, Polychol 15®, and Safflower oil.

Another embodiment comprises cyclosporin A, Ritacetyl®, and Safflower oil.

Another embodiment comprises cyclosporin A, Ritachol®, and Safflower oil.

Another embodiment comprises cyclosporin A, Ritahydrox®, and Safflower oil.

Another embodiment comprises cyclosporin A, Ritalafa®, and Safflower oil.

Another embodiment comprises cyclosporin A, Ritalan®, and Safflower oil.

Another embodiment comprises cyclosporin A, Ritalan AWS®, and Safflower oil.

Another embodiment comprises cyclosporin A, Ritalan C®, and Safflower oil.

Another embodiment comprises cyclosporin A, Ritawax®, and Safflower oil.

Another embodiment comprises cyclosporin A, Ritawax AEO®, and Safflower oil.

Another embodiment comprises cyclosporin A, Ritawax ALA®, and Safflower oil.

Another embodiment comprises cyclosporin A, Solan/Solan 50/Super Solan®, and Safflower oil.

Another embodiment comprises cyclosporin A, Super Hartolan/Hartolan®, and Safflower oil.

Another embodiment comprises cyclosporin A, Supersat AWS-4®, and Safflower oil.

Another embodiment comprises cyclosporin A, Supersat AWS-24®, and Safflower oil.

Another embodiment comprises cyclosporin A, a Lecithin or Lecithin Derivative, and Safflower oil.

Another embodiment comprises cyclosporin A, Alcolec®, and Safflower oil.

Another embodiment comprises cyclosporin A, Lecithin®, and Safflower oil.

Another embodiment comprises cyclosporin A, Lexin K®, and Safflower oil.

Another embodiment comprises cyclosporin A, Natipide®, and Safflower oil.

Another embodiment comprises cyclosporin A, a Lignin or Lignin Derivative, and Safflower oil.

Another embodiment comprises cyclosporin A, Diwatex XP 9®, and Safflower oil.

Another embodiment comprises cyclosporin A, Dynasperse LCD®, and Safflower oii.

Another embodiment comprises cyclosporin A, Indulin SAL®, and Safflower oil.

Another embodiment comprises cyclosporin A, Indulin W-1®, and Safflower oil.

Another embodiment comprises cyclosporin A, Indulin W-5®, and Safflower oil.

Another embodiment comprises cyclosporin A, Lignosol FTA®, and Safflower oil.

Another embodiment comprises cyclosporin A, Lignosol SFX-65®, and Safflower oil.

Another embodiment comprises cyclosporin A, Marasperse 52 CP®, and Safflower oil.

Another embodiment comprises cyclosporin A, Marasperse AG®, and Safflower oil.

Another embodiment comprises cyclosporin A, Marasperse CBOS-4®, and Safflower oil.

Another embodiment comprises cyclosporin A, Ufoxane 2®, and Safflower oil.

Another embodiment comprises cyclosporin A, a Methyl Ester, and Safflower oil.

Another embodiment comprises cyclosporin A, E.B. Cleaner AK®, and Safflower oil.

Another embodiment comprises cyclosporin A, Oleocal ME-70®, and Safflower oil.

Another embodiment comprises cyclosporin A, Trade Name Company®, and Safflower oil.

Another embodiment comprises cyclosporin A, Methyl Esters (cont'd)®, and Safflower oil.

Another embodiment comprises cyclosporin A, Oleocal ME-92®, and Safflower oil.

Another embodiment comprises cyclosporin A, Oleocal ME-112®, and Safflower oil.

Another embodiment comprises cyclosporin A, Oleocal ME-130®, and Safflower oil.

Another embodiment comprises cyclosporin A, a Monoglyceride or a Derivative thereof. and Safflower oil.

Another embodiment comprises cyclosporin A, Dynacet 211®, and Safflower oil.

Another embodiment comprises cyclosporin A, Hetsorb S-20®, and Safflower oil.

Another embodiment comprises cyclosporin A, lmwitor 191®, and Safflower oil.

Another embodiment comprises cyclosporin A, Imwitor 370®, and Safflower oil.

Another embodiment comprises cyclosporin A, lmwitor 375®, and Safflower oil.

Another embodiment comprises cyclosporin A, lmwitor 900®, and Safflower oil.

Another embodiment comprises cyclosporin A, lmwitor 945®, and Safflower oil.

Another embodiment comprises cyclosporin A, lmwitor 2020®, and Safflower oil.

Another embodiment comprises cyclosporin A, Kemester 5500®, and Safflower oil.

Another embodiment comprises cyclosporin A, Kemester 6000®, and Safflower oil.

Another embodiment comprises cyclosporin A, Magrabar GMC®, and Safflower oil.

Another embodiment comprises cyclosporin A, Magrabar GMO-CK®, and Safflower oil.

Another embodiment comprises cyclosporin A, Magrabar GPC-10®, and Safflower oil.

Another embodiment comprises cyclosporin A, Magrabar MDG-5050®, and Safflower oil.

Another embodiment comprises cyclosporin A, Monalube 335®, and Safflower oil.

Another embodiment comprises cyclosporin A, Monoglycerides Glyceryl Monestearate Archer Daniels Midland Company®, and Safflower oil.

Another embodiment comprises cyclosporin A, Rita GMS®, and Safflower oil.

Another embodiment comprises cyclosporin A, Ritamulse SCG®, and Safflower oil.

Another embodiment comprises cyclosporin A, Softigen 701®, and Safflower oil.

Another embodiment comprises cyclosporin A, Tally 100 Plus®, and Safflower oil.

Another embodiment comprises cyclosporin A, a Polyethylene Glycol, and Safflower oil.

Another embodiment comprises cyclosporin A, Emulgade PL 68/50®, and Safflower oil.

Another embodiment comprises cyclosporin A, Lumulse PEG®, and Safflower oil.

Another embodiment comprises cyclosporin A, Rhodasurf PEG-400®, and Safflower oil.

Another embodiment comprises cyclosporin A, Rhodasud PEG-600®, and Safflower oil.

Another embodiment comprises cyclosporin A, Witconol PEG-400®, and Safflower oil.

Another embodiment comprises cyclosporin A, a Polymeric Surfactant, and Safflower oil.

Another embodiment comprises cyclosporin A, Acritamer PNC-EG®, and Safflower oil.

Another embodiment comprises cyclosporin A, Ag-Rho DEP-775®, and Safflower oil.

Another embodiment comprises cyclosporin A, APG 325N Glycoside®, and Safflower oil.

Another embodiment comprises cyclosporin A, Aristoflex AVC®, and Safflower oil.

Another embodiment comprises cyclosporin A, Aristoflex HMB®, and Safflower oil.

Another embodiment comprises cyclosporin A, Burco NPS-225®, and Safflower oil.

Another embodiment comprises cyclosporin A, Burco NPS-816®, and Safflower oil.

Another embodiment comprises cyclosporin A, Chemccinate 5603®, and Safflower oil.

Another embodiment comprises cyclosporin A, Cosmedia Guar C-261N®, and Safflower oil.

Another embodiment comprises cyclosporin A, Gantrez S-95®, and Safflower oil.

Another embodiment comprises cyclosporin A, Glucopon 220 UP®, and Safflower oil.

Another embodiment comprises cyclosporin A, Glucopon 225 DK®, and Safflower oil.

Another embodiment comprises cyclosporin A, Glucopon 425 N®, and Safflower oil.

Another embodiment comprises cyclosporin A, Glucopon 600 UP®, and Safflower oil.

Another embodiment comprises cyclosporin A, Glucopon 625 UP®, and Safflower oil.

Another embodiment comprises cyclosporin A, Pemulen 1621®, and Safflower oil.

Another embodiment comprises cyclosporin A, Pemulen 1622®, and Safflower oil.

Another embodiment comprises cyclosporin A, Pemulen TR-1®, and Safflower oil.

Another embodiment comprises cyclosporin A, Pemulen TR-2®, and Safflower oil.

Another embodiment comprises cyclosporin A, Plantacare 818®, and Safflower oil.

Another embodiment comprises cyclosporin A, Plantapon LGC Sorb®, and Safflower oil.

Another embodiment comprises cyclosporin A, Plantaren 1200N®, and Safflower oil.

Another embodiment comprises cyclosporin A, Plantaren 2000N®, and Safflower oil.

Another embodiment comprises cyclosporin A, Viscolam AT 64®, and Safflower oil.

Another embodiment comprises cyclosporin A, Viscolam AT 64P®, and Safflower oil.

Another embodiment comprises cyclosporin A, Viscolam AT 100®, and Safflower oil.

Another embodiment comprises cyclosporin A, Viscolam MAC 7®, and Safflower oil.

Another embodiment comprises cyclosporin A, Viscolam SMC 20®, and Safflower oil.

Another embodiment comprises cyclosporin A, Witbreak RTC-323®, and Safflower oil.

Another embodiment comprises cyclosporin A, WSI 3700®, and Safflower oil.

Another embodiment comprises cyclosporin A, a Propoxylated or Ethoxylated Fatty Acid, Alcohol, or Alkyl Phenol, and 35 Safflower oil.

Another embodiment comprises cyclosporin A, Antarox AA-60®, and Safflower oil.

Another embodiment comprises cyclosporin A, Antarox LF-224®, and Safflower oil.

Another embodiment comprises cyclosporin A, Burcomul DFE-45®, and Safflower oil.

Another embodiment comprises cyclosporin A, Burcoterge LFE-1000®, and Safflower oil.

Another embodiment comprises cyclosporin A, Chemal LF-25B®, and Safflower oil.

Another embodiment comprises cyclosporin A, Chemal LF-40B®, and Safflower oil.

Another embodiment comprises cyclosporin A, Dehypon LS-36®, and Safflower oil.

Another embodiment comprises cyclosporin A, Dehypon LS-36®, and Safflower oil.

Another embodiment comprises cyclosporin A, Dehypon LS-54®, and Safflower oil.

Another embodiment comprises cyclosporin A, Dehypon LS-54®, and Safflower oil.

Another embodiment comprises cyclosporin A, Delonic 100 VLF®, and Safflower oil.

Another embodiment comprises cyclosporin A, Delonic LF-60 MOD®, and Safflower oil.

Another embodiment comprises cyclosporin A, Empiderm B®, and Safflower oil.

Another embodiment comprises cyclosporin A, Ethylan 1206®, and Safflower oil.

Another embodiment comprises cyclosporin A, Ethylan NS-500K®, and Safflower oil.

Another embodiment comprises cyclosporin A, Ethylan NS-500LQ®, and Safflower oil.

Another embodiment comprises cyclosporin A, Genapol 1392®, and Safflower oil.

Another embodiment comprises cyclosporin A, Genapol 2317®, and Safflower oil.

Another embodiment comprises cyclosporin A, Genapol 26EP710®, and Safflower oil.

Another embodiment comprises cyclosporin A, Genapol EP 1022®, and Safflower oil.

Another embodiment comprises cyclosporin A, Genapol EP 1024®, and Safflower oil.

Another embodiment comprises cyclosporin A, Genapol EP 6068®, and Safflower oil.

Another embodiment comprises cyclosporin A, Genapol NP915®, and Safflower oil.

Another embodiment comprises cyclosporin A, Kieralon MFB®, and Safflower oil.

Another embodiment comprises cyclosporin A, Lumisolve CSA-80 V®, and Safflower oil.

Another embodiment comprises cyclosporin A, Marlowet 5001®, and Safflower oil.

Another embodiment comprises cyclosporin A, Marlox FK 64®, and Safflower oil.

Another embodiment comprises cyclosporin A, Marlox MO 124®, and Safflower oil.

Another embodiment comprises cyclosporin A, Marlox S 58®, and Safflower oil.

Another embodiment comprises cyclosporin A, Nonatell 1003®, and Safflower oil.

Another embodiment comprises cyclosporin A, Nonatell 1038®, and Safflower oil.

Another embodiment comprises cyclosporin A, Nonatell 1052®, and Safflower oil.

Another embodiment comprises cyclosporin A, Nonatell 1061®, and Safflower oil.

Another embodiment comprises cyclosporin A, Nonatell 1075®, and Safflower oil.

Another embodiment comprises cyclosporin A, Nonatell 1088®, and Safflower oil.

Another embodiment comprises cyclosporin A, Nonatell 1123®, and Safflower oil.

Another embodiment comprises cyclosporin A, Nonatell 1153®, and Safflower oil.

Another embodiment comprises cyclosporin A, Nonatell 1161®, and Safflower oil.

Another embodiment comprises cyclosporin A, Nonatell 1172®, and Safflower oil.

Another embodiment comprises cyclosporin A, Nonatell 1181®, and Safflower oil.

Another embodiment comprises cyclosporin A, Norfox 36®, and Safflower oil.

Another embodiment comprises cyclosporin A, Procetyl AWS®, and Safflower oil.

Another embodiment comprises cyclosporin A, Sandoxylate SX 412®, and Safflower oil.

Another embodiment comprises cyclosporin A, Sandoxylate SX 418®, and Safflower oil.

Another embodiment comprises cyclosporin A, Surfonic JL-80X®, and Safflower oil.

Another embodiment comprises cyclosporin A, Surfonic JL-80X-B1®, and Safflower oil.

Another embodiment comprises cyclosporin A, Surfonic L4-29X®, and Safflower oil.

Another embodiment comprises cyclosporin A, Surfonic LF®, and Safflower oil.

Another embodiment comprises cyclosporin A, T-Det A826®, and Safflower oil.

Another embodiment comprises cyclosporin A, T-Det LF-416®, and Safflower oil.

Another embodiment comprises cyclosporin A, Tergitol Min-Foam 1X®, and Safflower oil.

Another embodiment comprises cyclosporin A, Tergitol Min-Foam 2X®, and Safflower oil.

Another embodiment comprises cyclosporin A, Triton CF-21®, and Safflower oil.

Another embodiment comprises cyclosporin A, Triton CF-76®, and Safflower oil.

Another embodiment comprises cyclosporin A, Triton XL-80N®, and Safflower oil.

Another embodiment comprises cyclosporin A, Witconol NS-98®, and Safflower oil.

Another embodiment comprises cyclosporin A, Witconol NS-108LQ®, and Safflower oil.

Another embodiment comprises cyclosporin A, Witconol NS-145®, and Safflower oil.

Another embodiment comprises cyclosporin A, Witconol NS-179®, and Safflower oil.

Another embodiment comprises cyclosporin A, a Protein-based Surfactant, and Safflower oil.

Another embodiment comprises cyclosporin A, AminoFoam W®, and Safflower oil.

Another embodiment comprises cyclosporin A, Amiter LGOD-2®, and Safflower oil.

Another embodiment comprises cyclosporin A, Amiter LGS-2®, and Safflower oil.

Another embodiment comprises cyclosporin A, Amiter LGS-5®, and Safflower oil.

Another embodiment comprises cyclosporin A, Lamepon S®, and Safflower oil.

Another embodiment comprises cyclosporin A, Lamepon S®, and Safflower oil.

Another embodiment comprises cyclosporin A, Maypon 4C®, and Safflower oil.

Another embodiment comprises cyclosporin A, May-Tein C®, and Safflower oil.

Another embodiment comprises cyclosporin A, May-Tein CT®, and Safflower oil.

Another embodiment comprises cyclosporin A, May-Tein KTS®, and Safflower oil.

Another embodiment comprises cyclosporin A, May-Tein SY®, and Safflower oil.

Another embodiment comprises cyclosporin A, Plantapon S®, and Safflower oil.

Another embodiment comprises cyclosporin A, Proteol APL®, and Safflower oil.

Another embodiment comprises cyclosporin A, Proteol OAT®, and Safflower oil.

Another embodiment comprises cyclosporin A, Pyroter CPI-40®, and Safflower oil.

Another embodiment comprises cyclosporin A, Pyroter GPI-25®, and Safflower oil.

Another embodiment comprises cyclosporin A, Supro-Tein S®, and Safflower oil.

Another embodiment comprises cyclosporin A, Supro-Tein V®, and Safflower oil.

Another embodiment comprises cyclosporin A,® and Safflower oil.

Another embodiment comprises cyclosporin A, a Sarcosine Derivative®, and Safflower oil.

Another embodiment comprises cyclosporin A, Crodasinic LS-30®, and Safflower oil.

Another embodiment comprises cyclosporin A, Vanseal CS®, and Safflower oil.

Another embodiment comprises cyclosporin A, Vanseal LS®, and Safflower oil.

Another embodiment comprises cyclosporin A, Vanseal MS®, and Safflower oil.

Another embodiment comprises cyclosporin A, Vanseal NACS-30®, and Safflower oil.

Another embodiment comprises cyclosporin A, Vanseal NALS-95®, and Safflower oil.

Another embodiment comprises cyclosporin A, Vanseal OS®, and Safflower oil.

Another embodiment comprises cyclosporin A,® and Safflower oil.

Another embodiment comprises cyclosporin A, a Silicone-based Surfactants, and Safflower oil.

Another embodiment comprises cyclosporin A, Abil-B-9950®, and Safflower oil.

Another embodiment comprises cyclosporin A, Abil Care 85®, and Safflower oil.

Another embodiment comprises cyclosporin A, Abil EM 90®, and Safflower oil.

Another embodiment comprises cyclosporin A, Abil EM 97®, and Safflower oil.

Another embodiment comprises cyclosporin A, Abil WE-09®, and Safflower oil.

Another embodiment comprises cyclosporin A, Dow Corning 1248 Fluid®, and Safflower oil.

Another embodiment comprises cyclosporin A, Dow Corning 3225C®, and Safflower oil.

Another embodiment comprises cyclosporin A, Dow Corning 5200®, and Safflower oil.

Another embodiment comprises cyclosporin A, Dow Corning Q4-3667®, and Safflower oil.

Another embodiment comprises cyclosporin A, Monasil PCA®, and Safflower oil.

Another embodiment comprises cyclosporin A, Monasil PDM®, and Safflower oil.

Another embodiment comprises cyclosporin A, Monasil PLN®, and Safflower oil.

Another embodiment comprises cyclosporin A, Polyderm PPI-SI-WS®, and Safflower oil.

Another embodiment comprises cyclosporin A, Troysol 380W®, and Safflower oil.

Another embodiment comprises cyclosporin A, Troysol S366®, and Safflower oil.

Another embodiment comprises cyclosporin A, a Sorbitan Derivative, and Safflower oil.

Another embodiment comprises cyclosporin A, Alkamuls SML®, and Safflower oil.

Another embodiment comprises cyclosporin A, Alkamuls SMO®, and Safflower oil.

Another embodiment comprises cyclosporin A, Alkamuls STO®, and Safflower oil.

Another embodiment comprises cyclosporin A, Arlacel 20®, and Safflower oil.

Another embodiment comprises cyclosporin A, Arlacel 40®, and Safflower oil.

Another embodiment comprises cyclosporin A, Arlacel 60®, and Safflower oil.

Another embodiment comprises cyclosporin A, Arlacel 80®, and Safflower oil.

Another embodiment comprises cyclosporin A, Arlacel C®, and Safflower oil.

Another embodiment comprises cyclosporin A, Armul 21®, and Safflower oil.

Another embodiment comprises cyclosporin A, Atlox 80®, and Safflower oil.

Another embodiment comprises cyclosporin A, Atlox 847®, and Safflower oil.

Another embodiment comprises cyclosporin A, Atlox 1045A®, and Safflower oil.

Another embodiment comprises cyclosporin A, Canarcel 20®, and Safflower oil.

Another embodiment comprises cyclosporin A, Canarcel 60®, and Safflower oil.

Another embodiment comprises cyclosporin A, Canarcel 80®, and Safflower oil.

Another embodiment comprises cyclosporin A, Canarcel TW 20®, and Safflower oil.

Another embodiment comprises cyclosporin A, Canarcel TW 60®, and Safflower oil.

Another embodiment comprises cyclosporin A, Canarcel TW 80®, and Safflower oil.

Another embodiment comprises cyclosporin A, Coladet BSB-P®, and Safflower oil.

Another embodiment comprises cyclosporin A, Customulse O-20®, and Safflower oil.

Another embodiment comprises cyclosporin A, Dehymuls E®, and Safflower oil.

Another embodiment comprises cyclosporin A, DeSotan SMO®, and Safflower oil.

Another embodiment comprises cyclosporin A, DeSotan SMO-20®, and Safflower oil.

Another embodiment comprises cyclosporin A, DeSotan SMT®, and Safflower oil.

Another embodiment comprises cyclosporin A, DeSotan SMT-20®, and Safflower oil.

Another embodiment comprises cyclosporin A, Durfax 60®, and Safflower oil.

Another embodiment comprises cyclosporin A, Durfax 65®, and Safflower oil.

Another embodiment comprises cyclosporin A, Durfax 80®, and Safflower oil.

Another embodiment comprises cyclosporin A, Durtan 60®, and Safflower oil.

Another embodiment comprises cyclosporin A, Durtan 65®, and Safflower oil.

Another embodiment comprises cyclosporin A, Liposorb L®, and Safflower oil.

Another embodiment comprises cyclosporin A, Liposorb L-10®, and Safflower oil.

Another embodiment comprises cyclosporin A, Liposorb L-20®, and Safflower oil.

Another embodiment comprises cyclosporin A, Liposorb O®, and Safflower oil.

Another embodiment comprises cyclosporin A, Liposorb O-20®, and Safflower oil.

Another embodiment comprises cyclosporin A, Liposorb P®, and Safflower oil.

Another embodiment comprises cyclosporin A, Liposorb P-20®, and Safflower oil.

Another embodiment comprises cyclosporin A, Liposorb S®, and Safflower oil.

Another embodiment comprises cyclosporin A, Liposorb S-20®, and Safflower oil.

Another embodiment comprises cyclosporin A, Liposorb SQO®, and Safflower oil.

Another embodiment comprises cyclosporin A, Liposorb TO®, and Safflower oil.

Another embodiment comprises cyclosporin A, Liposorb TS®, and Safflower oil.

Another embodiment comprises cyclosporin A, Liposorb TS-20®, and Safflower oil.

Another embodiment comprises cyclosporin A, Lumisorb PS®, and Safflower oil.

Another embodiment comprises cyclosporin A, Lumisorb SMO (T)®, and Safflower oil.

Another embodiment comprises cyclosporin A, Lumisorb SMS K®, and Safflower oil.

Another embodiment comprises cyclosporin A, Lumisorb SSO®, and Safflower oil.

Another embodiment comprises cyclosporin A, Lumisorb STS K®, and Safflower oil.

Another embodiment comprises cyclosporin A, Lumisorb STT®, and Safflower oil.

Another embodiment comprises cyclosporin A, Magrabar SMO®, and Safflower oil.

Another embodiment comprises cyclosporin A, Magrabar SMO-VEG®, and Safflower oil.

Another embodiment comprises cyclosporin A, Magrabar SMT®, and Safflower oil.

Another embodiment comprises cyclosporin A, Magrabar STO®, and Safflower oil.

Another embodiment comprises cyclosporin A, Miracare BC-27®, and Safflower oil.

Another embodiment comprises cyclosporin A, Ritabate 20®, and Safflower oil.

Another embodiment comprises cyclosporin A, Ritabate 40®, and Safflower oil.

Another embodiment comprises cyclosporin A, Ritabate 60®, and Safflower oil.

Another embodiment comprises cyclosporin A, Ritabate 80®, and Safflower oil.

Another embodiment comprises cyclosporin A, T-Maz®, and Safflower oil.

Another embodiment comprises cyclosporin A, Tego SML®, and Safflower oil.

Another embodiment comprises cyclosporin A, Tego SML 20®, and Safflower oil.

Another embodiment comprises cyclosporin A, Tego SMO 80 V®, and Safflower oil.

Another embodiment comprises cyclosporin A, Tego SMO V®, and Safflower oil.

Another embodiment comprises cyclosporin A, Tego SMS®, and Safflower oil.

Another embodiment comprises cyclosporin A, Tego STO V®, and Safflower oil.

Another embodiment comprises cyclosporin A, Tween 21®, and Safflower oil.

Another embodiment comprises cyclosporin A, Tween 40®, and Safflower oil.

Another embodiment comprises cyclosporin A, Tween 60®, and Safflower oil.

Another embodiment comprises cyclosporin A, Tween 60 K®, and Safflower oil.

Another embodiment comprises cyclosporin A, Tween 61®, and Safflower oil.

Another embodiment comprises cyclosporin A, Tween 65®, and Safflower oil.

Another embodiment comprises cyclosporin A, Tween 80®, and Safflower oil.

Another embodiment comprises cyclosporin A, Tween 80 K®, and Safflower oil.

Another embodiment comprises cyclosporin A, Tween 81®, and Safflower oil.

Another embodiment comprises cyclosporin A, Tween 85®, and Safflower oil.

Another embodiment comprises cyclosporin A, a Sucrose or Glucose Ester, or Derivative thereof, and Safflower oil.

Another embodiment comprises cyclosporin A, DeSulf GOS-P-60WCG®, and Safflower oil.

Another embodiment comprises cyclosporin A, Glucam E-20 Distearate®, and Safflower oil.

Another embodiment comprises cyclosporin A, Glucamate DOE-120®, and Safflower oil.

Another embodiment comprises cyclosporin A, Glucamate SSE-20®, and Safflower oil.

Another embodiment comprises cyclosporin A, Glucate DO®, and Safflower oil.

Another embodiment comprises cyclosporin A, Glucate SS®, and Safflower oil.

Another embodiment comprises cyclosporin A, Glucopon 425 UP®, and Safflower oil.

Another embodiment comprises cyclosporin A, Isolan IS®, and Safflower oil.

Another embodiment comprises cyclosporin A, Mazon 40®, and Safflower oil.

Another embodiment comprises cyclosporin A, Montanov 82®, and Safflower oil.

Another embodiment comprises cyclosporin A, Montanov 202®, and Safflower oil.

Another embodiment comprises cyclosporin A, Montanov S®, and Safflower oil.

Another embodiment comprises cyclosporin A, Rheozan®, and Safflower oil.

Another embodiment comprises cyclosporin A, Simulsol AS 48®, and Safflower oil.

Another embodiment comprises cyclosporin A, Simulsol SL 4®, and Safflower oil.

Another embodiment comprises cyclosporin A, Simulsol SL 10®, and Safflower oil.

Another embodiment comprises cyclosporin A, Simulsol SL 11 V®, and Safflower oil.

Another embodiment comprises cyclosporin A, Simulsol SL 55®, and Safflower oil.

Another embodiment comprises cyclosporin A, Suga Nate 100 and 160®, and Safflower oil.

Another embodiment comprises cyclosporin A, Tego Care 450®, and Safflower oil.

Another embodiment comprises cyclosporin A, Tego Care CG 90®, and Safflower oil.

Another embodiment comprises cyclosporin A, Tego Care PS®, and Safflower oil.

Another embodiment comprises cyclosporin A, Tegosoft PSE 141 G®, and Safflower oil.

Another embodiment comprises cyclosporin A, Tegotens G 826®, and Safflower oil.

Another embodiment comprises cyclosporin A, Triton BG-10 (70%)®, and Safflower oil.

Another embodiment comprises cyclosporin A, Triton CG-110 (60%)®, and Safflower oil.

Another embodiment comprises cyclosporin A, Wickenol 545®, and Safflower oil.

Another embodiment comprises cyclosporin A, an alcohol, and Maize oil.

Another embodiment comprises cyclosporin A, Diglycerol®, and Maize oil.

Another embodiment comprises cyclosporin A, Hetoxide GT-80®, and Maize oil.

Another embodiment comprises cyclosporin A, Lexemul BEO®, and Maize oil.

Another embodiment comprises cyclosporin A, Polyglycerol-3®, and Maize oil.

Another embodiment comprises cyclosporin A, Redicote E Series®, and Maize oil.

Another embodiment comprises cyclosporin A, Simulsol OX 1005L®, and Maize oil.

Another embodiment comprises cyclosporin A, Stanfax 567®, and Maize oil.

Another embodiment comprises cyclosporin A, TA-1618®, and Maize oil.

Another embodiment comprises cyclosporin A, Witconol H-31A®, and Maize oil.

Another embodiment comprises cyclosporin A, an amine oxide, and Maize oil.

Another embodiment comprises cyclosporin A, AO-405®, and Maize oil.

Another embodiment comprises cyclosporin A, AO-455®, and Maize oil.

Another embodiment comprises cyclosporin A, AO 728 Special®, and Maize oil.

Another embodiment comprises cyclosporin A, Barlox 12®, and Maize oil.

Another embodiment comprises cyclosporin A, Barlox 14®, and Maize oil.

Another embodiment comprises cyclosporin A, Burcoxide Lo®, and Maize oil.

Another embodiment comprises cyclosporin A, Caloxamine LO®, and Maize oil.

Another embodiment comprises cyclosporin A, Chemoxide CAW®, and Maize oil.

Another embodiment comprises cyclosporin A, Chemoxide LM-30®, and Maize oil.

Another embodiment comprises cyclosporin A, Chemoxide LO®, and Maize oil.

Another embodiment comprises cyclosporin A, Chemoxide MO®, and Maize oil.

Another embodiment comprises cyclosporin A, Colalux CAO-35®, and Maize oil.

Another embodiment comprises cyclosporin A, Colalux LO®, and Maize oil.

Another embodiment comprises cyclosporin A, DeMox CAPO®, and Maize oil.

Another embodiment comprises cyclosporin A, DeMox CSG-30®, and Maize oil.

Another embodiment comprises cyclosporin A, DeMox LAO®, and Maize oil.

Another embodiment comprises cyclosporin A, Emcol L®, and Maize oil.

Another embodiment comprises cyclosporin A, Empigen OB®, and Maize oil.

Another embodiment comprises cyclosporin A, Empigen OS/A®, and Maize oil.

Another embodiment comprises cyclosporin A, Foamox CDO®, and Maize oil.

Another embodiment comprises cyclosporin A, Foamox DMM®, and Maize oil.

Another embodiment comprises cyclosporin A, Foamox DMS®, and Maize oil.

Another embodiment comprises cyclosporin A, Genaminox KC®, and Maize oil.

Another embodiment comprises cyclosporin A, Genaminox LA®, and Maize oil.

Another embodiment comprises cyclosporin A, Hartofoam SAO®, and Maize oil.

Another embodiment comprises cyclosporin A, Hartox DMCD®, and Maize oil.

Another embodiment comprises cyclosporin A, Lipowax DAT®, and Maize oil.

Another embodiment comprises cyclosporin A, Lipowax PB Pastilles®, and Maize oil.

Another embodiment comprises cyclosporin A, Mackamine C8®, and Maize oil.

Another embodiment comprises cyclosporin A, Mackamine C10®, and Maize oil.

Another embodiment comprises cyclosporin A, Mackamine C14®, and Maize oil.

Another embodiment comprises cyclosporin A, Mackamine CAO®, and Maize oil.

Another embodiment comprises cyclosporin A, Mackamine CO®, and Maize oil.

Another embodiment comprises cyclosporin A, Mackamine LO®, and Maize oil.

Another embodiment comprises cyclosporin A, Mackamine O2®, and Maize oil.

Another embodiment comprises cyclosporin A, Mackamine SAO®, and Maize oil.

Another embodiment comprises cyclosporin A, Mackamine SO®, and Maize oil.

Another embodiment comprises cyclosporin A, Mazox KCAO®, and Maize oil.

Another embodiment comprises cyclosporin A, Monalac MO®, and Maize oil.

Another embodiment comprises cyclosporin A, Norfox LDA®, and Maize oil.

Another embodiment comprises cyclosporin A, Rhodamox LO®, and Maize oil.

Another embodiment comprises cyclosporin A, Schercamox C-AA®, and Maize oil.

Another embodiment comprises cyclosporin A, Schercamox DMA®, and Maize oil.

Another embodiment comprises cyclosporin A, Schercamox DML®, and Maize oil.

Another embodiment comprises cyclosporin A, Schercamox DMM®, and Maize oil.

Another embodiment comprises cyclosporin A, Schercamox DMS®, and Maize oil.

Another embodiment comprises cyclosporin A, Tegotens DO®, and Maize oil.

Another embodiment comprises cyclosporin A, Tomah AO-14-2®, and Maize oil.

Another embodiment comprises cyclosporin A, Triaminox CDO®, and Maize oil.

Another embodiment comprises cyclosporin A, a block polymer and Maize oil.

Another embodiment comprises cyclosporin A, AL 2070®, and Maize oil.

Another embodiment comprises cyclosporin A, Antarox 17-R-2®, and Maize oil.

Another embodiment comprises cyclosporin A, Antarox 25-R-2®, and Maize oil.

Another embodiment comprises cyclosporin A, Antarox 31-R-1®, and Maize oil.

Another embodiment comprises cyclosporin A, Antarox P-84®, and Maize oil.

Another embodiment comprises cyclosporin A, Antarox P-104/H®, and Maize oil.

Another embodiment comprises cyclosporin A, Arnox BP-Series®, and Maize oil.

Another embodiment comprises cyclosporin A, Chemonic 435®, and Maize oil.

Another embodiment comprises cyclosporin A, Chemonic D-25®, and Maize oil.

Another embodiment comprises cyclosporin A, Chemonic PL Series®, and Maize oil.

Another embodiment comprises cyclosporin A, Ethox L-121®, and Maize oil.

Another embodiment comprises cyclosporin A, Ethox L-122®, and Maize oil.

Another embodiment comprises cyclosporin A, Genapol PF-10®, and Maize oil.

Another embodiment comprises cyclosporin A, Genapol PF-20®, and Maize oil.

Another embodiment comprises cyclosporin A, Genapol PF-40A®, and Maize oil.

Another embodiment comprises cyclosporin A, Norfox 2-LF®, and Maize oil.

Another embodiment comprises cyclosporin A, Simulsol NW 342®, and Maize oil.

Another embodiment comprises cyclosporin A, T-Det BP-1®, and Maize oil.

Another embodiment comprises cyclosporin A, T-Det XD®, and Maize oil.

Another embodiment comprises cyclosporin A, T-Det XH®, and Maize oil.

Another embodiment comprises cyclosporin A, Triton CF-32®, and Maize oil.

Another embodiment comprises cyclosporin A, Witconol 171®, and Maize oil.

Another embodiment comprises cyclosporin A, Witconol 324®, and Maize oil.

Another embodiment comprises cyclosporin A, Witconol 324D®, and Maize oil.

Another embodiment comprises cyclosporin A, Witconol PD-2000®, and Maize oil.

Another embodiment comprises cyclosporin A, a Carboxylated Alcohol or Alkylphenol Ethoxylate, and Maize oil.

Another embodiment comprises cyclosporin A, Emcol CN-6®, and Maize oil.

Another embodiment comprises cyclosporin A, Ethcarb®, and Maize oil.

Another embodiment comprises cyclosporin A, Gemtex WNT-Conc®, and Maize oil.

Another embodiment comprises cyclosporin A, Incrodet TD7-C®, and Maize oil.

Another embodiment comprises cyclosporin A, Marlinat CM 105/80®, and Maize oil.

Another embodiment comprises cyclosporin A, Marlowet 1072®, and Maize oil.

Another embodiment comprises cyclosporin A, Marlowet 4530®, and Maize oil.

Another embodiment comprises cyclosporin A, Marlowet 4530 LF®, and Maize oil.

Another embodiment comprises cyclosporin A, Marlowet 4534®, and Maize oil.

Another embodiment comprises cyclosporin A, Marlowet 4538®, and Maize oil.

Another embodiment comprises cyclosporin A, Marlowet 4539®, and Maize oil.

Another embodiment comprises cyclosporin A, Marlowet 4539 LF®, and Maize oil.

Another embodiment comprises cyclosporin A, Marlowet 4541®, and Maize oil.

Another embodiment comprises cyclosporin A, Miranate LEC-80®, and Maize oil.

Another embodiment comprises cyclosporin A, Sandopan B®, and Maize oil.

Another embodiment comprises cyclosporin A, Sandopan B Modified®, and Maize oil.

Another embodiment comprises cyclosporin A, Sandopan LS-24 Gel®, and Maize oil.

Another embodiment comprises cyclosporin A, Surtine T-A®, and Maize oil.

Another embodiment comprises cyclosporin A, a Carboxylic Acid/Fatty Acid. and Maize oil.

Another embodiment comprises cyclosporin A, Colaterge RAM®, and Maize oil.

Another embodiment comprises cyclosporin A, Colatrope INC®, and Maize oil.

Another embodiment comprises cyclosporin A, Crodacid B®, and Maize oil.

Another embodiment comprises cyclosporin A, DeTrope CA-100®, and Maize oil.

Another embodiment comprises cyclosporin A, Latol MTO®, and Maize oil.

Another embodiment comprises cyclosporin A, Lumulse CC-33 K®, and Maize oil.

Another embodiment comprises cyclosporin A, Mulls 2218®, and Maize oil.

Another embodiment comprises cyclosporin A, OL-600®, and Maize oil.

Another embodiment comprises cyclosporin A, OL-800®, and Maize oil.

Another embodiment comprises cyclosporin A, R-910®, and Maize oil.

Another embodiment comprises cyclosporin A, S-210®, and Maize oil.

Another embodiment comprises cyclosporin A, Sandopan DTC Acid®, and Maize oil.

Another embodiment comprises cyclosporin A, Sandopan LS 24 N®, and Maize oil.

Another embodiment comprises cyclosporin A, Sandopan MA-18®, and Maize oil.

Another embodiment comprises cyclosporin A, an Ethoxylated Alcohol, and Maize oil.

Another embodiment comprises cyclosporin A, Adsee 799®, and Maize oil.

Another embodiment comprises cyclosporin A, Adsee 799®, and Maize oil.

Another embodiment comprises cyclosporin A, Alfonic 610-3.5®, and Maize oil.

Another embodiment comprises cyclosporin A, Alfonic 810-2®, and Maize oil.

Another embodiment comprises cyclosporin A, Alfonic 810-6®, and Maize oil.

Another embodiment comprises cyclosporin A, Alfonic 1012-3®, and Maize oil.

Another embodiment comprises cyclosporin A, Alfonic 1012-5®, and Maize oil.

Another embodiment comprises cyclosporin A, Alfonic 1216CO-1.5®, and Maize oil.

Another embodiment comprises cyclosporin A, Alfonic 1216CO-7®, and Maize oil.

Another embodiment comprises cyclosporin A, Alfonic 1412-3®, and Maize oil.

Another embodiment comprises cyclosporin A, Alfonic 1412-7®, and Maize oil.

Another embodiment comprises cyclosporin A, Arlasolve 200®, and Maize oil.

Another embodiment comprises cyclosporin A, Arlasolve 200 Liquid®, and Maize oil.

Another embodiment comprises cyclosporin A, Armix 180-C®, and Maize oil.

Another embodiment comprises cyclosporin A, Armix 183®, and Maize oil.

Another embodiment comprises cyclosporin A, Armul 2404®, and Maize oil.

Another embodiment comprises cyclosporin A, Armul 2404®, and Maize oil.

Another embodiment comprises cyclosporin A, Atlas EMJ-C®, and Maize oil.

Another embodiment comprises cyclosporin A, Atlas G-2109®, and Maize oil.

Another embodiment comprises cyclosporin A, Atlas G-3886®, and Maize oil.

Another embodiment comprises cyclosporin A, Atlas G-3890®, and Maize oil.

Another embodiment comprises cyclosporin A, Bio Soft E-200®, and Maize oil.

Another embodiment comprises cyclosporin A, Bio Soft E-300®, and Maize oil.

Another embodiment comprises cyclosporin A, Bio Soft E-400®, and Maize oil.

Another embodiment comprises cyclosporin A, Bio Soft EN 600®, and Maize oil.

Another embodiment comprises cyclosporin A, Bio Soft TD-400®, and Maize oil.

Another embodiment comprises cyclosporin A, Bio Soft TD-630®, and Maize oil.

Another embodiment comprises cyclosporin A, Brij 30®, and Maize oil.

Another embodiment comprises cyclosporin A, Brij 52®, and Maize oil.

Another embodiment comprises cyclosporin A, Brij 56®, and Maize oil.

Another embodiment comprises cyclosporin A, Brij 58®, and Maize oil.

Another embodiment comprises cyclosporin A, Brij 72®, and Maize oil.

Another embodiment comprises cyclosporin A, Brij 76®, and Maize oil.

Another embodiment comprises cyclosporin A, Brij 78®, and Maize oil.

Another embodiment comprises cyclosporin A, Brij 93®, and Maize oil.

Another embodiment comprises cyclosporin A, Brij 97®, and Maize oil.

Another embodiment comprises cyclosporin A, Brij 98®, and Maize oil.

Another embodiment comprises cyclosporin A, Brij 700®, and Maize oil.

Another embodiment comprises cyclosporin A, Brij 700 S®, and Maize oil.

Another embodiment comprises cyclosporin A, Brij 721®, and Maize oil.

Another embodiment comprises cyclosporin A, Brij 721 S®, and Maize oil.

Another embodiment comprises cyclosporin A, Burcoterge CDG®, and Maize oil.

Another embodiment comprises cyclosporin A, Canasol AT 600®, and Maize oil.

Another embodiment comprises cyclosporin A, Canasol AT 800®, and Maize oil.

Another embodiment comprises cyclosporin A, Canasol AT 1200®, and Maize oil.

Another embodiment comprises cyclosporin A, Canasol BJ 35®, and Maize oil.

Another embodiment comprises cyclosporin A, Canasol BJ 36®, and Maize oil.

Another embodiment comprises cyclosporin A, Canasol BJ 52®, and Maize oil.

Another embodiment comprises cyclosporin A, Canasol BJ 58®, and Maize oil.

Another embodiment comprises cyclosporin A, Canasol BJ 72®, and Maize oil.

Another embodiment comprises cyclosporin A, Canasol BJ 78®, and Maize oil.

Another embodiment comprises cyclosporin A, Canasol BJ 98®, and Maize oil.

Another embodiment comprises cyclosporin A, Canasol BJ 307®, and Maize oil.

Another embodiment comprises cyclosporin A, Cerfak 1400®, and Maize oil.

Another embodiment comprises cyclosporin A, Cetomacrogol 1000 BP®, and Maize oil.

Another embodiment comprises cyclosporin A, Chemonic C-2®, and Maize oil.

Another embodiment comprises cyclosporin A, Chemonic C-10®, and Maize oil.

Another embodiment comprises cyclosporin A, Chemonic C-20®, and Maize oil.

Another embodiment comprises cyclosporin A, Chemonic CT-12®, and Maize oil.

Another embodiment comprises cyclosporin A, Chemonic CT-20®, and Maize oil.

Another embodiment comprises cyclosporin A, Chemonic CT-30®, and Maize oil.

Another embodiment comprises cyclosporin A, Chemonic CT-55®, and Maize oil.

Another embodiment comprises cyclosporin A, Chemonic G-7®, and Maize oil.

Another embodiment comprises cyclosporin A, Chemonic G-26®, and Maize oil.

Another embodiment comprises cyclosporin A, Chemonic L-4®, and Maize oil.

Another embodiment comprises cyclosporin A, Chemonic L-7®, and Maize oil.

Another embodiment comprises cyclosporin A, Chemonic L-12®, and Maize oil.

Another embodiment comprises cyclosporin A, Chemonic L-23®, and Maize oil.

Another embodiment comprises cyclosporin A, Chemonic O-2®, and Maize oil.

Another embodiment comprises cyclosporin A, Chemonic O-5®, and Maize oil.

Another embodiment comprises cyclosporin A, Chemonic O-10®, and Maize oil.

Another embodiment comprises cyclosporin A, Chemonic O-20®, and Maize oil.

Another embodiment comprises cyclosporin A, Chemonic S-2®, and Maize oil.

Another embodiment comprises cyclosporin A, Chemonic S-10®, and Maize oil.

Another embodiment comprises cyclosporin A, Chemonic S-20®, and Maize oil.

Another embodiment comprises cyclosporin A, Colamulse FE®, and Maize oil.

Another embodiment comprises cyclosporin A, Cremophor A 20®, and Maize oil.

Another embodiment comprises cyclosporin A, Cremophor SA 2®, and Maize oil.

Another embodiment comprises cyclosporin A, Dehydol 100®, and Maize oil.

Another embodiment comprises cyclosporin A, Dehydol O-4®, and Maize oil.

Another embodiment comprises cyclosporin A, Delonic C-18®, and Maize oil.

Another embodiment comprises cyclosporin A, DeSonic 6T®, and Maize oil.

Another embodiment comprises cyclosporin A, DeSonic 9D®, and Maize oil.

Another embodiment comprises cyclosporin A, DeSonic 9T®, and Maize oil.

Another embodiment comprises cyclosporin A, DeSonic 12D®, and Maize oil.

Another embodiment comprises cyclosporin A, DeSonic 12T®, and Maize oil.

Another embodiment comprises cyclosporin A, DeSonic 15T®, and Maize oil.

Another embodiment comprises cyclosporin A, DeSonic TDA-9®, and Maize oil.

Another embodiment comprises cyclosporin A, DeThox GLG-7®, and Maize oil.

Another embodiment comprises cyclosporin A, DeThox GLG-26®, and Maize oil.

Another embodiment comprises cyclosporin A, DeThox LA-4®, and Maize oil.

Another embodiment comprises cyclosporin A, DeThox LA-23®, and Maize oil.

Another embodiment comprises cyclosporin A, DeThox SA-80®, and Maize oil.

Another embodiment comprises cyclosporin A, Disponil O5®, and Maize oil.

Another embodiment comprises cyclosporin A, Eccoterge EO-41B®, and Maize oil.

Another embodiment comprises cyclosporin A, Empilan KA2.5/90®, and Maize oil.

Another embodiment comprises cyclosporin A, Empilan KA5/90®, and Maize oil.

Another embodiment comprises cyclosporin A, Empilan KM-20®, and Maize oil.

Another embodiment comprises cyclosporin A, Empilan KM-50®, and Maize oil.

Another embodiment comprises cyclosporin A, Empilan L-23®, and Maize oil.

Another embodiment comprises cyclosporin A, Ethylan 25-3®, and Maize oil.

Another embodiment comprises cyclosporin A, Ethylan 1204®, and Maize oil.

Another embodiment comprises cyclosporin A, Ethylan DA-4®, and Maize oil.

Another embodiment comprises cyclosporin A, Ethylan LA-230®, and Maize oil.

Another embodiment comprises cyclosporin A, Ethylan SN®, and Maize oil.

Another embodiment comprises cyclosporin A, Ethylan TD-60®, and Maize oil.

Another embodiment comprises cyclosporin A, Ethylan TD-100®, and Maize oil.

Another embodiment comprises cyclosporin A, Ethylan TD-1407®, and Maize oil.

Another embodiment comprises cyclosporin A, Eumulgin B1®, and Maize oil.

Another embodiment comprises cyclosporin A, Eumulgin B2®, and Maize oil.

Another embodiment comprises cyclosporin A, Eumulgin B3®, and Maize oil.

Another embodiment comprises cyclosporin A, Eumulgin O-10®, and Maize oil.

Another embodiment comprises cyclosporin A, Flo Mo 80/20®, and Maize oil.

Another embodiment comprises cyclosporin A, Flo Mo Low Foam®, and Maize oil.

Another embodiment comprises cyclosporin A, Forlan C-24®, and Maize oil.

Another embodiment comprises cyclosporin A, Genapol 1454®, and Maize oil.

Another embodiment comprises cyclosporin A, Genapol BA-020®, and Maize oil.

Another embodiment comprises cyclosporin A, Genapol BA-040®, and Maize oil.

Another embodiment comprises cyclosporin A, Genapol C-100®, and Maize oil.

Another embodiment comprises cyclosporin A, Genapol DA 060®, and Maize oil.

Another embodiment comprises cyclosporin A, Genapol HS 020®, and Maize oil.

Another embodiment comprises cyclosporin A, Genapol HS 200®, and Maize oil.

Another embodiment comprises cyclosporin A, Genapol ID-040®, and Maize oil.

Another embodiment comprises cyclosporin A, Genapol ID-060®, and Maize oil.

Another embodiment comprises cyclosporin A, Genapol ID-090®, and Maize oil.

Another embodiment comprises cyclosporin A, Genapol LA 010®, and Maize oil.

Another embodiment comprises cyclosporin A, Genapol LA 020®, and Maize oil.

Another embodiment comprises cyclosporin A, Genapol LA 030®, and Maize oil.

Another embodiment comprises cyclosporin A, Genapol LA 040®, and Maize oil.

Another embodiment comprises cyclosporin A, Genapol LA 050®, and Maize oil.

Another embodiment comprises cyclosporin A, Genapol LA 060®, and Maize oil.

Another embodiment comprises cyclosporin A, Genapol LA 070®, and Maize oil.

Another embodiment comprises cyclosporin A, Genapol LA 070S®, and Maize oil.

Another embodiment comprises cyclosporin A, Genapol LA 230®, and Maize oil.

Another embodiment comprises cyclosporin A, Genapol O 020®, and Maize oil.

Another embodiment comprises cyclosporin A, Genapol O 050®, and Maize oil.

Another embodiment comprises cyclosporin A, Genapol O 100®, and Maize oil.

Another embodiment comprises cyclosporin A, Genapol O 200®, and Maize oil.

Another embodiment comprises cyclosporin A, Genapol SA 030®, and Maize oil.

Another embodiment comprises cyclosporin A, Genapol SA 120®, and Maize oil.

Another embodiment comprises cyclosporin A, Genapol T-020®, and Maize oil.

Another embodiment comprises cyclosporin A, Genapol UD-030®, and Maize oil.

Another embodiment comprises cyclosporin A, Genapol UD-050®, and Maize oil.

Another embodiment comprises cyclosporin A, Genapol UD-070®, and Maize oil.

Another embodiment comprises cyclosporin A, Genapol UD-079®, and Maize oil.

Another embodiment comprises cyclosporin A, Genapol UD-080®, and Maize oil.

Another embodiment comprises cyclosporin A, Genapol UD-110®, and Maize oil.

Another embodiment comprises cyclosporin A, Genapol X 030®, and Maize oil.

Another embodiment comprises cyclosporin A, Genapol X 050®, and Maize oil.

Another embodiment comprises cyclosporin A, Genapbl X 060®, and Maize oil.

Another embodiment comprises cyclosporin A, Genapol X 070®, and Maize oil.

Another embodiment comprises cyclosporin A, Genapol X 080®, and Maize oil.

Another embodiment comprises cyclosporin A, Genapol X 100®, and Maize oil.

Another embodiment comprises cyclosporin A, Genapol X159®, and Maize oil.

Another embodiment comprises cyclosporin A, Generol 122 E5®, and Maize oil.

Another embodiment comprises cyclosporin A, Generol 122 PE25®, and Maize oil.

Another embodiment comprises cyclosporin A, Hostacerin T-3®, and Maize oil.

Another embodiment comprises cyclosporin A, Iconol LF 110®, and Maize oil.

Another embodiment comprises cyclosporin A, Incropol CS-20®, and Maize oil.

Another embodiment comprises cyclosporin A, Lexemul CS-20®, and Maize oil.

Another embodiment comprises cyclosporin A, Liponic EG-1®, and Maize oil.

Another embodiment comprises cyclosporin A, Lipowax D®, and Maize oil.

Another embodiment comprises cyclosporin A, Lipowax G®, and Maize oil.

Another embodiment comprises cyclosporin A, Lipowax NI®, and Maize oil.

Another embodiment comprises cyclosporin A, Lipowax P®, and Maize oil.

Another embodiment comprises cyclosporin A, Lipowax P-31®, and Maize oil.

Another embodiment comprises cyclosporin A, Lipowax PR®, and Maize oil.

Another embodiment comprises cyclosporin A, Lumulse CS-20®, and Maize oil.

Another embodiment comprises cyclosporin A, Macol CSA-20®, and Maize oil.

Another embodiment comprises cyclosporin A, Marlox B 24/50®, and Maize oil.

Another embodiment comprises cyclosporin A, Mazawet 77®, and Maize oil.

Another embodiment comprises cyclosporin A, Norfox 1713®, and Maize oil.

Another embodiment comprises cyclosporin A, Norfox 2579®, and Maize oil.

Another embodiment comprises cyclosporin A, Norfox Lo Foam®, and Maize oil.

Another embodiment comprises cyclosporin A, Promulgen D®, and Maize oil.

Another embodiment comprises cyclosporin A, Promulgen G®, and Maize oil.

Another embodiment comprises cyclosporin A, Renex 30®, and Maize oil.

Another embodiment comprises cyclosporin A, Renex 36®, and Maize oil.

Another embodiment comprises cyclosporin A, Rhodasurf A 24®, and Maize oil.

Another embodiment comprises cyclosporin A, Rhodasurf AAE-10®, and Maize oil.

Another embodiment comprises cyclosporin A, Rhodasurf BEH-25®, and Maize oil.

Another embodiment comprises cyclosporin A, Rhodasurf BEH-40®, and Maize oil.

Another embodiment comprises cyclosporin A, Rhodasurf DA 530®, and Maize oil.

Another embodiment comprises cyclosporin A, Rhodasurf DA 630®, and Maize oil.

Another embodiment comprises cyclosporin A, Rhodasun DA 639®, and Maize oil.

Another embodiment comprises cyclosporin A, Rhodasurf LAN-23®, and Maize oil.

Another embodiment comprises cyclosporin A, Rhodasurd ON-870®, and Maize oil.

Another embodiment comprises cyclosporin A, Rhodasurf ON-877®, and Maize oil.

Another embodiment comprises cyclosporin A, Rhodasurf TB-970 FLK®, and Maize oil.

Another embodiment comprises cyclosporin A, Ritacet-20®, and Maize oil.

Another embodiment comprises cyclosporin A, Ritachol 1000®, and Maize oil.

Another embodiment comprises cyclosporin A, Ritachol 2000®, and Maize oil.

Another embodiment comprises cyclosporin A, Ritachol 5000®, and Maize oil.

Another embodiment comprises cyclosporin A, Ritox 35®, and Maize oil.

Another embodiment comprises cyclosporin A, Surfonic DA-4®, and Maize oil.

Another embodiment comprises cyclosporin A, Surfonic DA-6®, and Maize oil.

Another embodiment comprises cyclosporin A, Surfonic L46-7®, and Maize oil.

Another embodiment comprises cyclosporin A, Surfonic POA®, and Maize oil.

Another embodiment comprises cyclosporin A, Synthrapol KB®, and Maize oil.

Another embodiment comprises cyclosporin A, Teginacid®, and Maize oil.

Another embodiment comprises cyclosporin A, Teginacid C®, and Maize oil.

Another embodiment comprises cyclosporin A, Tegotens EC 11®, and Maize oil.

Another embodiment comprises cyclosporin A, Tinegal NA®, and Maize oil.

Another embodiment comprises cyclosporin A, Tomadol 400®, and Maize oil.

Another embodiment comprises cyclosporin A, Tomadol 600®, and Maize oil.

Another embodiment comprises cyclosporin A, Tomadol 900®, and Maize oil.

Another embodiment comprises cyclosporin A, Uniperol O®, and Maize oil.

Another embodiment comprises cyclosporin A, Witconol SN Series®, and Maize oil.

Another embodiment comprises cyclosporin A, an Ethoxylated Alkylphenol, and Maize oil.

Another embodiment comprises.cyclosporin A, Antarox LF-222®, and Maize oil.

Another embodiment comprises cyclosporin A, Atlox 775®, and Maize oil.

Another embodiment comprises cyclosporin A, Caloxylate N-9®, and Maize oil.

Another embodiment comprises cyclosporin A, Canasol NF-1000®, and Maize oil.

Another embodiment comprises cyclosporin A, Canasol NF-3000®, and Maize oil.

Another embodiment comprises cyclosporin A, Canasol NF-3070®, and Maize oil.

Another embodiment comprises cyclosporin A, Canasol OF 1670®, and Maize oil.

Another embodiment comprises cyclosporin A, Canasol OF 2570®, and Maize oil.

Another embodiment comprises cyclosporin A, Canasol OF 4070®, and Maize oil.

Another embodiment comprises cyclosporin A, Chemax DNP-8®, and Maize oil.

Another embodiment comprises cyclosporin A, Chemax DNP-18®, and Maize oil.

Another embodiment comprises cyclosporin A, Chemax DNP-150/50®, and Maize oil.

Another embodiment comprises cyclosporin A, DeSonic 1.5N®, and Maize oil.

Another embodiment comprises cyclosporin A, DeSonic 4N®, and Maize oil.

Another embodiment comprises cyclosporin A, DeSonic 5N®, and Maize oil.

Another embodiment comprises cyclosporin A, DeSonic 6D®, and Maize oil.

Another embodiment comprises cyclosporin A, DeSonic 6N®, and Maize oil.

Another embodiment comprises cyclosporin A, DeSonic 7N®, and Maize oil.

Another embodiment comprises cyclosporin A, DeSonic 9N®, and Maize oil.

Another embodiment comprises cyclosporin A, DeSonic 10D®, and Maize oil.

Another embodiment comprises cyclosporin A, DeSonic 11N®, and Maize oil.

Another embodiment comprises cyclosporin A, DeSonic 12N®, and Maize oil.

Another embodiment comprises cyclosporin A, DeSonic 15N®, and Maize oil.

Another embodiment comprises cyclosporin A, DeSonic 20N®, and Maize oil.

Another embodiment comprises cyclosporin A, Eccoscour RC®, and Maize oil.

Another embodiment comprises cyclosporin A, Eccoterge EO-100®, and Maize oil.

Another embodiment comprises cyclosporin A, Emulsifier 632/90%®, and Maize oil.

Another embodiment comprises cyclosporin A, Geronol AG-821®, and Maize oil.

Another embodiment comprises cyclosporin A, Gradonic N-95®, and Maize oil.

Another embodiment comprises cyclosporin A, Hetoxide NP-4®, and Maize oil.

Another embodiment comprises cyclosporin A, Hetoxide NP-30®, and Maize oil.

Another embodiment comprises cyclosporin A, Hostapal N-100®, and Maize oil.

Another embodiment comprises cyclosporin A, Hostapal N-110®, and Maize oil.

Another embodiment comprises cyclosporin A, Igepal CTA-639W®, and Maize oil.

Another embodiment comprises cyclosporin A, Igepal DAP-9®, and Maize oil.

Another embodiment comprises cyclosporin A, Igepal OD-410®, and Maize oil.

Another embodiment comprises cyclosporin A, Igepal SS-837®, and Maize oil.

Another embodiment comprises cyclosporin A, Lipocol NP-9 USP®, and Maize oil.

Another embodiment comprises cyclosporin A, Macol DNP-10®, and Maize oil.

Another embodiment comprises cyclosporin A, Marlophen NP 5®, and Maize oil.

Another embodiment comprises cyclosporin A, Marlophen P 1®, and Maize oil.

Another embodiment comprises cyclosporin A, Surfonic NB®, and Maize oil.

Another embodiment comprises cyclosporin A, Suronic OPB-307®, and Maize oil.

Another embodiment comprises cyclosporin A, Surfonic OPB-407®, and Maize oil.

Another embodiment comprises cyclosporin A, Syn Fac 334®, and Maize oil.

Another embodiment comprises cyclosporin A, Syn Fac 8216®, and Maize oil.

Another embodiment comprises cyclosporin A, Triton N-57®, and Maize oil.

Another embodiment comprises cyclosporin A, Trycol 6956®, and Maize oil.

Another embodiment comprises cyclosporin A, Trycol 6961®, and Maize oil.

Another embodiment comprises cyclosporin A, Trycol 6964®, and Maize oil.

Another embodiment comprises cyclosporin A, Trycol 6969®, and Maize oil.

Another embodiment comprises cyclosporin A, Trycol 6974®, and Maize oil.

Another embodiment comprises cyclosporin A, Witbreak DRB-127®, and Maize oil.

Another embodiment comprises cyclosporin A, Witbreak DRB-127®, and Maize oil.

Another embodiment comprises cyclosporin A, Witconol NP Series®, and Maize oil.

Another embodiment comprises cyclosporin A, an Ethoxylated Aryl Phenol, and Maize oil.

Another embodiment comprises cyclosporin A, Soprophor BSU®, and Maize oil.

Another embodiment comprises cyclosporin A, Soprophor CY/8®, and Maize oil.

Another embodiment comprises cyclosporin A, Soprophor S/25®, and Maize oil.

Another embodiment comprises cyclosporin A, Witconol NIO®, and Maize oil.

Another embodiment comprises cyclosporin A, Witconol NIW®, and Maize oil.

Another embodiment comprises cyclosporin A, Witconol S-100®, and Maize oil.

Another embodiment comprises cyclosporin A, an Ethoxylated Fatty Acid. and Maize oil.

Another embodiment comprises cyclosporin A, Aldo PGHMS®, and Maize oil.

Another embodiment comprises cyclosporin A, Alkamuls TO-15/HR®, and Maize oil.

Another embodiment comprises cyclosporin A, Armotan AL-69-66®, and Maize oil.

Another embodiment comprises cyclosporin A, Cerasynt 840®, and Maize oil.

Another embodiment comprises cyclosporin A, Cerasynt 945®, and Maize oil.

Another embodiment comprises cyclosporin A, Crystal Inhibitor No. 5®, and Maize oil.

Another embodiment comprises cyclosporin A, DeThox Acid L-9®, and Maize oil.

Another embodiment comprises cyclosporin A, DeThox Acid S-8®, and Maize oil.

Another embodiment comprises cyclosporin A, Ethofat 242/25®, and Maize oil.

Another embodiment comprises cyclosporin A, Hydropalat 65®, and Maize oil.

Another embodiment comprises cyclosporin A, Lipo EGMS®, and Maize oil.

Another embodiment comprises cyclosporin A, Lipopeg 2 DL®, and Maize oil.

Another embodiment comprises cyclosporin A, Lipopeg 4 DL®, and Maize oil.

Another embodiment comprises cyclosporin A, Lipopeg 4-L®, and Maize oil;

Another embodiment comprises cyclosporin A, Lipopeg 39-S®, and Maize oil.

Another embodiment comprises cyclosporin A, Lipopeg 4-S®, and Maize oil.

Another embodiment comprises cyclosporin A, Lipopeg 10-S®, and Maize oil.

Another embodiment comprises cyclosporin A, Lipopeg 100-S®, and Maize oil.

Another embodiment comprises cyclosporin A, Lipopeg 6000 DS®, and Maize oil.

Another embodiment comprises cyclosporin A, Lumulse 40-L®, and Maize oil.

Another embodiment comprises cyclosporin A, Lumulse 40-S®, and Maize oil.

Another embodiment comprises cyclosporin A, Lumulse 42-L®, and Maize oil.

Another embodiment comprises cyclosporin A, Lumulse 42-S®, and Maize oil.

Another embodiment comprises cyclosporin A, Lumulse 100-S®, and Maize oil.

Another embodiment comprises cyclosporin A, Lumulse 602-S®, and Maize oil.

Another embodiment comprises cyclosporin A, Magrabar PGE-20-0®, and Maize oil.

Another embodiment comprises cyclosporin A, Magrabar PGE-20L®, and Maize oil.

Another embodiment comprises cyclosporin A, Magrabar PGE-20T®, and Maize oil.

Another embodiment comprises cyclosporin A, Magrabar PGE-22-0®, and Maize oil.

Another embodiment comprises cyclosporin A, Magrabar PGE-22L®, and Maize oil.

Another embodiment comprises cyclosporin A, Magrabar PGE-22T®, and Maize oil.

Another embodiment comprises cyclosporin A, Magrabar PGE-40-0®, and Maize oil.

Another embodiment comprises cyclosporin A, Magrabar PGE-40L®, and Maize oil.

Another embodiment comprises cyclosporin A, Magrabar PGE-40T®, and Maize oil.

Another embodiment comprises cyclosporin A, Magrabar PGE-42-0®, and Maize oil.

Another embodiment comprises cyclosporin A, Magrabar PGE-42L®, and Maize oil.

Another embodiment comprises cyclosporin A, Magrabar PGE-42T®, and Maize oil.

Another embodiment comprises cyclosporin A, Magrabar PGE-60-0®, and Maize oil.

Another embodiment comprises cyclosporin A, Magrabar PGE-60L®, and Maize oil.

Another embodiment comprises cyclosporin A, Magrabar PGE-60T®, and Maize oil.

Another embodiment comprises cyclosporin A, Magrabar PGE-62-0®, and Maize oil.

Another embodiment comprises cyclosporin A, Magrabar PGE-62L®, and Maize oil.

Another embodiment comprises cyclosporin A, Magrabar PGE-62T®, and Maize oil.

Another embodiment comprises cyclosporin A, Mapeg S-40K®, and Maize oil.

Another embodiment comprises cyclosporin A, Marlowet OTS®, and Maize oil.

Another embodiment comprises cyclosporin A, Naturdchem PGR®, and Maize oil.

Another embodiment comprises cyclosporin A, PG No. 4®, and Maize oil.

Another embodiment comprises cyclosporin A, Renex 20®, and Maize oil.

Another embodiment comprises cyclosporin A, Ritox 52®, and Maize oil.

Another embodiment comprises cyclosporin A, Ritox 53®, and Maize oil.

Another embodiment comprises cyclosporin A, Ritox 59®, and Maize oil.

Another embodiment comprises cyclosporin A, Surfax 8916/A®, and Maize oil.

Another embodiment comprises cyclosporin A, Tego Acid S 40 P®, and Maize oil.

Another embodiment comprises cyclosporin A, Tego Acid S 100 P®, and Maize oil.

Another embodiment comprises cyclosporin A, Tween 20®, and Maize oil.

Another embodiment comprises cyclosporin A, Volpo 131®, and Maize oil.

Another embodiment comprises cyclosporin A, an Ethoxylated Fatty Ester or Oil. and Maize oil.

Another embodiment comprises cyclosporin A, Acconon 6-C10®, and Maize oil.

Another embodiment comprises cyclosporin A, Acconon CC-6®, and Maize oil.

Another embodiment comprises cyclosporin A, Acconon CO-7®, and Maize oil.

Another embodiment comprises cyclosporin A, Aldosperse 40/60 FG®, and Maize oil.

Another embodiment comprises cyclosporin A, Aldosperse ML-23®, and Maize oil.

Another embodiment comprises cyclosporin A, Aldosperse MS-20 FG®, and Maize oil.

Another embodiment comprises cyclosporin A, Alkamuls EL-620®, and Maize oil.

Another embodiment comprises cyclosporin A, Alkamuls EL-719®, and Maize oil.

Another embodiment comprises cyclosporin A, Alkamuls EL-985®, and Maize oil.

Another embodiment comprises cyclosporin A, Arlatone G®, and Maize oil.

Another embodiment comprises cyclosporin A, Arlatone T®, and Maize oil.

Another embodiment comprises cyclosporin A, Atlas G-1045A®, and Maize oil.

Another embodiment comprises cyclosporin A, Atlas G-1086®, and Maize oil.

Another embodiment comprises cyclosporin A, Atlas G-1087®, and Maize oil.

Another embodiment comprises cyclosporin A, Atlas G-1089®, and Maize oil.

Another embodiment comprises cyclosporin A, Atlas G-1096®, and Maize oil.

Another embodiment comprises cyclosporin A, Atlas G-1292®, and Maize oil.

Another embodiment comprises cyclosporin A, Atlas G-1293®, and Maize oil.

Another embodiment comprises cyclosporin A, Atlas G-1300®, and Maize oil.

Another embodiment comprises cyclosporin A, Atlas G-7076®, and Maize oil.

Another embodiment comprises cyclosporin A, Capmul EMG®, and Maize oil.

Another embodiment comprises cyclosporin A, Chemonic CO-40®, and Maize oil.

Another embodiment comprises cyclosporin A, Chemonic LI-3®, and Maize oil.

Another embodiment comprises cyclosporin A, Chemonic LI-7®, and Maize oil.

Another embodiment comprises cyclosporin A, Cirrasol GM®, and Maize oil.

Another embodiment comprises cyclosporin A, Cremophor CO 40®, and Maize oil.

Another embodiment comprises cyclosporin A, Cremophor CO 410®, and Maize oil.

Another embodiment comprises cyclosporin A, Cremophor EL®, and Maize oil.

Another embodiment comprises cyclosporin A, Cremophor GC7®, and Maize oil.

Another embodiment comprises cyclosporin A, Cremophor RH-40®, and Maize oil.

Another embodiment comprises cyclosporin A, Crovol A-40®, and Maize oil.

Another embodiment comprises cyclosporin A, Crovol A-70®, and Maize oil.

Another embodiment comprises cyclosporin A, Crovol M-70®, and Maize oil.

Another embodiment comprises cyclosporin A, Crovol PK-70®, and Maize oil.

Another embodiment comprises cyclosporin A, Cutina E-24®, and Maize oil.

Another embodiment comprises cyclosporin A, Dacospin 12-R®, and Maize oil.

Another embodiment comprises cyclosporin A, Dehymuls HRE-7®, and Maize oil.

Another embodiment comprises cyclosporin A, DeSonic 30C®, and Maize oil.

Another embodiment comprises cyclosporin A, DeSonic 36C®, and Maize oil.

Another embodiment comprises cyclosporin A, DeSonic 40C®, and Maize oil.

Another embodiment comprises cyclosporin A, Durfax 60®, and Maize oil.

Another embodiment comprises cyclosporin A, Durfax 65®, and Maize oil.

Another embodiment comprises cyclosporin A, Durfax 80®, and Maize oil.

Another embodiment comprises cyclosporin A, Durfax EOM®, and Maize oil.

Another embodiment comprises cyclosporin A, Eccoterge NF-2®, and Maize oil.

Another embodiment comprises cyclosporin A, Emulpon CO-360®, and Maize oil.

Another embodiment comprises cyclosporin A, Emulpon CO-550®, and Maize oil.

Another embodiment comprises cyclosporin A, Emulsogen EL®, and Maize oil.

Another embodiment comprises cyclosporin A, Emulsogen HCO 040®, and Maize oil.

Another embodiment comprises cyclosporin A, Emulsogen HCO 060®, and Maize oil.

Another embodiment comprises cyclosporin A, Emulsynt 1055®, and Maize oil.

Another embodiment comprises cyclosporin A, Ethox 3095®, and Maize oil.

Another embodiment comprises cyclosporin A, Eumulgin RO-40®, and Maize oil.

Another embodiment comprises cyclosporin A, Genapol G-260®, and Maize oil.

Another embodiment comprises cyclosporin A, Glycosperse L-20®, and Maize oil.

Another embodiment comprises cyclosporin A, Glycosperse O-5®, and Maize oil.

Another embodiment comprises cyclosporin A, Glycosperse O-20®, and Maize oil.

Another embodiment comprises cyclosporin A, Glycosperse O-20 FG®, and Maize oil.

Another embodiment comprises cyclosporin A, Glycosperse S-20®, and Maize oil.

Another embodiment comprises cyclosporin A, Glycosperse S-20 FG®, and Maize oil.

Another embodiment comprises cyclosporin A, Glycosperse TS-20®, and Maize oil.

Another embodiment comprises cyclosporin A, Glycosperse TS-20 FG®, and Maize oil.

Another embodiment comprises cyclosporin A, Hetan SL®, and Maize oil.

Another embodiment comprises cyclosporin A, Hetan SO®, and Maize oil.

Another embodiment comprises cyclosporin A, Hetan SS®, and Maize oil.

Another embodiment comprises cyclosporin A, Hetoxide C-2®, and Maize oil.

Another embodiment comprises cyclosporin A, Hetoxide C-9®, and Maize oil.

Another embodiment comprises cyclosporin A, Hetoxide C-15®, and Maize oil.

Another embodiment comprises cyclosporin A, Hetoxide C-25®, and Maize oil.

Another embodiment comprises cyclosporin A, Hetoxide C-40®, and Maize oil.

Another embodiment comprises cyclosporin A, Hetoxide C-200®, and Maize oil.

Another embodiment comprises cyclosporin A, Hetoxide C-200-50%®, and Maize oil.

Another embodiment comprises cyclosporin A, Hetoxide GC-30®, and Maize oil.

Another embodiment comprises cyclosporin A, Hetoxide HC-60®, and Maize oil.

Another embodiment comprises cyclosporin A, Ice No. 2®, and Maize oil.

Another embodiment comprises cyclosporin A, Incrocas 30/40®, and Maize oil.

Another embodiment comprises cyclosporin A, Lexol EC®, and Maize oil.

Another embodiment comprises cyclosporin A, Lexol EO®, and Maize oil.

Another embodiment comprises cyclosporin A, Lipocol HCO-40®, and Maize oil.

Another embodiment comprises cyclosporin A, Lipocol HCO-60®, and Maize oil.

Another embodiment comprises cyclosporin A, Lipocol O-3 Special®, and Maize oil.

Another embodiment comprises cyclosporin A, Lipopeg 2-L®, and Maize oil.

Another embodiment comprises cyclosporin A, Lipopeg 4-DO®, and Maize oil.

Another embodiment comprises cyclosporin A, Lipopeg 4-DS®, and Maize oil.

Another embodiment comprises cyclosporin A, Lipovol GTB®, and Maize oil.

Another embodiment comprises cyclosporin A, Lonzest SML-20®, and Maize oil.

Another embodiment comprises cyclosporin A, Lonzest SMO-20®, and Maize oil.

Another embodiment comprises cyclosporin A, Lonzest SMS-20®, and Maize oil.

Another embodiment comprises cyclosporin A, Lonzest STO-20®, and Maize oil.

Another embodiment comprises cyclosporin A, Lonzest STS-20®, and Maize oil.

Another embodiment comprises cyclosporin A, Lumulse GR-40®, and Maize oil.

Another embodiment comprises cyclosporin A, Lumulse GRH-40®, and Maize oil.

Another embodiment comprises cyclosporin A, Lumulse POE (7) GML®, and Maize oil.

Another embodiment comprises cyclosporin A, Lumulse POE (12) Glyc®, and Maize oil.

Another embodiment comprises cyclosporin A, Lumulse POE (40) MS KP®, and Maize oil.

Another embodiment comprises cyclosporin A, Marlowet 4750®, and Maize oil.

Another embodiment comprises cyclosporin A, Marlowet LVS®, and Maize oil.

Another embodiment comprises cyclosporin A, Marlowet R 11®, and Maize oil.

Another embodiment comprises cyclosporin A, Marlowet R 40®, and Maize oil.

Another embodiment comprises cyclosporin A, Mazol 80 MGK®, and Maize oil.

Another embodiment comprises cyclosporin A, Nonionic Emulsifier T-9®, and Maize oil.

Another embodiment comprises cyclosporin A, Oronal LOG®, and Maize oil.

Another embodiment comprises cyclosporin A, Polyderm PPI-CO-200®, and Maize oil.

Another embodiment comprises cyclosporin A, Polyderm PPI-CO-40®, and Maize oil.

Another embodiment comprises cyclosporin A, Rewoderm LI 520-70®, and Maize oil.

Another embodiment comprises cyclosporin A, Ritapeg 150 DS®, and Maize oil.

Another embodiment comprises cyclosporin A, Softigen 767®, and Maize oil.

Another embodiment comprises cyclosporin A, Surdactol 318®, and Maize oil.

Another embodiment comprises cyclosporin A, Surfactol 365®, and Maize oil.

Another embodiment comprises cyclosporin A, Syn Lube 107®, and Maize oil.

Another embodiment comprises cyclosporin A, Syn Lube 728®, and Maize oil.

Another embodiment comprises cyclosporin A, Syn Lube 1632H®, and Maize oil.

Another embodiment comprises cyclosporin A, Syn Lube 6277-A®, and Maize oil.

Another embodiment comprises cyclosporin A, T-Det C-20®, and Maize oil.

Another embodiment comprises cyclosporin A, T-Det C-40®, and Maize oil.

Another embodiment comprises cyclosporin A, Tally 100 Plus®, and Maize oil.

Another embodiment comprises cyclosporin A, Uniperol EL®, and Maize oil.

Another embodiment comprises cyclosporin A, a Fatty Ester, and Maize oil.

Another embodiment comprises cyclosporin A, Actralube-Syn 147®, and Maize oil.

Another embodiment comprises cyclosporin A, Atlas G-1556®, and Maize oil.

Another embodiment comprises cyclosporin A, Atlas G-1564®, and Maize oil.

Another embodiment comprises cyclosporin A, Atlasol Base Oil S®, and Maize oil.

Another embodiment comprises cyclosporin A, Base ML®, and Maize oil.

Another embodiment comprises cyclosporin A, Base MT®, and Maize oil.

Another embodiment comprises cyclosporin A, Cerasynt 303®, and Maize oil.

Another embodiment comprises cyclosporin A, Dermol 1012®, and Maize oil.

Another embodiment comprises cyclosporin A, Kemester 4000®, and Maize oil.

Another embodiment comprises cyclosporin A, Lactipol S®, and Maize oil.

Another embodiment comprises cyclosporin A, Magrabar PGO®, and Maize oil.

Another embodiment comprises cyclosporin A, Mayco Base BFO®, and Maize oil.

Another embodiment comprises cyclosporin A, Methyl Linoleate®, and Maize oil.

Another embodiment comprises cyclosporin A, Pationic 122A®, and Maize oil.

Another embodiment comprises cyclosporin A, Pationic 138C®, and Maize oil.

Another embodiment comprises cyclosporin A, Pationic CSL®, and Maize oil.

Another embodiment comprises cyclosporin A, Pationic ISL®, and Maize oil.

Another embodiment comprises cyclosporin A, Pationic SBL®, and Maize oil.

Another embodiment comprises cyclosporin A, Pationic SSL®, and Maize oil.

Another embodiment comprises cyclosporin A, Ritasol®, and Maize oil.

Another embodiment comprises cyclosporin A, Tego Alkanol CS 20®, and Maize oil.

Another embodiment comprises cyclosporin A, Tego Alkanol L23 P®, and Maize oil.

Another embodiment comprises cyclosporin A, Tego Alkanol S2®, and Maize oil.

Another embodiment comprises cyclosporin A, Tego Alkanol S20 P®, and Maize oil.

Another embodiment comprises cyclosporin A, Triemulsifier 600 MS®, and Maize oil.

Another embodiment comprises cyclosporin A, a Glycerol Ester®, and Maize oil.

Another embodiment comprises cyclosporin A, Agro #9 Wint SBO®, and Maize oil.

Another embodiment comprises cyclosporin A, Ahcovel Base 700®, and Maize oil.

Another embodiment comprises cyclosporin A, Aldo HMS FG®, and Maize oil.

Another embodiment comprises cyclosporin A, Aldo MLD®, and Maize oil.

Another embodiment comprises cyclosporin A, Aldo MLD FG®, and Maize oil.

Another embodiment comprises cyclosporin A, Aldo MO FG®, and Maize oil.

Another embodiment comprises cyclosporin A, Aldo MS®, and Maize oil.

Another embodiment comprises cyclosporin A, Aldo MS FG®, and Maize oil.

Another embodiment comprises cyclosporin A, Aldo MS LG FG®, and Maize oil.

Another embodiment comprises cyclosporin A, Aldo MSD®, and Maize oil.

Another embodiment comprises cyclosporin A, Aldo MSD FG®, and Maize oil.

Another embodiment comprises cyclosporin A, Aldosperse O-20 FG®, and Maize oil.

Another embodiment comprises cyclosporin A, Aldosperse TS-20 FG®, and Maize oil.

Another embodiment comprises cyclosporin A, Aldosperse TS-40 FG®, and Maize oil.

Another embodiment comprises cyclosporin A, Arlacel 165®, and Maize oil.

Another embodiment comprises cyclosporin A, Arlacel 186®, and Maize oil.

Another embodiment comprises cyclosporin A, Capmul GMO®, and Maize oil.

Another embodiment comprises cyclosporin A, Capmul GMS®, and Maize oil.

Another embodiment comprises cyclosporin A, Caprol 3GO®, and Maize oil.

Another embodiment comprises cyclosporin A, Caprol 3GVS®, and Maize oil.

Another embodiment comprises cyclosporin A, Caprol 6G2S®, and Maize oil.

Another embodiment comprises cyclosporin A, Caprol 10G40®, and Maize oil.

Another embodiment comprises cyclosporin A, Caprol 10G100®, and Maize oil.

Another embodiment comprises cyclosporin A, Caprol ET®, and Maize oil.

Another embodiment comprises cyclosporin A, Caprol PGE860®, and Maize oil.

Another embodiment comprises cyclosporin A, Cerasynt 945®, and Maize oil.

Another embodiment comprises cyclosporin A, Cerasynt GMS®, and Maize oil.

Another embodiment comprises cyclosporin A, Cerasynt Q®, and Maize oil.

Another embodiment comprises cyclosporin A, Cerasynt SD®, and Maize oil.

Another embodiment comprises cyclosporin A, Cerasynt WM®, and Maize oil.

Another embodiment comprises cyclosporin A, Chemsperse 14®, and Maize oil.

Another embodiment comprises cyclosporin A, Cremophor GO-32®, and Maize oil.

Another embodiment comprises cyclosporin A, Cremophor GS11®, and Maize oil.

Another embodiment comprises cyclosporin A, Cremophor GS-32®, and Maize oil.

Another embodiment comprises cyclosporin A, Cutina KD-16®, and Maize oil.

Another embodiment comprises cyclosporin A, Dehymuls PGPH®, and Maize oil.

Another embodiment comprises cyclosporin A, Dermol DGDIS®, and Maize oil.

Another embodiment comprises cyclosporin A, Dermol DGMIS®, and Maize oil.

Another embodiment comprises cyclosporin A, Dermol G-76®, and Maize oil.

Another embodiment comprises cyclosporin A, Dermol G-7DI®, and Maize oil.

Another embodiment comprises cyclosporin A, Dermol NGDI®, and Maize oil.

Another embodiment comprises cyclosporin A, Dermolan GLH®, and Maize oil.

Another embodiment comprises cyclosporin A, Drewmulse GMO®, and Maize oil.

Another embodiment comprises cyclosporin A, Drewpol 3-5-M®, and Maize oil.

Another embodiment comprises cyclosporin A, Durlac 100 W®, and Maize oil.

Another embodiment comprises cyclosporin A, Dur-Lo®, and Maize oil.

Another embodiment comprises cyclosporin A, Dynasan 118®, and Maize oil.

Another embodiment comprises cyclosporin A, EC-25®, and Maize oil.

Another embodiment comprises cyclosporin A, EM 40®, and Maize oil.

Another embodiment comprises cyclosporin A, Emerest 2400®, and Maize oil.

Another embodiment comprises cyclosporin A, Emerest 2452®, and Maize oil.

Another embodiment comprises cyclosporin A, Empilan G-26®, and Maize oil.

Another embodiment comprises cyclosporin A, Genapol TSM®, and Maize oil.

Another embodiment comprises cyclosporin A, Hostacerin DGI®, and Maize oil.

Another embodiment comprises cyclosporin A, Hostacerin DGL®, and Maize oil.

Another embodiment comprises cyclosporin A, Hostacerin DGMS®, and Maize oil.

Another embodiment comprises cyclosporin A, Hostacerin DGSB®, and Maize oil.

Another embodiment comprises cyclosporin A, Ice No. 2®, and Maize oil.

Another embodiment comprises cyclosporin A, lmwitor 742®, and Maize oil.

Another embodiment comprises cyclosporin A, lmwitor 780 K®, and Maize oil.

Another embodiment comprises cyclosporin A, Imwitor 960 Flakes®, and Maize oil.

Another embodiment comprises cyclosporin A, Isolan GI 34®, and Maize oil.

Another embodiment comprises cyclosporin A, Isolan GO 33®, and Maize oil.

Another embodiment comprises cyclosporin A, Kemester 1000®, and Maize oil.

Another embodiment comprises cyclosporin A, Kemester 2000®, and Maize oil.

Another embodiment comprises cyclosporin A, Kemester 2000®, and Maize oil.

Another embodiment comprises cyclosporin A, Kemester 6000SE®, and Maize oil.

Another embodiment comprises cyclosporin A, Lamecreme DGE 18®, and Maize oil.

Another embodiment comprises cyclosporin A, Lexemul 515®, and Maize oil.

Another embodiment comprises cyclosporin A, Lexemul 561®, and Maize oil.

Another embodiment comprises cyclosporin A, Lexemul AR®, and Maize oil.

Another embodiment comprises cyclosporin A, Lexemul AS®, and Maize oil.

Another embodiment comprises cyclosporin A, Lexemul GDL®, and Maize oil.

Another embodiment comprises cyclosporin A, Lexemul T®, and Maize oil.

Another embodiment comprises cyclosporin A, Lipomulse 165®, and Maize oil.

Another embodiment comprises cyclosporin A, Lumulse GML K®, and Maize oil.

Another embodiment comprises cyclosporin A, Lumulse GMO K®, and Maize oil.

Another embodiment comprises cyclosporin A, Lumulse GMR K®, and Maize oil.

Another embodiment comprises cyclosporin A, Lumulse GMT K®, and Maize oil.

Another embodiment comprises cyclosporin A, Magrabar GMC®, and Maize oil.

Another embodiment comprises cyclosporin A, Magrabar GMO-CK®, and Maize oil.

Another embodiment comprises cyclosporin A, Magrabar MDG-5050®, and Maize oil.

Another embodiment comprises cyclosporin A, Magrabar PGO-315®, and Maize oil.

Another embodiment comprises cyclosporin A, Magrabar PGO-1010®, and Maize oil.

Another embodiment comprises cyclosporin A, Mazol 300K®, and Maize oil.

Another embodiment comprises cyclosporin A, Mazol GMO-K®, and Maize oil.

Another embodiment comprises cyclosporin A, Mazol GMS-K®, and Maize oil.

Another embodiment comprises cyclosporin A, Mazol PG031-K®, and Maize oil.

Another embodiment comprises cyclosporin A, Miglyol 812®, and Maize oil.

Another embodiment comprises cyclosporin A, Norfox 165C®, and Maize oil.

Another embodiment comprises cyclosporin A, Schercemol GMIS®, and Maize oil.

Another embodiment comprises cyclosporin A, Tegin®, and Maize oil.

Another embodiment comprises cyclosporin A, Tegin 4100 Pellets®, and Maize oil.

Another embodiment comprises cyclosporin A, Tegin M Pellets®, and Maize oil.

Another embodiment comprises cyclosporin A, Tegin OV®, and Maize oil.

Another embodiment comprises cyclosporin A, Teginacid H®, and Maize oil.

Another embodiment comprises cyclosporin A, Tego Cosmo P813®, and Maize oil.

Another embodiment comprises cyclosporin A, Wickenol 535®, and Maize oil.

Another embodiment comprises cyclosporin A, Witconol 14®, and Maize oil.

Another embodiment comprises cyclosporin A, Witconol 14®, and Maize oil.

Another embodiment comprises cyclosporin A, Witconol 14F®, and Maize oil.

Another embodiment comprises cyclosporin A, Witconol 18L®, and Maize oil.

Another embodiment comprises cyclosporin A, Witconol GOT®, and Maize oil.

Another embodiment comprises cyclosporin A, Witconol MST®, and Maize oil.

Another embodiment comprises cyclosporin A, Witconol RHT®, and Maize oil.

Another embodiment comprises cyclosporin A, a Glycol Ester, and Maize oil.

Another embodiment comprises cyclosporin A, Alkamuls 600 DO®, and Maize oil.

Another embodiment comprises cyclosporin A, Alkamuls SEG®, and Maize oil.

Another embodiment comprises cyclosporin A, Atlas EM-2®, and Maize oil.

Another embodiment comprises cyclosporin A, Cerasynt IP®, and Maize oil.

Another embodiment comprises cyclosporin A, Cerasynt M®, and Maize oil.

Another embodiment comprises cyclosporin A, Cerasynt MN®, and Maize oil.

Another embodiment comprises cyclosporin A, Cerasynt PA®, and Maize oil.

Another embodiment comprises cyclosporin A, Chemsperse EGDS®, and Maize oil.

Another embodiment comprises cyclosporin A, Chemsperse EGMS®, and Maize oil.

Another embodiment comprises cyclosporin A, Colonial Monolaurin®, and Maize oil.

Another embodiment comprises cyclosporin A, DeMuls SGE-95®, and Maize oil.

Another embodiment comprises cyclosporin A, Eccoterge 200®, and Maize oil.

Another embodiment comprises cyclosporin A, Emerest 2380®, and Maize oil.

Another embodiment comprises cyclosporin A, Ethox 2610®, and Maize oil.

Another embodiment comprises cyclosporin A, Ethox DO-9®, and Maize oil.

Another embodiment comprises cyclosporin A, Ethox DO-14®, and Maize oil.

Another embodiment comprises cyclosporin A, Ethox SO-9®, and Maize oil.

Another embodiment comprises cyclosporin A, Fizul MD-318®, and Maize oil.

Another embodiment comprises cyclosporin A, Genapol EGDS-VHP®, and Maize oil.

Another embodiment comprises cyclosporin A, Genapol TS Powder®, and Maize oil.

Another embodiment comprises cyclosporin A, Hostacerin WO®, and Maize oil.

Another embodiment comprises cyclosporin A, Inversol 140®, and Maize oil.

Another embodiment comprises cyclosporin A, Kemester 104®, and Maize oil.

Another embodiment comprises cyclosporin A, Kemester 205®, and Maize oil.

Another embodiment comprises cyclosporin A, Kemester 226®, and Maize oil.

Another embodiment comprises cyclosporin A, Kemester 5221SE®, and Maize oil.

Another embodiment comprises cyclosporin A, Kemester EGDS®, and Maize oil.

Another embodiment comprises cyclosporin A, Lexemul EGDS®, and Maize oil.

Another embodiment comprises cyclosporin A, Lexemul EGMS®, and Maize oil.

Another embodiment comprises cyclosporin A, Lexemul P®, and Maize oil.

Another embodiment comprises cyclosporin A, Lipo DGLS®, and Maize oil.

Another embodiment comprises cyclosporin A, Lipo EGDS®, and Maize oil.

Another embodiment comprises cyclosporin A, Lipo PGMS®, and Maize oil.

Another embodiment comprises cyclosporin A, Liposorb S-4®, and Maize oil.

Another embodiment comprises cyclosporin A, Liposorb TO-20®, and Maize oil.

Another embodiment comprises cyclosporin A, Lumulse PGO®, and Maize oil.

Another embodiment comprises cyclosporin A, Mackester EGDS®, and Maize oil.

Another embodiment comprises cyclosporin A, Mackester EGMS®, and Maize oil.

Another embodiment comprises cyclosporin A, Mackester GSTP®, and Maize oil.

Another embodiment comprises cyclosporin A, Mackester Series®, and Maize oil.

Another embodiment comprises cyclosporin A, Magrabar PDG-50®, and Maize oil.

Another embodiment comprises cyclosporin A, Mapeg 6000 DS®, and Maize oil.

Another embodiment comprises cyclosporin A, Marlowet 4702®, and Maize oil.

Another embodiment comprises cyclosporin A, Monalube 305®, and Maize oil.

Another embodiment comprises cyclosporin A, Monalube 310®, and Maize oil.

Another embodiment comprises cyclosporin A, Monalube 315®, and Maize oil.

Another embodiment comprises cyclosporin A, Monalube 320®, and Maize oil.

Another embodiment comprises cyclosporin A, Monalube 325®, and Maize oil.

Another embodiment comprises cyclosporin A, Monalube 330®, and Maize oil.

Another embodiment comprises cyclosporin A, Naturechem PGHS®, and Maize oil.

Another embodiment comprises cyclosporin A, Polycastorol PLO-840®, and Maize oil.

Another embodiment comprises cyclosporin A, Polytex 10M®, and Maize oil.

Another embodiment comprises cyclosporin A, Ritasynt IP®, and Maize oil.

Another embodiment comprises cyclosporin A, Ross Chem PEG 600 DT®, and Maize oil.

Another embodiment comprises cyclosporin A, Schercemol PGMS®, and Maize oil.

Another embodiment comprises cyclosporin A, Sponto H-44C®, and Maize oil.

Another embodiment comprises cyclosporin A, Tegin G®, and Maize oil.

Another embodiment comprises cyclosporin A, Witbreak DGE-182®, and Maize oil.

Another embodiment comprises cyclosporin A, Witbreak DGE-182®, and Maize oil.

Another embodiment comprises cyclosporin A, Witbreak DRA-21®, and Maize oil.

Another embodiment comprises cyclosporin A, Witbreak DRA-21®, and Maize oil.

Another embodiment comprises cyclosporin A, Witbreak DRA-50®, and Maize oil.

Another embodiment comprises cyclosporin A, Witbreak DRA-50®, and Maize oil.

Another embodiment comprises cyclosporin A, Witconol F26-46®, and Maize oil.

Another embodiment comprises cyclosporin A, Witconol H-32®, and Maize oil.

Another embodiment comprises cyclosporin A, Witconol H-33®, and Maize oil.

Another embodiment comprises cyclosporin A, Witconol H-35A®, and Maize oil.

Another embodiment comprises cyclosporin A, Witconol RHP®, and Maize oil.

Another embodiment comprises cyclosporin A, a Lanolin-based Derivative, and Maize oil.

Another embodiment comprises cyclosporin A, Amerchol CAB®, and Maize oil.

Another embodiment comprises cyclosporin A, Amerchol L-101®, and Maize oil.

Another embodiment comprises cyclosporin A, Amerlate LFA-LO®, and Maize oil.

Another embodiment comprises cyclosporin A, Amerlate P®, and Maize oil.

Another embodiment comprises cyclosporin A, Barre Common Degras®, and Maize oil.

Another embodiment comprises cyclosporin A, Cholesterol NF®, and Maize oil.

Another embodiment comprises cyclosporin A, Crodalan AWS®, and Maize oil.

Another embodiment comprises cyclosporin A, Crodalan LA®, and Maize oil.

Another embodiment comprises cyclosporin A, Emery 1650®, and Maize oil.

Another embodiment comprises cyclosporin A, Emery 1650®, and Maize oil.

Another embodiment comprises cyclosporin A, Emery 1740®, and Maize oil.

Another embodiment comprises cyclosporin A, Emery 1740®, and Maize oil.

Another embodiment comprises cyclosporin A, Forlan 500®, and Maize oil.

Another embodiment comprises cyclosporin A, Forlan L®, and Maize oil.

Another embodiment comprises cyclosporin A, Laneto 50®, and Maize oil.

Another embodiment comprises cyclosporin A, Laneto 100®, and Maize oil.

Another embodiment comprises cyclosporin A, Laneto AWS®, and Maize oil.

Another embodiment comprises cyclosporin A, Lanfrax 1776®, and Maize oil.

Another embodiment comprises cyclosporin A, Lanfrax 1776®, and Maize oil.

Another embodiment comprises cyclosporin A, Lanogel 21®, and Maize oil.

Another embodiment comprises cyclosporin A, Lipolan®, and Maize oil.

Another embodiment comprises cyclosporin A, Lipolan 31®, and Maize oil.

Another embodiment comprises cyclosporin A, OHlan®, and Maize oil.

Another embodiment comprises cyclosporin A, Polychol 5®, and Maize oil.

Another embodiment comprises cyclosporin A, Polychol 15®, and Maize oil.

Another embodiment comprises cyclosporin A, Ritacetyl®, and Maize oil.

Another embodiment comprises cyclosporin A, Ritachol®, and Maize oil.

Another embodiment comprises cyclosporin A, Ritahydrox®, and Maize oil.

Another embodiment comprises cyclosporin A, Ritalafa®, and Maize oil.

Another embodiment comprises cyclosporin A, Ritalan®, and Maize oil.

Another embodiment comprises cyclosporin A, Ritalan AWS®, and Maize oil.

Another embodiment comprises cyclosporin A, Ritalan C®, and Maize oil.

Another embodiment comprises cyclosporin A, Ritawax®, and Maize oil.

Another embodiment comprises cyclosporin A, Ritawax AEO®, and Maize oil.

Another embodiment comprises cyclosporin A, Ritawax ALA®, and Maize oil.

Another embodiment comprises cyclosporin A, Solan/Solan 50/Super Solan®, and Maize oil.

Another embodiment comprises cyclosporin A, Super Hartolan/Hartolan®, and Maize oil.

Another embodiment comprises cyclosporin A, Supersat AWS-4®, and Maize oil.

Another embodiment comprises cyclosporin A, Supersat AWS-24®, and Maize oil.

Another embodiment comprises cyclosporin A, a Lecithin or Lecithin Derivative. and Maize oil.

Another embodiment comprises cyclosporin A, Alcolec®, and Maize oil.

Another embodiment comprises cyclosporin A, Lecithin®, and Maize oil.

Another embodiment comprises cyclosporin A, Lexin K®, and Maize oil.

Another embodiment comprises cyclosporin A, Natipide®, and Maize oil.

Another embodiment comprises cyclosporin A, a Liqnin or Lignin Derivative, and Maize oil.

Another embodiment comprises cyclosporin A, Diwatex XP 9®, and Maize oil.

Another embodiment comprises cyclosporin A, Dynasperse LCD®, and Maize oil.

Another embodiment comprises cyclosporin A, Indulin SAL®, and Maize oil.

Another embodiment comprises cyclosporin A, Indulin W-1®, and Maize oil.

Another embodiment comprises cyclosporin A, Indulin W-5®, and Maize oil.

Another embodiment comprises cyclosporin A, Lignosol FTA®, and Maize oil.

Another embodiment comprises cyclosporin A, Lignosol SFX-65®, and Maize oil.

Another embodiment comprises cyclosporin A, Marasperse 52 CP®, and Maize oil.

Another embodiment comprises cyclosporin A, Marasperse AG®, and Maize oil.

Another embodiment comprises cyclosporin A, Marasperse CBOS-4®, and Maize oil.

Another embodiment comprises cyclosporin A, Ufoxane 2®, and Maize oil.

Another embodiment comprises cyclosporin A, a Methyl Ester, and Maize oil.

Another embodiment comprises cyclosporin A, E.B. Cleaner AK®, and Maize oil.

Another embodiment comprises cyclosporin A, Oleocal ME-70®, and Maize oil.

Another embodiment comprises cyclosporin A, Trade Name Company®, and Maize oil.

Another embodiment comprises cyclosporin A, Methyl Esters (cont'd)®, and Maize oil.

Another embodiment comprises cyclosporin A, Oleocal ME-92®, and Maize oil.

Another embodiment comprises cyclosporin A, Oleocal ME-112®, and Maize oil.

Another embodiment comprises cyclosporin A, Oleocal ME-130®, and Maize oil.

Another embodiment comprises cyclosporin A, a Monocqlyceride or a Derivative thereof, and Maize oil.

Another embodiment comprises cyclosporin A, Dynacet 211®, and Maize oil.

Another embodiment comprises cyclosporin A, Hetsorb S-20®, and Maize oil.

Another embodiment comprises cyclosporin A, lmwitor 191®, and Maize oil.

Another embodiment comprises cyclosporin A, lmwitor 370®, and Maize oil.

Another embodiment comprises cyclosporin A, lmwitor 375®, and Maize oil.

Another embodiment comprises cyclosporin A, lmwitor 900®, and Maize oil.

Another embodiment comprises cyclosporin A, lmwitor 945®, and Maize oil.

Another embodiment comprises cyclosporin A, lmwitor 2020®, and Maize oil.

Another embodiment comprises cyclosporin A, Kemester 5500®, and Maize oil.

Another embodiment comprises cyclosporin A, Kemester 6000®, and Maize oil.

Another embodiment comprises cyclosporin A, Magrabar GMC®, and Maize oil.

Another embodiment comprises cyclosporin A, Magrabar GMO-CK®, and Maize oil.

Another embodiment comprises cyclosporin A, Magrabar GPC-10®, and Maize oil.

Another embodiment comprises cyclosporin A, Magrabar MDG-5050®, and Maize oil.

Another embodiment comprises cyclosporin A, Monalube 335®, and Maize oil.

Another embodiment comprises cyclosporin A, Monoglycerides Glyceryl Monestearate Archer Daniels Midland Company®, and Maize oil.

Another embodiment comprises cyclosporin A, Rita GMS®, and Maize oil.

Another embodiment comprises cyclosporin A, Ritamulse SCG®, and Maize oil.

Another embodiment comprises cyclosporin A, Softigen 701®, and Maize oil.

Another embodiment comprises cyclosporin A, Tally 100 Plus®, and Maize oil.

Another embodiment comprises cyclosporin A, a Polyethylene Glycol, and Maize oil.

Another embodiment comprises cyclosporin A, Emulgade PL 68/50®, and Maize oil.

Another embodiment comprises cyclosporin A, Lumulse PEG®, and Maize oil.

Another embodiment comprises cyclosporin A, Rhodasun PEG-400®, and Maize oil.

Another embodiment comprises cyclosporin A, Rhodasurf PEG-600®, and Maize oil.

Another embodiment comprises cyclosporin A, Witconol PEG-400®, and Maize oil.

Another embodiment comprises cyclosporin A, a Polymeric Surfactant. and Maize oil.

Another embodiment comprises cyclosporin A, Acritamer PNC-EG®, and Maize oil.

Another embodiment comprises cyclosporin A, Ag-Rho DEP-775®, and Maize oil.

Another embodiment comprises cyclosporin A, APG 325N Glycoside®, and Maize oil.

Another embodiment comprises cyclosporin A, Aristoflex AVC®, and Maize oil.

Another embodiment comprises cyclosporin A, Aristoflex HMB®, and Maize oil.

Another embodiment comprises cyclosporin A, Burco NPS-225®, and Maize oil.

Another embodiment comprises cyclosporin A, Burco NPS-816®, and Maize oil.

Another embodiment comprises cyclosporin A, Chemccinate 5603®, and Maize oil.

Another embodiment comprises cyclosporin A, Cosmedia Guar C-261N®, and Maize oil.

Another embodiment comprises cyclosporin A, Gantrez S-95®, and Maize oil.

Another embodiment comprises cyclosporin A, Glucopon 220 UP®, and Maize oil.

Another embodiment comprises cyclosporin A, Glucopon 225 DK®, and Maize oil.

Another embodiment comprises cyclosporin A, Glucopon 425 N®, and Maize oil.

Another embodiment comprises cyclosporin A, Glucopon 600 UP®, and Maize oil.

Another embodiment comprises cyclosporin A, Glucopon 625 UP®, and Maize oil.

Another embodiment comprises cyclosporin A, Pemulen 1621®, and Maize oil.

Another embodiment comprises cyclosporin A, Pemulen 1622®, and Maize oil.

Another embodiment comprises cyclosporin A, Pemulen TR-1®, and Maize oil.

Another embodiment comprises cyclosporin A, Pemulen TR-2®, and Maize oil.

Another embodiment comprises cyclosporin A, Plantacare 818®, and Maize oil.

Another embodiment comprises cyclosporin A, Plantapon LGC Sorb®, and Maize oil.

Another embodiment comprises cyclosporin A, Plantaren 1200N®, and Maize oil.

Another embodiment comprises cyclosporin A, Plantaren 2000N®, and Maize oil.

Another embodiment comprises cyclosporin A, Viscolam AT 64®, and Maize oil.

Another embodiment comprises cyclosporin A, Viscolam AT 64P®, and Maize oil.

Another embodiment comprises cyclosporin A, Viscolam AT 100®, and Maize oil.

Another embodiment comprises cyclosporin A, Viscolam MAC 7®, and Maize oil.

Another embodiment comprises cyclosporin A, Viscolam SMC 20®, and Maize oil.

Another embodiment comprises cyclosporin A, Witbreak RTC-323®, and Maize oil.

Another embodiment comprises cyclosporin A, WSI 3700®, and Maize oil.

Another embodiment comprises cyclosporin A, a Propoxylated or Ethoxylated Fatty Acid, Alcohol, or Alkyl Phenol, and Maize oil.

Another embodiment comprises cyclosporin A, Antarox AA-60®, and Maize oil.

Another embodiment comprises cyclosporin A, Antarox LF-224®, and Maize oil.

Another embodiment comprises cyclosporin A, Burcomul DFE-45®, and Maize oil.

Another embodiment comprises cyclosporin A, Burcoterge LFE-1000®, and Maize oil.

Another embodiment comprises cyclosporin A, Chemal LF-25B®, and Maize oil.

Another embodiment comprises cyclosporin A, Chemal LF-40B®, and Maize oil.

Another embodiment comprises cyclosporin A, Dehypon LS-36®, and Maize oil.

Another embodiment comprises cyclosporin A, Dehypon LS-36®, and Maize oil.

Another embodiment comprises cyclosporin A, Dehypon LS-54®, and Maize oil.

Another embodiment comprises cyclosporin A, Dehypon LS-54®, and Maize oil.

Another embodiment comprises cyclosporin A, Delonic 100 VLF®, and Maize oil.

Another embodiment comprises cyclosporin A, Delonic LF-60 MOD®, and Maize oil.

Another embodiment comprises cyclosporin A, Empiderm B®, and Maize oil.

Another embodiment comprises cyclosporin A, Ethylan 1206®, and Maize oil.

Another embodiment comprises cyclosporin A, Ethylan NS-500K®, and Maize oil.

Another embodiment comprises cyclosporin A, Ethylan NS-500LQ®, and Maize oil.

Another embodiment comprises cyclosporin A, Genapol 1392®, and Maize oil.

Another embodiment comprises cyclosporin A, Genapol 2317®, and Maize oil.

Another embodiment comprises cyclosporin A, Genapol 26EP710®, and Maize oil.

Another embodiment comprises cyclosporin A, Genapol EP 1022®, and Maize oil.

Another embodiment comprises cyclosporin A, Genapol EP 1024®, and Maize oil.

Another embodiment comprises cyclosporin A, Genapol EP 6068®, and Maize oil.

Another embodiment comprises cyclosporin A, Genapol NP915®, and Maize oil.

Another embodiment comprises cyclosporin A, Kieralon MFB®, and Maize oil.

Another embodiment comprises cyclosporin A, Lumisolve CSA-80 V®, and Maize oil.

Another embodiment comprises cyclosporin A, Marlowet 5001®, and Maize oil.

Another embodiment comprises cyclosporin A, Marlox FK 64®, and Maize oil.

Another embodiment comprises cyclosporin A, Marlox MO 124®, and Maize oil.

Another embodiment comprises cyclosporin A, Marlox S 58®, and Maize oil.

Another embodiment comprises cyclosporin A, Nonatell 1003®, and Maize oil.

Another embodiment comprises cyclosporin A, Nonatell 1038®, and Maize oil.

Another embodiment comprises cyclosporin A, Nonatell 1052®, and Maize oil.

Another embodiment comprises cyclosporin A, Nonatell 1061®, and Maize oil.

Another embodiment comprises cyclosporin A, Nonatell 1075®, and Maize oil.

Another embodiment comprises cyclosporin A, Nonatell 1088®, and Maize oil.

Another embodiment comprises cyclosporin A, Nonatell 1123®, and Maize oil.

Another embodiment comprises cyclosporin A, Nonatell 1153®, and Maize oil.

Another embodiment comprises cyclosporin A, Nonatell 1161®, and Maize oil.

Another embodiment comprises cyclosporin A, Nonatell 1172®, and Maize oil.

Another embodiment comprises cyclosporin A, Nonatell 1181®, and Maize oil.

Another embodiment comprises cyclosporin A, Norfox 36®, and Maize oil.

Another embodiment comprises cyclosporin A, Procetyl AWS®, and Maize oil.

Another embodiment comprises cyclosporin A, Sandoxylate SX 412®, and Maize oil.

Another embodiment comprises cyclosporin A, Sandoxylate SX 418®, and Maize oil.

Another embodiment comprises cyclosporin A, Surfonic JL-80X®, and Maize oil.

Another embodiment comprises cyclosporin A, Surfonic JL-80X-B1®, and Maize oil.

Another embodiment comprises cyclosporin A, Surfonic L4-29X®, and Maize oil.

Another embodiment comprises cyclosporin A, Surfonic LF®, and Maize oil.

Another embodiment comprises cyclosporin A, T-Det A826®, and Maize oil.

Another embodiment comprises cyclosporin A, T-Det LF-416®, and Maize oil.

Another embodiment comprises cyclosporin A, Tergitol Min-Foam 1X®, and Maize oil.

Another embodiment comprises cyclosporin A, Tergitol Min-Foam 2X®, and Maize oil.

Another embodiment comprises cyclosporin A, Triton CF-21®, and Maize oil.

Another embodiment comprises cyclosporin A, Triton CF-76®, and Maize oil.

Another embodiment comprises cyclosporin A, Triton XL-80N®, and Maize oil.

Another embodiment comprises cyclosporin A, Witconol NS-98®, and Maize oil.

Another embodiment comprises cyclosporin A, Witconol NS-108LQ®, and Maize oil.

Another embodiment comprises cyclosporin A, Witconol NS-145®, and Maize oil.

Another embodiment comprises cyclosporin A, Witconol NS-179®, and Maize oil.

Another embodiment comprises cyclosporin A, a Protein-based Surfactant, and Maize oil.

Another embodiment comprises cyclosporin A, AminoFoam W®, and Maize oil.

Another embodiment comprises cyclosporin A, Amiter LGOD-2®, and Maize oil.

Another embodiment comprises cyclosporin A, Amiter LGS-2®, and Maize oil.

Another embodiment comprises cyclosporin A, Amiter LGS-5®, and Maize oil.

Another embodiment comprises cyclosporin A, Lamepon S®, and Maize oil.

Another embodiment comprises cyclosporin A, Lamepon S®, and Maize oil.

Another embodiment comprises cyclosporin A, Maypon 4C®, and Maize oil.

Another embodiment comprises cyclosporin A, May-Tein C®, and Maize oil.

Another embodiment comprises cyclosporin A, May-Tein CT®, and Maize oil.

Another embodiment comprises cyclosporin A, May-Tein KTS®, and Maize oil.

Another embodiment comprises cyclosporin A, May-Tein SY®, and Maize oil.

Another embodiment comprises cyclosporin A, Plantapon S®, and Maize oil.

Another embodiment comprises cyclosporin A, Proteol APL®, and Maize oil.

Another embodiment comprises cyclosporin A, Proteol OAT®, and Maize oil.

Another embodiment comprises cyclosporin A, Pyroter CPI-40®, and Maize oil.

Another embodiment comprises cyclosporin A, Pyroter GPI-25®, and Maize oil.

Another embodiment comprises cyclosporin A, Supro-Tein S®, and Maize oil.

Another embodiment comprises cyclosporin A, Supro-Tein V®, and Maize oil.

Another embodiment comprises cyclosporin A,® and Maize oil.

Another embodiment comprises cyclosporin A, a Sarcosine Derivative®, and Maize oil.

Another embodiment comprises cyclosporin A, Crodasinic LS-30®, and Maize oil.

Another embodiment comprises cyclosporin A, Vanseal CS®, and Maize oil.

Another embodiment comprises cyclosporin A, Vanseal LS®, and Maize oil.

Another embodiment comprises cyclosporin A, Vanseal MS®, and Maize oil.

Another embodiment comprises cyclosporin A, Vanseal NACS-30®, and Maize oil.

Another embodiment comprises cyclosporin A, Vanseal NALS-95®, and Maize oil.

Another embodiment comprises cyclosporin A, Vanseal OS®, and Maize oil.

Another embodiment comprises cyclosporin A,® and Maize oil.

Another embodiment comprises cyclosporin A, a Silicone-based Surfactante®, and Maize oil.

Another embodiment comprises cyclosporin A, Abil-B-9950®, and Maize oil.

Another embodiment comprises cyclosporin A, Abil Care 85®, and Maize oil.

Another embodiment comprises cyclosporin A, Abil EM 90®, and Maize oil.

Another embodiment comprises cyclosporin A, Abil EM 97®, and Maize oil.

Another embodiment comprises cyclosporin A, Abil WE-09®, and Maize oil.

Another embodiment comprises cyclosporin A, Dow Corning 1248 Fluid®, and Maize oil.

Another embodiment comprises cyclosporin A, Dow Corning 3225C®, and Maize oil.

Another embodiment comprises cyclosporin A, Dow Corning 5200®, and Maize oil.

Another embodiment comprises cyclosporin A, Dow Corning Q4-3667®, and Maize oil.

Another embodiment comprises cyclosporin A, Monasil PCA®, and Maize oil.

Another embodiment comprises cyclosporin A, Monasil PDM®, and Maize oil.

Another embodiment comprises cyclosporin A, Monasil PLN®, and Maize oil.

Another embodiment comprises cyclosporin A, Polyderm PPI-SI-WS®, and Maize oil.

Another embodiment comprises cyclosporin A, Troysol 380W®, and Maize oil.

Another embodiment comprises cyclosporin A, Troysol S366®, and Maize oil.

Another embodiment comprises cyclosporin A, a Sorbitan Derivative, and Maize oil.

Another embodiment comprises cyclosporin A, Alkamuls SML®, and Maize oil.

Another embodiment comprises cyclosporin A, Alkamuls SMO®, and Maize oil.

Another embodiment comprises cyclosporin A, Alkamuls STO®, and Maize oil.

Another embodiment comprises cyclosporin A, Arlacel 20®, and Maize oil.

Another embodiment comprises cyclosporin A, Arlacel 40®, and Maize oil.

Another embodiment comprises cyclosporin A, Arlacel 60®, and Maize oil.

Another embodiment comprises cyclosporin A, Arlacel 80®, and Maize oil.

Another embodiment comprises cyclosporin A, Arlacel C®, and Maize oil.

Another embodiment comprises cyclosporin A, Armul 21®, and Maize oil.

Another embodiment comprises cyclosporin A, Atlox 80®, and Maize oil.

Another embodiment comprises cyclosporin A, Atlox 847®, and Maize oil.

Another embodiment comprises cyclosporin A, Atlox 1045A®, and Maize oil.

Another embodiment comprises cyclosporin A, Canarcel 20®, and Maize oil.

Another embodiment comprises cyclosporin A, Canarcel 60®, and Maize oil.

Another embodiment comprises cyclosporin A, Canarcel 80®, and Maize oil.

Another embodiment comprises cyclosporin A, Canarcel TW 20®, and Maize oil.

Another embodiment comprises cyclosporin A, Canarcel TW 60®, and Maize oil.

Another embodiment comprises cyclosporin A, Canarcel TW 80®, and Maize oil.

Another embodiment comprises cyclosporin A, Coladet BSB-P®, and Maize oil.

Another embodiment comprises cyclosporin A, Customulse O-20®, and Maize oil.

Another embodiment comprises cyclosporin A, Dehymuls E®, and Maize oil.

Another embodiment comprises cyclosporin A, DeSotan SMO®, and Maize oil.

Another embodiment comprises cyclosporin A, DeSotan SMO-20®, and Maize oil.

Another embodiment comprises cyclosporin A, DeSotan SMT®, and Maize oil.

Another embodiment comprises cyclosporin A, DeSotan SMT-20®, and Maize oil.

Another embodiment comprises cyclosporin A, Durfax 60®, and Maize oil.

Another embodiment comprises cyclosporin A, Durfax 65®, and Maize oil.

Another embodiment comprises cyclosporin A, Durfax 80®, and Maize oil.

Another embodiment comprises cyclosporin A, Durtan 60®, and Maize oil.

Another embodiment comprises cyclosporin A, Durtan 65®, and Maize oil.

Another embodiment comprises cyclosporin A, Liposorb L®, and Maize oil.

Another embodiment comprises cyclosporin A, Liposorb L-10®, and Maize oil.

Another embodiment comprises cyclosporin A, Liposorb L-20®, and Maize oil.

Another embodiment comprises cyclosporin A, Liposorb O®, and Maize oil.

Another embodiment comprises cyclosporin A, Liposorb O-20®, and Maize oil.

Another embodiment comprises cyclosporin A, Liposorb P®, and Maize oil.

Another embodiment comprises cyclosporin A, Liposorb P-20®, and Maize oil.

Another embodiment comprises cyclosporin A, Liposorb S®, and Maize oil.

Another embodiment comprises cyclosporin A, Liposorb S-20®, and Maize oil.

Another embodiment comprises cyclosporin A, Liposorb SQO®, and Maize oil.

Another embodiment comprises cyclosporin A, Liposorb TO®, and Maize oil.

Another embodiment comprises cyclosporin A, Liposorb TS®, and Maize oil.

Another embodiment comprises cyclosporin A, Liposorb TS-20®, and Maize oil.

Another embodiment comprises cyclosporin A, Lumisorb PS®, and Maize oil.

Another embodiment comprises cyclosporin A, Lumisorb SMO (T)®, and Maize oil.

Another embodiment comprises cyclosporin A, Lumisorb SMS K®, and Maize oil.

Another embodiment comprises cyclosporin A, Lumisorb SSO®, and Maize oil.

Another embodiment comprises cyclosporin A, Lumisorb STS K®, and Maize oil.

Another embodiment comprises cyclosporin A, Lumisorb STT®, and Maize oil.

Another embodiment comprises cyclosporin A, Magrabar SMO®, and Maize oil.

Another embodiment comprises cyclosporin A, Magrabar SMO-VEG®, and Maize oil.

Another embodiment comprises cyclosporin A, Magrabar SMT®, and Maize oil.

Another embodiment comprises cyclosporin A, Magrabar STO®, and Maize oil.

Another embodiment comprises cyclosporin A, Miracare BC-27®, and Maize oil.

Another embodiment comprises cyclosporin A, Ritabate 20®, and Maize oil.

Another embodiment comprises cyclosporin A, Ritabate 40®, and Maize oil.

Another embodiment comprises cyclosporin A, Ritabate 60®, and Maize oil.

Another embodiment comprises cyclosporin A, Ritabate 80®, and Maize oil.

Another embodiment comprises cyclosporin A, T-Maz®, and Maize oil.

Another embodiment comprises cyclosporin A, Tego SML®, and Maize oil.

Another embodiment comprises cyclosporin A, Tego SML 20®, and Maize oil.

Another embodiment comprises cyclosporin A, Tego SMO 80 V®, and Maize oil.

Another embodiment comprises cyclosporin A, Tego SMO V®, and Maize oil.

Another embodiment comprises cyclosporin A, Tego SMS®, and Maize oil.

Another embodiment comprises cyclosporin A, Tego STO V®, and Maize oil.

Another embodiment comprises cyclosporin A, Tween 21®, and Maize oil.

Another embodiment comprises cyclosporin A, Tween 40®, and Maize oil.

Another embodiment comprises cyclosporin A, Tween 60®, and Maize oil.

Another embodiment comprises cyclosporin A, Tween 60 K®, and Maize oil.

Another embodiment comprises cyclosporin A, Tween 61®, and Maize oil.

Another embodiment comprises cyclosporin A, Tween 65®, and Maize oil.

Another embodiment comprises cyclosporin A, Tween 80®, and Maize oil.

Another embodiment comprises cyclosporin A, Tween 80 K®, and Maize oil.

Another embodiment comprises cyclosporin A, Tween 81®, and Maize oil.

Another embodiment comprises cyclosporin A, Tween 85®, and Maize oil.

Another embodiment comprises cyclosporin A, a Sucrose or Glucose Ester, or Derivative thereof, and Maize oil.

Another embodiment comprises cyclosporin A, DeSulf GOS-P-60WCG®, and Maize oil.

Another embodiment comprises cyclosporin A, Glucam E-20 Distearate®, and Maize oil.

Another embodiment comprises cyclosporin A, Glucamate DOE-120®, and Maize oil.

Another embodiment comprises cyclosporin A, Glucamate SSE-20®, and Maize oil.

Another embodiment comprises cyclosporin A, Glucate DO®, and Maize oil.

Another embodiment comprises cyclosporin A, Glucate SS®, and Maize oil.

Another embodiment comprises cyclosporin A, Glucopon 425 UP®, and Maize oil.

Another embodiment comprises cyclosporin A, Isolan IS®, and Maize oil.

Another embodiment comprises cyclosporin A, Mazon 40®, and Maize oil.

Another embodiment comprises cyclosporin A, Montanov 82®, and Maize oil.

Another embodiment comprises cyclosporin A, Montanov 202®, and Maize oil.

Another embodiment comprises cyclosporin A, Montanov S®, and Maize oil.

Another embodiment comprises cyclosporin A, Rheozan®, and Maize oil.

Another embodiment comprises cyclosporin A, Simulsol AS 48®, and Maize oil.

Another embodiment comprises cyclosporin A, Simulsol SL 4®, and Maize oil.

Another embodiment comprises cyclosporin A, Simulsol SL 10®, and Maize oil.

Another embodiment comprises cyclosporin A, Simulsol SL 11V®, and Maize oil.

Another embodiment comprises cyclosporin A, Simulsol SL 55®, and Maize oil.

Another embodiment comprises cyclosporin A, Suga Nate 100 and 160®, and Maize oil.

Another embodiment comprises cyclosporin A, Tego Care 450®, and Maize oil.

Another embodiment comprises cyclosporin A, Tego Care CG 90®, and Maize oil.

Another embodiment comprises cyclosporin A, Tego Care PS®, and Maize oil.

Another embodiment comprises cyclosporin A, Tegosoft PSE 141 G®, and Maize oil.

Another embodiment comprises cyclosporin A, Tegotens G 826®, and Maize oil.

Another embodiment comprises cyclosporin A, Triton BG-10 (70%)®, and Maize oil.

Another embodiment comprises cyclosporin A, Triton CG-110 (60%)®, and Maize oil.

Another embodiment comprises cyclosporin A, Wickenol 545®, and Maize oil.

Another embodiment comprises cyclosporin A, an alcohol, and Linseed oil.

Another embodiment comprises cyclosporin A, Diglycerol®, and Linseed oil.

Another embodiment comprises cyclosporin A, Hetoxide GT-80®, and Linseed oil.

Another embodiment comprises cyclosporin A, Lexemul BEO®, and Linseed oil.

Another embodiment comprises cyclosporin A, Polyglycerol-3®, and Linseed oil.

Another embodiment comprises cyclosporin A, Redicote E Series®, and Linseed oil.

Another embodiment comprises cyclosporin A, Simulsol OX 1005L®, and Linseed oil.

Another embodiment comprises cyclosporin A, Stanfax 567®, and Linseed oil.

Another embodiment comprises cyclosporin A, TA-1618®, and Linseed oil.

Another embodiment comprises cyclosporin A, Witconol H-31A®, and Linseed oil.

Another embodiment comprises cyclosporin A, an amine oxide, and Linseed oil.

Another embodiment comprises cyclosporin A, AO-405®, and Linseed oil.

Another embodiment comprises cyclosporin A, AO-455®, and Linseed oil.

Another embodiment comprises cyclosporin A, AO 728 Special®, and Linseed oil.

Another embodiment comprises cyclosporin A, Barlox 12®, and Linseed oil.

Another embodiment comprises cyclosporin A, Barlox 14®, and Linseed oil.

Another embodiment comprises cyclosporin A, Burcoxide Lo®, and Linseed oil.

Another embodiment comprises cyclosporin A, Caloxamine LO®, and Linseed oil.

Another embodiment comprises cyclosporin A, Chemoxide CAW®, and Linseed oil.

Another embodiment comprises cyclosporin A, Chemoxide LM-30®, and Linseed oil.

Another embodiment comprises cyclosporin A, Chemoxide LO®, and Linseed oil.

Another embodiment comprises cyclosporin A, Chemoxide MO®, and Linseed oil.

Another embodiment comprises cyclosporin A, Colalux CAO-35®, and Linseed oil.

Another embodiment comprises cyclosporin A, Colalux LO®, and Linseed oil.

Another embodiment comprises cyclosporin A, DeMox CAPO®, and Linseed oil.

Another embodiment comprises cyclosporin A, DeMox CSG-30®, and Linseed oil.

Another embodiment comprises cyclosporin A, DeMox LAO®, and Linseed oil.

Another embodiment comprises cyclosporin A, Emcol L®, and Linseed oil.

Another embodiment comprises cyclosporin A, Empigen OB®, and Linseed oil.

Another embodiment comprises cyclosporin A, Empigen OS/A®, and Linseed oil.

Another embodiment comprises cyclosporin A, Foamox CDO®, and Linseed oil.

Another embodiment comprises cyclosporin A, Foamox DMM®, and Linseed oil.

Another embodiment comprises cyclosporin A, Foamox DMS®, and Linseed oil.

Another embodiment comprises cyclosporin A, Genaminox KC®, and Linseed oil.

Another embodiment comprises cyclosporin A, Genaminox LA®, and Linseed oil.

Another embodiment comprises cyclosporin A, Hartofoam SAO®, and Linseed oil.

Another embodiment comprises cyclosporin A, Hartox DMCD®, and Linseed oil.

Another embodiment comprises cyclosporin A, Lipowax DAT®, and Linseed oil.

Another embodiment comprises cyclosporin A, Lipowax PB Pastilles®, and Linseed oil.

Another embodiment comprises cyclosporin A, Mackamine C8®, and Linseed oil.

Another embodiment comprises cyclosporin A, Mackamine C10®, and Linseed oil.

Another embodiment comprises cyclosporin A, Mackamine C14®, and Linseed oil.

Another embodiment comprises cyclosporin A, Mackamine CAO®, and Linseed oil.

Another embodiment comprises cyclosporin A, Mackamine CO®, and Linseed oil.

Another embodiment comprises cyclosporin A, Mackamine LO®, and Linseed oil.

Another embodiment comprises cyclosporin A, Mackamine O2®, and Linseed oil.

Another embodiment comprises cyclosporin A, Mackamine SAO®, and Linseed oil.

Another embodiment comprises cyclosporin A, Mackamine SO®, and Linseed oil.

Another embodiment comprises cyclosporin A, Mazox KCAO®, and Linseed oil.

Another embodiment comprises cyclosporin A, Monalac MO®, and Linseed oil.

Another embodiment comprises cyclosporin A, Norfox LDA®, and Linseed oil.

Another embodiment comprises cyclosporin A, Rhodamox LO®, and Linseed oil.

Another embodiment comprises cyclosporin A, Schercamox C-AA®, and Linseed oil.

Another embodiment comprises cyclosporin A, Schercamox DMA®, and Linseed oil.

Another embodiment comprises cyclosporin A, Schercamox DML®, and Linseed oil.

Another embodiment comprises cyclosporin A, Schercamox DMM®, and Linseed oil.

Another embodiment comprises cyclosporin A, Schercamox DMS®, and Linseed oil.

Another embodiment comprises cyclosporin A, Tegotens DO®, and Linseed oil.

Another embodiment comprises cyclosporin A, Tomah AO-14-2®, and Linseed oil.

Another embodiment comprises cyclosporin A, Triaminox CDO®, and Linseed oil.

Another embodiment comprises cyclosporin A, a block polymer, and Linseed oil.

Another embodiment comprises cyclosporin A, AL 2070®, and Linseed oil.

Another embodiment comprises cyclosporin A, Antarox 17-R-2®, and Linseed oil.

Another embodiment comprises cyclosporin A, Antarox 25-R-2®, and Linseed oil.

Another embodiment comprises cyclosporin A, Antarox 31-R-1®, and Linseed oil.

Another embodiment comprises cyclosporin A, Antarox P-84®, and Linseed oil.

Another embodiment comprises cyclosporin A, Antarox P-104/H®, and Linseed oil.

Another embodiment comprises cyclosporin A, Arnox BP-Series®, and Linseed oil.

Another embodiment comprises cyclosporin A, Chemonic 435®, and Linseed oil.

Another embodiment comprises cyclosporin A, Chemonic D-25®, and Linseed oil.

Another embodiment comprises cyclosporin A, Chemonic PL Series®, and Linseed oil.

Another embodiment comprises cyclosporin A, Ethox L-121®, and Linseed oil.

Another embodiment comprises cyclosporin A, Ethox L-122®, and Linseed oil.

Another embodiment comprises cyclosporin A, Genapol PF-10®, and Linseed oil.

Another embodiment comprises cyclosporin A, Genapol PF-20®, and Linseed oil.

Another embodiment comprises cyclosporin A, Genapol PF-40A®, and Linseed oil.

Another embodiment comprises cyclosporin A, Norfox 2-LF®, and Linseed oil.

Another embodiment comprises cyclosporin A, Simulsol NW 342®, and Linseed oil.

Another embodiment comprises cyclosporin A, T-Det BP-1®, and Linseed oil.

Another embodiment comprises cyclosporin A, T-Det XD®, and Linseed oil.

Another embodiment comprises cyclosporin A, T-Det XH®, and Linseed oil.

Another embodiment comprises cyclosporin A, Triton CF-32®, and Linseed oil.

Another embodiment comprises cyclosporin A, Witconol 171®, and Linseed oil.

Another embodiment comprises cyclosporin A, Witconol 324®, and Linseed oil.

Another embodiment comprises cyclosporin A, Witconol 324D®, and Linseed oil.

Another embodiment comprises cyclosporin A, Witconol PD-2000®, and Linseed oil.

Another embodiment comprises cyclosporin A, a Carboxylated Alcohol or Alkylphenol Ethoxylate, and Linseed oil.

Another embodiment comprises cyclosporin A, Emcol CN-6®, and Linseed oil.

Another embodiment comprises cyclosporin A, Ethcarb®, and Linseed oil.

Another embodiment comprises cyclosporin A, Gemtex WNT-Conc®, and Linseed oil.

Another embodiment comprises cyclosporin A, Incrodet TD7-C®, and Linseed oil.

Another embodiment comprises cyclosporin A, Marlinat CM 105/80®, and Linseed oil.

Another embodiment comprises cyclosporin A, Marlovwet 1072®, and Linseed oil.

Another embodiment comprises cyclosporin A, Marlowet 4530®, and Linseed oil.

Another embodiment comprises cyclosporin A, Marlowet 4530LF®, and Linseed oil.

Another embodiment comprises cyclosporin A, Marlowet 4534®, and Linseed oil.

Another embodiment comprises cyclosporin A, Marlowet 4538®, and Linseed oil.

Another embodiment comprises cyclosporin A, Marlowet 4539®, and Linseed oil.

Another embodiment comprises cyclosporin A, Marlowet 4539LF®, and Linseed oil.

Another embodiment comprises cyclosporin A, Marlowet 4541®, and Linseed oil.

Another embodiment comprises cyclosporin A, Miranate LEC-80®, and Linseed oil.

Another embodiment comprises cyclosporin A, Sandopan B®, and Linseed oil.

Another embodiment comprises cyclosporin A, Sandopan B Modified®, and Linseed oil.

Another embodiment comprises cyclosporin A, Sandopan LS-24 Gel®, and Linseed oil.

Another embodiment comprises cyclosporin A, Surfine T-A®, and Linseed oil.

Another embodiment comprises cyclosporin A, a Carboxvlic Acid/Fatty Acid. and Linseed oil.

Another embodiment comprises cyclosporin A, Colaterge RAM®, and Linseed oil.

Another embodiment comprises cyclosporin A, Colatrope INC®, and Linseed oil.

Another embodiment comprises cyclosporin A, Crodacid B®, and Linseed oil.

Another embodiment comprises cyclosporin A, DeTrope CA-100®, and Linseed oil.

Another embodiment comprises cyclosporin A, Latol MTO®, and Linseed oil.

Another embodiment comprises cyclosporin A, Lumulse CC-33 K®, and Linseed oil.

Another embodiment comprises cyclosporin A, Mulls 2218®, and Linseed oil.

Another embodiment comprises cyclosporin A, OL-600®, and Linseed oil.

Another embodiment comprises cyclosporin A, OL-800®, and Linseed oil.

Another embodiment comprises cyclosporin A, R-910®, and Linseed oil.

Another embodiment comprises cyclosporin A, S-210®, and Linseed oil.

Another embodiment comprises cyclosporin A, Sandopan DTC Acid®, and Linseed oil.

Another embodiment comprises cyclosporin A, Sandopan LS 24 N®, and Linseed oil.

Another embodiment comprises cyclosporin A, Sandopan MA-18®, and Linseed oil.

Another embodiment comprises cyclosporin A, an Ethoxylated Alcohol, and Linseed oil.

Another embodiment comprises cyclosporin A, Adsee 799®, and Linseed oil.

Another embodiment comprises cyclosporin A, Adsee 799®, and Linseed oil.

Another embodiment comprises cyclosporin A, Alfonic 610-3.5®, and Linseed oil.

Another embodiment comprises cyclosporin A, Alfonic 810-2®, and Linseed oil.

Another embodiment comprises cyclosporin A, Alfonic 810-6®, and Linseed oil.

Another embodiment comprises cyclosporin A, Alfonic 1012-3®, and Linseed oil.

Another embodiment comprises cyclosporin A, Alfonic 1012-5®, and Linseed oil.

Another embodiment comprises cyclosporin A, Alfonic 1216CO-1.5®, and Linseed oil.

Another embodiment comprises cyclosporin A, Alfonic 1216CO-7®, and Linseed oil.

Another embodiment comprises cyclosporin A, Alfonic 1412-3®, and Linseed oil.

Another embodiment comprises cyclosporin A, Alfonic 1412-7®, and Linseed oil.

Another embodiment comprises cyclosporin A, Arlasolve 200®, and Linseed oil.

Another embodiment comprises cyclosporin A, Arlasolve 200 Liquid®, and Linseed oil.

Another embodiment comprises cyclosporin A, Armix 180-C®, and Linseed oil.

Another embodiment comprises cyclosporin A, Armix 183®, and Linseed oil.

Another embodiment comprises cyclosporin A, Armul 2404®, and Linseed oil.

Another embodiment comprises cyclosporin A, Armul 2404®, and Linseed oil.

Another embodiment comprises cyclosporin A, Atlas EMJ-C®, and Linseed oil.

Another embodiment comprises cyclosporin A, Atlas G-2109®, and Linseed oil.

Another embodiment comprises cyclosporin A, Atlas G-3886®, and Linseed oil.

Another embodiment comprises cyclosporin A, Atlas G-3890®, and Linseed oil.

Another embodiment comprises cyclosporin A, Bio Soft E-200®, and Linseed oil.

Another embodiment comprises cyclosporin A, Bio Soft E-300®, and Linseed oil.

Another embodiment comprises cyclosporin A, Bio Soft E-400®, and Linseed oil.

Another embodiment comprises cyclosporin A, Bio Soft EN 600®, and Linseed oil.

Another embodiment comprises cyclosporin A, Bio Soft TD-400®, and Linseed oil.

Another embodiment comprises cyclosporin A, Bio Soft TD-630®, and Linseed oil.

Another embodiment comprises cyclosporin A, Brij 30®, and Linseed oil.

Another embodiment comprises cyclosporin A, Brij 52®, and Linseed oil.

Another embodiment comprises cyclosporin A, Brij 56®, and Linseed oil.

Another embodiment comprises cyclosporin A, Brij 58®, and Linseed oil.

Another embodiment comprises cyclosporin A, Brij 72®, and Linseed oil.

Another embodiment comprises cyclosporin A, Brij 76®, and Linseed oil.

Another embodiment comprises cyclosporin A, Brij 78®, and Linseed oil.

Another embodiment comprises cyclosporin A, Brij 93®, and Linseed oil.

Another embodiment comprises cyclosporin A, Brij 97®, and Linseed oil.

Another embodiment comprises cyclosporin A, Brij 98®, and Linseed oil.

Another embodiment comprises cyclosporin A, Brij 700®, and Linseed oil.

Another embodiment comprises cyclosporin A, Brij 700 S®, and Linseed oil.

Another embodiment comprises cyclosporin A, Brij 721®, and Linseed oil.

Another embodiment comprises cyclosporin A, Brij 721 S®, and Linseed oil.

Another embodiment comprises cyclosporin A, Burcoterge CDG®, and Linseed oil.

Another embodiment comprises cyclosporin A, Canasol AT 600®, and Linseed oil.

Another embodiment comprises cyclosporin A, Canasol AT 800®, and Linseed oil.

Another embodiment comprises cyclosporin A, Canasol AT 1200®, and Linseed oil.

Another embodiment comprises cyclosporin A, Canasol BJ 35®, and Linseed oil.

Another embodiment comprises cyclosporin A, Canasol BJ 36®, and Linseed oil.

Another embodiment comprises cyclosporin A, Canasol BJ 52®, and Linseed oil.

Another embodiment comprises cyclosporin A, Canasol BJ 58®, and Linseed oil.

Another embodiment comprises cyclosporin A, Canasol BJ 72®, and Linseed oil.

Another embodiment comprises cyclosporin A, Canasol BJ 78®, and Linseed oil.

Another embodiment comprises cyclosporin A, Canasol BJ 98®, and Linseed oil.

Another embodiment comprises cyclosporin A, Canasol BJ 307®, and Linseed oil.

Another embodiment comprises cyclosporin A, Cerfak 1400®, and Linseed oil.

Another embodiment comprises cyclosporin A, Cetomacrogol 1000 BP®, and Linseed oil.

Another embodiment comprises cyclosporin A, Chemonic C-2®, and Linseed oil.

Another embodiment comprises cyclosporin A, Chemonic C-10®, and Linseed oil.

Another embodiment comprises cyclosporin A, Chemonic C-20®, and Linseed oil.

Another embodiment comprises cyclosporin A, Chemonic CT-12®, and Linseed oil.

Another embodiment comprises cyclosporin A, Chemonic CT-20®, and Linseed oil.

Another embodiment comprises cyclosporin A, Chemonic CT-30®, and Linseed oil.

Another embodiment comprises cyclosporin A, Chemonic CT-55®, and Linseed oil.

Another embodiment comprises cyclosporin A, Chemonic G-7®, and Linseed oil.

Another embodiment comprises cyclosporin A, Chemonic G-26®, and Linseed oil.

Another embodiment comprises cyclosporin A, Chemonic L-4A, and Linseed oil.

Another embodiment comprises cyclosporin A, Chemonic L-7®, and Linseed oil.

Another embodiment comprises cyclosporin A, Chemonic L-12®, and Linseed oil.

Another embodiment comprises cyclosporin A, Chemonic L-23®, and Linseed oil.

Another embodiment comprises cyclosporin A, Chemonic O-2®, and Linseed oil.

Another embodiment comprises cyclosporin A, Chemonic O-5®, and Linseed oil.

Another embodiment comprises cyclosporin A, Chemonic O-10®, and Linseed oil.

Another embodiment comprises cyclosporin A, Chemonic O-20®, and Linseed oil.

Another embodiment comprises cyclosporin A, Chemonic S-2®, and Linseed oil.

Another embodiment comprises cyclosporin A, Chemonic S-10®, and Linseed oil.

Another embodiment comprises cyclosporin A, Chemonic S-20®, and Linseed oil.

Another embodiment comprises cyclosporin A, Colamulse FE®, and Linseed oil.

Another embodiment comprises cyclosporin A, Cremophor A 20®, and Linseed oil.

Another embodiment comprises cyclosporin A, Cremophor SA 2®, and Linseed oil.

Another embodiment comprises cyclosporin A, Dehydol 100®, and Linseed oil.

Another embodiment comprises cyclosporin A, Dehydol O-4®, and Linseed oil.

Another embodiment comprises cyclosporin A, Delonic C-18®, and Linseed oil.

Another embodiment comprises cyclosporin A, DeSonic 6T®, and Linseed oil.

Another embodiment comprises cyclosporin A, DeSonic 9D®, and Linseed oil.

Another embodiment comprises cyclosporin A, DeSonic 9T®, and Linseed oil.

Another embodiment comprises cyclosporin A, DeSonic 12D®, and Linseed oil.

Another embodiment comprises cyclosporin A, DeSonic 12T®, and Linseed oil.

Another embodiment comprises cyclosporin A, DeSonic 15T®, and Linseed oil.

Another embodiment comprises cyclosporin A, DeSonic TDA-9®, and Linseed oil.

Another embodiment comprises cyclosporin A, DeThox GLG-7®, and Linseed oil.

Another embodiment comprises cyclosporin A, DeThox GLG-26®, and Linseed oil.

Another embodiment comprises cyclosporin A, DeThox LA-4®, and Linseed oil.

Another embodiment comprises cyclosporin A, DeThox LA-23®, and Linseed oil.

Another embodiment comprises cyclosporin A, DeThox SA-80®, and Linseed oil.

Another embodiment comprises cyclosporin A, Disponil O5®, and Linseed oil.

Another embodiment comprises cyclosporin A, Eccoterge EO-41 BO, and Linseed oil.

Another embodiment comprises cyclosporin A, Empilan KA2.5/90®, and Linseed oil.

Another embodiment comprises cyclosporin A, Empilan KA5/90®, and Linseed oil.

Another embodiment comprises cyclosporin A, Empilan KM-20®, and Linseed oil.

Another embodiment comprises cyclosporin A, Empilan KM-5®, and Linseed oil.

Another embodiment comprises cyclosporin A, Empilan L-23®, and Linseed oil.

Another embodiment comprises cyclosporin A, Ethylan 25-3®, and Linseed oil.

Another embodiment comprises cyclosporin A, Ethylan 1204®, and Linseed oil.

Another embodiment comprises cyclosporin A, Ethylan DA-4®, and Linseed oil.

Another embodiment comprises cyclosporin A, Ethylan LA-230®, and Linseed oil.

Another embodiment comprises cyclosporin A, Ethylan SN®, and Linseed oil.

Another embodiment comprises cyclosporin A, Ethylan TD-60®, and Linseed oil.

Another embodiment comprises cyclosporin A, Ethylan TD-100®, and Linseed oil.

Another embodiment comprises cyclosporin A, Ethylan TD-1407®, and Linseed oil.

Another embodiment comprises cyclosporin A, Eumulgin B1®, and Linseed oil.

Another embodiment comprises cyclosporin A, Eumulgin B2®, and Linseed oil.

Another embodiment comprises cyclosporin A, Eumulgin B3®, and Linseed oil.

Another embodiment comprises cyclosporin A, Eumulgin O-10®, and Linseed oil.

Another embodiment comprises cyclosporin A, Flo Mo 80/20®, and Linseed oil.

Another embodiment comprises cyclosporin A, Flo Mo Low Foam®, and Linseed oil.

Another embodiment comprises cyclosporin A, Forlan C-24®, and Linseed oil.

Another embodiment comprises cyclosporin A, Genapol 1454®, and Linseed oil.

Another embodiment comprises cyclosporin A, Genapol BA-020®, and Linseed oil.

Another embodiment comprises cyclosporin A, Genapol BA-040®, and Linseed oil.

Another embodiment comprises cyclosporin A, Genapol C-100®, and Linseed oil.

Another embodiment comprises cyclosporin A, Genapol DA 060®, and Linseed oil.

Another embodiment comprises cyclosporin A, Genapol HS 020®, and Linseed oil.

Another embodiment comprises cyclosporin A, Genapol HS 200®, and Linseed oil.

Another embodiment comprises cyclosporin A, Genapol ID-040®, and Linseed oil.

Another embodiment comprises cyclosporin A, Genapol ID-060®, and Linseed oil.

Another embodiment comprises cyclosporin A, Genapol ID-090®, and Linseed oil.

Another embodiment comprises cyclosporin A, Genapol LA 010®, and Linseed oil.

Another embodiment comprises cyclosporin A, Genapol LA 020®, and Linseed oil.

Another embodiment comprises cyclosporin A, Genapol LA 030®, and Linseed oil.

Another embodiment comprises cyclosporin A, Genapol LA 040®, and Linseed oil.

Another embodiment comprises cyclosporin A, Genapol LA 050®, and Linseed oil.

Another embodiment comprises cyclosporin A, Genapol LA 060®, and Linseed oil.

Another embodiment comprises cyclosporin A, Genapol LA 070®, and Linseed oil.

Another embodiment comprises cyclosporin A, Genapol LA 070S®, and Linseed oil.

Another embodiment comprises cyclosporin A, Genapol LA 230®, and Linseed oil.

Another embodiment comprises cyclosporin A, Genapol O 020®, and Linseed oil.

Another embodiment comprises cyclosporin A, Genapol O 050®, and Linseed oil.

Another embodiment comprises cyclosporin A, Genapol O 100®, and Linseed oil.

Another embodiment comprises cyclosporin A, Genapol O 200®, and Linseed oil.

Another embodiment comprises cyclosporin A, Genapol SA 030®, and Linseed oil.

Another embodiment comprises cyclosporin A, Genapbl SA 120®, and Linseed oil.

Another embodiment comprises cyclosporin A, Genapol T-020®, and Linseed oil.

Another embodiment comprises cyclosporin A, Genapol UD-030®, and Linseed oil.

Another embodiment comprises cyclosporin A, Genapol UD-050®, and Linseed oil.

Another embodiment comprises cyclosporin A, Genapol UD-070®, and Linseed oil.

Another embodiment comprises cyclosporin A, Genapol UD-079®, and Linseed oil.

Another embodiment comprises cyclosporin A, Genapol UD-080®, and Linseed oil.

Another embodiment comprises cyclosporin A, Genapol UD-110®, and Linseed oil.

Another embodiment comprises cyclosporin A, Genapol X 030®, and Linseed oil.

Another embodiment comprises cyclosporin A, Genapol X 050®, and Linseed oil.

Another embodiment comprises cyclosporin A, Genapol X 060®, and Linseed oil.

Another embodiment comprises cyclosporin A, Genapol X 070®, and Linseed oil.

Another embodiment comprises cyclosporin A, Genapol X 080®, and Linseed oil.

Another embodiment comprises cyclosporin A, Genapol X 100®, and Linseed oil.

Another embodiment comprises cyclosporin A, Genapol X 159®, and Linseed oil.

Another embodiment comprises cyclosporin A, Generol 122 E5®, and Linseed oil.

Another embodiment comprises cyclosporin A, Generol 122 E25®, and Linseed oil.

Another embodiment comprises cyclosporin A, Hostacerin T-3®, and Linseed oil.

Another embodiment comprises cyclosporin A, Iconol LF 110®, and Linseed oil.

Another embodiment comprises cyclosporin A, Incropol CS-20®, and Linseed oil.

Another embodiment comprises cyclosporin A, Lexemul CS-20®, and Linseed oil.

Another embodiment comprises cyclosporin A, Liponic EG-1®, and Linseed oil.

Another embodiment comprises cyclosporin A, Lipowax D®, and Linseed oil.

Another embodiment comprises cyclosporin A, Lipowax G®, and Linseed oil.

Another embodiment comprises cyclosporin A, Lipowax NI®, and Linseed oil.

Another embodiment comprises cyclosporin A, Lipowax P®, and Linseed oil.

Another embodiment comprises cyclosporin A, Lipowax P-31®, and Linseed oil.

Another embodiment comprises cyclosporin A, Lipowax PR®, and Linseed oil.

Another embodiment comprises cyclosporin A, Lumulse CS-20®, and Linseed oil.

Another embodiment comprises cyclosporin A, Macol CSA-20®, and Linseed oil.

Another embodiment comprises cyclosporin A, Marlox B 24/50®, and Linseed oil.

Another embodiment comprises cyclosporin A, Mazawet 77®, and Linseed oil.

Another embodiment comprises cyclosporin A, Norfox 1713®, and Linseed oil.

Another embodiment comprises cyclosporin A, Norfox 2579®, and Linseed oil.

Another embodiment comprises cyclosporin A, Norfox Lo Foam®, and Linseed oil.

Another embodiment comprises cyclosporin A, Promulgen D®, and Linseed oil.

Another embodiment comprises cyclosporin A, Promulgen G®, and Linseed oil.

Another embodiment comprises cyclosporin A, Renex 30®, and Linseed oil.

Another embodiment comprises cyclosporin A, Renex 36®, and Linseed oil.

Another embodiment comprises cyclosporin A, Rhodasurf A 24®, and Linseed oil.

Another embodiment comprises cyclosporin A, Rhodasurf AAE-10®, and Linseed oil.

Another embodiment comprises cyclosporin A, Rhodasurf BEH-25®, and Linseed oil.

Another embodiment comprises cyclosporin A, Rhodasurf BEH-40®, and Linseed oil.

Another embodiment comprises cyclosporin A, Rhodasurf DA 530®, and Linseed oil.

Another embodiment comprises cyclosporin A, Rhodasurf DA 630®, and Linseed oil.

Another embodiment comprises cyclosporin A, Rhodasurf DA 639®, and Linseed oil.

Another embodiment comprises cyclosporin A, Rhodasurf LAN-23®, and Linseed oil.

Another embodiment comprises cyclosporin A, Rhodasurf ON-870®, and Linseed oil.

Another embodiment comprises cyclosporin A, Rhodasurf ON-877®, and Linseed oil.

Another embodiment comprises cyclosporin A, Rhodasurf TB-970 FLK®, and Linseed oil.

Another embodiment comprises cyclosporin A, Ritacet-20®, and Linseed oil.

Another embodiment comprises cyclosporin A, Ritachol 1000®, and Linseed oil.

Another embodiment comprises cyclosporin A, Ritachol 2000®, and Linseed oil.

Another embodiment comprises cyclosporin A, Ritachol 5000®, and Linseed oil.

Another embodiment comprises cyclosporin A, Ritox 35®, and Linseed oil.

Another embodiment comprises cyclosporin A, Surfonic DA-4®, and Linseed oil.

Another embodiment comprises cyclosporin A, Surfonic DA-6®, and Linseed oil.

Another embodiment comprises cyclosporin A, Surfonic L46-7®, and Linseed oil.

Another embodiment comprises cyclosporin A, Sunonic POA®, and Linseed oil.

Another embodiment comprises cyclosporin A, Synthrapol KB®, and Linseed oil.

Another embodiment comprises cyclosporin A, Teginacid®, and Linseed oil.

Another embodiment comprises cyclosporin A, Teginacid C®, and Linseed oil.

Another embodiment comprises cyclosporin A, Tegotens EC 11®, and Linseed oil.

Another embodiment comprises cyclosporin A, Tinegal NA®, and Linseed oil.

Another embodiment comprises cyclosporin A, Tomadol 400®, and Linseed oil.

Another embodiment comprises cyclosporin A, Tomadol 600®, and Linseed oil.

Another embodiment comprises cyclosporin A, Tomadol 900®, and Linseed oil.

Another embodiment comprises cyclosporin A, Uniperol O®, and Linseed oil.

Another embodiment comprises cyclosporin A, Witconol SN Series®, and Linseed oil.

Another embodiment comprises cyclosporin A, an Ethoxylated Alkylphenol, and Linseed oil.

Another embodiment comprises cyclosporin A, Antarox LF-222®, and Linseed oil.

Another embodiment comprises cyclosporin A, Atlox 775®, and Linseed oil.

Another embodiment comprises cyclosporin A, Caloxylate N-9®, and Linseed oil.

Another embodiment comprises cyclosporin A, Canasol NF-1000®, and Linseed oil.

Another embodiment comprises cyclosporin A, Canasol NF-3000®, and Linseed oil.

Another embodiment comprises cyclosporin A, Canasol NF-3070®, and Linseed oil.

Another embodiment comprises cyclosporin A, Canasol OF 1670®, and Linseed oil.

Another embodiment comprises cyclosporin A, Canasol OF 2570®, and Linseed oil.

Another embodiment comprises cyclosporin A, Canasol OF 4070®, and Linseed oil.

Another embodiment comprises cyclosporin A, Chemax DNP-8®, and Linseed oil.

Another embodiment comprises cyclosporin A, Chemax DNP-18®, and Linseed oil.

Another embodiment comprises cyclosporin A, Chemax DNP-150/50®, and Linseed oil.

Another embodiment comprises cyclosporin A, DeSonic 1.5N®, and Linseed oil.

Another embodiment comprises cyclosporin A, DeSonic 4N®, and Linseed oil.

Another embodiment comprises cyclosporin A, DeSonic 5N®, and Linseed oil.

Another embodiment comprises cyclosporin A, DeSonic 6D®, and Linseed oil.

Another embodiment comprises cyclosporin A, DeSonic 6N®, and Linseed oil.

Another embodiment comprises cyclosporin A, DeSonic 7N®, and Linseed oil.

Another embodiment comprises cyclosporin A, DeSonic 9N®, and Linseed oil.

Another embodiment comprises cyclosporin A, DeSonic 10D®, and Linseed oil.

Another embodiment comprises cyclosporin A, DeSonic 11N®, and Linseed oil.

Another embodiment comprises cyclosporin A, DeSonic 12N®, and Linseed oil.

Another embodiment comprises cyclosporin A, DeSonic 15N®, and Linseed oil.

Another embodiment comprises cyclosporin A, DeSonic 20N®, and Linseed oil.

Another embodiment comprises cyclosporin A, Eccoscour RC®, and Linseed oil.

Another embodiment comprises cyclosporin A, Eccoterge EO-100®, and Linseed oil.

Another embodiment comprises cyclosporin A, Emulsifier 632/90%®, and Linseed oil.

Another embodiment comprises cyclosporin A, Geronol AG-821®, and Linseed oil.

Another embodiment comprises cyclosporin A, Gradonic N-95®, and Linseed oil.

Another embodiment comprises cyclosporin A, Hetoxide NP-4®, and Linseed oil.

Another embodiment comprises cyclosporin A, Hetoxide NP-30®, and Linseed oil.

Another embodiment comprises cyclosporin A, Hostapal N-100®, and Linseed oil.

Another embodiment comprises cyclosporin A, Hostapal N-110®, and Linseed oil.

Another embodiment comprises cyclosporin A, Igepal CTA-639W®, and Linseed oil.

Another embodiment comprises cyclosporin A, Igepal DAP-9®, and Linseed oil.

Another embodiment comprises cyclosporin A, Igepal OD-410®, and Linseed oil.

Another embodiment comprises cyclosporin A, Igepal SS-837®, and Linseed oil.

Another embodiment comprises cyclosporin A, Lipocol NP-9 USP®, and Linseed oil.

Another embodiment comprises cyclosporin A, Macol DNP-10®, and Linseed oil.

Another embodiment comprises cyclosporin A, Marlophen NP 5®, and Linseed oil.

Another embodiment comprises cyclosporin A, Marlophen P 1®, and Linseed oil.

Another embodiment comprises cyclosporin A, Surfonic NB®, and Linseed oil.

Another embodiment comprises cyclosporin A, Surfonic OPB-307®, and Linseed oil.

Another embodiment comprises cyclosporin A, Surfonic OPB-407®, and Linseed oil.

Another embodiment comprises cyclosporin A, Syn Fac 334®, and Linseed oil.

Another embodiment comprises cyclosporin A, Syn Fac 8216®, and Linseed oil.

Another embodiment comprises cyclosporin A, Triton N-57®, and Linseed oil.

Another embodiment comprises cyclosporin A, Trycol 6956®, and Linseed oil.

Another embodiment comprises cyclosporin A, Trycol 6961®, and Linseed oil.

Another embodiment comprises cyclosporin A, Trycol 6964®, and Linseed oil.

Another embodiment comprises cyclosporin A, Trycol 6969®, and Linseed oil.

Another embodiment comprises cyclosporin A, Trycol 6974®, and Linseed oil.

Another embodiment comprises cyclosporin A, Witbreak DRB-127®, and Linseed oil.

Another embodiment comprises cyclosporin A, Witbreak DRB-127®, and Linseed oil.

Another embodiment comprises cyclosporin A, Witconol NP Series®, and Linseed oil.

Another embodiment comprises cyclosporin A, an Ethoxylated Aryl Phenol, and Linseed oil.

Another embodiment comprises cyclosporin A, Soprophor BSU®, and Linseed oil.

Another embodiment comprises cyclosporin A, Soprophor CY/8®, and Linseed oil.

Another embodiment comprises cyclosporin A, Soprophor S/25®, and Linseed oil.

Another embodiment comprises cyclosporin A, Witconol NIO®, and Linseed oil.

Another embodiment comprises cyclosporin A, Witconol NIW®, and Linseed oil.

Another embodiment comprises cyclosporin A, Witconol S-100®, and Linseed oil.

Another embodiment comprises cyclosporin A, an Ethoxylated Fatty Acid, and Linseed oil.

Another embodiment comprises cyclosporin A, Aldo PGHMS®, and Linseed oil.

Another embodiment comprises cyclosporin A, Alkamuls TO-15/HR®, and Linseed oil.

Another embodiment comprises cyclosporin A, Armotan AL-69-66®, and Linseed oil.

Another embodiment comprises cyclosporin A, Cerasynt 840®, and Linseed oil.

Another embodiment comprises cyclosporin A, Cerasynt 945®, and Linseed oil.

Another embodiment comprises cyclosporin A, Crystal Inhibitor No. 5®, and Linseed oil.

Another embodiment comprises cyclosporin A, DeThox Acid L-9®, and Linseed oil.

Another embodiment comprises cyclosporin A, DeThox Acid S-8®, and Linseed oil.

Another embodiment comprises cyclosporin A, Ethofat 242/25®, and Linseed oil.

Another embodiment comprises cyclosporin A, Hydropalat 65®, and Linseed oil.

Another embodiment comprises cyclosporin A, Lipo EGMS®, and Linseed oil.

Another embodiment comprises cyclosporin A, Lipopeg 2 DL®, and Linseed oil.

Another embodiment comprises cyclosporin A, Lipopeg 4 DL®, and Linseed oil.

Another embodiment comprises cyclosporin A, Lipopeg 4-L®, and Linseed oil.

Another embodiment comprises cyclosporin A, Lipopeg 39-S®, and Linseed oil.

Another embodiment comprises cyclosporin A, Lipopeg 4-S®, and Linseed oil.

Another embodiment comprises cyclosporin A, Lipopeg 10-S®, and Linseed oil.

Another embodiment comprises cyclosporin A, Lipopeg 100-S®, and Linseed oil.

Another embodiment comprises cyclosporin A, Lipopeg 6000 DS®, and Linseed oil.

Another embodiment comprises cyclosporin A, Lumulse 40-L®, and Linseed oil.

Another embodiment comprises cyclosporin A, Lumulse 40-S®, and Linseed oil.

Another embodiment comprises cyclosporin A, Lumulse 42-L®, and Linseed oil.

Another embodiment comprises cyclosporin A, Lumulse 42-S®, and Linseed oil.

Another embodiment comprises cyclosporin A, Lumulse 100-S®, and Linseed oil.

Another embodiment comprises cyclosporin A, Lumulse 602-S®, and Linseed oil.

Another embodiment comprises cyclosporin A, Magrabar PGE-20-0®, and Linseed oil.

Another embodiment comprises cyclosporin A, Magrabar PGE-20L®, and Linseed oil.

Another embodiment comprises cyclosporin A, Magrabar PGE-20T®, and Linseed oil.

Another embodiment comprises cyclosporin A, Magrabar PGE-22-0®, and Linseed oil.

Another embodiment comprises cyclosporin A, Magrabar PGE-22L®, and Linseed oil.

Another embodiment comprises cyclosporin A, Magrabar PGE-22T®, and Linseed oil.

Another embodiment comprises cyclosporin A, Magrabar PGE-40-0®, and Linseed oil.

Another embodiment comprises cyclosporin A, Magrabar PGE-40L®, and Linseed oil.

Another embodiment comprises cyclosporin A, Magrabar PGE-40T®, and Linseed oil.

Another embodiment comprises cyclosporin A, Magrabar PGE-42-0®, and Linseed oil.

Another embodiment comprises cyclosporin A, Magrabar PGE-42L®, and Linseed oil.

Another embodiment comprises cyclosporin A, Magrabar PGE-42T®, and Linseed oil.

Another embodiment comprises cyclosporin A, Magrabar PGE-60-0®, and Linseed oil.

Another embodiment comprises cyclosporin A, Magrabar PGE-60L®, and Linseed oil.

Another embodiment comprises cyclosporin A, Magrabar PGE-60T®, and Linseed oil.

Another embodiment comprises cyclosporin A, Magrabar PGE-62-0®, and Linseed oil.

Another embodiment comprises cyclosporin A, Magrabar PGE-62L®, and Linseed oil.

Another embodiment comprises cyclosporin A, Magrabar PGE-62T®, and Linseed oil.

Another embodiment comprises cyclosporin A, Mapeg S-40K®, and Linseed oil.

Another embodiment comprises cyclosporin A, Marlowet OTS®, and Linseed oil.

Another embodiment comprises cyclosporin A, Naturechem PGR®, and Linseed oil.

Another embodiment comprises cyclosporin A, PG No. 4®, and Linseed oil.

Another embodiment comprises cyclosporin A, Renex 20®, and Linseed oil.

Another embodiment comprises cyclosporin A, Ritox 52®, and Linseed oil.

Another embodiment comprises cyclosporin A, Ritox 53®, and Linseed oil.

Another embodiment comprises cyclosporin A, Ritox 59®, and Linseed oil.

Another embodiment comprises cyclosporin A, Surfax 8916/A®, and Linseed oil.

Another embodiment comprises cyclosporin A, Tego Acid S 40 P®, and Linseed oil.

Another embodiment comprises cyclosporin A, Tego Acid S 100 P®, and Linseed oil.

Another embodiment comprises cyclosporin A, Tween 20®, and Linseed oil.

Another embodiment comprises cyclosporin A, Volpo 131®, and Linseed oil.

Another embodiment comprises cyclosporin A, an Ethoxylated Fatty Ester or Oil, and Linseed oil.

Another embodiment comprises cyclosporin A, Acconon 6-C10®, and Linseed oil.

Another embodiment comprises cyclosporin A, Acconon CC-6®, and Linseed oil.

Another embodiment comprises cyclosporin A, Acconon CO-7®, and Linseed oil.

Another embodiment comprises cyclosporin A, Aldosperse 40/60 FG®, and Linseed oil.

Another embodiment comprises cyclosporin A, Aldosperse ML-23®, and Linseed oil.

Another embodiment comprises cyclosporin A, Aldosperse MS-20 FG®, and Linseed oil.

Another embodiment comprises cyclosporin A, Alkamuls EL-620®, and Linseed oil.

Another embodiment comprises cyclosporin A, Alkamuls EL-719®, and Linseed oil.

Another embodiment comprises cyclosporin A, Alkamuls EL-985®, and Linseed oil.

Another embodiment comprises cyclosporin A, Arlatone G®, and Linseed oil.

Another embodiment comprises cyclosporin A, Arlatone T®, and Linseed oil.

Another embodiment comprises cyclosporin A, Atlas G-1045A®, and Linseed oil.

Another embodiment comprises cyclosporin A, Atlas G-1086®, and Linseed oil.

Another embodiment comprises cyclosporin A, Atlas G-1087®, and Linseed oil.

Another embodiment comprises cyclosporin A, Atlas G-1089®, and Linseed oil.

Another embodiment comprises cyclosporin A, Atlas G-1096®, and Linseed oil.

Another embodiment comprises cyclosporin A, Atlas G-1292®, and Linseed oil.

Another embodiment comprises cyclosporin A, Atlas G-1293®, and Linseed oil.

Another embodiment comprises cyclosporin A, Atlas G-1300®, and Linseed oil.

Another embodiment comprises cyclosporin A, Atlas G-7076®, and Linseed oil.

Another embodiment comprises cyclosporin A, Capmul EMG®, and Linseed oil.

Another embodiment comprises cyclosporin A, Chemonic CO-40®, and Linseed oil.

Another embodiment comprises cyclosporin A, Chemonic LI-3®, and Linseed oil.

Another embodiment comprises cyclosporin A, Chemonic LI-7®, and Linseed oil.

Another embodiment comprises cyclosporin A, Cirrasol GM®, and Linseed oil.

Another embodiment comprises cyclosporin A, Cremophor CO 40®, and Linseed oil.

Another embodiment comprises cyclosporin A, Cremophor CO 410®, and Linseed oil.

Another embodiment comprises cyclosporin A, Cremophor EL®, and Linseed oil.

Another embodiment comprises cyclosporin A, Cremophor GC7®, and Linseed oil.

Another embodiment comprises cyclosporin A, Cremophor RH-40®, and Linseed oil.

Another embodiment comprises cyclosporin A, Crovol A-40®, and Linseed oil.

Another embodiment comprises cyclosporin A, Crovol A-70®, and Linseed oil.

Another embodiment comprises cyclosporin A, Crovol M-70®, and Linseed oil.

Another embodiment comprises cyclosporin A, Crovol PK-70®, and Linseed oil.

Another embodiment comprises cyclosporin A, Cutina E-24®, and Linseed oil.

Another embodiment comprises cyclosporin A, Dacospin 12-R®, and Linseed oil.

Another embodiment comprises cyclosporin A, Dehymuls HRE-7®, and Linseed oil.

Another embodiment comprises cyclosporin A, DeSonic 30C®, and Linseed oil.

Another embodiment comprises cyclosporin A, DeSonic 36C®, and Linseed oil.

Another embodiment comprises cyclosporin A, DeSonic 40C®, and Linseed oil.

Another embodiment comprises cyclosporin A, Durfax 60®, and Linseed oil.

Another embodiment comprises cyclosporin A, Durfax 65®, and Linseed oil.

Another embodiment comprises cyclosporin A, Durfax 80®, and Linseed oil.

Another embodiment comprises cyclosporin A, Durfax EOM®, and Linseed oil.

Another embodiment comprises cyclosporin A, Eccoterge NF-2®, and Linseed oil.

Another embodiment comprises cyclosporin A, Emulpon CO-360®, and Linseed oil.

Another embodiment comprises cyclosporin A, Emulpon CO-550®, and Linseed oil.

Another embodiment comprises cyclosporin A, Emulsogen EL®, and Linseed oil.

Another embodiment comprises cyclosporin A, Emulsogen HCO 040®, and Linseed oil.

Another embodiment comprises cyclosporin A, Emulsogen HCO 060®, and Linseed oil.

Another embodiment comprises cyclosporin A, Emulsynt 1055®, and Linseed oil.

Another embodiment comprises cyclosporin A, Ethox 3095®, and Linseed oil.

Another embodiment comprises cyclosporin A, Eumulgin RO-40®, and Linseed oil.

Another embodiment comprises cyclosporin A, Genapol G-260®, and Linseed oil.

Another embodiment comprises cyclosporin A, Glycosperse L-20®, and Linseed oil.

Another embodiment comprises cyclosporin A, Glycosperse O-5®, and Linseed oil.

Another embodiment comprises cyclosporin A, Glycosperse O-20®, and Linseed oil.

Another embodiment comprises cyclosporin A, Glycosperse O-20 FG®, and Linseed oil.

Another embodiment comprises cyclosporin A, Glycosperse S-20®, and Linseed oil.

Another embodiment comprises cyclosporin A, Glycosperse S-20 FG®, and Linseed oil.

Another embodiment comprises cyclosporin A, Glycosperse TS-20®, and Linseed oil.

Another embodiment comprises cyclosporin A, Glycosperse TS-20 FG®, and Linseed oil.

Another embodiment comprises cyclosporin A, Hetan SL®, and Linseed oil.

Another embodiment comprises cyclosporin A, Hetan SO®, and Linseed oil.

Another embodiment comprises cyclosporin A, Hetan SS®, and Linseed oil.

Another embodiment comprises cyclosporin A, Hetoxide C-2®, and Linseed oil.

Another embodiment comprises cyclosporin A, Hetoxide C-9®, and Linseed oil.

Another embodiment comprises cyclosporin A, Hetoxide C-15®, and Linseed oil.

Another embodiment comprises cyclosporin A, Hetoxide C-25®, and Linseed oil.

Another embodiment comprises cyclosporin A, Hetoxide C-40®, and Linseed oil.

Another embodiment comprises cyclosporin A, Hetoxide C-200®, and Linseed oil.

Another embodiment comprises cyclosporin A, Hetoxide C-200-50%®, and Linseed oil.

Another embodiment comprises cyclosporin A, Hetoxide GC-30®, and Linseed oil.

Another embodiment comprises cyclosporin A, Hetoxide HC-60®, and Linseed oil.

Another embodiment comprises cyclosporin A, Ice No. 2®, and Linseed oil.

Another embodiment comprises cyclosporin A, Incrocas 30/40®, and Linseed oil.

Another embodiment comprises cyclosporin A, Lexol EC®, and Linseed oil.

Another embodiment comprises cyclosporin A, Lexol EO®, and Linseed oil.

Another embodiment comprises cyclosporin A, Lipocol HCO-40®, and Linseed oil.

Another embodiment comprises cyclosporin A, Lipocol HCO-60®, and Linseed oil.

Another embodiment comprises cyclosporin A, Lipocol O-3 Special®, and Linseed oil.

Another embodiment comprises cyclosporin A, Lipopeg 2-L®, and Linseed oil.

Another embodiment comprises cyclosporin A, Lipopeg 4-DO®, and Linseed oil.

Another embodiment comprises cyclosporin A, Lipopeg 4-DS®, and Linseed oil.

Another embodiment comprises cyclosporin A, Lipovol GTB®, and Linseed oil.

Another embodiment comprises cyclosporin A, Lonzest SML-20®, and Linseed oil.

Another embodiment comprises cyclosporin A, Lonzest SMO-20®, and Linseed oil.

Another embodiment comprises cyclosporin A, Lonzest SMS-20®, and Linseed oil.

Another embodiment comprises cyclosporin A, Lonzest STO-20®, and Linseed oil.

Another embodiment comprises cyclosporin A, Lonzest STS-20®, and Linseed oil.

Another embodiment comprises cyclosporin A, Lumulse GR-40®, and Linseed oil.

Another embodiment comprises cyclosporin A, Lumulse GRH-40®, and Linseed oil.

Another embodiment comprises cyclosporin A, Lumulse POE (7) GML®, and Linseed oil.

Another embodiment comprises cyclosporin A, Lumulse POE (12) Glyc®, and Linseed oil.

Another embodiment comprises cyclosporin A, Lumulse POE (40) MS KP®, and Linseed oil.

Another embodiment comprises cyclosporin A, Marlowet 4750®, and Linseed oil.

Another embodiment comprises cyclosporin A, Marlowet LVS®, and Linseed oil.

Another embodiment comprises cyclosporin A, Marlowet R 11®, and Linseed oil.

Another embodiment comprises cyclosporin A, Marlowet R 40®, and Linseed oil.

Another embodiment comprises cyclosporin A, Mazol 80 MGK®, and Linseed oil.

Another embodiment comprises cyclosporin A, Nonionic Emulsifier T-9®, and Linseed oil.

Another embodiment comprises cyclosporin A, Oronal LCG®, and Linseed oil.

Another embodiment comprises cyclosporin A, Polyderm PPI-CO-200®, and Linseed oil.

Another embodiment comprises cyclosporin A, Polyderm PPI-CO-40®, and Linseed oil.

Another embodiment comprises cyclosporin A, Rewoderm LI 520-70®, and Linseed oil.

Another embodiment comprises cyclosporin A, Ritapeg 150 DS®, and Linseed oil.

Another embodiment comprises cyclosporin A, Softigen 767®, and Linseed oil.

Another embodiment comprises cyclosporin A, Surfactol 318®, and Linseed oil.

Another embodiment comprises cyclosporin A, Surfactol 365®, and Linseed oil.

Another embodiment comprises cyclosporin A, Syn Lube 107®, and Linseed oil.

Another embodiment comprises cyclosporin A, Syn Lube 728®, and Linseed oil.

Another embodiment comprises cyclosporin A, Syn Lube 1632H®, and Linseed oil.

Another embodiment comprises cyclosporin A, Syn Lube 6277-A®, and Linseed oil.

Another embodiment comprises cyclosporin A, T-Det C-20®, and Linseed oil.

Another embodiment comprises cyclosporin A, T-Det C-40®, and Linseed oil.

Another embodiment comprises cyclosporin A, Tally 100 Plus®, and Linseed oil.

Another embodiment comprises cyclosporin A, Uniperol EL®, and Linseed oil.

Another embodiment comprises cyclosporin A, a Fatty Ester, and Linseed oil.

Another embodiment comprises cyclosporin A, Actralube-Syn 147®, and Linseed oil.

Another embodiment comprises cyclosporin A, Atlas G-1556®, and Linseed oil.

Another embodiment comprises cyclosporin A, Atlas G-1564®, and Linseed oil.

Another embodiment comprises cyclosporin A, Atlasol Base Oil S®, and Linseed oil.

Another embodiment comprises cyclosporin A, Base ML®, and Linseed oil.

Another embodiment comprises cyclosporin A, Base MT®, and Linseed oil.

Another embodiment comprises cyclosporin A, Cerasynt 303®, and Linseed oil.

Another embodiment comprises cyclosporin A, Dermol 1012®, and Linseed oil.

Another embodiment comprises cyclosporin A, Kemester 4000®, and Linseed oil.

Another embodiment comprises cyclosporin A, Lactipol S®, and Linseed oil.

Another embodiment comprises cyclosporin A, Magrabar PGO®, and Linseed oil.

Another embodiment comprises cyclosporin A, Mayco Base BFO®, and Linseed oil.

Another embodiment comprises cyclosporin A, Methyl Linoleate®, and Linseed oil.

Another embodiment comprises cyclosporin A, Pationic 122A®, and Linseed oil.

Another embodiment comprises cyclosporin A, Pationic 138C®, and Linseed oil.

Another embodiment comprises cyclosporin A, Pationic CSL®, and Linseed oil.

Another embodiment comprises cyclosporin A, Pationic ISL®, and Linseed oil.

Another embodiment comprises cyclosporin A, Pationic SBL®, and Linseed oil.

Another embodiment comprises cyclosporin A, Pationic SSL®, and Linseed oil.

Another embodiment comprises cyclosporin A, Ritasol®, and Linseed oil.

Another embodiment comprises cyclosporin A, Tego Alkanol CS 20®, and Linseed oil.

Another embodiment comprises cyclosporin A, Tego Alkanol L23 P®, and Linseed oil.

Another embodiment comprises cyclosporin A, Tego Alkanol S2®, and Linseed oil.

Another embodiment comprises cyclosporin A, Tego Alkanol S20 P®, and Linseed oil.

Another embodiment comprises cyclosporin A, Triemulsifier 600 MS®, and Linseed oil.

Another embodiment comprises cyclosporin A, a Glycerol Esters, and Linseed oil.

Another embodiment comprises cyclosporin A, Agro #9 Wint SBO®, and Linseed oil.

Another embodiment comprises cyclosporin A, Ahcovel Base 700®, and Linseed oil.

Another embodiment comprises cyclosporin A, Aldo HMS FG®, and Linseed oil.

Another embodiment comprises cyclosporin A, Aldo MLD®, and Linseed oil.

Another embodiment comprises cyclosporin A, Aldo MLD FG®, and Linseed oil.

Another embodiment comprises cyclosporin A, Aldo MO FG®, and Linseed oil.

Another embodiment comprises cyclosporin A, Aldo MS®, and Linseed oil.

Another embodiment comprises cyclosporin A, Aldo MS FG®, and Linseed oil.

Another embodiment comprises cyclosporin A, Aldo MS LG FG®, and Linseed oil.

Another embodiment comprises cyclosporin A, Aldo MSD®, and Linseed oil.

Another embodiment comprises cyclosporin A, Aldo MSD FG®, and Linseed oil.

Another embodiment comprises cyclosporin A, Aldosperse O-20 FG®, and Linseed oil.

Another embodiment comprises cyclosporin A, Aldosperse TS-20 FG®, and Linseed oil.

Another embodiment comprises cyclosporin A, Aldosperse TS-40 FG®, and Linseed oil.

Another embodiment comprises cyclosporin A, Arlacel 165®, and Linseed oil.

Another embodiment comprises cyclosporin A, Arlacel 186®, and Linseed oil.

Another embodiment comprises cyclosporin A, Capmul GMO®, and Linseed oil.

Another embodiment comprises cyclosporin A, Capmul GMS®, and Linseed oil.

Another embodiment comprises cyclosporin A, Caprol 3GO®, and Linseed oil.

Another embodiment comprises cyclosporin A, Caprol 3GVS®, and Linseed oil.

Another embodiment comprises cyclosporin A, Caprol 6G2S®, and Linseed oil.

Another embodiment comprises cyclosporin A, Caprol 10G40®, and Linseed oil.

Another embodiment comprises cyclosporin A, Caprol 10G100®, and Linseed oil.

Another embodiment comprises cyclosporin A, Caprol ET®, and Linseed oil.

Another embodiment comprises cyclosporin A, Caprol PGE860®, and Linseed oil.

Another embodiment comprises cyclosporin A, Cerasynt 945®, and Linseed oil.

Another embodiment comprises cyclosporin A, Cerasynt GMS®, and Linseed oil.

Another embodiment comprises cyclosporin A, Cerasynt Q®, and Linseed oil.

Another embodiment comprises cyclosporin A, Cerasynt SD®, and Linseed oil.

Another embodiment comprises cyclosporin A, Cerasynt WM®, and Linseed oil.

Another embodiment comprises cyclosporin A, Chemsperse 14®, and Linseed oil.

Another embodiment comprises cyclosporin A, Cremophor GO-32®, and Linseed oil.

Another embodiment comprises cyclosporin A, Cremophor GS11®, and Linseed oil.

Another embodiment comprises cyclosporin A, Cremophor GS-32®, and Linseed oil.

Another embodiment comprises cyclosporin A, Cutina KD-16®, and Linseed oil.

Another embodiment comprises cyclosporin A, Dehymuls PGPH®, and Linseed oil.

Another embodiment comprises cyclosporin A, Dermol DGDIS®, and Linseed oil.

Another embodiment comprises cyclosporin A, Dermol DGMIS®, and Linseed oil.

Another embodiment comprises cyclosporin A, Dermol G-76®, and Linseed oil.

Another embodiment comprises cyclosporin A, Dermol G-7DI®, and Linseed oil.

Another embodiment comprises cyclosporin A, Dermol NGDI®, and Linseed oil.

Another embodiment comprises cyclosporin A, Dermolan GLH®, and Linseed oil.

Another embodiment comprises cyclosporin A, Drewmulse GMO®, and Linseed oil.

Another embodiment comprises cyclosporin A, Drewpol 3-5-M®, and Linseed oil.

Another embodiment comprises cyclosporin A, Durlac 100 W®, and Linseed oil.

Another embodiment comprises cyclosporin A, Dur-Lo®, and Linseed oil.

Another embodiment comprises cyclosporin A, Dynasan 118®, and Linseed oil.

Another embodiment comprises cyclosporin A, EC-25®, and Linseed oil.

Another embodiment comprises cyclosporin A, EM 40®, and Linseed oil.

Another embodiment comprises cyclosporin A, Emerest 2400®, and Linseed oil.

Another embodiment comprises cyclosporin A, Emerest 2452®, and Linseed oil.

Another embodiment comprises cyclosporin A, Empilan G-26®, and Linseed oil.

Another embodiment comprises cyclosporin A, Genapol TSM®, and Linseed oil.

Another embodiment comprises cyclosporin A, Hostacerin DGI®, and Linseed oil.

Another embodiment comprises cyclosporin A, Hostacerin DGL®, and Linseed oil.

Another embodiment comprises cyclosporin A, Hostacerin DGMS®, and Linseed oil.

Another embodiment comprises cyclosporin A, Hostacerin DGSB®, and Linseed oil.

Another embodiment comprises cyclosporin A, Ice No. 2®, and Linseed oil.

Another embodiment comprises cyclosporin A, Imwitor 742®, and Linseed oil.

Another embodiment comprises cyclosporin A, Imwitor 780 K®, and Linseed oil.

Another embodiment comprises cyclosporin A, Imwitor 960 Flakes®, and Linseed oil.

Another embodiment comprises cyclosporin A, Isolan GI 34®, and Linseed oil.

Another embodiment comprises cyclosporin A, Isolan GO 33®, and Linseed oil.

Another embodiment comprises cyclosporin A, Kemester 1000®, and Linseed oil.

Another embodiment comprises cyclosporin A, Kemester 2000®, and Linseed oil.

Another embodiment comprises cyclosporin A, Kemester 2000®, and Linseed oil.

Another embodiment comprises cyclosporin A, Kemester 6000SE®, and Linseed oil.

Another embodiment comprises cyclosporin A, Lamecreme DGE 18®, and Linseed oil.

Another embodiment comprises cyclosporin A, Lexemul 515®, and Linseed oil.

Another embodiment comprises cyclosporin A, Lexemul 561®, and Linseed oil.

Another embodiment comprises cyclosporin A, Lexemul AR®, and Linseed oil.

Another embodiment comprises cyclosporin A, Lexemul AS®, and Linseed oil.

Another embodiment comprises cyclosporin A, Lexemul GDL®, and Linseed oil.

Another embodiment comprises cyclosporin A, Lexemul T®, and Linseed oil.

Another embodiment comprises cyclosporin A, Lipomulse 165®, and Linseed oil.

Another embodiment comprises cyclosporin A, Lumulse GML K®, and Linseed oil.

Another embodiment comprises cyclosporin A, Lumulse GMO K®, and Linseed oil.

Another embodiment comprises cyclosporin A, Lumulse GMR K®, and Linseed oil.

Another embodiment comprises cyclosporin A, Lumulse GMT K®, and Linseed oil.

Another embodiment comprises cyclosporin A, Magrabar GMC®, and Linseed oil.

Another embodiment comprises cyclosporin A, Magrabar GMO-CK®, and Linseed oil.

Another embodiment comprises cyclosporin A, Magrabar MDG-5050®, and Linseed oil.

Another embodiment comprises cyclosporin A, Magrabar PGO-315®, and Linseed oil.

Another embodiment comprises cyclosporin A, Magrabar PGO-1010®, and Linseed oil.

Another embodiment comprises cyclosporin A, Mazol 300K®, and Linseed oil.

Another embodiment comprises cyclosporin A, Mazol GMO-K®, and Linseed oil.

Another embodiment comprises cyclosporin A, Mazol GMS-K®, and Linseed oil.

Another embodiment comprises cyclosporin A, Mazol PG031-K®, and Linseed oil.

Another embodiment comprises cyclosporin A, Miglyol 812®, and Linseed oil.

Another embodiment comprises cyclosporin A, Norfox 165C®, and Linseed oil.

Another embodiment comprises cyclosporin A, Schercemol GMIS®, and Linseed oil.

Another embodiment comprises cyclosporin A, Tegin®, and Linseed oil.

Another embodiment comprises cyclosporin A, Tegin 4100 Pellets®, and Linseed oil.

Another embodiment comprises cyclosporin A, Tegin M Pellets®, and Linseed oil.

Another embodiment comprises cyclosporin A, Tegin OV®, and Linseed oil.

Another embodiment comprises cyclosporin A, Teginacid H®, and Linseed oil.

Another embodiment comprises cyclosporin A, Tego Cosmo P813®, and Linseed oil.

Another embodiment comprises cyclosporin A, Wickenol 535®, and Linseed oil.

Another embodiment comprises cyclosporin A, Witconol 14®, and Linseed oil.

Another embodiment comprises cyclosporin A, Witconol 14®, and Linseed oil.

Another embodiment comprises cyclosporin A, Witconol 14F®, and Linseed oil.

Another embodiment comprises cyclosporin A, Witconol 18L®, and Linseed oil.

Another embodiment comprises cyclosporin A, Witconol GOT®, and Linseed oil.

Another embodiment comprises cyclosporin A, Witconol MST®, and Linseed oil.

Another embodiment comprises cyclosporin A, Witconol RHT®, and Linseed oil.

Another embodiment comprises cyclosporin A, a Glycol Ester, and Linseed oil.

Another embodiment comprises cyclosporin A, Alkamuls 600 DO®, and Linseed oil.

Another embodiment comprises cyclosporin A, Alkamuls SEG®, and Linseed oil.

Another embodiment comprises cyclosporin A, Atlas EM-2®, and Linseed oil.

Another embodiment comprises cyclosporin A, Cerasynt IP®, and Linseed oil.

Another embodiment comprises cyclosporin A, Cerasynt M®, and Linseed oil.

Another embodiment comprises cyclosporin A, Cerasynt MN®, and Linseed oil.

Another embodiment comprises cyclosporin A, Cerasynt PA®, and Linseed oil.

Another embodiment comprises cyclosporin A, Chemsperse EGDS®, and Linseed oil.

Another embodiment comprises cyclosporin A, Chemsperse EGMS®, and Linseed oil.

Another embodiment comprises cyclosporin A, Colonial Monolaurin®, and Linseed oil.

Another embodiment comprises cyclosporin A, DeMuls SGE-95®, and Linseed oil.

Another embodiment comprises cyclosporin A, Eccoterge 200®, and Linseed oil.

Another embodiment comprises cyclosporin A, Emerest 2380®, and Linseed oil.

Another embodiment comprises cyclosporin A, Ethox 2610®, and Linseed oil.

Another embodiment comprises cyclosporin A, Ethox DO-9®, and Linseed oil.

Another embodiment comprises cyclosporin A, Ethox DO-14®, and Linseed oil.

Another embodiment comprises cyclosporin A, Ethox SO-9®, and Linseed oil.

Another embodiment comprises cyclosporin A, Fizul MD-318®, and Linseed oil.

Another embodiment comprises cyclosporin A, Genapol EGDS-VHP®, and Linseed oil.

Another embodiment comprises cyclosporin A, Genapol TS Powder®, and Linseed oil.

Another embodiment comprises cyclosporin A, Hostacerin WO®, and Linseed oil.

Another embodiment comprises cyclosporin A, Inversol 140®, and Linseed oil.

Another embodiment comprises cyclosporin A, Kemester 104®, and Linseed oil.

Another embodiment comprises cyclosporin A, Kemester 205®, and Linseed oil.

Another embodiment comprises cyclosporin A, Kemester 226®, and Linseed oil.

Another embodiment comprises cyclosporin A, Kemester 5221 SE®, and Linseed oil.

Another embodiment comprises cyclosporin A, Kemester EGDS®, and Linseed oil.

Another embodiment comprises cyclosporin A, Lexemul EGDS®, and Linseed oil.

Another embodiment comprises cyclosporin A, Lexemul EGMS®, and Linseed oil.

Another embodiment comprises cyclosporin A, Lexemul P®, and Linseed oil.

Another embodiment comprises cyclosporin A, Lipo DGLS®, and Linseed oil.

Another embodiment comprises cyclosporin A, Lipo EGDS®, and Linseed oil.

Another embodiment comprises cyclosporin A, Lipo PGMS®, and Linseed oil.

Another embodiment comprises cyclosporin A, Liposorb S-4®, and Linseed oil.

Another embodiment comprises cyclosporin A, Liposorb TO-20®, and Linseed oil.

Another embodiment comprises cyclosporin A, Lumulse PGO®, and Linseed oil.

Another embodiment comprises cyclosporin A, Mackester EGDS®, and Linseed oil.

Another embodiment comprises cyclosporin A, Mackester EGMS®, and Linseed oil.

Another embodiment comprises cyclosporin A, Mackester GSTP®, and Linseed oil.

Another embodiment comprises cyclosporin A, Mackester Series®, and Linseed oil.

Another embodiment comprises cyclosporin A, Magrabar PDG-50®, and Linseed oil.

Another embodiment comprises cyclosporin A, Mapeg 6000 DS®, and Linseed oil.

Another embodiment comprises cyclosporin A, Marlowet 4702®, and Linseed oil.

Another embodiment comprises cyclosporin A, Monalube 305®, and Linseed oil.

Another embodiment comprises cyclosporin A, Monalube 310®, and Linseed oil.

Another embodiment comprises cyclosporin A, Monalube 315®, and Linseed oil.

Another embodiment comprises cyclosporin A, Monalube 320®, and Linseed oil.

Another embodiment comprises cyclosporin A, Monalube 325®, and Linseed oil.

Another embodiment comprises cyclosporin A, Monalube 330®, and Linseed oil.

Another embodiment comprises cyclosporin A, Naturechem PGHS®, and Linseed oil.

Another embodiment comprises cyclosporin A, Polycastorol PLO-840®, and Linseed oil.

Another embodiment comprises cyclosporin A, Polytex 10M®, and Linseed oil.

Another embodiment comprises cyclosporin A, Ritasynt IP®, and Linseed oil.

Another embodiment comprises cyclosporin A, Ross Chem PEG 600 DT®, and Linseed oil.

Another embodiment comprises cyclosporin A, Schercemol PGMS®, and Linseed oil.

Another embodiment comprises cyclosporin A, Sponto H-44C®, and Linseed oil.

Another embodiment comprises cyclosporin A, Tegin G®, and Linseed oil.

Another embodiment comprises cyclosporin A, Witbreak DGE-182®, and Linseed oil.

Another embodiment comprises cyclosporin A, Witbreak DGE-182®, and Linseed oil.

Another embodiment comprises cyclosporin A, Witbreak DRA-21®, and Linseed oil.

Another embodiment comprises cyclosporin A, Witbreak DRA-21®, and Linseed oil.

Another embodiment comprises cyclosporin A, Witbreak DRA-50®, and Linseed oil.

Another embodiment comprises cyclosporin A, Witbreak DRA-50®, and Linseed oil.

Another embodiment comprises cyclosporin A, Witconol F26-46®, and Linseed oil.

Another embodiment comprises cyclosporin A, Witconol H-32®, and Linseed oil.

Another embodiment comprises cyclosporin A, Witconol H-33®, and Linseed oil.

Another embodiment comprises cyclosporin A, Witconol H-35A®, and Linseed oil.

Another embodiment comprises cyclosporin A, Witconol RHP®, and Linseed oil.

Another embodiment comprises cyclosporin A, a Lanolin-based Derivative, and Linseed oil.

Another embodiment comprises cyclosporin A, Amerchol CAB®, and Linseed oil.

Another embodiment comprises cyclosporin A, Amerchol L-101®, and Linseed oil.

Another embodiment comprises cyclosporin A, Amerlate LFA-LO®, and Linseed oil.

Another embodiment comprises cyclosporin A, Amerlate P®, and Linseed oil.

Another embodiment comprises cyclosporin A, Barre Common Degras®, and Linseed oil.

Another embodiment comprises cyclosporin A, Cholesterol NF®, and Linseed oil.

Another embodiment comprises cyclosporin A, Crodalan AWS®, and Linseed oil.

Another embodiment comprises cyclosporin A, Crodalan LA®, and Linseed oil.

Another embodiment comprises cyclosporin A, Emery 1650®, and Linseed oil.

Another embodiment comprises cyclosporin A, Emery 1650®, and Linseed oil.

Another embodiment comprises cyclosporin A, Emery 1740®, and Linseed oil.

Another embodiment comprises cyclosporin A, Emery 1740®, and Linseed oil.

Another embodiment comprises cyclosporin A, Forlan 500®, and Linseed oil.

Another embodiment comprises cyclosporin A, Forlan L®, and Linseed oil.

Another embodiment comprises cyclosporin A, Laneto 50®, and Linseed oil.

Another embodiment comprises cyclosporin A, Laneto 100®, and Linseed oil.

Another embodiment comprises cyclosporin A, Laneto AWS®, and Linseed oil.

Another embodiment comprises cyclosporin A, Lantrax 1776®, and Linseed oil.

Another embodiment comprises cyclosporin A, Lanfrax 1776®, and Linseed oil.

Another embodiment comprises cyclosporin A, Lanogel 21®, and Linseed oil.

Another embodiment comprises cyclosporin A, Lipolan®, and Linseed oil.

Another embodiment comprises cyclosporin A, Lipolan 31®, and Linseed oil.

Another embodiment comprises cyclosporin A, OHlan®, and Linseed oil.

Another embodiment comprises cyclosporin A, Polychol 5®, and Linseed oil.

Another embodiment comprises cyclosporin A, Polychol 15®, and Linseed oil.

Another embodiment comprises cyclosporin A, Ritacetyl®, and Linseed oil.

Another embodiment comprises cyclosporin A, Ritachol®, and Linseed oil.

Another embodiment comprises cyclosporin A, Ritahydrox®, and Linseed oil.

Another embodiment comprises cyclosporin A, Ritalafa®, and Linseed oil.

Another embodiment comprises cyclosporin A, Ritalan®, and Linseed oil.

Another embodiment comprises cyclosporin A, Ritalan AWS®, and Linseed oil.

Another embodiment comprises cyclosporin A, Ritalan C®, and Linseed oil.

Another embodiment comprises cyclosporin A, Ritawax®, and Linseed oil.

Another embodiment comprises cyclosporin A, Ritawax AEO®, and Linseed oil.

Another embodiment comprises cyclosporin A, Ritawax ALA®, and Linseed oil.

Another embodiment comprises cyclosporin A, Solan/Solan 50/Super Solan®, and Linseed oil.

Another embodiment comprises cyclosporin A, Super Hartolan/Hartolan®, and Linseed oil.

Another embodiment comprises cyclosporin A, Supersat AWS-4®, and Linseed oil.

Another embodiment comprises cyclosporin A, Supersat AWS-24®, and Linseed oil.

Another embodiment comprises cyclosporin A, a Lecithin or Lecithin Derivative, and Linseed oil.

Another embodiment comprises cyclosporin A, Alcolec®, and Linseed oil.

Another embodiment comprises cyclosporin A, Lecithin®, and Linseed oil.

Another embodiment comprises cyclosporin A, Lexin K®, and Linseed oil.

Another embodiment comprises cyclosporin A, Natipide®, and Linseed oil.

Another embodiment comprises cyclosporin A, a Lignin or Lignin Derivative, and Linseed oil.

Another embodiment comprises cyclosporin A, Diwatex XP 9®, and Linseed oil.

Another embodiment comprises cyclosporin A, Dynasperse LCD®, and Linseed oil.

Another embodiment comprises cyclosporin A, Indutin SAL®, and Linseed oil.

Another embodiment comprises cyclosporin A, Indulin W-1®, and Linseed oil.

Another embodiment comprises cyclosporin A, Indulin W-5®, and Linseed oil.

Another embodiment comprises cyclosporin A, Lignosot FTA®, and Linseed oil.

Another embodiment comprises cyclosporin A, Lignosol SFX-65®, and Linseed oil.

Another embodiment comprises cyclosporin A, Marasperse 52 CP®, and Linseed oil.

Another embodiment comprises cyclosporin A, Marasperse AG®, and Linseed oil.

Another embodiment comprises cyclosporin A, Marasperse CBOS-4®, and Linseed oil.

Another embodiment comprises cyclosporin A, Ufoxane 2®, and Linseed oil.

Another embodiment comprises cyclosporin A, a Methyl Ester, and Linseed oil.

Another embodiment comprises cyclosporin A, E.B. Cleaner AK®, and Linseed oil.

Another embodiment comprises cyclosporin A, Oleocal ME-70®, and Linseed oil.

Another embodiment comprises cyclosporin A, Trade Name Company®, and Linseed oil.

Another embodiment comprises cyclosporin A, Methyl Esters (cont'd)®, and Linseed oil.

Another embodiment comprises cyclosporin A, Oleocal ME-92®, and Linseed oil.

Another embodiment comprises cyclosporin A, Oleocal ME-112®, and Linseed oil.

Another embodiment comprises cyclosporin A, Oleocal ME-130®, and Linseed oil.

Another embodiment comprises cyclosporin A, a Monoglyceride or a Derivative thereof, and Linseed oil.

Another embodiment comprises cyclosporin A, Dynacet 211®, and Linseed oil.

Another embodiment comprises cyclosporin A, Hetsorb S-20®, and Linseed oil.

Another embodiment comprises cyclosporin A, Imwitor 191®, and Linseed oil.

Another embodiment comprises cyclosporin A, Imwitor 370®, and Linseed oil.

Another embodiment comprises cyclosporin A, Imwitor 375®, and Linseed oil.

Another embodiment comprises cyclosporin A, Imwitor 900®, and Linseed oil.

Another embodiment comprises cyclosporin A, Imwitor 945®, and Linseed oil.

Another embodiment comprises cyclosporin A, Imwitor 2020®, and Linseed oil.

Another embodiment comprises cyclosporin A, Kemester 5500®, and Linseed oil.

Another embodiment comprises cyclosporin A, Kemester 6000®, and Linseed oil.

Another embodiment comprises cyclosporin A, Magrabar GMC®, and Linseed oil.

Another embodiment comprises cyclosporin A, Magrabar GMO-CK®, and Linseed oil.

Another embodiment comprises cyclosporin A, Magrabar GPC-10®, and Linseed oil.

Another embodiment comprises cyclosporin A, Magrabar MDG-5050®, and Linseed oil.

Another embodiment comprises cyclosporin A, Monalube 335®, and Linseed oil.

Another embodiment comprises cyclosporin A, Monoglycerides Glyceryl Monestearate Archer Daniels Midland Company®, and Linseed oil.

Another embodiment comprises cyclosporin A, Rita GMS®, and Linseed oil.

Another embodiment comprises cyclosporin A, Ritamulse SCG®, and Linseed oil.

Another embodiment comprises cyclosporin A, Softigen 701®, and Linseed oil.

Another embodiment comprises cyclosporin A, Tally 100 Plus®, and Linseed oil.

Another embodiment comprises cyclosporin A, a Polyethylene Glycol, and Linseed oil.

Another embodiment comprises cyclosporin A, Emulgade PL 68/50®, and Linseed oil.

Another embodiment comprises cyclosporin A, Lumulse PEG®, and Linseed oil.

Another embodiment comprises cyclosporin A, Rhodasurf PEG-400®, and-Linseed oil.

Another embodiment comprises cyclosporin A, Rhodasurf PEG-600®, and Linseed oil.

Another embodiment comprises cyclosporin A, Witconol PEG-400®, and Linseed oil.

Another embodiment comprises cyclosporin A, a Polymeric Sudactant, and Linseed oil.

Another embodiment comprises cyclosporin A, Acritamer PNC-EG®, and Linseed oil.

Another embodiment comprises cyclosporin A, Ag-Rho DEP-775®, and Linseed oil.

Another embodiment comprises cyclosporin A, APG 325N Glycoside®, and Linseed oil.

Another embodiment comprises cyclosporin A, Aristoflex AVC®, and Linseed oil.

Another embodiment comprises cyclosporin A, Aristoflex HMB®, and Linseed oil.

Another embodiment comprises cyclosporin A, Burco NPS-225®, and Linseed oil.

Another embodiment comprises cyclosporin A, Burco NPS-816®, and Linseed oil.

Another embodiment comprises cyclosporin A, Chemccinate 5603®, and Linseed oil.

Another embodiment comprises cyclosporin A, Cosmedia Guar C-261N®, and Linseed oil.

Another embodiment comprises cyclosporin A, Gantrez S-95®, and Linseed oil.

Another embodiment comprises cyclosporin A, Glucopon 220 UP®, and Linseed oil.

Another embodiment comprises cyclosporin A, Glucopon 225 DK®, and Linseed oil.

Another embodiment comprises cyclosporin A, Glucopon 425 N®, and Linseed oil.

Another embodiment comprises cyclosporin A, Glucopon 600 UP®, and Linseed oil.

Another embodiment comprises cyclosporin A, Glucopon 625 UP®, and Linseed oil.

Another embodiment comprises cyclosporin A, Pemulen 1621®, and Linseed oil.

Another embodiment comprises cyclosporin A, Pemulen 1622®, and Linseed oil.

Another embodiment comprises cyclosporin A, Pemulen TR-1®, and Linseed oil.

Another embodiment comprises cyclosporin A, Pemulen TR-2®, and Linseed oil.

Another embodiment comprises cyclosporin A, Plantacare 818®, and Linseed oil.

Another embodiment comprises cyclosporin A, Plantapon LGC Sorb®, and Linseed oil.

Another embodiment comprises cyclosporin A, Plantaren 1200N®, and Linseed oil.

Another embodiment comprises cyclosporin A, Plantaren 2000N®, and Linseed oil.

Another embodiment comprises cyclosporin A, Viscolam AT 64®, and Linseed oil.

Another embodiment comprises cyclosporin A, Viscolam AT 64P®, and Linseed oil.

Another embodiment comprises cyclosporin A, Viscolam AT 100®, and Linseed oil.

Another embodiment comprises cyclosporin A, Viscolam MAC 7®, and Linseed oil.

Another embodiment comprises cyclosporin A, Viscolam SMC 20®, and Linseed oil.

Another embodiment comprises cyclosporin A, Witbreak RTC-323®, and Linseed oil.

Another embodiment comprises cyclosporin A, WSI 3700®, and Linseed oil.

Another embodiment comprises cyclosporin A, a Propoxylated or Ethoxylated Fatty Acid, Alcohol, or Alkyl Phenol, and Linseed oil.

Another embodiment comprises cyclosporin A, Antarox AA-60®, and Linseed oil.

Another embodiment comprises cyclosporin A, Antarox LF-224®, and Linseed oil.

Another embodiment comprises cyclosporin A, Burcomul DFE-45®, and Linseed oil.

Another embodiment comprises cyclosporin A, Burcoterge LFE-1000®, and Linseed oil.

Another embodiment comprises cyclosporin A, Chemal LF-25B®, and Linseed oil.

Another embodiment comprises cyclosporin A, Chemal LF-40B®, and Linseed oil.

Another embodiment comprises cyclosporin A, Dehypon LS-36®, and Linseed oil.

Another embodiment comprises cyclosporin A, Dehypon LS-36®, and Linseed oil.

Another embodiment comprises cyclosporin A, Dehypon LS-54®, and Linseed oil.

Another embodiment comprises cyclosporin A, Dehypon LS-54®, and Linseed oil.

Another embodiment comprises cyclosporin A, Delonic 100 VLF®, and Linseed oil.

Another embodiment comprises cyclosporin A, Delonic LF-60 MOD®, and Linseed oil.

Another embodiment comprises cyclosporin A, Empiderm B®, and Linseed oil.

Another embodiment comprises cyclosporin A, Ethylan 1206®, and Linseed oil.

Another embodiment comprises cyclosporin A, Ethylan NS-500K®, and Linseed oil.

Another embodiment comprises cyclosporin A, Ethylan NS-500LQ®, and Linseed oil.

Another embodiment comprises cyclosporin A, Genapol 1392®, and Linseed oil.

Another embodiment comprises cyclosporin A, Genapol 2317®, and Linseed oil.

Another embodiment comprises cyclosporin A, Genapol 26EP710®, and Linseed oil.

Another embodiment comprises cyclosporin A, Genapol EP 1022®, and Linseed oil.

Another embodiment comprises cyclosporin A, Genapol EP 1024®, and Linseed oil.

Another embodiment comprises cyclosporin A, Genapol EP 6068®, and Linseed oil.

Another embodiment comprises cyclosporin A, Genapol NP915®, and Linseed oil.

Another embodiment comprises cyclosporin A, Kieralon MFB®, and Linseed oil.

Another embodiment comprises cyclosporin A, Lumisolve CSA-80 V®, and Linseed oil.

Another embodiment comprises cyclosporin A, Marlowet 5001®, and Linseed oil.

Another embodiment comprises cyclosporin A, Marlox FK 64®, and Linseed oil.

Another embodiment comprises cyclosporin A, Marlox MO 124®, and Linseed oil.

Another embodiment comprises cyclosporin A, Marlox S 58®, and Linseed oil.

Another embodiment comprises cyclosporin A, Nonatell 1003®, and Linseed oil.

Another embodiment comprises cyclosporin A, Nonatell 1038®, and Linseed oil.

Another embodiment comprises cyclosporin A, Nonatell 1052®, and Linseed oil.

Another embodiment comprises cyclosporin A, Nonatell 1061®, and Linseed oil.

Another embodiment comprises cyclosporin A, Nonatell 1075®, and Linseed oil.

Another embodiment comprises cyclosporin A, Nonatell 1088®, and Linseed oil.

Another embodiment comprises cyclosporin A, Nonatell 1123®, and Linseed oil.

Another embodiment comprises cyclosporin A, Nonatell 1153®, and Linseed oil.

Another embodiment comprises cyclosporin A, Nonatell 1161®, and Linseed oil.

Another embodiment comprises cyclosporin A, Nonatell 1172®, and Linseed oil.

Another embodiment comprises cyclosporin A, Nonatell 1181®, and Linseed oil.

Another embodiment comprises cyclosporin A, Norfox 36®, and Linseed oil.

Another embodiment comprises cyclosporin A, Procetyl AWS®, and Linseed oil.

Another embodiment comprises cyclosporin A, Sandoxylate SX 412®, and Linseed oil.

Another embodiment comprises cyclosporin A, Sandoxylate SX 418®, and Linseed oil.

Another embodiment comprises cyclosporin A, Surfonic JL-80X®, and Linseed oil.

Another embodiment comprises cyclosporin A, Surfonic JL-80X-B1®, and Linseed oil.

Another embodiment comprises cyclosporin A, Surlonic L4-29X®, and Linseed oil.

Another embodiment comprises cyclosporin A, Surfonic LF®, and Linseed oil.

Another embodiment comprises cyclosporin A, T-Det A826®, and Linseed oil.

Another embodiment comprises cyclosporin A, T-Det LF-416®, and Linseed oil.

Another embodiment comprises cyclosporin A, Tergitol Min-Foam 1X®, and Linseed oil.

Another embodiment comprises cyclosporin A, Tergitol Min-Foam 2X®, and Linseed oil.

Another embodiment comprises cyclosporin A, Triton CF-21®, and Linseed oil.

Another embodiment comprises cyclosporin A, Triton CF-76®, and Linseed oil.

Another embodiment comprises cyclosporin A, Triton XL-80N®, and Linseed oil.

Another embodiment comprises cyclosporin A, Witconol NS-98®, and Linseed oil.

Another embodiment comprises cyclosporin A, Witconol NS-108LQ®, and Linseed oil.

Another embodiment comprises cyclosporin A, Witconol NS-145®, and Linseed oil.

Another embodiment comprises cyclosporin A, Witconol NS-179®, and Linseed oil.

Another embodiment comprises cyclosporin A, a Protein-based Surfactant, and Linseed oil.

Another embodiment comprises cyclosporin A, AminoFoam W®, and Linseed oil.

Another embodiment comprises cyclosporin A, Amiter LGOD-2®, and Linseed oil.

Another embodiment comprises cyclosporin A, Amiter LGS-2®, and Linseed oil.

Another embodiment comprises cyclosporin A, Amiter LGS-5®, and Linseed oil.

Another embodiment comprises cyclosporin A, Lamepon S®, and Linseed oil.

Another embodiment comprises cyclosporin A, Lamepon S®, and Linseed oil.

Another embodiment comprises cyclosporin A, Maypon 4C®, and Linseed oil.

Another embodiment comprises cyclosporin A, May-Tein C®, and Linseed oil.

Another embodiment comprises cyclosporin A, May-Tein CT®, and Linseed oil.

Another embodiment comprises cyclosporin A, May-Tein KTS®, and Linseed oil.

Another embodiment comprises cyclosporin A, May-Tein SY®, and Linseed oil.

Another embodiment comprises cyclosporin A, Plantapon S®, and Linseed oil.

Another embodiment comprises cyclosporin A, Proteol APL®, and Linseed oil.

Another embodiment comprises cyclosporin A, Proteol OAT®, and Linseed oil.

Another embodiment comprises cyclosporin A, Pyroter CPI-40®, and Linseed oil.

Another embodiment comprises cyclosporin A, Pyroter GPI-25®, and Linseed oil.

Another embodiment comprises cyclosporin A, Supro-Tein S®, and Linseed oil.

Another embodiment comprises cyclosporin A, Supro-Tein V®, and Linseed oil.

Another embodiment comprises cyclosporin A,® and Linseed oil.

Another embodiment comprises cyclosporin A, a Sarcosine Derivative®, and Linseed oil.

Another embodiment comprises cyclosporin A, Crodasinic LS-30®, and Linseed oil.

Another embodiment comprises cyclosporin A, Vanseal CS®, and Linseed oil.

Another embodiment comprises cyclosporin A, Vanseal LS®, and Linseed oil.

Another embodiment comprises cyclosporin A, Vanseal MS®, and Linseed oil.

Another embodiment comprises cyclosporin A, Vanseal NACS-30®, and Linseed oil.

Another embodiment comprises cyclosporin A, Vanseal NALS-95®, and Linseed oil.

Another embodiment comprises cyclosporin A, Vanseal OS®, and Linseed oil.

Another embodiment comprises cyclosporin A,® and Linseed oil.

Another embodiment comprises cyclosporin A, a Silicone-based Surfactant®, and Linseed oil.

Another embodiment comprises cyclosporin A, Abil-B-9950®, and Linseed oil.

Another embodiment comprises cyclosporin A, Abil Care 85®, and Linseed oil.

Another embodiment comprises cyclosporin A, Abil EM 90®, and Linseed oil.

Another embodiment comprises cyclosporin A, Abil EM 97®, and Linseed oil.

Another embodiment comprises cyclosporin A, Abil WE-09®, and Linseed oil.

Another embodiment comprises cyclosporin A, Dow Corning 1248 Fluid®, and Linseed oil.

Another embodiment comprises cyclosporin A, Dow Corning 3225C®, and Linseed oil.

Another embodiment comprises cyclosporin A, Dow Corning 5200®, and Linseed oil.

Another embodiment comprises cyclosporin A, Dow Corning Q4-3667®, and Linseed oil.

Another embodiment comprises cyclosporin A, Monasil PCA®, and Linseed oil.

Another embodiment comprises cyclosporin A, Monasil PDM®, and Linseed oil.

Another embodiment comprises cyclosporin A, Monasil PLN®, and Linseed oil.

Another embodiment comprises cyclosporin A, Polyderm PPI-SI-WS®, and Linseed oil.

Another embodiment comprises cyclosporin A, Troysol 380W®, and Linseed oil.

Another embodiment comprises cyclosporin A, Troysol S366®, and Linseed oil.

Another embodiment comprises cyclosporin A, a Sorbitan Derivative, and Linseed oil.

Another embodiment comprises cyclosporin A, Alkamuls SML®, and Linseed oil.

Another embodiment comprises cyclosporin A, Alkamuls SMO®, and Linseed oil.

Another embodiment comprises cyclosporin A, Alkamuls STO®, and Linseed oil.

Another embodiment comprises cyclosporin A, Arlacel 20®, and Linseed oil.

Another embodiment comprises cyclosporin A, Arlacel 40®, and Linseed oil.

Another embodiment comprises cyclosporin A, Arlacel 60®, and Linseed oil.

Another embodiment comprises cyclosporin A, Arlacel 80®, and Linseed oil.

Another embodiment comprises cyclosporin A, Arlacel C®, and Linseed oil.

Another embodiment comprises cyclosporin A, Armul 21®, and Linseed oil.

Another embodiment comprises cyclosporin A, Atlox 80®, and Linseed oil.

Another embodiment comprises cyclosporin A, Atlox 847®, and Linseed oil.

Another embodiment comprises cyclosporin A, Atlox 1045A®, and Linseed oil.

Another embodiment comprises cyclosporin A, Canarcel 20®, and Linseed oil.

Another embodiment comprises cyclosporin A, Canarcel 603®, and Linseed oil.

Another embodiment comprises cyclosporin A, Canarcel 80®, and Linseed oil.

Another embodiment comprises cyclosporin A, Canarcel TW 20®, and Linseed oil.

Another embodiment comprises cyclosporin A, Canarcel TW 60®, and Linseed oil.

Another embodiment comprises cyclosporin A, Canarcel TW 80®, and Linseed oil.

Another embodiment comprises cyclosporin A, Coladet BSB-P®, and Linseed oil.

Another embodiment comprises cyclosporin A, Customulse O-20®, and Linseed oil.

Another embodiment comprises cyclosporin A, Dehymuls E®, and Linseed oil.

Another embodiment comprises cyclosporin A, DeSotan SMO®, and Linseed oil.

Another embodiment comprises cyclosporin A, DeSotan SMO-20®, and Linseed oil.

Another embodiment comprises cyclosporin A, DeSotan SMT®, and Linseed oil.

Another embodiment comprises cyclosporin A, DeSotan SMT-20®, and Linseed oil.

Another embodiment comprises cyclosporin A, Durfax 60®, and Linseed oil.

Another embodiment comprises cyclosporin A, Durfax 65®, and Linseed oil.

Another embodiment comprises cyclosporin A, Durfax 80®, and Linseed oil.

Another embodiment comprises cyclosporin A, Durtan 60®, and Linseed oil.

Another embodiment comprises cyclosporin A, Durtan 65®, and Linseed oil.

Another embodiment comprises cyclosporin A, Liposorb L®, and Linseed oil.

Another embodiment comprises cyclosporin A, Liposorb L-10®, and Linseed oil.

Another embodiment comprises cyclosporin A, Liposorb L-20®, and Linseed oil.

Another embodiment comprises cyclosporin A, Liposorb O®, and Linseed oil.

Another embodiment comprises cyclosporin A, Liposorb O-20®, and Linseed oil.

Another embodiment comprises cyclosporin A, Liposorb P®, and Linseed oil.

Another embodiment comprises cyclosporin A, Liposorb P-20®, and Linseed oil.

Another embodiment comprises cyclosporin A, Liposorb S®, and Linseed oil.

Another embodiment comprises cyclosporin A, Liposorb S-20®, and Linseed oil.

Another embodiment comprises cyclosporin A, Liposorb SQO®, and Linseed oil.

Another embodiment comprises cyclosporin A, Liposorb TO®, and Linseed oil.

Another embodiment comprises cyclosporin A, Liposorb TS®, and Linseed oil.

Another embodiment comprises cyclosporin A, Liposorb TS-20®, and Linseed oil.

Another embodiment comprises cyclosporin A, Lumisorb PS®, and Linseed oil.

Another embodiment comprises cyclosporin A, Lumisorb SMO (T)®, and Linseed oil.

Another embodiment comprises cyclosporin A, Lumisorb SMS K®, and Linseed oil.

Another embodiment comprises cyclosporin A, Lumisorb SSO®, and Linseed oil.

Another embodiment comprises cyclosporin A, Lumisorb STS K®, and Linseed oil.

Another embodiment comprises cyclosporin A, Lumisorb STT®, and Linseed oil.

Another embodiment comprises cyclosporin A, Magrabar SMO®, and Linseed oil.

Another embodiment comprises cyclosporin A, Magrabar SMO-VEG®, and Linseed oil.

Another embodiment comprises cyclosporin A, Magrabar SMT®, and Linseed oil.

Another embodiment comprises cyclosporin A, Magrabar STO®, and Linseed oil.

Another embodiment comprises cyclosporin A, Miracare BC-27®, and Linseed oil.

Another embodiment comprises cyclosporin A, Ritabate 20®, and Linseed oil.

Another embodiment comprises cyclosporin A, Ritabate 40®, and Linseed oil.

Another embodiment comprises cyclosporin A, Ritabate 60®, and Linseed oil.

Another embodiment comprises cyclosporin A, Ritabate 80®, and Linseed oil.

Another embodiment comprises cyclosporin A, T-Maz®, and Linseed oil.

Another embodiment comprises cyclosporin A, Tego SML®, and Linseed oil.

Another embodiment comprises cyclosporin A, Tego SML 20®, and Linseed oil.

Another embodiment comprises cyclosporin A, Tego SMO 80 V®, and Linseed oil.

Another embodiment comprises cyclosporin A, Tego SMO V®, and Linseed oil.

Another embodiment comprises cyclosporin A, Tego SMS®, and Linseed oil.

Another embodiment comprises cyclosporin A, Tego STO V®, and Linseed oil.

Another embodiment comprises cyclosporin A, Tween 21®, and Linseed oil.

Another embodiment comprises cyclosporin A, Tween 40®, and Linseed oil.

Another embodiment comprises cyclosporin A, Tween 60®, and Linseed oil.

Another embodiment comprises cyclosporin A, Tween 60 K®, and Linseed oil.

Another embodiment comprises cyclosporin A, Tween 61®, and Linseed oil.

Another embodiment comprises cyclosporin A, Tween 65®, and Linseed oil.

Another embodiment comprises cyclosporin A, Tween 8®, and Linseed oil.

Another embodiment comprises cyclosporin A, Tween 80 K®, and Linseed oil.

Another embodiment comprises cyclosporin A, Tween 81®, and Linseed oil.

Another embodiment comprises cyclosporin A, Tween 85®, and Linseed oil.

Another embodiment comprises cyclosporin A, a Sucrose or Glucose Ester, or Derivative thereof, and Linseed oil.

Another embodiment comprises cyclosporin A, DeSult GOS-P-60WCG®, and Linseed oil.

Another embodiment comprises cyclosporin A, Glucam E-20 Distearate®, and Linseed oil.

Another embodiment comprises cyclosporin A, Glucamate DOE-120®, and Linseed oil.

Another embodiment comprises cyclosporin A, Glucamate SSE-20®, and Linseed oil.

Another embodiment comprises cyclosporin A, Glucate DO®, and Linseed oil.

Another embodiment comprises cyclosporin A, Glucate SS®, and Linseed oil.

Another embodiment comprises cyclosporin A, Glucopon 425 UP®, and Linseed oil.

Another embodiment comprises cyclosporin A, Isolan IS®, and Linseed oil.

Another embodiment comprises cyclosporin A, Mazon 40®, and Linseed oil.

Another embodiment comprises cyclosporin A, Montanov 82®, and Linseed oil.

Another embodiment comprises cyclosporin A, Montanov 202®, and Linseed oil.

Another embodiment comprises cyclosporin A, Montanov S®, and Linseed oil.

Another embodiment comprises cyclosporin A, Rheozan®, and Linseed oil.

Another embodiment comprises cyclosporin A, Simulsol AS 48®, and Linseed oil.

Another embodiment comprises cyclosporin A, Simulsol SL 4®, and Linseed oil.

Another embodiment comprises cyclosporin A, Simulsol SL 10®, and Linseed oil.

Another embodiment comprises cyclosporin A, Simulsol SL 11W®, and Linseed oil.

Another embodiment comprises cyclosporin A, Simulsol SL 55®, and Linseed oil.

Another embodiment comprises cyclosporin A, Suga Nate 100 and 160®, and Linseed oil.

Another embodiment comprises cyclosporin A, Tego Care 450®, and Linseed oil.

Another embodiment comprises cyclosporin A, Tego Care CG 90®, and Linseed oil.

Another embodiment comprises cyclosporin A, Tego Care PS®, and Linseed oil.

Another embodiment comprises cyclosporin A, Tegosoft PSE 141 G®, and Linseed oil.

Another embodiment comprises cyclosporin A, Tegotens G 826®, and Linseed oil.

Another embodiment comprises cyclosporin A, Triton BG-10 (70%)®, and Linseed oil.

Another embodiment comprises cyclosporin A, Triton CG-110 (60%)®, and Linseed oil.

Another embodiment comprises cyclosporin A, Wickenol 545®, and Linseed oil.

Another embodiment comprises cyclosporin A, an alcohol, and Rapeseed oil.

Another embodiment comprises cyclosporin A, Diglycerol®, and Rapeseed oil.

Another embodiment comprises cyclosporin A, Hetoxide GT-80®, and Rapeseed oil.

Another embodiment comprises cyclosporin A, Lexemul BEO®, and Rapeseed oil.

Another embodiment comprises cyclosporin A, Polyglycerol-3®, and Rapeseed oil.

Another embodiment comprises cyclosporin A, Redicote E Series®, and Rapeseed oil.

Another embodiment comprises cyclosporin A, Simulsol OX 1005L®, and Rapeseed oil.

Another embodiment comprises cyclosporin A, Stanfax 567®, and Rapeseed oil.

Another embodiment comprises cyclosporin A, TA-1618®, and Rapeseed oil.

Another embodiment comprises cyclosporin A, Witconol H-31A®, and Rapeseed oil.

Another embodiment comprises cyclosporin A, an amine oxide, and Rapeseed oil.

Another embodiment comprises cyclosporin A, AO-405®, and Rapeseed oil.

Another embodiment comprises cyclosporin A, AO-455®, and Rapeseed oil.

Another embodiment comprises cyclosporin A, AO 728 Special®, and Rapeseed oil.

Another embodiment comprises cyclosporin A, Barlox 12®, and Rapeseed oil.

Another embodiment comprises cyclosporin A, Barlox 14®, and Rapeseed oil.

Another embodiment comprises cyclosporin A, Burcoxide Lo®, and Rapeseed oil.

Another embodiment comprises cyclosporin A, Caloxamine LO®, and Rapeseed oil.

Another embodiment comprises cyclosporin A, Chemoxide CAW®, and Rapeseed oil.

Another embodiment comprises cyclosporin A, Chemoxide LM-30®, and Rapeseed oil.

Another embodiment comprises cyclosporin A, Chemoxide LO®, and Rapeseed oil.

Another embodiment comprises cyclosporin A, Chemoxide MO®, and Rapeseed oil.

Another embodiment comprises cyclosporin A, Colalux CAO-35®, and Rapeseed oil.

Another embodiment comprises cyclosporin A, Colalux LO®, and Rapeseed oil.

Another embodiment comprises cyclosporin A, DeMox CAPO®, and Rapeseed oil.

Another embodiment comprises cyclosporin A, DeMox CSG-30®, and Rapeseed oil.

Another embodiment comprises cyclosporin A, DeMox LAO®, and Rapeseed oil.

Another embodiment comprises cyclosporin A, Emcol L®, and Rapeseed oil.

Another embodiment comprises cyclosporin A, Empigen OB®, and Rapeseed oil.

Another embodiment comprises cyclosporin A, Empigen OS/A®, and Rapeseed oil.

Another embodiment comprises cyclosporin A, Foamox CDO®, and Rapeseed oil.

Another embodiment comprises cyclosporin A, Foamox DMM®, and Rapeseed oil.

Another embodiment comprises cyclosporin A, Foamox DMS®, and Rapeseed oil.

Another embodiment comprises cyclosporin A, Genaminox KC®, and Rapeseed oil.

Another embodiment comprises cyclosporin A, Genaminox LA®, and Rapeseed oil.

Another embodiment comprises cyclosporin A, Hartofoam SAO®, and Rapeseed oil.

Another embodiment comprises cyclosporin A, Hartox DMCD®, and Rapeseed oil.

Another embodiment comprises cyclosporin A, Lipowax DAT®, and Rapeseed oil.

Another embodiment comprises cyclosporin A, Lipowax PB Pastilles®, and Rapeseed oil.

Another embodiment comprises cyclosporin A, Mackamine C8®, and Rapeseed oil.

Another embodiment comprises cyclosporin A, Mackamine C10®, and Rapeseed oil.

Another embodiment comprises cyclosporin A, Mackamine C14®, and Rapeseed oil.

Another embodiment comprises cyclosporin A, Mackamine CAO®, and Rapeseed oil.

Another embodiment comprises cyclosporin A, Mackamine CO®, and Rapeseed oil.

Another embodiment comprises cyclosporin A, Mackamine LO®, and Rapeseed oil.

Another embodiment comprises cyclosporin A, Mackamine O2®, and Rapeseed oil.

Another embodiment comprises cyclosporin A, Mackamine SAO®, and Rapeseed oil.

Another embodiment comprises cyclosporin A, Mackamine SO®, and Rapeseed oil.

Another embodiment comprises cyclosporin A, Mazox KCAO®, and Rapeseed oil.

Another embodiment comprises cyclosporin A, Monalac MO®, and Rapeseed oil.

Another embodiment comprises cyclosporin A, Norfox LDA®, and Rapeseed oil.

Another embodiment comprises cyclosporin A, Rhodamox LO®, and Rapeseed oil.

Another embodiment comprises cyclosporin A, Schercamox C-AA®, and Rapeseed oil.

Another embodiment comprises cyclosporin A, Schercamox DMA®, and Rapeseed oil.

Another embodiment comprises cyclosporin A, Schercamox DML®, and Rapeseed oil.

Another embodiment comprises cyclosporin A, Schercamox DMM®, and Rapeseed oil.

Another embodiment comprises cyclosporin A, Schercamox DMS®, and Rapeseed oil.

Another embodiment comprises cyclosporin A, Tegotens DO®, and Rapeseed oil.

Another embodiment comprises cyclosporin A, Tomah AO-14-2®, and Rapeseed oil.

Another embodiment comprises cyclosporin A, Triaminox CDO®, and Rapeseed oil.

Another embodiment comprises cyclosporin A, a block polymer, and Rapeseed oil.

Another embodiment comprises cyclosporin A, AL 2070®, and Rapeseed oil.

Another embodiment comprises cyclosporin A, Antarox 17-R-2®, and Rapeseed oil.

Another embodiment comprises cyclosporin A, Antarox 25-R-2®, and Rapeseed oil.

Another embodiment comprises cyclosporin A, Antarox 31-R-1®, and Rapeseed oil.

Another embodiment comprises cyclosporin A, Antarox P-84®, and Rapeseed oil.

Another embodiment comprises cyclosporin A, Antarox P-104/H®, and Rapeseed oil.

Another embodiment comprises cyclosporin A, Arnox BP-Series®, and Rapeseed oil.

Another embodiment comprises cyclosporin A, Chemonic 435®, and Rapeseed oil.

Another embodiment comprises cyclosporin A, Chemonic D-25®, and Rapeseed oil.

Another embodiment comprises cyclosporin A, Chemonic PL Series®, and Rapeseed oil.

Another embodiment comprises cyclosporin A, Ethox L-121®, and Rapeseed oil.

Another embodiment comprises cyclosporin A, Ethox L-122®, and Rapeseed oil.

Another embodiment comprises cyclosporin A, Genapol PF-10®, and Rapeseed oil.

Another embodiment comprises cyclosporin A, Genapol PF-20®, and Rapeseed oil.

Another embodiment comprises cyclosporin A, Genapol PF-40A®, and Rapeseed oil.

Another embodiment comprises cyclosporin A, Norfox 2-LF®, and Rapeseed oil.

Another embodiment comprises cyclosporin A, Simulsol NW 342®, and Rapeseed oil.

Another embodiment comprises cyclosporin A, T-Det BP-1®, and Rapeseed oil.

Another embodiment comprises cyclosporin A, T-Det XD®, and Rapeseed oil.

Another embodiment comprises cyclosporin A, T-Det XH®, and Rapeseed oil.

Another embodiment comprises cyclosporin A, Triton CF-32®, and Rapeseed oil.

Another embodiment comprises cyclosporin A, Witconol 171®, and Rapeseed oil.

Another embodiment comprises cyclosporin A, Witconol 324®, and Rapeseed oil.

Another embodiment comprises cyclosporin A, Witconol 324D®, and Rapeseed oil.

Another embodiment comprises cyclosporin A, Witconol PD-2000®, and Rapeseed oil.

Another embodiment comprises cyclosporin A, a Carboxylated Alcohol or Alkylphenol Ethoxylate, and Rapeseed oil.

Another embodiment comprises cyclosporin A, Emcol CN-6®, and Rapeseed oil.

Another embodiment comprises cyclosporin A, Ethcarb®, and Rapeseed oil.

Another embodiment comprises cyclosporin A, Gemtex WNT-Conc®, and Rapeseed oil.

Another embodiment comprises cyclosporin A, Incrodet TD7-C®, and Rapeseed oil.

Another embodiment comprises cyclosporin A, Marlinat CM 105/80®, and Rapeseed oil.

Another embodiment comprises cyclosporin A, Marlowet 1072®, and Rapeseed oil.

Another embodiment comprises cyclosporin A, Marlowet 4530®, and Rapeseed oil.

Another embodiment comprises cyclosporin A, Marlowet 4530 LF®, and Rapeseed oil.

Another embodiment comprises cyclosporin A, Marlowet 4534®, and Rapeseed oil.

Another embodiment comprises cyclosporin A, Marlowet 4538®, and Rapeseed oil.

Another embodiment comprises cyclosporin A, Marlowet 4539®, and Rapeseed oil.

Another embodiment comprises cyclosporin A, Marlowet 4539 LF®, and Rapeseed oil.

Another embodiment comprises cyclosporin A, Marlowet 4541®, and Rapeseed oil.

Another embodiment comprises cyclosporin A, Miranate LEC-80®, and Rapeseed oil.

Another embodiment comprises cyclosporin A, Sandopan B®, and Rapeseed oil.

Another embodiment comprises cyclosporin A, Sandopan B Modified®, and Rapeseed oil.

Another embodiment comprises cyclosporin A, Sandopan LS-24 Gel®, and Rapeseed oil.

Another embodiment comprises cyclosporin A, Surfine T-A®, and Rapeseed oil.

Another embodiment comprises cyclosporin A, a Carboxylic Acid/Fatty Acid, and Rapeseed oil.

Another embodiment comprises cyclosporin A, Colaterge RAM®, and Rapeseed oil.

Another embodiment comprises cyclosporin A, Colatrope INC®, and Rapeseed oil.

Another embodiment comprises cyclosporin A, Crodacid B®, and Rapeseed oil.

Another embodiment comprises cyclosporin A, DeTrope CA-100®, and Rapeseed oil.

Another embodiment comprises cyclosporin A, Latol MTO®, and Rapeseed oil.

Another embodiment comprises cyclosporin A, Lumulse CC-33K®, and Rapeseed oil.

Another embodiment comprises cyclosporin A, Mulls 2218®, and Rapeseed oil.

Another embodiment comprises cyclosporin A, OL-600®, and Rapeseed oil.

Another embodiment comprises cyclosporin A, OL-800®, and Rapeseed oil.

Another embodiment comprises cyclosporin A, R-910®, and Rapeseed oil.

Another embodiment comprises cyclosporin A, S-210®, and Rapeseed oil.

Another embodiment comprises cyclosporin A, Sandopan DTC Acid®, and Rapeseed oil.

Another embodiment comprises cyclosporin A, Sandopan LS 24 N®, and Rapeseed oil.

Another embodiment comprises cyclosporin A, Sandopan MA-18®, and Rapeseed oil.

Another embodiment comprises cyclosporin A, an Ethoxylated Alcohol, and Rapeseed oil.

Another embodiment comprises cyclosporin A, Adsee 799®, and Rapeseed oil.

Another embodiment comprises cyclosporin A, Adsee 799®, and Rapeseed oil.

Another embodiment comprises cyclosporin A, Alfonic 610-3.5®, and Rapeseed oil.

Another embodiment comprises cyclosporin A, Alfonic 810-2®, and Rapeseed oil.

Another embodiment comprises cyclosporin A, Alfonic 810-6®, and Rapeseed oil.

Another embodiment comprises cyclosporin A, Alfonic 1012-3®, and Rapeseed oil.

Another embodiment comprises cyclosporin A, Alfonic 1012-5®, and Rapeseed oil.

Another embodiment comprises cyclosporin A, Alfonic 1216CO-1.5®, and Rapeseed oil.

Another embodiment comprises cyclosporin A, Alfonic 1216CO-7®, and Rapeseed oil.

Another embodiment comprises cyclosporin A, Alfonic 1412-3®, and Rapeseed oil.

Another embodiment comprises cyclosporin A, Alfonic 1412-7®, and Rapeseed oil.

Another embodiment comprises cyclosporin A, Arlasolve 200®, and Rapeseed oil.

Another embodiment comprises cyclosporin A, Arlasolve 200 Liquid®, and Rapeseed oil.

Another embodiment comprises cyclosporin A, Armix 180-C®, and Rapeseed oil.

Another embodiment comprises cyclosporin A, Armix 183®, and Rapeseed oil.

Another embodiment comprises cyclosporin A, Armul 2404®, and Rapeseed oil.

Another embodiment comprises cyclosporin A, Armul 2404®, and Rapeseed oil.

Another embodiment comprises cyclosporin A, Atlas EMJ-C®, and Rapeseed oil.

Another embodiment comprises cyclosporin A, Atlas G-2109®, and Rapeseed oil.

Another embodiment comprises cyclosporin A, Atlas G-3886®, and Rapeseed oil.

Another embodiment comprises cyclosporin A, Atlas G-3890®, and Rapeseed oil.

Another embodiment comprises cyclosporin A, Bio Soft E-20®, and Rapeseed oil.

Another embodiment comprises cyclosporin A, Bio Soft E-300®, and Rapeseed oil.

Another embodiment comprises cyclosporin A, Bio Soft E-400®, and Rapeseed oil.

Another embodiment comprises cyclosporin A, Bio Soft EN 600®, and Rapeseed oil.

Another embodiment comprises cyclosporin A, Bio Soft TD-400®, and Rapeseed oil.

Another embodiment comprises cyclosporin A, Bio Soft TD-630®, and Rapeseed oil.

Another embodiment comprises cyclosporin A, Brij 30®, and Rapeseed oil.

Another embodiment comprises cyclosporin A, Brij 52®, and Rapeseed oil.

Another embodiment comprises cyclosporin A, Brij 56®, and Rapeseed oil.

Another embodiment comprises cyclosporin A, Brij 58®, and Rapeseed oil.

Another embodiment comprises cyclosporin A, Brij 72®, and Rapeseed oil.

Another embodiment comprises cyclosporin A, Brij 76®, and Rapeseed oil.

Another embodiment comprises cyclosporin A, Brij 78®, and Rapeseed oil.

Another embodiment comprises cyclosporin A, Brij 93®, and Rapeseed oil.

Another embodiment comprises cyclosporin A, Brij 97®, and Rapeseed oil.

Another embodiment comprises cyclosporin A, Brij 98®, and Rapeseed oil.

Another embodiment comprises cyclosporin A, Brij 700®, and Rapeseed oil.

Another embodiment comprises cyclosporin A, Brij 700 S®, and Rapeseed oil.

Another embodiment comprises cyclosporin A, Brij 721®, and Rapeseed oil.

Another embodiment comprises cyclosporin A, Brij 721 S®, and Rapeseed oil.

Another embodiment comprises cyclosporin A, Burcoterge CDG®, and Rapeseed oil.

Another embodiment comprises cyclosporin A, Canasol AT 600®, and Rapeseed oil.

Another embodiment comprises cyclosporin A, Canasol AT 800®, and Rapeseed oil.

Another embodiment comprises cyclosporin A, Canasol AT 1200®, and Rapeseed oil.

Another embodiment comprises cyclosporin A, Canasol BJ 35®, and Rapeseed oil.

Another embodiment comprises cyclosporin A, Canasol BJ 36®, and Rapeseed oil.

Another embodiment comprises cyclosporin A, Canasol BJ 52®, and Rapeseed oil.

Another embodiment comprises cyclosporin A, Canasol BJ 58®, and Rapeseed oil.

Another embodiment comprises cyclosporin A, Canasol BJ 72®, and Rapeseed oil.

Another embodiment comprises cyclosporin A, Canasol BJ 78®, and Rapeseed oil.

Another embodiment comprises cyclosporin A, Canasol BJ 98®, and Rapeseed oil.

Another embodiment comprises cyclosporin A, Canasol BJ 307®, and Rapeseed oil.

Another embodiment comprises cyclosporin A, Cerfak 1400®, and Rapeseed oil.

Another embodiment comprises cyclosporin A, Cetomacrogol 1000 BP®, and Rapeseed oil.

Another embodiment comprises cyclosporin A, Chemonic C-2®, and Rapeseed oil.

Another embodiment comprises cyclosporin A, Chemonic C-10®, and Rapeseed oil.

Another embodiment comprises cyclosporin A, Chemonic C-20®, and Rapeseed oil.

Another embodiment comprises cyclosporin A, Chemonic CT-12®, and Rapeseed oil.

Another embodiment comprises cyclosporin A, Chemonic CT-20®, and Rapeseed oil.

Another embodiment comprises cyclosporin A, Chemonic CT-30®, and Rapeseed oil.

Another embodiment comprises cyclosporin A, Chemonic CT-55®, and Rapeseed oil.

Another embodiment comprises cyclosporin A, Chemonic G-7®, and Rapeseed oil.

Another embodiment comprises cyclosporin A, Chemonic G-26®, and Rapeseed oil.

Another embodiment comprises cyclosporin A, Chemonic L-4®, and Rapeseed oil.

Another embodiment comprises cyclosporin A, Chemonic L-7®, and Rapeseed oil.

Another embodiment comprises cyclosporin A, Chemonic L-12®, and Rapeseed oil.

Another embodiment comprises cyclosporin A, Chemonic L-23®, and Rapeseed oil.

Another embodiment comprises cyclosporin A, Chemonic O-2®, and Rapeseed oil.

Another embodiment comprises cyclosporin A, Chemonic O-5®, and Rapeseed oil.

Another embodiment comprises cyclosporin A, Chemonic O-10®, and Rapeseed oil.

Another embodiment comprises cyclosporin A, Chemonic O-20®, and Rapeseed oil.

Another embodiment comprises cyclosporin A, Chemonic S-2®, and Rapeseed oil.

Another embodiment comprises cyclosporin A, Chemonic S-10®, and Rapeseed oil.

Another embodiment comprises cyclosporin A, Chemonic S-20®, and Rapeseed oil.

Another embodiment comprises cyclosporin A, Colamulse FE®, and Rapeseed oil.

Another embodiment comprises cyclosporin A, Cremophor A 20®, and Rapeseed oil.

Another embodiment comprises cyclosporin A, Cremophor SA 2®, and Rapeseed oil.

Another embodiment comprises cyclosporin A, Dehydol 100®, and Rapeseed oil.

Another embodiment comprises cyclosporin A, Dehydol O-4®, and Rapeseed oil.

Another embodiment comprises cyclosporin A, Delonic C-18®, and Rapeseed oil.

Another embodiment comprises cyclosporin A, DeSonic 6T®, and Rapeseed oil.

Another embodiment comprises cyclosporin A, DeSonic 9D®, and Rapeseed oil.

Another embodiment comprises cyclosporin A, DeSonic 9T®, and Rapeseed oil.

Another embodiment comprises cyclosporin A, DeSonic 12D®, and Rapeseed oil.

Another embodiment comprises cyclosporin A, DeSonic 12T®, and Rapeseed oil.

Another embodiment comprises cyclosporin A, DeSonic 15T®, and Rapeseed oil.

Another embodiment comprises cyclosporin A, DeSonic TDA-9®, and Rapeseed oil.

Another embodiment comprises cyclosporin A, DeThox GLG-7®, and Rapeseed oil.

Another embodiment comprises cyclosporin A, DeThox GLG-26®, and Rapeseed oil.

Another embodiment comprises cyclosporin A, DeThox LA-4®, and Rapeseed oil.

Another embodiment comprises cyclosporin A, DeThox LA-23®, and Rapeseed oil.

Another embodiment comprises cyclosporin A, DeThox SA-80®, and Rapeseed oil.

Another embodiment comprises cyclosporin A, Disponil O5®, and Rapeseed oil.

Another embodiment comprises cyclosporin A, Eccoterge EO-41B®, and Rapeseed oil.

Another embodiment comprises cyclosporin A, Empilan KA2.5/90®, and Rapeseed oil.

Another embodiment comprises cyclosporin A, Empilan KA5/90®, and Rapeseed oil.

Another embodiment comprises cyclosporin A, Empilan KM-20®, and Rapeseed oil.

Another embodiment comprises cyclosporin A, Empilan KM-50®, and Rapeseed oil.

Another embodiment comprises cyclosporin A, Empilan L-23®, and Rapeseed oil.

Another embodiment comprises cyclosporin A, Ethylan 25-3®, and Rapeseed oil.

Another embodiment comprises cyclosporin A, Ethylan 1204®, and Rapeseed oil.

Another embodiment comprises cyclosporin A, Ethylan DA-4®, and Rapeseed oil.

Another embodiment comprises cyclosporin A, Ethylan LA-230®, and Rapeseed oil.

Another embodiment comprises cyclosporin A, Ethylan SN®, and Rapeseed oil.

Another embodiment comprises cyclosporin A, Ethylan TD-60®, and Rapeseed oil.

Another embodiment comprises cyclosporin A, Ethylan TD-100®, and Rapeseed oil.

Another embodiment comprises cyclosporin A, Ethylan TD-1407®, and Rapeseed oil.

Another embodiment comprises cyclosporin A, Eumulgin B1®, and Rapeseed oil.

Another embodiment comprises cyclosporin A, Eumulgin B2®, and Rapeseed oil.

Another embodiment comprises cyclosporin A, Eumulgin B3®, and Rapeseed oil.

Another embodiment comprises cyclosporin A, Eumulgin O-10®, and Rapeseed oil.

Another embodiment comprises cyclosporin A, Flo Mo 80/20®, and Rapeseed oil.

Another embodiment comprises cyclosporin A, Flo Mo Low Foam®, and Rapeseed oil.

Another embodiment comprises cyclosporin A, Forlan C-24®, and Rapeseed oil.

Another embodiment comprises cyclosporin A, Genapol 1454®, and Rapeseed oil.

Another embodiment comprises cyclosporin A, Genapol BA-020®, and Rapeseed oil.

Another embodiment comprises cyclosporin A, Genapol BA-040®, and Rapeseed oil.

Another embodiment comprises cyclosporin A, Genapol C-100®, and Rapeseed oil.

Another embodiment comprises cyclosporin A, Genapol DA 060®, and Rapeseed oil.

Another embodiment comprises cyclosporin A, Genapol HS 020®, and Rapeseed oil.

Another embodiment comprises cyclosporin A, Genapol HS 200®, and Rapeseed oil.

Another embodiment comprises cyclosporin A, Genapol ID-040®, and Rapeseed oil.

Another embodiment comprises cyclosporin A, Genapol ID-060®, and Rapeseed oil.

Another embodiment comprises cyclosporin A, Genapol ID-090®, and Rapeseed oil.

Another embodiment comprises cyclosporin A, Genapol LA 010®, and Rapeseed oil.

Another embodiment comprises cyclosporin A, Genapol LA 020®, and Rapeseed oil.

Another embodiment comprises cyclosporin A, Genapol LA 030®, and Rapeseed oil.

Another embodiment comprises cyclosporin A, Genapol LA 040®, and Rapeseed oil.

Another embodiment comprises cyclosporin A, Genapol LA 050®, and Rapeseed oil.

Another embodiment comprises cyclosporin A, Genapol LA 060®, and Rapeseed oil.

Another embodiment comprises cyclosporin A, Genapol LA 070®, and Rapeseed oil.

Another embodiment comprises cyclosporin A, Genapol LA 070S®, and Rapeseed oil.

Another embodiment comprises cyclosporin A, Genapol LA 230®, and Rapeseed oil.

Another embodiment comprises cyclosporin A, Genapol O 020®, and Rapeseed oil.

Another embodiment comprises cyclosporin A, Genapol O 050®, and Rapeseed oil.

Another embodiment comprises cyclosporin A, Genapol O 100®, and Rapeseed oil.

Another embodiment comprises cyclosporin A, Genapol O 200®, and Rapeseed oil.

Another embodiment comprises cyclosporin A, Genapol SA 030®, and Rapeseed oil.

Another embodiment comprises cyclosporin A, Genapol SA 120®, and Rapeseed oil.

Another embodiment comprises cyclosporin A, Genapol T-020®, and Rapeseed oil.

Another embodiment comprises cyclosporin A, Genapol UD-030®, and Rapeseed oil.

Another embodiment comprises cyclosporin A, Genapol UD-050®, and Rapeseed oil.

Another embodiment comprises cyclosporin A, Genapol UD-070®, and Rapeseed oil.

Another embodiment comprises cyclosporin A, Genapol UD-079®, and Rapeseed oil.

Another embodiment comprises cyclosporin A, Genapol UD-080®, and Rapeseed oil.

Another embodiment comprises cyclosporin A, Genapol UD-110®, and Rapeseed oil.

Another embodiment comprises cyclosporin A, Genapol X 030®, and Rapeseed oil.

Another embodiment comprises cyclosporin A, Genapol X 050®, and Rapeseed oil.

Another embodiment comprises cyclosporin A, Genapol X 060®, and Rapeseed oil.

Another embodiment comprises cyclosporin A, Genapol X 070®, and Rapeseed oil.

Another embodiment comprises cyclosporin A, Genapol X 080®, and Rapeseed oil.

Another embodiment comprises cyclosporin A, Genapol X 100®, and Rapeseed oil.

Another embodiment comprises cyclosporin A, Genapol X159®, and Rapeseed oil.

Another embodiment comprises cyclosporin A, Generol 122 E5®, and Rapeseed oil.

Another embodiment comprises cyclosporin A, Generol 122 E25®, and Rapeseed oil.

Another embodiment comprises cyclosporin A, Hostacerin T-3®, and Rapeseed oil.

Another embodiment comprises cyclosporin A, Iconol LF 110®, and Rapeseed oil.

Another embodiment comprises cyclosporin A, Incropol CS-20®, and Rapeseed oil.

Another embodiment comprises cyclosporin A, Lexemul CS-20®, and Rapeseed oil.

Another embodiment comprises cyclosporin A, Liponic EG-1®, and Rapeseed oil.

Another embodiment comprises cyclosporin A, Lipowax D®, and Rapeseed oil.

Another embodiment comprises cyclosporin A, Lipowax G®, and Rapeseed oil.

Another embodiment comprises cyclosporin A, Lipowax NI®, and Rapeseed oil.

Another embodiment comprises cyclosporin A, Lipowax P®, and Rapeseed oil.

Another embodiment comprises cyclosporin A, Lipowax P-31®, and Rapeseed oil.

Another embodiment comprises cyclosporin A, Lipowax PR®, and Rapeseed oil.

Another embodiment comprises cyclosporin A, Lumulse CS-20®, and Rapeseed oil.

Another embodiment comprises cyclosporin A, Macol CSA-20®, and Rapeseed oil.

Another embodiment comprises cyclosporin A, Marlox B 24/50®, and Rapeseed oil.

Another embodiment comprises cyclosporin A, Mazawet 77®, and Rapeseed oil.

Another embodiment comprises cyclosporin A, Norfoi 1713®, and Rapeseed oil.

Another embodiment comprises cyclosporin A, Norfox 2579®, and Rapeseed oil.

Another embodiment comprises cyclosporin A, Norfox Lo Foam®, and Rapeseed oil.

Another embodiment comprises cyclosporin A, Promulgen D®, and Rapeseed oil.

Another embodiment comprises cyclosporin A, Promulgen G®, and Rapeseed oil.

Another embodiment comprises cyclosporin A, Renex 30®, and Rapeseed oil.

Another embodiment comprises cyclosporin A, Renex 36®, and Rapeseed oil.

Another embodiment comprises cyclosporin A, Rhodasurf A 24®, and Rapeseed oil.

Another embodiment comprises cyclosporin A, Rhodasurf AAE-10®, and Rapeseed oil.

Another embodiment comprises cyclosporin A, Rhodasurf BEH-25®, and Rapeseed oil.

Another embodiment comprises cyclosporin A, Rhodasurf BEH-40®, and Rapeseed oil.

Another embodiment comprises cyclosporin A, Rhodasurf DA 530®, and Rapeseed oil.

Another embodiment comprises cyclosporin A, Rhodasurf DA 630®, and Rapeseed oil.

Another embodiment comprises cyclosporin A, Rhodasurf DA 639®, and Rapeseed oil.

Another embodiment comprises cyclosporin A, Rhodasurf LAN-23®, and Rapeseed oil.

Another embodiment comprises cyclosporin A, Rhodasurf ON-870®, and Rapeseed oil.

Another embodiment comprises cyclosporin A, Rhodasurf ON-877®, and Rapeseed oil.

Another embodiment comprises cyclosporin A, Rhodasurf TB-970 FLK®, and Rapeseed oil.

Another embodiment comprises cyclosporin A, Ritacet-20®, and Rapeseed oil.

Another embodiment comprises cyclosporin A, Ritachol 1000®, and Rapeseed oil.

Another embodiment comprises cyclosporin A, Ritachol 2000®, and Rapeseed oil.

Another embodiment comprises cyclosporin A, Ritachol 5000®, and Rapeseed oil.

Another embodiment comprises cyclosporin A, Ritox 35®, and Rapeseed oil.

Another embodiment comprises cyclosporin A, Surfonic DA-4®, and Rapeseed oil.

Another embodiment comprises cyclosporin A, Surfonic DA-6®, and Rapeseed oil.

Another embodiment comprises cyclosporin A, Surfonic L46-7®, and Rapeseed oil.

Another embodiment comprises cyclosporin A, Surfonic POA®, and Rapeseed oil.

Another embodiment comprises cyclosporin A, Synthrapol KB®, and Rapeseed oil.

Another embodiment comprises cyclosporin A, Teginacid®, and Rapeseed oil.

Another embodiment comprises cyclosporin A, Teginacid C®, and Rapeseed oil.

Another embodiment comprises cyclosporin A, Tegotens EC 11®, and Rapeseed oil.

Another embodiment comprises cyclosporin A, Tinegal NA®, and Rapeseed oil.

Another embodiment comprises cyclosporin A, Tomadol 400®, and Rapeseed oil.

Another embodiment comprises cyclosporin A, Tomadol 600®, and Rapeseed oil.

Another embodiment comprises cyclosporin A, Tomadol 900®, and Rapeseed oil.

Another embodiment comprises cyclosporin A, Uniperol O®, and Rapeseed oil.

Another embodiment comprises cyclosporin A, Witconol SN Series®, and Rapeseed oil.

Another embodiment comprises cyclosporin A, an Ethoxylated Alkylphenol, and Rapeseed oil.

Another embodiment comprises cyclosporin A, Antarox LF-222®, and Rapeseed oil.

Another embodiment comprises cyclosporin A, Atlox 775®, and Rapeseed oil.

Another embodiment comprises cyclosporin A, Caloxylate N-9®, and Rapeseed oil.

Another embodiment comprises cyclosporin A, Canasol NF-1000®, and Rapeseed oil.

Another embodiment comprises cyclosporin A, Canasol NF-3000®, and Rapeseed oil.

Another embodiment comprises cyclosporin A, Canasol NF-3070®, and Rapeseed oil.

Another embodiment comprises cyclosporin A, Canasol OF 1670®, and Rapeseed oil.

Another embodiment comprises cyclosporin A, Canasol OF 2570®, and Rapeseed oil.

Another embodiment comprises cyclosporin A, Canasol OF 4070®, and Rapeseed oil.

Another embodiment comprises cyclosporin A, Chemax DNP-8®, and Rapeseed oil.

Another embodiment comprises cyclosporin A, Chemax DNP-18®, and Rapeseed oil.

Another embodiment comprises cyclosporin A, Chemax DNP-150/50®, and Rapeseed oil.

Another embodiment comprises cyclosporin A, DeSonic 1.5N®, and Rapeseed oil.

Another embodiment comprises cyclosporin A, DeSonic 4N®, and Rapeseed oil.

Another embodiment comprises cyclosporin A, DeSonic 5N®, and Rapeseed oil.

Another embodiment comprises cyclosporin A, DeSonic 6D®, and Rapeseed oil.

Another embodiment comprises cyclosporin A, DeSonic 6N®, and Rapeseed oil.

Another embodiment comprises cyclosporin A, DeSonic 7N®, and Rapeseed oil.

Another embodiment comprises cyclosporin A, DeSonic 9N®, and Rapeseed oil.

Another embodiment comprises cyclosporin A, DeSonic 10D®, and Rapeseed oil.

Another embodiment comprises cyclosporin A, DeSonic 11N®, and Rapeseed oil.

Another embodiment comprises cyclosporin A, DeSonic 12N®, and Rapeseed oil.

Another embodiment comprises cyclosporin A, DeSonic 15N®, and Rapeseed oil.

Another embodiment comprises cyclosporin A, DeSonic 20N®, and Rapeseed oil.

Another embodiment comprises cyclosporin A, Eccoscour RC®, and Rapeseed oil.

Another embodiment comprises cyclosporin A, Eccoterge EO-100®, and Rapeseed oil.

Another embodiment comprises cyclosporin A, Emulsifier 632/90%®, and Rapeseed oil.

Another embodiment comprises cyclosporin A, Geronol AG-821®, and Rapeseed oil.

Another embodiment comprises cyclosporin A, Gradonic N-95®, and Rapeseed oil.

Another embodiment comprises cyclosporin A, Hetoxide NP-4®, and Rapeseed oil.

Another embodiment comprises cyclosporin A, Hetoxide NP-30®, and Rapeseed oil.

Another embodiment comprises cyclosporin A, Hostapal N-100®, and Rapeseed oil.

Another embodiment comprises cyclosporin A, Hostapal N-110®, and Rapeseed oil.

Another embodiment comprises cyclosporin A, Igepal CTA-639W®, and Rapeseed oil.

Another embodiment comprises cyclosporin A, Igepal DAP-9®, and Rapeseed oil.

Another embodiment comprises cyclosporin A, Igepal OD-410®, and Rapeseed oil.

Another embodiment comprises cyclosporin A, Igepal SS-837®, and Rapeseed oil.

Another embodiment comprises cyclosporin A, Lipocol NP-9 USP®, and Rapeseed oil.

Another embodiment comprises cyclosporin A, Macol DNP-10®, and Rapeseed oil.

Another embodiment comprises cyclosporin A, Marlophen NP 5®, and Rapeseed oil.

Another embodiment comprises cyclosporin A, Marlophen P 1®, and Rapeseed oil.

Another embodiment comprises cyclosporin A, Surfonic NB®, and Rapeseed oil.

Another embodiment comprises cyclosporin A, Sudonic OPB-307®, and Rapeseed oil.

Another embodiment comprises cyclosporin A, Surfonic OPB-407®, and Rapeseed oil.

Another embodiment comprises cyclosporin A, Syn Fac 334®, and Rapeseed oil.

Another embodiment comprises cyclosporin A, Syn Fac 8216®, and Rapeseed oil.

Another embodiment comprises cyclosporin A, Triton N-57®, and Rapeseed oil.

Another embodiment comprises cyclosporin A, Trycol 6956®, and Rapeseed oil.

Another embodiment comprises cyclosporin A, Trycol 6961®, and Rapeseed oil.

Another embodiment comprises cyclosporin A, Trycol 6964®, and Rapeseed oil.

Another embodiment comprises cyclosporin A, Trycol 6969®, and Rapeseed oil.

Another embodiment comprises cyclosporin A, Trycol 6974®, and Rapeseed oil.

Another embodiment comprises cyclosporin A, Witbreak DRB-127®, and Rapeseed oil.

Another embodiment comprises cyclosporin A, Witbreak DRB-127®, and Rapeseed oil.

Another embodiment comprises cyclosporin A, Witconol NP Series®, and Rapeseed oil.

Another embodiment comprises cyclosporin A, an Ethoxylated Aryl Phenol, and Rapeseed oil.

Another embodiment comprises cyclosporin A, Soprophor BSU®, and Rapeseed oil.

Another embodiment comprises cyclosporin A, Soprophor CY/8®, and Rapeseed oil.

Another embodiment comprises cyclosporin A, Soprophor S/25®, and Rapeseed oil.

Another embodiment comprises cyclosporin A, Witconol NIO®, and Rapeseed oil.

Another embodiment comprises cyclosporin A, Witconol NIW®, and Rapeseed oil.

Another embodiment comprises cyclosporin A, Witconol S-100®, and Rapeseed oil.

Another embodiment comprises cyclosporin A, an Ethoxylated Fatty Acid, and Rapeseed oil.

Another embodiment comprises cyclosporin A, Aldo PGHMS®, and Rapeseed oil.

Another embodiment comprises cyclosporin A, Alkamuls TO-15/HR®, and Rapeseed oil.

Another embodiment comprises cyclosporin A, Armotan AL-69-66®, and Rapeseed oil.

Another embodiment comprises cyclosporin A, Cerasynt 840®, and Rapeseed oil.

Another embodiment comprises cyclosporin A, Cerasynt 945®, and Rapeseed oil.

Another embodiment comprises cyclosporin A, Crystal Inhibitor No. 5®, and Rapeseed oil.

Another embodiment comprises cyclosporin A, DeThox Acid L-9®, and Rapeseed oil.

Another embodiment comprises cyclosporin A, DeThox Acid S-8®, and Rapeseed oil.

Another embodiment comprises cyclosporin A, Ethofat 242/25®, and Rapeseed oil.

Another embodiment comprises cyclosporin A, Hydropalat 65®, and Rapeseed oil.

Another embodiment comprises cyclosporin A, Lipo EGMS®, and Rapeseed oil.

Another embodiment comprises cyclosporin A, Lipopeg 2 DL®, and Rapeseed oil.

Another embodiment comprises cyclosporin A, Lipopeg 4 DL®, and Rapeseed oil.

Another embodiment comprises cyclosporin A, Lipopeg 4-L®, and Rapeseed oil.

Another embodiment comprises cyclosporin A, Lipopeg 39-S®, and Rapeseed oil.

Another embodiment comprises cyclosporin A, Lipopeg 4-S®, and Rapeseed oil.

Another embodiment comprises cyclosporin A, Lipopeg 10-S®, and Rapeseed oil.

Another embodiment comprises cyclosporin A, Lipopeg 100-S®, and Rapeseed oil.

Another embodiment comprises cyclosporin A, Lipopeg 6000 DS®, and Rapeseed oil.

Another embodiment comprises cyclosporin A, Lumulse 40-L®, and Rapeseed oil.

Another embodiment comprises cyclosporin A, Lumulse 40-S®, and Rapeseed oil.

Another embodiment comprises cyclosporin A, Lumulse 42-L®, and Rapeseed oil.

Another embodiment comprises cyclosporin A, Lumulse 42-S®, and Rapeseed oil.

Another embodiment comprises cyclosporin A, Lumulse 100-S®, and Rapeseed oil.

Another embodiment comprises cyclosporin A, Lumulse 602-S®, and Rapeseed oil.

Another embodiment comprises cyclosporin A, Magrabar PGE-20-0®, and Rapeseed oil.

Another embodiment comprises cyclosporin A, Magrabar PGE-20L®, and Rapeseed oil.

Another embodiment comprises cyclosporin A, Magrabar PGE-20T®, and Rapeseed oil.

Another embodiment comprises cyclosporin A, Magrabar PGE-22-0®, and Rapeseed oil.

Another embodiment comprises cyclosporin A, Magrabar PGE-22L®, and Rapeseed oil.

Another embodiment comprises cyclosporin A, Magrabar PGE-22T®, and Rapeseed oil.

Another embodiment comprises cyclosporin A, Magrabar PGE-40-0®, and Rapeseed oil.

Another embodiment comprises cyclosporin A, Magrabar PGE-40L®, and Rapeseed oil.

Another embodiment comprises cyclosporin A, Magrabar PGE-40T®, and Rapeseed oil.

Another embodiment comprises cyclosporin A, Magrabar PGE-42-0®, and Rapeseed oil.

Another embodiment comprises cyclosporin A, Magrabar PGE-42L®, and Rapeseed oil.

Another embodiment comprises cyclosporin A, Magrabar PGE-42T®, and Rapeseed oil.

Another embodiment comprises cyclosporin A, Magrabar PGE-60-0®, and Rapeseed oil.

Another embodiment comprises cyclosporin A, Magrabar PGE-60L®, and Rapeseed oil.

Another embodiment comprises cyclosporin A, Magrabar PGE-60T®, and Rapeseed oil.

Another embodiment comprises cyclosporin A, Magrabar PGE-62-0®, and Rapeseed oil.

Another embodiment comprises cyclosporin A, Magrabar PGE-62L®, and Rapeseed oil.

Another embodiment comprises cyclosporin A, Magrabar PGE-62T®, and Rapeseed oil.

Another embodiment comprises cyclosporin A, Mapeg S-40K®, and Rapeseed oil.

Another embodiment comprises cyclosporin A, Marlowet OTS®, and Rapeseed oil.

Another embodiment comprises cyclosporin A, Naturechem PGR®, and Rapeseed oil.

Another embodiment comprises cyclosporin A, PG No. 4®, and Rapeseed oil.

Another embodiment comprises cyclosporin A, Renex 20®, and Rapeseed oil.

Another embodiment comprises cyclosporin A, Ritox 52®, and Rapeseed oil.

Another embodiment comprises cyclosporin A, Ritox 53®, and Rapeseed oil.

Another embodiment comprises cyclosporin A, Ritox 59®, and Rapeseed oil.

Another embodiment comprises cyclosporin A, Surfax 8916/A®, and Rapeseed oil.

Another embodiment comprises cyclosporin A, Tego Acid S 40 P®, and Rapeseed oil.

Another embodiment comprises cyclosporin A, Tego Acid S 100 P®, and Rapeseed oil.

Another embodiment comprises cyclosporin A, Tween 20®, and Rapeseed oil.

Another embodiment comprises cyclosporin A, Volpo 131®, and Rapeseed oil.

Another embodiment comprises cyclosporin A, an Ethoxylated Fatty Ester or Oil, and Rapeseed oil.

Another embodiment comprises cyclosporin A, Acconon 6-C10®, and Rapeseed oil.

Another embodiment comprises cyclosporin A, Acconon CC-6®, and Rapeseed oil.

Another embodiment comprises cyclosporin A, Acconon CO-7®, and Rapeseed oil.

Another embodiment comprises cyclosporin A, Aldosperse 40/60 FG®, and Rapeseed oil.

Another embodiment comprises cyclosporin A, Aldosperse ML-23®, and Rapeseed oil.

Another embodiment comprises cyclosporin A, Aldosperse MS-20 FG®, and Rapeseed oil.

Another embodiment comprises cyclosporin A, Alkamuls EL-620®, and Rapeseed oil.

Another embodiment comprises cyclosporin A, Alkamuls EL-719®, and Rapeseed oil.

Another embodiment comprises cyclosporin A, Alkamuls EL-985®, and Rapeseed oil.

Another embodiment comprises cyclosporin A, Arlatone G®, and Rapeseed oil.

Another embodiment comprises cyclosporin A, Arlatone T®, and Rapeseed oil.

Another embodiment comprises cyclosporin A, Atlas G-1045A®, and Rapeseed oil.

Another embodiment comprises cyclosporin A, Atlas G-1086®, and Rapeseed oil.

Another embodiment comprises cyclosporin A, Atlas G-1087®, and Rapeseed oil.

Another embodiment comprises cyclosporin A, Atlas G-1089®, and Rapeseed oil.

Another embodiment comprises cyclosporin A, Atlas G-1096®, and Rapeseed oil.

Another embodiment comprises cyclosporin A, Atlas G-1292®, and Rapeseed oil.

Another embodiment comprises cyclosporin A, Atlas G-1293®, and Rapeseed oil.

Another embodiment comprises cyclosporin A, Atlas G-1300®, and Rapeseed oil.

Another embodiment comprises cyclosporin A, Atlas G-7076®, and Rapeseed oil.

Another embodiment comprises cyclosporin A, Capmul EMG®, and Rapeseed oil.

Another embodiment comprises cyclosporin A, Chemonic CO-40®, and Rapeseed oil.

Another embodiment comprises cyclosporin A, Chemonic LI-3®, and Rapeseed oil.

Another embodiment comprises cyclosporin A, Chemonic LI-7®, and Rapeseed oil.

Another embodiment comprises cyclosporin A, Cirrasol GM®, and Rapeseed oil.

Another embodiment comprises cyclosporin A, Cremophor CO 40®, and Rapeseed oil.

Another embodiment comprises cyclosporin A, Cremophor CO 410®, and Rapeseed oil.

Another embodiment comprises cyclosporin A, Cremophor EL®, and Rapeseed oil.

Another embodiment comprises cyclosporin A, Cremophor GC7®, and Rapeseed oil.

Another embodiment comprises cyclosporin A, Cremophor RH-40®, and Rapeseed oil.

Another embodiment comprises cyclosporin A, Crovol A-40®, and Rapeseed oil.

Another embodiment comprises cyclosporin A, Crovol A-70®, and Rapeseed oil.

Another embodiment comprises cyclosporin A, Crovol M-70®, and Rapeseed oil.

Another embodiment comprises cyclosporin A, Crovol PK-70®, and Rapeseed oil.

Another embodiment comprises cyclosporin A, Cutina E-24®, and Rapeseed oil.

Another embodiment comprises cyclosporin A, Dacospin 12-R®, and Rapeseed oil.

Another embodiment comprises cyclosporin A, Dehymuls HRE-7®, and Rapeseed oil.

Another embodiment comprises cyclosporin A, DeSonic 30C®, and Rapeseed oil.

Another embodiment comprises cyclosporin A, DeSonic 36C®, and Rapeseed oil.

Another embodiment comprises cyclosporin A, DeSonic 40C®, and Rapeseed oil.

Another embodiment comprises cyclosporin A, Durfax 60®, and Rapeseed oil.

Another embodiment comprises cyclosporin A, Durfax 65®, and Rapeseed oil.

Another embodiment comprises cyclosporin A, Durfax 80®, and Rapeseed oil.

Another embodiment comprises cyclosporin A, Durfax EOM®, and Rapeseed oil.

Another embodiment comprises cyclosporin A, Eccoterge NF-2®, and Rapeseed oil.

Another embodiment comprises cyclosporin A, Emulpon CO-360®, and Rapeseed oil.

Another embodiment comprises cyclosporin A, Emulpon CO-550®, and Rapeseed oil.

Another embodiment comprises cyclosporin A, Emulsogen EL®, and Rapeseed oil.

Another embodiment comprises cyclosporin A, Emulsogen HCO 040®, and Rapeseed oil.

Another embodiment comprises cyclosporin A, Emulsogen HCO 060®, and Rapeseed oil.

Another embodiment comprises cyclosporin A, Emulsynt 1055®, and Rapeseed oil.

Another embodiment comprises cyclosporin A, Ethox 3095®, and Rapeseed oil.

Another embodiment comprises cyclosporin A, Eumulgin RO-40®, and Rapeseed oil.

Another embodiment comprises cyclosporin A, Genapol G-260®, and Rapeseed oil.

Another embodiment comprises cyclosporin A, Glycosperse L-20®, and Rapeseed oil.

Another embodiment comprises cyclosporin A, Glycosperse O-5®, and Rapeseed oil.

Another embodiment comprises cyclosporin A, Glycosperse O-20®, and Rapeseed oil.

Another embodiment comprises cyclosporin A, Glycosperse O-20 FG®, and Rapeseed oil.

Another embodiment comprises cyclosporin A, Glycosperse S-20®, and Rapeseed oil.

Another embodiment comprises cyclosporin A, Glycosperse S-20 FG®, and Rapeseed oil.

Another embodiment comprises cyclosporin A, Glycosperse TS-20®, and Rapeseed oil.

Another embodiment comprises cyclosporin A, Glycosperse TS-20 FG®, and Rapeseed oil.

Another embodiment comprises cyclosporin A, Hetan SL®, and Rapeseed oil.

Another embodiment comprises cyclosporin A, Hetan SO®, and Rapeseed oil.

Another embodiment comprises cyclosporin A, Hetan SS®, and Rapeseed oil.

Another embodiment comprises cyclosporin A, Hetoxide C-2®, and Rapeseed oil.

Another embodiment comprises cyclosporin A, Hetoxide C-9®, and Rapeseed oil.

Another embodiment comprises cyclosporin A, Hetoxide C-15®, and Rapeseed oil.

Another embodiment comprises cyclosporin A, Hetoxide C-25®, and Rapeseed oil.

Another embodiment comprises cyclosporin A, Hetoxide C-40®, and Rapeseed oil.

Another embodiment comprises cyclosporin A, Hetoxide C-200®, and Rapeseed oil.

Another embodiment comprises cyclosporin A, Hetoxide C-200-50%®, and Rapeseed oil.

Another embodiment comprises cyclosporin A, Hetoxide GC-30®, and Rapeseed oil.

Another embodiment comprises cyclosporin A, Hetoxide HC-60®, and Rapeseed oil.

Another embodiment comprises cyclosporin A, Ice No. 2®, and Rapeseed oil.

Another embodiment comprises cyclosporin A, Incrocas 30/40®, and Rapeseed oil.

Another embodiment comprises cyclosporin A, Lexol EC®, and Rapeseed oil.

Another embodiment comprises cyclosporin A, Lexol EO®, and Rapeseed oil.

Another embodiment comprises cyclosporin A, Lipocol HCO-40®, and Rapeseed oil.

Another embodiment comprises cyclosporin A, Lipocol HCO-60®, and Rapeseed oil.

Another embodiment comprises cyclosporin A, Lipocol O-3 Special®, and Rapeseed oil.

Another embodiment comprises cyclosporin A, Lipopeg 2-L®, and Rapeseed oil.

Another embodiment comprises cyclosporin A, Lipopeg 4-DO®, and Rapeseed oil.

Another embodiment comprises cyclosporin A, Lipopeg 4-DS®, and Rapeseed oil.

Another embodiment comprises cyclosporin A, Lipovol GTB®, and Rapeseed oil.

Another embodiment comprises cyclosporin A, Lonzest SML-20®, and Rapeseed oil.

Another embodiment comprises cyclosporin A, Lonzest SMO-20®, and Rapeseed oil.

Another embodiment comprises cyclosporin A, Lonzest SMS-20®, and Rapeseed oil.

Another embodiment comprises cyclosporin A, Lonzest STO-20®, and Rapeseed oil.

Another embodiment comprises cyclosporin A, Lonzest STS-20®, and Rapeseed oil.

Another embodiment comprises cyclosporin A, Lumulse GR-40®, and Rapeseed oil.

Another embodiment comprises cyclosporin A, Lumulse GRH-40®, and Rapeseed oil.

Another embodiment comprises cyclosporin A, Lumulse POE (7) GML®, and Rapeseed oil.

Another embodiment comprises cyclosporin A, Lumulse POE (12) Glyc®, and Rapeseed oil.

Another embodiment comprises cyclosporin A, Lumulse POE (40) MS KP®, and Rapeseed oil.

Another embodiment comprises cyclosporin A, Marlowet 4750®, and Rapeseed oil.

Another embodiment comprises cyclosporin A, Marlowet LVS®, and Rapeseed oil.

Another embodiment comprises cyclosporin A, Marlowet R 11®, and Rapeseed oil.

Another embodiment comprises cyclosporin A, Marlowet R 40®, and Rapeseed oil.

Another embodiment comprises cyclosporin A, Mazol 80 MGK®, and Rapeseed oil.

Another embodiment comprises cyclosporin A, Nonionic Emulsifier T-9®, and Rapeseed oil.

Another embodiment comprises cyclosporin A, Oronal LCG®, and Rapeseed oil.

Another embodiment comprises cyclosporin A, Polyderm PPI-CO-200®, and Rapeseed oil.

Another embodiment comprises cyclosporin A, Polyderm PPI-CO-40®, and Rapeseed oil.

Another embodiment comprises cyclosporin A, Rewoderm LI 520-70®, and Rapeseed oil.

Another embodiment comprises cyclosporin A, Ritapeg 150 DS®, and Rapeseed oil.

Another embodiment comprises cyclosporin A, Softigen 767®, and Rapeseed oil.

Another embodiment comprises cyclosporin A, Surfactol 318®, and Rapeseed oil.

Another embodiment comprises cyclosporin A, Surfactol 365®, and Rapeseed oil.

Another embodiment comprises cyclosporin A, Syn Lube 107®, and Rapeseed oil.

Another embodiment comprises cyclosporin A, Syn Lube 728®, and Rapeseed oil.

Another embodiment comprises cyclosporin A, Syn Lube 1632H®, and Rapeseed oil.

Another embodiment comprises cyclosporin A, Syn Lube 6277-A®, and Rapeseed oil.

Another embodiment comprises cyclosporin A, T-Det C-20®, and Rapeseed oil.

Another embodiment comprises cyclosporin A, T-Det C-40®, and Rapeseed oil.

Another embodiment comprises cyclosporin A, Tally 100 Plus®, and Rapeseed oil.

Another embodiment comprises cyclosporin A, Uniperol EL®, and Rapeseed oil.

Another embodiment comprises cyclosporin A, a Fatty Ester, and Rapeseed oil.

Another embodiment comprises cyclosporin A, Actralube-Syn 147®, and Rapeseed oil.

Another embodiment comprises cyclosporin A, Atlas G-1556®, and Rapeseed oil.

Another embodiment comprises cyclosporin A, Atlas G-1564®, and Rapeseed oil.

Another embodiment comprises cyclosporin A, Atlasol Base Oil S®, and Rapeseed oil.

Another embodiment comprises cyclosporin A, Base ML®, and Rapeseed oil.

Another embodiment comprises cyclosporin A, Base MT®, and Rapeseed oil.

Another embodiment comprises cyclosporin A, Cerasynt 303®, and Rapeseed oil.

Another embodiment comprises cyclosporin A, Dermol 1012®, and Rapeseed oil.

Another embodiment comprises cyclosporin A, Kemester 4000®, and Rapeseed oil.

Another embodiment comprises cyclosporin A, Lactipol S®, and Rapeseed oil.

Another embodiment comprises cyclosporin A, Magrabar PGO®, and Rapeseed oil.

Another embodiment comprises cyclosporin A, Mayco Base BFO®, and Rapeseed oil.

Another embodiment comprises cyclosporin A, Methyl Linoleate®, and Rapeseed oil.

Another embodiment comprises cyclosporin A, Pationic 122A®, and Rapeseed oil.

Another embodiment comprises cyclosporin A, Pationic 138C®, and Rapeseed oil.

Another embodiment comprises cyclosporin A, Pationic CSL®, and Rapeseed oil.

Another embodiment comprises cyclosporin A, Pationic ISL®, and Rapeseed oil.

Another embodiment comprises cyclosporin A, Pationic SBL®, and Rapeseed oil.

Another embodiment comprises cyclosporin A, Pationic SSL®, and Rapeseed oil.

Another embodiment comprises cyclosporin A, Ritasol®, and Rapeseed oil.

Another embodiment comprises cyclosporin A, Tego Alkanol CS 20®, and Rapeseed oil.

Another embodiment comprises cyclosporin A, Tego Alkanol L23 P®, and Rapeseed oil.

Another embodiment comprises cyclosporin A, Tego Alkanol S2®, and Rapeseed oil.

Another embodiment comprises cyclosporin A, Tego Alkanol S20 P®, and Rapeseed oil.

Another embodiment comprises cyclosporin A, Triemulsitier 600 MS®, and Rapeseed oil.

Another embodiment comprises cyclosporin A, a Glycerol Esters, and Rapeseed oil.

Another embodiment comprises cyclosporin A, Agro #9 Wint SBO®, and Rapeseed oil.

Another embodiment comprises cyclosporin A, Ahcovel Base 700®, and Rapeseed oil.

Another embodiment comprises cyclosporin A, Aldo HMS FG®, and Rapeseed oil.

Another embodiment comprises cyclosporin A, Aldo MLD®, and Rapeseed oil.

Another embodiment comprises cyclosporin A, Aldo MLD FG®, and Rapeseed oil.

Another embodiment comprises cyclosporin A, Aldo MO FG®, and Rapeseed oil.

Another embodiment comprises cyclosporin A, Aldo MS®, and Rapeseed oil.

Another embodiment comprises cyclosporin A, Aldo MS FG®, and Rapeseed oil.

Another embodiment comprises cyclosporin A, Aldo MS LG FG®, and Rapeseed oil.

Another embodiment comprises cyclosporin A, Aldo MSD®, and Rapeseed oil.

Another embodiment comprises cyclosporin A, Aldo MSD FG®, and Rapeseed oil.

Another embodiment comprises cyclosporin A, Aldosperse O-20 FG®, and Rapeseed oil.

Another embodiment comprises cyclosporin A, Aldosperse TS-20 FG®, and Rapeseed oil.

Another embodiment comprises cyclosporin A, Aldosperse TS-40 FG®, and Rapeseed oil.

Another embodiment comprises cyclosporin A, Arlacel 165®, and Rapeseed oil.

Another embodiment comprises cyclosporin A, Arlacel 186®, and Rapeseed oil.

Another embodiment comprises cyclosporin A, Capmul GMO®, and Rapeseed oil.

Another embodiment comprises cyclosporin A, Capmul GMS®, and Rapeseed oil.

Another embodiment comprises cyclosporin A, Caprol 3GO®, and Rapeseed oil.

Another embodiment comprises cyclosporin A, Caprol 3GVS®, and Rapeseed oil.

Another embodiment comprises cyclosporin A, Caprol 6G2S®, and Rapeseed oil.

Another embodiment comprises cyclosporin A, Caprol 10G40®, and Rapeseed oil.

Another embodiment comprises cyclosporin A, Caprol 10G100®, and Rapeseed oil.

Another embodiment comprises cyclosporin A, Caprol ET®, and Rapeseed oil.

Another embodiment comprises cyclosporin A, Caprol PGE860®, and Rapeseed oil.

Another embodiment comprises cyclosporin A, Cerasynt 945®, and Rapeseed oil.

Another embodiment comprises cyclosporin A, Cerasynt GMS®, and Rapeseed oil.

Another embodiment comprises cyclosporin A, Cerasynt Q®, and Rapeseed oil.

Another embodiment comprises cyclosporin A, Cerasynt SD®, and Rapeseed oil.

Another embodiment comprises cyclosporin A, Cerasynt WM®, and Rapeseed oil.

Another embodiment comprises cyclosporin A, Chemsperse 14®, and Rapeseed oil.

Another embodiment comprises cyclosporin A, Cremophor GO-32®, and Rapeseed oil.

Another embodiment comprises cyclosporin A, Cremophor GS11®, and Rapeseed oil.

Another embodiment comprises cyclosporin A, Cremophor GS-32®, and Rapeseed oil.

Another embodiment comprises cyclosporin A, Cutina KD-16®, and Rapeseed oil.

Another embodiment comprises cyclosporin A, Dehymuls PGPH®, and Rapeseed oil.

Another embodiment comprises cyclosporin A, Dermol DODIS®, and Rapeseed oil.

Another embodiment comprises cyclosporin A, Dermol DOMIS®, and Rapeseed oil.

Another embodiment comprises cyclosporin A, Dermol G-76®, and Rapeseed oil.

Another embodiment comprises cyclosporin A, Dermol G-7DI®, and Rapeseed oil.

Another embodiment comprises cyclosporin A, Dermol NGDI®, and Rapeseed oil.

Another embodiment comprises cyclosporin A, Dermolan GLH®, and Rapeseed oil.

Another embodiment comprises cyclosporin A, Drewmulse GMO®, and Rapeseed oil.

Another embodiment comprises cyclosporin A, Drewpol 3-5-M®, and Rapeseed oil.

Another embodiment comprises cyclosporin A, Durlac 100 W®, and Rapeseed oil.

Another embodiment comprises cyclosporin A, Dur-Lo®, and Rapeseed oil.

Another embodiment comprises cyclosporin A, Dynasan 118®, and Rapeseed oil.

Another embodiment comprises cyclosporin A, EC-25®, and Rapeseed oil.

Another embodiment comprises cyclosporin A, EM 40®, and Rapeseed oil.

Another embodiment comprises cyclosporin A, Emerest 2400®, and Rapeseed oil.

Another embodiment comprises cyclosporin A, Emerest 2452®, and Rapeseed oil.

Another embodiment comprises cyclosporin A, Empilan G-26®, and Rapeseed oil.

Another embodiment comprises cyclosporin A, Genapol TSM®, and Rapeseed oil.

Another embodiment comprises cyclosporin A, Hostacerin DGI®, and Rapeseed oil.

Another embodiment comprises cyclosporin A, Hostacerin DGL®, and Rapeseed oil.

Another embodiment comprises cyclosporin A, Hostacerin DGMS®, and Rapeseed oil.

Another embodiment comprises cyclosporin A, Hostacerin DGSB®, and Rapeseed oil.

Another embodiment comprises cyclosporin A, Ice No. 2®, and Rapeseed oil.

Another embodiment comprises cyclosporin A, Imwitor 742®, and Rapeseed oil.

Another embodiment comprises cyclosporin A, Imwitor 780 K®, and Rapeseed oil.

Another embodiment comprises cyclosporin A, Imwitor 960 Flakes®, and Rapeseed oil.

Another embodiment comprises cyclosporin A, Isolan GI 34®, and Rapeseed oil.

Another embodiment comprises cyclosporin A, Isolan GO 33®, and Rapeseed oil.

Another embodiment comprises cyclosporin A, Kemester 1000®, and Rapeseed oil.

Another embodiment comprises cyclosporin A, Kemester 2000®, and Rapeseed oil.

Another embodiment comprises cyclosporin A, Kemester 2000®, and Rapeseed oil.

Another embodiment comprises cyclosporin A, Kemester 6000SE®, and Rapeseed oil.

Another embodiment comprises cyclosporin A, Lamecreme DOE 18®, and Rapeseed oil.

Another embodiment comprises cyclosporin A, Lexemul 515®, and Rapeseed oil.

Another embodiment comprises cyclosporin A, Lexemul 561®, and Rapeseed oil.

Another embodiment comprises cyclosporin A, Lexemul AR®, and Rapeseed oil.

Another embodiment comprises cyclosporin A, Lexemul AS®, and Rapeseed oil.

Another embodiment comprises cyclosporin A, Lexemul GDL®, and Rapeseed oil.

Another embodiment comprises cyclosporin A, Lexemul T®, and Rapeseed oil.

Another embodiment comprises cyclosporin A, Lipomulse 165®, and Rapeseed oil.

Another embodiment comprises cyclosporin A, Lumulse GML K®, and Rapeseed oil.

Another embodiment comprises cyclosporin A, Lumulse GMO K®, and Rapeseed oil.

Another embodiment comprises cyclosporin A, Lumulse GMR K®, and Rapeseed oil.

Another embodiment comprises cyclosporin A, Lumulse GMT K®, and Rapeseed oil.

Another embodiment comprises cyclosporin A, Magrabar GMC®, and Rapeseed oil.

Another embodiment comprises cyclosporin A, Magrabar GMO-CK®, and Rapeseed oil.

Another embodiment comprises cyclosporin A, Magrabar MDG-5050®, and Rapeseed oil.

Another embodiment comprises cyclosporin A, Magrabar PGO-315®, and Rapeseed oil.

Another embodiment comprises cyclosporin A, Magrabar PGO-1010®, and Rapeseed oil.

Another embodiment comprises cyclosporin A, Mazol 300K®, and Rapeseed oil.

Another embodiment comprises cyclosporin A, Mazol GMO-K®, and Rapeseed oil.

Another embodiment comprises cyclosporin A, Mazol GMS-K®, and Rapeseed oil.

Another embodiment comprises cyclosporin A, Mazol PG031-K®, and Rapeseed oil.

Another embodiment comprises cyclosporin A, Miglyol 812®, and Rapeseed oil.

Another embodiment comprises cyclosporin A, Norfox 165C®, and Rapeseed oil.

Another embodiment comprises cyclosporin A, Schercemol GMIS®, and Rapeseed oil.

Another embodiment comprises cyclosporin A, Tegin®, and Rapeseed oil.

Another embodiment comprises cyclosporin A, Tegin 4100 Pellets®, and Rapeseed oil.

Another embodiment comprises cyclosporin A, Tegin M Pellets®, and Rapeseed oil.

Another embodiment comprises cyclosporin A, Tegin OV®, and Rapeseed oil.

Another embodiment comprises cyclosporin A, Teginacid H®, and Rapeseed oil.

Another embodiment comprises cyclosporin A, Tego Cosmo P813®, and Rapeseed oil.

Another embodiment comprises cyclosporin A, Wickenol 535®, and Rapeseed oil.

Another embodiment comprises cyclosporin A, Witconol 14®, and Rapeseed oil.

Another embodiment comprises cyclosporin A, Witconol 14®, and Rapeseed oil.

Another embodiment comprises cyclosporin A, Witconol 14F®, and Rapeseed oil.

Another embodiment comprises cyclosporin A, Witconol 18L®, and Rapeseed oil.

Another embodiment comprises cyclosporin A, Witconol GOT®, and Rapeseed oil.

Another embodiment comprises cyclosporin A, Witconol MST®, and Rapeseed oil.

Another embodiment comprises cyclosporin A, Witconol RHT®, and Rapeseed oil.

Another embodiment comprises cyclosporin A, a Glycol Ester, and Rapeseed oil.

Another embodiment comprises cyclosporin A, Alkamuls 600 DO®, and Rapeseed oil.

Another embodiment comprises cyclosporin A, Alkamuls SEG®, and Rapeseed oil.

Another embodiment comprises cyclosporin A, Atlas EM-2®, and Rapeseed oil.

Another embodiment comprises cyclosporin A, Cerasynt IP®, and Rapeseed oil.

Another embodiment comprises cyclosporin A, Cerasynt M®, and Rapeseed oil.

Another embodiment comprises cyclosporin A, Cerasynt MN®, and Rapeseed oil.

Another embodiment comprises cyclosporin A, Cerasynt PA®, and Rapeseed oil.

Another embodiment comprises cyclosporin A, Chemsperse EGDS®, and Rapeseed oil.

Another embodiment comprises cyclosporin A, Chemsperse EGMS®, and Rapeseed oil.

Another embodiment comprises cyclosporin A, Colonial Monolaurin®, and Rapeseed oil.

Another embodiment comprises cyclosporin A, DeMuls SGE-95®, and Rapeseed oil.

Another embodiment comprises cyclosporin A, Eccoterge 200®, and Rapeseed oil.

Another embodiment comprises cyclosporin A, Emerest 2380®, and Rapeseed oil.

Another embodiment comprises cyclosporin A, Ethox 2610®, and Rapeseed oil.

Another embodiment comprises cyclosporin A, Ethox DO-9®, and Rapeseed oil.

Another embodiment comprises cyclosporin A, Ethox DO-14®, and Rapeseed oil.

Another embodiment comprises cyclosporin A, Ethox SO-9®, and Rapeseed oil.

Another embodiment comprises cyclosporin A, Fizul MD-318®, and Rapeseed oil.

Another embodiment comprises cyclosporin A, Genapol EGDS-VHP®, and Rapeseed oil.

Another embodiment comprises cyclosporin A, Genapol TS Powder®, and Rapeseed oil.

Another embodiment comprises cyclosporin A, Hostacerin WO®, and Rapeseed oil.

Another embodiment comprises cyclosporin A, Inversol 140®, and Rapeseed oil.

Another embodiment comprises cyclosporin A, Kemester 104®, and Rapeseed oil.

Another embodiment comprises cyclosporin A, Kemester 205®, and Rapeseed oil.

Another embodiment comprises cyclosporin A, Kemester 226®, and Rapeseed oil.

Another embodiment comprises cyclosporin A, Kemester 5221SE®, and Rapeseed oil.

Another embodiment comprises cyclosporin A, Kemester EGDS®, and Rapeseed oil.

Another embodiment comprises cyclosporin A, Lexemul EGDS®, and Rapeseed oil.

Another embodiment comprises cyclosporin A, Lexemul EGMS®, and Rapeseed oil.

Another embodiment comprises cyclosporin A, Lexemul P®, and Rapeseed oil.

Another embodiment comprises cyclosporin A, Lipo DGLS®, and Rapeseed oil.

Another embodiment comprises cyclosporin A, Lipo EGDS®, and Rapeseed oil.

Another embodiment comprises cyclosporin A, Lipo PGMS®, and Rapeseed oil.

Another embodiment comprises cyclosporin A, Liposorb S-4®, and Rapeseed oil.

Another embodiment comprises cyclosporin A, Liposorb TO-20®, and Rapeseed oil.

Another embodiment comprises cyclosporin A, Lumulse PGO®, and Rapeseed oil.

Another embodiment comprises cyclosporin A, Mackester EGDS®, and Rapeseed oil.

Another embodiment comprises cyclosporin A, Mackester EGMS®, and Rapeseed oil.

Another embodiment comprises cyclosporin A, Mackester GSTP®, and Rapeseed oil.

Another embodiment comprises cyclosporin A, Mackester Series®, and Rapeseed oil.

Another embodiment comprises cyclosporin A, Magrabar PDO-50®, and Rapeseed oil.

Another embodiment comprises cyclosporin A, Mapeg 6000 DS®, and Rapeseed oil.

Another embodiment comprises cyclosporin A, Marlowet 4702®, and Rapeseed oil.

Another embodiment comprises cyclosporin A, Monalube 305®, and Rapeseed oil.

Another embodiment comprises cyclosporin A, Monalube 310®, and Rapeseed oil.

Another embodiment comprises cyclosporin A, Monalube 315®, and Rapeseed oil.

Another embodiment comprises cyclosporin A, Monalube 320®, and Rapeseed oil.

Another embodiment comprises cyclosporin A, Monalube 325®, and Rapeseed oil.

Another embodiment comprises cyclosporin A, Monalube 330®, and Rapeseed oil.

Another embodiment comprises cyclosporin A, Naturechem PGHS®, and Rapeseed oil.

Another embodiment comprises cyclosporin A, Polycastorol PLO-840®, and Rapeseed oil.

Another embodiment comprises cyclosporin A, Polytex 10M®, and Rapeseed oil.

Another embodiment comprises cyclosporin A, Ritasynt IP®, and Rapeseed oil.

Another embodiment comprises cyclosporin A, Ross Chem PEG 600 DT®, and Rapeseed oil.

Another embodiment comprises cyclosporin A, Schercemol PGMS®, and Rapeseed oil.

Another embodiment comprises cyclosporin A, Sponto H-44C®, and Rapeseed oil.

Another embodiment comprises cyclosporin A, Tegin G®, and Rapeseed oil.

Another embodiment comprises cyclosporin A, Witbreak DOE-182®, and Rapeseed oil.

Another embodiment comprises cyclosporin A, Witbreak DOE-182®, and Rapeseed oil.

Another embodiment comprises cyclosporin A, Witbreak DRA-21®, and Rapeseed oil.

Another embodiment comprises cyclosporin A, Witbreak DRA-21®, and Rapeseed oil.

Another embodiment comprises cyclosporin A, Witbreak DRA-50®, and Rapeseed oil.

Another embodiment comprises cyclosporin A, Witbreak DRA-50®, and Rapeseed oil.

Another embodiment comprises cyclosporin A, Witconol F26-46®, and Rapeseed oil.

Another embodiment comprises cyclosporin A, Witconol H-32®, and Rapeseed oil.

Another embodiment comprises cyclosporin A, Witconol H-33®, and Rapeseed oil.

Another embodiment comprises cyclosporin A, Witconol H-35A®, and Rapeseed oil.

Another embodiment comprises cyclosporin A, Witconol RHP®, and Rapeseed oil.

Another embodiment comprises cyclosporin A, a Lanolin-based Derivative, and Rapeseed oil.

Another embodiment comprises cyclosporin A, Amerchol CAB®, and Rapeseed oil.

Another embodiment comprises cyclosporin A, Amerchol L-101®, and Rapeseed oil.

Another embodiment comprises cyclosporin A, Amerlate LFA-LO®, and Rapeseed oil.

Another embodiment comprises cyclosporin A, Amerlate P®, and Rapeseed oil.

Another embodiment comprises cyclosporin A, Barre Common Degras®, and Rapeseed oil.

Another embodiment comprises cyclosporin A, Cholesterol NF®, and Rapeseed oil.

Another embodiment comprises cyclosporin A, Crodalan AWS®, and Rapeseed oil.

Another embodiment comprises cyclosporin A, Crodalan LA®, and Rapeseed oil.

Another embodiment comprises cyclosporin A, Emery 1650®, and Rapeseed oil.

Another embodiment comprises cyclosporin A, Emery 1650®, and Rapeseed oil.

Another embodiment comprises cyclosporin A, Emery 1740®, and Rapeseed oil.

Another embodiment comprises cyclosporin A, Emery 1740®, and Rapeseed oil.

Another embodiment comprises cyclosporin A, Forlan 500®, and Rapeseed oil.

Another embodiment comprises cyclosporin A, Forlan L®, and Rapeseed oil.

Another embodiment comprises cyclosporin A, Laneto 50®, and Rapeseed oil.

Another embodiment comprises cyclosporin A, Laneto 100®, and Rapeseed oil.

Another embodiment comprises cyclosporin A, Laneto AWS®, and Rapeseed oil.

Another embodiment comprises cyclosporin A, Lanfrax 1776®, and Rapeseed oil.

Another embodiment comprises cyclosporin A, Lanfrax 1776®, and Rapeseed oil.

Another embodiment comprises cyclosporin A, Lanogel 21®, and Rapeseed oil.

Another embodiment comprises cyclosporin A, Lipolan®, and Rapeseed oil.

Another embodiment comprises cyclosporin A, Lipolan 31®, and Rapeseed oil.

Another embodiment comprises cyclosporin A, OHlan®, and Rapeseed oil.

Another embodiment comprises cyclosporin A, Polychol 5®, and Rapeseed oil.

Another embodiment comprises cyclosporin A, Polychol 15®, and Rapeseed oil.

Another embodiment comprises cyclosporin A, Ritacetyl®, and Rapeseed oil.

Another embodiment comprises cyclosporin A, Ritachol®, and Rapeseed oil.

Another embodiment comprises cyclosporin A, Ritahydrox®, and Rapeseed oil.

Another embodiment comprises cyclosporin A, Ritalafa®, and Rapeseed oil.

Another embodiment comprises cyclosporin A, Ritalan®, and Rapeseed oil.

Another embodiment comprises cyclosporin A, Ritalan AWS®, and Rapeseed oil.

Another embodiment comprises cyclosporin A, Ritalan C®, and Rapeseed oil.

Another embodiment comprises cyclosporin A, Ritawax®, and Rapeseed oil.

Another embodiment comprises cyclosporin A, Ritawax AEO®, and Rapeseed oil.

Another embodiment comprises cyclosporin A, Ritawax ALA®, and Rapeseed oil.

Another embodiment comprises cyclosporin A, Solan/Solan 50/Super Solan®, and Rapeseed oil.

Another embodiment comprises cyclosporin A, Super Hartolan/Hartolan®, and Rapeseed oil.

Another embodiment comprises cyclosporin A, Supersat AWS-4®, and Rapeseed oil.

Another embodiment comprises cyclosporin A, Supersat AWS-24®, and Rapeseed oil.

Another embodiment comprises cyclosporin A, a Lecithin or Lecithin Derivative, and Rapeseed oil.

Another embodiment comprises cyclosporin A, Alcolec®, and Rapeseed oil.

Another embodiment comprises cyclosporin A, Lecithin®, and Rapeseed oil.

Another embodiment comprises cyclosporin A, Lexin K®, and Rapeseed oil.

Another embodiment comprises cyclosporin A, Natipide®, and Rapeseed oil.

Another embodiment comprises cyclosporin A, a Lignin or Lignin Derivative, and Rapeseed oil.

Another embodiment comprises cyclosporin A, Diwatex XP 9®, and Rapeseed oil.

Another embodiment comprises cyclosporin A, Dynasperse LCD®, and Rapeseed oil.

Another embodiment comprises cyclosporin A, Indulin SAL®, and Rapeseed oil.

Another embodiment comprises cyclosporin A, Indulin W-1®, and Rapeseed oil.

Another embodiment comprises cyclosporin A, Indulin W-5®, and Rapeseed oil.

Another embodiment comprises cyclosporin A, Lignosol FTA®, and Rapeseed oil.

Another embodiment comprises cyclosporin A, Lignosol SFX-65®, and Rapeseed oil.

Another embodiment comprises cyclosporin A, Marasperse 52 CP®, and Rapeseed oil.

Another embodiment comprises cyclosporin A, Marasperse AG®, and Rapeseed oil.

Another embodiment comprises cyclosporin A, Marasperse CBOS-4®, and Rapeseed oil.

Another embodiment comprises cyclosporin A, Ufoxane 2®, and Rapeseed oil.

Another embodiment comprises cyclosporin A, a Methyl Ester, and Rapeseed oil.

Another embodiment comprises cyclosporin A, E.B. Cleaner AK®, and Rapeseed oil.

Another embodiment comprises cyclosporin A, Oleocal ME-70®, and Rapeseed oil.

Another embodiment comprises cyclosporin A, Trade Name Company®, and Rapeseed oil.

Another embodiment comprises cyclosporin A, Methyl Esters (cont'd)®, and Rapeseed oil.

Another embodiment comprises cyclosporin A, Oleocal ME-92®, and Rapeseed oil.

Another embodiment comprises cyclosporin A, Oleocal ME-112®, and Rapeseed oil.

Another embodiment comprises cyclosporin A, Oleocal ME-130®, and Rapeseed oil.

Another embodiment comprises cyclosporin A, a Monoglyceride or a Derivative thereof, and Rapeseed oil.

Another embodiment comprises cyclosporin A, Dynacet 211®, and Rapeseed oil.

Another embodiment comprises cyclosporin A, Hetsorb S-20®, and Rapeseed oil.

Another embodiment comprises cyclosporin A, Imwitor 191®, and Rapeseed oil.

Another embodiment comprises cyclosporin A, Imwitor 370®, and Rapeseed oil.

Another embodiment comprises cyclosporin A, Imwitor 375®, and Rapeseed oil.

Another embodiment comprises cyclosporin A, Imwitor 900®, and Rapeseed oil.

Another embodiment comprises cyclosporin A, Imwitor 945®, and Rapeseed oil.

Another embodiment comprises cyclosporin A, Imwitor 2020®, and Rapeseed oil.

Another embodiment comprises cyclosporin A, Kemester 5500®, and Rapeseed oil.

Another embodiment comprises cyclosporin A, Kemester 6000®, and Rapeseed oil.

Another embodiment comprises cyclosporin A, Magrabar GMC®, and Rapeseed oil.

Another embodiment comprises cyclosporin A, Magrabar GMO-CK®, and Rapeseed oil.

Another embodiment comprises cyclosporin A, Magrabar GPC-10®, and Rapeseed oil.

Another embodiment comprises cyclosporin A, Magrabar MDG-5050®, and Rapeseed oil.

Another embodiment comprises cyclosporin A, Monalube 335®, and Rapeseed oil.

Another embodiment comprises cyclosporin A, Monoglycerides Glyceryl Monestearate Archer Daniels Midland Company®, and Rapeseed oil.

Another embodiment comprises cyclosporin A, Rita GMS®, and Rapeseed oil.

Another embodiment comprises cyclosporin A, Ritamulse SCG®, and Rapeseed oil.

Another embodiment comprises cyclosporin A, Softigen 701®, and Rapeseed oil.

Another embodiment comprises cyclosporin A, Tally 100 Plus®, and Rapeseed oil.

Another embodiment comprises cyclosporin A, a Polyethylene Glycol, and Rapeseed oil.

Another embodiment comprises cyclosporin A, Emulgade PL 68/50®, and Rapeseed oil.

Another embodiment comprises cyclosporin A, Lumulse PEG®, and Rapeseed oil.

Another embodiment comprises cyclosporin A, Rhodasurf PEG-400®, and Rapeseed oil.

Another embodiment comprises cyclosporin A, Rhodasurf PEG-600®, and Rapeseed oil.

Another embodiment comprises cyclosporin A, Witconol PEG-400®, and Rapeseed oil.

Another embodiment comprises cyclosporin A, a Polymeric Sunfactant, and Rapeseed oil.

Another embodiment comprises cyclosporin A, Acritamer PNC-EG®, and Rapeseed oil.

Another embodiment comprises cyclosporin A, Ag-Rho DEP-775®, and Rapeseed oil.

Another embodiment comprises cyclosporin A, APG 325N Glycoside®, and Rapeseed oil.

Another embodiment comprises cyclosporin A, Aristoflex AVC®, and Rapeseed oil.

Another embodiment comprises cyclosporin A, Aristoflex HMB®, and Rapeseed oil.

Another embodiment comprises cyclosporin A, Burco NPS-225®, and Rapeseed oil.

Another embodiment comprises cyclosporin A, Burco NPS-816®, and Rapeseed oil.

Another embodiment comprises cyclosporin A, Chemccinate 5603®, and Rapeseed oil.

Another embodiment comprises cyclosporin A, Cosmedia Guar C-261N®, and Rapeseed oil.

Another embodiment comprises cyclosporin A, Gantrez S-95®, and Rapeseed oil.

Another embodiment comprises cyclosporin A, Glucopon 220 UP®, and Rapeseed oil.

Another embodiment comprises cyclosporin A, Glucopon 225 DK®, and Rapeseed oil.

Another embodiment comprises cyclosporin A, Glucopon 425 N®, and Rapeseed oil.

Another embodiment comprises cyclosporin A, Glucopon 600 UP®, and Rapeseed oil.

Another embodiment comprises cyclosporin A, Glucopon 625 UPF®, and Rapeseed oil.

Another embodiment comprises cyclosporin A, Pemulen 1621®, and Rapeseed oil.

Another embodiment comprises cyclosporin A, Pemulen 1622®, and Rapeseed oil.

Another embodiment comprises cyclosporin A, Pemulen TR-1®, and Rapeseed oil.

Another embodiment comprises cyclosporin A, Pemulen TR-2®, and Rapeseed oil.

Another embodiment comprises cyclosporin A, Plantacare 818®, and Rapeseed oil.

Another embodiment comprises cyclosporin A, Plantapon LGC Sorb®, and Rapeseed oil.

Another embodiment comprises cyclosporin A, Plantaren 1200N®, and Rapeseed oil.

Another embodiment comprises cyclosporin A, Plantaren 2000N®, and Rapeseed oil.

Another embodiment comprises cyclosporin A, Viscolam AT 64®, and Rapeseed oil.

Another embodiment comprises cyclosporin A, Viscolam AT 64P®, and Rapeseed oil.

Another embodiment comprises cyclosporin A, Viscolam AT 100®, and Rapeseed oil.

Another embodiment comprises cyclosporin A, Viscolam MAC 7®, and Rapeseed oil.

Another embodiment comprises cyclosporin A, Viscolam SMC 20®, and Rapeseed oil.

Another embodiment comprises cyclosporin A, Witbreak RTC-323®, and Rapeseed oil.

Another embodiment comprises cyclosporin A, WSI 3700®, and Rapeseed oil.

Another embodiment comprises cyclosporin A, a Propoxylated or Ethoxylated Fatty Acid, Alcohol, or Alkyl Phenol, and Rapeseed oil.

Another embodiment comprises cyclosporin A, Antarox AA-60®, and Rapeseed oil.

Another embodiment comprises cyclosporin A, Antarox LF-224®, and Rapeseed oil.

Another embodiment comprises cyclosporin A, Burcomul DFE-45®, and Rapeseed oil.

Another embodiment comprises cyclosporin A, Burcoterge LFE-1000®, and Rapeseed oil.

Another embodiment comprises cyclosporin A, Chemal LF-25B®, and Rapeseed oil.

Another embodiment comprises cyclosporin A, Chemal LF-40B®, and Rapeseed oil.

Another embodiment comprises cyclosporin A, Dehypon LS-36®, and Rapeseed oil.

Another embodiment comprises cyclosporin A, Dehypon LS-36®, and Rapeseed oil.

Another embodiment comprises cyclosporin A, Dehypon LS-54®, and Rapeseed oil.

Another embodiment comprises cyclosporin A, Dehypon LS-54®, and Rapeseed oil.

Another embodiment comprises cyclosporin A, Delonic 100 VLF®, and Rapeseed oil.

Another embodiment comprises cyclosporin A, Delonic LF-60 MOD®, and Rapeseed oil.

Another embodiment comprises cyclosporin A, Empiderm B®, and Rapeseed oil.

Another embodiment comprises cyclosporin A, Ethylan 1206®, and Rapeseed oil.

Another embodiment comprises cyclosporin A, Ethylan NS-500K®, and Rapeseed oil.

Another embodiment comprises cyclosporin A, Ethylan NS-500LQ®, and Rapeseed oil.

Another embodiment comprises cyclosporin A, Genapol 1392®, and Rapeseed oil.

Another embodiment comprises cyclosporin A, Genapol 2317®, and Rapeseed oil.

Another embodiment comprises cyclosporin A, Genapol 26EP710®, and Rapeseed oil.

Another embodiment comprises cyclosporin A, Genapol EP 1022®, and Rapeseed oil.

Another embodiment comprises cyclosporin A, Genapol EP 1024®, and Rapeseed oil.

Another embodiment comprises cyclosporin A, Genapol EP 6068®, and Rapeseed oil.

Another embodiment comprises cyclosporin A, Genapol NP915®, and Rapeseed oil.

Another embodiment comprises cyclosporin A, Kieralon MFB®, and Rapeseed oil.

Another embodiment comprises cyclosporin A, Lumisolve CSA-80 V®, and Rapeseed oil.

Another embodiment comprises cyclosporin A, Marlowet 5001®, and Rapeseed oil.

Another embodiment comprises cyclosporin A, Marlox FK 64®, and Rapeseed oil.

Another embodiment comprises cyclosporin A, Marlox MO 124®, and Rapeseed oil.

Another embodiment comprises cyclosporin A, Marlox S 58®, and Rapeseed oil.

Another embodiment comprises cyclosporin A, Nonatell 1003®, and Rapeseed oil.

Another embodiment comprises cyclosporin A, Nonatell 1038®, and Rapeseed oil.

Another embodiment comprises cyclosporin A, Nonatell 1052®, and Rapeseed oil.

Another embodiment comprises cyclosporin A, Nonatell 1061®, and Rapeseed oil.

Another embodiment comprises cyclosporin A, Nonatell 1075®, and Rapeseed oil.

Another embodiment comprises cyclosporin A, Nonatell 1088®, and Rapeseed oil.

Another embodiment comprises cyclosporin A, Nonatell 1123®, and Rapeseed oil.

Another embodiment comprises cyclosporin A, Nonatell 1153®, and Rapeseed oil.

Another embodiment comprises cyclosporin A, Nonatell 1161®, and Rapeseed oil.

Another embodiment comprises cyclosporin A, Nonatell 1172®, and Rapeseed oil.

Another embodiment comprises cyclosporin A, Nonatell 1181®, and Rapeseed oil.

Another embodiment comprises cyclosporin A, Norfox 36®, and Rapeseed oil.

Another embodiment comprises cyclosporin A, Procetyl AWS®, and Rapeseed oil.

Another embodiment comprises cyclosporin A, Sandoxylate SX 412®, and Rapeseed oil.

Another embodiment comprises cyclosporin A, Sandoxylate SX 418®, and Rapeseed oil.

Another embodiment comprises cyclosporin A, Surfonic JL-80X®, and Rapeseed oil.

Another embodiment comprises cyclosporin A, Suronic JL-80X-B1®, and Rapeseed oil.

Another embodiment comprises cyclosporin A, Surfonic L4-29X®, and Rapeseed oil.

Another embodiment comprises cyclosporin A, Surfonic LF®, and Rapeseed oil.

Another embodiment comprises cyclosporin A, T-Det A826®, and Rapeseed oil.

Another embodiment comprises cyclosporin A, T-Det LF-416®, and Rapeseed oil.

Another embodiment comprises cyclosporin A, Tergitol Min-Foam 1X®, and Rapeseed oil.

Another embodiment comprises cyclosporin A, Tergitol Min-Foam 2X®, and Rapeseed oil.

Another embodiment comprises cyclosporin A, Triton CF-21®, and Rapeseed oil.

Another embodiment comprises cyclosporin A, Triton CF-76®, and Rapeseed oil.

Another embodiment comprises cyclosporin A, Triton XL-80N®, and Rapeseed oil.

Another embodiment comprises cyclosporin A, Witconol NS-98®, and Rapeseed oil.

Another embodiment comprises cyclosporin A, Witconol NS-108LQ®, and Rapeseed oil.

Another embodiment comprises cyclosporin A, Witconol NS-145®, and Rapeseed oil.

Another embodiment comprises cyclosporin A, Witconol NS-179®, and Rapeseed oil.

Another embodiment comprises cyclosporin A, a Protein-based Surfactant, and Rapeseed oil.

Another embodiment comprises cyclosporin A, AminoFoam W®, and Rapeseed oil.

Another embodiment comprises cyclosporin A, Amiter LGOD-2®, and Rapeseed oil.

Another embodiment comprises cyclosporin A, Amiter LGS-2®, and Rapeseed oil.

Another embodiment comprises cyclosporin A, Amiter LGS-5®, and Rapeseed oil.

Another embodiment comprises cyclosporin A, Lamepon S®, and Rapeseed oil.

Another embodiment comprises cyclosporin A, Lamepon S®, and Rapeseed oil.

Another embodiment comprises cyclosporin A, Maypon 4C®, and Rapeseed oil.

Another embodiment comprises cyclosporin A, May-Tein C®, and Rapeseed oil.

Another embodiment comprises cyclosporin A, May-Tein CT®, and Rapeseed oil.

Another embodiment comprises cyclosporin A, May-Tein KTS®, and Rapeseed oil.

Another embodiment comprises cyclosporin A, May-Tein SY®, and Rapeseed oil.

Another embodiment comprises cyclosporin A, Plantapon S®, and Rapeseed oil.

Another embodiment comprises cyclosporin A, Proteol APL®, and Rapeseed oil.

Another embodiment comprises cyclosporin A, Proteol OAT®, and Rapeseed oil.

Another embodiment comprises cyclosporin A, Pyroter CPI-40®, and Rapeseed oil.

Another embodiment comprises cyclosporin A, Pyroter GPI-25®, and Rapeseed oil.

Another embodiment comprises cyclosporin A, Supro-Tein S®, and Rapeseed oil.

Another embodiment comprises cyclosporin A, Supro-Tein V®, and Rapeseed oil.

Another embodiment comprises cyclosporin A,® and Rapeseed oil.

Another embodiment comprises cyclosporin A, a Sarcosine Derivative®, and Rapeseed oil.

Another embodiment comprises cyclosporin A, Crodasinic LS-30®, and Rapeseed oil.

Another embodiment comprises cyclosporin A, Vanseal CS®, and Rapeseed oil.

Another embodiment comprises cyclosporin A, Vanseal LS®, and Rapeseed oil.

Another embodiment comprises cyclosporin A, Vanseal MS®, and Rapeseed oil.

Another embodiment comprises cyclosporin A, Vanseal NACS-30®, and Rapeseed oil.

Another embodiment comprises cyclosporin A, Vanseal NALS-95®, and Rapeseed oil.

Another embodiment comprises cyclosporin A, Vanseal OS®, and Rapeseed oil.

Another embodiment comprises cyclosporin A,® and Rapeseed oil.

Another embodiment comprises cyclosporin A, a Silicone-based Surfactant®, and Rapeseed oil.

Another embodiment comprises cyclosporin A, Abil-B-9950®, and Rapeseed oil.

Another embodiment comprises cyclosporin A, Abil Care 85®, and Rapeseed oil.

Another embodiment comprises cyclosporin A, Abil EM 90®, and Rapeseed oil.

Another embodiment comprises cyclosporin A, Abil EM 97®, and Rapeseed oil.

Another embodiment comprises cyclosporin A, Abil WE-09®, and Rapeseed oil.

Another embodiment comprises cyclosporin A, Dow Corning 1248 Fluid®, and Rapeseed oil.

Another embodiment comprises cyclosporin A, Dow Corning 3225C®, and Rapeseed oil.

Another embodiment comprises cyclosporin A, Dow Corning 5200®, and Rapeseed oil.

Another embodiment comprises cyclosporin A, Dow Corning Q4-3667®, and Rapeseed oil.

Another embodiment comprises cyclosporin A, Monasil PCA®, and Rapeseed oil.

Another embodiment comprises cyclosporin A, Monasil PDM®, and Rapeseed oil.

Another embodiment comprises cyclosporin A, Monasil PLN®, and Rapeseed oil.

Another embodiment comprises cyclosporin A, Polyderm PPI-SI-WS®, and Rapeseed oil.

Another embodiment comprises cyclosporin A, Troysol 380W®, and Rapeseed oil.

Another embodiment comprises cyclosporin A, Troysol S366®, and Rapeseed oil.

Another embodiment comprises cyclosporin A, a Sorbitan Derivative, and Rapeseed oil.

Another embodiment comprises cyclosporin A, Alkamuls SML®, and Rapeseed oil.

Another embodiment comprises cyclosporin A, Alkamuls SMO®, and Rapeseed oil.

Another embodiment comprises cyclosporin A, Alkamuls STO®, and Rapeseed oil.

Another embodiment comprises cyclosporin A, Arlacel 20®, and Rapeseed oil.

Another embodiment comprises cyclosporin A, Arlacel 40®, and Rapeseed oil.

Another embodiment comprises cyclosporin A, Arlacel 60®, and Rapeseed oil.

Another embodiment comprises cyclosporin A, Arlacel 80®, and Rapeseed oil.

Another embodiment comprises cyclosporin A, Arlacel C®, and Rapeseed oil.

Another embodiment comprises cyclosporin A, Armul 21®, and Rapeseed oil.

Another embodiment comprises cyclosporin A, Atlox 80®, and Rapeseed oil.

Another embodiment comprises cyclosporin A, Atlox 847®, and Rapeseed oil.

Another embodiment comprises cyclosporin A, Atlox 1045A®, and Rapeseed oil.

Another embodiment comprises cyclosporin A, Canarcel 20®, and Rapeseed oil.

Another embodiment comprises cyclosporin A, Canarcel 60®, and Rapeseed oil.

Another embodiment comprises cyclosporin A, Canarcel 80®, and Rapeseed oil.

Another embodiment comprises cyclosporin A, Canarcel TW 20®, and Rapeseed oil.

Another embodiment comprises cyclosporin A, Canarcel TW 60®, and Rapeseed oil.

Another embodiment comprises cyclosporin A, Canarcel TW 80®, and Rapeseed oil.

Another embodiment comprises cyclosporin A, Coladet BSB-P®, and Rapeseed oil.

Another embodiment comprises cyclosporin A, Customulse O-20®, and Rapeseed oil.

Another embodiment comprises cyclosporin A, Dehymuls E®, and Rapeseed oil.

Another embodiment comprises cyclosporin A, DeSotan SMO®, and Rapeseed oil.

Another embodiment comprises cyclosporin A, DeSotan SMO-20®, and Rapeseed oil.

Another embodiment comprises cyclosporin A, DeSotan SMT®, and Rapeseed oil.

Another embodiment comprises cyclosporin A, DeSotan SMT-20®, and Rapeseed oil.

Another embodiment comprises cyclosporin A, Durfax 60®, and Rapeseed oil.

Another embodiment comprises cyclosporin A, Dunax 65®, and Rapeseed oil.

Another embodiment comprises cyclosporin A, Durfax 80®, and Rapeseed oil.

Another embodiment comprises cyclosporin A, Durtan 60®, and Rapeseed oil.

Another embodiment comprises cyclosporin A, Durtan 65®, and Rapeseed oil.

Another embodiment comprises cyclosporin A, Liposorb L®, and Rapeseed oil.

Another embodiment comprises cyclosporin A, Liposorb L-10®, and Rapeseed oil.

Another embodiment comprises cyclosporin A, Liposorb L-20®, and Rapeseed oil.

Another embodiment comprises cyclosporin A, Liposorb O®, and Rapeseed oil.

Another embodiment comprises cyclosporin A, Liposorb O-20®, and Rapeseed oil.

Another embodiment comprises cyclosporin A, Liposorb P®, and Rapeseed oil.

Another embodiment comprises cyclosporin A, Liposorb P-20®, and Rapeseed oil.

Another embodiment comprises cyclosporin A, Liposorb S®, and Rapeseed oil.

Another embodiment comprises cyclosporin A, Liposorb S-20®, and Rapeseed oil.

Another embodiment comprises cyclosporin A, Liposorb SQO®, and Rapeseed oil.

Another embodiment comprises cyclosporin A, Liposorb TO®, and Rapeseed oil.

Another embodiment comprises cyclosporin A, Liposorb TS®, and Rapeseed oil.

Another embodiment comprises cyclosporin A, Liposorb TS-20®, and Rapeseed oil.

Another embodiment comprises cyclosporin A, Lumisorb PS®, and Rapeseed oil.

Another embodiment comprises cyclosporin A, Lumisorb SMO (T)®, and Rapeseed oil.

Another embodiment comprises cyclosporin A, Lumisorb SMS K®, and Rapeseed oil.

Another embodiment comprises cyclosporin A, Lumisorb SSO®, and Rapeseed oil.

Another embodiment comprises cyclosporin A, Lumisorb STS K®, and Rapeseed oil.

Another embodiment comprises cyclosporin A, Lumisorb STT®, and Rapeseed oil.

Another embodiment comprises cyclosporin A, Magrabar SMO®, and Rapeseed oil.

Another embodiment comprises cyclosporin A, Magrabar SMO-VEG®, and Rapeseed oil.

Another embodiment comprises cyclosporin A, Magrabar SMT®, and Rapeseed oil.

Another embodiment comprises cyclosporin A, Magrabar STO®, and Rapeseed oil.

Another embodiment comprises cyclosporin A, Miracare BC-27®, and Rapeseed oil.

Another embodiment comprises cyclosporin A, Ritabate 20®, and Rapeseed oil.

Another embodiment comprises cyclosporin A, Ritabate 40®, and Rapeseed oil.

Another embodiment comprises cyclosporin A, Ritabate 60®, and Rapeseed oil.

Another embodiment comprises cyclosporin A, Ritabate 80®, and Rapeseed oil.

Another embodiment comprises cyclosporin A, T-Maz®, and Rapeseed oil.

Another embodiment comprises cyclosporin A, Tego SML®, and Rapeseed oil.

Another embodiment comprises cyclosporin A, Tego SML 20®, and Rapeseed oil.

Another embodiment comprises cyclosporin A, Tego SMO 80 V®, and Rapeseed oil.

Another embodiment comprises cyclosporin A, Tego SMO V®, and Rapeseed oil.

Another embodiment comprises cyclosporin A, Tego SMS®, and Rapeseed oil.

Another embodiment comprises cyclosporin A, Tego STO V®, and Rapeseed oil.

Another embodiment comprises cyclosporin A, Tween 21®, and Rapeseed oil.

Another embodiment comprises cyclosporin A, Tween 40®, and Rapeseed oil.

Another embodiment comprises cyclosporin A, Tween 60®, and Rapeseed oil.

Another embodiment comprises cyclosporin A, Tween 60 K®, and Rapeseed oil.

Another embodiment comprises cyclosporin A, Tween 61®, and Rapeseed oil.

Another embodiment comprises cyclosporin A, Tween 65®, and Rapeseed oil.

Another embodiment comprises cyclosporin A, Tween 80®, and Rapeseed oil.

Another embodiment comprises cyclosporin A, Tween 80 K®, and Rapeseed oil.

Another embodiment comprises cyclosporin A, Tween 81®, and Rapeseed oil.

Another embodiment comprises cyclosporin A, Tween 85®, and Rapeseed oil.

Another embodiment comprises cyclosporin A, a Sucrose or Glucose Ester, or Derivative thereof, and Rapeseed oil.

Another embodiment comprises cyclosporin A, DeSulf GOS-P-60WCG®, and Rapeseed oil.

Another embodiment comprises cyclosporin A, Glucam E-20 Distearate®, and Rapeseed oil.

Another embodiment comprises cyclosporin A, Glucamate DOE-120®, and Rapeseed oil.

Another embodiment comprises cyclosporin A, Glucamate SSE-20®, and Rapeseed oil.

Another embodiment comprises cyclosporin A, Glucate DO®, and Rapeseed oil.

Another embodiment comprises cyclosporin A, Glucate SS®, and Rapeseed oil.

Another embodiment comprises cyclosporin A, Glucopon 425 UP®, and Rapeseed oil.

Another embodiment comprises cyclosporin A, Isolan IS®, and Rapeseed oil.

Another embodiment comprises cyclosporin A, Mazon 40®, and Rapeseed oil.

Another embodiment comprises cyclosporin A, Montanov 82®, and Rapeseed oil.

Another embodiment comprises cyclosporin A, Montanov 202®, and Rapeseed oil.

Another embodiment comprises cyclosporin A, Montanov S®, and Rapeseed oil.

Another embodiment comprises cyclosporin A, Rheozan®, and Rapeseed oil.

Another embodiment comprises cyclosporin A, Simulsol AS 48®, and Rapeseed oil.

Another embodiment comprises cyclosporin A, Simulsol SL 4®, and Rapeseed oil.

Another embodiment comprises cyclosporin A, Simulsol SL 10®, and Rapeseed oil.

Another embodiment comprises cyclosporin A, Simulsol SL 11 W®, and Rapeseed oil.

Another embodiment comprises cyclosporin A, Simulsol SL 55®, and Rapeseed oil.

Another embodiment comprises cyclosporin A, Suga Nate 100 and 160®, and Rapeseed oil.

Another embodiment comprises cyclosporin A, Tego Care 450®, and Rapeseed oil.

Another embodiment comprises cyclosporin A, Tego Care CG 90®, and Rapeseed oil.

Another embodiment comprises cyclosporin A, Tego Care PS®, and Rapeseed oil.

Another embodiment comprises cyclosporin A, Tegosoft PSE 141 G®, and Rapeseed oil.

Another embodiment comprises cyclosporin A, Tegotens G 826®, and Rapeseed oil.

Another embodiment comprises cyclosporin A, Triton BG-10 (70%)®, and Rapeseed oil.

Another embodiment comprises cyclosporin A, Triton CG-110 (60%)®, and Rapeseed oil.

Another embodiment comprises cyclosporin A, Wickenol 545®, and Rapeseed oil.

Another embodiment comprises cyclosporin A, an alcohol, and Soybean oil.

Another embodiment comprises cyclosporin A, Diglycerol®, and Soybean oil.

Another embodiment comprises cyclosporin A, Hetoxide GT-80®, and Soybean oil.

Another embodiment comprises cyclosporin A, Lexemul BEO®, and Soybean oil.

Another embodiment comprises cyclosporin A, Polyglycerol-3®, and Soybean oil.

Another embodiment comprises cyclosporin A, Redicote E Series®, and Soybean oil.

Another embodiment comprises cyclosporin A, Simulsol OX 1005L®, and Soybean oil.

Another embodiment comprises cyclosporin A, Stanfax 567®, and Soybean oil.

Another embodiment comprises cyclosporin A, TA-1618®, and Soybean oil.

Another embodiment comprises cyclosporin A, Witconol H-31A®, and Soybean oil.

Another embodiment comprises cyclosporin A, an amine oxide, and Soybean oil.

Another embodiment comprises cyclosporin A, AO-405®, and Soybean oil.

Another embodiment comprises cyclosporin A, AO-455®, and Soybean oil.

Another embodiment comprises cyclosporin A, AO 728 Special®, and Soybean oil.

Another embodiment comprises cyclosporin A, Barlox 12®, and Soybean oil.

Another embodiment comprises cyclosporin A, Barlox 14®, and Soybean oil.

Another embodiment comprises cyclosporin A, Burcoxide Lo®, and Soybean oil.

Another embodiment comprises cyclosporin A, Caloxamine LO®, and Soybean oil.

Another embodiment comprises cyclosporin A, Chemoxide CAW®, and Soybean oil.

Another embodiment comprises cyclosporin A, Chemoxide LM-30®, and Soybean oil.

Another embodiment comprises cyclosporin A, Chemoxide LO®, and Soybean oil.

Another embodiment comprises cyclosporin A, Chemoxide MO®, and Soybean oil.

Another embodiment comprises cyclosporin A, Colalux CAO-35®, and Soybean oil.

Another embodiment comprises cyclosporin A, Colalux LO®, and Soybean oil.

Another embodiment comprises cyclosporin A, DeMox CAPO®, and Soybean oil.

Another embodiment comprises cyclosporin A, DeMox CSG-30®, and Soybean oil.

Another embodiment comprises cyclosporin A, DeMox LAO®, and Soybean oil.

Another embodiment comprises cyclosporin A, Emcol L®, and Soybean oil.

Another embodiment comprises cyclosporin A, Empigen OB®, and Soybean oil.

Another embodiment comprises cyclosporin A, Empigen OS/A®, and Soybean oil.

Another embodiment comprises cyclosporin A, Foamox CDO®, and Soybean oil.

Another embodiment comprises cyclosporin A, Foamox DMM®, and Soybean oil.

Another embodiment comprises cyclosporin A, Foamox DMS®, and Soybean oil.

Another embodiment comprises cyclosporin A, Genaminox KC®, and Soybean oil.

Another embodiment comprises cyclosporin A, Genaminox LA®, and Soybean oil.

Another embodiment comprises cyclosporin A, Hartotoam SAO®, and Soybean oil.

Another embodiment comprises cyclosporin A, Hartox DMCD®, and Soybean oil.

Another embodiment comprises cyclosporin A, Lipowax DAT®, and Soybean oil.

Another embodiment comprises cyclosporin A, Lipowax PB Pastilles®, and Soybean oil.

Another embodiment comprises cyclosporin A, Mackamine C8®, and Soybean oil.

Another embodiment comprises cyclosporin A, Mackamine C10®, and Soybean oil.

Another embodiment comprises cyclosporin A, Mackamine C14®, and Soybean oil.

Another embodiment comprises cyclosporin A, Mackamine CAO®, and Soybean oil.

Another embodiment comprises cyclosporin A, Mackamine CO®, and Soybean oil.

Another embodiment comprises cyclosporin A, Mackamine LO®, and Soybean oil.

Another embodiment comprises cyclosporin A, Mackamine O2®, and Soybean oil.

Another embodiment comprises cyclosporin A, Mackamine SAO®, and Soybean oil.

Another embodiment comprises cyclosporin A, Mackamine SO®, and Soybean oil.

Another embodiment comprises cyclosporin A, Mazox KCAO®, and Soybean oil.

Another embodiment comprises cyclosporin A, Monalac MO®, and Soybean oil.

Another embodiment comprises cyclosporin A, Norfox LDA®, and Soybean oil.

Another embodiment comprises cyclosporin A, Rhodamox LO®, and Soybean oil.

Another embodiment comprises cyclosporin A, Schercamox C-AA®, and Soybean oil.

Another embodiment comprises cyclosporin A, Schercamox DMA®, and Soybean oil.

Another embodiment comprises cyclosporin A, Schercamox DML®, and Soybean oil.

Another embodiment comprises cyclosporin A, Schercamox DMM®, and Soybean oil.

Another embodiment comprises cyclosporin A, Schercamox DMS®, and Soybean oil.

Another embodiment comprises cyclosporin A, Tegotens DO®, and Soybean oil.

Another embodiment comprises cyclosporin A, Tomah AO-14-2®, and Soybean oil.

Another embodiment comprises cyclosporin A, Triaminox CDO®, and Soybean oil.

Another embodiment comprises cyclosporin A, a block polymer, and Soybean oil.

Another embodiment comprises cyclosporin A, AL 2070®, and Soybean oil.

Another embodiment comprises cyclosporin A, Antarox 17-R-2®, and Soybean oil.

Another embodiment comprises cyclosporin A, Antarox 25-R-2®, and Soybean oil.

Another embodiment comprises cyclosporin A, Antarox 31-R-1®, and Soybean oil.

Another embodiment comprises cyclosporin A, Antarox P-84®, and Soybean oil.

Another embodiment comprises cyclosporin A, Antarox P-104/H®, and Soybean oil.

Another embodiment comprises cyclosporin A, Arnox BP-Series®, and Soybean oil.

Another embodiment comprises cyclosporin A, Chemonic 435®, and Soybean oil.

Another embodiment comprises cyclosporin A, Chemonic D-25®, and Soybean oil.

Another embodiment comprises cyclosporin A, Chemonic PL Seriese®, and Soybean oil.

Another embodiment comprises cyclosporin A, Ethox L-121®, and Soybean oil.

Another embodiment comprises cyclosporin A, Ethox L-122®, and Soybean oil.

Another embodiment comprises cyclosporin A, Genapol PF-10®, and Soybean oil.

Another embodiment comprises cyclosporin A, Genapol PF-20®, and Soybean oil.

Another embodiment comprises cyclosporin A, Genapol PF-40A®, and Soybean oil.

Another embodiment comprises cyclosporin A, Norfox 2-LF®, and Soybean oil.

Another embodiment comprises cyclosporin A, Simulsol NW 342®, and Soybean oil.

Another embodiment comprises cyclosporin A, T-Det BP-1®, and Soybean oil.

Another embodiment comprises cyclosporin A, T-Det XD®, and Soybean oil.

Another embodiment comprises cyclosporin A, T-Det XH®, and Soybean oil.

Another embodiment comprises cyclosporin A, Triton CF-32®, and Soybean oil.

Another embodiment comprises cyclosporin A, Witconol 171®, and Soybean oil.

Another embodiment comprises cyclosporin A, Witconol 324®, and Soybean oil.

Another embodiment comprises cyclosporin A, Witconol 324®, and Soybean oil.

Another embodiment comprises cyclosporin A, Witconol PD-2000®, and Soybean oil.

Another embodiment comprises cyclosporin A, a Carboxylated Alcohol or Alkylphenol Ethoxylate, and Soybean oil.

Another embodiment comprises cyclosporin A, Emcol CN-6®, and Soybean oil.

Another embodiment comprises cyclosporin A, Ethcarb®, and Soybean oil.

Another embodiment comprises cyclosporin A, Gemtex WNT-Conc®, and Soybean oil.

Another embodiment comprises cyclosporin A, Incrodet TD7-C®, and Soybean oil.

Another embodiment comprises cyclosporin A, Marlinat CM 105/80®, and Soybean oil.

Another embodiment comprises cyclosporin A, Marlowet 1072®, and Soybean oil.

Another embodiment comprises cyclosporin A, Marlowet 4530®, and Soybean oil.

Another embodiment comprises cyclosporin A, Marlowet 4530 LF®, and Soybean oil.

Another embodiment comprises cyclosporin A, Marlowet 4534®, and Soybean oil.

Another embodiment comprises cyclosporin A, Marlowet 4538®, and Soybean oil.

Another embodiment comprises cyclosporin A, Marlowet 4539®, and Soybean oil.

Another embodiment comprises cyclosporin A, Marlowet 4539 LF®, and Soybean oil.

Another embodiment comprises cyclosporin A, Marlowet 4541®, and Soybean oil.

Another embodiment comprises cyclosporin A, Miranate LEC-80®, and Soybean oil.

Another embodiment comprises cyclosporin A, Sandopan B®, and Soybean oil.

Another embodiment comprises cyclosporin A, Sandopan B Modified®, and Soybean oil.

Another embodiment comprises cyclosporin A, Sandopan LS-24 Gel®, and Soybean oil.

Another embodiment comprises cyclosporin A, Surfine T-A®, and Soybean oil.

Another embodiment comprises cyclosporin A, a Carboxylic Acid/Fatty Acid, and Soybean oil.

Another embodiment comprises cyclosporin A, Colaterge RAM®, and Soybean oil.

Another embodiment comprises cyclosporin A, Colatrope INC®, and Soybean oil.

Another embodiment comprises cyclosporin A, Crodacid B®, and Soybean oil.

Another embodiment comprises cyclosporin A, DeTrope CA-100®, and Soybean oil.

Another embodiment comprises cyclosporin A, Latol MTO®, and Soybean oil.

Another embodiment comprises cyclosporin A, Lumulse CC-33 K®, and Soybean oil.

Another embodiment comprises cyclosporin A, Mulls 2218®, and Soybean oil.

Another embodiment comprises cyclosporin A, OL-600®, and Soybean oil.

Another embodiment comprises cyclosporin A, OL-800®, and Soybean oil.

Another embodiment comprises cyclosporin A, R-910®, and Soybean oil.

Another embodiment comprises cyclosporin A, S-210®, and Soybean oil.

Another embodiment comprises cyclosporin A, Sandopan DTC Acid®, and Soybean oil.

Another embodiment comprises cyclosporin A, Sandopan LS 24 N®, and Soybean oil.

Another embodiment comprises cyclosporin A, Sandopan MA-18®, and Soybean oil.

Another embodiment comprises cyclosporin A, an Ethoxylated Alcohol, and Soybean oil.

Another embodiment comprises cyclosporin A, Adsee 799®, and Soybean oil.

Another embodiment comprises cyclosporin A, Adsee 799®, and Soybean oil.

Another embodiment comprises cyclosporin A, Alfonic 610-3.5®, and Soybean oil.

Another embodiment comprises cyclosporin A, Alfonic 810-2®, and Soybean oil.

Another embodiment comprises cyclosporin A, Alfonic 810-6®, and Soybean oil.

Another embodiment comprises cyclosporin A, Alfonic 1012-3®, and Soybean oil.

Another embodiment comprises cyclosporin A, Alfonic 1012-5®, and Soybean oil.

Another embodiment comprises cyclosporin A, Alfonic 1216CO-1.5®, and Soybean oil.

Another embodiment comprises cyclosporin A, Alfonic 1216CO-7®, and Soybean oil.

Another embodiment comprises cyclosporin A, Alfonic 1412-3®, and Soybean oil.

Another embodiment comprises cyclosporin A, Alfonic 1412-7®, and Soybean oil.

Another embodiment comprises cyclosporin A, Arlasolve 200®, and Soybean oil.

Another embodiment comprises cyclosporin A, Arlasolve 200 Liquid®, and Soybean oil.

Another embodiment comprises cyclosporin A, Armix 180-C®, and Soybean oil.

Another embodiment comprises cyclosporin A, Armix 183®, and Soybean oil.

Another embodiment comprises cyclosporin A, Armul 2404®, and Soybean oil.

Another embodiment comprises cyclosporin A, Armul 2404®, and Soybean oil.

Another embodiment comprises cyclosporin A, Atlas EMJ-C®, and Soybean oil.

Another embodiment comprises cyclosporin A, Atlas G-2109®, and Soybean oil.

Another embodiment comprises cyclosporin A, Atlas G-3886®, and Soybean oil.

Another embodiment comprises cyclosporin A, Atlas G-3890®, and Soybean oil.

Another embodiment comprises cyclosporin A, Bio Soft E-200®, and Soybean oil.

Another embodiment comprises cyclosporin A, Bio Soft E-300®, and Soybean oil.

Another embodiment comprises cyclosporin A, Bio Soft E-400®, and Soybean oil.

Another embodiment comprises cyclosporin A, Bio Soft EN 600®, and Soybean oil.

Another embodiment comprises cyclosporin A, Bio Soft TD-400®, and Soybean oil.

Another embodiment comprises cyclosporin A, Bio Soft TD-630®, and Soybean oil.

Another embodiment comprises cyclosporin A, Brij 30®, and Soybean oil.

Another embodiment comprises cyclosporin A, Brij 52®, and Soybean oil.

Another embodiment comprises cyclosporin A, Brij 56®, and Soybean oil.

Another embodiment comprises cyclosporin A, Brij 58®, and Soybean oil.

Another embodiment comprises cyclosporin A, Brij 72®, and Soybean oil.

Another embodiment comprises cyclosporin A, Brij 76®, and Soybean oil.

Another embodiment comprises cyclosporin A, Brij 78®, and Soybean oil.

Another embodiment comprises cyclosporin A, Brij 93®, and Soybean oil.

Another embodiment comprises cyclosporin A, Brij 97®, and Soybean oil.

Another embodiment comprises cyclosporin A, Brij 98®, and Soybean oil.

Another embodiment comprises cyclosporin A, Brij 700®, and Soybean oil.

Another embodiment comprises cyclosporin A, Brij 700 S®, and Soybean oil.

Another embodiment comprises cyclosporin A, Brij 721®, and Soybean oil.

Another embodiment comprises cyclosporin A, Brij 721 S®, and Soybean oil.

Another embodiment comprises cyclosporin A, Burcoterge CDG®, and Soybean oil.

Another embodiment comprises cyclosporin A, Canasol AT 600®, and Soybean oil.

Another embodiment comprises cyclosporin A, Canasol AT 800®, and Soybean oil.

Another embodiment comprises cyclosporin A, Canasol AT 1200®, and Soybean oil.

Another embodiment comprises cyclosporin A, Canasol BJ 35®, and Soybean oil.

Another embodiment comprises cyclosporin A, Canasol BJ 36®, and Soybean oil.

Another embodiment comprises cyclosporin A, Canasol BJ 52®, and Soybean oil.

Another embodiment comprises cyclosporin A, Canasol BJ 58®, and Soybean oil.

Another embodiment comprises cyclosporin A, Canasol BJ 72®, and Soybean oil.

Another embodiment comprises cyclosporin A, Canasol BJ 78®, and Soybean oil.

Another embodiment comprises cyclosporin A, Canasol BJ 98®, and Soybean oil.

Another embodiment comprises cyclosporin A, Canasol BJ 307®, and Soybean oil.

Another embodiment comprises cyclosporin A, Cenak 1400®, and Soybean oil.

Another embodiment comprises cyclosporin A, Cetomacrogol 1000 BP®, and Soybean oil.

Another embodiment comprises cyclosporin A, Chemonic C-2®, and Soybean oil.

Another embodiment comprises cyclosporin A, Chemonic C-10®, and Soybean oil.

Another embodiment comprises cyclosporin A, Chemonic C-20®, and Soybean oil.

Another embodiment comprises cyclosporin A, Chemonic CT-12®, and Soybean oil.

Another embodiment comprises cyclosporin A, Chemonic CT-20®, and Soybean oil.

Another embodiment comprises cyclosporin A, Chemonic CT-30®, and Soybean oil.

Another embodiment comprises cyclosporin A, Chemonic CT-55®, and Soybean oil.

Another embodiment comprises cyclosporin A, Chemonic G-7®, and Soybean oil.

Another embodiment comprises cyclosporin A, Chemonic G-26®, and Soybean oil.

Another embodiment comprises cyclosporin A, Chemonic L-4®, and Soybean oil.

Another embodiment comprises cyclosporin A, Chemonic L-7®, and Soybean oil.

Another embodiment comprises cyclosporin A, Chemonic L-12®, and Soybean oil.

Another embodiment comprises cyclosporin A, Chemonic L-23®, and Soybean oil.

Another embodiment comprises cyclosporin A, Chemonic O-2®, and Soybean oil.

Another embodiment comprises cyclosporin A, Chemonic O-5®, and Soybean oil.

Another embodiment comprises cyclosporin A, Chemonic O-10®, and Soybean oil.

Another embodiment comprises cyclosporin A, Chemonic O-20®, and Soybean oil.

Another embodiment comprises cyclosporin A, Chemonic S-2®, and Soybean oil.

Another embodiment comprises cyclosporin A, Chemonic S-10®, and Soybean oil.

Another embodiment comprises cyclosporin A, Chemonic S-20®, and Soybean oil.

Another embodiment comprises cyclosporin A, Colamulse FE®, and Soybean oil.

Another embodiment comprises cyclosporin A, Cremophor A 20®, and Soybean oil.

Another embodiment comprises cyclosporin A, Cremophor SA 2®, and Soybean oil.

Another embodiment comprises cyclosporin A, Dehydol 100®, and Soybean oil.

Another embodiment comprises cyclosporin A, Dehydol O-4®, and Soybean oil.

Another embodiment comprises cyclosporin A, Delonic C-18®, and Soybean oil.

Another embodiment comprises cyclosporin A, DeSonic 6T®, and Soybean oil.

Another embodiment comprises cyclosporin A, DeSonic 9D®, and Soybean oil.

Another embodiment comprises cyclosporin A, DeSonic 9T®, and Soybean oil.

Another embodiment comprises cyclosporin A, DeSonic 12D®, and Soybean oil.

Another embodiment comprises cyclosporin A, DeSonic 12T®, and Soybean oil.

Another embodiment comprises cyclosporin A, DeSonic 15T®, and Soybean oil.

Another embodiment comprises cyclosporin A, DeSonic TDA-9®, and Soybean oil.

Another embodiment comprises cyclosporin A, DeThox GLG-7®, and Soybean oil.

Another embodiment comprises cyclosporin A, DeThox GLG-26®, and Soybean oil.

Another embodiment comprises cyclosporin A, DeThox LA-4®, and Soybean oil.

Another embodiment comprises cyclosporin A, DeThox LA-23®, and Soybean oil.

Another embodiment comprises cyclosporin A, DeThox SA-80®, and Soybean oil.

Another embodiment comprises cyclosporin A, Disponil O5®, and Soybean oil.

Another embodiment comprises cyclosporin A, Eccoterge EO-41B®, and Soybean oil.

Another embodiment comprises cyclosporin A, Empilan KA2.5/90®, and Soybean oil.

Another embodiment comprises cyclosporin A, Empilan KA5/90®, and Soybean oil.

Another embodiment comprises cyclosporin A, Empilan KM-20®, and Soybean oil.

Another embodiment comprises cyclosporin A, Empilan KM-50®, and Soybean oil.

Another embodiment comprises cyclosporin A, Empilan L-23®, and Soybean oil.

Another embodiment comprises cyclosporin A, Ethylan 25-3®, and Soybean oil.

Another embodiment comprises cyclosporin A, Ethylan 1204®, and Soybean oil.

Another embodiment comprises cyclosporin A, Ethylan DA-4®, and Soybean oil.

Another embodiment comprises cyclosporin A, Ethylan LA-230®, and Soybean oil.

Another embodiment comprises cyclosporin A, Ethylan SN®, and Soybean oil.

Another embodiment comprises cyclosporin A, Ethylan TD-60®, and Soybean oil.

Another embodiment comprises cyclosporin A, Ethylan TD-100®, and Soybean oil.

Another embodiment comprises cyclosporin A, Ethylan TD-1407®, and Soybean oil.

Another embodiment comprises cyclosporin A, Eumulgin B1®, and Soybean oil.

Another embodiment comprises cyclosporin A, Eumulgin B2®, and Soybean oil.

Another embodiment comprises cyclosporin A, Eumulgin B3®, and Soybean oil.

Another embodiment comprises cyclosporin A, Eumulgin O-10®, and Soybean oil.

Another embodiment comprises cyclosporin A, Flo Mo 80/20®, and Soybean oil.

Another embodiment comprises cyclosporin A, Flo Mo Low Foam®, and Soybean oil.

Another embodiment comprises cyclosporin A, Forlan C-24®, and Soybean oil.

Another embodiment comprises cyclosporin A, Genapol 1454®, and Soybean oil.

Another embodiment comprises cyclosporin A, Genapol BA-020®, and Soybean oil.

Another embodiment comprises cyclosporin A, Genapol BA-040®, and Soybean oil.

Another embodiment comprises cyclosporin A, Genapol C-100®, and Soybean oil.

Another embodiment comprises cyclosporin A, Genapbl DA 060®, and Soybean oil.

Another embodiment comprises cyclosporin A, Genapol HS 020®, and Soybean oil.

Another embodiment comprises cyclosporin A, Genapol HS 200®, and Soybean oil.

Another embodiment comprises cyclosporin A, Genapol ID-040®, and Soybean oil.

Another embodiment comprises cyclosporin A, Genapol ID-060®, and Soybean oil.

Another embodiment comprises cyclosporin A, Genapol ID-090®, and Soybean oil.

Another embodiment comprises cyclosporin A, Genapol LA 010®, and Soybean oil.

Another embodiment comprises cyclosporin A, Genapol LA 020®, and Soybean oil.

Another embodiment comprises cyclosporin A, Genapol LA 030®, and Soybean oil.

Another embodiment comprises cyclosporin A, Genapol LA 040®, and Soybean oil.

Another embodiment comprises cyclosporin A, Genapol LA 050®, and Soybean oil.

Another embodiment comprises cyclosporin A, Genapol LA 060®, and Soybean oil.

Another embodiment comprises cyclosporin A, Genapol LA 070®, and Soybean oil.

Another embodiment comprises cyclosporin A, Genapol LA 070S®, and Soybean oil.

Another embodiment comprises cyclosporin A, Genapol LA 230®, and Soybean oil.

Another embodiment comprises cyclosporin A, Genapol O 020®, and Soybean oil.

Another embodiment comprises cyclosporin A, Genapol O 050®, and Soybean oil.

Another embodiment comprises cyclosporin A, Genapol O 100®, and Soybean oil.

Another embodiment comprises cyclosporin A, Genapol O 200®, and Soybean oil.

Another embodiment comprises cyclosporin A, Genapol SA 030®, and Soybean oil.

Another embodiment comprises cyclosporin A, Genapol SA 120®, and Soybean oil.

Another embodiment comprises cyclosporin A, Genapol T-020®, and Soybean oil.

Another embodiment comprises cyclosporin A, Genapol UD-030®, and Soybean oil.

Another embodiment comprises cyclosporin A, Genapol UD-050®, and Soybean oil.

Another embodiment comprises cyclosporin A, Genapol UD-070®, and Soybean oil.

Another embodiment comprises cyclosporin A, Genapol UD-079®, and Soybean oil.

Another embodiment comprises cyclosporin A, Genapol UD-080®, and Soybean oil.

Another embodiment comprises cyclosporin A, Genapol UD-110®, and Soybean oil.

Another embodiment comprises cyclosporin A, Genapol X 030®, and Soybean oil.

Another embodiment comprises cyclosporin A, Genapol X 050®, and Soybean oil.

Another embodiment comprises cyclosporin A, Genapol X 060®, and Soybean oil.

Another embodiment comprises cyclosporin A, Genapol X 070®, and Soybean oil.

Another embodiment comprises cyclosporin A, Genapol X 080®, and Soybean oil.

Another embodiment comprises cyclosporin A, Genapol X 100®, and Soybean oil.

Another embodiment comprises cyclosporin A, Genapol X 159®, and Soybean oil.

Another embodiment comprises cyclosporin A, Generol 122 E5®, and Soybean oil.

Another embodiment comprises cyclosporin A, Generol 122 E25®, and Soybean oil.

Another embodiment comprises cyclosporin A, Hostacerin T-3®, and Soybean oil.

Another embodiment comprises cyclosporin A, Iconol LF 110®, and Soybean oil.

Another embodiment comprises cyclosporin A, Incropol CS-20®, and Soybean oil.

Another embodiment comprises cyclosporin A, Lexemul CS-20®, and Soybean oil.

Another embodiment comprises cyclosporin A, Liponic EG-1®, and Soybean oil.

Another embodiment comprises cyclosporin A, Lipowax D®, and Soybean oil.

Another embodiment comprises cyclosporin A, Lipowax G®, and Soybean oil.

Another embodiment comprises cyclosporin A, Lipowax NI®, and Soybean oil.

Another embodiment comprises cyclosporin A, Lipowax P®, and Soybean oil.

Another embodiment comprises cyclosporin A, Lipowax P-31®, and Soybean oil.

Another embodiment comprises cyclosporin A, Lipowax PR®, and Soybean oil.

Another embodiment comprises cyclosporin A, Lumulse CS-20®, and Soybean oil.

Another embodiment comprises cyclosporin A, Macol CSA-20®, and Soybean oil.

Another embodiment comprises cyclosporin A, Marlox B 24/50®, and Soybean oil.

Another embodiment comprises cyclosporin A, Mazawet 77®, and Soybean oil.

Another embodiment comprises cyclosporin A, Nortox 1713®, and Soybean oil.

Another embodiment comprises cyclosporin A, Norfox 2579®, and Soybean oil.

Another embodiment comprises cyclosporin A, Norfox Lo Foam®, and Soybean oil.

Another embodiment comprises cyclosporin A, Promulgen D®, and Soybean oil.

Another embodiment comprises cyclosporin A, Promulgen G®, and Soybean oil.

Another embodiment comprises cyclosporin A, Renex 30®, and Soybean oil.

Another embodiment comprises cyclosporin A, Renex 36®, and Soybean oil.

Another embodiment comprises cyclosporin A, Rhodasurf A 24®, and Soybean oil.

Another embodiment comprises cyclosporin A, Rhodasurf AAE-10®, and Soybean oil.

Another embodiment comprises cyclosporin A, Rhodasurf BEH-25®, and Soybean oil.

Another embodiment comprises cyclosporin A, Rhodasurf BEH-40®, and Soybean oil.

Another embodiment comprises cyclosporin A, Rhodasurf DA 530®, and Soybean oil.

Another embodiment comprises cyclosporin A, Rhodasurf DA 630®, and Soybean oil.

Another embodiment comprises cyclosporin A, Rhodasurf DA 639®, and Soybean oil.

Another embodiment comprises cyclosporin A, Rhodasurf LAN-23®, and Soybean oil.

Another embodiment comprises cyclosporin A, Rhodasurf ON-870®, and Soybean oil.

Another embodiment comprises cyclosporin A, Rhodasurf ON-877®, and Soybean oil.

Another embodiment comprises cyclosporin A, Rhodasurf TB-970 FLK®, and Soybean oil.

Another embodiment comprises cyclosporin A, Ritacet-20®, and Soybean oil.

Another embodiment comprises cyclosporin A, Ritachol 1000®, and Soybean oil.

Another embodiment comprises cyclosporin A, Ritachol 2000®, and Soybean oil.

Another embodiment comprises cyclosporin A, Ritachol 5000®, and Soybean oil.

Another embodiment comprises cyclosporin A, Ritox 35®, and Soybean oil.

Another embodiment comprises cyclosporin A, Surfonic DA-4®, and Soybean oil.

Another embodiment comprises cyclosporin A, Surfonic DA-6®, and Soybean oil.

Another embodiment comprises cyclosporin A, Surfonic L46-7®, and Soybean oil.

Another embodiment comprises cyclosporin A, Surfonic POA®, and Soybean oil.

Another embodiment comprises cyclosporin A, Synthrapol KB®, and Soybean oil.

Another embodiment comprises cyclosporin A, Teginacids®, and Soybean oil.

Another embodiment comprises cyclosporin A, Teginacid C®, and Soybean oil.

Another embodiment comprises cyclosporin A, Tegotens EC 11®, and Soybean oil.

Another embodiment comprises cyclosporin A, Tinegal NA®, and Soybean oil.

Another embodiment comprises cyclosporin A, Tomadol 400®, and Soybean oil.

Another embodiment comprises cyclosporin A, Tomadol 600®, and Soybean oil.

Another embodiment comprises cyclosporin A, Tomadol 900®, and Soybean oil.

Another embodiment comprises cyclosporin A, Uniperol O®, and Soybean oil.

Another embodiment comprises cyclosporin A, Witconol SN Series®, and Soybean oil.

Another embodiment comprises cyclosporin A, an Ethoxylated Alkylphenol, and Soybean oil.

Another embodiment comprises cyclosporin A, Antarox LF-222®, and Soybean oil.

Another embodiment comprises cyclosporin A, Atlox 775®, and Soybean oil.

Another embodiment comprises cyclosporin A, Caloxylate N-9®, and Soybean oil.

Another embodiment comprises cyclosporin A, Canasol NF-1000®, and Soybean oil.

Another embodiment comprises cyclosporin A, Canasol NF-3000®, and Soybean oil.

Another embodiment comprises cyclosporin A, Canasol NF-3070®, and Soybean oil.

Another embodiment comprises cyclosporin A, Canasol OF 1670®, and Soybean oil.

Another embodiment comprises cyclosporin A, Canasol OF 2570®, and Soybean oil.

Another embodiment comprises cyclosporin A, Canasol OF 4070®, and Soybean oil.

Another embodiment comprises cyclosporin A, Chemax DNP-8®, and Soybean oil.

Another embodiment comprises cyclosporin A, Chemax DNP-18®, and Soybean oil.

Another embodiment comprises cyclosporin A, Chemax,DNP-150/50®, and Soybean oil.

Another embodiment comprises cyclosporin A, DeSonic 1.5N®, and Soybean oil.

Another embodiment comprises cyclosporin A, DeSonic 4N®, and Soybean oil.

Another embodiment comprises cyclosporin A, DeSonic 5N®, and Soybean oil.

Another embodiment comprises cyclosporin A, DeSonic 6D®, and Soybean oil.

Another embodiment comprises cyclosporin A, DeSonic 6N®, and Soybean oil.

Another embodiment comprises cyclosporin A, DeSonic 7N®, and Soybean oil.

Another embodiment comprises cyclosporin A, DeSonic 9N®, and Soybean oil.

Another embodiment comprises cyclosporin A, DeSonic 10D®, and Soybean oil.

Another embodiment comprises cyclosporin A, DeSonic 11N®, and Soybean oil.

Another embodiment comprises cyclosporin A, DeSonic 12N®, and Soybean oil.

Another embodiment comprises cyclosporin A, DeSonic 15N®, and Soybean oil.

Another embodiment comprises cyclosporin A, DeSonic 20N®, and Soybean oil.

Another embodiment comprises cyclosporin A, Eccoscour RC®, and Soybean oil.

Another embodiment comprises cyclosporin A, Eccoterge EO-100®, and Soybean oil.

Another embodiment comprises cyclosporin A, Emulsifier 632/90%®, and Soybean oil.

Another embodiment comprises cyclosporin A, Geronol AG-821®, and Soybean oil.

Another embodiment comprises cyclosporin A, Gradonic N-95®, and Soybean oil.

Another embodiment comprises cyclosporin A, Hetoxide NP-4®, and Soybean oil.

Another embodiment comprises cyclosporin A, Hetoxide NP-30®, and Soybean oil.

Another embodiment comprises cyclosporin A, Hostapal N-100®, and Soybean oil.

Another embodiment comprises cyclosporin A, Hostapal N-110®, and Soybean oil.

Another embodiment comprises cyclosporin A, Igepal CTA-639W®, and Soybean oil.

Another embodiment comprises cyclosporin A, Igepal DAP-9®, and Soybean oil.

Another embodiment comprises cyclosporin A, Igepal OD-410®, and Soybean oil.

Another embodiment comprises cyclosporin A, Igepal SS-837®, and Soybean oil.

Another embodiment comprises cyclosporin A, Lipocol NP-9 USP®, and Soybean oil.

Another embodiment comprises cyclosporin A, Macol DNP-10®, and Soybean oil.

Another embodiment comprises cyclosporin A, Marlophen NP 5®, and Soybean oil.

Another embodiment comprises cyclosporin A, Marlophen P 1®, and Soybean oil.

Another embodiment comprises cyclosporin A, Sunonic NB®, and Soybean oil.

Another embodiment comprises cyclosporin A, Surtonic OPB-307®, and Soybean oil.

Another embodiment comprises cyclosporin A, Surfonic OPB-407®, and Soybean oil.

Another embodiment comprises cyclosporin A, Syn Fac 334®, and Soybean oil.

Another embodiment comprises cyclosporin A, Syn Fac 8216®, and Soybean oil.

Another embodiment comprises cyclosporin A, Triton N-57®, and Soybean oil.

Another embodiment comprises cyclosporin A, Trycol 6956®, and Soybean oil.

Another embodiment comprises cyclosporin A, Trycol 6961®, and Soybean oil.

Another embodiment comprises cyclosporin A, Trycol 6964®, and Soybean oil.

Another embodiment comprises cyclosporin A, Trycol 6969®, and Soybean oil.

Another embodiment comprises cyclosporin A, Trycol 6974®, and Soybean oil.

Another embodiment comprises cyclosporin A, Witbreak DRB-127®, and Soybean oil.

Another embodiment comprises cyclosporin A, Witbreak DRB-127®, and Soybean oil.

Another embodiment comprises cyclosporin A, Witconol NP Series®, and Soybean oil.

Another embodiment comprises cyclosporin A, an Ethoxylated Aryl Phenol, and Soybean oil.

Another embodiment comprises cyclosporin A, Soprophor BSU®, and Soybean oil.

Another embodiment comprises cyclosporin A, Soprophor CY/8®, and Soybean oil.

Another embodiment comprises cyclosporin A, Soprophor S/25®, and Soybean oil.

Another embodiment comprises cyclosporin A, Witconol NIO®, and Soybean oil.

Another embodiment comprises cyclosporin A, Witconol NIW®, and Soybean oil.

Another embodiment comprises cyclosporin A, Witconol S-100®, and Soybean oil.

Another embodiment comprises cyclosporin A, an Ethoxylated Fatty Acid, and Soybean oil.

Another embodiment comprises cyclosporin A, Aldo PGHMS®, and Soybean oil.

Another embodiment comprises cyclosporin A, Alkamuls TO-15/HR®, and Soybean oil.

Another embodiment comprises cyclosporin A, Armotan AL-69-66®, and Soybean oil.

Another embodiment comprises cyclosporin A, Cerasynt 840®, and Soybean oil.

Another embodiment comprises cyclosporin A, Cerasynt 945®, and Soybean oil.

Another embodiment comprises cyclosporin A, Crystal Inhibitor No. 5®, and Soybean oil.

Another embodiment comprises cyclosporin A, DeThox Acid L-9®, and Soybean oil.

Another embodiment comprises cyclosporin A, DeThox Acid S-8®, and Soybean oil.

Another embodiment comprises cyclosporin A, Ethofat 242/25®, and Soybean oil.

Another embodiment comprises cyclosporin A, Hydropalat 65®, and Soybean oil.

Another embodiment comprises cyclosporin A, Lipo EGMS®, and Soybean oil.

Another embodiment comprises cyclosporin A, Lipopeg 2 DL®, and Soybean oil.

Another embodiment comprises cyclosporin A, Lipopeg 4 DL®, and Soybean oil.

Another embodiment comprises cyclosporin A, Lipopeg 4-L®, and Soybean oil.

Another embodiment comprises cyclosporin A, Lipopeg 39-S®, and Soybean oil.

Another embodiment comprises cyclosporin A, Lipopeg 4-S®, and Soybean oil.

Another embodiment comprises cyclosporin A, Lipopeg 10-S®, and Soybean oil.

Another embodiment comprises cyclosporin A, Lipopeg 100-S®, and Soybean oil.

Another embodiment comprises cyclosporin A, Lipopeg 6000 DS®, and Soybean oil.

Another embodiment comprises cyclosporin A, Lumulse 40-L®, and Soybean oil.

Another embodiment comprises cyclosporin A, Lumulse 40-S®, and Soybean oil.

Another embodiment comprises cyclosporin A, Lumulse 42-L®, and Soybean oil.

Another embodiment comprises cyclosporin A, Lumulse 42-S®, and Soybean oil.

Another embodiment comprises cyclosporin A, Lumulse 100-S®, and Soybean oil.

Another embodiment comprises cyclosporin A, Lumulse 602-S®, and Soybean oil.

Another embodiment comprises cyclosporin A, Magrabar PGE-20-0®, and Soybean oil.

Another embodiment comprises cyclosporin A, Magrabar PGE-20L®, and Soybean oil.

Another embodiment comprises cyclosporin A, Magrabar PGE-20T®, and Soybean oil.

Another embodiment comprises cyclosporin A, Magrabar PGE-22-0®, and Soybean oil.

Another embodiment comprises cyclosporin A, Magrabar PGE-22L®, and Soybean oil.

Another embodiment comprises cyclosporin A, Magrabar PGE-22T®, and Soybean oil.

Another embodiment comprises cyclosporin A, Magrabar PGE-40-0®, and Soybean oil.

Another embodiment comprises cyclosporin A, Magrabar PGE-40L®, and Soybean oil.

Another embodiment comprises cyclosporin A, Magrabar PGE-40T®, and Soybean oil.

Another embodiment comprises cyclosporin A, Magrabar PGE-42-0®, and Soybean oil.

Another embodiment comprises cyclosporin A, Magrabar PGE-42L®, and Soybean oil.

Another embodiment comprises cyclosporin A, Magrabar PGE-42T®, and Soybean oil.

Another embodiment comprises cyclosporin A, Magrabar PGE-60-0®, and Soybean oil.

Another embodiment comprises cyclosporin A, Magrabar PGE-60L®, and Soybean oil.

Another embodiment comprises cyclosporin A, Magrabar PGE-60T®, and Soybean oil.

Another embodiment comprises cyclosporin A, Magrabar PGE-62-0®, and Soybean oil.

Another embodiment comprises cyclosporin A, Magrabar PGE-62L®, and Soybean oil.

Another embodiment comprises cyclosporin A, Magrabar PGE-62T®, and Soybean oil.

Another embodiment comprises cyclosporin A, Mapeg S-40K®, and Soybean oil.

Another embodiment comprises cyclosporin A, Marlowet OTS®, and Soybean oil.

Another embodiment comprises cyclosporin A, Naturechem PGR®, and Soybean oil.

Another embodiment comprises cyclosporin A, PG No. 4®, and Soybean oil.

Another embodiment comprises cyclosporin A, Renex 20®, and Soybean oil.

Another embodiment comprises cyclosporin A, Ritox 52®, and Soybean oil.

Another embodiment comprises cyclosporin A, Ritox 53®, and Soybean oil.

Another embodiment comprises cyclosporin A, Ritox 59®, and Soybean oil.

Another embodiment comprises cyclosporin A, Surfax 8916/A®, and Soybean oil.

Another embodiment comprises cyclosporin A, Tego Acid S 40 P®, and Soybean oil.

Another embodiment comprises cyclosporin A, Tego Acid S 100 P®, and Soybean oil.

Another embodiment comprises cyclosporin A, Tween 20®, and Soybean oil.

Another embodiment comprises cyclosporin A, Volpo 131®, and Soybean oil.

Another embodiment comprises cyclosporin A, an Ethoxylated Fatty Ester or Oil, and Soybean oil.

Another embodiment comprises cyclosporin A, Acconon 6-C10®, and Soybean oil.

Another embodiment comprises cyclosporin A, Acconon CC-6®, and Soybean oil.

Another embodiment comprises cyclosporin A, Acconon CO-7®, and Soybean oil.

Another embodiment comprises cyclosporin A, Aldosperse 40/60 FG®, and Soybean oil.

Another embodiment comprises cyclosporin A, Aldosperse ML-23®, and Soybean oil.

Another embodiment comprises cyclosporin A, Aldosperse MS-20 FG®, and Soybean oil.

Another embodiment comprises cyclosporin A, Alkamuls EL-620®, and Soybean oil.

Another embodiment comprises cyclosporin A, Alkamuls EL-719®, and Soybean oil.

Another embodiment comprises cyclosporin A, Alkamuls EL-985®, and Soybean oil.

Another embodiment comprises cyclosporin A, Arlatone G®, and Soybean oil.

Another embodiment comprises cyclosporin A, Arlatone T®, and Soybean oil.

Another embodiment comprises cyclosporin A, Atlas G-1045A®, and Soybean oil.

Another embodiment comprises cyclosporin A, Atlas G-1086®, and Soybean oil.

Another embodiment comprises cyclosporin A, Atlas G-1087®, and Soybean oil.

Another embodiment comprises cyclosporin A, Atlas G-1089®, and Soybean oil.

Another embodiment comprises cyclosporin A, Atlas G-1096®, and Soybean oil.

Another embodiment comprises cyclosporin A, Atlas G-1292®, and Soybean oil.

Another embodiment comprises cyclosporin A, Atlas G-1293®, and Soybean oil.

Another embodiment comprises cyclosporin A, Atlas G-1300®, and Soybean oil.

Another embodiment comprises cyclosporin A, Atlas G-7076®, and Soybean oil.

Another embodiment comprises cyclosporin A, Capmul EMG®, and Soybean oil.

Another embodiment comprises cyclosporin A, Chemonic CO-40®, and Soybean oil.

Another embodiment comprises cyclosporin A, Chemonic LI-3®, and Soybean oil.

Another embodiment comprises cyclosporin A, Chemonic LI-7®, and Soybean oil.

Another embodiment comprises cyclosporin A, Cirrasol GM®, and Soybean oil.

Another embodiment comprises cyclosporin A, Cremophor CO 40®, and Soybean oil.

Another embodiment comprises cyclosporin A, Cremophor CO 410®, and Soybean oil.

Another embodiment comprises cyclosporin A, Cremophor EL®, and Soybean oil.

Another embodiment comprises cyclosporin A, Cremophor GC7®, and Soybean oil.

Another embodiment comprises cyclosporin A, Cremophor RH-40®, and Soybean oil.

Another embodiment comprises cyclosporin A, Crovol A-40®, and Soybean oil.

Another embodiment comprises cyclosporin A, Crovol A-70®, and Soybean oil.

Another embodiment comprises cyclosporin A, Crovol M-70®, and Soybean oil.

Another embodiment comprises cyclosporin A, Crovol PK-70®, and Soybean oil.

Another embodiment comprises cyclosporin A, Cutina E-24®, and Soybean oil.

Another embodiment comprises cyclosporin A, Dacospin 12-R®, and Soybean oil.

Another embodiment comprises cyclosporin A, Dehymuls HRE-7®, and Soybean oil.

Another embodiment comprises cyclosporin A, DeSonic 30C®, and Soybean oil.

Another embodiment comprises cyclosporin A, DeSonic 36C®, and Soybean oil.

Another embodiment comprises cyclosporin A, DeSonic 40C®, and Soybean oil.

Another embodiment comprises cyclosporin A, Durfax 60®, and Soybean oil.

Another embodiment comprises cyclosporin A, Durfax 65®, and Soybean oil.

Another embodiment comprises cyclosporin A, Durfax 80®, and Soybean oil.

Another embodiment comprises cyclosporin A, Durfax EOM®, and Soybean oil.

Another embodiment comprises cyclosporin A, Eccoterge NF-2®, and Soybean oil.

Another embodiment comprises cyclosporin A, Emulpon CO-360®, and Soybean oil.

Another embodiment comprises cyclosporin A, Emulpon CO-550®, and Soybean oil.

Another embodiment comprises cyclosporin A, Emulsogen EL®, and Soybean oil.

Another embodiment comprises cyclosporin A, Emulsogen HCO 040®, and Soybean oil.

Another embodiment comprises cyclosporin A, Emulsogen HCO 060®, and Soybean oil.

Another embodiment comprises cyclosporin A, Emulsynt 1055®, and Soybean oil.

Another embodiment comprises cyclosporin A, Ethox 3095®, and Soybean oil.

Another embodiment comprises cyclosporin A, Eumulgin RO-40®, and Soybean oil.

Another embodiment comprises cyclosporin A, Genapol G-260®, and Soybean oil.

Another embodiment comprises cyclosporin A, Glycosperse L-20®, and Soybean oil.

Another embodiment comprises cyclosporin A, Glycosperse O-5®, and Soybean oil.

Another embodiment comprises cyclosporin A, Glycosperse O-20®, and Soybean oil.

Another embodiment comprises cyclosporin A, Glycosperse O-20 FG®, and Soybean oil.

Another embodiment comprises cyclosporin A, Glycosperse S-20®, and Soybean oil.

Another embodiment comprises cyclosporin A, Glycosperse S-20 FG®, and Soybean oil.

Another embodiment comprises cyclosporin A, Glycosperse TS-20®, and Soybean oil.

Another embodiment comprises cyclosporin A, Glycosperse TS-20 FG®, and Soybean oil.

Another embodiment comprises cyclosporin A, Hetan SL®, and Soybean oil.

Another embodiment comprises cyclosporin A, Hetan SO®, and Soybean oil.

Another embodiment comprises cyclosporin A, Hetan SS®, and Soybean oil.

Another embodiment comprises cyclosporin A, Hetoxide C-2®, and Soybean oil.

Another embodiment comprises cyclosporin A, Hetoxide C-9®, and Soybean oil.

Another embodiment comprises cyclosporin A, Hetoxide C-15®, and Soybean oil.

Another embodiment comprises cyclosporin A, Hetoxide C-25®, and Soybean oil.

Another embodiment comprises cyclosporin A, Hetoxide C-40®, and Soybean oil.

Another embodiment comprises cyclosporin A, Hetoxide C-20®, and Soybean oil.

Another embodiment comprises cyclosporin A, Hetoxide C-200-50%®, and Soybean oil.

Another embodiment comprises cyclosporin A, Hetoxide GC-30®, and Soybean oil.

Another embodiment comprises cyclosporin A, Hetoxide HC-60®, and Soybean oil.

Another embodiment comprises cyclosporin A, Ice No. 2®, and Soybean oil.

Another embodiment comprises cyclosporin A, Incrocas 30/40®, and Soybean oil.

Another embodiment comprises cyclosporin A, Lexol EC®, and Soybean oil.

Another embodiment comprises cyclosporin A, Lexol EO®, and Soybean oil.

Another embodiment comprises cyclosporin A, Lipocol HCO-40®, and Soybean oil.

Another embodiment comprises cyclosporin A, Lipocol HCO-60®, and Soybean oil.

Another embodiment comprises cyclosporin A, Lipocol O-3 Special®, and Soybean oil.

Another embodiment comprises cyclosporin A, Lipopeg 2-L®, and Soybean oil.

Another embodiment comprises cyclosporin A, Lipopeg 4-DO®, and Soybean oil.

Another embodiment comprises cyclosporin A, Lipopeg 4-DS®, and Soybean oil.

Another embodiment comprises cyclosporin A, Lipovol GTB®, and Soybean oil.

Another embodiment comprises cyclosporin A, Lonzest SML-20®, and Soybean oil.

Another embodiment comprises cyclosporin A, Lonzest SMO-20®, and Soybean oil.

Another embodiment comprises cyclosporin A, Lonzest SMS-20®, and Soybean oil.

Another embodiment comprises cyclosporin A, Lonzest STO-20®, and Soybean oil.

Another embodiment comprises cyclosporin A, Lonzest STS-20®, and Soybean oil.

Another embodiment comprises cyclosporin A, Lumulse GR-40®, and Soybean oil.

Another embodiment comprises cyclosporin A, Lumulse GRH-40®, and Soybean oil.

Another embodiment comprises cyclosporin A, Lumulse POE (7) GML®, and Soybean oil.

Another embodiment comprises cyclosporin A, Lumulse POE (12) Glyc®, and Soybean oil.

Another embodiment comprises cyclosporin A, Lumulse POE (40) MS KP®, and Soybean oil.

Another embodiment comprises cyclosporin A, Marlowet 475®, and Soybean oil.

Another embodiment comprises cyclosporin A, Marlowet LVS®, and Soybean oil.

Another embodiment comprises cyclosporin A, Marlowet R 11®, and Soybean oil.

Another embodiment comprises cyclosporin A, Marlowet R 40®, and Soybean oil.

Another embodiment comprises cyclosporin A, Mazol 80 MGK®, and Soybean oil.

Another embodiment comprises cyclosporin A, Nonionic Emulsifier T-9®, and Soybean oil.

Another embodiment comprises cyclosporin A, Oronal LCG®, and Soybean oil.

Another embodiment comprises cyclosporin A, Polyderm PPI-CO-200®, and Soybean oil.

Another embodiment comprises cyclosporin A, Polyderm PPI-CO-40®, and Soybean oil.

Another embodiment comprises cyclosporin A, Rewoderm LI 520-70®, and Soybean oil.

Another embodiment comprises cyclosporin A, Ritapeg 150 DS®, and Soybean oil.

Another embodiment comprises cyclosporin A, Softigen 767®, and Soybean oil.

Another embodiment comprises cyclosporin A, Surfactol 318®, and Soybean oil.

Another embodiment comprises cyclosporin A, Surfactol 365®, and Soybean oil.

Another embodiment comprises cyclosporin A, Syn Lube 107®, and Soybean oil.

Another embodiment comprises cyclosporin A, Syn Lube 728®, and Soybean oil.

Another embodiment comprises cyclosporin A, Syn Lube 1632H®, and Soybean oil.

Another embodiment comprises cyclosporin A, Syn Lube 6277-A®, and Soybean oil.

Another embodiment comprises cyclosporin A, T-Det C-20®, and Soybean oil.

Another embodiment comprises cyclosporin A, T-Det C-40®, and Soybean oil.

Another embodiment comprises cyclosporin A, Tally 100 Plus®, and Soybean oil.

Another embodiment comprises cyclosporin A, Uniperol EL®, and Soybean oil.

Another embodiment comprises cyclosporin A, a Fatty Ester, and Soybean oil.

Another embodiment comprises cyclosporin A, Actralube-Syn 147®, and Soybean oil.

Another embodiment comprises cyclosporin A, Atlas G-1556®, and Soybean oil.

Another embodiment comprises cyclosporin A, Atlas G-1564®, and Soybean oil.

Another embodiment comprises cyclosporin A, Atlasol Base Oil S®, and Soybean oil.

Another embodiment comprises cyclosporin A, Base ML®, and Soybean oil.

Another embodiment comprises cyclosporin A, Base MT®, and Soybean oil.

Another embodiment comprises cyclosporin A, Cerasynt 303®, and Soybean oil.

Another embodiment comprises cyclosporin A, Dermol 1012®, and Soybean oil.

Another embodiment comprises cyclosporin A, Kemester 4000®, and Soybean oil.

Another embodiment comprises cyclosporin A, Lactipol S®, and Soybean oil.

Another embodiment comprises cyclosporin A, Magrabar PGO®, and Soybean oil.

Another embodiment comprises cyclosporin A, Mayco Base BFO®, and Soybean oil.

Another embodiment comprises cyclosporin A, Methyl Linoleate®, and Soybean oil.

Another embodiment comprises cyclosporin A, Pationic 122A®, and Soybean oil.

Another embodiment comprises cyclosporin A, Pationic 138C®, and Soybean oil.

Another embodiment comprises cyclosporin A, Pationic CSL®, and Soybean oil.

Another embodiment comprises cyclosporin A, Pationic ISL®, and Soybean oil.

Another embodiment comprises cyclosporin A, Pationic SBL®, and Soybean oil.

Another embodiment comprises cyclosporin A, Pationic SSL®, and Soybean oil.

Another embodiment comprises cyclosporin A, Ritasol®, and Soybean oil.

Another embodiment comprises cyclosporin A, Tego Alkanol CS 20®, and Soybean oil.

Another embodiment comprises cyclosporin A, Tego Alkanol L23 P®, and Soybean oil.

Another embodiment comprises cyclosporin A, Tego Alkanol S2®, and Soybean oil.

Another embodiment comprises cyclosporin A, Tego Alkanol S20 P®, and Soybean oil.

Another embodiment comprises cyclosporin A, Triemulsifier 600 MS®, and Soybean oil.

Another embodiment comprises cyclosporin A, a Glycerol Esters, and Soybean oil.

Another embodiment comprises cyclosporin A, Agro #9 Wint SBO®, and Soybean oil.

Another embodiment comprises cyclosporin A, Ahcovel Base 700®, and Soybean oil.

Another embodiment comprises cyclosporin A, Aldo HMS FG®, and Soybean oil.

Another embodiment comprises cyclosporin A, Aldo MLD®, and Soybean oil.

Another embodiment comprises cyclosporin A, Aldo MLD FG®, and Soybean oil.

Another embodiment comprises cyclosporin A, Aldo MO FG®, and Soybean oil.

Another embodiment comprises cyclosporin A, Aldo MS®, and Soybean oil.

Another embodiment comprises cyclosporin A, Aldo MS FG®, and Soybean oil.

Another embodiment comprises cyclosporin A, Aldo MS LG FG®, and Soybean oil.

Another embodiment comprises cyclosporin A, Aldo MSD®, and Soybean oil.

Another embodiment comprises cyclosporin A, Aldo MSD FG®, and Soybean oil.

Another embodiment comprises cyclosporin A, Aldosperse O-20 FG®, and Soybean oil.

Another embodiment comprises cyclosporin A, Aldosperse TS-20 FG®, and Soybean oil.

Another embodiment comprises cyclosporin A, Aldosperse TS-40 FG®, and Soybean oil.

Another embodiment comprises cyclosporin A, Arlacel 165®, and Soybean oil.

Another embodiment comprises cyclosporin A, Arlacel 186®, and Soybean oil.

Another embodiment comprises cyclosporin A, Capmul GMO®, and Soybean oil.

Another embodiment comprises cyclosporin A, Capmul GMS®, and Soybean oil.

Another embodiment comprises cyclosporin A, Caprol 3GO®, and Soybean oil.

Another embodiment comprises cyclosporin A, Caprol 3GVS®, and Soybean oil.

Another embodiment comprises cyclosporin A, Caprol 6G2S®, and Soybean oil.

Another embodiment comprises cyclosporin A, Caprol 10G40®, and Soybean oil.

Another embodiment comprises cyclosporin A, Caprol 10G100®, and Soybean oil.

Another embodiment comprises cyclosporin A, Caprol ET®, and Soybean oil.

Another embodiment comprises cyclosporin A, Caprol PGE860®, and Soybean oil.

Another embodiment comprises cyclosporin A, Cerasynt 945®, and Soybean oil.

Another embodiment comprises cyclosporin A, Cerasynt GMS®, and Soybean oil.

Another embodiment comprises cyclosporin A, Cerasynt Q®, and Soybean oil.

Another embodiment comprises cyclosporin A, Cerasynt SD®, and Soybean oil.

Another embodiment comprises cyclosporin A, Cerasynt WM®, and Soybean oil.

Another embodiment comprises cyclosporin A, Chemsperse 14®, and Soybean oil.

Another embodiment comprises cyclosporin A, Cremophor GO-32®, and Soybean oil.

Another embodiment comprises cyclosporin A, Cremophor GS11l®, and Soybean oil.

Another embodiment comprises cyclosporin A, Cremophor GS-32®, and Soybean oil.

Another embodiment comprises cyclosporin A, Cutina KD-16®, and Soybean oil.

Another embodiment comprises cyclosporin A, Dehymuls PGPH®, and Soybean oil.

Another embodiment comprises cyclosporin A, Dermol DGDIS®, and Soybean oil.

Another embodiment comprises cyclosporin A, Dermol DGMIS®, and Soybean oil.

Another embodiment comprises cyclosporin A, Dermol G-76®, and Soybean oil.

Another embodiment comprises cyclosporin A, Dermol G-7DI®, and Soybean oil.

Another embodiment comprises cyclosporin A, Dermol NGDI®, and Soybean oil.

Another embodiment comprises cyclosporin A, Dermolan GLH®, and Soybean oil.

Another embodiment comprises cyclosporin A, Drewmulse GMO®, and Soybean oil.

Another embodiment comprises cyclosporin A, Drewpol 3-5-M®, and Soybean oil.

Another embodiment comprises cyclosporin A, Durlac 100 W®, and Soybean oil.

Another embodiment comprises cyclosporin A, Dur-Lo®, and Soybean oil.

Another embodiment comprises cyclosporin A, Dynasan 118®, and Soybean oil.

Another embodiment comprises cyclosporin A, EC-25®, and Soybean oil.

Another embodiment comprises cyclosporin A, EM 40®, and Soybean oil.

Another embodiment comprises cyclosporin A, Emerest 240®, and Soybean oil.

Another embodiment comprises cyclosporin A, Emerest 2452®, and Soybean oil.

Another embodiment comprises cyclosporin A, Empilan G-26®, and Soybean oil.

Another embodiment comprises cyclosporin A, Genapol TSM®, and Soybean oil.

Another embodiment comprises cyclosporin A, Hostacerin DGI®, and Soybean oil.

Another embodiment comprises cyclosporin A, Hostacerin DGL®, and Soybean oil.

Another embodiment comprises cyclosporin A, Hostacerin DGMS®, and Soybean oil.

Another embodiment comprises cyclosporin A, Hostacerin DGSB®, and Soybean oil.

Another embodiment comprises cyclosporin A, Ice No. 2®, and Soybean oil.

Another embodiment comprises cyclosporin A, Imwitor 742®, and Soybean oil.

Another embodiment comprises cyclosporin A, Imwitor 780 K®, and Soybean oil.

Another embodiment comprises cyclosporin A, Imwitor 960 Flakes®, and Soybean oil.

Another embodiment comprises cyclosporin A, Isolan GI 34®, and Soybean oil.

Another embodiment comprises cyclosporin A, Isolan GO 33®, and Soybean oil.

Another embodiment comprises cyclosporin A, Kemester 1000®, and Soybean oil.

Another embodiment comprises cyclosporin A, Kemester 2000®, and Soybean oil.

Another embodiment comprises cyclosporin A, Kemester 2000®, and Soybean oil.

Another embodiment comprises cyclosporin A, Kemester 6000SE®, and Soybean oil.

Another embodiment comprises cyclosporin A, Lamecreme DGE 18®, and Soybean oil.

Another embodiment comprises cyclosporin A, Lexemul 515®, and Soybean oil.

Another embodiment comprises cyclosporin A, Lexemul 561®, and Soybean oil.

Another embodiment comprises cyclosporin A, Lexemul AR®, and Soybean oil.

Another embodiment comprises cyclosporin A, Lexemul AS®, and Soybean oil.

Another embodiment comprises cyclosporin A, Lexemul GDL®, and Soybean oil.

Another embodiment comprises cyclosporin A, Lexemul T®, and Soybean oil.

Another embodiment comprises cyclosporin A, Lipomulse 165®, and Soybean oil.

Another embodiment comprises cyclosporin A, Lumulse GML K®, and Soybean oil.

Another embodiment comprises cyclosporin A, Lumulse GMO K®, and Soybean oil.

Another embodiment comprises cyclosporin A, Lumulse GMR K®, and Soybean oil.

Another embodiment comprises cyclosporin A, Lumulse GMT K®, and Soybean oil.

Another embodiment comprises cyclosporin A, Magrabar GMC®, and Soybean oil.

Another embodiment comprises cyclosporin A, Magrabar GMO-CK®, and Soybean oil.

Another embodiment comprises cyclosporin A, Magrabar MDG-5050®, and Soybean oil.

Another embodiment comprises cyclosporin A, Magrabar PGO-315®, and Soybean oil.

Another embodiment comprises cyclosporin A, Magrabar PGO-1010®, and Soybean oil.

Another embodiment comprises cyclosporin A, Mazol 300K®, and Soybean oil.

Another embodiment comprises cyclosporin A, Mazol GMO-K®, and Soybean oil.

Another embodiment comprises cyclosporin A, Mazol GMS-K®, and Soybean oil.

Another embodiment comprises cyclosporin A, Mazol PG031 -K®, and Soybean oil.

Another embodiment comprises cyclosporin A, Miglyol 812®, and Soybean oil.

Another embodiment comprises cyclosporin A, Norfox 165C®, and Soybean oil.

Another embodiment comprises cyclosporin A, Schercemol GMIS®, and Soybean oil.

Another embodiment comprises cyclosporin A, Tegin®, and Soybean oil.

Another embodiment comprises cyclosporin A, Tegin 4100 Pellets®, and Soybean oil.

Another embodiment comprises cyclosporin A, Tegin M Pellets®, and Soybean oil.

Another embodiment comprises cyclosporin A, Tegin OV®, and Soybean oil.

Another embodiment comprises cyclosporin A, Teginacid H®, and Soybean oil.

Another embodiment comprises cyclosporin A, Tego Cosmo P813®, and Soybean oil.

Another embodiment comprises cyclosporin A, Wickenol 535®, and Soybean oil.

Another embodiment comprises cyclosporin A, Witconol 14®, and Soybean oil.

Another embodiment comprises cyclosporin A, Witconol 14®, and Soybean oil.

Another embodiment comprises cyclosporin A, Witconol 14F®, and Soybean oil.

Another embodiment comprises cyclosporin A, Witconol 18L®, and Soybean oil.

Another embodiment comprises cyclosporin A, Witconol GOT®, and Soybean oil.

Another embodiment comprises cyclosporin A, Witconol MST®, and Soybean oil.

Another embodiment comprises cyclosporin A, Witconol RHT®, and Soybean oil.

Another embodiment comprises cyclosporin A, a Glycol Ester, and Soybean oil.

Another embodiment comprises cyclosporin A, Alkamuls 600 DO®, and Soybean oil.

Another embodiment comprises cyclosporin A, Alkamuls SEG®, and Soybean oil.

Another embodiment comprises cyclosporin A, Atlas EM-2®, and Soybean oil.

Another embodiment comprises cyclosporin A, Cerasynt IP®, and Soybean oil.

Another embodiment comprises cyclosporin A, Cerasynt M®, and Soybean oil.

Another embodiment comprises cyclosporin A, Cerasynt MN®, and Soybean oil.

Another embodiment comprises cyclosporin A, Cerasynt PA®, and Soybean oil.

Another embodiment comprises cyclosporin A, Chemsperse EGDS®, and Soybean oil.

Another embodiment comprises cyclosporin A, Chemsperse EGMS®, and Soybean oil.

Another embodiment comprises cyclosporin A, Colonial Monolaurin®, and Soybean oil.

Another embodiment comprises cyclosporin A, DeMuls SGE-95®, and Soybean oil.

Another embodiment comprises cyclosporin A, Eccoterge 200®, and Soybean oil.

Another embodiment comprises cyclosporin A, Emerest 2380®, and Soybean oil.

Another embodiment comprises cyclosporin A, Ethox 2610®, and Soybean oil.

Another embodiment comprises cyclosporin A, Ethox DO-9®, and Soybean oil.

Another embodiment comprises cyclosporin A, Ethox DO-14®, and Soybean oil.

Another embodiment comprises cyclosporin A, Ethox SO-9®, and Soybean oil.

Another embodiment comprises cyclosporin A, Fizul MD-318®, and Soybean oil.

Another embodiment comprises cyclosporin A, Genapol EGDS-VHP®, and Soybean oil.

Another embodiment comprises cyclosporin A, Genapol TS Powder®, and Soybean oil.

Another embodiment comprises cyclosporin A, Hostacerin WO®, and Soybean oil.

Another embodiment comprises cyclosporin A, Inversol 140®, and Soybean oil.

Another embodiment comprises cyclosporin A, Kemester 104®, and Soybean oil.

Another embodiment comprises cyclosporin A, Kemester 205®, and Soybean oil.

Another embodiment comprises cyclosporin A, Kemester 226®, and Soybean oil.

Another embodiment comprises cyclosporin A, Kemester 5221SE®, and Soybean oil.

Another embodiment comprises cyclosporin A, Kemester EGDS®, and Soybean oil.

Another embodiment comprises cyclosporin A, Lexemul EGDS®, and Soybean oil.

Another embodiment comprises cyclosporin A, Lexemul EGMS®, and Soybean oil.

Another embodiment comprises cyclosporin A, Lexemul P®, and Soybean oil.

Another embodiment comprises cyclosporin A, Lipo DGLS®, and Soybean oil.

Another embodiment comprises cyclosporin A, Lipo EGDS®, and Soybean oil.

Another embodiment comprises cyclosporin A, Lipo PGMS®, and Soybean oil.

Another embodiment comprises cyclosporin A, Liposorb S-4®, and Soybean oil.

Another embodiment comprises cyclosporin A, Liposorb TO-20®, and Soybean oil.

Another embodiment comprises cyclosporin A, Lumulse PGO®, and Soybean oil.

Another embodiment comprises cyclosporin A, Mackester EGDS®, and Soybean oil.

Another embodiment comprises cyclosporin A, Mackester EGMS®, and Soybean oil.

Another embodiment comprises cyclosporin A, Mackester GSTP®, and Soybean oil.

Another embodiment comprises cyclosporin A, Mackester Series®, and Soybean oil.

Another embodiment comprises cyclosporin A, Magrabar PDG-50®, and Soybean oil.

Another embodiment comprises cyclosporin A, Mapeg 6000 DS®, and Soybean oil.

Another embodiment comprises cyclosporin A, Marlowet 4702®, and Soybean oil.

Another embodiment comprises cyclosporin A, Monalube 305®, and Soybean oil.

Another embodiment comprises cyclosporin A, Monalube 310®, and Soybean oil.

Another embodiment comprises cyclosporin A, Monalube 315®, and Soybean oil.

Another embodiment comprises cyclosporin A, Monalube 320®, and Soybean oil.

Another embodiment comprises cyclosporin A, Monalube 325®, and Soybean oil.

Another embodiment comprises cyclosporin A, Monalube 330®, and Soybean oil.

Another embodiment comprises cyclosporin A, Naturechem PGHS®, and Soybean oil.

Another embodiment comprises cyclosporin A, Polycastorol PLO-840®, and Soybean oil.

Another embodiment comprises cyclosporin A, Polytex 10M®, and Soybean oil.

Another embodiment comprises cyclosporin A, Ritasynt IP®, and Soybean oil.

Another embodiment comprises cyclosporin A, Ross Chem PEG 600 DT®, and Soybean oil.

Another embodiment comprises cyclosporin A, Schercemol PGMS®, and Soybean oil.

Another embodiment comprises cyclosporin A, Sponto H-44C®, and Soybean oil.

Another embodiment comprises cyclosporin A, Tegin G®, and Soybean oil.

Another embodiment comprises cyclosporin A, Witbreak DGE-182®, and Soybean oil.

Another embodiment comprises cyclosporin A, Witbreak DGE-182®, and Soybean oil.

Another embodiment comprises cyclosporin A, Witbreak DRA-21®, and Soybean oil.

Another embodiment comprises cyclosporin A, Witbreak DRA-21®, and Soybean oil.

Another embodiment comprises cyclosporin A, Witbreak DRA-50®, and Soybean oil.

Another embodiment comprises cyclosporin A, Witbreak DRA-50®, and Soybean oil.

Another embodiment comprises cyclosporin A, Witconol F26-46®, and Soybean oil.

Another embodiment comprises cyclosporin A, Witconol H-32®, and Soybean oil.

Another embodiment comprises cyclosporin A, Witconol H-33®, and Soybean oil.

Another embodiment comprises cyclosporin A, Witconol H-35A®, and Soybean oil.

Another embodiment comprises cyclosporin A, Witconol RHP®, and Soybean oil.

Another embodiment comprises cyclosporin A, a Lanolin-based Derivative, and Soybean oil.

Another embodiment comprises cyclosporin A, Amerchol CAB®, and Soybean oil.

Another embodiment comprises cyclosporin A, Amerchol L-101®, and Soybean oil.

Another embodiment comprises cyclosporin A, Amerlate LFA-LO®, and Soybean oil.

Another embodiment comprises cyclosporin A, Amerlate P®, and Soybean oil.

Another embodiment comprises cyclosporin A, Barre Common Degras®, and Soybean oil.

Another embodiment comprises cyclosporin A, Cholesterol NF®, and Soybean oil.

Another embodiment comprises cyclosporin A, Crodalan AWS®, and Soybean oil.

Another embodiment comprises cyclosporin A, Crodalan LA®, and Soybean oil.

Another embodiment comprises cyclosporin A, Emery 1650®, and Soybean oil.

Another embodiment comprises cyclosporin A, Emery 1650®, and Soybean oil.

Another embodiment comprises cyclosporin A, Emery 1740®, and Soybean oil.

Another embodiment comprises cyclosporin A, Emery 1740®, and Soybean oil.

Another embodiment comprises cyclosporin A, Forlan 500®, and Soybean oil.

Another embodiment comprises cyclosporin A, Forlan L®, and Soybean oil.

Another embodiment comprises cyclosporin A, Laneto 50®, and Soybean oil.

Another embodiment comprises cyclosporin A, Laneto 100®, and Soybean oil.

Another embodiment comprises cyclosporin A, Laneto AWS®, and Soybean oil.

Another embodiment comprises cyclosporin A, Lanfrax 1776®, and Soybean oil.

Another embodiment comprises cyclosporin A, Lanfrax 1776®, and Soybean oil.

Another embodiment comprises cyclosporin A, Lanogel 21®, and Soybean oil.

Another embodiment comprises cyclosporin A, Lipolan®, and Soybean oil.

Another embodiment comprises cyclosporin A, Lipolan 31®, and Soybean oil.

Another embodiment comprises cyclosporin A, OHlan®, and Soybean oil.

Another embodiment comprises cyclosporin A, Polychol 5®, and Soybean oil.

Another embodiment comprises cyclosporin A, Polychol 15®, and Soybean oil.

Another embodiment comprises cyclosporin A, Ritacetyl®, and Soybean oil.

Another embodiment comprises cyclosporin A, Ritachol®, and Soybean oil.

Another embodiment comprises cyclosporin A, Ritahydrox®, and Soybean oil.

Another embodiment comprises cyclosporin A, Ritalafa®, and Soybean oil.

Another embodiment comprises cyclosporin A, Ritalan®, and Soybean oil.

Another embodiment comprises cyclosporin A, Ritalan AWS®, and Soybean oil.

Another embodiment comprises cyclosporin A, Ritalan C®, and Soybean oil.

Another embodiment comprises cyclosporin A, Ritawax®, and Soybean oil.

Another embodiment comprises cyclosporin A, Ritawax AEO®, and Soybean oil.

Another embodiment comprises cyclosporin A, Ritawax ALA®, and Soybean oil.

Another embodiment comprises cyclosporin A, Solan/Solan 50/Super Solan®, and Soybean oil.

Another embodiment comprises cyclosporin A, Super Hartolan/Hartolan®, and Soybean oil.

Another embodiment comprises cyclosporin A, Supersat AWS-4®, and Soybean oil.

Another embodiment comprises cyclosporin A, Supersat AWS-24®, and Soybean oil.

Another embodiment comprises cyclosporin A, a Lecithin or Lecithin Derivative, and Soybean oil.

Another embodiment comprises cyclosporin A, Alcolec®, and Soybean oil.

Another embodiment comprises cyclosporin A, Lecithin®, and Soybean oil.

Another embodiment comprises cyclosporin A, Lexin K®, and Soybean oil.

Another embodiment comprises cyclosporin A, Natipide®, and Soybean oil.

Another embodiment comprises cyclosporin A, a Lignin or Lignin Derivative, and Soybean oil.

Another embodiment comprises cyclosporin A, Diwatex XP 9®, and Soybean oil.

Another embodiment comprises cyclosporin A, Dynasperse LCD®, and Soybean oil.

Another embodiment comprises cyclosporin A, Indulin SAL®, and Soybean oil.

Another embodiment comprises cyclosporin A, Indulin W-1®, and Soybean oil.

Another embodiment comprises cyclosporin A, Indulin W-5®, and Soybean oil.

Another embodiment comprises cyclosporin A, Lignosol FTA®, and Soybean oil.

Another embodiment comprises cyclosporin A, Lignosol SFX-65®, and Soybean oil.

Another embodiment comprises cyclosporin A, Marasperse 52 CP®, and Soybean oil.

Another embodiment comprises cyclosporin A, Marasperse AG®, and Soybean oil.

Another embodiment comprises cyclosporin A, Marasperse CBOS-4®, and Soybean oil.

Another embodiment comprises cyclosporin A, Ufoxane 2®, and Soybean oil.

Another embodiment comprises cyclosporin A, a Methyl Ester, and Soybean oil.

Another embodiment comprises cyclosporin A, E.B. Cleaner AK®, and Soybean oil.

Another embodiment comprises cyclosporin A, Oleocal ME-70®, and Soybean oil.

Another embodiment comprises cyclosporin A, Trade Name Company®, and Soybean oil.

Another embodiment comprises cyclosporin A, Methyl Esters (cont'd)®, and Soybean oil.

Another embodiment comprises cyclosporin A, Oleocal ME-92®, and Soybean oil.

Another embodiment comprises cyclosporin A, Oleocal ME-112®, and Soybean oil.

Another embodiment comprises cyclosporin A, Oleocal ME-130®, and Soybean oil.

Another embodiment comprises cyclosporin A, a Monoglyceride or a Derivative thereof, and Soybean oil.

Another embodiment comprises cyclosporin A, Dynacet 211®, and Soybean oil.

Another embodiment comprises cyclosporin A, Hetsorb S-20®, and Soybean oil.

Another embodiment comprises cyclosporin A, Imwitor 191®, and Soybean oil.

Another embodiment comprises cyclosporin A, Imwitor 370®, and Soybean oil.

Another embodiment comprises cyclosporin A, Imwitor 375®, and Soybean oil.

Another embodiment comprises cyclosporin A, Imwitor 900®, and Soybean oil.

Another embodiment comprises cyclosporin A, Imwitor 945®, and Soybean oil.

Another embodiment comprises cyclosporin A, Imwitor 2020®, and Soybean oil.

Another embodiment comprises cyclosporin A, Kemester 5500®, and Soybean oil.

Another embodiment comprises cyclosporin A, Kemester 6000®, and Soybean oil.

Another embodiment comprises cyclosporin A, Magrabar GMC®, and Soybean oil.

Another embodiment comprises cyclosporin A, Magrabar GMO-CK®, and Soybean oil.

Another embodiment comprises cyclosporin A, Magrabar GPC-10®, and Soybean oil.

Another embodiment comprises cyclosporin A, Magrabar MDG-5050®, and Soybean oil.

Another embodiment comprises cyclosporin A, Monalube 335®, and Soybean oil.

Another embodiment comprises cyclosporin A, Monoglycerides Glyceryl Monestearate Archer Daniels Midland Company®, and Soybean oil.

Another embodiment comprises cyclosporin A, Rita GMS®, and Soybean oil.

Another embodiment comprises cyclosporin A, Ritamulse SCG®, and Soybean oil.

Another embodiment comprises cyclosporin A, Softigen 701®, and Soybean oil.

Another embodiment comprises cyclosporin A, Tally 100 Plus®, and Soybean oil.

Another embodiment comprises cyclosporin A, a Polyethylene Glycol, and Soybean oil.

Another embodiment comprises cyclosporin A, Emulgade PL 68/50®, and Soybean oil.

Another embodiment comprises cyclosporin A, Lumulse PEG®, and Soybean oil.

Another embodiment comprises cyclosporin A, Rhodasurf PEG-400®, and Soybean oil.

Another embodiment comprises cyclosporin A, Rhodasurf PEG-600®, and Soybean oil.

Another embodiment comprises cyclosporin A, Witconol PEG-400®, and Soybean oil.

Another embodiment comprises cyclosporin A, a Polymeric Surfactant, and Soybean oil.

Another embodiment comprises cyclosporin A, Acritamer PNC-EG®, and Soybean oil.

Another embodiment comprises cyclosporin A, Ag-Rho DEP-775®, and Soybean oil.

Another embodiment comprises cyclosporin A, APG 325N Glycoside®, and Soybean oil.

Another embodiment comprises cyclosporin A, Aristoflex AVC®, and Soybean oil.

Another embodiment comprises cyclosporin A, Aristoflex HMB®, and Soybean oil.

Another embodiment comprises cyclosporin A, Burco NPS-225®, and Soybean oil.

Another embodiment comprises cyclosporin A, Burco NPS-816®, and Soybean oil.

Another embodiment comprises cyclosporin A, Chemccinate 5603®, and Soybean oil.

Another embodiment comprises cyclosporin A, Cosmedia Guar C-261N®, and Soybean oil.

Another embodiment comprises cyclosporin A, Gantrez S-95®, and Soybean oil.

Another embodiment comprises cyclosporin A, Glucopon 220 UP®, and Soybean oil.

Another embodiment comprises cyclosporin A, Glucopon 225 DK®, and Soybean oil.

Another embodiment comprises cyclosporin A, Glucopon 425 N®, and Soybean oil.

Another embodiment comprises cyclosporin A, Glucopon 600 UP®, and Soybean oil.

Another embodiment comprises cyclosporin A, Glucopon 625 UP®, and Soybean oil.

Another embodiment comprises cyclosporin A, Pemulen 1621®, and Soybean oil.

Another embodiment comprises cyclosporin A, Pemulen 1622®, and Soybean oil.

Another embodiment comprises cyclosporin A, Pemulen TR-1®, and Soybean oil.

Another embodiment comprises cyclosporin A, Pemulen TR-2®, and Soybean oil.

Another embodiment comprises cyclosporin A, Plantacare 818®, and Soybean oil.

Another embodiment comprises cyclosporin A, Plantapon LGC Sorb®, and Soybean oil.

Another embodiment comprises cyclosporin A, Plantaren 1200N®, and Soybean oil.

Another embodiment comprises cyclosporin A, Plantaren 2000N®, and Soybean oil.

Another embodiment comprises cyclosporin A, Viscolam AT 64®, and Soybean oil.

Another embodiment comprises cyclosporin A, Viscolam AT 64P®, and Soybean oil.

Another embodiment comprises cyclosporin A, Viscolam AT 100®, and Soybean oil.

Another embodiment comprises cyclosporin A, Viscolam MAC 7®, and Soybean oil.

Another embodiment comprises cyclosporin A, Viscolam SMC 20®, and Soybean oil.

Another embodiment comprises cyclosporin A, Witbreak RTC-323®, and Soybean oil.

Another embodiment comprises cyclosporin A, WSI 3700®, and Soybean oil.

Another embodiment comprises cyclosporin A, a Propoxylated or Ethoxylated Fatty Acid, Alcohol, or Alkyl Phenol, and Soybean oil.

Another embodiment comprises cyclosporin A, Antarox AA-60®, and Soybean oil.

Another embodiment comprises cyclosporin A, Antarox LF-224®, and Soybean oil.

Another embodiment comprises cyclosporin A, Burcomul DFE-45®, and Soybean oil.

Another embodiment comprises cyclosporin A, Burcoterge LFE-1000®, and Soybean oil.

Another embodiment comprises cyclosporin A, Chemal LF-25B®, and Soybean oil.

Another embodiment comprises cyclosporin A, Chemal LF-40B®, and Soybean oil.

Another embodiment comprises cyclosporin A, Dehypon LS-36®, and Soybean oil.

Another embodiment comprises cyclosporin A, Dehypon LS-36®, and Soybean oil.

Another embodiment comprises cyclosporin A, Dehypon LS-54®, and Soybean oil.

Another embodiment comprises cyclosporin A, Dehypon LS-54®, and Soybean oil.

Another embodiment comprises cyclosporin A, Delonic 100 VLF®, and Soybean oil.

Another embodiment comprises cyclosporin A, Delonic LF-60 MOD®, and Soybean oil.

Another embodiment comprises cyclosporin A, Empiderm B®, and Soybean oil.

Another embodiment comprises cyclosporin A, Ethylan 1206®, and Soybean oil.

Another embodiment comprises cyclosporin A, Ethylan NS-500K®, and Soybean oil.

Another embodiment comprises cyclosporin A, Ethylan NS-500LQ®, and Soybean oil.

Another embodiment comprises cyclosporin A, Genapol 1392®, and Soybean oil.

Another embodiment comprises cyclosporin A, Genapol 2317®, and Soybean oil.

Another embodiment comprises cyclosporin A, Genapol 26EP710®, and Soybean oil.

Another embodiment comprises cyclosporin A, Genapol EP 1022®, and Soybean oil.

Another embodiment comprises cyclosporin A, Genapol EP 1024®, and Soybean oil.

Another embodiment comprises cyclosporin A, Genapol EP 6068®, and Soybean oil.

Another embodiment comprises cyclosporin A, Genapol NP915®, and Soybean oil.

Another embodiment comprises cyclosporin A, Kieralon MFB®, and Soybean oil.

Another embodiment comprises cyclosporin A, Lumisolve CSA-80 V®, and Soybean oil.

Another embodiment comprises cyclosporin A, Marlowet 5001®, and Soybean oil.

Another embodiment comprises cyclosporin A, Marlox FK 64®, and Soybean oil.

Another embodiment comprises cyclosporin A, Marlox MO 124®, and Soybean oil.

Another embodiment comprises cyclosporin A, Marlox S 58®, and Soybean oil.

Another embodiment comprises cyclosporin A, Nonatell 1003®, and Soybean oil.

Another embodiment comprises cyclosporin A, Nonatell 1038®, and Soybean oil.

Another embodiment comprises cyclosporin A, Nonatell 1052®, and Soybean oil.

Another embodiment comprises cyclosporin A, Nonatell 1061®, and Soybean oil.

Another embodiment comprises cyclosporin A, Nonatell 1075®, and Soybean oil.

Another embodiment comprises cyclosporin A, Nonatell 1088®, and Soybean oil.

Another embodiment comprises cyclosporin A, Nonatell 1123®, and Soybean oil.

Another embodiment comprises cyclosporin A, Nonatell 1153®, and Soybean oil.

Another embodiment comprises cyclosporin A, Nonatell 1161®, and Soybean oil.

Another embodiment comprises cyclosporin A, Nonatell 1172®, and Soybean oil.

Another embodiment comprises cyclosporin A, Nonatell 1181®, and Soybean oil.

Another embodiment comprises cyclosporin A, Norfox 36®, and Soybean oil.

Another embodiment comprises cyclosporin A, Procetyl AWS®, and Soybean oil.

Another embodiment comprises cyclosporin A, Sandoxylate SX 412®, and Soybean oil.

Another embodiment comprises cyclosporin A, Sandoxylate SX 418®, and Soybean oil.

Another embodiment comprises cyclosporin A, Surfonic JL-80X®, and Soybean oil.

Another embodiment comprises cyclosporin A, Surfonic JL-80X-B1®, and Soybean oil.

Another embodiment comprises cyclosporin A, Surfonic L4-29X®, and Soybean oil.

Another embodiment comprises cyclosporin A, Surfonic LF®, and Soybean oil.

Another embodiment comprises cyclosporin A, T-Det A826®, and Soybean oil.

Another embodiment comprises cyclosporin A, T-Det LF-416®, and Soybean oil.

Another embodiment comprises cyclosporin A, Tergitol Min-Foam 1X®, and Soybean oil.

Another embodiment comprises cyclosporin A, Tergitol Min-Foam 2X®, and Soybean oil.

Another embodiment comprises cyclosporin A, Triton CF-21®, and Soybean oil.

Another embodiment comprises cyclosporin A, Triton CF-76®, and Soybean oil.

Another embodiment comprises cyclosporin A, Triton XL-80N®, and Soybean oil.

Another embodiment comprises cyclosporin A, Witconol NS-98®, and Soybean oil.

Another embodiment comprises cyclosporin A, Witconol NS-108LQ®, and Soybean oil.

Another embodiment comprises cyclosporin A, Witconol NS-145®, and Soybean oil.

Another embodiment comprises cyclosporin A, Witconol NS-179®, and Soybean oil.

Another embodiment comprises cyclosporin A, a Protein-based Surfactant, and Soybean oil.

Another embodiment comprises cyclosporin A, AminoFoam W®, and Soybean oil.

Another embodiment comprises cyclosporin A, Amiter LGOD-2®, and Soybean oil.

Another embodiment comprises cyclosporin A, Amiter LGS-2®, and Soybean oil.

Another embodiment comprises cyclosporin A, Amiter LGS-5®, and Soybean oil.

Another embodiment comprises cyclosporin A, Lamepon S®, and Soybean oil.

Another embodiment comprises cyclosporin A, Lamepon S®, and Soybean oil.

Another embodiment comprises cyclosporin A, Maypon 4C®, and Soybean oil.

Another embodiment comprises cyclosporin A, May-Tein C®, and Soybean oil.

Another embodiment comprises cyclosporin A, May-Tein CT®, and Soybean oil.

Another embodiment comprises cyclosporin A, May-Tein KTS®, and Soybean oil.

Another embodiment comprises cyclosporin A, May-Tein SY®, and Soybean oil.

Another embodiment comprises cyclosporin A, Plantapon S®, and Soybean oil.

Another embodiment comprises cyclosporin A, Proteol APL®, and Soybean oil.

Another embodiment comprises cyclosporin A, Proteol OAT®, and Soybean oil.

Another embodiment comprises cyclosporin A, Pyroter CPI-4®, and Soybean oil.

Another embodiment comprises cyclosporin A, Pyroter GPI-25®, and Soybean oil.

Another embodiment comprises cyclosporin A, Supro-Tein S®, and Soybean oil.

Another embodiment comprises cyclosporin A, Supro-Tein V®, and Soybean oil.

Another embodiment comprises cyclosporin A,® and Soybean oil.

Another embodiment comprises cyclosporin A, a Sarcosine Derivative®, and Soybean oil.

Another embodiment comprises cyclosporin A, Crodasinic LS-30®, and Soybean oil.

Another embodiment comprises cyclosporin A, Vanseal CS®, and Soybean oil.

Another embodiment comprises cyclosporin A, Vanseal LS®, and Soybean oil.

Another embodiment comprises cyclosporin A, Vanseal MS®, and Soybean oil.

Another embodiment comprises cyclosporin A, Vanseal NACS-30®, and Soybean oil.

Another embodiment comprises cyclosporin A, Vanseal NALS-95®, and Soybean oil.

Another embodiment comprises cyclosporin A, Vanseal OS®, and Soybean oil.

Another embodiment comprises cyclosporin A,® and Soybean oil.

Another embodiment comprises cyclosporin A, a Silicone-based Surfactant®, and Soybean oil.

Another embodiment comprises cyclosporin A, Abil-B-995®, and Soybean oil.

Another embodiment comprises cyclosporin A, Abil Care 85®, and Soybean oil.

Another embodiment comprises cyclosporin A, Abil EM 90®, and Soybean oil.

Another embodiment comprises cyclosporin A, Abil EM 97®, and Soybean oil.

Another embodiment comprises cyclosporin A, Abil WE-09®, and Soybean oil.

Another embodiment comprises cyclosporin A, Dow Corning 1248 Fluid®, and Soybean oil.

Another embodiment comprises cyclosporin A, Dow Corning 3225C®, and Soybean oil.

Another embodiment comprises cyclosporin A, Dow Corning 5200®, and Soybean oil.

Another embodiment comprises cyclosporin A, Dow Corning Q4-3667®, and Soybean oil.

Another embodiment comprises cyclosporin A, Monasil PCA®, and Soybean oil.

Another embodiment comprises cyclosporin A, Monasil PDM®, and Soybean oil.

Another embodiment comprises cyclosporin A, Monasil PLN®, and Soybean oil.

Another embodiment comprises cyclosporin A, Polyderm PPI-SI-WS®, and Soybean oil.

Another embodiment comprises cyclosporin A, Troysol 380W®, and Soybean oil.

Another embodiment comprises cyclosporin A, Troysol S366®, and Soybean oil.

Another embodiment comprises cyclosporin A, a Sorbitan Derivative, and Soybean oil.

Another embodiment comprises cyclosporin A, Alkamuls SML®, and Soybean oil.

Another embodiment comprises cyclosporin A, Alkamuls SMO®, and Soybean oil.

Another embodiment comprises cyclosporin A, Alkamuls STO®, and Soybean oil.

Another embodiment comprises cyclosporin A, Arlacel 20®, and Soybean oil.

Another embodiment comprises cyclosporin A, Arlacel 40®, and Soybean oil.

Another embodiment comprises cyclosporin A, Arlacel 60®, and Soybean oil.

Another embodiment comprises cyclosporin A, Arlacel 80®, and Soybean oil.

Another embodiment comprises cyclosporin A, Arlacel C®, and Soybean oil.

Another embodiment comprises cyclosporin A, Armul 21®, and Soybean oil.

Another embodiment comprises cyclosporin A, Atlox 80®, and Soybean oil.

Another embodiment comprises cyclosporin A, Atlox 8472®, and Soybean oil.

Another embodiment comprises cyclosporin A, Atlox 1045A®, and Soybean oil.

Another embodiment comprises cyclosporin A, Canarcel 20®, and Soybean oil.

Another embodiment comprises cyclosporin A, Canarcel 60®, and Soybean oil.

Another embodiment comprises cyclosporin A, Canarcel 80®, and Soybean oil.

Another embodiment comprises cyclosporin A, Canarcel TW 20®, and Soybean oil.

Another embodiment comprises cyclosporin A, Canarcel TW 60®, and Soybean oil.

Another embodiment comprises cyclosporin A, Canarcel TW 80®, and Soybean oil.

Another embodiment comprises cyclosporin A, Coladet BSB-P®, and Soybean oil.

Another embodiment comprises cyclosporin A, Customulse O-20®, and Soybean oil.

Another embodiment comprises cyclosporin A, Dehymuls E®, and Soybean oil.

Another embodiment comprises cyclosporin A, DeSotan SMO®, and Soybean oil.

Another embodiment comprises cyclosporin A, DeSotan SMO-20®, and Soybean oil.

Another embodiment comprises cyclosporin A, DeSotan SMT®, and Soybean oil.

Another embodiment comprises cyclosporin A, DeSotan SMT-20®, and Soybean oil.

Another embodiment comprises cyclosporin A, Durfax 60®, and Soybean oil.

Another embodiment comprises cyclosporin A, Durfax 65®, and Soybean oil.

Another embodiment comprises cyclosporin A, Durfax 80®, and Soybean oil.

Another embodiment comprises cyclosporin A, Durtan 60®, and Soybean oil.

Another embodiment comprises cyclosporin A, Durtan 65®, and Soybean oil.

Another embodiment comprises cyclosporin A, Liposorb L®, and Soybean oil.

Another embodiment comprises cyclosporin A, Liposorb L-10®, and Soybean oil.

Another embodiment comprises cyclosporin A, Liposorb L-20®, and Soybean oil.

Another embodiment comprises cyclosporin A, Liposorb O®, and Soybean oil.

Another embodiment comprises cyclosporin A, Liposorb O-20®, and Soybean oil.

Another embodiment comprises cyclosporin A, Liposorb P®, and Soybean oil.

Another embodiment comprises cyclosporin A, Liposorb P-20®, and Soybean oil.

Another embodiment comprises cyclosporin A, Liposorb S®, and Soybean oil.

Another embodiment comprises cyclosporin A, Liposorb S-20®, and Soybean oil.

Another embodiment comprises cyclosporin A, Liposorb SQO®, and Soybean oil.

Another embodiment comprises cyclosporin A, Liposorb TO®, and Soybean oil.

Another embodiment comprises cyclosporin A, Liposorb TS®, and Soybean oil.

Another embodiment comprises cyclosporin A, Liposorb TS-20®, and Soybean oil.

Another embodiment comprises cyclosporin A, Lumisorb PS®, and Soybean oil.

Another embodiment comprises cyclosporin A, Lumisorb SMO (T)®, and Soybean oil.

Another embodiment comprises cyclosporin A, Lumisorb SMS K®, and Soybean oil.

Another embodiment comprises cyclosporin A, Lumisorb SSO®, and Soybean oil.

Another embodiment comprises cyclosporin A, Lumisorb STS K®, and Soybean oil.

Another embodiment comprises cyclosporin A, Lumisorb STT®, and Soybean oil.

Another embodiment comprises cyclosporin A, Magrabar SMO®, and Soybean oil.

Another embodiment comprises cyclosporin A, Magrabar SMO-VEG®, and Soybean oil.

Another embodiment comprises cyclosporin A, Magrabar SMT®, and Soybean oil.

Another embodiment comprises cyclosporin A, Magrabar STO®, and Soybean oil.

Another embodiment comprises cyclosporin A, Miracare BC-27®, and Soybean oil.

Another embodiment comprises cyclosporin A, Ritabate 20®, and Soybean oil.

Another embodiment comprises cyclosporin A, Ritabate 40®, and Soybean oil.

Another embodiment comprises cyclosporin A, Ritabate 60®, and Soybean oil.

Another embodiment comprises cyclosporin A, Ritabate 80®, and Soybean oil.

Another embodiment comprises cyclosporin A, T-Maz®, and Soybean oil.

Another embodiment comprises cyclosporin A, Tego SML®, and Soybean oil.

Another embodiment comprises cyclosporin A, Tego SML 20®, and Soybean oil.

Another embodiment comprises cyclosporin A, Tego SMO 80 V®, and Soybean oil.

Another embodiment comprises cyclosporin A, Tego SMO V®, and Soybean oil.

Another embodiment comprises cyclosporin A, Tego SMS®, and Soybean oil.

Another embodiment comprises cyclosporin A, Tego STO V®, and Soybean oil.

Another embodiment comprises cyclosporin A, Tween 21®, and Soybean oil.

Another embodiment comprises cyclosporin A, Tween 40®, and Soybean oil.

Another embodiment comprises cyclosporin A, Tween 60®, and Soybean oil.

Another embodiment comprises cyclosporin A, Tween 60 K®, and Soybean oil.

Another embodiment comprises cyclosporin A, Tween 61®, and Soybean oil.

Another embodiment comprises cyclosporin A, Tween 65®, and Soybean oil.

Another embodiment comprises cyclosporin A, Tween 80®, and Soybean oil.

Another embodiment comprises cyclosporin A, Tween 80 K®, and Soybean oil.

Another embodiment comprises cyclosporin A, Tween 81®, and Soybean oil.

Another embodiment comprises cyclosporin A, Tween 85®, and Soybean oil.

Another embodiment comprises cyclosporin A, a Sucrose or Glucose Ester, or Derivative thereof, and Soybean oil.

Another embodiment comprises cyclosporin A, DeSulf GOS-P-60WCG®, and Soybean oil.

Another embodiment comprises cyclosporin A, Glucam E-20 Distearate®, and Soybean oil.

Another embodiment comprises cyclosporin A, Glucamate DOE-120®, and Soybean oil.

Another embodiment comprises cyclosporin A, Glucamate SSE-20®, and Soybean oil.

Another embodiment comprises cyclosporin A, Glucate DO®, and Soybean oil.

Another embodiment comprises cyclosporin A, Glucate SS®, and Soybean oil.

Another embodiment comprises cyclosporin A, Glucopon 425 UP®, and Soybean oil.

Another embodiment comprises cyclosporin A, Isolan IS®, and Soybean oil.

Another embodiment comprises cyclosporin A, Mazon 40®, and Soybean oil.

Another embodiment comprises cyclosporin A, Montanov 82®, and Soybean oil.

Another embodiment comprises cyclosporin A, Montanov 202®, and Soybean oil.

Another embodiment comprises cyclosporin A, Montanov S®, and Soybean oil.

Another embodiment comprises cyclosporin A, Rheozan®, and Soybean oil.

Another embodiment comprises cyclosporin A, Simulsol AS 48®, and Soybean oil.

Another embodiment comprises cyclosporin A, Simulsol SL 4®, and Soybean oil.

Another embodiment comprises cyclosporin A, Simulsol SL 10®, and Soybean oil.

Another embodiment comprises cyclosporin A, Simulsol SL 11W®, and Soybean oil.

Another embodiment comprises cyclosporin A, Simulsol SL 55®, and Soybean oil.

Another embodiment comprises cyclosporin A, Suga Nate 100 and 160®, and Soybean oil.

Another embodiment comprises cyclosporin A, Tego Care 450®, and Soybean oil.

Another embodiment comprises cyclosporin A, Tego Care CG 90®, and Soybean oil.

Another embodiment comprises cyclosporin A, Tego Care PS®, and Soybean oil.

Another embodiment comprises cyclosporin A, Tegosoft PSE 141 G®, and Soybean oil.

Another embodiment comprises cyclosporin A, Tegotens G 826®, and Soybean oil.

Another embodiment comprises cyclosporin A, Triton BG-10 (70%)®, and Soybean oil.

Another embodiment comprises cyclosporin A, Triton CG-110 (60%)®, and Soybean oil.

Another embodiment comprises cyclosporin A, Wickenol 545®, and Soybean oil.

Another embodiment comprises cyclosporin A, an alcohol, and Olive oil.

Another embodiment comprises cyclosporin A, Diglycerols®, and Olive oil.

Another embodiment comprises cyclosporin A, Hetoxide GT-80®, and Olive oil.

Another embodiment comprises cyclosporin A, Lexemul BEO®, and Olive oil.

Another embodiment comprises cyclosporin A, Polyglycerol-3®, and Olive oil.

Another embodiment comprises cyclosporin A, Redicote E Series®, and Olive oil.

Another embodiment comprises cyclosporin A, Simulsol OX 1005L®, and Olive oil.

Another embodiment comprises cyclosporin A, Stanfax 567®, and Olive oil.

Another embodiment comprises cyclosporin A, TA-1618®, and Olive oil.

Another embodiment comprises cyclosporin A, Witconol H-31A®, and Olive oil.

Another embodiment comprises cyclosporin A, an amine oxide, and Olive oil.

Another embodiment comprises cyclosporin A, AO-405®, and Olive oil.

Another embodiment comprises cyclosporin A, AO-455®, and Olive oil.

Another embodiment comprises cyclosporin A, AO 728 Special®, and Olive oil.

Another embodiment comprises cyclosporin A, Barlox 12®, and Olive oil.

Another embodiment comprises cyclosporin A, Barlox 14®, and Olive oil.

Another embodiment comprises cyclosporin A, Burcoxide Lo®, and Olive oil.

Another embodiment comprises cyclosporin A, Caloxamine LO®, and Olive oil.

Another embodiment comprises cyclosporin A, Chemoxide CAW®, and Olive oil.

Another embodiment comprises cyclosporin A, Chemoxide LM-30®, and Olive oil.

Another embodiment comprises cyclosporin A, Chemoxide LO®, and Olive oil.

Another embodiment comprises cyclosporin A, Chemoxide MO®, and Olive oil.

Another embodiment comprises cyclosporin A, Colalux CAO-35®, and Olive oil.

Another embodiment comprises cyclosporin A, Colalux LO®, and Olive oil.

Another embodiment comprises cyclosporin A, DeMox CAPO®, and Olive oil.

Another embodiment comprises cyclosporin A, DeMox CSG-30®, and Olive oil.

Another embodiment comprises cyclosporin A, DeMox LAO®, and Olive oil.

Another embodiment comprises cyclosporin A, Emcol L®, and Olive oil.

Another embodiment comprises cyclosporin A, Empigen OB®, and Olive oil.

Another embodiment comprises cyclosporin A, Empigen OS/A®, and Olive oil.

Another embodiment comprises cyclosporin A, Foamox CDO®, and Olive oil.

Another embodiment comprises cyclosporin A, Foamox DMM®, and Olive oil.

Another embodiment comprises cyclosporin A, Foamox DMS®, and Olive oil.

Another embodiment comprises cyclosporin A, Genaminox KC®, and Olive oil.

Another embodiment comprises cyclosporin A, Genaminox LA®, and Olive oil.

Another embodiment comprises cyclosporin A, Hartofoam SAO®, and Olive oil.

Another embodiment comprises cyclosporin A, Hartox DMCD®, and Olive oil.

Another embodiment comprises cyclosporin A, Lipowax DAT®, and Olive oil.

Another embodiment comprises cyclosporin A, Lipowax PB Pastilles®, and Olive oil.

Another embodiment comprises cyclosporin A, Mackamine C8®, and Olive oil.

Another embodiment comprises cyclosporin A, Mackamine C10®, and Olive oil.

Another embodiment comprises cyclosporin A, Mackamine C14®, and Olive oil.

Another embodiment comprises cyclosporin A, Mackamine CAO®, and Olive oil.

Another embodiment comprises cyclosporin A, Mackamine CO®, and Olive oil.

Another embodiment comprises cyclosporin A, Mackamine LO®, and Olive oil.

Another embodiment comprises cyclosporin A, Mackamine O2®, and Olive oil.

Another embodiment comprises cyclosporin A, Mackamine SAO®, and Olive oil.

Another embodiment comprises cyclosporin A, Mackamine SO®, and Olive oil.

Another embodiment comprises cyclosporin A, Mazox KCAO®, and Olive oil.

Another embodiment comprises cyclosporin A, Monalac MO®, and Olive oil.

Another embodiment comprises cyclosporin A, Norfox LDA®, and Olive oil.

Another embodiment comprises cyclosporin A, Rhodamox LO®, and Olive oil.

Another embodiment comprises cyclosporin A, Schercamox C-AA®, and Olive oil.

Another embodiment comprises cyclosporin A, Schercamox DMA®, and Olive oil.

Another embodiment comprises cyclosporin A, Schercamox DML®, and Olive oil.

Another embodiment comprises cyclosporin A, Schercamox DMM®, and Olive oil.

Another embodiment comprises cyclosporin A, Schercamox DMS®, and Olive oil.

Another embodiment comprises cyclosporin A, Tegotens DO®, and Olive oil.

Another embodiment comprises cyclosporin A, Tomah AO-14-2®, and Olive oil.

Another embodiment comprises cyclosporin A, Triaminox CDO®, and Olive oil.

Another embodiment comprises cyclosporin A, a block polymer, and Olive oil.

Another embodiment comprises cyclosporin A, AL 2070®, and Olive oil.

Another embodiment comprises cyclosporin A, Antarox 17-R-2®, and Olive oil.

Another embodiment comprises cyclosporin A, Antarox 25-R-2®, and Olive oil.

Another embodiment comprises cyclosporin A, Antarox 31-R-1®, and Olive oil.

Another embodiment comprises cyclosporin A, Antarox P-84®, and Olive oil.

Another embodiment comprises cyclosporin A, Antarox P-104/H®, and Olive oil.

Another embodiment comprises cyclosporin A, Arnox BP-Series®, and Olive oil.

Another embodiment comprises cyclosporin A, Chemonic 435®, and Olive oil.

Another embodiment comprises cyclosporin A, Chemonic D-25®, and Olive oil.

Another embodiment comprises cyclosporin A, Chemonic PL Series®, and Olive oil.

Another embodiment comprises cyclosporin A, Ethox L-121®, and Olive oil.

Another embodiment comprises cyclosporin A, Ethox L-122®, and Olive oil.

Another embodiment comprises cyclosporin A, Genapol PF-10®, and Olive oil.

Another embodiment comprises cyclosporin A, Genapol PF-20®, and Olive oil.

Another embodiment comprises cyclosporin A, Genapol PF-40A®, and Olive oil.

Another embodiment comprises cyclosporin A, Norfox 2-LF®, and Olive oil.

Another embodiment comprises cyclosporin A, Simulsol NW 342®, and Olive oil.

Another embodiment comprises cyclosporin A, T-Det BP-1®, and Olive oil.

Another embodiment comprises cyclosporin A, T-Det XD®, and Olive oil.

Another embodiment comprises cyclosporin A, T-Det XH®, and Olive oil.

Another embodiment comprises cyclosporin A, Triton CF-32®, and Olive oil.

Another embodiment comprises cyclosporin A, Witconol 171®, and Olive oil.

Another embodiment comprises cyclosporin A, Witconol 324®, and Olive oil.

Another embodiment comprises cyclosporin A, Witconol 324D®, and Olive oil.

Another embodiment comprises cyclosporin A, Witconol PD-2000®, and Olive oil.

Another embodiment comprises cyclosporin A, a Carboxylated Alcohol or Alkylphenol Ethoxylate, and Olive oil.

Another embodiment comprises cyclosporin A, Emcol CN-6®, and Olive oil.

Another embodiment comprises cyclosporin A, Ethcarb®, and Olive oil.

Another embodiment comprises cyclosporin A, Gemtex WNT-Conc®, and Olive oil.

Another embodiment comprises cyclosporin A, Incrodet TD7-C®, and Olive oil.

Another embodiment comprises cyclosporin A, Marlinat CM 105/80®, and Olive oil.

Another embodiment comprises cyclosporin A, Marlowet 1072®, and Olive oil.

Another embodiment comprises cyclosporin A, Marlowet 4530®, and Olive oil.

Another embodiment comprises cyclosporin A, Marlowet 4530 LF®, and Olive oil.

Another embodiment comprises cyclosporin A, Marlowet 4534®, and Olive oil.

Another embodiment comprises cyclosporin A, Marlowet 4538®, and Olive oil.

Another embodiment comprises cyclosporin A, Marlowet 4539®, and Olive oil.

Another embodiment comprises cyclosporin A, Marlowet 4539 LF®, and Olive oil.

Another embodiment comprises cyclosporin A, Marlowet 4541®, and Olive oil.

Another embodiment comprises cyclosporin A, Miranate LEC-80®, and Olive oil.

Another embodiment comprises cyclosporin A, Sandopan B®, and Olive oil.

Another embodiment comprises cyclosporin A, Sandopan B Modified®, and Olive oil.

Another embodiment comprises cyclosporin A, Sandopan LS-24 Gel®, and Olive oil.

Another embodiment comprises cyclosporin A, Surfine T-A®, and Olive oil.

Another embodiment comprises cyclosporin A, a Carboxylic Acid/Fatty Acid, and Olive oil.

Another embodiment comprises cyclosporin A, Colaterge RAM®, and Olive oil.

Another embodiment comprises cyclosporin A, Colatrope INC®, and Olive oil.

Another embodiment comprises cyclosporin A, Crodacid B®, and Olive oil.

Another embodiment comprises cyclosporin A, DeTrope CA-100®, and Olive oil.

Another embodiment comprises cyclosporin A, Latol MTO®, and Olive oil.

Another embodiment comprises cyclosporin A, Lumulse CC-33 K®, and Olive oil.

Another embodiment comprises cyclosporin A, Mulls 2218®, and Olive oil.

Another embodiment comprises cyclosporin A, OL-600®, and Olive oil.

Another embodiment comprises cyclosporin A, OL-800®, and Olive oil.

Another embodiment comprises cyclosporin A, R-910®, and Olive oil.

Another embodiment comprises cyclosporin A, S-210®, and Olive oil.

Another embodiment comprises cyclosporin A, Sandopan DTC Acid®, and Olive oil.

Another embodiment comprises cyclosporin A, Sandopan LS 24 N®, and Olive oil.

Another embodiment comprises cyclosporin A, Sandopan MA-18®, and Olive oil.

Another embodiment comprises cyclosporin A, an Ethoxylated Alcohol, and Olive oil.

Another embodiment comprises cyclosporin A, Adsee 799®, and Olive oil.

Another embodiment comprises cyclosporin A, Adsee 799®, and Olive oil.

Another embodiment comprises cyclosporin A, Alfonic 610-3.5®, and Olive oil.

Another embodiment comprises cyclosporin A, Alfonic 810-2®, and Olive oil.

Another embodiment comprises cyclosporin A, Alfonic 810-6®, and Olive oil.

Another embodiment comprises cyclosporin A, Alfonic 1012-3®, and Olive oil.

Another embodiment comprises cyclosporin A, Alfonic 1012-5®, and Olive oil.

Another embodiment comprises cyclosporin A, Alfonic 1216CO-1.5®, and Olive oil.

Another embodiment comprises cyclosporin A, Alfonic 1216CO-7®, and Olive oil.

Another embodiment comprises cyclosporin A, Alfonic 1412-3®, and Olive oil.

Another embodiment comprises cyclosporin A, Alfonic 1412-7®, and Olive oil.

Another embodiment comprises cyclosporin A, Arlasolve 200®, and Olive oil.

Another embodiment comprises cyclosporin A, Arlasolve 200 Liquid®, and Olive oil.

Another embodiment comprises cyclosporin A, Armix 180-C®, and Olive oil.

Another embodiment comprises cyclosporin A, Armix 183®, and Olive oil.

Another embodiment comprises cyclosporin A, Armul 2404®, and Olive oil.

Another embodiment comprises cyclosporin A, Armul 2404®, and Olive oil.

Another embodiment comprises cyclosporin A, Atlas EMJ-C®, and Olive oil.

Another embodiment comprises cyclosporin A, Atlas G-2109®, and Olive oil.

Another embodiment comprises cyclosporin A, Atlas G-3886®, and Olive oil.

Another embodiment comprises cyclosporin A, Atlas G-3890®, and Olive oil.

Another embodiment comprises cyclosporin A, Bio Soft E-200®, and Olive oil.

Another embodiment comprises cyclosporin A, Bio Soft E-300®, and Olive oil.

Another embodiment comprises cyclosporin A, Bio Soft E-400®, and Olive oil.

Another embodiment comprises cyclosporin A, Bio Soft EN 600®, and Olive oil.

Another embodiment comprises cyclosporin A, Bio Soft TD-400®, and Olive oil.

Another embodiment comprises cyclosporin A, Bio Soft TD-630®, and Olive oil.

Another embodiment comprises cyclosporin A, Brij 30®, and Olive oil.

Another embodiment comprises cyclosporin A, Brij 52®, and Olive oil.

Another embodiment comprises cyclosporin A, Brij 56®, and Olive oil.

Another embodiment comprises cyclosporin A, Brij 58®, and Olive oil.

Another embodiment comprises cyclosporin A, Brij 72®, and Olive oil.

Another embodiment comprises cyclosporin A, Brij 76®, and Olive oil.

Another embodiment comprises cyclosporin A, Brij 78®, and Olive oil.

Another embodiment comprises cyclosporin A, Brij 93®, and Olive oil.

Another embodiment comprises cyclosporin A, Brij 97®, and Olive oil.

Another embodiment comprises cyclosporin A, Brij 98®, and Olive oil.

Another embodiment comprises cyclosporin A, Brij 700®, and Olive oil.

Another embodiment comprises cyclosporin A, Brij 700 S®, and Olive oil.

Another embodiment comprises cyclosporin A, Brij 721®, and Olive oil.

Another embodiment comprises cyclosporin A, Brij 721 S®, and Olive oil.

Another embodiment comprises cyclosporin A, Burcoterge CDG®, and Olive oil.

Another embodiment comprises cyclosporin A, Canasol AT 600®, and Olive oil.

Another embodiment comprises cyclosporin A, Canasol AT 800®, and Olive oil.

Another embodiment comprises cyclosporin A, Canasol AT 1200®, and Olive oil.

Another embodiment comprises cyclosporin A, Canasol BJ 35®, and Olive oil.

Another embodiment comprises cyclosporin A, Canasol BJ 36®, and Olive oil.

Another embodiment comprises cyclosporin A, Canasol BJ 52®, and Olive oil.

Another embodiment comprises cyclosporin A, Canasol BJ 58®, and Olive oil.

Another embodiment comprises cyclosporin A, Canasol BJ 72®, and Olive oil.

Another embodiment comprises cyclosporin A, Canasol BJ 78®, and Olive oil.

Another embodiment comprises cyclosporin A, Canasol BJ 98®, and Olive oil.

Another embodiment comprises cyclosporin A, Canasol BJ 307®, and Olive oil.

Another embodiment comprises cyclosporin A, Cerfak 1400®, and Olive oil.

Another embodiment comprises cyclosporin A, Cetomacrogol 1000 BP®, and Olive oil.

Another embodiment comprises cyclosporin A, Chemonic C-2®, and Olive oil.

Another embodiment comprises cyclosporin A, Chemonic C-10®, and Olive oil.

Another embodiment comprises cyclosporin A, Chemonic C-20®, and Olive oil.

Another embodiment comprises cyclosporin A, Chemonic CT-12®, and Olive oil.

Another embodiment comprises cyclosporin A, Chemonic CT-20®, and Olive oil.

Another embodiment comprises cyclosporin A, Chemonic CT-30®, and Olive oil.

Another embodiment comprises cyclosporin A, Chemonic CT-55®, and Olive oil.

Another embodiment comprises cyclosporin A, Chemonic G-7®, and Olive oil.

Another embodiment comprises cyclosporin A, Chemonic G-26®, and Olive oil.

Another embodiment comprises cyclosporin A, Chemonic L-4®, and Olive oil.

Another embodiment comprises cyclosporin A, Chemonic L-7®, and Olive oil.

Another embodiment comprises cyclosporin A, Chemonic L-12®, and Olive oil.

Another embodiment comprises cyclosporin A, Chemonic L-23®, and Olive oil.

Another embodiment comprises cyclosporin A, Chemonic O-2®, and Olive oil.

Another embodiment comprises cyclosporin A, Chemonic O-5®, and Olive oil.

Another embodiment comprises cyclosporin A, Chemonic O-10®, and Olive oil.

Another embodiment comprises cyclosporin A, Chemonic O-20®, and Olive oil.

Another embodiment comprises cyclosporin A, Chemonic S-2®, and Olive oil.

Another embodiment comprises cyclosporin A, Chemonic S-10®, and Olive oil.

Another embodiment comprises cyclosporin A, Chemonic S-20®, and Olive oil.

Another embodiment comprises cyclosporin A, Colamulse FE®, and Olive oil.

Another embodiment comprises cyclosporin A, Cremophor A 20®, and Olive oil.

Another embodiment comprises cyclosporin A, Cremophor SA 2®, and Olive oil.

Another embodiment comprises cyclosporin A, Dehydol 100®, and Olive oil.

Another embodiment comprises cyclosporin A, Dehydol O-4®, and Olive oil.

Another embodiment comprises cyclosporin A, Delonic C-18®, and Olive oil.

Another embodiment comprises cyclosporin A, DeSonic 6T®, and Olive oil.

Another embodiment comprises cyclosporin A, DeSonic 9D®, and Olive oil.

Another embodiment comprises cyclosporin A, DeSonic 9T®, and Olive oil.

Another embodiment comprises cyclosporin A, DeSonic 12D®, and Olive oil.

Another embodiment comprises cyclosporin A, DeSonic 12T®, and Olive oil.

Another embodiment comprises cyclosporin A, DeSonic 15T®, and Olive oil.

Another embodiment comprises cyclosporin A, DeSonic TDA-9®, and Olive oil.

Another embodiment comprises cyclosporin A, DeThox GLG-7®, and Olive oil.

Another embodiment comprises cyclosporin A, DeThox GLG-26®, and Olive oil.

Another embodiment comprises cyclosporin A, DeThox LA-4®, and Olive oil.

Another embodiment comprises cyclosporin A, DeThox LA-23®, and Olive oil.

Another embodiment comprises cyclosporin A, DeThox SA-80®, and Olive oil.

Another embodiment comprises cyclosporin A, Disponil O5®, and Olive oil.

Another embodiment comprises cyclosporin A, Eccoterge EO-41B®, and Olive oil.

Another embodiment comprises cyclosporin A, Empilan KA2.5/90®, and Olive oil.

Another embodiment comprises cyclosporin A, Empilan KA5/90®, and Olive oil.

Another embodiment comprises cyclosporin A, Empilan KM-20®, and Olive oil.

Another embodiment comprises cyclosporin A, Empilan KM-50®, and Olive oil.

Another embodiment comprises cyclosporin A, Empilan L-23®, and Olive oil.

Another embodiment comprises cyclosporin A, Ethylan 25-3®, and Olive oil.

Another embodiment comprises cyclosporin A, Ethylan 1204®, and Olive oil.

Another embodiment comprises cyclosporin A, Ethylan DA-4®, and Olive oil.

Another embodiment comprises cyclosporin A, Ethylan LA-230®, and Olive oil.

Another embodiment comprises cyclosporin A, Ethylan SN®, and Olive oil.

Another embodiment comprises cyclosporin A, Ethylan TD-60®, and Olive oil.

Another embodiment comprises cyclosporin A, Ethylan TD-100®, and Olive oil.

Another embodiment comprises cyclosporin A, Ethylan TD-1407®, and Olive oil.

Another embodiment comprises cyclosporin A, Eumulgin B1®, and Olive oil.

Another embodiment comprises cyclosporin A, Eumulgin B2®, and Olive oil.

Another embodiment comprises cyclosporin A, Eumulgin B3®, and Olive oil.

Another embodiment comprises cyclosporin A, Eumulgin O-10®, and Olive oil.

Another embodiment comprises cyclosporin A, Flo Mo 80/20®, and Olive oil.

Another embodiment comprises cyclosporin A, Flo Mo Low Foam®, and Olive oil.

Another embodiment comprises cyclosporin A, Forlan C-24®, and Olive oil.

Another embodiment comprises cyclosporin A, Genapol 1454®, and Olive oil.

Another embodiment comprises cyclosporin A, Genapol BA-020®, and Olive oil.

Another embodiment comprises cyclosporin A, Genapol BA-040®, and Olive oil.

Another embodiment comprises cyclosporin A, Genapol C-100®, and Olive oil.

Another embodiment comprises cyclosporin A, Genapol DA 060®, and Olive oil.

Another embodiment comprises cyclosporin A, Genapol HS 020®, and Olive oil.

Another embodiment comprises cyclosporin A, Genapol HS 200®, and Olive oil.

Another embodiment comprises cyclosporin A, Genapol ID-040®, and Olive oil.

Another embodiment comprises cyclosporin A, Genapol ID-060®, and Olive oil.

Another embodiment comprises cyclosporin A, Genapol ID-090®, and Olive oil.

Another embodiment comprises cyclosporin A, Genapol LA 010®, and Olive oil.

Another embodiment comprises cyclosporin A, Genapol LA 020®, and Olive oil.

Another embodiment comprises cyclosporin A, Genapol LA 030®, and Olive oil.

Another embodiment comprises cyclosporin A, Genapol LA 040®, and Olive oil.

Another embodiment comprises cyclosporin A, Genapol LA 050®, and Olive oil.

Another embodiment comprises cyclosporin A, Genapol LA 060®, and Olive oil.

Another embodiment comprises cyclosporin A, Genapol LA 070®, and Olive oil.

Another embodiment comprises cyclosporin A, Genapol LA 070S®, and Olive oil.

Another embodiment comprises cyclosporin A, Genapol LA 230®, and Olive oil.

Another embodiment comprises cyclosporin A, Genapol O 020®, and Olive oil.

Another embodiment comprises cyclosporin A, Genapol O 050®, and Olive oil.

Another embodiment comprises cyclosporin A, Genapol O 100®, and Olive oil.

Another embodiment comprises cyclosporin A, Genapol O 200®, and Olive oil.

Another embodiment comprises cyclosporin A, Genapol SA 030®, and Olive oil.

Another embodiment comprises cyclosporin A, Genapol SA 120®, and Olive oil.

Another embodiment comprises cyclosporin A, Genapol T-020®, and Olive oil.

Another embodiment comprises cyclosporin A, Genapol UD-030®, and Olive oil.

Another embodiment comprises cyclosporin A, Genapol UD-050®, and Olive oil.

Another embodiment comprises cyclosporin A, Genapol UD-070®, and Olive oil.

Another embodiment comprises cyclosporin A, Genapol UD-079®, and Olive oil.

Another embodiment comprises cyclosporin A, Genapol UD-080®, and Olive oil.

Another embodiment comprises cyclosporin A, Genapol UD-110®, and Olive oil.

Another embodiment comprises cyclosporin A, Genapol X 030®, and Olive oil.

Another embodiment comprises cyclosporin A, Genapol X 050®, and Olive oil.

Another embodiment comprises cyclosporin A, Genapol X 060®, and Olive oil.

Another embodiment comprises cyclosporin A, Genapol X 070®, and Olive oil.

Another embodiment comprises cyclosporin A, Genapbl X 080®, and Olive oil.

Another embodiment comprises cyclosporin A, Genapol X 100®, and Olive oil.

Another embodiment comprises cyclosporin A, Genapol X159®, and Olive oil.

Another embodiment comprises cyclosporin A, Generol 122 E5®, and Olive oil.

Another embodiment comprises cyclosporin A, Generol 122 E25®, and Olive oil.

Another embodiment comprises cyclosporin A, Hostacerin T-3®, and Olive oil.

Another embodiment comprises cyclosporin A, Iconol LF 110®, and Olive oil.

Another embodiment comprises cyclosporin A, Incropol CS-20®, and Olive oil.

Another embodiment comprises cyclosporin A, Lexemul CS-20®, and Olive oil.

Another embodiment comprises cyclosporin A, Liponic EG-1®, and Olive oil.

Another embodiment comprises cyclosporin A, Lipowax D®, and Olive oil.

Another embodiment comprises cyclosporin A, Lipowax G®, and Olive oil.

Another embodiment comprises cyclosporin A, Lipowax NI®, and Olive oil.

Another embodiment comprises cyclosporin A, Lipowax P®, and Olive oil.

Another embodiment comprises cyclosporin A, Lipowax P-31®, and Olive oil.

Another embodiment comprises cyclosporin A, Lipowax PR®, and Olive oil.

Another embodiment comprises cyclosporin A, Lumulse CS-20®, and Olive oil.

Another embodiment comprises cyclosporin A, Macol CSA-20®, and Olive oil.

Another embodiment comprises cyclosporin A, Marlox B 24/50®, and Olive oil.

Another embodiment comprises cyclosporin A, Mazawet 77®, and Olive oil.

Another embodiment comprises cyclosporin A, Norfox 1713®, and Olive oil.

Another embodiment comprises cyclosporin A, Norfox 2579®, and Olive oil.

Another embodiment comprises cyclosporin A, Norox Lo Foam®, and Olive oil.

Another embodiment comprises cyclosporin A, Promulgen D®, and Olive oil.

Another embodiment comprises cyclosporin A, Promulgen G®, and Olive oil.

Another embodiment comprises cyclosporin A, Renex 30®, and Olive oil.

Another embodiment comprises cyclosporin A, Renex 36®, and Olive oil.

Another embodiment comprises cyclosporin A, Rhodasurf A 24®, and Olive oil.

Another embodiment comprises cyclosporin A, Rhodasurf AAE-10®, and Olive oil.

Another embodiment comprises cyclosporin A, Rhodasurf BEH-25®, and Olive oil.

Another embodiment comprises cyclosporin A, Rhodasurf BEH-40®, and Olive oil.

Another embodiment comprises cyclosporin A, Rhodasurf DA 530®, and Olive oil.

Another embodiment comprises cyclosporin A, Rhodasurf DA 630®, and Olive oil.

Another embodiment comprises cyclosporin A, Rhodasurf DA 639®, and Olive oil.

Another embodiment comprises cyclosporin A, Rhodasurf LAN-23®, and Olive oil.

Another embodiment comprises cyclosporin A, Rhodasurf ON-870®, and Olive oil.

Another embodiment comprises cyclosporin A, Rhodasurf ON-877®, and Olive oil.

Another embodiment comprises cyclosporin A, Rhodasurf TB-970 FLK®, and Olive oil.

Another embodiment comprises cyclosporin A, Ritacet-20®, and Olive oil.

Another embodiment comprises cyclosporin A, Ritachol 1000®, and Olive oil.

Another embodiment comprises cyclosporin A, Ritachol 2000®, and Olive oil.

Another embodiment comprises cyclosporin A, Ritachol 5000®, and Olive oil.

Another embodiment comprises cyclosporin A, Ritox 35®, and Olive oil.

Another embodiment comprises cyclosporin A, Surlonic DA-4®, and Olive oil.

Another embodiment comprises cyclosporin A, Surfonic DA-6®, and Olive oil.

Another embodiment comprises cyclosporin A, Surfonic L46-7®, and Olive oil.

Another embodiment comprises cyclosporin A, Surdonic POA®, and Olive oil.

Another embodiment comprises cyclosporin A, Synthrapol KB®, and Olive oil.

Another embodiment comprises cyclosporin A, Teginacid®, and Olive oil.

Another embodiment comprises cyclosporin A, Teginacid C®, and Olive oil.

Another embodiment comprises cyclosporin A, Tegotens EC 11®, and Olive oil.

Another embodiment comprises cyclosporin A, Tinegal NA®, and Olive oil.

Another embodiment comprises cyclosporin A, Tomadol 400®, and Olive oil.

Another embodiment comprises cyclosporin A, Tomadol 600®, and Olive oil.

Another embodiment comprises cyclosporin A, Tomadol 900®, and Olive oil.

Another embodiment comprises cyclosporin A, Uniperol O®, and Olive oil.

Another embodiment comprises cyclosporin A, Witconol SN Series®, and Olive oil.

Another embodiment comprises cyclosporin A, an Ethoxylated Alkylphenol, and Olive oil.

Another embodiment comprises cyclosporin A, Antarox LF-222®, and Olive oil.

Another embodiment comprises cyclosporin A, Atlox 775®, and Olive oil.

Another embodiment comprises cyclosporin A, Caloxylate N-9®, and Olive oil.

Another embodiment comprises cyclosporin A, Canasol NF-1000®, and Olive oil.

Another embodiment comprises cyclosporin A, Canasol NF-3000®, and Olive oil.

Another embodiment comprises cyclosporin A, Canasol NF-3070®, and Olive oil.

Another embodiment comprises cyclosporin A, Canasol OF 1670®, and Olive oil.

Another embodiment comprises cyclosporin A, Canasol OF 2570®, and Olive oil.

Another embodiment comprises cyclosporin A, Canasol OF 4070®, and Olive oil.

Another embodiment comprises cyclosporin A, Chemax DNP-8®, and Olive oil.

Another embodiment comprises cyclosporin A, Chemax DNP-18®, and Olive oil.

Another embodiment comprises cyclosporin A, Chemax DNP-150/50®, and Olive oil.

Another embodiment comprises cyclosporin A, DeSonic 1.5N®, and Olive oil.

Another embodiment comprises cyclosporin A, DeSonic 4N®, and Olive oil.

Another embodiment comprises cyclosporin A, DeSonic 5N®, and Olive oil.

Another embodiment comprises cyclosporin A, DeSonic 6D®, and Olive oil.

Another embodiment comprises cyclosporin A, DeSonic 6N®, and Olive oil.

Another embodiment comprises cyclosporin A, DeSonic 7N®, and Olive oil.

Another embodiment comprises cyclosporin A, DeSonic 9N®, and Olive oil.

Another embodiment comprises cyclosporin A, DeSonic 10D®, and Olive oil.

Another embodiment comprises cyclosporin A, DeSonic 11N®, and Olive oil.

Another embodiment comprises cyclosporin A, DeSonic 12N®, and Olive oil.

Another embodiment comprises cyclosporin A, DeSonic 15N®, and Olive oil.

Another embodiment comprises cyclosporin A, DeSonic 20N®, and Olive oil.

Another embodiment comprises cyclosporin A, Eccoscour RC®, and Olive oil.

Another embodiment comprises cyclosporin A, Eccoterge EO-100®, and Olive oil.

Another embodiment comprises cyclosporin A, Emulsifier 632/90%®, and Olive oil.

Another embodiment comprises cyclosporin A, Geronol AG-821®, and Olive oil.

Another embodiment comprises cyclosporin A, Gradonic N-95®, and Olive oil.

Another embodiment comprises cyclosporin A, Hetoxide NP-4®, and Olive oil.

Another embodiment comprises cyclosporin A, Hetoxide NP-30®, and Olive oil.

Another embodiment comprises cyclosporin A, Hostapal N-100®, and Olive oil.

Another embodiment comprises cyclosporin A, Hostapal N-110®, and Olive oil.

Another embodiment comprises cyclosporin A, Igepal CTA-639W®, and Olive oil.

Another embodiment comprises cyclosporin A, Igepal DAP-9®, and Olive oil.

Another embodiment comprises cyclosporin A, Igepal OD-410®, and Olive oil.

Another embodiment comprises cyclosporin A, Igepal SS-837®, and Olive oil.

Another embodiment comprises cyclosporin A, Lipocol NP-9 USP®, and Olive oil.

Another embodiment comprises cyclosporin A, Macol DNP-10®, and Olive oil.

Another embodiment comprises cyclosporin A, Marlophen NP 5®, and Olive oil.

Another embodiment comprises cyclosporin A, Marlophen P 1®, and Olive oil.

Another embodiment comprises cyclosporin A, Surfonic NB®, and Olive oil.

Another embodiment comprises cyclosporin A, Surfonic OPB-307®, and Olive oil.

Another embodiment comprises cyclosporin A, Surfonic OPB-407®, and Olive oil.

Another embodiment comprises cyclosporin A, Syn Fac 334®, and Olive oil.

Another embodiment comprises cyclosporin A, Syn Fac 8216®, and Olive oil.

Another embodiment comprises cyclosporin A, Triton N-57®, and Olive oil.

Another embodiment comprises cyclosporin A, Trycol 6956®, and Olive oil.

Another embodiment comprises cyclosporin A, Trycol 6961®, and Olive oil.

Another embodiment comprises cyclosporin A, Trycol 6964®, and Olive oil.

Another embodiment comprises cyclosporin A, Trycol 6969®, and Olive oil.

Another embodiment comprises cyclosporin A, Trycol 6974®, and Olive oil.

Another embodiment comprises cyclosporin A, Witbreak DRB-127®, and Olive oil.

Another embodiment comprises cyclosporin A, Witbreak DRB-127®, and Olive oil.

Another embodiment comprises cyclosporin A, Witconol NP Series®, and Olive oil.

Another embodiment comprises cyclosporin A, an Ethoxylated Aryl Phenol, and Olive oil.

Another embodiment comprises cyclosporin A, Soprophor BSU®, and Olive oil.

Another embodiment comprises cyclosporin A, Soprophor CY/8®, and Olive oil.

Another embodiment comprises cyclosporin A, Soprophor S/25®, and Olive oil.

Another embodiment comprises cyclosporin A, Witconol NIO®, and Olive oil.

Another embodiment comprises cyclosporin A, Witconol NIW®, and Olive oil.

Another embodiment comprises cyclosporin A, Witconol S-100®, and Olive oil.

Another embodiment comprises cyclosporin A, an Ethoxylated Fatty Acid, and Olive oil.

Another embodiment comprises cyclosporin A, Aldo PGHMS®, and Olive oil.

Another embodiment comprises cyclosporin A, Alkamuls TO-15/HR®, and Olive oil.

Another embodiment comprises cyclosporin A, Armotan AL-69-66®, and Olive oil.

Another embodiment comprises cyclosporin A, Cerasynt 840®, and Olive oil.

Another embodiment comprises cyclosporin A, Cerasynt 945®, and Olive oil.

Another embodiment comprises cyclosporin A, Crystal Inhibitor No. 5®, and Olive oil.

Another embodiment comprises cyclosporin A, DeThox Acid L-9®, and Olive oil.

Another embodiment comprises cyclosporin A, DeThox Acid S-8®, and Olive oil.

Another embodiment comprises cyclosporin A, Ethofat 242/25®, and Olive oil.

Another embodiment comprises cyclosporin A, Hydropalat 65®, and Olive oil.

Another embodiment comprises cyclosporin A, Lipo EGMS®, and Olive oil.

Another embodiment comprises cyclosporin A, Lipopeg 2 DL®, and Olive oil.

Another embodiment comprises cyclosporin A, Lipopeg 4 DL®, and Olive oil.

Another embodiment comprises cyclosporin A, Lipopeg 4-L®, and Olive oil.

Another embodiment comprises cyclosporin A, Lipopeg 39-S®, and Olive oil.

Another embodiment comprises cyclosporin A, Lipopeg 4-S®, and Olive oil.

Another embodiment comprises cyclosporin A, Lipopeg 10-S®, and Olive oil.

Another embodiment comprises cyclosporin A, Lipopeg 100-S®, and Olive oil.

Another embodiment comprises cyclosporin A, Lipopeg 6000 DS®, and Olive oil.

Another embodiment comprises cyclosporin A, Lumulse 40-L®, and Olive oil.

Another embodiment comprises cyclosporin A, Lumulse 40-S®, and Olive oil.

Another embodiment comprises cyclosporin A, Lumulse 42-L®, and Olive oil.

Another embodiment comprises cyclosporin A, Lumulse 42-S®, and Olive oil.

Another embodiment comprises cyclosporin A, Lumulse 100-S®, and Olive oil.

Another embodiment comprises cyclosporin A, Lumulse 602-S®, and Olive oil.

Another embodiment comprises cyclosporin A, Magrabar PGE-20-0®, and Olive oil.

Another embodiment comprises cyclosporin A, Magrabar PGE-20L®, and Olive oil.

Another embodiment comprises cyclosporin A, Magrabar PGE-20T®, and Olive oil.

Another embodiment comprises cyclosporin A, Magrabar PGE-22-0®, and Olive oil.

Another embodiment comprises cyclosporin A, Magrabar PGE-22L®, and Olive oil.

Another embodiment comprises cyclosporin A, Magrabar PGE-22T®, and Olive oil.

Another embodiment comprises cyclosporin A, Magrabar PGE-40-0®, and Olive oil.

Another embodiment comprises cyclosporin A, Magrabar PGE-40L®, and Olive oil.

Another embodiment comprises cyclosporin A, Magrabar PGE-40T®, and Olive oil.

Another embodiment comprises cyclosporin A, Magrabar PGE-42-0®, and Olive oil.

Another embodiment comprises cyclosporin A, Magrabar PGE-42L®, and Olive oil.

Another embodiment comprises cyclosporin A, Magrabar PGE-42T®, and Olive oil.

Another embodiment comprises cyclosporin A, Magrabar PGE-60-0®, and Olive oil.

Another embodiment comprises cyclosporin A, Magrabar PGE-60L®, and Olive oil.

Another embodiment comprises cyclosporin A, Magrabar PGE-60T®, and Olive oil.

Another embodiment comprises cyclosporin A, Magrabar PGE-62-0®, and Olive oil.

Another embodiment comprises cyclosporin A, Magrabar PGE-62L®, and Olive oil.

Another embodiment comprises cyclosporin A, Magrabar PGE-62T®, and Olive oil.

Another embodiment comprises cyclosporin A, Mapeg S-40K®, and Olive oil.

Another embodiment comprises cyclosporin A, Marlowet OTS®, and Olive oil.

Another embodiment comprises cyclosporin A, Naturechem PGR®, and Olive oil.

Another embodiment comprises cyclosporin A, PG No. 4®, and Olive oil.

Another embodiment comprises cyclosporin A, Renex 20®, and Olive oil.

Another embodiment comprises cyclosporin A, Ritox 52®, and Olive oil.

Another embodiment comprises cyclosporin A, Ritox 53®, and Olive oil.

Another embodiment comprises cyclosporin A, Ritox 59®, and Olive oil.

Another embodiment comprises cyclosporin A, Surfax 8916/A®, and Olive oil.

Another embodiment comprises cyclosporin A, Tego Acid S 40 P®, and Olive oil.

Another embodiment comprises cyclosporin A, Tego Acid S 100 P®, and Olive oil.

Another embodiment comprises cyclosporin A, Tween 20®, and Olive oil.

Another embodiment comprises cyclosporin A, Volpo 131®, and Olive oil.

Another embodiment comprises cyclosporin A, an Ethoxylated Fatty Ester or Oil, and Olive oil.

Another embodiment comprises cyclosporin A, Acconon 6-C10®, and Olive oil.

Another embodiment comprises cyclosporin A, Acconon CC-6®, and Olive oil.

Another embodiment comprises cyclosporin A, Acconon CO-7®, and Olive oil.

Another embodiment comprises cyclosporin A, Aldosperse 40/60 FG®, and Olive oil.

Another embodiment comprises cyclosporin A, Aldosperse ML-23®, and Olive oil.

Another embodiment comprises cyclosporin A, Aldosperse MS-20 FG®, and Olive oil.

Another embodiment comprises cyclosporin A, Alkamuls EL-620®, and Olive oil.

Another embodiment comprises cyclosporin A, Alkamuls EL-719®, and Olive oil.

Another embodiment comprises cyclosporin A, Alkamuls EL-985®, and Olive oil.

Another embodiment comprises cyclosporin A, Arlatone G®, and Olive oil.

Another embodiment comprises cyclosporin A, Arlatone T®, and Olive oil.

Another embodiment comprises cyclosporin A, Atlas G-1045A®, and Olive oil.

Another embodiment comprises cyclosporin A, Atlas G-1086®, and Olive oil.

Another embodiment comprises cyclosporin A, Atlas G-1087®, and Olive oil.

Another embodiment comprises cyclosporin A, Atlas G-1089®, and Olive oil.

Another embodiment comprises cyclosporin A, Atlas G-1096®, and Olive oil.

Another embodiment comprises cyclosporin A, Atlas G-1292®, and Olive oil.

Another embodiment comprises cyclosporin A, Atlas G-1293®, and Olive oil.

Another embodiment comprises cyclosporin A, Atlas G-1300®, and Olive oil.

Another embodiment comprises cyclosporin A, Atlas G-7076®, and Olive oil.

Another embodiment comprises cyclosporin A, Capmul EMG®, and Olive oil.

Another embodiment comprises cyclosporin A, Chemonic CO-40®, and Olive oil.

Another embodiment comprises cyclosporin A, Chemonic LI-3®, and Olive oil.

Another embodiment comprises cyclosporin A, Chemonic LI-7®, and Olive oil.

Another embodiment comprises cyclosporin A, Cirrasol GM®, and Olive oil.

Another embodiment comprises cyclosporin A, Cremophor CO 40®, and Olive oil.

Another embodiment comprises cyclosporin A, Cremophor CO 410®, and Olive oil.

Another embodiment comprises cyclosporin A, Cremophor EL®, and Olive oil.

Another embodiment comprises cyclosporin A, Cremophor GC7®, and Olive oil.

Another embodiment comprises cyclosporin A, Cremophor RH-40®, and Olive oil.

Another embodiment comprises cyclosporin A, Crovol A-40®, and Olive oil.

Another embodiment comprises cyclosporin A, Crovol A-70®, and Olive oil.

Another embodiment comprises cyclosporin A, Crovol M-70®, and Olive oil.

Another embodiment comprises cyclosporin A, Crovol PK-70®, and Olive oil.

Another embodiment comprises cyclosporin A, Cutina E-24®, and Olive oil.

Another embodiment comprises cyclosporin A, Dacospin 12-R®, and Olive oil.

Another embodiment comprises cyclosporin A, Dehymuls HRE-7®, and Olive oil.

Another embodiment comprises cyclosporin A, DeSonic 30C®, and Olive oil.

Another embodiment comprises cyclosporin A, DeSonic 36C®, and Olive oil.

Another embodiment comprises cyclosporin A, DeSonic 40C®, and Olive oil.

Another embodiment comprises cyclosporin A, Durfax 60®, and Olive oil.

Another embodiment comprises cyclosporin A, Durfax 65®, and Olive oil.

Another embodiment comprises cyclosporin A, Durfax 80®, and Olive oil.

Another embodiment comprises cyclosporin A, Durfax EOM®, and Olive oil.

Another embodiment comprises cyclosporin A, Eccoterge NF-2®, and Olive oil.

Another embodiment comprises cyclosporin A, Emulpon CO-360®, and Olive oil.

Another embodiment comprises cyclosporin A, Emulpon CO-550®, and Olive oil.

Another embodiment comprises cyclosporin A, Emulsogen EL®, and Olive oil.

Another embodiment comprises cyclosporin A, Emulsogen HCO 040®, and Olive oil.

Another embodiment comprises cyclosporin A, Emulsogen HCO 060®, and Olive oil.

Another embodiment comprises cyclosporin A, Emulsynt 1055®, and Olive oil.

Another embodiment comprises cyclosporin A, Ethox 3095®, and Olive oil.

Another embodiment comprises cyclosporin A, Eumulgin RO-40®, and Olive oil.

Another embodiment comprises cyclosporin A, Genapol G-260®, and Olive oil.

Another embodiment comprises cyclosporin A, Glycosperse L-20®, and Olive oil.

Another embodiment comprises cyclosporin A, Glycosperse O-5®, and Olive oil.

Another embodiment comprises cyclosporin A, Glycosperse O-20®, and Olive oil.

Another embodiment comprises cyclosporin A, Glycosperse O-20 FG®, and Olive oil.

Another embodiment comprises cyclosporin A, Glycosperse S-20®, and Olive oil.

Another embodiment comprises cyclosporin A, Glycosperse S-20 FG®, and Olive oil.

Another embodiment comprises cyclosporin A, Glycosperse TS-20®, and Olive oil.

Another embodiment comprises cyclosporin A, Glycosperse TS-20 FG®, and Olive oil.

Another embodiment comprises cyclosporin A, Hetan SL®, and Olive oil.

Another embodiment comprises cyclosporin A, Hetan SO®, and Olive oil.

Another embodiment comprises cyclosporin A, Hetan SS®, and Olive oil.

Another embodiment comprises cyclosporin A, Hetoxide C-2®, and Olive oil.

Another embodiment comprises cyclosporin A, Hetoxide C-9®, and Olive oil.

Another embodiment comprises cyclosporin A, Hetoxide C-15®, and Olive oil.

Another embodiment comprises cyclosporin A, Hetoxide C-25®, and Olive oil.

Another embodiment comprises cyclosporin A, Hetoxide C-40®, and Olive oil.

Another embodiment comprises cyclosporin A, Hetoxide C-200®, and Olive oil.

Another embodiment comprises cyclosporin A, Hetoxide C-200-50%®, and Olive oil.

Another embodiment comprises cyclosporin A, Hetoxide GC-30®, and Olive oil.

Another embodiment comprises cyclosporin A, Hetoxide HC-60®, and Olive oil.

Another embodiment comprises cyclosporin A, Ice No. 2®, and Olive oil.

Another embodiment comprises cyclosporin A, Incrocas 30/40®, and Olive oil.

Another embodiment comprises cyclosporin A, Lexol EC®, and Olive oil.

Another embodiment comprises cyclosporin A, Lexol EO®, and Olive oil.

Another embodiment comprises cyclosporin A, Lipocol HCO-40®, and Olive oil.

Another embodiment comprises cyclosporin A, Lipocol HCO-60®, and Olive oil.

Another embodiment comprises cyclosporin A, Lipocol O-3 Special®, and Olive oil.

Another embodiment comprises cyclosporin A, Lipopeg 2-L®, and Olive oil.

Another embodiment comprises cyclosporin A, Lipopeg 4-DO®, and Olive oil.

Another embodiment comprises cyclosporin A, Lipopeg 4-DS®, and Olive oil.

Another embodiment comprises cyclosporin A, Lipovol GTB®, and Olive oil.

Another embodiment comprises cyclosporin A, Lonzest SML-20®, and Olive oil.

Another embodiment comprises cyclosporin A, Lonzest SMO-20®, and Olive oil.

Another embodiment comprises cyclosporin A, Lonzest SMS-20®, and Olive oil.

Another embodiment comprises cyclosporin A, Lonzest STO-20®, and Olive oil.

Another embodiment comprises cyclosporin A, Lonzest STS-20®, and Olive oil.

Another embodiment comprises cyclosporin A, Lumulse GR-40®, and Olive oil.

Another embodiment comprises cyclosporin A, Lumulse GRH-40®, and Olive oil.

Another embodiment comprises cyclosporin A, Lumulse POE (7) GML®, and Olive oil.

Another embodiment comprises cyclosporin A, Lumulse POE (12) Glyc®, and Olive oil.

Another embodiment comprises cyclosporin A, Lumulse POE (40) MS KP®, and Olive oil.

Another embodiment comprises cyclosporin A, Marlowet 4750®, and Olive oil.

Another embodiment comprises cyclosporin A, Marlowet LVS®, and Olive oil.

Another embodiment comprises cyclosporin A, Marlowet R 11®, and Olive oil.

Another embodiment comprises cyclosporin A, Marlowet R 40®, and Olive oil.

Another embodiment comprises cyclosporin A, Mazol 80 MGK®, and Olive oil.

Another embodiment comprises cyclosporin A, Nonionic Emulsifier T-9®, and Olive oil.

Another embodiment comprises cyclosporin A, Oronal LCG®, and Olive oil.

Another embodiment comprises cyclosporin A, Polyderm PPI-CO-200®, and Olive oil.

Another embodiment comprises cyclosporin A, Polyderm PPI-CO-40®, and Olive oil.

Another embodiment comprises cyclosporin A, Rewoderm LI 520-70®, and Olive oil.

Another embodiment comprises cyclosporin A, Ritapeg 150 DS®, and Olive oil.

Another embodiment comprises cyclosporin A, Softigen 767®, and Olive oil.

Another embodiment comprises cyclosporin A, Surfactol 318®, and Olive oil.

Another embodiment comprises cyclosporin A, Surfactol 365®, and Olive oil.

Another embodiment comprises cyclosporin A, Syn Lube 107®, and Olive oil.

Another embodiment comprises cyclosporin A, Syn Lube 728®, and Olive oil.

Another embodiment comprises cyclosporin A, Syn Lube 1632H®, and Olive oil.

Another embodiment comprises cyclosporin A, Syn Lube 6277-A®, and Olive oil.

Another embodiment comprises cyclosporin A, T-Det C-20®, and Olive oil.

Another embodiment comprises cyclosporin A, T-Det C-40®, and Olive oil.

Another embodiment comprises cyclosporin A, Tally 100 Plus®, and Olive oil.

Another embodiment comprises cyclosporin A, Uniperol EL®, and Olive oil.

Another embodiment comprises cyclosporin A, a Fatty Ester, and Olive oil.

Another embodiment comprises cyclosporin A, Actralube-Syn 147®, and Olive oil.

Another embodiment comprises cyclosporin A, Atlas G-1556®, and Olive oil.

Another embodiment comprises cyclosporin A, Atlas G-1564®, and Olive oil.

Another embodiment comprises cyclosporin A, Atlasol Base Oil S®, and Olive oil.

Another embodiment comprises cyclosporin A, Base ML®, and Olive oil.

Another embodiment comprises cyclosporin A, Base MT®, and Olive oil.

Another embodiment comprises cyclosporin A, Cerasynt 303®, and Olive oil.

Another embodiment comprises cyclosporin A, Dermol 1012®, and Olive oil.

Another embodiment comprises cyclosporin A, Kemester 4000®, and Olive oil.

Another embodiment comprises cyclosporin A, Lactipol S®, and Olive oil.

Another embodiment comprises cyclosporin A, Magrabar PGO®, and Olive oil.

Another embodiment comprises cyclosporin A, Mayco Base BFO®, and Olive oil.

Another embodiment comprises cyclosporin A, Methyl Linoleate®, and Olive oil.

Another embodiment comprises cyclosporin A, Pationic 122A®, and Olive oil.

Another embodiment comprises cyclosporin A, Pationic 138C®, and Olive oil.

Another embodiment comprises cyclosporin A, Pationic CSL®, and Olive oil.

Another embodiment comprises cyclosporin A, Pationic ISL®, and Olive oil.

Another embodiment comprises cyclosporin A, Pationic SBL®, and Olive oil.

Another embodiment comprises cyclosporin A, Pationic SSL®, and Olive oil.

Another embodiment comprises cyclosporin A, Ritasol®, and Olive oil.

Another embodiment comprises cyclosporin A, Tego Alkanol CS 20®, and Olive oil.

Another embodiment comprises cyclosporin A, Tego Alkanol L23 P®, and Olive oil.

Another embodiment comprises cyclosporin A, Tego Alkanol S2®, and Olive oil.

Another embodiment comprises cyclosporin A, Tego Alkanol S20 P®, and Olive oil.

Another embodiment comprises cyclosporin A, Triemulsifier 600 MS®, and Olive oil.

Another embodiment comprises cyclosporin A, a Glycerol Esters, and Olive oil.

Another embodiment comprises cyclosporin A, Agro #9 Wint SBO®, and Olive oil.

Another embodiment comprises cyclosporin A, Ahcovel Base 700®, and Olive oil.

Another embodiment comprises cyclosporin A, Aldo HMS FG®, and Olive oil.

Another embodiment comprises cyclosporin A, Aldo MLD®, and Olive oil.

Another embodiment comprises cyclosporin A, Aldo MLD FG®, and Olive oil.

Another embodiment comprises cyclosporin A, Aldo MO FG®, and Olive oil.

Another embodiment comprises cyclosporin A, Aldo MS®, and Olive oil.

Another embodiment comprises cyclosporin A, Aldo MS FG®, and Olive oil.

Another embodiment comprises cyclosporin A, Aldo MS LG FG®, and Olive oil.

Another embodiment comprises cyclosporin A, Aldo MSD®, and Olive oil.

Another embodiment comprises cyclosporin A, Aldo MSD FG®, and Olive oil.

Another embodiment comprises cyclosporin A, Aldosperse O-20 FG®, and Olive oil.

Another embodiment comprises cyclosporin A, Aldosperse TS-20 FG®, and Olive oil.

Another embodiment comprises cyclosporin A, Aldosperse TS-40 FG®, and Olive oil.

Another embodiment comprises cyclosporin A, Arlacel 165®, and Olive oil.

Another embodiment comprises cyclosporin A, Arlacel 186®, and Olive oil.

Another embodiment comprises cyclosporin A, Capmul GMO®, and Olive oil.

Another embodiment comprises cyclosporin A, Capmul GMS®, and Olive oil.

Another embodiment comprises cyclosporin A, Caprol 3GO®, and Olive oil.

Another embodiment comprises cyclosporin A, Caprol 3GVS®, and Olive oil.

Another embodiment comprises cyclosporin A, Caprol 6G2S®, and Olive oil.

Another embodiment comprises cyclosporin A, Caprol 10G40®, and Olive oil.

Another embodiment comprises cyclosporin A, Caprol 10G100®, and Olive oil.

Another embodiment comprises cyclosporin A, Caprol ET®, and Olive oil.

Another embodiment comprises cyclosporin A, Caprol PGE860®, and Olive oil.

Another embodiment comprises cyclosporin A, Cerasynt 945®, and Olive oil.

Another embodiment comprises cyclosporin A, Cerasynt GMS®, and Olive oil.

Another embodiment comprises cyclosporin A, Cerasynt Q®, and Olive oil.

Another embodiment comprises cyclosporin A, Cerasynt SD®, and Olive oil.

Another embodiment comprises cyclosporin A, Cerasynt WM®, and Olive oil.

Another embodiment comprises cyclosporin A, Chemsperse 14®, and Olive oil.

Another embodiment comprises cyclosporin A, Cremophor GO-32®, and Olive oil.

Another embodiment comprises cyclosporin A, Cremophor GS11®, and Olive oil.

Another embodiment comprises cyclosporin A, Cremophor GS-32®, and Olive oil.

Another embodiment comprises cyclosporin A, Cutina KD-16®, and Olive oil.

Another embodiment comprises cyclosporin A, Dehymuls PGPH®, and Olive oil.

Another embodiment comprises cyclosporin A, Dermol DGDIS®, and Olive oil.

Another embodiment comprises cyclosporin A, Dermol DGMIS®, and Olive oil.

Another embodiment comprises cyclosporin A, Dermol G-76®, and Olive oil.

Another embodiment comprises cyclosporin A, Dermol G-7DI®, and Olive oil.

Another embodiment comprises cyclosporin A, Dermol NGDI®, and Olive oil.

Another embodiment comprises cyclosporin A, Dermolan GLH®, and Olive oil.

Another embodiment comprises cyclosporin A, Drewmulse GMO®, and Olive oil.

Another embodiment comprises cyclosporin A, Drewpol 3-5-M®, and Olive oil.

Another embodiment comprises cyclosporin A, Durlac 100 W®, and Olive oil.

Another embodiment comprises cyclosporin A, Dur-Lo®, and Olive oil.

Another embodiment comprises cyclosporin A, Dynasan 118®, and Olive oil.

Another embodiment comprises cyclosporin A, EC-25®, and Olive oil.

Another embodiment comprises cyclosporin A, EM 40®, and Olive oil.

Another embodiment comprises cyclosporin A, Emerest 2400®, and Olive oil.

Another embodiment comprises cyclosporin A, Emerest 2452®, and Olive oil.

Another embodiment comprises cyclosporin A, Empilan G-26®, and Olive oil.

Another embodiment comprises cyclosporin A, Genapol TSM®, and Olive oil.

Another embodiment comprises cyclosporin A, Hostacerin DGI®, and Olive oil.

Another embodiment comprises cyclosporin A, Hostacerin DGL®, and Olive oil.

Another embodiment comprises cyclosporin A, Hostacerin DGMS®, and Olive oil.

Another embodiment comprises cyclosporin A, Hostacerin DGSB®, and Olive oil.

Another embodiment comprises cyclosporin A, Ice No. 2®, and Olive oil.

Another embodiment comprises cyclosporin A, Imwitor 742®, and Olive oil.

Another embodiment comprises cyclosporin A, Imwitor 780 K®, and Olive oil.

Another embodiment comprises cyclosporin A, Imwitor 960 Flakes®, and Olive oil.

Another embodiment comprises cyclosporin A, Isolan GI 34®, and Olive oil.

Another embodiment comprises cyclosporin A, Isolan GO 33®, and Olive oil.

Another embodiment comprises cyclosporin A, Kemester 1000®, and Olive oil.

Another embodiment comprises cyclosporin A, Kemester 2000®, and Olive oil.

Another embodiment comprises cyclosporin A, Kemester 2000®, and Olive oil.

Another embodiment comprises cyclosporin A, Kemester 6000SE®, and Olive oil.

Another embodiment comprises cyclosporin A, Lamecreme DGE 18®, and Olive oil.

Another embodiment comprises cyclosporin A, Lexemul 515®, and Olive oil.

Another embodiment comprises cyclosporin A, Lexemul 561®, and Olive oil.

Another embodiment comprises cyclosporin A, Lexemul AR®, and Olive oil.

Another embodiment comprises cyclosporin A, Lexemul AS®, and Olive oil.

Another embodiment comprises cyclosporin A, Lexemul GDL®, and Olive oil.

Another embodiment comprises cyclosporin A, Lexemul T®, and Olive oil.

Another embodiment comprises cyclosporin A, Lipomulse 165®, and Olive oil.

Another embodiment comprises cyclosporin A, Lumulse GML K®, and Olive oil.

Another embodiment comprises cyclosporin A, Lumulse GMO K®, and Olive oil.

Another embodiment comprises cyclosporin A, Lumulse GMR K®, and Olive oil.

Another embodiment comprises cyclosporin A, Lumulse GMT K®, and Olive oil.

Another embodiment comprises cyclosporin A, Magrabar GMC®, and Olive oil.

Another embodiment comprises cyclosporin A, Magrabar GMO-CK®, and Olive oil.

Another embodiment comprises cyclosporin A, Magrabar MDG-5050®, and Olive oil.

Another embodiment comprises cyclosporin A, Magrabar PGO-315®, and Olive oil.

Another embodiment comprises cyclosporin A, Magrabar PGO-1010®, and Olive oil.

Another embodiment comprises cyclosporin A, Mazol 300K®, and Olive oil.

Another embodiment comprises cyclosporin A, Mazol GMO-K®, and Olive oil.

Another embodiment comprises cyclosporin A, Mazol GMS-K®, and Olive oil.

Another embodiment comprises cyclosporin A, Mazol PG031-K®, and Olive oil.

Another embodiment comprises cyclosporin A, Miglyol 812®, and Olive oil.

Another embodiment comprises cyclosporin A, Norfox 165C®, and Olive oil.

Another embodiment comprises cyclosporin A, Schercemol GMIS®, and Olive oil.

Another embodiment comprises cyclosporin A, Tegin®, and Olive oil.

Another embodiment comprises cyclosporin A, Tegin 4100 Pellets®, and Olive oil.

Another embodiment comprises cyclosporin A, Tegin M Pellets®, and Olive oil.

Another embodiment comprises cyclosporin A, Tegin OV®, and Olive oil.

Another embodiment comprises cyclosporin A, Teginacid H®, and Olive oil.

Another embodiment comprises cyclosporin A, Tego Cosmo P813®, and Olive oil.

Another embodiment comprises cyclosporin A, Wickenol 535®, and Olive oil.

Another embodiment comprises cyclosporin A, Witconol 14®, and Olive oil.

Another embodiment comprises cyclosporin A, Witconol 14®, and Olive oil.

Another embodiment comprises cyclosporin A, Witconol 14F®, and Olive oil.

Another embodiment comprises cyclosporin A, Witconol 18L®, and Olive oil.

Another embodiment comprises cyclosporin A, Witconol GOT®, and Olive oil.

Another embodiment comprises cyclosporin A, Witconol MST®, and Olive oil.

Another embodiment comprises cyclosporin A, Witconol RHT®, and Olive oil.

Another embodiment comprises cyclosporin A, a Glycol Ester, and Olive oil.

Another embodiment comprises cyclosporin A, Alkamuls 600 DO®, and Olive oil.

Another embodiment comprises cyclosporin A, Alkamuls SEG®, and Olive oil.

Another embodiment comprises cyclosporin A, Atlas EM-2®, and Olive oil.

Another embodiment comprises cyclosporin A, Cerasynt IP®, and Olive oil.

Another embodiment comprises cyclosporin A, Cerasynt M®, and Olive oil.

Another embodiment comprises cyclosporin A, Cerasynt MN®, and Olive oil.

Another embodiment comprises cyclosporin A, Cerasynt PA®, and Olive oil.

Another embodiment comprises cyclosporin A, Chemsperse EGDS®, and Olive oil.

Another embodiment comprises cyclosporin A, Chemsperse EGMS®, and Olive oil.

Another embodiment comprises cyclosporin A, Colonial Monolaurin®, and Olive oil.

Another embodiment comprises cyclosporin A, DeMuls SGE-95®, and Olive oil.

Another embodiment comprises cyclosporin A, Eccoterge 200®, and Olive oil.

Another embodiment comprises cyclosporin A, Emerest 2380®, and Olive oil.

Another embodiment comprises cyclosporin A, Ethox 2610®, and Olive oil.

Another embodiment comprises cyclosporin A, Ethox DO-9®, and Olive oil.

Another embodiment comprises cyclosporin A, Ethox DO-14®, and Olive oil.

Another embodiment comprises cyclosporin A, Ethox SO-9®, and Olive oil.

Another embodiment comprises cyclosporin A, Fizul MD-318®, and Olive oil.

Another embodiment comprises cyclosporin A, Genapol EGDS-VHP®, and Olive oil.

Another embodiment comprises cyclosporin A, Genapol TS Powder®, and Olive oil.

Another embodiment comprises cyclosporin A, Hostacerin WO®, and Olive oil.

Another embodiment comprises cyclosporin A, Inversol 140®, and Olive oil.

Another embodiment comprises cyclosporin A, Kemester 104®, and Olive oil.

Another embodiment comprises cyclosporin A, Kemester 205®, and Olive oil.

Another embodiment comprises cyclosporin A, Kemester 226®, and Olive oil.

Another embodiment comprises cyclosporin A, Kemester 5221 SE®, and Olive oil.

Another embodiment comprises cyclosporin A, Kemester EGDS®, and Olive oil.

Another embodiment comprises cyclosporin A, Lexemul EGDS®, and Olive oil.

Another embodiment comprises cyclosporin A, Lexemul EGMS®, and Olive oil.

Another embodiment comprises cyclosporin A, Lexemul P®, and Olive oil.

Another embodiment comprises cyclosporin A, Lipo DGLS®, and Olive oil.

Another embodiment comprises cyclosporin A, Lipo EGDS®, and Olive oil.

Another embodiment comprises cyclosporin A, Lipo PGMS®, and Olive oil.

Another embodiment comprises cyclosporin A, Liposorb S-4®, and Olive oil.

Another embodiment comprises cyclosporin A, Liposorb TO-20®, and Olive oil.

Another embodiment comprises cyclosporin A, Lumulse PGO®, and Olive oil.

Another embodiment comprises cyclosporin A, Mackester EGDS®, and Olive oil.

Another embodiment comprises cyclosporin A, Mackester EGMS®, and Olive oil.

Another embodiment comprises cyclosporin A, Mackester GSTP®, and Olive oil.

Another embodiment comprises cyclosporin A, Mackester Series®, and Olive oil.

Another embodiment comprises cyclosporin A, Magrabar PDG-50®, and Olive oil.

Another embodiment comprises cyclosporin A, Mapeg 6000 DS®, and Olive oil.

Another embodiment comprises cyclosporin A, Marlowet 4702®, and Olive oil.

Another embodiment comprises cyclosporin A, Monalube 305®, and Olive oil.

Another embodiment comprises cyclosporin A, Monalube 310®, and Olive oil.

Another embodiment comprises cyclosporin A, Monalube 315®, and Olive oil.

Another embodiment comprises cyclosporin A, Monalube 320®, and Olive oil.

Another embodiment comprises cyclosporin A, Monalube 325®, and Olive oil.

Another embodiment comprises cyclosporin A, Monalube 330®, and Olive oil.

Another embodiment comprises cyclosporin A, Naturechem PGHS®, and Olive oil.

Another embodiment comprises cyclosporin A, Polycastorol PLO-840®, and Olive oil.

Another embodiment comprises cyclosporin A, Polytex 10M®, and Olive oil.

Another embodiment comprises cyclosporin A, Ritasynt IP®, and Olive oil.

Another embodiment comprises cyclosporin A, Ross Chem PEG 600 DT®, and Olive oil.

Another embodiment comprises cyclosporin A, Schercemol PGMS®, and Olive oil.

Another embodiment comprises cyclosporin A, Sponto H-44C®, and Olive oil.

Another embodiment comprises cyclosporin A, Tegin G®, and Olive oil.

Another embodiment comprises cyclosporin A, Witbreak DGE-182®, and Olive oil.

Another embodiment comprises cyclosporin A, Witbreak DGE-182®, and Olive oil.

Another embodiment comprises cyclosporin A, Witbreak DRA-21®, and Olive oil.

Another embodiment comprises cyclosporin A, Witbreak DRA-21®, and Olive oil.

Another embodiment comprises cyclosporin A, Witbreak DRA-50®, and Olive oil.

Another embodiment comprises cyclosporin A, Witbreak DRA-50®, and Olive oil.

Another embodiment comprises cyclosporin A, Witconol F26-46®, and Olive oil.

Another embodiment comprises cyclosporin A, Witconol H-32®, and Olive oil.

Another embodiment comprises cyclosporin A, Witconol H-33®, and Olive oil.

Another embodiment comprises cyclosporin A, Witconol H-35A®, and Olive oil.

Another embodiment comprises cyclosporin A, Witconol RHP®, and Olive oil.

Another embodiment comprises cyclosporin A, a Lanolin-based Derivative, and Olive oil.

Another embodiment comprises cyclosporin A, Amerchol CAB®, and Olive oil.

Another embodiment comprises cyclosporin A, Amerchol L-101®, and Olive oil.

Another embodiment comprises cyclosporin A, Amerlate LFA-LO®, and Olive oil.

Another embodiment comprises cyclosporin A, Amerlate P®, and Olive oil.

Another embodiment comprises cyclosporin A, Barre Common Degrase®, and Olive oil.

Another embodiment comprises cyclosporin A, Cholesterol NF®, and Olive oil.

Another embodiment comprises cyclosporin A, Crodalan AWS®, and Olive oil.

Another embodiment comprises cyclosporin A, Crodalan LA®, and Olive oil.

Another embodiment comprises cyclosporin A, Emery 1650®, and Olive oil.

Another embodiment comprises cyclosporin A, Emery 1650®, and Olive oil.

Another embodiment comprises cyclosporin A, Emery 1740®, and Olive oil.

Another embodiment comprises cyclosporin A, Emery 1740®, and Olive oil.

Another embodiment comprises cyclosporin A, Forlan 500®, and Olive oil.

Another embodiment comprises cyclosporin A, Forlan L®, and Olive oil.

Another embodiment comprises cyclosporin A, Laneto 50®, and Olive oil.

Another embodiment comprises cyclosporin A, Laneto 100®, and Olive oil.

Another embodiment comprises cyclosporin A, Laneto AWS®, and Olive oil.

Another embodiment comprises cyclosporin A, Lanfrax 1776®, and Olive oil.

Another embodiment comprises cyclosporin A, Lanfrax 1776®, and Olive oil.

Another embodiment comprises cyclosporin A, Lanogel 21®, and Olive oil.

Another embodiment comprises cyclosporin A, Lipolan®, and Olive oil.

Another embodiment comprises cyclosporin A, Lipolan 31®, and Olive oil.

Another embodiment comprises cyclosporin A, OHlan®, and Olive oil.

Another embodiment comprises cyclosporin A, Polychol 5®, and Olive oil.

Another embodiment comprises cyclosporin A, Polychol 15®, and Olive oil.

Another embodiment comprises cyclosporin A, Ritacetyl®, and Olive oil.

Another embodiment comprises cyclosporin A, Ritachol®, and Olive oil.

Another embodiment comprises cyclosporin A, Ritahydrox®, and Olive oil.

Another embodiment comprises cyclosporin A, Ritalafa®, and Olive oil.

Another embodiment comprises cyclosporin A, Ritalan®, and Olive oil.

Another embodiment comprises cyclosporin A, Ritalan AWS®, and Olive oil.

Another embodiment comprises cyclosporin A, Ritalan C®, and Olive oil.

Another embodiment comprises cyclosporin A, Ritawax®, and Olive oil.

Another embodiment comprises cyclosporin A, Ritawax AEO®, and Olive oil.

Another embodiment comprises cyclosporin A, Ritawax ALA®, and Olive oil.

Another embodiment comprises cyclosporin A, Solan/Solan 50/Super Solan®, and Olive oil.

Another embodiment comprises cyclosporin A, Super Hartolan/Hartolan®, and Olive oil.

Another embodiment comprises cyclosporin A, Supersat AWS-4®, and Olive oil.

Another embodiment comprises cyclosporin A, Supersat AWS-24®, and Olive oil.

Another embodiment comprises cyclosporin A, a Lecithin or Lecithin Derivative, and Olive oil.

Another embodiment comprises cyclosporin A, Alcolec®, and Olive oil.

Another embodiment comprises cyclosporin A, Lecithin®, and Olive oil.

Another embodiment comprises cyclosporin A, Lexin K®, and Olive oil.

Another embodiment comprises cyclosporin A, Natipide®, and Olive oil.

Another embodiment comprises cyclosporin A, a Lignin or Lignin Derivative, and Olive oil.

Another embodiment comprises cyclosporin A, Diwatex XP 9®, and Olive oil.

Another embodiment comprises cyclosporin A, Dynasperse LCD®, and Olive oil.

Another embodiment comprises cyclosporin A, Indulin SAL®, and Olive oil.

Another embodiment comprises cyclosporin A, Indulin W-1®, and Olive oil.

Another embodiment comprises cyclosporin A, Indulin W-5®, and Olive oil.

Another embodiment comprises cyclosporin A, Lignosol FTA®, and Olive oil.

Another embodiment comprises cyclosporin A, Lignosol SFX-65®, and Olive oil.

Another embodiment comprises cyclosporin A, Marasperse 52 CP®, and Olive oil.

Another embodiment comprises cyclosporin A, Marasperse AG®, and Olive oil.

Another embodiment comprises cyclosporin A, Marasperse CBOS-4®, and Olive oil.

Another embodiment comprises cyclosporin A, Ufoxane 2®, and Olive oil.

Another embodiment comprises cyclosporin A, a Methyl Ester, and Olive oil.

Another embodiment comprises cyclosporin A, E.B. Cleaner AK®, and Olive oil.

Another embodiment comprises cyclosporin A, Oleocal ME-70®, and Olive oil.

Another embodiment comprises cyclosporin A, Trade Name Company®, and Olive oil.

Another embodiment comprises cyclosporin A, Methyl Esters (cont'd)®, and Olive oil.

Another embodiment comprises cyclosporin A, Oleocal ME-92®, and Olive oil.

Another embodiment comprises cyclosporin A, Oleocal ME-112®, and Olive oil.

Another embodiment comprises cyclosporin A, Oleocal ME-130®, and Olive oil.

Another embodiment comprises cyclosporin A, a Monoglyceride or a Derivative thereof, and Olive oil.

Another embodiment comprises cyclosporin A, Dynacet 211®, and Olive oil.

Another embodiment comprises cyclosporin A, Hetsorb S-20®, and Olive oil.

Another embodiment comprises cyclosporin A, Imwitor 191®, and Olive oil.

Another embodiment comprises cyclosporin A, Imwitor 370®, and Olive oil.

Another embodiment comprises cyclosporin A, Imwitor 375®, and Olive oil.

Another embodiment comprises cyclosporin A, Imwitor 900®, and Olive oil.

Another embodiment comprises cyclosporin A, Imwitor 945®, and Olive oil.

Another embodiment comprises cyclosporin A, Imwitor 2020®, and Olive oil.

Another embodiment comprises cyclosporin A, Kemester 5500®, and Olive oil.

Another embodiment comprises cyclosporin A, Kemester 6000®, and Olive oil.

Another embodiment comprises cyclosporin A, Magrabar GMC®, and Olive oil.

Another embodiment comprises cyclosporin A, Magrabar GMO-CK®, and Olive oil.

Another embodiment comprises cyclosporin A, Magrabar GPC-1®, and Olive oil.

Another embodiment comprises cyclosporin A, Magrabar MDG-5050®, and Olive oil.

Another embodiment comprises cyclosporin A, Monalube 335®, and Olive oil.

Another embodiment comprises cyclosporin A, Monoglycerides Glyceryl Monestearate Archer Daniels Midland Company®, and Olive oil.

Another embodiment comprises cyclosporin A, Rita GMS®, and Olive oil.

Another embodiment comprises cyclosporin A, Ritamulse SCG®, and Olive oil.

Another embodiment comprises cyclosporin A, Softigen 701®, and Olive oil.

Another embodiment comprises cyclosporin A, Tally 100 Plus®, and Olive oil.

Another embodiment comprises cyclosporin A, a Polyethylene Glycol®, and Olive oil.

Another embodiment comprises cyclosporin A, Emulgade PL 68/50®, and Olive oil.

Another embodiment comprises cyclosporin A, Lumulse PEG®, and Olive oil.

Another embodiment comprises cyclosporin A, Rhodasurf PEG-400®, and Olive oil.

Another embodiment comprises cyclosporin A, Rhodasurf PEG-600®, and Olive oil.

Another embodiment comprises cyclosporin A, Witconol PEG-400®, and Olive oil.

Another embodiment comprises cyclosporin A, a Polymeric Surfactant, and Olive oil.

Another embodiment comprises cyclosporin A, Acritamer PNC-EG®, and Olive oil.

Another embodiment comprises cyclosporin A, Ag-Rho DEP-775®, and Olive oil.

Another embodiment comprises cyclosporin A, APG 325N Glycoside®, and Olive oil.

Another embodiment comprises cyclosporin A, Aristoflex AVC®, and Olive oil.

Another embodiment comprises cyclosporin A, Aristoflex HMB®, and Olive oil.

Another embodiment comprises cyclosporin A, Burco NPS-225®, and Olive oil.

Another embodiment comprises cyclosporin A, Burco NPS-816®, and Olive oil.

Another embodiment comprises cyclosporin A, Chemccinate 5603®, and Olive oil.

Another embodiment comprises cyclosporin A, Cosmedia Guar C-261 N®, and Olive oil.

Another embodiment comprises cyclosporin A, Gantrez S-95®, and Olive.oil.

Another embodiment comprises cyclosporin A, Glucopon 220 UP®, and Olive oil.

Another embodiment comprises cyclosporin A, Glucopon 225 DK®, and Olive oil.

Another embodiment comprises cyclosporin A, Glucopon 425 N®, and Olive oil.

Another embodiment comprises cyclosporin A, Glucopon 600 UP®, and Olive oil.

Another embodiment comprises cyclosporin A, Glucopon 625 UP®, and Olive oil.

Another embodiment comprises cyclosporin A, Pemulen 1621®, and Olive oil.

Another embodiment comprises cyclosporin A, Pemulen 1622®, and Olive oil.

Another embodiment comprises cyclosporin A, Pemulen TR-1®, and Olive oil.

Another embodiment comprises cyclosporin A, Pemulen TR-2®, and Olive oil.

Another embodiment comprises cyclosporin A, Plantacare 818®, and Olive oil.

Another embodiment comprises cyclosporin A, Plantapon LGC Sorb®, and Olive oil.

Another embodiment comprises cyclosporin A, Plantaren 1200N®, and Olive oil.

Another embodiment comprises cyclosporin A, Plantaren 2000N®, and Olive oil.

Another embodiment comprises cyclosporin A, Viscolam AT 64®, and Olive oil.

Another embodiment comprises cyclosporin A, Viscolam AT 64P®, and Olive oil.

Another embodiment comprises cyclosporin A, Viscolam AT 100®, and Olive oil.

Another embodiment comprises cyclosporin A, Viscolam MAC 7®, and Olive oil.

Another embodiment comprises cyclosporin A, Viscolam SMC 20®, and Olive oil.

Another embodiment comprises cyclosporin A, Witbreak RTC-323®, and Olive oil.

Another embodiment comprises cyclosporin A, WSI 370®, and Olive oil.

Another embodiment comprises cyclosporin A, a Propoxylated or Ethoxylated Fatty Acid, Alcohol, or Alkyl Phenol, and Olive oil.

Another embodiment comprises cyclosporin A, Antarox AA-60®, and Olive oil.

Another embodiment comprises cyclosporin A, Antarox LF-224®, and Olive oil.

Another embodiment comprises cyclosporin A, Burcomul DFE-45®, and Olive oil.

Another embodiment comprises cyclosporin A, Burcoterge LFE-1000®, and Olive oil.

Another embodiment comprises cyclosporin A, Chemal LF-25B®, and Olive oil.

Another embodiment comprises cyclosporin A, Chemal LF-40B®, and Olive oil.

Another embodiment comprises cyclosporin A, Dehypon LS-36®, and Olive oil.

Another embodiment comprises cyclosporin A, Dehypon LS-36®, and Olive oil.

Another embodiment comprises cyclosporin A, Dehypon LS-54®, and Olive oil.

Another embodiment comprises cyclosporin A, Dehypon LS-54®, and Olive oil.

Another embodiment comprises cyclosporin A, Delonic 100 VLF®, and Olive oil.

Another embodiment comprises cyclosporin A, Delonic LF-60 MOD®, and Olive oil.

Another embodiment comprises cyclosporin A, Empiderm B®, and Olive oil.

Another embodiment comprises cyclosporin A, Ethylan 1206®, and Olive oil.

Another embodiment comprises cyclosporin A, Ethylan NS-500K®, and Olive oil.

Another embodiment comprises cyclosporin A, Ethylan NS-500LQ®, and Olive oil.

Another embodiment comprises cyclosporin A, Genapol 1392®, and Olive oil.

Another embodiment comprises cyclosporin A, Genapol 2317®, and Olive oil.

Another embodiment comprises cyclosporin A, Genapol 26EP710®, and Olive oil.

Another embodiment comprises cyclosporin A, Genapol EP 1022®, and Olive oil.

Another embodiment comprises cyclosporin A, Genapol EP 1024®, and Olive oil.

Another embodiment comprises cyclosporin A, Genapol EP 6068®, and Olive oil.

Another embodiment comprises cyclosporin A, Genapol NP915®, and Olive oil.

Another embodiment comprises cyclosporin A, Kieralon MFB®, and Olive oil.

Another embodiment comprises cyclosporin A, Lumisolve CSA-80 V®, and Olive oil.

Another embodiment comprises cyclosporin A, Marlowet 5001®, and Olive oil.

Another embodiment comprises cyclosporin A, Marlox FK 64®, and Olive oil.

Another embodiment comprises cyclosporin A, Marlox MO 124®, and Olive oil.

Another embodiment comprises cyclosporin A, Marlox S 58®, and Olive oil.

Another embodiment comprises cyclosporin A, Nonatell 1003®, and Olive oil.

Another embodiment comprises cyclosporin A, Nonatell 1038®, and Olive oil.

Another embodiment comprises cyclosporin A, Nonatell 1052®, and Olive oil:

Another embodiment comprises cyclosporin A, Nonatell 1061®, and Olive oil.

Another embodiment comprises cyclosporin A, Nonatell 1075®, and Olive oil.

Another embodiment comprises cyclosporin A, Nonatell 1088®, and Olive oil.

Another embodiment comprises cyclosporin A, Nonatell 1123®, and Olive oil.

Another embodiment comprises cyclosporin A, Nonatell 1153®, and Olive oil.

Another embodiment comprises cyclosporin A, Nonatell 1161®, and Olive oil.

Another embodiment comprises cyclosporin A, Nonatell 1172®, and Olive oil.

Another embodiment comprises cyclosporin A, Nonatell 1181®, and Olive oil.

Another embodiment comprises cyclosporin A, Nonox 36®, and Olive oil.

Another embodiment comprises cyclosporin A, Procetyl AWS®, and Olive oil.

Another embodiment comprises cyclosporin A, Sandoxylate SX 412®, and Olive oil.

Another embodiment comprises cyclosporin A, Sandoxylate SX 418®, and Olive oil.

Another embodiment comprises cyclosporin A, Surfonic JL-80X®, and Olive oil.

Another embodiment comprises cyclosporin A, Surfonic JL-80X-B1®, and Olive oil.

Another embodiment comprises cyclosporin A, Surfonic L4-29X®, and Olive oil.

Another embodiment comprises cyclosporin A, Surfonic LF®, and Olive oil.

Another embodiment comprises cyclosporin A, T-Det A826®, and Olive oil.

Another embodiment comprises cyclosporin A, T-Det LF-416®, and Olive oil.

Another embodiment comprises cyclosporin A, Tergitol Min-Foam 1X®, and Olive oil.

Another embodiment comprises cyclosporin A, Tergitol Min-Foam 2X®, and Olive oil.

Another embodiment comprises cyclosporin A, Triton CF-21®, and Olive oil.

Another embodiment comprises cyclosporin A, Triton CF-76®, and Olive oil.

Another embodiment comprises cyclosporin A, Triton XL-80N®, and Olive oil.

Another embodiment comprises cyclosporin A, Witconol NS-98®, and Olive oil.

Another embodiment comprises cyclosporin A, Witconol NS-108LQ®, and Olive oil.

Another embodiment comprises cyclosporin A, Witconol NS-145®, and Olive oil.

Another embodiment comprises cyclosporin A, Witconol NS-179®, and Olive oil.

Another embodiment comprises cyclosporin A, a Protein-based Surfactant, and Olive oil.

Another embodiment comprises cyclosporin A, AminoFoam W®, and Olive oil.

Another embodiment comprises cyclosporin A, Amiter LGOD-2®, and Olive oil.

Another embodiment comprises cyclosporin A, Amiter LGS-2®, and Olive oil.

Another embodiment comprises cyclosporin A, Amiter LGS-5®, and Olive oil.

Another embodiment comprises cyclosporin A, Lamepon S®, and Olive oil.

Another embodiment comprises cyclosporin A, Lamepon S®, and Olive oil.

Another embodiment comprises cyclosporin A, Maypon 4C®, and Olive oil.

Another embodiment comprises cyclosporin A, May-Tein C®, and Olive oil.

Another embodiment comprises cyclosporin A, May-Tein CT®, and Olive oil.

Another embodiment comprises cyclosporin A, May-Tein KTS®, and Olive oil.

Another embodiment comprises cyclosporin A, May-Tein SY®, and Olive oil.

Another embodiment comprises cyclosporin A, Plantapon S®, and Olive oil.

Another embodiment comprises cyclosporin A, Proteol APL®, and Olive oil.

Another embodiment comprises cyclosporin A, Proteol OAT®, and Olive oil.

Another embodiment comprises cyclosporin A, Pyroter CPI-40®, and Olive oil.

Another embodiment comprises cyclosporin A, Pyroter GPI-25®, and Olive oil.

Another embodiment comprises cyclosporin A, Supro-Tein S®, and Olive oil.

Another embodiment comprises cyclosporin A, Supro-Tein V®, and Olive oil.

Another embodiment comprises cyclosporin A,® and Olive oil.

Another embodiment comprises cyclosporin A, a Sarcosine Derivative®, and Olive oil.

Another embodiment comprises cyclosporin A, Crodasinic LS-30®, and Olive oil.

Another embodiment comprises cyclosporin A, Vanseal CS®, and Olive oil.

Another embodiment comprises cyclosporin A, Vanseal LS®, and Olive oil.

Another embodiment comprises cyclosporin A, Vanseal MS®, and Olive oil.

Another embodiment comprises cyclosporin A, Vanseal NACS-30®, and Olive oil.

Another embodiment comprises cyclosporin A, Vanseal NALS-95®, and Olive oil.

Another embodiment comprises cyclosporin A, Vanseal OS®, and Olive oil.

Another embodiment comprises cyclosporin A,® and Olive oil.

Another embodiment comprises cyclosporin A, a Silicone-based Surfactant®, and Olive oil.

Another embodiment comprises cyclosporin A, Abil-B-9950®, and Olive oil.

Another embodiment comprises cyclosporin A, Abil Care 85®, and Olive oil.

Another embodiment comprises cyclosporin A, Abil EM 90®, and Olive oil.

Another embodiment comprises cyclosporin A, Abil EM 97®, and Olive oil.

Another embodiment comprises cyclosporin A, Abil WE-09®, and Olive oil.

Another embodiment comprises cyclosporin A, Dow Corning 1248 Fluid®, and Olive oil.

Another embodiment comprises cyclosporin A, Dow Corning 3225C®, and Olive oil.

Another embodiment comprises cyclosporin A, Dow Corning 5200®, and Olive oil.

Another embodiment comprises cyclosporin A, Dow Corning Q4-3667®, and Olive oil.

Another embodiment comprises cyclosporin A, Monasil PCA®, and Olive oil.

Another embodiment comprises cyclosporin A, Monasil PDM®, and Olive oil.

Another embodiment comprises cyclosporin A, Monasil PLN®, and Olive oil.

Another embodiment comprises cyclosporin A, Polyderm PPI-SI-WS®, and Olive oil.

Another embodiment comprises cyclosporin A, Troysol 380W®, and Olive oil.

Another embodiment comprises cyclosporin A, Troysol S366®, and Olive oil.

Another embodiment comprises cyclosporin A, a Sorbitan Derivative, and Olive oil.

Another embodiment comprises cyclosporin A, Alkamuls SML®, and Olive oil.

Another embodiment comprises cyclosporin A, Alkamuls SMO®, and Olive oil.

Another embodiment comprises cyclosporin A, Alkamuls STO®, and Olive oil.

Another embodiment comprises cyclosporin A, Arlacel 20®, and Olive oil.

Another embodiment comprises cyclosporin A, Arlacel 40®, and Olive oil.

Another embodiment comprises cyclosporin A, Arlacel 60®, and Olive oil.

Another embodiment comprises cyclosporin A, Arlacel 80®, and Olive oil.

Another embodiment comprises cyclosporin A, Arlacel C®, and Olive oil.

Another embodiment comprises cyclosporin A, Armul 21®, and Olive oil.

Another embodiment comprises cyclosporin A, Atlox 80®, and Olive oil.

Another embodiment comprises cyclosporin A, Atlox 847®, and Olive oil.

Another embodiment comprises cyclosporin A, Atlox 1045A®, and Olive oil.

Another embodiment comprises cyclosporin A, Canarcel 20®, and Olive oil.

Another embodiment comprises cyclosporin A, Canarcel 60®, and Olive oil.

Another embodiment comprises cyclosporin A, Canarcel 80®, and Olive oil.

Another embodiment comprises cyclosporin A, Canarcel TW 20®, and Olive oil.

Another embodiment comprises cyclosporin A, Canarcel TW 60®, and Olive oil.

Another embodiment comprises cyclosporin A, Canarcel TW 80®, and Olive oil.

Another embodiment comprises cyclosporin A, Coladet BSB-P®, and Olive oil.

Another embodiment comprises cyclosporin A, Customulse O-20®, and Olive oil.

Another embodiment comprises cyclosporin A, Dehymuls E®, and Olive oil.

Another embodiment comprises cyclosporin A, DeSotan SMO®, and Olive oil.

Another embodiment comprises cyclosporin A, DeSotan SMO-20®, and Olive oil.

Another embodiment comprises cyclosporin A, DeSotan SMT®, and Olive oil.

Another embodiment comprises cyclosporin A, DeSotan SMT-20®, and Olive oil.

Another embodiment comprises cyclosporin A, Durfax 60®, and Olive oil.

Another embodiment comprises cyclosporin A, Durfax 65®, and Olive oil.

Another embodiment comprises cyclosporin A, Durfax 80®, and Olive oil.

Another embodiment comprises cyclosporin A, Durtan 60®, and Olive oil.

Another embodiment comprises cyclosporin A, Durtan 65®, and Olive oil.

Another embodiment comprises cyclosporin A, Liposorb L®, and Olive oil.

Another embodiment comprises cyclosporin A, Liposorb L-10®, and Olive oil.

Another embodiment comprises cyclosporin A, Liposorb L-20®, and Olive oil.

Another embodiment comprises cyclosporin A, Liposorb O®, and Olive oil.

Another embodiment comprises cyclosporin A, Liposorb O-20®, and Olive oil.

Another embodiment comprises cyclosporin A, Liposorb P®, and Olive oil.

Another embodiment comprises cyclosporin A, Liposorb P-20®, and Olive oil.

Another embodiment comprises cyclosporin A, Liposorb S®, and Olive oil.

Another embodiment comprises cyclosporin A, Liposorb S-20®, and Olive oil.

Another embodiment comprises cyclosporin A, Liposorb SQO®, and Olive oil.

Another embodiment comprises cyclosporin A, Liposorb TO®, and Olive oil.

Another embodiment comprises cyclosporin A, Liposorb TS®, and Olive oil.

Another embodiment comprises cyclosporin A, Liposorb TS-20®, and Olive oil.

Another embodiment comprises cyclosporin A, Lumisorb PS®, and Olive oil.

Another embodiment comprises cyclosporin A, Lumisorb SMO (T)®, and Olive oil.

Another embodiment comprises cyclosporin A, Lumisorb SMS K®, and Olive oil.

Another embodiment comprises cyclosporin A, Lumisorb SSO®, and Olive oil.

Another embodiment comprises cyclosporin A, Lumisorb STS K®, and Olive oil.

Another embodiment comprises cyclosporin A, Lumisorb STT®, and Olive oil.

Another embodiment comprises cyclosporin A, Magrabar SMO®, and Olive oil.

Another embodiment comprises cyclosporin A, Magrabar SMO-VEG®, and Olive oil.

Another embodiment comprises cyclosporin A, Magrabar SMT®, and Olive oil.

Another embodiment comprises cyclosporin A, Magrabar STO®, and Olive oil.

Another embodiment comprises cyclosporin A, Miracare BC-27®, and Olive oil.

Another embodiment comprises cyclosporin A, Ritabate 20®, and Olive oil.

Another embodiment comprises cyclosporin A, Ritabate 40®, and Olive oil.

Another embodiment comprises cyclosporin A, Ritabate 60®, and Olive oil.

Another embodiment comprises cyclosporin A, Ritabate 80®, and Olive oil.

Another embodiment comprises cyclosporin A, T-Maz®, and Olive oil.

Another embodiment comprises cyclosporin A, Tego SML®, and Olive oil.

Another embodiment comprises cyclosporin A, Tego SML 20®, and Olive oil.

Another embodiment comprises cyclosporin A, Tego SMO 80 V®, and Olive oil.

Another embodiment comprises cyclosporin A, Tego SMO V®, and Olive oil.

Another embodiment comprises cyclosporin A, Tego SMS®, and Olive oil.

Another embodiment comprises cyclosporin A, Tego STO V®, and Olive oil.

Another embodiment comprises cyclosporin A, Tween 21®, and Olive oil.

Another embodiment comprises cyclosporin A, Tween 40®, and Olive oil.

Another embodiment comprises cyclosporin A, Tween 60®, and Olive oil.

Another embodiment comprises cyclosporin A, Tween 60 K®, and Olive oil.

Another embodiment comprises cyclosporin A, Tween 61®, and Olive oil.

Another embodiment comprises cyclosporin A, Tween 65®, and Olive oil.

Another embodiment comprises cyclosporin A, Tween 80®, and Olive oil.

Another embodiment comprises cyclosporin A, Tween 80 K®, and Olive oil.

Another embodiment comprises cyclosporin A, Tween 81®, and Olive oil.

Another embodiment comprises cyclosporin A, Tween 85®, and Olive oil.

Another embodiment comprises cyclosporin A, a Sucrose or Glucose Ester, or Derivative thereof, and Olive oil.

Another embodiment comprises cyclosporin A, DeSulf GOS-P-60WCG®, and Olive oil.

Another embodiment comprises cyclosporin A, Glucam E-20 Distearate®, and Olive oil.

Another embodiment comprises cyclosporin A, Glucamate DOE-120®, and Olive oil.

Another embodiment comprises cyclosporin A, Glucamate SSE-20®, and Olive oil.

Another embodiment comprises cyclosporin A, Glucate DO®, and Olive oil.

Another embodiment comprises cyclosporin A, Glucate SS®, and Olive oil.

Another embodiment comprises cyclosporin A, Glucopon 425 UP®, and Olive oil.

Another embodiment comprises cyclosporin A, Isolan IS®, and Olive oil.

Another embodiment comprises cyclosporin A, Mazon 40®, and Olive oil.

Another embodiment comprises cyclosporin A, Montanov 82®, and Olive oil.

Another embodiment comprises cyclosporin A, Montanov 202®, and Olive oil.

Another embodiment comprises cyclosporin A, Montanov S®, and Olive oil.

Another embodiment comprises cyclosporin A, Rheozan®, and Olive oil.

Another embodiment comprises cyclosporin A, Simulsol AS 48®, and Olive oil.

Another embodiment comprises cyclosporin A, Simulsol SL 4®, and Olive oil.

Another embodiment comprises cyclosporin A, Simulsol SL 10®, and Olive oil.

Another embodiment comprises cyclosporin A, Simulsol SL 11W®, and Olive oil.

Another embodiment comprises cyclosporin A, Simulsol SL 55®, and Olive oil.

Another embodiment comprises cyclosporin A, Suga Nate 100 and 160®, and Olive oil.

Another embodiment comprises cyclosporin A, Tego Care 450®, and Olive oil.

Another embodiment comprises cyclosporin A, Tego Care CG 90®, and Olive oil.

Another embodiment comprises cyclosporin A, Tego Care PS®, and Olive oil.

Another embodiment comprises cyclosporin A, Tegosoft PSE 141 G®, and Olive oil.

Another embodiment comprises cyclosporin A, Tegotens G 826®, and Olive oil.

Another embodiment comprises cyclosporin A, Triton BG-10 (70%)®, and Olive oil.

Another embodiment comprises cyclosporin A, Triton CG-110 (60%)®, and Olive oil.

Another embodiment comprises cyclosporin A, Wickenol 545®, and Olive oil.

Another embodiment comprises cyclosporin A, an alcohol, and Caraway oil.

Another embodiment comprises cyclosporin A, Diglycerol®, and Caraway oil.

Another embodiment comprises cyclosporin A, Hetoxide GT-80®, and Caraway oil.

Another embodiment comprises cyclosporin A, Lexemul BEO®, and Caraway oil.

Another embodiment comprises cyclosporin A, Polyglycerol-3®, and Caraway oil.

Another embodiment comprises cyclosporin A, Redicote E Series®, and Caraway oil.

Another embodiment comprises cyclosporin A, Simulsol OX 1005L®, and Caraway oil.

Another embodiment comprises cyclosporin A, Stanfax 567®, and Caraway oil.

Another embodiment comprises cyclosporin A, TA-1618®, and Caraway oil.

Another embodiment comprises cyclosporin A, Witconol H-31A®, and Caraway oil.

Another embodiment comprises cyclosporin A, an amine oxide, and Caraway oil.

Another embodiment comprises cyclosporin A, AO-405®, and Caraway oil.

Another embodiment comprises cyclosporin A, AO-455®, and Caraway oil.

Another embodiment comprises cyclosporin A, AO 728 Special®, and Caraway oil.

Another embodiment comprises cyclosporin A, Barlox 12®, and Caraway oil.

Another embodiment comprises cyclosporin A, Barlox 14®, and Caraway oil.

Another embodiment comprises cyclosporin A, Burcoxide Lo®, and Caraway oil.

Another embodiment comprises cyclosporin A, Caloxamine LO®, and Caraway oil.

Another embodiment comprises cyclosporin A, Chemoxide CAW®, and Caraway oil.

Another embodiment comprises cyclosporin A, Chemoxide LM-30®, and Caraway oil.

Another embodiment comprises cyclosporin A, Chemoxide LO®, and Caraway oil.

Another embodiment comprises cyclosporin A, Chemoxide MO®, and Caraway oil.

Another embodiment comprises cyclosporin A, Colalux CAO-35®, and Caraway oil.

Another embodiment comprises cyclosporin A, Colalux LO®, and Caraway oil.

Another embodiment comprises cyclosporin A, DeMox CAPO®, and Caraway oil.

Another embodiment comprises cyclosporin A, DeMox CSG-30®, and Caraway oil.

Another embodiment comprises cyclosporin A, DeMox LAO®, and Caraway oil.

Another embodiment comprises cyclosporin A, Emcol L®, and Caraway oil.

Another embodiment comprises cyclosporin A, Empigen OB®, and Caraway oil.

Another embodiment comprises cyclosporin A, Empigen OS/A®, and Caraway oil.

Another embodiment comprises cyclosporin A, Foamox CDO®, and Caraway oil.

Another embodiment comprises cyclosporin A, Foamox DMM®, and Caraway oil.

Another embodiment comprises cyclosporin A, Foamox DMS®, and Caraway oil.

Another embodiment comprises cyclosporin A, Genaminox KC®, and Caraway oil.

Another embodiment comprises cyclosporin A, Genaminox LA®, and Caraway oil.

Another embodiment comprises cyclosporin A, Hartofoam SAO®, and Caraway oil.

Another embodiment comprises cyclosporin A, Hartox DMCD®, and Caraway oil.

Another embodiment comprises cyclosporin A, Lipowax DAT®, and Caraway oil.

Another embodiment comprises cyclosporin A, Lipowax PB Pastilles®, and Caraway oil.

Another embodiment comprises cyclosporin A, Mackamine C8®, and Caraway oil.

Another embodiment comprises cyclosporin A, Mackamine C10®, and Caraway oil.

Another embodiment comprises cyclosporin A, Mackamine C14®, and Caraway oil.

Another embodiment comprises cyclosporin A, Mackamine CAO®, and Caraway oil.

Another embodiment comprises cyclosporin A, Mackamine CO®, and Caraway oil.

Another embodiment comprises cyclosporin A, Mackamine LO®, and Caraway oil.

Another embodiment comprises cyclosporin A, Mackamine O2®, and Caraway oil.

Another embodiment comprises cyclosporin A, Mackamine SAO®, and Caraway oil.

Another embodiment comprises cyclosporin A, Mackamine SO®, and Caraway oil.

Another embodiment comprises cyclosporin A, Mazox KCAO®, and Caraway oil.

Another embodiment comprises cyclosporin A, Monalac MO®, and Caraway oil.

Another embodiment comprises cyclosporin A, Norfox LDA®, and Caraway oil.

Another embodiment comprises cyclosporin A, Rhodamox LO®, and Caraway oil.

Another embodiment comprises cyclosporin A, Schercamox C-AA®, and Caraway oil.

Another embodiment comprises cyclosporin A, Schercamox DMA®, and Caraway oil.

Another embodiment comprises cyclosporin A, Schercamox DML®, and Caraway oil.

Another embodiment comprises cyclosporin A, Schercamox DMM®, and Caraway oil.

Another embodiment comprises cyclosporin A, Schercamox DMS®, and Caraway oil.

Another embodiment comprises cyclosporin A, Tegotens DO®, and Caraway oil.

Another embodiment comprises cyclosporin A, Tomah AO-14-2®, and Caraway oil.

Another embodiment comprises cyclosporin A, Triaminox CDO®, and Caraway oil.

Another embodiment comprises cyclosporin A, a block polymer, and Caraway oil.

Another embodiment comprises cyclosporin A, AL 2070®, and Caraway oil.

Another embodiment comprises cyclosporin A, Antarox 17-R-2®, and Caraway oil.

Another embodiment comprises cyclosporin A, Antarox 25-R-2®, and Caraway oil.

Another embodiment comprises cyclosporin A, Antarox 31-R-1®, and Caraway oil.

Another embodiment comprises cyclosporin A, Antarox P-84®, and Caraway oil.

Another embodiment comprises cyclosporin A, Antarox P-104/H®, and Caraway oil.

Another embodiment comprises cyclosporin A, Arnox BP-Series®, and Caraway oil.

Another embodiment comprises cyclosporin A, Chemonic 435®, and Caraway oil.

Another embodiment comprises cyclosporin A, Chemonic D-25®, and Caraway oil.

Another embodiment comprises cyclosporin A, Chemonic PL Series®, and Caraway oil.

Another embodiment comprises cyclosporin A, Ethox L-121®, and Caraway oil.

Another embodiment comprises cyclosporin A, Ethox L-122®, and Caraway oil.

Another embodiment comprises cyclosporin A, Genapol PF-10®, and Caraway oil.

Another embodiment comprises cyclosporin A, Genapol PF-20®, and Caraway oil.

Another embodiment comprises cyclosporin A, Genapol PF-40A®, and Caraway oil.

Another embodiment comprises cyclosporin A, Norfox 2-LF®, and Caraway oil.

Another embodiment comprises cyclosporin A, Simulsol NW 342®, and Caraway oil.

Another embodiment comprises cyclosporin A, T-Det BP-1®, and Caraway oil.

Another embodiment comprises cyclosporin A, T-Det XD®, and Caraway oil.

Another embodiment comprises cyclosporin A, T-Det XH®, and Caraway oil.

Another embodiment comprises cyclosporin A, Triton CF-32®, and Caraway oil.

Another embodiment comprises cyclosporin A, Witconol 171®, and Caraway oil.

Another embodiment comprises cyclosporin A, Witconol 324®, and Caraway oil.

Another embodiment comprises cyclosporin A, Witconol 324D®, and Caraway oil.

Another embodiment comprises cyclosporin A, Witconol PD-2000®, and Caraway oil. and Caraway oil.

a Carboxylated Alcohol or Alkylphenol Ethoxylate, and Caraway oil.

Another embodiment comprises cyclosporin A, Emcol CN-6®, and Caraway oil.

Another embodiment comprises cyclosporin A, Ethcarb®, and Caraway oil.

Another embodiment comprises cyclosporin A, Gemtex WNT-Conc®, and Caraway oil.

Another embodiment comprises cyclosporih A, Incrodet TD7-C®, and Caraway oil.

Another embodiment comprises cyclosporin A, Marlinat CM 105/80®, and Caraway oil.

Another embodiment comprises cyclosporin A, Marlowet 1072®, and Caraway oil.

Another embodiment comprises cyclosporin A, Marlowet 4530®, and Caraway oil.

Another embodiment comprises cyclosporin A, Marlowet 4530 LF®, and Caraway oil.

Another embodiment comprises cyclosporin A, Marlowet 4534®, and Caraway oil.

Another embodiment comprises cyclosporin A, Marlowet 4538®, and Caraway oil.

Another embodiment comprises cyclosporin A, Marlowet 4539®, and Caraway oil.

Another embodiment comprises cyclosporin A, Marlowet 4539 LF®, and Caraway oil.

Another embodiment comprises cyclosporin A, Marlowet 4541®, and Caraway oil.

Another embodiment comprises cyclosporin A, Miranate LEC-80®, and Caraway oil.

Another embodiment comprises cyclosporin A, Sandopan B®, and Caraway oil.

Another embodiment comprises cyclosporin A, Sandopan B Modified®, and-Caraway oil.

Another embodiment comprises cyclosporin A, Sandopan LS-24 Gel®, and Caraway oil.

Another embodiment comprises cyclosporin A, Sunfine T-A®, and Caraway oil.

Another embodiment comprises cyclosporin A, a Carboxylic Acid/Fatty Acid, and Caraway oil.

Another embodiment comprises cyclosporin A, Colaterge RAM®, and Caraway oil.

Another embodiment comprises cyclosporin A, Colatrope INC®, and Caraway oil.

Another embodiment comprises cyclosporin A, Crodacid B®, and Caraway oil.

Another embodiment comprises cyclosporin A, DeTrope CA-100®, and Caraway oil.

Another embodiment comprises cyclosporin A, Latol MTO®, and Caraway oil.

Another embodiment comprises cyclosporin A, Lumulse CC-33 K®, and Caraway oil.

Another embodiment comprises cyclosporin A, Mulls 2218®, and Caraway oil.

Another embodiment comprises cyclosporin A, OL-600®, and Caraway oil.

Another embodiment comprises cyclosporin A, OL-800®, and Caraway oil.

Another embodiment comprises cyclosporin A, R-910®, and Caraway oil.

Another embodiment comprises cyclosporin A, S-210®, and Caraway oil.

Another embodiment comprises cyclosporin A, Sandopan DTC Acid®, and Caraway oil.

Another embodiment comprises cyclosporin A, Sandopan LS 24 N®, and Caraway oil.

Another embodiment comprises cyclosporin A, Sandopan MA-18®, and Caraway oil.

Another embodiment comprises cyclosporin A, an Ethoxylated Alcohol, and Caraway oil.

Another embodiment comprises cyclosporin A, Adsee 799®, and Caraway oil.

Another embodiment comprises cyclosporin A, Adsee 799®, and Caraway oil.

Another embodiment comprises cyclosporin A, Alfonic 610-3.5®, and Caraway oil.

Another embodiment comprises cyclosporin A, Alfonic 810-2®, and Caraway oil.

Another embodiment comprises cyclosporin A, Alfonic 810-6®, and Caraway oil.

Another embodiment comprises cyclosporin A, Alfonic 1012-3®, and Caraway oil.

Another embodiment comprises cyclosporin A, Alfonic 1012-5®, and Caraway oil.

Another embodiment comprises cyclosporin A, Alfonic 1216CO-1.5®, and Caraway oil.

Another embodiment comprises cyclosporin A, Alfonic 1216CO-7®, and Caraway oil.

Another embodiment comprises cyclosporin A, Alfonic 1412-3®, and Caraway oil.

Another embodiment comprises cyclosporin A, Alionic 1412-7®, and Caraway oil.

Another embodiment comprises cyclosporin A, Arlasolve 200®, and Caraway oil.

Another embodiment comprises cyclosporin A, Arlasolve 200 Liquid®, and Caraway oil.

Another embodiment comprises cyclosporin A, Armix 180-C®, and Caraway oil.

Another embodiment comprises cyclosporin A, Armix 183®, and Caraway oil.

Another embodiment comprises cyclosporin A, Armul 2404®, and Caraway oil.

Another embodiment comprises cyclosporin A, Armul 2404®, and Caraway oil.

Another embodiment comprises cyclosporin A, Atlas EMJ-C®, and Caraway oil.

Another embodiment comprises cyclosporin A, Atlas G-2109®, and Caraway oil.

Another embodiment comprises cyclosporin A, Atlas G-3886®, and Caraway oil.

Another embodiment comprises cyclosporin A, Atlas G-3890®, and Caraway oil.

Another embodiment comprises cyclosporin A, Bio Soft E-200®, and Caraway oil.

Another embodiment comprises cyclosporin A, Bio Soft E-300®, and Caraway oil.

Another embodiment comprises cyclosporin A, Bio Soft E-400®, and Caraway oil.

Another embodiment comprises cyclosporin A, Bio Soft EN 600®, and Caraway oil.

Another embodiment comprises cyclosporin A, Bio Soft TD-400®, and Caraway oil.

Another embodiment comprises cyclosporin A, Bio Soft TD-630®, and Caraway oil.

Another embodiment comprises cyclosporin A, Brij 30®, and Caraway oil.

Another embodiment comprises cyclosporin A, Brij 52®, and Caraway oil.

Another embodiment comprises cyclosporin A, Brij 56®, and Caraway oil.

Another embodiment comprises cyclosporin A, Brij 58®, and Caraway oil.

Another embodiment comprises cyclosporin A, Brij 72®, and Caraway oil.

Another embodiment comprises cyclosporin A, Brij 76®, and Caraway oil.

Another embodiment comprises cyclosporin A, Brij 78®, and Caraway oil.

Another embodiment comprises cyclosporin A, Brij 93®, and Caraway oil.

Another embodiment comprises cyclosporin A, Brij 97®, and Caraway oil.

Another embodiment comprises cyclosporin A, Brij 98®, and Caraway oil.

Another embodiment comprises cyclosporin A, Brij 700®, and Caraway oil.

Another embodiment comprises cyclosporin A, Brij 700 S®, and Caraway oil.

Another embodiment comprises cyclosporin A, Brij 721®, and Caraway oil.

Another embodiment comprises cyclosporin A, Brij 721 S®, and Caraway oil.

Another embodiment comprises cyclosporin A, Burcoterge CDG®, and Caraway oil.

Another embodiment comprises cyclosporin A, Canasol AT 600®, and Caraway oil.

Another embodiment comprises cyclosporin A, Canasol AT 800®, and Caraway oil.

Another embodiment comprises cyclosporin A, Canasol AT 1200®, and Caraway oil.

Another embodiment comprises cyclosporin A, Canasol BJ 35®, and Caraway oil.

Another embodiment comprises cyclosporin A, Canasol BJ 36®, and Caraway oil.

Another embodiment comprises cyclosporin A, Canasol BJ 52®, and Caraway oil.

Another embodiment comprises cyclosporin A, Canasol BJ 58®, and Caraway oil.

Another embodiment comprises cyclosporin A, Canasol BJ 72®, and Caraway oil.

Another embodiment comprises cyclosporin A, Canasol BJ 78®, and Caraway oil.

Another embodiment comprises cyclosporin A, Canasol BJ 98®, and Caraway oil.

Another embodiment comprises cyclosporin A, Canasol BJ 307®, and Caraway oil.

Another embodiment comprises cyclosporin A, Cerfak 1400®, and Caraway oil.

Another embodiment comprises cyclosporin A, Cetomacrogol 1000 BP®, and Caraway oil.

Another embodiment comprises cyclosporin A, Chemonic C-2®, and Caraway oil.

Another embodiment comprises cyclosporin A, Chemonic C-10®, and Caraway oil.

Another embodiment comprises cyclosporin A, Chemonic C-20®, and Caraway oil.

Another embodiment comprises cyclosporin A, Chemonic CT-12®, and Caraway oil.

Another embodiment comprises cyclosporin A, Chemonic CT-20®, and Caraway oil.

Another embodiment comprises cyclosporin A, Chemonic CT-30®, and Caraway oil.

Another embodiment comprises cyclosporin A, Chemonic CT-55®, and Caraway oil.

Another embodiment comprises cyclosporin A, Chemonic G-7®, and Caraway oil.

Another embodiment comprises cyclosporin A, Chemonic G-26®, and Caraway oil.

Another embodiment comprises cyclosporin A, Chemonic L-4®, and Caraway oil.

Another embodiment comprises cyclosporin A, Chemonic L-7®, and Caraway oil.

Another embodiment comprises cyclosporin A, Chemonic L-12®, and Caraway oil.

Another embodiment comprises cyclosporin A, Chemonic L-23®, and Caraway oil.

Another embodiment comprises cyclosporin A, Chemonic O-2®, and Caraway oil.

Another embodiment comprises cyclosporin A, Chemonic O-5®, and Caraway oil.

Another embodiment comprises cyclosporin A, Chemonic O-10®, and Caraway oil.

Another embodiment comprises cyclosporin A, Chemonic O-20®, and Caraway oil.

Another embodiment comprises cyclosporin A, Chemonic S-2®, and Caraway oil.

Another embodiment comprises cyclosporin A, Chemonic S-10®, and Caraway oil.

Another embodiment comprises cyclosporin A, Chemonic S-20®, and Caraway oil.

Another embodiment comprises cyclosporin A, Colamulse FE®, and Caraway oil.

Another embodiment comprises cyclosporin A, Cremophor A 20®, and Caraway oil.

Another embodiment comprises cyclosporin A, Cremophor SA 2®, and Caraway oil.

Another embodiment comprises cyclosporin A, Dehydol 100®, and Caraway oil.

Another embodiment comprises cyclosporin A, Dehydol O-4®, and Caraway oil.

Another embodiment comprises cyclosporin A, Delonic C-18®, and Caraway oil.

Another embodiment comprises cyclosporin A, DeSonic 6T®, and Caraway oil.

Another embodiment comprises cyclosporin A, DeSonic 9D®, and Caraway oil.

Another embodiment comprises cyclosporin A, DeSonic 9T®, and Caraway oil.

Another embodiment comprises cyclosporin A, DeSonic 12D®, and Caraway oil.

Another embodiment comprises cyclosporin A, DeSonic 12T®, and Caraway oil.

Another embodiment comprises cyclosporin A, DeSonic 15T®, and Caraway oil.

Another embodiment comprises cyclosporin A, DeSonic TDA-9®, and Caraway oil.

Another embodiment comprises cyclosporin A, DeThox GLG-7®, and Caraway oil.

Another embodiment comprises cyclosporin A, DeThox GLG-26®, and Caraway oil.

Another embodiment comprises cyclosporin A, DeThox LA-4®, and Caraway oil.

Another embodiment comprises cyclosporin A, DeThox LA-23®, and Caraway oil.

Another embodiment comprises cyclosporin A, DeThox SA-80®, and Caraway oil.

Another embodiment comprises cyclosporin A, Disponil O5®, and Caraway oil.

Another embodiment comprises cyclosporin A, Eccoterge EO-41B®, and Caraway oil.

Another embodiment comprises cyclosporin A, Empilan KA2.5/90®, and Caraway oil.

Another embodiment comprises cyclosporin A, Empilan KA5/90®, and Caraway oil.

Another embodiment comprises cyclosporin A, Empilan KM-20®, and Caraway oil.

Another embodiment comprises cyclosporin A, Empilan KM-50®, and Caraway oil.

Another embodiment comprises cyclosporin A, Empilan L-23®, and Caraway oil.

Another embodiment comprises cyclosporin A, Ethylan 25-3®, and Caraway oil.

Another embodiment comprises cyclosporin A, Ethylan 1204®, and Caraway oil.

Another embodiment comprises cyclosporin A, Ethylan DA-4®, and Caraway oil.

Another embodiment comprises cyclosporin A, Ethylan LA-230®, and Caraway oil.

Another embodiment comprises cyclosporin A, Ethylan SN®, and Caraway oil.

Another embodiment comprises cyclosporin A, Ethylan TD-60®, and Caraway oil.

Another embodiment comprises cyclosporin A, Ethylan TD-100®, and Caraway oil.

Another embodiment comprises cyclosporin A, Ethylan TD-1407®, and Caraway oil.

Another embodiment comprises cyclosporin A, Eumulgin B1®, and Caraway oil.

Another embodiment comprises cyclosporin A, Eumulgin B2®, and Caraway oil.

Another embodiment comprises cyclosporin A, Eumulgin B3®, and Caraway oil.

Another embodiment comprises cyclosporin A, Eumulgin O-10®, and Caraway oil.

Another embodiment comprises cyclosporin A, Flo Mo 80/20®, and Caraway oil.

Another embodiment comprises cyclosporin A, Flo Mo Low Foam®, and Caraway oil.

Another embodiment comprises cyclosporin A, Forlan C-24®, and Caraway oil.

Another embodiment comprises cyclosporin A, Genapol 1454®, and Caraway oil.

Another embodiment comprises cyclosporin A, Genapol BA-020®, and Caraway oil.

Another embodiment comprises cyclosporin A, Genapol BA-040®, and Caraway oil.

Another embodiment comprises cyclosporin A, Genapol C-100®, and Caraway oil.

Another embodiment comprises cyclosporin A, Genapol DA 060®, and Caraway oil.

Another embodiment comprises cyclosporin A, Genapol HS 020®, and Caraway oil.

Another embodiment comprises cyclosporin A, Genapol HS 200®, and Caraway oil.

Another embodiment comprises cyclosporin A, Genapol ID-040®, and Caraway oil.

Another embodiment comprises cyclosporin A, Genapol ID-060®, and Caraway oil.

Another embodiment comprises cyclosporin A, Genapol ID-090®, and Caraway oil.

Another embodiment comprises cyclosporin A, Genapol LA 010®, and Caraway oil.

Another embodiment comprises cyclosporin A, Genapol LA 020®, and Caraway oil.

Another embodiment comprises cyclosporin A, Genapol LA 030®, and Caraway oil.

Another embodiment comprises cyclosporin A, Genapol LA 040®, and Caraway oil.

Another embodiment comprises cyclosporin A, Genapol LA 050®, and Caraway oil.

Another embodiment comprises cyclosporin A, Genapol LA 060®, and Caraway oil.

Another embodiment comprises cyclosporin A, Genapol LA 070®, and Caraway oil.

Another embodiment comprises cyclosporin A, Genapol LA 070S®, and Caraway oil.

Another embodiment comprises cyclosporin A, Genapol LA 230®, and Caraway oil.

Another embodiment comprises cyclosporin A, Genapol O 020®, and Caraway oil.

Another embodiment comprises cyclosporin A, Genapol O 050®, and Caraway oil.

Another embodiment comprises cyclosporin A, Genapol O 100®, and Caraway oil.

Another embodiment comprises cyclosporin A, Genapol O 200®, and Caraway oil.

Another embodiment comprises cyclosporin A, Genapol SA 030®, and Caraway oil.

Another embodiment comprises cyclosporin A, Genapbl SA 120®, and Caraway oil.

Another embodiment comprises cyclosporin A, Genapol T-020®, and Caraway oil.

Another embodiment comprises cyclosporin A, Genapol UD-030®, and Caraway oil.

Another embodiment comprises cyclosporin A, Genapol UD-050®, and Caraway oil.

Another embodiment comprises cyclosporin A, Genapol UD-070®, and Caraway oil.

Another embodiment comprises cyclosporin A, Genapol UD-079®, and Caraway oil.

Another embodiment comprises cyclosporin A, Genapol UD-080®, and Caraway oil.

Another embodiment comprises cyclosporin A, Genapol UD-110®, and Caraway oil.

Another embodiment comprises cyclosporin A, Genapol X 030®, and Caraway oil.

Another embodiment comprises cyclosporin A, Genapol X 050®, and Caraway oil.

Another embodiment comprises cyclosporin A, Genapol X 060®, and Caraway oil.

Another embodiment comprises cyclosporin A, Genapol X 070®, and Caraway oil.

Another embodiment comprises cyclosporin A, Genapol X 080®, and Caraway oil.

Another embodiment comprises cyclosporin A, Genapol X 100®, and Caraway oil.

Another embodiment comprises cyclosporin A, Genapol X159®, and Caraway oil.

Another embodiment comprises cyclosporin A, Generol 122 E5®, and Caraway oil.

Another embodiment comprises cyclosporin A, Generol 122 E25®, and Caraway oil.

Another embodiment comprises cyclosporin A, Hostacerin T-3®, and Caraway oil.

Another embodiment comprises cyclosporin A, Iconol LF 110®, and Caraway oil.

Another embodiment comprises cyclosporin A, Incropol CS-20®, and Caraway oil.

Another embodiment comprises cyclosporin A, Lexemul CS-20®, and Caraway oil.

Another embodiment comprises cyclosporin A, Liponic EG-1®, and Caraway oil.

Another embodiment comprises cyclosporin A, Lipowax D®, and Caraway oil.

Another embodiment comprises cyclosporin A, Lipowax G®, and Caraway oil.

Another embodiment comprises cyclosporin A, Lipowax NI®, and Caraway oil.

Another embodiment comprises cyclosporin A, Lipowax P®, and Caraway oil.

Another embodiment comprises cyclosporin A, Lipowax P-31®, and Caraway oil.

Another embodiment comprises cyclosporin A, Lipowax PR®, and Caraway oil.

Another embodiment comprises cyclosporin A, Lumulse CS-20®, and Caraway oil.

Another embodiment comprises cyclosporin A, Macol CSA-20®, and Caraway oil.

Another embodiment comprises cyclosporin A, Marlox B 24/50®, and Caraway oil.

Another embodiment comprises cyclosporin A, Mazawet 77®, and Caraway oil.

Another embodiment comprises cyclosporin A, Norfox 1713®, and Caraway oil.

Another embodiment comprises cyclosporin A, Norfox 2579®, and Caraway oil.

Another embodiment comprises cyclosporin A, Norfox Lo Foam®, and Caraway oil.

Another embodiment comprises cyclosporin A, Prbmulgen D®, and Caraway oil.

Another embodiment comprises cyclosporin A, Promulgen G®, and Caraway oil.

Another embodiment comprises cyclosporin A, Renex 30®, and Caraway oil.

Another embodiment comprises cyclosporin A, Renex 36®, and Caraway oil.

Another embodiment comprises cyclosporin A, Rhodasun A 24®, and Caraway oil.

Another embodiment comprises cyclosporin A, Rhodasurf AAE-10®, and Caraway oil.

Another embodiment comprises cyclosporin A, Rhodasurf BEH-25®, and Caraway oil.

Another embodiment comprises cyclosporin A, Rhodasurf BEH-40®, and Caraway oil.

Another embodiment comprises cyclosporin A, Rhodasurf DA 530®, and Caraway oil.

Another embodiment comprises cyclosporin A, Rhodasurf DA 630®, and Caraway oil.

Another embodiment comprises cyclosporin A, Rhodasurf DA 639®, and Caraway oil.

Another embodiment comprises cyclosporin A, Rhodasurf LAN-23®, and Caraway oil.

Another embodiment comprises cyclosporin A, Rhodasurf ON-870®, and Caraway oil.

Another embodiment comprises cyclosporin A, Rhodasurf ON-877®, and Caraway oil.

Another embodiment comprises cyclosporin A, Rhodasurf TB-970 FLK®, and Caraway oil.

Another embodiment comprises cyclosporin A, Ritacet-20®, and Caraway oil.

Another embodiment comprises cyclosporin A, Ritachol 1000®, and Caraway oil.

Another embodiment comprises cyclosporin A, Ritachol 2000®, and Caraway oil.

Another embodiment comprises cyclosporin A, Ritachol 5000®, and Caraway oil.

Another embodiment comprises cyclosporin A, Ritox 35®, and Caraway oil.

Another embodiment comprises cyclosporin A, Surfonic DA-4®, and Caraway oil.

Another embodiment comprises cyclosporin A, Surfonic DA-6®, and Caraway oil.

Another embodiment comprises cyclosporin A, Surfonic L46-7®, and Caraway oil.

Another embodiment comprises cyclosporin A, Surfonic POA®, and Caraway oil.

Another embodiment comprises cyclosporin A, Synthrapol KB®, and Caraway oil.

Another embodiment comprises cyclosporin A, Teginacid®, and Caraway oil.

Another embodiment comprises cyclosporin A, Teginacid C®, and Caraway oil.

Another embodiment comprises cyclosporin A, Tegotens EC 11®, and Caraway oil.

Another embodiment comprises cyclosporin A, Tinegal NA®, and Caraway oil.

Another embodiment comprises cyclosporin A, Tomadol 400®, and Caraway oil.

Another embodiment comprises cyclosporin A, Tomadol 600®, and Caraway oil.

Another embodiment comprises cyclosporin A, Tomadol 900®, and Caraway oil.

Another embodiment comprises cyclosporin A, Uniperol O®, and Caraway oil.

Another embodiment comprises cyclosporin A, Witconol SN Series®, and Caraway oil.

Another embodiment comprises cyclosporin A, an Ethoxylated Alkylphenol, and Caraway oil.

Another embodiment comprises cyclosporin A, Antarox LF-222®, and Caraway oil.

Another embodiment comprises cyclosporin A, Atlox 775®, and Caraway oil.

Another embodiment comprises cyclosporin A, Caloxylate N-9®, and Caraway oil.

Another embodiment comprises cyclosporin A, Canasol NF-1000®, and Caraway oil.

Another embodiment comprises cyclosporin A, Canasol NF-3000®, and Caraway oil.

Another embodiment comprises cyclosporin A, Canasol NF-3070®, and Caraway oil.

Another embodiment comprises cyclosporin A, Canasol OF 1670®, and Caraway oil.

Another embodiment comprises cyclosporin A, Canasol OF 2570®, and Caraway oil.

Another embodiment comprises cyclosporin A, Canasol OF 4070®, and Caraway oil.

Another embodiment comprises cyclosporin A, Chemax DNP-8®, and Caraway oil.

Another embodiment comprises cyclosporin A, Chemax DNP-18®, and Caraway oil.

Another embodiment comprises cyclosporin A, Chemax DNP-150/50®, and Caraway oil.

Another embodiment comprises cyclosporin A, DeSonic 1.5N®, and Caraway oil.

Another embodiment comprises cyclosporin A, DeSonic 4N®, and Caraway oil.

Another embodiment comprises cyclosporin A, DeSonic 5N®, and Caraway oil.

Another embodiment comprises cyclosporin A, DeSonic 6D®, and Caraway oil.

Another embodiment comprises cyclosporin A, DeSonic 6N®, and Caraway oil.

Another embodiment comprises cyclosporin A, DeSonic 7N®, and Caraway oil.

Another embodiment comprises cyclosporin A, DeSonic 9N®, and Caraway oil.

Another embodiment comprises cyclosporin A, DeSonic 10D®, and Caraway oil.

Another embodiment comprises cyclosporin A, DeSonic 11N®, and Caraway oil.

Another embodiment comprises cyclosporin A, DeSonic 12N®, and Caraway oil.

Another embodiment comprises cyclosporin A, DeSonic 15N®, and Caraway oil.

Another embodiment comprises cyclosporin A, DeSonic 20N®, and Caraway oil.

Another embodiment comprises cyclosporin A, Eccoscour RC®, and Caraway oil.

Another embodiment comprises cyclosporin A, Eccoterge EO-100®, and Caraway oil.

Another embodiment comprises cyclosporin A, Emulsifier 632/90%®, and Caraway oil.

Another embodiment comprises cyclosporin A, Geronol AG-821®, and Caraway oil.

Another embodiment comprises cyclosporin A, Gradonic N-95®, and Caraway oil.

Another embodiment comprises cyclosporin A, Hetoxide NP-4®, and Caraway oil.

Another embodiment comprises cyclosporin A, Hetoxide NP-30®, and Caraway oil.

Another embodiment comprises cyclosporin A, Hostapal N-100®, and Caraway oil.

Another embodiment comprises cyclosporin A, Hostapal N-110®, and Caraway oil.

Another embodiment comprises cyclosporin A, Igepal CTA-639W®, and Caraway oil.

Another embodiment comprises cyclosporin A, Igepal DAP-9®, and Caraway oil.

Another embodiment comprises cyclosporin A, Igepal OD-410®, and Caraway oil.

Another embodiment comprises cyclosporin A, Igepal SS-837®, and Caraway oil.

Another embodiment comprises cyclosporin A, Lipocol NP-9 USP®, and Caraway oil.

Another embodiment comprises cyclosporin A, Macol DNP-10®, and Caraway oil.

Another embodiment comprises cyclosporin A, Marlophen NP 5®, and Caraway oil.

Another embodiment comprises cyclosporin A, Marlophen P 1®, and Caraway oil.

Another embodiment comprises cyclosporin A, Surfonic NB®, and Caraway oil.

Another embodiment comprises cyclosporin A, Surfonic OPB-307®, and Caraway oil.

Another embodiment comprises cyclosporin A, Surfonic OPB-407®, and Caraway oil.

Another embodiment comprises cyclosporin A, Syn Fac 334®, and Caraway oil.

Another embodiment comprises cyclosporin A, Syn Fac 8216®, and Caraway oil.

Another embodiment comprises cyclosporin A, Triton N-57®, and Caraway oil.

Another embodiment comprises cyclosporin A, Trycol 6956®, and Caraway oil.

Another embodiment comprises cyclosporin A, Trycol 6961®, and Caraway oil.

Another embodiment comprises cyclosporin A, Trycol 6964®, and Caraway oil.

Another embodiment comprises cyclosporin A, Trycol 6969®, and Caraway oil.

Another embodiment comprises cyclosporin A, Trycol 6974®, and Caraway oil.

Another embodiment comprises cyclosporin A, Witbreak DRB-127®, and Caraway oil.

Another embodiment comprises cyclosporin A, Witbreak DRB-127®, and Caraway oil.

Another embodiment comprises cyclosporih A, Witconol NP Series®, and Caraway oil.

Another embodiment comprises cyclosporin A, an Ethoxylated Aryl Phenol, and Caraway oil.

Another embodiment comprises cyclosporin A, Soprophor BSU®, and Caraway oil.

Another embodiment comprises cyclosporin A, Soprophor CY/8®, and Caraway oil.

Another embodiment comprises cyclosporin A, Soprophor S/25®, and Caraway oil.

Another embodiment comprises cyclosporin A, Witconol NIO®, and Caraway oil.

Another embodiment comprises cyclosporin A, Witconol NIW®, and Caraway oil.

Another embodiment comprises cyclosporin A, Witconol S-100®, and Caraway oil.

Another embodiment comprises cyclosporin A, an Ethoxylated Fatty Acid, and Caraway oil.

Another embodiment comprises cyclosporin A, Aldo PGHMS®, and Caraway oil.

Another embodiment comprises cyclosporin A, Alkamuls TO-15/HR®, and Caraway oil.

Another embodiment comprises cyclosporin A, Armotan AL-69-66®, and Caraway oil.

Another embodiment comprises cyclosporin A, Cerasynt 840®, and Caraway oil.

Another embodiment comprises cyclosporin A, Cerasynt 945®, and Caraway oil.

Another embodiment comprises cyclosporin A, Crystal Inhibitor No. 5®, and Caraway oil.

Another embodiment comprises cyclosporin A, DeThox Acid L-9®, and Caraway oil.

Another embodiment comprises cyclosporin A, DeThox Acid S-8®, and Caraway oil.

Another embodiment comprises cyclosporin A, Ethofat 242/25®, and Caraway oil.

Another embodiment comprises cyclosporin A, Hydropalat 65®, and Caraway oil.

Another embodiment comprises cyclosporin A, Lipo EGMS®, and Caraway oil.

Another embodiment comprises cyclosporin A, Lipopeg 2 DL®, and Caraway oil.

Another embodiment comprises cyclosporin A, Lipopeg 4 DL®, and Caraway oil.

Another embodiment comprises cyclosporin A, Lipopeg 4-L®, and Caraway oil.

Another embodiment comprises cyclosporin A, Lipopeg 39-S®, and Caraway oil.

Another embodiment comprises cyclosporin A, Lipopeg 4-S®, and Caraway oil.

Another embodiment comprises cyclosporin A, Lipopeg 10-S®, and Caraway oil.

Another embodiment comprises cyclosporin A, Lipopeg 100-S®, and Caraway oil.

Another embodiment comprises cyclosporin A, Lipopeg 6000 DS®, and Caraway oil.

Another embodiment comprises cyclosporin A, Lumulse 40-L®, and Caraway oil.

Another embodiment comprises cyclosporin A, Lumulse 40-S®, and Caraway oil.

Another embodiment comprises cyclosporin A, Lumulse 42-L®, and Caraway oil.

Another embodiment comprises cyclosporin A, Lumulse 42-S®, and Caraway oil.

Another embodiment comprises cyclosporin A, Lumulse 100-S®, and Caraway oil.

Another embodiment comprises cyclosporin A, Lumulse 602-S®, and Caraway oil.

Another embodiment comprises cyclosporin A, Magrabar PGE-20-0®, and Caraway oil.

Another embodiment comprises cyclosporin A, Magrabar PGE-20L®, and Caraway oil.

Another embodiment comprises cyclosporin A, Magrabar PGE-20T®, and Caraway oil.

Another embodiment comprises cyclosporin A, Magrabar PGE-22-0®, and Caraway oil.

Another embodiment comprises cyclosporin A, Magrabar PGE-22L®, and Caraway oil.

Another embodiment comprises cyclosporin A, Magrabar PGE-22T®, and Caraway oil.

Another embodiment comprises cyclosporin A, Magrabar PGE-40-0®, and Caraway oil.

Another embodiment comprises cyclosporin A, Magrabar PGE-40L®, and Caraway oil.

Another embodiment comprises cyclosporin A, Magrabar PGE-40T®, and Caraway oil.

Another embodiment comprises cyclosporin A, Magrabar PGE-42-0®, and Caraway oil.

Another embodiment comprises cyclosporin A, Magrabar PGE-42L®, and Caraway oil.

Another embodiment comprises cyclosporin A, Magrabar PGE-42T®, and Caraway oil.

Another embodiment comprises cyclosporin A, Magrabar PGE-60-0®, and Caraway oil.

Another embodiment comprises cyclosporin A, Magrabar PGE-60L®, and Caraway oil.

Another embodiment comprises cyclosporin A, Magrabar PGE-60T®, and Caraway oil.

Another embodiment comprises cyclosporin A, Magrabar PGE-62-0®, and Caraway oil.

Another embodiment comprises cyclosporin A, Magrabar PGE-62L®, and Caraway oil.

Another embodiment comprises cyclosporin A, Magrabar PGE-62T®, and Caraway oil.

Another embodiment comprises cyclosporin A, Mapeg S-40K®, and Caraway oil.

Another embodiment comprises cyclosporin A, Marlowet OTS®, and Caraway oil.

Another embodiment comprises cyclosporin A, Naturechem PGR®, and Caraway oil.

Another embodiment comprises cyclosporin A, PG No. 4®, and Caraway oil.

Another embodiment comprises cyclosporin A, Renex 20®, and Caraway oil.

Another embodiment comprises cyclosporin A, Ritox 52®, and Caraway oil.

Another embodiment comprises cyclosporin A, Ritox 53®, and Caraway oil.

Another embodiment comprises cyclosporin A, Ritox 59®, and Caraway oil.

Another embodiment comprises cyclosporin A, Surax 8916/A®, and Caraway oil.

Another embodiment comprises cyclosporin A, Tego Acid S 40 P®, and Caraway oil.

Another embodiment comprises cyclosporin A, Tego Acid S 100 P®, and Caraway oil.

Another embodiment comprises cyclosporin A, Tween 20®, and Caraway oil.

Another embodiment comprises cyclosporin A, Volpo 131®, and Caraway oil.

Another embodiment comprises cyclosporin A, an Ethoxylated Fatty Ester or Oil, and Caraway oil.

Another embodiment comprises cyclosporin A, Acconon 6-C10®, and Caraway oil.

Another embodiment comprises cyclosporin A, Acconon CC-6®, and Caraway oil.

Another embodiment comprises cyclosporin A, Acconon CO-7®, and Caraway oil.

Another embodiment comprises cyclosporin A, Aldosperse 40/60 FG®, and Caraway oil.

Another embodiment comprises cyclosporin A, Aldosperse ML-23®, and Caraway oil.

Another embodiment comprises cyclosporin A, Aldosperse MS-20 FG®, and Caraway oil.

Another embodiment comprises cyclosporin A, Alkamuls EL-620®, and Caraway oil.

Another embodiment comprises cyclosporin A, Alkamuls EL-71 9®, and Caraway oil.

Another embodiment comprises cyclosporin A, Alkamuls EL-985®, and Caraway oil.

Another embodiment comprises cyclosporin A, Arlatone G®, and Caraway oil.

Another embodiment comprises cyclosporin A, Arlatone T®, and Caraway oil.

Another embodiment comprises cyclosporin A, Atlas G-1045A®, and Caraway oil,

Another embodiment comprises cyclosporin A, Atlas G-1086®, and Caraway oil.

Another embodiment comprises cyclosporin A, Atlas G-1087®, and Caraway oil.

Another embodiment comprises cyclosporin A, Atlas G-1089®, and Caraway oil.

Another embodiment comprises cyclosporin A, Atlas G-1096®, and Caraway oil.

Another embodiment comprises cyclosporin A, Atlas G-1292®, and Caraway oil.

Another embodiment comprises cyclosporin A, Atlas G-1293®, and Caraway oil.

Another embodiment comprises cyclosporin A, Atlas G-1300®, and Caraway oil.

Another embodiment comprises cyclosporin A, Atlas G-7076®, and Caraway oil.

Another embodiment comprises cyclosporin A, Capmul EMG®, and Caraway oil.

Another embodiment comprises cyclosporin A, Chemonic CO-40®, and Caraway oil.

Another embodiment comprises cyclosporin A, Chemonic LI-3®, and Caraway oil.

Another embodiment comprises cyclosporin A, Chemonic LI-7®, and Caraway oil.

Another embodiment comprises cyclosporin A, Cirrasol GM®, and Caraway oil.

Another embodiment comprises cyclosporin A, Cremophor CO 40®, and Caraway oil.

Another embodiment comprises cyclosporin A, Cremophor CO410®, and Caraway oil.

Another embodiment comprises cyclosporin A, Cremophor EL®, and Caraway oil.

Another embodiment comprises cyclosporin A, Cremophor GC7®, and Caraway oil.

Another embodiment comprises cyclosporin A, Cremophor RH-40®, and Caraway oil.

Another embodiment comprises cyclosporin A, Crovol A-40®, and Caraway oil.

Another embodiment comprises cyclosporin A, Crovol A-70®, and Caraway oil.

Another embodiment comprises cyclosporin A, Crovol M-70®, and Caraway oil.

Another embodiment comprises cyclosporin A, Crovol PK-70®, and Caraway oil.

Another embodiment comprises cyclosporin A, Cutina E-24®, and Caraway oil.

Another embodiment comprises cyclosporin A, Dacospin 12-R®, and Caraway oil.

Another embodiment comprises cyclosporin A, Dehymuls HRE-7®, and Caraway oil.

Another embodiment comprises cyclosporin A, DeSonic 30C®, and Caraway oil.

Another embodiment comprises cyclosporin A, DeSonic 36C®, and Caraway oil.

Another embodiment comprises cyclosporin A, DeSonic 40C®, and Caraway oil.

Another embodiment comprises cyclosporin A, Durfax 60®, and Caraway oil.

Another embodiment comprises cyclosporin A, Durfax 65®, and Caraway oil.

Another embodiment comprises cyclosporin A, Durfax 80®, and Caraway oil.

Another embodiment comprises cyclosporin A, Durfax EOM®, and Caraway oil.

Another embodiment comprises cyclosporin A, Eccoterge NF-2®, and Caraway oil.

Another embodiment comprises cyclosporin A, Emulpon CO-360®, and Caraway oil.

Another embodiment comprises cyclosporin A, Emulpon CC-550®, and Caraway oil.

Another embodiment comprises cyclosporin A, Emulsogen EL®, and Caraway oil.

Another embodiment comprises cyclosporin A, Emulsogen HCO 040®, and Caraway oil.

Another embodiment comprises cyclosporin A, Emulsogen HCO 060®, and Caraway oil.

Another embodiment comprises cyclosporin A, Emulsynt 1055®, and Caraway oil.

Another embodiment comprises cyclosporin A, Ethox 3095®, and Caraway oil.

Another embodiment comprises cyclosporin A, Eumulgin RO-40®, and Caraway oil.

Another embodiment comprises cyclosporin A, Genapol G-260®, and Caraway oil.

Another embodiment comprises cyclosporin A, Glycosperse L-20®, and Caraway oil.

Another embodiment comprises cyclosporin A, Glycosperse O-5®, and Caraway oil.

Another embodiment comprises cyclosporin A, Glycosperse O-20®, and Caraway oil.

Another embodiment comprises cyclosporin A, Glycosperse O-20 FG®, and Caraway oil.

Another embodiment comprises cyclosporin A, Glycosperse S-20®, and Caraway oil.

Another embodiment comprises cyclosporin A, Glycosperse S-20 FG®, and Caraway oil.

Another embodiment comprises cyclosporin A, Glycosperse TS-20®, and Caraway oil.

Another embodiment comprises cyclosporin A, Glycosperse TS-20 FG®, and Caraway oil.

Another embodiment comprises cyclosporin A, Hetan SL®, and Caraway oil.

Another embodiment comprises cyclosporin A, Hetan SO®, and Caraway oil.

Another embodiment comprises cyclosporin A, Hetan SS®, and Caraway oil.

Another embodiment comprises cyclosporin A, Hetoxide C-2®, and Caraway oil.

Another embodiment comprises cyclosporin A, Hetoxide C-9®, and Caraway oil.

Another embodiment comprises cyclosporin A, Hetoxide C-15®, and Caraway oil.

Another embodiment comprises cyclosporin A, Hetoxide C-25®, and Caraway oil.

Another embodiment comprises cyclosporin A, Hetoxide C-40®, and Caraway oil.

Another embodiment comprises cyclosporin A, Hetoxide C-200®, and Caraway oil.

Another embodiment comprises cyclosporin A, Hetoxide C-200-50%®, and Caraway oil.

Another embodiment comprises cyclosporin A, Hetoxide GC-30®, and Caraway oil.

Another embodiment comprises cyclosporin A, Hetoxide HC-60®, and Caraway oil.

Another embodiment comprises cyclosporin A, Ice No. 2®, and Caraway oil.

Another embodiment comprises cyclosporin A, Incrocas 30/40®, and Caraway oil.

Another embodiment comprises cyclosporin A, Lexol EC®, and Caraway oil.

Another embodiment comprises cyclosporin A, Lexol EO®, and Caraway oil.

Another embodiment comprises cyclosporin A, Lipocol HCO-40®, and Caraway oil.

Another embodiment comprises cyclosporin A, Lipocol HCO-60®, and Caraway oil.

Another embodiment comprises cyclosporin A, Lipocol O-3 Special®, and Caraway oil.

Another embodiment comprises cyclosporin A, Lipopeg 2-L®, and Caraway oil.

Another embodiment comprises cyclosporin A, Lipopeg 4-DO®, and Caraway oil.

Another embodiment comprises cyclosporin A, Lipopeg 4-DS®, and Caraway oil.

Another embodiment comprises cyclosporin A, Lipovol GTB®, and Caraway oil.

Another embodiment comprises cyclosporin A, Lonzest SML-20®, and Caraway oil.

Another embodiment comprises cyclosporin A, Lonzest SMO-20®, and Caraway oil.

Another embodiment comprises cyclosporin A, Lonzest SMS-20®, and Caraway oil.

Another embodiment comprises cyclosporin A, Lonzest STO-20®, and Caraway oil.

Another embodiment comprises cyclosporin A, Lonzest STS-20®, and Caraway oil.

Another embodiment comprises cyclosporin A, Lumulse GR-40®, and Caraway oil.

Another embodiment comprises cyclosporin A, Lumulse GRH-40®, and Caraway oil.

Another embodiment comprises cyclosporin A, Lumulse POE (7) GML®, and Caraway oil.

Another embodiment comprises cyclosporin A, Lumulse POE (12) Glyc®, and Caraway oil.

Another embodiment comprises cyclosporin A, Lumulse POE (40) MS KP®, and Caraway oil.

Another embodiment comprises cyclosporin A, Marlowet 4750®, and Caraway oil.

Another embodiment comprises cyclosporin A, Marlowet LVS®, and Caraway oil.

Another embodiment comprises cyclosporin A, Marlowet R 11®, and Caraway oil.

Another embodiment comprises cyclosporin A, Marlowet R 40, and Caraway oil.

Another embodiment comprises cyclosporin A, Mazol 80 MGK®, and Caraway oil.

Another embodiment comprises cyclosporin A, Nonionic Emulsifier T-9®, and Caraway oil.

Another embodiment comprises cyclosporin A, Oronal LCG®, and Caraway oil.

Another embodiment comprises cyclosporin A, Polyderm PPI-CO-200®, and Caraway oil.

Another embodiment comprises cyclosporin A, Polyderm PPI-CO-40®, and Caraway oil.

Another embodiment comprises cyclosporin A, Rewoderm LI 520-70®, and Caraway oil.

Another embodiment comprises cyclosporin A, Ritapeg 150 DS®, and Caraway oil.

Another embodiment comprises cyclosporin A, Softigen 767®, and Caraway oil.

Another embodiment comprises cyclosporin A, Surfactol 318®, and Caraway oil.

Another embodiment comprises cyclosporin A, Surfactol 365®, and Caraway oil.

Another embodiment comprises cyclosporin A, Syn Lube 107®, and Caraway oil.

Another embodiment comprises cyclosporin A, Syn Lube 728®, and Caraway oil.

Another embodiment comprises cyclosporin A, Syn Lube 1632H®, and Caraway oil.

Another embodiment comprises cyclosporin A, Syn Lube 6277-A®, and Caraway oil.

Another embodiment comprises cyclosporin A, T-Det C-20®, and Caraway oil.

Another embodiment comprises cyclosporin A, T-Det C-40®, and Caraway oil.

Another embodiment comprises cyclosporin A, Tally 100 Plus®, and Caraway oil.

Another embodiment comprises cyclosporin A, Uniperol EL®, and Caraway oil.

Another embodiment comprises cyclosporin A, a Fatty Ester, and Caraway oil.

Another embodiment comprises cyclosporin A, Actralube-Syn 147®, and Caraway oil.

Another embodiment comprises cyclosporin A, Atlas G-1556®, and Caraway oil.

Another embodiment comprises cyclosporin A, Atlas G-1564®, and Caraway oil.

Another embodiment comprises cyclosporin A, Atlasol Base Oil S®, and Caraway oil.

Another embodiment comprises cyclosporin A, Base ML®, and Caraway oil.

Another embodiment comprises cyclosporin A, Base MT®, and Caraway oil.

Another embodiment comprises cyclosporin A, Cerasynt 303®, and Caraway oil.

Another embodiment comprises cyclosporin A, Dermol 1012®, and Caraway oil.

Another embodiment comprises cyclosporin A, Kemester 4000®, and Caraway oil.

Another embodiment comprises cyclosporin A, Lactipol S®, and Caraway oil.

Another embodiment comprises cyclosporin A, Magrabar PGO®, and Caraway oil.

Another embodiment comprises cyclosporin A, Mayco Base BFO®, and Caraway oil.

Another embodiment comprises cyclosporin A, Methyl Linoleate®, and Caraway oil.

Another embodiment comprises cyclosporin A, Pationic 122A®, and Caraway oil.

Another embodiment comprises cyclosporin A, Pationic 138C®, and Caraway oil.

Another embodiment comprises cyclosporin A, Pationic CSL®, and Caraway oil.

Another embodiment comprises cyclosporin A, Pationic ISL®, and Caraway oil.

Another embodiment comprises cyclosporin A, Pationic SBL®, and Caraway oil.

Another embodiment comprises cyclosporin A, Pationic SSL®, and Caraway oil.

Another embodiment comprises cyclosporin A, Ritasol®, and Caraway oil.

Another embodiment comprises cyclosporin A, Tego Alkanol CS 20®, and Caraway oil.

Another embodiment comprises cyclosporin A, Tego Alkanol L23 P®, and Caraway oil.

Another embodiment comprises cyclosporin A, Tego Alkanol S2®, and Caraway oil.

Another embodiment comprises cyclosporin A, Tego Alkanol S20 P®, and Caraway oil.

Another embodiment comprises cyclosporin A, Triemulsifier 600 MS®, and Caraway oil.

Another embodiment comprises cyclosporin A, a Glycerol Esters, and Caraway oil.

Another embodiment comprises cyclosporin A, Agro #9 Wint SBO®, and Caraway oil.

Another embodiment comprises cyclosporin A, Ahcovel Base 700®, and Caraway oil.

Another embodiment comprises cyclosporin A, Aldo HMS FG®, and Caraway oil.

Another embodiment comprises cyclosporin A, Aldo MLD®, and Caraway oil.

Another embodiment comprises cyclosporin A, Aldo MLD FG®, and Caraway oil.

Another embodiment comprises cyclosporin A, Aldo MO FG®, and Caraway oil.

Another embodiment comprises cyclosporin A, Aldo MS®, and Caraway oil.

Another embodiment comprises cyclosporin A, Aldo MS FG®, and Caraway oil.

Another embodiment comprises cyclosporin A, Aldo MS LG FG®, and Caraway oil.

Another embodiment comprises cyclosporin A, Aldo MSD®, and Caraway oil.

Another embodiment comprises cyclosporin A, Aldo MSD FG®, and Caraway oil.

Another embodiment comprises cyclosporin A, Aldosperse O-20 FG®, and Caraway oil.

Another embodiment comprises cyclosporin A, Aldosperse TS-20 FG®, and Caraway oil.

Another embodiment comprises cyclosporin A, Aldosperse TS-40 FG®, and Caraway oil.

Another embodiment comprises cyclosporin A, Arlacel 165®, and Caraway oil.

Another embodiment comprises cyclosporin A, Arlacel 186®, and Caraway oil.

Another embodiment comprises cyclosporin A, Capmul GMO®, and Caraway oil.

Another embodiment comprises cyclosporin A, Capmul GMS®, and Caraway oil.

Another embodiment comprises cyclosporin A, Caprol 3GO®, and Caraway oil.

Another embodiment comprises cyclosporin A, Caprol 3GVS®, and Caraway oil.

Another embodiment comprises cyclosporin A, Caprol 6G2S®, and Caraway oil.

Another embodiment comprises cyclosporin A, Caprol 10G40®, and Caraway oil.

Another embodiment comprises cyclosporin A, Caprol 10G100®, and Caraway oil.

Another embodiment comprises cyclosporin A, Caprol ET®, and Caraway oil.

Another embodiment comprises cyclosporin A, Caprol PGE860®, and Caraway oil.

Another embodiment comprises cyclosporin A, Cerasynt 945®, and Caraway oil.

Another embodiment comprises cyclosporin A, Cerasynt GMS®, and Caraway oil.

Another embodiment comprises cyclosporin A, Cerasynt Q®, and Caraway oil.

Another embodiment comprises cyclosporin A, Cerasynt SD®, and Caraway oil.

Another embodiment comprises cyclosporin A, Cerasynt WM®, and Caraway oil.

Another embodiment comprises cyclosporin A, Chemsperse 14®, and Caraway oil.

Another embodiment comprises cyclosporin A, Cremophor GO-32®, and Caraway oil.

Another embodiment comprises cyclosporin A, Cremophor GS11®, and Caraway oil.

Another embodiment comprises cyclosporin A, Cremophor GS-32®, and Caraway oil.

Another embodiment comprises cyclosporin A, Cutina KD-16®, and Caraway oil.

Another embodiment comprises cyclosporin A, Dehymuls PGPH®, and Caraway oil.

Another embodiment comprises cyclosporin A, Dermol DGDIS®, and Caraway oil.

Another embodiment comprises cyclosporin A, Dermol DGMIS®, and Caraway oil.

Another embodiment comprises cyclosporin A, Dermol G-76®, and Caraway oil.

Another embodiment comprises cyclosporin A, Dermol G-7DI®, and Caraway oil.

Another embodiment comprises cyclosporin A, Dermol NGDI®, and Caraway oil.

Another embodiment comprises cyclosporin A, Dermolan GLH®, and Caraway oil.

Another embodiment comprises cyclosporin A, Drewmulse GMO®, and Caraway oil.

Another embodiment comprises cyclosporin A, Drewpol 3-5-M®, and Caraway oil.

Another embodiment comprises cyclosporin A, Durlac 100 W®, and Caraway oil.

Another embodiment comprises cyclosporin A, Dur-Lo®, and Caraway oil.

Another embodiment comprises cyclosporin A, Dynasan 118®, and Caraway oil.

Another embodiment comprises cyclosporin A, EC-25®, and Caraway oil.

Another embodiment comprises cyclosporin A, EM 40®, and Caraway oil.

Another embodiment comprises cyclosporin A, Emerest 2400®, and Caraway oil.

Another embodiment comprises cyclosporin A, Emerest 2452®, and Caraway oil.

Another embodiment comprises cyclosporin A, Empilan G-26®, and Caraway oil.

Another embodiment comprises cyclosporin A, Genapol TSM®, and Caraway oil.

Another embodiment comprises cyclosporin A, Hostacerin DGI®, and Caraway oil.

Another embodiment comprises cyclosporin A, Hostacerin DGL®, and Caraway oil.

Another embodiment comprises cyclosporin A, Hostacerin DGMS®, and Caraway oil.

Another embodiment comprises cyclosporin A, Hostacerin DGSB®, and Caraway oil.

Another embodiment comprises cyclosporin A, Ice No. 2®, and Caraway oil.

Another embodiment comprises cyclosporin A, Imwitor 742®, and Caraway oil.

Another embodiment comprises cyclosporin A, Imwitor 780 K®, and Caraway oil.

Another embodiment comprises cyclosporin A, Imwitor 960 Flakes®, and Caraway oil.

Another embodiment comprises cyclosporin A, Isolan GI 34®, and Caraway oil.

Another embodiment comprises cyclosporin A, Isolan GO 33®, and Caraway oil.

Another embodiment comprises cyclosporin A, Kemester 1000®, and Caraway oil.

Another embodiment comprises cyclosporin A, Kemester 2000®, and Caraway oil.

Another embodiment comprises cyclosporin A, Kemester 2000®, and Caraway oil.

Another embodiment comprises cyclosporin A, Kemester 6000SE®, and Caraway oil.

Another embodiment comprises cyclosporin A, Lamecreme DGE 18®, and Caraway oil.

Another embodiment comprises cyclosporin A, Lexemul 515®, and Caraway oil.

Another embodiment comprises cyclosporin A, Lexemul 561®, and Caraway oil.

Another embodiment comprises cyclosporin A, Lexemul AR®, and Caraway oil.

Another embodiment comprises cyclosporin A, Lexemul AS®, and Caraway oil.

Another embodiment comprises cyclosporin A, Lexemul GDL®, and Caraway oil.

Another embodiment comprises cyclosporin A, Lexemul T®, and Caraway oil.

Another embodiment comprises cyclosporin A, Lipomulse 165®, and Caraway oil.

Another embodiment comprises cyclosporin A, Lumulse GML K®, and Caraway oil.

Another embodiment comprises cyclosporin A, Lumulse GMO K®, and Caraway oil.

Another embodiment comprises cyclosporin A, Lumulse GMR K®, and Caraway oil.

Another embodiment comprises cyclosporin A, Lumulse GMT K®, and Caraway oil.

Another embodiment comprises cyclosporin A, Magrabar GMC®, and Caraway oil.

Another embodiment comprises cyclosporin A, Magrabar GMO-CK®, and Caraway oil.

Another embodiment comprises cyclosporin A, Magrabar MDG-5050®, and Caraway oil.

Another embodiment comprises cyclosporin A, Magrabar PGO-315®, and Caraway oil.

Another embodiment comprises cyclosporin A, Magrabar PGO-1010®, and Caraway oil.

Another embodiment comprises cyclosporin A, Mazol 300K®, and Caraway oil.

Another embodiment comprises cyclosporin A, Mazol GMO-K®, and Caraway oil.

Another embodiment comprises cyclosporin A, Mazol GMS-K®, and Caraway oil.

Another embodiment comprises cyclosporin A, Mazol PG031-K®, and Caraway oil.

Another embodiment comprises cyclosporin A, Miglyol 812®, and Caraway oil.

Another embodiment comprises cyclosporin A, Norfox 165C®, and Caraway oil.

Another embodiment comprises cyclosporin A, Schercemol GMIS®, and Caraway oil.

Another embodiment comprises cyclosporin A, Tegin®, and Caraway oil.

Another embodiment comprises cyclosporin A, Tegin 4100 Pellets®, and Caraway oil.

Another embodiment comprises cyclosporin A, Tegin M Pellets®, and Caraway oil.

Another embodiment comprises cyclosporin A, Tegin OV®, and Caraway oil.

Another embodiment comprises cyclosporin A, Teginacid H®, and Caraway oil.

Another embodiment comprises cyclosporin A, Tego Cosmo P813®, and Caraway oil.

Another embodiment comprises cyclosporin A, Wickenol 535®, and Caraway oil.

Another embodiment comprises cyclosporin A, Witconol 14®, and Caraway oil.

Another embodiment comprises cyclosporin A, Witconol 14®, and Caraway oil.

Another embodiment comprises cyclosporin A, Witconol 14F®, and Caraway oil.

Another embodiment comprises cyclosporin A, Witconol 18L®, and Caraway oil.

Another embodiment comprises cyclosporin A, Witconol GOT®, and Caraway oil.

Another embodiment comprises cyclosporin A, Witconol MST®, and Caraway oil.

Another embodiment comprises cyclosporin A, Witconol RHT®, and Caraway oil.

Another embodiment comprises cyclosporin A, a Glycol Ester, and Caraway oil.

Another embodiment comprises cyclosporin A, Alkamuls 600 DO®, and Caraway oil.

Another embodiment comprises cyclosporin A, Alkamuls SEG®, and Caraway oil.

Another embodiment comprises cyclosporin A, Atlas EM-2®, and Caraway oil.

Another embodiment comprises cyclosporin A, Cerasynt IP®, and Caraway oil.

Another embodiment comprises cyclosporin A, Cerasynt M®, and Caraway oil.

Another embodiment comprises cyclosporin A, Cerasynt MN®, and Caraway oil.

Another embodiment comprises cyclosporin A, Cerasynt PA®, and Caraway oil.

Another embodiment comprises cyclosporin A, Chemsperse EGDS®, and Caraway oil.

Another embodiment comprises cyclosporin A, Chemsperse EGMS®, and Caraway oil.

Another embodiment comprises cyclosporin A, Colonial Monolaurin®, and Caraway oil.

Another embodiment comprises cyclosporin A, DeMuls SGE-95®, and Caraway oil.

Another embodiment comprises cyclosporin A, Eccoterge 200®, and Caraway oil.

Another embodiment comprises cyclosporin A, Emerest 2380®, and Caraway oil.

Another embodiment comprises cyclosporin A, Ethox 2610®, and Caraway oil.

Another embodiment comprises cyclosporin A, Ethox DO-9®, and Caraway oil.

Another embodiment comprises cyclosporin A, Ethox DO-14®, and Caraway oil.

Another embodiment comprises cyclosporin A, Ethox SO-9®, and Caraway oil.

Another embodiment comprises cyclosporin A, Fizul MD-318®, and Caraway oil.

Another embodiment comprises cyclosporin A, Genapol EGDS-VHP®, and Caraway oil.

Another embodiment comprises cyclosporin A, Genapol TS Powder®, and Caraway oil.

Another embodiment comprises cyclosporin A, Hostacerin WO®, and Caraway oil.

Another embodiment comprises cyclosporin A, Inversol 140®, and Caraway oil.

Another embodiment comprises cyclosporin A, Kemester 104®, and Caraway oil.

Another embodiment comprises cyclosporin A, Kemester 205®, and Caraway oil.

Another embodiment comprises cyclosporin A, Kemester 226®, and Caraway oil.

Another embodiment comprises cyclosporin A, Kemester 5221SE®, and Caraway oil.

Another embodiment comprises cyclosporin A, Kemester EGDS®, and Caraway oil.

Another embodiment comprises cyclosporin A, Lexemul EGDS®, and Caraway oil.

Another embodiment comprises cyclosporin A, Lexemul EGMS®, and Caraway oil.

Another embodiment comprises cyclosporin A, Lexemul P®, and Caraway oil.

Another embodiment comprises cyclosporin A, Lipo DGLS®, and Caraway oil.

Another embodiment comprises cyclosporin A, Lipo EGDS®, and Caraway oil.

Another embodiment comprises cyclosporin A, Lipo PGMS®, and Caraway oil.

Another embodiment comprises cyclosporin A, Liposorb S-4®, and Caraway oil.

Another embodiment comprises cyclosporin A, Liposorb TO-20®, and Caraway oil.

Another embodiment comprises cyclosporin A, Lumulse PGO®, and Caraway oil.

Another embodiment comprises cyclosporin A, Mackester EGDS®, and Caraway oil.

Another embodiment comprises cyclosporin A, Mackester EGMS®, and Caraway oil.

Another embodiment comprises cyclosporin A, Mackester GSTP®, and Caraway oil.

Another embodiment comprises cyclosporin A, Mackester Series®, and Caraway oil.

Another embodiment comprises cyclosporin A, Magrabar PDG-50®, and Caraway oil.

Another embodiment comprises cyclosporin A, Mapeg 6000 DS®, and Caraway oil.

Another embodiment comprises cyclosporin A, Marlowet 4702®, and Caraway oil.

Another embodiment comprises cyclosporin A, Monalube 305®, and Caraway oil.

Another embodiment comprises cyclosporin A, Monalube 310®, and Caraway oil.

Another embodiment comprises cyclosporin A, Monalube 315®, and Caraway oil.

Another embodiment comprises cyclosporin A, Monalube 320®, and Caraway oil.

Another embodiment comprises cyclosporin A, Monalube 325®, and Caraway oil.

Another embodiment comprises cyclosporin A, Monalube 330®, and Caraway oil.

Another embodiment comprises cyclosporin A, Naturechem PGHS®, and Caraway oil.

Another embodiment comprises cyclosporin A, Polycastorol PLO-840®, and Caraway oil.

Another embodiment comprises cyclosporin A, Polytex 10OM®, and Caraway oil.

Another embodiment comprises cyclosporin A, Ritasynt IP®, and Caraway oil.

Another embodiment comprises cyclosporin A, Ross Chem PEG 600 DT®, and Caraway oil.

Another embodiment comprises cyclosporin A, Schercemol PGMS®, and Caraway oil.

Another embodiment comprises cyclosporin A, Sponto H-44C®, and Caraway oil.

Another embodiment comprises cyclosporin A, Tegin G®, and Caraway oil.

Another embodiment comprises cyclosporin A, Witbreak DGE-182®, and Caraway oil.

Another embodiment comprises cyclosporin A, Witbreak DGE-182®, and Caraway oil.

Another embodiment comprises cyclosporin A, Witbreak DRA-21®, and Caraway oil.

Another embodiment comprises cyclosporin A, Witbreak DRA-21®, and Caraway oil.

Another embodiment comprises cyclosporin A, Witbreak DRA-50®, and Caraway oil.

Another embodiment comprises cyclosporin A, Witbreak DRA-50®, and Caraway oil.

Another embodiment comprises cyclosporin A, Witconol F26-46®, and Caraway oil.

Another embodiment comprises cyclosporin A, Witconol H-32®, and Caraway oil.

Another embodiment comprises cyclosporin A, Witconol H-33®, and Caraway oil.

Another embodiment comprises cyclosporin A, Witconol H-35A®, and Caraway oil.

Another embodiment comprises cyclosporin A, Witconol RHP®, and Caraway oil.

Another embodiment comprises cyclosporin A, a Lanolin-based Derivative, and Caraway oil.

Another embodiment comprises cyclosporin A, Amerchol CAB®, and Caraway oil.

Another embodiment comprises cyclosporin A, Amerchol L-101®, and Caraway oil.

Another embodiment comprises cyclosporin A, Amerlate LFA-LO®, and Caraway oil.

Another embodiment comprises cyclosporin A, Amerlate P®, and Caraway oil.

Another embodiment comprises cyclosporin A, Barre Common Degras®, and Caraway oil.

Another embodiment comprises cyclosporin A, Cholesterol NF®, and Caraway oil.

Another embodiment comprises cyclosporin A, Crodalan AWS®, and Caraway oil.

Another embodiment comprises cyclosporin A, Crodalan LA®, and Caraway oil.

Another embodiment comprises cyclosporin A, Emery 1650®, and Caraway oil.

Another embodiment comprises cyclosporin A, Emery 1650®, and Caraway oil.

Another embodiment comprises cyclosporin A, Emery 1740®, and Caraway oil.

Another embodiment comprises cyclosporin A, Emery 1740®, and Caraway oil.

Another embodiment comprises cyclosporin A, Forlan 500®, and Caraway oil.

Another embodiment comprises cyclosporin A, Forlan L®, and Caraway oil.

Another embodiment comprises cyclosporin A, Laneto 50®, and Caraway oil.

Another embodiment comprises cyclosporin A, Laneto 100®, and Caraway oil.

Another embodiment comprises cyclosporin A, Laneto AWS®, and Caraway oil.

Another embodiment comprises cyclosporin A, Lanfrax 1776®, and Caraway oil.

Another embodiment comprises cyclosporin A, Lanfrax 1776®, and Caraway oil.

Another embodiment comprises cyclosporin A, Lanogel 21®, and Caraway oil.

Another embodiment comprises cyclosporin A, Lipolan®, and Caraway oil.

Another embodiment comprises cyclosporin A, Lipolan 31®, and Caraway oil.

Another embodiment comprises cyclosporin A, OHlan®, and Caraway oil.

Another embodiment comprises cyclosporin A, Polychol 5®, and Caraway oil.

Another embodiment comprises cyclosporin A, Polychol 15®, and Caraway oil.

Another embodiment comprises cyclosporin A, Ritacetyl®, and Caraway oil.

Another embodiment comprises cyclosporin A, Ritachol®, and Caraway oil.

Another embodiment comprises cyclosporin A, Ritahydrox®, and Caraway oil.

Another embodiment comprises cyclosporin A, Ritalafa®, and Caraway oil.

Another embodiment comprises cyclosporin A, Ritalan®, and Caraway oil.

Another embodiment comprises cyclosporin A, Ritalan AWS®, and Caraway oil.

Another embodiment comprises cyclosporin A, Ritalan C®, and Caraway oil.

Another embodiment comprises cyclosporin A, Ritawax®, and Caraway oil.

Another embodiment comprises cyclosporin A, Ritawax AEO®, and Caraway oil.

Another embodiment comprises cyclosporin A, Ritawax ALA®, and Caraway oil.

Another embodiment comprises cyclosporin A, Solan/Solan 50/Super Solan®, and Caraway oil.

Another embodiment comprises cyclosporin A, Super Hartolan/Hartolan®, and Caraway oil.

Another embodiment comprises cyclosporin A, Supersat AWS-4®, and Caraway oil.

Another embodiment comprises cyclosporin A, Supersat AWS-24®, and Caraway oil.

Another embodiment comprises cyclosporin A, a Lecithin or Lecithin Derivative, and Caraway oil.

Another embodiment comprises cyclosporin A, Alcolec®, and Caraway oil.

Another embodiment comprises cyclosporin A, Lecithin®, and Caraway oil.

Another embodiment comprises cyclosporin A, Lexin K®, and Caraway oil.

Another embodiment comprises cyclosporin A, Natipide®, and Caraway oil.

Another embodiment comprises cyclosporin A, a Lignin or Lignin Derivative, and Caraway oil.

Another embodiment comprises cyclosporin A, Diwatex XP 98®, and Caraway oil.

Another embodiment comprises cyclosporin A, Dynasperse LCD®, and Caraway oil.

Another embodiment comprises cyclosporin A, Indulin SAL®, and Caraway oil.

Another embodiment comprises cyclosporin A, Indulin W-1®, and Caraway oil.

Another embodiment comprises cyclosporin A, Indulin W-5®, and Caraway oil.

Another embodiment comprises cyclosporin A, Lignosol FTA®, and Caraway oil.

Another embodiment comprises cyclosporin A, Lignosol SFX-65®, and Caraway oil.

Another embodiment comprises cyclosporin A, Marasperse 52 CP®, and Caraway oil.

Another embodiment comprises cyclosporin A, Marasperse AG®, and Caraway oil.

Another embodiment comprises cyclosporin A, Marasperse CBOS-4®, and Caraway oil.

Another embodiment comprises cyclosporin A, Ufoxane 2®, and Caraway oil.

Another embodiment comprises cyclosporin A, a Methyl Ester, and Caraway oil.

Another embodiment comprises cyclosporin A, E.B. Cleaner AK®, and Caraway oil.

Another embodiment comprises cyclosporin A, Oleocal ME-70®, and Caraway oil.

Another embodiment comprises cyclosporin A, Trade Name Company®, and Caraway oil.

Another embodiment comprises cyclosporin A, Methyl Esters (cont'd)®, and Caraway oil.

Another embodiment comprises cyclosporin A, Oleocal ME-92®, and Caraway oil.

Another embodiment comprises cyclosporin A, Oleocal ME-112®, and Caraway oil.

Another embodiment comprises cyclosporin A, Oleocal ME-130®, and Caraway oil.

Another embodiment comprises cyclosporin A, a Monoglyceride or a Derivative thereof, and Caraway oil.

Another embodiment comprises cyclosporin A, Dynacet 211®, and Caraway oil.

Another embodiment comprises cyclosporin A, Hetsorb S-20®, and Caraway oil.

Another embodiment comprises cyclosporin A, Imwitor 191®, and Caraway oil.

Another embodiment comprises cyclosporin A, Imwitor 370®, and Caraway oil.

Another embodiment comprises cyclosporin A, lmwitor 375®, and Caraway oil.

Another embodiment comprises cyclosporin A, Imwitor 900®, and Caraway oil.

Another embodiment comprises cyclosporin A, Imwitor 945®, and Caraway oil.

Another embodiment comprises cyclosporin A, lmwitor 2020®, and Caraway oil.

Another embodiment comprises cyclosporin A, Kemester 5500®, and Caraway oil.

Another embodiment comprises cyclosporin A, Kemester 6000®, and Caraway oil.

Another embodiment comprises cyclosporin A, Magrabar GMC®, and Caraway oil.

Another embodiment comprises cyclosporin A, Magrabar GMO-CK®, and Caraway oil.

Another embodiment comprises cyclosporin A, Magrabar GPC-10®, and Caraway oil.

Another embodiment comprises cyclosporin A, Magrabar MDG-5050®, and Caraway oil.

Another embodiment comprises cyclosporin A, Monalube 335®, and Caraway oil.

Another embodiment comprises cyclosporin A, Monoglycerides Glyceryl Monestearate Archer Daniels Midland Company®, and Caraway oil.

Another embodiment comprises cyclosporin A, Rita GMS®, and Caraway oil.

Another embodiment comprises cyclosporin A, Ritamulse SCG®, and Caraway oil.

Another embodiment comprises cyclosporin A, Softigen 701®, and Caraway oil.

Another embodiment comprises cyclosporin A, Tally 100 Plus®, and Caraway oil.

Another embodiment comprises cyclosporin A, a Polyethylene Glycol, and Caraway oil.

Another embodiment comprises cyclosporin A, Emulgade PL 68/50®, and Caraway oil.

Another embodiment comprises cyclosporin A, Lumulse PEG®, and Caraway oil.

Another embodiment comprises cyclosporin A, Rhodasurf PEG-400®, and Caraway oil.

Another embodiment comprises cyclosporin A, Rhodasurf PEG-600®, and Caraway oil.

Another embodiment comprises cyclosporin A, Witconol PEG-400®, and Caraway oil.

Another embodiment comprises cyclosporin A, a Polymeric Surfactant, and Caraway oil.

Another embodiment comprises cyclosporin A, Acritamer PNC-EG®, and Caraway oil.

Another embodiment comprises cyclosporin A, Ag-Rho DEP-775®, and Caraway oil.

Another embodiment comprises cyclosporin A, APG 325N Glycoside®, and Caraway oil.

Another embodiment comprises cyclosporin A, Aristoflex AVC®, and Caraway oil.

Another embodiment comprises cyclosporin A, Aristoflex HMB®, and Caraway oil.

Another embodiment comprises cyclosporin A, Burco NPS-225®, and Caraway oil.

Another embodiment comprises cyclosporin A, Burco NPS-816®, and Caraway oil.

Another embodiment comprises cyclosporin A, Chemccinate 5603®, and Caraway oil.

Another embodiment comprises cyclosporin A, Cosmedia Guar C-261N®, and Caraway oil.

Another embodiment comprises cyclosporin A, Gantrez S-95®, and Caraway oil.

Another embodiment comprises cyclosporin A, Glucopon 220 UP®, and Caraway oil.

Another embodiment comprises cyclosporin A, Glucopon 225 DK®, and Caraway oil.

Another embodiment comprises cyclosporin A, Glucopon 425 N®, and Caraway oil.

Another embodiment comprises cyclosporin A, Glucopon 600 UP®, and Caraway oil.

Another embodiment comprises cyclosporin A, Glucopon 625 UP®, and Caraway oil.

Another embodiment comprises cyclosporin A, Pemulen 1621®, and Caraway oil.

Another embodiment comprises cyclosporin A, Pemulen 1622®, and Caraway oil.

Another embodiment comprises cyclosporin A, Pemulen TR-1®, and Caraway oil.

Another embodiment comprises cyclosporin A, Pemulen TR-2®, and Caraway oil.

Another embodiment comprises cyclosporin A, Plantacare 818®, and Caraway oil.

Another embodiment comprises cyclosporin A, Plantapon LGC Sorb®, and Caraway oil.

Another embodiment comprises cyclosporin A, Plantaren 1200N®, and Caraway oil.

Another embodiment comprises cyclosporin A, Plantaren 2000N®, and Caraway oil.

Another embodiment comprises cyclosporin A, Viscolam AT 64®, and Caraway oil.

Another embodiment comprises cyclosporin A, Viscolam AT 64P®, and Caraway oil.

Another embodiment comprises cyclosporin A, Viscolam AT 100®, and Caraway oil.

Another embodiment comprises cyclosporin A, Viscolam MAC 7®, and Caraway oil.

Another embodiment comprises cyclosporin A, Viscolam SMC 20®, and Caraway oil.

Another embodiment comprises cyclosporin A, Witbreak RTC-323®, and Caraway oil.

Another embodiment comprises cyclosporin A, WSI 3700®, and Caraway oil.

Another embodiment comprises cyclosporin A, a Propoxylated or Ethoxylated Fatty Acid, Alcohol, or Alkyl Phenol, and Caraway oil.

Another embodiment comprises cyclosporin A, Antarox AA-60®, and Caraway oil.

Another embodiment comprises cyclosporin A, Antarox LF-224®, and Caraway oil.

Another embodiment comprises cyclosporin A, Burcomul DFE-45®, and Caraway oil.

Another embodiment comprises cyclosporin A, Burcoterge LFE-1000®, and Caraway oil.

Another embodiment comprises cyclosporin A, Chemal LF-25B®, and Caraway oil.

Another embodiment comprises cyclosporin A, Chemal LF-40B®, and Caraway oil.

Another embodiment comprises cyclosporin A, Dehypon LS-36®, and Caraway oil.

Another embodiment comprises cyclosporin A, Dehypon LS-36®, and Caraway oil.

Another embodiment comprises cyclosporin A, Dehypon LS-54®, and Caraway oil.

Another embodiment comprises cyclosporin A, Dehypon LS-54®, and Caraway oil.

Another embodiment comprises cyclosporin A, Delonic 100 VLF®, and Caraway oil.

Another embodiment comprises cyclosporin A, Delonic LF-60 MOD®, and Caraway oil.

Another embodiment comprises cyclosporin A, Empiderm B®, and Caraway oil.

Another embodiment comprises cyclosporin A, Ethylan 1206®, and Caraway oil.

Another embodiment comprises cyclosporin A, Ethylan NS-500K®, and Caraway oil.

Another embodiment comprises cyclosporin A, Ethylan NS-500LQ®, and Caraway oil.

Another embodiment comprises cyclosporin A, Genapol 1392®, and Caraway oil.

Another embodiment comprises cyclosporin A, Genapol 2317®, and Caraway oil.

Another embodiment comprises cyclosporin A, Genapol 26EP710®, and Caraway oil.

Another embodiment comprises cyclosporin A, Genapol EP 1022®, and Caraway oil.

Another embodiment comprises cyclosporin A, Genapol EP 1024®, and Caraway oil.

Another embodiment comprises cyclosporin A, Genapol EP 6068®, and Caraway oil.

Another embodiment comprises cyclosporin A, Genapol NP915®, and Caraway oil.

Another embodiment comprises cyclosporin A, Kieralon MFB®, and Caraway oil.

Another embodiment comprises cyclosporin A, Lumisolve CSA-80 V®, and Caraway oil.

Another embodiment comprises cyclosporin A, Marlowet 5001®, and Caraway oil.

Another embodiment comprises cyclosporin A, Marlox FK 64®, and Caraway oil.

Another embodiment comprises cyclosporin A, Marlox MO 124®, and Caraway oil.

Another embodiment comprises cyclosporin A, Marlox S 58®, and Caraway oil.

Another embodiment comprises cyclosporin A, Nonatell 1003®, and Caraway oil.

Another embodiment comprises cyclosporin A, Nonatell 1038®, and Caraway oil.

Another embodiment comprises cyclosporin A, Nonatell 1052®, and Caraway oil.

Another embodiment comprises cyclosporin A, Nonatell 1061®, and Caraway oil.

Another embodiment comprises cyclosporin A, Nonatell 1075®, and Caraway oil.

Another embodiment comprises cyclosporin A, Nonatell 1088®, and Caraway oil.

Another embodiment comprises cyclosporin A, Nonatell 1123®, and Caraway oil.

Another embodiment comprises cyclosporin A, Nonatell 1153®, and Caraway oil.

Another embodiment comprises cyclosporin A, Nonatell 1161®, and Caraway oil.

Another embodiment comprises cyclosporin A, Nonatell 1172®, and Caraway oil.

Another embodiment comprises cyclosporin A, Nonatell 1181®, and Caraway oil.

Another embodiment comprises cyclosporin A, Norfox 36®, and Caraway oil.

Another embodiment comprises cyclosporin A, Procetyl AWS®, and Caraway oil.

Another embodiment comprises cyclosporin A, Sandoxylate SX 412®, and Caraway oil.

Another embodiment comprises cyclosporin A, Sandoxylate SX 418®, and Caraway oil.

Another embodiment comprises cyclosporin A, Surfonic JL-80X®, and Caraway oil.

Another embodiment comprises cyclosporin A, Surfonic JL-80X-B1®, and Caraway oil.

Another embodiment comprises cyclosporin A, Surfonic L4-29X®, and Caraway oil.

Another embodiment comprises cyclosporin A, Surfonic LF®, and Caraway oil.

Another embodiment comprises cyclosporin A, T-Det A826®, and Caraway oil.

Another embodiment comprises cyclosporin A, T-Det LF-416®, and Caraway oil.

Another embodiment comprises cyclosporin A, Tergitol Min-Foam 1X®, and Caraway oil.

Another embodiment comprises cyclosporin A, Tergitol Min-Foam 2X®, and Caraway oil.

Another embodiment comprises cyclosporin A, Triton CF-21®, and Caraway oil.

Another embodiment comprises cyclosporin A, Triton CF-76®, and Caraway oil.

Another embodiment comprises cyclosporin A, Triton XL-80N®, and Caraway oil.

Another embodiment comprises cyclosporin A, Witconol NS-98®, and Caraway oil.

Another embodiment comprises cyclosporin A, Witconol NS-108LQ®, and Caraway oil.

Another embodiment comprises cyclosporin A, Witconol NS-145®, and Caraway oil.

Another embodiment comprises cyclosporin A, Witconol NS-179®, and Caraway oil.

Another embodiment comprises cyclosporin A, a Protein-based Surfactant, and Caraway oil.

Another embodiment comprises cyclosporin A, AminoFoam W®, and Caraway oil.

Another embodiment comprises cyclosporin A, Amiter LGOD-2®, and Caraway oil.

Another embodiment comprises cyclosporin A, Amiter LGS-2®, and Caraway oil.

Another embodiment comprises cyclosporin A, Amiter LGS-5®, and Caraway oil.

Another embodiment comprises cyclosporin A, Lamepon S®, and Caraway oil.

Another embodiment comprises cyclosporin A, Lamepon S®, and Caraway oil.

Another embodiment comprises cyclosporin A, Maypon 4C®, and Caraway oil.

Another embodiment comprises cyclosporin A, May-Tein C®, and Caraway oil.

Another embodiment comprises cyclosporin A, May-Tein CT®, and Caraway oil.

Another embodiment comprises cyclosporin A, May-Tein KTS®, and Caraway oil.

Another embodiment comprises cyclosporin A, May-Tein SY®, and Caraway oil.

Another embodiment comprises cyclosporin A, Plantapon S®, and Caraway oil.

Another embodiment comprises cyclosporin A, Proteol APL®, and Caraway oil.

Another embodiment comprises cyclosporin A, Proteol OAT®, and Caraway oil.

Another embodiment comprises cyclosporin A, Pyroter CPI-40®, and Caraway oil.

Another embodiment comprises cyclosporin A, Pyroter GPI-25®, and Caraway oil.

Another embodiment comprises cyclosporin A, Supro-Tein S®, and Caraway oil.

Another embodiment comprises cyclosporin A, Supro-Tein V®, and Caraway oil.

Another embodiment comprises cyclosporin A,® and Caraway oil.

Another embodiment comprises cyclosporin A, a Sarcosine Derivative®, and Caraway oil.

Another embodiment comprises cyclosporin A, Crodasinic LS-30®, and Caraway oil.

Another embodiment comprises cyclosporin A, Vanseal CS®, and Caraway oil.

Another embodiment comprises cyclosporin A, Vanseal LS®, and Caraway oil.

Another embodiment comprises cyclosporin A, Vanseal MS®, and Caraway oil.

Another embodiment comprises cyclosporin A, Vanseal NACS-30®, and Caraway oil.

Another embodiment comprises cyclosporin A, Vanseal NALS-95®, and Caraway oil.

Another embodiment comprises cyclosporin A, Vanseal OS®, and Caraway oil.

Another embodiment comprises cyclosporin A,® and Caraway oil.

Another embodiment comprises cyclosporin A, a Silicone-based Surtactant®, and Caraway oil.

Another embodiment comprises cyclosporin A, Abil-B-9950®, and Caraway oil.

Another embodiment comprises cyclosporin A, Abil Care 85®, and Caraway oil.

Another embodiment comprises cyclosporin A, Abil EM 90®, and Caraway oil.

Another embodiment comprises cyclosporin A, Abil EM 97®, and Caraway oil.

Another embodiment comprises cyclosporin A, Abil WE-09®, and Caraway oil.

Another embodiment comprises cyclosporin A, Dow Corning 1248 Fluid®, and Caraway oil.

Another embodiment comprises cyclosporin A, Dow Corning 3225C®, and Caraway oil.

Another embodiment comprises cyclosporin A, Dow Corning 5200®, and Caraway oil.

Another embodiment comprises cyclosporin A, Dow Corning Q4-3667®, and Caraway oil.

Another embodiment comprises cyclosporin A, Monasil PCA®, and Caraway oil.

Another embodiment comprises cyclosporin A, Monasil PDM®, and Caraway oil.

Another embodiment comprises cyclosporin A, Monasil PLN®, and Caraway oil.

Another embodiment comprises cyclosporin A, Polyderm PPI-SI-WS®, and Caraway oil.

Another embodiment comprises cyclosporin A, Troysol 380W®, and Caraway oil.

Another embodiment comprises cyclosporin A, Troysol S366®, and Caraway oil.

Another embodiment comprises cyclosporin A, a Sorbitan Derivative, and Caraway oil.

Another embodiment comprises cyclosporin A, Alkamuls SML®, and Caraway oil.

Another embodiment comprises cyclosporin A, Alkamuls SMO®, and Caraway oil.

Another embodiment comprises cyclosporin A, Alkamuls STO®, and Caraway oil.

Another embodiment comprises cyclosporin A, Arlacel 20®, and Caraway oil.

Another embodiment comprises cyclosporin A, Arlacel 40®, and Caraway oil.

Another embodiment comprises cyclosporin A, Arlacel 60®, and Caraway oil.

Another embodiment comprises cyclosporin A, Arlacel 80®, and Caraway oil.

Another embodiment comprises cyclosporin A, Arlacel C®, and Caraway oil.

Another embodiment comprises cyclosporin A, Armul 21®, and Caraway oil.

Another embodiment comprises cyclosporin A, Atlox 80®, and Caraway oil.

Another embodiment comprises cyclosporin A, Atlox 847®, and Caraway oil.

Another embodiment comprises cyclosporin A, Atlox 1045A®, and Caraway oil.

Another embodiment comprises cyclosporin A, Canarcel 20®, and Caraway oil.

Another embodiment comprises cyclosporin A, Canarcel 60®, and Caraway oil.

Another embodiment comprises cyclosporin A, Canarcel 80®, and Caraway oil.

Another embodiment comprises cyclosporin A, Canarcel TW 20®, and Caraway oil.

Another embodiment comprises cyclosporin A, Canarcel TW 60®, and Caraway oil.

Another embodiment comprises cyclosporin A, Canarcel TW 80®, and Caraway oil.

Another embodiment comprises cyclosporin A, Coladet BSB-P®, and Caraway oil.

Another embodiment comprises cyclosporin A, Customulse O-20®, and Caraway oil.

Another embodiment comprises cyclosporin A, Dehymuls E®, and Caraway oil.

Another embodiment comprises cyclosporin A, DeSotan SMO®, and Caraway oil.

Another embodiment comprises cyclosporin A, DeSotan SMO-20®, and Caraway oil.

Another embodiment comprises cyclosporin A, DeSotan SMT®, and Caraway oil.

Another embodiment comprises cyclosporin A, DeSotan SMT-20®, and Caraway oil.

Another embodiment comprises cyclosporin A, Durfax 60®, and Caraway oil.

Another embodiment comprises cyclosporin A, Durfax 65®, and Caraway oil.

Another embodiment comprises cyclosporin A, Durdax 80®, and Caraway oil.

Another embodiment comprises cyclosporin A, Durtan 60®, and Caraway oil.

Another embodiment comprises cyclosporin A, Durtan 65®, and Caraway oil.

Another embodiment comprises cyclosporin A, Liposorb L®, and Caraway oil.

Another embodiment comprises cyclosporin A, Liposorb L-10®, and Caraway oil.

Another embodiment comprises cyclosporin A, Liposorb L-20®, and Caraway oil.

Another embodiment comprises cyclosporin A, Liposorb O®, and Caraway oil.

Another embodiment comprises cyclosporin A, Liposorb O-20®, and Caraway oil.

Another embodiment comprises cyclosporin A, Liposorb P®, and Caraway oil.

Another embodiment comprises cyclosporin A, Liposorb P-20®, and Caraway oil.

Another embodiment comprises cyclosporin A, Liposorb S®, and Caraway oil.

Another embodiment comprises cyclosporin A, Liposorb S-20®, and Caraway oil.

Another embodiment comprises cyclosporin A, Liposorb SQO®, and Caraway oil.

Another embodiment comprises cyclosporin A, Liposorb TO®, and Caraway oil.

Another embodiment comprises cyclosporin A, Liposorb TS®, and Caraway oil.

Another embodiment comprises cyclosporin A, Liposorb TS-20®, and Caraway oil.

Another embodiment comprises cyclosporin A, Lumisorb PS®, and Caraway oil.

Another embodiment comprises cyclosporin A, Lumisorb SMO (T)®, and Caraway oil.

Another embodiment comprises cyclosporin A, Lumisorb SMS K®, and Caraway oil.

Another embodiment comprises cyclosporin A, Lumisorb SSO®, and Caraway oil.

Another embodiment comprises cyclosporin A, Lumisorb STS K®, and Caraway oil.

Another embodiment comprises cyclosporin A, Lumisorb STT®, and Caraway oil.

Another embodiment comprises cyclosporin A, Magrabar SMO®, and Caraway oil.

Another embodiment comprises cyclosporin A, Magrabar SMO-VEG®, and Caraway oil.

Another embodiment comprises cyclosporin A, Magrabar SMT®, and Caraway oil.

Another embodiment comprises cyclosporin A, Magrabar STO®, and Caraway oil.

Another embodiment comprises cyclosporin A, Miracare BC-27®, and Caraway oil.

Another embodiment comprises cyclosporin A, Ritabate 20®, and Caraway oil.

Another embodiment comprises cyclosporin A, Ritabate 40®, and Caraway oil.

Another embodiment comprises cyclosporin A, Ritabate 60®, and Caraway oil.

Another embodiment comprises cyclosporin A, Ritabate 80®, and Caraway oil.

Another embodiment comprises cyclosporin A, T-Maz®, and Caraway oil.

Another embodiment comprises cyclosporin A, Tego SML®, and Caraway oil.

Another embodiment comprises cyclosporin A, Tego SML 20®, and Caraway oil.

Another embodiment comprises cyclosporin A, Tego SMO 80 V®, and Caraway oil.

Another embodiment comprises cyclosporin A, Tego SMO V®, and Caraway oil.

Another embodiment comprises cyclosporin A, Tego SMS®, and Caraway oil.

Another embodiment comprises cyclosporin A, Tego STO V®, and Caraway oil.

Another embodiment comprises cyclosporin A, Tween 21®, and Caraway oil.

Another embodiment comprises cyclosporin A, Tween 40®, and Caraway oil.

Another embodiment comprises cyclosporin A, Tween 60®, and Caraway oil.

Another embodiment comprises cyclosporin A, Tween 60 K®, and Caraway oil.

Another embodiment comprises cyclosporin A, Tween 61®, and Caraway oil.

Another embodiment comprises cyclosporin A, Tween 65®, and Caraway oil.

Another embodiment comprises cyclosporin A, Tween 80®, and Caraway oil.

Another embodiment comprises cyclosporin A, Tween 80 K®, and Caraway oil.

Another embodiment comprises cyclosporin A, Tween 81®, and Caraway oil.

Another embodiment comprises cyclosporin A, Tween 85®, and Caraway oil.

Another embodiment comprises cyclosporin A, a Sucrose or Glucose Ester, or Derivative thereof, and Caraway oil.

Another embodiment comprises cyclosporin A, DeSulf GOS-P-60WCG®, and Caraway oil.

Another embodiment comprises cyclosporin A, Glucam E-20 Distearate®, and Caraway oil.

Another embodiment comprises cyclosporin A, Glucamate DOE-120®, and Caraway oil.

Another embodiment comprises cyclosporin A, Glucamate SSE-20®, and Caraway oil.

Another embodiment comprises cyclosporin A, Glucate DO®, and Caraway oil.

Another embodiment comprises cyclosporin A, Glucate SS®, and Caraway oil.

Another embodiment comprises cyclosporin A, Glucopon 425 UP®, and Caraway oil.

Another embodiment comprises cyclosporin A, Isolan IS®, and Caraway oil.

Another embodiment comprises cyclosporin A, Mazon 40®, and Caraway oil.

Another embodiment comprises cyclosporin A, Montanov 82®, and Caraway oil.

Another embodiment comprises cyclosporin A, Montanov 202®, and Caraway oil.

Another embodiment comprises cyclosporin A, Montanov S®, and Caraway oil.

Another embodiment comprises cyclosporin A, Rheozan®, and Caraway oil.

Another embodiment comprises cyclosporin A, Simulsol AS 48®, and Caraway oil.

Another embodiment comprises cyclosporin A, Simulsol SL 4®, and Caraway oil.

Another embodiment comprises cyclosporin A, Simulsol SL 10®, and Caraway oil.

Another embodiment comprises cyclosporin A, Simulsol SL 11W®, and Caraway oil.

Another embodiment comprises cyclosporin A, Simulsol SL 55®, and Caraway oil.

Another embodiment comprises cyclosporin A, Suga Nate 100 and 160®, and Caraway oil.

Another embodiment comprises cyclosporin A, Tego Care 450®, and Caraway oil.

Another embodiment comprises cyclosporin A, Tego Care CG 90®, and Caraway oil.

Another embodiment comprises cyclosporin A, Tego Care PS®, and Caraway oil.

Another embodiment comprises cyclosporin A, Tegosoft PSE 141 G®, and Caraway oil.

Another embodiment comprises cyclosporin A, Tegotens G 826®, and Caraway oil.

Another embodiment comprises cyclosporin A, Triton BG-10 (70%)®, and Caraway oil.

Another embodiment comprises cyclosporin A, Triton CG-110 (60%)®, and Caraway oil.

Another embodiment comprises cyclosporin A, Wickenol 545®, and Caraway oil.

Another embodiment comprises cyclosporin A, an alcohol, and Rosemary oil.

Another embodiment comprises cyclosporin A, Diglycerol®, and Rosemary oil.

Another embodiment comprises cyclosporin A, Hetoxide GT-80®, and Rosemary oil.

Another embodiment comprises cyclosporin A, Lexemul BEO®, and Rosemary oil.

Another embodiment comprises cyclosporin A, Polyglycerol-3®, and Rosemary oil.

Another embodiment comprises cyclosporin A, Redicote E Series®, and Rosemary oil.

Another embodiment comprises cyclosporin A, Simulsol OX 1005L®, and Rosemary oil.

Another embodiment comprises cyclosporin A, Stanfax 567®, and Rosemary oil.

Another embodiment comprises cyclosporin A, TA-1618®, and Rosemary oil.

Another embodiment comprises cyclosporin A, Witconol H-31A®, and Rosemary oil.

Another embodiment comprises cyclosporin A, an amine oxide, and Rosemary oil.

Another embodiment comprises cyclosporin A, AO-405®, and Rosemary oil.

Another embodiment comprises cyclosporin A, AO-455®, and Rosemary oil.

Another embodiment comprises cyclosporin A, AO 728 Special®, and Rosemary oil.

Another embodiment comprises cyclosporin A, Barlox 12®, and Rosemary oil.

Another embodiment comprises cyclosporin A, Barlox 14®, and Rosemary oil.

Another embodiment comprises cyclosporin A, Burcoxide Lo®, and Rosemary oil.

Another embodiment comprises cyclosporin A, Caloxamine LO®, and Rosemary oil.

Another embodiment comprises cyclosporin A, Chemoxide CAW®, and Rosemary oil.

Another embodiment comprises cyclosporin A, Chemoxide LM-30®, and Rosemary oil.

Another embodiment comprises cyclosporin A, Chemoxide LO®, and Rosemary oil.

Another embodiment comprises cyclosporin A, Chemoxide MO®, and Rosemary oil.

Another embodiment comprises cyclosporin A, Colalux CAO-35®, and Rosemary oil.

Another embodiment comprises cyclosporin A, Colalux LO®, and Rosemary oil.

Another embodiment comprises cyclosporin A, DeMox CAPO®, and Rosemary oil.

Another embodiment comprises cyclosporin A, DeMox CSG-30®, and Rosemary oil.

Another embodiment comprises cyclosporin A, DeMox LAO®, and Rosemary oil.

Another embodiment comprises cyclosporin A, Emcol L®, and Rosemary oil.

Another embodiment comprises cyclosporin A, Empigen OB®, and Rosemary oil.

Another embodiment comprises cyclosporin A, Empigen OS/A®, and Rosemary oil.

Another embodiment comprises cyclosporin A, Foamox CDO®, and Rosemary oil.

Another embodiment comprises cyclosporin A, Foamox DMM®, and Rosemary oil.

Another embodiment comprises cyclosporin A, Foamox DMS®, and Rosemary oil.

Another embodiment comprises cyclosporin A, Genaminox KC®, and Rosemary oil.

Another embodiment comprises cyclosporin A, Genaminox LA®, and Rosemary oil.

Another embodiment comprises cyclosporin A, Hartofoam SAO®, and Rosemary oil.

Another embodiment comprises cyclosporin A, Hartox DMCD®, and Rosemary oil.

Another embodiment comprises cyclosporin A, Lipowax DAT®, and Rosemary oil.

Another embodiment comprises cyclosporin A, Lipowax PB Pastilles®, and Rosemary oil.

Another embodiment comprises cyclosporin A, Mackamine C8®, and Rosemary oil.

Another embodiment comprises cyclosporin A, Mackamine C10®, and Rosemary oil.

Another embodiment comprises cyclosporin A, Mackamine C14®, and Rosemary oil.

Another embodiment comprises cyclosporin A, Mackamine CAO®, and Rosemary oil.

Another embodiment comprises cyclosporin A, Mackamine CO®, and Rosemary oil.

Another embodiment comprises cyclosporin A, Mackamine LO®, and Rosemary oil.

Another embodiment comprises cyclosporin A, Mackamine O2®, and Rosemary oil.

Another embodiment comprises cyclosporin A, Mackamine SAO®, and Rosemary oil.

Another embodiment comprises cyclosporin A, Mackamine SO®, and Rosemary oil.

Another embodiment comprises cyclosporin A, Mazox KCAO®, and Rosemary oil.

Another embodiment comprises cyclosporin A, Monalac MO®, and Rosemary oil.

Another embodiment comprises cyclosporin A, Norfox LDA®, and Rosemary oil.

Another embodiment comprises cyclosporin A, Rhodamox LO®, and Rosemary oil.

Another embodiment comprises cyclosporin A, Schercamox C-AA®, and Rosemary oil.

Another embodiment comprises cyclosporin A, Schercamox DMA®, and Rosemary oil.

Another embodiment comprises cyclosporin A, Schercamox DML®, and Rosemary oil.

Another embodiment comprises cyclosporin A, Schercamox DMM®, and Rosemary oil.

Another embodiment comprises cyclosporin A, Schercamox DMS®, and Rosemary oil.

Another embodiment comprises cyclosporin A, Tegotens DO®, and Rosemary oil.

Another embodiment comprises cyclosporin A, Tomah AO-14-2®, and Rosemary oil.

Another embodiment comprises cyclosporin A, Triaminox CDO®, and Rosemary oil.

Another embodiment comprises cyclosporin A, a block polymer, and Rosemary oil.

Another embodiment comprises cyclosporin A, AL 2070®, and Rosemary oil.

Another embodiment comprises cyclosporin A, Antarox 17-R-2®, and Rosemary oil.

Another embodiment comprises cyclosporin A, Antarox 25-R-2®, and Rosemary oil.

Another embodiment comprises cyclosporin A, Antarox 31 -R-1®, and Rosemary oil.

Another embodiment comprises cyclosporin A, Antarox P-84®, and Rosemary oil.

Another embodiment comprises cyclosporin A, Antarox P-104/H®, and Rosemary oil.

Another embodiment comprises cyclosporin A, Arnox BP-Series®, and Rosemary oil.

Another embodiment comprises cyclosporin A, Chemonic 435®, and Rosemary oil.

Another embodiment comprises cyclosporin A, Chemonic D-25®, and Rosemary oil.

Another embodiment comprises cyclosporin A, Chemonic PL Series®, and Rosemary oil.

Another embodiment comprises cyclosporin A, Ethox L-121®, and Rosemary oil.

Another embodiment comprises cyclosporin A, Ethox L-122®, and Rosemary oil.

Another embodiment comprises cyclosporin A, Genapol PF-10®, and Rosemary oil.

Another embodiment comprises cyclosporin A, Genapol PF-20®, and Rosemary oil.

Another embodiment comprises cyclosporin A, Genapol PF-40A®, and Rosemary oil.

Another embodiment comprises cyclosporin A, Norfox 2-LF®, and Rosemary oil.

Another embodiment comprises cyclosporin A, Simulsol NW 342®, and Rosemary oil.

Another embodiment comprises cyclosporin A, T-Det BP-1®, and Rosemary oil.

Another embodiment comprises cyclosporin A, T-Det XD®, and Rosemary oil.

Another embodiment comprises cyclosporin A, T-Det XH®, and Rosemary oil.

Another embodiment comprises cyclosporin A, Triton CF-32®, and Rosemary oil.

Another embodiment comprises cyclosporin A, Witconol 171®, and Rosemary oil.

Another embodiment comprises cyclosporin A, Witconol 324®, and Rosemary oil.

Another embodiment comprises cyclosporin A, Witconol 324D®, and Rosemary oil.

Another embodiment comprises cyclosporin A, Witconol PD-2000®, and Rosemary oil.

Another embodiment comprises cyclosporin A, a Carboxylated Alcohol or Alkylphenol Ethoxylate, and Rosemary oil.

Another embodiment comprises cyclosporin A, Emcol CN-6®, and Rosemary oil.

Another embodiment comprises cyclosporin A, Ethcarb®, and Rosemary oil.

Another embodiment comprises cyclosporin A, Gemtex WNT-Conc®, and Rosemary oil.

Another embodiment comprises cyclosporin A, Incrodet TD7-C®, and Rosemary oil.

Another embodiment comprises cyclosporin A, Marlinat CM 105/80®, and Rosemary oil.

Another embodiment comprises cyclosporin A, Marlowet 1072®, and Rosemary oil.

Another embodiment comprises cyclosporin A, Marlowet 4530®, and Rosemary oil.

Another embodiment comprises cyclosporin A, Marlowet 4530 LF®, and Rosemary oil.

Another embodiment comprises cyclosporin A, Marlowet 4534®, and Rosemary oil.

Another embodiment comprises cyclosporin A, Marlowet 4538®, and Rosemary oil.

Another embodiment comprises cyclosporin A, Marlowet 4539®, and Rosemary oil.

Another embodiment comprises cyclosporin A, Marlowet 4539 LF®, and Rosemary oil.

Another embodiment comprises cyclosporin A, Marlowet 4541®, and Rosemary oil.

Another embodiment comprises cyclosporin A, Miranate LEC-80®, and Rosemary oil.

Another embodiment comprises cyclosporin A, Sandopan B®, and Rosemary oil.

Another embodiment comprises cyclosporin A, Sandopan B Modified®, and Rosemary oil.

Another embodiment comprises cyclosporin A, Sandopan LS-24 Gel®, and Rosemary oil.

Another embodiment comprises cyclosporin A, Surfine T-A®, and Rosemary oil.

Another embodiment comprises cyclosporin A, a Carboxylic Acid/Fatty Acid, and Rosemary oil.

Another embodiment comprises cyclosporin A, Colaterge RAM®, and Rosemary oil.

Another embodiment comprises cyclosporin A, Colatrope INC®, and Rosemary oil.

Another embodiment comprises cyclosporin A, Crodacid B®, and Rosemary oil.

Another embodiment comprises cyclosporin A, DeTrope CA-100®, and Rosemary oil.

Another embodiment comprises cyclosporin A, Latol MTO®, and Rosemary oil.

Another embodiment comprises cyclosporin A, Lumulse CC-33 K®, and Rosemary oil.

Another embodiment comprises cyclosporin A, Mulls 2218®, and Rosemary oil.

Another embodiment comprises cyclosporin A, OL-600®, and Rosemary oil.

Another embodiment comprises cyclosporin A, OL-800®, and Rosemary oil.

Another embodiment comprises cyclosporin A, R-910®, and Rosemary oil.

Another embodiment comprises cyclosporin A, S-210®, and Rosemary oil.

Another embodiment comprises cyclosporin A, Sandopan DTC Acid®, and Rosemary oil.

Another embodiment comprises cyclosporin A, Sandopan LS 24 N®, and Rosemary oil.

Another embodiment comprises cyclosporin A, Sandopan MA-18®, and Rosemary oil.

Another embodiment comprises cyclosporin A, an Ethoxylated Alcohol, and Rosemary oil.

Another embodiment comprises cyclosporin A, Adsee 799®, and Rosemary oil.

Another embodiment comprises cyclosporin A, Adsee 799®, and Rosemary oil.

Another embodiment comprises cyclosporin A, Alfonic 610-3.5®, and Rosemary oil.

Another embodiment comprises cyclosporin A, Alfonic 810-2®, and Rosemary oil.

Another embodiment comprises cyclosporin A, Alfonic 810-6®, and Rosemary oil.

Another embodiment comprises cyclosporin A, Alfonic 1012-3®, and Rosemary oil.

Another embodiment comprises cyclosporin A, Alfonic 1012-5®, and Rosemary oil.

Another embodiment comprises cyclosporin A, Alfonic 1216CO-1.5®, and Rosemary oil.

Another embodiment comprises cyclosporin A, Alfonic 1216CO-7®, and Rosemary oil.

Another embodiment comprises cyclosporin A, Alfonic 1412-3®, and Rosemary oil.

Another embodiment comprises cyclosporin A, Alfonic 1412-7®, and Rosemary oil.

Another embodiment comprises cyclosporin A, Arlasolve 200®, and Rosemary oil.

Another embodiment comprises cyclosporin A, Arlasolve 200 Liquid®, and Rosemary oil.

Another embodiment comprises cyclosporin A, Armix 180-C®, and Rosemary oil.

Another embodiment comprises cyclosporin A, Armix 183®, and Rosemary oil.

Another embodiment comprises cyclosporin A, Armul 2404®, and Rosemary oil.

Another embodiment comprises cyclosporin A, Armul 2404®, and Rosemary oil.

Another embodiment comprises cyclosporin A, Atlas EMJ-C®, and Rosemary oil.

Another embodiment comprises cyclosporin A, Atlas G-2109®, and Rosemary oil.

Another embodiment comprises cyclosporin A, Atlas G-3886®, and Rosemary oil.

Another embodiment comprises cyclosporin A, Atlas G-3890®, and Rosemary oil.

Another embodiment comprises cyclosporin A, Bio Soft E-200®, and Rosemary oil.

Another embodiment comprises cyclosporin A, Bio Soft E-300®, and Rosemary oil.

Another embodiment comprises cyclosporin A, Bio Soft E-400®, and Rosemary oil.

Another embodiment comprises cyclosporin A, Bio Soft EN 600®, and Rosemary oil.

Another embodiment comprises cyclosporin A, Bio Soft TD-400®, and Rosemary oil.

Another embodiment comprises cyclosporin A, Bio Soft TD-630®, and Rosemary oil.

Another embodiment comprises cyclosporin A, Brij 30®, and Rosemary oil.

Another embodiment comprises cyclosporin A, Brij 52®, and Rosemary oil.

Another embodiment comprises cyclosporin A, Brij 56®, and Rosemary oil.

Another embodiment comprises cyclosporin A, Brij 58®, and Rosemary oil.

Another embodiment comprises cyclosporin A, Brij 72®, and Rosemary oil.

Another embodiment comprises cyclosporin A, Brij 76®, and Rosemary oil.

Another embodiment comprises cyclosporin A, Brij 78®, and Rosemary oil.

Another embodiment comprises cyclosporin A, Brij 93®, and Rosemary oil.

Another embodiment comprises cyclosporin A, Brij 97®, and Rosemary oil.

Another embodiment comprises cyclosporin A, Brij 98®, and Rosemary oil.

Another embodiment comprises cyclosporin A, Brij 700®, and Rosemary oil.

Another embodiment comprises cyclosporin A, Brij 700 S®, and Rosemary oil.

Another embodiment comprises cyclosporin A, Brij 721®, and Rosemary oil.

Another embodiment comprises cyclosporin A, Brij 721 S®, and Rosemary oil.

Another embodiment comprises cyclosporin A, Burcoterge CDG®, and Rosemary oil.

Another embodiment comprises cyclosporin A, Canasol AT 600®, and Rosemary oil.

Another embodiment comprises cyclosporin A, Canasol AT 800®, and Rosemary oil.

Another embodiment comprises cyclosporin A, Canasol AT 1200®, and Rosemary oil.

Another embodiment comprises cyclosporin A, Canasbl BJ 35®, and Rosemary oil.

Another embodiment comprises cyclosporin A, Canasol BJ 36®, and Rosemary oil.

Another embodiment comprises cyclosporin A, Canasol BJ 52®, and Rosemary oil.

Another embodiment comprises cyclosporin A, Canasol BJ 58®, and Rosemary oil.

Another embodiment comprises cyclosporin A, Canasol BJ 72®, and Rosemary oil.

Another embodiment comprises cyclosporin A, Canasol BJ 78®, and Rosemary oil.

Another embodiment comprises cyclosporin A, Canasol BJ 98®, and Rosemary oil.

Another embodiment comprises cyclosporin A, Canasol BJ 307®, and Rosemary oil.

Another embodiment comprises cyclosporin A, Cerfak 1400®, and Rosemary oil.

Another embodiment comprises cyclosporin A, Cetomacrogol 1000 BP®, and Rosemary oil.

Another embodiment comprises cyclosporin A, Chemonic C-2®, and Rosemary oil.

Another embodiment comprises cyclosporin A, Chemonic C-10®, and Rosemary oil.

Another embodiment comprises cyclosporin A, Chemonic C-20®, and Rosemary oil.

Another embodiment comprises cyclosporin A, Chemonic CT-12®, and Rosemary oil.

Another embodiment comprises cyclosporin A, Chemonic CT-20®, and Rosemary oil.

Another embodiment comprises cyclosporin A, Chemonic CT-30®, and Rosemary oil.

Another embodiment comprises cyclosporin A, Chemonic CT-55®, and Rosemary oil.

Another embodiment comprises cyclosporin A, Chemonic G-7®, and Rosemary oil.

Another embodiment comprises cyclosporin A, Chemonic G-26®, and Rosemary oil.

Another embodiment comprises cyclosporin A, Chemonic L-4®, and Rosemary oil.

Another embodiment comprises cyclosporin A, Chemonic L-7®, and Rosemary oil.

Another embodiment comprises cyclosporin A, Chemonic L-12®, and Rosemary oil.

Another embodiment comprises cyclosporin A, Chemonic L-23®, and Rosemary oil.

Another embodiment comprises cyclosporin A, Chemonic O-2®, and Rosemary oil.

Another embodiment comprises cyclosporin A, Chemonic O-5®, and Rosemary oil.

Another embodiment comprises cyclosporin A, Chemonic O-10®, and Rosemary oil.

Another embodiment comprises cyclosporin A, Chemonic O-20®, and Rosemary oil.

Another embodiment comprises cyclosporin A, Chemonic S-2®, and Rosemary oil.

Another embodiment comprises cyclosporin A, Chemonic S-10®, and Rosemary oil.

Another embodiment comprises cyclosporin A, Chemonic S-20®, and Rosemary oil.

Another embodiment comprises cyclosporin A, Colamulse FE®, and Rosemary oil.

Another embodiment comprises cyclosporin A, Cremophor A 20®, and Rosemary oil.

Another embodiment comprises cyclosporin A, Cremophor SA 2®, and Rosemary oil.

Another embodiment comprises cyclosporin A, Dehydol 100®, and Rosemary oil.

Another embodiment comprises cyclosporin A, Dehydol O-4®, and Rosemary oil.

Another embodiment comprises cyclosporin A, Delonic C-18®, and Rosemary oil.

Another embodiment comprises cyclosporin A, DeSonic 6T®, and Rosemary oil.

Another embodiment comprises cyclosporin A, DeSonic 9D®, and Rosemary oil.

Another embodiment comprises cyclosporin A, DeSonic 9T®, and Rosemary oil.

Another embodiment comprises cyclosporin A, DeSonic 12D®, and Rosemary oil.

Another embodiment comprises cyclosporin A, DeSonic 12T®, and Rosemary oil.

Another embodiment comprises cyclosporin A, DeSonic 15T®, and Rosemary oil.

Another embodiment comprises cyclosporin A, DeSonic TDA-9®, and Rosemary oil.

Another embodiment comprises cyclosporin A, DeThox GLG-7®, and Rosemary oil.

Another embodiment comprises cyclosporin A, DeThox GLG-26®, and Rosemary oil.

Another embodiment comprises cyclosporin A, DeThox LA-4®, and Rosemary oil.

Another embodiment comprises cyclosporin A, DeThox LA-23®, and Rosemary oil.

Another embodiment comprises cyclosporin A, DeThox SA-80®, and Rosemary oil.

Another embodiment comprises cyclosporin A, Disponil O5®, and Rosemary oil.

Another embodiment comprises cyclosporin A, Eccoterge EO-41B®, and Rosemary oil.

Another embodiment comprises. cyclosporin A, Empilan KA2.5/90®, and Rosemary oil.

Another embodiment comprises cyclosporin A, Empilan KA5/90®, and Rosemary oil.

Another embodiment comprises cyclosporin A, Empilan KM-20®, and Rosemary oil.

Another embodiment comprises cyclosporin A, Empilan KM-50®, and Rosemary oil.

Another embodiment comprises cyclosporin A, Empilan L-23®, and Rosemary oil.

Another embodiment comprises cyclosporin A, Ethylan 25-3®, and Rosemary oil.

Another embodiment comprises cyclosporin A, Ethylan 1204®, and Rosemary oil.

Another embodiment comprises cyclosporin A, Ethylan DA-4®, and Rosemary oil.

Another embodiment comprises cyclosporin A, Ethylan LA-230®, and Rosemary oil.

Another embodiment comprises cyclosporin A, Ethylan SN®, and Rosemary oil.

Another embodiment comprises cyclosporin A, Ethylan TD-60®, and Rosemary oil.

Another embodiment comprises cyclosporin A, Ethylan TD-100®, and Rosemary oil.

Another embodiment comprises cyclosporin A, Ethylan TD-1407®, and Rosemary oil.

Another embodiment comprises cyclosporin A, Eumulgin B1®, and Rosemary oil.

Another embodiment comprises cyclosporin A, Eumulgin B2®, and Rosemary oil.

Another embodiment comprises cyclosporin A, Eumulgin B3®, and Rosemary oil.

Another embodiment comprises cyclosporin A, Eumulgin O-10®, and Rosemary oil.

Another embodiment comprises cyclosporin A, Flo Mo 80/20®, and Rosemary oil.

Another embodiment comprises cyclosporin A, Flo Mo Low Foam®, and Rosemary oil.

Another embodiment comprises cyclosporin A, Forlan C-24®, and Rosemary oil.

Another embodiment comprises cyclosporin A, Genapol 1454®, and Rosemary oil.

Another embodiment comprises cyclosporin A, Genapol BA-020®, and Rosemary oil.

Another embodiment comprises cyclosporin A, Genapol BA-040®, and Rosemary oil.

Another embodiment comprises cyclosporin A, Genapol C-100®, and Rosemary oil.

Another embodiment comprises cyclosporin A, Genapol DA 060®, and Rosemary oil.

Another embodiment comprises cyclosporin A, Genapol HS 020®, and Rosemary oil.

Another embodiment comprises cyclosporin A, Genapol HS 200®, and Rosemary oil.

Another embodiment comprises cyclosporin A, Genapol ID-040®, and Rosemary oil.

Another embodiment comprises cyclosporin A, Genapol ID-060®, and Rosemary oil.

Another embodiment comprises cyclosporin A, Genapol ID-090®, and Rosemary oil.

Another embodiment comprises cyclosporin A, Genapol LA 010®, and Rosemary oil.

Another embodiment comprises cyclosporin A, Genapol LA 020®, and Rosemary oil.

Another embodiment comprises cyclosporin A, Genapol LA 030®, and Rosemary oil.

Another embodiment comprises cyclosporin A, Genapol LA 040®, and Rosemary oil.

Another embodiment comprises cyclosporin A, Genapol LA 050®, and Rosemary oil.

Another embodiment comprises cyclosporin A, Genapol LA 060®, and Rosemary oil.

Another embodiment comprises cyclosporin A, Genapol LA 070®, and Rosemary oil.

Another embodiment comprises cyclosporin A, Genapol LA 070S®, and Rosemary oil.

Another embodiment comprises cyclosporin A, Genapol LA 230®, and Rosemary oil.

Another embodiment comprises cyclosporin A, Genapol O 020®, and Rosemary oil.

Another embodiment comprises cyclosporin A, Genapol O050®, and Rosemary oil.

Another embodiment comprises cyclosporin A, Genapol O100®, and Rosemary oil.

Another embodiment comprises cyclosporin A, Genapol O200®, and Rosemary oil.

Another embodiment comprises cyclosporin A, Genapol SA 030®, and Rosemary oil.

Another embodiment comprises cyclosporin A, Genapol SA 120®, and Rosemary oil.

Another embodiment comprises cyclosporin A, Genapol T-020®, and Rosemary oil.

Another embodiment comprises cyclosporin A, Genapol UD-030®, and Rosemary oil.

Another embodiment comprises cyclosporin A, Genapol UD-050®, and Rosemary oil.

Another embodiment comprises cyclosporin A, Genapol UD-070®, and Rosemary oil.

Another embodiment comprises cyclosporin A, Genapol UD-079®, and Rosemary oil.

Another embodiment comprises cyclosporin A, Genapol UD-080®, and Rosemary oil.

Another embodiment comprises cyclosporin A, Genapol UD-110®, and Rosemary oil.

Another embodiment comprises cyclosporin A, Genapol X 030®, and Rosemary oil.

Another embodiment comprises cyclosporin A, Genapol X 050®, and Rosemary oil.

Another embodiment comprises cyclosporin A, Genapol X 060®, and Rosemary oil.

Another embodiment comprises cyclosporin A, Genapol X 070®, and Rosemary oil.

Another embodiment comprises cyclosporin A, Genapol X 080®, and Rosemary oil.

Another embodiment comprises cyclosporin A, Genapol X 100®, and Rosemary oil.

Another embodiment comprises cyclosporin A, Genapol X 159®, and Rosemary oil.

Another embodiment comprises cyclosporin A, Generol 122 E5®, and Rosemary oil.

Another embodiment comprises cyclosporin A, Generol 122 E25®, and Rosemary oil.

Another embodiment comprises cyclosporin A, Hostacerin T-3®, and Rosemary oil.

Another embodiment comprises cyclosporin A, Iconol LF 110®, and Rosemary oil.

Another embodiment comprises cyclosporin A, Incropol CS-20®, and Rosemary oil.

Another embodiment comprises cyclosporin A, Lexemul CS-20®, and Rosemary oil.

Another embodiment comprises cyclosporin A, Liponic EG-1®, and Rosemary oil.

Another embodiment comprises cyclosporin A, Lipowax D®, and Rosemary oil.

Another embodiment comprises cyclosporin A, Lipowax G®, and Rosemary oil.

Another embodiment comprises cyclosporin A, Lipowax NI®, and Rosemary oil.

Another embodiment comprises cyclosporin A, Lipowax P®, and Rosemary oil.

Another embodiment comprises cyclosporin A, Lipowax P-31®, and Rosemary oil.

Another embodiment comprises cyclosporin A, Lipowax PR®, and Rosemary oil.

Another embodiment comprises cyclosporin A, Lumulse CS-20®, and Rosemary oil.

Another embodiment comprises cyclosporin A, Macol CSA-20®, and Rosemary oil.

Another embodiment comprises cyclosporin A, Marlox B 24/50®, and Rosemary oil.

Another embodiment comprises cyclosporin A, Mazawet 77®, and Rosemary oil.

Another embodiment comprises cyclosporin A, Norfox 1713®, and Rosemary oil.

Another embodiment comprises cyclosporin A, Norfox 2579®, and Rosemary oil.

Another embodiment comprises cyclosporin A, Norfox Lo Foam®, and Rosemary oil.

Another embodiment comprises cyclosporin A, Promulgen D®, and Rosemary oil.

Another embodiment comprises cyclosporin A, Promulgen G®, and Rosemary oil.

Another embodiment comprises cyclosporin A, Renex 30®, and Rosemary oil.

Another embodiment comprises cyclosporin A, Renex 36®, and Rosemary oil.

Another embodiment comprises cyclosporin A, Rhodasun A 24®, and Rosemary oil.

Another embodiment comprises cyclosporin A, Rhodasurf AAE-10®, and Rosemary oil.

Another embodiment comprises cyclosporin A, Rhodasurf BEH-25®, and Rosemary oil.

Another embodiment comprises cyclosporin A, Rhodasurf BEH-40®, and Rosemary oil.

Another embodiment comprises cyclosporin A, Rhodasurf DA 530®, and Rosemary oil.

Another embodiment comprises cyclosporin A, Rhodasurf DA 630®, and Rosemary oil.

Another embodiment comprises cyclosporin A, Rhodasurf DA 639®, and Rosemary oil.

Another embodiment comprises cyclosporin A, Rhodasun LAN-23®, and Rosemary oil.

Another embodiment comprises cyclosporin A, Rhodasurl ON-870®, and Rosemary oil.

Another embodiment comprises cyclosporin A, Rhodasurf ON-877®, and Rosemary oil.

Another embodiment comprises cyclosporin A, Rhodasurf TB-970 FLK®, and Rosemary oil.

Another embodiment comprises cyclosporin A, Ritacet-20®, and Rosemary oil.

Another embodiment comprises cyclosporin A, Ritachol 1000®, and Rosemary oil.

Another embodiment comprises cyclosporin A, Ritachol 2000®, and Rosemary oil.

Another embodiment comprises cyclosporin A, Ritachol 5000®, and Rosemary oil.

Another embodiment comprises cyclosporin A, Ritox 35®, and Rosemary oil.

Another embodiment comprises cyclosporin A, Surfonic DA-4®, and Rosemary oil.

Another embodiment comprises cyclosporin A, Surfonic DA-6®, and Rosemary oil.

Another embodiment comprises cyclosporin A, Surfonic L46-7®, and Rosemary oil.

Another embodiment comprises cyclosporin A, Surfonic POA®, and Rosemary oil.

Another embodiment comprises cyclosporin A, Synthrapol KB®, and Rosemary oil.

Another embodiment comprises cyclosporin A, Teginacid®, and Rosemary oil.

Another embodiment comprises cyclosporin A, Teginacid C®, and Rosemary oil.

Another embodiment comprises cyclosporin A, Tegotens EC 11®, and Rosemary oil.

Another embodiment comprises cyclosporin A, Tinegal NA®, and Rosemary oil.

Another embodiment comprises cyclosporin A, Tomadol 400®, and Rosemary oil.

Another embodiment comprises cyclosporin A, Tomadol 600®, and Rosemary oil.

Another embodiment comprises cyclosporin A, Tomadol 900®, and Rosemary oil.

Another embodiment comprises cyclosporin A, Uniperol O®, and Rosemary oil.

Another embodiment comprises cyclosporin A, Witconol SN Series®, and Rosemary oil.

Another embodiment comprises cyclosporin A, an Ethoxylated Alkylphenol, and Rosemary oil.

Another embodiment comprises cyclosporin A, Antarox LF-222®, and Rosemary oil.

Another embodiment comprises cyclosporin A, Atlox 775®, and Rosemary oil.

Another embodiment comprises cyclosporin A, Caloxylate N-9®, and Rosemary oil.

Another embodiment comprises cyclosporin A, Canasol NF-1000®, and Rosemary oil.

Another embodiment comprises cyclosporin A, Canasbl NF-3000®, and Rosemary oil.

Another embodiment comprises cyclosporin A, Canasol NF-3070®, and Rosemary oil.

Another embodiment comprises cyclosporin A, Canasol OF 1670®, and Rosemary oil.

Another embodiment comprises cyclosporin A, Canasol OF 2570®, and Rosemary oil.

Another embodiment comprises cyclosporin A, Canasol OF 4070®, and Rosemary oil.

Another embodiment comprises cyclosporin A, Chemax DNP-8®, and Rosemary oil.

Another embodiment comprises cyclosporin A, Chemax DNP-18®, and Rosemary oil.

Another embodiment comprises cyclosporin A, Chemax DNP-150/50®, and Rosemary oil.

Another embodiment comprises cyclosporin A, DeSonic 1.5N®, and Rosemary oil.

Another embodiment comprises cyclosporin A, DeSonic 4N®, and Rosemary oil.

Another embodiment comprises cyclosporin A, DeSonic 5N®, and Rosemary oil.

Another embodiment comprises cyclosporin A, DeSonic 6D®, and Rosemary oil.

Another embodiment comprises cyclosporin A, DeSonic 6N®, and Rosemary oil.

Another embodiment comprises cyclosporin A, DeSonic 7N®, and Rosemary oil.

Another embodiment comprises cyclosporin A, DeSonic 9N®, and Rosemary oil.

Another embodiment comprises cyclosporin A, DeSonic 10D®, and Rosemary oil.

Another embodiment comprises cyclosporin A, DeSonic 11N®, and Rosemary oil.

Another embodiment comprises cyclosporin A, DeSonic 12N®, and Rosemary oil.

Another embodiment comprises cyclosporin A, DeSonic 15N®, and Rosemary oil.

Another embodiment comprises cyclosporin A, DeSonic 20N®, and Rosemary oil.

Another embodiment comprises cyclosporin A, Eccoscour RC®, and Rosemary oil.

Another embodiment comprises cyclosporin A, Eccoterge EO-100®, and Rosemary oil.

Another embodiment comprises cyclosporin A, Emulsifier 632/90%®, and Rosemary oil.

Another embodiment comprises cyclosporin A, Geronol AG-821®, and Rosemary oil.

Another embodiment comprises cyclosporin A, Gradonic N-95®, and Rosemary oil.

Another embodiment comprises cyclosporin A, Hetoxide NP-4®, and Rosemary oil.

Another embodiment comprises cyclosporin A, Hetoxide NP-30®, and Rosemary oil.

Another embodiment comprises cyclosporin A, Hostapal N-100®, and Rosemary oil.

Another embodiment comprises cyclosporin A, Hostapal N-110®, and Rosemary oil.

Another embodiment comprises cyclosporin A, Igepal CTA-639W®, and Rosemary oil.

Another embodiment comprises cyclosporin A, Igepal DAP-9®, and Rosemary oil.

Another embodiment comprises cyclosporin A, Igepal OD-410®, and Rosemary oil.

Another embodiment comprises cyclosporin A, Igepal SS-837®, and Rosemary oil.

Another embodiment comprises cyclosporin A, Lipocol NP-9 USP®, and Rosemary oil.

Another embodiment comprises cyclosporin A, Macol DNP-10®, and Rosemary oil.

Another embodiment comprises cyclosporin A, Marlophen NP 5®, and Rosemary oil.

Another embodiment comprises cyclosporin A, Marlophen P 1®, and Rosemary oil.

Another embodiment comprises cyclosporin A, Surfonic NB®, and Rosemary oil.

Another embodiment comprises cyclosporin A, Surfonic OPB-307®, and Rosemary oil.

Another embodiment comprises cyclosporin A, Surfonic OPB-407®, and Rosemary oil.

Another embodiment comprises cyclosporin A, Syn Fac 334®, and Rosemary oil.

Another embodiment comprises cyclosporin A, Syn Fac 8216®, and Rosemary oil.

Another embodiment comprises cyclosporin A, Triton N-57®, and Rosemary oil.

Another embodiment comprises cyclosporin A, Trycol 6956®, and Rosemary oil.

Another embodiment comprises cyclosporin A, Trycol 6961®, and Rosemary oil.

Another embodiment comprises cyclosporin A, Trycol 6964®, and Rosemary oil.

Another embodiment comprises cyclosporin A, Trycol 6969®, and Rosemary oil.

Another embodiment comprises cyclosporin A, Trycol 6974®, and Rosemary oil.

Another embodiment comprises cyclosporin A, Witbreak DRB-127®, and Rosemary oil.

Another embodiment comprises cyclosporin A, Witbreak DRB-127®, and Rosemary oil.

Another embodiment comprises cyclosporin A, Witconol NP Series®, and Rosemary oil.

Another embodiment comprises cyclosporin A, an Ethoxylated Aryl Phenol, and Rosemary oil.

Another embodiment comprises cyclosporin A, Soprophor BSU®, and Rosemary oil.

Another embodiment comprises cyclosporin A, Soprophor CY/8®, and Rosemary oil.

Another embodiment comprises cyclosporin A, Soprophor S/25®, and Rosemary oil.

Another embodiment comprises cyclosporin A, Witconol NIO®, and Rosemary oil.

Another embodiment comprises cyclosporin A, Witconol NIW®, and Rosemary oil.

Another embodiment comprises cyclosporin A, Witconol S-100®, and Rosemary oil.

Another embodiment comprises cyclosporin A, an Ethoxylated Fatty Acid, and Rosemary oil.

Another embodiment comprises cyclosporin A, Aldo PGHMS®, and Rosemary oil.

Another embodiment comprises cyclosporin A, Alkamuls TO-15/HR®, and Rosemary oil.

Another embodiment comprises cyclosporin A, Armotan AL-69-66®, and Rosemary oil.

Another embodiment comprises cyclosporin A, Cerasynt 840®, and Rosemary oil.

Another embodiment comprises cyclosporin A, Cerasynt 945®, and Rosemary oil.

Another embodiment comprises cyclosporin A, Crystal Inhibitor No. 5®, and Rosemary oil.

Another embodiment comprises cyclosporin A, DeThox Acid L-9®, and Rosemary oil.

Another embodiment comprises cyclosporin A, DeThox Acid S-8®, and Rosemary oil.

Another embodiment comprises cyclosporin A, Ethofat 242/25®, and Rosemary oil.

Another embodiment comprises cyclosporin A, Hydropalat 65®, and Rosemary oil.

Another embodiment comprises cyclosporin A, Lipo EGMS®, and Rosemary oil.

Another embodiment comprises cyclosporin A, Lipopeg 2 DL®, and Rosemary oil.

Another embodiment comprises cyclosporin A, Lipopeg 4 DL®, and Rosemary oil.

Another embodiment comprises cyclosporin A, Lipopeg 4-L®, and Rosemary oil.

Another embodiment comprises cyclosporin A, Lipopeg 39-S®, and Rosemary oil.

Another embodiment comprises cyclosporin A, Lipopeg 4-S®, and Rosemary oil.

Another embodiment comprises cyclosporin A, Lipopeg 10-S®, and Rosemary oil.

Another embodiment comprises cyclosporin A, Lipopeg 100-S®, and Rosemary oil.

Another embodiment comprises cyclosporin A, Lipopeg 6000 DS®, and Rosemary oil.

Another embodiment comprises cyclosporin A, Lumulse 40-L®, and Rosemary oil.

Another embodiment comprises cyclosporin A, Lumulse 40-S®, and Rosemary oil.

Another embodiment comprises cyclosporin A, Lumulse 42-L®, and Rosemary oil.

Another embodiment comprises cyclosporin A, Lumulse 42-S®, and Rosemary oil.

Another embodiment comprises cyclosporin A, Lumulse 100-S®, and Rosemary oil.

Another embodiment comprises cyclosporin A, Lumulse 602-S®, and Rosemary oil.

Another embodiment comprises cyclosporin A, Magrabar PGE-20-0®, and Rosemary oil.

Another embodiment comprises cyclosporin A, Magrabar PGE-20L®, and Rosemary oil.

Another embodiment comprises cyclosporin A, Magrabar PGE-20T®, and Rosemary oil.

Another embodiment comprises cyclosporin A, Magrabar PGE-22-0®, and Rosemary oil.

Another embodiment comprises cyclosporin A, Magrabar PGE-22L®, and Rosemary oil.

Another embodiment comprises cyclosporin A, Magrabar PGE-22T®, and Rosemary oil.

Another embodiment comprises cyclosporin A, Magrabar PGE-40-0®, and Rosemary oil.

Another embodiment comprises cyclosporin A, Magrabar PGE-40L®, and Rosemary oil.

Another embodiment comprises cyclosporin A, Magrabar PGE-40T®, and Rosemary oil.

Another embodiment comprises cyclosporin A, Magrabar PGE-42-0®, and Rosemary oil.

Another embodiment comprises cyclosporin A, Magrabar PGE-42L®, and Rosemary oil.

Another embodiment comprises cyclosporin A, Magrabar PGE-42T®, and Rosemary oil.

Another embodiment comprises cyclosporin A, Magrabar PGE-60-0®, and Rosemary oil.

Another embodiment comprises cyclosporin A, Magrabar PGE-60L®, and Rosemary oil.

Another embodiment comprises cyclosporin A, Magrabar PGE-60T®, and Rosemary oil.

Another embodiment comprises cyclosporin A, Magrabar PGE-62-0®, and Rosemary oil.

Another embodiment comprises cyclosporin A, Magrabar PGE-62L®, and Rosemary oil.

Another embodiment comprises cyclosporin A, Magrabar PGE-62T®, and Rosemary oil.

Another embodiment comprises cyclosporin A, Mapeg S-40K®, and Rosemary oil.

Another embodiment comprises cyclosporin A, Marlowet OTS®, and Rosemary oil.

Another embodiment comprises cyclosporin A, Naturechem PGR®, and Rosemary oil.

Another embodiment comprises cyclosporin A, PG No. 4®, and Rosemary oil.

Another embodiment comprises cyclosporin A, Renex 20®, and Rosemary oil.

Another embodiment comprises cyclosporin A, Ritox 52®, and Rosemary oil.

Another embodiment comprises cyclosporin A, Ritox 53®, and Rosemary oil.

Another embodiment comprises cyclosporin A, Ritox 59®, and Rosemary oil.

Another embodiment comprises cyclosporin A, Surfax 8916/A®, and Rosemary oil.

Another embodiment comprises cyclosporin A, Tego Acid S 40 P®, and Rosemary oil.

Another embodiment comprises cyclosporin A, Tego Acid S 100 P®, and Rosemary oil.

Another embodiment comprises cyclosporin A, Tween 20®, and Rosemary oil.

Another embodiment comprises cyclosporin A, Volpo 131®, and Rosemary oil.

Another embodiment comprises cyclosporin A, an Ethoxylated Fatty Ester or Oil, and Rosemary oil.

Another embodiment comprises cyclosporin A, Acconon 6-C10®, and Rosemary oil.

Another embodiment comprises cyclosporin A, Acconon CC-6®, and Rosemary oil.

Another embodiment comprises cyclosporin A, Acconon CO-7®, and Rosemary oil.

Another embodiment comprises cyclosporin A, Aldosperse 40/60 FG®, and Rosemary oil.

Another embodiment comprises cyclosporin A, Aldosperse ML-23®, and Rosemary oil.

Another embodiment comprises cyclosporin A, Aldosperse MS-20 FG®, and Rosemary oil.

Another embodiment comprises cyclosporin A, Alkamuls EL-620®, and Rosemary oil.

Another embodiment comprises cyclosporin A, Alkamuls EL-719®, and Rosemary oil.

Another embodiment comprises cyclosporin A, Alkamuls EL-985®, and Rosemary oil.

Another embodiment comprises cyclosporin A, Arlatone G®, and Rosemary oil.

Another embodiment comprises cyclosporin A, Arlatone T®, and Rosemary oil.

Another embodiment comprises cyclosporin A, Atlas G-1045A®, and Rosemary oil.

Another embodiment comprises cyclosporin A, Atlas G-1086®, and Rosemary oil.

Another embodiment comprises cyclosporin A, Atlas G-1087®, and Rosemary oil.

Another embodiment comprises cyclosporin A, Atlas G-1089®, and Rosemary oil.

Another embodiment comprises cyclosporin A, Atlas G-1096®, and Rosemary oil.

Another embodiment comprises cyclosporin A, Atlas G-1292®, and Rosemary oil.

Another embodiment comprises cyclosporin A, Atlas G-1293®, and Rosemary oil.

Another embodiment comprises cyclosporin A, Atlas G-1300®, and Rosemary oil.

Another embodiment comprises cyclosporin A, Atlas G-7076®, and Rosemary oil.

Another embodiment comprises cyclosporin A, Capmul EMG®, and Rosemary oil.

Another embodiment comprises cyclosporin A, Chemonic CO-40®, and Rosemary oil.

Another embodiment comprises cyclosporin A, Chemonic LI-3®, and Rosemary oil.

Another embodiment comprises cyclosporin A, Chemonic LI-7®, and Rosemary oil.

Another embodiment comprises cyclosporin A, Cirrasol GM®, and Rosemary oil.

Another embodiment comprises cyclosporin A, Cremophor CO 40®, and Rosemary oil.

Another embodiment comprises cyclosporin A, Cremophor CO 410®, and Rosemary oil.

Another embodiment comprises cyclosporin A, Cremophor EL®, and Rosemary oil.

Another embodiment comprises cyclosporin A, Cremophor GC7®, and Rosemary oil.

Another embodiment comprises cyclosporin A, Cremophor RH-40®, and Rosemary oil.

Another embodiment comprises cyclosporin A, Crovol A-40®, and Rosemary oil.

Another embodiment comprises cyclosporin A, Crovol A-70®, and Rosemary oil.

Another embodiment comprises cyclosporin A, Crovol M-70®, and Rosemary oil.

Another embodiment comprises cyclosporin A, Crovol PK-70®, and Rosemary oil.

Another embodiment comprises cyclosporin A, Cutina E-24®, and Rosemary oil.

Another embodiment comprises cyclosporin A, Dacospin 12-R®, and Rosemary oil.

Another embodiment comprises cyclosporin A, Dehymuls HRE-7®, and Rosemary oil.

Another embodiment comprises cyclosporin A, DeSonic 30C®, and Rosemary oil.

Another embodiment comprises cyclosporin A, DeSonic 36C®, and Rosemary oil.

Another embodiment comprises cyclosporin A, DeSonic 40C®, and Rosemary oil.

Another embodiment comprises cyclosporin A, Durfax 60®, and Rosemary oil.

Another embodiment comprises cyclosporin A, Durfax 65®, and Rosemary oil.

Another embodiment comprises cyclosporin A, Durfax 80®, and Rosemary oil.

Another embodiment comprises cyclosporin A, Durfax EOM®, and Rosemary oil.

Another embodiment comprises cyclosporin A, Eccoterge NF-2®, and Rosemary oil.

Another embodiment comprises cyclosporin A, Emulpon CO-360®, and Rosemary oil.

Another embodiment comprises cyclosporin A, Emulpon CO-550®, and Rosemary oil.

Another embodiment comprises cyclosporin A, Emulsogen EL®, and Rosemary oil.

Another embodiment comprises cyclosporin A, Emulsogen HCO 040®, and Rosemary oil.

Another embodiment comprises cyclosporin A, Emulsogen HCO 060®, and Rosemary oil.

Another embodiment comprises cyclosporin A, Emulsynt 1055®, and Rosemary oil.

Another embodiment comprises cyclosporin A, Ethox 3095®, and Rosemary oil.

Another embodiment comprises cyclosporin A, Eumulgin RO-40®, and Rosemary oil.

Another embodiment comprises cyclosporin A, Genapol G-260®, and Rosemary oil.

Another embodiment comprises cyclosporin A, Glycosperse L-20®, and Rosemary oil.

Another embodiment comprises cyclosporin A, Glycosperse O-5®, and Rosemary oil.

Another embodiment comprises cyclosporin A, Glycosperse O-20®, and Rosemary oil.

Another embodiment comprises cyclosporin A, Glycosperse O-20 FG®, and Rosemary oil.

Another embodiment comprises cyclosporin A, Glycosperse S-20®, and Rosemary oil.

Another embodiment comprises cyclosporin A, Glycosperse S-20 FG®, and Rosemary oil.

Another embodiment comprises cyclosporin A, Glycosperse TS-20®, and Rosemary oil.

Another embodiment comprises cyclosporin A, Glycosperse TS-20 FG®, and Rosemary oil.

Another embodiment comprises cyclosporin A, Hetan SL®, and Rosemary oil.

Another embodiment comprises cyclosporin A, Hetan SO®, and Rosemary oil.

Another embodiment comprises cyclosporin A, Hetan SS®, and Rosemary oil.

Another embodiment comprises cyclosporin A, Hetoxide C-2®, and Rosemary oil.

Another embodiment comprises cyclosporin A, Hetoxide C-9®, and Rosemary oil.

Another embodiment comprises cyclosporin A, Hetoxide C-15®, and Rosemary oil.

Another embodiment comprises cyclosporin A, Hetoxide C-25®, and Rosemary oil.

Another embodiment comprises cyclosporin A, Hetoxide C-40®, and Rosemary oil.

Another embodiment comprises cyclosporin A, Hetoxide C-200®, and Rosemary oil.

Another embodiment comprises cyclosporin A, Hetoxide C-200-50%®, and Rosemary oil.

Another embodiment comprises cyclosporin A, Hetoxide GC-30®, and Rosemary oil.

Another embodiment comprises cyclosporin A, Hetoxide HC-60®, and Rosemary oil.

Another embodiment comprises cyclosporin A, Ice No. 2®, and Rosemary oil.

Another embodiment comprises cyclosporin A, Incrocas 30/40®, and Rosemary oil.

Another embodiment comprises cyclosporin A, Lexol EC®, and Rosemary oil.

Another embodiment comprises cyclosporin A, Lexol EO®, and Rosemary oil.

Another embodiment comprises cyclosporin A, Lipocol HCO-40®, and Rosemary oil.

Another embodiment comprises cyclosporin A, Lipocol HCO-60®, and Rosemary oil.

Another embodiment comprises cyclosporin A, Lipocol O-3 Special®, and Rosemary oil.

Another embodiment comprises cyclosporin A, Lipopeg 2-L®, and Rosemary oil.

Another embodiment comprises cyclosporin A, Lipopeg 4-DO®, and Rosemary oil.

Another embodiment comprises cyclosporin A, Lipopeg 4-DS®, and Rosemary oil.

Another embodiment comprises cyclosporin A, Lipovol GTB®, and Rosemary oil.

Another embodiment comprises cyclosporin A, Lonzest SML-20®, and Rosemary oil.

Another embodiment comprises cyclosporin A, Lonzest SMO-20®, and Rosemary oil.

Another embodiment comprises cyclosporin A, Lonzest SMS-20®, and Rosemary oil.

Another embodiment comprises cyclosporin A, Lonzest STO-20®, and Rosemary oil.

Another embodiment comprises cyclosporin A, Lonzest STS-20®, and Rosemary oil.

Another embodiment comprises cyclosporin A, Lumulse GR-40®, and Rosemary oil.

Another embodiment comprises cyclosporin A, Lumulse GRH-40®, and Rosemary oil.

Another embodiment comprises cyclosporin A, Lumulse POE (7) GML®, and Rosemary oil.

Another embodiment comprises cyclosporin A, Lumulse POE (12) Glyc®, and Rosemary oil.

Another embodiment comprises cyclosporin A, Lumulse POE (40) MS KP®, and Rosemary oil.

Another embodiment comprises cyclosporin A, Marlowet 4750®, and Rosemary oil.

Another embodiment comprises cyclosporin A, Marlowet LVS®, and Rosemary oil.

Another embodiment comprises cyclosporin A, Marlowet R 11®, and Rosemary oil.

Another embodiment comprises cyclosporin A, Marlowet R 40®, and Rosemary oil.

Another embodiment comprises cyclosporin A, Mazol 80 MGK®, and Rosemary oil.

Another embodiment comprises cyclosporin A, Nonionic Emulsifier T-9®, and Rosemary oil.

Another embodiment comprises cyclosporin A, Oronal LCG®, and Rosemary oil.

Another embodiment comprises cyclosporin A, Polyderm PPI-CO-200®, and Rosemary oil.

Another embodiment comprises cyclosporin A, Polyderm PPI-CO-40®, and Rosemary oil.

Another embodiment comprises cyclosporin A, Rewoderm LI 520-70®, and Rosemary oil.

Another embodiment comprises cyclosporin A, Ritapeg 150 DS®, and Rosemary oil.

Another embodiment comprises cyclosporin A, Softigen 767®, and Rosemary oil.

Another embodiment comprises cyclosporin A, Surfactol 318®, and Rosemary oil.

Another embodiment comprises cyclosporin A, Surfactol 365®, and Rosemary oil.

Another embodiment comprises cyclosporin A, Syn Lube 107®, and Rosemary oil.

Another embodiment comprises cyclosporin A, Syn Lube 728®, and Rosemary oil.

Another embodiment comprises cyclosporin A, Syn Lube 1632H®, and Rosemary oil.

Another embodiment comprises cyclosporin A, Syn Lube 6277-A®, and Rosemary oil.

Another embodiment comprises cyclosporin A, T-Det C-20®, and Rosemary oil.

Another embodiment comprises cyclosporin A, T-Det C-40®, and Rosemary oil.

Another embodiment comprises cyclosporin A, Tally 100 Plus®, and Rosemary oil.

Another embodiment comprises cyclosporin A, Uniperol EL®, and Rosemary oil.

Another embodiment comprises cyclosporin A, a Fatty Ester, and Rosemary oil.

Another embodiment comprises cyclosporin A, Actralube-Syn 147®, and Rosemary oil.

Another embodiment comprises cyclosporin A, Atlas G-1556®, and Rosemary oil.

Another embodiment comprises cyclosporin A, Atlas G-1564®, and Rosemary oil.

Another embodiment comprises cyclosporin A, Atlasol Base Oil S®, and Rosemary oil.

Another embodiment comprises cyclosporin A, Base ML®, and Rosemary oil.

Another embodiment comprises cyclosporin A, Base MT®, and Rosemary oil.

Another embodiment comprises cyclosporin A, Cerasynt 303®, and Rosemary oil.

Another embodiment comprises cyclosporin A, Dermol 1012®, and Rosemary oil.

Another embodiment comprises cyclosporin A, Kemester 4000®, and Rosemary oil.

Another embodiment comprises cyclosporin A, Lactipol S®, and Rosemary oil.

Another embodiment comprises cyclosporin A, Magrabar PGO®, and Rosemary oil.

Another embodiment comprises cyclosporin A, Mayco Base BFO®, and Rosemary oil.

Another embodiment comprises cyclosporin A, Methyl Linoleate®, and Rosemary oil.

Another embodiment comprises cyclosporin A, Pationic 122A®, and Rosemary oil.

Another embodiment comprises cyclosporin A, Pationic 138C®, and Rosemary oil.

Another embodiment comprises cyclosporin A, Pationic CSL®, and Rosemary oil.

Another embodiment comprises cyclosporin A, Pationic ISL®, and Rosemary oil.

Another embodiment comprises cyclosporin A, Pationic SBL®, and Rosemary oil.

Another embodiment comprises cyclosporin A, Pationic SSL®, and Rosemary oil.

Another embodiment comprises cyclosporin A, Ritasol®, and Rosemary oil.

Another embodiment comprises cyclosporin A, Tego Alkanol CS 20®, and Rosemary oil.

Another embodiment comprises cyclosporin A, Tego Alkanol L23 P®, and Rosemary oil.

Another embodiment comprises cyclosporin A, Tego Alkanol S2®, and Rosemary oil.

Another embodiment comprises cyclosporin A, Tego Alkanol S20 P®, and Rosemary oil.

Another embodiment comprises cyclosporin A, Triemulsifier 600 MS®, and Rosemary oil.

Another embodiment comprises cyclosporin A, a Glycerol Esters, and Rosemary oil.

Another embodiment comprises cyclosporin A, Agro #9 Wint SBO®, and Rosemary oil.

Another embodiment comprises cyclosporin A, Ahcovel Base 700®, and Rosemary oil.

Another embodiment comprises cyclosporin A, Aldo HMS FG®, and Rosemary oil.

Another embodiment comprises cyclosporin A, Aldo MLD®, and Rosemary oil.

Another embodiment comprises cyclosporin A, Aldo MLD FG®, and Rosemary oil.

Another embodiment comprises cyclosporin A, Aldo MO FG®, and Rosemary oil.

Another embodiment comprises cyclosporin A, Aldo MS®, and Rosemary oil.

Another embodiment comprises cyclosporin A, Aldo MS FG®, and Rosemary oil.

Another embodiment comprises cyclosporin A, Aldo MS LG FG®, and Rosemary oil.

Another embodiment comprises cyclosporin A, Aldo MSD®, and Rosemary oil.

Another embodiment comprises cyclosporin A, Aldo MSD FG®, and Rosemary oil.

Another embodiment comprises cyclosporin A, Aldosperse O-20 FG®, and Rosemary oil.

Another embodiment comprises cyclosporin A, Aldosperse TS-20 FG®, and Rosemary oil.

Another embodiment comprises cyclosporin A, Aldosperse TS-40 FG®, and Rosemary oil.

Another embodiment comprises cyclosporin A, Arlacel 165®, and Rosemary oil.

Another embodiment comprises cyclosporin A, Arlacel 186®, and Rosemary oil.

Another embodiment comprises cyclosporin A, Capmul GMO®, and Rosemary oil.

Another embodiment comprises cyclosporin A, Capmul GMS®, and Rosemary oil.

Another embodiment comprises cyclosporin A, Caprol 3GO®, and Rosemary oil.

Another embodiment comprises cyclosporin A, Caprol 3GVS®, and Rosemary oil.

Another embodiment comprises cyclosporin A, Caprol 6G2S®, and Rosemary oil.

Another embodiment comprises cyclosporin A, Caprol 10G40®, and Rosemary oil.

Another embodiment comprises cyclosporin A, Caprol 10G100®, and Rosemary oil.

Another embodiment comprises cyclosporin A, Caprol ET®, and Rosemary oil.

Another embodiment comprises cyclosporin A, Caprol PGE860®, and Rosemary oil.

Another embodiment comprises cyclosporin A, Cerasynt 945®, and Rosemary oil.

Another embodiment comprises cyclosporin A, Cerasynt GMS®, and Rosemary oil.

Another embodiment comprises cyclosporin A, Cerasynt Q®, and Rosemary oil.

Another embodiment comprises cyclosporin A, Cerasynt SD®, and Rosemary oil.

Another embodiment comprises cyclosporin A, Cerasynt WM®, and Rosemary oil.

Another embodiment comprises cyclosporin A, Chemsperse 14®, and Rosemary oil.

Another embodiment comprises cyclosporin A, Cremophor GO-32®, and Rosemary oil.

Another embodiment comprises cyclosporin A, Cremophor GS11®, and Rosemary oil.

Another embodiment comprises cyclosporin A, Cremophor GS-32®, and Rosemary oil.

Another embodiment comprises cyclosporin A, Cutina KD-16®, and Rosemary oil.

Another embodiment comprises cyclosporin A, Dehymuls PGPH®, and Rosemary oil.

Another embodiment comprises cyclosporin A, Dermol DGDIS®, and Rosemary oil.

Another embodiment comprises cyclosporin A, Dermol DGMIS®, and Rosemary oil.

Another embodiment comprises cyclosporin A, Dermol G-76®, and Rosemary oil.

Another embodiment comprises cyclosporin A, Dermol G-7DI®, and Rosemary oil.

Another embodiment comprises cyclosporin A, Dermol NGDI®, and Rosemary oil.

Another embodiment comprises cyclosporin A, Dermolan GLH®, and Rosemary oil.

Another embodiment comprises cyclosporin A, Drewmulse GMO®, and Rosemary oil.

Another embodiment comprises cyclosporin A, Drewpol 3-5-M®, and Rosemary oil.

Another embodiment comprises cyclosporin A, Durlac 100 W®, and Rosemary oil.

Another embodiment comprises cyclosporin A, Dur-Lo®, and Rosemary oil.

Another embodiment comprises cyclosporin A, Dynasan 118®, and Rosemary oil.

Another embodiment comprises cyclosporin A, EC-25®, and Rosemary oil.

Another embodiment comprises cyclosporin A, EM 40®, and Rosemary oil.

Another embodiment comprises cyclosporin A, Emerest 2400®, and Rosemary oil.

Another embodiment comprises cyclosporin A, Emerest 2452®, and Rosemary oil.

Another embodiment comprises cyclosporin A, Empilan G-26®, and Rosemary oil.

Another embodiment comprises cyclosporin A, Genapol TSM®, and Rosemary oil.

Another embodiment comprises cyclosporin A, Hostacerin DGI®, and Rosemary oil.

Another embodiment comprises cyclosporin A, Hostacerin DGL®, and Rosemary oil.

Another embodiment comprises cyclosporin A, Hostacerin DGMS®, and Rosemary oil.

Another embodiment comprises cyclosporin A, Hostacerin DGSB®, and Rosemary oil.

Another embodiment comprises cyclosporin A, Ice No. 2®, and Rosemary oil.

Another embodiment comprises cyclosporin A, lmwitor 742®, and Rosemary oil.

Another embodiment comprises cyclosporin A, lmwitor 780 K®, and Rosemary oil.

Another embodiment comprises cyclosporin A, lmwitor 960 Flakes®, and Rosemary oil.

Another embodiment comprises cyclosporin A, Isolan GI 34®, and Rosemary oil.

Another embodiment comprises cyclosporin A, Isolan GO 33®, and Rosemary oil.

Another embodiment comprises cyclosporin A, Kemester 1000®, and Rosemary oil.

Another embodiment comprises cyclosporin A, Kemester 2000®, and Rosemary oil.

Another embodiment comprises cyclosporin A, Kemester 2000®, and Rosemary oil.

Another embodiment comprises cyclosporin A, Kemester 6000SE®, and Rosemary oil.

Another embodiment comprises cyclosporin A, Lamecreme DGE 18®, and Rosemary oil.

Another embodiment comprises cyclosporin A, Lexemul 515®, and Rosemary oil.

Another embodiment comprises cyclosporin A, Lexemul 561®, and Rosemary oil.

Another embodiment comprises cyclosporin A, Lexemul AR®, and Rosemary oil.

Another embodiment comprises cyclosporin A, Lexemul AS®, and Rosemary oil.

Another embodiment comprises cyclosporin A, Lexemul GDL®, and Rosemary oil.

Another embodiment comprises cyclosporin A, Lexemul T®, and Rosemary oil.

Another embodiment comprises cyclosporin A, Lipomulse 165®, and Rosemary oil.

Another embodiment comprises cyclosporin A, Lumulse GML K®, and Rosemary oil.

Another embodiment comprises cyclosporin A, Lumulse GMO K®, and Rosemary oil.

Another embodiment comprises cyclosporin A, Lumulse GMR K®, and Rosemary oil.

Another embodiment comprises cyclosporin A, Lumulse GMT K®, and Rosemary oil.

Another embodiment comprises cyclosporin A, Magrabar GMC®, and Rosemary oil.

Another embodiment comprises cyclosporin A, Magrabar GMO-CK®, and Rosemary oil.

Another embodiment comprises cyclosporin A, Magrabar MDG-5050®, and Rosemary oil.

Another embodiment comprises cyclosporin A, Magrabar PGO-315®, and Rosemary oil.

Another embodiment comprises cyclosporin A, Magrabar PGO-1010®, and Rosemary oil.

Another embodiment comprises cyclosporin A, Mazol 300K®, and Rosemary oil.

Another embodiment comprises cyclosporin A, Mazol GMO-K®, and Rosemary oil.

Another embodiment comprises cyclosporin A, Mazol GMS-K®, and Rosemary oil.

Another embodiment comprises cyclosporin A, Mazol PG031-K®, and Rosemary oil.

Another embodiment comprises cyclosporin A, Miglyol 812®, and Rosemary oil.

Another embodiment comprises cyclosporin A, Norfox 165C®, and Rosemary oil.

Another embodiment comprises cyclosporin A, Schercemol GMIS®, and Rosemary oil.

Another embodiment comprises cyclosporin A, Tegin®, and Rosemary oil.

Another embodiment comprises cyclosporin A, Tegin 4100 Pellets®, and Rosemary oil.

Another embodiment comprises cyclosporin A, Tegin M Pellets®, and Rosemary oil.

Another embodiment comprises cyclosporin A, Tegin OV®, and Rosemary oil.

Another embodiment comprises cyclosporin A, Teginacid H®, and Rosemary oil.

Another embodiment comprises cyclosporin A, Tego Cosmo P813®, and Rosemary oil.

Another embodiment comprises cyclosporin A, Wickenol 535®, and Rosemary oil.

Another embodiment comprises cyclosporin A, Witconol 14®, and Rosemary oil.

Another embodiment comprises cyclosporin A, Witconol 14®, and Rosemary oil.

Another embodiment comprises cyclosporin A, Witconol 14F®, and Rosemary oil.

Another embodiment comprises cyclosporin A, Witconol 18L®, and Rosemary oil.

Another embodiment comprises cyclosporin A, Witconol GOT®, and Rosemary oil.

Another embodiment comprises cyclosporin A, Witconol MST®, and Rosemary oil.

Another embodiment comprises cyclosporin A, Witconol RHT®, and Rosemary oil.

Another embodiment comprises cyclosporin A, a Glycol Ester, and Rosemary oil.

Another embodiment comprises cyclosporin A, Alkamuls 600 DO®, and Rosemary oil.

Another embodiment comprises cyclosporin A, Alkamuls SEG®, and Rosemary oil.

Another embodiment comprises cyclosporin A, Atlas EM-2®, and Rosemary oil.

Another embodiment comprises cyclosporin A, Cerasynt IP®, and Rosemary oil.

Another embodiment comprises cyclosporin A, Cerasynt M®, and Rosemary oil.

Another embodiment comprises cyclosporin A, Cerasynt MN®, and Rosemary oil.

Another embodiment comprises cyclosporin A, Cerasynt PA®, and Rosemary oil.

Another embodiment comprises cyclosporin A, Chemsperse EGDS®, and Rosemary oil.

Another embodiment comprises cyclosporin A, Chemsperse EGMS®, and Rosemary oil.

Another embodiment comprises cyclosporin A, Colonial Monolaurin®, and Rosemary oil.

Another embodiment comprises cyclosporin A, DeMuls SGE-95®, and Rosemary oil.

Another embodiment comprises cyclosporin A, Eccoterge 200®, and Rosemary oil.

Another embodiment comprises cyclosporin A, Emerest 2380®, and Rosemary oil.

Another embodiment comprises cyclosporin A, Ethox 2610®, and Rosemary oil.

Another embodiment comprises cyclosporin A, Ethox DO-9®, and Rosemary oil.

Another embodiment comprises cyclosporin A, Ethox DO-14®, and Rosemary oil.

Another embodiment comprises cyclosporin A, Ethox SO-9®, and Rosemary oil.

Another embodiment comprises cyclosporin A, Fizul MD-318®, and Rosemary oil.

Another embodiment comprises cyclosporin A, Genapol EGDS-VHP®, and Rosemary oil.

Another embodiment comprises cyclosporin A, Genapol TS Powder®, and Rosemary oil.

Another embodiment comprises cyclosporin A, Hostacerin WO®, and Rosemary oil.

Another embodiment comprises cyclosporin A, Inversol 140®, and Rosemary oil.

Another embodiment comprises cyclosporin A, Kemester 104®, and Rosemary oil.

Another embodiment comprises cyclosporin A, Kemester 205®, and Rosemary oil.

Another embodiment comprises cyclosporin A, Kemester 226®, and Rosemary oil.

Another embodiment comprises cyclosporin A, Kemester 5221SE®, and Rosemary oil.

Another embodiment comprises cyclosporin A, Kemester EGDS®, and Rosemary oil.

Another embodiment comprises cyclosporin A, Lexemul EGDS®, and Rosemary oil.

Another embodiment comprises cyclosporin A, Lexemul EGMS®, and Rosemary oil.

Another embodiment comprises cyclosporin A, Lexemul P®, and Rosemary oil.

Another embodiment comprises cyclosporin A, Lipo DGLS®, and Rosemary oil.

Another embodiment comprises cyclosporin A, Lipo EGDS®, and Rosemary oil.

Another embodiment comprises cyclosporin A, Lipo PGMS®, and Rosemary oil.

Another embodiment comprises cyclosporin A, Liposorb S-4®, and Rosemary oil.

Another embodiment comprises cyclosporin A, Liposorb TO-20®, and Rosemary oil.

Another embodiment comprises cyclosporin A, Lumulse PGO®, and Rosemary oil.

Another embodiment comprises cyclosporin A, Mackester EGDS®, and Rosemary oil.

Another embodiment comprises cyclosporin A, Mackester EGMS®, and Rosemary oil.

Another embodiment comprises cyclosporin A, Mackester GSTP®, and Rosemary oil.

Another embodiment comprises cyclosporin A, Mackester Series®, and Rosemary oil.

Another embodiment comprises cyclosporin A, Magrabar PDG-50®, and Rosemary oil.

Another embodiment comprises cyclosporin A, Mapeg 6000 DS®, and Rosemary oil.

Another embodiment comprises cyclosporin A, Marlowet 4702®, and Rosemary oil.

Another embodiment comprises cyclosporin A, Monalube 305®, and Rosemary oil.

Another embodiment comprises cyclosporin A, Monalube 310®, and Rosemary oil.

Another embodiment comprises cyclosporin A, Monalube 315®, and Rosemary oil.

Another embodiment comprises cyclosporin A, Monalube 320®, and Rosemary oil.

Another embodiment comprises cyclosporin A, Monalube 325®, and Rosemary oil.

Another embodiment comprises cyclosporin A, Monalube 330®, and Rosemary oil.

Another embodiment comprises cyclosporin A, Naturechem PGHS®, and Rosemary oil.

Another embodiment comprises cyclosporin A, Polycastorol PLO-840®, and Rosemary oil.

Another embodiment comprises cyclosporin A, Polytex 10OM®, and Rosemary oil.

Another embodiment comprises cyclosporin A, Ritasynt IP®, and Rosemary oil.

Another embodiment comprises cyclosporin A, Ross Chem PEG 600 DT®, and Rosemary oil.

Another embodiment comprises cyclosporin A, Schercemol PGMS®, and Rosemary oil.

Another embodiment comprises cyclosporin A, Sponto H-44C®, and Rosemary oil.

Another embodiment comprises cyclosporin A, Tegin G®, and Rosemary oil.

Another embodiment comprises cyclosporin A, Witbreak DGE-182®, and Rosemary oil.

Another embodiment comprises cyclosporin A, Witbreak DGE-182®, and Rosemary oil.

Another embodiment comprises cyclosporin A, Witbreak DRA-21®, and Rosemary oil.

Another embodiment comprises cyclosporin A, Witbreak DRA-21®, and Rosemary oil.

Another embodiment comprises cyclosporin A, Witbreak DRA-50®, and Rosemary oil.

Another embodiment comprises cyclosporin A, Witbreak DRA-50®, and Rosemary oil.

Another embodiment comprises cyclosporin A, Witconol F26-46®, and Rosemary oil.

Another embodiment comprises cyclosporin A, Witconol H-32®, and Rosemary oil.

Another embodiment comprises cyclosporin A, Witconol H-33®, and Rosemary oil.

Another embodiment comprises cyclosporin A, Witconol H-35A®, and Rosemary oil.

Another embodiment comprises cyclosporin A, Witconol RHP®, and Rosemary oil.

Another embodiment comprises cyclosporin A, a Lanolin-based Derivative, and Rosemary oil.

Another embodiment comprises cyclosporin A, Amerchol CAB®, and Rosemary oil.

Another embodiment comprises cyclosporin A, Amerchol L-101®, and Rosemary oil.

Another embodiment comprises cyclosporin A, Amerlate LFA-LO®, and Rosemary oil.

Another embodiment comprises cyclosporin A, Amerlate P®, and Rosemary oil.

Another embodiment comprises cyclosporin A, Barre Common Degras®, and Rosemary oil.

Another embodiment comprises cyclosporin A, Cholesterol NF®, and Rosemary oil.

Another embodiment comprises cyclosporin A, Crodalan AWS®, and Rosemary oil.

Another embodiment comprises cyclosporin A, Crodalan LA®, and Rosemary oil.

Another embodiment comprises cyclosporin A, Emery 1650®, and Rosemary oil.

Another embodiment comprises cyclosporin A, Emery 1650®, and Rosemary oil.

Another embodiment comprises cyclosporin A, Emery 1740®, and Rosemary oil.

Another embodiment comprises cyclosporin A, Emery 1740®, and Rosemary oil.

Another embodiment comprises cyclosporin A, Forlan 500®, and Rosemary oil.

Another embodiment comprises cyclosporin A, Forlan L®, and Rosemary oil.

Another embodiment comprises cyclosporin A, Laneto 50®, and Rosemary oil.

Another embodiment comprises cyclosporin A, Laneto 100®, and Rosemary oil.

Another embodiment comprises cyclosporin A, Laneto AWS®, and Rosemary oil.

Another embodiment comprises cyclosporin A, Lanfrax 1776®, and Rosemary oil.

Another embodiment comprises cyclosporin A, Lanfrax 1776®, and Rosemary oil.

Another embodiment comprises cyclosporin A, Lanogel 21®, and Rosemary oil.

Another embodiment comprises cyclosporin A, Lipolan®, and Rosemary oil.

Another embodiment comprises cyclosporin A, Lipolan 31®, and Rosemary oil.

Another embodiment comprises cyclosporin A, OHlan®, and-Rosemary oil.

Another embodiment comprises cyclosporin A, Polychol 5®, and Rosemary oil.

Another embodiment comprises cyclosporin A, Polychol 15®, and Rosemary oil.

Another embodiment comprises cyclosporin A, Ritacetyl®, and Rosemary oil.

Another embodiment comprises cyclosporin A, Ritachol®, and Rosemary oil.

Another embodiment comprises cyclosporin A, Ritahydrox®, and Rosemary oil.

Another embodiment comprises cyclosporin A, Ritalafa®, and Rosemary oil.

Another embodiment comprises cyclosporin A, Ritalan®, and Rosemary oil.

Another embodiment comprises cyclosporin A, Ritalan AWS®, and Rosemary oil.

Another embodiment comprises cyclosporin A, Ritalan C®, and Rosemary oil.

Another embodiment comprises cyclosporin A, Ritawax®, and Rosemary oil.

Another embodiment comprises cyclosporin A, Ritawax AEO®, and Rosemary oil.

Another embodiment comprises cyclosporin A, Ritawax ALA®, and Rosemary oil.

Another embodiment comprises cyclosporin A, Solan/Solan 50/Super Solan®, and Rosemary oil.

Another embodiment comprises cyclosporin A, Super Hartolan/Hartolan®, and Rosemary oil.

Another embodiment comprises cyclosporin A, Supersat AWS-4®, and Rosemary oil.

Another embodiment comprises cyclosporin A, Supersat AWS-24®, and Rosemary oil.

Another embodiment comprises cyclosporin A, a Lecithin or Lecithin Derivative, and Rosemary oil.

Another embodiment comprises cyclosporin A, Alcolec®, and Rosemary oil.

Another embodiment comprises cyclosporin A, Lecithin®, and Rosemary oil.

Another embodiment comprises cyclosporin A, Lexin K®, and Rosemary oil.

Another embodiment comprises cyclosporin A, Natipide®, and Rosemary oil.

Another embodiment comprises cyclosporin A, a Lignin or Lignin Derivative, and Rosemary oil.

Another embodiment comprises cyclosporin A, Diwatex XP 9®, and Rosemary oil.

Another embodiment comprises cyclosporin A, Dynasperse LCD®, and Rosemary oil.

Another embodiment comprises cyclosporin A, Indulin SAL®, and Rosemary oil.

Another embodiment comprises cyclosporin A, Indulin W-1®, and Rosemary oil.

Another embodiment comprises cyclosporin A, Indulin W-5®, and Rosemary oil.

Another embodiment comprises cyclosporin A, Lignosol FTA®, and Rosemary oil.

Another embodiment comprises cyclosporin A, Lignosol SFX-65®, and Rosemary oil.

Another embodiment comprises cyclosporin A, Marasperse 52 CP®, and Rosemary oil.

Another embodiment comprises cyclosporin A, Marasperse AG®, and Rosemary oil.

Another embodiment comprises cyclosporin A, Marasperse CBOS-4®, and Rosemary oil.

Another embodiment comprises cyclosporin A, Ufoxane 2®, and Rosemary oil.

Another embodiment comprises cyclosporin A, a Methyl Ester, and Rosemary oil.

Another embodiment comprises cyclosporin A, E.B. Cleaner AK®, and Rosemary oil.

Another embodiment comprises cyclosporin A, Oleocal ME-70®, and Rosemary oil.

Another embodiment comprises cyclosporin A, Trade Name Company®, and Rosemary oil.

Another embodiment comprises cyclosporin A, Methyl Esters (cont'd)®, and Rosemary oil.

Another embodiment comprises cyclosporin A, Oleocal ME-92®, and Rosemary oil.

Another embodiment comprises cyclosporin A, Oleocal ME-112®, and Rosemary oil.

Another embodiment comprises cyclosporin A, Oleocal ME-130®, and Rosemary oil.

Another embodiment comprises cyclosporin A, a Monoglyceride or a Derivative thereof, and Rosemary oil.

Another embodiment comprises cyclosporin A, Dynacet 211®, and Rosemary oil.

Another embodiment comprises cyclosporin A, Hetsorb S-20®, and Rosemary oil.

Another embodiment comprises cyclosporin A, lmwitor 191®, and Rosemary oil.

Another embodiment comprises cyclosporin A, lmwitor 370®, and Rosemary oil.

Another embodiment comprises cyclosporin A, lmwitor 375®, and Rosemary oil.

Another embodiment comprises cyclosporin A, lmwitor 900®, and Rosemary oil.

Another embodiment comprises cyclosporin A, Imwitor 945®, and Rosemary oil.

Another embodiment comprises cyclosporin A, Imwitor 2020®, and Rosemary oil.

Another embodiment comprises cyclosporin A, Kemester 5500®, and Rosemary oil.

Another embodiment comprises cyclosporin A, Kemester 6000®, and Rosemary oil.

Another embodiment comprises cyclosporin A, Magrabar GMC®, and Rosemary oil.

Another embodiment comprises cyclosporin A, Magrabar GMO-CK®, and Rosemary oil.

Another embodiment comprises cyclosporin A, Magrabar GPC-10®, and Rosemary oil.

Another embodiment comprises cyclosporin A, Magrabar MDG-5050®, and Rosemary oil.

Another embodiment comprises cyclosporin A, Monalube 335®, and Rosemary oil.

Another embodiment comprises cyclosporin A, Monoglycerides Glyceryl Monestearate Archer Daniels Midland Company®, and Rosemary oil.

Another embodiment comprises cyclosporin A, Rita GMS®, and Rosemary oil.

Another embodiment comprises cyclosporin A, Ritamulse SCG®, and Rosemary oil.

Another embodiment comprises cyclosporin A, Softigen 701®, and Rosemary oil.

Another embodiment comprises cyclosporin A, Tally 100 Plus®, and Rosemary oil.

Another embodiment comprises cyclosporin A, a Polyethylene Glycol, and Rosemary oil.

Another embodiment comprises cyclosporin A, Emulgade PL 68/50®, and Rosemary oil.

Another embodiment comprises cyclosporin A, Lumulse PEG®, and Rosemary oil.

Another embodiment comprises cyclosporin A, Rhodasun PEG-400®, and Rosemary oil.

Another embodiment comprises cyclosporin A, Rhodasurt PEG-600®, and Rosemary oil.

Another embodiment comprises cyclosporin A, Witconol PEG-400®, and Rosemary oil.

Another embodiment comprises cyclosporin A, a Polymeric Surfactant, and Rosemary oil.

Another embodiment comprises cyclosporin A, Acritamer PNC-EG®, and Rosemary oil.

Another embodiment comprises cyclosporin A, Ag-Rho DEP-775®, and Rosemary oil.

Another embodiment comprises cyclosporin A, APG 325N Glycoside®, and Rosemary oil.

Another embodiment comprises cyclosporin A, Aristoflex AVC®, and Rosemary oil.

Another embodiment comprises cyclosporin A, Aristoflex HMB®, and Rosemary oil.

Another embodiment comprises cyclosporin A, Burco NPS-225®, and Rosemary oil.

Another embodiment comprises cyclosporin A, Burco NPS-816®, and Rosemary oil.

Another embodiment comprises cyclosporin A, Chemccinate 5603®, and Rosemary oil.

Another embodiment comprises cyclosporin A, Cosmedia Guar C-261N®, and Rosemary oil.

Another embodiment comprises cyclosporin A, Gantrez S-95®, and Rosemary oil.

Another embodiment comprises cyclosporin A, Glucopon 220 UP®, and Rosemary oil.

Another embodiment comprises cyclosporin A, Glucopon 225 DK®, and Rosemary oil.

Another embodiment comprises cyclosporin A, Glucopon 425 N®, and Rosemary oil.

Another embodiment comprises cyclosporin A, Glucopon 600 UP®, and Rosemary oil.

Another embodiment comprises cyclosporin A, Glucopon 625 UP®, and Rosemary oil.

Another embodiment comprises cyclosporin A, Pemulen 1621®, and Rosemary oil.

Another embodiment comprises cyclosporin A, Pemulen 1622®, and Rosemary oil.

Another embodiment comprises cyclosporin A, Pemulen TR-1®, and Rosemary oil.

Another embodiment comprises cyclosporin A, Pemulen TR-2®, and Rosemary oil.

Another embodiment comprises cyclosporin A, Plantacare 818®, and Rosemary oil.

Another embodiment comprises cyclosporin A, Plantapon LGC Sorb®, and Rosemary oil.

Another embodiment comprises cyclosporin A, Plantaren 1200N®, and Rosemary oil.

Another embodiment comprises cyclosporin A, Plantaren 2000N®, and Rosemary oil.

Another embodiment comprises cyclosporin A, Viscolam AT 64®, and Rosemary oil.

Another embodiment comprises cyclosporin A, Viscolam AT 64P®, and Rosemary oil.

Another embodiment comprises cyclosporin A, Viscolam AT 100®, and Rosemary oil.

Another embodiment comprises cyclosporin A, Viscolam MAC 7®, and Rosemary oil.

Another embodiment comprises cyclosporin A, Viscolam SMC 20®, and Rosemary oil.

Another embodiment comprises cyclosporin A, Witbreak RTC-323®, and Rosemary oil.

Another embodiment comprises cyclosporin A, WSI 3700®, and Rosemary oil.

Another embodiment comprises cyclosporin A, a Propoxylated or Ethoxylated Fatty Acid, Alcohol, or Alkyl Phenol, and Rosemary oil.

Another embodiment comprises cyclosporin A, Antarox AA-60®, and Rosemary oil.

Another embodiment comprises cyclosporin A, Antarox LF-224®, and Rosemary oil.

Another embodiment comprises cyclosporin A, Burcomul DFE-45®, and Rosemary oil.

Another embodiment comprises cyclosporin A, Burcoterge LFE-1000®, and Rosemary oil.

Another embodiment comprises cyclosporin A, Chemal LF-25B®, and Rosemary oil.

Another embodiment comprises cyclosporin A, Chemal LF-40B®, and Rosemary oil.

Another embodiment comprises cyclosporin A, Dehypon LS-36®, and Rosemary oil.

Another embodiment comprises cyclosporin A, Dehypon LS-36®, and Rosemary oil.

Another embodiment comprises cyclosporin A, Dehypon LS-54®, and Rosemary oil.

Another embodiment comprises cyclosporin A, Dehypon LS-54®, and Rosemary oil.

Another embodiment comprises cyclosporin A, Delonic 100 VLF®, and Rosemary oil.

Another embodiment comprises cyclosporin A, Delonic LF-60 MOD®, and Rosemary oil.

Another embodiment comprises cyclosporin A, Empiderm B®, and Rosemary oil.

Another embodiment comprises cyclosporin A, Ethylan 1206®, and Rosemary oil.

Another embodiment comprises cyclosporin A, Ethylan NS-500K®, and Rosemary oil.

Another embodiment comprises cyclosporin A, Ethylan NS-500LQ®, and Rosemary oil.

Another embodiment comprises cyclosporin A, Genapol 1392®, and Rosemary oil.

Another embodiment comprises cyclosporin A, Genapol 2317®, and Rosemary oil.

Another embodiment comprises cyclosporin A, Genapol 26EP710®, and Rosemary oil.

Another embodiment comprises cyclosporin A, Genapol EP 1022®, and Rosemary oil.

Another embodiment comprises cyclosporin A, Genapol EP 1024®, and Rosemary oil.

Another embodiment comprises cyclosporin A, Genapol EP 6068®, and Rosemary oil.

Another embodiment comprises cyclosporin A, Genapol NP915®, and Rosemary oil.

Another embodiment comprises cyclosporin A, Kieralon MFB®, and Rosemary oil.

Another embodiment comprises cyclosporin A, Lumisolve CSA-80 V®, and Rosemary oil.

Another embodiment comprises cyclosporin A, Marlowet 5001®, and Rosemary oil.

Another embodiment comprises cyclosporin A, Marlox FK 64®, and Rosemary oil.

Another embodiment comprises cyclosporin A, Marlox MO 124®, and Rosemary oil.

Another embodiment comprises cyclosporin A, Marlox S 58®, and Rosemary oil.

Another embodiment comprises cyclosporin A, Nonatell 1003®, and Rosemary oil.

Another embodiment comprises cyclosporin A, Nonatell 1038®, and Rosemary oil.

Another embodiment comprises cyclosporin A, Nonatell 1052®, and Rosemary oil.

Another embodiment comprises cyclosporin A, Nonatell 1061®, and Rosemary oil.

Another embodiment comprises cyclosporin A, Nonatell 1075®, and Rosemary oil.

Another embodiment comprises cyclosporin A, Nonatell 1088®, and Rosemary oil.

Another embodiment comprises cyclosporin A, Nonatell 1123®, and Rosemary oil.

Another embodiment comprises cyclosporin A, Nonatell 1153®, and Rosemary oil.

Another embodiment comprises cyclosporin A, Nonatell 1161®, and Rosemary oil.

Another embodiment comprises cyclosporin A, Nonatell 1172®, and Rosemary oil.

Another embodiment comprises cyclosporin A, Nonatell 1181®, and Rosemary oil.

Another embodiment comprises cyclosporin A, Norfox 36®, and Rosemary oil.

Another embodiment comprises cyclosporin A, Procetyl AWS®, and Rosemary oil.

Another embodiment comprises cyclosporin A, Sandoxylate SX 412®, and Rosemary oil.

Another embodiment comprises cyclosporin A, Sandoxylate SX 418®, and Rosemary oil.

Another embodiment comprises cyclosporin A, Surfonic JL-80X®, and Rosemary oil.

Another embodiment comprises cyclosporin A, Surfonic JL-80X-B1®, and Rosemary oil.

Another embodiment comprises cyclosporin A, Surfonic L4-29X®, and Rosemary oil.

Another embodiment comprises cyclosporin A, Surfonic LF®, and Rosemary oil.

Another embodiment comprises cyclosporin A, T-Det A826®, and Rosemary oil.

Another embodiment comprises cyclosporin A, T-Det LF-416®, and Rosemary oil.

Another embodiment comprises cyclosporin A, Tergitol Min-Foam 1X®, and Rosemary oil.

Another embodiment comprises cyclosporin A, Tergitol Min-Foam 2X®, and Rosemary oil.

Another embodiment comprises cyclosporin A, Triton CF-21®, and Rosemary oil.

Another embodiment comprises cyclosporin A, Triton CF-76®, and Rosemary oil.

Another embodiment comprises cyclosporin A, Triton XL-80N®, and Rosemary oil.

Another embodiment comprises cyclosporin A, Witconol NS-98®, and Rosemary oil.

Another embodiment comprises cyclosporin A, Witconol NS-108LQ®, and Rosemary oil.

Another embodiment comprises cyclosporin A, Witconol NS-145®, and Rosemary oil.

Another embodiment comprises cyclosporin A, Witconol NS-179®, and Rosemary oil.

Another embodiment comprises cyclosporin A, a Protein-based Surfactant, and Rosemary oil.

Another embodiment comprises cyclosporin A, AminoFoam W®, and Rosemary oil.

Another embodiment comprises cyclosporin A, Amiter LGOD-2®, and Rosemary oil.

Another embodiment comprises cyclosporin A, Amiter LGS-2®, and Rosemary oil.

Another embodiment comprises cyclosporin A, Amiter LGS-5®, and Rosemary oil.

Another embodiment comprises cyclosporin A, Lamepon S®, and Rosemary oil.

Another embodiment comprises cyclosporin A, Lamepon S®, and Rosemary oil.

Another embodiment comprises cyclosporin A, Maypon 4C®, and Rosemary oil.

Another embodiment comprises cyclosporin A, May-Tein C®, and Rosemary oil.

Another embodiment comprises cyclosporin A, May-Tein CT®, and Rosemary oil.

Another embodiment comprises cyclosporin A, May-Tein KTS®, and Rosemary oil.

Another embodiment comprises cyclosporin A, May-Tein SY®, and Rosemary oil.

Another embodiment comprises cyclosporin A, Plantapon S®, and Rosemary oil.

Another embodiment comprises cyclosporin A, Proteol APL®, and Rosemary oil.

Another embodiment comprises cyclosporin A, Proteol OAT®, and Rosemary oil.

Another embodiment comprises cyclosporin A, Pyroter CPI-40®, and Rosemary oil.

Another embodiment comprises cyclosporin A, Pyroter GPI-25®, and Rosemary oil.

Another embodiment comprises cyclosporin A, Supro-Tein S®, and Rosemary oil.

Another embodiment comprises cyclosporin A, Supro-Tein V®, and Rosemary oil.

Another embodiment comprises cyclosporin A,® and Rosemary oil.

Another embodiment comprises cyclosporin A, a Sarcosine Derivative®, and Rosemary oil.

Another embodiment comprises cyclosporin A, Crodasinic LS-30®, and Rosemary oil.

Another embodiment comprises cyclosporin A, Vanseal CS®, and Rosemary oil.

Another embodiment comprises cyclosporin A, Vanseal LS®, and Rosemary oil.

Another embodiment comprises cyclosporin A, Vanseal MS®, and Rosemary oil.

Another embodiment comprises cyclosporin A, Vanseal NACS-30®, and Rosemary oil.

Another embodiment comprises cyclosporin A, Vanseal NALS-95®, and Rosemary oil.

Another embodiment comprises cyclosporin A, Vanseal OS®, and Rosemary oil.

Another embodiment comprises cyclosporin A,® and Rosemary oil.

Another embodiment comprises cyclosporin A, a Silicone-based Surfactant®, and Rosemary oil.

Another embodiment comprises cyclosporin A, Abil-B-9950®, and Rosemary oil.

Another embodiment comprises cyclosporin A, Abil Care 85®, and Rosemary oil.

Another embodiment comprises cyclosporin A, Abil EM 90®, and Rosemary oil.

Another embodiment comprises cyclosporin A, Abil EM 97®, and Rosemary oil.

Another embodiment comprises cyclosporin A, Abil WE-09®, and Rosemary oil.

Another embodiment comprises cyclosporin A, Dow Corning 1248 Fluid®, and Rosemary oil.

Another embodiment comprises cyclosporin A, Dow Corning 3225C®, and Rosemary oil.

Another embodiment comprises cyclosporin A, Dow Corning 5200®, and Rosemary oil.

Another embodiment comprises cyclosporin A, Dow Corning Q4-3667®, and Rosemary oil.

Another embodiment comprises cyclosporin A, Monasil PCA®, and Rosemary oil.

Another embodiment comprises cyclosporin A, Monasil PDM®, and Rosemary oil.

Another embodiment comprises cyclosporin A, Monasil PLN®, and Rosemary oil.

Another embodiment comprises cyclosporin A, Polyderm PPI-SI-WS®, and Rosemary oil.

Another embodiment comprises cyclosporin A, Troysol 380W®, and Rosemary oil.

Another embodiment comprises cyclosporin A, Troysol S366®, and Rosemary oil.

Another embodiment comprises cyclosporin A, a Sorbitan Derivative, and Rosemary oil.

Another embodiment comprises cyclosporin A, Alkamuls SML®, and Rosemary oil.

Another embodiment comprises cyclosporin A, Alkamuls SMO®, and Rosemary oil.

Another embodiment comprises cyclosporin A, Alkamuls STO®, and Rosemary oil.

Another embodiment comprises cyclosporin A, Arlacel 20®, and Rosemary oil.

Another embodiment comprises cyclosporin A, Arlacel 40®, and Rosemary oil.

Another embodiment comprises cyclosporin A, Arlacel 60®, and Rosemary oil.

Another embodiment comprises cyclosporin A, Arlacel 80®, and Rosemary oil.

Another embodiment comprises cyclosporin A, Arlacel C®, and Rosemary oil.

Another embodiment comprises cyclosporin A, Armul 21®, and Rosemary oil.

Another embodiment comprises cyclosporin A, Atlox 80®, and Rosemary oil.

Another embodiment comprises cyclosporin A, Atlox 847®, and Rosemary oil.

Another embodiment comprises cyclosporin A, Atlox 1045A®, and Rosemary oil.

Another embodiment comprises cyclosporin A, Canarcel 20®, and Rosemary oil.

Another embodiment comprises cyclosporin A, Canarcel 60®, and Rosemary oil.

Another embodiment comprises cyclosporin A, Canarcel 80®, and Rosemary oil.

Another embodiment comprises cyclosporin A, Canarcel TW 20®, and Rosemary oil.

Another embodiment comprises cyclosporin A, Canarcel TW 60®, and Rosemary oil.

Another embodiment comprises cyclosporin A, Canarcel TW 80®, and Rosemary oil.

Another embodiment comprises cyclosporin A, Coladet BSB-P®, and Rosemary oil.

Another embodiment comprises cyclosporin A, Customulse O-20®, and Rosemary oil.

Another embodiment comprises cyclosporin A, Dehymuls E®, and Rosemary oil.

Another embodiment comprises cyclosporin A, DeSotan SMO®, and Rosemary oil.

Another embodiment comprises cyclosporin A, DeSotan SMO-20®, and Rosemary oil.

Another embodiment comprises cyclosporin A, DeSotan SMT®, and Rosemary oil.

Another embodiment comprises cyclosporin A, DeSotan SMT-20®, and Rosemary oil.

Another embodiment comprises cyclosporin A, Dudax 60®, and Rosemary oil.

Another embodiment comprises cyclosporin A, Durtax 65®, and Rosemary oil.

Another embodiment comprises cyclosporin A, Durfax 80®, and Rosemary oil.

Another embodiment comprises cyclosporin A, Durtan 60®, and Rosemary oil.

Another embodiment comprises cyclosporin A, Durtan 65®, and Rosemary oil.

Another embodiment comprises cyclosporin A, Liposorb L®, and Rosemary oil.

Another embodiment comprises cyclosporin A, Liposorb L-10®, and Rosemary oil.

Another embodiment comprises cyclosporin A, Liposorb L-20®, and Rosemary oil.

Another embodiment comprises cyclosporin A, Liposorb O®, and Rosemary oil.

Another embodiment comprises cyclosporin A, Liposorb O-20®, and Rosemary oil.

Another embodiment comprises cyclosporin A, Liposorb P®, and Rosemary oil.

Another embodiment comprises cyclosporin A, Liposorb P-20®, and Rosemary oil.

Another embodiment comprises cyclosporin A, Liposorb S®, and Rosemary oil.

Another embodiment comprises cyclosporin A, Liposorb S-20®, and Rosemary oil.

Another embodiment comprises cyclosporin A, Liposorb SQO®, and Rosemary oil.

Another embodiment comprises cyclosporin A, Liposorb TO®, and Rosemary oil.

Another embodiment comprises cyclosporin A, Liposorb TS®, and Rosemary oil.

Another embodiment comprises cyclosporin A, Liposorb TS-20®, and Rosemary oil.

Another embodiment comprises cyclosporin A, Lumisorb PS®, and Rosemary oil.

Another embodiment comprises cyclosporin A, Lumisorb SMO (T)®, and Rosemary oil.

Another embodiment comprises cyclosporin A, Lumisorb SMS K®, and Rosemary oil.

Another embodiment comprises cyclosporin A, Lumisorb SSO®, and Rosemary oil.

Another embodiment comprises cyclosporin A, Lumisorb STS K®, and Rosemary oil.

Another embodiment comprises cyclosporin A, Lumisorb STT®, and Rosemary oil.

Another embodiment comprises cyclosporin A, Magrabar SMO®, and Rosemary oil.

Another embodiment comprises cyclosporin A, Magrabar SMO-VEG®, and Rosemary oil.

Another embodiment comprises cyclosporin A, Magrabar SMT®, and Rosemary oil.

Another embodiment comprises cyclosporin A, Magrabar STO®, and Rosemary oil.

Another embodiment comprises cyclosporin A, Miracare BC-27®, and Rosemary oil.

Another embodiment comprises cyclosporin A, Ritabate 20®, and Rosemary oil.

Another embodiment comprises cyclosporin A, Ritabate 40®, and Rosemary oil.

Another embodiment comprises cyclosporin A, Ritabate 60®, and Rosemary oil.

Another embodiment comprises cyclosporin A, Ritabate 80®, and Rosemary oil.

Another embodiment comprises cyclosporin A, T-Maz®, and Rosemary oil.

Another embodiment comprises cyclosporin A, Tego SML®, and Rosemary oil.

Another embodiment comprises cyclosporin A, Tego SML 20®, and Rosemary oil.

Another embodiment comprises cyclosporin A, Tego SMO 80 V®, and Rosemary oil.

Another embodiment comprises cyclosporin A, Tego SMO V®, and Rosemary oil.

Another embodiment comprises cyclosporin A, Tego SMS®, and Rosemary oil.

Another embodiment comprises cyclosporin A, Tego STO V®, and Rosemary oil.

Another embodiment comprises cyclosporin A, Tween 21®, and Rosemary oil.

Another embodiment comprises cyclosporin A, Tween 40®, and Rosemary oil.

Another embodiment comprises cyclosporin A, Tween 60®, and Rosemary oil.

Another embodiment comprises cyclosporin A, Tween 60 K®, and Rosemary oil.

Another embodiment comprises cyclosporin A, Tween 61®, and Rosemary oil.

Another embodiment comprises cyclosporin A, Tween 65®, and Rosemary oil.

Another embodiment comprises cyclosporin A, Tween 80®, and Rosemary oil.

Another embodiment comprises cyclosporin A, Tween 80 K®, and Rosemary oil.

Another embodiment comprises cyclosporin A, Tween 81®, and Rosemary oil.

Another embodiment comprises cyclosporin A, Tween 85®, and Rosemary oil.

Another embodiment comprises cyclosporin A, a Sucrose or Glucose Ester, or Derivative thereof, and Rosemary oil.

Another embodiment comprises cyclosporin A, DeSulf GOS-P-60WCG®, and Rosemary oil.

Another embodiment comprises cyclosporin A, Glucam E-20 Distearate®, and Rosemary oil.

Another embodiment comprises cyclosporin A, Glucamate DOE-120®, and Rosemary oil.

Another embodiment comprises cyclosporin A, Glucamate SSE-20®, and Rosemary oil.

Another embodiment comprises cyclosporin A, Glucate DO®, and Rosemary oil.

Another embodiment comprises cyclosporin A, Glucate SS®, and Rosemary oil.

Another embodiment comprises cyclosporin A, Glucopon 425 UP®, and Rosemary oil.

Another embodiment comprises cyclosporin A, Isolan IS®, and Rosemary oil.

Another embodiment comprises cyclosporin A, Mazon 40®, and Rosemary oil.

Another embodiment comprises cyclosporin A, Montanov 82®, and Rosemary oil.

Another embodiment comprises cyclosporin A, Montanov 202®, and Rosemary oil.

Another embodiment comprises cyclosporin A, Montanov S®, and Rosemary oil.

Another embodiment comprises cyclosporin A, Rheozan®, and Rosemary oil.

Another embodiment comprises cyclosporin A, Simulsol AS 48®, and Rosemary oil.

Another embodiment comprises cyclosporin A, Simulsol SL 4®, and Rosemary oil.

Another embodiment comprises cyclosporin A, Simulsol SL 10®, and Rosemary oil.

Another embodiment comprises cyclosporin A, Simulsol SL 11W®, and Rosemary oil.

Another embodiment comprises cyclosporin A, Simulsol SL 55®, and Rosemary oil.

Another embodiment comprises cyclosporin A, Suga Nate 100 and 160®, and Rosemary oil.

Another embodiment comprises cyclosporin A, Tego Care 450®, and Rosemary oil.

Another embodiment comprises cyclosporin A, Tego Care CG 90®, and Rosemary oil.

Another embodiment comprises cyclosporin A, Tego Care PS®, and Rosemary oil.

Another embodiment comprises cyclosporin A, Tegosoft PSE 141 G®, and Rosemary oil.

Another embodiment comprises cyclosporin A, Tegotens G 826®, and Rosemary oil.

Another embodiment comprises cyclosporin A, Triton BG-10 (70%)®, and Rosemary oil.

Another embodiment comprises cyclosporin A, Triton CG-110 (60%)®, and Rosemary oil.

Another embodiment comprises cyclosporin A, Wickenol 545®, and Rosemary oil.

Another embodiment comprises cyclosporin A, an alcohol, and Peanut oil.

Another embodiment comprises cyclosporin A, Diglycerol®, and Peanut oil.

Another embodiment comprises cyclosporin A, Hetoxide GT-80®, and Peanut oil.

Another embodiment comprises cyclosporin A, Lexemul BEO®, and Peanut oil.

Another embodiment comprises cyclosporin A, Polyglycerol-3®, and Peanut oil.

Another embodiment comprises cyclosporin A, Redicote E Series®, and Peanut oil.

Another embodiment comprises cyclosporin A, Simulsol OX 1005L®, and Peanut oil.

Another embodiment comprises cyclosporin A, Stanfax 567®, and Peanut oil.

Another embodiment comprises cyclosporin A, TA-1618®, and Peanut oil.

Another embodiment comprises cyclosporin A, Witconol H-31A®, and Peanut oil.

Another embodiment comprises cyclosporin A, an amine oxide, and Peanut oil.

Another embodiment comprises cyclosporin A, AO-405®, and Peanut oil.

Another embodiment comprises cyclosporin A, AO-455®, and Peanut oil.

Another embodiment comprises cyclosporin A, AO 728 Special®, and Peanut oil.

Another embodiment comprises cyclosporin A, Barlox 12®, and Peanut oil.

Another embodiment comprises cyclosporin A, Barlox 14®, and Peanut oil.

Another embodiment comprises cyclosporin A, Burcoxide Lo®, and Peanut oil.

Another embodiment comprises cyclosporin A, Caloxamine LO®, and Peanut oil.

Another embodiment comprises cyclosporin A, Chemoxide CAW®, and Peanut oil.

Another embodiment comprises cyclosporin A, Chemoxide LM-30®, and Peanut oil.

Another embodiment comprises cyclosporin A, Chemoxide LO®, and Peanut oil.

Another embodiment comprises cyclosporin A, Chemoxide MO®, and Peanut oil.

Another embodiment comprises cyclosporin A, Colalux CAO-35®, and Peanut oil.

Another embodiment comprises cyclosporin A, Colalux LO®, and Peanut oil.

Another embodiment comprises cyclosporin A, DeMox CAPO®, and Peanut oil.

Another embodiment comprises cyclosporin A, DeMox CSG-30®, and Peanut oil.

Another embodiment comprises cyclosporin A, DeMox LAO®, and Peanut oil.

Another embodiment comprises cyclosporin A, Emcol L®, and Peanut oil.

Another embodiment comprises cyclosporin A, Empigen OB®, and Peanut oil.

Another embodiment comprises cyclosporin A, Empigen OS/A®, and Peanut oil.

Another embodiment comprises cyclosporin A, Foamox CDO®, and Peanut oil.

Another embodiment comprises cyclosporin A, Foamox DMM®, and Peanut oil.

Another embodiment comprises cyclosporin A, Foamox DMS®, and Peanut oil.

Another embodiment comprises cyclosporin A, Genaminox KC®, and Peanut oil.

Another embodiment comprises cyclosporin A, Genaminox LA®, and Peanut oil.

Another embodiment comprises cyclosporin A, Hartofoam SAO®, and Peanut oil.

Another embodiment comprises cyclosporin A, Hartox DMCD®, and Peanut oil.

Another embodiment comprises cyclosporin A, Lipowax DAT®, and Peanut oil.

Another embodiment comprises cyclosporin A, Lipowax PB Pastilles®, and Peanut oil.

Another embodiment comprises cyclosporin A, Mackamine C8®, and Peanut oil.

Another embodiment comprises cyclosporin A, Mackamine C10®, and Peanut oil.

Another embodiment comprises cyclosporin A, Mackamine C14®, and Peanut oil.

Another embodiment comprises cyclosporin A, Mackamine CAO®, and Peanut oil.

Another embodiment comprises cyclosporin A, Mackamine CO®, and Peanut oil.

Another embodiment comprises cyclosporin A, Mackamine LO®, and Peanut oil.

Another embodiment comprises cyclosporin A, Mackamine O2®, and Peanut oil.

Another embodiment comprises cyclosporin A, Mackamine SAO®, and Peanut oil.

Another embodiment comprises cyclosporin A, Mackamine SO®, and Peanut oil.

Another embodiment comprises cyclosporin A, Mazox KCAO®, and Peanut oil.

Another embodiment comprises cyclosporin A, Monalac MO®, and Peanut oil.

Another embodiment comprises cyclosporin A, Norfox LDA®, and Peanut oil.

Another embodiment comprises cyclosporin A, Rhodamox LO®, and Peanut oil.

Another embodiment comprises cyclosporin A, Schercamox C-AA®, and Peanut oil.

Another embodiment comprises cyclosporin A, Schercamox DMA®, and Peanut oil.

Another embodiment comprises cyclosporin A, Schercamox DML®, and Peanut oil.

Another embodiment comprises cyclosporin A, Schercamox DMM®, and Peanut oil.

Another embodiment comprises cyclosporin A, Schercamox DMS®, and Peanut oil.

Another embodiment comprises cyclosporin A, Tegotens DO®, and Peanut oil.

Another embodiment comprises cyclosporin A, Tomah AO-14-2®, and Peanut oil.

Another embodiment comprises cyclosporin A, Triaminox CDO®, and Peanut oil.

Another embodiment comprises cyclosporin A, a block polymer, and Peanut oil.

Another embodiment comprises cyclosporin A, AL 2070®, and Peanut oil.

Another embodiment comprises cyclosporin A, Antarox 17-R-2®, and Peanut oil.

Another embodiment comprises cyclosporin A, Antarox 25-R-2®, and Peanut oil.

Another embodiment comprises cyclosporin A, Antarox 31,-R-1®, and Peanut oil.

Another embodiment comprises cyclosporin A, Antarox P-84®, and Peanut oil.

Another embodiment comprises cyclosporin A, Antarox P-104/H®, and Peanut oil.

Another embodiment comprises cyclosporin A, Arnox BP-Series®, and Peanut oil.

Another embodiment comprises cyclosporin A, Chemonic 435®, and Peanut oil.

Another embodiment comprises cyclosporin A, Chemonic D-25®, and Peanut oil.

Another embodiment comprises cyclosporin A, Chemonic PL Series®, and Peanut oil.

Another embodiment comprises cyclosporin A, Ethox L-121®, and Peanut oil.

Another embodiment comprises cyclosporin A, Ethox L-122®, and Peanut oil.

Another embodiment comprises cyclosporin A, Genapol PF-10®, and Peanut oil.

Another embodiment comprises cyclosporin A, Genapol PF-20®, and Peanut oil.

Another embodiment comprises cyclosporin A, Genapol PF-40A®, and Peanut oil.

Another embodiment comprises cyclosporin A, Nortox 2-LF®, and Peanut oil.

Another embodiment comprises cyclosporin A, Simulsol NW 342®, and Peanut oil.

Another embodiment comprises cyclosporin A, T-Det BP-1®, and Peanut oil.

Another embodiment comprises cyclosporin A, T-Det XD®), and Peanut oil.

Another embodiment comprises cyclosporin A, T-Det XH®, and Peanut oil.

Another embodiment comprises cyclosporin A, Triton CF-32®, and Peanut oil.

Another embodiment comprises cyclosporin A, Witconol 171®, and Peanut oil.

Another embodiment comprises cyclosporin A, Witconol 324®, and Peanut oil.

Another embodiment comprises cyclosporin A, Witconol 324D®, and Peanut oil.

Another embodiment comprises cyclosporin A, Witconol PD-2000®, and Peanut oil.

Another embodiment comprises cyclosporin A, a Carboxylated Alcohol or Alkylphenol Ethoxylate, and Peanut oil.

Another embodiment comprises cyclosporin A, Emcol CN-6®, and Peanut oil.

Another embodiment comprises cyclosporin A, Ethcarb®, and Peanut oil.

Another embodiment comprises cyclosporin A, Gemtex WNT-Conc®, and Peanut oil.

Another embodiment comprises cyclosporin A, Incrodet TD7-C®, and Peanut oil.

Another embodiment comprises cyclosporin A, Marlinat CM 105/80®, and Peanut oil.

Another embodiment comprises cyclosporin A, Marlowet 1072®, and Peanut oil.

Another embodiment comprises cyclosporin A, Marlowet 4530®, and Peanut oil.

Another embodiment comprises cyclosporin A, Marlowet 4530 LF®, and Peanut oil.

Another embodiment comprises cyclosporin A, Marlowet 4534®, and Peanut oil.

Another embodiment comprises cyclosporin A, Marlowet 4538®, and Peanut oil.

Another embodiment comprises cyclosporin A, Marlowet 4539®, and Peanut oil.

Another embodiment comprises cyclosporin A, Marlowet 4539 LF®, and Peanut oil.

Another embodiment comprises cyclosporin A, Marlowet 4541®, and Peanut oil.

Another embodiment comprises cyclosporin A, Miranate LEC-80®, and Peanut oil.

Another embodiment comprises cyclosporin A, Sandopan B®, and Peanut oil.

Another embodiment comprises cyclosporin A, Sandopan B Modified®, and Peanut oil.

Another embodiment comprises cyclosporin A, Sandopan LS-24 Gel®, and Peanut oil.

Another embodiment comprises cyclosporin A, Surfine T-A®, and Peanut oil.

Another embodiment comprises cyclosporin A, a Carboxylic Acid/Fatty Acid, and Peanut oil.

Another embodiment comprises cyclosporin A, Colaterge RAM®, and Peanut oil.

Another embodiment comprises cyclosporin A, Colatrope INC®, and Peanut oil.

Another embodiment comprises cyclosporin A, Crodacid B®, and Peanut oil.

Another embodiment comprises cyclosporin A, DeTrope CA-100®, and Peanut oil.

Another embodiment comprises cyclosporin A, Latol MTO®, and-Peanut oil.

Another embodiment comprises cyclosporin A, Lumulse CC-33 K®, and Peanut oil.

Another embodiment comprises cyclosporin A, Mulls 2218®, and Peanut oil.

Another embodiment comprises cyclosporin A, OL-600®, and Peanut oil.

Another embodiment comprises cyclosporin A, OL-800®, and Peanut oil.

Another embodiment comprises cyclosporin A, R-910®, and Peanut oil.

Another embodiment comprises cyclosporin A, S-210®, and Peanut oil.

Another embodiment comprises cyclosporin A, Sandopan DTC Acid®, and Peanut oil.

Another embodiment comprises cyclosporin A, Sandopan LS 24 N®, and Peanut oil.

Another embodiment comprises cyclosporin A, Sandopan MA-18®, and Peanut oil.

Another embodiment comprises cyclosporin A, an Ethoxylated Alcohol, and Peanut oil.

Another embodiment comprises cyclosporin A, Adsee 799®, and Peanut oil.

Another embodiment comprises cyclosporin A, Adsee 799®, and Peanut oil.

Another embodiment comprises cyclosporin A, Alfonic 610-3.5®, and Peanut oil.

Another embodiment comprises cyclosporin A, Alfonic 810-2®, and Peanut oil.

Another embodiment comprises cyclosporin A, Alfonic 810-6®, and Peanut oil.

Another embodiment comprises cyclosporin A, Alfonic 1012-3®, and Peanut oil.

Another embodiment comprises cyclosporin A, Alfonic 1012-5®, and Peanut oil.

Another embodiment comprises cyclosporin A, Alfonic 1216CO-1.5®, and Peanut oil.

Another embodiment comprises cyclosporin A, Alfonic 1216CO-7®, and Peanut oil.

Another embodiment comprises cyclosporin A, Alfonic 1412-3®, and Peanut oil.

Another embodiment comprises cyclosporin A, Alfonic 1412-7®, and Peanut oil.

Another embodiment comprises cyclosporin A, Arlasolve 200®, and Peanut oil.

Another embodiment comprises cyclosporin A, Arlasolve 200 Liquid®, and Peanut oil.

Another embodiment comprises cyclosporin A, Armix 180-C®, and Peanut oil.

Another embodiment comprises cyclosporin A, Armix 183®, and Peanut oil.

Another embodiment comprises cyclosporin A, Armul 2404®, and Peanut oil.

Another embodiment comprises cyclosporin A, Armul 2404®, and Peanut oil.

Another embodiment comprises cyclosporin A, Atlas EMJ-C®, and Peanut oil.

Another embodiment comprises cyclosporin A, Atlas G-2109®, and Peanut oil.

Another embodiment comprises cyclosporin A, Atlas G-3886®, and Peanut oil.

Another embodiment comprises cyclosporin A, Atlas G-3890®, and Peanut oil.

Another embodiment comprises cyclosporin A, Bio Soft E-200®, and Peanut oil.

Another embodiment comprises cyclosporin A, Bio Soft E-300®, and Peanut oil.

Another embodiment comprises cyclosporin A, Bio Soft E-400®, and Peanut oil.

Another embodiment comprises cyclosporin A, Bio Soft EN 600®, and Peanut oil.

Another embodiment comprises cyclosporin A, Bio Soft TD-400®, and Peanut oil.

Another embodiment comprises cyclosporin A, Bio Soft TD-630®, and Peanut oil.

Another embodiment comprises cyclosporin A, Brij 30®, and Peanut oil.

Another embodiment comprises cyclosporin A, Brij 52®, and Peanut oil.

Another embodiment comprises cyclosporin A, Brij 56®, and Peanut oil.

Another embodiment comprises cyclosporin A, Brij 58®, and Peanut oil.

Another embodiment comprises cyclosporin A, Brij 72®, and Peanut oil.

Another embodiment comprises cyclosporin A, Brij 76®, and Peanut oil.

Another embodiment comprises cyclosporin A, Brij 78®, and Peanut oil.

Another embodiment comprises cyclosporin A, Brij 93®, and Peanut oil.

Another embodiment comprises cyclosporin A, Brij 97®, and Peanut oil.

Another embodiment comprises cyclosporin A, Brij 98®, and Peanut oil.

Another embodiment comprises cyclosporin A, Brij 700®, and Peanut oil.

Another embodiment comprises cyclosporin A, Brij 700 S®, and Peanut oil.

Another embodiment comprises cyclosporin A, Brij 721®, and Peanut oil.

Another embodiment comprises cyclosporin A, Brij 721 S®, and Peanut oil.

Another embodiment comprises cyclosporin A, Burcoterge CDG®, and Peanut oil.

Another embodiment comprises cyclosporin A, Canasol AT 600®, and Peanut oil.

Another embodiment comprises cyclosporin A, Canasol AT 800®, and Peanut oil.

Another embodiment comprises cyclosporin A, Canasol AT 1200®, and Peanut oil.

Another embodiment comprises cyclosporin A, Canasol BJ 35®, and Peanut oil.

Another embodiment comprises cyclosporin A, Canasol BJ 36®, and Peanut oil.

Another embodiment comprises cyclosporin A, Canasol BJ 52®, and Peanut oil.

Another embodiment comprises cyclosporin A, Canasol BJ 58®, and Peanut oil.

Another embodiment comprises cyclosporin A, Canasol BJ 72®, and Peanut oil.

Another embodiment comprises cyclosporin A, Canasol BJ 78®, and Peanut oil.

Another embodiment comprises cyclosporin A, Canasol BJ 98®, and Peanut oil.

Another embodiment comprises cyclosporin A, Canasol BJ 307®, and Peanut oil.

Another embodiment comprises cyclosporin A, Cenak 1400®, and Peanut oil.

Another embodiment comprises cyclosporin A, Cetomacrogol 1000 BP®, and Peanut oil.

Another embodiment comprises cyclosporin A, Chemonic C-2®, and Peanut oil.

Another embodiment comprises cyclosporin A, Chemonic C-10®, and Peanut oil.

Another embodiment comprises cyclosporin A, Chemonic C-20®, and Peanut oil.

Another embodiment comprises cyclosporin A, Chemonic CT-12®, and Peanut oil.

Another embodiment comprises cyclosporin A, Chemonic CT-20®, and Peanut oil.

Another embodiment comprises cyclosporin A, Chemonic CT-30®, and Peanut oil.

Another embodiment comprises cyclosporin A, Chemonic CT-55®, and Peanut oil.

Another embodiment comprises cyclosporin A, Chemonic G-7®, and Peanut oil.

Another embodiment comprises cyclosporin A, Chemonic G-26®, and Peanut oil.

Another embodiment comprises cyclosporin A, Chemonic L-4®, and Peanut oil.

Another embodiment comprises cyclosporin A, Chemonic L-7®, and Peanut oil.

Another embodiment comprises cyclosporin A, Chemonic L-12®, and Peanut oil.

Another embodiment comprises cyclosporin A, Chemonic L-23®, and Peanut oil.

Another embodiment comprises cyclosporin A, Chemonic O-2®, and Peanut oil.

Another embodiment comprises cyclosporin A, Chemonic O-5®, and Peanut oil.

Another embodiment comprises cyclosporin A, Chemonic O-10®, and Peanut oil.

Another embodiment comprises cyclosporin A, Chemonic O-20®, and Peanut oil.

Another embodiment comprises cyclosporin A, Chemonic S-2®, and Peanut oil.

Another embodiment comprises cyclosporin A, Chemonic S-10®, and Peanut oil.

Another embodiment comprises cyclosporin A, Chemonic S-20®, and Peanut oil.

Another embodiment comprises cyclosporin A, Colamulse FE®, and Peanut oil.

Another embodiment comprises cyclosporin A, Cremophor A 20®, and Peanut oil.

Another embodiment comprises cyclosporin A, Cremophor SA 2®, and Peanut oil.

Another embodiment comprises cyclosporin A, Dehydol 100®, and Peanut oil.

Another embodiment comprises cyclosporin A, Dehydol O-4®, and Peanut oil.

Another embodiment comprises cyclosporin A, Delonic C-18®, and Peanut oil.

Another embodiment comprises cyclosporin A, DeSonic 6T®, and Peanut oil.

Another embodiment comprises cyclosporin A, DeSonic 9D®, and Peanut oil.

Another embodiment comprises cyclosporin A, DeSonic 9T®, and Peanut oil.

Another embodiment comprises cyclosporin A, DeSonic 12D®, and Peanut oil.

Another embodiment comprises cyclosporin A, DeSonic 12T®, and Peanut oil.

Another embodiment comprises cyclosporin A, DeSonic 15T®, and Peanut oil.

Another embodiment comprises cyclosporin A, DeSonic TDA-9®, and Peanut oil.

Another embodiment comprises cyclosporin A, DeThox GLG-7®, and Peanut oil.

Another embodiment comprises cyclosporin A, DeThox GLG-26®, and Peanut oil.

Another embodiment comprises cyclosporin A, DeThox LA-4®, and Peanut oil.

Another embodiment comprises cyclosporin A, DeThox LA-23®, and Peanut oil.

Another embodiment comprises cyclosporin A, DeThox SA-80®, and Peanut oil.

Another embodiment comprises cyclosporin A, Disponil O5®, and Peanut oil.

Another embodiment comprises cyclosporin A, Eccoterge EO-41 B®, and Peanut oil.

Another embodiment comprises cyclosporin A, Empilan KA2.5/90®, and Peanut oil.

Another embodiment comprises cyclosporin A, Empilan KA5/90®, and Peanut oil.

Another embodiment comprises cyclosporin A, Empilan KM-20®, and Peanut oil.

Another embodiment comprises cyclosporin A, Empilan KM-50®, and Peanut oil.

Another embodiment comprises cyclosporin A, Empilan L-23®, and Peanut oil.

Another embodiment comprises cyclosporin A, Ethylan 25-3®, and Peanut oil.

Another embodiment comprises cyclosporin A, Ethylan 1204®, and Peanut oil.

Another embodiment comprises cyclosporin A, Ethylan DA-4®, and Peanut oil.

Another embodiment comprises cyclosporin A, Ethylan LA-230®, and Peanut oil.

Another embodiment comprises cyclosporin A, Ethylan SN®, and Peanut oil.

Another embodiment comprises cyclosporin A, Ethylan TD-60®, and Peanut oil.

Another embodiment comprises cyclosporin A, Ethylan TD-100®, and Peanut oil.

Another embodiment comprises cyclosporin A, Ethylan TD-1407®, and Peanut oil.

Another embodiment comprises cyclosporin A, Eumulgin B1®, and Peanut oil.

Another embodiment comprises cyclosporin A, Eumulgin B2®, and Peanut oil.

Another embodiment comprises cyclosporin A, Eumulgin B3®, and Peanut oil.

Another embodiment comprises cyclosporin A, Eumulgin O-10®, and Peanut oil.

Another embodiment comprises cyclosporin A, Flo Mo 80/20®, and Peanut oil.

Another embodiment comprises cyclosporin A, Flo Mo Low Foam®, and Peanut oil.

Another embodiment comprises cyclosporin A, Forlan C-24®, and Peanut oil.

Another embodiment comprises cyclosporin A, Genapol 1454®, and Peanut oil.

Another embodiment comprises cyclosporin A, Genapol BA-020®, and Peanut oil.

Another embodiment comprises cyclosporin A, Genapol BA-040®, and Peanut oil.

Another embodiment comprises cyclosporin A, Genapol C-100®, and Peanut oil.

Another embodiment comprises cyclosporin A, Genapol DA 060®, and Peanut oil.

Another embodiment comprises cyclosporin A, Genapol HS 020®, and Peanut oil.

Another embodiment comprises cyclosporin A, Genapol HS 200®, and Peanut oil.

Another embodiment comprises cyclosporin A, Genapol ID-040®, and Peanut oil.

Another embodiment comprises cyclosporin A, Genapol ID-060®, and Peanut oil.

Another embodiment comprises cyclosporin A, Genapol ID-090®, and Peanut oil.

Another embodiment comprises cyclosporin A, Genapol LA 010®, and Peanut oil.

Another embodiment comprises cyclosporin A, Genapol LA 020®, and Peanut oil.

Another embodiment comprises cyclosporin A, Genapol LA 030®, and Peanut oil.

Another embodiment comprises cyclosporin A, Genapol LA 040®, and Peanut oil.

Another embodiment comprises cyclosporin A, Genapol LA 050®, and Peanut oil.

Another embodiment comprises cyclosporin A, Genapol LA 060®, and Peanut oil.

Another embodiment comprises cyclosporin A, Genapol LA 070®, and Peanut oil.

Another embodiment comprises cyclosporin A, Genapol LA 070S®, and Peanut oil.

Another embodiment comprises cyclosporin A, Genapol LA 230®, and Peanut oil.

Another embodiment comprises cyclosporin A, Genapol O 020®, and Peanut oil.

Another embodiment comprises cyclosporin A, Genapol O 050®, and Peanut oil.

Another embodiment comprises cyclosporin A, Genapol O 100®, and Peanut oil.

Another embodiment comprises cyclosporin A, Genapol O 200®, and Peanut oil.

Another embodiment comprises cyclosporin A, Genapol SA 030®, and Peanut oil.

Another embodiment comprises cyclosporin A, Genapol SA 120®, and Peanut oil.

Another embodiment comprises cyclosporin A, Genapol T-020®, and Peanut oil.

Another embodiment comprises cyclosporin A, Genapol UD-030®, and Peanut oil.

Another embodiment comprises cyclosporin A, Genapol UD-050®, and Peanut oil.

Another embodiment comprises cyclosporin A, Genapol UD-070®, and Peanut oil.

Another embodiment comprises cyclosporin A, Genapol UD-079®, and Peanut oil.

Another embodiment comprises cyclosporin A, Genapol UD-080®, and Peanut oil.

Another embodiment comprises cyclosporin A, Genapol UD-110®, and Peanut oil.

Another embodiment comprises cyclosporin A, Genapol X 030®, and Peanut oil.

Another embodiment comprises cyclosporin A, Genapol X 050®, and Peanut oil.

Another embodiment comprises cyclosporin A, Genapol X 060®, and Peanut oil.

Another embodiment comprises cyclosporin A, Genapol X 070®, and Peanut oil.

Another embodiment comprises cyclosporin A, Genapol X 080®, and Peanut oil.

Another embodiment comprises cyclosporin A, Genapol X 100®, and Peanut oil.

Another embodiment comprises cyclosporin A, Genapol X159®, and Peanut oil.

Another embodiment comprises cyclosporin A, Generol 122 E5®, and Peanut oil.

Another embodiment comprises cyclosporin A, Generol 122 E25®, and Peanut oil.

Another embodiment comprises cyclosporin A, Hostacerin T-3®, and Peanut oil.

Another embodiment comprises cyclosporin A, Iconol LF 110®, and Peanut oil.

Another embodiment comprises cyclosporin A, Incropol CS-20®, and Peanut oil.

Another embodiment comprises cyclosporin A, Lexemul CS-20®, and Peanut oil.

Another embodiment comprises cyclosporin A, Liponic EG-1®, and Peanut oil.

Another embodiment comprises cyclosporin A, Lipowax D®, and Peanut oil.

Another embodiment comprises cyclosporin A, Lipowax G®, and Peanut oil.

Another embodiment comprises cyclosporin A, Lipowax NI®, and Peanut oil.

Another embodiment comprises cyclosporin A, Lipowax P®, and Peanut oil.

Another embodiment comprises cyclosporin A, Lipowax P-31®, and Peanut oil.

Another embodiment comprises cyclosporin A, Lipowax PR®, and Peanut oil.

Another embodiment comprises cyclosporin A, Lumulse CS-20®, and Peanut oil.

Another embodiment comprises cyclosporin A, Macol CSA-20®, and Peanut oil.

Another embodiment comprises cyclosporin A, Marlox B 24/50®, and Peanut oil.

Another embodiment comprises cyclosporin A, Mazawet 77®, and Peanut oil.

Another embodiment comprises cyclosporin A, No nox 1713®, and Peanut oil.

Another embodiment comprises cyclosporin A, Norfox 2579®, and Peanut oil.

Another embodiment comprises cyclosporin A, Norfox Lo Foam®, and Peanut oil.

Another embodiment comprises cyclosporin A, Promulgen D®, and Peanut oil.

Another embodiment comprises cyclosporin A, Promulgen G®, and Peanut oil.

Another embodiment comprises cyclosporin A, Renex 30®, and Peanut oil.

Another embodiment comprises cyclosporin A, Renex 36®, and Peanut oil.

Another embodiment comprises cyclosporin A, Rhodasurf A 24®, and Peanut oil.

Another embodiment comprises cyclosporin A, Rhodasur AAE-10®, and Peanut oil.

Another embodiment comprises cyclosporin A, Rhodasurf BEH-25®, and Peanut oil.

Another embodiment comprises cyclosporin A, Rhodasurf BEH-40®, and Peanut oil.

Another embodiment comprises cyclosporin A, Rhodasurf DA 530®, and Peanut oil.

Another embodiment comprises cyclosporin A, Rhodasurf DA 630®, and Peanut oil.

Another embodiment comprises cyclosporin A, Rhodasurf DA 639®, and Peanut oil.

Another embodiment comprises cyclosporin A, Rhodasurf LAN-23®, and Peanut oil.

Another embodiment comprises cyclosporin A, Rhodasurf ON-870®, and Peanut oil.

Another embodiment comprises cyclosporin A, Rhodasurf ON-877®, and Peanut oil.

Another embodiment comprises cyclosporin A, Rhodasurf TB-970 FLK®, and Peanut oil.

Another embodiment comprises cyclosporin A, Ritacet-20®, and Peanut oil.

Another embodiment comprises cyclosporin A, Ritachol 1000®, and Peanut oil.

Another embodiment comprises cyclosporin A, Ritachol 2000®, and Peanut oil.

Another embodiment comprises cyclosporin A, Ritachol 5000®, and Peanut oil.

Another embodiment comprises cyclosporin A, Ritox 35®, and Peanut oil.

Another embodiment comprises cyclosporin A, Surfonic DA-4®, and Peanut oil.

Another embodiment comprises cyclosporin A, Surfonic DA-6®, and Peanut oil.

Another embodiment comprises cyclosporin A, Surfonic L46-7®, and Peanut oil.

Another embodiment comprises cyclosporin A, Surfonic POA®, and Peanut oil.

Another embodiment comprises cyclosporin A, Synthrapol KB®, and Peanut oil.

Another embodiment comprises cyclosporin A, Teginacid®, and Peanut oil.

Another embodiment comprises cyclosporin A, Teginacid C®, and Peanut oil.

Another embodiment comprises cyclosporin A, Tegotens EC 11®, and Peanut oil.

Another embodiment comprises cyclosporin A, Tinegal NA®, and Peanut oil.

Another embodiment comprises cyclosporin A, Tomadol 400®, and Peanut oil.

Another embodiment comprises cyclosporin A, Tomadol 600®, and Peanut oil.

Another embodiment comprises cyclosporin A, Tomadol 900®, and Peanut oil.

Another embodiment comprises cyclosporin A, Uniperol O®, and Peanut oil.

Another embodiment comprises cyclosporin A, Witconol SN Series®, and Peanut oil.

Another embodiment comprises cyclosporin A, an Ethoxylated Alkylphenol, and Peanut oil.

Another embodiment comprises cyclosporin A, Antarox LF-222®, and Peanut oil.

Another embodiment comprises cyclosporin A, Atlox 775®, and Peanut oil.

Another embodiment comprises cyclosporin A, Caloxylate N-9®, and Peanut oil.

Another embodiment comprises cyclosporin A, Canasol NF-1000®, and Peanut oil.

Another embodiment comprises cyclosporin A, Canasol NF-3000®, and Peanut oil.

Another embodiment comprises cyclosporin A, Canasol NF-3070®, and Peanut oil.

Another embodiment comprises cyclosporin A, Canasol OF 1670®, and Peanut oil.

Another embodiment comprises cyclosporin A, Canasol OF 2570®, and Peanut oil.

Another embodiment comprises cyclosporin A, Canasol OF 4070®, and Peanut oil.

Another embodiment comprises cyclosporin A, Chemax DNP-8®, and Peanut oil.

Another embodiment comprises cyclosporin A, Chemax DNP-18®, and Peanut oil.

Another embodiment comprises cyclosporin A, Chemax DNP-150/50®, and Peanut oil.

Another embodiment comprises cyclosporin A, DeSonic 1.5N®, and Peanut oil.

Another embodiment comprises cyclosporin A, DeSonic 4N®, and Peanut oil.

Another embodiment comprises cyclosporin A, DeSonic 5N®, and Peanut oil.

Another embodiment comprises cyclosporin A, DeSonic 6D®, and Peanut oil.

Another embodiment comprises cyclosporin A, DeSonic 6N®, and Peanut oil.

Another embodiment comprises cyclosporin A, DeSonic 7N®, and Peanut oil.

Another embodiment comprises cyclosporin A, DeSonic 9N®, and Peanut oil.

Another embodiment comprises cyclosporin A, DeSonic 10D®, and Peanut oil.

Another embodiment comprises cyclosporin A, DeSonic 11N®, and Peanut oil.

Another embodiment comprises cyclosporin A, DeSonic 12N®, and Peanut oil.

Another embodiment comprises cyclosporin A, DeSonic 15N®, and Peanut oil.

Another embodiment comprises cyclosporin A, DeSonic 20N®, and Peanut oil.

Another embodiment comprises cyclosporin A, Eccoscour RC®, and Peanut oil.

Another embodiment comprises cyclosporin A, Eccoterge EO-100®, and Peanut oil.

Another embodiment comprises cyclosporin A, Emulsifier 632/90%®, and Peanut oil.

Another embodiment comprises cyclosporin A, Geronol AG-821®, and Peanut oil.

Another embodiment comprises cyclosporin A, Gradonic N-95®, and Peanut oil.

Another embodiment comprises cyclosporin A, Hetoxide NP-4®, and Peanut oil.

Another embodiment comprises cyclosporin A, Hetoxide NP-30®, and Peanut oil.

Another embodiment comprises cyclosporin A, Hostapal N-100®, and Peanut oil.

Another embodiment comprises cyclosporin A, Hostapal N-110®, and Peanut oil.

Another embodiment comprises cyclosporin A, Igepal CTA-639W®, and Peanut oil.

Another embodiment comprises cyclosporin A, Igepal DAP-9®, and Peanut oil.

Another embodiment comprises cyclosporin A, Igepal OD-410®, and Peanut oil. tO

Another embodiment comprises cyclosporin A, Igepal SS-837®, and Peanut oil.

Another embodiment comprises cyclosporin A, Lipocol NP-9 USP®, and Peanut oil.

Another embodiment comprises cyclosporin A, Macol DNP-10®, and Peanut oil.

Another embodiment comprises cyclosporin A, Marlophen NP 5®, and Peanut oil.

Another embodiment comprises cyclosporin A, Marlophen P 1®, and Peanut oil.

Another embodiment comprises cyclosporin A, Surfonic NB®, and Peanut oil.

Another embodiment comprises cyclosporin A, Surfonic OPB-307®, and Peanut oil.

Another embodiment comprises cyclosporin A, Surfonic OPB-407®, and Peanut oil.

Another embodiment comprises cyclosporin A, Syn Fac 334®, and Peanut oil.

Another embodiment comprises cyclosporin A, Syn Fac 8216®, and Peanut oil.

Another embodiment comprises cyclosporin A, Triton N-57®, and Peanut oil.

Another embodiment comprises cyclosporin A, Trycol 6956®, and Peanut oil.

Another embodiment comprises cyclosporin A, Trycol 6961®, and Peanut oil.

Another embodiment comprises cyclosporin A, Trycol 6964®, and Peanut oil.

Another embodiment comprises cyclosporin A, Trycol 6969®, and Peanut-oil.

Another embodiment comprises cyclosporin A, Trycol 6974®, and Peanut oil.

Another embodiment comprises cyclosporin A, Witbreak DRB-127®, and Peanut oil.

Another embodiment comprises cyclosporin A, Witbreak DRB-127®, and Peanut oil.

Another embodiment comprises cyclosporin A, Witconol NP Series®, and Peanut oil.

Another embodiment comprises cyclosporin A, an Ethoxylated Aryl Phenol, and Peanut oil.

Another embodiment comprises cyclosporin A, Soprophor BSU®, and Peanut oil.

Another embodiment comprises cyclosporin A, Soprophor CY/8®, and Peanut oil.

Another embodiment comprises cyclosporin A, Soprophor S/25®, and Peanut oil.

Another embodiment comprises cyclosporin A, Witconol NIO®, and Peanut oil.

Another embodiment comprises cyclosporin A, Witconol NIW®, and Peanut oil.

Another embodiment comprises cyclosporin A, Witconol S-100®, and Peanut oil.

Another embodiment comprises cyclosporin A, an Ethoxylated Fatty Acid, and Peanut oil.

Another embodiment comprises cyclosporin A, Aldo PGHMS®, and Peanut oil.

Another embodiment comprises cyclosporin A, Alkamuls TO-15/HR®, and Peanut oil.

Another embodiment comprises cyclosporin A, Armotan AL-69-66®, and Peanut oil.

Another embodiment comprises cyclosporin A, Cerasynt 840®, and Peanut oil.

Another embodiment comprises cyclosporin A, Cerasynt 945®, and Peanut oil.

Another embodiment comprises cyclosporin A, Crystal Inhibitor No. 5®, and Peanut oil.

Another embodiment comprises cyclosporin A, DeThox Acid L-9®, and Peanut oil.

Another embodiment comprises cyclosporin A, DeThox Acid S-8®, and Peanut oil.

Another embodiment comprises cyclosporin A, Ethofat 242/25®, and Peanut oil.

Another embodiment comprises cyclosporin A, Hydropalat 65®, and Peanut oil.

Another embodiment comprises cyclosporin A, Lipo EGMS®, and Peanut oil.

Another embodiment comprises cyclosporin A, Lipopeg 2 DL®, and Peanut oil.

Another embodiment comprises cyclosporin A, Lipopeg 4 DL®, and Peanut oil.

Another embodiment comprises cyclosporin A, Lipopeg 4-L®, and Peanut oil.

Another embodiment comprises cyclosporin A, Lipopeg 39-S®, and Peanut oil.

Another embodiment comprises cyclosporin A, Lipopeg 4-S®, and Peanut oil.

Another embodiment comprises cyclosporin A, Lipopeg 10-S®, and Peanut oil.

Another embodiment comprises cyclosporin A, Lipopeg 100-S®, and Peanut oil.

Another embodiment comprises cyclosporin A, Lipopeg 6000 DS®, and Peanut oil.

Another embodiment comprises cyclosporin A, Lumulse 40-L®, and Peanut oil.

Another embodiment comprises cyclosporin A, Lumulse 40-S®, and Peanut oil.

Another embodiment comprises cyclosporin A, Lumulse 42-L®, and Peanut oil.

Another embodiment comprises cyclosporin A, Lumulse 42-S®, and Peanut oil.

Another embodiment comprises cyclosporin A, Lumulse 100-S®, and Peanut oil.

Another embodiment comprises cyclosporin A, Lumulse 602-S®, and Peanut oil.

Another embodiment comprises cyclosporin A, Magrabar PGE-20-0®, and Peanut oil.

Another embodiment comprises cyclosporin A, Magrabar PGE-20L®, and Peanut oil.

Another embodiment comprises cyclosporin A, Magrabar PGE-20T®, and Peanut oil.

Another embodiment comprises cyclosporin A, Magrabar PGE-22-0®, and Peanut oil.

Another embodiment comprises cyclosporin A, Magrabar PGE-22L®, and Peanut oil.

Another embodiment comprises cyclosporin A, Magrabar PGE-22T®, and Peanut oil.

Another embodiment comprises cyclosporin A, Magrabar PGE-40-0®, and Peanut oil.

Another embodiment comprises cyclosporin A, Magrabar PGE-40L®, and Peanut oil.

Another embodiment comprises cyclosporin A, Magrabar PGE-40T®, and Peanut oil.

Another embodiment comprises cyclosporin A, Magrabar PGE-42-0®, and Peanut oil.

Another embodiment comprises cyclosporin A, Magrabar PGE-42L®, and Peanut oil.

Another embodiment comprises cyclosporin A, Magrabar PGE-42T®, and Peanut oil.

Another embodiment comprises cyclosporin A, Magrabar PGE-60-0®, and Peanut oil.

Another embodiment comprises cyclosporin A, Magrabar PGE-60L®, and Peanut oil.

Another embodiment comprises cyclosporin A, Magrabar PGE-60T®, and Peanut oil.

Another embodiment comprises cyclosporin A, Magrabar PGE-62-0®, and Peanut oil.

Another embodiment comprises cyclosporin A, Magrabar PGE-62L®, and Peanut oil.

Another embodiment comprises cyclosporin A, Magrabar PGE-62T®, and Peanut oil.

Another embodiment comprises cyclosporin A, Mapeg S-40K®, and Peanut oil.

Another embodiment comprises cyclosporin A, Marlowet OTS®, and Peanut oil.

Another embodiment comprises cyclosporin A, Naturechem PGR®, and Peanut oil.

Another embodiment comprises cyclosporin A, PG No. 4®, and Peanut oil.

Another embodiment comprises cyclosporin A, Renex 20®, and Peanut oil.

Another embodiment comprises cyclosporin A, Ritox 52®, and Peanut oil.

Another embodiment comprises cyclosporin A, Ritox 53®, and Peanut oil.

Another embodiment comprises cyclosporin A, Ritox 59®, and Peanut oil.

Another embodiment comprises cyclosporin A, Surfax 8916/A®, and Peanut oil.

Another embodiment comprises cyclosporin A, Tego Acid S 40 P®, and Peanut oil.

Another embodiment comprises cyclosporin A, Tego Acid S 100 P®, and Peanut oil.

Another embodiment comprises cyclbsporin A, Tween 20®, and Peanut oil.

Another embodiment comprises cyclosporin A, Volpo 131®, and Peanut oil.

Another embodiment comprises cyclosporin A, an Ethoxylated Fatty Ester or Oil, and Peanut oil.

Another embodiment comprises cyclosporin A, Acconbn 6-C10®, and Peanut oil.

Another embodiment comprises cyclosporin A, Acconon CC-6®, and Peanut oil.

Another embodiment comprises cyclosporin A, Acconon CO-7®, and Peanut oil.

Another embodiment comprises cyclosporin A, Aldosperse 40/60 FG®, and Peanut oil.

Another embodiment comprises cyclosporin A, Aldosperse ML-23®, and Peanut oil.

Another embodiment comprises cyclosporin A, Aldosperse MS-20 FG®, and Peanut oil.

Another embodiment comprises cyclosporin A, Alkamuls EL-620®, and Peanut oil.

Another embodiment comprises cyclosporin A, Alkamuls EL-719®, and Peanut oil.

Another embodiment comprises cyclosporin A, Alkamuls EL-985®, and Peanut oil.

Another embodiment comprises cyclosporin A, Arlatone G®, and Peanut oil.

Another embodiment comprises cyclosporin A, Arlatone T®, and Peanut oil.

Another embodiment comprises cyclosporin A, Atlas G-1045A®, and Peanut oil.

Another embodiment comprises cyclosporin A, Atlas G-1086®, and Peanut oil.

Another embodiment comprises cyclosporin A, Atlas G-1087®, and Peanut oil.

Another embodiment comprises cyclosporin A, Atlas G-1089®, and Peanut oil.

Another embodiment comprises cyclosporin A, Atlas G-1096®, and Peanut oil.

Another embodiment comprises cyclosporin A, Atlas G-1292®, and Peanut oil.

Another embodiment comprises cyclosporin A, Atlas G-1293®, and Peanut oil.

Another embodiment comprises cyclosporin A, Atlas G-1300®, and Peanut oil.

Another embodiment comprises cyclosporin A, Atlas G-7076®, and Peanut oil.

Another embodiment comprises cyclosporin A, Capmul EMG®, and Peanut oil.

Another embodiment comprises cyclosporin A, Chemonic CO-40®, and Peanut oil.

Another embodiment comprises cyclosporin A, Chemonic LI-3®, and Peanut oil.

Another embodiment comprises cyclosporin A, Chemonic LI-7®, and Peanut oil.

Another embodiment comprises cyclosporin A, Cirrasol GM®, and Peanut oil.

Another embodiment comprises cyclosporin A, Cremophor CO 40®, and Peanut oil.

Another embodiment comprises cyclosporin A, Cremophor CO 410®, and Peanut oil.

Another embodiment comprises cyclosporin A, Cremophor EL®, and Peanut oil.

Another embodiment comprises cyclosporin A, Cremophor GC7®, and Peanut oil.

Another embodiment comprises cyclosporin A, Cremophor RH-40®, and Peanut oil.

Another embodiment comprises cyclosporin A, Crovol A-40®, and Peanut oil.

Another embodiment comprises cyclosporin A, Crovol A-70®, and Peanut oil.

Another embodiment comprises cyclosporin A, Crovol M-70®, and Peanut oil.

Another embodiment comprises cyclosporin A, Crovol PK-70®, and Peanut oil.

Another embodiment comprises cyclosporin A, Cutina E-24®, and Peanut oil.

Another embodiment comprises cyclosporin A, Dacospin 12-R®, and Peanut oil.

Another embodiment comprises cyclosporin A, Dehymuls HRE-7®, and Peanut oil.

Another embodiment comprises cyclosporin A, DeSonic 30C®, and Peanut oil.

Another embodiment comprises cyclosporin A, DeSonic 36C®, and Peanut oil.

Another embodiment comprises cyclosporin A, DeSonic 40C®, and Peanut oil.

Another embodiment comprises cyclosporin A, Durfax 60®, and Peanut oil.

Another embodiment comprises cyclosporin A, Dunax 65®, and Peanut oil.

Another embodiment comprises cyclosporin A, Durfax 80®, and Peanut oil.

Another embodiment comprises cyclosporin A, Durfax EOM®, and Peanut oil.

Another embodiment comprises cyclosporin A, Eccoterge NF-2®, and Peanut oil.

Another embodiment comprises cyclosporin A, Emulpon CO-360®, and Peanut oil.

Another embodiment comprises cyclosporin A, Emulpon CO-550®, and Peanut oil.

Another embodiment comprises cyclosporin A, Emulsogen EL®, and Peanut oil.

Another embodiment comprises cyclosporin A, Emulsogen HCO 040®, and Peanut oil.

Another embodiment comprises cyclosporin A, Emulsogen HCO 060®, and Peanut oil.

Another embodiment comprises cyclosporin A, Emulsynt 1055®, and Peanut oil.

Another embodiment comprises cyclosporin A, Ethox 3095®, and Peanut oil.

Another embodiment comprises cyclosporin A, Eumulgin RO-40®, and Peanut oil.

Another embodiment comprises cyclosporin A, Genapol G-260®, and Peanut oil.

Another embodiment comprises cyclosporin A, Glycosperse L-20®, and Peanut oil.

Another embodiment comprises cyclosporin A, Glycosperse O-5®, and Peanut oil.

Another embodiment comprises cyclosporin A, Glycosperse O-20®, and Peanut oil.

Another embodiment comprises cyclosporin A, Glycosperse O-20 FG®, and Peanut oil.

Another embodiment comprises cyclosporin A, Glycosperse S-20®, and Peanut oil.

Another embodiment comprises cyclosporin A, Glycosperse S-20 FG®, and Peanut oil.

Another embodiment comprises cyclosporin A, Glycosperse TS-20®, and Peanut oil.

Another embodiment comprises cyclosporin A, Glycosperse TS-20 FG®, and Peanut oil.

Another embodiment comprises cyclosporin A, Hetan SL®, and Peanut oil.

Another embodiment comprises cyclosporin A, Hetan SO®, and Peanut oil.

Another embodiment comprises cyclosporin A, Hetan SS®, and Peanut oil.

Another embodiment comprises cyclosporin A, Hetoxide C-2®, and Peanut oil.

Another embodiment comprises cyclosporin A, Hetoxide C-9®, and Peanut oil.

Another embodiment comprises cyclosporin A, Hetoxide C-15®, and Peanut oil.

Another embodiment comprises cyclosporin A, Hetoxide C-25®, and Peanut oil.

Another embodiment comprises cyclosporin A, Hetoxide C-40®, and Peanut oil.

Another embodiment comprises cyclosporin A, Hetoxide C-200®, and Peanut oil.

Another embodiment comprises cyclosporin A, Hetoxide C-200-50%®, and Peanut oil.

Another embodiment comprises cyclosporin A, Hetoxide GC-30®, and Peanut oil.

Another embodiment comprises cyclosporin A, Hetoxide HC-60®, and Peanut oil.

Another embodiment comprises cyclosporin A, Ice No. 2®, and Peanut oil.

Another embodiment comprises cyclosporin A, Incrocas 30/40®, and Peanut oil.

Another embodiment comprises cyclosporin A, Lexol EC®, and Peanut oil.

Another embodiment comprises cyclosporin A, Lexol EO®, and Peanut oil.

Another embodiment comprises cyclosporin A, Lipocol HCO-40®, and Peanut oil.

Another embodiment comprises cyclosporin A, Lipocol HCO-60®, and Peanut oil.

Another embodiment comprises cyclosporin A, Lipocol O-3 Special®, and Peanut oil.

Another embodiment comprises cyclosporin A, Lipopeg 2-L®, and Peanut oil.

Another embodiment comprises cyclosporin A, Lipopeg 4-DO®, and Peanut oil.

Another embodiment comprises cyclosporin A, Lipopeg 4-DS®, and Peanut oil.

Another embodiment comprises cyclosporin A, Lipovol GTB®, and Peanut oil.

Another embodiment comprises cyclosporin A, Lonzest SML-20®, and Peanut oil.

Another embodiment comprises cyclosporin A, Lonzest SMO-20®, and Peanut oil.

Another embodiment comprises cyclosporin A, Lonzest SMS-20®, and Peanut oil.

Another embodiment comprises cyclosporin A, Lonzest STO-20®, and Peanut oil.

Another embodiment comprises cyclosporin A, Lonzest STS-20®, and Peanut oil.

Another embodiment comprises cyclosporin A, Lumulse GR-40®, and Peanut oil.

Another embodiment comprises cyclosporin A, Lumulse GRH-40®, and Peanut oil.

Another embodiment comprises cyclosporin A, Lumulse POE (7) GML®, and Peanut oil.

Another embodiment comprises cyclosporin A, Lumulse POE (12) Glyc®, and Peanut oil.

Another embodiment comprises cyclosporin A, Lumulse POE (40) MS KP®, and Peanut oil.

Another embodiment comprises cyclosporin A, Marlowet 4750®, and Peanut oil.

Another embodiment comprises cyclosporin A, Marlowet LVS®, and Peanut oil.

Another embodiment comprises cyclosporin A, Marlowet R 11®, and Peanut oil.

Another embodiment comprises cyclosporin A, Marlowet R 40®, and Peanut oil.

Another embodiment comprises cyclosporin A, Mazol 80 MGK®, and Peanut oil.

Another embodiment comprises cyclosporin A, Nonionic Emulsifier T-9®, and Peanut oil.

Another embodiment comprises cyclosporin A, Oronal LCG®, and Peanut oil.

Another embodiment comprises cyclosporin A, Polyderm PPI-CO-200®, and Peanut oil.

Another embodiment comprises cyclosporin A, Polyderm PPI-CO-40®, and Peanut oil.

Another embodiment comprises cyclosporin A, Rewoderm LI 520-70®, and Peanut oil.

Another embodiment comprises cyclosporin A, Ritapeg 150 DS®, and Peanut oil.

Another embodiment comprises cyclosporin A, Sottigen 767®, and Peanut oil.

Another embodiment comprises cyclosporin A, Surfactol 318®, and Peanut oil.

Another embodiment comprises cyclosporin A, Surfactol 365®, and Peanut oil.

Another embodiment comprises cyclosporin A, Syn Lube 107®, and Peanut oil.

Another embodiment comprises cyclosporin A, Syn Lube 728®, and Peanut oil.

Another embodiment comprises cyclosporin A, Syn Lube 1632H®, and Peanut oil.

Another embodiment comprises cyclosporin A, Syn Lube 6277-A®, and Peanut oil.

Another embodiment comprises cyclosporin A, T-Det C-20®, and Peanut oil.

Another embodiment comprises cyclosporin A, T-Det C-40®, and Peanut oil.

Another embodiment comprises cyclosporin A, Tally 100 Plus®, and Peanut oil.

Another embodiment comprises cyclosporin A, Uniperol EL®, and Peanut oil.

Another embodiment comprises cyclosporin A, a Fatty Ester, and Peanut oil.

Another embodiment comprises cyclosporin A, Actralube-Syn 147®, and Peanut oil.

Another embodiment comprises cyclosporin A, Atlas G-1556®, and Peanut oil.

Another embodiment comprises cyclosporin A, Atlas G-1564®, and Peanut oil.

Another embodiment comprises cyclosporin A, Atlasol Base Oil S®, and Peanut oil.

Another embodiment comprises cyclosporin A, Base ML®, and Peanut oil.

Another embodiment comprises cyclosporin A, Base MT®, and Peanut oil.

Another embodiment comprises cyclosporin A, Cerasynt 303®, and Peanut oil.

Another embodiment comprises cyclosporin A, Dermol 1012®, and Peanut oil.

Another embodiment comprises cyclosporin A, Kemester 4000®, and Peanut oil.

Another embodiment comprises cyclosporin A, Lactipol S®, and Peanut oil.

Another embodiment comprises cyclosporin A, Magrabar PGO®, and Peanut oil.

Another embodiment comprises cyclosporin A, Mayco Base BFO®, and Peanut oil.

Another embodiment comprises cyclosporin A, Methyl Linoleate®, and Peanut oil.

Another embodiment comprises cyclosporin A, Pationic 122A®, and Peanut oil.

Another embodiment comprises cyclosporin A, Pationic 138C®, and Peanut oil.

Another embodiment comprises cyclosporin A, Pationic CSL®, and Peanut oil.

Another embodiment comprises cyclosporin A, Pationic ISL®, and Peanut oil.

Another embodiment comprises cyclosporin A, Pationic SBL®, and Peanut oil.

Another embodiment comprises cyclosporin A, Pationic SSL®, and Peanut oil.

Another embodiment comprises cyclosporin A, Ritasol®, and Peanut oil.

Another embodiment comprises cyclosporin A, Tego Alkanol CS 20®, and Peanut oil.

Another embodiment comprises cyclosporin A, Tego Alkanol L23 P®, and Peanut oil.

Another embodiment comprises cyclosporin A, Tego Alkanol S2®, and Peanut oil.

Another embodiment comprises cyclosporin A, Tego Alkanol S20 P®, and Peanut oil.

Another embodiment comprises cyclosporin A, Triemulsifier 600 MS®, and Peanut oil.

Another embodiment comprises cyclosporin A, a Glycerol Esters, and Peanut oil.

Another embodiment comprises cyclosporin A, Agro #9 Wint SBO®, and Peanut oil.

Another embodiment comprises cyclosporin A, Ahcovel Base 700®, and Peanut oil.

Another embodiment comprises cyclosporin A, Aldo HMS FG®, and Peanut oil.

Another embodiment comprises cyclosporin A, Aldo MLD®, and Peanut oil.

Another embodiment comprises cyclosporin A, Aldo MLD FG®, and Peanut oil.

Another embodiment comprises cyclosporin A, Aldo MO FG®, and Peanut oil.

Another embodiment comprises cyclosporin A, Aldo MS®, and Peanut oil.

Another embodiment comprises cyclosporin A, Aldo MS FG®, and Peanut oil.

Another embodiment comprises cyclosporin A, Aldo MS LG FG®, and Peanut oil.

Another embodiment comprises cyclosporin A, Aldo MSD®, and Peanut oil.

Another embodiment comprises cyclosporin A, Aldo MSD FG®, and Peanut oil.

Another embodiment comprises cyclosporin A, Aldosperse O-20 FG®, and Peanut oil.

Another embodiment comprises cyclosporin A, Aldosperse TS-20 FG®, and Peanut oil.

Another embodiment comprises cyclosporin A, Aldosperse TS-40 FG®, and Peanut oil.

Another embodiment comprises cyclosporin A, Arlacel 165®, and Peanut oil.

Another embodiment comprises cyclosporin A, Arlacel 186®, and Peanut oil.

Another embodiment comprises cyclosporin A, Capmul GMO®, and Peanut oil.

Another embodiment comprises cyclosporin A, Capmul GMS®, and Peanut oil.

Another embodiment comprises cyclosporin A, Caprol 3GO®, and Peanut oil.

Another embodiment comprises cyclosporin A, Caprol 3GVS®, and Peanut oil.

Another embodiment comprises cyclosporin A, Caprol 6G2S®, and Peanut oil.

Another embodiment comprises cyclosporin A, Caprol 10G40®, and Peanut oil.

Another embodiment comprises cyclosporin A, Caprol 10G100®, and Peanut oil.

Another embodiment comprises cyclosporin A, Caprol ET®, and Peanut oil.

Another embodiment comprises cyclosporin A, Caprol PGE860®, and Peanut oil.

Another embodiment comprises cyclosporin A, Cerasynt 945®, and Peanut oil.

Another embodiment comprises cyclosporin A, Cerasynt GMS®, and Peanut oil.

Another embodiment comprises cyclosporin A, Cerasynt Q®, and Peanut oil.

Another embodiment comprises cyclosporin A, Cerasynt SD®, and Peanut oil.

Another embodiment comprises cyclosporin A, Cerasynt WM®, and Peanut oil.

Another embodiment comprises cyclosporin A, Chemsperse 14®, and Peanut oil.

Another embodiment comprises cyclosporin A, Cremophor GO-32®, and Peanut oil.

Another embodiment comprises cyclosporin A, Cremophor GS11®, and Peanut oil.

Another embodiment comprises cyclosporin A, Cremophor GS-32®, and Peanut oil.

Another embodiment comprises cyclosporin A, Cutina KD-16®, and Peanut oil.

Another embodiment comprises cyclosporin A, Dehymuls PGPH®, and Peanut oil.

Another embodiment comprises cyclosporin A, Dermol DGDIS®, and Peanut oil.

Another embodiment comprises cyclosporin A, Dermol DGMIS®, and Peanut oil.

Another embodiment comprises cyclosporin A, Dermol G-76®, and Peanut oil.

Another embodiment comprises cyclosporin A, Dermol G-7DI®, and Peanut oil.

Another embodiment comprises cyclosporin A, Dermol NGDI®, and Peanut oil.

Another embodiment comprises cyclosporin A, Dermolan GLH®, and Peanut oil.

Another embodiment comprises cyclosporin A, Drewmulse GMO®, and Peanut oil.

Another embodiment comprises cyclosporin A, Drewpol 3-5-M®, and Peanut oil.

Another embodiment comprises cyclosporin A, Durlac 100 W®, and Peanut oil.

Another embodiment comprises cyclosporin A, Dur-Lo®, and Peanut oil.

Another embodiment comprises cyclosporin A, Dynasan 118®, and Peanut oil.

Another embodiment comprises cyclosporin A, EC-25®, and Peanut oil.

Another embodiment comprises cyclosporin A, EM 40®, and Peanut oil.

Another embodiment comprises cyclosporin A, Emerest 2400®, and Peanut oil.

Another embodiment comprises cyclosporin A, Emerest 2452®, and Peanut oil.

Another embodiment comprises cyclosporin A, Empilan G-26®, and Peanut oil.

Another embodiment comprises cyclosporin A, Genapol TSM®, and Peanut oil.

Another embodiment comprises cyclosporin A, Hostacerin DGI®, and Peanut oil.

Another embodiment comprises cyclosporin A, Hostacerin DGL®, and Peanut oil.

Another embodiment comprises cyclosporin A, Hostacerin DGMS®, and Peanut oil.

Another embodiment comprises cyclosporin A, Hostacerin DGSB®, and Peanut oil.

Another embodiment comprises cyclosporin A, Ice No. 2®, and Peanut oil.

Another embodiment comprises cyclosporin A, Imwitor 742®, and Peanut oil.

Another embodiment comprises cyclosporin A, Imwitor 780 K®, and Peanut oil.

Another embodiment comprises cyclosporin A, lmwitor 960 Flakes®, and Peanut oil.

Another embodiment comprises cyclosporin A, Isolan Gl 34®, and Peanut oil.

Another embodiment comprises cyclosporin A, Isolan GO 33®, and Peanut oil.

Another embodiment comprises cyclosporin A, Kemester 1000®, and Peanut oil.

Another embodiment comprises cyclosporin A, Kemester 2000®, and Peanut oil.

Another embodiment comprises cyclosporin A, Kemester 2000®, and Peanut oil.

Another embodiment comprises cyclosporin A, Kemester 6000SE®, and Peanut oil.

Another embodiment comprises cyclosporin A, Lamecreme DGE 18®, and Peanut oil.

Another embodiment comprises cyclosporin A, Lexemul 515®, and Peanut oil.

Another embodiment comprises cyclosporin A, Lexemul 561®, and Peanut oil.

Another embodiment comprises cyclosporin A, Lexemul AR®, and Peanut oil.

Another embodiment comprises cyclosporin A, Lexemul AS®, and Peanut oil.

Another embodiment comprises cyclosporin A, Lexemul GDL®, and Peanut oil.

Another embodiment comprises cyclosporin A, Lexemul T®, and Peanut oil.

Another embodiment comprises cyclosporin A, Lipomulse 165®, and Peanut oil.

Another embodiment comprises cyclosporin A, Lumulse GML K®, and Peanut oil.

Another embodiment comprises cyclosporin A, Lumulse GMO K®, and Peanut oil.

Another embodiment comprises cyclosporin A, Lumulse GMR K®, and Peanut oil.

Another embodiment comprises cyclosporin A, Lumulse GMT K®, and Peanut oil.

Another embodiment comprises cyclosporin A, Magrabar GMC®, and Peanut oil.

Another embodiment comprises cyclosporin A, Magrabar GMO-CK®, and Peanut oil.

Another embodiment comprises cyclosporin A, Magrabar MDG-5050®, and Peanut oil.

Another embodiment comprises cyclosporin A, Magrabar PGO-315®, and Peanut oil.

Another embodiment comprises cyclosporin A, Magrabar PGO-1010®, and Peanut oil.

Another embodiment comprises cyclosporin A, Mazol 300K®, and Peanut oil.

Another embodiment comprises cyclosporin A, Mazol GMO-K®, and Peanut oil.

Another embodiment comprises cyclosporin A, Mazol GMS-K®, and Peanut oil.

Another embodiment comprises cyclosporin A, Mazol PG031-K®, and Peanut oil.

Another embodiment comprises cyclosporin A, Miglyol 812®, and Peanut oil.

Another embodiment comprises cyclosporin A, Norfox 165C®, and Peanut oil.

Another embodiment comprises cyclosporin A, Schercemol GMIS®, and Peanut oil.

Another embodiment comprises cyclosporin A, Tegin®, and Peanut oil.

Another embodiment comprises cyclosporin A, Tegin 4100 Pellets®, and Peanut oil.

Another embodiment comprises cyclosporin A, Tegin M Pellets®, and Peanut oil.

Another embodiment comprises cyclosporin A, Tegin OV®, and Peanut oil.

Another embodiment comprises cyclosporin A, Teginacid H®, and Peanut oil.

Another embodiment comprises cyclosporin A, Tego Cosmo P813®, and Peanut oil.

Another embodiment comprises cyclosporin A, Wickenol 535®, and Peanut oil.

Another embodiment comprises cyclosporin A, Witconol 14®, and Peanut oil.

Another embodiment comprises cyclosporin A, Witconol 14®, and Peanut oil.

Another embodiment comprises cyclosporin A, Witconol 14F®, and Peanut oil.

Another embodiment comprises cyclosporin A, Witconol 18L®, and Peanut oil.

Another embodiment comprises cyclosporin A, Witconol GOT®, and Peanut oil.

Another embodiment comprises cyclosporin A, Witconol MST®, and Peanut oil.

Another embodiment comprises cyclosporin A, Witconol RHT®, and Peanut oil.

Another embodiment comprises cyclosporin A, a Glycol Ester, and Peanut oil.

Another embodiment comprises cyclosporin A, Alkamuls 600 DO®, and Peanut oil.

Another embodiment comprises cyclosporin A, Alkamuls SEG®, and Peanut oil.

Another embodiment comprises cyclosporin A, Atlas EM-2®, and Peanut oil.

Another embodiment comprises cyclosporin A, Cerasynt IP®, and Peanut oil.

Another embodiment comprises cyclosporin A, Cerasynt M®, and Peanut oil.

Another embodiment comprises cyclosporin A, Cerasynt MN®, and Peanut oil.

Another embodiment comprises cyclosporin A, Cerasynt PA®, and Peanut oil.

Another embodiment comprises cyclosporin A, Chemsperse EGDS®, and Peanut oil.

Another embodiment comprises cyclosporin A, Chemsperse EGMS®, and Peanut oil.

Another embodiment comprises cyclosporin A, Colonial Monolaurin®, and Peanut oil.

Another embodiment comprises cyclosporin A, DeMuls SGE-95®, and Peanut oil.

Another embodiment comprises cyclosporin A, Eccoterge 200®, and Peanut oil.

Another embodiment comprises cyclosporin A, Emerest 2380®, and Peanut oil.

Another embodiment comprises cyclosporin A, Ethox 2610®, and Peanut oil.

Another embodiment comprises cyclosporin A, Ethox DO-9®, and Peanut oil.

Another embodiment comprises cyclosporin A, Ethox DO-14®, and Peanut oil.

Another embodiment comprises cyclosporin A, Ethox SO-9®, and Peanut oil.

Another embodiment comprises cyclosporin A, Fizul MD-318®, and Peanut oil.

Another embodiment comprises cyclosporin A, Genapol EGDS-VHP®, and Peanut oil.

Another embodiment comprises cyclosporin A, Genapol TS Powder®, and Peanut oil.

Another embodiment comprises cyclosporin A, Hostacerin WO®, and Peanut oil.

Another embodiment comprises cyclosporin A, Inversol 140®, and Peanut oil.

Another embodiment comprises cyclosporin A, Kemester 104®, and Peanut oil.

Another embodiment comprises cyclosporin A, Kemester 205®, and Peanut oil.

Another embodiment comprises cyclosporin A, Kemester 226®, and Peanut oil.

Another embodiment comprises cyclosporin A, Kemester 5221SE®, and Peanut oil.

Another embodiment comprises cyclosporin A, Kemester EGDS®, and Peanut oil.

Another embodiment comprises cyclosporin A, Lexemul EGDS®, and Peanut oil.

Another embodiment comprises cyclosporin A, Lexemul EGMS®, and Peanut oil.

Another embodiment comprises cyclosporin A, Lexemul P®, and Peanut oil.

Another embodiment comprises cyclosporin A, Lipo DGLS®, and Peanut oil.

Another embodiment comprises cyclosporin A, Lipo EGDS®, and Peanut oil.

Another embodiment comprises cyclosporin A, Lipo PGMS®, and Peanut oil.

Another embodiment comprises cyclosporin A, Liposorb S-4®, and Peanut oil.

Another embodiment comprises cyclosporin A, Liposorb TO-20®, and Peanut oil.

Another embodiment comprises cyclosporin A, Lumulse PGO®, and Peanut oil.

Another embodiment comprises cyclosporin A, Mackester EGDS®, and Peanut oil.

Another embodiment comprises cyclosporin A, Mackester EGMS®, and Peanut oil.

Another embodiment comprises cyclosporin A, Mackester GSTP®, and Peanut oil.

Another embodiment comprises cyclosporin A, Mackester Series®, and Peanut oil.

Another embodiment comprises cyclosporin A, Magrabar PDG-50®, and Peanut oil.

Another embodiment comprises cyclosporin A, Mapeg 6000 DS®, and Peanut oil.

Another embodiment comprises cyclosporin A, Marlowet 4702®, and Peanut oil.

Another embodiment comprises cyclosporin A, Monalube 305®, and Peanut oil.

Another embodiment comprises cyclosporin A, Monalube 310®, and Peanut oil.

Another embodiment comprises cyclosporin A, Monalube 315®, and Peanut oil.

Another embodiment comprises cyclosporin A, Monalube 320®, and Peanut oil.

Another embodiment comprises cyclosporin A, Monalube 325®, and Peanut oil.

Another embodiment comprises cyclosporin A, Monalube 330®, and Peanut oil.

Another embodiment comprises cyclosporin A, Naturechem PGHS®, and Peanut oil.

Another embodiment comprises cyclosporin A, Polycastorol PLO-840®, and Peanut oil.

Another embodiment comprises cyclosporin A, Polytex 10M®, and Peanut oil.

Another embodiment comprises cyclosporin A, Ritasynt IP®, and Peanut oil.

Another embodiment comprises cyclosporin A, Ross Chem PEG 600 DT®, and Peanut oil.

Another embodiment comprises cyclosporin A, Schercemol PGMS®, and Peanut oil.

Another embodiment comprises cyclosporin A, Sponto H-44C®, and Peanut oil.

Another embodiment comprises cyclosporin A, Tegin G®, and Peanut oil.

Another embodiment comprises cyclosporin A, Witbreak DGE-182®, and Peanut oil.

Another embodiment comprises cyclosporin A, Witbreak DGE-182®, and Peanut oil.

Another embodiment comprises cyclosporin A, Witbreak DRA-21®, and Peanut oil.

Another embodiment comprises cyclosporin A, Witbreak DRA-21®, and Peanut oil.

Another embodiment comprises cyclosporin A, Witbreak DRA-50®, and Peanut oil.

Another embodiment comprises cyclosporin A, Witbreak DRA-50®, and Peanut oil.

Another embodiment comprises cyclosporin A, Witconol F26-46®, and Peanut oil.

Another embodiment comprises cyclosporin A, Witconol H-32®, and Peanut oil.

Another embodiment comprises cyclosporin A, Witconol H-33®, and Peanut oil.

Another embodiment comprises cyclosporin A, Witconol H-35A®, and Peanut oil.

Another embodiment comprises cyclosporin A, Witconol RHP®, and Peanut oil.

Another embodiment comprises cyclosporin A, a Lanolin-based Derivative, and Peanut oil.

Another embodiment comprises cyclosporin A, Amerchol CAB®, and Peanut oil.

Another embodiment comprises cyclosporin A, Amerchol L-101®, and Peanut oil.

Another embodiment comprises cyclosporin A, Amerlate LFA-LO®, and Peanut oil.

Another embodiment comprises cyclosporin A, Amerlate P®, and Peanut oil.

Another embodiment comprises cyclosporin A, Barre Common Degras®, and Peanut oil.

Another embodiment comprises cyclosporin A, Cholesterol NF®, and Peanut oil.

Another embodiment comprises cyclosporin A, Crodalan AWS®, and Peanut oil.

Another embodiment comprises cyclosporin A, Crodalan LA®, and Peanut oil.

Another embodiment comprises cyclosporin A, Emery 1650®, and Peanut oil.

Another embodiment comprises cyclosporin A, Emery 1650®, and Peanut oil.

Another embodiment comprises cyclosporin A, Emery 1740®, and Peanut oil.

Another embodiment comprises cyclosporin A, Emery 1740®, and Peanut oil.

Another embodiment comprises cyclosporin A, Forlan 500®, and Peanut oil.

Another embodiment comprises cyclosporin A, Forlan L®, and Peanut oil.

Another embodiment comprises cyclosporin A, Laneto 50®, and Peanut oil.

Another embodiment comprises cyclosporin A, Laneto 100®, and Peanut oil.

Another embodiment comprises cyclosporin A, Laneto AWS®, and Peanut oil.

Another embodiment comprises cyclosporin A, Lanfrax 1776®, and Peanut oil.

Another embodiment comprises cyclosporin A, Lanfrax 1776®, and Peanut oil.

Another embodiment comprises cyclosporin A, Lanogel 21®, and Peanut oil.

Another embodiment comprises cyclosporin A, Lipolan®, and Peanut oil.

Another embodiment comprises cyclosporin A, Lipolan 31®, and Peanut oil.

Another embodiment comprises cyclosporin A, OHlan®, and Peanut oil.

Another embodiment comprises cyclosporin A, Polychol 5®, and Peanut oil.

Another embodiment comprises cyclosporin A, Polychol 15®, and Peanut oil.

Another embodiment comprises cyclosporin A, Ritacetyl®, and Peanut oil.

Another embodiment comprises cyclosporin A, Ritachol®, and Peanut oil.

Another embodiment comprises cyclosporin A, Ritahydrox®, and Peanut oil.

Another embodiment comprises cyclosporin A, Ritalafa®, and Peanut oil.

Another embodiment comprises cyclosporin A, Ritalan®, and Peanut oil.

Another embodiment comprises cyclosporin A, Ritalan AWS®, and Peanut oil.

Another embodiment comprises cyclosporin A, Ritalan C®, and Peanut oil.

Another embodiment comprises cyclosporin A, Ritawax®, and Peanut oil.

Another embodiment comprises cyclosporin A, Ritawax AEC®, and Peanut oil.

Another embodiment comprises cyclosporin A, Ritawax ALA®, and Peanut oil.

Another embodiment comprises cyclosporin A, Solan/Solan 50/Super Solan®, and Peanut oil.

Another embodiment comprises cyclosporin A, Super Hartolan/Hartolan®, and Peanut oil.

Another embodiment comprises cyclosporin A, Supersat AWS-4®, and Peanut oil.

Another embodiment comprises cyclosporin A, Supersat AWS-24®, and Peanut oil.

Another embodiment comprises cyclosporin A, a Lecithin or Lecithin Derivative, and Peanut oil.

Another embodiment comprises cyclosporin A, Alcolec®, and Peanut oil.

Another embodiment comprises cyclosporin A, Lecithin®, and Peanut oil.

Another embodiment comprises cyclosporin A, Lexin K®, and Peanut oil.

Another embodiment comprises cyclosporin A, Natipide®, and Peanut oil.

Another embodiment comprises cyclosporin A, a Lignin or Lignin Derivative, and Peanut oil.

Another embodiment comprises cyclosporin A, Diwatex XP 9®, and Peanut oil.

Another embodiment comprises cyclosporin A, Dynasperse LCD®, and Peanut oil.

Another embodiment comprises cyclosporin A, Indulin SAL®, and Peanut oil.

Another embodiment comprises cyclosporin A, Indulin W-1®, and Peanut oil.

Another embodiment comprises cyclosporin A, Indulin W-5®, and Peanut oil.

Another embodiment comprises cyclosporin A, Lignosol FTA®, and Peanut oil.

Another embodiment comprises cyclosporin A, Lignosol SFX-65®, and Peanut oil.

Another embodiment comprises cyclosporin A, Marasperse 52 CP®, and Peanut oil.

Another embodiment comprises cyclosporin A, Marasperse AG®, and Peanut oil.

Another embodiment comprises cyclosporin A, Marasperse CBOS-4®, and Peanut oil.

Another embodiment comprises cyclosporin A, Ufoxane 2®, and Peanut oil.

Another embodiment comprises cyclosporin A, a Methyl Ester, and Peanut oil.

Another embodiment comprises cyclosporin A, E.B. Cleaner AK®, and Peanut oil.

Another embodiment comprises cyclosporin A, Oleocal ME-70®, and Peanut oil.

Another embodiment comprises cyclosporin A, Trade Name Company®, and Peanut oil.

Another embodiment comprises cyclosporin A, Methyl Esters (cont'd)®, and Peanut oil.

Another embodiment comprises cyclosporin A, Oleocal ME-92®, and Peanut oil.

Another embodiment comprises cyclosporin A, Oleocal ME-112®, and Peanut oil.

Another embodiment comprises cyclosporin A, Oleocal ME-130®, and Peanut oil.

Another embodiment comprises cyclosporin A, a Monoglyceride or a Derivative thereof, and Peanut oil.

Another embodiment comprises cyclosporin A, Dynacet 211®, and Peanut oil.

Another embodiment comprises cyclosporin A, Hetsorb S-20®, and Peanut oil.

Another embodiment comprises cyclosporin A, lmwitor 191®, and Peanut oil.

Another embodiment comprises cyclosporin A, lmwitor 370®, and Peanut oil.

Another embodiment comprises cyclosporin A, lmwitor 375®, and Peanut oil.

Another embodiment comprises cyclosporin A, lmwitor 900®, and Peanut oil.

Another embodiment comprises cyclosporin A, lmwitor 945®, and Peanut oil.

Another embodiment comprises cyclosporin A, Imwitor 2020®, and Peanut oil.

Another embodiment comprises cyclosporin A, Kemester 5500®, and Peanut oil.

Another embodiment comprises cyclosporin A, Kemester 6000®, and Peanut oil.

Another embodiment comprises cyclosporin A, Magrabar GMC®, and Peanut oil.

Another embodiment comprises cyclosporin A, Magrabar GMO-CK®, and Peanut oil.

Another embodiment comprises cyclosporin A, Magrabar GPC-10®, and Peanut oil.

Another embodiment comprises cyclosporin A, Magrabar MDG-5050®, and Peanut oil.

Another embodiment comprises cyclosporin A, Monalube 335®, and Peanut oil.

Another embodiment comprises cyclosporin A, Monoglycerides Glyceryl Monestearate Archer Daniels Midland Company®, and Peanut oil.

Another embodiment comprises cyclosporin A, Rita GMS®, and Peanut oil.

Another embodiment comprises cyclosporin A, Ritamulse SCG®, and Peanut oil.

Another embodiment comprises cyclosporin A, Softigen 701®, and Peanut oil.

Another embodiment comprises cyclosporin A, Tally 100 Plus®, and Peanut oil.

Another embodiment comprises cyclosporin A, a Polyethylene Glycol, and Peanut oil.

Another embodiment comprises cyclosporin A, Emulgade PL 68/50®, and Peanut oil.

Another embodiment comprises cyclosporin A, Lumulse PEG®, and Peanut oil.

Another embodiment comprises cyclosporin A, Rhodasurf PEG-400®, and Peanut oil.

Another embodiment comprises cyclosporin A, Rhodasurf PEG-600®, and Peanut oil.

Another embodiment comprises cyclosporin A, Witconol PEG-400®, and Peanut oil.

Another embodiment comprises cyclosporin A, a Polymeric Surfactant, and Peanut oil.

Another embodiment comprises cyclosporin A, Acritamer PNC-EG®, and Peanut oil.

Another embodiment comprises cyclosporin A, Ag—Rho DEP-775®, and Peanut oil.

Another embodiment comprises cyclosporin A, APG 325N Glycoside®, and Peanut oil.

Another embodiment comprises cyclosporin A, Aristoflex AVC®, and Peanut oil.

Another embodiment comprises cyclosporin A, Aristoflex HMB®, and Peanut oil.

Another embodiment comprises cyclosporin A, Burco NPS-225®, and Peanut oil.

Another embodiment comprises cyclosporin A, Burco NPS-816®, and Peanut oil.

Another embodiment comprises cyclosporin A, Chemccinate 5603®, and Peanut oil.

Another embodiment comprises cyclosporin A, Cosmedia Guar C-261 N®, and Peanut oil.

Another embodiment comprises cyclosporin A, Gantrez S-95®, and Peanut oil.

Another embodiment comprises cyclosporin A, Glucopon 220 UP®, and Peanut oil.

Another embodiment comprises cyclosporin A, Glucopon 225 DK®, and Peanut oil.

Another embodiment comprises cyclosporin A, Glucopon 425 N®, and Peanut oil.

Another embodiment comprises cyclosporin A, Glucopon 600 UP®, and Peanut oil.

Another embodiment comprises cyclosporin A, Glucopon 625 UP®, and Peanut oil.

Another embodiment comprises cyclosporin A, Pemulen 1621®, and Peanut oil.

Another embodiment comprises cyclosporin A, Pemulen 1622®, and Peanut oil.

Another embodiment comprises cyclosporin A, Pemulen TR-1®, and Peanut oil.

Another embodiment comprises cyclosporin A, Pemulen TR-2®, and Peanut oil.

Another embodiment comprises cyclosporin A, Plantacare 818®, and Peanut oil.

Another embodiment comprises cyclosporin A, Plantapon LGC Sorb®, and Peanut oil.

Another embodiment comprises cyclosporin A, Plantaren 1200N®, and Peanut oil.

Another embodiment comprises cyclosporin A, Plantaren 2000N®, and Peanut oil.

Another embodiment comprises cyclosporin A, Viscolam AT 64®, and Peanut oil.

Another embodiment comprises cyclosporin A, Viscolam AT 64P®, and Peanut oil.

Another embodiment comprises cyclosporin A, Viscolam AT 100®, and Peanut oil.

Another embodiment comprises cyclosporin A, Viscolam MAC 7®, and Peanut oil.

Another embodiment comprises cyclosporin A, Viscolam SMC 20®, and Peanut oil.

Another embodiment comprises cyclosporin A, Witbreak RTC-323®, and Peanut oil.

Another embodiment comprises cyclosporin A, WSI 3700®, and Peanut oil.

Another embodiment comprises cyclosporin A, a Propoxylated or Ethoxylated Fatty Acid, Alcohol, or Alkyl Phenol, and Peanut oil.

Another embodiment comprises cyclosporin A, Antarox AA-60®, and Peanut oil.

Another embodiment comprises cyclosporin A, Antarox LF-224®, and Peanut oil.

Another embodiment comprises cyclosporin A, Burcomul DFE-45®, and Peanut oil.

Another embodiment comprises cyclosporin A, Burcoterge LFE-1000®, and Peanut oil.

Another embodiment comprises cyclosporin A, Chemal LF-25B®, and Peanut oil.

Another embodiment comprises cyclosporin A, Chemal LF-40B®, and Peanut oil.

Another embodiment comprises cyclosporin A, Dehypon LS-36®, and Peanut oil.

Another embodiment comprises cyclosporin A, Dehypon LS-36®, and Peanut oil.

Another embodiment comprises cyclosporin A, Dehypon LS-54®, and Peanut oil.

Another embodiment comprises cyclosporin A, Dehypon LS-54®, and Peanut oil.

Another embodiment comprises cyclosporin A, Delonic 100 VLF®, and Peanut oil.

Another embodiment comprises cyclosporin A, Delonic LF-60 MOD®, and Peanut oil.

Another embodiment comprises cyclosporin A, Empiderm B®, and Peanut oil.

Another embodiment comprises cyclosporin A, Ethylan 1206®, and Peanut oil.

Another embodiment comprises cyclosporin A, Ethylan NS-500K®, and Peanut oil.

Another embodiment comprises cyclosporin A, Ethylan NS-500LQ®, and Peanut oil.

Another embodiment comprises cyclosporin A, Genapol 1392®, and Peanut oil.

Another embodiment comprises cyclosporin A, Genapol 2317®, and Peanut oil.

Another embodiment comprises cyclosporin A, Genapol 26EP710®, and Peanut oil.

Another embodiment comprises cyclosporin A, Genapol EP 1022®, and Peanut oil.

Another embodiment comprises cyclosporin A, Genapol EP 1024®, and Peanut oil.

Another embodiment comprises cyclosporin A, Genapol EP 6068®, and Peanut oil.

Another embodiment comprises cyclosporin A, Genapol NP915®, and Peanut oil.

Another embodiment comprises cyclosporin A, Kieralon MFB®, and Peanut oil.

Another embodiment comprises cyclosporin A, Lumisolve CSA-80 V®, and Peanut oil.

Another embodiment comprises cyclosporin A, Marlowet 5001®, and Peanut oil.

Another embodiment comprises cyclosporin A, Marlox FK 64®, and Peanut oil.

Another embodiment comprises cyclosporin A, Marlox MO 124®, and Peanut oil.

Another embodiment comprises cyclosporin A, Marlox S 58®, and Peanut oil.

Another embodiment comprises cyclosporin A, Nonatell 1003®, and Peanut oil.

Another embodiment comprises cyclosporin A, Nonatell 1038®, and Peanut oil.

Another embodiment comprises cyclosporin A, Nonatell 1052®, and Peanut oil.

Another embodiment comprises cyclosporin A, Nonatell 1061®, and Peanut oil.

Another embodiment comprises cyclosporin A, Nonatell 1075®, and Peanut oil.

Another embodiment comprises cyclosporin A, Nonatell 1088®, and Peanut oil.

Another embodiment comprises cyclosporin A, Nonatell 1123®, and Peanut oil.

Another embodiment comprises cyclosporin A, Nonatell 1153®, and Peanut oil.

Another embodiment comprises cyclosporin A, Nonatell 1161®, and Peanut oil.

Another embodiment comprises cyclosporin A, Nonatell 1172®, and Peanut oil.

Another embodiment comprises cyclosporin A, Nonatell 1181®, and Peanut oil.

Another embodiment comprises cyclosporin A, Norfox 36®, and Peanut oil.

Another embodiment comprises cyclosporin A, Procetyl AWS®, and Peanut oil.

Another embodiment comprises cyclosporin A, Sandoxylate SX 412®, and Peanut oil.

Another embodiment comprises cyclosporin A, Sandoxylate SX 418®, and Peanut oil.

Another embodiment comprises cyclosporin A, Surfonic JL-80X®, and Peanut oil.

Another embodiment comprises cyclosporin A, Surfonic JL-80X-B1®, and Peanut oil.

Another embodiment comprises cyclosporin A, Surfonic L4-29X®, and Peanut oil.

Another embodiment comprises cyclosporin A, Surfonic LF®, and Peanut oil.

Another embodiment comprises cyclosporin A, T-Det A826®, and Peanut oil.

Another embodiment comprises cyclosporin A, T-Det LF-416®, and Peanut oil.

Another embodiment comprises cyclosporin A, Tergitol Min-Foam 1X®, and Peanut oil.

Another embodiment comprises cyclosporin A, Tergitol Min-Foam 2X®, and Peanut oil.

Another embodiment comprises cyclosporin A, Triton CF-21®, and Peanut oil.

Another embodiment comprises cyclosporin A, Triton CF-76®, and Peanut oil.

Another embodiment comprises cyclosporin A, Triton XL-80N®, and Peanut oil.

Another embodiment comprises cyclosporin A, Witconol NS-98®, and Peanut oil.

Another embodiment comprises cyclosporin A, Witconol NS-108LQ®, and Peanut oil.

Another embodiment comprises cyclosporin A, Witconol NS-145®, and Peanut oil.

Another embodiment comprises cyclosporin A, Witconol NS-179®, and Peanut oil.

Another embodiment comprises cyclosporin A, a Protein-based Surfactant, and Peanut oil.

Another embodiment comprises cyclosporin A, AminoFoam W®, and Peanut oil.

Another embodiment comprises cyclosporin A, Amiter LGOD-2®, and Peanut oil.

Another embodiment comprises cyclosporin A, Amiter LGS-2®, and Peanut oil.

Another embodiment comprises cyclosporin A, Amiter LGS-5®, and Peanut oil.

Another embodiment comprises cyclosporin A, Lamepon S®, and Peanut oil.

Another embodiment comprises cyclosporin A, Lamepon S®, and Peanut oil.

Another embodiment comprises cyclosporin A, Maypon 4C®, and Peanut oil.

Another embodiment comprises cyclosporin A, May-Tein C®, and Peanut oil.

Another embodiment comprises cyclosporin A, May-Tein CT®, and Peanut oil.

Another embodiment comprises cyclosporin A, May-Tein KTS®, and Peanut oil.

Another embodiment comprises cyclosporin A, May-Tein SY®, and Peanut oil.

Another embodiment comprises cyclosporin A, Plantapon S®, and Peanut oil.

Another embodiment comprises cyclosporin A, Proteol APL®, and Peanut oil.

Another embodiment comprises cyclosporin A, Proteol OAT®, and Peanut oil.

Another embodiment comprises cyclosporin A, Pyroter CPI-40®, and Peanut oil.

Another embodiment comprises cyclosporin A, Pyroter GPI-25®, and Peanut oil.

Another embodiment comprises cyclosporin A, Supro-Tein S®, and Peanut oil.

Another embodiment comprises cyclosporin A, Supro-Tein V®, and Peanut oil.

Another embodiment comprises cyclosporin A,® and Peanut oil.

Another embodiment comprises cyclosporin A, a Sarcosine Derivative®, and Peanut oil.

Another embodiment comprises cyclosporin A, Crodasinic LS-30®, and Peanut oil.

Another embodiment comprises cyclosporin A, Vanseal CS®, and Peanut oil.

Another embodiment comprises cyclosporin A, Vanseal LS®, and Peanut oil.

Another embodiment comprises cyclosporin A, Vanseal MS®, and Peanut oil.

Another embodiment comprises cyclosporin A, Vanseal NACS-30®, and Peanut oil.

Another embodiment comprises cyclosporin A, Vanseal NALS-95®, and Peanut oil.

Another embodiment comprises cyclosporin A, Vanseal OS®, and Peanut oil.

Another embodiment comprises cyclosporin A,® and Peanut oil.

Another embodiment comprises cyclosporin A, a Silicone-based Surfactant®, and Peanut oil.

Another embodiment comprises cyclosporin A, Abil-B-9950®, and Peanut oil.

Another embodiment comprises cyclosporin A, Abil Care 85®, and Peanut oil.

Another embodiment comprises cyclosporin A, Abil EM 90®, and Peanut oil.

Another embodiment comprises cyclosporin A, Abil EM 97®, and Peanut oil.

Another embodiment comprises cyclosporin A, Abil WE-09®, and Peanut oil.

Another embodiment comprises cyclosporin A, Dow Corning 1248 Fluid®, and Peanut oil.

Another embodiment comprises cyclosporin A, Dow Corning 3225C®, and Peanut oil.

Another embodiment comprises cyclosporin A, Dow Corning 5200®, and Peanut oil.

Another embodiment comprises cyclosporin A, Dow Corning Q4-3667®, and Peanut oil.

Another embodiment comprises cyclosporin A, Monasil PCA®, and Peanut oil.

Another embodiment comprises cyclosporin A, Monasil PDM®, and Peanut oil.

Another embodiment comprises cyclosporin A, Monasil PLN®, and Peanut oil.

Another embodiment comprises cyclosporin A, Polyderm PPI-SI-WS®, and Peanut oil.

Another embodiment comprises cyclosporin A, Troysol 380W®, and Peanut oil.

Another embodiment comprises cyclosporin A, Troysol S366®, and Peanut oil.

Another embodiment comprises cyclosporin A, a Sorbitan Derivative, and Peanut oil.

Another embodiment comprises cyclosporin A, Alkamuls SML®, and Peanut oil.

Another embodiment comprises cyclosporin A, Alkamuls SMO®, and Peanut oil.

Another embodiment comprises cyclosporin A, Alkamuls STO®, and Peanut oil.

Another embodiment comprises cyclosporin A, Arlacel 20®, and Peanut oil.

Another embodiment comprises cyclosporin A, Arlacel 40®, and Peanut oil.

Another embodiment comprises cyclosporin A, Arlacel 60®, and Peanut oil.

Another embodiment comprises cyclosporin A, Arlacel 80®, and Peanut oil.

Another embodiment comprises cyclosporin A, Arlacel C®, and Peanut oil.

Another embodiment comprises cyclosporin A, Armul 21®, and Peanut oil.

Another embodiment comprises cyclosporin A, Atlox 80®, and Peanut oil.

Another embodiment comprises cyclosporin A, Atlox 847®, and Peanut oil.

Another embodiment comprises cyclosporin A, Atlox 1045A®, and Peanut oil.

Another embodiment comprises cyclosporin A, Canarcel 20®, and Peanut oil.

Another embodiment comprises cyclosporin A, Canarcel 60®, and Peanut oil.

Another embodiment comprises cyclosporin A, Canarcel 80®, and Peanut oil.

Another embodiment comprises cyclosporin A, Canarcel TW 20®, and Peanut oil.

Another embodiment comprises cyclosporin A, Canarcel TW 60®, and Peanut oil.

Another embodiment comprises cyclosporin A, Canarcel TW 80®, and Peanut oil.

Another embodiment comprises cyclosporin A, Coladet BSB-P®, and Peanut oil.

Another embodiment comprises cyclosporin A, Customulse O-20®, and Peanut oil.

Another embodiment comprises cyclosporin A, Dehymuls E®, and Peanut oil.

Another embodiment comprises cyclosporin A, DeSotan SMO®, and Peanut oil.

Another embodiment comprises cyclosporin A, DeSotan SMO-20®, and Peanut oil.

Another embodiment comprises cyclosporin A, DeSotan SMT®, and Peanut oil.

Another embodiment comprises cyclosporin A, DeSotan SMT-20®, and Peanut oil.

Another embodiment comprises cyclosporin A, Durfax 60®, and Peanut oil.

Another embodiment comprises cyclosporin A, Durfax 65®, and Peanut oil.

Another embodiment comprises cyclosporin A, Durfax 80®, and Peanut oil.

Another embodiment comprises cyclosporin A, Durtan 60®, and Peanut oil.

Another embodiment comprises cyclosporin A, Durtan 65®, and Peanut-oil.

Another embodiment comprises cyclosporin A, Liposorb L®, and Peanut oil.

Another embodiment comprises cyclosporin A, Liposorb L-10®, and Peanut oil.

Another embodiment comprises cyclosporin A, Liposorb L-20®, and Peanut oil.

Another embodiment comprises cyclosporin A, Liposorb O®, and Peanut oil.

Another embodiment comprises cyclosporin A, Liposorb O-20®, and Peanut oil.

Another embodiment comprises cyclosporin A, Liposorb P®, and Peanut oil.

Another embodiment comprises cyclosporin A, Liposorb P-20®, and Peanut oil.

Another embodiment comprises cyclosporin A, Liposorb S®, and Peanut oil.

Another embodiment comprises cyclosporin A, Liposorb S-20®, and Peanut oil.

Another embodiment comprises cyclosporin A, Liposorb SQO®, and Peanut oil.

Another embodiment comprises cyclosporin A, Liposorb TO®, and Peanut oil.

Another embodiment comprises cyclosporin A, Liposorb TS®, and Peanut oil.

Another embodiment comprises cyclosporin A, Liposorb TS-20®, and Peanut oil.

Another embodiment comprises cyclosporin A, Lumisorb PS®, and Peanut oil.

Another embodiment comprises cyclosporin A, Lumisorb SMO (T)®, and Peanut oil.

Another embodiment comprises cyclosporin A, Lumisorb SMS K®, and Peanut oil.

Another embodiment comprises cyclosporin A, Lumisorb SSO®, and Peanut oil.

Another embodiment comprises cyclosporin A, Lumisorb STS K®, and Peanut oil.

Another embodiment comprises cyclosporin A, Lumisorb STT®, and Peanut oil.

Another embodiment comprises cyclosporin A, Magrabar SMO®, and Peanut oil.

Another embodiment comprises cyclosporin A, Magrabar SMO-VEG®, and Peanut oil.

Another embodiment comprises cyclosporin A, Magrabar SMT®, and Peanut oil.

Another embodiment comprises cyclosporin A, Magrabar STO®, and Peanut oil.

Another embodiment comprises cyclosporin A, Miracare BC-27®, and Peanut oil.

Another embodiment comprises cyclosporin A, Ritabate 20®, and Peanut oil.

Another embodiment comprises cyclosporin A, Ritabate 40®, and Peanut oil.

Another embodiment comprises cyclosporin A, Ritabate 60®, and Peanut oil.

Another embodiment comprises cyclosporin A, Ritabate 80®, and Peanut oil.

Another embodiment comprises cyclosporin A, T-Maz®, and Peanut oil.

Another embodiment comprises cyclosporin A, Tego SML®, and Peanut oil.

Another embodiment comprises cyclosporin A, Tego SML 20®, and Peanut oil.

Another embodiment comprises cyclosporin A, Tego SMO 80 V®, and Peanut oil.

Another embodiment comprises cyclosporin A, Tego SMO V®, and Peanut oil.

Another embodiment comprises cyclosporin A, Tego SMS®, and Peanut oil.

Another embodiment comprises cyclosporin A, Tego STO V®, and Peanut oil.

Another embodiment comprises cyclosporin A, Tween 21®, and Peanut oil.

Another embodiment comprises cyclosporin A, Tween 40®, and Peanut oil.

Another embodiment comprises cyclosporin A, Tween 60®, and Peanut oil.

Another embodiment comprises cyclosporin A, Tween 60 K®, and Peanut oil.

Another embodiment comprises cyclosporin A, Tween 61®, and Peanut oil.

Another embodiment comprises cyclosporin A, Tween 65®, and Peanut oil.

Another embodiment comprises cyclosporin A, Tween 80®, and Peanut oil.

Another embodiment comprises cyclosporin A, Tween 80 K®, and Peanut oil.

Another embodiment comprises cyclosporin A, Tween 81®, and Peanut oil.

Another embodiment comprises cyclosporin A, Tween 85®, and Peanut oil.

Another embodiment comprises cyclosporin A, a Sucrose or Glucose Ester, or Derivative thereof, and Peanut oil.

Another embodiment comprises cyclosporin A, DeSulf GOS-P-60WCG®, and Peanut oil.

Another embodiment comprises cyclosporin A, Glucam E-20 Distearate®, and Peanut oil.

Another embodiment comprises cyclosporin A, Glucamate DOE-120®, and Peanut oil.

Another embodiment comprises cyclosporin A, Glucamate SSE-20®, and Peanut oil.

Another embodiment comprises cyclosporin A, Glucate DO®, and Peanut oil.

Another embodiment comprises cyclosporin A, Glucate SS®, and Peanut oil.

Another embodiment comprises cyclosporin A, Glucopon 425 UP®, and Peanut oil.

Another embodiment comprises cyclosporin A, Isolan IS®, and Peanut oil.

Another embodiment comprises cyclosporin A, Mazon 40®, and Peanut oil.

Another embodiment comprises cyclosporin A, Montanov 82®, and Peanut oil.

Another embodiment comprises cyclosporin A, Montanov 202®, and Peanut oil.

Another embodiment comprises cyclosporin A, Montanov S®, and Peanut oil.

Another embodiment comprises cyclosporin A, Rheozan®, and Peanut oil.

Another embodiment comprises cyclosporin A, Simulsol AS 48®, and Peanut oil.

Another embodiment comprises cyclosporin A, Simulsol SL 4®, and Peanut oil.

Another embodiment comprises cyclosporin A, Simulsol SL 10®, and Peanut oil.

Another embodiment comprises cyclosporin A, Simulsol SL 11W®, and Peanut oil.

Another embodiment comprises cyclosporin A, Simulsol SL 55®, and Peanut oil.

Another embodiment comprises cyclosporin A, Suga Nate 100 and 160®, and Peanut oil.

Another embodiment comprises cyclosporin A, Tego Care 450®, and Peanut oil.

Another embodiment comprises cyclosporin A, Tego Care CG 90®, and Peanut oil.

Another embodiment comprises cyclosporin A, Tego Care PS®, and Peanut oil.

Another embodiment comprises cyclosporin A, Tegosoft PSE 141 G®, and Peanut oil.

Another embodiment comprises cyclosporin A, Tegotens G 826®, and Peanut oil.

Another embodiment comprises cyclosporin A, Triton BG-10 (70%)®, and Peanut oil.

Another embodiment comprises cyclosporin A, Triton CG-110 (60%)®, and Peanut oil.

Another embodiment comprises cyclosporin A, Wickenol 545®, and Peanut oil.

Another embodiment comprises cyclosporin A, an alcohol, and Peppermint oil.

Another embodiment comprises cyclosporin A, Diglycerol®, and Peppermint oil.

Another embodiment comprises cyclosporin A, Hetoxide GT-80®, and Peppermint oil.

Another embodiment comprises cyclosporin A, Lexemul BEO®, and Peppermint oil.

Another embodiment comprises cyclosporin A, Polyglycerol-3®, and Peppermint oil.

Another embodiment comprises cyclosporin A, Redicote E Series®, and Peppermint oil.

Another embodiment comprises cyclosporin A, Simulsol OX 1005L®, and Peppermint oil.

Another embodiment comprises cyclosporin A, Stanfax 567®, and Peppermint oil.

Another embodiment comprises cyclosporin A, TA-1618®, and Peppermint oil.

Another embodiment comprises cyclosporin A, Witconol H-31A®, and Peppermint oil.

Another embodiment comprises cyclosporin A, an amine oxide, and Peppermint oil.

Another embodiment comprises cyclosporin A, AO-405®, and Peppermint oil.

Another embodiment comprises cyclosporin A, AO-455®, and Peppermint oil.

Another embodiment comprises cyclosporin A, AO 728 Special®, and Peppermint oil.

Another embodiment comprises cyclosporin A, Barlox 12®, and Peppermint oil.

Another embodiment comprises cyclosporin A, Barlox 14®, and Peppermint oil.

Another embodiment comprises cyclosporin A, Burcoxide Lo®, and Peppermint oil.

Another embodiment comprises cyclosporin A, Caloxamine LO®, and Peppermint oil.

Another embodiment comprises cyclosporin A, Chemoxide CAW®, and Peppermint oil.

Another embodiment comprises cyclosporin A, Chemoxide LM-30®, and Peppermint oil.

Another embodiment comprises cyclosporin A, Chemoxide LO®, and Peppermint oil.

Another embodiment comprises cyclosporin A, Chemoxide MO®, and Peppermint oil.

Another embodiment comprises cyclosporin A, Colalux CAO-35®, and Peppermint oil.

Another embodiment comprises cyclosporin A, Colalux LO®, and Peppermint oil.

Another embodiment comprises cyclosporin A, DeMox CAPO®, and Peppermint oil.

Another embodiment comprises cyclosporin A, DeMox CSG-30®, and Peppermint oil.

Another embodiment comprises cyclosporin A, DeMox LAO®, and Peppermint oil.

Another embodiment comprises cyclosporin A, Emcol L®, and Peppermint oil.

Another embodiment comprises cyclosporin A, Empigen OB®, and Peppermint oil.

Another embodiment comprises cyclosporin A, Empigen OS/A®, and Peppermint oil.

Another embodiment comprises cyclosporin A, Foamox CDO®, and Peppermint oil.

Another embodiment comprises cyclosporin A, Foamox DMM®, and Peppermint oil.

Another embodiment comprises cyclosporin A, Foamox DMS®, and Peppermint oil.

Another embodiment comprises cyclosporin A, Genaminox KC®, and Peppermint oil.

Another embodiment comprises cyclosporin A, Genaminox LA®, and Peppermint oil.

Another embodiment comprises cyclosporin A, Hartofoam SAO®, and Peppermint oil.

Another embodiment comprises cyclosporin A, Hartox DMCD®, and Peppermint oil.

Another embodiment comprises cyclosporin A, Lipowax DAT®, and Peppermint oil.

Another embodiment comprises cyclosporin A, Lipowax PB Pastilles®, and Peppermint oil.

Another embodiment comprises cyclosporin A, Mackamine C8®, and Peppermint oil.

Another embodiment comprises cyclosporin A, Mackamine C10®, and Peppermint oil.

Another embodiment comprises cyclosporin A, Mackamine C 14®, and Peppermint oil.

Another embodiment comprises cyclosporin A, Mackamine CAO®, and Peppermint oil.

Another embodiment comprises cyclosporin A, Mackamine CO®, and Peppermint oil.

Another embodiment comprises cyclosporin A, Mackamine LO®, and Peppermint oil.

Another embodiment comprises cyclosporin A, Mackamine O2®, and Peppermint oil.

Another embodiment comprises cyclosporin A, Mackamine SAO®, and Peppermint oil.

Another embodiment comprises cyclosporin A, Mackamine SO®, and Peppermint oil.

Another embodiment comprises cyclosporin A, Mazox KCAO®, and Peppermint oil.

Another embodiment comprises cyclosporin A, Monalac MO®, and Peppermint oil.

Another embodiment comprises cyclosporin A, Norfox LDA®, and Peppermint oil.

Another embodiment comprises cyclosporin A, Rhodamox LO®, and Peppermint oil.

Another embodiment comprises cyclosporin A, Schercamox C-AA®, and Peppermint oil.

Another embodiment comprises cyclosporin A, Schercamox DMA®, and Peppermint oil.

Another embodiment comprises cyclosporin A, Schercamox DML®, and Peppermint oil.

Another embodiment comprises cyclosporin A, Schercamox DMM®, and Peppermint oil.

Another embodiment comprises cyclosporin A, Schercamox DMS®, and Peppermint oil.

Another embodiment comprises cyclosporin A, Tegotens DO®, and Peppermint oil.

Another embodiment comprises cyclosporin A, Tomah AO-14-2®, and Peppermint oil.

Another embodiment comprises cyclosporin A, Triaminox CDO®, and Peppermint oil.

Another embodiment comprises cyclosporin A, a block polymer, and Peppermint oil.

Another embodiment comprises cyclosporin A, AL 2070®, and Peppermint oil.

Another embodiment comprises cyclosporin A, Antarox 17-R-2®, and Peppermint oil.

Another embodiment comprises cyclosporin A, Antarox 25-R-2®, and Peppermint oil.

Another embodiment comprises cyclosporin A, Antarox 31 -R-1®, and Peppermint oil.

Another embodiment comprises cyclosporin A, Antarox P-84®, and Peppermint oil.

Another embodiment comprises cyclosporin A, Antarox P-104/H®, and Peppermint oil.

Another embodiment comprises cyclosporin A, Arnox BP-Series®, and Peppermint oil.

Another embodiment comprises cyclosporin A, Chemonic 435®, and Peppermint oil.

Another embodiment comprises cyclosporin A, Chemonic D-25®, and Peppermint oil.

Another embodiment comprises cyclosporin A, Chemonic PL Series®, and Peppermint oil.

Another embodiment comprises cyclosporin A, Ethox L-121®, and Peppermint oil.

Another embodiment comprises cyclosporin A, Ethox L-122®, and Peppermint oil.

Another embodiment comprises cyclosporin A, Genapol PF-10®, and Peppermint oil.

Another embodiment comprises cyclosporin A, Genapol PF-20®, and Peppermint oil.

Another embodiment comprises cyclosporin A, Genapol PF-40A®, and Peppermint oil.

Another embodiment comprises cyclosporin A, Norfox 2-LF®, and Peppermint oil.

Another embodiment comprises cyclosporin A, Simulsol NW 342®, and Peppermint oil.

Another embodiment comprises cyclosporin A, T-Det BP-1®, and Peppermint oil.

Another embodiment comprises cyclosporin A, T-Det XD®, and Peppermint oil.

Another embodiment comprises cyclosporin A, T-Det XH®, and Peppermint oil.

Another embodiment comprises cyclosporin A, Triton CF-32®, and Peppermint oil.

Another embodiment comprises cyclosporin A, Witconol 171®, and Peppermint oil.

Another embodiment comprises cyclosporin A, Witconol 324®, and Peppermint oil.

Another embodiment comprises cyclosporin A, Witconol 324D®, and Peppermint oil.

Another embodiment comprises cyclosporin A, Witconol PD-2000®, and Peppermint oil.

Another embodiment comprises cyclosporin A, a Carboxylated Alcohol or Alkylphenol Ethoxylate, and Peppermint oil.

Another embodiment comprises cyclosporin A, Emcol CN-6®, and Peppermint oil.

Another embodiment comprises cyclosporin A, Ethcarb®, and Peppermint oil.

Another embodiment comprises cyclosporin A, Gemtex WNT-Conc®, and Peppermint oil.

Another embodiment comprises cyclosporin A, Incrodet TD7-C®, and Peppermint oil.

Another embodiment comprises cyclosporin A, Marlinat CM 105/80®, and Peppermint oil.

Another embodiment comprises cyclosporin A, Marlowet 1072®, and Peppermint oil.

Another embodiment comprises cyclosporin A, Marlowet 4530®, and Peppermint oil.

Another embodiment comprises cyclosporin A, Marlowet 4530 LF®, and Peppermint oil.

Another embodiment comprises cyclosporin A, Marlowet 4534®, and Peppermint oil.

Another embodiment comprises cyclosporin A, Marlowet 4538®, and Peppermint oil.

Another embodiment comprises cyclosporin A, Marlowet 4539®, and Peppermint oil.

Another embodiment comprises cyclosporin A, Marlowet 4539 LF®, and Peppermint oil.

Another embodiment comprises cyclosporin A, Marlowet 4541®, and Peppermint oil.

Another embodiment comprises cyclosporin A, Miranate LEC-80®, and Peppermint oil.

Another embodiment comprises cyclosporin A, Sandopan B®, and Peppermint oil.

Another embodiment comprises cyclosporin A, Sandopan B Modified®, and Peppermint oil.

Another embodiment comprises cyclosporin A, Sandopan LS-24 Gel®, and Peppermint oil.

Another embodiment comprises cyclosporin A, Surfine T-A®, and Peppermint oil.

Another embodiment comprises cyclosporin A, a Carboxylic Acid/Fatty Acid, and Peppermint oil.

Another embodiment comprises cyclosporin A, Colaterge RAM®, and Peppermint oil.

Another embodiment comprises cyclosporin A, Colatrope INC®, and Peppermint oil.

Another embodiment comprises cyclosporin A, Crodacid B®, and Peppermint oil.

Another embodiment comprises cyclosporin A, DeTrope CA-100®, and Peppermint oil.

Another embodiment comprises cyclosporin A, Latol MTO®, and Peppermint oil.

Another embodiment comprises cyclosporin A, Lumulse CC-33 K®, and Peppermint oil.

Another embodiment comprises cyclosporin A, Mulls 2218®, and Peppermint oil.

Another embodiment comprises cyclosporin A, OL-600®, and Peppermint oil.

Another embodiment comprises cyclosporin A, OL-800®, and Peppermint oil.

Another embodiment comprises cyclosporin A, R-910®, and Peppermint oil.

Another embodiment comprises cyclosporin A, S-210®, and Peppermint oil.

Another embodiment comprises cyclosporin A, Sandopan DTC Acid®, and Peppermint oil.

Another embodiment comprises cyclosporin A, Sandopan LS 24 N®, and Peppermint oil.

Another embodiment comprises cyclosporin A, Sandopan MA-18®, and Peppermint oil.

Another embodiment comprises cyclosporin A, an Ethoxylated Alcohol, and Peppermint oil.

Another embodiment comprises cyclosporin A, Adsee 799®, and Peppermint oil.

Another embodiment comprises cyclosporin A, Adsee 799®, and Peppermint oil.

Another embodiment comprises cyclosporin A, Alfonic 610-3.5®, and Peppermint oil.

Another embodiment comprises cyclosporin A, Alfonic 810-2®, and Peppermint oil.

Another embodiment comprises cyclosporin A, Alfonic 810-6®, and Peppermint oil.

Another embodiment comprises cyclosporin A, Alfonic 1012-3®, and Peppermint oil.

Another embodiment comprises cyclosporin A, Alfonic 1012-5®, and Peppermint oil.

Another embodiment comprises cyclosporin A, Alfonic 1216CO-1.5®, and Peppermint oil.

Another embodiment comprises cyclosporin A, Alfonic 1216CO-7®, and Peppermint oil.

Another embodiment comprises cyclosporin A, Alfonic 1412-3®, and Peppermint oil.

Another embodiment comprises cyclosporin A, Alfonic 1412-7®, and Peppermint oil.

Another embodiment comprises cyclosporin A, Arlasolve 200®, and Peppermint oil.

Another embodiment comprises cyclosporin A, Arlasolve 200 Liquid®, and Peppermint oil.

Another embodiment comprises cyclosporin A, Armix 180-C®, and Peppermint oil.

Another embodiment comprises cyclosporin A, Armix 183®, and Peppermint oil.

Another embodiment comprises cyclosporin A, Armul 2404®, and Peppermint oil.

Another embodiment comprises cyclosporin A, Armul 2404®, and Peppermint oil.

Another embodiment comprises cyclosporin A, Atlas EMJ-C®, and Peppermint oil.

Another embodiment comprises cyclosporin A, Atlas G-2109®, and Peppermint oil.

Another embodiment comprises cyclosporin A, Atlas G-3886®, and Peppermint oil.

Another embodiment comprises cyclosporin A, Atlas G-3890®, and Peppermint oil.

Another embodiment comprises cyclosporin A, Bio Soft E-200®, and Peppermint oil.

Another embodiment comprises cyclosporin A, Bio Soft E-300®, and Peppermint oil.

Another embodiment comprises cyclosporin A, Bio Soft E-400®, and Peppermint oil.

Another embodiment comprises cyclosporin A, Bio Soft EN 600®, and Peppermint oil.

Another embodiment comprises cyclosporin A, Bio Soft TD-400®, and Peppermint oil.

Another embodiment comprises cyclosporin A, Bio Soft TD-630®, and Peppermint oil.

Another embodiment comprises cyclosporin A, Brij 30®, and Peppermint oil.

Another embodiment comprises cyclosporin A, Brij 52®, and Peppermint oil.

Another embodiment comprises cyclosporin A, Brij 56®, and Peppermint oil.

Another embodiment comprises cyclosporin A, Brij 58®, and Peppermint oil.

Another embodiment comprises cyclosporin A, Brij 72®, and Peppermint oil.

Another embodiment comprises cyclosporin A, Brij 76®, and Peppermint oil.

Another embodiment comprises cyclosporin A, Brij 78®, and Peppermint oil.

Another embodiment comprises cyclosporin A, Brij 93®, and Peppermint oil.

Another embodiment comprises cyclosporin A, Brij 97®, and Peppermint oil.

Another embodiment comprises cyclosporin A, Brij 98®, and Peppermint oil.

Another embodiment comprises cyclosporin A, Brij 700®, and Peppermint oil.

Another embodiment comprises cyclosporin A, Brij 700 S®, and Peppermint oil.

Another embodiment comprises cyclosporin A, Brij 721®, and Peppermint oil.

Another embodiment comprises cyclosporin A, Brij 721 S®, and Peppermint oil.

Another embodiment comprises cyclosporin A, Burcoterge CDG®, and Peppermint oil.

Another embodiment comprises cyclosporin A, Canasol AT 600®, and Peppermint oil.

Another embodiment comprises cyclosporin A, Canasol AT 800®, and Peppermint oil.

Another embodiment comprises cyclosporin A, Canasol AT 1200®, and Peppermint oil.

Another embodiment comprises cyclosporin A, Canasol BJ 35®, and Peppermint oil.

Another embodiment comprises cyclosporin A, Canasol BJ 36®, and Peppermint oil.

Another embodiment comprises cyclosporin A, Canasol BJ 52®, and Peppermint oil.

Another embodiment comprises cyclosporin A, Canasol BJ 58®, and Peppermint oil.

Another embodiment comprises cyclosporin A, Canasol BJ 72®, and Peppermint oil.

Another embodiment comprises cyclosporin A, Canasol BJ 78®, and Peppermint oil.

Another embodiment comprises cyclosporin A, Canasol BJ 98®, and Peppermint oil.

Another embodiment comprises cyclosporin A, Canasol BJ 307®, and Peppermint oil.

Another embodiment comprises cyclosporin A, Cerfak 1400®, and Peppermint oil.

Another embodiment comprises cyclosporin A, Cetomacrogol 1000 BP®, and Peppermint oil.

Another embodiment comprises cyclosporin A, Chemonic C-2®, and Peppermint oil.

Another embodiment comprises cyclosporin A, Chemonic C-10®, and Peppermint oil.

Another embodiment comprises cyclosporin A, Chemonic C-20®, and Peppermint oil.

Another embodiment comprises cyclosporin A, Chemonic CT-12®, and Peppermint oil.

Another embodiment comprises cyclosporin A, Chemonic CT-20®, and Peppermint oil.

Another embodiment comprises cyclosporin A, Chemonic CT-30®, and Peppermint oil.

Another embodiment comprises cyclosporin A, Chemonic CT-55®, and Peppermint oil.

Another embodiment comprises cyclosporin A, Chemonic G-7®, and Peppermint oil.

Another embodiment comprises cyclosporin A, Chemonic G-26®, and Peppermint oil.

Another embodiment comprises cyclosporin A, Chemonic L-4®, and Peppermint oil.

Another embodiment comprises cyclosporin A, Chemonic L-7®, and Peppermint oil.

Another embodiment comprises cyclosporin A, Chemonic L-12®, and Peppermint oil.

Another embodiment comprises cyclosporin A, Chemonic L-23®, and Peppermint oil.

Another embodiment comprises cyclosporin A, Chemonic O-2®, and Peppermint oil.

Another embodiment comprises cyclosporin A, Chemonic O-5®, and Peppermint oil.

Another embodiment comprises cyclosporin A, Chemonic O-10®, and Peppermint oil.

Another embodiment comprises cyclosporin A, Chemonic O-20®, and Peppermint oil.

Another embodiment comprises cyclosporin A, Chemonic S-2®, and Peppermint oil.

Another embodiment comprises cyclosporin A, Chemonic S-10®, and Peppermint oil.

Another embodiment comprises cyclosporin A, Chemonic S-20®, and Peppermint oil.

Another embodiment comprises cyclosporin A, Colamulse FE®, and Peppermint oil.

Another embodiment comprises cyclosporin A, Cremophor A 20®, and Peppermint oil.

Another embodiment comprises cyclosporin A, Cremophor SA 2®, and Peppermint oil.

Another embodiment comprises cyclosporin A, Dehydol 100®, and Peppermint oil.

Another embodiment comprises cyclosporin A, Dehydol O-4®, and Peppermint oil.

Another embodiment comprises cyclosporin A, Delonic C-18®, and Peppermint oil.

Another embodiment comprises cyclosporin A, DeSonic 6T®, and Peppermint oil.

Another embodiment comprises cyclosporin A, DeSonic 9D®, and Peppermint oil.

Another embodiment comprises cyclosporin A, DeSonic 9T®, and Peppermint oil.

Another embodiment comprises cyclosporin A, DeSonic 12D®, and Peppermint oil.

Another embodiment comprises cyclosporin A, DeSonic 12T®, and Peppermint oil.

Another embodiment comprises cyclosporin A, DeSonic 15T®, and Peppermint oil.

Another embodiment comprises cyclosporin A, DeSonic TDA-9®, and Peppermint oil.

Another embodiment comprises cyclosporin A, DeThox GLG-7®, and Peppermint oil.

Another embodiment comprises cyclosporin A, DeThox GLG-26®, and Peppermint oil.

Another embodiment comprises cyclosporin A, DeThox LA-4®, and Peppermint oil.

Another embodiment comprises cyclosporin A, DeThox LA-23®, and Peppermint oil.

Another embodiment comprises cyclosporin A, DeThox SA-80®, and Peppermint oil.

Another embodiment comprises cyclosporin A, Disponil O5®, and Peppermint oil.

Another embodiment comprises cyclosporin A, Eccoterge EO-41B®, and Peppermint oil.

Another embodiment comprises cyclosporin A, Empilan KA2.5/90®, and Peppermint oil.

Another embodiment comprises cyclosporin A, Empilan KA5/90®, and Peppermint oil.

Another embodiment comprises cyclosporin A, Empilan KM-20®, and Peppermint oil.

Another embodiment comprises cyclosporin A, Empilan KM-50®, and Peppermint oil.

Another embodiment comprises cyclosporin A, Empilan L-23®, and Peppermint oil.

Another embodiment comprises cyclosporin A, Ethylan 25-3®, and Peppermint oil.

Another embodiment comprises cyclosporin A, Ethylan 1204®, and Peppermint oil.

Another embodiment comprises cyclosporin A, Ethylan DA-4®, and Peppermint oil.

Another embodiment comprises cyclosporin A, Ethylan LA-230®, and Peppermint oil.

Another embodiment comprises cyclosporin A, Ethylan SN®, and Peppermint oil.

Another embodiment comprises cyclosporin A, Ethylan TD-60®, and Peppermint oil.

Another embodiment comprises cyclosporin A, Ethylan TD-100®, and Peppermint oil.

Another embodiment comprises cyclosporin A, Ethylan TD-1407®, and Peppermint oil.

Another embodiment comprises cyclosporin A, Eumulgin B1®, and Peppermint oil.

Another embodiment comprises cyclosporin A, Eumulgin B2®, and Peppermint oil.

Another embodiment comprises cyclosporin A, Eumulgin B3®, and Peppermint oil.

Another embodiment comprises cyclosporin A, Eumulgin O-10®, and Peppermint oil.

Another embodiment comprises cyclosporin A, Flo Mo 80/20®, and Peppermint oil.

Another embodiment comprises cyclosporin A, Flo Mo Low Foam®, and Peppermint oil.

Another embodiment comprises cyclosporin A, Forlan C-24®, and Peppermint oil.

Another embodiment comprises cyclosporin A, Genapol 1454®, and Peppermint oil.

Another embodiment comprises cyclosporin A, Genapol BA-020®, and Peppermint oil.

Another embodiment comprises cyclosporin A, Genapol BA-040®, and Peppermint oil.

Another embodiment comprises cyclosporin A, Genapol C-100®, and Peppermint oil.

Another embodiment comprises cyclosporin A, Genapol DA 060®, and Peppermint oil.

Another embodiment comprises cyclosporin A, Genapol HS 020®, and Peppermint oil.

Another embodiment comprises cyclosporin A, Genapol HS 200®, and Peppermint oil.

Another embodiment comprises cyclosporin A, Genapol ID-040®, and Peppermint oil.

Another embodiment comprises cyclosporin A, Genapol ID-060®, and Peppermint oil.

Another embodiment comprises cyclosporin A, Genapol ID-090®, and Peppermint oil.

Another embodiment comprises cyclosporin A, Genapol LA 010®, and Peppermint oil.

Another embodiment comprises cyclosporin A, Genapol LA 020®, and Peppermint oil.

Another embodiment comprises cyclosporin A, Genapol LA 030®, and Peppermint oil.

Another embodiment comprises cyclosporin A, Genapol LA 040®, and Peppermint oil.

Another embodiment comprises cyclosporin A, Genapol LA 050®, and Peppermint oil.

Another embodiment comprises cyclosporin A, Genapol LA 060®, and Peppermint oil.

Another embodiment comprises cyclosporin A, Genapol LA 070®, and Peppermint oil.

Another embodiment comprises cyclosporin A, Genapol LA 070S®, and Peppermint oil.

Another embodiment comprises cyclosporin A, Genapol LA 230®, and Peppermint oil.

Another embodiment comprises cyclosporin A, Genapol O 020®, and Peppermint oil.

Another embodiment comprises cyclosporin A, Genapol O 050®, and Peppermint oil.

Another embodiment comprises cyclosporin A, Genapol O 100®, and Peppermint oil.

Another embodiment comprises cyclosporin A, Genapol O 200®, and Peppermint oil.

Another embodiment comprises cyclosporin A, Genapol SA 030®, and Peppermint oil.

Another embodiment comprises cyclosporin A, Genapol SA 120®, and Peppermint oil.

Another embodiment comprises cyclosporin A, Genapol T-020®, and Peppermint oil.

Another embodiment comprises cyclosporin A, Genapol UD-030®, and Peppermint oil.

Another embodiment comprises cyclosporin A, Genapol UD-050®, and Peppermint oil.

Another embodiment comprises cyclosporin A, Genapol UD-070®, and Peppermint oil.

Another embodiment comprises cyclosporin A, Genapol UD-079®, and Peppermint oil.

Another embodiment comprises cyclosporin A, Genapol UD-080®, and Peppermint oil.

Another embodiment comprises cyclosporin A, Genapol UD-110®, and Peppermint oil.

Another embodiment comprises cyclosporin A, Genapol X 030®, and Peppermint oil.

Another embodiment comprises cyclosporin A, Genapol X 050®, and Peppermint oil.

Another embodiment comprises cyclosporin A, Genapol X 060®, and Peppermint oil.

Another embodiment comprises cyclosporin A, Genapol X 070®, and Peppermint oil.

Another embodiment comprises cyclosporin A, Genapol X 080®, and Peppermint oil.

Another embodiment comprises cyclosporin A, Genapol X 100®, and Peppermint oil.

Another embodiment comprises cyclosporin A, Genapol X159®, and Peppermint oil.

Another embodiment comprises cyclosporin A, Generol 122 E5®, and Peppermint oil.

Another embodiment comprises cyclosporin A, Generol 122 E25®, and Peppermint oil.

Another embodiment comprises cyclosporin A, Hostacerin T-3®, and Peppermint oil.

Another embodiment comprises cyclosporin A, Iconol LF 110®, and Peppermint oil.

Another embodiment comprises cyclosporin A, Incropol CS-20®, and Peppermint oil.

Another embodiment comprises cyclosporin A, Lexemul CS-20®, and Peppermint oil.

Another embodiment comprises cyclosporin A, Liponic EG-1®, and Peppermint oil.

Another embodiment comprises cyclosporin A, Lipowax D®, and Peppermint oil.

Another embodiment comprises cyclosporin A, Lipowax G®, and Peppermint oil.

Another embodiment comprises cyclosporin A, Lipowax NI®, and Peppermint oil.

Another embodiment comprises cyclosporin A, Lipowax P®, and Peppermint oil.

Another embodiment comprises cyclosporin A, Lipowax P-31®, and Peppermint oil.

Another embodiment comprises cyclosporin A, Lipowax PR®, and Peppermint oil.

Another embodiment comprises cyclosporin A, Lumulse CS-20®, and Peppermint oil.

Another embodiment comprises cyclosporin A, Macol CSA-20®, and Peppermint oil.

Another embodiment comprises cyclosporin A, Marlox B 24/50®, and Peppermint oil.

Another embodiment comprises cyclosporin A, Mazawet 77®, and Peppermint oil.

Another embodiment comprises cyclosporin A, Norfox 1713®, and Peppermint oil.

Another embodiment comprises cyclosporin A, Norfox 2579®, and Peppermint oil.

Another embodiment comprises cyclosporin A, Norfox Lo Foam®, and Peppermint oil.

Another embodiment comprises cyclosporin A, Promulgen D®, and Peppermint oil.

Another embodiment comprises cyclosporin A, Promulgen G®, and Peppermint oil.

Another embodiment comprises cyclosporin A, Renex 30®, and Peppermint oil.

Another embodiment comprises cyclosporin A, Renex 36®, and Peppermint oil.

Another embodiment comprises cyclosporin A, Rhodasurf A 24®, and Peppermint oil.

Another embodiment comprises cyclosporin A, Rhodasurf AAE-10®, and Peppermint oil.

Another embodiment comprises cyclosporin A, Rhodasurf BEH-25®, and Peppermint oil.

Another embodiment comprises cyclosporin A, Rhodasurf BEH-40®, and Peppermint oil.

Another embodiment comprises cyclosporin A, Rhodasurf DA 530®, and Peppermint oil.

Another embodiment comprises cyclosporin A, Rhodasurf DA 630®, and Peppermint oil.

Another embodiment comprises cyclosporin A, Rhodasun DA 639®, and Peppermint oil.

Another embodiment comprises cyclosporin A, Rhodasurf LAN-23®, and Peppermint oil.

Another embodiment comprises cyclosporin A, Rhodasurf ON-870®, and Peppermint oil.

Another embodiment comprises cyclosporin A, Rhodasurf ON-877®, and Peppermint oil.

Another embodiment comprises cyclosporin A, Rhodasurf TB-970 FLK®, and Peppermint oil.

Another embodiment comprises cyclosporin A, Ritacet-20®, and Peppermint oil.

Another embodiment comprises cyclosporin A, Ritachol 1000®, and Peppermint oil.

Another embodiment comprises cyclosporin A, Ritachol 2000®, and Peppermint oil.

Another embodiment comprises cyclosporin A, Ritachol 5000®, and Peppermint oil.

Another embodiment comprises cyclosporin A, Ritox 35®, and Peppermint oil.

Another embodiment comprises cyclosporin A, Surtonic DA-4®, and Peppermint oil.

Another embodiment comprises cyclosporin A, Surtonic DA-6®, and Peppermint oil.

Another embodiment comprises cyclosporin A, Surfonic L46-7®, and Peppermint oil.

Another embodiment comprises cyclosporin A, Surfonic POA®, and Peppermint oil.

Another embodiment comprises cyclosporin A, Synthrapol KB®, and Peppermint oil.

Another embodiment comprises cyclosporin A, Teginacid®, and Peppermint oil.

Another embodiment comprises cyclosporin A, Teginacid C®, and Peppermint oil.

Another embodiment comprises cyclosporin A, Tegotens EC 11®, and Peppermint oil.

Another embodiment comprises cyclosporin A, Tinegal NA®, and Peppermint oil.

Another embodiment comprises cyclosporin A, Tomadol 400®, and Peppermint oil.

Another embodiment comprises cyclosporin A, Tomadol 600®, and Peppermint oil.

Another embodiment comprises cyclosporin A, Tomadol 900®, and Peppermint oil.

Another embodiment comprises cyclosporin A, Uniperol O®, and Peppermint oil.

Another embodiment comprises cyclosporin A, Witconol SN Series®, and Peppermint oil.

Another embodiment comprises cyclosporin A, an Ethoxylated Alkylphenol, and Peppermint oil.

Another embodiment comprises cyclosporin A, Antarox LF-222®, and Peppermint oil.

Another embodiment comprises cyclosporin A, Atlox 775®, and Peppermint oil.

Another embodiment comprises cyclosporin A, Caloxylate N-9®, and Peppermint oil.

Another embodiment comprises cyclosporin A, Canasol NF-1000®, and Peppermint oil.

Another embodiment comprises cyclosporin A, Canasol NF-3000®, and Peppermint oil.

Another embodiment comprises cyclosporin A, Canasol NF-3070®, and Peppermint oil.

Another embodiment comprises cyclosporin A, Canasol OF 1670®, and Peppermint oil.

Another embodiment comprises cyclosporin A, Canasol OF 2570®, and Peppermint oil.

Another embodiment comprises cyclosporin A, Canasol OF 4070®, and Peppermint oil.

Another embodiment comprises cyclosporin A, Chemax DNP-8®, and Peppermint oil.

Another embodiment comprises cyclosporin A, Chemax DNP-18®, and Peppermint oil.

Another embodiment comprises cyclosporin A, Chemax DNP-150/50®, and Peppermint oil.

Another embodiment comprises cyclosporin A, DeSonic 1.5N®, and Peppermint oil.

Another embodiment comprises cyclosporin A, DeSonic 4N®, and Peppermint oil.

Another embodiment comprises cyclosporin A, DeSonic 5N®, and Peppermint oil.

Another embodiment comprises cyclosporin A, DeSonic 6D®, and Peppermint oil.

Another embodiment comprises cyclosporin A, DeSonic 6N®, and Peppermint oil.

Another embodiment comprises cyclosporin A, DeSonic 7N®, and Peppermint oil.

Another embodiment comprises cyclosporin A, DeSonic 9N®, and Peppermint oil.

Another embodiment comprises cyclosporin A, DeSonic 10D®, and Peppermint oil.

Another embodiment comprises cyclosporin A, DeSonic 11N®, and Peppermint oil.

Another embodiment comprises cyclosporin A, DeSonic 12N®, and Peppermint oil.

Another embodiment comprises cyclosporin A, DeSonic 15N®, and Peppermint oil.

Another embodiment comprises cyclosporin A, DeSonic 20N®, and Peppermint oil.

Another embodiment comprises cyclosporin A, Eccoscour RC®, and Peppermint oil.

Another embodiment comprises cyclosporin A, Eccoterge EO-100®, and Peppermint oil.

Another embodiment comprises cyclosporin A, Emulsifier 632/90%®, and Peppermint oil.

Another embodiment comprises cyclosporin A, Geronol AG-821®, and Peppermint oil.

Another embodiment comprises cyclosporin A, Gradonic N-95®, and Peppermint oil.

Another embodiment comprises cyclosporin A, Hetoxide NP-4®, and Peppermint oil.

Another embodiment comprises cyclosporin A, Hetoxide NP-30®, and Peppermint oil.

Another embodiment comprises cyclosporin A, Hostapal N-100®, and Peppermint oil.

Another embodiment comprises cyclosporin A, Hostapal N-110®, and Peppermint oil.

Another embodiment comprises cyclosporin A, Igepal CTA-639W®, and Peppermint oil.

Another embodiment comprises cyclosporin A, Igepal DAP-9®, and Peppermint oil.

Another embodiment comprises cyclosporin A, Igepal OD-410®, and Peppermint oil.

Another embodiment comprises cyclosporin A, Igepal SS-837®, and Peppermint oil.

Another embodiment comprises cyclosporin A, Lipocol NP-9 USP®, and Peppermint oil.

Another embodiment comprises cyclosporin A, Macol DNP-10®, and Peppermint oil.

Another embodiment comprises cyclosporin A, Marlophen NP 5®, and Peppermint oil.

Another embodiment comprises cyclosporin A, Marlophen P 1®, and Peppermint oil.

Another embodiment comprises cyclosporin A, Surfonic NB®, and Peppermint oil.

Another embodiment comprises cyclosporin A, Surfonic OPB-307®, and Peppermint oil.

Another embodiment comprises cyclosporin A, Surfonic OPB-407®, and Peppermint oil.

Another embodiment comprises cyclosporin A, Syn Fac 334®, and Peppermint oil.

Another embodiment comprises cyclosporin A, Syn Fac 8216®, and Peppermint oil.

Another embodiment comprises cyclosporin A, Triton N-57®, and Peppermint oil.

Another embodiment comprises cyclosporin A, Trycol 6956®, and Peppermint oil.

Another embodiment comprises cyclosporin A, Trycol 6961®, and Peppermint oil.

Another embodiment comprises cyclosporin A, Trycol 6964®, and Peppermint oil.

Another embodiment comprises cyclosporin A, Trycol 6969®, and Peppermint oil.

Another embodiment comprises cyclosporin A, Trycol 6974®, and Peppermint oil.

Another embodiment comprises cyclosporin A, Witbreak DRB-127®, and Peppermint oil.

Another embodiment comprises cyclosporin A, Witbreak DRB-127®, and Peppermint oil.

Another embodiment comprises cyclosporin A, Witconol NP Series®, and Peppermint oil.

Another embodiment comprises cyclosporin A, an Ethoxylated Aryl Phenol, and Peppermint oil.

Another embodiment comprises cyclosporin A, Soprophor BSU®, and Peppermint oil.

Another embodiment comprises cyclosporin A, Soprophor CY/8®, and Peppermint oil.

Another embodiment comprises cyclosporin A, Soprophor S/25®, and Peppermint oil.

Another embodiment comprises cyclosporin A, Witconol NIO®, and Peppermint oil.

Another embodiment comprises cyclosporin A, Witconol NIW®, and Peppermint oil.

Another embodiment comprises cyclosporin A, Witconol S-100®, and Peppermint oil.

Another embodiment comprises cyclosporin A, an Ethoxylated Fatty Acid, and Peppermint oil.

Another embodiment comprises cyclosporin A, Aldo PGHMS®, and Peppermint oil.

Another embodiment comprises cyclosporin A, Alkamuls TO-15/HR®, and Peppermint oil.

Another embodiment comprises cyclosporin A, Armotan AL-69-66®, and Peppermint oil.

Another embodiment comprises cyclosporin A, Cerasynt 840®, and Peppermint oil.

Another embodiment comprises cyclosporin A, Cerasynt 945®, and Peppermint oil.

Another embodiment comprises cyclosporin A, Crystal Inhibitor No. 5®, and Peppermint oil.

Another embodiment comprises cyclosporin A, DeThox Acid L-9®, and Peppermint oil.

Another embodiment comprises cyclosporin A, DeThox Acid S-8®, and Peppermint oil.

Another embodiment comprises cyclosporin A, Ethofat 242/25®, and Peppermint oil.

Another embodiment comprises cyclosporin A, Hydropalat 65®, and Peppermint oil.

Another embodiment comprises cyclosporin A, Lipo EGMS®, and Peppermint oil.

Another embodiment comprises cyclosporin A, Lipopeg 2 DL®, and Peppermint oil.

Another embodiment comprises cyclosporin A, Lipopeg 4 DL®, and Peppermint oil.

Another embodiment comprises cyclosporin A, Lipopeg 4-L®, and Peppermint oil.

Another embodiment comprises cyclosporin A, Lipopeg 39-S®, and Peppermint oil.

Another embodiment comprises cyclosporin A, Lipopeg 4-S®, and Peppermint oil.

Another embodiment comprises cyclosporin A, Lipopeg 10-S®, and Peppermint oil.

Another embodiment comprises cyclosporin A, Lipopeg 100-S®, and Peppermint oil.

Another embodiment comprises cyclosporin A, Lipopeg 6000 DS®, and Peppermint oil.

Another embodiment comprises cyclosporin A, Lumulse 40-L®, and Peppermint oil.

Another embodiment comprises cyclosporin A, Lumulse 40-S®, and Peppermint oil.

Another embodiment comprises cyclosporin A, Lumulse 42-L®, and Peppermint oil.

Another embodiment comprises cyclosporin A, Lumulse 42-S®, and Peppermint oil.

Another embodiment comprises cyclosporin A, Lumulse 100-S®, and Peppermint oil.

Another embodiment comprises cyclosporin A, Lumulse 602-S®, and Peppermint oil.

Another embodiment comprises cyclosporin A, Magrabar PGE-20-0®, and Peppermint oil.

Another embodiment comprises cyclosporin A, Magrabar PGE-20L®, and Peppermint oil.

Another embodiment comprises cyclosporin A, Magrabar PGE-20T®, and Peppermint oil.

Another embodiment comprises cyclosporin A, Magrabar PGE-22-0®, and Peppermint oil.

Another embodiment comprises cyclosporin A, Magrabar PGE-22L®, and Peppermint oil.

Another embodiment comprises cyclosporin A, Magrabar PGE-22T®, and Peppermint oil.

Another embodiment comprises cyclosporin A, Magrabar PGE-40-0®, and Peppermint oil.

Another embodiment comprises cyclosporin A, Magrabar PGE-40L®, and Peppermint oil.

Another embodiment comprises cyclosporin A, Magrabar PGE-40T®, and Peppermint oil.

Another embodiment comprises cyclosporin A, Magrabar PGE-42-0®, and Peppermint oil.

Another embodiment comprises cyclosporin A, Magrabar PGE-42L®, and Peppermint oil.

Another embodiment comprises cyclosporin A, Magrabar PGE-42T®, and Peppermint oil.

Another embodiment comprises cyclosporin A, Magrabar PGE-60-0®, and Peppermint oil.

Another embodiment comprises cyclosporin A, Magrabar PGE-60L®, and Peppermint oil.

Another embodiment comprises cyclosporin A, Magrabar PGE-60T®, and Peppermint oil.

Another embodiment comprises cyclosporin A, Magrabar PGE-62-0®, and Peppermint oil.

Another embodiment comprises cyclosporin A, Magrabar PGE-62L®, and Peppermint oil.

Another embodiment comprises cyclosporin A, Magrabar PGE-62T®, and Peppermint oil.

Another embodiment comprises cyclosporin A, Mapeg S-40K®, and Peppermint oil.

Another embodiment comprises cyclosporin A, Marlowet OTS®, and Peppermint oil.

Another embodiment comprises cyclosporin A, Naturechem PGR®, and Peppermint oil.

Another embodiment comprises cyclosporin A, PG No. 4®, and Peppermint oil.

Another embodiment comprises cyclosporin A, Renex 20®, and Peppermint oil.

Another embodiment comprises cyclosporin A, Ritox 52®, and Peppermint oil.

Another embodiment comprises cyclosporin A, Ritox 53®, and Peppermint oil.

Another embodiment comprises cyclosporin A, Ritox 59®, and Peppermint oil.

Another embodiment comprises cyclosporin A, Surfax 8916/A®, and Peppermint oil.

Another embodiment comprises cyclosporin A, Tego Acid S 40 P®, and Peppermint oil.

Another embodiment comprises cyclosporin A, Tego Acid S 100 P®, and Peppermint oil.

Another embodiment comprises cyclosporin A, Tween 20®, and Peppermint oil.

Another embodiment comprises cyclosporin A, Volpo 131®, and Peppermint oil.

Another embodiment comprises cyclosporin A, an Ethoxylated Fatty Ester or Oil, and Peppermint oil.

Another embodiment comprises cyclosporin A, Acconon 6-C10®, and Peppermint oil.

Another embodiment comprises cyclosporin A, Acconon CC-6®, and Peppermint oil.

Another embodiment comprises cyclosporin A, Acconon CO-7®, and Peppermint oil.

Another embodiment comprises cyclosporin A, Aldosperse 40/60 FG®, and Peppermint oil.

Another embodiment comprises cyclosporin A, Aldosperse ML-23®, and Peppermint oil.

Another embodiment comprises cyclosporin A, Aldosperse MS-20 FG®, and Peppermint oil.

Another embodiment comprises cyclosporin A, Alkamuls EL-620®, and Peppermint oil.

Another embodiment comprises cyclosporin A, Alkamuls EL-719®, and Peppermint oil.

Another embodiment comprises cyclosporin A, Alkamuls EL-985®, and Peppermint oil.

Another embodiment comprises cyclosporin A, Arlatone G®, and Peppermint oil.

Another embodiment comprises cyclosporin A, Arlatone T®, and Peppermint oil.

Another embodiment comprises cyclosporin A, Atlas G-1045A®, and Peppermint oil.

Another embodiment comprises cyclosporin A, Atlas G-1086®, and Peppermint oil.

Another embodiment comprises cyclosporin A, Atlas G-1087®, and Peppermint oil.

Another embodiment comprises cyclosporin A, Atlas G-1089®, and Peppermint oil.

Another embodiment comprises cyclosporin A, Atlas G-1096®, and Peppermint oil.

Another embodiment comprises cyclosporin A, Atlas G-1292®, and Peppermint oil.

Another embodiment comprises cyclosporin A, Atlas G-1293®, and Peppermint oil.

Another embodiment comprises cyclosporin A, Atlas G-1300®, and Peppermint oil.

Another embodiment comprises cyclosporin A, Atlas G-7076®, and Peppermint oil.

Another embodiment comprises cyclosporin A, Capmul EMG®, and Peppermint oil.

Another embodiment comprises cyclosporin A, Chemonic CO-40®, and Peppermint oil.

Another embodiment comprises cyclosporin A, Chemonic LI-3®, and Peppermint oil.

Another embodiment comprises cyclosporin A, Chemonic LI-7®, and Peppermint oil.

Another embodiment comprises cyclosporin A, Cirrasol GM®, and Peppermint oil.

Another embodiment comprises cyclosporin A, Cremophor CO 40®, and Peppermint oil.

Another embodiment comprises cyclosporin A, Cremophor CO 410®, and Peppermint oil.

Another embodiment comprises cyclosporin A, Cremophor EL®, and Peppermint oil.

Another embodiment comprises cyclosporin A, Cremophor GC7®, and Peppermint oil.

Another embodiment comprises cyclosporin A, Cremophor RH-40®, and Peppermint oil.

Another embodiment comprises cyclosporin A, Crovol A-40®, and Peppermint oil.

Another embodiment comprises cyclosporin A, Crovol A-70®, and Peppermint oil.

Another embodiment comprises cyclosporin A, Crovol M-70®, and Peppermint oil.

Another embodiment comprises cyclosporin A, Crovol PK-70®, and Peppermint oil.

Another embodiment comprises cyclosporin A, Cutina E-24®, and Peppermint oil.

Another embodiment comprises cyclosporin A, Dacospin 12-R®, and Peppermint oil.

Another embodiment comprises cyclosporin A, Dehymuls HRE-7®, and Peppermint oil.

Another embodiment comprises cyclosporin A, DeSonic 30C®, and Peppermint oil.

Another embodiment comprises cyclosporin A, DeSonic 36C®, and Peppermint oil.

Another embodiment comprises cyclosporin A, DeSonic 40C®, and Peppermint oil.

Another embodiment comprises cyclosporin A, Durfax 60®, and Peppermint oil.

Another embodiment comprises cyclosporin A, Durfax 65®, and Peppermint oil.

Another embodiment comprises cyclosporin A, Durfax 80®, and Peppermint oil.

Another embodiment comprises cyclosporin A, Durfax EOM®, and Peppermint oil.

Another embodiment comprises cyclosporin A, Eccoterge NF-2®, and Peppermint oil.

Another embodiment comprises cyclosporin A, Emulpon CO-360®, and Peppermint oil.

Another embodiment comprises cyclosporin A, Emulpon CO-550®, and Peppermint oil.

Another embodiment comprises cyclosporin A, Emulsogen EL®, and Peppermint oil.

Another embodiment comprises cyclosporin A, Emulsogen HCO 040®, and Peppermint oil.

Another embodiment comprises cyclosporin A, Emulsogen HCO 060®, and Peppermint oil.

Another embodiment comprises cyclosporin A, Emulsynt 1055®, and Peppermint oil.

Another embodiment comprises cyclosporin A, Ethox 3095®, and Peppermint oil.

Another embodiment comprises cyclosporin A, Eumulgin RO-40®, and Peppermint oil.

Another embodiment comprises cyclosporin A, Genapol G-260®, and Peppermint oil.

Another embodiment comprises cyclosporin A, Glycosperse L-20®, and Peppermint oil.

Another embodiment comprises cyclosporin A, Glycosperse O-5®, and Peppermint oil.

Another embodiment comprises cyclosporin A, Glycosperse O-20®, and Peppermint oil.

Another embodiment comprises cyclosporin A, Glycosperse O-20 FG®, and Peppermint oil.

Another embodiment comprises cyclosporin A, Glycosperse S-20®, and Peppermint oil.

Another embodiment comprises cyclosporin A, Glycosperse S-20 FG®, and Peppermint oil.

Another embodiment comprises cyclosporin A, Glycosperse TS-20®, and Peppermint oil.

Another embodiment comprises cyclosporin A, Glycosperse TS-20 FG®, and Peppermint oil.

Another embodiment comprises cyclosporin A, Hetan SL®, and Peppermint oil.

Another embodiment comprises cyclosporin A, Hetan So®, and Peppermint oil.

Another embodiment comprises cyclosporin A, Hetan SS®, and Peppermint oil.

Another embodiment comprises cyclosporin A, Hetoxide C-2®, and Peppermint oil.

Another embodiment comprises cyclosporin A, Hetoxide C-9®, and Peppermint oil.

Another embodiment comprises cyclosporin A, Hetoxide C-15®, and Peppermint oil.

Another embodiment comprises cyclosporin A, Hetoxide C-25®, and Peppermint oil.

Another embodiment comprises cyclosporin A, Hetoxide C-40®, and Peppermint oil.

Another embodiment comprises cyclosporin A, Hetoxide C-200®, and Peppermint oil.

Another embodiment comprises cyclosporin A, Hetoxide C-200-50%®, and Peppermint oil.

Another embodiment comprises cyclosporin A, Hetoxide GC-30®, and Peppermint oil.

Another embodiment comprises cyclosporin A, Hetoxide HC-60®, and Peppermint oil.

Another embodiment comprises cyclosporin A, Ice No. 2®, and Peppermint oil.

Another embodiment comprises cyclosporin A, Incrocas 30/40®, and Peppermint oil.

Another embodiment comprises cyclosporin A, Lexol EC®, and Peppermint oil.

Another embodiment comprises cyclosporin A, Lexol EO®, and Peppermint oil.

Another embodiment comprises cyclosporin A, Lipocol HCO-40®, and Peppermint oil.

Another embodiment comprises cyclosporin A, Lipocol HCO-60®, and Peppermint oil.

Another embodiment comprises cyclosporin A, Lipocol O-3 Special®, and Peppermint oil.

Another embodiment comprises cyclosporin A, Lipopeg 2-L®, and Peppermint oil.

Another embodiment comprises cyclosporin A, Lipopeg 4-DO®, and Peppermint oil.

Another embodiment comprises cyclosporin A, Lipopeg 4-DS®, and Peppermint oil.

Another embodiment comprises cyclosporin A, Lipovol GTB®, and Peppermint oil.

Another embodiment comprises cyclosporin A, Lonzest SML-20®, and Peppermint oil.

Another embodiment comprises cyclosporin A, Lonzest SMO-20®, and Peppermint oil.

Another embodiment comprises cyclosporin A, Lonzest SMS-20®, and Peppermint oil.

Another embodiment comprises cyclosporin A, Lonzest STO-20®, and Peppermint oil.

Another embodiment comprises cyclosporin A, Lonzest STS-20®, and Peppermint oil.

Another embodiment comprises cyclosporin A, Lumulse GR-40®, and Peppermint oil.

Another embodiment comprises cyclosporin A, Lumulse GRH-40®, and Peppermint oil.

Another embodiment comprises cyclosporin A, Lumulse POE (7) GML®, and Peppermint oil.

Another embodiment comprises cyclosporin A, Lumulse POE (12) Glyc®, and Peppermint oil.

Another embodiment comprises cyclosporin A, Lumulse POE (40) MS KP®, and Peppermint oil.

Another embodiment comprises cyclosporin A, Marlowet 4750®, and Peppermint oil.

Another embodiment comprises cyclosporin A, Marlowet LVS®, and Peppermint oil.

Another embodiment comprises cyclosporin A, Marlowet R 11®, and Peppermint oil.

Another embodiment comprises cyclosporin A, Marlowet R 40®, and Peppermint oil.

Another embodiment comprises cyclosporin A, Mazol 80 MGK®, and Peppermint oil.

Another embodiment comprises cyclosporin A, Nonionic Emulsifier T-9®, and Peppermint oil.

Another embodiment comprises cyclosporin A, Oronal LCG®, and Peppermint oil.

Another embodiment comprises cyclosporin A, Polyderm PPI-CO-200®, and Peppermint oil.

Another embodiment comprises cyclosporin A, Polyderm PPI-CO-40®, and Peppermint oil.

Another embodiment comprises cyclosporin A, Rewoderm LI 520-70®, and Peppermint oil.

Another embodiment comprises cyclosporin A, Ritapeg 150 DS®, and Peppermint oil.

Another embodiment comprises cyclosporin A, Softigen 767®, and Peppermint oil.

Another embodiment comprises cyclosporin A, Surfactol 318®, and Peppermint oil.

Another embodiment comprises cyclosporin A, Surfactol 365®, and Peppermint oil.

Another embodiment comprises cyclosporin A, Syn Lube 107®, and Peppermint oil.

Another embodiment comprises cyclosporin A, Syn Lube 728®, and Peppermint oil.

Another embodiment comprises cyclosporin A, Syn Lube 1632H®, and Peppermint oil.

Another embodiment comprises cyclosporin A, Syn Lube 6277-A®, and Peppermint oil.

Another embodiment comprises cyclosporin A, T-Det C-20®, and Peppermint oil.

Another embodiment comprises cyclosporin A, T-Det C-40®, and Peppermint oil.

Another embodiment comprises cyclosporin A, Tally 100 Plus®, and Peppermint oil.

Another embodiment comprises cyclosporin A, Uniperol EL®, and Peppermint oil.

Another embodiment comprises cyclosporin A, a Fatty Ester, and Peppermint oil.

Another embodiment comprises cyclosporin A, Actralube-Syn 147®, and Peppermint oil.

Another embodiment comprises cyclosporin A, Atlas G-1556®, and Peppermint oil.

Another embodiment comprises cyclosporin A, Atlas G-1564®, and Peppermint oil.

Another embodiment comprises cyclosporin A, Atlasol Base Oil S®, and Peppermint oil.

Another embodiment comprises cyclosporin A, Base ML®, and Peppermint oil.

Another embodiment comprises cyclosporin A, Base MT®, and Peppermint oil.

Another embodiment comprises cyclosporin A, Cerasynt 303®, and Peppermint oil.

Another embodiment comprises cyclosporin A, Dermol 1012®, and Peppermint oil.

Another embodiment comprises cyclosporin A, Kemester 4000®, and Peppermint oil.

Another embodiment comprises cyclosporin A, Lactipol S®, and Peppermint oil.

Another embodiment comprises cyclosporin A, Magrabar PGO®, and Peppermint oil.

Another embodiment comprises cyclosporin A, Mayco Base BFO®, and Peppermint oil.

Another embodiment comprises cyclosporin A, Methyl Linoleate®, and Peppermint oil.

Another embodiment comprises cyclosporin A, Pationic 122A®, and Peppermint oil.

Another embodiment comprises cyclosporin A, Pationic 138C®, and Peppermint oil.

Another embodiment comprises cyclosporin A, Pationic CSL®, and Peppermint oil.

Another embodiment comprises cyclosporin A, Pationic ISL®, and Peppermint oil.

Another embodiment comprises cyclosporin A, Pationic SBL®, and Peppermint oil.

Another embodiment comprises cyclosporin A, Pationic SSL®, and Peppermint oil.

Another embodiment comprises cyclosporin A, Ritasol®, and Peppermint oil.

Another embodiment comprises cyclosporin A, Tego Alkanol CS 20®, and Peppermint oil.

Another embodiment comprises cyclosporin A, Tego Alkanol L23 P®, and Peppermint oil.

Another embodiment comprises cyclosporin A, Tego Alkanol S2®, and Peppermint oil.

Another embodiment comprises cyclosporin A, Tego Alkanol S20 P®, and Peppermint oil.

Another embodiment comprises cyclosporin A, Triemulsifier 600 MS®, and Peppermint oil.

Another embodiment comprises cyclosporin A, a Glycerol Esters, and Peppermint oil.

Another embodiment comprises cyclosporin A, Agro #9 Wint SBO®, and Peppermint oil.

Another embodiment comprises cyclosporin A, Ahcovel Base 700®, and Peppermint oil.

Another embodiment comprises cyclosporin A, Aldo HMS FG®, and Peppermint oil.

Another embodiment comprises cyclosporin A, Aldo MLD®, and Peppermint oil.

Another embodiment comprises cyclosporin A, Aldo MLD FG®, and Peppermint oil.

Another embodiment comprises cyclosporin A, Aldo MO FG®, and Peppermint oil.

Another embodiment comprises cyclosporin A, Aldo MS®, and Peppermint oil.

Another embodiment comprises cyclosporin A, Aldo MS FG®, and Peppermint oil.

Another embodiment comprises cyclosporin A, Aldo MS LG FG®, and Peppermint oil.

Another embodiment comprises cyclosporin A, Aldo MSD®, and Peppermint oil.

Another embodiment comprises cyclosporin A, Aldo MSD FG®, and Peppermint oil.

Another embodiment comprises cyclosporin A, Aldosperse O-20 FG®, and Peppermint oil.

Another embodiment comprises cyclosporin A, Aldosperse TS-20 FG®, and Peppermint oil.

Another embodiment comprises cyclosporin A, Aldosperse TS-40 FG®, and Peppermint oil.

Another embodiment comprises cyclosporin A, Arlacel 165®, and Peppermint oil.

Another embodiment comprises cyclosporin A, Arlacel 186®, and Peppermint oil.

Another embodiment comprises cyclosporin A, Capmul GMO®, and Peppermint oil.

Another embodiment comprises cyclosporin A, Capmul GMS®, and Peppermint oil.

Another embodiment comprises cyclosporin A, Caprol 3GO®, and Peppermint oil.

Another embodiment comprises cyclosporin A, Caprol 3GVS®, and Peppermint oil.

Another embodiment comprises cyclosporin A, Caprol 6G2S®, and Peppermint oil.

Another embodiment comprises cyclosporin A, Caprol 10G40®, and Peppermint oil.

Another embodiment comprises cyclosporin A, Caprol 10G100®, and Peppermint oil.

Another embodiment comprises cyclosporin A, Caprol ET®, and Peppermint oil.

Another embodiment comprises cyclosporin A, Caprol PGE860®, and Peppermint oil.

Another embodiment comprises cyclosporin A, Cerasynt 945®, and Peppermint oil.

Another embodiment comprises cyclosporin A, Cerasynt GMS®, and Peppermint oil.

Another embodiment comprises cyclosporin A, Cerasynt Q®, and Peppermint oil.

Another embodiment comprises cyclosporin A, Cerasynt SD®, and Peppermint oil.

Another embodiment comprises cyclosporin A, Cerasynt WM®, and Peppermint oil.

Another embodiment comprises cyclosporin A, Chemsperse 14®, and Peppermint oil.

Another embodiment comprises cyclosporin A, Cremophor GO-32®, and Peppermint oil.

Another embodiment comprises cyclosporin A, Cremophor GS11®, and Peppermint oil.

Another embodiment comprises cyclosporin A, Cremophor GS-32®, and Peppermint oil.

Another embodiment comprises cyclosporin A, Cutina KD-16®, and Peppermint oil.

Another embodiment comprises cyclosporin A, Dehymuls PGPH®, and Peppermint oil.

Another embodiment comprises cyclosporin A, Dermol DGDIS®, and Peppermint oil.

Another embodiment comprises cyclosporin A, Dermol DGMIS®, and Peppermint oil.

Another embodiment comprises cyclosporin A, Dermol G-76®, and Peppermint oil.

Another embodiment comprises cyclosporin A, Dermol G-7DI®, and Peppermint oil.

Another embodiment comprises cyclosporin A, Dermol NGDI®, and Peppermint oil.

Another embodiment comprises cyclosporin A, Dermolan GLH®, and Peppermint oil.

Another embodiment comprises cyclosporin A, Drewmulse GMO®, and Peppermint oil.

Another embodiment comprises cyclosporin A, Drewpol 3-5-M®, and Peppermint oil.

Another embodiment comprises cyclosporin A, Durlac 100 W®, and Peppermint oil.

Another embodiment comprises cyclosporin A, Dur-Lo®, and Peppermint oil.

Another embodiment comprises cyclosporin A, Dynasan 118®, and Peppermint oil.

Another embodiment comprises cyclosporin A, EC-25®, and Peppermint oil.

Another embodiment comprises cyclosporin A, EM 40®, and Peppermint oil.

Another embodiment comprises cyclosporin A, Emerest 2400®, and Peppermint oil.

Another embodiment comprises cyclosporin A, Emerest 2452®, and Peppermint oil.

Another embodiment comprises cyclosporin A, Empilan G-26®, and Peppermint oil.

Another embodiment comprises cyclosporin A, Genapol TSM®, and Peppermint oil.

Another embodiment comprises cyclosporin A, Hostacerin DGI®, and Peppermint oil.

Another embodiment comprises cyclosporin A, Hostacerin DGL®, and Peppermint oil.

Another embodiment comprises cyclosporin A, Hostacerin DGMS®, and Peppermint oil.

Another embodiment comprises cyclosporin A, Hostacerin DGSB®, and Peppermint oil.

Another embodiment comprises cyclosporin A, Ice No. 2®, and Peppermint oil.

Another embodiment comprises cyclosporin A, Imwitor 742®, and Peppermint oil.

Another embodiment comprises cyclosporin A, Imwitor 780 K®, and Peppermint oil.

Another embodiment comprises cyclosporin A, Imwitor 960 Flakes®, and Peppermint oil.

Another embodiment comprises cyclosporin A, Isolan GI 34®, and Peppermint oil.

Another embodiment comprises cyclosporin A, Isolan GO 33®, and Peppermint oil.

Another embodiment comprises cyclosporin A, Kemester 1000®, and Peppermint oil.

Another embodiment comprises cyclosporin A, Kemester 2000®, and Peppermint oil.

Another embodiment comprises cyclosporin A, Kemester 2000®, and Peppermint oil.

Another embodiment comprises cyclosporin A, Kemester 6000SE®, and Peppermint oil.

Another embodiment comprises cyclosporin A, Lamecreme DGE 18®, and Peppermint oil.

Another embodiment comprises cyclosporin A, Lexemul 515®, and Peppermint oil.

Another embodiment comprises cyclosporin A, Lexemul 561®, and Peppermint oil.

Another embodiment comprises cyclosporin A, Lexemul AR®, and Peppermint oil.

Another embodiment comprises cyclosporin A, Lexemul AS®, and Peppermint oil.

Another embodiment comprises cyclosporin A, Lexemul GDL®, and Peppermint oil.

Another embodiment comprises cyclosporin A, Lexemul T®, and Peppermint oil.

Another embodiment comprises cyclosporin A, Lipomulse 165®, and Peppermint oil.

Another embodiment comprises cyclosporin A, Lumulse GML K®, and Peppermint oil.

Another embodiment comprises cyclosporin A, Lumulse GMO K®, and Peppermint oil.

Another embodiment comprises cyclosporin A, Lumulse GMR K®, and Peppermint oil.

Another embodiment comprises cyclosporin A, Lumulse GMT K®, and Peppermint oil.

Another embodiment comprises cyclosporin A, Magrabar GMC®, and Peppermint oil.

Another embodiment comprises cyclosporin A, Magrabar GMO-CK®, and Peppermint oil.

Another embodiment comprises cyclosporin A, Magrabar MDG-5050®, and Peppermint oil.

Another embodiment comprises cyclosporin A, Magrabar PGO-315®, and Peppermint oil.

Another embodiment comprises cyclosporin A, Magrabar PGO-1010®, and Peppermint oil.

Another embodiment comprises cyclosporin A, Mazol 300K®, and Peppermint oil.

Another embodiment comprises cyclosporin A, Mazol GMO-K®, and Peppermint oil.

Another embodiment comprises cyclosporin A, Mazol GMS-K®, and Peppermint oil.

Another embodiment comprises cyclosporin A, Mazol PGO31-K®, and Peppermint oil.

Another embodiment comprises cyclosporin A, Miglyol 812®, and Peppermint oil.

Another embodiment comprises cyclosporin A, Norfox 165C®, and Peppermint oil.

Another embodiment comprises cyclosporin A, Schercemol GMIS®, and Peppermint oil.

Another embodiment comprises cyclosporin A, Tegin®, and Peppermint oil.

Another embodiment comprises cyclosporin A, Tegin 4100 Pellets®, and Peppermint oil.

Another embodiment comprises cyclosporin A, Tegin M Pellets®, and Peppermint oil.

Another embodiment comprises cyclosporin A, Tegin OV®, and Peppermint oil.

Another embodiment comprises cyclosporin A, Teginacid H®, and Peppermint oil.

Another embodiment comprises cyclosporin A, Tego Cosmo P813®, and Peppermint oil.

Another embodiment comprises cyclosporin A, Wickenol 535®, and Peppermint oil.

Another embodiment comprises cyclosporin A, Witconbl 14®, and Peppermint oil.

Another embodiment comprises cyclosporin A, Witconol 14®, and Peppermint oil.

Another embodiment comprises cyclosporin A, Witconol 14F®, and Peppermint oil.

Another embodiment comprises cyclosporin A, Witconol 18L®, and Peppermint oil.

Another embodiment comprises cyclosporin A, Witconol GOT®, and Peppermint oil.

Another embodiment comprises cyclosporin A, Witconol MST®, and Peppermint oil.

Another embodiment comprises cyclosporin A, Witconol RHT®, and Peppermint oil.

Another embodiment comprises cyclosporin A, a Glycol Ester, and Peppermint oil.

Another embodiment comprises cyclosporin A, Alkamuls 600 DO®, and Peppermint oil.

Another embodiment comprises cyclosporin A, Alkamuls SEG®, and Peppermint oil.

Another embodiment comprises cyclosporin A, Atlas EM-2®, and Peppermint oil.

Another embodiment comprises cyclosporin A, Cerasynt IP®, and Peppermint oil.

Another embodiment comprises cyclosporin A, Cerasynt M®, and Peppermint oil.

Another embodiment comprises cyclosporin A, Cerasynt MN®, and Peppermint oil.

Another embodiment comprises cyclosporin A, Cerasynt PA®, and Peppermint oil.

Another embodiment comprises cyclosporin A, Chemsperse EGDS®, and Peppermint oil.

Another embodiment comprises cyclosporin A, Chemsperse EGMS®, and Peppermint oil.

Another embodiment comprises cyclosporin A, Colonial Monolaurin®, and Peppermint oil.

Another embodiment comprises cyclosporin A, DeMuls SGE-95®, and Peppermint oil.

Another embodiment comprises cyclosporin A, Eccoterge 200®, and Peppermint oil.

Another embodiment comprises cyclosporin A, Emerest 2380®, and Peppermint oil.

Another embodiment comprises cyclosporin A, Ethox 2610®, and Peppermint oil.

Another embodiment comprises cyclosporin A, Ethox DO-9®, and Peppermint oil.

Another embodiment comprises cyclosporin A, Ethox DO-14®, and Peppermint oil.

Another embodiment comprises cyclosporin A, Ethox SO-9®, and Peppermint oil.

Another embodiment comprises cyclosporin A, Fizul MD-318®, and Peppermint oil.

Another embodiment comprises cyclosporin A, Genapol EGDS-VHP®, and Peppermint oil.

Another embodiment comprises cyclosporin A, Genapol TS Powder®, and Peppermint oil.

Another embodiment comprises cyclosporin A, Hostacerin WO®, and Peppermint oil.

Another embodiment comprises cyclosporin A, Inversol 140®, and Peppermint oil.

Another embodiment comprises cyclosporin A, Kemester 104®, and Peppermint oil.

Another embodiment comprises cyclosporin A, Kemester 205®, and Peppermint oil.

Another embodiment comprises cyclosporin A, Kemester 226®, and Peppermint oil.

Another embodiment comprises cyclosporin A, Kemester 5221SE®, and Peppermint oil.

Another embodiment comprises cyclosporin A, Kemester EGDS®, and Peppermint oil.

Another embodiment comprises cyclosporin A, Lexemul EGDS®, and Peppermint oil.

Another embodiment comprises cyclosporin A, Lexemul EGMS®, and Peppermint oil.

Another embodiment comprises cyclosporin A, Lexemul P®, and Peppermint oil.

Another embodiment comprises cyclosporin A, Lipo DGLS®, and Peppermint oil.

Another embodiment comprises cyclosporin A, Lipo EGDS®, and Peppermint oil.

Another embodiment comprises cyclosporin A, Lipo PGMS®, and Peppermint oil.

Another embodiment comprises cyclosporin A, Liposorb S-4®, and Peppermint oil.

Another embodiment comprises cyclosporin A, Liposorb TO-20®, and Peppermint oil.

Another embodiment comprises cyclosporin A, Lumulse PGO®, and Peppermint oil.

Another embodiment comprises cyclosporin A, Mackester EGDS®, and Peppermint oil.

Another embodiment comprises cyclosporin A, Mackester EGMS®, and Peppermint oil.

Another embodiment comprises cyclosporin A, Mackester GSTP®, and Peppermint oil.

Another embodiment comprises cyclosporin A, Mackester Series®, and Peppermint oil.

Another embodiment comprises cyclosporin A, Magrabar PDG-50®, and Peppermint oil.

Another embodiment comprises cyclosporin A, Mapeg 6000 DS®, and Peppermint oil.

Another embodiment comprises cyclosporin A, Marlowet 4702®, and Peppermint oil.

Another embodiment comprises cyclosporin A, Monalube 305®, and Peppermint oil.

Another embodiment comprises cyclosporin A, Monalube 310®, and Peppermint oil.

Another embodiment comprises cyclosporin A, Monalube 315®, and Peppermint oil.

Another embodiment comprises cyclosporin A, Monalube 320®, and Peppermint oil.

Another embodiment comprises cyclosporin A, Monalube 325®, and Peppermint oil.

Another embodiment comprises cyclosporin A, Monalube 330®, and Peppermint oil.

Another embodiment comprises cyclosporin A, Naturechem PGHS®, and Peppermint oil.

Another embodiment comprises cyclosporin A, Polycastorol PLO-840®, and Peppermint oil.

Another embodiment comprises cyclosporin A, Polytex 10M®, and Peppermint oil.

Another embodiment comprises cyclosporin A, Ritasynt IP®, and Peppermint oil.

Another embodiment comprises cyclosporin A, Ross Chem PEG 600 DT®, and Peppermint oil;

Another embodiment comprises cyclosporin A, Schercemol PGMS®, and Peppermint oil.

Another embodiment comprises cyclosporin A, Sponto H-44C®, and Peppermint oil.

Another embodiment comprises cyclosporin A, Tegin G®, and Peppermint oil.

Another embodiment comprises cyclosporin A, Witbreak DGE-182®, and Peppermint oil.

Another embodiment comprises cyclosporin A, Witbreak DGE-182®, and Peppermint oil.

Another embodiment comprises cyclosporin A, Witbreak DRA-21®, and Peppermint oil.

Another embodiment comprises cyclosporin A, Witbreak DRA-21®, and Peppermint oil.

Another embodiment comprises cyclosporin A, Witbreak DRA-50®, and Peppermint oil.

Another embodiment comprises cyclosporin A, Witbreak DRA-50®, and Peppermint oil.

Another embodiment comprises cyclosporin A, Witconol F26-46®, and Peppermint oil.

Another embodiment comprises cyclosporin A, Witconol H-32®, and Peppermint oil.

Another embodiment comprises cyclosporin A, Witconol H-33®, and Peppermint oil.

Another embodiment comprises cyclosporin A, Witconol H-35A®, and Peppermint oil.

Another embodiment comprises cyclosporin A, Witconol RHP®, and Peppermint oil.

Another embodiment comprises cyclosporin A, a Lanolin-based Derivative, and Peppermint oil.

Another embodiment comprises cyclosporin A, Amerchol CAB®, and Peppermint oil.

Another embodiment comprises cyclosporin A, Amerchol L-101®, and Peppermint oil.

Another embodiment comprises cyclosporin A, Amerlate LFA-LO®, and Peppermint oil.

Another embodiment comprises cyclosporin A, Amerlate P®, and Peppermint oil.

Another embodiment comprises cyclosporin A, Barre Common Degras®, and Peppermint oil.

Another embodiment comprises cyclosporin A, Cholesterol NF®, and Peppermint oil.

Another embodiment comprises cyclosporin A, Crodalan AWS®, and Peppermint oil.

Another embodiment comprises cyclosporin A, Crodalan LA®, and Peppermint oil.

Another embodiment comprises cyclosporin A, Emery 1650®, and Peppermint oil.

Another embodiment comprises cyclosporin A, Emery 1650®, and Peppermint oil.

Another embodiment comprises cyclosporin A, Emery 1740®, and Peppermint oil.

Another embodiment comprises cyclosporin A, Emery 1740®, and Peppermint oil.

Another embodiment comprises cyclosporin A, Forlan 500®, and Peppermint oil.

Another embodiment comprises cyclosporin A, Forlan L®, and Peppermint oil.

Another embodiment comprises cyclosporin A, Laneto 50®, and Peppermint oil.

Another embodiment comprises cyclosporin A, Laneto 100®, and Peppermint oil.

Another embodiment comprises cyclosporin A, Laneto AWS®, and Peppermint oil.

Another embodiment comprises cyclosporin A, Lanfrax 1776®, and Peppermint oil.

Another embodiment comprises cyclosporin A, Lanfrax 1776®, and Peppermint oil.

Another embodiment comprises cyclosporin A, Lanogel 21®, and Peppermint oil.

Another embodiment comprises cyclosporin A, Lipolan®, and Peppermint oil.

Another embodiment comprises cyclosporin A, Lipolan 31®, and Peppermint oil.

Another embodiment comprises cyclosporin A, OHlan®, and Peppermint oil.

Another embodiment comprises cyclosporin A, Polychol 5®, and Peppermint oil.

Another embodiment comprises cyclosporin A, Polychol 15®, and Peppermint oil.

Another embodiment comprises cyclosporin A, Ritacetyl®, and Peppermint oil.

Another embodiment comprises cyclosporin A, Ritachol®, and Peppermint oil.

Another embodiment comprises cyclosporin A, Ritahydrox®, and Peppermint oil.

Another embodiment comprises cyclosporin A, Ritalafa®, and Peppermint oil.

Another embodiment comprises cyclosporin A, Ritalan®, and Peppermint oil.

Another embodiment comprises cyclosporin A, Ritalan AWS®, and Peppermint oil.

Another embodiment comprises cyclosporin A, Ritalan C®, and Peppermint oil.

Another embodiment comprises cyclosporin A, Ritawax®, and Peppermint oil.

Another embodiment comprises cyclosporin A, Ritawax AEO®, and Peppermint oil.

Another embodiment comprises cyclosporin A, Ritawax ALA®, and Peppermint oil.

Another embodiment comprises cyclosporin A, Solan/Solan 50/Super Solan®, and Peppermint oil.

Another embodiment comprises cyclosporin A, Super Hartolan/Hartolan®, and Peppermint oil.

Another embodiment comprises cyclosporin A, Supersat AWS-4®, and Peppermint oil.

Another embodiment comprises cyclosporin A, Supersat AWS-24®, and Peppermint oil.

Another embodiment comprises cyclosporin A, a Lecithin or Lecithin Derivative, and Peppermint oil.

Another embodiment comprises cyclosporin A, Alcolec®, and Peppermint oil.

Another embodiment comprises cyclosporin A, Lecithin®, and Peppermint oil.

Another embodiment comprises cyclosporin A, Lexin K®, and Peppermint oil.

Another embodiment comprises cyclosporin A, Natipide®, and Peppermint oil.

Another embodiment comprises cyclosporin A, a Lignin or Lignin Derivative, and Peppermint oil.

Another embodiment comprises cyclosporin A, Diwatex XP 9®, and Peppermint oil.

Another embodiment comprises cyclosporin A, Dynasperse LCD®, and Peppermint oil.

Another embodiment comprises cyclosporin A, Indulin SAL®, and Peppermint oil.

Another embodiment comprises cyclosporin A, Indulin W-1®,and Peppermint oil.

Another embodiment comprises cyclosporin A, Indulin W-5®, and Peppermint oil.

Another embodiment comprises cyclosporin A, Lignosol FTA®, and Peppermint oil.

Another embodiment comprises cyclosporin A, Lignosol SFX-65®, and Peppermint oil.

Another embodiment comprises cyclosporin A, Marasperse 52 CP®, and Peppermint oil.

Another embodiment comprises cyclosporin A, Marasperse AG®, and Peppermint oil.

Another embodiment comprises cyclosporin A, Marasperse CBOS-4®, and Peppermint oil.

Another embodiment comprises cyclosporin A, Ufoxane 2®, and Peppermint oil.

Another embodiment comprises cyclosporin A, a Methyl Ester, and Peppermint oil.

Another embodiment comprises cyclosporin A, E.B. Cleaner AK®, and Peppermint oil.

Another embodiment comprises cyclosporin A, Oleocal ME-70®, and Peppermint oil.

Another embodiment comprises cyclosporin A, Trade Name Company®, and Peppermint oil.

Another embodiment comprises cyclosporin A, Methyl Esters (cont'd)®, and Peppermint oil.

Another embodiment comprises cyclosporin A, Oleocal ME-92®, and Peppermint oil.

Another embodiment comprises cyclosporin A, Oleocal ME-112®, and Peppermint oil.

Another embodiment comprises cyclosporin A, Oleocal ME-130®, and Peppermint oil.

Another embodiment comprises cyclosporin A, a Monoglyceride or a Derivative thereof, and Peppermint oil.

Another embodiment comprises cyclosporin A, Dynacet 211®, and Peppermint oil.

Another embodiment comprises cyclosporin A, Hetsorb S-20®, and Peppermint oil.

Another embodiment comprises cyclosporin A, Imwitor 191®, and Peppermint oil.

Another embodiment comprises cyclosporin A, Imwitor 370®, and Peppermint oil.

Another embodiment comprises cyclosporin A, Imwitor 375®, and Peppermint oil.

Another embodiment comprises cyclosporin A, Imwitor 900®, and Peppermint oil.

Another embodiment comprises cyclosporin A, Imwitor 945®, and Peppermint oil.

Another embodiment comprises cyclosporin A, Imwitor 2020®, and Peppermint oil.

Another embodiment comprises cyclosporin A, Kemester 5500®, and Peppermint oil.

Another embodiment comprises cyclosporin A, Kemester 6000®, and Peppermint oil.

Another embodiment comprises cyclosporin A, Magrabar GMC®, and Peppermint oil.

Another embodiment comprises cyclosporin A, Magrabar GMO-CK®, and Peppermint oil.

Another embodiment comprises cyclosporin A, Magrabar GPC-10®, and Peppermint oil.

Another embodiment comprises cyclosporin A, Magrabar MDG-5050®, and Peppermint oil.

Another embodiment comprises cyclosporin A, Monalube 335®, and Peppermint oil.

Another embodiment comprises cyclosporin A, Monoglycerides Glyceryl Monestearate Archer Daniels Midland Company®, and Peppermint oil.

Another embodiment comprises cyclosporin A, Rita GMS®, and Peppermint oil.

Another embodiment comprises cyclosporin A, Ritamulse SCG®, and Peppermint oil.

Another embodiment comprises cyclosporin A, Softigen 701®, and Peppermint oil.

Another embodiment comprises cyclosporin A, Tally 100 Plus®, and Peppermint oil.

Another embodiment comprises cyclosporin A, a Polyethylene Glycol, and Peppermint oil.

Another embodiment comprises cyclosporin A, Emulgade PL 68/50®, and Peppermint oil.

Another embodiment comprises cyclosporin A, Lumulse PEG®, and Peppermint oil.

Another embodiment comprises cyclosporin A, Rhodasurf PEG-400®, and Peppermint oil.

Another embodiment comprises cyclosporin A, Rhodasurf PEG-600®, and Peppermint oil.

Another embodiment comprises cyclosporin A, Witconol PEG-400®, and Peppermint oil.

Another embodiment comprises cyclosporin A, a Polymeric Surfactant, and Peppermint oil.

Another embodiment comprises cyclosporin A, Acritamer PNC-EG®, and Peppermint oil.

Another embodiment comprises cyclosporin A, Ag-Rho DEP-775®, and Peppermint oil.

Another embodiment comprises cyclosporin A, APG 325N Glycoside®, and Peppermint oil.

Another embodiment comprises cyclosporin A, Aristoflex AVC®, and Peppermint oil.

Another embodiment comprises cyclosporin A, Aristoflex HMB®, and Peppermint oil.

Another embodiment comprises cyclosporin A, Burco NPS-225®, and Peppermint oil.

Another embodiment comprises cyclosporin A, Burco NPS-816®, and Peppermint oil.

Another embodiment comprises cyclosporin A, Chemccinate 5603®, and Peppermint oil.

Another embodiment comprises cyclosporin A, Cosmedia Guar C-261N®, and Peppermint oil.

Another embodiment comprises cyclosporin A, Gantrez S-95®, and Peppermint oil.

Another embodiment comprises cyclosporin A, Glucopon 220 UP®, and Peppermint oil.

Another embodiment comprises cyclosporin A, Glucopon 225 DK®, and Peppermint oil.

Another embodiment comprises cyclosporin A, Glucopon 425 N®, and Peppermint oil.

Another embodiment comprises cyclosporin A, Glucopon 600 UP®, and Peppermint oil.

Another embodiment comprises cyclosporin A, Glucopon 625 UP®, and Peppermint oil.

Another embodiment comprises cyclosporin A, Pemulen 1621®, and Peppermint oil.

Another embodiment comprises cyclosporin A, Pemulen 1622®, and Peppermint oil.

Another embodiment comprises cyclosporin A, Pemulen TR-1®, and Peppermint oil.

Another embodiment comprises cyclosporin A, Pemulen TR-2®, and Peppermint oil.

Another embodiment comprises cyclosporin A, Plantacare 818®, and Peppermint oil.

Another embodiment comprises cyclosporin A, Plantapon LGC Sorb®, and Peppermint oil.

Another embodiment comprises cyclosporin A, Plantaren 1200N®, and Peppermint oil.

Another embodiment comprises cyclosporin A, Plantaren 2000N®, and Peppermint oil.

Another embodiment comprises cyclosporin A, Viscolam AT 64®, and Peppermint oil.

Another embodiment comprises cyclosporin A, Viscolam AT 64P®, and Peppermint oil.

Another embodiment comprises cyclosporin A, Viscolam AT 100®, and Peppermint oil.

Another embodiment comprises cyclosporin A, Viscolam MAC 7®, and Peppermint oil.

Another embodiment comprises cyclosporin A, Viscolam SMC 20®, and Peppermint oil.

Another embodiment comprises cyclosporin A, Witbreak RTC-323®, and Peppermint oil.

Another embodiment comprises cyclosporin A, WSI 3700®, and Peppermint oil.

Another embodiment comprises cyclosporin A, a Propoxylated or Ethoxylated Fatty Acid, Alcohol, or Alkyl Phenol, and Peppermint oil.

Another embodiment comprises cyclosporin A, Antarox AA-60®, and Peppermint oil.

Another embodiment comprises cyclosporin A, Antarox LF-224®, and Peppermint oil.

Another embodiment comprises cyclosporin A, Burcomul DFE-45®, and Peppermint oil.

Another embodiment comprises cyclosporin A, Burcoterge LFE-1000®, and Peppermint oil.

Another embodiment comprises cyclosporin A, Chemal LF-25B®, and Peppermint oil.

Another embodiment comprises cyclosporin A, Chemal LF-40B®, and Peppermint oil.

Another embodiment comprises cyclosporin A, Dehypon LS-36®, and Peppermint oil.

Another embodiment comprises cyclosporin A, Dehypon LS-36®, and Peppermint oil.

Another embodiment comprises cyclosporin A, Dehypon LS-54®, and Peppermint oil.

Another embodiment comprises cyclosporin A, Dehypon LS-54®, and Peppermint oil.

Another embodiment comprises cyclosporin A, Delonic 100 VLF®, and Peppermint oil.

Another embodiment comprises cyclosporin A, Delonic LF-60 MOD®, and Peppermint oil.

Another embodiment comprises cyclosporin A, Empiderm B®, and Peppermint oil.

Another embodiment comprises cyclosporin A, Ethylan 1206®, and Peppermint oil.

Another embodiment comprises cyclosporin A, Ethylan NS-500K®, and Peppermint oil.

Another embodiment comprises cyclosporin A, Ethylan NS-500LQ®, and Peppermint oil.

Another embodiment comprises cyclosporin A, Genapol 1392®, and Peppermint oil.

Another embodiment comprises cyclosporin A, Genapol 2317®, and Peppermint oil.

Another embodiment comprises cyclosporin A, Genapol 26EP710®, and Peppermint oil.

Another embodiment comprises cyclosporin A, Genapol EP 1022®, and Peppermint oil.

Another embodiment comprises cyclosporin A, Genapol EP 1024®, and Peppermint oil.

Another embodiment comprises cyclosporin A, Genapol EP 6068®, and Peppermint oil.

Another embodiment comprises cyclosporin A, Genapol NP915®, and Peppermint oil.

Another embodiment comprises cyclosporin A, Kieralon MFB®, and Peppermint oil.

Another embodiment comprises cyclosporin A, Lumisolve CSA-80 V®, and Peppermint oil.

Another embodiment comprises cyclosporin A, Marlowet 5001®, and Peppermint oil.

Another embodiment comprises cyclosporin A, Marlox FK 64®, and Peppermint oil.

Another embodiment comprises cyclosporin A, Marlox MO 124®, and Peppermint oil.

Another embodiment comprises cyclosporin A, Marlox S 58®, and Peppermint oil.

Another embodiment comprises cyclosporin A, Nonatell 1003®, and Peppermint oil.

Another embodiment comprises cyclosporin A, Nonatell 1038®, and Peppermint oil.

Another embodiment comprises cyclosporin A, Nonatell 1052®, and Peppermint oil.

Another embodiment comprises cyclosporin A, Nonatell 1061®, and Peppermint oil.

Another embodiment comprises cyclosporin A, Nonatell 1075®, and Peppermint oil.

Another embodiment comprises cyclosporin A, Nonatell 1088®, and Peppermint oil.

Another embodiment comprises cyclosporin A, Nonatell 1123®, and Peppermint oil.

Another embodiment comprises cyclosporin A, Nonatell 1153®, and Peppermint oil.

Another embodiment comprises cyclosporin A, Nonatell 1161®, and Peppermint oil.

Another embodiment comprises cyclosporin A, Nonatell 1172®, and Peppermint oil.

Another embodiment comprises cyclosporin A, Nonatell 1181®, and Peppermint oil.

Another embodiment comprises cyclosporin A, Norfox 36®, and Peppermint oil.

Another embodiment comprises cyclosporin A, Procetyl AWS®, and Peppermint oil.

Another embodiment comprises cyclosporin A, Sandoxylate SX 412®, and Peppermint oil.

Another embodiment comprises cyclosporin A, Sandoxylate SX 418®, and Peppermint oil.

Another embodiment comprises cyclosporin A, Surfonic JL-80X®, and Peppermint oil.

Another embodiment comprises cyclosporin A, Surfonic JL-80X-B1®, and Peppermint oil.

Another embodiment comprises cyclosporin A, Surfonic L4-29X®, and Peppermint oil.

Another embodiment comprises cyclosporin A, Surfonic LF®, and Peppermint oil.

Another embodiment comprises cyclosporin A, T-Det A826®, and Peppermint oil.

Another embodiment comprises cyclosporin A, T-Det LF-416®, and Peppermint oil.

Another embodiment comprises cyclosporin A, Tergitol Min-Foam 1X®, and Peppermint oil.

Another embodiment comprises cyclosporin A, Tergitol Min-Foam 2X®, and Peppermint oil.

Another embodiment comprises cyclosporin A, Triton CF-21®, and Peppermint oil.

Another embodiment comprises cyclosporin A, Triton CF-76®, and Peppermint oil.

Another embodiment comprises cyclosporin A, Triton XL-80N®, and Peppermint oil.

Another embodiment comprises cyclosporin A, Witconol NS-98®, and Peppermint oil.

Another embodiment comprises cyclosporin A, Witconol NS-108LQ®, and Peppermint oil.

Another embodiment comprises cyclosporin A, Witconol NS-145®, and Peppermint oil.

Another embodiment comprises cyclosporin A, Witconol NS-179®, and Peppermint oil.

Another embodiment comprises cyclosporin A, a Protein-based Surfactant, and Peppermint oil.

Another embodiment comprises cyclosporin A, AminoFoam W®, and Peppermint oil.

Another embodiment comprises cyclosporin A, Amiter LGOD-2®, and Peppermint oil.

Another embodiment comprises cyclosporin A, Amiter LGS-2®, and Peppermint oil.

Another embodiment comprises cyclosporin A, Amiter LGS-5®, and Peppermint oil.

Another embodiment comprises cyclosporin A, Lamepon S®, and Peppermint oil.

Another embodiment comprises cyclosporin A, Lamepon S®, and Peppermint oil.

Another embodiment comprises cyclosporin A, Maypon 4C®, and Peppermint oil.

Another embodiment comprises cyclosporin A, May-Tein C®, and Peppermint oil.

Another embodiment comprises cyclosporin A, May-Tein CT®, and Peppermint oil.

Another embodiment comprises cyclosporin A, May-Tein KTS®, and Peppermint oil.

Another embodiment comprises cyclosporin A, May-Tein SY®, and Peppermint oil.

Another embodiment comprises cyclosporin A, Plantapon S®, and Peppermint oil.

Another embodiment comprises cyclosporin A, Proteol APL®, and Peppermint oil.

Another embodiment comprises cyclosporin A, Proteol OAT®, and Peppermint oil.

Another embodiment comprises cyclosporin A, Pyroter CPI-40®, and Peppermint oil.

Another embodiment comprises cyclosporin A, Pyroter GPI-25®, and Peppermint oil.

Another embodiment comprises cyclosporin A, Supro-Tein S®, and Peppermint oil.

Another embodiment comprises cyclosporin A, Supro-Tein V®, and Peppermint oil.

Another embodiment comprises cyclosporin A,® and Peppermint oil.

Another embodiment comprises cyclosporin A, a Sarcosine Derivative®, and Peppermint oil.

Another embodiment comprises cyclosporin A, Crodasinic LS-30®, and Peppermint oil.

Another embodiment comprises cyclosporin A, Vanseal CS®, and Peppermint oil.

Another embodiment comprises cyclosporin A, Vanseal LS®, and Peppermint oil.

Another embodiment comprises cyclosporin A, Vanseal MS®, and Peppermint oil.

Another embodiment comprises cyclosporin A, Vanseal NACS-30®, and Peppermint oil.

Another embodiment comprises cyclosporin A, Vanseal NALS-95®, and Peppermint oil.

Another embodiment comprises cyclosporin A, Vanseal OS®, and Peppermint oil.

Another embodiment comprises cyclosporin A,® and Peppermint oil.

Another embodiment comprises cyclosporin A, a Silicone-based Surfactant®, and Peppermint oil.

Another embodiment comprises cyclosporin A, Abil-B-9950®, and Peppermint oil.

Another embodiment comprises cyclosporin A, Abil Care 85®, and Peppermint oil.

Another embodiment comprises cyclosporin A, Abil EM 90®, and Peppermint oil.

Another embodiment comprises cyclosporin A, Abil EM 97®, and Peppermint oil.

Another embodiment comprises cyclosporin A, Abil WE-09®, and Peppermint oil.

Another embodiment comprises cyclosporin A, Dow Corning 1248 Fluid®, and Peppermint oil.

Another embodiment comprises cyclosporin A, Dow Corning 3225C®, and Peppermint oil.

Another embodiment comprises cyclosporin A, Dow Corning 5200®, and Peppermint oil.

Another embodiment comprises cyclosporin A, Dow Corning Q4-3667®, and Peppermint oil.

Another embodiment comprises cyclosporin A, Monasil PCA®, and Peppermint oil.

Another embodiment comprises cyclosporin A, Monasil PDM®, and Peppermint oil.

Another embodiment comprises cyclosporin A, Monasil PLN®, and Peppermint oil.

Another embodiment comprises cyclosporin A, Polyderm PPI-SI-WS®, and Peppermint oil.

Another embodiment comprises cyclosporin A, Troysol 380W®, and Peppermint oil.

Another embodiment comprises cyclosporin A, Troysol S366®, and Peppermint oil.

Another embodiment comprises cyclosporin A, a Sorbitan Derivative, and Peppermint oil.

Another embodiment comprises cyclosporin A, Alkamuls SML®, and Peppermint oil.

Another embodiment comprises cyclosporin A, Alkamuls SMO®, and Peppermint oil.

Another embodiment comprises cyclosporin A, Alkamuls STO®, and Peppermint oil.

Another embodiment comprises cyclosporin A, Arlacel 20®, and Peppermint oil.

Another embodiment comprises cyclosporin A, Arlacel 40®, and Peppermint oil.

Another embodiment comprises cyclosporin A, Arlacel 60®, and Peppermint oil.

Another embodiment comprises cyclosporin A, Arlacel 80®, and Peppermint oil.

Another embodiment comprises cyclosporin A, Arlacel C®, and Peppermint oil.

Another embodiment comprises cyclosporin A, Armul 21®, and Peppermint oil.

Another embodiment comprises cyclosporin A, Atlox 80®, and Peppermint oil.

Another embodiment comprises cyclosporin A, Atlox 847®, and Peppermint oil.

Another embodiment comprises cyclosporin A, Atlox 1045A®, and Peppermint oil.

Another embodiment comprises cyclosporin A, Canarcel 20®, and Peppermint oil.

Another embodiment comprises cyclosporin A, Canarcel 60®, and Peppermint oil.

Another embodiment comprises cyclosporin A, Canarcel 80®, and Peppermint oil.

Another embodiment comprises cyclosporin A, Canarcel TW 20®, and Peppermint oil.

Another embodiment comprises cyclosporin A, Canarcel TW 60®, and Peppermint oil.

Another embodiment comprises cyclosporin A, Canarcel TW 80®, and Peppermint oil.

Another embodiment comprises cyclosporin A, Coladet BSB-P®, and Peppermint oil.

Another embodiment comprises cyclosporin A, Customulse O-20®, and Peppermint oil.

Another embodiment comprises cyclosporin A, Dehymuls E®, and Peppermint oil.

Another embodiment comprises cyclosporin A, DeSotan SMO®, and Peppermint oil.

Another embodiment comprises cyclosporin A, DeSotan SMO-20®, and Peppermint oil.

Another embodiment comprises cyclosporin A, DeSotan SMT®, and Peppermint oil.

Another embodiment comprises cyclosporin A, DeSotan SMT-20®, and Peppermint oil.

Another embodiment comprises cyclosporin A, Durfax 60®, and Peppermint oil.

Another embodiment comprises cyclosporin A, Durfax 65®, and Peppermint oil.

Another embodiment comprises cyclosporin A, Durfax 80®, and Peppermint oil.

Another embodiment comprises cyclosporin A, Durtan 60®, and Peppermint oil.

Another embodiment comprises cyclosporin A, Durtan 65®, and Peppermint oil.

Another embodiment comprises cyclosporin A, Liposorb L®, and Peppermint oil.

Another embodiment comprises cyclosporin A, Liposorb L-10®, and Peppermint oil.

Another embodiment comprises cyclosporin A, Liposorb L-20®, and Peppermint oil.

Another embodiment comprises cyclosporin A, Liposorb O®, and Peppermint oil.

Another embodiment comprises cyclosporin A, Liposorb O-20®, and Peppermint oil.

Another embodiment comprises cyclosporin A, Liposorb P®, and Peppermint oil.

Another embodiment comprises cyclosporin A, Liposorb P-20®, and Peppermint oil.

Another embodiment comprises cyclosporin A, Liposorb S®, and Peppermint oil.

Another embodiment comprises cyclosporin A, Liposorb S-20®, and Peppermint oil.

Another embodiment comprises cyclosporin A, Liposorb SQO®, and Peppermint oil.

Another embodiment comprises cyclosporin A, Liposorb TO®, and Peppermint oil.

Another embodiment comprises cyclosporin A, Liposorb TS®, and Peppermint oil.

Another embodiment comprises cyclosporin A, Liposorb TS-20®, and Peppermint oil.

Another embodiment comprises cyclosporin A, Lumisorb PS®, and Peppermint oil.

Another embodiment comprises cyclosporin A, Lumisorb SMO (T)®, and Peppermint oil.

Another embodiment comprises cyclosporin A, Lumisorb SMS K®, and Peppermint oil.

Another embodiment comprises cyclosporin A, Lumisorb SSO®, and Peppermint oil.

Another embodiment comprises cyclosporin A, Lumisorb STS K®, and Peppermint oil.

Another embodiment comprises cyclosporin A, Lumisorb STT®, and Peppermint oil.

Another embodiment comprises cyclosporin A, Magrabar SMO®, and Peppermint oil.

Another embodiment comprises cyclosporin A, Magrabar SMO-VEG®, and Peppermint oil.

Another embodiment comprises cyclosporin A, Magrabar SMT®, and Peppermint oil.

Another embodiment comprises cyclosporin A, Magrabar STO®, and Peppermint oil.

Another embodiment comprises cyclosporin A, Miracare BC-27®, and Peppermint oil.

Another embodiment comprises cyclosporin A, Ritabate 20®, and Peppermint oil.

Another embodiment comprises cyclosporin A, Ritabate 40®, and Peppermint oil.

Another embodiment comprises cyclosporin A, Ritabate 60®, and Peppermint oil.

Another embodiment comprises cyclosporin A, Ritabate 80®, and Peppermint oil.

Another embodiment comprises cyclosporin A, T-Maz®, and Peppermint oil.

Another embodiment comprises cyclosporin A, Tego SML®, and Peppermint oil.

Another embodiment comprises cyclosporin A, Tego SML 20®, and Peppermint oil.

Another embodiment comprises cyclosporin A, Tego SMO 80 V®, and Peppermint oil.

Another embodiment comprises cyclosporin A, Tego SMO V®, and Peppermint oil.

Another embodiment comprises cyclosporin A, Tego SMS®, and Peppermint oil.

Another embodiment comprises cyclosporin A, Tego STO V®, and Peppermint oil.

Another embodiment comprises cyclosporin A, Tween 21®, and Peppermint oil.

Another embodiment comprises cyclosporin A, Tween 40®, and Peppermint oil.

Another embodiment comprises cyclosporin A, Tween 60®, and Peppermint oil.

Another embodiment comprises cyclosporin A, Tween 60 K®, and Peppermint oil.

Another embodiment comprises cyclosporin A, Tween 61®, and Peppermint oil.

Another embodiment comprises cyclosporin A, Tween 65®, and Peppermint oil.

Another embodiment comprises cyclosporin A, Tween 80®, and Peppermint oil.

Another embodiment comprises cyclosporin A, Tween 80 K®, and Peppermint oil.

Another embodiment comprises cyclosporin A, Tween 81®, and Peppermint oil.

Another embodiment comprises cyclosporin A, Tween 85®, and Peppermint oil.

Another embodiment comprises cyclosporin A, a Sucrose or Glucose Ester, or Derivative thereof, and Peppermint oil.

Another embodiment comprises cyclosporin A, DeSulf GOS-P-60WCG®, and Peppermint oil.

Another embodiment comprises cyclosporin A, Glucam E-20 Distearate®, and Peppermint oil.

Another embodiment comprises cyclosporin A, Glucamate DOE-120®, and Peppermint oil.

Another embodiment comprises cyclosporin A, Glucamate SSE-20®, and Peppermint oil.

Another embodiment comprises cyclosporin A, Glucate DO®, and Peppermint oil.

Another embodiment comprises cyclosporin A, Glucate SS®, and Peppermint oil.

Another embodiment comprises cyclosporin A, Glucopon 425 UP®, and Peppermint oil.

Another embodiment comprises cyclosporin A, Isolan IS®, and Peppermint oil.

Another embodiment comprises cyclosporin A, Mazon 40®, and Peppermint oil.

Another embodiment comprises cyclosporin A, Montanov 82®, and Peppermint oil.

Another embodiment comprises cyclosporin A, Montanov 202®, and Peppermint oil.

Another embodiment comprises cyclosporin A, Montanov S®, and Peppermint oil.

Another embodiment comprises cyclosporin A, Rheozan®, and Peppermint oil.

Another embodiment comprises cyclosporin A, Simulsol AS 48®, and Peppermint oil.

Another embodiment comprises cyclosporin A, Simulsol SL 4®, and Peppermint oil.

Another embodiment comprises cyclosporin A, Simulsol SL 10®, and Peppermint oil.

Another embodiment comprises cyclosporin A, Simulsol SL 11W®, and Peppermint oil.

Another embodiment comprises cyclosporin A, Simulsol SL 55®, and Peppermint oil.

Another embodiment comprises cyclosporin A, Suga Nate 100 and 160®, and Peppermint oil.

Another embodiment comprises cyclosporin A, Tego Care 450®, and Peppermint oil.

Another embodiment comprises cyclosporin A, Tego Care CG 90®, and Peppermint oil.

Another embodiment comprises cyclosporin A, Tego Care PS®, and Peppermint oil.

Another embodiment comprises cyclosporin A, Tegosoft PSE 141 G®, and Peppermint oil.

Another embodiment comprises cyclosporin A, Tegotens G 826®, and Peppermint oil.

Another embodiment comprises cyclosporin A, Triton BG-10 (70%)®, and Peppermint oil.

Another embodiment comprises cyclosporin A, Triton CG-110 (60%)®, and Peppermint oil.

Another embodiment comprises cyclosporin A, Wickenol 545®, and Peppermint oil.

Another embodiment comprises cyclosporin A, an alcohol, and Sunflower oil.

Another embodiment comprises cyclosporin A, Diglycerol®, and Sunflower oil.

Another embodiment comprises cyclosporin A, Hetoxide GT-80®, and Sunflower oil.

Another embodiment comprises cyclosporin A, Lexemul BEO®, and Sunflower oil.

Another embodiment comprises cyclosporin A, Polyglycerol-3®, and Sunflower oil.

Another embodiment comprises cyclosporin A, Redicote E Series®, and Sunflower oil.

Another embodiment comprises cyclosporin A, Simulsol OX 1005L®, and Sunflower oil.

Another embodiment comprises cyclosporin A, Stanfax 567®, and Sunflower oil.

Another embodiment comprises cyclosporin A, TA-1618®, and Sunflower oil.

Another embodiment comprises cyclosporin A, Witconol H-31A®, and Sunflower oil.

Another embodiment comprises cyclosporin A, an amine oxide, and Sunflower oil.

Another embodiment comprises cyclosporin A, AO-405®, and Sunflower oil.

Another embodiment comprises cyclosporin A, AO-455®, and Sunflower oil.

Another embodiment comprises cyclosporin A, AO 728 Special®, and Sunflower oil.

Another embodiment comprises cyclosporin A, Barlox 12®, and Sunflower oil.

Another embodiment comprises cyclosporin A, Barlox 14®, and Sunflower oil.

Another embodiment comprises cyclosporin A, Burcoxide Lo®, and Sunflower oil.

Another embodiment comprises cyclosporin A, Caloxamine LO®, and Sunflower oil.

Another embodiment comprises cyclosporin A, Chemoxide CAW®, and Sunflower oil.

Another embodiment comprises cyclosporin A, Chemoxide LM-30®, and Sunflower oil.

Another embodiment comprises cyclosporin A, Chemoxide LO®, and Sunflower oil.

Another embodiment comprises cyclosporin A, Chemoxide MO®, and Sunflower oil.

Another embodiment comprises cyclosporin A, Colalux CAO-35®, and Sunflower oil.

Another embodiment comprises cyclosporin A, Colalux LO®, and Sunflower oil.

Another embodiment comprises cyclosporin A, DeMox CAPO®, and Sunflower oil.

Another embodiment comprises cyclosporin A, DeMox CSG-30®, and Sunflower oil.

Another embodiment comprises cyclosporin A, DeMox LAO®, and Sunflower oil.

Another embodiment comprises cyclosporin A, Emcol L®, and Sunflower oil.

Another embodiment comprises cyclosporin A, Empigen OB®, and Sunflower oil.

Another embodiment comprises cyclosporin A, Empigen OS/A®, and Sunflower oil.

Another embodiment comprises cyclosporin A, Foamox CDO®, and Sunflower oil.

Another embodiment comprises cyclosporin A, Foamox DMM®, and Sunflower oil.

Another embodiment comprises cyclosporin A, Foamox DMS®, and Sunflower oil.

Another embodiment comprises cyclosporin A, Genaminox KC®, and Sunflower oil.

Another embodiment comprises cyclosporin A, Genaminox LA®, and Sunflower oil.

Another embodiment comprises cyclosporin A, Hartofoam SAO®, and Sunflower oil.

Another embodiment comprises cyclosporin A, Hartox DMCD®, and Sunflower oil.

Another embodiment comprises cyclosporin A, Lipowax DAT®, and Sunflower oil.

Another embodiment comprises cyclosporin A, Lipowax PB Pastilles®, and Sunflower oil.

Another embodiment comprises cyclosporin A, Mackamine C8®, and Sunflower oil.

Another embodiment comprises cyclosporin A, Mackamine C10®, and Sunflower oil.

Another embodiment comprises cyclosporin A, Mackamine C14®, and Sunflower oil.

Another embodiment comprises cyclosporin A, Mackamine CAO®, and Sunflower oil.

Another embodiment comprises cyclosporin A, Mackamine CO®, and Sunflower oil.

Another embodiment comprises cyclosporin A, Mackamine LO®, and Sunflower oil.

Another embodiment comprises cyclosporin A, Mackamine O2®, and Sunflower oil.

Another embodiment comprises cyclosporin A, Mackamine SAO®, and Sunflower oil.

Another embodiment comprises cyclosporin A, Mackamine SO®, and Sunflower oil.

Another embodiment comprises cyclosporin A, Mazox KCAO®, and Sunflower oil.

Another embodiment comprises cyclosporin A, Monalac MO®, and Sunflower oil.

Another embodiment comprises cyclosporin A, Norfox LDA®, and Sunflower oil.

Another embodiment comprises cyclosporin A, Rhodarox LO®, and Sunflower oil.

Another embodiment comprises cyclosporin A, Schercamox C-AA®, and Sunflower oil.

Another embodiment comprises cyclosporin A, Schercamox DMA®, and Sunflower oil.

Another embodiment comprises cyclosporin A, Schercamox DML®, and Sunflower oil.

Another embodiment comprises cyclosporin A, Schercamox DMM®, and Sunflower oil.

Another embodiment comprises cyclosporin A, Schercamox DMS®, and Sunflower oil.

Another embodiment comprises cyclosporin A, Tegotens DO®, and Sunflower oil.

Another embodiment comprises cyclosporin A, Tomah AO-14-2®, and Sunflower oil.

Another embodiment comprises cyclosporin A, Triaminox CDO®, and Sunflower oil.

Another embodiment comprises cyclosporin A, a block polymer, and Sunflower oil.

Another embodiment comprises cyclosporin A, AL 2070®, and Sunflower oil.

Another embodiment comprises cyclosporin A, Antarox 17-R-2®, and Sunflower oil.

Another embodiment comprises cyclosporin A, Antarox 25-R-2®, and Sunflower oil.

Another embodiment comprises cyclosporin A, Antarox 31-R-1®, and Sunflower oil.

Another embodiment comprises cyclosporin A, Antarox P-84®, and Sunflower oil.

Another embodiment comprises cyclosporin A, Antarox P-104/H®, and Sunflower oil.

Another embodiment comprises cyclosporin A, Arnox BP-Series®, and Sunflower oil.

Another embodiment comprises cyclosporin A, Chemonic 435®, and Sunflower oil.

Another embodiment comprises cyclosporin A, Chemonic D-25®, and Sunflower oil.

Another embodiment comprises cyclosporin A, Chemonic PL Series®, and Sunflower oil.

Another embodiment comprises cyclosporin A, Ethox L-121®, and Sunflower oil.

Another embodiment comprises cyclosporin A, Ethox L-122®, and Sunflower oil.

Another embodiment comprises cyclosporin A, Genapol PF-10®, and Sunflower oil.

Another embodiment comprises cyclosporin A, Genapol PF-20®, and Sunflower oil.

Another embodiment comprises cyclosporin A, Genapol PF-40A®, and Sunflower oil.

Another embodiment comprises cyclosporin A, Norfox 2-LF®, and Sunflower oil.

Another embodiment comprises cyclosporin A, Simulsol NW 342®, and Sunflower oil.

Another embodiment comprises cyclosporin A, T-Det BP-1®, and Sunflower oil.

Another embodiment comprises cyclosporin A, T-Det XD®, and Sunflower oil.

Another embodiment comprises cyclosporin A, T-Det XH®, and Sunflower oil.

Another embodiment comprises cyclosporin A, Triton CF-32®, and Sunflower oil.

Another embodiment comprises cyclosporin A, Witconol 171®, and Sunflower oil.

Another embodiment comprises cyclosporin A, Witconol 324®, and Sunflower oil.

Another embodiment comprises cyclosporin A, Witconol 324D®, and Sunflower oil.

Another embodiment comprises cyclosporin A, Witconol PD-2000®, and Sunflower oil.

Another embodiment comprises cyclosporin A, a Carboxylated Alcohol or Alkylphenol Ethoxylate, and Sunflower oil.

Another embodiment comprises cyclosporin A, Emcol CN-6®, and Sunflower oil.

Another embodiment comprises cyclosporin A, Ethcarb®, and Sunflower oil.

Another embodiment comprises cyclosporin A, Gemtex WNT-Conc®, and Sunflower oil.

Another embodiment comprises cyclosporin A, Incrodet TD7-C®, and Sunflower oil.

Another embodiment comprises cyclosporin A, Marlinat CM 105/80®, and Sunflower oil.

Another embodiment comprises cyclosporin A, Marlowet 1072®, and Sunflower oil.

Another embodiment comprises cyclosporin A, Marlowet 4530®, and Sunflower oil.

Another embodiment comprises cyclosporin A, Marlowet 4530 LF®, and Sunflower oil.

Another embodiment comprises cyclosporin A, Marlowet 4534®, and Sunflower oil.

Another embodiment comprises cyclosporin A, Marlowet 4538®, and Sunflower oil.

Another embodiment comprises cyclosporin A, Marlowet 4539®, and Sunflower oil.

Another embodiment comprises cyclosporin A, Marlowet 4539 LF®, and Sunflower oil.

Another embodiment comprises cyclosporin A, Marlowet 4541®, and Sunflower oil.

Another embodiment comprises cyclosporin A, Miranate LEC-80®, and Sunflower oil.

Another embodiment comprises cyclosporin A, Sandopan B®, and Sunflower oil.

Another embodiment comprises cyclosporin A, Sandopan B Modified®, and Sunflower oil.

Another embodiment comprises cyclosporin A, Sandopan LS-24 Gel®, and Sunflower oil.

Another embodiment comprises cyclosporin A, Surfine T-A®, and Sunflower oil.

Another embodiment comprises cyclosporin A, a Carboxylic Acid/Fatty Acid, and Sunflower oil.

Another embodiment comprises cyclosporin A, Colaterge RAM®, and Sunflower oil.

Another embodiment comprises cyclosporin A, Colatrope INC®, and Sunflower oil.

Another embodiment comprises cyclosporin A, Crodacid B®, and Sunflower oil.

Another embodiment comprises cyclosporin A, DeTrope CA-100®, and Sunflower oil.

Another embodiment comprises cyclosporin A, Latol MTO®, and Sunflower oil.

Another embodiment comprises cyclosporin A, Lumulse CC-33 K®, and Sunflower oil.

Another embodiment comprises cyclosporin A, Mulls 2218®, and Sunflower oil.

Another embodiment comprises cyclosporin A, OL-600®, and Sunflower oil.

Another embodiment comprises cyclosporin A, OL-800®, and Sunflower oil.

Another embodiment comprises cyclosporin A, R-910®, and Sunflower oil.

Another embodiment comprises cyclosporin A, S-210®, and Sunflower oil.

Another embodiment comprises cyclosporin A, Sandopan DTC Acid®, and Sunflower oil.

Another embodiment comprises cyclosporin A, Sandopan LS 24 N®, and Sunflower oil.

Another embodiment comprises cyclosporin A, Sandopan MA-18®, and Sunflower oil.

Another embodiment comprises cyclosporin A, an Ethoxylated Alcohol, and Sunflower oil.

Another embodiment comprises cyclosporin A, Adsee 799®, and Sunflower oil.

Another embodiment comprises cyclosporin A, Adsee 799®, and Sunflower oil.

Another embodiment comprises cyclosporin A, Alfonic 610-3.5®, and Sunflower oil.

Another embodiment comprises cyclosporin A, Alfonic 810-2®, and Sunflower oil.

Another embodiment comprises cyclosporin A, Alfonic 810-6®, and Sunflower oil.

Another embodiment comprises cyclosporin A, Alfonic 1012-3®, and Sunflower oil.

Another embodiment comprises cyclosporin A, Alfonic 1012-5®, and Sunflower oil.

Another embodiment comprises cyclosporin A, Alfonic 1216CO-1.5®, and Sunflower oil.

Another embodiment comprises cyclosporin A, Alfonic 1216CO-7®, and Sunflower oil.

Another embodiment comprises cyclosporin A, Alfonic 1412-3®, and Sunflower oil.

Another embodiment comprises cyclosporin A, Alfonic 1412-7®, and Sunflower oil.

Another embodiment comprises cyclosporin A, Arlasolve 200®, and Sunflower oil.

Another embodiment comprises cyclosporin A, Arlasolve 200 Liquid®, and Sunflower oil.

Another embodiment comprises cyclosporin A, Armix 180-C®, and Sunflower oil.

Another embodiment comprises cyclosporin A, Armix 183®, and Sunflower oil.

Another embodiment comprises cyclosporin A, Armul 2404®, and Sunflower oil.

Another embodiment comprises cyclosporin A, Armul 2404®, and Sunflower oil.

Another embodiment comprises cyclosporin A, Atlas EMJ-C®, and Sunflower oil.

Another embodiment comprises cyclosporin A, Atlas G-2109®, and Sunflower oil.

Another embodiment comprises cyclosporin A, Atlas G-3886®, and Sunflower oil.

Another embodiment comprises cyclosporin A, Atlas G-3890®, and Sunflower oil.

Another embodiment comprises cyclosporin A, Bio Soft E-200®, and Sunflower oil.

Another embodiment comprises cyclosporin A, Bio Soft E-300®, and Sunflower oil.

Another embodiment comprises cyclosporin A, Bio Soft E-400®, and Sunflower oil.

Another embodiment comprises cyclosporin A, Bio Soft EN 600®, and Sunflower oil.

Another embodiment comprises cyclosporin A, Bio Soft TD-400®, and Sunflower oil.

Another embodiment comprises cyclosporin A, Bio Soft TD-630®, and Sunflower oil.

Another embodiment comprises cyclosporin A, Brij 30®, and Sunflower oil.

Another embodiment comprises cyclosporin A, Brij 52®, and Sunflower oil.

Another embodiment comprises cyclosporin A, Brij 56®, and Sunflower oil.

Another embodiment comprises cyclosporin A, Brij 58®, and Sunflower oil.

Another embodiment comprises cyclosporin A, Brij 72®, and Sunflower oil.

Another embodiment comprises cyclosporin A, Brij 76®, and Sunflower oil.

Another embodiment comprises cyclosporin A, Brij 78®, and Sunflower oil.

Another embodiment comprises cyclosporin A, Brij 93®, and Sunflower oil.

Another embodiment comprises cyclosporin A, Brij 97®, and Sunflower oil.

Another embodiment comprises cyclosporin A, Brij 98®, and Sunflower oil.

Another embodiment comprises cyclosporin A, Brij 700®, and Sunflower oil.

Another embodiment comprises cyclosporin A, Brij 700 S®, and Sunflower oil.

Another embodiment comprises cyclosporin A, Brij 721®, and Sunflower oil.

Another embodiment comprises cyclosporin A, Brij 721 S®, and Sunflower oil.

Another embodiment comprises cyclosporin A, Burcoterge CDG®, and Sunflower oil.

Another embodiment comprises cyclosporin A, Canasol AT 600®, and Sunflower oil.

Another embodiment comprises cyclosporin A, Canasol AT 800®, and Sunflower oil.

Another embodiment comprises cyclosporin A, Canasol AT 1200®, and Sunflower oil.

Another embodiment comprises cyclosporin A, Canasol BJ 35®, and Sunflower oil.

Another embodiment comprises cyclosporin A, Canasol BJ 36®, and Sunflower oil.

Another embodiment comprises cyclosporin A, Canasol BJ 52®, and Sunflower oil.

Another embodiment comprises cyclosporin A, Canasol BJ 58®, and Sunflower oil.

Another embodiment comprises cyclosporin A, Canasol BJ 72®, and Sunflower oil.

Another embodiment comprises cyclosporin A, Canasol BJ 78®, and Sunflower oil.

Another embodiment comprises cyclosporin A, Canasol BJ 98®, and Sunflower oil.

Another embodiment comprises cyclosporin A, Canasol BJ 307®, and Sunflower oil.

Another embodiment comprises cyclosporin A, Cerfak 1400®, and Sunflower oil.

Another embodiment comprises cyclosporin A, Cetomacrogol 1000 BP®, and Sunflower oil.

Another embodiment comprises cyclosporin A, Chemonic C-2®, and Sunflower oil.

Another embodiment comprises cyclosporin A, Chemonic C-10®, and Sunflower oil.

Another embodiment comprises cyclosporin A, Chemonic C-20®, and Sunflower oil.

Another embodiment comprises cyclosporin A, Chemonic CT-12®, and Sunflower oil.

Another embodiment comprises cyclosporin A, Chemonic CT-20®, and Sunflower oil.

Another embodiment comprises cyclosporin A, Chemonic CT-30®, and Sunflower oil.

Another embodiment comprises cyclosporin A, Chemonic CT-55®, and Sunflower oil.

Another embodiment comprises cyclosporin A, Chemonic G-7®, and Sunflower oil.

Another embodiment comprises cyclosporin A, Chemonic G-26®, and Sunflower oil.

Another embodiment comprises cyclosporin A, Chemonic L-4®, and Sunflower oil.

Another embodiment comprises cyclosporin A, Chemonic L-7®, and Sunflower oil.

Another embodiment comprises cyclosporin A, Chemonic L-12®, and Sunflower oil.

Another embodiment comprises cyclosporin A, Chemonic L-23®, and Sunflower oil.

Another embodiment comprises cyclosporin A, Chemonic O-2®, and Sunflower oil.

Another embodiment comprises cyclosporin A, Chemonic O-5®, and Sunflower oil.

Another embodiment comprises cyclosporin A, Chemonic O-10®, and Sunflower oil.

Another embodiment comprises cyclosporin A, Chemonic O-20®, and Sunflower oil.

Another embodiment comprises cyclosporin A, Chemonic S-2®, and Sunflower oil.

Another embodiment comprises cyclosporin A, Chemonic S-10®, and Sunflower oil.

Another embodiment comprises cyclosporin A, Chemonic S-20®, and Sunflower oil.

Another embodiment comprises cyclosporin A, Colamulse FE®, and Sunflower oil.

Another embodiment comprises cyclosporin A, Cremophor A 20®, and Sunflower oil.

Another embodiment comprises cyclosporin A, Cremophor SA 2®, and Sunflower oil.

Another embodiment comprises cyclosporin A, Dehydol 100®, and Sunflower oil.

Another embodiment comprises cyclosporin A, Dehydol O-4®, and Sunflower oil.

Another embodiment comprises cyclosporin A, Delonic C-18®, and Sunflower oil.

Another embodiment comprises cyclosporin A, DeSonic 6T®, and Sunflower oil.

Another embodiment comprises cyclosporin A, DeSonic 9D®, and Sunflower oil.

Another embodiment comprises cyclosporin A, DeSonic 9T®, and Sunflower oil.

Another embodiment comprises cyclosporin A, DeSonic 12D®, and Sunflower oil.

Another embodiment comprises cyclosporin A, DeSonic 12T®, and Sunflower oil.

Another embodiment comprises cyclosporin A, DeSonic 15T®, and Sunflower oil.

Another embodiment comprises cyclosporin A, DeSonic TDA-9®, and Sunflower oil.

Another embodiment comprises cyclosporin A, DeThox GLG-7®, and Sunflower oil.

Another embodiment comprises cyclosporin A, DeThox GLG-26®, and Sunflower oil.

Another embodiment comprises cyclosporin A, DeThox LA-4®, and Sunflower oil.

Another embodiment comprises cyclosporin A, DeThox LA-23®, and Sunflower oil.

Another embodiment comprises cyclosporin A, DeThox SA-80®, and Sunflower oil.

Another embodiment comprises cyclosporin A, Disponil O5®, and Sunflower oil.

Another embodiment comprises cyclosporin A, Eccoterge EO-41B®, and Sunflower oil.

Another embodiment comprises cyclosporin A, Empilan KA2.5/90®, and Sunflower oil.

Another embodiment comprises cyclosporin A, Empilan KA5/90®, and Sunflower oil.

Another embodiment comprises cyclosporin A, Empilan KM-20®, and Sunflower oil.

Another embodiment comprises cyclosporin A, Empilan KM-50®, and Sunflower oil.

Another embodiment comprises cyclosporin A, Empilan L-23®, and Sunflower oil.

Another embodiment comprises cyclosporin A, Ethylan 25-3®, and Sunflower oil.

Another embodiment comprises cyclosporin A, Ethylan 1204®, and Sunflower oil.

Another embodiment comprises cyclosporin A, Ethylan DA-4®, and Sunflower oil.

Another embodiment comprises cyclosporin A, Ethylan LA-230®, and Sunflower oil.

Another embodiment comprises cyclosporin A, Ethylan SN®, and Sunflower oil.

Another embodiment comprises cyclosporin A, Ethylan TD-60®, and Sunflower oil.

Another embodiment comprises cyclosporin A, Ethylan TD-100®, and Sunflower oil.

Another embodiment comprises cyclosporin A, Ethylan TD-1407®, and Sunflower oil.

Another embodiment comprises cyclosporin A, Eumulgin B1®, and Sunflower oil.

Another embodiment comprises cyclosporin A, Eumulgin B2®, and Sunflower oil.

Another embodiment comprises cyclosporin A, Eumulgin B3®, and Sunflower oil.

Another embodiment comprises cyclosporin A, Eumulgin O-10®, and Sunflower oil.

Another embodiment comprises cyclosporin A, Flo Mo 80/20®, and Sunflower oil.

Another embodiment comprises cyclosporin A, Flo Mo Low Foam®, and Sunflower oil.

Another embodiment comprises cyclosporin A, Forlan C-24®, and Sunflower oil.

Another embodiment comprises cyclosporin A, Genapol 1454®, and Sunflower oil.

Another embodiment comprises cyclosporin A, Genapol BA-020®, and Sunflower oil.

Another embodiment comprises cyclosporin A, Genapol BA-040®, and Sunflower oil.

Another embodiment comprises cyclosporin A, Genapol C-100®, and Sunflower oil.

Another embodiment comprises cyclosporin A, Genapol DA 060®, and Sunflower oil.

Another embodiment comprises cyclosporin A, Genapol HS 020®, and Sunflower oil.

Another embodiment comprises cyclosporin A, Genapol HS 200®, and Sunflower oil.

Another embodiment comprises cyclosporin A, Genapol ID-040®, and Sunflower oil.

Another embodiment comprises cyclosporin A, Genapol ID-060®, and Sunflower oil.

Another embodiment comprises cyclosporin A, Genapol ID-090®, and Sunflower oil.

Another embodiment comprises cyclosporin A, Genapol LA 010®, and Sunflower oil.

Another embodiment comprises cyclosporin A, Genapol LA 020®, and Sunflower oil.

Another embodiment comprises cyclosporin A, Genapol LA 030®, and Sunflower oil.

Another embodiment comprises cyclosporin A, Genapol LA 040®, and Sunflower oil.

Another embodiment comprises cyclosporin A, Genapol LA 050®, and Sunflower oil.

Another embodiment comprises cyclosporin A, Genapol LA 060®, and Sunflower oil.

Another embodiment comprises cyclosporin A, Genapol LA 070®, and Sunflower oil.

Another embodiment comprises cyclosporin A, Genapol LA 070S®, and Sunflower oil.

Another embodiment comprises cyclosporin A, Genapol LA 230®, and Sunflower oil.

Another embodiment comprises cyclosporin A, Genapol O020®, and Sunflower oil.

Another embodiment comprises cyclosporin A, Genapol O050®, and Sunflower oil.

Another embodiment comprises cyclosporin A, Genapol O100®, and Sunflower oil.

Another embodiment comprises cyclosporin A, Genapol O200®, and Sunflower oil.

Another embodiment comprises cyclosporin A, Genapol SA 030®, and Sunflower oil.

Another embodiment comprises cyclosporin A, Genapol SA 120®, and Sunflower oil.

Another embodiment comprises cyclosporin A, Genapol T-020®, and Sunflower oil.

Another embodiment comprises cyclosporin A, Genapol UD-030®, and Sunflower oil.

Another embodiment comprises cyclosporin A, Genapol UD-050®, and Sunflower oil.

Another embodiment comprises cyclosporin A, Genapol UD-070®, and Sunflower oil.

Another embodiment comprises cyclosporin A, Genapol UD-079®, and Sunflower oil.

Another embodiment comprises cyclosporin A, Genapol UD-080®, and Sunflower oil.

Another embodiment comprises cyclosporin A, Genapol UD-110®, and Sunflower oil.

Another embodiment comprises cyclosporin A, Genapol X 030®, and Sunflower oil.

Another embodiment comprises cyclosporin A, Genapol X 050®, and Sunflower oil.

Another embodiment comprises cyclosporin A, Genapol X 060®, and Sunflower oil.

Another embodiment comprises cyclosporin A, Genapol X 070®, and Sunflower oil.

Another embodiment comprises cyclosporin A, Genapol X 080®, and Sunflower oil.

Another embodiment comprises cyclosporin A, Genapol X 100®, and Sunflower oil.

Another embodiment comprises cyclosporin A, Genapol X 159®, and Sunflower oil.

Another embodiment comprises cyclosporin A, Generol 122 E5®, and Sunflower oil.

Another embodiment comprises cyclosporin A, Generol 122 E25®, and Sunflower oil.

Another embodiment comprises cyclosporin A, Hostacerin T-3®, and Sunflower oil.

Another embodiment comprises cyclosporin A, Iconol LF 110®, and Sunflower oil.

Another embodiment comprises cyclosporin A, Incropol CS-20®, and Sunflower oil.

Another embodiment comprises cyclosporin A, Lexemul CS-20®, and Sunflower oil.

Another embodiment comprises cyclosporin A, Liponic EG-1®, and Sunflower oil.

Another embodiment comprises cyclosporin A, Lipowax D®, and Sunflower oil.

Another embodiment comprises cyclosporin A, Lipowax G®, and Sunflower oil.

Another embodiment comprises cyclosporin A, Lipowax NI®, and Sunflower oil.

Another embodiment comprises cyclosporin A, Lipowax P®, and Sunflower oil.

Another embodiment comprises cyclosporin A, Lipowax P-31®, and Sunflower oil.

Another embodiment comprises cyclosporin A, Lipowax PR®, and Sunflower oil.

Another embodiment comprises cyclosporin A, Lumulse CS-20®, and Sunflower oil.

Another embodiment comprises cyclosporin A, Macol CSA-20®, and Sunflower oil.

Another embodiment comprises cyclosporin A, Marlox B 24/50®, and Sunflower oil.

Another embodiment comprises cyclosporin A, Mazawet 77®, and Sunflower oil.

Another embodiment comprises cyclosporin A, Norfox 1713®, and Sunflower oil.

Another embodiment comprises cyclosporin A, Norfox 2579®, and Sunflower oil.

Another embodiment comprises cyclosporin A, Norfox Lo Foam®, and Sunflower oil.

Another embodiment comprises cyclosporin A, Promulgen D®, and Sunflower oil.

Another embodiment comprises cyclosporin A, Promulgen G®, and Sunflower oil.

Another embodiment comprises cyclosporin A, Renex 30®, and Sunflower oil.

Another embodiment comprises cyclosporin A, Renex 36®, and Sunflower oil.

Another embodiment comprises cyclosporin A, Rhodasurf A 24®, and Sunflower oil.

Another embodiment comprises cyclosporin A, Rhodasurf AAE-10®, and Sunflower oil.

Another embodiment comprises cyclosporin A, Rhodasurf BEH-25®, and Sunflower oil.

Another embodiment comprises cyclosporin A, Rhodasurf BEH-40®, and Sunflower oil.

Another embodiment comprises cyclosporin A, Rhodasurf DA 530®, and Sunflower oil.

Another embodiment comprises cyclosporin A, Rhodasurf DA 630®, and Sunflower oil.

Another embodiment comprises cyclosporin A, Rhodasurf DA 639®, and Sunflower oil.

Another embodiment comprises cyclosporin A, Rhodasurf LAN-23®, and Sunflower oil.

Another embodiment comprises cyclosporin A, Rhodasurf ON-870®, and Sunflower oil.

Another embodiment comprises cyclosporin A, Rhodasun ON-877®, and Sunflower oil.

Another embodiment comprises cyclosporin A, Rhodasurf TB-970 FLK®, and Sunflower oil.

Another embodiment comprises cyclosporin A, Ritacet-20®, and Sunflower oil.

Another embodiment comprises cyclosporin A, Ritachol 1000®, and Sunflower oil.

Another embodiment comprises cyclosporin A, Ritachol 2000®, and Sunflower oil.

Another embodiment comprises cyclosporin A, Ritachol 5000®, and Sunflower oil.

Another embodiment comprises cyclosporin A, Ritox 35®, and Sunflower oil.

Another embodiment comprises cyclosporin A, Surfonic DA-4®, and Sunflower oil.

Another embodiment comprises cyclosporin A, Surfonic DA-6®, and Sunflower oil.

Another embodiment comprises cyclosporin A, Surfonic L46-7®, and Sunflower oil.

Another embodiment comprises cyclosporin A, Surfonic POA®, and Sunflower oil.

Another embodiment comprises cyclosporin A, Synthrapol KB®, and Sunflower oil.

Another embodiment comprises cyclosporin A, Teginacid®, and Sunflower oil.

Another embodiment comprises cyclosporin A, Teginacid C®, and Sunflower oil.

Another embodiment comprises cyclosporin A, Tegotens EC 11®, and Sunflower oil.

Another embodiment comprises cyclosporin A, Tinegal NA®, and Sunflower oil.

Another embodiment comprises cyclosporin A, Tomadol 400®, and Sunflower oil.

Another embodiment comprises cyclosporin A, Tomadol 600®, and Sunflower oil.

Another embodiment comprises cyclosporin A, Tomadol 900®, and Sunflower oil.

Another embodiment comprises cyclosporin A, Uniperol O®, and Sunflower oil.

Another embodiment comprises cyclosporin A, Witconol SN Series®, and Sunflower oil.

Another embodiment comprises cyclosporin A, an Ethoxylated Alkylphenol, and Sunflower oil.

Another embodiment comprises cyclosporin A, Antarox LF-222®, and Sunflower oil.

Another embodiment comprises cyclosporin A, Atlox 775®, and Sunflower oil.

Another embodiment comprises cyclosporin A, Caloxylate N-9®, and Sunflower oil.

Another embodiment comprises cyclosporin A, Canasol NF-1000®, and Sunflower oil.

Another embodiment comprises cyclosporin A, Canasol NF-3000®, and Sunflower oil.

Another embodiment comprises cyclosporin A, Canasol NF-3070®, and Sunflower oil.

Another embodiment comprises cyclosporin A, Canasol OF 1670®, and Sunflower oil.

Another embodiment comprises cyclosporin A, Canasol OF 2570®, and Sunflower oil.

Another embodiment comprises cyclosporin A, Canasol OF 4070®, and Sunflower oil.

Another embodiment comprises cyclosporin A, Chemax DNP-8®, and Sunflower oil.

Another embodiment comprises cyclosporin A, Chemax DNP-18®, and Sunflower oil.

Another embodiment comprises cyclosporin A, Chemax DNP-150/50®, and Sunflower oil.

Another embodiment comprises cyclosporin A, DeSonic 1.5N®, and Sunflower oil.

Another embodiment comprises cyclosporin A, DeSonic 4N®, and Sunflower oil.

Another embodiment comprises cyclosporin A, DeSonic 5N®, and Sunflower oil.

Another embodiment comprises cyclosporin A, DeSonic 6D®, and Sunflower oil.

Another embodiment comprises cyclosporin A, DeSonic 6N®, and Sunflower oil.

Another embodiment comprises cyclosporin A, DeSonic 7N®, and Sunflower oil.

Another embodiment comprises cyclosporin A, DeSonic 9N®, and Sunflower oil.

Another embodiment comprises cyclosporin A, DeSonic 10D®, and Sunflower oil.

Another embodiment comprises cyclosporin A, DeSonic 11N®, and Sunflower oil.

Another embodiment comprises cyclosporin A, DeSonic 12N®, and Sunflower oil.

Another embodiment comprises cyclosporin A, DeSonic 15N®, and Sunflower oil.

Another embodiment comprises cyclosporin A, DeSonic 20N®, and Sunflower oil.

Another embodiment comprises cyclosporin A, Eccoscour RC®, and Sunflower oil.

Another embodiment comprises cyclosporin A, Eccoterge EO-100®, and Sunflower oil.

Another embodiment comprises cyclosporin A, Emulsifier 632/90%®, and Sunflower oil.

Another embodiment comprises cyclosporin A, Geronol AG-821®, and Sunflower oil.

Another embodiment comprises cyclosporin A, Gradonic N-95®, and Sunflower oil.

Another embodiment comprises cyclosporin A, Hetoxide NP-4®, and Sunflower oil.

Another embodiment comprises cyclosporin A, Hetoxide NP-30®, and Sunflower oil.

Another embodiment comprises cyclosporin A, Hostapal N-100®, and Sunflower oil.

Another embodiment comprises cyclosporin A, Hostapal N-110®, and Sunflower oil.

Another embodiment comprises cyclosporin A, Igepal CTA-639W®, and Sunflower oil.

Another embodiment comprises cyclosporin A, Igepal DAP-9®, and Sunflower oil.

Another embodiment comprises cyclosporin A, Igepal OD-410®, and Sunflower oil.

Another embodiment comprises cyclosporin A, Igepal SS-837®, and Sunflower oil.

Another embodiment comprises cyclosporin A, Lipocol NP-9 USP®, and Sunflower oil.

Another embodiment comprises cyclosporin A, Macol DNP-10®, and Sunflower oil.

Another embodiment comprises cyclosporin A, Marlophen NP 5®, and Sunflower oil.

Another embodiment comprises cyclosporin A, Marlophen P 1®, and Sunflower oil.

Another embodiment comprises cyclosporin A, Surfonic NB®, and Sunflower oil.

Another embodiment comprises cyclosporin A, Surfonic OPB-307®, and Sunflower oil.

Another embodiment comprises cyclosporin A, Surfonic OPB-407®, and Sunflower oil.

Another embodiment comprises cyclosporin A, Syn Fac 334®, and Sunflower oil.

Another embodiment comprises cyclosporin A, Syn Fac 8216®, and Sunflower oil.

Another embodiment comprises cyclosporin A, Triton N-57®, and Sunflower oil.

Another embodiment comprises cyclosporin A, Trycol 6956®, and Sunflower oil.

Another embodiment comprises cyclosporin A, Trycol 6961®, and Sunflower oil.

Another embodiment comprises cyclosporin A, Trycol 6964®, and Sunflower oil.

Another embodiment comprises cyclosporin A, Trycol 6969®, and Sunflower oil.

Another embodiment comprises cyclosporin A, Trycol 6974®, and Sunflower oil.

Another embodiment comprises cyclosporin A, Witbreak DRB-127®, and Sunflower oil.

Another embodiment comprises cyclosporin A, Witbreak DRB-127®, and Sunflower oil.

Another embodiment comprises cyclosporin A, Witconol NP Series®, and Sunflower oil.

Another embodiment comprises cyclosporin A, an Ethoxylated Aryl Phenol, and Sunflower oil.

Another embodiment comprises cyclosporin A, Soprophor BSU®, and Sunflower oil.

Another embodiment comprises cyclosporin A, Soprophor CY/8®, and Sunflower oil.

Another embodiment comprises cyclosporin A, Soprophor S/25®, and Sunflower oil.

Another embodiment comprises cyclosporin A, Witconol NIO®, and Sunflower oil.

Another embodiment comprises cyclosporin A, Witconol NIW®, and Sunflower oil.

Another embodiment comprises cyclosporin A, Witconol S-100®, and Sunflower oil.

Another embodiment comprises cyclosporin A, an Ethoxylated Fatty Acid, and Sunflower oil.

Another embodiment comprises cyclosporin A, Aldo PGHMS®, and Sunflower oil.

Another embodiment comprises cyclosporin A, Alkamuls TO-15/HR®, and Sunflower oil.

Another embodiment comprises cyclosporin A, Armotan AL-69-66®, and Sunflower oil.

Another embodiment comprises cyclosporin A, Cerasynt 840®, and Sunflower oil.

Another embodiment comprises cyclosporin A, Cerasynt 945®, and Sunflower oil.

Another embodiment comprises cyclosporin A, Crystal Inhibitor No. 5®, and Sunflower oil.

Another embodiment comprises cyclosporin A, DeThox Acid L-9®, and Sunflower oil.

Another embodiment comprises cyclosporin A, DeThox Acid S-8®, and Sunflower oil.

Another embodiment comprises cyclosporin A, Ethofat 242/25®, and Sunflower oil.

Another embodiment comprises cyclosporin A, Hydropalat 65®, and Sunflower oil.

Another embodiment comprises cyclosporin A, Lipo EGMS®, and Sunflower oil.

Another embodiment comprises cyclosporin A, Lipopeg 2 DL®, and Sunflower oil.

Another embodiment comprises cyclosporin A, Lipopeg 4 DL®, and Sunflower oil.

Another embodiment comprises cyclosporin A, Lipopeg 4-L®, and Sunflower oil.

Another embodiment comprises cyclosporin A, Lipopeg 39-S®, and Sunflower oil.

Another embodiment comprises cyclosporin A, Lipopeg 4-S®, and Sunflower oil.

Another embodiment comprises cyclosporin A, Lipopeg 10-S®, and Sunflower oil.

Another embodiment comprises cyclosporin A, Lipopeg 100-S®, and Sunflower oil.

Another embodiment comprises cyclosporin A, Lipopeg 6000 DS®, and Sunflower oil.

Another embodiment comprises cyclosporin A, Lumulse 40-L®, and Sunflower oil.

Another embodiment comprises cyclosporin A, Lumulse 40-S®, and Sunflower oil.

Another embodiment comprises cyclosporin A, Lumulse 42-L®, and Sunflower oil.

Another embodiment comprises cyclosporin A, Lumulse 42-S®, and Sunflower oil.

Another embodiment comprises cyclosporin A, Lumulse 100-S®, and Sunflower oil.

Another embodiment comprises cyclosporin A, Lumulse 602-S®, and Sunflower oil.

Another embodiment comprises cyclosporin A, Magrabar PGE-20-0®, and Sunflower oil.

Another embodiment comprises cyclosporin A, Magrabar PGE-20L®, and Sunflower oil.

Another embodiment comprises cyclosporin A, Magrabar PGE-20T®, and Sunflower oil.

Another embodiment comprises cyclosporin A, Magrabar PGE-22-0®, and Sunflower oil.

Another embodiment comprises cyclosporin A, Magrabar PGE-22L®, and Sunflower oil.

Another embodiment comprises cyclosporin A, Magrabar PGE-22T®, and Sunflower oil.

Another embodiment comprises cyclosporin A, Magrabar PGE-40-0®, and Sunflower oil.

Another embodiment comprises cyclosporin A, Magrabar PGE-40L®, and Sunflower oil.

Another embodiment comprises cyclosporin A, Magrabar PGE-40T®, and Sunflower oil.

Another embodiment comprises cyclosporin A, Magrabar PGE-42-0®, and Sunflower oil.

Another embodiment comprises cyclosporin A, Magrabar PGE-42L®, and Sunflower oil.

Another embodiment comprises cyclosporin A, Magrabar PGE-42T®, and Sunflower oil.

Another embodiment comprises cyclosporin A, Magrabar PGE-60-0®, and Sunflower oil.

Another embodiment comprises cyclosporin A, Magrabar PGE-60L®, and Sunflower oil.

Another embodiment comprises cyclosporin A, Magrabar PGE-60T®, and Sunflower oil.

Another embodiment comprises cyclosporin A, Magrabar PGE-62-0®, and Sunflower oil.

Another embodiment comprises cyclosporin A, Magrabar PGE-62L®, and Sunflower oil.

Another embodiment comprises cyclosporin A, Magrabar PGE-62T®, and Sunflower oil.

Another embodiment comprises cyclosporin A, Mapeg S-40K®, and Sunflower oil.

Another embodiment comprises cyclosporin A, Marlowet OTS®, and Sunflower oil.

Another embodiment comprises cyclosporin A, Naturechem PGR®, and Sunflower oil.

Another embodiment comprises cyclosporin A, PG No. 4®, and Sunflower oil.

Another embodiment comprises cyclosporin A, Renex 20®, and Sunflower oil.

Another embodiment comprises cyclosporin A, Ritox 52®, and Sunflower oil.

Another embodiment comprises cyclosporin A, Ritox 53®, and Sunflower oil.

Another embodiment comprises cyclosporin A, Ritox 59®, and Sunflower oil.

Another embodiment comprises cyclosporin A, Surfax 8916/A®, and Sunflower oil.

Another embodiment comprises cyclosporin A, Tego Acid S 40 P®, and Sunflower oil.

Another embodiment comprises cyclosporin A, Tego Acid S 100 P®, and Sunflower oil.

Another embodiment comprises cyclosporin A, Tween 20®, and Sunflower oil.

Another embodiment comprises cyclosporin A, Volpo 131®, and Sunflower oil.

Another embodiment comprises cyclosporin A, an Ethoxylated Fatty Ester or Oil, and Sunflower oil.

Another embodiment comprises cyclosporin A, Acconon 6-C10®, and Sunflower oil.

Another embodiment comprises cyclosporin A, Acconon CC-6®, and Sunflower oil.

Another embodiment comprises cyclosporin A, Acconon CO-7®, and Sunflower oil.

Another embodiment comprises cyclosporin A, Aldosperse 40/60 FG®, and Sunflower oil.

Another embodiment comprises cyclosporin A, Aldosperse ML-23®, and Sunflower oil.

Another embodiment comprises cyclosporin A, Aldosperse MS-20 FG®, and Sunflower oil.

Another embodiment comprises cyclosporin A, Alkamuls EL-620®, and Sunflower oil.

Another embodiment comprises cyclosporin A, Alkamuls EL-719®, and Sunflower oil.

Another embodiment comprises cyclosporin A, Alkamuls EL-985®, and Sunflower oil.

Another embodiment comprises cyclosporin A, Arlatone G®, and Sunflower oil.

Another embodiment comprises cyclosporin A, Arlatone T®, and Sunflower oil.

Another embodiment comprises cyclosporin A, Atlas G-1045A®, and Sunflower oil.

Another embodiment comprises cyclosporin A, Atlas G-1086®, and Sunflower oil.

Another embodiment comprises cyclosporin A, Atlas G-1087®, and Sunflower oil.

Another embodiment comprises cyclosporin A, Atlas G-1089®, and Sunflower oil.

Another embodiment comprises cyclosporin A, Atlas G-1096®, and Sunflower oil.

Another embodiment comprises cyclosporin A, Atlas G-1292®, and Sunflower oil.

Another embodiment comprises cyclosporin A, Atlas G-1293®, and Sunflower oil.

Another embodiment comprises cyclosporin A, Atlas G-1300®, and Sunflower oil.

Another embodiment comprises cyclosporin A, Atlas G-7076®, and Sunflower oil.

Another embodiment comprises cyclosporin A, Capmul EMG®, and Sunflower oil.

Another embodiment comprises cyclosporin A, Chemonic CO-40®, and Sunflower oil.

Another embodiment comprises cyclosporin A, Chemonic LI-3®, and Sunflower oil.

Another embodiment comprises cyclosporin A, Chemonic LI-7®, and Sunflower oil.

Another embodiment comprises cyclosporin A, Cirrasol GM®, and Sunflower oil.

Another embodiment comprises cyclosporin A, Cremophor CO 40®, and Sunflower oil.

Another embodiment comprises cyclosporin A, Cremophor CO 410®, and Sunflower oil.

Another embodiment comprises cyclosporin A, Cremophor EL®, and Sunflower oil.

Another embodiment comprises cyclosporin A, Cremophor GC7®, and Sunflower oil.

Another embodiment comprises cyclosporin A, Cremophor RH-40®, and Sunflower oil.

Another embodiment comprises cyclosporin A, Crovol A-40®, and Sunflower oil.

Another embodiment comprises cyclosporin A, Crovol A-70®, and Sunflower oil.

Another embodiment comprises cyclosporin A, Crovol M-70®, and Sunflower oil.

Another embodiment comprises cyclosporin A, Crovol PK-70®, and Sunflower oil.

Another embodiment comprises cyclosporin A, Cutina E-24®, and Sunflower oil.

Another embodiment comprises cyclosporin A, Dacospin 12-R®, and Sunflower oil.

Another embodiment comprises cyclosporin A, Dehymuls HRE-7®, and Sunflower oil.

Another embodiment comprises cyclosporin A, DeSonic 30C®, and Sunflower oil.

Another embodiment comprises cyclosporin A, DeSonic 36C®, and Sunflower oil.

Another embodiment comprises cyclosporin A, DeSonic 40C®, and Sunflower oil.

Another embodiment comprises cyclosporin A, Durfax 60®, and Sunflower oil.

Another embodiment comprises cyclosporin A, Durfax 65®, and Sunflower oil.

Another embodiment comprises cyclosporin A, Durfax 80®, and Sunflower oil.

Another embodiment comprises cyclosporin A, Durfax EOM®, and Sunflower oil.

Another embodiment comprises cyclosporin A, Eccoterge NF-2®, and Sunflower oil.

Another embodiment comprises cyclosporin A, Emulpon CO-360®, and Sunflower oil.

Another embodiment comprises cyclosporin A, Emulpon CO-550®, and Sunflower oil.

Another embodiment comprises cyclosporin A, Emulsogen EL®, and Sunflower oil.

Another embodiment comprises cyclosporin A, Emulsogen HCO 040®, and Sunflower oil.

Another embodiment comprises cyclosporin A, Emulsogen HCO 060®, and Sunflower oil.

Another embodiment comprises cyclosporin A, Emulsynt 1055®, and Sunflower oil.

Another embodiment comprises cyclosporin A, Ethox 3095®, and Sunflower oil.

Another embodiment comprises cyclosporin A, Eumulgin RO-40®, and Sunflower oil.

Another embodiment comprises cyclosporin A, Genapol G-260®, and Sunflower oil.

Another embodiment comprises cyclosporin A, Glycosperse L-20®, and Sunflower oil.

Another embodiment comprises cyclosporin A, Glycosperse O-5®, and Sunflower oil.

Another embodiment comprises cyclosporin A, Glycosperse O-20®, and Sunflower oil.

Another embodiment comprises cyclosporin A, Glycosperse O-20 FG®, and Sunflower oil.

Another embodiment comprises cyclosporin A, Glycosperse S-20®, and Sunflower oil.

Another embodiment comprises cyclosporin A, Glycosperse S-20 FG®, and Sunflower oil.

Another embodiment comprises cyclosporin A, Glycosperse TS-20®, and Sunflower oil.

Another embodiment comprises cyclosporin A, Glycosperse TS-20 FG®, and Sunflower oil.

Another embodiment comprises cyclosporin A, Hetan SL®, and Sunflower oil.

Another embodiment comprises cyclosporin A, Hetan SO®, and Sunflower oil.

Another embodiment comprises cyclosporin A, Hetan SS®, and Sunflower oil.

Another embodiment comprises cyclosporin A, Hetoxide C-2®, and Sunflower oil.

Another embodiment comprises cyclosporin A, Hetoxide C-9®, and Sunflower oil.

Another embodiment comprises cyclosporin A, Hetoxide C-15®, and Sunflower oil.

Another embodiment comprises cyclosporin A, Hetoxide C-25®, and Sunflower oil.

Another embodiment comprises cyclosporin A, Hetoxide C-40®, and Sunflower oil.

Another embodiment comprises cyclosporin A, Hetoxide C-200®, and Sunflower oil.

Another embodiment comprises cyclosporin A, Hetoxide C-200-50%®, and Sunflower oil.

Another embodiment comprises cyclosporin A, Hetoxide GC-30®, and Sunflower oil.

Another embodiment comprises cyclosporin A, Hetoxide HC-60®, and Sunflower oil.

Another embodiment comprises cyclosporin A, Ice No. 2®, and Sunflower oil.

Another embodiment comprises cyclosporin A, Incrocas 30/40®, and Sunflower oil.

Another embodiment comprises cyclosporin A, Lexol EC®, and Sunflower oil.

Another embodiment comprises cyclosporin A, Lexol EO®, and Sunflower oil.

Another embodiment comprises cyclosporin A, Lipocol HCO-40®, and Sunflower oil.

Another embodiment comprises cyclosporin A, Lipocol HCO-60®, and Sunflower oil.

Another embodiment comprises cyclosporin A, Lipocol O-3 Special®, and Sunflower oil.

Another embodiment comprises cyclosporin A, Lipopeg 2-L®, and Sunflower oil.

Another embodiment comprises cyclosporin A, Lipopeg 4-DO®, and Sunflower oil.

Another embodiment comprises cyclosporin A, Lipopeg 4-DS®, and Sunflower oil.

Another embodiment comprises cyclosporin A, Lipovol GTB®, and Sunflower oil.

Another embodiment comprises cyclosporin A, Lonzest SML-20®, and Sunflower oil.

Another embodiment comprises cyclosporin A, Lonzest SMO-20®, and Sunflower oil.

Another embodiment comprises cyclosporin A, Lonzest SMS-20®, and Sunflower oil.

Another embodiment comprises cyclosporin A, Lonzest STO-20®, and Sunflower oil.

Another embodiment comprises cyclosporin A, Lonzest STS-20®, and Sunflower oil.

Another embodiment comprises cyclosporin A, Lumulse GR-40®, and Sunflower oil.

Another embodiment comprises cyclosporin A, Lumulse GRH-40®, and Sunflower oil.

Another embodiment comprises cyclosporin A, Lumulse POE (7) GML®, and Sunflower oil.

Another embodiment comprises cyclosporin A, Lumulse POE (12) Glyc®, and Sunflower oil.

Another embodiment comprises cyclosporin A, Lumulse POE (40) MS KP®, and Sunflower oil.

Another embodiment comprises cyclosporin A, Marlowet 4750®, and Sunflower oil.

Another embodiment comprises cyclosporin A, Marlowet LVS®, and Sunflower oil.

Another embodiment comprises cyclosporin A, Marlowet R 11®, and Sunflower oil.

Another embodiment comprises cyclosporin A, Marlowet R 40®, and Sunflower oil.

Another embodiment comprises cyclosporin A, Mazol 80 MGK®, and Sunflower oil.

Another embodiment comprises cyclosporin A, Nonionic Emulsifier T-9®, and Sunflower oil.

Another embodiment comprises cyclosporin A, Oronal LCG®, and Sunflower oil.

Another embodiment comprises cyclosporin A, Polyderm PPI-CO-200®, and Sunflower oil.

Another embodiment comprises cyclosporin A, Polyderm PPI-CO-40®, and Sunflower oil.

Another embodiment comprises cyclosporin A, Rewoderm LI 520-70®, and Sunflower oil.

Another embodiment comprises cyclosporin A, Ritapeg 150 DS®, and Sunflower oil.

Another embodiment comprises cyclosporin A, Softigen 767®, and Sunflower oil.

Another embodiment comprises cyclosporin A, Surfactol 318®, and Sunflower oil.

Another embodiment comprises cyclosporin A, Surfactol 365®, and Sunflower oil.

Another embodiment comprises cyclosporin A, Syn Lube 107®, and Sunflower oil.

Another embodiment comprises cyclosporin A, Syn Lube 728®, and Sunflower oil.

Another embodiment comprises cyclosporin A, Syn Lube 1632H®, and Sunflower oil.

Another embodiment comprises cyclosporin A, Syn Lube 6277-A®, and Sunflower oil.

Another embodiment comprises cyclosporin A, T-Det C-20®, and Sunflower oil.

Another embodiment comprises cyclosporin A, T-Det C-40®, and Sunflower oil.

Another embodiment comprises cyclosporin A, Tally 100 Plus®, and Sunflower oil.

Another embodiment comprises cyclosporin A, Uniperol EL®, and Sunflower oil.

Another embodiment comprises cyclosporin A, a Fatty Ester, and Sunflower oil.

Another embodiment comprises cyclosporin A, Actralube-Syn 147®, and Sunflower oil.

Another embodiment comprises cyclosporin A, Atlas G-1556®, and Sunflower oil.

Another embodiment comprises cyclosporin A, Atlas G-1564®, and Sunflower oil.

Another embodiment comprises cyclosporin A, Atlasol Base Oil S®, and Sunflower oil.

Another embodiment comprises cyclosporin A, Base ML®, and Sunflower oil.

Another embodiment comprises cyclosporin A, Base MT®, and Sunflower oil.

Another embodiment comprises cyclosporin A, Cerasynt 303®, and Sunflower oil.

Another embodiment comprises cyclosporin A, Dermol 1012®, and Sunflower oil.

Another embodiment comprises cyclosporin A, Kemester 4000®, and Sunflower oil.

Another embodiment comprises cyclosporin A, Lactipol S®, and Sunflower oil.

Another embodiment comprises cyclosporin A, Magrabar PGO®, and Sunflower oil.

Another embodiment comprises cyclosporin A, Mayco Base BFO®, and Sunflower oil.

Another embodiment comprises cyclosporin A, Methyl Linoleate®, and Sunflower oil.

Another embodiment comprises cyclosporin A, Pationic 122A®, and Sunflower oil.

Another embodiment comprises cyclosporin A, Pationic 138C®, and Sunflower oil.

Another embodiment comprises cyclosporin A, Pationic CSL®, and Sunflower oil.

Another embodiment comprises cyclosporin A, Pationic ISL®, and Sunflower oil.

Another embodiment comprises cyclosporin A, Pationic SBL®, and Sunflower oil.

Another embodiment comprises cyclosporin A, Pationic SSL®, and Sunflower oil.

Another embodiment comprises cyclosporin A, Ritasol®, and Sunflower oil.

Another embodiment comprises cyclosporin A, Tego Alkanol CS 20®, and Sunflower oil.

Another embodiment comprises cyclosporin A, Tego Alkanol L23 P®, and Sunflower oil.

Another embodiment comprises cyclosporin A, Tego Alkanol S2®, and Sunflower oil.

Another embodiment comprises cyclosporin A, Tego Alkanol S20 P®, and Sunflower oil.

Another embodiment comprises cyclosporin A, Triemulsifier 600 MS®, and Sunflower oil.

Another embodiment comprises cyclosporin A, a Glycerol Ester®, and Sunflower oil.

Another embodiment comprises cyclosporin A, Agro #9 Wint SBO®, and Sunflower oil.

Another embodiment comprises cyclosporin A, Ahcovel Base 700®, and Sunflower oil.

Another embodiment comprises cyclosporin A, Aldo HMS FG®, and Sunflower oil.

Another embodiment comprises cyclosporin A, Aldo MLD®, and Sunflower oil.

Another embodiment comprises cyclosporin A, Aldo MLD FG®, and Sunflower oil.

Another embodiment comprises cyclosporin A, Aldo MO FG®, and Sunflower oil.

Another embodiment comprises cyclosporin A, Aldo MS®, and Sunflower oil.

Another embodiment comprises cyclosporin A, Aldo MS FG®, and Sunflower oil.

Another embodiment comprises cyclosporin A, Aldo MS LG FG®, and Sunflower oil.

Another embodiment comprises cyclosporin A, Aldo MSD®, and Sunflower oil.

Another embodiment comprises cyclosporin A, Aldo MSD FG®, and Sunflower oil.

Another embodiment comprises cyclosporin A, Aldosperse O-20 FG®, and Sunflower oil.

Another embodiment comprises cyclosporin A, Aldosperse TS-20 FG®, and Sunflower oil.

Another embodiment comprises cyclosporin A, Aldosperse TS-40 FG®, and Sunflower oil.

Another embodiment comprises cyclosporin A, Arlacel 165®, and Sunflower oil.

Another embodiment comprises cyclosporin A, Arlacel 186®, and Sunflower oil.

Another embodiment comprises cyclosporin A, Capmul GMO®, and Sunflower oil.

Another embodiment comprises cyclosporin A, Capmul GMS®, and Sunflower oil.

Another embodiment comprises cyclosporin A, Caprol 3GO®, and Sunflower oil.

Another embodiment comprises cyclosporin A, Caprol 3GVS®, and Sunflower oil.

Another embodiment comprises cyclosporin A, Caprol 6G2S®, and Sunflower oil.

Another embodiment comprises cyclosporin A, Caprol 10G40®, and Sunflower oil.

Another embodiment comprises cyclosporin A, Caprol 10G100®, and Sunflower oil.

Another embodiment comprises cyclosporin A, Caprol ET®, and Sunflower oil.

Another embodiment comprises cyclosporin A, Caprol PGE860®, and Sunflower oil.

Another embodiment comprises cyclosporin A, Cerasynt 945®, and Sunflower oil.

Another embodiment comprises cyclosporin A, Cerasynt GMS®, and Sunflower oil.

Another embodiment comprises cyclosporin A, Cerasynt Q®, and Sunflower oil.

Another embodiment comprises cyclosporin A, Cerasynt SD®, and Sunflower oil.

Another embodiment comprises cyclosporin A, Cerasynt WM®, and Sunflower oil.

Another embodiment comprises cyclosporin A, Chemsperse 14®, and Sunflower oil.

Another embodiment comprises cyclosporin A, Cremophor GO-32®, and Sunflower oil.

Another embodiment comprises cyclosporin A, Cremophor GS11®, and Sunflower oil.

Another embodiment comprises cyclosporin A, Cremophor GS-32®, and Sunflower oil.

Another embodiment comprises cyclosporin A, Cutina KD-16®, and Sunflower oil.

Another embodiment comprises cyclosporin A, Dehymuls PGPH®, and Sunflower oil.

Another embodiment comprises cyclosporin A, Dermol DGDIS®, and Sunflower oil.

Another embodiment comprises cyclosporin A, Dermol DGMIS®, and Sunflower oil.

Another embodiment comprises cyclosporin A, Dermol G-76®, and Sunflower oil.

Another embodiment comprises cyclosporin A, Dermol G-7DI®, and Sunflower oil.

Another embodiment comprises cyclosporin A, Dermol NGDI®, and Sunflower oil.

Another embodiment comprises cyclosporin A, Dermolan GLH®, and Sunflower oil.

Another embodiment comprises cyclosporin A, Drewmulse GMO®, and Sunflower oil.

Another embodiment comprises cyclosporin A, Drewpol 3-5-M®, and Sunflower oil.

Another embodiment comprises cyclosporin A, Durlac 100 W®, and Sunflower oil.

Another embodiment comprises cyclosporin A, Dur-Lo®, and Sunflower oil.

Another embodiment comprises cyclosporin A, Dynasan 118®, and Sunflower oil.

Another embodiment comprises cyclosporin A, EC-25®, and Sunflower oil.

Another embodiment comprises cyclosporin A, EM 40®, and Sunflower oil.

Another embodiment comprises cyclosporin A, Emerest 2400®, and Sunflower oil.

Another embodiment comprises cyclosporin A, Emerest 2452®, and Sunflower oil.

Another embodiment comprises cyclosporin A, Empilan G-26®, and Sunflower oil.

Another embodiment comprises cyclosporin A, Genapol TSM®, and Sunflower oil.

Another embodiment comprises cyclosporin A, Hostacerin DGI®, and Sunflower oil.

Another embodiment comprises cyclosporin A, Hostacerin DGL®, and Sunflower oil.

Another embodiment comprises cyclosporin A, Hostacerin DGMS®, and Sunflower oil.

Another embodiment comprises cyclosporin A, Hostacerin DGSB®, and Sunflower oil.

Another embodiment comprises cyclosporin A, Ice No. 2®, and Sunflower oil.

Another embodiment comprises cyclosporin A, Imwitor 742®, and Sunflower oil.

Another embodiment comprises cyclosporin A, Imwitor 780 K®, and Sunflower oil.

Mother embodiment comprises cyclosporin A, Imwitor 960 Flakes®, and Sunflower oil.

Another embodiment comprises cyclosporin A, Isolan GI 34®, and Sunflower oil.

Another embodiment comprises cyclosporin A, Isolan GO 33®, and Sunflower oil.

Another embodiment comprises cyclosporin A, Kemester 1000®, and Sunflower oil.

Another embodiment comprises cyclosporin A, Kemester 2000®, and Sunflower oil.

Another embodiment comprises cyclosporin A, Kemester 2000®, and Sunflower oil.

Another embodiment comprises cyclosporin A, Kemester 6000SE®, and Sunflower oil.

Another embodiment comprises cyclosporin A, Lamecreme DGE 18®, and Sunflower oil.

Another embodiment comprises cyclosporin A, Lexemul 515®, and Sunflower oil.

Another embodiment comprises cyclosporin A, Lexemul 561®, and Sunflower oil.

Another embodiment comprises cyclosporin A, Lexemul AR®, and Sunflower oil.

Another embodiment comprises cyclosporin A, Lexemul AS®, and Sunflower oil.

Another embodiment comprises cyclosporin A, Lexemul GDL®, and Sunflower oil.

Another embodiment comprises cyclosporin A, Lexemul T®, and Sunflower oil.

Another embodiment comprises cyclosporin A, Lipomulse 165®, and Sunflower oil.

Another embodiment comprises cyclosporin A, Lumulse GML K®, and Sunflower oil.

Another embodiment comprises cyclosporin A, Lumulse GMO K®, and Sunflower oil.

Another embodiment comprises cyclosporin A, Lumulse GMR K®, and Sunflower oil.

Another embodiment comprises cyclosporin A, Lumulse GMT K®, and Sunflower oil.

Another embodiment comprises cyclosporin A, Magrabar GMC®, and Sunflower oil.

Another embodiment comprises cyclosporin A, Magrabar GMO-CK®, and Sunflower oil.

Another embodiment comprises cyclosporin A, Magrabar MDG-5050®, and Sunflower oil.

Another embodiment comprises cyclosporin A, Magrabar PGO-315®, and Sunflower oil.

Another embodiment comprises cyclosporin A, Magrabar PGO-1010®, and Sunflower oil.

Another embodiment comprises cyclosporin A, Mazol 300K®, and Sunflower oil.

Another embodiment comprises cyclosporin A, Mazol GMO-K®, and Sunflower oil.

Another embodiment comprises cyclosporin A, Mazol GMS-K®, and Sunflower oil.

Another embodiment comprises cyclosporin A, Mazol PG031-K®, and Sunflower oil.

Another embodiment comprises cyclosporin A, Miglyol 812®, and Sunflower oil.

Another embodiment comprises cyclosporin A, Norfox 165C®, and Sunflower oil.

Another embodiment comprises cyclosporin A, Schercemol GMIS®, and Sunflower oil.

Another embodiment comprises cyclosporin A, Tegin®, and Sunflower oil.

Another embodiment comprises cyclosporin A, Tegin 4100 Pellets®, and Sunflower oil.

Another embodiment comprises cyclosporin A, Tegin M Pellets®, and Sunflower oil.

Another embodiment comprises cyclosporin A, Tegin OV®, and Sunflower oil.

Another embodiment comprises cyclosporin A, Teginacid H®, and Sunflower oil.

Another embodiment comprises cyclosporin A, Tego-Cosmo P813®, and Sunflower oil.

Another embodiment comprises cyclosporin A, Wickenol 535®, and Sunflower oil.

Another embodiment comprises cyclosporin A, Witconol 14®, and Sunflower oil.

Another embodiment comprises cyclosporin A, Witconol 14®, and Sunflower oil.

Another embodiment comprises cyclosporin A, Witconol 14F®, and Sunflower oil.

Another embodiment comprises cyclosporin A, Witconol 18L®, and Sunflower oil.

Another embodiment comprises cyclosporin A, Witconol GOT®, and Sunflower oil.

Another embodiment comprises cyclosporin A, Witconol MST®, and Sunflower oil.

Another embodiment comprises cyclosporin A, Witconol RHT®, and Sunflower oil.

Another embodiment comprises cyclosporin A, a Glycol Ester, and Sunflower oil.

Another embodiment comprises cyclosporin A, Alkamuls 600 DO®, and Sunflower oil.

Another embodiment comprises cyclosporin A, Alkamuls SEG®, and Sunflower oil.

Another embodiment comprises cyclosporin A, Atlas EM-2®, and Sunflower oil.

Another embodiment comprises cyclosporin A, Cerasynt IP®, and Sunflower oil.

Another embodiment comprises cyclosporin A, Cerasynt M®, and Sunflower oil.

Another embodiment comprises cyclosporin A, Cerasynt MN®, and Sunflower oil.

Another embodiment comprises cyclosporin A, Cerasynt PA®, and Sunflower oil.

Another embodiment comprises cyclosporin A, Chemsperse EGDS®, and Sunflower oil.

Another embodiment comprises cyclosporin A, Chemsperse EGMS®, and Sunflower oil.

Another embodiment comprises cyclosporin A, Colonial Monolaurin®, and Sunflower oil.

Another embodiment comprises cyclosporin A, DeMuls SGE-95®, and Sunflower oil.

Another embodiment comprises cyclosporin A, Eccoterge 200®, and Sunflower oil.

Another embodiment comprises cyclosporin A, Emerest 2380®, and Sunflower oil.

Another embodiment comprises cyclosporin A, Ethox 2610®, and Sunflower oil.

Another embodiment comprises cyclosporin A, Ethox DO-9®, and Sunflower oil.

Another embodiment comprises cyclosporin A, Ethox DO-14®, and Sunflower oil.

Another embodiment comprises cyclosporin A, Ethox SO-9®, and Sunflower oil.

Another embodiment comprises cyclosporin A, Fizul MD-318®, and Sunflower oil.

Another embodiment comprises cyclosporin A, Genapol EGDS-VHP®, and Sunflower oil.

Another embodiment comprises cyclosporin A, Genapol TS Powder®, and Sunflower oil.

Another embodiment comprises cyclosporin A, Hostacerin WO®, and Sunflower oil.

Another embodiment comprises cyclosporin A, Inversol 140®, and Sunflower oil.

Another embodiment comprises cyclosporin A, Kemester 104®, and Sunflower oil.

Another embodiment comprises cyclosporin A, Kemester 205®, and Sunflower oil.

Another embodiment comprises cyclosporin A, Kemester 226®, and Sunflower oil.

Another embodiment comprises cyclosporin A, Kemester 5221 SE®, and Sunflower oil.

Another embodiment comprises cyclosporin A, Kemester EGDS®, and Sunflower oil.

Another embodiment comprises cyclosporin A, Lexemul EGDS®, and Sunflower oil.

Another embodiment comprises cyclosporin A, Lexemul EGMS®, and Sunflower oil.

Another embodiment comprises cyclosporin A, Lexemul P®, and Sunflower oil.

Another embodiment comprises cyclosporin A, Lipo DGLS®, and Sunflower oil.

Another embodiment comprises cyclosporin A, Lipo EGDS®, and Sunflower oil.

Another embodiment comprises cyclosporin A, Lipo PGMS®, and Sunflower oil.

Another embodiment comprises cyclosporin A, Liposorb S-4®, and Sunflower oil.

Another embodiment comprises cyclosporin A, Liposorb TO-20®, and Sunflower oil.

Another embodiment comprises cyclosporin A, Lumulse PGO®, and Sunflower oil.

Another embodiment comprises cyclosporin A, Mackester EGDS®, and Sunflower oil.

Another embodiment comprises cyclosporin A, Mackester EGMS®, and Sunflower oil.

Another embodiment comprises cyclosporin A, Mackester GSTP®, and Sunflower oil.

Another embodiment comprises cyclosporin A, Mackester Series®, and Sunflower oil.

Another embodiment comprises cyclosporin A, Magrabar PDG-50®, and Sunflower oil.

Another embodiment comprises cyclosporin A, Mapeg 6000 DS®, and Sunflower oil.

Another embodiment comprises cyclosporin A, Marlowet 4702®, and Sunflower oil.

Another embodiment comprises cyclosporin A, Monalube 305®, and Sunflower oil.

Another embodiment comprises cyclosporin A, Monalube 310®, and Sunflower oil.

Another embodiment comprises cyclosporin A, Monalube 315®, and Sunflower oil.

Another embodiment comprises cyclosporin A, Monalube 320®, and Sunflower oil.

Another embodiment comprises cyclosporin A, Monalube 325®, and Sunflower oil.

Another embodiment comprises cyclosporin A, Monalube 330®, and Sunflower oil.

Another embodiment comprises cyclosporin A, Naturechem PGHS®, and Sunflower oil.

Another embodiment comprises cyclosporin A, Polycastorol PLO-840®, and Sunflower oil.

Another embodiment comprises cyclosporin A, Polytex 10M®, and Sunflower oil.

Another embodiment comprises cyclosporin A, Ritasynt IP®, and Sunflower oil.

Another embodiment comprises cyclosporin A, Ross Chem PEG 600 DT®, and Sunflower oil.

Another embodiment comprises cyclosporin A, Schercemol PGMS®, and Sunflower oil.

Another embodiment comprises cyclosporin A, Sponto H-44C®, and Sunflower oil.

Another embodiment comprises cyclosporin A, Tegin G®, and Sunflower oil.

Another embodiment comprises cyclosporin A, Witbreak DGE-182®, and Sunflower oil.

Another embodiment comprises cyclosporin A, Witbreak DGE-182®, and Sunflower oil.

Another embodiment comprises cyclosporin A, Witbreak DRA-21®, and Sunflower oil.

Another embodiment comprises cyclosporin A, Witbreak DRA-21®, and Sunflower oil.

Another embodiment comprises cyclosporin A, Witbreak DRA-50®, and Sunflower oil.

Another embodiment comprises cyclosporin A, Witbreak DRA-50®, and Sunflower oil.

Another embodiment comprises cyclosporin A, Witconol F46-46®, and Sunflower oil.

Another embodiment comprises cyclosporin A, Witconol H-32®, and Sunflower oil.

Another embodiment comprises cyclosporin A, Witconol H-33®, and Sunflower oil.

Another embodiment comprises cyclosporin A, Witconol H-35A®, and Sunflower oil.

Another embodiment comprises cyclosporin A, Witconol RHP®, and Sunflower oil.

Another embodiment comprises cyclosporin A, a Lanolin-based Derivative, and Sunflower oil.

Another embodiment comprises cyclosporin A, Amerchol CAB®, and Sunflower oil.

Another embodiment comprises cyclosporin A, Amerchol L-101®, and Sunflower oil.

Another embodiment comprises cyclosporin A, Amerlate LFA-LO®, and Sunflower oil.

Another embodiment comprises cyclosporin A, Amerlate P®, and Sunflower oil.

Another embodiment comprises cyclosporin A, Barre Common Degras®, and Sunflower oil.

Another embodiment comprises cyclosporin A, Cholesterol NF®, and Sunflower oil.

Another embodiment comprises cyclosporin A, Crodalan AWS®, and Sunflower oil.

Another embodiment comprises cyclosporin A, Crodalan LA®, and Sunflower oil.

Another embodiment comprises cyclosporin A, Emery 1650®, and Sunflower oil.

Another embodiment comprises cyclosporin A, Emery 1650®, and Sunflower oil.

Another embodiment comprises cyclosporin A, Emery 1740®, and Sunflower oil.

Another embodiment comprises cyclosporin A, Emery 1740®, and Sunflower oil.

Another embodiment comprises cyclosporin A, Forlan 500®, and Sunflower oil.

Another embodiment comprises cyclosporin A, Forlan L®, and Sunflower oil.

Another embodiment comprises cyclosporin A, Laneto 50®, and Sunflower oil.

Another embodiment comprises cyclosporin A, Laneto 100®, and Sunflower oil.

Another embodiment comprises cyclosporin A, Laneto AWS®, and Sunflower oil.

Another embodiment comprises cyclosporin A, Lanfrax 1776®, and Sunflower oil.

Another embodiment comprises cyclosporin A, Lanfrax 1776®, and Sunflower oil.

Another embodiment comprises cyclosporin A, Lanogel 21®, and Sunflower oil.

Another embodiment comprises cyclosporin A, Lipolan®, and Sunflower oil.

Another embodiment comprises cyclosporin A, Lipolan 31®, and Sunflower oil.

Another embodiment comprises cyclosporin A, OHlan®, and Sunflower oil.

Another embodiment comprises cyclosporin A, Polychol 5®, and Sunflower oil.

Another embodiment comprises cyclosporin A, Polychol 15®, and Sunflower oil.

Another embodiment comprises cyclosporin A, Ritacetyl®, and Sunflower oil.

Another embodiment comprises cyclosporin A, Ritachol®, and Sunflower oil.

Another embodiment comprises cyclosporin A, Ritahydrox®, and Sunflower oil.

Another embodiment comprises cyclosporin A, Ritalafa®, and Sunflower oil.

Another embodiment comprises cyclosporin A, Ritalan®, and Sunflower oil.

Another embodiment comprises cyclosporin A, Ritalan AWS®, and Sunflower oil.

Another embodiment comprises cyclosporin A, Ritalan C®, and Sunflower oil.

Another embodiment comprises cyclosporin A, Ritawax®, and Sunflower oil.

Another embodiment comprises cyclosporin A, Ritawax AEO®, and Sunflower oil.

Another embodiment comprises cyclosporin A, Ritawax ALA®, and Sunflower oil.

Another embodiment comprises cyclosporin A, Solan/Solan 50/Super Solan®, and Sunflower oil.

Another embodiment comprises cyclosporin A, Super Hartolan/Hartolan®, and Sunflower oil.

Another embodiment comprises cyclosporin A, Supersat AWS-4®, and Sunflower oil.

Another embodiment comprises cyclosporin A, Supersat AWS-24®, and Sunflower oil.

Another embodiment comprises cyclosporin A, a Lecithin or Lecithin Derivative, and Sunflower oil.

Another embodiment comprises cyclosporin A, Alcolec®, and Sunflower oil.

Another embodiment comprises cyclosporin A, Lecithin®, and Sunflower oil.

Another embodiment comprises cyclosporin A, Lexin K®, and Sunflower oil.

Another embodiment comprises cyclosporin A, Natipide®, and Sunflower oil.

Another embodiment comprises cyclosporin A, a Lignin or Lignin Derivative, and Sunflower oil.

Another embodiment comprises cyclosporin A, Diwatex XP 9®, and Sunflower oil.

Another embodiment comprises cyclosporin A, Dynasperse LCD®, and Sunflower oil.

Another embodiment comprises cyclosporin A, Indulin SAL®, and Sunflower oil.

Another embodiment comprises cyclosporin A, Indulin W-1®,and Sunflower oil.

Another embodiment comprises cyclosporin A, Indulin W-5®, and Sunflower oil.

Another embodiment comprises cyclosporin A, Lignosol FTA®, and Sunflower oil.

Another embodiment comprises cyclosporin A, Lignosol SFX-65®, and Sunflower oil.

Another embodiment comprises cyclosporin A, Marasperse 52 CP®, and Sunflower oil.

Another embodiment comprises cyclosporin A, Marasperse AG®, and Sunflower oil.

Another embodiment comprises cyclosporin A, Marasperse CBOS-4®,and Sunflower oil.

Another embodiment comprises cyclosporin A, Ufoxane 2®, and Sunflower oil.

Another embodiment comprises cyclosporin A, a Methyl Ester, and Sunflower oil.

Another embodiment comprises cyclosporin A, E.B. Cleaner AK®, and Sunflower oil.

Another embodiment comprises cyclosporin A, Oleocal ME-70®, and Sunflower oil.

Another embodiment comprises cyclosporin A, Trade Name Company®, and Sunflower oil.

Another embodiment comprises cyclosporin A, Methyl Esters (cont'd)®, and Sunflower oil.

Another embodiment comprises cyclosporin A, Oleocal ME-92®, and Sunflower oil.

Another embodiment comprises cyclosporin A, Oleocal ME-112, and Sunflower oil.

Another embodiment comprises cyclosporin A, Oleocal ME-130®, and Sunflower oil.

Another embodiment comprises cyclosporin A, a Monoglyceride or a Derivative thereof, and Sunflower oil.

Another embodiment comprises cyclosporin A, Dynacet 211®, and Sunflower oil.

Another embodiment comprises cyclosporin A, Hetsorb S-20®, and Sunflower oil.

Another embodiment comprises cyclosporin A, Imwitor 191®, and Sunflower oil.

Another embodiment comprises cyclosporin A, Imwitor 370®, and Sunflower oil.

Another embodiment comprises cyclosporin A, Imwitor 375®, and Sunflower oil.

Another embodiment comprises cyclosporin A, Imwitor 900®, and Sunflower oil.

Another embodiment comprises cyclosporin A, Imwitor 945®, and Sunflower oil.

Another embodiment comprises cyclosporin A, Imwitor 2020®, and Sunflower oil.

Another embodiment comprises cyclosporin A, Kemester 5500®, and Sunflower oil.

Another embodiment comprises cyclosporin A, Kemester 6000®, and Sunflower oil.

Another embodiment comprises cyclosporin A, Magrabar GMC®, and Sunflower oil.

Another embodiment comprises cyclosporin A, Magrabar GMO-CK®, and Sunflower oil.

Another embodiment comprises cyclosporin A, Magrabar GPC-10®, and Sunflower oil.

Another embodiment comprises cyclosporin A, Magrabar MDG-5050®, and Sunflower oil.

Another embodiment comprises cyclosporin A, Monalube 335®, and Sunflower oil.

Another embodiment comprises cyclosporin A, Monoglycerides Glyceryl Monestearate Archer Daniels Midland Company®, and Sunflower oil.

Another embodiment comprises cyclosporin A, Rita GMS®, and Sunflower oil.

Another embodiment comprises cyclosporin A, Ritamulse SCG®, and Sunflower oil.

Another embodiment comprises cyclosporin A, Softigen 701®, and Sunflower oil.

Another embodiment comprises cyclosporin A, Tally 100 Plus®, and Sunflower oil.

Another embodiment comprises cyclosporin A, a Polyethylene Glycol, and Sunflower oil.

Another embodiment cormiprises cyclosporin A, Emulgade PL 68/50®, and Sunflower oil.

Another embodiment comprises cyclosporin A, Lumulse PEG®, and Sunflower oil.

Another embodiment comprises cyclosporin A, Rhodasun PEG-400®, and Sunflower oil.

Another embodiment comprises cyclosporin A, Rhodasurt PEG-600®, and Sunflower oil.

Another embodiment comprises cyclosporin A, Witconol PEG-400®, and Sunflower oil.

Another embodiment comprises cyclosporin A, a Polymeric Surfactant, and Sunflower oil.

Another embodiment comprises cyclosporin A, Acritamer PNC-EG®, and Sunflower oil.

Another embodiment comprises cyclosporin A, Ag-Rho DEP-775®, and Sunflower oil.

Another embodiment comprises cyclosporin A, APG 325N Glycoside®, and Sunflower oil.

Another embodiment comprises cyclosporin A, Aristoflex AVC®, and Sunflower oil.

Another embodiment comprises cyclosporin A, Aristoflex HMB®, and Sunflower oil.

Another embodiment comprises cyclosporin A, Burco NPS-225®, and Sunflower oil.

Another embodiment comprises cyclosporin A, Burco NPS-816®, and Sunflower oil.

Another embodiment comprises cyclosporin A, Chemccinate 5603®, and Sunflower oil.

Another embodiment comprises cyclosporin A, Cosmedia Guar C-261N®, and Sunflower oil.

Another embodiment comprises cyclosporin A, Gantrez S-95®, and Sunflower oil.

Another embodiment comprises cyclosporin A, Glucopon 220 UP®, and Sunflower oil.

Another embodiment comprises cyclosporin A, Glucopon 225 DK®, and Sunflower oil.

Another embodiment comprises cyclosporin A, Glucopon 425 N®, and Sunflower oil.

Another embodiment comprises cyclosporin A, Glucopon 600 UP®, and Sunflower oil.

Another embodiment comprises cyclosporin A, Glucopon 625 UP®, and Sunflower oil.

Another embodiment comprises cyclosporin A, Pemulen 1621®, and Sunflower oil.

Another embodiment comprises cyclosporin A, Pemulen 1622®, and Sunflower oil.

Another embodiment comprises cyclosporin A, Pemulen TR-1®, and Sunflower oil.

Another embodiment comprises cyclosporin A, Pemulen TR-2®, and Sunflower oil.

Another embodiment comprises cyclosporin A, Plantacare 818®, and Sunflower oil.

Another embodiment comprises cyclosporin A, Plantapon LGC Sorb®, and Sunflower oil.

Another embodiment comprises cyclosporin A, Plantaren 1200N®, and Sunflower oil.

Another embodiment comprises cyclosporin A, Plantaren 2000N®, and Sunflower oil.

Another embodiment comprises cyclosporin A, Viscolam AT 64®, and Sunflower oil.

Another embodiment comprises cyclosporin A, Viscolam AT 64P®, and Sunflower oil.

Another embodiment comprises cyclosporin A, Viscolam AT 100®, and Sunflower oil.

Another embodiment comprises cyclosporin A, Viscolam MAC 7®, and Sunflower oil.

Another embodiment comprises cyclosporin A, Viscolam SMC 20®, and Sunflower oil.

Another embodiment comprises cyclosporin A, Witbreak RTC-323®, and Sunflower oil.

Another embodiment comprises cyclosporin A, WSI 3700®, and Sunflower oil.

Another embodiment comprises cyclosporin A, a Propoxylated or Ethoxylated Fatty Acid, Alcohol, or Alkyl Phenol, and Sunflower oil.

Another embodiment comprises cyclosporin A, Antarox AA-60®, and Sunflower oil.

Another embodiment comprises cyclosporin A, Antarox LF-224®, and Sunflower oil.

Another embodiment comprises cyclosporin A, Burcomul DFE-45®, and Sunflower oil.

Another embodiment comprises cyclosporin A, Burcoterge LFE-1000®, and Sunflower oil.

Another embodiment comprises cyclosporin A, Chemal LF-25B®, and Sunflower oil.

Another embodiment comprises cyclosporin A, Chemal LF-40B®, and Sunflower oil.

Another embodiment comprises cyclosporin A, Dehypon LS-36®, and Sunflower oil.

Another embodiment comprises cyclosporin A, Dehypon LS-36®, and Sunflower oil.

Another embodiment comprises cyclosporin A, Dehypon LS-54®, and Sunflower oil.

Another embodiment comprises cyclosporin A, Dehypon LS-54®, and Sunflower oil.

Another embodiment comprises cyclosporin A, Delonic 100 VLF®, and Sunflower oil.

Another embodiment comprises cyclosporin A, Delonic LF-60 MOD®, and Sunflower oil.

Another embodiment comprises cyclosporin A, Empiderm B®, and Sunflower oil.

Another embodiment comprises cyclosporin A, Ethylan 1206®, and Sunflower oil.

Another embodiment comprises cyclosporin A, Ethylan NS-500K®, and Sunflower oil.

Another embodiment comprises cyclosporin A, Ethylan NS-500LQ®, and Sunflower oil.

Another embodiment comprises cyclosporin A, Genapol 1392®, and Sunflower oil.

Another embodiment comprises cyclosporin A, Genapol 2317®, and Sunflower oil.

Another embodiment comprises cyclosporin A, Genapol 26EP710®, and Sunflower oil.

Another embodiment comprises cyclosporin A, Genapol EP 1022®, and Sunflower oil.

Another embodiment comprises cyclosporin A, Genapol EP 1024®, and Sunflower oil.

Another embodiment comprises cyclosporin A, Genapol EP 6068®, and Sunflower oil.

Another embodiment comprises cyclosporin A, Genapol NP915®, and Sunflower oil.

Another embodiment comprises cyclosporin A, Kieralon MFB®, and Sunflower oil.

Another embodiment comprises cyclosporin A, Lumisolve CSA-80 V®, and Sunflower oil.

Another embodiment comprises cyclosporin A, Marlowet 5001®, and Sunflower oil.

Another embodiment comprises cyclosporin A, Marlox FK 64®, and Sunflower oil.

Another embodiment comprises cyclosporin A, Marlox MO 124®, and Sunflower oil.

Another embodiment comprises cyclosporin A, Marlox S 58®, and Sunflower oil.

Another embodiment comprises cyclosporin A, Nonatell 1003®, and Sunflower oil.

Another embodiment comprises cyclosporin A, Nonatell 1038®, and Sunflower oil.

Another embodiment comprises cyclosporin A, Nonatell 1052®, and Sunflower oil.

Another embodiment comprises cyclosporin A, Nonatell 1061®, and Sunflower oil.

Another embodiment comprises cyclosporin A, Nonatell 1075®, and Sunflower oil.

Another embodiment comprises cyclosporin A, Nonatell 1088®, and Sunflower oil.

Another embodiment comprises cyclosporin A, Nonatell 1123®, and Sunflower oil.

Another embodiment comprises cyclosporin A, Nonatell 1153®, and Sunflower oil.

Another embodiment comprises cyclosporin A, Nonatell 1161®, and Sunflower oil.

Another embodiment comprises cyclosporin A, Nonatell 1172®, and Sunflower oil.

Another embodiment comprises cyclosporin A, Nonatell 1181®, and Sunflower oil.

Another embodiment comprises cyclosporin A, Norfox 36®, and Sunflower oil.

Another embodiment comprises cyclosporin A, Procetyl AWS®, and Sunflower oil.

Another embodiment comprises cyclosporin A, Sandoxylate SX 412®, and Sunflower oil.

Another embodiment comprises cyclosporin A, Sandoxylate SX 418®, and Sunflower oil.

Another embodiment comprises cyclosporin A, Surfonic JL-80X®, and Sunflower oil.

Another embodiment comprises cyclosporin A, Surfonic JL-80X-B1®, and Sunflower oil.

Another embodiment comprises cyclosporin A, Surfonic L4-29X®, and Sunflower oil.

Another embodiment comprises cyclosporin A, Surfonic LF®, and Sunflower oil.

Another embodiment comprises cyclosporin A, T-Det A826®, and Sunflower oil.

Another embodiment comprises cyclosporin A, T-Det LF-416®, and Sunflower oil.

Another embodiment comprises cyclosporin A, Tergitol Min-Foam 1X®, and Sunflower oil.

Another embodiment comprises cyclosporin A, Tergitol Min-Foam 2X®, and Sunflower oil.

Another embodiment comprises cyclosporin A, Triton CF-21®, and Sunflower oil.

Another embodiment comprises cyclosporin A, Triton CF-76®, and Sunflower oil.

Another embodiment comprises cyclosporin A, Triton XL-80N®, and Sunflower oil.

Another embodiment comprises cyclosporin A, Witconol NS-98®, and Sunflower oil.

Another embodiment comprises cyclosporin A, Witconol NS-108LQ®, and Sunflower oil.

Another embodiment comprises cyclosporin A, Witconol NS-145®, and Sunflower oil.

Another embodiment comprises cyclosporin A, Witconol NS-179®, and Sunflower oil.

Another embodiment comprises cyclosporin A, a Protein-based Surfactant, and Sunflower oil.

Another embodiment comprises cyclosporin A, AminoFoam W®, and Sunflower oil.

Another embodiment comprises cyclosporin A, Amiter LGOD-2®, and Sunflower oil.

Another embodiment comprises cyclosporin A, Amiter LGS-2®, and Sunflower oil.

Another embodiment comprises cyclosporin A, Amiter LGS-5®, and Sunflower oil.

Another embodiment comprises cyclosporin A, Lamepon S®, and Sunflower oil.

Another embodiment comprises cyclosporin A, Lamepon S®, and Sunflower oil.

Another embodiment comprises cyclosporin A, Maypon 4C®, and Sunflower oil.

Another embodiment comprises cyclosporin A, May-Tein C®, and Sunflower oil.

Another embodiment comprises cyclosporin A, May-Tein CT®, and Sunflower oil.

Another embodiment comprises cyclosporin A, May-Tein KTS®, and Sunflower oil.

Another embodiment comprises cyclosporin A, May-Tein SY®, and Sunflower oil.

Another embodiment comprises cyclosporin A, Plantapon S®, and Sunflower oil.

Another embodiment comprises cyclosporin A, Proteol APL®, and Sunflower oil.

Another embodiment comprises cyclosporin A, Proteol OAT®, and Sunflower oil.

Another embodiment comprises cyclosporin A, Pyroter CPI-40®, and Sunflower oil.

Another embodiment comprises cyclosporin A, Pyroter GPI-25®, and Sunflower oil.

Another embodiment comprises cyclosporin A, Supro-Tein S®, and Sunflower oil.

Another embodiment comprises cyclosporin A, Supro-Tein V®, and Sunflower oil.

Another embodiment comprises cyclosporin A,® and Sunflower oil.

Another embodiment comprises cyclosporin A, a Sarcosine Derivative®, and Sunflower oil.

Another embodiment comprises cyclosporin A, Crodasinic LS-30®, and Sunflower oil.

Another embodiment comprises cyclosporin A, Vanseal CS®, and Sunflower oil.

Another embodiment comprises cyclosporin A, Vanseal LS®, and Sunflower oil.

Another embodiment comprises cyclosporin A, Vanseal MS®, and Sunflower oil.

Another embodiment comprises cyclosporin A, Vanseal NACS-30®, and Sunflower oil.

Another embodiment comprises cyclosporin A, Vanseal NALS-95®, and Sunflower oil.

Another embodiment comprises cyclosporin A, Vanseal OS®, and Sunflower oil.

Another embodiment comprises cyclosporin A,® and Sunflower oil.

Another embodiment comprises cyclosporin A, a Silicone-based Surfactant®, and Sunflower oil.

Another embodiment comprises cyclosporin A, Abil-B-9950®, and Sunflower oil.

Another embodiment comprises cyclosporin A, Abil Care 85®, and Sunflower oil.

Another embodiment comprises cyclosporin A, Abil EM 90®, and Sunflower oil.

Another embodiment comprises cyclosporin A, Abil EM 97®, and Sunflower oil.

Another embodiment comprises cyclosporin A, Abil WE-09®, and Sunflower oil.

Another embodiment comprises cyclosporin A, Dow Corning 1248 Fluid®, and Sunflower oil.

Another embodiment comprises cyclosporin A, Dow Corning 3225C®, and Sunflower oil.

Another embodiment comprises cyclosporin A, Dow Corning 5200®, and Sunflower oil.

Another embodiment comprises cyclosporin A, Dow Corning Q4-3667®, and Sunflower oil.

Another embodiment comprises cyclosporin A, Monasil PCA®, and Sunflower oil.

Another embodiment comprises cyclosporin A, Monasil PDM®, and Sunflower oil.

Another embodiment comprises cyclosporin A, Monasil PLN®, and Sunflower oil.

Another embodiment comprises cyclosporin A, Polyderm PPI-SI-WS®, and Sunflower oil.

Another embodiment comprises cyclosporin A, Troysol 380W®, and Sunflower oil.

Another embodiment comprises cyclosporin A, Troysol S366®, and Sunflower oil.

Another embodiment comprises cyclosporin A, a Sorbitan Derivative, and Sunflower oil.

Another embodiment comprises cyclosporin A, Alkamuls SML®, and Sunflower oil.

Another embodiment comprises cyclosporin A, Alkamuls SMO®, and Sunflower oil.

Another embodiment comprises cyclosporin A, Alkamuls STO®, and Sunflower oil.

Another embodiment comprises cyclosporin A, Arlacel 20®, and Sunflower oil.

Another embodiment comprises cyclosporin A, Arlacel 40®, and Sunflower oil.

Another embodiment comprises cyclosporin A, Arlacel 60®, and Sunflower oil.

Another embodiment comprises cyclosporin A, Arlacel 80®, and Sunflower oil.

Another embodiment comprises cyclosporin A, Arlacel C®, and Sunflower oil.

Another embodiment comprises cyclosporin A, Armul 21®, and Sunflower oil.

Another embodiment comprises cyclosporin A, Atlox 80®, and Sunflower oil.

Another embodiment comprises cyclosporin A, Atlox 847®, and Sunflower oil.

Another embodiment comprises cyclosporin A, Atlox 1045A®, and Sunflower oil.

Another embodiment comprises cyclosporin A, Canarcel 20®, and Sunflower oil.

Another embodiment comprises cyclosporin A, Canarcel 60®, and Sunflower oil.

Another embodiment comprises cyclosporin A, Canarcel 80®, and Sunflower oil.

Another embodiment comprises cyclosporin A, Canarcel TW 20®, and Sunflower oil.

Another embodiment comprises cyclosporin A, Canarcel TW 60®, and Sunflower oil.

Another embodiment comprises cyclosporin A, Canarcel TW 80®, and Sunflower oil.

Another embodiment comprises cyclosporin A, Coladet BSB-P®, and Sunflower oil.

Another embodiment comprises cyclosporin A, Customulse O-20®, and Sunflower oil.

Another embodiment comprises cyclosporin A, Dehymuls E®, and Sunflower oil.

Another embodiment comprises cyclosporin A, DeSotan SMO®, and Sunflower oil.

Another embodiment comprises cyclosporin A, DeSotan SMO-20®, and Sunflower oil.

Another embodiment comprises cyclosporin A, DeSotan SMT®, and Sunflower oil.

Another embodiment comprises cyclosporin A, DeSotan SMT-20®, and Sunflower oil.

Another embodiment comprises cyclosporin A, Durfax 60®, and Sunflower oil.

Another embodiment comprises cyclosporin A, Durfax 65®, and Sunflower oil.

Another embodiment comprises cyclosporin A, Durfax 80®, and Sunflower oil.

Another embodiment comprises cyclosporin A, Durtan 60®, and Sunflower oil.

Another embodiment comprises cyclosporin A, Durtan 65®, and Sunflower oil.

Another embodiment comprises cyclosporin A, Liposorb L®, and Sunflower oil.

Another embodiment comprises cyclosporin A, Liposorb L-10®, and Sunflower oil.

Another embodiment comprises cyclosporin A, Liposorb L-20®, and Sunflower oil.

Another embodiment comprises cyclosporin A, Liposorb O®, and Sunflower oil.

Another embodiment comprises cyclosporin A, Liposorb O-20®, and Sunflower oil.

Another embodiment comprises cyclosporin A, Liposorb P®, and Sunflower oil.

Another embodiment comprises cyclosporin A, Liposorb P-20®, and Sunflower oil.

Another embodiment comprises cyclosporin A, Liposorb S®, and Sunflower oil.

Another embodiment comprises cyclosporin A, Liposorb S-20®, and Sunflower oil.

Another embodiment comprises cyclosporin A, Liposorb SQO®, and Sunflower oil.

Another embodiment comprises cyclosporin A, Liposorb TO®, and Sunflower oil.

Another embodiment comprises cyclosporin A, Liposorb TS®, and Sunflower oil.

Another embodiment comprises cyclosporin A, Liposorb TS-20®, and Sunflower oil.

Another embodiment comprises cyclosporin A, Lumisorb PS®, and Sunflower oil.

Another embodiment comprises cyclosporin A, Lumisorb SMO (T)®, and Sunflower oil.

Another embodiment comprises cyclosporin A, Lumisorb SMS K®I, and Sunflower oil.

Another embodiment comprises cyclosporin A, Lumisorb SSO®, and Sunflower oil.

Another embodiment comprises cyclosporin A, Lumisorb STS K®, and Sunflower oil.

Another embodiment comprises cyclosporin A, Lumisorb STT®, and Sunflower oil.

Another embodiment comprises cyclosporin A, Magrabar SMO®, and Sunflower oil.

Another embodiment comprises cyclosporin A, Magrabar SMO-VEG®, and Sunflower oil.

Another embodiment comprises cyclosporin A, Magrabar SMT®, and Sunflower oil.

Another embodiment comprises cyclosporin A, Magrabar STO®, and Sunflower oil.

Another embodiment comprises cyclosporin A, Miracare BC-27®, and Sunflower oil.

Another embodiment comprises cyclosporin A, Ritabate 20®, and Sunflower oil.

Another embodiment comprises cyclosporin A, Ritabate 40®, and Sunflower oil.

Another embodiment comprises cyclosporin A, Ritabate 60®, and Sunflower oil.

Another embodiment comprises cyclosporin A, Ritabate 80®, and Sunflower oil.

Another embodiment comprises cyclosporin A, T-Maz®, and Sunflower oil.

Another embodiment comprises cyclosporin A, Tego SML®, and Sunflower oil.

Another embodiment comprises cyclosporin A, Tego SML 20®, and Sunflower oil.

Another embodiment comprises cyclosporin A, Tego SMO 80 V®, and Sunflower oil.

Another embodiment comprises cyclosporin A, Tego SMO V®, and Sunflower oil.

Another embodiment comprises cyclosporin A, Tego SMS®, and Sunflower oil.

Another embodiment comprises cyclosporin A, Tego STO V®, and Sunflower oil.

Another embodiment comprises cyclosporin A, Tween 21®, and Sunflower oil.

Another embodiment comprises cyclosporin A, Tween 40®, and Sunflower oil.

Another embodiment comprises cyclosporin A, Tween 60®, and Sunflower oil.

Another embodiment comprises cyclosporin A, Tween 60 K®, and Sunflower oil.

Another embodiment comprises cyclosporin A, Tween 61®, and Sunflower oil.

Another embodiment comprises cyclosporin A, Tween 65®, and Sunflower oil.

Another embodiment comprises cyclosporin A, Tween 80®, and Sunflower oil.

Another embodiment comprises cyclosporin A, Tween 80 K®, and Sunflower oil.

Another embodiment comprises cyclosporin A, Tween 81®, and Sunflower oil.

Another embodiment comprises cyclosporin A, Tween 85®, and Sunflower oil.

Another embodiment comprises cyclosporin A, a Sucrose or Glucose Ester, or Derivative thereof, and Sunflower oil.

Another embodiment comprises cyclosporin A, DeSulf GOS-P-60WCG®, and Sunflower oil.

Another embodiment comprises cyclosporin A, Glucam E-20 Distearate®, and Sunflower oil.

Another embodiment comprises cyclosporin A, Glucamate DOE-120®, and Sunflower oil.

Another embodiment comprises cyclosporin A, Glucamate SSE-20®, and Sunflower oil.

Another embodiment comprises cyclosporin A, Glucate DO®, and Sunflower oil.

Another embodiment comprises cyclosporin A, Glucate SS®, and Sunflower oil.

Another embodiment comprises cyclosporin A, Glucopon 425 UP®, and Sunflower oil.

Another embodiment comprises cyclosporin A, Isolan IS®, and Sunflower oil.

Another embodiment comprises cyclosporin A, Mazon 40®, and Sunflower oil.

Another embodiment comprises cyclosporin A, Montanov 82®, and Sunflower oil.

Another embodiment comprises cyclosporin A, Montanov 202®, and Sunflower oil.

Another embodiment comprises cyclosporin A, Montanov S®, and Sunflower oil.

Another embodiment comprises cyclosporin A, Rheozan®, and Sunflower oil.

Another embodiment comprises cyclosporin A, Simulsol AS 48®, and Sunflower oil.

Another embodiment comprises cyclosporin A, Simulsol SL 4®, and Sunflower oil.

Another embodiment comprises cyclosporin A, Simulsol SL 10®, and Sunflower oil.

Another embodiment comprises cyclosporin A, Simulsol SL 11W®, and Sunflower oil.

Another embodiment comprises cyclosporin A, Simulsol SL 55®, and Sunflower oil.

Another embodiment comprises cyclosporin A, Suga Nate 100 and 160®, and Sunflower oil.

Another embodiment comprises cyclosporin A, Tego Care 450®, and Sunflower oil.

Another embodiment comprises cyclosporin A, Tego Care CG 90®, and Sunflower oil.

Another embodiment comprises cyclosporin A, Tego Care PS®, and Sunflower oil.

Another embodiment comprises cyclosporin A, Tegosoft PSE 141 G®, and Sunflower oil.

Another embodiment comprises cyclosporin A, Tegotens G 826®, and Sunflower oil.

Another embodiment comprises cyclosporin A, Triton BG-10 (70%)®, and Sunflower oil.

Another embodiment comprises cyclosporin A, Triton CG-110 (60%)®, and Sunflower oil.

Another embodiment comprises cyclosporin A, Wickenol 545®, and Sunflower oil.

Another embodiment comprises cyclosporin A, an alcohol, and Eucalyptus oil.

Another embodiment comprises cyclosporin A, Diglycerol®, and Eucalyptus oil.

Another embodiment comprises cyclosporin A, Hetoxide GT-80®, and Eucalyptus oil.

Another embodiment comprises cyclosporin A, Lexemul BEO®, and Eucalyptus oil.

Another embodiment comprises cyclosporin A, Polyglycerol-3®, and Eucalyptus oil.

Another embodiment comprises cyclosporin A, Redicote E Series®, and Eucalyptus oil.

Another embodiment comprises cyclosporin A, Simulsol OX 1005L®, and Eucalyptus oil.

Another embodiment comprises cyclosporin A, Stanfax 567®, and Eucalyptus oil.

Another embodiment comprises cyclosporin A, TA-1618®, and Eucalyptus oil.

Another embodiment comprises cyclosporin A, Witconol H-31A®, and Eucalyptus oil.

Another embodiment comprises cyclosporin A, an amine oxide, and Eucalyptus oil.

Another embodiment comprises cyclosporin A, AO-405®, and Eucalyptus oil.

Another embodiment comprises cyclosporin A, AO-455®, and Eucalyptus oil.

Another embodiment comprises cyclosporin A, AO 728 Special®, and Eucalyptus oil.

Another embodiment comprises cyclosporin A, Barlox 12®, and Eucalyptus oil.

Another embodiment comprises cyclosporin A, Barlox 14®, and Eucalyptus oil.

Another embodiment comprises cyclosporin A, Burcoxide Lo®, and Eucalyptus oil.

Another embodiment comprises cyclosporin A, Caloxamine LO®, and Eucalyptus oil.

Another embodiment comprises cyclosporin A, Chemoxide CAW®, and Eucalyptus oil.

Another embodiment comprises cyclosporin A, Chemoxide LM-30®, and Eucalyptus oil.

Another embodiment comprises cyclosporin A, Chemoxide LO®, and Eucalyptus oil.

Another embodiment comprises cyclosporin A, Chemoxide MO®, and Eucalyptus oil.

Another embodiment comprises cyclosporin A, Colalux CAO-35®, and Eucalyptus oil.

Another embodiment comprises cyclosporin A, Colalux LO®, and Eucalyptus oil.

Another embodiment comprises cyclosporin A, DeMox CAPO®, and Eucalyptus oil.

Another embodiment comprises cyclosporin A, DeMox CSG-30®, and Eucalyptus oil.

Another embodiment comprises cyclosporin A, DeMox LAO®, and Eucalyptus oil.

Another embodiment comprises cyclosporin A, Emcol L®, and Eucalyptus oil.

Another embodiment comprises cyclosporin A, Empigen OB®, and Eucalyptus oil.

Another embodiment comprises cyclosporin A, Empigen OS/A®, and Eucalyptus oil.

Another embodiment comprises cyclosporin A, Foamox CDO®, and Eucalyptus oil.

Another embodiment comprises cyclosporin A, Foamox DMM®, and Eucalyptus oil.

Another embodiment comprises cyclosporin A, Foamox DMS®, and Eucalyptus oil.

Another embodiment comprises cyclosporin A, Genaminox KC®, and Eucalyptus oil.

Another embodiment comprises cyclosporin A, Genaminox LA®, and Eucalyptus oil.

Another embodiment comprises cyclosporin A, Hartofoam SAO®, and Eucalyptus oil.

Another embodiment comprises cyclosporin A, Hartox DMCD®, and Eucalyptus oil.

Another embodiment comprises cyclosporin A, Lipowax DAT®, and Eucalyptus oil.

Another embodiment comprises cyclosporin A, Lipowax PB Pastilles®, and Eucalyptus oil.

Another embodiment comprises cyclosporin A, Mackamine C8®, and Eucalyptus oil.

Another embodiment comprises cyclosporin A, Mackamine C10®, and Eucalyptus oil.

Another embodiment comprises cyclosporin A, Mackamine C14®, and Eucalyptus oil.

Another embodiment comprises cyclosporin A, Mackamine CAO®, and Eucalyptus oil.

Another embodiment comprises cyclosporin A, Mackamine CO®, and Eucalyptus oil.

Another embodiment comprises cyclosporin A, Mackamine LO®, and Eucalyptus oil.

Another embodiment comprises cyclosporin A, Mackamine O2®, and Eucalyptus oil.

Another embodiment comprises cyclosporin A, Mackamine SAO®, and Eucalyptus oil.

Another embodiment comprises cyclosporin A, Mackamine SO®, and Eucalyptus oil.

Another embodiment comprises cyclosporin A, Mazox KCAO®, and Eucalyptus oil.

Another embodiment comprises cyclosporin A, Monalac MO®, and Eucalyptus oil.

Another embodiment comprises cyclosporin A, Nonox LDA®, and Eucalyptus oil.

Another embodiment comprises cyclosporin A, Rhodamox LO®, and Eucalyptus oil.

Another embodiment comprises cyclosporin A, Schercamox C-AA®, and Eucalyptus oil.

Another embodiment comprises cyclosporin A, Schercamox DMA®, and Eucalyptus oil.

Another embodiment comprises cyclosporin A, Schercamox DML®, and Eucalyptus oil.

Another embodiment comprises cyclosporin A, Schercamox DMM®, and Eucalyptus oil.

Another embodiment comprises cyclosporin A, Schercamox DMS®, and Eucalyptus oil.

Another embodiment comprises cyclosporin A, Tegotens DO®, and Eucalyptus oil.

Another embodiment comprises cyclosporin A, Tomah AO-14-2®, and Eucalyptus oil.

Another embodiment comprises cyclosporin A, Triaminox CDO®, and Eucalyptus oil.

Another embodiment comprises cyclosporin A, a block polymer, and Eucalyptus oil.

Another embodiment comprises cyclosporin A, AL 2070®, and Eucalyptus oil.

Another embodiment comprises cyclosporin A, Antarox 17-R-2®, and Eucalyptus oil.

Another embodiment comprises cyclosporin A, Antarox 25-R-2®, and Eucalyptus oil.

Another embodiment comprises cyclosporin A, Antarox 31-R-1®, and Eucalyptus oil.

Another embodiment comprises cyclosporin A, Antarox P-84®, and Eucalyptus oil.

Another embodiment comprises cyclosporin A, Antarox P-104/H®, and Eucalyptus oil.

Another embodiment comprises cyclosporin A, Arnox BP-Series®, and Eucalyptus oil.

Another embodiment comprises cyclosporin A, Chemonic 435®, and Eucalyptus oil.

Another embodiment comprises cyclosporin A, Chemonic D-25®, and Eucalyptus oil.

Another embodiment comprises cyclosporin A, Chemonic PL Series®, and Eucalyptus oil.

Another embodiment comprises cyclosporin A, Ethox L-121®, and Eucalyptus oil.

Another embodiment comprises cyclosporin A, Ethox L-122®, and Eucalyptus oil.

Another embodiment comprises cyclosporin A, Genapol PF-10®, and Eucalyptus oil.

Another embodiment comprises cyclosporin A, Genapol PF-20®, and Eucalyptus oil.

Another embodiment comprises cyclosporin A, Genapol PF-40A®, and Eucalyptus oil.

Another embodiment comprises cyclosporin A, Norfox 2-LF®, and Eucalyptus oil.

Another embodiment comprises cyclosporin A, Simulsol NW 342®, and Eucalyptus oil.

Another embodiment comprises cyclosporin A, T-Det BP-1®, and Eucalyptus oil.

Another embodiment comprises cyclosporin A, T-Det XD®, and Eucalyptus oil.

Another embodiment comprises cyclosporin A, T-Det XH®, and Eucalyptus oil.

Another embodiment comprises cyclosporin A, Triton CF-32®, and Eucalyptus oil.

Another embodiment comprises cyclosporin A, Witconol 171®, and Eucalyptus oil.

Another embodiment comprises cyclosporin A, Witconol 324®, and Eucalyptus oil.

Another embodiment comprises cyclosporin A, Witconol 324D®, and Eucalyptus oil.

Another embodiment comprises cyclosporin A, Witconol PD-2000®, and Eucalyptus oil.

Another embodiment comprises cyclosporin A, a Carboxylated Alcohol or Alkylphenol Ethoxylate, and Eucalyptus oil.

Another embodiment comprises cyclosporin A, Emcol CN-6®, and Eucalyptus oil.

Another embodiment comprises cyclosporin A, Ethcarb®, and Eucalyptus oil.

Another embodiment comprises cyclosporin A, Gemtex WNT-Conc®, and Eucalyptus oil.

Another embodiment comprises cyclosporin A, Incrodet TD7-C®, and Eucalyptus oil.

Another embodiment comprises cyclosporin A, Marlinat CM 105/80®, and Eucalyptus oil.

Another embodiment comprises cyclosporin A, Marlowet 1072®, and Eucalyptus oil.

Another embodiment comprises cyclosporin A, Marlowet 4530®, and Eucalyptus oil.

Another embodiment comprises cyclosporin A, Marlowet 4530 LF®, and Eucalyptus oil.

Another embodiment comprises cyclosporin A, Marlowet 4534®, and Eucalyptus oil.

Another embodiment comprises cyclosporin A, Marlowet 4538®, and Eucalyptus oil.

Another embodiment comprises cyclosporin A, Marlowet 4539®, and Eucalyptus oil.

Another embodiment comprises cyclosporin A, Marlowet 4539 LF®, and Eucalyptus oil.

Another embodiment comprises cyclosporin A, Marlowet 4541®, and Eucalyptus oil.

Another embodiment comprises cyclosporin A, Miranate LEC-80®, and Eucalyptus oil.

Another embodiment comprises cyclosporin A, Sandopan B®, and Eucalyptus oil.

Another embodiment comprises cyclosporin A, Sandopan B Modified®, and Eucalyptus oil.

Another embodiment comprises cyclosporin A, Sandopan LS-24 Gel®, and Eucalyptus oil.

Another embodiment comprises cyclosporin A, Surfine T-A®, and Eucalyptus oil.

Another embodiment comprises cyclosporin A, a Carboxylic Acid/Fatty Acid, and Eucalyptus oil.

Another embodiment comprises cyclosporin A, Colaterge RAM®, and Eucalyptus oil.

Another embodiment comprises cyclosporin A, Colatrope INC®, and Eucalyptus oil.

Another embodiment comprises cyclosporin A, Crodacid B®, and Eucalyptus oil.

Another embodiment comprises cyclosporin A, DeTrope CA-100®, and Eucalyptus oil.

Another embodiment comprises cyclosporin A, Latol MTO®, and Eucalyptus oil.

Another embodiment comprises cyclosporin A, Lumulse CC-33 K®, and Eucalyptus oil.

Another embodiment comprises cyclosporin A, Mulls 2218®, and Eucalyptus oil.

Another embodiment comprises cyclosporin A, OL-600®, and Eucalyptus oil.

Another embodiment comprises cyclosporin A, OL-800®, and Eucalyptus oil.

Another embodiment comprises cyclosporin A, R-910®, and Eucalyptus oil.

Another embodiment comprises cyclosporin A, S-210®, and Eucalyptus oil.

Another embodiment comprises cyclosporin A, Sandopan DTC Acid®, and Eucalyptus oil.

Another embodiment comprises cyclosporin A, Sandopan LS 24 N®, and Eucalyptus oil.

Another embodiment comprises cyclosporin A, Sandopan MA-18®, and Eucalyptus oil.

Another embodiment comprises cyclosporin A, an Ethoxylated Alcohol, and Eucalyptus oil.

Another embodiment comprises cyclosporin A, Adsee 799®, and Eucalyptus oil.

Another embodiment comprises cyclosporin A, Adsee 799®, and Eucalyptus oil.

Another embodiment comprises cyclosporin A, Alfonic 610-3.5®, and Eucalyptus oil.

Another embodiment comprises cyclosporin A, Alfonic 810-2®, and Eucalyptus oil.

Another embodiment comprises cyclosporin A, Alfonic 810-6®, and Eucalyptus oil.

Another embodiment comprises cyclosporin A, Alfonic 1012-3®, and Eucalyptus oil.

Another embodiment comprises cyclosporin A, Alfonic 1012-5®, and Eucalyptus oil.

Another embodiment comprises cyclosporin A, Alfonic 1216CO-1.5®, and Eucalyptus oil.

Another embodiment comprises cyclosporin A, Alfonic 1216CO-7®, and Eucalyptus oil.

Another embodiment comprises cyclosporin A, Alfonic 1412-3®, and Eucalyptus oil.

Another embodiment comprises cyclosporin A, Alfonic 1412-7®, and Eucalyptus oil.

Another embodiment comprises cyclosporin A, Arlasolve 200®, and Eucalyptus oil.

Another embodiment comprises cyclosporin A, Arlasolve 200 Liquid®, and Eucalyptus oil.

Another embodiment comprises cyclosporin A, Armix 180-C®, and Eucalyptus oil.

Another embodiment comprises cyclosporin A, Armix 183®, and Eucalyptus oil.

Another embodiment comprises cyclosporin A, Armul 2404®, and Eucalyptus oil.

Another embodiment comprises cyclosporin A, Armul 2404®, and Eucalyptus oil.

Another embodiment comprises cyclosporin A, Atlas EMJ-C®, and Eucalyptus oil.

Another embodiment comprises cyclosporin A, Atlas G-2109®, and Eucalyptus oil.

Another embodiment comprises cyclosporin A, Atlas G-3886®, and Eucalyptus oil.

Another embodiment comprises cyclosporin A, Atlas G-3890®, and Eucalyptus oil.

Another embodiment comprises cyclosporin A, Bio Soft E-200®, and Eucalyptus oil.

Another embodiment comprises cyclosporin A, Bio Soft E-300®, and Eucalyptus oil.

Another embodiment comprises cyclosporin A, Bio Soft E-400®, and Eucalyptus oil.

Another embodiment comprises cyclosporin A, Bio Soft EN 600®, and Eucalyptus oil.

Another embodiment comprises cyclosporin A, Bio Soft TD-400®, and Eucalyptus oil.

Another embodiment comprises cyclosporin A, Bio Soft TD-630®, and Eucalyptus oil.

Another embodiment comprises cyclosporin A, Brij 30®, and Eucalyptus oil.

Another embodiment comprises cyclosporin A, Brij 52®, and Eucalyptus oil.

Another embodiment comprises cyclosporin A, Brij 56®, and Eucalyptus oil.

Another embodiment comprises cyclosporin A, Brij 58®, and Eucalyptus oil.

Another embodiment comprises cyclosporin A, Brij 72®, and Eucalyptus oil.

Another embodiment comprises cyclosporin A, Brij 76®, and Eucalyptus oil.

Another embodiment comprises cyclosporin A, Brij 78®, and Eucalyptus oil.

Another embodiment comprises cyclosporin A, Brij 93®, and Eucalyptus oil.

Another embodiment comprises cyclosporin A, Brij 97®, and Eucalyptus oil.

Another embodiment comprises cyclosporin A, Brij 98®, and Eucalyptus oil.

Another embodiment comprises cyclosporin A, Brij 700®, and Eucalyptus oil.

Another embodiment comprises cyclosporin A, Brij 700 S®, and Eucalyptus oil.

Another embodiment comprises cyclosporin A, Brij 721®, and Eucalyptus oil.

Another embodiment comprises cyclosporin A, Brij 721 S®, and Eucalyptus oil.

Another embodiment comprises cyclosporin A, Burcoterge CDG®, and Eucalyptus oil.

Another embodiment comprises cyclosporin A, Canasol AT 600®, and Eucalyptus oil.

Another embodiment comprises cyclosporin A, Canasol AT 800®, and Eucalyptus oil.

Another embodiment comprises cyclosporin A, Canasol AT 1200®, and Eucalyptus oil.

Another embodiment comprises cyclosporin A, Canasol BJ 35®, and Eucalyptus oil.

Another embodiment comprises cyclosporin A, Canasol BJ 36®, and Eucalyptus oil.

Another embodiment comprises cyclosporin A, Canasol BJ 52®, and Eucalyptus oil.

Another embodiment comprises cyclosporin A, Canasol BJ 58®, and Eucalyptus oil.

Another embodiment comprises cyclosporin A, Canasol BJ 72®, and Eucalyptus oil.

Another embodiment comprises cyclosporin A, Canasol BJ 78®, and Eucalyptus oil.

Another embodiment comprises cyclosporin A, Canasol BJ 98®, and Eucalyptus oil.

Another embodiment comprises cyclosporin A, Canasol BJ 307®, and Eucalyptus oil.

Another embodiment comprises cyclosporin A, Cerfak 1400®, and Eucalyptus oil.

Another embodiment comprises cyclosporin A, Cetomacrogol 1000 BP®, and Eucalyptus oil.

Another embodiment comprises cyclosporin A, Chemonic C-2®, and Eucalyptus oil.

Another embodiment comprises cyclosporin A, Chemonic C-10®, and Eucalyptus oil.

Another embodiment comprises cyclosporin A, Chemonic C-20®, and Eucalyptus oil.

Another embodiment comprises cyclosporin A, Chemonic CT-12®, and Eucalyptus oil.

Another embodiment comprises cyclosporin A, Chemonic CT-20®, and Eucalyptus oil.

Another embodiment comprises cyclosporin A, Chemonic CT-30®, and Eucalyptus oil.

Another embodiment comprises cyclosporin A, Chemonic CT-55®, and Eucalyptus oil.

Another embodiment comprises cyclosporin A, Chemonic G-7®, and Eucalyptus oil.

Another embodiment comprises cyclosporin A, Chemonic G-26®, and Eucalyptus oil.

Another embodiment comprises cyclosporin A, Chemonic L-4®, and Eucalyptus oil.

Another embodiment comprises cyclosporin A, Chemonic L-7®, and Eucalyptus oil.

Another embodiment comprises cyclosporin A, Chemonic L-12®, and Eucalyptus oil.

Another embodiment comprises cyclosporin A, Chemonic L-23®, and Eucalyptus oil.

Another embodiment comprises cyclosporin A, Chemonic O-2®, and Eucalyptus oil.

Another embodiment comprises cyclosporin A, Chemonic O-5®, and Eucalyptus oil.

Another embodiment comprises cyclosporin A, Chemonic O-10®, and Eucalyptus oil.

Another embodiment comprises cyclosporin A, Chemonic O-20®, and Eucalyptus oil.

Another embodiment comprises cyclosporin A, Chemonic S-2®, and Eucalyptus oil.

Another embodiment comprises cyclosporin A, Chemonic S-10®, and Eucalyptus oil.

Another embodiment comprises cyclosporin A, Chemonic S-20®, and Eucalyptus oil.

Another embodiment comprises cyclosporin A, Colamulse FE®, and Eucalyptus oil.

Another embodiment comprises cyclosporin A, Cremophor A 20®, and Eucalyptus oil.

Another embodiment comprises cyclosporin A, Cremophor SA 2®, and Eucalyptus oil.

Another embodiment comprises cyclosporin A, Dehydol 100®, and Eucalyptus oil.

Another embodiment comprises cyclosporin A, Dehydol O-4®, and Eucalyptus oil.

Another embodiment comprises cyclosporin A, Delonic C-18®, and Eucalyptus oil.

Another embodiment comprises cyclosporin A, DeSonic 6T®, and Eucalyptus oil.

Another embodiment comprises cyclosporin A, DeSonic 9D®, and Eucalyptus oil.

Another embodiment comprises cyclosporin A, DeSonic 9T®, and Eucalyptus oil.

Another embodiment comprises cyclosporin A, DeSonic 12D®, and Eucalyptus oil.

Another embodiment comprises cyclosporin A, DeSonic 12T®, and Eucalyptus oil.

Another embodiment comprises cyclosporin A, DeSonic 15T®, and Eucalyptus oil.

Another embodiment comprises cyclosporin A, DeSonic TDA-9®, and Eucalyptus oil.

Another embodiment comprises cyclosporin A, DeThox GLG-7®, and Eucalyptus oil.

Another embodiment comprises cyclosporin A, DeThox GLG-26®, and Eucalyptus oil.

Another embodiment comprises cyclosporin A, DeThox LA-4®, and Eucalyptus oil.

Another embodiment comprises cyclosporin A, DeThox LA-23®, and Eucalyptus oil.

Another embodiment comprises cyclosporin A, DeThox SA-80®, and Eucalyptus oil.

Another embodiment comprises cyclosporin A, Disponil O5®, and Eucalyptus oil.

Another embodiment comprises cyclosporin A, Eccoterge EO-41B®, and Eucalyptus oil.

Another embodiment comprises cyclosporin A, Empilan KA2.5/90®, and Eucalyptus oil.

Another embodiment comprises cyclosporin A, Empilan KA5/90®, and Eucalyptus oil.

Another embodiment comprises cyclosporin A, Empilan KM-20®, and Eucalyptus oil.

Another embodiment comprises cyclosporin A, Empilan KM-50®, and Eucalyptus oil.

Another embodiment comprises cyclosporin A, Empilan L-23®, and Eucalyptus oil.

Another embodiment comprises cyclosporin A, Ethylan 25-3®, and Eucalyptus oil.

Another embodiment comprises cyclosporin A, Ethylan 1204®, and Eucalyptus oil.

Another embodiment comprises cyclosporin A, Ethylan DA-4®, and Eucalyptus oil.

Another embodiment comprises cyclosporin A, Ethylan LA-230®, and Eucalyptus oil.

Another embodiment comprises cyclosporin A, Ethylan SN®, and Eucalyptus oil.

Another embodiment comprises cyclosporin A, Ethylan TD-60®, and Eucalyptus oil.

Another embodiment comprises cyclosporin A, Ethylan TD-100®, and Eucalyptus oil.

Another embodiment comprises cyclosporin A, Ethylan TD-1407®, and Eucalyptus oil.

Another embodiment comprises cyclosporin A, Eumulgin B1®, and Eucalyptus oil.

Another embodiment comprises cyclosporin A, Eumulgin B2®, and Eucalyptus oil.

Another embodiment comprises cyclosporin A, Eumulgin B3®, and Eucalyptus oil.

Another embodiment comprises cyclosporin A, Eumulgin O-10®, and Eucalyptus oil.

Another embodiment comprises cyclosporin A, Flo Mo 80/20®, and Eucalyptus oil.

Another embodiment comprises cyclosporin A, Flo Mo Low Foam®, and Eucalyptus oil.

Another embodiment comprises cyclosporin A, Forlan C-24®, and Eucalyptus oil.

Another embodiment comprises cyclosporin A, Genapol 1454®, and Eucalyptus oil.

Another embodiment comprises cyclosporin A, Genapol BA-020®, and Eucalyptus oil.

Another embodiment comprises cyclosporin A, Genapol BA-040®, and Eucalyptus oil.

Another embodiment comprises cyclosporin A, Genapol C-100®, and Eucalyptus oil.

Another embodiment comprises cyclosporin A, Genapol DA 060®, and Eucalyptus oil.

Another embodiment comprises cyclosporin A, Genapol HS 020®, and Eucalyptus oil.

Another embodiment comprises cyclosporin A, Genapol HS 200®, and Eucalyptus oil.

Another embodiment comprises cyclosporin A, Genapol ID-040®, and Eucalyptus oil.

Another embodiment comprises cyclosporin A, Genapol ID-060®, and Eucalyptus oil.

Another embodiment comprises cyclosporin A, Genapol ID-090®, and Eucalyptus oil.

Another embodiment comprises cyclosporin A, Genapol LA 010®, and Eucalyptus oil.

Another embodiment comprises cyclosporin A, Genapol LA 020®, and Eucalyptus oil.

Another embodiment comprises cyclosporin A, Genapol LA 030®, and Eucalyptus oil.

Another embodiment comprises cyclosporin A, Genapol LA 040®, and Eucalyptus oil.

Another embodiment comprises cyclosporin A, Genapol LA 050®, and Eucalyptus oil.

Another embodiment comprises cyclosporin A, Genapol LA 060®, and Eucalyptus oil.

Another embodiment comprises cyclosporin A, Genapol LA 070®, and Eucalyptus oil.

Another embodiment comprises cyclosporin A, Genapol LA 070S®, and Eucalyptus oil.

Another embodiment comprises cyclosporin A, Genapol LA 230®, and Eucalyptus oil.

Another embodiment comprises cyclosporin A, Genapol O 020®, and Eucalyptus oil.

Another embodiment comprises cyclosporin A, Genapol O 050®, and Eucalyptus oil.

Another embodiment comprises cyclosporin A, Genapol O 100®, and Eucalyptus oil.

Another embodiment comprises cyclosporin A, Genapol O 200®, and Eucalyptus oil.

Another embodiment comprises cyclosporin A, Genapol SA 030®, and Eucalyptus oil.

Another embodiment comprises cyclosporin A, Genapol SA 120®, and Eucalyptus oil.

Another embodiment comprises cyclosporin A, Genapol T-020®, and Eucalyptus oil.

Another embodiment comprises cyclosporin A, Genapol UD-030®, and Eucalyptus oil.

Another embodiment comprises cyclosporin A, Genapol UD-050®, and Eucalyptus oil.

Another embodiment comprises cyclosporin A, Genapol UD-070®, and Eucalyptus oil.

Another embodiment comprises cyclosporin A, Genapol UD-079®, and Eucalyptus oil.

Another embodiment comprises cyclosporin A, Genapol UD-080®, and Eucalyptus oil.

Another embodiment comprises cyclosporin A, Genapol UD-110®, and Eucalyptus oil.

Another embodiment comprises cyclosporin A, Genapol X 030®, and Eucalyptus oil.

Another embodiment comprises cyclosporin A, Genapol X 050®, and Eucalyptus oil.

Another embodiment comprises cyclosporin A, Genapol X 060®, and Eucalyptus oil.

Another embodiment comprises cyclosporin A, Genapol X 070®, and Eucalyptus oil.

Another embodiment comprises cyclosporin A, Genapol X 080®, and Eucalyptus oil.

Another embodiment comprises cyclosporin A, Genapol X 100®, and Eucalyptus oil.

Another embodiment comprises cyclosporin A, Genapol X159®, and Eucalyptus oil.

Another embodiment comprises cyclosporin A, Generol 122 E5®, and Eucalyptus oil.

Another embodiment comprises cyclosporin A, Generol 122 E25®, and Eucalyptus oil.

Another embodiment comprises cyclosporin A, Hostacerin T-3®, and Eucalyptus oil.

Another embodiment comprises cyclosporin A, Iconol LF 110®, and Eucalyptus oil.

Another embodiment comprises cyclosporin A, Incropol CS-20®, and Eucalyptus oil.

Another embodiment comprises cyclosporin A, Lexemul CS-20®, and Eucalyptus oil.

Another embodiment comprises cyclosporin A, Liponic EG-1®, and Eucalyptus oil.

Another embodiment comprises cyclosporin A, Lipowax D®, and Eucalyptus oil.

Another embodiment comprises cyclosporin A, Lipowax G®, and Eucalyptus oil.

Another embodiment comprises cyclosporin A, Lipowax NI®, and Eucalyptus oil.

Another embodiment comprises cyclosporin A, Lipowax P®, and Eucalyptus oil.

Another embodiment comprises cyclosporin A, Lipowax P-31®, and Eucalyptus oil.

Another embodiment comprises cyclosporin A, Lipowax PR®, and Eucalyptus oil.

Another embodiment comprises cyclosporin A, Lumulse CS-20®, and Eucalyptus oil.

Another embodiment comprises cyclosporin A, Macol CSA-20®, and Eucalyptus oil.

Another embodiment comprises cyclosporin A, Marlox B 24/50®, and Eucalyptus oil.

Another embodiment comprises cyclosporin A, Mazawet 77®, and Eucalyptus oil.

Another embodiment comprises cyclosporin A, Nortox 1713®, and Eucalyptus oil.

Another embodiment comprises cyclosporin A, Norfox 2579®, and Eucalyptus oil.

Another embodiment comprises cyclosporin A, Norfox Lo Foam®, and Eucalyptus oil.

Another embodiment comprises cyclosporin A, Promulgen D®, and Eucalyptus oil.

Another embodiment comprises cyclosporin A, Promulgen G®, and Eucalyptus oil.

Another embodiment comprises cyclosporin A, Renex 30®, and Eucalyptus oil.

Another embodiment comprises cyclosporin A, Renex 36®, and Eucalyptus oil.

Another embodiment comprises cyclosporin A, Rhodasurf A 24®, and Eucalyptus oil.

Another embodiment comprises cyclosporin A, Rhodasurf AAE-10®, and Eucalyptus oil.

Another embodiment comprises cyclosporin A, Rhodasurf BEH-25®, and Eucalyptus oil.

Another embodiment comprises cyclosporin A, Rhodasurf BEH-40®, and Eucalyptus oil.

Another embodiment comprises cyclosporin A, Rhodasurf DA 530®, and Eucalyptus oil.

Another embodiment comprises cyclosporin A, Rhodasunf DA 630®, and Eucalyptus oil.

Another embodiment comprises cyclosporin A, Rhodasud DA 639®, and Eucalyptus oil.

Another embodiment comprises cyclosporin A, Rhodasurf LAN-23®, and Eucalyptus oil.

Another embodiment comprises cyclosporin A, Rhodasurf ON-870®, and Eucalyptus oil.

Another embodiment comprises cyclosporin A, Rhodasurf ON-877®, and Eucalyptus oil.

Another embodiment comprises cyclosporin A, Rhodasurf TB-970 FLK®, and Eucalyptus oil.

Another embodiment comprises cyclosporin A, Ritacet-20®, and Eucalyptus oil.

Another embodiment comprises cyclosporin A, Ritachol 1000®, and Eucalyptus oil.

Another embodiment comprises cyclosporin A, Ritachol 2000®, and Eucalyptus oil.

Another embodiment comprises cyclosporin A, Ritachol 5000®, and Eucalyptus oil.

Another embodiment comprises cyclosporin A, Ritox 35®, and Eucalyptus oil.

Another embodiment comprises cyclosporin A, Surfonic DA-4®, and Eucalyptus oil.

Another embodiment comprises cyclosporin A, Surfonic DA-6®, and Eucalyptus oil.

Another embodiment comprises cyclosporin A, Surfonic L46-7®, and Eucalyptus oil.

Another embodiment comprises cyclosporin A, Surfonic POA®, and Eucalyptus oil.

Another embodiment comprises cyclosporin A, Synthrapol KB®, and Eucalyptus oil.

Another embodiment comprises cyclosporin A, Teginacid®, and Eucalyptus oil.

Another embodiment comprises cyclosporin A, Teginacid C®, and Eucalyptus oil.

Another embodiment comprises cyclosporin A, Tegotens EC 11®, and Eucalyptus oil.

Another embodiment comprises cyclosporin A, Tinegal NA®, and Eucalyptus oil.

Another embodiment comprises cyclosporin A, Tomadol 400®, and Eucalyptus oil.

Another embodiment comprises cyclosporin A, Tomadol 600®, and Eucalyptus oil.

Another embodiment comprises cyclosporin A, Tomadol 900®, and Eucalyptus oil.

Another embodiment comprises cyclosporin A, Uniperol O®, and Eucalyptus oil.

Another embodiment comprises cyclosporin A, Witconol SN Series®, and Eucalyptus oil.

Another embodiment comprises cyclosporin A, an Ethoxylated Alkylphenol, and Eucalyptus oil.

Another embodiment comprises cyclosporin A, Antarox LF-222®, and Eucalyptus oil.

Another embodiment comprises cyclosporin A, Atlox 775®, and Eucalyptus oil.

Another embodiment comprises cyclosporin A, Caloxylate N-9®, and Eucalyptus oil.

Another embodiment comprises cyclosporin A, Canasol NF-1000®, and Eucalyptus oil.

Another embodiment comprises cyclosporin A, Canasol NF-3000®, and Eucalyptus oil.

Another embodiment comprises cyclosporin A, Canasol NF-3070®, and Eucalyptus oil.

Another embodiment comprises cyclosporin A, Canasol OF 1670®, and Eucalyptus oil.

Another embodiment comprises cyclosporin A, Canasol OF 2570®, and Eucalyptus oil.

Another embodiment comprises cyclosporin A, Canasol OF 4070®, and Eucalyptus oil.

Another embodiment comprises cyclosporin A, Chemax DNP-8®, and Eucalyptus oil.

Another embodiment comprises cyclosporin A, Chemax DNP-18®, and Eucalyptus oil.

Another embodiment comprises cyclosporin A, Chemax DNP-150/50®, and Eucalyptus oil.

Another embodiment comprises cyclosporin A, DeSonic 1.5N®, and Eucalyptus oil.

Another embodiment comprises. cyclosporin A, DeSonic 4N®, and Eucalyptus oil.

Another embodiment comprises cyclosporin A, DeSonic 5N®, and Eucalyptus oil.

Another embodiment comprises cyclosporin A, DeSonic 6D®, and Eucalyptus oil.

Another embodiment comprises cyclosporin A, DeSonic 6N®, and Eucalyptus oil.

Another embodiment comprises cyclosporin A, DeSonic 7N®, and Eucalyptus oil.

Another embodiment comprises cyclosporin A, DeSonic 9N®, and Eucalyptus oil.

Another embodiment comprises cyclosporin A, DeSonic 10D®, and Eucalyptus oil.

Another embodiment comprises cyclosporin A, DeSonic 11N®, and Eucalyptus oil.

Another embodiment comprises cyclosporin A, DeSonic 12N®, and Eucalyptus oil.

Another embodiment comprises cyclosporin A, DeSonic 15N®, and Eucalyptus oil.

Another embodiment comprises cyclosporin A, DeSonic 20N®, and Eucalyptus oil.

Another embodiment comprises cyclosporin A, Eccoscour RC®, and Eucalyptus oil.

Another embodiment comprises cyclosporin A, Eccoterge EO-100®, and Eucalyptus oil.

Another embodiment comprises cyclosporin A, Emulsifier 632/90%®, and Eucalyptus oil.

Another embodiment comprises cyclosporin A, Geronol AG-821®, and Eucalyptus oil.

Another embodiment comprises cyclosporin A, Gradonic N-95®, and Eucalyptus oil.

Another embodiment comprises cyclosporin A, Hetoxide NP-4®, and Eucalyptus oil.

Another embodiment comprises cyclosporin A, Hetoxide NP-30®, and Eucalyptus oil.

Another embodiment comprises cyclosporin A, Hostapal N-100®, and Eucalyptus oil.

Another embodiment comprises cyclosporin A, Hostapal N-110®, and Eucalyptus oil.

Another embodiment comprises cyclosporin A, Igepal CTA-639W®, and Eucalyptus oil.

Another embodiment comprises cyclosporin A, Igepal DAP-9®, and Eucalyptus oil.

Another embodiment comprises cyclosporin A, Igepal OD-410®, and Eucalyptus oil.

Another embodiment comprises cyclosporin A, Igepal SS-837®, and Eucalyptus oil.

Another embodiment comprises cyclosporin A, Lipocol NP-9 USP®, and Eucalyptus oil.

Another embodiment comprises cyclosporin A, Macol DNP-10®, and Eucalyptus oil.

Another embodiment comprises cyclosporin A, Marlophen NP 5®, and Eucalyptus oil.

Another embodiment comprises cyclosporin A, Marlophen P 1®, and Eucalyptus oil.

Another embodiment comprises cyclosporin A, Surfonic NB®, and Eucalyptus oil.

Another embodiment comprises cyclosporin A, Surfonic OPB-307®, and Eucalyptus oil.

Another embodiment comprises cyclosporin A, Surfonic OPB-407®, and Eucalyptus oil.

Another embodiment comprises cyclosporin A, Syn Fac 334®, and Eucalyptus oil.

Another embodiment comprises cyclosporin A, Syn Fac 8216®, and Eucalyptus oil.

Another embodiment comprises cyclosporin A, Triton N-57®, and Eucalyptus oil.

Another embodiment comprises cyclosporin A, Trycol 6956®, and Eucalyptus oil.

Another embodiment comprises cyclosporin A, Trycol 6961®, and Eucalyptus oil.

Another embodiment comprises cyclosporin A, Trycol 6964®, and Eucalyptus oil.

Another embodiment comprises cyclosporin A, Trycol 6969®, and Eucalyptus oil.

Another embodiment comprises cyclosporin A, Trycol 6974®, and Eucalyptus oil.

Another embodiment comprises cyclosporin A, Witbreak DRB-127®, and Eucalyptus oil.

Another embodiment comprises cyclosporin A, Witbreak DRB-127®, and Eucalyptus oil.

Another embodiment comprises cyclosporin A, Witconol NP Series®, and Eucalyptus oil.

Another embodiment comprises cyclosporin A, an Ethoxylated Aryl Phenol, and Eucalyptus oil.

Another embodiment comprises cyclosporin A, Soprophor BSU®, and Eucalyptus oil.

Another embodiment comprises cyclosporin A, Soprophor CY/8®, and Eucalyptus oil.

Another embodiment comprises cyclosporin A, Soprophor S/25®, and Eucalyptus oil.

Another embodiment comprises cyclosporin A, Witconol NIO®, and Eucalyptus oil.

Another embodiment comprises cyclosporin A, Witconol NIW®, and Eucalyptus oil.

Another embodiment comprises cyclosporin A, Witconol S-100®, and Eucalyptus oil.

Another embodiment comprises cyclosporin A, an Ethoxylated Fatty Acid, and Eucalyptus oil.

Another embodiment comprises cyclosporin A, Aldo PGHMS®, and Eucalyptus oil.

Another embodiment comprises cyclosporin A, Alkamuls TO-15/HR®, and Eucalyptus oil.

Another embodiment comprises cyclosporin A, Armotan AL-69-66®, and Eucalyptus oil.

Another embodiment comprises cyclosporin A, Cerasynt 840®, and Eucalyptus oil.

Another embodiment comprises cyclosporin A, Cerasynt 945®, and Eucalyptus oil.

Another embodiment comprises cyclosporin A, Crystal Inhibitor No. 5®, and Eucalyptus oil.

Another embodiment comprises cyclosporin A, DeThox Acid L-9®, and Eucalyptus oil.

Another embodiment comprises cyclosporin A, DeThox Acid S-8®, and Eucalyptus oil.

Another embodiment comprises cyclosporin A, Ethofat 242/25®, and Eucalyptus oil.

Another embodiment comprises cyclosporin A, Hydropalat 65®, and Eucalyptus oil.

Another embodiment comprises cyclosporin A, Lipo EGMS®, and Eucalyptus oil.

Another embodiment comprises cyclosporin A, Lipopeg 2 DL®, and Eucalyptus oil.

Another embodiment comprises cyclosporin A, Lipopeg 4 DL®, and Eucalyptus oil.

Another embodiment comprises cyclosporin A, Lipopeg 4-L®, and Eucalyptus oil.

Another embodiment comprises cyclosporin A, Lipopeg 39-S®, and Eucalyptus oil.

Another embodiment comprises cyclosporin A, Lipopeg 4-S®, and Eucalyptus oil.

Another embodiment comprises cyclosporin A, Lipopeg 10-S®, and Eucalyptus oil.

Another embodiment comprises cyclosporin A, Lipopeg 100-S®, and Eucalyptus oil.

Another embodiment comprises cyclosporin A, Lipopeg 6000 DS®, and Eucalyptus oil.

Another embodiment comprises cyclosporin A, Lumulse 40-L®, and Eucalyptus oil.

Another embodiment comprises cyclosporin A, Lumulse 40-S®, and Eucalyptus oil.

Another embodiment comprises cyclosporin A, Lumulse 42-L®, and Eucalyptus oil.

Another embodiment comprises cyclosporin A, Lumulse 42-S®, and Eucalyptus oil.

Another embodiment comprises cyclosporin A, Lumulse 100-S®, and Eucalyptus oil.

Another embodiment comprises cyclosporin A, Lumulse 602-S®, and Eucalyptus oil.

Another embodiment comprises cyclosporin A, Magrabar PGE-20-0®, and Eucalyptus oil.

Another embodiment comprises cyclosporin A, Magrabar PGE-20L®, and Eucalyptus oil.

Another embodiment comprises cyclosporin A, Magrabar PGE-20T®, and Eucalyptus oil.

Another embodiment comprises cyclosporin A, Magrabar PGE-22-0®, and Eucalyptus oil.

Another embodiment comprises cyclosporin A, Magrabar PGE-22L®, and Eucalyptus oil.

Another embodiment comprises cyclosporin A, Magrabar PGE-22T®, and Eucalyptus oil.

Another embodiment comprises cyclosporin A, Magrabar PGE-40-0®, and Eucalyptus oil.

Another embodiment comprises cyclosporin A, Magrabar PGE-40L®, and Eucalyptus oil.

Another embodiment comprises cyclosporin A, Magrabar PGE-40T®, and Eucalyptus oil.

Another embodiment comprises cyclosporin A, Magrabar PGE-42-0®, and Eucalyptus oil.

Another embodiment comprises cyclosporin A, Magrabar PGE-42L®, and Eucalyptus oil.

Another embodiment comprises cyclosporin A, Magrabar PGE-42T®, and Eucalyptus oil.

Another embodiment comprises cyclosporin A, Magrabar PGE-60-0®, and Eucalyptus oil.

Another embodiment comprises cyclosporin A, Magrabar PGE-60L®, and Eucalyptus oil.

Another embodiment comprises cyclosporin A, Magrabar PGE-60T®, and Eucalyptus oil.

Another embodiment comprises cyclosporin A, Magrabar PGE-62-0®, and Eucalyptus oil.

Another embodiment comprises cyclosporin A, Magrabar PGE-62L®, and Eucalyptus oil.

Another embodiment comprises cyclosporin A, Magrabar PGE-62T®, and Eucalyptus oil.

Another embodiment comprises cyclosporin A, Mapeg S-40K®, and Eucalyptus oil.

Another embodiment comprises cyclosporin A, Marlowet OTS®, and Eucalyptus oil.

Another embodiment comprises cyclosporin A, Naturechem PGR®, and Eucalyptus oil.

Another embodiment comprises cyclosporin A, PG No. 4®, and Eucalyptus oil.

Another embodiment comprises cyclosporin A, Renex 20®, and Eucalyptus oil.

Another embodiment comprises cyclosporin A, Ritox 52®, and Eucalyptus oil.

Another embodiment comprises cyclosporin A, Ritox 53®, and Eucalyptus oil.

Another embodiment comprises cyclosporin A, Ritox 59®, and Eucalyptus oil.

Another embodiment comprises cyclosporin A, Surfax 8916/A®, and Eucalyptus oil.

Another embodiment comprises cyclosporin A, Tego Acid S 40 P®, and Eucalyptus oil.

Another embodiment comprises cyclosporin A, Tego Acid S 100 P®, and Eucalyptus oil.

Another embodiment comprises cyclosporin A, Tween 20®, and Eucalyptus oil.

Another embodiment comprises cyclosporin A, Volpo 131®, and Eucalyptus oil.

Another embodiment comprises cyclosporin A, an Ethoxylated Fatty Ester or Oil, and Eucalyptus oil.

Another embodiment comprises cyclosporin A, Acconon 6-C10®, and Eucalyptus oil.

Another embodiment comprises cyclosporin A, Acconon CC-6®, and Eucalyptus oil.

Another embodiment comprises cyclosporin A, Acconon CO-7®, and Eucalyptus oil.

Another embodiment comprises cyclosporin A, Aldosperse 40/60 FG®, and Eucalyptus oil.

Another embodiment comprises cyclosporin A, Aldosperse ML-23®, and Eucalyptus oil.

Another embodiment comprises cyclosporin A, Aldosperse MS-20 FG®, and Eucalyptus oil.

Another embodiment comprises cyclosporin A, Alkamuls EL-620®, and Eucalyptus oil.

Another embodiment comprises cyclosporin A, Alkamuls EL-719®, and Eucalyptus oil.

Another embodiment comprises cyclosporin A, Alkamuls EL-985®, and Eucalyptus oil.

Another embodiment comprises cyclosporin A, Arlatone G®, and Eucalyptus oil.

Another embodiment comprises cyclosporin A, Arlatone T®, and Eucalyptus oil.

Another embodiment comprises cyclosporin A, Atlas G-1045A®, and Eucalyptus oil.

Another embodiment comprises cyclosporin A, Atlas G-1086®, and Eucalyptus oil.

Another embodiment comprises cyclosporin A, Atlas G-1087®, and Eucalyptus oil.

Another embodiment comprises cyclosporin A, Atlas G-1089®, and Eucalyptus oil.

Another embodiment comprises cyclosporin A, Atlas G-1096®, and Eucalyptus oil.

Another embodiment comprises cyclosporin A, Atlas G-1292®, and Eucalyptus oil.

Another embodiment comprises cyclosporin A, Atlas G-1293®, and Eucalyptus oil.

Another embodiment comprises cyclosporin A, Atlas G-1300®, and Eucalyptus oil.

Another embodiment comprises cyclosporin A, Atlas G-7076®, and Eucalyptus oil.

Another embodiment comprises cyclosporin A, Capmul EMG®, and Eucalyptus oil.

Another embodiment comprises cyclosporin A, Chemonic CO-40®, and Eucalyptus oil.

Another embodiment comprises cyclosporin A, Chemonic LI-3®, and Eucalyptus oil.

Another embodiment comprises cyclosporin A, Chemonic LI-7®, and Eucalyptus oil.

Another embodiment comprises cyclosporin A, Cirrasol GM®, and Eucalyptus oil.

Another embodiment comprises cyclosporin A, Cremophor CO 40®, and Eucalyptus oil.

Another embodiment comprises cyclosporin A, Cremophor CO 410®, and Eucalyptus oil.

Another embodiment comprises cyclosporin A, Cremophor EL®, and Eucalyptus oil.

Another embodiment comprises cyclosporin A, Cremophor GC7®, and Eucalyptus oil.

Another embodiment comprises cyclosporin A, Cremophor RH-40®, and Eucalyptus oil.

Another embodiment comprises cyclosporin A, Crovol A-40®, and Eucalyptus oil.

Another embodiment comprises cyclosporin A, Crovol A-70®, and Eucalyptus oil.

Another embodiment comprises cyclosporin A, Crovol M-70®, and Eucalyptus oil.

Another embodiment comprises cyclosporin A, Crovol PK-70®, and Eucalyptus oil.

Another embodiment comprises cyclosporin A, Cutina E-24®, and Eucalyptus oil.

Another embodiment comprises cyclosporin A, Dacospin 12-R®, and Eucalyptus oil.

Another embodiment comprises cyclosporin A, Dehymuls HRE-7®, and Eucalyptus oil.

Another embodiment comprises cyclosporin A, DeSonic 30C®, and Eucalyptus oil.

Another embodiment comprises cyclosporin A, DeSonic 36C®, and Eucalyptus oil.

Another embodiment comprises cyclosporin A, DeSonic 40C, and Eucalyptus oil.

Another embodiment comprises cyclosporin A, Durfax 60®, and Eucalyptus oil.

Another embodiment comprises cyclosporin A, Durfax 65®, and Eucalyptus oil.

Another embodiment comprises cyclosporin A, Durfax 80®, and Eucalyptus oil.

Another embodiment comprises cyclosporin A, Durfax EOM®, and Eucalyptus oil.

Another embodiment comprises cyclosporin A, Eccoterge NF-2®, and Eucalyptus oil.

Another embodiment comprises cyclosporin A, Emulpon CO-360®, and Eucalyptus oil.

Another embodiment comprises cyclosporin A, Emulpon CO-550®, and Eucalyptus oil.

Another embodiment comprises cyclosporin A, Emulsogen EL®, and Eucalyptus oil.

Another embodiment comprises cyclosporin A, Emulsogen HCO 040®, and Eucalyptus oil.

Another embodiment comprises cyclosporin A, Emulsogen HCO 060®, and Eucalyptus oil.

Another embodiment comprises cyclosporin A, Emulsynt 1055®, and Eucalyptus oil.

Another embodiment comprises cyclosporin A, Ethox 3095®, and Eucalyptus oil.

Another embodiment comprises cyclosporin A, Eumulgin RO-40®, and Eucalyptus oil.

Another embodiment comprises cyclosporin A, Genapol G-260®, and Eucalyptus oil.

Another embodiment comprises cyclosporin A, Glycosperse L-20®, and Eucalyptus oil.

Another embodiment comprises cyclosporin A, Glycosperse O-5®, and Eucalyptus oil.

Another embodiment comprises cyclosporin A, Glycosperse O-20®, and Eucalyptus oil.

Another embodiment comprises cyclosporin A, Glycosperse O-20 FG®, and Eucalyptus oil.

Another embodiment comprises cyclosporin A, Glycosperse S-20®, and Eucalyptus oil.

Another embodiment comprises cyclosporin A, Glycosperse S-20 FG®, and Eucalyptus oil.

Another embodiment comprises cyclosporin A, Glycosperse TS-20®, and Eucalyptus oil.

Another embodiment comprises cyclosporin A, Glycosperse TS-20 FG®, and Eucalyptus oil.

Another embodiment comprises cyclosporin A, Hetan SL®, and Eucalyptus oil.

Another embodiment comprises cyclosporin A, Hetan SO®, and Eucalyptus oil.

Another embodiment comprises cyclosporin A, Hetan SS®, and Eucalyptus oil.

Another embodiment comprises cyclosporin A, Hetoxide C-2®, and Eucalyptus oil.

Another embodiment comprises cyclosporin A, Hetoxide C-9®, and Eucalyptus oil.

Another embodiment comprises cyclosporin A, Hetoxide C-15®, and Eucalyptus oil.

Another embodiment comprises cyclosporin A, Hetoxide C-25®, and Eucalyptus oil.

Another embodiment comprises cyclosporin A, Hetoxide C-40®, and Eucalyptus oil.

Another embodiment comprises cyclosporin A, Hetoxide C-200®, and Eucalyptus oil.

Another embodiment comprises cyclosporin A, Hetoxide C-200-50%®, and Eucalyptus oil.

Another embodiment comprises cyclosporin A, Hetoxide GC-30®, and Eucalyptus oil.

Another embodiment comprises cyclosporin A, Hetoxide HC-60®, and Eucalyptus oil.

Another embodiment comprises cyclosporin A, Ice No. 2®, and Eucalyptus oil.

Another embodiment comprises cyclosporin A, Incrocas 30/40®, and Eucalyptus oil.

Another embodiment comprises cyclosporin A, Lexol EC®, and Eucalyptus oil.

Another embodiment comprises cyclosporin A, Lexol EO®, and Eucalyptus oil.

Another embodiment comprises cyclosporin A, Lipocol HCO-40®, and Eucalyptus oil.

Another embodiment comprises cyclosporin A, Lipocol HCO-60®, and Eucalyptus oil.

Another embodiment comprises cyclosporin A, Lipocol O-3 Special®, and Eucalyptus oil.

Another embodiment comprises cyclosporin A, Lipopeg 2-L®, and Eucalyptus oil.

Another embodiment comprises cyclosporin A, Lipopeg 4-DO®, and Eucalyptus oil.

Another embodiment comprises cyclosporin A, Lipopeg 4-DS®, and Eucalyptus oil.

Another embodiment comprises cyclosporin A, Lipovol GTB®, and Eucalyptus oil.

Another embodiment comprises cyclosporin A, Lonzest SML-20®, and Eucalyptus oil.

Another embodiment comprises cyclosporin A, Lonzest SMO-20®, and Eucalyptus oil.

Another embodiment comprises cyclosporin A, Lonzest SMS-20®, and Eucalyptus oil.

Another embodiment comprises cyclosporin A, Lonzest STO-20®, and Eucalyptus oil.

Another embodiment comprises cyclosporin A, Lonzest STS-20®, and Eucalyptus oil.

Another embodiment comprises cyclosporin A, Lumulse GR-40®, and Eucalyptus oil.

Another embodiment comprises cyclosporin A, Lumulse GRH-40®, and Eucalyptus oil.

Another embodiment comprises cyclosporin A, Lumulse POE (7) GML®, and Eucalyptus oil.

Another embodiment comprises cyclosporin A, Lumulse POE (12) Glyc®, and Eucalyptus oil.

Another embodiment comprises cyclosporin A, Lumulse POE (40) MS KP®, and Eucalyptus oil.

Another embodiment comprises cyclosporin A, Marlowet 4750®, and Eucalyptus oil.

Another embodiment comprises cyclosporin A, Marlowet LVS®, and Eucalyptus oil.

Another embodiment comprises cyclosporin A, Marlowet R 11®, and Eucalyptus oil.

Another embodiment comprises cyclosporin A, Marlowet R 40®, and Eucalyptus oil.

Another embodiment comprises cyclosporin A, Mazol 80 MGK®, and Eucalyptus oil.

Another embodiment comprises cyclosporin A, Nonionic Emulsifier T-9®, and Eucalyptus oil.

Another embodiment comprises cyclosporin A, Oronal LCG®, and Eucalyptus oil.

Another embodiment comprises cyclosporin A, Polyderm PPI-CO-200®, and Eucalyptus oil.

Another embodiment comprises cyclosporin A, Polyderm PPI-CO-40®, and Eucalyptus oil.

Another embodiment comprises cyclosporin A, Rewoderm LI 520-70®, and Eucalyptus oil.

Another embodiment comprises cyclosporin A, Ritapeg 150 DS®, and Eucalyptus oil.

Another embodiment comprises cyclosporin A, Softigen 767®, and Eucalyptus oil.

Another embodiment comprises cyclosporin A, Surfactol 318®, and Eucalyptus oil.

Another embodiment comprises cyclosporin A, Surfactol 365®, and Eucalyptus oil.

Another embodiment comprises cyclosporin A, Syn Lube 107®, and Eucalyptus oil.

Another embodiment comprises cyclosporin A, Syn Lube 728®, and Eucalyptus oil.

Another embodiment comprises cyclosporin A, Syn Lube 1632H®, and Eucalyptus oil.

Another embodiment comprises cyclosporin A, Syn Lube 6277-A®, and Eucalyptus oil.

Another embodiment comprises cyclosporin A, T-Det C-20®, and Eucalyptus oil.

Another embodiment comprises cyclosporin A, T-Det C-40®, and Eucalyptus oil.

Another embodiment comprises cyclosporin A, Tally 100 Plus®, and Eucalyptus oil.

Another embodiment comprises cyclosporin A, Uniperol EL®, and Eucalyptus oil.

Another embodiment comprises cyclosporin A, a Fatty Ester, and Eucalyptus oil.

Another embodiment comprises cyclosporin A, Actralube-Syn 147®, and Eucalyptus oil.

Another embodiment comprises cyclosporin A, Atlas G-1556®, and Eucalyptus oil.

Another embodiment comprises cyclosporin A, Atlas G-1564®, and Eucalyptus oil.

Another embodiment comprises cyclosporin A, Atlasol Base Oil S®, and Eucalyptus oil.

Another embodiment comprises cyclosporin A, Base ML®, and Eucalyptus oil.

Another embodiment comprises cyclosporin A, Base MT®, and Eucalyptus oil.

Another embodiment comprises cyclosporin A, Cerasynt 303®, and Eucalyptus oil.

Another embodiment comprises cyclosporin A, Dermol 1012®, and Eucalyptus oil.

Another embodiment comprises cyclosporin A, Kemester 4000®, and Eucalyptus oil.

Another embodiment comprises cyclosporin A, Lactipol S®, and Eucalyptus oil.

Another embodiment comprises cyclosporin A, Magrabar PGO®, and Eucalyptus oil.

Another embodiment comprises cyclosporin A, Mayco Base BFO®, and Eucalyptus oil.

Another embodiment comprises cyclosporin A, Methyl Linoleate®, and Eucalyptus oil.

Another embodiment comprises cyclosporin A, Pationic 122A®, and Eucalyptus oil.

Another embodiment comprises cyclosporin A, Pationic 138C®, and Eucalyptus oil.

Another embodiment comprises cyclosporin A, Pationic CSL®, and Eucalyptus oil.

Another embodiment comprises cyclosporin A, Pationic ISL®, and Eucalyptus oil.

Another embodiment comprises cyclosporin A, Pationic SBL®, and Eucalyptus oil.

Another embodiment comprises cyclosporin A, Pationic SSL®, and Eucalyptus oil.

Another embodiment comprises cyclosporin A, Ritasol®, and Eucalyptus oil.

Another embodiment comprises cyclosporin A, Tego Alkanol CS 20®, and Eucalyptus oil.

Another embodiment comprises cyclosporin A, Tego Alkanol L23 P®, and Eucalyptus oil.

Another embodiment comprises cyclosporin A, Tego Alkanol S2®, and Eucalyptus oil.

Another embodiment comprises cyclosporin A, Tego Alkanol S20 P®, and Eucalyptus oil.

Another embodiment comprises cyclosporin A, Triemulsifier 600 MS®, and Eucalyptus oil.

Another embodiment comprises cyclosporin A, a Glycerol Ester, and Eucalyptus oil.

Another embodiment comprises cyclosporin A, Agro #9 Wint SBO®, and Eucalyptus oil.

Another embodiment comprises cyclosporin A, Ahcovel Base 700®, and Eucalyptus oil.

Another embodiment comprises cyclosporin A, Aldo HMS FG®; and Eucalyptus oil.

Another embodiment comprises cyclosporin A, Aldo MLD®, and Eucalyptus oil.

Another embodiment comprises cyclosporin A, Aldo MLD FG®, and Eucalyptus oil.

Another embodiment comprises cyclosporin A, Aldo MO FG®, and Eucalyptus oil.

Another embodiment comprises cyclosporin A, Aldo MS®, and Eucalyptus oil.

Another embodiment comprises cyclosporin A, Aldo MS FG®, and Eucalyptus oil.

Another embodiment comprises cyclosporin A, Aldo MS LG FG®, and Eucalyptus oil.

Another embodiment comprises cyclosporin A, Aldo MSD®, and Eucalyptus oil.

Another embodiment comprises cyclosporin A, Aldo MSD FG®, and Eucalyptus oil.

Another embodiment comprises cyclosporin A, Aldosperse O-20 FG®, and Eucalyptus oil.

Another embodiment comprises cyclosporin A, Aldosperse TS-20 FG®, and Eucalyptus oil.

Another embodiment comprises cyclosporin A, Aldosperse TS-40 FG®, and Eucalyptus oil.

Another embodiment comprises cyclosporin A, Arlacel 165®, and Eucalyptus oil.

Another embodiment comprises cyclosporin A, Arlacel 186®, and Eucalyptus oil.

Another embodiment comprises cyclosporin A, Capmul GMO®, and Eucalyptus oil.

Another embodiment comprises cyclosporin A, Capmul GMS®, and Eucalyptus oil.

Another embodiment comprises cyclosporin A, Caprol 3GO®, and Eucalyptus oil.

Another embodiment comprises cyclosporin A, Caprol 3GVS®, and Eucalyptus oil.

Another embodiment comprises cyclosporin A, Caprol 6G2S®, and Eucalyptus oil.

Another embodiment comprises cyclosporin A, Caprol 10G40®, and Eucalyptus oil.

Another embodiment comprises cyclosporin A, Caprol 10G100®, and Eucalyptus oil.

Another embodiment comprises cyclosporin A, Caprol ET®, and Eucalyptus oil.

Another embodiment comprises cyclosporin A, Caprol PGE860®, and Eucalyptus oil.

Another embodiment comprises cyclosporin A, Cerasynt 945®, and Eucalyptus oil.

Another embodiment comprises cyclosporin A, Cerasynt GMS®, and Eucalyptus oil.

Another embodiment comprises cyclosporin A, Cerasynt Q®, and Eucalyptus oil.

Another embodiment comprises cyclosporin A, Cerasynt SD®, and Eucalyptus oil.

Another embodiment comprises cyclosporin A, Cerasynt WM®, and Eucalyptus oil.

Another embodiment comprises cyclosporin A, Chemsperse 14®, and Eucalyptus oil.

Another embodiment comprises cyclosporin A, Cremophor GO-32®, and Eucalyptus oil.

Another embodiment comprises cyclosporin A, Cremophor GS11®, and Eucalyptus oil.

Another embodiment comprises cyclosporin A, Cremophor GS-32®, and Eucalyptus oil.

Another embodiment comprises cyclosporin A, Cutina KD-16®, and Eucalyptus oil.

Another embodiment comprises cyclosporin A, Dehymuls PGPH®, and Eucalyptus oil.

Another embodiment comprises cyclosporin A, Dermol DGDIS®, and Eucalyptus oil.

Another embodiment comprises cyclosporin A, Dermol DGMIS®, and Eucalyptus oil.

Another embodiment comprises cyclosporin A, Dermol G-76®, and Eucalyptus oil.

Another embodiment comprises cyclosporin A, Dermol G-7DI®, and Eucalyptus oil.

Another embodiment comprises cyclosporin A, Dermol NGDI®, and Eucalyptus oil.

Another embodiment comprises cyclosporin A, Dermolan GLH®, and Eucalyptus oil.

Another embodiment comprises cyclosporin A, Drewmulse GMO®, and Eucalyptus oil.

Another embodiment comprises cyclosporin A, Drewpol 3-5-M®, and Eucalyptus oil.

Another embodiment comprises cyclosporin A, Durlac 100 W®, and Eucalyptus oil.

Another embodiment comprises cyclosporin A, Dur-Lo®, and Eucalyptus oil.

Another embodiment comprises cyclosporin A, Dynasan 118®, and Eucalyptus oil.

Another embodiment comprises cyclosporin A, EC-25®, and Eucalyptus oil.

Another embodiment comprises cyclosporin A, EM 40®, and Eucalyptus oil.

Another embodiment comprises cyclosporin A, Emerest 2400®, and Eucalyptus oil.

Another embodiment comprises cyclosporin A, Emerest 2452®, and Eucalyptus oil.

Another embodiment comprises cyclosporin A, Empilan G-26®, and Eucalyptus oil.

Another embodiment comprises cyclosporin A, Genapol TSM®, and Eucalyptus oil.

Another embodiment comprises cyclosporin A, Hostacerin DGI®, and Eucalyptus oil.

Another embodiment comprises cyclosporin A, Hostacerin DGL®, and Eucalyptus oil.

Another embodiment comprises cyclosporin A, Hostacerin DGMS®, and Eucalyptus oil.

Another embodiment comprises cyclosporin A, Hostacerin DGSB®, and Eucalyptus oil.

Another embodiment comprises cyclosporin A, Ice No. 2®, and Eucalyptus oil.

Another embodiment comprises cyclosporin A, Imwitor 742®, and Eucalyptus oil.

Another embodiment comprises cyclosporin A, Imwitor 780 K®, and Eucalyptus oil.

Another embodiment comprises cyclosporin A, Imwitor 960 Flakes®, and Eucalyptus oil.

Another embodiment comprises cyclosporin A, Isolan GI 34®, and Eucalyptus oil.

Another embodiment comprises cyclosporin A, Isolan GO 33®, and Eucalyptus oil.

Another embodiment comprises cyclosporin A, Kemester 1000®, and Eucalyptus oil.

Another embodiment comprises cyclosporin A, Kemester 2000®, and Eucalyptus oil.

Another embodiment comprises cyclosporin A, Kemester 2000®, and Eucalyptus oil.

Another embodiment comprises cyclosporin A, Kemester 6000SE®, and Eucalyptus oil.

Another embodiment comprises cyclosporin A, Lamecreme DGE 18®, and Eucalyptus oil.

Another embodiment comprises cyclosporin A, Lexemul 515®, and Eucalyptus oil.

Another embodiment comprises cyclosporin A, Lexemul 561®, and Eucalyptus oil.

Another embodiment comprises cyclosporin A, Lexemul AR®, and Eucalyptus oil.

Another embodiment comprises cyclosporin A, Lexemul AS®, and Eucalyptus oil.

Another embodiment comprises cyclosporin A, Lexemul GDL®, and Eucalyptus oil.

Another embodiment comprises cyclosporin A, Lexemul T®, and Eucalyptus oil.

Another embodiment comprises cyclosporin A, Lipomulse 165®, and Eucalyptus oil.

Another embodiment comprises cyclosporin A, Lumulse GML K®, and Eucalyptus oil.

Another embodiment comprises cyclosporin A, Lumulse GMO K®, and Eucalyptus oil.

Another embodiment comprises cyclosporin A, Lumulse GMR K®, and Eucalyptus oil.

Another embodiment comprises cyclosporin A, Lumulse GMT K®, and Eucalyptus oil.

Another embodiment comprises cyclosporin A, Magrabar GMC®, and Eucalyptus oil.

Another embodiment comprises cyclosporin A, Magrabar GMO-CK®, and Eucalyptus oil.

Another embodiment comprises cyclosporin A, Magrabar MDG-5050®, and Eucalyptus oil.

Another embodiment comprises cyclosporin A, Magrabar PGO-315®, and Eucalyptus oil.

Another embodiment comprises cyclosporin A, Magrabar PGO-1010®, and Eucalyptus oil.

Another embodiment comprises cyclosporin A, Mazol 300K®, and Eucalyptus oil.

Another embodiment comprises cyclosporin A, Mazol GMO-K®, and Eucalyptus oil.

Another embodiment comprises cyclosporin A, Mazol GMS-K®, and Eucalyptus oil.

Another embodiment comprises cyclosporin A, Mazol PG031-K®, and Eucalyptus oil.

Another embodiment comprises cyclosporin A, Miglyol 812®, and Eucalyptus oil.

Another embodiment comprises cyclosporin A, Norfox 165C®, and Eucalyptus oil.

Another embodiment comprises cyclosporin A, Schercemol GMIS®, and Eucalyptus oil.

Another embodiment comprises cyclosporin A, Tegin®, and Eucalyptus oil.

Another embodiment comprises cyclosporin A, Tegin 4100 Pellets®, and Eucalyptus oil.

Another embodiment comprises cyclosporin A, Tegin M Pellets®, and Eucalyptus oil.

Another embodiment comprises cyclosporin A, Tegin OV®, and Eucalyptus oil.

Another embodiment comprises cyclosporin A, Teginacid H®, and Eucalyptus oil.

Another embodiment comprises cyclosporin A, Tego Cosmo P813®, and Eucalyptus oil.

Another embodiment comprises cyclosporin A, Wickenol 535®, and Eucalyptus oil.

Another embodiment comprises cyclosporin A, Witconol 14®, and Eucalyptus oil.

Another embodiment comprises cyclosporin A, Witconol 14®, and Eucalyptus oil.

Another embodiment comprises cyclosporin A, Witconol 14F®, and Eucalyptus oil.

Another embodiment comprises cyclosporin A, Witconol 18L®, and Eucalyptus oil.

Another embodiment comprises cyclosporin A, Witconol GOT®, and Eucalyptus oil.

Another embodiment comprises cyclosporin A, Witconol MST®, and Eucalyptus oil.

Another embodiment comprises cyclosporin A, Witconol RHT®, and Eucalyptus oil.

Another embodiment comprises cyclosporin A, a Glycol Ester, and Eucalyptus oil.

Another embodiment comprises cyclosporin A, Alkamuls 600 DO®, and Eucalyptus oil.

Another embodiment comprises cyclosporin A, Alkamuls SEG®, and Eucalyptus oil.

Another embodiment comprises cyclosporin A, Atlas EM-2®, and Eucalyptus oil.

Another embodiment comprises cyclosporin A, Cerasynt IP®, and Eucalyptus oil.

Another embodiment comprises cyclosporin A, Cerasynt M®, and Eucalyptus oil.

Another embodiment comprises cyclosporin A, Cerasynt MN®, and Eucalyptus oil.

Another embodiment comprises cyclosporin A, Cerasynt PA®, and Eucalyptus oil.

Another embodiment comprises cyclosporin A, Chemsperse EGDS®, and Eucalyptus oil.

Another embodiment comprises cyclosporin A, Chemsperse EGMS®, and Eucalyptus oil.

Another embodiment comprises cyclosporin A, Colonial Monolaurin®, and Eucalyptus oil.

Another embodiment comprises cyclosporin A, DeMuls SGE-95®, and Eucalyptus oil.

Another embodiment comprises cyclosporin A, Eccoterge 200®, and Eucalyptus oil.

Another embodiment comprises cyclosporin A, Emerest 2380®, and Eucalyptus oil.

Another embodiment comprises cyclosporin A, Ethox 2610®, and Eucalyptus oil.

Another embodiment comprises cyclosporin A, Ethox DO-9®, and Eucalyptus oil.

Another embodiment comprises cyclosporin A, Ethox DO-14®, and Eucalyptus oil.

Another embodiment comprises cyclosporin A, Ethox SO-9®, and Eucalyptus oil.

Another embodiment comprises cyclosporin A, Fizul MD-318®, and Eucalyptus oil.

Another embodiment comprises cyclosporin A, Genapol EGDS-VHP®, and Eucalyptus oil.

Another embodiment comprises cyclosporin A, Genapol TS Powder®, and Eucalyptus oil.

Another embodiment comprises cyclosporin A, Hostacerin WO®, and Eucalyptus oil.

Another embodiment comprises cyclosporin A, Inversol 140®, and Eucalyptus oil.

Another embodiment comprises cyclosporin A, Kemester 104®, and Eucalyptus oil.

Another embodiment comprises cyclosporin A, Kemester 205®, and Eucalyptus oil.

Another embodiment comprises cyclosporin A, Kemester 226®, and Eucalyptus oil.

Another embodiment comprises cyclosporin A, Kemester 5221SE®, and Eucalyptus oil.

Another embodiment comprises cyclosporin A, Kemester EGDS®, and Eucalyptus oil.

Another embodiment comprises cyclosporin A, Lexemul EGDS®, and Eucalyptus oil.

Another embodiment comprises cyclosporin A, Lexemul EGMS®, and Eucalyptus oil.

Another embodiment comprises cyclosporin A, Lexemul P®, and Eucalyptus oil.

Another embodiment comprises cyclosporin A, Lipo DGLS®, and Eucalyptus oil.

Another embodiment comprises cyclosporin A, Lipo EGDS®, and Eucalyptus oil.

Another embodiment comprises cyclosporin A, Lipo PGMS®, and Eucalyptus oil.

Another embodiment comprises cyclosporin A, Liposorb S-4®, and Eucalyptus oil.

Another embodiment comprises cyclosporin A, Liposorb TO-20®, and Eucalyptus oil.

Another embodiment comprises cyclosporin A, Lumulse PGO®, and Eucalyptus oil.

Another embodiment comprises cyclosporin A, Mackester EGDS®, and Eucalyptus oil.

Another embodiment comprises cyclosporin A, Mackester EGMS®, and Eucalyptus oil.

Another embodiment comprises cyclosporin A, Mackester GSTP®, and Eucalyptus oil.

Another embodiment comprises cyclosporin A, Mackester Series®, and Eucalyptus oil.

Another embodiment comprises cyclosporin A, Magrabar PDG-50®, and Eucalyptus oil.

Another embodiment comprises cyclosporin A, Mapeg 6000 DS®, and Eucalyptus oil.

Another embodiment comprises cyclosporin A, Marlowet 4702®, and Eucalyptus oil.

Another embodiment comprises cyclosporin A, Monalube 305®, and Eucalyptus oil.

Another embodiment comprises cyclosporin A, Monalube 310®, and Eucalyptus oil.

Another embodiment comprises cyclosporin A, Monalube 315®, and Eucalyptus oil.

Another embodiment comprises cyclosporin A, Monalube 320®, and Eucalyptus oil.

Another embodiment comprises cyclosporin A, Monalube 325®, and Eucalyptus oil.

Another embodiment comprises cyclosporin A, Monalube 330®, and Eucalyptus oil.

Another embodiment comprises cyclosporin A, Naturechem PGHS®, and Eucalyptus oil.

Another embodiment comprises cyclosporin A, Polycastorol PLO-840®, and Eucalyptus oil.

Another embodiment comprises cyclosporin A, Polytex 10M®, and Eucalyptus oil.

Another embodiment comprises cyclosporin A, Ritasynt IP®, and Eucalyptus oil.

Another embodiment comprises cyclosporin A, Ross Chem PEG 600 DT®, and Eucalyptus oil.

Another embodiment comprises cyclosporin A, Schercemol PGMS®, and Eucalyptus oil.

Another embodiment comprises cyclosporin A, Spontd H-44C®, and Eucalyptus oil.

Another embodiment comprises cyclosporin A, Tegin G®, and Eucalyptus oil.

Another embodiment comprises cyclosporin A, Witbreak DGE-182®, and Eucalyptus oil.

Another embodiment comprises cyclosporin A, Witbreak DGE-182®, and Eucalyptus oil.

Another embodiment comprises cyclosporin A, Witbreak DRA-21®, and Eucalyptus oil.

Another embodiment comprises cyclosporin A, Witbreak DRA-21®, and Eucalyptus oil.

Another embodiment comprises cyclosporin A, Witbreak DRA-50®, and Eucalyptus oil.

Another embodiment comprises cyclosporin A, Witbreak DRA-50®, and Eucalyptus oil.

Another embodiment comprises cyclosporin A, Witconol F26-46®, and Eucalyptus oil.

Another embodiment comprises cyclosporin A, Witconol H-32®, and Eucalyptus oil.

Another embodiment comprises cyclosporin A, Witconol H-33®, and Eucalyptus oil.

Another embodiment comprises cyclosporin A, Witconol H-35A®, and Eucalyptus oil.

Another embodiment comprises cyclosporin A, Witconol RHP®, and Eucalyptus oil.

Another embodiment comprises cyclosporin A, a Lanolin-based Derivative, and Eucalyptus oil.

Another embodiment comprises cyclosporin A, Amerchol CAB®, and Eucalyptus oil.

Another embodiment comprises cyclosporin A, Amerchol L-101®, and Eucalyptus oil.

Another embodiment comprises cyclosporin A, Amerlate LFA-LO®, and Eucalyptus oil.

Another embodiment comprises cyclosporin A, Amerlate P®, and Eucalyptus oil.

Another embodiment comprises cyclosporin A, Barre Common Degras®, and Eucalyptus oil.

Another embodiment comprises cyclosporin A, Cholesterol NF®, and Eucalyptus oil.

Another embodiment comprises cyclosporin A, Crodalan AWS®, and Eucalyptus oil.

Another embodiment comprises cyclosporin A, Crodalan LA®, and Eucalyptus oil.

Another embodiment comprises cyclosporin A, Emery 1650®, and Eucalyptus. oil.

Another embodiment comprises cyclosporin A, Emery 1650®, and Eucalyptus oil.

Another embodiment comprises cyclosporin A, Emery 1740®, and Eucalyptus oil.

Another embodiment comprises cyclosporin A, Emery 1740®, and Eucalyptus oil.

Another embodiment comprises cyclosporin A, Forlan 500®, and Eucalyptus oil.

Another embodiment comprises cyclosporin A, Forlan L®, and Eucalyptus oil.

Another embodiment comprises cyclosporin A, Laneto 50®, and Eucalyptus oil.

Another embodiment comprises cyclosporin A, Laneto 100®, and Eucalyptus oil.

Another embodiment comprises cyclosporin A, Laneto AWS®, and Eucalyptus oil.

Another embodiment comprises cyclosporin A, Lanfrax 1776®, and Eucalyptus oil.

Another embodiment comprises cyclosporin A, Lanfrax 1776®, and Eucalyptus oil.

Another embodiment comprises cyclosporin A, Lanogel 21®, and Eucalyptus oil.

Another embodiment comprises cyclosporin A, Lipolan®, and Eucalyptus oil.

Another embodiment comprises cyclosporin A, Lipolan 31®, and Eucalyptus oil.

Another embodiment comprises cyclosporin A, OHlan®, and Eucalyptus oil.

Another embodiment comprises cyclosporin A, Polychol 5®, and Eucalyptus oil.

Another embodiment comprises cyclosporin A, Polychol 15®, and Eucalyptus oil.

Another embodiment comprises cyclosporin A, Ritacetyl®, and Eucalyptus oil.

Another embodiment comprises cyclosporin A, Ritachol®, and Eucalyptus oil.

Another embodiment comprises cyclosporin A, Ritahydrox®, and Eucalyptus oil.

Another embodiment comprises cyclosporin A, Ritalafa®, and Eucalyptus oil.

Another embodiment comprises cyclosporin A, Ritalan®, and Eucalyptus oil.

Another embodiment comprises cyclosporin A, Ritalan AWS®, and Eucalyptus oil.

Another embodiment comprises cyclosporin A, Ritalan C®, and Eucalyptus oil.

Another embodiment comprises cyclosporin A, Ritawax®, and Eucalyptus oil.

Another embodiment comprises cyclosporin A, Ritawax AEO®, and Eucalyptus oil.

Another embodiment comprises cyclosporin A, Ritawax ALA®, and Eucalyptus oil.

Another embodiment comprises cyclosporin A, Solan/Solan 50/Super Solan®, and Eucalyptus oil.

Another embodiment comprises cyclosporin A, Super Hartolan/Hartolan®, and Eucalyptus oil.

Another embodiment comprises cyclosporin A, Supersat AWS-4®, and Eucalyptus oil.

Another embodiment comprises cyclosporin A, Supersat AWS-24®, and Eucalyptus oil.

Another embodiment comprises cyclosporin A, a Lecithin or Lecithin Derivative, and Eucalyptus oil.

Another embodiment comprises cyclosporin A, Alcolec®, and Eucalyptus oil.

Another embodiment comprises cyclosporin A, Lecithin®, and Eucalyptus oil.

Another embodiment comprises cyclosporin A, Lexin K®, and Eucalyptus oil.

Another embodiment comprises cyclosporin A, Natipide®, and Eucalyptus oil.

Another embodiment comprises cyclosporin A, a Lignin or Lignin Derivative, and Eucalyptus oil.

Another embodiment comprises cyclosporin A, Diwatex XP 9®, and Eucalyptus oil.

Another embodiment comprises cyclosporin A, Dynasperse LCD®, and Eucalyptus oil.

Another embodiment comprises cyclosporin A, Indulin SAL®, and Eucalyptus oil.

Another embodiment comprises cyclosporin A, Indulin W-1®, and Eucalyptus oil.

Another embodiment comprises cyclosporin A, Indulin W-5®, and Eucalyptus oil.

Another embodiment comprises cyclosporin A, Lignosol FTA®, and Eucalyptus oil.

Another embodiment comprises cyclosporin A, Lignosol SFX-65®, and Eucalyptus oil.

Another embodiment comprises cyclosporin A, Marasperse 52 CP®, and Eucalyptus oil.

Another embodiment comprises cyclosporin A, Marasperse AG®, and Eucalyptus oil.

Another embodiment comprises cyclosporin A, Marasperse CBOS-4®, and Eucalyptus oil.

Another embodiment comprises cyclosporin A, Ufoxane 2®, and Eucalyptus oil.

Another embodiment comprises cyclosporin A, a Methyl Ester, and Eucalyptus oil.

Another embodiment comprises cyclosporin A, E.B. Cleaner AK®, and Eucalyptus oil.

Another embodiment comprises cyclosporin A, Oleocal ME-70®, and Eucalyptus oil.

Another embodiment comprises cyclosporin A, Trade Name Company®, and Eucalyptus oil.

Another embodiment comprises cyclosporin A, Methyl Esters (cont'd)®, and Eucalyptus oil.

Another embodiment comprises cyclosporin A, Oleocal ME-92®, and Eucalyptus oil.

Another embodiment comprises cyclosporin A, Oleocal ME-112®, and Eucalyptus oil.

Another embodiment comprises cyclosporin A, Oleocal ME-130®, and Eucalyptus oil.

Another embodiment comprises cyclosporin A, a Monoglyceride or a Derivative thereof, and Eucalyptus oil.

Another embodiment comprises cyclosporin A, Dynacet 211®, and Eucalyptus oil.

Another embodiment comprises cyclosporin A, Hetsorb S-20®, and Eucalyptus oil.

Another embodiment comprises cyclosporin A, Imwitor 191®, and Eucalyptus oil.

Another embodiment comprises cyclosporin A, Imwitor 370®, and Eucalyptus oil.

Another embodiment comprises cyclosporin A, Imwitor 375®, and Eucalyptus oil.

Another embodiment comprises cyclosporin A, Imwitor 900®, and Eucalyptus oil.

Another embodiment comprises cyclosporin A, Imwitor 945®, and Eucalyptus oil.

Another embodiment comprises cyclosporin A, Imwitor 2020®, and Eucalyptus oil.

Another embodiment comprises cyclosporin A, Kemester 5500®, and Eucalyptus oil.

Another embodiment comprises cyclosporin A, Kemester 6000®, and Eucalyptus oil.

Another embodiment comprises cyclosporin A, Magrabar GMC®, and Eucalyptus oil.

Another embodiment comprises cyclosporin A, Magrabar GMO-CK®, and Eucalyptus oil.

Another embodiment comprises cyclosporin A, Magrabar GPC-10®, and Eucalyptus oil.

Another embodiment comprises cyclosporin A, Magrabar MDG-5050®, and Eucalyptus oil.

Another embodiment comprises cyclosporin A, Monalube 335®, and Eucalyptus oil.

Another embodiment comprises cyclosporin A, Monoglycerides Glyceryl Monestearate Archer Daniels Midland Company®, and Eucalyptus oil.

Another embodiment comprises cyclosporin A, Rita GMS®, and Eucalyptus oil.

Another embodiment comprises cyclosporin A, Ritamulse SCG®, and Eucalyptus oil.

Another embodiment comprises cyclosporin A, Softigen 701®, and Eucalyptus oil.

Another embodiment comprises cyclosporin A, Tally 100 Plus®, and Eucalyptus oil.

Another embodiment comprises cyclosporin A, a Polyethylene Glycol, and Eucalyptus oil.

Another embodiment comprises cyclosporin A, Emulgade PL 68/50®, and Eucalyptus oil.

Another embodiment comprises cyclosporin A, Lumulse PEG®, and Eucalyptus oil.

Another embodiment comprises cyclosporin A, Rhodasun PEG-400®, and Eucalyptus oil.

Another embodiment comprises cyclosporin A, Rhodasurf PEG-600®, and Eucalyptus oil.

Another embodiment comprises cyclosporin A, Witconol PEG-400®, and Eucalyptus oil.

Another embodiment comprises cyclosporin A, a Polymeric Surfactant, and Eucalyptus oil.

Another embodiment comprises cyclosporin A, Acritamer PNC-EG®, and Eucalyptus oil.

Another embodiment comprises cyclosporin A, Ag-Rho DEP-775®, and Eucalyptus oil.

Another embodiment comprises cyclosporin A, APG 325N Glycoside®, and Eucalyptus oil.

Another embodiment comprises cyclosporin A, Aristoflex AVC®, and Eucalyptus oil.

Another embodiment comprises cyclosporin A, Aristoflex HMB®, and Eucalyptus oil.

Another embodiment comprises cyclosporin A, Burco NPS-225®, and Eucalyptus oil.

Another embodiment comprises cyclosporin A, Burco NPS-816®, and Eucalyptus oil.

Another embodiment comprises cyclosporin A, Chemccinate 5603®, and Eucalyptus oil.

Another embodiment comprises cyclosporin A, Cosmedia Guar C-261N®, and Eucalyptus oil.

Another embodiment comprises cyclosporin A, Gantrez S-95®, and Eucalyptus oil.

Another embodiment comprises cyclosporin A, Glucopon 220 UP®, and Eucalyptus oil.

Another embodiment comprises cyclosporin A, Glucopon 225 DK®, and Eucalyptus oil.

Another embodiment comprises cyclosporin A, Glucopon 425 N®, and Eucalyptus oil.

Another embodiment comprises cyclosporin A, Glucopon 600 UP®, and Eucalyptus oil.

Another embodiment comprises cyclosporin A, Glucopon 625 UP®, and Eucalyptus oil.

Another embodiment comprises cyclosporin A, Pemulen 1621®, and Eucalyptus oil.

Another embodiment comprises cyclosporin A, Pemulen 1622®, and Eucalyptus oil.

Another embodiment comprises cyclosporin A, Pemulen TR-1®, and Eucalyptus oil.

Another embodiment comprises cyclosporin A, Pemulen TR-2®, and Eucalyptus oil.

Another embodiment comprises cyclosporin A, Plantacare 818®, and Eucalyptus oil.

Another embodiment comprises cyclosporin A, Plantapon LGC Sorb®, and Eucalyptus oil.

Another embodiment comprises cyclosporin A, Plantaren 1200N®, and Eucalyptus oil.

Another embodiment comprises cyclosporin A, Plantaren 2000N®, and Eucalyptus oil.

Another embodiment comprises cyclosporin A, Viscolam AT 64®, and Eucalyptus oil.

Another embodiment comprises cyclosporin A, Viscolam AT 64P®, and Eucalyptus oil.

Another embodiment comprises cyclosporin A, Viscolam AT 100®, and Eucalyptus oil.

Another embodiment comprises cyclosporin A, Viscolam MAC 7®, and Eucalyptus oil.

Another embodiment comprises cyclosporin A, Viscolam SMC 20®, and Eucalyptus oil.

Another embodiment comprises cyclosporin A, Witbreak RTC-323®, and Eucalyptus oil.

Another embodiment comprises cyclosporin A, WSI 3700®, and Eucalyptus oil.

Another embodiment comprises cyclosporin A, a Propoxylated or Ethoxylated Fatty Acid, Alcohol, or Alkyl Phenol, and Eucalyptus oil.

Another embodiment comprises cyclosporin A, Antarox AA-60®, and Eucalyptus oil.

Another embodiment comprises cyclosporin A, Antarox LF-224®, and Eucalyptus oil.

Another embodiment comprises cyclosporin A, Burcomul DFE-45®, and Eucalyptus oil.

Another embodiment comprises cyclosporin A, Burcoterge LFE-1000®, and Eucalyptus oil.

Another embodiment comprises cyclosporin A, Chemal LF-25B®, and Eucalyptus oil.

Another embodiment comprises cyclosporin A, Chemal LF-40B®, and Eucalyptus oil.

Another embodiment comprises cyclosporin A, Dehypon LS-36®, and Eucalyptus oil.

Another embodiment comprises cyclosporin A, Dehypon LS-36®, and Eucalyptus oil.

Another embodiment comprises cyclosporin A, Dehypon LS-54®, and Eucalyptus oil.

Another embodiment comprises cyclosporin A, Dehypon LS-54®, and Eucalyptus oil.

Another embodiment comprises cyclosporin A, Delonic 100 VLF®, and Eucalyptus oil.

Another embodiment comprises cyclosporin A, Delonic LF-60 MOD®, and Eucalyptus oil.

Another embodiment comprises cyclosporin A, Empiderm B®, and Eucalyptus oil.

Another embodiment comprises cyclosporin A, Ethylan 1206®, and Eucalyptus oil.

Another embodiment comprises cyclosporin A, Ethylan NS-500K®, and Eucalyptus oil.

Another embodiment comprises cyclosporin A, Ethylan NS-500LQ®, and Eucalyptus oil.

Another embodiment comprises cyclosporin A, Genapol 1392®, and Eucalyptus oil.

Another embodiment comprises cyclosporin A, Genapol 2317®, and Eucalyptus oil.

Another embodiment comprises cyclosporin A, Genapol 26EP710®, and Eucalyptus oil.

Another embodiment comprises cyclosporin A, Genapol EP 1022®, and Eucalyptus oil.

Another embodiment comprises cyclosporin A, Genapol EP 1024®, and Eucalyptus oil.

Another embodiment comprises cyclosporin A, Genapol EP 6068®, and Eucalyptus oil.

Another embodiment comprises cyclosporin A, Genapol NP915®, and Eucalyptus oil.

Another embodiment comprises cyclosporin A, Kieralon MFB®, and Eucalyptus oil.

Another embodiment comprises cyclosporin A, Lumisolve CSA-80 V®, and Eucalyptus oil.

Another embodiment comprises cyclosporin A, Marlowet 5001®, and Eucalyptus oil.

Another embodiment comprises cyclosporin A, Marlox FK 64®, and Eucalyptus oil.

Another embodiment comprises cyclosporin A, Marlox MO 124®, and Eucalyptus oil.

Another embodiment comprises cyclosporin A, Marlox S 58®, and Eucalyptus oil.

Another embodiment comprises cyclosporin A, Nonatell 1003®, and Eucalyptus oil.

Another embodiment comprises cyclosporin A, Nonatell 1038®, and Eucalyptus oil.

Another embodiment comprises cyclosporin A, Nonatell 1052®, and Eucalyptus oil.

Another embodiment comprises cyclosporin A, Nonatell 1061®, and Eucalyptus oil.

Another embodiment comprises cyclosporin A, Nonatell 1075®, and Eucalyptus oil.

Another embodiment comprises cyclosporin A, Nonatell 1088®, and Eucalyptus oil.

Another embodiment comprises cyclosporin A, Nonatell 1123®, and Eucalyptus oil.

Another embodiment comprises cyclosporin A, Nonatell 1153®, and Eucalyptus oil.

Another embodiment comprises cyclosporin A, Nonatell 1161®, and Eucalyptus oil.

Another embodiment comprises cyclosporin A, Nonatell 1172®, and Eucalyptus oil.

Another embodiment comprises cyclosporin A, Nonatell 1181®, and Eucalyptus oil.

Another embodiment comprises cyclosporin A, Nortox 36®, and Eucalyptus oil.

Another embodiment comprises cyclosporin A, Procetyl AWS®, and Eucalyptus oil.

Another embodiment comprises cyclosporin A, Sandoxylate SX 412®, and Eucalyptus oil.

Another embodiment comprises cyclosporin A, Sandoxylate SX 418®, and Eucalyptus oil.

Another embodiment comprises cyclosporin A, Surfonic JL-80X®, and Eucalyptus oil.

Another embodiment comprises cyclosporin A, Surfonic JL-80X-B1®, and Eucalyptus oil.

Another embodiment comprises cyclosporin A, Surfonic L4-29X®, and Eucalyptus oil.

Another embodiment comprises cyclosporin A, Surfonic LF®, and Eucalyptus oil.

Another embodiment comprises cyclosporin A, T-Det A826®, and Eucalyptus oil.

Another embodiment comprises cyclosporin A, T-Det LF-416®, and Eucalyptus oil.

Another embodiment comprises cyclosporin A, Tergitol Min-Foam 1X®, and Eucalyptus oil.

Another embodiment comprises cyclosporin A, Tergitol Min-Foam 2X®, and Eucalyptus oil.

Another embodiment comprises cyclosporin A, Triton CF-21®, and Eucalyptus oil.

Another embodiment comprises cyclosporin A, Triton CF-76®, and Eucalyptus oil.

Another embodiment comprises cyclosporin A, Triton XL-80N®, and Eucalyptus oil.

Another embodiment comprises cyclosporin A, Witconol NS-98®, and Eucalyptus oil.

Another embodiment comprises cyclosporin A, Witconol NS-108LQ®, and Eucalyptus oil.

Another embodiment comprises cyclosporin A, Witconol NS-145®, and Eucalyptus oil.

Another embodiment comprises cyclosporin A, Witconol NS-179®, and Eucalyptus oil.

Another embodiment comprises cyclosporin A, a Protein-based Surfactant, and Eucalyptus oil.

Another embodiment comprises cyclosporin A, AminoFoam W®, and Eucalyptus oil.

Another embodiment comprises cyclosporin A, Amiter LGOD-2®, and Eucalyptus oil.

Another embodiment comprises cyclosporin A, Amiter LGS-2®, and Eucalyptus oil.

Another embodiment comprises cyclosporin A, Amiter LGS-5®, and Eucalyptus oil.

Another embodiment comprises cyclosporin A, Lamepon S®, and Eucalyptus oil.

Another embodiment comprises cyclosporin A, Lamepon S®, and Eucalyptus oil.

Another embodiment comprises cyclosporin A, Maypon 4C®, and Eucalyptus oil.

Another embodiment comprises cyclosporin A, May-Tein C®, and Eucalyptus oil.

Another embodiment comprises cyclosporin A, May-Tein CT®, and Eucalyptus oil.

Another embodiment comprises cyclosporin A, May-Tein KTS®, and Eucalyptus oil.

Another embodiment comprises cyclosporin A, May-Tein SY®, and Eucalyptus oil.

Another embodiment comprises cyclosporin A, Plantapon S®, and Eucalyptus oil.

Another embodiment comprises cyclosporin A, Proteol APL®, and Eucalyptus oil.

Another embodiment comprises cyclosporin A, Proteol OAT®, and Eucalyptus oil.

Another embodiment comprises cyclosporin A, Pyroter CPI-40®, and Eucalyptus oil.

Another embodiment comprises cyclosporin A, Pyroter GPI-25®, and Eucalyptus oil.

Another embodiment comprises cyclosporin A, Supro-Tein S®, and Eucalyptus oil.

Another embodiment comprises cyclosporin A, Supro-Tein V®, and Eucalyptus oil.

Another embodiment comprises cyclosporin A,® and Eucalyptus oil.

Another embodiment comprises cyclosporin A, a Sarcosine Derivative®, and Eucalyptus oil.

Another embodiment comprises cyclosporin A, Crodasinic LS-30®, and Eucalyptus oil.

Another embodiment comprises cyclosporin A, Vanseal CS®, and Eucalyptus oil.

Another embodiment comprises cyclosporin A, Vanseal LS®, and Eucalyptus oil.

Another embodiment comprises cyclosporin A, Vanseal MS®, and Eucalyptus oil.

Another embodiment comprises cyclosporin A, Vanseal NACS-30®, and Eucalyptus oil.

Another embodiment comprises cyclosporin A, Vanseal NALS-95®, and Eucalyptus oil.

Another embodiment comprises cyclosporin A, Vanseal OS®, and Eucalyptus oil.

Another embodiment comprises cyclosporin A,® and Eucalyptus oil.

Another embodiment comprises cyclosporin A, a Silicone-based Surfactant®, and Eucalyptus oil.

Another embodiment comprises cyclosporin A, Abil-B-9950®, and Eucalyptus oil.

Another embodiment comprises cyclosporin A, Abil Care 85®, and Eucalyptus oil.

Another embodiment comprises cyclosporin A, Abil EM 90®, and Eucalyptus oil.

Another embodiment comprises cyclosporin A, Abil EM 97®, and Eucalyptus oil.

Another embodiment comprises cyclosporin A, Abil WE-09®, and Eucalyptus oil.

Another embodiment comprises cyclosporin A, Dow Corning 1248 Fluid®, and Eucalyptus oil.

Another embodiment comprises cyclosporin A, Dow Corning 3225C®, and Eucalyptus oil.

Another embodiment comprises cyclosporin A, Dow Corning 5200®, and Eucalyptus oil.

Another embodiment comprises cyclosporin A, Dow Corning Q4-3667®, and Eucalyptus oil.

Another embodiment comprises cyclosporin A, Monasil PCA®, and Eucalyptus oil.

Another embodiment comprises cyclosporin A, Monasil PDM®, and Eucalyptus oil.

Another embodiment comprises cyclosporin A, Monasil PLN®, and Eucalyptus oil.

Another embodiment comprises cyclosporin A, Polyderm PPI-SI-WS®, and Eucalyptus oil.

Another embodiment comprises cyclosporin A, Troysol 380W®, and Eucalyptus oil.

Another embodiment comprises cyclosporin A, Troysol S366®, and Eucalyptus oil.

Another embodiment comprises cyclosporin A, a Sorbitan Derivative, and Eucalyptus oil.

Another embodiment comprises cyclosporin A, Alkamuls SML®, and Eucalyptus oil.

Another embodiment comprises cyclosporin A, Alkamuls SMO®, and Eucalyptus oil.

Another embodiment comprises cyclosporin A, Alkamuls STO®, and Eucalyptus oil.

Another embodiment comprises cyclosporin A, Arlacel 20®, and Eucalyptus oil.

Another embodiment comprises cyclosporin A, Arlacel 40®, and Eucalyptus oil.

Another embodiment comprises cyclosporin A, Arlacel 60®, and Eucalyptus oil.

Another embodiment comprises cyclosporin A, Arlacel 80®, and Eucalyptus oil.

Another embodiment comprises cyclosporin A, Arlacel C®, and Eucalyptus oil.

Another embodiment comprises cyclosporin A, Armul 21®, and Eucalyptus oil.

Another embodiment comprises cyclosporin A, Atlox 80®, and Eucalyptus oil.

Another embodiment comprises cyclosporin A, Atlox 847®, and Eucalyptus oil.

Another embodiment comprises cyclosporin A, Atlox 1045A®, and Eucalyptus oil.

Another embodiment comprises cyclosporin A, Canarcel 20®, and Eucalyptus oil.

Another embodiment comprises cyclosporin A, Canarcel 60®, and Eucalyptus oil.

Another embodiment comprises cyclosporin A, Canarcel 80®, and Eucalyptus oil.

Another embodiment comprises cyclosporin A, Canarcel TW 20®, and Eucalyptus oil.

Another embodiment comprises cyclosporin A, Canarcel TW 60®, and Eucalyptus oil.

Another embodiment comprises cyclosporin A, Canarcel TW 80®, and Eucalyptus oil.

Another embodiment comprises cyclosporin A, Coladet BSB-P®, and Eucalyptus oil.

Another embodiment comprises cyclosporin A, Customulse O-20®, and Eucalyptus oil.

Another embodiment comprises cyclosporin A, Dehymuls E®, and Eucalyptus oil.

Another embodiment comprises cyclosporin A, DeSotan SMO®, and Eucalyptus oil.

Another embodiment comprises cyclosporin A, DeSotan SMO-20®, and Eucalyptus oil.

Another embodiment comprises cyclosporin A, DeSotan SMT®, and Eucalyptus oil.

Another embodiment comprises cyclosporin A, DeSotan SMT-20®, and Eucalyptus oil.

Another embodiment comprises cyclosporin A, Durfax 60®, and Eucalyptus oil.

Another embodiment comprises cyclosporin A, Durfax 65®, and Eucalyptus oil.

Another embodiment comprises cyclosporin A, Durfax 80®, and Eucalyptus oil.

Another embodiment comprises cyclosporin A, Durtan 60®, and Eucalyptus oil.

Another embodiment comprises cyclosporin A, Durtan 65®, and Eucalyptus oil.

Another embodiment comprises cyclosporin A, Liposorb L®, and Eucalyptus oil.

Another embodiment comprises cyclosporin A, Liposorb L-10®, and Eucalyptus oil.

Another embodiment comprises cyclosporin A, Liposorb L-20®, and Eucalyptus oil.

Another embodiment comprises cyclosporin A, Liposorb O®, and Eucalyptus oil.

Another embodiment comprises cyclosporin A, Liposorb O-20®, and Eucalyptus oil.

Another embodiment comprises cyclosporin A, Liposorb P®, and Eucalyptus oil.

Another embodiment comprises cyclosporin A, Liposorb P-20®, and Eucalyptus oil.

Another embodiment comprises cyclosporin A, Liposorb S®, and Eucalyptus oil.

Another embodiment comprises cyclosporin A, Liposorb S-20®, and Eucalyptus oil.

Another embodiment comprises cyclosporin A, Liposorb SQO®, and Eucalyptus oil.

Another embodiment comprises cyclosporin A, Liposorb TO®, and Eucalyptus oil.

Another embodiment comprises cyclosporin A, Liposorb TS®, and Eucalyptus oil.

Another embodiment comprises cyclosporin A, Liposorb TS-20®, and Eucalyptus oil.

Another embodiment comprises cyclosporin A, Lumisorb PS®, and Eucalyptus oil.

Another embodiment comprises cyclosporin A, Lumisorb SMO (T)®, and Eucalyptus oil.

Another embodiment comprises cyclosporin A, Lumisorb SMS K®, and Eucalyptus oil.

Another embodiment comprises cyclosporin A, Lumisorb SSO®, and Eucalyptus oil.

Another embodiment comprises cyclosporin A, Lumisorb STS K®, and Eucalyptus oil.

Another embodiment comprises cyclosporin A, Lumisorb STT®, and Eucalyptus oil.

Another embodiment comprises cyclosporin A, Magrabar SMO®, and Eucalyptus oil.

Another embodiment comprises cyclosporin A, Magrabar SMO-VEG®, and Eucalyptus oil.

Another embodiment comprises cyclosporin A, Magrabar SMT®, and Eucalyptus oil.

Another embodiment comprises cyclosporin A, Magrabar STO®, and Eucalyptus oil.

Another embodiment comprises cyclosporin A, Miracare BC-27®, and Eucalyptus oil.

Another embodiment comprises cyclosporin A, Ritabate 20®, and Eucalyptus oil.

Another embodiment comprises cyclosporin A, Ritabate 40®, and Eucalyptus oil.

Another embodiment comprises cyclosporin A, Ritabate 60®, and Eucalyptus oil.

Another embodiment comprises cyclosporin A, Ritabate 80®, and Eucalyptus oil.

Another embodiment comprises cyclosporin A, T-Maz®, and Eucalyptus oil.

Another embodiment comprises cyclosporin A, Tego SML®, and Eucalyptus oil.

Another embodiment comprises cyclosporin A, Tego SML 20®, and Eucalyptus oil.

Another embodiment comprises cyclosporin A, Tego SMO 80 V®, and Eucalyptus oil.

Another embodiment comprises cyclosporin A, Tego SMO V®, and Eucalyptus oil.

Another embodiment comprises cyclosporin A, Tego SMS®, and Eucalyptus oil.

Another embodiment comprises cyclosporin A, Tego STO V®, and Eucalyptus oil.

Another embodiment comprises cyclosporin A, Tween 21®, and Eucalyptus oil.

Another embodiment comprises cyclosporin A, Tween 40®, and Eucalyptus oil.

Another embodiment comprises cyclosporin A, Tween 60®, and Eucalyptus oil.

Another embodiment comprises cyclosporin A, Tween 60 K®, and Eucalyptus oil.

Another embodiment comprises cyclosporin A, Tween 61®, and Eucalyptus oil.

Another embodiment comprises cyclosporin A, Tween 65®, and Eucalyptus oil.

Another embodiment comprises cyclosporin A, Tween 80®, and Eucalyptus oil.

Another embodiment comprises cyclosporin A, Tween 80 K®, and Eucalyptus oil.

Another embodiment comprises cyclosporin A, Tween 81®, and Eucalyptus oil.

Another embodiment comprises cyclosporin A, Tween 85®, and Eucalyptus oil.

Another embodiment comprises cyclosporin A, a Sucrose or Glucose Ester, or Derivative thereof, and Eucalyptus oil.

Another embodiment comprises cyclosporin A, DeSulf GOS-P-60WCG®, and Eucalyptus oil.

Another embodiment comprises cyclosporin A, Glucam E-20 Distearate®, and Eucalyptus oil.

Another embodiment comprises cyclosporin A, Glucamate DOE-120®, and Eucalyptus oil.

Another embodiment comprises cyclosporin A, Glucamate SSE-20®, and Eucalyptus oil.

Another embodiment comprises cyclosporin A, Glucate DO®, and Eucalyptus oil.

Another embodiment comprises cyclosporin A, Glucate SS®, and Eucalyptus oil.

Another embodiment comprises cyclosporin A, Glucopon 425 UP®, and Eucalyptus oil.

Another embodiment comprises cyclosporin A, Isolan IS®, and Eucalyptus oil.

Another embodiment comprises cyclosporin A, Mazon 40®, and Eucalyptus oil.

Another embodiment comprises cyclosporin A, Montanov 82®, and Eucalyptus oil.

Another embodiment comprises cyclosporin A, Montanov 202®, and Eucalyptus oil.

Another embodiment comprises cyclosporin A, Montanov S®, and Eucalyptus oil.

Another embodiment comprises cyclosporin A, Rheozan®, and Eucalyptus oil.

Another embodiment comprises cyclosporin A, Simulsol AS 48®, and Eucalyptus oil.

Another embodiment comprises cyclosporin A, Simulsol SL 4®, and Eucalyptus oil.

Another embodiment comprises cyclosporin A, Simulsol SL 10®, and Eucalyptus oil.

Another embodiment comprises cyclosporin A, Simulsol SL 11W®, and Eucalyptus oil.

Another embodiment comprises cyclosporin A, Simulsol SL 55®, and Eucalyptus oil.

Another embodiment comprises cyclosporin A, Suga Nate 100 and 160®, and Eucalyptus oil.

Another embodiment comprises cyclosporin A, Tego Care 450®, and Eucalyptus oil.

Another embodiment comprises cyclosporin A, Tego Care CG 90®, and Eucalyptus oil.

Another embodiment comprises cyclosporin A, Tego Care PS®, and Eucalyptus oil.

Another embodiment comprises cyclosporin A, Tegosoft PSE 141 G®, and Eucalyptus oil.

Another embodiment comprises cyclosporin A, Tegotens G 826®, and Eucalyptus oil.

Another embodiment comprises cyclosporin A, Triton BG-10 (70%)®, and Eucalyptus oil.

Another embodiment comprises cyclosporin A, Triton CG-110 (60%)®, and Eucalyptus oil.

Another embodiment comprises cyclosporin A, Wickenol 545®, and Eucalyptus oil.

Another embodiment comprises cyclosporin A, an alcohol, and Sesame oil.

Another embodiment comprises cyclosporin A, Diglycerol®, and Sesame oil.

Another embodiment comprises cyclosporin A, Hetoxide GT-80®, and Sesame oil.

Another embodiment comprises cyclosporin A, Lexemul BEO®, and Sesame oil.

Another embodiment comprises cyclosporin A, Polyglycerol-3®, and Sesame oil.

Another embodiment comprises cyclosporin A, Redicote E Series®, and Sesame oil.

Another embodiment comprises cyclosporin A, Simulsol OX 1005L®, and Sesame oil.

Another embodiment comprises cyclosporin A, Stanfax 567®, and Sesame oil.

Another embodiment comprises cyclosporin A, TA-1618®, and Sesame oil.

Another embodiment comprises cyclosporin A, Witconol H-31A®, and Sesame oil.

Another embodiment comprises cyclosporin A, an amine oxide, and Sesame oil.

Another embodiment comprises cyclosporin A, AO-405®, and Sesame oil.

Another embodiment comprises cyclosporin A, AO-455®, and Sesame oil.

Another embodiment comprises cyclosporin A, AO 728 Special®, and Sesame oil.

Another embodiment comprises cyclosporin A, Barlox 12®, and Sesame oil.

Another embodiment comprises cyclosporin A, Barlox 14®, and Sesame oil.

Another embodiment comprises cyclosporin A, Burcoxide Lo®, and Sesame oil.

Another embodiment comprises cyclosporin A, Caloxamine LO®, and Sesame oil.

Another embodiment comprises cyclosporin A, Chemoxide CAW®, and Sesame oil.

Another embodiment comprises cyclosporin A, Chemoxide LM-30®, and Sesame oil.

Another embodiment comprises cyclosporin A, Chemoxide LO®, and Sesame oil.

Another embodiment comprises cyclosporin A, Chemoxide MO®, and Sesame oil.

Another embodiment comprises cyclosporin A, Colalux CAO-35®, and Sesame oil.

Another embodiment comprises cyclosporin A, Colalux LO®, and Sesame oil.

Another embodiment comprises cyclosporin A, DeMox CAP®, and Sesame oil.

Another embodiment comprises cyclosporin A, DeMox CSG-30®, and Sesame oil.

Another embodiment comprises cyclosporin A, DeMox LAO®, and Sesame oil.

Another embodiment comprises cyclosporin A, Emcol L®, and Sesame oil.

Another embodiment comprises cyclosporin A, Empigen OB®, and Sesame oil.

Another embodiment comprises cyclosporin A, Empigen OS/A®, and Sesame oil.

Another embodiment comprises cyclosporin A, Foamox CDO®, and Sesame oil.

Another embodiment comprises cyclosporin A, Foamox DMM®, and Sesame oil.

Another embodiment comprises cyclosporin A, Foamox DMS®, and Sesame oil.

Another embodiment comprises cyclosporin A, Genaminox KC®, and Sesame oil.

Another embodiment comprises cyclosporin A, Genaminox LA®, and Sesame oil.

Another embodiment comprises cyclosporin A, Hartofoam SAO®, and Sesame oil.

Another embodiment comprises cyclosporin A, Hartox DMCD®, and Sesame oil.

Another embodiment comprises cyclosporin A, Lipowax DAT®, and Sesame oil.

Another embodiment comprises cyclosporin A, Lipowax PB Pastilles®, and Sesame oil.

Another embodiment comprises cyclosporin A, Mackamine C8®, and Sesame oil.

Another embodiment comprises cyclosporin A, Mackamine C10®, and Sesame oil.

Another embodiment comprises cyclosporin A, Mackamine C14®, and Sesame oil.

Another embodiment comprises cyclosporin A, Mackamine CAO®, and Sesame oil.

Another embodiment comprises cyclosporin A, Mackamine CO®, and Sesame oil.

Another embodiment comprises cyclosporin A, Mackamine LO®, and Sesame oil.

Another embodiment comprises cyclosporin A, Mackamine O2®, and Sesame oil.

Another embodiment comprises cyclosporin A, Mackamine SAO®, and Sesame oil.

Another embodiment comprises cyclosporin A, Mackamine SO®, and Sesame oil.

Another embodiment comprises cyclosporin A, Mazox KCAO®, and Sesame oil.

Another embodiment comprises cyclosporin A, Monalac MO®, and Sesame oil.

Another embodiment comprises cyclosporin A, Norfox LDA®, and Sesame oil.

Another embodiment comprises cyclosporin A, Rhodamox LO®, and Sesame oil.

Another embodiment comprises cyclosporin A, Schercamox C-AA®, and Sesame oil.

Another embodiment comprises cyclosporin A, Schercamox DMA®, and Sesame oil.

Another embodiment comprises cyclosporin A, Schercamox DML®, and Sesame oil.

Another embodiment comprises cyclosporin A, Schercamox DMM®, and Sesame oil.

Another embodiment comprises cyclosporin A, Schercamox DMS®, and Sesame oil.

Another embodiment comprises cyclosporin A, Tegotens DO®, and Sesame oil.

Another embodiment comprises cyclosporin A, Tomah AO-14-2®, and Sesame oil.

Another embodiment comprises cyclosporin A, Triaminox CDO®, and Sesame oil.

Another embodiment comprises cyclosporin A, a block polymer, and Sesame oil.

Another embodiment comprises cyclosporin A, AL 2070®, and Sesame oil.

Another embodiment comprises cyclosporin A, Antarox 17-R-2®, and Sesame oil.

Another embodiment comprises cyclosporin A, Antarox 25-R-2®, and Sesame oil.

Another embodiment comprises cyclosporin A, Antarox 31-R-1®, and Sesame oil.

Another embodiment comprises cyclosporin A, Antarox P-84®, and Sesame oil.

Another embodiment comprises cyclosporin A, Antarox P-104/H®, and Sesame oil.

Another embodiment comprises cyclosporin A, Arnox BP-Series®, and Sesame oil.

Another embodiment comprises cyclosporin A, Chemonic 435®, and Sesame oil.

Another embodiment comprises cyclosporin A, Chemonic D-25®, and Sesame oil.

Another embodiment comprises cyclosporin A, Chemonic PL Series®, and Sesame oil.

Another embodiment comprises cyclosporin A, Ethox L-121®, and Sesame oil.

Another embodiment comprises cyclosporin A, Ethox L-122®, and Sesame oil.

Another embodiment comprises cyclosporin A, Genapol PF-10®, and Sesame oil.

Another embodiment comprises cyclosporin A, Genapol PF-20®, and Sesame oil.

Another embodiment comprises cyclosporin A, Genapol PF-40A®, and Sesame oil.

Another embodiment comprises cyclosporin A, Norfox 2-LF®, and Sesame oil.

Another embodiment comprises cyclosporin A, Simulsol NW 342®, and Sesame oil.

Another embodiment comprises cyclosporin A, T-Det BP-1®, and Sesame oil.

Another embodiment comprises cyclosporin A, T-Det XD®, and Sesame oil.

Another embodiment comprises cyclosporin A, T-Det XH®, and Sesame oil.

Another embodiment comprises cyclosporin A, Triton CF-32®, and Sesame oil.

Another embodiment comprises cyclosporin A, Witconol 171®, and Sesame oil.

Another embodiment comprises cyclosporin A, Witconol 324®, and Sesame oil.

Another embodiment comprises cyclosporin A, Witconol 324D®, and Sesame oil.

Another embodiment comprises cyclosporin A, Witconol PD-2000®, and Sesame oil.

Another embodiment comprises cyclosporin A, a Carboxylated Alcohol or Alkylphenol Ethoxylate, and Sesame oil.

Another embodiment comprises cyclosporin A, Emcol CN-6®, and Sesame oil.

Another embodiment comprises cyclosporin A, Ethcarb®, and Sesame oil.

Another embodiment comprises cyclosporin A, Gemtex WNT-Conc®, and Sesame oil.

Another embodiment comprises cyclosporin A, Incrodet TD7-C®, and Sesame oil.

Another embodiment comprises cyclosporin A, Marlinat CM 105/80®, and Sesame oil.

Another embodiment comprises cyclosporin A, Marlowet 1072®, and Sesame oil.

Another embodiment comprises cyclosporin A, Marlowet 4530®, and Sesame oil.

Another embodiment comprises cyclosporin A, Marlowet 4530 LF®, and Sesame oil.

Another embodiment comprises cyclosporin A, Marlowet 4534®, and Sesame oil.

Another embodiment comprises cyclosporin A, Marlowet 4538®, and Sesame oil.

Another embodiment comprises cyclosporin A, Marlowet 4539®, and Sesame oil.

Another embodiment comprises cyclosporin A, Marlowet 4539 LF®, and Sesame oil.

Another embodiment comprises cyclosporin A, Marlowet 4541®, and Sesame oil.

Another embodiment comprises cyclosporin A, Miranate LEC-80®, and Sesame oil.

Another embodiment comprises cyclosporin A, Sandopan B®, and Sesame oil.

Another embodiment comprises cyclosporin A, Sandopan B Modified®, and Sesame oil.

Another embodiment comprises cyclosporin A, Sandopan LS-24 Gel®, and Sesame oil.

Another embodiment comprises cyclosporin A, Surfine T-A®, and Sesame oil.

Another embodiment comprises cyclosporin A, a Carboxylic Acid/Fatty Acid, and Sesame oil.

Another embodiment comprises cyclosporin A, Colaterge RAM®, and Sesame oil.

Another embodiment comprises cyclosporin A, Colatrope INC®, and Sesame oil.

Another embodiment comprises cyclosporin A, Crodacid B®, and Sesame oil.

Another embodiment comprises cyclosporin A, DeTrope CA-100®, and Sesame oil.

Another embodiment comprises cyclosporin A, Latol MTO®, and Sesame oil.

Another embodiment comprises cyclosporin A, Lumulse CC-33 K®, and Sesame oil.

Another embodiment comprises cyclosporin A, Mulls 2218®, and Sesame oil.

Another embodiment comprises cyclosporin A, OL-600®, and Sesame oil.

Another embodiment comprises cyclosporin A, OL-800®, and Sesame oil.

Another embodiment comprises cyclosporin A, R-910®, and Sesame oil.

Another embodiment comprises cyclosporin A, S-210®, and Sesame oil.

Another embodiment comprises cyclosporin A, Sandopan DTC Acid®, and Sesame oil.

Another embodiment comprises cyclosporin A, Sandopan LS 24 N®, and Sesame oil.

Another embodiment comprises cyclosporin A, Sandopan MA-18®, and Sesame oil.

Another embodiment comprises cyclosporin A, an Ethoxylated Alcohol, and Sesame oil.

Another embodiment comprises cyclosporin A, Adsee 799®, and Sesame oil.

Another embodiment comprises cyclosporin A, Adsee 799®, and Sesame oil.

Another embodiment comprises cyclosporin A, Alfonic 610-3.5®, and Sesame oil.

Another embodiment comprises cyclosporin A, Alfonic 810-2®, and Sesame oil.

Another embodiment comprises cyclosporin A, Alfonic 810-6®, and Sesame oil.

Another embodiment comprises cyclosporin A, Alfonic 1012-3®, and Sesame oil.

Another embodiment comprises cyclosporin A, Atfonic 1012-5®, and Sesame oil.

Another embodiment comprises cyclosporin A, Alfonic 1216CO-1.5®, and Sesame oil.

Another embodiment comprises cyclosporin A, Alfonic 1216CO-7®, and Sesame oil.

Another embodiment comprises cyclosporin A, Alfonic 1412-3®, and Sesame oil.

Another embodiment comprises cyclosporin A, Alfonic 1412-7®, and Sesame oil.

Another embodiment comprises cyclosporin A, Arlasolve 200®, and Sesame oil.

Another embodiment comprises cyclosporin A, Arlasolve 200 Liquid®, and Sesame oil.

Another embodiment comprises cyclosporin A, Armix 180-C®, and Sesame oil.

Another embodiment comprises cyclosporin A, Armix 183®, and Sesame oil.

Another embodiment comprises cyclosporin A, Armul 2404®, and Sesame oil.

Another embodiment comprises cyclosporin A, Armul 2404®, and Sesame oil.

Another embodiment comprises cyclosporin A, Atlas EMJ-C®, and Sesame oil.

Another embodiment comprises cyclosporin A, Atlas G-2109®, and Sesame oil.

Another embodiment comprises cyclosporin A, Atlas G-3886®, and Sesame oil.

Another embodiment comprises cyclosporin A, Atlas G-3890®, and Sesame oil.

Another embodiment comprises cyclosporin A, Bio Soft E-200®, and Sesame oil.

Another embodiment comprises cyclosporin A, Bio Soft E-300®, and Sesame oil.

Another embodiment comprises cyclosporin A, Bio Soft E-400®, and Sesame oil.

Another embodiment comprises cyclosporin A, Bio Soft EN 600®, and Sesame oil.

Another embodiment comprises cyclosporin A, Bio Soft TD-400®, and Sesame oil.

Another embodiment comprises cyclosporin A, Bio Soft TD-630®, and Sesame oil.

Another embodiment comprises cyclosporin A, Brij 30®, and Sesame oil.

Another embodiment comprises cyclosporin A, Brij 52®, and Sesame oil.

Another embodiment comprises cyclosporin A, Brij 56®, and Sesame oil.

Another embodiment comprises cyclosporin A, Brij 58®, and Sesame oil.

Another embodiment comprises cyclosporin A, Brij 72®, and Sesame oil.

Another embodiment comprises cyclosporin A, Brij 76®, and Sesame oil.

Another embodiment comprises cyclosporin A, Brij 78®, and Sesame oil.

Another embodiment comprises cyclosporin A, Brij 93®, and Sesame oil.

Another embodiment comprises cyclosporin A, Brij 97®, and Sesame oil.

Another embodiment comprises cyclosporin A, Brij 98®, and Sesame oil.

Another embodiment comprises cyclosporin A, Brij 700®, and Sesame oil.

Another embodiment comprises cyclosporin A, Brij 700 S®, and Sesame oil.

Another embodiment comprises cyclosporin A, Brij 721®, and Sesame oil.

Another embodiment comprises cyclosporin A, Brij 721 S®, and Sesame oil.

Another embodiment comprises cyclosporin A, Burcoterge CDG®, and Sesame oil.

Another embodiment comprises cyclosporin A, Canasol AT 600®, and Sesame oil.

Another embodiment comprises cyclosporin A, Canasol AT 800®, and Sesame oil.

Another embodiment comprises cyclosporin A, Canasol AT 1200®, and Sesame oil.

Another embodiment comprises cyclosporin A, Canasol BJ 35®, and Sesame oil.

Another embodiment comprises cyclosporin A, Canasol BJ 36®, and Sesame oil.

Another embodiment comprises cyclosporin A, Canasol BJ 52®, and Sesame oil.

Another embodiment comprises cyclosporin A, Canasol BJ 58®, and Sesame oil.

Another embodiment comprises cyclosporin A, Canasol BJ 72®, and Sesame oil.

Another embodiment comprises cyclosporin A, Canasol BJ 78®, and Sesame oil.

Another embodiment comprises cyclosporin A, Canasol BJ 98®, and Sesame oil.

Another embodiment comprises cyclosporin A, Canasol BJ 307®, and Sesame oil.

Another embodiment comprises cyclosporin A, Cerfak 1400®, and Sesame oil.

Another embodiment comprises cyclosporin A, Cetomacrogol 1000 BP®, and Sesame oil.

Another embodiment comprises cyclosporin A, Chemonic C-2®, and Sesame oil.

Another embodiment comprises cyclosporin A, Chemonic C-10®, and Sesame oil.

Another embodiment comprises cyclosporin A, Chemonic C-20®, and Sesame oil.

Another embodiment comprises cyclosporin A, Chemonic CT-12®, and Sesame oil.

Another embodiment comprises cyclosporin A, Chemonic CT-20®, and Sesame oil.

Another embodiment comprises cyclosporin A, Chemonic CT-30®, and Sesame oil.

Another embodiment comprises cyclosporin A, Chemonic CT-55®, and Sesame oil.

Another embodiment comprises cyclosporin A, Chemonic G-7®, and Sesame oil.

Another embodiment comprises cyclosporin A, Chemonic G-26®, and Sesame oil.

Another embodiment comprises cyclosporin A, Chemonic L-4®, and Sesame oil.

Another embodiment comprises cyclosporin A, Chemonic L-7®, and Sesame oil.

Another embodiment comprises cyclosporin A, Chemonic L-12®, and Sesame oil.

Another embodiment comprises cyclosporin A, Chemonic L-23®, and Sesame oil.

Another embodiment comprises cyclosporin A, Chemonic O-2®, and Sesame oil.

Another embodiment comprises cyclosporin A, Chemonic O-5®, and Sesame oil.

Another embodiment comprises cyclosporin A, Chemonic O-10®, and Sesame oil.

Another embodiment comprises cyclosporin A, Chemonic O-20®, and Sesame oil.

Another embodiment comprises cyclosporin A, Chemonic S-2®, and Sesame oil.

Another embodiment comprises cyclosporin A, Chemonic S-10®, and Sesame oil.

Another embodiment comprises cyclosporin A, Chemonic S-20®, and Sesame oil.

Another embodiment comprises cyclosporin A, Colamulse FE®, and Sesame oil.

Another embodiment comprises cyclosporin A, Cremophor A 20®, and Sesame oil.

Another embodiment comprises cyclosporin A, Cremophor SA 2®, and Sesame oil.

Another embodiment comprises cyclosporin A, Dehydol 100®, and Sesame oil.

Another embodiment comprises cyclosporin A, Dehydol O-4®, and Sesame oil.

Another embodiment comprises cyclosporin A, Delonic C-18®, and Sesame oil.

Another embodiment comprises cyclosporin A, DeSonic 6T®, and Sesame oil.

Another embodiment comprises cyclosporin A, DeSonic 9D®, and Sesame oil.

Another embodiment comprises cyclosporin A, DeSonic 9T®, and Sesame oil.

Another embodiment comprises cyclosporin A, DeSonic 12D®, and Sesame oil.

Another embodiment comprises cyclosporin A, DeSonic 12T®, and Sesame oil.

Another embodiment comprises cyclosporin A, DeSonic 15T®, and Sesame oil.

Another embodiment comprises cyclosporin A, DeSonic TDA-9®, and Sesame oil.

Another embodiment comprises cyclosporin A, DeThox GLG-7®, and Sesame oil.

Another embodiment comprises cyclosporin A, DeThox GLG-26®, and Sesame oil.

Another embodiment comprises cyclosporin A, DeThox LA-4®, and Sesame oil.

Another embodiment comprises cyclosporin A, DeThox LA-23®, and Sesame oil.

Another embodiment comprises cyclosporin A, DeThox SA-80®, and Sesame oil.

Another embodiment comprises cyclosporin A, Disponil O5®, and Sesame oil.

Another embodiment comprises cyclosporin A, Eccoterge EO-41B®, and Sesame oil.

Another embodiment comprises cyclosporin A, Empilan KA2.5/90®, and Sesame oil.

Another embodiment comprises cyclosporin A, Empilan KA5/90®, and Sesame oil.

Another embodiment comprises cyclosporin A, Empilan KM-20®, and Sesame oil.

Another embodiment comprises cyclosporin A, Empilan KM-50®, and Sesame oil.

Another embodiment comprises cyclosporin A, Empilan L-23®, and Sesame oil.

Another embodiment comprises cyclosporin A, Ethylan 25-3®, and Sesame oil.

Another embodiment comprises cyclosporin A, Ethylan 1204®, and Sesame oil.

Another embodiment comprises cyclosporin A, Ethylan DA-4®, and Sesame oil.

Another embodiment comprises cyclosporin A, Ethylan LA-230®, and Sesame oil.

Another embodiment comprises cyclosporin A, Ethylan SN®, and Sesame oil.

Another embodiment comprises cyclosporin A, Ethylan TD-60®, and Sesame oil.

Another embodiment comprises cyclosporin A, Ethylan TD-100®, and Sesame oil.

Another embodiment comprises cyclosporin A, Ethylan TD-1407®, and Sesame oil.

Another embodiment comprises cyclosporin A, Eumulgin B1®, and Sesame oil.

Another embodiment comprises cyclosporin A, Eumulgin B2®, and Sesame oil.

Another embodiment comprises cyclosporin A, Eumulgin B3®, and Sesame oil.

Another embodiment comprises cyclosporin A, Eumulgin O-10®, and Sesame oil.

Another embodiment comprises cyclosporin A, Flo Mo 80/20®, and Sesame oil.

Another embodiment comprises cyclosporin A, Flo Mo Low Foam®, and Sesame oil.

Another embodiment comprises cyclosporin A, Forlan C-24®, and Sesame oil.

Another embodiment comprises cyclosporin A, Genapol 1454®, and Sesame oil.

Another embodiment comprises cyclosporin A, Genapol BA-020®, and Sesame oil.

Another embodiment comprises cyclosporin A, Genapol BA-040®, and Sesame oil.

Another embodiment comprises cyclosporin A, Genapol C-100®, and Sesame oil.

Another embodiment comprises cyclosporin A, Genapol DA 060®, and Sesame oil.

Another embodiment comprises cyclosporin A, Genapol HS 020®, and Sesame oil.

Another embodiment comprises cyclosporin A, Genapol HS 200®, and Sesame oil.

Another embodiment comprises cyclosporin A, Genapol ID-040®, and Sesame oil.

Another embodiment comprises cyclosporin A, Genapol ID-060®, and Sesame oil.

Another embodiment comprises cyclosporin A, Genapol ID-090®, and Sesame oil.

Another embodiment comprises cyclosporin A, Genapol LA 010®, and Sesame oil.

Another embodiment comprises cyclosporin A, Genapol LA 020®, and Sesame oil.

Another embodiment comprises cyclosporin A, Genapol LA 030®, and Sesame oil.

Another embodiment comprises cyclosporin A, Genapol LA 040®, and Sesame oil.

Another embodiment comprises cyclosporin A, Genapol LA 050®, and Sesame oil.

Another embodiment comprises cyclosporin A, Genapol LA 060®, and Sesame oil.

Another embodiment comprises cyclosporin A, Genapol LA 070®, and Sesame oil.

Another embodiment comprises cyclosporin A, Genapol LA 070S®, and Sesame oil.

Another embodiment comprises cyclosporin A, Genapol LA 230®, and Sesame oil.

Another embodiment comprises cyclosporin A, Genapol O020®, and Sesame oil.

Another embodiment comprises cyclosporin A, Genapol O050®, and Sesame oil.

Another embodiment comprises cyclosporin A, Genapol O100®, and Sesame oil.

Another embodiment comprises cyclosporin A, Genapol O200®, and Sesame oil.

Another embodiment comprises cyclosporin A, Genapol SA 030®, and Sesame oil.

Another embodiment comprises cyclosporin A, Genapol SA 120®, and Sesame oil.

Another embodiment comprises cyclosporin A, Genapol T-020®, and Sesame oil.

Another embodiment comprises cyclosporin A, Genapol UD-030®, and Sesame oil.

Another embodiment comprises cyclosporin A, Genapol UD-050®, and Sesame oil.

Another embodiment comprises cyclosporin A, Genapol UD-070®, and Sesame oil.

Another embodiment comprises cyclosporin A, Genapol UD-079®, and Sesame oil.

Another embodiment comprises cyclosporin A, Genapol UD-080®, and Sesame oil.

Another embodiment comprises cyclosporin A, Genapol UD-110®, and Sesame oil.

Another embodiment comprises cyclosporin A, Genapol X 030®, and Sesame oil.

Another embodiment comprises cyclosporin A, Genapol X 050®, and Sesame oil.

Another embodiment comprises cyclosporin A, Genapol X 060®, and Sesame oil.

Another embodiment comprises cyclosporin A, Genapol X 070®, and Sesame oil.

Another embodiment comprises cyclosporin A, Genapol X 080®, and Sesame oil.

Another embodiment comprises cyclosporin A, Genapol X 100®, and Sesame oil.

Another embodiment comprises cyclosporin A, Genapol X159®, and Sesame oil.

Another embodiment comprises cyclosporin A, Generol 122 E5®, and Sesame oil.

Another embodiment comprises cyclosporin A, Generol 122 E25®, and Sesame oil.

Another embodiment comprises cyclosporin A, Hostacerin T-3®, and Sesame oil.

Another embodiment comprises cyclosporin A, Iconol LF 110®, and Sesame oil.

Another embodiment comprises cyclosporin A, Incropol CS-20®, and Sesame oil.

Another embodiment comprises cyclosporin A, Lexemul CS-20®, and Sesame oil.

Another embodiment comprises cyclosporin A, Liponic EG-1®, and Sesame oil.

Another embodiment comprises cyclosporin A, Lipowax D®, and Sesame oil.

Another embodiment comprises cyclosporin A, Lipowax G®, and Sesame oil.

Another embodiment comprises cyclosporin A, Lipowax NI®, and Sesame oil.

Another embodiment comprises cyclosporin A, Lipowax P®, and Sesame oil.

Another embodiment comprises cyclosporin A, Lipowax P-31®, and Sesame oil.

Another embodiment comprises cyclosporin A, Lipowax PR®, and Sesame oil.

Another embodiment comprises cyclosporin A, Lumulse CS-20®, and Sesame oil.

Another embodiment comprises cyclosporin A, Macol CSA-20®, and Sesame oil.

Another embodiment comprises cyclosporin A, Marlox B 24/50®, and Sesame oil.

Another embodiment comprises cyclosporin A, Mazawet 77®, and Sesame oil.

Another embodiment comprises cyclosporin A, Norfox 1713®, and Sesame oil.

Another embodiment comprises cyclosporin A, Norfox 2579®, and Sesame oil.

Another embodiment comprises cyclosporin A, Nordox Lo Foam®, and Sesame oil.

Another embodiment comprises cyclosporin A, Promulgen D®, and Sesame oil.

Another embodiment comprises cyclosporin A, Promulgen G®, and Sesame oil.

Another embodiment comprises cyclosporin A, Renex 30®, and Sesame oil.

Another embodiment comprises cyclosporin A, Renex 36®, and Sesame oil.

Another embodiment comprises cyclosporin A, Rhodasurf A 24®, and Sesame oil.

Another embodiment comprises cyclosporin A, Rhodasurf AAE-10®, and Sesame oil.

Another embodiment comprises cyclosporin A, Rhodasurf BEH-25®, and Sesame oil.

Another embodiment comprises cyclosporin A, Rhodasurf BEH-40®, and Sesame oil.

Another embodiment comprises cyclosporin A, Rhodasurf DA 530®, and Sesame oil.

Another embodiment comprises cyclosporin A, Rhodasurf DA 630®, and Sesame oil.

Another embodiment comprises cyclosporin A, Rhodasurf DA 639®, and Sesame oil.

Another embodiment comprises cyclosporin A, Rhodasurf LAN-23®, and Sesame oil.

Another embodiment comprises cyclosporin A, Rhodasurf ON-870®, and Sesame oil.

Another embodiment comprises cyclosporin A, Rhodasurf ON-877®, and Sesame oil.

Another embodiment comprises cyclosporin A, Rhodasurf TB-970 FLK®, and Sesame oil.

Another embodiment comprises cyclosporin A, Ritacet-20®, and Sesame oil.

Another embodiment comprises cyclosporin A, Ritachol 1000®, and Sesame oil.

Another embodiment comprises cyclosporin A, Ritachol 2000®, and Sesame oil.

Another embodiment comprises cyclosporin A, Ritachol 5000®, and Sesame oil.

Another embodiment comprises cyclosporin A, Ritox 35®, and Sesame oil.

Another embodiment comprises cyclosporin A, Surfonic DA-4®, and Sesame oil.

Another embodiment comprises cyclosporin A, Surfonic DA-6®, and Sesame oil.

Another embodiment comprises cyclosporin A, Surfonic L46-7®, and Sesame oil.

Another embodiment comprises cyclosporin A, Surfonic POA®, and Sesame oil.

Another embodiment comprises cyclosporin A, Synthrapol KB®, and Sesame oil.

Another embodiment comprises cyclosporin A, Teginacid®, and Sesame oil.

Another embodiment comprises cyclosporin A, Teginacid C®, and Sesame oil.

Another embodiment comprises cyclosporin A, Tegotens EC 11®, and Sesame oil.

Another embodiment comprises cyclosporin A, Tinegal NA®, and Sesame oil.

Another embodiment comprises cyclosporin A, Tomadol 400®, and Sesame oil.

Another embodiment comprises cyclosporin A, Tomadol 600®, and Sesame oil.

Another embodiment comprises cyclosporin A, Tomadol 900®, and Sesame oil.

Another embodiment comprises cyclosporin A, Uniperol O®, and Sesame oil.

Another embodiment comprises cyclosporin A, Witconol SN Series®, and Sesame oil.

Another embodiment comprises cyclosporin A, an Ethoxylated Alkylphenol, and Sesame oil.

Another embodiment comprises cyclosporin A, Antarox LF-222®, and Sesame oil.

Another embodiment comprises cyclosporin A, Atlox 775®, and Sesame oil.

Another embodiment comprises cyclosporin A, Caloxylate N-9®, and Sesame oil.

Another embodiment comprises cyclosporin A, Canasol NF-1000®, and Sesame oil.

Another embodiment comprises cyclosporin A, Canasol NF-3000®, and Sesame oil.

Another embodiment comprises cyclosporin A, Canasol NF-3070®, and Sesame oil.

Another embodiment comprises cyclosporin A, Canasol OF 1670®, and Sesame oil.

Another embodiment comprises cyclosporin A, Canasol OF 2570®, and Sesame oil.

Another embodiment comprises cyclosporin A, Canasol OF 4070®, and Sesame oil.

Another embodiment comprises cyclosporin A, Chemax DNP-8®, and Sesame oil.

Another embodiment comprises cyclosporin A, Chemax DNP-18®, and Sesame oil.

Another embodiment comprises cyclosporin A, Chemax DNP-150/50®, and Sesame oil.

Another embodiment comprises cyclosporin A, DeSonic 1.5N®, and Sesame oil.

Another embodiment comprises cyclosporin A, DeSonic 4N®, and Sesame oil.

Another embodiment comprises cyclosporin A, DeSonic 5N®, and Sesame oil.

Another embodiment comprises cyclosporin A, DeSonic 6D®, and Sesame oil.

Another embodiment comprises cyclosporin A, DeSonic 6N®, and Sesame oil.

Another embodiment comprises cyclosporin A, DeSonic 7N®, and Sesame oil.

Another embodiment comprises cyclosporin A, DeSonic 9N®, and Sesame oil.

Another embodiment comprises cyclosporin A, DeSonic 10D®, and Sesame oil.

Another embodiment comprises cyclosporin A, DeSonic 11N®, and Sesame oil.

Another embodiment comprises cyclosporin A, DeSonic 12N®, and Sesame oil.

Another embodiment comprises cyclosporin A, DeSonic 15N®, and Sesame oil.

Another embodiment comprises cyclosporin A, DeSonic 20N®, and Sesame oil.

Another embodiment comprises cyclosporin A, Eccoscour RC®, and Sesame oil.

Another embodiment comprises cyclosporin A, Eccoterge EO-100®, and Sesame oil.

Another embodiment comprises cyclosporin A, Emulsifier 632/90%®, and Sesame oil.

Another embodiment comprises cyclosporin A, Geronol AG-821®, and Sesame oil.

Another embodiment comprises cyclosporin A, Gradonic N-95®, and Sesame oil.

Another embodiment comprises cyclosporin A, Hetoxide NP-4®, and Sesame oil.

Another embodiment comprises cyclosporin A, Hetoxide NP-30®, and Sesame oil.

Another embodiment comprises cyclosporin A, Hostapal N-100®, and Sesame oil.

Another embodiment comprises cyclosporin A, Hostapal N-110®, and Sesame oil.

Another embodiment comprises cyclosporin A, Igepal CTA-639W®, and Sesame oil.

Another embodiment comprises cyclosporin A, Igepal DAP-9®, and Sesame oil.

Another embodiment comprises cyclosporin A, Igepal OD-410®, and Sesame oil.

Another embodiment comprises cyclosporin A, Igepal SS-837®, and Sesame oil.

Another embodiment comprises cyclosporin A, Lipocol NP-9 USP®, and Sesame oil.

Another embodiment comprises cyclosporin A, Macol DNP-10®, and Sesame oil.

Another embodiment comprises cyclosporin A, Marlophen NP 5®, and Sesame oil.

Another embodiment comprises cyclosporin A, Marlophen P 1®, and Sesame oil.

Another embodiment comprises cyclosporin A, Surfonic NB®, and Sesame oil.

Another embodiment comprises cyclosporin A, Surfonic OPB-307®, and Sesame oil.

Another embodiment comprises cyclosporin A, Surfonic OPB-407®, and Sesame oil.

Another embodiment comprises cyclosporin A, Syn Fac 334®, and Sesame oil.

Another embodiment comprises cyclosporin A, Syn Fac 8216®, and Sesame oil.

Another embodiment comprises cyclosporin A, Triton N-57®, and Sesame oil.

Another embodiment comprises cyclosporin A, Trycol 6956®, and Sesame oil.

Another embodiment comprises cyclosporin A, Trycol 6961®, and Sesame oil.

Another embodiment comprises cyclosporin A, Trycol 6964®, and Sesame oil.

Another embodiment comprises cyclosporin A, Trycol 6969®, and Sesame oil.

Another embodiment comprises cyclosporin A, Trycol 6974®, and Sesame oil.

Another embodiment comprises cyclosporin A, Witbreak DRB-127®, and Sesame oil.

Another embodiment comprises cyclosporin A, Witbreak DRB-127®, and Sesame oil.

Another embodiment comprises cyclosporin A, Witconol NP Series®, and Sesame oil.

Another embodiment comprises cyclosporin A, an Ethoxylated Aryl Phenol, and Sesame oil.

Another embodiment comprises cyclosporin A, Soprophor BSU®, and Sesame oil.

Another embodiment comprises cyclosporin A, Soprophor CY/8®, and Sesame oil.

Another embodiment comprises cyclosporin A, Soprophor S/25®, and Sesame oil.

Another embodiment comprises cyclosporin A, Witconol NIO®, and Sesame oil.

Another embodiment comprises cyclosporin A, Witconol NIW®, and Sesame oil.

Another embodiment comprises cyclosporin A, Witconol S-100®, and Sesame oil.

Another embodiment comprises cyclosporin A, an Ethoxylated Fatty Acid, and Sesame oil.

Another embodiment comprises cyclosporin A, Aldo PGHMS®, and Sesame oil.

Another embodiment comprises cyclosporin A, Alkamuls TO-15/HR®, and Sesame oil.

Another embodiment comprises cyclosporin A, Armotan AL-69-66®, and Sesame oil.

Another embodiment comprises cyclosporin A, Cerasynt 840®, and Sesame oil.

Another embodiment comprises cyclosporin A, Cerasynt 945®, and Sesame oil.

Another embodiment comprises cyclosporin A, Crystal Inhibitor No. 5®, and Sesame oil.

Another embodiment comprises cyclosporin A, DeThox Acid L-9®, and Sesame oil.

Another embodiment comprises cyclosporin A, DeThox Acid S-8®, and Sesame oil.

Another embodiment comprises cyclosporin A, Ethofat 242/25®, and Sesame oil.

Another embodiment comprises cyclosporin A, Hydropalat 65®, and Sesame oil.

Another embodiment comprises cyclosporin A, Lipo EGMS®, and Sesame oil.

Another embodiment comprises cyclosporin A, Lipopeg 2 DL®, and Sesame oil.

Another embodiment comprises cyclosporin A, Lipopeg 4 DL®, and Sesame oil.

Another embodiment comprises cyclosporin A, Lipopeg 4-L®, and Sesame oil.

Another embodiment comprises cyclosporin A, Lipopeg 39-S®, and Sesame oil.

Another embodiment comprises cyclosporin A, Lipopeg 4-S®, and Sesame oil.

Another embodiment comprises cyclosporin A, Lipopeg 10-S®, and Sesame oil.

Another embodiment comprises cyclosporin A, Lipopeg 100-S®, and Sesame oil.

Another embodiment comprises cyclosporin A, Lipopeg 6000 DS®, and Sesame oil.

Another embodiment comprises cyclosporin A, Lumulse 40-L®, and Sesame oil.

Another embodiment comprises cyclosporin A, Lumulse 40-S®, and Sesame oil.

Another embodiment comprises cyclosporin A, Lumulse 42-L®, and Sesame oil.

Another embodiment comprises cyclosporin A, Lumulse 42-S®, and Sesame oil.

Another embodiment comprises cyclosporin A, Lumulse 100-S®, and Sesame oil.

Another embodiment comprises cyclosporin A, Lumulse 602-S®, and Sesame oil.

Another embodiment comprises cyclosporin A, Magrabar PGE-20-0®, and Sesame oil.

Another embodiment comprises cyclosporin A, Magrabar PGE-20L®, and Sesame oil.

Another embodiment comprises cyclosporin A, Magrabar PGE-20T®, and Sesame oil.

Another embodiment comprises cyclosporin A, Magrabar PGE-22-0®, and Sesame oil.

Another embodiment comprises cyclosporin A, Magrabar PGE-22L®, and Sesame oil.

Another embodiment comprises cyclosporin A, Magrabar PGE-22T®, and Sesame oil.

Another embodiment comprises cyclosporin A, Magrabar PGE-40-0®, and Sesame oil.

Another embodiment comprises cyclosporin A, Magrabar PGE-40L®, and Sesame oil.

Another embodiment comprises cyclosporin A, Magrabar PGE-40T®, and Sesame oil.

Another embodiment comprises cyclosporin A, Magrabar PGE-42-0®, and Sesame oil.

Another embodiment comprises cyclosporin A, Magrabar PGE-42L®, and Sesame oil.

Another embodiment comprises cyclosporin A, Magrabar PGE-42T®, and Sesame oil.

Another embodiment comprises cyclosporin A, Magrabar PGE-60-0®, and Sesame oil.

Another embodiment comprises cyclosporin A, Magrabar PGE-60L®, and Sesame oil.

Another embodiment comprises cyclosporin A, Magrabar PGE-60T®, and Sesame oil.

Another embodiment comprises cyclosporin A, Magrabar PGE-62-0®, and Sesame oil.

Another embodiment comprises cyclosporin A, Magrabar PGE-62L®, and Sesame oil.

Another embodiment comprises cyclosporin A, Magrabar PGE-62T®, and Sesame oil.

Another embodiment comprises cyclosporin A, Mapeg S-40K®, and Sesame oil.

Another embodiment comprises cyclosporin A, Marlowet OTS®, and Sesame oil.

Another embodiment comprises cyclosporin A, Naturechem PGR®, and Sesame oil.

Another embodiment comprises cyclosporin A, PG No. 4®, and Sesame oil.

Another embodiment comprises cyclosporin A, Renex 20®, and Sesame oil.

Another embodiment comprises cyclosporin A, Ritox 52®, and Sesame oil.

Another embodiment comprises cyclosporin A, Ritox 53®, and Sesame oil.

Another embodiment comprises cyclosporin A, Ritox 59®, and Sesame oil.

Another embodiment comprises cyclosporin A, Surfax 8916/A®, and Sesame oil.

Another embodiment comprises cyclosporin A, Tego Acid S 40 P®, and Sesame oil.

Another embodiment comprises cyclosporin A, Tego Acid S 100 P®, and Sesame oil.

Another embodiment comprises cyclosporin A, Tween 20®, and Sesame oil.

Another embodiment comprises cyclosporin A, Volpo 131®, and Sesame oil.

Another embodiment comprises cyclosporin A, an Ethoxylated Fatty Ester or Oil, and Sesame oil.

Another embodiment comprises cyclosporin A, Acconon 6-C10®, and Sesame oil.

Another embodiment comprises cyclosporin A, Acconbn CC-6®, and Sesame oil.

Another embodiment comprises cyclosporin A, Acconon CO-7®, and Sesame oil.

Another embodiment comprises cyclosporin A, Aldosperse 40/60 FG®, and Sesame oil.

Another embodiment comprises cyclosporin A, Aldosperse ML-23®, and Sesame oil.

Another embodiment comprises cyclosporin A, Aldosperse MS-20 FG®, and Sesame oil.

Another embodiment comprises cyclosporin A, Alkamuls EL-620®, and Sesame oil.

Another embodiment comprises cyclosporin A, Alkamuls EL-719®, and Sesame oil.

Another embodiment comprises cyclosporin A, Alkamuls EL-985®, and Sesame oil.

Another embodiment comprises cyclosporin A, Arlatone G®, and Sesame oil.

Another embodiment comprises cyclosporin A, Arlatone T®, and Sesame oil.

Another embodiment comprises cyclosporin A, Atlas G-1045A®, and Sesame oil.

Another embodiment comprises cyclosporin A, Atlas G-1086®, and Sesame oil.

Another embodiment comprises cyclosporin A, Atlas G-1087®, and Sesame oil.

Another embodiment comprises cyclosporin A, Atlas G-1089®, and Sesame oil.

Another embodiment comprises cyclosporin A, Atlas G-1096®, and Sesame oil.

Another embodiment comprises cyclosporin A, Atlas G-1292®, and Sesame oil.

Another embodiment comprises cyclosporin A, Atlas G-1293®, and Sesame oil.

Another embodiment comprises cyclosporin A, Atlas G-1300®, and Sesame oil.

Another embodiment comprises cyclosporin A, Atlas G-7076®, and Sesame oil.

Another embodiment comprises cyclosporin A, Capmul EMG®, and Sesame oil.

Another embodiment comprises cyclosporin A, Chemonic CO-40®, and Sesame oil.

Another embodiment comprises cyclosporin A, Chemonic LI-3®, and Sesame oil.

Another embodiment comprises cyclosporin A, Chemonic LI-7®, and Sesame oil.

Another embodiment comprises cyclosporin A, Cirrasol GM®, and Sesame oil.

Another embodiment comprises cyclosporin A, Cremophor CO 40®, and Sesame oil.

Another embodiment comprises cyclosporin A, Cremophor CO 410®, and Sesame oil.

Another embodiment comprises cyclosporin A, Cremophor EL®, and Sesame oil.

Another embodiment comprises cyclosporin A, Cremophor GC7®, and Sesame oil.

Another embodiment comprises cyclosporin A, Cremophor RH-40®, and Sesame oil.

Another embodiment comprises cyclosporin A, Crovol A-40®, and Sesame oil.

Another embodiment comprises cyclosporin A, Crovol A-70®, and Sesame oil.

Another embodiment comprises cyclosporin A, Crovol M-70®, and Sesame oil.

Another embodiment comprises cyclosporin A, Crovol PK-70®, and Sesame oil.

Another embodiment comprises cyclosporin A, Cutina E-24®, and Sesame oil.

Another embodiment comprises cyclosporin A, Dacospin 12-R®, and Sesame oil.

Another embodiment comprises cyclosporin A, Dehymuls HRE-7®, and Sesame oil.

Another embodiment comprises cyclosporin A, DeSonic 30C®, and Sesame oil.

Another embodiment comprises cyclosporin A, DeSonic 36C®, and Sesame oil.

Another embodiment comprises cyclosporin A, DeSonic 40C®, and Sesame oil.

Another embodiment comprises cyclosporin A, Durfax 60®, and Sesame oil.

Another embodiment comprises cyclosporin A, Durfax 65®, and Sesame oil.

Another embodiment comprises cyclosporin A, Durfax 80®, and Sesame oil.

Another embodiment comprises cyclosporin A, Durfax EOM®, and Sesame oil.

Another embodiment comprises cyclosporin A, Eccoterge NF-2®, and Sesame oil.

Another embodiment comprises cyclosporin A, Emulpon CO-360®, and Sesame oil.

Another embodiment comprises cyclosporin A, Emulpon CO-550®, and Sesame oil.

Another embodiment comprises cyclosporin A, Emulsogen EL®, and Sesame oil.

Another embodiment comprises cyclosporin A, Emulsogen HCO 040®, and Sesame oil.

Another embodiment comprises cyclosporin A, Emulsogen HCO 060®, and Sesame oil.

Another embodiment comprises cyclosporin A, Emulsynt 1055®, and Sesame oil.

Another embodiment comprises cyclosporin A, Ethox 3095®, and Sesame oil.

Another embodiment comprises cyclosporin A, Eumulgin RO-40®, and Sesame oil.

Another embodiment comprises cyclosporin A, Genapol G-260®, and Sesame oil.

Another embodiment comprises cyclosporin A, Glycosperse L-20®, and Sesame oil.

Another embodiment comprises cyclosporin A, Glycosperse O-5®, and Sesame oil.

Another embodiment comprises cyclosporin A, Glycosperse O-20®, and Sesame oil.

Another embodiment comprises cyclosporin A, Glycosperse O-20 FG®, and Sesame oil.

Another embodiment comprises cyclosporin A, Glycosperse S-20®, and Sesame oil.

Another embodiment comprises cyclosporin A, Glycosperse S-20 FG®, and Sesame oil.

Another embodiment comprises cyclosporin A, Glycosperse TS-20®, and Sesame oil.

Another embodiment comprises cyclosporin A, Glycosperse TS-20 FG®, and Sesame oil.

Another embodiment comprises cyclosporin A, Hetan SL®, and Sesame oil.

Another embodiment comprises cyclosporin A, Hetan SO®, and Sesame oil.

Another embodiment comprises cyclosporin A, Hetan SS®, and Sesame oil.

Another embodiment comprises cyclosporin A, Hetoxide C-2®, and Sesame oil.

Another embodiment comprises cyclosporin A, Hetoxide C-9®, and Sesame oil.

Another embodiment comprises cyclosporin A, Hetoxide C-15®, and Sesame oil.

Another embodiment comprises cyclosporin A, Hetoxide C-25®, and Sesame oil.

Another embodiment comprises cyclosporin A, Hetoxide C-40®, and Sesame oil.

Another embodiment comprises cyclosporin A, Hetoxide C-200®, and Sesame oil.

Another embodiment comprises cyclosporin A, Hetoxide C-200-50%®, and Sesame oil.

Another embodiment comprises cyclosporin A, Hetoxide GC-30®, and Sesame oil.

Another embodiment comprises cyclosporin A, Hetoxide HC-60®, and Sesame oil.

Another embodiment comprises cyclosporin A, Ice No. 2®, and Sesame oil.

Another embodiment comprises cyclosporin A, Incrocas 30/40®, and Sesame oil.

Another embodiment comprises cyclosporin A, Lexol EC®, and Sesame oil.

Another embodiment comprises cyclosporin A, Lexol EO®, and Sesame oil.

Another embodiment comprises cyclosporin A, Lipocol HCO-40®, and Sesame oil.

Another embodiment comprises cyclosporin A, Lipocol HCO-60®, and Sesame oil.

Another embodiment comprises cyclosporin A, Lipocol O-3 Special®, and Sesame oil.

Another embodiment comprises cyclosporin A, Lipopeg 2-L®, and Sesame oil.

Another embodiment comprises cyclosporin A, Lipopeg 4-DO®, and Sesame oil.

Another embodiment comprises cyclosporin A, Lipopeg 4-DS®, and Sesame oil.

Another embodiment comprises cyclosporin A, Lipovol GTB®, and Sesame oil.

Another embodiment comprises cyclosporin A, Lonzest SML-20®, and Sesame oil.

Another embodiment comprises cyclosporin A, Lonzest SMO-20®, and Sesame oil.

Another embodiment comprises cyclosporin A, Lonzest SMS-20®, and Sesame oil.

Another embodiment comprises cyclosporin A, Lonzest STO-20®, and Sesame oil.

Another embodiment comprises cyclosporin A, Lonzest STS-20®, and Sesame oil.

Another embodiment comprises cyclosporin A, Lumulse GR-40®, and Sesame oil.

Another embodiment comprises cyclosporin A, Lumulse GRH-40®, and Sesame oil.

Another embodiment comprises cyclosporin A, Lumulse POE (7) GML®, and Sesame oil.

Another embodiment comprises cyclosporin A, Lumulse POE (12) Glyc®, and Sesame oil.

Another embodiment comprises cyclosporin A, Lumulse POE (40) MS KP®, and Sesame oil.

Another embodiment comprises cyclosporin A, Marlowet 4750®, and Sesame oil.

Another embodiment comprises cyclosporin A, Marlowet LVS®, and Sesame oil.

Another embodiment comprises cyclosporin A, Marlowet R 11®, and Sesame oil.

Another embodiment comprises cyclosporin A, Marlowet R 40®, and Sesame oil.

Another embodiment comprises cyclosporin A, Mazol 80 MGK®, and Sesame oil.

Another embodiment comprises cyclosporin A, Nonionic Emulsifier T-9®, and Sesame oil.

Another embodiment comprises cyclosporin A, Oronal LCG®, and Sesame oil.

Another embodiment comprises cyclosporin A, Polyderm PPI-CO-200®, and Sesame oil.

Another embodiment comprises cyclosporin A, Polyderm PPI-CO-40®, and Sesame oil.

Another embodiment comprises cyclosporin A, Rewoderm LI 520-70®, and Sesame oil.

Another embodiment comprises cyclosporin A, Ritapeg 150 DS®, and Sesame oil.

Another embodiment comprises cyclosporin A, Softigen 767®, and Sesame oil.

Another embodiment comprises cyclosporin A, Surfactol 318®, and Sesame oil.

Another embodiment comprises cyclosporin A, Surfactol 365®, and Sesame oil.

Another embodiment comprises cyclosporin A, Syn Lube 107®, and Sesame oil.

Another embodiment comprises cyclosporin A, Syn Lube 728®, and Sesame oil.

Another embodiment comprises cyclosporin A, Syn Lube 1632H®, and Sesame oil.

Another embodiment comprises cyclosporin A, Syn Lube 6277-A®, and Sesame oil.

Another embodiment comprises cyclosporin A, T-Det C-20®, and Sesame oil.

Another embodiment comprises cyclosporin A, T-Det C-40®, and Sesame oil.

Another embodiment comprises cyclosporin A, Tally 100 Plus®, and Sesame oil.

Another embodiment comprises cyclosporin A, Uniperol EL®, and Sesame oil.

Another embodiment comprises cyclosporin A, a Fatty Ester, and Sesame oil.

Another embodiment comprises cyclosporin A, Actralube-Syn 147®, and Sesame oil.

Another embodiment comprises cyclosporin A, Atlas G-1556®, and Sesame oil.

Another embodiment comprises cyclosporin A, Atlas G-1564®, and Sesame oil.

Another embodiment comprises cyclosporin A, Atlasol Base Oil S®, and Sesame oil.

Another embodiment comprises cyclosporin A, Base ML®, and Sesame oil.

Another embodiment comprises cyclosporin A, Base MT®, and Sesame oil.

Another embodiment comprises cyclosporin A, Cerasynt 303®, and Sesame oil.

Another embodiment comprises cyclosporin A, Dermol 1012®, and Sesame oil.

Another embodiment comprises cyclosporin A, Kemester 4000®, and Sesame oil.

Another embodiment comprises cyclosporin A, Lactipol S®, and Sesame oil.

Another embodiment comprises cyclosporin A, Magrabar PGO®, and Sesame oil.

Another embodiment comprises cyclosporin A, Mayco Base BF®, and Sesame oil.

Another embodiment comprises cyclosporin A, Methyl Linoleate®, and Sesame oil.

Another embodiment comprises cyclosporin A, Pationic 122A®, and Sesame oil.

Another embodiment comprises cyclosporin A, Pationic 138C®, and Sesame oil.

Another embodiment comprises cyclosporin A, Pationic CSL®, and Sesame oil.

Another embodiment comprises cyclosporin A, Pationic ISL®, and Sesame oil.

Another embodiment comprises cyclosporin A, Pationic SBL®, and Sesame oil.

Another embodiment comprises cyclosporin A, Pationic SSL®, and Sesame oil.

Another embodiment comprises cyclosporin A, Ritasol®, and Sesame oil.

Another embodiment comprises cyclosporin A, Tego Alkanol CS 20®, and Sesame oil.

Another embodiment comprises cyclosporin A, Tego Alkanol L23 P®, and Sesame oil.

Another embodiment comprises cyclosporin A, Tego Alkanol S2®, and Sesame oil.

Another embodiment comprises cyclosporin A, Tego Alkanol S20 P®, and Sesame oil.

Another embodiment comprises cyclosporin A, Triemulsifier 600 MS®, and Sesame oil.

Another embodiment comprises cyclosporin A, a Glycerol Esters, and Sesame oil.

Another embodiment comprises cyclosporin A, Agro #9 Wint SBO®, and Sesame oil.

Another embodiment comprises cyclosporin A, Ahcovel Base 700®, and Sesame oil.

Another embodiment comprises cyclosporin A, Aldo HMS FG®, and Sesame oil.

Another embodiment comprises cyclosporin A, Aldo MLD®, and Sesame oil.

Another embodiment comprises cyclosporin A, Aldo MLD FG®, and Sesame oil.

Another embodiment comprises cyclosporin A, Aldo MO FG®, and Sesame oil.

Another embodiment comprises cyclosporin A, Aldo MS®, and Sesame oil.

Another embodiment comprises cyclosporin A, Aldo MS FG®, and Sesame oil.

Another embodiment comprises cyclosporin A, Aldo MS LG FG®, and Sesame oil.

Another embodiment comprises cyclosporin A, Aldo MSD®, and Sesame oil.

Another embodiment comprises cyclosporin A, Aldo MSD FG®, and Sesame oil.

Another embodiment comprises cyclosporin A, Aldosperse O-20 FG®, and Sesame oil.

Another embodiment comprises cyclosporin A, Aldosperse TS-20 FG®, and Sesame oil.

Another embodiment comprises cyclosporin A, Aldosperse TS-40 FG®, and Sesame oil.

Another embodiment comprises cyclosporin A, Arlacel 165®, and Sesame oil.

Another embodiment comprises cyclosporin A, Arlacel 186®, and Sesame oil.

Another embodiment comprises cyclosporin A, Capmul GMO®, and Sesame oil.

Another embodiment comprises cyclosporin A, Capmul GMS®, and Sesame oil.

Another embodiment comprises cyclosporin A, Caprol 3GO®, and Sesame oil.

Another embodiment comprises cyclosporin A, Caprol 3GVS®, and Sesame oil.

Another embodiment comprises cyclosporin A, Caprol 6G2S®, and Sesame oil.

Another embodiment comprises cyclosporin A, Caprol 10G40®, and Sesame oil.

Another embodiment comprises cyclosporin A, Caprol 10G100®, and Sesame oil.

Another embodiment comprises cyclosporin A, Caprol ET®, and Sesame oil.

Another embodiment comprises cyclosporin A, Caprol PGE860®, and Sesame oil.

Another embodiment comprises cyclosporin A, Cerasynt 945®, and Sesame oil.

Another embodiment comprises cyclosporin A, Cerasynt GMS®, and Sesame oil.

Another embodiment comprises cyclosporin A, Cerasynt Q®, and Sesame oil.

Another embodiment comprises cyclosporin A, Cerasynt SD®, and Sesame oil.

Another embodiment comprises cyclosporin A, Cerasynt WM®, and Sesame oil.

Another embodiment comprises cyclosporin A, Chemsperse 14®, and Sesame oil.

Another embodiment comprises cyclosporin A, Cremophor GO-32®, and Sesame oil.

Another embodiment comprises cyclosporin A, Cremophor GS11®, and Sesame oil.

Another embodiment comprises cyclosporin A, Cremophor GS-32®, and Sesame oil.

Another embodiment comprises cyclosporin A, Cutina KD-16®, and Sesame oil.

Another embodiment comprises cyclosporin A, Dehymuls PGPH®, and Sesame oil.

Another embodiment comprises cyclosporin A, Dermol DGDIS®, and Sesame oil.

Another embodiment comprises cyclosporin A, Dermol DGMIS®, and Sesame oil.

Another embodiment comprises cyclosporin A, Dermol G-76®, and Sesame oil.

Another embodiment comprises cyclosporin A, Dermol G-7DI®, and Sesame oil.

Another embodiment comprises cyclosporin A, Dermol NGDI®, and Sesame oil.

Another embodiment comprises cyclosporin A, Dermolan GLH®, and Sesame oil.

Another embodiment comprises cyclosporin A, Drewmulse GMO®, and Sesame oil.

Another embodiment comprises cyclosporin A, Drewpol 3-5-M®, and Sesame oil.

Another embodiment comprises cyclosporin A, Durlac 100 W®, and Sesame oil.

Another embodiment comprises cyclosporin A, Dur-Lo®, and Sesame oil.

Another embodiment comprises cyclosporin A, Dynasan 118®, and Sesame oil.

Another embodiment comprises cyclosporin A, EC-25®, and Sesame oil.

Another embodiment comprises cyclosporin A, EM 40®, and Sesame oil.

Another embodiment comprises cyclosporin A, Emerest 2400®, and Sesame oil.

Another embodiment comprises cyclosporin A, Emerest 2452®, and Sesame oil.

Another embodiment comprises cyclosporin A, Empilan G-26®, and Sesame oil.

Another embodiment comprises cyclosporin A, Genapol TSM®, and Sesame oil.

Another embodiment comprises cyclosporin A, Hostacerin DGI®, and Sesame oil.

Another embodiment comprises cyclosporin A, Hostacerin DGL®, and Sesame oil.

Another embodiment comprises cyclosporin A, Hostacerin DGMS®, and Sesame oil.

Another embodiment comprises cyclosporin A, Hostacerin DGSB®, and Sesame oil.

Another embodiment comprises cyclosporin A, Ice No. 2®, and Sesame oil.

Another embodiment comprises cyclosporin A, Imwitor 742®, and Sesame oil.

Another embodiment comprises cyclosporin A, Imwitor 780 K®, and Sesame oil.

Another embodiment comprises cyclosporin A, Imwitor 960 Flakes®, and Sesame oil.

Another embodiment comprises cyclosporin A, Isolan GI 34®, and Sesame oil.

Another embodiment comprises cyclosporin A, Isolan GO 33®, and Sesame oil.

Another embodiment comprises cyclosporin A, Kemester 1000®, and Sesame oil.

Another embodiment comprises cyclosporin A, Kemester 2000®, and Sesame oil.

Another embodiment comprises cyclosporin A, Kemester 2000®, and Sesame oil.

Another embodiment comprises cyclosporin A, Kemester 6000SE®, and Sesame oil.

Another embodiment comprises cyclosporin A, Lamecreme DGE 18®, and Sesame oil.

Another embodiment comprises cyclosporin A, Lexemul 515®, and Sesame oil.

Another embodiment comprises cyclosporin A, Lexemul 561®, and Sesame oil.

Another embodiment comprises cyclosporin A, Lexemul AR®, and Sesame oil.

Another embodiment comprises cyclosporin A, Lexemul AS®, and Sesame oil.

Another embodiment comprises cyclosporin A, Lexemul GDL®, and Sesame oil.

Another embodiment comprises cyclosporin A, Lexemul T®, and Sesame oil.

Another embodiment comprises cyclosporin A, Lipomulse 165®, and Sesame oil.

Another embodiment comprises cyclosporin A, Lumulse GML K®, and Sesame oil.

Another embodiment comprises cyclosporin A, Lumulse GMO K®, and Sesame oil.

Another embodiment comprises cyclosporin A, Lumulse GMR K®, and Sesame oil.

Another embodiment comprises cyclosporin A, Lumulse GMT K®, and Sesame oil.

Another embodiment comprises cyclosporin A, Magrabar GMC®, and Sesame oil.

Another embodiment comprises cyclosporin A, Magrabar GMO-CK®, and Sesame oil.

Another embodiment comprises cyclosporin A, Magrabar MDG-5050®, and Sesame oil.

Another embodiment comprises cyclosporin A, Magrabar PGO-315®, and Sesame oil.

Another embodiment comprises cyclosporin A, Magrabar PGO-1010®, and Sesame oil.

Another embodiment comprises cyclosporin A, Mazol 300K®, and Sesame oil.

Another embodiment comprises cyclosporin A, Mazol GMO-K®, and Sesame oil.

Another embodiment comprises cyclosporin A, Mazol GMS-K®, and Sesame oil.

Another embodiment comprises cyclosporin A, Mazol PG031-K®, and Sesame oil.

Another embodiment comprises cyclosporin A, Miglyol 812®, and Sesame oil.

Another embodiment comprises cyclosporin A, Norfox 165C®, and Sesame oil.

Another embodiment comprises cyclosporin A, Schercemol GMIS®, and Sesame oil.

Another embodiment comprises cyclosporin A, Tegin®, and Sesame oil.

Another embodiment comprises cyclosporin A, Tegin 4100 Pellets®, and Sesame oil.

Another embodiment comprises cyclosporin A, Tegin M Pellets®, and Sesame oil.

Another embodiment comprises cyclosporin A, Tegin OV®, and Sesame oil.

Another embodiment comprises cyclosporin A, Teginacid H®, and Sesame oil.

Another embodiment comprises cyclosporin A, Tego Cosmo P813®, and Sesame oil.

Another embodiment comprises cyclosporin A, Wickenol 535®, and Sesame oil.

Another embodiment comprises cyclosporin A, Witconol 14®, and Sesame oil.

Another embodiment comprises cyclosporin A, Witconol 14®, and Sesame oil.

Another embodiment comprises cyclosporin A, Witconol 14F®, and Sesame oil.

Another embodiment comprises cyclosporin A, Witconol 18L®, and Sesame oil.

Another embodiment comprises cyclosporin A, Witconol GOT®, and Sesame oil.

Another embodiment comprises cyclosporin A, Witconol MST®, and Sesame oil.

Another embodiment comprises cyclosporin A, Witconol RHT®, and Sesame oil.

Another embodiment comprises cyclosporin A, a Glycol Ester, and Sesame oil.

Another embodiment comprises cyclosporin A, Alkamuls 600 DO®, and Sesame oil.

Another embodiment comprises cyclosporin A, Alkamuls SEG®, and Sesame oil.

Another embodiment comprises cyclosporin A, Atlas EM-2®, and Sesame oil.

Another embodiment comprises cyclosporin A, Cerasynt IP®, and Sesame oil.

Another embodiment comprises cyclosporin A, Cerasynt M®, and Sesame oil.

Another embodiment comprises cyclosporin A, Cerasynt MN®, and Sesame oil.

Another embodiment comprises cyclosporin A, Cerasynt PA®, and Sesame oil.

Another embodiment comprises cyclosporin A, Chemsperse EGDS®, and Sesame oil.

Another embodiment comprises cyclosporin A, Chemsperse EGMS®, and Sesame oil.

Another embodiment comprises cyclosporin A, Colonial Monolaurin®, and Sesame oil.

Another embodiment comprises cyclosporin A, DeMuls SGE-95®, and Sesame oil.

Another embodiment comprises cyclosporin A, Eccoterge 200®, and Sesame oil.

Another embodiment comprises cyclosporin A, Emerest 2380®, and Sesame oil.

Another embodiment comprises cyclosporin A, Ethox 2610®, and Sesame oil.

Another embodiment comprises cyclosporin A, Ethox DO-9®, and Sesame oil.

Another embodiment comprises cyclosporin A, Ethox DO-14®, and Sesame oil.

Another embodiment comprises cyclosporin A, Ethox SO-9®, and Sesame oil.

Another embodiment comprises cyclosporin A, Fizul MD-318®, and Sesame oil.

Another embodiment comprises cyclosporin A, Genapol EGDS-VHP®, and Sesame oil.

Another embodiment comprises cyclosporin A, Genapol TS Powder®, and Sesame oil.

Another embodiment comprises cyclosporin A, Hostacerin WO®, and Sesame oil.

Another embodiment comprises cyclosporin A, Inversol 140®, and Sesame oil.

Another embodiment comprises cyclosporin A, Kemester 104®, and Sesame oil.

Another embodiment comprises cyclosporin A, Kemester 205®, and Sesame oil.

Another embodiment comprises cyclosporin A, Kemester 226®, and Sesame oil.

Another embodiment comprises cyclosporin A, Kemester 5221SE®, and Sesame oil.

Another embodiment comprises cyclosporin A, Kemester EGDS®, and Sesame oil.

Another embodiment comprises cyclosporin A, Lexemul EGDS®, and Sesame oil.

Another embodiment comprises cyclosporin A, Lexemul EGMS®, and Sesame oil.

Another embodiment comprises cyclosporin A, Lexemul P®, and Sesame oil.

Another embodiment comprises cyclosporin A, Lipo DGLS®, and Sesame oil.

Another embodiment comprises cyclosporin A, Lipo EGDS®, and Sesame oil.

Another embodiment comprises cyclosporin A, Lipo PGMS®, and Sesame oil.

Another embodiment comprises cyclosporin A, Liposorb S-4®, and Sesame oil.

Another embodiment comprises cyclosporin A, Liposorb TO-20®, and Sesame oil.

Another embodiment comprises cyclosporin A, Lumulse PGO®, and Sesame oil.

Another embodiment comprises cyclosporin A, Mackester EGDS®, and Sesame oil.

Another embodiment comprises cyclosporin A, Mackester EGMS®, and Sesame oil.

Another embodiment comprises cyclosporin A, Mackester GSTP®, and Sesame oil.

Another embodiment comprises cyclosporin A, Mackester Series®, and Sesame oil.

Another embodiment comprises cyclosporin A, Magrabar PDG-50®, and Sesame oil.

Another embodiment comprises cyclosporin A, Mapeg 6000 DS®, and Sesame oil.

Another embodiment comprises cyclosporin A, Marlowet 4702®, and Sesame oil.

Another embodiment comprises cyclosporin A, Monalube 305®, and Sesame oil.

Another embodiment comprises cyclosporin A, Monalube 310®, and Sesame oil.

Another embodiment comprises cyclosporin A, Monalube 315®, and Sesame oil.

Another embodiment comprises cyclosporin A, Monalube 320®, and Sesame oil.

Another embodiment comprises cyclosporin A, Monalube 325®, and Sesame oil.

Another embodiment comprises cyclosporin A, Monalube 330®, and Sesame oil.

Another embodiment comprises cyclosporin A, Naturechem PGHS®, and Sesame oil.

Another embodiment comprises cyclosporin A, Polycastorol PLO-840®, and Sesame oil.

Another embodiment comprises cyclosporin A, Polytex 10M®, and Sesame oil.

Another embodiment comprises cyclosporin A, Ritasynt IP®, and Sesame oil.

Another embodiment comprises cyclosporin A, Ross Chem PEG 600 DT®, and Sesame oil.

Another embodiment comprises cyclosporin A, Schercemol PGMS®, and Sesame oil.

Another embodiment comprises cyclosporin A, Sponto H-44C®, and Sesame oil.

Another embodiment comprises cyclosporin A, Tegin G®, and Sesame oil.

Another embodiment comprises cyclosporin A, Witbreak DGE-182®, and Sesame oil.

Another embodiment comprises cyclosporin A, Witbreak DGE-182®, and Sesame oil.

Another embodiment comprises cyclosporin A, Witbreak DRA-21®, and Sesame oil.

Another embodiment comprises cyclosporin A, Witbreak DRA-21®, and Sesame oil.

Another embodiment comprises cyclosporin A, Witbreak DRA-50®, and Sesame oil.

Another embodiment comprises cyclosporin A, Witbreak DRA-50®, and Sesame oil.

Another embodiment comprises cyclosporin A, Witconol F26-46®, and Sesame oil.

Another embodiment comprises cyclosporin A, Witconol H-32®, and Sesame oil.

Another embodiment comprises cyclosporin A, Witconol H-33®, and Sesame oil.

Another embodiment comprises cyclosporin A, Witconol H-35A®, and Sesame oil.

Another embodiment comprises cyclosporin A, Witconol RHP®, and Sesame oil.

Another embodiment comprises cyclosporin A, a Lanolin-based Derivative, and Sesame oil.

Another embodiment comprises cyclosporin A, Amerchol CAB®, and Sesame oil.

Another embodiment comprises cyclosporin A, Amerchol L-101®, and Sesame oil.

Another embodiment comprises cyclosporin A, Amerlate LFA-LO®, and Sesame oil.

Another embodiment comprises cyclosporin A, Amerlate P®, and Sesame oil.

Another embodiment comprises cyclosporin A, Barre Common Degras®, and Sesame oil.

Another embodiment comprises cyclosporin A, Cholesterol NF®, and Sesame oil.

Another embodiment comprises cyclosporin A, Crodalan AWS®, and Sesame oil.

Another embodiment comprises cyclosporin A, Crodalan LA®, and Sesame oil.

Another embodiment comprises cyclosporin A, Emery 1650®, and Sesame oil.

Another embodiment comprises cyclosporin A, Emery 1650®, and Sesame oil.

Another embodiment comprises cyclosporin A, Emery 1740®, and Sesame oil.

Another embodiment comprises cyclosporin A, Emery 1740®, and Sesame oil.

Another embodiment comprises cyclosporin A, Forlan 500®, and Sesame oil.

Another embodiment comprises cyclosporin A, Forlan L®, and Sesame oil.

Another embodiment comprises cyclosporin A, Laneto 50®, and Sesame oil.

Another embodiment comprises cyclosporin A, Laneto 100®, and Sesame oil.

Another embodiment comprises cyclosporin A, Laneto AWS®, and Sesame oil.

Another embodiment comprises cyclosporin A, Lanfrax 1776®, and Sesame oil.

Another embodiment comprises cyclosporin A, Lanfrax 1776®, and Sesame oil.

Another embodiment comprises cyclosporin A, Lanogel 21®, and Sesame oil.

Another embodiment comprises cyclosporin A, Lipolan®, and Sesame oil.

Another embodiment comprises cyclosporin A, Lipolan 31®, and Sesame oil.

Another embodiment comprises cyclosporin A, OHlan®, and Sesame oil.

Another embodiment comprises cyclosporin A, Polychol 5®, and Sesame oil.

Another embodiment comprises cyclosporin A, Polychol 15®, and Sesame oil.

Another embodiment comprises cyclosporin A, Ritacetyl®, and Sesame oil.

Another embodiment comprises cyclosporin A, Ritachol®, and Sesame oil.

Another embodiment comprises cyclosporin A, Ritahydrox®, and Sesame oil.

Another embodiment comprises cyclosporin A, Ritalafa®, and Sesame oil.

Another embodiment comprises cyclosporin A, Ritalan®, and Sesame oil.

Another embodiment comprises cyclosporin A, Ritalan AWS®, and Sesame oil.

Another embodiment comprises cyclosporin A, Ritalan C®, and Sesame oil.

Another embodiment comprises cyclosporin A, Ritawax®, and Sesame oil.

Another embodiment comprises cyclosporin A, Ritawax AEO®, and Sesame oil.

Another embodiment comprises cyclosporin A, Ritawax ALA®, and Sesame oil.

Another embodiment comprises cyclosporin A, Solan/Solan 50/Super Solan®, and Sesame oil.

Another embodiment comprises cyclosporin A, Super Hartolan/Hartolan®, and Sesame oil.

Another embodiment comprises cyclosporin A, Supersat AWS-4®, and Sesame oil.

Another embodiment comprises cyclosporin A, Supersat AWS-24®, and Sesame oil.

Another embodiment comprises cyclosporin A, a Lecithin or Lecithin Derivative, and Sesame oil.

Another embodiment comprises cyclosporin A, Alcolec®, and Sesame oil.

Another embodiment comprises cyclosporin A, Lecithin®, and Sesame oil.

Another embodiment comprises cyclosporin A, Lexin K®, and Sesame oil.

Another embodiment comprises cyclosporin A, Natipide®, and Sesame oil.

Another embodiment comprises cyclosporin A, a Lignin or Lignin Derivative, and Sesame oil.

Another embodiment comprises cyclosporin A, Diwatex XP 9®, and Sesame oil.

Another embodiment comprises cyclosporin A, Dynasperse LCD®, and Sesame oil.

Another embodiment comprises cyclosporin A, Indulin SAL®, and Sesame oil.

Another embodiment comprises cyclosporin A, Indulin W-1®,and Sesame oil.

Another embodiment comprises cyclosporin A, Indulin W-5®, and Sesame oil.

Another embodiment comprises cyclosporin A, Lignosol FTA®, and Sesame oil.

Another embodiment comprises cyclosporin A, Lignosol SFX-65®, and Sesame oil.

Another embodiment comprises cyclosporin A, Marasperse 52 CP®, and Sesame oil.

Another embodiment comprises cyclosporin A, Marasperse AG®, and Sesame oil.

Another embodiment comprises cyclosporin A, Marasperse CBOS-4®,and Sesame oil.

Another embodiment comprises cyclosporin A, Ufoxane 2®, and Sesame oil.

Another embodiment comprises cyclosporin A, a Methyl Ester, and Sesame oil.

Another embodiment comprises cyclosporin A, E.B. Cleaner AK®, and Sesame oil.

Another embodiment comprises cyclosporin A, Oleocal ME-70®, and Sesame oil.

Another embodiment comprises cyclosporin A, Trade Name Company®, and Sesame oil.

Another embodiment comprises cyclosporin A, Methyl Esters (cont'd)®, and Sesame oil.

Another embodiment comprises cyclosporin A, Oleocal ME-92®, and Sesame oil.

Another embodiment comprises cyclosporin A, Oleocal ME-112®, and Sesame oil.

Another embodiment comprises cyclosporin A, Oleocal ME-130®, and Sesame oil.

Another embodiment comprises cyclosporin A, a Monoglyceride or a Derivative thereof, and Sesame oil.

Another embodiment comprises cyclosporin A, Dynacet 211®, and Sesame oil.

Another embodiment comprises cyclosporin A, Hetsorb S-20®, and Sesame oil.

Another embodiment comprises cyclosporin A, Imwitor 191®, and Sesame oil.

Another embodiment comprises cyclosporin A, Imwitor 370®, and Sesame oil.

Another embodiment comprises cyclosporin A, Imwitor 375®, and Sesame oil.

Another embodiment comprises cyclosporin A, Imwitor 900®, and Sesame oil.

Another embodiment comprises cyclosporin A, Imwitor 945®, and Sesame oil.

Another embodiment comprises cyclosporin A, Imwitor 2020®, and Sesame oil.

Another embodiment comprises cyclosporin A, Kemester 5500®, and Sesame oil.

Another embodiment comprises cyclosporin A, Kemester 6000®, and Sesame oil.

Another embodiment comprises cyclosporin A, Magrabar GMC®, and Sesame oil.

Another embodiment comprises cyclosporin A, Magrabar GMO-CK®, and Sesame oil.

Another embodiment comprises cyclosporin A, Magrabar GPC-10®, and Sesame oil.

Another embodiment comprises cyclosporin A, Magrabar MDG-5050®, and Sesame oil.

Another embodiment comprises cyclosporin A, Monalube 335®, and Sesame oil.

Another embodiment comprises cyclosporin A, Monoglycerides Glyceryl Monestearate Archer Daniels Midland Company®, and Sesame oil.

Another embodiment comprises cyclosporin A, Rita GMS®, and Sesame oil.

Another embodiment comprises cyclosporin A, Ritamulse SCG®, and Sesame oil.

Another embodiment comprises cyclosporin A, Softigen 701®, and Sesame oil.

Another embodiment comprises cyclosporin A, Tally 100 Plus®, and Sesame oil.

Another embodiment comprises cyclosporin A, a Polyethylene Glycol, and Sesame oil.

Another embodiment comprises cyclosporin A, Emulgade PL 68/50®, and Sesame oil.

Another embodiment comprises cyclosporin A, Lumulse PEG®, and Sesame oil.

Another embodiment comprises cyclosporin A, Rhodasurf PEG-400®, and Sesame oil.

Another embodiment comprises cyclosporin A, Rhodasurf PEG-600®, and Sesame oil.

Another embodiment comprises cyclosporin A, Witconol PEG-400®, and Sesame oil.

Another embodiment comprises cyclosporin A, a Polymeric Surfactant, and Sesame oil.

Another embodiment comprises cyclosporin A, Acritamer PNC-EG®, and Sesame oil.

Another embodiment comprises cyclosporin A, Ag-Rho DEP-775®, and Sesame oil.

Another embodiment comprises cyclosporin A, APG 325N Glycoside®, and Sesame oil.

Another embodiment comprises cyclosporin A, Aristoflex AVC®, and Sesame oil.

Another embodiment comprises cyclosporin A, Aristoflex HMB®, and Sesame oil.

Another embodiment comprises cyclosporin A, Burco NPS-225®, and Sesame oil.

Another embodiment comprises cyclosporin A, Burco NPS-816®, and Sesame oil.

Another embodiment comprises cyclosporin A, Chemccinate 5603®, and Sesame oil.

Another embodiment comprises cyclosporin A, Cosmedia Guar C-261N®, and Sesame oil.

Another embodiment comprises cyclosporin A, Gantrez S-95®, and Sesame oil.

Another embodiment comprises cyclosporin A, Glucopon 220 UP®, and Sesame oil.

Another embodiment comprises cyclosporin A, Glucopon 225 DK®, and Sesame oil.

Another embodiment comprises cyclosporin A, Glucopon 425 N®, and Sesame oil.

Another embodiment comprises cyclosporin A, Glucopon 600 UP®, and Sesame oil.

Another embodiment comprises cyclosporin A, Glucopon 625 UP®, and Sesame oil.

Another embodiment comprises cyclosporin A, Pemulen 1621®, and Sesame oil.

Another embodiment comprises cyclosporin A, Pemulen 1622®, and Sesame oil.

Another embodiment comprises cyclosporin A, Pemulen TR-1®, and Sesame oil.

Another embodiment comprises cyclosporin A, Pemulen TR-2®, and Sesame oil.

Another embodiment comprises cyclosporin A, Plantacare 818®, and Sesame oil.

Another embodiment comprises cyclosporin A, Plantapon LGC Sorb®, and Sesame oil.

Another embodiment comprises cyclosporin A, Plantaren 1200N®, and Sesame oil.

Another embodiment comprises cyclosporin A, Plantaren 2000N®, and Sesame oil.

Another embodiment comprises cyclosporin A, Viscolam AT 64®, and Sesame oil.

Another embodiment comprises cyclosporin A, Viscolam AT 64P®, and Sesame oil.

Another embodiment comprises cyclosporin A, Viscolam AT 100®, and Sesame oil.

Another embodiment comprises cyclosporin A, Viscolam MAC 7®, and Sesame oil.

Another embodiment comprises cyclosporin A, Viscolam SMC 20®, and Sesame oil.

Another embodiment comprises cyclosporin A, Witbreak RTC-323®, and Sesame oil.

Another embodiment comprises cyclosporin A, WSI 3700®, and Sesame oil.

Another embodiment comprises cyclosporin A, a Propoxylated or Ethoxylated Fatty Acid, Alcohol, or Alkyl Phenol, and Sesame oil.

Another embodiment comprises cyclosporin A, Antarox AA-60®, and Sesame oil.

Another embodiment comprises cyclosporin A, Antarox LF-224®, and Sesame oil.

Another embodiment comprises cyclosporin A, Burcomul DFE-45®, and Sesame oil.

Another embodiment comprises cyclosporin A, Burcoterge LFE-1000®, and Sesame oil.

Another embodiment comprises cyclosporin A, Chemal LF-25B®, and Sesame oil.

Another embodiment comprises cyclosporin A, Chemal LF-40B®, and Sesame oil.

Another embodiment comprises cyclosporin A, Dehypon LS-36®, and Sesame oil.

Another embodiment comprises cyclosporin A, Dehypon LS-36®, and Sesame oil.

Another embodiment comprises cyclosporin A, Dehypon LS-54®, and Sesame oil.

Another embodiment comprises cyclosporin A, Dehypon LS-54®, and Sesame oil.

Another embodiment comprises cyclosporin A, Delonic 100 VLF®, and Sesame oil.

Another embodiment comprises cyclosporin A, Delonic LF-60 MOD®, and Sesame oil.

Another embodiment comprises cyclosporin A, Empiderm B®, and Sesame oil.

Another embodiment comprises cyclosporin A, Ethylan 1206®, and Sesame oil.

Another embodiment comprises cyclosporin A, Ethylan NS-500K®, and Sesame oil.

Another embodiment comprises cyclosporin A, Ethylan NS-500LQ®, and Sesame oil.

Another embodiment comprises cyclosporin A, Genapol 1392®, and Sesame oil.

Another embodiment comprises cyclosporin A, Genapol 2317®, and Sesame oil.

Another embodiment comprises cyclosporin A, Genapol 26EP710®, and Sesame oil.

Another embodiment comprises cyclosporin A, Genapol EP 1022®, and Sesame oil.

Another embodiment comprises cyclosporin A, Genapol EP 1024®, and Sesame oil.

Another embodiment comprises cyclosporin A, Genapol EP 6068®, and Sesame oil.

Another embodiment comprises cyclosporin A, Genapol NP915®, and Sesame oil.

Another embodiment comprises cyclosporin A, Kieralon MFB®, and Sesame oil.,

Another embodiment comprises cyclosporin A, Lumisolve CSA-80 V®, and Sesame oil.

Another embodiment comprises cyclosporin A, Marlowet 5001®, and Sesame oil.

Another embodiment comprises cyclosporin A, Marlox FK 64®, and Sesame oil.

Another embodiment comprises cyclosporin A, Marlox MO 124®, and Sesame oil.

Another embodiment comprises cyclosporin A, Marlox S 58®, and Sesame oil.

Another embodiment comprises cyclosporin A, Nonatell 1003®, and Sesame oil.

Another embodiment comprises cyclosporin A, Nonatell 1038®, and Sesame oil.

Another embodiment comprises cyclosporin A, Nonatell 1052®, and Sesame oil.

Another embodiment comprises cyclosporin A, Nonatell 1061®, and Sesame oil.

Another embodiment comprises cyclosporin A, Nonatell 1075®, and Sesame oil.

Another embodiment comprises cyclosporin A, Nonatell 1088®, and Sesame oil.

Another embodiment comprises cyclosporin A, Nonatell 1123®, and Sesame oil.

Another embodiment comprises cyclosporin A, Nonatell 1153®, and Sesame oil.

Another embodiment comprises cyclosporin A, Nonatell 1161®, and Sesame oil.

Another embodiment comprises cyclosporin A, Nonatell 1172®, and Sesame oil.

Another embodiment comprises cyclosporin A, Nonatell 1181®, and Sesame oil.

Another embodiment comprises cyclosporin A, Norfox 36®, and Sesame oil.

Another embodiment comprises cyclosporin A, Procetyl AWS®, and Sesame oil.

Another embodiment comprises cyclosporin A, Sandoxylate SX 412®, and Sesame oil.

Another embodiment comprises cyclosporin A, Sandoxylate SX 418®, and Sesame oil.

Another embodiment comprises cyclosporin A, Surfonic JL-80X®, and Sesame oil.

Another embodiment comprises cyclosporin A, Surfonic JL-80X-B1®, and Sesame oil.

Another embodiment comprises cyclosporin A, Surfonic L4-29X®, and Sesame oil.

Another embodiment comprises cyclosporin A, Surfonic LF®, and Sesame oil.

Another embodiment comprises cyclosporin A, T-Det A826®, and Sesame oil.

Another embodiment comprises cyclosporin A, T-Det LF-416®, and Sesame oil.

Another embodiment comprises cyclosporin A, Tergitol Min-Foam 1X®, and Sesame oil.

Another embodiment comprises cyclosporin A, Tergitol Min-Foam 2X®, and Sesame oil.

Another embodiment comprises cyclosporin A, Triton CF-21®, and Sesame oil.

Another embodiment comprises cyclosporin A, Triton CF-76®, and Sesame oil.

Another embodiment comprises cyclosporin A, Triton XL-80N®, and Sesame oil.

Another embodiment comprises cyclosporin A, Witconol NS-98®, and Sesame oil.

Another embodiment comprises cyclosporin A, Witconol NS-108LQ®, and Sesame oil.

Another embodiment comprises cyclosporin A, Witconol NS-145®, and Sesame oil.

Another embodiment comprises cyclosporin A, Witconol NS-179®, and Sesame oil.

Another embodiment comprises cyclosporin A, a Protein-based Surfactant, and Sesame oil.

Another embodiment comprises cyclosporin A, AminoFoam W®, and Sesame oil.

Another embodiment comprises cyclosporin A, Amiter LGOD-2®, and Sesame oil.

Another embodiment comprises cyclosporin A, Amiter LGS-2®, and Sesame oil.

Another embodiment comprises cyclosporin A, Amiter LGS-5®, and Sesame oil.

Another embodiment comprises cyclosporin A, Lamepon S®, and Sesame oil.

Another embodiment comprises cyclosporin A, Lamepon S®, and Sesame oil.

Another embodiment comprises cyclosporin A, Maypon 4C®, and Sesame oil.

Another embodiment comprises cyclosporin A, May-Tein C®, and Sesame oil.

Another embodiment comprises cyclosporin A, May-Tein CT®, and Sesame oil.

Another embodiment comprises cyclosporin A, May-Tein KTS®, and Sesame oil.

Another embodiment comprises cyclosporin A, May-Tein SY®, and Sesame oil.

Another embodiment comprises cyclosporin A, Plantapon S®, and Sesame oil.

Another embodiment comprises cyclosporin A, Proteol APL®, and Sesame oil.

Another embodiment comprises cyclosporin A, Proteol OAT®, and Sesame oil.

Another embodiment comprises cyclosporin A, Pyroter CPI-40®, and Sesame oil.

Another embodiment comprises cyclosporin A, Pyroter GPI-25®, and Sesame oil.

Another embodiment comprises cyclosporin A, Supro-Tein S®, and Sesame oil.

Another embodiment comprises cyclosporin A, Supro-Tein V®, and Sesame oil.

Another embodiment comprises cyclosporin A,® and Sesame oil.

Another embodiment comprises cyclosporin A, a Sarcosine Derivative®, and Sesame oil.

Another embodiment comprises cyclosporin A, Crodasinic LS-30®, and Sesame oil.

Another embodiment comprises cyclosporin A, Vanseal CS®, and Sesame oil.

Another embodiment comprises cyclosporin A, Vanseal LS®, and Sesame oil.

Another embodiment comprises cyclosporin A, Vanseal MS®, and Sesame oil.

Another embodiment comprises cyclosporin A, Vanseal NACS-30®, and Sesame oil.

Another embodiment comprises cyclosporin A, Vanseal NALS-95®, and Sesame oil.

Another embodiment comprises cyclosporin A, Vanseal OS®, and Sesame oil.

Another embodiment comprises cyclosporin A,® and Sesame oil.

Another embodiment comprises cyclosporin A, a Silicone-based Surfactant®, and Sesame oil.

Another embodiment comprises cyclosporin A, Abil-B-9950®, and Sesame oil.

Another embodiment comprises cyclosporin A, Abil Care 85®, and Sesame oil.

Another embodiment comprises cyclosporin A, Abil EM 90®, and Sesame oil.

Another embodiment comprises cyclosporin A, Abil EM 97®, and Sesame oil.

Another embodiment comprises cyclosporin A, Abil WE-09®, and Sesame oil.

Another embodiment comprises cyclosporin A, Dow Corning 1248 Fluid®, and Sesame oil.

Another embodiment comprises cyclosporin A, Dow Corning 3225C®, and Sesame oil.

Another embodiment comprises cyclosporin A, Dow Corning 5200®, and Sesame oil.

Another embodiment comprises cyclosporin A, Dow Corning Q4-3667®, and Sesame oil.

Another embodiment comprises cyclosporin A, Monasil PCA®, and Sesame oil.

Another embodiment comprises cyclosporin A, Monasil PDM®, and Sesame oil.

Another embodiment comprises cyclosporin A, Monasil PLN®, and Sesame oil.

Another embodiment comprises cyclosporin A, Polyderm PPI-SI-WS®, and Sesame oil.

Another embodiment comprises cyclosporin A, Troysol 380W®, and Sesame oil.

Another embodiment comprises cyclosporin A, Troysol S366®, and Sesame oil.

Another embodiment comprises cyclosporin A, a Sorbitan Derivative, and Sesame oil.

Another embodiment comprises cyclosporin A, Alkamuls SML®, and Sesame oil.

Another embodiment comprises cyclosporin A, Alkamuls SMO®, and Sesame oil.

Another embodiment comprises cyclosporin A, Alkamuls STO®, and Sesame oil.

Another embodiment comprises cyclosporin A, Arlacel 20®, and Sesame oil.

Another embodiment comprises cyclosporin A, Arlacel 40®, and Sesame oil.

Another embodiment comprises cyclosporin A, Arlacel 60®, and Sesame oil.

Another embodiment comprises cyclosporin A, Arlacel 80®, and Sesame oil.

Another embodiment comprises cyclosporin A, Arlacel C®, and Sesame oil.

Another embodiment comprises cyclosporin A, Armul 21®, and Sesame oil.

Another embodiment comprises cyclosporin A, Atlox 80®, and Sesame oil.

Another embodiment comprises cyclosporin A, Atlox 847®, and Sesame oil.

Another embodiment comprises cyclosporin A, Atlox 1045A®, and Sesame oil.

Another embodiment comprises cyclosporin A, Canarcel 20®, and Sesame oil.

Another embodiment comprises cyclosporin A, Canarcel 60®, and Sesame oil.

Another embodiment comprises cyclosporin A, Canarcel 80®, and Sesame oil.

Another embodiment comprises cyclosporin A, Canarcel TW 20®, and Sesame oil.

Another embodiment comprises cyclosporin A, Canarcel TW 60®, and Sesame oil.

Another embodiment comprises cyclosporin A, Canarcel TW 80®, and Sesame oil.

Another embodiment comprises cyclosporin A, Coladet BSB-P®, and Sesame oil.

Another embodiment comprises cyclosporin A, Customulse O-20®, and Sesame oil.

Another embodiment comprises cyclosporin A, Dehymuls E®, and Sesame oil.

Another embodiment comprises cyclosporin A, DeSotan SMO®, and Sesame oil.

Another embodiment comprises cyclosporin A, DeSotan SMO-20®, and Sesame oil.

Another embodiment comprises cyclosporin A, DeSotan SMT®, and Sesame oil.

Another embodiment comprises cyclosporin A, DeSotan SMT-20®, and Sesame oil.

Another embodiment comprises cyclosporin A, Durfax 60®, and Sesame oil.

Another embodiment comprises cyclosporin A, Durfax 65®, and Sesame oil.

Another embodiment comprises cyclosporin A, Durfax 80®, and Sesame oil.

Another embodiment comprises cyclosporin A, Durtan 60®, and Sesame oil.

Another embodiment comprises cyclosporin A, Durtan 65®, and Sesame oil.

Another embodiment comprises cyclosporin A, Liposorb L®, and Sesame oil.

Another embodiment comprises cyclosporin A, Liposorb L-10®, and Sesame oil.

Another embodiment comprises cyclosporin A, Liposorb L-20®, and Sesame oil.

Another embodiment comprises cyclosporin A, Liposorb O®, and Sesame oil.

Another embodiment comprises cyclosporin A, Liposorb O-20®, and Sesame oil.

Another embodiment comprises cyclosporin A, Liposorb P®, and Sesame oil.

Another embodiment comprises cyclosporin A, Liposorb P-20®, and Sesame oil.

Another embodiment comprises cyclosporin A, Liposorb S®, and Sesame oil.

Another embodiment comprises cyclosporin A, Liposorb S-20®, and Sesame oil.

Another embodiment comprises cyclosporin A, Liposorb SQO®, and Sesame oil.

Another embodiment comprises cyclosporin A, Liposorb TO®, and Sesame oil.

Another embodiment comprises cyclosporin A, Liposorb TS®, and Sesame oil.

Another embodiment comprises cyclosporin A, Liposorb TS-20®, and Sesame oil.

Another embodiment comprises cyclosporin A, Lumisorb PS®, and Sesame oil.

Another embodiment comprises cyclosporin A, Lumisorb SMO (T)®, and Sesame oil.

Another embodiment comprises cyclosporin A, Lumisorb SMS K®, and Sesame oil.

Another embodiment comprises cyclosporin A, Lumisorb SSO®, and Sesame oil.

Another embodiment comprises cyclosporin A, Lumisorb STS K®, and Sesame oil.

Another embodiment comprises cyclosporin A, Lumisorb STT®, and Sesame oil.

Another embodiment comprises cyclosporin A, Magrabar SMO®, and Sesame oil.

Another embodiment comprises cyclosporin A, Magrabar SMO-VEG®, and Sesame oil.

Another embodiment comprises cyclosporin A, Magrabar SMT®, and Sesame oil.

Another embodiment comprises cyclosporin A, Magrabar STO®, and Sesame oil.

Another embodiment comprises cyclosporin A, Miracare BC-27®, and Sesame oil.

Another embodiment comprises cyclosporin A, Ritabate 20®, and Sesame oil.

Another embodiment comprises cyclosporin A, Ritabate 40®, and Sesame oil.

Another embodiment comprises cyclosporin A, Ritabate 60®, and Sesame oil.

Another embodiment comprises cyclosporin A, Ritabate 80®, and Sesame oil.

Another embodiment comprises cyclosporin A, T-Maz®, and Sesame oil.

Another embodiment comprises cyclosporin A, Tego SML®, and Sesame oil.

Another embodiment comprises cyclosporin A, Tego SML 20®, and Sesame oil.

Another embodiment comprises cyclosporin A, Tego SMO 80 V®, and Sesame oil.

Another embodiment comprises cyclosporin A, Tego SMO V®, and Sesame oil.

Another embodiment comprises cyclosporin A, Tego SMS®, and Sesame oil.

Another embodiment comprises cyclosporin A, Tego STO V®, and Sesame oil.

Another embodiment comprises cyclosporin A, Tween 21®, and Sesame oil.

Another embodiment comprises cyclosporin A, Tween 40®, and Sesame oil.

Another embodiment comprises cyclosporin A, Tween 60®, and Sesame oil.

Another embodiment comprises cyclosporin A, Tween 60 K®, and Sesame oil.

Another embodiment comprises cyclosporin A, Tween 61®, and Sesame oil.

Another embodiment comprises cyclosporin A, Tween 65®, and Sesame oil.

Another embodiment comprises cyclosporin A, Tween 80®, and Sesame oil.

Another embodiment comprises cyclosporin A, Tween 80 K®, and Sesame oil.

Another embodiment comprises cyclosporin A, Tween 81®, and Sesame oil.

Another embodiment comprises cyclosporin A, Tween 85®, and Sesame oil.

Another embodiment comprises cyclosporin A, a Sucrose or Glucose Ester, or Derivative thereof, and Sesame oil.

Another embodiment comprises cyclosporin A, DeSulf GOS-P-60WCG®, and Sesame oil.

Another embodiment comprises cyclosporin A, Glucam E-20 Distearate®, and Sesame oil.

Another embodiment comprises cyclosporin A, Glucamate DOE-120®, and Sesame oil.

Another embodiment comprises cyclosporin A, Glucamate SSE-20®, and Sesame oil.

Another embodiment comprises cyclosporin A, Glucate DO®, and Sesame oil.

Another embodiment comprises cyclosporin A, Glucate SS®, and Sesame oil.

Another embodiment comprises cyclosporin A, Glucopon 425 UP®, and Sesame oil.

Another embodiment comprises cyclosporin A, Isolan IS®, and Sesame oil.

Another embodiment comprises cyclosporin A, Mazon 40®, and Sesame oil.

Another embodiment comprises cyclosporin A, Montanov 82®, and Sesame oil.

Another embodiment comprises cyclosporin A, Montanov 202®, and Sesame oil.

Another embodiment comprises cyclosporin A, Montanov S®, and Sesame oil.

Another embodiment comprises cyclosporin A, Rheozan®, and Sesame oil.

Another embodiment comprises cyclosporin A, Simulsol AS 48®, and Sesame oil.

Another embodiment comprises cyclosporin A, Simulsol SL 4®, and Sesame oil.

Another embodiment comprises cyclosporin A, Simulsol SL 10®, and Sesame oil.

Another embodiment comprises cyclosporin A, Simulsol SL 11W®, and Sesame oil.

Another embodiment comprises cyclosporin A, Simulsol SL 55®, and Sesame oil.

Another embodiment comprises cyclosporin A, Suga Nate 100 and 160®, and Sesame oil.

Another embodiment comprises cyclosporin A, Tego Care 450®, and Sesame oil.

Another embodiment comprises cyclosporin A, Tego Care CG 90®, and Sesame oil.

Another embodiment comprises cyclosporin A, Tego Care PS®, and Sesame oil.

Another embodiment comprises cyclosporin A, Tegosoft PSE 141 G®, and Sesame oil.

Another embodiment comprises cyclosporin A, Tegotens G 826®, and Sesame oil.

Another embodiment comprises cyclosporin A, Triton BG-10 (70%)®, and Sesame oil.

Another embodiment comprises cyclosporin A, Triton CG-110 (60%)®, and Sesame oil.

Another embodiment comprises cyclosporin A, Wickenol 545®, and Sesame oil.

Another embodiment comprises cyclosporin A, an alcohol, and an oil having a Specific Gravity below 0.9.

Another embodiment comprises cyclosporin A, Diglycerol®, and an oil having a Specific Gravity below 0.9.

Another embodiment comprises cyclosporin A, Hetoxide GT-80®, and an oil having a Specific Gravity below 0.9.

Another embodiment comprises cyclosporin A, Lexemul BEO®, and an oil having a Specific Gravity below 0.9.

Another embodiment comprises cyclosporin A, Polyglycerol-3®, and an oil having a Specific Gravity below 0.9.

Another embodiment comprises cyclosporin A, Redicote E Series®, and an oil having a Specific Gravity below 0.9.

Another embodiment comprises cyclosporin A, Simulsol OX 1005L®, and an oil having a Specific Gravity below 0.9.

Another embodiment comprises cyclosporin A, Stanfax 567®, and an oil having a Specific Gravity below 0.9.

Another embodiment comprises cyclosporin A, TA-1618®, and an oil having a Specific Gravity below 0.9.

Another embodiment comprises cyclosporin A, Witconol H-31A®, and an oil having a Specific Gravity below 0.9.

Another embodiment comprises cyclosporin A, Standard Div.®, and an oil having a Specific Gravity below 0.9.

Another embodiment comprises cyclosporin A, an amine oxide, and an oil having a Specific Gravity below 0.9.

Another embodiment comprises cyclosporin A, AO-405®, and an oil having a Specific Gravity below 0.9.

Another embodiment comprises cyclosporin A, AO-455®, and an oil having a Specific Gravity below 0.9.

Another embodiment comprises cyclosporin A, AO 728 Special®, and an oil having a Specific Gravity below 0.9.

Another embodiment comprises cyclosporin A, Barlox 12®, and an oil having a Specific Gravity below 0.9.

Another embodiment comprises cyclosporin A, Barlox 14®, and an oil having a Specific Gravity below 0.9.

Another embodiment comprises cyclosporin A, Burcoxide Lo®, and an oil having a Specific Gravity below 0.9.

Another embodiment comprises cyclosporin A, Caloxamine LO®, and an oil having a Specific Gravity below 0.9.

Another embodiment comprises cyclosporin A, Chemoxide CAW®, and an oil having a Specific Gravity below 0.9.

Another embodiment comprises cyclosporin A, Chemoxide LM-30®, and an oil having a Specific Gravity below 0.9.

Another embodiment comprises cyclosporin A, Chembxide LO®, and an oil having a Specific Gravity below 0.9.

Another embodiment comprises cyclosporin A, Chemoxide MO®, and an oil having a Specific Gravity below 0.9.

Another embodiment comprises cyclosporin A, Colalux CAO-35®, and an oil having a Specific Gravity below 0.9.

Another embodiment comprises cyclosporin A, Colalux LO®, and an oil having a Specific Gravity below 0.9.

Another embodiment comprises cyclosporin A, DeMox CAPO®, and an oil having a Specific Gravity below 0.9.

Another embodiment comprises cyclosporin A, DeMox CSG-30®, and an oil having a Specific Gravity below 0.9.

Another embodiment comprises cyclosporin A, DeMox LAO®, and an oil having a Specific Gravity below 0.9.

Another embodiment comprises cyclosporin A, Emcol L®, and an oil having a Specific Gravity below 0.9.

Another embodiment comprises cyclosporin A, Empigen OB®, and an oil having a Specific Gravity below 0.9.

Another embodiment comprises cyclosporin A, Empigen OS/A®, and an oil having a Specific Gravity below 0.9.

Another embodiment comprises cyclosporin A, Foamox CDO®, and an oil having a Specific Gravity below 0.9.

Another embodiment comprises cyclosporin A, Foamox DMM®, and an oil having a Specific Gravity below 0.9.

Another embodiment comprises cyclosporin A, Foamox DMS®, and an oil having a Specific Gravity below 0.9.

Another embodiment comprises cyclosporin A, Genaminox KC®, and an oil having a Specific Gravity below 0.9.

Another embodiment comprises cyclosporin A, Genaminox LA®, and an oil having a Specific Gravity below 0.9.

Another embodiment comprises cyclosporin A, Hartofoam SAO®, and an oil having a Specific Gravity below 0.9.

Another embodiment comprises cyclosporin A, Hartox DMCD®, and an oil having a Specific Gravity below 0.9.

Another embodiment comprises cyclosporin A, Lipowax DAT®, and an oil having a Specific Gravity below 0.9.

Another embodiment comprises cyclosporin A, Lipowax PB Pastilles®, and an oil having a Specific Gravity below 0.9.

Another embodiment comprises cyclosporin A, Mackamine C8®, and an oil having a Specific Gravity below 0.9.

Another embodiment comprises cyclosporin A, Mackamine C10®, and an oil having a Specific Gravity below 0.9.

Another embodiment comprises cyclosporin A, Mackamine C14®, and an oil having a Specific Gravity below 0.9.

Another embodiment comprises cyclosporin A, Mackamine CAO®, and an oil having a Specific Gravity below 0.9.

Another embodiment comprises cyclosporin A, Mackamine CO®, and an oil having a Specific Gravity below 0.9.

Another embodiment comprises cyclosporin A, Mackamine LO®, and an oil having a Specific Gravity below 0.9.

Another embodiment comprises cyclosporin A, Mackamine O2®, and an oil having a Specific Gravity below 0.9.

Another embodiment comprises cyclosporin A, Mackamine SAO®, and an oil having a Specific Gravity below 0.9.

Another embodiment comprises cyclosporin A, Mackamine SO®, and an oil having a Specific Gravity below 0.9.

Another embodiment comprises cyclosporin A, Mazox KCAO®, and an oil having a Specific Gravity below 0.9.

Another embodiment comprises cyclosporin A, Monalac MO®, and an oil having a Specific Gravity below 0.9.

Another embodiment comprises cyclosporin A, Norfox LDA®, and an oil having a Specific Gravity below 0.9.

Another embodiment comprises cyclosporin A, Rhodamox LO®, and an oil having a Specific Gravity below 0.9.

Another embodiment comprises cyclosporin A, Schercamox C-AA®, and an oil having a Specific Gravity below 0.9.

Another embodiment comprises cyclosporin A, Schercamox DMA®, and an oil having a Specific Gravity below 0.9.

Another embodiment comprises cyclosporin A, Schercamox DML®, and an oil having a Specific Gravity below 0.9.

Another embodiment comprises cyclosporin A, Schercamox DMM®, and an oil having a Specific Gravity below 0.9.

Another embodiment comprises cyclosporin A, Schercamox DMS®, and an oil having a Specific Gravity below 0.9.

Another embodiment comprises cyclosporin A, Tegotens DC®, and an oil having a Specific Gravity below 0.9.

Another embodiment comprises cyclosporin A, Tomah AO-14-2®, and an oil having a Specific Gravity below 0.9.

Another embodiment comprises cyclosporin A, Triaminox CDO®, and an oil having a Specific Gravity below 0.9.

Another embodiment comprises cyclosporin A, a block polymer, and an oil having a Specific Gravity below 0.9.

Another embodiment comprises cyclosporin A, AL 2070®, and an oil having a Specific Gravity below 0.9.

Another embodiment comprises cyclosporin A, Antarox 17-R-2®, and an oil having a Specific Gravity below 0.9.

Another embodiment comprises cyclosporin A, Antarox 25-R-2®, and an oil having a Specific Gravity below 0.9.

Another embodiment comprises cyclosporin A, Antarox 31-R-1®, and an oil having a Specific Gravity below 0.9.

Another embodiment comprises cyclosporin A, Antarox P-84®, and an oil having a Specific Gravity below 0.9.

Another embodiment comprises cyclosporin A, Antarox P-104/H®, and an oil having a Specific Gravity below 0.9.

Another embodiment comprises cyclosporin A, Arnox BP-Series®, and an oil having a Specific Gravity below 0.9.

Another embodiment comprises cyclosporin A, Chemonic 435®, and an oil having a Specific Gravity below 0.9.

Another embodiment comprises cyclosporin A, Chemonic D-25®, and an oil having a Specific Gravity below 0.9.

Another embodiment comprises cyclosporin A, Chemonic PL Series®, and an oil having a Specific Gravity below 0.9.

Another embodiment comprises cyclosporin A, Ethox L-121®, and an oil having a Specific Gravity below 0.9.

Another embodiment comprises cyclosporin A, Ethox L-122®, and an oil having a Specific Gravity below 0.9.

Another embodiment comprises cyclosporin A, Genapol PF-10®, and an oil having a Specific Gravity below 0.9.

Another embodiment comprises cyclosporin A, Genapol PF-20®, and an oil having a Specific Gravity below 0.9.

Another embodiment comprises cyclosporin A, Genapol PF-40A®, and an oil having a Specific Gravity below 0.9.

Another embodiment comprises cyclosporin A, Norfox 2-LF®, and an oil having a Specific Gravity below 0.9.

Another embodiment comprises cyclosporin A, Simulsol NW 342®, and an oil having a Specific Gravity below 0.9.

Another embodiment comprises cyclosporin A, T-Det BP-1®, and an oil having a Specific Gravity below 0.9.

Another embodiment comprises cyclosporin A, T-Det XD®, and an oil having a Specific Gravity below 0.9.

Another embodiment comprises cyclosporin A, T-Det XH®, and an oil having a Specific Gravity below 0.9.

Another embodiment comprises cyclosporin A, Triton CF-32®, and an oil having a Specific Gravity below 0.9.

Another embodiment comprises cyclosporin A, Witconol 171®, and an oil having a Specific Gravity below 0.9.

Another embodiment comprises cyclosporin A, Witconol 324®, and an oil having a Specific Gravity below 0.9.

Another embodiment comprises cyclosporin A, Witconol 324D®, and an oil having a Specific Gravity below 0.9.

Another embodiment comprises cyclosporin A, Witconol PD-2000®, and an oil having a Specific Gravity below 0.9.

a Carboxylated Alcohol or Alkylphenol Ethoxylate, and an oil having a Specific Gravity below 0.9.

Another embodiment comprises cyclosporin A, Emcol CN-6®, and an oil having a Specific Gravity below 0.9.

Another embodiment comprises cyclosporin A, Ethcarb®, and an oil having a Specific Gravity below 0.9.

Another embodiment comprises cyclosporin A, Gemtex WNT-Conc®, and an oil having a Specific Gravity below 0.9.

Another embodiment comprises cyclosporin A, Incrodet TD7-C®, and an oil having a Specific Gravity below 0.9.

Another embodiment comprises cyclosporin A, Marlinat CM 105/80®, and an oil having a Specific Gravity below 0.9.

Another embodiment comprises cyclosporin A, Marlowet 1072®, and an oil having a Specific Gravity below 0.9.

Another embodiment comprises cyclosporin A, Marlowet 4530®, and an oil having a Specific Gravity below 0.9.

Another embodiment comprises cyclosporin A, Marlowet 4530 LF®, and an oil having a Specific Gravity below 0.9.

Another embodiment comprises cyclosporin A, Marlowet 4534®, and an oil having a Specific Gravity below 0.9.

Another embodiment comprises cyclosporin A, Marlowet 4538®, and an oil having a Specific Gravity below 0.9.

Another embodiment comprises cyclosporin A, Marlowet 4539®, and an oil having a Specific Gravity below 0.9.

Another embodiment comprises cyclosporin A, Marlowet 4539 LF®, and an oil having a Specific Gravity below 0.9.

Another embodiment comprises cyclosporin A, Marlowet 4541®, and an oil having a Specific Gravity below 0.9.

Another embodiment comprises cyclosporin A, Miranate LEC-80®, and an oil having a Specific Gravity below 0.9.

Another embodiment comprises cyclosporin A, Sandopan B®, and an oil having a Specific Gravity below 0.9.

Another embodiment comprises cyclosporin A, Sandopan B Modified®, and an oil having a Specific Gravity below 0.9.

Another embodiment comprises cyclosporin A, Sandopan LS-24 Gel®, and an oil having a Specific Gravity below 0.9.

Another embodiment comprises cyclosporin A, Surfine T-A®, and an oil having a Specific Gravity below 0.9.

Another embodiment comprises cyclosporin A, a Carboxylic Acid/Fatty Acid, and an oil having a Specific Gravity below 0.9.

Another embodiment comprises cyclosporin A, Colaterge RAM®, and an oil having a Specific Gravity below 0.9.

Another embodiment comprises cyclosporin A, Colatrope INC®, and an oil having a Specific Gravity below 0.9.

Another embodiment comprises cyclosporin A, Crodacid B®, and an oil having a Specific Gravity below 0.9.

Another embodiment comprises cyclosporin A, DeTrope CA-100®, and an oil having a Specific Gravity below 0.9.

Another embodiment comprises cyclosporin A, Latol MTO®, and an oil having a Specific Gravity below 0.9.

Another embodiment comprises cyclosporin A, Lumulse CC-33 K®, and an oil having a Specific Gravity below 0.9.

Another embodiment comprises cyclosporin A, Mulls 2218®, and an oil having a Specific Gravity below 0.9.

Another embodiment comprises cyclosporin A, OL-600®, and an oil having a Specific Gravity below 0.9.

Another embodiment comprises cyclosporin A, OL-800®, and an oil having a Specific Gravity below 0.9.

Another embodiment comprises cyclosporin A, R-910®, and an oil having a Specific Gravity below 0.9.

Another embodiment comprises cyclosporin A, S-210®, and an oil having a Specific Gravity below 0.9.

Another embodiment comprises cyclosporin A, Sandopan DTC Acid®, and an oil having a Specific Gravity below 0.9.

Another embodiment comprises cyclosporin A, Sandopan LS 24 N®, and an oil having a Specific Gravity below 0.9.

Another embodiment comprises cyclosporin A, Sandopan MA-18®, and an oil having a Specific Gravity below 0.9.

Another embodiment comprises cyclosporin A, an Ethoxylated Alcohol, and an oil having a Specific Gravity below 0.9.

Another embodiment comprises cyclosporin A, Adsee 799®, and an oil having a Specific Gravity below 0.9.

Another embodiment comprises cyclosporin A, Adsee 799®, and an oil having a Specific Gravity below 0.9.

Another embodiment comprises cyclosporin A, Alfonic 610-3.5®, and an oil having a Specific Gravity below 0.9.

Another embodiment comprises cyclosporin A, Alfonic 810-2®, and an oil having a Specific Gravity below 0.9.

Another embodiment comprises cyclosporin A, Alfonic 810-6®, and an oil having a Specific Gravity below 0.9.

Another embodiment comprises cyclosporin A, Alfonic 1012-3®, and an oil having a Specific Gravity below 0.9.

Another embodiment comprises cyclosporin A, Alfonic 1012-5®, and an oil having a Specific Gravity below 0.9.

Another embodiment comprises cyclosporin A, Alfonic 1216CO-1.5®, and an oil having a Specific Gravity below 0.9.

Another embodiment comprises cyclosporin A, Alfonic 1216CO-7®, and an oil having a Specific Gravity below 0.9.

Another embodiment comprises cyclosporin A, Alfonic 1412-3®, and an oil having a Specific Gravity below 0.9.

Another embodiment comprises cyclosporin A, Alfonic 1412-7®, and an oil having a Specific Gravity below 0.9.

Another embodiment comprises cyclosporin A, Arlasolve 200®, and an oil having a Specific Gravity below 0.9.

Another embodiment comprises cyclosporin A, Arlasolve 200 Liquid®, and an oil having a Specific Gravity below 0.9.

Another embodiment comprises cyclosporin A, Armix 180-C®, and an oil having a Specific Gravity below 0.9.

Another embodiment comprises cyclosporin A, Armix 183®, and an oil having a Specific Gravity below 0.9.

Another embodiment comprises cyclosporin A, Armul 2404®, and an oil having a Specific Gravity below 0.9.

Another embodiment comprises cyclosporin A, Armul 2404®, and an oil having a Specific Gravity below 0.9.

Another embodiment comprises cyclosporin A, Atlas EMJ-C®, and an oil having a Specific Gravity below 0.9.

Another embodiment comprises cyclosporin A, Atlas G-2109®, and an oil having a Specific Gravity below 0.9.

Another embodiment comprises cyclosporin A, Atlas G-3886®, and an oil having a Specific Gravity below 0.9.

Another embodiment comprises cyclosporin A, Atlas G-3890®, and an oil having a Specific Gravity below 0.9.

Another embodiment comprises cyclosporin A, Bio Soft E-200®, and an oil having a Specific Gravity below 0.9.

Another embodiment comprises cyclosporin A, Bio Soft E-300®, and an oil having a Specific Gravity below 0.9.

Another embodiment comprises cyclosporin A, Bio Soft E-400®, and an oil having a Specific Gravity below 0.9.

Another embodiment comprises cyclosporin A, Bio Soft EN 600®, and an oil having a Specific Gravity below 0.9.

Another embodiment comprises cyclosporin A, Bio Soft TD-400®, and an oil having a Specific Gravity below 0.9.

Another embodiment comprises cyclosporin A, Bio Soft TD-630®, and an oil having a Specific Gravity below 0.9.

Another embodiment comprises cyclosporin A, Brij 30®, and an oil having a Specific Gravity below 0.9.

Another embodiment comprises cyclosporin A, Brij 52®, and an oil having a Specific Gravity below 0.9.

Another embodiment comprises cyclosporin A, Brij 56§, and an oil having a Specific Gravity below 0.9.

Another embodiment comprises cyclosporin A, Brij 58®, and an oil having a Specific Gravity below 0.9.

Another embodiment comprises cyclosporin A, Brij 72®, and an oil having a Specific Gravity below 0.9.

Another embodiment comprises cyclosporin A, Brij 76®, and an oil having a Specific Gravity below 0.9.

Another embodiment comprises cyclosporin A, Brij 78®, and an oil having a Specific Gravity below 0.9.

Another embodiment comprises cyclosporin A, Brij 93®, and an oil having a Specific Gravity below 0.9.

Another embodiment comprises cyclosporin A, Brij 97®, and an oil having a Specific Gravity below 0.9.

Another embodiment comprises cyclosporin A, Brij 98®, and an oil having a Specific Gravity below 0.9.

Another embodiment comprises cyclosporin A, Brij 700®, and an oil having a Specific Gravity below 0.9.

Another embodiment comprises cyclosporin A, Brij 700 S®, and an oil having a Specific Gravity below 0.9.

Another embodiment comprises cyclosporin A, Brij 721®, and an oil having a Specific Gravity below 0.9.

Another embodiment comprises cyclosporin A, Brij 721 S®, and an oil having a Specific Gravity below 0.9.

Another embodiment comprises cyclosporin A, Burcoterge CDG®, and an oil having a Specific Gravity below 0.9.

Another embodiment comprises cyclosporin A, Canasol AT 600®, and an oil having a Specific Gravity below 0.9.

Another embodiment comprises cyclosporin A, Canasol AT 800®, and an oil having a Specific Gravity below 0.9.

Another embodiment comprises cyclosporin A, Canasol AT 1200®, and an oil having a Specific Gravity below 0.9.

Another embodiment comprises cyclosporin A, Canasol BJ 35®, and an oil having a Specific Gravity below 0.9.

Another embodiment comprises cyclosporin A, Canasol BJ 36®, and an oil having a Specific Gravity below 0.9.

Another embodiment comprises cyclosporin A, Canasol BJ 52®, and an oil having a Specific Gravity below 0.9.

Another embodiment comprises cyclosporin A, Canasol BJ 58®, and an oil having a Specific Gravity below 0.9.

Another embodiment comprises cyclosporin A, Canasol BJ 72®, and an oil having a Specific Gravity below 0.9.

Another embodiment comprises cyclosporin A, Canasol BJ 78®, and an oil having a Specific Gravity below 0.9.

Another embodiment comprises cyclosporin A, Canasol BJ 98®, and an oil having a Specific Gravity below 0.9.

Another embodiment comprises cyclosporin A, Canasol BJ 307®, and an oil having a Specific Gravity below 0.9.

Another embodiment comprises cyclosporin A, Cerfak 1400®, and an oil having a Specific Gravity below 0.9.

Another embodiment comprises cyclosporin A, Cetomacrogol 1000 BP®, and an oil having a Specific Gravity below 0.9.

Another embodiment comprises cyclosporin A, Chemonic C-2®, and an oil having a Specific Gravity below 0.9.

Another embodiment comprises cyclosporin A, Chemonic C-10®, and an oil having a Specific Gravity below 0.9.

Another embodiment comprises cyclosporin A, Chemonic C-20®, and an oil having a Specific Gravity below 0.9.

Another embodiment comprises cyclosporin A, Chemonic CT-12®, and an oil having a Specific Gravity below 0.9.

Another embodiment comprises cyclosporin A, Chemonic CT-20®, and an oil having a Specific Gravity below 0.9.

Another embodiment comprises cyclosporin A, Chemonic CT-30®, and an oil having a Specific Gravity below 0.9.

Another embodiment comprises cyclosporin A, Chemonic CT-55®, and an oil having a Specific Gravity below 0.9.

Another embodiment comprises cyclosporin A, Chemonic G-7®, and an oil having a Specific Gravity below 0.9.

Another embodiment comprises cyclosporin A, Chemonic G-26®, and an oil having a Specific Gravity below 0.9.

Another embodiment comprises cyclosporin A, Chemonic L-4®, and an oil having a Specific Gravity below 0.9.

Another embodiment comprises cyclosporin A, Chemonic L-7®, and an oil having a Specific Gravity below 0.9.

Another embodiment comprises cyclosporin A, Chemonic L-12®, and an oil having a Specific Gravity below 0.9.

Another embodiment comprises cyclosporin A, Chemonic L-23®, and an oil having a Specific Gravity below 0.9.

Another embodiment comprises cyclosporin A, Chemonic O-2®, and an oil having a Specific Gravity below 0.9.

Another embodiment comprises cyclosporin A, Chemonic O-5®, and an oil having a Specific Gravity below 0.9.

Another embodiment comprises cyclosporin A, Chemonic O-10®, and an oil having a Specific Gravity below 0.9.

Another embodiment comprises cyclosporin A, Chemonic O-20®, and an oil having a Specific Gravity below 0.9.

Another embodiment comprises cyclosporin A, Chemonic S-2®, and an oil having a Specific Gravity below 0.9.

Another embodiment comprises cyclosporin A, Chemonic S-10®, and an oil having a Specific Gravity below 0.9.

Another embodiment comprises cyclosporin A, Chemonic S-20®, and an oil having a Specific Gravity below 0.9.

Another embodiment comprises cyclosporin A, Colamulse FE®, and an oil having a Specific Gravity below 0.9.

Another embodiment comprises cyclosporin A, Cremophor A 20®, and an oil having a Specific Gravity below 0.9.

Another embodiment comprises cyclosporin A, Cremophor SA 2®, and an oil having a Specific Gravity below 0.9.

Another embodiment comprises cyclosporin A, Dehydol 100®, and an oil having a Specific Gravity below 0.9.

Another embodiment comprises cyclosporin A, Dehydol O-4®, and an oil having a Specific Gravity below 0.9.

Another embodiment comprises cyclosporin A, Delonic C-18®, and an oil having a Specific Gravity below 0.9.

Another embodiment comprises cyclosporin A, DeSonic 6T®, and an oil having a Specific Gravity below 0.9.

Another embodiment comprises cyclosporin A, DeSonic 9D®, and an oil having a Specific Gravity below 0.9.

Another embodiment comprises cyclosporin A, DeSonic 9T®, and an oil having a Specific Gravity below 0.9.

Another embodiment comprises cyclosporin A, DeSonic 12D®, and an oil having a Specific Gravity below 0.9.

Another embodiment comprises cyclosporin A, DeSonic 12T®, and an oil having a Specific Gravity below 0.9.

Another embodiment comprises cyclosporin A, DeSonic 15T®, and an oil having a Specific Gravity below 0.9.

Another embodiment comprises cyclosporin A, DeSonic TDA-9®, and an oil having a Specific Gravity below 0.9.

Another embodiment comprises cyclosporin A, DeThox GLG-7®, and an oil having a Specific Gravity below 0.9.

Another embodiment comprises cyclosporin A, DeThox GLG-26®, and an oil having a Specific Gravity below 0.9.

Another embodiment comprises cyclosporin A, DeThox LA-4®, and an oil having a Specific Gravity below 0.9.

Another embodiment comprises cyclosporin A, DeThox LA-23®, and an oil having a Specific Gravity below 0.9.

Another embodiment comprises cyclosporin A, DeThox SA-80®, and an oil having a Specific Gravity below 0.9.

Another embodiment comprises cyclosporin A, Disponil O5®, and an oil having a Specific Gravity below 0.9.

Another embodiment comprises cyclosporin A, Eccoterge EO-41B®, and an oil having a Specific Gravity below 0.9.

Another embodiment comprises cyclosporin A, Empilan KA2.5/90®, and an oil having a Specific Gravity below 0.9.

Another embodiment comprises cyclosporin A, Empilan KA5/90®, and an oil having a Specific Gravity below 0.9.

Another embodiment comprises cyclosporin A, Empilan KM-20®, and an oil having a Specific Gravity below 0.9.

Another embodiment comprises cyclosporin A, Empilan KM-50®, and an oil having a Specific Gravity below 0.9.

Another embodiment comprises cyclosporin A, Empilan L-23®, and an oil having a Specific Gravity below 0.9.

Another embodiment comprises cyclosporin A, Ethylan 25-3®, and an oil having a Specific Gravity below 0.9.

Another embodiment comprises cyclosporin A, Ethylan 1204®, and an oil having a Specific Gravity below 0.9.

Another embodiment comprises cyclosporin A, Ethylan DA-4®, and an oil having a Specific Gravity below 0.9.

Another embodiment comprises cyclosporin A, Ethylan LA-230®, and an oil having a Specific Gravity below 0.9.

Another embodiment comprises cyclosporin A, Ethylan SN®, and an oil having a Specific Gravity below 0.9.

Another embodiment comprises cyclosporin A, Ethylan TD-60®, and an oil having a Specific Gravity below 0.9.

Another embodiment comprises cyclosporin A, Ethylan TD-100®, and an oil having a Specific Gravity below 0.9.

Another embodiment comprises cyclosporin A, Ethylan TD-1407®, and an oil having a Specific Gravity below 0.9.

Another embodiment comprises cyclosporin A, Eumulgin B1®, and an oil having a Specific Gravity below 0.9.

Another embodiment comprises cyclosporin A, Eumulgin B2®, and an oil having a Specific Gravity below 0.9.

Another embodiment comprises cyclosporin A, Eumulgin B3®, and an oil having a Specific Gravity below 0.9.

Another embodiment comprises cyclosporin A, Eumulgin O-10®, and an oil having a Specific Gravity below 0.9.

Another embodiment comprises cyclosporin A, Flo Mo 80/20®, and an oil having a Specific Gravity below 0.9.

Another embodiment comprises cyclosporin A, Flo Mo Low Foam®, and an oil having a Specific Gravity below 0.9.

Another embodiment comprises cyclosporin A, Forlan C-24®, and an oil having a Specific Gravity below 0.9.

Another embodiment comprises cyclosporin A, Genapol 1454®, and an oil having a Specific Gravity below 0.9.

Another embodiment comprises cyclosporin A, Genapol BA-020®, and an oil having a Specific Gravity below 0.9.

Another embodiment comprises cyclosporin A, Genapol BA-040®, and an oil having a Specific Gravity below 0.9.

Another embodiment comprises cyclosporin A, Genapol C-100®, and an oil having a Specific Gravity below 0.9.

Another embodiment comprises cyclosporin A, Genapol DA 060®, and an oil having a Specific Gravity below 0.9.

Another embodiment comprises cyclosporin A, Genapol HS 020®, and an oil having a Specific Gravity below 0.9.

Another embodiment comprises cyclosporin A, Genapol HS 200®, and an oil having a Specific Gravity below 0.9.

Another embodiment comprises cyclosporin A, Genapol ID-040®, and an oil having a Specific Gravity below 0.9.

Another embodiment comprises cyclosporin A, Genapol ID-060®, and an oil having a Specific Gravity below 0.9.

Another embodiment comprises cyclosporin A, Genapol ID-090®, and an oil having a Specific Gravity below 0.9.

Another embodiment comprises cyclosporin A, Genapol LA 010®, and an oil having a Specific Gravity below 0.9.

Another embodiment comprises cyclosporin A, Genapol LA 020®, and an oil having a Specific Gravity below 0.9.

Another embodiment comprises cyclosporin A, Genapol LA 030®; and an oil having a Specific Gravity below 0.9.

Another embodiment comprises cyclosporin A, Genapol LA 040®, and an oil having a Specific Gravity below 0.9.

Another embodiment comprises cyclosporin A, Genapol LA 050®, and an oil having a Specific Gravity below 0.9.

Another embodiment comprises cyclosporin A, Genapol LA 060®, and an oil having a Specific Gravity below 0.9.

Another embodiment comprises cyclosporin A, Genapol LA 070®, and an oil having a Specific Gravity below 0.9.

Another embodiment comprises cyclosporin A, Genapol LA 070S®, and an oil having a Specific Gravity below 0.9.

Another embodiment comprises cyclosporin A, Genapol LA 230®, and an oil having a Specific Gravity below 0.9.

Another embodiment comprises cyclosporin A, Genapol O020®, and an oil having a Specific Gravity below 0.9.

Another embodiment comprises cyclosporin A, Genapol O050®, and an oil having a Specific Gravity below 0.9.

Another embodiment comprises cyclosporin A, Genapol O 100®, and an oil having a Specific Gravity below 0.9.

Another embodiment comprises cyclosporin A, Genapol O 200®, and an oil having a Specific Gravity below 0.9.

Another embodiment comprises cyclosporin A, Genapol SA 030®, and an oil having a Specific Gravity below 0.9.

Another embodiment comprises cyclosporin A, Genapol SA 120®, and an oil having a Specific Gravity below 0.9.

Another embodiment comprises cyclosporin A, Genapol T-020®, and an oil having a Specific Gravity below 0.9.

Another embodiment comprises cyclosporin A, Genapol UD-030®, and an oil having a Specific Gravity below 0.9.

Another embodiment comprises cyclosporin A, Genapol UD-050®, and an oil having a Specific Gravity below 0.9.

Another embodiment comprises cyclosporin A, Genapol UD-070®, and an oil having a Specific Gravity below 0.9.

Another embodiment comprises cyclosporin A, Genapol UD-079®, and an oil having a Specific Gravity below 0.9.

Another embodiment comprises cyclosporin A, Genapol UD-080®, and an oil having a Specific Gravity below 0.9.

Another embodiment comprises cyclosporin A, Genapol UD-110®, and an oil having a Specific Gravity below 0.9.

Another embodiment comprises cyclosporin A, Genapol X 030®, and an oil having a Specific Gravity below 0.9.

Another embodiment comprises cyclosporin A, Genapol X 050®, and an oil having a Specific Gravity below 0.9.

Another embodiment comprises cyclosporin A, Genapbl X 060®, and an oil having a Specific Gravity below 0.9.

Another embodiment comprises cyclosporin A, Genapol X 070®, and an oil having a Specific Gravity below 0.9.

Another embodiment comprises cyclosporin A, Genapol X 080®, and an oil having a Specific Gravity below 0.9.

Another embodiment comprises cyclosporin A, Genapol X 100®, and an oil having a Specific Gravity below 0.9.

Another embodiment comprises cyclosporin A, Genapol X159®, and an oil having a Specific Gravity below 0.9.

Another embodiment comprises cyclosporin A, Generol 122 E5®, and an oil having a Specific Gravity below 0.9.

Another embodiment comprises cyclosporin A, Generol 122 E25®, and an oil having a Specific Gravity below 0.9.

Another embodiment comprises cyclosporin A, Hostacerin T-3®, and an oil having a Specific Gravity below 0.9.

Another embodiment comprises cyclosporin A, Iconol LF 110®, and an oil having a Specific Gravity below 0.9.

Another embodiment comprises cyclosporin A, Incropol CS-20®, and an oil having a Specific Gravity below 0.9.

Another embodiment comprises cyclosporin A, Lexemul CS-20®, and an oil having a Specific Gravity below 0.9.

Another embodiment comprises cyclosporin A, Liponic EG-1®, and an oil having a Specific Gravity below 0.9.

Another embodiment comprises cyclosporin A, Lipowax D®, and an oil having a Specific Gravity below 0.9.

Another embodiment comprises cyclosporin A, Lipowax G®, and an oil having a Specific Gravity below 0.9.

Another embodiment comprises cyclosporin A, Lipowax NI®, and an oil having a Specific Gravity below 0.9.

Another embodiment comprises cyclosporin A, Lipowax P®, and an oil having a Specific Gravity below 0.9.

Another embodiment comprises cyclosporin A, Lipowax P-31®, and an oil having a Specific Gravity below 0.9.

Another embodiment comprises cyclosporin A, Lipowax PR®, and an oil having a Specific Gravity below 0.9.

Another embodiment comprises cyclosporin A, Lumulse CS-20®, and an oil having a Specific Gravity below 0.9.

Another embodiment comprises cyclosporin A, Macol CSA-20®, and an oil having a Specific Gravity below 0.9.

Another embodiment comprises cyclosporin A, Marlox B 24/50®, and an oil having a Specific Gravity below 0.9.

Another embodiment comprises cyclosporin A, Mazawet 77®, and an oil having a Specific Gravity below 0.9.

Another embodiment comprises cyclosporin A, Norfox 1713®, and an oil having a Specific Gravity below 0.9.

Another embodiment comprises cyclosporin A, Norfox 2579®, and an oil having a Specific Gravity below 0.9.

Another embodiment comprises cyclosporin A, Norfox Lo Foam®, and an oil having a Specific Gravity below 0.9.

Another embodiment comprises cyclosporin A, Promulgen D®, and an oil having a Specific Gravity below 0.9.

Another embodiment comprises cyclosporin A, Promulgen G®, and an oil having a Specific Gravity below 0.9.

Another embodiment comprises cyclosporin A, Renex 30®, and an oil having a Specific Gravity below 0.9.

Another embodiment comprises cyclosporin A, Renex 36®, and an oil having a Specific Gravity below 0.9.

Another embodiment comprises cyclosporin A, Rhodasurf A 24®, and an oil having a Specific Gravity below 0.9.

Another embodiment comprises cyclosporin A, Rhodasurf AAE-10®, and an oil having a Specific Gravity below 0.9.

Another embodiment comprises cyclosporin A, Rhodasurf BEH-25®, and an oil having a Specific Gravity below 0.9.

Another embodiment comprises cyclosporin A, Rhodasurf BEH-40®, and an oil having a Specific Gravity below 0.9.

Another embodiment comprises cyclosporin A, Rhodasurf DA 530®, and an oil having a Specific Gravity below 0.9.

Another embodiment comprises cyclosporin A, Rhodasurf DA 630®, and an oil having a Specific Gravity below 0.9.

Another embodiment comprises cyclosporin A, Rhodasurf DA 639®, and an oil having a Specific Gravity below 0.9.

Another embodiment comprises cyclosporin A, Rhodasurf LAN-23®, and an oil having a Specific Gravity below 0.9.

Another embodiment comprises cyclosporin A, Rhodasurf ON-870®, and an oil having a Specific Gravity below 0.9.

Another embodiment comprises cyclosporin A, Rhodasurf ON-877®, and an oil having a Specific Gravity below 0.9.

Another embodiment comprises cyclosporin A, Rhodasurf TB-970 FLK®, and an oil having a Specific Gravity below 0.9.

Another embodiment comprises cyclosporin A, Ritacet-20®, and an oil having a Specific Gravity below 0.9.

Another embodiment comprises cyclosporin A, Ritachol 1000®, and an oil having a Specific Gravity below 0.9.

Another embodiment comprises cyclosporin A, Ritachol 2000®, and an oil having a Specific Gravity below 0.9.

Another embodiment comprises cyclosporin A, Ritachol 5000®, and an oil having a Specific Gravity below 0.9.

Another embodiment comprises cyclosporin A, Ritox 35®, and an oil having a Specific Gravity below 0.9.

Another embodiment comprises cyclosporin A, Surfonic DA-4®, and an oil having a Specific Gravity below 0.9.

Another embodiment comprises cyclosporin A, Surfonic DA-6®, and an oil having a Specific Gravity below 0.9.

Another embodiment comprises cyclosporin A, Surfonic L46-7®, and an oil having a Specific Gravity below 0.9.

Another embodiment comprises cyclosporin A, Surfonic POA®, and an oil having a Specific Gravity below 0.9.

Another embodiment comprises cyclosporin A, Synthrapol KB®, and an oil having a Specific Gravity below 0.9.

Another embodiment comprises cyclosporin A, Teginacid®, and an oil having a Specific Gravity below 0.9.

Another embodiment comprises cyclosporin A, Teginacid C®, and an oil having a Specific Gravity below 0.9.

Another embodiment comprises cyclosporin A, Tegotens EC 11®, and an oil having a Specific Gravity below 0.9.

Another embodiment comprises cyclosporin A, Tinegal NA®, and an oil having a Specific Gravity below 0.9.

Another embodiment comprises cyclosporin A, Tomadol 400®, and an oil having a Specific Gravity below 0.9.

Another embodiment comprises cyclosporin A, Tomadol 600®, and an oil having a Specific Gravity below 0.9.

Another embodiment comprises cyclosporin A, Tomadol 900®, and an oil having a Specific Gravity below 0.9.

Another embodiment comprises cyclosporin A, Uniperol O®, and an oil having a Specific Gravity below 0.9.

Another embodiment comprises cyclosporin A, Witconol SN Series®, and an oil having a Specific Gravity below 0.9.

Another embodiment comprises cyclosporin A, an Ethoxylated Alkylphenol, and an oil having a Specific Gravity below 0.9.

Another embodiment comprises cyclosporin A, Antarox LF-222®, and an oil having a Specific Gravity below 0.9.

Another embodiment comprises cyclosporin A, Atlox 775®, and an oil having a Specific Gravity below 0.9.

Another embodiment comprises cyclosporin A, Caloxylate N-9®, and an oil having a Specific Gravity below 0.9.

Another embodiment comprises cyclosporin A, Canasol NF-1000®, and an oil having a Specific Gravity below 0.9.

Another embodiment comprises cyclosporin A, Canasol NF-3000®, and an oil having a Specific Gravity below 0.9.

Another embodiment comprises cyclosporin A, Canasol NF-3070®, and an oil having a Specific Gravity below 0.9.

Another embodiment comprises cyclosporin A, Canasol OF 1670®, and an oil having a Specific Gravity below 0.9.

Another embodiment comprises cyclosporin A, Canasol OF 2570®, and an oil having a Specific Gravity below 0.9.

Another embodiment comprises cyclosporin A, Canasol OF 4070®, and an oil having a Specific Gravity below 0.9.

Another embodiment comprises cyclosporin A, Chemax DNP-8®, and an oil having a Specific Gravity below 0.9.

Another embodiment comprises cyclosporin A, Chemax DNP-18®, and an oil having a Specific Gravity below 0.9.

Another embodiment comprises cyclosporin A, Chemax DNP-150/50®, and an oil having a Specific Gravity below 0.9.

Another embodiment comprises cyclosporin A, DeSonic 1.5N®, and an oil having a Specific Gravity below 0.9.

Another embodiment comprises cyclosporin A, DeSonic 4N®, and an oil having a Specific Gravity below 0.9.

Another embodiment comprises cyclosporin A, DeSonic 5N®, and an oil having a Specific Gravity below 0.9.

Another embodiment comprises cyclosporin A, DeSonic 6D®, and an oil having a Specific Gravity below 0.9.

Another embodiment comprises cyclosporin A, DeSonic 6N®, and an oil having a Specific Gravity below 0.9.

Another embodiment comprises cyclosporin A, DeSonic 7N®, and an oil having a Specific Gravity below 0.9.

Another embodiment comprises cyclosporin A, DeSonic 9N®, and an oil having a Specific Gravity below 0.9.

Another embodiment comprises cyclosporin A, DeSonic 10D®, and an oil having a Specific Gravity below 0.9.

Another embodiment comprises cyclosporin A, DeSonic 11N®, and an oil having a Specific Gravity below 0.9.

Another embodiment comprises cyclosporin A, DeSonic 12N®, and an oil having a Specific Gravity below 0.9.

Another embodiment comprises cyclosporin A, DeSonic 15N®, and an oil having a Specific Gravity below 0.9.

Another embodiment comprises cyclosporin A, DeSonic 20N®, and an oil having a Specific Gravity below 0.9.

Another embodiment comprises cyclosporin A, Eccoscour RC®, and an oil having a Specific Gravity below 0.9.

Another embodiment comprises cyclosporin A, Eccoterge EO-100®, and an oil having a Specific Gravity below 0.9.

Another embodiment comprises cyclosporin A, Emulsifier 632/90%®, and an oil having a Specific Gravity below 0.9.

Another embodiment comprises cyclosporin A, Geronol AG-821®, and an oil having a Specific Gravity below 0.9.

Another embodiment comprises cyclosporin A, Gradonic N-95®, and an oil having a Specific Gravity below 0.9.

Another embodiment comprises cyclosporin A, Hetoxide NP-4®, and an oil having a Specific Gravity below 0.9.

Another embodiment comprises cyclosporin A, Hetoxide NP-30®, and an oil having a Specific Gravity below 0.9.

Another embodiment comprises cyclosporin A, Hostapal N-100®, and an oil having a Specific Gravity below 0.9.

Another embodiment comprises cyclosporin A, Hostapal N-110®, and an oil having a Specific Gravity below 0.9.

Another embodiment comprises cyclosporin A, Igepal CTA-639W®, and an oil having a Specific Gravity below 0.9.

Another embodiment comprises cyclosporin A, Igepal DAP-9®, and an oil having a Specific Gravity below 0.9.

Another embodiment comprises cyclosporin A, Igepal OD-410®, and an oil having a Specific Gravity below 0.9.

Another embodiment comprises cyclosporin A, Igepal SS-837®, and an oil having a Specific Gravity below 0.9.

Another embodiment comprises cyclosporin A, Lipocol NP-9 USP®, and an oil having a Specific Gravity below 0.9.

Another embodiment comprises cyclosporin A, Macol DNP-10®, and an oil having a Specific Gravity below 0.9.

Another embodiment comprises cyclosporin A, Marlophen NP 5®, and an oil having a Specific Gravity below 0.9.

Another embodiment comprises cyclosporin A, Marlophen P 1®, and an oil having a Specific Gravity below 0.9.

Another embodiment comprises cyclosporin A, Surfonic NB®, and an oil having a Specific Gravity below 0.9.

Another embodiment comprises cyclosporin A, Surfonic OPB-307®, and an oil having a Specific Gravity below 0.9.

Another embodiment comprises cyclosporin A, Surfonic OPB-407®, and an oil having a Specific Gravity below 0.9.

Another embodiment comprises cyclosporin A, Syn Fac 334®, and an oil having a Specific Gravity below 0.9.

Another embodiment comprises cyclosporin A, Syn Fac 8216®, and an oil having a Specific Gravity below 0.9.

Another embodiment comprises cyclosporin A, Triton N-57®, and an oil having a Specific Gravity below 0.9.

Another embodiment comprises cyclosporin A, Trycol 6956®, and an oil having a Specific Gravity below 0.9.

Another embodiment comprises cyclosporin A, Trycol 6961®, and an oil having a Specific Gravity below 0.9.

Another embodiment comprises cyclosporin A, Trycol 6964®, and an oil having a Specific Gravity below 0.9.

Another embodiment comprises cyclosporin A, Trycol 6969®, and an oil having a Specific Gravity below 0.9.

Another embodiment comprises cyclosporin A, Trycol 6974®, and an oil having a Specific Gravity below 0.9.

Another embodiment comprises cyclosporin A, Witbreak DRB-127®, and an oil having a Specific Gravity below 0.9.

Another embodiment comprises cyclosporin A, Witbreak DRB-127®, and an oil having a Specific Gravity below 0.9.

Another embodiment comprises cyclosporin A, Witconol NP Series®, and an oil having a Specific Gravity below 0.9.

Another embodiment comprises cyclosporin A, an Ethoxylated Aryl Phenol, and an oil having a Specific Gravity below 0.9.

Another embodiment comprises cyclosporin A, Soprophor BSU®, and an oil having a Specific Gravity below 0.9.

Another embodiment comprises cyclosporin A, Soprophor CY/8®, and an oil having a Specific Gravity below 0.9.

Another embodiment comprises cyclosporin A, Soprophor S/25®, and an oil having a Specific Gravity below 0.9.

Another embodiment comprises cyclosporin A, Witconol NIO®, and an oil having a Specific Gravity below 0.9.

Another embodiment comprises cyclosporin A, Witconol NIW®, and an oil having a Specific Gravity below 0.9.

Another embodiment comprises cyclosporin A, Witconol S-100®, and an oil having a Specific Gravity below 0.9.

Another embodiment comprises cyclosporin A, an Ethoxylated Fatty Acid, and an oil having a Specific Gravity below 0.9.

Another embodiment comprises cyclosporin A, Aldo PGHMS®, and an oil having a Specific Gravity below 0.9.

Another embodiment comprises cyclosporin A, Alkamuls TO-15/HR®, and an oil having a Specific Gravity below 0.9.

Another embodiment comprises cyclosporin A, Armotan AL-69-66®, and an oil having a Specific Gravity below 0.9.

Another embodiment comprises cyclosporin A, Cerasynt 840®, and an oil having a Specific Gravity below 0.9.

Another embodiment comprises cyclosporin A, Cerasynt 945®, and an oil having a Specific Gravity below 0.9.

Another embodiment comprises cyclosporin A, Crystal Inhibitor No. 5®, and an oil having a Specific Gravity below 0.9.

Another embodiment comprises cyclosporin A, DeThox Acid L-9®, and an oil having a Specific Gravity below 0.9.

Another embodiment comprises cyclosporin A, DeThox Acid S-8®, and an oil having a Specific Gravity below 0.9.

Another embodiment comprises cyclosporin A, Ethofat 242/25®, and an oil having a-Specific Gravity below 0.9.

Another embodiment comprises cyclosporin A, Hydropalat 65®, and an oil having a Specific Gravity below 0.9.

Another embodiment comprises cyclosporin A, Lipo EGMS®, and an oil having a Specific Gravity below 0.9.

Another embodiment comprises cyclosporin A, Lipopeg 2 DL®, and an oil having a Specific Gravity below 0.9.

Another embodiment comprises cyclosporin A, Lipopeg 4 DL®, and an oil having a Specific Gravity below 0.9.

Another embodiment comprises cyclosporin A, Lipopeg 4-L®, and an oil having a Specific Gravity below 0.9.

Another embodiment comprises cyclosporin A, Lipopeg 39-S®, and an oil having a Specific Gravity below 0.9.

Another embodiment comprises cyclosporin A, Lipopeg 4-S®, and an oil having a Specific Gravity below 0.9.

Another embodiment comprises cyclosporin A, Lipopeg 10-S®, and an oil having a Specific Gravity below 0.9.

Another embodiment comprises cyclosporin A, Lipopeg 100-S®, and an oil having a Specific Gravity below 0.9.

Another embodiment comprises cyclosporin A, Lipopeg 6000 DS®, and an oil having a Specific Gravity below 0.9.

Another embodiment comprises cyclosporin A, Lumulse 40-L®, and an oil having a Specific Gravity below 0.9.

Another embodiment comprises cyclosporin A, Lumulse 40-S®, and an oil having a Specific Gravity below 0.9.

Another embodiment comprises cyclosporin A, Lumulse 42-L®, and an oil having a Specific Gravity below 0.9.

Another embodiment comprises cyclosporin A, Lumulse 42-S®, and an oil having a Specific Gravity below 0.9.

Another embodiment comprises cyclosporin A, Lumulse 100-S®, and an oil having a Specific Gravity below 0.9.

Another embodiment comprises cyclosporin A, Lumulse 602-S®, and an oil having a Specific Gravity below 0.9.

Another embodiment comprises cyclosporin A, Magrabar PGE-20-0®, and an oil having a Specific Gravity below 0.9.

Another embodiment comprises cyclosporin A, Magrabar PGE-20L®, and an oil having a Specific Gravity below 0.9.

Another embodiment comprises cyclosporin A, Magrabar PGE-20T®, and an oil having a Specific Gravity below 0.9.

Another embodiment comprises cyclosporin A, Magrabar PGE-22-0®, and an oil having a Specific Gravity below 0.9.

Another embodiment comprises cyclosporin A, Magrabar PGE-22L®, and an oil having a Specific Gravity below 0.9.

Another embodiment comprises cyclosporin A, Magrabar PGE-22T®, and an oil having a Specific Gravity below 0.9.

Another embodiment comprises cyclosporin A, Magrabar PGE-40-0®, and an oil having a Specific Gravity below 0.9.

Another embodiment comprises cyclosporin A, Magrabar PGE-40L®, and an oil having a Specific Gravity below 0.9.

Another embodiment comprises cyclosporin A, Magrabar PGE-40T®, and an oil having a Specific Gravity below 0.9.

Another embodiment comprises cyclosporin A, Magrabar PGE-42-0®, and an oil having a Specific Gravity below 0.9.

Another embodiment comprises cyclosporin A, Magrabar PGE-42L®, and an oil having a Specific Gravity below 0.9.

Another embodiment comprises cyclosporin A, Magrabar PGE-42T®, and an oil having a Specific Gravity below 0.9.

Another embodiment comprises cyclosporin A, Magrabar PGE-60-0®, and an oil having a Specific Gravity below 0.9.

Another embodiment comprises cyclosporin A, Magrabar PGE-60L®, and an oil having a Specific Gravity below 0.9.

Another embodiment comprises cyclosporin A, Magrabar PGE-60T®, and an oil having a Specific Gravity below 0.9.

Another embodiment comprises cyclosporin A, Magrabar PGE-62-0®, and an oil having a Specific Gravity below 0.9.

Another embodiment comprises cyclosporin A, Magrabar PGE-62L®, and an oil having a Specific Gravity below 0.9.

Another embodiment comprises cyclosporin A, Magrabar PGE-62T®, and an oil having a Specific Gravity below 0.9.

Another embodiment comprises cyclosporin A, Mapeg S-40K®, and an oil having a Specific Gravity below 0.9.

Another embodiment comprises cyclosporin A, Marlowet OTS®, and an oil having a Specific Gravity below 0.9.

Another embodiment comprises cyclosporin A, Naturechem PGR®, and an oil having a Specific Gravity below 0.9.

Another embodiment comprises cyclosporin A, PG No. 4®, and an oil having a Specific Gravity below 0.9.

Another embodiment comprises cyclosporin A, Renex 20®, and an oil having a Specific Gravity below 0.9.

Another embodiment comprises cyclosporin A, Ritox 52®, and an oil having a Specific Gravity-below 0.9.

Another embodiment comprises cyclosporin A, Ritox 53®, and an oil having a Specific Gravity below 0.9.

Another embodiment comprises cyclosporin A, Ritox 59®, and an oil having a Specific Gravity below 0.9.

Another embodiment comprises cyclosporin A, Surfax 8916/A®, and an oil having a Specific Gravity below 0.9.

Another embodiment comprises cyclosporin A, Tego Acid S 40 P®, and an oil having a Specific Gravity below 0.9.

Another embodiment comprises cyclosporin A, Tego Acid S 100 P®, and an oil having a Specific Gravity below 0.9.

Another embodiment comprises cyclosporin A, Tween 20®, and an oil having a Specific Gravity below 0.9.

Another embodiment comprises cyclosporin A, Volpo 131®, and an oil having a Specific Gravity below 0.9.

Another embodiment comprises cyclosporin A, an Ethoxylated Fatty Ester or Oil, and an oil having a Specific Gravity below 0.9.

Another embodiment comprises cyclosporin A, Acconon 6-C10®, and an oil having a Specific Gravity below 0.9.

Another embodiment comprises cyclosporin A, Acconon CC-6®, and an oil having a Specific Gravity below 0.9.

Another embodiment comprises cyclosporin A, Acconon CO-7®, and an oil having a Specific Gravity below 0.9.

Another embodiment comprises cyclosporin A, Aldosperse 40/60 FG®, and an oil having a Specific Gravity below 0.9.

Another embodiment comprises cyclosporin A, Aldosperse ML-23®, and an oil having a Specific Gravity below 0.9.

Another embodiment comprises cyclosporin A, Aldosperse MS-20 FG®, and an oil having a Specific Gravity below 0.9.

Another embodiment comprises cyclosporin A, Alkamuls EL-620®, and an oil having a Specific Gravity below 0.9.

Another embodiment comprises cyclosporin A, Alkamuls EL-719®, and an oil having a Specific Gravity below 0.9.

Another embodiment comprises cyclosporin A, Alkamuls EL-985®, and an oil having a Specific Gravity below 0.9.

Another embodiment comprises cyclosporin A, Arlatone G®, and an oil having a Specific Gravity below 0.9.

Another embodiment comprises cyclosporin A, Arlatone T®, and an oil having a Specific Gravity below 0.9.

Another embodiment comprises cyclosporin A, Atlas G-1045A®, and an oil having a Specific Gravity below 0.9.

Another embodiment comprises cyclosporin A, Atlas G-1086®, and an oil having a Specific Gravity below 0.9.

Another embodiment comprises cyclosporin A, Atlas G-1087®, and an oil having a Specific Gravity below 0.9.

Another embodiment comprises cyclosporin A, Atlas G-1089®, and an oil having a Specific Gravity below 0.9.

Another embodiment comprises cyclosporin A, Atlas G-1096®, and an oil having a Specific Gravity below 0.9.

Another embodiment comprises cyclosporin A, Atlas G-1292®, and an oil having a Specific Gravity below 0.9.

Another embodiment comprises cyclosporin A, Atlas G-1293®, and an oil having a Specific Gravity below 0.9.

Another embodiment comprises cyclosporin A, Atlas G-1300®, and an oil having a Specific Gravity below 0.9.

Another embodiment comprises cyclosporin A, Atlas G-7076®, and an oil having a Specific Gravity below 0.9.

Another embodiment comprises cyclosporin A, Capmul EMG®, and an oil having a Specific Gravity below 0.9.

Another embodiment comprises cyclosporin A, Chemonic CO-40®, and an oil having a Specific Gravity below 0.9.

Another embodiment comprises cyclosporin A, Chemonic LI-3®, and an oil having a Specific Gravity below 0.9.

Another embodiment comprises cyclosporin A, Chemonic LI-7®, and an oil having a Specific Gravity below 0.9.

Another embodiment comprises cyclosporin A, Cirrasol GM®, and an oil having a Specific Gravity below 0.9.

Another embodiment comprises cyclosporin A, Cremophor CO 40®, and an oil having a Specific Gravity below 0.9.

Another embodiment comprises cyclosporin A, Cremophor CO 410®, and an oil having a Specific Gravity below 0.9.

Another embodiment comprises cyclosporin A, Cremophor EL®, and an oil having a Specific Gravity below 0.9.

Another embodiment comprises cyclosporin A, Cremophor GC7®, and an oil having a Specific Gravity below 0.9.

Another embodiment comprises cyclosporin A, Cremophor RH-40®, and an oil having a Specific Gravity below 0.9.

Another embodiment comprises cyclosporin A, Crovol A-40®, and an oil having a Specific Gravity below 0.9.

Another embodiment comprises cyclosporin A, Crovol A-70®, and an oil having a Specific Gravity below 0.9.

Another embodiment comprises cyclosporin A, Crovol M-70®, and an oil having a Specific Gravity below 0.9.

Another embodiment comprises cyclosporin A, Crovol PK-70®, and an oil having a Specific Gravity below 0.9.

Another embodiment comprises cyclosporin A, Cutina E-24®, and an oil having a Specific Gravity below 0.9.

Another embodiment comprises cyclosporin A, Dacospin 12-R®, and an oil having a Specific Gravity below 0.9.

Another embodiment comprises cyclosporin A, Dehymuls HRE-7®, and an oil having a Specific Gravity below 0.9.

Another embodiment comprises cyclosporin A, DeSonic 30C®, and an oil having a Specific Gravity below 0.9.

Another embodiment comprises cyclosporin A, DeSonic 36C®, and an oil having a Specific Gravity below 0.9.

Another embodiment comprises cyclosporin A, DeSonic 40C®, and an oil having a Specific Gravity below 0.9.

Another embodiment comprises cyclosporin A, Durfax 60®, and an oil having a Specific Gravity below 0.9.

Another embodiment comprises cyclosporin A, Durfax 65®, and an oil having a Specific Gravity below 0.9.

Another embodiment comprises cyclosporin A, Durfax 80®, and an oil having a Specific Gravity below 0.9.

Another embodiment comprises cyclosporin A, Durfax EOM®, and an oil having a Specific Gravity below 0.9.

Another embodiment comprises cyclosporin A, Eccoterge NF-2®, and an oil having a Specific Gravity below 0.9.

Another embodiment comprises cyclosporin A, Emulpon CO-360®, and an oil having a Specific Gravity below 0.9.

Another embodiment comprises cyclosporin A, Emulpon CO-550®, and an oil having a Specific Gravity below 0.9.

Another embodiment comprises cyclosporin A, Emulsogen EL®, and an oil having a Specific Gravity below 0.9.

Another embodiment comprises cyclosporin A, Emulsogen HCO 040®, and an oil having a Specific Gravity below 0.9.

Another embodiment comprises cyclosporin A, Emulsogen HCO 060®, and an oil having a Specific Gravity below 0.9.

Another embodiment comprises cyclosporin A, Emulsynt 1055®, and an oil having a Specific Gravity below 0.9.

Another embodiment comprises cyclosporin A, Ethox 3095®, and an oil having a Specific Gravity below 0.9.

Another embodiment comprises cyclosporin A, Eumulgin RO-40®, and an oil having a Specific Gravity below 0.9.

Another embodiment comprises cyclosporin A, Genapol G-260®, and an oil having a Specific Gravity below 0.9.

Another embodiment comprises cyclosporin A, Glycosperse L-20®, and an oil having a Specific Gravity below 0.9.

Another embodiment comprises cyclosporin A, Glycosperse O-5®, and an oil having a Specific Gravity below 0.9.

Another embodiment comprises cyclosporin A, Glycosperse O-20®, and an oil having a Specific Gravity below 0.9.

Another embodiment comprises cyclosporin A, Glycosperse O-20 FG®, and an oil having a Specific Gravity below 0.9.

Another embodiment comprises cyclosporin A, Glycosperse S-20®, and an oil having a Specific Gravity below 0.9.

Another embodiment comprises cyclosporin A, Glycosperse S-20 FG®, and an oil having a Specific Gravity below 0.9.

Another embodiment comprises cyclosporin A, Glycosperse TS-20®, and an oil having a Specific Gravity below 0.9.

Another embodiment comprises cyclosporin A, Glycosperse TS-20 FG®, and an oil having a Specific Gravity below 0.9.

Another embodiment comprises cyclosporin A, Hetan SL®, and an oil having a Specific Gravity below 0.9.

Another embodiment comprises cyclosporin A, Hetan SO®, and an oil having a Specific Gravity below 0.9.

Another embodiment comprises cyclosporin A, Hetan SS®, and an oil having a Specific Gravity below 0.9.

Another embodiment comprises cyclosporin A, Hetoxide C-2®, and an oil having a Specific Gravity below 0.9.

Another embodiment comprises cyclosporin A, Hetoxide C-9®, and an oil having a Specific Gravity below 0.9.

Another embodiment comprises cyclosporin A, Hetoxide C-15®, and an oil having a Specific Gravity below 0.9.

Another embodiment comprises cyclosporin A, Hetoxide C-25®, and an oil having a Specific Gravity below 0.9.

Another embodiment comprises cyclosporin A, Hetoxide C-40®, and an oil having a Specific Gravity below 0.9.

Another embodiment comprises cyclosporin A, Hetoxide C-200®, and an oil having a Specific Gravity below 0.9.

Another embodiment comprises cyclosporin A, Hetoxide C-200-50%®, and an oil having a Specific Gravity below 0.9.

Another embodiment comprises cyclosporin A, Hetoxide GC-30®, and an oil having a Specific Gravity below 0.9.

Another embodiment comprises cyclosporin A, Hetoxide HC-60®, and an oil having a Specific Gravity below 0.9.

Another embodiment comprises cyclosporin A, Ice No. 2®, and an oil having a Specific Gravity below 0.9.

Another embodiment comprises cyclosporin A, Incrocas 30/40®, and an oil having a Specific Gravity below 0.9.

Another embodiment comprises cyclosporin A, Lexol EC®, and an oil having a Specific Gravity below 0.9.

Another embodiment comprises cyclosporin A, Lexol EC®, and an oil having a Specific Gravity below 0.9.

Another embodiment comprises cyclosporin A, Lipocol HCO-40®, and an oil having a Specific Gravity below 0.9.

Another embodiment comprises cyclosporin A, Lipocol HCO-60®, and an oil having a Specific Gravity below 0.9.

Another embodiment comprises cyclosporin A, Lipocol O-3 Special®, and an oil having a Specific Gravity below 0.9.

Another embodiment comprises cyclosporin A, Lipopeg 2-L®, and an oil having a Specific Gravity below 0.9.

Another embodiment comprises cyclosporin A, Lipopeg 4-DO®, and an oil having a Specific Gravity below 0.9.

Another embodiment comprises cyclosporin A, Lipopeg 4-DS®, and an oil having a Specific Gravity below 0.9.

Another embodiment comprises cyclosporin A, Lipovol GTB®, and an oil having a Specific Gravity below 0.9.

Another embodiment comprises cyclosporin A, Lonzest SML-20®, and an oil having a Specific Gravity below 0.9.

Another embodiment comprises cyclosporin A, Lonzest SMO-20®, and an oil having a Specific Gravity below 0.9.

Another embodiment comprises cyclosporin A, Lonzest SMS-20®, and an oil having a Specific Gravity below 0.9.

Another embodiment comprises cyclosporin A, Lonzest STO-20®, and an oil having a Specific Gravity below 0.9.

Another embodiment comprises cyclosporin A, Lonzest STS-20®, and an oil having a Specific Gravity below 0.9.

Another embodiment comprises cyclosporin A, Lumulse GR-40®, and an oil having a Specific Gravity below 0.9.

Another embodiment comprises cyclosporin A, Lumulse GRH-40®, and an oil having a Specific Gravity below 0.9.

Another embodiment comprises cyclosporin A, Lumulse POE (7) GML®, and an oil having a Specific Gravity below 0.9.

Another embodiment comprises cyclosporin A, Lumulse POE (12) Glyc®, and an oil having a Specific Gravity below 0.9.

Another embodiment comprises cyclosporin A, Lumulse POE (40) MS KP®, and an oil having a Specific Gravity below 0.9.

Another embodiment comprises cyclosporin A, Marlowet 4750®, and an oil having a Specific Gravity below 0.9.

Another embodiment comprises cyclosporin A, Marlowet LVS®, and an oil having a Specific Gravity below 0.9.

Another embodiment comprises cyclosporin A, Marlowet R 11®, and an oil having a Specific Gravity below 0.9.

Another embodiment comprises cyclosporin A, Marlowet R 40®, and an oil having a Specific Gravity below 0.9.

Another embodiment comprises cyclosporin A, Mazol 80 MGK®, and an oil having a Specific Gravity below 0.9.

Another embodiment comprises cyclosporin A, Nonionic Emulsifier T-9®, and an oil having a Specific Gravity below 0.9.

Another embodiment comprises cyclosporin A, Oronal LCG®, and an oil having a Specific Gravity below 0.9.

Another embodiment comprises cyclosporin A, Polyderm PPI-CO-200®, and an oil having a Specific Gravity below 0.9.

Another embodiment comprises cyclosporin A, Polyderm PPI-CO-40®, and an oil having a Specific Gravity below 0.9.

Another embodiment comprises cyclosporin A, Rewoderm LI 520-70®, and an oil having a Specific Gravity below 0.9.

Another embodiment comprises cyclosporin A, Ritapeg 150 DS®, and an oil having a Specific Gravity below 0.9.

Another embodiment comprises cyclosporin A, Softigen 767®, and an oil having a Specific Gravity below 0.9.

Another embodiment comprises cyclosporin A, Surfactol 318®, and an oil having a Specific Gravity below 0.9.

Another embodiment comprises cyclosporin A, Surfactol 365®, and an oil having a Specific Gravity below 0.9.

Another embodiment comprises cyclosporin A, Syn Lube 107®, and an oil having a Specific Gravity below 0.9.

Another embodiment comprises cyclosporin A, Syn Lube 728®, and an oil having a Specific Gravity below 0.9.

Another embodiment comprises cyclosporin A, Syn Lube 1632H®, and an oil having a Specific Gravity below 0.9.

Another embodiment comprises cyclosporin A, Syn Lube 6277-A®, and an oil having a Specific Gravity below 0.9.

Another embodiment comprises cyclosporin A, T-Det C-20®, and an oil having a Specific Gravity below 0.9.

Another embodiment comprises cyclosporin A, T-Det C-40®, and an oil having a Specific Gravity below 0.9.

Another embodiment comprises cyclosporin A, Tally 100 Plus®, and an oil having a Specific Gravity below 0.9.

Another embodiment comprises cyclosporin A, Uniperol EL®, and an oil having a Specific Gravity below 0.9.

Another embodiment comprises cyclosporin A, a Fatty Ester, and an oil having a Specific Gravity below 0.9.

Another embodiment comprises cyclosporin A, Actralube-Syn 147®, and an oil having a Specific Gravity below 0.9.

Another embodiment comprises cyclosporin A, Atlas G-1556®, and an oil having a Specific Gravity below 0.9.

Another embodiment comprises cyclosporin A, Atlas G-1564®, and an oil having a Specific Gravity below 0.9.

Another embodiment comprises cyclosporin A, Atlasol Base Oil S®, and an oil having a Specific Gravity below 0.9.

Another embodiment comprises cyclosporin A, Base ML®, and an oil having a Specific Gravity below 0.9.

Another embodiment comprises cyclosporin A, Base MT®, and an oil having a Specific Gravity below 0.9.

Another embodiment comprises cyclosporin A, Cerasynt 303®, and an oil having a Specific Gravity below 0.9.

Another embodiment comprises cyclosporin A, Dermol 1012®, and an oil having a Specific Gravity below 0.9.

Another embodiment comprises cyclosporin A, Kemester 4000®, and an oil having a Specific Gravity below 0.9.

Another embodiment comprises cyclosporin A, Lactipol S®, and an oil having a Specific Gravity below 0.9.

Another embodiment comprises cyclosporin A, Magrabar PGO®, and an oil having a Specific Gravity below 0.9.

Another embodiment comprises cyclosporin A, Mayco Base BFO®, and an oil having a Specific Gravity below 0.9.

Another embodiment comprises cyclosporin A, Methyl Linoleate®, and an oil having a Specific Gravity below 0.9.

Another embodiment comprises cyclosporin A, Pationic 122A®, and an oil having a Specific Gravity below 0.9.

Another embodiment comprises cyclosporin A, Pationic 138C®, and an oil having a Specific Gravity below 0.9.

Another embodiment comprises cyclosporin A, Pationic CSL®, and an oil having a Specific Gravity below 0.9.

Another embodiment comprises cyclosporin A, Pationic ISL®, and an oil having a Specific Gravity below 0.9.

Another embodiment comprises cyclosporin A, Pationic SBL®, and an oil having a Specific Gravity below 0.9.

Another embodiment comprises cyclosporin A, Pationic SSL®, and an oil having a Specific Gravity below 0.9.

Another embodiment comprises cyclosporin A, Ritasol®, and an oil having a Specific Gravity below 0.9.

Another embodiment comprises cyclosporin A, Tego Alkanol CS 20®, and an oil having a Specific Gravity below 0.9.

Another embodiment comprises cyclosporin A, Tego Alkanol L23 P®, and an oil having a Specific Gravity below 0.9.

Another embodiment comprises cyclosporin A, Tego Alkanol S2®, and an oil having a Specific Gravity below 0.9.

Another embodiment comprises cyclosporin A, Tego Alkanol S20 P®, and an oil having a Specific Gravity below 0.9.

Another embodiment comprises cyclosporin A, Triemulsifier 600 MS®, and an oil having a Specific Gravity below 0.9.

Another embodiment comprises cyclosporin A, a Glycerol Ester, and an oil having a Specific Gravity below 0.9.

Another embodiment comprises cyclosporin A, Agro #9 Wint SBO®, and an oil having a Specific Gravity below 0.9.

Another embodiment comprises cyclosporin A, Ahcovel Base 700®, and an oil having a Specific Gravity below 0.9.

Another embodiment comprises cyclosporin A, Aldo HMS FG®, and an oil having a Specific Gravity below 0.9.

Another embodiment comprises cyclosporin A, Aldo MLD®, and an oil having a Specific Gravity below 0.9.

Another embodiment comprises cyclosporin A, Aldo MLD FG®, and an oil having a Specific Gravity below 0.9.

Another embodiment comprises cyclosporin A, Aldo MO FG®, and an oil having a Specific Gravity below 0.9.

Another embodiment comprises cyclosporin A, Aldo MS®, and an oil having a Specific Gravity below 0.9.

Another embodiment comprises cyclosporin A, Aldo MS FG®, and an oil having a Specific Gravity below 0.9.

Another embodiment comprises cyclosporin A, Aldo MS LG FG®, and an oil having a Specific Gravity below 0.9.

Another embodiment comprises cyclosporin A, Aldo MSD®, and an oil having a Specific Gravity below 0.9.

Another embodiment comprises cyclosporin A, Aldo MSD FG®, and an oil having a Specific Gravity below 0.9.

Another embodiment comprises cyclosporin A, Aldosperse O-20 FG®, and an oil having a Specific Gravity below 0.9.

Another embodiment comprises cyclosporin A, Aldosperse TS-20 FG®, and an oil having a Specific Gravity below 0.9.

Another embodiment comprises cyclosporin A, Aldosperse TS-40 FG®, and an oil having a Specific Gravity below 0.9.

Another embodiment comprises cyclosporin A, Arlacel 165®, and an oil having a Specific Gravity below 0.9.

Another embodiment comprises cyclosporin A, Arlacel 186®, and an oil having a Specific Gravity below 0.9.

Another embodiment comprises cyclosporin A, Capmul GMO®, and an oil having a Specific Gravity below 0.9.

Another embodiment comprises cyclosporin A, Capmul GMS®, and an oil having a Specific Gravity below 0.9.

Another embodiment comprises cyclosporin A, Caprol 3GO®, and an oil having a Specific Gravity below 0.9.

Another embodiment comprises cyclosporin A, Caprol 3GVS®, and an oil having a Specific Gravity below 0.9.

Another embodiment comprises cyclosporin A, Caprol 6G2S®, and an oil having a Specific Gravity below 0.9.

Another embodiment comprises cyclosporin A, Caprol 10G40®, and an oil having a Specific Gravity below 0.9.

Another embodiment comprises cyclosporin A, Caprol 10G100®, and an oil having a Specific Gravity below 0.9.

Another embodiment comprises cyclosporin A, Caprol ET®, and an oil having a Specific Gravity below 0.9.

Another embodiment comprises cyclosporin A, Caprol PGE860®, and an oil having a Specific Gravity below 0.9.

Another embodiment comprises cyclosporin A, Cerasynt 945®, and an oil having a Specific Gravity below 0.9.

Another embodiment comprises cyclosporin A, Cerasynt GMS®, and an oil having a Specific Gravity below 0.9.

Another embodiment comprises cyclosporin A, Cerasynt Q®, and an oil having a Specific Gravity below 0.9.

Another embodiment comprises cyclosporin A, Cerasynt SD®, and an oil having a Specific Gravity below 0.9.

Another embodiment comprises cyclosporin A, Cerasynt WM®, and an oil having a Specific Gravity below 0.9.

Another embodiment comprises cyclosporin A, Chemsperse 14®, and an oil having a Specific Gravity below 0.9.

Another embodiment comprises cyclosporin A, Cremophor GO-32®, and an oil having a Specific Gravity below 0.9.

Another embodiment comprises cyclosporin A, Cremophor GS11®, and an oil having a Specific Gravity below 0.9.

Another embodiment comprises cyclosporin A, Cremophor GS-32®, and an oil having a Specific Gravity below 0.9.

Another embodiment comprises cyclosporin A, Cutina KD-16®, and an oil having a Specific Gravity below 0.9.

Another embodiment comprises cyclosporin A, Dehymuls PGPH®, and an oil having a Specific Gravity below 0.9.

Another embodiment comprises cyclosporin A, Dermol DGDIS®, and an oil having a Specific Gravity below 0.9.

Another embodiment comprises cyclosporin A, Dermol DGMIS®, and an oil having a Specific Gravity below 0.9.

Another embodiment comprises cyclosporin A, Dermol G-76®, and an oil having a Specific Gravity below 0.9.

Another embodiment comprises cyclosporin A, Dermol G-7DI®, and an oil having a Specific Gravity below 0.9.

Another embodiment comprises cyclosporin A, Dermol NGDI®, and an oil having a Specific Gravity below 0.9.

Another embodiment comprises cyclosporin A, Dermolan GLH®, and an oil having a Specific Gravity below 0.9.

Another embodiment comprises cyclosporin A, Drewmulse GMO®, and an oil having a Specific Gravity below 0.9.

Another embodiment comprises cyclosporin A, Drewpol 3-5-M®, and an oil having a Specific Gravity below 0.9.

Another embodiment comprises cyclosporin A, Durlac 100 W®, and an oil having a Specific Gravity below 0.9.

Another embodiment comprises cyclosporin A, Dur-Lo®, and an oil having a Specific Gravity below 0.9.

Another embodiment comprises cyclosporin A, Dynasan 118®, and an oil having a Specific Gravity below 0.9.

Another embodiment comprises cyclosporin A, EC-25®, and an oil having a Specific Gravity below 0.9.

Another embodiment comprises cyclosporin A, EM 40®, and an oil having a Specific Gravity below 0.9.

Another embodiment comprises cyclosporin A, Emerest 2400®, and an oil having a Specific Gravity below 0.9.

Another embodiment comprises cyclosporin A, Emerest 2452®, and an oil having a Specific Gravity below 0.9.

Another embodiment comprises cyclosporin A, Empilan G-26®, and an oil having a Specific Gravity below 0.9.

Another embodiment comprises cyclosporin A, Genapol TSM®, and an oil having a Specific Gravity below 0.9.

Another embodiment comprises cyclosporin A, Hostacerin DGI®, and an oil having a Specific Gravity below 0.9.

Another embodiment comprises cyclosporin A, Hostacerin DGL®, and an oil having a Specific Gravity below 0.9.

Another embodiment comprises cyclosporin A, Hostacerin DGMS®, and an oil having a Specific Gravity below 0.9.

Another embodiment comprises cyclosporin A, Hostacerin DGSB®, and an oil having a Specific Gravity below 0.9.

Another embodiment comprises cyclosporin A, Ice No. 2®, and an oil having a Specific Gravity below 0.9.

Another embodiment comprises cyclosporin A, Imwitor 742®, and an oil having a Specific Gravity below 0.9.

Another embodiment comprises cyclosporin A, Imwitor 780 K®, and an oil having a Specific Gravity below 0.9.

Another embodiment comprises cyclosporin A, Imwitor 960 Flakes®, and an oil having a Specific Gravity below 0.9.

Another embodiment comprises cyclosporin A, Isolan GI 34®, and an oil having a Specific Gravity below 0.9.

Another embodiment comprises cyclosporin A, Isolan GO 33®, and an oil having a Specific Gravity below 0.9.

Another embodiment comprises cyclosporin A, Kemester 1000®, and an oil having a Specific Gravity below 0.9.

Another embodiment comprises cyclosporin A, Kemester 2000®, and an oil having a Specific Gravity below 0.9.

Another embodiment comprises cyclosporin A, Kemester 2000®, and an oil having a Specific Gravity below 0.9.

Another embodiment comprises cyclosporin A, Kemester 6000SE®, and an oil having a Specific Gravity below 0.9.

Another embodiment comprises cyclosporin A, Lamecreme DGE 18®, and an oil having a Specific Gravity below 0.9.

Another embodiment comprises cyclosporin A, Lexemul 515®, and an oil having a Specific Gravity below 0.9.

Another embodiment comprises cyclosporin A, Lexemul 561®, and an oil having a Specific Gravity below 0.9.

Another embodiment comprises cyclosporin A, Lexemul AR®, and an oil having a Specific Gravity below 0.9.

Another embodiment comprises cyclosporin A, Lexemul AS®, and an oil having a Specific Gravity below 0.9.

Another embodiment comprises cyclosporin A, Lexemul GDL®, and an oil having a Specific Gravity below 0.9.

Another embodiment comprises cyclosporin A, Lexemul T®, and an oil having a Specific Gravity below 0.9.

Another embodiment comprises cyclosporin A, Lipomulse 165®, and an oil having a Specific Gravity below 0.9.

Another embodiment comprises cyclosporin A, Lumulse GML K®, and an oil having a Specific Gravity below 0.9.

Another embodiment comprises cyclosporin A, Lumulse GMO K®, and an oil having a Specific Gravity below 0.9.

Another embodiment comprises cyclosporin A, Lumulse GMR K®, and an oil having a Specific Gravity below 0.9.

Another embodiment comprises cyclosporin A, Lumulse GMT K®, and an oil having a Specific Gravity below 0.9.

Another embodiment comprises cyclosporin A, Magrabar GMC®, and an oil having a Specific Gravity below 0.9.

Another embodiment comprises cyclosporin A, Magrabar GMO-CK®, and an oil having a Specific Gravity below 0.9.

Another embodiment comprises cyclosporin A, Magrabar MDG-5050®, and an oil having a Specific Gravity below 0.9.

Another embodiment comprises cyclosporin A, Magrabar PGO-315®, and an oil having a Specific Gravity below 0.9.

Another embodiment comprises cyclosporin A, Magrabar PGO-1010®, and an oil having a Specific Gravity below 0.9.

Another embodiment comprises cyclosporin A, Mazol 300K®, and an oil having a Specific Gravity below 0.9.

Another embodiment comprises cyclosporin A, Mazol GMO-K®, and an oil having a Specific Gravity below 0.9.

Another embodiment comprises cyclosporin A, Mazol GMS-K®, and an oil having a Specific Gravity below 0.9.

Another embodiment comprises cyclosporin A, Mazol PG031-K®, and an oil having a Specific Gravity below 0.9.

Another embodiment comprises cyclosporin A, Miglyol 812®, and an oil having a Specific Gravity below 0.9.

Another embodiment comprises cyclosporin A, Norfox 165C®, and an oil having a Specific Gravity below 0.9.

Another embodiment comprises cyclosporin A, Schercemol GMIS®, and an oil having a Specific Gravity below 0.9.

Another embodiment comprises cyclosporin A, Tegin®, and an oil having a Specific Gravity below 0.9.

Another embodiment comprises cyclosporin A, Tegin 4100 Pellets®, and an oil having a Specific Gravity below 0.9.

Another embodiment comprises cyclosporin A, Tegin M Pellets®, and an oil having a Specific Gravity below 0.9.

Another embodiment comprises cyclosporin A, Tegin OV®, and an oil having a Specific Gravity below 0.9.

Another embodiment comprises cyclosporin A, Teginacid H®, and an oil having a Specific Gravity below 0.9.

Another embodiment comprises cyclosporin A, Tego Cosmo P813®, and an oil having a Specific Gravity below 0.9.

Another embodiment comprises cyclosporin A, Wickenol 535®, and an oil having a Specific Gravity below 0.9.

Another embodiment comprises cyclosporin A, Witconol 14®, and an oil having a Specific Gravity below 0.9.

Another embodiment comprises cyclosporin A, Witconol 14®, and an oil having a Specific Gravity below 0.9.

Another embodiment comprises cyclosporin A, Witconol 14F®, and an oil having a Specific Gravity below 0.9.

Another embodiment comprises cyclosporin A, Witconol 18L®, and an oil having a Specific Gravity below 0.9.

Another embodiment comprises cyclosporin A, Witconol GOT®, and an oil having a Specific Gravity below 0.9.

Another embodiment comprises cyclosporin A, Witconol MST®, and an oil having a Specific Gravity below 0.9.

Another embodiment comprises cyclosporin A, Witconol RHT®, and an oil having a Specific Gravity below 0.9.

Another embodiment comprises cyclosporin A, a Glycol Ester, and an oil having a Specific Gravity below 0.9.

Another embodiment comprises cyclosporin A, Alkamuls 600 DO®, and an oil having a Specific Gravity below 0.9.

Another embodiment comprises cyclosporin A, Alkamuls SEG®, and an oil having a Specific Gravity below 0.9.

Another embodiment comprises cyclosporin A, Atlas EM-2®, and an oil having a Specific Gravity below 0.9.

Another embodiment comprises cyclosporin A, Cerasynt IP®, and an oil having a Specific Gravity below 0.9.

Another embodiment comprises cyclosporin A, Cerasynt M®, and an oil having a Specific Gravity below 0.9.

Another embodiment comprises cyclosporin A, Cerasynt MN®, and an oil having a Specific Gravity below 0.9.

Another embodiment comprises cyclosporin A, Cerasynt PA®, and an oil having a Specific Gravity below 0.9.

Another embodiment comprises cyclosporin A, Chemsperse EGDS®, and an oil having a Specific Gravity below 0.9.

Another embodiment comprises cyclosporin A, Chemsperse EGMS®, and an oil having a Specific Gravity below 0.9.

Another embodiment comprises cyclosporin A, Colonial Monolaurin®, and an oil having a Specific Gravity below 0.9.

Another embodiment comprises cyclosporin A, DeMuls SGE-95®, and an oil having a Specific Gravity below 0.9.

Another embodiment comprises cyclosporin A, Eccoterge 200®, and an oil having a Specific Gravity below 0.9.

Another embodiment comprises cyclosporin A, Emerest 2380®, and an oil having a Specific Gravity below 0.9.

Another embodiment comprises cyclosporin A, Ethox 2610®, and an oil having a Specific Gravity below 0.9.

Another embodiment comprises cyclosporin A, Ethox DO-9®, and an oil having a Specific Gravity below 0.9.

Another embodiment comprises cyclosporin A, Ethox DO-14®, and an oil having a Specific Gravity below 0.9.

Another embodiment comprises cyclosporin A, Ethox SO-9®, and an oil having a Specific Gravity below 0.9.

Another embodiment comprises cyclosporin A, Fizul MD-318®, and an oil having a Specific Gravity below 0.9.

Another embodiment comprises cyclosporin A, Genapol EGDS-VHP®, and an oil having a Specific Gravity below 0.9.

Another embodiment comprises cyclosporin A, Genapol TS Powder®, and an oil having a Specific Gravity below 0.9.

Another embodiment comprises cyclosporin A, Hostacerin WO®, and an oil having a Specific Gravity below 0.9.

Another embodiment comprises cyclosporin A, Inversol 140®, and an oil having a Specific Gravity below 0.9.

Another embodiment comprises cyclosporin A, Kemester 104®, and an oil having a Specific Gravity below 0.9.

Another embodiment comprises cyclosporin A, Kemester 205®, and an oil having a Specific Gravity below 0.9.

Another embodiment comprises cyclosporin A, Kemester 226®, and an oil having a Specific Gravity below 0.9.

Another embodiment comprises cyclosporin A, Kemester 5221 SE®, and an oil having a Specific Gravity below 0.9.

Another embodiment comprises cyclosporin A, Kemester EGDS®, and an oil having a Specific Gravity below 0.9.

Another embodiment comprises cyclosporin A, Lexemul EGDS®, and an oil having a Specific Gravity below 0.9.

Another embodiment comprises cyclosporin A, Lexemul EGMS®, and an oil having a Specific Gravity below 0.9.

Another embodiment comprises cyclosporin A, Lexemul P®, and an oil having a Specific Gravity below 0.9.

Another embodiment comprises cyclosporin A, Lipo DGLS®, and an oil having a Specific Gravity below 0.9.

Another embodiment comprises cyclosporin A, Lipo EGDS®, and an oil having a Specific Gravity below 0.9.

Another embodiment comprises cyclosporin A, Lipo PGMS®, and an oil having a Specific Gravity below 0.9.

Another embodiment comprises cyclosporin A, Liposorb S-4®, and an oil having a Specific Gravity below 0.9.

Another embodiment comprises cyclosporin A, Liposorb TO-20®, and an oil having a Specific Gravity below 0.9.

Another embodiment comprises cyclosporin A, Lumulse PGO®, and an oil having a Specific Gravity below 0.9.

Another embodiment comprises cyclosporin A, Mackester EGDS®, and an oil having a Specific Gravity below 0.9.

Another embodiment comprises cyclosporin A, Mackester EGMS®, and an oil having a Specific Gravity below 0.9.

Another embodiment comprises cyclosporin A, Mackester GSTP®, and an oil having a Specific Gravity below 0.9.

Another embodiment comprises cyclosporin A, Mackester Series®, and an oil having a Specific Gravity below 0.9.

Another embodiment comprises cyclosporin A, Magrabar PDG-50®, and an oil having a Specific Gravity below 0.9.

Another embodiment comprises cyclosporin A, Mapeg 6000 DS®, and an oil having a Specific Gravity below 0.9.

Another embodiment comprises cyclosporin A, Marlowet 4702®, and an oil having a Specific Gravity below 0.9.

Another embodiment comprises cyclosporin A, Monalube 305®, and an oil having a Specific Gravity below 0.9.

Another embodiment comprises cyclosporin A, Monalube 310®, and an oil having a Specific Gravity below 0.9.

Another embodiment comprises cyclosporin A, Monalube 315®, and an oil having a Specific Gravity below 0.9.

Another embodiment comprises cyclosporin A, Monalube 320®, and an oil having a Specific Gravity below 0.9.

Another embodiment comprises cyclosporin A, Monalube 325®, and an oil having a Specific Gravity below 0.9.

Another embodiment comprises cyclosporin A, Monalube 330®, and an oil having a Specific Gravity below 0.9.

Another embodiment comprises cyclosporin A, Naturechem PGHS®, and an oil having a Specific Gravity below 0.9.

Another embodiment comprises cyclosporin A, Polycastorol PLO-840®, and an oil having a Specific Gravity below 0.9.

Another embodiment comprises cyclosporin A, Polytex 10M®, and an oil having a Specific Gravity below 0.9.

Another embodiment comprises cyclosporin A, Ritasynt IP®, and an oil having a Specific Gravity below 0.9.

Another embodiment comprises cyclosporin A, Ross Chem PEG 600 DT®, and an oil having a Specific Gravity below 0.9.

Another embodiment comprises cyclosporin A, Schercemol PGMS®, and an oil having a Specific Gravity below 0.9.

Another embodiment comprises cyclosporin A, Sponto H-44C®, and an oil having a Specific Gravity below 0.9.

Another embodiment comprises cyclosporin A, Tegin G®, and an oil having a Specific Gravity below 0.9.

Another embodiment comprises cyclosporin A, Witbreak DGE-182®, and an oil having a Specific Gravity below 0.9.

Another embodiment comprises cyclosporin A, Witbreak DGE-182®, and an oil having a Specific Gravity below 0.9.

Another embodiment comprises cyclosporin A, Witbreak DRA-21®, and an oil having a Specific Gravity below 0.9.

Another embodiment comprises cyclosporin A, Witbreak DRA-21®, and an oil having a Specific Gravity below 0.9.

Another embodiment comprises cyclosporin A, Witbreak DRA-50®, and an oil having a Specific Gravity below 0.9.

Another embodiment comprises cyclosporin A, Witbreak DRA-50®, and an oil having a Specific Gravity below 0.9.

Another embodiment comprises cyclosporin A, Witconol F26-46®, and an oil having a Specific Gravity below 0.9.

Another embodiment comprises cyclosporin A, Witconol H-32®, and an oil having a Specific Gravity below 0.9.

Another embodiment comprises cyclosporin A, Witconol H-33®, and an oil having a Specific Gravity below 0.9.

Another embodiment comprises cyclosporin A, Witconol H-35A®, and an oil having a Specific Gravity below 0.9.

Another embodiment comprises cyclosporin A, Witconol RHP®, and an oil having a Specific Gravity below 0.9.

Another embodiment comprises cyclosporin A, a Lanolin-based Derivative, and an oil having a Specific Gravity below 0.9.

Another embodiment comprises cyclosporin A, Amerchol CAB®, and an oil having a Specific Gravity below 0.9.

Another embodiment comprises cyclosporin A, Amerchol L-101®, and an oil having a Specific Gravity below 0.9.

Another embodiment comprises cyclosporin A, Amerlate LFA-LO®, and an oil having a Specific Gravity below 0.9.

Another embodiment comprises cyclosporin A, Amerlate P®, and an oil having a Specific Gravity below 0.9.

Another embodiment comprises cyclosporin A, Barre Common Degras®, and an oil having a Specific Gravity below 0.9.

Another embodiment comprises cyclosporin A, Cholesterol NF®, and an oil having a Specific Gravity below 0.9.

Another embodiment comprises cyclosporin A, Crodalan AWS®, and an oil having a Specific Gravity below 0.9.

Another embodiment comprises cyclosporin A, Crodalan LA®, and an oil having a Specific Gravity below 0.9.

Another embodiment comprises cyclosporin A, Emery 1650®, and an oil having a Specific Gravity below 0.9.

Another embodiment comprises cyclosporin A, Emery 1650®, and an oil having a Specific Gravity below 0.9.

Another embodiment comprises cyclosporin A, Emery 1740®, and an oil having a Specific Gravity below 0.9.

Another embodiment comprises cyclosporin A, Emery 1740®, and an oil having a Specific Gravity below 0.9.

Another embodiment comprises cyclosporin A, Forlan 500®, and an oil having a Specific Gravity below 0.9.

Another embodiment comprises cyclosporin A, Forlan L®, and an oil having a Specific Gravity below 0.9.

Another embodiment comprises cyclosporin A, Laneto 50®, and an oil having a Specific Gravity below 0.9;

Another embodiment comprises cyclosporin A, Laneto 100®, and an oil having a Specific Gravity below 0.9.

Another embodiment comprises cyclosporin A, Laneto AWS®, and an oil having a Specific Gravity below 0.9.

Another embodiment comprises cyclosporin A, Lanfrax 1776®, and an oil having a Specific Gravity below 0.9.

Another embodiment comprises cyclosporin A, Lanfrax 1776®, and an oil having a Specific Gravity below 0.9.

Another embodiment comprises cyclosporin A, Lanogel 21®, and an oil having a Specific Gravity below 0.9.

Another embodiment comprises cyclosporin A, Lipolan®, and an oil having a Specific Gravity below 0.9.

Another embodiment comprises cyclosporin A, Lipolan 31®, and an oil having a Specific Gravity below 0.9.

Another embodiment comprises cyclosporin A, OHlan®, and an oil having a Specific Gravity below 0.9.

Another embodiment comprises cyclosporin A, Polychol 5®, and an oil having a Specific Gravity below 0.9.

Another embodiment comprises cyclosporin A, Polychol 15®, and an oil having a Specific Gravity below 0.9.

Another embodiment comprises cyclosporin A, Ritacetyl®, and an oil having a Specific Gravity below 0.9.

Another embodiment comprises cyclosporin A, Ritachol®, and an oil having a Specific Gravity below 0.9.

Another embodiment comprises cyclosporin A, Ritahydrox®, and an oil having a Specific Gravity below 0.9.

Another embodiment comprises cyclosporin A, Ritalafa®, and an oil having a Specific Gravity below 0.9.

Another embodiment comprises cyclosporin A, Ritalan®, and an oil having a Specific Gravity below 0.9.

Another embodiment comprises cyclosporin A, Ritalan AWS®, and an oil having a Specific Gravity below 0.9.

Another embodiment comprises cyclosporin A, Ritalan C®, and an oil having a Specific Gravity below 0.9.

Another embodiment comprises cyclosporin A, Ritawax®, and an oil having a Specific Gravity below 0.9.

Another embodiment comprises cyclosporin A, Ritawax AEO®, and an oil having a Specific Gravity below 0.9.

Another embodiment comprises cyclosporin A, Ritawax ALA®, and an oil having a Specific Gravity below 0.9.

Another embodiment comprises cyclosporin A, Solan/Solan 50/Super Solan®, and an oil having a Specific Gravity below 0.9.

Another embodiment comprises cyclosporin A, Super Hartolan/Hartolan®, and an oil having a Specific Gravity below 0.9.

Another embodiment comprises cyclosporin A, Supersat AWS-4®, and an oil having a Specific Gravity below 0.9.

Another embodiment comprises cyclosporin A, Supersat AWS-24®, and an oil having a Specific Gravity below 0.9.

Another embodiment comprises cyclosporin A, a Lecithin or Lecithin Derivative, and an oil having a Specific Gravity below 0.9.

Another embodiment comprises cyclosporin A, Alcolec®, and an oil having a Specific Gravity below 0.9.

Another embodiment comprises cyclosporin A, Lecithin®, and an oil having a Specific Gravity below 0.9.

Another embodiment comprises cyclosporin A, Lexin K®, and an oil having a Specific Gravity below 0.9.

Another embodiment comprises cyclosporin A, Natipide®, and an oil having a Specific Gravity below 0.9.

Another embodiment comprises cyclosporin A, a Lignin or Lignin Derivative, and an oil having a Specific Gravity below 0.9.

Another embodiment comprises cyclosporin A, Diwatex XP 9®, and an oil having a Specific Gravity below 0.9.

Another embodiment comprises cyclosporin A, Dynasperse LCD®, and an oil having a Specific Gravity below 0.9.

Another embodiment comprises cyclosporin A, Indulin SAL®, and an oil having a Specific Gravity below 0.9.

Another embodiment comprises cyclosporin A, Indulin W-1®, and an oil having a Specific Gravity below 0.9.

Another embodiment comprises cyclosporin A, Indulin W-5®, and an oil having a Specific Gravity below 0.9.

Another embodiment comprises cyclosporin A, Lignosol FTA®, and an oil having a Specific Gravity below 0.9.

Another embodiment comprises cyclosporin A, Lignosol SFX-65®, and an oil having a Specific Gravity below 0.9.

Another embodiment comprises cyclosporin A, Marasperse 52 CP®, and an oil having a Specific Gravity below 0.9.

Another embodiment comprises cyclosporin A, Marasperse AG®, and an oil having a Specific Gravity below 0.9.

Another embodiment comprises cyclosporin A, Marasperse CBOS-4®, and an oil having a Specific Gravity below 0.9.

Another embodiment comprises cyclosporin A, Ufoxane 2®, and an oil having a Specific Gravity below 0.9.

Another embodiment comprises cyclosporin A, a Methyl Ester, and an oil having a Specific Gravity below 0.9.

Another embodiment comprises cyclosporin A, E.B. Cleaner AK®, and an oil having a Specific Gravity below 0.9.

Another embodiment comprises cyclosporin A, Oleocal ME-70®, and an oil having a Specific Gravity below 0.9.

Another embodiment comprises cyclosporin A, Trade Name Company®, and an oil having a Specific Gravity below 0.9.

Another embodiment comprises cyclosporin A, Methyl Esters (cont'd)®, and an oil having a Specific Gravity below 0.9.

Another embodiment comprises cyclosporin A, Oleocal ME-92®, and an oil having a Specific Gravity below 0.9.

Another embodiment comprises cyclosporin A, Oleocal ME-112®, and an oil having a Specific Gravity below 0.9.

Another embodiment comprises cyclosporin A, Oleocal ME-130®, and an oil having a Specific Gravity below 0.9.

Another embodiment comprises cyclosporin A, a Monoglyceride or a Derivative thereof, and an oil having a Specific Gravity below 0.9.

Another embodiment comprises cyclosporin A, Dynacet 211®, and an oil having a Specific Gravity below 0.9.

Another embodiment comprises cyclosporin A, Hetsorb S-20®, and an oil having a Specific Gravity below 0.9.

Another embodiment comprises cyclosporin A, Imwitor 191®, and an oil having a Specific Gravity below 0.9.

Another embodiment comprises cyclosporin A, Imwitor 370®, and an oil having a Specific Gravity below 0.9.

Another embodiment comprises cyclosporin A, Imwitor 375®, and an oil having a Specific Gravity below 0.9.

Another embodiment comprises cyclosporin A, Imwitor 900®, and an oil having a Specific Gravity below 0.9.

Another embodiment comprises cyclosporin A, Imwitor 945®, and an oil having a Specific Gravity below 0.9.

Another embodiment comprises cyclosporin A, Imwitor 2020®, and an oil having a Specific Gravity below 0.9.

Another embodiment comprises cyclosporin A, Kemester 5500®, and an oil having a Specific Gravity below 0.9.

Another embodiment comprises cyclosporin A, Kemester 6000®, and an oil having a Specific Gravity below 0.9.

Another embodiment comprises cyclosporin A, Magrabar GMC®, and an oil having a Specific Gravity below 0.9.

Another embodiment comprises cyclosporin A, Magrabar GMO-CK®, and an oil having a Specific Gravity below 0.9.

Another embodiment comprises cyclosporin A, Magrabar GPC-10®, and an oil having a Specific Gravity below 0.9.

Another embodiment comprises cyclosporin A, Magrabar MDG-5050®, and an oil having a Specific Gravity below 0.9.

Another embodiment comprises cyclosporin A, Monalube 335®, and an oil having a Specific Gravity below 0.9.

Another embodiment comprises cyclosporin A, Monoglycerides Glyceryl Monestearate Archer Daniels Midland Company®, and an oil having a Specific Gravity below 0.9.

Another embodiment comprises cyclosporin A, Rita GMS®, and an oil having a Specific Gravity below 0.9.

Another embodiment comprises cyclosporin A, Ritamulse SCG®, and an oil having a Specific Gravity below 0.9.

Another embodiment comprises cyclosporin A, Softigen 701®, and an oil having a Specific Gravity below 0.9.

Another embodiment comprises cyclosporin A, Tally 100 Plus®, and an oil having a Specific Gravity below 0.9.

Another embodiment comprises cyclosporin A, a Polyethylene Glycol, and an oil having a Specific Gravity below 0.9.

Another embodiment comprises cyclosporin A, Emulgade PL 68/50®, and an oil having a Specific Gravity below 0.9.

Another embodiment comprises cyclosporin A, Lumulse PEG®, and an oil having a Specific Gravity below 0.9.

Another embodiment comprises cyclosporin A, Rhodasurf PEG-400®, and an oil having a Specific Gravity below 0.9.

Another embodiment comprises cyclosporin A, Rhodasurf PEG-600®, and an oil having a Specific Gravity below 0.9.

Another embodiment comprises cyclosporin A, Witconol PEG-400®, and an oil having a Specific Gravity below 0.9.

Another embodiment comprises cyclosporin A, a Polymeric Surfactant, and an oil having a Specific Gravity below 0.9.

Another embodiment comprises cyclosporin A, Acritamer PNC-EG®, and an oil having a Specific Gravity below 0.9.

Another embodiment comprises cyclosporin A, Ag-Rho DEP-775®, and an oil having a Specific Gravity below 0.9.

Another embodiment comprises cyclosporin A, APG 325N Glycoside®, and an oil having a Specific Gravity below 0.9.

Another embodiment comprises cyclosporin A, Aristoflex AVC®, and an oil having a Specific Gravity below 0.9.

Another embodiment comprises cyclosporin A, Aristoflex HMB®, and an oil having a Specific Gravity below 0.9.

Another embodiment comprises cyclosporin A, Burco NPS-225®, and an oil having a Specific Gravity below 0.9.

Another embodiment comprises cyclosporin A, Burco NPS-816®, and an oil having a Specific Gravity below 0.9.

Another embodiment comprises cyclosporin A, Chemccinate 5603®, and an oil having a Specific Gravity below 0.9.

Another embodiment comprises cyclosporin A, Cosmedia Guar C-261N®, and an oil having a Specific Gravity below 0.9.

Another embodiment comprises cyclosporin A, Gantrez S-95®, and an oil having a Specific Gravity below 0.9.

Another embodiment comprises cyclosporin A, Glucopon 220 UP®, and an oil having a Specific Gravity below 0.9.

Another embodiment comprises cyclosporin A, Glucopon 225 DK®, and an oil having a Specific Gravity below 0.9.

Another embodiment comprises cyclosporin A, Glucopon 425 N®, and an oil having a Specific Gravity below 0.9.

Another embodiment comprises cyclosporin A, Glucopon 600 UP®, and an oil having a Specific Gravity below 0.9.

Another embodiment comprises cyclosporin A, Glucopon 625 UP®, and an oil having a Specific Gravity below 0.9.

Another embodiment comprises cyclosporin A, Pemulen 1621®, and an oil having a Specific Gravity below 0.9.

Another embodiment comprises cyclosporin A, Pemulen 1622®, and an oil having a Specific Gravity below 0.9.

Another embodiment comprises cyclosporin A, Pemulen TR-1®, and an oil having a Specific Gravity below 0.9.

Another embodiment comprises cyclosporin A, Pemulen TR-2®, and an oil having a Specific Gravity below 0.9.

Another embodiment comprises cyclosporin A, Plantacare 818®, and an oil having a Specific Gravity below 0.9.

Another embodiment comprises cyclosporin A, Plantapon LGC Sorb®, and an oil having a Specific Gravity below 0.9.

Another embodiment comprises cyclosporin A, Plantaren 1200N®, and an oil having a Specific Gravity below 0.9.

Another embodiment comprises cyclosporin A, Plantaren 2000N®, and an oil having a Specific Gravity below 0.9.

Another embodiment comprises cyclosporin A, Viscolam AT 64®, and an oil having a Specific Gravity below 0.9.

Another embodiment comprises cyclosporin A, Viscolam AT 64P®, and an oil having a Specific Gravity below 0.9.

Another embodiment comprises cyclosporin A, Viscolam AT 100®, and an oil having a Specific Gravity below 0.9.

Another embodiment comprises cyclosporin A, Viscolam MAC 7®, and an oil having a Specific Gravity below 0.9.

Another embodiment comprises cyclosporin A, Viscolam SMC 20®, and an oil having a Specific Gravity below 0.9.

Another embodiment comprises cyclosporin A, Witbreak RTC-323®, and an oil having a Specific Gravity below 0.9.

Another embodiment comprises cyclosporin A, WSI 3700®, and an oil having a Specific Gravity below 0.9.

Another embodiment comprises cyclosporin A, a Propoxylated or Ethoxylated Fatty Acid, Alcohol, or Alkyl Phenol, and an oil having a Specific Gravity below 0.9.

Another embodiment comprises cyclosporin A, Antarox AA-60®, and an oil having a Specific Gravity below 0.9.

Another embodiment comprises cyclosporin A, Antarox LF-224®, and an oil having a Specific Gravity below 0.9.

Another embodiment comprises cyclosporin A, Burcomul DFE-45®, and an oil having a Specific Gravity below 0.9.

Another embodiment comprises cyclosporin A, Burcoterge LFE-1000®, and an oil having a Specific Gravity below 0.9.

Another embodiment comprises cyclosporin A, Chemal LF-25B®, and an oil having a Specific Gravity below 0.9.

Another embodiment comprises cyclosporin A, Chemal LF-40B®, and an oil having a Specific Gravity below 0.9.

Another embodiment comprises cyclosporin A, Dehypon LS-36®, and an oil having a Specific Gravity below 0.9.

Another embodiment comprises cyclosporin A, Dehypon LS-36®, and an oil having a Specific Gravity below 0.9.

Another embodiment comprises cyclosporin A, Dehypon LS-54®, and an oil having a Specific Gravity below 0.9.

Another embodiment comprises cyclosporin A, Dehypon LS-54®, and an oil having a Specific Gravity below 0.9.

Another embodiment comprises cyclosporin A, Delonic 100 VLF®, and an oil having a Specific Gravity below 0.9.

Another embodiment comprises cyclosporin A, Delonic LF-60 MOD®, and an oil having a Specific Gravity below 0.9.

Another embodiment comprises cyclosporin A, Empiderm B®, and an oil having a Specific Gravity below 0.9.

Another embodiment comprises cyclosporin A, Ethylan 1206®, and an oil having a Specific Gravity below 0.9.

Another embodiment comprises cyclosporin A, Ethylan NS-500K®, and an oil having a Specific Gravity below 0.9.

Another embodiment comprises cyclosporin A, Ethylan NS-500LQ®, and an oil having a Specific Gravity below 0.9.

Another embodiment comprises cyclosporin A, Genapol 1392®, and an oil having a Specific Gravity below 0.9.

Another embodiment comprises cyclosporin A, Genapol 2317®, and an oil having a Specific Gravity below 0.9.

Another embodiment comprises cyclosporin A, Genapol 26EP710®, and an oil having a Specific Gravity below 0.9.

Another embodiment comprises cyclosporin A, Genapol EP 1022®, and an oil having a Specific Gravity below 0.9.

Another embodiment comprises cyclosporin A, Genapol EP 1024®, and an oil having a Specific Gravity below 0.9.

Another embodiment comprises cyclosporin A, Genapol EP 6068®, and an oil having a Specific Gravity below 0.9.

Another embodiment comprises cyclosporin A, Genapol NP915®, and an oil having a Specific Gravity below 0.9.

Another embodiment comprises cyclosporin A, Kieralon MFB®, and an oil having a Specific Gravity below 0.9.

Another embodiment comprises cyclosporin A, Lumisolve CSA-80 V®, and an oil having a Specific Gravity below 0.9.

Another embodiment comprises cyclosporin A, Marlowet 5001®, and an oil having a Specific Gravity below 0.9.

Another embodiment comprises cyclosporin A, Marlox FK 64®, and an oil having a Specific Gravity below 0.9.

Another embodiment comprises cyclosporin A, Marlox MO 124®, and an oil having a Specific Gravity below 0.9.

Another embodiment comprises cyclosporin A, Marlox S 58®, and an oil having a Specific Gravity below 0.9.

Another embodiment comprises cyclosporin A, Nonatell 1003®, and an oil having a Specific Gravity below 0.9.

Another embodiment comprises cyclosporin A, Nonatell 1038®, and an oil having a Specific Gravity below 0.9.

Another embodiment comprises cyclosporin A, Nonatell 1052®, and an oil having a Specific Gravity below 0.9.

Another embodiment comprises cyclosporin A, Nonatell 1061®, and an oil having a Specific Gravity below 0.9.

Another embodiment comprises cyclosporin A, Nonatell 1075®, and an oil having a Specific Gravity below 0.9.

Another embodiment comprises cyclosporin A, Nonatell 1088®, and an oil having a Specific Gravity below 0.9.

Another embodiment comprises cyclosporin A, Nonatell 1123®, and an oil having a Specific Gravity below 0.9.

Another embodiment comprises cyclosporin A, Nonatell 1153®, and an oil having a Specific Gravity below 0.9.

Another embodiment comprises cyclosporin A, Nonatell 1161®, and an oil having a Specific Gravity below 0.9.

Another embodiment comprises cyclosporin A, Nonatell 1172®, and an oil having a Specific Gravity below 0.9.

Another embodiment comprises cyclosporin A, Nonatell 1181®, and an oil having a Specific Gravity below 0.9.

Another embodiment comprises cyclosporin A, Norfox 36®, and an oil having a Specific Gravity below 0.9.

Another embodiment comprises cyclosporin A, Procetyl AWS®, and an oil having a Specific Gravity below 0.9.

Another embodiment comprises cyclosporin A, Sandoxylate SX 412®, and an oil having a Specific Gravity below 0.9.

Another embodiment comprises cyclosporin A, Sandoxylate SX 418®, and an oil having a Specific Gravity below 0.9.

Another embodiment comprises cyclosporin A, Surfonic JL-80X®, and an oil having a Specific Gravity below 0.9.

Another embodiment comprises cyclosporin A, Surfonic JL-80X-B1®, and an oil having a Specific Gravity below 0.9.

Another embodiment comprises cyclosporin A, Sunfonic L4-29X®, and an oil having a Specific Gravity below 0.9.

Another embodiment comprises cyclosporin A, Surfonic LF®, and an oil having a Specific Gravity below 0.9.

Another embodiment comprises cyclosporin A, T-Det A826®, and an oil having a Specific Gravity below 0.9.

Another embodiment comprises cyclosporin A, T-Det LF-416®, and an oil having a Specific Gravity below 0.9.

Another embodiment comprises cyclosporin A, Tergitol Min-Foam 1X®, and an oil having a Specific Gravity below 0.9.

Another embodiment comprises cyclosporin A, Tergitol Min-Foam 2X®, and an oil having a Specific Gravity below 0.9.

Another embodiment comprises cyclosporin A, Triton CF-21®, and an oil having a Specific Gravity below 0.9.

Another embodiment comprises cyclosporin A, Triton CF-76®, and an oil having a Specific Gravity below 0.9.

Another embodiment comprises cyclosporin A, Triton XL-80N®, and an oil having a Specific Gravity below 0.9.

Another embodiment comprises cyclosporin A, Witconol NS-98®, and an oil having a Specific Gravity below 0.9.

Another embodiment comprises cyclosporin A, Witconol NS-108Q®, and an oil having a Specific Gravity below 0.9.

Another embodiment comprises cyclosporin A, Witconol NS-145®, and an oil having a Specific Gravity below 0.9.

Another embodiment comprises cyclosporin A, Witconol NS-179®, and an oil having a Specific Gravity below 0.9.

Another embodiment comprises cyclosporin A, a Protein-based Surfactant, and an oil having a Specific Gravity below 0.9.

Another embodiment comprises cyclosporin A, AminoFoam W®, and an oil having a Specific Gravity below 0.9.

Another embodiment comprises cyclosporin A, Amiter LGOD-2®, and an oil having a Specific Gravity below 0.9.

Another embodiment comprises cyclosporin A, Amiter LGS-2®, and an oil having a Specific Gravity below 0.9.

Another embodiment comprises cyclosporin A, Amiter LGS-5®, and an oil having a Specific Gravity below 0.9.

Another embodiment comprises cyclosporin A, Lamepon S®, and an oil having a Specific Gravity below 0.9.

Another embodiment comprises cyclosporin A, Lamepon S®, and an oil having a Specific Gravity below 0.9.

Another embodiment comprises cyclosporin A, Maypon 4C®, and an oil having a Specific Gravity below 0.9.

Another embodiment comprises cyclosporin A, May-Tein C®, and an oil having a Specific Gravity below 0.9.

Another embodiment comprises cyclosporin A, May-Tein CT®, and an oil having a Specific Gravity below 0.9.

Another embodiment comprises cyclosporin A, May-Tein KTS®, and an oil having a Specific Gravity below 0.9.

Another embodiment comprises cyclosporin A, May-Tein SY®, and an oil having a Specific Gravity below 0.9.

Another embodiment comprises cyclosporin A, Plantapon S®, and an oil having a Specific Gravity below 0.9.

Another embodiment comprises cyclosporin A, Proteol APL®, and an oil having a Specific Gravity below 0.9.

Another embodiment comprises cyclosporin A, Proteol OAT®, and an oil having a Specific Gravity below 0.9.

Another embodiment comprises cyclosporin A, Pyroter CPI-40®, and an oil having a Specific Gravity below 0.9.

Another embodiment comprises cyclosporin A, Pyroter GPI-25®, and an oil having a Specific Gravity below 0.9.

Another embodiment comprises cyclosporin A, Supro-Tein S®, and an oil having a Specific Gravity below 0.9.

Another embodiment comprises cyclosporin A, Supro-Tein V®, and an oil having a Specific Gravity below 0.9.

Another embodiment comprises cyclosporin A, a Sarcosine Derivative®, and an oil having a Specific Gravity below 0.9.

Another embodiment comprises cyclosporin A, Crodasinic LS-30®, and an oil having a Specific Gravity below 0.9.

Another embodiment comprises cyclosporin A, Vanseal CS®, and an oil having a Specific Gravity below 0.9.

Another embodiment comprises cyclosporin A, Vanseal LS®, and an oil having a Specific Gravity below 0.9.

Another embodiment comprises cyclosporin A, Vanseal MS®, and an oil having a Specific Gravity below 0.9.

Another embodiment comprises cyclosporin A, Vanseal NACS-30®, and an oil having a Specific Gravity below 0.9.

Another embodiment comprises cyclosporin A, Vanseal NALS-95®, and an oil having a Specific Gravity below 0.9.

Another embodiment comprises cyclosporin A, Vanseal OS®, and an oil having a Specific Gravity below 0.9.

Another embodiment comprises cyclosporin A, a Silicone-based Surfactant®, and an oil having a Specific Gravity below 0.9.

Another embodiment comprises cyclosporin A, Abil-B-9950®, and an oil having a Specific Gravity below 0.9.

Another embodiment comprises cyclosporin A, Abil Care 85®, and an oil having a Specific Gravity below 0.9.

Another embodiment comprises cyclosporin A, Abil EM 90®, and an oil having a Specific Gravity below 0.9.

Another embodiment comprises cyclosporin A, Abil EM 97®, and an oil having a Specific Gravity below 0.9.

Another embodiment comprises cyclosporin A, Abil WE-09®, and an oil having a Specific Gravity below 0.9.

Another embodiment comprises cyclosporin A, Dow Corning 1248 Fluid®, and an oil having a Specific Gravity below 0.9.

Another embodiment comprises cyclosporin A, Dow Corning 3225C®, and an oil having a Specific Gravity below 0.9.

Another embodiment comprises cyclosporin A, Dow Corning 5200®, and an oil having a Specific Gravity below 0.9.

Another embodiment comprises cyclosporin A, Dow Corning Q4-3667®, and an oil having a Specific Gravity below 0.9.

Another embodiment comprises cyclosporin A, Monasil PCA®, and an oil having a Specific Gravity below 0.9.

Another embodiment comprises cyclosporin A, Monasil PDM®, and an oil having a Specific Gravity below 0.9.

Another embodiment comprises cyclosporin A, Monasil PLN®, and an oil having a Specific Gravity below 0.9.

Another embodiment comprises cyclosporin A, Polyderm PPI-SI-WS®, and an oil having a Specific Gravity below 0.9.

Another embodiment comprises cyclosporin A, Troysol 380W®, and an oil having a Specific Gravity below 0.9.

Another embodiment comprises cyclosporin A, Troysol S366®, and an oil having a Specific Gravity below 0.9.

Another embodiment comprises cyclosporin A, a Sorbitan Derivative, and an oil having a Specific Gravity below 0.9.

Another embodiment comprises cyclosporin A, Alkamuls SML®, and an oil having a Specific Gravity below 0.9.

Another embodiment comprises cyclosporin A, Alkamuls SMO®, and an oil having a Specific Gravity below 0.9.

Another embodiment comprises cyclosporin A, Alkamuls STO®, and an oil having a Specific Gravity below 0.9.

Another embodiment comprises cyclosporin A, Arlacel 20®, and an oil having a Specific Gravity below 0.9.

Another embodiment comprises cyclosporin A, Arlacel 40®, and an oil having a Specific Gravity below 0.9.

Another embodiment comprises cyclosporin A, Arlacel 60®, and an oil having a Specific Gravity below 0.9.

Another embodiment comprises cyclosporin A, Arlacel 80®, and an oil having a Specific Gravity below 0.9.

Another embodiment comprises cyclosporin A, Arlacel C®, and an oil having a Specific Gravity below 0.9.

Another embodiment comprises cyclosporin A, Armul 21®, and an oil having a Specific Gravity below 0.9.

Another embodiment comprises cyclosporin A, Atlox 80®, and an oil having a Specific Gravity below 0.9.

Another embodiment comprises cyclosporin A, Atlox 847®, and an oil having a Specific Gravity below 0.9.

Another embodiment comprises cyclosporin A, Atlox 1045A®, and an oil having a Specific Gravity below 0.9.

Another embodiment comprises cyclosporin A, Canarcel 20®, and an oil having a Specific Gravity below 0.9.

Another embodiment comprises cyclosporin A, Canarcel 60®, and an oil having a Specific Gravity below 0.9.

Another embodiment comprises cyclosporin A, Canarcel 80®, and an oil having a Specific Gravity below 0.9.

Another embodiment comprises cyclosporin A, Canarcel TW 20®, and an oil having a Specific Gravity below 0.9.

Another embodiment comprises cyclosporin A, Canarcel TW 60®, and an oil having a Specific Gravity below 0.9.

Another embodiment comprises cyclosporin A, Canarcel TW 80®, and an oil having a Specific Gravity below 0.9.

Another embodiment comprises cyclosporin A, Coladet BSB-P®, and an oil having a Specific Gravity below 0.9.

Another embodiment comprises cyclosporin A, Customulse O-20®, and an oil having a Specific Gravity below 0.9.

Another embodiment comprises cyclosporin A, Dehymuls E®, and an oil having a Specific Gravity below 0.9.

Another embodiment comprises cyclosporin A, DeSotan SMO®, and an oil having a Specific Gravity below 0.9.

Another embodiment comprises cyclosporin A, DeSotan SMO-20®, and an oil having a Specific Gravity below 0.9.

Another embodiment comprises cyclosporin A, DeSotan SMT®, and an oil having a Specific Gravity below 0.9.

Another embodiment comprises cyclosporin A, DeSotan SMT-20®, and an oil having a Specific Gravity below 0.9.

Another embodiment comprises cyclosporin A, Durfax 60®, and an oil having a Specific Gravity below 0.9.

Another embodiment comprises cyclosporin A, Durfax 65®, and an oil having a Specific Gravity below 0.9.

Another embodiment comprises cyclosporin A, Durfax 80®, and an oil having a Specific Gravity below 0.9.

Another embodiment comprises cyclosporin A, Durtan 60®, and an oil having a Specific Gravity below 0.9.

Another embodiment comprises cyclosporin A, Durtan 65®, and an oil having a Specific Gravity below 0.9.

Another embodiment comprises cyclosporin A, Liposorb L®, and an oil having a Specific Gravity below 0.9.

Another embodiment comprises cyclosporin A, Liposorb L-10®, and an oil having a Specific Gravity below 0.9.

Another embodiment comprises cyclosporin A, Liposorb L-20®, and an oil having a Specific Gravity below 0.9.

Another embodiment comprises cyclosporin A, Liposorb O®, and an oil having a Specific Gravity below 0.9.

Another embodiment comprises cyclosporin A, Liposorb O-20®, and an oil having a Specific Gravity below 0.9.

Another embodiment comprises cyclosporin A, Liposorb P®, and an oil having a Specific Gravity below 0.9.

Another embodiment comprises cyclosporin A, Liposorb P-20®, and an oil having a Specific Gravity below 0.9.

Another embodiment comprises cyclosporin A, Liposorb S®, and an oil having a Specific Gravity below 0.9.

Another embodiment comprises cyclosporin A, Liposorb S-20®, and an oil having a Specific Gravity below 0.9.

Another embodiment comprises cyclosporin A, Liposorb SQO®, and an oil having a Specific Gravity below 0.9.

Another embodiment comprises cyclosporin A, Liposorb TO®, and an oil having a Specific Gravity below 0.9.

Another embodiment comprises cyclosporin A, Liposorb TS®, and an oil having a Specific Gravity below 0.9.

Another embodiment comprises cyclosporin A, Liposorb TS-20®, and an oil having a Specific Gravity below 0.9.

Another embodiment comprises cyclosporin A, Lumisorb PS®, and an oil having a Specific Gravity below 0.9.

Another embodiment comprises cyclosporin A, Lumisorb SMO (T)®, and an oil having a Specific Gravity below 0.9.

Another embodiment comprises cyclosporin A, Lumisorb SMS K®, and an oil having a Specific Gravity below 0.9.

Another embodiment comprises cyclosporin A, Lumisorb SSO®, and an oil having a Specific Gravity below 0.9.

Another embodiment comprises cyclosporin A, Lumisorb STS K®, and an oil having a Specific Gravity below 0.9.

Another embodiment comprises cyclosporin A, Lumisorb STT®, and an oil having a Specific Gravity below 0.9.

Another embodiment comprises cyclosporin A, Magrabar SMO®, and an oil having a Specific Gravity below 0.9.

Another embodiment comprises cyclosporin A, Magrabar SMO-VEG®, and an oil having a Specific Gravity below 0.9.

Another embodiment comprises cyclosporin A, Magrabar SMT®, and an oil having a Specific Gravity below 0.9.

Another embodiment comprises cyclosporin A, Magrabar STO®, and an oil having a Specific Gravity below 0.9.

Another embodiment comprises cyclosporin A, Miracare BC-27®, and an oil having a Specific Gravity below 0.9.

Another embodiment comprises cyclosporin A, Ritabate 20®, and an oil having a Specific Gravity below 0.9.

Another embodiment comprises cyclosporin A, Ritabate 40®, and an oil having a Specific Gravity below 0.9.

Another embodiment comprises cyclosporin A, Ritabate 60®, and an oil having a Specific Gravity below 0.9.

Another embodiment comprises cyclosporin A, Ritabate 80®, and an oil having a Specific Gravity below 0.9.

Another embodiment comprises cyclosporin A, T-Maz®, and an oil having a Specific Gravity below 0.9.

Another embodiment comprises cyclosporin A, Tego SML®, and an oil having a Specific Gravity below 0.9.

Another embodiment comprises cyclosporin A, Tego SML 20®, and an oil having a Specific Gravity below 0.9.

Another embodiment comprises cyclosporin A, Tego SMO 80 V®, and an oil having a Specific Gravity below 0.9.

Another embodiment comprises cyclosporin A, Tego SMO V®, and an oil having a Specific Gravity below 0.9.

Another embodiment comprises cyclosporin A, Tego SMS®, and an oil having a Specific Gravity below 0.9.

Another embodiment comprises cyclosporin A, Tego STO V®, and an oil having a Specific Gravity below 0.9.

Another embodiment comprises cyclosporin A, Tween 21®, and an oil having a Specific Gravity below 0.9.

Another embodiment comprises cyclosporin A, Tween 40®, and an oil having a Specific Gravity below 0.9.

Another embodiment comprises cyclosporin A, Tween 60®, and an oil having a Specific Gravity below 0.9.

Another embodiment comprises cyclosporin A, Tween 60 K®, and an oil having a Specific Gravity below 0.9.

Another embodiment comprises cyclosporin A, Tween 61®, and an oil having a Specific Gravity below 0.9.

Another embodiment comprises cyclosporin A, Tween 65®, and an oil having a Specific Gravity below 0.9.

Another embodiment comprises cyclosporin A, Tween 80®, and an oil having a Specific Gravity below 0.9.

Another embodiment comprises cyclosporin A, Tween 80 K®, and an oil having a Specific Gravity below 0.9.

Another embodiment comprises cyclosporin A, Tween 81®, and an oil having a Specific Gravity below 0.9.

Another embodiment comprises cyclosporin A, Tween 85®, and an oil having a Specific Gravity below 0.9.

Another embodiment comprises cyclosporin A, a Sucrose or Glucose Ester, or Derivative thereof, and an oil having a Specific Gravity below 0.9.

Another embodiment comprises cyclosporin A, DeSulf GOS-P-60WCG®, and an oil having a Specific Gravity below 0.9.

Another embodiment comprises cyclosporin A, Glucam E-20 Distearate®, and an oil having a Specific Gravity below 0.9.

Another embodiment comprises cyclosporin A, Glucamate DOE-120®, and an oil having a Specific Gravity below 0.9.

Another embodiment comprises cyclosporin A, Glucamate SSE-20®, and an oil having a Specific Gravity below 0.9.

Another embodiment comprises cyclosporin A, Glucate DO®, and an oil having a Specific Gravity below 0.9.

Another embodiment comprises cyclosporin A, Glucate SS®, and an oil having a Specific Gravity below 0.9.

Another embodiment comprises cyclosporin A, Glucopon 425 UP®, and an oil having a Specific Gravity below 0.9.

Another embodiment comprises cyclosporin A, Isolan IS®, and an oil having a Specific Gravity below 0.9.

Another embodiment comprises cyclosporin A, Mazon 40®, and an oil having a Specific Gravity below 0.9.

Another embodiment comprises cyclosporin A, Montanov 82®, and an oil having a Specific Gravity below 0.9.

Another embodiment comprises cyclosporin A, Montanov 202®, and an oil having a Specific Gravity below 0.9.

Another embodiment comprises cyclosporin A, Montanov S®, and an oil having a Specific Gravity below 0.9.

Another embodiment comprises cyclosporin A, Rheozan®, and an oil having a Specific Gravity below 0.9.

Another embodiment comprises cyclosporin A, Simulsol AS 48®, and an oil having a Specific Gravity below 0.9.

Another embodiment comprises cyclosporin A, Simulsol SL 4®, and an oil having a Specific Gravity below 0.9.

Another embodiment comprises cyclosporin A, Simulsol SL 10®, and an oil having a Specific Gravity below 0.9.

Another embodiment comprises cyclosporin A, Simulsol SL 11W®, and an oil having a Specific Gravity below 0.9.

Another embodiment comprises cyclosporin A, Simulsol SL 55®, and an oil having a Specific Gravity below 0.9.

Another embodiment comprises cyclosporin A, Suga Nate 100 and 160®, and an oil having a Specific Gravity below 0.9.

Another embodiment comprises cyclosporin A, Tego Care 450®, and an oil having a Specific Gravity below 0.9.

Another embodiment comprises cyclosporin A, Tego Care CG 90®, and an oil having a Specific Gravity below 0.9.

Another embodiment comprises cyclosporin A, Tego Care PS®, and an oil having a Specific Gravity below 0.9.

Another embodiment comprises cyclosporin A, Tegosoft PSE 141 G®, and an oil having a Specific Gravity below 0.9.

Another embodiment comprises cyclosporin A, Tegotens G 826®, and an oil having a Specific Gravity below 0.9.

Another embodiment comprises cyclosporin A, Triton BG-10 (70%)®, and an oil having a Specific Gravity below 0.9.

Another embodiment comprises cyclosporin A, Triton CG-110 (60%)®, and an oil having a Specific Gravity below 0.9.

Another embodiment comprises cyclosporin A, Wickenol 545®, and an oil having a Specific Gravity below 0.9.

Another embodiment comprises cyclosporin A, an alcohol, and Mineral oil.

Another embodiment comprises cyclosporin A, Diglycerol®, and Mineral oil.

Another embodiment comprises cyclosporin A, Hetoxide GT-80®, and Mineral oil.

Another embodiment comprises cyclosporin A, Lexemul BEO®, and Mineral oil.

Another embodiment comprises cyclosporin A, Polyglycerol-3®, and Mineral oil.

Another embodiment comprises cyclosporin A, Redicote E Series®, and Mineral oil.

Another embodiment comprises cyclosporin A, Simulsol OX 1005L®, and Mineral oil.

Another embodiment comprises cyclosporin A, Stanfax 567®, and Mineral oil.

Another embodiment comprises cyclosporin A, TA-1618®, and Mineral oil.

Another embodiment comprises cyclosporin A, Witconol H-31A®, and Mineral oil.

Another embodiment comprises cyclosporin A, an amine oxide, and Mineral oil.

Another embodiment comprises cyclosporin A, AO-405®, and Mineral oil.

Another embodiment comprises cyclosporin A, AO-455®, and Mineral oil.

Another embodiment comprises cyclosporin A, AO 728 Special®, and Mineral oil.

Another embodiment comprises cyclosporin A, Barlox 12®, and Mineral oil.

Another embodiment comprises cyclosporin A, Barlox 14®, and Mineral oil.

Another embodiment comprises cyclosporin A, Burcoxide Lo®, and Mineral oil.

Another embodiment comprises cyclosporin A, Caloxamine LO®, and Mineral oil.

Another embodiment comprises cyclosporin A, Chemoxide CAW®, and Mineral oil.

Another embodiment comprises cyclosporin A, Chemoxide LM-30®, and Mineral oil.

Another embodiment comprises cyclosporin A, Chemoxide LO®, and Mineral oil.

Another embodiment comprises cyclosporin A, Chemoxide MO®, and Mineral oil.

Another embodiment comprises cyclosporin A, Colalux CAO-35®, and Mineral oil.

Another embodiment comprises cyclosporin A, Colalux LO®, and Mineral oil.

Another embodiment comprises cyclosporin A, DeMox CAPO®, and Mineral oil.

Another embodiment comprises cyclosporin A, DeMox CSG-30®, and Mineral oil.

Another embodiment comprises cyclosporin A, DeMox LAO®, and Mineral oil.

Another embodiment comprises cyclosporin A, Emcol L®, and Mineral oil.

Another embodiment comprises cyclosporih A, Empigen OB®, and Mineral oil.

Another embodiment comprises cyclosporin A, Empigen OS/A®, and Mineral oil.

Another embodiment comprises cyclosporin A, Foamox CDO®, and Mineral oil.

Another embodiment comprises cyclosporin A, Foamox DMM®, and Mineral oil.

Another embodiment comprises cyclosporin A, Foamox DMS®, and Mineral oil.

Another embodiment comprises cyclosporin A, Genaminox KC®, and Mineral oil.

Another embodiment comprises cyclosporin A, Genaminox LA®, and Mineral oil.

Another embodiment comprises cyclosporin A, Hartofoam SAO®, and Mineral oil.

Another embodiment comprises cyclosporin A, Hartox DMCD®, and Mineral oil.

Another embodiment comprises cyclosporin A, Lipowax DAT®, and Mineral oil.

Another embodiment comprises cyclosporin A, Lipowax PB Pastilles®, and Mineral oil.

Another embodiment comprises cyclosporin A, Mackamine C8®, and Mineral oil.

Another embodiment comprises cyclosporin A, Mackamine C10®, and Mineral oil.

Another embodiment comprises cyclosporin A, Mackamine C14®, and Mineral oil.

Another embodiment comprises cyclosporin A, Mackamine CAO®, and Mineral oil.

Another embodiment comprises cyclosporin A, Mackamine CO®, and Mineral oil.

Another embodiment comprises cyclosporin A, Mackamine LO®, and Mineral oil.

Another embodiment comprises cyclosporin A, Mackamine O2®, and Mineral oil.

Another embodiment comprises cyclosporin A, Mackamine SAO®, and Mineral oil.

Another embodiment comprises cyclosporin A, Mackamine SO®, and Mineral oil.

Another embodiment comprises cyclosporin A, Mazox KCAO®, and Mineral oil.

Another embodiment comprises cyclosporin A, Monalac MO®, and Mineral oil.

Another embodiment comprises cyclosporin A, Norfox LDA®, and Mineral oil.

Another embodiment comprises cyclosporin A, Rhodamox LO®, and Mineral oil.

Another embodiment comprises cyclosporin A, Schercamox C-AA®, and Mineral oil.

Another embodiment comprises cyclosporin A, Schercamox DMA®, and Mineral oil.

Another embodiment comprises cyclosporin A, Schercamox DML®, and Mineral oil.

Another embodiment comprises cyclosporin A, Schercamox DMM®, and Mineral oil.

Another embodiment comprises cyclosporin A, Schercamox DMS®, and Mineral oil.

Another embodiment comprises cyclosporin A, Tegotens DO®, and Mineral oil.

Another embodiment comprises cyclosporin A, Tomah AO-14-2®, and Mineral oil.

Another embodiment comprises cyclosporin A, Triaminox CDO®, and Mineral oil.

Another embodiment comprises cyclosporin A, a block polymer, and Mineral oil.

Another embodiment comprises cyclosporin A, AL 2070®, and Mineral oil.

Another embodiment comprises cyclosporin A, Antarox 17-R-2®, and Mineral oil.

Another embodiment comprises cyclosporin A, Antarox 25-R-2®, and Mineral oil.

Another embodiment comprises cyclosporin A, Antarox 31-R-1®, and Mineral oil.

Another embodiment comprises cyclosporin A, Antarox P-84®, and Mineral oil.

Another embodiment comprises cyclosporin A, Antarox P-104/H®, and Mineral oil.

Another embodiment comprises cyclosporin A, Arnox BP-Series®, and Mineral oil.

Another embodiment comprises cyclosporin A, Chemonic 435®, and Mineral oil.

Another embodiment comprises cyclosporin A, Chemonic D-25®, and Mineral oil.

Another embodiment comprises cyclosporin A, Chemonic PL Series®, and Mineral oil.

Another embodiment comprises cyclosporin A, Ethox L-121®, and Mineral oil.

Another embodiment comprises cyclosporin A, Ethox L-122®, and Mineral oil.

Another embodiment comprises cyclosporin A, Genapol PF-10®, and Mineral oil.

Another embodiment comprises cyclosporin A, Genapol PF-20®, and Mineral oil.

Another embodiment comprises cyclosporin A, Genapol PF-40A®, and Mineral oil.

Another embodiment comprises cyclosporin A, Norfox 2-LF®, and Mineral oil.

Another embodiment comprises cyclosporin A, Simulsol NW 342®, and Mineral oil.

Another embodiment comprises cyclosporin A, T-Det BP-1®, and Mineral oil.

Another embodiment comprises cyclosporin A, T-Det XD®, and Mineral oil.

Another embodiment comprises cyclosporin A, T-Det XH®, and Mineral oil.

Another embodiment comprises cyclosporin A, Triton CF-32®, and Mineral oil.

Another embodiment comprises cyclosporin A, Witconol 171®, and Mineral oil.

Another embodiment comprises cyclosporin A, Witconol 324®, and Mineral oil.

Another embodiment comprises cyclosporin A, Witconol 324D®, and Mineral oil.

Another embodiment comprises cyclosporin A, Witconol PD-2000®, and Mineral oil.

Another embodiment comprises cyclosporin A, a Carboxylated Alcohol or Alkylphenol Ethoxylate, and Mineral oil.

Another embodiment comprises cyclosporin A, Emcol CN-6®, and Mineral oil.

Another embodiment comprises cyclosporin A, Ethcarb®, and Mineral oil.

Another embodiment comprises cyclosporin A, Gemtex WNT-Conc®, and Mineral oil.

Another embodiment comprises cyclosporin A, Incrodet TD7-C®, and Mineral oil.

Another embodiment comprises cyclosporin A, Marlinat CM 105/80®, and Mineral oil.

Another embodiment comprises cyclosporin A, Marlowet 1072®, and Mineral oil.

Another embodiment comprises cyclosporin A, Marlowet 4530®, and Mineral oil.

Another embodiment comprises cyclosporin A, Marlowet 4530 LF®, and Mineral oil.

Another embodiment comprises cyclosporin A, Marlowet 4534®, and Mineral oil.

Another embodiment comprises cyclosporin A, Marlowet 4538®, and Mineral oil.

Another embodiment comprises cyclosporin A, Marlowet 4539®, and Mineral oil.

Another embodiment comprises cyclosporin A, Marlowet 4539 LF®, and Mineral oil.

Another embodiment comprises cyclosporin A, Marlowet 4541®, and Mineral oil.

Another embodiment comprises cyclosporin A, Miranate LEC-80®, and Mineral oil.

Another embodiment comprises cyclosporin A, Sandopan B®, and Mineral oil.

Another embodiment comprises cyclosporin A, Sandopan B Modified®, and Mineral oil.

Another embodiment comprises cyclosporin A, Sandopan LS-24 Gel®, and Mineral oil.

Another embodiment comprises cyclosporin A, Surfine T-A®, and Mineral oil.

Another embodiment comprises cyclosporin A, a Carboxylic Acid/Fatty Acid, and Mineral oil.

Another embodiment comprises cyclosporin A, Colaterge RAM®, and Mineral oil.

Another embodiment comprises cyclosporin A, Colatrope INC®, and Mineral oil.

Another embodiment comprises cyclosporin A, Crodacid B®, and Mineral oil.

Another embodiment comprises cyclosporin A, DeTrope CA-100®, and Mineral oil.

Another embodiment comprises cyclosporin A, Latol MTO®, and Mineral oil.

Another embodiment comprises cyclosporin A, Lumulse CC-33 K®, and Mineral oil.

Another embodiment comprises cyclosporin A, Mulls 2218®, and Mineral oil.

Another embodiment comprises cyclosporin A, OL-600®, and Mineral oil.

Another embodiment comprises cyclosporin A, OL-800®, and Mineral oil.

Another embodiment comprises cyclosporin A, R-910®, and Mineral oil.

Another embodiment comprises cyclosporin A, S-210®, and Mineral oil.

Another embodiment comprises cyclosporin A, Sandopan DTC Acid®, and Mineral oil.

Another embodiment comprises cyclosporin A, Sandopan LS 24 N®, and Mineral oil.

Another embodiment comprises cyclosporin A, Sandopan MA-18®, and Mineral oil.

Another embodiment comprises cyclosporin A, an Ethoxylated Alcohol, and Mineral oil.

Another embodiment comprises cyclosporin A, Adsee 799®, and Mineral oil.

Another embodiment comprises cyclosporin A, Adsee 799®, and Mineral oil.

Another embodiment comprises cyclosporin A, Alfonic 610-3.5®, and Mineral oil.

Another embodiment comprises cyclosporin A, Alfonic 810-2®, and Mineral oil.

Another embodiment comprises cyclosporin A, Alfonic 810-6®, and Mineral oil.

Another embodiment comprises cyclosporin A, Alfonic 1012-3®, and Mineral oil.

Another embodiment comprises cyclosporin A, Alfonic 1012-5®, and Mineral oil.

Another embodiment comprises cyclosporin A, Alfonic 1216CO-1.5®, and Mineral oil.

Another embodiment comprises cyclosporin A, Alfonic 1216CO-7®, and Mineral oil.

Another embodiment comprises cyclosporin A, Alfonic 1412-3®, and Mineral oil.

Another embodiment comprises cyclosporin A, Alfonic 1412-7®, and Mineral oil.

Another embodiment comprises cyclosporin A, Arlasolve 200®, and Mineral oil.

Another embodiment comprises cyclosporin A, Arlasolve 200 Liquid®, and Mineral oil.

Another embodiment comprises cyclosporin A, Armix 180-C®, and Mineral oil.

Another embodiment comprises cyclosporin A, Armix 183®, and Mineral oil.

Another embodiment comprises cyclosporin A, Armul 2404®, and Mineral oil.

Another embodiment comprises cyclosporin A, Armul 2404®, and Mineral oil.

Another embodiment comprises cyclosporin A, Atlas EMJ-C®, and Mineral oil.

Another embodiment comprises cyclosporin A, Atlas G-2109®, and Mineral oil.

Another embodiment comprises cyclosporin A, Atlas G-3886®, and Mineral oil.

Another embodiment comprises cyclosporin A, Atlas G-3890®, and Mineral oil.

Another embodiment comprises cyclosporin A, Bio Soft E-200®, and Mineral oil.

Another embodiment comprises cyclosporin A, Bio Soft E-300®, and Mineral oil.

Another embodiment comprises cyclosporin A, Bio Soft E-400®, and Mineral oil.

Another embodiment comprises cyclosporin A, Bio Soft EN 600®, and Mineral oil.

Another embodiment comprises cyclosporin A, Bio Soft TD-400®, and Mineral oil.

Another embodiment comprises cyclosporin A, Bio Soft TD-630®, and Mineral oil.

Another embodiment comprises cyclosporin A, Brij 30®, and Mineral oil.

Another embodiment comprises cyclosporin A, Brij 52®, and Mineral oil.

Another embodiment comprises cyclosporin A, Brij 56®, and Mineral oil.

Another embodiment comprises cyclosporin A, Brij 58®, and Mineral oil.

Another embodiment comprises cyclosporin A, Brij 72®, and Mineral oil.

Another embodiment comprises cyclosporin A, Brij 76®, and Mineral oil.

Another embodiment comprises cyclosporin A, Brij 78®, and Mineral oil.

Another embodiment comprises cyclosporin A, Brij 93®, and Mineral oil.

Another embodiment comprises cyclosporin A, Brij 97®, and Mineral oil.

Another embodiment comprises cyclosporin A, Brij 98®, and Mineral oil.

Another embodiment comprises cyclosporin A, Brij 700®, and Mineral oil.

Another embodiment comprises cyclosporin A, Brij 700 S®, and Mineral oil.

Another embodiment comprises cyclosporin A, Brij 721®, and Mineral oil.

Another embodiment comprises cyclosporin A, Brij 721 S®, and Mineral oil.

Another embodiment comprises cyclosporin A, Burcoterge CDG®, and Mineral oil.

Another embodiment comprises cyclosporin A, Canasol AT 600®, and Mineral oil.

Another embodiment comprises cyclosporin A, Canasol AT 800®, and Mineral oil.

Another embodiment comprises cyclosporin A, Canasol AT 1200®, and Mineral oil.

Another embodiment comprises cyclosporin A, Canasol BJ 35®, and Mineral oil.

Another embodiment comprises cyclosporin A, Canasol BJ 36®, and Mineral oil.

Another embodiment comprises cyclosporin A, Canasol BJ 52®, and Mineral oil.

Another embodiment comprises cyclosporin A, Canasol BJ 58®, and Mineral oil.

Another embodiment comprises cyclosporin A, Canasol BJ 72®, and Mineral oil.

Another embodiment comprises cyclosporin A, Canasol BJ 78®, and Mineral oil.

Another embodiment comprises cyclosporin A, Canasol BJ 98®, and Mineral oil.

Another embodiment comprises cyclosporin A, Canasol BJ 307®, and Mineral oil.

Another embodiment comprises cyclosporin A, Cerfak 1400®, and Mineral oil.

Another embodiment comprises cyclosporin A, Cetomacrogol 1000 BP®, and Mineral oil.

Another embodiment comprises cyclosporin A, Chemonic C-2®, and Mineral oil.

Another embodiment comprises cyclosporin A, Chemonic C-10®, and Mineral oil.

Another embodiment comprises cyclosporin A, Chemonic C-20®, and Mineral oil.

Another embodiment comprises cyclosporin A, Chemonic CT-12®, and Mineral oil.

Another embodiment comprises cyclosporin A, Chemonic CT-20®, and Mineral oil.

Another embodiment comprises cyclosporin A, Chemonic CT-30®, and Mineral oil.

Another embodiment comprises cyclosporin A, Chemonic CT-55®, and Mineral oil.

Another embodiment comprises cyclosporin A, Chemonic G-7®, and Mineral oil.

Another embodiment comprises cyclosporin A, Chemonic G-26®, and Mineral oil.

Another embodiment comprises cyclosporin A, Chemonic L-4®, and Mineral oil.

Another embodiment comprises cyclosporin A, Chemonic L-7®, and Mineral oil.

Another embodiment comprises cyclosporin A, Chemonic L-12®, and Mineral oil.

Another embodiment comprises cyclosporin A, Chemonic L-23®, and Mineral oil.

Another embodiment comprises cyclosporin A, Chemonic O-2®, and Mineral oil.

Another embodiment comprises cyclosporin A, Chemonic O-5®, and Mineral oil.

Another embodiment comprises cyclosporin A, Chemonic O-10®, and Mineral oil.

Another embodiment comprises cyclosporin A, Chemonic O-20®, and Mineral oil.

Another embodiment comprises cyclosporin A, Chemonic S-2®, and Mineral oil.

Another embodiment comprises cyclosporin A, Chemonic S-10®, and Mineral oil.

Another embodiment comprises cyclosporin A, Chemonic S-20®, and Mineral oil.

Another embodiment comprises cyclosporin A, Colamulse FE®, and Mineral oil.

Another embodiment comprises cyclosporin A, Cremophor A 20®, and Mineral oil.

Another embodiment comprises cyclosporin A, Cremophor SA 2®, and Mineral oil.

Another embodiment comprises cyclosporin A, Dehydol 100®, and Mineral oil.

Another embodiment comprises cyclosporin A, Dehydol O-4®, and Mineral oil.

Another embodiment comprises cyclosporin A, Delonic C-18®, and Mineral oil.

Another embodiment comprises cyclosporin A, DeSonic 6T®, and Mineral oil.

Another embodiment comprises cyclosporin A, DeSonic 9D®, and Mineral oil.

Another embodiment comprises cyclosporin A, DeSonic 9T®, and Mineral oil.

Another embodiment comprises cyclosporin A, DeSonic 12D®, and Mineral oil.

Another embodiment comprises cyclosporin A, DeSonic 12T®, and Mineral oil.

Another embodiment comprises cyclosporin A, DeSonic 15T®, and Mineral oil.

Another embodiment comprises cyclosporin A, DeSonic TDA-9®, and Mineral oil.

Another embodiment comprises cyclosporin A, DeThox GLG-7®, and Mineral oil.

Another embodiment comprises cyclosporin A, DeThox GLG-26®, and Mineral oil.

Another embodiment comprises cyclosporin A, DeThox LA-4®, and Mineral oil.

Another embodiment comprises cyclosporin A, DeThox LA-23®, and Mineral oil.

Another embodiment comprises cyclosporin A, DeThox SA-80®, and Mineral oil.

Another embodiment comprises cyclosporin A, Disponil O5®, and Mineral oil.

Another embodiment comprises cyclosporin A, Eccoterge EO-41B®, and Mineral oil.

Another embodiment comprises cyclosporin A, Empilan KA2.5/90®, and Mineral oil.

Another embodiment comprises cyclosporin A, Empilan KA5/90®, and Mineral oil.

Another embodiment comprises cyclosporin A, Empilan KM-20®, and Mineral oil.

Another embodiment comprises cyclosporin A, Empilan KM-50®, and Mineral oil.

Another embodiment comprises cyclosporin A, Empilan L-23®, and Mineral oil.

Another embodiment comprises cyclosporin A, Ethylan 25-3®, and Mineral oil.

Another embodiment comprises cyclosporin A, Ethylan 1204®, and Mineral oil.

Another embodiment comprises cyclosporin A, Ethylan DA-4®, and Mineral oil.

Another embodiment comprises cyclosporin A, Ethylan LA-230®, and Mineral oil.

Another embodiment comprises cyclosporin A, Ethylan SN®, and Mineral oil.

Another embodiment comprises cyclosporin A, Ethylan TD-60®, and Mineral oil.

Another embodiment comprises cyclosporin A, Ethylan TD-100®, and Mineral oil.

Another embodiment comprises cyclosporin A, Ethylan TD-1407®, and Mineral oil.

Another embodiment comprises cyclosporin A, Eumulgin B1®, and Mineral oil.

Another embodiment comprises cyclosporin A, Eumulgin B2®, and Mineral oil.

Another embodiment comprises cyclosporin A, Eumulgin B3®, and Mineral oil.

Another embodiment comprises cyclosporin A, Eumulgin O-10®, and Mineral oil.

Another embodiment comprises cyclosporin A, Flo Mo 80/20®, and Mineral oil.

Another embodiment comprises cyclosporin A, Flo Mo Low Foam®, and Mineral oil.

Another embodiment comprises cyclosporin A, Forlan C-24®, and Mineral oil.

Another embodiment comprises cyclosporin A, Genapol 1454®, and Mineral oil.

Another embodiment comprises cyclosporin A, Genapol BA-020®, and Mineral oil.

Another embodiment comprises cyclosporin A, Genapol BA-040®, and Mineral oil.

Another embodiment comprises cyclosporin A, Genapol C-100®, and Mineral oil.

Another embodiment comprises cyclosporin A, Genapol DA 060®, and Mineral oil.

Another embodiment comprises cyclosporin A, Genapol HS 020®, and Mineral oil.

Another embodiment comprises cyclosporin A, Genapol HS 200®, and Mineral oil.

Another embodiment comprises cyclosporin A, Genapol ID-040®, and Mineral oil.

Another embodiment comprises cyclosporin A, Genapol ID-060®, and Mineral oil.

Another embodiment comprises cyclosporin A, Genapol ID-090®, and Mineral oil.

Another embodiment comprises cyclosporin A, Genapol LA 010®, and Mineral oil.

Another embodiment comprises cyclosporin A, Genapol LA 020®, and Mineral oil.

Another embodiment comprises cyclosporin A, Genapol LA 030®, and Mineral oil.

Another embodiment comprises cyclosporin A, Genapol LA 040®, and Mineral oil.

Another embodiment comprises cyclosporin A, Genapol LA 050®, and Mineral oil.

Another embodiment comprises cyclosporin A, Genapol LA 060®, and Mineral oil.

Another embodiment comprises cyclosporin A, Genapol LA 070®, and Mineral oil.

Another embodiment comprises cyclosporin A, Genapol LA 070S®, and Mineral oil.

Another embodiment comprises cyclosporin A, Genapol LA 230®, and Mineral oil.

Another embodiment comprises cyclosporin A, Genapol O 020®, and Mineral oil.

Another embodiment comprises cyclosporin A, Genapol O 050®, and Mineral oil.

Another embodiment comprises cyclosporin A, Genapol O 100®, and Mineral oil.

Another embodiment comprises cyclosporin A, Genapol O 200®, and Mineral oil.

Another embodiment comprises cyclosporin A, Genapol SA 030®, and Mineral oil.

Another embodiment comprises cyclosporin A, Genapol SA 120®, and Mineral oil.

Another embodiment comprises cyclosporin A, Genapol T-020®, and Mineral oil.

Another embodiment comprises cyclosporin A, Genapol UD-030®, and Mineral oil.

Another embodiment comprises cyclosporin A, Genapol UD-050®, and Mineral oil.

Another embodiment comprises cyclosporin A, Genapol UD-070®, and Mineral oil.

Another embodiment comprises cyclosporin A, Genapol UD-079®, and Mineral oil.

Another embodiment comprises cyclosporin A, Genapol UD-080®, and Mineral oil.

Another embodiment comprises cyclosporin A, Genapol UD-110®, and Mineral oil.

Another embodiment comprises cyclosporin A, Genapol X 030®, and Mineral oil.

Another embodiment comprises cyclosporin A, Genapol X 050®, and Mineral oil.

Another embodiment comprises cyclosporin A, Genapol X 060®, and Mineral oil.

Another embodiment comprises cyclosporin A, Genapol X 070®, and Mineral oil.

Another embodiment comprises cyclosporin A, Genapol X 080®, and Mineral oil.

Another embodiment comprises cyclosporin A, Genapol X 100®, and Mineral oil.

Another embodiment comprises cyclosporin A, Genapol X159®, and Mineral oil.

Another embodiment comprises cyclosporin A, Generol 122 E5®, and Mineral oil.

Another embodiment comprises cyclosporin A, Generol 122 E25®, and Mineral oil.

Another embodiment comprises cyclosporin A, Hostacerin T-3®, and Mineral oil.

Another embodiment comprises cyclosporin A, Iconol LF 110®, and Mineral oil.

Another embodiment comprises cyclosporin A, Incropol CS-20®, and Mineral oil.

Another embodiment comprises cyclosporin A, Lexemul CS-20®, and Mineral oil.

Another embodiment comprises cyclosporin A, Liponic EG-1®, and Mineral oil.

Another embodiment comprises cyclosporin A, Lipowax D®, and Mineral oil.

Another embodiment comprises cyclosporin A, Lipowax G®, and Mineral oil.

Another embodiment comprises cyclosporin A, Lipowax NI®, and Mineral oil.

Another embodiment comprises cyclosporin A, Lipowax P®, and Mineral oil.

Another embodiment comprises cyclosporin A, Lipowax P-31®, and Mineral oil.

Another embodiment comprises cyclosporin A, Lipowax PR®, and Mineral oil.

Another embodiment comprises cyclosporin A, Lumulse CS-20®, and Mineral oil.

Another embodiment comprises cyclosporin A, Macol CSA-20®, and Mineral oil.

Another embodiment comprises cyclosporin A, Marlox B 24/50®, and Mineral oil.

Another embodiment comprises cyclosporin A, Mazawet 77®, and Mineral oil.

Another embodiment comprises cyclosporin A, Norfox 1713®, and Mineral oil.

Another embodiment comprises cyclosporin A, Norfox 2579®, and Mineral oil.

Another embodiment comprises cyclosporin A, Norfox Lo Foam®, and Mineral oil.

Another embodiment comprises cyclosporin A, Promulgen D®, and Mineral oil.

Another embodiment comprises cyclosporin A, Promulgen G®, and Mineral oil.

Another embodiment comprises cyclosporin A, Renex 30®, and Mineral oil.

Another embodiment comprises cyclosporin A, Renex 36®, and Mineral oil.

Another embodiment comprises cyclosporin A, Rhodasurf A 24®, and Mineral oil.

Another embodiment comprises cyclosporin A, Rhodasurf AAE-10®, and Mineral oil.

Another embodiment comprises cyclosporin A, Rhodasurf BEH-25®, and Mineral oil.

Another embodiment comprises cyclosporin A, Rhodasurf BEH-40®, and Mineral oil.

Another embodiment comprises cyclosporin A, Rhodasurf DA 530®, and Mineral oil.

Another embodiment comprises cyclosporin A, Rhodasurf DA 630®, and Mineral oil.

Another embodiment comprises cyclosporin A, Rhodasurf DA 639®, and Mineral oil.

Another embodiment comprises cyclosporin A, Rhodasurf LAN-23®, and Mineral oil.

Another embodiment comprises cyclosporin A, Rhodasurf ON-870®, and Mineral oil.

Another embodiment comprises cyclosporin A, Rhodasurf ON-877®, and Mineral oil.

Another embodiment comprises cyclosporin A, Rhodasurf TB-970 FLK®, and Mineral oil.

Another embodiment comprises cyclosporin A, Ritacet-20®, and Mineral oil.

Another embodiment comprises cyclosporin A, Ritachol 1000®, and Mineral oil.

Another embodiment comprises cyclosporin A, Ritachol 2000®, and Mineral oil.

Another embodiment comprises cyclosporin A, Ritachol 5000®, and Mineral oil.

Another embodiment comprises cyclosporin A, Ritox 35®, and Mineral oil.

Another embodiment comprises cyclosporin A, Surfonic DA-4®, and Mineral oil.

Another embodiment comprises cyclosporin A, Surfonic DA-6®, and Mineral oil.

Another embodiment comprises cyclosporin A, Surfonic L46-7®, and Mineral oil.

Another embodiment comprises cyclosporin A, Surfonic POA®, and Mineral oil.

Another embodiment comprises cyclosporin A, Synthrapol KB®, and Mineral oil.

Another embodiment comprises cyclosporin A, Teginacid®, and Mineral oil.

Another embodiment comprises cyclosporin A, Teginacid C®, and Mineral oil.

Another embodiment comprises cyclosporin A, Tegotens EC 11®, and Mineral oil.

Another embodiment comprises cyclosporin A, Tinegal NA®, and Mineral oil.

Another embodiment comprises cyclosporin A, Tomadol 400®, and Mineral oil.

Another embodiment comprises cyclosporin A, Tomadol 600®, and Mineral oil.

Another embodiment comprises cyclosporin A, Tomadol 900®, and Mineral oil.

Another embodiment comprises cyclosporin A, Uniperol O®, and Mineral oil.

Another embodiment comprises cyclosporin A, Witconol SN Series®, and Mineral oil.

Another embodiment comprises cyclosporin A, an Ethoxylated Alkylphenol, and Mineral oil.

Another embodiment comprises cyclosporin A, Antarox LF-222®, and Mineral oil.

Another embodiment comprises cyclosporin A, Atlox 775®, and Mineral oil.

Another embodiment comprises cyclosporin A, Caloxylate N-9®, and Mineral oil.

Another embodiment comprises cyclosporin A, Canasol NF-1000®, and Mineral oil.

Another embodiment comprises cyclosporin A, Canasol NF-3000®, and Mineral oil.

Another embodiment comprises cyclosporin A, Canasol NF-3070®, and Mineral oil.

Another embodiment comprises cyclosporin A, Canasol OF 1670®, and Mineral oil.

Another embodiment comprises cyclosporin A, Canasol OF 2570®, and Mineral oil.

Another embodiment comprises cyclosporin A, Canasol OF 4070®, and Mineral oil.

Another embodiment comprises cyclosporin A, Chemax DNP-8®, and Mineral oil.

Another embodiment comprises cyclosporin A, Chemax DNP-18®, and Mineral oil.

Another embodiment comprises cyclosporin A, Chemax DNP-150/50®, and Mineral oil.

Another embodiment comprises cyclosporin A, DeSonic 1.5N®, and Mineral oil.

Another embodiment comprises cyclosporin A, DeSonic 4N®, and Mineral oil.

Another embodiment comprises cyclosporin A, DeSonic 5N®, and Mineral oil.

Another embodiment comprises cyclosporin A, DeSonic 6D®, and Mineral oil.

Another embodiment comprises cyclosporin A, DeSonic 6N®, and Mineral oil.

Another embodiment comprises cyclosporin A, DeSonic 7N®, and Mineral oil.

Another embodiment comprises cyclosporin A, DeSonic 9N®, and Mineral oil.

Another embodiment comprises cyclosporin A, DeSonic 10D®, and Mineral oil.

Another embodiment comprises cyclosporin A, DeSonic 11N®, and Mineral oil.

Another embodiment comprises cyclosporin A, DeSonic 12N®, and Mineral oil.

Another embodiment comprises cyclosporin A, DeSonic 15N®, and Mineral oil.

Another embodiment comprises cyclosporin A, DeSonic 20N®, and Mineral oil.

Another embodiment comprises cyclosporin A, Eccoscour RC®, and Mineral oil.

Another embodiment comprises cyclosporin A, Eccoterge EO-100®, and Mineral oil.

Another embodiment comprises cyclosporin A, Emulsifier 632/90%®, and Mineral oil.

Another embodiment comprises cyclosporin A, Geronol AG-821®, and Mineral oil.

Another embodiment comprises cyclosporin A, Gradonic N-95®, and Mineral oil.

Another embodiment comprises cyclosporin A, Hetoxide NP-4®, and Mineral oil.

Another embodiment comprises cyclosporin A, Hetoxide NP-30®, and Mineral oil.

Another embodiment comprises cyclosporin A, Hostapal N-100®, and Mineral oil.

Another embodiment comprises cyclosporin A, Hostapal N-110®, and Mineral oil.

Another embodiment comprises cyclosporin A, Igepal CTA-639W®, and Mineral oil.

Another embodiment comprises cyclosporin A, Igepal DAP-9®, and Mineral oil.

Another embodiment comprises cyclosporin A, Igepal OD-410®, and Mineral oil.

Another embodiment comprises cyclosporin A, Igepal SS-837®, and Mineral oil.

Another embodiment comprises cyclosporin A, Lipocol NP-9 USP®, and Mineral oil.

Another embodiment comprises cyclosporin A, Macol DNP-10®, and Mineral oil.

Another embodiment comprises cyclosporin A, Marlophen NP 5®, and Mineral oil.

Another embodiment comprises cyclosporin A, Marlophen P 1®, and Mineral oil.

Another embodiment comprises cyclosporin A, Surfonic NB®, and Mineral oil.

Another embodiment comprises cyclosporin A, Surfonic OPB-307®, and Mineral oil.

Another embodiment comprises cyclosporin A, Surfonic OPB-407®, and Mineral oil.

Another embodiment comprises cyclosporin A, Syn Fac 334®, and Mineral oil.

Another embodiment comprises cyclosporin A, Syn Fac 8216®, and Mineral oil.

Another embodiment comprises cyclosporin A, Triton N-57®, and Mineral oil.

Another embodiment comprises cyclosporin A, Trycol 6956®, and Mineral oil.

Another embodiment comprises cyclosporin A, Trycol 6961®, and Mineral oil.

Another embodiment comprises cyclosporin A, Trycol 6964®, and Mineral oil.

Another embodiment comprises cyclosporin A, Trycol 6969®, and Mineral oil.

Another embodiment comprises cyclosporin A, Trycol 6974®, and Mineral oil.

Another embodiment comprises cyclosporin A, Witbreak DRB-127®, and Mineral oil.

Another embodiment comprises cyclosporin A, Witbreak DRB-127®, and Mineral oil.

Another embodiment comprises cyclosporin A, Witconol NP Series®, and Mineral oil.

Another embodiment comprises cyclosporin A, an Ethoxylated Aryl Phenol, and Mineral oil.

Another embodiment comprises cyclosporin A, Soprophor BSU®, and Mineral oil.

Another embodiment comprises cyclosporin A, Soprophor CY/8®, and Mineral oil.

Another embodiment comprises cyclosporin A, Soprophor S/25®, and Mineral oil.

Another embodiment comprises cyclosporin A, Witconol NIO®, and Mineral oil.

Another embodiment comprises cyclosporin A, Witconol NIW®, and Mineral oil.

Another embodiment comprises cyclosporin A, Witconol S-100®, and Mineral oil.

Another embodiment comprises cyclosporin A, an Ethoxylated Fatty Acid, and Mineral oil.

Another embodiment comprises cyclosporin A, Aldo PGHMS®, and Mineral oil.

Another embodiment comprises cyclosporin A, Alkamuls TO-15/HR®, and Mineral oil.

Another embodiment comprises cyclosporin A, Armotan AL-69-66®, and Mineral oil.

Another embodiment comprises cyclosporin A, Cerasynt 840®, and Mineral oil.

Another embodiment comprises cyclosporin A, Cerasynt 945®, and Mineral oil.

Another embodiment comprises cyclosporin A, Crystal Inhibitor No. 5®, and Mineral oil.

Another embodiment comprises cyclosporin A, DeThox Acid L-9®, and Mineral oil.

Another embodiment comprises cyclosporin A, DeThox Acid S-8®, and Mineral oil.

Another embodiment comprises cyclosporin A, Ethofat 242/25®, and Mineral oil.

Another embodiment comprises cyclosporin A, Hydropalat 65®, and Mineral oil.

Another embodiment comprises cyclosporin A, Lipo EGMS®, and Mineral oil.

Another embodiment comprises cyclosporin A, Lipopeg 2 DL®, and Mineral oil.

Another embodiment comprises cyclosporin A, Lipopeg 4 DL®, and Mineral oil.

Another embodiment comprises cyclosporin A, Lipopeg 4-L®, and Mineral oil.

Another embodiment comprises cyclosporin A, Lipopeg 39-S®, and Mineral oil.

Another embodiment comprises cyclosporin A, Lipopeg 4-S®, and Mineral oil.

Another embodiment comprises cyclosporin A, Lipopeg 10-S®, and Mineral oil.

Another embodiment comprises cyclosporin A, Lipopeg 100-S®, and Mineral oil.

Another embodiment comprises cyclosporin A, Lipopeg 6000 DS®, and Mineral oil.

Another embodiment comprises cyclosporin A, Lumulse 40-L®, and Mineral oil.

Another embodiment comprises cyclosporin A, Lumulse 40-S®, and Mineral oil.

Another embodiment comprises cyclosporin A, Lumulse 42-L®, and Mineral oil.

Another embodiment comprises cyclosporin A, Lumulse 42-S®, and Mineral oil.

Another embodiment comprises cyclosporin A, Lumulse 100-S®, and Mineral oil.

Another embodiment comprises cyclosporin A, Lumulse 602-S®, and Mineral oil.

Another embodiment comprises cyclosporin A, Magrabar PGE-20-0®, and Mineral oil.

Another embodiment comprises cyclosporin A, Magrabar PGE-20L®, and Mineral oil.

Another embodiment comprises cyclosporin A, Magrabar PGE-20T®, and Mineral oil.

Another embodiment comprises cyclosporin A, Magrabar PGE-22-0®, and Mineral oil.

Another embodiment comprises cyclosporin A, Magrabar PGE-22L®, and Mineral oil.

Another embodiment comprises cyclosporin A, Magrabar PGE-22T®, and Mineral oil.

Another embodiment comprises cyclosporin A, Magrabar PGE-40-0®, and Mineral oil.

Another embodiment comprises cyclosporin A, Magrabar PGE-40L®, and Mineral oil.

Another embodiment comprises cyclosporin A, Magrabar PGE-40T®, and Mineral oil.

Another embodiment comprises cyclosporin A, Magrabar PGE-42-0®, and Mineral oil.

Another embodiment comprises cyclosporin A, Magrabar PGE-42L®, and Mineral oil.

Another embodiment comprises cyclosporin A, Magrabar PGE-42T®, and Mineral oil.

Another embodiment comprises cyclosporin A, Magrabar PGE-60-0®, and Mineral oil.

Another embodiment comprises cyclosporin A, Magrabar PGE-60L®, and Mineral oil.

Another embodiment comprises cyclosporin A, Magrabar PGE-60T®, and Mineral oil.

Another embodiment comprises cyclosporin A, Magrabar PGE-62-0®, and Mineral oil.

Another embodiment comprises cyclosporin A, Magrabar PGE-62L®, and Mineral oil.

Another embodiment comprises cyclosporin A, Magrabar PGE-62T®, and Mineral oil.

Another embodiment comprises cyclosporin A, Mapeg S-40K®, and Mineral oil.

Another embodiment comprises cyclosporin A, Marlowet OTS®, and Mineral oil.

Another embodiment comprises cyclosporin A, Naturechem PGR®, and Mineral oil.

Another embodiment comprises cyclosporin A, PG No. 4®, and Mineral oil.

Another embodiment comprises cyclosporin A, Renex 20®, and Mineral oil.

Another embodiment comprises cyclosporin A, Ritox 52®, and Mineral oil.

Another embodiment comprises cyclosporin A, Ritox 53®, and Mineral oil.

Another embodiment comprises cyclosporin A, Ritox 59®, and Mineral oil.

Another embodiment comprises cyclosporin A, Surfax 8916/A®, and Mineral oil.

Another embodiment comprises cyclosporin A, Tego Acid S 40 P®, and Mineral oil.

Another embodiment comprises cyclosporin A, Tego Acid S 100 P®, and Mineral oil.

Another embodiment comprises cyclosporin A, Tween 20®, and Mineral oil.

Another embodiment comprises cyclosporin A, Volpo 131®, and Mineral oil.

Another embodiment comprises cyclosporin A, an Ethoxylated Fatty Ester or Oil, and Mineral oil.

Another embodiment comprises cyclosporin A, Acconon 6-C10®, and Mineral oil.

Another embodiment comprises cyclosporin A, Acconon CC-6®, and Mineral oil.

Another embodiment comprises cyclosporin A, Acconon CO-7®, and Mineral oil.

Another embodiment comprises cyclosporin A, Aldosperse 40/60 FG®, and Mineral oil.

Another embodiment comprises cyclosporin A, Aldosperse ML-23®, and Mineral oil.

Another embodiment comprises cyclosporin A, Aldosperse MS-20 FG®, and Mineral oil.

Another embodiment comprises cyclosporin A, Alkamuls EL-620®, and Mineral oil.

Another embodiment comprises cyclosporin A, Alkamuls EL-719®, and Mineral oil.

Another embodiment comprises cyclosporin A, Alkamuls EL-985®, and Mineral oil.

Another embodiment comprises cyclosporin A, Arlatone G®, and Mineral oil.

Another embodiment comprises cyclosporin A, Arlatone T®, and Mineral oil.

Another embodiment comprises cyclosporin A, Atlas G-1045A®, and Mineral oil.

Another embodiment comprises cyclosporin A, Atlas G-1086®, and Mineral oil.

Another embodiment comprises cyclosporin A, Atlas G-1087®, and Mineral oil.

Another embodiment comprises cyclosporin A, Atlas G-1089®, and Mineral oil.

Another embodiment comprises cyclosporin A, Atlas G-1096®, and Mineral oil.

Another embodiment comprises cyclosporin A, Atlas G-1292®, and Mineral oil.

Another embodiment comprises cyclosporin A, Atlas G-1293®, and Mineral oil.

Another embodiment comprises cyclosporin A, Atlas G-1300®, and Mineral oil.

Another embodiment comprises cyclosporin A, Atlas G-7076®, and Mineral oil.

Another embodiment comprises cyclosporin A, Capmul EMG®, and Mineral oil.

Another embodiment comprises cyclosporin A, Chemonic CO-40®, and Mineral oil.

Another embodiment comprises cyclosporin A, Chemonic LI-3®, and Mineral oil.

Another embodiment comprises cyclosporin A, Chemonic LI-7®, and Mineral oil.

Another embodiment comprises cyclosporin A, Cirrasol GM®, and Mineral oil.

Another embodiment comprises cyclosporin A, Cremophor CO 40®, and Mineral oil.

Another embodiment comprises cyclosporin A, Cremophor CO 410®, and Mineral oil.

Another embodiment comprises cyclosporin A, Cremophor EL®, and Mineral oil.

Another embodiment comprises cyclosporin A, Cremophor GC7®, and Mineral oil.

Another embodiment comprises cyclosporin A, Cremophor RH-40®, and Mineral oil.

Another embodiment comprises cyclosporin A, Crovol A-40®, and Mineral oil.

Another embodiment comprises cyclosporin A, Crovol A-70®, and Mineral oil.

Another embodiment comprises cyclosporin A, Crovol M-70®, and Mineral oil.

Another embodiment comprises cyclosporin A, Crovol PK-70®, and Mineral oil.

Another embodiment comprises cyclosporin A, Cutina E-24®, and Mineral oil.

Another embodiment comprises cyclosporin A, Dacospin 12-R®, and Mineral oil.

Another embodiment comprises cyclosporin A, Dehymuls HRE-7®, and Mineral oil.

Another embodiment comprises cyclosporin A, DeSonic 30C®, and Mineral oil.

Another embodiment comprises cyclosporin A, DeSonic 36C®, and Mineral oil.

Another embodiment comprises cyclosporin A, DeSonic 40C®, and Mineral oil.

Another embodiment comprises cyclosporin A, Durfax 60®, and Mineral oil.

Another embodiment comprises cyclosporin A, Durfax 65®, and Mineral oil.

Another embodiment comprises cyclosporin A, Durfax 80®, and Mineral oil.

Another embodiment comprises cyclosporin A, Durfax EOM®, and Mineral oil.

Another embodiment comprises cyclosporin A, Eccoterge NF-2®, and Mineral oil.

Another embodiment comprises cyclosporin A, Emulpon CO-360®, and Mineral oil.

Another embodiment comprises cyclosporin A, Emulpon CO-550®, and Mineral oil.

Another embodiment comprises cyclosporin A, Emulsogen EL®, and Mineral oil.

Another embodiment comprises cyclosporin A, Emulsogen HCO 040®, and Mineral oil.

Another embodiment comprises cyclosporin A, Emulsogen HCO 060®, and Mineral oil.

Another embodiment comprises cyclosporin A, Emulsynt 1055®, and Mineral oil.

Another embodiment comprises cyclosporin A, Ethox 3095®, and Mineral oil.

Another embodiment comprises cyclosporin A, Eumulgin RO-40®, and Mineral oil.

Another embodiment comprises cyclosporin A, Genapol G-260®, and Mineral oil.

Another embodiment comprises cyclosporin A, Glycosperse L-20®, and Mineral oil.

Another embodiment comprises cyclosporin A, Glycosperse O-5®, and Mineral oil.

Another embodiment comprises cyclosporin A, Glycosperse O-20®, and Mineral oil.

Another embodiment comprises cyclosporin A, Glycosperse O-20 FG®, and Mineral oil.

Another embodiment comprises cyclosporin A, Glycosperse S-20®, and Mineral oil.

Another embodiment comprises cyclosporin A, Glycosperse S-20 FG®, and Mineral oil.

Another embodiment comprises cyclosporin A, Glycosperse TS-20®, and Mineral oil.

Another embodiment comprises cyclosporin A, Glycosperse TS-20 FG®, and Mineral oil.

Another embodiment comprises cyclosporin A, Hetan SL®, and Mineral oil.

Another embodiment comprises cyclosporin A, Hetan SO®, and Mineral oil.

Another embodiment comprises cyclosporin A, Hetan SS®, and Mineral oil.

Another embodiment comprises cyclosporin A, Hetoxide C-2®, and Mineral oil.

Another embodiment comprises cyclosporin A, Hetoxide C-9®, and Mineral oil.

Another embodiment comprises cyclosporin A, Hetoxide C-15®, and Mineral oil.

Another embodiment comprises cyclosporin A, Hetoxide C-25®, and Mineral oil.

Another embodiment comprises cyclosporin A, Hetoxide C-40®, and Mineral oil.

Another embodiment comprises cyclosporin A, Hetoxide C-200®, and Mineral oil.

Another embodiment comprises cyclosporin A, Hetoxide C-200-50%®, and Mineral oil.

Another embodiment comprises cyclosporin A, Hetoxide GC-30®, and Mineral oil.

Another embodiment comprises cyclosporin A, Hetoxide HC-60®, and Mineral oil.

Another embodiment comprises cyclosporin A, Ice No. 2®, and Mineral oil.

Another embodiment comprises cyclosporin A, Incrocas 30/40®, and Mineral oil.

Another embodiment comprises cyclosporin A, Lexol EC®, and Mineral oil.

Another embodiment comprises cyclosporin A, Lexol EO®, and Mineral oil.

Another embodiment comprises cyclosporin A, Lipocol HCO-40®, and Mineral oil.

Another embodiment comprises cyclosporin A, Lipocol HCO-60®, and Mineral oil.

Another embodiment comprises cyclosporin A, Lipocol O-3 Special®, and Mineral oil.

Another embodiment comprises cyclosporin A, Lipopeg 2-L®, and Mineral oil.

Another embodiment comprises cyclosporin A, Lipopeg 4-DO®, and Mineral oil.

Another embodiment comprises cyclosporin A, Lipopeg 4-DS®, and Mineral oil.

Another embodiment comprises cyclosporin A, Lipovol GTB®, and Mineral oil.

Another embodiment comprises cyclosporin A, Lonzest SML-20®, and Mineral oil.

Another embodiment comprises cyclosporin A, Lonzest SMO-20®, and Mineral oil.

Another embodiment comprises cyclosporin A, Lonzest SMS-20®, and Mineral oil.

Another embodiment comprises cyclosporin A, Lonzest STO-20®, and Mineral oil.

Another embodiment comprises cyclosporin A, Lonzest STS-20®, and Mineral oil.

Another embodiment comprises cyclosporin A, Lumulse GR-40®, and Mineral oil.

Another embodiment comprises cyclosporin A, Lumulse GRH-40®, and Mineral oil.

Another embodiment comprises cyclosporin A, Lumulse POE (7) GML®, and Mineral oil.

Another embodiment comprises cyclosporin A, Lumulse POE (12) Glyc®, and Mineral oil.

Another embodiment comprises cyclosporin A, Lumulse POE (40) MS KP®, and Mineral oil.

Another embodiment comprises cyclosporin A, Marlowet 4750®, and Mineral oil.

Another embodiment comprises cyclosporin A, Marlowet LVS®, and Mineral oil.

Another embodiment comprises cyclosporin A, Marlowet R 11®, and Mineral oil.

Another embodiment comprises cyclosporin A, Marlowet R 40®, and Mineral oil.

Another embodiment comprises cyclosporin A, Mazol 80 MGK®, and Mineral oil.

Another embodiment comprises cyclosporin A, Nonionic Emulsifier T-9®, and Mineral oil.

Another embodiment comprises cyclosporin A, Oronal LCG®, and Mineral oil.

Another embodiment comprises cyclosporin A, Polyderm PPI-CO-200®, and Mineral oil.

Another embodiment comprises cyclosporin A, Polyderm PPI-CO-40®, and Mineral oil.

Another embodiment comprises cyclosporin A, Rewoderm LI 520-70®, and Mineral oil.

Another embodiment comprises cyclosporin A, Ritapeg 150 DS®, and Mineral oil.

Another embodiment comprises cyclosporin A, Softigen 767®, and Mineral oil.

Another embodiment comprises cyclosporin A, Surfactol 318®, and Mineral oil.

Another embodiment comprises cyclosporin A, Surfactol 365®, and Mineral oil.

Another embodiment comprises cyclosporin A, Syn Lube 107®, and Mineral oil.

Another embodiment comprises cyclosporin A, Syn Lube 728®, and Mineral oil.

Another embodiment comprises cyclosporin A, Syn Lube 1632H®, and Mineral oil.

Another embodiment comprises cyclosporin A, Syn Lube 6277-A®, and Mineral oil.

Another embodiment comprises cyclosporin A, T-Det C-20®, and Mineral oil.

Another embodiment comprises cyclosporin A, T-Det C-40®, and Mineral oil.

Another embodiment comprises cyclosporin A, Tally 100 Plus®, and Mineral oil.

Another embodiment comprises cyclosporin A, Uniperol EL®, and Mineral oil.

Another embodiment comprises cyclosporin A, a Fatty Ester, and Mineral oil.

Another embodiment comprises cyclosporin A, Actralube-Syn 147®, and Mineral oil.

Another embodiment comprises cyclosporin A, Atlas G-1556®, and Mineral oil.

Another embodiment comprises cyclosporin A, Atlas G-1564®, and Mineral oil.

Another embodiment comprises cyclosporin A, Atlasol Base Oil S®, and Mineral oil.

Another embodiment comprises cyclosporin A, Base ML®, and Mineral oil.

Another embodiment comprises cyclosporin A, Base MT®, and Mineral oil.

Another embodiment comprises cyclosporin A, Cerasynt 303®, and Mineral oil.

Another embodiment comprises cyclosporin A, Dermol 1012®, and Mineral oil.

Another embodiment comprises cyclosporin A, Kemester 4000®, and Mineral oil.

Another embodiment comprises cyclosporin A, Lactipol S®, and Mineral oil.

Another embodiment comprises cyclosporin A, Magrabar PGO®, and Mineral oil.

Another embodiment comprises cyclosporin A, Mayco Base BFO®, and Mineral oil.

Another embodiment comprises cyclosporin A, Methyl Linoleate®, and Mineral oil.

Another embodiment comprises cyclosporin A, Pationic 122A®, and Mineral oil.

Another embodiment comprises cyclosporin A, Pationic 138C®, and Mineral oil.

Another embodiment comprises cyclosporin A, Pationic CSL®, and Mineral oil.

Another embodiment comprises cyclosporin A, Pationic ISL®, and Mineral oil.

Another embodiment comprises cyclosporin A, Pationic SBL®, and Mineral oil.

Another embodiment comprises cyclosporin A, Pationic SSL®, and Mineral oil.

Another embodiment comprises cyclosporin A, Ritasol®, and Mineral oil.

Another embodiment comprises cyclosporin A, Tego Alkanol CS 20®, and Mineral oil.

Another embodiment comprises cyclosporin A, Tego Alkanol L23 P®, and Mineral oil.

Another embodiment comprises cyclosporin A, Tego Alkanol S2®, and Mineral oil.

Another embodiment comprises cyclosporin A, Tego Alkanol S20 P®, and Mineral oil.

Another embodiment comprises cyclosporin A, Triemulsifier 600 MS®, and Mineral oil.

Another embodiment comprises cyclosporin A, a Glycerol Esters, and Mineral oil.

Another embodiment comprises cyclosporin A, Agro #9 Wint SBO®, and Mineral oil.

Another embodiment comprises cyclosporin A, Ahcovel Base 700®, and Mineral oil.

Another embodiment comprises cyclosporin A, Aldo HMS FG®, and Mineral oil.

Another embodiment comprises cyclosporin A, Aldo MLD®, and Mineral oil.

Another embodiment comprises cyclosporin A, Aldo MLD FG®, and Mineral oil.

Another embodiment comprises cyclosporin A, Aldo MO FG®, and Mineral oil.

Another embodiment comprises cyclosporin A, Aldo MS®, and Mineral oil.

Another embodiment comprises cyclosporin A, Aldo MS FG®, and Mineral oil.

Another embodiment comprises cyclosporin A, Aldo MS LG FG®, and Mineral oil.

Another embodiment comprises cyclosporin A, Aldo MSD®, and Mineral oil.

Another embodiment comprises cyclosporin A, Aldo MSD FG®, and Mineral oil.

Another embodiment comprises cyclosporin A, Aldosperse O-20 FG®, and Mineral oil.

Another embodiment comprises cyclosporin A, Aldosperse TS-20 FG®, and Mineral oil.

Another embodiment comprises cyclosporin A, Aldosperse TS-40 FG®, and Mineral oil.

Another embodiment comprises cyclosporin A, Arlacel 165®, and Mineral oil.

Another embodiment comprises cyclosporin A, Arlacel 186®, and Mineral oil.

Another embodiment comprises cyclosporin A, Capmul GMO®, and Mineral oil.

Another embodiment comprises cyclosporin A, Capmul GMS®, and Mineral oil.

Another embodiment comprises cyclosporin A, Caprol 3GO®, and Mineral oil.

Another embodiment comprises cyclosporin A, Caprol 3GVS®, and Mineral oil.

Another embodiment comprises cyclosporin A, Caprol 6G2S®, and Mineral oil.

Another embodiment comprises cyclosporin A, Caprol 10G40®, and Mineral oil.

Another embodiment comprises cyclosporin A, Caprol 10G100®, and Mineral oil.

Another embodiment comprises cyclosporin A, Caprol ET®, and Mineral oil.

Another embodiment comprises cyclosporin A, Caprol PGE860®, and Mineral oil.

Another embodiment comprises cyclosporin A, Cerasynt 945®, and Mineral oil.

Another embodiment comprises cyclosporin A, Cerasynt GMS®, and Mineral oil.

Another embodiment comprises cyclosporin A, Cerasynt Q®, and Mineral oil.

Another embodiment comprises cyclosporin A, Cerasynt SD®, and Mineral oil.

Another embodiment comprises cyclosporin A, Cerasynt WM®, and Mineral oil.

Another embodiment comprises cyclosporin A, Chemsperse 14®, and Mineral oil.

Another embodiment comprises cyclosporin A, Cremophor GO-32®, and Mineral oil.

Another embodiment comprises cyclosporin A, Cremophor GS11®, and Mineral oil.

Another embodiment comprises cyclosporin A, Cremophor GS-32®, and Mineral oil.

Another embodiment comprises cyclosporin A, Cutina KD-16®, and Mineral oil.

Another embodiment comprises cyclosporin A, Dehymuls PGPH®, and Mineral oil.

Another embodiment comprises cyclosporin A, Dermol DGDIS®, and Mineral oil.

Another embodiment comprises cyclosporin A, Dermol DGMIS®, and Mineral oil.

Another embodiment comprises cyclosporin A, Dermol G-76®, and Mineral oil.

Another embodiment comprises cyclosporin A, Dermol G-7DI®, and Mineral oil.

Another embodiment comprises cyclosporin A, Dermol NGDI®, and Mineral oil.

Another embodiment comprises cyclosporin A, Dermolan GLH®, and Mineral oil.

Another embodiment comprises cyclosporin A, Drewmulse GMO®, and Mineral oil.

Another embodiment comprises cyclosporin A, Drewpol 3-5-M®, and Mineral oil.

Another embodiment comprises cyclosporin A, Durlac 100 W®, and Mineral oil.

Another embodiment comprises cyclosporin A, Dur-Lo®, and Mineral oil.

Another embodiment comprises cyclosporin A, Dynasan 118®, and Mineral oil.

Another embodiment comprises cyclosporin A, EC-25®, and Mineral oil.

Another embodiment comprises cyclosporin A, EM 40®, and Mineral oil.

Another embodiment comprises cyclosporin A, Emerest 2400®, and Mineral oil.

Another embodiment comprises cyclosporin A, Emerest 2452®, and Mineral oil.

Another embodiment comprises cyclosporin A, Empilan G-26®, and Mineral oil.

Another embodiment comprises cyclosporin A, Genapol TSM®, and Mineral oil.

Another embodiment comprises cyclosporin A, Hostacerin DGI®, and Mineral oil.

Another embodiment comprises cyclosporin A, Hostacerin DGL®, and Mineral oil.

Another embodiment comprises cyclosporin A, Hostacerin DGMS®, and Mineral oil.

Another embodiment comprises cyclosporin A, Hostacerin DGSB®, and Mineral oil.

Another embodiment comprises cyclosporin A, Ice No. 2®, and Mineral oil.

Another embodiment comprises cyclosporin A, Imwitor 742®, and Mineral oil.

Another embodiment comprises cyclosporin A, Imwitor 780 K®, and Mineral oil.

Another embodiment comprises cyclosporin A, Imwitor 960 Flakes®, and Mineral oil.

Another embodiment comprises cyclosporin A, Isolan GI 34®, and Mineral oil.

Another embodiment comprises cyclosporin A, Isolan GO 33®, and Mineral oil.

Another embodiment comprises cyclosporin A, Kemester 1000®, and Mineral oil.

Another embodiment comprises cyclosporin A, Kemester 2000®, and Mineral oil.

Another embodiment comprises cyclosporin A, Kemester 2000®, and Mineral oil.

Another embodiment comprises cyclosporin A, Kemester 6000SE®, and Mineral oil.

Another embodiment comprises cyclosporin A, Lamecreme DGE 18®, and Mineral oil.

Another embodiment comprises cyclosporin A, Lexemul 515®, and Mineral oil.

Another embodiment comprises cyclosporin A, Lexemul 561®, and Mineral oil.

Another embodiment comprises cyclosporin A, Lexemul AR®, and Mineral oil.

Another embodiment comprises cyclosporin A, Lexemul AS®, and Mineral oil.

Another embodiment comprises cyclosporin A, Lexemul GDL®, and Mineral oil.

Another embodiment comprises cyclosporin A, Lexemul T®, and Mineral oil.

Another embodiment comprises cyclosporin A, Lipomulse 165®, and Mineral oil.

Another embodiment comprises cyclosporin A, Lumulse GML K®, and Mineral oil.

Another embodiment comprises cyclosporin A, Lumulse GMO K®, and Mineral oil.

Another embodiment comprises cyclosporin A, Lumulse GMR K®, and Mineral oil.

Another embodiment comprises cyclosporin A, Lumulse GMT K®, and Mineral oil.

Another embodiment comprises cyclosporin A, Magrabar GMC®, and Mineral oil.

Another embodiment comprises cyclosporin A, Magrabar GMO-CK®, and Mineral oil.

Another embodiment comprises cyclosporin A, Magrabar MDG-5050®, and Mineral oil.

Another embodiment comprises cyclosporin A, Magrabar PGO-315®, and Mineral oil.

Another embodiment comprises cyclosporin A, Magrabar PGO-1010®, and Mineral oil.

Another embodiment comprises cyclosporin A, Mazol 300K®, and Mineral oil.

Another embodiment comprises cyclosporin A, Mazol GMO-K®, and Mineral oil.

Another embodiment comprises cyclosporin A, Mazol GMS-K®, and Mineral oil.

Another embodiment comprises cyclosporin A, Mazol PG031 -K®, and Mineral oil.

Another embodiment comprises cyclosporin A, Miglyol 812®, and Mineral oil.

Another embodiment comprises cyclosporin A, Norfox 165C®, and Mineral oil.

Another embodiment comprises cyclosporin A, Schercemol GMIS®, and Mineral oil.

Another embodiment comprises cyclosporin A, Tegin®, and Mineral oil.

Another embodiment comprises cyclosporin A, Tegin 4100 Pellets®, and Mineral oil.

Another embodiment comprises cyclosporin A, Tegin M Pellets®, and Mineral oil.

Another embodiment comprises cyclosporin A, Tegin OV®, and Mineral oil.

Another embodiment comprises cyclosporin A, Teginacid H®, and Mineral oil.

Another embodiment comprises cyclosporin A, Tego Cosmo P813®, and Mineral oil.

Another embodiment comprises cyclosporin A, Wickenol 535®, and Mineral oil.

Another embodiment comprises cyclosporin A, Witconol 14®, and Mineral oil.

Another embodiment comprises cyclosporin A, Witconol 14®, and Mineral oil.

Another embodiment comprises cyclosporin A, Witconol 14F®, and Mineral oil.

Another embodiment comprises cyclosporin A, Witconol 18L®, and Mineral oil.

Another embodiment comprises cyclosporin A, Witconol GOT®, and Mineral oil.

Another embodiment comprises cyclosporin A, Witconol MST®, and Mineral oil.

Another embodiment comprises cyclosporin A, Witconol RHT®, and Mineral oil.

Another embodiment comprises cyclosporin A, a Glycol Ester, and Mineral oil.

Another embodiment comprises cyclosporin A, Alkamuls 600 DO®, and Mineral oil.

Another embodiment comprises cyclosporin A, Alkamuls SEG®, and Mineral oil.

Another embodiment comprises cyclosporin A, Atlas EM-2®, and Mineral oil.

Another embodiment comprises cyclosporin A, Cerasynt IP®, and Mineral oil.

Another embodiment comprises cyclosporin A, Cerasynt M®, and Mineral oil.

Another embodiment comprises cyclosporin A, Cerasynt MN®, and Mineral oil.

Another embodiment comprises cyclosporin A, Cerasynt PA®, and Mineral oil.

Another embodiment comprises cyclosporin A, Chemsperse EGDS®, and Mineral oil.

Another embodiment comprises cyclosporin A, Chemsperse EGMS®, and Mineral oil.

Another embodiment comprises cyclosporin A, Colonial Monolaurin®, and Mineral oil.

Another embodiment comprises cyclosporin A, DeMuls SGE-95®, and Mineral oil.

Another embodiment comprises cyclosporin A, Eccoterge 200®, and Mineral oil.

Another embodiment comprises cyclosporin A, Emerest 2380®, and Mineral oil.

Another embodiment comprises cyclosporin A, Ethox 2610®, and Mineral oil.

Another embodiment comprises cyclosporin A, Ethox DO-9®, and Mineral oil.

Another embodiment comprises cyclosporin A, Ethox DO-14®, and Mineral oil.

Another embodiment comprises cyclosporin A, Ethox SO-9®, and Mineral oil.

Another embodiment comprises cyclosporin A, Fizul MD-318®, and Mineral oil.

Another embodiment comprises cyclosporin A, Genapol EGDS-VHP®, and Mineral oil.

Another embodiment comprises cyclosporin A, Genapol TS Powder®, and Mineral oil.

Another embodiment comprises cyclosporin A, Hostacerin WO®, and Mineral oil.

Another embodiment comprises cyclosporin A, Inversol 140®, and Mineral oil.

Another embodiment comprises cyclosporin A, Kemester 104®, and Mineral oil.

Another embodiment comprises cyclosporin A, Kemester 205®, and Mineral oil.

Another embodiment comprises cyclosporin A, Kemester 226®, and Mineral oil.

Another embodiment comprises cyclosporin A, Kemester 5221 SE®, and Mineral oil.

Another embodiment comprises cyclosporin A, Kemester EGDS®, and Mineral oil.

Another embodiment comprises cyclosporin A, Lexemul EGDS®, and Mineral oil.

Another embodiment comprises cyclosporin A, Lexemul EGMS®, and Mineral oil.

Another embodiment comprises cyclosporin A, Lexemul P®, and Mineral oil.

Another embodiment comprises cyclosporin A, Lipo DGLS®, and Mineral oil.

Another embodiment comprises cyclosporin A, Lipo EGDS®, and Mineral oil.

Another embodiment comprises cyclosporin A, Lipo PGMS®, and Mineral oil.

Another embodiment comprises cyclosporin A, Liposorb S-4®, and Mineral oil.

Another embodiment comprises cyclosporin A, Liposorb TO-20®, and Mineral oil.

Another embodiment comprises cyclosporin A, Lumulse PGO®, and Mineral oil.

Another embodiment comprises cyclosporin A, Mackester EGDS®, and Mineral oil.

Another embodiment comprises cyclosporin A, Mackester EGMS®, and Mineral oil.

Another embodiment comprises cyclosporin A, Mackester GSTP®, and Mineral oil.

Another embodiment comprises cyclosporin A, Mackester Series®, and Mineral oil.

Another embodiment comprises cyclosporin A, Magrabar PDG-50®, and Mineral oil.

Another embodiment comprises cyclosporin A, Mapeg 6000 DS®, and Mineral oil.

Another embodiment comprises cyclosporin A, Marlowet 4702®, and Mineral oil.

Another embodiment comprises cyclosporin A, Monalube 305®, and Mineral oil.

Another embodiment comprises cyclosporin A, Monalube 310®, and Mineral oil.

Another embodiment comprises cyclosporin A, Monalube 315®, and Mineral oil.

Another embodiment comprises cyclosporin A, Monalube 320®, and Mineral oil.

Another embodiment comprises cyclosporin A, Monalube 325®, and Mineral oil.

Another embodiment comprises cyclosporin A, Monalube 330®, and Mineral oil.

Another embodiment comprises cyclosporin A, Naturechem PGHS®, and Mineral oil.

Another embodiment comprises cyclosporin A, Polycastorol PLO-840®, and Mineral oil.

Another embodiment comprises cyclosporin A, Polytex 10M®, and Mineral oil.

Another embodiment comprises cyclosporin A, Ritasynt IP®, and Mineral oil.

Another embodiment comprises cyclosporin A, Ross Chem PEG 600 DT®, and Mineral oil.

Another embodiment comprises cyclosporin A, Schercemol PGMS®, and Mineral oil.

Another embodiment comprises cyclosporin A, Sponto H-44C®, and Mineral oil.

Another embodiment comprises cyclosporin A, Tegin G®, and Mineral oil.

Another embodiment comprises cyclosporin A, Witbreak DGE-182®, and Mineral oil.

Another embodiment comprises cyclosporin A, Witbreak DGE-182®, and Mineral oil.

Another embodiment comprises cyclosporin A, Witbreak DRA-21®, and Mineral oil.

Another embodiment comprises cyclosporin A, Witbreak DRA-21®, and Mineral oil.

Another embodiment comprises cyclosporin A, Witbreak DRA-50®, and Mineral oil.

Another embodiment comprises cyclosporin A, Witbreak DRA-50®, and Mineral oil.

Another embodiment comprises cyclosporin A, Witconol F26-46®, and Mineral oil.

Another embodiment comprises cyclosporin A, Witconol H-32®, and Mineral oil.

Another embodiment comprises cyclosporin A, Witconol H-33®, and Mineral oil.

Another embodiment comprises cyclosporin A, Witconol H-35A®, and Mineral oil.

Another embodiment comprises cyclosporin A, Witconol RHP®, and Mineral oil.

Another embodiment comprises cyclosporin A, a Lanolin-based Derivative, and Mineral oil.

Another embodiment comprises cyclosporin A, Amerchol CAB®, and Mineral oil.

Another embodiment comprises cyclosporin A, Amerchol L-101®, and Mineral oil.

Another embodiment comprises cyclosporin A, Amerlate LFA-LO®, and Mineral oil.

Another embodiment comprises cyclosporin A, Amerlate P®, and Mineral oil.

Another embodiment comprises cyclosporin A, Barre Common Degras®, and Mineral oil.

Another embodiment comprises cyclosporin A, Cholesterol NF®, and Mineral oil.

Another embodiment comprises cyclosporin A, Crodalan AWS®, and Mineral oil.

Another embodiment comprises cyclosporin A, Crodalan LA®, and Mineral oil.

Another embodiment comprises cyclosporin A, Emery 1650®, and Mineral oil.

Another embodiment comprises cyclosporin A, Emery 1650®, and Mineral oil.

Another embodiment comprises cyclosporin A, Emery 1740®, and Mineral oil.

Another embodiment comprises cyclosporin A, Emery 1740®, and Mineral oil.

Another embodiment comprises cyclosporin A, Forlan 500®, and Mineral oil.

Another embodiment comprises cyclosporin A, Forlan L®, and Mineral oil.

Another embodiment comprises cyclosporin A, Laneto 50®, and Mineral oil.

Another embodiment comprises cyclosporin A, Laneto 100®, and Mineral oil.

Another embodiment comprises cyclosporin A, Laneto AWS®, and Mineral oil.

Another embodiment comprises cyclosporin A, Lanfrax 1776®, and Mineral oil.

Another embodiment comprises cyclosporin A, Lanfrax 1776®, and Mineral oil.

Another embodiment comprises cyclosporin A, Lanogel 21®, and Mineral oil.

Another embodiment comprises cyclosporin A, Lipolan®, and Mineral oil.

Another embodiment comprises cyclosporin A, Lipolan 31®, and Mineral oil.

Another embodiment comprises cyclosporin A, OHlan®, and Mineral oil.

Another embodiment comprises cyclosporin A, Polychol 5®, and Mineral oil.

Another embodiment comprises cyclosporin A, Polychol 15®, and Mineral oil.

Another embodiment comprises cyclosporin A, Ritacetyl®, and Mineral oil.

Another embodiment comprises cyclosporin A, Ritachol®, and Mineral oil.

Another embodiment comprises cyclosporin A, Ritahydrox®, and Mineral oil.

Another embodiment comprises cyclosporin A, Ritalafa®, and Mineral oil.

Another embodiment comprises cyclosporin A, Ritalan®, and Mineral oil.

Another embodiment comprises cyclosporin A, Ritalan AWS®, and Mineral oil.

Another embodiment comprises cyclosporin A, Ritalan C®, and Mineral oil.

Another embodiment comprises cyclosporin A, Ritawax®, and Mineral oil.

Another embodiment comprises cyclosporin A, Ritawax AEO®, and Mineral oil.

Another embodiment comprises cyclosporin A, Ritawax ALA®, and Mineral oil.

Another embodiment comprises cyclosporin A, Solan/Solan 50/Super Solan®, and Mineral oil.

Another embodiment comprises cyclosporin A, Super Hartolan/Hartolan®, and Mineral oil.

Another embodiment comprises cyclosporin A, Supersat AWS-4®, and Mineral oil.

Another embodiment comprises cyclosporin A, Supersat AWS-24®, and Mineral oil.

Another embodiment comprises cyclosporin A, a Lecithin or Lecithin Derivative, and Mineral oil.

Another embodiment comprises cyclosporin A, Alcolec®, and Mineral oil.

Another embodiment comprises cyclosporin A, Lecithin®, and Mineral oil.

Another embodiment comprises cyclosporin A, Lexin K®, and Mineral oil.

Another embodiment comprises cyclosporin A, Natipide®, and Mineral oil.

Another embodiment comprises cyclosporin A, a Lignin or Lignin Derivative, and Mineral oil.

Another embodiment comprises cyclosporin A, Diwatex XP 9®, and Mineral oil.

Another embodiment comprises cyclosporin A, Dynasperse LCD®, and Mineral oil.

Another embodiment comprises cyclosporin A, Indulin SAL®, and Mineral oil.

Another embodiment comprises cyclosporin A, Indulin W-1®, and Mineral oil.

Another embodiment comprises cyclosporin A, Indulin W-5®, and Mineral oil.

Another embodiment comprises cyclosporin A, Lignosol FTA®, and Mineral oil.

Another embodiment comprises cyclosporin A, Lignosol SFX-65®, and Mineral oil.

Another embodiment comprises cyclosporin A, Marasperse 52 CP®, and Mineral oil.

Another embodiment comprises cyclosporin A, Marasperse AG®, and Mineral oil.

Another embodiment comprises cyclosporin A, Marasperse CBOS-4®, and Mineral oil.

Another embodiment comprises cyclosporin A, Ufoxane 2®, and Mineral oil.

Another embodiment comprises cyclosporin A, a Methyl Ester, and Mineral oil.

Another embodiment comprises cyclosporin A, E.B. Cleaner AK®, and Mineral oil.

Another embodiment comprises cyclosporin A, Oleocal ME-70®, and Mineral oil.

Another embodiment comprises cyclosporin A, Trade Name Company®, and Mineral oil.

Another embodiment comprises cyclosporin A, Methyl Esters (cont'd)®, and Mineral oil.

Another embodiment comprises cyclosporin A, Oleocal ME-92®, and Mineral oil.

Another embodiment comprises cyclosporin A, Oleocal ME-112®, and Mineral oil.

Another embodiment comprises cyclosporin A, Oleocal ME-130®, and Mineral oil.

Another embodiment comprises cyclosporin A, a Monoglyceride or a Derivative thereof, and Mineral oil.

Another embodiment comprises cyclosporin A, Dynacet 211®, and Mineral oil.

Another embodiment comprises cyclosporin A, Hetsorb S-20®, and Mineral oil.

Another embodiment comprises cyclosporin A, Imwitor 191®, and Mineral oil.

Another embodiment comprises cyclosporin A, Imwitor 370®, and Mineral oil.

Another embodiment comprises cyclosporin A, Imwitor 375®, and Mineral oil.

Another embodiment comprises cyclosporin A, Imwitor 900®, and Mineral oil.

Another embodiment comprises cyclosporin A, Imwitor 945®, and Mineral oil.

Another embodiment comprises cyclosporin A, Imwitor 2020®, and Mineral oil.

Another embodiment comprises cyclosporin A, Kemester 5500®, and Mineral oil.

Another embodiment comprises cyclosporin A, Kemester 6000®, and Mineral oil.

Another embodiment comprises cyclosporin A, Magrabar GMC®, and Mineral oil.

Another embodiment comprises cyclosporin A, Magrabar GMO-CK®, and Mineral oil.

Another embodiment comprises cyclosporin A, Magrabar GPC-10®, and Mineral oil.

Another embodiment comprises cyclosporin A, Magrabar MDG-5050®, and Mineral oil.

Another embodiment comprises cyclosporin A, Monalube 335®, and Mineral oil.

Another embodiment comprises cyclosporin A, Monoglycerides Glyceryl Monestearate Archer Daniels Midland Company®, and Mineral oil.

Another embodiment comprises cyclosporin A, Rita GMS®, and Mineral oil.

Another embodiment comprises cyclosporin A, Ritamulse SCG®, and Mineral oil.

Another embodiment comprises cyclosporin A, Softigen 701®, and Mineral oil.

Another embodiment comprises cyclosporin A, Tally 100 Plus®, and Mineral oil.

Another embodiment comprises cyclosporin A, a Polyethylene Glycol, and Mineral oil.

Another embodiment comprises cyclosporin A, Emulgade PL 68/50®, and Mineral oil.

Another embodiment comprises cyclosporin A, Lumulse PEG®, and Mineral oil.

Another embodiment comprises cyclosporin A, Rhodasurf PEG-400®, and Mineral oil.

Another embodiment comprises cyclosporin A, Rhodasurf PEG-600®, and Mineral oil.

Another embodiment comprises cyclosporin A, Witconol PEG-400®, and Mineral oil.

Another embodiment comprises cyclosporin A, a Polymeric Surfactant, and Mineral oil.

Another embodiment comprises cyclosporin A, Acritamer PNC-EG®, and Mineral oil.

Another embodiment comprises cyclosporin A, Ag-Rho DEP-775®, and Mineral oil.

Another embodiment comprises cyclosporin A, APG 325N Glycosides®, and Mineral oil.

Another embodiment comprises cyclosporin A, Aristoflex AVC®, and Mineral oil.

Another embodiment comprises cyclosporin A, Aristoflex HMB®, and Mineral oil.

Another embodiment comprises cyclosporin A, Burco NPS-225®, and Mineral oil.

Another embodiment comprises cyclosporin A, Burco NPS-816®, and Mineral oil.

Another embodiment comprises cyclosporin A, Chemccinate 5603®, and Mineral oil.

Another embodiment comprises cyclosporin A, Cosmedia Guar C-261 N®, and Mineral oil.

Another embodiment comprises cyclosporin A, Gantrez S-95®, and Mineral oil.

Another embodiment comprises cyclosporin A, Glucopon 220 UP®, and Mineral oil.

Another embodiment comprises cyclosporin A, Glucopon 225 DK®, and Mineral oil.

Another embodiment comprises cyclosporin A, Glucopon 425 N®, and Mineral oil.

Another embodiment comprises cyclosporin A, Glucopon 600 UP®, and Mineral oil.

Another embodiment comprises cyclosporin A, Glucopon 625 UP®, and Mineral oil.

Another embodiment comprises cyclosporin A, Pemulen 1621®, and Mineral oil.

Another embodiment comprises cyclosporin A, Pemulen 1622®, and Mineral oil.

Another embodiment comprises cyclosporin A, Pemulen TR-1®, and Mineral oil.

Another embodiment comprises cyclosporin A, Pemulen TR-2®, and Mineral oil.

Another embodiment comprises cyclosporin A, Plantacare 818®, and Mineral oil.

Another embodiment comprises cyclosporin A, Plantapon LGC Sorb®, and Mineral oil.

Another embodiment comprises cyclosporin A, Plantaren 1200N®, and Mineral oil.

Another embodiment comprises cyclosporin A, Plantaren 2000N®, and Mineral oil.

Another embodiment comprises cyclosporin A, Viscolam AT 64®, and Mineral oil.

Another embodiment comprises cyclosporin A, Viscolam AT 64P®, and Mineral oil.

Another embodiment comprises cyclosporin A, Viscolam AT 100®, and Mineral oil.

Another embodiment comprises cyclosporin A, Viscolam MAC 7®, and Mineral oil.

Another embodiment comprises cyclosporin A, Viscolam SMC 20®, and Mineral oil.

Another embodiment comprises cyclosporin A, Witbreak RTC-323®, and Mineral oil.

Another embodiment comprises cyclosporin A, WSI 3700®, and Mineral oil.

Another embodiment comprises cyclosporin A, a Propoxylated or Ethoxylated Fatty Acid, Alcohol, or Alkyl Phenol, and Mineral oil.

Another embodiment comprises cyclosporin A, Antarox AA-60®, and Mineral oil.

Another embodiment comprises cyclosporin A, Antarox LF-224®, and Mineral oil.

Another embodiment comprises cyclosporin A, Burcomul DFE-45®, and Mineral oil.

Another embodiment comprises cyclosporin A, Burcoterge LFE-1000®, and Mineral oil.

Another embodiment comprises cyclosporin A, Chemal LF-25B®, and Mineral oil.

Another embodiment comprises cyclosporin A, Chemal LF-40B®, and Mineral oil.

Another embodiment comprises cyclosporin A, Dehypon LS-36®, and Mineral oil.

Another embodiment comprises cyclosporin A, Dehypon LS-36®, and Mineral oil.

Another embodiment comprises cyclosporin A, Dehypon LS-54®, and Mineral oil.

Another embodiment comprises cyclosporin A, Dehypon LS-54®, and Mineral oil.

Another embodiment comprises cyclosporin A, Delonic 100 VLF®, and Mineral oil.

Another embodiment comprises cyclosporin A, Delonic LF-60 MOD®, and Mineral oil.

Another embodiment comprises cyclosporin A, Empiderm B®, and Mineral oil.

Another embodiment comprises cyclosporin A, Ethylan 1206®, and Mineral oil.

Another embodiment comprises cyclosporin A, Ethylan NS-500K®, and Mineral oil.

Another embodiment comprises cyclosporin A, Ethylan NS-500LQ®, and Mineral oil.

Another embodiment comprises cyclosporin A, Genapol 1392®, and Mineral oil.

Another embodiment comprises cyclosporin A, Genapol 2317®, and Mineral oil.

Another embodiment comprises cyclosporin A, Genapol 26EP710®, and Mineral oil.

Another embodiment comprises cyclosporin A, Genapol EP 1022®, and Mineral oil.

Another embodiment comprises cyclosporin A, Genapol EP 1024®, and Mineral oil.

Another embodiment comprises cyclosporin A, Genapol EP 6068®, and Mineral oil.

Another embodiment comprises cyclosporin A, Genapol NP915®, and Mineral oil.

Another embodiment comprises cyclosporin A, Kieralon MFB®, and Mineral oil.

Another embodiment comprises cyclosporin A, Lumisolve CSA-80 V®, and Mineral oil.

Another embodiment comprises cyclosporin A, Marlowet 5001®, and Mineral oil.

Another embodiment comprises cyclosporin A, Marlox FK 64®, and Mineral oil.

Another embodiment comprises cyclosporin A, Marlox MO 124®, and Mineral oil.

Another embodiment comprises cyclosporin A, Marlox S 58®, and Mineral oil.

Another embodiment comprises cyclosporin A, Nonatell 1003®, and Mineral oil.

Another embodiment comprises cyclosporin A, Nonatell 1038®, and Mineral oil.

Another embodiment comprises cyclosporin A, Nonatell 1052®, and Mineral oil.

Another embodiment comprises cyclosporin A, Nonatell 1061®, and Mineral oil.

Another embodiment comprises cyclosporin A, Nonatell 1075®, and Mineral oil.

Another embodiment comprises cyclosporin A, Nonatell 1088®, and Mineral oil.

Another embodiment comprises cyclosporin A, Nonatell 1123®, and Mineral oil.

Another embodiment comprises cyclosporin A, Nonatell 1153®, and Mineral oil.

Another embodiment comprises cyclosporin A, Nonatell 1161®, and Mineral oil.

Another embodiment comprises cyclosporin A, Nonatell 1172®, and Mineral oil.

Another embodiment comprises cyclosporin A, Nonatell 1181®, and Mineral oil.

Another embodiment comprises cyclosporin A, Norfox 36®, and Mineral oil.

Another embodiment comprises cyclosporin A, Procetyl AWS®, and Mineral oil.

Another embodiment comprises cyclosporin A, Sandoxylate SX 412®, and Mineral oil.

Another embodiment comprises cyclosporin A, Sandoxylate SX 418®, and Mineral oil.

Another embodiment comprises cyclosporin A, Surfonic JL-80X®, and Mineral oil.

Another embodiment comprises cyclosporin A, Surfonic JL-80X-B1®, and Mineral oil.

Another embodiment comprises cyclosporin A, Surfonic L4-29X®, and Mineral oil.

Another embodiment comprises cyclosporin A, Surfonic LF®, and Mineral oil.

Another embodiment comprises cyclosporin A, T-Det A826®, and Mineral oil.

Another embodiment comprises cyclosporin A, T-Det LF-416®, and Mineral oil.

Another embodiment comprises cyclosporin A, Tergitol Min-Foam 1 X®, and Mineral oil.

Another embodiment comprises cyclosporin A, Tergitol Min-Foam 2X®, and Mineral oil.

Another embodiment comprises cyclosporin A, Triton CF-21®, and Mineral oil.

Another embodiment comprises cyclosporin A, Triton CF-76®, and Mineral oil.

Another embodiment comprises cyclosporin A, Triton XL-80N®, and Mineral oil.

Another embodiment comprises cyclosporin A, Witconol NS-98®, and Mineral oil.

Another embodiment comprises cyclosporin A, Witconol NS-108LQ®, and Mineral oil.

Another embodiment comprises cyclosporin A, Witconol NS-145®, and Mineral oil.

Another embodiment comprises cyclosporin A, Witconol NS-179®, and Mineral oil.

Another embodiment comprises cyclosporin A, a Protein-based Surfactant, and Mineral oil.

Another embodiment comprises cyclosporin A, AminoFoam W®, and Mineral oil.

Another embodiment comprises cyclosporin A, Amiter LGOD-2®, and Mineral oil.

Another embodiment comprises cyclosporin A, Amiter LGS-2®, and Mineral oil.

Another embodiment comprises cyclosporin A, Amiter LGS-5®, and Mineral oil.

Another embodiment comprises cyclosporin A, Lamepon S®, and Mineral oil.

Another embodiment comprises cyclosporin A, Lamepon S®, and Mineral oil.

Another embodiment comprises cyclosporin A, Maypon 4C®, and Mineral oil.

Another embodiment comprises cyclosporin A, May-Tein C®, and Mineral oil.

Another embodiment comprises cyclosporin A, May-Tein CT®, and Mineral oil.

Another embodiment comprises cyclosporin A, May-Tein KTS®, and Mineral oil.

Another embodiment comprises cyclosporin A, May-Tein SY®, and Mineral oil.

Another embodiment comprises cyclosporin A, Plantapon S®, and Mineral oil.

Another embodiment comprises cyclosporin A, Proteol APL®, and Mineral oil.

Another embodiment comprises cyclosporin A, Proteol OAT®, and Mineral oil.

Another embodiment comprises cyclosporin A, Pyroter CPI-40®, and Mineral oil.

Another embodiment comprises cyclosporin A, Pyroter GPI-25®, and Mineral oil.

Another embodiment comprises cyclosporin A, Supro-Tein S®, and Mineral oil.

Another embodiment comprises cyclosporin A, Supro-Tein V®, and Mineral oil.

Another embodiment comprises cyclosporin A,® and Mineral oil.

Another embodiment comprises cyclosporin A, a Sarcosine Derivative®, and Mineral oil.

Another embodiment comprises cyclosporin A, Crodasinic LS-30®, and Mineral oil.

Another embodiment comprises cyclosporin A, Vanseal CS®, and Mineral oil.

Another embodiment comprises cyclosporin A, Vanseal LS®, and Mineral oil.

Another embodiment comprises cyclosporin A, Vanseal MS®, and Mineral oil.

Another embodiment comprises cyclosporin A, Vanseal NACS-30®, and Mineral oil.

Another embodiment comprises cyclosporin A, Vanseal NALS-95®, and Mineral oil.

Another embodiment comprises cyclosporin A, Vanseal OS®, and Mineral oil.

Another embodiment comprises cyclosporin A,® and Mineral oil.

Another embodiment comprises cyclosporin A, a Silicone-based Surfactant®, and Mineral oil.

Another embodiment comprises cyclosporin A, Abil-B-9950®, and Mineral oil.

Another embodiment comprises cyclosporin A, Abil Care 85®, and Mineral oil.

Another embodiment comprises cyclosporin A, Abil EM 90®, and Mineral oil.

Another embodiment comprises cyclosporin A, Abil EM 97®, and Mineral oil.

Another embodiment comprises cyclosporin A, Abil WE-09®, and Mineral oil.

Another embodiment comprises cyclosporin A, Dow Corning 1248 Fluid®, and Mineral oil.

Another embodiment comprises cyclosporin A, Dow Corning 3225C®, and Mineral oil.

Another embodiment comprises cyclosporin A, Dow Corning 5200®, and Mineral oil.

Another embodiment comprises cyclosporin A, Dow Corning Q4-3667®, and Mineral oil.

Another embodiment comprises cyclosporin A, Monasil PCA®, and Mineral oil.

Another embodiment comprises cyclosporin A, Monasil PDM®, and Mineral oil.

Another embodiment comprises cyclosporin A, Monasil PLN®, and Mineral oil.

Another embodiment comprises cyclosporin A, Polyderm PPI-SI-WS®, and Mineral oil.

Another embodiment comprises cyclosporin A, Troysol 380W®, and Mineral oil.

Another embodiment comprises cyclosporin A, Troysol S366®, and Mineral oil.

Another embodiment comprises cyclosporin A, a Sorbitan Derivative, and Mineral oil.

Another embodiment comprises cyclosporin A, Alkamuls SML®, and Mineral oil.

Another embodiment comprises cyclosporin A, Alkamuls SMO®, and Mineral oil.

Another embodiment comprises cyclosporin A, Alkamuls STO®, and Mineral oil.

Another embodiment comprises cyclosporin A, Arlacel 20®, and Mineral oil.

Another embodiment comprises cyclosporin A, Arlacel 40®, and Mineral oil.

Another embodiment comprises cyclosporin A, Arlacel 60®, and Mineral oil.

Another embodiment comprises cyclosporin A, Arlacel 80®, and Mineral oil.

Another embodiment comprises cyclosporin A, Arlacel C®, and Mineral oil.

Another embodiment comprises cyclosporin A, Armul 21®, and Mineral oil.

Another embodiment comprises cyclosporin A, Atlox 80®, and Mineral oil.

Another embodiment comprises cyclosporin A, Atlox 847®, and Mineral oil.

Another embodiment comprises cyclosporin A, Atlox 1045A®, and Mineral oil.

Another embodiment comprises cyclosporin A, Canarcel 20®, and Mineral oil.

Another embodiment comprises cyclosporin A, Canarcel 60®, and Mineral oil.

Another embodiment comprises cyclosporin A, Canarcel 80®, and Mineral oil.

Another embodiment comprises cyclosporin A, Canarcel TW 20®, and Mineral oil.

Another embodiment comprises cyclosporin A, Canarcel TW 60®, and Mineral oil.

Another embodiment comprises cyclosporin A, Canarcel TW 80®, and Mineral oil.

Another embodiment comprises cyclosporin A, Coladet BSB-P®, and Mineral oil.

Another embodiment comprises cyclosporin A, Customulse O-20®, and Mineral oil.

Another embodiment comprises cyclosporin A, Dehymuls E®, and Mineral oil.

Another embodiment comprises cyclosporin A, DeSotan SMO®, and Mineral oil.

Another embodiment comprises cyclosporin A, DeSotan SMO-20®, and Mineral oil.

Another embodiment comprises cyclosporin A, DeSotan SMT®, and Mineral oil.

Another embodiment comprises cyclosporin A, DeSotan SMT-20®, and Mineral oil.

Another embodiment comprises cyclosporin A, Durfax 60®, and Mineral oil.

Another embodiment comprises cyclosporin A, Durfax 65®, and Mineral oil.

Another embodiment comprises cyclosporin A, Durfax 80®, and Mineral oil.

Another embodiment comprises cyclosporin A, Durtan 60®, and Mineral oil.

Another embodiment comprises cyclosporin A, Durtan 65®, and Mineral oil.

Another embodiment comprises cyclosporin A, Liposorb L®, and Mineral oil.

Another embodiment comprises cyclosporin A, Liposorb L-10®, and Mineral oil.

Another embodiment comprises cyclosporin A, Liposorb L-20®, and Mineral oil.

Another embodiment comprises cyclosporin A, Liposorb O®, and Mineral oil.

Another embodiment comprises cyclosporin A, Liposorb O-20®, and Mineral oil.

Another embodiment comprises cyclosporin A, Liposorb P®, and Mineral oil.

Another embodiment comprises cyclosporin A, Liposorb P-20®, and Mineral oil.

Another embodiment comprises cyclosporin A, Liposorb S®, and Mineral oil.

Another embodiment comprises cyclosporin A, Liposorb S-20®, and Mineral oil.

Another embodiment comprises cyclosporin A, Liposorb SQO®, and Mineral oil.

Another embodiment comprises cyclosporin A, Liposorb TO®, and Mineral oil.

Another embodiment comprises cyclosporin A, Liposorb TS®, and Mineral oil.

Another embodiment comprises cyclosporin A, Liposorb TS-20®, and Mineral oil.

Another embodiment comprises cyclosporin A, Lumisorb PS®, and Mineral oil.

Another embodiment comprises cyclosporin A, Lumisorb SMO (T)®, and Mineral oil.

Another embodiment comprises cyclosporin A, Lumisorb SMS K®, and Mineral oil.

Another embodiment comprises cyclosporin A, Lumisorb SSO®, and Mineral oil.

Another embodiment comprises cyclosporin A, Lumisorb STS K®, and Mineral oil.

Another embodiment comprises cyclosporin A, Lumisorb STT®, and Mineral oil.

Another embodiment comprises cyclosporin A, Magrabar SMO®, and Mineral oil.

Another embodiment comprises cyclosporin A, Magrabar SMO-VEG®, and Mineral oil.

Another embodiment comprises cyclosporin A, Magrabar SMT®, and Mineral oil.

Another embodiment comprises cyclosporin A, Magrabar STO®, and Mineral oil.

Another embodiment comprises cyclosporin A, Miracare BC-27®, and Mineral oil.

Another embodiment comprises cyclosporin A, Ritabate 20®, and Mineral oil.

Another embodiment comprises cyclosporin A, Ritabate 40®, and Mineral oil.

Another embodiment comprises cyclosporin A, Ritabate 60®, and Mineral oil.

Another embodiment comprises cyclosporin A, Ritabate 80®, and Mineral oil.

Another embodiment comprises cyclosporin A, T-Maz®, and Mineral oil.

Another embodiment comprises cyclosporin A, Tego SML®, and Mineral oil.

Another embodiment comprises cyclosporin A, Tego SML 20®, and Mineral oil.

Another embodiment comprises cyclosporin A, Tego SMO 80 V®, and Mineral oil.

Another embodiment comprises cyclosporin A, Tego SMO V®, and Mineral oil.

Another embodiment comprises cyclosporin A, Tego SMS®, and Mineral oil.

Another embodiment comprises cyclosporin A, Tego STO V®, and Mineral oil.

Another embodiment comprises cyclosporin A, Tween 21®, and Mineral oil.

Another embodiment comprises cyclosporin A, Tween 40®, and Mineral oil.

Another embodiment comprises cyclosporin A, Tween 60®, and Mineral oil.

Another embodiment comprises cyclosporin A, Tween 60 K®, and Mineral oil.

Another embodiment comprises cyclosporin A, Tween 61®, and Mineral oil.

Another embodiment comprises cyclosporin A, Tween 65®, and Mineral oil.

Another embodiment comprises cyclosporin A, Tween 80®, and Mineral oil.

Another embodiment comprises cyclosporin A, Tween 80 K®, and Mineral oil.

Another embodiment comprises cyclosporin A, Tween 81®, and Mineral oil.

Another embodiment comprises cyclosporin A, Tween 85®, and Mineral oil.

Another embodiment comprises cyclosporin A, a Sucrose or Glucose Ester, or Derivative thereof, and Mineral oil.

Another embodiment comprises cyclosporin A, DeSulf GOS-P-60WCG®, and Mineral oil.

Another embodiment comprises cyclosporin A, Glucam E-20 Distearate®, and Mineral oil.

Another embodiment comprises cyclosporin A, Glucamate DOE-120®, and Mineral oil.

Another embodiment comprises cyclosporin A, Glucamate SSE-20®, and Mineral oil.

Another embodiment comprises cyclosporin A, Glucate DO®, and Mineral oil.

Another embodiment comprises cyclosporin A, Glucate SS®, and Mineral oil.

Another embodiment comprises cyclosporin A, Glucopon 425 UP®, and Mineral oil.

Another embodiment comprises cyclosporin A, Isolan IS®, and Mineral oil.

Another embodiment comprises cyclosporin A, Mazon 40®, and Mineral oil.

Another embodiment comprises cyclosporin A, Montanov 82®, and Mineral oil.

Another embodiment comprises cyclosporin A, Montanov 202®, and Mineral oil.

Another embodiment comprises cyclosporin A, Montanov S®, and Mineral oil.

Another embodiment comprises cyclosporin A, Rheozan®, and Mineral oil.

Another embodiment comprises cyclosporin A, Simulsol AS 48®, and Mineral oil.

Another embodiment comprises cyclosporin A, Simulsol SL 4®, and Mineral oil.

Another embodiment comprises cyclosporin A, Simulsol SL 10®, and Mineral oil.

Another embodiment comprises cyclosporin A, Simulsol SL 11W®, and Mineral oil.

Another embodiment comprises cyclosporin A, Simulsol SL 55®, and Mineral oil.

Another embodiment comprises cyclosporin A, Suga Nate 100 and 160®, and Mineral oil.

Another embodiment comprises cyclosporin A, Tego Care 450®, and Mineral oil.

Another embodiment comprises cyclosporin A, Tego Care CG 90®, and Mineral oil.

Another embodiment comprises cyclosporin A, Tego Care PS®, and Mineral oil.

Another embodiment comprises cyclosporin A, Tegosoft PSE 141 G®, and Mineral oil.

Another embodiment comprises cyclosporin A, Tegotens G 826®, and Mineral oil.

Another embodiment comprises cyclosporin A, Triton BG-10 (70%)®, and Mineral oil.

Another embodiment comprises cyclosporin A, Triton CG-110 (60%)®, and Mineral oil.

Another embodiment comprises cyclosporin A, Wickenol 545®, and Mineral oil.

Another embodiment comprises cyclosporin A, an alcohol, and Coriander oil.

Another embodiment comprises cyclosporin A, Diglycerol®, and Coriander oil.

Another embodiment comprises cyclosporin A, Hetoxide GT-80®, and Coriander oil.

Another embodiment comprises cyclosporin A, Lexemul BEO®, and Coriander oil.

Another embodiment comprises cyclosporin A, Polyglycerol-3®, and Coriander oil.

Another embodiment comprises cyclosporin A, Redicote E Series®, and Coriander oil.

Another embodiment comprises cyclosporin A, Simulsol OX 1005L®, and Coriander oil.

Another embodiment comprises cyclosporin A, Stanfax 567®, and Coriander oil.

Another embodiment comprises cyclosporin A, TA-1618®, and Coriander oil.

Another embodiment comprises cyclosporin A, Witconol H-31A®, and Coriander oil.

Another embodiment comprises cyclosporin A, an amine oxide, and Coriander oil.

Another embodiment comprises cyclosporin A, AO-405®, and Coriander oil.

Another embodiment comprises cyclosporin A, AO-455®, and Coriander oil.

Another embodiment comprises cyclosporin A, AO 728 Specials, and Coriander oil.

Another embodiment comprises cyclosporin A, Barlox 12®, and Coriander oil.

Another embodiment comprises cyclosporin A, Barlox 14®, and Coriander oil.

Another embodiment comprises cyclosporin A, Burcoxide Lo®, and Coriander oil.

Another embodiment comprises cyclosporin A, Caloxamine LO®, and Coriander oil.

Another embodiment comprises cyclosporin A, Chemoxide CAW®, and Coriander oil.

Another embodiment comprises cyclosporin A, Chemoxide LM-30®, and Coriander oil.

Another embodiment comprises cyclosporin A, Chemoxide LO®, and Coriander oil.

Another embodiment comprises cyclosporin A, Chemoxide MO®, and Coriander oil.

Another embodiment comprises cyclosporin A, Colalux CAO-35®, and Coriander oil.

Another embodiment comprises cyclosporin A, Colalux LO®, and Coriander oil.

Another embodiment comprises cyclosporin A, DeMox CAPO®, and Coriander oil.

Another embodiment comprises cyclosporin A, DeMox CSG-30®, and Coriander oil.

Another embodiment comprises cyclosporin A, DeMox LAO®, and Coriander oil.

Another embodiment comprises cyclosporin A, Emcol L®, and Coriander oil.

Another embodiment comprises cyclosporin A, Empigen OB®, and Coriander oil.

Another embodiment comprises cyclosporin A, Empigen OS/A®, and Coriander oil.

Another embodiment comprises cyclosporin A, Foamox CDO®, and Coriander oil.

Another embodiment comprises cyclosporin A, Foamox DMM®, and Coriander oil.

Another embodiment comprises cyclosporin A, Foamox DMS®, and Coriander oil.

Another embodiment comprises cyclosporin A, Genaminox KC®, and Coriander oil.

Another embodiment comprises cyclosporin A, Genaminox LA®, and Coriander oil.

Another embodiment comprises cyclosporin A, Hartotoam SAO®, and Coriander oil.

Another embodiment comprises cyclosporin A, Hartox DMCD®, and Coriander oil.

Another embodiment comprises cyclosporin A, Lipowax DAT®, and Coriander oil.

Another embodiment comprises cyclosporin A, Lipowax PB Pastilles®, and Coriander oil.

Another embodiment comprises cyclosporin A, Mackamine C8®, and Coriander oil.

Another embodiment comprises cyclosporin A, Mackamine C10®, and Coriander oil.

Another embodiment comprises cyclosporin A, Mackamine C14®, and Coriander oil.

Another embodiment comprises cyclosporin A, Mackamine CAO®, and Coriander oil.

Another embodiment comprises cyclosporin A, Mackamine CO®, and Coriander oil.

Another embodiment comprises cyclosporin A, Mackamine LO®, and Coriander oil.

Another embodiment comprises cyclosporin A, Mackamine O2®, and Coriander oil.

Another embodiment comprises cyclosporin A, Mackamine SAO®, and Coriander oil.

Another embodiment comprises cyclosporin A, Mackamine SO®, and Coriander oil.

Another embodiment comprises cyclosporin A, Mazox KCAO®, and Coriander oil.

Another embodiment comprises cyclosporin A, Monalac MO®, and Coriander oil.

Another embodiment comprises cyclosporin A, Nonfox LDA®, and Coriander oil.

Another embodiment comprises cyclosporin A, Rhodamox LO®, and Coriander oil.

Another embodiment comprises cyclosporin A, Schercamox C-AA®, and Coriander oil.

Another embodiment comprises cyclosporin A, Schercamox DMA®, and Coriander oil.

Another embodiment comprises cyclosporin A, Schercamox DML®, and Coriander oil.

Another embodiment comprises cyclosporin A, Schercamox DMM®, and Coriander oil.

Another embodiment comprises cyclosporin A, Schercamox DMS®, and Coriander oil.

Another embodiment comprises cyclosporin A, Tegotens DO®, and Coriander oil.

Another embodiment comprises cyclosporin A, Tomah AO-14-2®, and Coriander oil.

Another embodiment comprises cyclosporin A, Triaminox CDO®, and Coriander oil.

Another embodiment comprises cyclosporin A, a block polymer, and Coriander oil.

Another embodiment comprises cyclosporin A, AL 2070®, and Coriander oil.

Another embodiment comprises cyclosporin A, Antarox 17-R-2®, and Coriander oil.

Another embodiment comprises cyclosporin A, Antarox 25-R-2®, and Coriander oil.

Another embodiment comprises cyclosporin A, Antarox 31-R-1®, and Coriander oil.

Another embodiment comprises cyclosporin A, Antarox P-84®, and Coriander oil.

Another embodiment comprises cyclosporin A, Antarox P-104/H®, and Coriander oil.

Another embodiment comprises cyclosporin A, Arnox BP-Series®, and Coriander oil.

Another embodiment comprises cyclosporin A, Chemonic 435®, and Coriander oil.

Another embodiment comprises cyclosporin A, Chemonic D-25®, and Coriander oil.

Another embodiment comprises cyclosporin A, Chemonic PL Series®, and Coriander oil.

Another embodiment comprises cyclosporin A, Ethox L-121®, and Coriander oil.

Another embodiment comprises cyclosporin A, Ethox L-122®, and Coriander oil.

Another embodiment comprises cyclosporin A, Genapol PF-10®, and Coriander oil.

Another embodiment comprises cyclosporin A, Genapol PF-20®, and Coriander oil.

Another embodiment comprises cyclosporin A, Genapol PF-40A®, and Coriander oil.

Another embodiment comprises cyclosporin A, Norfox 2-LF®, and Coriander oil.

Another embodiment comprises cyclosporin A, Simulsol NW 342®, and Coriander oil.

Another embodiment comprises cyclosporin A, T-Det BP-1®, and Coriander oil.

Another embodiment comprises cyclosporin A, T-Det XD®, and Coriander oil.

Another embodiment comprises cyclosporin A, T-Det XH®, and Coriander oil.

Another embodiment comprises cyclosporin A, Triton CF-32®, and Coriander oil.

Another embodiment comprises cyclosporin A, Witconol 171®, and Coriander oil.

Another embodiment comprises cyclosporin A, Witconol 324®, and Coriander oil.

Another embodiment comprises cyclosporin A, Witconol 324D®, and Coriander oil.

Another embodiment comprises cyclosporin A, Witconol PD-2000®, and Coriander oil.

Another embodiment comprises cyclosporin A, a Carboxylated Alcohol or Alkylphenol Ethoxylate, and Coriander oil.

Another embodiment comprises cyclosporin A, Emcol CN-6®, and Coriander oil.

Another embodiment comprises cyclosporin A, Ethcarb®, and Coriander oil.

Another embodiment comprises cyclosporin A, Gemtex WNT-Conc®, and Coriander oil.

Another embodiment comprises cyclosporin A, Incrodet TD7-C®, and Coriander oil.

Another embodiment comprises cyclosporin A, Marlinat CM 105/80®, and Coriander oil.

Another embodiment comprises cyclosporin A, Marlowet 1072®, and Coriander oil.

Another embodiment comprises cyclosporin A, Marlowet 4530®, and Coriander oil.

Another embodiment comprises cyclosporin A, Marlowet 4530 LF®, and Coriander oil.

Another embodiment comprises cyclosporin A, Marlowet 4534®, and Coriander oil.

Another embodiment comprises cyclosporin A, Marlowet 4538®, and Coriander oil.

Another embodiment comprises cyclosporin A, Marlowet 4539®, and Coriander oil.

Another embodiment comprises cyclosporin A, Marlowet 4539 LF®, and Coriander oil.

Another embodiment comprises cyclosporin A, Marlowet 4541®, and Coriander oil.

Another embodiment comprises cyclosporin A, Miranate LEC-80®, and Coriander oil.

Another embodiment comprises cyclosporin A, Sandopan B®, and Coriander oil.

Another embodiment comprises cyclosporin A, Sandopan B Modified®, and Coriander oil.

Another embodiment comprises cyclosporin A, Sandopan LS-24 Gel®, and Coriander oil.

Another embodiment comprises cyclosporin A, Surfine T-A®, and Coriander oil.

Another embodiment comprises cyclosporin A, a Carboxylic Acid/Fatty Acid, and Coriander oil.

Another embodiment comprises cyclosporin A, Colaterge RAM®, and Coriander oil.

Another embodiment comprises cyclosporin A, Colatrope INC®, and Coriander oil.

Another embodiment comprises cyclosporin A, Crodacid B®, and Coriander oil.

Another embodiment comprises cyclosporin A, DeTrope CA-100®, and Coriander oil.

Another embodiment comprises cyclosporin A, Latol MTO®, and Coriander oil.

Another embodiment comprises cyclosporin A, Lumulse CC-33 K®, and Coriander oil.

Another embodiment comprises cyclosporin A, Mulls 2218®, and Coriander oil.

Another embodiment comprises cyclosporin A, OL-600®, and Coriander oil.

Another embodiment comprises cyclosporin A, OL-800®, and Coriander oil.

Another embodiment comprises cyclosporin A, R-910®, and Coriander oil.

Another embodiment comprises cyclosporin A, S-210®, and Coriander oil.

Another embodiment comprises cyclosporin A, Sandopan DTC Acid®, and Coriander oil.

Another embodiment comprises cyclosporin A, Sandopan LS 24 N®, and Coriander oil.

Another embodiment comprises cyclosporin A, Sandopan MA-18®, and Coriander oil.

Another embodiment comprises cyclosporin A, an Ethoxylated Alcohol, and Coriander oil.

Another embodiment comprises cyclosporin A, Adsee 799®, and Coriander oil.

Another embodiment comprises cyclosporin A, Adsee 799®, and Coriander oil.

Another embodiment comprises cyclosporin A, Alfonic 610-3.5®, and Coriander oil.

Another embodiment comprises cyclosporin A, Alfonic 810-2®, and Coriander oil.

Another embodiment comprises cyclosporin A, Alfonic 810-6®, and Coriander oil.

Another embodiment comprises cyclosporin A, Alfonic 1012-3®, and Coriander oil.

Another embodiment comprises cyclosporin A, Alfonic 1012-5®, and Coriander oil.

Another embodiment comprises cyclosporin A, Alfonic 1216CO-1.5®, and Coriander oil.

Another embodiment comprises cyclosporin A, Alfonic 1216CO-7®, and Coriander oil.

Another embodiment comprises cyclosporin A, Alfonic 1412-3®, and Coriander oil.

Another embodiment comprises cyclosporin A, Alfonic 1412-7®, and Coriander oil.

Another embodiment comprises cyclosporin A, Arlasolve 200®, and Coriander oil.

Another embodiment comprises cyclosporin A, Arlasolve 200 Liquid®, and Coriander oil.

Another embodiment comprises cyclosporin A, Armix 180-C®, and Coriander oil.

Another embodiment comprises cyclosporin A, Armix 183®, and Coriander oil.

Another embodiment comprises cyclosporin A, Armul 2404®, and Coriander oil.

Another embodiment comprises cyclosporin A, Armul 2404®, and Coriander oil.

Another embodiment comprises cyclosporin A, Atlas EMJ-C®, and Coriander oil.

Another embodiment comprises cyclosporin A, Atlas G-2109®, and Coriander oil.

Another embodiment comprises cyclosporin A, Atlas G-3886®, and Coriander oil.

Another embodiment comprises cyclosporin A, Atlas G-3890®, and Coriander oil.

Another embodiment comprises cyclosporin A, Bio Soft E-200®, and Coriander oil.

Another embodiment comprises cyclosporin A, Bio Soft E-300®, and Coriander oil.

Another embodiment comprises cyclosporin A, Bio Soft E-400®, and Coriander oil.

Another embodiment comprises cyclosporin A, Bio Soft EN 600®, and Coriander oil.

Another embodiment comprises cyclosporin A, Bio Soft TD-400®, and Coriander oil.

Another embodiment comprises cyclosporin A, Bio Soft TD-630®, and Coriander oil.

Another embodiment comprises cyclosporin A, Brij 30®, and Coriander oil.

Another embodiment comprises cyclosporin A, Brij 52®, and Coriander oil.

Another embodiment comprises cyclosporin A, Brij 56®, and Coriander oil.

Another embodiment comprises cyclosporin A, Brij 58®, and Coriander oil.

Another embodiment comprises cyclosporin A, Brij 72®, and Coriander oil.

Another embodiment comprises cyclosporin A, Brij 76®, and Coriander oil.

Another embodiment comprises cyclosporin A, Brij 78®, and Coriander oil.

Another embodiment comprises cyclosporin A, Brij 93®, and Coriander oil.

Another embodiment comprises cyclosporin A, Brij 97®, and Coriander oil.

Another embodiment comprises cyclosporin A, Brij 98®, and Coriander oil.

Another embodiment comprises cyclosporin A, Brij 700®, and Coriander oil.

Another embodiment comprises cyclosporin A, Brij 700 S®, and Coriander oil.

Another embodiment comprises cyclosporin A, Brij 721®, and Coriander oil.

Another embodiment comprises cyclosporin A, Brij 721 S®, and Coriander oil.

Another embodiment comprises cyclosporin A, Burcoterge CDG®, and Coriander oil.

Another embodiment comprises cyclosporin A, Canasol AT 600®, and Coriander oil.

Another embodiment comprises cyclosporin A, Canasol AT 800®, and Coriander oil.

Another embodiment comprises cyclosporin A, Canasol AT 1200®, and Coriander oil.

Another embodiment comprises cyclosporin A, Canasol BJ 35®, and Coriander oil.

Another embodiment comprises cyclosporin A, Canasol BJ 36®, and Coriander oil.

Another embodiment comprises cyclosporin A, Canasol BJ 52®, and Coriander oil.

Another embodiment comprises cyclosporin A, Canasol BJ 58®, and Coriander oil.

Another embodiment comprises cyclosporin A, Canasol BJ 72®, and Coriander oil.

Another embodiment comprises cyclosporin A, Canasol BJ 78®, and Coriander oil.

Another embodiment comprises cyclosporin A, Canasol BJ 98®, and Coriander oil.

Another embodiment comprises cyclosporin A, Canasol BJ 307®, and Coriander oil.

Another embodiment comprises cyclosporin A, Cerfak 1400®, and Coriander oil.

Another embodiment comprises cyclosporin A, Cetomacrogol 1000 BP®, and Coriander oil.

Another embodiment comprises cyclosporin A, Chemonic C-2®, and Coriander oil.

Another embodiment comprises cyclosporin A, Chemonic C-10®, and Coriander oil.

Another embodiment comprises cyclosporin A, Chemonic C-20®, and Coriander oil.

Another embodiment comprises cyclosporin A, Chemonic CT-12®, and Coriander oil.

Another embodiment comprises cyclosporin A, Chemonic CT-20®, and Coriander oil.

Another embodiment comprises cyclosporin A, Chemonic CT-30®, and Coriander oil.

Another embodiment comprises cyclosporin A, Chemonic CT-55®, and Coriander oil.

Another embodiment comprises cyclosporin A, Chemonic G-7®, and Coriander oil.

Another embodiment comprises cyclosporin A, Chemonic G-26®, and Coriander oil.

Another embodiment comprises cyclosporin A, Chemonic L-4®, and Coriander oil.

Another embodiment comprises cyclosporin A, Chemonic L-7®, and Coriander oil.

Another embodiment comprises cyclosporin A, Chemonic L-12®, and Coriander oil.

Another embodiment comprises cyclosporin A, Chemonic L-23®, and Coriander oil.

Another embodiment comprises cyclosporin A, Chemonic O-2®, and Coriander oil.

Another embodiment comprises cyclosporin A, Chemonic O-5®, and Coriander oil.

Another embodiment comprises cyclosporin A, Chemonic O-10®, and Coriander oil.

Another embodiment comprises cyclosporin A, Chemonic O-20®, and Coriander oil.

Another embodiment comprises cyclosporin A, Chemonic S-2®, and Coriander oil.

Another embodiment comprises cyclosporin A, Chemonic S-10®, and Coriander oil.

Another embodiment comprises cyclosporin A, Chemonic S-20®, and Coriander oil.

Another embodiment comprises cyclosporin A, Colamulse FE®, and Coriander oil.

Another embodiment comprises cyclosporin A, Cremophor A 20®, and Coriander oil.

Another embodiment comprises cyclosporin A, Cremophor SA 2®, and Coriander oil.

Another embodiment comprises cyclosporin A, Dehydol 100®, and Coriander oil.

Another embodiment comprises cyclosporin A, Dehydol O-4®, and Coriander oil.

Another embodiment comprises cyclosporin A, Delonic C-18®, and Coriander oil.

Another embodiment comprises cyclosporin A, DeSonic 6T®, and Coriander oil.

Another embodiment comprises cyclosporin A, DeSonic 9D®, and Coriander oil.

Another embodiment comprises cyclosporin A, DeSonic 9T®, and Coriander oil.

Another embodiment comprises cyclosporin A, DeSonic 12D®, and Coriander oil.

Another embodiment comprises cyclosporin A, DeSonic 12T®, and Coriander oil.

Another embodiment comprises cyclosporin A, DeSonic 15T®, and Coriander oil.

Another embodiment comprises cyclosporin A, DeSonic TDA-9®, and Coriander oil.

Another embodiment comprises cyclosporin A, DeThox GLG-7®, and Coriander oil.

Another embodiment comprises cyclosporin A, DeThox GLG-26®, and Coriander oil.

Another embodiment comprises cyclosporin A, DeThox LA-4®, and Coriander oil.

Another embodiment comprises cyclosporin A, DeThox LA-23®, and Coriander oil.

Another embodiment comprises cyclosporin A, DeThox SA-80®, and Coriander oil.

Another embodiment comprises cyclosporin A, Disponil O5®, and Coriander oil.

Another embodiment comprises cyclosporin A, Eccoterge EO-41B®, and Coriander oil.

Another embodiment comprises cyclosporin A, Empilan KA2.5/90®, and Coriander oil.

Another embodiment comprises cyclosporin A, Empilan KA5/90®, and Coriander oil.

Another embodiment comprises cyclosporin A, Empilan KM-20®, and Coriander oil.

Another embodiment comprises cyclosporin A, Empilan KM-50®, and Coriander oil.

Another embodiment comprises cyclosporin A, Empilan L-23®, and Coriander oil.

Another embodiment comprises cyclosporin A, Ethylan 25-3®, and Coriander oil.

Another embodiment comprises cyclosporin A, Ethylan 1204®, and Coriander oil.

Another embodiment comprises cyclosporin A, Ethylan DA-4®, and Coriander oil.

Another embodiment comprises cyclosporin A, Ethylan LA-230®, and Coriander oil.

Another embodiment comprises cyclosporin A, Ethylan SN®, and Coriander oil.

Another embodiment comprises cyclosporin A, Ethylan TD-60®, and Coriander oil.

Another embodiment comprises cyclosporin A, Ethylan TD-100®, and Coriander oil.

Another embodiment comprises cyclosporin A, Ethylan TD-1407®, and Coriander oil.

Another embodiment comprises cyclosporin A, Eumulgin B1®, and Coriander oil.

Another embodiment comprises cyclosporin A, Eumulgin B2®, and Coriander oil.

Another embodiment comprises cyclosporin A, Eumulgin B3®, and Coriander oil.

Another embodiment comprises cyclosporin A, Eumulgin O-10®, and Coriander oil.

Another embodiment comprises cyclosporin A, Flo Mo 80/20®, and Coriander oil.

Another embodiment comprises cyclosporin A, Flo Mo Low Foam®, and Coriander oil.

Another embodiment comprises cyclosporin A, Forlan C-24®, and Coriander oil.

Another embodiment comprises cyclosporin A, Genapol 1454®, and Coriander oil.

Another embodiment comprises cyclosporin A, Genapol BA-020®, and Coriander oil.

Another embodiment comprises cyclosporin A, Genapol BA-040®, and Coriander oil.

Another embodiment comprises cyclosporin A, Genapol C-100®, and Coriander oil.

Another embodiment comprises cyclosporin A, Genapol DA 060®, and Coriander oil.

Another embodiment comprises cyclosporin A, Genapol HS 020®, and Coriander oil.

Another embodiment comprises cyclosporin A, Genapol HS 200®, and Coriander oil.

Another embodiment comprises cyclosporin A, Genapol ID-040®, and Coriander oil.

Another embodiment comprises cyclosporin A, Genapol ID-060®, and Coriander oil.

Another embodiment comprises cyclosporin A, Genapol ID-090®, and Coriander oil.

Another embodiment comprises cyclosporin A, Genapol LA 010®, and Coriander oil.

Another embodiment comprises cyclosporin A, Genapol LA 020®, and Coriander oil.

Another embodiment comprises cyclosporin A, Genapol LA 030®, and Coriander oil.

Another embodiment comprises cyclosporin A, Genapol LA 040®, and Coriander oil.

Another embodiment comprises cyclosporin A, Genapol LA 050®, and Coriander oil.

Another embodiment comprises cyclosporin A, Genapol LA 060®, and Coriander oil.

Another embodiment comprises cyclosporin A, Genapol LA 070®, and Coriander oil.

Another embodiment comprises cyclosporin A, Genapol LA 070S®, and Coriander oil.

Another embodiment comprises cyclosporin A, Genapol LA 230®, and Coriander oil.

Another embodiment comprises cyclosporin A, Genapol O020®, and Coriander oil.

Another embodiment comprises cyclosporin A, Genapol O050®, and Coriander oil.

Another embodiment comprises cyclosporin A, Genapol O100®, and Coriander oil.

Another embodiment comprises cyclosporin A, Genapol O200®, and Coriander oil.

Another embodiment comprises cyclosporin A, Genapol SA 030®, and Coriander oil.

Another embodiment comprises cyclosporin A, Genapol SA 120®, and Coriander oil.

Another embodiment comprises cyclosporin A, Genapol T-020®, and Coriander oil.

Another embodiment comprises cyclosporin A, Genapol UD-030®, and Coriander oil.

Another embodiment comprises cyclosporin A, Genapol UD-050®, and Coriander oil.

Another embodiment comprises cyclosporin A, Genapol UD-070®, and Coriander oil.

Another embodiment comprises cyclosporin A, Genapol UD-079®, and Coriander oil.

Another embodiment comprises cyclosporin A, Genapol UD-080®, and Coriander oil.

Another embodiment comprises cyclosporin A, Genapol UD-110®, and Coriander oil.

Another embodiment comprises cyclosporin A, Genapol X 030®, and Coriander oil.

Another embodiment comprises cyclosporin A, Genapol X 050®, and Coriander oil.

Another embodiment comprises cyclosporin A, Genapol X 060®, and Coriander oil.

Another embodiment comprises cyclosporin A, Genapol X 070®, and Coriander oil.

Another embodiment comprises cyclosporin A, Genapol X 080®, and Coriander oil.

Another embodiment comprises cyclosporin A, Genapol X 100®, and Coriander oil.

Another embodiment comprises cyclosporin A, Genapol X 159®, and Coriander oil.

Another embodiment comprises cyclosporin A, Generol 122 E5®, and Coriander oil.

Another embodiment comprises cyclosporin A, Generol 122 E25®, and Coriander oil.

Another embodiment comprises cyclosporin A, Hostacerin T-3®, and Coriander oil.

Another embodiment comprises cyclosporin A, Iconol LF 110®, and Coriander oil.

Another embodiment comprises cyclosporin A, Incropol CS-20®, and Coriander oil.

Another embodiment comprises cyclosporin A, Lexemul CS-20®, and Coriander oil.

Another embodiment comprises cyclosporin A, Liponic EG-1®, and Coriander oil.

Another embodiment comprises cyclosporin A, Lipowax D®, and Coriander oil.

Another embodiment comprises cyclosporin A, Lipowax G®, and Coriander oil.

Another embodiment comprises cyclosporin A, Lipowax NI®, and Coriander oil.

Another embodiment comprises cyclosporin A, Lipowax P®, and Coriander oil.

Another embodiment comprises cyclosporin A, Lipowax P-31®, and Coriander oil.

Another embodiment comprises cyclosporin A, Lipowax PR®, and Coriander oil.

Another embodiment comprises cyclosporin A, Lumulse CS-20®, and Coriander oil.

Another embodiment comprises cyclosporin A, Macol CSA-20®, and Coriander oil.

Another embodiment comprises cyclosporin A, Marlox B 24/50®, and Coriander oil.

Another embodiment comprises cyclosporin A, Mazawet 77®, and Coriander oil.

Another embodiment comprises cyclosporin A, Norfox 1713®, and Coriander oil.

Another embodiment comprises cyclosporin A, Norfox 2579®, and Coriander oil.

Another embodiment comprises cyclosporin A, Norfox Lo Foam®, and Coriander oil.

Another embodiment comprises cyclosporin A, Promulgen D®, and Coriander oil.

Another embodiment comprises cyclosporin A, Promulgen G®, and Coriander oil.

Another embodiment comprises cyclosporin A, Renex 30®, and Coriander oil.

Another embodiment comprises cyclosporin A, Renex 36®, and Coriander oil.

Another embodiment comprises cyclosporin A, Rhodasurf A 24®, and Coriander oil.

Another embodiment comprises cyclosporin A, Rhodasurf AAE-10®, and Coriander oil.

Another embodiment comprises cyclosporin A, Rhodasurf BEH-25®, and Coriander oil.

Another embodiment comprises cyclosporin A, Rhodasurf BEH-40®, and Coriander oil.

Another embodiment comprises cyclosporin A, Rhodasurf DA 530®, and Coriander oil.

Another embodiment comprises cyclosporin A, Rhodasurf DA 630®, and Coriander oil.

Another embodiment comprises cyclosporin A, Rhodasurf DA 639®, and Coriander oil.

Another embodiment comprises cyclosporin A, Rhodasurf LAN-23®, and Coriander oil.

Another embodiment comprises cyclosporin A, Rhodasurf ON-870®, and Coriander oil.

Another embodiment comprises cyclosporin A, Rhodasurf ON-877®, and Coriander oil.

Another embodiment comprises cyclosporin A, Rhodasurf TB-970 FLK®, and Coriander oil.

Another embodiment comprises cyclosporin A, Ritacet-20®, and Coriander oil.

Another embodiment comprises cyclosporin A, Ritachol 1000®, and Coriander oil.

Another embodiment comprises cyclosporin A, Ritachol 2000®, and Coriander oil.

Another embodiment comprises cyclosporin A, Ritachol 5000®, and Coriander oil.

Another embodiment comprises cyclosporin A, Ritox 35®, and Coriander oil.

Another embodiment comprises cyclosporin A, Surfonic DA-4®, and Coriander oil.

Another embodiment comprises cyclosporin A, Surfonic DA-6®, and Coriander oil.

Another embodiment comprises cyclosporin A, Surfonic L46-7®, and Coriander oil.

Another embodiment comprises cyclosporin A, Surfonic POA®, and Coriander oil.

Another embodiment comprises cyclosporin A, Synthrapol KB®, and Coriander oil.

Another embodiment comprises cyclosporin A, Teginacid®, and Coriander oil.

Another embodiment comprises cyclosporin A, Teginacid C®, and Coriander oil.

Another embodiment comprises cyclosporin A, Tegotens EC 11®, and Coriander oil.

Another embodiment comprises cyclosporin A, Tinegal NA®, and Coriander oil.

Another embodiment comprises cyclosporin A, Tomadol 400®, and Coriander oil.

Another embodiment comprises cyclosporin A, Tomadol 600®, and Coriander oil.

Another embodiment comprises cyclosporin A, Tomadol 900®, and Coriander oil.

Another embodiment comprises cyclosporin A, Uniperol O®, and Coriander oil.

Another embodiment comprises cyclosporin A, Witconol SN Series®, and Coriander oil.

Another embodiment comprises cyclosporin A, an Ethoxylated Alkylphenol, and Coriander oil.

Another embodiment comprises cyclosporin A, Antarox LF-222®, and Coriander oil.

Another embodiment comprises cyclosporin A, Atlox 775®, and Coriander oil.

Another embodiment comprises cyclosporin A, Caloxylate N-9®, and Coriander oil.

Another embodiment comprises cyclosporin A, Canasol NF-1000®, and Coriander oil.

Another embodiment comprises cyclosporin A, Canasol NF-3000®, and Coriander oil.

Another embodiment comprises cyclosporin A, Canasol NF-3070®, and Coriander oil.

Another embodiment comprises cyclosporin A, Canasol OF 1670®, and Coriander oil.

Another embodiment comprises cyclosporin A, Canasol OF 2570®, and Coriander oil.

Another embodiment comprises cyclosporin A, Canasol OF 4070®, and Coriander oil.

Another embodiment comprises cyclosporin A, Chemax DNP-8®, and Coriander oil.

Another embodiment comprises cyclosporin A, Chemax DNP-18®, and Coriander oil.

Another embodiment comprises cyclosporin A, Chemax DNP-150/50®, and Coriander oil.

Another embodiment comprises cyclosporin A, DeSonic 1.5N®, and Coriander oil.

Another embodiment comprises cyclosporin A, DeSonic 4N®, and Coriander oil.

Another embodiment comprises cyclosporin A, DeSonic 5N®, and Coriander oil.

Another embodiment comprises cyclosporin A, DeSonic 6D®, and Coriander oil.

Another embodiment comprises cyclosporin A, DeSonic 6N®, and Coriander oil.

Another embodiment comprises cyclosporin A, DeSonic 7N®, and Coriander oil.

Another embodiment comprises cyclosporin A, DeSonic 9N®, and Coriander oil.

Another embodiment comprises cyclosporin A, DeSonic 10D®, and Coriander oil.

Another embodiment comprises cyclosporin A, DeSonic 11N®, and Coriander oil.

Another embodiment comprises cyclosporin A, DeSonic 12N®, and Coriander oil.

Another embodiment comprises cyclosporin A, DeSonic 15N®, and Coriander oil.

Another embodiment comprises cyclosporin A, DeSonic 20N®, and Coriander oil.

Another embodiment comprises cyclosporin A, Eccoscour RC®, and Coriander oil.

Another embodiment comprises cyclosporin A, Eccoterge EO-100®, and Coriander oil.

Another embodiment comprises cyclosporin A, Emulsifier 632/90%®, and Coriander oil.

Another embodiment comprises cyclosporin A, Geronol AG-821®, and Coriander oil.

Another embodiment comprises cyclosporin A, Gradonic N-95®, and Coriander oil.

Another embodiment comprises cyclosporin A, Hetoxide NP-4®, and Coriander oil.

Another embodiment comprises cyclosporin A, Hetoxide NP-30®, and Coriander oil.

Another embodiment comprises cyclosporin A, Hostapal N-100®, and Coriander oil.

Another embodiment comprises cyclosporin A, Hostapal N-110®, and Coriander oil.

Another embodiment comprises cyclosporin A, Igepal CTA-639W®, and Coriander oil.

Another embodiment comprises cyclosporin A, Igepal DAP-9®, and Coriander oil.

Another embodiment comprises cyclosporin A, Igepal OD-410®, and Coriander oil.

Another embodiment comprises cyclosporin A, Igepal SS-837®, and Coriander oil.

Another embodiment comprises cyclosporin A, Lipocol NP-9 USP®, and Coriander oil.

Another embodiment comprises cyclosporin A, Macol DNP-10®, and Coriander oil.

Another embodiment comprises cyclosporin A, Marlophen NP 5®, and Coriander oil.

Another embodiment comprises cyclosporin A, Marlophen P 1®, and Coriander oil.

Another embodiment comprises cyclosporin A, Surfonic NB®, and Coriander oil.

Another embodiment comprises cyclosporin A, Surfonic OPB-307®, and Coriander oil.

Another embodiment comprises cyclosporin A, Surfonic OPB-407®, and Coriander oil.

Another embodiment comprises cyclosporin A, Syn Fac 334®, and Coriander oil.

Another embodiment comprises cyclosporin A, Syn Fac 8216®, and Coriander oil.

Another embodiment comprises cyclosporin A, Triton N-57®, and Coriander oil.

Another embodiment comprises cyclosporin A, Trycol 6956®, and Coriander oil.

Another embodiment comprises cyclosporin A, Trycol 6961®, and Coriander oil.

Another embodiment comprises cyclosporin A, Trycol 6964®, and Coriander oil.

Another embodiment comprises cyclosporin A, Trycol 6969®, and Coriander oil.

Another embodiment comprises cyclosporin A, Trycol 6974®, and Coriander oil.

Another embodiment comprises cyclosporin A, Witbreak DRB-127®, and Coriander oil.

Another embodiment comprises cyclosporin A, Witbreak DRB-127®, and Coriander oil.

Another embodiment comprises cyclosporin A, Witconol NP Series®, and Coriander oil.

Another embodiment comprises cyclosporin A, an Ethoxylated Aryl Phenol, and Coriander oil.

Another embodiment comprises cyclosporin A, Soprophor BSU®, and Coriander oil.

Another embodiment comprises cyclosporin A, Soprophor CY/8®, and Coriander oil.

Another embodiment comprises cyclosporin A, Soprophor S/25®, and Coriander oil.

Another embodiment comprises cyclosporin A, Witconol NIO®, and Coriander oil.

Another embodiment comprises cyclosporin A, Witconol NIW®, and Coriander oil.

Another embodiment comprises cyclosporin A, Witconol S-100®, and Coriander oil.

Another embodiment comprises cyclosporin A, an Ethoxylated Fatty Acid, and Coriander oil.

Another embodiment comprises cyclosporin A, Aldo PGHMS®, and Coriander oil.

Another embodiment comprises cyclosporin A, Alkamuls TO-15/HR®, and Coriander oil.

Another embodiment comprises cyclosporin A, Armotan AL-69-66®, and Coriander oil.

Another embodiment comprises cyclosporin A, Cerasynt 840®, and Coriander oil.

Another embodiment comprises cyclosporin A, Cerasynt 945®, and Coriander oil.

Another embodiment comprises cyctosporin A, Crystal Inhibitor No. 5®, and Coriander oil.

Another embodiment comprises cyclosporin A, DeThox Acid L-9®, and Coriander oil.

Another embodiment comprises cyclosporin A, DeThox Acid S-8®, and Coriander oil.

Another embodiment comprises cyclosporin A, Ethofat 242/25®, and Coriander oil.

Another embodiment comprises cyclosporin A, Hydropalat 65®, and Coriander oil.

Another embodiment comprises cyclosporin A, Lipo EGMS®, and Coriander oil.

Another embodiment comprises cyclosporin A, Lipopeg 2 DL®, and Coriander oil.

Another embodiment comprises cyclosporin A, Lipopeg 4 DL®, and Coriander oil.

Another embodiment comprises cyclosporin A, Lipopeg 4-L®, and Coriander oil.

Another embodiment comprises cyclosporin A, Lipopeg 39-S®, and Coriander oil.

Another embodiment comprises cyclosporin A, Lipopeg 4-S®, and Coriander oil.

Another embodiment comprises cyclosporin A, Lipopeg 10-S®, and Coriander oil.

Another embodiment comprises cyclosporin A, Lipopeg 100-S®, and Coriander oil.

Another embodiment comprises cyclosporin A, Lipopeg 6000 DS®, and Coriander oil.

Another embodiment comprises cyclosporin A, Lumulse 40-L®, and Coriander oil.

Another embodiment comprises cyclosporin A, Lumulse 40-S®, and Coriander oil.

Another embodiment comprises cyclosporin A, Lumulse 42-L®, and Coriander oil.

Another embodiment comprises cyclosporin A, Lumulse 42-S®, and Coriander oil.

Another embodiment comprises cyclosporin A, Lumulse 100-S®, and Coriander oil.

Another embodiment comprises cyclosporin A, Lumulse 602-S®, and Coriander oil.

Another embodiment comprises cyclosporin A, Magrabar PGE-20-0®, and Coriander oil.

Another embodiment comprises cyclosporin A, Magrabar PGE-20L®, and Coriander oil.

Another embodiment comprises cyclosporin A, Magrabar PGE-20T®, and Coriander oil.

Another embodiment comprises cyclosporin A, Magrabar PGE-22-0®, and Coriander oil.

Another embodiment comprises cyclosporin A, Magrabar PGE-22L®, and Coriander oil.

Another embodiment comprises cyclosporin A, Magrabar PGE-22T®, and Coriander oil.

Another embodiment comprises cyclosporin A, Magrabar PGE-40-0®, and Coriander oil.

Another embodiment comprises cyclosporin A, Magrabar PGE-40L®, and Coriander oil.

Another embodiment comprises cyclosporin A, Magrabar PGE-40T®, and Coriander oil.

Another embodiment comprises cyclosporin A, Magrabar PGE-42-0®, and Coriander oil.

Another embodiment comprises cyclosporin A, Magrabar PGE-42L®, and Coriander oil.

Another embodiment comprises cyclosporin A, Magrabar PGE-42T®, and Coriander oil.

Another embodiment comprises cyclosporin A, Magrabar PGE-60-0®, and Coriander oil.

Another embodiment comprises cyclosporin A, Magrabar PGE-60L®, and Coriander oil.

Another embodiment comprises cyclosporin A, Magrabar PGE-60T®, and Coriander oil.

Another embodiment comprises cyclosporin A, Magrabar PGE-62-0®, and Coriander oil.

Another embodiment comprises cyclosporin A, Magrabar PGE-62L®, and Coriander oil.

Another embodiment comprises cyclosporin A, Magrabar PGE-62T®, and Coriander oil.

Another embodiment comprises cyclosporin A, Mapeg S-40K®, and Coriander oil.

Another embodiment comprises cyclosporin A, Marlowet OTS®, and Coriander oil.

Another embodiment comprises cyclosporin A, Naturechem PGR®, and Coriander oil.

Another embodiment comprises cyclosporin A, PG No. 4®, and Coriander oil.

Another embodiment comprises cyclosporin A, Renex 20®, and Coriander oil.

Another embodiment comprises cyclosporin A, Ritox 52®, and Coriander oil.

Another embodiment comprises cyclosporin A, Ritox 53®, and Coriander oil.

Another embodiment comprises cyclosporin A, Ritox 59®, and Coriander oil.

Another embodiment comprises cyclosporin A, Surfax 8916/A®, and Coriander oil.

Another embodiment comprises cyclosporin A, Tego Acid S 40 P®, and Coriander oil.

Another embodiment comprises cyclosporin A, Tego Acid S 100 P®, and Coriander oil.

Another embodiment comprises cyclosporin A, Tween 20®, and Coriander oil.

Another embodiment comprises cyclosporin A, Volpo 131®, and Coriander oil.

Another embodiment comprises cyclosporin A, an Ethoxylated Fatty Ester or Oil, and Coriander oil.

Another embodiment comprises cyclosporin A, Acconon 6-C10®, and Coriander oil.

Another embodiment comprises cyclosporin A, Acconon CC-6®, and Coriander oil.

Another embodiment comprises cyclosporin A, Acconon CO-7®, and Coriander oil.

Another embodiment comprises cyclosporin A, Aldosperse 40/60 FG®, and Coriander oil.

Another embodiment comprises cyclosporin A, Aldosperse ML-23®, and Coriander oil.

Another embodiment comprises cyclosporin A, Aldosperse MS-20 FG®, and Coriander oil.

Another embodiment comprises cyclosporin A, Alkamuls EL-620®, and Coriander oil.

Another embodiment comprises cyclosporin A, Alkamuls EL-719®, and Coriander oil.

Another embodiment comprises cyclosporin A, Alkamuls EL-985®, and Coriander oil.

Another embodiment comprises cyclosporin A, Arlatone G®, and Coriander oil.

Another embodiment comprises cyclosporin A, Arlatone T®, and Coriander oil.

Another embodiment comprises cyclosporin A, Atlas G-1045A®, and Coriander oil.

Another embodiment comprises cyclosporin A, Atlas G-1086®, and Coriander oil.

Another embodiment comprises cyclosporin A, Atlas G-1087®, and Coriander oil.

Another embodiment comprises cyclosporin A, Atlas G-1089®, and Coriander oil.

Another embodiment comprises cyclosporin A, Atlas G-1096®, and Coriander oil.

Another embodiment comprises cyclosporin A, Atlas G-1292®, and Coriander oil.

Another embodiment comprises cyclosporin A, Atlas G-1293®, and Coriander oil.

Another embodiment comprises cyclosporin A, Atlas G-1300®, and Coriander oil.

Another embodiment comprises cyclosporin A, Atlas G-7076®, and Coriander oil.

Another embodiment comprises cyclosporin A, Capmul EMG®, and Coriander oil.

Another embodiment comprises cyclosporin A, Chemonic CO-40®, and Coriander oil.

Another embodiment comprises cyclosporin A, Chemonic LI-3®, and Coriander oil.

Another embodiment comprises cyclosporin A, Chemonic LI-7®, and Coriander oil.

Another embodiment comprises cyclosporin A, Cirrasol GM®, and Coriander oil.

Another embodiment comprises cyclosporin A, Cremophor CO 40®, and Coriander oil.

Another embodiment comprises cyclosporin A, Cremophor CO 410®, and Coriander oil.

Another embodiment comprises cyclosporin A, Cremophor EL®, and Coriander oil.

Another embodiment comprises cyclosporin A, Cremophor GC7®, and Coriander oil.

Another embodiment comprises cyclosporin A, Cremophor RH-40®, and Coriander oil.

Another embodiment comprises cyclosporin A, Crovol A-40®, and Coriander oil.

Another embodiment comprises cyclosporin A, Crovol A-70®, and Coriander oil.

Another embodiment comprises cyclosporin A, Crovol M-70®, and Coriander oil.

Another embodiment comprises cyclosporin A, Crovol PK-70®, and Coriander oil.

Another embodiment comprises cyclosporin A, Cutina E-24®, and Coriander oil.

Another embodiment comprises cyclosporin A, Dacospin 12-R®, and Coriander oil.

Another embodiment comprises cyclosporin A, Dehymuls HRE-7®, and Coriander oil.

Another embodiment comprises cyclosporin A, DeSonic 30C®, and Coriander oil.

Another embodiment comprises cyclosporin A, DeSonic 36C®, and Coriander oil.

Another embodiment comprises cyclosporin A, DeSonic 40C®, and Coriander oil.

Another embodiment comprises cyclosporin A, Durfax 60®, and Coriander oil.

Another embodiment comprises cyclosporin A, Durfax 65®, and Coriander oil.

Another embodiment comprises cyclosporin A, Durfax 80®, and Coriander oil.

Another embodiment comprises cyclosporin A, Durfax EOM®, and Coriander oil.

Another embodiment comprises cyclosporin A, Eccoterge NF-2®, and Coriander oil.

Another embodiment comprises cyclosporin A, Emulpon CO-360®, and Coriander oil.

Another embodiment comprises cyclosporin A, Emulpon CO-550®, and Coriander oil.

Another embodiment comprises cyclosporin A, Emulsogen EL®, and Coriander oil.

Another embodiment comprises cyclosporin A, Emulsogen HCO 040®, and Coriander oil.

Another embodiment comprises cyclosporin A, Emulsogen HCO 060®, and Coriander oil.

Another embodiment comprises cyclosporin A, Emulsynt 1055®, and Coriander oil.

Another embodiment comprises cyclosporin A, Ethox 3095®, and Coriander oil.

Another embodiment comprises cyclosporin A, Eumulgin RO-40®, and Coriander oil.

Another embodiment comprises cyclosporin A, Genapol G-260®, and Coriander oil.

Another embodiment comprises cyclosporin A, Glycosperse L-20®, and Coriander oil.

Another embodiment comprises cyclosporin A, Glycosperse O-5®, and Coriander oil.

Another embodiment comprises cyclosporin A, Glycosperse O-20®, and Coriander oil.

Another embodiment comprises cyclosporin A, Glycosperse O-20 FG®, and Coriander oil.

Another embodiment comprises cyclosporin A, Glycosperse S-20®, and Coriander oil.

Another embodiment comprises cyclosporin A, Glycosperse S-20 FG®, and Coriander oil.

Another embodiment comprises cyclosporin A, Glycosperse TS-20®, and Coriander oil.

Another embodiment comprises cyclosporin A, Glycosperse TS-20 FG®, and Coriander oil.

Another embodiment comprises cyclosporin A, Hetan SL®, and Coriander oil.

Another embodiment comprises cyclosporin A, Hetan SO®, and Coriander oil.

Another embodiment comprises cyclosporin A, Hetan SS®, and Coriander oil.

Another embodiment comprises cyclosporin A, Hetoxide C-2®, and Coriander oil.

Another embodiment comprises cyclosporin A, Hetoxide C-9®, and Coriander oil.

Another embodiment comprises cyclosporin A, Hetoxide C-15®, and Coriander oil.

Another embodiment comprises cyclosporin A, Hetoxide C-25®, and Coriander oil.

Another embodiment comprises cyclosporin A, Hetoxide C-40®, and Coriander oil.

Another embodiment comprises cyclosporin A, Hetoxide C-200®, and Coriander oil.

Another embodiment comprises cyclosporin A, Hetoxide C-200-50%®, and Coriander oil.

Another embodiment comprises cyclosporin A, Hetoxide GC-30®, and Coriander oil.

Another embodiment comprises cyclosporin A, Hetoxide HC-60®, and Coriander oil.

Another embodiment comprises cyclosporin A, Ice No. 2®, and Coriander oil.

Another embodiment comprises cyclosporin A, Incrocas 30/40®, and Coriander oil.

Another embodiment comprises cyclosporin A, Lexol EC®, and Coriander oil.

Another embodiment comprises cyclosporin A, Lexol EO®, and Coriander oil.

Another embodiment comprises cyclosporin A, Lipocol HCO-40®, and Coriander oil.

Another embodiment comprises cyclosporin A, Lipocol HCO-60®, and Coriander oil.

Another embodiment comprises cyclosporin A, Lipocol O-3 Special®, and Coriander oil.

Another embodiment comprises cyclosporin A, Lipopeg 2-L®, and Coriander oil.

Another embodiment comprises cyclosporin A, Lipopeg 4-DO®, and Coriander oil.

Another embodiment comprises cyclosporin A, Lipopeg 4-DS®, and Coriander oil.

Another embodiment comprises cyclosporin A, Lipovol GTB®, and Coriander oil.

Another embodiment comprises cyclosporin A, Lonzest SML-20®, and Coriander oil.

Another embodiment comprises cyclosporin A, Lonzest SMO-20®, and Coriander oil.

Another embodiment comprises cyclosporin A, Lonzest SMS-20®, and Coriander oil.

Another embodiment comprises cyclosporin A, Lonzest STO-20®, and Coriander oil.

Another embodiment comprises cyclosporin A, Lonzest STS-20®, and Coriander oil.

Another embodiment comprises cyclosporin A, Lumulse GR-40®, and Coriander oil.

Another embodiment comprises cyclosporin A, Lumulse GRH-40®, and Coriander oil.

Another embodiment comprises cyclosporin A, Lumulse POE (7) GML®, and Coriander oil.

Another embodiment comprises cyclosporin A, Lumulse POE (12) Glyc®, and Coriander oil.

Another embodiment comprises cyclosporin A, Lumulse POE (40) MS KP®, and Coriander oil.

Another embodiment comprises cyclosporin A, Marlowet 4750®, and Coriander oil.

Another embodiment comprises cyclosporin A, Marlowet LVS®, and Coriander oil.

Another embodiment comprises cyclosporin A, Marlowet R 11®, and Coriander oil.

Another embodiment comprises cyclosporin A, Marlowet R 40®, and Coriander oil.

Another embodiment comprises cyclosporin A, Mazol 80 MGK®, and Coriander oil.

Another embodiment comprises cyclosporin A, Nonionic Emulsifier T-9®, and Coriander oil.

Another embodiment comprises cyclosporin A, Oronal LCG®, and Coriander oil.

Another embodiment comprises cyclosporin A, Polyderm PPI-CO-200®, and Coriander oil.

Another embodiment comprises cyclosporin A, Polyderm PPI-CO-40®, and Coriander oil.

Another embodiment comprises cyclosporin A, Rewoderm LI 520-70®, and Coriander oil.

Another embodiment comprises cyclosporin A, Ritapeg 150 DS®, and Coriander oil.

Another embodiment comprises cyclosporin A, Softigen 767®, and Coriander oil.

Another embodiment comprises cyclosporin A, Surfactol 318®, and Coriander oil.

Another embodiment comprises cyclosporin A, Surfactol 365®, and Coriander oil.

Another embodiment comprises cyclosporin A, Syn Lube 107®, and Coriander oil.

Another embodiment comprises cyclosporin A, Syn Lube 728®, and Coriander oil.

Another embodiment comprises cyclosporin A, Syn Lube 1632H®, and Coriander oil.

Another embodiment comprises cyclosporin A, Syn Lube 6277-A®, and Coriander oil.

Another embodiment comprises cyclosporin A, T-Det C-20®, and Coriander oil.

Another embodiment comprises cyclosporin A, T-Det C-40®, and Coriander oil.

Another embodiment comprises cyclosporin A, Tally 100 Plus®, and Coriander oil.

Another embodiment comprises cyclosporin A, Uniperol EL®, and Coriander oil.

Another embodiment comprises cyclosporin A, a Fatty Ester, and Coriander oil.

Another embodiment comprises cyclosporin A, Actralube-Syn 147®, and Coriander oil.

Another embodiment comprises cyclosporin A, Atlas G-1556®, and Coriander oil.

Another embodiment comprises cyclosporin A, Atlas G-1564®, and Coriander oil.

Another embodiment comprises cyclosporin A, Atlasol Base Oil S®, and Coriander oil.

Another embodiment comprises cyclosporin A, Base ML®, and Coriander oil.

Another embodiment comprises cyclosporin A, Base MT®, and Coriander oil.

Another embodiment comprises cyclosporin A, Cerasynt 303®, and Coriander oil.

Another embodiment comprises cyclosporin A, Dermol 1012®, and Coriander oil.

Another embodiment comprises cyclosporin A, Kemester 4000®, and Coriander oil.

Another embodiment comprises cyclosporin A, Lactipol S®, and Coriander oil.

Another embodiment comprises cyclosporin A, Magrabar PGO®, and Coriander oil.

Another embodiment comprises cyclosporin A, Mayco Base BFO®, and Coriander oil.

Another embodiment comprises cyclosporin A, Methyl Linoleate®, and Coriander oil.

Another embodiment comprises cyclosporin A, Pationic 122A®, and Coriander oil.

Another embodiment comprises cyclosporin A, Pationic 138C®, and Coriander oil.

Another embodiment comprises cyclosporin A, Pationic CSL®, and Coriander oil.

Another embodiment comprises cyclosporin A, Pationic ISL®, and Coriander oil.

Another embodiment comprises cyclosporin A, Pationic SBL®, and Coriander oil.

Another embodiment comprises cyclosporin A, Pationic SSL®, and Coriander oil.

Another embodiment comprises cyclosporin A, Ritasol®, and Coriander oil.

Another embodiment comprises cyclosporin A, Tego Alkanol CS 20®, and Coriander oil.

Another embodiment comprises cyclosporin A, Tego Alkanol L23 P®, and Coriander oil.

Another embodiment comprises cyclosporin A, Tego Alkanol S2®, and Coriander oil.

Another embodiment comprises cyclosporin A, Tego Alkanol S20 P®, and Coriander oil.

Another embodiment comprises cyclosporin A, Triemulsifier 600 MS®, and Coriander oil.

Another embodiment comprises cyclosporin A, a Glycerol Esters, and Coriander oil.

Another embodiment comprises cyclosporin A, Agro #9 Wint SBO®, and Coriander oil.

Another embodiment comprises cyclosporin A, Ahcovel Base 700®, and Coriander oil.

Another embodiment comprises cyclosporin A, Aldo HMS FG®, and Coriander oil.

Another embodiment comprises cyclosporin A, Aldo MLD®, and Coriander oil.

Another embodiment comprises cyclosporin A, Aldo MLD FG®, and Coriander oil.

Another embodiment comprises cyclosporin A, Aldo MO FG®, and Coriander oil.

Another embodiment comprises cyclosporin A, Aldo MS®, and Coriander oil.

Another embodiment comprises cyclosporin A, Aldo MS FG®, and Coriander oil.

Another embodiment comprises cyclosporin A, Aldo MS LG FG®, and Coriander oil.

Another embodiment comprises cyclosporin A, Aldo MSD®, and Coriander oil.

Another embodiment comprises cyclosporin A, Aldo MSD FG®, and Coriander oil.

Another embodiment comprises cyclosporin A, Aldosperse O-20 FG®, and Coriander oil.

Another embodiment comprises cyclosporin A, Aldosperse TS-20 FG®, and Coriander oil.

Another embodiment comprises cyclosporin A, Aldosperse TS-40 FG®, and Coriander oil.

Another embodiment comprises cyclosporin A, Arlacel 165®, and Coriander oil.

Another embodiment comprises cyclosporin A, Arlacel 186®, and Coriander oil.

Another embodiment comprises cyclosporin A, Capmul GMO®, and Coriander oil.

Another embodiment comprises cyclosporin A, Capmul GMS®, and Coriander oil.

Another embodiment comprises cyclosporin A, Caprol 3GO®, and Coriander oil.

Another embodiment comprises cyclosporin A, Caprol 3GVS®, and Coriander oil.

Another embodiment comprises cyclosporin A, Caprol 6G2S®, and Coriander oil.

Another embodiment comprises cyclosporin A, Caprol 10G40®, and Coriander oil.

Another embodiment comprises cyclosporin A, Caprol 10G100®, and Coriander oil.

Another embodiment comprises cyclosporin A, Caprol ET®, and Coriander oil.

Another embodiment comprises cyclosporin A, Caprol PGE860®, and Coriander oil.

Another embodiment comprises cyclosporin A, Cerasynt 945®, and Coriander oil.

Another embodiment comprises cyclosporin A, Cerasynt GMS®, and Coriander oil.

Another embodiment comprises cyclosporin A, Cerasynt Q®, and Coriander oil.

Another embodiment comprises cyclosporin A, Cerasynt SD®, and Coriander oil.

Another embodiment comprises cyclosporin A, Cerasynt WM®, and Coriander oil.

Another embodiment comprises cyclosporin A, Chemsperse 14®, and Coriander oil.

Another embodiment comprises cyclosporin A, Cremophor GO-32®, and Coriander oil.

Another embodiment comprises cyclosporin A, Cremophor GS11®, and Coriander oil.

Another embodiment comprises cyclosporin A, Cremophor GS-32®, and Coriander oil.

Another embodiment comprises cyclosporin A, Cutina KD-16®, and Coriander oil.

Another embodiment comprises cyclosporin A, Dehymuls PGPH®, and Coriander oil.

Another embodiment comprises cyclosporin A, Dermol DGDIS®, and Coriander oil.

Another embodiment comprises cyclosporin A, Dermol DGMIS®, and Coriander oil.

Another embodiment comprises cyclosporin A, Dermol G-76®, and Coriander oil.

Another embodiment comprises cyclosporin A, Dermol G-7DI®, and Coriander oil.

Another embodiment comprises cyclosporin A, Dermol NGDI®, and Coriander oil.

Another embodiment comprises cyclosporin A, Dermolan GLH®, and Coriander oil.

Another embodiment comprises cyclosporin A, Drewmulse GMO®, and Coriander oil.

Another embodiment comprises cyclosporin A, Drewpol 3-5-M®, and Coriander oil.

Another embodiment comprises cyclosporin A, Durlac 100 W®, and Coriander oil.

Another embodiment comprises cyclosporin A, Dur-Lo®, and Coriander oil.

Another embodiment comprises cyclosporin A, Dynasan 118®, and Coriander oil.

Another embodiment comprises cyclosporin A, EC-25®, and Coriander oil.

Another embodiment comprises cyclosporin A, EM 40®, and Coriander oil.

Another embodiment comprises cyclosporin A, Emerest 2400®, and Coriander oil.

Another embodiment comprises cyclosporin A, Emerest 2452®, and Coriander oil.

Another embodiment comprises cyclosporin A, Empilan G-26®, and Coriander oil.

Another embodiment comprises cyclosporin A, Genapol TSM®, and Coriander oil.

Another embodiment comprises cyclosporin A, Hostacerin DGI®, and Coriander oil.

Another embodiment comprises cyclosporin A, Hostacerin DGL®, and Coriander oil.

Another embodiment comprises cyclosporin A, Hostacerin DGMS®, and Coriander oil.

Another embodiment comprises cyclosporin A, Hostacerin DGSB®, and Coriander oil.

Another embodiment comprises cyclosporin A, Ice No. 2®, and Coriander oil.

Another embodiment comprises cyclosporin A, Imwitor 742®, and Coriander oil.

Another embodiment comprises cyclosporin A, Imwitor 780 K®, and Coriander oil.

Another embodiment comprises cyclosporin A, Imwitor 960 Flakes®, and Coriander oil.

Another embodiment comprises cyclosporin A, Isolan GI 34®, and Coriander oil.

Another embodiment comprises cyclosporin A, Isolan GO 33®, and Coriander oil.

Another embodiment comprises cyclosporin A, Kemester 1000®, and Coriander oil.

Another embodiment comprises cyclosporin A, Kemester 2000®, and Coriander oil.

Another embodiment comprises cyclosporin A, Kemester 2000®, and Coriander oil.

Another embodiment comprises cyclosporin A, Kemester 6000SE®, and Coriander oil.

Another embodiment comprises cyclosporin A, Lamecreme DGE 18®, and Coriander oil.

Another embodiment comprises cyclosporin A, Lexemul 515®, and Coriander oil.

Another embodiment comprises cyclosporin A, Lexemul 561®, and Coriander oil.

Another embodiment comprises cyclosporin A, Lexemul AR®, and Coriander oil.

Another embodiment comprises cyclosporin A, Lexemul AS®, and Coriander oil.

Another embodiment comprises cyclosporin A, Lexemul GDL®, and Coriander oil.

Another embodiment comprises cyclosporin A, Lexemul T®, and Coriander oil.

Another embodiment comprises cyclosporin A, Lipomulse 165®, and Coriander oil.

Another embodiment comprises cyclosporin A, Lumulse GML K®, and Coriander oil.

Another embodiment comprises cyclosporin A, Lumulse GMO K®, and Coriander oil.

Another embodiment comprises cyclosporin A, Lumulse GMR K®, and Coriander oil.

Another embodiment comprises cyclosporin A, Lumulse GMT K®, and Coriander oil.

Another embodiment comprises cyclosporin A, Magrabar GMC®, and Coriander oil.

Another embodiment comprises cyclosporin A, Magrabar GMO-CK®, and Coriander oil.

Another embodiment comprises cyclosporin A, Magrabar MDG-5050®, and Coriander oil.

Another embodiment comprises cyclosporin A, Magrabar PGO-315®, and Coriander oil.

Another embodiment comprises cyclosporin A, Magrabar PGO-1010®, and Coriander oil.

Another embodiment comprises cyclosporin A, Mazol 300K®, and Coriander oil.

Another embodiment comprises cyclosporin A, Mazol GMO-K®, and Coriander oil.

Another embodiment comprises cyclosporin A, Mazol GMS-K®, and Coriander oil.

Another embodiment comprises cyclosporin A, Mazol PG031-K®, and Coriander oil.

Another embodiment comprises cyclosporin A, Miglyol 812®, and Coriander oil.

Another embodiment comprises cyclosporin A, Norfox 165C®, and Coriander oil.

Another embodiment comprises cyclosporin A, Schercemol GMIS®, and Coriander oil.

Another embodiment comprises cyclosporin A, Tegin®, and Coriander oil.

Another embodiment comprises cyclosporin A, Tegin 4100 Pellets®, and Coriander oil.

Another embodiment comprises cyclosporin A, Tegin M Pellets®, and Coriander oil.

Another embodiment comprises cyclosporin A, Tegin OV®, and Coriander oil.

Another embodiment comprises cyclosporin A, Teginacid H®, and Coriander oil.

Another embodiment comprises cyclosporin A, Tego Cosmo P813®, and Coriander oil.

Another embodiment comprises cyclosporin A, Wickenol 535®, and Coriander oil.

Another embodiment comprises cyclosporin A, Witconol 14®, and Coriander oil.

Another embodiment comprises cyclosporin A, Witconol 14®, and Coriander oil.

Another embodiment comprises cyclosporin A, Witconol 14F®, and Coriander oil.

Another embodiment comprises cyclosporin A, Witconol 18L®, and Coriander oil.

Another embodiment comprises cyclosporin A, Witconol GOT®, and Coriander oil.

Another embodiment comprises cyclosporin A, Witconol MST®, and Coriander oil.

Another embodiment comprises cyclosporin A, Witconol RHT®, and Coriander oil.

Another embodiment comprises cyclosporin A, a Glycol Ester, and Coriander oil.

Another embodiment comprises cyclosporin A, Alkamuls 600 DO®, and Coriander oil.

Another embodiment comprises cyclosporin A, Alkamuls SEG®, and Coriander oil.

Another embodiment comprises cyclosporin A, Atlas EM-2®, and Coriander oil.

Another embodiment comprises cyclosporin A, Cerasynt IP®, and Coriander oil.

Another embodiment comprises cyclosporin A, Cerasynt M®, and Coriander oil.

Another embodiment comprises cyclosporin A, Cerasynt MN®, and Coriander oil.

Another embodiment comprises cyclosporin A, Cerasynt PA®, and Coriander oil.

Another embodiment comprises cyclosporin A, Chemsperse EGDS®, and Coriander oil.

Another embodiment comprises cyclosporin A, Chemsperse EGMS®, and Coriander oil.

Another embodiment comprises cyclosporin A, Colonial Monolaurin®, and Coriander oil.

Another embodiment comprises cyclosporin A, DeMuls SGE-95®, and Coriander oil.

Another embodiment comprises cyclosporin A, Eccoterge 200®, and Coriander oil.

Another embodiment comprises cyclosporin A, Emerest 2380®, and Coriander oil.

Another embodiment comprises cyclosporin A, Ethox 2610®, and Coriander oil.

Another embodiment comprises cyclosporin A, Ethox DO-9®, and Coriander oil.

Another embodiment comprises cyclosporin A, Ethox DO-14®, and Coriander oil.

Another embodiment comprises cyclosporin A, Ethox SO-9®, and Coriander oil.

Another embodiment comprises cyclosporin A, Fizul MD-318®, and Coriander oil.

Another embodiment comprises cyclosporin A, Genapol EGDS-VHP®, and Coriander oil.

Another embodiment comprises cyclosporin A, Genapol TS Powder®, and Coriander oil.

Another embodiment comprises cyclosporin A, Hostacerin WO®, and Coriander oil.

Another embodiment comprises cyclosporin A, Inversol 140®, and Coriander oil.

Another embodiment comprises cyclosporin A, Kemester 104®, and Coriander oil.

Another embodiment comprises cyclosporin A, Kemester 205®, and Coriander oil.

Another embodiment comprises cyclosporin A, Kemester 226®, and Coriander oil.

Another embodiment comprises cyclosporin A, Kemester 5221SE®, and Coriander oil.

Another embodiment comprises cyclosporin A, Kemester EGDS®, and Coriander oil.

Another embodiment comprises cyclosporin A, Lexemul EGDS®, and Coriander oil.

Another embodiment comprises cyclosporin A, Lexemul EGMS®, and Coriander oil.

Another embodiment comprises cyclosporin A, Lexemul P®, and Coriander oil.

Another embodiment comprises cyclosporin A, Lipo DGLS®, and Coriander oil.

Another embodiment comprises cyclosporin A, Lipo EGDS®, and Coriander oil.

Another embodiment comprises cyclosporin A, Lipo PGMS®, and Coriander oil.

Another embodiment comprises cyclosporin A, Liposorb S-4®, and Coriander oil.

Another embodiment comprises cyclosporin A, Liposorb TO-20®, and Coriander oil.

Another embodiment comprises cyclosporin A, Lumulse PGO®, and Coriander oil.

Another embodiment comprises cyclosporin A, Mackester EGDS®, and Coriander oil.

Another embodiment comprises cyclosporin A, Mackester EGMS®, and Coriander oil.

Another embodiment comprises cyclosporin A, Mackester GSTP®, and Coriander oil.

Another embodiment comprises cyclosporin A, Mackester Series®, and Coriander oil.

Another embodiment comprises cyclosporin A, Magrabar PDG-50®, and Coriander oil.

Another embodiment comprises cyclosporin A, Mapeg 6000 DS®, and Coriander oil.

Another embodiment comprises cyclosporin A, Marlowet 4702®, and Coriander oil.

Another embodiment comprises cyclosporin A, Monalube 305®, and Coriander oil.

Another embodiment comprises cyclosporin A, Monalube 310®, and Coriander oil.

Another embodiment comprises cyclosporin A, Monalube 315®, and Coriander oil.

Another embodiment comprises cyclosporin A, Monalube 320®, and Coriander oil.

Another embodiment comprises cyclosporin A, Monalube 325®, and Coriander oil.

Another embodiment comprises cyclosporin A, Monalube 330®, and Coriander oil.

Another embodiment comprises cyclosporin A, Naturechem PGHS®, and Coriander oil.

Another embodiment comprises cyclosporin A, Polycastorol PLO-840®, and Coriander oil.

Another embodiment comprises cyclosporin A, Polytex 10M®, and Coriander oil.

Another embodiment comprises cyclosporin A, Ritasynt IP®, and Coriander oil.

Another embodiment comprises cyclosporin A, Ross Chem PEG 600 DT®, and Coriander oil.

Another embodiment comprises cyclosporin A, Schercemol PGMS®, and Coriander oil.

Another embodiment comprises cyclosporin A, Sponto H-44C®, and Coriander oil.

Another embodiment comprises cyclosporin A, Tegin G®, and Coriander oil.

Another embodiment comprises cyclosporin A, Witbreak DGE-182®, and Coriander oil.

Another embodiment comprises cyclosporin A, Witbreak DGE-182®, and Coriander oil.

Another embodiment comprises cyclosporin A, Witbreak DRA-21®, and Coriander oil.

Another embodiment comprises cyclosporin A, Witbreak DRA-21®, and Coriander oil.

Another embodiment comprises cyclosporin A, Witbreak DRA-50®, and Coriander oil.

Another embodiment comprises cyclosporin A, Witbreak DRA-50®, and Coriander oil.

Another embodiment comprises cyclosporin A, Witconol F26-46®, and Coriander oil.

Another embodiment comprises cyclosporin A, Witconol H-32®, and Coriander oil.

Another embodiment comprises cyclosporin A, Witconol H-33®, and Coriander oil.

Another embodiment comprises cyclosporin A, Witconol H-35A®, and Coriander oil.

Another embodiment comprises cyclosporin A, Witconol RHP®, and Coriander oil.

Another embodiment comprises cyclosporin A, a Lanolin-based Derivative, and Coriander oil.

Another embodiment comprises cyclosporin A, Amerchol CAB®, and Coriander oil.

Another embodiment comprises cyclosporin A, Amerchol L-101®, and Coriander oil.

Another embodiment comprises cyclosporin A, Amerlate LFA-LO®, and Coriander oil.

Another embodiment comprises cyclosporin A, Amerlate P®, and Coriander oil.

Another embodiment comprises cyclosporin A, Barre Common Degras®, and Coriander oil.

Another embodiment comprises cyclosporin A, Cholesterol NF®, and Coriander oil.

Another embodiment comprises cyclosporin A, Crodalan AWS®, and Coriander oil.

Another embodiment comprises cyclosporin A, Crodalan LA®, and Coriander oil.

Another embodiment comprises cyclosporin A, Emery 1650®, and Coriander oil.

Another embodiment comprises cyclosporin A, Emery 1650®, and Coriander oil.

Another embodiment comprises cyclosporin A, Emery 1740®, and Coriander oil.

Another embodiment comprises cyclosporin A, Emery 1740®, and Coriander oil.

Another embodiment comprises cyclosporin A, Forlan 500®, and Coriander oil.

Another embodiment comprises cyclosporin A, Forlan L®, and Coriander oil.

Another embodiment comprises cyclosporin A, Laneto 50®, and Coriander oil.

Another embodiment comprises cyclosporin A, Laneto 100®, and Coriander oil.

Another embodiment comprises cyclosporin A, Laneto AWS®, and Coriander oil.

Another embodiment comprises cyclosporin A, Lanfrax 1776®, and Coriander oil.

Another embodiment comprises cyclosporin A, Lanfrax 1776®, and Coriander oil.

Another embodiment comprises cyclosporin A, Lanogel 21®, and Coriander oil.

Another embodiment comprises cyclosporin A, Lipolan®, and Coriander oil.

Another embodiment comprises cyclosporin A, Lipolan 31®, and Coriander oil.

Another embodiment comprises cyclosporin A, OHlan®, and Coriander oil.

Another embodiment comprises cyclosporin A, Polychol 5®, and Coriander oil.

Another embodiment comprises cyclosporin A, Polychol 15®, and Coriander oil.

Another embodiment comprises cyclosporin A, Ritacetyl®, and Coriander oil.

Another embodiment comprises cyclosporin A, Ritachol®, and Coriander oil.

Another embodiment comprises cyclosporin A, Ritahydrox®, and Coriander oil.

Another embodiment comprises cyclosporin A, Ritalafa®, and Coriander oil.

Another embodiment comprises cyclosporin A, Ritalan®, and Coriander oil.

Another embodiment comprises cyclosporin A, Ritalan AWS®, and Coriander oil.

Another embodiment comprises cyclosporin A, Ritalan C®, and Coriander oil.

Another embodiment comprises cyclosporin A, Ritawax®, and Coriander oil.

Another embodiment comprises cyclosporin A, Ritawax AEO®, and Coriander oil.

Another embodiment comprises cyclosporin A, Ritawax ALA®, and Coriander oil.

Another embodiment comprises cyclosporin A, Solan/Solan 50/Super Solan®, and Coriander oil.

Another embodiment comprises cyclosporin A, Super Hartolan/Hartolan®, and Coriander oil.

Another embodiment comprises cyclosporin A, Supersat AWS-4®, and Coriander oil.

Another embodiment comprises cyclosporin A, Supersat AWS-24®, and Coriander oil.

Another embodiment comprises cyclosporin A, a Lecithin or Lecithin Derivative, and Coriander oil.

Another embodiment comprises cyclosporin A, Alcolec®, and Coriander oil.

Another embodiment comprises cyclosporin A, Lecithin®, and Coriander oil.

Another embodiment comprises cyclosporin A, Lexin K®, and Coriander oil.

Another embodiment comprises cyclosporin A, Natipide®, and Coriander oil.

Another embodiment comprises cyclosporin A, a Lignin or Lignin Derivative, and Coriander oil.

Another embodiment comprises cyclosporin A, Diwatex XP 9®, and Coriander oil.

Another embodiment comprises cyclosporin A, Dynasperse LCD®, and Coriander oil.

Another embodiment comprises cyclosporin A, Indulin SAL®, and Coriander oil.

Another embodiment comprises cyclosporin A, Indulin W-1®, and Coriander oil.

Another embodiment comprises cyclosporin A, lndulin W-5®, and Coriander oil.

Another embodiment comprises cyclosporin A, Lignosol FTA®, and Coriander oil.

Another embodiment comprises cyclosporin A, Lignosol SFX-65®, and Coriander oil.

Another embodiment comprises cyclosporin A, Marasperse 52 CP®, and Coriander oil.

Another embodiment comprises cyclosporin A, Marasperse AG®, and Coriander oil.

Another embodiment comprises cyclosporin A, Marasperse CBOS-4®, and Coriander oil.

Another embodiment comprises cyclosporin A, Ufoxane 2®, and Coriander oil.

Another embodiment comprises cyclosporin A, a Methyl Ester, and Coriander oil.

Another embodiment comprises cyclosporin A, E.B. Cleaner AK®, and Coriander oil.

Another embodiment comprises cyclosporin A, Oleocal ME-70®, and Coriander oil.

Another embodiment comprises cyclosporin A, Trade Name Company®, and Coriander oil.

Another embodiment comprises cyclosporin A, Methyl Esters (cont'd)®, and Coriander oil.

Another embodiment comprises cyclosporin A, Oleocal ME-92®, and Coriander oil.

Another embodiment comprises cyclosporin A, Oleocal ME-112®, and Coriander oil.

Another embodiment comprises cyclosporin A, Oleocal ME-130®, and Coriander oil.

Another embodiment comprises cyclosporin A, a Monoglyceride or a Derivative thereof, and Coriander oil.

Another embodiment comprises cyclosporin A, Dynacet 211®, and Coriander oil.

Another embodiment comprises cyclosporin A, Hetsorb S-20®, and Coriander oil.

Another embodiment comprises cyclosporin A, Imwitor 191®, and Coriander oil.

Another embodiment comprises cyclosporin A, Imwitor 370®, and Coriander oil.

Another embodiment comprises cyclosporin A, Imwitor 375®, and Coriander oil.

Another embodiment comprises cyclosporin A, Imwitor 900®, and Coriander oil.

Another embodiment comprises cyclosporin A, Imwitor 945®, and Coriander oil.

Another embodiment comprises cyclosporin A, Imwitor 2020®, and Coriander oil.

Another embodiment comprises cyclosporin A, Kemester 5500®, and Coriander oil.

Another embodiment comprises cyclosporin A, Kemester 6000®, and Coriander oil.

Another embodiment comprises cyclosporin A, Magrabar GMC®, and Coriander oil.

Another embodiment comprises cyclosporin A, Magrabar GMO-CK®, and Coriander oil.

Another embodiment comprises cyclosporin A, Magrabar GPC-10®, and Coriander oil.

Another embodiment comprises cyclosporin A, Magrabar MDG-5050®, and Coriander oil.

Another embodiment comprises cyclosporin A, Monalube 335®, and Coriander oil.

Another embodiment comprises cyclosporin A, Monoglycerides Glyceryl Monestearate Archer Daniels Midland Company®, and Coriander oil.

Another embodiment comprises cyclosporin A, Rita GMS®, and Coriander oil.

Another embodiment comprises cyclosporin A, Ritamulse SCG®, and Coriander oil.

Another embodiment comprises cyclosporin A, Softigen 701®, and Coriander oil.

Another embodiment comprises cyclosporin A, Tally 100 Plus®, and Coriander oil.

Another embodiment comprises cyclosporin A, a Polyethylene Glycol, and Coriander oil.

Another embodiment comprises cyclosporin A, Emulgade PL 68/50®, and Coriander oil.

Another embodiment comprises cyclosporin A, Lumulse PEG®, and Coriander oil.

Another embodiment comprises cyclosporin A, Rhodasurf PEG-400®, and Coriander oil.

Another embodiment comprises cyclosporin A, Rhodasurf PEG-600®, and Coriander oil.

Another embodiment comprises cyclosporin A, Witconol PEG-400®, and Coriander oil.

Another embodiment comprises cyclosporin A, a Polymeric Surfactant, and Coriander oil.

Another embodiment comprises cyclosporin A, Acritamer PNC-EG®, and Coriander oil.

Another embodiment comprises cyclosporin A, Ag-Rho DEP-775®, and Coriander oil.

Another embodiment comprises cyclosporin A, APG 325N Glycoside®, and Coriander oil.

Another embodiment comprises cyclosporin A, Aristoflex AVC®, and Coriander oil.

Another embodiment comprises cyclosporin A, Aristoflex HMB®, and Coriander oil.

Another embodiment comprises cyclosporin A, Burco NPS-225®, and Coriander oil.

Another embodiment comprises cyclosporin A, Burco NPS-816®, and Coriander oil.

Another embodiment comprises cyclosporin A, Chemccinate 5603®, and Coriander oil.

Another embodiment comprises cyclosporin A, Cosmedia Guar C-261N®, and Coriander oil.

Another embodiment comprises cyclosporin A, Gantrez S-95®, and Coriander oil.

Another embodiment comprises cyclosporin A, Glucopon 220 UP®, and Coriander oil.

Another embodiment comprises cyclosporin A, Glucopon 225 DK®, and Coriander oil.

Another embodiment comprises cyclosporin A, Glucopon 425 N®, and Coriander oil.

Another embodiment comprises cyclosporin A, Glucopon 600 UP®, and Coriander oil.

Another embodiment comprises cyclosporin A, Glucopon 625 UP®, and Coriander oil.

Another embodiment comprises cyclosporin A, Pemulen 1621®, and Coriander oil.

Another embodiment comprises cyclosporin A, Pemulen 1622®, and Coriander oil.

Another embodiment comprises cyclosporin A, Pemulen TR-1®, and Coriander oil.

Another embodiment comprises cyclosporin A, Pemulen TR-2®, and Coriander oil.

Another embodiment comprises cyclosporin A, Plantacare 818®, and Coriander oil.

Another embodiment comprises cyclosporin A, Plantapon LGC Sorb®, and Coriander oil.

Another embodiment comprises cyclosporin A, Plantaren 1200N®, and Coriander oil.

Another embodiment comprises cyclosporin A, Plantaren 2000N®, and Coriander oil.

Another embodiment comprises cyclosporin A, Viscolam AT 64®, and Coriander oil.

Another embodiment comprises cyclosporin A, Viscolam AT 64P®, and Coriander oil.

Another embodiment comprises cyclosporin A, Viscolam AT 100®, and Coriander oil.

Another embodiment comprises cyclosporin A, Viscolam MAC 7®, and Coriander oil.

Another embodiment comprises cyclosporin A, Viscolam SMC 20®, and Coriander oil.

Another embodiment comprises cyclosporin A, Witbreak RTC-323®, and Coriander oil.

Another embodiment comprises cyclosporin A, WSI 3700®, and Coriander oil.

Another embodiment comprises cyclosporin A, a Propoxylated or Ethoxylated Fatty Acid, Alcohol, or Alkyl Phenol, and Coriander oil.

Another embodiment comprises cyclosporin A, Antarox AA-60®, and Coriander oil.

Another embodiment comprises cyclosporin A, Antarox LF-224®, and Coriander oil.

Another embodiment comprises cyclosporin A, Burcomul DFE-45®, and Coriander oil.

Another embodiment comprises cyclosporin A, Burcoterge LFE-1000®, and Coriander oil.

Another embodiment comprises cyclosporin A, Chemal LF-25B®, and Coriander oil.

Another embodiment comprises cyclosporin A, Chemal LF-40B®, and Coriander oil.

Another embodiment comprises cyclosporin A, Dehypon LS-36®, and Coriander oil.

Another embodiment comprises cyclosporin A, Dehypon LS-36®, and Coriander oil.

Another embodiment comprises cyclosporin A, Dehypon LS-54®, and Coriander oil.

Another embodiment comprises cyclosporin A, Dehypon LS-54®, and Coriander oil.

Another embodiment comprises cyclosporin A, Delonic 100 VLF®, and Coriander oil.

Another embodiment comprises cyclosporin A, Delonic LF-60 MOD®, and Coriander oil.

Another embodiment comprises cyclosporin A, Empiderm B®, and Coriander oil.

Another embodiment comprises cyclosporin A, Ethylan 1206®, and Coriander oil.

Another embodiment comprises cyclosporin A, Ethylan NS-500K®, and Coriander oil.

Another embodiment comprises cyclosporin A, Ethylan NS-500LQ®, and Coriander oil.

Another embodiment comprises cyclosporin A, Genapol 1392®, and Coriander oil.

Another embodiment comprises cyclosporin A, Genapol 2317®, and Coriander oil.

Another embodiment comprises cyclosporin A, Genapol 26EP710®, and Coriander oil.

Another embodiment comprises cyclosporin A, Genapol EP 1022®, and Coriander oil.

Another embodiment comprises cyclosporin A, Genapol EP 1024®, and Coriander oil.

Another embodiment comprises cyclosporin A, Genapol EP 6068®, and Coriander oil.

Another embodiment comprises cyclosporin A, Genapol NP915®, and Coriander oil.

Another embodiment comprises cyclosporin A, Kieralon MFB®, and Coriander oil.

Another embodiment comprises cyclosporin A, Lumisolve CSA-80 V®, and Coriander oil.

Another embodiment comprises cyclosporin A, Marlowet 5001®, and Coriander oil.

Another embodiment comprises cyclosporin A, Marlox FK 64®, and Coriander oil.

Another embodiment comprises cyclosporin A, Marlox MO 124®, and Coriander oil.

Another embodiment comprises cyclosporin A, Marlox S 58®, and Coriander oil.

Another embodiment comprises cyclosporin A, Nonatell 1003®, and Coriander oil.

Another embodiment comprises cyclosporin A, Nonatell 1038®, and Coriander oil.

Another embodiment comprises cyclosporin A, Nonatell 1052®, and Coriander oil.

Another embodiment comprises cyclosporin A, Nonatell 1061®, and Coriander oil.

Another embodiment comprises cyclosporin A, Nonatell 1075®, and Coriander oil.

Another embodiment comprises cyclosporin A, Nonatell 1088®, and Coriander oil.

Another embodiment comprises cyclosporin A, Nonatell 1123®, and Coriander oil.

Another embodiment comprises cyclosporin A, Nonatell 1153®, and Coriander oil.

Another embodiment comprises cyclosporin A, Nonatell 1161®, and Coriander oil.

Another embodiment comprises cyclosporin A, Nonatell 1172®, and Coriander oil.

Another embodiment comprises cyclosporin A, Nonatell 1181®, and Coriander oil.

Another embodiment comprises cyclosporin A, Norfox 36®, and Coriander oil.

Another embodiment comprises cyclosporin A, Procetyl AWS®, and Coriander oil.

Another embodiment comprises cyclosporin A, Sandoxylate SX 412®, and Coriander oil.

Another embodiment comprises cyclosporin A, Sandoxylate SX 418®, and Coriander oil.

Another embodiment comprises cyclosporin A, Surfonic JL-80X®, and Coriander oil.

Another embodiment comprises cyclosporin A, Surfonic JL-80X-B1®, and Coriander oil.

Another embodiment comprises cyclosporin A, Surfonic L4-29X®, and Coriander oil.

Another embodiment comprises cyclosporin A, Surfonic LF®, and Coriander oil.

Another embodiment comprises cyclosporin A, T-Det A826®, and Coriander oil.

Another embodiment comprises cyclosporin A, T-Det LF-416®, and Coriander oil.

Another embodiment comprises cyclosporin A, Tergitol Min-Foam 1X®, and Coriander oil.

Another embodiment comprises cyclosporin A, Tergitol Min-Foam 2X®, and Coriander oil.

Another embodiment comprises cyclosporin A, Triton CF-21®, and Coriander oil.

Another embodiment comprises cyclosporin A, Triton CF-76®, and Coriander oil.

Another embodiment comprises cyclosporin A, Triton XL-80N®, and Coriander oil.

Another embodiment comprises cyclosporin A, Witconol NS-98®, and Coriander oil.

Another embodiment comprises cyclosporin A, Witconol NS-108LQ®, and Coriander oil.

Another embodiment comprises cyclosporin A, Witconol NS-145®, and Coriander oil.

Another embodiment comprises cyclosporin A, Witconol NS-179®, and Coriander oil.

Another embodiment comprises cyclosporin A, a Protein-based Surfactant, and Coriander oil.

Another embodiment comprises cyclosporin A, AminoFoam W®, and Coriander oil.

Another embodiment comprises cyclosporin A, Amiter LGOD-2®, and Coriander oil.

Another embodiment comprises cyclosporin A, Amiter LGS-2®, and Coriander oil.

Another embodiment comprises cyclosporin A, Amiter LGS-5®, and Coriander oil.

Another embodiment comprises cyclosporin A, Lamepon S®, and Coriander oil.

Another embodiment comprises cyclosporin A, Lamepon S®, and Coriander oil.

Another embodiment comprises cyclosporin A, Maypon 4C®, and Coriander oil.

Another embodiment comprises cyclosporin A, May-Tein C®, and Coriander oil.

Another embodiment comprises cyclosporin A, May-Tein CT®, and Coriander oil.

Another embodiment comprises cyclosporin A, May-Tein KTS®, and Coriander oil.

Another embodiment comprises cyclosporin A, May-Tein SY®, and Coriander oil.

Another embodiment comprises cyclosporin A, Plantapon S®, and Coriander oil.

Another embodiment comprises cyclosporin A, Proteol APL®, and Coriander oil.

Another embodiment comprises cyclosporin A, Proteol OAT®, and Coriander oil.

Another embodiment comprises cyclosporin A, Pyroter CPI-40®, and Coriander oil.

Another embodiment comprises cyclosporin A, Pyroter GPI-25®, and Coriander oil.

Another embodiment comprises cyclosporin A, Supro-Tein S®, and Coriander oil.

Another embodiment comprises cyclosporin A, Supro-Tein V®, and Coriander oil.

Another embodiment comprises cyclosporin A,® and Coriander oil.

Another embodiment comprises cyclosporin A, a Sarcosine Derivative®, and Coriander oil.

Another embodiment comprises cyclosporin A, Crodasinic LS-30®, and Coriander oil.

Another embodiment comprises cyclosporin A, Vanseal CS®, and Coriander oil.

Another embodiment comprises cyclosporin A, Vanseal LS®, and Coriander oil.

Another embodiment comprises cyclosporin A, Vanseal MS®, and Coriander oil.

Another embodiment comprises cyclosporin A, Vanseal NACS-30®, and Coriander oil.

Another embodiment comprises cyclosporin A, Vanseal NALS-95®, and Coriander oil.

Another embodiment comprises cyclosporin A, Vanseal OS®, and Coriander oil.

Another embodiment comprises cyclosporin A,® and Coriander oil.

Another embodiment comprises cyclosporin A, a Silicone-based Surfactant®, and Coriander oil.

Another embodiment comprises cyclosporin A, Abil-B-9950®, and Coriander oil.

Another embodiment comprises cyclosporin A, Abil Care 85®, and Coriander oil.

Another embodiment comprises cyclosporin A, Abil EM 90®, and Coriander oil.

Another embodiment comprises cyclosporin A, Abil EM 97®, and Coriander oil.

Another embodiment comprises cyclosporin A, Abil WE-09®, and Coriander oil.

Another embodiment comprises cyclosporin A, Dow Corning 1248 Fluid®, and Coriander oil.

Another embodiment comprises cyclosporin A, Dow Corning 3225C®, and Coriander oil.

Another embodiment comprises cyclosporin A, Dow Corning 5200®, and Coriander oil.

Another embodiment comprises cyclosporin A, Dow Corning Q4-3667®, and Coriander oil.

Another embodiment comprises cyclosporin A, Monasil PCA®, and Coriander oil.

Another embodiment comprises cyclosporin A, Monasil PDM®, and Coriander oil.

Another embodiment comprises cyclosporin A, Monasil PLN®, and Coriander oil.

Another embodiment comprises cyclosporin A, Polyderm PPI-SI-WS®, and Coriander oil.

Another embodiment comprises cyclosporin A, Troysol 380W®, and Coriander oil.

Another embodiment comprises cyclosporin A, Troysol S366®, and Coriander oil.

Another embodiment comprises cyclosporin A, a Sorbitan Derivative, and Coriander oil.

Another embodiment comprises cyclosporin A, Alkamuls SML®, and Coriander oil.

Another embodiment comprises cyclosporin A, Alkamuls SMO®, and Coriander oil.

Another embodiment comprises cyclosporin A, Alkamuls STO®, and Coriander oil.

Another embodiment comprises cyclosporin A, Arlacel 20®, and Coriander oil.

Another embodiment comprises cyclosporin A, Arlacel 40®, and Coriander oil.

Another embodiment comprises cyclosporin A, Arlacel 60®, and Coriander oil.

Another embodiment comprises cyclosporin A, Arlacel 80®, and Coriander oil.

Another embodiment comprises cyclosporin A, Arlacel C®, and Coriander oil.

Another embodiment comprises cyclosporin A, Armul 21®, and Coriander oil.

Another embodiment comprises cyclosporin A, Atlox 80®, and Coriander oil.

Another embodiment comprises cyclosporin A, Atlox 847®, and Coriander oil.

Another embodiment comprises cyclosporin A, Atlox 1045A®, and Coriander oil.

Another embodiment comprises cyclosporin A, Canarcel 20®, and Coriander oil.

Another embodiment comprises cyclosporin A, Canarcel 60®, and Coriander oil.

Another embodiment comprises cyclosporin A, Canarcel 80®, and Coriander oil.

Another embodiment comprises cyclosporin A, Canarcel TW 20®, and Coriander oil.

Another embodiment comprises cyclosporin A, Canarcel TW 60®, and Coriander oil.

Another embodiment comprises cyclosporin A, Canarcel TW 80®, and Coriander oil.

Another embodiment comprises cyclosporin A, Coladet BSB-P®, and Coriander oil.

Another embodiment comprises cyclosporin A, Customulse O-20®, and Coriander oil.

Another embodiment comprises cyclosporin A, Dehymuls E®, and Coriander oil.

Another embodiment comprises cyclosporin A, DeSotan SMO®, and Coriander oil.

Another embodiment comprises cyclosporin A, DeSotan SMO-20®, and Coriander oil.

Another embodiment comprises cyclosporin A, DeSotan SMT®, and Coriander oil.

Another embodiment comprises cyclosporin A, DeSotan SMT-20®, and Coriander oil.

Another embodiment comprises cyclosporin A, Durfax 60®, and Coriander oil.

Another embodiment comprises cyclosporin A, Durfax 65®, and Coriander oil.

Another embodiment comprises cyclosporin A, Durfax 80®, and Coriander oil.

Another embodiment comprises cyclosporin A, Durtan 60®, and Coriander oil.

Another embodiment comprises cyclosporin A, Durtan 65®, and Coriander oil.

Another embodiment comprises cyclosporin A, Liposorb L®, and Coriander oil.

Another embodiment comprises cyclosporin A, Liposorb L-10®, and Coriander oil.

Another embodiment comprises cyclosporin A, Liposorb L-20®, and Coriander oil.

Another embodiment comprises cyclosporin A, Liposorb O®, and Coriander oil.

Another embodiment comprises cyclosporin A, Liposorb O-20®, and Coriander oil.

Another embodiment comprises cyclosporin A, Liposorb P®, and Coriander oil.

Another embodiment comprises cyclosporin A, Liposorb P-20®, and Coriander oil.

Another embodiment comprises cyclosporin A, Liposorb S®, and Coriander oil.

Another embodiment comprises cyclosporin A, Liposorb S-20®, and Coriander oil.

Another embodiment comprises cyclosporin A, Liposorb SQO®, and Coriander oil.

Another embodiment comprises cyclosporin A, Liposorb TO®, and Coriander oil.

Another embodiment comprises cyclosporin A, Liposorb TS®, and Coriander oil.

Another embodiment comprises cyclosporin A, Liposorb TS-20®, and Coriander oil.

Another embodiment comprises cyclosporin A, Lumisorb PS®, and Coriander oil.

Another embodiment comprises cyclosporin A, Lumisorb SMO (T)®, and Coriander oil.

Another embodiment comprises cyclosporin A, Lumisorb SMS K®, and Coriander oil.

Another embodiment comprises cyclosporin A, Lumisorb SSO®, and Coriander oil.

Another embodiment comprises cyclosporin A, Lumisorb STS K®, and Coriander oil.

Another embodiment comprises cyclosporin A, Lumisorb STT®, and Coriander oil.

Another embodiment comprises cyclosporin A, Magrabar SMO®, and Coriander oil.

Another embodiment comprises cyclosporin A, Magrabar SMO-VEG®, and Coriander oil.

Another embodiment comprises cyclosporin A, Magrabar SMT®, and Coriander oil.

Another embodiment comprises cyclosporin A, Magrabar STO®, and Coriander oil.

Another embodiment comprises cyclosporin A, Miracare BC-27®, and Coriander oil.

Another embodiment comprises cyclosporin A, Ritabate 20®, and Coriander oil.

Another embodiment comprises cyclosporin A, Ritabate 40®, and Coriander oil.

Another embodiment comprises cyclosporin A, Ritabate 60®, and Coriander oil.

Another embodiment comprises cyclosporin A, Ritabate 80®, and Coriander oil.

Another embodiment comprises cyclosporin A, T-Maz, and Coriander oil.

Another embodiment comprises cyclosporin A, Tego SML®, and Coriander oil.

Another embodiment comprises cyclosporin A, Tego SML 20®, and Coriander oil.

Another embodiment comprises cyclosporin A, Tego SMO 80 V®, and Coriander oil.

Another embodiment comprises cyclosporin A, Tego SMO V®, and Coriander oil.

Another embodiment comprises cyclosporin A, Tego SMS®, and Coriander oil.

Another embodiment comprises cyclosporin A, Tego STO V®, and Coriander oil.

Another embodiment comprises cyclosporin A, Tween 21®, and Coriander oil.

Another embodiment comprises cyclosporin A, Tween 40®, and Coriander oil.

Another embodiment comprises cyclosporin A, Tween 60®, and Coriander oil.

Another embodiment comprises cyclosporin A, Tween 60 K®, and Coriander oil.

Another embodiment comprises cyclosporin A, Tween 61®, and Coriander oil.

Another embodiment comprises cyclosporin A, Tween 65®, and Coriander oil.

Another embodiment comprises cycldsporin A, Tween 80®, and Coriander oil.

Another embodiment comprises cyclosporin A, Tween 80 K®, and Coriander oil.

Another embodiment comprises cyclosporin A, Tween 81®, and Coriander oil.

Another embodiment comprises cyclosporin A, Tween 85®, and Coriander oil.

Another embodiment comprises cyclosporin A, a Sucrose or Glucose Ester, or Derivative thereof, and Coriander oil.

Another embodiment comprises cyclosporin A, DeSulf GOS-P-60WCG®, and Coriander oil.

Another embodiment comprises cyclosporin A, Glucam E-20 Distearate®, and Coriander oil.

Another embodiment comprises cyclosporin A, Glucamate DOE-120®, and Coriander oil.

Another embodiment comprises cyclosporin A, Glucamate SSE-20®, and Coriander oil.

Another embodiment comprises cyclosporin A, Glucate DO®, and Coriander oil.

Another embodiment comprises cyclosporin A, Glucate SS®, and Coriander oil.

Another embodiment comprises cyclosporin A, Glucopon 425 UP®, and Coriander oil.

Another embodiment comprises cyclosporin A, Isolan IS®, and Coriander oil.

Another embodiment comprises cyclosporin A, Mazon 40®, and Coriander oil.

Another embodiment comprises cyclosporin A, Montanov 82®, and Coriander oil.

Another embodiment comprises cyclosporin A, Montanov 202®, and Coriander oil.

Another embodiment comprises cyclosporin A, Montanov S®, and Coriander oil.

Another embodiment comprises cyclosporin A, Rheozan®, and Coriander oil.

Another embodiment comprises cyclosporin A, Simulsol AS 48®, and Coriander oil.

Another embodiment comprises cyclosporin A, Simulsol SL 4®, and Coriander oil.

Another embodiment comprises cyclosporin A, Simulsol SL 10®, and Coriander oil.

Another embodiment comprises cyclosporin A, Simulsof SL 11W®, and Coriander oil.

Another embodiment comprises cyclosporin A, Simulsol SL 55®, and Coriander oil.

Another embodiment comprises cyclosporin A, Suga Nate 100 and 160®, and Coriander oil.

Another embodiment comprises cyclosporin A, Tego Care 450®, and Coriander oil.

Another embodiment comprises cyclosporin A, Tego Care CG 90®, and Coriander oil.

Another embodiment comprises cyclosporin A, Tego Care PS®, and Coriander oil.

Another embodiment comprises cyclosporin A, Tegosoft PSE 141 G®, and Coriander oil.

Another embodiment comprises cyclosporin A, Tegotens G 826®, and Coriander oil.

Another embodiment comprises cyclosporin A, Triton BG-10 (70%)®, and Coriander oil.

Another embodiment comprises cyclosporin A, Triton CG-110 (60%)®, and Coriander oil.

Another embodiment comprises cyclosporin A, Wickenol 545®, and Coriander oil.

Another embodiment comprises cyclosporin A, an alcohol, and Lavender oil.

Another embodiment comprises cyclosporin A, Diglycerol®, and Lavender oil.

Another embodiment comprises cyclosporin A, Hetoxide GT-80®, and Lavender oil.

Another embodiment comprises cyclosporin A, Lexemul BEO®, and Lavender oil.

Another embodiment comprises cyclosporin A, Polyglycerol-3®, and Lavender oil.

Another embodiment comprises cyclosporin A, Redicote E Series®, and Lavender oil.

Another embodiment comprises cyclosporin A, Simulsol OX 1005L®, and Lavender oil.

Another embodiment comprises cyclosporin A, Stanfax 567®, and Lavender-oil.

Another embodiment comprises cyclosporin A, TA-1618®, and Lavender oil.

Another embodiment comprises cyclosporin A, an amine oxide, and Lavender oil.

Another embodiment comprises cyclosporin A, AO-405®, and Lavender oil.

Another embodiment comprises cyclosporin A, AO-455®, and Lavender oil.

Another embodiment comprises cyclosporin A, AO 728 Special®, and Lavender oil.

Another embodiment comprises cyclosporin A, Barlox 12®, and Lavender oil.

Another embodiment comprises cyclosporin A, Barlox 14®, and Lavender oil.

Another embodiment comprises cyclosporin A, Burcoxide Lo®, and Lavender oil.

Another embodiment comprises cyclosporin A, Caloxamine LO®, and Lavender oil.

Another embodiment comprises cyclosporin A, Chemoxide CAW®, and Lavender oil.

Another embodiment comprises cyclosporin A, Chemoxide LM-30®, and Lavender oil.

Another embodiment comprises cyclosporin A, Chemoxide LO®, and Lavender oil.

Another embodiment comprises cyclosporin A, Chemoxide MO®, and Lavender oil.

Another embodiment comprises cyclosporin A, Colalux CAO-35®, and Lavender oil.

Another embodiment comprises cyclosporin A, Colalux LO®, and Lavender oil.

Another embodiment comprises cyclosporin A, DeMox CAPO®, and Lavender oil.

Another embodiment comprises cyclosporin A, DeMox CSG-30®, and Lavender oil.

Another embodiment comprises cyclosporin A, DeMox LAO®, and Lavender oil.

Another embodiment comprises cyclosporin A, Emcol L®, and Lavender oil.

Another embodiment comprises cyclosporin A, Empigen OB®, and Lavender oil.

Another embodiment comprises cyclosporin A, Empigen OS/A®, and Lavender oil.

Another embodiment comprises cyclosporin A, Foamox CDO®, and Lavender oil.

Another embodiment comprises cyclosporin A, Foamox DMM®, and Lavender oil.

Another embodiment comprises cyclosporin A, Foamox DMS®, and Lavender oil.

Another embodiment comprises cyclosporin A, Genaminox KC®, and Lavender oil.

Another embodiment comprises cyclosporin A, Genaminox LA®, and Lavender oil.

Another embodiment comprises cyclosporin A, Hartofoam SAO®, and Lavender oil.

Another embodiment comprises cyclosporin A, Hartox DMCD®, and Lavender oil.

Another embodiment comprises cyclosporin A, Lipowax DAT®, and Lavender oil.

Another embodiment comprises cyclosporin A, Lipowax PB Pastilles®, and Lavender oil.

Another embodiment comprises cyclosporin A, Mackamine C8®, and Lavender oil.

Another embodiment comprises cyclosporin A, Mackamine C10®, and Lavender oil.

Another embodiment comprises cyclosporin A, Mackamine C14®, and Lavender oil.

Another embodiment comprises cyclosporin A, Mackamine CAO®®, and Lavender oil.

Another embodiment comprises cyclosporin A, Mackamine CO®, and Lavender oil.

Another embodiment comprises cyclosporin A, Mackamine LO®, and Lavender oil.

Another embodiment comprises cyclosporin A, Mackamine O2®, and Lavender oil.

Another embodiment comprises cyclosporin A, Mackamine SAO®, and Lavender oil.

Another embodiment comprises cyclosporin A, Mackamine SO®, and Lavender oil.

Another embodiment comprises cyclosporin A, Mazox KCAO®, and Lavender oil.

Another embodiment comprises cyclosporin A, Monalac MO®, and Lavender oil.

Another embodiment comprises cyclosporin A, Norfox LDA®, and Lavender oil.

Another embodiment comprises cyclosporin A, Rhodamox LO®, and Lavender oil.

Another embodiment comprises cyclosporin A, Scherdamox C-AA®, and Lavender oil.

Another embodiment comprises cyclosporin A, Schercamox DMA®, and Lavender oil.

Another embodiment comprises cyclosporin A, Schercamox DML®, and Lavender oil.

Another embodiment comprises cyclosporin A, Schercamox DMM®, and Lavender oil.

Another embodiment comprises cyclosporin A, Schercamox DMS®, and Lavender oil.

Another embodiment comprises cyclosporin A, Tegotens DO®, and Lavender oil.

Another embodiment comprises cyclosporin A, Tomah AO-14-2®, and Lavender oil.

Another embodiment comprises cyclosporin A, Triaminox CDO®, and Lavender oil.

Another embodiment comprises cyclosporin A, a block polymer, and Lavender oil.

Another embodiment comprises cyclosporin A, AL 2070®, and Lavender oil.

Another embodiment comprises cyclosporin A, Antarox 17-R-2®, and Lavender oil.

Another embodiment comprises cyclosporin A, Antarox 25-R-2®, and Lavender oil.

Another embodiment comprises cyclosporin A, Antarox 31-R-1®, and Lavender oil.

Another embodiment comprises cyclosporin A, Antarox P-84®, and Lavender oil.

Another embodiment comprises cyclosporin A, Antarox P-104/H®, and Lavender oil.

Another embodiment comprises cyclosporin A, Arnox BP-Series®, and Lavender oil.

Another embodiment comprises cyclosporin A, Chemonic 435®, and Lavender oil.

Another embodiment comprises cyclosporin A, Chemonic D-25®, and Lavender oil.

Another embodiment comprises cyclosporin A, Chemonic PL Series®, and Lavender oil.

Another embodiment comprises cyclosporin A, Ethox L-121®, and Lavender oil.

Another embodiment comprises cyclosporin A, Ethox L-122®, and Lavender oil.

Another embodiment comprises cyclosporin A, Genapol PF-10®, and Lavender oil.

Another embodiment comprises cyclosporin A, Genapol PF-20®, and Lavender oil.

Another embodiment comprises cyclosporin A, Genapol PF-40A®, and Lavender oil.

Another embodiment comprises cyclosporin A, Norfox 2-LF®, and Lavender oil.

Another embodiment comprises cyclosporin A, Simulsol NW 342®, and Lavender oil.

Another embodiment comprises cyclosporin A, T-Det BP-1®, and Lavender oil.

Another embodiment comprises cyclosporin A, T-Det XD®, and Lavender oil.

Another embodiment comprises cyclosporin A, T-Det XH®, and Lavender oil.

Another embodiment comprises cyclosporin A, Triton CF-32®, and Lavender oil.

Another embodiment comprises cyclosporin A, Witconol 171®, and Lavender oil.

Another embodiment comprises cyclosporin A, Witconol 324®, and Lavender oil.

Another embodiment comprises cyclosporin A, Witconol 324D®, and Lavender oil.

Another embodiment comprises cyclosporin A, Witconol PD-2000®, and Lavender oil.

Another embodiment comprises cyclosporin A, a Carboxylated Alcohol or Alkylphenol Ethoxylate, and Lavender oil.

Another embodiment comprises cyclosporin A, Emcol CN-6®, and Lavender oil.

Another embodiment comprises cyclosporin A, Ethcarb®, and Lavender oil.

Another embodiment comprises cyclosporin A, Gemtex WNT-Conc®, and Lavender oil.

Another embodiment comprises cyclosporin A, Incrodet TD7-C®, and Lavender oil.

Another embodiment comprises cyclosporin A, Marlinat CM 105/80®, and Lavender oil.

Another embodiment comprises cyclosporin A, Marlowet 1072®, and Lavender oil.

Another embodiment comprises cyclosporin A, Marlowet 4530®, and Lavender oil.

Another embodiment comprises cyclosporin A, Marlowet 4530 LF®, and Lavender oil.

Another embodiment comprises cyclosporin A, Marlowet 4534®, and Lavender oil.

Another embodiment comprises cyclosporin A, Marlowet 4538®, and Lavender oil.

Another embodiment comprises cyclosporin A, Marlowet 4539®, and Lavender oil.

Another embodiment comprises cyclosporin A, Marlowet 4539 LF®, and Lavender oil.

Another embodiment comprises cyclosporin A, Marlowet 4541®, and Lavender oil.

Another embodiment comprises cyclosporin A, Miranate LEC-80®, and Lavender oil.

Another embodiment comprises cyclosporin A, Sandopan B®, and Lavender oil.

Another embodiment comprises cyclosporin A, Sandopan B Modified®, and Lavender oil.

Another embodiment comprises cyclosporin A, Sandopan LS-24 Gel®, and Lavender oil.

Another embodiment comprises cyclosporin A, Surfine T-A®, and Lavender oil.

Another embodiment comprises cyclosporin A, a Carboxylic Acid/Fatty Acid, and Lavender oil.

Another embodiment comprises cyclosporin A, Colaterge RAM®, and Lavender oil.

Another embodiment comprises cyclosporin A, Colatrope INC®, and Lavender oil.

Another embodiment comprises cyclosporin A, Crodacid B®, and Lavender oil.

Another embodiment comprises cyclosporin A, DeTrope CA-100®, and Lavender oil.

Another embodiment comprises cyclosporin A, Latol MTO®, and Lavender oil.

Another embodiment comprises cyclosporin A, Lumulse CC-33 K®, and Lavender oil.

Another embodiment comprises cyclosporin A, Mulls 2218®, and Lavender oil.

Another embodiment comprises cyclosporin A, OL-600®, and Lavender oil.

Another embodiment comprises cyclosporin A, OL-800®, and Lavender oil.

Another embodiment comprises cyclosporin A, R-910®, and Lavender oil.

Another embodiment comprises cyclosporin A, S-210®, and Lavender oil.

Another embodiment comprises cyclosporin A, Sandopan DTC Acid®, and Lavender oil.

Another embodiment comprises cyclosporin A, Sandopan LS 24 N®, and Lavender oil.

Another embodiment comprises cyclosporin A, Sandopan MA-18®, and Lavender oil.

Another embodiment comprises cyclosporin A, an Ethoxylated Alcohol, and Lavender oil.

Another embodiment comprises cyclosporin A, Adsee 799®, and Lavender oil.

Another embodiment comprises cyclosporin A, Adsee 799®, and Lavender oil.

Another embodiment comprises cyclosporin A, Alfonic 610-3.5®, and Lavender oil.

Another embodiment comprises cyclosporin A, Alfonic 810-2®, and Lavender oil.

Another embodiment comprises cyclosporin A, Alfonic 810-6®, and Lavender oil.

Another embodiment comprises cyclosporin A, Alfonic 1012-30, and Lavender oil.

Another embodiment comprises cyclosporin A, Alfonic 1012-5®, and Lavender oil.

Another embodiment comprises cyclosporin A, Alfonic 1216CO-1.5®, and Lavender oil.

Another embodiment comprises cyclosporin A, Alfonic 121 6CO-7®, and Lavender oil.

Another embodiment comprises cyclosporin A, Alfonic 1412-3®, and Lavender oil.

Another embodiment comprises cyclosporin A, Alfonic 1412-7®, and Lavender oil.

Another embodiment comprises cyclosporin A, Arlasolve 200®, and Lavender oil.

Another embodiment comprises cyclosporin A, Arlasolve 200 Liquid®, and Lavender oil.

Another embodiment comprises cyclosporin A, Armix 180-C®, and Lavender oil.

Another embodiment comprises cyclosporin A, Armix 183®, and Lavender oil.

Another embodiment comprises cyclosporin A, Armul 2404®, and Lavender oil.

Another embodiment comprises cyclosporin A, Armul 2404®, and Lavender oil.

Another embodiment comprises cyclosporin A, Atlas EMJ-C®, and Lavender oil.

Another embodiment comprises cyclosporin A, Atlas G-2109®, and Lavender oil.

Another embodiment comprises cyclosporin A, Atlas G-3886®, and Lavender oil.

Another embodiment comprises cyclosporin A, Atlas G-3890®, and Lavender oil.

Another embodiment comprises cyclosporin A, Bio Soft E-200®, and Lavender oil.

Another embodiment comprises cyclosporin A, Bio Soft E-300®, and Lavender oil.

Another embodiment comprises cyclosporin A, Bio Soft E-400®, and Lavender oil.

Another embodiment comprises cyclosporin A, Bio Soft EN 600®, and Lavender oil.

Another embodiment comprises cyclosporin A, Bio Soft TD-400®, and Lavender oil.

Another embodiment comprises cyclosporin A, Bio Soft TD-630®, and Lavender oil.

Another embodiment comprises cyclosporin A, Brij 30®, and Lavender oil.

Another embodiment comprises cyclosporin A, Brij 52®, and Lavender oil.

Another embodiment comprises cyclosporin A, Brij 56®, and Lavender oil.

Another embodiment comprises cyclosporin A, Brij 58®, and Lavender oil.

Another embodiment comprises cyclosporin A, Brij 72®, and Lavender oil.

Another embodiment comprises cyclosporin A, Brij 76®, and Lavender oil.

Another embodiment comprises cyclosporin A, Brij 78®, and Lavender oil.

Another embodiment comprises cyclosporin A, Brij 93®, and Lavender oil.

Another embodiment comprises cyclosporin A, Brij 97®, and Lavender oil.

Another embodiment comprises cyclosporin A, Brij 98®, and Lavender oil.

Another embodiment comprises cyclosporin A, Brij 700®, and Lavender oil.

Another embodiment comprises cyclosporin A, Brij 700 S®, and Lavender oil.

Another embodiment comprises cyclosporin A, Brij 721®, and Lavender oil.

Another embodiment comprises cyclosporin A, Brij 721 S®, and Lavender oil.

Another embodiment comprises cyclosporin A, Burcoterge CDG®, and Lavender oil.

Another embodiment comprises cyclosporin A, Canasol AT 600®, and Lavender oil.

Another embodiment comprises cyclosporin A, Canasol AT 800®, and Lavender oil.

Another embodiment comprises cyclosporin A, Canasol AT 1200®, and Lavender oil.

Another embodiment comprises cyclosporin A, Canasol BJ 35®, and Lavender oil.

Another embodiment comprises cyclosporin A, Canasol BJ 36®, and Lavender oil.

Another embodiment comprises cyclosporin A, Canasol BJ 52®, and Lavender oil.

Another embodiment comprises cyclosporin A, Canasol BJ 58®, and Lavender oil.

Another embodiment comprises cyclosporin A, Canasol BJ 72®, and Lavender oil.

Another embodiment comprises cyclosporin A, Canasol BJ 78®, and Lavender oil.

Another embodiment comprises cyclosporin A, Canasol BJ 98®, and Lavender oil.

Another embodiment comprises cyclosporin A, Canasol BJ 307®, and Lavender oil.

Another embodiment comprises cyclosporin A, Cerfak 1400®, and Lavender oil.

Another embodiment comprises cyclosporin A, Cetomacrogol 1000 BP®, and Lavender oil.

Another embodiment comprises cyclosporin A, Chemonic C-2®, and Lavender oil.

Another embodiment comprises cyclosporin A, Chemonic C-10®, and Lavender oil.

Another embodiment comprises cyclosporin A, Chemonic C-20®, and Lavender oil.

Another embodiment comprises cyclosporin A, Chemonic CT-12®, and Lavender oil.

Another embodiment comprises cyclosporin A, Chemonic CT-20®, and Lavender oil.

Another embodiment comprises cyclosporin A, Chemonic CT-30®, and Lavender oil.

Another embodiment comprises cyclosporin A, Chemonic CT-55®, and Lavender oil.

Another embodiment comprises cyclosporin A, Chemonic G-7®, and Lavender oil.

Another embodiment comprises cyclosporin A, Chemonic G-26®, and Lavender oil.

Another embodiment comprises cyclosporin A, Chemonic L-4®, and Lavender oil.

Another embodiment comprises cyclosporin A, Chemonic L-7®, and Lavender oil.

Another embodiment comprises cyclosporin A, Chemonic L-12®, and Lavender oil.

Another embodiment comprises cyclosporin A, Chemonic L-23®, and Lavender oil.

Another embodiment comprises cyclosporin A, Chemonic O-2®, and Lavender oil.

Another embodiment comprises cyclosporin A, Chemonic O-5®, and Lavender oil.

Another embodiment comprises cyclosporin A, Chemonic O-10®, and Lavender oil.

Another embodiment comprises cyclosporin A, Chemonic O-20®, and Lavender oil.

Another embodiment comprises cyclosporin A, Chemonic S-2®, and Lavender oil.

Another embodiment comprises cyclosporin A, Chemonic S-10®, and Lavender oil.

Another embodiment comprises cyclosporin A, Chemonic S-20®, and Lavender oil.

Another embodiment comprises cyclosporin A, Colamulse FE®, and Lavender oil.

Another embodiment comprises cyclosporin A, Cremophor A 20®, and Lavender oil.

Another embodiment comprises cyclosporin A, Cremophor SA 2®, and Lavender oil.

Another embodiment comprises cyclosporin A, Dehydol 100®, and Lavender oil.

Another embodiment comprises cyclosporin A, Dehydol O-4®, and Lavender oil.

Another embodiment comprises cyclosporin A, Delonic C-18®, and Lavender oil.

Another embodiment comprises cyclosporin A, DeSonic 6T®, and Lavender oil.

Another embodiment comprises cyclosporin A, DeSonic 9D®, and Lavender oil.

Another embodiment comprises cyclosporin A, DeSonic 9T®, and Lavender oil.

Another embodiment comprises cyclosporin A, DeSonic 12D®, and Lavender oil.

Another embodiment comprises cyclosporin A, DeSonic 12T®, and Lavender oil.

Another embodiment comprises cyclosporin A, DeSonic 15T®, and Lavender oil.

Another embodiment comprises cyclosporin A, DeSonic TDA-9®, and Lavender oil.

Another embodiment comprises cyclosporin A, DeThox GLG-7®, and Lavender oil.

Another embodiment comprises cyclosporin A, DeThox GLG-26®, and Lavender oil.

Another embodiment comprises cyclosporin A, DeThox LA-4®, and Lavender oil.

Another embodiment comprises cyclosporin A, DeThox LA-23®, and Lavender oil.

Another embodiment comprises cyclosporin A, DeThox SA-80®, and Lavender oil.

Another embodiment comprises cyclosporin A, Disponil O5®, and Lavender oil.

Another embodiment comprises cyclosporin A, Eccoterge EO-41B®, and Lavender oil.

Another embodiment comprises cyclosporin A, Empilan KA2.5/90®, and Lavender oil.

Another embodiment comprises cyclosporin A, Empilan KA5/90®, and Lavender oil.

Another embodiment comprises cyclosporin A, Empilan KM-20®, and Lavender oil.

Another embodiment comprises cyclosporin A, Empilan KM-50®, and Lavender oil.

Another embodiment comprises cyclosporin A, Empilan L-23®, and Lavender oil.

Another embodiment comprises cyclosporin A, Ethylan 25-3®, and Lavender oil.

Another embodiment comprises cyclosporin A, Ethylan 1204®, and Lavender oil.

Another embodiment comprises cyclosporin A, Ethylan DA-4®, and Lavender oil.

Another embodiment comprises cyclosporin A, Ethylan LA-230®, and Lavender oil.

Another embodiment comprises cyclosporin A, Ethylan SN®, and Lavender oil.

Another embodiment comprises cyclosporin A, Ethylan TD-60®, and Lavender oil.

Another embodiment comprises cyclosporin A, Ethylan TD-100®, and Lavender oil.

Another embodiment comprises cyclosporin A, Ethylan TD-1407®, and Lavender oil.

Another embodiment comprises cyclosporin A, Eumulgin B1®, and Lavender oil.

Another embodiment comprises cyclosporin A, Eumulgin B2®, and Lavender oil.

Another embodiment comprises cyclosporin A, Eumulgin B3®, and Lavender oil.

Another embodiment comprises cyclosporin A, Eumulgin O-10®, and Lavender oil.

Another embodiment comprises cyclosporin A, Flo Mo 80/20®, and Lavender oil.

Another embodiment comprises cyclosporin A, Flo Mo Low Foam®, and Lavender oil.

Another embodiment comprises cyclosporin A, Forlan C-24®, and Lavender oil.

Another embodiment comprises cyclosporin A, Genapol 1454®, and Lavender oil.

Another embodiment comprises cyclosporin A, Genapol BA-020®, and Lavender oil.

Another embodiment comprises cyclosporin A, Genapol BA-040®, and Lavender oil.

Another embodiment comprises cyclosporin A, Genapol C-100®, and Lavender oil.

Another embodiment comprises cyclosporin A, Genapol DA 060®, and Lavender oil.

Another embodiment comprises cyclosporin A, Genapol HS 020®, and Lavender oil.

Another embodiment comprises cyclosporin A, Genapol HS 200®, and Lavender oil.

Another embodiment comprises cyclosporin A, Genapol ID-040®, and Lavender oil.

Another embodiment comprises cyclosporin A, Genapol ID-060®, and Lavender oil.

Another embodiment comprises cyclosporin A, Genapol ID-090®, and Lavender oil.

Another embodiment comprises cyclosporin A, Genapol LA 010®, and Lavender oil.

Another embodiment comprises cyclosporin A, Genapol LA 020®, and Lavender oil.

Another embodiment comprises cyclosporin A, Genapol LA 030®, and Lavender oil.

Another embodiment comprises cyclosporin A, Genapol LA 040®, and Lavender oil.

Another embodiment comprises cyclosporin A, Genapol LA 050®, and Lavender oil.

Another embodiment comprises cyclosporin A, Genapol LA 060®, and Lavender oil.

Another embodiment comprises cyclosporin A, Genapol LA 070®, and Lavender oil.

Another embodiment comprises cyclosporin A, Genapol LA 070S®, and Lavender oil.

Another embodiment comprises cyclosporin A, Genapol LA 230®, and Lavender oil.

Another embodiment comprises cyclosporin A, Genapol O 020®, and Lavender oil.

Another embodiment comprises cyclosporin A, Genapol O 050®, and Lavender oil.

Another embodiment comprises cyclosporin A, Genapol O 100®, and Lavender oil.

Another embodiment comprises cyclosporin A, Genapol O 200®, and Lavender oil.

Another embodiment comprises cyclosporin A, Genapol SA 030®, and Lavender oil.

Another embodiment comprises cyclosporin A, Genapol SA 120®, and Lavender oil.

Another embodiment comprises cyclosporin A, Genapol T-020®, and Lavender oil.

Another embodiment comprises cyclosporin A, Genapol UD-030®, and Lavender oil.

Another embodiment comprises cyclosporin A, Genapol UD-050®, and Lavender oil.

Another embodiment comprises cyclosporin A, Genapol UD-070®, and Lavender oil.

Another embodiment comprises cyclosporin A, Genapol UD-079®, and Lavender oil.

Another embodiment comprises cyclosporin A, Genapol UD-080®, and Lavender oil.

Another embodiment comprises cyclosporin A, Genapol UD-110®, and Lavender oil.

Another embodiment comprises cyclosporin A, Genapol X 030®, and Lavender oil.

Another embodiment comprises cyclosporin A, Genapol X 050®, and Lavender oil.

Another embodiment comprises cyclosporin A, Genapol X 060®, and Lavender oil.

Another embodiment comprises cyclosporin A, Genapol X 070®, and Lavender oil.

Another embodiment comprises cyclosporin A, Genapol X 080®, and Lavender oil.

Another embodiment comprises cyclosporin A, Genapol X 100®, and Lavender oil.

Another embodiment comprises cyclosporin A, Genapol X159®, and Lavender oil.

Another embodiment comprises cyclosporin A, Generol 122 E5®, and Lavender oil.

Another embodiment comprises cyclosporin A, Generol 122 E25®, and Lavender oil.

Another embodiment comprises cyclosporin A, Hostacerin T-3®, and Lavender oil.

Another embodiment comprises cyclosporin A, Iconol LF 110®, and Lavender oil.

Another embodiment comprises cyclosporin A, Incropol CS-20®, and Lavender oil.

Another embodiment comprises cyclosporin A, Lexemul CS-20®, and Lavender oil.

Another embodiment comprises cyclosporin A, Liponic EG-1®, and Lavender oil.

Another embodiment comprises cyclosporin A, Lipowax D®, and Lavender oil.

Another embodiment comprises cyclosporin A, Lipowax G®, and Lavender oil.

Another embodiment comprises cyclosporin A, Lipowax NI®, and Lavender oil.

Another embodiment comprises cyclosporin A, Lipowax P®, and Lavender oil.

Another embodiment comprises cyclosporin A, Lipowax P-31®, and Lavender oil.

Another embodiment comprises cyclosporin A, Lipowax PR®, and Lavender oil.

Another embodiment comprises cyclosporin A, Lumulse CS-20®, and Lavender oil.

Another embodiment comprises cyclosporin A, Macol CSA-2®, and Lavender oil.

Another embodiment comprises cyclosporin A, Marlox B 24/50®, and Lavender oil.

Another embodiment comprises cyclosporin A, Mazawet 77®, and Lavender oil.

Another embodiment comprises cyclosporin A, Norfox 1713®, and Lavender oil.

Another embodiment comprises cyclosporin A, Norfox 2579®, and Lavender oil.

Another embodiment comprises cyclosporin A, Norfox Lo Foam®, and Lavender oil.

Another embodiment comprises cyclosporin A, Promulgen D®, and Lavender oil.

Another embodiment comprises cyclosporin A, Promulgen G®, and Lavender oil.

Another embodiment comprises cyclosporin A, Renex 30®, and Lavender oil.

Another embodiment comprises cyclosporin A, Renex 36®, and Lavender oil.

Another embodiment comprises cyclosporin A, Rhodasurf A 24®, and Lavender oil.

Another embodiment comprises cyclosporin A, Rhodasurf AAE-10®, and Lavender oil.

Another embodiment comprises cyclosporin A, Rhodasurf BEH-25®, and Lavender oil.

Another embodiment comprises cyclosporin A, Rhodasurf BEH-40®, and Lavender oil.

Another embodiment comprises cyclosporin A, Rhodasurf DA 530®, and Lavender oil.

Another embodiment comprises cyclosporin A, Rhodasurf DA 630®, and Lavender oil.

Another embodiment comprises cyclosporin A, Rhodasurf DA 639®, and Lavender oil.

Another embodiment comprises cyclosporin A, Rhodasurf LAN-23®, and Lavender oil.

Another embodiment comprises cyclosporin A, Rhodasurf ON-870®, and Lavender oil.

Another embodiment comprises cyclosporin A, Rhodasurf ON-877®, and Lavender oil.

Another embodiment comprises cyclosporin A, Rhodasurf TB-970 FLK®, and Lavender oil.

Another embodiment comprises cyclosporin A, Ritacet-20®, and Lavender oil.

Another embodiment comprises cyclosporin A, Ritachol 1000®, and Lavender oil.

Another embodiment comprises cyclosporin A, Ritachol 2000®, and Lavender oil.

Another embodiment comprises cyclosporin A, Ritachol 5000®, and Lavender oil.

Another embodiment comprises cyclosporin A, Ritox 35®, and Lavender oil.

Another embodiment comprises cyclosporin A, Surfonic DA-4®, and Lavender oil.

Another embodiment comprises cyclosporin A, Surfonic DA-6®, and Lavender oil.

Another embodiment comprises cyclosporin A, Surfonic L46-7®, and Lavender oil.

Another embodiment comprises cyclosporin A, Surfonic POA®, and Lavender oil.

Another embodiment comprises cyclosporin A, Synthrapol KB®, and Lavender oil.

Another embodiment comprises cyclosporin A, Teginacid®, and Lavender oil.

Another embodiment comprises cyclosporin A, Teginacid C®, and Lavender oil.

Another embodiment comprises cyclosporin A, Tegotens EC 11®, and Lavender oil.

Another embodiment comprises cyclosporin A, Tinegal NA®, and Lavender oil.

Another embodiment comprises cyclosporin A, Tomadol 400®, and Lavender oil.

Another embodiment comprises cyclosporin A, Tomadol 600®, and Lavender oil.

Another embodiment comprises cyclosporin A, Tomadol 900®, and Lavender oil.

Another embodiment comprises cyclosporin A, Uniperol O®, and Lavender oil.

Another embodiment comprises cyclosporin A, Witconol SN Series®, and Lavender oil.

Another embodiment comprises cyclosporin A, an Ethoxylated Alkylphenol, and Lavender oil.

Another embodiment comprises cyclosporin A, Antarox LF-222®, and Lavender oil.

Another embodiment comprises cyclosporin A, Atlox 775®, and Lavender oil.

Another embodiment comprises cyclosporin A, Caloxylate N-9®, and Lavender oil.

Another embodiment comprises cyclosporin A, Canasol NF-1000®, and Lavender oil.

Another embodiment comprises cyclosporin A, Canasol NF-3000®, and Lavender oil.

Another embodiment comprises cyclosporin A, Canasol NF-3070®, and Lavender oil.

Another embodiment comprises cyclosporin A, Canasol OF 1670®, and Lavender oil.

Another embodiment comprises cyclosporin A, Canasol OF 2570®, and Lavender oil.

Another embodiment comprises cyclosporin A, Canasol OF 4070®, and Lavender oil.

Another embodiment comprises cyclosporin A, Chemax DNP-8®, and Lavender oil.

Another embodiment comprises cyclosporin A, Chemax DNP-18®, and Lavender oil.

Another embodiment comprises cyclosporin A, Chemax DNP-150/50®, and Lavender oil.

Another embodiment comprises cyclosporin A, DeSonic 1.5N®, and Lavender oil.

Another embodiment comprises cyclosporin A, DeSonic 4N®, and Lavender oil.

Another embodiment comprises cyclosporin A, DeSonic 5N®, and Lavender oil.

Another embodiment comprises cyclosporin A, DeSonic 6D®, and Lavender oil.

Another embodiment comprises cyclosporin A, DeSonic 6N®, and Lavender oil.

Another embodiment comprises cyclosporin A, DeSonic 7N®, and Lavender oil.

Another embodiment comprises cyclosporin A, DeSonic 9N®, and Lavender oil.

Another embodiment comprises cyclosporin A, DeSonic 10D®, and Lavender oil.

Another embodiment comprises cyclosporin A, DeSonic 11N®, and Lavender oil.

Another embodiment comprises cyclosporin A, DeSonic 12N®, and Lavender oil.

Another embodiment comprises cyclosporin A, DeSonic 15N®, and Lavender oil.

Another embodiment comprises cyclosporin A, DeSonic 20N®, and Lavender oil.

Another embodiment comprises cyclosporin A, Eccoscour RC®, and Lavender oil.

Another embodiment comprises cyclosporin A, Eccoterge EO-100®, and Lavender oil.

Another embodiment comprises cyclosporin A, Emulsifier 632/90%®, and Lavender oil.

Another embodiment comprises cyclosporin A, Geronol AG-821®, and Lavender oil.

Another embodiment comprises cyclosporin A, Gradonic N-95®, and Lavender oil.

Another embodiment comprises cyclosporin A, Hetoxide NP-4®, and Lavender oil.

Another embodiment comprises cyclosporin A, Hetoxide NP-30®, and Lavender oil.

Another embodiment comprises cyclosporin A, Hostapal N-100®, and Lavender oil.

Another embodiment comprises cyclosporin A, Hostapal N-110®, and Lavender oil.

Another embodiment comprises cyclosporin A, Igepal CTA-639W®, and Lavender oil.

Another embodiment comprises cyclosporin A, Igepal DAP-9®, and Lavender oil.

Another embodiment comprises cyclosporin A, Igepal OD-410®, and Lavender oil.

Another embodiment comprises cyclosporin A, Igepal SS-837®, and Lavender oil.

Another embodiment comprises cyclosporin A, Lipocol NP-9 USP®, and Lavender oil.

Another embodiment comprises cyclosporin A, Macol DNP-10®, and Lavender oil.

Another embodiment comprises cyclosporin A, Marlophen NP 5®, and Lavender oil.

Another embodiment comprises cyclosporin A, Marlophen P 1®, and Lavender oil.

Another embodiment comprises cyclosporin A, Surfonic NB®, and Lavender oil.

Another embodiment comprises cyclosporin A, Surfonic OPB-307®, and Lavender oil.

Another embodiment comprises cyclosporin A, Surfonic OPB-407®, and Lavender oil.

Another embodiment comprises cyclosporin A, Syn Fac 334®, and Lavender oil.

Another embodiment comprises cyclosporin A, Syn Fac 8216®, and Lavender oil.

Another embodiment comprises cyclosporin A, Triton N-57®, and Lavender oil.

Another embodiment comprises cyclosporin A, Trycol 6956®, and Lavender oil.

Another embodiment comprises cyclosporin A, Trycol 6961®, and Lavender oil.

Another embodiment comprises cyclosporin A, Trycol 6964®, and Lavender oil.

Another embodiment comprises cyclosporin A, Trycol 6969®, and Lavender oil.

Another embodiment comprises cyclosporin A, Trycol 6974®, and Lavender oil.

Another embodiment comprises cyclosporin A, Witbreak DRB-127®, and Lavender oil.

Another embodiment comprises cyclosporin A, Witbreak DRB-127®, and Lavender oil.

Another embodiment comprises cyclosporin A, Witconol NP Series®, and Lavender oil.

Another embodiment comprises cyclosporin A, an Ethoxylated Aryl Phenol, and Lavender oil.

Another embodiment comprises cyclosporin A, Soprophor BSU®, and Lavender oil.

Another embodiment comprises cyclosporin A, Soprophor CY/8®, and Lavender oil.

Another embodiment comprises cyclosporin A, Soprophor S/25®, and Lavender oil.

Another embodiment comprises cyclosporin A, Witconol NIO®, and Lavender oil.

Another embodiment comprises cyclosporin A, Witconol NIW®, and Lavender oil.

Another embodiment comprises cyclosporin A, Witconol S-100®, and Lavender oil.

Another embodiment comprises cyclosporin A, an Ethoxylated Fatty Acid, and Lavender oil.

Another embodiment comprises cyclosporin A, Aldo PGHMS®, and Lavender oil.

Another embodiment comprises cyclosporin A, Alkamuls TO-15/HR®, and Lavender oil.

Another embodiment comprises cyclosporin A, Armotan AL-69-66®, and Lavender oil.

Another embodiment comprises cyclosporin A, Cerasynt 840®, and Lavender oil.

Another embodiment comprises cyclosporin A, Cerasynt 945®, and Lavender oil.

Another embodiment comprises cyclosporin A, Crystal Inhibitor No. 5®, and Lavender oil.

Another embodiment comprises cyclosporin A, DeThox Acid L-9®, and Lavender oil.

Another embodiment comprises cyclosporin A, DeThox Acid S-8®, and Lavender oil.

Another embodiment comprises cyclosporin A, Ethofat 242/25®, and Lavender oil.

Another embodiment comprises cyclosporin A, Hydropalat 65®, and Lavender oil.

Another embodiment comprises cyclosporin A, Lipo EGMS®, and Lavender oil.

Another embodiment comprises cyclosporin A, Lipopeg 2 DL®, and Lavender oil.

Another embodiment comprises cyclosporin A, Lipopeg 4 DL®, and Lavender oil.

Another embodiment comprises cyclosporin A, Lipopeg 4-L®, and Lavender oil.

Another embodiment comprises cyclosporin A, Lipopeg 39-S®, and Lavender oil.

Another embodiment comprises cyclosporin A, Lipopeg 4-S®, and Lavender oil.

Another embodiment comprises cyclosporin A, Lipopeg 10-S®, and Lavender oil.

Another embodiment comprises cyclosporin A, Lipopeg 100-S®, and Lavender oil.

Another embodiment comprises cyclosporin A, Lipopeg 6000 DS®, and Lavender oil.

Another embodiment comprises cyclosporin A, Lumulse 40-L®, and Lavender oil.

Another embodiment comprises cyclosporin A, Lumulse 40-S®, and Lavender oil.

Another embodiment comprises cyclosporin A, Lumulse 42-L®, and Lavender oil.

Another embodiment comprises cyclosporin A, Lumulse 42-S®, and Lavender oil.

Another embodiment comprises cyclosporin A, Lumulse 100-S®, and Lavender oil.

Another embodiment comprises cyclosporin A, Lumulse 602-S®, and Lavender oil.

Another embodiment comprises cyclosporin A, Magrabar PGE-20-0®, and Lavender oil.

Another embodiment comprises cyclosporin A, Magrabar PGE-20L®, and Lavender oil.

Another embodiment comprises cyclosporin A, Magrabar PGE-20T®, and Lavender oil.

Another embodiment comprises cyclosporin A, Magrabar PGE-22-0®, and Lavender oil.

Another embodiment comprises cyclosporin A, Magrabar PGE-22L®, and Lavender oil.

Another embodiment comprises cyclosporin A, Magrabar PGE-22T®, and Lavender oil.

Another embodiment comprises cyclosporin A, Magrabar PGE-40-0®, and Lavender oil.

Another embodiment comprises cyclosporin A, Magrabar PGE-40L®, and Lavender oil.

Another embodiment comprises cyclosporin A, Magrabar PGE-40T®, and Lavender oil.

Another embodiment comprises cyclosporin A, Magrabar PGE-42-0®, and Lavender oil.

Another embodiment comprises cyclosporin A, Magrabar PGE-42L®, and Lavender oil.

Another embodiment comprises cyclosporin A, Magrabar PGE-42T®, and Lavender oil.

Another embodiment comprises cyclosporin A, Magrabar PGE-60-0®, and Lavender oil.

Another embodiment comprises cyclosporin A, Magrabar PGE-60L®, and Lavender oil.

Another embodiment comprises cyclosporin A, Magrabar PGE-60T®, and Lavender oil.

Another embodiment comprises cyclosporin A, Magrabar PGE-62-0®, and Lavender oil.

Another embodiment comprises cyclosporin A, Magrabar PGE-62L®, and Lavender oil.

Another embodiment comprises cyclosporin A, Magrabar PGE-62T®, and Lavender oil.

Another embodiment comprises cyclosporin A, Mapeg S-40K®, and Lavender oil.

Another embodiment comprises cyclosporin A, Marlowet OTS®, and Lavender oil.

Another embodiment comprises cyclosporin A, Naturechem PGR®, and Lavender oil.

Another embodiment comprises cyclosporin A, PG No. 4®, and Lavender oil.

Another embodiment comprises cyclosporin A, Renex 20®, and Lavender oil.

Another embodiment comprises cyclosporin A, Ritox 52®, and Lavender oil.

Another embodiment comprises cyclosporin A, Ritox 53®, and Lavender oil.

Another embodiment comprises cyclosporin A, Ritox 59®, and Lavender oil.

Another embodiment comprises cyclosporin A, Surfax 8916/A®, and Lavender oil.

Another embodiment comprises cyclosporin A, Tego Acid S 40 P®, and Lavender oil.

Another embodiment comprises cyclosporin A, Tego Acid S 100 P®, and Lavender oil.

Another embodiment comprises cyclosporin A, Tween 20®, and Lavender oil.

Another embodiment comprises cyclosporin A, Volpo 131®, and Lavender oil.

Another embodiment comprises cyclosporin A, an Ethoxylated Fatty Ester or Oil, and Lavender oil.

Another embodiment comprises cyclosporin A, Acconon 6-C10®, and Lavender oil.

Another embodiment comprises cyclosporin A, Acconon CC-6®, and Lavender oil.

Another embodiment comprises cyclosporin A, Acconon CO-7®, and Lavender oil.

Another embodiment comprises cyclosporin A, Aldosperse 40/60 FG®, and Lavender oil.

Another embodiment comprises cyclosporin A, Aldosperse ML-23®, and Lavender oil.

Another embodiment comprises cyclosporin A, Aldosperse MS-20 FG®, and Lavender oil.

Another embodiment comprises cyclosporin A, Alkamuls EL-620®, and Lavender oil.

Another embodiment comprises cyclosporin A, Alkamuls EL-719®, and Lavender oil.

Another embodiment comprises cyclosporin A, Alkamuls EL-985®, and Lavender oil.

Another embodiment comprises cyclosporin A, Arlatone G®, and Lavender oil.

Another embodiment comprises cyclosporin A, Arlatone T®, and Lavender oil.

Another embodiment comprises cyclosporin A, Atlas G-1045A®, and Lavender oil.

Another embodiment comprises cyclosporin A, Atlas G-1086®, and Lavender oil.

Another embodiment comprises cyclosporin A, Atlas G-1087®, and Lavender oil.

Another embodiment comprises cyclosporin A, Atlas G-1089®, and Lavender oil.

Another embodiment comprises cyclosporin A, Atlas G-1096®, and Lavender oil.

Another embodiment comprises cyclosporin A, Atlas G-1292®, and Lavender oil.

Another embodiment comprises cyclosporin A, Atlas G-1293®, and Lavender oil.

Another embodiment comprises cyclosporin A, Atlas G-1300®, and Lavender oil.

Another embodiment comprises cyclosporin A, Atlas G-7076®, and Lavender oil.

Another embodiment comprises cyclosporin A, Capmul EMG®, and Lavender oil.

Another embodiment comprises cyclosporin A, Chemonic CO-40®, and Lavender oil.

Another embodiment comprises cyclosporin A, Chemonic LI-3®, and Lavender oil.

Another embodiment comprises cyclosporin A, Chemonic LI-7®, and Lavender oil.

Another embodiment comprises cyclosporin A, Cirrasol GM®, and Lavender oil.

Another embodiment comprises cyclosporin A, Cremophor CO 40®, and Lavender oil.

Another embodiment comprises cyclosporin A, Cremophor CO 410®, and Lavender oil.

Another embodiment comprises cyclosporin A, Cremophor EL®, and Lavender oil.

Another embodiment comprises cyclosporin A, Cremophor GC7®, and Lavender oil.

Another embodiment comprises cyclosporin A, Cremophor RH-40®, and Lavender oil.

Another embodiment comprises cyclosporin A, Crovol A-40®, and Lavender oil.

Another embodiment comprises cyclosporin A, Crovol A-70®, and Lavender oil.

Another embodiment comprises cyclosporin A, Crovol M-70®, and Lavender oil.

Another embodiment comprises cyclosporin A, Crovol PK-70®, and Lavender oil.

Another embodiment comprises cyclosporin A, Cutina E-24®, and Lavender oil.

Another embodiment comprises cyclosporin A, Dacospin 12-R®, and Lavender oil.

Another embodiment comprises cyclosporin A, Dehymuls HRE-7®, and Lavender oil.

Another embodiment comprises cyclosporin A, DeSonic 30C®, and Lavender oil.

Another embodiment comprises cyclosporin A, DeSonic 36C®, and Lavender oil.

Another embodiment comprises cyclosporin A, DeSonic 40C®, and Lavender oil.

Another embodiment comprises cyclosporin A, Durfax 60®, and Lavender oil.

Another embodiment comprises cyclosporin A, Durfax 65®, and Lavender oil.

Another embodiment comprises cyclosporin A, Durfax 80®, and Lavender oil.

Another embodiment comprises cyclosporin A, Durfax EOM®, and Lavender oil.

Another embodiment comprises cyclosporin A, Eccoterge NF-2®, and Lavender oil.

Another embodiment comprises cyclosporin A, Emulpon CO-360®, and Lavender oil.

Another embodiment comprises cyclosporin A, Emulpon CO-550®, and Lavender oil.

Another embodiment comprises cyclosporin A, Emulsogen EL®, and Lavender oil.

Another embodiment comprises cyclosporin A, Emulsogen HCO 040®, and Lavender oil.

Another embodiment comprises cyclosporin A, Emulsogen HCO 060®, and Lavender oil.

Another embodiment comprises cyclosporin A, Emulsynt 1055®, and Lavender oil.

Another embodiment comprises cyclosporin A, Ethox 3095®, and Lavender oil.

Another embodiment comprises cyclosporin A, Eumulgin RO-40®, and Lavender oil.

Another embodiment comprises cyclosporin A, Genapol G-260®, and Lavender oil.

Another embodiment comprises cyclosporin A, Glycosperse L-20®, and Lavender oil.

Another embodiment comprises cyclosporin A, Glycosperse O-5®, and Lavender oil.

Another embodiment comprises cyclosporin A, Glycosperse O-20a, and Lavender oil.

Another embodiment comprises cyclosporin A, Glycosperse O-20 FG®, and Lavender oil.

Another embodiment comprises cyclosporin A, Glycosperse S-20®, and Lavender oil.

Another embodiment comprises cyclosporin A, Glycosperse S-20 FG®, and Lavender oil.

Another embodiment comprises cyclosporin A, Glycosperse TS-20®, and Lavender oil.

Another embodiment comprises cyclosporin A, Glycosperse TS-20 FG®, and Lavender oil.

Another embodiment comprises cyclosporin A, Hetan SL®, and Lavender oil.

Another embodiment comprises cyclosporin A, Hetan SO®, and Lavender oil.

Another embodiment comprises cyclosporin A, Hetan SS®, and Lavender oil.

Another embodiment comprises cyclosporin A, Hetoxide C-2®, and Lavender oil.

Another embodiment comprises cyclosporin A, Hetoxide C-9®, and Lavender oil.

Another embodiment comprises cyclosporin A, Hetoxide C-15®, and Lavender oil.

Another embodiment comprises cyclosporin A, Hetoxide C-25®, and Lavender oil.

Another embodiment comprises cyclosporin A, Hetoxide C-40®, and Lavender oil.

Another embodiment comprises cyclosporin A, Hetoxide C-200®, and Lavender oil.

Another embodiment comprises cyclosporin A, Hetoxide C-200-50%®, and Lavender oil.

Another embodiment comprises cyclosporin A, Hetoxide GC-30®, and Lavender oil.

Another embodiment comprises cyclosporin A, Hetoxide HC-60®, and Lavender oil.

Another embodiment comprises cyclosporin A, Ice No. 2®, and Lavender oil.

Another embodiment comprises cyclosporin A, Incrocas 30/40®, and Lavender oil.

Another embodiment comprises cyclosporin A, Lexol EC®, and Lavender oil.

Another embodiment comprises cyclosporin A, Lexol EO®, and Lavender oil.

Another embodiment comprises cyclosporin A, Lipocol HCO-40®, and Lavender oil.

Another embodiment comprises cyclosporin A, Lipocol HCO-60®, and Lavender oil.

Another embodiment comprises cyclosporin A, Lipocol O-3 Special®, and Lavender oil.

Another embodiment comprises cyclosporin A, Lipopeg 2-L®, and Lavender oil.

Another embodiment comprises cyclosporin A, Lipopeg 4-DO®, and Lavender oil.

Another embodiment comprises cyclosporin A, Lipopeg 4-DS®, and Lavender oil.

Another embodiment comprises cyclosporin A, Lipovol GTB®, and Lavender oil.

Another embodiment comprises cyclosporin A, Lonzest SML-20®, and Lavender oil.

Another embodiment comprises cyclosporin A, Lonzest SMO-20®, and Lavender oil.

Another embodiment comprises cyclosporin A, Lonzest SMS-20®, and Lavender oil.

Another embodiment comprises cyclosporin A, Lonzest STO-20®, and Lavender oil.

Another embodiment comprises cyclosporin A, Lonzest STS-20®, and Lavender oil.

Another embodiment comprises cyclosporin A, Lumulse GR-40®, and Lavender oil.

Another embodiment comprises cyclosporin A, Lumulse GRH-40®, and Lavender oil.

Another embodiment comprises cyclosporin A, Lumulse POE (7) GML®, and Lavender oil.

Another embodiment comprises cyclosporin A, Lumulse POE (12) Glyc®, and Lavender oil.

Another embodiment comprises cyclosporin A, Lumulse POE (40) MS KP®, and Lavender oil.

Another embodiment comprises cyclosporin A, Marlowet 4750®, and Lavender oil.

Another embodiment comprises cyclosporin A, Marlowet LVS®, and Lavender oil.

Another embodiment comprises cyclosporin A, Marlowet R 11®, and Lavender oil.

Another embodiment comprises cyclosporin A, Marlowet R 40®, and Lavender oil.

Another embodiment comprises cyclosporin A, Mazol 80 MGK®, and Lavender oil.

Another embodiment comprises cycldsporin A, Nonionic Emulsifier T-9®, and Lavender oil.

Another embodiment comprises cyclosporin A, Oronal LCG®, and Lavender oil.

Another embodiment comprises cyclosporin A, Polyderm PPI-CO-200®, and Lavender oil.

Another embodiment comprises cyclosporin A, Polyderm PPI-CO-40®, and Lavender oil.

Another embodiment comprises cyclosporin A, Rewoderm LI 520-70®, and Lavender oil.

Another embodiment comprises cyclosporin A, Ritapeg 150 DS®, and Lavender oil.

Another embodiment comprises cyclosporin A, Softigen 767®, and Lavender oil.

Another embodiment comprises cyclosporin A, Surfactol 318®, and Lavender oil.

Another embodiment comprises cyclosporin A, Surfactol 365®, and Lavender oil.

Another embodiment comprises cyclosporin A, Syn Lube 107®, and Lavender oil.

Another embodiment comprises cyclosporin A, Syn Lube 728®, and Lavender oil.

Another embodiment comprises cyclosporin A, Syn Lube 1632H®, and Lavender oil.

Another embodiment comprises cyclosporin A, Syn Lube 6277-A®, and Lavender oil.

Another embodiment comprises cyclosporin A, T-Det C-20®, and Lavender oil.

Another embodiment comprises cyclosporin A, T-Det C-40®, and Lavender oil.

Another embodiment comprises cyclosporin A, Tally 100 Plus®, and Lavender oil.

Another embodiment comprises cyclosporin A, Uniperol EL®, and Lavender oil.

Another embodiment comprises cyclosporin A, a Fatty Ester, and Lavender oil.

Another embodiment comprises cyclosporin A, Actralube-Syn 147®, and Lavender oil.

Another embodiment comprises cyclosporin A, Atlas G-1556®, and Lavender oil.

Another embodiment comprises cyclosporin A, Atlas G-1564®, and Lavender oil.

Another embodiment comprises cyclosporin A, Atlasol Base Oil S®, and Lavender oil.

Another embodiment comprises cyclosporin A, Base ML®, and Lavender oil.

Another embodiment comprises cyclosporin A, Base MT®, and Lavender oil.

Another embodiment comprises cyclosporin A, Cerasynt 303®, and Lavender oil.

Another embodiment comprises cyclosporin A, Dermol 1012®, and Lavender oil.

Another embodiment comprises cyclosporin A, Kemester 4000®, and Lavender oil.

Another embodiment comprises cyclosporin A, Lactipol S®, and Lavender oil.

Another embodiment comprises cyclosporin A, Magrabar PGO®, and Lavender oil.

Another embodiment comprises cyclosporin A, Mayco Base BFO®, and Lavender oil.

Another embodiment comprises cyclosporin A, Methyl Linoleate®, and Lavender oil.

Another embodiment comprises cyclosporin A, Pationic 122A®, and Lavender oil.

Another embodiment comprises cyclosporin A, Pationic 138C®, and Lavender oil.

Another embodiment comprises cyclosporin A, Pationic CSL®, and Lavender oil.

Another embodiment comprises cyclosporin A, Pationic ISL®, and Lavender oil.

Another embodiment comprises cyclosporin A, Pationic SBL®, and Lavender oil.

Another embodiment comprises cyclosporin A, Pationic SSL®, and Lavender oil.

Another embodiment comprises cyclosporin A, Ritasol®, and Lavender oil.

Another embodiment comprises cyclosporin A, Tego Alkanol CS 20®, and Lavender oil.

Another embodiment comprises cyclosporin A, Tego Alkanol L23 P®, and Lavender oil.

Another embodiment comprises cyclosporin A, Tego Alkanol S2®, and Lavender oil.

Another embodiment comprises cyclosporin A, Tego Alkanol S20 P®, and Lavender oil.

Another embodiment comprises cyclosporin A, Triemulsifier 600 MS®, and Lavender oil.

Another embodiment comprises cyclosporin A, a Glycerol Esters, and Lavender oil.

Another embodiment comprises cyclosporin A, Agro #9 Wint SBO®, and Lavender oil.

Another embodiment comprises cyclosporin A, Ahcovel Base 700®, and Lavender oil.

Another embodiment comprises cyclosporin A, Aldo HMS FG®, and Lavender oil.

Another embodiment comprises cyclosporin A, Aldo MLD®, and Lavender oil.

Another embodiment comprises cyclosporin A, Aldo MLD FG®, and Lavender oil.

Another embodiment comprises cyclosporin A, Aldo MO FG®, and Lavender oil.

Another embodiment comprises cyclosporin A, Aldo MS®, and Lavender oil.

Another embodiment comprises cyclosporin A, Aldo MS FG®, and Lavender oil.

Another embodiment comprises cyclosporin A, Aldo MS LG FG®, and Lavender oil.

Another embodiment comprises cyclosporin A, Aldo MSD®, and Lavender oil.

Another embodiment comprises cyclosporin A, Aldo MSD FG®, and Lavender oil.

Another embodiment comprises cyclosporin A, Aldosperse O-20 FG®, and Lavender oil.

Another embodiment comprises cyclosporin A, Aldosperse TS-20 FG®, and Lavender oil.

Another embodiment comprises cyclosporin A, Aldosperse TS-40 FG®, and Lavender oil.

Another embodiment comprises cyclosporin A, Arlacel 165®, and Lavender oil.

Another embodiment comprises cyclosporin A, Arlacel 186®, and Lavender oil.

Another embodiment comprises cyclosporin A, Capmul GMO®, and Lavender oil.

Another embodiment comprises cyclosporin A, Capmul GMS®, and Lavender oil.

Another embodiment comprises cyclosporin A, Caprol 3GO®, and Lavender oil.

Another embodiment comprises cyclosporin A, Caprol 3GVS®, and Lavender oil.

Another embodiment comprises cyclosporin A, Caprol 6G2S®, and Lavender oil.

Another embodiment comprises cyclosporin A, Caprol 10G40®, and Lavender oil.

Another embodiment comprises cyclosporin A, Caprol 10G100®, and Lavender oil.

Another embodiment comprises cyclosporin A, Caprol ET®, and Lavender oil.

Another embodiment comprises cyclosporin A, Caprol PGE860®, and Lavender oil.

Another embodiment comprises cyclosporin A, Cerasynt 945®, and Lavender oil.

Another embodiment comprises cyclosporin A, Cerasynt GMS®, and Lavender oil.

Another embodiment comprises cyclosporin A, Cerasynt Q®, and Lavender oil.

Another embodiment comprises cyclosporin A, Cerasynt SD®, and Lavender oil.

Another embodiment comprises cyclosporin A, Cerasynt WM®, and Lavender oil.

Another embodiment comprises cyclosporin A, Chemsperse 14®, and Lavender oil.

Another embodiment comprises cyclosporin A, Cremophor GO-32®, and Lavender oil.

Another embodiment comprises cyclosporin A, Cremophor GS11®, and Lavender oil.

Another embodiment comprises cyclosporin A, Cremophor GS-32®, and Lavender oil.

Another embodiment comprises cyclosporin A, Cutina KD-16®, and Lavender oil.

Another embodiment comprises cyclosporin A, Dehymuls PGPH®, and Lavender oil.

Another embodiment comprises cyclosporin A, Dermol DGDIS®, and Lavender oil.

Another embodiment comprises cyclosporin A, Dermol DGMIS®, and Lavender oil.

Another embodiment comprises cyclosporin A, Dermol G-76®, and Lavender oil.

Another embodiment comprises cyclosporin A, Dermol G-7DI®, and Lavender oil.

Another embodiment comprises cyclosporin A, Dermol NGDI®, and Lavender oil.

Another embodiment comprises cyclosporin A, Dermolan GLH®, and Lavender oil.

Another embodiment comprises cyclosporin A, Drewmulse GMO®, and Lavender oil.

Another embodiment comprises cyclosporin A, Drewpol 3-5-M®, and Lavender oil.

Another embodiment comprises cyclosporin A, Durlac 100 W®, and Lavender oil.

Another embodiment comprises cyclosporin A, Dur-Lo®, and Lavender oil.

Another embodiment comprises cyclosporin A, Dynasan 118®, and Lavender oil.

Another embodiment comprises cyclosporin A, EC-25®, and Lavender oil.

Another embodiment comprises cyclosporin A, EM 40®, and Lavender oil.

Another embodiment comprises cyclosporin A, Emerest 2400®, and Lavender oil.

Another embodiment comprises cyclosporin A, Emerest 2452®, and Lavender oil.

Another embodiment comprises cyclosporin A, Empilan G-26®, and Lavender oil.

Another embodiment comprises cyclosporin A, Genapol TSM®, and Lavender oil.

Another embodiment comprises cyclosporin A, Hostacerin DGI®, and Lavender oil.

Another embodiment comprises cyclosporin A, Hostacerin DGL®, and Lavender oil.

Another embodiment comprises cyclosporin A, Hostacerin DGMS®, and Lavender oil.

Another embodiment comprises cyclosporin A, Hostacerin DGSB®, and Lavender oil.

Another embodiment comprises cyclosporin A, Ice No. 2®, and Lavender oil.

Another embodiment comprises cyclosporin A, Imwitor 742®, and Lavender oil.

Another embodiment comprises cyclosporin A, Imwitor 780 K®, and Lavender oil.

Another embodiment comprises cyclosporin A, Imwitor 960 Flakes®, and Lavender oil.

Another embodiment comprises cyclosporin A, Isolan GI 34®, and Lavender oil.

Another embodiment comprises cyclosporin A, Isolan GO 33®, and Lavender-oil.

Another embodiment comprises cyclosporin A, Kemester 1000®, and Lavender oil.

Another embodiment comprises cyclosporin A, Kemester 2000®, and Lavender oil.

Another embodiment comprises cyclosporin A, Kemester 2000®, and Lavender oil.

Another embodiment comprises cyclosporin A, Kemester 6000SE®, and Lavender oil.

Another embodiment comprises cyclosporin A, Lamecreme DGE 18®, and Lavender oil.

Another embodiment comprises cyclosporin A, Lexemul 515®, and Lavender oil.

Another embodiment comprises cyclosporin A, Lexemul 561®, and Lavender oil.

Another embodiment comprises cyclosporin A, Lexemul AR®, and Lavender oil.

Another embodiment comprises cyclosporin A, Lexemul AS®, and Lavender oil.

Another embodiment comprises cyclosporin A, Lexemul GDL®, and Lavender oil.

Another embodiment comprises cyclosporin A, Lexemul T®, and Lavender oil.

Another embodiment comprises cyclosporin A, Lipomulse 165®, and Lavender oil.

Another embodiment comprises cyclosporin A, Lumulse GML K®, and Lavender oil.

Another embodiment comprises cyclosporin A, Lumulse GMO K®, and Lavender oil.

Another embodiment comprises cyclosporin A, Lumulse GMR K®, and Lavender oil.

Another embodiment comprises cyclosporin A, Lumulse GMT K®, and Lavender oil.

Another embodiment comprises cyclosporin A, Magrabar GMC®, and Lavender oil.

Another embodiment comprises cyclosporin A, Magrabar GMO-CK®, and Lavender oil.

Another embodiment comprises cyclosporin A, Magrabar MDG-5050®, and Lavender oil.

Another embodiment comprises cyclosporin A, Magrabar PGO-315®, and Lavender oil.

Another embodiment comprises cyclosporin A, Magrabar PGO-1010®, and Lavender oil.

Another embodiment comprises cyclosporin A, Mazol 300K®, and Lavender oil.

Another embodiment comprises cyclosporin A, Mazol GMO-K®, and Lavender oil.

Another embodiment comprises cyclosporin A, Mazol GMS-K®, and Lavender oil.

Another embodiment comprises cyclosporin A, Mazol PG031 -K®, and Lavender oil.

Another embodiment comprises cyclosporin A, Miglyol 812®, and Lavender oil.

Another embodiment comprises cyclosporin A, Norfox 165C®, and Lavender oil.

Another embodiment comprises cyclosporin A, Schercemol GMIS®, and Lavender oil.

Another embodiment comprises cyclosporin A, Tegin®, and Lavender oil.

Another embodiment comprises cyclosporin A, Tegin 4100 Pellets®, and Lavender oil.

Another embodiment comprises cyclosporin A, Tegin M Pellets®, and Lavender oil.

Another embodiment comprises cyclosporin A, Tegin OV®, and Lavender oil.

Another embodiment comprises cyclosporin A, Teginacid H®, and Lavender oil.

Another embodiment comprises cyclosporin A, Tego Cosmo P813®, and Lavender oil.

Another embodiment comprises cyclosporin A, Wickenol 535®, and Lavender oil.

Another embodiment comprises cyclosporin A, Witconol 14®, and Lavender oil.

Another embodiment comprises cyclosporin A, Witconol 14®, and Lavender oil.

Another embodiment comprises cyclosporin A, Witconol 14F®, and Lavender oil.

Another embodiment comprises cyclosporin A, Witconol 18L®, and Lavender oil.

Another embodiment comprises cyclosporin A, Witconol GOT®, and Lavender oil.

Another embodiment comprises cyclosporin A, Witconol MST®, and Lavender oil.

Another embodiment comprises cyclosporin A, Witconol RHT®, and Lavender oil.

Another embodiment comprises cyclosporin A, a Glycol Ester, and Lavender oil.

Another embodiment comprises cyclosporin A, Alkamuls 600 DO®, and Lavender oil.

Another embodiment comprises cyclosporin A, Alkamuls SEG®, and Lavender oil.

Another embodiment comprises cyclosporin A, Atlas EM-2®, and Lavender oil.

Another embodiment comprises cyclosporin A, Cerasynt IP®, and Lavender oil.

Another embodiment comprises cyclosporin A, Cerasynt M®, and Lavender oil.

Another embodiment comprises cyclosporin A, Cerasynt MN®, and Lavender oil.

Another embodiment comprises cyclosporin A, Cerasynt PA®, and Lavender oil.

Another embodiment comprises cyclosporin A, Chemsperse EGDS®, and Lavender oil.

Another embodiment comprises cyclosporin A, Chemsperse EGMS®, and Lavender oil.

Another embodiment comprises cyclosporin A, Colonial Monolaurin®, and Lavender oil.

Another embodiment comprises cyclosporin A, DeMuls SGE-95®, and Lavender oil.

Another embodiment comprises cyclosporin A, Eccoterge 200®, and Lavender oil.

Another embodiment comprises cyclosporin A, Emerest 2380®, and Lavender oil.

Another embodiment comprises cyclosporin A, Ethox 2610®, and Lavender oil.

Another embodiment comprises cyclosporin A, Ethox DO-9®, and Lavender oil.

Another embodiment comprises cyclosporin A, Ethox DO-14®, and Lavender oil.

Another embodiment comprises cyclosporin A, Ethox SO-9®, and Lavender oil.

Another embodiment comprises cyclosporin A, Fizul MD-318®, and Lavender oil.

Another embodiment comprises cyclosporin A, Genapol EGDS-VHP®, and Lavender oil.

Another embodiment comprises cyclosporin A, Genapol TS Powder®, and Lavender oil.

Another embodiment comprises cyclosporin A, Hostacerin WO®, and Lavender oil.

Another embodiment comprises cyclosporin A, Inversol 140®, and Lavender oil.

Another embodiment comprises cyclosporin A, Kemester 104®, and Lavender oil.

Another embodiment comprises cyclosporin A, Kemester 205®, and Lavender oil.

Another embodiment comprises cyclosporin A, Kemester 226®, and Lavender oil.

Another embodiment comprises cyclosporin A, Kemester 5221SE®, and Lavender oil.

Another embodiment comprises cyclosporin A, Kemester EGDS®, and Lavender oil.

Another embodiment comprises cyclosporin A, Lexemul EGDS®, and Lavender oil.

Another embodiment comprises cyclosporin A, Lexemul EGMS®, and Lavender oil.

Another embodiment comprises cyclosporin A, Lexemul P®, and Lavender oil.

Another embodiment comprises cyclosporin A, Lipo DGLS®, and Lavender oil.

Another embodiment comprises cyclosporin A, Lipo EGDS®, and Lavender oil.

Another embodiment comprises cyclosporin A, Lipo PGMS®, and Lavender oil.

Another embodiment comprises cyclosporin A, Liposorb S-4®, and Lavender oil.

Another embodiment comprises cyclosporin A, Liposorb TO-20®, and Lavender oil.

Another embodiment comprises cyclosporin A, Lumulse PGO®, and Lavender oil.

Another embodiment comprises cyclosporin A, Mackester EGDS®, and Lavender oil.

Another embodiment comprises cyclosporin A, Mackester EGMS®, and Lavender oil.

Another embodiment comprises cyclosporin A, Mackester GSTP®, and Lavender oil.

Another embodiment comprises cyclosporin A, Mackester Series®, and Lavender oil.

Another embodiment comprises cyclosporin A, Magrabar PDG-50®, and Lavender oil.

Another embodiment comprises cyclosporin A, Mapeg 6000 DS®, and Lavender oil.

Another embodiment comprises cyclosporin A, Marlowet 4702®, and Lavender oil.

Another embodiment comprises cyclosporin A, Monalube 305®, and Lavender oil.

Another embodiment comprises cyclosporin A, Monalube 310®, and Lavender oil.

Another embodiment comprises cyclosporin A, Monalube 315®, and Lavender oil.

Another embodiment comprises cyclosporin A, Monalube 320®, and Lavender oil.

Another embodiment comprises cyclosporin A, Monalube 325®, and Lavender oil.

Another embodiment comprises cyclosporin A, Monalube 330®, and Lavender oil.

Another embodiment comprises cyclosporin A, Naturechem PGHS®, and Lavender oil.

Another embodiment comprises cyclosporin A, Polycastorol PLO-840®, and Lavender oil.

Another embodiment comprises cyclosporin A, Polytex 10M®, and Lavender oil.

Another embodiment comprises cyclosporin A, Ritasynt IP®, and Lavender oil.

Another embodiment comprises cyclosporin A, Ross Chem PEG 600 DT®, and Lavender oil.

Another embodiment comprises cyclosporin A, Schercemol PGMS®, and Lavender oil.

Another embodiment comprises cyclosporin A, Sponto H-44C®, and Lavender oil.

Another embodiment comprises cyclosporin A, Tegin G®, and Lavender oil.

Another embodiment comprises cyclosporin A, Witbreak DGE-182®, and Lavender oil.

Another embodiment comprises cyclosporin A, Witbreak DGE-182®, and Lavender oil.

Another embodiment comprises cyclosporin A, Witbreak DRA-21®, and Lavender oil.

Another embodiment comprises cyclosporin A, Witbreak DRA-21®, and Lavender oil.

Another embodiment comprises cyclosporin A, Witbreak DRA-50®, and Lavender oil.

Another embodiment comprises cyclosporin A, Witbreak DRA-50®, and Lavender oil.

Another embodiment comprises cyclosporin A, Witconol F26-46®, and Lavender oil.

Another embodiment comprises cyclosporin A, Witconol H-32®, and Lavender oil.

Another embodiment comprises cyclosporin A, Witconol H-33®, and Lavender oil.

Another embodiment comprises cyclosporin A, Witconol H-35A®, and Lavender oil.

Another embodiment comprises cyclosporin A, Witconol RHP®, and Lavender oil.

Another embodiment comprises cyclosporin A, a Lanolin-based Derivative, and Lavender oil.

Another embodiment comprises cyclosporin A, Amerchol CAB®, and Lavender oil.

Another embodiment comprises cyclosporin A, Amerchol L-101®, and Lavender oil.

Another embodiment comprises cyclosporin A, Amerlate LFA-LO®, and Lavender oil.

Another embodiment comprises cyclosporin A, Amerlate P®, and Lavender oil.

Another embodiment comprises cyclosporin A, Barre Common Degras®, and Lavender oil.

Another embodiment comprises cyclosporin A, Cholesterol NF®, and Lavender oil.

Another embodiment comprises cyclosporin A, Crodalan AWS®, and Lavender oil.

Another embodiment comprises cyclosporin A, Crodalan LA®, and Lavender oil.

Another embodiment comprises cyclosporin A, Emery 1650®, and Lavender oil.

Another embodiment comprises cyclosporin A, Emery 1650®, and Lavender oil.

Another embodiment comprises cyclosporin A, Emery 1740®, and Lavender oil.

Another embodiment comprises cyclosporin A, Emery 1740®, and Lavender oil.

Another embodiment comprises cyclosporin A, Forlan 500®, and Lavender oil.

Another embodiment comprises cyclosporin A, Forlan L®, and Lavender oil.

Another embodiment comprises cyclosporin A, Laneto 50®, and Lavender oil.

Another embodiment comprises cyclosporin A, Laneto 100®, and Lavender oil.

Another embodiment comprises cyclosporin A, Laneto AWS®, and Lavender oil.

Another embodiment comprises cyclosporin A, Lanfrax 1776®, and Lavender oil.

Another embodiment comprises cyclosporin A, Lanfrax 1776®, and Lavender oil.

Another embodiment comprises cyclosporin A, Lanogel 21®, and Lavender oil.

Another embodiment comprises cyclosporin A, Lipolan®, and Lavender oil.

Another embodiment comprises cyclosporin A, Lipolan 31®, and Lavender oil.

Another embodiment comprises cyclosporin A, OHlan®, and Lavender oil.

Another embodiment comprises cyclosporin A, Polychol 5®, and Lavender oil.

Another embodiment comprises cyclosporin A, Polychol 15®, and Lavender oil.

Another embodiment comprises cyclosporin A, Ritacetyl®, and Lavender oil.

Another embodiment comprises cyclosporin A, Ritachol®, and Lavender oil.

Another embodiment comprises cyclosporin A, Ritahydrox®, and Lavender oil.

Another embodiment comprises cyclosporin A, Ritalafa®, and Lavender oil.

Another embodiment comprises cyclosporin A, Ritalan®, and Lavender oil.

Another embodiment comprises cyclosporin A, Ritalan AWS®, and Lavender oil.

Another embodiment comprises cyclosporin A, Ritalan C®, and Lavender oil.

Another embodiment comprises cyclosporin A, Ritawax®, and Lavender oil.

Another embodiment comprises cyclosporin A, Ritawax AEO®, and Lavender oil.

Another embodiment comprises cyclosporin A,-Ritawax ALA®, and Lavender oil.

Another embodiment comprises cyclosporin A, Solan/Solan 50/Super Solan®, and Lavender oil.

Another embodiment comprises cyclosporin A, Super Hartolan/Hartolan®, and Lavender oil.

Another embodiment comprises cyclosporin A, Supersat AWS-4®, and Lavender oil.

Another embodiment comprises cyclosporin A, Supersat AWS-24®, and Lavender oil.

Another embodiment comprises cyclosporin A, a Lecithin or Lecithin Derivative, and Lavender oil.

Another embodiment comprises cyclosporin A, Alcolec®, and Lavender oil.

Another embodiment comprises cyclosporin A, Lecithin®, and Lavender oil.

Another embodiment comprises cyclosporin A, Lexin K®, and Lavender oil.

Another embodiment comprises cyclosporin A, Natipide®, and Lavender oil.

Another embodiment comprises cyclosporin A, a Lignin or Lignin Derivative, and Lavender oil.

Another embodiment comprises cyclosporin A, Diwatex XP 9®, and Lavender oil.

Another embodiment comprises cyclosporin A, Dynasperse LCD®, and Lavender oil.

Another embodiment comprises cyclosporin A, Indulin SAL®, and Lavender oil.

Another embodiment comprises cyclosporin A, Indulin W-1®, and Lavender oil.

Another embodiment comprises cyclosporin A, Indulin W-5®, and Lavender oil.

Another embodiment comprises cyclosporin A, Lignosol FTA®, and Lavender oil.

Another embodiment comprises cyclosporin A, Lignosol SFX-65®, and Lavender oil.

Another embodiment comprises cyclosporin A, Marasperse 52 CP®, and Lavender oil.

Another embodiment comprises cyclosporin A, Marasperse AG®, and Lavender oil.

Another embodiment comprises cyclosporin A, Marasperse CBOS-4®, and Lavender oil.

Another embodiment comprises cyclosporin A, Ufoxane 2®, and Lavender oil.

Another embodiment comprises cyclosporin A, a Methyl Ester, and Lavender oil.

Another embodiment comprises cyclosporin A, E.B. Cleaner AK®, and Lavender oil.

Another embodiment comprises cyclosporin A, Oleocal ME-70®, and Lavender oil.

Another embodiment comprises cyclosporin A, Trade Name Company®, and Lavender oil.

Another embodiment comprises cyclosporin A, Methyl Esters (cont'd)®, and Lavender oil.

Another embodiment comprises cyclosporin A, Oleocal ME-92®, and Lavender oil.

Another embodiment comprises cyclosporin A, Oleocal ME-112®, and Lavender oil.

Another embodiment comprises cyclosporin A, Oleocal ME-130®, and Lavender oil.

Another embodiment comprises cyclosporin A, a Monoglyceride or a Derivative thereof, and Lavender oil.

Another embodiment comprises cyclosporin A, Dynacet 211®, and Lavender oil.

Another embodiment comprises cyclosporin A, Hetsorb S-20®, and Lavender oil.

Another embodiment comprises cyclosporin A, Imwitor 191®, and Lavender oil.

Another embodiment comprises cyclosporin A, Imwitor 370®, and Lavender oil.

Another embodiment comprises cyclosporin A, Imwitor 375®, and Lavender oil.

Another embodiment comprises cyclosporin A, Imwitor 900®, and Lavender oil.

Another embodiment comprises cyclosporin A, Imwitor 945®, and Lavender oil.

Another embodiment comprises cyclosporin A, Imwitor 2020®, and Lavender oil.

Another embodiment comprises cyclosporin A, Kemester 5500®, and Lavender oil.

Another embodiment comprises cyclosporin A, Kemester 6000®, and Lavender oil.

Another embodiment comprises cyclosporin A, Magrabar GMC®, and Lavender oil.

Another embodiment comprises cyclosporin A, Magrabar GMO-CK®, and Lavender oil.

Another embodiment comprises cyclosporin A, Magrabar GPC-10®, and Lavender oil.

Another embodiment comprises cyclosporin A, Magrabar MDG-5050®, and Lavender oil.

Another embodiment comprises cyclosporin A, Monalube 335®, and Lavender oil.

Another embodiment comprises cyclosporin A, Monoglycerides Glyceryl Monestearate Archer Daniels Midland Company®, and Lavender oil.

Another embodiment comprises cyclosporin A, Rita GMS®, and Lavender oil.

Another embodiment comprises cyclosporin A, Ritamulse SCG®, and Lavender oil.

Another embodiment comprises cyclosporin A, Softigen 701®, and Lavender oil.

Another embodiment comprises cyclosporin A, Tally 100 Plus®, and Lavender oil.

Another embodiment comprises cyclosporin A, a Polyethylene Glycol, and Lavender oil.

Another embodiment comprises cyclosporin A, Emulgade PL 68/50®, and Lavender oil.

Another embodiment comprises cyclosporin A, Lumulse PEG®, and Lavender oil.

Another embodiment comprises cyclosporin A, Rhodasurf PEG-400®, and Lavender oil.

Another embodiment comprises cyclosporin A, Rhodasurf PEG-600®, and Lavender oil.

Another embodiment comprises cyclosporin A, Witconol PEG-400®, and Lavender oil.

Another embodiment comprises cyclosporin A, a Polymeric Surfactant, and Lavender oil.

Another embodiment comprises cyclosporin A, Acritamer PNC-EG®, and Lavender oil.

Another embodiment comprises cyclosporin A, Ag-Rho DEP-775®, and Lavender oil.

Another embodiment comprises cyclosporin A, APG 325N Glycoside®, and Lavender oil.

Another embodiment comprises cyclosporin A, Aristoflex AVC®, and Lavender oil.

Another embodiment comprises cyclosporin A, Aristoflex HMB®, and Lavender oil.

Another embodiment comprises cyclosporin A, Burco NPS-225®, and Lavender oil.

Another embodiment comprises cyclosporin A, Burco NPS-816®, and Lavender oil.

Another embodiment comprises cyclosporin A, Chemccinate 5603®, and Lavender oil.

Another embodiment comprises cyclosporin A, Cosmedia Guar C-261 N®, and Lavender oil.

Another embodiment comprises cyclosporin A, Gantrez S-95®, and Lavender oil.

Another embodiment comprises cyclosporin A, Glucopon 220 UP®, and Lavender oil.

Another embodiment comprises cyclosporin A, Glucopon 225 DK®, and Lavender oil.

Another embodiment comprises cyclosporin A, Glucopon 425 N®, and Lavender oil.

Another embodiment comprises cyclosporin A, Glucopon 600 UP®, and Lavender oil.

Another embodiment comprises cyclosporin A, Glucopon 625 UP®, and Lavender oil.

Another embodiment comprises cyclosporin A, Pemulen 1621®, and Lavender oil.

Another embodiment comprises cyclosporin A, Pemulen 1622®, and Lavender oil.

Another embodiment comprises cyclosporin A, Pemulen TR-1®, and Lavender oil.

Another embodiment comprises cyclosporin A, Pemulen TR-2®, and Lavender oil.

Another embodiment comprises cyclosporin A, Plantacare 818®, and Lavender oil.

Another embodiment comprises cyclosporin A, Plantapon LGC Sorb®, and Lavender oil.

Another embodiment comprises cyclosporin A, Plantaren 1200N®, and Lavender oil.

Another embodiment comprises cyclosporin A, Plantaren 2000N®, and Lavender oil.

Another embodiment comprises cyclosporin A, Viscolam AT 64®, and Lavender oil.

Another embodiment comprises cyclosporin A, Viscolam AT 64P®, and Lavender oil.

Another embodiment comprises cyclosporin A, Viscolam AT 100®, and Lavender oil.

Another embodiment comprises cyclosporin A, Viscolam MAC 7®, and Lavender oil.

Another embodiment comprises cyclosporin A, Viscolam SMC 20®, and Lavender oil.

Another embodiment comprises cyclosporin A, Witbreak RTC-323®, and Lavender oil.

Another embodiment comprises cyclosporin A, WSI 3700®, and Lavender oil.

Another embodiment comprises cyclosporin A, a Propoxylated or Ethoxylated Fatty Acid, Alcohol, or Alkyl Phenol, and Lavender oil.

Another embodiment comprises cyclosporin A, Antarox AA-60®, and Lavender oil.

Another embodiment comprises cyclosporin A, Antarox LF-224®, and Lavender oil.

Another embodiment comprises cyclosporin A, Burcomul DFE-45®, and Lavender oil.

Another embodiment comprises cyclosporin A, Burcoterge LFE-1000®, and Lavender oil.

Another embodiment comprises cyclosporin A, Chemal LF-25B®, and Lavender oil.

Another embodiment comprises cyclosporin A, Chemal LF-40B®, and Lavender oil.

Another embodiment comprises cyclosporin A, Dehypon LS-36®, and Lavender oil.

Another embodiment comprises cyclosporin A, Dehypon LS-36®, and Lavender oil.

Another embodiment comprises cyclosporin A, Dehypon LS-54®, and Lavender oil.

Another embodiment comprises cyclosporin A, Dehypon LS-54®, and Lavender oil.

Another embodiment comprises cyclosporin A, Delonic 100 VLF®, and Lavender oil.

Another embodiment comprises cyclosporin A, Delonic LF-60 MOD®, and Lavender oil.

Another embodiment comprises cyclosporin A, Empiderm B®, and Lavender oil.

Another embodiment comprises cyclosporin A, Ethylan 1206®, and Lavender oil.

Another embodiment comprises cyclosporin A, Ethylan NS-500K®, and Lavender oil.

Another embodiment comprises cyclosporin A, Ethylan NS-500LQ®, and Lavender oil.

Another embodiment comprises cyclosporin A, Genapol 1392®, and Lavender oil.

Another embodiment comprises cyclosporin A, Genapol 2317®, and Lavender oil.

Another embodiment comprises cyclosporin A, Genapol 26EP710®, and Lavender oil.

Another embodiment comprises cyclosporin A, Genapol EP 1022®, and Lavender oil.

Another embodiment comprises cyclosporin A, Genapol EP 1024®, and Lavender oil.

Another embodiment comprises cyclosporin A, Genapol EP 6068®, and Lavender oil.

Another embodiment comprises cyclosporin A, Genapol NP915®, and Lavender oil.

Another embodiment comprises cyclosporin A, Kieralon MFB®, and Lavender oil.

Another embodiment comprises cyclosporin A, Lumisolve CSA-80 V®, and Lavender oil.

Another embodiment comprises cyclosporin A, Marlowet 5001®, and Lavender oil.

Another embodiment comprises cyclosporin A, Marlox FK 64®, and Lavender oil.

Another embodiment comprises cyclosporin A, Marlox MO 124®, and Lavender oil.

Another embodiment comprises cyclosporin A, Marlox S 58®, and Lavender oil.

Another embodiment comprises cyclosporin A, Nonatell 1003®, and Lavender oil.

Another embodiment comprises cyclosporin A, Nonatell 1038®, and Lavender oil.

Another embodiment comprises cyclosporin A, Nonatell 1052®, and Lavender oil.

Another embodiment comprises cyclosporin A, Nonatell 1061®, and Lavender oil.

Another embodiment comprises cyclosporin A, Nonatell 1075®, and Lavender oil.

Another embodiment comprises cyclosporin A, Nonatell 1088®, and Lavender oil.

Another embodiment comprises cyclosporin A, Nonatell 1123®, and Lavender oil.

Another embodiment comprises cyclosporin A, Nonatell 1153®, and Lavender oil.

Another embodiment comprises cyclosporin A, Nonatell 1161®, and Lavender oil.

Another embodiment comprises cyclosporin A, Nonatell 1172®, and Lavender oil.

Another embodiment comprises cyclosporin A, Nonatell 1181®, and Lavender oil.

Another embodiment comprises cyclosporin A, Norfox 36®, and Lavender oil.

Another embodiment comprises cyclosporin A, Procetyl AWS®, and Lavender oil.

Another embodiment comprises cyclosporin A, Sandoxylate SX 412®, and Lavender oil.

Another embodiment comprises cyclosporin A, Sandoxylate SX 418®, and Lavender oil.

Another embodiment comprises cyclosporin A, Surfonic JL-80X®, and Lavender oil.

Another embodiment comprises cyclosporin A, Surfonic JL-80X-B1®, and Lavender oil.

Another embodiment comprises cyclosporin A, Surfonic L4-29X®, and Lavender oil.

Another embodiment comprises cyclosporin A, Surfonic LF®, and Lavender oil.

Another embodiment comprises cyclosporin A, T-Det A826®, and Lavender oil.

Another embodiment comprises cyclosporin A, T-Det LF-416®, and Lavender oil.

Another embodiment comprises cyclosporin A, Tergitol Min-Foam 1X®, and Lavender oil.

Another embodiment comprises cyclosporin A, Tergitol Min-Foam 2X®, and Lavender oil.

Another embodiment comprises cyclosporin A, Triton CF-21®, and Lavender oil.

Another embodiment comprises cyclosporin A, Triton CF-76®, and Lavender oil.

Another embodiment comprises cyclosporin A, Triton XL-80N®, and Lavender oil.

Another embodiment comprises cyclosporin A, Witconol NS-98®, and Lavender oil.

Another embodiment comprises cyclosporin A, Witconol NS-108LQ®, and Lavender oil.

Another embodiment comprises cyclosporin A, Witconol NS-145®, and Lavender oil.

Another embodiment comprises cyclosporin A, Witconol NS-179®, and Lavender oil.

Another embodiment comprises cyclosporin A, a Protein-based Surfactant, and Lavender oil.

Another embodiment comprises cyclosporin A, AminoFoam W®, and Lavender oil.

Another embodiment comprises cyclosporin A, Amiter LGOD-2®, and Lavender oil.

Another embodiment comprises cyclosporin A, Amiter LGS-2®, and Lavender oil.

Another embodiment comprises cyclosporin A, Amiter LGS-5®, and Lavender oil.

Another embodiment comprises cyclosporin A, Lamepon S®, and Lavender oil.

Another embodiment comprises cyclosporin A, Lamepon S®, and Lavender oil.

Another embodiment comprises cyclosporin A, Maypon 4C®, and Lavender oil.

Another embodiment comprises cyclosporin A, May-Tein C®, and Lavender oil.

Another embodiment comprises cyclosporin A, May-Tein CT®, and Lavender oil.

Another embodiment comprises cyclosporin A, May-Tein KTS®, and Lavender oil.

Another embodiment comprises cyclosporin A, May-Tein SY®, and Lavender oil.

Another embodiment comprises cyclosporin A, Plantapon S®, and Lavender oil.

Another embodiment comprises cyclosporin A, Proteol APL®, and Lavender oil.

Another embodiment comprises cyclosporin A, Proteol OAT®, and Lavender oil.

Another embodiment comprises cyclosporin A, Pyroter CPI-40®, and Lavender oil.

Another embodiment comprises cyclosporin A, Pyroter GPI-25®, and Lavender oil.

Another embodiment comprises cyclosporin A, Supro-Tein S®, and Lavender oil.

Another embodiment comprises cyclosporin A, Supro-Tein V®, and Lavender oil.

Another embodiment comprises cyclosporin A,® and Lavender oil.

Another embodiment comprises cyclosporin A, a Sarcosine Derivative®, and Lavender oil.

Another embodiment comprises cyclosporin A, Crodasinic LS-30®, and Lavender oil.

Another embodiment comprises cyclosporin A, Vanseal CS®, and Lavender oil.

Another embodiment comprises cyclosporin A, Vanseal LS®, and Lavender oil.

Another embodiment comprises cyclosporin A, Vanseal MS®, and Lavender oil.

Another embodiment comprises cyclosporin A, Vanseal NACS-30®, and Lavender oil.

Another embodiment comprises cyclosporin A, Vanseal NALS-95®, and Lavender oil.

Another embodiment comprises cyclosporin A, Vanseal OS®, and Lavender oil.

Another embodiment comprises cyclosporin A,® and Lavender oil.

Another embodiment comprises cyclosporin A, a Silicone-based Suriactant®, and Lavender oil.

Another embodiment comprises cyclosporin A, Abil-B-9950®, and Lavender oil.

Another embodiment comprises cyclosporin A, Abil Care 85®, and Lavender oil.

Another embodiment comprises cyclosporin A, Abil EM 90®, and Lavender oil.

Another embodiment comprises cyclosporin A, Abil EM 97®, and Lavender oil.

Another embodiment comprises cyclosporin A, Abil WE-09®, and Lavender oil.

Another embodiment comprises cyclosporin A, Dow Corning 1248 Fluid®, and Lavender oil.

Another embodiment comprises cyclosporin A, Dow Corning 3225C®, and Lavender oil.

Another embodiment comprises cyclosporin A, Dow Corning 5200®, and Lavender oil.

Another embodiment comprises cyclosporin A, Dow Corning Q4-3667®, and Lavender oil.

Another embodiment comprises cyclosporin A, Monasil PCA®, and Lavender oil.

Another embodiment comprises cyclosporin A, Monasil PDM®, and Lavender oil.

Another embodiment comprises cyclosporin A, Monasil PLN®, and Lavender oil.

Another embodiment comprises cyclosporin A, Polyderm PPI-SI-WS®, and Lavender oil.

Another embodiment comprises cyclosporin A, Troysol 380W®, and Lavender oil.

Another embodiment comprises cyclosporin A, Troysol S366®, and Lavender oil.

Another embodiment comprises cyclosporin A, a Sorbitan Derivative, and Lavender oil.

Another embodiment comprises cyclosporin A, Alkamuls SML®, and Lavender oil.

Another embodiment comprises cyclosporin A, Alkamuls SMO®, and Lavender oil.

Another embodiment comprises cyclosporin A, Alkamuls STO®, and Lavender oil.

Another embodiment comprises cyclosporin A, Arlacel 20®, and Lavender oil.

Another embodiment comprises cyclosporin A, Arlacel 40®, and Lavender oil.

Another embodiment comprises cyclosporin A, Arlacel 60®, and Lavender oil.

Another embodiment comprises cyclosporin A, Arlacel 80®, and Lavender oil.

Another embodiment comprises cyclosporin A, Arlacel C®, and Lavender oil.

Another embodiment comprises cyclosporin A, Armul 21®, and Lavender oil.

Another embodiment comprises cyclosporin A, Atlox 80®, and Lavender oil.

Another embodiment comprises cyclosporin A, Atlox 847®, and Lavender oil.

Another embodiment comprises cyclosporin A, Atlox 1045A®, and Lavender oil.

Another embodiment comprises cyclosporin A, Canarcel 20®, and Lavender oil.

Another embodiment comprises cyclosporin A, Canarcel 60®, and Lavender oil.

Another embodiment comprises cyclosporin A, Canarcel 80®, and Lavender oil.

Another embodiment comprises cyclosporin A, Canarcel TW 20®, and Lavender oil.

Another embodiment comprises cyclosporin A, Canarcel TW 60®, and Lavender oil.

Another embodiment comprises cyclosporin A, Canarcel TW 80®, and Lavender oil.

Another embodiment comprises cyclosporin A, Coladet BSB-P®, and Lavender oil.

Another embodiment comprises cyclosporin A, Customulse O-20®, and Lavender oil.

Another embodiment comprises cyclosporin A, Dehymuls E®, and Lavender oil.

Another embodiment comprises cyclosporin A, DeSotan SMO®, and Lavender oil.

Another embodiment comprises cyclosporin A, DeSotan SMO-20®, and Lavender oil.

Another embodiment comprises cyclosporin A, DeSotan SMT®, and Lavender oil.

Another embodiment comprises cyclosporin A, DeSotan SMT-20®, and Lavender oil.

Another embodiment comprises cyclosporin A, Durfax 60®, and Lavender oil.

Another embodiment comprises cyclosporin A, Durfax 65®, and Lavender oil.

Another embodiment comprises cyclosporin A, Durfax 80®, and Lavender oil.

Another embodiment comprises cyclosporin A, Durtan 60®, and Lavender oil.

Another embodiment comprises cyclosporin A, Durtan 65®, and Lavender oil.

Another embodiment comprises cyclosporin A, Liposorb L®, and Lavender oil.

Another embodiment comprises cyclosporin A, Liposorb L-10®, and Lavender oil.

Another embodiment comprises cyclosporin A, Liposorb L-20®, and Lavender oil.

Another embodiment comprises cyclosporin A, Liposorb O®, and Lavender oil.

Another embodiment comprises cyclosporin A, Liposorb O-20®, and Lavender oil.

Another embodiment comprises cyclosporin A, Liposorb P®, and Lavender oil.

Another embodiment comprises cyclosporin A, Liposorb P-20®, and Lavender oil.

Another embodiment comprises cyclosporin A, Liposorb S®, and Lavender oil.

Another embodiment comprises cyclosporin A, Liposorb S-20®, and Lavender oil.

Another embodiment comprises cyclosporin A, Liposorb SQO®, and Lavender oil.

Another embodiment comprises cyclosporin A, Liposorb TO®, and Lavender oil.

Another embodiment comprises cyclosporin A, Liposorb TS®, and Lavender oil.

Another embodiment comprises cyclosporin A, Liposorb TS-20®, and Lavender oil.

Another embodiment comprises cyclosporin A, Lumisorb PS®, and Lavender oil.

Another embodiment comprises cyclosporin A, Lumisorb SMO (T)®, and Lavender oil.

Another embodiment comprises cyclosporin A, Lumisorb SMS K®, and Lavender oil.

Another embodiment comprises cyclosporin A, Lumisorb SSO®, and Lavender oil.

Another embodiment comprises cyclosporin A, Lumisorb STS K®, and Lavender oil.

Another embodiment comprises cyclosporin A, Lumisorb STT®, and Lavender oil.

Another embodiment comprises cyclosporin A, Magrabar SMO®, and Lavender oil.

Another embodiment comprises cyclosporin A, Magrabar SMO-VEG®, and Lavender oil.

Another embodiment comprises cyclosporin A, Magrabar SMT®, and Lavender oil.

Another embodiment comprises cyclosporin A, Magrabar STO®, and Lavender oil.

Another embodiment comprises cyclosporin A, Miracare BC-27®, and Lavender oil.

Another embodiment comprises cyclosporin A, Ritabate 20®, and Lavender oil.

Another embodiment comprises cyclosporin A, Ritabate 40®, and Lavender oil.

Another embodiment comprises cyclosporin A, Ritabate 60®, and Lavender oil.

Another embodiment comprises cyclosporin A, Ritabate 80®, and Lavender oil.

Another embodiment comprises cyclosporin A, T-Maz®, and Lavender oil.

Another embodiment comprises cyclosporin A, Tego SML®, and Lavender oil.

Another embodiment comprises cyclosporin A, Tego SML 20®, and Lavender oil.

Another embodiment comprises cyclosporin A, Tego SMO 80 V®, and Lavender oil.

Another embodiment comprises cyclosporin A, Tego SMO V®, and Lavender oil.

Another embodiment comprises cyclosporin A, Tego SMS®, and Lavender oil.

Another embodiment comprises cyclosporin A, Tego STO V®, and Lavender oil.

Another embodiment comprises cyclosporin A, Tween 21®, and Lavender oil.

Another embodiment comprises cyclosporin A, Tween 40®, and Lavender oil.

Another embodiment comprises cyclosporin A, Tween 60®, and Lavender oil.

Another embodiment comprises cyclosporin A, Tween 60 K®, and Lavender oil.

Another embodiment comprises cyclosporin A, Tween 61®, and Lavender oil.

Another embodiment comprises cyclosporin A, Tween 65®, and Lavender oil.

Another embodiment comprises cyclosporin A, Tween 80®, and Lavender oil.

Another embodiment comprises cyclosporin A, Tween 80 K®, and Lavender oil.

Another embodiment comprises cyclosporin A, Tween 81®, and Lavender oil.

Another embodiment comprises cyclosporin A, Tween 85®, and Lavender oil.

Another embodiment comprises cyclosporin A, a Sucrose or Glucose Ester, or Derivative thereof, and Lavender oil.

Another embodiment comprises cyclosporin A, DeSulf GOS-P-60WCG®, and Lavender oil.

Another embodiment comprises cyclosporin A, Glucam E-20 Distearate®, and Lavender oil.

Another embodiment comprises cyclosporin A, Glucamate DOE-120®, and Lavender oil.

Another embodiment comprises cyclosporin A, Glucamate SSE-20®, and Lavender oil.

Another embodiment comprises cyclosporin A, Glucate DO®, and Lavender oil.

Another embodiment comprises cyclosporin A, Glucate SS®, and Lavender oil.

Another embodiment comprises cyclosporin A, Glucopon 425 UP®, and Lavender oil.

Another embodiment comprises cyclosporin A, Isolan IS®, and Lavender oil.

Another embodiment comprises cyclosporin A, Mazon 40®, and Lavender oil.

Another embodiment comprises cyclosporin A, Montanov 82®, and Lavender oil.

Another embodiment comprises cyclosporin A, Montanov 202®, and Lavender oil.

Another embodiment comprises cyclosporin A, Montanov S®, and Lavender oil.

Another embodiment comprises cyclosporin A, Rheozan®, and Lavender oil.

Another embodiment comprises cyclosporin A, Simulsol AS 48®, and Lavender oil.

Another embodiment comprises cyclosporin A, Simulsol SL 4®, and Lavender oil.

Another embodiment comprises cyclosporin A, Simulsol SL 10®, and Lavender oil.

Another embodiment comprises cyclosporin A, Simulsol SL 11W®, and Lavender oil.

Another embodiment comprises cyclosporin A, Simulsol SL 55®, and Lavender oil.

Another embodiment comprises cyclosporin A, Suga Nate 100 and 160®, and Lavender oil.

Another embodiment comprises cyclosporin A, Tego Care 450®, and Lavender oil.

Another embodiment comprises cyclosporin A, Tego Care CG 90®, and Lavender oil.

Another embodiment comprises cyclosporin A, Tego Care PS®, and Lavender oil.

Another embodiment comprises cyclosporin A, Tegosoft PSE 141 G®, and Lavender oil.

Another embodiment comprises cyclosporin A, Tegotens G 826®, and Lavender oil.

Another embodiment comprises cyclosporin A, Triton BG-10 (70%)®, and Lavender oil.

Another embodiment comprises cyclosporin A, Triton CG-110 (60%)®, and Lavender oil.

Another embodiment comprises cyclosporin A, Wickenol 545®, and Lavender oil.

Another embodiment comprises cyclosporin A, an alcohol, and Citronella oil.

Another embodiment comprises cyclosporin A, Diglycerol®, and Citronella oil.

Another embodiment comprises cyclosporin A, Hetoxide GT-80®, and Citronella oil.

Another embodiment comprises cyclosporin A, Lexemul BEO®, and Citronella oil.

Another embodiment comprises cyclosporin A, Polyglycerol-3®, and Citronella oil.

Another embodiment comprises cyclosporin A, Redicote E Series®, and Citronella oil.

Another embodiment comprises cyclosporin A, Simulsol OX 1005L®, and Citronella oil.

Another embodiment comprises cyclosporin A, Stanfax 567®, and Citronella oil.

Another embodiment comprises cyclosporin A, TA-1618®, and Citronella oil.

Another embodiment comprises cyclosporin A, Witconol H-31A®, and Citronella oil.

Another embodiment comprises cyclosporin A, an amine oxide, and Citronella oil.

Another embodiment comprises cyclosporin A, AO-405®, and Citronella oil.

Another embodiment comprises cyclosporin A, AO-455®, and Citronella oil.

Another embodiment comprises cyclosporin A, AO 728 Special®, and Citronella oil.

Another embodiment comprises cyclosporin A, Barlox 12®, and Citronella oil.

Another embodiment comprises cyclosporin A, Barlox 14®, and Citronella oil.

Another embodiment comprises cyclosporin A, Burcoxide Lo®, and Citronella oil.

Another embodiment comprises cyclosporin A, Caloxamine LO®, and Citronella oil.

Another embodiment comprises cyclosporin A, Chemoxide CAW®, and Citronella oil.

Another embodiment comprises cyclosporin A, Chemoxide LM-30®, and Citronella oil.

Another embodiment comprises cyclosporin A, Chemoxide LO®, and Citronella oil.

Another embodiment comprises cyclosporin A, Chemoxide MO®, and Citronella oil.

Another embodiment comprises cyclosporin A, Colalux CAO-35®, and Citronella oil.

Another embodiment comprises cyclosporin A, Colalux LO®, and Citronella oil.

Another embodiment comprises cyclosporin A, DeMox CAPO®, and Citronella oil.

Another embodiment comprises cyclosporin A, DeMox CSG-30®, and Citronella oil.

Another embodiment comprises cyclosporin A, DeMox LAO®, and Citronella oil.

Another embodiment comprises cyclosporin A, Emcol L®, and Citronella oil.

Another embodiment comprises cyclosporin A, Empigen OB®, and Citronella oil.

Another embodiment comprises cyclosporin A, Empigen OS/A®, and Citronella oil.

Another embodiment comprises cyclosporin A, Foamox CDO®, and Citronella oil.

Another embodiment comprises cyclosporin A, Foamox DMM®, and Citronella oil.

Another embodiment comprises cyclosporin A, Foamox DMS®, and Citronella oil.

Another embodiment comprises cyclosporin A, Genaminox KC®, and Citronella oil.

Another embodiment comprises cyclosporin A, Genaminox LA®, and Citronella oil.

Another embodiment comprises cyclosporin A, Hartofoam SAO®, and Citronella oil.

Another embodiment comprises cyclosporin A, Hartox DMCD®, and Citronella oil.

Another embodiment comprises cyclosporin A, Lipowax DAT®, and Citronella oil.

Another embodiment comprises cyclosporin A, Lipowax PB Pastilles®, and Citronella oil.

Another embodiment comprises cyclosporin A, Mackamine C8®, and Citronella oil.

Another embodiment comprises cyclosporin A, Mackamine C10®, and Citronella oil.

Another embodiment comprises cyclosporin A, Mackamine C14®, and Citronella oil.

Another embodiment comprises cyclosporin A, Mackamine CAO®, and Citronella oil.

Another embodiment comprises cyclosporin A, Mackamine CO®, and Citronella oil.

Another embodiment comprises cyclosporin A, Mackamine LO®, and Citronella oil.

Another embodiment comprises cyclosporin A, Mackamine O2®, and Citronella oil.

Another embodiment comprises cyclosporin A, Mackamine SAO®, and Citronella oil.

Another embodiment comprises cyclosporin A, Mackamine SO®, and Citronella oil.

Another embodiment comprises cyclosporin A, Mazox KCAO®, and Citronella oil.

Another embodiment comprises cyclosporin A, Monalac MO®, and Citronella oil.

Another embodiment comprises cyclosporin A, Norfox LDA®, and Citronella oil.

Another embodiment comprises cyclosporin A, Rhodamox LO®, and Citronella oil.

Another embodiment comprises cyclosporin A, Schercamox CAA®, and Citronella oil.

Another embodiment comprises cyclosporin A, Schercamox DMA®, and Citronella oil.

Another embodiment comprises cyclosporin A, Schercamox DML®, and Citronella oil.

Another embodiment comprises cyclosporin A, Schercamox DMM®, and Citronella oil.

Another embodiment comprises cyclosporin A, Schercamox DMS®, and Citronella oil.

Another embodiment comprises cyclosporin A, Tegotens DO®, and Citronella oil.

Another embodiment comprises cyclosporin A, Tomah AO-14-2®, and Citronella oil.

Another embodiment comprises cyclosporin A, Triaminox CDO®, and Citronella oil.

Another embodiment comprises cyclosporin A, a block polymer, and Citronella oil.

Another embodiment comprises cyclosporin A, AL 2070®, and Citronella oil.

Another embodiment comprises cyclosporin A, Antarox 17-R-2®, and Citronella oil.

Another embodiment comprises cyclosporin A, Antarox 25-R-2®, and Citronella oil.

Another embodiment comprises cyclosporin A, Antarox 31-R-1®, and Citronella oil.

Another embodiment comprises cyclosporin A, Antarox P-84®, and Citronella oil.

Another embodiment comprises cyclosporin A, Antarox P-104/H®, and Citronella oil.

Another embodiment comprises cyclosporin A, Arnox BP-Series®, and Citronella oil.

Another embodiment comprises cyclosporin A, Chemonic 435®, and Citronella oil.

Another embodiment comprises cyclosporin A, Chemonic D-25®, and Citronella oil.

Another embodiment comprises cyclosporin A, Chemonic PL Series®, and Citronella oil.

Another embodiment comprises cyclosporin A, Ethox L-121®, and Citronella oil.

Another embodiment comprises cyclosporin A, Ethox L-122®, and Citronella oil.

Another embodiment comprises cyclosporin A, Genapol PF-10®, and Citronella oil.

Another embodiment comprises cyclosporin A, Genapol PF-20®, and Citronella oil.

Another embodiment comprises cyclosporin A, Genapol PF-40A®, and Citronella oil.

Another embodiment comprises cyclosporin A, Norfox 2-LF®, and Citronella oil.

Another embodiment comprises cyclosporin A, Simulsol NW 342®, and Citronella oil.

Another embodiment comprises cyclosporin A, T-Det BP-1®, and Citronella oil.

Another embodiment comprises cyclosporin A, T-Det XD®, and Citronella oil.

Another embodiment comprises cyclosporin A, T-Det XH®, and Citronella oil.

Another embodiment comprises cyclosporin A, Triton CF-32®, and Citronella oil.

Another embodiment comprises cyclosporin A, Witconol 171®, and Citronella oil.

Another embodiment comprises cyclosporin A, Witconol 324®, and Citronella oil.

Another embodiment comprises cyclosporin A, Witconol 324D®, and Citronella oil.

Another embodiment comprises cyclosporin A, Witconol PD-2000®, and Citronella oil.

Another embodiment comprises cyclosporin A, a Carboxylated Alcohol or Alkylphenol Ethoxylate, and Citronella oil.

Another embodiment comprises cyclosporin A, Emcol CN-6®, and Citronella oil.

Another embodiment comprises cyclosporin A, Ethcarb®, and Citronella oil.

Another embodiment comprises cyclosporin A, Gemtex WNT-Conc®, and Citronella oil.

Another embodiment comprises cyclosporin A, Incrodet TD7-C®, and Citronella oil.

Another embodiment comprises cyclosporin A, Marlinat CM 105/80®, and Citronella oil.

Another embodiment comprises cyclosporin A, Marlowet 1072®, and Citronella oil.

Another embodiment comprises cyclosporin A, Marlowet 4530®, and Citronella oil.

Another embodiment comprises cyclosporin A, Marlowet 4530 LF®, and Citronella oil.

Another embodiment comprises cyclosporin A, Marlowet 4534®, and Citronella oil.

Another embodiment comprises cyclosporin A, Marlowet 4538®, and Citronella oil.

Another embodiment comprises cyclosporin A, Marlowet 4539®, and Citronella oil.

Another embodiment comprises cyclosporin A, Marlowet 4539 LF®, and Citronella oil.

Another embodiment comprises cyclosporin A, Marlowet 4541®, and Citronella oil.

Another embodiment comprises cyclosporin A, Miranate LEC-80®, and Citronella oil.

Another embodiment comprises cyclosporin A, Sandopan B®, and Citronella oil.

Another embodiment comprises cyclosporin A, Sandopan B Modified®, and Citronella oil.

Another embodiment comprises cyclosporin A, Sandopan LS-24 Gel®, and Citronella oil.

Another embodiment comprises cyclosporin A, Surfine T-A®, and Citronella oil.

Another embodiment comprises cyclosporin A, a Carboxylic Acid/Fatty Acid, and Citronella oil.

Another embodiment comprises cyclosporin A, Colaterge RAM®, and Citronella oil.

Another embodiment comprises cyclosporin A, Colatrope INC®, and Citronella oil.

Another embodiment comprises cyclosporin A, Crodacid B®, and Citronella oil.

Another embodiment comprises cyclosporin A, DeTrope CA-100®, and Citronella oil.

Another embodiment comprises cyclosporin A, Latol MTO®, and Citronella oil.

Another embodiment comprises cyclosporin A, Lumulse CC-33 K®, and Citronella oil.

Another embodiment comprises cyclosporin A, Mulls 2218®, and Citronella oil.

Another embodiment comprises cyclosporin A, OL-600®, and Citronella oil.

Another embodiment comprises cyclosporin A, OL-800®, and Citronella oil.

Another embodiment comprises cyclosporin A, R-910®, and Citronella oil.

Another embodiment comprises cyclosporin A, S-210®, and Citronella oil.

Another embodiment comprises cyclosporin A, Sandopan DTC Acid®, and Citronella oil.

Another embodiment comprises cyclosporin A, Sandopan LS 24 N®, and Citronella oil.

Another embodiment comprises cyclosporin A, Sandopan MA-18®, and Citronella oil.

Another embodiment comprises cyclosporin A, an Ethoxylated Alcohol, and Citronella oil.

Another embodiment comprises cyclosporin A, Adsee 799®, and Citronella oil.

Another embodiment comprises cyclosporin A, Adsee 799®, and Citronella oil.

Another embodiment comprises cyclosporin A, Alfonic 610-3.5®, and Citronella oil.

Another embodiment comprises cyclosporin A, Alfonic 810-2®, and Citronella oil.

Another embodiment comprises cyclosporin A, Alfonic 810-6®, and Citronella oil.

Another embodiment comprises cyclosporin A, Alfonic 1012-3®, and Citronella oil.

Another embodiment comprises cyclosporin A, Alfonic 1012-5®, and Citronella oil.

Another embodiment comprises cyclosporin A, Alfonic 1216CO-1.5®, and Citronella oil.

Another embodiment comprises cyclosporin A, Alfonic 1216CO-7®, and Citronella oil.

Another embodiment comprises cyclosporin A, Alfonic 1412-3®, and Citronella oil.

Another embodiment comprises cyclosporin A, Alfonic 1412-7®, and Citronella oil.

Another embodiment comprises cyclosporin A, Arlasolve 200®, and Citronella oil.

Another embodiment comprises cyclosporin A, Arlasolve 200 Liquid®, and Citronella oil.

Another embodiment comprises cyclosporin A, Armix 180-C®, and Citronella oil.

Another embodiment comprises cyclosporin A, Armix 183®, and Citronella oil.

Another embodiment comprises cyclosporin A, Armul 2404®, and Citronella oil.

Another embodiment comprises cyclosporin A, Armul 2404®, and Citronella oil.

Another embodiment comprises cyclosporin A, Atlas EMJ-C®, and Citronella oil.

Another embodiment comprises cyclosporin A, Atlas G-2109®, and Citronella oil.

Another embodiment comprises cyclosporin A, Atlas G-3886®, and Citronella oil.

Another embodiment comprises cyclosporin A, Atlas G-3890®, and Citronella oil.

Another embodiment comprises cyclosporin A, Bio Soft E-200®, and Citronella oil.

Another embodiment comprises cyclosporin A, Bio Soft E-300®, and Citronella oil.

Another embodiment comprises cyclosporin A, Bio Soft E-400®, and Citronella oil.

Another embodiment comprises cyclosporin A, Bio Soft EN 600®, and Citronella oil.

Another embodiment comprises cyclosporin A, Bio Soft TD-400®, and Citronella oil.

Another embodiment comprises cyclosporin A, Bio Soft TD-630®, and Citronella oil.

Another embodiment comprises cyclosporin A, Brij 30®, and Citronella oil.

Another embodiment comprises cyclosporin A, Brij 52®, and Citronella oil.

Another embodiment comprises cyclosporin A, Brij 56®, and Citronella oil.

Another embodiment comprises cyclosporin A, Brij 58®, and Citronella oil.

Another embodiment comprises cyclosporin A, Brij 72®, and Citronella oil.

Another embodiment comprises cyclosporin A, Brij 76®, and Citronella oil.

Another embodiment comprises cyclosporin A, Brij 78®, and Citronella oil.

Another embodiment comprises cyclosporin A, Brij 93®, and Citronella oil.

Another embodiment comprises cyclosporin A, Brij 97®, and Citronella oil.

Another embodiment comprises cyclosporin A, Brij 98®, and Citronella oil.

Another embodiment comprises cyclosporin A, Brij 700®, and Citronella oil.

Another embodiment comprises cyclosporin A, Brij 700 S®, and Citronelia oil.

Another embodiment comprises cyclosporin A, Brij 721®, and Citronella oil.

Another embodiment comprises cyclosporin A, Brij 721 S®, and Citronella oil.

Another embodiment comprises cyclosporin A, Burcoterge CDG®, and Citronella oil.

Another embodiment comprises cyclosporin A, Canasol AT 600®, and Citronella oil.

Another embodiment comprises cyclosporin A, Canasol AT 800®, and Citronella oil.

Another embodiment comprises cyclosporin A, Canasol AT 1200®, and Citronella oil.

Another embodiment comprises cyclosporin A, Canasol BJ 35®, and Citronella oil.

Another embodiment comprises cyclosporin A, Canasol BJ 36®, and Citronella oil.

Another embodiment comprises cyclosporin A, Canasol BJ 52®, and Citronella oil.

Another embodiment comprises cyclosporin A, Canasol BJ 58®, and Citronella oil.

Another embodiment comprises cyclosporin A, Canasol BJ 72®, and Citronella oil.

Another embodiment comprises cyclosporin A, Canasol BJ 78®, and Citronella oil.

Another embodiment comprises cyclosporin A, Canasol BJ 98®, and Citronella oil.

Another embodiment comprises cyclosporin A, Canasol BJ 307®, and Citronella oil.

Another embodiment comprises cyclosporin A, Cerfak 1400®, and Citronella oil.

Another embodiment comprises cyclosporin A, Cetomacrogol 1000 BP®, and Citronella oil.

Another embodiment comprises cyclosporin A, Chemonic C-2®, and Citronella oil.

Another embodiment comprises cyclosporin A, Chemonic C-10®, and Citronella oil.

Another embodiment comprises cyclosporin A, Chemonic C-20®, and Citronella oil.

Another embodiment comprises cyclosporin A, Chemonic CT-12®, and Citronella oil.

Another embodiment comprises cyclosporin A, Chemonic CT-20®, and Citronella oil.

Another embodiment comprises cyclosporin A, Chemonic CT-30®, and Citronella oil.

Another embodiment comprises cyclosporin A, Chemonic CT-55®, and Citronella oil.

Another embodiment comprises cyclosporin A, Chemonic G-7®, and Citronella oil.

Another embodiment comprises cyclosporin A, Chemonic G-26®, and Citronella oil.

Another embodiment comprises cyclosporin A, Chemonic L-4®, and Citronella oil.

Another embodiment comprises cyclosporin A, Chemonic L-7®, and Citronella oil.

Another embodiment comprises cyclosporin A, Chemonic L-12®, and Citronella oil.

Another embodiment comprises cyclosporin A, Chemonic L-23®, and Citronella oil.

Another embodiment comprises cyclosporin A, Chemonic O-2®, and Citronella oil.

Another embodiment comprises cyclosporin A, Chemonic O-5®, and Citronella oil.

Another embodiment comprises cyclosporin A, Chemonic O-10®, and Citronella oil.

Another embodiment comprises cyclosporin A, Chemonic O-20®, and Citronella oil.

Another embodiment comprises cyclosporin A, Chemonic S-2®, and Citronella oil.

Another embodiment comprises cyclosporin A, Chemonic S-10®, and Citronella oil.

Another embodiment comprises cyclosporin A, Chemonic S-20®, and Citronella oil.

Another embodiment comprises cyclosporin A, Colamulse FE®, and Citronella oil.

Another embodiment comprises cyclosporin A, Cremophor A 20®, and Citronella oil.

Another embodiment comprises cyclosporin A, Cremophor SA 2®, and Citronella oil.

Another embodiment comprises cyclosporin A, Dehydol 100®, and Citronella oil.

Another embodiment comprises cyclosporin A, Dehydol O-4®, and Citronella oil.

Another embodiment comprises cyclosporin A, Delonic C-18®, and Citronella oil.

Another embodiment comprises cyclosporin A, DeSonic 6T®, and Citronella oil.

Another embodiment comprises cyclosporin A, DeSonic 9D®, and Citronella oil.

Another embodiment comprises cyclosporin A, DeSonic 9T®, and Citronella oil.

Another embodiment comprises cyclosporin A, DeSonic 12D®, and Citronella oil.

Another embodiment comprises cyclosporin A, DeSonic 12T®, and Citronella oil.

Another embodiment comprises cyclosporin A, DeSonic 15T®, and Citronella oil.

Another embodiment comprises cyclosporin A, DeSonic TDA-9®, and Citronella oil.

Another embodiment comprises cyclosporin A, DeThox GLG-7®, and Citronella oil.

Another embodiment comprises cyclosporin A, DeThox GLG-26®, and Citronella oil.

Another embodiment comprises cyclosporin A, DeThox LA-4®, and Citronella oil.

Another embodiment comprises cyclosporin A, DeThox LA-23®, and Citronella oil.

Another embodiment comprises cyclosporin A, DeThox SA-80®, and Citronella oil.

Another embodiment comprises cyclosporin A, Disponil O5®, and Citronella oil.

Another embodiment comprises cyclosporin A, Eccoterge EO-41B®, and Citronella oil.

Another embodiment comprises cyclosporin A, Empilan KA2.5/90®, and Citronella oil.

Another embodiment comprises cyclosporin A, Empilan KA5/90®, and Citronella oil.

Another embodiment comprises cyclosporin A, Empilan KM-20®, and Citronella oil.

Another embodiment comprises cyclosporin A, Empilan KM-50®, and Citronella oil.

Another embodiment comprises cyclosporin A, Empilan L-23®, and Citronella oil.

Another embodiment comprises cyclosporin A, Ethylan 25-3®, and Citronella oil.

Another embodiment comprises cyclosporin A, Ethylan 1204®, and Citronella oil.

Another embodiment comprises cyclosporin A, Ethylan DA-4®, and Citronella oil.

Another embodiment comprises cyclosporin A, Ethylan LA-230®, and Citronella oil.

Another embodiment comprises cyclosporin A, Ethylan SN®, and Citronella oil.

Another embodiment comprises cyclosporin A, Ethylan TD-60®, and Citronella oil.

Another embodiment comprises cyclosporin A, Ethylan TD-100®, and Citronella oil.

Another embodiment comprises cyclosporin A, Ethylan TD-1407®, and Citronella oil.

Another embodiment comprises cyclosporin A, Eumulgin B1®, and Citronella oil.

Another embodiment comprises cyclosporin A, Eumulgin B2®, and Citronella oil.

Another embodiment comprises cyclosporin A, Eumulgin B3®, and Citronella oil.

Another embodiment comprises cyclosporin A, Eumulgin O-10®, and Citronella oil.

Another embodiment comprises cyclosporin A, Flo Mo 80/20®, and Citronella oil.

Another embodiment comprises cyclosporin A, Flo Mo Low Foam®, and Citronella oil.

Another embodiment comprises cyclosporin A, Forlan C-24®, and Citronella oil.

Another embodiment comprises cyclosporin A, Genapol 1454®, and Citronella oil.

Another embodiment comprises cyclosporin A, Genapol BA-020®, and Citronella oil.

Another embodiment comprises cyclosporin A, Genapol BA-040®, and Citronella oil.

Another embodiment comprises cyclosporin A, Genapol C-100®, and Citronella oil.

Another embodiment comprises cyclosporin A, Genapol DA 060®, and Citronella oil.

Another embodiment comprises cyclosporin A, Genapol HS 020®, and Citronella oil.

Another embodiment comprises cyclosporin A, Genapol HS 200®, and Citronella oil.

Another embodiment comprises cyclosporin A, Genapol ID-040®, and Citronella oil.

Another embodiment comprises cyclosporin A, Genapol ID-060®, and Citronella oil.

Another embodiment comprises cyclosporin A, Genapol ID-090®, and Citronella oil.

Another embodiment comprises cyclosporin A, Genapol LA 010®, and Citronella oil.

Another embodiment comprises cyclosporin A, Genapol LA 020®, and Citronella oil.

Another embodiment comprises cyclosporin A, Genapol LA 030®, and Citronella oil.

Another embodiment comprises cyclosporin A, Genapol LA 040®, and Citronella oil.

Another embodiment comprises cyclosporin A, Genapol LA 050®, and Citronella oil.

Another embodiment comprises cyclosporin A, Genapol LA 060®, and Citronella oil.

Another embodiment comprises cyclosporin A, Genapol LA 070®, and Citronella oil.

Another embodiment comprises cyclosporin A, Genapol LA 070S®, and Citronella oil.

Another embodiment comprises cyclosporin A, Genapol LA 230®, and Citronella oil.

Another embodiment comprises cyclosporin A, Genapol O 020®, and Citronella oil.

Another embodiment comprises cyclosporin A, Genapol O 050®, and Citronella oil.

Another embodiment comprises cyclosporin A, Genapol O 100®, and Citronella oil.

Another embodiment comprises cyclosporin A, Genapol O 200®, and Citronella oil.

Another embodiment comprises cyclosporin A, Genapol SA 030®, and Citronella oil.

Another embodiment comprises cyclosporin A, Genapol SA 120®, and Citronella oil.

Another embodiment comprises cyclosporin A, Genapol T-020®, and Citronella oil.

Another embodiment comprises cyclosporin A, Genapol UD-030®, and Citronella oil.

Another embodiment comprises cyclosporin A, Genapol UD-050®, and Citronella oil.

Another embodiment comprises cyclosporin A, Genapol UD-070®, and Citronella oil.

Another embodiment comprises cyclosporin A, Genapol UD-079®, and Citronella oil.

Another embodiment comprises cyclosporin A, Genapol UD-080®, and Citronella oil.

Another embodiment comprises cyclosporin A, Genapol UD-110®, and Citronella oil.

Another embodiment comprises cyclosporin A, Genapol X 030®, and Citronella oil.

Another embodiment comprises cyclosporin A, Genapol X 050®, and Citronella oil.

Another embodiment comprises cyclosporin A, Genapol X 060®, and Citronella oil.

Another embodiment comprises cyclosporin A, Genapol X 070®, and Citronella oil.

Another embodiment comprises cyclosporin A, Genapol X 080®, and Citronella oil.

Another embodiment comprises cyclosporin A, Genapol X 100®, and Citronella oil.

Another embodiment comprises cyclosporin A, Genapol X 159®, and Citronella oil.

Another embodiment comprises cyclosporin A, Generol 122 E5®, and Citronella oil.

Another embodiment comprises cyclosporin A, Generol 122 E25®, and Citronella oil.

Another embodiment comprises cyclosporin A, Hostacerin T-3®, and Citronella oil.

Another embodiment comprises cyclosporin A, Iconol LF 110®, and Citronella oil.

Another embodiment comprises cyclosporin A, Incropol CS-20®, and Citronella oil.

Another embodiment comprises cyclosporin A, Lexemul CS-20®, and Citronella oil.

Another embodiment comprises cyclosporin A, Liponic EG-1®, and Citronella oil.

Another embodiment comprises cyclosporin A, Lipowax D®, and Citronella oil.

Another embodiment comprises cyclosporin A, Lipowax G®, and Citronella oil.

Another embodiment comprises cyclosporin A, Lipowax NI®, and Citronella oil.

Another embodiment comprises cyclosporin A, Lipowax P®, and Citronella oil.

Another embodiment comprises cyclosporin A, Lipowax P-31®, and Citronella oil.

Another embodiment comprises cyclosporin A, Lipowax PR®, and Citronella oil.

Another embodiment comprises cyclosporin A, Lumulse CS-20®, and Citronella oil.

Another embodiment comprises cyclosporin A, Macol CSA-20®, and Citronella oil.

Another embodiment comprises cyclosporin A, Marlox B 24/50®, and Citronella oil.

Another embodiment comprises cyclosporin A, Mazawet 77®, and Citronella oil.

Another embodiment comprises cyclosporin A, Norfox 1713®, and Citronella oil.

Another embodiment comprises cyclosporin A, Norfox 2579®, and Citronella oil.

Another embodiment comprises cyclosporin A, Norfox Lo Foam®, and Citronella oil.

Another embodiment comprises cyclosporin A, Promulgen D®, and Citronella oil.

Another embodiment comprises cyclosporin A, Promulgen G®, and Citronella oil.

Another embodiment comprises cyclosporin A, Renex 30®, and Citronella oil.

Another embodiment comprises cyclosporin A, Renex 36®, and Citronella oil.

Another embodiment comprises cyclosporin A, Rhodasurf A 24®, and Citronella oil.

Another embodiment comprises cyclosporin A, Rhodasurf AAE-10®, and Citronella oil.

Another embodiment comprises cyclosporin A, Rhodasurf BEH-25®, and Citronella oil.

Another embodiment comprises cyclosporin A, Rhodasurf BEH-40®, and Citronella oil.

Another embodiment comprises cyclosporin A, Rhodasurf DA 530®, and Citronella oil.

Another embodiment comprises cyclosporin A, Rhodasurf DA 630®, and Citronella oil.

Another embodiment comprises cyclosporin A, Rhodasurf DA 639®, and Citronella oil.

Another embodiment comprises cyclosporin A, Rhodasurf LAN-23®, and Citronella oil.

Another embodiment comprises cyclosporin A, Rhodasurf ON-870®, and Citronella oil.

Another embodiment comprises cyclosporin A, Rhodasurf ON-877®, and Citronella oil.

Another embodiment comprises cyclosporin A, Rhodasurf TB-970 FLK®, and Citronella oil.

Another embodiment comprises cyclosporin A, Ritacet-20®, and Citronella oil.

Another embodiment comprises cyclosporin A, Ritachol 1000®, and Citronella oil.

Another embodiment comprises cyclosporin A, Ritachol 2000®, and Citronella oil.

Another embodiment comprises cyclosporin A, Ritachol 5000®, and Citronella oil.

Another embodiment comprises cyclosporin A, Ritox 35®, and Citronella oil.

Another embodiment comprises cyclosporin A, Surfonic DA-4®, and Citronella oil.

Another embodiment comprises cyclosporin A, Surfonic DA-6®, and Citronella oil.

Another embodiment comprises cyclosporin A, Surfonic L46-7®, and Citronella oil.

Another embodiment comprises cyclosporin A, Surfonic POA®, and Citronella oil.

Another embodiment comprises cyclosporin A, Synthrapol KB®, and Citronella oil.

Another embodiment comprises cyclosporin A, Teginacid®, and Citronella oil.

Another embodiment comprises cyclosporin A, Teginacid C®, and Citronella oil.

Another embodiment comprises cyclosporin A, Tegotens EC 11®, and Citronella oil.

Another embodiment comprises cyclosporin A, Tinegal NA®, and Citronella oil.

Another embodiment comprises cyclosporin A, Tomadol 400®, and Citronella oil.

Another embodiment comprises cyclosporin A, Tomadol 600®, and Citronella oil.

Another embodiment comprises cyclosporin A, Tomadol 900®, and Citronella oil.

Another embodiment comprises cyclosporin A, Uniperol O®, and Citronella oil.

Another embodiment comprises cyclosporin A, Witconol SN Series®, and Citronella oil.

Another embodiment comprises cyclosporin A, an Ethoxylated Alkylphenol, and Citronella oil.

Another embodiment comprises cyclosporin A, Antarox LF-222®, and Citronella oil.

Another embodiment comprises cyclosporin A, Atlox 775®, and Citronella oil.

Another embodiment comprises cyclosporin A, Caloxylate N-9®, and Citronella oil.

Another embodiment comprises cyclosporin A, Canasol NF-1000®, and Citronella oil.

Another embodiment comprises cyclosporin A, Canasol NF-3000®, and Citronella oil.

Another embodiment comprises cyclosporin A, Canasol NF-3070®, and Citronella oil.

Another embodiment comprises cyclosporin A, Canasol OF 1670®, and Citronella oil.

Another embodiment comprises cyclosporin A, Canasol OF 2570®, and Citronella oil.

Another embodiment comprises cyclosporin A, Canasol OF 4070®, and Citronella oil.

Another embodiment comprises cyclosporin A, Chemax DNP-8®, and Citronella oil.

Another embodiment comprises cyclosporin A, Chemax DNP-18®, and Citronella oil.

Another embodiment comprises cyclosporin A, Chemax DNP-150/50®, and Citronella oil.

Another embodiment comprises cyclosporin A, DeSonic 1.5N®, and Citronella oil.

Another embodiment comprises cyclosporin A, DeSonic 4N®, and Citronella oil.

Another embodiment comprises cyclosporin A, DeSonic 5N®, and Citronella oil.

Another embodiment comprises cyclosporin A, DeSonic 6D®, and Citronella oil.

Another embodiment comprises cyclosporin A, DeSonic 6N®, and Citronella oil.

Another embodiment comprises cyclosporin A, DeSonic 7N®, and Citronella oil.

Another embodiment comprises cyclosporin A, DeSonic 9N®, and Citronella oil.

Another embodiment comprises cyclosporin A, DeSonic 10D®, and Citronella oil.

Another embodiment comprises cyclosporin A, DeSonic 11N®, and Citronella oil.

Another embodiment comprises cyclosporin A, DeSonic 12N®, and Citronella oil.

Another embodiment comprises cyclosporin A, DeSonic 15N®, and Citronella oil.

Another embodiment comprises cyclosporin A, DeSonic 20N®, and Citronella oil.

Another embodiment comprises cyclosporin A, Eccoscour RC®, and Citronella oil.

Another embodiment comprises cyclosporin A, Eccoterge EO-100®, and Citronella oil.

Another embodiment comprises cyclosporin A, Emulsifier 632/90%®, and Citronella oil.

Another embodiment comprises cyclosporin A, Geronol AG-821®, and Citronella oil.

Another embodiment comprises cyclosporin A, Gradonic N-95®, and Citronella oil.

Another embodiment comprises cyclosporin A, Hetoxide NP-4®, and Citronella oil.

Another embodiment comprises cyclosporin A, Hetoxide NP-30®, and Citronella oil.

Another embodiment comprises cyclosporin A, Hostapal N-100®, and Citronella oil.

Another embodiment comprises cyclosporin A, Hostapal N-110®, and Citronella oil.

Another embodiment comprises cyclosporin A, Igepal CTA-639W®, and Citronella oil.

Another embodiment comprises cyclosporin A, Igepal DAP-9®, and Citronella oil.

Another embodiment comprises cyclosporin A, Igepal OD-410®, and Citronella oil.

Another embodiment comprises cyclosporin A, Igepal SS-837®, and Citronella oil.

Another embodiment comprises cyclosporin A, Lipocol NP-9 USP®, and Citronella oil.

Another embodiment comprises cyclosporin A, Macol DNP-10®, and Citronella oil.

Another embodiment comprises cyclosporin A, Marlophen NP 5®, and Citronella oil.

Another embodiment comprises cyclosporin A, Marlophen P 1®, and Citronella oil.

Another embodiment comprises cyclosporin A, Surfonic NB®, and Citronella oil.

Another embodiment comprises cyclosporin A, Surfonic OPB-307®, and Citronella oil.

Another embodiment comprises cyclosporin A, Surfonic OPB-407®, and Citronella oil.

Another embodiment comprises cyclosporin A, Syn Fac 334®, and Citronella oil.

Another embodiment comprises cyclosporin A, Syn Fac-8216®, and Citronella oil.

Another embodiment comprises cyclosporin A, Triton N-57®, and Citronella oil.

Another embodiment comprises cyclosporin A, Trycol 6956®, and Citronella oil.

Another embodiment comprises cyclosporin A, Trycol 6961®, and Citronella oil.

Another embodiment comprises cyclosporin A, Trycol 6964®, and Citronella oil.

Another embodiment comprises cyclosporin A, Trycol 6969®, and Citronella oil.

Another embodiment comprises cyclosporin A, Trycol 6974®, and Citronella oil.

Another embodiment comprises cyclosporin A, Witbreak DRB-127®, and Citronella oil.

Another embodiment comprises cyclosporin A, Witbreak DRB-127®, and Citronella oil.

Another embodiment comprises cyclosporin A, Witconol NP Series®, and Citronella oil.

Another embodiment comprises cyclosporin A, an Ethoxylated Aryl Phenol, and Citronella oil.

Another embodiment comprises cyclosporin A, Soprophor BSU®, and Citronella oil.

Another embodiment comprises cyclosporin A, Soprophor CY/8®, and Citronella oil.

Another embodiment comprises cyclosporin-A, Soprophor S/25®, and Citronella oil.

Another embodiment comprises cyclosporin A, Witconol NIO®, and Citronella oil.

Another embodiment comprises cyclosporin A, Witconol NIW®, and Citronella oil.

Another embodiment comprises cyclosporin A, Witconol S-100®, and Citronella oil.

Another embodiment comprises cyclosporin A, an Ethoxylated Fatty Acid, and Citronella oil.

Another embodiment comprises cyclosporin A, Aldo PGHMS®, and Citronella oil.

Another embodiment comprises cyclosporin A, Alkamuls TO-15/HR®, and Citronella oil.

Another embodiment comprises cyclosporin A, Armotan AL-69-66®, and Citronella oil.

Another embodiment comprises cyclosporin A, Cerasynt 840®, and Citronella oil.

Another embodiment comprises cyclosporin A, Cerasynt 945®, and Citronella oil.

Another embodiment comprises cyclosporin A, Crystal Inhibitor No. 5®, and Citronella oil.

Another embodiment comprises cyclosporin A, DeThox Acid L-9®, and Citronella oil.

Another embodiment comprises cyclosporin A, DeThox Acid S-8®, and Citronella oil.

Another embodiment comprises cyclosporin A, Ethofat 242/25®, and Citronella oil.

Another embodiment comprises cyclosporin A, Hydropalat 65®, and Citronella oil.

Another embodiment comprises cyclosporin A, Lipo EGMS®, and Citronella oil.

Another embodiment comprises cyclosporin A, Lipopeg 2 DL®, and Citronella oil.

Another embodiment comprises cyclosporin A, Lipopeg 4 DL®, and Citronella oil.

Another embodiment comprises cyclosporin A, Lipopeg 4-L®, and Citronella oil.

Another embodiment comprises cyclosporin A, Lipopeg 39-S®, and Citronella oil.

Another embodiment comprises cyclosporin A, Lipopeg 4-S®, and Citronella oil.

Another embodiment comprises cyclosporin A, Lipopeg 10-S®, and Citronella oil.

Another embodiment comprises cyclosporin A, Lipopeg 100-S®, and Citronella oil.

Another embodiment comprises cyclosporin A, Lipopeg 6000 DS®, and Citronella oil.

Another embodiment comprises cyclosporin A, Lumulse 40-L®, and Citronella oil.

Another embodiment comprises cyclosporin A, Lumulse 40-S®, and Citronella oil.

Another embodiment comprises cyclosporin A, Lumulse 42-L®, and Citronella oil.

Another embodiment comprises cyclosporin A, Lumulse 42-S®, and Citronella oil.

Another embodiment comprises cyclosporin A, Lumulse 100-S®, and Citronella oil.

Another embodiment comprises cyclosporin A, Lumulse 602-S®, and Citronella oil.

Another embodiment comprises cyclosporin A, Magrabar PGE-20-0®, and Citronella oil.

Another embodiment comprises cyclosporin A, Magrabar PGE-20L®, and Citronella oil.

Another embodiment comprises cyclosporin A, Magrabar PGE-20T®, and Citronelia oil.

Another embodiment comprises cyclosporin A, Magrabar PGE-22-0®, and Citronella oil.

Another embodiment comprises cyclosporin A, Magrabar PGE-22L®, and Citronella oil.

Another embodiment comprises cyclosporin A, Magrabar PGE-22T®, and Citronella oil.

Another embodiment comprises cyclosporin A, Magrabar PGE-40-0®, and Citronella oil.

Another embodiment comprises cyclosporin A, Magrabar PGE-40L®, and Citronella oil.

Another embodiment comprises cyclosporin A, Magrabar PGE-40T®, and Citronella oil.

Another embodiment comprises cyclosporin A, Magrabar PGE-42-0®, and Citronella oil.

Another embodiment comprises cyclosporin A, Magrabar PGE-42L®, and Citronella oil.

Another embodiment comprises cyclosporin A, Magrabar PGE-42T®, and Citronella oil.

Another embodiment comprises cyclosporin A, Magrabar PGE-60-0®, and Citronella oil.

Another embodiment comprises cyclosporin A, Magrabar PGE-60L®, and Citronella oil.

Another embodiment comprises cyclosporin A, Magrabar PGE-60T®, and Citronella oil.

Another embodiment comprises cyclosporin A, Magrabar PGE-62-0®, and Citronella oil.

Another embodiment comprises cyclosporin A, Magrabar PGE-62L®, and Citronella oil.

Another embodiment comprises cyclosporin A, Magrabar PGE-62T®, and Citronella oil.

Another embodiment comprises cyclosporin A, Mapeg S-40K®, and Citronella oil.

Another embodiment comprises cyclosporin A, Marlowet OTS®, and Citronella oil.

Another embodiment comprises cyclosporin A, Naturechem PGR®, and Citronella oil.

Another embodiment comprises cyclosporin A, PG No. 4®, and Citronella oil.

Another embodiment comprises cyclosporin A, Renex 20®, and Citronella oil.

Another embodiment comprises cyclosporin A, Ritox 52®, and Citronella oil.

Another embodiment comprises cyclosporin A, Ritox 53®, and Citronella oil.

Another embodiment comprises cyclosporin A, Ritox 59®, and Citronella oil.

Another embodiment comprises cyclosporin A, Surax 8916/A®, and Citronella oil.

Another embodiment comprises cyclosporin A, Tego Acid S 40 P®, and Citronella oil.

Another embodiment comprises cyclosporin A, Tego Acid S 100 P®, and Citronella oil.

Another embodiment comprises cyclosporin A, Tween 20®, and Citronella oil.

Another embodiment comprises cyclosporin A, Volpo 131®, and Citronella oil.

Another embodiment comprises cyclosporin A, an Ethoxylated Fatty Ester or Oil, and Citronella oil.

Another embodiment comprises cyclosporin A, Acconon 6-C10®, and Citronella oil.

Another embodiment comprises cyclosporin A, Acconon CC-6®, and Citronella oil.

Another embodiment comprises cyclosporin A, Acconon CO-7®, and Citronella oil.

Another embodiment comprises cyclosporin A, Aldosperse 40/60 FG®, and Citronella oil.

Another embodiment comprises cyclosporin A, Aldosperse ML-23®, and Citronella oil.

Another embodiment comprises cyclosporin A, Aldosperse MS-20 FG®, and Citronella oil.

Another embodiment comprises cyclosporin A, Alkamuls EL-620®, and Citronella oil.

Another embodiment comprises cyclosporin A, Alkamuls EL-719®, and Citronella oil.

Another embodiment comprises cyclosporin A, Alkamuls EL-985®, and Citronella oil.

Another embodiment comprises cyclosporin A, Arlatone G®, and Citronella oil.

Another embodiment comprises cyclosporin A, Arlatone T®, and Citronella oil.

Another embodiment comprises cyclosporin A, Atlas G-1045A®, and Citronella oil.

Another embodiment comprises cyclosporin A, Atlas G-1086®, and Citronella oil.

Another embodiment comprises cyclosporin A, Atlas G-1087®, and Citronella oil.

Another embodiment comprises cyclosporin A, Atlas G-1089®, and Citronella oil.

Another embodiment comprises cyclosporin A, Atlas G-1096®, and Citronella oil.

Another embodiment comprises cyclosporin A, Atlas G-1292®, and Citronella oil.

Another embodiment comprises cyclosporin A, Atlas G-1293®, and Citronella oil.

Another embodiment comprises cyclosporin A, Atlas G-1300®, and Citronella oil.

Another embodiment comprises cyclosporin A, Atlas G-7076®, and Citronella oil.

Another embodiment comprises cyclosporin A, Capmul EMG®, and Citronella oil.

Another embodiment comprises cyclosporin A, Chemonic CC-40®, and Citronella oil.

Another embodiment comprises cyclosporin A, Chemonic LI-3®, and Citronella oil.

Another embodiment comprises cyclosporin A, Chemonic LI-7®, and Citronella oil.

Another embodiment comprises cyclosporin A, Cirrasol GM®, and Citronella oil.

Another embodiment comprises cyclosporin A, Cremophor CO 40®, and Citronella oil.

Another embodiment comprises cyclosporin A, Cremophor CO 410®, and Citronella oil.

Another embodiment comprises cyclosporin A, Cremophor EL®, and Citronella oil.

Another embodiment comprises cyclosporin A, Cremophor GC7®, and Citronella oil.

Another embodiment comprises cyclosporin A, Cremophor RH-40®, and Citronella oil.

Another embodiment comprises cyclosporin A, Crovol A-40®, and Citronella oil.

Another embodiment comprises cyclosporin A, Crovol A-70®, and Citronella oil.

Another embodiment comprises cyclosporin A, Crovol M-70®, and Citronella oil.

Another embodiment comprises cyclosporin A, Crovol PK-70®, and Citronella oil.

Another embodiment comprises cyclosporin A, Cutina E-24®, and Citronella oil.

Another embodiment comprises cyclosporin A, Dacospin 12-R®, and Citronella oil.

Another embodiment comprises cyclosporin A, Dehymuls HRE-7®, and Citronella oil.

Another embodiment comprises cyclosporin A, DeSonic 30C®, and Citronella oil.

Another embodiment comprises cyclosporin A, DeSonic 36C®, and Citronella oil.

Another embodiment comprises cyclosporin A, DeSonic 40C®, and Citronella oil.

Another embodiment comprises cyclosporin A, Durfax 60®, and Citronella oil.

Another embodiment comprises cyclosporin A, Durfax 65®, and Citronella oil.

Another embodiment comprises cyclosporin A, Durfax 80®, and Citronella oil.

Another embodiment comprises cyclosporin A, Durfax EOM®, and Citronella oil.

Another embodiment comprises cyclosporin A, Eccoterge NF-2®, and Citronella oil.

Another embodiment comprises cyclosporin A, Emulpon CO-360®, and Citronella oil.

Another embodiment comprises cyclosporin A, Emulpon CO-550®, and Citronella oil.

Another embodiment comprises cyclosporin A, Emulsogen EL®, and Citronella oil.

Another embodiment comprises cyclosporin A, Emulsogen HCO 040®, and Citronella oil.

Another embodiment comprises cyclosporin A, Emulsogen HCO 060®, and Citronella oil.

Another embodiment comprises cyclosporin A, Emulsynt 1055®, and Citronella oil.

Another embodiment comprises cyclosporin A, Ethox 3095®, and Citronella oil.

Another embodiment comprises cyclosporin A, Eumulgin RO-40®, and Citronella oil.

Another embodiment comprises cyclosporin A, Genapol G-260®, and Citronella oil.

Another embodiment comprises cyclosporin A, Glycosperse L-20®, and Citronella oil.

Another embodiment comprises cyclosporin A, Glycosperse O-5®, and Citronella oil.

Another embodiment comprises cyclosporin A, Glycosperse O-20®, and Citronella oil.

Another embodiment comprises cyclosporin A, Glycosperse O-20 FG®, and Citronella oil.

Another embodiment comprises cyclosporin A, Glycosperse S-20®, and Citronella oil.

Another embodiment comprises cyclosporin A, Glycosperse S-20 FG®, and Citronella oil.

Another embodiment comprises cyclosporin A, Glycosperse TS-20®, and Citronella oil.

Another embodiment comprises cyclosporin A, Glycosperse TS-20 FG®, and Citronella oil.

Another embodiment comprises cyclosporin A, Hetan SL®, and Citronella oil.

Another embodiment comprises cyclosporin A, Hetan SO®, and Citronella oil.

Another embodiment comprises cyclosporin A, Hetan SS®, and Citronella oil.

Another embodiment comprises cyclosporin A, Hetoxide C-2®, and Citronella oil.

Another embodiment comprises cyclosporin A, Hetoxide C-9®, and Citronella oil.

Another embodiment comprises cyclosporin A, Hetoxide C-15®, and Citronella oil.

Another embodiment comprises cyclosporin A, Hetoxide C-25®, and Citronella oil.

Another embodiment comprises cyclosporin A, Hetoxide C-40®, and Citronella oil.

Another embodiment comprises cyclosporin A, Hetoxide C-200®, and Citronella oil.

Another embodiment comprises cyclosporin A, Hetoxide C-200-50%®, and Citronella oil.

Another embodiment comprises cyclosporin A, Hetoxide GC-30®, and Citronella oil.

Another embodiment comprises cyclosporin A, Hetoxide HC-60®, and Citronella oil.

Another embodiment comprises cyclosporin A, Ice No. 2®, and Citronella oil.

Another embodiment comprises cyclosporin A, Incrocas 30/40®, and Citronella oil.

Another embodiment comprises cyclosporin A, Lexol EC®, and Citronella oil.

Another embodiment comprises cyclosporin A, Lexol EO®, and Citronella oil.

Another embodiment comprises cyclosporin A, Lipocol HCO-40®, and Citronella oil.

Another embodiment comprises cyclosporin A, Lipocol HCO-60®, and Citronella oil.

Another embodiment comprises cyclosporin A, Lipocol O-3 Special®, and Citronella oil.

Another embodiment comprises cyclosporin A, Lipopeg 2-L®, and Citronella oil.

Another embodiment comprises cyclosporin A, Lipopeg 4-DO®, and Citronella oil.

Another embodiment comprises cyclosporin A, Lipopeg 4-DS®, and Citronella oil.

Another embodiment comprises cyclosporin A, Lipovol GTB®, and Citronella oil.

Another embodiment comprises cyclosporin A, Lonzest SML-20®, and Citronella oil.

Another embodiment comprises cyclosporin A, Lonzest SMO-20®, and Citronella oil.

Another embodiment comprises cyclosporin A, Lonzest SMS-20®, and Citronella oil.

Another embodiment comprises cyclosporin A, Lonzest STO-20®, and Citronella oil.

Another embodiment comprises cyclosporin A, Lonzest STS-20®, and Citronella oil.

Another embodiment comprises cyclosporin A, Lumulse GR-40®, and Citronella oil.

Another embodiment comprises cyclosporin A, Lumulse GRH-40®, and Citronella oil.

Another embodiment comprises cyclosporin A, Lumulse POE (7) GML®, and Citronella oil.

Another embodiment comprises cyclosporin A, Lumulse POE (12) Glyc®, and Citronella oil.

Another embodiment comprises cyclosporin A, Lumulse POE (40) MS KP®, and Citronella oil.

Another embodiment comprises cyclosporin A, Marlowet 4750®, and Citronella oil.

Another embodiment comprises cyclosporin A, Marlowet LVS®, and Citronella oil.

Another embodiment comprises cyclosporin A, Marlowet R 11®, and Citronella oil.

Another embodiment comprises cyclosporin A, Marlowet R 40®, and Citronella oil.

Another embodiment comprises cyclosporin A, Mazol 80 MGK®, and Citronella oil.

Another embodiment comprises cyclosporin A, Nonionic Emulsifier T-9®, and Citronella oil.

Another embodiment comprises cyclosporin A, Oronal LCG®, and Citronella oil.

Another embodiment comprises cyclosporin A, Polyderm PPI-CO-200®, and Citronella oil.

Another embodiment comprises cyclosporin A, Polyderm PPI-CO-40®, and Citronella oil.

Another embodiment comprises cyclosporin A, Rewoderm LI 520-70®, and Citronella oil.

Another embodiment comprises cyclosporin A, Ritapeg 150 DS®, and Citronella oil.

Another embodiment comprises cyclosporin A, Softigen 767®, and Citronella oil.

Another embodiment comprises cyclosporin A, Surfactol 318®, and Citronella oil.

Another embodiment comprises cyclosporin A, Surfactol 365®, and Citronella oil.

Another embodiment comprises cyclosporin A, Syn Lube 107®, and Citronella oil.

Another embodiment comprises cyclosporin A, Syn Lube 728®, and Citronella oil.

Another embodiment comprises cyclosporin A, Syn Lube 1632H®, and Citronella oil.

Another embodiment comprises cyclosporin A, Syn Lube 6277-A®, and Citronella oil.

Another embodiment comprises cyclosporin A, T-Det C-20®, and Citronella oil.

Another embodiment comprises cyclosporin A, T-Det C-40®, and Citronella oil.

Another embodiment comprises cyclosporin A, Tally 100 Plus®, and Citronella oil.

Another embodiment comprises cyclosporin A, Uniperol EL®, and Citronella oil.

Another embodiment comprises cyclosporin A, a Fatty Ester, and Citronella oil.

Another embodiment comprises cyclosporin A, Actralube-Syn 147®, and Citronella oil.

Another embodiment comprises cyclosporin A, Atlas G-1556®, and Citronella oil.

Another embodiment comprises cyclosporin A, Atlas G-1564®, and Citronella oil.

Another embodiment comprises cyclosporin A, Atlasol Base Oil S®, and Citronella oil.

Another embodiment comprises cyclosporin A, Base ML®, and Citronella oil.

Another embodiment comprises cyclosporin A, Base MT®, and Citronella oil.

Another embodiment comprises cyclosporin A, Cerasynt 303®, and Citronella oil.

Another embodiment comprises cyclosporin A, Dermol 1012®, and Citronella oil.

Another embodiment comprises cyclosporin A, Kemester 4000®, and Citronella oil.

Another embodiment comprises cyclosporin A, Lactipol S®, and Citronella oil.

Another embodiment comprises cyclosporin A, Magrabar PGO®, and Citronella oil.

Another embodiment comprises cyclosporin A, Mayco Base BFO®, and Citronella oil.

Another embodiment comprises cyclosporin A, Methyl Linoleate®, and Citronella oil.

Another embodiment comprises cyclosporin A, Pationic 122A®, and Citronella oil.

Another embodiment comprises cyclosporin A, Pationic 138C®, and Citronella oil.

Another embodiment comprises cyclosporin A, Pationic CSL®, and Citronella oil.

Another embodiment comprises cyclosporin A, Pationic ISL®, and Citronella oil.

Another embodiment comprises cyclosporin A, Pationic SBL®, and Citronella oil.

Another embodiment comprises cyclosporin A, Pationic SSL®, and Citronella oil.

Another embodiment comprises cyclosporin A, Ritasol®, and Citronella oil.

Another embodiment comprises cyclosporin A, Tego Alkanol CS 20®, and Citronella oil.

Another embodiment comprises cyclosporin A, Tego Alkanol L23 P®, and Citronella oil.

Another embodiment comprises cyclosporin A, Tego Alkanol S2®, and Citronella oil.

Another embodiment comprises cyclosporin A, Tego Alkanol S20 P®, and Citronella oil.

Another embodiment comprises cyclosporin A, Triemulsifier 600 MS®, and Citronella oil.

Another embodiment comprises cyclosporin A, a Glycerol Esters, and Citronella oil.

Another embodiment comprises cyclosporin A, Agro #9 Wint SBO®, and Citronella oil.

Another embodiment comprises cyclosporin A, Ahcovel Base 700®, and Citronella oil.

Another embodiment comprises cyclosporin A, Aldo HMS FG®, and Citronella oil.

Another embodiment comprises cyclosporin A, Aldo MLD®, and Citronella oil.

Another embodiment comprises cyclosporin A, Aldo MLD FG®, and Citronella oil.

Another embodiment comprises cyclosporin A, Aldo MO FG®, and Citronella oil.

Another embodiment comprises cyclosporin A, Aldo MS®, and Citronella oil.

Another embodiment comprises cyclosporin A, Aldo MS FG®, and Citronella oil.

Another embodiment comprises cyclosporin A, Aldo MS LG FG®, and Citronella oil.

Another embodiment comprises cyclosporin A, Aldo MSD®, and Citronella oil.

Another embodiment comprises cyclosporin A, Aldo MSD FG®, and Citronella oil.

Another embodiment comprises cyclosporin A, Aldosperse O-20 FG®, and Citronella oil.

Another embodiment comprises cyclosporin A, Aldosperse TS-20 FG®, and Citronella oil.

Another embodiment comprises cyclosporin A, Aldosperse TS-40 FG®, and Citronella oil.

Another embodiment comprises cyclosporin A, Arlacel 165®, and Citronella oil.

Another embodiment comprises cyclosporin A, Arlacel 186®, and Citronella oil.

Another embodiment comprises cyclosporin A, Capmul GMO®, and Citronella oil.

Another embodiment comprises cyclosporin A, Capmul GMS®, and Citronella oil.

Another embodiment comprises cyclosporin A, Caprol 3GO®, and Citronella oil.

Another embodiment comprises cyclosporin A, Caprol 3GVS®, and Citronella oil.

Another embodiment comprises cyclosporin A, Caprol 6G2S®, and Citronella oil.

Another embodiment comprises cyclosporin A, Caprol 10G40®, and Citronella oil.

Another embodiment comprises cyclosporin A, Caprol 10G100®, and Citronella oil.

Another embodiment comprises cyclosporin A, Caprol ET®, and Citronella oil.

Another embodiment comprises cyclosporin A, Caprol PGE860®, and Citronella oil.

Another embodiment comprises cyclosporin A, Cerasynt 945®, and Citronella oil.

Another embodiment comprises cyclosporin A, Cerasynt GMS®, and Citronella oil.

Another embodiment comprises cyclosporin A, Cerasynt Q®, and Citronella oil.

Another embodiment comprises cyclosporin A, Cerasynt SD®, and Citronella oil.

Another embodiment comprises cyclosporin A, Cerasynt WM®, and Citronella oil.

Another embodiment comprises cyclosporin A, Chemsperse 14®, and Citronella oil.

Another embodiment comprises cyclosporin A, Cremophor GO-32®, and Citronella oil.

Another embodiment comprises cyclosporin A, Cremophor GS11®, and Citronella oil.

Another embodiment comprises cyclosporin A, Cremophor GS-32®, and Citronella oil.

Another embodiment comprises cyclosporin A, Cutina KD-16®, and Citronella oil.

Another embodiment comprises cyclosporin A, Dehymuls PGPH®, and Citronella oil.

Another embodiment comprises cyclosporin A, Dermol DGDIS®, and Citronella oil.

Another embodiment comprises cyclosporin A, Dermol DGMIS®, and Citronella oil.

Another embodiment comprises cyclosporin A, Dermol G-76®, and Citronella oil.

Another embodiment comprises cyclosporin A, Dermol G-7DI®, and Citronella oil.

Another embodiment comprises cyclosporin A, Dermol NGDI®, and Citronella oil.

Another embodiment comprises cyclosporin A, Dermolan GLH®, and Citronella oil.

Another embodiment comprises cyclosporin A, Drewmulse GMO®, and Citronella oil.

Another embodiment comprises cyclosporin A, Drewpol 3-5-M®, and Citronella oil.

Another embodiment comprises cyclosporin A, Durlac 100 W®, and Citronella oil.

Another embodiment comprises cyclosporin A, Dur-Lo®, and Citronella oil.

Another embodiment comprises cyclosporin A, Dynasan 118®, and Citronella oil.

Another embodiment comprises cyclosporin A, EC-25®, and Citronella oil.

Another embodiment comprises cyclosporin A, EM 40®, and Citronella oil.

Another embodiment comprises cyclosporin A, Emerest 2400®, and Citronella oil.

Another embodiment comprises cyclosporin A, Emerest 2452®, and Citronella oil.

Another embodiment comprises cyclosporin A, Empilan G-26®, and Citronella oil.

Another embodiment comprises cyclosporin A, Genapol TSM®, and Citronella oil.

Another embodiment comprises cyclosporin A, Hostacerin DGI®, and Citronella oil.

Another embodiment comprises cyclosporin A, Hostacerin DGL®, and Citronella oil.

Another embodiment comprises cyclosporin A, Hostacerin DGMS®, and Citronella oil.

Another embodiment comprises cyclosporin A, Hostacerin DGSB®, and Citronella oil.

Another embodiment comprises cyclosporin A, Ice No. 2®, and Citronella oil.

Another embodiment comprises cyclosporin A, Imwitor 742®, and Citronella oil.

Another embodiment comprises cyclosporin A, Imwitor 780 K®, and Citronella oil.

Another embodiment comprises cyclosporin A, Imwitor 960 Flakes®, and Citronella oil.

Another embodiment comprises cyclosporin A, Isolan GI 34®, and Citronella oil.

Another embodiment comprises cyclosporin A, Isolan GO 33®, and Citronella oil.

Another embodiment comprises cyclosporin A, Kemester 1000®, and Citronella oil.

Another embodiment comprises cyclosporin A, Kemester 2000®, and Citronella oil.

Another embodiment comprises cyclosporin A, Kemester 2000®, and Citronella oil.

Another embodiment comprises cyclosporin A, Kemester 6000SE®, and Citronella oil.

Another embodiment comprises cyclosporin A, Lamecreme DGE 18®, and Citronella oil.

Another embodiment comprises cyclosporin A, Lexemul 515®, and Citronella oil.

Another embodiment comprises cyclosporin A, Lexemul 561®, and Citronella oil.

Another embodiment comprises cyclosporin A, Lexemul AR®, and Citronella oil.

Another embodiment comprises cyclosporin A, Lexemul AS®, and Citronella oil.

Another embodiment comprises cyclosporin A, Lexemul GDL®, and Citronella oil.

Another embodiment comprises cyclosporin A, Lexemul T®, and Citronella oil.

Another embodiment comprises cyclosporin A, Lipomulse 165®, and Citronella oil.

Another embodiment comprises cyclosporin A, Lumulse GML K®, and Citronella oil.

Another embodiment comprises cyclosporin A, Lumulse GMO K®, and Citronella oil.

Another embodiment comprises cyclosporin A, Lumulse GMR K®, and Citronella oil.

Another embodiment comprises cyclosporin A, Lumulse GMT K®, and Citronella oil.

Another embodiment comprises cyclosporin A, Magrabar GMC®, and Citronella oil.

Another embodiment comprises cyclosporin A, Magrabar GMO-CK®, and Citronella oil.

Another embodiment comprises cyclosporin A, Magrabar MDG-5050®, and Citronella oil.

Another embodiment comprises cyclosporin A, Magrabar PGO-315®, and Citronella oil.

Another embodiment comprises cyclosporin A, Magrabar PGO-1010®, and Citronella oil.

Another embodiment comprises cyclosporin A, Mazol 300K®, and Citronella oil.

Another embodiment comprises cyclosporin A, Mazol GMO-K®, and Citronella oil.

Another embodiment comprises cyclosporin A, Mazol GMS-K®, and Citronella oil.

Another embodiment comprises cyclosporin A, Mazol PG031-K®, and Citronella oil.

Another embodiment comprises cyclosporin A, Miglyol 812®, and Citronella oil.

Another embodiment comprises cyclosporin A, Norfox 165C®, and Citronella oil.

Another embodiment comprises cyclosporin A, Schercemol GMIS®, and Citronella oil.

Another embodiment comprises cyclosporin A, Tegin®, and Citronella oil.

Another embodiment comprises cyclosporin A, Tegin 4100 Pellets®, and Citronella oil.

Another embodiment comprises cyclosporin A, Tegin M Pellets®, and Citronella oil.

Another embodiment comprises cyclosporin A, Tegin OV®, and Citronella oil.

Another embodiment comprises cyclosporin A, Teginacid H®, and Citronella oil.

Another embodiment comprises cyclosporin A, Tego Cosmo P813®, and Citronella oil.

Another embodiment comprises cyclosporin A, Wickenol 535®, and Citronella oil.

Another embodiment comprises cyclosporin A, Witconol 14®, and Citronella oil.

Another embodiment comprises cyclosporin A, Witconol 14®, and Citronella oil.

Another embodiment comprises cyclosporin A, Witconol 14F®, and Citronella oil.

Another embodiment comprises cyclosporin A, Witconol 18L®, and Citronella oil.

Another embodiment comprises cyclosporin A, Witconol GOT®, and Citronella oil.

Another embodiment comprises cyclosporin A, Witconol MST®, and Citronella oil.

Another embodiment comprises cyclosporin A, Witconol RHT®, and Citronella oil.

Another embodiment comprises cyclosporin A, a Glycol Ester, and Citronella oil.

Another embodiment comprises cyclosporin A, Alkamuls 600 DO®, and Citronella oil.

Another embodiment comprises cyclosporin A, Alkamuls SEG®, and Citronella oil.

Another embodiment comprises cyclosporin A, Atlas EM-2®, and Citronella oil.

Another embodiment comprises cyclosporin A, Cerasynt IP®, and Citronella oil.

Another embodiment comprises cyclosporin A, Cerasynt M®, and Citronella oil.

Another embodiment comprises cyclosporin A, Cerasynt MN®, and Citronella oil.

Another embodiment comprises cyclosporin A, Cerasynt PA®, and Citronella oil.

Another embodiment comprises cyclosporin A, Chemsperse EGDS®, and Citronella oil.

Another embodiment comprises cyclosporin A, Chemsperse EGMS®, and Citronella oil.

Another embodiment comprises cyclosporin A, Colonial Monolaurin®, and Citronella oil.

Another embodiment comprises cyclosporin A, DeMuls SGE-95®, and Citronella oil.

Another embodiment comprises cyclosporin A, Eccoterge 200®, and Citronella oil.

Another embodiment comprises cyclosporin A, Emerest 2380®, and Citronella oil.

Another embodiment comprises cyclosporin A, Ethox 2610®, and Citronella oil.

Another embodiment comprises cyclosporin A, Ethox DO-9®, and Citronella oil.

Another embodiment comprises cyclosporin A, Ethox DO-14®, and Citronella oil.

Another embodiment comprises cyclosporin A, Ethox SO-9®, and Citronella oil.

Another embodiment comprises cyclosporin A, Fizul MD-318®, and Citronella oil.

Another embodiment comprises cyclosporin A, Genapol EGDS-VHP®, and Citronella oil.

Another embodiment comprises cyclosporin A, Genapol TS Powder®, and Citronella oil.

Another embodiment comprises cyclosporin A, Hostacerin WO®, and Citronella oil.

Another embodiment comprises cyclosporin A, Inversol 140®, and Citronella oil.

Another embodiment comprises cyclosporin A, Kemester 104®, and Citronella oil.

Another embodiment comprises cyclosporin A, Kemester 205®, and Citronella oil.

Another embodiment comprises cyclosporin A, Kemester 226®, and Citronella oil.

Another embodiment comprises cyclosporin A, Kemester 5221SE®, and Citronella oil.

Another embodiment comprises cyclosporin A, Kemester EGDS®, and Citronella oil.

Another embodiment comprises cyclosporin A, Lexemul EGDS®, and Citronella oil.

Another embodiment comprises cyclosporin A, Lexemul EGMS®, and Citronella oil.

Another embodiment comprises cyclosporin A, Lexemul P®, and Citronella oil.

Another embodiment comprises cyclosporin A, Lipo DGLS®, and Citronella oil.

Another embodiment comprises cyclosporin A, Lipo EGDS®, and Citronella oil.

Another embodiment comprises cyclosporin A, Lipo PGMS®, and Citronella oil.

Another embodiment comprises cyclosporin A, Liposorb S-4®, and Citronella oil.

Another embodiment comprises cyclosporin A, Liposorb TO-20®, and Citronella oil.

Another embodiment comprises cyclosporin A, Lumulse PGO®, and Citronella oil.

Another embodiment comprises cyclosporin A, Mackester EGDS®, and Citronella oil.

Another embodiment comprises cyclosporin A, Mackester EGMS®, and Citronella oil.

Another embodiment comprises cyclosporin A, Mackester GSTP®, and Citronella oil.

Another embodiment comprises cyclosporin A, Mackester Series®, and Citronella oil.

Another embodiment comprises cyclosporin A, Magrabar PDG-50®, and Citronella oil.

Another embodiment comprises cyclosporin A, Mapeg 6000 DS®, and Citronella oil.

Another embodiment comprises cyclosporin A, Marlowet 4702®, and Citronella oil.

Another embodiment comprises cyclosporin A, Monalube 305®, and Citronella oil.

Another embodiment comprises cyclosporin A, Monalube 310®, and Citronella oil.

Another embodiment comprises cyclosporin A, Monalube 315®, and Citronella oil.

Another embodiment comprises cyclosporin A, Monalube 320®, and Citronella oil.

Another embodiment comprises cyclosporin A, Monalube 325®, and Citronella oil.

Another embodiment comprises cyclosporin A, Monalube 330®, and Citronella oil.

Another embodiment comprises cyclosporin A, Naturechem PGHS®, and Citronella oil.

Another embodiment comprises cyclosporin A, Polycastorol PLO-840®, and Citronella oil.

Another embodiment comprises cyclosporin A, Polytex 10M®, and Citronella oil.

Another embodiment comprises cyclosporin A, Ritasynt IP®, and Citronella oil.

Another embodiment comprises cyclosporin A, Ross Chem PEG 600 DT®, and Citronella oil.

Another embodiment comprises cyclosporin A, Schercemol PGMS®, and Citronella oil.

Another embodiment comprises cyclosporin A, Sponto H-44C®, and Citronella oil.

Another embodiment comprises cyclosporin A, Tegin G®, and Citronella oil.

Another embodiment comprises cyclosporin A, Witbreak DGE-182®, and Citronella oil.

Another embodiment comprises cyclosporin A, Witbreak DGE-182®, and Citronella oil.

Another embodiment comprises cyclosporin A, Witbreak DRA-21®, and Citronella oil.

Another embodiment comprises cyclosporin A, Witbreak DRA-21®, and Citronella oil.

Another embodiment comprises cyclosporin A, Witbreak DRA-50®, and Citronella oil.

Another embodiment comprises cyclosporin A, Witbreak DRA-50®, and Citronella oil.

Another embodiment comprises cyclosporin A, Witconol F26-46®, and Citronella oil.

Another embodiment comprises cyclosporin A, Witconol H-32®, and Citronella oil.

Another embodiment comprises cyclosporin A, Witconol H-33®, and Citronella oil.

Another embodiment comprises cyclosporin A, Witconol H-35A®, and Citronella oil.

Another embodiment comprises cyclosporin A, Witconol RHP®, and Citronella oil.

Another embodiment comprises cyclosporin A, a Lanolin-based Derivative, and Citronella oil.

Another embodiment comprises cyclosporin A, Amerchol CAB®, and Citronella oil.

Another embodiment comprises cyclosporin A, Amerchol L-101®, and Citronella oil.

Another embodiment comprises cyclosporin A, Amerlate LFA-LO®, and Citronella oil.

Another embodiment comprises cyclosporin A, Amerlate P®, and Citronella oil.

Another embodiment comprises cyclosporin A, Barre Common Degras®, and Citronella oil.

Another embodiment comprises cyclosporin A, Cholesterol NF®, and Citronella oil.

Another embodiment comprises cyclosporin A, Crodalan AWS®, and Citronella oil.

Another embodiment comprises cyclosporin A, Crodalan LA®, and Citronella oil.

Another embodiment comprises cyclosporin A, Emery 1650®, and Citronella oil.

Another embodiment comprises cyclosporin A, Emery 1650®, and Citronella oil.

Another embodiment comprises cyclosporin A, Emery 1740®, and Citronella oil.

Another embodiment comprises cyclosporin A, Emery 1740®, and Citronella oil.

Another embodiment comprises cyclosporin A, Forlan 500®, and Citronella oil.

Another embodiment comprises cyclosporin A, Forlan L®, and Citronella oil.

Another embodiment comprises cyclosporin A, Laneto 50®, and Citronella oil.

Another embodiment comprises cyclosporin A, Laneto 100®, and Citronella oil.

Another embodiment comprises cyclosporin A, Laneto AWS®, and Citronella oil.

Another embodiment comprises cyclosporin A, Lanfrax 1776®, and Citronella oil.

Another embodiment comprises cyclosporin A, Lanfrax 1776®, and Citronella oil.

Another embodiment comprises cyclosporin A, Lanogel 21®, and Citronella oil.

Another embodiment comprises cyclosporin A, Lipolan®, and Citronella oil.

Another embodiment comprises cyclosporin A, Lipolan 31®, and Citronella oil.

Another embodiment comprises cyclosporin A, OHlan®, and Citronella oil.

Another embodiment comprises cyclosporin A, Polychol 5®, and Citronella oil.

Another embodiment comprises cyclosporin A, Polychol 15®, and Citronella oil.

Another embodiment comprises cyclosporin A, Ritacetyl®, and Citronella oil.

Another embodiment comprises cyclosporin A, Ritachol®, and Citronella oil.

Another embodiment comprises cyclosporin A, Ritahydrox®, and Citronella oil.

Another embodiment comprises cyclosporin A, Ritalafa®, and Citronella oil.

Another embodiment comprises cyclosporin A, Ritalan®, and Citronella oil.

Another embodiment comprises cyclosporin A, Ritalan AWS®, and Citronella oil.

Another embodiment comprises cyclosporin A, Ritalan C®, and Citronella oil.

Another embodiment comprises cyclosporin A, Ritawax®, and Citronella oil.

Another embodiment comprises cyclosporin A, Ritawax AEO®, and Citronella oil.

Another embodiment comprises cyclosporin A, Ritawax ALA®, and Citronella oil.

Another embodiment comprises cyclosporin A, Solan/Solan 50/Super Solan®, and Citronella oil.

Another embodiment comprises cyclosporin A, Super Hartolan/Hartolan®, and Citronella oil.

Another embodiment comprises cyclosporin A, Supersat AWS-4®, and Citronella oil.

Another embodiment comprises cyclosporin A, Supersat AWS-24®, and Citronella oil.

Another embodiment comprises cyclosporin A, a Lecithin or Lecithin Derivative, and Citronella oil.

Another embodiment comprises cyclosporin A, Alcolec®, and Citronella oil.

Another embodiment comprises cyclosporin A, Lecithin®, and Citronella oil.

Another embodiment comprises cyclosporin A, Lexin K®, and Citronella oil.

Another embodiment comprises cyclosporin A, Natipide®, and Citronella oil.

Another embodiment comprises cyclosporin A, a Lignin or Lignin Derivative, and Citronella oil.

Another embodiment comprises cyclosporin A, Diwatex XP 9®, and Citronella oil.

Another embodiment comprises cyclosporin A, Dynasperse LCD®, and Citronella oil.

Another embodiment comprises cyclosporin A, Indulin SAL®, and Citronella oil.

Another embodiment comprises cyclosporin A, Indulin W-1®, and Citronella oil.

Another embodiment comprises cyclosporin A, Indulin W-5®, and Citronella oil.

Another embodiment comprises cyclosporin A, Lignosol FTA®, and Citronella oil.

Another embodiment comprises cyclosporin A, Lignosol SFX-65®, and Citronella oil.

Another embodiment comprises cyclosporin A, Marasperse 52 CP®, and Citronella oil.

Another embodiment comprises cyclosporin A, Marasperse AG®, and Citronella oil.

Another embodiment comprises cyclosporin A, Marasperse CBOS-4®, and Citronella oil.

Another embodiment comprises cyclosporin A, Ufoxane 2®, and Citronella oil.

Another embodiment comprises cyclosporin A, a Methyl Ester, and Citronella oil.

Another embodiment comprises cyclosporin A, E.B. Cleaner AK®, and Citronella oil.

Another embodiment comprises cyclosporin A, Oleocal ME-70®, and Citronella oil.

Another embodiment comprises cyclosporin A, Trade Name Company®, and Citronella oil.

Another embodiment comprises cyclosporin A, Methyl Esters (cont'd)®, and Citronella oil.

Another embodiment comprises cyclosporin A, Oleocal ME-92®, and Citronella oil.

Another embodiment comprises cyclosporin A, Oleocal ME-112®, and Citronella oil.

Another embodiment comprises cyclosporin A, Oleocal ME-130®, and Citronella oil.

Another embodiment comprises cyclosporin A, a Monoglyceride or a Derivative thereof, and Citronella oil.

Another embodiment comprises cyclosporin A, Dynacet 211®, and Citronella oil.

Another embodiment comprises cyclosporin A, Hetsorb S-20®, and Citronella oil.

Another embodiment comprises cyclosporin A, Imwitor 191®, and Citronella oil.

Another embodiment comprises cyclosporin A, Imwitor 370®, and Citronella oil.

Another embodiment comprises cyclosporin A, Imwitor 375®, and Citronella oil.

Another embodiment comprises cyclosporin A, Imwitor 900®, and Citronella oil.

Another embodiment comprises cyclosporin A, Imwitor 945®, and Citronella oil.

Another embodiment comprises cyclosporin A, Imwitor 2020®, and Citronella oil.

Another embodiment comprises cyclosporin A, Kemester 5500®, and Citronella oil.

Another embodiment comprises cyclosporin A, Kemester 6000®, and Citronella oil.

Another embodiment comprises cyclosporin A, Magrabar GMC®, and Citronella oil.

Another embodiment comprises cyclosporin A, Magrabar GMO-CK®, and Citronella oil.

Another embodiment comprises cyclosporin A, Magrabar GPC-10®, and Citronella oil.

Another embodiment comprises cyclosporin A, Magrabar MDG-5050®, and Citronella oil.

Another embodiment comprises cyclosporin A, Monalube 335®, and Citronella oil.

Another embodiment comprises cyclosporin A, Monoglycerides Glyceryl Monestearate Archer Daniels Midland Company®, and Citronella oil.

Another embodiment comprises cyclosporin A, Rita GMS®, and Citronella oil.

Another embodiment comprises cyclosporin A, Ritamulse SCG®, and Citronella oil.

Another embodiment comprises cyclosporin A, Softigen 701®, and Citronella oil.

Another embodiment comprises cyclosporin A, Tally 100 Plus®, and Citronella oil.

Another embodiment comprises cyclosporin A, a Polyethylene Glycol, and Citronella oil.

Another embodiment comprises cyclosporin A, Emulgade PL 68/50®, and Citronella oil.

Another embodiment comprises cyclosporin A, Lumulse PEG®, and Citronella oil.

Another embodiment comprises cyclosporin A, Rhodasurf PEG-400®, and Citronella oil.

Another embodiment comprises cyclosporin A, Rhodasurf PEG-600®, and Citronella oil.

Another embodiment comprises cyclosporin A, Witconol PEG-400®, and Citronella oil.

Another embodiment comprises cyclosporin A, a Polymeric Surfactant, and Citronella oil.

Another embodiment comprises cyclosporin A, Acritamer PNC-EG®, and Citronella oil.

Another embodiment comprises cyclosporin A, Ag-Rho DEP-775®, and Citronella oil.

Another embodiment comprises cyclosporin A, APG 325N Glycoside®, and Citronella oil.

Another embodiment comprises cyclosporin A, Aristoflex AVC®, and Citronella oil.

Another embodiment comprises cyclosporin A, Aristoflex HMB®, and Citronella oil.

Another embodiment comprises cyclosporin A, Burco NPS-225®, and Citronella oil.

Another embodiment comprises cyclosporin A, Burco NPS-816®, and Citronella oil.

Another embodiment comprises cyclosporin A, Chemccinate 5603®, and Citronella oil.

Another embodiment comprises cyclosporin A, Cosmedia Guar C-261N®, and Citronella oil.

Another embodiment comprises cyclosporin A, Gantrez S-95®, and Citronella oil.

Another embodiment comprises cyclosporin A, Glucopon 220 UP®, and Citronella oil.

Another embodiment comprises cyclosporin A, Glucopon 225 DK®, and Citronella oil.

Another embodiment comprises cyclosporin A, Glucopon 425 N®, and Citronella oil.

Another embodiment comprises cyclosporin A, Glucopon 600 UP®, and Citronella oil.

Another embodiment comprises cyclosporin A, Glucopon 625 UP®, and Citronella oil.

Another embodiment comprises cyclosporin A, Pemulen 1621®, and Citronella oil.

Another embodiment comprises cyclosporin A, Pemulen 1622®, and Citronella oil.

Another embodiment comprises cyclosporin A, Pemulen TR-1®, and Citronella oil.

Another embodiment comprises cyclosporin A, Pemulen TR-2®, and Citronella oil.

Another embodiment comprises cyclosporin A, Plantacare 818®, and Citronella oil.

Another embodiment comprises cyclosporin A, Plantapon LGC Sorb®, and Citronella oil.

Another embodiment comprises cyclosporin A, Plantaren 1200N®, and Citronella oil.

Another embodiment comprises cyclosporin A, Plantaren 2000N®, and Citronella oil.

Another embodiment comprises cyclosporin A, Viscolam AT 64®, and Citronella oil.

Another embodiment comprises cyclosporin A, Viscolam AT 64P®, and Citronella oil.

Another embodiment comprises cyclosporin A, Viscolam AT 100®, and Citronella oil.

Another embodiment comprises cyclosporin A, Viscolam MAC 7®, and Citronella oil.

Another embodiment comprises cyclosporin A, Viscolam SMC 20®, and Citronella oil.

Another embodiment comprises cyclosporin A, Witbreak RTC-323®, and Citronella oil.

Another embodiment comprises cyclosporin A, WSI 3700®, and Citronella oil.

Another embodiment comprises cyclosporin A, a Propoxylated or Ethoxylated Fatty Acid, Alcohol, or Alkyl Phenol, and Citronella oil.

Another embodiment comprises cyclosporin A, Antarox AA-60®, and Citronella oil.

Another embodiment comprises cyclosporin A, Antarox LF-224®, and Citronella oil.

Another embodiment comprises cyclosporin A, Burcomul DFE-45®, and Citronella oil.

Another embodiment comprises cyclosporin A, Burcoterge LFE-1000®, and Citronella oil.

Another embodiment comprises cyclosporin A, Chemal LF-25B®, and Citronella oil.

Another embodiment comprises cyclosporin A, Chemal LF-40B®, and Citronella oil.

Another embodiment comprises cyclosporin A, Dehypon LS-36®, and Citronella oil.

Another embodiment comprises cyclosporin A, Dehypon LS-36®, and Citronella oil.

Another embodiment comprises cyclosporin A, Dehypon LS-54®, and Citronella oil.

Another embodiment comprises cyclosporin A, Dehypon LS-54®, and Citronella oil.

Another embodiment comprises cyclosporin A, Delonic 100 VLF®, and Citronella oil.

Another embodiment comprises cyclosporin A, Delonic LF-60 MOD®, and Citronella oil.

Another embodiment comprises cyclosporin A, Empiderm B®, and Citronella oil.

Another embodiment comprises cyclosporin A, Ethylan 1206®, and Citronella oil.

Another embodiment comprises cyclosporin A, Ethylan NS-500K®, and Citronella oil.

Another embodiment comprises cyclosporin A, Ethylan NS-500LQ®, and Citronella oil.

Another embodiment comprises cyclosporin A, Genapol 1392®, and Citronella oil.

Another embodiment comprises cyclosporin A, Genapol 2317®, and Citronella oil.

Another embodiment comprises cyclosporin A, Genapol 26EP710®, and Citronella oil.

Another embodiment comprises cyclosporin A, Genapol EP 1022®, and Citronella oil.

Another embodiment comprises cyclosporin A, Genapol EP 1024®, and Citronella oil.

Another embodiment comprises cyclosporin A, Genapol EP 6068®, and Citronella oil.

Another embodiment comprises cyclosporin A, Genapol NP915®, and Citronella oil.

Another embodiment comprises cyclosporin A, Kieralon MFB®, and Citronella oil.

Another embodiment comprises cyclosporin A, Lumisolve CSA-80 V®, and Citronella oil.

Another embodiment comprises cyclosporin A, Marlowet 5001®, and Citronella oil.

Another embodiment comprises cyclosporin A, Marlox FK 64®, and Citronella oil.

Another embodiment comprises cyclosporin A, Marlox MO 124®, and Citronella oil.

Another embodiment comprises cyclosporin A, Marlox S 58®, and Citronella oil.

Another embodiment comprises cyclosporin A, Nonatell 1003®, and Citronella oil.

Another embodiment comprises cyclosporin A, Nonatell 1038®, and Citronella oil.

Another embodiment comprises cyclosporin A, Nonatell 1052®, and Citronella oil.

Another embodiment comprises cyclosporin A, Nonatell 1061®, and Citronella oil.

Another embodiment comprises cyclosporin A, Nonatell 1075®, and Citronella oil.

Another embodiment comprises cyclosporin A, Nonatell 1088®, and Citronella oil.

Another embodiment comprises cyclosporin A, Nonatell 1123®, and Citronella oil.

Another embodiment comprises cyclosporin A, Nonatell 1153®, and Citronella oil.

Another embodiment comprises cyclosporin A, Nonatell 1161®, and Citronella oil.

Another embodiment comprises cyclosporin A, Nonatell 1172®, and Citronella oil.

Another embodiment comprises cyclosporin A, Nonatell 1181®, and Citronella oil.

Another embodiment comprises cyclosporin A, Norfox 36®, and Citronella oil.

Another embodiment comprises cyclosporin A, Procetyl AWS®, and Citronella oil.

Another embodiment comprises cyclosporin A, Sandoxylate SX 412®, and Citronella oil.

Another embodiment comprises cyclosporin A, Sandoxylate SX 418®, and Citronella oil.

Another embodiment comprises cyclosporin A, Surfonic JL-80X®, and Citronella oil.

Another embodiment comprises cyclosporin A, Surfonic JL-80X-B1®, and Citronella oil.

Another embodiment comprises cyclosporin A, Surfonic L4-29X®, and Citronella oil.

Another embodiment comprises cyclosporin A, Surfonic LF®, and Citronella oil.

Another embodiment comprises cyclosporin A, T-Det A826®, and Citronella oil.

Another embodiment comprises cyclosporin A, T-Det LF-416®, and Citronella oil.

Another embodiment comprises cyclosporin A, Tergitol Min-Foam 1X®, and Citronella oil.

Another embodiment comprises cyclosporin A, Tergitol Min-Foam 2X®, and Citronella oil.

Another embodiment comprises cyclosporin A, Triton CF-21®, and Citronella oil.

Another embodiment comprises cyclosporin A, Triton CF-76®, and Citronella oil.

Another embodiment comprises cyclosporin A, Triton XL-80N®, and Citronella oil.

Another embodiment comprises cyclosporin A, Witconol NS-98®, and Citronella oil.

Another embodiment comprises cyclosporin A, Witconol NS-108LCQ®, and Citronella oil.

Another embodiment comprises cyclosporin A, Witconol NS-145®, and Citronella oil.

Another embodiment comprises cyclosporin A, Witconol NS-179®, and Citronella oil.

Another embodiment comprises cyclosporin A, a Protein-based Surfactant, and Citronella oil.

Another embodiment comprises cyclosporin A, AminoFoam W®, and Citronella oil.

Another embodiment comprises cyclosporin A, Amiter LGOD-2®, and Citronella oil.

Another embodiment comprises cyclosporin A, Amiter LGS-2®, and Citronella oil.

Another embodiment comprises cyclosporin A, Amiter LGS-5®, and Citronella oil.

Another embodiment comprises cyclosporin A, Lamepon S®, and Citronella oil.

Another embodiment comprises cyclosporin A, Lamepon S®, and Citronella oil.

Another embodiment comprises cyclosporin A, Maypon 4C®, and Citronella oil.

Another embodiment comprises cyclosporin A, May-Tein C®, and Citronella oil.

Another embodiment comprises cyclosporin A, May-Tein CT®, and Citronella oil.

Another embodiment comprises cyclosporin A, May-Tein KTS®, and Citronella oil.

Another embodiment comprises cyclosporin A, May-Tein SY®, and Citronella oil.

Another embodiment comprises cyclosporin A, Plantapon S®, and Citronella oil.

Another embodiment comprises cyclosporin A, Proteol APL®, and Citronella oil.

Another embodiment comprises cyclosporin A, Proteol OAT®, and Citronella oil.

Another embodiment comprises cyclosporin A, Pyroter CPI-40®, and Citronella oil.

Another embodiment comprises cyclosporin A, Pyroter GPI-25®, and Citronella oil.

Another embodiment comprises cyclosporin A, Supro-Tein S®, and Citronella oil.

Another embodiment comprises cyclosporin A, Supro-Tein V®, and Citronella oil.

Another embodiment comprises cyclosporin A,® and Citronella oil.

Another embodiment comprises cyclosporin A, a Sarcosine Derivative®, and Citronella oil.

Another embodiment comprises cyclosporin A, Crodasinic LS-30®, and Citronella oil.

Another embodiment comprises cyclosporin A, Vanseal CS®, and Citronella oil.

Another embodiment comprises cyclosporin A, Vanseal LS®, and Citronella oil.

Another embodiment comprises cyclosporin A, Vanseal MS®, and Citronella oil.

Another embodiment comprises cyclosporin A, Vanseal NACS-30®, and Citronella oil.

Another embodiment comprises cyclosporin A, Vanseal NALS-95®, and Citronella oil.

Another embodiment comprises cyclosporin A, Vanseal OS®, and Citronella oil.

Another embodiment comprises cyclosporin A,® and Citronella oil.

Another embodiment comprises cyclosporin A, a Silicone-based Surfactant®, and Citronella oil.

Another embodiment comprises cyclosporin A, Abil-B-9950®, and Citronella oil.

Another embodiment comprises cyclosporin A, Abil Care 85®, and Citronella oil.

Another embodiment comprises cyclosporin A, Abil EM 90®, and Citronella oil.

Another embodiment comprises cyclosporin A, Abil EM 97®, and Citronella oil.

Another embodiment comprises cyclosporin A, Abil WE-09®, and Citronella oil.

Another embodiment comprises cyclosporin A, Dow Corning 124®Fluid®, and Citronella oil.

Another embodiment comprises cyclosporin A, Dow Corning 3225C®, and Citronella oil.

Another embodiment comprises cyclosporin A, Dow Corning 5200®, and Citronella oil.

Another embodiment comprises cyclosporin A, Dow Corning 04-3667®, and Citronella oil.

Another embodiment comprises cyclosporin A, Monasil PCA®, and Citronella oil.

Another embodiment comprises cyclosporin A, Monasil PDM®, and Citronella oil.

Another embodiment comprises cyclosporin A, Monasil PLN®, and Citronella oil.

Another embodiment comprises cyclosporin A, Polyderm PPI-SI-WS®, and Citronella oil.

Another embodiment comprises cyclosporin A, Troysol 380W®, and Citronella oil.

Another embodiment comprises cyclosporin A, Troysol S366®, and Citronella oil.

Another embodiment comprises cyclosporin A, a Sorbitan Derivative, and Citronella oil.

Another embodiment comprises cyclosporin A, Alkamuls SML®, and Citronella oil.

Another embodiment comprises cyclosporin A, Alkamuls SMO®, and Citronella oil.

Another embodiment comprises cyclosporin A, Alkamuls STO®, and Citronella oil.

Another embodiment comprises cyclosporin A, Arlacel 20®, and Citronella oil.

Another embodiment comprises cyclosporin A, Arlacel 40®, and Citronella oil.

Another embodiment comprises cyclosporin A, Arlacel 60®, and Citronella oil.

Another embodiment comprises cyclosporin A, Arlacel 80®, and Citronella oil.

Another embodiment comprises cyclosporin A, Arlacel C®, and Citronella oil.

Another embodiment comprises cyclosporin A, Armul 21®, and Citronella oil.

Another embodiment comprises cyclosporin A, Atlox 80®, and Citronella oil.

Another embodiment comprises cyclosporin A, Atlox 847®, and Citronella oil.

Another embodiment comprises cyclosporin A, Atlox 1045A®, and Citronella oil.

Another embodiment comprises cyclosporin A, Canarcel 20®, and Citronella oil.

Another embodiment comprises cyclosporin A, Canarcel 60®, and Citronella oil.

Another embodiment comprises cyclosporin A, Canarcel 80®, and Citronella oil.

Another embodiment comprises cyclosporin A, Canarcel TW 20®, and Citronella oil.

Another embodiment comprises cyclosporin A, Canarcel TW 60®, and Citronella oil.

Another embodiment comprises cyclosporin A, Canarcel TW 80®, and Citronella oil.

Another embodiment comprises cyclosporin A, Coladet BSB-P®, and Citronella oil.

Another embodiment comprises cyclosporin A, Customulse O-20®, and Citronella oil.

Another embodiment comprises cyclosporin A, Dehymuls E®, and Citronella oil.

Another embodiment comprises cyclosporin A, DeSotan SMO®, and Citronella oil.

Another embodiment comprises cyclosporin A, DeSotan SMO-20®, and Citronella oil.

Another embodiment comprises cyclosporin A, DeSotan SMT®, and Citronella oil.

Another embodiment comprises cyclosporin A, DeSotan SMT-20®, and Citronella oil.

Another embodiment comprises cyclosporin A, Durfax 60®, and Citronella oil.

Another embodiment comprises cyclosporin A, Durfax 65®, and Citronella oil.

Another embodiment comprises cyclosporin A, Durfax 80®, and Citronella oil.

Another embodiment comprises cyclosporin A, Durtan 60®, and Citronella oil.

Another embodiment comprises cyclosporin A, Durtan 65®, and Citronella oil.

Another embodiment comprises cyclosporin A, Liposorb L®, and Citronella oil.

Another embodiment comprises cyclosporin A, Liposorb L-10®, and Citronella oil.

Another embodiment comprises cyclosporin A, Liposorb L-20®, and Citronella oil.

Another embodiment comprises cyclosporin A, Liposorb O®, and Citronella oil.

Another embodiment comprises cyclosporin A, Liposorb O-20®, and Citronella oil.

Another embodiment comprises cyclosporin A, Liposorb P®, and Citronella oil.

Another embodiment comprises cyclosporin A, Liposorb P-20®, and Citronella oil.

Another embodiment comprises cyclosporin A, Liposorb S®, and Citronella oil.

Another embodiment comprises cyclosporin A, Liposorb S-20®, and Citronella oil.

Another embodiment comprises cyclosporin A, Liposorb SQO®, and Citronella oil.

Another embodiment comprises cyclosporin A, Liposorb TO®, and Citronella oil.

Another embodiment comprises cyclosporin A, Liposorb TS®, and Citronella oil.

Another embodiment comprises cyclosporin A, Liposorb TS-20®, and Citronella oil.

Another embodiment comprises cyclosporin A, Lumisorb PS®, and Citronella oil.

Another embodiment comprises cyclosporin A, Lumisorb SMO (T)®, and Citronella oil.

Another embodiment comprises cyclosporin A, Lumisorb SMS K®, and Citronella oil.

Another embodiment comprises cyclosporin A, Lumisorb SSO®, and Citronella oil.

Another embodiment comprises cyclosporin A, Lumisorb STS K®, and Citronella oil.

Another embodiment comprises cyclosporin A, Lumisorb STT®, and Citronella oil.

Another embodiment comprises cyclosporin A, Magrabar SMO®, and Citronella oil.

Another embodiment comprises cyclosporin A, Magrabar SMO-VEG®, and Citronella oil.

Another embodiment comprises cyclosporin A, Magrabar SMT®, and Citronella oil.

Another embodiment comprises cyclosporin A, Magrabar STO®, and Citronella oil.

Another embodiment comprises cyclosporin A, Miracare BC-27®, and Citronella oil.

Another embodiment comprises cyclosporin A, Ritabate 20®, and Citronella oil.

Another embodiment comprises cyclosporin A, Ritabate 40®, and Citronella oil.

Another embodiment comprises cyclosporin A, Ritabate 60®, and Citronella oil.

Another embodiment comprises cyclosporin A, Ritabate 80®, and Citronella oil.

Another embodiment comprises cyclosporin A, T-Maz®, and Citronella oil.

Another embodiment comprises cyclosporin A, Tego SML®, and Citronella oil.

Another embodiment comprises cyclosporin A, Tego SML 20®, and Citronella oil.

Another embodiment comprises cyclosporin A, Tego SMO 80 V®, and Citronella oil.

Another embodiment comprises cyclosporin A, Tego SMO V®, and Citronella oil.

Another embodiment comprises cyclosporin A, Tego SMS®, and Citronella oil.

Another embodiment comprises cyclosporin A, Tego STO V®, and Citronella oil.

Another embodiment comprises cyclosporin A, Tween 21®, and Citronella oil.

Another embodiment comprises cyclosporin A, Tween 40®, and Citronella oil.

Another embodiment comprises cyclosporin A, Tween 60®, and Citronella oil.

Another embodiment comprises cyclosporin A, Tween 60 K®, and Citronella oil.

Another embodiment comprises cyclosporin A, Tween 61®, and Citronella oil.

Another embodiment comprises cyclosporin A, Tween 65®, and Citronella oil.

Another embodiment comprises cyclosporin A, Tween 80®, and Citronella oil.

Another embodiment comprises cyclosporin A, Tween 80 K®, and Citronella oil.

Another embodiment comprises cyclosporin A, Tween 81®, and Citronella oil.

Another embodiment comprises cyclosporin A, Tween 85®, and Citronella oil.

Another embodiment comprises cyclosporin A, a Sucrose or Glucose Ester, or Derivative thereof, and Citronella oil.

Another embodiment comprises cyclosporin A, DeSulf GOS-P-60WCG®, and Citronella oil.

Another embodiment comprises cyclosporin A, Glucam E-20 Distearate®, and Citronella oil.

Another embodiment comprises cyclosporin A, Glucamate DOE-120®, and Citronella oil.

Another embodiment comprises cyclosporin A, Glucamate SSE-20®, and Citronella oil.

Another embodiment comprises cyclosporin A, Glucate DO®, and Citronella oil.

Another embodiment comprises cyclosporin A, Glucate SS®, and Citronella oil.

Another embodiment comprises cyclosporin A, Glucopon 425 UP®, and Citronella oil.

Another embodiment comprises cyclosporin A, Isolan IS®, and Citronella oil.

Another embodiment comprises cyclosporin A, Mazon 40®, and Citronella oil.

Another embodiment comprises cyclosporin A, Montanov 82®, and Citronella oil.

Another embodiment comprises cyclosporin A, Montanov 202®, and Citronella oil.

Another embodiment comprises cyclosporin A, Montanov S®, and Citronella oil.

Another embodiment comprises cyclosporin A, Rheozan®, and Citronella oil.

Another embodiment comprises cyclosporin A, Simulsol AS 48®, and Citronella oil.

Another embodiment comprises cyclosporin A, Simulsol SL 4®, and Citronella oil.

Another embodiment comprises cyclosporin A, Simulsol SL 10®, and Citronella oil.

Another embodiment comprises cyclosporin A, Simulsol SL 11W®, and Citronella oil.

Another embodiment comprises cyclosporin A, Simulsol SL 55®, and Citronella oil.

Another embodiment comprises cyclosporin A, Suga Nate 100 and 160®, and Citronella oil.

Another embodiment comprises cyclosporin A, Tego Care 450®, and Citronella oil.

Another embodiment comprises cyclosporin A, Tego Care CG 90®, and Citronella oil.

Another embodiment comprises cyclosporin A, Tego Care PS®, and Citronella oil.

Another embodiment comprises cyclosporin A, Tegosoft PSE 141 G®, and Citronella oil.

Another embodiment comprises cyclosporin A, Tegotens G 826®, and Citronella oil.

Another embodiment comprises cyclosporin A, Triton BG-10 (70%)®, and Citronella oil.

Another embodiment comprises cyclosporin A, Triton CG-110 (60%)®, and Citronella oil.

Another embodiment comprises cyclosporin A, Wickenol 545®, and Citronella oil.

Another embodiment comprises cyclosporin A, an alcohol, and Juniper oil.

Another embodiment comprises cyclosporin A, Diglycerol®, and Juniper oil.

Another embodiment comprises cyclosporin A, Hetoxide GT-80®, and Juniper oil.

Another embodiment comprises cyclosporin A, Lexemul BEO®, and Juniper oil.

Another embodiment comprises cyclosporin A, Polyglycerol-3®, and Juniper oil.

Another embodiment comprises cyclosporin A, Redicote E Series®, and Juniper oil.

Another embodiment comprises cyclosporin A, Simulsol OX 1005L®, and Juniper oil.

Another embodiment comprises cyclosporin A, Stanfax 567®, and Juniper oil.

Another embodiment comprises cyclosporin A, TA-1618®, and Juniper oil.

Another embodiment comprises cyclosporin A, Witconol H-31A®, and Juniper oil.

Another embodiment comprises cyclosporin A, an amine oxide, and Juniper oil.

Another embodiment comprises cyclosporin A, AO-405®, and Juniper oil.

Another embodiment comprises cyclosporin A, AO-455®, and Juniper oil.

Another embodiment comprises cyclosporin A, AO 728 Special®, and Juniper oil.

Another embodiment comprises cyclosporin A, Barlox 12®, and Juniper oil.

Another embodiment comprises cyclosporin A, Barlox 14®, and Juniper oil.

Another embodiment comprises cyclosporin A, Burcoxide Lo®, and Juniper oil.

Another embodiment comprises cyclosporin A, Caloxamine LO®, and Juniper oil.

Another embodiment comprises cyclosporin A, Chemoxide CAW®, and Juniper oil.

Another embodiment comprises cyclosporin A, Chemoxide LM-30®, and Juniper oil.

Another embodiment comprises cyclosporin A, Chemoxide LO®, and Juniper oil.

Another embodiment comprises cyclosporin A, Chemoxide MO®, and Juniper oil.

Another embodiment comprises cyclosporin A, Colalux CAO-35®, and Juniper oil.

Another embodiment comprises cyclosporin A, Colalux LO®, and Juniper oil.

Another embodiment comprises cyclosporin A, DeMox CAPO®, and Juniper oil.

Another embodiment comprises cyclosporin A, DeMox CSG-30®, and Juniper oil.

Another embodiment comprises cyclosporin A, DeMox LAO®, and Juniper oil.

Another embodiment comprises cyclosporin A, Emcol L®, and Juniper oil.

Another embodiment comprises cyclosporin A, Empigen OB®, and Juniper oil.

Another embodiment comprises cyclosporin A, Empigen OS/A®, and Juniper oil.

Another embodiment comprises cyclosporin A, Foamox CDO®, and Juniper oil.

Another embodiment comprises cyclosporin A, Foamox DMM®, and Juniper oil.

Another embodiment comprises cyclosporin A, Foamox DMS®, and Juniper oil.

Another embodiment comprises cyclosporin A, Genaminox KC®, and Juniper oil.

Another embodiment comprises cyclosporin A, Genaminox LA®, and Juniper oil.

Another embodiment comprises cyclosporin A, Hartofoam SAO®, and Juniper oil.

Another embodiment comprises cyclosporin A, Hartox DMCD®, and Juniper oil.

Another embodiment comprises cyclosporin A, Lipowax DAT®, and Juniper oil.

Another embodiment comprises cyclosporin A, Lipowax PB Pastilles®, and Juniper oil.

Another embodiment comprises cyclosporin A, Mackamine C8®, and Juniper oil.

Another embodiment comprises cyclosporin A, Mackamine C10®, and Juniper oil.

Another embodiment comprises cyclosporin A, Mackamine C14®, and Juniper oil.

Another embodiment comprises cyclosporin A, Mackamine CAO®, and Juniper oil.

Another embodiment comprises cyclosporin A, Mackamine CO®, and Juniper oil.

Another embodiment comprises cyclosporin A, Mackamine LO®, and Juniper oil.

Another embodiment comprises cyclosporin A, Mackamine O2®, and Juniper oil.

Another embodiment comprises cyclosporin A, Mackamine SAO®, and Juniper oil.

Another embodiment comprises cyclosporin A, Mackamine SO®, and Juniper oil.

Another embodiment comprises cyclosporin A, Mazox KCAO®, and Juniper oil.

Another embodiment comprises cyclosporin A, Monalac MO®, and Juniper oil.

Another embodiment comprises cyclosporin A, Norfox LDA®, and Juniper oil.

Another embodiment comprises cyclosporin A, Rhodamox LO®, and Juniper oil.

Another embodiment comprises cyclosporin A, Schercamox C-AA®, and Juniper oil.

Another embodiment comprises cyclosporin A, Schercamox DMA®, and Juniper oil.

Another embodiment comprises cyclosporin A, Schercamox DML®, and Juniper oil.

Another embodiment comprises cyclosporin A, Schercamox DMM®, and Juniper oil.

Another embodiment comprises cyclosporin A, Schercamox DMS®, and Juniper oil.

Another embodiment comprises cyclosporin A, Tegotens DO®, and Juniper oil.

Another embodiment comprises cyclosporin A, Tomah AO-14-2®, and Juniper oil.

Another embodiment comprises cyclosporin A, Triaminox CDO®, and Juniper oil.

Another embodiment comprises cyclosporin A, a block polymer, and Juniper oil.

Another embodiment comprises cyclosporin A, AL 2070®, and Juniper oil.

Another embodiment comprises cyclosporin A, Antarox 17-R-2®, and Juniper oil.

Another embodiment comprises cyclosporin A, Antarox 25-R-2®, and Juniper oil.

Another embodiment comprises cyclosporin A, Antarox 31-R-1®, and Juniper oil.

Another embodiment comprises cyclosporin A, Antarox P-84®, and Juniper oil.

Another embodiment comprises cyclosporin A, Antarox P-104/H®, and Juniper oil.

Another embodiment comprises cyclosporin A, Arnox BP-Series®, and Juniper oil.

Another embodiment comprises cyclosporin A, Chemonic 435®, and Juniper oil.

Another embodiment comprises cyclosporin A, Chemonic D-25®, and Juniper oil.

Another embodiment comprises cyclosporin A, Chemonic PL Series®, and Juniper oil.

Another embodiment comprises cyclosporin A, Ethox L-121®, and Juniper oil.

Another embodiment comprises cyclosporin A, Ethox L-122®, and Juniper oil.

Another embodiment comprises cyclosporin A, Genapol PF-10®, and Juniper oil.

Another embodiment comprises cyclosporin A, Genapol PF-20®, and Juniper oil.

Another embodiment comprises cyclosporin A, Genapol PF-40A®, and Juniper oil.

Another embodiment comprises cyclosporin A, Norfox 2-LF®, and Juniper oil.

Another embodiment comprises cyclosporin A, Simulsol NW 342®, and Juniper oil.

Another embodiment comprises cyclosporin A, T-Det BP-1®, and Juniper oil.

Another embodiment comprises cyclosporin A, T-Det XD®, and Juniper oil.

Another embodiment comprises cyclosporin A, T-Det XH®, and Juniper oil.

Another embodiment comprises cyclosporin A, Triton CF-32®, and Juniper oil.

Another embodiment comprises cyclosporin A, Witconol 171®, and Juniper oil.

Another embodiment comprises cyclosporin A, Witconol 324®, and Juniper oil.

Another embodiment comprises cyclosporin A, Witconol 324D®, and Juniper oil.

Another embodiment comprises cyclosporin A, Witconol PD-2000®, and Juniper oil.

Another embodiment comprises cyclosporin A, a Carboxylated Alcohol or Alkylphenol Ethoxylate, and Juniper oil.

Another embodiment comprises cyclosporin A, Emcol CN-6®, and Juniper oil.

Another embodiment comprises cyclosporin A, Ethcarb®, and Juniper oil.

Another embodiment comprises cyclosporin A, Gemtex WNT-Conc®, and Juniper oil.

Another embodiment comprises cyclosporin A, Incrodet TD7-C®, and Juniper oil.

Another embodiment comprises cyclosporin A, Marlinat CM 105/80®, and Juniper oil.

Another embodiment comprises cyclosporin A, Marlowet 1072®, and Juniper oil.

Another embodiment comprises cyclosporin A, Marlowet 4530®, and Juniper oil.

Another embodiment comprises cyclosporin A, Marlowet 4530 LF®, and Juniper oil.

Another embodiment comprises cyclosporin A, Marlowet 4534®, and Juniper oil.

Another embodiment comprises cyclosporin A, Marlowet 4538®, and Juniper oil.

Another embodiment comprises cyclosporin A, Marlowet 4539®, and Juniper oil.

Another embodiment comprises cyclosporin A, Marlowet 4539 LF®, and Juniper oil.

Another embodiment comprises cyclosporin A, Marlowet 4541®, and Juniper oil.

Another embodiment comprises cyclosporin A, Miranate LEC-80®, and Juniper oil.

Another embodiment comprises cyclosporin A, Sandopan B®, and Juniper oil.

Another embodiment comprises cyclosporin A, Sandopan B Modified®, and Juniper oil.

Another embodiment comprises cyclosporin A, Sandopan LS-24 Gel®, and Juniper oil.

Another embodiment comprises cyclosporin A, Surfine T-A®, and Juniper oil.

Another embodiment comprises cyclosporin A, a Carboxylic Acid/Fatty Acid, and Juniper oil.

Another embodiment comprises cyclosporin A, Colaterge RAM®, and Juniper oil.

Another embodiment comprises cyclosporin A, Colatrope INC®, and Juniper oil.

Another embodiment comprises cyclosporin A, Crodacid B®, and Juniper oil.

Another embodiment comprises cyclosporin A, DeTrope CA-100®, and Juniper oil.

Another embodiment comprises cyclosporin A, Latol MTO®, and Juniper oil.

Another embodiment comprises cyclosporin A, Lumulse CC-33 K®, and Juniper oil.

Another embodiment comprises cyclosporin A, Mulls 2218®, and Juniper oil.

Another embodiment comprises cyclosporin A, OL-600®, and Juniper oil.

Another embodiment comprises cyclosporin A, OL-800®, and Juniper oil.

Another embodiment comprises cyclosporin A, R-910®, and Juniper oil.

Another embodiment comprises cyclosporin A, S-210®, and Juniper oil.

Another embodiment comprises cyclosporin A, Sandopan DTC Acid®, and Juniper oil.

Another embodiment comprises cyclosporin A, Sandopan LS 24 N®, and Juniper oil.

Another embodiment comprises cyclosporin A, Sandopan MA-18®, and Juniper oil.

Another embodiment comprises cyclosporin A, an Ethoxylated Alcohol, and Juniper oil.

Another embodiment comprises cyclosporin A, Adsee 799®, and Juniper oil.

Another embodiment comprises cyclosporin A, Adsee 799®, and Juniper oil.

Another embodiment comprises cyclosporin A, Alfonic 610-3.5®, and Juniper oil.

Another embodiment comprises cyclosporin A, Alfonic 810-2®, and Juniper oil.

Another embodiment comprises cyclosporin A, Alfonic 810-6®, and Juniper oil.

Another embodiment comprises cyclosporin A, Alfonic 1012-3®, and Juniper oil.

Another embodiment comprises cyclosporin A, Alfonic 1012-5®, and Juniper oil.

Another embodiment comprises cyclosporin A, Alfonic 1216CO-1.5®, and Juniper oil.

Another embodiment comprises cyclosporin A, Alfonic 1216CO-7®, and Juniper oil.

Another embodiment comprises cyclosporin A, Alfonic 1412-3®, and Juniper oil.

Another embodiment comprises cyclosporin A, Alfonic 1412-7®, and Juniper oil.

Another embodiment comprises cyclosporin A, Arlasolve 200®, and Juniper oil.

Another embodiment comprises cyclosporin A, Arlasolve 200 Liquid®, and Juniper oil.

Another embodiment comprises cyclosporin A, Armix 180-C®, and Juniper oil.

Another embodiment comprises cyclosporin A, Armix 183®, and Juniper oil.

Another embodiment comprises cyclosporin A, Armul 2404®, and Juniper oil.

Another embodiment comprises cyclosporin A, Armul 2404®, and Juniper oil.

Another embodiment comprises cyclosporin A, Atlas EMJ-C®, and Juniper oil.

Another embodiment comprises cyclosporin A, Atlas G-2109®, and Juniper oil.

Another embodiment comprises cyclosporin A, Atlas G-3886®, and Juniper oil.

Another embodiment comprises cyclosporin A, Atlas G-3890®, and Juniper oil.

Another embodiment comprises cyclosporin A, Bio Soft E-200®, and Juniper oil.

Another embodiment comprises cyclosporin A, Bio Soft E-300®, and Juniper oil.

Another embodiment comprises cyclosporin A, Bio Soft E-400®, and Juniper oil.

Another embodiment comprises cyclosporin A, Bio Soft EN 600®, and Juniper oil.

Another embodiment comprises cyclosporin A, Bio Soft TD-400®, and Juniper oil.

Another embodiment comprises cyclosporin A, Bio Soft TD-630®, and Juniper oil.

Another embodiment comprises cyclosporin A, Brij 30®, and Juniper oil.

Another embodiment comprises cyclosporin A, Brij 52®, and Juniper oil.

Another embodiment comprises cyclosporin A, Brij 56®, and Juniper oil.

Another embodiment comprises cyclosporin A, Brij 58®, and Juniper oil.

Another embodiment comprises cyclosporin A, Brij 72®, and Juniper oil.

Another embodiment comprises cyclosporin A, Brij 76®, and Juniper oil.

Another embodiment comprises cyclosporin A, Brij 78®, and Juniper oil.

Another embodiment comprises cyclosporin A, Brij 93®, and Juniper oil.

Another embodiment comprises cyclosporin A, Brij 97®, and Juniper oil.

Another embodiment comprises cyclosporin A, Brij 98®, and Juniper oil.

Another embodiment comprises cyclosporin A, Brij 700®, and Juniper oil.

Another embodiment comprises cyclosporin A, Brij 700 S®, and Juniper oil.

Another embodiment comprises cyclosporin A, Brij 721®, and Juniper oil.

Another embodiment comprises cyclosporin A, Brij 721 S®, and Juniper oil.

Another embodiment comprises cyclosporin A, Burcoterge CDG®, and Juniper oil.

Another embodiment comprises cyclosporin A, Canasol AT 600®, and Juniper oil.

Another embodiment comprises cyclosporin A, Canasol AT 800®, and Juniper oil.

Another embodiment comprises cyclosporin A, Canasol AT 1200®, and Juniper oil.

Another embodiment comprises cyclosporin A, Canasol BJ 35®, and Juniper oil.

Another embodiment comprises cyclosporin A, Canasol BJ 36®, and Juniper oil.

Another embodiment comprises cyclosporin A, Canasol BJ 52®, and Juniper oil.

Another embodiment comprises cyclosporin A, Canasol BJ 58®, and Juniper oil.

Another embodiment comprises cyclosporin A, Canasol BJ 72®, and Juniper oil.

Another embodiment comprises cyclosporin A, Canasol BJ 78®, and Juniper oil.

Another embodiment comprises cyclosporin A, Canasol BJ 98®, and Juniper oil.

Another embodiment comprises cyclosporin A, Canasol BJ 307®, and Juniper oil.

Another embodiment comprises cyclosporin A, Cerfak 1400®, and Juniper oil.

Another embodiment comprises cyclosporin A, Cetomacrogol 1000 BP®, and Juniper oil.

Another embodiment comprises cyclosporin A, Chemonic C-2®, and Juniper oil.

Another embodiment comprises cyclosporin A, Chemonic C-10®, and Juniper oil.

Another embodiment comprises cyclosporin A, Chemonic C-20®, and Juniper oil.

Another embodiment comprises cyclosporin A, Chemonic CT-12®, and Juniper oil.

Another embodiment comprises cyclosporin A, Chemonic CT-20®, and Juniper oil.

Another embodiment comprises cyclosporin A, Chemonic CT-30®, and Juniper oil.

Another embodiment comprises cyclosporin A, Chemonic CT-55®, and Juniper oil.

Another embodiment comprises cyclosporin A, Chemonic G-7®, and Juniper oil.

Another embodiment comprises cyclosporin A, Chemonic G-26®, and Juniper oil.

Another embodiment comprises cyclosporin A, Chemonic L-4®, and Juniper oil.

Another embodiment comprises cyclosporin A, Chemonic L-7®, and Juniper oil.

Another embodiment comprises cyclosporin A, Chemonic L-12®, and Juniper oil.

Another embodiment comprises cyclosporin A, Chemonic L-23®, and Juniper oil.

Another embodiment comprises cyclosporin A, Chemonic O-2®and Juniper oil.

Another embodiment comprises cyclosporin A, Chemonic O-5®, and Juniper oil.

Another embodiment comprises cyclosporin A, Chemonic O-10®, and Juniper oil.

Another embodiment comprises cyclosporin A, Chemonic O-20®, and Juniper oil.

Another embodiment comprises cyclosporin A, Chemonic S-2®, and Juniper oil.

Another embodiment comprises cyclosporin A, Chemonic S-10®, and Juniper oil.

Another embodiment comprises cyclosporin A, Chemonic S-20®, and Juniper oil.

Another embodiment comprises cyclosporin A, Colamulse FE®, and Juniper oil.

Another embodiment comprises cyclosporin A, Cremophor A 20®, and Juniper oil.

Another embodiment comprises cyclosporin A, Cremophor SA 2®, and Juniper oil.

Another embodiment comprises cyclosporin A, Dehydol 100®, and Juniper oil.

Another embodiment comprises cyclosporin A, Dehydol O-4®, and Juniper oil.

Another embodiment comprises cyclosporin A, Delonic C-18®, and Juniper oil.

Another embodiment comprises cyclosporin A, DeSonic 6T®, and Juniper oil.

Another embodiment comprises cyclosporin A, DeSonic 9D®, and Juniper oil.

Another embodiment comprises cyclosporin A, DeSonic 9T®, and Juniper oil.

Another embodiment comprises cyclosporin A, DeSonic 12D®, and Juniper oil.

Another embodiment comprises cyclosporin A, DeSonic 12T®, and Juniper oil.

Another embodiment comprises cyclosporin A, DeSonic 15T®, and Juniper oil.

Another embodiment comprises cyclosporin A, DeSonic TDA-9®, and Juniper oil.

Another embodiment comprises cyclosporin A, DeThox GLG-7®, and Juniper oil.

Another embodiment comprises cyclosporin A, DeThox GLG-26®, and Juniper oil.

Another embodiment comprises cyclosporin A, DeThox LA-4®, and Juniper oil.

Another embodiment comprises cyclosporin A, DeThox LA-23®, and Juniper oil.

Another embodiment comprises cyclosporin A, DeThox SA-80®, and Juniper oil.

Another embodiment comprises cyclosporin A, Disponil O5®, and Juniper oil.

Another embodiment comprises cyclosporin A, Eccoterge EO-41B®, and Juniper oil.

Another embodiment comprises cyclosporin A, Empilan KA2.5/90®, and Juniper oil.

Another embodiment comprises cyclosporin A, Empilan KA5/90®, and Juniper oil.

Another embodiment comprises cyclosporin A, Empilan KM-20®, and Juniper oil.

Another embodiment comprises cyclosporin A, Empilan KM-50®, and Juniper oil.

Another embodiment comprises cyclosporin A, Empilan L-23®, and Juniper oil.

Another embodiment comprises cyclosporin A, Ethylan 25-3®, and Juniper oil.

Another embodiment comprises cyclosporin A, Ethylan 1204®, and Juniper oil.

Another embodiment comprises cyclosporin A, Ethylan DA-4®, and Juniper oil.

Another embodiment comprises cyclosporin A, Ethylan LA-230®, and Juniper oil.

Another embodiment comprises cyclosporin A, Ethylan SN®, and Juniper oil.

Another embodiment comprises cyclosporin A, Ethylan TD-60®, and Juniper oil.

Another embodiment comprises cyclosporin A, Ethylan TD-100®, and Juniper oil.

Another embodiment comprises cyclosporin A, Ethylan TD-1407®, and Juniper oil.

Another embodiment comprises cyclosporin A, Eumulgin B1®, and Juniper oil.

Another embodiment comprises cyclosporin A, Eumulgin B2®, and Juniper oil.

Another embodiment comprises cyclosporin A, Eumulgin B3®, and Juniper oil.

Another embodiment comprises cyclosporin A, Eumulgin O-10®, and Juniper oil.

Another embodiment comprises cyclosporin A, Flo Mo 80/20®, and Juniper oil.

Another embodiment comprises cyclosporin A, Flo Mo Low Foam®, and Juniper oil.

Another embodiment comprises cyclosporin A, Forlan C-24®, and Juniper oil.

Another embodiment comprises cyclosporin A, Genapol 1454®, and Juniper oil.

Another embodiment comprises cyclosporin A, Genapol BA-020®, and Juniper oil.

Another embodiment comprises cyclosporin A, Genapol BA-040®, and Juniper oil.

Another embodiment comprises cyclosporin A, Genapol C-100®, and Juniper oil.

Another embodiment comprises cyclosporin A, Genapol DA 060®, and Juniper oil.

Another embodiment comprises cyclosporin A, Genapol HS 020®, and Juniper oil.

Another embodiment comprises cyclosporin A, Genapol HS 200®, and Juniper oil.

Another embodiment comprises cyclosporin A, Genapol ID-040®, and Juniper oil.

Another embodiment comprises cyclosporin A, Genapol ID-060®, and Juniper oil.

Another embodiment comprises cyclosporin A, Genapol ID-090®, and Juniper oil.

Another embodiment comprises cyclosporin A, Genapol LA 010®, and Juniper oil.

Another embodiment comprises cyclosporin A, Genapol LA 020®, and Juniper oil.

Another embodiment comprises cyclosporin A, Genapol LA 030®, and Juniper oil.

Another embodiment comprises cyclosporin A, Genapol LA 040®, and Juniper oil.

Another embodiment comprises cyclosporin A, Genapol LA 050®, and Juniper oil.

Another embodiment comprises cyclosporin A, Genapol LA 060®, and Juniper oil.

Another embodiment comprises cyclosporin A, Genapol LA 070®, and Juniper oil.

Another embodiment comprises cyclosporin A, Genapol LA 070S®, and Juniper oil.

Another embodiment comprises cyclosporin A, Genapol LA 230®, and Juniper oil.

Another embodiment comprises cyclosporin A, Genapol O 020®, and Juniper oil.

Another embodiment comprises cyclosporin A, Genapol O 050®, and Juniper oil.

Another embodiment comprises cyclosporin A, Genapol O 100®, and Juniper oil.

Another embodiment comprises cyclosporin A, Genapol O 200®, and Juniper oil.

Another embodiment comprises cyclosporin A, Genapol SA 030®, and Juniper oil.

Another embodiment comprises cyclosporin A, Genapol SA 120®, and Juniper oil.

Another embodiment comprises cyclosporin A, Genapol T-020®, and Juniper oil.

Another embodiment comprises cyclosporin A, Genapol UD-030®, and Juniper oil.

Another embodiment comprises cyclosporin A, Genapol UD-050®, and Juniper oil.

Another embodiment comprises cyclosporin A, Genapol UD-070®, and Juniper oil.

Another embodiment comprises cyclosporin A, Genapol UD-079®, and Juniper oil.

Another embodiment comprises cyclosporin A, Genapol UD-080®, and Juniper oil.

Another embodiment comprises cyclosporin A, Genapol UD-110®, and Juniper oil.

Another embodiment comprises cyclosporin A, Genapol X 030®, and Juniper oil.

Another embodiment comprises cyclosporin A, Genapol X 050®, and Juniper oil.

Another embodiment comprises cyclosporin A, Genapol X 060®, and Juniper oil.

Another embodiment comprises cyclosporin A, Genapol X 070®, and Juniper oil.

Another embodiment comprises cyclosporin A, Genapol X 080®, and Juniper oil.

Another embodiment comprises cyclosporin A, Genapol X 100®, and Juniper oil.

Another embodiment comprises cyclosporin A, Genapol X159®, and Juniper oil.

Another embodiment comprises cyclosporin A, Generol 122 E5®, and Juniper oil.

Another embodiment comprises cyclosporin A, Generol 122 E25®, and Juniper oil.

Another embodiment comprises cyclosporin A, Hostacerin T-3®, and Juniper oil.

Another embodiment comprises cyclosporin A, Iconol LF 110®, and Juniper oil.

Another embodiment comprises cyclosporin A, Incropol CS-20®, and Juniper oil.

Another embodiment comprises cyclosporin A, Lexemul CS-20®, and Juniper oil.

Another embodiment comprises cyclosporin A, Liponic EG-1®, and Juniper oil.

Another embodiment comprises cyclosporin A, Lipowax D®, and Juniper oil.

Another embodiment comprises cyclosporin A, Lipowax G®, and Juniper oil.

Another embodiment comprises cyclosporin A, Lipowax NI®, and Juniper oil.

Another embodiment comprises cyclosporin A, Lipowax P®, and Juniper oil.

Another embodiment comprises cyclosporin A, Lipowax P-31®, and Juniper oil.

Another embodiment comprises cyclosporin A, Lipowax PR®, and Juniper oil.

Another embodiment comprises cyclosporin A, Lumulse CS-20®, and Juniper oil.

Another embodiment comprises cyclosporin A, Macol CSA-20®, and Juniper oil.

Another embodiment comprises cyclosporin A, Marlox B 24/50®, and Juniper oil.

Another embodiment comprises cyclosporin A, Mazawet 77®, and Juniper oil.

Another embodiment comprises cyclosporin A, Norfox 1713®, and Juniper oil.

Another embodiment comprises cyclosporin A, Norfox 2579®, and Juniper oil.

Another embodiment comprises cyclosporin A, Norfox Lo Foam®, and Juniper oil.

Another embodiment comprises cyclosporin A, Promulgen D®, and Juniper oil.

Another embodiment comprises cyclosporin A, Promulgen G®, and Juniper oil.

Another embodiment comprises cyclosporin A, Renex 30®, and Juniper oil.

Another embodiment comprises cyclosporin A, Renex 36®, and Juniper oil.

Another embodiment comprises cyclosporin A, Rhodasurf A 24®, and Juniper oil.

Another embodiment comprises cyclosporin A, Rhodasurf AAE-10®, and Juniper oil.

Another embodiment comprises cyclosporin A, Rhodasurf BEH-25®, and Juniper oil.

Another embodiment comprises cyclosporin A, Rhodasurf BEH-40®, and Juniper oil.

Another embodiment comprises cyclosporin A, Rhodasurf DA 530®, and Juniper oil.

Another embodiment comprises cyclosporin A, Rhodasurf DA 630®, and Juniper oil.

Another embodiment comprises cyclosporin A, Rhodasurf DA 639®, and Juniper oil.

Another embodiment comprises cyclosporin A, Rhodasurf LAN-23®, and Juniper oil.

Another embodiment comprises cyclosporin A, Rhodasurf ON-870®, and Juniper oil.

Another embodiment comprises cyclosporin A, Rhodasurf ON-877®, and Juniper oil.

Another embodiment comprises cyclosporin A, Rhodasurf TB-970 FLK®, and Juniper oil.

Another embodiment comprises cyclosporin A, Ritacet-20®, and Juniper oil.

Another embodiment comprises cyclosporin A, Ritachol 1000®, and Juniper oil.

Another embodiment comprises cyclosporin A, Ritachol 2000®, and Juniper oil.

Another embodiment comprises cyclosporin A, Ritachol 5000®, and Juniper oil.

Another embodiment comprises cyclosporin A, Ritox 35®, and Juniper oil.

Another embodiment comprises cyclosporin A, Surfonic DA-4®, and Juniper oil.

Another embodiment comprises cyclosporin A, Surfonic DA-6®, and Juniper oil.

Another embodiment comprises cyclosporin A, Surfonic L46-7®, and Juniper oil.

Another embodiment comprises cyclosporin A, Surfonic POA®, and Juniper oil.

Another embodiment comprises cyclosporin A, Synthrapol KB®, and Juniper oil.

Another embodiment comprises cyclosporin A, Teginacid®, and Juniper oil.

Another embodiment comprises cyclosporin A, Teginacid C®, and Juniper oil.

Another embodiment comprises cyclosporin A, Tegotens EC 11®, and Juniper oil.

Another embodiment comprises cyclosporin A, Tinegal NA®, and Juniper oil.

Another embodiment comprises cyclosporin A, Tomadol 400®, and Juniper oil.

Another embodiment comprises cyclosporin A, Tomadol 600®, and Juniper oil.

Another embodiment comprises cyclosporin A, Tomadol 900®, and Juniper oil.

Another embodiment comprises cyclosporin A, Uniperol O®, and Juniper oil.

Another embodiment comprises cyclosporin A, Witconol SN Series®, and Juniper oil.

Another embodiment comprises cyclosporin A, an Ethoxylated Alkylphenol, and Juniper oil.

Another embodiment comprises cyclosporin A, Antarox LF-222®, and Juniper oil.

Another embodiment comprises cyclosporin A, Atlox 775®, and Juniper oil.

Another embodiment comprises cyclosporin A, Caloxylate N-9®, and Juniper oil.

Another embodiment comprises cyclosporin A, Canasol NF-1000®, and Juniper oil.

Another embodiment comprises cyclosporin A, Canasol NF-3000®, and Juniper oil.

Another embodiment comprises cyclosporin A, Canasol NF-3070®, and Juniper oil.

Another embodiment comprises cyclosporin A, Canasol OF 1670®, and Juniper oil.

Another embodiment comprises cyclosporin A, Canasol OF 2570®, and Juniper oil.

Another embodiment comprises cyclosporin A, Canasol OF 4070®, and Juniper oil.

Another embodiment comprises cyclosporin A, Chemax DNP-8®, and Juniper oil.

Another embodiment comprises cyclosporin A, Chemax DNP-18®, and Juniper oil.

Another embodiment comprises cyclosporin A, Chemax DNP-150/50®, and Juniper oil.

Another embodiment comprises cyclosporin A, DeSonic 1.5N®, and Juniper oil.

Another embodiment comprises cyclosporin A, DeSonic 4N®, and Juniper oil.

Another embodiment comprises cyclosporin A, DeSonic 5N®, and Juniper oil.

Another embodiment comprises cyclosporin A, DeSonic 6D®, and Juniper oil.

Another embodiment comprises cyclosporin A, DeSonic 6N®, and Juniper oil.

Another embodiment comprises cyclosporin A, DeSonic 7N®, and Juniper oil.

Another embodiment comprises cyclosporin A, DeSonic 9N®, and Juniper oil.

Another embodiment comprises cyclosporin A, DeSonic 10D®, and Juniper oil.

Another embodiment comprises cyclosporin A, DeSonic 11N®, and Juniper oil.

Another embodiment comprises cyclosporin A, DeSonic 12N®, and Juniper oil.

Another embodiment comprises cyclosporin A, DeSonic 15N®, and Juniper oil.

Another embodiment comprises cyclosporin A, DeSonic 20N®, and Juniper oil.

Another embodiment comprises cyclosporin A, Eccoscour RC®, and Juniper oil.

Another embodiment comprises cyclosporin A, Eccoterge EO-100®, and Juniper oil.

Another embodiment comprises cyclosporin A, Emulsifier 632/90%®, and Juniper oil.

Another embodiment comprises cyclosporin A, Geronol AG-821®, and Juniper oil.

Another embodiment comprises cyclosporin A, Gradonic N-95®, and Juniper oil.

Another embodiment comprises cyclosporin A, Hetoxide NP-4®, and Juniper oil.

Another embodiment comprises cyclosporin A, Hetoxide NP-30®, and Juniper oil.

Another embodiment comprises cyclosporin A, Hostapal N-100®, and Juniper oil.

Another embodiment comprises cyclosporin A, Hostapal N-110®, and Juniper oil.

Another embodiment comprises cyclosporin A, Igepal CTA-639W®, and Juniper oil.

Another embodiment comprises cyclosporin A, Igepal DAP-9®, and Juniper oil.

Another embodiment comprises cyclosporin A, Igepal OD-410®, and Juniper oil.

Another embodiment comprises cyclosporin A, Igepal SS-837®, and Juniper oil.

Another embodiment comprises cyclosporin A, Lipocol NP-9 USP®, and Juniper oil.

Another embodiment comprises cyclosporin A, Macol DNP-10®, and Juniper oil.

Another embodiment comprises cyclosporin A, Marlophen NP 5®, and Juniper oil.

Another embodiment comprises cyclosporin A, Marlophen P 1®, and Juniper oil.

Another embodiment comprises cyclosporin A, Surfonic NB®, and Juniper oil.

Another embodiment comprises cyclosporin A, Surfonic OPB-307®, and Juniper oil.

Another embodiment comprises cyclosporin A, Surfonic OPB-407®, and Juniper oil.

Another embodiment comprises cyclosporin A, Syn Fac 334®, and Juniper oil.

Another embodiment comprises cyclosporin A, Syn Fac 8216®, and Juniper oil.

Another embodiment comprises cyclosporin A, Triton N-57®, and Juniper oil.

Another embodiment comprises cyclosporin A, Trycol 6956®, and Juniper oil.

Another embodiment comprises cyclosporin A, Trycol 6961®, and Juniper oil.

Another embodiment comprises cyclosporin A, Trycol 6964®, and Juniper oil.

Another embodiment comprises cyclosporin A, Trycol 6969®, and Juniper oil.

Another embodiment comprises cyclosporin A, Trycol 6974®, and Juniper oil.

Another embodiment comprises cyclosporin A, Witbreak DRB-127®, and Juniper oil.

Another embodiment comprises cyclosporin A, Witbreak DRB-127®, and Juniper oil.

Another embodiment comprises cyclosporin A, Witconol NP Series®, and Juniper oil.

Another embodiment comprises cyclosporin A, an Ethoxylated Aryl Phenol, and Juniper oil.

Another embodiment comprises cyclosporin A, Soprophor BSU®, and Juniper oil.

Another embodiment comprises cyclosporin A, Soprophor CY/8®, and Juniper oil.

Another embodiment comprises cyclosporin A, Soprophor S/25®, and Juniper oil.

Another embodiment comprises cyclosporin A, Witconol NIO®, and Juniper oil.

Another embodiment comprises cyclosporin A, Witconol NIW®, and Juniper oil.

Another embodiment comprises cyclosporin A, Witconol S-100®, and Juniper oil.

Another embodiment comprises cyclosporin A, an Ethoxylated Fatty Acid, and Juniper oil.

Another embodiment comprises cyclosporin A, Aldo PGHMS®, and Juniper oil.

Another embodiment comprises cyclosporin A, Alkamuls TO-15/HR®, and Juniper oil.

Another embodiment comprises cyclosporin A, Armotan AL-69-66®, and Juniper oil.

Another embodiment comprises cyclosporin A, Cerasynt 840®, and Juniper oil.

Another embodiment comprises cyclosporin A, Cerasynt 945®, and Juniper oil.

Another embodiment comprises cyclosporin A, Crystal Inhibitor No. 5®, and Juniper oil.

Another embodiment comprises cyclosporin A, DeThox Acid L-9®, and Juniper oil.

Another embodiment comprises cyclosporin A, DeThox Acid S-8®, and Juniper oil.

Another embodiment comprises cyclosporin A, Ethofat 242/25®, and Juniper oil.

Another embodiment comprises cyclosporin A, Hydropalat 65®, and Juniper oil.

Another embodiment comprises cyclosporin A, Lipo EGMS®, and Juniper oil.

Another embodiment comprises cyclosporin A, Lipopeg 2 DL®, and Juniper oil.

Another embodiment comprises cyclosporin A, Lipopeg 4 DL®, and Juniper oil.

Another embodiment comprises cyclosporin A, Lipopeg 4-L®, and Juniper oil.

Another embodiment comprises cyclosporin A, Lipopeg 39-S®, and Juniper oil.

Another embodiment comprises cyclosporin A, Lipopeg 4-S®, and Juniper oil.

Another embodiment comprises cyclosporin A, Lipopeg 10-S®, and Juniper oil.

Another embodiment comprises cyclosporin A, Lipopeg 100-S®, and Juniper oil.

Another embodiment comprises cyclosporin A, Lipopeg 6000 DS®, and Juniper oil.

Another embodiment comprises cyclosporin A, Lumulse 40-L®, and Juniper oil.

Another embodiment comprises cyclosporin A, Lumulse 40-S®, and Juniper oil.

Another embodiment comprises cyclosporin A, Lumulse 42-L®, and Juniper oil.

Another embodiment comprises cyclosporin A, Lumulse 42-S®, and Juniper oil.

Another embodiment comprises cyclosporin A, Lumulse 100-S®, and Juniper oil.

Another embodiment comprises cyclosporin A, Lumulse 602-S®, and Juniper oil.

Another embodiment comprises cyclosporin A, Magrabar PGE-20-0®, and Juniper oil.

Another embodiment comprises cyclosporin A, Magrabar PGE-20L®, and Juniper oil.

Another embodiment comprises cyclosporin A, Magrabar PGE-20T®, and Juniper oil.

Another embodiment comprises cyclosporin A, Magrabar PGE-22-0®, and Juniper oil.

Another embodiment comprises cyclosporin A, Magrabar PGE-22L®, and Juniper oil.

Another embodiment comprises cyclosporin A, Magrabar PGE-22T®, and Juniper oil.

Another embodiment comprises cyclosporin A, Magrabar PGE-40-0®, and Juniper oil.

Another embodiment comprises cyclosporin A, Magrabar PGE-40L®, and Juniper oil.

Another embodiment comprises cyclosporin A, Magrabar PGE-40T®, and Juniper oil.

Another embodiment comprises cyclosporin A, Magrabar PGE-42-0®, and Juniper oil.

Another embodiment comprises cyclosporin A, Magrabar PGE-42L®, and Juniper oil.

Another embodiment comprises cyclosporin A, Magrabar PGE-42T®, and Juniper oil.

Another embodiment comprises cyclosporin A, Magrabar PGE-60-0®, and Juniper oil.

Another embodiment comprises cyclosporin A, Magrabar PGE-60L®, and Juniper oil.

Another embodiment comprises cyclosporin A, Magrabar PGE-60T®, and Juniper oil.

Another embodiment comprises cyclosporin A, Magrabar PGE-62-0®, and Juniper oil.

Another embodiment comprises cyclosporin A, Magrabar PGE-62L®, and Juniper oil.

Another embodiment comprises cyclosporin A, Magrabar PGE-62T®, and Juniper oil.

Another embodiment comprises cyclosporin A, Mapeg S-40K®, and Juniper oil.

Another embodiment comprises cyclosporin A, Marlowet OTS®, and Juniper oil.

Another embodiment comprises cyclosporin A, Naturechem PGR®, and Juniper oil.

Another embodiment comprises cyclosporin A, PG No. 4®, and Juniper oil.

Another embodiment comprises cyclosporin A, Renex 20®, and Juniper oil.

Another embodiment comprises cyclosporin A, Ritox 52®, and Juniper oil.

Another embodiment comprises cyclosporin A, Ritox 53®, and Juniper oil.

Another embodiment comprises cyclosporin A, Ritox 59®, and Juniper oil.

Another embodiment comprises cyclosporin A, Surfax 8916/A®, and Juniper oil.

Another embodiment comprises cyclosporin A, Tego Acid S 40 P®, and Juniper oil.

Another embodiment comprises cyclosporin A, Tego Acid S 100 P®, and Juniper oil.

Another embodiment comprises cyclosporin A, Tween 20®, and Juniper oil.

Another embodiment comprises cyclosporin A, Volpo 131®, and Juniper oil.

Another embodiment comprises cyclosporin A, an Ethoxylated Fatty Ester or Oil, and Juniper oil.

Another embodiment comprises cyclosporin A, Acconon 6-C10®, and Juniper oil.

Another embodiment comprises cyclosporin A, Acconon CC-6®, and Juniper oil.

Another embodiment comprises cyclosporin A, Acconon CO-7®, and Juniper oil.

Another embodiment comprises cyclosporin A, Aldosperse 40/60 FG®, and Juniper oil.

Another embodiment comprises cyclosporin A, Aldosperse ML-23®, and Juniper oil.

Another embodiment comprises cyclosporin A, Aldosperse MS-20 FG®, and Juniper oil.

Another embodiment comprises cyclosporin A, Alkamuls EL-620®, and Juniper oil.

Another embodiment comprises cyclosporin A, Alkamuls EL-719®, and Juniper oil.

Another embodiment comprises cyclosporin A, Alkamuls EL-985®, and Juniper oil.

Another embodiment comprises cyclosporin A, Arlatone G®, and Juniper oil.

Another embodiment comprises cyclosporin A, Arlatone T®, and Juniper oil.

Another embodiment comprises cyclosporin A, Atlas G-1045AO, and Juniper oil.

Another embodiment comprises cyclosporin A, Atlas G-1086®, and Juniper oil.

Another embodiment comprises cyclosporin A, Atlas G-1087®, and Juniper oil.

Another embodiment comprises cyclosporin A, Atlas G-1089®, and Juniper oil.

Another embodiment comprises cyclosporin A, Atlas G-1096®, and Juniper oil.

Another embodiment comprises cyclosporin A, Atlas G-1292®, and Juniper oil.

Another embodiment comprises cyclosporin A, Atlas G-1293®, and Juniper oil.

Another embodiment comprises cyclosporin A, Atlas G-1300®, and Juniper oil.

Another embodiment comprises cyclosporin A, Atlas G-7076®, and Juniper oil.

Another embodiment comprises cyclosporin A, Capmul EMG®, and Juniper oil.

Another embodiment comprises cyclosporin A, Chemonic CO-40®, and Juniper oil.

Another embodiment comprises cyclosporin A, Chemonic LI-3®, and Juniper oil.

Another embodiment comprises cyclosporin A, Chemonic LI-7®, and Juniper oil.

Another embodiment comprises cyclosporin A, Cirrasol GM®, and Juniper oil.

Another embodiment comprises cyclosporin A, Cremophor CO 40®, and Juniper oil.

Another embodiment comprises cyclosporin A, Cremophor CO 410®, and Juniper oil.

Another embodiment comprises cyclosporin A, Cremophor EL®, and Juniper oil.

Another embodiment comprises cyclosporin A, Cremophor GC7®, and Juniper oil.

Another embodiment comprises cyclosporin A, Cremophor RH-40®, and Juniper oil.

Another embodiment comprises cyclosporin A, Crovol A-40®, and Juniper oil.

Another embodiment comprises cyclosporin A, Crovol A-70®, and Juniper oil.

Another embodiment comprises cyclosporin A, Crovol M-70®, and Juniper oil.

Another embodiment comprises cyclosporin A, Crovol PK-70®, and Juniper oil.

Another embodiment comprises cyclosporin A, Cutina E-24®, and Juniper oil.

Another embodiment comprises cyclosporin A, Dacospin 12-R®, and Juniper oil.

Another embodiment comprises cyclosporin A, Dehymuls HRE-7®, and Juniper oil.

Another embodiment comprises cyclosporin A, DeSonic 30C®, and Juniper oil.

Another embodiment comprises cyclosporin A, DeSonic 36C®, and Juniper oil.

Another embodiment comprises cyclosporin A, DeSonic 40C®, and Juniper oil.

Another embodiment comprises cyclosporin A, Durfax 60®, and Juniper oil.

Another embodiment comprises cyclosporin A, Durfax 65®, and Juniper oil.

Another embodiment comprises cyclosporin A, Durfax 80®, and Juniper oil.

Another embodiment comprises cyclosporin A, Durfax EOM®, and Juniper oil.

Another embodiment comprises cyclosporin A, Eccoterge NF-2®, and Juniper oil.

Another embodiment comprises cyclosporin A, Emulpon CO-360®, and Juniper oil.

Another embodiment comprises cyclosporin A, Emulpon CO-550®, and Juniper oil.

Another embodiment comprises cyclosporin A, Emulsogen EL®, and Juniper oil.

Another embodiment comprises cyclosporin A, Emulsogen HCO 040®, and Juniper oil.

Another embodiment comprises cyclosporin A, Emulsogen HCO 060®, and Juniper oil.

Another embodiment comprises cyclosporin A, Emulsynt 1055®, and Juniper oil.

Another embodiment comprises cyclosporin A, Ethox 3095®, and Juniper oil.

Another embodiment comprises cyclosporin A, Eumulgin RO-40®, and Juniper oil.

Another embodiment comprises cyclosporin A, Genapol G-260®, and Juniper oil.

Another embodiment comprises cyclosporin A, Glycosperse L-20®, and Juniper oil.

Another embodiment comprises cyclosporin A, Glycosperse O-5®, and Juniper oil.

Another embodiment comprises cyclosporin A, Glycosperse O-20®, and Juniper oil.

Another embodiment comprises cyclosporin A, Glycosperse O-20 FG®, and Juniper oil.

Another embodiment comprises cyclosporin A, Glycosperse S-20®, and Juniper oil.

Another embodiment comprises cyclosporin A, Glycosperse S-20 FG®, and Juniper oil.

Another embodiment comprises cyclosporin A, Glycosperse TS-20®, and Juniper oil.

Another embodiment comprises cyclosporin A, Glycosperse TS-20 FG®, and Juniper oil.

Another embodiment comprises cyclosporin A, Hetan SL®, and Juniper oil.

Another embodiment comprises cyclosporin A, Hetan SO®, and Juniper oil.

Another embodiment comprises cyclosporin A, Hetan SS®, and Juniper oil.

Another embodiment comprises cyclosporin A, Hetoxide C-2®, and Juniper oil.

Another embodiment comprises cyclosporin A, Hetoxide C-9®, and Juniper oil.

Another embodiment comprises cyclosporin A, Hetoxide C-15®, and Juniper oil.

Another embodiment comprises cyclosporin A, Hetoxide C-25®, and Juniper oil.

Another embodiment comprises cyclosporin A, Hetoxide C-40®, and Juniper oil.

Another embodiment comprises cyclosporin A, Hetoxide C-200®, and Juniper oil.

Another embodiment comprises cyclosporin A, Hetoxide C-200-50%®, and Juniper oil.

Another embodiment comprises cyclosporin A, Hetoxide GC-30®, and Juniper oil.

Another embodiment comprises cyclosporin A, Hetoxide HC-60®, and Juniper oil.

Another embodiment comprises cyclosporin A, Ice No. 2®, and Juniper oil.

Another embodiment comprises cyclosporin A, Incrocas 30/40®, and Juniper oil.

Another embodiment comprises cyclosporin A, Lexol EC®, and Juniper oil.

Another embodiment comprises cyclosporin A, Lexol EO®, and Juniper oil.

Another embodiment comprises cyclosporin A, Lipocol HCO-40®, and Juniper oil.

Another embodiment comprises cyclosporin A, Lipocol HCO-60®, and Juniper oil.

Another embodiment comprises cyclosporin A, Lipocol O-3 Special®, and Juniper oil.

Another embodiment comprises cyclosporin A, Lipopeg 2-L®, and Juniper oil.

Another embodiment comprises cyclosporin A, Lipopeg 4-DO®, and Juniper oil.

Another embodiment comprises cyclosporin A, Lipopeg 4-DS®, and Juniper oil.

Another embodiment comprises cyclosporin A, Lipovol GTB®, and Juniper oil.

Another embodiment comprises cyclosporin A, Lonzest SML-20®, and Juniper oil.

Another embodiment comprises cyclosporin A, Lonzest SMO-20®, and Juniper oil.

Another embodiment comprises cyclosporin A, Lonzest SMS-20®, and Juniper oil.

Another embodiment comprises cyclosporin A, Lonzest STO-20®, and Juniper oil.

Another embodiment comprises cyclosporin A, Lonzest STS-20®, and Juniper oil.

Another embodiment comprises cyclosporin A, Lumulse GR-40®, and Juniper oil.

Another embodiment comprises cyclosporin A, Lumulse GRH-40®, and Juniper oil.

Another embodiment comprises cyclosporin A, Lumulse POE (7) GML®, and Juniper oil.

Another embodiment comprises cyclosporin A, Lumulse POE (12) Glyc®, and Juniper oil.

Another embodiment comprises cyclosporin A, Lumulse POE (40) MS KP®, and Juniper oil.

Another embodiment comprises cyclosporin A, Marlowet 4750®, and Juniper oil.

Another embodiment comprises cyclosporin A, Marlowet LVS®, and Juniper oil.

Another embodiment comprises cyclosporin A, Marlowet R 11®, and Juniper oil.

Another embodiment comprises cyclosporin A, Marlowet R 40®, and Juniper oil.

Another embodiment comprises cyclosporin A, Mazol 80 MGK®, and Juniper oil.

Another embodiment comprises cyclosporin A, Nonionic Emulsifier T-9®, and Juniper oil.

Another embodiment comprises cyclosporin A, Oronal LCG®, and Juniper oil.

Another embodiment comprises cyclosporin A, Polyderm PPI-CO-200®, and Juniper oil.

Another embodiment comprises cyclosporin A, Polyderm PPI-CO-40®, and Juniper oil.

Another embodiment comprises cyclosporin A, Rewoderm LI 520-70®, and Juniper oil.

Another embodiment comprises cyclosporin A, Ritapeg 150 DS®, and Juniper oil.

Another embodiment comprises cyclosporin A, Softigen 767®, and Juniper oil.

Another embodiment comprises cyclosporin A, Surfactol 318®, and Juniper oil.

Another embodiment comprises cyclosporin A, Surfactol 365®, and Juniper oil.

Another embodiment comprises cyclosporin A, Syn Lube 107®, and Juniper oil.

Another embodiment comprises cyclosporin A, Syn Lube 728®, and Juniper oil.

Another embodiment comprises cyclosporin A, Syn Lube 1632H®, and Juniper oil.

Another embodiment comprises cyclosporin A, Syn Lube 6277-A®, and Juniper oil.

Another embodiment comprises cyclosporin A, T-Det C-20®, and Juniper oil.

Another embodiment comprises cyclosporin A, T-Det C-40®, and Juniper oil.

Another embodiment comprises cyclosporin A, Tally 100 Plus®, and Juniper oil.

Another embodiment comprises cyclosporin A, Uniperol EL®, and Juniper oil.

Another embodiment comprises cyclosporin A, a Fatty Ester, and Juniper oil.

Another embodiment comprises cyclosporin A, Actralube-Syn 147®, and Juniper oil.

Another embodiment comprises cyclosporin A, Atlas G-1556®, and Juniper oil.

Another embodiment comprises cyclosporin A, Atlas G-1564®, and Juniper oil.

Another embodiment comprises cyclosporin A, Atlasol Base Oil S®, and Juniper oil.

Another embodiment comprises cyclosporin A, Base ML®, and Juniper oil.

Another embodiment comprises cyclosporin A, Base MT®, and Juniper oil.

Another embodiment comprises cyclosporin A, Cerasynt 303®, and Juniper oil.

Another embodiment comprises cyclosporin A, Dermol 1012®, and Juniper oil.

Another embodiment comprises cyclosporin A, Kemester 4000®, and Juniper oil.

Another embodiment comprises cyclosporin A, Lactipol S®, and Juniper oil.

Another embodiment comprises cyclosporin A, Magrabar PGO®, and Juniper oil.

Another embodiment comprises cyclosporin A, Mayco Base BFO®, and Juniper oil.

Another embodiment comprises cyclosporin A, Methyl Linoleate®, and Juniper oil.

Another embodiment comprises cyclosporin A, Pationic 122A®, and Juniper oil.

Another embodiment comprises cyclosporin A, Pationic 138C®, and Juniper oil.

Another embodiment comprises cyclosporin A, Pationic CSL®, and Juniper oil.

Another embodiment comprises cyclosporin A, Pationic ISL®, and Juniper oil.

Another embodiment comprises cyclosporin A, Pationic SBL®, and Juniper oil.

Another embodiment comprises cyclosporin A, Pationic SSL®, and Juniper oil.

Another embodiment comprises cyclosporin A, Ritasol®, and Juniper oil.

Another embodiment comprises cyclosporin A, Tego Alkanol CS 20®, and Juniper oil.

Another embodiment comprises cyclosporin A, Tego Alkanol L23 P®, and Juniper oil.

Another embodiment comprises cyclosporin A, Tego Alkanol S2®, and Juniper oil.

Another embodiment comprises cyclosporin A, Tego Alkanol S20 P®, and Juniper oil.

Another embodiment comprises cyclosporin A, Triemulsifier 600 MS®, and Juniper oil.

Another embodiment comprises cyclosporin A, a Glycerol Esters, and Juniper oil.

Another embodiment comprises cyclosporin A, Agro #9 Wint SBO®, and Juniper oil.

Another embodiment comprises cyclosporin A, Ahcovel Base 700®, and Juniper oil.

Another embodiment comprises cyclosporin A, Aldo HMS FG®, and Juniper oil.

Another embodiment comprises cyclosporin A, Aldo MLD®, and Juniper oil.

Another embodiment comprises cyclosporin A, Aldo MLD FG®, and Juniper oil.

Another embodiment comprises cyclosporin A, Aldo MO FG®, and Juniper oil.

Another embodiment comprises cyclosporin A, Aldo MS®, and Juniper oil.

Another embodiment comprises cyclosporin A, Aldo MS FG®, and Juniper oil.

Another embodiment comprises cyclosporin A, Aldo MS LG FG®, and Juniper oil.

Another embodiment comprises cyclosporin A, Aldo MSD®, and Juniper oil.

Another embodiment comprises cyclosporin A, Aldo MSD FG®, and Juniper oil.

Another embodiment comprises cyclosporin A, Aldosperse O-20 FG®, and Juniper oil.

Another embodiment comprises cyclosporin A, Aldosperse TS-20 FG®, and Juniper oil.

Another embodiment comprises cyclosporin A, Aldosperse TS-40 FG®, and Juniper oil.

Another embodiment comprises cyclosporin A, Arlacel 165®, and Juniper oil.

Another embodiment comprises cyclosporin A, Arlacel 186®, and Juniper oil.

Another embodiment comprises cyclosporin A, Capmul GMO®, and Juniper oil.

Another embodiment comprises cyclosporin A, Capmul GMS®, and Juniper oil.

Another embodiment comprises cyclosporin A, Caprol 3GO®, and Juniper oil.

Another embodiment comprises cyclosporin A, Caprol 3GVS®, and Juniper oil.

Another embodiment comprises cyclosporin A, Caprol 6G2S®, and Juniper oil.

Another embodiment comprises cyclosporin A, Caprol 10G40®, and Juniper oil.

Another embodiment comprises cyclosporin A, Caprol 10G100®, and Juniper oil.

Another embodiment comprises cyclosporin A, Caprol ET®, and Juniper oil.

Another embodiment comprises cyclosporin A, Caprol PGE860®, and Juniper oil.

Another embodiment comprises cyclosporin A, Cerasynt 945®, and Juniper oil.

Another embodiment comprises cyclosporin A, Cerasynt GMS®, and Juniper oil.

Another embodiment comprises cyclosporin A, Cerasynt Q®, and Juniper oil.

Another embodiment comprises cyclosporin A, Cerasynt SD®, and Juniper oil.

Another embodiment comprises cyclosporin A, Cerasynt WM®, and Juniper oil.

Another embodiment comprises cyclosporin A, Chemsperse 14®, and Juniper oil.

Another embodiment comprises cyclosporin A, Cremophor GO-32®, and Juniper oil.

Another embodiment comprises cyclosporin A, Cremophor GS11®, and Juniper oil.

Another embodiment comprises cyclosporin A, Cremophor GS-32®, and Juniper oil.

Another embodiment comprises cyclosporin A, Cutina KD-16®, and Juniper oil.

Another embodiment comprises cyclosporin A, Dehymuls PGPH®, and Juniper oil.

Another embodiment comprises cyclosporin A, Dermol DGDIS®, and Juniper oil.

Another embodiment comprises cyclosporin A, Dermol DGMIS®, and Juniper oil.

Another embodiment comprises cyclosporin A, Dermol G-76®, and Juniper oil.

Another embodiment comprises cyclosporin A, Dermol G-7DI®, and Juniper oil.

Another embodiment comprises cyclosporin A, Dermol NGDI®, and Juniper oil.

Another embodiment comprises cyclosporin A, Dermolan GLH®, and Juniper oil.

Another embodiment comprises cyclosporin A, Drewmulse GMO®, and Juniper oil.

Another embodiment comprises cyclosporin A, Drewpol 3-5-M®, and Juniper oil.

Another embodiment comprises cyclosporin A, Durlac 100 W®, and Juniper oil.

Another embodiment comprises cyclosporin A, Dur-Lo®, and Juniper oil.

Another embodiment comprises cyclosporin A, Dynasan 118®, and Juniper oil.

Another embodiment comprises cyclosporin A, EC-25®, and Juniper oil.

Another embodiment comprises cyclosporin A, EM 40®, and Juniper oil.

Another embodiment comprises cyclosporin A, Emerest 2400®, and Juniper oil.

Another embodiment comprises cyclosporin A, Emerest 2452®, and Juniper oil.

Another embodiment comprises cyclosporin A, Empilan G-26®, and Juniper oil.

Another embodiment comprises cyclosporin A, Genapol TSM®, and Juniper oil.

Another embodiment comprises cyclosporin A, Hostacerin DGI®, and Juniper oil.

Another embodiment comprises cyclosporin A, Hostacerin DGL®, and Juniper oil.

Another embodiment comprises cyclosporin A, Hostacerin DGMS®, and Juniper oil.

Another embodiment comprises cyclosporin A, Hostacerin DGSB§, and Juniper oil.

Another embodiment comprises cyclosporin A, Ice No. 2®, and Juniper oil.

Another embodiment comprises cyclosporin A, Imwitor 742®, and Juniper oil.

Another embodiment comprises cyclosporin A, Imwitor 780 K®, and Juniper oil.

Another embodiment comprises cyclosporin A, Imwitor 960 Flakes®, and Juniper oil.

Another embodiment comprises cyclosporin A, Isolan GI 34®, and Juniper oil.

Another embodiment comprises cyclosporin A, Isolan GO 33®, and Juniper oil.

Another embodiment comprises cyclosporin A, Kemester 1000®, and Juniper oil.

Another embodiment comprises cyclosporin A, Kemester 2000®, and Juniper oil.

Another embodiment comprises cyclosporin A, Kemester 2000®, and Juniper oil.

Another embodiment comprises cyclosporin A, Kemester 6000SE®, and Juniper oil.

Another embodiment comprises cyclosporin A, Lamecreme DGE 18®, and Juniper oil.

Another embodiment comprises cyclosporin A, Lexemul 515®, and Juniper oil.

Another embodiment comprises cyclosporin A, Lexemul 561®, and Juniper oil.

Another embodiment comprises cyclosporin A, Lexemul AR®, and Juniper oil.

Another embodiment comprises cyclosporin A, Lexemul AS®, and Juniper oil.

Another embodiment comprises cyclosporin A, Lexemul GDL®, and Juniper oil.

Another embodiment comprises cyclosporin A, Lexemul T®, and Juniper oil.

Another embodiment comprises cyclosporin A, Lipomulse 165®, and Juniper oil.

Another embodiment comprises cyclosporin A, Lumulse GML K®, and Juniper oil.

Another embodiment comprises cyclosporin A, Lumulse GMO K®, and Juniper oil.

Another embodiment comprises cyclosporin A, Lumulse GMR K®, and Juniper oil.

Another embodiment comprises cyclosporin A, Lumulse GMT K®, and Juniper oil.

Another embodiment comprises cyclosporin A, Magrabar GMC®, and Juniper oil.

Another embodiment comprises cyclosporin A, Magrabar GMO-CK®, and Juniper oil.

Another embodiment comprises cyclosporin A, Magrabar MDG-5050®, and Juniper oil.

Another embodiment comprises cyclosporin A, Magrabar PGO-315®, and Juniper oil.

Another embodiment comprises cyclosporin A, Magrabar PGO-1010®, and Juniper oil.

Another embodiment comprises cyclosporin A, Mazol 300K®, and Juniper oil.

Another embodiment comprises cyclosporin A, Mazol GMO-K®, and Juniper oil.

Another embodiment comprises cyclosporin A, Mazol GMS-K®, and Juniper oil.

Another embodiment comprises cyclosporin A, Mazol PG031-K®, and Juniper oil.

Another embodiment comprises cyclosporin A, Miglyol 812®, and Juniper oil.

Another embodiment comprises cyclosporin A, Norfox 165C®, and Juniper oil.

Another embodiment comprises cyclosporin A, Schercemol GMIS®, and Juniper oil.

Another embodiment comprises cyclosporin A, Tegin®, and Juniper oil.

Another embodiment comprises cyclosporin A, Tegin 4100 Pellets®, and Juniper oil.

Another embodiment comprises cyclosporin A, Tegin M Pellets®, and Juniper oil.

Another embodiment comprises cyclosporin A, Tegin OV®, and Juniper oil.

Another embodiment comprises cyclosporin A, Teginacid H®, and Juniper oil.

Another embodiment comprises cyclosporin A, Tego Cosmo P813®, and Juniper oil.

Another embodiment comprises cyclosporin A, Wickenol 535®, and Juniper oil.

Another embodiment comprises cyclosporin A, Witconol 14®, and Juniper oil.

Another embodiment comprises cyclosporin A, Witconol 14®, and Juniper oil.

Another embodiment comprises cyclosporin A, Witconol 14F®, and Juniper oil.

Another embodiment comprises cyclosporin A, Witconol 18L®, and Juniper oil.

Another embodiment comprises cyclosporin A, Witconol GOT®, and Juniper oil.

Another embodiment comprises cyclosporin A, Witconol MST®, and Juniper oil.

Another embodiment comprises cyclosporin A, Witconol RHT®, and Juniper oil.

Another embodiment comprises cyclosporin A, a Glycol Ester, and Juniper oil.

Another embodiment comprises cyclosporin A, Alkamuls 600 D®, and Juniper oil.

Another embodiment comprises cyclosporin A, Alkamuls SEG®, and Juniper oil.

Another embodiment comprises cyclosporin A, Atlas EM-2®, and Juniper oil.

Another embodiment comprises cyclosporin A, Cerasynt IP®, and Juniper oil.

Another embodiment comprises cyclosporin A, Cerasynt M®, and Juniper oil.

Another embodiment comprises cyclosporin A, Cerasynt MN®, and Juniper oil.

Another embodiment comprises cyclosporin A, Cerasynt PA®, and Juniper oil.

Another embodiment comprises cyclosporin A, Chemsperse EGDS®, and Juniper oil.

Another embodiment comprises cyclosporin A, Chemsperse EGMS®, and Juniper oil.

Another embodiment comprises cyclosporin A, Colonial Monolaurin®, and Juniper oil.

Another embodiment comprises cyclosporin A, DeMuls SGE-95®, and Juniper oil.

Another embodiment comprises cyclosporin A, Eccoterge 200®, and Juniper oil.

Another embodiment comprises cyclosporin A, Emerest 2380®, and Juniper oil.

Another embodiment comprises cyclosporin A, Ethox 2610®, and Juniper oil.

Another embodiment comprises cyclosporin A, Ethox DO-9®, and Juniper oil.

Another embodiment comprises cyclosporin A, Ethox DO-14®, and Juniper oil.

Another embodiment comprises cyclosporin A, Ethox SO-9®, and Juniper oil.

Another embodiment comprises cyclosporin A, Fizul MD-318®, and Juniper oil.

Another embodiment comprises cyclosporin A, Genapol EGDS-VHP®, and Juniper oil.

Another embodiment comprises cyclosporin A, Genapol TS Powder®, and Juniper oil.

Another embodiment comprises cyclosporin A, Hostacerin WO®, and Juniper oil.

Another embodiment comprises cyclosporin A, Inversol 140®, and Juniper oil.

Another embodiment comprises cyclosporin A, Kemester 104®, and Juniper oil.

Another embodiment comprises cyclosporin A, Kemester 205®, and Juniper oil.

Another embodiment comprises cyclosporin A, Kemester 226®, and Juniper oil.

Another embodiment comprises cyclosporin A, Kemester 5221 SE®, and Juniper oil.

Another embodiment comprises cyclosporin A, Kemester EGDS®, and Juniper oil.

Another embodiment comprises cyclosporin A, Lexemul EGDS®, and Juniper oil.

Another embodiment comprises cyclosporin A, Lexemul EGMS®, and Juniper oil.

Another embodiment comprises cyclosporin A, Lexemul P®, and Juniper oil.

Another embodiment comprises cyclosporin A, Lipo DGLS®, and Juniper oil.

Another embodiment comprises cyclosporin A, Lipo EGDS®, and Juniper oil.

Another embodiment comprises cyclosporin A, Lipo PGMS®, and Juniper oil.

Another embodiment comprises cyclosporin A, Liposorb S-4®, and Juniper oil.

Another embodiment comprises cyclosporin A, Liposorb TO-20®, and Juniper oil.

Another embodiment comprises cyclosporin A, Lumulse PGC®, and Juniper oil.

Another embodiment comprises cyclosporin A, Mackester EGDS®, and Juniper oil.

Another embodiment comprises cyclosporin A, Mackester EGMS®, and Juniper oil.

Another embodiment comprises cyclosporin A, Mackester GSTP®, and Juniper oil.

Another embodiment comprises cyclosporin A, Mackester Series®, and Juniper oil.

Another embodiment comprises cyclosporin A, Magrabar PDG-50®, and Juniper oil.

Another embodiment comprises cyclosporin A, Mapeg 6000 DS®, and Juniper oil.

Another embodiment comprises cyclosporin A, Marlowet 4702®, and Juniper oil.

Another embodiment comprises cyclosporin A, Monalube 305®, and Juniper oil.

Another embodiment comprises cyclosporin A, Monalube 310®, and Juniper oil.

Another embodiment comprises cyclosporin A, Monalube 315®, and Juniper oil.

Another embodiment comprises cyclosporin A, Monalube 320®, and Juniper oil.

Another embodiment comprises cyclosporin A, Monalube 325®, and Juniper oil.

Another embodiment comprises cyclosporin A, Monalube 330®, and Juniper oil.

Another embodiment comprises cyclosporin A, Naturechem PGHS®, and Juniper oil.

Another embodiment comprises cyclosporin A, Polycastorol PLO-840®, and Juniper oil.

Another embodiment comprises cyclosporin A, Polytex 10M®, and Juniper oil.

Another embodiment comprises cyclosporin A, Ritasynt IP®, and Juniper oil.

Another embodiment comprises cyclosporin A, Ross Chem PEG 600 DT®, and Juniper oil.

Another embodiment comprises cyclosporin A, Schercemol PGMS®, and Juniper oil.

Another embodiment comprises cyclosporin A, Sponto H-44C®, and Juniper oil.

Another embodiment comprises cyclosporin A, Tegin G®, and Juniper oil.

Another embodiment comprises cyclosporin A, Witbreak DGE-182®, and Juniper oil.

Another embodiment comprises cyclosporin A, Witbreak DGE-182®, and Juniper oil.

Another embodiment comprises cyclosporin A, Witbreak DRA-21®, and Juniper oil.

Another embodiment comprises cyclosporin A, Witbreak DRA-21®, and Juniper oil.

Another embodiment comprises cyclosporin A, Witbreak DRA-50®, and Juniper oil.

Another embodiment comprises cyclosporin A, Witbreak DRA-50®, and Juniper oil.

Another embodiment comprises cyclosporin A, Witconol F26-46®, and Juniper oil.

Another embodiment comprises cyclosporin A, Witconol H-32®, and Juniper oil.

Another embodiment comprises cyclosporin A, Witconol H-33®, and Juniper oil.

Another embodiment comprises cyclosporin A, Witconol H-35A®, and Juniper oil.

Another embodiment comprises cyclosporin A, Witconol RHP®, and Juniper oil.

Another embodiment comprises cyclosporin A, a Lanolin-based Derivative, and Juniper oil.

Another embodiment comprises cyclosporin A, Amerchol CAB®, and Juniper oil.

Another embodiment comprises cyclosporin A, Amerchol L-101®, and Juniper oil.

Another embodiment comprises cyclosporin A, Amerlate LFA-LO®, and Juniper oil.

Another embodiment comprises cyclosporin A, Amerlate P®, and Juniper oil.

Another embodiment comprises cyclosporin A, Barre Common Degras®, and Juniper oil.

Another embodiment comprises cyclosporin A, Cholesterol NF®, and Juniper oil.

Another embodiment comprises cyclosporin A, Crodalan AWS®, and Juniper oil.

Another embodiment comprises cyclosporin A, Crodalan LAP, and Juniper oil.

Another embodiment comprises cyclosporin A, Emery 1650®, and Juniper oil.

Another embodiment comprises cyclosporin A, Emery 1650®, and Juniper oil.

Another embodiment comprises cyclosporin A, Emery 1740®, and Juniper oil.

Another embodiment comprises cyclosporin A, Emery 1740®, and Juniper oil.

Another embodiment comprises cyclosporin A, Forlan 500®, and Juniper oil.

Another embodiment comprises cyclosporin A, Forlan L®, and Juniper oil.

Another embodiment comprises cyclosporin A, Laneto 50®, and Juniper oil.

Another embodiment comprises cyclosporin A, Laneto 100®, and Juniper oil.

Another embodiment comprises cyclosporin A, Laneto AWS®, and Juniper oil.

Another embodiment comprises cyclosporin A, Lanfrax 1776®, and Juniper oil.

Another embodiment comprises cyclosporin A, Lanfrax 1776®, and Juniper oil.

Another embodiment comprises cyclosporin A, Lanogel 21®, and Juniper oil.

Another embodiment comprises cyclosporin A, Lipolan®, and Juniper oil.

Another embodiment comprises cyclosporin A, Lipolan 31®, and Juniper oil.

Another embodiment comprises cyclosporin A, OHlan®, and Juniper oil.

Another embodiment comprises cyclosporin A, Polychol 5®, and Juniper oil.

Another embodiment comprises cyclosporin A, Polychol 15®, and Juniper oil.

Another embodiment comprises cyclosporin A, Ritacetyl®, and Juniper oil.

Another embodiment comprises cyclosporin A, Ritachol®, and Juniper oil.

Another embodiment comprises cyclosporin A, Ritahydrox®, and Juniper oil.

Another embodiment comprises cyclosporin A, Ritalafa®, and Juniper oil.

Another embodiment comprises cyclosporin A, Ritalan®, and Juniper oil.

Another embodiment comprises cyclosporin A, Ritalan AWS®, and Juniper oil.

Another embodiment comprises cyclosporin A, Ritalan C®, and Juniper oil.

Another embodiment comprises cyclosporin A, Ritawax®, and Juniper oil.

Another embodiment comprises cyclosporin A, Ritawax AEO®, and Juniper oil.

Another embodiment comprises cyclosporin A, Ritawax ALA®, and Juniper oil.

Another embodiment comprises cyclosporin A, Solan/Solan 50/Super Solan®, and Juniper oil.

Another embodiment comprises cyclosporin A, Super Hartolan/Hartolan®, and Juniper oil.

Another embodiment comprises cyclosporin A, Supersat AWS-4®, and Juniper oil.

Another embodiment comprises cyclosporin A, Supersat AWS-24®, and Juniper oil.

Another embodiment comprises cyclosporin A, a Lecithin or Lecithin Derivative, and Juniper oil.

Another embodiment comprises cyclosporin A, Alcolec®, and Juniper oil.

Another embodiment comprises cyclosporin A, Lecithin®, and Juniper oil.

Another embodiment comprises cyclosporin A, Lexin K®, and Juniper oil.

Another embodiment comprises cyclosporin A, Natipide®, and Juniper oil.

Another embodiment comprises cyclosporin A, a Lignin or Lignin Derivative, and Juniper oil.

Another embodiment comprises cyclosporin A, Diwatex XP 9®, and Juniper oil.

Another embodiment comprises cyclosporin A, Dynasperse LCD®, and Juniper oil.

Another embodiment comprises cyclosporin A, Indulin SAL®, and Juniper oil.

Another embodiment comprises cyclosporin A, Indulin W-1®, and Juniper oil.

Another embodiment comprises cyclosporin A, Indulin W-5®, and Juniper oil.

Another embodiment comprises cyclosporin A, Lignosol FTA®, and Juniper oil.

Another embodiment comprises cyclosporin A, Lignosol SFX-65®, and Juniper oil.

Another embodiment comprises cyclosporin A, Marasperse 52 CP®, and Juniper oil.

Another embodiment comprises cyclosporin A, Marasperse AG®, and Juniper oil.

Another embodiment comprises cyclosporin A, Marasperse CBOS-4®, and Juniper oil.

Another embodiment comprises cyclosporin A, Ufoxane 2®, and Juniper oil.

Another embodiment comprises cyclosporin A, a Methyl Ester, and Juniper oil.

Another embodiment comprises cyclosporin A, E.B. Cleaner AK®, and Juniper oil.

Another embodiment comprises cyclosporin A, Oleocal ME-70®, and Juniper oil.

Another embodiment comprises cyclosporin A, Trade Name Company®, and Juniper oil.

Another embodiment comprises cyclosporin A, Methyl Esters (cont'd)®, and Juniper oil.

Another embodiment comprises cyclosporin A, Oleocal ME-92®, and Juniper oil.

Another embodiment comprises cyclosporin A, Oleocal ME-112®, and Juniper oil.

Another embodiment comprises cyclosporin A, Oleocal ME-130®, and Juniper oil.

Another embodiment comprises cyclosporin A, a Monoglyceride or a Derivative thereof, and Juniper oil.

Another embodiment comprises cyclosporin A, Dynacet 211®, and Juniper oil.

Another embodiment comprises cyclosporin A, Hetsorb S-20®, and Juniper oil.

Another embodiment comprises cyclosporin A, Imwitor 191®, and Juniper oil.

Another embodiment comprises cyclosporin A, Imwitor 370®, and Juniper oil.

Another embodiment comprises cyclosporin A, Imwitor 375®, and Juniper oil.

Another embodiment comprises cyclosporin A, Imwitor 900®, and Juniper oil.

Another embodiment comprises cyclosporin A, Imwitor 945®, and Juniper oil.

Another embodiment comprises cyclosporin A, Imwitor 2020®, and Juniper oil.

Another embodiment comprises cyclosporin A, Kemester 5500®, and Juniper oil.

Another embodiment comprises cyclosporin A, Kemester 6000®, and Juniper oil.

Another embodiment comprises cyclosporin A, Magrabar GMC®, and Juniper oil.

Another embodiment comprises cyclosporin A, Magrabar GMO-CK®, and Juniper oil.

Another embodiment comprises cyclosporin A, Magrabar GPC-10®, and Juniper oil.

Another embodiment comprises cyclosporin A, Magrabar MDG-5050®, and Juniper oil.

Another embodiment comprises cyclosporin A, Monalube 335®, and Juniper oil.

Another embodiment comprises cyclosporin A, Monoglycerides Glyceryl Monestearate Archer Daniels Midland Company®, and Juniper oil.

Another embodiment comprises cyclosporin A, Rita GMS®, and Juniper oil.

Another embodiment comprises cyclosporin A, Ritamulse SCG®, and Juniper oil.

Another embodiment comprises cyclosporin A, Softigen 701®, and Juniper oil.

Another embodiment comprises cyclosporin A, Tally 100 Plus®, and Juniper oil.

Another embodiment comprises cyclosporin A, a Polyethylene Glycol, and Juniper oil.

Another embodiment comprises cyclosporin A, Emulgade PL 68/50®, and Juniper oil.

Another embodiment comprises cyclosporin A, Lumulse PEG®, and Juniper oil.

Another embodiment comprises cyclosporin A, Rhodasurf PEG-400®, and Juniper oil.

Another embodiment comprises cyclosporin A, Rhodasurf PEG-600®, and Juniper oil.

Another embodiment comprises cyclosporin A, Witconol PEG-400®, and Juniper oil.

Another embodiment comprises cyclosporin A, a Polymeric Surfactant, and Juniper oil.

Another embodiment comprises cyclosporin A, Acritamer PNC-EG®, and Juniper oil.

Another embodiment comprises cyclosporin A, Ag-Rho DEP-775®, and Juniper oil.

Another embodiment comprises cyclosporin A, APG 325N Glycoside®, and Juniper oil.

Another embodiment comprises cyclosporin A, Aristoflex AVC®, and Juniper oil.

Another embodiment comprises cyclosporin A, Aristoflex HMB®, and Juniper oil.

Another embodiment comprises cyclosporin A, Burco NPS-225®, and Juniper oil.

Another embodiment comprises cyclosporin A, Burco NPS-816®, and Juniper oil.

Another embodiment comprises cyclosporin A, Chemccinate 5603®, and Juniper oil.

Another embodiment comprises cyclosporin A, Cosmedia Guar C-261 N®, and Juniper oil.

Another embodiment comprises cyclosporin A, Gantrez S-95®, and Juniper oil.

Another embodiment comprises cyclosporin A, Glucopon 220 UP®, and Juniper oil.

Another embodiment comprises cyclosporin A, Glucopon 225 DK®, and Juniper oil.

Another embodiment comprises cyclosporin A, Glucopon 425 N®, and Juniper oil.

Another embodiment comprises cyclosporin A, Glucopon 600 UP®, and Juniper oil.

Another embodiment comprises cyclosporin A, Glucopon 625 UP®, and Juniper oil.

Another embodiment comprises cyclosporin A, Pemulen 1621®, and Juniper oil.

Another embodiment comprises cyclosporin A, Pemulen 1622®, and Juniper oil.

Another embodiment comprises cyclosporin A, Pemulen TR-1®, and Juniper oil.

Another embodiment comprises cyclosporin A, Pemulen TR-2®, and Juniper oil.

Another embodiment comprises cyclosporin A, Plantacare 818®, and Juniper oil.

Another embodiment comprises cyclosporin A, Plantapon LGC Sorb®, and Juniper oil.

Another embodiment comprises cyclosporin A, Plantaren 1200N®, and Juniper oil.

Another embodiment comprises cyclosporin A, Plantaren 2000N®, and Juniper oil.

Another embodiment comprises cyclosporin A, Viscolam AT 64®, and Juniper oil.

Another embodiment comprises cyclosporin A, Viscolam AT 64P®, and Juniper oil.

Another embodiment comprises cyclosporin A, Viscolam AT 100®, and Juniper oil.

Another embodiment comprises cyclosporin A, Viscolam MAC 7®, and Juniper oil.

Another embodiment comprises cyclosporin A, Viscolam SMC 20®, and Juniper oil.

Another embodiment comprises cyclosporin A, Witbreak RTC-323®, and Juniper oil.

Another embodiment comprises cyclosporin A, WSI 3700®, and Juniper oil.

Another embodiment comprises cyclosporin A, a Propoxylated or Ethoxylated Fatty Acid, Alcohol, or Alkyl Phenol, and Juniper oil.

Another embodiment comprises cyclosporin A, Antarox AA-60®, and Juniper oil.

Another embodiment comprises cyclosporin A, Antarox LF-224®, and Juniper oil.

Another embodiment comprises cyclosporin A, Burcomul DFE-45®, and Juniper oil.

Another embodiment comprises cyclosporin A, Burcoterge LFE-1000®, and Juniper oil.

Another embodiment comprises cyclosporin A, Chemal LF-25B®, and Juniper oil.

Another embodiment comprises cyclosporin A, Chemal LF-40B®, and Juniper oil.

Another embodiment comprises cyclosporin A, Dehypon LS-36®, and Juniper oil.

Another embodiment comprises cyclosporin A, Dehypon LS-36®, and Juniper oil.

Another embodiment comprises cyclosporin A, Dehypon LS-54®, and Juniper oil.

Another embodiment comprises cyclosporin A, Dehypon LS-54®, and Juniper oil.

Another embodiment comprises cyclosporin A, Delonic 100 VLF®, and Juniper oil.

Another embodiment comprises cyclosporin A, Delonic LF-60 MOD®, and Juniper oil.

Another embodiment comprises cyclosporin A, Empiderm B®, and Juniper oil.

Another embodiment comprises cyclosporin A, Ethylan 1206®, and Juniper oil.

Another embodiment comprises cyclosporin A, Ethylan NS-500K®, and Juniper oil.

Another embodiment comprises cyclosporin A, Ethylan NS-500LQ®, and Juniper oil.

Another embodiment comprises cyclosporin A, Genapol 1392®, and Juniper oil.

Another embodiment comprises cyclosporin A, Genapol 2317®, and Juniper oil.

Another embodiment comprises cyclosporin A, Genapol 26EP710®, and Juniper oil.

Another embodiment comprises cyclosporin A, Genapol EP 1022®, and Juniper oil.

Another embodiment comprises cyclosporin A, Genapol EP 1024®, and Juniper oil.

Another embodiment comprises cyclosporin A, Genapol EP 6068®, and Juniper oil.

Another embodiment comprises cyclosporin A, Genapol NP915®, and Juniper oil.

Another embodiment comprises cyclosporin A, Kieralon MFB®, and Juniper oil.

Another embodiment comprises cyclosporin A, Lumisolve CSA-80 V®, and Juniper oil.

Another embodiment comprises cyclosporin A, Marlowet 5001®, and Juniper oil.

Another embodiment comprises cyclosporin A, Marlox FK 64®, and Juniper oil.

Another embodiment comprises cyclosporin A, Marlox MO 124®, and Juniper oil.

Another embodiment comprises cyclosporin A, Marlox S 58®, and Juniper oil.

Another embodiment comprises cyclosporin A, Nonatell 1003®, and Juniper oil.

Another embodiment comprises cyclosporin A, Nonatell 1038®, and Juniper oil.

Another embodiment comprises cyclosporin A, Nonatell 1052®, and Juniper oil.

Another embodiment comprises cyclosporin A, Nonatell 1061®, and Juniper oil.

Another embodiment comprises cyclosporin A, Nonatell 1075®, and Juniper oil.

Another embodiment comprises cyclosporin A, Nonatell 1088®, and Juniper oil.

Another embodiment comprises cyclosporin A, Nonatell 1123®, and Juniper oil.

Another embodiment comprises cyclosporin A, Nonatell 1153®, and Juniper oil.

Another embodiment comprises cyclosporin A, Nonatell 1161®, and Juniper oil.

Another embodiment comprises cyclosporin A, Nonatell 1172®, and Juniper oil.

Another embodiment comprises cyclosporin A, Nonatell 1181®, and Juniper oil.

Another embodiment comprises cyclosporin A, Norfox 36®, and Juniper oil.

Another embodiment comprises cyclosporin A, Procetyl AWS®, and Juniper oil.

Another embodiment comprises cyclosporin A, Sandoxylate SX 412®, and Juniper oil.

Another embodiment comprises cyclosporin A, Sandoxylate SX 418®, and Juniper oil.

Another embodiment comprises cyclosporin A, Surfonic JL-80X®, and Juniper oil.

Another embodiment comprises cyclosporin A, Surfonic JL-80X-B1®, and Juniper oil.

Another embodiment comprises cyclosporin A, Surfonic L4-29X®, and Juniper oil.

Another embodiment comprises cyclosporin A, Surfonic LF®, and Juniper oil.

Another embodiment comprises cyclosporin A, T-Det A826®, and Juniper oil.

Another embodiment comprises cyclosporin A, T-Det LF-416®, and Juniper oil.

Another embodiment comprises cyclosporin A, Tergitol Min-Foam 1X®, and Juniper oil.

Another embodiment comprises cyclosporin A, Tergitol Min-Foam 2X®, and Juniper oil.

Another embodiment comprises cyclosporin A, Triton CF-21®, and Juniper oil.

Another embodiment comprises cyclosporin A, Triton CF-76®, and Juniper oil.

Another embodiment comprises cyclosporin A, Triton XL-80N®, and Juniper oil.

Another embodiment comprises cyclosporin A, Witconol NS-98®, and Juniper oil.

Another embodiment comprises cyclosporin A, Witconol NS-108LQ®, and Juniper oil.

Another embodiment comprises cyclosporin A, Witconol NS-145®, and Juniper oil.

Another embodiment comprises cyclosporin A, Witconol NS-179®, and Juniper oil.

Another embodiment comprises cyclosporin A, a Protein-based Surfactant, and Juniper oil.

Another embodiment comprises cyclosporin A, AminoFoam W®, and Juniper oil.

Another embodiment comprises cyclosporin A, Amiter LGOD-2®, and Juniper oil.

Another embodiment comprises cyclosporin A, Amiter LGS-2®, and Juniper oil.

Another embodiment comprises cyclosporin A, Amiter LGS-5®, and Juniper oil.

Another embodiment comprises cyclosporin A, Lamepon S®, and Juniper oil.

Another embodiment comprises cyclosporin A, Lamepon S®, and Juniper oil.

Another embodiment comprises cyclosporin A, Maypon 4C®, and Juniper oil.

Another embodiment comprises cyclosporin A, May-Tein C®, and Juniper oil.

Another embodiment comprises cyclosporin A, May-Tein CT®, and Juniper oil.

Another embodiment comprises cyclosporin A, May-Tein KTS®, and Juniper oil.

Another embodiment comprises cyclosporin A, May-Tein SY®, and Juniper oil.

Another embodiment comprises cyclosporin A, Plantapon S®, and Juniper oil.

Another embodiment comprises cyclosporin A, Proteol APL®, and Juniper oil.

Another embodiment comprises cyclosporin A, Proteol OAT®, and Juniper oil.

Another embodiment comprises cyclosporin A, Pyroter CPI-40®, and Juniper oil.

Another embodiment comprises cyclosporin A, Pyroter GPI-25®, and Juniper oil.

Another embodiment comprises cyclosporin A, Supro-Tein S®, and Juniper oil.

Another embodiment comprises cyclosporin A, Supro-Tein V®, and Juniper oil.

Another embodiment comprises cyclosporin A,® and Juniper oil.

Another embodiment comprises cyclosporin A, a Sarcosine Derivative®, and Juniper oil.

Another embodiment comprises cyclosporin A, Crodasinic LS-30®, and Juniper oil.

Another embodiment comprises cyclosporin A, Vanseal CS®, and Juniper oil.

Another embodiment comprises cyclosporin A, Vanseal LS®, and Juniper oil.

Another embodiment comprises cyclosporin A, Vanseal MS®, and Juniper oil.

Another embodiment comprises cyclosporin A, Vanseal NACS-30®, and Juniper oil.

Another embodiment comprises cyclosporin A, Vanseal NALS-95®, and Juniper oil.

Another embodiment comprises cyclosporin A, Vanseal OS®, and Juniper oil.

Another embodiment comprises cyclosporin A,® and Juniper oil.

Another embodiment comprises cyclosporin A, a Silicone-based Surfactant®, and Juniper oil.

Another embodiment comprises cyclosporin A, Abil-B-9950®, and Juniper oil.

Another embodiment comprises cyclosporin A, Abil Care 85®, and Juniper oil.

Another embodiment comprises cyclosporin A, Abil EM 90®, and Juniper oil.

Another embodiment comprises cyclosporin A, Abil EM 97®, and Juniper oil.

Another embodiment comprises cyclosporin A, Abil WE-09®, and Juniper oil.

Another embodiment comprises cyclosporin A, Dow Corning 1248 Fluid®, and Juniper oil.

Another embodiment comprises cyclosporin A, Dow Corning 3225C®, and Juniper oil.

Another embodiment comprises cyclosporin A, Dow Corning 5200®, and Juniper oil.

Another embodiment comprises cyclosporin A, Dow Corning Q4-3667®, and Juniper oil.

Another embodiment comprises cyclosporin A, Monasil PCA®, and Juniper oil.

Another embodiment comprises cyclosporin A, Monasil PDM®, and Juniper oil.

Another embodiment comprises cyclosporin A, Monasil PLN®, and Juniper oil.

Another embodiment comprises cyclosporin A, Polyderm PPI-SI-WS®, and Juniper oil.

Another embodiment comprises cyclosporin A, Troysol 380W®, and Juniper oil.

Another embodiment comprises cyclosporin A, Troysol S366®, and Juniper oil.

Another embodiment comprises cyclosporin A, a Sorbitan Derivative, and Juniper oil.

Another embodiment comprises cyclosporin A, Alkamuls SML®, and Juniper oil.

Another embodiment comprises cyclosporin A, Alkamuls SMO®, and Juniper oil.

Another embodiment comprises cyclosporin A, Alkamuls STO®, and Juniper oil.

Another embodiment comprises cyclosporin A, Arlacel 20®, and Juniper oil.

Another embodiment comprises cyclosporin A, Arlacel 40®, and Juniper oil.

Another embodiment comprises cyclosporin A, Arlacel 60®, and Juniper oil.

Another embodiment comprises cyclosporin A, Arlacel 80®, and Juniper oil.

Another embodiment comprises cyclosporin A, Arlacel C®, and Juniper oil.

Another embodiment comprises cyclosporin A, Armul 21®, and Juniper oil.

Another embodiment comprises cyclosporin A, Atlox 80®, and Juniper oil.

Another embodiment comprises cyclosporin A, Atlox 847®, and Juniper oil.

Another embodiment comprises cyclosporin A, Atlox 1045A®, and Juniper oil.

Another embodiment comprises cyclosporin A, Canarcel 20®, and Juniper oil.

Another embodiment comprises cyclosporin A, Canarcel 60®, and Juniper oil.

Another embodiment comprises cyclosporin A, Canarcel 80®, and Juniper oil.

Another embodiment comprises cyclosporin A, Canarcel TW 20®, and Juniper oil.

Another embodiment comprises cyclosporin A, Canarcel TW 60®, and Juniper oil.

Another embodiment comprises cyclosporin A, Canarcel TW 80®, and Juniper oil.

Another embodiment comprises cyclosporin A, Coladet BSB-P®, and Juniper oil.

Another embodiment comprises cyclosporin A, Customulse O-20®, and Juniper oil.

Another embodiment comprises cyclosporin A, Dehymuls E®, and Juniper oil.

Another embodiment comprises cyclosporin A, DeSotan SMO®, and Juniper oil.

Another embodiment comprises cyclosporin A, DeSotan SMO-20®, and Juniper oil.

Another embodiment comprises cyclosporin A, DeSotan SMT®, and Juniper oil.

Another embodiment comprises cyclosporin A, DeSotan SMT-20®, and Juniper oil.

Another embodiment comprises cyclosporin A, Durfax 60®, and Juniper oil.

Another embodiment comprises cyclosporin A, Durfax 65®, and Juniper oil.

Another embodiment comprises cyclosporin A, Durfax 80®, and Juniper oil.

Another embodiment comprises cyclosporin A, Durtan 60®, and Juniper oil.

Another embodiment comprises cyclosporin A, Durtan 65®, and Juniper oil.

Another embodiment comprises cyclosporin A, Liposorb L®, and Juniper oil.

Another embodiment comprises cyclosporin A, Liposorb L-10®, and Juniper oil.

Another embodiment comprises cyclosporin A, Liposorb L-20®, and Juniper oil.

Another embodiment comprises cyclosporin A, Liposorb O®, and Juniper oil.

Another embodiment comprises cyclosporin A, Liposorb O-20®, and Juniper oil.

Another embodiment comprises cyclosporin A, Liposorb P®, and Juniper oil.

Another embodiment comprises cyclosporin A, Liposorb P-20®, and Juniper oil.

Another embodiment comprises cyclosporin A, Liposorb S®, and Juniper oil.

Another embodiment comprises cyclosporin A, Liposorb S-20®, and Juniper oil.

Another embodiment comprises cyclosporin A, Liposorb SQO®, and Juniper oil.

Another embodiment comprises cyclosporin A, Liposorb TO®, and Juniper oil.

Another embodiment comprises cyclosporin A, Liposorb TS®, and Juniper oil.

Another embodiment comprises cyclosporin A, Liposorb TS-20®, and Juniper oil.

Another embodiment comprises cyclosporin A, Lumisorb PS®, and Juniper oil.

Another embodiment comprises cyclosporin A, Lumisorb SMO (T)®, and Juniper oil.

Another embodiment comprises cyclosporin A, Lumisorb SMS K®, and Juniper oil.

Another embodiment comprises cyclosporin A, Lumisorb SSO®, and Juniper oil.

Another embodiment comprises cyclosporin A, Lumisorb STS K®, and Juniper oil.

Another embodiment comprises cyclosporin A, Lumisorb STT®, and Juniper oil.

Another embodiment comprises cyclosporin A, Magrabar SMO®, and Juniper oil.

Another embodiment comprises cyclosporin A, Magrabar SMO-VEG®, and Juniper oil.

Another embodiment comprises cyclosporin A, Magrabar SMT®, and Juniper oil.

Another embodiment comprises cyclosporin A, Magrabar STO®, and Juniper oil.

Another embodiment comprises cyclosporin A, Miracare BC-27®, and Juniper oil.

Another embodiment comprises cyclosporin A, Ritabate 20®, and Juniper oil.

Another embodiment comprises cyclosporin A, Ritabate 40®, and Juniper oil.

Another embodiment comprises cyclosporin A, Ritabate 60®, and Juniper oil.

Another embodiment comprises cyclosporin A, Ritabate 80®, and Juniper oil.

Another embodiment comprises cyclosporin A, T-Maz®, and Juniper oil.

Another embodiment comprises cyclosporin A, Tego SML®, and Juniper oil.

Another embodiment comprises cyclosporin A, Tego SML 20®, and Juniper oil.

Another embodiment comprises cyclosporin A, Tego SMO 80 V®, and Juniper oil.

Another embodiment comprises cyclosporin A, Tego SMO V®, and Juniper oil.

Another embodiment comprises cyclosporin A, Tego SMS®, and Juniper oil.

Another embodiment comprises cyclosporin A, Tego STO V®, and Juniper oil.

Another embodiment comprises cyclosporin A, Tween 21®, and Juniper oil.

Another embodiment comprises cyclosporin A, Tween 40®, and Juniper oil.

Another embodiment comprises cyclosporin A, Tween 60®, and Juniper oil.

Another embodiment comprises cyclosporin A, Tween 60 K®, and Juniper oil.

Another embodiment comprises cyclosporin A, Tween 61®, and Juniper oil.

Another embodiment comprises cyclosporin A, Tween 65®, and Juniper oil.

Another embodiment comprises cyclosporin A, Tween 80®, and Juniper oil.

Another embodiment comprises cyclosporin A, Tween 80 K®, and Juniper oil.

Another embodiment comprises cyclosporin A, Tween 81®, and Juniper oil.

Another embodiment comprises cyclosporin A, Tween 85®, and Juniper oil.

Another embodiment comprises cyclosporin A, a Sucrose or Glucose Ester, or Derivative thereof, and Juniper oil.

Another embodiment comprises cyclosporin A, DeSulf GOS-P-60WCG®, and Juniper oil.

Another embodiment comprises cyclosporin A, Glucam E-20 Distearate®, and Juniper oil.

Another embodiment comprises cyclosporin A, Glucamate DOE-120®, and Juniper oil.

Another embodiment comprises cyclosporin A, Glucamate SSE-20®, and Juniper oil.

Another embodiment comprises cyclosporin A, Glucate DO®, and Juniper oil.

Another embodiment comprises cyclosporin A, Glucate SS®, and Juniper oil.

Another embodiment comprises cyclosporin A, Glucopon 425 UP®, and Juniper oil.

Another embodiment comprises cyclosporin A, Isolan IS®, and Juniper oil.

Another embodiment comprises cyclosporin A, Mazon 40®, and Juniper oil.

Another embodiment comprises cyclosporin A, Montanov 82®, and Juniper oil.

Another embodiment comprises cyclosporin A, Montanov 202®, and Juniper oil.

Another embodiment comprises cyclosporin A, Montanov S®, and Juniper oil.

Another embodiment comprises cyclosporin A, Rheozan®, and Juniper oil.

Another embodiment comprises cyclosporin A, Simulsol AS 48®, and Juniper oil.

Another embodiment comprises cyclosporin A, Simulsol SL 4®, and Juniper oil.

Another embodiment comprises cyclosporin A, Simulsol SL 10®, and Juniper oil.

Another embodiment comprises cyclosporin A, Simulsol SL 11W®, and Juniper oil.

Another embodiment comprises cyclosporin A, Simulsol SL 55®, and Juniper oil.

Another embodiment comprises cyclosporin A, Suga Nate 100 and 160®, and Juniper oil.

Another embodiment comprises cyclosporin A, Tego Care 450®, and Juniper oil.

Another embodiment comprises cyclosporin A, Tego Care CG 90®, and Juniper oil.

Another embodiment comprises cyclosporin A, Tego Care PS®, and Juniper oil.

Another embodiment comprises cyclosporin A, Tegosoft PSE 141 G®, and Juniper oil.

Another embodiment comprises cyclosporin A, Tegotens G 826®, and Juniper oil.

Another embodiment comprises cyclosporin A, Triton BG-10 (70%)®, and Juniper oil.

Another embodiment comprises cyclosporin A, Triton CG-110 (60%)®, and Juniper oil.

Another embodiment comprises cyclosporin A, Wickenol 545®, and Juniper oil.

Another embodiment comprises cyclosporin A, an alcohol, and Lemon oil.

Another embodiment comprises cyclosporin A, Diglycerol®, and Lemon oil.

Another embodiment comprises cyclosporin A, Hetoxide GT-80®, and Lemon oil.

Another embodiment comprises cyclosporin A, Lexemul BEO®, and Lemon oil.

Another embodiment comprises cyclosporin A, Polyglycerol-3®, and Lemon oil.

Another embodiment comprises cyclosporin A, Redicote E Series®, and Lemon oil.

Another embodiment comprises cyclosporin A, Simulsol OX 1005L®, and Lemon oil.

Another embodiment comprises cyclosporin A, Stanfax 567®, and Lemon oil.

Another embodiment comprises cyclosporin A, TA-1618®, and Lemon oil.

Another embodiment comprises cyclosporin A, Witconol H-31 A®, and Lemon oil.

Another embodiment comprises cyclosporin A, an amine oxide, and Lemon oil.

Another embodiment comprises cyclosporin A, AO-405®, and Lemon oil.

Another embodiment comprises cyclosporin A, AO-455®, and Lemon oil.

Another embodiment comprises cyclosporin A, AO 728 Special®, and Lemon oil.

Another embodiment comprises cyclosporin A, Barlox 12®, and Lemon oil.

Another embodiment comprises cyclosporin A, Barlox 14®, and Lemon oil.

Another embodiment comprises cyclosporin A, Burcoxide Lo®, and Lemon oil.

Another embodiment comprises cyclosporin A, Caloxamine LO®, and Lemon oil.

Another embodiment comprises cyclosporin A, Chemoxide CAW®, and Lemon oil.

Another embodiment comprises cyclosporin A, Chemoxide LM-30®, and Lemon oil.

Another embodiment comprises cyclosporin A, Chemoxide LO®, and Lemon oil.

Another embodiment comprises cyclosporin A, Chemoxide MO®, and Lemon oil.

Another embodiment comprises cyclosporin A, Colalux CAO-35®, and Lemon oil.

Another embodiment comprises cyclosporin A, Colalux LO®, and Lemon oil.

Another embodiment comprises cyclosporin A, DeMox CAPO®, and Lemon oil.

Another embodiment comprises cyclosporin A, DeMox CSG-30n, and Lemon oil.

Another embodiment comprises cyclosporin A, DeMox LAO®, and Lemon oil.

Another embodiment comprises cyclosporin A, Emcol L®, and Lemon oil.

Another embodiment comprises cyclosporin A, Empigen OB®, and Lemon oil.

Another embodiment comprises cyclosporin A, Empigen OS/A®, and Lemon oil.

Another embodiment comprises cyclosporin A, Foamox CDO®, and Lemon oil.

Another embodiment comprises cyclosporin A, Foamox DMM®, and Lemon oil.

Another embodiment comprises cyclosporin A, Foamox DMS®, and Lemon oil.

Another embodiment comprises cyclosporin A, Genaminox KC®, and Lemon oil.

Another embodiment comprises cyclosporin A, Genaminox LA®, and Lemon oil.

Another embodiment comprises cyclosporin A, Hartofoam SAO®, and Lemon oil.

Another embodiment comprises cyclosporin A, Hartox DMCD®, and Lemon oil.

Another embodiment comprises cyclosporin A, Lipowax DAT®, and Lemon oil.

Another embodiment comprises cyclosporin A, Lipowax PB Pastilles®, and Lemon oil.

Another embodiment comprises cyclosporin A, Mackamine C8®, and Lemon oil.

Another embodiment comprises cyclosporin A, Mackamine C10®, and Lemon oil.

Another embodiment comprises cyclosporin A, Mackamine C14®, and Lemon oil.

Another embodiment comprises cyclosporin A, Mackamine CAO®, and Lemon oil.

Another embodiment comprises cyclosporin A, Mackamine CO®, and Lemon oil.

Another embodiment comprises cyclosporin A, Mackamine LO®, and Lemon oil.

Another embodiment comprises cyclosporin A, Mackamine O2®, and Lemon oil.

Another embodiment comprises cyclosporin A, Mackamine SAO®, and Lemon oil.

Another embodiment comprises cyclosporin A, Mackamine SO®, and Lemon oil.

Another embodiment comprises cyclosporin A, Mazox KCAO®, and Lemon oil.

Another embodiment comprises cyclosporin A, Monalac MO®, and Lemon oil.

Another embodiment comprises cyclosporin A, Norfox LDA®, and Lemon oil.

Another embodiment comprises cyclosporin A, Rhodamox LO®, and Lemon oil.

Another embodiment comprises cyclosporin A, Schercamox C-AA®, and Lemon oil.

Another embodiment comprises cyclosporin A, Schercamox DMA®, and Lemon oil.

Another embodiment comprises cyclosporin A, Schercamox DML®, and Lemon oil.

Another embodiment comprises cyclosporin A, Schercamox DMM®, and Lemon oil.

Another embodiment comprises cyclosporin A, Schercamox DMS®, and Lemon oil.

Another embodiment comprises cyclosporin A, Tegotens DO®, and Lemon oil.

Another embodiment comprises cyclosporin A, Tomah AO-14-2®, and Lemon oil.

Another embodiment comprises cyclosporin A, Triaminox CDO®, and Lemon oil.

Another embodiment comprises cyclosporin A, a block polymer, and Lemon oil.

Another embodiment comprises cyclosporin A, AL 2070®, and Lemon oil.

Another embodiment comprises cyclosporin A, Antarox 17-R-2®, and Lemon oil.

Another embodiment comprises cyclosporin A, Antarox 25-R-2®, and Lemon oil.

Another embodiment comprises cyclosporin A, Antarox 31-R-1®, and Lemon oil.

Another embodiment comprises cyclosporin A, Antarox P-84®, and Lemon oil.

Another embodiment comprises cyclosporin A, Antarox P-104/H®, and Lemon oil.

Another embodiment comprises cyclosporin A, Arnox BP-Series®, and Lemon oil.

Another embodiment comprises cyclosporin A, Chemonic 435®, and Lemon oil.

Another embodiment comprises cyclosporin A, Chemonic D-25®, and Lemon oil.

Another embodiment comprises cyclosporin A, Chemonic PL Series®, and Lemon oil.

Another embodiment comprises cyclosporin A, Ethox L-121®, and Lemon oil.

Another embodiment comprises cyclosporin A, Ethox L-122®, and Lemon oil.

Another embodiment comprises cyclosporin A, Genapol PF-10®, and Lemon oil.

Another embodiment comprises cyclosporin A, Genapol PF-20®, and Lemon oil.

Another embodiment comprises cyclosporin A, Genapol PF-40A®, and Lemon oil.

Another embodiment comprises cyclosporin A, Norfox 2-LF®, and Lemon oil.

Another embodiment comprises cyclosporin A, Simulsol NW 342®, and Lemon oil.

Another embodiment comprises cyclosporin A, T-Det BP-1®, and Lemon oil.

Another embodiment comprises cyclosporin A, T-Det XD®, and Lemon oil.

Another embodiment comprises cyclosporin A, T-Det XH®, and Lemon oil.

Another embodiment comprises cyclosporin A, Triton CF-32®, and Lemon oil.

Another embodiment comprises cyclosporin A, Witconol 171®, and Lemon oil.

Another embodiment comprises cyclosporin A, Witconol 324®, and Lemon oil.

Another embodiment comprises cyclosporin A, Witconol 324D®, and Lemon oil.

Another embodiment comprises cyclosporin A, Witconol PD-2000®, and Lemon oil.

Another embodiment comprises cyclosporin A, a Carboxylated Alcohoi or Alkylphenol Ethoxylate, and Lemon oil.

Another embodiment comprises cyclosporin A, Emcol CN-6®, and Lemon oil.

Another embodiment comprises cyclosporin A, Ethcarb®, and Lemon oil.

Another embodiment comprises cyclosporin A, Gemtex WNT-Conc®, and Lemon oil.

Another embodiment comprises cyclosporin A, Incrodet TD7-C®, and Lemon oil.

Another embodiment comprises cyclosporin A, Marlinat CM 105/80®, and Lemon oil.

Another embodiment comprises cyclosporin A, Marlowet 1072®, and Lemon oil.

Another embodiment comprises cyclosporin A, Marlowet 4530®, and Lemon oil.

Another embodiment comprises cyclosporin A, Marlowet 4530 LF®, and Lemon oil.

Another embodiment comprises cyclosporin A, Marlowet 4534®, and Lemon oil.

Another embodiment comprises cyclosporin A, Marlowet 4538®, and Lemon oil.

Another embodiment comprises cyclosporin A, Marlowet 4539®, and Lemon oil.

Another embodiment comprises cyclosporin A, Marlowet 4539 LF®, and Lemon oil.

Another embodiment comprises cyclosporin A, Marlowet 4541®, and Lemon oil.

Another embodiment comprises cyclosporin A, Miranate LEC-80®, and Lemon oil.

Another embodiment comprises cyclosporin A, Sandopan B®, and Lemon oil.

Another embodiment comprises cyclosporin A, Sandopan B Modified®, and Lemon oil.

Another embodiment comprises cyclosporin A, Sandopan LS-24 Gel®, and Lemon oil.

Another embodiment comprises cyclosporin A, Surfine T-A®, and Lemon oil.

Another embodiment comprises cyclosporin A, a Carboxylic Acid/Fatty Acid, and Lemon oil.

Another embodiment comprises cyclosporin A, Colaterge RAM®, and Lemon oil.

Another embodiment comprises cyclosporin A, Colatrope INC®, and Lemon oil.

Another embodiment comprises cyclosporin A, Crodacid B®, and Lemon oil.

Another embodiment comprises cyclosporin A, DeTrope CA-100®, and Lemon oil.

Another embodiment comprises cyclosporin A, Latol MTO®, and Lemon oil.

Another embodiment comprises cyclosporin A, Lumulse CC-33 K®, and Lemon oil.

Another embodiment comprises cyclosporin A, Mulls 2218®, and Lemon oil.

Another embodiment comprises cyclosporin A, OL-600®, and Lemon oil.

Another embodiment comprises cyclosporin A, OL-800®, and Lemon oil.

Another embodiment comprises cyclosporin A, R-910®, and Lemon oil.

Another embodiment comprises cyclosporin A, S-210®, and Lemon oil.

Another embodiment comprises cyclosporin A, Sandopan DTC Acid®, and Lemon oil.

Another embodiment comprises cyclosporin A, Sandopan LS 24 N®, and Lemon oil.

Another embodiment comprises cyclosporin A, Sandopan MA-18®, and Lemon oil.

Another embodiment comprises cyclosporin A, an Ethoxylated Alcohol, and Lemon oil.

Another embodiment comprises cyclosporin A, Adsee 799®, and Lemon oil.

Another embodiment comprises cyclosporin A, Adsee 799®, and Lemon oil.

Another embodiment comprises cyclosporin A, Alfonic 610-3.5®, and Lemon oil.

Another embodiment comprises cyclosporin A, Alfonic 810-2®, and Lemon oil.

Another embodiment comprises cyclosporin A, Alfonic 810-6®, and Lemon oil.

Another embodiment comprises cyclosporin A, Alfonic 1012-3®, and Lemon oil.

Another embodiment comprises cyclosporin A, Alfonic 1012-5®, and Lemon oil.

Another embodiment comprises cyclosporin A, Alfonic 1216CO-1.5®, and Lemon oil.

Another embodiment comprises cyclosporin A, Alfonic 1216CO-7®, and Lemon oil.

Another embodiment comprises cyclosporin A, Alfonic 1412-3®, and Lemon oil.

Another embodiment comprises cyclosporin A, Alfonic 1412-7®, and Lemon oil.

Another embodiment comprises cyclosporin A, Arlasolve 200®, and Lemon oil.

Another embodiment comprises cyclosporin A, Arlasolve 200 Liquid®, and Lemon oil.

Another embodiment comprises cyclosporin A, Armix 180-C®, and Lemon oil.

Another embodiment comprises cyclosporin A, Armix 183®, and Lemon oil.

Another embodiment comprises cyclosporin A, Armul 2404®, and Lemon oil.

Another embodiment comprises cyclosporin A, Armul 2404®, and Lemon oil.

Another embodiment comprises cyclosporin A, Atlas EMJ-C®, and Lemon oil.

Another embodiment comprises cyclosporin A, Atlas G-2109®, and Lemon oil.

Another embodiment comprises cyclosporin A, Atlas G-3886®, and Lemon oil.

Another embodiment comprises cyclosporin A, Atlas G-3890®, and Lemon oil.

Another embodiment comprises cyclosporin A, Bio Soft E-200®, and Lemon oil.

Another embodiment comprises cyclosporin A, Bio Soft E-300®, and Lemon oil.

Another embodiment comprises cyclosporin A, Bio Soft E-400®, and Lemon oil.

Another embodiment comprises cyclosporin A, Bio Soft EN 600®, and Lemon oil.

Another embodiment comprises cyclosporin A, Bio Soft TD-400®, and Lemon oil.

Another embodiment comprises cyclosporin A, Bio Soft TD-630®, and Lemon oil.

Another embodiment comprises cyclosporin A, Brij 30®, and Lemon oil.

Another embodiment comprises cyclosporin A, Brij 52®, and Lemon oil.

Another embodiment comprises cyclosporin A, Brij 56®, and Lemon oil.

Another embodiment comprises cyclosporin A, Brij 58®, and Lemon oil.

Another embodiment comprises cyclosporin A, Brij 72®, and Lemon oil.

Another embodiment comprises cyclosporin A, Brij 76®, and Lemon oil.

Another embodiment comprises cyclosporin A, Brij 78®, and Lemon oil.

Another embodiment comprises cyclosporin A, Brij 93®, and Lemon oil.

Another embodiment comprises cyclosporin A, Brij 97®, and Lemon oil.

Another embodiment comprises cyclosporin A, Brij 98®, and Lemon oil.

Another embodiment comprises cyclosporin A, Brij 700®, and Lemon oil.

Another embodiment comprises cyclosporin A, Brij 700 S®, and Lemon oil.

Another embodiment comprises cyclosporin A, Brij 721®, and Lemon oil.

Another embodiment comprises cyclosporin A, Brij 721 S®, and Lemon oil.

Another embodiment comprises cyclosporin A, Burcoterge CDG®, and Lemon oil.

Another embodiment comprises cyclosporin A, Canasol AT 600®, and Lemon oil.

Another embodiment comprises cyclosporin A, Canasol AT 800®, and Lemon oil.

Another embodiment comprises cyclosporin A, Canasol AT 1200®, and Lemon oil.

Another embodiment comprises cyclosporin A, Canasol BJ 35®, and Lemon oil.

Another embodiment comprises cyclosporin A, Canasol BJ 36®, and Lemon oil.

Another embodiment comprises cyclosporin A, Canasol BJ 52®, and Lemon oil.

Another embodiment comprises cyclosporin A, Canasol BJ 58®, and Lemon oil.

Another embodiment comprises cyclosporin A, Canasol BJ 72®, and Lemon oil.

Another embodiment comprises cyclosporin A, Canasol BJ 78®, and Lemon oil.

Another embodiment comprises cyclosporin A, Canasol BJ 98®, and Lemon oil.

Another embodiment comprises cyclosporin A, Canasol BJ 307®, and Lemon oil.

Another embodiment comprises cyclosporin A, Cerfak 1400®, and Lemon oil.

Another embodiment comprises cyclosporin A, Cetomacrogol 1000 BP®, and Lemon oil.

Another embodiment comprises cyclosporin A, Chemonic C-2®, and Lemon oil.

Another embodiment comprises cyclosporin A, Chemonic C-10®, and Lemon oil.

Another embodiment comprises cyclosporin A, Chemonic C-20®, and Lemon oil.

Another embodiment comprises cyclosporin A, Chemonic CT-12®, and Lemon oil.

Another embodiment comprises cyclosporin A, Chemonic CT-20®, and Lemon oil.

Another embodiment comprises cyclosporin A, Chemonic CT-30®, and Lemon oil.

Another embodiment comprises cyclosporin A, Chemonic CT-55®, and Lemon oil.

Another embodiment comprises cyclosporin A, Chemonic G-7®, and Lemon oil.

Another embodiment comprises cyclosporin A, Chemonic G-26®, and Lemon oil.

Another embodiment comprises cyclosporin A, Chemonic L-4®, and Lemon oil.

Another embodiment comprises cyclosporin A, Chemonic L-7®, and Lemon oil.

Another embodiment comprises cyclosporin A, Chemonic L-12®, and Lemon oil.

Another embodiment comprises cyclosporin A, Chemonic L-23®, and Lemon oil.

Another embodiment comprises cyclosporin A, Chemonic O-2®, and Lemon oil.

Another embodiment comprises cyclosporin A, Chembnic O-5®, and Lemon oil.

Another embodiment comprises cyclosporin A, Chemonic O-10®, and Lemon oil.

Another embodiment comprises cyclosporin A, Chemonic O-20®, and Lemon oil.

Another embodiment comprises cyclosporin A, Chemonic S-2®, and Lemon oil.

Another embodiment comprises cyclosporin A, Chemonic S-10®, and Lemon oil.

Another embodiment comprises cyclosporin A, Chemonic S-20®, and Lemon oil.

Another embodiment comprises cyclosporin A, Colamulse FE®, and Lemon oil.

Another embodiment comprises cyclosporin A, Cremophor A 20®, and Lemon oil.

Another embodiment comprises cyclosporin A, Cremophor SA 2®, and Lemon oil.

Another embodiment comprises cyclosporin A, Dehydol 100®, and Lemon oil.

Another embodiment comprises cyclosporin A, Dehydol O-4®, and Lemon oil.

Another embodiment comprises cyclosporin A, Delonic C-18®, and Lemon oil.

Another embodiment comprises cyclosporin A, DeSonic 6T®, and Lemon oil.

Another embodiment comprises cyclosporin A, DeSonic 9D®, and Lemon oil.

Another embodiment comprises cyclosporin A, DeSonic 9T®, and Lemon oil.

Another embodiment comprises cyclosporin A, DeSonic 12D®, and Lemon oil.

Another embodiment comprises cyclosporin A, DeSonic 12T®, and Lemon oil.

Another embodiment comprises cyclosporin A, DeSonic 15T®, and Lemon oil.

Another embodiment comprises cyclosporin A, DeSonic TDA-9®, and Lemon oil.

Another embodiment comprises cyclosporin A, DeThox GLG-7®, and Lemon oil.

Another embodiment comprises cyclosporin A, DeThox GLG-26®, and Lemon oil.

Another embodiment comprises cyclosporin A, DeThox LA-4®, and Lemon oil.

Another embodiment comprises cyclosporin A, DeThox LA-23®, and Lemon oil.

Another embodiment comprises cyclosporin A, DeThox SA-80®, and Lemon oil.

Another embodiment comprises cyclosporin A, Disponil O5®, and Lemon oil.

Another embodiment comprises cyclosporin A, Eccoterge EO-41 B®, and Lemon oil.

Another embodiment comprises cyclosporin A, Empilan KA2.5/90®, and Lemon oil.

Another embodiment comprises cyclosporin A, Empilan KA5/90®, and Lemon oil.

Another embodiment comprises cyclosporin A, Empilan KM-20®, and Lemon oil.

Another embodiment comprises cyclosporin A, Empilan KM-50®, and Lemon oil.

Another embodiment comprises cyclosporin A, Empilan L-23®, and Lemon oil.

Another embodiment comprises cyclosporin A, Ethylan 25-3®, and Lemon oil.

Another embodiment comprises cyclosporin A, Ethylan 1204®, and Lemon oil.

Another embodiment comprises cyclosporin A, Ethylan DA-4®, and Lemon oil.

Another embodiment comprises cyclosporin A, Ethylan LA-230®, and Lemon oil.

Another embodiment comprises cyclosporin A, Ethylan SN®, and Lemon oil.

Another embodiment comprises cyclosporin A, Ethylan TD-60®, and Lemon oil.

Another embodiment comprises cyclosporin A, Ethylan TD-100®, and Lemon oil.

Another embodiment comprises cyclosporin A, Ethylan TD-1407®, and Lemon oil.

Another embodiment comprises cyclosporin A, Eumulgin B1®, and Lemon oil.

Another embodiment comprises cyclosporin A, Eumulgin B2®, and Lemon oil.

Another embodiment comprises cyclosporin A, Eumulgin B3®, and Lemon oil.

Another embodiment comprises cyclosporin A, Eumulgin O-10®, and Lemon oil.

Another embodiment comprises cyclosporin A, Flo Mo 80/20®, and Lemon oil.

Another embodiment comprises cyclosporin A, Flo Mo Low Foam®, and Lemon oil.

Another embodiment comprises cyclosporin A, Forlan C-24®, and Lemon oil.

Another embodiment comprises cyclosporin A, Genapol 1454®, and Lemon oil.

Another embodiment comprises cyclosporin A, Genapol BA-020®, and Lemon oil.

Another embodiment comprises cyclosporin A, Genapol BA-040®, and Lemon oil.

Another embodiment comprises cyclosporin A, Genapol C-100®, and Lemon oil.

Another embodiment comprises cyclosporin A, Genapol DA 060®, and Lemon oil.

Another embodiment comprises cyclosporin A, Genapol HS 020®, and Lemon oil.

Another embodiment comprises cyclosporin A, Genapol HS 200®, and Lemon oil.

Another embodiment comprises cyclosporin A, Genapol ID-040®, and Lemon oil.

Another embodiment comprises cyclosporin A, Genapol ID-060®, and Lemon oil.

Another embodiment comprises cyclosporin A, Genapol ID-090®, and Lemon oil.

Another embodiment comprises cyclosporin A, Genapol LA 010®, and Lemon oil.

Another embodiment comprises cyclosporin A, Genapol LA 020®, and Lemon oil.

Another embodiment comprises cyclosporin A, Genapol LA 030®, and Lemon oil.

Another embodiment comprises cyclosporin A, Genapol LA 040®, and Lemon oil.

Another embodiment comprises cyclosporin A, Genapol LA 050®, and Lemon oil.

Another embodiment comprises cyclosporin A, Genapol LA 060®, and Lemon oil.

Another embodiment comprises cyclosporin A, Genapol LA 070®, and Lemon oil.

Another embodiment comprises cyclosporin A, Genapol LA 070S®, and Lemon oil.

Another embodiment comprises cyclosporin A, Genapol LA 230®, and Lemon oil.

Another embodiment comprises cyclosporin A, Genapol O 020®, and Lemon oil.

Another embodiment comprises cyclosporin A, Genapol O 050®, and Lemon oil.

Another embodiment comprises cyclosporin A, Genapol O 100®, and Lemon oil.

Another embodiment comprises cyclosporin A, Genapol O 200®, and Lemon oil.

Another embodiment comprises cyclosporin A, Genapol SA 030®, and Lemon oil.

Another embodiment comprises cyclosporin A, Genapol SA 120®, and Lemon oil.

Another embodiment comprises cyclosporin A, Genapol T-020®, and Lemon oil.

Another embodiment comprises cyclosporin A, Genapol UD-030®, and Lemon oil.

Another embodiment comprises cyclosporin A, Genapol UD-050®, and Lemon oil.

Another embodiment comprises cyclosporin A, Genapol UD-070®, and Lemon oil.

Another embodiment comprises cyclosporin A, Genapol UD-079®, and Lemon oil.

Another embodiment comprises cyclosporin A, Genapol UD-080®, and Lemon oil.

Another embodiment comprises cyclosporin A, Genapol UD-110®, and Lemon oil.

Another embodiment comprises cyclosporin A, Genapol X 030®, and Lemon oil.

Another embodiment comprises cyclosporin A, Genapol X 050®, and Lemon oil.

Another embodiment comprises cyclosporin A, Genapol X 060®, and Lemon oil.

Another embodiment comprises cyclosporin A, Genapol X 070®, and Lemon oil.

Another embodiment comprises cyclosporin A, Genapol X 080®, and Lemon oil.

Another embodiment comprises cyclosporin A, Genapol X 100®, and Lemon oil.

Another embodiment comprises cyclosporin A, Genapol X159®, and Lemon oil.

Another embodiment comprises cyclosporin A, Generol 122 E5®, and Lemon oil.

Another embodiment comprises cyclosporin A, Generol 122 E25®, and Lemon oil.

Another embodiment comprises cyclosporin A, Hostacerin T-3®, and Lemon oil.

Another embodiment comprises cyclosporin A, Iconol LF 110 ®, and Lemon oil.

Another embodiment comprises cyclosporin A, Incropol CS-20®, and Lemon oil.

Another embodiment comprises cyclosporin A, Lexemul CS-20®, and Lemon oil.

Another embodiment comprises cyclosporin A, Liponic EG-1®, and Lemon oil.

Another embodiment comprises cyclosporin A, Lipowax D®, and Lemon oil.

Another embodiment comprises cyclosporin A, Lipowax G®, and Lemon oil.

Another embodiment comprises cyclosporin A, Lipowax NI®, and Lemon oil.

Another embodiment comprises cyclosporin A, Lipowax P®, and Lemon oil.

Another embodiment comprises cyclosporin A, Lipowax P-31®, and Lemon oil.

Another embodiment comprises cyclosporin A, Lipowax PR®, and Lemon oil.

Another embodiment comprises cyclosporin A, Lumulse CS-20®, and Lemon oil.

Another embodiment comprises cyclosporin A, Macol CSA-20®, and Lemon oil.

Another embodiment comprises cyclosporin A, Marlox B 24/50®, and Lemon oil.

Another embodiment comprises cyclosporin A, Mazawet 77®, and Lemon oil.

Another embodiment comprises cyclosporin A, Norfox 1713®, and Lemon oil.

Another embodiment comprises cyclosporin A, Norfox 2579®, and Lemon oil.

Another embodiment comprises cyclosporin A, Norfox Lo Foam®, and Lemon oil.

Another embodiment comprises cyclosporin A, Promulgen D®, and Lemon oil.

Another embodiment comprises cyclosporin A, Promulgen G®, and Lemon oil.

Another embodiment comprises cyclosporin A, Renex 30®, and Lemon oil.

Another embodiment comprises cyclosporin A, Renex 36®, and Lemon oil.

Another embodiment comprises cyclosporin A, Rhodasurf A 24®, and Lemon oil.

Another embodiment comprises cyclosporin A, Rhodasurf AAE-10®, and Lemon oil.

Another embodiment comprises cyclosporin A, Rhodasurf BEH-25®, and Lemon oil.

Another embodiment comprises cyclosporin A, Rhodasurf BEH-40®, and Lemon oil.

Another embodiment comprises cyclosporin A, Rhodasurf DA 530®, and Lemon oil.

Another embodiment comprises cyclosporin A, Rhodasurf DA 630®, and Lemon oil.

Another embodiment comprises cyclosporin A, Rhodasurf DA 639®, and Lemon oil.

Another embodiment comprises cyclosporin A, Rhodasurf LAN-23®, and Lemon oil.

Another embodiment comprises cyclosporin A, Rhodasurf ON-870®, and Lemon oil.

Another embodiment comprises cyclosporin A, Rhodasurf ON-877®, and Lemon oil.

Another embodiment comprises cyclosporin A, Rhodasurf TB-970 FLK®, and Lemon oil.

Another embodiment comprises cyclosporin A, Ritacet-20®, and Lemon oil.

Another embodiment comprises cyclosporin A, Ritachol 1000®, and Lemon oil.

Another embodiment comprises cyclosporin A, Ritachol 2000®, and Lemon oil.

Another embodiment comprises cyclosporin A, Ritachol 5000®, and Lemon oil.

Another embodiment comprises cyclosporin A, Ritox 35®, and Lemon oil.

Another embodiment comprises cyclosporin A, Surfonic DA-4®, and Lemon oil.

Another embodiment comprises cyclosporin A, Surfonic DA-6®, and Lemon oil.

Another embodiment comprises cyclosporin A, Surfonic L46-7®, and Lemon oil.

Another embodiment comprises cyclosporin A, Surfonic POA®, and Lemon oil.

Another embodiment comprises cyclosporin A, Synthrapol KB®, and Lemon oil.

Another embodiment comprises cyclosporin A, Teginacid®, and Lemon oil.

Another embodiment comprises cyclosporin A, Teginacid C®, and Lemon oil.

Another embodiment comprises cyclosporin A, Tegotens EC 11®, and Lemon oil.

Another embodiment comprises cyclosporin A, Tinegal NA®, and Lemon oil.

Another embodiment comprises cyclosporin A, Tomadol 400®, and Lemon oil.

Another embodiment comprises cyclosporin A, Tomadol 600®, and Lemon oil.

Another embodiment comprises cyclosporin A, Tomadol 900®, and Lemon oil.

Another embodiment comprises cyclosporin A, Uniperol O®, and Lemon oil.

Another embodiment comprises cyclosporin A, Witconol SN Series®, and Lemon oil.

Another embodiment comprises cyclosporin A, an Ethoxylated Alkylphenol, and Lemon oil.

Another embodiment comprises cyclosporin A, Antarox LF-222®, and Lemon oil.

Another embodiment comprises cyclosporin A, Atlox 775®, and Lemon oil.

Another embodiment comprises cyclosporin A, Caloxylate N-9®, and Lemon oil.

Another embodiment comprises cyclosporin A, Canasol NF-1000®, and Lemon oil.

Another embodiment comprises cyclosporin A, Canasol NF-3000®, and Lemon oil.

Another embodiment comprises cyclosporin A, Canasol NF-3070®, and Lemon oil.

Another embodiment comprises cyclosporin A, Canasol OF 1670®, and Lemon oil.

Another embodiment comprises cyclosporin A, Canasol OF 2570®, and Lemon oil.

Another embodiment comprises cyclosporin A, Canasol OF 4070®, and Lemon oil.

Another embodiment comprises cyclosporin A, Chemax DNP-8®, and Lemon oil.

Another embodiment comprises cyclosporin A, Chemax DNP-18®, and Lemon oil.

Another embodiment comprises cyclosporin A, Chemax DNP-150/50®, and Lemon oil.

Another embodiment comprises cyclosporin A, DeSonic 1.5N®, and Lemon oil.

Another embodiment comprises cyclosporin A, DeSonic 4N®, and Lemon oil.

Another embodiment comprises cyclosporin A, DeSonic 5N®, and Lemon oil.

Another embodiment comprises cyclosporin A, DeSonic 6D®, and Lemon oil.

Another embodiment comprises cyclosporin A, DeSonic 6N®, and Lemon oil.

Another embodiment comprises cyclosporin A, DeSonic 7N®, and Lemon oil.

Another embodiment comprises cyclosporin A, DeSonic 9N®, and Lemon oil.

Another embodiment comprises cyclosporin A, DeSonic 10D®, and Lemon oil.

Another embodiment comprises cyclosporin A, DeSonic 11N®, and Lemon oil.

Another embodiment comprises cyclosporin A, DeSonic 12N®, and Lemon oil.

Another embodiment comprises cyclosporin A, DeSonic 15N®, and Lemon oil.

Another embodiment comprises cyclosporin A, DeSonic 20N®, and Lemon oil.

Another embodiment comprises cyclosporin A, Eccoscour RC®, and Lemon oil.

Another embodiment comprises cyclosporin A, Eccoterge EO-100®, and Lemon oil.

Another embodiment comprises cyclosporin A, Emulsifier 632/90%®, and Lemon oil.

Another embodiment comprises cyclosporin A, Geronol AG-821®, and Lemon oil.

Another embodiment comprises cyclosporin A, Gradonic N-95®, and Lemon oil.

Another embodiment comprises cyclosporin A, Hetoxide NP-4®, and Lemon oil.

Another embodiment comprises cyclosporin A, Hetoxide NP-30®, and Lemon oil.

Another embodiment comprises cyclosporin A, Hostapal N-100®, and Lemon oil.

Another embodiment comprises cyclosporin A, Hostapal N-110®, and Lemon oil.

Another embodiment comprises cyclosporin A, Igepal CTA-639W®, and Lemon oil.

Another embodiment comprises cyclosporin A, Igepal DAP-9®, and Lemon oil.

Another embodiment comprises cyclosporin A, Igepal OD-410®, and Lemon oil.

Another embodiment comprises cyclosporin A, Igepal SS-837®, and Lemon oil.

Another embodiment comprises cyclosporin A, Lipocol NP-9 USP®, and Lemon oil.

Another embodiment comprises cyclosporin A, Macol DNP-10®, and Lemon oil.

Another embodiment comprises cyclosporin A, Marlophen NP 5®, and Lemon oil.

Another embodiment comprises cyclosporin A, Marlophen P 1®, and Lemon oil.

Another embodiment comprises cyclosporin A, Surfonic NB®, and Lemon oil.

Another embodiment comprises cyclosporin A, Surfonic OPB-307®, and Lemon oil.

Another embodiment comprises cyclosporin A, Surfonic OPB-407®, and Lemon oil.

Another embodiment comprises cyclosporin A, Syn Fac 334®, and Lemon oil.

Another embodiment comprises cyclosporin A, Syn Fac 8216®, and Lemon oil.

Another embodiment comprises cyclosporin A, Triton N-57®, and Lemon oil.

Another embodiment comprises cyclosporin A, Trycol 6956®, and Lemon oil.

Another embodiment comprises cyclosporin A, Trycol 6961®, and Lemon oil.

Another embodiment comprises cyclosporin A, Trycol 6964®, and Lemon oil.

Another embodiment comprises cyclosporin A, Trycol 6969®, and Lemon oil.

Another embodiment comprises cyclosporin A, Trycol 6974®, and Lemon oil.

Another embodiment comprises cyclosporin A, Witbreak DRB-127®, and Lemon oil.

Another embodiment comprises cyclosporin A, Witbreak DRB-127®, and Lemon oil.

Another embodiment comprises cyclosporin A, Witconol NP Series®, and Lemon oil.

Another embodiment comprises cyclosporin A, an Ethoxylated Aryl Phenol, and Lemon oil.

Another embodiment comprises cyclosporin A, Soprophor BSU®, and Lemon oil.

Another embodiment comprises cyclosporin A, Soprophor CY/8®, and Lemon oil.

Another embodiment comprises cyclosporin A, Soprophor S/25®, and Lemon oil.

Another embodiment comprises cyclosporin A, Witconol NIO®, and Lemon oil.

Another embodiment comprises cyclosporin A, Witconol NIW®, and Lemon oil.

Another embodiment comprises cyclosporin A, Witconol S-100®, and Lemon oil.

Another embodiment comprises cyclosporin A, an Ethoxylated Fatty Acid, and Lemon oil.

Another embodiment comprises cyclosporin A, Aldo PGHMS®, and Lemon oil.

Another embodiment comprises cyclosporin A, Alkamuls TO-15/HR®, and Lemon oil.

Another embodiment comprises cyclosporin A, Armotan AL-69-66®, and Lemon oil.

Another embodiment comprises cyclosporin A, Cerasynt 840®, and Lemon oil.

Another embodiment comprises cyclosporin A, Cerasynt 945®, and Lemon oil.

Another embodiment comprises cyclosporin A, Crystal Inhibitor No: 5®, and Lemon oil.

Another embodiment comprises cyclosporin A, DeThox Acid L-9®, and Lemon oil.

Another embodiment comprises cyclosporin A, DeThox Acid S-8®, and Lemon oil.

Another embodiment comprises cyclosporin A, Ethofat 242/25®, and Lemon oil.

Another embodiment comprises cyclosporin A, Hydropalat 65®, and Lemon oil.

Another embodiment comprises cyclosporin A, Lipo EGMS®, and Lemon oil.

Another embodiment comprises cyclosporin A, Lipopeg 2 DL®, and Lemon oil.

Another embodiment comprises cyclosporin A, Lipopeg 4 DL®, and Lemon oil.

Another embodiment comprises cyclosporin A, Lipopeg 4-L®, and Lemon oil.

Another embodiment comprises cyclosporin A, Lipopeg 39-S®, and Lemon oil.

Another embodiment comprises cyclosporin A, Lipopeg 4-S®, and Lemon oil.

Another embodiment comprises cyclosporin A, Lipopeg 10-S®, and Lemon oil.

Another embodiment comprises cyclosporin A, Lipopeg 100-S®, and Lemon oil.

Another embodiment comprises cyclosporin A, Lipopeg 6000 DS®, and Lemon oil.

Another embodiment comprises cyclosporin A, Lumulse 40-L®, and Lemon oil.

Another embodiment comprises cyclosporin A, Lumulse 40-S®, and Lemon oil.

Another embodiment comprises cyclosporin A, Lumulse 42-L®, and Lemon oil.

Another embodiment comprises cyclosporin A, Lumulse 42-S®, and Lemon oil.

Another embodiment comprises cyclosporin A, Lumulse 100-S®, and Lemon oil.

Another embodiment comprises cyclosporin A, Lumulse 602-S®, and Lemon oil.

Another embodiment comprises cyclosporin A, Magrabar PGE-20-0®, and Lemon oil.

Another embodiment comprises cyclosporin A, Magrabar PGE-20L®, and Lemon oil.

Another embodiment comprises cyclosporin A, Magrabar PGE-20T®, and Lemon oil.

Another embodiment comprises cyclosporin A, Magrabar PGE-22-0®, and Lemon oil.

Another embodiment comprises cyclosporin A, Magrabar PGE-22L®, and Lemon oil.

Another embodiment comprises cyclosporin A, Magrabar PGE-22T®, and Lemon oil.

Another embodiment comprises cyclosporin A, Magrabar PGE-40-0®, and Lemon oil.

Another embodiment comprises cyclosporin A, Magrabar PGE-40L®, and Lemon oil.

Another embodiment comprises cyclosporin A, Magrabar PGE-40T®, and Lemon oil.

Another embodiment comprises cyclosporin A, Magrabar PGE-42-0®, and Lemon oil.

Another embodiment comprises cyclosporin A, Magrabar PGE-42L®, and Lemon oil.

Another embodiment comprises cyclosporin A, Magrabar PGE-42T®, and Lemon oil.

Another embodiment comprises cyclosporin A, Magrabar PGE-60-0®, and Lemon oil.

Another embodiment comprises cyclosporin A, Magrabar PGE-60L®, and Lemon oil.

Another embodiment comprises cyclosporin A, Magrabar PGE-60T®, and Lemon oil.

Another embodiment comprises cyclosporin A, Magrabar PGE-62-0®, and Lemon oil.

Another embodiment comprises cyclosporin A, Magrabar PGE-62L®, and Lemon oil.

Another embodiment comprises cyclosporin A, Magrabar PGE-62T®, and Lemon oil.

Another embodiment comprises cyclosporin A, Mapeg S-40K®, and Lemon oil.

Another embodiment comprises cyclosporin A, Marlowet OTS®, and Lemon oil.

Another embodiment comprises cyclosporin A, Naturechem PGR®, and Lemon oil.

Another embodiment comprises cyclosporin A, PG No. 4®, and Lemon oil.

Another embodiment comprises cyclosporin A, Renex 20®, and Lemon oil.

Another embodiment comprises cyclosporin A, Ritox 52®, and Lemon oil.

Another embodiment comprises cyclosporin A, Ritox 53®, and Lemon oil.

Another embodiment comprises cyclosporin A, Ritox 59®, and Lemon oil.

Another embodiment comprises cyclosporin A, Surfax 8916/A®, and Lemon oil.

Another embodiment comprises cyclosporin A, Tego Acid S 40 P®, and Lemon oil.

Another embodiment comprises cyclosporin A, Tego Acid S 100 P®, and Lemon oil.

Another embodiment comprises cyclosporin A, Tween 20®, and Lemon oil.

Another embodiment comprises cyclosporin A, Volpo 131®, and Lemon oil.

Another embodiment comprises cyclosporin A, an Ethoxylated Fatty Ester or Oil, and Lemon oil.

Another embodiment comprises cyclosporin A, Acconon 6-C10®, and Lemon oil.

Another embodiment comprises cyclosporin A, Acconon CC-6®, and Lemon oil.

Another embodiment comprises cyclosporin A, Acconon CO-7®, and Lemon oil.

Another embodiment comprises cyclosporin A, Aldosperse 40/60 FG®, and Lemon oil.

Another embodiment comprises cyclosporin A, Aldosperse ML-23®, and Lemon oil.

Another embodiment comprises cyclosporin A, Aldosperse MS-20 FG®, and Lemon oil.

Another embodiment comprises cyclosporin A, Alkamuls EL-620®, and Lemon oil.

Another embodiment comprises cyclosporin A, Alkamuls EL-719®, and Lemon oil.

Another embodiment comprises cyclosporin A, Alkamuls EL-985®, and Lemon oil.

Another embodiment comprises cyclosporin A, Arlatone G®, and Lemon oil.

Another embodiment comprises cyclosporin A, Arlatone T®, and Lemon oil.

Another embodiment comprises cyclosporin A, Atlas G-1045A®, and Lemon oil.

Another embodiment comprises cyclosporin A, Atlas G-1086®, and Lemon oil.

Another embodiment comprises cyclosporin A, Atlas G-1087®, and Lemon oil.

Another embodiment comprises cyclosporin A, Atlas G-1089®, and Lemon oil.

Another embodiment comprises cyclosporin A, Atlas G-1096®, and Lemon oil.

Another embodiment comprises cyclosporin A, Atlas G-1292®, and Lemon oil.

Another embodiment comprises cyclosporin A, Atlas G-1293®, and Lemon oil.

Another embodiment comprises cyclosporin A, Atlas G-1300®, and Lemon oil.

Another embodiment comprises cyclosporin A, Atlas G-7076®, and Lemon oil.

Another embodiment comprises cyclosporin A, Capmul EMG®, and Lemon oil.

Another embodiment comprises cyclosporin A, Chemonic CO-40®, and Lemon oil.

Another embodiment comprises cyclosporin A, Chemonic LI-3®, and Lemon oil.

Another embodiment comprises cyclosporin A, Chemonic LI-7®, and Lemon oil.

Another embodiment comprises cyclosporin A, Cirrasol GM®, and Lemon oil.

Another embodiment comprises cyclosporin A, Cremophor CO 40®, and Lemon oil.

Another embodiment comprises cyclosporin A, Cremophor CO 410®, and Lemon oil.

Another embodiment comprises cyclosporin A, Cremophor EL®, and Lemon oil.

Another embodiment comprises cyclosporin A, Cremophor GC7®, and Lemon oil.

Another embodiment comprises cyclosporin A, Cremophor RH-40®, and Lemon oil.

Another embodiment comprises cyclosporin A, Crovol A-40®, and Lemon oil.

Another embodiment comprises cyclosporin A, Crovol A-70®, and Lemon oil.

Another embodiment comprises cyclosporin A, Crovol M-70®, and Lemon oil.

Another embodiment comprises cyclosporin A, Crovol PK-70®, and Lemon oil.

Another embodiment comprises cyclosporin A, Cutina E-24®, and Lemon oil.

Another embodiment comprises cyclosporin A, Dacospin 12-R®, and Lemon oil.

Another embodiment comprises cyclosporin A, Dehymuls HRE-7®, and Lemon oil.

Another embodiment comprises cyclosporin A, DeSonic 30C®, and Lemon oil.

Another embodiment comprises cyclosporin A, DeSonic 36C®, and Lemon oil.

Another embodiment comprises cyclosporin A, DeSonic 40C®, and Lemon oil.

Another embodiment comprises cyclosporin A, Durfax 60®, and Lemon oil.

Another embodiment comprises cyclosporin A, Durfax 65®, and Lemon oil.

Another embodiment comprises cyclosporin A, Durfax 80®, and Lemon oil.

Another embodiment comprises cyclosporin A, Durfax EOM®, and Lemon oil.

Another embodiment comprises cyclosporin A, Eccoterge NF-2®, and Lemon oil.

Another embodiment comprises cyclosporin A, Emulpon CO-360®, and Lemon oil.

Another embodiment comprises cyclosporin A, Emulpon CO-550®, and Lemon oil.

Another embodiment comprises cyclosporin A, Emulsogen EL®, and Lemon oil.

Another embodiment comprises cyclosporin A, Emulsogen HCO 040®, and Lemon oil.

Another embodiment comprises cyclosporin A, Emulsogen HCO 060®, and Lemon oil.

Another embodiment comprises cyclosporin A, Emulsynt 1055®, and Lemon oil.

Another embodiment comprises cyclosporin A, Ethox 3095®, and Lemon oil.

Another embodiment comprises cyclosporin A, Eumulgin RO-40®, and Lemon oil.

Another embodiment comprises cyclosporin A, Genapol G-260®, and Lemon oil.

Another embodiment comprises cyclosporin A, Glycosperse L-20®, and Lemon oil.

Another embodiment comprises cyclosporin A, Glycosperse O-5®, and Lemon oil.

Another embodiment comprises cyclosporin A, Glycosperse O-20®, and Lemon oil.

Another embodiment comprises cyclosporin A, Glycosperse O-20 FG®, and Lemon oil.

Another embodiment comprises cyclosporin A, Glycosperse S-20®, and Lemon oil.

Another embodiment comprises cyclosporin A, Glycosperse S-20 FG®, and Lemon oil.

Another embodiment comprises cyclosporin A, Glycosperse TS-20®, and Lemon oil.

Another embodiment comprises cyclosporin A, Glycosperse TS-20 FG®, and Lemon oil.

Another embodiment comprises cyclosporin A, Hetan SL®, and Lemon oil.

Another embodiment comprises cyclosporin A, Hetan SO®, and Lemon oil.

Another embodiment comprises cyclosporin A, Hetan SS®, and Lemon oil.

Another embodiment comprises cyclosporin A, Hetoxide C-2®, and Lemon oil.

Another embodiment comprises cyclosporin A, Hetoxide C-9®, and Lemon oil.

Another embodiment comprises cyclosporin A, Hetoxide C-15®, and Lemon oil.

Another embodiment comprises cyclosporin A, Hetoxide C-25®, and Lemon oil.

Another embodiment comprises cyclosporin A, Hetoxide C-40®, and Lemon oil.

Another embodiment comprises cyclosporin A, Hetoxide C-200®, and Lemon oil.

Another embodiment comprises cyclosporin A, Hetoxide C-200-50%®, and Lemon oil.

Another embodiment comprises cyclosporin A, Hetoxide GC-30®, and Lemon oil.

Another embodiment comprises cyclosporin A, Hetoxide HC-60®, and Lemon oil.

Another embodiment comprises cyclosporin A, Ice No. 2®, and Lemon oil.

Another embodiment comprises cyclosporin A, Incrocas 30/40®, and Lemon oil.

Another embodiment comprises cyclosporin A, Lexol EC®, and Lemon oil.

Another embodiment comprises cyclosporin A, Lexol EO®, and Lemon oil.

Another embodiment comprises cyclosporin A, Lipocol HCO-40®, and Lemon oil.

Another embodiment comprises cyclosporin A, Lipocol HCO-60®, and Lemon oil.

Another embodiment comprises cyclosporin A, Lipocol O-3 Special®, and Lemon oil.

Another embodiment comprises cyclosporin A, Lipopeg 2-L®, and Lemon oil.

Another embodiment comprises cyclosporin A, Lipopeg 4-DO®, and Lemon oil.

Another embodiment comprises cyclosporin A, Lipopeg 4-DS®, and Lemon oil.

Another embodiment comprises cyclosporin A, Lipovol GTB®, and Lemon oil.

Another embodiment comprises cyclosporin A, Lonzest SML-20®, and Lemon oil.

Another embodiment comprises cyclosporin A, Lonzest SMO-20®, and Lemon oil.

Another embodiment comprises cyclosporin A, Lonzest SMS-20®, and Lemon oil.

Another embodiment comprises cyclosporin A, Lonzest STO-20®, and Lemon oil.

Another embodiment comprises cyclosporin A, Lonzest STS-20®, and Lemon oil.

Another embodiment comprises cyclosporin A, Lumulse GR-40®, and Lemon oil.

Another embodiment comprises cyclosporin A, Lumulse GRH-40®, and Lemon oil.

Another embodiment comprises cyclosporin A, Lumulse POE (7) GML®, and Lemon oil.

Another embodiment comprises cyclosporin A, Lumulse POE (12) Glyc®, and Lemon oil.

Another embodiment comprises cyclosporin A, Lumulse POE (40) MS KP®, and Lemon oil.

Another embodiment comprises cyclosporin A, Marlowet 4750®, and Lemon oil.

Another embodiment comprises cyclosporin A, Marlowet LVS®, and Lemon oil.

Another embodiment comprises cyclosporin A, Marlowet R 11®, and Lemon oil.

Another embodiment comprises cyclosporin A, Marlowet R 40®, and Lemon oil.

Another embodiment comprises cyclosporin A, Mazol 80 MGK®, and Lemon oil.

Another embodiment comprises cyclosporin A, Nonionic Emulsifier T-9®, and Lemon oil.

Another embodiment comprises cyclosporin A, Oronal LCG®, and Lemon oil.

Another embodiment comprises cyclosporin A, Polyderm PPI-CO-200®, and Lemon oil.

Another embodiment comprises cyclosporin A, Polyderm PPI-CO-40®, and Lemon oil.

Another embodiment comprises cyclosporin A, Rewoderm LI 520-70®, and Lemon oil.

Another embodiment comprises cyclosporin A, Ritapeg 150 DS®, and Lemon oil.

Another embodiment comprises cyclosporin A, Softigen 767®, and Lemon oil.

Another embodiment comprises cyclosporin A, Surfactol 318®, and Lemon oil.

Another embodiment comprises cyclosporin A, Surfactol 365®, and Lemon oil.

Another embodiment comprises cyclosporin A, Syn Lube 107®, and Lemon oil.

Another embodiment comprises cyclosporin A, Syn Lube 728®, and Lemon oil.

Another embodiment comprises cyclosporin A, Syn Lube 1632H®, and Lemon oil.

Another embodiment comprises cyclosporin A, Syn Lube 6277-A®, and Lemon oil.

Another embodiment comprises cyclosporin A, T-Det C-20®, and Lemon oil.

Another embodiment comprises cyclosporin A, T-Det C-40®, and Lemon oil.

Another embodiment comprises cyclosporin A, Tally 100 Plus®, and Lemon oil.

Another embodiment comprises cyclosporin A, Uniperol EL®, and Lemon oil.

Another embodiment comprises cyclosporin A, a Fatty Ester, and Lemon oil.

Another embodiment comprises cyclosporin A, Actralube-Syn 147®, and Lemon oil.

Another embodiment comprises cyclosporin A, Atlas G-1556®, and Lemon oil.

Another embodiment comprises cyclosporin A, Atlas G-1564®, and Lemon oil.

Another embodiment comprises cyclosporin A, Atlasol Base Oil S®, and Lemon oil.

Another embodiment comprises cyclosporin A, Base ML®, and Lemon oil.

Another embodiment comprises cyclosporin A, Base MT®, and Lemon oil.

Another embodiment comprises cyclosporin A, Cerasynt 303®, and Lemon oil.

Another embodiment comprises cyclosporin A, Dermol 1012®, and Lemon oil.

Another embodiment comprises cyclosporin A, Kemester 4000®, and Lemon oil.

Another embodiment comprises cyclosporin A, Lactipol S®, and Lemon oil.

Another embodiment comprises cyclosporin A, Magrabar PGO®, and Lemon oil.

Another embodiment comprises cyclosporin A, Mayco Base BFO®, and Lemon oil.

Another embodiment comprises cyclosporin A, Methyl Linoleate®, and Lemon oil.

Another embodiment comprises cyclosporin A, Pationic 122A®, and Lemon oil.

Another embodiment comprises cyclosporin A, Pationic 138C®, and Lemon oil.

Another embodiment comprises cyclosporin A, Pationic CSL®, and Lemon oil.

Another embodiment comprises cyclosporin A, Pationic ISL®, and Lemon oil.

Another embodiment comprises cyclosporin A, Pationic SBL®, and Lemon oil.

Another embodiment comprises cyclosporin A, Pationic SSL®, and Lemon oil.

Another embodiment comprises cyclosporin A, Ritasol®, and Lemon oil.

Another embodiment comprises cyclosporin A, Tego Alkanol CS 20®, and Lemon oil.

Another embodiment comprises cyclosporin A, Tego Alkanol L23 P®, and Lemon oil.

Another embodiment comprises cyclosporin A, Tego Alkanol S2®, and Lemon oil.

Another embodiment comprises cyclosporin A, Tego Alkanol S20 P®, and Lemon oil.

Another embodiment comprises cyclosporin A, Triemulsifier 600 MS®, and Lemon oil.

Another embodiment comprises cyclosporin A, a Glycerol Esters, and Lemon oil.

Another embodiment comprises cyclosporin A, Agro #9 Wint SBO®, and Lemon oil.

Another embodiment comprises cyclosporin A, Ahcovel Base 700®, and Lemon oil.

Another embodiment comprises cyclosporin A, Aldo HMS FG®, and Lemon oil.

Another embodiment comprises cyclosporin A, Aldo MLD®, and Lemon oil.

Another embodiment comprises cyclosporin A, Aldo MLD FG®, and Lemon oil.

Another embodiment comprises cyclosporin A, Aldo MO FG®, and Lemon oil.

Another embodiment comprises cyclosporin A, Aldo MS®, and Lemon oil.

Another embodiment comprises cyclosporin A, Aldo MS FG®, and Lemon oil.

Another embodiment comprises cyclosporin A, Aldo MS LG FG®, and Lemon oil.

Another embodiment comprises cyclosporin A, Aldo MSD®, and Lemon oil.

Another embodiment comprises cyclosporin A, Aldo MSD FG®, and Lemon oil.

Another embodiment comprises cyclosporin A, Aldosperse O-20 FG®, and Lemon oil.

Another embodiment comprises cyclosporin A, Aldosperse TS-20 FG®, and Lemon oil.

Another embodiment comprises cyclosporin A, Aldosperse TS-40 FG®, and Lemon oil.

Another embodiment comprises cyclosporin A, Arlacel 165®, and Lemon oil.

Another embodiment comprises cyclosporin A, Arlacel 186®, and Lemon oil.

Another embodiment comprises cyclosporin A, Capmul GMO®, and Lemon oil.

Another embodiment comprises cyclosporin A, Capmul GMS®, and Lemon oil.

Another embodiment comprises cyclosporin A, Caprol 3GO®, and Lemon oil.

Another embodiment comprises cyclosporin A, Caprol 3GVS®, and Lemon oil.

Another embodiment comprises cyclosporin A, Caprol 6G2S®, and Lemon oil.

Another embodiment comprises cyclosporin A, Caprol 10G40®, and Lemon oil.

Another embodiment comprises cyclosporin A, Caprol 10G100®, and Lemon oil.

Another embodiment comprises cyclosporin A, Caprol ET®, and Lemon oil.

Another embodiment comprises cyclosporin A, Caprol PGE860®, and Lemon oil.

Another embodiment comprises cyclosporin A, Cerasynt 945®, and Lemon oil.

Another embodiment comprises cyclosporin A, Cerasynt GMS®, and Lemon oil.

Another embodiment comprises cyclosporin A, Cerasynt Q®, and Lemon oil.

Another embodiment comprises cyclosporin A, Cerasynt SD®, and Lemon oil.

Another embodiment comprises cyclosporin A, Cerasynt WM®, and Lemon oil.

Another embodiment comprises cyclosporin A, Chemsperse 14®, and Lemon oil.

Another embodiment comprises cyclosporin A, Cremophor GO-32®, and Lemon oil.

Another embodiment comprises cyclosporin A, Cremophor GS11®, and Lemon oil.

Another embodiment comprises cyclosporin A, Cremophor GS-32®, and Lemon oil.

Another embodiment comprises cyclosporin A, Cutina KD-16®, and Lemon oil.

Another embodiment comprises cyclosporin A, Dehymuls PGPH®, and Lemon oil.

Another embodiment comprises cyclosporin A, Dermol DGDIS®, and Lemon oil.

Another embodiment comprises cyclosporin A, Dermol DGMIS®, and Lemon oil.

Another embodiment comprises cyclosporin A, Dermol G-76®, and Lemon oil.

Another embodiment comprises cyclosporin A, Dermol G-7DI®, and Lemon oil.

Another embodiment comprises cyclosporin A, Dermol NGDI®, and Lemon oil.

Another embodiment comprises cyclosporin A, Dermolan GLH®, and Lemon oil.

Another embodiment comprises cyclosporin A, Drewmulse GMO®, and Lemon oil.

Another embodiment comprises cyclosporin A, Drewpol 3-5-M®, and Lemon oil.

Another embodiment comprises cyclosporin A, Durlac 100 W®, and Lemon oil.

Another embodiment comprises cyclosporin A, Dur-Lo®, and Lemon oil.

Another embodiment comprises cyclosporin A, Dynasan 118®, and Lemon oil.

Another embodiment comprises cyclosporin A, EC-25®, and Lemon oil.

Another embodiment comprises cyclosporin A, EM 40®, and Lemon oil.

Another embodiment comprises cyclosporin A, Emerest 2400®, and Lemon oil.

Another embodiment comprises cyclosporin A, Emerest 2452®, and Lemon oil.

Another embodiment comprises cyclosporin A, Empilan G-26®, and Lemon oil.

Another embodiment comprises cyclosporin A, Genapol TSM®, and Lemon oil.

Another embodiment comprises cyclosporin A, Hostacerin DGI®, and Lemon oil.

Another embodiment comprises cyclosporin A, Hostacerin DGL®, and Lemon oil.

Another embodiment comprises cyclosporin A, Hostacerin DGMS®, and Lemon oil.

Another embodiment comprises cyclosporin A, Hostacerin DGSB®, and Lemon oil.

Another embodiment comprises cyclosporin A, Ice No. 2®, and Lemon oil.

Another embodiment comprises cyclosporin A, Imwitor 742®, and Lemon oil.

Another embodiment comprises cyclosporin A, Imwitor 780 K®, and Lemon oil.

Another embodiment comprises cyclosporin A, Imwitor 960 Flakes®, and Lemon oil.

Another embodiment comprises cyclosporin A, isolan GI 34®, and Lemon oil.

Another embodiment comprises cyclosporin A, Isolan GO 33®, and Lemon oil.

Another embodiment comprises cyclosporin A, Kemester 1000®, and Lemon oil.

Another embodiment comprises cyclosporin A, Kemester 2000®, and Lemon oil.

Another embodiment comprises cyclosporin A, Kemester 2000®, and Lemon oil.

Another embodiment comprises cyclosporin A, Kemester 6000SE®, and Lemon oil.

Another embodiment comprises cyclosporin A, Lamecreme DGE 18®, and Lemon oil.

Another embodiment comprises cyclosporin A, Lexemul 515®, and Lemon oil.

Another embodiment comprises cyclosporin A, Lexemul 561®, and Lemon oil.

Another embodiment comprises cyclosporin A, Lexemul AR®, and Lemon oil.

Another embodiment comprises cyclosporin A, Lexemul AS®, and Lemon oil.

Another embodiment comprises cyclosporin A, Lexemul GDL®, and Lemon oil.

Another embodiment comprises cyclosporin A, Lexemul T®, and Lemon oil.

Another embodiment comprises cyclosporin A, Lipomulse 165®, and Lemon oil.

Another embodiment comprises cyclosporin A, Lumulse GML K®, and Lemon oil.

Another embodiment comprises cyclosporin A, Lumulse GMO K®, and Lemon oil.

Another embodiment comprises cyclosporin A, Lumulse GMR K®, and Lemon oil.

Another embodiment comprises cyclosporin A, Lumulse GMT K®, and Lemon oil.

Another embodiment comprises cyclosporin A, Magrabar GMC®, and Lemon oil.

Another embodiment comprises cyclosporin A, Magrabar GMO-CK®, and Lemon oil.

Another embodiment comprises cyclosporin A, Magrabar MDG-5050®, and Lemon oil.

Another embodiment comprises cyclosporin A, Magrabar PGO-315®, and Lemon oil.

Another embodiment comprises cyclosporin A, Magrabar PGO-1010®, and Lemon oil.

Another embodiment comprises cyclosporin A, Mazol 300K®, and Lemon oil.

Another embodiment comprises cyclosporin A, Mazol GMO-K®, and Lemon oil.

Another embodiment comprises cyclosporin A, Mazol GMS-K®, and Lemon oil.

Another embodiment comprises cyclosporin A, Mazol PG031-K®, and Lemon oil.

Another embodiment comprises cyclosporin A, Miglyol 812®, and Lemon oil.

Another embodiment comprises cyclosporin A, Norfox 165C®, and Lemon oil.

Another embodiment comprises cyclosporin A, Schercemol GMIS®, and Lemon oil.

Another embodiment comprises cyclosporin A, Tegin®, and Lemon oil.

Another embodiment comprises cyclosporin A, Tegin 4100 Pellets®, and Lemon oil.

Another embodiment comprises cyclosporin A, Tegin M Pellets®, and Lemon oil.

Another embodiment comprises cyclosporin A, Tegin OV®, and Lemon oil.

Another embodiment comprises cyclosporin A, Teginacid H®, and Lemon oil.

Another embodiment comprises cyclosporin A, Tego Cosmo P813®, and Lemon oil.

Another embodiment comprises cyclosporin A, Wickenol 535®, and Lemon oil.

Another embodiment comprises cyclosporin A, Witconol 14®, and Lemon oil.

Another embodiment comprises cyclosporin A, Witconol 14®, and Lemon oil.

Another embodiment comprises cyclosporin A, Witconol 14F®, and Lemon oil.

Another embodiment comprises cyclosporin A, Witconol 18L®, and Lemon oil.

Another embodiment comprises cyclosporin A, Witconol GOT®, and Lemon oil.

Another embodiment comprises cyclosporin A, Witconol MST®, and Lemon oil.

Another embodiment comprises cyclosporin A, Witconol RHT®, and Lemon oil.

Another embodiment comprises cyclosporin A, a Glycol Ester, and Lemon oil.

Another embodiment comprises cyclosporin A, Alkamuls 600 DO®, and Lemon oil.

Another embodiment comprises cyclosporin A, Alkamuls SEG®, and Lemon oil.

Another embodiment comprises cyclosporin A, Atlas EM-2®, and Lemon oil.

Another embodiment comprises cyclosporin A, Cerasynt IP®, and Lemon oil.

Another embodiment comprises cyclosporin A, Cerasynt M®, and Lemon oil.

Another embodiment comprises cyclosporin A, Cerasynt MN®, and Lemon oil.

Another embodiment comprises cyclosporin A, Cerasynt PA®, and Lemon oil.

Another embodiment comprises cyclosporin A, Chemsperse EGDS®, and Lemon oil.

Another embodiment comprises cyclosporin A, Chemsperse EGMS®, and Lemon oil.

Another embodiment comprises cyclosporin A, Colonial Monolaurin®, and Lemon oil.

Another embodiment comprises cyclosporin A, DeMuls SGE-95®, and Lemon oil.

Another embodiment comprises cyclosporin A, Eccoterge 200®, and Lemon oil.

Another embodiment comprises cyclosporin A, Emerest 2380®, and Lemon oil.

Another embodiment comprises cyclosporin A, Ethox 2610®, and Lemon oil.

Another embodiment comprises cyclosporin A, Ethox DO-9®, and Lemon oil.

Another embodiment comprises cyclosporin A, Ethox DO-14®, and Lemon oil.

Another embodiment comprises cyclosporin A, Ethox SO-9®, and Lemon oil.

Another embodiment comprises cyclosporin A, Fizul MD-318®, and Lemon oil.

Another embodiment comprises cyclosporin A, Genapol EGDS-VHP®, and Lemon oil.

Another embodiment comprises cyclosporin A, Genapol TS Powder®, and Lemon oil.

Another embodiment comprises cyclosporin A, Hostacerin WO®, and Lemon oil.

Another embodiment comprises cyclosporin A, Inversol 140®, and Lemon oil.

Another embodiment comprises cyclosporin A, Kemester 104®, and Lemon oil.

Another embodiment comprises cyclosporin A, Kemester 205®, and Lemon oil.

Another embodiment comprises cyclosporin A, Kemester 226®, and Lemon oil.

Another embodiment comprises cyclosporin A, Kemester 5221SE®, and Lemon oil.

Another embodiment comprises cyclosporin A, Kemester EGDS®, and Lemon oil.

Another embodiment comprises cyclosporin A, Lexemul EGDS®, and Lemon oil.

Another embodiment comprises cyclosporin A, Lexemul EGMS®, and Lemon oil.

Another embodiment comprises cyclosporin A, Lexemul P®, and Lemon oil.

Another embodiment comprises cyclosporin A, Lipo DGLS®, and Lemon oil.

Another embodiment comprises cyclosporin A, Lipo EGDS®, and Lemon oil.

Another embodiment comprises cyclosporin A, Lipo PGMS®, and Lemon oil.

Another embodiment comprises cyclosporin A, Liposorb S-4®, and Lemon oil.

Another embodiment comprises cyclosporin A, Liposorb TO-20®, and Lemon oil.

Another embodiment comprises cyclosporin A, Lumulse PGO®, and Lemon oil.

Another embodiment comprises cyclosporin A, Mackester EGDS®, and Lemon oil.

Another embodiment comprises cyclosporin A, Mackester EGMS®, and Lemon oil.

Another embodiment comprises cyclosporin A, Mackester GSTP®, and Lemon oil.

Another embodiment comprises cyclosporin A, Mackester Series®, and Lemon oil.

Another embodiment comprises cyclosporin A, Magrabar PDG-50®, and Lemon oil.

Another embodiment comprises cyclosporin A, Mapeg 6000 DS®, and Lemon oil.

Another embodiment comprises cyclosporin A, Marlowet 4702®, and Lemon oil.

Another embodiment comprises cyclosporin A, Monalube 305®, and Lemon oil.

Another embodiment comprises cyclosporin A, Monalube 310®, and Lemon oil.

Another embodiment comprises cyclosporin A, Monalube 315®, and Lemon oil.

Another embodiment comprises cyclosporin A, Monalube 320®, and Lemon oil.

Another embodiment comprises cyclosporin A, Monalube 325®, and Lemon oil.

Another embodiment comprises cyclosporin A, Monalube 330®, and Lemon oil.

Another embodiment comprises cyclosporin A, Naturechem PGHS®, and Lemon oil.

Another embodiment comprises cyclosporin A, Polycastorol PLO-840®, and Lemon oil.

Another embodiment comprises cyclosporin A, Polytex 10M®, and Lemon oil.

Another embodiment comprises cyclosporin A, Ritasynt IP®, and Lemon oil.

Another embodiment comprises cyclosporin A, Ross Chem PEG 600 DT®, and Lemon oil.

Another embodiment comprises cyclosporin A, Schercemol PGMS®, and Lemon oil.

Another embodiment comprises cyclosporin A, Sponto H-44C®, and Lemon oil.

Another embodiment comprises cyclosporin A, Tegin G®, and Lemon oil.

Another embodiment comprises cyclosporin A, Witbreak DGE-182®, and Lemon oil.

Another embodiment comprises cyclosporin A, Witbreak DGE-182®, and Lemon oil.

Another embodiment comprises cyclosporin A, Witbreak DRA-21®, and Lemon oil.

Another embodiment comprises cyclosporin A, Witbreak DRA-21®, and Lemon oil.

Another embodiment comprises cyclosporin A, Witbreak DRA-50®, and Lemon oil.

Another embodiment comprises cyclosporin A, Witbreak DRA-50®, and Lemon oil.

Another embodiment comprises cyclosporin A, Witconol F26-46®, and Lemon oil.

Another embodiment comprises cyclosporin A, Witconol H-32®, and Lemon oil.

Another embodiment comprises cyclosporin A, Witconol H-33®, and Lemon oil.

Another embodiment comprises cyclosporin A, Witconol H-35A®, and Lemon oil.

Another embodiment comprises cyclosporin A, Witconol RHP®, and Lemon oil.

Another embodiment comprises cyclosporin A, a Lanolin-based Derivative, and Lemon oil.

Another embodiment comprises cyclosporin A, Amerchol CAB®, and Lemon oil.

Another embodiment comprises cyclosporin A, Amerchol L-101®, and Lemon oil.

Another embodiment comprises cyclosporin A, Amerlate LFA-LO®, and Lemon oil.

Another embodiment comprises cyclosporin A, Amerlate P®, and Lemon oil.

Another embodiment comprises cyclosporin A, Barre Common Degras®, and Lemon oil.

Another embodiment comprises cyclosporin A, Cholesterol NF®, and Lemon oil.

Another embodiment comprises cyclosporin A, Crodalan AWS®, and Lemon oil.

Another embodiment comprises cyclosporin A, Crodalan LA®, and Lemon oil.

Another embodiment comprises cyclosporin A, Emery 1650®, and Lemon oil.

Another embodiment comprises cyclosporin A, Emery 1650®, and Lemon oil.

Another embodiment comprises cyclosporin A, Emery 1740®, and Lemon oil.

Another embodiment comprises cyclosporin A, Emery 1740®, and Lemon oil.

Another embodiment comprises cyclosporin A, Forlan 500®, and Lemon oil.

Another embodiment comprises cyclosporin A, Forlan L®, and Lemon oil.

Another embodiment comprises cyclosporin A, Laneto 50®, and Lemon oil.

Another embodiment comprises cyclosporin A, Laneto 100®, and Lemon oil.

Another embodiment comprises cyclosporin A, Laneto AWS®, and Lemon oil.

Another embodiment comprises cyclosporin A, Lanfrax 1776®, and Lemon oil.

Another embodiment comprises cyclosporin A, Lanfrax 1776®, and Lemon oil.

Another embodiment comprises cyclosporin A, Lanogel 21®, and Lemon oil.

Another embodiment comprises cyclosporin A, Lipolan®, and Lemon oil.

Another embodiment comprises cyclosporin A, Lipolan 31®, and Lemon oil.

Another embodiment comprises cyclosporin A, OHlan®, and Lemon oil.

Another embodiment comprises cyclosporin A, Polychol 5®, and Lemon oil.

Another embodiment comprises cyclosporin A, Polychol 15®, and Lemon oil.

Another embodiment comprises cyclosporin A, Ritacetyl®, and Lemon oil.

Another embodiment comprises cyclosporin A, Ritachol®, and Lemon oil.

Another embodiment comprises cyclosporin A, Ritahydrox®, and Lemon oil.

Another embodiment comprises cyclosporin A, Ritalafa®, and Lemon oil.

Another embodiment comprises cyclosporin A, Ritalan®, and Lemon oil.

Another embodiment comprises cyclosporin A, Ritalan AWS®, and Lemon oil.

Another embodiment comprises cyclosporin A, Ritalan C®, and Lemon oil.

Another embodiment comprises cyclosporin A, Ritawax®, and Lemon oil.

Another embodiment comprises cyclosporin A, Ritawax AEO®, and Lemon oil.

Another embodiment comprises cyclosporin A, Ritawax ALA®, and Lemon oil.

Another embodiment comprises cyclosporin A, Solan/Solan 50/Super Solan®, and Lemon oil.

Another embodiment comprises cyclosporin A, Super Hartolan/Hartolan®, and Lemon oil.

Another embodiment comprises cyclosporin A, Supersat AWS-4®, and Lemon oil.

Another embodiment comprises cyclosporin A, Supersat AWS-24®, and Lemon oil.

Another embodiment comprises cyclosporin A, a Lecithin or Lecithin Derivative, and Lemon oil.

Another embodiment comprises cyclosporin A, Alcolec®, and Lemon oil.

Another embodiment comprises cyclosporin A, Lecithin®, and Lemon oil.

Another embodiment comprises cyclosporin A, Lexin K®, and Lemon oil.

Another embodiment comprises cyclosporin A, Natipide®, and Lemon oil.

Another embodiment comprises cyclosporin A, a Lignin or Lignin Derivative, and Lemon oil.

Another embodiment comprises cyclosporin A, Diwatex XP 9®, and Lemon oil.

Another embodiment comprises cyclosporin A, Dynasperse LCD®, and Lemon oil.

Another embodiment comprises cyclosporin A, Indulin SAL®, and Lemon oil.

Another embodiment comprises cyclosporin A, Indulin W-1®, and Lemon oil.

Another embodiment comprises cyclosporin A, Indulin W-5®, and Lemon oil.

Another embodiment comprises cyclosporin A, Lignosol FTA®, and Lemon oil.

Another embodiment comprises cyclosporin A, Lignosol SFX-65®, and Lemon oil.

Another embodiment comprises cyclosporin A, Marasperse 52 CP®, and Lemon oil.

Another embodiment comprises cyclosporin A, Marasperse AG®, and Lemon oil.

Another embodiment comprises cyclosporin A, Marasperse CBOS-48, and Lemon oil.

Another embodiment comprises cyclosporin A, Ufoxane 2®, and Lemon oil.

Another embodiment comprises cyclosporin A, a Methyl Ester, and Lemon oil.

Another embodiment comprises cyclosporin A, E.B. Cleaner AK®, and Lemon oil.

Another embodiment comprises cyclosporin A, Oleocal ME-70®, and Lemon oil.

Another embodiment comprises cyclosporin A, Trade Name Company®, and Lemon oil.

Another embodiment comprises cyclosporin A, Methyl Esters (cont'd)®, and Lemon oil.

Another embodiment comprises cyclosporin A, Oleocal ME-92®, and Lemon oil.

Another embodiment comprises cyclosporin A, Oleocal ME-112®, and Lemon oil.

Another embodiment comprises cyclosporin A, Oleocal ME-130®, and Lemon oil.

Another embodiment comprises cyclosporin A, a Monoglyceride or a Derivative thereof, and Lemon oil.

Another embodiment comprises cyclosporin A, Dynacet 211®, and Lemon oil.

Another embodiment comprises cyclosporin A, Hetsorb S-20®, and Lemon oil.

Another embodiment comprises cyclosporin A, Imwitor 191®, and Lemon oil.

Another embodiment comprises cyclosporin A, Imwitor 370®, and Lemon oil.

Another embodiment comprises cyclosporin A, Imwitor 375®, and Lemon oil.

Another embodiment comprises cyclosporin A, Imwitor 900®, and Lemon oil.

Another embodiment comprises cyclosporin A, Imwitor 945®, and Lemon oil.

Another embodiment comprises cyclosporin A, Imwitor 2020®, and Lemon oil.

Another embodiment comprises cyclosporin A, Kemester 5500®, and Lemon oil.

Another embodiment comprises cyclosporin A, Kemester 6000®, and Lemon oil.

Another embodiment comprises cyclosporin A, Magrabar GMC®, and Lemon oil.

Another embodiment comprises cyclosporin A, Magrabar GMO-CK®, and Lemon oil.

Another embodiment comprises cyclosporin A, Magrabar GPC-10®, and Lemon oil.

Another embodiment comprises cyclosporin A, Magrabar MDG-5050®, and Lemon oil.

Another embodiment comprises cyclosporin A, Monalube 335®, and Lemon oil.

Another embodiment comprises cyclosporin A, Monoglycerides Glyceryl Monestearate Archer Daniels Midland Company®, and Lemon oil.

Another embodiment comprises cyclosporin A, Rita GMS®, and Lemon oil.

Another embodiment comprises cyclosporin A, Ritamulse SCG®, and Lemon oil.

Another embodiment comprises cyclosporin A, Softigen 701®, and Lemon oil.

Another embodiment comprises cyclosporin A, Tally 100 Plus®, and Lemon oil.

Another embodiment comprises cyclosporin A, a Polyethylene Glycol, and Lemon oil.

Another embodiment comprises cyclosporin A, Emulgade PL 68/50®, and Lemon oil.

Another embodiment comprises cyclosporin A, Lumulse PEG®, and Lemon oil.

Another embodiment comprises cyclosporin A, Rhodasurf PEG-400®, and Lemon oil.

Another embodiment comprises cyclosporin A, Rhodasurf PEG-600®, and Lemon oil.

Another embodiment comprises cyclosporin A, Witconol PEG-400®, and Lemon oil.

Another embodiment comprises cyclosporin A, a Polymeric Sudactant, and Lemon oil.

Another embodiment comprises cyclosporin A, Acritamer PNC-EG®, and Lemon oil.

Another embodiment comprises cyclosporin A, Ag-Rho DEP-775®, and Lemon oil.

Another embodiment comprises cyclosporin A, APG 325N Glycoside®, and Lemon oil.

Another embodiment comprises cyclosporin A, Aristoflex AVC®, and Lemon oil.

Another embodiment comprises cyclosporin A, Aristoflex HMB®, and Lemon oil.

Another embodiment comprises cyclosporin A, Burco NPS-225®, and Lemon oil.

Another embodiment comprises cyclosporin A, Burco NPS-816®, and Lemon oil.

Another embodiment comprises cyclosporin A, Chemccinate 5603®, and Lemon oil.

Another embodiment comprises cyclosporin A, Cosmedia Guar C-261 N®, and Lemon oil.

Another embodiment comprises cyclosporin A, Gantrez S-95®, and Lemon oil.

Another embodiment comprises cyclosporin A, Glucopon 220 UP®, and Lemon oil.

Another embodiment comprises cyclosporin A, Glucopon 225 DK®, and Lemon oil.

Another embodiment comprises cyclosporin A, Glucopon 425 N®, and Lemon oil.

Another embodiment comprises cyclosporin A, Glucopon 600 UP®, and Lemon oil.

Another embodiment comprises cyclosporin A, Glucopon 625 UP®, and Lemon oil.

Another embodiment comprises cyclosporin A, Pemulen 1621®, and Lemon oil.

Another embodiment comprises cyclosporin A, Pemulen 1622®, and Lemon oil.

Another embodiment comprises cyclosporin A, Pemulen TR-1®, and Lemon oil.

Another embodiment comprises cyclosporin A, Pemulen TR-2®, and Lemon oil.

Another embodiment comprises cyclosporin A, Plantacare 818®, and Lemon oil.

Another embodiment comprises cyclosporin A, Plantapon LGC Sorb®, and Lemon oil.

Another embodiment comprises cyclosporin A, Plantaren 1200N®, and Lemon oil.

Another embodiment comprises cyclosporin A, Plantaren 2000N®, and Lemon oil.

Another embodiment comprises cyclosporin A, Viscolam AT 64®, and Lemon oil.

Another embodiment comprises cyclosporin A, Viscolam AT 64P®, and Lemon oil.

Another embodiment comprises cyclosporin A, Viscolam AT 100®, and Lemon oil.

Another embodiment comprises cyclosporin A, Viscolam MAC 7®, and Lemon oil.

Another embodiment comprises cyclosporin A, Viscolam SMC 20®, and Lemon oil.

Another embodiment comprises cyclosporin A, Witbreak RTC-323®, and Lemon oil.

Another embodiment comprises cyclosporin A, WSI 3700®, and Lemon oil.

Another embodiment comprises cyclosporin A, a Propoxylated or Ethoxylated Fatty Acid, Alcohol, or Alkyl Phenol, and Lemon oil.

Another embodiment comprises cyclosporin A, Antarox AA-60®, and Lemon oil.

Another embodiment comprises cyclosporin A, Antarox LF-224®, and Lemon oil.

Another embodiment comprises cyclosporin A, Burcomul DFE-45®, and Lemon oil.

Another embodiment comprises cyclosporin A, Burcoterge LFE-1000®, and Lemon oil.

Another embodiment comprises cyclosporin A, Chemal LF-25B®, and Lemon oil.

Another embodiment comprises cyclosporin A, Chemal LF-40B®, and Lemon oil.

Another embodiment comprises cyclosporin A, Dehypon LS-36®, and Lemon oil.

Another embodiment comprises cyclosporin A, Dehypon LS-36®, and Lemon oil.

Another embodiment comprises cyclosporin A, Dehypon LS-54®, and Lemon oil.

Another embodiment comprises cyclosporin A, Dehypon LS-54®, and Lemon oil.

Another embodiment comprises cyclosporin A, Delonic 100 VLF®, and Lemon oil.

Another embodiment comprises cyclosporin A, Delonic LF-60 MOD®, and Lemon oil.

Another embodiment comprises cyclosporin A, Empiderm B®, and Lemon oil.

Another embodiment comprises cyclosporin A, Ethylan 1206®, and Lemon oil.

Another embodiment comprises cyclosporin A, Ethylan NS-500K®, and Lemon oil.

Another embodiment comprises cyclosporin A, Ethylan NS-500LQ®, and Lemon oil.

Another embodiment comprises cyclosporin A, Genapol 1392®, and Lemon oil.

Another embodiment comprises cyclosporin A, Genapol 2317®, and Lemon oil.

Another embodiment comprises cyclosporin A, Genapol 26EP710®, and Lemon oil.

Another embodiment comprises cyclosporin A, Genapol EP 1022®, and Lemon oil.

Another embodiment comprises cyclosporin A, Genapol EP 1024®, and Lemon oil.

Another embodiment comprises cyclosporin A, Genapol EP 6068®, and Lemon oil.

Another embodiment comprises cyclosporin A, Genapol NP915®, and Lemon oil.

Another embodiment comprises cyclosporin A, Kieralon MFB®, and Lemon oil.

Another embodiment comprises cyclosporin A, Lumisolve CSA-80 V®, and Lemon oil.

Another embodiment comprises cyclosporin A, Marlowet 5001®, and Lemon oil.

Another embodiment comprises cyclosporin A, Marlox FK 64®, and Lemon oil.

Another embodiment comprises cyclosporin A, Marlox MO 124®, and Lemon oil.

Another embodiment comprises cyclosporin A, Marlox S 58®, and Lemon oil.

Another embodiment comprises cyclosporin A, Nonatell 1003®, and Lemon oil.

Another embodiment comprises cyclosporin A, Nonatell 1038®, and Lemon oil.

Another embodiment comprises cyclosporin A, Nonatell 1052®, and Lemon oil.

Another embodiment comprises cyclosporin A, Nonatell 1061®, and Lemon oil.

Another embodiment comprises cyclosporin A, Nonatell 1075®, and Lemon oil.

Another embodiment comprises cyclosporin A, Nonatell 1088®, and Lemon oil.

Another embodiment comprises cyclosporin A, Nonatell 1123®, and Lemon oil.

Another embodiment comprises cyclosporin A, Nonatell 1153®, and Lemon oil.

Another embodiment comprises cyclosporin A, Nonatell 1161®, and Lemon oil.

Another embodiment comprises cyclosporin A, Nonatell 1172®, and Lemon oil.

Another embodiment comprises cyclosporin A, Nonatell 1181®, and Lemon oil.

Another embodiment comprises cyclosporin A, Norfox 36®, and Lemon oil.

Another embodiment comprises cyclosporin A, Procetyl AWS®, and Lemon oil.

Another embodiment comprises cyclosporin A, Sandoxylate SX 412®, and Lemon oil.

Another embodiment comprises cyclosporin A, Sandoxylate SX 418®, and Lemon oil.

Another embodiment comprises cyclosporin A, Surfonic JL-80X®, and Lemon oil.

Another embodiment comprises cyclosporin A, Surfonic JL-80X-B1®, and Lemon oil.

Another embodiment comprises cyclosporin A, Surfonic L4-29X®, and Lemon oil.

Another embodiment comprises cyclosporin A, Surfonic LF®, and Lemon oil.

Another embodiment comprises cyclosporin A, T-Det A826®, and Lemon oil.

Another embodiment comprises cyclosporin A, T-Det LF-416®, and Lemon oil.

Another embodiment comprises cyclosporin A, Tergitol Min-Foam 1X®, and Lemon oil.

Another embodiment comprises cyclosporin A, Tergitol Min-Foam 2X®, and Lemon oil.

Another embodiment comprises cyclosporin A, Triton CF-21®, and Lemon oil.

Another embodiment comprises cyclosporin A, Triton CF-76®, and Lemon oil.

Another embodiment comprises cyclosporin A, Triton XL-80N®, and Lemon oil.

Another embodiment comprises cyclosporin A, Witconol NS-98®, and Lemon oil.

Another embodiment comprises cyclosporin A, Witconol NS-108LQ®, and Lemon oil.

Another embodiment comprises cyclosporin A, Witconol NS-145®, and Lemon oil.

Another embodiment comprises cyclosporin A, Witconol NS-179®, and Lemon oil.

Another embodiment comprises cyclosporin A, a Protein-based Surfactant, and Lemon oil.

Another embodiment comprises cyclosporin A, AminoFoam W®, and Lemon oil.

Another embodiment comprises cyclosporin A, Amiter LGOD-2®, and Lemon oil.

Another embodiment comprises cyclosporin A, Amiter LGS-2®, and Lemon oil.

Another embodiment comprises cyclosporin A, Amiter LGS-5®, and Lemon oil.

Another embodiment comprises cyclosporin A, Lamepon S®, and Lemon oil.

Another embodiment comprises cyclosporin A, Lamepon S®, and Lemon oil.

Another embodiment comprises cyclosporin A, Maypon 4C®, and Lemon oil.

Another embodiment comprises cyclosporin A, May-Tein C®, and Lemon oil.

Another embodiment comprises cyclosporin A, May-Tein CT®, and Lemon oil.

Another embodiment comprises cyclosporin A, May-Tein KTS®, and Lemon oil.

Another embodiment comprises cyclosporin A, May-Tein SY®, and Lemon oil.

Another embodiment comprises cyclosporin A, Plantapon S®, and Lemon oil.

Another embodiment comprises cyclosporin A, Proteol APL®, and Lemon oil.

Another embodiment comprises cyclosporin A, Proteol OAT®, and Lemon oil.

Another embodiment comprises cyclosporin A, Pyroter CPI-40®, and Lemon oil.

Another embodiment comprises cyclosporin A, Pyroter GPI-25®, and Lemon oil.

Another embodiment comprises cyclosporin A, Supro-Tein S®, and Lemon oil.

Another embodiment comprises cyclosporin A, Supro-Tein V®, and Lemon oil.

Another embodiment comprises cyclosporin A,® and Lemon oil.

Another embodiment comprises cyclosporin A, a Sarcosine Derivative®, and Lemon oil.

Another embodiment comprises cyclosporin A, Crodasinic LS-30®, and Lemon oil.

Another embodiment comprises cyclosporin A, Vanseal CS®, and Lemon oil.

Another embodiment comprises cyclosporin A, Vanseal LS®, and Lemon oil.

Another embodiment comprises cyclosporin A, Vanseal MS®, and Lemon oil.

Another embodiment comprises cyclosporin A, Vanseal NACS-30®, and Lemon oil.

Another embodiment comprises cyclosporin A, Vanseal NALS-95®, and Lemon oil.

Another embodiment comprises cyclosporin A, Vanseal OS®, and Lemon oil.

Another embodiment comprises cyclosporin A,® and Lemon oil.

Another embodiment comprises cyclosporin A, a Silicone-based Surfactant®, and Lemon oil.

Another embodiment comprises cyclosporin A, Abil-B-9950®, and Lemon oil.

Another embodiment comprises cyclosporin A, Abil Care 858®, and Lemon oil.

Another embodiment comprises cyclosporin A, Abil EM 90®, and Lemon oil.

Another embodiment comprises cyclosporin A, Abil EM 97®, and Lemon oil.

Another embodiment comprises cyclosporin A, Abil WE-09®, and Lemon oil.

Another embodiment comprises cyclosporin A, Dow Corning 1248 Fluid®, and Lemon oil.

Another embodiment comprises cyclosporin A, Dow Corning 3225C®, and Lemon oil.

Another embodiment comprises cyclosporin A, Dow Corning 5200®, and Lemon oil.

Another embodiment comprises cyclosporin A, Dow Corning Q4-3667®, and Lemon oil.

Another embodiment comprises cyclosporin A, Monasil PCA®, and Lemon oil.

Another embodiment comprises cyclosporin A, Monasil PDM®, and Lemon oil.

Another embodiment comprises cyclosporin A, Monasil PLN®, and Lemon oil.

Another embodiment comprises cyclosporin A, Polyderm PPI-SI-WS®, and Lemon oil.

Another embodiment comprises cyclosporin A, Troysol 380W®, and Lemon oil.

Another embodiment comprises cyclosporin A, Troysol S366®, and Lemon oil.

Another embodiment comprises cyclosporin A, a Sorbitan Derivative, and Lemon oil.

Another embodiment comprises cyclosporin A, Alkamuls SML®, and Lemon oil.

Another embodiment comprises cyclosporin A, Alkamuls SMO®, and Lemon oil.

Another embodiment comprises cyclosporin A, Alkamuls STO®, and Lemon oil.

Another embodiment comprises cyclosporin A, Arlacel 20®, and Lemon oil.

Another embodiment comprises cyclosporin A, Arlacel 40®, and Lemon oil.

Another embodiment comprises cyclosporin A, Arlacel 60®, and Lemon oil.

Another embodiment comprises cyclosporin A, Arlacel 80®, and Lemon oil.

Another embodiment comprises cyclosporin A, Arlacel C®, and Lemon oil.

Another embodiment comprises cyclosporin A, Armul 21®, and Lemon oil.

Another embodiment comprises cyclosporin A, Atlox 80®, and Lemon oil.

Another embodiment comprises cyclosporin A, Atlox 847®, and Lemon oil.

Another embodiment comprises cyclosporin A, Atlox 1045A®, and Lemon oil.

Another embodiment comprises cyclosporin A, Canarcel 20®, and Lemon oil.

Another embodiment comprises cyclosporin A, Canarcel 60®, and Lemon oil.

Another embodiment comprises cyclosporin A, Canarcel 80®, and Lemon oil.

Another embodiment comprises cyclosporin A, Canarcel TW 20®, and Lemon oil.

Another embodiment comprises cyclosporin A, Canarcel TW 60®, and Lemon oil.

Another embodiment comprises cyclosporin A, Canarcel TW 80®, and Lemon oil.

Another embodiment comprises cyclosporin A, Coladet BSB-P®, and Lemon oil.

Another embodiment comprises cyclosporin A, Customulse O-20®, and Lemon oil.

Another embodiment comprises cyclosporin A, Dehymuls E®, and Lemon oil.

Another embodiment comprises cyclosporin A, DeSotan SMO®, and Lemon oil.

Another embodiment comprises cyclosporin A, DeSotan SMO-20®, and Lemon oil.

Another embodiment comprises cyclosporin A, DeSotan SMT®, and Lemon oil.

Another embodiment comprises cyclosporin A, DeSotan SMT-20®, and Lemon oil.

Another embodiment comprises cyclosporin A, Durfax 60®, and Lemon oil.

Another embodiment comprises cyclosporin A, Durfax 65®, and Lemon oil.

Another embodiment comprises cyclosporin A, Durfax 80®, and Lemon oil.

Another embodiment comprises cyclosporin A, Durtan 60®, and Lemon oil.

Another embodiment comprises cyclosporin A, Durtan 65®, and Lemon oil.

Another embodiment comprises cyclosporin A, Liposorb L®, and Lemon oil.

Another embodiment comprises cyclosporin A, Liposorb L-10®, and Lemon oil.

Another embodiment comprises cyclosporin A, Liposorb L-20®, and Lemon oil.

Another embodiment comprises cyclosporin A, Liposorb O®, and Lemon oil.

Another embodiment comprises cyclosporin A, Liposorb O-20®, and Lemon oil.

Another embodiment comprises cyclosporin A, Liposorb P®, and Lemon oil.

Another embodiment comprises cyclosporin A, Liposorb P-20®, and Lemon oil.

Another embodiment comprises cyclosporin A, Liposorb S®, and Lemon oil.

Another embodiment comprises cyclosporin A, Liposorb S-20®, and Lemon oil.

Another embodiment comprises cyclosporin A, Liposorb SQO®, and Lemon oil.

Another embodiment comprises cyclosporin A, Liposorb TO®, and Lemon oil.

Another embodiment comprises cyclosporin A, Liposorb TS®, and Lemon oil.

Another embodiment comprises cyclosporin A, Liposorb TS-20®, and Lemon oil.

Another embodiment comprises cyclosporin A, Lumisorb PS®, and Lemon oil.

Another embodiment comprises cyclosporin A, Lumisorb SMO (T)®, and Lemon oil.

Another embodiment comprises cyclosporin A, Lumisorb SMS K®, and Lemon oil.

Another embodiment comprises cyclosporin A, Lumisorb SSO®, and Lemon oil.

Another embodiment comprises cyclosporin A, Lumisorb STS K®, and Lemon oil.

Another embodiment comprises cyclosporin A, Lumisorb STT®, and Lemon oil.

Another embodiment comprises cyclosporin A, Magrabar SMO®, and Lemon oil.

Another embodiment comprises cyclosporin A, Magrabar SMO-VEG®, and Lemon oil.

Another embodiment comprises cyclosporin A, Magrabar SMT®, and Lemon oil.

Another embodiment comprises cyclosporin A, Magrabar STO®, and Lemon oil.

Another embodiment comprises cyclosporin A, Miracare BC-27®, and Lemon oil.

Another embodiment comprises cyclosporin A, Ritabate 20®, and Lemon oil.

Another embodiment comprises cyclosporin A, Ritabate 40®, and Lemon oil.

Another embodiment comprises cyclosporin A, Ritabate 60®, and Lemon oil.

Another embodiment comprises cyclosporin A, Ritabate 80®, and Lemon oil.

Another embodiment comprises cyclosporin A, T-Maz®, and Lemon oil.

Another embodiment comprises cyclosporin A, Tego SML®, and Lemon oil.

Another embodiment comprises cyclosporin A, Tego SML 20®, and Lemon oil.

Another embodiment comprises cyclosporin A, Tego SMO 80 V®, and Lemon oil.

Another embodiment comprises cyclosporin A, Tego SMO V®, and Lemon oil.

Another embodiment comprises cyclosporin A, Tego SMS®, and Lemon oil.

Another embodiment comprises cyclosporin A, Tego STO V®, and Lemon oil.

Another embodiment comprises cyclosporin A, Tween 21®, and Lemon oil.

Another embodiment comprises cyclosporin A, Tween 40®, and Lemon oil.

Another embodiment comprises cyclosporin A, Tween 60®, and Lemon oil.

Another embodiment comprises cyclosporin A, Tween 60 K®, and Lemon oil.

Another embodiment comprises cyclosporin A, Tween 61®, and Lemon oil.

Another embodiment comprises cyclosporin A, Tween 65®, and Lemon oil.

Another embodiment comprises cyclosporin A, Tween 80®, and Lemon oil.

Another embodiment comprises cyclosporin A, Tween 80 K®, and Lemon oil.

Another embodiment comprises cyclosporin A, Tween 81®, and Lemon oil.

Another embodiment comprises cyclosporin A, Tween 85®, and Lemon oil.

Another embodiment comprises cyclosporin A, a Sucrose or Glucose Ester, or Derivative thereof, and Lemon oil.

Another embodiment comprises cyclosporin A, DeSulf GOS-P-60WCG®, and Lemon oil.

Another embodiment comprises cyclosporin A, Glucam E-20 Distearate®, and Lemon oil.

Another embodiment comprises cyclosporin A, Glucamate DOE-120®, and Lemon oil.

Another embodiment comprises cyclosporin A, Glucamate SSE-20®, and Lemon oil.

Another embodiment comprises cyclosporin A, Glucate DO®, and Lemon oil.

Another embodiment comprises cyclosporin A, Glucate SS®, and Lemon oil.

Another embodiment comprises cyclosporin A, Glucopon 425 UP®, and Lemon oil.

Another embodiment comprises cyclosporin A, Isolan IS®, and Lemon oil.

Another embodiment comprises cyclosporin A, Mazon 40®, and Lemon oil.

Another embodiment comprises cyclosporin A, Montanov 82®, and Lemon oil.

Another embodiment comprises cyclosporin A, Montanov 202®, and Lemon. oil.

Another embodiment comprises cyclosporin A, Montanov S®, and Lemon oil.

Another embodiment comprises cyclosporin A, Rheozan®, and Lemon oil.

Another embodiment comprises cyclosporin A, Simulsol AS 48®, and Lemon oil.

Another embodiment comprises cyclosporin A, Simulsol SL 4®, and Lemon oil.

Another embodiment comprises cyclosporin A, Simulsol SL 10®, and Lemon oil.

Another embodiment comprises cyclosporin A, Simulsol SL 11W®, and Lemon oil.

Another embodiment comprises cyclosporin A, Simulsol SL 55®, and Lemon oil.

Another embodiment comprises cyclosporin A, Suga Nate 100 and 160®, and Lemon oil.

Another embodiment comprises cyclosporin A, Tego Care 450®, and Lemon oil.

Another embodiment comprises cyclosporin A, Tego Care CG 90®, and Lemon oil.

Another embodiment comprises cyclosporin A, Tego Care PS®, and Lemon oil.

Another embodiment comprises cyclosporin A, Tegosoft PSE 141 G®, and Lemon oil.

Another embodiment comprises cyclosporin A, Tegotens G 826®, and Lemon oil.

Another embodiment comprises cyclosporin A, Triton BG-10 (70%)®, and Lemon oil.

Another embodiment comprises cyclosporin A, Triton CG-110 (60%)®, and Lemon oil.

Another embodiment comprises cyclosporin A, Wickenol 545®, and Lemon oil.

Another embodiment comprises cyclosporin A, an alcohol, and Orange oil.

Another embodiment comprises cyclosporin A, Diglycerol®, and Orange oil.

Another embodiment comprises cyclosporin A, Hetoxide GT-80®, and Orange oil.

Another embodiment comprises cyclosporin A, Lexemul BEO®, and Orange oil.

Another embodiment comprises cyclosporin A, Polyglycerol-3®, and Orange oil.

Another embodiment comprises cyclosporin A, Redicote E Series®, and Orange oil.

Another embodiment comprises cyclosporin A, Simulsol OX 1005L®, and Orange oil.

Another embodiment comprises cyclosporin A, Stanfax 567®, and Orange oil.

Another embodiment comprises cyclosporin A, TA-1618®, and Orange oil.

Another embodiment comprises cyclosporin A, Witconol H-31A®, and Orange oil.

Another embodiment comprises cyclosporin A, an amine oxide, and Orange oil.

Another embodiment comprises cyclosporin A, AO-405®, and Orange oil.

Another embodiment comprises cyclosporin A, AO-455®, and Orange oil.

Another embodiment comprises cyclosporin A, AO 728 Special®, and Orange oil.

Another embodiment comprises cyclosporin A, Barlox 12®, and Orange oil.

Another embodiment comprises cyclosporin A, Barlox 14®, and Orange oil.

Another embodiment comprises cyclosporin A, Burcoxide Lo®, and Orange oil.

Another embodiment comprises cyclosporin A, Caloxamine LO®, and Orange oil.

Another embodiment comprises cyclosporin A, Chemoxide CAW®, and Orange oil.

Another embodiment comprises cyclosporin A, Chemoxide LM-30®, and Orange oil.

Another embodiment comprises cyclosporin A, Chemoxide LO®, and Orange oil.

Another embodiment comprises cyclosporin A, Chemoxide MO®, and Orange oil.

Another embodiment comprises cyclosporin A, Colalux CAO-35®, and Orange oil.

Another embodiment comprises cyclosporin A, Colalux LO®, and Orange oil.

Another embodiment comprises cyclosporin A, DeMox CAPO®, and Orange oil.

Another embodiment comprises cyclosporin A, DeMox CSG-30®, and Orange oil.

Another embodiment comprises cyclosporin A, DeMox LAO®, and Orange oil.

Another embodiment comprises cyclosporin A, Emcol L®, and Orange oil.

Another embodiment comprises cyclosporin A, Empigen OB®, and Orange oil.

Another embodiment comprises cyclosporin A, Empigen OS/A®, and Orange oil.

Another embodiment comprises cyclosporin A, Foamox CDO®, and Orange oil.

Another embodiment comprises cyclosporin A, Foamox DMM®, and Orange oil.

Another embodiment comprises cyclosporin A, Foamox DMS®, and Orange oil.

Another embodiment comprises cyclosporin A, Genaminox KC®, and Orange oil.

Another embodiment comprises cyclosporin A, Genaminox LA®, and Orange oil.

Another embodiment comprises cyclosporin A, Hartofoam SAO®, and Orange oil.

Another embodiment comprises cyclosporin A, Hartox DMCD®, and Orange oil.

Another embodiment comprises cyclosporin A, Lipowax DAT®, and Orange oil.

Another embodiment comprises cyclosporin A, Lipowax PB Pastilles®, and Orange oil.

Another embodiment comprises cyclosporin A, Mackamine C8®, and Orange oil.

Another embodiment comprises cyclosporin A, Mackamine C10®, and Orange oil.

Another embodiment comprises cyclosporin A, Mackamine C14®, and Orange oil.

Another embodiment comprises cyclosporin A, Mackamine CAO®, and Orange oil.

Another embodiment comprises cyclosporin A, Mackamine CO®, and Orange oil.

Another embodiment comprises cyclosporin A, Mackamine LO®, and Orange oil.

Another embodiment comprises cyclosporin A, Mackamine O2®, and Orange oil.

Another embodiment comprises cyclosporin A, Mackamine SAO®, and Orange oil.

Another embodiment comprises cyclosporin A, Mackamine SO®, and Orange oil.

Another embodiment comprises cyclosporin A, Mazox KCAO®, and Orange oil.

Another embodiment comprises cyclosporin A, Monalac MO®, and Orange oil.

Another embodiment comprises cyclosporin A, Norfox LDA®, and Orange oil.

Another embodiment comprises cyclosporin A, Rhodamox LO®, and Orange oil.

Another embodiment comprises cyclosporin A, Schercamox C-AA®, and Orange oil.

Another embodiment comprises cyclosporin A, Schercamox DMA®, and Orange oil.

Another embodiment comprises cyclosporin A, Schercamox DML®, and Orange oil.

Another embodiment comprises cyclosporin A, Schercamox DMM®, and Orange oil.

Another embodiment comprises cyclosporin A, Schercamox DMS®, and Orange oil.

Another embodiment comprises cyclosporin A, Tegotens DO®, and Orange oil.

Another embodiment comprises cyclosporin A, Tomah AO-14-2®, and Orange oil.

Another embodiment comprises cyclosporin A, Triaminox CDO®, and Orange oil.

Another embodiment comprises cyclosporin A, a block polymer, and Orange oil.

Another embodiment comprises cyclosporin A, AL 2070®, and Orange oil.

Another embodiment comprises cyclosporin A, Antarox 17-R-2®, and Orange oil.

Another embodiment comprises cyclosporin A, Antarox 25-R-2®, and Orange oil.

Another embodiment comprises cyclosporin A, Antarox 31-R-1®, and Orange oil.

Another embodiment comprises cyclosporin A, Antarox P-84®, and Orange oil.

Another embodiment comprises cyclosporin A, Antarox P-104/H®, and Orange oil.

Another embodiment comprises cyclosporin A, Arnox BP-Series®, and Orange oil.

Another embodiment comprises cyclosporin A, Chemonic 435®, and Orange oil.

Another embodiment comprises cyclosporin A, Chemonic D-25®, and Orange oil.

Another embodiment comprises cyclosporin A, Chemonic PL Series®, and Orange oil.

Another embodiment comprises cyclosporin A, Ethox L-121®, and Orange oil.

Another embodiment comprises cyclosporin A, Ethox L-122®, and Orange oil.

Another embodiment comprises cyclosporin A, Genapol PF-10®, and Orange oil.

Another embodiment comprises cyclosporin A, Genapol PF-20®, and Orange oil.

Another embodiment comprises cyclosporin A, Genapol PF-40A®, and Orange oil.

Another embodiment comprises cyclosporin A, Norfox 2-LF®, and Orange oil.

Another embodiment comprises cyclosporin A, Simulsol NW 342®, and Orange oil.

Another embodiment comprises cyclosporin A, T-Det BP-1®, and Orange oil.

Another embodiment comprises cyclosporin A, T-Det XD®, and Orange oil.

Another embodiment comprises cyclosporin A, T-Det XH®, and Orange oil.

Another embodiment comprises cyclosporin A, Triton CF-32®, and Orange oil.

Another embodiment comprises cyclosporin A, Witconol 171®, and Orange oil.

Another embodiment comprises cyclosporin A, Witconol 324®, and Orange oil.

Another embodiment comprises cyclosporin A, Witconol 324D®, and Orange oil.

Another embodiment comprises cyclosporin A, Witconol PD-2000®, and Orange oil.

Another embodiment comprises cyclosporin A, a Carboxylated Alcohol or Alkylphenol Ethoxylate, and Orange oil.

Another embodiment comprises cyclosporin A, Emcol CN-6®, and Orange oil.

Another embodiment comprises cyclosporin A, Ethcarb®, and Orange oil.

Another embodiment comprises cyclosporin A, Gemtex WNT-Conc®, and Orange oil.

Another embodiment comprises cyclosporin A, Incrodet TD7-C®, and Orange oil.

Another embodiment comprises cyclosporin A, Marlinat CM 105/80®, and Orange oil.

Another embodiment comprises cyclosporin A, Marlowet 1072®, and Orange oil.

Another embodiment comprises cyclosporin A, Marlowet 4530®, and Orange oil.

Another embodiment comprises cyclosporin A, Marlowet 4530 LF®, and Orange oil.

Another embodiment comprises cyclosporin A, Marlowet 4534®, and Orange oil.

Another embodiment comprises cyclosporin A, Marlowet 4538®, and Orange oil.

Another embodiment comprises cyclosporin A, Marlowet 4539®, and Orange oil.

Another embodiment comprises cyclosporin A, Marlowet 4539 LF®, and Orange oil.

Another embodiment comprises cyclosporin A, Marlowet 4541®, and Orange oil.

Another embodiment comprises cyclosporin A, Miranate LEC-80®, and Orange oil.

Another embodiment comprises cyclosporin A, Sandopan B®, and Orange oil.

Another embodiment comprises cyclosporin A, Sandopan B Modified®, and Orange oil.

Another embodiment comprises cyclosporin A, Sandopan LS-24 Gel®, and Orange oil.

Another embodiment comprises cyclosporin A, Surfine T-A®, and Orange oil.

Another embodiment comprises cyclosporin A, a Carboxylic Acid/Fatty Acid, and Orange oil.

Another embodiment comprises cyclosporin A, Colaterge RAM®, and Orange oil.

Another embodiment comprises cyclosporin A, Colatrope INC®, and Orange oil.

Another embodiment comprises cyclosporin A, Crodacid B®, and Orange oil.

Another embodiment comprises cyclosporin A, DeTrope CA-100®, and Orange oil.

Another embodiment comprises cyclosporin A, Latol MTO®, and Orange oil.

Another embodiment comprises cyclosporin A, Lumulse CC-33 K®, and Orange oil.

Another embodiment comprises cyclosporin A, Mulls 2218®, and Orange oil.

Another embodiment comprises cyclosporin A, OL-600®, and Orange oil.

Another embodiment comprises cyclosporin A, OL-800®, and Orange oil.

Another embodiment comprises cyclosporin A, R-910®, and Orange oil.

Another embodiment comprises cyclosporin A, S-210®, and Orange oil.

Another embodiment comprises cyclosporin A, Sandopan DTC Acid®, and Orange oil.

Another embodiment comprises cyclosporin A, Sandopan LS 24 N®, and Orange oil.

Another embodiment comprises cyclosporin A, Sandopan MA-18®, and Orange oil.

Another embodiment comprises cyclosporin A, an Ethoxylated Alcohol, and Orange oil.

Another embodiment comprises cyclosporin A, Adsee 799®, and Orange oil.

Another embodiment comprises cyclosporin A, Adsee 799®, and Orange oil.

Another embodiment comprises cyclosporin A, Alfonic 610-3.5®, and Orange oil.

Another embodiment comprises cyclosporin A, Alfonic 810-2®, and Orange oil.

Another embodiment comprises cyclosporin A, Alfonic 810-6®, and Orange oil.

Another embodiment comprises cyclosporin A, Alfonic 1012-3®, and Orange oil.

Another embodiment comprises cyclosporin A, Alfonic 1012-5®, and Orange oil.

Another embodiment comprises cyclosporin A, Alfonic 1216CO-1.5®, and Orange oil.

Another embodiment comprises cyclosporin A, Alfonic 1216CO-7®, and Orange oil.

Another embodiment comprises cyclosporin A, Alfonic 1412-3®, and Orange oil.

Another embodiment comprises cyclosporin A, Alfonic 1412-7®, and Orange oil.

Another embodiment comprises cyclosporin A, Arlasolve 200®, and Orange oil.

Another embodiment comprises cyclosporin A, Arlasolve 200 Liquid®, and Orange oil.

Another embodiment comprises cyclosporin A, Armix 180-C®, and Orange oil.

Another embodiment comprises cyclosporin A, Armix 183®, and Orange oil.

Another embodiment comprises cyclosporin A, Armul 2404®, and Orange oil.

Another embodiment comprises cyclosporin A, Armul 2404®, and Orange oil.

Another embodiment comprises cyclosporin A, Atlas EMJ-C®, and Orange oil.

Another embodiment comprises cyclosporin A, Atlas G-2109®, and Orange oil.

Another embodiment comprises cyclosporin A, Atlas G-3886®, and Orange oil.

Another embodiment comprises cyclosporin A, Atlas G-3890®, and Orange oil.

Another embodiment comprises cyclosporin A, Bio Soft E-200®, and Orange oil.

Another embodiment comprises cyclosporin A, Bio Soft E-300®, and Orange oil.

Another embodiment comprises cyclosporin A, Bio Soft E-400®, and Orange oil.

Another embodiment comprises cyclosporin A, Bio Soft EN 600®, and Orange oil.

Another embodiment comprises cyclosporin A, Bio Soft TD-400®, and Orange oil.

Another embodiment comprises cyclosporin A, Bio Soft TD-630®, and Orange oil.

Another embodiment comprises cyclosporin A, Brij 30®, and Orange oil.

Another embodiment comprises cyclosporin A, Brij 52®, and Orange oil.

Another embodiment comprises cyclosporin A, Brij 56®, and Orange oil.

Another embodiment comprises cyclosporin A, Brij 58®, and Orange oil.

Another embodiment comprises cyclosporin A, Brij 72®, and Orange oil.

Another embodiment comprises cyclosporin A, Brij 76®, and Orange oil.

Another embodiment comprises cyclosporin A, Brij 78®, and Orange oil.

Another embodiment comprises cyclosporin A, Brij 93®, and Orange oil.

Another embodiment comprises cyclosporin A, Brij 97®, and Orange oil.

Another embodiment comprises cyclosporin A, Brij 98®, and Orange oil.

Another embodiment comprises cyclosporin A, Brij 700®, and Orange oil.

Another embodiment comprises cyclosporin A, Brij 700 S®, and Orange oil.

Another embodiment comprises cyclosporin A, Brij 721®, and Orange oil.

Another embodiment comprises cyclosporin A, Brij 721 S®, and Orange oil.

Another embodiment comprises cyclosporin A, Burcoterge CDG®, and Orange oil.

Another embodiment comprises cyclosporin A, Canasol AT 600®, and Orange oil.

Another embodiment comprises cyclosporin A, Canasol AT 800®, and Orange oil.

Another embodiment comprises cyclosporin A, Canasol AT 1200®, and Orange oil.

Another embodiment comprises cyclosporin A, Canasol BJ 35®, and Orange oil.

Another embodiment comprises cyclosporin A, Canasol BJ 36®, and Orange oil.

Another embodiment comprises cyclosporin A, Canasol BJ 52®, and Orange oil.

Another embodiment comprises cyclosporin A, Canasol BJ 58®, and Orange oil.

Another embodiment comprises cyclosporin A, Canasol BJ 72®, and Orange oil.

Another embodiment comprises cyclosporin A, Canasol BJ 78®, and Orange oil.

Another embodiment comprises cyclosporin A, Canasol BJ 98®, and Orange oil.

Another embodiment comprises cyclosporin A, Canasol BJ 307®, and Orange oil.

Another embodiment comprises cyclosporin A, Cerfak 1400®, and Orange oil.

Another embodiment comprises cyclosporin A, Cetomacrogol 1000 BP®, and Orange oil.

Another embodiment comprises cyclosporin A, Chemonic C-2®, and Orange oil.

Another embodiment comprises cyclosporin A, Chemonic C-10®, and Orange oil.

Another embodiment comprises cyclosporin A, Chemonic C-20®, and Orange oil.

Another embodiment comprises cyclosporin A, Chemonic CT-12®, and Orange oil.

Another embodiment comprises cyclosporin A, Chemonic CT-20®, and Orange oil.

Another embodiment comprises cyclosporin A, Chemonic CT-30®, and Orange oil.

Another embodiment comprises cyclosporin A, Chemonic CT-55®, and Orange oil.

Another embodiment comprises cyclosporin A, Chemonic G-7®, and Orange oil.

Another embodiment comprises cyclosporin A, Chemonic G-26®, and Orange oil.

Another embodiment comprises cyclosporin A, Chemonic L-4®, and Orange oil.

Another embodiment comprises cyclosporin A, Chemonic L-7®, and Orange oil.

Another embodiment comprises cyclosporin A, Chemonic L-12®, and Orange oil.

Another embodiment comprises cyclosporin A, Chemonic L-23®, and Orange oil.

Another embodiment comprises cyclosporin A, Chemonic O-2®, and Orange oil.

Another embodiment comprises cyclosporin A, Chemonic O-5®, and Orange oil.

Another embodiment comprises cyclosporin A, Chemonic O-10®, and Orange oil.

Another embodiment comprises cyclosporin A, Chemonic O-20®, and Orange oil.

Another embodiment comprises cyclosporin A, Chemonic S-2®, and Orange oil.

Another embodiment comprises cyclosporin A, Chemonic S-10®, and Orange oil.

Another embodiment comprises cyclosporin A, Chemonic S-20®, and Orange oil.

Another embodiment comprises cyclosporin A, Colamulse FE®, and Orange oil.

Another embodiment comprises cyclosporin A, Cremophor A 20®, and Orange oil.

Another embodiment comprises cyclosporin A, Cremophor SA 2®, and Orange oil.

Another embodiment comprises cyclosporin A, Dehydol 100®, and Orange oil.

Another embodiment comprises cyclosporin A, Dehydol O-4®, and Orange oil.

Another embodiment comprises cyclosporin A, Delonic C-18®, and Orange oil.

Another embodiment comprises cyclosporin A, DeSonic 6T®, and Orange oil.

Another embodiment comprises cyclosporin A, DeSonic 9D®, and Orange oil.

Another embodiment comprises cyclosporin A, DeSonic 9T®, and Orange oil.

Another embodiment comprises cyclosporin A, DeSonic 12D®, and Orange oil.

Another embodiment comprises cyclosporin A, DeSonic 12T®, and Orange oil.

Another embodiment comprises cyclosporin A, DeSonic 15T®, and Orange oil.

Another embodiment comprises cyclosporin A, DeSonic TDA-9®, and Orange oil.

Another embodiment comprises cyclosporin A, DeThox GLG-7®, and Orange oil.

Another embodiment comprises cyclosporin A, DeThox GLG-26®, and Orange oil.

Another embodiment comprises cyclosporin A, DeThox LA-4®, and Orange oil.

Another embodiment comprises cyclosporin A, DeThox LA-23®, and Orange oil.

Another embodiment comprises cyclosporin A, DeThox SA-80®, and Orange oil.

Another embodiment comprises cyclosporin A, Disponil O5®, and Orange oil.

Another embodiment comprises cyclosporin A, Eccoterge EO-41B®, and Orange oil.

Another embodiment comprises cyclosporin A, Empilan KA2.5/90®, and Orange oil.

Another embodiment comprises cyclosporin A, Empilan KA5/90®, and Orange oil.

Another embodiment comprises cyclosporin A, Empilan KM-20®, and Orange oil.

Another embodiment comprises cyclosporin A, Empilan KM-50®, and Orange oil.

Another embodiment comprises cyclosporin A, Empilan L-23®, and Orange oil.

Another embodiment comprises cyclosporin A, Ethylan 25-3®, and Orange oil.

Another embodiment comprises cyclosporin A, Ethylan 1204®, and Orange oil.

Another embodiment comprises cyclosporin A, Ethylan DA-4®, and Orange oil.

Another embodiment comprises cyclosporin A, Ethylan LA-230®, and Orange oil.

Another embodiment comprises cyclosporin A, Ethylan SN®, and Orange oil.

Another embodiment comprises cyclosporin A, Ethylan TD-60®, and Orange oil.

Another embodiment comprises cyclosporin A, Ethylan TD-100®, and Orange oil.

Another embodiment comprises cyclosporin A, Ethylan TD-1407®, and Orange oil.

Another embodiment comprises cyclosporin A, Eumulgin B1®, and Orange oil.

Another embodiment comprises cyclosporin A, Eumulgin B2®, and Orange oil.

Another embodiment comprises cyclosporin A, Eumulgin B3®, and Orange oil.

Another embodiment comprises cyclosporin A, Eumulgin O-10®, and Orange oil.

Another embodiment comprises cyclosporin A, Flo Mo 80/20®, and Orange oil.

Another embodiment comprises cyclosporin A, Flo Mo Low Foam®, and Orange oil.

Another embodiment comprises cyclosporin A, Forlan C-24®, and Orange oil.

Another embodiment comprises cyclosporin A, Genapol 1454®, and Orange oil.

Another embodiment comprises cyclosporin A, Genapol BA-020®, and Orange oil.

Another embodiment comprises cyclosporin A, Genapol BA-040®, and Orange oil.

Another embodiment comprises cyclosporin A, Genapol C-100®, and Orange oil.

Another embodiment comprises cyclosporin A, Genapol DA 060®, and Orange oil.

Another embodiment comprises cyclosporin A, Genapol HS 020®, and Orange oil.

Another embodiment comprises cyclosporin A, Genapol HS 200®, and Orange oil.

Another embodiment comprises cyclosporin A, Genapol ID-040®, and Orange oil.

Another embodiment comprises cyclosporin A, Genapol ID-060®, and Orange oil.

Another embodiment comprises cyclosporin A, Genapol ID-090®, and Orange oil.

Another embodiment comprises cyclosporin A, Genapol LA 010®, and Orange oil.

Another embodiment comprises cyclosporin A, Genapol LA 020®, and Orange oil.

Another embodiment comprises cyclosporin A, Genapol LA 030®, and Orange oil.

Another embodiment comprises cyclosporin A, Genapol LA 040®, and Orange oil.

Another embodiment comprises cyclosporin A, Genapol LA 050®, and Orange oil.

Another embodiment comprises cyclosporin A, Genapol LA 060®, and Orange oil.

Another embodiment comprises cyclosporin A, Genapol LA 070®, and Orange oil.

Another embodiment comprises cyclosporin A, Genapol LA 070S®, and Orange oil.

Another embodiment comprises cyclosporin A, Genapol LA 230®, and Orange oil.

Another embodiment comprises cyclosporin A, Genapol O 020®, and Orange oil.

Another embodiment comprises cyclosporin A, Genapol O 050®, and Orange oil.

Another embodiment comprises cyclosporin A, Genapol O 100®, and Orange oil.

Another embodiment comprises cyclosporin A, Genapol O 200®, and Orange oil.

Another embodiment comprises cyclosporin A, Genapol SA 030®, and Orange oil.

Another embodiment comprises cyclosporin A, Genapol SA 120®, and Orange oil.

Another embodiment comprises cyclosporin A, Genapol T-020®, and Orange oil.

Another embodiment comprises cyclosporin A, Genapol UD-030®, and Orange oil.

Another embodiment comprises cyclosporin A, Genapol UD-050®, and Orange oil.

Another embodiment comprises cyclosporin A, Genapol UD-070®, and Orange oil.

Another embodiment comprises cyclosporin A, Genapol UD-079®, and Orange oil.

Another embodiment comprises cyclosporin A, Genapol UD-080®, and Orange oil.

Another embodiment comprises cyclosporin A, Genapol UD-110®, and Orange oil.

Another embodiment comprises cyclosporin A, Genapol X 030®, and Orange oil.

Another embodiment comprises cyclosporin A, Genapol X 050®, and Orange oil.

Another embodiment comprises cyclosporin A, Genapol X 060®, and Orange oil.

Another embodiment comprises cyclosporin A, Genapol X 070®, and Orange oil.

Another embodiment comprises cyclosporin A, Genapol X 080®, and Orange oil.

Another embodiment comprises cyclosporin A, Genapol X 100®, and Orange oil.

Another embodiment comprises cyclosporin A, Genapol X159®, and Orange oil.

Another embodiment comprises cyclosporin A, Generol 122 E5®, and Orange oil.

Another embodiment comprises cyclosporin A, Generol 122 E25®, and Orange oil.

Another embodiment comprises cyclosporin A, Hostacerin T-3®, and Orange oil.

Another embodiment comprises cyclosporin A, Iconol LF 110®, and Orange oil.

Another embodiment comprises cyclosporin A, Incropol CS-20®, and Orange oil.

Another embodiment comprises cyclosporin A, Lexemul CS-20®, and Orange oil.

Another embodiment comprises cyclosporin A, Liponic EG-1®, and Orange oil.

Another embodiment comprises cyclosporin A, Lipowax D®, and Orange oil.

Another embodiment comprises cyclosporin A, Lipowax G®, and Orange oil.

Another embodiment comprises cyclosporin A, Lipowax NI®, and Orange oil.

Another embodiment comprises cyclosporin A, Lipowax P®, and Orange oil.

Another embodiment comprises cyclosporin A, Lipowax P-31®, and Orange oil.

Another embodiment comprises cyclosporin A, Lipowax PR®, and Orange oil.

Another embodiment comprises cyclosporin A, Lumulse CS-20®, and Orange oil.

Another embodiment comprises cyclosporin A, Macol CSA-20®, and Orange oil.

Another embodiment comprises cyclosporin A, Marlox B 24/50®, and Orange oil.

Another embodiment comprises cyclosporin A, Mazawet 77®, and Orange oil.

Another embodiment comprises cyclosporin A, Norfox 1713®, and Orange oil.

Another embodiment comprises cyclosporin A, Norfox 2579®, and Orange oil.

Another embodiment comprises cyclosporin A, Norfox Lo Foam®, and Orange oil.

Another embodiment comprises cyclosporin A, Promulgen D®, and Orange oil.

Another embodiment comprises cyclosporin A, Promulgen G®, and Orange oil.

Another embodiment comprises cyclosporin A, Renex 30®, and Orange oil.

Another embodiment comprises cyclosporin A, Renex 36®, and Orange oil.

Another embodiment comprises cyclosporin A, Rhodasurf A 24®, and Orange oil.

Another embodiment comprises cyclosporin A, Rhodasurf AAE-10®, and Orange oil.

Another embodiment comprises cyclosporin A, Rhodasurf BEH-25®, and Orange oil.

Another embodiment comprises cyclosporin A, Rhodasurf BEH-40®, and Orange oil.

Another embodiment comprises cyclosporin A, Rhodasurf DA 530®, and Orange oil.

Another embodiment comprises cyclosporin A, Rhodasurf DA 630®, and Orange oil.

Another embodiment comprises cyclosporin A, Rhodasurf DA 639®, and Orange oil.

Another embodiment comprises cyclosporin A, Rhodasurf LAN-23®, and Orange oil.

Another embodiment comprises cyclosporin A, Rhodasurf ON-870®, and Orange oil.

Another embodiment comprises cyclosporin A, Rhodasurf ON-877®, and Orange oil.

Another embodiment comprises cyclosporin A, Rhodasurf TB-970 FLK®, and Orange oil.

Another embodiment comprises cyclosporin A, Ritacet-20®, and Orange oil.

Another embodiment comprises cyclosporin A, Ritachol 1000®, and Orange oil.

Another embodiment comprises cyclosporin A, Ritachol 2000®, and Orange oil.

Another embodiment comprises cyclosporin A, Ritachol 5000®, and Orange oil.

Another embodiment comprises cyclosporin A, Ritox 35®, and Orange oil.

Another embodiment comprises cyclosporin A, Surfonic DA-4®, and Orange oil.

Another embodiment comprises cyclosporin A, Surfonic DA-6®, and Orange oil.

Another embodiment comprises cyclosporin A, Surfonic L46-7®, and Orange oil.

Another embodiment comprises cyclosporin A, Surfonic POA®, and Orange oil.

Another embodiment comprises cyclosporin A, Synthrapol KB®, and Orange oil.

Another embodiment comprises cyclosporin A, Teginacid®, and Orange oil.

Another embodiment comprises cyclosporin A, Teginacid C®, and Orange oil.

Another embodiment comprises cyclosporin A, Tegotens EC 11®, and Orange oil.

Another embodiment comprises cyclosporin A, Tinegal NA®, and Orange oil.

Another embodiment comprises cyclosporin A, Tomadol 400®, and Orange oil.

Another embodiment comprises cyclosporin A, Tomadol 600®, and Orange oil.

Another embodiment comprises cyclosporin A, Tomadol 900®, and Orange oil.

Another embodiment comprises cyclosporin A, Uniperol O®, and Orange oil.

Another embodiment comprises cyclosporin A, Witconol SN Series®, and Orange oil.

Another embodiment comprises cyclosporin A, an Ethoxylated Alkylphenol, and Orange oil.

Another embodiment comprises cyclosporin A, Antarox LF-222®, and Orange oil.

Another embodiment comprises cyclosporin A, Atlox 775®, and Orange oil.

Another embodiment comprises cyclosporin A, Caloxylate N-9®, and Orange oil.

Another embodiment comprises cyclosporin A, Canasol NF-1000®, and Orange oil.

Another embodiment comprises cyclosporin A, Canasol NF-3000®, and Orange oil.

Another embodiment comprises cyclosporin A, Canasol NF-3070®, and Orange oil.

Another embodiment comprises cyclosporin A, Canasol OF 1670®, and Orange oil.

Another embodiment comprises cyclosporin A, Canasol OF 2570®, and Orange oil.

Another embodiment comprises cyclosporin A, Canasol OF 4070®, and Orange oil.

Another embodiment comprises cyclosporin A, Chemax DNP-8®, and Orange oil.

Another embodiment comprises cyclosporin A, Chemax DNP-18®, and Orange oil.

Another embodiment comprises cyclosporin A, Chemax DNP-150/50®, and Orange oil.

Another embodiment comprises cyclosporin A, DeSonic 1.5N®, and Orange oil.

Another embodiment comprises cyclosporin A, DeSonic 4N®, and Orange oil.

Another embodiment comprises cyclosporin A, DeSonic 5N®, and Orange oil.

Another embodiment comprises cyclosporin A, DeSonic 6D®, and Orange oil.

Another embodiment comprises cyclosporin A, DeSonic 6N®, and Orange oil.

Another embodiment comprises cyclosporin A, DeSonic 7N®, and Orange oil.

Another embodiment comprises cyclosporin A, DeSonic 9N®, and Orange oil.

Another embodiment comprises cyclosporin A, DeSonic 10D®, and Orange oil.

Another embodiment comprises cyclosporin A, DeSonic 11N®, and Orange oil.

Another embodiment comprises cyclosporin A, DeSonic 12N®, and Orange oil.

Another embodiment comprises cyclosporin A, DeSonic 15N®, and Orange oil.

Another embodiment comprises cyclosporin A, DeSonic 20N®, and Orange oil.

Another embodiment comprises cyclosporin A, Eccoscour RC®, and Orange oil.

Another embodiment comprises cyclosporin A, Eccoterge EO-100®, and Orange oil.

Another embodiment comprises cyclosporin A, Emulsifier 632/90%®, and Orange oil.

Another embodiment comprises cyclosporin A, Geronol AG-821®, and Orange oil.

Another embodiment comprises cyclosporin A, Gradonic N-95®, and Orange oil.

Another embodiment comprises cyclosporin A, Hetoxide NP-4®, and Orange oil.

Another embodiment comprises cyclosporin A, Hetoxide NP-30®, and Orange oil.

Another embodiment comprises cyclosporin A, Hostapal N-100®, and Orange oil.

Another embodiment comprises cyclosporin A, Hostapal N-110®, and Orange oil.

Another embodiment comprises cyclosporin A, Igepal CTA-639W®, and Orange oil.

Another embodiment comprises cyclosporin A, Igepal DAP-9®, and Orange oil.

Another embodiment comprises cyclosporin A, Igepal OD-410®, and Orange oil.

Another embodiment comprises cyclosporin A, Igepal SS-837®, and Orange oil.

Another embodiment comprises cyclosporin A, Lipocol NP-9 USP®, and Orange oil.

Another embodiment comprises cyclosporin A, Macol DNP-10®, and Orange oil.

Another embodiment comprises cyclosporin A, Marlophen NP 5®, and Orange oil.

Another embodiment comprises cyclosporin A, Marlophen P 1®, and Orange oil.

Another embodiment comprises cyclosporin A, Surfonic NB®, and Orange oil.

Another embodiment comprises cyclosporin A, Surfonic OPB-307®, and Orange oil.

Another embodiment comprises cyclosporin A, Surfonic OPB-407®, and Orange oil.

Another embodiment comprises cyclosporin A, Syn Fac 334®, and Orange oil.

Another embodiment comprises cyclosporin A, Syn Fac 8216®, and Orange oil.

Another embodiment comprises cyclosporin A, Triton N-57®, and Orange oil.

Another embodiment comprises cyclosporin A, Trycol 6956®, and Orange oil.

Another embodiment comprises cyclosporin A, Trycol 6961®, and Orange oil.

Another embodiment comprises cyclosporin A, Trycol 6964®, and Orange oil.

Another embodiment comprises cyclosporin A, Trycol 6969®, and Orange oil.

Another embodiment comprises cyclosporin A, Trycol 6974®, and Orange oil.

Another embodiment comprises cyclosporin A, Witbreak DRB-127®, and Orange oil.

Another embodiment comprises cyclosporin A, Witbreak DRB-127®, and Orange oil.

Another embodiment comprises cyclosporin A, Witconol NP Series®, and Orange oil.

Another embodiment comprises cyclosporin A, an Ethoxylated Aryl Phenol, and Orange oil.

Another embodiment comprises cyclosporin A, Soprophor BSU®, and Orange oil.

Another embodiment comprises cyclosporin A, Soprophor CY/8®, and Orange oil.

Another embodiment comprises cyclosporin A, Soprophor S/25®, and Orange oil.

Another embodiment comprises cyclosporin A, Witconol NIO®, and Orange oil.

Another embodiment comprises cyclosporin A, Witconol NIW®, and Orange oil.

Another embodiment comprises cyclosporin A, Witconol S-100®, and Orange oil.

Another embodiment comprises cyclosporin A, an Ethoxylated Fatty Acid, and Orange oil.

Another embodiment comprises cyclosporin A, Aldo PGHMS®, and Orange oil.

Another embodiment comprises cyclosporin A, Alkamuls TO-15/HR®, and Orange oil.

Another embodiment comprises cyclosporin A, Armotan AL-69-66®, and Orange oil.

Another embodiment comprises cyclosporin A, Cerasynt 840®, and Orange oil.

Another embodiment comprises cyclosporin A, Cerasynt 945®, and Orange oil.

Another embodiment comprises cyclosporin A, Crystal Inhibitor No. 5®, and Orange oil.

Another embodiment comprises cyclosporin A, DeThox Acid L-9®, and Orange oil.

Another embodiment comprises cyclosporin A, DeThox Acid S-8®, and Orange oil.

Another embodiment comprises cyclosporin A, Ethofat 242/25®, and Orange oil.

Another embodiment comprises cyclosporin A, Hydropalat 65®, and Orange oil.

Another embodiment comprises cyclosporin A, Lipo EGMS®, and Orange oil.

Another embodiment comprises cyclosporin A, Lipopeg 2 DL®, and Orange oil.

Another embodiment comprises cyclosporin A, Lipopeg 4 DL®, and Orange oil.

Another embodiment comprises cyclosporin A, Lipopeg 4-L®, and Orange oil.

Another embodiment comprises cyclosporin A, Lipopeg 39-S®, and Orange oil.

Another embodiment comprises cyclosporin A, Lipopeg 4-S®, and Orange oil.

Another embodiment comprises cyclosporin A, Lipopeg 10-S®, and Orange oil.

Another embodiment comprises cyclosporin A, Lipopeg 100-S®, and Orange oil.

Another embodiment comprises cyclosporin A, Lipopeg 6000 DS®, and Orange oil.

Another embodiment comprises cyclosporin A, Lumulse 40-L®, and Orange oil.

Another embodiment comprises cyclosporin A, Lumulse 40-S®, and Orange oil.

Another embodiment comprises cyclosporin A, Lumulse 42-L®, and Orange oil.

Another embodiment comprises cyclosporin A, Lumulse 42-S®, and Orange oil.

Another embodiment comprises cyclosporin A, Lumulse 100-S®, and Orange oil.

Another embodiment comprises cyclosporin A, Lumulse 602-S®, and Orange oil.

Another embodiment comprises cyclosporin A, Magrabar PGE-20-0®, and Orange oil.

Another embodiment comprises cyclosporin A, Magrabar PGE-20L®, and Orange oil.

Another embodiment comprises cyclosporin A, Magrabar PGE-20T®, and Orange oil.

Another embodiment comprises cyclosporin A, Magrabar PGE-22-0®, and Orange oil.

Another embodiment comprises cyclosporin A, Magrabar PGE-22L®, and Orange oil.

Another embodiment comprises cyclosporin A, Magrabar PGE-22T®, and Orange oil.

Another embodiment comprises cyclosporin A, Magrabar PGE-40-0®, and Orange oil.

Another embodiment comprises cyclosporin A, Magrabar PGE-40L®, and Orange oil.

Another embodiment comprises cyclosporin A, Magrabar PGE-40T®, and Orange oil.

Another embodiment comprises cyclosporin A, Magrabar PGE-42-0®, and Orange oil.

Another embodiment comprises cyclosporin A, Magrabar PGE-42L®, and Orange oil.

Another embodiment comprises cyclosporin A, Magrabar PGE-42T®, and Orange oil.

Another embodiment comprises cyclosporin A, Magrabar PGE-60-0®, and Orange oil.

Another embodiment comprises cyclosporin A, Magrabar PGE-60L®, and Orange oil.

Another embodiment comprises cyclosporin A, Magrabar PGE-60T®, and Orange oil.

Another embodiment comprises cyclosporin A, Magrabar PGE-62-0®, and Orange oil.

Another embodiment comprises cyclosporin A, Magrabar PGE-62L®, and Orange oil.

Another embodiment comprises cyclosporin A, Magrabar PGE-62T®, and Orange oil.

Another embodiment comprises cyclosporin A, Mapeg S-40K®, and Orange oil.

Another embodiment comprises cyclosporin A, Marlowet OTS®, and Orange oil.

Another embodiment comprises cyclosporin A, Naturechem PGR®, and Orange oil.

Another embodiment comprises cyclosporin A, PG No. 4®, and Orange oil.

Another embodiment comprises cyclosporin A, Renex 20®, and Orange oil.

Another embodiment comprises cyclosporin A, Ritox 52®, and Orange oil.

Another embodiment comprises cyclosporin A, Ritox 53®, and Orange oil.

Another embodiment comprises cyclosporin A, Ritox 59®, and Orange oil.

Another embodiment comprises cyclosporin A, Surfax 8916/A®, and Orange oil.

Another embodiment comprises cyclosporin A, Tego Acid S 40 P®, and Orange oil.

Another embodiment comprises cyclosporin A, Tego Acid S 100 P®, and Orange oil.

Another embodiment comprises cyclosporin A, Tween 20®, and Orange oil.

Another embodiment comprises cyclosporin A, Volpo 131®, and Orange oil.

Another embodiment comprises cyclosporin A, an Ethoxylated Fatty Ester or Oil, and Orange oil.

Another embodiment comprises cyclosporin A, Acconon 6-C10®, and Orange oil.

Another embodiment comprises cyclosporin A, Acconon CC-6®, and Orange oil.

Another embodiment comprises cyclosporin A, Acconon CO-7®, and Orange oil.

Another embodiment comprises cyclosporin A, Aldosperse 40/60 FG®, and Orange oil.

Another embodiment comprises cyclosporin A, Aldosperse ML-23®, and Orange oil.

Another embodiment comprises cyclosporin A, Aldosperse MS-20 FG®, and Orange oil.

Another embodiment comprises cyclosporin A, Alkamuls EL-620®, and Orange oil.

Another embodiment comprises cyclosporin A, Alkamuls EL-719®, and Orange oil.

Another embodiment comprises cyclosporin A, Alkamuls EL-985®, and Orange oil.

Another embodiment comprises cyclosporin A, Arlatone G®, and Orange oil.

Another embodiment comprises cyclosporin A, Arlatone T®, and Orange oil.

Another embodiment comprises cyclosporin A, Atlas G-1045A®, and Orange oil.

Another embodiment comprises cyclosporin A, Atlas G-1086®, and Orange oil.

Another embodiment comprises cyclosporin A, Atlas G-1087®, and Orange oil.

Another embodiment comprises cyclosporin A, Atlas G-1089®, and Orange oil.

Another embodiment comprises cyclosporin A, Atlas G-1096®, and Orange oil.

Another embodiment comprises cyclosporin A, Atlas G-1292®, and Orange oil.

Another embodiment comprises cyclosporin A, Atlas G-1293®, and Orange oil.

Another embodiment comprises cyclosporin A, Atlas G-1300®, and Orange oil.

Another embodiment comprises cyclosporin A, Atlas G-7076®, and Orange oil.

Another embodiment comprises cyclosporin A, Capmul EMG®, and Orange oil.

Another embodiment comprises cyclosporin A, Chemonic CO-40®, and Orange oil.

Another embodiment comprises cyclosporin A, Chemonic LI-3®, and Orange oil.

Another embodiment comprises cyclosporin A, Chemonic LI-7®, and Orange oil.

Another embodiment comprises cyclosporin A, Cirrasol GM®, and Orange oil.

Another embodiment comprises cyclosporin A, Cremophor CO 40®, and Orange oil.

Another embodiment comprises cyclosporin A, Cremophor CO 410®, and Orange oil.

Another embodiment comprises cyclosporin A, Cremophor EL®, and Orange oil.

Another embodiment comprises cyclosporin A, Cremophor GC7®, and Orange oil.

Another embodiment comprises cyclosporin A, Cremophor RH-40®, and Orange oil.

Another embodiment comprises cyclosporin A, Crovol A-40®, and Orange oil.

Another embodiment comprises cyclosporin A, Crovol A-70®, and Orange oil.

Another embodiment comprises cyclosporin A, Crovol M-70®, and Orange oil.

Another embodiment comprises cyclosporin A, Crovol PK-70®, and Orange oil.

Another embodiment comprises cyclosporin A, Cutina E-24®, and Orange oil.

Another embodiment comprises cyclosporin A, Dacospin 12-P®, and Orange oil.

Another embodiment comprises cyclosporin A, Dehymuls HRE-7®, and Orange oil.

Another embodiment comprises cyclosporin A, DeSonic 30C®, and Orange oil.

Another embodiment comprises cyclosporin A, DeSonic 36C®, and Orange oil.

Another embodiment comprises cyclosporin A, DeSonic 40C®, and Orange oil.

Another embodiment comprises cyclosporin A, Durfax 60®, and Orange oil.

Another embodiment comprises cyclosporin A, Durfax 65®, and Orange oil.

Another embodiment comprises cyclosporin A, Durfax 80®, and Orange oil.

Another embodiment comprises cyclosporin A, Durfax EOM®, and Orange oil.

Another embodiment comprises cyclosporin A, Eccoterge NF-2®, and Orange oil.

Another embodiment comprises cyclosporin A, Emulpon CO-360®, and Orange oil.

Another embodiment comprises cyclosporin A, Emulpon CO-550®, and Orange oil.

Another embodiment comprises cyclosporin A, Emulsogen EL®, and Orange oil.

Another embodiment comprises cyclosporin A, Emulsogen HCO 040®, and Orange oil.

Another embodiment comprises cyclosporin A, Emulsogen HCO 060®, and Orange oil.

Another embodiment comprises cyclosporin A, Emulsynt 1055®, and Orange oil.

Another embodiment comprises cyclosporin A, Ethox 3095®, and Orange oil.

Another embodiment comprises cyclosporin A, Eumulgin RO-40®, and Orange oil.

Another embodiment comprises cyclosporin A, Genapol G-260®, and Orange oil.

Another embodiment comprises cyclosporin A, Glycosperse L-20®, and Orange oil.

Another embodiment comprises cyclosporin A, Glycosperse O-5®, and Orange oil.

Another embodiment comprises cyclosporin A, Glycosperse O-20®, and Orange oil.

Another embodiment comprises cyclosporin A, Glycosperse O-20 FG®, and Orange oil.

Another embodiment comprises cyclosporin A, Glycosperse S-20®, and Orange oil.

Another embodiment comprises cyclosporin A, Glycosperse S-20 FG®, and Orange oil.

Another embodiment comprises cyclosporin A, Glycosperse TS-20®, and Orange oil.

Another embodiment comprises cyclosporin A, Glycosperse TS-20 FG®, and Orange oil.

Another embodiment comprises cyclosporin A, Hetan SL®, and Orange oil.

Another embodiment comprises cyclosporin A, Hetan SO®, and Orange oil.

Another embodiment comprises cyclosporin A, Hetan SS®, and Orange oil.

Another embodiment comprises cyclosporin A, Hetoxide C-2®, and Orange oil.

Another embodiment comprises cyclosporin A, Hetoxide C-9®, and Orange oil.

Another embodiment comprises cyclosporin A, Hetoxide C-15®, and Orange oil.

Another embodiment comprises cyclosporin A, Hetoxide C-25®, and Orange oil.

Another embodiment comprises cyclosporin A, Hetoxide C-40®, and Orange oil.

Another embodiment comprises cyclosporin A, Hetoxide C-200®, and Orange oil.

Another embodiment comprises cyclosporin A, Hetoxide C-200-50%®, and Orange oil.

Another embodiment comprises cyclosporin A, Hetoxide GC-30®, and Orange oil.

Another embodiment comprises cyclosporin A, Hetoxide HC-60®, and Orange oil.

Another embodiment comprises cyclosporin A, Ice No. 2®, and Orange oil.

Another embodiment comprises cyclosporin A, Incrocas 30/40®, and Orange oil.

Another embodiment comprises cyclosporin A, Lexol EC®, and Orange oil.

Another embodiment comprises cyclosporin A, Lexol EO®, and Orange oil.

Another embodiment comprises cyclosporin A, Lipocol HCO-40®, and Orange oil.

Another embodiment comprises cyclosporin A, Lipocol HCO-60®, and Orange oil.

Another embodiment comprises cyclosporin A, Lipocol O-3 Special®, and Orange oil.

Another embodiment comprises cyclosporin A, Lipopeg 2-L®, and Orange oil.

Another embodiment comprises cyclosporin A, Lipopeg 4-DO®, and Orange oil.

Another embodiment comprises cyclosporin A, Lipopeg 4-DS®, and Orange oil.

Another embodiment comprises cyclosporin A, Lipovol GTB®, and Orange oil.

Another embodiment comprises cyclosporin A, Lonzest SML-20®, and Orange oil.

Another embodiment comprises cyclosporin A, Lonzest SMO-20®, and Orange oil.

Another embodiment comprises cyclosporin A, Lonzest SMS-20®, and Orange oil.

Another embodiment comprises cyclosporin A, Lonzest STO-20®, and Orange oil.

Another embodiment comprises cyclosporin A, Lonzest STS-20®, and Orange oil.

Another embodiment comprises cyclosporin A, Lumulse GR-40®, and Orange oil.

Another embodiment comprises cyclosporin A, Lumulse GRH-40®, and Orange oil.

Another embodiment comprises cyclosporin A, Lumulse POE (7) GML®, and Orange oil.

Another embodiment comprises cyclosporin A, Lumulse POE (12) Glyc®, and Orange oil.

Another embodiment comprises cyclosporin A, Lumulse POE (40) MS KP®, and Orange oil.

Another embodiment comprises cyclosporin A, Marlowet 4750®, and Orange oil.

Another embodiment comprises cyclosporin A, Marlowet LVS®, and Orange oil.

Another embodiment comprises cyclosporin A, Marlowet R 11®, and Orange oil.

Another embodiment comprises cyclosporin A, Marlowet R 40®, and Orange oil.

Another embodiment comprises cyclosporin A, Mazol 80 MGK®, and Orange oil.

Another embodiment comprises cyclosporin A, Nonionic Emulsifier T-9®, and Orange oil.

Another embodiment comprises cyclosporin A, Oronal LCG®, and Orange oil.

Another embodiment comprises cyclosporin A, Polyderm PPI-CO-200®, and Orange oil.

Another embodiment comprises cyclosporin A, Polyderm PPI-CO-40®, and Orange oil.

Another embodiment comprises cyclosporin A, Rewoderm LI 520-70®, and Orange oil.

Another embodiment comprises cyclosporin A, Ritapeg 150 DS®, and Orange oil.

Another embodiment comprises cyclosporin A, Softigen 767®, and Orange oil.

Another embodiment comprises cyclosporin A, Surfactol 318®, and Orange oil.

Another embodiment comprises cyclosporin A, Surfactol 365®, and Orange oil.

Another embodiment comprises cyclosporin A, Syn Lube 107®, and Orange oil.

Another embodiment comprises cyclosporin A, Syn Lube 728®, and Orange oil.

Another embodiment comprises cyclosporin A, Syn Lube 1632H®, and Orange oil.

Another embodiment comprises cyclosporin A, Syn Lube 6277-A®, and Orange oil.

Another embodiment comprises cyclosporin A, T-Det C-20®, and Orange oil.

Another embodiment comprises cyclosporin A, T-Det C-40®, and Orange oil.

Another embodiment comprises cyclosporin A, Tally 100 Plus®, and Orange oil.

Another embodiment comprises cyclosporin A, Uniperol EL®, and Orange oil.

Another embodiment comprises cyclosporin A, a Fatty Ester, and Orange oil.

Another embodiment comprises cyclosporin A, Actralube-Syn 147®, and Orange oil.

Another embodiment comprises cyclosporin A, Atlas G-1556®, and Orange oil.

Another embodiment comprises cyclosporin A, Atlas G-1564®, and Orange oil.

Another embodiment comprises cyclosporin A, Atlasol Base Oil S®, and Orange oil.

Another embodiment comprises cyclosporin A, Base ML®, and Orange oil.

Another embodiment comprises cyclosporin A, Base MT®, and Orange oil.

Another embodiment comprises cyclosporin A, Cerasynt 303®, and Orange oil.

Another embodiment comprises cyclosporin A, Dermol 1012®, and Orange oil.

Another embodiment comprises cyclosporin A, Kemester 4000®, and Orange oil.

Another embodiment comprises cyclosporin A, Lactipol S®, and Orange oil.

Another embodiment comprises cyclosporin A, Magrabar PGO®, and Orange oil.

Another embodiment comprises cyclosporin A, Mayco Base BFO®, and Orange oil.

Another embodiment comprises cyclosporin A, Methyl Linoleate®, and Orange oil.

Another embodiment comprises cyclosporin A, Pationic 122A®, and Orange oil.

Another embodiment comprises cyclosporin A, Pationic 138C®, and Orange oil.

Another embodiment comprises cyclosporin A, Pationic CSL®, and Orange oil.

Another embodiment comprises cyclosporin A, Pationic ISL®, and Orange oil.

Another embodiment comprises cyclosporin A, Pationic SBL®, and Orange oil.

Another embodiment comprises cyclosporin A, Pationic SSL®, and Orange oil.

Another embodiment comprises cyclosporin A, Ritasol®, and Orange oil.

Another embodiment comprises cyclosporin A, Tego Alkanol CS 20®, and Orange oil.

Another embodiment comprises cyclosporin A, Tego Alkanol L23 P®, and Orange oil.

Another embodiment comprises cyclosporin A, Tego Alkanol S2®, and Orange oil.

Another embodiment comprises cyclosporin A, Tego Alkanol S20 P®, and Orange oil.

Another embodiment comprises cyclosporin A, Triemulsifier 600 MS®, and Orange oil.

Another embodiment comprises cyclosporin A, a Glycerol Esters, and Orange oil.

Another embodiment comprises cyclosporin A, Agro #9 Wint SBO®, and Orange oil.

Another embodiment comprises cyclosporin A, Ahcovel Base 700®, and Orange oil.

Another embodiment comprises cyclosporin A, Aldo HMS FG®, and Orange oil.

Another embodiment comprises cyclosporin A, Aldo MLD®, and Orange oil.

Another embodiment comprises cyclosporin A, Aldo MLD FG®, and Orange oil.

Another embodiment comprises cyclosporin A, Aldo MO FG®, and Orange oil.

Another embodiment comprises cyclosporin A, Aldo MS®, and Orange oil.

Another embodiment comprises cyclosporin A, Aldo MS FG®, and Orange oil.

Another embodiment comprises cyclosporin A, Aldo MS LG FG®, and Orange oil.

Another embodiment comprises cyclosporin A, Aldo MSD®, and Orange oil.

Another embodiment comprises cyclosporin A, Aldo MSD FG®, and Orange oil.

Another embodiment comprises cyclosporin A, Aldosperse O-20 FG®, and Orange oil.

Another embodiment comprises cyclosporin A, Aldosperse TS-20 FG®, and Orange oil.

Another embodiment comprises cyclosporin A, Aldosperse TS-40 FG®, and Orange oil.

Another embodiment comprises cyclosporin A, Arlacel 165®, and Orange oil.

Another embodiment comprises cyclosporin A, Arlacel 186®, and Orange oil.

Another embodiment comprises cyclosporin A, Capmul GMO®, and Orange oil.

Another embodiment comprises cyclosporin A, Capmul GMS®, and Orange oil.

Another embodiment comprises cyclosporin A, Caprol 3GO®, and Orange oil.

Another embodiment comprises cyclosporin A, Caprol 3GVS®, and Orange oil.

Another embodiment comprises cyclosporin A, Caprol 6G2S®, and Orange oil.

Another embodiment comprises cyclosporin A, Caprol 10G40®, and Orange oil.

Another embodiment comprises cyclosporin A, Caprol 10G100®, and Orange oil.

Another embodiment comprises cyclosporin A, Caprol ET®, and Orange oil.

Another embodiment comprises cyclosporin A, Caprol PGE860®, and Orange oil.

Another embodiment comprises cyclosporin A, Cerasynt 945®, and Orange oil.

Another embodiment comprises cyclosporin A, Cerasynt GMS®, and Orange oil.

Another embodiment comprises cyclosporin A, Cerasynt Q®, and Orange oil.

Another embodiment comprises cyclosporin A, Cerasynt SD®, and Orange oil.

Another embodiment comprises cyclosporin A, Cerasynt WM®, and Orange oil.

Another embodiment comprises cyclosporin A, Chemsperse 14®, and Orange oil.

Another embodiment comprises cyclosporin A, Cremophor GO-32®, and Orange oil.

Another embodiment comprises cyclosporin A, Cremophor GS11®, and Orange oil.

Another embodiment comprises cyclosporin A, Cremophor GS-32®, and Orange oil.

Another embodiment comprises cyclosporin A, Cutina KD-16®, and Orange oil.

Another embodiment comprises cyclosporin A, Dehymuls PGPH®, and Orange oil.

Another embodiment comprises cyclosporin A, Dermol DGDIS®, and Orange oil.

Another embodiment comprises cyclosporin A, Dermol DGMIS®, and Orange oil.

Another embodiment comprises cyclosporin A, Dermol G-76®, and Orange oil.

Another embodiment comprises cyclosporin A, Dermol G-7DI®, and Orange oil.

Another embodiment comprises cyclosporin A, Dermol NGDI®, and Orange oil.

Another embodiment comprises cyclosporin A, Dermolan GLH®, and Orange oil.

Another embodiment comprises cyclosporin A, Drewmulse GMO®, and Orange oil.

Another embodiment comprises cyclosporin A, Drewpol 3-5-M®, and Orange oil.

Another embodiment comprises cyclosporin A, Durlac 100 W®, and Orange oil.

Another embodiment comprises cyclosporin A, Dur-Lo®, and Orange oil.

Another embodiment comprises cyclosporin A, Dynasan 118®, and Orange oil.

Another embodiment comprises cyclosporin A, EC-25®, and Orange oil.

Another embodiment comprises cyclosporin A, EM 40®, and Orange oil.

Another embodiment comprises cyclosporin A, Emerest 2400®, and Orange oil.

Another embodiment comprises cyclosporin A, Emerest 2452®, and Orange oil.

Another embodiment comprises cyclosporin A, Empilan G-26®, and Orange oil.

Another embodiment comprises cyclosporin A, Genapol TSM®, and Orange oil.

Another embodiment comprises cyclosporin A, Hostacerin DGI®, and Orange oil.

Another embodiment comprises cyclosporin A, Hostacerin DGL®, and Orange oil.

Another embodiment comprises cyclosporin A, Hostacerin DGMS®, and Orange oil.

Another embodiment comprises cyclosporin A, Hostacerin DGSB®, and Orange oil.

Another embodiment comprises cyclosporin A, Ice No. 2®, and Orange oil.

Another embodiment comprises cyclosporin A, Imwitor 742®, and Orange oil.

Another embodiment comprises cyclosporin A, Imwitor 780 K®, and Orange oil.

Another embodiment comprises cyclosporin A, Imwitor 960 Flakes®, and Orange oil.

Another embodiment comprises cyclosporin A, Isolan GI 34®, and Orange oil.

Another embodiment comprises cyclosporin A, Isolan GO 33®, and Orange oil.

Another embodiment comprises cyclosporin A, Kemester 1000®, and Orange oil.

Another embodiment comprises cyclosporin A, Kemester 2000®, and Orange oil.

Another embodiment comprises cyclosporin A, Kemester 2000®, and Orange oil.

Another embodiment comprises cyclosporin A, Kemester 6000SE®, and Orange oil.

Another embodiment comprises cyclosporin A, Lamecreme DGE 18®, and Orange oil.

Another embodiment comprises cyclosporin A, Lexemul 515®, and Orange oil.

Another embodiment comprises cyclosporin A, Lexemul 561®, and Orange oil.

Another embodiment comprises cyclosporin A, Lexemul AR®, and Orange oil.

Another embodiment comprises cyclosporin A, Lexemul AS®, and Orange oil.

Another embodiment comprises cyclosporin A, Lexemul GDL®, and Orange oil.

Another embodiment comprises cyclosporin A, Lexemul T®, and Orange oil.

Another embodiment comprises cyclosporin A, Lipomulse 165®, and Orange oil.

Another embodiment comprises cyclosporin A, Lumulse GML K®, and Orange oil.

Another embodiment comprises cyclosporin A, Lumulse GMO K®, and Orange oil.

Another embodiment comprises cyclosporin A, Lumulse GMR K®, and Orange oil.

Another embodiment comprises cyclosporin A, Lumulse GMT K®, and Orange oil.

Another embodiment comprises cyclosporin A, Magrabar GMC®, and Orange oil.

Another embodiment comprises cyclosporin A, Magrabar GMO-CK®, and Orange oil.

Another embodiment comprises cyclosporin A, Magrabar MDG-5050®, and Orange oil.

Another embodiment comprises cyclosporin A, Magrabar PGO-315®, and Orange oil.

Another embodiment comprises cyclosporin A, Magrabar PGO-1010®, and Orange oil.

Another embodiment comprises cyclosporin A, Mazol 300K®, and Orange oil.

Another embodiment comprises cyclosporin A, Mazol GMO-K®, and Orange oil.

Another embodiment comprises cyclosporin A, Mazol GMS-K®, and Orange oil.

Another embodiment comprises cyclosporin A, Mazol PG031-K®, and Orange oil.

Another embodiment comprises cyclosporin A, Miglyol 812®, and Orange oil.

Another embodiment comprises cyclosporin A, Norfox 165C®, and Orange oil.

Another embodiment comprises cyclosporin A, Schercemol GMIS®, and Orange oil.

Another embodiment comprises cyclosporin A, Tegin®, and Orange oil.

Another embodiment comprises cyclosporin A, Tegin 4100 Pellets®, and Orange oil.

Another embodiment comprises cyclosporin A, Tegin M Pellets®, and Orange oil.

Another embodiment comprises cyclosporin A, Tegin OV®, and Orange oil.

Another embodiment comprises cyclosporin A, Teginacid H®, and Orange oil.

Another embodiment comprises cyclosporin A, Tego Cosmo P813®, and Orange oil.

Another embodiment comprises cyclosporin A, Wickenol 535®, and Orange oil.

Another embodiment comprises cyclosporin A, Witconol 14®, and Orange oil.

Another embodiment comprises cyclosporin A, Witconol 1 4®, and Orange oil.

Another embodiment comprises cyclosporin A, Witconol 14F®, and Orange oil.

Another embodiment comprises cyclosporin A, Witconol 18L®, and Orange oil.

Another embodiment comprises cyclosporin A, Witconol GOT®, and Orange oil.

Another embodiment comprises cyclosporin A, Witconol MST®, and Orange oil.

Another embodiment comprises cyclosporin A, Witconol RHT®, and Orange oil.

Another embodiment comprises cyclosporin A, a Glycol Ester, and Orange oil.

Another embodiment comprises cyclosporin A, Alkamuls 600 DO®, and Orange oil.

Another embodiment comprises cyclosporin A, Alkamuls SEG®, and Orange oil.

Another embodiment comprises cyclosporin A, Atlas EM-2®, and Orange oil.

Another embodiment comprises cyclosporin A, Cerasynt IP®, and Orange oil.

Another embodiment comprises cyclosporin A, Cerasynt M®, and Orange oil.

Another embodiment comprises cyclosporin A, Cerasynt MN®, and Orange oil.

Another embodiment comprises cyclosporin A, Cerasynt PA®, and Orange oil.

Another embodiment comprises cyclosporin A, Chemsperse EGDS®, and Orange oil.

Another embodiment comprises cyclosporin A, Chemsperse EGMS®, and Orange oil.

Another embodiment comprises cyclosporin A, Colonial Monolaurin®, and Orange oil.

Another embodiment comprises cyclosporin A, DeMuls SGE-95®, and Orange oil.

Another embodiment comprises cyclosporin A, Eccoterge 200®, and Orange oil.

Another embodiment comprises cyclosporin A, Emerest 2380®, and Orange oil.

Another embodiment comprises cyclosporin A, Ethox 2610®, and Orange oil.

Another embodiment comprises cyclosporin A, Ethox DO-9®, and Orange oil.

Another embodiment comprises cyclosporin A, Ethox DO-14®, and Orange oil.

Another embodiment comprises cyclosporin A, Ethox SO-9®, and Orange oil.

Another embodiment comprises cyclosporin A, Fizul MD-318®, and Orange oil.

Another embodiment comprises cyclosporin A, Genapol EGDS-VHP®, and Orange oil.

Another embodiment comprises cyclosporin A, Genapol TS Powder®, and Orange oil.

Another embodiment comprises cyclosporin A, Hostacerin WO®, and Orange oil.

Another embodiment comprises cyclosporin A, Inversol 140®, and Orange oil.

Another embodiment comprises cyclosporin A, Kemester 104®, and Orange oil.

Another embodiment comprises cyclosporin A, Kemester 205®, and Orange oil.

Another embodiment comprises cyclosporin A, Kemester 226®, and Orange oil.

Another embodiment comprises cyclosporin A, Kemester 5221 SE®, and Orange oil.

Another embodiment comprises cyclosporin A, Kemester EGDS®, and Orange oil.

Another embodiment comprises cyclosporin A, Lexemul EGDS®, and Orange oil.

Another embodiment comprises cyclosporin A, Lexemul EGMS®, and Orange oil.

Another embodiment comprises cyclosporin A, Lexemul P®, and Orange oil.

Another embodiment comprises cyclosporin A, Lipo DGLS®, and Orange oil.

Another embodiment comprises cyclosporin A, Lipo EGDS®, and Orange oil.

Another embodiment comprises cyclosporin A, Lipo PGMS®, and Orange oil.

Another embodiment comprises cyclosporin A, Liposorb S-4®, and Orange oil.

Another embodiment comprises cyclosporin A, Liposorb TO-20®, and Orange oil.

Another embodiment comprises cyclosporin A, Lumulse PGO®, and Orange oil.

Another embodiment comprises cyclosporin A, Mackester EGDS®, and Orange oil.

Another embodiment comprises cyclosporin A, Mackester EGMS®, and Orange oil.

Another embodiment comprises cyclosporin A, Mackester GSTP®, and Orange oil.

Another embodiment comprises cyclosporin A, Mackester Series®, and Orange oil.

Another embodiment comprises cyclosporin A, Magrabar PDG-50®, and Orange oil.

Another embodiment comprises cyclosporin A, Mapeg 6000 DS®, and Orange oil.

Another embodiment comprises cyclosporin A, Marlowet 4702®, and Orange oil.

Another embodiment comprises cyclosporin A, Monalube 305®, and Orange oil.

Another embodiment comprises cyclosporin A, Monalube 310®, and Orange oil.

Another embodiment comprises cyclosporin A, Monalube 315®, and Orange oil.

Another embodiment comprises cyclosporin A, Monalube 320®, and Orange oil.

Another embodiment comprises cyclosporin A, Monalube 325®, and Orange oil.

Another embodiment comprises cyclosporin A, Monalube 330®, and Orange oil.

Another embodiment comprises cyclosporin A, Naturechem PGHS®, and Orange oil.

Another embodiment comprises cyclosporin A, Polycastorol PLO-840®, and Orange oil.

Another embodiment comprises cyclosporin A, Polytex 10M®, and Orange oil.

Another embodiment comprises cyclosporin A, Ritasynt IP®, and Orange oil.

Another embodiment comprises cyclosporin A, Ross Chem PEG 600 DT®, and Orange oil.

Another embodiment comprises cyclosporin A, Schercemol PGMS®, and Orange oil.

Another embodiment comprises cyclosporin A, Sponto H-44C®, and Orange oil.

Another embodiment comprises cyclosporin A, Tegin G®, and Orange oil.

Another embodiment comprises cyclosporin A, Witbreak DGE-182®, and Orange oil.

Another embodiment comprises cyclosporin A, Witbreak DGE-182®, and Orange oil.

Another embodiment comprises cyclosporin A, Witbreak DRA-21®, and Orange oil.

Another embodiment comprises cyclosporin A, Witbreak DRA-21®, and Orange oil.

Another embodiment comprises cyclosporin A, Witbreak DRA-50®, and Orange oil.

Another embodiment comprises cyclosporin A, Witbreak DRA-50®, and Orange oil.

Another embodiment comprises cyclosporin A, Witconol F46-46®, and Orange oil.

Another embodiment comprises cyclosporin A, Witconol H-32®, and Orange oil.

Another embodiment comprises cyclosporin A, Witconol H-33®, and Orange oil.

Another embodiment comprises cyclosporin A, Witconol H-35A®, and Orange oil.

Another embodiment comprises cyclosporin A, Witconol RHP®, and Orange oil.

Another embodiment comprises cyclosporin A, a Lanolin-based Derivative, and Orange oil.

Another embodiment comprises cyclosporin A, Amerchol CAB®, and Orange oil.

Another embodiment comprises cyclosporin A, Amerchol L-101®, and Orange oil.

Another embodiment comprises cyclosporin A, Amerlate LFA-LO®, and Orange oil.

Another embodiment comprises cyclosporin A, Amerlate P®, and Orange oil.

Another embodiment comprises cyclosporin A, Barre Common Degras®, and Orange oil.

Another embodiment comprises cyclosporin A, Cholesterol NF®, and Orange oil.

Another embodiment comprises cyclosporin A, Crodalan AWS®, and Orange oil.

Another embodiment comprises cyclosporin A, Crodalan LA®, and Orange oil.

Another embodiment comprises cyclosporin A, Emery 1650®, and Orange oil.

Another embodiment comprises cyclosporin A, Emery 1650®, and Orange oil.

Another embodiment comprises cyclosporin A, Emery 1740®, and Orange oil.

Another embodiment comprises cyclosporin A, Emery 1740®, and Orange oil.

Another embodiment comprises cyclosporin A, Forlan 500®, and Orange oil.

Another embodiment comprises cyclosporin A, Forlan L®, and Orange oil.

Another embodiment comprises cyclosporin A, Laneto 50®, and Orange oil.

Another embodiment comprises cyclosporin A, Laneto 100®, and Orange oil.

Another embodiment comprises cyclosporin A, Laneto AWS®, and Orange oil.

Another embodiment comprises cyclosporin A, Lanfrax 1776®, and Orange oil.

Another embodiment comprises cyclosporin A, Lanfrax 1776®, and Orange oil.

Another embodiment comprises cyclosporin A, Lanogel 21®, and Orange oil.

Another embodiment comprises cyclosporin A, Lipolan®, and Orange oil.

Another embodiment comprises cyclosporin A, Lipolan 31®, and Orange oil.

Another embodiment comprises cyclosporin A, OHlan®, and Orange oil.

Another embodiment comprises cyclosporin A, Polychol 5®, and Orange oil.

Another embodiment comprises cyclosporin A, Polychol 15®, and Orange oil.

Another embodiment comprises cyclosporin A, Ritacetyl®, and Orange oil.

Another embodiment comprises cyclosporin A, Ritachol®, and Orange oil.

Another embodiment comprises cyclosporin A, Ritahydrox®, and Orange oil.

Another embodiment comprises cyclosporin A, Ritalafa®, and Orange oil.

Another embodiment comprises cyclosporin A, Ritalan®, and Orange oil.

Another embodiment comprises cyclosporin A, Ritalan AWS®, and Orange oil.

Another embodiment comprises cyclosporin A, Ritalan C®, and Orange oil.

Another embodiment comprises cyclosporin A, Ritawax®, and Orange oil.

Another embodiment comprises cyclosporin A, Ritawax AEO®, and Orange oil.

Another embodiment comprises cyclosporin A, Ritawax ALA®, and Orange oil.

Another embodiment comprises cyclosporin A, Solan/Solan 50/Super Solan®, and Orange oil.

Another embodiment comprises cyclosporin A, Super Hartolan/Hartolan®, and Orange oil.

Another embodiment comprises cyclosporin A, Supersat AWS-4®, and Orange oil.

Another embodiment comprises cyclosporin A, Supersat AWS-24®, and Orange oil.

Another embodiment comprises cyclosporin A, a Lecithin or Lecithin Derivative, and Orange oil.

Another embodiment comprises cyclosporin A, Alcolec®, and Orange oil.

Another embodiment comprises cyclosporin A, Lecithin®, and Orange oil.

Another embodiment comprises cyclosporin A, Lexin K®, and Orange oil.

Another embodiment comprises cyclosporin A, Natipide®, and Orange oil.

Another embodiment comprises cyclosporin A, a Lignin or Lignin Derivative, and Orange oil.

Another embodiment comprises cyclosporin A, Diwatex XP 9®, and Orange oil.

Another embodiment comprises cyclosporin A, Dynasperse LCD®, and Orange oil.

Another embodiment comprises cyclosporin A, Indulin SAL®, and Orange oil.

Another embodiment comprises cyclosporin A, Indulin W-1®, and Orange oil.

Another embodiment comprises cyclosporin A, Indulin W-5®, and Orange oil.

Another embodiment comprises cyclosporin A, Lignosol FTA®, and Orange oil.

Another embodiment comprises cyclosporin A, Lignosol SFX-65®, and Orange oil.

Another embodiment comprises cyclosporin A, Marasperse 52 CP®, and Orange oil.

Another embodiment comprises cyclosporin A, Marasperse AG®, and Orange oil.

Another embodiment comprises cyclosporin A, Marasperse CBOS-4®, and Orange oil.

Another embodiment comprises cyclosporin A, Ufoxane 2®, and Orange oil.

Another embodiment comprises cyclosporin A, a Methyl Ester, and Orange oil.

Another embodiment comprises cyclosporin A, E.B. Cleaner AK®, and Orange oil.

Another embodiment comprises cyclosporin A, Oleocal ME-70®, and Orange oil.

Another embodiment comprises cyclosporin A, Trade Name Company®, and Orange oil.

Another embodiment comprises cyclosporin A, Methyl Esters (cont'd)®, and Orange oil.

Another embodiment comprises cyclosporin A, Oleocal ME-92®, and Orange oil.

Another embodiment comprises cyclosporin A, Oleocal ME-112®, and Orange oil.

Another embodiment comprises cyclosporin A, Oleocal ME-130®, and Orange oil.

Another embodiment comprises cyclosporin A, a Monoglyceride or a Derivative thereof, and Orange oil.

Another embodiment comprises cyclosporin A, Dynacet 211®, and Orange oil.

Another embodiment comprises cyclosporin A, Hetsorb S-20®, and Orange oil.

Another embodiment comprises cyclosporin A, Imwitor 191®, and Orange oil.

Another embodiment comprises cyclosporin A, Imwitor 370®, and Orange oil.

Another embodiment comprises cyclosporin A, Imwitor 375®, and Orange oil.

Another embodiment comprises cyclosporin A, Imwitor 900®, and Orange oil.

Another embodiment comprises cyclosporin A, Imwitor 945®, and Orange oil.

Another embodiment comprises cyclosporin A, Imwitor 2020®, and Orange oil.

Another embodiment comprises cyclosporin A, Kemester 5500®, and Orange oil.

Another embodiment comprises cyclosporin A, Kemester 6000®, and Orange oil.

Another embodiment comprises cyclosporin A, Magrabar GMC®, and Orange oil.

Another embodiment comprises cyclosporin A, Magrabar GMO-CK®, and Orange oil.

Another embodiment comprises cyclosporin A, Magrabar GPC-10®, and Orange oil.

Another embodiment comprises cyclosporin A, Magrabar MDG-5050®, and Orange oil.

Another embodiment comprises cyclosporin A, Monalube 335®, and Orange oil.

Another embodiment comprises cyclosporin A, Monoglycerides Glyceryl Monestearate Archer Daniels Midland Company®, and Orange oil.

Another embodiment comprises cyclosporin A, Rita GMS®, and Orange oil.

Another embodiment comprises cyclosporin A, Ritamulse SCG®, and Orange oil.

Another embodiment comprises cyclosporin A, Softigen 701®, and Orange oil.

Another embodiment comprises cyclosporin A, Tally 100 Plus®, and Orange oil.

Another embodiment comprises cyclosporin A, a Polyethylene Glycol, and Orange oil.

Another embodiment comprises cyclosporin A, Emulgade PL 68/50®, and Orange oil.

Another embodiment comprises cyclosporin A, Lumulse PEG®, and Orange oil.

Another embodiment comprises cyclosporin A, Rhodasurf PEG-400®, and Orange oil.

Another embodiment comprises cyclosporin A, Rhodasurf PEG-600®, and Orange oil.

Another embodiment comprises cyclosporin A, Witconol PEG-400®, and Orange oil.

Another embodiment comprises cyclosporin A, a Polymeric Surfactant, and Orange oil.

Another embodiment comprises cyclosporin A, Acritamer PNC-EG®, and Orange oil.

Another embodiment comprises cyclosporin A, Ag-Rho DEP-775®, and Orange oil.

Another embodiment comprises cyclosporin A, APG 325N Glycoside®, and Orange oil.

Another embodiment comprises cyclosporin A, Aristoflex AVC®, and Orange oil.

Another embodiment comprises cyclosporin A, Aristoflex HMB®, and Orange oil.

Another embodiment comprises cyclosporin A, Burco NPS-225®, and Orange oil.

Another embodiment comprises cyclosporin A, Burco NPS-816®, and Orange oil.

Another embodiment comprises cyclosporin A, Chemccinate 5603®, and Orange oil.

Another embodiment comprises cyclosporin A, Cosmedia Guar C-261N®, and Orange oil.

Another embodiment comprises cyclosporin A, Gantrez S-95®, and Orange oil.

Another embodiment comprises cyclosporin A, Glucopon 220 UP®, and Orange oil.

Another embodiment comprises cyclosporin A, Glucopon 225 DK®, and Orange oil.

Another embodiment comprises cyclosporin A, Glucopon 425 N®, and Orange oil.

Another embodiment comprises cyclosporin A, Glucopon 600 UP®, and Orange oil.

Another embodiment comprises cyclosporin A, Glucopon 625 UP®, and Orange oil.

Another embodiment comprises cyclosporin A, Pemulen 1621®, and Orange oil.

Another embodiment comprises cyclosporin A, Pemulen 1622®, and Orange oil.

Another embodiment comprises cyclosporin A, Pemulen TR-1®, and Orange oil.

Another embodiment comprises cyclosporin A, Pemulen TR-2®, and Orange oil.

Another embodiment comprises cyclosporin A, Plantacare 818®, and Orange oil.

Another embodiment comprises cyclosporin A, Plantapon LGC Sorb®, and Orange oil.

Another embodiment comprises cyclosporin A, Plantaren 1200N®, and Orange oil.

Another embodiment comprises cyclosporin A, Plantaren 2000N®, and Orange oil.

Another embodiment comprises cyclosporin A, Viscolam AT 64®, and Orange oil.

Another embodiment comprises cyclosporin A, Viscolam AT 64P®, and Orange oil.

Another embodiment comprises cyclosporin A, Viscolam AT 100®, and Orange oil.

Another embodiment comprises cyclosporin A, Viscolam MAC 7®, and Orange oil.

Another embodiment comprises cyclosporin A, Viscolam SMC 20®, and Orange oil.

Another embodiment comprises cyclosporin A, Witbreak RTC-323®, and Orange oil.

Another embodiment comprises cyclosporin A, WSI 3700®, and Orange oil.

Another embodiment comprises cyclosporin A, a Propoxylated or Ethoxylated Fatty Acid, Alcohol, or Alkyl Phenol, and Orange oil.

Another embodiment comprises cyclosporin A, Antarox AA-60®, and Orange oil.

Another embodiment comprises cyclosporin A, Antarox LF-224®, and Orange oil.

Another embodiment comprises cyclosporin A, Burcomul DFE-45®, and Orange oil.

Another embodiment comprises cyclosporin A, Burcoterge LFE-1000®, and Orange oil.

Another embodiment comprises cyclosporin A, Chemal LF-25B®, and Orange oil.

Another embodiment comprises cyclosporin A, Chemal LF-40B®, and Orange oil.

Another embodiment comprises cyclosporin A, Dehypon LS-36®, and Orange oil.

Another embodiment comprises cyclosporin A, Dehypon LS-36®, and Orange oil.

Another embodiment comprises cyclosporin A, Dehypon LS-54®, and Orange oil.

Another embodiment comprises cyclosporin A, Dehypon LS-54®, and Orange oil.

Another embodiment comprises cyclosporin A, Delonic 100 VLF®, and Orange oil.

Another embodiment comprises cyclosporin A, Delonic LF-60 MOD®, and Orange oil.

Another embodiment comprises cyclosporin A, Empiderm B®, and Orange oil.

Another embodiment comprises cyclosporin A, Ethylan 1206®, and Orange oil.

Another embodiment comprises cyclosporin A, Ethylan NS-500K®, and Orange oil.

Another embodiment comprises cyclosporin A, Ethylan NS-500LQ®, and Orange oil.

Another embodiment comprises cyclosporin A, Genapol 1392®, and Orange oil.

Another embodiment comprises cyclosporin A, Genapol 2317®, and Orange oil.

Another embodiment comprises cyclosporin A, Genapol 26EP710®, and Orange oil.

Another embodiment comprises cyclosporin A, Genapol EP 1022®, and Orange oil.

Another embodiment comprises cyclosporin A, Genapol EP 1024®, and Orange oil.

Another embodiment comprises cyclosporin A, Genapol EP 6068®, and Orange oil.

Another embodiment comprises cyclosporin A, Genapol NP915®, and Orange oil.

Another embodiment comprises cyclosporin A, Kieralon MFB®, and Orange oil.

Another embodiment comprises cyclosporin A, Lumisolve CSA-80 V®, and Orange oil.

Another embodiment comprises cyclosporin A, Marlowet 5001®, and Orange oil.

Another embodiment comprises cyclosporin A, Marlox FK 64®, and Orange oil.

Another embodiment comprises cyclosporin A, Marlox MO 124®, and Orange oil.

Another embodiment comprises cyclosporin A, Marlox S 58®, and Orange oil.

Another embodiment comprises cyclosporin A, Nonatell 1003®, and Orange oil.

Another embodiment comprises cyclosporin A, Nonatell 1038®, and Orange oil.

Another embodiment comprises cyclosporin A, Nonatell 1052®, and Orange oil.

Another embodiment comprises cyclosporin A, Nonatell 1061®, and Orange oil.

Another embodiment comprises cyclosporin A, Nonatell 1075®, and Orange oil.

Another embodiment comprises cyclosporin A, Nonatell 1088®, and Orange oil.

Another embodiment comprises cyclosporin A, Nonatell 1123®, and Orange oil.

Another embodiment comprises cyclosporin A, Nonatell 1153®, and Orange oil.

Another embodiment comprises cyclosporin A, Nonatell 1161®, and Orange oil.

Another embodiment comprises cyclosporin A, Nonatell 1172®, and Orange oil.

Another embodiment comprises cyclosporin A, Nonatell 1181®, and Orange oil.

Another embodiment comprises cyclosporin A, Norfox 36®, and Orange oil.

Another embodiment comprises cyclosporin A, Procetyl AWS®, and Orange oil.

Another embodiment comprises cyclosporin A, Sandoxylate SX 412®, and Orange oil.

Another embodiment comprises cyclosporin A, Sandoxylate SX 418®, and Orange oil.

Another embodiment comprises cyclosporin A, Surfonic JL-80X®, and Orange oil.

Another embodiment comprises cyclosporin A, Surfonic JL-80X-B1®, and Orange oil.

Another embodiment comprises cyclosporin A, Surfonic L4-29X®, and Orange oil.

Another embodiment comprises cyclosporin A, Surfonic LF®, and Orange oil.

Another embodiment comprises cyclosporin A, T-Det A826®, and Orange oil.

Another embodiment comprises cyclosporin A, T-Det LF-416®, and Orange oil.

Another embodiment comprises cyclosporin A, Tergitol Min-Foam 1X®, and Orange oil.

Another embodiment comprises cyclosporin A, Tergitol Min-Foam 2X®, and Orange oil.

Another embodiment comprises cyclosporin A, Triton CF-21®, and Orange oil.

Another embodiment comprises cyclosporin A, Triton CF-76®, and Orange oil.

Another embodiment comprises cyclosporin A, Triton XL-80N®, and Orange oil.

Another embodiment comprises cyclosporin A, Witconol NS-98®, and Orange oil.

Another embodiment comprises cyclosporin A, Witconol NS-108LQ®, and Orange oil.

Another embodiment comprises cyclosporin A, Witconol NS-145®, and Orange oil.

Another embodiment comprises cyclosporin A, Witconol NS-179®, and Orange oil.

Another embodiment comprises cyclosporin A, a Protein-based Surfactant, and Orange oil.

Another embodiment comprises cyclosporin A, AminoFoam W®, and Orange oil.

Another embodiment comprises cyclosporin A, Amiter LGOD-2®, and Orange oil.

Another embodiment comprises cyclosporin A, Amiter LGS-2®, and Orange oil.

Another embodiment comprises cyclosporin A, Amiter LGS-5®, and Orange oil.

Another embodiment comprises cyclosporin A, Lamepon S®, and Orange oil.

Another embodiment comprises cyclosporin A, Lamepon S®, and Orange oil.

Another embodiment comprises cyclosporin A, Maypon 4C®, and Orange oil.

Another embodiment comprises cyclosporin A, May-Tein C®, and Orange oil.

Another embodiment comprises cyclosporin A, May-Tein CT®, and Orange oil.

Another embodiment comprises cyclosporin A, May-Tein KTS®, and Orange oil.

Another embodiment comprises cyclosporin A, May-Tein SY®, and Orange oil.

Another embodiment comprises cyclosporin A, Plantapon S®, and Orange oil.

Another embodiment comprises cyclosporin A, Proteol APL®, and Orange oil.

Another embodiment comprises cyclosporin A, Proteol OAT®, and Orange oil.

Another embodiment comprises cyclosporin A, Pyroter CPI-40®, and Orange oil.

Another embodiment comprises cyclosporin A, Pyroter GPI-25®, and Orange oil.

Another embodiment comprises cyclosporin A, Supro-Tein S®, and Orange oil.

Another embodiment comprises cyclosporin A, Supro-Tein V®, and Orange oil.

Another embodiment comprises cyclosporin A,® and Orange oil.

Another embodiment comprises cyclosporin A, a Sarcosine Derivative®, and Orange oil.

Another embodiment comprises cyclosporin A, Crodasinic LS-30®, and Orange oil.

Another embodiment comprises cyclosporin A, Vanseal CS®, and Orange oil.

Another embodiment comprises cyclosporin A, Vanseal LS®, and Orange oil.

Another embodiment comprises cyclosporin A, Vanseal MS®, and Orange oil.

Another embodiment comprises cyclosporin A, Vanseal NACS-30®, and Orange oil.

Another embodiment comprises cyclosporin A, Vanseal NALS-95®, and Orange oil.

Another embodiment comprises cyclosporin A, Vanseal OS®, and Orange oil.

Another embodiment comprises cyclosporin A,® and Orange oil.

Another embodiment comprises cyclosporin A, a Silicone-based Sunactant®, and Orange oil.

Another embodiment comprises cyclosporin A, Abil-B-9950®, and Orange oil.

Another embodiment comprises cyclosporin A, Abil Care 85®, and Orange oil.

Another embodiment comprises cyclosporin A, Abil EM 90g®, and Orange oil.

Another embodiment comprises cyclosporin A, Abil EM 97®, and Orange oil.

Another embodiment comprises cyclosporin A, Abil WE-09®, and Orange oil.

Another embodiment comprises cyclosporin A, Dow Corning 1248 Fluid®, and Orange oil.

Another embodiment comprises cyclosporin A, Dow Corning 3225C®, and Orange oil.

Another embodiment comprises cyclosporin A, Dow Corning 5200®, and Orange oil.

Another embodiment comprises cyclosporin A, Dow Corning Q4-3667®, and Orange oil.

Another embodiment comprises cyclosporin A, Monasil PCA®, and Orange oil.

Another embodiment comprises cyclosporin A, Monasil PDM®, and Orange oil.

Another embodiment comprises cyclosporin A, Monasil PLN®, and Orange oil.

Another embodiment comprises cyclosporin A, Polyderm PPI-SI-WS®, and Orange oil.

Another embodiment comprises cyclosporin A, Troysol 380W®, and Orange oil.

Another embodiment comprises cyclosporin A, Troysol S366®, and Orange oil.

Another embodiment comprises cyclosporin A, a Sorbitan Derivative, and Orange oil.

Another embodiment comprises cyclosporin A, Alkamuls SML®, and Orange oil.

Another embodiment comprises cyclosporin A, Alkamuls SMO®, and Orange oil.

Another embodiment comprises cyclosporin A, Alkamuls STO®, and Orange oil.

Another embodiment comprises cyclosporin A, Arlacel 20®, and Orange oil.

Another embodiment comprises cyclosporin A, Arlacel 40®, and Orange oil.

Another embodiment comprises cyclosporin A, Arlacel 60®, and Orange oil.

Another embodiment comprises cyclosporin A, Arlacel 80®, and Orange oil.

Another embodiment comprises cyclosporin A, Arlacel C®, and Orange oil.

Another embodiment comprises cyclosporin A, Armul 21®, and Orange oil.

Another embodiment comprises cyclosporin A, Atlox 80®, and Orange oil.

Another embodiment comprises cyclosporin A, Atlox 847®, and Orange oil.

Another embodiment comprises cyclosporin A, Atlox 1045A®, and Orange oil.

Another embodiment comprises cyclosporin A, Canarcel 20®, and Orange oil.

Another embodiment comprises cyclosporin A, Canarcel 60®, and Orange oil.

Another embodiment comprises cyclosporin A, Canarcel 80®, and Orange oil.

Another embodiment comprises cyclosporin A, Canarcel TW 20®, and Orange oil.

Another embodiment comprises cyclosporin A, Canarcel TW 60®, and Orange oil.

Another embodiment comprises cyclosporin A, Canarcel TW 80®, and Orange oil.

Another embodiment comprises cyclosporin A, Coladet BSB-P®, and Orange oil.

Another embodiment comprises cyclosporin A, Customulse O-20®, and Orange oil.

Another embodiment comprises cyclosporin A, Dehymuls E®, and Orange oil.

Another embodiment comprises cyclosporin A, DeSotan SMO®, and Orange oil.

Another embodiment comprises cyclosporin A, DeSotan SMO-20®, and Orange oil.

Another embodiment comprises cyclosporin A, DeSotan SMT®, and Orange oil.

Another embodiment comprises cyclosporin A, DeSotan SMT-20®, and Orange oil.

Another embodiment comprises cyclosporin A, Durfax 60®, and Orange oil.

Another embodiment comprises cyclosporin A, Durfax 65®, and Orange oil.

Another embodiment comprises cyclosporin A, Durfax 80®, and Orange oil.

Another embodiment comprises cyclosporin A, Durtan 60®, and Orange oil.

Another embodiment comprises cyclosporin A, Durtan 65®, and Orange oil.

Another embodiment comprises cyclosporin A, Liposorb L®, and Orange oil.

Another embodiment comprises cyclosporin A, Liposorb L-10®, and Orange oil.

Another embodiment comprises cyclosporin A, Liposorb L-20®, and Orange oil.

Another embodiment comprises cyclosporin A, Liposorb O®, and Orange oil.

Another embodiment comprises cyclosporin A, Liposorb O-20®, and Orange oil.

Another embodiment comprises cyclosporin A, Liposorb P®, and Orange oil.

Another embodiment comprises cyclosporin A, Liposorb P-20®, and Orange oil.

Another embodiment comprises cyclosporin A, Liposorb S®, and Orange oil.

Another embodiment comprises cyclosporin A, Liposorb S-20®, and Orange oil.

Another embodiment comprises cyclosporin A, Liposorb SQO®, and Orange oil.

Another embodiment comprises cyclosporin A, Liposorb TO®, and Orange oil.

Another embodiment comprises cyclosporin A, Liposorb TS®, and Orange oil.

Another embodiment comprises cyclosporin A, Liposorb TS-20®, and Orange oil.

Another embodiment comprises cyclosporin A, Lumisorb PS®, and Orange oil.

Another embodiment comprises cyclosporin A, Lumisorb SMO (T)®, and Orange oil.

Another embodiment comprises cyclosporin A, Lumisorb SMS K®, and Orange oil.

Another embodiment comprises cyclosporin A, Lumisorb SSO®, and Orange oil.

Another embodiment comprises cyclosporin A, Lumisorb STS K®, and Orange oil.

Another embodiment comprises cyclosporin A, Lumisorb STT®, and Orange oil.

Another embodiment comprises cyclosporin A, Magrabar SMO®, and Orange oil.

Another embodiment comprises cyclosporin A, Magrabar SMO-VEG®, and Orange oil.

Another embodiment comprises cyclosporin A, Magrabar SMT®, and Orange oil.

Another embodiment comprises cyclosporin A, Magrabar STO®, and Orange oil.

Another embodiment comprises cyclosporin A, Miracare BC-27®, and Orange oil.

Another embodiment comprises cyclosporin A, Ritabate 20®, and Orange oil.

Another embodiment comprises cyclosporin A, Ritabate 40®, and Orange oil.

Another embodiment comprises cyclosporin A, Ritabate 60®, and Orange oil.

Another embodiment comprises cyclosporin A, Ritabate 80®, and Orange oil.

Another embodiment comprises cyclosporin A, T-Maz®, and Orange oil.

Another embodiment comprises cyclosporin A, Tego SML®, and Orange oil.

Another embodiment comprises cyclosporin A, Tego SML 20®, and Orange oil.

Another embodiment comprises cyclosporin A, Tego SMO 80 V®, and Orange oil.

Another embodiment comprises cyclosporin A, Tego SMO V®, and Orange oil.

Another embodiment comprises cyclosporin A, Tego SMS®, and Orange oil.

Another embodiment comprises cyclosporin A, Tego STO V®, and Orange oil.

Another embodiment comprises cyclosporin A, Tween 21®, and Orange oil.

Another embodiment comprises cyclosporin A, Tween 40®, and Orange oil.

Another embodiment comprises cyclosporin A, Tween 60®, and Orange oil.

Another embodiment comprises cyclosporin A, Tween 60 K®, and Orange oil.

Another embodiment comprises cyclosporin A, Tween 61®, and Orange oil.

Another embodiment comprises cyclosporin A, Tween 65®, and Orange oil.

Another embodiment comprises cyclosporin A, Tween 80®, and Orange oil.

Another embodiment comprises cyclosporin A, Tween 80 K®, and Orange oil.

Another embodiment comprises cyclosporin A, Tween 81®, and Orange oil.

Another embodiment comprises cyclosporin A, Tween 85®, and Orange oil.

Another embodiment comprises cyclosporin A, a Sucrose or Glucose Ester, or Derivative thereof, and Orange oil.

Another embodiment comprises cyclosporin A, DeSulf GOS-P-60WCG®, and Orange oil.

Another embodiment comprises cyclosporin A, Glucam E-20 Distearate®, and Orange oil.

Another embodiment comprises cyclosporin A, Glucamate DOE-120®, and Orange oil.

Another embodiment comprises cyclosporin A, Glucamate SSE-20®, and Orange oil.

Another embodiment comprises cyclosporin A, Glucate DO®, and Orange oil.

Another embodiment comprises cyclosporin A, Glucate SS®, and Orange oil.

Another embodiment comprises cyclosporin A, Glucopon 425 UP®, and Orange oil.

Another embodiment comprises cyclosporin A, Isolan IS®, and Orange oil.

Another embodiment comprises cyclosporin A, Mazon 40®, and Orange oil.

Another embodiment comprises cyclosporin A, Montanov 82®, and Orange oil.

Another embodiment comprises cyclosporin A, Montanov 202®, and Orange oil.

Another embodiment comprises cyclosporin A, Montanov S®, and Orange oil.

Another embodiment comprises cyclosporin A, Rheozan®, and Orange oil.

Another embodiment comprises cyclosporin A, Simulsol AS 48®, and Orange oil.

Another embodiment comprises cyclosporin A, Simulsol SL 4®, and Orange oil.

Another embodiment comprises cyclosporin A, Simulsol SL 10®, and Orange oil.

Another embodiment comprises cyclosporin A, Simulsol SL 11W®, and Orange oil.

Another embodiment comprises cyclosporin A, Simulsol SL 55®, and Orange oil.

Another embodiment comprises cyclosporin A, Suga Nate 100 and 160®, and Orange oil.

Another embodiment comprises cyclosporin A, Tego Care 450®, and Orange oil.

Another embodiment comprises cyclosporin A, Tego Care CG 90®, and Orange oil.

Another embodiment comprises cyclosporin A, Tego Care PS®, and Orange oil.

Another embodiment comprises cyclosporin A, Tegosoft PSE 141 G®, and Orange oil.

Another embodiment comprises cyclosporin A, Tegotens G 826®, and Orange oil.

Another embodiment comprises cyclosporin A, Triton BG-10 (70%)®, and Orange oil.

Another embodiment comprises cyclosporin A, Triton CG-110 (60%)®, and Orange oil.

Another embodiment comprises cyclosporin A, Wickenol 545®, and Orange oil.

Another embodiment comprises cyclosporin A, an alcohol, and Clary sage oil.

Another embodiment comprises cyclosporin A, Diglycerol®, and Clary sage oil.

Another embodiment comprises cyclosporin A, Hetoxide GT-80®, and Clary sage oil.

Another embodiment comprises cyclosporin A, Lexemul BEO®, and Clary sage oil.

Another embodiment comprises cyclosporin A, Polyglycerol-3®, and Clary sage oil.

Another embodiment comprises cyclosporin A, Redicote E Series®, and Clary sage oil.

Another embodiment comprises cyclosporin A, Simulsol OX 1005L®, and Clary sage oil.

Another embodiment comprises cyclosporin A, Stanfax 567®, and Clary sage oil.

Another embodiment comprises cyclosporin A, TA-1618®, and Clary sage oil.

Another embodiment comprises cyclosporin A, Witconol H-31A®, and Clary sage oil.

Another embodiment comprises cyclosporin A, an amine oxide, and Clary sage oil.

Another embodiment comprises cyclosporin A, AO-405®, and Clary sage oil.

Another embodiment comprises cyclosporin A, AO-455®, and Clary sage oil.

Another embodiment comprises cyclosporin A, AO 728 Special®, and Clary sage oil.

Another embodiment comprises cyclosporin A, Barlox 12®, and Clary sage oil.

Another embodiment comprises cyclosporin A, Barlox 14®, and Clary sage oil.

Another embodiment comprises cyclosporin A, Burcoxide Lo®, and Clary sage oil.

Another embodiment comprises cyclosporin A, Caloxamine LO®, and Clary sage oil.

Another embodiment comprises cyclosporin A, Chemoxide CAW®, and Clary sage oil.

Another embodiment comprises cyclosporin A, Chemoxide LM-30®, and Clary sage oil.

Another embodiment comprises cyclosporin A, Chemoxide LO®, and Clary sage oil.

Another embodiment comprises cyclosporin A, Chemoxide MO®, and Clary sage oil.

Another embodiment comprises cyclosporin A, Colalux CAO-35®, and Clary sage oil.

Another embodiment comprises cyclosporin A, Colalux LO®, and Clary sage oil.

Another embodiment comprises cyclosporin A, DeMox CAPO®, and Clary sage oil.

Another embodiment comprises cyclosporin A, DeMox CSG-30®, and Clary sage oil.

Another embodiment comprises cyclosporin A, DeMox LAO®, and Clary sage oil.

Another embodiment comprises cyclosporin A, Emcol L®, and Clary sage oil.

Another embodiment comprises cyclosporin A, Empigen OB®, and Clary sage oil.

Another embodiment comprises cyclosporin A, Empigen OS/A®, and Clary sage oil.

Another embodiment comprises cyclosporin A, Foamox CDO®, and Clary sage oil.

Another embodiment comprises cyclosporin A, Foamox DMM®, and Clary sage oil.

Another embodiment comprises cyclosporin A, Foamox DMS®, and Clary sage oil.

Another embodiment comprises cyclosporin A, Genaminox KC®, and Clary sage oil.

Another embodiment comprises cyclosporin A, Genaminox LA®, and Clary sage oil.

Another embodiment comprises cyclosporin A, Hartofoam SAO®, and Clary sage oil.

Another embodiment comprises cyclosporin A, Hartox DMCD®, and Clary sage oil.

Another embodiment comprises cyclosporin A, Lipowax DAT®, and Clary sage oil.

Another embodiment comprises cyclosporin A, Lipowax PB Pastilles®, and Clary sage oil.

Another embodiment comprises cyclosporin A, Mackamine C8®, and Clary sage oil.

Another embodiment comprises cyclosporin A, Mackamine C10®, and Clary sage oil.

Another embodiment comprises cyclosporin A, Mackamine C14®, and Clary sage oil.

Another embodiment comprises cyclosporin A, Mackamine CAO®, and Clary sage oil.

Another embodiment comprises cyclosporin A, Mackamine CO®, and Clary sage oil.

Another embodiment comprises cyclosporin A, Mackamine LO®, and Clary sage oil.

Another embodiment comprises cyclosporin A, Mackamine O2®, and Clary sage oil.

Another embodiment comprises cyclosporin A, Mackamine SAO®, and Clary sage oil.

Another embodiment comprises cyclosporin A, Mackamine SO®, and Clary sage oil.

Another embodiment comprises cyclosporin A, Mazox KCAO®, and Clary sage oil.

Another embodiment comprises cyclosporin A, Monalac MO®, and Clary sage oil.

Another embodiment comprises cyclosporin A, Norfox LDA®, and Clary sage oil.

Another embodiment comprises cyclosporin A, Rhodamox LO®, and Clary sage oil.

Another embodiment comprises cyclosporin A, Schercamox C-AA®, and Clary sage oil.

Another embodiment comprises cyclosporin A, Schercamox DMA®, and Clary sage oil.

Another embodiment comprises cyclosporin A, Schercamox DML®, and Clary sage oil.

Another embodiment comprises cyclosporin A, Schercamox DMM®, and Clary sage oil.

Another embodiment comprises cyclosporin A, Schercamox DMS®, and Clary sage oil.

Another embodiment comprises cyclosporin A, Tegotens DO®, and Clary sage oil.

Another embodiment comprises cyclosporin A, Tomah AO-14-2®, and Clary sage oil.

Another embodiment comprises cyclosporin A, Triaminox CDO®, and Clary sage oil.

Another embodiment comprises cyclosporin A, a block polymer, and Clary sage oil.

Another embodiment comprises cyclosporin A, AL 2070®, and Clary sage oil.

Another embodiment comprises cyclosporin A, Antarox 17-R-2®, and Clary sage oil.

Another embodiment comprises cyclosporin A, Antarox 25-R-2®, and Clary sage oil.

Another embodiment comprises cyclosporin A, Antarox 31-R-1®, and Clary sage oil.

Another embodiment comprises cyclosporin A, Antarox P-84®, and Clary sage oil.

Another embodiment comprises cyclosporin A, Antarox P-104/H®, and Clary sage oil.

Another embodiment comprises cyclosporin A, Arnox BP-Series®, and Clary sage oil.

Another embodiment comprises cyclosporin A, Chemonic 435®, and Clary sage oil.

Another embodiment comprises cyclosporin A, Chemonic D-25®, and Clary sage oil.

Another embodiment comprises cyclosporin A, Chemonic PL Series®, and Clary sage oil.

Another embodiment comprises cyclosporin A, Ethox L-121®, and Clary sage oil.

Another embodiment comprises cyclosporin A, Ethox L-122®, and Clary sage oil.

Another embodiment comprises cyclosporin A, Genapol PF-10®, and Clary sage oil.

Another embodiment comprises cyclosporin A, Genapol PF-20®, and Clary sage oil.

Another embodiment comprises cyclosporin A, Genapol PF-40A®, and Clary sage oil.

Another embodiment comprises cyclosporin A, Norfox 2-LP®, and Clary sage oil.

Another embodiment comprises cyclosporin A, Simulsol NW 342®, and Clary sage oil.

Another embodiment comprises cyclosporin A, T-Det BP-1®, and Clary sage oil.

Another embodiment comprises cyclosporin A, T-Det XD®, and Clary sage oil.

Another embodiment comprises cyclosporin A, T-Det XH®, and Clary sage oil.

Another embodiment comprises cyclosporin A, Triton CF-32®, and Clary sage oil.

Another embodiment comprises cyclosporin A, Witconol 171®, and Clary sage oil.

Another embodiment comprises cyclosporin A, Witconol 324®, and Clary sage oil.

Another embodiment comprises cyclosporin A, Witconol 324D®, and Clary sage oil.

Another embodiment comprises cyclosporin A, Witconol PD-2000®, and Clary sage oil.

Another embodiment comprises cyclosporin A, Emcol CN-6®, and Clary sage oil.

Another embodiment comprises cyclosporin A, Ethcarb®, and Clary sage oil.

Another embodiment comprises cyclosporin A, Gemtex WNT-Conc®, and. Clary sage oil.

Another embodiment comprises cyclosporin A, Incrodet TD7-C®, and Clary sage oil.

Another embodiment comprises cyclosporin A, Marlinat CM 105/80®, and Clary sage oil.

Another embodiment comprises cyclosporin A, Marlowet 1072®, and Clary sage oil.

Another embodiment comprises cyclosporin A, Marlowet 4530®, and Clary sage oil.

Another embodiment comprises cyclosporin A, Marlowet 4530 LF®, and Clary sage oil.

Another embodiment comprises cyclosporin A, Marlowet 4534®, and Clary sage oil.

Another embodiment comprises cyclosporin A, Marlowet 4538®, and Clary sage oil.

Another embodiment comprises cyclosporin A, Marlowet 4539®, and Clary sage oil.

Another embodiment comprises cyclosporin A, Marlowet 4539 LF®, and Clary sage oil.

Another embodiment comprises cyclosporin A, Marlowet 4541®, and Clary sage oil.

Another embodiment comprises cyclosporin A, Miranate LEC-80®, and Clary sage oil.

Another embodiment comprises cyclosporin A, Sandopan B®, and Clary sage oil.

Another embodiment comprises cyclosporin A, Sandopan B Modified®, and Clary sage oil.

Another embodiment comprises cyclosporin A, Sandopan LS-24 Gel®, and Clary sage oil.

Another embodiment comprises cyclosporin A, Surfine T-A®, and Clary sage oil.

Another embodiment comprises cyclosporin A, a Carboxylic Acid/Fatty Acid, and Clary sage oil.

Another embodiment comprises cyclosporin A, Colaterge RAM®, and Clary sage oil.

Another embodiment comprises cyclosporin A, Colatrope INC®, and Clary sage oil.

Another embodiment comprises cyclosporin A, Crodacid B®, and Clary sage oil.

Another embodiment comprises cyclosporin A, DeTrope CA-100®, and Clary sage oil.

Another embodiment comprises cyclosporin A, Latol MTO®, and Clary sage oil.

Another embodiment comprises cyclosporin A, Lumulse CC-33 K®, and Clary sage oil.

Another embodiment comprises cyclosporin A, Mulls 2218®, and Clary sage oil.

Another embodiment comprises cyclosporin A, OL-600®, and Clary sage oil.

Another embodiment comprises cyclosporin A, OL-800®, and Clary sage oil.

Another embodiment comprises cyclosporin A, R-910®, and Clary sage oil.

Another embodiment comprises cyclosporin A, S-210®, and Clary sage oil.

Another embodiment comprises cyclosporin A, Sandopan DTC Acid®, and Clary sage oil.

Another embodiment comprises cyclosporin A, Sandopan LS 24 N®, and Clary sage oil.

Another embodiment comprises cyclosporin A, Sandopan MA-18®, and Clary sage oil.

Another embodiment comprises cyclosporin A, an Ethoxylated Alcohol, and Clary sage oil.

Another embodiment comprises cyclosporin A, Adsee 799®, and Clary sage oil.

Another embodiment comprises cyclosporin A, Adsee 799®, and Clary sage oil.

Another embodiment comprises cyclosporin A, Alfonic 610-3.5®, and Clary sage oil.

Another embodiment comprises cyclosporin A, Alfonic 810-2®, and Clary sage oil.

Another embodiment comprises cyclosporin A, Alfonic 810-6®, and Clary sage oil.

Another embodiment comprises cyclosporin A, Alfonic 1012-3®, and Clary sage oil.

Another embodiment comprises cyclosporin A, Alfonic 1012-5®, and Clary sage oil.

Another embodiment comprises cyclosporin A, Alfonic 1216CO-1.5®, and Clary sage oil.

Another embodiment comprises cyclosporin A, Alfonic 1216CO-7®, and Clary sage oil.

Another embodiment comprises cyclosporin A, Alfonic 1412-3®, and Clary sage oil.

Another embodiment comprises cyclosporin A, Alfonic 1412-7®, and Clary sage oil.

Another embodiment comprises cyclosporin A, Arlasolve 200®, and Clary sage oil.

Another embodiment comprises cyclosporin A, Arlasolve 200 Liquid®, and Clary sage oil.

Another embodiment comprises cyclosporin A, Armix 180-C®, and Clary sage oil.

Another embodiment comprises cyclosporin A, Armix 183®, and Clary sage oil.

Another embodiment comprises cyclosporin A, Armul 2404®, and Clary sage oil.

Another embodiment comprises cyclosporin A, Armul 2404®, and Clary sage oil.

Another embodiment comprises cyclosporin A, Atlas EMJ-C®, and Clary sage oil.

Another embodiment comprises cyclosporin A, Atlas G-2109®, and Clary sage oil.

Another embodiment comprises cyclosporin A, Atlas G-3886®, and Clary sage oil.

Another embodiment comprises cyclosporin A, Atlas G-3890®, and Clary sage oil.

Another embodiment comprises cyclosporin A, Bio Soft E-200®, and Clary sage oil.

Another embodiment comprises cyclosporin A, Bio Soft E-300®, and Clary sage oil.

Another embodiment comprises cyclosporin A, Bio Soft E-400®, and Clary sage oil.

Another embodiment comprises cyclosporin A, Bio Soft EN 600®, and Clary sage oil.

Another embodiment comprises cyclosporin A, Bio Soft TD-400®, and Clary sage oil.

Another embodiment comprises cyclosporin A, Bio Soft TD-630®, and Clary sage oil.

Another embodiment comprises cyclosporin A, Brij 30®, and Clary sage oil.

Another embodiment comprises cyclosporin A, Brij 52®, and Clary sage oil.

Another embodiment comprises cyclosporin A, Brij 56®, and Clary sage oil.

Another embodiment comprises cyclosporin A, Brij 58®, and Clary sage oil.

Another embodiment comprises cyclosporin A, Brij 72®, and Clary sage oil.

Another embodiment comprises cyclosporin A, Brij 76®, and Clary sage oil.

Another embodiment comprises cyclosporin A, Brij 78®, and Clary sage oil.

Another embodiment comprises cyclosporin A, Brij 93®, and Clary sage oil.

Another embodiment comprises cyclosporin A, Brij 97®, and Clary sage oil.

Another embodiment comprises cyclosporin A, Brij 98®, and Clary sage oil.

Another embodiment comprises cyclosporin A, Brij 700®, and Clary sage oil.

Another embodiment comprises cyclosporin A, Brij 700 S®, and Clary sage oil.

Another embodiment comprises cyclosporin A, Brij 721®, and Clary sage oil.

Another embodiment comprises cyclosporin A, Brij 721 S®, and Clary sage oil.

Another embodiment comprises cyclosporin A, Burcoterge CDG®, and Clary sage oil.

Another embodiment comprises cyclosporin A, Canasol AT 600®, and Clary sage oil.

Another embodiment comprises cyclosporin A, Canasol AT 800®, and Clary sage oil.

Another embodiment comprises cyclosporin A, Canasol AT 1200®, and Clary sage oil.

Another embodiment comprises cyclosporin A, Canasol BJ 35®, and Clary sage oil.

Another embodiment comprises cyclosporin A, Canasol BJ 36®, and Clary sage oil.

Another embodiment comprises cyclosporin A, Canasol BJ 52®, and Clary sage oil.

Another embodiment comprises cyclosporin A, Canasol BJ 58®, and Clary sage oil.

Another embodiment comprises cyclosporin A, Canasol BJ 72®, and Clary sage oil.

Another embodiment comprises cyclosporin A, Canasol BJ 78®, and Clary sage oil.

Another embodiment comprises cyclosporin A, Canasol BJ 98®, and Clary sage oil.

Another embodiment comprises cyclosporin A, Canasol BJ 307®, and Clary sage oil.

Another embodiment comprises cyclosporin A, Cerfak 1400®, and Clary sage oil.

Another embodiment comprises cyclosporin A, Cetomacrogol 1000 BP®, and Clary sage oil.

Another embodiment comprises cyclosporin A, Chemonic C-2®, and Clary sage oil.

Another embodiment comprises cyclosporin A, Chemonic C-10®, and Clary sage oil.

Another embodiment comprises cyclosporin A, Chemonic C-20®, and Clary sage oil.

Another embodiment comprises cyclosporin A, Chemonic CT-12®, and Clary sage oil.

Another embodiment comprises cyclosporin A, Chemonic CT-20®, and Clary sage oil.

Another embodiment comprises cyclosporin A, Chemonic CT-30®, and Clary sage oil.

Another embodiment comprises cyclosporin A, Chemonic CT-55®, and Clary sage oil.

Another embodiment comprises cyclosporin A, Chemonic G-7®, and Clary sage oil.

Another embodiment comprises cyclosporin A, Chemonic G-26®, and Clary sage oil.

Another embodiment comprises cyclosporin A, Chemonic L-4®, and Clary sage oil.

Another embodiment comprises cyclosporin A, Chemonic L-7®, and Clary sage oil.

Another embodiment comprises cyclosporin A, Chemonic L-12®, and Clary sage oil.

Another embodiment comprises cyclosporin A, Chemonic L-23®, and Clary sage oil.

Another embodiment comprises cyclosporin A, Chemonic O-2®, and Clary sage oil.

Another embodiment comprises cyclosporin A, Chemonic O-5®, and Clary sage oil.

Another embodiment comprises cyclosporin A, Chemonic O-10®, and Clary sage oil.

Another embodiment comprises cyclosporin A, Chemonic O-20®, and Clary sage oil.

Another embodiment comprises cyclosporin A, Chemonic S-2®, and Clary sage oil.

Another embodiment comprises cyclosporin A, Chemonic S-10®, and Clary sage oil.

Another embodiment comprises cyclosporin A, Chemonic S-20®, and Clary sage oil.

Another embodiment comprises cyclosporin A, Colamulse FE®, and Clary sage oil.

Another embodiment comprises cyclosporin A, Cremophor A 20®, and Clary sage oil.

Another embodiment comprises cyclosporin A, Cremophor SA 2®, and Clary sage oil.

Another embodiment comprises cyclosporin A, Dehydol 100®, and Clary sage oil.

Another embodiment comprises cyclosporin A, Dehydol O-4®, and Clary sage oil.

Another embodiment comprises cyclosporin A, Delonic C-18®, and Clary sage oil.

Another embodiment comprises cyclosporin A, DeSonic 6T®, and Clary sage oil.

Another embodiment comprises cyclosporin A, DeSonic 9D®, and Clary sage oil.

Another embodiment comprises cyclosporin A, DeSonic 9T®, and Clary sage oil.

Another embodiment comprises cyclosporin A, DeSonic 12D®, and Clary sage oil.

Another embodiment comprises cyclosporin A, DeSonic 12T®, and Clary sage oil.

Another embodiment comprises cyclosporin A, DeSonic 15T®, and Clary sage oil.

Another embodiment comprises cyclosporin A, DeSonic TDA-9®, and Clary sage oil.

Another embodiment comprises cyclosporin A, DeThox GLG-7®, and Clary sage oil.

Another embodiment comprises cyclosporin A, DeThox GLG-26®, and Clary sage oil.

Another embodiment comprises cyclosporin A, DeThox LA-4®, and Clary sage oil.

Another embodiment comprises cyclosporin A, DeThox LA-23®, and Clary sage oil.

Another embodiment comprises cyclosporin A, DeThox SA-80®, and Clary sage oil.

Another embodiment comprises cyclosporin A, Disponil O5®, and Clary sage oil.

Another embodiment comprises cyclosporin A, Eccoterge EO-41B®, and Clary sage oil.

Another embodiment comprises cyclosporin A, Empilan KA2.5/90®, and Clary sage oil.

Another embodiment comprises cyclosporin A, Empilan KA5/90®, and Clary sage oil.

Another embodiment comprises cyclosporin A, Empilan KM-20®, and Clary sage oil.

Another embodiment comprises cyclosporin A, Empilan KM-50®, and Clary sage oil.

Another embodiment comprises cyclosporin A, Empilan L-23®, and Clary sage oil.

Another embodiment comprises cyclosporin A, Ethylan 25-3®, and Clary sage oil.

Another embodiment comprises cyclosporin A, Ethylan 1204®, and Clary sage oil.

Another embodiment comprises cyclosporin A, Ethylan DA-4®, and Clary sage oil.

Another embodiment comprises cyclosporin A, Ethylan LA-230®, and Clary sage oil.

Another embodiment comprises cyclosporin A, Ethylan SN®, and Clary sage oil.

Another embodiment comprises cyclosporin A, Ethylan TD-60®, and Clary sage oil.

Another embodiment comprises cyclosporin A, Ethylan TD-100®, and Clary sage oil.

Another embodiment comprises cyclosporin A, Ethylan TD-1407®, and Clary sage oil.

Another embodiment comprises cyclosporin A, Eumulgin B1®, and Clary sage oil.

Another embodiment comprises cyclosporin A, Eumulgin B2®, and Clary sage oil.

Another embodiment comprises cyclosporin A, Eumulgin B3®, and Clary sage oil.

Another embodiment comprises cyclosporin A, Eumulgin O-10®, and Clary sage oil.

Another embodiment comprises cyclosporin A, Flo Mo 80/20®, and Clary sage oil.

Another embodiment comprises cyclosporin A, Flo Mo Low Foam®, and Clary sage oil.

Another embodiment comprises cyclosporin A, Forlan C-24®, and Clary sage oil.

Another embodiment comprises cyclosporin A, Genapol 1454®, and Clary sage oil.

Another embodiment comprises cyclosporin A, Genapol BA-020®, and Clary sage oil.

Another embodiment comprises cyclosporin A, Genapol BA-040®, and Clary sage oil.

Another embodiment comprises cyclosporin A, Genapol C-100®, and Clary sage oil.

Another embodiment comprises cyclosporin A, Genapol DA 060®, and Clary sage oil.

Another embodiment comprises cyclosporin A, Genapol HS 020®, and Clary sage oil.

Another embodiment comprises cyclosporin A, Genapol HS 200®, and Clary sage oil.

Another embodiment comprises cyclosporin A, Genapol ID-040®, and Clary sage oil.

Another embodiment comprises cyclosporin A, Genapol ID-060®, and Clary sage oil.

Another embodiment comprises cyclosporin A, Genapol ID-090®, and Clary sage oil.

Another embodiment comprises cyclosporin A, Genapol LA 010®, and Clary sage oil.

Another embodiment comprises cyclosporin A, Genapol LA 020®, and Clary sage oil.

Another embodiment comprises cyclosporin A, Genapol LA 030®, and Clary sage oil.

Another embodiment comprises cyclosporin A, Genapol LA 040®, and Clary sage oil.

Another embodiment comprises cyclosporin A, Genapol LA 050®, and Clary sage oil.

Another embodiment comprises cyclosporin A, Genapol LA 060®, and Clary sage oil.

Another embodiment comprises cyclosporin A, Genapol LA 070®, and Clary sage oil.

Another embodiment comprises cyclosporin A, Genapol LA 070S®, and Clary sage oil.

Another embodiment comprises cyclosporin A, Genapol LA 230®, and Clary sage oil.

Another embodiment comprises cyclosporin A, Genapbl O 020®, and Clary sage oil.

Another embodiment comprises cyclosporin A, Genapol O 050®, and Clary sage oil.

Another embodiment comprises cyclosporin A, Genapol O 100®, and Clary sage oil.

Another embodiment comprises cyclosporin A, Genapol O 200®, and Clary sage oil.

Another embodiment comprises cyclosporin A, Genapol SA 030®, and Clary sage oil.

Another embodiment comprises cyclosporin A, Genapol SA 120®, and Clary sage oil.

Another embodiment comprises cyclosporin A, Genapol T-020®, and Clary sage oil.

Another embodiment comprises cyclosporin A, Genapol UD-030®, and Clary sage oil.

Another embodiment comprises cyclosporin A, Genapol UD-050®, and Clary sage oil.

Another embodiment comprises cyclosporin A, Genapol UD-070®, and Clary sage oil.

Another embodiment comprises cyclosporin A, Genapol UD-079®, and Clary sage oil.

Another embodiment comprises cyclosporin A, Genapol UD-080®, and Clary sage oil.

Another embodiment comprises cyclosporin A, Genapol UD-110®, and Clary sage oil.

Another embodiment comprises cyclosporin A, Genapol X 030®, and Clary sage oil.

Another embodiment comprises cyclosporin A, Genapol X 050®, and Clary sage oil.

Another embodiment comprises cyclosporin A, Genapol X 060®, and Clary sage oil.

Another embodiment comprises cyclosporin A, Genapol X 070®, and Clary sage oil.

Another embodiment comprises cyclosporin A, Genapol X 080®, and Clary sage oil.

Another embodiment comprises cyclosporin A, Genapol X 100®, and Clary sage oil.

Another embodiment comprises cyclosporin A, Genapol X159®, and Clary sage oil.

Another embodiment comprises cyclosporin A, Generol 122 E5®, and Clary sage oil.

Another embodiment comprises cyclosporin A, Generol 122 E25®, and Clary sage oil.

Another embodiment comprises cyclosporin A, Hostacerin T-3®, and Clary sage oil.

Another embodiment comprises cyclosporin A, Iconol LF 110®, and Clary sage oil.

Another embodiment comprises cyclosporin A, Incropol CS-20®, and Clary sage oil.

Another embodiment comprises cyclosporin A, Lexemul CS-20®, and Clary sage oil.

Another embodiment comprises cyclosporin A, Liponic EG-1®, and Clary sage oil.

Another embodiment comprises cyclosporin A, Lipowax D®, and Clary sage oil.

Another embodiment comprises cyclosporin A, Lipowax G®, and Clary sage oil.

Another embodiment comprises cyclosporin A, Lipowax NI®, and Clary sage oil.

Another embodiment comprises cyclosporin A, Lipowax P®, and Clary sage oil.

Another embodiment comprises cyclosporin A, Lipowax P-31®, and Clary sage oil.

Another embodiment comprises cyclosporin A, Lipowax PR®, and Clary sage oil.

Another embodiment comprises cyclosporin A, Lumulse CS-20®, and Clary sage oil.

Another embodiment comprises cyclosporin A, Macol CSA-20®, and Clary sage oil.

Another embodiment comprises cyclosporin A, Marlox B 24/50®, and Clary sage oil.

Another embodiment comprises cyclosporin A, Mazawet 77®, and Clary sage oil.

Another embodiment comprises cyclosporin A, Norfox 1713®, and Clary sage oil.

Another embodiment comprises cyclosporin A, Norfox 2579®, and Clary sage oil.

Another embodiment comprises cyclosporin A, Norfox Lo Foam®, and Clary sage oil.

Another embodiment comprises cyclosporin A, Promulgen D®, and Clary sage oil.

Another embodiment comprises cyclosporin A, Promulgen G®, and Clary sage oil.

Another embodiment comprises cyclosporin A, Renex 30®, and Clary sage oil.

Another embodiment comprises cyclosporin A, Renex 36®, and Clary sage oil.

Another embodiment comprises cyclosporin A, Rhodasurf A 24®, and Clary sage oil.

Another embodiment comprises cyclosporin A, Rhodasurf AAE-10®, and Clary sage oil.

Another embodiment comprises cyclosporin A, Rhodasurf BEH-25®, and Clary sage oil.

Another embodiment comprises cyclosporin A, Rhodasurf BEH-40®, and Clary sage oil.

Another embodiment comprises cyclosporin A, Rhodasurf DA 530®, and Clary sage oil.

Another embodiment comprises cyclosporin A, Rhodasurf DA 630®, and Clary sage oil.

Another embodiment comprises cyclosporin A, Rhodasurf DA 639®, and Clary sage oil.

Another embodiment comprises cyclosporin A, Rhodasurf LAN-23®, and Clary sage oil.

Another embodiment comprises cyclosporin A, Rhodasurf ON-870®, and Clary sage oil.

Another embodiment comprises cyclosporin A, Rhodasurf ON-877®, and Clary sage oil.

Another embodiment comprises cyclosporin A, Rhodasurf TB-970 FLK®, and Clary sage oil.

Another embodiment comprises cyclosporin A, Ritacet-20®, and Clary sage oil.

Another embodiment comprises cyclosporin A, Ritachol 1000®, and Clary sage oil.

Another embodiment comprises cyclosporin A, Ritachol 2000®, and Clary sage oil.

Another embodiment comprises cyclosporin A, Ritachol 5000®, and Clary sage oil.

Another embodiment comprises cyclosporin A, Ritox 35®, and Clary sage oil.

Another embodiment comprises cyclosporin A, Surfonic DA-4®, and Clary sage oil.

Another embodiment comprises cyclosporin A, Surfonic DA-6®, and Clary sage oil.

Another embodiment comprises cyclosporin A, Surfonic L46-7®, and Clary sage oil.

Another embodiment comprises cyclosporin A, Surfonic POA®, and Clary sage oil.

Another embodiment comprises cyclosporin A, Synthrapol KB®, and Clary sage oil.

Another embodiment comprises cyclosporin A, Teginacid®, and Clary sage oil.

Another embodiment comprises cyclosporin A, Teginacid C®, and Clary sage oil.

Another embodiment comprises cyclosporin A, Tegotens EC 11®, and Clary sage oil.

Another embodiment comprises cyclosporin A, Tinegal NA®, and Clary sage oil.

Another embodiment comprises cyclosporin A, Tomadol 400®, and Clary sage oil.

Another embodiment comprises cyclosporin A, Tomadol 600®, and Clary sage oil.

Another embodiment comprises cyclosporin A, Tomadol 900®, and Clary sage oil.

Another embodiment comprises cyclosporin A, Uniperol O®, and Clary sage oil.

Another embodiment comprises cyclosporin A, Witconol SN Series®, and Clary sage oil.

Another embodiment comprises cyclosporin A, an Ethoxylated Alkylphenol, and Clary sage oil.

Another embodiment comprises cyclosporin A, Antarox LF-222®, and Clary sage oil.

Another embodiment comprises cyclosporin A, Atlox 775®, and Clary sage oil.

Another embodiment comprises cyclosporin A, Caloxylate N-9®, and Clary sage oil.

Another embodiment comprises cyclosporin A, Canasol NF-1000®, and Clary sage oil.

Another embodiment comprises cyclosporin A, Canasol NF-3000®, and Clary sage oil.

Another embodiment comprises cyclosporin A, Canasol NF-3070®, and Clary sage oil.

Another embodiment comprises cyclosporin A, Canasol OF 1670®, and Clary sage oil.

Another embodiment comprises cyclosporin A, Canasol OF 2570®, and Clary sage oil.

Another embodiment comprises cyclosporin A, Canasol OF 4070®, and Clary sage oil.

Another embodiment comprises cyclosporin A, Chemax DNP-8®, and Clary sage oil.

Another embodiment comprises cyclosporin A, Chemax DNP-18®, and Clary sage oil.

Another embodiment comprises cyclosporin A, Chemax DNP-150/50®, and Clary sage oil.

Another embodiment comprises cyclosporin A, DeSonic 1.5N®, and Clary sage oil.

Another embodiment comprises cyclosporin A, DeSonic 4N®, and Clary sage oil.

Another embodiment comprises cyclosporin A, DeSonic 5N®, and Clary sage oil.

Another embodiment comprises cyclosporin A, DeSonic 6D®, and Clary sage oil.

Another embodiment comprises cyclosporin A, DeSonic 6N®, and Clary sage oil.

Another embodiment comprises cyclosporin A, DeSonic 7N®, and Clary sage oil.

Another embodiment comprises cyclosporin A, DeSonic 9N®, and Clary sage oil.

Another embodiment comprises cyclosporin A, DeSonic 10D®, and Clary sage oil.

Another embodiment comprises cyclosporin A, DeSonic 11N®, and Clary sage oil.

Another embodiment comprises cyclosporin A, DeSonic 12N®, and Clary sage oil.

Another embodiment comprises cyclosporin A, DeSonic 15N®, and Clary sage oil.

Another embodiment comprises cyclosporin A, DeSonic 20N®, and Clary sage oil.

Another embodiment comprises cyclosporin A, Eccoscour RC®, and Clary sage oil.

Another embodiment comprises cyclosporin A, Eccoterge EO-100®, and Clary sage oil.

Another embodiment comprises cyclosporin A, Emulsifier 632/90%®, and Clary sage oil.

Another embodiment comprises cyclosporin A, Geronol AG-821®, and Clary sage oil.

Another embodiment comprises cyclosporin A, Gradonic N-95®, and Clary sage oil.

Another embodiment comprises cyclosporin A, Hetoxide NP-4®, and Clary sage oil.

Another embodiment comprises cyclosporin A, Hetoxide NP-30®, and Clary sage oil.

Another embodiment comprises cyclosporin A, Hostapal N-100®, and Clary sage oil.

Another embodiment comprises cyclosporin A, Hostapal N-110®, and Clary sage oil.

Another embodiment comprises cyclosporin A, Igepal CTA-639W®, and Clary sage oil.

Another embodiment comprises cyclosporin A, Igepal DAP-9®, and Clary sage oil.

Another embodiment comprises cyclosporin A, Igepal OD-410®, and Clary sage oil.

Another embodiment comprises cyclosporin A, Igepal SS-837®, and Clary sage oil.

Another embodiment comprises cyclosporin A, Lipocol NP-9 USP®, and Clary sage oil.

Another embodiment comprises cyclosporin A, Macol DNP-10®, and Clary sage oil.

Another embodiment comprises cyclosporin A, Marlophen NP 5®, and Clary sage oil.

Another embodiment comprises cyclosporin A, Marlophen P 1®, and Clary sage oil.

Another embodiment comprises cyclosporin A, Surfonic NB®, and Clary sage oil.

Another embodiment comprises cyclosporin A, Surfonic OPB-307®, and Clary sage oil.

Another embodiment comprises cyclosporin A, Surfonic OPB-407®, and Clary sage oil.

Another embodiment comprises cyclosporin A, Syn Fac 334®, and Clary sage oil.

Another embodiment comprises cyclosporin A, Syn Fac 8216®, and Clary sage oil.

Another embodiment comprises cyclosporin A, Triton N-57®, and Clary sage oil.

Another embodiment comprises cyclosporin A, Trycol 6956®, and Clary sage oil.

Another embodiment comprises cyclosporin A, Trycol 6961®, and Clary sage oil.

Another embodiment comprises cyclosporin A, Trycol 6964®, and Clary sage oil.

Another embodiment comprises cyclosporin A, Trycol 6969®, and Clary sage oil.

Another embodiment comprises cyclosporin A, Trycol 6974®, and Clary sage oil.

Another embodiment comprises cyclosporin A, Witbreak DRB-127®, and Clary sage oil.

Another embodiment comprises cyclosporin A, Witbreak DRB-127®, and Clary sage oil.

Another embodiment comprises cyclosporin A, Witconol NP Series®, and Clary sage oil.

Another embodiment comprises cyclosporin A, an Ethoxylated Aryl Phenol, and Clary sage oil.

Another embodiment comprises cyclosporin A, Soprophor BSU®, and Clary sage oil.

Another embodiment comprises cyclosporin A, Soprophor CY/8®, and Clary sage oil.

Another embodiment comprises cyclosporin A, Soprophor S/25®, and Clary sage oil.

Another embodiment comprises cyclosporin A, Witconol NIO®, and Clary sage oil.

Another embodiment comprises cyclosporin A, Witconol NIW®, and Clary sage oil.

Another embodiment comprises cyclosporin A, Witconol S-100®, and Clary sage oil.

Another embodiment comprises cyclosporin A, an Ethoxylated Fatty Acid, and Clary sage oil.

Another embodiment comprises cyclosporin A, Aldo PGHMS®, and Clary sage oil.

Another embodiment comprises cyclosporin A, Alkamuls TO-15/HR®, and Clary sage oil.

Another embodiment comprises cyclosporin A, Armotan AL-69-66®, and Clary sage oil.

Another embodiment comprises cyclosporin A, Cerasynt 840®, and Clary sage oil.

Another embodiment comprises cyclosporin A, Cerasynt 945®, and Clary sage oil.

Another embodiment comprises cyclosporin A, Crystal Inhibitor No. 5®, and Clary sage oil.

Another embodiment comprises cyclosporin A, DeThox Acid L-9®, and Clary sage oil.

Another embodiment comprises cyclosporin A, DeThox Acid S-8®, and Clary sage oil.

Another embodiment comprises cyclosporin A, Ethofat 242/25®, and Clary sage oil.

Another embodiment comprises cyclosporin A, Hydropalat 65®, and Clary sage oil.

Another embodiment comprises cyclosporin A, Lipo EGMS®, and Clary sage oil.

Another embodiment comprises cyclosporin A, Lipopeg 2 DL®, and Clary sage oil.

Another embodiment comprises cyclosporin A, Lipopeg 4 DL®, and Clary sage oil.

Another embodiment comprises cyclosporin A, Lipopeg 4-L®, and Clary sage oil.

Another embodiment comprises cyclosporin A, Lipopeg 39-S®, and Clary sage oil.

Another embodiment comprises cyclosporin A, Lipopeg 4-S®, and Clary sage oil.

Another embodiment comprises cyclosporin A, Lipopeg 10-S®, and Clary sage oil.

Another embodiment comprises cyclosporin A, Lipopeg 100-S®, and Clary sage oil.

Another embodiment comprises cyclosporin A, Lipopeg 6000 DS®, and Clary sage oil.

Another embodiment comprises cyclosporin A, Lumulse 40-L®, and Clary sage oil.

Another embodiment comprises cyclosporin A, Lumulse 40-S®, and Clary sage oil.

Another embodiment comprises cyclosporin A, Lumulse 42-L®, and Clary sage oil.

Another embodiment comprises cyclosporin A, Lumulse 42-S®, and Clary sage oil.

Another embodiment comprises cyclosporin A, Lumulse 100-S®, and Clary sage oil.

Another embodiment comprises cyclosporin A, Lumulse 602-S®, and Clary sage oil.

Another embodiment comprises cyclosporin A, Magrabar PGE-20-0®, and Clary sage oil.

Another embodiment comprises cyclosporin A, Magrabar PGE-20L®, and Clary sage oil.

Another embodiment comprises cyclosporin A, Magrabar PGE-20T®, and Clary sage oil.

Another embodiment comprises cyclosporin A, Magrabar PGE-22-0®, and Clary sage oil.

Another embodiment comprises cyclosporin A, Magrabar PGE-22L®, and Clary sage oil.

Another embodiment comprises cyclosporin A, Magrabar PGE-22T®, and Clary sage oil.

Another embodiment comprises cyclosporin A, Magrabar PGE-40-0®, and Clary sage oil.

Another embodiment comprises cyclosporin A, Magrabar PGE-40L®, and Clary sage oil.

Another embodiment comprises cyclosporin A, Magrabar PGE-40T®, and Clary sage oil.

Another embodiment comprises cyclosporin A, Magrabar PGE-42-0®, and Clary sage oil.

Another embodiment comprises cyclosporin A, Magrabar PGE-42L®, and Clary sage oil.

Another embodiment comprises cyclosporin A, Magrabar PGE-42T®, and Clary sage oil.

Another embodiment comprises cyclosporin A, Magrabar PGE-60-0®, and Clary sage oil.

Another embodiment comprises cyclosporin A, Magrabar PGE-60L®, and Clary sage oil.

Another embodiment comprises cyclosporin A, Magrabar PGE-60T®, and Clary sage oil.

Another embodiment comprises cyclosporin A, Magrabar PGE-62-0®, and Clary sage oil.

Another embodiment comprises cyclosporin A, Magrabar PGE-62L®, and Clary sage oil.

Another embodiment comprises cyclosporin A, Magrabar PGE-62T®, and Clary sage oil.

Another embodiment comprises cyclosporin A, Mapeg S-40K®, and Clary sage oil.

Another embodiment comprises cyclosporin A, Marlowet OTS®, and Clary sage oil.

Another embodiment comprises cyclosporin A, Naturechem PGR®, and Clary sage oil.

Another embodiment comprises cyclosporin A, PG No. 4®, and Clary sage oil.

Another embodiment comprises cyclosporin A, Renex 20®, and Clary sage oil.

Another embodiment comprises cyclosporin A, Ritox 52®, and Clary sage oil.

Another embodiment comprises cyclosporin A, Ritox 53®, and Clary sage oil.

Another embodiment comprises cyclosporin A, Ritox 59®, and Clary sage oil.

Another embodiment comprises cyclosporin A, Surax 8916/A®, and Clary sage oil.

Another embodiment comprises cyclosporin A, Tego Acid S 40 P®, and Clary sage oil.

Another embodiment comprises cyclosporin A, Tego Acid S 100 P®, and Clary sage oil.

Another embodiment comprises cyclosporin A, Tween 20®, and Clary sage oil.

Another embodiment comprises cyclosporin A, Volpo 131®, and Clary sage oil.

Another embodiment comprises cyclosporin A, an Ethoxylated Fatty Ester or Oil, and Clary sage oil.

Another embodiment comprises cyclosporin A, Acconon 6-C10®, and Clary sage oil.

Another embodiment comprises cyclosporin A, Acconon CC-6®, and Clary sage oil.

Another embodiment comprises cyclosporin A, Acconon CO-7®, and Clary sage oil.

Another embodiment comprises cyclosporin A, Aldosperse 40/60 FG®, and Clary sage oil.

Another embodiment comprises cyclosporin A, Aldosperse ML-23®, and Clary sage oil.

Another embodiment comprises cyclosporin A, Aldosperse MS-20 FG®, and Clary sage oil.

Another embodiment comprises cyclosporin A, Alkamuls EL-620®, and Clary sage oil.

Another embodiment comprises cyclosporin A, Alkamuls EL-719®, and Clary sage oil.

Another embodiment comprises cyclosporin A, Alkamuls EL-985®, and Clary sage oil.

Another embodiment comprises cyclosporin A, Arlatone G®, and Clary sage oil.

Another embodiment comprises cyclosporin A, Arlatone T®, and Clary sage oil.

Another embodiment comprises cyclosporin A, Atlas G-1045A®, and Clary sage oil.

Another embodiment comprises cyclosporin A, Atlas G-1086®, and Clary sage oil.

Another embodiment comprises cyclosporin A, Atlas G-1087®, and Clary sage oil.

Another embodiment comprises cyclosporin A, Atlas G-1089®, and Clary sage oil.

Another embodiment comprises cyclosporin A, Atlas G-1096®, and Clary sage oil.

Another embodiment comprises cyclosporin A, Atlas G-1292®, and Clary sage oil.

Another embodiment comprises cyclosporin A, Atlas G-1293®, and Clary sage oil.

Another embodiment comprises cyclosporin A, Atlas G-1300®, and Clary sage oil.

Another embodiment comprises cyclosporin A, Atlas G-7076®, and Clary sage oil.

Another embodiment comprises cyclosporin A, Capmul EMG®, and Clary sage oil.

Another embodiment comprises cyclosporin A, Chemonic CO-40®, and Clary sage oil.

Another embodiment comprises cyclosporin A, Chemonic LI-3®, and Clary sage oil.

Another embodiment comprises cyclosporin A, Chemonic LI-7®, and Clary sage oil.

Another embodiment comprises cyclosporin A, Cirrasol GM®, and Clary sage oil.

Another embodiment comprises cyclosporin A, Cremophor CO 40®, and Clary sage oil.

Another embodiment comprises cyclosporin A, Cremophor CO 410®, and Clary sage oil.

Another embodiment comprises cyclosporin A, Cremophor EL®, and Clary sage oil.

Another embodiment comprises cyclosporin A, Cremophor GC7®, and Clary sage oil.

Another embodiment comprises cyclosporin A, Cremophor RH-40®, and Clary sage oil.

Another embodiment comprises cyclosporin A, Crovol A-40®, and Clary sage oil.

Another embodiment comprises cyclosporin A, Crovol A-70®, and Clary sage oil.

Another embodiment comprises cyclosporin A, Crovol M-70®, and Clary sage oil.

Another embodiment comprises cyclosporin A, Crovol PK-70®, and Clary sage oil.

Another embodiment comprises cyclosporin A, Cutina E-24®, and Clary sage oil.

Another embodiment comprises cyclosporin A, Dacospin 12-R®, and Clary sage oil.

Another embodiment comprises cyclosporin A, Dehymuls HRE-7®, and Clary sage oil.

Another embodiment comprises cyclosporin A, DeSonic 30C®, and Clary sage oil.

Another embodiment comprises cyclosporin A, DeSonic 36C®, and Clary sage oil.

Another embodiment comprises cyclosporin A, DeSonic 40C®, and Clary sage oil.

Another embodiment comprises cyclosporin A, Durfax 60®, and Clary sage oil.

Another embodiment comprises cyclosporin A, Durfax 65®, and Clary sage oil.

Another embodiment comprises cyclosporin A, Durfax 80®, and Clary sage oil.

Another embodiment comprises cyclosporin A, Durfax EOM®, and Clary sage oil.

Another embodiment comprises cyclosporin A, EccotergeNF-2®, and Clary sage oil.

Another embodiment comprises cyclosporin A, Emulpon CC-360®, and Clary sage oil.

Another embodiment comprises cyclosporin A, Emulpon CO-550®, and Clary sage oil.

Another embodiment comprises cyclosporin A, Emulsogen EL®, and Clary sage oil.

Another embodiment comprises cyclosporin A, Emulsogen HCO 040®, and Clary sage oil.

Another embodiment comprises cyclosporin A, Emulsogen HCO 060®, and Clary sage oil.

Another embodiment comprises cyclosporin A, Emulsynt 1055®, and Clary sage oil.

Another embodiment comprises cyclosporin A, Ethox 3095®, and Clary sage oil.

Another embodiment comprises cyclosporin A, Eumulgin RO-40®, and Clary sage oil.

Another embodiment comprises cyclosporin A, Genapol G-260®, and Clary sage oil.

Another embodiment comprises cyclosporin A, Glycosperse L-20®, and Clary sage oil.

Another embodiment comprises cyclosporin A, Glycosperse O-5®, and Clary sage oil.

Another embodiment comprises cyclosporin A, Glycosperse O-20®, and Clary sage oil.

Another embodiment comprises cyclosporin A, Glycosperse O-20 FG®, and Clary sage oil.

Another embodiment comprises cyclosporin A, Glycosperse S-20®, and Clary sage oil.

Another embodiment comprises cyclosporin A, Glycosperse S-20 FG®, and Clary sage oil.

Another embodiment comprises cyclosporin A, Glycosperse TS-20®, and Clary sage oil.

Another embodiment comprises cyclosporin A, Glycosperse TS-20 FG®, and Clary sage oil.

Another embodiment comprises cyclosporin A, Hetan SL®, and Clary sage oil.

Another embodiment comprises cyclosporin A, Hetan SO®, and Clary sage oil.

Another embodiment comprises cyclosporin A, Hetan SS®, and Clary sage oil.

Another embodiment comprises cyclosporin A, Hetoxide C-2®, and Clary sage oil.

Another embodiment comprises cyclosporin A, Hetoxide C-9®, and Clary sage oil.

Another embodiment comprises cyclosporin A, Hetoxide C-15®, and Clary sage oil.

Another embodiment comprises cyclosporin A, Hetoxide C-25®, and Clary sage oil.

Another embodiment comprises cyclosporin A, Hetoxide C-40®, and Clary sage oil.

Another embodiment comprises cyclosporin A, Hetoxide C-200®, and Clary sage oil.

Another embodiment comprises cyclosporin A, Hetoxide C-200-50%®, and Clary sage oil.

Another embodiment comprises cyclosporin A, Hetoxide GC-30®, and Clary sage oil.

Another embodiment comprises cyclosporin A, Hetoxide HC-60®, and Clary sage oil.

Another embodiment comprises cyclosporin A, Ice No. 2®, and Clary sage oil.

Another embodiment comprises cyclosporin A, Incrocas 30/40®, and Clary sage oil.

Another embodiment comprises cyclosporin A, Lexol EC®, and Clary sage oil.

Another embodiment comprises cyclosporin A, Lexol EO®, and Clary sage oil.

Another embodiment comprises cyclosporin A, Lipocol HCO-40®, and Clary sage oil.

Another embodiment comprises cyclosporin A, Lipocol HCO-60®, and Clary sage oil.

Another embodiment comprises cyclosporin A, Lipocol O-3 Special®, and Clary sage oil.

Another embodiment comprises cyclosporin A, Lipopeg 2-L®, and Clary sage oil.

Another embodiment comprises cyclosporin A, Lipopeg 4-DO®, and Clary sage oil.

Another embodiment comprises cyclosporin A, Lipopeg 4-DS®, and Clary sage oil.

Another embodiment comprises cyclosporin A, Lipovol GTB®, and Clary sage oil.

Another embodiment comprises cyclosporin A, Lonzest SML-20®, and Clary sage oil.

Another embodiment comprises cyclosporin A, Lonzest SMO-20®, and Clary sage oil.

Another embodiment comprises cyclosporin A, Lonzest SMS-20®, and Clary sage oil.

Another embodiment comprises cyclosporin A, Lonzest STO-20®, and Clary sage oil.

Another embodiment comprises cyclosporin A, Lonzest STS-20®, and Clary sage oil.

Another embodiment comprises cyclosporin A, Lumulse GR-40®, and Clary sage oil.

Another embodiment comprises cyclosporin A, Lumulse GRH-40®, and Clary sage oil.

Another embodiment comprises cyclosporin A, Lumulse POE (7) GML®, and Clary sage oil.

Another embodiment comprises cyclosporin A, Lumulse POE (12) Glyc®, and Clary sage oil.

Another embodiment comprises cyclosporin A, Lumulse POE (40) MS KP®, and Clary sage oil.

Another embodiment comprises cyclosporin A, Marlowet 4750®, and Clary sage oil.

Another embodiment comprises cyclosporin A, Marlowet LVS®, and Clary sage oil.

Another embodiment comprises cyclosporin A, Marlowet R 11®, and Clary sage oil.

Another embodiment comprises cyclosporin A, Marlowet R 40®, and Clary sage oil.

Another embodiment comprises cyclosporin A, Mazol 80 MGK®, and Clary sage oil.

Another embodiment comprises cyclosporin A, Nonionic Emulsifier T-9®, and Clary sage oil.

Another embodiment comprises cyclosporin A, Oronal LCG®, and Clary sage oil.

Another embodiment comprises cyclosporin A, Polyderm PPI-CO-200®, and Clary sage oil.

Another embodiment comprises cyclosporin A, Polyderm PPI-CO-40®, and Clary sage oil.

Another embodiment comprises cyclosporin A, Rewoderm LI 520-70®, and Clary sage oil.

Another embodiment comprises cyclosporin A, Ritapeg 150 DS®, and Clary sage oil.

Another embodiment comprises cyclosporin A, Softigen 767®, and Clary sage oil.

Another embodiment comprises cyclosporin A, Surfactol 318®, and Clary sage oil.

Another embodiment comprises cyclosporin A, Surfactol 365®, and Clary sage oil.

Another embodiment comprises cyclosporin A, Syn Lube 107®, and Clary sage oil.

Another embodiment comprises cyclosporin A, Syn Lube 728®, and Clary sage oil.

Another embodiment comprises cyclosporin A, Syn Lube 1632H®, and Clary sage oil.

Another embodiment comprises cyclosporin A, Syn Lube 6277-A®, and Clary sage oil.

Another embodiment comprises cyclosporin A, T-Det C-20®, and Clary sage oil.

Another embodiment comprises cyclosporin A, T-Det C-40®, and Clary sage oil.

Another embodiment comprises cyclosporin A, Tally 100 Plus®, and Clary sage oil.

Another embodiment comprises cyclosporin A, Uniperol EL®, and Clary sage oil.

Another embodiment comprises cyclosporin A, a Fatty Ester, and Clary sage oil.

Another embodiment comprises cyclosporin A, Actralube-Syn 147®, and Clary sage oil.

Another embodiment comprises cyclosporin A, Atlas G-1556®, and Clary sage oil.

Another embodiment comprises cyclosporin A, Atlas G-1564®, and Clary sage oil.

Another embodiment comprises cyclosporin A, Atlasol Base Oil S®, and Clary sage oil.

Another embodiment comprises cyclosporin A, Base ML®, and Clary sage oil.

Another embodiment comprises cyclosporin A, Base MT®, and Clary sage oil.

Another embodiment comprises cyclosporin A, Cerasynt 303®, and Clary sage oil.

Another embodiment comprises cyclosporin A, Dermol 1012®, and Clary sage oil.

Another embodiment comprises cyclosporin A, Kemester 4000®, and Clary sage oil.

Another embodiment comprises cyclosporin A, Lactipol S®, and Clary sage oil.

Another embodiment comprises cyclosporin A, Magrabar PGO®, and Clary sage oil.

Another embodiment comprises cyclosporin A, Mayco Base BFO®, and Clary sage oil.

Another embodiment comprises cyclosporin A, Methyl Linoleate®, and Clary sage oil.

Another embodiment comprises cyclosporin A, Pationic 122A®, and Clary sage oil.

Another embodiment comprises cyclosporin A, Pationic 138C®, and Clary sage oil.

Another embodiment comprises cyclosporin A, Pationic CSL®, and Clary sage oil.

Another embodiment comprises cyclosporin A, Pationic ISL®, and Clary sage oil.

Another embodiment comprises cyclosporin A, Pationic SBL®, and Clary sage oil.

Another embodiment comprises cyclosporin A, Pationic SSL®, and Clary sage oil.

Another embodiment comprises cyclosporin A, Ritasol®, and Clary sage oil.

Another embodiment comprises cyclosporin A, Tego Alkanol CS 20®, and Clary sage oil.

Another embodiment comprises cyclosporin A, Tego Alkanol L23 P®, and Clary sage oil.

Another embodiment comprises cyclosporin A, Tego Alkanol S2®, and Clary sage oil.

Another embodiment comprises cyclosporin A, Tego Alkanol S20 P®, and Clary sage oil.

Another embodiment comprises cyclosporin A, Triemulsifier 600 MS®, and Clary sage oil.

Another embodiment comprises cyclosporin A, a Glycerol Esters, and Clary sage oil.

Another embodiment comprises cyclosporin A, Agro #9 Wint SBO®, and Clary sage oil.

Another embodiment comprises cyclosporin A, Ahcovel Base 700®, and Clary sage oil.

Another embodiment comprises cyclosporin A, Aldo HMS FG®, and Clary sage oil.

Another embodiment comprises cyclosporin A, Aldo MLD®,and Clary sage oil.

Another embodiment comprises cyclosporin A, Aldo MLD FG®, and Clary sage oil.

Another embodiment comprises cyclosporin A, Aldo MO FG®, and Clary sage oil.

Another embodiment comprises cyclosporin A, Aldo MS®, and Clary sage oil.

Another embodiment comprises cyclosporin A, Aldo MS FG®, and Clary sage oil.

Another embodiment comprises cyclosporin A, Aldo MS LG FG®, and Clary sage oil.

Another embodiment comprises cyclosporin A, Aldo MSD®, and Clary sage oil.

Another embodiment comprises cyclosporin A, Aldo MSD FG®, and Clary sage oil.

Another embodiment comprises cyclosporin A, Aldosperse O-20 FG®, and Clary sage oil.

Another embodiment comprises cyclosporin A, Aldosperse TS-20 FG®, and Clary sage oil.

Another embodiment comprises cyclosporin A, Aldosperse TS-40 FG®, and Clary sage oil.

Another embodiment comprises cyclosporin A, Arlacel 165®, and Clary sage oil.

Another embodiment comprises cyclosporin A, Arlacel 186®, and Clary sage oil.

Another embodiment comprises cyclosporin A, Capmul GMO®, and Clary sage oil.

Another embodiment comprises cyclosporin A, Capmul GMS®, and Clary sage oil.

Another embodiment comprises cyclosporin A, Caprol 3GO®, and Clary sage oil.

Another embodiment comprises cyclosporin A, Caprol 3GVS®, and Clary sage oil.

Another embodiment comprises cyclosporin A, Caprol 6G2S®, and Clary sage oil.

Another embodiment comprises cyclosporin A, Caprol 10G40®, and Clary sage oil.

Another embodiment comprises cyclosporin A, Caprol 10G100®, and Clary sage oil.

Another embodiment comprises cyclosporin A, Caprol ET®, and Clary sage oil.

Another embodiment comprises cyclosporin A, Caprol PGE860®, and Clary sage oil.

Another embodiment comprises cyclosporin A, Cerasynt 945®, and Clary sage oil.

Another embodiment comprises cyclosporin A, Cerasynt GMS®, and Clary sage oil.

Another embodiment comprises cyclosporin A, Cerasynt Q®, and Clary sage oil.

Another embodiment comprises cyclosporin A, Cerasynt SD®, and Clary sage oil.

Another embodiment comprises cyclosporin A, Cerasynt WM®, and Clary sage oil.

Another embodiment comprises cyclosporin A, Chemsperse 14®, and Clary sage oil.

Another embodiment comprises cyclosporin A, Cremophor GO-32®, and Clary sage oil.

Another embodiment comprises cyclosporin A, Cremophor GS11®, and Clary sage oil.

Another embodiment comprises cyclosporin A, Cremophor GS-32®, and Clary sage oil.

Another embodiment comprises cyclosporin A, Cutina KD-16®, and Clary sage oil.

Another embodiment comprises cyclosporin A, Dehymuls PGPH®, and Clary sage oil.

Another embodiment comprises cyclosporin A, Dermol DGDIS®, and Clary sage oil.

Another embodiment comprises cyclosporin A, Dermol DGMIS®, and Clary sage oil.

Another embodiment comprises cyclosporin A, Dermol G-76®, and Clary sage oil.

Another embodiment comprises cyclosporin A, Dermol G-7DI®, and Clary sage oil.

Another embodiment comprises cyclosporin A, Dermol NGDI®, and Clary sage oil.

Another embodiment comprises cyclosporin A, Dermolan GLH®, and Clary sage oil.

Another embodiment comprises cyclosporin A, Drewmulse GMO®, and Clary sage oil.

Another embodiment comprises cyclosporin A, Drewpol 3-5-M®, and Clary sage oil.

Another embodiment comprises cyclosporin A, Durlac 100 W®, and Clary sage oil.

Another embodiment comprises cyclosporin A, Dur-Lo®, and Clary sage oil.

Another embodiment comprises cyclosporin A, Dynasan 118®, and Clary sage oil.

Another embodiment comprises cyclosporin A, EC-25®, and Clary sage oil.

Another embodiment comprises cyclosporin A, EM 40®, and Clary sage oil.

Another embodiment comprises cyclosporin A, Emerest 2400®, and Clary sage oil.

Another embodiment comprises cyclosporin A, Emerest 2452®, and Clary sage oil.

Another embodiment comprises cyclosporin A, Empilan G-26®, and Clary sage oil.

Another embodiment comprises cyclosporin A, Genapol TSM®, and Clary sage oil.

Another embodiment comprises cyclosporin A, Hostacerin DGI®, and Clary sage oil.

Another embodiment comprises cyclosporin A, Hostacerin DGL®, and Clary sage oil.

Another embodiment comprises cyclosporin A, Hostacerin DGMS®, and Clary sage oil.

Another embodiment comprises cyclosporin A, Hostacerin DGSB®, and Clary sage oil.

Another embodiment comprises cyclosporin A, Ice No. 2®, and Clary sage oil.

Another embodiment comprises cyclosporin A, Imwitor 742®, and Clary sage oil.

Another embodiment comprises cyclosporin A, Imwitor 780 K®, and Clary sage oil.

Another embodiment comprises cyclosporin A, Imwitor 960 Flakes®, and Clary sage oil.

Another embodiment comprises cyclosporin A, Isolan GI 34®, and Clary sage oil.

Another embodiment comprises cyclosporin A, Isolan GO 33®, and Clary sage oil.

Another embodiment comprises cyclosporin A, Kemester 1000®, and Clary sage oil.

Another embodiment comprises cyclosporin A, Kemester 2000®, and Clary sage oil.

Another embodiment comprises cyclosporin A, Kemester 2000®, and Clary sage oil.

Another embodiment comprises cyclosporin A, Kemester 6000SE®, and Clary sage oil.

Another embodiment comprises cyclosporin A, Lamecreme DGE 18®, and Clary sage oil.

Another embodiment comprises cyclosporin A, Lexemul 515®, and Clary sage oil.

Another embodiment comprises cyclosporin A, Lexemul 561®, and Clary sage oil.

Another embodiment comprises cyclosporin A, Lexemul AR®, and Clary sage oil.

Another embodiment comprises cyclosporin A, Lexemul AS®, and Clary sage oil.

Another embodiment comprises cyclosporin A, Lexemul GDL®, and Clary sage oil.

Another embodiment comprises cyclosporin A, Lexemul T®, and Clary sage oil.

Another embodiment comprises cyclosporin A, Lipomulse 165®, and Clary sage oil.

Another embodiment comprises cyclosporin A, Lumulse GML K®, and Clary sage oil.

Another embodiment comprises cyclosporin A, Lumulse GMO K®, and Clary sage oil.

Another embodiment comprises cyclosporin A, Lumulse GMR K®, and Clary sage oil.

Another embodiment comprises cyclosporin A, Lumulse GMT K®, and Clary sage oil.

Another embodiment comprises cyclosporin A, Magrabar GMC®, and Clary sage oil.

Another embodiment comprises cyclosporin A, Magrabar GMO-CK®, and Clary sage oil.

Another embodiment comprises cyclosporin A, Magrabar MDG-5050®, and Clary sage oil.

Another embodiment comprises cyclosporin A, Magrabar PGO-315®, and Clary sage oil.

Another embodiment comprises cyclosporin A, Magrabar PGO-1010®, and Clary sage oil.

Another embodiment comprises cyclosporin A, Mazol 300K®, and Clary sage oil.

Another embodiment comprises cyclosporin A, Mazol GMO-K®, and Clary sage oil.

Another embodiment comprises cyclosporin A, Mazol GMS-K®, and Clary sage oil.

Another embodiment comprises cyclosporin A, Mazol PG031-K®, and Clary sage oil.

Another embodiment comprises cyclosporin A, Miglyol 812®, and Clary sage oil.

Another embodiment comprises cyclosporin A, Norfox 165C®, and Clary sage oil.

Another embodiment comprises cyclosporin A, Schercemol GMIS®, and Clary sage oil.

Another embodiment comprises cyclosporin A, Tegin®, and Clary sage oil.

Another embodiment comprises cyclosporin A, Tegin 4100 Pellets®, and Clary sage oil.

Another embodiment comprises cyclosporin A, Tegin M Pellets®, and Clary sage oil.

Another embodiment comprises cyclosporin A, Tegin OV®, and Clary sage oil.

Another embodiment comprises cyclosporin A, Teginacid H®, and Clary sage oil.

Another embodiment comprises cyclosporin A, Tego Cosmo P813®, and Clary sage oil.

Another embodiment comprises cyclosporin A, Wickenol 535®, and Clary sage oil.

Another embodiment comprises cyclosporin A, Witconol 14®, and Clary sage oil.

Another embodiment comprises cyclosporin A, Witconol 14®, and Clary sage oil.

Another embodiment comprises cyclosporin A, Witconol 14F®, and Clary sage oil.

Another embodiment comprises cyclosporin A, Witconol 18L®, and Clary sage oil.

Another embodiment comprises cyclosporin A, Witconol GOT®, and Clary sage oil.

Another embodiment comprises cyclosporin A, Witconol MST®, and Clary sage oil.

Another embodiment comprises cyclosporin A, Witconol RHT®, and Clary sage oil.

Another embodiment comprises cyclosporin A, a Glycol Ester, and Clary sage oil.

Another embodiment comprises cyclosporin A, Alkamuls 600 DO®, and Clary sage oil.

Another embodiment comprises cyclosporin A, Alkamuls SEG®, and Clary sage oil.

Another embodiment comprises cyclosporin A, Atlas EM-2®, and Clary sage oil.

Another embodiment comprises cyclosporin A, Cerasynt IP®, and Clary sage oil.

Another embodiment comprises cyclosporin A, Cerasynt M®, and Clary sage oil.

Another embodiment comprises cyclosporin A, Cerasynt MN®, and Clary sage oil.

Another embodiment comprises cyclosporin A, Cerasynt PA®, and Clary sage oil.

Another embodiment comprises cyclosporin A, Chemsperse EGDS®, and Clary sage oil.

Another embodiment comprises cyclosporin A, Chemsperse EGMS®, and Clary sage oil.

Another embodiment comprises cyclosporin A, Colonial Monolaurin®, and Clary sage oil.

Another embodiment comprises cyclosporin A, DeMuls SGE-95®, and Clary sage oil.

Another embodiment comprises cyclosporin A, Eccoterge 200®, and Clary sage oil.

Another embodiment comprises cyclosporin A, Emerest 2380®, and Clary sage oil.

Another embodiment comprises cyclosporin A, Ethox 2610®, and Clary sage oil.

Another embodiment comprises cyclosporin A, Ethox DO-9®, and Clary sage oil.

Another embodiment comprises cyclosporin A, Ethox DO-14®, and Clary sage oil.

Another embodiment comprises cyclosporin A, Ethox SO-9®, and Clary sage oil.

Another embodiment comprises cyclosporin A, Fizul MD-318®, and Clary sage oil.

Another embodiment comprises cyclosporin A, Genapol EGDS-VHP®, and Clary sage oil.

Another embodiment comprises cyclosporin A, Genapol TS Powder®, and Clary sage oil.

Another embodiment comprises cyclosporin A, Hostacerin WO®, and Clary sage oil.

Another embodiment comprises cyclosporin A, Inversol 140®, and Clary sage oil.

Another embodiment comprises cyclosporin A, Kemester 104®, and Clary sage oil.

Another embodiment comprises cyclosporin A, Kemester 205®, and Clary sage oil.

Another embodiment comprises cyclosporin A, Kemester 226®, and Clary sage oil.

Another embodiment comprises cyclosporin A, Kemester 5221SE®, and Clary sage oil.

Another embodiment comprises cyclosporin A, Kemester EGDS®, and Clary sage oil.

Another embodiment comprises cyclosporin A, Lexemul EGDS®, and Clary sage oil.

Another embodiment comprises cyclosporin A, Lexemul EGMS®, and Clary sage oil.

Another embodiment comprises cyclosporin A, Lexemul P®, and Clary sage oil.

Another embodiment comprises cyclosporin A, Lipo DGLS®, and Clary sage oil.

Another embodiment comprises cyclosporin A, Lipo EGDS®, and Clary sage oil.

Another embodiment comprises cyclosporin A, Lipo PGMS®, and Clary sage oil.

Another embodiment comprises cyclosporin A, Liposorb S-4®, and Clary sage oil.

Another embodiment comprises cyclosporin A, Liposorb TO-20®, and Clary sage oil.

Another embodiment comprises cyclosporin A, Lumulse PGO®, and Clary sage oil.

Another embodiment comprises cyclosporin A, Mackester EGDS®, and Clary sage oil.

Another embodiment comprises cyclosporin A, Mackester EGMS®, and Clary sage oil.

Another embodiment comprises cyclosporin A, Mackester GSTP®, and Clary sage oil.

Another embodiment comprises cyclosporin A, Mackester Series®, and Clary sage oil.

Another embodiment comprises cyclosporin A, Magrabar PDG-50®, and Clary sage oil.

Another embodiment comprises cyclosporin A, Mapeg 6000 DS®, and Clary sage oil.

Another embodiment comprises cyclosporin A, Marlowet 4702®, and Clary sage oil.

Another embodiment comprises cyclosporin A, Monalube 305®, and Clary sage oil.

Another embodiment comprises cyclosporin A, Monalube 310®, and Clary sage oil.

Another embodiment comprises cyclosporin A, Monalube 315®, and Clary sage oil.

Another embodiment comprises cyclosporin A, Monalube 320®, and Clary sage oil.

Another embodiment comprises cyclosporin A, Monalube 325®, and Clary sage oil.

Another embodiment comprises cyclosporin A, Monalube 330®, and Clary sage oil.

Another embodiment comprises cyclosporin A, Naturechem PGHS®, and Clary sage oil.

Another embodiment comprises cyclosporin A, Polycastorol PLO-840®, and Clary sage oil.

Another embodiment comprises cyclosporin A, Polytex 10M®, and Clary sage oil.

Another embodiment comprises cyclosporin A, Ritasynt IP®, and Clary sage oil.

Another embodiment comprises cyclosporin A, Ross Chem PEG 600 DT®, and Clary sage oil.

Another embodiment comprises cyclosporin A, Schercemol PGMS®, and Clary sage oil.

Another embodiment comprises cyclosporin A, Sponto H-44C®, and Clary sage oil.

Another embodiment comprises cyclosporin A, Tegin G®, and Clary sage oil.

Another embodiment comprises cyclosporin A, Witbreak DGE-182®, and Clary sage oil.

Another embodiment comprises cyclosporin A, Witbreak DGE-182®, and Clary sage oil.

Another embodiment comprises cyclosporin A, Witbreak DRA-21®, and Clary sage oil.

Another embodiment comprises cyclosporin A, Witbreak DRA-21®, and Clary sage oil.

Another embodiment comprises cyclosporin A, Witbreak DRA-50®, and Clary sage oil.

Another embodiment comprises cyclosporin A, Witbreak DRA-50®, and Clary sage oil.

Another embodiment comprises cyclosporin A, Witconol F26-46®, and Clary sage oil.

Another embodiment comprises cyclosporin A, Witconol H-32®, and Clary sage oil.

Another embodiment comprises cyclosporin A, Witconol H-33®, and Clary sage oil.

Another embodiment comprises cyclosporin A, Witconol H-35A®, and Clary sage oil.

Another embodiment comprises cyclosporin A, Witconol RHP®, and Clary sage oil.

Another embodiment comprises cyclosporin A, a Lanolin-based Derivative, and Clary sage oil.

Another embodiment comprises cyclosporin A, Amerchol CAB®, and Clary sage oil.

Another embodiment comprises cyclosporin A, Amerchol L-101®, and Clary sage oil.

Another embodiment comprises cyclosporin A, Amerlate LFA-LO®, and Clary sage oil.

Another embodiment comprises cyclosporin A, Amerlate P®, and Clary sage oil.

Another embodiment comprises cyclosporin A, Barre Common Degras®, and Clary sage oil.

Another embodiment comprises cyclosporin A, Cholesterol NF®, and Clary sage oil.

Another embodiment comprises cyclosporin A, Crodalan AWS®, and Clary sage oil.

Another embodiment comprises cyclosporin A, Crodalan LA®, and Clary sage oil.

Another embodiment comprises cyclosporin A, Emery 1650®, and Clary sage oil.

Another embodiment comprises cyclosporin A, Emery 1650®, and Clary sage oil.

Another embodiment comprises cyclosporin A, Emery 1740®, and Clary sage oil.

Another embodiment comprises cyclosporin A, Emery 1740®, and Clary sage oil.

Another embodiment comprises cyclosporin A, Forlan 500®, and Clary sage oil.

Another embodiment comprises cyclosporin A, Forlan L®, and Clary sage oil.

Another embodiment comprises cyclosporin A, Laneto 50®, and Clary sage oil.

Another embodiment comprises cyclosporin A, Laneto 100®, and Clary sage oil.

Another embodiment comprises cyclosporin A, Laneto AWS®, and Clary sage oil.

Another embodiment comprises cyclosporin A, Lanfrax 1776®, and Clary sage oil.

Another embodiment comprises cyclosporin A, Lanfrax 1776®, and Clary sage oil.

Another embodiment comprises cyclosporin A, Lanogel 21®, and Clary sage oil.

Another embodiment comprises cyclosporin A, Lipolan®, and Clary sage oil.

Another embodiment comprises cyclosporin A, Lipolan 31®, and Clary sage oil.

Another embodiment comprises cyclosporin A, OHlan®, and Clary sage oil.

Another embodiment comprises cyclosporin A, Polychol 5®, and Clary sage oil.

Another embodiment comprises cyclosporin A, Polychol 15®, and Clary sage oil.

Another embodiment comprises cyclosporin A, Ritacetyl®, and Clary sage oil.

Another embodiment comprises cyclosporin A, Ritachol®, and Clary sage oil.

Another embodiment comprises cyclosporin A, Ritahydrox®, and Clary sage oil.

Another embodiment comprises cyclosporin A: Ritalafa®, and Clary sage oil.

Another embodiment comprises cyclosporin A, Ritalan®, and Clary sage oil.

Another embodiment comprises cyclosporin A, Ritalan AWS®, and Clary sage oil.

Another embodiment comprises cyclosporin A, Ritalan C®, and Clary sage oil.

Another embodiment comprises cyclosporin A, Ritawax®, and Clary sage oil.

Another embodiment comprises cyclosporin A, Ritawax AEO®, and Clary sage oil.

Another embodiment comprises cyclosporin A, Ritawax ALA®, and Clary sage oil.

Another embodiment comprises cyclosporin A, Solan/Solan 50/Super Solan®, and Clary sage oil.

Another embodiment comprises cyclosporin A, Super Hartolan/Hartolan®, and Clary sage oil.

Another embodiment comprises cyclosporin A, Supersat AWS-4®, and Clary sage oil.

Another embodiment comprises cyclosporin A, Supersat AWS-24®, and Clary sage oil.

Another embodiment comprises cyclosporin A, a Lecithin or Lecithin Derivative, and Clary sage oil.

Another embodiment comprises cyclosporin A, Alcolec®, and Clary sage oil.

Another embodiment comprises cyclosporin A, Lecithin®, and Clary sage oil.

Another embodiment comprises cyclosporin A, Lexin K®, and Clary sage oil.

Another embodiment comprises cyclosporin A, Natipide®, and Clary sage oil.

Another embodiment comprises cyclosporin A, a Lignin or Lignin Derivative, and Clary sage oil.

Another embodiment comprises cyclosporin A, Diwatex XP 9®, and Clary sage oil.

Another embodiment comprises cyclosporin A, Dynasperse LCD®, and Clary sage oil.

Another embodiment comprises cyclosporin A, Indulin SAL®, and Clary sage oil.

Another embodiment comprises cyclosporin A, Indulin W-1®, and Clary sage oil.

Another embodiment comprises cyclosporin A, Indulin W-5®, and Clary sage oil.

Another embodiment comprises cyclosporin A, Lignosol FTA®, and Clary sage oil.

Another embodiment comprises cyclosporin A, Lignosol SFX-65®, and Clary sage oil.

Another embodiment comprises cyclosporin A, Marasperse 52 CP®, and Clary sage oil.

Another embodiment comprises cyclosporin A, Marasperse AG®, and Clary sage oil.

Another embodiment comprises cyclosporin A, Marasperse CBOS-4®, and Clary sage oil.

Another embodiment comprises cyclosporin A, Ufoxane 2®, and Clary sage oil.

Another embodiment comprises cyclosporin A, a Methyl Ester, and Clary sage oil.

Another embodiment comprises cyclosporin A, E.B. Cleaner AK®, and Clary sage oil.

Another embodiment comprises cyclosporin A, Oleocal ME-70®, and Clary sage oil.

Another embodiment comprises cyclosporin A, Trade Name Company®, and Clary sage oil.

Another embodiment comprises cyclosporin A, Methyl Esters (cont'd)®, and Clary sage oil.

Another embodiment comprises cyclosporin A, Oleocal ME-92®, and Clary sage oil.

Another embodiment comprises cyclosporin A, Oleocal ME-112®, and Clary sage oil.

Another embodiment comprises cyclosporin A, Oleocal ME-130®, and Clary sage oil.

Another embodiment comprises cyclosporin A, a Monoglyceride or a Derivative thereof, and Clary sage oil.

Another embodiment comprises cyclosporin A, Dynacet 211®, and Clary sage oil.

Another embodiment comprises cyclosporin A, Hetsorb S-20®, and Clary sage oil.

Another embodiment comprises cyclosporin A, Imwitor 191®, and Clary sage oil.

Another embodiment comprises cyclosporin A, Imwitor 370®, and Clary sage oil.

Another embodiment comprises cyclosporin A, Imwitor 375®, and Clary sage oil.

Another embodiment comprises cyclosporin A, Imwitor 900®, and Clary sage oil.

Another embodiment comprises cyclosporin A, Imwitor 945®, and Clary sage oil.

Another embodiment comprises cyclosporin A, Imwitor 2020®, and Clary sage oil.

Another embodiment comprises cyclosporin A, Kemester 5500®, and Clary sage oil.

Another embodiment comprises cyclosporin A, Kemester 6000®, and Clary sage oil.

Another embodiment comprises cyclosporin A, Magrabar GMC®, and Clary sage oil.

Another embodiment comprises cyclosporin A, Magrabar GMO-CK®, and Clary sage oil.

Another embodiment comprises cyclosporin A, Magrabar GPC-10®, and Clary sage oil.

Another embodiment comprises cyclosporin A, Magrabar MDG-5050®, and Clary sage oil.

Another embodiment comprises cyclosporin A, Monalube 335®, and Clary sage oil.

Another embodiment comprises cyclosporin A, Monoglycerides Glyceryl Monestearate Archer Daniels Midland Company®, and Clary sage oil.

Another embodiment comprises cyclosporin A, Rita GMS®, and Clary sage oil.

Another embodiment comprises cyclosporin A, Ritamulse SCG®, and Clary sage oil.

Another embodiment comprises cyclosporin A, Softigen 701®, and Clary sage oil.

Another embodiment comprises cyclosporin A, Tally 100 Plus®, and Clary sage oil.

Another embodiment comprises cyclosporin A, a Polyethylene Glycol, and Clary sage oil.

Another embodiment comprises cyclosporin A, Emulgade PL 68/50®, and Clary sage oil.

Another embodiment comprises cyclosporin A, Lumulse PEG®, and Clary sage oil.

Another embodiment comprises cyclosporin A, Rhodasurf PEG-400®, and Clary sage oil.

Another embodiment comprises cyclosporin A, Rhodasun PEG-600®, and Clary sage oil.

Another embodiment comprises cyclosporin A, Witconol PEG-400®, and Clary sage oil.

Another embodiment comprises cyclosporin A, a Polymeric Surfactant, and Clary sage oil.

Another embodiment comprises cyclosporin A, Acritamer PNC-EG®, and Clary sage oil.

Another embodiment comprises cyclosporin A, Ag-Rho DEP-775®, and Clary sage oil.

Another embodiment comprises cyclosporin A, APG 325N Glycoside®, and Clary sage oil.

Another embodiment comprises cyclosporin A, Aristoflex AVC®, and Clary sage oil.

Another embodiment comprises cyclosporin A, Aristoflex HMB®, and Clary sage oil.

Another embodiment comprises cyclosporin A, Burco NPS-225®, and Clary sage oil.

Another embodiment comprises cyclosporin A, Burco NPS-816®, and Clary sage oil.

Another embodiment comprises cyclosporin A, Chemccinate 5603®, and Clary sage oil.

Another embodiment comprises cyclosporin A, Cosmedia Guar C-261N®, and Clary sage oil.

Another embodiment comprises cyclosporin A, Gantrez S-95®, and Clary sage oil.

Another embodiment comprises cyclosporin A, Glucopon 220 UP®, and Clary sage oil.

Another embodiment comprises cyclosporin A, Glucopon 225 DK®, and Clary sage oil.

Another embodiment comprises cyclosporin A, Glucopon 425 N®, and Clary sage oil.

Another embodiment comprises cyclosporin A, Glucopon 600 UP®, and Clary sage oil.

Another embodiment comprises cyclosporin A, Glucopon 625 UP®, and Clary sage oil.

Another embodiment comprises cyclosporin A, Pemulen 1621®, and Clary sage oil.

Another embodiment comprises cyclosporin A, Pemulen 1622®, and Clary sage oil.

Another embodiment comprises cyclosporin A, Pemulen TR-1®, and Clary sage oil.

Another embodiment comprises cyclosporin A, Pemulen TR-2®, and Clary sage oil.

Another embodiment comprises cyclosporin A, Plantacare 818®, and Clary sage oil.

Another embodiment comprises cyclosporin A, Plantapon LGC Sorb®, and Clary sage oil.

Another embodiment comprises cyclosporin A, Plantaren 1200N®, and Clary sage oil.

Another embodiment comprises cyclosporin A, Plantaren 2000N®, and Clary sage oil.

Another embodiment comprises cyclosporin A, Viscolam AT 64®, and Clary sage oil.

Another embodiment comprises cyclosporin A, Viscolam AT 64P®, and Clary sage oil.

Another embodiment comprises cyclosporin A, Viscolam AT 100®, and Clary sage oil.

Another embodiment comprises cyclosporin A, Viscolam MAC 7®, and Clary sage oil.

Another embodiment comprises cyclosporin A, Viscolam SMC 20®, and Clary sage oil.

Another embodiment comprises cyclosporin A, Witbreak RTC-323®, and Clary sage oil.

Another embodiment comprises cyclosporin A, WSI 3700®, and Clary sage oil.

Another embodiment comprises cyclosporin A, a Propoxylated or Ethoxylated Fatty Acid, Alcohol, or Alkyl Phenol, and Clary sage oil.

Another embodiment comprises cyclosporin A, Antarox AA-60®, and Clary sage oil.

Another embodiment comprises cyclosporin A, Antarox LF-224®, and Clary sage oil.

Another embodiment comprises cyclosporin A, Burcomul DFE-45®, and Clary sage oil.

Another embodiment comprises cyclosporin A, Burcoterge LFE-1000®, and Clary sage oil.

Another embodiment comprises cyclosporin A, Chemal LF-25B®, and Clary sage oil.

Another embodiment comprises cyclosporin A, Chemal LF-40B®, and Clary sage oil.

Another embodiment comprises cyclosporin A, Dehypon LS-36®, and Clary sage oil.

Another embodiment comprises cyclosporin A, Dehypon LS-36®, and Clary sage oil.

Another embodiment comprises cyclosporin A, Dehypon LS-54®, and Clary sage oil.

Another embodiment comprises cyclosporin A, Dehypon LS-54®, and Clary sage oil.

Another embodiment comprises cyclosporin A, Delonic 100 VLF®, and Clary sage oil.

Another embodiment comprises cyclosporin A, Delonic LF-60 MOD®, and Clary sage oil.

Another embodiment comprises cyclosporin A, Empiderm B®, and Clary sage oil.

Another embodiment comprises cyclosporin A, Ethylan 1206®, and Clary sage oil.

Another embodiment comprises cyclosporin A, Ethylan NS-500K®, and Clary sage oil.

Another embodiment comprises cyclosporin A, Ethylan NS-500LQ®, and Clary sage oil.

Another embodiment comprises cyclosporin A, Genapol 1392®, and Clary sage oil.

Another embodiment comprises cyclosporin A, Genapol 2317®, and Clary sage oil.

Another embodiment comprises cyclosporin A, Genapol 26EP710®, and Clary sage oil.

Another embodiment comprises cyclosporin A, Genapol EP 1022®, and Clary sage oil.

Another embodiment comprises cyclosporin A, Genapol EP 1024®, and Clary sage oil.

Another embodiment comprises cyclosporin A, Genapol EP 6068®, and Clary sage oil.

Another embodiment comprises cyclosporin A, Genapol NP915®, and Clary sage oil.

Another embodiment comprises cyclosporin A, Kieralon MFB®, and Clary sage oil.

Another embodiment comprises cyclosporin A, Lumisolve CSA-80 V®, and Clary sage oil.

Another embodiment comprises cyclosporin A, Marlowet 5001®, and Clary sage oil.

Another embodiment comprises cyclosporin A, Marlox FK 64®, and Clary sage oil.

Another embodiment comprises cyclosporin A, Marlox MO 124®, and Clary sage oil.

Another embodiment comprises cyclosporin A, Marlox S 58®, and Clary sage oil.

Another embodiment comprises cyclosporin A, Nonatell 1003®, and Clary sage oil.

Another embodiment comprises cyclosporin A, Nonatell 1038®, and Clary sage oil.

Another embodiment comprises cyclosporin A, Nonatell 1052®, and Clary sage oil.

Another embodiment comprises cyclosporin A, Nonatell 1061®, and Clary sage oil.

Another embodiment comprises cyclosporin A, Nonatell 1075®, and Clary sage oil.

Another embodiment comprises cyclosporin A, Nonatell 1088®, and Clary sage oil.

Another embodiment comprises cyclosporin A, Nonatell 1123®, and Clary sage oil.

Another embodiment comprises cyclosporin A, Nonatell 1153®, and Clary sage oil.

Another embodiment comprises cyclosporin A, Nonatell 1161®, and Clary sage oil.

Another embodiment comprises cyclosporin A, Nonatell 1172®, and Clary sage oil.

Another embodiment comprises cyclosporin A, Nonatell 1181®, and Clary sage oil.

Another embodiment comprises cyclosporin A, Norfox 36®, and Clary sage oil.

Another embodiment comprises cyclosporin A, Procetyl AWS®, and Clary sage oil.

Another embodiment comprises cyclosporin A, Sandoxylate SX 412®, and Clary sage oil.

Another embodiment comprises cyclosporin A, Sandoxylate SX 418®, and Clary sage oil.

Another embodiment comprises cyclosporin A, Surfonic JL-80X®, and Clary sage oil.

Another embodiment comprises cyclosporin A, Surfonic JL-80X-B1®, and Clary sage oil.

Another embodiment comprises cyclosporin A, Surfonic L4-29X®, and Clary sage oil.

Another embodiment comprises cyclosporin A, Sunonic LF®, and Clary sage oil.

Another embodiment comprises cyclosporin A, T-Det A826®, and Clary sage oil.

Another embodiment comprises cyclosporin A, T-Det LF-416®, and Clary sage oil.

Another embodiment comprises cyclosporin A, Tergitol Min-Foam 1X®, and Clary sage oil.

Another embodiment comprises cyclosporin A, Tergitol Min-Foam 2X®, and Clary sage oil.

Another embodiment comprises cyclosporin A, Triton CF-21®, and Clary sage oil.

Another embodiment comprises cyclosporin A, Triton CF-76®, and Clary sage oil.

Another embodiment comprises cyclosporin A, Triton XL-80N®, and Clary sage oil.

Another embodiment comprises cyclosporin A, Witconol NS-98®, and Clary sage oil.

Another embodiment comprises cyclosporin A, Witconol NS-108LQ®, and Clary sage oil.

Another embodiment comprises cyclosporin A, Witconol NS-145®, and Clary sage oil.

Another embodiment comprises cyclosporin A, Witconol NS-179®, and Clary sage oil.

Another embodiment comprises cyclosporin A, a Protein-based Surfactant, and Clary sage oil.

Another embodiment comprises cyclosporin A, AminoFoam W®, and Clary sage oil.

Another embodiment comprises cyclosporin A, Amiter LGOD-2®, and Clary sage oil.

Another embodiment comprises cyclosporin A, Amiter LGS-2®, and Clary sage oil.

Another embodiment comprises cyclosporin A, Amiter LGS-5®, and Clary sage oil.

Another embodiment comprises cyclosporin A, Lamepon S®, and Clary sage oil.

Another embodiment comprises cyclosporin A, Lamepon S®, and Clary sage oil.

Another embodiment comprises cyclosporin A, Maypon 4C®, and Clary sage oil.

Another embodiment comprises cyclosporin A, May-Tein C®, and Clary sage oil.

Another embodiment comprises cyclosporin A, May-Tein CT®, and Clary sage oil.

Another embodiment comprises cyclosporin A, May-Tein KTS®, and Clary sage oil.

Another embodiment comprises cyclosporin A, May-Tein SY®, and Clary sage oil.

Another embodiment comprises cyclosporin A, Plantapon S®, and Clary sage oil.

Another embodiment comprises cyclosporin A, Proteol APL®, and Clary sage oil.

Another embodiment comprises cyclosporin A, Proteol OAT®, and Clary sage oil.

Another embodiment comprises cyclosporin A, Pyroter CPI-40®, and Clary sage oil.

Another embodiment comprises cyclosporin A, Pyroter GPI-25®, and Clary sage oil.

Another embodiment comprises cyclosporin A, Supro-Tein S®, and Clary sage oil.

Another embodiment comprises cyclosporin A, Supro-Tein V®, and Clary sage oil.

Another embodiment comprises cyclosporin A,® and Clary sage oil.

Another embodiment comprises cyclosporin A, a Sarcosine Derivative®, and Clary sage oil.

Another embodiment comprises cyclosporin A, Crodasinic LS-30®, and Clary sage oil.

Another embodiment comprises cyclosporin A, Vanseal CS®, and Clary sage oil.

Another embodiment comprises cyclosporin A, Vanseal LS®, and Clary sage oil.

Another embodiment comprises cyclosporin A, Vanseal MS®, and Clary sage oil.

Another embodiment comprises cyclosporin A, Vanseal NACS-30®, and Clary sage oil.

Another embodiment comprises cyclosporin A, Vanseal NALS-95®, and Clary sage oil.

Another embodiment comprises cyclosporin A, Vanseal OS®, and Clary sage oil.

Another embodiment comprises cyclosporin A,® and Clary sage oil.

Another embodiment comprises cyclosporin A, a Silicone-based Surfactant®, and Clary sage oil.

Another embodiment comprises cyclosporin A, Abil-B-9950®, and Clary sage oil.

Another embodiment comprises cyclosporin A, Abil Care 85®, and Clary sage oil.

Another embodiment comprises cyclosporin A, Abil EM 90®, and Clary sage oil.

Another embodiment comprises cyclosporin A, Abil EM 97®, and Clary sage oil.

Another embodiment comprises cyclosporin A, Abil WE-09®, and Clary sage oil.

Another embodiment comprises cyclosporin A, Dow Corning 1248 Fluid®, and Clary sage oil.

Another embodiment comprises cyclosporin A, Dow Corning 3225C®, and Clary sage oil.

Another embodiment comprises cyclosporin A, Dow Corning 5200®, and Clary sage oil.

Another embodiment comprises cyclosporin A, Dow Corning Q4-3667®, and Clary sage oil.

Another embodiment comprises cyclosporin A, Monasil PCA®, and Clary sage oil.

Another embodiment comprises cyclosporin A, Monasil PDM®, and Clary sage oil.

Another embodiment comprises cyclosporin A, Monasil PLN®, and Clary sage oil.

Another embodiment comprises cyclosporin A, Polyderm PPI-SI-WS®, and Clary sage oil.

Another embodiment comprises cyclosporin A, Troysol 380W®, and Clary sage oil.

Another embodiment comprises cyclosporin A, Troysol S366®, and Clary sage oil.

Another embodiment comprises cyclosporin A, a Sorbitan Derivative, and Clary sage oil.

Another embodiment comprises cyclosporin A, Alkamuls SML®, and Clary sage oil.

Another embodiment comprises cyclosporin A, Alkamuls SMO®, and Clary sage oil.

Another embodiment comprises cyclosporin A, Alkamuls STO®, and Clary sage oil.

Another embodiment comprises cyclosporin A, Arlacel 20®, and Clary sage oil.

Another embodiment comprises cyclosporin A, Arlacel 40®, and Clary sage oil.

Another embodiment comprises cyclosporin A, Arlacel 60®, and Clary sage oil.

Another embodiment comprises cyclosporin A, Arlacel 80®, and Clary sage oil.

Another embodiment comprises cyclosporin A, Arlacel C®, and Clary sage oil.

Another embodiment comprises cyclosporin A, Armul 21®, and Clary sage oil.

Another embodiment comprises cyclosporin A, Atlox 80®, and Clary sage oil.

Another embodiment comprises cyclosporin A, Atlox 847®, and Clary sage oil.

Another embodiment comprises cyclosporin A, Atlox 1045A®, and Clary sage oil.

Another embodiment comprises cyclosporin A, Canarcel 20®, and Clary sage oil.

Another embodiment comprises cyclosporin A, Canarcel 60®, and Clary sage oil.

Another embodiment comprises cyclosporin A, Canarcel 80®, and Clary sage oil.

Another embodiment comprises cyclosporin A, Canarcel TW 20®, and Clary sage oil.

Another embodiment comprises cyclosporin A, Canarcel TW 60®, and Clary sage oil.

Another embodiment comprises cyclosporin A, Canarcel TW 80®, and Clary sage oil.

Another embodiment comprises cyclosporin A, Coladet BSB-P®, and Clary sage oil.

Another embodiment comprises cyclosporin A, Customulse O-20®, and Clary sage oil.

Another embodiment comprises cyclosporin A, Dehymuls E®, and Clary sage oil.

Another embodiment comprises cyclosporin A, DeSotan SMO®, and Clary sage oil.

Another embodiment comprises cyclosporin A, DeSotan SMO-20®, and Clary sage oil.

Another embodiment comprises cyclosporin A, DeSotan SMT®, and Clary sage oil.

Another embodiment comprises cyclosporin A, DeSotan SMT-20®, and Clary sage oil.

Another embodiment comprises cyclosporin A, Durfax 60®, and Clary sage oil.

Another embodiment comprises cyclosporin A, Durlax 65®, and Clary sage oil.

Another embodiment comprises cyclosporin A, Durlax 80®, and Clary sage oil.

Another embodiment comprises cyclosporin A, Durtan 60®, and Clary sage oil.

Another embodiment comprises cyclosporin A, Durtan 65®, and Clary sage oil.

Another embodiment comprises cyclosporin A, Liposorb L®, and Clary sage oil.

Another embodiment comprises cyclosporin A, Liposorb L-10®, and Clary sage oil.

Another embodiment comprises cyclosporin A, Liposorb L-20®, and Clary sage oil.

Another embodiment comprises cyclosporin A, Liposorb O®, and Clary sage oil.

Another embodiment comprises cyclosporin A, Liposorb O-20®, and Clary sage oil.

Another embodiment comprises cyclosporin A, Liposorb P®, and Clary sage oil.

Another embodiment comprises cyclosporin A, Liposorb P-20®, and Clary sage oil.

Another embodiment comprises cyclosporin A, Liposorb S®, and Clary sage oil.

Another embodiment comprises cyclosporin A, Liposorb S-20®, and Clary sage oil.

Another embodiment comprises cyclosporin A, Liposorb SQO®, and Clary sage oil.

Another embodiment comprises cyclosporin A, Liposorb TO®, and Clary sage oil.

Another embodiment comprises cyclosporin A, Liposorb TS®, and Clary sage oil.

Another embodiment comprises cyclosporin A, Liposorb TS-20®, and Clary sage oil.

Another embodiment comprises cyclosporin A, Lumisorb PS®, and Clary sage oil.

Another embodiment comprises cyclosporin A, Lumisorb SMO (T)®, and Clary sage oil.

Another embodiment comprises cyclosporin A, Lumisorb SMS K®, and Clary sage oil.

Another embodiment comprises cyclosporin A, Lumisorb SSO®, and Clary sage oil.

Another embodiment comprises cyclosporin A, Lumisorb STS K®, and Clary sage oil.

Another embodiment comprises cyclosporin A, Lumisorb STT®, and Clary sage oil.

Another embodiment comprises cyclosporin A, Magrabar SMO®, and Clary sage oil.

Another embodiment comprises cyclosporin A, Magrabar SMO-VEG®, and Clary sage oil.

Another embodiment comprises cyclosporin A, Magrabar SMT®, and Clary sage oil.

Another embodiment comprises cyclosporin A, Magrabar STO®, and Clary sage oil.

Another embodiment comprises cyclosporin A, Miracare BC-27®, and Clary sage oil.

Another embodiment comprises cyclosporin A, Ritabate 20®, and Clary sage oil.

Another embodiment comprises cyclosporin A, Ritabate 40®, and Clary sage oil.

Another embodiment comprises cyclosporin A, Ritabate 60®, and Clary sage oil.

Another embodiment comprises cyclosporin A, Ritabate 80®, and Clary sage oil.

Another embodiment comprises cyclosporin A, T-Maz®, and Clary sage oil.

Another embodiment comprises cyclosporin A, Tego SML®, and Clary sage oil.

Another embodiment comprises cyclosporin A, Tego SML 20®, and Clary sage oil.

Another embodiment comprises cyclosporin A, Tego SMO 80 V®, and Clary sage oil.

Another embodiment comprises cyclosporin A, Tego SMO V®, and Clary sage oil.

Another embodiment comprises cyclosporin A, Tego SMS®, and Clary sage oil.

Another embodiment comprises cyclosporin A, Tego STO V®, and Clary sage oil.

Another embodiment comprises cyclosporin A, Tween 21®, and Clary sage oil.

Another embodiment comprises cyclosporin A, Tween 40®, and Clary sage oil.

Another embodiment comprises cyclosporin A, Tween 60®, and Clary sage oil.

Another embodiment comprises cyclosporin A, Tween 60 K®, and Clary sage oil.

Another embodiment comprises cyclosporin A, Tween 61®, and Clary sage oil.

Another embodiment comprises cyclosporin A, Tween 65®, and Clary sage oil.

Another embodiment comprises cyclosporin A, Tween 80®, and Clary sage oil.

Another embodiment comprises cyclosporin A, Tween 80 K®, and Clary sage oil.

Another embodiment comprises cyclosporin A, Tween 81®, and Clary sage oil.

Another embodiment comprises cyclosporin A, Tween 85®, and Clary sage oil.

Another embodiment comprises cyclosporin A, a Sucrose or Glucose Ester, or Derivative thereof, and Clary sage oil.

Another embodiment comprises cyclosporin A, DeSulf GOS-P-60WCG®, and Clary sage oil.

Another embodiment comprises cyclosporin A, Glucam E-20 Distearate®, and Clary sage oil.

Another embodiment comprises cyclosporin A, Glucamate DOE-120®, and Clary sage oil.

Another embodiment comprises cyclosporin A, Glucamate SSE-20®, and Clary sage oil.

Another embodiment comprises cyclosporin A, Glucate DO®, and Clary sage oil.

Another embodiment comprises cyclosporin A, Glucate SS®, and Clary sage oil.

Another embodiment comprises cyclosporin A, Glucopon 425 UP®, and Clary sage oil.

Another embodiment comprises cyclosporin A, Isolan IS®, and Clary sage oil.

Another embodiment comprises cyclosporin A, Mazon 40®, and Clary sage oil.

Another embodiment comprises cyclosporin A, Montanov 82®, and Clary sage oil.

Another embodiment comprises cyclosporin A, Montanov 202®, and Clary sage oil.

Another embodiment comprises cyclosporin A, Montanov S®, and Clary sage oil.

Another embodiment comprises cyclosporin A, Rheozan®, and Clary sage oil.

Another embodiment comprises cyclosporin A, Simulsol AS 48®, and Clary sage oil.

Another embodiment comprises cyclosporin A, Simulsol SL 4®, and Clary sage oil.

Another embodiment comprises cyclosporin A, Simulsol SL 10®, and Clary sage oil.

Another embodiment comprises cyclosporin A, Simulsol SL 11W®, and Clary sage oil.

Another embodiment comprises cyclosporin A, Simulsol SL 55®, and Clary sage oil.

Another embodiment comprises cyclosporin A, Suga Nate 100 and 160®, and Clary sage oil.

Another embodiment comprises cyclosporin A, Tego Care 450®, and Clary sage oil.

Another embodiment comprises cyclosporin A, Tego Care CG 90®, and Clary sage oil.

Another embodiment comprises cyclosporin A, Tego Care PS®, and Clary sage oil.

Another embodiment comprises cyclosporin A, Tegosoft PSE 141 G®, and Clary sage oil.

Another embodiment comprises cyclosporin A, Tegotens G 826®, and Clary sage oil.

Another embodiment comprises cyclosporin A, Triton BG-10 (70%)®, and Clary sage oil.

Another embodiment comprises cyclosporin A, Triton CG-110 (60%)®, and Clary sage oil.

Another embodiment comprises cyclosporin A, Wickenol 545®, and Clary sage oil.

Another embodiment comprises cyclosporin A, an alcohol, and Nutmeg oil.

Another embodiment comprises cyclosporin A, Diglycerol®, and Nutmeg oil.

Another embodiment comprises cyclosporin A, Hetoxide GT-80®, and Nutmeg oil.

Another embodiment comprises cyclosporin A, Lexemul BEO®, and Nutmeg oil.

Another embodiment comprises cyclosporin A, Polyglycerol-3®, and Nutmeg oil.

Another embodiment comprises cyclosporin A, Redicote E Series®, and Nutmeg oil.

Another embodiment comprises cyclosporin A, Simulsol OX 1005L®, and Nutmeg oil.

Another embodiment comprises cyclosporin A, Stanfax 567®, and Nutmeg oil.

Another embodiment comprises cyclosporin A, TA-1618®, and Nutmeg oil.

Another embodiment comprises cyclosporin A, Witconol H-31A®, and Nutmeg oil.

Another embodiment comprises cyclosporin A, an amine oxide, and Nutmeg oil.

Another embodiment comprises cyclosporin A, AO-405®, and Nutmeg oil.

Another embodiment comprises cyclosporin A, AO-455®, and Nutmeg oil.

Another embodiment comprises cyclosporin A, AO 728 Special®, and Nutmeg oil.

Another embodiment comprises cyclosporin A, Barlox 12®, and Nutmeg oil.

Another embodiment comprises cyclosporin A, Barlox 14®, and Nutmeg oil.

Another embodiment comprises cyclosporin A, Burcoxide Lo®, and Nutmeg oil.

Another embodiment comprises cyclosporin A, Caloxamine LO®, and Nutmeg oil.

Another embodiment comprises cyclosporin A, Chemoxide CAW®, and Nutmeg oil.

Another embodiment comprises cyclosporin A, Chemoxide LM-30®, and Nutmeg oil.

Another embodiment comprises cyclosporin A, Chemoxide LO®, and Nutmeg oil.

Another embodiment comprises cyclosporin A, Chemoxide MO®, and Nutmeg oil.

Another embodiment comprises cyclosporin A, Colalux CAO-35®, and Nutmeg oil.

Another embodiment comprises cyclosporin A, Colalux LO®, and Nutmeg oil.

Another embodiment comprises cyclosporin A, DeMox CAPO®, and Nutmeg oil.

Another embodiment comprises cyclosporin A, DeMox CSG-30®, and Nutmeg oil.

Another embodiment comprises cyclosporin A, DeMox LAO®, and Nutmeg oil.

Another embodiment comprises cyclosporin A, Emcol L®, and Nutmeg oil.

Another embodiment comprises cyclosporin A, Empigen OB®, and Nutmeg oil.

Another embodiment comprises cyclosporin A, Empigen OS/A®, and Nutmeg oil.

Another embodiment comprises cyclosporin A, Foamox CDO®, and Nutmeg oil.

Another embodiment comprises cyclosporin A, Foamox DMM®, and Nutmeg oil.

Another embodiment comprises cyclosporin A, Foamox DMS®, and Nutmeg oil.

Another embodiment comprises cyclosporin A, Genaminox KC®, and Nutmeg oil.

Another embodiment comprises cyclosporin A, Genaminox LA®, and Nutmeg oil.

Another embodiment comprises cyclosporin A, Hartofoam SAO®, and Nutmeg oil.

Another embodiment comprises cyclosporin A, Hartox DMCD®, and Nutmeg oil.

Another embodiment comprises cyclosporin A, Lipowax DAT®, and Nutmeg oil.

Another embodiment comprises cyclosporin A, Lipowax PB Pastilles®, and Nutmeg oil.

Another embodiment comprises cyclosporin A, Mackamine C8®, and Nutmeg oil.

Another embodiment comprises cyclosporin A, Mackamine C10®, and Nutmeg oil.

Another embodiment comprises cyclosporin A, Mackamine C14®, and Nutmeg oil.

Another embodiment comprises cyclosporin A, Mackamine CAO®, and Nutmeg oil.

Another embodiment comprises cyclosporin A, Mackamine CO®, and Nutmeg oil.

Another embodiment comprises cyclosporin A, Mackamine LO®, and Nutmeg oil.

Another embodiment comprises cyclosporin A, Mackamine O2®, and Nutmeg oil.

Another embodiment comprises cyclosporin A, Mackamine SAO®, and Nutmeg oil.

Another embodiment comprises cyclosporin A, Mackamine SO®, and Nutmeg oil.

Another embodiment comprises cyclosporin A, Mazox KCAO®, and Nutmeg oil.

Another embodiment comprises cyclosporin A, Monalac MO®, and Nutmeg oil.

Another embodiment comprises cyclosporin A, Norfox LDA®, and Nutmeg oil.

Another embodiment comprises cyclosporin A, Rhodamox LO®, and Nutmeg oil.

Another embodiment comprises cyclosporin A, Schercamox C-AA®, and Nutmeg oil.

Another embodiment comprises cyclosporin A, Schercamox DMA®, and Nutmeg oil.

Another embodiment comprises cyclosporin A, Schercamox DML®, and Nutmeg oil.

Another embodiment comprises cyclosporin A, Schercamox DMM®, and Nutmeg oil.

Another embodiment comprises cyclosporin A, Schercamox DMS®, and Nutmeg oil.

Another embodiment comprises cyclosporin A, Tegotens DO®, and Nutmeg oil.

Another embodiment comprises cyclosporin A, Tomah AO-14-2®, and Nutmeg oil.

Another embodiment comprises cyclosporin A, Triaminox CDO®, and Nutmeg oil.

Another embodiment comprises cyclosporin A, a block polymer, and Nutmeg oil.

Another embodiment comprises cyclosporin A, AL 2070®, and Nutmeg oil.

Another embodiment comprises cyclosporin A, Antarox 17-R-2®, and Nutmeg oil.

Another embodiment comprises cyclosporin A, Antarox 25-R-2®, and Nutmeg oil.

Another embodiment comprises cyclosporin A, Antarox 31-R-1®, and Nutmeg oil.

Another embodiment comprises cyclosporin A, Antarox P-84®, and Nutmeg oil.

Another embodiment comprises cyclosporin A, Antarox P-104/H®, and Nutmeg oil.

Another embodiment comprises cyclosporin A, Arnox BP-Series®, and Nutmeg oil.

Another embodiment comprises cyclosporin A, Chemonic 435®, and Nutmeg oil.

Another embodiment comprises cyclosporin A, Chemonic D-25®, and Nutmeg oil.

Another embodiment comprises cyclosporin A, Chemonic PL Series®, and Nutmeg oil.

Another embodiment comprises cyclosporin A, Ethox L-121®, and Nutmeg oil.

Another embodiment comprises cyclosporin A, Ethox L-122®, and Nutmeg oil.

Another embodiment comprises cyclosporin A, Genapol PF-10®, and Nutmeg oil.

Another embodiment comprises cyclosporin A, Genapol PF-20®, and Nutmeg oil.

Another embodiment comprises cyclosporin A, Genapol PF-40A®, and Nutmeg oil.

Another embodiment comprises cyclosporin A, Norfox 2-LF®, and Nutmeg oil.

Another embodiment comprises cyclosporin A, Simulsol NW 342®, and Nutmeg oil.

Another embodiment comprises cyclosporin A, T-Det BP-1®, and Nutmeg oil.

Another embodiment comprises cyclosporin A, T-Det XD®, and Nutmeg oil.

Another embodiment comprises cyclosporin A, T-Det XH®, and Nutmeg oil.

Another embodiment comprises cyclosporin A, Triton CF-32®, and Nutmeg oil.

Another embodiment comprises cyclosporin A, Witconol 171®, and Nutmeg oil.

Another embodiment comprises cyclosporin A, Witconol 324®, and Nutmeg oil.

Another embodiment comprises cyclosporin A, Witconol 324D®, and Nutmeg oil.

Another embodiment comprises cyclosporin A, Witconol PD-2000®, and Nutmeg oil.

Another embodiment comprises cyclosporin A, a Carboxylated Alcohol or Alkylphenol Ethoxylate, and Nutmeg oil.

Another embodiment comprises cyclosporin A, Emcol CN-6®, and Nutmeg oil.

Another embodiment comprises cyclosporin A, Ethcarb®, and Nutmeg oil.

Another embodiment comprises cyclosporin A, Gemtex WNT-Conc®, and Nutmeg oil.

Another embodiment comprises cyclosporin A, Incrodet TD7-C®, and Nutmeg oil.

Another embodiment comprises cyclosporin A, Marlinat CM 105/80®, and Nutmeg oil.

Another embodiment comprises cyclosporin A, Marlowet 1072®, and Nutmeg oil.

Another embodiment comprises cyclosporin A, Marlowet 4530®, and Nutmeg oil.

Another embodiment comprises cyclosporin A, Marlowet 4530 LF®, and Nutmeg oil.

Another embodiment comprises cyclosporin A, Marlowet 4534®, and Nutmeg oil.

Another embodiment comprises cyclosporin A, Marlowet 4538®, and Nutmeg oil.

Another embodiment comprises cyclosporin A, Marlowet 4539®, and Nutmeg oil.

Another embodiment comprises cyclosporin A, Marlowet 4539 LF®, and Nutmeg oil.

Another embodiment comprises cyclosporin A, Marlowet 4541®, and Nutmeg oil.

Another embodiment comprises cyclosporin A, Miranate LEC-80®, and Nutmeg oil.

Another embodiment comprises cyclosporin A, Sandopan B®, and Nutmeg oil.

Another embodiment comprises cyclosporin A, Sandopan B Modified®, and Nutmeg oil.

Another embodiment comprises cyclosporin A, Sandopan LS-24 Gel®, and Nutmeg oil.

Another embodiment comprises cyclosporin A, Surfine T-A®, and Nutmeg oil.

Another embodiment comprises cyclosporin A, a Carboxylic Acid/Fatty Acid, and Nutmeg oil.

Another embodiment comprises cyclosporin A, Colaterge RAM®, and Nutmeg oil.

Another embodiment comprises cyclosporin A, Colatrope INC®, and Nutmeg oil.

Another embodiment comprises cyclosporin A, Crodacid B®, and Nutmeg oil.

Another embodiment comprises cyclosporin A, DeTrope CA-100®, and Nutmeg oil.

Another embodiment comprises cyclosporin A, Latol MTO®, and Nutmeg oil.

Another embodiment comprises cyclosporin A, Lumulse CC-33 K®, and Nutmeg oil.

Another embodiment comprises cyclosporin A, Mulls 2218®, and Nutmeg oil.

Another embodiment comprises cyclosporin A, OL-600®, and Nutmeg oil.

Another embodiment comprises cyclosporin A, OL-800®, and Nutmeg oil.

Another embodiment comprises cyclosporin A, R-910®, and Nutmeg oil.

Another embodiment comprises cyclosporin A, S-210®, and Nutmeg oil.

Another embodiment comprises cyclosporin A, Sandopan DTC Acid®, and Nutmeg oil.

Another embodiment comprises cyclosporin A, Sandopan LS 24 N®, and Nutmeg oil.

Another embodiment comprises cyclosporin A, Sandopan MA-18®, and Nutmeg oil.

Another embodiment comprises cyclosporin A, an Ethoxylated Alcohol, and Nutmeg oil.

Another embodiment comprises cyclosporin A, Adsee 799®, and Nutmeg oil.

Another embodiment comprises cyclosporin A, Adsee 799®, and Nutmeg oil.

Another embodiment comprises cyclosporin A, Alfonic 610-3.5®, and Nutmeg oil.

Another embodiment comprises cyclosporin A, Alfonic 810-2®, and Nutmeg oil.

Another embodiment comprises cyclosporin A, Alfonic 810-6®, and Nutmeg oil.

Another embodiment comprises cyclosporin A, Alfonic 1012-3®, and Nutmeg oil.

Another embodiment comprises cyclosporin A, Alfonic 1012-5®, and Nutmeg oil.

Another embodiment comprises cyclosporin A, Alfonic 1216CO-1.5®, and Nutmeg oil.

Another embodiment comprises cyclosporin A, Alfonic 1216CO-7®, and Nutmeg oil.

Another embodiment comprises cyclosporin A, Alfonic 1412-3®, and Nutmeg oil.

Another embodiment comprises cyclosporin A, Alfonic 1412-7®, and Nutmeg oil.

Another embodiment comprises cyclosporin A, Arlasolve 200®, and Nutmeg oil.

Another embodiment comprises cyclosporin A, Arlasolve 200 Liquid®, and Nutmeg oil.

Another embodiment comprises cyclosporin A, Armix 180-C®, and Nutmeg oil.

Another embodiment comprises cyclosporin A, Armix 183®, and Nutmeg oil.

Another embodiment comprises cyclosporin A, Armul 2404®, and Nutmeg oil.

Another embodiment comprises cyclosporin A, Armul 2404®, and Nutmeg oil.

Another embodiment comprises cyclosporin A, Atlas EMJ-C®, and Nutmeg oil.

Another embodiment comprises cyclosporin A, Atlas G-2109®, and Nutmeg oil.

Another embodiment comprises cyclosporin A, Atlas G-3886®, and Nutmeg oil.

Another embodiment comprises cyclosporin A, Atlas G-3890®, and Nutmeg oil.

Another embodiment comprises cyclosporin A, Bio Soft E-200®, and Nutmeg oil.

Another embodiment comprises cyclosporin A, Bio Soft E-300®, and Nutmeg oil.

Another embodiment comprises cyclosporin A, Bio Soft E-400®, and Nutmeg oil.

Another embodiment comprises cyclosporin A, Bio Soft EN 600®, and Nutmeg oil.

Another embodiment comprises cyclosporin A, Bio Soft TD-400®, and Nutmeg oil.

Another embodiment comprises cyclosporin A, Bio Soft TD-630®, and Nutmeg oil.

Another embodiment comprises cyclosporin A, Brij 30®, and Nutmeg oil.

Another embodiment comprises cyclosporin A, Brij 52®, and Nutmeg oil.

Another embodiment comprises cyclosporin A, Brij 56®, and Nutmeg oil.

Another embodiment comprises cyclosporin A, Brij 58®, and Nutmeg oil.

Another embodiment comprises cyclosporin A, Brij 72®, and Nutmeg oil.

Another embodiment comprises cyclosporin A, Brij 76®, and Nutmeg oil.

Another embodiment comprises cyclosporin A, Brij 78®, and Nutmeg oil.

Another embodiment comprises cyclosporin A, Brij 93®, and Nutmeg oil.

Another embodiment comprises cyclosporin A, Brij 97®, and Nutmeg oil.

Another embodiment comprises cyclosporin A, Brij 98®, and Nutmeg oil.

Another embodiment comprises cyclosporin A, Brij 700®, and Nutmeg oil.

Another embodiment comprises cyclosporin A, Brij 700 S®, and Nutmeg oil.

Another embodiment comprises cyclosporin A, Brij 721®, and Nutmeg oil.

Another embodiment comprises cyclosporin A, Brij 721 S®, and Nutmeg oil.

Another embodiment comprises cyclosporin A, Burcoterge CDG®, and Nutmeg oil.

Another embodiment comprises cyclosporin A, Canasol AT 600®, and Nutmeg oil.

Another embodiment comprises cyclosporin A, Canasol AT 800®, and Nutmeg oil.

Another embodiment comprises cyclosporin A, Canasol AT 1200®, and Nutmeg oil.

Another embodiment comprises cyclosporin A, Canasol BJ 35®, and Nutmeg oil.

Another embodiment comprises cyclosporin A, Canasol BJ 36®, and Nutmeg oil.

Another embodiment comprises cyclosporin A, Canasol BJ 52®, and Nutmeg oil.

Another embodiment comprises cyclosporin A, Canasol BJ 58®, and Nutmeg oil.

Another embodiment comprises cyclosporin A, Canasol BJ 72®, and Nutmeg oil.

Another embodiment comprises cyclosporin A, Canasol BJ 78®, and Nutmeg oil.

Another embodiment comprises cyclosporin A, Canasol BJ 98®, and Nutmeg oil.

Another embodiment comprises cyclosporin A, Canasol BJ 307®, and Nutmeg oil.

Another embodiment comprises cyclosporin A, Cerlak 1400®, and Nutmeg oil.

Another embodiment comprises cyclosporin A, Cetomacrogol 1000 BP®, and Nutmeg oil.

Another embodiment comprises cyclosporin A, Chemonic C-2®, and Nutmeg oil.

Another embodiment comprises cyclosporin A, Chemonic C-10®, and Nutmeg oil.

Another embodiment comprises cyclosporin A, Chemonic C-20®, and Nutmeg oil.

Another embodiment comprises cyclosporin A, Chemonic CT-12®, and Nutmeg oil.

Another embodiment comprises cyclosporin A, Chemonic CT-20®, and Nutmeg oil.

Another embodiment comprises cyclosporin A, Chemonic CT-30®, and Nutmeg oil.

Another embodiment comprises cyclosporin A, Chemonic CT-55®, and Nutmeg oil.

Another embodiment comprises cyclosporin A, Chemonic G-7®, and Nutmeg oil.

Another embodiment comprises cyclosporin A, Chemonic G-26®, and Nutmeg oil.

Another embodiment comprises cyclosporin A, Chemonic L-4®, and Nutmeg oil.

Another embodiment comprises cyclosporin A, Chemonic L-7®, and Nutmeg oil.

Another embodiment comprises cyclosporin A, Chemonic L-12®, and Nutmeg oil.

Another embodiment comprises cyclosporin A, Chemonic L-23®, and Nutmeg oil.

Another embodiment comprises cyclosporin A, Chemonic O-2®, and Nutmeg oil.

Another embodiment comprises cyclosporin A, Chemonic O-5®, and Nutmeg oil.

Another embodiment comprises cyclosporin A, Chemonic O-10®, and Nutmeg oil.

Another embodiment comprises cyclosporin A, Chemonic O-20®, and Nutmeg oil.

Another embodiment comprises cyclosporin A, Chemonic S-2®, and Nutmeg oil.

Another embodiment comprises cyclosporin A, Chemonic S-10®, and Nutmeg oil.

Another embodiment comprises cyclosporin A, Chemonic S-20®, and Nutmeg oil.

Another embodiment comprises cyclosporin A, Colamulse FE®, and Nutmeg oil.

Another embodiment comprises cyclosporin A, Cremophor A 20®, and Nutmeg oil.

Another embodiment comprises cyclosporin A, Cremophor SA 2®, and Nutmeg oil.

Another embodiment comprises cyclosporin A, Dehydol 100®, and Nutmeg oil.

Another embodiment comprises cyclosporin A, Dehydol O-4®, and Nutmeg oil.

Another embodiment comprises cyclosporin A, Delonic C-18®, and Nutmeg oil.

Another embodiment comprises cyclosporin A, DeSonic 6T®, and Nutmeg oil.

Another embodiment comprises cyclosporin A, DeSonic 9D®, and Nutmeg oil.

Another embodiment comprises cyclosporin A, DeSonic 9T®, and Nutmeg oil.

Another embodiment comprises cyclosporin A, DeSonic 12D®, and Nutmeg oil.

Another embodiment comprises cyclosporin A, DeSonic 12T®, and Nutmeg oil.

Another embodiment comprises cyclosporin A, DeSonic 15T®, and Nutmeg oil.

Another embodiment comprises cyclosporin A, DeSonic TDA-9®, and Nutmeg oil.

Another embodiment comprises cyclosporin A, DeThox GLG-7®, and Nutmeg oil.

Another embodiment comprises cyclosporin A, DeThox GLG-26®, and Nutmeg oil.

Another embodiment comprises cyclosporin A, DeThox LA-4®, and Nutmeg oil.

Another embodiment comprises cyclosporin A, DeThox LA-23®, and Nutmeg oil.

Another embodiment comprises cyclosporin A, DeThox SA-80®, and Nutmeg oil.

Another embodiment comprises cyclosporin A, Disponil O5®, and Nutmeg oil.

Another embodiment comprises cyclosporin A, Eccoterge EO-41 B®, and Nutmeg oil.

Another embodiment comprises cyclosporin A, Empilan KA2.5/90®, and Nutmeg oil.

Another embodiment comprises cyclosporin A, Empilan KA5/90®, and Nutmeg oil.

Another embodiment comprises cyclosporin A, Empilan KM-20®, and Nutmeg oil.

Another embodiment comprises cyclosporin A, Empilan KM-50®, and Nutmeg oil.

Another embodiment comprises cyclosporin A, Empilan L-23®, and Nutmeg oil.

Another embodiment comprises cyclosporin A, Ethylan 25-3®, and Nutmeg oil.

Another embodiment comprises cyclosporin A, Ethylan 1204®, and Nutmeg oil.

Another embodiment comprises cyclosporin A, Ethylan DA-4®, and Nutmeg oil.

Another embodiment comprises cyclosporin A, Ethylan LA-230®, and Nutmeg oil.

Another embodiment comprises cyclosporin A, Ethylan SN®, and Nutmeg oil.

Another embodiment comprises cyclosporin A, Ethylan TD-60®, and Nutmeg oil.

Another embodiment comprises cyclosporin A, Ethylan TD-100®, and Nutmeg oil.

Another embodiment comprises cyclosporin A, Ethylan TD-1407®, and Nutmeg oil.

Another embodiment comprises cyclosporin A, Eumulgin B1®, and Nutmeg oil.

Another embodiment comprises cyclosporin A, Eumulgin B2®, and Nutmeg oil.

Another embodiment comprises cyclosporin A, Eumulgin B3®, and Nutmeg oil.

Another embodiment comprises cyclosporin A, Eumulgin O-10®, and Nutmeg oil.

Another embodiment comprises cyclosporin A, Flo Mo 80/20®, and Nutmeg oil.

Another embodiment comprises cyclosporin A, Flo Mo Low Foam®, and Nutmeg oil.

Another embodiment comprises cyclosporin A, Forlan C-24®, and Nutmeg oil.

Another embodiment comprises cyclosporin A, Genapol 1454®, and Nutmeg oil.

Another embodiment comprises cyclosporin A, Genapol BA-020®, and Nutmeg oil.

Another embodiment comprises cyclosporin A, Genapol BA-040®, and Nutmeg oil.

Another embodiment comprises cyclosporin A, Genapol C-100®, and Nutmeg oil.

Another embodiment comprises cyclosporin A, Genapol DA 060®, and Nutmeg oil.

Another embodiment comprises cyclosporin A, Genapol HS 020®, and Nutmeg oil.

Another embodiment comprises cyclosporin A, Genapol HS 200®, and Nutmeg oil.

Another embodiment comprises cyclosporin A, Genapol ID-040®, and Nutmeg oil.

Another embodiment comprises cyclosporin A, Genapol ID-060®, and Nutmeg oil.

Another embodiment comprises cyclosporin A, Genapol ID-090®, and Nutmeg oil.

Another embodiment comprises cyclosporin A, Genapol LA 010®, and Nutmeg oil.

Another embodiment comprises cyclosporin A, Genapol LA 020®, and Nutmeg oil.

Another embodiment comprises cyclosporin A, Genapol LA 030®, and Nutmeg oil.

Another embodiment comprises cyclosporin A, Genapol LA 040®, and Nutmeg oil.

Another embodiment comprises cyclosporin A, Genapol LA 050®, and Nutmeg oil.

Another embodiment comprises cyclosporin A, Genapol LA 060®, and Nutmeg oil.

Another embodiment comprises cyclosporin A, Genapol LA 070®, and Nutmeg oil.

Another embodiment comprises cyclosporin A, Genapol LA 070S®, and Nutmeg oil.

Another embodiment comprises cyclosporin A, Genapol LA 230®, and Nutmeg oil.

Another embodiment comprises cyclosporin A, Genapol O 020®, and Nutmeg oil.

Another embodiment comprises cyclosporin A, Genapol O 050®, and Nutmeg oil.

Another embodiment comprises cyclosporin A, Genapol O 100®, and Nutmeg oil.

Another embodiment comprises cyclosporin A, Genapol O 200®, and Nutmeg oil.

Another embodiment comprises cyclosporin A, Genapol SA 030®, and Nutmeg oil.

Another embodiment comprises cyclosporin A, Genapol SA 120®, and Nutmeg oil.

Another embodiment comprises cyclosporin A, Genapol T-020®, and Nutmeg oil.

Another embodiment comprises cyclosporin A, Genapol UD-030®, and Nutmeg oil.

Another embodiment comprises cyclosporin A, Genapol UD-050®, and Nutmeg oil.

Another embodiment comprises cyclosporin A, Genapol UD-070®, and Nutmeg oil.

Another embodiment comprises cyclosporin A, Genapol UD-079®, and Nutmeg oil.

Another embodiment comprises cyclosporin A, Genapol UD-080®, and Nutmeg oil.

Another embodiment comprises cyclosporin A, Genapol UD-110®, and Nutmeg oil.

Another embodiment comprises cyclosporin A, Genapol X 030®, and Nutmeg oil.

Another embodiment comprises cyclosporin A, Genapol X 050®, and Nutmeg oil.

Another embodiment comprises cyclosporin A, Genapol X 060®, and Nutmeg oil.

Another embodiment comprises cyclosporin A, Genapol X 070®, and Nutmeg oil.

Another embodiment comprises cyclosporin A, Genapol X 080®, and Nutmeg oil.

Another embodiment comprises cyclosporin A, Genapol X 100®, and Nutmeg oil.

Another embodiment comprises cyclosporin A, Genapol X159®, and Nutmeg oil.

Another embodiment comprises cyclosporin A, Generol 122 E5®, and Nutmeg oil.

Another embodiment comprises cyclosporin A, Generol 122 E25®, and Nutmeg oil.

Another embodiment comprises cyclosporin A, Hostacerin T-3®, and Nutmeg oil.

Another embodiment comprises cyclosporin A, Iconol LF 110®, and Nutmeg oil.

Another embodiment comprises cyclosporin A, Incropol CS-20®, and Nutmeg oil.

Another embodiment comprises cyclosporin A, Lexemul CS-20®, and Nutmeg oil.

Another embodiment comprises cyclosporin A, Liponic EG-1®, and Nutmeg oil.

Another embodiment comprises cyclosporin A, Lipowax D®, and Nutmeg oil.

Another embodiment comprises cyclosporin A, Lipowax G®, and Nutmeg oil.

Another embodiment comprises cyclosporin A, Lipowax NI®, and Nutmeg oil.

Another embodiment comprises cyclosporin A, Lipowax P®, and Nutmeg oil.

Another embodiment comprises cyclosporin A, Lipowax P-31®, and Nutmeg oil.

Another embodiment comprises cyclosporin A, Lipowax PR®, and Nutmeg oil.

Another embodiment comprises cyclosporin A, Lumulse CS-20®, and Nutmeg oil.

Another embodiment comprises cyclosporin A, Macol CSA-20®, and Nutmeg oil.

Another embodiment comprises cyclosporin A, Marlox B 24/50®, and Nutmeg oil.

Another embodiment comprises cyclosporin A, Mazawet 77®, and Nutmeg oil.

Another embodiment comprises cyclosporin A, Norfox 1713®, and Nutmeg oil.

Another embodiment comprises cyclosporin A, Norfox 2579®, and Nutmeg oil.

Another embodiment comprises cyclosporin A, Norfox Lo Foam®, and Nutmeg oil.

Another embodiment comprises cyclosporin A, Promulgen D®, and Nutmeg oil.

Another embodiment comprises cyclosporin A, Promulgen G®, and Nutmeg oil.

Another embodiment comprises cyclosporin A, Renex 30®, and Nutmeg oil.

Another embodiment comprises cyclosporin A, Renex 36®, and Nutmeg oil.

Another embodiment comprises cyclosporin A, Rhodasud A 24®, and Nutmeg oil.

Another embodiment comprises cyclosporin A, Rhodasurf AAE-10®, and Nutmeg oil.

Another embodiment comprises cyclosporin A, Rhodasurf BEH-25®, and Nutmeg oil.

Another embodiment comprises cyclosporin A, Rhodasurf BEH-40®, and Nutmeg oil.

Another embodiment comprises cyclosporin A, Rhodasurf DA 530®, and Nutmeg oil.

Another embodiment comprises cyclosporin A, Rhodasurf DA 630®, and Nutmeg oil.

Another embodiment comprises cyclosporin A, Rhodasurf DA 639®, and Nutmeg oil.

Another embodiment comprises cyclosporin A, Rhodasurf LAN-23®, and Nutmeg oil.

Another embodiment comprises cyclosporin A, Rhodasurf ON-870®, and Nutmeg oil.

Another embodiment comprises cyclosporin A, Rhodasurf ON-877®, and Nutmeg oil.

Another embodiment comprises cyclosporin A, Rhodasurf TB-970 FLK®, and Nutmeg oil.

Another embodiment comprises cyclosporin A, Ritacet-20®, and Nutmeg oil.

Another embodiment comprises cyclosporin A, Ritachol 1000®, and Nutmeg oil.

Another embodiment comprises cyclosporin A, Ritachol 2000®, and Nutmeg oil.

Another embodiment comprises cyclosporin A, Ritachol 5000®, and Nutmeg oil.

Another embodiment comprises cyclosporin A, Ritox 35®, and Nutmeg oil.

Another embodiment comprises cyclosporin A, Surfonic DA-4®, and Nutmeg oil.

Another embodiment comprises cyclosporin A, Surfonic DA-6®, and Nutmeg oil.

Another embodiment comprises cyclosporin A, Surfonic L46-7®, and Nutmeg oil.

Another embodiment comprises cyclosporin A, Surfonic POA®, and Nutmeg oil.

Another embodiment comprises cyclosporin A, Synthrapol KB®, and Nutmeg oil.

Another embodiment comprises cyclosporin A, Teginacid®, and Nutmeg oil.

Another embodiment comprises cyclosporin A, Teginacid C®, and Nutmeg oil.

Another embodiment comprises cyclosporin A, Tegotens EC 11®, and Nutmeg oil.

Another embodiment comprises cyclosporin A, Tinegal NA®, and Nutmeg oil.

Another embodiment comprises cyclosporin A, Tomadol 400®, and Nutmeg oil.

Another embodiment comprises cyclosporin A, Tomadol 600®, and Nutmeg oil.

Another embodiment comprises cyclosporin A, Tomadol 900®, and Nutmeg oil.

Another embodiment comprises cyclosporin A, Uniperol O®, and Nutmeg oil.

Another embodiment comprises cyclosporin A, Witconol SN Series®, and Nutmeg oil.

Another embodiment comprises cyclosporin A, an Ethoxylated Alkylphenol, and Nutmeg oil.

Another embodiment comprises cyclosporin A, Antarox LF-222®, and Nutmeg oil.

Another embodiment comprises cyclosporin A, Atlox 775®, and Nutmeg oil.

Another embodiment comprises cyclosporin A, Caloxylate N-9®, and Nutmeg oil.

Another embodiment comprises cyclosporin A, Canasol NF-1000®, and Nutmeg oil.

Another embodiment comprises cyclosporin A, Canasol NF-3000®, and Nutmeg oil.

Another embodiment comprises cyclosporin A, Canasol NF-3070®, and Nutmeg oil.

Another embodiment comprises cyclosporin A, Canasol OF 1670®, and Nutmeg oil.

Another embodiment comprises cyclosporin A, Canasol OF 2570®, and Nutmeg oil.

Another embodiment comprises cyclosporin A, Canasol OF 4070®, and Nutmeg oil.

Another embodiment comprises cyclosporin A, Chemax DNP-8®, and Nutmeg oil.

Another embodiment comprises cyclosporin A, Chemax DNP-18®, and Nutmeg oil.

Another embodiment comprises cyclosporin A, Chemax DNP-150/50®, and Nutmeg oil.

Another embodiment comprises cyclosporin A, DeSonic 1.5N®, and Nutmeg oil.

Another embodiment comprises cyclosporin A, DeSonic 4N®, and Nutmeg oil.

Another embodiment comprises cyclosporin A, DeSonic 5N®, and Nutmeg oil.

Another embodiment comprises cyclosporin A, DeSonic 6D®, and Nutmeg oil.

Another embodiment comprises cyclosporin A, DeSonic 6N®, and Nutmeg oil.

Another embodiment comprises cyclosporin A, DeSonic 7N®, and Nutmeg oil.

Another embodiment comprises cyclosporin A, DeSonic 9N®, and Nutmeg oil.

Another embodiment comprises cyclosporin A, DeSonic 10D®, and Nutmeg oil.

Another embodiment comprises cyclosporin A, DeSonic 11N®, and Nutmeg oil.

Another embodiment comprises cyclosporin A, DeSonic 12N®, and Nutmeg oil.

Another embodiment comprises cyclosporin A, DeSonic 15N®, and Nutmeg oil.

Another embodiment comprises cyclosporin A, DeSonic 20N®, and Nutmeg oil.

Another embodiment comprises cyclosporin A, Eccoscour RC®, and Nutmeg oil.

Another embodiment comprises cyclosporin A, Eccoterge EO-100®, and Nutmeg oil.

Another embodiment comprises cyclosporin A, Emulsifier 632/90%®, and Nutmeg oil.

Another embodiment comprises cyclosporin A, Geronol AG-821®, and Nutmeg oil.

Another embodiment comprises cyclosporin A, Gradonic N-95®, and Nutmeg oil.

Another embodiment comprises cyclosporin A, Hetoxide NP-4®, and Nutmeg oil.

Another embodiment comprises cyclosporin A, Hetoxide NP-30®, and Nutmeg oil.

Another embodiment comprises cyclosporin A, Hostapal N-100®, and Nutmeg oil.

Another embodiment comprises cyclosporin A, Hostapal N-110®, and Nutmeg oil.

Another embodiment comprises cyclosporin A, Igepal CTA-639W®, and Nutmeg oil.

Another embodiment comprises cyclosporin A, Igepal DAP-9®, and Nutmeg oil.

Another embodiment comprises cyclosporin A, Igepal OD-410®, and Nutmeg oil.

Another embodiment comprises cyclosporin A, Igepal SS-837®, and Nutmeg oil.

Another embodiment comprises cyclosporin A, Lipocol NP-9 USP®, and Nutmeg oil.

Another embodiment comprises cyclosporin A, Macol DNP-10®, and Nutmeg oil.

Another embodiment comprises cyclosporin A, Marlophen NP 5®, and Nutmeg oil.

Another embodiment comprises cyclosporin A, Marlophen P 1®, and Nutmeg oil.

Another embodiment comprises cyclosporin A, Sunfonic NB®, and Nutmeg oil.

Another embodiment comprises cyclosporin A, Surfonic OPB-307®, and Nutmeg oil.

Another embodiment comprises cyclosporin A, Surfonic OPB-407®, and Nutmeg oil.

Another embodiment comprises cyclosporin A, Syn Fac 334®, and Nutmeg oil.

Another embodiment comprises cyclosporin A, Syn Fac 8216®, and Nutmeg oil.

Another embodiment comprises cyclosporin A, Triton N-57®, and Nutmeg oil.

Another embodiment comprises cyclosporin A, Trycol 6956®, and Nutmeg oil.

Another embodiment comprises cyclosporin A, Trycol 6961®, and Nutmeg oil.

Another embodiment comprises cyclosporin A, Trycol 6964®, and Nutmeg oil.

Another embodiment comprises cyclosporin A, Trycol 6969®, and Nutmeg oil.

Another embodiment comprises cyclosporin A, Trycol 6974®, and Nutmeg oil.

Another embodiment comprises cyclosporin A, Witbreak DRB-127®, and Nutmeg oil.

Another embodiment comprises cyclosporin A, Witbreak DRB-127®, and Nutmeg oil.

Another embodiment comprises cyclosporin A, Witconol NP Series®, and Nutmeg oil.

Another embodiment comprises cyclosporin A, an Ethoxylated Aryl Phenol, and Nutmeg oil.

Another embodiment comprises cyclosporin A, Soprophor BSU®, and Nutmeg oil.

Another embodiment comprises cyclosporin A, Soprophor CY/8®, and Nutmeg oil.

Another embodiment comprises cyclosporin A, Soprophor S/25®, and Nutmeg oil.

Another embodiment comprises cyclosporin A, Witconol NIO®, and Nutmeg oil.

Another embodiment comprises cyclosporin A, Witconol NIW®, and Nutmeg oil.

Another embodiment comprises cyclosporin A, Witconol S-100®, and Nutmeg oil.

Another embodiment comprises cyclosporin A, an Ethoxylated Fatty Acid, and Nutmeg oil.

Another embodiment comprises cyclosporin A, Aldo PGHMS®, and Nutmeg oil.

Another embodiment comprises cyclosporin A, Alkamuls TO-15/HR®, and Nutmeg oil.

Another embodiment comprises cyclosporin A, Armotan AL-69-66®, and Nutmeg oil.

Another embodiment comprises cyclosporin A, Cerasynt 840®, and Nutmeg oil.

Another embodiment comprises cyclosporin A, Cerasynt 945®, and Nutmeg oil.

Another embodiment comprises cyclosporin A, Crystal Inhibitor No. 5®, and Nutmeg oil.

Another embodiment comprises cyclosporin A, DeThox Acid L-9®, and Nutmeg oil.

Another embodiment comprises cyclosporin A, DeThox Acid S-8®, and Nutmeg oil.

Another embodiment comprises cyclosporin A, Ethofat 242/25®, and Nutmeg oil.

Another embodiment comprises cyclosporin A, Hydropalat 65®, and Nutmeg oil.

Another embodiment comprises cyclosporin A, Lipo EGMS®, and Nutmeg oil.

Another embodiment comprises cyclosporin A, Lipopeg 2 DL®, and Nutmeg oil.

Another embodiment comprises cyclosporin A, Lipopeg 4 DL®, and Nutmeg oil.

Another embodiment comprises cyclosporin A, Lipopeg 4-L®, and Nutmeg oil.

Another embodiment comprises cyclosporin A, Lipopeg 39-S®, and Nutmeg oil.

Another embodiment comprises cyclosporin A, Lipopeg 4-S®, and Nutmeg oil.

Another embodiment comprises cyclosporin A, Lipopeg 10-S®, and Nutmeg oil.

Another embodiment comprises cyclosporin A, Lipopeg 100-S®, and Nutmeg oil.

Another embodiment comprises cyclosporin A, Lipopeg 6000 DS®, and Nutmeg oil.

Another embodiment comprises cyclosporin A, Lumulse 40-L®, and Nutmeg oil.

Another embodiment comprises cyclosporin A, Lumulse 40-S®, and Nutmeg oil.

Another embodiment comprises cyclosporin A, Lumulse 42-L®, and Nutmeg oil.

Another embodiment comprises cyclosporin A, Lumulse 42-S®, and Nutmeg oil.

Another embodiment comprises cyclosporin A, Lumulse 100-S®, and Nutmeg oil.

Another embodiment comprises cyclosporin A, Lumulse 602-S®, and Nutmeg oil.

Another embodiment comprises cyclosporin A, Magrabar PGE-20-0®, and Nutmeg oil.

Another embodiment comprises cyclosporin A, Magrabar PGE-20L®, and Nutmeg oil.

Another embodiment comprises cyclosporin A, Magrabar PGE-20T®, and Nutmeg oil.

Another embodiment comprises cyclosporin A, Magrabar PGE-22-0®, and Nutmeg oil.

Another embodiment comprises cyclosporin A, Magrabar PGE-22L®, and Nutmeg oil.

Another embodiment comprises cyclosporin A, Magrabar PGE-22T®, and Nutmeg oil.

Another embodiment comprises cyclosporin A, Magrabar PGE-40-0®, and Nutmeg oil.

Another embodiment comprises cyclosporin A, Magrabar PGE-40L®, and Nutmeg oil.

Another embodiment comprises cyclosporin A, Magrabar PGE-40T®, and Nutmeg oil.

Another embodiment comprises cyclosporin A, Magrabar PGE-42-0®, and Nutmeg oil.

Another embodiment comprises cyclosporin A, Magrabar PGE-42L®, and Nutmeg oil.

Another embodiment comprises cyclosporin A, Magrabar PGE-42T®, and Nutmeg oil.

Another embodiment comprises cyclosporin A, Magrabar PGE-60-0®, and Nutmeg oil.

Another embodiment comprises cyclosporin A, Magrabar PGE-60L®, and Nutmeg oil.

Another embodiment comprises cyclosporin A, Magrabar PGE-60T®, and Nutmeg oil.

Another embodiment comprises cyclosporin A, Magrabar PGE-62-0®, and Nutmeg oil.

Another embodiment comprises cyclosporin A, Magrabar PGE-62L®, and Nutmeg oil.

Another embodiment comprises cyclosporin A, Magrabar PGE-62T®, and Nutmeg oil.

Another embodiment comprises cyclosporin A, Mapeg S-40K®, and Nutmeg oil.

Another embodiment comprises cyclosporin A, Marlowet OTS®, and Nutmeg oil.

Another embodiment comprises cyclosporin A, Naturechem PGR®, and Nutmeg oil.

Another embodiment comprises cyclosporin A, PG No. 4®, and Nutmeg oil.

Another embodiment comprises cyclosporin A, Renex 20®, and Nutmeg oil.

Another embodiment comprises cyclosporin A, Ritox 52®, and Nutmeg oil.

Another embodiment comprises cyclosporin A, Ritox 53®, and Nutmeg oil.

Another embodiment comprises cyclosporin A, Ritox 59®, and Nutmeg oil.

Another embodiment comprises cyclosporin A, Surfax 8916/A®, and Nutmeg oil.

Another embodiment comprises cyclosporin A, Tego Acid S 40 P®, and Nutmeg oil.

Another embodiment comprises cyclosporin A, Tego Acid S 100 P®, and Nutmeg oil.

Another embodiment comprises cyclosporin A, Tween 20®, and Nutmeg oil.

Another embodiment comprises cyclosporin A, Volpo 131®, and Nutmeg oil.

Another embodiment comprises cyclosporin A, an Ethoxylated Fatty Ester or Oil, and Nutmeg oil.

Another embodiment comprises cyclosporin A, Acconon 6-C10®, and Nutmeg oil.

Another embodiment comprises cyclosporin A, Acconon CC-6®, and Nutmeg oil.

Another embodiment comprises cyclosporin A, Acconon CO-7®, and Nutmeg oil.

Another embodiment comprises cyclosporin A, Aldosperse 40/60 FG®, and Nutmeg oil.

Another embodiment comprises cyclosporin A, Aldosperse ML-23®, and Nutmeg oil.

Another embodiment comprises cyclosporin A, Aldosperse MS-20 FG®, and Nutmeg oil.

Another embodiment comprises cyclosporin A, Alkamuls EL-620®, and Nutmeg oil.

Another embodiment comprises cyclosporin A, Alkamuls EL-719®, and Nutmeg oil.

Another embodiment comprises cyclosporin A, Alkamuls EL-985®, and Nutmeg oil.

Another embodiment comprises cyclosporin A, Arlatone G®, and Nutmeg oil.

Another embodiment comprises cyclosporin A, Arlatone T®, and Nutmeg oil.

Another embodiment comprises cyclosporin A, Atlas G-1045A®, and Nutmeg oil.

Another embodiment comprises cyclosporin A, Atlas G-1086®, and Nutmeg oil.

Another embodiment comprises cyclosporin A, Atlas G-1087®, and Nutmeg oil.

Another embodiment comprises cyclosporin A, Atlas G-1089®, and Nutmeg oil.

Another embodiment comprises cyclosporin A, Atlas G-1096®, and Nutmeg oil.

Another embodiment comprises cyclosporin A, Atlas G-1292®, and Nutmeg oil.

Another embodiment comprises cyclosporin A, Atlas G-1293®, and Nutmeg oil.

Another embodiment comprises cyclosporin A, Atlas G-1300®, and Nutmeg oil.

Another embodiment comprises cyclosporin A, Atlas G-7076®, and Nutmeg oil.

Another embodiment comprises cyclosporin A, Capmul EMG®, and Nutmeg oil.

Another embodiment comprises cyclosporin A, Chemonic CO-40®, and Nutmeg oil.

Another embodiment comprises cyclosporin A, Chemonic LI-3®, and Nutmeg oil.

Another embodiment comprises cyclosporin A, Chemonic LI-7®, and Nutmeg oil.

Another embodiment comprises cyclosporin A, Cirrasol GM®, and Nutmeg oil.

Another embodiment comprises cyclosporin A, Cremophor CO 40®, and Nutmeg oil.

Another embodiment comprises cyclosporin A, Cremophor CO 410®, and Nutmeg oil.

Another embodiment comprises cyclosporin A, Cremophor EL®, and Nutmeg oil.

Another embodiment comprises cyclosporin A, Cremophor GC7®, and Nutmeg oil.

Another embodiment comprises cyclosporin A, Cremophor RH-40®, and Nutmeg oil.

Another embodiment comprises cyclosporin A, Crovol A-40®, and Nutmeg oil.

Another embodiment comprises cyclosporin A, Crovol A-70®, and Nutmeg oil.

Another embodiment comprises cyclosporin A, Crovol M-70®, and Nutmeg oil.

Another embodiment comprises cyclosporin A, Crovol PK-70®, and Nutmeg oil.

Another embodiment comprises cyclosporin A, Cutina E-24®, and Nutmeg oil.

Another embodiment comprises cyclosporin A, Dacospin 12-R®, and Nutmeg oil.

Another embodiment comprises cyclosporin A, Dehymuls HRE-7®, and Nutmeg oil.

Another embodiment comprises cyclosporin A, DeSonic 30C®, and Nutmeg oil.

Another embodiment comprises cyclosporin A, DeSonic 36C®, and Nutmeg oil.

Another embodiment comprises cyclosporin A, DeSonic 40C®, and Nutmeg oil.

Another embodiment comprises cyclosporin A, Durfax 60®, and Nutmeg oil.

Another embodiment comprises cyclosporin A, Durfax 65®, and Nutmeg oil.

Another embodiment comprises cyclosporin A, Durfax 80®, and Nutmeg oil.

Another embodiment comprises cyclosporin A, Durfax EOM®, and Nutmeg oil.

Another embodiment comprises cyclosporin A, Eccoterge NF-2®, and Nutmeg oil.

Another embodiment comprises cyclosporin A, Emulpon CO-360®, and Nutmeg oil.

Another embodiment comprises cyclosporin A, Emulpon CO-550®, and Nutmeg oil.

Another embodiment comprises cyclosporin A, Emulsogen EL®, and Nutmeg oil.

Another embodiment comprises cyclosporin A, Emulsogen HCO 040®, and Nutmeg oil.

Another embodiment comprises cyclosporin A, Emulsogen HCO 060®, and Nutmeg oil.

Another embodiment comprises cyclosporin A, Emulsynt 1055®, and Nutmeg oil.

Another embodiment comprises cyclosporin A, Ethox 3095®, and Nutmeg oil.

Another embodiment comprises cyclosporin A, Eumulgin RO-40®, and Nutmeg oil.

Another embodiment comprises cyclosporin A, Genapol G-260®, and Nutmeg oil.

Another embodiment comprises cyclosporin A, Glycosperse L-20®, and Nutmeg oil.

Another embodiment comprises cyclosporin A, Glycosperse O-5®, and Nutmeg oil.

Another embodiment comprises cyclosporin A, Glycosperse O-20®, and Nutmeg oil.

Another embodiment comprises cyclosporin A, Glycosperse O-20 FG®, and Nutmeg oil.

Another embodiment comprises cyclosporin A, Glycosperse S-20®, and Nutmeg oil.

Another embodiment comprises cyclosporin A, Glycosperse S-20 FG®, and Nutmeg oil.

Another embodiment comprises cyclosporin A, Glycosperse TS-20®, and Nutmeg oil.

Another embodiment comprises cyclosporin A, Glycosperse TS-20 FG®, and Nutmeg oil.

Another embodiment comprises cyclosporin A, Hetan SL®, and Nutmeg oil.

Another embodiment comprises cyclosporin A, Hetan SO®, and Nutmeg oil.

Another embodiment comprises cyclosporin A, Hetan SS®, and Nutmeg oil.

Another embodiment comprises cyclosporin A, Hetoxide C-2®, and Nutmeg oil.

Another embodiment comprises cyclosporin A, Hetoxide C-9®, and Nutmeg oil.

Another embodiment comprises cyclosporin A, Hetoxide C-15®, and Nutmeg oil.

Another embodiment comprises cyclosporin A, Hetoxide C-25®, and Nutmeg oil.

Another embodiment comprises cyclosporin A, Hetoxide C-40®, and Nutmeg oil.

Another embodiment comprises cyclosporin A, Hetoxide C-200®, and Nutmeg oil.

Another embodiment comprises cyclosporin A, Hetoxide C-200-50%®, and Nutmeg oil.

Another embodiment comprises cyclosporin A, Hetoxide GC-30®, and Nutmeg oil.

Another embodiment comprises cyclosporin A, Hetoxide HC-60®, and Nutmeg oil.

Another embodiment comprises cyclosporin A, Ice No. 2®, and Nutmeg oil.

Another embodiment comprises cyclosporin A, Incrocas 30/40®, and Nutmeg oil.

Another embodiment comprises cyclosporin A, Lexol EC®, and Nutmeg oil.

Another embodiment comprises cyclosporin A, Lexol EO®, and Nutmeg oil.

Another embodiment comprises cyclosporin A, Lipocol HCO-40®, and Nutmeg oil.

Another embodiment comprises cyclosporin A, Lipocol HCO-60®, and Nutmeg oil.

Another embodiment comprises cyclosporin A, Lipocol O-3 Specials, and Nutmeg oil.

Another embodiment comprises cyclosporin A, Lipopeg 2-L®, and Nutmeg oil.

Another embodiment comprises cyclosporin A, Lipopeg 4-DO®, and Nutmeg oil.

Another embodiment comprises cyclosporin A, Lipopeg 4-DS®, and Nutmeg oil.

Another embodiment comprises cyclosporin A, Lipovol GTB®, and Nutmeg oil.

Another embodiment comprises cyclosporin A, Lonzest SML-20®, and Nutmeg oil.

Another embodiment comprises cyclosporin A, Lonzest SMO-20®, and Nutmeg oil.

Another embodiment comprises cyclosporin A, Lonzest SMS-20®, and Nutmeg oil.

Another embodiment comprises cyclosporin A, Lonzest STO-20®, and Nutmeg oil.

Another embodiment comprises cyclosporin A, Lonzest STS-20®, and Nutmeg oil.

Another embodiment comprises cyclosporin A, Lumulse GR-40®, and Nutmeg oil.

Another embodiment comprises cyclosporin A, Lumulse GRH-40®, and Nutmeg oil.

Another embodiment comprises cyclosporin A, Lumulse POE (7) GML®, and Nutmeg oil.

Another embodiment comprises cyclosporin A, Lumulse POE (12) Glyc®, and Nutmeg oil.

Another embodiment comprises cyclosporin A, Lumulse POE (40) MS KP®, and Nutmeg oil.

Another embodiment comprises cyclosporin A, Marlowet 4750®, and Nutmeg oil.

Another embodiment comprises cyclosporin A, Marlowet LVS®, and Nutmeg oil.

Another embodiment comprises cyclosporin A, Marlowet R 11®, and Nutmeg oil.

Another embodiment comprises cyclosporin A, Marlowet R 40®, and Nutmeg oil.

Another embodiment comprises cyclosporin A, Mazol 80 MGK®, and Nutmeg oil.

Another embodiment comprises cyclosporin A, Nonionic Emulsifier T-9®, and Nutmeg oil.

Another embodiment comprises cyclosporin A, Oronal LCG®, and Nutmeg oil.

Another embodiment comprises cyclosporin A, Polyderm PPI-CO-200®, and Nutmeg oil.

Another embodiment comprises cyclosporin A, Polyderm PPI-CO-40®, and Nutmeg oil.

Another embodiment comprises cyclosporin A, Rewoderm LI 520-70®, and Nutmeg oil.

Another embodiment comprises cyclosporin A, Ritapeg 150 DS®, and Nutmeg oil.

Another embodiment comprises cyclosporin A, Softigen 767®, and Nutmeg oil.

Another embodiment comprises cyclosporin A, Surfactol 318®, and Nutmeg oil.

Another embodiment comprises cyclosporin A, Surfactol 365®, and Nutmeg oil.

Another embodiment comprises cyclosporin A, Syn Lube 107®, and Nutmeg oil.

Another embodiment comprises cyclosporin A, Syn Lube 728®, and Nutmeg oil.

Another embodiment comprises cyclosporin A, Syn Lube 1632H®, and Nutmeg oil.

Another embodiment comprises cyclosporin A, Syn Lube 6277-A®, and Nutmeg oil.

Another embodiment comprises cyclosporin A, T-Det C-20®, and Nutmeg oil.

Another embodiment comprises cyclosporin A, T-Det C-40®, and Nutmeg oil.

Another embodiment comprises cyclosporin A, Tally 100 Plus®, and Nutmeg oil.

Another embodiment comprises cyclosporin A, Uniperol EL®, and Nutmeg oil.

Another embodiment comprises cyclosporin A, a Fatty Ester, and Nutmeg oil.

Another embodiment comprises cyclosporin A, Actralube-Syn 147®, and Nutmeg oil.

Another embodiment comprises cyclosporin A, Atlas G-1556®, and Nutmeg oil.

Another embodiment comprises cyclosporin A, Atlas G-1564®, and Nutmeg oil.

Another embodiment comprises cyclosporin A, Atlasol Base Oil S®, and Nutmeg oil.

Another embodiment comprises cyclosporin A, Base ML®, and Nutmeg oil.

Another embodiment comprises cyclosporin A, Base MT®, and Nutmeg oil.

Another embodiment comprises cyclosporin A, Cerasynt 303®, and Nutmeg oil.

Another embodiment comprises cyclosporin A, Dermol 1012®, and Nutmeg oil.

Another embodiment comprises cyclosporin A, Kemester 4000®, and Nutmeg oil.

Another embodiment comprises cyclosporin A, Lactipol S®, and Nutmeg oil.

Another embodiment comprises cyclosporin A, Magrabar PGO®, and Nutmeg oil.

Another embodiment comprises cyclosporin A, Mayco Base BFO®, and Nutmeg oil.

Another embodiment comprises cyclosporin A, Methyl Linoleate®, and Nutmeg oil.

Another embodiment comprises cyclosporin A, Pationic 122A®, and Nutmeg oil.

Another embodiment comprises cyclosporin A, Pationic 138C®, and Nutmeg oil.

Another embodiment comprises cyclosporin A, Pationic CSL®, and Nutmeg oil.

Another embodiment comprises cyclosporin A, Pationic ISL®, and Nutmeg oil.

Another embodiment comprises cyclosporin A, Pationic SBL®, and Nutmeg oil.

Another embodiment comprises cyclosporin A, Pationic SSL®, and Nutmeg oil.

Another embodiment comprises cyclosporin A, Ritasol®, and Nutmeg oil.

Another embodiment comprises cyclosporin A, Tego Alkanol CS 20®, and Nutmeg oil.

Another embodiment comprises cyclosporin A, Tego Alkanol L23 P®, and Nutmeg oil.

Another embodiment comprises cyclosporin A, Tego Alkanol S2®, and Nutmeg oil.

Another embodiment comprises cyclosporin A, Tego Alkanol S20 P®, and Nutmeg oil.

Another embodiment comprises cyclosporin A, Triemulsifier 600 MS®, and Nutmeg oil.

Another embodiment comprises cyclosporin A, a Glycerol Esters, and Nutmeg oil.

Another embodiment comprises cyclosporin A, Agro #9 Wint SBO®, and Nutmeg oil.

Another embodiment comprises cyclosporin A, Ahcovel Base 700®, and Nutmeg oil.

Another embodiment comprises cyclosporin A, Aldo HMS FG®, and Nutmeg oil.

Another embodiment comprises cyclosporin A, Aldo MLD®, and Nutmeg oil.

Another embodiment comprises cyclosporin A, Aldo MLD FG®, and Nutmeg oil.

Another embodiment comprises cyclosporin A, Aldo MO FG®, and Nutmeg oil.

Another embodiment comprises cyclosporin A, Aldo MS®, and Nutmeg oil.

Another embodiment comprises cyclosporin A, Aldo MS FG®, and Nutmeg oil.

Another embodiment comprises cyclosporin A, Aldo MS LG FG®, and Nutmeg oil.

Another embodiment comprises cyclosporin A, Aldo MSD®, and Nutmeg oil.

Another embodiment comprises cyclosporin A, Aldo MSD FG®, and Nutmeg oil.

Another embodiment comprises cyclosporin A, Aldosperse O-20 FG®, and Nutmeg oil.

Another embodiment comprises cyclosporin A, Aldosperse TS-20 FG®, and Nutmeg oil.

Another embodiment comprises cyclosporin A, Aldosperse TS-40 FG®, and Nutmeg oil.

Another embodiment comprises cyclosporin A, Arlacel 165®, and Nutmeg oil.

Another embodiment comprises cyclosporin A, Arlacel 186®, and Nutmeg oil.

Another embodiment comprises cyclosporin A, Capmul GMO®, and Nutmeg oil.

Another embodiment comprises cyclosporin A, Capmul GMS®, and Nutmeg oil.

Another embodiment comprises cyclosporin A, Caprol 3GO®, and Nutmeg oil.

Another embodiment comprises cyclosporin A, Caprol 3GVS®, and Nutmeg oil.

Another embodiment comprises cyclosporin A, Caprol 6G2S®, and Nutmeg oil.

Another embodiment comprises cyclosporin A, Caprol 10G40®, and Nutmeg oil.

Another embodiment comprises cyclosporin A, Caprol 10G100®, and Nutmeg oil.

Another embodiment comprises cyclosporin A, Caprol ET®, and Nutmeg oil.

Another embodiment comprises cyclosporin A, Caprol PGE860®, and Nutmeg oil.

Another embodiment comprises cyclosporin A, Cerasynt 945®, and Nutmeg oil.

Another embodiment comprises cyclosporin A, Cerasynt GMS®, and Nutmeg oil.

Another embodiment comprises cyclosporin A, Cerasynt Q®, and Nutmeg oil.

Another embodiment comprises cyclosporin A, Cerasynt SD®, and Nutmeg oil.

Another embodiment comprises cyclosporin A, Cerasynt WM®, and Nutmeg oil.

Another embodiment comprises cyclosporin A, Chemsperse 14®, and Nutmeg oil.

Another embodiment comprises cyclosporin A, Cremophor GO-32®, and Nutmeg oil.

Another embodiment comprises cyclosporin A, Cremophor GS11®, and Nutmeg oil.

Another embodiment comprises cyclosporin A, Cremophor GS-32®, and Nutmeg oil.

Another embodiment comprises cyclosporin A, Cutina KD-16®, and Nutmeg oil.

Another embodiment comprises cyclosporin A, Dehymuls PGPH®, and Nutmeg oil.

Another embodiment comprises cyclosporin A, Dermol DGDIS®, and Nutmeg oil.

Another embodiment comprises cyclosporin A, Dermol DGMIS®, and Nutmeg oil.

Another embodiment comprises cyclosporin A, Dermol G-76®, and Nutmeg oil.

Another embodiment comprises cyclosporin A, Dermol G-7DI®, and Nutmeg oil.

Another embodiment comprises cyclosporin A, Dermol NGDI®, and Nutmeg oil.

Another embodiment comprises cyclosporin A, Dermolan GLH®, and Nutmeg oil.

Another embodiment comprises cyclosporin A, Drewmulse GMO®, and Nutmeg oil.

Another embodiment comprises cyclosporin A, Drewpol 3-5-M®, and Nutmeg oil.

Another embodiment comprises cyclosporin A, Durlac 100 W®, and Nutmeg oil.

Another embodiment comprises cyclosporin A, Dur-Lo®, and Nutmeg oil.

Another embodiment comprises cyclosporin A, Dynasan 118®, and Nutmeg oil.

Another embodiment comprises cyclosporin A, EC-25®, and Nutmeg oil.

Another embodiment comprises cyclosporin A, EM 40®, and Nutmeg oil.

Another embodiment comprises cyclosporin A, Emerest 2400®, and Nutmeg oil.

Another embodiment comprises cyclosporin A, Emerest 2452®, and Nutmeg oil.

Another embodiment comprises cyclosporin A, Empilan G-26®, and Nutmeg oil.

Another embodiment comprises cyclosporin A, Genapol TSM®, and Nutmeg oil.

Another embodiment comprises cyclosporin A, Hostacerin DGI®, and Nutmeg oil.

Another embodiment comprises cyclosporin A, Hostacerin DGL®, and Nutmeg oil.

Another embodiment comprises cyclosporin A, Hostacerin DGMS®, and Nutmeg oil.

Another embodiment comprises cyclosporin A, Hostacerin DGSB®, and Nutmeg oil.

Another embodiment comprises cyclosporin A, Ice No. 2®, and Nutmeg oil.

Another embodiment comprises cyclosporin A, Imwitor 742®, and Nutmeg oil.

Another embodiment comprises cyclosporin A, Imwitor 780 K®, and Nutmeg oil.

Another embodiment comprises cyclosporin A, Imwitor 960 Flakes®, and Nutmeg oil.

Another embodiment comprises cyclosporin A, Isolan GI 34®, and Nutmeg oil.

Another embodiment comprises cyclosporin A, Isolan GO 33®, and Nutmeg oil.

Another embodiment comprises cyclosporin A, Kemester 1000®, and Nutmeg oil.

Another embodiment comprises cyclosporin A, Kemester 2000®, and Nutmeg oil.

Another embodiment comprises cyclosporin A, Kemester 2000®, and Nutmeg oil.

Another embodiment comprises cyclosporin A, Kemester 6000SE®, and Nutmeg oil.

Another embodiment comprises cyclosporin A, Lamecreme DGE 18®, and Nutmeg oil.

Another embodiment comprises cyclosporin A, Lexemul 515®, and Nutmeg oil.

Another embodiment comprises cyclosporin A, Lexemul 561®, and Nutmeg oil.

Another embodiment comprises cyclosporin A, Lexemul AR®, and Nutmeg oil.

Another embodiment comprises cyclosporin A, Lexemul AS®, and Nutmeg oil.

Another embodiment comprises cyclosporin A, Lexemul GDL®, and Nutmeg oil.

Another embodiment comprises cyclosporin A, Lexemul T®, and Nutmeg oil.

Another embodiment comprises cyclosporin A, Lipomulse 165®, and Nutmeg oil.

Another embodiment comprises cyclosporin A, Lumulse GML K®, and Nutmeg oil.

Another embodiment comprises cyclosporin A, Lumulse GMO K®, and Nutmeg oil.

Another embodiment comprises cyclosporin A, Lumulse GMR K®, and Nutmeg oil.

Another embodiment comprises cyclosporin A, Lumulse GMT K®, and Nutmeg oil.

Another embodiment comprises cyclosporin A, Magrabar GMC®, and Nutmeg oil.

Another embodiment comprises cyclosporin A, Magrabar GMO-CK®, and Nutmeg oil.

Another embodiment comprises cyclosporin A, Magrabar MDG-5050®, and Nutmeg oil.

Another embodiment comprises cyclosporin A, Magrabar PGO-315®, and Nutmeg oil.

Another embodiment comprises cyclosporin A, Magrabar PGO-1010®, and Nutmeg oil.

Another embodiment comprises cyclosporin A, Mazol 300K®, and Nutmeg oil.

Another embodiment comprises cyclosporin A, Mazol GMO-K®, and Nutmeg oil.

Another embodiment comprises cyclosporin A, Mazol GMS-K®, and Nutmeg oil.

Another embodiment comprises cyclosporin A, Mazol PG031-K®, and Nutmeg oil.

Another embodiment comprises cyclosporin A, Miglyol 812®, and Nutmeg oil.

Another embodiment comprises cyclosporin A, Norfox 165C®, and Nutmeg oil.

Another embodiment comprises cyclosporin A, Schercemol GMIS®, and Nutmeg oil.

Another embodiment comprises cyclosporin A, Tegin®, and Nutmeg oil.

Another embodiment comprises cyclosporin A, Tegin 4100 Pellets®, and Nutmeg oil.

Another embodiment comprises cyclosporin A, Tegin M Pellets®, and Nutmeg oil.

Another embodiment comprises cyclosporin A, Tegin OV®, and Nutmeg oil.

Another embodiment comprises cyclosporin A, Teginacid H®, and Nutmeg oil.

Another embodiment comprises cyclosporin A, Tego Cosmo P813®, and Nutmeg oil.

Another embodiment comprises cyclosporin A, Wickenol 535®, and Nutmeg oil.

Another embodiment comprises cyclosporin A, Witconol 14®, and Nutmeg oil.

Another embodiment comprises cyclosporin A, Witconol 14®, and Nutmeg oil.

Another embodiment comprises cyclosporin A, Witconol 14F®, and Nutmeg oil.

Another embodiment comprises cyclosporin A, Witconol 18L®, and Nutmeg oil.

Another embodiment comprises cyclosporin A, Witconol GOT®, and Nutmeg oil.

Another embodiment comprises cyclosporin A, Witconol MST®, and Nutmeg oil.

Another embodiment comprises cyclosporin A, Witconol RHT®, and Nutmeg oil.

Another embodiment comprises cyclosporin A, a Glycol Ester, and Nutmeg oil.

Another embodiment comprises cyclosporin A, Alkamuls 600 DO®, and Nutmeg oil.

Another embodiment comprises cyclosporin A, Alkamuls SEG®, and Nutmeg oil.

Another embodiment comprises cyclosporin A, Atlas EM-2®, and Nutmeg oil.

Another embodiment comprises cyclosporin A, Cerasynt IP®, and Nutmeg oil.

Another embodiment comprises cyclosporin A, Cerasynt M®, and Nutmeg oil.

Another embodiment comprises cyclosporin A, Cerasynt MN®, and Nutmeg oil.

Another embodiment comprises cyclosporin A, Cerasynt PA®, and Nutmeg oil.

Another embodiment comprises cyclosporin A, Chemsperse EGDS®, and Nutmeg oil.

Another embodiment comprises cyclosporin A, Chemsperse EGMS®, and Nutmeg oil.

Another embodiment comprises cyclosporin A, Colonial Monolaurin®, and Nutmeg oil.

Another embodiment comprises cyclosporin A, DeMuls SGE-95®, and Nutmeg oil.

Another embodiment comprises cyclosporin A, Eccoterge 200®, and Nutmeg oil.

Another embodiment comprises cyclosporin A, Emerest 2380®, and Nutmeg oil.

Another embodiment comprises cyclosporin A, Ethox 2610®, and Nutmeg oil.

Another embodiment comprises cyclosporin A, Ethox DO-9®, and Nutmeg oil.

Another embodiment comprises cyclosporin A, Ethox DO-14®, and Nutmeg oil.

Another embodiment comprises cyclosporin A, Ethox SO-9®, and Nutmeg oil.

Another embodiment comprises cyclosporin A, Fizul MD-318®, and Nutmeg oil.

Another embodiment comprises cyclosporin A, Genapol EGDS-VHP®, and Nutmeg oil.

Another embodiment comprises cyclosporin A, Genapol TS Powder®, and Nutmeg oil.

Another embodiment comprises cyclosporin A, Hostacerin WO®, and Nutmeg oil.

Another embodiment comprises cyclosporin A, Inversol 140®, and Nutmeg oil.

Another embodiment comprises cyclosporin A, Kemester 104®, and Nutmeg oil.

Another embodiment comprises cyclosporin A, Kemester 205®, and Nutmeg oil.

Another embodiment comprises cyclosporin A, Kemester 226®, and Nutmeg oil.

Another embodiment comprises cyclosporin A, Kemester 5221SE®, and Nutmeg oil.

Another embodiment comprises cyclosporin A, Kemester EGDS®, and Nutmeg oil.

Another embodiment comprises cyclosporin A, Lexemul EGDS®, and Nutmeg oil.

Another embodiment comprises cyclosporin A, Lexemul EGMS®, and Nutmeg oil.

Another embodiment comprises cyclosporin A, Lexemul P®, and Nutmeg oil.

Another embodiment comprises cyclosporin A, Lipo DGLS®, and Nutmeg oil.

Another embodiment comprises cyclosporin A, Lipo EGDS®, and Nutmeg oil.

Another embodiment comprises cyclosporin A, Lipo PGMS®, and Nutmeg oil.

Another embodiment comprises cyclosporin A, Liposorb S-4®, and Nutmeg oil.

Another embodiment comprises cyclosporin A, Liposorb TO-20®, and Nutmeg oil.

Another embodiment comprises cyclosporin A, Lumulse PGO®, and Nutmeg oil.

Another embodiment comprises cyclosporin A, Mackester EGDS®, and Nutmeg oil.

Another embodiment comprises cyclosporin A, Mackester EGMS®, and Nutmeg oil.

Another embodiment comprises cyclosporin A, Mackester GSTP®, and Nutmeg oil.

Another embodiment comprises cyclosporin A, Mackester Series®, and Nutmeg oil.

Another embodiment comprises cyclosporin A, Magrabar PDG-50®, and Nutmeg oil.

Another embodiment comprises cyclosporin A, Mapeg 6000 DS®, and Nutmeg oil.

Another embodiment comprises cyclosporin A, Marlowet 4702®, and Nutmeg oil.

Another embodiment comprises cyclosporin A, Monalube 305®, and Nutmeg oil.

Another embodiment comprises cyclosporin A, Monalube 310®, and Nutmeg oil.

Another embodiment comprises cyclosporin A, Monalube 315®, and Nutmeg oil.

Another embodiment comprises cyclosporin A, Monalube 320®, and Nutmeg oil.

Another embodiment comprises cyclosporin A, Monalube 325®, and Nutmeg oil.

Another embodiment comprises cyclosporin A, Monalube 330®, and Nutmeg oil.

Another embodiment comprises cyclosporin A, Naturechem PGHS®, and Nutmeg oil.

Another embodiment comprises cyclosporin A, Polycastorol PLO-840®, and Nutmeg oil.

Another embodiment comprises cyclosporin A, Polytex 10M®, and Nutmeg oil.

Another embodiment comprises cyclosporin A, Ritasynt IP®, and Nutmeg oil.

Another embodiment comprises cyclosporin A, Ross Chem PEG 600 DT®, and Nutmeg oil.

Another embodiment comprises cyclosporin A, Schercemol PGMS®, and Nutmeg oil.

Another embodiment comprises cyclosporin A, Sponto H-44C®, and Nutmeg oil.

Another embodiment comprises cyclosporin A, Tegin G®, and Nutmeg oil.

Another embodiment comprises cyclosporin A, Witbreak DGE-182®, and Nutmeg oil.

Another embodiment comprises cyclosporin A, Witbreak DGE-182®, and Nutmeg oil.

Another embodiment comprises cyclosporin A, Witbreak DRA-21®, and Nutmeg oil.

Another embodiment comprises cyclosporin A, Witbreak DRA-21®, and Nutmeg oil.

Another embodiment comprises cyclosporin A, Witbreak DRA-50®, and Nutmeg oil.

Another embodiment comprises cyclosporin A, Witbreak DRA-50®, and Nutmeg oil.

Another embodiment comprises cyclosporin A, Witconol F26-46®, and Nutmeg oil.

Another embodiment comprises cyclosporin A, Witconol H-32®, and Nutmeg oil.

Another embodiment comprises cyclosporin A, Witconol H-33®, and Nutmeg oil.

Another embodiment comprises cyclosporin A, Witconol H-35A®, and Nutmeg oil.

Another embodiment comprises cyclosporin A, Witconol RHP®, and Nutmeg oil.

Another embodiment comprises cyclosporin A, a Lanolin-based Derivative, and Nutmeg oil.

Another embodiment comprises cyclosporin A, Amerchol CAB®, and Nutmeg oil.

Another embodiment comprises cyclosporin A, Amerchol L-101®, and Nutmeg oil.

Another embodiment comprises cyclosporin A, Amerlate LFA-LO®, and Nutmeg oil.

Another embodiment comprises cyclosporin A, Amerlate P®, and Nutmeg oil.

Another embodiment comprises cyclosporin A, Barre Common Degras®, and Nutmeg oil.

Another embodiment comprises cyclosporin A, Cholesterol NF®, and Nutmeg oil.

Another embodiment comprises cyclosporin A, Crodalan AWS®, and Nutmeg oil.

Another embodiment comprises cyclosporin A, Crodalan LA®, and Nutmeg oil.

Another embodiment comprises cyclosporin A, Emery 1650®, and Nutmeg oil.

Another embodiment comprises cyclosporin A, Emery 1650®, and Nutmeg oil.

Another embodiment comprises cyclosporin A, Emery 1740®, and Nutmeg oil.

Another embodiment comprises cyclosporin A, Emery 1740®, and Nutmeg oil.

Another embodiment comprises cyclosporin A, Forlan 500®, and Nutmeg oil.

Another embodiment comprises cyclosporin A, Forlan L®, and Nutmeg oil.

Another embodiment comprises cyclosporin A, Laneto 50®, and Nutmeg oil.

Another embodiment comprises cyclosporin A, Laneto 100®, and Nutmeg oil.

Another embodiment comprises cyclosporin A, Laneto AWS®, and Nutmeg oil.

Another embodiment comprises cyclosporin A, Lanfrax 1776®, and Nutmeg oil.

Another embodiment comprises cyclosporin A, Lanfrax 1776®, and Nutmeg oil.

Another embodiment comprises cyclosporin A, Lanogel 21®, and Nutmeg oil.

Another embodiment comprises cyclosporin A, Lipolan®, and Nutmeg oil.

Another embodiment comprises cyclosporin A, Lipolan 31®, and Nutmeg oil.

Another embodiment comprises cyclosporin A, OHlan®, and Nutmeg oil.

Another embodiment comprises cyclosporin A, Polychol 5®, and Nutmeg oil.

Another embodiment comprises cyclosporin A, Polychol 15®, and Nutmeg oil.

Another embodiment comprises cyclosporin A, Ritacetyl®, and Nutmeg oil.

Another embodiment comprises cyclosporin A, Ritachol®, and Nutmeg oil.

Another embodiment comprises cyclosporin A, Ritahydrox®, and Nutmeg oil.

Another embodiment comprises cyclosporin A, Ritalafa®, and Nutmeg oil.

Another embodiment comprises cyclosporin A, Ritalan®, and Nutmeg oil.

Another embodiment comprises cyclosporin A, Ritalan AWS®, and Nutmeg oil.

Another embodiment comprises cyclosporin A, Ritalan C®, and Nutmeg oil.

Another embodiment comprises cyclosporin A, Ritawax®, and Nutmeg oil.

Another embodiment comprises cyclosporin A, Ritawax AEO®, and Nutmeg oil.

Another embodiment comprises cyclosporin A, Ritawax ALA®, and Nutmeg oil.

Another embodiment comprises cyclosporin A, Solan/Solan 50/Super Solan®, and Nutmeg oil.

Another embodiment comprises cyclosporin A, Super Hartolan/Hartolan®, and Nutmeg oil.

Another embodiment comprises cyclosporin A, Supersat AWS-4®, and Nutmeg oil.

Another embodiment comprises cyclosporin A, Supersat AWS-24®, and Nutmeg oil.

Another embodiment comprises cyclosporin A, a Lecithin or Lecithin Derivative, and Nutmeg oil.

Another embodiment comprises cyclosporin A, Alcolec®, and Nutmeg oil.

Another embodiment comprises cyclosporin A, Lecithin®, and Nutmeg oil.

Another embodiment comprises cyclosporin A, Lexin K®, and Nutmeg oil.

Another embodiment comprises cyclosporin A, Natipide®, and Nutmeg oil.

Another embodiment comprises cyclosporin A, a Lignin or Lignin Derivative, and Nutmeg oil.

Another embodiment comprises cyclosporin A, Diwatex XP 9®, and Nutmeg oil.

Another embodiment comprises cyclosporin A, Dynasperse LCD®, and Nutmeg oil.

Another embodiment comprises cyclosporin A, Indulin SAL®, and Nutmeg oil.

Another embodiment comprises cyclosporin A, Indulin W-1®, and Nutmeg oil.

Another embodiment comprises cyclosporin A, Indulin W-5®, and Nutmeg oil.

Another embodiment comprises cyclosporin A, Lignosol FTA®, and Nutmeg oil.

Another embodiment comprises cyclosporin A, Lignosol SFX-65®, and Nutmeg oil.

Another embodiment comprises cyclosporin A, Marasperse 52 CP®, and Nutmeg oil.

Another embodiment comprises cyclosporin A, Marasperse AG®, and Nutmeg oil.

Another embodiment comprises cyclosporin A, Marasperse CBOS-4®, and Nutmeg oil.

Another embodiment comprises cyclosporin A, Ufoxane 2®, and Nutmeg oil.

Another embodiment comprises cyclosporin A, a Methyl Ester, and Nutmeg oil.

Another embodiment comprises cyclosporin A, E.B. Cleaner AK®, and Nutmeg oil.

Another embodiment comprises cyclosporin A, Oleocal ME-70®, and Nutmeg oil.

Another embodiment comprises cyclosporin A, Trade Name Company®, and Nutmeg oil.

Another embodiment comprises cyclosporin A, Methyl Esters (cont'd)®, and Nutmeg oil.

Another embodiment comprises cyclosporin A, Oleocal ME-92®, and Nutmeg oil.

Another embodiment comprises cyclosporin A, Oleocal ME-112®, and Nutmeg oil.

Another embodiment comprises cyclosporin A, Oleocal ME-130®, and Nutmeg oil.

Another embodiment comprises cyclosporin A, a Monbglyceride or a Derivative thereof, and Nutmeg oil.

Another embodiment comprises cyclosporin A, Dynacet 211®, and Nutmeg oil.

Another embodiment comprises cyclosporin A, Hetsorb S-20®, and Nutmeg oil.

Another embodiment comprises cyclosporin A, Imwitor 191®, and Nutmeg oil.

Another embodiment comprises cyclosporin A, Imwitor 370®, and Nutmeg oil.

Another embodiment comprises cyclosporin A, Imwitor 375®, and Nutmeg oil.

Another embodiment comprises cyclosporin A, Imwitor 900®, and Nutmeg oil.

Another embodiment comprises cyclosporin A, Imwitor 945®, and Nutmeg oil.

Another embodiment comprises cyclosporin A, Imwitor 2020®, and Nutmeg oil.

Another embodiment comprises cyclosporin A, Kemester 5500®, and Nutmeg oil.

Another embodiment comprises cyclosporin A, Kemester 6000®, and Nutmeg oil.

Another embodiment comprises cyclosporin A, Magrabar GMC®, and Nutmeg oil.

Another embodiment comprises cyclosporin A, Magrabar GMO-CK®, and Nutmeg oil.

Another embodiment comprises cyclosporin A, Magrabar GPC-10®, and Nutmeg oil.

Another embodiment comprises cyclosporin A, Magrabar MDG-5050®, and Nutmeg oil.

Another embodiment comprises cyclosporin A, Monalube 335®, and Nutmeg oil.

Another embodiment comprises cyclosporin A, Monoglycerides Glyceryl Monestearate Archer Daniels Midland Company®, and Nutmeg oil.

Another embodiment comprises cyclosporin A, Rita GMS®, and Nutmeg oil.

Another embodiment comprises cyclosporin A, Ritamulse SCG®, and Nutmeg oil.

Another embodiment comprises cyclosporin A, Softigen 701®, and Nutmeg oil.

Another embodiment comprises cyclosporin A, Tally 100 Plus®, and Nutmeg oil.

Another embodiment comprises cyclosporin A, a Polyethylene Glycol, and Nutmeg oil.

Another embodiment comprises cyclosporin A, Emulgade PL 68/50®, and Nutmeg oil.

Another embodiment comprises cyclosporin A, Lumulse PEG®, and Nutmeg oil.

Another embodiment comprises cyclosporin A, Rhodasurf PEG-400®, and Nutmeg oil.

Another embodiment comprises cyclosporin A, Rhodasurf PEG-600®, and Nutmeg oil.

Another embodiment comprises cyclosporin A, Witconol PEG-400®, and Nutmeg oil.

Another embodiment comprises cyclosporin A, a Polymeric Surfactant, and Nutrmeg oil.

Another embodiment comprises cyclosporin A, Acritamer PNC-EG®, and Nutmeg oil.

Another embodiment comprises cyclosporin A, Ag-Rho DEP-775®, and Nutmeg oil.

Another embodiment comprises cyclosporin A, APG 325N Glycoside®, and Nutmeg oil.

Another embodiment comprises cyclosporin A, Aristoflex AVC®, and Nutmeg oil.

Another embodiment comprises cyclosporin A, Aristoflex HMB®, and Nutmeg oil.

Another embodiment comprises cyclosporin A, Burco NPS-225®, and Nutmeg oil.

Another embodiment comprises cyclosporin A, Burco NPS-816®, and Nutmeg oil.

Another embodiment comprises cyclosporin A, Chemccinate 5603®, and Nutmeg oil.

Another embodiment comprises cyclosporin A, Cosmedia Guar C-261N®, and Nutmeg oil.

Another embodiment comprises cyclosporin A, Gantrez S-95®, and Nutmeg oil.

Another embodiment comprises cyclosporin A, Glucopon 220 UP®, and Nutmeg oil.

Another embodiment comprises cyclosporin A, Glucopon 225 DK®, and Nutmeg oil.

Another embodiment comprises cyclosporin A, Glucopon 425 N®, and Nutmeg oil.

Another embodiment comprises cyclosporin A, Glucopon 600 UP®, and Nutmeg oil.

Another embodiment comprises cyclosporin A, Glucopon 625 UP®, and Nutmeg oil.

Another embodiment comprises cyclosporin A, Pemulen 1621®, and Nutmeg oil.

Another embodiment comprises cyclosporin A, Pemulen 1622®, and Nutmeg oil.

Another embodiment comprises cyclosporin A, Pemulen TR-1®, and Nutmeg oil.

Another embodiment comprises cyclosporin A, Pemulen TR-2®, and Nutmeg oil.

Another embodiment comprises cyclosporin A, Plantacare 818®, and Nutmeg oil.

Another embodiment comprises cyclosporin A, Plantapon LGC Sorb®, and Nutmeg oil.

Another embodiment comprises cyclosporin A, Plantaren 1200N®, and Nutmeg oil.

Another embodiment comprises cyclosporin A, Plantaren 2000N®, and Nutmeg oil.

Another embodiment comprises cyclosporin A, Viscolam AT 64®, and Nutmeg oil.

Another embodiment comprises cyclosporin A, Viscolam AT 64P®, and Nutmeg oil.

Another embodiment comprises cyclosporin A, Viscolam AT 100®, and Nutmeg oil.

Another embodiment comprises cyclosporin A, Viscolam MAC 7®, and Nutmeg oil.

Another embodiment comprises cyclosporin A, Viscolam SMC 20®, and Nutmeg oil.

Another embodiment comprises cyclosporin A, Witbreak RTC-323®, and Nutmeg oil.

Another embodiment comprises cyclosporin A, WSI 3700®, and Nutmeg oil.

Another embodiment comprises cyclosporin A, a Propoxylated or Ethoxylated Fatty Acid, Alcohol, or Alkyl Phenol, and Nutmeg oil.

Another embodiment comprises cyclosporin A, Antarox AA-60®, and Nutmeg oil.

Another embodiment comprises cyclosporin A, Antarox LF-224®, and Nutmeg oil.

Another embodiment comprises cyclosporin A, Burcomul DFE-45®, and Nutmeg oil.

Another embodiment comprises cyclosporin A, Burcoterge LFE-1000®, and Nutmeg oil.

Another embodiment comprises cyclosporin A, Chemal LF-25B®, and Nutmeg oil.

Another embodiment comprises cyclosporin A, Chemal LF-40B®, and Nutmeg oil.

Another embodiment comprises cyclosporin A, Dehypon LS-36®, and Nutmeg oil.

Another embodiment comprises cyclosporin A, Dehypon LS-36®, and Nutmeg oil.

Another embodiment comprises cyclosporin A, Dehypon LS-54®, and Nutmeg oil.

Another embodiment comprises cyclosporin A, Dehypon LS-54®, and Nutmeg oil.

Another embodiment comprises cyclosporin A, Delonic 100 VLF®, and Nutmeg oil.

Another embodiment comprises cyclosporin A, Delonic LF-60 MOD®, and Nutmeg oil.

Another embodiment comprises cyclosporin A, Empiderm B®, and Nutmeg oil.

Another embodiment comprises cyclosporin A, Ethylan 1206®, and Nutmeg oil.

Another embodiment comprises cyclosporin A, Ethylan NS-500K®, and Nutmeg oil.

Another embodiment comprises cyclosporin A, Ethylan NS-500LQ®, and Nutmeg oil.

Another embodiment comprises cyclosporin A, Genapol 1392®, and Nutmeg oil.

Another embodiment comprises cyclosporin A, Genapol 2317®, and Nutmeg oil.

Another embodiment comprises cyclosporin A, Genapol 26EP710®, and Nutmeg oil.

Another embodiment comprises cyclosporin A, Genapol EP 1022®, and Nutmeg oil.

Another embodiment comprises cyclosporin A, Genapol EP 1024®, and Nutmeg oil.

Another embodiment comprises cyclosporin A, Genapol EP 6068®, and Nutmeg oil.

Another embodiment comprises cyclosporin A, Genapol NP915®, and Nutmeg oil.

Another embodiment comprises cyclosporin A, Kieralon MFB®, and Nutmeg oil.

Another embodiment comprises cyclosporin A, Lumisolve CSA-80 V®, and Nutmeg oil.

Another embodiment comprises cyclosporin A, Marlowet 5001®, and Nutmeg oil.

Another embodiment comprises cyclosporin A, Marlox FK 64®, and Nutmeg oil.

Another embodiment comprises cyclosporin A, Marlox MO 124®, and Nutmeg oil.

Another embodiment comprises cyclosporin A, Marlox S 58®, and Nutmeg oil.

Another embodiment comprises cyclosporin A, Nonatell 1003®, and Nutmeg oil.

Another embodiment comprises cyclosporin A, Nonatell 1038®, and Nutmeg oil.

Another embodiment comprises cyclosporin A, Nonatell 1052®, and Nutmeg oil.

Another embodiment comprises cyclosporin A, Nonatell 1061®, and Nutmeg oil.

Another embodiment comprises cyclosporin A, Nonatell 1075®, and Nutmeg oil.

Another embodiment comprises cyclosporin A, Nonatell 1088®, and Nutmeg oil.

Another embodiment comprises cyclosporin A, Nonatell 1123®, and Nutmeg oil.

Another embodiment comprises cyclosporin A, Nonatell 1153®, and Nutmeg oil.

Another embodiment comprises cyclosporin A, Nonatell 1161®, and Nutmeg oil.

Another embodiment comprises cyclosporin A, Nonatell 1172®, and Nutmeg oil.

Another embodiment comprises cyclosporin A, Nonatell 1181®, and Nutmeg oil.

Another embodiment comprises cyclosporin A, Norfox 36®, and Nutmeg oil.

Another embodiment comprises cyclosporin A, Procetyl AWS®, and Nutmeg oil.

Another embodiment comprises cyclosporin A, Sandoxylate SX 412®, and Nutmeg oil.

Another embodiment comprises cyclosporin A, Sandoxylate SX 418®, and Nutmeg oil.

Another embodiment comprises cyclosporin A, Surfonic JL-80X®, and Nutmeg oil.

Another embodiment comprises cyclosporin A, Surfonic JL-80X-B1®, and Nutmeg oil.

Another embodiment comprises cyclosporin A, Surfonic L4-29X®, and Nutmeg oil.

Another embodiment comprises cyclosporin A, Surfonic LF®, and Nutmeg oil.

Another embodiment comprises cyclosporin A, T-Det A826®, and Nutmeg oil.

Another embodiment comprises cyclosporin A, T-Det LF-416®, and Nutmeg oil.

Another embodiment comprises cyclosporin A, Tergitol Min-Foam 1X®, and Nutmeg oil.

Another embodiment comprises cyclosporin A, Tergitol Min-Foam 2X®, and Nutmeg oil.

Another embodiment comprises cyclosporin A, Triton CF-21®, and Nutmeg oil.

Another embodiment comprises cyclosporin A, Triton CF-76®, and Nutmeg oil.

Another embodiment comprises cyclosporin A, Triton XL-80N®, and Nutmeg oil.

Another embodiment comprises cyclosporin A, Witconol NS-98®, and Nutmeg oil.

Another embodiment comprises cyclosporin A, Witconol NS-108LQ®, and Nutmeg oil.

Another embodiment comprises cyclosporin A, Witconol NS-145®, and Nutmeg oil.

Another embodiment comprises cyclosporin A, Witconol NS-179®, and Nutmeg oil.

Another embodiment comprises cyclosporin A, a Protein-based Surfactant, and Nutmeg oil.

Another embodiment comprises cyclosporin A, AminoFoam W®, and Nutmeg oil.

Another embodiment comprises cyclosporin A, Amiter LGOD-2®, and Nutmeg oil.

Another embodiment comprises cyclosporin A, Amiter LGS-2®, and Nutmeg oil.

Another embodiment comprises cyclosporin A, Amiter LGS-5®, and Nutmeg oil.

Another embodiment comprises cyclosporin A, Lamepon S®, and Nutmeg oil.

Another embodiment comprises cyclosporin A, Lamepon S®, and Nutmeg oil.

Another embodiment comprises cyclosporin A, Maypon 4C®, and Nutmeg oil.

Another embodiment comprises cyclosporin A, May-Tein C®, and Nutmeg oil.

Another embodiment comprises cyclosporin A, May-Tein CT®, and Nutmeg oil.

Another embodiment comprises cyclosporin A, May-Tein KTS®, and Nutmeg oil.

Another embodiment comprises cyclosporin A, May-Tein SY®, and Nutmeg oil.

Another embodiment comprises cyclosporin A, Plantapon S®, and Nutmeg oil.

Another embodiment comprises cyclosporin A, Proteol APL®, and Nutmeg oil.

Another embodiment comprises cyclosporin A, Proteol OAT®, and Nutmeg oil.

Another embodiment comprises cyclosporin A, Pyroter CPI-40®, and Nutmeg oil.

Another embodiment comprises cyclosporin A, Pyroter GPI-25®, and Nutmeg oil.

Another embodiment comprises cyclosporin A, Supro-Tein S®, and Nutmeg oil.

Another embodiment comprises cyclosporin A, Supro-Tein V®, and Nutmeg oil.

Another embodiment comprises cyclosporin A,® and Nutmeg oil.

Another embodiment comprises cyclosporin A, a Sarcosine Derivative®, and Nutmeg oil.

Another embodiment comprises cyclosporin A, Crodasinic LS-30®, and Nutmeg oil.

Another embodiment comprises cyclosporin A, Vanseal CS®, and Nutmeg oil.

Another embodiment comprises cyclosporin A, Vanseal LS®, and Nutmeg oil.

Another embodiment comprises cyclosporin A, Vanseal MS®, and Nutmeg oil.

Another embodiment comprises cyclosporin A, Vanseal NACS-30®, and Nutmeg oil.

Another embodiment comprises cyclosporin A, Vanseal NALS-95®, and Nutmeg oil.

Another embodiment comprises cyclosporin A, Vanseal OS®, and Nutmeg oil.

Another embodiment comprises cyclosporin A,® and Nutmeg oil.

Another embodiment comprises cyclosporin A, a Silicone-based Surfactant®, and Nutmeg oil.

Another embodiment comprises cyclosporin A, Abil-B-9950®, and Nutmeg oil.

Another embodiment comprises cyclosporin A, Abil Care 85®, and Nutmeg oil.

Another embodiment comprises cyclosporin A, Abil EM 90®, and Nutmeg oil.

Another embodiment comprises cyclosporin A, Abil EM 97®, and Nutmeg oil.

Another embodiment comprises cyclosporin A, Abil WE-09®, and Nutmeg oil.

Another embodiment comprises cyclosporin A, Dow Corning 1248 Fluid®, and Nutmeg oil.

Another embodiment comprises cyclosporin A, Dow Corning 3225C®, and Nutmeg oil.

Another embodiment comprises cyclosporin A, Dow Corning 5200®, and Nutmeg oil.

Another embodiment comprises cyclosporin A, Dow Corning Q4-3667®, and Nutmeg oil.

Another embodiment comprises cyclosporin A, Monasil PCA®, and Nutmeg oil.

Another embodiment comprises cyclosporin A, Monasil PDM®, and Nutmeg oil.

Another embodiment comprises cyclosporin A, Monasil PLN®, and Nutmeg oil.

Another embodiment comprises cyclosporin A, Polyderm PPI-SI-WS®, and Nutmeg oil.

Another embodiment comprises cyclosporin A, Troysol 380W®, and Nutmeg oil.

Another embodiment comprises cyclosporin A, Troysol S366®, and Nutmeg oil.

Another embodiment comprises cyclosporin A, a Sorbitan Derivative, and Nutmeg oil.

Another embodiment comprises cyclosporin A, Alkamuls SML®, and Nutmeg oil.

Another embodiment comprises cyclosporin A, Alkamuls SMO®, and Nutmeg oil.

Another embodiment comprises cyclosporin A, Alkamuls STO®, and Nutmeg oil.

Another embodiment comprises cyclosporin A, Arlacel 20®, and Nutmeg oil.

Another embodiment comprises cyclosporin A, Arlacel 40®, and Nutmeg oil.

Another embodiment comprises cyclosporin A, Arlacel 60®, and Nutmeg oil.

Another embodiment comprises cyclosporin A, Arlacel 80®, and Nutmeg oil.

Another embodiment comprises cyclosporin A, Arlacel C®, and Nutmeg oil.

Another embodiment comprises cyclosporin A, Armul 21®, and Nutmeg oil.

Another embodiment comprises cyclosporin A, Atlox 80®, and Nutmeg oil.

Another embodiment comprises cyclosporin A, Atlox 847®, and Nutmeg oil.

Another embodiment comprises cyclosporin A, Atlox 1045A®, and Nutmeg oil.

Another embodiment comprises cyclosporin A, Canarcel 20®, and Nutmeg oil.

Another embodiment comprises cyclosporin A, Canarcel 60®, and Nutmeg oil.

Another embodiment comprises cyclosporin A, Canarcel 80®, and Nutmeg oil.

Another embodiment comprises cyclosporin A, Canarcel TW 20®, and Nutmeg oil.

Another embodiment comprises cyclosporin A, Canarcel TW 60®, and Nutmeg oil.

Another embodiment comprises cyclosporin A, Canarcel TW 80®, and Nutmeg oil.

Another embodiment comprises cyclosporin A, Coladet BSB-P®, and Nutmeg oil.

Another embodiment comprises cyclosporin A, Customulse O-20®, and Nutmeg oil.

Another embodiment comprises cyclosporin A, Dehymuls E®, and Nutmeg oil.

Another embodiment comprises cyclosporin A, DeSotan SMO®, and Nutmeg oil.

Another embodiment comprises cyclosporin A, DeSotan SMO-20®, and Nutmeg oil.

Another embodiment comprises cyclosporin A, DeSotan SMT®, and Nutmeg oil.

Another embodiment comprises cyclosporin A, DeSotan SMT-20®, and Nutmeg oil.

Another embodiment comprises cyclosporin A, Durfax 60®, and Nutmeg oil.

Another embodiment comprises cyclosporin A, Durfax 65®, and Nutmeg oil.

Another embodiment comprises cyclosporin A, Durfax 80®, and Nutmeg oil.

Another embodiment comprises cyclosporin A, Durtan 60®, and Nutmeg oil.

Another embodiment comprises cyclosporin A, Durtan 65®, and Nutmeg oil.

Another embodiment comprises cyclosporin A, Liposorb L®, and Nutmeg oil.

Another embodiment comprises cyclosporin A, Liposorb L-10®, and Nutmeg oil.

Another embodiment comprises cyclosporin A, Liposorb L-20®, and Nutmeg oil.

Another embodiment comprises cyclosporin A, Liposorb O®, and Nutmeg oil.

Another embodiment comprises cyclosporin A, Liposorb O-20®, and Nutmeg oil.

Another embodiment comprises cyclosporin A, Liposorb P®, and Nutmeg oil.

Another embodiment comprises cyclosporin A, Liposorb P-20®, and Nutmeg oil.

Another embodiment comprises cyclosporin A, Liposorb S®, and Nutmeg oil.

Another embodiment comprises cyclosporin A, Liposorb S-20®, and Nutmeg oil.

Another embodiment comprises cyclosporin A, Liposorb SQO®, and Nutmeg oil.

Another embodiment comprises cyclosporin A, Liposorb TO®, and Nutmeg oil.

Another embodiment comprises cyclosporin A, Liposorb TS®, and Nutmeg oil.

Another embodiment comprises cyclosporin A, Liposorb TS-20®, and Nutmeg oil.

Another embodiment comprises cyclosporin A, Lumisorb PS®, and Nutmeg oil.

Another embodiment comprises cyclosporin A, Lumisorb SMO (T)®, and Nutmeg oil.

Another embodiment comprises cyclosporin A, Lumisorb SMS K®, and Nutmeg oil.

Another embodiment comprises cyclosporin A, Lumisorb SSO®, and Nutmeg oil.

Another embodiment comprises cyclosporin A, Lumisorb STS K®, and Nutmeg oil.

Another embodiment comprises cyclosporin A, Lumisorb STT®, and Nutmeg oil.

Another embodiment comprises cyclosporin A, Magrabar SMO®, and Nutmeg oil.

Another embodiment comprises cyclosporin A, Magrabar SMO-VEG®, and Nutmeg oil.

Another embodiment comprises cyclosporin A, Magrabar SMT®, and Nutmeg oil.

Another embodiment comprises cyclosporin A, Magrabar STO®, and Nutmeg oil.

Another embodiment comprises cyclosporin A, Miracare BC-27®, and Nutmeg oil.

Another embodiment comprises cyclosporin A, Ritabate 20®, and Nutmeg oil.

Another embodiment comprises cyclosporin A, Ritabate 40®, and Nutmeg oil.

Another embodiment comprises cyclosporin A, Ritabate 60®, and Nutmeg oil.

Another embodiment comprises cyclosporin A, Ritabate 80®, and Nutmeg oil.

Another embodiment comprises cyclosporin A, T-Maz®, and Nutmeg oil.

Another embodiment comprises cyclosporin A, Tego SML®, and Nutmeg oil.

Another embodiment comprises cyclosporin A, Tego SML 20®, and Nutmeg oil.

Another embodiment comprises cyclosporin A, Tego SMO 80 V®, and Nutmeg oil.

Another embodiment comprises cyclosporin A, Tego SMO V®, and Nutmeg oil.

Another embodiment comprises cyclosporin A, Tego SMS®, and Nutmeg oil.

Another embodiment comprises cyclosporin A, Tego STO V®, and Nutmeg oil.

Another embodiment comprises cyclosporin A, Tween 21®, and Nutmeg oil.

Another embodiment comprises cyclosporin A, Tween 40®, and Nutmeg oil.

Another embodiment comprises cyclosporin A, Tween 60®, and Nutmeg oil.

Another embodiment comprises cyclosporin A, Tween 60 K®, and Nutmeg oil.

Another embodiment comprises cyclosporin A, Tween 61®, and Nutmeg oil.

Another embodiment comprises cyclosporin A, Tween 65®, and Nutmeg oil.

Another embodiment comprises cyclosporin A, Tween 80®, and Nutmeg oil.

Another embodiment comprises cyclosporin A, Tween 80 K®, and Nutmeg oil.

Another embodiment comprises cyclosporin A, Tween 81®, and Nutmeg oil.

Another embodiment comprises cyclosporin A, Tween 85®, and Nutmeg oil.

Another embodiment comprises cyclosporin A, a Sucrose or Glucose Ester, or Derivative thereof, and Nutmeg oil.

Another embodiment comprises cyclosporin A, DeSulf GOS-P-60WCG®, and Nutmeg oil.

Another embodiment comprises cyclosporin A, Glucam E-20 Distearate®, and Nutmeg oil.

Another embodiment comprises cyclosporin A, Glucamate DOE-120®, and Nutmeg oil.

Another embodiment comprises cyclosporin A, Glucamate SSE-20®, and Nutmeg oil.

Another embodiment comprises cyclosporin A, Glucate DO®, and Nutmeg oil.

Another embodiment comprises cyclosporin A, Glucate SS®, and Nutmeg oil.

Another embodiment comprises cyclosporin A, Glucopon 425 UP®, and Nutmeg oil.

Another embodiment comprises cyclosporin A, Isolan IS®, and Nutmeg oil.

Another embodiment comprises cyclosporin A, Mazon 40®, and Nutmeg oil.

Another embodiment comprises cyclosporin A, Montanov 82®, and Nutmeg oil.

Another embodiment comprises cyclosporin A, Montanov 202®, and Nutmeg oil.

Another embodiment comprises cyclosporin A, Montanov S®, and Nutmeg oil.

Another embodiment comprises cyclosporin A, Rheozan®, and Nutmeg oil.

Another embodiment comprises cyclosporin A, Simulsol AS 48®, and Nutmeg oil.

Another embodiment comprises cyclosporin A, Simulsol SL 4®, and Nutmeg oil.

Another embodiment comprises cyclosporin A, Simulsol SL 10®, and Nutmeg oil.

Another embodiment comprises cyclosporin A, Simulsol SL 11 W®, and Nutmeg oil.

Another embodiment comprises cyclosporin A, Simulsol SL 55®, and Nutmeg oil.

Another embodiment comprises cyclosporin A, Suga Nate 100 and 160®, and Nutmeg oil.

Another embodiment comprises cyclosporin A, Tego Care 450®, and Nutmeg oil.

Another embodiment comprises cyclosporin A, Tego Care CG 90®, and Nutmeg oil.

Another embodiment comprises cyclosporin A, Tego Care PS®, and Nutmeg oil.

Another embodiment comprises cyclosporin A, Tegosoft PSE 141 G®, and Nutmeg oil.

Another embodiment comprises cyclosporin A, Tegotens G 826®, and Nutmeg oil.

Another embodiment comprises cyclosporin A, Triton BG-10 (70%)®, and Nutmeg oil.

Another embodiment comprises cyclosporin A, Triton CG-110 (60%)®, and Nutmeg oil.

Another embodiment comprises cyclosporin A, Wickenol 545®, and Nutmeg oil.

Another embodiment comprises cyclosporin A, an alcohol, and Tea tree oil.

Another embodiment comprises cyclosporin A, Diglycerof®, and Tea tree oil.

Another embodiment comprises cyclosporin A, Hetoxide GT-80®, and Tea tree oil.

Another embodiment comprises cyclosporin A, Lexemul BEO®, and Tea tree oil.

Another embodiment comprises cyclosporin A, Polyglycerol-3®, and Tea tree oil.

Another embodiment comprises cyclosporin A, Redicote E Series®, and Tea tree oil.

Another embodiment comprises cyclosporin A, Simulsol OX 1005L®, and Tea tree oil.

Another embodiment comprises cyclosporin A, Stanfax 567®, and Tea tree oil.

Another embodiment comprises cyclosporin A, TA-1618®, and Tea tree oil.

Another embodiment comprises cyclosporin A, Witconol H-31A®, and Tea tree oil.

Another embodiment comprises cyclosporin A, an amine oxide, and Tea tree oil.

Another embodiment comprises cyclosporin A, AO-405®, and Tea tree oil.

Another embodiment comprises cyclosporin A, AO-455®, and Tea tree oil.

Another embodiment comprises cyclosporin A, AO 728 Special®, and Tea tree oil.

Another embodiment comprises cyclosporin A, Barlox 12®, and Tea tree oil.

Another embodiment comprises cyclosporin A, Barlox 14®, and Tea tree oil.

Another embodiment comprises cyclosporin A, Burcoxide Lo®, and Tea tree oil.

Another embodiment comprises cyclosporin A, Caloxamine LO®, and Tea tree oil.

Another embodiment comprises cyclosporin A, Chemoxide CAW®, and Tea tree oil.

Another embodiment comprises cyclosporin A, Chemoxide LM-30®, and Tea tree oil.

Another embodiment comprises cyclosporin A, Chemoxide LO®, and Tea tree oil.

Another embodiment comprises cyclosporin A, Chemoxide MO®, and Tea tree oil.

Another embodiment comprises cyclosporin A, Colalux CAO-35®, and Tea tree oil.

Another embodiment comprises cyclosporin A, Colalux LO®, and Tea tree oil.

Another embodiment comprises cyclosporin A, DeMox CAPO®, and Tea tree oil.

Another embodiment comprises cyclosporin A, DeMox CSG-30®, and Tea tree oil.

Another embodiment comprises cyclosporin A, DeMox LAO®, and Tea tree oil.

Another embodiment comprises cyclosporin A, Emcol L®, and Tea tree oil.

Another embodiment comprises cyclosporin A, Empigen OB®, and Tea tree oil.

Another embodiment comprises cyclosporin A, Empigen OS/A®, and Tea tree oil.

Another embodiment comprises cyclosporin A, Foamox CDO®, and Tea tree oil.

Another embodiment comprises cyclosporin A, Foamox DMM®, and Tea tree oil.

Another embodiment comprises cyclosporin A, Foamox DMS®, and Tea tree oil.

Another embodiment comprises cyclosporin A, Genaminox KC®, and Tea tree oil.

Another embodiment comprises cyclosporin A, Genaminox LA®, and Tea tree oil.

Another embodiment comprises cyclosporin A, Hartofoam SAO®, and Tea tree oil.

Another embodiment comprises cyclosporin A, Hartox DMCD®, and Tea tree oil.

Another embodiment comprises cyclosporin A, Lipowax DAT®, and Tea tree oil.

Another embodiment comprises cyclosporin A, Lipowax PB Pastilles®, and Tea tree oil.

Another embodiment comprises cyclosporin A, Mackamine C8®, and Tea tree oil.

Another embodiment comprises cyclosporin A, Mackamine C10®, and Tea tree oil.

Another embodiment comprises cyclosporin A, Mackamine C14®, and Tea tree oil.

Another embodiment comprises cyclosporin A, Mackamine CAO®, and Tea tree oil.

Another embodiment comprises cyclosporin A, Mackamine CO®, and Tea tree oil.

Another embodiment comprises cyclosporin A, Mackamine LO®, and Tea tree oil.

Another embodiment comprises cyclosporin A, Mackamine O2®, and Tea tree oil.

Another embodiment comprises cyclosporin A, Mackamine SAO®, and Tea tree oil.

Another embodiment comprises cyclosporin A, Mackamine SO®, and tea tree oil.

Another embodiment comprises cyclosporin A, Mazox KCAO®, and Tea tree oil.

Another embodiment comprises cyclosporin A, Monalac MO®, and Tea tree oil.

Another embodiment comprises cyclosporin A, Norfox LDA®, and Tea tree oil.

Another embodiment comprises cyclosporin A, Rhodamox LO®, and Tea tree oil.

Another embodiment comprises cyclosporin A, Schercamox C-AA®, and Tea tree oil.

Another embodiment comprises cyclosporin A, Schercamox DMA®, and Tea tree oil.

Another embodiment comprises cyclosporin A, Schercamox DML®, and Tea tree oil.

Another embodiment comprises cyclosporin A, Schercamox DMM®, and Tea tree oil.

Another embodiment comprises cyclosporin A, Schercamox DMS®, and Tea tree oil.

Another embodiment comprises cyclosporin A, Tegotens DO®, and Tea tree oil.

Another embodiment comprises cyclosporin A, Tomah AO-14-2®, and Tea tree oil.

Another embodiment comprises cyclosporin A, Triaminox CDO®, and Tea tree oil.

Another embodiment comprises cyclosporin A, a block polymer, and Tea tree oil.

Another embodiment comprises cyclosporin A, AL 2070®, and Tea tree oil.

Another embodiment comprises cyclosporin A, Antarox 17-R-2®, and Tea tree oil.

Another embodiment comprises cyclosporin A, Antarox 25-R-2®, and Tea tree oil.

Another embodiment comprises cyclosporin A, Antarox 31-R-1®, and Tea tree oil.

Another embodiment comprises cyclosporin A, Antarox P-84®, and Tea tree oil.

Another embodiment comprises cyclosporin A, Antarox P-104/H®, and Tea tree oil.

Another embodiment comprises cyclosporin A, Arnox BP-Series®, and Tea tree oil.

Another embodiment comprises cyclosporin A, Chemonic 435®, and Tea tree oil.

Another embodiment comprises cyclosporin A, Chemonic D-25®, and Tea tree oil.

Another embodiment comprises cyclosporin A, Chemonic PL Series®, and Tea tree oil.

Another embodiment comprises cyclosporin A, Ethox L-121®, and Tea tree oil.

Another embodiment comprises cyclosporin A, Ethox L-122®, and Tea tree oil.

Another embodiment comprises cyclosporin A, Genapol PF-10®, and Tea tree oil.

Another embodiment comprises cyclosporin A, Genapol PF-20®, and Tea tree oil.

Another embodiment comprises cyclosporin A, Genapol PF-40A®, and Tea tree oil.

Another embodiment comprises cyclosporin A, Nonox 2-LF®, and Tea tree oil.

Another embodiment comprises cyclosporin A, Simulsol NW 342®, and Tea tree oil.

Another embodiment comprises cyclosporin A, T-Det BP-1®, and Tea tree oil.

Another embodiment comprises cyclosporin A, T-Det XD®, and Tea tree oil.

Another embodiment comprises cyclosporin A, T-Det XH®, and Tea tree oil.

Another embodiment comprises cyclosporin A, Triton CF-32®, and Tea tree oil.

Another embodiment comprises cyclosporin A, Witconol 171®, and Tea tree oil.

Another embodiment comprises cyclosporin A, Witconol 324®, and Tea tree oil.

Another embodiment comprises cyclospQrin A, Witconol 324D®, and Tea tree oil.

Another embodiment comprises cyclosporin A, Witconol PD-2000®, and Tea tree oil.

Another embodiment comprises cyclosporin A, a Carboxylated Alcohol or Alkylphenol Ethoxylate, and Tea tree oil.

Another embodiment comprises cyclosporin A, Emcol CN-6®, and Tea tree oil.

Another embodiment comprises cyclosporin A, Ethcarb®, and Tea tree oil.

Another embodiment comprises cyclosporin A, Gemtex WNT-Conc®, and Tea tree oil.

Another embodiment comprises cyclosporin A, Incrodet TD7-C®, and Tea tree oil.

Another embodiment comprises cyclosporin A, Marlinat CM 105/80®, and Tea tree oil.

Another embodiment comprises cyclosporin A, Marlowet 1072®, and Tea tree oil.

Another embodiment comprises cyclosporin A, Marlowet 4530®, and Tea tree oil.

Another embodiment comprises cyclosporin A, Marlowet 4530 LF®, and Tea tree oil.

Another embodiment comprises cyclosporin A, Marlowet 4534®, and Tea tree oil.

Another embodiment comprises cyclosporin A, Marlowet 4538®, and Tea tree oil.

Another embodiment comprises cyclosporin A, Marlowet 4539®, and Tea tree oil.

Another embodiment comprises cyclosporin A, Marlowet 4539 LF®, and Tea tree oil.

Another embodiment comprises cyclosporin A, Marlowet 4541®, and Tea tree oil.

Another embodiment comprises cyclosporin A, Miranate LEC-80®, and Tea tree oil.

Another embodiment comprises cyclosporin A, Sandopan B®, and Tea tree oil.

Another embodiment comprises cyclosporin A, Sandopan B Modified®, and Tea tree oil.

Another embodiment comprises cyclosporin A, Sandopan LS-24 Gel®, and Tea tree oil.

Another embodiment comprises cyclosporin A, Surfine T-A®, and Tea tree oil.

Another embodiment comprises cyclosporin A, a Carboxylic Acid/Fatty Acid, and Tea tree oil.

Another embodiment comprises cyclosporin A, Colaterge RAM®, and Tea tree oil.

Another embodiment comprises cyclosporin A, Colatrope INC®, and Tea tree oil.

Another embodiment comprises cyclosporin A, Crodacid B®, and Tea tree oil.

Another embodiment comprises cyclosporin A, DeTrope CA-100®, and Tea tree oil.

Another embodiment comprises cyclosporin A, Latol MTO®, and Tea tree oil.

Another embodiment comprises cyclosporin A, Lumulse CC-33 K®, and Tea tree oil.

Another embodiment comprises cyclosporin A, Mulls 2218®, and Tea tree oil.

Another embodiment comprises cyclosporin A, OL-600®, and Tea tree oil.

Another embodiment comprises cyclosporin A, OL-800®, and Tea tree oil.

Another embodiment comprises cyclosporin A, R-910®, and Tea tree oil.

Another embodiment comprises cyclosporin A, S-210®, and Tea tree oil.

Another embodiment comprises cyclosporin A, Sandopan DTC Acid®, and Tea tree oil.

Another embodiment comprises cyclosporin A, Sandopan LS 24N®, and Tea tree oil.

Another embodiment comprises cyclosporin A, Sandopan MA-18®, and Tea tree oil.

Another embodiment comprises cyclosporin A, an Ethoxylated Alcohol, and Tea tree oil.

Another embodiment comprises cyclosporin A, Adsee 799®, and Tea tree oil.

Another embodiment comprises cyclosporin A, Adsee 799®, and Tea tree oil.

Another embodiment comprises cyclosporin A, Alfonic 610-3.5®, and Tea tree oil.

Another embodiment comprises cyclosporin A, Alfonic 810-2®, and Tea tree oil.

Another embodiment comprises cyclosporin A, Alfonic 810-6®, and Tea tree oil.

Another embodiment comprises cyclosporin A, Alfonic 1012-3®, and Tea tree oil.

Another embodiment comprises cyclosporin A, Alfonic 1012-5®, and Tea tree oil.

Another embodiment comprises cyclosporin A, Alfonic 1216CO-1.5®, and Tea tree oil.

Another embodiment comprises cyclosporin A, Alfonic 1216CO-7®, and Tea tree oil.

Another embodiment comprises cyclosporin A, Alfonic 1412-3®, and Tea tree oil.

Another embodiment comprises cyclosporin A, Alfonic 1412-7®, and Tea tree oil.

Another embodiment comprises cyclosporin A, Arlasolve 200®, and Tea tree oil.

Another embodiment comprises cyclosporin A, Arlasolve 200 Liquid®, and Tea tree oil.

Another embodiment comprises cyclosporin A, Armix 180-C®, and Tea tree oil.

Another embodiment comprises cyclosporin A, Armix 183®, and Tea tree oil.

Another embodiment comprises cyclosporin A, Armul 2404®, and Tea tree oil.

Another embodiment comprises cyclosporin A, Armul 2404®, and Tea tree oil.

Another embodiment comprises cyclosporin A, Atlas EMJ-C®, and Tea tree oil.

Another embodiment comprises cyclosporin A, Atlas G-2109®, and Tea tree oil.

Another embodiment comprises cyclosporin A, Atlas G-3886®, and Tea tree oil.

Another embodiment comprises cyclosporin A, Atlas G-3890®, and Tea tree oil.

Another embodiment comprises cyclosporin A, Bio Soft E-200®, and Tea tree oil.

Another embodiment comprises cyclosporin A, Bio Soft E-300®, and Tea tree oil.

Another embodiment comprises cyclosporin A, Bio Soft E-400®, and Tea tree oil.

Another embodiment comprises cyclosporin A, Bio Soft EN 600®, and Tea tree oil.

Another embodiment comprises cyclosporin A, Bio Soft TD-400®, and Tea tree oil.

Another embodiment comprises cyclosporin A, Bio Soft TD-630®, and Tea tree oil.

Another embodiment comprises cyclosporin A, Brij 30®, and Tea tree oil.

Another embodiment comprises cyclosporin A, Brij 52®, and Tea tree oil.

Another embodiment comprises cyclosporin A, Brij 56®, and Tea tree oil.

Another embodiment comprises cyclosporin A, Brij 58®, and Tea tree oil.

Another embodiment comprises cyclosporin A, Brij 72®, and Tea tree oil.

Another embodiment comprises cyclosporin A, Brij 76®, and Tea tree oil.

Another embodiment comprises cyclosporin A, Brij 78®, and Tea tree oil.

Another embodiment comprises cyclosporin A, Brij 93®, and Tea tree oil.

Another embodiment comprises cyclosporin A, Brij 97®, and Tea tree oil.

Another embodiment comprises cyclosporin A, Brij 98®, and Tea tree oil.

Another embodiment comprises cyclosporin A, Brij 700®, and Tea tree oil.

Another embodiment comprises cyclosporin A, Brij 700 S®, and Tea tree oil.

Another embodiment comprises cyclosporin A, Brij 721®, and Tea tree oil.

Another embodiment comprises cyclosporin A, Brij 721 S®, and Tea tree oil.

Another embodiment comprises cyclosporin A, Burcoterge CDG®, and Tea tree oil.

Another embodiment comprises cyclosporin A, Canasol AT 600®, and Tea tree oil.

Another embodiment comprises cyclosporin A, Canasol AT 800®, and Tea tree oil.

Another embodiment comprises cyclosporin A, Canasol AT 1200®, and Tea tree oil.

Another embodiment comprises cyclosporin A, Canasol BJ 35®, and Tea tree oil.

Another embodiment comprises cyclosporin A, Canasol BJ 36®, and Tea tree oil.

Another embodiment comprises cyclosporin A, Canasol BJ 52®, and Tea tree oil.

Another embodiment comprises cyclosporin A, Canasol BJ 58®, and Tea tree oil.

Another embodiment comprises cyclosporin A, Canasol BJ 72®, and Tea tree oil.

Another embodiment comprises cyclosporin A, Canasol BJ 78®, and Tea tree oil.

Another embodiment comprises cyclosporin A, Canasol BJ 98®, and Tea tree oil.

Another embodiment comprises cyclosporin A, Canasol BJ 307®, and Tea tree oil.

Another embodiment comprises cyclosporin A, Cerfak 1400®, and Tea tree oil.

Another embodiment comprises cyclosporin A, Cetomacrogol 1000 BP®, and Tea tree oil.

Another embodiment comprises cyclosporin A, Chemonic C-2®, and Tea tree oil.

Another embodiment comprises cyclosporin A, Chemonic C-10®, and Tea tree oil.

Another embodiment comprises cyclosporin A, Chemonic C-20®, and Tea tree oil.

Another embodiment comprises cyclosporin A, Chemonic CT-12®, and Tea tree oil.

Another embodiment comprises cyclosporin A, Chemonic CT-20®, and Tea tree oil.

Another embodiment comprises cyclosporin A, Chemonic CT-30®, and Tea tree oil.

Another embodiment comprises cyclosporin A, Chemonic CT-55®, and Tea tree oil.

Another embodiment comprises cyclosporin A, Chemonic G-7®, and Tea tree oil.

Another embodiment comprises cyclosporin A, Chemonic G-26®, and Tea tree oil.

Another embodiment comprises cyclosporin A, Chemonic L-4®, and Tea tree oil.

Another embodiment comprises cyclosporin A, Chemonic L-7®, and Tea tree oil.

Another embodiment comprises cyclosporin A, Chemonic L-12®, and Tea tree oil.

Another embodiment comprises cyclosporin A, Chemonic L-23®, and Tea tree oil.

Another embodiment comprises cyclosporin A, Chemonic O-2®, and Tea tree oil.

Another embodiment comprises cyclosporin A, Chemonic O-5®, and Tea tree oil.

Another embodiment comprises cyclosporin A, Chemonic O-10®, and Tea tree oil.

Another embodiment comprises cyclosporin A, Chemonic O-20®, and Tea tree oil.

Another embodiment comprises cyclosporin A, Chemonic S-2®, and Tea tree oil.

Another embodiment comprises cyclosporin A, Chemonic S-10®, and Tea tree oil.

Another embodiment comprises cyclosporin A, Chemonic S-20®, and Tea tree oil.

Another embodiment comprises cyclosporin A, Colamulse FE®, and Tea tree oil.

Another embodiment comprises cyclosporin A, Cremophor A 20®, and Tea tree oil.

Another embodiment comprises cyclosporin A, Cremophor SA 2®, and Tea tree oil.

Another embodiment comprises cyclosporin A, Dehydol 100®, and Tea tree oil.

Another embodiment comprises cyclosporin A, Dehydol O-4®, and Tea tree oil.

Another embodiment comprises cyclosporin A, Delonic C-18®, and Tea tree oil.

Another embodiment comprises cyclosporin A, DeSonic 6T®, and Tea tree oil.

Another embodiment comprises cyclosporin A, DeSonic 9D®, and Tea tree oil.

Another embodiment comprises cyclosporin A, DeSonic 9T®, and Tea tree oil.

Another embodiment comprises cyclosporin A, DeSonic 12D®, and Tea tree oil.

Another embodiment comprises cyclosporin A, DeSonic 12T®, and Tea tree oil.

Another embodiment comprises cyclosporin A, DeSonic 15T®, and Tea tree oil.

Another embodiment comprises cyclosporin A, DeSonic TDA-9®, and Tea tree oil.

Another embodiment comprises cyclosporin A, DeThox GLG-7®, and Tea tree oil.

Another embodiment comprises cyclosporin A, DeThox GLG-26®, and Tea tree oil.

Another embodiment comprises cyclosporin A, DeThox LA-4®, and Tea tree oil.

Another embodiment comprises cyclosporin A, DeThox LA-23®, and Tea tree oil.

Another embodiment comprises cyclosporin A, DeThox SA-80®, and Tea tree oil.

Another embodiment comprises cyclosporin A, Disponil O5®, and Tea tree oil.

Another embodiment comprises cyclosporin A, Eccoterge EO-41B®, and Tea tree oil.

Another embodiment comprises cyclosporin A, Empilan KA2.5/90®, and Tea tree oil.

Another embodiment comprises cyclosporin A, Empilan KA5/90®, and Tea tree oil.

Another embodiment comprises cyclosporin A, Empilan KM-20®, and Tea tree oil.

Another embodiment comprises cyclosporin A, Empilan KM-50®, and Tea tree oil.

Another embodiment comprises cyclosporin A, Empilan L-23®, and Tea tree oil.

Another embodiment comprises cyclosporin A, Ethylan 25-3®, and Tea tree oil.

Another embodiment comprises cyclosporin A, Ethylan 1204®, and Tea tree oil.

Another embodiment comprises cyclosporin A, Ethylan DA-4®, and Tea tree oil.

Another embodiment comprises cyclosporin A, Ethylan LA-230®, and Tea tree oil.

Another embodiment comprises cyclosporin A, Ethylan SN®, and Tea tree oil.

Another embodiment comprises cyclosporin A, Ethylan TD-60®, and Tea tree oil.

Another embodiment comprises cyclosporin A, Ethylan TD-100®, and Tea tree oil.

Another embodiment comprises cyclosporin A, Ethylan TD-1407®, and Tea tree oil.

Another embodiment comprises cyclosporin A, Eumulgin B1®, and Tea tree oil.

Another embodiment comprises cyclosporin A, Eumulgin B2®, and Tea tree oil.

Another embodiment comprises cyclosporin A, Eumulgin B3®, and Tea tree oil.

Another embodiment comprises cyclosporin A, Eumulgin O-10®, and Tea tree oil.

Another embodiment comprises cyclosporin A, Flo Mo 80/20®, and Tea tree oil.

Another embodiment comprises cyclosporin A, Flo Mo Low Foam®, and Tea tree oil.

Another embodiment comprises cyclosporin A, Forlan C-24®, and Tea tree oil.

Another embodiment comprises cyclosporin A, Genapol 1454®, and Tea tree oil.

Another embodiment comprises cyclosporin A, Genapol BA-020®, and Tea tree oil.

Another embodiment comprises cyclosporin A, Genapol BA-040®, and Tea tree oil.

Another embodiment comprises cyclosporin A, Genapol C-100®, and Tea tree oil.

Another embodiment comprises cyclosporin A, Genapol DA 060®, and Tea tree oil.

Another embodiment comprises cyclosporin A, Genapol HS 020®, and Tea tree oil.

Another embodiment comprises cyclosporin A, Genapol HS 200®, and Tea tree oil.

Another embodiment comprises cyclosporin A, Genapol ID-040®, and Tea tree oil.

Another embodiment comprises cyclosporin A, Genapol ID-060®, and Tea tree oil.

Another embodiment comprises cyclosporin A, Genapol ID-090®, and Tea tree oil.

Another embodiment comprises cyclosporin A, Genapol LA 010®, and Tea tree oil.

Another embodiment comprises cyclosporin A, Genapol LA 020®, and Tea tree oil.

Another embodiment comprises cyclosporin A, Genapol LA 030®, and Tea tree oil.

Another embodiment comprises cyclosporin A, Genapol LA 040®, and Tea tree oil.

Another embodiment comprises cyclosporin A, Genapol LA 050®, and Tea tree oil.

Another embodiment comprises cyclosporin A, Genapol LA 060®, and Tea tree oil.

Another embodiment comprises cyclosporin A, Genapol LA 070®, and Tea tree oil.

Another embodiment comprises cyclosporin A, Genapol LA 070S®, and Tea tree oil.

Another embodiment comprises cyclosporin A, Genapol LA 230®, and Tea tree oil.

Another embodiment comprises cyclosporin A, Genapol O 020®, and Tea tree oil.

Another embodiment comprises cyclosporin A, Genapol O 050®, and Tea tree oil.

Another embodiment comprises cyclosporin A, Genapol O 100®, and Tea tree oil.

Another embodiment comprises cyclosporin A, Genapol O 200®, and Tea tree oil.

Another embodiment comprises cyclosporin A, Genapol SA 030®, and Tea tree oil.

Another embodiment comprises cyclosporin A, Genapol SA 120®, and Tea tree oil.

Another embodiment comprises cyclosporin A, Genapol T-020®, and Tea tree oil.

Another embodiment comprises cyclosporin A, Genapol UD-030®, and Tea tree oil.

Another embodiment comprises cyclosporin A, Genapol UD-050®, and Tea tree oil.

Another embodiment comprises cyclosporin A, Genapol UD-070®, and Tea tree oil.

Another embodiment comprises cyclosporin A, Genapol UD-079®, and Tea tree oil.

Another embodiment comprises cyclosporin A, Genapol UD-080®, and Tea tree oil.

Another embodiment comprises cyclosporin A, Genapol UD-110®, and Tea tree oil.

Another embodiment comprises cyclosporin A, Genapol X 030®, and Tea tree oil.

Another embodiment comprises cyclosporin A, Genapol X 050®, and Tea tree oil.

Another embodiment comprises cyclosporin A, Genapol X 060®, and Tea tree oil.

Another embodiment comprises cyclosporin A, Genapol X 070®, and Tea tree oil.

Another embodiment comprises cyclosporin A, Genapol X 080®, and Tea tree oil.

Another embodiment comprises cyclosporin A, Genapol X 100®, and Tea tree oil.

Another embodiment comprises cyclosporin A, Genapol X159®, and Tea tree oil.

Another embodiment comprises cyclosporin A, Generol 122 E5®, and Tea tree oil.

Another embodiment comprises cyclosporin A, Generol 122 E25®, and Tea tree oil.

Another embodiment comprises cyclosporin A, Hostacerin T-3®, and Tea tree oil.

Another embodiment comprises cyclosporin A, Iconol LF 110®, and Tea tree oil.

Another embodiment comprises cyclosporin A, Incropol CS-20®, and Tea tree oil.

Another embodiment comprises cyclosporin A, Lexemul CS-20®, and Tea tree oil.

Another embodiment comprises cyclosporin A, Liponic EG-1®, and Tea tree oil.

Another embodiment comprises cyclosporin A, Lipowax D®, and Tea tree oil.

Another embodiment comprises cyclosporin A, Lipowax G®, and Tea tree oil.

Another embodiment comprises cyclosporin A, Lipowax NI®, and Tea tree oil.

Another embodiment comprises cyclosporin A, Lipowax P®, and Tea tree oil.

Another embodiment comprises cyclosporin A, Lipowax P-31®, and Tea tree oil.

Another embodiment comprises cyclosporin A, Lipowax PR®, and Tea tree oil.

Another embodiment comprises cyclosporin A, Lumulse CS-20®, and Tea tree oil.

Another embodiment comprises cyclosporin A, Macol CSA-20®, and Tea tree oil.

Another embodiment comprises cyclosporin A, Marlox B 24/50®, and Tea tree oil.

Another embodiment comprises cyclosporin A, Mazawet 77®, and Tea tree oil.

Another embodiment comprises cyclosporin A, Norfox 1713®, and Tea tree oil.

Another embodiment comprises cyclosporin A, Norfox 2579®, and Tea tree oil.

Another embodiment comprises cyclosporin A, Norox Lo Foam®, and Tea tree oil.

Another embodiment comprises cyclosporin A, Promulgen D®, and Tea tree oil.

Another embodiment comprises cyclosporin A, Promulgen G®, and Tea tree oil.

Another embodiment comprises cyclosporin A, Renex 30®, and Tea tree oil.

Another embodiment comprises cyclosporin A, Renex 36®, and Tea tree oil.

Another embodiment comprises cyclosporin A, Rhodasurf A 24®, and Tea tree oil.

Another embodiment comprises cyclosporin A, Rhodasurf AAE-10®, and Tea tree oil.

Another embodiment comprises cyclosporin A, Rhodasurf BEH-25®, and Tea tree oil.

Another embodiment comprises cyclosporin A, Rhodasurf BEH-40®, and Tea tree oil.

Another embodiment comprises cyclosporin A, Rhodasurf DA 530®, and Tea tree oil.

Another embodiment comprises cyclosporin A, Rhodasurf DA 630®, and Tea tree oil.

Another embodiment comprises cyclosporin A, Rhodasurf DA 639®, and Tea tree oil.

Another embodiment comprises cyclosporin A, Rhodasurf LAN-23®, and Tea tree oil.

Another embodiment comprises cyclosporin A, Rhodasurf ON-870®, and Tea tree oil.

Another embodiment comprises cyclosporin A, Rhodasurf ON-877®, and Tea tree oil.

Another embodiment comprises cyclosporin A, Rhodasurf TB-970 FLK®, and Tea tree oil.

Another embodiment comprises cyclosporin A, Ritacet-20®, and Tea tree oil.

Another embodiment comprises cyclosporin A, Ritachol 1000®, and Tea tree oil.

Another embodiment comprises cyclosporin A, Ritachol 2000®, and Tea tree oil.

Another embodiment comprises cyclosporin A, Ritachol 5000®, and Tea tree oil.

Another embodiment comprises cyclosporin A, Ritox 35®, and Tea tree oil.

Another embodiment comprises cyclosporin A, Surfonic DA-4®, and Tea tree oil.

Another embodiment comprises cyclosporin A, Surfonic DA-6®, and Tea tree oil.

Another embodiment comprises cyclosporin A, Surtonic L46-7®, and Tea tree oil.

Another embodiment comprises cyclosporin A, Surfonic POA®, and Tea tree oil.

Another embodiment comprises cyclosporin A, Synthrapol KB®, and Tea tree oil.

Another embodiment comprises cyclosporin A, Teginacid®, and Tea tree oil.

Another embodiment comprises cyclosporin A, Teginacid C®, and Tea tree oil.

Another embodiment comprises cyclosporin A, Tegotens EC 11®, and Tea tree oil.

Another embodiment comprises cyclosporin A, Tinegal NA®, and Tea tree oil.

Another embodiment comprises cyclosporin A, Tomadol 400®, and Tea tree oil.

Another embodiment comprises cyclosporin A, Tomadol 600®, and Tea tree oil.

Another embodiment comprises cyclosporin A, Tomadol 900®, and Tea tree oil.

Another embodiment comprises cyclosporin A, Uniperol O®, and Tea tree oil.

Another embodiment comprises cyclosporin A, Witconol SN Series®, and Tea tree oil.

Another embodiment comprises cyclosporin A, an Ethoxylated Alkylphenol, and Tea tree oil.

Another embodiment comprises cyclosporin A, Antarox LF-222®, and Tea tree oil.

Another embodiment comprises cyclosporin A, Atlox 775®, and Tea tree oil.

Another embodiment comprises cyclosporin A, Caloxylate N-9®, and Tea tree oil.

Another embodiment comprises cyclosporin A, Canasol NF-1000®, and Tea tree oil.

Another embodiment comprises cyclosporin A, Canasol NF-3000®, and Tea tree oil.

Another embodiment comprises cyclosporin A, Canasol NF-3070®, and Tea tree oil.

Another embodiment comprises cyclosporin A, Canasol OF 1670®, and Tea tree oil.

Another embodiment comprises cyclosporin A, Canasol OF 2570®, and Tea tree oil.

Another embodiment comprises cyclosporin A, Canasol OF 4070®, and Tea tree oil.

Another embodiment comprises cyclosporin A, Chemax DNP-8®, and Tea tree oil.

Another embodiment comprises cyclosporin A, Chemax DNP-18®, and Tea tree oil.

Another embodiment comprises cyclosporin A, Chemax DNP-150/50®, and Tea tree oil.

Another embodiment comprises cyclosporin A, DeSonic 1.5N®, and Tea tree oil.

Another embodiment comprises cyclosporin A, DeSonic 4N®, and Tea tree oil.

Another embodiment comprises cyclosporin A, DeSonic 5N®, and Tea tree oil.

Another embodiment comprises cyclosporin A, DeSonic 6D®, and Tea tree oil.

Another embodiment comprises cyclosporin A, DeSonic 6N®, and Tea tree oil.

Another embodiment comprises cyclosporin A, DeSonic 7N®, and Tea tree oil.

Another embodiment comprises cyclosporin A, DeSonic 9N®, and Tea tree oil.

Another embodiment comprises cyclosporin A, DeSonic 10D®, and Tea tree oil.

Another embodiment comprises cyclosporin A, DeSonic 11N®, and Tea tree oil.

Another embodiment comprises cyclosporin A, DeSonic 12N®, and Tea tree oil.

Another embodiment comprises cyclosporin A, DeSonic 15N®, and Tea tree oil.

Another embodiment comprises cyclosporin A, DeSonic 20N®, and Tea tree oil.

Another embodiment comprises cyclosporin A, Eccoscour RC®, and Tea tree oil.

Another embodiment comprises cyclosporin A, Eccoterge EO-100®, and Tea tree oil.

Another embodiment comprises cyclosporin A, Emulsifier 632/90%®, and Tea tree oil.

Another embodiment comprises cyclosporin A, Geronol AG-821®, and Tea tree oil.

Another embodiment comprises cyclosporin A, Gradonic N-95®, and Tea tree oil.

Another embodiment comprises cyclosporin A, Hetoxide NP-4®, and Tea tree oil.

Another embodiment comprises cyclosporin A, Hetoxide NP-30®, and Tea tree oil.

Another embodiment comprises cyclosporin A, Hostapal N-100®, and Tea tree oil.

Another embodiment comprises cyclosporin A, Hostapal N-110®, and Tea tree oil.

Another embodiment comprises cyclosporin A, Igepal CTA-639W®, and Tea tree oil.

Another embodiment comprises cyclosporin A, Igepal DAP-9®, and Tea tree oil.

Another embodiment comprises cyclosporin A, Igepal OD-410®, and Tea tree oil.

Another embodiment comprises cyclosporin A, Igepal SS-837®, and Tea tree oil.

Another embodiment comprises cyclosporin A, Lipocol NP-9 USP®, and Tea tree oil.

Another embodiment comprises cyclosporin A, Macol DNP-10®, and Tea tree oil.

Another embodiment comprises cyclosporin A, Marlophen NP 5®, and Tea tree oil.

Another embodiment comprises cyclosporin A, Marlophen P 1®, and Tea tree oil.

Another embodiment comprises cyclosporin A, Surfonic NB®, and Tea tree oil.

Another embodiment comprises cyclosporin A, Surfonic OPB-307®, and Tea tree oil.

Another embodiment comprises cyclosporin A, Surfonic OPB-407®, and Tea tree oil.

Another embodiment comprises cyclosporin A, Syn Fac 334®, and Tea tree oil.

Another embodiment comprises cyclosporin A, Syn Fac 8216®, and Tea tree oil.

Another embodiment comprises cyclosporin A, Triton N-57®, and Tea tree oil.

Another embodiment comprises cyclosporin A, Trycol 6956®, and Tea tree oil.

Another embodiment comprises cyclosporin A, Trycol 6961®, and Tea tree oil.

Another embodiment comprises cyclosporin A, Trycol 6964®, and Tea tree oil.

Another embodiment comprises cyclosporin A, Trycol 6969®, and Tea tree oil.

Another embodiment comprises cyclosporin A, Trycol 6974®, and Tea tree oil.

Another embodiment comprises cyclosporin A, Witbreak DRB-127®, and Tea tree oil.

Another embodiment comprises cyclosporin A, Witbreak DRB-127®, and Tea tree oil.

Another embodiment comprises cyclosporin A, Witconol NP Series®, and Tea tree oil.

Another embodiment comprises cyclosporin A, an Ethoxylated Aryl Phenol, and Tea tree oil.

Another embodiment comprises cyclosporin A, Soprophor BSU®, and Tea tree oil.

Another embodiment comprises cyclosporin A, Soprophor CY/8®, and Tea tree oil.

Another embodiment comprises cyclosporin A, Soprophor S/25®, and Tea tree oil.

Another embodiment comprises cyclosporin A, Witconol NIO®, and Tea tree oil.

Another embodiment comprises cyclosporin A, Witconol NIW®, and Tea tree oil.

Another embodiment comprises cyclosporin A, Witconol S-100®, and Tea tree oil.

Another embodiment comprises cyclosporin A, an Ethoxylated Fatty Acid, and Tea tree oil.

Another embodiment comprises cyclosporin A, Aldo PGHMS®, and Tea tree oil.

Another embodiment comprises cyclosporin A, Alkamuls TO-15/HR®, and Tea tree oil.

Another embodiment comprises cyclosporin A, Armotan AL-69-66®, and Tea tree oil.

Another embodiment comprises cyclosporin A, Cerasynt 840®, and Tea tree oil.

Another embodiment comprises cyclosporin A, Cerasynt 945®, and Tea tree oil.

Another embodiment comprises cyclosporin A, Crystal Inhibitor No. 5®, and Tea tree oil.

Another embodiment comprises cyclosporin A, DeThox Acid L-9®, and Tea tree oil.

Another embodiment comprises cyclosporin A, DeThox Acid S-8®, and Tea tree oil.

Another embodiment comprises cyclosporin A, Ethofat 242/25®, and Tea tree oil.

Another embodiment comprises cyclosporin A, Hydropalat 65®, and Tea tree oil.

Another embodiment comprises cyclosporin A, Lipo EGMS®, and Tea tree oil.

Another embodiment comprises cyclosporin A, Lipopeg 2 DL®, and Tea tree oil.

Another embodiment comprises cyclosporin A, Lipopeg 4 DL®, and Tea tree oil.

Another embodiment comprises cyclosporin A, Lipopeg 4-L®, and Tea tree oil.

Another embodiment comprises cyclosporin A, Lipopeg 39-S®, and Tea tree oil.

Another embodiment comprises cyclosporin A, Lipopeg 4-S®, and Tea tree oil.

Another embodiment comprises cyclosporin A, Lipopeg 10-S®, and Tea tree oil.

Another embodiment comprises cyclosporin A, Lipopeg 100-S®, and Tea tree oil.

Another embodiment comprises cyclosporin A, Lipopeg 6000 DS®, and Tea tree oil.

Another embodiment comprises cyclosporin A, Lumulse 40-L®, and Tea tree oil.

Another embodiment comprises cyclosporin A, Lumulse 40-S®, and Tea tree oil.

Another embodiment comprises cyclosporin A, Lumulse 42-L®, and Tea tree oil.

Another embodiment comprises cyclosporin A, Lumulse 42-S®, and Tea tree oil.

Another embodiment comprises cyclosporin A, Lumulse 100-S®, and Tea tree oil.

Another embodiment comprises cyclosporin A, Lumulse 602-S®, and Tea tree oil.

Another embodiment comprises cyclosporin A, Magrabar PGE-20-0®, and Tea tree oil.

Another embodiment comprises cyclosporin A, Magrabar PGE-20L®, and Tea tree oil.

Another embodiment comprises cyclosporin A, Magrabar PGE-20T®, and Tea tree oil.

Another embodiment comprises cyclosporin A, Magrabar PGE-22-0®, and Tea tree oil.

Another embodiment comprises cyclosporin A, Magrabar PGE-22L®, and Tea tree oil.

Another embodiment comprises cyclosporin A, Magrabar PGE-22T®, and Tea tree oil.

Another embodiment comprises cyclosporin A, Magrabar PGE-40-0®, and Tea tree oil.

Another embodiment comprises cyclosporin A, Magrabar PGE-40L®, and Tea tree oil.

Another embodiment comprises cyclosporin A, Magrabar PGE-40T®, and Tea tree oil.

Another embodiment comprises cyclosporin A, Magrabar PGE-42-0®, and Tea tree oil.

Another embodiment comprises cyclosporin A, Magrabar PGE-42L®, and Tea tree oil.

Another embodiment comprises cyclosporin A, Magrabar PGE-42T®, and Tea tree oil.

Another embodiment comprises cyclosporin A, Magrabar PGE-60-0®, and Tea tree oil.

Another embodiment comprises cyclosporin A, Magrabar PGE-60L®, and Tea tree oil.

Another embodiment comprises cyclosporin A, Magrabar PGE-60T®, and Tea tree oil.

Another embodiment comprises cyclosporin A, Magrabar PGE-62-0®, and Tea tree oil.

Another embodiment comprises cyclosporin A, Magrabar PGE-62L®, and Tea tree oil.

Another embodiment comprises cyclosporin A, Magrabar PGE-62T®, and Tea tree oil.

Another embodiment comprises cyclosporin A, Mapeg S-40K®, and Tea tree oil.

Another embodiment comprises cyclosporin A, Marlowet OTS®, and Tea tree oil.

Another embodiment comprises cyclosporin A, Naturechem PGR®, and Tea tree oil.

Another embodiment comprises cyclosporin A, PG No. 4®, and Tea tree oil.

Another embodiment comprises cyclosporin A, Renex 20®, and Tea tree oil.

Another embodiment comprises cyclosporin A, Ritox 52®, and Tea tree oil.

Another embodiment comprises cyclosporin A, Ritox 53®, and Tea tree oil.

Another embodiment comprises cyclosporin A, Ritox 59®, and Tea tree oil.

Another embodiment comprises cyclosporin A, Surfax 8916/A®, and Tea tree oil.

Another embodiment comprises cyclosporin A, Tego Acid S 40 P®, and Tea tree oil.

Another embodiment comprises cyclosporin A, Tego Acid S 100 P®, and Tea tree oil.

Another embodiment comprises cyclosporin A, Tween 20®, and Tea tree oil.

Another embodiment comprises cyclosporin A, Volpo 131®, and Tea tree oil.

Another embodiment comprises cyclosporin A, an Ethoxylated Fatty Ester or Oil, and Tea tree oil.

Another embodiment comprises cyclosporin A, Acconon 6-C10®, and Tea tree oil.

Another embodiment comprises cyclosporin A, Acconon CC-6®, and Tea tree oil.

Another embodiment comprises cyclosporin A, Acconon CO-7®, and Tea tree oil.

Another embodiment comprises cyclosporin A, Aldosperse 40/60 FG®, and Tea tree oil.

Another embodiment comprises cyclosporin A, Aldosperse ML-23®, and Tea tree oil.

Another embodiment comprises cyclosporin A, Aldosperse MS-20 FG®, and Tea tree oil.

Another embodiment comprises cyclosporin A, Alkamuls EL-620®, and Tea tree oil.

Another embodiment comprises cyclosporin A, Alkamuls EL-719®, and Tea tree oil.

Another embodiment comprises cyclosporin A, Alkamuls EL-985®, and Tea tree oil.

Another embodiment comprises cyclosporin A, Arlatone G®, and Tea tree oil.

Another embodiment comprises cyclosporin A, Arlatone T®, and Tea tree oil.

Another embodiment comprises cyclosporin A, Atlas G-1045A®, and Tea tree oil.

Another embodiment comprises cyclosporin A, Atlas G-1086®, and Tea tree oil.

Another embodiment comprises cyclosporin A, Atlas G-1087®, and Tea tree oil.

Another embodiment comprises cyclosporin A, Atlas G-1089®, and Tea tree oil.

Another embodiment comprises cyclosporin A, Atlas G-1096®, and Tea tree oil.

Another embodiment comprises cyclosporin A, Atlas G-1292®, and Tea tree oil.

Another embodiment comprises cyclosporin A, Atlas G-1293®, and Tea tree oil.

Another embodiment comprises cyclosporin A, Atlas G-1300®, and Tea tree oil.

Another embodiment comprises cyclosporin A, Atlas G-7076®, and Tea tree oil.

Another embodiment comprises cyclosporin A, Capmul EMG®, and Tea tree oil.

Another embodiment comprises cyclosporin A, Chemonic CO-40®, and Tea tree oil.

Another embodiment comprises cyclosporin A, Chemonic LI-3®, and Tea tree oil.

Another embodiment comprises cyclosporin A, Chemonic LI-7®, and Tea tree oil.

Another embodiment comprises cyclosporin A, Cirrasol GM®, and Tea tree oil.

Another embodiment comprises cyclosporin A, Cremophor CO 40®, and Tea tree oil.

Another embodiment comprises cyclosporin A, Cremophor CO 410®, and Tea tree oil.

Another embodiment comprises cyclosporin A, Cremophor EL®, and Tea tree oil.

Another embodiment comprises cyclosporin A, Cremophor GC7®, and Tea tree oil.

Another embodiment comprises cyclosporin A, Cremophor RH-40®, and Tea tree oil.

Another embodiment comprises cyclosporin A, Crovol A-40®, and Tea tree oil.

Another embodiment comprises cyclosporin A, Crovol A-70®, and Tea tree oil.

Another embodiment comprises cyclosporin A, Crovol M-70®, and Tea tree oil.

Another embodiment comprises cyclosporin A, Crovol PK-70®, and Tea tree oil.

Another embodiment comprises cyclosporin A, Cutina E-24®, and Tea tree oil.

Another embodiment comprises cyclosporin A, Dacospin 12-R®, and Tea tree oil.

Another embodiment comprises cyclosporin A, Dehymuls HRE-7®, and Tea tree oil.

Another embodiment comprises cyclosporin A, DeSonic 30C®, and Tea tree oil.

Another embodiment comprises cyclosporin A, DeSonic 36C®, and Tea tree oil.

Another embodiment comprises cyclosporin A, DeSonic 40C®, and Tea tree oil.

Another embodiment comprises cyclosporin A, Durfax 60®, and Tea tree oil.

Another embodiment comprises cyclosporin A, Durfax 65®, and Tea tree oil.

Another embodiment comprises cyclosporin A, Durfax 80®, and Tea tree oil.

Another embodiment comprises cyclosporin A, Durfax EOM®, and Tea tree oil.

Another embodiment comprises cyclosporin A, Eccoterge NF-2®, and Tea tree oil.

Another embodiment comprises cyclosporin A, Emulpon CO-360®, and Tea tree oil.

Another embodiment comprises cyclosporin A, Emulpon CO-550®, and Tea tree oil.

Another embodiment comprises cyclosporin A, Emulsogen EL®, and Tea tree oil.

Another embodiment comprises cyclosporin A, Emulsogen HCO 040®, and Tea tree oil.

Another embodiment comprises cyclosporin A, Emulsogen HCO 060®, and Tea tree oil.

Another embodiment comprises cyclosporin A, Emulsynt 1055®, and Tea tree oil.

Another embodiment comprises cyclosporin A, Ethox 3095®, and Tea tree oil.

Another embodiment comprises cyclosporin A, Eumulgin RO-40®, and Tea tree oil.

Another embodiment comprises cyclosporin A, Genapol G-260®, and Tea tree oil.

Another embodiment comprises cyclosporin A, Glycosperse L-20®, and Tea tree oil.

Another embodiment comprises cyclosporin A, Glycosperse O-5®, and Tea tree oil.

Another embodiment comprises cyclosporin A, Glycosperse O-20®, and Tea tree oil.

Another embodiment comprises cyclosporin A, Glycosperse O-20 FG®, and Tea tree oil.

Another embodiment comprises cyclosporin A, Glycosperse S-20®, and Tea tree oil.

Another embodiment comprises cyclosporin A, Glycosperse S-20 FG®, and Tea tree oil.

Another embodiment comprises cyclosporin A, Glycosperse TS-20®, and Tea tree oil.

Another embodiment comprises cyclosporin A, Glycosperse TS-20 FG®, and Tea tree oil.

Another embodiment comprises cyclosporin A, Hetan SL®, and Tea tree oil.

Another embodiment comprises cyclosporin A, Hetan SO®, and Tea tree oil.

Another embodiment comprises cyclosporin A, Hetan SS®, and Tea tree oil.

Another embodiment comprises cyclosporin A, Hetoxide C-2®, and Tea tree oil.

Another embodiment comprises cyclosporin A, Hetoxide C-9®, and Tea tree oil.

Another embodiment comprises cyclosporin A, Hetoxide C-15®, and Tea tree oil.

Another embodiment comprises cyclosporin A, Hetoxide C-25®, and Tea tree oil.

Another embodiment comprises cyclosporin A, Hetoxide C-40®, and Tea tree oil.

Another embodiment comprises cyclosporin A, Hetoxide C-200®, and Tea tree oil.

Another embodiment comprises cyclosporin A, Hetoxide C-200-50%®, and Tea tree oil.

Another embodiment comprises cyclosporin A, Hetoxide GC-30®, and Tea tree oil.

Another embodiment comprises cyclosporin A, Hetoxide HC-60®, and Tea tree oil.

Another embodiment comprises cyclosporin A, Ice No. 2®, and Tea tree oil.

Another embodiment comprises cyclosporin A, Incrocas 30/40®, and Tea tree oil.

Another embodiment comprises cyclosporin A, Lexol EC®, and Tea tree oil.

Another embodiment comprises cyclosporin A, Lexol EO®, and Tea tree oil.

Another embodiment comprises cyclosporin A, Lipocol HCO-40®, and Tea tree oil.

Another embodiment comprises cyclosporin A, Lipocol HCO-60®, and Tea tree oil.

Another embodiment comprises cyclosporin A, Lipocol O-3 Special®, and Tea tree oil.

Another embodiment comprises cyclosporin A, Lipopeg 2-L®, and Tea tree oil.

Another embodiment comprises cyclosporin A, Lipopeg 4-DO®, and Tea tree oil.

Another embodiment comprises cyclosporin A, Lipopeg 4-DS®, and Tea tree oil.

Another embodiment comprises cyclosporin A, Lipovol GTB®, and Tea tree oil.

Another embodiment comprises cyclosporin A, Lonzest SML-20®, and Tea tree oil.

Another embodiment comprises cyclosporin A, Lonzest SMO-20®, and Tea tree oil.

Another embodiment comprises cyclosporin A, Lonzest SMS-20®, and Tea tree oil.

Another embodiment comprises cyclosporin A, Lonzest STO-20®, and Tea tree oil.

Another embodiment comprises cyclosporin A, Lonzest STS-20®, and Tea tree oil.

Another embodiment comprises cyclosporin A, Lumulse GR-40®, and Tea tree oil.

Another embodiment comprises cyclosporin A, Lumulse GRH-40®, and Tea tree oil.

Another embodiment comprises cyclosporin A, Lumulse POE (7) GML®, and Tea tree oil.

Another embodiment comprises cyclosporin A, Lumulse POE (12) Glyc®, and Tea tree oil.

Another embodiment comprises cyclosporin A, Lumulse POE (40) MS KP®, and Tea tree oil.

Another embodiment comprises cyclosporin A, Marlowet 4750®, and Tea tree oil.

Another embodiment comprises cyclosporin A, Marlowet LVS®, and Tea tree oil.

Another embodiment comprises cyclosporin A, Marlowet R 11®, and Tea tree oil.

Another embodiment comprises cyclosporin A, Marlowet R 40®, and Tea tree oil.

Another embodiment comprises cyclosporin A, Mazol 80 MGK®, and Tea tree oil.

Another embodiment comprises cyclosporin A, Nonionic Emulsifier T-9®, and Tea tree oil.

Another embodiment comprises cyclosporin A, Oronal LCG®, and Tea tree oil.

Another embodiment comprises cyclosporin A, Polyderm PPI-CO-200®, and Tea tree oil.

Another embodiment comprises cyclosporin A, Polyderm PPI-CO-40®, and Tea tree oil.

Another embodiment comprises cyclosporin A, Rewoderm LI 520-70®, and Tea tree oil.

Another embodiment comprises cyclosporin A, Ritapeg 150 DS®, and Tea tree oil.

Another embodiment comprises cyclosporin A, Softigen 767®, and Tea tree oil.

Another embodiment comprises cyclosporin A, Surfactol 318®, and Tea tree oil.

Another embodiment comprises cyclosporin A, Surfactol 365®, and Tea tree oil.

Another embodiment comprises cyclosporin A, Syn Lube 107®, and Tea tree oil.

Another embodiment comprises cyclosporin A, Syn Lube 728®, and Tea tree oil.

Another embodiment comprises cyclosporin A, Syn Lube 1632H®, and Tea tree oil.

Another embodiment comprises cyclosporin A, Syn Lube 6277-A®, and Tea tree oil.

Another embodiment comprises cyclosporin A, T-Det C-20®, and Tea tree oil.

Another embodiment comprises cyclosporin A, T-Det C-40®, and Tea tree oil.

Another embodiment comprises cyclosporin A, Tally 100 Plus®, and Tea tree oil.

Another embodiment comprises cyclosporin A, Uniperol EL®, and Tea tree oil.

Another embodiment comprises cyclosporin A, a Fatty Ester, and Tea tree oil.

Another embodiment comprises cyclosporin A, Actralube-Syn 147®, and Tea tree oil.

Another embodiment comprises cyclosporin A, Atlas G-1556®, and Tea tree oil.

Another embodiment comprises cyclosporin A, Atlas G-1564®, and Tea tree oil.

Another embodiment comprises cyclosporin A, Atlasol Base Oil S®, and Tea tree oil.

Another embodiment comprises cyclosporin A, Base ML®, and Tea tree oil.

Another embodiment comprises cyclosporin A, Base MT®, and Tea tree oil.

Another embodiment comprises cyclosporin A, Cerasynt 303®, and Tea tree oil.

Another embodiment comprises cyclosporin A, Dermol 1012®, and Tea tree oil.

Another embodiment comprises cyclosporin A, Kemester 4000®, and Tea tree oil.

Another embodiment comprises cyclosporin A, Lactipol S®, and Tea tree oil.

Another embodiment comprises cyclosporin A, Magrabar PGO®, and Tea tree oil.

Another embodiment comprises cyclosporin A, Mayco Base BFO®, and Tea tree oil.

Another embodiment comprises cyclosporin A, Methyl Linoleate®, and Tea tree oil.

Another embodiment comprises cyclosporin A, Pationic 122A®, and Tea tree oil.

Another embodiment comprises cyclosporin A, Pationic 138C®, and Tea tree oil.

Another embodiment comprises cyclosporin A, Pationic CSL®, and Tea tree oil.

Another embodiment comprises cyclosporin A, Pationic ISL®, and Tea tree oil.

Another embodiment comprises cyclosporin A, Pationic SBL®, and Tea tree oil.

Another embodiment comprises cyclosporin A, Pationic SSL®, and Tea tree oil.

Another embodiment comprises cyclosporin A, Ritasol®, and Tea tree oil.

Another embodiment comprises cyclosporin A, Tego Alkanol CS 20®, and Tea tree oil.

Another embodiment comprises cyclosporin A, Tego Alkanol L23 P®, and Tea tree oil.

Another embodiment comprises cyclosporin A, Tego Alkanol S2®, and Tea tree oil.

Another embodiment comprises cyclosporin A, Tego Alkanol S20 P®, and Tea tree oil.

Another embodiment comprises cyclosporin A, Triemulsifier 600 MS®, and Tea tree oil.

Another embodiment comprises cyclosporin A, a Glycerol Esters, and Tea tree oil.

Another embodiment comprises cyclosporin A, Agro #9 Wint SBO®, and Tea tree oil.

Another embodiment comprises cyclosporin A, Ahcovel Base 700®, and Tea tree oil.

Another embodiment comprises cyclosporin A, Aldo HMS FG®, and Tea tree oil.

Another embodiment comprises cyclosporin A, Aldo MLD®, and Tea tree oil.

Another embodiment comprises cyclosporin A, Aldo MLD FG®, and Tea tree oil.

Another embodiment comprises cyclosporin A, Aldo MO FG®, and Tea tree oil.

Another embodiment comprises cyclosporin A, Aldo MS®, and Tea tree oil.

Another embodiment comprises cyclosporin A, Aldo MS FG®, and Tea tree oil.

Another embodiment comprises cyclosporin A, Aldo MS LG FG®, and Tea tree oil.

Another embodiment comprises cyclosporin A, Aldo MSD®, and Tea tree oil.

Another embodiment comprises cyclosporin A, Aldo MSD FG®, and Tea tree oil.

Another embodiment comprises cyclosporin A, Aldosperse O-20 FG®, and Tea tree oil.

Another embodiment comprises cyclosporin A, Aldosperse TS-20 FG®, and Tea tree oil.

Another embodiment comprises cyclosporin A, Aldosperse TS-40 FG®, and Tea tree oil.

Another embodiment comprises cyclosporin A, Arlacel 165®, and Tea tree oil.

Another embodiment comprises cyclosporin A, Arlacel 186®, and Tea tree oil.

Another embodiment comprises cyclosporin A, Capmul GMO®, and Tea tree oil.

Another embodiment comprises cyclosporin A, Capmul GMS®, and Tea tree oil.

Another embodiment comprises cyclosporin A, Caprol 3GO®, and Tea tree oil.

Another embodiment comprises cyclosporin A, Caprol 3GVS®, and Tea tree oil.

Another embodiment comprises cyclosporin A, Caprol 6G2S®, and Tea tree oil.

Another embodiment comprises cyclosporin A, Caprol 10G40®, and Tea tree oil.

Another embodiment comprises cyclosporin A, Caprol 10G100®, and Tea tree oil.

Another embodiment comprises cyclosporin A, Caprol ET®, and Tea tree oil.

Another embodiment comprises cyclosporin A, Caprol PGE860®, and Tea tree oil.

Another embodiment comprises cyclosporin A, Cerasynt 945®, and Tea tree oil.

Another embodiment comprises cyclosporin A, Cerasynt GMS®, and Tea tree oil.

Another embodiment comprises cyclosporin A, Cerasynt Q®, and Tea tree oil.

Another embodiment comprises cyclosporin A, Cerasynt SD®, and Tea tree oil.

Another embodiment comprises cyclosporin A, Cerasynt WM®, and Tea tree oil.

Another embodiment comprises cyclosporin A, Chemsperse 14®, and Tea tree oil.

Another embodiment comprises cyclosporin A, Cremophor GO-32®, and Tea tree oil.

Another embodiment comprises cyclosporin A, Cremophor GS11®, and Tea tree oil.

Another embodiment comprises cyclosporin A, Cremophor GS-32®, and Tea tree oil.

Another embodiment comprises cyclosporin A, Cutina KD-16®, and Tea tree oil.

Another embodiment comprises cyclosporin A, Dehymuls PGPH®, and Tea tree oil.

Another embodiment comprises cyclosporin A, Dermol DGDIS®, and Tea tree oil.

Another embodiment comprises cyclosporin A, Dermol DGMIS®, and Tea tree oil.

Another embodiment comprises cyclosporin A, Dermol G-76®, and Tea tree oil.

Another embodiment comprises cyclosporin A, Dermol G-7DI®, and Tea tree oil.

Another embodiment comprises cyclosporin A, Dermol NGDI®, and Tea tree oil.

Another embodiment comprises cyclosporin A, Dermolan GLH®, and tea tree oil.

Another embodiment comprises cyclosporin A, Drewmulse GMO®, and Tea tree oil.

Another embodiment comprises cyclosporin A, Drewpol 3-5-M®, and Tea tree oil.

Another embodiment comprises cyclosporin A, Durlac 100 W®, and Tea tree oil.

Another embodiment comprises cyclosporin A, Dur-Lo®, and Tea tree oil.

Another embodiment comprises cyclosporin A, Dynasan 118®, and Tea tree oil.

Another embodiment comprises cyclosporin A, EC-25®, and Tea tree oil.

Another embodiment comprises cyclosporin A, EM 40®, and Tea tree oil.

Another embodiment comprises cyclosporin A, Emerest 2400®, and Tea tree oil.

Another embodiment comprises cyclosporin A, Emerest 2452®, and Tea tree oil.

Another embodiment comprises cyclosporin A, Empilan G-26®, and Tea tree oil.

Another embodiment comprises cyclosporin A, Genapol TSM®, and Tea tree oil.

Another embodiment comprises cyclosporin A, Hostacerin DGI®, and Tea tree oil.

Another embodiment comprises cyclosporin A, Hostacerin DGL®, and Tea tree oil.

Another embodiment comprises cyclosporin A, Hostacerin DGMS®, and Tea tree oil.

Another embodiment comprises cyclosporin A, Hostacerin DGSB®, and Tea tree oil.

Another embodiment comprises cyclosporin A, Ice No. 2®, and Tea tree oil.

Another embodiment comprises cyclosporin A, Imwitor 742®, and Tea tree oil.

Another embodiment comprises cyclosporin A, Imwitor 780 K®, and Tea tree oil.

Another embodiment comprises cyclosporin A, Imwitor 960 Flakes®, and Tea tree oil.

Another embodiment comprises cyclosporin A, Isolan GI 34®, and Tea tree oil.

Another embodiment comprises cyclosporin A, Isolan GO 33®, and Tea tree oil.

Another embodiment comprises cyclosporin A, Kemester 1000®, and Tea tree oil.

Another embodiment comprises cyclosporin A, Kemester 2000®, and Tea tree oil.

Another embodiment comprises cyclosporin A, Kemester 2000®, and Tea tree oil.

Another embodiment comprises cyclosporin A, Kemester 6000SE®, and Tea tree oil.

Another embodiment comprises cyclosporin A, Lamecreme DGE 18®, and Tea tree oil.

Another embodiment comprises cyclosporin A, Lexemul 515®, and Tea tree oil.

Another embodiment comprises cyclosporin A, Lexemul 561®, and Tea tree oil.

Another embodiment comprises cyclosporin A, Lexemul AR®, and Tea tree oil.

Another embodiment comprises cyclosporin A, Lexemul AS®, and Tea tree oil.

Another embodiment comprises cyclosporin A, Lexemul GDL®, and Tea tree oil.

Another embodiment comprises cyclosporin A, Lexemul T®, and Tea tree oil.

Another embodiment comprises cyclosporin A, Lipomulse 165®, and Tea tree oil.

Another embodiment comprises cyclosporin A, Lumulse GML K®, and Tea tree oil.

Another embodiment comprises cyclosporin A, Lumulse GMO K®, and Tea tree oil.

Another embodiment comprises cyclosporin A, Lumulse GMR K®, and Tea tree oil.

Another embodiment comprises cyclosporin A, Lumulse GMT K®, and Tea tree oil.

Another embodiment comprises cyclosporin A, Magrabar GMC®, and Tea tree oil.

Another embodiment comprises cyclosporin A, Magrabar GMO-CK®, and Tea tree oil.

Another embodiment comprises cyclosporin A, Magrabar MDG-5050®, and Tea tree oil.

Another embodiment comprises cyclosporin A, Magrabar PGO-315®, and Tea tree oil.

Another embodiment comprises cyclosporin A, Magrabar PGO-1010®, and Tea tree oil.

Another embodiment comprises cyclosporin A, Mazol 300K®, and Tea tree oil.

Another embodiment comprises cyclosporin A, Mazol GMO-K®, and Tea tree oil.

Another embodiment comprises cyclosporin A, Mazol GMS-K®, and Tea tree oil.

Another embodiment comprises cyclosporin A, Mazol PG031-K®, and Tea tree oil.

Another embodiment comprises cyclosporin A, Miglyol 812®, and Tea tree oil.

Another embodiment comprises cyclosporin A, Norfox 165C®, and Tea tree oil.

Another embodiment comprises cyclosporin A, Schercemol GMIS®, and Tea tree oil.

Another embodiment comprises cyclosporin A, Tegin®, and Tea tree oil.

Another embodiment comprises cyclosporin A, Tegin 4100 Pellets®, and Tea tree oil.

Another embodiment comprises cyclosporin A, Tegin M Pellets®, and Tea tree oil.

Another embodiment comprises cyclosporin A, Tegin OV®, and Tea tree oil.

Another embodiment comprises cyclosporin A, Teginacid H®, and Tea tree oil.

Another embodiment comprises cyclosporin A, Tego Cosmo P813®, and Tea tree oil.

Another embodiment comprises cyclosporin A, Wickenol 535®, and Tea tree oil.

Another embodiment comprises cyclosporin A, Witconol 14®, and Tea tree oil.

Another embodiment comprises cyclosporin A, Witconol 14®, and Tea tree oil.

Another embodiment comprises cyclosporin A, Witconol 14F®, and Tea tree oil.

Another embodiment comprises cyclosporin A, Witconol 18L®, and Tea tree oil.

Another embodiment comprises cyclosporin A, Witconol GOT®, and Tea tree oil.

Another embodiment comprises cyclosporin A, Witconol MST®, and Tea tree oil.

Another embodiment comprises cyclosporin A, Witconol RHT®, and Tea tree oil.

Another embodiment comprises cyclosporin A, a Glycol Ester, and Tea tree oil.

Another embodiment comprises cyclosporin A, Alkamuls 600 DO®, and Tea tree oil.

Another embodiment comprises cyclosporin A, Alkamuls SEG®, and Tea tree oil.

Another embodiment comprises cyclosporin A, Atlas EM-2®, and Tea tree oil.

Another embodiment comprises cyclosporin A, Cerasynt IP®, and Tea tree oil.

Another embodiment comprises cyclosporin A, Cerasynt M®, and Tea tree oil.

Another embodiment comprises cyclosporin A, Cerasynt MN®, and Tea tree oil.

Another embodiment comprises cyclosporin A, Cerasynt PA®, and Tea tree oil.

Another embodiment comprises cyclosporin A, Chemsperse EGDS®, and Tea tree oil.

Another embodiment comprises cyclosporin A, Chemsperse EGMS®, and Tea tree oil.

Another embodiment comprises cyclosporin A, Colonial Monolaurin®, and Tea tree oil.

Another embodiment comprises cyclosporin A, DeMuls SGE-95®, and Tea tree oil.

Another embodiment comprises cyclosporin A, Eccoterge 200®, and Tea tree oil.

Another embodiment comprises cyclosporin A, Emerest 2380®, and Tea tree oil.

Another embodiment comprises cyclosporin A, Ethox 2610®, and Tea tree oil.

Another embodiment comprises cyclosporin A, Ethox DO-9®, and Tea tree oil.

Another embodiment comprises cyclosporin A, Ethox DO-14®, and Tea tree oil.

Another embodiment comprises cyclosporin A, Ethox SO-9®, and Tea tree oil.

Another embodiment comprises cyclosporin A, Fizul MD-318®, and Tea tree oil.

Another embodiment comprises cyclosporin A, Genapol EGDS-VHP®, and Tea tree oil.

Another embodiment comprises cyclosporin A, Genapol TS Powder®, and Tea tree oil.

Another embodiment comprises cyclosporin A, Hostacerin WO®, and Tea tree oil.

Another embodiment comprises cyclosporin A, Inversol 140®, and Tea tree oil.

Another embodiment comprises cyclosporin A, Kemester 104®, and Tea tree oil.

Another embodiment comprises cyclosporin A, Kemester 205®, and Tea tree oil.

Another embodiment comprises cyclosporin A, Kemester 226®, and Tea tree oil.

Another embodiment comprises cyclosporin A, Kemester 5221SE®, and Tea tree oil.

Another embodiment comprises cyclosporin A, Kemester EGDS®, and Tea tree oil.

Another embodiment comprises cyclosporin A, Lexemul EGDS®, and Tea tree oil.

Another embodiment comprises cyclosporin A, Lexemul EGMS®, and Tea tree oil.

Another embodiment comprises cyclosporin A, Lexemul P®, and Tea tree oil.

Another embodiment comprises cyclosporin A, Lipo DGLS®, and Tea tree oil.

Another embodiment comprises cyclosporin A, Lipo EGDS®, and Tea tree oil.

Another embodiment comprises cyclosporin A, Lipo PGMS®, and Tea tree oil.

Another embodiment comprises cyclosporin A, Liposorb S-4®, and Tea tree oil.

Another embodiment comprises cyclosporin A, Liposorb TO-20®, and Tea tree oil.

Another embodiment comprises cyclosporin A, Lumulse PGO®, and Tea tree oil.

Another embodiment comprises cyclbsporin A, Mackester EGDS®, and Tea tree oil.

Another embodiment comprises cyclosporin A, Mackester EGMS®, and Tea tree oil.

Another embodiment comprises cyclosporin A, Mackester GSTP®, and Tea tree oil.

Another embodiment comprises cyclosporin A, Mackester Series®, and Tea tree oil.

Another embodiment comprises cyclosporin A, Magrabar PDG-50®, and Tea tree oil.

Another embodiment comprises cyclosporin A, Mapeg 6000 DS®, and Tea tree oil.

Another embodiment comprises cyclosporin A, Marlowet 4702®, and Tea tree oil.

Another embodiment comprises cyclosporin A, Monalube 305®, and Tea tree oil.

Another embodiment comprises cyclosporin A, Monalube 310®, and Tea tree oil.

Another embodiment comprises cyclosporin A, Monalube 315®, and Tea tree oil.

Another embodiment comprises cyclosporin A, Monalube 320®, and Tea tree oil.

Another embodiment comprises cyclosporin A, Monalube 325®, and Tea tree oil.

Another embodiment comprises cyclosporin A, Monalube 330®, and Tea tree oil.

Another embodiment comprises cyclosporin A, Naturechem PGHS®, and Tea tree oil.

Another embodiment comprises cyclosporin A, Polycastorol PLO-840®, and Tea tree oil.

Another embodiment comprises cyclosporin A, Polytex 10M®, and Tea tree oil.

Another embodiment comprises cyclosporin A, Ritasynt IP®, and Tea tree oil.

Another embodiment comprises cyclosporin A, Ross Chem PEG 600 DT®, and Tea tree oil.

Another embodiment comprises cyclosporin A, Schercemol PGMS®, and Tea tree oil.

Another embodiment comprises cyclosporin A, Sponto H-44C®, and Tea tree oil.

Another embodiment comprises cyclosporin A, Tegin G®, and Tea tree oil.

Another embodiment comprises cyclosporin A, Witbreak DGE-182®, and Tea tree oil.

Another embodiment comprises cyclosporin A, Witbreak DGE-182®, and Tea tree oil.

Another embodiment comprises cyclosporin A, Witbreak DRA-21®, and Tea tree oil.

Another embodiment comprises cyclosporin A, Witbreak DRA-21®, and Tea tree oil.

Another embodiment comprises cyclosporin A, Witbreak DRA-50®, and Tea tree oil.

Another embodiment comprises cyclosporin A, Witbreak DRA-50®, and Tea tree oil.

Another embodiment comprises cyclosporin A, Witconol F26-46®, and Tea tree oil.

Another embodiment comprises cyclosporin A, Witconol H-32®, and Tea tree oil.

Another embodiment comprises cyclosporin A, Witconol H-33®, and Tea tree oil.

Another embodiment comprises cyclosporin A, Witconol H-35A®, and Tea tree oil.

Another embodiment comprises cyclosporin A, Witconol RHP®, and Tea tree oil.

Another embodiment comprises cyclosporin A, a Lanolin-based Derivative, and Tea tree oil.

Another embodiment comprises cyclosporin A, Amerchol CAB®, and Tea tree oil.

Another embodiment comprises cyclosporin A, Amerchol L-101®, and Tea tree oil.

Another embodiment comprises cyclosporin A, Amerlate LFA-LO®, and Tea tree oil.

Another embodiment comprises cyclosporin A, Amerlate P®, and Tea tree oil.

Another embodiment comprises cyclosporin A, Barre Common Degras®, and Tea tree oil.

Another embodiment comprises cyclosporin A, Cholesterol NF®, and Tea tree oil.

Another embodiment comprises cyclosporin A, Crodalan AWS®, and Tea tree oil.

Another embodiment comprises cyclosporin A, Crodalan LA®, and Tea tree oil.

Another embodiment comprises cyclosporin A, Emery 1650®, and Tea tree oil.

Another embodiment comprises cyclosporin A, Emery 1650®, and Tea tree oil.

Another embodiment comprises cyclosporin A, Emery 1740®, and Tea tree oil.

Another embodiment comprises cyclosporin A, Emery 1740®, and Tea tree oil.

Another embodiment comprises cyclosporin A, Forlan 500®, and Tea tree oil.

Another embodiment comprises cyclosporin A, Forlan L®, and Tea tree oil.

Another embodiment comprises cyclosporin A, Laneto 50®, and Tea tree oil.

Another embodiment comprises cyclosporin A, Laneto 100®, and Tea tree oil.

Another embodiment comprises cyclosporin A, Laneto AWS®, and Tea tree oil.

Another embodiment comprises cyclosporin A, Lanfrax 1776®, and Tea tree oil.

Another embodiment comprises cyclosporin A, Lanfrax 1776®, and Tea tree oil.

Another embodiment comprises cyclosporin A, Lanogel 21®, and Tea tree oil.

Another embodiment comprises cyclosporin A, Lipolan®, and Tea tree oil.

Another embodiment comprises cyclosporin A, Lipolan 31®, and Tea tree oil.

Another embodiment comprises cyclosporin A, OHlan®, and Tea tree oil.

Another embodiment comprises cyclosporin A, Polychol 5®, and Tea tree oil.

Another embodiment comprises cyclosporin A, Polychol 15®, and Tea tree oil.

Another embodiment comprises cyclosporin A, Ritacetyl®, and Tea tree oil.

Another embodiment comprises cyclosporin A, Ritachol®, and Tea tree oil.

Another embodiment comprises cyclosporin A, Ritahydrox®, and Tea tree oil.

Another embodiment comprises cyclosporin A, Ritalafa®, and Tea tree oil.

Another embodiment comprises cyclosporin A, Ritalan®, and Tea tree oil.

Another embodiment comprises cyclosporin A, Ritalan AWS®, and Tea tree oil.

Another embodiment comprises cyclosporin A, Ritalan C®, and Tea tree oil.

Another embodiment comprises cyclosporin A, Ritawax®, and Tea tree oil.

Another embodiment comprises cyclosporin A, Ritawax AEO®, and Tea tree oil.

Another embodiment comprises cyclosporin A, Ritawax ALA®, and Tea tree oil.

Another embodiment comprises cyclosporin A, Solan/Solan 50/Super Solan®, and Tea tree oil.

Another embodiment comprises cyclosporin A, Super Hartolan/Hartolan®, and Tea tree oil.

Another embodiment comprises cyclosporin A, Supersat AWS-4®, and Tea tree oil.

Another embodiment comprises cyclosporin A, Supersat AWS-24®, and Tea tree oil.

Another embodiment comprises cyclosporin A, a Lecithin or Lecithin Derivative, and Tea tree oil.

Another embodiment comprises cyclosporin A, Alcolec®, and Tea tree oil.

Another embodiment comprises cyclosporin A, Lecithin®, and Tea tree oil.

Another embodiment comprises cyclosporin A, Lexin K®, and Tea tree oil.

Another embodiment comprises cyclosporin A, Natipide®, and Tea tree oil.

Another embodiment comprises cyclosporin A, a Lignin or Lignin Derivative, and Tea tree oil.

Another embodiment comprises cyclosporin A, Diwatex XP 9®, and Tea tree oil.

Another embodiment comprises cyclosporin A, Dynasperse LCD®, and Tea tree oil.

Another embodiment comprises cyclosporin A, Indulin SAL®, and Tea tree oil.

Another embodiment comprises cyclosporin A, Indulin W-1®, and Tea tree oil.

Another embodiment comprises cyclosporin A, Indulin W-5®, and Tea tree oil.

Another embodiment comprises cyclosporin A, Lignosol FTA®, and Tea tree oil.

Another embodiment comprises cyclosporin A, Lignosol SFX-65®, and Tea tree oil.

Another embodiment comprises cyclosporin A, Marasperse 52 CP®, and Tea tree oil.

Another embodiment comprises cyclosporin A, Marasperse AG®, and Tea tree oil.

Another embodiment comprises cyclosporin A, Marasperse CBOS-4®, and Tea tree oil.

Another embodiment comprises cyclosporin A, Ufoxane 2®, and Tea tree oil.

Another embodiment comprises cyclosporin A, a Methyl Ester, and Tea tree oil.

Another embodiment comprises cyclosporin A, E.B. Cleaner AK®, and Tea tree oil.

Another embodiment comprises cyclosporin A, Oleocal ME-70®, and Tea tree oil.

Another embodiment comprises cyclosporin A, Trade Name Company®, and Tea tree oil.

Another embodiment comprises cyclosporin A, Methyl Esters (cont'd)®, and Tea tree oil.

Another embodiment comprises cyclosporin A, Oleocal ME-92®, and Tea tree oil.

Another embodiment comprises cyclosporin A, Oleocal ME-112®, and Tea tree oil.

Another embodiment comprises cyclosporin A, Oleocal ME-130®, and Tea tree oil.

Another embodiment comprises cyclosporin A, a Monoglyceride or a Derivative thereof, and Tea tree oil.

Another embodiment comprises cyclosporin A, Dynacet 211®, and Tea tree oil.

Another embodiment comprises cyclosporin A, Hetsorb S-20®, and Tea tree oil.

Another embodiment comprises cyclosporin A, Imwitor 191®, and Tea tree oil.

Another embodiment comprises cyclosporin A, Imwitor 370®, and Tea tree oil.

Another embodiment comprises cyclosporin A, Imwitor 375®, and Tea tree oil.

Another embodiment comprises cyclosporin A, Imwitor 900®, and Tea tree oil.

Another embodiment comprises cyclosporin A, Imwitor 945®, and Tea tree oil.

Another embodiment comprises cyclosporin A, Imwitor 2020®, and Tea tree oil.

Another embodiment comprises cyclosporin A, Kemester 5500®, and Tea tree oil.

Another embodiment comprises cyclosporin A, Kemester 6000®, and Tea tree oil.

Another embodiment comprises cyclosporin A, Magrabar GMC®, and Tea tree oil.

Another embodiment comprises cyclosporin A, Magrabar GMO-CK®, and Tea tree oil.

Another embodiment comprises cyclosporin A, Magrabar GPC-10®, and Tea tree oil.

Another embodiment comprises cyclosporin A, Magrabar MDG-5050®, and Tea tree oil.

Another embodiment comprises cyclosporin A, Monalube 335®, and Tea tree oil.

Another embodiment comprises cyclosporin A, Monoglycerides Glyceryl Monestearate Archer Daniels Midland Company®, and Tea tree oil.

Another embodiment comprises cyclosporin A, Rita GMS®, and Tea tree oil.

Another embodiment comprises cyclosporin A, Ritamulse SCG®, and Tea tree oil.

Another embodiment comprises cyclosporin A, Softigen 701®, and Tea tree oil.

Another embodiment comprises cyclosporin A, Tally 100 Plus®, and Tea tree oil.

Another embodiment comprises cyclosporin A, a Polyethylene Glycol, and Tea tree oil.

Another embodiment comprises cyclosporin A, Emulgade PL 68/50®, and Tea tree oil.

Another embodiment comprises cyclosporin A, Lumulse PEG®, and Tea tree oil.

Another embodiment comprises cyclosporin A, Rhodasurf PEG-400®, and Tea tree oil.

Another embodiment comprises cyclosporin A, Rhodasurf PEG-600®, and Tea tree oil.

Another embodiment comprises cyclosporin A, Witconol PEG-400®, and Tea tree oil.

Another embodiment comprises cyclosporin A, a Polymeric Surfactant, and Tea tree oil.

Another embodiment comprises cyclosporin A, Acritamer PNC-EG®, and Tea tree oil.

Another embodiment comprises cyclosporin A, Ag-Rho DEP-775®, and Tea tree oil.

Another embodiment comprises cyclosporin A, APG 325N Glycosidee, and Tea tree oil.

Another embodiment comprises cyclosporin A, Aristoflex AVC®, and Tea tree oil.

Another embodiment comprises cyclosporin A, Aristoflex HMB®, and Tea tree oil.

Another embodiment comprises cyclosporin A, Burco NPS-225®, and Tea tree oil.

Another embodiment comprises cyclosporin A, Burco NPS-816®, and Tea tree oil.

Another embodiment comprises cyclosporin A, Chemccinate 5603®, and Tea tree oil.

Another embodiment comprises cyclosporin A, Cosmedia Guar C-261N®, and Tea tree oil.

Another embodiment comprises cyclosporin A, Gantrez S-95®, and Tea tree oil.

Another embodiment comprises cyclosporin A, Glucopon 220 UP®, and Tea tree oil.

Another embodiment comprises cyclosporin A, Glucopon 225 DK®, and Tea tree oil.

Another embodiment comprises cyclosporin A, Glucopon 425 N®, and Tea tree oil.

Another embodiment comprises cyclosporin A, Glucopon 600 UP®, and Tea tree oil.

Another embodiment comprises cyclosporin A, Glucopon 625 UP®, and Tea tree oil.

Another embodiment comprises cyclosporin A, Pemulen 1621®, and Tea tree oil.

Another embodiment comprises cyclosporin A, Pemulen 1622®, and Tea tree oil.

Another embodiment comprises cyclosporin A, Pemulen TR-1®, and Tea tree oil.

Another embodiment comprises cyclosporin A, Pemulen TR-2®, and Tea tree oil.

Another embodiment comprises cyclosporin A, Plantacare 818®, and Tea tree oil.

Another embodiment comprises cyclosporin A, Plantapon LGC Sorb®, and Tea tree oil.

Another embodiment comprises cyclosporin A, Plantaren 1200N®, and Tea tree oil.

Another embodiment comprises cyclosporin A, Plantaren 2000N®, and Tea tree oil.

Another embodiment comprises cyclosporin A, Viscolam AT 64®, and Tea tree oil.

Another embodiment comprises cyclosporin A, Viscolam AT 64P®, and Tea tree oil.

Another embodiment comprises cyclosporin A, Viscolam AT 100®, and Tea tree oil.

Another embodiment comprises cyclosporin A, Viscolam MAC 7®, and Tea tree oil.

Another embodiment comprises cyclosporin A, Viscolam SMC 20®, and Tea tree oil.

Another embodiment comprises cyclosporin A, Witbreak RTC-323®, and Tea tree oil.

Another embodiment comprises cyclosporin A, WSI 3700®, and Tea tree oil.

Another embodiment comprises cyclosporin A, a Propoxylated or Ethoxylated Fatty Acid, Alcohol, or Alkyl Phenol, and Tea tree oil.

Another embodiment comprises cyclosporin A, Antarox AA-60®, and Tea tree oil.

Another embodiment comprises cyclosporin A, Antarox LF-224®, and Tea tree oil.

Another embodiment comprises cyclosporin A, Burcomul DFE-45®, and Tea tree oil.

Another embodiment comprises cyclosporin A, Burcoterge LFE-1000®, and Tea tree oil.

Another embodiment comprises cyclosporin A, Chemal LF-25B®, and Tea tree oil.

Another embodiment comprises cyclosporin A, Chemal LF-40B®, and Tea tree oil.

Another embodiment comprises cyclosporin A, Dehypon LS-36®, and Tea tree oil.

Another embodiment comprises cyclosporin A, Dehypon LS-36®, and Tea tree oil.

Another embodiment comprises cyclosporin A, Dehypon LS-54®, and Tea tree oil.

Another embodiment comprises cyclosporin A, Dehypon LS-54®, and Tea tree oil.

Another embodiment comprises cyclosporin A, Delonic 100 VLF®, and Tea tree oil.

Another embodiment comprises cyclosporin A, Delonic LF-60 MOD®, and Tea tree oil.

Another embodiment comprises cyclosporin A, Empiderm B®, and Tea tree oil.

Another embodiment comprises cyclosporin A, Ethylan 1206®, and Tea tree oil.

Another embodiment comprises cyclosporin A, Ethylan NS-500K®, and Tea tree oil.

Another embodiment comprises cyclosporin A, Ethylan NS-500LQ®, and Tea tree oil.

Another embodiment comprises cyclosporin A, Genapol 1392®, and Tea tree oil.

Another embodiment comprises cyclosporin A, Genapol 2317®, and Tea tree oil.

Another embodiment comprises cyclosporin A, Genapol 26EP710®, and Tea tree oil.

Another embodiment comprises cyclosporin A, Genapol EP 1022®, and Tea tree oil.

Another embodiment comprises cyclosporin A, Genapol EP 1024®, and Tea tree oil.

Another embodiment comprises cyclosporin A, Genapol EP 6068®, and Tea tree oil.

Another embodiment comprises cyclosporin A, Genapol NP915®, and Tea tree oil.

Another embodiment comprises cyclosporin A, Kieralon MFB®, and Tea tree oil.

Another embodiment comprises cyclosporin A, Lumisolve CSA-80 V®, and Tea tree oil.

Another embodiment comprises cyclosporin A, Marlowet 5001®, and Tea tree oil.

Another embodiment comprises cyclosporin A, Marlox FK 64®, and Tea tree oil.

Another embodiment comprises cyclosporin A, Marlox MO 124®, and Tea tree oil.

Another embodiment comprises cyclosporin A, Marlox S 58®, and Tea tree oil.

Another embodiment comprises cyclosporin A, Nonatell 1003®, and Tea tree oil.

Another embodiment comprises cyclosporin A, Nonatell 1038®, and Tea tree oil.

Another embodiment comprises cyclosporin A, Nonatell 1052®, and Tea tree oil.

Another embodiment comprises cyclosporin A, Nonatell 1061®, and Tea tree oil.

Another embodiment comprises cyclosporin A, Nonatell 1075®, and Tea tree oil.

Another embodiment comprises cyclosporin A, Nonatell 1088®, and Tea tree oil.

Another embodiment comprises cyclosporin A, Nonatell 1123®, and Tea tree oil.

Another embodiment comprises cyclosporin A, Nonatell 1153®, and Tea tree oil.

Another embodiment comprises cyclosporin A, Nonatell 1161®, and Tea tree oil.

Another embodiment comprises cyclosporin A, Nonatell 1172®, and Tea tree oil.

Another embodiment comprises cyclosporin A, Nonatell 1181®, and Tea tree oil.

Another embodiment comprises cyclosporin A, Norfox 36®, and Tea tree oil.

Another embodiment comprises cyclosporin A, Procetyl AWS®, and Tea tree oil.

Another embodiment comprises cyclosporin A, Sandoxylate SX 412®, and Tea tree oil.

Another embodiment comprises cyclosporin A, Sandoxylate SX 418®, and Tea tree oil.

Another embodiment comprises cyclosporin A, Surfonic JL-80X®, and Tea tree oil.

Another embodiment comprises cyclosporin A, Surfonic JL-80X-B1®, and Tea tree oil.

Another embodiment comprises cyclosporin A, Surfonic L4-29X®, and Tea tree oil.

Another embodiment comprises cyclosporin A, Surfonic LF®, and Tea tree oil.

Another embodiment comprises cyclosporin A, T-Det A826®, and Tea tree oil.

Another embodiment comprises cyclosporin A, T-Det LF-416®, and Tea tree oil.

Another embodiment comprises cyclosporin A, Tergitol Min-Foam 1X®, and Tea tree oil.

Another embodiment comprises cyclosporin A, Tergitol Min-Foam 2X®, and Tea tree oil.

Another embodiment comprises cyclosporin A, Triton CF-21®, and Tea tree oil.

Another embodiment comprises cyclosporin A, Triton CF-76®, and Tea tree oil.

Another embodiment comprises cyclosporin A, Triton XL-80N®, and Tea tree oil.

Another embodiment comprises cyclosporin A, Witconol NS-98®, and Tea tree oil.

Another embodiment comprises cyclosporin A, Witconol NS-108LQ®, and Tea tree oil.

Another embodiment comprises cyclosporin A, Witconol NS-145®, and Tea tree oil.

Another embodiment comprises cyclosporin A, Witconol NS-179®, and Tea tree oil.

Another embodiment comprises cyclosporin A, a Protein-based Surfactant, and Tea tree oil.

Another embodiment comprises cyclosporin A, AminoFoam W®, and Tea tree oil.

Another embodiment comprises cyclosporin A, Amiter LGOD-2®, and Tea tree oil.

Another embodiment comprises cyclosporin A, Amiter LGS-2®, and Tea tree oil.

Another embodiment comprises cyclosporin A, Amiter LGS-5®, and Tea tree oil.

Another embodiment comprises cyclosporin A, Lamepon S®, and Tea tree oil.

Another embodiment comprises cyclosporin A, Lamepon S®, and Tea tree oil.

Another embodiment comprises cyclosporin A, Maypon 4C®, and Tea tree oil.

Another embodiment comprises cyclosporin A, May-Tein C®, and Tea tree oil.

Another embodiment comprises cyclosporin A, May-Tein CT®, and Tea tree oil.

Another embodiment comprises cyclosporin A, May-Tein KTS®, and Tea tree oil.

Another embodiment comprises cyclosporin A, May-Tein SY®, and Tea tree oil.

Another embodiment comprises cyclosporin A, Plantapon S®, and Tea tree oil.

Another embodiment comprises cyclosporin A, Proteol APL®, and Tea tree oil.

Another embodiment comprises cyclosporin A, Proteol OAT®, and Tea tree oil.

Another embodiment comprises cyclosporin A, Pyroter CPI-40®, and Tea tree oil.

Another embodiment comprises cyclosporin A, Pyroter GPI-25®, and Tea tree oil.

Another embodiment comprises cyclosporin A, Supro-Tein S®, and Tea tree oil.

Another embodiment comprises cyclosporin A, Supro-Tein V®, and Tea tree oil.

Another embodiment comprises cyclosporin A,® and Tea tree oil.

Another embodiment comprises cyclosporin A, a Sarcosine Derivative®, and Tea tree oil.

Another embodiment comprises cyclosporin A, Crodasinic LS-30®, and Tea tree oil.

Another embodiment comprises cyclosporin A, Vanseal CS®, and Tea tree oil.

Another embodiment comprises cyclosporin A, Vanseal LS®, and Tea tree oil.

Another embodiment comprises cyclosporin A, Vanseal MS®, and Tea tree oil.

Another embodiment comprises cyclosporin A, Vanseal NACS-30®, and Tea tree oil.

Another embodiment comprises cyclosporin A, Vanseal NALS-95®, and Tea tree oil.

Another embodiment comprises cyclosporin A, Vanseal OS®, and Tea tree oil.

Another embodiment comprises cyclosporin A,® and Tea tree oil.

Another embodiment comprises cyclosporin A, a Silicone-based Surfactant®, and Tea tree oil.

Another embodiment comprises cyclosporin A, Abil-B-9950®, and Tea tree oil.

Another embodiment comprises cyclosporin A, Abil Care 85®, and Tea tree oil.

Another embodiment comprises cyclosporin A, Abil EM 90®, and Tea tree oil.

Another embodiment comprises cyclosporin A, Abil EM 97®, and Tea tree oil.

Another embodiment comprises cyclosporin A, Abil WE-09®, and Tea tree oil.

Another embodiment comprises cyclosporin A, Dow Corning 1248 Fluid®, and Tea tree oil.

Another embodiment comprises cyclosporin A, Dow Corning 3225C®, and Tea tree oil.

Another embodiment comprises cyclosporin A, Dow Corning 5200®, and Tea tree oil.

Another embodiment comprises cyclosporin A, Dow Corning Q4-3667®, and Tea tree oil.

Another embodiment comprises cyclosporin A, Monasil PCA®, and Tea tree oil.

Another embodiment comprises cyclosporin A, Monasil PDM®, and Tea tree oil.

Another embodiment comprises cyclosporin A, Monasil PLN®, and Tea tree oil.

Another embodiment comprises cyclosporin A, Polyderm PPI-SI-WS®, and Tea tree oil.

Another embodiment comprises cyclosporin A, Troysol 380W®, and Tea tree oil.

Another embodiment comprises cyclosporin A, Troysol S366®, and Tea tree oil.

Another embodiment comprises cyclosporin A, a Sorbitan Derivative, and Tea tree oil.

Another embodiment comprises cyclosporin A, Alkamuls SML®, and Tea tree oil.

Another embodiment comprises cyclosporin A, Alkamuls SMO®, and Tea tree oil.

Another embodiment comprises cyclosporin A, Alkamuls STO®, and Tea tree oil.

Another embodiment comprises cyclosporin A, Arlacel 20®, and Tea tree oil.

Another embodiment comprises cyclosporin A, Arlacel 40®, and Tea tree oil.

Another embodiment comprises cyclosporin A, Arlacel 60®, and Tea tree oil.

Another embodiment comprises cyclosporin A, Arlacel 80®, and Tea tree oil.

Another embodiment comprises cyclosporin A, Arlacel C®, and Tea tree oil.

Another embodiment comprises cyclosporin A, Armul 21®, and Tea tree oil.

Another embodiment comprises cyclosporin A, Atlox 80®, and Tea tree oil.

Another embodiment comprises cyclosporin A, Atlox 847®, and Tea tree oil.

Another embodiment comprises cyclosporin A, Atlox 1045A®, and Tea tree oil.

Another embodiment comprises cyclosporin A, Canarcel 20®, and Tea tree oil.

Another embodiment comprises cyclosporin A, Canarcel 60®, and Tea tree oil.

Another embodiment comprises cyclosporin A, Canarcel 80®, and Tea tree oil.

Another embodiment comprises cyclosporin A, Canarcel TW 20®, and Tea tree oil.

Another embodiment comprises cyclosporin A, Canarcel TW 60®, and Tea tree oil.

Another embodiment comprises cyclosporin A, Canarcel TW 80®, and Tea tree oil.

Another embodiment comprises cyclosporin A, Coladet BSB-P®, and Tea tree oil.

Another embodiment comprises cyclosporin A, Customulse O-20®, and Tea tree oil.

Another embodiment comprises cyclosporin A, Dehymuls E®, and Tea tree oil.

Another embodiment comprises cyclosporin A, DeSotan SMO®, and Tea tree oil.

Another embodiment comprises cyclosporin A, DeSotan SMO-20®, and Tea tree oil.

Another embodiment comprises cyclosporin A, DeSotan SMT®, and Tea tree oil.

Another embodiment comprises cyclosporin A, DeSotan SMT-20®, and Tea tree oil.

Another embodiment comprises cyclosporin A, Durfax 60®, and Tea tree oil.

Another embodiment comprises cyclosporin A, Durfax 65®, and Tea tree oil.

Another embodiment comprises cyclosporin A, Durfax 80®, and Tea tree oil.

Another embodiment comprises cyclosporin A, Durtan 60®, and Tea tree oil.

Another embodiment comprises cyclosporin A, Durtan 65®, and Tea tree oil.

Another embodiment comprises cyclosporin A, Liposorb L®, and Tea tree oil.

Another embodiment comprises cyclosporin A, Liposorb L-10®, and Tea tree oil.

Another embodiment comprises cyclosporin A, Liposorb L-20®, and Tea tree oil.

Another embodiment comprises cyclosporin A, Liposorb O®, and Tea tree oil.

Another embodiment comprises cyclosporin A, Liposorb O-20®, and Tea tree oil.

Another embodiment comprises cyclosporin A, Liposorb P®, and Tea tree oil.

Another embodiment comprises cyclosporin A, Liposorb P-20®, and Tea tree oil.

Another embodiment comprises cyclosporin A, Liposorb S®, and Tea tree oil.

Another embodiment comprises cyclosporin A, Liposorb S-20®, and Tea tree oil.

Another embodiment comprises cyclosporin A, Liposorb SQO®, and Tea tree oil.

Another embodiment comprises cyclosporin A, Liposorb TO®, and Tea tree oil.

Another embodiment comprises cyclosporin A, Liposorb TS®, and Tea tree oil.

Another embodiment comprises cyclosporin A, Liposorb TS-20®, and Tea tree oil.

Another embodiment comprises cyclosporin A, Lumisorb PS®, and Tea tree oil.

Another embodiment comprises cyclosporin A, Lumisorb SMO (T)®, and Tea tree oil.

Another embodiment comprises cyclosporin A, Lumisorb SMS K®, and Tea tree oil.

Another embodiment comprises cyclosporin A, Lumisorb SSO®, and Tea tree oil.

Another embodiment comprises cyclosporin A, Lumisorb STS K®, and Tea tree oil.

Another embodiment comprises cyclosporin A, Lumisorb STT®, and Tea tree oil.

Another embodiment comprises cyclosporin A, Magrabar SMO®, and Tea tree oil.

Another embodiment comprises cyclosporin A, Magrabar SMO-VEG®, and Tea tree oil.

Another embodiment comprises cyclosporin A, Magrabar SMT®, and Tea tree oil.

Another embodiment comprises cyclosporin A, Magrabar STO®, and Tea tree oil.

Another embodiment comprises cyclosporin A, Miracare BC-27®, and Tea tree oil.

Another embodiment comprises cyclosporin A, Ritabate 20®, and Tea tree oil.

Another embodiment comprises cyclosporin A, Ritabate 40®, and Tea tree oil.

Another embodiment comprises cyclosporin A, Ritabate 60®, and Tea tree oil.

Another embodiment comprises cyclosporin A, Ritabate 80®, and Tea tree oil.

Another embodiment comprises cyclosporin A, T-Maz®, and Tea tree oil.

Another embodiment comprises cyclosporin A, Tego SML®, and Tea tree oil.

Another embodiment comprises cyclosporin A, Tego SML 20®, and Tea tree oil.

Another embodiment comprises cyclosporin A, Tego SMO 80 V®, and Tea tree oil.

Another embodiment comprises cyclosporin A, Tego SMO V®, and Tea tree oil.

Another embodiment comprises cyclosporin A, Tego SMS®, and Tea tree oil.

Another embodiment comprises cyclosporin A, Tego STO V®, and Tea tree oil.

Another embodiment comprises cyclosporin A, Tween 21®, and Tea tree oil.

Another embodiment comprises cyclosporin A, Tween 40®, and Tea tree oil.

Another embodiment comprises cyclosporin A, Tween 60®, and Tea tree oil.

Another embodiment comprises cyclosporin A, Tween 60 K®, and Tea tree oil.

Another embodiment comprises cyclosporin A, Tween 61®, and Tea tree oil.

Another embodiment comprises cyclosporin A, Tween 65®, and Tea tree oil.

Another embodiment comprises cyclosporin A, Tween 80®, and Tea tree oil.

Another embodiment comprises cyclosporin A, Tween 80 K®, and Tea tree oil.

Another embodiment comprises cyclosporin A, Tween 81®, and Tea tree oil.

Another embodiment comprises cyclosporin A, Tween 85®, and Tea tree oil.

Another embodiment comprises cyclosporin A, a Sucrose or Glucose Ester, or Derivative thereof, and Tea tree oil.

Another embodiment comprises cyclosporin A, DeSulf GOS-P-60WCG®, and Tea tree oil.

Another embodiment comprises cyclosporin A, Glucam E-20 Distearate®, and Tea tree oil.

Another embodiment comprises cyclosporin A, Glucamate DOE-120®, and Tea tree oil.

Another embodiment comprises cyclosporin A, Glucamate SSE-20®, and Tea tree oil.

Another embodiment comprises cyclosporin A, Glucate DO®, and Tea tree oil.

Another embodiment comprises cyclosporin A, Glucate SS®, and Tea tree oil.

Another embodiment comprises cyclosporin A, Glucopon 425 UP®, and Tea tree oil.

Another embodiment comprises cyclosporin A, Isolan IS®, and Tea tree oil.

Another embodiment comprises cyclosporin A, Mazon 40®, and Tea tree oil.

Another embodiment comprises cyclosporin A, Montanov 82®, and Tea tree oil.

Another embodiment comprises cyclosporin A, Montanov 202®, and Tea tree oil.

Another embodiment comprises cyclosporin A, Montanov S®, and Tea tree oil.

Another embodiment comprises cyclosporin A, Rheozan®, and Tea tree oil.

Another embodiment comprises cyclosporin A, Simulsol AS 48®, and Tea tree oil.

Another embodiment comprises cyclosporin A, Simulsol SL 4®, and Tea tree oil.

Another embodiment comprises cyclosporin A, Simulsol SL 10®, and Tea tree oil.

Another embodiment comprises cyclosporin A, Simulsol SL 11W®, and Tea tree oil.

Another embodiment comprises cyclosporin A, Simulsol SL 55®, and Tea tree oil.

Another embodiment comprises cyclosporin A, Suga Nate 100 and 160®, and Tea tree oil.

Another embodiment comprises cyclosporin A, Tego Care 450®, and Tea tree oil.

Another embodiment comprises cyclosporin A, Tego Care CG 90®, and Tea tree oil.

Another embodiment comprises cyclosporin A, Tego Care PS®, and Tea tree oil.

Another embodiment comprises cyclosporin A, Tegosoft PSE 141 G®, and Tea tree oil.

Another embodiment comprises cyclosporin A, Tegotens G 826®, and Tea tree oil.

Another embodiment comprises cyclosporin A, Triton BG-10 (70%)®, and Tea tree oil.

Another embodiment comprises cyclosporin A, Triton CG-110 (60%)®, and Tea tree oil.

Another embodiment comprises cyclosporin A, Wickenol 545®, and Tea tree oil.

Another embodiment comprises cyclosporin A, an oil having a specific gravity from 0.95 to 1.07, and pluronic.

Another embodiment comprises cyclosporin A, Anise oil, and pluronic.

Another embodiment comprises cyclosporin A, Castor oil, and pluronic.

Another embodiment comprises cyclosporin A, Clove oil, and pluronic.

Another embodiment comprises cyclosporin A, Cassia oil, and pluronic.

Another embodiment comprises cyclosporin A, Cinnamon oil, and pluronic.

Another embodiment comprises cyclosporin A, an oil having a Specific Gravity between 0.90 and 0.95, and pluronic.

Another embodiment comprises cyclosporin A, Almond oil, and pluronic.

Another embodiment comprises cyclosporin A, Corn oil, and pluronic.

Another embodiment comprises cyclosporin A, Arachis oil, and pluronic.

Another embodiment comprises cyclosporin A, Cottonseed oil, and pluronic.

Another embodiment comprises cyclosporin A, Safflower oil, and pluronic.

Another embodiment comprises cyclosporin A, Maize oil, and pluronic.

Another embodiment comprises cyclosporin A, Linseed oil, and pluronic.

Another embodiment comprises cyclosporin A, Rapeseed oil, and pluronic.

Another embodiment comprises cyclosporin A, Soybean oil, and pluronic.

Another embodiment comprises cyclosporin A, Olive oil, and pluronic.

Another embodiment comprises cyclosporin A, Caraway oil, and pluronic.

Another embodiment comprises cyclosporin A, Rosemary oil, and pluronic.

Another embodiment comprises cyclosporin A, Peanut oil, and pluronic.

Another embodiment comprises cyclosporin A, Peppermint oil, and pluronic.

Another embodiment comprises cyclosporin A, Sunflower oil, and pluronic.

Another embodiment comprises cyclosporin A, Eucalyptus oil, and pluronic.

Another embodiment comprises cyclosporin A, Sesame oil, and pluronic.

Another embodiment comprises cyclosporin A, an oil having a Specific Gravity below 0.9, and pluronic.

Another embodiment comprises cyclosporin A, Mineral oil, and pluronic.

Another embodiment comprises cyclosporin A, Coriander oil, and pluronic.

Another embodiment comprises cyclosporin A, Lavender oil, and pluronic.

Another embodiment comprises cyclosporin A, Citronella oil, and pluronic.

Another embodiment comprises cyclosporin A, Juniper oil, and pluronic.

Another embodiment comprises cyclosporin A, Lemon oil, and pluronic.

Another embodiment comprises cyclosporin A, Orange oil, and pluronic.

Another embodiment comprises cyclosporin A, Clary sage oil, and pluronic.

Another embodiment comprises cyclosporin A, Nutmeg oil, and pluronic.

Another embodiment comprises cyclosporin A, Tea tree oil, and pluronic.

Another embodiment comprises cyclosporin A, Abex 2515®, and an oil having a specific gravity from 0.95 to 1.07.

Another embodiment comprises cyclosporin A, Abex 2525/40®, and an oil having a specific gravity from 0.95 to 1.07.

Another embodiment comprises cyclosporin A, Abex 2535®, and an oil having a specific gravity from 0.95 to 1.07.

Another embodiment comprises cyclosporin A, Abex 2545®, and an oil having a specific gravity from 0.95 to 1.07.

Another embodiment comprises cyclosporin A, Acconon E®, and an oil having a specific gravity from 0.95 to 1.07.

Another embodiment comprises cyclosporin A, Alfonic 1216CO-9®, and an oil having a specific gravity from 0.95 to 1.07.

Another embodiment comprises cyclosporin A, Alfonic 1412-3®, and an oil having a specific gravity from 0.95 to 1.07.

Another embodiment comprises cyclosporin A, Alfonic TDA-12®, and an oil having a specific gravity from 0.95 to 1.07.

Another embodiment comprises cyclosporin A, Alfonic TDA-3®, and an oil having a specific gravity from 0.95 to 1.07.

Another embodiment comprises cyclosporin A, Alfonic TDA-4®, and an oil having a specific gravity from 0.95 to 1.07.

Another embodiment comprises cyclosporin A, Alfonic TDA-6®, and an oil having a specific gravity from 0.95 to 1.07.

Another embodiment comprises cyclosporin A, Alfonic TDA-7®, and an oil having a specific gravity from 0.95 to 1.07.

Another embodiment comprises cyclosporin A, Alfonic TDA-8®, and an oil having a specific gravity from 0.95 to 1.07.

Another embodiment comprises cyclosporin A, Alfonic TDA-9®, and an oil having a specific gravity from 0.95 to 1.07.

Another embodiment comprises cyclosporin A, Alkamuls 400-DO®, and an oil having a specific gravity from 0.95 to 1.07.

Another embodiment comprises cyclosporin A, Alkamuls 400-MO/E®, and an oil having a specific gravity from 0.95 to 1.07.

Another embodiment comprises cyclosporin A, Ameroxol OE-10®, and an oil having a specific gravity from 0.95 to 1.07.

Another embodiment comprises cyclosporin A, Ameroxol OE-2®, and an oil having a specific gravity from 0.95 to 1.07.

Another embodiment comprises cyclosporin A, Ameroxol OE-20®, and an oil having a specific gravity from 0.95 to 1.07.

Another embodiment comprises cyclosporin A, Armix 176®, and an oil having a specific gravity from 0.95 to 1.07.

Another embodiment comprises cyclosporin A, Armix 180-C®, and an oil having a specific gravity from 0.95 to 1.07.

Another embodiment comprises cyclosporin A, Armix 183®, and an oil having a specific gravity from 0.95 to 1.07.

Another embodiment comprises cyclosporin A, Brij 35®, and an oil having a specific gravity from 0.95 to 1.07.

Another embodiment comprises cyclosporin A, Brij 35 Liquid®, and an oil having a specific gravity from 0.95 to 1.07.

Another embodiment comprises cyclosporin A, Brij 35 SP®, and an oil having a specific gravity from 0.95 to 1.07.

Another embodiment comprises cyclosporin A, Canasol MJ 2109®, and an oil having a specific gravity from 0.95 to 1.07.

Another embodiment comprises cyclosporin A, Canasol MJ 52®, and an oil having a specific gravity from 0.95 to 1.07.

Another embodiment comprises cyclosporin A, Canasol R 3600®, and an oil having a specific gravity from 0.95 to 1.07.

Another embodiment comprises cyclosporin A, Canasol R 3603®, and an oil having a specific gravity from 0.95 to 1.07.

Another embodiment comprises cyclosporin A, Canasol R 4000 H®, and an oil having a specific gravity from 0.95 to 1.07.

Another embodiment comprises cyclosporin A, Cedepal CA-210®, and an oil having a specific gravity from 0.95 to 1.07.

Another embodiment comprises cyclosporin A, Cedepal CA-520®, and an oil having a specific gravity from 0.95 to 1.07.

Another embodiment comprises cyclosporin A, Cedepal CA-630®, and an oil having a specific gravity from 0.95 to 1.07.

Another embodiment comprises cyclosporin A, Cedepal CA-720®, and an oil having a specific gravity from 0.95 to 1.07.

Another embodiment comprises cyclosporin A, Cedepal CA-890®, and an oil having a specific gravity from 0.95 to 1.07.

Another embodiment comprises cyclosporin A, Cedepal CA-897®, and an oil having a specific gravity from 0.95 to 1.07.

Another embodiment comprises cyclosporin A, Cedepal CO-210®, and an oil having a specific gravity from 0.95 to 1.07.

Another embodiment comprises cyclosporin A, Cedepal CO-430®, and an oil having a specific gravity from 0.95 to 1.07.

Another embodiment comprises cyclosporin A, Cedepal CO-530®, and an oil having a specific gravity from 0.95 to 1.07.

Another embodiment comprises cyclosporin A, Cedepal CO-630®, and an oil having a specific gravity from 0.95 to 1.07.

Another embodiment comprises cyclosporin A, Cedepal CO-710®, and an oil having a specific gravity from 0.95 to 1.07.

Another embodiment comprises cyclosporin A, Cedepal CO-730®, and an oil having a specific gravity from 0.95 to 1.07.

Another embodiment comprises cyclosporin A, Cedepal CO-880®, and an oil having a specific gravity from 0.95 to 1.07.

Another embodiment comprises cyclosporin A, Cedepal CO-887®, and an oil having a specific gravity from 0.95 to 1.07.

Another embodiment comprises cyclosporin A, Cedepal CO-890®, and an oil having a specific gravity from 0.95 to 1.07.

Another embodiment comprises cyclosporin A, Cedepal CO-897®, and an oil having a specific gravity from 0.95 to 1.07.

Another embodiment comprises cyclosporin A, Cedepal CO-970®, and an oil having a specific gravity from 0.95 to 1.07.

Another embodiment comprises cyclosporin A, Cedepal CO-977®, and an oil having a specific gravity from 0.95 to 1.07.

Another embodiment comprises cyclosporin A, Cedepal CO-990®, and an oil having a specific gravity from 0.95 to 1.07.

Another embodiment comprises cyclosporin A, Cedepal CO-997®, and an oil having a specific gravity from 0.95 to 1.07.

Another embodiment comprises cyclosporin A, Chemal BP 261®, and an oil having a specific gravity from 0.95 to 1.07.

Another embodiment comprises cyclosporin A, Chemal BP 262®, and an oil having a specific gravity from 0.95 to 1.07.

Another embodiment comprises cyclosporin A, Chemal BP 3172®, and an oil having a specific gravity from 0.95 to 1.07.

Another embodiment comprises cyclosporin A, Chemal BP 3174®, and an oil having a specific gravity from 0.95 to 1.07.

Another embodiment comprises cyclosporin A, Chemal DA-4®, and an oil having a specific gravity from 0.95 to 1.07.

Another embodiment comprises cyclosporin A, Chemal DA-6®, and an oil having a specific gravity from 0.95 to 1.07.

Another embodiment comprises cyclosporin A, Chemal DA-9®, and an oil having a specific gravity from 0.95 to 1.07.

Another embodiment comprises cyclosporin A, Chemal LA-23®, and an oil having a specific gravity from 0.95 to 1.07.

Another embodiment comprises cyclosporin A, Chemal LA-4®, and an oil having a specific gravity from 0.95 to 1.07.

Another embodiment comprises cyclosporin A, Chemal LA-9®, and an oil having a specific gravity from 0.95 to 1.07.

Another embodiment comprises cyclosporin A, Chemal OA-20G®, and an oil having a specific gravity from 0.95 to 1.07.

Another embodiment comprises cyclosporin A, Chemal OA-5®, and an oil having a specific gravity from 0.95 to 1.07.

Another embodiment comprises cyclosporin A, Chemal OA-9®, and an oil having a specific gravity from 0.95 to 1.07.

Another embodiment comprises cyclosporin A, Chemal TDA-12®, and an oil having a specific gravity from 0.95 to 1.07.

Another embodiment comprises cyclosporin A, Chemal TDA-15®, and an oil having a specific gravity from 0.95 to 1.07.

Another embodiment comprises cyclosporin A, Chemal TDA-3®, and an oil having a specific gravity from 0.95 to 1.07.

Another embodiment comprises cyclosporin A, Chemal TDA-6®, and an oil having a specific gravity from 0.95 to 1.07.

Another embodiment comprises cyclosporin A, Chemal TDA-9®, and an oil having a specific gravity from 0.95 to 1.07.

Another embodiment comprises cyclosporin A, Chemax CO-16®, and an oil having a specific gravity from 0.95 to 1.07.

Another embodiment comprises cyclosporin A, Chemax CO-200/50®, and an oil having a specific gravity from 0.95 to 1.07.

Another embodiment comprises cyclosporin A, Chemax CO-25®, and an oil having a specific gravity from 0.95 to 1.07.

Another embodiment comprises cyclosporin A, Chemax CO-30®, and an oil having a specific gravity from 0.95 to 1.07.

Another embodiment comprises cyclosporin A, Chemax CO-36®, and an oil having a specific gravity from 0.95 to 1.07.

Another embodiment comprises cyclosporin A, Chemax CO-40®, and an oil having a specific gravity from 0.95 to 1.07.

Another embodiment comprises cyclosporin A, Chemax CO-5®, and an oil having a specific gravity from 0.95 to 1.07.

Another embodiment comprises cyclosporin A, Chemax CO-80®, and an oil having a specific gravity from 0.95 to 1.07.

Another embodiment comprises cyclosporin A, Chemax DNP-150/50®, and an oil having a specific gravity from 0.95 to 1.07.

Another embodiment comprises cyclosporin A, Chemax DNP-18®, and an oil having a specific gravity from 0.95 to 1.07.

Another embodiment comprises cyclosporin A, Chemax DNP-8®, and an oil having a specific gravity from 0.95 to 1.07.

Another embodiment comprises cyclosporin A, Chemax E 200 ML®, and an oil having a specific gravity from 0.95 to 1.07.

Another embodiment comprises cyclosporin A, Chemax E 200 MO®, and an oil having a specific gravity from 0.95 to 1.07.

Another embodiment comprises cyclosporin A, Chemax E 400 ML®, and an oil having a specific gravity from 0.95 to 1.07.

Another embodiment comprises cyclosporin A, Chemax E 400 MO®, and an oil having a specific gravity from 0.95 to 1.07.

Another embodiment comprises cyclosporin A, Chemax E 600 ML®, and an oil having a specific gravity from 0.95 to 1.07.

Another embodiment comprises cyclosporin A, Chemax E 600 MO®, and an oil having a specific gravity from 0.95 to 1.07.

Another embodiment comprises cyclosporin A, Chemax E1000 MO®, and an oil having a specific gravity from 0.95 to 1.07.

Another embodiment comprises cyclosporin A, Chemax E1000 MS®, and an oil having a specific gravity from 0.95 to 1.07.

Another embodiment comprises cyclosporin A, Chemax E200 MS®, and an oil having a specific gravity from 0.95 to 1.07.

Another embodiment comprises cyclosporin A, Chemax E400 MS®, and an oil having a specific gravity from 0.95 to 1.07.

Another embodiment comprises cyclosporin A, Chemax E600 MS®, and an oil having a specific gravity from 0.95 to 1.07.

Another embodiment comprises cyclosporin A, Chemax HCO-16®, and an oil having a specific gravity from 0.95 to 1.07.

Another embodiment comprises cyclosporin A, Chemax HCO-200/50®, and an oil having a specific gravity from 0.95 to 1.07.

Another embodiment comprises cyclosporin A, Chemax HCO-25®, and an oil having a specific gravity from 0.95 to 1.07.

Another embodiment comprises cyclosporin A, Chemax HCO-5®, and an oil having a specific gravity from 0.95 to 1.07.

Another embodiment comprises cyclosporin A, Chemax TO-16®, and an oil having a specific gravity from 0.95 to 1.07.

Another embodiment comprises cyclosporin A, Chemax TO-8®, and an oil having a specific gravity from 0.95 to 1.07.

Another embodiment comprises cyclosporin A, Colalube 3409®, and an oil having a specific gravity from 0.95 to 1.07.

Another embodiment comprises cyclosporin A, Colalube 3411®, and an oil having a specific gravity from 0.95 to 1.07.

Another embodiment comprises cyclosporin A, Colalube 3413®, and an oil having a specific gravity from 0.95 to 1.07.

Another embodiment comprises cyclosporin A, Colalube 3414®, and an oil having a specific gravity from 0.95 to 1.07.

Another embodiment comprises cyclosporin A, Colalube 3415®, and an oil having a specific gravity from 0.95 to 1.07.

Another embodiment comprises cyclosporin A, Colalube 3416®, and an oil having a specific gravity from 0.95 to 1.07.

Another embodiment comprises cyclosporin A, Colalube 3417®, and an oil having a specific gravity from 0.95 to 1.07.

Another embodiment comprises cyclosporin A, Delonic NPE-100®, and an oil having a specific gravity from 0.95 to 1.07.

Another embodiment comprises cyclosporin A, Delonic NPE-30®, and an oil having a specific gravity from 0.95 to 1.07.

Another embodiment comprises cyclosporin A, Delonic NPE-40®, and an oil having a specific gravity from 0.95 to 1.07.

Another embodiment comprises cyclosporin A, Delonic OPE-10®, and an oil having a specific gravity from 0.95 to 1.07.

Another embodiment comprises cyclosporin A, Delonic OPE-12®, and an oil having a specific gravity from 0.95 to 1.07.

Another embodiment comprises cyclosporin A, Delonic OPE-30®, and an oil having a specific gravity from 0.95 to 1.07.

Another embodiment comprises cyclosporin A, Delonic OPE-40®, and an oil having a specific gravity from 0.95 to 1.07.

Another embodiment comprises cyclosporin A, Delonic OPE-5®, and an oil having a specific gravity from 0.95 to 1.07.

Another embodiment comprises cyclosporin A, Delonic OPE-7.5®, and an oil having a specific gravity from 0.95 to 1.07.

Another embodiment comprises cyclosporin A, DePeg 16-CO®, and an oil having a specific gravity from 0.95 to 1.07.

Another embodiment comprises cyclosporin A, DePeg 25-CO®, and an oil having a specific gravity from 0.95 to 1.07.

Another embodiment comprises cyclosporin A, DePeg 30-CO®, and an oil having a specific gravity from 0.95 to 1.07.

Another embodiment comprises cyclosporin A, DePeg 40-CO®, and an oil having a specific gravity from 0.95 to 1.07.

Another embodiment comprises cyclosporin A, DePeg 5-CO®, and an oil having a specific gravity from 0.95 to 1.07.

Another embodiment comprises cyclosporin A, DePeg 80-CO®, and an oil having a specific gravity from 0.95 to 1.07.

Another embodiment comprises cyclosporin A, DeSonic S-100, and an oil having a specific gravity from 0.95 to 1.07.

Another embodiment comprises cyclosporin A, DeSonic S-114®, and an oil having a specific gravity from 0.95 to 1.07.

Another embodiment comprises cyclosporin A, DeSonic S-405®, and an oil having a specific gravity from 0.95 to 1.07.

Another embodiment comprises cyclosporin A, DeSonic S-45®, and an oil having a specific gravity from 0.95 to 1.07.

Another embodiment comprises cyclosporin A, DeSonic TDA-9®, and an oil having a specific gravity from 0.95 to 1.07.

Another embodiment comprises cyclosporin A, DeThox Acid O-14®, and an oil having a specific gravity from 0.95 to 1.07.

Another embodiment comprises cyclosporin A, DeThox Acid O-16®, and an oil having a specific gravity from 0.95 to 1.07.

Another embodiment comprises cyclosporin A, DeThox Acid O-9®, and an oil having a specific gravity from 0.95 to 1.07.

Another embodiment comprises cyclosporin A, DeThox Acid TO-14®, and an oil having a specific gravity from 0.95 to 1.07.

Another embodiment comprises cyclosporin A, DeThox Acid TO-16.5®, and an oil having a specific gravity from 0.95 to 1.07.

Another embodiment comprises cyclosporin A, DeThox Acid TO-8.5®, and an oil having a specific gravity from 0.95 to 1.07.

Another embodiment comprises cyclosporin A, Dur-Em 114®, and an oil having a specific gravity from 0.95 to 1.07.

Another embodiment comprises cyclosporin A, Dur-Em 117®, and an oil having a specific gravity from 0.95 to 1.07.

Another embodiment comprises cyclosporin A, Dur-Em 204®, and an oil having a specific gravity from 0.95 to 1.07.

Another embodiment comprises cyclosporin A, Dur-Em 207®, and an oil having a specific gravity from 0.95 to 1.07.

Another embodiment comprises cyclosporin A, Dur-Em 207E®, and an oil having a specific gravity from 0.95 to 1.07.

Another embodiment comprises cyclosporin A, Emerest 2704®, and an oil having a specific gravity from 0.95 to 1.07.

Another embodiment comprises cyclosporin A, Emerest 2712®, and an oil having a specific gravity from 0.95 to 1.07.

Another embodiment comprises cyclosporin A, Emerest 2715®, and an oil having a specific gravity from 0.95 to 1.07.

Another embodiment comprises cyclosporin A, Ethal EH-2®, and an oil having a specific gravity from 0.95 to 1.07.

Another embodiment comprises cyclosporin A, Ethal EH-5®, and an oil having a specific gravity from 0.95 to 1.07.

Another embodiment comprises cyclosporin A, Ethal OA-23®, and an oil having a specific gravity from 0.95 to 1.07.

Another embodiment comprises cyclosporin A, Ethosperse CA-2O®, and an oil having a specific gravity from 0.95 to 1.07.

Another embodiment comprises cyclosporin A, Ethosperse LA-12®, and an oil having a specific gravity from 0.95 to 1.07.

Another embodiment comprises cyclosporin A, Ethosperse LA-23®, and an oil having a specific gravity from 0.95 to 1.07.

Another embodiment comprises cyclosporin A, Ethosperse LA-4®, and an oil having a specific gravity from 0.95 to 1.07.

Another embodiment comprises cyclosporin A, Ethox 3095®, and an oil having a specific gravity from 0.95 to 1.07.

Another embodiment comprises cyclosporin A, Ethox CO-16®, and an oil having a specific gravity from 0.95 to 1.07.

Another embodiment comprises cyclosporin A, Ethox CO-200®, and an oil having a specific gravity from 0.95 to 1.07.

Another embodiment comprises cyclosporin A, Ethox CO-25®, and an oil having a specific gravity from 0.95 to 1.07.

Another embodiment comprises cyclosporin A, Ethox CO-30®, and an oil having a specific gravity from 0.95 to 1.07.

Another embodiment comprises cyclosporin A, Ethox CO-36®, and an oil having a specific gravity from 0.95 to 1.07.

Another embodiment comprises cyclosporin A, Ethox CO-40®, and an oil having a specific gravity from 0.95 to 1.07.

Another embodiment comprises cyclosporin A, Ethox CO-5®, and an oil having a specific gravity from 0.95 to 1.07.

Another embodiment comprises cyclosporin A, Ethox DO-14®, and an oil having a specific gravity from 0.95 to 1.07.

Another embodiment comprises cyclosporin A, Ethox DO-9®, and an oil having a specific gravity from 0.95 to 1.07.

Another embodiment comprises cyclosporin A, Ethox MA-8®, and an oil having a specific gravity from 0.95 to 1.07.

Another embodiment comprises cyclosporin A, Ethox MI-14®, and an oil having a specific gravity from 0.95 to 1.07.

Another embodiment comprises cyclosporin A, Ethox ML-14®, and an oil having a specific gravity from 0.95 to 1.07.

Another embodiment comprises cyclosporin A, Ethox ML-5®, and an oil having a specific gravity from 0.95 to 1.07.

Another embodiment comprises cyclosporin A, Ethox MO-14®, and an oil having a specific gravity from 0.95 to 1.07.

Another embodiment comprises cyclosporin A, Ethox MO-9®, and an oil having a specific gravity from 0.95 to 1.07.

Another embodiment comprises cyclosporin A, Ethox MS-14®, and an oil having a specific gravity from 0.95 to 1.07.

Another embodiment comprises cyclosporin A, Ethox MS-23®, and an oil having a specific gravity from 0.95 to 1.07.

Another embodiment comprises cyclosporin A, Ethox MS-40®, and an oil having a specific gravity from 0.95 to 1.07.

Another embodiment comprises cyclosporin A, Ethox MS-8®, and an oil having a specific gravity from 0.95 to 1.07.

Another embodiment comprises cyclosporin A, Ethox TO-16®, and an oil having a specific gravity from 0.95 to 1.07.

Another embodiment comprises cyclosporin A, Ethox TO-8®, and an oil having a specific gravity from 0.95 to 1.07.

Another embodiment comprises cyclosporin A, Ethylan SN-120®, and an oil having a specific gravity from 0.95 to 1.07.

Another embodiment comprises cyclosporin A, Ethylan SN-70®, and an oil having a specific gravity from 0.95 to 1.07.

Another embodiment comprises cyclosporin A, Ethylan SN-90®, and an oil having a specific gravity from 0.95 to 1.07.

Another embodiment comprises cyclosporin A, Flexricin 13®, and an oil having a specific gravity from 0.95 to 1.07.

Another embodiment comprises cyclosporin A, Flexricin 15®, and an oil having a specific gravity from 0.95 to 1.07.

Another embodiment comprises cyclosporin A, Genapol 1261®, and an oil having a specific gravity from 0.95 to 1.07.

Another embodiment comprises cyclosporin A, Generol 122 E25®, and an oil having a specific gravity from 0.95 to 1.07.

Another embodiment comprises cyclosporin A, Glicepol 160®, and an oil having a specific gravity from 0.95 to 1.07.

Another embodiment comprises cyclosporin A, Glicepol 560®, and an oil having a specific gravity from 0.95 to 1.07.

Another embodiment comprises cyclosporin A, Glicepol GMS 20®, and an oil having a specific gravity from 0.95 to 1.07.

Another embodiment comprises cyclosporin A, Hetoxide BN-13®, and an oil having a specific gravity from 0.95 to 1.07.

Another embodiment comprises cyclosporin A, Hetoxide DNP-4®, and an oil having a specific gravity from 0.95 to 1.07.

Another embodiment comprises cyclosporin A, Hetoxide DNP-9.6®, and an oil having a specific gravity from 0.95 to 1.07.

Another embodiment comprises cyclosporin A, Hetoxide G-26®, and an oil having a specific gravity from 0.95 to 1.07.

Another embodiment comprises cyclosporin A, Hetoxide G-7®, and an oil having a specific gravity from 0.95 to 1.07.

Another embodiment comprises cyclosporin A, Hetoxide GT-20®, and an oil having a specific gravity from 0.95 to 1.07.

Another embodiment comprises cyclosporin A, Hetoxide HC-40®, and an oil having a specific gravity from 0.95 to 1.07.

Another embodiment comprises cyclosporin A, Hetoxol C-24®, and an oil having a specific gravity from 0.95 to 1.07.

Another embodiment comprises cyclosporin A, Hetoxol CA-10®, and an oil having a specific gravity from 0.95 to 1.07.

Another embodiment comprises cyclosporin A, Hetoxol CA-2®, and an oil having a specific gravity from 0.95 to 1.07.

Another embodiment comprises cyclosporin A, Hetoxol CA-20®, and an oil having a specific gravity from 0.95 to 1.07.

Another embodiment comprises cyclosporin A, Hetoxol CAWS®, and an oil having a specific gravity from 0.95 to 1.07.

Another embodiment comprises cyclosporin A, Hetoxol CD-3®, and an oil having a specific gravity from 0.95 to 1.07.

Another embodiment comprises cyclosporin A, Hetoxol CD-4®, and an oil having a specific gravity from 0.95 to 1.07.

Another embodiment comprises cyclosporin A, Hetoxol CS-15®, and an oil having a specific gravity from 0.95 to 1.07.

Another embodiment comprises cyclosporin A, Hetoxol CS-20®, and an oil having a specific gravity from 0.95 to 1.07.

Another embodiment comprises cyclosporin A, Hetoxol CS-20D®, and an oil having a specific gravity from 0.95 to 1.07.

Another embodiment comprises cyclosporin A, Hetoxol CS-25®, and an oil having a specific gravity from 0.95 to 1.07.

Another embodiment comprises cyclosporin A, Hetoxol CS-50®, and an oil having a specific gravity from 0.95 to 1.07.

Another embodiment comprises cyclosporin A, Hetoxol CS-9®, and an oil having a specific gravity from 0.95 to 1.07.

Another embodiment comprises cyclosporin A, Hetoxol CSA-15®, and an oil having a specific gravity from 0.95 to 1.07.

Another embodiment comprises cyclosporin A, Hetoxol D®, and an oil having a specific gravity from 0.95 to 1.07.

Another embodiment comprises cyclosporin A, Hetoxol G®, and an oil having a specific gravity from 0.95 to 1.07.

Another embodiment comprises cyclosporin A, Hetoxol J®, and an oil having a specific gravity from 0.95 to 1.07.

Another embodiment comprises cyclosporin A, Hetoxol L®, and an oil having a specific gravity from 0.95 to 1.07.

Another embodiment comprises cyclosporin A, Hetoxol L-23®, and an oil having a specific gravity from 0.95 to 1.07.

Another embodiment comprises cyclosporin A, Hetoxol L-4®, and an oil having a specific gravity from 0.95 to 1.07.

Another embodiment comprises cyclosporin A, Hetoxol L-9®, and an oil having a specific gravity from 0.95 to 1.07.

Another embodiment comprises cyclosporin A, Hetoxol LS-9®, and an oil having a specific gravity from 0.95 to 1.07.

Another embodiment comprises cyclosporin A, Hetoxol M-3®, and an oil having a specific gravity from 0.95 to 1.07.

Another embodiment comprises cyclosporin A, Hetoxol OA-10 Special®, and an oil having a specific gravity from 0.95 to 1.07.

Another embodiment comprises cyclosporin A, Hetoxol OA-20 Special®, and an oil having a specific gravity from 0.95 to 1.07.

Another embodiment comprises cyclosporin A, Hetoxol OA-3 Special®, and an oil having a specific gravity from 0.95 to 1.07.

Another embodiment comprises cyclosporin A, Hetoxol OA-5 Special®, and an oil having a specific gravity from 0.95 to 1.07.

Another embodiment comprises cyclosporin A, Hetoxol OL-10®, and an oil having a specific gravity from 0.95 to 1.07.

Another embodiment comprises cyclosporin A, Hetoxol OL-2®, and an oil having a specific gravity from 0.95 to 1.07.

Another embodiment comprises cyclosporin A, Hetoxol OL-23®, and an oil having a specific gravity from 0.95 to 1.07.

Another embodiment comprises cyclosporin A, Hetoxol OL-4®, and an oil having a specific gravity from 0.95 to 1.07.

Another embodiment comprises cyclosporin A, Hetoxol OL-40®, and an oil having a specific gravity from 0.95 to 1.07.

Another embodiment comprises cyclosporin A, Hetoxol OL-5®, and an oil having a specific gravity from 0.95 to 1.07.

Another embodiment comprises cyclosporin A, Hetoxol PLA®, and an oil having a specific gravity from 0.95 to 1.07.

Another embodiment comprises cyclosporin A, Hetoxol SP-15®, and an oil having a specific gravity from 0.95 to 1.07.

Another embodiment comprises cyclosporin A, Hetoxol STA-10®, and an oil having a specific gravity from 0.95 to 1.07.

Another embodiment comprises cyclosporin A, Hetoxol STA-2®, and an oil having a specific gravity from 0.95 to 1.07.

Another embodiment comprises cyclosporin A, Hetoxol STA-30®, and an oil having a specific gravity from 0.95 to 1.07.

Another embodiment comprises cyclosporin A, Hetoxol TD-12®, and an oil having a specific gravity from 0.95 to 1.07.

Another embodiment comprises cyclosporin A, Hetoxol TD-18®, and an oil having a specific gravity from 0.95 to 1.07.

Another embodiment comprises cyclosporin A, Hetoxol TD-3®, and an oil having a specific gravity from 0.95 to 1.07.

Another embodiment comprises cyclosporin A, Hetoxol TD-6®, and an oil having a specific gravity from 0.95 to 1.07.

Another embodiment comprises cyclosporin A, Hetoxol TD-9®, and an oil having a specific gravity from 0.95 to 1.07.

Another embodiment comprises cyclosporin A, Hetoxol TDEP-15®, and an oil having a specific gravity from 0.95 to 1.07.

Another embodiment comprises cyclosporin A, Hetoxol TDEP-63®, and an oil having a specific gravity from 0.95 to 1.07.

Another embodiment comprises cyclosporin A, Iconol 24-12®, and an oil having a specific gravity from 0.95 to 1.07.

Another embodiment comprises cyclosporin A, Iconol 24-23®, and an oil having a specific gravity from 0.95 to 1.07.

Another embodiment comprises cyclosporin A, Iconol 24-4®, and an oil having a specific gravity from 0.95 to 1.07.

Another embodiment comprises cyclosporin A, Iconol DA-4®, and an oil having a specific gravity from 0.95 to 1.07.

Another embodiment comprises cyclosporin A, Iconol DA-6®, and an oil having a specific gravity from 0.95 to 1.07.

Another embodiment comprises cyclosporin A, Iconol TDA-10®, and an oil having a specific gravity from 0.95 to 1.07.

Another embodiment comprises cyclosporin A, Iconol TDA-3®, and an oil having a specific gravity from 0.95 to 1.07.

Another embodiment comprises cyclosporin A, Iconol TDA-6®, and an oil having a specific gravity from 0.95 to 1.07.

Another embodiment comprises cyclosporin A, Iconol TDA-8-90%®, and an oil having a specific gravity from 0.95 to 1.07.

Another embodiment comprises cyclosporin A, Iconol TDA-9®, and an oil having a specific gravity from 0.95 to 1.07.

Another embodiment comprises cyclosporin A, Igepal CA-210®, and an oil having a specific gravity from 0.95 to 1.07.

Another embodiment comprises cyclosporin A, Igepal CA-407®, and an oil having a specific gravity from 0.95 to 1.07.

Another embodiment comprises cyclosporin A, Igepal CA-420®, and an oil having a specific gravity from 0.95 to 1.07.

Another embodiment comprises cyclosporin A, Igepal CA-520®, and an oil having a specific gravity from 0.95 to 1.07.

Another embodiment comprises cyclosporin A, Igepal CA-620®, and an oil having a specific gravity from 0.95 to 1.07.

Another embodiment comprises cyclosporin A, Igepal CA-630®, and an oil having a specific gravity from 0.95 to 1.07.

Another embodiment comprises cyclosporin A, Igepal CA-720®, and an oil having a specific gravity from 0.95 to 1.07.

Another embodiment comprises cyclosporin A, Igepal CA-877®, and an oil having a specific gravity from 0.95 to 1.07.

Another embodiment comprises cyclosporin A, Igepal CA-887®, and an oil having a specific gravity from 0.95 to 1.07.

Another embodiment comprises cyclosporin A, Igepal CA-897®, and an oil having a specific gravity from 0.95 to 1.07.

Another embodiment comprises cyclosporin A, Igepal CO-210®, and an oil having a specific gravity from 0.95 to 1.07.

Another embodiment comprises cyclosporin A, Igepal CO-430®, and an oil having a specific gravity from 0.95 to 1.07.

Another embodiment comprises cyclosporin A, Igepal CO-520®, and an oil having a specific gravity from 0.95 to 1.07.

Another embodiment comprises cyclosporin A, Igepal CO-530®, and an oil having a specific gravity from 0.95 to 1.07.

Another embodiment comprises cyclosporin A, Igepal CO-610®, and an oil having a specific gravity from 0.95 to 1.07.

Another embodiment comprises cyclosporin A, Igepal CO-630®, and an oil having a specific gravity from 0.95 to 1.07.

Another embodiment comprises cyclosporin A, Igepal CO-630 Special®, and an oil having a specific gravity from 0.95 to 1.07.

Another embodiment comprises cyclosporin A, Igepal CO-660®, and an oil having a specific gravity from 0.95 to 1.07.

Another embodiment comprises cyclosporin A, Igepal CO-710®, and an oil having a specific gravity from 0.95 to 1.07.

Another embodiment comprises cyclosporin A, Igepal CO-720®, and an oil having a specific gravity from 0.95 to 1.07.

Another embodiment comprises cyclosporin A, Igepal CO-730®, and an oil having a specific gravity from 0.95 to 1.07.

Another embodiment comprises cyclosporin A, Igepal CO-738®, and an oil having a specific gravity from 0.95 to 1.07.

Another embodiment comprises cyclosporin A, Igepal CO-850®, and an oil having a specific gravity from 0.95 to 1.07.

Another embodiment comprises cyclosporin A, Igepal CO-880®, and an oil having a specific gravity from 0.95 to 1.07.

Another embodiment comprises cyclosporin A, Igepal CO-887®, and an oil having a specific gravity from 0.95 to 1.07.

Another embodiment comprises cyclosporin A, Igepal CO-890®, and an oil having a specific gravity from 0.95 to 1.07.

Another embodiment comprises cyclosporin A, Igepal CO-897®, and an oil having a specific gravity from 0.95 to 1.07.

Another embodiment comprises cyclosporin A, Igepal CO-970®, and an oil having a specific gravity from 0.95 to 1.07.

Another embodiment comprises cyclosporin A, Igepal CO-977®, and an oil having a specific gravity from 0.95 to 1.07.

Another embodiment comprises cyclosporin A, Igepal CO-987®, and an oil having a specific gravity from 0.95 to 1.07.

Another embodiment comprises cyclosporin A, Igepal CO-990 FLK®, and an oil having a specific gravity from 0.95 to 1.07.

Another embodiment comprises cyclosporin A, Igepal CO-997®, and an oil having a specific gravity from 0.95 to 1.07.

Another embodiment comprises cyclosporin A, Igepal DM-430®, and an oil having a specific gravity from 0.95 to 1.07.

Another embodiment comprises cyclosporin A, Igepal DM-530®, and an oil having a specific gravity from 0.95 to 1.07.

Another embodiment comprises cyclosporin A, Igepal DM-710®, and an oil having a specific gravity from 0.95 to 1.07.

Another embodiment comprises cyclosporin A, Igepal DM-730®, and an oil having a specific gravity from 0.95 to 1.07.

Another embodiment comprises cyclosporin A, Igepal DM-880®, and an oil having a specific gravity from 0.95 to 1.07.

Another embodiment comprises cyclosporin A, Igepal DM-970 FLK®, and an oil having a specific gravity from 0.95 to 1.07.

Another embodiment comprises cyclosporin A, Igepal OD-410®, and an oil having a specific gravity from 0.95 to 1.07.

Another embodiment comprises cyclosporin A, Igepal RC-520®, and an oil having a specific gravity from 0.95 to 1.07.

Another embodiment comprises cyclosporin A, Igepal RC-620®, and an oil having a specific gravity from 0.95 to 1.07.

Another embodiment comprises cyclosporin A, Igepal RC-630®, and an oil having a specific gravity from 0.95 to 1.07.

Another embodiment comprises cyclosporin A, Lexemul CS-20®, and an oil having a specific gravity from 0.95 to 1.07.

Another embodiment comprises cyclosporin A, Lipo GMS 450®, and an oil having a specific gravity from 0.95 to 1.07.

Another embodiment comprises cyclosporin A, Lipo Polyglycol 1000®, and an oil having a specific gravity from 0.95 to 1.07.

Another embodiment comprises cyclosporin A, Lipo Polyglycol 200®, and an oil having a specific gravity from 0.95 to 1.07.

Another embodiment comprises cyclosporin A, Lipo Polyglycol 300®, and an oil having a specific gravity from 0.95 to 1.07.

Another embodiment comprises cyclosporin A, Lipo Polyglycol 3350®, and an oil having a specific gravity from 0.95 to 1.07.

Another embodiment comprises cyclosporin A, Lipo Polyglycol 400®, and an oil having a specific gravity from 0.95 to 1.07.

Another embodiment comprises cyclosporin A, Lipo Polyglycol 600®, and an oil having a specific gravity from 0.95 to 1.07.

Another embodiment comprises cyclosporin A, Lipocol C-10®, and an oil having a specific gravity from 0.95 to 1.07.

Another embodiment comprises cyclosporin A, Lipocol C-2®, and an oil having a specific gravity from 0.95 to 1.07.

Another embodiment comprises cyclosporin A, Lipocol C-20®, and an oil having a specific gravity from 0.95 to 1.07.

Another embodiment comprises cyclosporin A, Lipocol L-12®, and an oil having a specific gravity from 0.95 to 1.07.

Another embodiment comprises cyclosporin A, Lipocol L-23®, and an oil having a specific gravity from 0.95 to 1.07.

Another embodiment comprises cyclosporin A, Lipocol L-4®, and an oil having a specific gravity from 0.95 to 1.07.

Another embodiment comprises cyclosporin A, Lipocol O-10®, and an oil having a specific gravity from 0.95 to 1.07.

Another embodiment comprises cyclosporin A, Lipocol O-2®, and an oil having a specific gravity from 0.95 to 1.07.

Another embodiment comprises cyclosporin A, Lipocol O-20®, and an oil having a specific gravity from 0.95 to 1.07.

Another embodiment comprises cyclosporin A, Lipocol O-5®, and an oil having a specific gravity from 0.95 to 1.07.

Another embodiment comprises cyclosporin A, Lipocol S-10®, and an oil having a specific gravity from 0.95 to 1.07.

Another embodiment comprises cyclosporin A, Lipocol S-2®, and an oil having a specific gravity from 0.95 to 1.07.

Another embodiment comprises cyclosporin A, Lipocol S-20®, and an oil having a specific gravity from 0.95 to 1.07.

Another embodiment comprises cyclosporin A, Lipocol SC-15®, and an oil having a specific gravity from 0.95 to 1.07.

Another embodiment comprises cyclosporin A, Lipocol SC-20®, and an oil having a specific gravity from 0.95 to 1.07.

Another embodiment comprises cyclosporin A, Lipocol SC-4®, and an oil having a specific gravity from 0.95 to 1.07.

Another embodiment comprises cyclosporin A, Lipocol TD-12®, and an oil having a specific gravity from 0.95 to 1.07.

Another embodiment comprises cyclosporin A, Lumulse 40-O K®, and an oil having a specific gravity from 0.95 to 1.07.

Another embodiment comprises cyclosporin A, Lumulse 42-O K®, and an oil having a specific gravity from 0.95 to 1.07.

Another embodiment comprises cyclosporin A, Lumulse GMT-40®, and an oil having a specific gravity from 0.95 to 1.07.

Another embodiment comprises cyclosporin A, Lumulse PEG 1450®, and an oil having a specific gravity from 0.95 to 1.07.

Another embodiment comprises cyclosporin A, Lumulse PEG 1450 NF®, and an oil having a specific gravity from 0.95 to 1.07.

Another embodiment comprises cyclosporin A, Lumulse PEG 8000®, and an oil having a specific gravity from 0.95 to 1.07.

Another embodiment comprises cyclosporin A, Lumulse PGO®, and an oil having a specific gravity from 0.95 to 1.07.

Another embodiment comprises cyclosporin A, Lumulse POE (2) Oleyl Amine®, and an oil having a specific gravity from 0.95 to 1.07.

Another embodiment comprises cyclosporin A, Magrabar PGO-104®, and an oil having a specific gravity from 0.95 to 1.07.

Another embodiment comprises cyclosporin A, Mapeg 200 ML®, and an oil having a specific gravity from 0.95 to 1.07.

Another embodiment comprises cyclosporin A, Mapeg 400 MO®, and an oil having a specific gravity from 0.95 to 1.07.

Another embodiment comprises cyclosporin A, Mapeg 400 MOT®, and an oil having a specific gravity from 0.95 to 1.07.

Another embodiment comprises cyclosporin A, Mapeg 400DO®, and an oil having a specific gravity from 0.95 to 1.07.

Another embodiment comprises cyclosporin A, Mapeg 400DOT®, and an oil having a specific gravity from 0.95 to 1.07.

Another embodiment comprises cyclosporin A, Mapeg 600DOT®, and an oil having a specific gravity from 0.95 to 1.07.

Another embodiment comprises cyclosporin A, Marlox B 24/80®, and an oil having a specific gravity from 0.95 to 1.07.

Another embodiment comprises cyclosporin A, Marlox FK 86®, and an oil having a specific gravity from 0.95 to 1.07.

Another embodiment comprises cyclosporin A, Marlox MO 154®, and an oil having a specific gravity from 0.95 to 1.07.

Another embodiment comprises cyclosporin A, Mazox CDA CG®, and an oil having a specific gravity from 0.95 to 1.07.

Another embodiment comprises cyclosporin A, Monoglycerides Glyceryl Monestearate®, and an oil having a specific gravity from 0.95 to 1.07.

Another embodiment comprises cyclosporin A, Myrj 45®, and an oil having a specific gravity from 0.95 to 1.07.

Another embodiment comprises cyclosporin A, Myrj 52®, and an oil having a specific gravity from 0.95 to 1.07.

Another embodiment comprises cyclosporin A, Myrj 52S®, and an oil having a specific gravity from 0.95 to 1.07.

Another embodiment comprises cyclosporin A, Myrj 53®, and an oil having a specific gravity from 0.95 to 1.07.

Another embodiment comprises cyclosporin A, Myrj 59®, and an oil having a specific gravity from 0.95 to 1.07.

Another embodiment comprises cyclosporin A, Norfox NP-12®, and an oil having a specific gravity from 0.95 to 1.07.

Another embodiment comprises cyclosporin A, Norfox NP-4®, and an oil having a specific gravity from 0.95 to 1.07.

Another embodiment comprises cyclosporin A, Norfox NP-6®, and an oil having a specific gravity from 0.95 to 1.07.

Another embodiment comprises cyclosporin A, Norfox NP-887®, and an oil having a specific gravity from 0.95 to 1.07.

Another embodiment comprises cyclosporin A, Norfox NP-9®, and an oil having a specific gravity from 0.95 to 1.07.

Another embodiment comprises cyclosporin A, Norfox NP-977®, and an oil having a specific gravity from 0.95 to 1.07.

Another embodiment comprises cyclosporin A, Norfox OP-100®, and an oil having a specific gravity from 0.95 to 1.07.

Another embodiment comprises cyclosporin A, Norfox OP-102®, and an oil having a specific gravity from 0.95 to 1.07.

Another embodiment comprises cyclosporin A, Norfox OP-114®, and an oil having a specific gravity from 0.95 to 1.07.

Another embodiment comprises cyclosporin A, Norfox OP-45®, and an oil having a specific gravity from 0.95 to 1.07.

Another embodiment comprises cyclosporin A, Pegosperse 100-L®, and an oil having a specific gravity from 0.95 to 1.07.

Another embodiment comprises cyclosporin A, Pegosperse 100-O®, and an oil having a specific gravity from 0.95 to 1.07.

Another embodiment comprises cyclosporin A, Pegosperse 100-S®, and an oil having a specific gravity from 0.95 to 1.07.

Another embodiment comprises cyclosporin A, Pegosperse 1500-MS®, and an oil having a specific gravity from 0.95 to 1.07.

Another embodiment comprises cyclosporin A, Pegosperse 1750-MS®, and an oil having a specific gravity from 0.95 to 1.07.

Another embodiment comprises cyclosporin A, Pegosperse 200 DL®, and an oil having a specific gravity from 0.95 to 1.07.

Another embodiment comprises cyclosporin A, Pegosperse 200-ML®, and an oil having a specific gravity from 0.95 to 1.07.

Another embodiment comprises cyclosporin A, Pegosperse 400-DL®, and an oil having a specific gravity from 0.95 to 1.07.

Another embodiment comprises cyclosporin A, Pegosperse 400-DO®, and an oil having a specific gravity from 0.95 to 1.07.

Another embodiment comprises cyclosporin A, Pegosperse 400-DS®, and an oil having a specific gravity from 0.95 to 1.07.

Another embodiment comprises cyclosporin A, Pegosperse 400-ML®, and an oil having a specific gravity from 0.95 to 1.07.

Another embodiment comprises cyclosporin A, Pegosperse 400-MO®, and an oil having a specific gravity from 0.95 to 1.07.

Another embodiment comprises cyclosporin A, Pegosperse 400-MOT®, and an oil having a specific gravity from 0.95 to 1.07.

Another embodiment comprises cyclosporin A, Pegosperse 400-MS®, and an oil having a specific gravity from 0.95 to 1.07.

Another embodiment comprises cyclosporin A, Pegosperse 50-DS®, and an oil having a specific gravity from 0.95 to 1.07.

Another embodiment comprises cyclosporin A, Pegosperse 50-MS®, and an oil having a specific gravity from 0.95 to 1.07.

Another embodiment comprises cyclosporin A, Pegosperse 600-ML®, and an oil having a specific gravity from 0.95 to 1.07.

Another embodiment comprises cyclosporin A, Pegosperse 600-MS®, and an oil having a specific gravity from 0.95 to 1.07.

Another embodiment comprises cyclosporin A, Plurafac A-38®, and an oil having a specific gravity from 0.95 to 1.07.

Another embodiment comprises cyclosporin A, Plurafac B-25-5®, and an oil having a specific gravity from 0.95 to 1.07.

Another embodiment comprises cyclosporin A, Plurafac B-26®, and an oil having a specific gravity from 0.95 to 1.07.

Another embodiment comprises cyclosporin A, Plurafac D-25®, and an oil having a specific gravity from 0.95 to 1.07.

Another embodiment comprises cyclosporin A, Plurafac LF 131®, and an oil having a specific gravity from 0.95 to 1.07.

Another embodiment comprises cyclosporin A, Plurafac LF 4030®, and an oil having a specific gravity from 0.95 to 1.07.

Another embodiment comprises cyclosporin A, Plurafac LF 7000®, and an oil having a specific gravity from 0.95 to 1.07.

Another embodiment comprises cyclosporin A, Plurafac RA-20®, and an oil having a specific gravity from 0.95 to 1.07.

Another embodiment comprises cyclosporin A, Plurafac RA-30®, and an oil having a specific gravity from 0.95 to 1.07.

Another embodiment comprises cyclosporin A, Plurafac RA-40®, and an oil having a specific gravity from 0.95 to 1.07.

Another embodiment comprises cyclosporin A, Pluronic 10R5®, and an oil having a specific gravity from 0.95 to 1.07.

Another embodiment comprises cyclosporin A, Pluronic 17R2®, and an oil having a specific gravity from 0.95 to 1.07.

Another embodiment comprises cyclosporin A, Pluronic 17R4®, and an oil having a specific gravity from 0.95 to 1.07.

Another embodiment comprises cyclosporin A, Pluronic 25R2®, and an oil having a specific gravity from 0.95 to 1.07.

Another embodiment comprises cyclosporin A, Pluronic 25R4®, and an oil having a specific gravity from 0.95 to 1.07.

Another embodiment comprises cyclosporin A, Pluronic 31R1®, and an oil having a specific gravity from 0.95 to 1.07.

Another embodiment comprises cyclosporin A, Pluronic F108®, and an oil having a specific gravity from 0.95 to 1.07.

Another embodiment comprises cyclosporin A, Pluronic F127®, and an oil having a specific gravity from 0.95 to 1.07.

Another embodiment comprises cyclosporin A, Pluronic F38®, and an oil having a specific gravity from 0.95 to 1.07.

Another embodiment comprises cyclosporin A, Pluronic F68®, and an oil having a specific gravity from 0.95 to 1.07.

Another embodiment comprises cyclosporin A, Pluronic F68LF®, and an oil having a specific gravity from 0.95 to 1.07.

Another embodiment comprises cyclosporin A, Pluronic F77®, and an oil having a specific gravity from 0.95 to 1.07.

Another embodiment comprises cyclosporin A, Pluronic F87®, and an oil having a specific gravity from 0.95 to 1.07.

Another embodiment comprises cyclosporin A, Pluronic F88®, and an oil having a specific gravity from 0.95 to 1.07.

Another embodiment comprises cyclosporin A, Pluronic F98®, and an oil having a specific gravity from 0.95 to 1.07.

Another embodiment comprises cyclosporin A, Pluronic L10®, and an oil having a specific gravity from 0.95 to 1.07.

Another embodiment comprises cyclosporin A, Pluronic L101®, and an oil having a specific gravity from 0.95 to 1.07.

Another embodiment comprises cyclosporin A, Pluronic L121®, and an oil having a specific gravity from 0.95 to 1.07.

Another embodiment comprises cyclosporin A, Pluronic L31®, and an oil having a specific gravity from 0.95 to 1.07.

Another embodiment comprises cyclosporin A, Pluronic L35®, and an oil having a specific gravity from 0.95 to 1.07.

Another embodiment comprises cyclosporin A, Pluronic L43®, and an oil having a specific gravity from 0.95 to 1.07.

Another embodiment comprises cyclosporin A, Pluronic L44®, and an oil having a specific gravity from 0.95 to 1.07.

Another embodiment comprises cyclosporin A, Pluronic L61®, and an oil having a specific gravity from 0.95 to 1.07.

Another embodiment comprises cyclosporin A, Pluronic L62®, and an oil having a specific gravity from 0.95 to 1.07.

Another embodiment comprises cyclosporin A, Pluronic L62D®, and an oil having a specific gravity from 0.95 to 1.07.

Another embodiment comprises cyclosporin A, Pluronic L62LF®, and an oil having a specific gravity from 0.95 to 1.07.

Another embodiment comprises cyclosporin A, Pluronic L64®, and an oil having a specific gravity from 0.95 to 1.07.

Another embodiment comprises cyclosporin A, Pluronic L81®, and an oil having a specific gravity from 0.95 to 1.07.

Another embodiment comprises cyclosporin A, Pluronic L92®, and an oil having a specific gravity from 0.95 to 1.07.

Another embodiment comprises cyclosporin A, Pluronic P103®, and an oil having a specific gravity from 0.95 to 1.07.

Another embodiment comprises cyclosporin A, Pluronic P104®, and an oil having a specific gravity from 0.95 to 1.07.

Another embodiment comprises cyclosporin A, Pluronic P105®, and an oil having a specific gravity from 0.95 to 1.07.

Another embodiment comprises cyclosporin A, Pluronic P123®, and an oil having a specific gravity from 0.95 to 1.07.

Another embodiment comprises cyclosporin A, Pluronic P65®, and an oil having a specific gravity from 0.95 to 1.07.

Another embodiment comprises cyclosporin A, Pluronic P84®, and an oil having a specific gravity from 0.95 to 1.07.

Another embodiment comprises cyclosporin A, Pluronic P85®, and an oil having a specific gravity from 0.95 to 1.07.

Another embodiment comprises cyclosporin A, Polyaldo 10-2-P®, and an oil having a specific gravity from 0.95 to 1.

Another embodiment comprises cyclosporin A, Polyaldo DGDO®, and an oil having a specific gravity from 0.95 to 1.07.

Another embodiment comprises cyclosporin A, Polyaldo HGDS®, and an oil having a specific gravity from 0.95 to 1.07.

Another embodiment comprises cyclosporin A, Polyaldo TGMS®, and an oil having a specific gravity from 0.95 to 1.07.

Another embodiment comprises cyclosporin A, Protegin V®, and an oil having a specific gravity from 0.95 to 1.07.

Another embodiment comprises cyclosporin A, Protegin W®, and an oil having a specific gravity from 0.95 to 1.07.

Another embodiment comprises cyclosporin A, Protegin WX®, and an oil having a specific gravity from 0.95 to 1.07.

Another embodiment comprises cyclosporin A, Protegin XV®, and an oil having a specific gravity from 0.95 to 1.07.

Another embodiment comprises cyclosporin A, Rhodasurf BC-420®, and an oil having a specific gravity from 0.95 to 1.07.

Another embodiment comprises cyclosporin A, Rhodasurf BC-610®, and an oil having a specific gravity from 0.95 to 1.07.

Another embodiment comprises cyclosporin A, Rhodasurf BC-630®, and an oil having a specific gravity from 0.95 to 1.07.

Another embodiment comprises cyclosporin A, Rhodasurf BC-720®, and an oil having a specific gravity from 0.95 to 1.07.

Another embodiment comprises cyclosporin A, Rhodasurf BC-840®, and an oil having a specific gravity from 0.95 to 1.07.

Another embodiment comprises cyclosporin A, Rhodasurf L 1®, and an oil having a specific gravity from 0.95 to 1.07.

Another embodiment comprises cyclosporin A, Rhodasurf L 12®, and an oil having a specific gravity from 0.95 to 1.07.

Another embodiment comprises cyclosporin A, Rhodasurf L 2®, and an oil having a specific gravity from 0.95 to 1.07.

Another embodiment comprises cyclosporin A, Rhodasurf L 20®, and an oil having a specific gravity from 0.95 to 1.07.

Another embodiment comprises cyclosporin A, Rhodasurf L 3®, and an oil having a specific gravity from 0.95 to 1.07.

Another embodiment comprises cyclosporin A, Rhodasurf L 30®, and an oil having a specific gravity from 0.95 to 1.07.

Another embodiment comprises cyclosporin A, Rhodasurf L 4®, and an oil having a specific gravity from 0.95 to 1.07.

Another embodiment comprises cyclosporin A, Rhodasurf L 50®, and an oil having a specific gravity from 0.95 to 1.07.

Another embodiment comprises cyclosporin A, Rhodasurf L 7/90®, and an oil having a specific gravity from 0.95 to 1.07.

Another embodiment comprises cyclosporin A, Rhodasurf LA-3®, and an oil having a specific gravity from 0.95 to 1.07.

Another embodiment comprises cyclosporin A, Rhodasurf LA-7®, and an oil having a specific gravity from 0.95 to 1.07.

Another embodiment comprises cyclosporin A, Rhodasurf LAN-23®, and an oil having a specific gravity from 0.95 to 1.07.

Another embodiment comprises cyclosporin A, Rhodasurf ON-870®, and an oil having a specific gravity from 0.95 to 1.07.

Another embodiment comprises cyclosporin A, Rhodasurf ON-877®, and an oil having a specific gravity from 0.95 to 1.07.

Another embodiment comprises cyclosporin A, Rhodasurf TDA-8/5®, and an oil having a specific gravity from 0.95 to 1.07.

Another embodiment comprises cyclosporin A, Ritapro 100®, and an oil having a specific gravity from 0.95 to 1.07.

Another embodiment comprises cyclosporin A, Ritapro 165®, and an oil having a specific gravity from 0.95 to 1.07.

Another embodiment comprises cyclosporin A, Ritapro 200®, and an oil having a specific gravity from 0.95 to 1.07.

Another embodiment comprises cyclosporin A, Ritapro 300®, and an oil having a specific gravity from 0.95 to 1.07.

Another embodiment comprises cyclosporin A, Ritoleth 10®, and an oil having a specific gravity from 0.95 to 1.07.

Another embodiment comprises cyclosporin A, Ritoleth 2®, and an oil having a specific gravity from 0.95 to 1.07.

Another embodiment comprises cyclosporin A, Ritoleth 20®, and an oil having a specific gravity from 0.95 to 1.07.

Another embodiment comprises cyclosporin A, Ritoleth 5®, and an oil having a specific gravity from 0.95 to 1.07.

Another embodiment comprises cyclosporin A, Santone 10-10-0®, and an oil having a specific gravity from 0.95 to 1.07.

Another embodiment comprises cyclosporin A, Santone 3-1-S XTR®, and an oil having a specific gravity from 0.95 to 1.07.

Another embodiment comprises cyclosporin A, Santone 3-1-SH®, and an oil having a specific gravity from 0.95 to 1.07.

Another embodiment comprises cyclosporin A, Santone 8-1-O®, and an oil having a specific gravity from 0.95 to 1.07.

Another embodiment comprises cyclosporin A, Solulan 16®, and an oil having a specific gravity from 0.95 to 1.07.

Another embodiment comprises cyclosporin A, Solulan 75®, and an oil having a specific gravity from 0.95 to 1.07.

Another embodiment comprises cyclosporin A, Solulan C-24®, and an oil having a specific gravity from 0.95 to 1.07.

Another embodiment comprises cyclosporin A, Solulan L-575®, and an oil having a specific gravity from 0.95 to 1.07.

Another embodiment comprises cyclosporin A, Standamox CAW®, and an oil having a specific gravity from 0.95 to 1.07.

Another embodiment comprises cyclosporin A, Surfine AZI-A®, and an oil having a specific gravity from 0.95 to 1.07.

Another embodiment comprises cyclosporin A, Surfonic CO-15®, and an oil having a specific gravity from 0.95 to 1.07.

Another embodiment comprises cyclosporin A, Surfonic CO-25®, and an oil having a specific gravity from 0.95 to 1.07.

Another embodiment comprises cyclosporin A, Surfonic CO-30®, and an oil having a specific gravity from 0.95 to 1.07.

Another embodiment comprises cyclospprin A, Surfonic CO-36®, and an oil having a specific gravity from 0.95 to 1.07.

Another embodiment comprises cyclosporin A, Surfonic CO-42®, and an oil having a specific gravity from 0.95 to 1.07.

Another embodiment comprises cyclosporin A, Surfonic E400-MO®, and an oil having a specific gravity from 0.95 to 1.07.

Another embodiment comprises cyclosporin A, Surfonic L12-3®, and an oil having a specific gravity from 0.95 to 1.07.

Another embodiment comprises cyclosporin A, Surfonic L12-6®, and an oil having a specific gravity from 0.95 to 1.07.

Another embodiment comprises cyclosporin A, Surfonic L24-8®, and an oil having a specific gravity from 0.95 to 1.07.

Another embodiment comprises cyclosporin A, Surfonic L24-12®, and an oil having a specific gravity from 0.95 to 1.07.

Another embodiment comprises cyclosporin A, Surfonic L24-2®, and an oil having a specific gravity from 0.95 to 1.07.

Another embodiment comprises cyclosporin A, Surfonic L24-22®, and an oil having a specific gravity from 0.95 to 1.07.

Another embodiment comprises cyclosporin A, Surfonic L24-3®, and an oil having a specific gravity from 0.95 to 1.07.

Another embodiment comprises cyclosporin A, Surfonic L24-4®, and an oil having a specific gravity from 0.95 to 1.07.

Another embodiment comprises cyclosporin A, Surfonic L24-5®, and an oil having a specific gravity from 0.95 to 1.07.

Another embodiment comprises cyclosporin A, Surfonic L24-7®, and an oil having a specific gravity from 0.95 to 1.07.

Another embodiment comprises cyclosporin A, Surfonic L24-7.1®, and an oil having a specific gravity from 0.95 to 1.07.

Another embodiment comprises cyclosporin A, Surfonic L24-9®, and an oil having a specific gravity from 0.95 to 1.07.

Another embodiment comprises cyclosporin A, Surfonic LF-17®, and an oil having a specific gravity from 0.95 to 1.07.

Another embodiment comprises cyclosporin A, Surfonic LF-18®, and an oil having a specific gravity from 0.95 to 1.07.

Another embodiment comprises cyclosporin A, Surfonic LF-37®, and an oil having a specific gravity from 0.95 to 1.07.

Another embodiment comprises cyclosporin A, Surfonic LF-41®, and an oil having a specific gravity from 0.95 to 1.07.

Another embodiment comprises cyclosporin A, Surfonic LF-42®, and an oil having a specific gravity from 0.95 to 1.07.

Another embodiment comprises cyclosporin A, Surfonic LF-47®, and an oil having a specific gravity from 0.95 to 1.07.

Another embodiment comprises cyclosporin A, Surfonic LF-68®, and an oil having a specific gravity from 0.95 to 1.07.

Another embodiment comprises cyclosporin A, Surfonic N-10®, and an oil having a specific gravity from 0.95 to 1.07.

Another embodiment comprises cyclosporin A, Surfonic N-100®, and an oil having a specific gravity from 0.95 to 1.07.

Another embodiment comprises cyclosporin A, Surfonic N-102®, and an oil having a specific gravity from 0.95 to 1.07.

Another embodiment comprises cyclosporin A, Surfonic N-120®, and an oil having a specific gravity from 0.95 to 1.07.

Another embodiment comprises cyclosporin A, Surfonic N-150®, and an oil having a specific gravity from 0.95 to 1.07.

Another embodiment comprises cyclosporin A, Surfonic N-200®, and an oil having a specific gravity from 0.95 to 1.07.

Another embodiment comprises cyclosporin A, Surfonic N-300®, and an oil having a specific gravity from 0.95 to 1.07.

Another embodiment comprises cyclosporin A, Surfonic N-31.5®, and an oil having a specific gravity from 0.95 to 1.07.

Another embodiment comprises cyclosporin A, Surfonic N-40®, and an oil having a specific gravity from 0.95 to 1.07.

Another embodiment comprises cyclosporin A, Surfonic N-400®, and an oil having a specific gravity from 0.95 to 1.07.

Another embodiment comprises cyclosporin A, Surfonic N-60®, and an oil having a specific gravity from 0.95 to 1.07.

Another embodiment comprises cyclosporin A, Surfonic N-85®, and an oil having a specific gravity from 0.95 to 1.07.

Another embodiment comprises cyclosporin A, Surfonic N-95®, and an oil having a specific gravity from 0.95 to 1.07.

Another embodiment comprises cyclosporin A, Surfonic NB-18®, and an oil having a specific gravity from 0.95 to 1.07.

Another embodiment comprises cyclosporin A, Surfonic NB-23®, and an oil having a specific gravity from 0.95 to 1.07.

Another embodiment comprises cyclosporin A, Surfonic NB-25®, and an oil having a specific gravity from 0.95 to 1.07.

Another embodiment comprises cyclosporin A, Surfonic NB-307®, and an oil having a specific gravity from 0.95 to 1.07.

Another embodiment comprises cyclosporin A, Surfonic NB-31®, and an oil having a specific gravity from 0.95 to 1.07.

Another embodiment comprises cyclosporin A, Surfonic NB-41®, and an oil having a specific gravity from 0.95 to 1.07.

Another embodiment comprises cyclosporin A, Surfonic NB-43®, and an oil having a specific gravity from 0.95 to 1.07.

Another embodiment comprises cyclosporin A, Surfonic OP-100®, and an oil having a specific gravity from 0.95 to 1.07.

Another embodiment comprises cyclosporin A, Surfonic OP-120®, and an oil having a specific gravity from 0.95 to 1.07.

Another embodiment comprises cyclosporin A, Surfonic OP-15®, and an oil having a specific gravity from 0.95 to 1.07.

Another embodiment comprises cyclosporin A, Surfonic OP-35®, and an oil having a specific gravity from 0.95 to 1.07.

Another embodiment comprises cyclosporin A, Surfonic OP-70®, and an oil having a specific gravity from 0.95 to 1.07.

Another embodiment comprises cyclosporin A, Surfonic P1®, and an oil having a specific gravity from 0.95 to 1.07.

Another embodiment comprises cyclosporin A, Surfonic P3®, and an oil having a specific gravity from 0.95 to 1.07.

Another embodiment comprises cyclosporin A, Surfonic P5®, and an oil having a specific gravity from 0.95 to 1.07.

Another embodiment comprises cyclosporin A, Surfonic P6®, and an oil having a specific gravity from 0.95 to 1.07.

Another embodiment comprises cyclosporin A, Surfonic POA-17R2®, and an oil having a specific gravity from 0.95 to 1.07.

Another embodiment comprises cyclosporin A, Surfonic POA-25R2®, and an oil having a specific gravity from 0.95 to 1.07.

Another embodiment comprises cyclosporin A, Surfonic TDA-11®, and an oil having a specific gravity from 0.95 to 1.07.

Another embodiment comprises cyclosporin A, Surfonic TDA-3B®, and an oil having a specific gravity from 0.95 to 1.07.

Another embodiment comprises cyclosporin A, Surfonic TDA-6®, and an oil having a specific gravity from 0.95 to 1.07.

Another embodiment comprises cyclosporin A, Surfonic TDA-6/88®, and an oil having a specific gravity from 0.95 to 1.07.

Another embodiment comprises cyclosporin A, Surfonic TDA-8®, and an oil having a specific gravity from 0.95 to 1.07.

Another embodiment comprises cyclosporin A, Surfonic TDA-8/90®, and an oil having a specific gravity from 0.95 to 1.07.

Another embodiment comprises cyclosporin A, Surfonic TDA-9®, and an oil having a specific gravity from 0.95 to 1.07.

Another embodiment comprises cyclosporin A, Surfonic TX-CCN®, and an oil having a specific gravity from 0.95 to 1.07.

Another embodiment comprises cyclosporin A, Tagat CH 40®, and an oil having a specific gravity from 0.95 to 1.07.

Another embodiment comprises cyclosporin A, Tagat CH 60®, and an oil having a specific gravity from 0.95 to 1.07.

Another embodiment comprises cyclosporin A, Tagat L 2®, and an oil having a specific gravity from 0.95 to 1.07.

Another embodiment comprises cyclosporin A, Tagat S®, and an oil having a specific gravity from 0.95 to 1.07.

Another embodiment comprises cyclosporin A, Tagat S 2®, and an oil having a specific gravity from 0.95 to 1.07.

Another embodiment comprises cyclosporin A, Tally 100 Plus®, and an oil having a specific gravity from 0.95 to 1.07.

Another embodiment comprises cyclosporin A, T-Det A106®, and an oil having a specific gravity from 0.95 to 1.07.

Another embodiment comprises cyclosporin A, T-Det A109®, and an oil having a specific gravity from 0.95 to 1.07.

Another embodiment comprises cyclosporin A, T-Det A136®, and an oil having a specific gravity from 0.95 to 1.07.

Another embodiment comprises cyclosporin A, T-Det A139®, and an oil having a specific gravity from 0.95 to 1.07.

Another embodiment comprises cyclosporin A, T-Det A2412®, and an oil having a specific gravity from 0.95 to 1.07.

Another embodiment comprises cyclosporin A, T-Det A243®, and an oil having a specific gravity from 0.95 to 1.07.

Another embodiment comprises cyclosporin A, T-Det A247®, and an oil having a specific gravity from 0.95 to 1.07.

Another embodiment comprises cyclosporin A, T-Det A249®, and an oil having a specific gravity from 0.95 to 1.07.

Another embodiment comprises cyclosporin A, T-Det A467®, and an oil having a specific gravity from 0.95 to 1.07.

Another embodiment comprises cyclosporin A, T-Det COE®, and an oil having a specific gravity from 0.95 to 1.07.

Another embodiment comprises cyclosporin A, T-Det DD-10®, and an oil having a specific gravity from 0.95 to 1.07.

Another embodiment comprises cyclosporin A, T-Det DD-5®, and an oil having a specific gravity from 0.95 to 1.07.

Another embodiment comprises cyclosporin A, T-Det DD-7®, and an oil having a specific gravity from 0.95 to 1.07.

Another embodiment comprises cyclosporin A, T-Det EPO-104®, and an oil having a specific gravity from 0.95 to 1.07.

Another embodiment comprises cyclosporin A, T-Det EPO-61®, and an oil having a specific gravity from 0.95 to 1.07.

Another embodiment comprises cyclosporin A, T-Det EPO-62®, and an oil having a specific gravity from 0.95 to 1.07.

Another embodiment comprises cyclosporin A, T-Det EPO-64®, and an oil having a specific gravity from 0.95 to 1.07.

Another embodiment comprises cyclosporin A, T-Det N-1.5®, and an oil having a specific gravity from 0.95 to 1.07.

Another embodiment comprises cyclosporin A, T-Det N-10.5®, and an oil having a specific gravity from 0.95 to 1.07.

Another embodiment comprises cyclosporin A, T-Det N-100®, and an oil having a specific gravity from 0.95 to 1.07.

Another embodiment comprises cyclosporin A, T-Det N-1007®, and an oil having a specific gravity from 0.95 to 1.07.

Another embodiment comprises cyclosporin A, T-Det N-12®, and an oil having a specific gravity from 0.95 to 1.07.

Another embodiment comprises cyclosporin A, T-Det N-14®, and an oil having a specific gravity from 0.95 to 1.07.

Another embodiment comprises cyclosporin A, T-Det N-20®, and an oil having a specific gravity from 0.95 to 1.07.

Another embodiment comprises cyclosporin A, T-Det N-30®, and an oil having a specific gravity from 0.95 to 1.07.

Another embodiment comprises cyclosporin A, T-Det N-307®, and an oil having a specific gravity from 0.95 to 1.07.

Another embodiment comprises cyclosporin A, T-Det N-4®, and an oil having a specific gravity from 0.95 to 1.07.

Another embodiment comprises cyclosporin A, T-Det N-40®, and an oil having a specific gravity from 0.95 to 1.07.

Another embodiment comprises cyclosporin A, T-Det N-407®, and an oil having a specific gravity from 0.95 to 1.07.

Another embodiment comprises cyclosporin A, T-Det N-50®, and an oil having a specific gravity from 0.95 to 1.07.

Another embodiment comprises cyclosporin A, T-Det N-507®, and an oil having a specific gravity from 0.95 to 1.07.

Another embodiment comprises cyclosporin A, T-Det N-6®, and an oil having a specific gravity from 0.95 to 1.07.

Another embodiment comprises cyclosporin A, T-Det N-70®, and an oil having a specific gravity from 0.95 to 1.07.

Another embodiment comprises cyclosporin A, T-Det N-8®, and an oil having a specific gravity from 0.95 to 1.07.

Another embodiment comprises cyclosporin A, T-Det N-9.5®, and an oil having a specific gravity from 0.95 to 1.07.

Another embodiment comprises cyclosporin A, T-Det O-12®, and an oil having a specific gravity from 0.95 to 1.07.

Another embodiment comprises cyclosporin A, T-Det O-165®, and an oil having a specific gravity from 0.95 to 1.07.

Another embodiment comprises cyclosporin A, T-Det O-307®, and an oil having a specific gravity from 0.95 to 1.07.

Another embodiment comprises cyclosporin A, T-Det O-40®, and an oil having a specific gravity from 0.95 to 1.07.

Another embodiment comprises cyclosporin A, T-Det O-407®, and an oil having a specific gravity from 0.95 to 1.07.

Another embodiment comprises cyclosporin A, T-Det O-6®, and an oil having a specific gravity from 0.95 to 1.07.

Another embodiment comprises cyclosporin A, T-Det O-8®, and an oil having a specific gravity from 0.95 to 1.07.

Another embodiment comprises cyclosporin A, T-Det O-9®, and an oil having a specific gravity from 0.95 to 1.07.

Another embodiment comprises cyclosporin A, Tegin®, and an oil having a specific gravity from 0.95 to 1.07.

Another embodiment comprises cyclosporin A, Tergitol 15-S-12®, and an oil having a specific gravity from 0.95 to 1.07.

Another embodiment comprises cyclosporin A, Tergitol 15-S-15®, and an oil having a specific gravity from 0.95 to 1.07.

Another embodiment comprises cyclosporin A, Tergitol 15-S-20®, and an oil having a specific gravity from 0.95 to 1.07.

Another embodiment comprises cyclosporin A, Tergitol 15-S-20 (80AQ)®, and an oil having a specific gravity from 0.95 to 1.07.

Another embodiment comprises cyclosporin A, Tergitol 15-S-3®, and an oil having a specific gravity from 0.95 to 1.07.

Another embodiment comprises cyclosporin A, Tergitol 15-S-30®, and an oil having a specific gravity from 0.95 to 1.07.

Another embodiment comprises cyclosporin A, Tergitol 15-S-40®, and an oil having a specific gravity from 0.95 to 1.07.

Another embodiment comprises cyclosporin A, Tergitol 15-S-40 (70AQ)®, and an oil having a specific gravity from 0.95 to 1.07.

Another embodiment comprises cyclosporin A, Tergitol 15-S-5®, and an oil having a specific gravity from 0.95 to 1.07.

Another embodiment comprises cyclosporin A, Tergitol 15-S-7®, and an oil having a specific gravity from 0.95 to 1.07.

Another embodiment comprises cyclosporin A, Tergitol 15-S-9®, and an oil having a specific gravity from 0.95 to 1.07.

Another embodiment comprises cyclosporin A, Tergitol L-101®, and an oil having a specific gravity from 0.95 to 1.07.

Another embodiment comprises cyclosporin A, Tergitol L-61®, and an oil having a specific gravity from 0.95 to 1.07.

Another embodiment comprises cyclosporin A, Tergitol L-62®, and an oil having a specific gravity from 0.95 to 1.07.

Another embodiment comprises cyclosporin A, Tergitol L-64®, and an oil having a specific gravity from 0.95 to 1.07.

Another embodiment comprises cyclosporin A, Tergitol L-81®, and an oil having a specific gravity from 0.95 to 1.07.

Another embodiment comprises cyclosporin A, Tergitol NP-10®, and an oil having a specific gravity from 0.95 to 1.07.

Another embodiment comprises cyclosporin A, Tergitol NP-11®, and an oil having a specific gravity from 0.95 to 1.07.

Another embodiment comprises cyclosporin A, Tergitol NP-12®, and an oil having a specific gravity from 0.95 to 1.07.

Another embodiment comprises cyclosporin A, Tergitol NP-13®, and an oil having a specific gravity from 0.95 to 1.07.

Another embodiment comprises cyclosporin A, Tergitol NP-15®, and an oil having a specific gravity from 0.95 to 1.07.

Another embodiment comprises cyclosporin A, Tergitol NP-30®, and an oil having a specific gravity from 0.95 to 1.07.

Another embodiment comprises cyclosporin A, Tergitol NP-30 (70 AQ)®, and an oil having a specific gravity from 0.95 to 1.07.

Another embodiment comprises cyclosporin A, Tergitol NP-4®, and an oil having a specific gravity from 0.95 to 1.07.

Another embodiment comprises cyclosporin A, Tergitol NP-40®, and an oil having a specific gravity from 0.95 to 1.07.

Another embodiment comprises cyclosporin A, Tergitol NP-40 (70 AQ)®, and an oil having a specific gravity from 0.95 to 1.07.

Another embodiment comprises cyclosporin A, Tergitol NP-50 (70 AQ)®, and an oil having a specific gravity from 0.95 to 1.07.

Another embodiment comprises cyclosporin A, Tergitol NP-55 (70 AQ)®, and an oil having a specific gravity from 0.95 to 1.07.

Another embodiment comprises cyclosporin A, Tergitol NP-6®, and an oil having a specific gravity from 0.95 to 1.07.

Another embodiment comprises cyclosporin A, Tergitol NP-7®, and an oil having a specific gravity from 0.95 to 1.07.

Another embodiment comprises cyclosporin A, Tergitol NP-70 (70 AQ)®, and an oil having a specific gravity from 0.95 to 1.07.

Another embodiment comprises cyclosporin A, Tergitol NP-8®, and an oil having a specific gravity from 0.95 to 1.07.

Another embodiment comprises cyclosporin A, Tergitol NP-9®, and an oil having a specific gravity from 0.95 to 1.07.

Another embodiment comprises cyclosporin A, Tergitol NP-9.5®, and an oil having a specific gravity from 0.95 to 1.07.

Another embodiment comprises cyclosporin A, Tergitol TMN-10®, and an oil having a specific gravity from 0.95 to 1.07.

Another embodiment comprises cyclosporin A, Tergitol TMN-3®, and an oil having a specific gravity from 0.95 to 1.07.

Another embodiment comprises cyclosporin A, Tergitol TMN-6®, and an oil having a specific gravity from 0.95 to 1.07.

Another embodiment comprises cyclosporin A, Tergitol XD®, and an oil having a specific gravity from 0.95 to 1.07.

Another embodiment comprises cyclosporin A, Tergitol XH®, and an oil having a specific gravity from 0.95 to 1.07.

Another embodiment comprises cyclosporin A, Tergitol XJ®, and an oil having a specific gravity from 0.95 to 1.07.

Another embodiment comprises cyclosporin A, Tetronic 1107®, and an oil having a specific gravity from 0.95 to 1.07.

Another embodiment comprises cyclosporin A, Tetronic 1307®, and an oil having a specific gravity from 0.95 to 1.07.

Another embodiment comprises cyclosporin A, Tetronic 150R1®, and an oil having a specific gravity from 0.95 to 1.07.

Another embodiment comprises cyclosporin A, Tetronic 304®, and an oil having a specific gravity from 0.95 to 1.07.

Another embodiment comprises cyclosporin A, Tetronic 701®, and an oil having a specific gravity from 0.95 to 1.07.

Another embodiment comprises cyclosporin A, Tetronic 901®, and an oil having a specific gravity from 0.95 to 1.07.

Another embodiment comprises cyclosporin A, Tetronic 904®, and an oil having a specific gravity from 0.95 to 1.07.

Another embodiment comprises cyclosporin A, Tetronic 908®, and an oil having a specific gravity from 0.95 to 1.07.

Another embodiment comprises cyclosporin A, Tetronic 90R4®, and an oil having a specific gravity from 0.95 to 1.07.

Another embodiment comprises cyclosporin A, Tomadol 1-3®, and an oil having a specific gravity from 0.95 to 1.07.

Another embodiment comprises cyclosporin A, Tomadol 1-5®, and an oil having a specific gravity from 0.95 to 1.07.

Another embodiment comprises cyclosporin A, Tomadol 1-7®, and an oil having a specific gravity from 0.95 to 1.07.

Another embodiment comprises cyclosporin A, Tomadol 23-1®, and an oil having a specific gravity from 0.95 to 1.07.

Another embodiment comprises cyclosporin A, Tomadol 23-3®, and an oil having a specific gravity from 0.95 to 1.07.

Another embodiment comprises cyclosporin A, Tomadol 23-5®, and an oil having a specific gravity from 0.95 to 1.07.

Another embodiment comprises cyclosporin A, Tomadol 23-6.5®, and an oil having a specific gravity from 0.95 to 1.07.

Another embodiment comprises cyclosporin A, Tomadol 25-12®, and an oil having a specific gravity from 0.95 to 1.07.

Another embodiment comprises cyclosporin A, Tomadol 25-3®, and an oil having a specific gravity from 0.95 to 1.07.

Another embodiment comprises cyclosporin A, Tomadol 25-7®, and an oil having a specific gravity from 0.95 to 1.07.

Another embodiment comprises cyclosporin A, Tomadol 25-9®, and an oil having a specific gravity from 0.95 to 1.07.

Another embodiment comprises cyclosporin A, Tomadol 45-13®, and an oil having a specific gravity from 0.95 to 1.07.

Another embodiment comprises cyclosporin A, Tomadol 45-2.5®, and an oil having a specific gravity from 0.95 to 1.07.

Another embodiment comprises cyclosporin A, Tomadol 45-7®, and an oil having a specific gravity from 0.95 to 1.07.

Another embodiment comprises cyclosporin A, Tomadol 91-2.5®, and an oil having a specific gravity from 0.95 to 1.07.

Another embodiment comprises cyclosporin A, Tomadol 91-6®, and an oil having a specific gravity from 0.95 to 1.07.

Another embodiment comprises cyclosporin A, Tomadol 91-8®, and an oil having a specific gravity from 0.95 to 1.07.

Another embodiment comprises cyclosporin A, Triton X-100®, and an oil having a specific gravity from 0.95 to 1.07.

Another embodiment comprises cyclosporin A, Triton X-102®, and an oil having a specific gravity from 0.95 to 1.07.

Another embodiment comprises cyclosporin A, Triton X-114®, and an oil having a specific gravity from 0.95 to 1.07.

Another embodiment comprises cyclosporin A, Triton X-15®, and an oil having a specific gravity from 0.95 to 1.07.

Another embodiment comprises cyclosporin A, Triton X-165 (70%)®, and an oil having a specific gravity from 0.95 to 1.07.

Another embodiment comprises cyclosporin A, Triton X-207®, and an oil having a specific gravity from 0.95 to 1.07.

Another embodiment comprises cyclosporin A, Triton X-305 (70%)®, and an oil having a specific gravity from 0.95 to 1.07.

Another embodiment comprises cyclosporin A, Triton X-35®, and an oil having a specific gravity from 0.95 to 1.07.

Another embodiment comprises cyclosporin A, Triton X-405 (70%)®, and an oil having a specific gravity from 0.95 to 1.07.

Another embodiment comprises cyclosporin A, Triton X-45®, and an oil having a specific gravity from 0.95 to 1.07.

Another embodiment comprises cyclosporin A, Triton X-705 (70%)®, and an oil having a specific gravity from 0.95 to 1.07.

Another embodiment comprises cyclosporin A, Volpo S-10®, and an oil having a specific gravity from 0.95 to 1.07.

Another embodiment comprises cyclosporin A, Volpo S-2®, and an oil having a specific gravity from 0.95 to 1.07.

Another embodiment comprises cyclosporin A, Volpo S-20®, and an oil having a specific gravity from 0.95 to 1.07.

Another embodiment comprises cyclosporin A, Volpo-10®, and an oil having a specific gravity from 0.95 to 1.07.

Another embodiment comprises cyclosporin A, Volpo-20®, and an oil having a specific gravity from 0.95 to 1.07.

Another embodiment comprises cyclosporin A, Volpo-3®, and an oil having a specific gravity from 0.95 to 1.07.

Another embodiment comprises cyclosporin A, Volpo-5®, and an oil having a specific gravity from 0.95 to 1.07.

Another embodiment comprises cyclosporin A, Abex 2515®, and anise oil.

Another embodiment comprises cyclosporin A, Abex 2525/40®, and anise oil.

Another embodiment comprises cyclosporin A, Abex 2535®, and anise oil.

Another embodiment comprises cyclosporin A, Abex 2545®, and anise oil.

Another embodiment comprises cyclosporin A, Acconon E®, and anise oil.

Another embodiment comprises cyclosporin A, Alfonic 1216CO-9®, and anise oil.

Another embodiment comprises cyclosporin A, Alfonic 1412-3®, and anise oil.

Another embodiment comprises cyclosporin A, Alfonic TDA-12®, and anise oil.

Another embodiment comprises cyclosporin A, Alfonic TDA-3®, and anise oil.

Another embodiment comprises cyclosporin A, Alfonic TDA-4®, and anise oil.

Another embodiment comprises cyclosporin A, Alfonic TDA-6®, and anise oil.

Another embodiment comprises cyclosporin A, Alfonic TDA-7®, and anise oil.

Another embodiment comprises cyclosporin A, Alfonic TDA-8®, and anise oil.

Another embodiment comprises cyclosporin A, Alfonic TDA-9®, and anise oil.

Another embodiment comprises cyclosporin A, Alkamuls 400-DO®, and anise oil.

Another embodiment comprises cyclosporin A, Alkamuls 400-MO/E®, and anise oil.

Another embodiment comprises cyclosporin A, Ameroxol OE-10®, and anise oil.

Another embodiment comprises cyclosporin A, Ameroxol OE-2®, and anise oil.

Another embodiment comprises cyclosporin A, Ameroxol OE-20®, and anise oil.

Another embodiment comprises cyclosporin A, Armix 176®, and anise oil.

Another embodiment comprises cyclosporin A, Armix 180-C®, and anise oil.

Another embodiment comprises cyclosporin A, Armix 183®, and anise oil.

Another embodiment comprises cyclosporin A, Brij 35®, and anise oil.

Another embodiment comprises cyclosporin A, Brij 35 Liquid®, and anise oil.

Another embodiment comprises cyclosporin A, Brij 35 SP®, and anise oil.

Another embodiment comprises cyclosporin A, Canasol MJ 2109®, and anise oil.

Another embodiment comprises cyclosporin A, Canasol MJ 52®, and anise oil.

Another embodiment comprises cyclosporin A, Canasol R 3600®, and anise oil.

Another embodiment comprises cyclosporin A, Canasol R 3603®, and anise oil.

Another embodiment comprises cyclosporin A, Canasol R 4000 He, and anise oil.

Another embodiment comprises cyclosporin A, Cedepal CA-210®, and anise oil.

Another embodiment comprises cyclosporin A, Cedepal CA-520®, and anise oil.

Another embodiment comprises cyclosporin A, Cedepal CA-630®, and anise oil.

Another embodiment comprises cyclosporin A, Cedepal CA-720®, and anise oil.

Another embodiment comprises cyclosporin A, Cedepal CA-890®, and anise oil.

Another embodiment comprises cyclosporin A, Cedepal CA-897®, and anise oil.

Another embodiment comprises cyclosporin A, Cedepal CO-210®, and anise oil.

Another embodiment comprises cyclosporin A, Cedepal CO-430®, and anise oil.

Another embodiment comprises cyclosporin A, Cedepal CO-530®, and anise oil.

Another embodiment comprises cyclosporin A, Cedepal CO-630®, and anise oil.

Another embodiment comprises cyclosporin A, Cedepal CO-710®, and anise oil.

Another embodiment comprises cyclosporin A, Cedepal CO-730®, and anise oil.

Another embodiment comprises cyclosporin A, Cedepal CO-880®, and anise oil.

Another embodiment comprises cyclosporin A, Cedepal CO-887®, and anise oil.

Another embodiment comprises cyclosporin A, Cedepal CO-890®, and anise oil.

Another embodiment comprises cyclosporin A, Cedepal CO-897®, and anise oil.

Another embodiment comprises cyclosporin A, Cedepal CO-970®, and anise oil.

Another embodiment comprises cyclosporin A, Cedepal CO-977®, and anise oil.

Another embodiment comprises cyclosporin A, Cedepal CO-990®, and anise oil.

Another embodiment comprises cyclosporin A, Cedepal CO-997®, and anise oil.

Another embodiment comprises cyclosporin A, Chemal BP 261®, and anise oil.

Another embodiment comprises cyclosporin A, Chemal BP 262®, and anise oil.

Another embodiment comprises cyclosporin A, Chemal BP 3172®, and anise oil.

Another embodiment comprises cyclosporin A, Chemal BP 3174®, and anise oil.

Another embodiment comprises cyclosporin A, Chemal DA-4®, and anise oil.

Another embodiment comprises cyclosporin A, Chemal DA-6®, and anise oil.

Another embodiment comprises cyclosporin A, Chemal DA-9®, and anise oil.

Another embodiment comprises cyclosporin A, Chemal LA-23®, and anise oil.

Another embodiment comprises cyclosporin A, Chemal LA-4®, and anise oil.

Another embodiment comprises cyclosporin A, Chemal LA-9®, and anise oil.

Another embodiment comprises cyclosporin A, Chemal OA-20G®, and anise oil.

Another embodiment comprises cyclosporin A, Chemal OA-5®, and anise oil.

Another embodiment comprises cyclosporin A, Chemal OA-9®, and anise oil.

Another embodiment comprises cyclosporin A, Chemal TDA-12®, and anise oil.

Another embodiment comprises cyclosporin A, Chemal TDA-15®, and anise oil.

Another embodiment comprises cyclosporin A, Chemal TDA-3®, and anise oil.

Another embodiment comprises cyclosporin A, Chemal TDA-6®, and anise oil.

Another embodiment comprises cyclosporin A, Chemal TDA-9®, and anise oil.

Another embodiment comprises cyclosporin A, Chemax CO-16®, and anise oil.

Another embodiment comprises cyclosporin A, Chemax CO-200/50®, and anise oil.

Another embodiment comprises cyclosporin A, Chemax CO-25®, and anise oil.

Another embodiment comprises cyclosporin A, Chemax CO-30®, and anise oil.

Another embodiment comprises cyclosporin A, Chemax CO-36®, and anise oil.

Another embodiment comprises cyclosporin A, Chemax CO-40®, and anise oil.

Another embodiment comprises cyclosporin A, Chemax CO-5®, and anise oil.

Another embodiment comprises cyclosporin A, Chemax CO-80®, and anise oil.

Another embodiment comprises cyclosporin A, Chemax DNP-150/50®, and-anise oil.

Another embodiment comprises cyclosporin A, Chemax DNP-18®, and anise oil.

Another embodiment comprises cyclosporin A, Chemax DNP-8®, and anise oil.

Another embodiment comprises cyclosporin A, Chemax E 200 ML®, and anise oil.

Another embodiment comprises cyclosporin A, Chemax E 200 MO®, and anise oil.

Another embodiment comprises cyclosporin A, Chemax E 400 ML®, and anise oil.

Another embodiment comprises cyclosporin A, Chemax E 400 MO®, and anise oil.

Another embodiment comprises cyclosporin A, Chemax E 600 ML®, and anise oil.

Another embodiment comprises cyclosporin A, Chemax E 600 MO®, and anise oil.

Another embodiment comprises cyclosporin A, Chemax E1000 MO®, and anise oil.

Another embodiment comprises cyclosporin A, Chemax E1000 MS®, and anise oil.

Another embodiment comprises cyclosporin A, Chemax E200 MS®, and anise oil.

Another embodiment comprises cyclosporin A, Chemax E400 MS®, and anise oil.

Another embodiment comprises cyclosporin A, Chemax E600 MS®, and anise oil.

Another embodiment comprises cyclosporin A, Chemax HCO-16®, and anise oil.

Another embodiment comprises cyclosporin A, Chemax HCO-200/50®, and anise oil.

Another embodiment comprises cyclosporin A, Chemax HCO-25®, and anise oil.

Another embodiment comprises cyclosporin A, Chemax HCO-5®, and anise oil.

Another embodiment comprises cyclosporin A, Chemax TO-16®, and anise oil.

Another embodiment comprises cyclosporin A, Chemax TO-8®, and anise oil.

Another embodiment comprises cyclosporin A, Colalube 3409®, and anise oil.

Another embodiment comprises cyclosporin A, Colalube 3411®, and anise oil.

Another embodiment comprises cyclosporin A, Colalube 3413®, and anise oil.

Another embodiment comprises cyclosporin A, Colalube 3414®, and anise oil.

Another embodiment comprises cyclosporin A, Colalube 3415®, and anise oil.

Another embodiment comprises cyclosporin A, Colalube 3416®, and anise oil.

Another embodiment comprises cyclosporin A, Colalube 3417®, and anise oil.

Another embodiment comprises cyclosporin A, Delonic NPE-100®, and anise oil.

Another embodiment comprises cyclosporin A, Delonic NPE-30®, and anise oil.

Another embodiment comprises cyclosporin A, Delonic NPE-40®, and anise oil.

Another embodiment comprises cyclosporin A, Delonic OPE-10®, and anise oil.

Another embodiment comprises cyclosporin A, Delonic OPE-12®, and anise oil.

Another embodiment comprises cyclosporin A, Delonic OPE-30®, and anise oil.

Another embodiment comprises cyclosporin A, Delonic OPE-40®, and anise oil.

Another embodiment comprises cyclosporin A, Delonic OPE-5®, and anise oil.

Another embodiment comprises cyclosporin A, Delonic OPE-7.5®, and anise oil.

Another embodiment comprises cyclosporin A, DePeg 16-CO®, and anise oil.

Another embodiment comprises cyclosporin A, DePeg 25-CO®, and anise oil.

Another embodiment comprises cyclosporin A, DePeg 30-CO®, and anise oil.

Another embodiment comprises cyclosporin A, DePeg 40-CO®, and anise oil.

Another embodiment comprises cyclosporin A, DePeg 5-CO®, and anise oil.

Another embodiment comprises cyclosporin A, DePeg 80-CO®, and anise oil.

Another embodiment comprises cyclosporin A, DeSonic S-100®, and anise oil.

Another embodiment comprises cyclosporin A, DeSonic S-114®, and anise oil.

Another embodiment comprises cyclosporin A, DeSonic S-405®, and anise oil.

Another embodiment comprises cyclosporin A, DeSonic S-45®, and anise oil.

Another embodiment comprises cyclosporin A, DeSonic TDA-9®, and anise oil.

Another embodiment comprises cyclosporin A, DeThox Acid O-14®, and anise oil.

Another embodiment comprises cyclosporin A, DeThox Acid O-16®, and anise oil.

Another embodiment comprises cyclosporin A, DeThox Acid O-9®, and anise oil.

Another embodiment comprises cyclosporin A, DeThox Acid TO-14®, and anise oil.

Another embodiment comprises cyclosporin A, DeThox Acid TO-16.5®, and anise oil.

Another embodiment comprises cyclosporin A, DeThox Acid TO-8.5®, and anise oil.

Another embodiment comprises cyclospbrin A, Dur-Em 114®, and anise oil.

Another embodiment comprises cyclosporin A, Dur-Em 117®, and anise oil.

Another embodiment comprises cyclosporin A, Dur-Em 204®, and anise oil.

Another embodiment comprises cyclosporin A, Dur-Em 207®, and anise oil.

Another embodiment comprises cyclosporin A, Dur-Em 207E®, and anise oil.

Another embodiment comprises cyclosporin A, Emerest 2704®, and anise oil.

Another embodiment comprises cyclosporin A, Emerest 2712®, and anise oil.

Another embodiment comprises cyclosporin A, Emerest 2715®, and anise oil.

Another embodiment comprises cyclosporin A, Ethal EH-2®, and anise oil.

Another embodiment comprises cyclosporin A, Ethal EH-5®, and anise oil.

Another embodiment comprises cyclosporin A, Ethal OA-23®, and anise oil.

Another embodiment comprises cyclosporin A, Ethosperse CA-20®, and anise oil.

Another embodiment comprises cyclosporin A, Ethosperse LA-12®, and anise oil.

Another embodiment comprises cyclosporin A, Ethosperse LA-23®, and anise oil.

Another embodiment comprises cyclosporin A, Ethosperse LA-4®, and anise oil.

Another embodiment comprises cyclosporin A, Ethox 3095®, and anise oil.

Another embodiment comprises cyclosporin A, Ethox CO-16®, and anise oil.

Another embodiment comprises cyclosporin A, Ethox CO-200®, and anise oil.

Another embodiment comprises cyclosporin A, Ethox CO-25®, and anise oil.

Another embodiment comprises cyclosporin A, Ethox CO-30®, and anise oil.

Another embodiment comprises cyclosporin A, Ethox CO-36®, and anise oil.

Another embodiment comprises cyclosporin A, Ethox CO-40®, and anise oil.

Another embodiment comprises cyclosporin A, Ethox CO-5®, and anise oil.

Another embodiment comprises cyclosporin A, Ethox DO-14®, and anise oil.

Another embodiment comprises cyclosporin A, Ethox DO-9®, and anise oil.

Another embodiment comprises cyclosporin A, Ethox MA-8®, and anise oil.

Another embodiment comprises cyclosporin A, Ethox MI-14i, and anise oil.

Another embodiment comprises cyclosporin A, Ethox ML-14®, and anise oil.

Another embodiment comprises cyclosporin A, Ethox ML-5®, and anise oil.

Another embodiment comprises cyclosporin A, Ethox MO-14®, and anise oil.

Another embodiment comprises cyclosporin A, Ethox MO-9®, and anise oil.

Another embodiment comprises cyclosporin A, Ethox MS-14®, and anise oil.

Another embodiment comprises cyclosporin A, Ethox MS-23®, and anise oil.

Another embodiment comprises cyclosporin A, Ethox MS-40®, and anise oil.

Another embodiment comprises cyclosporin A, Ethox MS-8®, and anise oil.

Another embodiment comprises cyclosporin A, Ethox TO-16®, and anise oil.

Another embodiment comprises cyclosporin A, Ethox TO-8®, and anise oil.

Another embodiment comprises cyclosporin A, Ethylan SN-120®, and anise oil.

Another embodiment comprises cyclosporin A, Ethylan SN-70®, and anise oil.

Another embodiment comprises cyclosporin A, Ethylan SN-90®, and anise oil.

Another embodiment comprises cyclosporin A, Flexricin 13®, and anise oil.

Another embodiment comprises cyclosporin A, Flexricin 15®, and anise oil.

Another embodiment comprises cyclosporin A, Genapol 1261®, and anise oil.

Another embodiment comprises cyclosporin A, Generol 122 E25®, and anise oil.

Another embodiment comprises cyclosporin A, Glicepol 160®, and anise oil.

Another embodiment comprises cyclosporin A, Glicepol 560®, and anise oil.

Another embodiment comprises cyclosporin A, Glicepol GMS 20®, and anise oil.

Another embodiment comprises cyclosporin A, Hetoxide BN-13®, and anise oil.

Another embodiment comprises cyclosporin A, Hetoxide DNP-4®, and anise oil.

Another embodiment comprises cyclosporin A, Hetoxide DNP-9.6®, and anise oil.

Another embodiment comprises cyclosporin A, Hetoxide G-26®, and anise oil.

Another embodiment comprises cyclosporin A, Hetoxide G-7®, and anise oil.

Another embodiment comprises cyclosporin A, Hetoxide GT-20®, and anise oil.

Another embodiment comprises cyclosporin A, Hetoxide HC-40®, and anise oil.

Another embodiment comprises cyclosporin A, Hetoxol C-24®, and anise oil.

Another embodiment comprises cyclosporin A, Hetoxol CA-10®, and anise oil.

Another embodiment comprises cyclosporin A, Hetoxol CA-2®, and anise oil.

Another embodiment comprises cyclosporin A, Hetoxol CA-20®, and anise oil.

Another embodiment comprises cyclosporin A, Hetoxol CAWS®, and anise oil.

Another embodiment comprises cyclosporin A, Hetoxol CD-3®, and anise oil.

Another embodiment comprises cyclosporin A, Hetoxol CD-4®, and anise oil.

Another embodiment comprises cyclosporin A, Hetoxol CS-15®, and anise oil.

Another embodiment comprises cyclosporin A, Hetoxol CS-20®, and anise oil.

Another embodiment comprises cyclosporin A, Hetoxol CS-20D®, and anise oil.

Another embodiment comprises cyclosporin A, Hetoxol CS-25®, and anise oil.

Another embodiment comprises cyclosporin A, Hetoxol CS-50®, and anise oil.

Another embodiment comprises cyclosporin A, Hetoxol CS-9®, and anise oil.

Another embodiment comprises cyclosporin A, Hetoxol CSA-15®, and anise oil.

Another embodiment comprises cyclosporin A, Hetoxol D®, and anise oil.

Another embodiment comprises cyclosporin A, Hetoxol G®, and anise oil.

Another embodiment comprises cyclosporin A, Hetoxol J®, and anise oil.

Another embodiment comprises cyclosporin A, Hetoxol L®, and anise oil.

Another embodiment comprises cyclosporin A, Hetoxol L-23®, and anise oil.

Another embodiment comprises cyclosporin A, Hetoxol L-4®, and anise oil.

Another embodiment comprises cyclosporin A, Hetoxol L-9®, and anise oil.

Another embodiment comprises cyclosporin A, Hetoxol LS-9®, and anise oil.

Another embodiment comprises cyclosporin A, Hetoxol M-3®, and anise oil.

Another embodiment comprises cyclosporin A, Hetoxol OA-10 Special®, and anise oil.

Another embodiment comprises cyclosporin A, Hetoxol OA-20 Special®, and anise oil.

Another embodiment comprises cyclosporin A, Hetoxol OA-3 Special®, and anise oil.

Another embodiment comprises cyclosporin A, Hetoxol OA-5 Special®, and anise oil.

Another embodiment comprises cyclosporin A, Hetoxol OL-10®, and anise oil.

Another embodiment comprises cyclosporin A, Hetoxol OL-2®, and anise oil.

Another embodiment comprises cyclosporin A, Hetoxol OL-23®, and anise oil.

Another embodiment comprises cyclosporin A, Hetoxol OL-4®, and anise oil.

Another embodiment comprises cyclosporin A, Hetoxol OL-40®, and anise oil.

Another embodiment comprises cyclosporin A, Hetoxol OL-5®, and anise oil.

Another embodiment comprises cyclosporin A, Hetoxol PLA®, and anise oil.

Another embodiment comprises cyclosporin A, Hetoxol SP-15®, and anise oil.

Another embodiment comprises cyclosporin A, Hetoxol STA-10®, and anise oil.

Another embodiment comprises cyclosporin A, Hetoxol STA-2®, and anise oil.

Another embodiment comprises cyclosporin A, Hetoxol STA-30®, and anise oil.

Another embodiment comprises cyclosporin A, Hetoxol TD-12®, and anise oil.

Another embodiment comprises cyclosporin A, Hetoxol TD-18®, and anise oil.

Another embodiment comprises cyclosporin A, Hetoxol TD-3®, and anise oil.

Another embodiment comprises cyclosporin A, Hetoxol TD-6®, and anise oil.

Another embodiment comprises cyclosporin A, Hetoxol TD-9®, and anise oil.

Another embodiment comprises cyclosporin A, Hetoxol TDEP-15®, and anise oil.

Another embodiment comprises cyclosporin A, Hetoxol TDEP-63®, and anise oil.

Another embodiment comprises cyclosporin A, Iconol 24-12®, and anise oil.

Another embodiment comprises cyclosporin A, Iconol 24-23®, and anise oil.

Another embodiment comprises cyclosporin A, Iconol 24-4®, and anise oil.

Another embodiment comprises cyclosporin A, Iconol DA-4®, and anise oil.

Another embodiment comprises cyclosporin A, Iconol DA-6®, and anise oil.

Another embodiment comprises cyclosporin A, Iconol TDA-10®, and anise oil.

Another embodiment comprises cyclosporin A, Iconol TDA-3®, and anise oil.

Another embodiment comprises cyclosporin A, Iconol TDA-6®, and anise oil.

Another embodiment comprises cyclosporin A, Iconol TDA-8-90%®, and anise oil.

Another embodiment comprises cyclosporin A, Iconol TDA-9®, and anise oil.

Another embodiment comprises cyclosporin A, Igepal CA-210®, and anise oil.

Another embodiment comprises cyclosporin A, Igepal CA-407®, and anise oil.

Another embodiment comprises cyclosporin A, Igepal CA-420®, and anise oil.

Another embodiment comprises cyclosporin A, Igepal CA-520®, and anise oil.

Another embodiment comprises cyclosporin A, Igepal CA-620®, and anise oil.

Another embodiment comprises cyclosporin A, Igepal CA-630®, and anise oil.

Another embodiment comprises cyclosporin A, Igepal CA-720®, and anise oil.

Another embodiment comprises cyclosporin A, Igepal CA-877®, and anise oil.

Another embodiment comprises cyclosporin A, Igepal CA-887®, and anise oil.

Another embodiment comprises cyclosporin A, Igepal CA-897®, and anise oil.

Another embodiment comprises cyclosporin A, Igepal CO-210®, and anise oil.

Another embodiment comprises cyclosporin A, Igepal CO-430®, and anise oil.

Another embodiment comprises cyclosporin A, Igepal CO-520®, and anise oil.

Another embodiment comprises cyclosporin A, Igepal CO-530®, and anise oil.

Another embodiment comprises cyclosporin A, Igepal CO-610®, and anise oil.

Another embodiment comprises cyclosporin A, Igepal CO-630®, and anise oil.

Another embodiment comprises cyclosporin A, Igepal CO-630 Special®, and anise oil.

Another embodiment comprises cyclosporin A, Igepal CO-660®, and anise oil.

Another embodiment comprises cyclosporin A, Igepal CO-710®, and anise oil.

Another embodiment comprises cyclosporin A, Igepal CO-720®, and anise oil.

Another embodiment comprises cyclosporin A, Igepal CO-730®, and anise oil.

Another embodiment comprises cyclosporin A, Igepal CO-738®, and anise oil.

Another embodiment comprises cyclosporin A, Igepal CO-850®, and anise oil.

Another embodiment comprises cyclosporin A, Igepal CO-880®, and anise oil.

Another embodiment comprises cyclosporin A, Igepal CO-887®, and anise oil.

Another embodiment comprises cyclosporin A, Igepal CO-890®, and anise oil.

Another embodiment comprises cyclosporin A, Igepal CO-897®, and anise oil.

Another embodiment comprises cyclosporin A, Igepal CO-970®, and anise oil.

Another embodiment comprises cyclosporin A, Igepal CO-977®, and anise oil.

Another embodiment comprises cyclosporin A, Igepal CO-987®, and anise oil.

Another embodiment comprises cyclosporin A, Igepal CO-990 FLK®, and anise oil.

Another embodiment comprises cyclosporin A, Igepal CO-997®, and anise oil.

Another embodiment comprises cyclosporin A, Igepal DM-430®, and anise oil.

Another embodiment comprises cyclosporin A, Igepal DM-530®, and anise oil.

Another embodiment comprises cyclosporin A, Igepal DM-710®, and anise oil.

Another embodiment comprises cyclosporin A, Igepal DM-730®, and anise oil.

Another embodiment comprises cyclosporin A, Igepal DM-880®, and anise oil.

Another embodiment comprises cyclosporin A, Igepal DM-970 FLK®, and anise oil.

Another embodiment comprises cyclosporin A, Igepal OD-410®, and anise oil.

Another embodiment comprises cyclosporin A, Igepal RC-520®, and anise oil.

Another embodiment comprises cyclosporin A, Igepal RC-620®, and anise oil.

Another embodiment comprises cyclosporin A, Igepal RC-630®, and anise oil.

Another embodiment comprises cyclosporin A, Lexemul CS-20®, and anise oil.

Another embodiment comprises cyclosporin A, Lipo GMS 450®, and anise oil.

Another embodiment comprises cyclosporin A, Lipo Polyglycol 1000®, and anise oil.

Another embodiment comprises cyclosporin A, Lipo Polyglycol 200®, and anise oil.

Another embodiment comprises cyclosporin A, Lipo Polyglycol 300®, and anise oil.

Another embodiment comprises cyclosporin A, Lipo Polyglycol 3350®, and anise oil.

Another embodiment comprises cyclosporin A, Lipo Polyglycol 400®, and anise oil.

Another embodiment comprises cyclosporin A, Lipo Polyglycol 600®, and anise oil.

Another embodiment comprises cyclosporin A, Lipocol C-10®, and anise oil.

Another embodiment comprises cyclosporin A, Lipocol C-2®, and anise oil.

Another embodiment comprises cyclosporin A, Lipocol C-20®, and anise oil.

Another embodiment comprises cyclosporin A, Lipocol L-12®, and anise oil.

Another embodiment comprises cyclosporin A, Lipocol L-23®, and anise oil.

Another embodiment comprises cyclosporin A, Lipocol L-4®, and anise oil.

Another embodiment comprises cyclosporin A, Lipocol O-10®, and anise oil.

Another embodiment comprises cyclosporin A, Lipocol O-2®, and anise oil.

Another embodiment comprises cyclosporin A, Lipocol O-20®, and anise oil.

Another embodiment comprises cyclosporin A, Lipocol O-5®, and anise oil.

Another embodiment comprises cyclosporin A, Lipocol S-10®, and anise oil.

Another embodiment comprises cyclosporin A, Lipocol S-2®, and anise oil.

Another embodiment comprises cyclosporin A, Lipocol S-20®, and anise oil.

Another embodiment comprises cyclosporin A, Lipocol SC-15®, and anise oil.

Another embodiment comprises cyclosporin A, Lipocol SC-20®, and anise oil.

Another embodiment comprises cyclosporin A, Lipocol SC-4®, and anise oil.

Another embodiment comprises cyclosporin A, Lipocol TD-12®, and anise oil.

Another embodiment comprises cyclosporin A, Lumulse 40-O K®, and anise oil.

Another embodiment comprises cyclosporin A, Lumulse 42-O K®, and anise oil.

Another embodiment comprises cyclosporin A, Lumulse GMT-40®, and anise oil.

Another embodiment comprises cyclosporin A, Lumulse PEG 1450®, and anise oil.

Another embodiment comprises cyclosporin A, Lumulse PEG 1450 NF®, and anise oil.

Another embodiment comprises cyclosporin A, Lumulse PEG 8000®, and anise oil.

Another embodiment comprises cyclosporin A, Lumulse PGO®, and anise oil.

Another embodiment comprises cyclosporin A, Lumulse POE (2) Oleyl Amine®, and anise oil.

Another embodiment comprises cyclosporin A, Magrabar PGO-104®, and anise oil.

Another embodiment comprises cyclosporin A, Mapeg 200 ML®, and anise oil.

Another embodiment comprises cyclosporin A, Mapeg 400 MO®, and anise oil.

Another embodiment comprises cyclosporin A, Mapeg 400 MOT®, and anise oil.

Another embodiment comprises cyclosporin A, Mapeg 400DO®, and anise oil.

Another embodiment comprises cyclosporin A, Mapeg 400DOT®, and anise oil.

Another embodiment comprises cyclosporin A, Mapeg 600DOT®, and anise oil.

Another embodiment comprises cyclosporin A, Marlox B 24/80®, and anise oil.

Another embodiment comprises cyclosporin A, Marlox FK 86®, and anise oil.

Another embodiment comprises cyclosporin A, Marlox MO 154®, and anise oil.

Another embodiment comprises cyclosporin A, Mazox CDA CG®, and anise oil.

Another embodiment comprises cyclosporin A, Monoglycerides Glyceryl Monestearate®, and anise oil.

Another embodiment comprises cyclosporin A, Myrj 45®, and anise oil.

Another embodiment comprises cyclosporin A, Myrj 52®, and anise oil.

Another embodiment comprises cyclosporin A, Myrj 52S®, and anise oil.

Another embodiment comprises cyclosporin A, Myrj 53®, and anise oil.

Another embodiment comprises cyclosporin A, Myrj 59®, and anise oil.

Another embodiment comprises cyclosporin A, Norfox NP-12®, and anise oil.

Another embodiment comprises cyclosporin A, Norfox NP-4®, and anise oil.

Another embodiment comprises cyclosporin A, Norfox NP-6®, and anise oil.

Another embodiment comprises cyclosporin A, Norfox NP-887®, and anise oil.

Another embodiment comprises cyclosporin A, Norfox NP-9®, and anise oil.

Another embodiment comprises cyclosporin A, Norfox NP-977®, and anise oil.

Another embodiment comprises cyclosporin A, Norfox OP-100®, and anise oil.

Another embodiment comprises cyclosporin A, Norfox OP-102®, and anise oil.

Another embodiment comprises cyclosporin A, Norfox OP-114®, and anise oil.

Another embodiment comprises cyclosporin A, Norfox OP-45®, and anise oil.

Another embodiment comprises cyclosporin A, Pegosperse 100-L®, and anise oil.

Another embodiment comprises cyclosporin A, Pegosperse 100-O®, and anise oil.

Another embodiment comprises cyclosporin A, Pegosperse 100-S®, and anise oil.

Another embodiment comprises cyclosporin A, Pegosperse 1500-MS®, and anise oil.

Another embodiment comprises cyclosporin A, Pegosperse 1750-MS®, and anise oil.

Another embodiment comprises cyclosporin A, Pegosperse 200 DL®, and anise oil.

Another embodiment comprises cyclosporin A, Pegosperse 200-ML®, and anise oil.

Another embodiment comprises cyclosporin A, Pegosperse 400-DL®, and anise oil.

Another embodiment comprises cyclosporin A, Pegosperse 400-DO®, and anise oil.

Another embodiment comprises cyclosporin A, Pegosperse 400-DS®, and anise oil.

Another embodiment comprises cyclosporin A, Pegosperse 400-ML®, and anise oil.

Another embodiment comprises cyclosporin A, Pegosperse 400-MO®, and anise oil.

Another embodiment comprises cyclosporin A, Pegosperse 400-MOT®, and anise oil.

Another embodiment comprises cyclosporin A, Pegosperse 400-MS®, and anise oil.

Another embodiment comprises cyclosporin A, Pegosperse 50-DS®, and anise oil.

Another embodiment comprises cyclosporin A, Pegosperse 50-MS®, and anise oil.

Another embodiment comprises cyclosporin A, Pegosperse 600-ML®, and anise oil.

Another embodiment comprises cyclosporin A, Pegosperse 600-MS®, and anise oil.

Another embodiment comprises cyclosporin A, Plurafac A-38®, and anise oil.

Another embodiment comprises cyclosporin A, Plurafac B-25-5®, and anise oil.

Another embodiment comprises cyclosporin A, Plurafac B-26®, and anise oil.

Another embodiment comprises cyclosporin A, Plurafac D-25®, and anise oil.

Another embodiment comprises cyclosporin A, Plurafac LF 131®, and anise oil.

Another embodiment comprises cyclosporin A, Plurafac LF 4030®, and anise oil.

Another embodiment comprises cyclosporin A, Plurafac LF 7000®, and anise oil.

Another embodiment comprises cyclosporin A, Plurafac RA-20®, and anise oil.

Another embodiment comprises cyclosporin A, Plurafac RA-30®, and anise oil.

Another embodiment comprises cyclosporin A, Plurafac RA-40®, and anise oil.

Another embodiment comprises cyclosporin A, Pluronic 10R5®, and anise oil.

Another embodiment comprises cyclosporin A, Pluronic 17R2®, and anise oil.

Another embodiment comprises cyclosporin A, Pluronic 17R4®, and anise oil.

Another embodiment comprises cyclosporin A, Pluronic 25R2®, and anise oil.

Another embodiment comprises cyclosporin A, Pluronic 25R4®, and anise oil.

Another embodiment comprises cyclosporin A, Pluronic 31R1®, and anise oil.

Another embodiment comprises cyclosporin A, Pluronic F108®, and anise oil.

Another embodiment comprises cyclosporin A, Pluronic F127®, and anise oil.

Another embodiment comprises cyclosporin A, Pluronic F38®, and anise oil.

Another embodiment comprises cyclosporin A, Pluronic F68®, and anise oil.

Another embodiment comprises cyclosporin A, Pluronic F68LF®, and anise oil.

Another embodiment comprises cyclosporin A, Pluronic F77®, and anise oil.

Another embodiment comprises cyclosporin A, Pluronic F87®, and anise oil.

Another embodiment comprises cyclosporin A, Pluronic F88®, and anise oil.

Another embodiment comprises cyclosporin A, Pluronic F98®, and anise oil.

Another embodiment comprises cyclosporin A, Pluronic L10®, and anise oil.

Another embodiment comprises cyclosporin A, Pluronic L101®, and anise oil.

Another embodiment comprises cyclosporin A, Pluronic L121®, and anise oil.

Another embodiment comprises cyclosporin A, Pluronic L31®, and anise oil.

Another embodiment comprises cyclosporin A, Pluronic L35®, and anise oil.

Another embodiment comprises cyclosporin A, Pluronic L43®, and anise oil.

Another embodiment comprises cyclosporin A, Pluronic L44®, and anise oil.

Another embodiment comprises cyclosporin A, Pluronic L61®, and anise oil.

Another embodiment comprises cyclosporin A, Pluronic L62®, and anise oil.

Another embodiment comprises cyclosporin A, Pluronic L62D®, and anise oil.

Another embodiment comprises cyclosporin A, Pluronic L62LF®, and anise oil.

Another embodiment comprises cyclosporin A, Pluronic L64®, and anise oil.

Another embodiment comprises cyclosporin A, Pluronic L81®, and anise oil.

Another embodiment comprises cyclosporin A, Pluronic L92®, and anise oil.

Another embodiment comprises cyclosporin A, Pluronic P103®, and anise oil.

Another embodiment comprises cyclosporin A, Pluronic P104®, and anise oil.

Another embodiment comprises cyclosporin A, Pluronic P105®, and anise oil.

Another embodiment comprises cyclosporin A, Pluronic P123®, and anise oil.

Another embodiment comprises cyclosporin A, Pluronic P65®, and anise oil.

Another embodiment comprises cyclosporin A, Pluronic P84®, and anise oil.

Another embodiment comprises cyclosporin A, Pluronic P85®, and anise oil.

Another embodiment comprises cyclosporin A, Polyaldo 10-2-P®, and anise oil.

Another embodiment comprises cyclosporin A, Polyaldo DGDO®, and anise oil.

Another embodiment comprises cyclosporin A, Polyaldo HGDS®, and anise oil.

Another embodiment comprises cyclosporin A, Polyaldo TGMS®, and anise oil.

Another embodiment comprises cyclosporin A, Protegin V®, and anise oil.

Another embodiment comprises cyclosporin A, Protegin W®, and anise oil.

Another embodiment comprises cyclosporin A, Protegin WX®, and anise oil.

Another embodiment comprises cyclosporin A, Protegin XV®, and anise oil.

Another embodiment comprises cyclosporin A, Rhodasurf BC-420®, and anise oil.

Another embodiment comprises cyclosporin A, Rhodasurf BC-610®, and anise oil.

Another embodiment comprises cyclosporin A, Rhodasurf BC-630®, and anise oil.

Another embodiment comprises cyclosporin A, Rhodasurf BC-720®, and anise oil.

Another embodiment comprises cyclosporin A, Rhodasurf BC-840®, and anise oil.

Another embodiment comprises cyclosporin A, Rhodasurf L 1®, and anise oil.

Another embodiment comprises cyclosporin A, Rhodasurf L 12®, and anise oil.

Another embodiment comprises cyclosporin A, Rhodasurf L 2®, and anise oil.

Another embodiment comprises cyclosporin A, Rhodasurf L 20®, and anise oil.

Another embodiment comprises cyclosporin A, Rhodasurf L 3®, and anise oil.

Another embodiment comprises cyclosporin A, Rhodasurf L 30®, and anise oil.

Another embodiment comprises cyclosporin A, Rhodasurf L 4®, and anise oil.

Another embodiment comprises cyclosporin A, Rhodasurf L 50®, and anise oil.

Another embodiment comprises cyclosporin A, Rhodasurf L 7/90®, and anise oil.

Another embodiment comprises cyclosporin A, Rhodasurf LA-3®, and anise oil.

Another embodiment comprises cyclosporin A, Rhodasurf LA-7®, and anise oil.

Another embodiment comprises cyclosporin A, Rhodasurf LAN-23®, and anise oil.

Another embodiment comprises cyclosporin A, Rhodasurf ON-870®, and anise oil.

Another embodiment comprises cyclosporin A, Rhodasurf ON-877®, and anise oil.

Another embodiment comprises cyclosporin A, Rhodasurf TDA-8/5®, and anise oil.

Another embodiment comprises cyclosporin A, Ritapro 100®, and anise oil.

Another embodiment comprises cyclosporin A, Ritapro 165®, and anise oil.

Another embodiment comprises cyclosporin A, Ritapro 200®, and anise oil.

Another embodiment comprises cyclosporin A, Ritapro 300®, and anise oil.

Another embodiment comprises cyclosporin A, Ritoleth 10®, and anise oil.

Another embodiment comprises cyclosporin A, Ritoleth 2®, and anise oil.

Another embodiment comprises cyclosporin A, Ritoleth 20®, and anise oil.

Another embodiment comprises cyclosporin A, Ritoleth 5®, and anise oil.

Another embodiment comprises cyclosporin A, Santone 10-10-0®, and anise oil.

Another embodiment comprises cyclosporin A, Santone 3-1-S XTR®, and anise oil.

Another embodiment comprises cyclosporin A, Santone 3-1-SH®, and anise oil.

Another embodiment comprises cyclosporin A, Santone 8-1-O®, and anise oil.

Another embodiment comprises cyclosporin A, Solulan 16®, and anise oil.

Another embodiment comprises cyclosporin A, Solulan 75®, and anise oil.

Another embodiment comprises cyclosporin A, Solulan C-24®, and anise oil.

Another embodiment comprises cyclosporin A, Solulan L-575®, and anise oil.

Another embodiment comprises cyclosporin A, Standamox CAW®, and anise oil.

Another embodiment comprises cyclosporin A, Surfine AZI-A®, and anise oil.

Another embodiment comprises cyclosporin A, Surfonic CO-15®, and anise oil.

Another embodiment comprises cyclosporin A, Surfonic CO-25®, and anise oil.

Another embodiment comprises cyclosporin A, Surfonic CO-30®, and anise oil.

Another embodiment comprises cyclosporin A, Surfonic CO-36®, and anise oil.

Another embodiment comprises cyclosporin A, Sunonic CO-42®, and anise oil.

Another embodiment comprises cyclosporin A, Surfonic E400-MO®, and anise oil.

Another embodiment comprises cyclosporin A, Surfonic L12-3®, and anise oil.

Another embodiment comprises cyclosporin A, Surfonic L12-6®, and anise oil.

Another embodiment comprises cyclosporin A, Surfonic L12-8®, and anise oil.

Another embodiment comprises cyclosporin A, Surfonic L24-12®, and anise oil.

Another embodiment comprises cyclosporin A, Surfonic L24-2®, and anise oil.

Another embodiment comprises cyclosporin A, Surfonic L24-22®, and anise oil.

Another embodiment comprises cyclosporin A, Surfonic L24-3®, and anise oil.

Another embodiment comprises cyclosporin A, Surfonic L24-4®, and anise oil.

Another embodiment comprises cyclosporin A, Surfonic L24-5®, and anise oil.

Another embodiment comprises cyclosporin A, Surfonic L24-7®, and anise oil.

Another embodiment comprises cyclosporin A, Surfonic L24-7.1®, and anise oil.

Another embodiment comprises cyclosporin A, Surfonic L24-9®, and anise oil.

Another embodiment comprises cyclosporin A, Surfonic LF-17®, and anise oil.

Another embodiment comprises cyclosporin A, Surfonic LF-18®, and anise oil.

Another embodiment comprises cyclosporin A, Surfonic LF-37®, and anise oil.

Another embodiment comprises cyclosporin A, Surfonic LF-41®, and anise oil.

Another embodiment comprises cyclosporin A, Surfonic LF-42®, and anise oil.

Another embodiment comprises cyclosporin A, Surfonic LF-47®, and anise oil.

Another embodiment comprises cyclosporin A, Surfonic LF-68®, and anise oil.

Another embodiment comprises cyclosporin A, Surfonic N-10®, and anise oil.

Another embodiment comprises cyclosporin A, Surfonic N-100®, and anise oil.

Another embodiment comprises cyclosporin A, Surfonic N-102®, and anise oil.

Another embodiment comprises cyclosporin A, Surfonic N-120®, and anise oil.

Another embodiment comprises cyclosporin A, Surfonic N-150®, and anise oil.

Another embodiment comprises cyclosporin A, Surfonic N-200®, and anise oil.

Another embodiment comprises cyclosporin A, Surfonic N-300®, and anise oil.

Another embodiment comprises cyclosporin A, Surfonic N-31.5®, and anise oil.

Another embodiment comprises cyclosporin A, Surfonic N-40®, and anise oil.

Another embodiment comprises cyclosporin A, Surfonic N-400®, and anise oil.

Another embodiment comprises cyclosporin A, Surfonic N-60®, and anise oil.

Another embodiment comprises cyclosporin A, Surfonic N-85®, and anise oil.

Another embodiment comprises cyclosporin A, Surfonic N-95®, and anise oil.

Another embodiment comprises cyclosporin A, Surfonic NB-18®, and anise oil.

Another embodiment comprises cyclosporin A, Surfonic NB-23®, and anise oil.

Another embodiment comprises cyclosporin A, Surfonic NB-25®, and anise oil.

Another embodiment comprises cyclosporin A, Surfonic NB-307®, and anise oil.

Another embodiment comprises cyclosporin A, Surfonic NB-31®, and anise oil.

Another embodiment comprises cyclosporin A, Surfonic NB-41®, and anise oil.

Another embodiment comprises cyclosporin A, Surfonic NB-43®, and anise oil.

Another embodiment comprises cyclosporin A, Surfonic OP-100®, and anise oil.

Another embodiment comprises cyclosporin A, Surfonic OP-120®, and anise oil.

Another embodiment comprises cyclosporin A, Surfonic OP-15®, and anise oil.

Another embodiment comprises cyclosporin A, Surfonic OP-35®, and anise oil.

Another embodiment comprises cyclosporin A, Surfonic OP-70®, and anise.oil.

Another embodiment comprises cyclosporin A, Surfonic P1®, and anise oil.

Another embodiment comprises cyclosporin A, Surfonic P3®, and anise oil.

Another embodiment comprises cyclosporin A, Surfonic P5®, and anise oil.

Another embodiment comprises cyclosporin A, Surfonic P6®, and anise oil.

Another embodiment comprises cyclosporin A, Surfonic POA-17R2®, and anise oil.

Another embodiment comprises cyclosporin A, Surfonic POA-25R2®, and anise oil.

Another embodiment comprises cyclosporin A, Surfonic TDA-11®, and anise oil.

Another embodiment comprises cyclosporin A, Surfonic TDA-3B®, and anise oil.

Another embodiment comprises cyclosporin A, Surfonic TDA-6®, and anise oil.

Another embodiment comprises cyclosporin A, Surfonic TDA-6/88®, and anise oil.

Another embodiment comprises cyclosporin A, Surfonic TDA-8®, and anise oil.

Another embodiment comprises cyclosporin A, Surfonic TDA-8/90®, and anise oil.

Another embodiment comprises cyclosporin A, Surfonic TDA-9®, and anise oil.

Another embodiment comprises cyclosporin A, Surfonic TX-CCN®, and anise oil.

Another embodiment comprises cyclosporin A, Tagat CH 40®, and anise oil.

Another embodiment comprises cyclosporin A, Tagat CH 60®, and anise oil.

Another embodiment comprises cyclosporin A, Tagat L 2®, and anise oil.

Another embodiment comprises cyclosporin A, Tagat S®, and anise oil.

Another embodiment comprises cyclosporin A, Tagat S 2®, and anise oil.

Another embodiment comprises cyclosporin A, Tally 100 Plus®, and anise oil.

Another embodiment comprises cyclosporin A, T-Det A106®, and anise oil.

Another embodiment comprises cyclosporin A, T-Det A109®, and anise oil.

Another embodiment comprises cyclosporin A, T-Det A136®, and anise oil.

Another embodiment comprises cyclosporin A, T-Det A139®, and anise oil.

Another embodiment comprises cyclosporin A, T-Det A2412®, and anise oil.

Another embodiment comprises cyclosporin A, T-Det A243®, and anise oil.

Another embodiment comprises cyclosporin A, T-Det A247®, and anise oil.

Another embodiment comprises cyclosporin A, T-Det A249®, and anise oil.

Another embodiment comprises cyclosporin A, T-Det A467®, and anise oil.

Another embodiment comprises cyclosporin A, T-Det COE®, and anise oil.

Another embodiment comprises cyclosporin A, T-Det DD-10®, and anise oil.

Another embodiment comprises cyclosporin A, T-Det DD-5®, and anise oil.

Another embodiment comprises cyclosporin A, T-Det DD-7®, and anise oil.

Another embodiment comprises cyclosporin A, T-Det EPO-104®, and anise oil.

Another embodiment comprises cyclosporin A, T-Det EPO-61®, and anise oil.

Another embodiment comprises cyclosporin A, T-Det EPO-62®, and anise oil.

Another embodiment comprises cyclosporin A, T-Det EPO-64®, and anise oil.

Another embodiment comprises cyclosporin A, T-Det N-1.5®, and anise oil.

Another embodiment comprises cyclosporin A, T-Det N-10.5®, and anise oil.

Another embodiment comprises cyclosporin A, T-Det N-100®, and anise oil.

Another embodiment comprises cyclosporin A, T-Det N-1007®, and anise oil.

Another embodiment comprises cyclosporin A, T-Det N-12®, and anise oil.

Another embodiment comprises cyclosporin A, T-Det N-14®, and anise oil.

Another embodiment comprises cyclosporin A, T-Det N-20®, and anise oil.

Another embodiment comprises cyclosporin A, T-Det N-30®, and anise oil.

Another embodiment comprises cyclosporin A, T-Det N-307®, and anise oil.

Another embodiment comprises cyclosporin A, T-Det N-4®, and anise oil.

Another embodiment comprises cyclosporin A, T-Det N-40®, and anise oil.

Another embodiment comprises cyclosporin A, T-Det N-407®, and anise oil.

Another embodiment comprises cyclosporin A, T-Det N-50®, and anise oil.

Another embodiment comprises cyclosporin A, T-Det N-507®, and anise oil.

Another embodiment comprises cyclosporin A, T-Det N-6®, and anise oil.

Another embodiment comprises cyclosporin A, T-Det N-70®, and anise oil.

Another embodiment comprises cyclosporin A, T-Det N-8®, and anise oil.

Another embodiment comprises cyclosporin A, T-Det N-9.5®, and anise oil.

Another embodiment comprises cyclosporin A, T-Det O-12®, and anise oil.

Another embodiment comprises cyclosporin A, T-Det O-165®, and anise oil.

Another embodiment comprises cyclosporin A, T-Det O-307®, and anise oil.

Another embodiment comprises cyclosporin A, T-Det O-40®, and anise oil.

Another embodiment comprises cyclosporin A, T-Det O-407®, and anise oil.

Another embodiment comprises cyclosporin A, T-Det O-6®, and anise oil.

Another embodiment comprises cyclosporin A, T-Det O-8®, and anise oil.

Another embodiment comprises cyclosporin A, T-Det O-9®, and anise oil.

Another embodiment comprises cyclosporin A, Tegin®, and anise oil.

Another embodiment comprises cyclosporin A, Tergitol 15-S-12®, and anise oil.

Another embodiment comprises cyclosporin A, Tergitol 15-S-15®, and anise oil.

Another embodiment comprises cyclosporin A, Tergitol 15-S-20®, and anise oil.

Another embodiment comprises cyclosporin A, Tergitol 15-S-20 (80AQ)®, and anise oil.

Another embodiment comprises cyclosporin A, Tergitol 15-S-3®, and anise oil.

Another embodiment comprises cyclosporin A, Tergitol 15-S-30®, and anise oil.

Another embodiment comprises cyclosporin A, Tergitol 15-S-40®, and anise oil.

Another embodiment comprises cyclosporin A, Tergitol 15-S-40 (70AQ)®, and anise oil.

Another embodiment comprises cyclosporin A, Tergitol 15-S-5®, and anise oil.

Another embodiment comprises cyclosporin A, Tergitol 15-S-7®, and anise oil.

Another embodiment comprises cyclosporin A, Tergitol 15-S-9®, and anise oil.

Another embodiment comprises cyclosporin A, Tergitol L-101®, and anise oil.

Another embodiment comprises cyclosporin A, Tergitol L-61®, and anise oil.

Another embodiment comprises cyclosporin A, Tergitol L-62®, and anise oil.

Another embodiment comprises cyclosporin A, Tergitol L-64®, and anise oil.

Another embodiment comprises cyclosporin A, Tergitol L-81®, and anise oil.

Another embodiment comprises cyclosporin A, Tergitol NP-10®, and anise oil.

Another embodiment comprises cyclosporin A, Tergitol NP-11®, and anise oil.

Another embodiment comprises cyclosporin A, Tergitol NP-12®, and anise oil.

Another embodiment comprises cyclosporin A, Tergitol NP-13®, and anise oil.

Another embodiment comprises cyclosporin A, Tergitol NP-15®, and anise oil.

Another embodiment comprises cyclosporin A, Tergitol NP-30®, and anise oil.

Another embodiment comprises cyclosporin A, Tergitol NP-30 (70 AQ)®, and anise oil.

Another embodiment comprises cyclosporin A, Tergitol NP-4®, and anise oil.

Another embodiment comprises cyclosporin A, Tergitol NP-40®, and anise oil.

Another embodiment comprises cyclosporin A, Tergitol NP-40 (70 AQ)®, and anise oil.

Another embodiment comprises cyclosporin A, Tergitol NP-50 (70 AQ)®, and anise oil.

Another embodiment comprises cyclosporin A, Tergitol NP-55 (70 AQ)®, and anise oil.

Another embodiment comprises cyclosporin A, Tergitol NP-6®, and anise oil.

Another embodiment comprises cyclosporin A, Tergitol NP-7®, and anise oil.

Another embodiment comprises cyclosporin A, Tergitol NP-70 (70 AQ)®, and anise oil.

Another embodiment comprises cyclosporin A, Tergitol NP-8®, and anise oil.

Another embodiment comprises cyclosporin A, Tergitol NP-9®, and anise oil.

Another embodiment comprises cyclosporin A, Tergitol NP-9.5®, and anise oil.

Another embodiment comprises cyclosporin A, Tergitol TMN-10®, and anise oil.

Another embodiment comprises cyclosporin A, Tergitol TMN-3®, and anise oil.

Another embodiment comprises cyclosporin A, Tergitol TMN-6®, and anise oil.

Another embodiment comprises cyclosporin A, Tergitol XD®, and anise oil.

Another embodiment comprises cyclosporin A, Tergitol XH®, and anise oil.

Another embodiment comprises cyclosporin A, Tergitol XJ®, and anise oil.

Another embodiment comprises cyclosporin A, Tetronic 1107®, and anise oil.

Another embodiment comprises cyclosporin A, Tetronic 1307®, and anise oil.

Another embodiment comprises cyclosporin A, Tetronic 150R1®, and anise oil.

Another embodiment comprises cyclosporin A, Tetronic 304®, and anise oil.

Another embodiment comprises cyclosporin A, Tetronic 701®, and anise oil.

Another embodiment comprises cyclosporin A, Tetronic 901®, and anise oil.

Another embodiment comprises cyclosporin A, Tetronic 904®, and anise oil.

Another embodiment comprises cyclosporin A, Tetronic 908®, and anise oil.

Another embodiment comprises cyclosporin A, Tetronic 90R4®, and anise oil.

Another embodiment comprises cyclosporin A, Tomadol 1-3®, and anise oil.

Another embodiment comprises cyclosporin A, Tomadol 1-5®, and anise oil.

Another embodiment comprises cyclosporin A, Tomadol 1-7®, and anise oil.

Another embodiment comprises cyclosporin A, Tomadol 23-1®, and anise oil.

Another embodiment comprises cyclosporin A, Tomadol 23-3®, and anise oil.

Another embodiment comprises cyclosporin A, Tomadol 23-5®, and anise oil.

Another embodiment comprises cyclosporin A, Tomadol 23-6.5®, and anise oil.

Another embodiment comprises cyclosporin A, Tomadol 25-12®, and anise oil.

Another embodiment comprises cyclosporin A, Tomadol 25-3®, and anise oil.

Another embodiment comprises cyclosporin A, Tomadol 25-7®, and anise oil.

Another embodiment comprises cyclosporin A, Tomadol 25-9®, and anise oil.

Another embodiment comprises cyclosporin A, Tomadol 45-13®, and anise oil.

Another embodiment comprises cyclosporin A, Tomadol 45-2.5®, and anise oil.

Another embodiment comprises cyclosporin A, Tomadol 45-7®, and anise oil.

Another embodiment comprises cyclosporin A, Tomadol 91-2.5®, and anise oil.

Another embodiment comprises cyclosporin A, Tomadol 91-6®, and anise oil.

Another embodiment comprises cyclosporin A, Tomadol 91-8®, and anise oil.

Another embodiment comprises cyclosporin A, Triton X-100®, and anise oil.

Another embodiment comprises cyclosporin A, Triton X-102®, and anise oil.

Another embodiment comprises cyclosporin A, Triton X-114®, and anise oil.

Another embodiment comprises cyclosporin A, Triton X-15®, and anise oil.

Another embodiment comprises cyclosporin A, Triton X-165 (70%)®, and anise oil.

Another embodiment comprises cyclosporin A, Triton X-207®, and anise oil.

Another embodiment comprises cyclosporin A, Triton X-305 (70%)®, and anise oil.

Another embodiment comprises cyclosporin A, Triton X-35®, and anise oil.

Another embodiment comprises cyclosporin A, Triton X-405 (70%)®, and anise oil.

Another embodiment comprises cyclosporin A, Triton X-45®, and anise oil.

Another embodiment comprises cyclosporin A, Triton X-705 (70%)®, and anise oil.

Another embodiment comprises cyclosporin A, Volpo S-10®, and anise oil.

Another embodiment comprises cyclosporin A, Volpo S-2®, and anise oil.

Another embodiment comprises cyclosporin A, Volpo S-20®, and anise oil.

Another embodiment comprises cyclosporin A, Volpo-10®, and anise oil.

Another embodiment comprises cyclosporin A, Volpo-20®, and anise oil.

Another embodiment comprises cyclosporin A, Volpo-3®, and anise oil.

Another embodiment comprises cyclosporin A, Volpo-5®, and anise oil.

Another embodiment comprises cyclosporin A, Abex 2515®, and Castor oil.

Another embodiment comprises cyclosporin A, Abex 2525/40®, and Castor oil.

Another embodiment comprises cyclosporin A, Abex 2535®, and Castor oil.

Another embodiment comprises cyclosporin A, Abex 2545®, and Castor oil.

Another embodiment comprises cyclosporin A, Acconon E®, and Castor oil.

Another embodiment comprises cyclosporin A, Alfonic 1216CO-9®, and Castor oil.

Another embodiment comprises cyclosporin A, Alfonic 1412-3®, and Castor oil.

Another embodiment comprises cyclosporin A, Alfonic TDA-12®, and Castor oil.

Another embodiment comprises cyclosporin A, Alfonic TDA-3®, and Castor oil.

Another embodiment comprises cyclosporin A, Alfonic TDA-4®, and Castor oil.

Another embodiment comprises cyclosporin A, Alfonic TDA-6®, and Castor oil.

Another embodiment comprises cyclosporin A, Alfonic TDA-7®, and Castor oil.

Another embodiment comprises cyclosporin A, Alfonic TDA-8®, and Castor oil.

Another embodiment comprises cyclosporin A, Alfonic TDA-9®, and Castor oil.

Another embodiment comprises cyclosporin A, Alkamuls 400-DO®, and Castor oil.

Another embodiment comprises cyclosporin A, Alkamuls 400-MO/E®, and Castor oil.

Another embodiment comprises cyclosporin A, Ameroxol OE-10®, and Castor oil.

Another embodiment comprises cyclosporin A, Ameroxol OE-2®, and Castor oil.

Another embodiment comprises cyclosporin A, Ameroxol OE-20®, and Castor oil.

Another embodiment comprises cyclosporin A, Armix 176®, and Castor oil.

Another embodiment comprises cyclosporin A, Armix 180-C®, and Castor oil.

Another embodiment comprises cyclosporin A, Armix 183®, and Castor oil.

Another embodiment comprises cyclosporin A, Brij 35®, and Castor oil.

Another embodiment comprises cyclosporin A, Brij 35 Liquid®, and Castor oil.

Another embodiment comprises cyclosporin A, Brij 35 SP®, and Castor oil.

Another embodiment comprises cyclosporin A, Canasol MJ 2109®, and Castor oil.

Another embodiment comprises cyclosporin A, Canasol MJ 52®, and Castor oil.

Another embodiment comprises cyclosporin A, Canasol R 3600®, and Castor oil.

Another embodiment comprises cyclosporin A, Canasol R 3603®, and Castor oil.

Another embodiment comprises cyclosporin A, Canasol R 4000 H®, and Castor oil.

Another embodiment comprises cyclosporin A, Cedepal CA-210®, and Castor oil.

Another embodiment comprises cyclosporin A, Cedepal CA-520®, and Castor oil.

Another embodiment comprises cyclosporin A, Cedepal CA-630®, and Castor oil.

Another embodiment comprises cyclosporin A, Cedepal CA-720®, and Castor oil.

Another embodiment comprises cyclosporin A, Cedepal CA-890®, and Castor oil.

Another embodiment comprises cyclosporin A, Cedepal CA-897®, and Castor oil.

Another embodiment comprises cyclosporin A, Cedepal CO-210®, and Castor oil.

Another embodiment comprises cyclosporin A, Cedepal CO-430®, and Castor oil.

Another embodiment comprises cyclosporin A, Cedepal CO-530®, and Castor oil.

Another embodiment comprises cyclosporin A, Cedepal CO-630®, and Castor oil.

Another embodiment comprises cyclosporin A, Cedepal CO-710®, and Castor oil.

Another embodiment comprises cyclosporin A, Cedepal CO-730®, and Castor oil.

Another embodiment comprises cyclosporin A, Cedepal CO-880®, and Castor oil.

Another embodiment comprises cyclosporin A, Cedepal CO-887®, and Castor oil.

Another embodiment comprises cyclosporin A, Cedepal CO-890®, and Castor oil.

Another embodiment comprises cyclosporin A, Cedepal CO-897®, and Castor oil.

Another embodiment comprises cyclosporin A, Cedepal CO-970®, and Castor oil.

Another embodiment comprises cyclosporin A, Cedepal CO-977®, and Castor oil.

Another embodiment comprises cyclosporin A, Cedepal CO-990®, and Castor oil.

Another embodiment comprises cyclosporin A, Cedepal CO-997®, and Castor oil.

Another embodiment comprises cyclosporin A, Chemal BP 261®, and Castor oil.

Another embodiment comprises cyclosporin A, Chemal BP 262®, and Castor oil.

Another embodiment comprises cyclosporin A, Chemal BP 3172®, and Castor oil.

Another embodiment comprises cyclosporin A, Chemal BP 3174®, and Castor oil.

Another embodiment comprises cyclosporin A, Chemal DA-4®, and Castor oil.

Another embodiment comprises cyclosporin A, Chemal DA-6®, and Castor oil.

Another embodiment comprises cyclosporin A, Chemal DA-9®, and Castor oil.

Another embodiment comprises cyclosporin A, Chemal LA-23®, and Castor oil.

Another embodiment comprises cyclosporin A, Chemal LA-4®, and Castor oil.

Another embodiment comprises cyclosporin A, Chemal LA-9®, and Castor oil.

Another embodiment comprises cyclosporin A, Chemal OA-20G®, and Castor oil.

Another embodiment comprises cyclosporin A, Chemal OA-5®, and Castor oil.

Another embodiment comprises cyclosporin A, Chemal OA-9®, and Castor oil.

Another embodiment comprises cyclosporin A, Chemal TDA-12®, and Castor oil.

Another embodiment comprises cyclosporin A, Chemal TDA-15®, and Castor oil.

Another embodiment comprises cyclosporin A, Chemal TDA-3®, and Castor oil.

Another embodiment comprises cyclosporin A, Chemal TDA-6®, and Castor oil.

Another embodiment comprises cyclosporin A, Chemal TDA-9®, and Castor oil.

Another embodiment comprises cyclosporin A, Chemax CO-16®, and Castor oil.

Another embodiment comprises cyclosporin A, Chemax CO-200/50®, and Castor oil.

Another embodiment comprises cyclosporin A, Chemax CO-25®, and Castor oil.

Another embodiment comprises cyclosporin A, Chemax CO-30®, and Castor oil.

Another embodiment comprises cyclosporin A, Chemax CO-36®, and Castor oil.

Another embodiment comprises cyclosporin A, Chemax CO-40®, and Castor oil.

Another embodiment comprises cyclosporin A, Chemax CO-5®, and Castor oil.

Another embodiment comprises cyclosporin A, Chemax CO-80®, and Castor oil.

Another embodiment comprises cyclosporin A, Chemax DNP-150/50®, and Castor oil.

Another embodiment comprises cyclosporin A, Chemax DNP-18®, and Castor oil.

Another embodiment comprises cyclosporin A, Chemax DNP-8®, and Castor oil.

Another embodiment comprises cyclosporin A, Chemax E 200 ML®, and Castor oil.

Another embodiment comprises cyclosporin A, Chemax E 200 MO®, and Castor oil.

Another embodiment comprises cyclosporin A, Chemax E 400 ML®, and Castor oil.

Another embodiment comprises cyclosporin A, Chemax E 400 MO®, and Castor oil.

Another embodiment comprises cyclosporin A, Chemax E 600 ML®, and Castor oil.

Another embodiment comprises cyclosporin A, Chemax E 600 MO®, and Castor oil.

Another embodiment comprises cyclosporin A, Chemax E1000 MO®, and Castor oil.

Another embodiment comprises cyclosporin A, Chemax E1000 MS®, and Castor oil.

Another embodiment comprises cyclosporin A, Chemax E200 MS®, and Castor oil.

Another embodiment comprises cyclosporin A, Chemax E400 MS®, and Castor oil.

Another embodiment comprises cyclosporin A, Chemax E600 MS®, and Castor oil.

Another embodiment comprises cyclosporin A, Chemax HCO-16®, and Castor oil.

Another embodiment comprises cyclosporin A, Chemax HCO-200/50®, and Castor oil.

Another embodiment comprises cyclosporin A, Chemax HCO-25®, and Castor oil.

Another embodiment comprises cyclosporin A, Chemax HCO-5®, and Castor oil.

Another embodiment comprises cyclosporin A, Chemax TO-16®, and Castor oil.

Another embodiment comprises cyclosporin A, Chemax TO-8®, and Castor oil.

Another embodiment comprises cyclosporin A, Colalube 3409®, and Castor oil.

Another embodiment comprises cyclosporin A, Colalube 3411®, and Castor oil.

Another embodiment comprises cyclosporin A, Colalube 3413®, and Castor oil.

Another embodiment comprises cyclosporin A, Colalube 3414®, and Castor oil.

Another embodiment comprises cyclosporin A, Colalube 3415®, and Castor oil.

Another embodiment comprises cyclosporin A, Colalube 3416®, and Castor oil.

Another embodiment comprises cyclosporin A, Colalube 3417®, and Castor oil.

Another embodiment comprises cyclosporin A, Delonic NPE-100®, and Castor oil.

Another embodiment comprises cyclosporin A, Delonic NPE-30®, and Castor oil.

Another embodiment comprises cyclosporin A, Delonic NPE-40®, and Castor oil.

Another embodiment comprises cyclosporin A, Delonic OPE-10®, and Castor oil.

Another embodiment comprises cyclosporin A, Delonic OPE-12®, and Castor oil.

Another embodiment comprises cyclosporin A, Delonic OPE-30®, and Castor oil.

Another embodiment comprises cyclosporin A, Delonic OPE-40®, and Castor oil.

Another embodiment comprises cyclosporin A, Delonic OPE-5®, and Castor oil.

Another embodiment comprises cyclosporin A, Delonic OPE-7.5®, and Castor oil.

Another embodiment comprises cyclosporin A, DePeg 16-CO®, and Castor oil.

Another embodiment comprises cyclosporin A, DePeg 25-CO®, and Castor oil.

Another embodiment comprises cyclosporin A, DePeg 30-CO®, and Castor oil.

Another embodiment comprises cyclosporin A, DePeg 40-CO®, and Castor oil.

Another embodiment comprises cyclosporin A, DePeg 5-CO®, and Castor oil.

Another embodiment comprises cyclosporin A, DePeg 80-CO®, and Castor oil.

Another embodiment comprises cyclosporin A, DeSonic S-100®, and Castor oil.

Another embodiment comprises cyclosporin A, DeSonic S-114®, and Castor oil.

Another embodiment comprises cyclosporin A, DeSonic S-405®, and Castor oil.

Another embodiment comprises cyclosporin A, DeSonic S-45®, and Castor oil.

Another embodiment comprises cyclosporin A, DeSonic TDA-9®, and Castor oil.

Another embodiment comprises cyclosporin A, DeThox Acid O-14®, and Castor oil.

Another embodiment comprises cyclosporin A, DeThox Acid O-16®, and Castor oil.

Another embodiment comprises cyclosporin A, DeThox Acid O-9®, and Castor oil.

Another embodiment comprises cyclosporin A, DeThox Acid TO-14®, and Castor oil.

Another embodiment comprises cyclosporin A, DeThox Acid TO-16.5®, and Castor oil.

Another embodiment comprises cyclosporin A, DeThox Acid TO-8.5®, and Castor oil.

Another embodiment comprises cyclosporin A, Dur-Em 114®, and Castor oil.

Another embodiment comprises cyclosporin A, Dur-Em 117®, and Castor oil.

Another embodiment comprises cyclosporin A, Dur-Em 204®, and Castor oil.

Another embodiment comprises cyclosporin A, Dur-Em 207®, and Castor oil.

Another embodiment comprises cyclosporin A, Dur-Em 207E®, and Castor oil.

Another embodiment comprises cyclosporin A, Emerest 2704®, and Castor oil.

Another embodiment comprises cyclosporin A, Emerest 2712®, and Castor oil.

Another embodiment comprises cyclosporin A, Emerest 2715®, and Castor oil.

Another embodiment comprises cyclosporin A, Ethal EH-2®, and Castor oil.

Another embodiment comprises cyclosporin A, Ethal EH-5®, and Castor oil.

Another embodiment comprises cyclosporin A, Ethal OA-23®, and Castor oil.

Another embodiment comprises cyclosporin A, Ethosperse CA-20®, and Castor oil.

Another embodiment comprises cyclosporin A, Ethosperse LA-12®, and Castor oil.

Another embodiment comprises cyclosporin A, Ethosperse LA-23®, and Castor oil.

Another embodiment comprises cyclosporin A, Ethosperse LA-4®, and Castor oil.

Another embodiment comprises cyclosporin A, Ethox 3095®, and Castor oil.

Another embodiment comprises cyclosporin A, Ethox CO-16®, and Castor oil.

Another embodiment comprises cyclosporin A, Ethox CO-200®, and Castor oil.

Another embodiment comprises cyclosporin A, Ethox CO-25®, and Castor oil.

Another embodiment comprises cyclosporin A, Ethox CO-30®, and Castor oil.

Another embodiment comprises cyclosporin A, Ethox CO-36®, and Castor oil.

Another embodiment comprises cyclosporin A, Ethox CO-40®, and Castor oil.

Another embodiment comprises cyclosporin A, Ethox CO-5®, and Castor oil.

Another embodiment comprises cyclosporin A, Ethox DO-14®, and Castor oil.

Another embodiment comprises cyclosporin A, Ethox DO-9®, and Castor oil.

Another embodiment comprises cyclosporin A, Ethox MA-8®, and Castor oil.

Another embodiment comprises cyclosporin A, Ethox MI-14®, and Castor oil.

Another embodiment comprises cyclosporin A, Ethox ML-14®, and Castor oil.

Another embodiment comprises cyclosporin A, Ethox ML-5®, and Castor oil.

Another embodiment comprises cyclosporin A, Ethox MO-14®, and Castor oil.

Another embodiment comprises cyclosporin A, Ethox MO-9®, and Castor oil.

Another embodiment comprises cyclosporin A, Ethox MS-14®, and Castor oil.

Another embodiment comprises cyclosporin A, Ethox MS-23®, and Castor oil.

Another embodiment comprises cyclosporin A, Ethox MS-40®, and Castor oil.

Another embodiment comprises cyclosporin A, Ethox MS-8®, and Castor oil.

Another embodiment comprises cyclosporin A, Ethox TO-16®, and Castor oil.

Another embodiment comprises cyclosporin A, Ethox TO-8®, and Castor oil.

Another embodiment comprises cyclosporin A, Ethylan SN-120®, and Castor oil.

Another embodiment comprises cyclosporin A, Ethylan SN-70®, and Castor oil.

Another embodiment comprises cyclosporin A, Ethylan SN-90®, and Castor oil.

Another embodiment comprises cyclosporin A, Flexricin 13®, and Castor oil.

Another embodiment comprises cyclosporin A, Flexricin 15®, and Castor oil.

Another embodiment comprises cyclosporin A, Genapol 1261®, and Castor oil.

Another embodiment comprises cyclosporin A, Generol 122 E25®, and Castor oil.

Another embodiment comprises cyclosporin A, Glicepol 160®, and Castor oil.

Another embodiment comprises cyclosporin A, Glicepol 560®, and Castor oil.

Another embodiment comprises cyclosporin A, Glicepol GMS 20®, and Castor oil.

Another embodiment comprises cyclosporin A, Hetoxide BN-13®, and Castor oil.

Another embodiment comprises cyclosporin A, Hetoxide DNP-4®, and Castor oil.

Another embodiment comprises cyclosporin A, Hetoxide DNP-9.6®, and Castor oil.

Another embodiment comprises cyclosporin A, Hetoxide G-26®, and Castor oil.

Another embodiment comprises cyclosporin A, Hetoxide G-7®, and Castor oil.

Another embodiment comprises cyclosporin A, Hetoxide GT-20®, and Castor oil.

Another embodiment comprises cyclosporin A, Hetoxide HC-40®, and Castor oil.

Another embodiment comprises cyclosporin A, Hetoxol C-24®, and Castor oil.

Another embodiment comprises cyclosporin A, Hetoxol CA-10®, and Castor oil.

Another embodiment comprises cyclosporin A, Hetoxol CA-2®, and Castor oil.

Another embodiment comprises cyclosporin A, Hetoxol CA-20®, and Castor oil.

Another embodiment comprises cyclosporin A, Hetoxol CAWS®, and Castor oil.

Another embodiment comprises cyclosporin A, Hetoxol CD-3®, and Castor oil.

Another embodiment comprises cyclosporin A, Hetoxol CD-4®, and Castor oil.

Another embodiment comprises cyclosporin A, Hetoxol CS-15®, and Castor oil.

Another embodiment comprises cyclosporin A, Hetoxol CS-20®, and Castor oil.

Another embodiment comprises cyclosporin A, Hetoxol CS-20D®, and Castor oil.

Another embodiment comprises cyclosporin A, Hetoxol CS-25®, and Castor oil.

Another embodiment comprises cyclosporin A, Hetoxol CS-50®, and Castor oil.

Another embodiment comprises cyclosporin A, Hetoxol CS-9®, and Castor oil.

Another embodiment comprises cyclosporin A, Hetoxol CSA-15®, and Castor oil.

Another embodiment comprises cyclosporin A, Hetoxol D®, and Castor oil.

Another embodiment comprises cyclosporin A, Hetoxol G®, and Castor oil.

Another embodiment comprises cyclosporin A, Hetoxol J®, and Castor oil.

Another embodiment comprises cyclosporin A, Hetoxol L®, and Castor oil.

Another embodiment comprises cyclosporin A, Hetoxol L-23®, and Castor oil.

Another embodiment comprises cyclosporin A, Hetoxol L-4®, and Castor oil.

Another embodiment comprises cyclosporin A, Hetoxol L-9®, and Castor oil.

Another embodiment comprises cyclosporin A, Hetoxol LS-9®, and Castor oil.

Another embodiment comprises cyclosporin A, Hetoxol M-3®, and Castor oil.

Another embodiment comprises cyclosporin A, Hetoxol OA-10 Special®, and Castor oil.

Another embodiment comprises cyclosporin A, Hetoxol OA-20 Special®, and Castor oil.

Another embodiment comprises cyclosporin A, Hetoxol OA-3 Special®, and Castor oil.

Another embodiment comprises cyclosporin A, Hetoxol OA-5 Special®, and Castor oil.

Another embodiment comprises cyclosporin A, Hetoxol OL-10®, and Castor oil.

Another embodiment comprises cyclosporin A, Hetoxol OL-2®, and Castor oil.

Another embodiment comprises cyclosporin A, Hetoxol OL-23®, and Castor oil.

Another embodiment comprises cyclosporin A, Hetoxol OL-4®, and Castor oil.

Another embodiment comprises cyclosporin A, Hetoxol OL-40®, and Castor oil.

Another embodiment comprises cyclosporin A, Hetoxol OL-5®, and Castor oil.

Another embodiment comprises cyclosporin A, Hetoxol PLA®, and Castor oil.

Another embodiment comprises cyclosporin A, Hetoxol SP-15®, and Castor oil.

Another embodiment comprises cyclosporin A, Hetoxol STA-10®, and Castor oil.

Another embodiment comprises cyclosporin A, Hetoxol STA-2®, and Castor oil.

Another embodiment comprises cyclosporin A, Hetoxol STA-30®, and Castor oil.

Another embodiment comprises cyclosporin A, Hetoxol TD-12®, and Castor oil.

Another embodiment comprises cyclosporin A, Hetoxol TD-18®, and Castor oil.

Another embodiment comprises cyclosporin A, Hetoxol TD-3®, and Castor oil.

Another embodiment comprises cyclosporin A, Hetoxol TD-6®, and Castor oil.

Another embodiment comprises cyclosporin A, Hetoxol TD-9®, and Castor oil.

Another embodiment comprises cyclosporin A, Hetoxol TDEP-15®, and Castor oil.

Another embodiment comprises cyclosporin A, Hetoxol TDEP-63®, and Castor oil.

Another embodiment comprises cyclosporin A, Iconol 24-12®, and Castor oil.

Another embodiment comprises cyclosporin A, Iconol 24-23®, and Castor oil.

Another embodiment comprises cyclosporin A, Iconol 24-4®, and Castor oil.

Another embodiment comprises cyclosporin A, Iconol DA-4®, and Castor oil.

Another embodiment comprises cyclosporin A, Iconol DA-6®, and Castor oil.

Another embodiment comprises cyclosporin A, Iconol TDA-10®, and Castor oil.

Another embodiment comprises cyclosporin A, Iconol TDA-3®, and Castor oil.

Another embodiment comprises cyclosporin A, Iconol TDA-6®, and Castor oil.

Another embodiment comprises cyclosporin A, Iconol TDA-8-90%®, and Castor oil.

Another embodiment comprises cyclosporin A, Iconol TDA-9®, and Castor oil.

Another embodiment comprises cyclosporin A, Igepal CA-210®, and Castor oil.

Another embodiment comprises cyclosporin A, Igepal CA-407®, and Castor oil.

Another embodiment comprises cyclosporin A, Igepal CA-420®, and Castor oil.

Another embodiment comprises cyclosporin A, Igepal CA-520®, and Castor oil.

Another embodiment comprises cyclosporin A, Igepal CA-620®, and Castor oil.

Another embodiment comprises cyclosporin A, Igepal CA-630®, and Castor oil.

Another embodiment comprises cyclosporin A, Igepal CA-720®, and Castor oil.

Another embodiment comprises cyclosporin A, Igepal CA-877®, and Castor oil.

Another embodiment comprises cyclosporin A, Igepal CA-887®, and Castor oil.

Another embodiment comprises cyclosporin A, Igepal CA-897®, and Castor oil.

Another embodiment comprises cyclosporin A, Igepal CO-210®, and Castor oil.

Another embodiment comprises cyclosporin A, Igepal CO-430®, and Castor oil.

Another embodiment comprises cyclosporin A, Igepal CO-520®, and Castor oil.

Another embodiment comprises cyclosporin A, Igepal CO-530®, and Castor oil.

Another embodiment comprises cyclosporin A, Igepal CO-610®, and Castor oil.

Another embodiment comprises cyclosporin A, Igepal CO-630®, and Castor oil.

Another embodiment comprises cyclosporin A, Igepal CO-630 Special®, and Castor oil.

Another embodiment comprises cyclosporin A, Igepal CO-660®, and Castor oil.

Another embodiment comprises cyclosporin A, Igepal CO-710®, and Castor oil.

Another embodiment comprises cyclosporin A, Igepal CO-720®, and Castor oil.

Another embodiment comprises cyclosporin A, Igepal CO-730®, and Castor oil.

Another embodiment comprises cyclosporin A, Igepal CO-738®, and Castor oil.

Another embodiment comprises cyclosporin A, Igepal CO-850®, and Castor oil.

Another embodiment comprises cyclosporin A, Igepal CO-880®, and Castor oil.

Another embodiment comprises cyclosporin A, Igepal CO-887®, and Castor oil.

Another embodiment comprises cyclosporin A, Igepal CO-890®, and Castor oil.

Another embodiment comprises cyclosporin A, Igepal CO-897®, and Castor oil.

Another embodiment comprises cyclosporin A, Igepal CO-970®, and Castor oil.

Another embodiment comprises cyclosporin A, Igepal CO-977®, and Castor oil.

Another embodiment comprises cyclosporin A, Igepal CO-987®, and Castor oil.

Another embodiment comprises cyclosporin A, Igepal CO-990 FLK®, and Castor oil.

Another embodiment comprises cyclosporin A, Igepal CO-997®, and Castor oil.

Another embodiment comprises cyclosporin A, Igepal DM-430®, and Castor oil.

Another embodiment comprises cyclosporin A, Igepal DM-530®, and Castor oil.

Another embodiment comprises cyclosporin A, Igepal DM-710®, and Castor oil.

Another embodiment comprises cyclosporin A, Igepal DM-730®, and Castor oil.

Another embodiment comprises cyclosporin A, Igepal DM-880®, and Castor oil.

Another embodiment comprises cyclosporin A, Igepal DM-970 FLK®, and Castor oil.

Another embodiment comprises cyclosporin A, Igepal OD-410®, and Castor oil.

Another embodiment comprises cyclosporin A, Igepal RC-520®, and Castor oil.

Another embodiment comprises cyclosporin A, Igepal RC-620®, and Castor oil.

Another embodiment comprises cyclosporin A, Igepal RC-630®, and Castor oil.

Another embodiment comprises cyclosporin A, Lexemul CS-20®, and Castor oil.

Another embodiment comprises cyclosporin A, Lipo GMS 450®, and Castor oil.

Another embodiment comprises cyclosporin A, Lipo Polyglycol 1000®, and Castor oil.

Another embodiment comprises cyclosporin A, Lipo Polyglycol 200®, and Castor oil.

Another embodiment comprises cyclosporin A, Lipo Polyglycol 300®, and Castor oil.

Another embodiment comprises cyclosporin A, Lipo Polyglycol 3350®, and Castor oil.

Another embodiment comprises cyclosporin A, Lipo Polyglycol 400®, and Castor oil.

Another embodiment comprises cyclosporin A, Lipo Polyglycol 600®, and Castor oil.

Another embodiment comprises cyclosporin A, Lipocol C-10®, and Castor oil.

Another embodiment comprises cyclosporin A, Lipocol C-2®, and Castor oil.

Another embodiment comprises cyclosporin A, Lipocol C-20®, and Castor oil.

Another embodiment comprises cyclosporin A, Lipocol L-12®, and Castor oil.

Another embodiment comprises cyclosporin A, Lipocol L-23®, and Castor oil.

Another embodiment comprises cyclosporin A, Lipocol L-4®, and Castor oil.

Another embodiment comprises cyclosporin A, Lipocol O-10®, and Castor oil.

Another embodiment comprises cyclosporin A, Lipocol O-2®, and Castor oil.

Another embodiment comprises cyclosporin A, Lipocol O-20®, and Castor oil.

Another embodiment comprises cyclosporin A, Lipocol O-5®, and Castor oil.

Another embodiment comprises cyclosporin A, Lipocol S-10®, and Castor oil.

Another embodiment comprises cyclosporin A, Lipocol S-2®, and Castor oil.

Another embodiment comprises cyclosporin A, Lipocol S-20®, and Castor oil.

Another embodiment comprises cyclosporin A, Lipocol SC-15®, and Castor oil.

Another embodiment comprises cyclosporin A, Lipocol SC-20®, and Castor oil.

Another embodiment comprises cyclosporin A, Lipocol SC-4®, and Castor oil.

Another embodiment comprises cyclosporin A, Lipocol TD-12®, and Castor oil.

Another embodiment comprises cyclosporin A, Lumulse 40-O K®, and Castor oil.

Another embodiment comprises cyclosporin A, Lumulse 42-O K®, and Castor oil.

Another embodiment comprises cyclosporin A, Lumulse GMT-40®, and Castor oil.

Another embodiment comprises cyclosporin A, Lumulse PEG 1450®, and Castor oil.

Another embodiment comprises cyclosporin A, Lumulse PEG 1450 NF®, and Castor oil.

Another embodiment comprises cyclosporin A, Lumulse PEG 8000®, and Castor oil.

Another embodiment comprises cyclosporin A, Lumulse PGO®, and Castor oil.

Another embodiment comprises cyclosporin A, Lumulse POE (2) Oleyl Amine®, and Castor oil.

Another embodiment comprises cyclosporin A, Magrabar PGO-104®, and Castor oil.

Another embodiment comprises cyclosporin A, Mapeg 200 ML®, and Castor oil.

Another embodiment comprises cyclosporin A, Mapeg 400 MO®, and Castor oil.

Another embodiment comprises cyclosporin A, Mapeg 400 MOT®, and Castor oil.

Another embodiment comprises cyclosporin A, Mapeg 400DO®, and Castor oil.

Another embodiment comprises cyclosporin A, Mapeg 400DOT®, and Castor oil.

Another embodiment comprises cyclosporin A, Mapeg 600DOT®, and Castor oil.

Another embodiment comprises cyclosporin A, Marlox B 24/80®, and Castor oil.

Another embodiment comprises cyclosporin A, Marlox FK 86®, and Castor oil.

Another embodiment comprises cyclosporin A, Marlox MO 154®, and Castor oil.

Another embodiment comprises cyclosporin A, Mazox CDA CG®, and Castor oil.

Another embodiment comprises cyclosporin A, Monoglycerides Glyceryl Monestearate®, and Castor oil.

Another embodiment comprises cyclosporin A, Myrj 45®, and Castor oil.

Another embodiment comprises cyclosporin A, Myrj 52®, and Castor oil.

Another embodiment comprises cyclosporin A, Myrj 52S®, and Castor oil.

Another embodiment comprises cyclosporin A, Myrj 53®, and Castor oil.

Another embodiment comprises cyclosporin A, Myrj 59®, and Castor oil.

Another embodiment comprises cyclosporin A, Norfox NP-12®, and Castor oil.

Another embodiment comprises cyclosporin A, Norfox NP-4®, and Castor oil.

Another embodiment comprises cyclosporin A, Norfox NP-6®, and Castor oil.

Another embodiment comprises cyclosporin A, Norfox NP-887®, and Castor oil.

Another embodiment comprises cyclosporin A, Norfox NP-9®, and Castor oil.

Another embodiment comprises cyclosporin A, Norfox NP-977®, and Castor oil.

Another embodiment comprises cyclosporin A, Norfox OP-100®, and Castor oil.

Another embodiment comprises cyclosporin A, Norfox OP-102®, and Castor oil.

Another embodiment comprises cyclosporin A, Norfox OP-114®, and Castor oil.

Another embodiment comprises cyclosporin A, Norfox OP-45®, and Castor oil.

Another embodiment comprises cyclosporin A, Pegosperse 100-L®, and Castor oil.

Another embodiment comprises cyclosporin A, Pegosperse 100-O®, and Castor oil.

Another embodiment comprises cyclosporin A, Pegosperse 100-S®, and Castor oil.

Another embodiment comprises cyclosporin A, Pegosperse 1500-MS®, and Castor oil.

Another embodiment comprises cyclosporin A, Pegosperse 1750-MS®, and Castor oil.

Another embodiment comprises cyclosporin A, Pegosperse 200 DL®, and Castor oil.

Another embodiment comprises cyclosporin A, Pegosperse 200-ML®, and Castor oil.

Another embodiment comprises cyclosporin A, Pegosperse 400-DL®, and Castor oil.

Another embodiment comprises cyclosporin A, Pegosperse 400-DO®, and Castor oil.

Another embodiment comprises cyclosporin A, Pegosperse 400-DS®, and Castor oil.

Another embodiment comprises cyclosporin A, Pegosperse 400-ML®, and Castor oil.

Another embodiment comprises cyclosporin A, Pegosperse 400-MO®, and Castor oil.

Another embodiment comprises cyclosporin A, Pegosperse 400-MOT®, and Castor oil.

Another embodiment comprises cyclosporin A, Pegosperse 400-MS®, and Castor oil.

Another embodiment comprises cyclosporin A, Pegosperse 50-DS®, and Castor oil.

Another embodiment comprises cyclosporin A, Pegosperse 50-MS®, and Castor oil.

Another embodiment comprises cyclosporin A, Pegosperse 600-ML®, and Castor oil.

Another embodiment comprises cyclosporin A, Pegosperse 600-MS®, and Castor oil.

Another embodiment comprises cyclosporin A, Plurafac A-38®, and Castor oil.

Another embodiment comprises cyclosporin A, Plurafac B-25-5®, and Castor oil.

Another embodiment comprises cyclosporin A, Plurafac B-26®, and Castor oil.

Another embodiment comprises cyclosporin A, Plurafac D-25®, and Castor oil.

Another embodiment comprises cyclosporin A, Plurafac LF 131®, and Castor oil.

Another embodiment comprises cyclosporin A, Plurafac LF 4030®, and Castor oil.

Another embodiment comprises cyclosporin A, Plurafac LF 7000®, and Castor oil.

Another embodiment comprises cyclosporin A, Plurafac RA-20®, and Castor oil.

Another embodiment comprises cyclosporin A, Plurafac RA-30®, and Castor oil.

Another embodiment comprises cyclosporin A, Plurafac RA-40®, and Castor oil.

Another embodiment comprises cyclosporin A, Pluronic 10R5®, and Castor oil.

Another embodiment comprises cyclosporin A, Pluronic 17R2®, and Castor oil.

Another embodiment comprises cyclosporin A, Pluronic 17R4®, and Castor oil.

Another embodiment comprises cyclosporin A, Pluronic 25R2®, and Castor oil.

Another embodiment comprises cyclosporin A, Pluronic 25R4®, and Castor oil.

Another embodiment comprises cyclosporin A, Pluronic 31R1®, and Castor oil.

Another embodiment comprises cyclosporin A, Pluronic F108®, and Castor oil.

Another embodiment comprises cyclosporin A, Pluronic F127®, and Castor oil.

Another embodiment comprises cyclosporin A, Pluronic F38®, and Castor oil.

Another embodiment comprises cyclosporin A, Pluronic F68®, and Castor oil.

Another embodiment comprises cyclosporin A, Pluronic F68LF®, and Castor oil.

Another embodiment comprises cyclosporin A, Pluronic F77®, and Castor oil.

Another embodiment comprises cyclosporin A, Pluronic F87®, and Castor oil.

Another embodiment comprises cyclosporin A, Pluronic F88®, and Castor oil.

Another embodiment comprises cyclosporin A, Pluronic F98®, and Castor oil.

Another embodiment comprises cyclosporin A, Pluronic L10®, and Castor oil.

Another embodiment comprises cyclosporin A, Pluronic L101®, and Castor oil.

Another embodiment comprises cyclosporin A, Pluronic L121®, and Castor oil.

Another embodiment comprises cyclosporin A, Pluronic L31®, and Castor oil.

Another embodiment comprises cyclosporin A, Pluronic L35®, and Castor oil.

Another embodiment comprises cyclosporin A, Pluronic L43®, and Castor oil.

Another embodiment comprises cyclosporin A, Pluronic L44®, and Castor oil.

Another embodiment comprises cyclosporin A, Pluronic L61®, and Castor oil.

Another embodiment comprises cyclosporin A, Pluronic L62®, and Castor oil.

Another embodiment comprises cyclosporin A, Pluronic L62D®, and Castor oil.

Another embodiment comprises cyclosporin A, Pluronic L62LF®, and Castor oil.

Another embodiment comprises cyclosporin A, Pluronic L64®, and Castor oil.

Another embodiment comprises cyclosporin A, Pluronic L81®, and Castor oil.

Another embodiment comprises cyclosporin A, Pluronic L92®, and Castor oil.

Another embodiment comprises cyclosporin A, Pluronic P103®, and Castor oil.

Another embodiment comprises cyclosporin A, Pluronic P104®, and Castor oil.

Another embodiment comprises cyclosporin A, Pluronic P105®, and Castor oil.

Another embodiment comprises cyclosporin A, Pluronic P123®, and Castor oil.

Another embodiment comprises cyclosporin A, Pluronic P65®, and Castor oil.

Another embodiment comprises cyclosporin A, Pluronic P84®, and Castor oil.

Another embodiment comprises cyclosporin A, Pluronic P85®, and Castor oil.

Another embodiment comprises cyclosporin A, Polyaldo 10-2-P®, and Castor oil.

Another embodiment comprises cyclosporin A, Polyaldo DGDO®, and Castor oil.

Another embodiment comprises cyclosporin A, Polyaldo HGDS®, and Castor oil.

Another embodiment comprises cyclosporin A, Polyaldo TGMS®, and Castor oil.

Another embodiment comprises cyclosporin A, Protegin V®, and Castor oil.

Another embodiment comprises cyclosporin A, Protegin W®, and Castor oil.

Another embodiment comprises cyclosporin A, Protegin WX®, and Castor oil.

Another embodiment comprises cyclosporin A, Protegin XV®, and Castor oil.

Another embodiment comprises cyclosporin A, Rhodasurf BC-420®, and Castor oil.

Another embodiment comprises cyclosporin A, Rhodasurf BC-610®, and Castor oil.

Another embodiment comprises cyclosporin A, Rhodasurf BC-630®, and Castor oil.

Another embodiment comprises cyclosporin A, Rhodasurf BC-720®, and Castor oil.

Another embodiment comprises cyclosporin A, Rhodasurf BC-840®, and Castor oil.

Another embodiment comprises cyclosporin A, Rhodasurf L 1®, and Castor oil.

Another embodiment comprises cyclosporin A, Rhodasurf L 12®, and Castor oil.

Another embodiment comprises cyclosporin A, Rhodasurf L 2®, and Castor oil.

Another embodiment comprises cyclosporin A, Rhodasurf L 20®, and Castor oil.

Another embodiment comprises cyclosporin A, Rhodasurf L 3®, and Castor oil.

Another embodiment comprises cyclosporin A, Rhodasurf L 30®, and Castor oil.

Another embodiment comprises cyclosporin A, Rhodasurf L 4®, and Castor oil.

Another embodiment comprises cyclosporin A, Rhodasurf L 50®, and Castor oil.

Another embodiment comprises cyclosporin A, Rhodasurf L 7/90®, and Castor oil.

Another embodiment comprises cyclosporin A, Rhodasurf LA-3®, and Castor oil.

Another embodiment comprises cyclosporin A, Rhodasurf LA-7®, and Castor oil.

Another embodiment comprises cyclosporin A, Rhodasurf LAN-23®, and Castor oil.

Another embodiment comprises cyclosporin A, Rhodasurf ON-870®, and Castor oil.

Another embodiment comprises cyclosporin A, Rhodasurf ON-877®, and Castor oil.

Another embodiment comprises cyclosporin A, Rhodasurf TDA-8/5®, and Castor oil.

Another embodiment comprises cyclosporin A, Ritapro 100®, and Castor oil.

Another embodiment comprises cyclosporin A, Ritapro 165®, and Castor oil.

Another embodiment comprises cyclosporin A, Ritapro 200®, and Castor oil.

Another embodiment comprises cyclosporin A, Ritapro 300®, and Castor oil.

Another embodiment comprises cyclosporin A, Ritoleth 10®, and Castor oil.

Another embodiment comprises cyclosporin A, Ritoleth 2®, and Castor oil.

Another embodiment comprises cyclosporin A, Ritoleth 20®, and Castor oil.

Another embodiment comprises cyclosporin A, Ritoleth 5®, and Castor oil.

Another embodiment comprises cyclosporin A, Santone 10-10-0®, and Castor oil.

Another embodiment comprises cyclosporin A, Santone 3-1-S XTR®, and Castor oil.

Another embodiment comprises cyclosporin A, Santone 3-1-SH®, and Castor oil.

Another embodiment comprises cyclosporin A, Santone 8-1-O®, and Castor oil.

Another embodiment comprises cyclosporin A, Solulan 16®, and Castor oil.

Another embodiment comprises cyclosporin A, Solulan 75®, and Castor oil.

Another embodiment comprises cyclosporin A, Solulan C-24®, and Castor oil.

Another embodiment comprises cyclosporin A, Solulan L-575®, and Castor oil.

Another embodiment comprises cyclosporin A, Standamox CAW®, and Castor oil.

Another embodiment comprises cyclosporin A, Surfine AZI-A®, and Castor oil.

Another embodiment comprises cyclosporin A, Surfonic CO-15®, and Castor oil.

Another embodiment comprises cyclosporin A, Surfonic CO-25®, and Castor oil.

Another embodiment comprises cyclosporin A, Surfonic CO-30®, and Castor oil.

Another embodiment comprises cyclosporin A, Surfonic CO-36®, and Castor oil.

Another embodiment comprises cyclosporin A, Surfonic CO-42®, and Castor oil.

Another embodiment comprises cyclosporin A, Surfonic E400-MO®, and Castor oil.

Another embodiment comprises cyclosporin A, Surfonic L12-3®, and Castor oil.

Another embodiment comprises cyclosporin A, Surfonic L12-6®, and Castor oil.

Another embodiment comprises cyclosporin A, Surfonic L12-8®, and Castor oil.

Another embodiment comprises cyclosporin A, Surfonic L24-12®, and Castor oil.

Another embodiment comprises cyclosporin A, Surfonic L24-2®, and Castor oil.

Another embodiment comprises cyclosporin A, Surfonic L24-22®, and Castor oil.

Another embodiment comprises cyclosporin A, Surfonic L24-3®, and Castor oil.

Another embodiment comprises cyclosporin A, Surfonic L24-4®, and Castor oil.

Another embodiment comprises cyclosporin A, Surfonic L24-5®, and Castor oil.

Another embodiment comprises cyclosporin A, Surfonic L24-7®, and Castor oil.

Another embodiment comprises cyclosporin A, Surfonic L24-7.1®, and Castor oil.

Another embodiment comprises cyclosporin A, Surfonic L24-9®, and Castor oil.

Another embodiment comprises cyclosporin A, Surfonic LF-17®, and Castor oil.

Another embodiment comprises cyclosporin A, Surfonic LF-18®, and Castor oil.

Another embodiment comprises cyclosporin A, Surfonic LF-37®, and Castor oil.

Another embodiment comprises cyclosporin A, Surfonic LF-41®, and Castor oil.

Another embodiment comprises cyclosporin A, Surfonic LF-42®, and Castor oil.

Another embodiment comprises cyclosporin A, Surfonic LF-47®, and Castor oil.

Another embodiment comprises cyclosporin A, Surfonic LF-68®, and Castor oil.

Another embodiment comprises cyclosporin A, Surfonic N-10®, and Castor oil.

Another embodiment comprises cyclosporin A, Surfonic N-100®, and Castor oil.

Another embodiment comprises cyclosporin A, Surfonic N-102®, and Castor oil.

Another embodiment comprises cyclosporin A, Surfonic N-120®, and Castor oil.

Another embodiment comprises cyclosporin A, Surfonic N-150®, and Castor oil.

Another embodiment comprises cyclosporin A, Surfonic N-200®, and Castor oil.

Another embodiment comprises cyclosporin A, Surfonic N-300®, and Castor oil.

Another embodiment comprises cyclosporin A, Surfonic N-31.5®, and Castor oil.

Another embodiment comprises cyclosporin A, Surfonic N-40®, and Castor oil.

Another embodiment comprises cyclosporin A, Surfonic N-400®, and Castor oil.

Another embodiment comprises cyclosporin A, Surfonic N-60®, and Castor oil.

Another embodiment comprises cyclosporin A, Surfonic N-85®, and Castor oil.

Another embodiment comprises cyclosporin A, Surfonic N-95®, and Castor oil.

Another embodiment comprises cyclosporin A, Surfonic NB-18®, and Castor oil.

Another embodiment comprises cyclosporin A, Surfonic NB-23®, and Castor oil.

Another embodiment comprises cyclosporin A, Surfonic NB-25®, and Castor oil.

Another embodiment comprises cyclosporin A, Surfonic NB-307®, and Castor oil.

Another embodiment comprises cyclosporin A, Surfonic NB-31®, and Castor oil.

Another embodiment comprises cyclosporin A, Surfonic NB-41®, and Castor oil.

Another embodiment comprises cyclosporin A, Surfonic NB-43®, and Castor oil.

Another embodiment comprises cyclosporin A, Surfonic OP-100®, and Castor oil.

Another embodiment comprises cyclosporin A, Surfonic OP-120®, and Castor oil.

Another embodiment comprises cyclosporin A, Surfonic OP-15®, and Castor oil.

Another embodiment comprises cyclosporin A, Surfonic OP-35®, and Castor oil.

Another embodiment comprises cyclosporin A, Surfonic OP-70®, and Castor oil.

Another embodiment comprises cyclosporin A, Surfonic P1®, and Castor oil.

Another embodiment comprises cyclosporin A, Sunfonic P3®, and Castor oil.

Another embodiment comprises cyclosporin A, Sunfonic P5®, and Castor oil.

Another embodiment comprises cyclosporin A, Surfonic P6®, and Castor oil.

Another embodiment comprises cyclosporin A, Surfonic POA-17R2®, and Castor oil.

Another embodiment comprises cyclosporin A, Surfonic POA-25R2®, and Castor oil.

Another embodiment comprises cyclosporin A, Surfonic TDA-11®, and Castor oil.

Another embodiment comprises cyclosporin A, Surfonic TDA-3B®, and Castor oil.

Another embodiment comprises cyclosporin A, Surfonic TDA-6®, and Castor oil.

Another embodiment comprises cyclosporin A, Surfonic TDA-6/88®, and Castor oil.

Another embodiment comprises cyclosporin A, Surfonic TDA-8®, and Castor oil.

Another embodiment comprises cyclosporin A, Surfonic TDA-8/90®, and Castor oil.

Another embodiment comprises cyclosporin A, Surfonic TDA-9®, and Castor oil.

Another embodiment comprises cyclosporin A, Surfonic TX-CCN®, and Castor oil.

Another embodiment comprises cyclosporin A, Tagat CH 40®, and Castor oil.

Another embodiment comprises cyclosporin A, Tagat CH 60®, and Castor oil.

Another embodiment comprises cyclosporin A, Tagat L 2®, and Castor oil.

Another embodiment comprises cyclosporin A, Tagat S®, and Castor oil.

Another embodiment comprises cyclosporin A, Tagat S 2®, and Castor oil.

Another embodiment comprises cyclosporin A, Tally 100 Plus®, and Castor oil.

Another embodiment comprises cyclosporin A, T-Det A106®, and Castor oil.

Another embodiment comprises cyclosporin A, T-Det A109®, and Castor oil.

Another embodiment comprises cyclosporin A, T-Det A136®, and Castor oil.

Another embodiment comprises cyclosporin A, T-Det A139®, and Castor oil.

Another embodiment comprises cyclosporin A, T-Det A2412®, and Castor oil.

Another embodiment comprises cyclosporin A, T-Det A243®, and Castor oil.

Another embodiment comprises cyclosporin A, T-Det A247®, and Castor oil.

Another embodiment comprises cyclosporin A, T-Det A249®, and Castor oil.

Another embodiment comprises cyclosporin A, T-Det A467®, and Castor oil.

Another embodiment comprises cyclosporin A, T-Det COE®, and Castor oil.

Another embodiment comprises cyclosporin A, T-Det DD-10®, and Castor oil.

Another embodiment comprises cyclosporin A, T-Det DD-5®, and Castor oil.

Another embodiment comprises cyclosporin A, T-Det DD-7®, and Castor oil.

Another embodiment comprises cyclosporin A, T-Det EPO-104®, and Castor oil.

Another embodiment comprises cyclosporin A, T-Det EPO-61®, and Castor oil.

Another embodiment comprises cyclosporin A, T-Det EPO-62®, and Castor oil.

Another embodiment comprises cyclosporin A, T-Det EPO-64®, and Castor oil.

Another embodiment comprises cyclosporin A, T-Det N-1.5®, and Castor oil.

Another embodiment comprises cyclosporin A, T-Det N-10.5®, and Castor oil.

Another embodiment comprises cyclosporin A, T-Det N-100®, and Castor oil.

Another embodiment comprises cyclosporin A, T-Det N-1007®, and Castor oil.

Another embodiment comprises cyclosporin A, T-Det N-12®, and Castor oil.

Another embodiment comprises cyclosporin A, T-Det N-14®, and Castor oil.

Another embodiment comprises cyclosporin A, T-Det N-20®, and Castor oil.

Another embodiment comprises cyclosporin A, T-Det N-30®, and Castor oil.

Another embodiment comprises cyclosporin A, T-Det N-307®, and Castor oil.

Another embodiment comprises cyclosporin A, T-Det N-4®, and Castor oil.

Another embodiment comprises cyclosporin A, T-Det N-40®, and Castor oil.

Another embodiment comprises cyclosporin A, T-Det N-407®, and Castor oil.

Another embodiment comprises cyclosporin A, T-Det N-50®, and Castor oil.

Another embodiment comprises cyclosporin A, T-Det N-507®, and Castor oil.

Another embodiment comprises cyclosporin A, T-Det N-6®, and Castor oil.

Another embodiment comprises cyclosporin A, T-Det N-70®, and Castor oil.

Another embodiment comprises cyclosporin A, T-Det N-8®, and Castor oil.

Another embodiment comprises cyclosporin A, T-Det N-9.5®, and Castor oil.

Another embodiment comprises cyclosporin A, T-Det O-12®, and Castor oil.

Another embodiment comprises cyclosporin A, T-Det O-165®, and Castor oil.

Another embodiment comprises cyclosporin A, T-Det O-307®, and Castor oil.

Another embodiment comprises cyclosporin A, T-Det O-40®, and Castor oil.

Another embodiment comprises cyclosporin A, T-Det O-407®, and Castor oil.

Another embodiment comprises cyclosporin A, T-Det O-6®, and Castor oil.

Another embodiment comprises cyclosporin A, T-Det O-8®, and Castor oil.

Another embodiment comprises cyclosporin A, T-Det O-9®, and Castor oil.

Another embodiment comprises cyclosporin A, Tegin®, and Castor oil.

Another embodiment comprises cyclosporin A, Tergitol 15-S-12®, and Castor oil.

Another embodiment comprises cyclosporin A, Tergitol 15-S-15®, and Castor oil.

Another embodiment comprises cyclosporin A, Tergitol 15-S-20®, and Castor oil.

Another embodiment comprises cyclosporin A, Tergitol 15-S-20 (80AQ)®, and Castor oil.

Another embodiment comprises cyclosporin A, Tergitol 15-S-3®, and Castor oil.

Another embodiment comprises cyclosporin A, Tergitol 15-S-30®, and Castor oil.

Another embodiment comprises cyclosporin A, Tergitol 15-S-40®, and Castor oil.

Another embodiment comprises cyclosporin A, Tergitol 15-S-40 (70AQ)®, and Castor oil.

Another embodiment comprises cyclosporin A, Tergitol 15-S-5®, and Castor oil.

Another embodiment comprises cyclosporin A, Tergitol 15-S-7®, and Castor oil.

Another embodiment comprises cyclosporin A, Tergitol 15-S-9®, and Castor oil.

Another embodiment comprises cyclosporin A, Tergitol L-101®, and Castor oil.

Another embodiment comprises cyclosporin A, Tergitol L-61®, and Castor oil.

Another embodiment comprises cyclosporin A, Tergitol L-62®, and Castor oil.

Another embodiment comprises cyclosporin A, Tergitol L-64®, and Castor oil.

Another embodiment comprises cyclosporin A, Tergitol L-81®, and Castor oil.

Another embodiment comprises cyclosporin A, Tergitol NP-10®, and Castor oil.

Another embodiment comprises cyclosporin A, Tergitol NP-11®, and Castor oil.

Another embodiment comprises cyclosporin A, Tergitol NP-12®, and Castor oil.

Another embodiment comprises cyclosporin A, Tergitol NP-13®, and Castor oil.

Another embodiment comprises cyclosporin A, Tergitol NP-15®, and Castor oil.

Another embodiment comprises cyclosporin A, Tergitol NP-30®, and Castor oil.

Another embodiment comprises cyclosporin A, Tergitol NP-30 (70 AQ)®, and Castor oil.

Another embodiment comprises cyclosporin A, Tergitol NP-4®, and Castor oil.

Another embodiment comprises cyclosporin A, Tergitol NP-40®, and Castor oil.

Another embodiment comprises cyclosporin A, Tergitol NP-40 (70 AQ)®, and Castor oil.

Another embodiment comprises cyclosporin A, Tergitol NP-50 (70 AQ)®, and Castor oil.

Another embodiment comprises cyclosporin A, Tergitol NP-55 (70 AQ)®, and Castor oil.

Another embodiment comprises cyclosporin A, Tergitol NP-6®, and Castor oil.

Another embodiment comprises cyclosporin A, Tergitol NP-7®, and Castor oil.

Another embodiment comprises cyclosporin A, Tergitol NP-70 (70 AQ)®, and Castor oil.

Another embodiment comprises cyclosporin A, Tergitol NP-8®, and Castor oil.

Another embodiment comprises cyclosporin A, Tergitol NP-9®, and Castor oil.

Another embodiment comprises cyclosporin A, Tergitol NP-9.5®, and Castor oil.

Another embodiment comprises cyclosporin A, Tergitol TMN-10®, and Castor oil.

Another embodiment comprises cyclosporin A, Tergitol TMN-3®, and Castor oil.

Another embodiment comprises cyclosporin A, Tergitol TMN-6®, and Castor oil.

Another embodiment comprises cyclosporin A, Tergitol XD®, and Castor oil.

Another embodiment comprises cyclosporin A, Tergitol XH®, and Castor oil.

Another embodiment comprises cyclosporin A, Tergitol XJ®, and Castor oil.

Another embodiment comprises cyclosporin A, Tetronic 1107®, and Castor oil.

Another embodiment comprises cyclosporin A, Tetronic 1307®, and Castor oil.

Another embodiment comprises cyclosporin A, Tetronic 150R1®, and Castor oil.

Another embodiment comprises cyclosporin A, Tetronic 304®, and Castor oil.

Another embodiment comprises cyclosporin A, Tetronic 701®, and Castor oil.

Another embodiment comprises cyclosporin A, Tetronic 901®, and Castor oil.

Another embodiment comprises cyclosporin A, Tetronic 904®, and Castor oil.

Another embodiment comprises cyclosporin A, Tetronic 908®, and Castor oil.

Another embodiment comprises cyclosporin A, Tetronic 90R4®, and Castor oil.

Another embodiment comprises cyclosporin A, Tomadol 1-3®, and Castor oil.

Another embodiment comprises cyclosporin A, Tomadol 1-5®, and Castor oil.

Another embodiment comprises cyclosporin A, Tomadol 1-7®, and Castor oil.

Another embodiment comprises cyclosporin A, Tomadol 23-1®, and Castor oil.

Another embodiment comprises cyclosporin A, Tomadol 23-3®, and Castor oil.

Another embodiment comprises cyclosporin A, Tomadol 23-5®, and Castor oil.

Another embodiment comprises cyclosporin A, Tomadol 23-6.5®, and Castor oil.

Another embodiment comprises cyclosporin A, Tomadol 25-12®, and Castor oil.

Another embodiment comprises cyclosporin A, Tomadol 25-3®, and Castor oil.

Another embodiment comprises cyclosporin A, Tomadol 25-7®, and Castor oil.

Another embodiment comprises cyclosporin A, Tomadol 25-9®, and Castor oil.

Another embodiment comprises cyclosporin A, Tomadol 45-13®, and Castor oil.

Another embodiment comprises cyclosporin A, Tomadol 45-2.5®, and Castor oil.

Another embodiment comprises cyclosporin A, Tomadol 45-7®, and Castor oil.

Another embodiment comprises cyclosporin A, Tomadol 91-2.5®, and Castor oil.

Another embodiment comprises cyclosporin A, Tomadol 91-6®, and Castor oil.

Another embodiment comprises cyclosporin A, Tomadol 91-8®, and Castor oil.

Another embodiment comprises cyclosporin A, Triton X-100®, and Castor oil.

Another embodiment comprises cyclosporin A, Triton X-102®, and Castor oil.

Another embodiment comprises cyclosporin A, Triton X-114®, and Castor oil.

Another embodiment comprises cyclosporin A, Triton X-15®, and Castor oil.

Another embodiment comprises cyclosporin A, Triton X-165 (70%)®, and Castor oil.

Another embodiment comprises cyclosporin A, Triton X-207®, and Castor oil.

Another embodiment comprises cyclosporin A, Triton X-305 (70%)®, and Castor oil.

Another embodiment comprises cyclosporin A, Triton X-35®, and Castor oil.

Another embodiment comprises cyclosporin A, Triton X-405 (70%)®, and Castor oil.

Another embodiment comprises cyclosporin A, Triton X-45®, and Castor oil.

Another embodiment comprises cyclosporin A, Triton X-705 (70%)®, and Castor oil.

Another embodiment comprises cyclosporin A, Volpo S-10®, and Castor oil.

Another embodiment comprises cyclosporin A, Volpo S-2®, and Castor oil.

Another embodiment comprises cyclosporin A, Volpo S-20®, and Castor oil.

Another embodiment comprises cyclosporin A, Volpo-10®, and Castor oil.

Another embodiment comprises cyclosporin A, Volpo-20®, and Castor oil.

Another embodiment comprises cyclosporin A, Volpo-3®, and Castor oil.

Another embodiment comprises cyclosporin A, Volpo-5®, and Castor oil.

Another embodiment comprises cyclosporin A, Abex 2515/®, and clove oil.

Another embodiment comprises cyclosporin A, Abex 2525/40®, and clove oil.

Another embodiment comprises cyclosporin A, Abex 2535®, and clove oil.

Another embodiment comprises cyclosporin A, Abex 2545®, and clove oil.

Another embodiment comprises cyclosporin A, Acconon E®, and clove oil.

Another embodiment comprises cyclosporin A, Alfonic 1216CO-9®, and clove oil.

Another embodiment comprises cyclosporin A, Alfonic 1412-3®, and clove oil.

Another embodiment comprises cyclosporin A, Alfonic TDA-12®, and clove oil.

Another embodiment comprises cyclosporin A, Alfonic TDA-3®, and clove oil.

Another embodiment comprises cyclosporin A, Alfonic TDA-4®, and clove oil.

Another embodiment comprises cyclosporin A, Alfonic TDA-6®, and clove oil.

Another embodiment comprises cyclosporin A, Alfonic TDA-7®, and clove oil.

Another embodiment comprises cyclosporin A, Alfonic TDA-8®, and clove oil.

Another embodiment comprises cyclosporin A, Alfonic TDA-9®, and clove oil.

Another embodiment comprises cyclosporin A, Alkamuls 400-DO®, and clove oil.

Another embodiment comprises cyclosporin A, Alkamuls 400-MO/E®, and clove oil.

Another embodiment comprises cyclosporin A, Ameroxol OE-10®, and clove oil.

Another embodiment comprises cyclosporin A, Ameroxol OE-2®, and clove oil.

Another embodiment comprises cyclosporin A, Ameroxol OE-20®, and clove oil.

Another embodiment comprises cyclosporin A, Armix 176®, and clove oil.

Another embodiment comprises cyclosporin A, Armix 180-C®, and clove oil.

Another embodiment comprises cyclosporin A, Armix 183®, and clove oil.

Another embodiment comprises cyclosporin A, Brij 35®, and clove oil.

Another embodiment comprises cyclosporin A, Brij 35 Liquid®, and clove oil.

Another embodiment comprises cyclosporin A, Brij 35 SP®, and clove oil.

Another embodiment comprises cyclosporin A, Canasol MJ 2109®, and clove oil.

Another embodiment comprises cyclosporin A, Canasol MJ 52®, and clove oil.

Another embodiment comprises cyclosporin A, Canasol R 3600®, and clove oil.

Another embodiment comprises cyclosporin A, Canasol R 3603®, and clove oil.

Another embodiment comprises cyclosporin A, Canasol R 4000 H®, and clove oil.

Another embodiment comprises cyclosporin A, Cedepal CA-210®, and clove oil.

Another embodiment comprises cyclosporin A, Cedepal CA-520®, and clove oil.

Another embodiment comprises cyclosporin A, Cedepal CA-630®, and clove oil.

Another embodiment comprises cyclosporin A, Cedepal CA-720®, and clove oil.

Another embodiment comprises cyclosporin A, Cedepal CA-890®, and clove oil.

Another embodiment comprises cyclosporin A, Cedepal CA-897®, and clove oil.

Another embodiment comprises cyclosporin A, Cedepal CO-210®, and clove oil.

Another embodiment comprises cyclosporin A, Cedepal CO-430®, and clove oil.

Another embodiment comprises cyclosporin A, Cedepal CO-530®, and clove oil.

Another embodiment comprises cyclosporin A, Cedepal CO-630®, and clove oil.

Another embodiment comprises cyclosporin A, Cedepal CO-710®, and clove oil.

Another embodiment comprises cyclosporin A, Cedepal CO-730®, and clove oil.

Another embodiment comprises cyclosporin A, Cedepal CO-880®, and clove oil.

Another embodiment comprises cyclosporin A, Cedepal CO-887®, and clove oil.

Another embodiment comprises cyclosporin A, Cedepal CO-890®, and clove oil.

Another embodiment comprises cyclosporin A, Cedepal CO-897®, and clove oil.

Another embodiment comprises cyclosporin A, Cedepal CO-970®, and clove oil.

Another embodiment comprises cyclosporin A, Cedepal CO-977®, and clove oil.

Another embodiment comprises cyclosporin A, Cedepal CO-990®, and clove oil.

Another embodiment comprises cyclosporin A, Cedepal CO-997®, and clove oil.

Another embodiment comprises cyclosporin A, Chemal BP 261®, and clove oil.

Another embodiment comprises cyclosporin A, Chemal BP 262®, and clove oil.

Another embodiment comprises cyclosporin A, Chemal BP 3172®, and clove oil.

Another embodiment comprises cyclosporin A, Chemal BP 3174®, and clove oil.

Another embodiment comprises cyclosporin A, Chemal DA-4®, and clove oil.

Another embodiment comprises cyclosporin A, Chemal DA-6®, and clove oil.

Another embodiment comprises cyclosporin A, Chemal DA-9®, and clove oil.

Another embodiment comprises cyclosporin A, Chemal LA-23®, and clove oil.

Another embodiment comprises cyclosporin A, Chemal LA-4®, and clove oil.

Another embodiment comprises cyclosporin A, Chemal LA-9®, and clove oil.

Another embodiment comprises cyclosporin A, Chemal OA-20G®, and clove oil.

Another embodiment comprises cyclosporin A, Chemal OA-5®, and clove oil.

Another embodiment comprises cyclosporin A, Chemal OA-9®, and clove oil.

Another embodiment comprises cyclosporin A, Chemal TDA-12®, and clove oil.

Another embodiment comprises cyclosporin A, Chemal TDA-15®, and clove oil.

Another embodiment comprises cyclosporin A, Chemal TDA-3®, and clove oil.

Another embodiment comprises cyclosporin A, Chemal TDA-6®, and clove oil.

Another embodiment comprises cyclosporin A, Chemal TDA-9®, and clove oil.

Another embodiment comprises cyclosporin A, Chemax CO-16®, and clove oil.

Another embodiment comprises cyclosporin A, Chemax CO-200/50®, and clove oil.

Another embodiment comprises cyclosporin A, Chemax CO-25®, and clove oil.

Another embodiment comprises cyclosporin A, Chemax CO-30®, and clove oil.

Another embodiment comprises cyclosporin A, Chemax CO-36®, and clove oil.

Another embodiment comprises cyclosporin A, Chemax CO-40®, and clove oil.

Another embodiment comprises cyclosporin A, Chemax CO-5®, and clove oil.

Another embodiment comprises cyclosporin A, Chemax CO-80®, and clove oil.

Another embodiment comprises cyclosporin A, Chemax DNP-150/50®, and clove oil.

Another embodiment comprises cyclosporin A, Chemax DNP-18®, and clove oil.

Another embodiment comprises cyclosporin A, Chemax DNP-8®, and clove oil.

Another embodiment comprises cyclosporin A, Chemax E 200 ML®, and clove oil.

Another embodiment comprises cyclosporin A, Chemax E 200 MO®, and clove oil.

Another embodiment comprises cyclosporin A, Chemax E 400 ML®, and clove oil.

Another embodiment comprises cyclosporin A, Chemax E 400 MO®, and clove oil.

Another embodiment comprises cyclosporin A, Chemax E 600 ML®, and clove oil.

Another embodiment comprises cyclosporin A, Chemax E 600 MO®, and clove oil.

Another embodiment comprises cyclosporin A, Chemax E1000 MO®, and clove oil.

Another embodiment comprises cyclosporin A, Chemax E1000 MS®, and clove oil.

Another embodiment comprises cyclosporin A, Chemax E200 MS®, and clove oil.

Another embodiment comprises cyclosporin A, Chemax E400 MS®, and clove oil.

Another embodiment comprises cyclosporin A, Chemax E600 MS®, and clove oil.

Another embodiment comprises cyclosporin A, Chemax HCO-16®, and clove oil.

Another embodiment comprises cyclosporin A, Chemax HCO-200/50®, and clove oil.

Another embodiment comprises cyclosporin A, Chemax HCO-25®, and clove oil.

Another embodiment comprises cyclosporin A, Chemax HCO-5®, and clove oil.

Another embodiment comprises cyclosporin A, Chemax TO-16®, and clove oil.

Another embodiment comprises cyclosporin A, Chemax TO-8®, and clove oil.

Another embodiment comprises cyclosporin A, Colalube 3409®, and clove oil.

Another embodiment comprises cyclosporin A, Colalube 3411®, and clove oil.

Another embodiment comprises cyclosporin A, Colalube 3413®, and clove oil.

Another embodiment comprises cyclosporin A, Colalube 3414®, and clove oil.

Another embodiment comprises cyclosporin A, Colalube 3415®, and clove oil.

Another embodiment comprises cyclosporin A, Colalube 3416®, and clove oil.

Another embodiment comprises cyclosporin A, Colalube 3417®, and clove oil.

Another embodiment comprises cyclosporin A, Delonic NPE-100®, and clove oil.

Another embodiment comprises cyclosporin A, Delonic NPE-30®, and clove oil.

Another embodiment comprises cyclosporin A, Delonic NPE-40®, and clove oil.

Another embodiment comprises cyclosporin A, Delonic OPE-10®, and clove oil.

Another embodiment comprises cyclosporin A, Delonic OPE-12®, and clove oil.

Another embodiment comprises cyclosporin A, Delonic OPE-30®, and clove oil.

Another embodiment comprises cyclosporin A, Delonic OPE-40®, and clove oil.

Another embodiment comprises cyclosporin A, Delonic OPE-5®, and clove oil.

Another embodiment comprises cyclosporin A, Delonic OPE-7.5®, and clove oil.

Another embodiment comprises cyclosporin A, DePeg 16-CO®, and clove oil.

Another embodiment comprises cyclosporin A, DePeg 25-CO®, and clove oil.

Another embodiment comprises cyclosporin A, DePeg 30-CO®, and clove oil.

Another embodiment comprises cyclosporin A, DePeg 40-CO®, and clove oil.

Another embodiment comprises cyclosporin A, DePeg 5-CO®, and clove oil.

Another embodiment comprises cyclosporin A, DePeg 80-CO®, and clove oil.

Another embodiment comprises cyclosporin A, DeSonic S-100®, and clove oil.

Another embodiment comprises cyclosporin A, DeSonic S-114®, and clove oil.

Another embodiment comprises cyclosporin A, DeSonic S-405®, and clove oil.

Another embodiment comprises cyclosporin A, DeSonic S-45®, and clove oil.

Another embodiment comprises cyclosporin A, DeSonic TDA-9®, and clove oil.

Another embodiment comprises cyclosporin A, DeThox Acid O-14®, and clove oil.

Another embodiment comprises cyclosporin A, DeThox Acid O-16®, and clove oil.

Another embodiment comprises cyclosporin A, DeThox Acid O-9®, and clove oil.

Another embodiment comprises cyclosporin A, DeThox Acid TO-14®, and clove oil.

Another embodiment comprises cyclosporin A, DeThox Acid TO-16.5®, and clove oil.

Another embodiment comprises cyclosporin A, DeThox Acid TO-8.5®, and clove oil.

Another embodiment comprises cyclosporin A, Dur-Em 114®, and clove oil.

Another embodiment comprises cyclosporin A, Dur-Em 117®, and clove oil.

Another embodiment comprises cyclosporin A, Dur-Em 204®, and clove oil.

Another embodiment comprises cyclosporin A, Dur-Em 207®, and clove oil.

Another embodiment comprises cyclosporin A, Dur-Em 207E®, and clove oil.

Another embodiment comprises cyclosporin A, Emerest 2704®, and clove oil.

Another embodiment comprises cyclosporin A, Emerest 2712®, and clove oil.

Another embodiment comprises cyclosporin A, Emerest 2715®, and clove oil.

Another embodiment comprises cyclosporin A, Ethal EH-2®, and clove oil.

Another embodiment comprises cyclosporin A, Ethal EH-5®, and clove oil.

Another embodiment comprises cyclosporin A, Ethal OA-23®, and clove oil.

Another embodiment comprises cyclosporin A, Ethosperse CA-20®, and clove oil.

Another embodiment comprises cyclosporin A, Ethosperse LA-12®, and clove oil.

Another embodiment comprises cyclosporin A, Ethosperse LA-23®, and clove oil.

Another embodiment comprises cyclosporin A, Ethosperse LA-4®, and clove oil.

Another embodiment comprises cyclosporin A, Ethox 3095®, and clove oil.

Another embodiment comprises cyclosporin A, Ethox CO-16®, and clove oil.

Another embodiment comprises cyclosporin A, Ethox CO-200®, and clove oil.

Another embodiment comprises cyclosporin A, Ethox CO-25®, and clove oil.

Another embodiment comprises cyclosporin A, Ethox CO-30®, and clove oil.

Another embodiment comprises cyclosporin A, Ethox CO-36®, and clove oil.

Another embodiment comprises cyclosporin A, Ethox CO-40®, and clove oil.

Another embodiment comprises cyclosporin A, Ethox CO-5®, and clove oil.

Another embodiment comprises cyclosporin A, Ethox DO-14®, and clove oil.

Another embodiment comprises cyclosporin A, Ethox DO-9®, and clove oil.

Another embodiment comprises cyclosporin A, Ethox MA-8®, and clove oil.

Another embodiment comprises cyclosporin A, Ethox Ml-14®, and clove oil.

Another embodiment comprises cyclosporin A, Ethox ML-14®, and clove oil.

Another embodiment comprises cyclosporin A, Ethox ML-5®, and clove oil.

Another embodiment comprises cyclosporin A, Ethox MO-14®, and clove oil.

Another embodiment comprises cyclosporin A, Ethox MO-9®, and clove oil.

Another embodiment comprises cyclosporin A, Ethox MS-14®, and clove oil.

Another embodiment comprises cyclosporin A, Ethox MS-23®, and clove oil.

Another embodiment comprises cyclosporin A, Ethox MS-40®, and clove oil.

Another embodiment comprises cyclosporin A, Ethox MS-8®, and clove oil.

Another embodiment comprises cyclosporin A, Ethox TO-16®, and clove oil.

Another embodiment comprises cyclosporin A, Ethox TO-8®, and clove oil.

Another embodiment comprises cyclosporin A, Ethylan SN-120®, and clove oil.

Another embodiment comprises cyclosporin A, Ethylan SN-70®, and clove oil.

Another embodiment comprises cyclosporin A, Ethylan SN-90®, and clove oil.

Another embodiment comprises cyclosporin A, Flexricin 13®, and clove oil.

Another embodiment comprises cyclosporin A, Flexricin 15®, and clove oil.

Another embodiment comprises cyclosporin A, Genapol 1261®, and clove oil.

Another embodiment comprises cyclosporin A, Generol 122 E25®, and clove oil.

Another embodiment comprises cyclosporin A, Glicepol 160®, and clove oil.

Another embodiment comprises cyclosporin A, Glicepol 560®, and clove oil.

Another embodiment comprises cyclosporin A, Glicepol GMS 20®, and clove oil.

Another embodiment comprises cyclosporin A, Hetoxide BN-13®, and clove oil.

Another embodiment comprises cyclosporin A, Hetoxide DNP-4®, and clove oil.

Another embodiment comprises cyclosporin A, Hetoxide DNP-9.6®, and clove oil.

Another embodiment comprises cyclosporin A, Hetoxide G-26®, and clove oil.

Another embodiment comprises cyclosporin A, Hetoxide G-7®, and clove oil.

Another embodiment comprises cyclosporin A, Hetoxide GT-20®, and clove oil.

Another embodiment comprises cyclosporin A, Hetoxide HC-40®, and clove oil.

Another embodiment comprises cyclosporin A, Hetoxol C-24®, and clove oil.

Another embodiment comprises cyclosporin A, Hetoxol CA-10®, and clove oil.

Another embodiment comprises cyclosporin A, Hetoxol CA-2®, and clove oil.

Another embodiment comprises cyclosporin A, Hetoxol CA-20®, and clove oil.

Another embodiment comprises cyclosporin A, Hetoxol CAWS®, and clove oil.

Another embodiment comprises cyclosporin A, Hetoxol CD-3®, and clove oil.

Another embodiment comprises cyclosporin A, Hetoxol CD-4®, and clove oil.

Another embodiment comprises cyclosporin A, Hetoxol CS-15®, and clove oil.

Another embodiment comprises cyclosporin A, Hetoxol CS-20®, and clove oil.

Another embodiment comprises cyclosporin A, Hetoxol CS-20D®, and clove oil.

Another embodiment comprises cyclosporin A, Hetoxol CS-25®, and clove oil.

Another embodiment comprises cyclosporin A, Hetoxol CS-50®, and clove oil.

Another embodiment comprises cyclosporin A, Hetoxol CS-9®, and clove oil.

Another embodiment comprises cyclosporin A, Hetoxol CSA-15®, and clove oil.

Another embodiment comprises cyclosporin A, Hetoxol D®, and clove oil.

Another embodiment comprises cyclosporin A, Hetoxol G®, and clove oil.

Another embodiment comprises cyclosporin A, Hetoxol J®, and clove oil.

Another embodiment comprises cyclosporin A, Hetoxol L®, and clove oil.

Another embodiment comprises cyclosporin A, Hetoxol L-23®, and clove oil.

Another embodiment comprises cyclosporin A, Hetoxol L-4®, and clove oil.

Another embodiment comprises cyclosporin A, Hetoxol L-9®, and clove oil.

Another embodiment comprises cyclosporin A, Hetoxol LS-9®, and clove oil.

Another embodiment comprises cyclosporin A, Hetoxol M-3®, and clove oil.

Another embodiment comprises cyclosporin A, Hetoxol OA-10 Special®, and clove oil.

Another embodiment comprises cyclosporin A, Hetoxol OA-20 Special®, and clove oil.

Another embodiment comprises cyclosporin A, Hetoxol OA-3 Special®, and clove oil.

Another embodiment comprises cyclosporin A, Hetoxol OA-5 Special®, and clove oil.

Another embodiment comprises cyclosporin A, Hetoxol OL-10®, and clove oil.

Another embodiment comprises cyclosporin A, Hetoxol OL-2®, and clove oil.

Another embodiment comprises cyclosporin A, Hetoxol OL-23®, and clove oil.

Another embodiment comprises cyclosporin A, Hetoxol OL-4®, and clove oil.

Another embodiment comprises cyclosporin A, Hetoxol OL-40®, and clove oil.

Another embodiment comprises cyclosporin A, Hetoxol OL-5®, and clove oil.

Another embodiment comprises cyclosporin A, Hetoxol PLA®, and clove oil.

Another embodiment comprises cyclosporin A, Hetoxol SP-15®, and clove oil.

Another embodiment comprises cyclosporin A, Hetoxol STA-10®, and clove oil.

Another embodiment comprises cyclosporin A, Hetoxol STA-2®, and clove oil.

Another embodiment comprises cyclosporin A, Hetoxol STA-30®, and clove oil.

Another embodiment comprises cyclosporin A, Hetoxol TD-12®, and clove oil.

Another embodiment comprises cyclosporin A, Hetoxol TD-18®, and clove oil.

Another embodiment comprises cyclosporin A, Hetoxol TD-3®, and clove oil.

Another embodiment comprises cyclosporin A, Hetoxol TD-6®, and clove oil.

Another embodiment comprises cyclosporin A, Hetoxol TD-9®, and clove oil.

Another embodiment comprises cyclosporin A, Hetoxol TDEP-15®, and clove oil.

Another embodiment comprises cyclosporin A, Hetoxol TDEP-63®, and clove oil.

Another embodiment comprises cyclosporin A, Iconol 24-12®, and clove oil.

Another embodiment comprises cyclosporin A, Iconol 24-23®, and clove oil.

Another embodiment comprises cyclosporin A, Iconol 24-4®, and clove oil.

Another embodiment comprises cyclosporin A, Iconol DA-4®, and clove oil.

Another embodiment comprises cyclosporin A, Iconol DA-6®, and clove oil.

Another embodiment comprises cyclosporin A, Iconol TDA-10®, and clove oil.

Another embodiment comprises cyclosporin A, Iconol TDA-3®, and clove oil.

Another embodiment comprises cyclosporin A, Iconol TDA-6®, and clove oil.

Another embodiment comprises cyclosporin A, Iconol TDA-8-90%®, and clove oil.

Another embodiment comprises cyclosporin A, Iconol TDA-9®, and clove oil.

Another embodiment comprises cyclosporin A, Igepal CA-210®, and clove oil.

Another embodiment comprises cyclosporin A, Igepal CA-407®, and clove oil.

Another embodiment comprises cyclosporin A, Igepal CA-420®, and clove oil.

Another embodiment comprises cyclosporin A, Igepal CA-520®, and clove oil.

Another embodiment comprises cyclosporin A, Igepal CA-620®, and clove oil.

Another embodiment comprises cyclosporin A, Igepal CA-630®, and clove oil.

Another embodiment comprises cyclosporin A, Igepal CA-720®, and clove oil.

Another embodiment comprises cyclosporin A, Igepal CA-877®, and clove oil.

Another embodiment comprises cyclosporin A, Igepal CA-887®, and clove oil.

Another embodiment comprises cyclosporin A, Igepal CA-897®, and clove oil.

Another embodiment comprises cyclosporin A, Igepal CO-210®, and clove oil.

Another embodiment comprises cyclosporin A, Igepal CO-430®, and clove oil.

Another embodiment comprises cyclosporin A, Igepal CO-520®, and clove oil.

Another embodiment comprises cyclosporin A, Igepal CO-530®, and clove oil.

Another embodiment comprises cyclosporin A, Igepal CO-610®, and clove oil.

Another embodiment comprises cyclosporin A, Igepal CO-630®, and clove oil.

Another embodiment comprises cyclosporin A, Igepal CO-630 Special®, and clove oil.

Another embodiment comprises cyclosporin A, Igepal CO-660®, and clove oil.

Another embodiment comprises cyclosporin A, Igepal CO-710®, and clove oil.

Another embodiment comprises cyclosporin A, Igepal CO-720®, and clove oil.

Another embodiment comprises cyclosporin A, Igepal CO-730®, and clove oil.

Another embodiment comprises cyclosporin A, Igepal CO-738®, and clove oil.

Another embodiment comprises cyclosporin A, Igepal CO-850®, and clove oil.

Another embodiment comprises cyclosporin A, Igepal CO-880®, and clove oil.

Another embodiment comprises cyclosporin A, Igepal CO-887®, and clove oil.

Another embodiment comprises cyclosporin A, Igepal CO-890®, and clove oil.

Another embodiment comprises cyclosporin A, Igepal CO-897®, and clove oil.

Another embodiment comprises cyclosporin A, Igepal CO-970®, and clove oil.

Another embodiment comprises cyclosporin A, Igepal CO-977®, and clove oil.

Another embodiment comprises cyclosporin A, Igepal CO-987®, and clove oil.

Another embodiment comprises cyclosporin A, Igepal CO-990 FLK®, and clove oil.

Another embodiment comprises cyclosporin A, Igepal CO-997®, and clove oil.

Another embodiment comprises cyclosporin A, Igepal DM-430®, and clove oil.

Another embodiment comprises cyclosporin A, Igepal DM-530®, and clove oil.

Another embodiment comprises cyclosporin A, Igepal DM-710®, and clove oil.

Another embodiment comprises cyclosporin A, Igepal DM-730®, and clove oil.

Another embodiment comprises cyclosporin A, Igepal DM-880®, and clove oil.

Another embodiment comprises cyclosporin A, Igepal DM-970 FLK®, and clove oil.

Another embodiment comprises cyclosporin A, Igepal OD-410®, and clove oil.

Another embodiment comprises cyclosporin A, Igepal RC-520®, and clove oil.

Another embodiment comprises cyclosporin A, Igepal RC-620®, and clove oil.

Another embodiment comprises cyclosporin A, Igepal RC-630®, and clove oil.

Another embodiment comprises cyclosporin A, Lexemul CS-20®, and clove oil.

Another embodiment comprises cyclosporin A, Lipo GMS 450®, and clove oil.

Another embodiment comprises cyclosporin A, Lipo Polyglycol 1000®, and clove oil.

Another embodiment comprises cyclosporin A, Lipo Polyglycol 200®, and clove oil.

Another embodiment comprises cyclosporin A, Lipo Polyglycol 300®, and clove oil.

Another embodiment comprises cyclosporin A, Lipo Polyglycol 3350®, and clove oil.

Another embodiment comprises cyclosporin A, Lipo Polyglycol 400®, and clove oil.

Another embodiment comprises cyclosporin A, Lipo Polyglycol 600®, and clove oil.

Another embodiment comprises cyclosporin A, Lipocol C-10®, and clove oil.

Another embodiment comprises cyclosporin A, Lipocol C-2®, and clove oil.

Another embodiment comprises cyclosporin A, Lipocol C-20®, and clove oil.

Another embodiment comprises cyclosporin A, Lipocol L-12®, and clove oil.

Another embodiment comprises cyclosporin A, Lipocol L-23®, and clove oil.

Another embodiment comprises cyclosporin A, Lipocol L-4®, and clove oil.

Another embodiment comprises cyclosporin A, Lipocol O-10®, and clove oil.

Another embodiment comprises cyclosporin A, Lipocol O-2®, and clove oil.

Another embodiment comprises cyclosporin A, Lipocol O-20®, and clove oil.

Another embodiment comprises cyclosporin A, Lipocol O-50, and clove oil.

Another embodiment comprises cyclosporin A, Lipocol S-10®, and clove oil.

Another embodiment comprises cyclosporin A, Lipocol S-2®, and clove oil.

Another embodiment comprises cyclosporin A, Lipocol S-20®, and clove oil.

Another embodiment comprises cyclosporin A, Lipocol SC-15®, and clove oil.

Another embodiment comprises cyclosporin A, Lipocol SC-20®, and clove oil.

Another embodiment comprises cyclosporin A, Lipocol SC-4®, and clove oil.

Another embodiment comprises cyclosporin A, Lipocol TD-12®, and clove oil.

Another embodiment comprises cyclosporin A, Lumulse 40-O K®, and clove oil.

Another embodiment comprises cyclosporin A, Lumulse 42-O K®, and clove oil.

Another embodiment comprises cyclosporin A, Lumulse GMT-40®, and clove oil.

Another embodiment comprises cyclosporin A, Lumulse PEG 1450®, and clove oil.

Another embodiment comprises cyclosporin A, Lumulse PEG 1450 NF®, and clove oil.

Another embodiment comprises cyclosporin A, Lumulse PEG 8000®, and clove oil.

Another embodiment comprises cyclosporin A, Lumulse PGO®, and clove oil.

Another embodiment comprises cyclosporin A, Lumulse POE (2) Oleyl Amine®, and clove oil.

Another embodiment comprises cyclosporin A, Magrabar PGO-104®, and clove oil.

Another embodiment comprises cyclosporin A, Mapeg 200 ML®, and clove oil.

Another embodiment comprises cyclosporin A, Mapeg 400 MO®, and clove oil.

Another embodiment comprises cyclosporin A, Mapeg 400 MOT®, and clove oil.

Another embodiment comprises cyclosporin A, Mapeg 400DO®, and clove oil.

Another embodiment comprises cyclosporin A, Mapeg 400DOT®, and clove oil.

Another embodiment comprises cyclosporin A, Mapeg 600DOT®, and clove oil.

Another embodiment comprises cyclosporin A, Marlox B 24/80®, and clove oil.

Another embodiment comprises cyclosporin A, Marlox FK 86®, and clove oil.

Another embodiment comprises cyclosporin A, Marlox MO 154®, and clove oil.

Another embodiment comprises cyclosporin A, Mazox CDA CG®, and clove oil.

Another embodiment comprises cyclosporin A, Monoglycerides Glyceryl Monestearate®, and clove oil.

Another embodiment comprises cyclosporin A, Myrj 45®, and clove oil.

Another embodiment comprises cyclosporin A, Myrj 52®, and clove oil.

Another embodiment comprises cyclosporin A, Myrj 52S®, and clove oil.

Another embodiment comprises cyclosporin A, Myrj 53®, and clove oil.

Another embodiment comprises cyclosporin A, Myrj 59®, and clove oil.

Another embodiment comprises cyclosporin A, Norfox NP-12®, and clove oil.

Another embodiment comprises cyclosporin A, Norfox NP-4®, and clove oil.

Another embodiment comprises cyclosporin A, Norfox NP-6®, and clove oil.

Another embodiment comprises cyclosporin A, Norfox NP-887®, and clove oil.

Another embodiment comprises cyclosporin A, Norfox NP-9®, and clove oil.

Another embodiment comprises cyclosporin A, Norfox NP-977®, and clove oil.

Another embodiment comprises cyclosporin A, Norfox OP-100®, and clove oil.

Another embodiment comprises cyclosporin A, Norfox OP-102®, and clove oil.

Another embodiment comprises cyclosporin A, Norfox OP-114®, and clove oil.

Another embodiment comprises cyclosporin A, Norfox OP-45®, and clove oil.

Another embodiment comprises cyclosporin A, Pegosperse 100-L®, and clove oil.

Another embodiment comprises cyclosporin A, Pegosperse 100-O®, and clove oil.

Another embodiment comprises cyclosporin A, Pegosperse 100-S®, and clove oil.

Another embodiment comprises cyclosporin A, Pegosperse 1500-MS®, and clove oil.

Another embodiment comprises cyclosporin A, Pegosperse 1750-MS®, and clove oil.

Another embodiment comprises cyclosporin A, Pegosperse 200 DL®, and clove oil.

Another embodiment comprises cyclosporin A, Pegosperse 200-ML®, and clove oil.

Another embodiment comprises cyclosporin A, Pegosperse 400-DL®, and clove oil.

Another embodiment comprises cyclosporin A, Pegosperse 400-DO®, and clove oil.

Another embodiment comprises cyclosporin A, Pegosperse 400-DS®, and clove oil.

Another embodiment comprises cyclosporin A, Pegosperse 400-ML®, and clove oil.

Another embodiment comprises cyclosporin A, Pegosperse 400-MO®, and clove oil.

Another embodiment comprises cyclosporin A, Pegosperse 400-MOT®, and clove oil.

Another embodiment comprises cyclosporin A, Pegosperse 400-MS®, and clove oil.

Another embodiment comprises cyclosporin A, Pegosperse 50-DS®, and clove oil.

Another embodiment comprises cyclosporin A, Pegosperse 50-MS®, and clove oil.

Another embodiment comprises cyclosporin A, Pegosperse 600-ML®, and clove oil.

Another embodiment comprises cyclosporin A, Pegosperse 600-MS®, and clove oil.

Another embodiment comprises cyclosporin A, Plurafac A-38®, and clove oil.

Another embodiment comprises cyclosporin A, Plurafac B-25-5®, and clove oil.

Another embodiment comprises cyclosporin A, Plurafac B-26®, and clove oil.

Another embodiment comprises cyclosporin A, Plurafac D-25®, and clove oil.

Another embodiment comprises cyclosporin A, Plurafac LF 131®, and clove oil.

Another embodiment comprises cyclosporin A, Plurafac LF 4030®, and clove oil.

Another embodiment comprises cyclosporin A, Plurafac LF 7000®, and clove oil.

Another embodiment comprises cyclosporin A, Plurafac RA-20®, and clove oil.

Another embodiment comprises cyclosporin A, Plurafac RA-30®, and clove oil.

Another embodiment comprises cyclosporin A, Plurafac RA-40®, and clove oil.

Another embodiment comprises cyclosporin A, Pluronic 10R5®, and clove oil.

Another embodiment comprises cyclosporin A, Pluronic 17R2®, and clove oil.

Another embodiment comprises cyclosporin A, Pluronic 17R4®, and clove oil.

Another embodiment comprises cyclosporin A, Pluronic 25R2®, and clove oil.

Another embodiment comprises cyclosporin A, Pluronic 25R4®, and clove oil.

Another embodiment comprises cyclosporin A, Pluronic 31R1®, and clove oil.

Another embodiment comprises cyclosporin A, Pluronic F108®, and clove oil.

Another embodiment comprises cyclosporin A, Pluronic F127®, and clove oil.

Another embodiment comprises cyclosporin A, Pluronic F38®, and clove oil.

Another embodiment comprises cyclosporin A, Pluronic F68®, and clove oil.

Another embodiment comprises cyclosporin A, Pluronic F68LF®, and clove oil.

Another embodiment comprises cyclosporin A, Pluronic F77®, and clove oil.

Another embodiment comprises cyclosporin A, Pluronic F87®, and clove oil.

Another embodiment comprises cyclosporin A, Pluronic F88®, and clove oil.

Another embodiment comprises cyclosporin A, Pluronic F98®, and clove oil.

Another embodiment comprises cyclosporin A, Pluronic L10®, and clove oil.

Another embodiment comprises cyclosporin A, Pluronic L101®, and clove oil.

Another embodiment comprises cyclosporin A, Pluronic L121®, and clove oil.

Another embodiment comprises cyclosporin A, Pluronic L31®, and clove oil.

Another embodiment comprises cyclosporin A, Pluronic L35®, and clove oil.

Another embodiment comprises cyclosporin A, Pluronic L43®, and clove oil.

Another embodiment comprises cyclosporin A, Pluronic L44®, and clove oil.

Another embodiment comprises cyclosporin A, Pluronic L61®, and clove oil.

Another embodiment comprises cyclosporin A, Pluronic L62®, and clove oil.

Another embodiment comprises cyclosporin A, Pluronic L62D®, and clove oil.

Another embodiment comprises cyclosporin A, Pluronic L62LF®, and clove oil.

Another embodiment comprises cyclosporin A, Pluronic L64®, and clove oil.

Another embodiment comprises cyclosporin A, Pluronic L81®, and clove oil.

Another embodiment comprises cyclosporin A, Pluronic L92®, and clove oil.

Another embodiment comprises cyclosporin A, Pluronic P103®, and clove oil.

Another embodiment comprises cyclosporin A, Pluronic P104®, and clove oil.

Another embodiment comprises cyclosporin A, Pluronic P105®, and clove oil.

Another embodiment comprises cyclosporin A, Pluronic P123®, and clove oil.

Another embodiment comprises cyclosporin A, Pluronic P65®, and clove oil.

Another embodiment comprises cyclosporin A, Pluronic P84®, and clove oil.

Another embodiment comprises cyclosporin A, Pluronic P85®, and clove oil.

Another embodiment comprises cyclosporin A, Polyaldo 10-2-P®, and clove oil.

Another embodiment comprises cyclosporin A, Polyaldo DGDO®, and clove oil.

Another embodiment comprises cyclosporin A, Polyaldo HGDS®, and clove oil.

Another embodiment comprises cyclosporin A, Polyaldo TGMS®, and clove oil.

Another embodiment comprises cyclosporin A, Protegin V®, and clove oil.

Another embodiment comprises cyclosporin A, Protegin W®, and clove oil.

Another embodiment comprises cyclosporin A, Protegin WX®, and clove oil.

Another embodiment comprises cyclosporin A, Protegin XV®, and clove oil.

Another embodiment comprises cyclosporin A, Rhodasurf BC-420®, and clove oil.

Another embodiment comprises cyclosporin A, Rhodasurf BC-610®, and clove oil.

Another embodiment comprises cyclosporin A, Rhodasurf BC-630®, and clove oil.

Another embodiment comprises cyclosporin A, Rhodasurf BC-720®, and clove oil.

Another embodiment comprises cyclosporin A, Rhodasurf BC-840®, and clove oil.

Another embodiment comprises cyclosporin A, Rhodasurf L 1®, and clove oil.

Another embodiment comprises cyclosporin A, Rhodasurf L 12®, and clove oil.

Another embodiment comprises cyclosporin A, Rhodasurf L 2®, and clove oil.

Another embodiment comprises cyclosporin A, Rhodasurf L 20®, and clove oil.

Another embodiment comprises cyclosporin A, Rhodasurf L 3®, and clove oil.

Another embodiment comprises cyclosporin A, Rhodasurf L 30®, and clove oil.

Another embodiment comprises cyclosporin A, Rhodasurf L 4®, and clove oil.

Another embodiment comprises cyclosporin A, Rhodasurf L 50®, and clove oil.

Another embodiment comprises cyclosporin A, Rhodasurf L 7/90®, and clove oil.

Another embodiment comprises cyclosporin A, Rhodasurf LA-3®, and clove oil.

Another embodiment comprises cyclosporin A, Rhodasurf LA-7®, and clove oil.

Another embodiment comprises cyclosporin A, Rhodasurf LAN-23®, and clove oil.

Another embodiment comprises cyclosporin A, Rhodasurf ON-870®, and clove oil.

Another embodiment comprises cyclosporin A, Rhodasurf ON-877®, and clove oil.

Another embodiment comprises cyclosporin A, Rhodasurf TDA-8/5®, and clove oil.

Another embodiment comprises cyclosporin A, Ritapro 100®, and clove oil.

Another embodiment comprises cyclosporin A, Ritapro 165®, and clove oil.

Another embodiment comprises cyclosporin A, Ritapro 200®, and clove oil.

Another embodiment comprises cyclosporin A, Ritapro 300®, and clove oil.

Another embodiment comprises cyclosporin A, Ritoleth 10®, and clove oil.

Another embodiment comprises cyclosporin A, Ritoleth 2®, and clove oil.

Another embodiment comprises cyclosporin A, Ritoleth 20®, and clove oil.

Another embodiment comprises cyclosporin A, Ritoleth 5®, and clove oil.

Another embodiment comprises cyclosporin A, Santone 10-10-0®, and clove oil.

Another embodiment comprises cyclosporin A, Santone 3-1-S XTR®, and clove oil.

Another embodiment comprises cyclosporin A, Santone 3-1-SH®, and clove oil.

Another embodiment comprises cyclosporin A, Santone 8-1-O®, and clove oil.

Another embodiment comprises cyclosporin A, Solulan 16®, and clove oil.

Another embodiment comprises cyclosporin A, Solulan 75®, and clove oil.

Another embodiment comprises cyclosporin A, Solulan C-24®, and clove oil.

Another embodiment comprises cyclosporin A, Solulan L-575®, and clove oil.

Another embodiment comprises cyclosporin A, Standamox CAW®, and clove oil.

Another embodiment comprises cyclosporin A, Surfine AZI-A®, and clove oil.

Another embodiment comprises cyclosporin A, Surfonic CO-15®, and clove oil.

Another embodiment comprises cyclosporin A, Surfonic CO-25®, and clove oil.

Another embodiment comprises cyclosporin A, Surfonic CO-30®, and clove oil.

Another embodiment comprises cyclosporin A, Surfonic CO-36®, and clove oil.

Another embodiment comprises cyclosporin A, Surfonic CO-42®, and clove oil.

Another embodiment comprises cyclosporin A, Surfonic E400-MO®, and clove oil.

Another embodiment comprises cyclosporin A, Surfonic L12-3®, and clove oil.

Another embodiment comprises cyclosporin A, Surfonic L12-6®, and clove oil.

Another embodiment comprises cyclosporin A, Surfonic L12-8®, and clove oil.

Another embodiment comprises cyclosporin A, Surfonic L24-12®, and clove oil.

Another embodiment comprises cyclosporin A, Surfonic L24-2®, and clove oil.

Another embodiment comprises cyclosporin A, Surfonic L24-22®, and clove oil.

Another embodiment comprises cyclosporin A, Surfonic L24-3®, and clove oil.

Another embodiment comprises cyclosporin A, Surfonic L24-4®, and clove oil.

Another embodiment comprises cyclosporin A, Surfonic L24-5®, and clove oil.

Another embodiment comprises cyclosporin A, Surfonic L24-7®, and clove oil.

Another embodiment comprises cyclosporin A, Surfonic L24-7.1®, and clove oil.

Another embodiment comprises cyclosporin A, Surfonic L24-9®, and clove oil.

Another embodiment comprises cyclosporin A, Surfonic LF-17®, and clove oil.

Another embodiment comprises cyclosporin A, Surfonic LF-18®, and clove oil.

Another embodiment comprises cyclosporin A, Surfonic LF-37®, and clove oil.

Another embodiment comprises cyclosporin A, Surfonic LF-41®, and clove oil.

Another embodiment comprises cyclosporin A, Surfonic LF-42®, and clove oil.

Another embodiment comprises cyclosporin A, Surfonic LF-47®, and clove oil.

Another embodiment comprises cyclosporin A, Surfonic LF-68®, and clove oil.

Another embodiment comprises cyclosporin A, Surfonic N-10®, and clove oil.

Another embodiment comprises cyclosporin A, Surfonic N-100®, and clove oil.

Another embodiment comprises cyclosporin A, Surfonic N-102®, and clove oil.

Another embodiment comprises cyclosporin A, Surfonic N-120®, and clove oil.

Another embodiment comprises cyclosporin A, Surfonic N-150®, and clove oil.

Another embodiment comprises cyclosporin A, Surfonic N-200®, and clove oil.

Another embodiment comprises cyclosporin A, Surfonic N-300®, and clove oil.

Another embodiment comprises cyclosporin A, Surfonic N-31.5®, and clove oil.

Another embodiment comprises cyclosporin A, Surfonic N-40®, and clove oil.

Another embodiment comprises cyclosporin A, Surfonic N-400®, and clove oil.

Another embodiment comprises cyclosporin A, Surfonic N-60®, and clove oil.

Another embodiment comprises cyclosporin A, Surfonic N-85®, and clove oil.

Another embodiment comprises cyclosporin A, Surfonic N-95®, and clove oil.

Another embodiment comprises cyclosporin A, Surfonic NB-18®, and clove oil.

Another embodiment comprises cyclosporin A, Surfonic NB-23®, and clove oil.

Another embodiment comprises cyclosporin A, Surfonic NB-25®, and clove oil.

Another embodiment comprises cyclosporin A, Surfonic NB-307®, and clove oil.

Another embodiment comprises cyclosporin A, Surfonic NB-31®, and clove oil.

Another embodiment comprises cyclosporin A, Surfonic NB-41®, and clove oil.

Another embodiment comprises cyclosporin A, Surfonic NB-43®, and clove oil.

Another embodiment comprises cyclosporin A, Surfonic OP-100®, and clove oil.

Another embodiment comprises cyclosporin A, Surfonic OP-120®, and clove oil.

Another embodiment comprises cyclosporin A, Surlonic OP-15®, and clove oil.

Another embodiment comprises cyclosporin A, Surfonic OP-35®, and clove oil.

Another embodiment comprises cyclosporin A, Surfonic OP-70®, and clove oil.

Another embodiment comprises cyclosporin A, Surfonic P1®, and clove oil.

Another embodiment comprises cyclosporin A, Surfonic P3®, and clove oil.

Another embodiment comprises cyclosporin A, Surfonic P5®, and clove oil.

Another embodiment comprises cyclosporin A, Surfonic P6®, and clove oil.

Another embodiment comprises cyclosporin A, Surfonic POA-17R2®, and clove oil.

Another embodiment comprises cyclosporin A, Surfonic POA-25R2®, and clove oil.

Another embodiment comprises cyclosporin A, Surfonic TDA-11®, and clove oil.

Another embodiment comprises cyclosporin A, Surfonic TDA-3B®, and clove oil.

Another embodiment comprises cyclosporin A, Surfonic TDA-6®, and clove oil.

Another embodiment comprises cyclosporin A, Surfonic TDA-6/88®, and clove oil.

Another embodiment comprises cyclosporin A, Surfonic TDA-8®, and clove oil.

Another embodiment comprises cyclosporin A, Surfonic TDA-8/90®, and clove oil.

Another embodiment comprises cyclosporin A, Surfonic TDA-9®, and clove oil.

Another embodiment comprises cyclosporin A, Surfonic TX-CCN®, and clove oil.

Another embodiment comprises cyclosporin A, Tagat CH 40®, and clove oil.

Another embodiment comprises cyclosporin A, Tagat CH 60®, and clove oil.

Another embodiment comprises cyclosporin A, Tagat L 2®, and clove oil.

Another embodiment comprises cyclosporin A, Tagat S®, and clove oil.

Another embodiment comprises cyclosporin A, Tagat S 2®, and clove oil.

Another embodiment comprises cyclosporin A, Tally 100 Plus®, and clove oil.

Another embodiment comprises cyclosporin A, T-Det A106®, and clove oil.

Another embodiment comprises cyclosporin A, T-Det A109®, and clove oil.

Another embodiment comprises cyclosporin A, T-Det A136®, and clove oil.

Another embodiment comprises cyclosporin A, T-Det A139®, and clove oil.

Another embodiment comprises cyclosporin A, T-Det A2412®, and clove oil.

Another embodiment comprises cyclosporin A, T-Det A243®, and clove oil.

Another embodiment comprises cyclosporin A, T-Det A247®, and clove oil.

Another embodiment comprises cyclosporin A, T-Det A249®, and clove oil.

Another embodiment comprises cyclosporin A, T-Det A467®, and clove oil.

Another embodiment comprises cyclosporin A, T-Det COE®, and clove oil.

Another embodiment comprises cyclosporin A, T-Det DD-10®, and clove oil.

Another embodiment comprises cyclosporin A, T-Det DD-5®, and clove oil.

Another embodiment comprises cyclosporin A, T-Det DD-7®, and clove oil.

Another embodiment comprises cyclosporin A, T-Det EPO-104®, and clove oil.

Another embodiment comprises cyclosporin A, T-Det EPO-61®, and clove oil.

Another embodiment comprises cyclosporin A, T-Det EPO-62®, and clove oil.

Another embodiment comprises cyclosporin A, T-Det EPO-64®, and clove oil.

Another embodiment comprises cyclosporin A, T-Det N-1.5®, and clove oil.

Another embodiment comprises cyclosporin A, T-Det N-10.5®, and clove oil.

Another embodiment comprises cyclosporin A, T-Det N-100®, and clove oil.

Another embodiment comprises cyclosporin A, T-Det N-1007®, and clove oil.

Another embodiment comprises cyclosporin A, T-Det N-12®, and clove oil.

Another embodiment comprises cyclosporin A, T-Det N-14®, and clove oil.

Another embodiment comprises cyclosporin A, T-Det N-20®, and clove oil.

Another embodiment comprises cyclosporin A, T-Det N-30®, and clove oil.

Another embodiment comprises cyclosporin A, T-Det N-307®, and clove oil.

Another embodiment comprises cyclosporin A, T-Det N-4®, and clove oil.

Another embodiment comprises cyclosporin A, T-Det N-40®, and clove oil.

Another embodiment comprises cyclosporin A, T-Det N-407®, and clove oil.

Another embodiment comprises cyclosporin A, T-Det N-50®, and clove oil.

Another embodiment comprises cyclosporin A, T-Det N-507®, and clove oil.

Another embodiment comprises cyclosporin A, T-Det N-6®, and clove oil.

Another embodiment comprises cyclosporin A, T-Det N-70®, and clove oil.

Another embodiment comprises cyclosporin A, T-Det N-8®, and clove oil.

Another embodiment comprises cyclosporin A, T-Det N-9.5®, and clove oil.

Another embodiment comprises cyclosporin A, T-Det O-12®, and clove oil.

Another embodiment comprises cyclosporin A, T-Det O-165®, and clove oil.

Another embodiment comprises cyclosporin A, T-Det O-307®, and clove oil.

Another embodiment comprises cyclosporin A, T-Det O-40®, and clove oil.

Another embodiment comprises cyclosporin A, T-Det O-407®, and clove oil.

Another embodiment comprises cyclosporin A, T-Det O-6®, and clove oil.

Another embodiment comprises cyclosporin A, T-Det O-8®, and clove oil.

Another embodiment comprises cyclosporin A, T-Det O-9®, and clove oil.

Another embodiment comprises cyclosporin A, Tegin®, and clove oil.

Another embodiment comprises cyclosporin A, Tergitol 15-S-12®, and clove oil.

Another embodiment comprises cyclosporin A, Tergitol 15-S-15®, and clove oil.

Another embodiment comprises cyclosporin A, Tergitol 15-S-20®, and clove oil.

Another embodiment comprises cyclosporin A, Tergitol 15-S-20 (80AQ)®, and clove oil.

Another embodiment comprises cyclosporin A, Tergitol 15-S-3®, and clove oil.

Another embodiment comprises cyclosporin A, Tergitol 15-S-30®, and clove oil.

Another embodiment comprises cyclosporin A, Tergitol 15-S-40®, and clove oil.

Another embodiment comprises cyclosporin A, Tergitol 15-S-40 (70AQ)®, and clove oil.

Another embodiment comprises cyclosporin A, Tergitol 15-S-5®, and clove oil.

Another embodiment comprises cyclosporin A, Tergitol 15-S-7®, and clove oil.

Another embodiment comprises cyclosporin A, Tergitol 15-S-9®, and clove oil.

Another embodiment comprises cyclosporin A, Tergitol L-101®, and clove oil.

Another embodiment comprises cyclosporin A, Tergitol L-61®, and clove oil.

Another embodiment comprises cyclosporin A, Tergitol L-62®, and clove oil.

Another embodiment comprises cyclosporin A, Tergitol L-64®, and clove oil.

Another embodiment comprises cyclosporin A, Tergitol L-81®, and clove oil.

Another embodiment comprises cyclosporin A, Tergitol NP-10®, and clove oil.

Another embodiment comprises cyclosporin A, Tergitol NP-11®, and clove oil.

Another embodiment comprises cyclosporin A, Tergitol NP-12®, and clove oil.

Another embodiment comprises cyclosporin A, Tergitol NP-13®, and clove oil.

Another embodiment comprises cyclosporin A, Tergitol NP-15®, and clove oil.

Another embodiment comprises cyclosporin A, Tergitol NP-30®, and clove oil.

Another embodiment comprises cyclosporin A, Tergitol NP-30 (70 AQ)®, and clove oil.

Another embodiment comprises cyclosporin A, Tergitol NP-4®, and clove oil.

Another embodiment comprises cyclosporin A, Tergitol NP-40®, and clove oil.

Another embodiment comprises cyclosporin A, Tergitol NP-40 (70 AQ)®, and clove oil.

Another embodiment comprises cyclosporin A, Tergitol NP-50 (70 AQ)®, and clove oil.

Another embodiment comprises cyclosporin A, Tergitol NP-55 (70 AQ)®, and clove oil.

Another embodiment comprises cyclosporin A, Tergitol NP-6®, and clove oil.

Another embodiment comprises cyclosporin A, Tergitol NP-7®, and clove oil.

Another embodiment comprises cyclosporin A, Tergitol NP-70 (70 AQ)®, and clove oil.

Another embodiment comprises cyclosporin A, Tergitol NP-8®, and clove oil.

Another embodiment comprises cyclosporin A, Tergitol NP-9®, and clove oil.

Another embodiment comprises cyclosporin A, Tergitol NP-9.5®, and clove oil.

Another embodiment comprises cyclosporin A, Tergitol TMN-10®, and clove oil.

Another embodiment comprises cyclosporin A, Tergitol TMN-3®, and clove oil.

Another embodiment comprises cyclosporin A, Tergitol TMN-6®, and clove oil.

Another embodiment comprises cyclosporin A, Tergitol XD®, and clove oil.

Another embodiment comprises cyclosporin A, Tergitol XH®, and clove oil.

Another embodiment comprises cyclosporin A, Tergitol XJ®, and clove oil.

Another embodiment comprises cyclosporin A, Tetronic 1107®, and clove oil.

Another embodiment comprises cyclosporin A, Tetronic 1307®, and clove oil.

Another embodiment comprises cyclosporin A, Tetronic 150R1®, and clove oil.

Another embodiment comprises cyclosporin A, Tetronic 304®, and clove oil.

Another embodiment comprises cyclosporin A, Tetronic 701®, and clove oil.

Another embodiment comprises cyclosporin A, Tetronic 901®, and clove oil.

Another embodiment comprises cyclosporin A, Tetronic 904®, and clove oil.

Another embodiment comprises cyclosporin A, Tetronic 908®, and clove oil.

Another embodiment comprises cyclosporin A, Tetronic 90R4®, and clove oil.

Another embodiment comprises cyclosporin A, Tomadol 1-3®, and clove oil.

Another embodiment comprises cyclosporin A, Tomadol 1-5®, and clove oil.

Another embodiment comprises cyclosporin A, Tomadol 1-7®, and clove oil.

Another embodiment comprises cyclosporin A, Tomadol 23-1®, and clove oil.

Another embodiment comprises cyclosporin A, Tomadol 23-3®, and clove oil.

Another embodiment comprises cyclosporin A, Tomadol 23-5®, and clove oil.

Another embodiment comprises cyclosporin A, Tomadol 23-6.5®, and clove oil.

Another embodiment comprises cyclosporin A, Tomadol 25-12®, and clove oil.

Another embodiment comprises cyclosporin A, Tomadol 25-3®, and clove oil.

Another embodiment comprises cyclosporin A, Tomadol 25-7®, and clove oil.

Another embodiment comprises cyclosporin A, Tomadol 25-9®, and clove oil.

Another embodiment comprises cyclosporin A, Tomadol 45-13®, and clove oil.

Another embodiment comprises cyclosporin A, Tomadol 45-2.5®, and clove oil.

Another embodiment comprises cyclosporin A, Tomadol 45-7®, and clove oil.

Another embodiment comprises cyclosporin A, Tomadol 91-2.5®, and clove oil.

Another embodiment comprises cyclosporin A, Tomadol 91-6®, and clove oil.

Another embodiment comprises cyclosporin A, Tomadol 91-8®, and clove oil.

Another embodiment comprises cyclosporin A, Triton X-100®, and clove oil.

Another embodiment comprises cyclosporin A, Triton X-102®, and clove oil.

Another embodiment comprises cyclosporin A, Triton X-114®, and clove oil.

Another embodiment comprises cyclosporin A, Triton X-15®, and clove oil.

Another embodiment comprises cyclosporin A, Triton X-165 (70%)®, and clove oil.

Another embodiment comprises cyclosporin A, Triton X-207®, and clove oil.

Another embodiment comprises cyclosporin A, Triton X-305 (70%)®, and clove oil.

Another embodiment comprises cyclosporin A, Triton X-35®, and clove oil.

Another embodiment comprises cyclosporin A, Triton X-405 (70%)®, and clove oil.

Another embodiment comprises cyclosporin A, Triton X-45®, and clove oil.

Another embodiment comprises cyclosporin A, Triton X-705 (70%)®, and clove oil.

Another embodiment comprises cyclosporin A, Volpo S-10®, and clove oil.

Another embodiment comprises cyclosporin A, Volpo S-2®, and clove oil.

Another embodiment comprises cyclosporin A, Volpo S-20®, and clove oil.

Another embodiment comprises cyclosporin A, Volpo-10®, and clove oil.

Another embodiment comprises cyclosporin A, Volpo-20®, and clove oil.

Another embodiment comprises cyclosporin A, Volpo-3®, and clove oil.

Another embodiment comprises cyclosporin A, Volpo-5®, and clove oil.

Another embodiment comprises cyclosporin A, Abex 2515®, and Cassia oil.

Another embodiment comprises cyclosporin A, Abex 2525/40®, and Cassia oil.

Another embodiment comprises cyclosporin A, Abex 2535®, and Cassia oil.

Another embodiment comprises cyclosporin A, Abex 2545®, and Cassia oil.

Another embodiment comprises cyclosporin A, Acconon E®, and Cassia oil.

Another embodiment comprises cyclosporin A, Alfonic 1216CO-9®, and Cassia oil.

Another embodiment comprises cyclosporin A, Alfonic 1412-3®, and Cassia oil.

Another embodiment comprises cyclosporin A, Alfonic TDA-12®, and Cassia oil.

Another embodiment comprises cyclosporin A, Alfonic TDA-3®, and Cassia oil.

Another embodiment comprises cyclosporin A, Alfonic TDA-4®, and Cassia oil.

Another embodiment comprises cyclosporin A, Alfonic TDA-6®, and Cassia oil.

Another embodiment comprises cyclosporin A, Alfonic TDA-7®, and Cassia oil.

Another embodiment comprises cyclosporin A, Alfonic TDA-8®, and Cassia oil.

Another embodiment comprises cyclosporin A, Alfonic TDA-9®, and Cassia oil.

Another embodiment comprises cyclosporin A, Alkamuls 400-DO®, and Cassia oil.

Another embodiment comprises cyclosporin A, Alkamuls 400-MO/E®, and Cassia oil.

Another embodiment comprises cyclosporin A, Ameroxol OE-10®, and Cassia oil.

Another embodiment comprises cyclosporin A, Ameroxol OE-2®, and Cassia oil.

Another embodiment comprises cyclosporin A, Ameroxol OE-20®, and Cassia oil.

Another embodiment comprises cyclosporin A, Armix 176®, and Cassia oil.

Another embodiment comprises cyclosporin A, Armix 180-C®, and Cassia oil.

Another embodiment comprises cyclosporin A, Armix 183®, and Cassia oil.

Another embodiment comprises cyclosporin A, Brij 35®, and Cassia oil.

Another embodiment comprises cyclosporin A, Brij 35 Liquid®, and Cassia oil.

Another embodiment comprises cyclosporin A, Brij 35 SP®, and Cassia oil.

Another embodiment comprises cyclosporin A, Canasol MJ 2109®, and Cassia oil.

Another embodiment comprises cyclosporin A, Canasol MJ 52®, and Cassia oil.

Another embodiment comprises cyclosporin A, Canasol R 3600®, and Cassia oil.

Another embodiment comprises cyclosporin A, Canasol R 3603®, and Cassia oil.

Another embodiment comprises cyclosporin A, Canasol R 4000 H®, and Cassia oil.

Another embodiment comprises cyclosporin A, Cedepal CA-210®, and Cassia oil.

Another embodiment comprises cyclosporin A, Cedepal CA-520®, and Cassia oil.

Another embodiment comprises cyclosporin A, Cedepal CA-630®, and Cassia oil.

Another embodiment comprises cyclosporin A, Cedepal CA-720®, and Cassia oil.

Another embodiment comprises cyclosporin A, Cedepal CA-890®, and Cassia oil.

Another embodiment comprises cyclosporin A, Cedepal CA-897®, and Cassia oil.

Another embodiment comprises cyclosporin A, Cedepal CO-210®, and Cassia oil.

Another embodiment comprises cyclosporin A, Cedepal CO-430®, and Cassia oil.

Another embodiment comprises cyclosporin A, Cedepal CO-530®, and Cassia oil.

Another embodiment comprises cyclosporin A, Cedepal CO-630®, and Cassia oil.

Another embodiment comprises cyclosporin A, Cedepal CO-710®, and Cassia oil.

Another embodiment comprises cyclosporin A, Cedepal CO-730®, and Cassia oil.

Another embodiment comprises cyclosporin A, Cedepal CO-880®, and Cassia oil.

Another embodiment comprises cyclosporin A, Cedepal CO-887®, and Cassia oil.

Another embodiment comprises cyclosporin A, Cedepal CO-890®, and Cassia oil.

Another embodiment comprises cyclosporin A, Cedepal CO-897®, and Cassia oil.

Another embodiment comprises cyclosporin A, Cedepal CO-970®, and Cassia oil.

Another embodiment comprises cyclosporin A, Cedepal CO-977®, and Cassia oil.

Another embodiment comprises cyclosporin A, Cedepal CO-990®, and Cassia oil.

Another embodiment comprises cyclosporin A, Cedepal CO-997®, and Cassia oil.

Another embodiment comprises cyclosporin A, Chemal BP 261®, and Cassia oil.

Another embodiment comprises cyclosporin A, Chemal BP 262®, and Cassia oil.

Another embodiment comprises cyclosporin A, Chemal BP 3172®, and Cassia oil.

Another embodiment comprises cyclosporin A, Chemal BP 3174®, and Cassia oil.

Another embodiment comprises cyclosporin A, Chemal DA-4®, and Cassia oil.

Another embodiment comprises cyclosporin A, Chemal DA-6®, and Cassia oil.

Another embodiment comprises cyclosporin A, Chemal DA-9®, and Cassia oil.

Another embodiment comprises cyclosporin A, Chemal LA-23®, and Cassia oil.

Another embodiment comprises cyclosporin A, Chemal LA-4®, and Cassia oil.

Another embodiment comprises cyclosporin A, Chemal LA-9®, and Cassia oil.

Another embodiment comprises cyclosporin A, Chemal OA-20G®, and Cassia oil.

Another embodiment comprises cyclosporin A, Chemal OA-5®, and Cassia oil.

Another embodiment comprises cyclosporin A, Chemal OA-9®, and Cassia oil.

Another embodiment comprises cyclosporin A, Chemal TDA-12®, and Cassia oil.

Another embodiment comprises cyclosporin A, Chemal TDA-15®, and Cassia oil.

Another embodiment comprises cyclosporin A, Chemal TDA-3®, and Cassia oil.

Another embodiment comprises cyclosporin A, Chemal TDA-6®, and Cassia oil.

Another embodiment comprises cyclosporin A, Chemal TDA-9®, and Cassia oil.

Another embodiment comprises cyclosporin A, Chemax CO-16®, and Cassia oil.

Another embodiment comprises cyclosporin A, Chemax CO-200/50®, and Cassia oil.

Another embodiment comprises cyclosporin A, Chemax CO-25®, and Cassia oil.

Another embodiment comprises cyclosporin A, Chemax CO-30®, and Cassia oil.

Another embodiment comprises cyclosporin A, Chemax CO-36®, and Cassia oil.

Another embodiment comprises cyclosporin A, Chemax CO-40®, and Cassia oil.

Another embodiment comprises cyclosporin A, Chemax CO-5®, and Cassia oil.

Another embodiment comprises cyclosporin A, Chemax CO-80®, and Cassia oil.

Another embodiment comprises cyclosporin A, Chemax DNP-150/50®, and Cassia oil.

Another embodiment comprises cyclosporin A, Chemax DNP-18®, and Cassia oil.

Another embodiment comprises cyclosporin A, Chemax DNP-8®, and Cassia oil.

Another embodiment comprises cyclosporin A, Chemax E 200 ML®, and Cassia oil.

Another embodiment comprises cyclosporin A, Chemax E 200 MO®, and Cassia oil.

Another embodiment comprises cyclosporin A, Chemax E 400 ML®, and Cassia oil.

Another embodiment comprises cyclosporin A, Chemax E 400 MO®, and Cassia oil.

Another embodiment comprises cyclosporin A, Chemax E 600 ML®, and Cassia oil.

Another embodiment comprises cyclosporin A, Chemax E 600 MO®, and Cassia oil.

Another embodiment comprises cyclosporin A, Chemax E1000 MO®, and Cassia oil.

Another embodiment comprises cyclosporin A, Chemax E1000 MS®, and Cassia oil.

Another embodiment comprises cyclosporin A, Chemax E200 MS®, and Cassia oil.

Another embodiment comprises cyclosporin A, Chemax E400 MS®, and Cassia oil.

Another embodiment comprises cyclosporin A, Chemax E600 MS®, and Cassia oil.

Another embodiment comprises cyclosporin A, Chemax HCO-16®, and Cassia oil.

Another embodiment comprises cyclosporin A, Chemax HCO-200/50®, and Cassia oil.

Another embodiment comprises cyclosporin A, Chemax HCO-25®, and Cassia oil.

Another embodiment comprises cyclosporin A, Chemax HCO-5®, and Cassia oil.

Another embodiment comprises cyclosporin A, Chemax TO-16®, and Cassia oil.

Another embodiment comprises cyclosporin A, Chemax TO-8®, and Cassia oil.

Another embodiment comprises cyclosporin A, Colalube 3409®, and Cassia oil.

Another embodiment comprises cyclosporin A, Colalube 3411®, and Cassia oil.

Another embodiment comprises cyclosporin A, Colalube 3413®, and Cassia oil.

Another embodiment comprises cyclosporin A, Colalube 3414®, and Cassia oil.

Another embodiment comprises cyclosporin A, Colalube 3415®, and Cassia oil.

Another embodiment comprises cyclosporin A, Colalube 3416®, and Cassia oil.

Another embodiment comprises cyclosporin A, Colalube 3417®, and Cassia oil.

Another embodiment comprises cyclosporin A, Delonic NPE-100®, and Cassia oil.

Another embodiment comprises cyclosporin A, Delonic NPE-30®, and Cassia oil.

Another embodiment comprises cyclosporin A, Delonic NPE-40®, and Cassia oil.

Another embodiment comprises cyclosporin A, Delonic OPE-10®, and Cassia oil.

Another embodiment comprises cyclosporin A, Delonic OPE-12®, and Cassia oil.

Another embodiment comprises cyclosporin A, Delonic OPE-30®, and Cassia oil.

Another embodiment comprises cyclosporin A, Delonic OPE-40®, and Cassia oil.

Another embodiment comprises cyclosporin A, Delonic OPE-5®, and Cassia oil.

Another embodiment comprises cyclosporin A, Delonic OPE-7.5®, and Cassia oil.

Another embodiment comprises cyclosporin A, DePeg 16-CO®, and Cassia oil.

Another embodiment comprises cyclosporin A, DePeg 25-CO®, and Cassia oil.

Another embodiment comprises cyclosporin A, DePeg 30-CO®, and Cassia oil.

Another embodiment comprises cyclosporin A, DePeg 40-CO®, and Cassia oil.

Another embodiment comprises cyclosporin A, DePeg 5-CO®, and Cassia oil.

Another embodiment comprises cyclosporin A, DePeg 80-CO®, and Cassia oil.

Another embodiment comprises cyclosporin A, DeSonic S-100®, and Cassia oil.

Another embodiment comprises cyclosporin A, DeSonic S-114®, and Cassia oil.

Another embodiment comprises cyclosporin A, DeSonic S-405®, and Cassia oil.

Another embodiment comprises cyclosporin A, DeSonic S-45®, and Cassia oil.

Another embodiment comprises cyclosporin A, DeSonic TDA-9®, and Cassia oil.

Another embodiment comprises cyclosporin A, DeThox Acid O-14®, and Cassia oil.

Another embodiment comprises cyclosporin A, DeThox Acid O-16®, and Cassia oil.

Another embodiment comprises cyclosporin A, DeThox Acid O-9®, and Cassia oil.

Another embodiment comprises cyclosporin A, DeThox Acid TO-14®, and Cassia oil.

Another embodiment comprises cyclosporin A, DeThox Acid TO-16.5®, and Cassia oil.

Another embodiment comprises cyclosporin A, DeThox Acid TO-8.5®, and Cassia oil.

Another embodiment comprises cyclosporin A, Dur-Em 114®, and Cassia oil.

Another embodiment comprises cyclosporin A, Dur-Em 117®, and Cassia oil.

Another embodiment comprises cyclosporin A, Dur-Em 204®, and Cassia oil.

Another embodiment comprises cyclosporin A, Dur-Em 207®, and Cassia oil.

Another embodiment comprises cyclosporin A, Dur-Em 207E®, and Cassia oil.

Another embodiment comprises cyclosporin A, Emerest 2704®, and Cassia oil.

Another embodiment comprises cyclosporin A, Emerest 2712®, and Cassia oil.

Another embodiment comprises cyclosporin A, Emerest 2715®, and Cassia oil.

Another embodiment comprises cyclosporin A, Ethal EH-2®, and Cassia oil.

Another embodiment comprises cyclosporin A, Ethal EH-5®, and Cassia oil.

Another embodiment comprises cyclosporin A, Ethal OA-23®, and Cassia oil.

Another embodiment comprises cyclosporin A, Ethosperse CA-20®, and Cassia oil.

Another embodiment comprises cyclosporin A, Ethosperse LA-12®, and Cassia oil.

Another embodiment comprises cyclosporin A, Ethosperse LA-23®, and Cassia oil.

Another embodiment comprises cyclosporin A, Ethosperse LA-4®, and Cassia oil.

Another embodiment comprises cyclosporin A, Ethox 3095®, and Cassia oil.

Another embodiment comprises cyclosporin A, Ethox CO-16®, and Cassia oil.

Another embodiment comprises cyclosporin A, Ethox CO-200®, and Cassia oil.

Another embodiment comprises cyclosporin A, Ethox CO-25®, and Cassia oil.

Another embodiment comprises cyclosporin A, Ethox CO-30®, and Cassia oil.

Another embodiment comprises cyclosporin A, Ethox CO-36®, and Cassia oil.

Another embodiment comprises cyclosporin A, Ethox CO-40®, and Cassia oil.

Another embodiment comprises cyclosporin A, Ethox CO-5®, and Cassia oil.

Another embodiment comprises cyclosporin A, Ethox DO-14®, and Cassia oil.

Another embodiment comprises cyclosporin A, Ethox DO-9®, and Cassia oil.

Another embodiment comprises cyclosporin A, Ethox MA-8®, and Cassia oil.

Another embodiment comprises cyclosporin A, Ethox MI-14®, and Cassia oil.

Another embodiment comprises cyclosporin A, Ethox ML-14®, and Cassia oil.

Another embodiment comprises cyclosporin A, Ethox ML-5®, and Cassia oil.

Another embodiment comprises cyclosporin A, Ethox MO-14®, and Cassia oil.

Another embodiment comprises cyclosporin A, Ethox MO-9®, and Cassia oil.

Another embodiment comprises cyclosporin A, Ethox MS-14®, and Cassia oil.

Another embodiment comprises cyclosporin A, Ethox MS-23®, and Cassia oil.

Another embodiment comprises cyclosporin A, Ethox MS-40®, and Cassia oil.

Another embodiment comprises cyclosporin A, Ethox MS-8®, and Cassia oil.

Another embodiment comprises cyclosporin A, Ethox TO-16®, and Cassia oil.

Another embodiment comprises cyclosporin A, Ethox TO-8®, and Cassia oil.

Another embodiment comprises cyclosporin A, Ethylan SN-120®, and Cassia oil.

Another embodiment comprises cyclosporin A, Ethylan SN-70®, and Cassia oil.

Another embodiment comprises cyclosporin A, Ethylan SN-90®, and Cassia oil.

Another embodiment comprises cyclosporin A, Flexricin 13®, and Cassia oil.

Another embodiment comprises cyclosporin A, Flexricin 15®, and Cassia oil.

Another embodiment comprises cyclosporin A, Genapol 1261®, and Cassia oil.

Another embodiment comprises cyclosporin A, Generol 122 E25®, and Cassia oil.

Another embodiment comprises cyclosporin A, Glicepol 160®, and Cassia oil.

Another embodiment comprises cyclosporin A, Glicepol 560®, and Cassia oil.

Another embodiment comprises cyclosporin A, Glicepol GMS 20®, and Cassia oil.

Another embodiment comprises cyclosporin A, Hetoxide BN-13®, and Cassia oil.

Another embodiment comprises cyclosporin A, Hetoxide DNP-4®, and Cassia oil.

Another embodiment comprises cyclosporin A, Hetoxide DNP-9.6®, and Cassia oil.

Another embodiment comprises cyclosporin A, Hetoxide G-26®, and Cassia oil.

Another embodiment comprises cyclosporin A, Hetoxide G-7®, and Cassia oil.

Another embodiment comprises cyclosporin A, Hetoxide GT-20®, and Cassia oil.

Another embodiment comprises cyclosporin A, Hetoxide HC-40®, and Cassia oil.

Another embodiment comprises cyclosporin A, Hetoxol C-24®, and Cassia oil.

Another embodiment comprises cyclosporin A, Hetoxol CA-10®, and Cassia oil.

Another embodiment comprises cyclosporin A, Hetoxol CA-2®, and Cassia oil.

Another embodiment comprises cyclosporin A, Hetoxol CA-20®, and Cassia oil.

Another embodiment comprises cyclosporin A, Hetoxol CAWS®, and Cassia oil.

Another embodiment comprises cyclosporin A, Hetoxol CD-3®, and Cassia oil.

Another embodiment comprises cyclosporin A, Hetoxol CD-4®, and Cassia oil.

Another embodiment comprises cyclosporin A, Hetoxol CS-15®, and Cassia oil.

Another embodiment comprises cyclosporin A, Hetoxol CS-20®, and Cassia oil.

Another embodiment comprises cyclosporin A, Hetoxol CS-20D®, and Cassia oil.

Another embodiment comprises cyclosporin A, Hetoxol CS-25®, and Cassia oil.

Another embodiment comprises cyclosporin A, Hetoxol CS-50®, and Cassia oil.

Another embodiment comprises cyclosporin A, Hetoxol CS-9®, and Cassia oil.

Another embodiment comprises cyclosporin A, Hetoxol CSA-15®, and Cassia oil.

Another embodiment comprises cyclosporin A, Hetoxol D®, and Cassia oil.

Another embodiment comprises cyclosporin A, Hetoxol G®, and Cassia oil.

Another embodiment comprises cyclosporin A, Hetoxol J®, and Cassia oil.

Another embodiment comprises cyclosporin A, Hetoxol L®, and Cassia oil.

Another embodiment comprises cyclosporin A, Hetoxol L-23®, and Cassia oil.

Another embodiment comprises cyclosporin A, Hetoxol L-4®, and Cassia oil.

Another embodiment comprises cyclosporin A, Hetoxol L-9®, and Cassia oil.

Another embodiment comprises cyclosporin A, Hetoxol LS-9®, and Cassia oil.

Another embodiment comprises cyclosporin A, Hetoxol M-3®, and Cassia oil.

Another embodiment comprises cyclosporin A, Hetoxol OA-10 Special®, and Cassia oil.

Another embodiment comprises cyclosporin A, Hetoxol OA-20 Special®, and Cassia oil.

Another embodiment comprises cyclosporin A, Hetoxol OA-3 Special®, and Cassia oil.

Another embodiment comprises cyclosporin A, Hetoxol OA-5 Special®, and Cassia oil.

Another embodiment comprises cyclosporin A, Hetoxol OL-10®, and Cassia oil.

Another embodiment comprises cyclosporin A, Hetoxol OL-2®, and Cassia oil.

Another embodiment comprises cyclosporin A, Hetoxol OL-23®, and Cassia oil.

Another embodiment comprises cyclosporin A, Hetoxol OL-4®, and Cassia oil.

Another embodiment comprises cyclosporin A, Hetoxol OL-40®, and Cassia oil.

Another embodiment comprises cyclosporin A, Hetoxol OL-5®, and Cassia oil.

Another embodiment comprises cyclosporin A, Hetoxol PLA®, and Cassia oil.

Another embodiment comprises cyclosporin A, Hetoxol SP-15®, and Cassia oil.

Another embodiment comprises cyclosporin A, Hetoxol STA-10®, and Cassia oil.

Another embodiment comprises cyclosporin A, Hetoxol STA-2®, and Cassia oil.

Another embodiment comprises cyclosporin A, Hetoxol STA-30®, and Cassia oil.

Another embodiment comprises cyclosporin A, Hetoxol TD-12®, and Cassia oil.

Another embodiment comprises cyclosporin A, Hetoxol TD-18®, and Cassia oil.

Another embodiment comprises cyclosporin A, Hetoxol TD-3®, and Cassia oil.

Another embodiment comprises cyclosporin A, Hetoxol TD-6®, and Cassia oil.

Another embodiment comprises cyclosporin A, Hetoxol TD-9®, and Cassia oil.

Another embodiment comprises cyclosporin A, Hetoxol TDEP-15®, and Cassia oil.

Another embodiment comprises cyclosporin A, Hetoxol TDEP-63®, and Cassia oil.

Another embodiment comprises cyclosporin A, Iconol 24-12®, and Cassia oil.

Another embodiment comprises cyclosporin A, Iconol 24-23®, and Cassia oil.

Another embodiment comprises cyclosporin A, Iconol 24-4®, and Cassia oil.

Another embodiment comprises cyclosporin A, Iconol DA-4®, and Cassia oil.

Another embodiment comprises cyclosporin A, Iconol DA-6®, and Cassia oil.

Another embodiment comprises cyclosporin A, Iconol TDA-10®, and Cassia oil.

Another embodiment comprises cyclosporin A, Iconol TDA-3®, and Cassia oil.

Another embodiment comprises cyclosporin A, Iconol TDA-6®, and Cassia oil.

Another embodiment comprises cyclosporin A, Iconol TDA-8-90%®, and Cassia oil.

Another embodiment comprises cyclosporin A, Iconol TDA-9®, and Cassia oil.

Another embodiment comprises cyclosporin A, Igepal CA-210®, and Cassia oil.

Another embodiment comprises cyclosporin A, Igepal CA-407®, and Cassia oil.

Another embodiment comprises cyclosporin A, Igepal CA-420®, and Cassia oil.

Another embodiment comprises cyclosporin A, Igepal CA-520®, and Cassia oil.

Another embodiment comprises cyclosporin A, Igepal CA-620®, and Cassia oil.

Another embodiment comprises cyclosporin A, Igepal CA-630®, and Cassia oil.

Another embodiment comprises cyclosporin A, Igepal CA-720®, and Cassia oil.

Another embodiment comprises cyclosporin A, Igepal CA-877®, and Cassia oil.

Another embodiment comprises cyclosporin A, Igepal CA-887®, and Cassia oil.

Another embodiment comprises cyclosporin A, Igepal CA-897®, and Cassia oil.

Another embodiment comprises cyclosporin A, Igepal CO-210®, and Cassia oil.

Another embodiment comprises cyclosporin A, Igepal CO-430®, and Cassia oil.

Another embodiment comprises cyclosporin A, Igepal CO-520®, and Cassia oil.

Another embodiment comprises cyclosporin A, Igepal CO-530®, and Cassia oil.

Another embodiment comprises cyclosporin A, Igepal CO-610®, and Cassia oil.

Another embodiment comprises cyclosporin A, Igepal CO-630®, and Cassia oil.

Another embodiment comprises cyclosporin A, Igepal CO-630 Special®, and Cassia oil.

Another embodiment comprises cyclosporin A, Igepal CO-660®, and Cassia oil.

Another embodiment comprises cyclosporin A, Igepal CO-710®, and Cassia oil.

Another embodiment comprises cyclosporin A, Igepal CO-720®, and Cassia oil.

Another embodiment comprises cyclosporin A, Igepal CO-730®, and Cassia oil.

Another embodiment comprises cyclosporin A, Igepal CO-738®, and Cassia oil.

Another embodiment comprises cyclosporin A, Igepal CO-850®, and Cassia oil.

Another embodiment comprises cyclosporin A, Igepal CO-880®, and Cassia oil.

Another embodiment comprises cyclosporin A, Igepal CO-887®, and Cassia oil.

Another embodiment comprises cyclosporin A, Igepal CO-890®, and Cassia oil.

Another embodiment comprises cyclosporin A, Igepal CO-897®, and Cassia oil.

Another embodiment comprises cyclosporin A, Igepal CO-970®, and Cassia oil.

Another embodiment comprises cyclosporin A, Igepal CO-977®, and Cassia oil.

Another embodiment comprises cyclosporin A, Igepal CO-987®, and Cassia oil.

Another embodiment comprises cyclosporin A, Igepal CO-990 FLK®, and Cassia oil.

Another embodiment comprises cyclosporin A, Igepal CO-997®, and Cassia oil.

Another embodiment comprises cyclosporin A, Igepal DM-430®, and Cassia oil.

Another embodiment comprises cyclosporin A, Igepal DM-530®, and Cassia oil.

Another embodiment comprises cyclosporin A, Igepal DM-710®, and Cassia oil.

Another embodiment comprises cyclosporin A, Igepal DM-730®, and Cassia oil.

Another embodiment comprises cyclosporin A, Igepal DM-880®, and Cassia oil.

Another embodiment comprises cyclosporin A, Igepal DM-970 FLK®, and Cassia oil.

Another embodiment comprises cyclosporin A, Igepal OD-410®, and Cassia oil.

Another embodiment comprises cyclosporin A, Igepal RC-520®, and Cassia oil.

Another embodiment comprises cyclosporin A, Igepal RC-620®, and Cassia oil.

Another embodiment comprises cyclosporin A, Igepal RC-630®, and Cassia oil.

Another embodiment comprises cyclosporin A, Lexemul CS-20®, and Cassia oil.

Another embodiment comprises cyclosporin A, Lipo GMS 450®, and Cassia oil.

Another embodiment comprises cyclosporin A, Lipo Polyglycol 1000®, and Cassia oil.

Another embodiment comprises cyclosporin A, Lipo Polyglycol 200®, and Cassia oil.

Another embodiment comprises cyclosporin A, Lipo Polyglycol 300®, and Cassia oil.

Another embodiment comprises cyclosporin A, Lipo Polyglycol 3350®, and Cassia oil.

Another embodiment comprises cyclosporin A, Lipo Polyglycol 400®, and Cassia oil.

Another embodiment comprises cyclosporin A, Lipo Polyglycol 600®, and Cassia oil.

Another embodiment comprises cyclosporin A, Lipocol C-10®, and Cassia oil.

Another embodiment comprises cyclosporin A, Lipocol C-2®, and Cassia oil.

Another embodiment comprises cyclosporin A, Lipocol C-20®, and Cassia oil.

Another embodiment comprises cyclosporin A, Lipocol L-12®, and Cassia oil.

Another embodiment comprises cyclosporin A, Lipocol L-23®, and Cassia oil.

Another embodiment comprises cyclosporin A, Lipocol L-4®, and Cassia oil.

Another embodiment comprises cyclosporin A, Lipocol O-10®, and Cassia oil.

Another embodiment comprises cyclosporin A, Lipocol O-2®, and Cassia oil.

Another embodiment comprises cyclosporin A, Lipocol O-20®, and Cassia oil.

Another embodiment comprises cyclosporin A, Lipocol O-5®, and Cassia oil.

Another embodiment comprises cyclosporin A, Lipocol S-10®, and Cassia oil.

Another embodiment comprises cyclosporin A, Lipocol S-2®, and Cassia oil.

Another embodiment comprises cyclosporin A, Lipocol S-20®, and Cassia oil.

Another embodiment comprises cyclosporin A, Lipocol SC-15®, and Cassia oil.

Another embodiment comprises cyclosporin A, Lipocol SC-20®, and Cassia oil.

Another embodiment comprises cyclosporin A, Lipocol SC-4®, and Cassia oil.

Another embodiment comprises cyclosporin A, Lipocol TD-12®, and Cassia oil.

Another embodiment comprises cyclosporin A, Lumulse 40-O K®, and Cassia oil.

Another embodiment comprises cyclosporin A, Lumulse 42-O K®, and Cassia oil.

Another embodiment comprises cyclosporin A, Lumulse GMT-40®, and Cassia oil.

Another embodiment comprises cyclosporin A, Lumulse PEG 1450®, and Cassia oil.

Another embodiment comprises cyclosporin A, Lumulse PEG 1450 NF®, and Cassia oil.

Another embodiment comprises cyclosporin A, Lumulse PEG 8000®, and Cassia oil.

Another embodiment comprises cyclosporin A, Lumulse PGO®, and Cassia oil.

Another embodiment comprises cyclosporin A, Lumulse POE (2) Oleyl Amine®, and Cassia oil.

Another embodiment comprises cyclosporin A, Magrabar PGO-104®, and Cassia oil.

Another embodiment comprises cyclosporin A, Mapeg 200 ML®, and Cassia oil.

Another embodiment comprises cyclosporin A, Mapeg 400 MO®, and Cassia oil.

Another embodiment comprises cyclosporin A, Mapeg 400 MOT®, and Cassia oil.

Another embodiment comprises cyclosporin A, Mapeg 400DO®, and Cassia oil.

Another embodiment comprises cyclosporin A, Mapeg 400DOT®, and Cassia oil.

Another embodiment comprises cyclosporin A, Mapeg 600DOT®, and Cassia oil.

Another embodiment comprises cyclosporin A, Marlox B 24/80®, and Cassia oil.

Another embodiment comprises cyclosporin A, Marlox FK 86®, and Cassia oil.

Another embodiment comprises cyclosporin A, Marlox MO 154®, and Cassia oil.

Another embodiment comprises cyclosporin A, Mazox CDA CG®, and Cassia oil.

Another embodiment comprises cyclosporin A, Monoglycerides Glyceryl Monestearate®, and Cassia oil.

Another embodiment comprises cyclosporin A, Myrj 45®, and Cassia oil.

Another embodiment comprises cyclosporin A, Myrj 52®, and Cassia oil.

Another embodiment comprises cyclosporin A, Myrj 52S®, and Cassia oil.

Another embodiment comprises cyclosporin A, Myrj 53®, and Cassia oil.

Another embodiment comprises cyclosporin A, Myrj 59®, and Cassia oil.

Another embodiment comprises cyclosporin A, Norfox NP-12®, and Cassia oil.

Another embodiment comprises cyclosporin A, Norfox NP-4®, and Cassia oil.

Another embodiment comprises cyclosporin A, Norfox NP-6®, and Cassia oil.

Another embodiment comprises cyclosporin A, Norfox NP-887®, and Cassia oil.

Another embodiment comprises cyclosporin A, Norfox NP-9®, and Cassia oil.

Another embodiment comprises cyclosporin A, Norfox NP-977®, and Cassia oil.

Another embodiment comprises cyclosporin A, Norfox OP-100®, and Cassia oil.

Another embodiment comprises cyclosporin A, Norfox OP-102®, and Cassia oil.

Another embodiment comprises cyclosporin A, Norfox OP-114®, and Cassia oil.

Another embodiment comprises cyclosporin A, Norfox OP-45®, and Cassia oil.

Another embodiment comprises cyclosporin A, Pegosperse 100-L®, and Cassia oil.

Another embodiment comprises cyclosporin A, Pegosperse 100-O®, and Cassia oil.

Another embodiment comprises cyclosporin A, Pegosperse 100-S®, and Cassia oil.

Another embodiment comprises cyclosporin A, Pegosperse 1500-MS®, and Cassia oil.

Another embodiment comprises cyclosporin A, Pegosperse 1750-MS®, and Cassia oil.

Another embodiment comprises cyclosporin A, Pegosperse 200 DL®, and Cassia oil.

Another embodiment comprises cyclosporin A, Pegosperse 200-ML®, and Cassia oil.

Another embodiment comprises cyclosporin A, Pegosperse 400-DL®, and Cassia oil.

Another embodiment comprises cyclosporin A, Pegosperse 400-DO®, and Cassia oil.

Another embodiment comprises cyclosporin A, Pegosperse 400-DS®, and Cassia oil.

Another embodiment comprises cyclosporin A, Pegosperse 400-ML®, and Cassia oil.

Another embodiment comprises cyclosporin A, Pegosperse 400-MO®, and Cassia oil.

Another embodiment comprises cyclosporin A, Pegosperse 400-MOT®, and Cassia oil.

Another embodiment comprises cyclosporin A, Pegosperse 400-MS®, and Cassia oil.

Another embodiment comprises cyclosporin A, Pegosperse 50-DS®, and Cassia oil.

Another embodiment comprises cyclosporin A, Pegosperse 50-MS®, and Cassia oil.

Another embodiment comprises cyclosporin A, Pegosperse 600-ML®, and Cassia oil.

Another embodiment comprises cyclosporin A, Pegosperse 600-MS®, and Cassia oil.

Another embodiment comprises cyclosporin A, Plurafac A-38®, and Cassia oil.

Another embodiment comprises cyclosporin A, Plurafac B-25-5®, and Cassia oil.

Another embodiment comprises cyclosporin A, Plurafac B-26®, and Cassia oil.

Another embodiment comprises cyclosporin A, Plurafac D-25®, and Cassia oil.

Another embodiment comprises cyclosporin A, Plurafac LF 131®, and Cassia oil.

Another embodiment comprises cyclosporin A, Plurafac LF 4030®, and Cassia oil.

Another embodiment comprises cyclosporin A, Plurafac LF 7000®, and Cassia oil.

Another embodiment comprises cyclosporin A, Plurafac RA-20®, and Cassia oil.

Another embodiment comprises cyclosporin A, Plurafac RA-30®, and Cassia oil.

Another embodiment comprises cyclosporin A, Plurafac RA-40®, and Cassia oil.

Another embodiment comprises cyclosporin A, Pluronic 10R5®, and Cassia oil.

Another embodiment comprises cyclosporin A, Pluronic 17R2®, and Cassia oil.

Another embodiment comprises cyclosporin A, Pluronic 17R4®, and Cassia oil.

Another embodiment comprises cyclosporin A, Pluronic 25R2®, and Cassia oil.

Another embodiment comprises cyclosporin A, Pluronic 25R4®, and Cassia oil.

Another embodiment comprises cyclosporin A, Pluronic 31R1®, and Cassia oil.

Another embodiment comprises cyclosporin A, Pluronic F108®, and Cassia oil.

Another embodiment comprises cyclosporin A, Pluronic F127®, and Cassia oil.

Another embodiment comprises cyclosporin A, Pluronic F38®, and Cassia oil.

Another embodiment comprises cyclosporin A, Pluronic F68®, and Cassia oil.

Another embodiment comprises cyclosporin A, Pluronic F68LF®, and Cassia oil.

Another embodiment comprises cyclosporin A, Pluronic F77®, and Cassia oil.

Another embodiment comprises cyclosporin A, Pluronic F87®, and Cassia oil.

Another embodiment comprises cyclosporin A, Pluronic F88®, and Cassia oil.

Another embodiment comprises cyclosporin A, Pluronic F98®, and Cassia oil.

Another embodiment comprises cyclosporin A, Pluronic L10®, and Cassia oil.

Another embodiment comprises cyclosporin A, Pluronic L101®, and Cassia oil.

Another embodiment comprises cyclosporin A, Pluronic L121®, and Cassia oil.

Another embodiment comprises cyclosporin A, Pluronic L31®, and Cassia oil.

Another embodiment comprises cyclosporin A, Pluronic L35®, and Cassia oil.

Another embodiment comprises cyclosporin A, Pluronic L43®, and Cassia oil.

Another embodiment comprises cyclosporin A, Pluronic L44®, and Cassia oil.

Another embodiment comprises cyclosporin A, Pluronic L61®, and Cassia oil.

Another embodiment comprises cyclosporin A, Pluronic L62®, and Cassia oil.

Another embodiment comprises cyclosporin A, Pluronic L62D®, and Cassia oil.

Another embodiment comprises cyclosporin A, Pluronic L62LF®, and Cassia oil.

Another embodiment comprises cyclosporin A, Pluronic L64®, and Cassia oil.

Another embodiment comprises cyclosporin A, Pluronic L81®, and Cassia oil.

Another embodiment comprises cyclosporin A, Pluronic L92®, and Cassia oil.

Another embodiment comprises cyclosporin A, Pluronic P103®, and Cassia oil.

Another embodiment comprises cyclosporin A, Pluronic P104®, and Cassia oil.

Another embodiment comprises cyclosporin A, Pluronic P105®, and Cassia oil.

Another embodiment comprises cyclosporin A, Pluronic P123®, and Cassia oil.

Another embodiment comprises cyclosporin A, Pluronic P65®, and Cassia oil.

Another embodiment comprises cyclosporin A, Pluronic P84®, and Cassia oil.

Another embodiment comprises cyclosporin A, Pluronic P85®, and Cassia oil.

Another embodiment comprises cyclosporin A, Polyaldo 10-2-P®, and Cassia oil.

Another embodiment comprises cyclosporin A, Polyaldo DGDO®, and Cassia oil.

Another embodiment comprises cyclosporin A, Polyaldo HGDS®, and Cassia oil.

Another embodiment comprises cyclosporin A, Polyaldo TGMS®, and Cassia oil.

Another embodiment comprises cyclosporin A, Protegin V®, and Cassia oil.

Another embodiment comprises cyclosporin A, Protegin W®, and Cassia oil.

Another embodiment comprises cyclosporin A, Protegin WX®, and Cassia oil.

Another embodiment comprises cyclosporin A, Protegin XV®, and Cassia oil.

Another embodiment comprises cyclosporin A, Rhodasurf BC-420®, and Cassia oil.

Another embodiment comprises cyclosporin A, Rhodasurf BC-610®, and Cassia oil.

Another embodiment comprises cyclosporin A, Rhodasurf BC-630®, and Cassia oil.

Another embodiment comprises cyclosporin A, Rhodasurf BC-720®, and Cassia oil.

Another embodiment comprises cyclosporin A, Rhodasurf BC-840®, and Cassia oil.

Another embodiment comprises cyclosporin A, Rhodasurf L 1®, and Cassia oil.

Another embodiment comprises cyclosporin A, Rhodasurf L 12®, and Cassia oil.

Another embodiment comprises cyclosporin A, Rhodasurf L 2®, and Cassia oil.

Another embodiment comprises cyclosporin A, Rhodasurf L 20®, and Cassia oil.

Another embodiment comprises cyclosporin A, Rhodasurf L 3®, and Cassia oil.

Another embodiment comprises cyclosporin A, Rhodasurf L 30®, and Cassia oil.

Another embodiment comprises cyclosporin A, Rhodasurf L 4®, and Cassia oil.

Another embodiment comprises cyclosporin A, Rhodasurf L 50®, and Cassia oil.

Another embodiment comprises cyclosporin A, Rhodasurf L 7/90®, and Cassia oil.

Another embodiment comprises cyclosporin A, Rhodasurf LA-3®, and Cassia oil.

Another embodiment comprises cyclosporin A, Rhodasurf LA-7®, and Cassia oil.

Another embodiment comprises cyclosporin A, Rhodasurf LAN-23®, and Cassia oil.

Another embodiment comprises cyclosporin A, Rhodasurf ON-870®, and Cassia oil.

Another embodiment comprises cyclosporin A, Rhodasurf ON-877®, and Cassia oil.

Another embodiment comprises cyclosporin A, Rhodasurf TDA-8/5®, and Cassia oil.

Another embodiment comprises cyclosporin A, Ritapro 100®, and Cassia oil.

Another embodiment comprises cyclosporin A, Ritapro 165®, and Cassia oil.

Another embodiment comprises cyclosporin A, Ritapro 200®, and Cassia oil.

Another embodiment comprises cyclosporin A, Ritapro 300®, and Cassia oil.

Another embodiment comprises cyclosporin A, Ritoleth 10®, and Cassia oil.

Another embodiment comprises cyclosporin A, Ritoleth 2®, and Cassia oil.

Another embodiment comprises cyclosporin A, Ritoleth 20®, and Cassia oil.

Another embodiment comprises cyclosporin A, Ritoleth 5®, and Cassia oil.

Another embodiment comprises cyclosporin A, Santone 10-10-0®, and Cassia oil.

Another embodiment comprises cyclosporin A, Santone 3-1-S XTR®, and Cassia oil.

Another embodiment comprises cyclosporin A, Santone 3-1-SH®, and Cassia oil.

Another embodiment comprises cyclosporin A, Santone 8-1-O®, and Cassia oil.

Another embodiment comprises cyclosporin A, Solulan 16®, and Cassia oil.

Another embodiment comprises cyclosporin A, Solulan 75®, and Cassia oil.

Another embodiment comprises cyclosporin A, Solulan C-24®, and Cassia oil.

Another embodiment comprises cyclosporin A, Solulan L-575®, and Cassia oil.

Another embodiment comprises cyclosporin A, Standamox CAW®, and Cassia oil.

Another embodiment comprises cyclosporin A, Surfine AZI-A®, and Cassia oil.

Another embodiment comprises cyclosporin A, Surfonic CO-15®, and Cassia oil.

Another embodiment comprises cyclosporin A, Surfonic CO-25®, and Cassia oil.

Another embodiment comprises cyclosporin A, Surfonic CO-30®, and Cassia oil.

Another embodiment comprises cyclosporin A, Surfonic CO-36®, and Cassia oil.

Another embodiment comprises cyclosporin A, Surfonic CO-42®, and Cassia oil.

Another embodiment comprises cyclosporin A, Surfonic E400-MO®, and Cassia oil.

Another embodiment comprises cyclosporin A, Surfonic L12-3®, and Cassia oil.

Another embodiment comprises cyclosporin A, Surfonic L12-6®, and Cassia oil.

Another embodiment comprises cyclosporin A, Surfonic L12-8®, and Cassia oil.

Another embodiment comprises cyclosporin A, Surfonic L24-12®, and Cassia oil.

Another embodiment comprises cyclosporin A, Surfonic L24-2®, and Cassia oil.

Another embodiment comprises cyclosporin A, Surfonic L24-22®, and Cassia oil.

Another embodiment comprises cyclosporin A, Surfonic L24-3®, and Cassia oil.

Another embodiment comprises cyclosporin A, Surfonic L24-4®, and Cassia oil.

Another embodiment comprises cyclosporin A, Surfonic L24-5®, and Cassia oil.

Another embodiment comprises cyclosporin A, Surfonic L24-7®, and Cassia oil.

Another embodiment comprises cyclosporin A, Surfonic L24-7.1®, and Cassia oil.

Another embodiment comprises cyclosporin A, Surfonic L24-9®, and Cassia oil.

Another embodiment comprises cyclosporin A, Surfonic LF-17®, and Cassia oil.

Another embodiment comprises cyclosporin A, Surfonic LF-18®, and Cassia oil.

Another embodiment comprises cyclosporin A, Surfonic LF-37®, and Cassia oil.

Another embodiment comprises cyclosporin A, Surfonic LF-41®, and Cassia oil.

Another embodiment comprises cyclosporin A, Surfonic LF-42®, and Cassia oil.

Another embodiment comprises cyclosporin A, Surfonic LF-47®, and Cassia oil.

Another embodiment comprises cyclosporin A, Surfonic LF-68®, and Cassia oil.

Another embodiment comprises cyclosporin A, Surfonic N-10®, and Cassia oil.

Another embodiment comprises cyclosporin A, Surfonic N-100®, and Cassia oil.

Another embodiment comprises cyclosporin A, Surfonic N-102®, and Cassia oil.

Another embodiment comprises cyclosporin A, Surfonic N-120®, and Cassia oil.

Another embodiment comprises cyclosporin A, Surfonic N-150®, and Cassia oil.

Another embodiment comprises cyclosporin A, Surfonic N-200®, and Cassia oil.

Another embodiment comprises cyclosporin A, Surfonic N-300®, and Cassia oil.

Another embodiment comprises cyclosporin A, Surfonic N-31.5®, and Cassia oil.

Another embodiment comprises cyclosporin A, Surfonic N-40®, and Cassia oil.

Another embodiment comprises cyclosporin A, Surfonic N-400®, and Cassia oil.

Another embodiment comprises cyclosporin A, Surfonic N-60®, and Cassia oil.

Another embodiment comprises cyclosporin A, Surfonic N-85®, and Cassia oil.

Another embodiment comprises cyclosporin A, Surfonic N-95®, and Cassia oil.

Another embodiment comprises cyclosporin A, Surfonic NB-18®, and Cassia oil.

Another embodiment comprises cyclosporin A, Surfonic NB-23®, and Cassia oil.

Another embodiment comprises cyclosporin A, Surfonic NB-25®, and Cassia oil.

Another embodiment comprises cyclosporin A, Surfonic NB-307®, and Cassia oil.

Another embodiment comprises cyclosporin A, Surfonic NB-31®, and Cassia oil.

Another embodiment comprises cyclosporin A, Surfonic NB-41®, and Cassia oil.

Another embodiment comprises cyclosporin A, Surfonic NB-43®, and Cassia oil.

Another embodiment comprises cyclosporin A, Surfonic OP-100®, and Cassia oil.

Another embodiment comprises cyclosporin A, Surfonic OP-120®, and Cassia oil.

Another embodiment comprises cyclosporin A, Surfonic OP-15®, and Cassia oil.

Another embodiment comprises cyclosporin A, Surfonic OP-35®, and Cassia oil.

Another embodiment comprises cyclosporin A, Surfonic OP-70®, and Cassia oil.

Another embodiment comprises cyclosporin A, Surfonic P1®, and Cassia oil.

Another embodiment comprises cyclosporin A, Surfonic P3®, and Cassia oil.

Another embodiment comprises cyclosporin A, Surfonic P5®, and Cassia oil.

Another embodiment comprises cyclosporin A, Surfonic P6®, and Cassia oil.

Another embodiment comprises cyclosporin A, Surfonic POA-17R2®, and Cassia oil.

Another embodiment comprises cyclosporin A, Surfonic POA-25R2®, and Cassia oil.

Another embodiment comprises cyclosporin A, Surfonic TDA-11®, and Cassia oil.

Another embodiment comprises cyclosporin A, Surfonic TDA-3B®, and Cassia oil.

Another embodiment comprises cyclosporin A, Surfonic TDA-6®, and Cassia oil.

Another embodiment comprises cyclosporin A, Surfonic TDA-6/88®, and Cassia oil.

Another embodiment comprises cyclosporin A, Surfonic TDA-8®, and Cassia oil.

Another embodiment comprises cyclosporin A, Surfonic TDA-8/90®, and Cassia oil.

Another embodiment comprises cyclosporin A, Surfonic TDA-9®, and Cassia oil.

Another embodiment comprises cyclosporin A, Surfonic TX-CCN®, and Cassia oil.

Another embodiment comprises cyclosporin A, Tagat CH 40®, and Cassia oil.

Another embodiment comprises cyclosporin A, Tagat CH 60®, and Cassia oil.

Another embodiment comprises cyclosporin A, Tagat L 2®, and Cassia oil.

Another embodiment comprises cyclosporin A, Tagat S®, and Cassia oil.

Another embodiment comprises cyclosporin A, Tagat S 2®, and Cassia oil.

Another embodiment comprises cyclosporin A, Tally 100 Plus®, and Cassia oil.

Another embodiment comprises cyclosporin A, T-Det A106®, and Cassia oil.

Another embodiment comprises cyclosporin A, T-Det A109®, and Cassia oil.

Another embodiment comprises cyclosporin A, T-Det A136®, and Cassia oil.

Another embodiment comprises cyclosporin A, T-Det A139®, and Cassia oil.

Another embodiment comprises cyclosporin A, T-Det A24120, and Cassia oil.

Another embodiment comprises cyclosporin A, T-Det A243®, and Cassia oil.

Another embodiment comprises cyclosporin A, T-Det A247®, and Cassia oil.

Another embodiment comprises cyclosporin A, T-Det A249®, and Cassia oil.

Another embodiment comprises cyclosporin A, T-Det A467®, and Cassia oil.

Another embodiment comprises cyclosporin A, T-Det COE®, and Cassia oil.

Another embodiment comprises cyclosporin A, T-Det DD-10®, and Cassia oil.

Another embodiment comprises cyclosporin A, T-Det DD-5®, and Cassia oil.

Another embodiment comprises cyclosporin A, T-Det DD-7®, and Cassia oil.

Another embodiment comprises cyclosporin A, T-Det EPO-104®, and Cassia oil.

Another embodiment comprises cyclosporin A, T-Det EPO-61®, and Cassia oil.

Another embodiment comprises cyclosporin A, T-Det EPO-62®, and Cassia oil.

Another embodiment comprises cyclosporin A, T-Det EPO-64®, and Cassia oil.

Another embodiment comprises cyclosporin A, T-Det N-1.5®, and Cassia oil.

Another embodiment comprises cyclosporin A, T-Det N-10.5®, and Cassia oil.

Another embodiment comprises cyclosporin A, T-Det N-100®, and Cassia oil.

Another embodiment comprises cyclosporin A, T-Det N-1007®, and Cassia oil.

Another embodiment comprises cyclosporin A, T-Det N-12®, and Cassia oil.

Another embodiment comprises cyclosporin A, T-Det N-14®, and Cassia oil.

Another embodiment comprises cyclosporin A, T-Det N-20®, and Cassia oil.

Another embodiment comprises cyclosporin A, T-Det N-30®, and Cassia oil.

Another embodiment comprises cyclosporin A, T-Det N-307®, and Cassia oil.

Another embodiment comprises cyclosporin A, T-Det N-4®, and Cassia oil.

Another embodiment comprises cyclosporin A, T-Det N-40®, and Cassia oil.

Another embodiment comprises cyclosporin A, T-Det N-407®, and Cassia oil.

Another embodiment comprises cyclosporin A, T-Det N-50®, and Cassia oil.

Another embodiment comprises cyclosporin A, T-Det N-507®, and Cassia oil.

Another embodiment comprises cyclosporin A, T-Det N-6®, and Cassia oil.

Another embodiment comprises cyclosporin A, T-Det N-70®, and Cassia oil.

Another embodiment comprises cyclosporin A, T-Det N-8®, and Cassia oil.

Another embodiment comprises cyclosporin A, T-Det N-9.5®, and Cassia oil.

Another embodiment comprises cyclosporin A, T-Det O-12®, and Cassia oil.

Another embodiment comprises cyclosporin A, T-Det O-165®, and Cassia oil.

Another embodiment comprises cyclosporin A, T-Det O-307®, and Cassia oil.

Another embodiment comprises cyclosporin A, T-Det O-40®, and Cassia oil.

Another embodiment comprises cyclosporin A, T-Det O-407®, and Cassia oil.

Another embodiment comprises cyclosporin A, T-Det O-6®, and Cassia oil.

Another embodiment comprises cyclosporin A, T-Det O-8®, and Cassia oil.

Another embodiment comprises cyclosporin A, T-Det O-9®, and Cassia oil.

Another embodiment comprises cyclosporin A, Tegin®, and Cassia oil.

Another embodiment comprises cyclosporin A, Tergitol 15-S-12®, and Cassia oil.

Another embodiment comprises cyclosporin A, Tergitol 15-S-15®, and Cassia oil.

Another embodiment comprises cyclosporin A, Tergitol 15-S-20®, and Cassia oil.

Another embodiment comprises cyclosporin A, Tergitol 15-S-20 (80AQ)®, and Cassia oil.

Another embodiment comprises cyclosporin A, Tergitol 15-S-3®, and Cassia oil.

Another embodiment comprises cyclosporin A, Tergitol 15-S-30®, and Cassia oil.

Another embodiment comprises cyclosporin A, Tergitol 15-S-40®, and Cassia oil.

Another embodiment comprises cyclosporin A, Tergitol 15-S-40 (70AQ)®, and Cassia oil.

Another embodiment comprises cyclosporin A, Tergitol 15-S-5®, and Cassia oil.

Another embodiment comprises cyclosporin A, Tergitol 15-S-7®, and Cassia oil.

Another embodiment comprises cyclosporin A, Tergitol 15-S-9®, and Cassia oil.

Another embodiment comprises cyclosporin A, Tergitol L-101®, and Cassia oil.

Another embodiment comprises cyclosporin A, Tergitol L-61®, and Cassia oil.

Another embodiment comprises cyclosporin A, Tergitol L-62®, and Cassia oil.

Another embodiment comprises cyclosporin A, Tergitol L-64®, and Cassia oil.

Another embodiment comprises cyclosporin A, Tergitol L-81®, and Cassia oil.

Another embodiment comprises cyclosporin A, Tergitol NP-10®, and Cassia oil.

Another embodiment comprises cyclosporin A, Tergitol NP-11®, and Cassia oil.

Another embodiment comprises cyclosporin A, Tergitol NP-12®, and Cassia oil.

Another embodiment comprises cyclosporin A, Tergitol NP-13®, and Cassia oil.

Another embodiment comprises cyclosporin A, Tergitol NP-15®, and Cassia oil.

Another embodiment comprises cyclosporin A, Tergitol NP-30®, and Cassia oil.

Another embodiment comprises cyclosporin A, Tergitol NP-30 (70 AQ)®, and Cassia oil.

Another embodiment comprises cyclosporin A, Tergitol NP-4®, and Cassia oil.

Another embodiment comprises cyclosporin A, Tergitol NP-40®, and Cassia oil.

Another embodiment comprises cyclosporin A, Tergitol NP-40 (70 AQ)®, and Cassia oil.

Another embodiment comprises cyclosporin A, Tergitol NP-50 (70 AQ)®, and Cassia oil.

Another embodiment comprises cyclosporin A, Tergitol NP-55 (70 AQ)®, and Cassia oil.

Another embodiment comprises cyclosporin A, Tergitol NP-6®, and Cassia oil.

Another embodiment comprises cyclosporin A, Tergitol NP-7®, and Cassia oil.

Another embodiment comprises cyclosporin A, Tergitol NP-70 (70 AQ)®, and Cassia oil.

Another embodiment comprises cyclosporin A, Tergitol NP-8®, and Cassia oil.

Another embodiment comprises cyclosporin A, Tergitol NP-9®, and Cassia oil.

Another embodiment comprises cyclosporin A, Tergitol NP-9.5®, and Cassia oil.

Another embodiment comprises cyclosporin A, Tergitol TMN-10®, and Cassia oil.

Another embodiment comprises cyclosporin A, Tergitol TMN-3®, and Cassia oil.

Another embodiment comprises cyclosporin A, Tergitol TMN-6®, and Cassia oil.

Another embodiment comprises cyclosporin A, Tergitol XD®, and Cassia oil.

Another embodiment comprises cyclosporin A, Tergitol XH®, and Cassia oil.

Another embodiment comprises cyclosporin A, Tergitol XJ®, and Cassia oil.

Another embodiment comprises cyclosporin A, Tetronic 1107®, and Cassia oil.

Another embodiment comprises cyclosporin A, Tetronic 1307®, and Cassia oil.

Another embodiment comprises cyclosporin A, Tetronic 150R1®, and Cassia oil.

Another embodiment comprises cyclosporin A, Tetronic 304®, and Cassia oil.

Another embodiment comprises cyclosporin A, Tetronic 701®, and Cassia oil.

Another embodiment comprises cyclosporin A, Tetronic 901®, and Cassia oil.

Another embodiment comprises cyclosporin A, Tetronic 904®, and Cassia oil.

Another embodiment comprises cyclosporin A, Tetronic 908®, and Cassia oil.

Another embodiment comprises cyclosporin A, Tetronic 90R4®, and Cassia oil.

Another embodiment comprises cyclosporin A, Tomadol 1-3®, and Cassia oil.

Another embodiment comprises cyclosporin A, Tomadol 1-5®, and Cassia oil.

Another embodiment comprises cyclosporin A, Tomadol 1-7®, and Cassia oil.

Another embodiment comprises cyclosporin A, Tomadol 23-1®, and Cassia oil.

Another embodiment comprises cyclosporin A, Tomadol 23-3®, and Cassia oil.

Another embodiment comprises cyclosporin A, Tomadol 23-5®, and Cassia oil.

Another embodiment comprises cyclosporin A, Tomadol 23-6.5®, and Cassia oil.

Another embodiment comprises cyclosporin A, Tomadol 25-12®, and Cassia oil.

Another embodiment comprises cyclosporin A, Tomadol 25-3®, and Cassia oil.

Another embodiment comprises cyclosporin A, Tomadol 25-7®, and Cassia oil.

Another embodiment comprises cyclosporin A, Tomadol 25-9®, and Cassia oil.

Another embodiment comprises cyclosporin A, Tomadol 45-13®, and Cassia oil.

Another embodiment comprises cyclosporin A, Tomadol 45-2.5®, and Cassia oil.

Another embodiment comprises cyclosporin A, Tomadol 45-7®, and Cassia oil.

Another embodiment comprises cyclosporin A, Tomadol 91-2.5®, and Cassia oil.

Another embodiment comprises cyclosporin A, Tomadol 91-6®, and Cassia oil.

Another embodiment comprises cyclosporin A, Tomadol 91-8®, and Cassia oil.

Another embodiment comprises cyclosporin A, Triton X-100®, and Cassia oil.

Another embodiment comprises cyclosporin A, Triton X-102®, and Cassia oil.

Another embodiment comprises cyclosporin A, Triton X-114®, and Cassia oil.

Another embodiment comprises cyclosporin A, Triton X-15®, and Cassia oil.

Another embodiment comprises cyclosporin A, Triton X-165 (70%)®, and Cassia oil.

Another embodiment comprises cyclosporin A, Triton X-207®, and Cassia oil.

Another embodiment comprises cyclosporin A, Triton X-305 (70%)®, and Cassia oil.

Another embodiment comprises cyclosporin A, Triton X-35®, and Cassia oil.

Another embodiment comprises cyclosporin A, Triton X-405 (70%)®,and Cassia oil.

Another embodiment comprises cyclosporin A, Triton X-45®, and Cassia oil.

Another embodiment comprises cyclosporin A, Triton X-705 (70%)®, and Cassia oil.

Another embodiment comprises cyclosporin A, Volpo S-10®, and Cassia oil.

Another embodiment comprises cyclosporin A, Volpo S-2®, and Cassia oil.

Another embodiment comprises cyclosporin A, Volpo S-20®, and Cassia oil.

Another embodiment comprises cyclosporin A, Volpo-10®, and Cassia oil.

Another embodiment comprises cyclosporin A, Volpo-20®, and Cassia oil.

Another embodiment comprises cyclosporin A, Volpo-3®, and Cassia oil.

Another embodiment comprises cyclosporin A, Volpo-5®, and Cassia oil.

Another embodiment comprises cyclosporin A, Abex 2515®, and Cinnamon oil.

Another embodiment comprises cyclosporin A, Abex 2525/40®, and Cinnamon oil.

Another embodiment comprises cyclosporin A, Abex 2535®, and Cinnamon oil.

Another embodiment comprises cyclosporin A, Abex 2545®, and Cinnamon oil.

Another embodiment comprises cyclosporin A, Acconon E®, and Cinnamon oil.

Another embodiment comprises cyclosporin A, Alfonic 1216CO-9®, and Cinnamon oil.

Another embodiment comprises cyclosporin A, Alfonic 1412-3®, and Cinnamon oil.

Another embodiment comprises cyclosporin A, Alfonic TDA-12®, and Cinnamon oil.

Another embodiment comprises cyclosporin A, Alfonic TDA-3®, and Cinnamon oil.

Another embodiment comprises cyclosporin A, Alfonic TDA-4®, and Cinnamon oil.

Another embodiment comprises cyclosporin A, Alfonic TDA-6®, and Cinnamon oil.

Another embodiment comprises cyclosporin A, Alfonic TDA-7®, and Cinnamon oil.

Another embodiment comprises cyclosporin A, Alfonic TDA-8®, and Cinnamon oil.

Another embodiment comprises cyclosporin A, Alfonic TDA-9®, and Cinnamon oil.

Another embodiment comprises cyclosporin A, Alkamuls 400-DO®, and Cinnamon oil.

Another embodiment comprises cyclosporin A, Alkamuls 400-MO/E®, and Cinnamon oil.

Another embodiment comprises cyclosporin A, Ameroxol OE-10®, and Cinnamon oil.

Another embodiment comprises cyclosporin A, Ameroxol OE-2®, and Cinnamon oil.

Another embodiment comprises cyclosporin A, Ameroxol OE-20®, and Cinnamon oil.

Another embodiment comprises cyclosporin A, Armix 176®, and Cinnamon oil.

Another embodiment comprises cyclosporin A, Armix 180-C®, and Cinnamon oil.

Another embodiment comprises cyclosporin A, Armix 183®, and Cinnamon oil.

Another embodiment comprises cyclosporin A, Brij 35®, and Cinnamon oil.

Another embodiment comprises cyclosporin A, Brij 35 Liquid®, and Cinnamon oil.

Another embodiment comprises cyclosporin A, Brij 35 SP®, and Cinnamon oil.

Another embodiment comprises cyclosporin A, Canasol MJ 2109®, and Cinnamon oil.

Another embodiment comprises cyclosporin A, Canasol MJ 52®, and Cinnamon oil.

Another embodiment comprises cyclosporin A, Canasol R 3600®, and Cinnamon oil.

Another embodiment comprises cyclosporin A, Canasol R 3603®, and Cinnamon oil.

Another embodiment comprises cyclosporin A, Canasol R 4000 H®, and Cinnamon oil.

Another embodiment comprises cyclosporin A, Cedepal CA-210®, and Cinnamon oil.

Another embodiment comprises cyclosporin A, Cedepal CA-520®, and Cinnamon oil.

Another embodimert comprises cyclosporin A, Cedepal CA-630®, and Cinnamon oil.

Another embodiment comprises cyclosporin A, Cedepal CA-720®, and Cinnamon oil.

Another embodiment comprises cyclosporin A, Cedepal CA-890®, and Cinnamon oil.

Another embodiment comprises cyclosporin A, Cedepal CA-897®, and Cinnamon oil.

Another embodiment comprises cyclosporin A, Cedepal CO-210®, and Cinnamon oil.

Another embodiment comprises cyclosporin A, Cedepal CO-430®, and Cinnamon oil.

Another embodiment comprises cyclosporin A, Cedepal CO-530®, and Cinnamon oil.

Another embodiment comprises cyclosporin A, Cedepal CO-630®, and Cinnamon oil.

Another embodiment comprises cyclosporin A, Cedepal CO-710®, and Cinnamon oil.

Another embodiment comprises cyclosporin A, Cedepal CO-730®, and Cinnamon oil.

Another embodiment comprises cyclosporin A, Cedepal CO-880®, and Cinnamon oil.

Another embodiment comprises cyclosporin A, Cedepal CO-887®, and Cinnamon oil.

Another embodiment comprises cyclosporin A, Cedepal CO-890®, and Cinnamon oil.

Another embodiment comprises cyclosporin A, Cedepal CO-897®, and Cinnamon oil.

Another embodiment comprises cyclosporin A, Cedepal CO-970®, and Cinnamon oil.

Another embodiment comprises cyclosporin A, Cedepal CO-977®, and Cinnamon oil.

Another embodiment comprises cyclosporin A, Cedepal CO-990®, and Cinnamon oil.

Another embodiment comprises cyclosporin A, Cedepal CO-997®, and Cinnamon oil.

Another embodiment comprises cyclosporin A, Chemal BP 261®, and Cinnamon oil.

Another embodiment comprises cyclosporin A, Chemal BP 262®, and Cinnamon oil.

Another embodiment comprises cyclosporin A, Chemal BP 3172®, and Cinnamon oil.

Another embodiment comprises cyclosporin A, Chemal BP 3174®, and Cinnamon oil.

Another embodiment comprises cyclosporin A, Chemal DA-4®, and Cinnamon oil.

Another embodiment comprises cyclosporin A, Chemal DA-6®, and Cinnamon oil.

Another embodiment comprises cyclosporin A, Chemal DA-9®, and Cinnamon oil.

Another embodiment comprises cyclosporin A, Chemal LA-23®, and Cinnamon oil.

Another embodiment comprises cyclosporin A, Chemal LA-4®, and Cinnamon oil.

Another embodiment comprises cyclosporin A, Chemal LA-9®, and Cinnamon oil.

Another embodiment comprises cyclosporin A, Chemal OA-20G®, and Cinnamon oil.

Another embodiment comprises cyclosporin A, Chemal OA-5®, and Cinnamon oil.

Another embodiment comprises cyclosporin A, Chemal OA-9®, and Cinnamon oil.

Another embodiment comprises cyclosporin A, Chemal TDA-12®, and Cinnamon oil.

Another embodiment comprises cyclosporin A, Chemal TDA-15®, and Cinnamon oil.

Another embodiment comprises cyclosporin A, Chemal TDA-3®, and Cinnamon oil.

Another embodiment comprises cyclosporin A, Chemal TDA-6®, and Cinnamon oil.

Another embodiment comprises cyclosporin A, Chemal TDA-9®, and Cinnamon oil.

Another embodiment comprises cyclosporin A, Chemax CO-16®, and Cinnamon oil.

Another embodiment comprises cyclosporin A, Chemax CO-200/50®, and Cinnamon oil.

Another embodiment comprises cyclosporin A, Chemax CO-25®, and Cinnamon oil.

Another embodiment comprises cyclosporin A, Chemax CO-30®, and Cinnamon oil.

Another embodiment comprises cyclosporin A, Chemax CO-36®, and Cinnamon oil.

Another embodiment comprises cyclosporin A, Chemax CO-40®, and Cinnamon oil.

Another embodiment comprises cyclosporin A, Chemax CO-5®, and Cinnamon oil.

Another embodiment comprises cyclosporin A, Chemax CO-80®, and Cinnamon oil.

Another embodiment comprises cyclosporin A, Chemax DNP-150/50®, and Cinnamon oil.

Another embodiment comprises cyclosporin A, Chemax DNP-18®, and Cinnamon oil.

Another embodiment comprises cyclosporin A, Chemax DNP-8®, and Cinnamon oil.

Another embodiment comprises cyclosporin A, Chemax E 200 ML®, and Cinnamon oil.

Another embodiment comprises cyclosporin A, Chemax E 200 MO®, and Cinnamon oil.

Another embodiment comprises cyclosporin A, Chemax E 400 ML®, and Cinnamon oil.

Another embodiment comprises cyclosporin A, Chemax E 400 MO®, and Cinnamon oil.

Another embodiment comprises cyclosporin A, Chemax E 600 ML®, and Cinnamon oil.

Another embodiment comprises cyclosporin A, Chemax E 600 MO®, and Cinnamon oil.

Another embodiment comprises cyclosporin A, Chemax E1000 MO®, and Cinnamon oil.

Another embodiment comprises cyclosporin A, Chemax E1000 MS®, and Cinnamon oil.

Another embodiment comprises cyclosporin A, Chemax E200 MS®, and Cinnamon oil.

Another embodiment comprises cyclosporin A, Chemax E400 MS®, and Cinnamon oil.

Another embodiment comprises cyclosporin A, Chemax E600 MS®, and Cinnamon oil.

Another embodiment comprises cyclosporin A, Chemax HCO-16®, and Cinnamon oil.

Another embodiment comprises cyclosporin A, Chemax HCO-200/50®, and Cinnamon oil.

Another embodiment comprises cyclosporin A, Chemax HCO-25®, and Cinnamon oil.

Another embodiment comprises cyclosporin A, Chemax HCO-5®, and Cinnamon oil.

Another embodiment comprises cyclosporin A, Chemax TO-16®, and Cinnamon oil.

Another embodiment comprises cyclosporin A, Chemax TO-8®, and Cinnamon oil.

Another embodiment comprises cyclosporin A, Colalube 3409®, and Cinnamon oil.

Another embodiment comprises cyclosporin A, Colalube 3411®, and Cinnamon oil.

Another embodiment comprises cyclosporin A, Colalube 3413®, and Cinnamon oil.

Another embodiment comprises cyclosporin A, Colalube 3414®, and Cinnamon oil.

Another embodiment comprises cyclosporin A, Colalube 3415®, and Cinnamon oil.

Another embodiment comprises cyclosporin A, Colalube 3416®, and Cinnamon oil.

Another embodiment comprises cyclosporin A, Colalube 3417®, and Cinnamon oil.

Another embodiment comprises cyclosporin A, Delonic NPE-100®, and Cinnamon oil.

Another embodiment comprises cyclosporin A, Delonic NPE-30®, and Cinnamon oil.

Another embodiment comprises cyclosporin A, Delonic NPE-40®, and Cinnamon oil.

Another embodiment comprises cyclosporin A, Delonic OPE-10®, and Cinnamon oil.

Another embodiment comprises cyclosporin A, Delonic OPE-12®, and Cinnamon oil.

Another embodiment comprises cyclosporin A, Delonic OPE-30®, and Cinnamon oil.

Another embodiment comprises cyclosporin A, Delonic OPE-40®, and Cinnamon oil.

Another embodiment comprises cyclosporin A, Delonic OPE-5®, and Cinnamon oil.

Another embodiment comprises cyclosporin A, Delonic OPE-7.5®, and Cinnamon oil.

Another embodiment comprises cyclosporin A, DePeg 16-CO®, and Cinnamon oil.

Another embodiment comprises cyclosporin A, DePeg 25-C®, and Cinnamon oil.

Another embodiment comprises cyclosporin A, DePeg 30-CO®, and Cinnamon oil.

Another embodiment comprises cyclosporin A, DePeg 40-CO®, and Cinnamon oil.

Another embodiment comprises cyclosporin A, DePeg 5-CO®, and Cinnamon oil.

Another embodiment comprises cyclosporin A, DePeg 80-CO®, and Cinnamon oil.

Another embodiment comprises cyclosporin A, DeSonic S-100®, and Cinnamon oil.

Another embodiment comprises cyclosporin A, DeSonic S-114®, and Cinnamon oil.

Another embodiment comprises cyclosporin A, DeSonic S-405®, and Cinnamon oil.

Another embodiment comprises cyclosporin A, DeSonic S-45®, and Cinnamon oil.

Another embodiment comprises cyclosporin A, DeSonic TDA-9®, and Cinnamon oil.

Another embodiment comprises cyclosporin A, DeThox Acid O-14®, and Cinnamon oil.

Another embodiment comprises cyclosporin A, DeThox Acid O-16®, and Cinnamon oil.

Another embodiment comprises cyclosporin A, DeThox Acid O-9®, and Cinnamon oil.

Another embodiment comprises cyclosporin A, DeThox Acid TO-14®, and Cinnamon oil.

Another embodiment comprises cyclosporin A, DeThox Acid TO-16.5®, and Cinnamon oil.

Another embodiment comprises cyclosporin A, DeThox Acid TO-8.5®, and Cinnamon oil.

Another embodiment comprises cyclosporin A, Dur-Em 114®, and Cinnamon oil.

Another embodiment comprises cyclosporin A, Dur-Em 117®, and Cinnamon oil.

Another embodiment comprises cyclosporin A, Dur-Em 204®, and Cinnamon oil.

Another embodiment comprises cyclosporin A, Dur-Em 207®, and Cinnamon oil.

Another embodiment comprises cyclosporin A, Dur-Em 207E®, and Cinnamon oil.

Another embodiment comprises cyclosporin A, Emerest 2704®, and Cinnamon oil.

Another embodiment comprises cyclosporin A, Emerest 2712®, and Cinnamon oil.

Another embodiment comprises cyclosporin A, Emerest 2715®, and Cinnamon oil.

Another embodiment comprises cyclosporin A, Ethal EH-2®, and Cinnamon oil.

Another embodiment comprises cyclosporin A, Ethal EH-5®, and Cinnamon oil.

Another embodiment comprises cyclosporin A, Ethal OA-23®, and Cinnamon oil.

Another embodiment comprises cyclosporin A, Ethosperse CA-20®, and Cinnamon oil.

Another embodiment comprises cyclosporin A, Ethosperse LA-12®, and Cinnamon oil.

Another embodiment comprises cyclosporin A, Ethosperse LA-23®, and Cinnamon oil.

Another embodiment comprises cyclosporin A, Ethosperse LA-4®, and Cinnamon oil.

Another embodiment comprises cyclosporin A, Ethox 3095®, and Cinnamon oil.

Another embodiment comprises cyclosporin A, Ethox CO-16®, and Cinnamon oil.

Another embodiment comprises cyclosporin A, Ethox CO-200®, and Cinnamon oil.

Another embodiment comprises cyclosporin A, Ethox CO-25®, and Cinnamon oil.

Another embodiment comprises cyclosporin A, Ethox CO-30®, and Cinnamon oil.

Another embodiment comprises cyclosporin A, Ethox CO-36®, and Cinnamon oil.

Another embodiment comprises cyclosporin A, Ethox CO-40®, and Cinnamon oil.

Another embodiment comprises cyclosporin A, Ethox CO-5®, and Cinnamon oil.

Another embodiment comprises cyclosporin A, Ethox DO-14®, and Cinnamon oil.

Another embodiment comprises cyclosporin A, Ethox DO-9®, and Cinnamon oil.

Another embodiment comprises cyclosporin A, Ethox MA-8®, and Cinnamon oil.

Another embodiment comprises cyclosporin A, Ethox Ml-14®, and Cinnamon oil.

Another embodiment comprises cyclosporin A, Ethox ML-14®, and Cinnamon oil.

Another embodiment comprises cyclosporin A, Ethox ML-5®, and Cinnamon oil.

Another embodiment comprises cyclosporin A, Ethox MO-14®, and Cinnamon oil.

Another embodiment comprises cyclosporin A, Ethox MO-9®, and Cinnamon oil.

Another embodiment comprises cyclosporin A, Ethox MS-14®, and Cinnamon oil.

Another embodiment comprises cyclosporin A, Ethox MS-23®, and Cinnamon oil.

Another embodiment comprises cyclosporin A, Ethox MS-40®, and Cinnamon oil.

Another embodiment comprises cyclosporin A, Ethox MS-8®, and Cinnamon oil.

Another embodiment comprises cyclosporin A, Ethox TO-16®, and Cinnamon oil.

Another embodiment comprises cyclosporin A, Ethox TO-8®, and Cinnamon oil.

Another embodiment comprises cyclosporin A, Ethylan SN-120®, and Cinnamon oil.

Another embodiment comprises cyclosporin A, Ethylan SN-70®, and Cinnamon oil.

Another embodiment comprises cyclosporin A, Ethylan SN-90®, and Cinnamon oil.

Another embodiment comprises cyclosporin A, Flexricin 13®, and Cinnamon oil.

Another embodiment comprises cyclosporin A, Flexricin 15®, and Cinnamon oil.

Another embodiment comprises cyclosporin A, Genapol 1261®, and Cinnamon oil.

Another embodiment comprises cyclosporin A, Generol 122 E25®, and Cinnamon oil.

Another embodiment comprises cyclosporin A, Glicepol 160®, and Cinnamon oil.

Another embodiment comprises cyclosporin A, Glicepol 560®, and Cinnamon oil.

Another embodiment comprises cyclosporin A, Glicepol GMS 20®, and Cinnamon oil.

Another embodiment comprises cyclosporin A, Hetoxide BN-13®, and Cinnamon oil.

Another embodiment comprises cyclosporin A, Hetoxide DNP-4®, and Cinnamon oil.

Another embodiment comprises cyclosporin A, Hetoxide DNP-9.6®, and Cinnamon oil.

Another embodiment comprises cyclosporin A, Hetoxide G-26®, and Cinnamon oil.

Another embodiment comprises cyclosporin A, Hetoxide G-7®, and Cinnamon oil.

Another embodiment comprises cyclosporin A, Hetoxide GT-20®, and Cinnamon oil.

Another embodiment comprises cyclosporin A, Hetoxide HC-40®, and Cinnamon oil.

Another embodiment comprises cyclosporin A, Hetoxol C-24®, and Cinnamon oil.

Another embodiment comprises cyclosporin A, Hetoxol CA-10®, and Cinnamon oil.

Another embodiment comprises cyclosporin A, Hetoxol CA-2®, and Cinnamon oil.

Another embodiment comprises cyclosporin A, Hetoxol CA-20®, and Cinnamon oil.

Another embodiment comprises cyclosporin A, Hetoxol CAWS®, and Cinnamon oil.

Another embodiment comprises cyclosporin A, Hetoxol CD-3®, and Cinnamon oil.

Another embodiment comprises cyclosporin A, Hetoxol CD-4®, and Cinnamon oil.

Another embodiment comprises cyclosporin A, Hetoxol CS-15®, and Cinnamon oil.

Another embodiment comprises cyclosporin A, Hetoxol CS-20®, and Cinnamon oil.

Another embodiment comprises cyclosporin A, Hetoxol CS-20D®, and Cinnamon oil.

Another embodiment comprises cyclosporin A, Hetoxol CS-25®, and Cinnamon oil.

Another embodiment comprises cyclosporin A, Hetoxol CS-50®, and Cinnamon oil.

Another embodiment comprises cyclosporin A, Hetoxol CS-9®, and Cinnamon oil.

Another embodiment comprises cyclosporin A, Hetoxol CSA-15®, and Cinnamon oil.

Another embodiment comprises cyclosporin A, Hetoxol D®, and Cinnamon oil.

Another embodiment comprises cyclosporin A, Hetoxol G®, and Cinnamon oil.

Another embodiment comprises cyclosporin A, Hetoxol J®, and Cinnamon oil.

Another embodiment comprises cyclosporin A, Hetoxol L®, and Cinnamon oil.

Another embodiment comprises cyclosporin A, Hetoxol L-23®, and Cinnamon oil.

Another embodiment comprises cyclosporin A, Hetoxol L-4®, and Cinnamon oil.

Another embodiment comprises cyclosporin A, Hetoxol L-9®, and Cinnamon oil.

Another embodiment comprises cyclosporin A, Hetoxol LS-9®, and Cinnamon oil.

Another embodiment comprises cyclosporin A, Hetoxol M-3®, and Cinnamon oil.

Another embodiment comprises cyclosporin A, Hetoxol OA-10 Special®, and Cinnamon oil.

Another embodiment comprises cyclosporin A, Hetoxol OA-20 Special®, and Cinnamon oil.

Another embodiment comprises cyclosporin A, Hetoxol OA-3 Special®, and Cinnamon oil.

Another embodiment comprises cyclosporin A, Hetoxol OA-5 Special®, and Cinnamon oil.

Another embodiment comprises cyclosporin A, Hetoxol OL-10®, and Cinnamon oil.

Another embodiment comprises cyclosporin A, Hetoxol OL-2®, and Cinnamon oil.

Another embodiment comprises cyclosporin A, Hetoxol OL-23®, and Cinnamon oil.

Another embodiment comprises cyclosporin A, Hetoxol OL-4®, and Cinnamon oil.

Another embodiment comprises cyclosporin A, Hetoxol OL-40®, and Cinnamon oil.

Another embodiment comprises cyclosporin A, Hetoxol OL-5®, and Cinnamon oil.

Another embodiment comprises cyclosporin A, Hetoxol PLA®, and Cinnamon oil.

Another embodiment comprises cyclosporin A, Hetoxol SP-15®, and Cinnamon oil.

Another embodiment comprises cyclosporin A, Hetoxol STA-10®, and Cinnamon oil.

Another embodiment comprises cyclosporin A, Hetoxol STA-2®, and Cinnamon oil.

Another embodiment comprises cyclosporin A, Hetoxol STA-30®, and Cinnamon oil.

Another embodiment comprises cyclosporin A, Hetoxol TD-12®, and Cinnamon oil.

Another embodiment comprises cyclosporin A, Hetoxol TD-18®, and Cinnamon oil.

Another embodiment comprises cyclosporin A, Hetoxol TD-3®, and Cinnamon oil.

Another embodiment comprises cyclosporin A, Hetoxol TD-6®, and Cinnamon oil.

Another embodiment comprises cyclosporin A, Hetoxol TD-9®, and Cinnamon oil.

Another embodiment comprises cyclosporin A, Hetoxol TDEP-15®, and Cinnamon oil.

Another embodiment comprises cyclosporin A, Hetoxol TDEP-63®, and Cinnamon oil.

Another embodiment comprises cyclosporin A, Iconol 24-12®, and Cinnamon oil.

Another embodiment comprises cyclosporin A, Iconol 24-23®, and Cinnamon oil.

Another embodiment comprises cyclosporin A, Iconol 24-4®, and Cinnamon oil.

Another embodiment comprises cyclosporin A, Iconol DA-4®, and Cinnamon oil.

Another embodiment comprises cyclosporin A, Iconol DA-6®, and Cinnamon oil.

Another embodiment comprises cyclosporin A, Iconol TDA-10®, and Cinnamon oil.

Another embodiment comprises cyclosporin A, Iconol TDA-3®, and Cinnamon oil.

Another embodiment comprises cyclosporin A, Iconol TDA-6®, and Cinnamon oil.

Another embodiment comprises cyclosporin A, Iconol TDA-8-90%®, and Cinnamon oil.

Another embodiment comprises cyclosporin A, Iconol TDA-9®, and Cinnamon oil.

Another embodiment comprises cyclosporin A, Igepal CA-210®, and Cinnamon oil.

Another embodiment comprises cyclosporin A, Igepal CA-407®, and Cinnamon oil.

Another embodiment comprises cyclosporin A, Igepal CA-420®, and Cinnamon oil.

Another embodiment comprises cyclosporin A, Igepal CA-520®, and Cinnamon oil.

Another embodiment comprises cyclosporin A, Igepal CA-620®, and Cinnamon oil.

Another embodiment comprises cyclosporin A, Igepal CA-630®, and Cinnamon oil.

Another embodiment comprises cyclosporin A, Igepal CA-720®, and Cinnamon oil.

Another embodiment comprises cyclosporin A, Igepal CA-877®, and Cinnamon oil.

Another embodiment comprises cyclosporin A, Igepal CA-887®, and Cinnamon oil.

Another embodiment comprises cyclosporin A, Igepal CA-897®, and Cinnamon oil.

Another embodiment comprises cyclosporin A, Igepal CO-210®, and Cinnamon oil.

Another embodiment comprises cyclosporin A, Igepal CO-430®, and Cinnamon oil.

Another embodiment comprises cyclosporin A, Igepal CO-520®, and Cinnamon oil.

Another embodiment comprises cyclosporin A, Igepal CO-530®, and Cinnamon oil.

Another embodiment comprises cyclosporin A, Igepal CO-610®, and Cinnamon oil.

Another embodiment comprises cyclosporin A, Igepal CO-630®, and Cinnamon oil.

Another embodiment comprises cyclosporin A, Igepal CO-630 Special®, and Cinnamon oil.

Another embodiment comprises cyclosporin A, Igepal CO-660®, and Cinnamon oil.

Another embodiment comprises cyclosporin A, Igepal CO-710®, and Cinnamon oil.

Another embodiment comprises cyclosporin A, Igepal CO-720®, and Cinnamon oil.

Another embodiment comprises cyclosporin A, Igepal CO-730®, and Cinnamon oil.

Another embodiment comprises cyclosporin A, Igepal CO-738®, and Cinnamon oil.

Another embodiment comprises cyclosporin A, Igepal CO-850®, and Cinnamon oil.

Another embodiment comprises cyclosporin A, Igepal CO-880®, and Cinnamon oil.

Another embodiment comprises cyclosporin A, Igepal CO-887®, and Cinnamon oil.

Another embodiment comprises cyclosporin A, Igepal CO-890®, and Cinnamon oil.

Another embodiment comprises cyclosporin A, Igepal CO-897®, and Cinnamon oil.

Another embodiment comprises cyclosporin A, Igepal CO-970®, and Cinnamon oil.

Another embodiment comprises cyclosporin A, Igepal CO-977®, and Cinnamon oil.

Another embodiment comprises cyclosporin A, Igepal CO-987®, and Cinnamon oil.

Another embodiment comprises cyclosporin A, Igepal CO-990 FLK®, and Cinnamon oil.

Another embodiment comprises cyclosporin A, Igepal CO-997®, and Cinnamon oil.

Another embodiment comprises cyclosporin A, Igepal DM-430®, and Cinnamon oil.

Another embodiment comprises cyclosporin A, Igepal DM-530®, and Cinnamon oil.

Another embodiment comprises cyclosporin A, Igepal DM-710®, and Cinnamon oil.

Another embodiment comprises cyclosporin A, Igepal DM-730®, and Cinnamon oil.

Another embodiment comprises cyclosporin A, Igepal DM-880®, and Cinnamon oil.

Another embodiment comprises cyclosporin A, Igepal DM-970 FLK®, and Cinnamon oil.

Another embodiment comprises cyclosporin A, Igepal OD-410®, and Cinnamon oil.

Another embodiment comprises cyclosporin A, Igepal RC-520®, and Cinnamon oil.

Another embodiment comprises cyclosporin A, Igepal RC-620®, and Cinnamon oil.

Another embodiment comprises cyclosporin A, Igepal RC-630, and Cinnamon oil.

Another embodiment comprises cyclosporin A, Lexemul CS-20®, and Cinnamon oil.

Another embodiment comprises cyclosporin A, Lipo GMS 450®, and Cinnamon oil.

Another embodiment comprises cyclosporin A, Lipo Polyglycol 1000®, and Cinnamon oil.

Another embodiment comprises cyclosporin A, Lipo Polyglycol 200®, and Cinnamon oil.

Another embodiment comprises cyclosporin A, Lipo Polyglycol 300®, and Cinnamon oil.

Another embodiment comprises cyclosporin A, Lipo Polyglycol 3350®, and Cinnamon oil.

Another embodiment comprises cyclosporin A, Lipo Polyglycol 400®, and Cinnamon oil.

Another embodiment comprises cyclosporin A, Lipo Polyglycol 600®, and Cinnamon oil.

Another embodiment comprises cyclosporin A, Lipocol C-10®, and Cinnamon oil.

Another embodiment comprises cyclosporin A, Lipocol C-2®, and Cinnamon oil.

Another embodiment comprises cyclosporin A, Lipocol C-20®, and Cinnamon oil.

Another embodiment comprises cyclosporin A, Lipocol L-12®, and Cinnamon oil.

Another embodiment comprises cyclosporin A, Lipocol L-23®, and Cinnamon oil.

Another embodiment comprises cyclosporin A, Lipocol L-4®, and Cinnamon oil.

Another embodiment comprises cyclosporin A, Lipocol O-10®, and Cinnamon oil.

Another embodiment comprises cyclosporin A, Lipocol O-2®, and Cinnamon oil.

Another embodiment comprises cyclosporin A, Lipocol O-20®, and Cinnamon oil.

Another embodiment comprises cyclosporin A, Lipocol O-5®, and Cinnamon oil.

Another embodiment comprises cyclosporin A, Lipocol S-10®, and Cinnamon oil.

Another embodiment comprises cyclosporin A, Lipocol S-2®, and Cinnamon oil.

Another embodiment comprises cyclosporin A, Lipocol S-20®, and Cinnamon oil.

Another embodiment comprises cyclosporin A, Lipocol SC-15®, and Cinnamon oil.

Another embodiment comprises cyclosporin A, Lipocol SC-20®, and Cinnamon oil.

Another embodiment comprises cyclosporin A, Lipocol SC-4®, and Cinnamon oil.

Another embodiment comprises cyclosporin A, Lipocol TD-12®, and Cinnamon oil.

Another embodiment comprises cyclosporin A, Lumulse 40-O K®, and Cinnamon oil.

Another embodiment comprises cyclosporin A, Lumulse 42-O K®, and Cinnamon oil.

Another embodiment comprises cyclosporin A, Lumulse GMT-40®, and Cinnamon oil.

Another embodiment comprises cyclosporin A, Lumulse PEG 1450®, and Cinnamon oil.

Another embodiment comprises cyclosporin A, Lumulse PEG 1450 NF®, and Cinnamon oil.

Another embodiment comprises cyclosporin A, Lumulse PEG 8000®, and Cinnamon oil.

Another embodiment comprises cyclosporin A, Lumulse PGO®, and Cinnamon oil.

Another embodiment comprises cyclosporin A, Lumulse POE (2) Oleyl Amine®, and Cinnamon oil.

Another embodiment comprises cyclosporin A, Magrabar PGO-104®, and Cinnamon oil.

Another embodiment comprises cyclosporin A, Mapeg 200 ML®, and Cinnamon oil.

Another embodiment comprises cyclosporin A, Mapeg 400 MO®, and Cinnamon oil.

Another embodiment comprises cyclosporin A, Mapeg 400 MOT®, and Cinnamon oil.

Another embodiment comprises cyclosporin A, Mapeg 400DO®, and Cinnamon oil.

Another embodiment comprises cyclosporin A, Mapeg 400DOT®, and Cinnamon oil.

Another embodiment comprises cyclosporin A, Mapeg 600DOT®, and Cinnamon oil.

Another embodiment comprises cyclosporin A, Marlox B 24/80®, and Cinnamon oil.

Another embodiment comprises cyclosporin A, Marlox FK 86®, and Cinnamon oil.

Another embodiment comprises cyclosporin A, Marlox MO 154®, and Cinnamon oil.

Another embodiment comprises cyclosporin A, Mazox CDA CG®, and Cinnamon oil.

Another embodiment comprises cyclosporin A, Monoglycerides Glyceryl Monestearate®, and Cinnamon oil.

Another embodiment comprises cyclosporin A, Myrj 45®, and Cinnamon oil.

Another embodiment comprises cyclosporin A, Myrj 52®, and Cinnamon oil.

Another embodiment comprises cyclosporin A, Myrj 52S®, and Cinnamon oil.

Another embodiment comprises cyclosporin A, Myrj 53®, and Cinnamon oil.

Another embodiment comprises cyclosporin A, Myrj 59®, and Cinnamon oil.

Another embodiment comprises cyclosporin A, Norfox NP-12®, and Cinnamon oil.

Another embodiment comprises cyclosporin A, Norfox NP-4®, and Cinnamon oil.

Another embodiment comprises cyclosporin A, Norfox NP-6®, and Cinnamon oil.

Another embodiment comprises cyclosporin A, Norfox NP-887®, and Cinnamon oil.

Another embodiment comprises cyclosporin A, Norfox NP-9®, and Cinnamon oil.

Another embodiment comprises cyclosporin A, Norfox NP-977®, and Cinnamon oil.

Another embodiment comprises cyclosporin A, Norfox OP-100®, and Cinnamon oil.

Another embodiment comprises cyclosporin A, Norfox OP-102®, and Cinnamon oil.

Another embodiment comprises cyclosporin A, Norfox OP-114®, and Cinnamon oil.

Another embodiment comprises cyclosporin A, Norfox OP-45, and Cinnamon oil.

Another embodiment comprises cyclosporin A, Pegosperse 100-L®, and Cinnamon oil.

Another embodiment comprises cyclosporin A, Pegosperse 100-O®, and Cinnamon oil.

Another embodiment comprises cyclosporin A, Pegosperse 100-S®, and Cinnamon oil.

Another embodiment comprises cyclosporin A, Pegosperse 1500-MS®, and Cinnamon oil.

Another embodiment comprises cyclosporin A, Pegosperse 1750-MS®, and Cinnamon oil.

Another embodiment comprises cyclosporin A, Pegosperse 200 DL®, and Cinnamon oil.

Another embodiment comprises cyclosporin A, Pegosperse 200-ML®, and Cinnamon oil.

Another embodiment comprises cyclosporin A, Pegosperse 400-DL®, and Cinnamon oil.

Another embodiment comprises cyclosporin A, Pegosperse 400-DO®, and Cinnamon oil.

Another embodiment comprises cyclosporin A, Pegosperse 400-DS®, and Cinnamon oil.

Another embodiment comprises cyclosporin A, Pegosperse 400-ML®, and Cinnamon oil.

Another embodiment comprises cyclosporin A, Pegosperse 400-MO®, and Cinnamon oil.

Another embodiment comprises cyclosporin A, Pegosperse 400-MOT®, and Cinnamon oil.

Another embodiment comprises cyclosporin A, Pegosperse 400-MS®, and Cinnamon oil.

Another embodiment comprises cyclosporin A, Pegosperse 50-DS®, and Cinnamon oil.

Another embodiment comprises cyclosporin A, Pegosperse 50-MS®, and Cinnamon oil.

Another embodiment comprises cyclosporin A, Pegosperse 600-ML®, and Cinnamon oil.

Another embodiment comprises cyclosporin A, Pegosperse 600-MS®, and Cinnamon oil.

Another embodiment comprises cyclosporin A, Plurafac A-38®, and Cinnamon oil.

Another embodiment comprises cyclosporin A, Pluratac B-25-5®, and Cinnamon oil.

Another embodiment comprises cyclosporin A, Plurafac B-26®, and Cinnamon oil.

Another embodiment comprises cyclosporin A, Plurafac D-25®, and Cinnamon oil.

Another embodiment comprises cyclosporin A, Plurafac LF 131®, and Cinnamon oil.

Another embodiment comprises cyclosporin A, Plurafac LF 4030®, and Cinnamon oil.

Another embodiment comprises cyclosporin A, Plurafac LF 7000®, and Cinnamon oil.

Another embodiment comprises cyclosporin A, Plurafac RA-20®, and Cinnamon oil.

Another embodiment comprises cyclosporin A, Plurafac RA-30®, and Cinnamon oil.

Another embodiment comprises cyclosporin A, Plurafac RA-40®, and Cinnamon oil.

Another embodiment comprises cyclosporin A, Pluronic 10R5®, and Cinnamon oil.

Another embodiment comprises cyclosporin A, Pluronic 17R2®, and Cinnamon oil.

Another embodiment comprises cyclosporin A, Pluronic 17R4®, and Cinnamon oil.

Another embodiment comprises cyclosporin A, Pluronic 25R2®, and Cinnamon oil.

Another embodiment comprises cyclosporin A, Pluronic 25R4®, and Cinnamon oil.

Another embodiment comprises cyclosporin A, Pluronic 31 R1®, and Cinnamon oil.

Another embodiment comprises cyclosporin A, Pluronic F108®, and Cinnamon oil.

Another embodiment comprises cyclosporin A, Pluronic F127®, and Cinnamon oil.

Another embodiment comprises cyclosporin A, Pluronic F38®, and Cinnamon oil.

Another embodiment comprises cyclosporin A, Pluronic F68®, and Cinnamon oil.

Another embodiment comprises cyclosporin A, Pluronic F68LF®, and Cinnamon oil.

Another embodiment comprises cyclosporin A, Pluronic F77®, and Cinnamon oil.

Another embodiment comprises cyclosporin A, Pluronic F87®, and Cinnamon oil.

Another embodiment comprises cyclosporin A, Pluronic F88®, and Cinnamon oil.

Another embodiment comprises cyclosporin A, Pluronic F98®, and Cinnamon oil.

Another embodiment comprises cyclosporin A, Pluronic L10®, and Cinnamon oil.

Another embodiment comprises cyclosporin A, Pluronic L101®, and Cinnamon oil.

Another embodiment comprises cyclosporin A, Pluronic L121®, and Cinnamon oil.

Another embodiment comprises cyclosporin A, Pluronic L31®, and Cinnamon oil.

Another embodiment comprises cyclosporin A, Pluronic L35®, and Cinnamon oil.

Another embodiment comprises cyclosporin A, Pluronic L43®, and Cinnamon oil.

Another embodiment comprises cyclosporin A, Pluronic L44®, and Cinnamon oil.

Another embodiment comprises cyclosporin A, Pluronic L61®, and Cinnamon oil.

Another embodiment comprises cyclosporin A, Pluronic L62®, and Cinnamon oil.

Another embodiment comprises cyclosporin A, Pluronic L62D®, and Cinnamon oil.

Another embodiment comprises cyclosporin A, Pluronic L62LF®, and Cinnamon oil.

Another embodiment comprises cyclosporin A, Pluronic L64®, and Cinnamon oil.

Another embodiment comprises cyclosporin A, Pluronic L81®, and Cinnamon oil.

Another embodiment comprises cyclosporin A, Pluronic L92®, and Cinnamon oil.

Another embodiment comprises cyclosporin A, Pluronic P103®, and Cinnamon oil.

Another embodiment comprises cyclosporin A, Pluronic P104®, and Cinnamon oil.

Another embodiment comprises cyclosporin A, Pluronic P105®, and Cinnamon oil.

Another embodiment comprises cyclosporin A, Pluronic P123®, and Cinnamon oil.

Another embodiment comprises cyclosporin A, Pluronic P65®, and Cinnamon oil.

Another embodiment comprises cyclosporin A, Pluronic P84®, and Cinnamon oil.

Another embodiment comprises cyclosporin A, Pluronic P85®, and Cinnamon oil.

Another embodiment comprises cyclosporin A, Polyaldo 10-2-P®, and Cinnamon oil.

Another embodiment comprises cyclosporin A, Polyaldo DGDC®, and Cinnamon oil.

Another embodiment comprises cyclosporin A, Polyaldo HGDS®, and Cinnamon oil.

Another embodiment comprises cyclosporin A, Polyaldo TGMS®, and Cinnamon oil.

Another embodiment comprises cyclosporin A, Protegin V®, and Cinnamon oil.

Another embodiment comprises cyclosporin A, Protegin W®, and Cinnamon oil.

Another embodiment comprises cyclosporin A, Protegin WX®, and Cinnamon oil.

Another embodiment comprises cyclosporin A, Protegin XV®, and Cinnamon oil.

Another embodiment comprises cyclosporin A, Rhodasurf BC-420®, and Cinnamon oil.

Another embodiment comprises cyclosporin A, Rhodasurf BC-610®, and Cinnamon oil.

Another embodiment comprises cyclosporin A, Rhodasurf BC-630®, and Cinnamon oil.

Another embodiment comprises cyclosporin A, Rhodasurf BC-720®, and Cinnamon oil.

Another embodiment comprises cyclosporin A, Rhodasurf BC-840®, and Cinnamon oil.

Another embodiment comprises cyclosporin A, Rhodasurf L 1®, and Cinnamon oil.

Another embodiment comprises cyclosporin A, Rhodasurf L 12®, and Cinnamon oil.

Another embodiment comprises cyclosporin A, Rhodasurf L 2®, and Cinnamon oil.

Another embodiment comprises cyclosporin A, Rhodasurf L 20®, and Cinnamon oil.

Another embodiment comprises cyclosporin A, Rhodasurf L 3®, and Cinnamon oil.

Another embodiment comprises cyclosporin A, Rhodasurf L 30®, and Cinnamon oil.

Another embodiment comprises cyclosporin A, Rhodasurf L 4®, and Cinnamon oil.

Another embodiment comprises cyclosporin A, Rhodasurf L 50®, and Cinnamon oil.

Another embodiment comprises cyclosporin A, Rhodasurf L 7/90®, and Cinnamon oil.

Another embodiment comprises cyclosporin A, Rhodasurf LA-3®, and Cinnamon oil.

Another embodiment comprises cyclosporin A, Rhodasurf LA-7®, and Cinnamon oil.

Another embodiment comprises cyclosporin A, Rhodasurf LAN-23®, and Cinnamon oil.

Another embodiment comprises cyclosporin A, Rhodasurf ON-870®, and Cinnamon oil.

Another embodiment comprises cyclosporin A, Rhodasurf ON-877®, and Cinnamon oil.

Another embodiment comprises cyclosporin A, Rhodasurf TDA-8/5®, and Cinnamon oil.

Another embodiment comprises cyclosporin A, Ritapro 100®, and Cinnamon oil.

Another embodiment comprises cyclosporin A, Ritapro 165®, and Cinnamon oil.

Another embodiment comprises cyclosporin A, Ritapro 200®, and Cinnamon oil.

Another embodiment comprises cyclosporin A, Ritapro 300®, and Cinnamon oil.

Another embodiment comprises cyclosporin A, Ritoleth 10®, and Cinnamon oil.

Another embodiment comprises cyclosporin A, Ritoleth 2®, and Cinnamon oil.

Another embodiment comprises cyclosporin A, Ritoleth 20®, and Cinnamon oil.

Another embodiment comprises cyclosporin A, Ritoleth 5®, and Cinnamon oil.

Another embodiment comprises cyclosporin A, Santone 10-10-0®, and Cinnamon oil.

Another embodiment comprises cyclosporin A, Santone 3-1-S XTR®, and Cinnamon oil.

Another embodiment comprises cyclosporin A, Santone 3-1-SH®, and Cinnamon oil.

Another embodiment comprises cyclosporin A, Santone 8-1-O®, and Cinnamon oil.

Another embodiment comprises cyclosporin A, Solulan 16®, and Cinnamon oil.

Another embodiment comprises cyclosporin A, Solulan 75®, and Cinnamon oil.

Another embodiment comprises cyclosporin A, Solulan C-24®, and Cinnamon oil.

Another embodiment comprises cyclosporin A, Solulan L-575®, and Cinnamon oil.

Another embodiment comprises cyclosporin A, Standamox CAW®, and Cinnamon oil.

Another embodiment comprises cyclosporin A, Surfine AZI-A®, and Cinnamon oil.

Another embodiment comprises cyclosporin A, Surfonic CO-15®, and Cinnamon oil.

Another embodiment comprises cyclosporin A, Surfonic CO-25®, and Cinnamon oil.

Another embodiment comprises cyclosporin A, Surlonic CO-30®, and Cinnamon oil.

Another embodiment comprises cyclosporin A, Surfonic CO-36®, and Cinnamon oil.

Another embodiment comprises cyclosporin A, Surfonic CO-42®, and Cinnamon oil.

Another embodiment comprises cyclosporin A, Surfonic E400-MO®, and Cinnamon oil.

Another embodiment comprises cyclosporin A, Surfonic L12-3®, and Cinnamon oil.

Another embodiment comprises cyclosporin A, Surfonic L12-6®, and Cinnamon oil.

Another embodiment comprises cyclosporin A, Surfonic L12-8®, and Cinnamon oil.

Another embodiment comprises cyclosporin A, Surfonic L24-12®, and Cinnamon oil.

Another embodiment comprises cyclosporin A, Surfonic L24-2®, and Cinnamon oil.

Another embodiment comprises cyclosporin A, Surfonic L24-22®, and Cinnamon oil.

Another embodiment comprises cyclosporin A, Surfonic L24-3®, and Cinnamon oil.

Another embodiment comprises cyclosporin A, Surfonic L24-4®, and Cinnamon oil.

Another embodiment comprises cyclosporin A, Surfonic L24-5®, and Cinnamon oil.

Another embodiment comprises cyclosporin A, Surfonic L24-7®, and Cinnamon oil.

Another embodiment comprises cyclosporin A, Surfonic L24-7.1®, and Cinnamon oil.

Another embodiment comprises cyclosporin A, Surfonic L24-9®, and Cinnamon oil.

Another embodiment comprises cyclosporin A, Surfonic LF-17®, and Cinnamon oil.

Another embodiment comprises cyclosporin A, Surfonic LF-18®, and Cinnamon oil.

Another embodiment comprises cyclosporin A, Surfonic LF-37®, and Cinnamon oil.

Another embodiment comprises cyclosporin A, Surfonic LF-41®, and Cinnamon oil.

Another embodiment comprises cyclosporin A, Surfonic LF-42®, and Cinnamon oil.

Another embodiment comprises cyclosporin A, Surfonic LF-47®, and Cinnamon oil.

Another embodiment comprises cyclosporin A, Surfonic LF-68®, and Cinnamon oil.

Another embodiment comprises cyclosporin A, Surfonic N-10®, and Cinnamon oil.

Another embodiment comprises cyclosporin A, Surfonic N-100®, and Cinnamon oil.

Another embodiment comprises cyclosporin A, Surfonic N-102®, and Cinnamon oil.

Another embodiment comprises cyclosporin A, Surfonic N-120®, and Cinnamon oil.

Another embodiment comprises cyclosporin A, Surfonic N-150®, and Cinnamon oil.

Another embodiment comprises cyclosporin A, Surfonic N-200®, and Cinnamon oil.

Another embodiment comprises cyclosporin A, Surfonic N-300®, and Cinnamon oil.

Another embodiment comprises cyclosporin A, Surfonic N-31.5®, and Cinnamon oil.

Another embodiment comprises cyclosporin A, Surfonic N-40®, and Cinnamon oil.

Another embodiment comprises cyclosporin A, Surfonic N-400®, and Cinnamon oil.

Another embodiment comprises cyclosporin A, Surfonic N-60®, and Cinnamon oil.

Another embodiment comprises cyclosporin A, Surfonic N-85®, and Cinnamon oil.

Another embodiment comprises cyclosporin A, Surfonic N-95®, and Cinnamon oil.

Another embodiment comprises cyclosporin A, Surfonic NB-18®, and Cinnamon oil.

Another embodiment comprises cyclosporin A, Surfonic NB-23®, and Cinnamon oil.

Another embodiment comprises cyclosporin A, Surdonic NB-25®, and Cinnamon oil.

Another embodiment comprises cyclosporin A, Surfonic NB-307®, and Cinnamon oil.

Another embodiment comprises cyclosporin A, Surfonic NB-31®, and Cinnamon oil.

Another embodiment comprises cyclosporin A, Surfonic NB-41®, and Cinnamon oil.

Another embodiment comprises cyclosporin A, Surfonic NB-43®, and Cinnamon oil.

Another embodiment comprises cyclosporin A, Surfonic OP-100®, and Cinnamon oil.

Another embodiment comprises cyclosporin A, Surfonic OP-120®, and Cinnamon oil.

Another embodiment comprises cyclosporin A, Surfonic OP-15®, and Cinnamon oil.

Another embodiment comprises cyclosporin A, Surfonic OP-35®, and Cinnamon oil.

Another embodiment comprises cyclosporin A, Surfonic OP-70®, and Cinnamon oil.

Another embodiment comprises cyclosporin A, Surfonic P1®, and Cinnamon oil.

Another embodiment comprises cyclosporin A, Surfonic P3®, and Cinnamon oil.

Another embodiment comprises cyclosporin A, Surfonic P5®, and Cinnamon oil.

Another embodiment comprises cyclosporin A, Surfonic P6®, and Cinnamon oil.

Another embodiment comprises cyclosporin A, Surfonic POA-17R2®, and Cinnamon oil.

Another embodiment comprises cyclosporin A, Surfonic POA-25R2®, and Cinnamon oil.

Another embodiment comprises cyclosporin A, Surfonic TDA-11®, and Cinnamon oil.

Another embodiment comprises cyclosporin A, Surfonic TDA-3B®, and Cinnamon oil.

Another embodiment comprises cyclosporin A, Surfonic TDA-6®, and Cinnamon oil.

Another embodiment comprises cyclosporin A, Surfonic TDA-6/88®, and Cinnamon oil.

Another embodiment comprises cyclosporin A, Surfonic TDA-8®, and Cinnamon oil.

Another embodiment comprises cyclosporin A, Surfonic TDA-8/90®, and Cinnamon oil.

Another embodiment comprises cyclosporin A, Surfonic TDA-9®, and Cinnamon oil.

Another embodiment comprises cyclosporin A, Surfonic TX-CCN®, and Cinnamon oil.

Another embodiment comprises cyclosporin A, Tagat CH 40®, and Cinnamon oil.

Another embodiment comprises cyclosporin A, Tagat CH 60®, and Cinnamon oil.

Another embodiment comprises cyclosporin A, Tagat L 2®, and Cinnamon oil.

Another embodiment comprises cyclosporin A, Tagat S®, and Cinnamon oil.

Another embodiment comprises cyclosporin A, Tagat S 2®, and Cinnamon oil.

Another embodiment comprises cyclosporin A, Tally 100 Plus®, and Cinnamon oil.

Another embodiment comprises cyclosporin A, T-Det A106®, and Cinnamon oil.

Another embodiment comprises cyclosporin A, T-Det A109®, and Cinnamon oil.

Another embodiment comprises cyclosporin A, T-Det A136, and Cinnamon oil.

Another embodiment comprises cyclosporin A, T-Det A139®, and Cinnamon oil.

Another embodiment comprises cyclosporin A, T-Det A2412®, and Cinnamon oil.

Another embodiment comprises cyclosporin A, T-Det A243®, and Cinnamon oil.

Another embodiment comprises cyclosporin A, T-Det A247®, and Cinnamon oil.

Another embodiment comprises cyclosporin A, T-Det A249®, and Cinnamon oil.

Another embodiment comprises cyclosporin A, T-Det A467®, and Cinnamon oil.

Another embodiment comprises cyclosporin A, T-Det COE®, and Cinnamon oil.

Another embodiment comprises cyclosporin A, T-Det DD-10®, and Cinnamon oil.

Another embodiment comprises cyclosporin A, T-Det DD-5®, and Cinnamon oil.

Another embodiment comprises cyclosporin A, T-Det DD-7®, and Cinnamon oil.

Another embodiment comprises cyclosporin A, T-Det EPO-104®, and Cinnamon oil.

Another embodiment comprises cyclosporin A, T-Det EPO-61®, and Cinnamon oil.

Another embodiment comprises cyclosporin A, T-Det EPO-62®, and Cinnamon oil.

Another embodiment comprises cyclosporin A, T-Det EPO-64®, and Cinnamon oil.

Another embodiment comprises cyclosporin A, T-Det N-1.5®, and Cinnamon oil.

Another embodiment comprises cyclosporin A, T-Det N-10.5®, and Cinnamon oil.

Another embodiment comprises cyclosporin A, T-Det N-100®, and Cinnamon oil.

Another embodiment comprises cyclosporin A, T-Det N-1007®, and Cinnamon oil.

Another embodiment comprises cyclosporin A, T-Det N-12®, and Cinnamon oil.

Another embodiment comprises cyclosporin A, T-Det N-14®, and Cinnamon oil.

Another embodiment comprises cyclosporin A, T-Det N-20®, and Cinnamon oil.

Another embodiment comprises cyclosporin A, T-Det N-30®, and Cinnamon oil.

Another embodiment comprises cyclosporin A, T-Det N-307®, and Cinnamon oil.

Another embodiment comprises cyclosporin A, T-Det N-4®, and Cinnamon oil.

Another embodiment comprises cyclosporin A, T-Det N-40®, and Cinnamon oil.

Another embodiment comprises cyclosporin A, T-Det N-407®, and Cinnamon oil.

Another embodiment comprises cyclosporin A, T-Det N-50®, and Cinnamon oil.

Another embodiment comprises cyclosporin A, T-Det N-507®, and Cinnamon oil.

Another embodiment comprises cyclosporin A, T-Det N-6®, and Cinnamon oil.

Another embodiment comprises cyclosporin A, T-Det N-70®, and Cinnamon oil.

Another embodiment comprises cyclosporin A, T-Det N-8®, and Cinnamon oil.

Another embodiment comprises cyclosporin A, T-Det N-9.5®, and Cinnamon oil.

Another embodiment comprises cyclosporin A, T-Det O-12®, and Cinnamon oil.

Another embodiment comprises cyclosporin A, T-Det O-165®, and Cinnamon oil.

Another embodiment comprises cyclosporin A, T-Det O-307®, and Cinnamon oil.

Another embodiment comprises cyclosporin A, T-Det O-40®, and Cinnamon oil.

Another embodiment comprises cyclosporin A, T-Det O-407®, and Cinnamon oil.

Another embodiment comprises cyclosporin A, T-Det O-6®, and Cinnamon oil.

Another embodiment comprises cyclosporin A, T-Det O-8®, and Cinnamon oil.

Another embodiment comprises cyclosporin A, T-Det O-9®, and Cinnamon oil.

Another embodiment comprises cyclosporin A, Tegin®, and Cinnamon oil.

Another embodiment comprises cyclosporin A, Tergitol 15-S-12®, and Cinnamon oil.

Another embodiment comprises cyclosporin A, Tergitol 15-S-15®, and Cinnamon oil.

Another embodiment comprises cyclosporin A, Tergitol 15-S-20®, and Cinnamon oil.

Another embodiment comprises cyclosporin A, Tergitol 15-S-20 (80AQ)®, and Cinnamon oil.

Another embodiment comprises cyclosporin A, Tergitol 15-S-3®, and Cinnamon oil.

Another embodiment comprises cyclosporin A, Tergitol 15-S-30®, and Cinnamon oil.

Another embodiment comprises cyclosporin A, Tergitol 15-S-40®, and Cinnamon oil.

Another embodiment comprises cyclosporin A, Tergitol 15-S-40 (70AQ)®, and Cinnamon oil.

Another embodiment comprises cyclosporin A, Tergitol 15-S-5®, and Cinnamon oil.

Another embodiment comprises cyclosporin A, Tergitol 15-S-7®, and Cinnamon oil.

Another embodiment comprises cyclosporin A, Tergitol 15-S-9®, and Cinnamon oil.

Another embodiment comprises cyclosporin A, Tergitol L-101®, and Cinnamon oil.

Another embodiment comprises cyclosporin A, Tergitol L-61®, and Cinnamon oil.

Another embodiment comprises cyclosporin A, Tergitol L-62®, and Cinnamon oil.

Another embodiment comprises cyclosporin A, Tergitol L-64®, and Cinnamon oil.

Another embodiment comprises cyclosporin A, Tergitol L-81®, and Cinnamon oil.

Another embodiment comprises cyclosporin A, Tergitol NP-10®, and Cinnamon oil.

Another embodiment comprises cyclosporin A, Tergitol NP-11®, and Cinnamon oil.

Another embodiment comprises cyclosporin A, Tergitol NP-12®, and Cinnamon oil.

Another embodiment comprises cyclosporin A, Tergitol NP-13®, and Cinnamon oil.

Another embodiment comprises cyclosporin A, Tergitol NP-15®, and Cinnamon oil.

Another embodiment comprises cyclosporin A, Tergitol NP-30®, and Cinnamon oil.

Another embodiment comprises cyclosporin A, Tergitol NP-30 (70 AQ)®, and Cinnamon oil.

Another embodiment comprises cyclosporin A, Tergitol NP-4®, and Cinnamon oil.

Another embodiment comprises cyclosporin A, Tergitol NP-40®, and Cinnamon oil.

Another embodiment comprises cyclosporin A, Tergitol NP-40 (70 AQ)®, and Cinnamon oil.

Another embodiment comprises cyclosporin A, Tergitol NP-50 (70 AQ)®, and Cinnamon oil.

Another embodiment comprises cyclosporin A, Tergitol NP-55 (70 AQ)®, and Cinnamon oil.

Another embodiment comprises cyclosporin A, Tergitol NP-6®, and Cinnamon oil.

Another embodiment comprises cyclosporin A, Tergitol NP-7®, and Cinnamon oil.

Another embodiment comprises cyclosporin A, Tergitol NP-70 (70 AQ)®, and Cinnamon oil.

Another embodiment comprises cyclosporin A, Tergitol NP-8®, and Cinnamon oil.

Another embodiment comprises cyclosporin A, Tergitol NP-9®, and Cinnamon oil.

Another embodiment comprises cyclosporin A, Tergitol NP-9.5®, and Cinnamon oil.

Another embodiment comprises cyclosporin A, Tergitol TMN-10®, and Cinnamon oil.

Another embodiment comprises cyclosporin A, Tergitol TMN-3®, and Cinnamon oil.

Another embodiment comprises cyclosporin A, Tergitol TMN-6®, and Cinnamon oil.

Another embodiment comprises cyclosporin A, Tergitol XD®, and Cinnamon oil.

Another embodiment comprises cyclosporin A, Tergitol XH®, and Cinnamon oil.

Another embodiment comprises cyclosporin A, Tergitol XJ®, and Cinnamon oil.

Another embodiment comprises cyclosporin A, Tetronic 1107®, and Cinnamon oil.

Another embodiment comprises cyclosporin A, Tetronic 1307®, and Cinnamon oil.

Another embodiment comprises cyclosporin A, Tetronic 150R1®, and Cinnamon oil.

Another embodiment comprises cyclosporin A, Tetronic 304®, and Cinnamon oil.

Another embodiment comprises cyclosporin A, Tetronic 701®, and Cinnamon oil.

Another embodiment comprises cyclosporin A, Tetronic 901®, and Cinnamon oil.

Another embodiment comprises cyclosporin A, Tetronic 904®, and Cinnamon oil.

Another embodiment comprises cyclosporin A, Tetronic 908®, and Cinnamon oil.

Another embodiment comprises cyclosporin A, Tetronic 90R4®, and Cinnamon oil.

Another embodiment comprises cyclosporin A, Tomadol 1-3®, and Cinnamon oil.

Another embodiment comprises cyclosporin A, Tomadol 1-5®, and Cinnamon oil.

Another embodiment comprises cyclosporin A, Tomadol 1-7®, and Cinnamon oil.

Another embodiment comprises cyclosporin A, Tomadol 23-1®, and Cinnamon oil.

Another embodiment comprises cyclosporin A, Tomadol 23-3®, and Cinnamon oil.

Another embodiment comprises cyclosporin A, Tomadol 23-5®, and Cinnamon oil.

Another embodiment comprises cyclosporin A, Tomadol 23-6.5®, and Cinnamon oil.

Another embodiment comprises cyclosporin A, Tomadol 25-12®, and Cinnamon oil.

Another embodiment comprises cyclosporin A, Tomadol 25-3®, and Cinnamon oil.

Another embodiment comprises cyclosporin A, Tomadol 25-7®, and Cinnamon oil.

Another embodiment comprises cyclosporin A, Tomadol 25-9®, and Cinnamon oil.

Another embodiment comprises cyclosporin A, Tomadol 45-13®, and Cinnamon oil.

Another embodiment comprises cyclosporin A, Tomadol 45-2.5®, and Cinnamon oil.

Another embodiment comprises cyclosporin A, Tomadol 45-7®, and Cinnamon oil.

Another embodiment comprises cyclosporin A, Tomadol 91-2.5®, and Cinnamon oil.

Another embodiment comprises cyclosporin A, Tomadol 91-6®, and Cinnamon oil.

Another embodiment comprises cyclosporin A, Tomadol 91 -8®, and Cinnamon oil.

Another embodiment comprises cyclosporin A, Triton X-100®, and Cinnamon oil.

Another embodiment comprises cyclosporin A, Triton X-102®, and Cinnamon oil.

Another embodiment comprises cyclosporin A, Triton X-114®, and Cinnamon oil.

Another embodiment comprises cyclosporin A, Triton X-15®, and Cinnamon oil.

Another embodiment comprises cyclosporin A, Triton X-165 (70%)®, and Cinnamon oil.

Another embodiment comprises cyclosporin A, Triton X-207®, and Cinnamon oil.

Another embodiment comprises cyclosporin A, Triton X-305 (70%)®,and Cinnamon oil.

Another embodiment comprises cyclosporin A, Triton X-35®, and Cinnamon oil.

Another embodiment comprises cyclosporin A, Triton X-405 (70%)®, and Cinnamon oil.

Another embodiment comprises cyclosporin A, Triton X-45®, and Cinnamon oil.

Another embodiment comprises cyclosporin A, Triton X-705 (70%)®,and Cinnamon oil.

Another embodiment comprises cyclosporin A, Volpo S-10®, and Cinnamon oil.

Another embodiment comprises cyclosporin A, Volpo S-2®, and Cinnamon oil.

Another embodiment comprises cyclosporin A, Volpo S-20®, and Cinnamon oil.

Another embodiment comprises cyclosporin A, Volpo-10®, and Cinnamon oil.

Another embodiment comprises cyclosporin A, Volpo-20®, and Cinnamon oil.

Another embodiment comprises cyclosporin A, Volpo-3®, and Cinnamon oil.

Another embodiment comprises cyclosporin A, Volpo-5®, and Cinnamon oil.

Another embodiment comprises cyclosporin A, Abex 2515®, and an oil having a Specific Gravity between 0.90 and 0.95.

Another embodiment comprises cyclosporin A, Abex 2525/40®, and an oil having a Specific Gravity between 0.90 and 0.95.

Another embodiment comprises cyclosporin A, Abex 2535®, and an oil having a Specific Gravity between 0.90 and 0.95.

Another embodiment comprises cyclosporin A, Abex 2545®, and an oil having a Specific Gravity between 0.90 and 0.95.

Another embodiment comprises cyclosporin A, Acconon E®, and an oil having a Specific Gravity between 0.90 and 0.95.

Another embodiment comprises cyclosporin A, Alfonic 1216CO-9®, and an oil having a Specific Gravity between 0.90 and 0.95.

Another embodiment comprises cyclosporin A, Alfonic 1412-3®, and an oil having a Specific Gravity between 0.90 and 0.95.

Another embodiment comprises cyclosporin A, Alfonic TDA-12®, and an oil having a Specific Gravity between 0.90 and 0.95.

Another embodiment comprises cyclosporin A, Alfonic TDA-3®, and an oil having a Specific Gravity between 0.90 and 0.95.

Another embodiment comprises cyclosporin A, Alfonic TDA-4®, and an oil having a Specific Gravity between 0.90 and 0.95.

Another embodiment comprises cyclosporin A, Alfonic TDA-6®, and an oil having a Specific Gravity between 0.90 and 0.95.

Another embodiment comprises cyclosporin A, Alfonic TDA-7®, and an oil having a Specific Gravity between 0.90 and 0.95.

Another embodiment comprises cyclosporin A, Alfonic TDA-8®, and an oil having a Specific Gravity between 0.90 and 0.95.

Another embodiment comprises cyclosporin A, Alfonic TDA-9®, and an oil having a Specific Gravity between 0.90 and 0.95.

Another embodiment comprises cyclosporin A, Alkamuls 400-DO®, and an oil having a Specific Gravity between 0.90 and 0.95.

Another embodiment comprises cyclosporin A, Alkamuls 400-MO/E®, and an oil having a Specific Gravity between 0.90 and 0.95.

Another embodiment comprises cyclosporin A, Ameroxol OE-10®, and an oil having a Specific Gravity between 0.90 and 0.95.

Another embodiment comprises cyclosporin A, Ameroxol OE-2®, and an oil having a Specific Gravity between 0.90 and 0.95.

Another embodiment comprises cyclosporin A, Ameroxol OE-20®, and an oil having a Specific Gravity between 0.90 and 0.95.

Another embodiment comprises cyclosporin A, Armix 176®, and an oil having a Specific Gravity between 0.90 and 0.95.

Another embodiment comprises cyclosporin A, Armix 180-C®, and an oil having a Specific Gravity between 0.90 and 0.95.

Another embodiment comprises cyclosporin A, Armix 183®, and an oil having a Specific Gravity between 0.90 and 0.95.

Another embodiment comprises cyclosporin A, Brij 35®, and an oil having a Specific Gravity between 0.90 and 0.95.

Another embodiment comprises cyclosporin A, Brij 35 Liquid®, and an oil having a Specific Gravity between 0.90 and 0.95.

Another embodiment comprises cyclosporin A, Brij 35 SP®, and an oil having a Specific Gravity between 0.90 and 0.95.

Another embodiment comprises cyclosporin A, Canasol MJ 2109®, and an oil having a Specific Gravity between 0.90 and 0.95.

Another embodiment comprises cyclosporin A, Canasol MJ 52®, and an oil having a Specific Gravity between 0.90 and 0.95.

Another embodiment comprises cyclosporin A, Canasol R 3600®, and an oil having a Specific Gravity between 0.90 and 0.95.

Another embodiment comprises cyclosporin A, Canasol R 3603®, and an oil having a Specific Gravity between 0.90 and 0.95.

Another embodiment comprises cyclosporin A, Canasol R 4000 H®, and an oil having a Specific Gravity between 0.90 and 0.95.

Another embodiment comprises cyclosporin A, Cedepal CA-210®, and an oil having a Specific Gravity between 0.90 and 0.95.

Another embodiment comprises cyclosporin A, Cedepal CA-520®, and an oil having a Specific Gravity between 0.90 and 0.95.

Another embodiment comprises cyclosporin A, Cedepal CA-630®, and an oil having a Specific Gravity between 0.90 and 0.95.

Another embodiment comprises cyclosporin A, Cedepal CA-720®, and an oil having a Specific Gravity between 0.90 and 0.95.

Another embodiment comprises cyclosporin A, Cedepal CA-890®, and an oil having a Specific Gravity between 0.90 and 0.95.

Another embodiment comprises cyclosporin A, Cedepal CA-897®, and an oil having a Specific Gravity between 0.90 and 0.95.

Another embodiment comprises cyclosporin A, Cedepal CO-210®, and an oil having a Specific Gravity between 0.90 and 0.95.

Another embodiment comprises cyclosporin A, Cedepal CO-430®, and an oil having a Specific Gravity between 0.90 and 0.95.

Another embodiment comprises cyclosporin A, Cedepal CO-530®, and an oil having a Specific Gravity between 0.90 and 0.95.

Another embodiment comprises cyclosporin A, Cedepal CO-630®, and an oil having a Specific Gravity between 0.90 and 0.95.

Another embodiment comprises cyclosporin A, Cedepal CO-710®, and an oil having a Specific Gravity between 0.90 and 0.95.

Another embodiment comprises cyclosporin A, Cedepal CO-730®, and an oil having a Specific Gravity between 0.90 and 0.95.

Another embodiment comprises cyclosporin A, Cedepal CO-880®, and an oil having a Specific Gravity between 0.90 and 0.95.

Another embodiment comprises cyclosporin A, Cedepal CO-887®, and an oil having a Specific Gravity between 0.90 and 0.95.

Another embodiment comprises cyclosporin A, Cedepal CO-890®, and an oil having a Specific Gravity between 0.90 and 0.95.

Another embodiment comprises cyclosporin A, Cedepal CO-897®, and an oil having a Specific Gravity between 0.90 and 0.95.

Another embodiment comprises cyclosporin A, Cedepal CO-970®, and an oil having a Specific Gravity between 0.90 and 0.95.

Another embodiment comprises cyclosporin A, Cedepal CO-977®, and an oil having a Specific Gravity between 0.90 and 0.95.

Another embodiment comprises cyclosporin A, Cedepal CO-990®, and an oil having a Specific Gravity between 0.90 and 0.95.

Another embodiment comprises cyclosporin A, Cedepal CO-997®, and an oil having a Specific Gravity between 0.90 and 0.95.

Another embodiment comprises cyclosporin A, Chemal BP 261®, and an oil having a Specific Gravity between 0.90 and 0.95.

Another embodiment comprises cyclosporin A, Chemal BP 262®, and an oil having a Specific Gravity between 0.90 and 0.95.

Another embodiment comprises cyclosporin A, Chemal BP 3172®, and an oil having a Specific Gravity between 0.90 and 0.95.

Another embodiment comprises cyclosporin A, Chemal BP 3174®, and an oil having a Specific Gravity between 0.90 and 0.95.

Another embodiment comprises cyclosporin A, Chemal DA-4®, and an oil having a Specific Gravity between 0.90 and 0.95.

Another embodiment comprises cyclosporin A, Chemal DA-6®, and an oil having a Specific Gravity between 0.90 and 0.95.

Another embodiment comprises cyclosporin A, Chemal DA-9®, and an oil having a Specific Gravity between 0.90 and 0.95.

Another embodiment comprises cyclosporin A, Chemal LA-23®, and an oil having a Specific Gravity between 0.90 and 0.95.

Another embodiment comprises cyclosporin A, Chemal LA-4®, and an oil having a Specific Gravity between 0.90 and 0.95.

Another embodiment comprises cyclosporin A, Chemal LA-9®, and an oil having a Specific Gravity between 0.90 and 0.95.

Another embodiment comprises cyclosporin A, Chemal OA-20G®, and an oil having a Specific Gravity between 0.90 and 0.95.

Another embodiment comprises cyclosporin A, Chemal OA-5®, and an oil having a Specific Gravity between 0.90 and 0.95.

Another embodiment comprises cyclosporin A, Chemal OA-9®, and an oil having a Specific Gravity between 0.90 and 0.95.

Another embodiment comprises cyclosporin A, Chemal TDA-12®, and an oil having a Specific Gravity between 0.90 and 0.95.

Another embodiment comprises cyclosporin A, Chemal TDA-15®, and an oil having a Specific Gravity between 0.90 and 0.95.

Another embodiment comprises cyclosporin A, Chemal TDA-3®, and an oil having a Specific Gravity between 0.90 and 0.95.

Another embodiment comprises cyclosporin A, Chemal TDA-6®, and an oil having a Specific Gravity between 0.90 and 0.95.

Another embodiment comprises cyclosporin A, Chemal TDA-9®, and an oil having a Specific Gravity between 0.90 and 0.95.

Another embodiment comprises cyclosporin A, Chemax CO-16®, and an oil having a Specific Gravity between 0.90 and 0.95.

Another embodiment comprises cyclosporin A, Chemax CO-200/50®, and an oil having a Specific Gravity between 0.90 and 0.95.

Another embodiment comprises cyclosporin A, Chemax CO-25®, and an oil having a Specific Gravity between 0.90 and 0.95.

Another embodiment comprises cyclosporin A, Chemax CO-30®, and an oil having a Specific Gravity between 0.90 and 0.95.

Another embodiment comprises cyclosporin A, Chemax CO-36®, and an oil having a Specific Gravity between 0.90 and 0.95.

Another embodiment comprises cyclosporin A, Chemax CO-40®, and an oil having a Specific Gravity between 0.90 and 0.95.

Another embodiment comprises cyclosporin A, Chemax CO-5®, and an oil having a Specific Gravity between 0.90 and 0.95.

Another embodiment comprises cyclosporin A, Chemax CO-80®, and an oil having a Specific Gravity between 0.90 and 0.95.

Another embodiment comprises cyclosporin A, Chemax DNP-150/50®, and an oil having a Specific Gravity between 0.90 and 0.95.

Another embodiment comprises cyclosporin A, Chemax DNP-18®, and an oil having a Specific Gravity between 0.90 and 0.95.

Another embodiment comprises cyclosporin A, Chemax DNP-8®, and an oil having a Specific Gravity between 0.90 and 0.95.

Another embodiment comprises cyclosporin A, Chemax E 200 ML®, and an oil having a Specific Gravity between 0.90 and 0.95.

Another embodiment comprises cyclosporin A, Chemax E 200 MO®, and an oil having a Specific Gravity between 0.90 and 0.95.

Another embodiment comprises cyclosporin A, Chemax E 400 ML®, and an oil having a Specific Gravity between 0.90 and 0.95.

Another embodiment comprises cyclosporin A, Chemax E 400 MO®, and an oil having a Specific Gravity between 0.90 and 0.95.

Another embodiment comprises cyclosporin A, Chemax E 600 ML®, and an oil having a Specific Gravity between 0.90 and 0.95.

Another embodiment comprises cyclosporin A, Chemax E 600 MO®, and an oil having a Specific Gravity between 0.90 and 0.95.

Another embodiment comprises cyclosporin A, Chemax E1000 MO®, and an oil having a Specific Gravity between 0.90 and 0.95.

Another embodiment comprises cyclosporin A, Chemax E1000 MS®, and an oil having a Specific Gravity between 0.90 and 0.95.

Another embodiment comprises cyclosporin A, Chemax E200 MS®, and an oil having a Specific Gravity between 0.90 and 0.95.

Another embodiment comprises cyclosporin A, Chemax E400 MS®, and an oil having a Specific Gravity between 0.90 and 0.95.

Another embodiment comprises cyclosporin A, Chemax E600 MS®, and an oil having a Specific Gravity between 0.90 and 0.95.

Another embodiment comprises cyclosporin A, Chemax HCO-16®, and an oil having a Specific Gravity between 0.90 and 0.95.

Another embodiment comprises cyclosporin A, Chemax HCO-200/50®, and an oil having a Specific Gravity between 0.90 and 0.95.

Another embodiment comprises cyclosporin A, Chemax HCO-25®, and an oil having a Specific Gravity between 0.90 and 0.95.

Another embodiment comprises cyclosporin A, Chemax HCO-5®, and an oil having a Specific Gravity between 0.90 and 0.95.

Another embodiment comprises cyclosporin A, Chemax TO-16®, and an oil having a Specific Gravity between 0.90 and 0.95.

Another embodiment comprises cyclosporin A, Chemax TO-8®, and an oil having a Specific Gravity between 0.90 and 0.95.

Another embodiment comprises cyclosporin A, Colalube 3409®, and an oil having a Specific Gravity between 0.90 and 0.95.

Another embodiment comprises cyclosporin A, Colalube 3411®, and an oil having a Specific Gravity between 0.90 and 0.95.

Another embodiment comprises cyclosporin A, Colalube 3413®, and an oil having a Specific Gravity between 0.90 and 0.95.

Another embodiment comprises cyclosporin A, Colalube 3414®, and an oil having a Specific Gravity between 0.90 and 0.95.

Another embodiment comprises cyclosporin A, Colalube 3415®, and an oil having a Specific Gravity between 0.90 and 0.95.

Another embodiment comprises cyclosporin A, Colalube 3416®, and an oil having a Specific Gravity between 0.90 and 0.95.

Another embodiment comprises cyclosporin A, Colalube 3417®, and an oil having a Specific Gravity between 0.90 and 0.95.

Another embodiment comprises cyclosporin A, Delonic NPE-100®, and an oil having a Specific Gravity between 0.90 and 0.95.

Another embodiment comprises cyclosporin A, Delonic NPE-30®, and an oil having a Specific Gravity between 0.90 and 0.95.

Another embodiment comprises cyclosporin A, Delonic NPE-40®, and an oil having a Specific Gravity between 0.90 and 0.95.

Another embodiment comprises cyclosporin A, Delonic OPE-10®, and an oil having a Specific Gravity between 0.90 and 0.95.

Another embodiment comprises cyclosporin A, Delonic OPE-12®, and an oil having a Specific Gravity between 0.90 and 0.95.

Another embodiment comprises cyclosporin A, Delonic OPE-30®, and an oil having a Specific Gravity between 0.90 and 0.95.

Another embodiment comprises cyclosporin A, Delonic OPE-40®, and an oil having a Specific Gravity between 0.90 and 0.95.

Another embodiment comprises cyclosporin A, Delonic OPE-5®, and an oil having a Specific Gravity between 0.90 and 0.95.

Another embodiment comprises cyclosporin A, Delonic OPE-7.5®, and an oil having a Specific Gravity between 0.90 and 0.95.

Another embodiment comprises cyclosporin A, DePeg 16-CO®, and an oil having a Specific Gravity between 0.90 and 0.95.

Another embodiment comprises cyclosporin A, DePeg 25-CO®, and an oil having a Specific Gravity between 0.90 and 0.95.

Another embodiment comprises cyclosporin A, DePeg 30-CO®, and an oil having a Specific Gravity between 0.90 and 0.95.

Another embodiment comprises cyclosporin A, DePeg 40-CO®, and an oil having a Specific Gravity between 0.90 and 0.95.

Another embodiment comprises cyclosporin A, DePeg 5-CO®, and an oil having a Specific Gravity between 0.90 and 0.95.

Another embodiment comprises cyclosporin A, DePeg 80-CO®, and an oil having a Specific Gravity between 0.90 and 0.95.

Another embodiment comprises cyclosporin A, DeSonic S-100®, and an oil having a Specific Gravity between 0.90 and 0.95.

Another embodiment comprises cyclosporin A, DeSonic S-114®, and an oil having a Specific Gravity between 0.90 and 0.95.

Another embodiment comprises cyclosporin A, DeSonic S-405®, and an oil having a Specific Gravity between 0.90 and 0.95.

Another embodiment comprises cyclosporin A, DeSonic S-45®, and an oil having a Specific Gravity between 0.90 and 0.95.

Another embodiment comprises cyclosporin A, DeSonic TDA-9®, and an oil having a Specific Gravity between 0.90 and 0.95.

Another embodiment comprises cyclosporin A, DeThox Acid O-14®, and an oil having a Specific Gravity between 0.90 and 0.95.

Another embodiment comprises cyclosporin A, DeThox Acid O-16®, and an oil having a Specific Gravity between 0.90 and 0.95.

Another embodiment comprises cyclosporin A, DeThox Acid O-9®, and an oil having a Specific Gravity between 0.90 and 0.95.

Another embodiment comprises cyclosporin A, DeThox Acid TO-14®, and an oil having a Specific Gravity between 0.90 and 0.95.

Another embodiment comprises cyclosporin A, DeThox Acid TO-16.5®, and an oil having a Specific Gravity between 0.90 and 0.95.

Another embodiment comprises cyclosporin A, DeThox Acid TO-8.5®, and an oil having a Specific Gravity between 0.90 and 0.95.

Another embodiment comprises cyclosporin A, Dur-Em 114®, and an oil having a Specific Gravity between 0.90 and 0.95.

Another embodiment comprises cyclosporin A, Dur-Em 117®, and an oil having a Specific Gravity between 0.90 and 0.95.

Another embodiment comprises cyclosporin A, Dur-Em 204®, and an oil having a Specific Gravity between 0.90 and 0.95.

Another embodiment comprises cyclosporin A, Dur-Em 207®, and an oil having a Specific Gravity between 0.90 and 0.95.

Another embodiment comprises cyclosporin A, Dur-Em 207E®, and an oil having a Specific Gravity between 0.90 and 0.95.

Another embodiment comprises cyclosporin A, Emerest 2704®, and an oil having a Specific Gravity between 0.90 and 0.95.

Another embodiment comprises cyclosporin A, Emerest 2712®, and an oil having a Specific Gravity between 0.90 and 0.95.

Another embodiment comprises cyclosporin A, Emerest 2715®, and an oil having a Specific Gravity between 0.90 and 0.95.

Another embodiment comprises cyclosporin A, Ethal EH-2®, and an oil having a Specific Gravity between 0.90 and 0.95.

Another embodiment comprises cyclosporin A, Ethal EH-5®, and an oil having a Specific Gravity between 0.90 and 0.95.

Another embodiment comprises cyclosporin A, Ethal OA-23®, and an oil having a Specific Gravity between 0.90 and 0.95.

Another embodiment comprises cyclosporin A, Ethosperse CA-20®, and an oil having a Specific Gravity between 0.90 and 0.95.

Another embodiment comprises cyclosporin A, Ethosperse LA-12®, and an oil having a Specific Gravity between 0.90 and 0.95.

Another embodiment comprises cyclosporin A, Ethosperse LA-23®, and an oil having a Specific Gravity between 0.90 and 0.95.

Another embodiment comprises cyclosporin A, Ethosperse LA-4®, and an oil having a Specific Gravity between 0.90 and 0.95.

Another embodiment comprises cyclosporin A, Ethox 3095®, and an oil having a Specific Gravity between 0.90 and 0.95.

Another embodiment comprises cyclosporin A, Ethox CO-16®, and an oil having a Specific Gravity between 0.90 and 0.95.

Another embodiment comprises cyclosporin A, Ethox CO-200®, and an oil having a Specific Gravity between 0.90 and 0.95.

Another embodiment comprises cyclosporin A, Ethox CO-25®, and an oil having a Specific Gravity between 0.90 and 0.95.

Another embodiment comprises cyclosporin A, Ethox CO-30®, and an oil having a Specific Gravity between 0.90 and 0.95.

Another embodiment comprises cyclosporin A, Ethox CO-36®, and an oil having a Specific Gravity between 0.90 and 0.95.

Another embodiment comprises cyclosporin A, Ethox CO-40®, and an oil having a Specific Gravity between 0.90 and 0.95.

Another embodiment comprises cyclosporin A, Ethox CO-5®, and an oil having a Specific Gravity between 0.90 and 0.95.

Another embodiment comprises cyclosporin A, Ethox DO-14®, and an oil having a Specific Gravity between 0.90 and 0.95.

Another embodiment comprises cyclosporin A, Ethox DO-9®, and an oil having a Specific Gravity between 0.90 and 0.95.

Another embodiment comprises cyclosporin A, Ethox MA-8®, and an oil having a Specific Gravity between 0.90 and 0.95.

Another embodiment comprises cyclosporin A, Ethox MI-14®, and an oil having a Specific Gravity between 0.90 and 0.95.

Another embodiment comprises cyclosporin A, Ethox ML-14®, and an oil having a Specific Gravity between 0.90 and 0.95.

Another embodiment comprises cyclosporin A, Ethox ML-5®, and an oil having a Specific Gravity between 0.90 and 0.95.

Another embodiment comprises cyclosporin A, Ethox MO-14®, and an oil having a Specific Gravity between 0.90 and 0.95.

Another embodiment comprises cyclosporin A, Ethox MO-9®, and an oil having a Specific Gravity between 0.90 and 0.95.

Another embodiment comprises cyclosporin A, Ethox MS-14®, and an oil having a Specific Gravity between 0.90 and 0.95.

Another embodiment comprises cyclosporin A, Ethox MS-23®, and an oil having a Specific Gravity between 0.90 and 0.95.

Another embodiment comprises cyclosporin A, Ethox MS-40®, and an oil having a Specific Gravity between 0.90 and 0.95.

Another embodiment comprises cyclosporin A, Ethox MS-8®, and an oil having a Specific Gravity between 0.90 and 0.95.

Another embodiment comprises cyclosporin A, Ethox TO-16®, and an oil having a Specific Gravity between 0.90 and 0.95.

Another embodiment comprises cyclosporin A, Ethox TO-8®, and an oil having a Specific Gravity between 0.90 and 0.95.

Another embodiment comprises cyclosporin A, Ethylan SN-120®, and an oil having a Specific Gravity between 0.90 and 0.95.

Another embodiment comprises cyclosporin A, Ethylan SN-70®, and an oil having a Specific Gravity between 0.90 and 0.95.

Another embodiment comprises cyclosporin A, Ethylan SN-90®, and an oil having a Specific Gravity between 0.90 and 0.95.

Another embodiment comprises cyclosporin A, Flexricin 13®, and an oil having a Specific Gravity between 0.90 and 0.95.

Another embodiment comprises cyclosporin A, Flexricin 15®, and an oil having a Specific Gravity between 0.90 and 0.95.

Another embodiment comprises cyclosporin A, Genapol 1261®, and an oil having a Specific Gravity between 0.90 and 0.95.

Another embodiment comprises cyclosporin A, Generol 122 E25®, and an oil having a Specific Gravity between 0.90 and 0.95.

Another embodiment comprises cyclosporin A, Glicepol 160®, and an oil having a Specific Gravity between 0.90 and 0.95.

Another embodiment comprises cyclosporin A, Glicepol 560®, and an oil having a Specific Gravity between 0.90 and 0.95.

Another embodiment comprises cyclosporin A, Glicepol GMS 20®, and an oil having a Specific Gravity between 0.90 and 0.95.

Another embodiment comprises cyclosporin A, Hetoxide BN-13®, and an oil having a Specific Gravity between 0.90 and 0.95.

Another embodiment comprises cyclosporin A, Hetoxide DNP-4®, and an oil having a Specific Gravity between 0.90 and 0.95.

Another embodiment comprises cyclosporin A, Hetoxide DNP-9.6®, and an oil having a Specific Gravity between 0.90 and 0.95.

Another embodiment comprises cyclosporin A, Hetoxide G-26®, and an oil having a Specific Gravity between 0.90 and 0.95.

Another embodiment comprises cyclosporin A, Hetoxide G-7®, and an oil having a Specific Gravity between 0.90 and 0.95.

Another embodiment comprises cyclosporin A, Hetoxide GT-20®, and an oil having a Specific Gravity between 0.90 and 0.95.

Another embodiment comprises cyclosporin A, Hetoxide HC-40®, and an oil having a Specific Gravity between 0.90 and 0.95.

Another embodiment comprises cyclosporin A, Hetoxol C-24®, and an oil having a Specific Gravity between 0.90 and 0.95.

Another embodiment comprises cyclosporin A, Hetoxol CA-10®, and an oil having a Specific Gravity between 0.90 and 0.95.

Another embodiment comprises cyclosporin A, Hetoxol CA-2®, and an oil having a Specific Gravity between 0.90 and 0.95.

Another embodiment comprises cyclosporin A, Hetoxol CA-20®, and an oil having a Specific Gravity between 0.90 and 0.95.

Another embodiment comprises cyclosporin A, Hetoxol CAWS®, and an oil having a Specific Gravity between 0.90 and 0.95.

Another embodiment comprises cyclosporin A, Hetoxol CD-3®, and an oil having a Specific Gravity between 0.90 and 0.95.

Another embodiment comprises cyclosporin A, Hetoxol CD-4®, and an oil having a Specific Gravity between 0.90 and 0.95.

Another embodiment comprises cyclosporin A, Hetoxol CS-15®, and an oil having a Specific Gravity between 0.90 and 0.95.

Another embodiment comprises cyclosporin A, Hetoxol CS-20®, and an oil having a Specific Gravity between 0.90 and 0.95.

Another embodiment comprises cyclosporin A, Hetoxol CS-20D®, and an oil having a Specific Gravity between 0.90 and 0.95.

Another embodiment comprises cyclosporin A, Hetoxol CS-25®, and an oil having a Specific Gravity between 0.90 and 0.95.

Another embodiment comprises cyclosporin A, Hetoxol CS-50®, and an oil having a Specific Gravity between 0.90 and 0.95.

Another embodiment comprises cyclosporin A, Hetoxol CS-9®, and an oil having a Specific Gravity between 0.90 and 0.95.

Another embodiment comprises cyclosporin A, Hetoxol CSA-15®, and an oil having a Specific Gravity between 0.90 and 0.95.

Another embodiment comprises cyclosporin A, Hetoxol D®, and an oil having a Specific Gravity between 0.90 and 0.95.

Another embodiment comprises cyclosporin A, Hetoxol G®, and an oil having a Specific Gravity between 0.90 and 0.95.

Another embodiment comprises cyclosporin A, Hetoxol J®, and an oil having a Specific Gravity between 0.90 and 0.95.

Another embodiment comprises cyclosporin A, Hetoxol L®, and an oil having a Specific Gravity between 0.90 and 0.95.

Another embodiment comprises cyclosporin A, Hetoxol L-23®, and an oil having a Specific Gravity between 0.90 and 0.95.

Another embodiment comprises cyclosporin A, Hetoxol L-4®, and an oil having a Specific Gravity between 0.90 and 0.95.

Another embodiment comprises cyclosporin A, Hetoxol L-9®, and an oil having a Specific Gravity between 0.90 and 0.95.

Another embodiment comprises cyclosporin A, Hetoxol LS-9®, and an oil having a Specific Gravity between 0.90 and 0.95.

Another embodiment comprises cyclosporin A, Hetoxol M-3®, and an oil having a Specific Gravity between 0.90 and 0.95.

Another embodiment comprises cyclosporin A, Hetoxol OA-10 Special®, and an oil having a Specific Gravity between 0.90 and 0.95.

Another embodiment comprises cyclosporin A, Hetoxol OA-20 Special®, and an oil having a Specific Gravity between 0.90 and 0.95.

Another embodiment comprises cyclosporin A, Hetoxol OA-3 Special®, and an oil having a Specific Gravity between 0.90 and 0.95.

Another embodiment comprises cyclosporin A, Hetoxol OA-5 Special®, and an oil having a Specific Gravity between 0.90 and 0.95.

Another embodiment comprises cyclosporin A, Hetoxol OL-10®, and an oil having a Specific Gravity between 0.90 and 0.95.

Another embodiment comprises cyclosporin A, Hetoxol OL-2®, and an oil having a Specific Gravity between 0.90 and 0.95.

Another embodiment comprises cyclosporin A, Hetoxol OL-23®, and an oil having a Specific Gravity between 0.90 and 0.95.

Another embodiment comprises cyclosporin A, Hetoxol OL-4®, and an oil having a Specific Gravity between 0.90 and 0.95.

Another embodiment comprises cyclosporin A, Hetoxol OL-40®, and an oil having a Specific Gravity between 0.90 and 0.95.

Another embodiment comprises cyclosporin A, Hetoxol OL-5®, and an oil having a Specific Gravity between 0.90 and 0.95.

Another embodiment comprises cyclosporin A, Hetoxol PLA®, and an oil having a Specific Gravity between 0.90 and 0.95.

Another embodiment comprises cyclosporin A, Hetoxol SP-15®, and an oil having a Specific Gravity between 0.90 and 0.95.

Another embodiment comprises cyclosporin A, Hetoxol STA-10®, and an oil having a Specific Gravity between 0.90 and 0.95.

Another embodiment comprises cyclosporin A, Hetoxol STA-2®, and an oil having a Specific Gravity between 0.90 and 0.95.

Another embodiment comprises cyclosporin A, Hetoxol STA-30®, and an oil having a Specific Gravity between 0.90 and 0.95.

Another embodiment comprises cyclosporin A, Hetoxol TD-12®, and an oil having a Specific Gravity between 0.90 and 0.95.

Another embodiment comprises cyclosporin A, Hetoxol TD-18®, and an oil having a Specific Gravity between 0.90 and 0.95.

Another embodiment comprises cyclosporin A, Hetoxol TD-3®, and an oil having a Specific Gravity between 0.90 and 0.95.

Another embodiment comprises cyclosporin A, Hetoxol TD-6®, and an oil having a Specific Gravity between 0.90 and 0.95.

Another embodiment comprises cyclosporin A, Hetoxol TD-9®, and an oil having a Specific Gravity between 0.90 and 0.95.

Another embodiment comprises cyclosporin A, Hetoxol TDEP-15®, and an oil having a Specific Gravity between 0.90 and 0.95.

Another embodiment comprises cyclosporin A, Hetoxol TDEP-63®, and an oil having a Specific Gravity between 0.90 and 0.95.

Another embodiment comprises cyclosporin A, Iconol 24-12®, and an oil having a Specific Gravity between 0.90 and 0.95.

Another embodiment comprises cyclosporin A, Iconol 24-23®, and an oil having a Specific Gravity between 0.90 and 0.95.

Another embodiment comprises cyclosporin A, Iconol 24-4®, and an oil having a Specific Gravity between 0.90 and 0.95.

Another embodiment comprises cyclosporin A, Iconol DA-4®, and an oil having a Specific Gravity between 0.90 and 0.95.

Another embodiment comprises cyclosporin A, Iconol DA-6®, and an oil having a Specific Gravity between 0.90 and 0.95.

Another embodiment comprises cyclosporin A, Iconol TDA-10®, and an oil having a Specific Gravity between 0.90 and 0.95.

Another embodiment comprises cyclosporin A, Iconol TDA-3®, and an oil having a Specific Gravity between 0.90 and 0.95.

Another embodiment comprises cyclosporin A, Iconol TDA-6®, and an oil having a Specific Gravity between 0.90 and 0.95.

Another embodiment comprises cyclosporin A, Iconol TDA-8-90%®, and an oil having a Specific Gravity between 0.90 and 0.95.

Another embodiment comprises cyclosporin A, Iconol TDA-9®, and an oil having a Specific Gravity between 0.90 and 0.95.

Another embodiment comprises cyclosporin A, Igepal CA-210®, and an oil having a Specific Gravity between 0.90 and 0.95.

Another embodiment comprises cyclosporin A, Igepal CA-407®, and an oil having a Specific Gravity between 0.90 and 0.95.

Another embodiment comprises cyclosporin A, Igepal CA-420®, and an oil having a Specific Gravity between 0.90 and 0.95.

Another embodiment comprises cyclosporin A, Igepal CA-520®, and an oil having a Specific Gravity between 0.90 and 0.95.

Another embodiment comprises cyclosporin A, Igepal CA-620®, and an oil having a Specific Gravity between 0.90 and 0.95.

Another embodiment comprises cyclosporin A, Igepal CA-630®, and an oil having a Specific Gravity between 0.90 and 0.95.

Another embodiment comprises cyclosporin A, Igepal CA-720®, and an oil having a Specific Gravity between 0.90 and 0.95.

Another embodiment comprises cyclosporin A, Igepal CA-877®, and an oil having a Specific Gravity between 0.90 and 0.95.

Another embodiment comprises cyclosporin A, Igepal CA-887®, and an oil having a Specific Gravity between 0.90 and 0.95.

Another embodiment comprises cyclosporin A, Igepal CA-897®, and an oil having a Specific Gravity between 0.90 and 0.95.

Another embodiment comprises cyclosporin A, Igepal CO-210®, and an oil having a Specific Gravity between 0.90 and 0.95.

Another embodiment comprises cyclosporin A, Igepal CO-430®, and an oil having a Specific Gravity between 0.90 and 0.95.

Another embodiment comprises cyclosporin A, Igepal CO-520®, and an oil having a Specific Gravity between 0.90 and 0.95.

Another embodiment comprises cyclosporin A, Igepal CO-530®, and an oil having a Specific Gravity between 0.90 and 0.95.

Another embodiment comprises cyclosporin A, Igepal CO-610®, and an oil having a Specific Gravity between 0.90 and 0.95.

Another embodiment comprises cyclosporin A, Igepal CO-630®, and an oil having a Specific Gravity between 0.90 and 0.95.

Another embodiment comprises cyclosporin A, Igepal CO-630 Special®, and an oil having a Specific Gravity between 0.90 and 0.95.

Another embodiment comprises cyclosporin A, Igepal CO-660®, and an oil having a Specific Gravity between 0.90 and 0.95.

Another embodiment comprises cyclosporin A, Igepal CO-710®, and an oil having a Specific Gravity between 0.90 and 0.95.

Another embodiment comprises cyclosporin A, Igepal CO-720®, and an oil having a Specific Gravity between 0.90 and 0.95.

Another embodiment comprises cyclosporin A, Igepal CO-730®, and an oil having a Specific Gravity between 0.90 and 0.95.

Another embodiment comprises cyclosporin A, Igepal CO-738®, and an oil having a Specific Gravity between 0.90 and 0.95.

Another embodiment comprises cyclosporin A, Igepal GO-850®, and an oil having a Specific Gravity between 0.90 and 0.95.

Another embodiment comprises cyclosporin A, Igepal CO-880®, and an oil having a Specific Gravity between 0.90 and 0.95.

Another embodiment comprises cyclosporin A, Igepal CO-887®, and an oil having a Specific Gravity between 0.90 and 0.95.

Another embodiment comprises cyclosporin A, Igepal CO-890®, and an oil having a Specific Gravity between 0.90 and 0.95.

Another embodiment comprises cyclosporin A, Igepal GO-897®, and an oil having a Specific Gravity between 0.90 and 0.95.

Another embodiment comprises cyclosporin A, Igepal CO-970®, and an oil having a Specific Gravity between 0.90 and 0.95.

Another embodiment comprises cyclosporin A, Igepal CO-977®, and an oil having a Specific Gravity between 0.90 and 0.95.

Another embodiment comprises cyclosporin A, Igepal CO-987®, and an oil having a Specific Gravity between 0.90 and 0.95.

Another embodiment comprises cyclosporin A, Igepal CO-990 FLK®, and an oil having a Specific Gravity between 0.90 and 0.95.

Another embodiment comprises cyclosporin A, Igepal CO-997®, and an oil having a Specific Gravity between 0.90 and 0.95.

Another embodiment comprises cyclosporin A, Igepal DM-430®, and an oil having a Specific Gravity between 0.90 and 0.95.

Another embodiment comprises cyclosporin A, Igepal DM-530®, and an oil having a Specific Gravity between 0.90 and 0.95.

Another embodiment comprises cyclosporin A, Igepal DM-710®, and an oil having a Specific Gravity between 0.90 and 0.95.

Another embodiment comprises cyclosporin A, Igepal DM-730®, and an oil having a Specific Gravity between 0.90 and 0.95.

Another embodiment comprises cyclosporin A, Igepal DM-880®, and an oil having a Specific Gravity between 0.90 and 0.95.

Another embodiment comprises cyclosporin A, Igepal DM-970 FLK®, and an oil having a Specific Gravity between 0.90 and 0.95.

Another embodiment comprises cyclosporin A, Igepal OD-410®, and an oil having a Specific Gravity between 0.90 and 0.95.

Another embodiment comprises cyclosporin A, Igepal RC-520®, and an oil having a Specific Gravity between 0.90 and 0.95.

Another embodiment comprises cyclosporin A, Igepal RC-620®, and an oil having a Specific Gravity between 0.90 and 0.95.

Another embodiment comprises cyclosporin A, Igepal RC-630®, and an oil having a Specific Gravity between 0.90 and 0.95.

Another embodiment comprises cyclosporin A, Lexemul CS-20®, and an oil having a Specific Gravity between 0.90 and 0.95.

Another embodiment comprises cyclosporin A, Lipo GMS 450®, and an oil having a Specific Gravity between 0.90 and 0.95.

Another embodiment comprises cyclosporin A, Lipo Polyglycol 1000®, and an oil having a Specific Gravity between 0.90 and 0.95.

Another embodiment comprises cyclosporin A, Lipo Polyglycol 200®, and an oil having a Specific Gravity between 0.90 and 0.95.

Another embodiment comprises cyclosporin A, Lipo Polyglycol 300®, and an oil having a Specific Gravity between 0.90 and 0.95.

Another embodiment comprises cyclosporin A, Lipo Polyglycol 3350®, and an oil having a Specific Gravity between 0.90 and 0.95.

Another embodiment comprises cyclosporin A, Lipo Polyglycol 400®, and an oil having a Specific Gravity between 0.90 and 0.95.

Another embodiment comprises cyclosporin A, Lipo Polyglycol 600®, and an oil having a Specific Gravity between 0.90 and 0.95.

Another embodiment comprises cyclosporin A, Lipocol C-10®, and an oil having a Specific Gravity between 0.90 and 0.95.

Another embodiment comprises cyclosporin A, Lipocol C-2®, and an oil having a Specific Gravity between 0.90 and 0.95.

Another embodiment comprises cyclosporin A, Lipocol C-20®, and an oil having a Specific Gravity between 0.90 and 0.95.

Another embodiment comprises cyclosporin A, Lipocol L-12®, and an oil having a Specific Gravity between 0.90 and 0.95.

Another embodiment comprises cyclosporin A, Lipocol L-23®, and an oil having a Specific Gravity between 0.90 and 0.95.

Another embodiment comprises cyclosporin A, Lipocol L-4®, and an oil having a Specific Gravity between 0.90 and 0.95.

Another embodiment comprises cyclosporin A, Lipocol O-10®, and an oil having a Specific Gravity between 0.90 and 0.95.

Another embodiment comprises cyclosporin A, Lipocol O-2®, and an oil having a Specific Gravity between 0.90 and 0.95.

Another embodiment comprises cyclosporin A, Lipocol O-20®, and an oil having a Specific Gravity between 0.90 and 0.95.

Another embodiment comprises cyclosporin A, Lipocol O-5®, and an oil having a Specific Gravity between 0.90 and 0.95.

Another embodiment comprises cyclosporin A, Lipocol S-10®, and an oil having a Specific Gravity between 0.90 and 0.95.

Another embodiment comprises cyclosporin A, Lipocol S-2®, and an oil having a Specific Gravity between 0.90 and 0.95.

Another embodiment comprises cyclosporin A, Lipocol S-20®, and an oil having a Specific Gravity between 0.90 and 0.95.

Another embodiment comprises cyclosporin A, Lipocol SC-15®, and an oil having a Specific Gravity between 0.90 and 0.95.

Another embodiment comprises cyclosporin A, Lipocol SC-20®, and an oil having a Specific Gravity between 0.90 and 0.95.

Another embodiment comprises cyclosporin A, Lipocol SC-4®, and an oil having a Specific Gravity between 0.90 and 0.95.

Another embodiment comprises cyclosporin A, Lipocol TD-12®, and an oil having a Specific Gravity between 0.90 and 0.95.

Another embodiment comprises cyclosporin A, Lumulse 40-O K®, and an oil having a Specific Gravity between 0.90 and 0.95.

Another embodiment comprises cyclosporin A, Lumulse 42-O K®, and an oil having a Specific Gravity between 0.90 and 0.95.

Another embodiment comprises cyclosporin A, Lumulse GMT-40®, and an oil having a Specific Gravity between 0.90 and 0.95.

Another embodiment comprises cyclosporin A, Lumulse PEG 1450®, and an oil having a Specific Gravity between 0.90 and 0.95.

Another embodiment comprises cyclosporin A, Lumulse PEG 1450 NF®, and an oil having a Specific Gravity between 0.90 and 0.95.

Another embodiment comprises cyclosporin A, Lumulse PEG 8000®, and an oil having a Specific Gravity between 0.90 and 0.95.

Another embodiment comprises cyclosporin A, Lumulse PGO®, and an oil having a Specific Gravity between 0.90 and 0.95.

Another embodiment comprises cyclosporin A, Lumulse POE (2) Oleyl Amine®, and an oil having a Specific Gravity between 0.90 and 0.95.

Another embodiment comprises cyclosporin A, Magrabar PGO-104®, and an oil having a Specific Gravity between 0.90 and 0.95.

Another embodiment comprises cyclosporin A, Mapeg 200 ML®, and an oil having a Specific Gravity between 0.90 and 0.95.

Another embodiment comprises cyclosporin A, Mapeg 400 MO®, and an oil having a Specific Gravity between 0.90 and 0.95.

Another embodiment comprises cyclosporin A, Mapeg 400 MOT®, and an oil having a Specific Gravity between 0.90 and 0.95.

Another embodiment comprises cyclosporin A, Mapeg 400DO®, and an oil having a Specific Gravity between 0.90 and 0.95.

Another embodiment comprises cyclosporin A, Mapeg 400DOT®, and an oil having a Specific Gravity between 0.90 and 0.95.

Another embodiment comprises cyclosporin A, Mapeg 600DOT®, and an oil having a Specific Gravity between 0.90 and 0.95.

Another embodiment comprises cyclosporin A, Marlox B 24/80®, and an oil having a Specific Gravity between 0.90 and 0.95.

Another embodiment comprises cyclosporin A, Marlox FK 86®, and an oil having a Specific Gravity between 0.90 and 0.95.

Another embodiment comprises cyclosporin A, Marlox MO 154®, and an oil having a Specific Gravity between 0.90 and 0.95.

Another embodiment comprises cyclosporin A, Mazox CDA CG®, and an oil having a Specific Gravity between 0.90 and 0.95.

Another embodiment comprises cyclosporin A, Monoglycerides Glyceryl Monestearate®, and an oil having a Specific Gravity between 0.90 and 0.95.

Another embodiment comprises cyclosporin A, Myrj 45®, and an oil having a Specific Gravity between 0.90 and 0.95.

Another embodiment comprises cyclosporin A, Myrj 52®, and an oil having a Specific Gravity between 0.90 and 0.95.

Another embodiment comprises cyclosporin A, Myrj 52S®, and an oil having a Specific Gravity between 0.90 and 0.95.

Another embodiment comprises cyclosporin A, Myrj 53®, and an oil having a Specific Gravity between 0.90 and 0.95.

Another embodiment comprises cyclosporin A, Myrj 59®, and an oil having a Specific Gravity between 0.90 and 0.95.

Another embodiment comprises cyclosporin A, Norfox NP-12®, and an oil having a Specific Gravity between 0.90 and 0.95.

Another embodiment comprises cyclosporin A, Norfox NP-4®, and an oil having a Specific Gravity between 0.90 and 0.95.

Another embodiment comprises cyclosporin A, Norfox NP-6®, and an oil having a Specific Gravity between 0.90 and 0.95.

Another embodiment comprises cyclosporin A, Norfox NP-887®, and an oil having a Specific Gravity between 0.90 and 0.95.

Another embodiment comprises cyclosporin A, Norfox NP-9®, and an oil having a Specific Gravity between 0.90 and 0.95.

Another embodiment comprises cyclosporin A, Norfox NP-977®, and an oil having a Specific Gravity between 0.90 and 0.95.

Another embodiment comprises cyclosporin A, Norfox OP-100®, and an oil having a Specific Gravity between 0.90 and 0.95.

Another embodiment comprises cyclosporin A, Norfox OP-102®, and an oil having a Specific Gravity between 0.90 and 0.95.

Another embodiment comprises cyclosporin A, Norfox OP-114®, and an oil having a Specific Gravity between 0.90 and 0.95.

Another embodiment comprises cyclosporin A, Norfox OP-45®, and an oil having a Specific Gravity between 0.90 and 0.95.

Another embodiment comprises cyclosporin A, Pegosperse 100-L®, and an oil having a Specific Gravity between 0.90 and 0.95.

Another embodiment comprises cyclosporin A, Pegosperse 100-O®, and an oil having a Specific Gravity between 0.90 and 0.95.

Another embodiment comprises cyclosporin A, Pegosperse 100-S®, and an oil having a Specific Gravity between 0.90 and 0.95.

Another embodiment comprises cyclosporin A, Pegosperse 1500-MS®, and an oil having a Specific Gravity between 0.90 and 0.95.

Another embodiment comprises cyclosporin A, Pegosperse 1750-MS®, and an oil having a Specific Gravity between 0.90 and 0.95.

Another embodiment comprises cyclosporin A, Pegosperse 200 DL®, and an oil having a Specific Gravity between 0.90 and 0.95.

Another embodiment comprises cyclosporin A, Pegosperse 200-ML®, and an oil having a Specific Gravity between 0.90 and 0.95.

Another embodiment comprises cyclosporin A, Pegosperse 400-DL®, and an oil having a Specific Gravity between 0.90 and 0.95.

Another embodiment comprises cyclosporin A, Pegosperse 400-DO®, and an oil having a Specific Gravity between 0.90 and 0.95.

Another embodiment comprises cyclosporin A, Pegosperse 400-DS®, and an oil having a Specific Gravity between 0.90 and 0.95.

Another embodiment comprises cyclosporin A, Pegosperse 400-ML®, and an oil having a Specific Gravity between 0.90 and 0.95.

Another embodiment comprises cyclosporin A, Pegosperse 400-MO®, and an oil having a Specific Gravity between 0.90 and 0.95.

Another embodiment comprises cyclosporin A, Pegosperse 400-MOT®, and an oil having a Specific Gravity between 0.90 and 0.95.

Another embodiment comprises cyclosporin A, Pegosperse 400-MS®, and an oil having a Specific Gravity between 0.90 and 0.95.

Another embodiment comprises cyclosporin A, Pegosperse 50-DS®, and an oil having a Specific Gravity between 0.90 and 0.95.

Another embodiment comprises cyclosporin A, Pegosperse 50-MS®, and an oil having a Specific Gravity between 0.90 and 0.95.

Another embodiment comprises cyclosporin A, Pegosperse 600-ML®, and an oil having a Specific Gravity between 0.90 and 0.95.

Another embodiment comprises cyclosporin A, Pegosperse 600-MS®, and an oil having a Specific Gravity between 0.90 and 0.95.

Another embodiment comprises cyclosporin A, Plurafac A-38®, and an oil having a Specific Gravity between 0.90 and 0.95.

Another embodiment comprises cyclosporin A, Plurafac B-25-5®, and an oil having a Specific Gravity between 0.90 and 0.95.

Another embodiment comprises cyclosporin A, Plurafac B-26®, and an oil having a Specific Gravity between 0.90 and 0.95.

Another embodiment comprises cyclosporin A, Plurafac D-25®, and an oil having a Specific Gravity between 0.90 and 0.95.

Another embodiment comprises cyclosporin A, Plurafac LF 131®, and an oil having a Specific Gravity between 0.90 and 0.95.

Another embodiment comprises cyclosporin A, Plurafac LF 4030®, and an oil having a Specific Gravity between 0.90 and 0.95.

Another embodiment comprises cyclosporin A, Plurafac LF 7000®, and an oil having a Specific Gravity between 0.90 and 0.95.

Another embodiment comprises cyclosporin A, Plurafac RA-20®, and an oil having a Specific Gravity between 0.90 and 0.95.

Another embodiment comprises cyclosporin A, Plurafac RA-30®, and an oil having a Specific Gravity between 0.90 and 0.95.

Another embodiment comprises cyclosporin A, Plurafac RA-40®, and an oil having a Specific Gravity between 0.90 and 0.95.

Another embodiment comprises cyclosporin A, Pluronic 10R5®, and an oil having a Specific Gravity between 0.90 and 0.95.

Another embodiment comprises cyclosporin A, Pluronic 17R2®, and an oil having a Specific Gravity between 0.90 and 0.95.

Another embodiment comprises cyclosporin A, Pluronic 17R4®, and an oil having a Specific Gravity between 0.90 and 0.95.

Another embodiment comprises cyclosporin A, Pluronic 25R2®, and an oil having a Specific Gravity between 0.90 and 0.95.

Another embodiment comprises cyclosporin A, Pluronic 25R4®, and an oil having a Specific Gravity between 0.90 and 0.95.

Another embodiment comprises cyclosporin A, Pluronic 31R1®, and an oil having a Specific Gravity between 0.90 and 0.95.

Another embodiment comprises cyclosporin A, Pluronic F108®, and an oil having a Specific Gravity between 0.90 and 0.95.

Another embodiment comprises cyclosporin A, Pluronic F127®, and an oil having a Specific Gravity between 0.90 and 0.95.

Another embodiment comprises cyclosporin A, Pluronic F38®, and an oil having a Specific Gravity between 0.90 and 0.95.

Another embodiment comprises cyclosporin A, Pluronic F68®, and an oil having a Specific Gravity between 0.90 and 0.95.

Another embodiment comprises cyclosporin A, Pluronic F68LF®, and an oil having a Specific Gravity between 0.90 and 0.95.

Another embodiment comprises cyclosporin A, Pluronic F77®, and an oil having a Specific Gravity between 0.90 and 0.95.

Another embodiment comprises cyclosporin A, Pluronic F87®, and an oil having a Specific Gravity between 0.90 and 0.95.

Another embodiment comprises cyclosporin A, Pluronic F88®, and an oil having a Specific Gravity between 0.90 and 0.95.

Another embodiment comprises cyclosporin A, Pluronic F98®, and an oil having a Specific Gravity between 0.90 and 0.95.

Another embodiment comprises cyclosporin A, Pluronic L10®, and an oil having a Specific Gravity between 0.90 and 0.95.

Another embodiment comprises cyclosporin A, Pluronic L101®, and an oil having a Specific Gravity between 0.90 and 0.95.

Another embodiment comprises cyclosporin A, Pluronic L121®, and an oil having a Specific Gravity between 0.90 and 0.95.

Another embodiment comprises cyclosporin A, Pluronic L31®, and an oil having a Specific Gravity between 0.90 and 0.95.

Another embodiment comprises cyclosporin A, Pluronic L35®, and an oil having a Specific Gravity between 0.90 and 0.95.

Another embodiment comprises cyclosporin A, Pluronic L43®, and an oil having a Specific Gravity between 0.90 and 0.95.

Another embodiment comprises cyclosporin A, Pluronic L44®, and an oil having a Specific Gravity between 0.90 and 0.95.

Another embodiment comprises cyclosporin A, Pluronic L61®, and an oil having a Specific Gravity between 0.90 and 0.95.

Another embodiment comprises cyclosporin A, Pluronic L62®, and an oil having a Specific Gravity between 0.90 and 0.95.

Another embodiment comprises cyclosporin A, Pluronic L62D®, and an oil having a Specific Gravity between 0.90 and 0.95.

Another embodiment comprises cyclosporin A, Pluronic L62LF®, and an oil having a Specific Gravity between 0.90 and 0.95.

Another embodiment comprises cyclosporin A, Pluronic L64®, and an oil having a Specific Gravity between 0.90 and 0.95.

Another embodiment comprises cyclosporin A, Pluronic L81®, and an oil having a Specific Gravity between 0.90 and 0.95.

Another embodiment comprises cyclosporin A, Pluronic L92®, and an oil having a Specific Gravity between 0.90 and 0.95.

Another embodiment comprises cyclosporin A, Pluronic P103®, and an oil having a Specific Gravity between 0.90 and 0.95.

Another embodiment comprises cyclosporin A, Pluronic P104®, and an oil having a Specific Gravity between 0.90 and 0.95.

Another embodiment comprises cyclosporin A, Pluronic P105®, and an oil having a Specific Gravity between 0.90 and 0.95.

Another embodiment comprises cyclosporin A, Pluronic P123®, and an oil having a Specific Gravity between 0.90 and 0.95.

Another embodiment comprises cyclosporin A, Pluronic P65®, and an oil having a Specific Gravity between 0.90 and 0.95.

Another embodiment comprises cyclosporin A, Pluronic P84®, and an oil having a Specific Gravity between 0.90 and 0.95.

Another embodiment comprises cyclosporin A, Pluronic P85®, and an oil having a Specific Gravity between 0.90 and 0.95.

Another embodiment comprises cyclosporin A, Polyaldo 10-2-P®, and an oil having a Specific Gravity between 0.90 and 0.95.

Another embodiment comprises cyclosporin A, Polyaldo DGDO®, and an oil having a Specific Gravity between 0.90 and 0.95.

Another embodiment comprises cyclosporin A, Polyaldo HGDS®, and an oil having a Specific Gravity between 0.90 and 0.95.

Another embodiment comprises cyclosporin A, Polyaldo TGMS®, and an oil having a Specific Gravity between 0.90 and 0.95.

Another embodiment comprises cyclosporin A, Protegin V®, and an oil having a Specific Gravity between 0.90 and 0.95.

Another embodiment comprises cyclosporin A, Protegin W®, and an oil having a Specific Gravity between 0.90 and 0.95.

Another embodiment comprises cyclosporin A, Protegin WX®, and an oil having a Specific Gravity between 0.90 and 0.95.

Another embodiment comprises cyclosporin A, Protegin XV®, and an oil having a Specific Gravity between 0.90 and 0.95.

Another embodiment comprises cyclosporin A, Rhodasurf BC-420®, and an oil having a Specific Gravity between 0.90 and 0.95.

Another embodiment comprises cyclosporin A, Rhodasurf BC-610®, and an oil having a Specific Gravity between 0.90 and 0.95.

Another embodiment comprises cyclosporin A, Rhodasurf BC-630®, and an oil having a Specific Gravity between 0.90 and 0.95.

Another embodiment comprises cyclosporin A, Rhodasurf BC-720®, and an oil having a Specific Gravity between 0.90 and 0.95.

Another embodiment comprises cyclosporin A, Rhodasurf BC-840®, and an oil having a Specific Gravity between 0.90 and 0.95.

Another embodiment comprises cyclosporin A, Rhodasun L 1®, and an oil having a Specific Gravity between 0.90 and 0.95.

Another embodiment comprises cyclosporin A, Rhodasurf L 12®, and an oil having a Specific Gravity between 0.90 and 0.95.

Another embodiment comprises cyclosporin A, Rhodasurf L 2®, and an oil having a Specific Gravity between 0.90 and 0.95.

Another embodiment comprises cyclosporin A, Rhodasurf L 20®, and an oil having a Specific Gravity between 0.90 and 0.95.

Another embodiment comprises cyclosporin A, Rhodasurf L 3®, and an oil having a Specific Gravity between 0.90 and 0.95.

Another embodiment comprises cyclosporin A, Rhodasunf L 30®, and an oil having a Specific Gravity between 0.90 and 0.95.

Another embodiment comprises cyclosporin A, Rhodasurf L 4®, and an oil having a Specific Gravity between 0.90 and 0.95.

Another embodiment comprises cyclosporin A, Rhodasurf L 50®, and an oil having a Specific Gravity between 0.90 and 0.95.

Another embodiment comprises cyclosporin A, Rhodasurf L 7/90®, and an oil having a Specific Gravity between 0.90 and 0.95.

Another embodiment comprises cyclosporin A, Rhodasurf LA-3®, and an oil having a Specific Gravity between 0.90 and 0.95.

Another embodiment comprises cyclosporin A, Rhodasurf LA-7®, and an oil having a Specific Gravity between 0.90 and 0.95.

Another embodiment comprises cyclosporin A, Rhodasurf LAN-23®, and an oil having a Specific Gravity between 0.90 and 0.95.

Another embodiment comprises cyclosporin A, Rhodasurf ON-870®, and an oil having a Specific Gravity between 0.90 and 0.95.

Another embodiment comprises cyclosporin A, Rhodasurf ON-877®, and an oil having a Specific Gravity between 0.90 and 0.95.

Another embodiment comprises cyclosporin A, Rhodasurf TDA-8/5®, and an oil having a Specific Gravity between 0.90 and 0.95.

Another embodiment comprises cyclosporin A, Ritapro 100®, and an oil having a Specific Gravity between 0.90 and 0.95.

Another embodiment comprises cyclosporin A, Ritapro 165®, and an oil having a Specific Gravity between 0.90 and 0.95.

Another embodiment comprises cyclosporin A, Ritapro 200®, and an oil having a Specific Gravity between 0.90 and 0.95.

Another embodiment comprises cyclosporin A, Ritapro 300®, and an oil having a Specific Gravity between 0.90 and 0.95.

Another embodiment comprises cyclosporin A, Ritoleth 10®, and an oil having a Specific Gravity between 0.90 and 0.95.

Another embodiment comprises cyclosporin A, Ritoleth 2®, and an oil having a Specific Gravity between 0.90 and 0.95.

Another embodiment comprises cyclosporin A, Ritoleth 20®, and an oil having a Specific Gravity between 0.90 and 0.95.

Another embodiment comprises cyclosporin A, Ritoleth 5®, and an oil having a Specific Gravity between 0.90 and 0.95.

Another embodiment comprises cyclosporin A, Santone 10-10-0®, and an oil having a Specific Gravity between 0.90 and 0.95.

Another embodiment comprises cyclosporin A, Santone 3-1-S XTR®, and an oil having a Specific Gravity between 0.90 and 0.95.

Another embodiment comprises cyclosporin A, Santone 3-1-SH®, and an oil having a Specific Gravity between 0.90 and 0.95.

Another embodiment comprises cyclosporin A, Santone 8-1-O®, and an oil having a Specific Gravity between 0.90 and 0.95.

Another embodiment comprises cyclosporin A, Solulan 16®, and an oil having a Specific Gravity between 0.90 and 0.95.

Another embodiment comprises cyclosporin A, Solulan 75®, and an oil having a Specific Gravity between 0.90 and 0.95.

Another embodiment comprises cyclosporin A, Solulan C-24®, and an oil having a Specific Gravity between 0.90 and 0.95.

Another embodiment comprises cyclosporin A, Solulan L-575®, and an oil having a Specific Gravity between 0.90 and 0.95.

Another embodiment comprises cyclosporin A, Standamox CAW®, and an oil having a Specific Gravity between 0.90 and 0.95.

Another embodiment comprises cyclosporin A, Surfine AZI-A®, and an oil having a Specific Gravity between 0.90 and 0.95.

Another embodiment comprises cyclosporin A, Surfonic CO-15®, and an oil having a Specific Gravity between 0.90 and 0.95.

Another embodiment comprises cyclosporin A, Surfonic CO-25®, and an oil having a Specific Gravity between 0.90 and 0.95.

Another embodiment comprises cyclosporin A, Surfonic CO-30®, and an oil having a Specific Gravity between 0.90 and 0.95.

Another embodiment comprises cyclosporin A, Surfonic CO-36®, and an oil having a Specific Gravity between 0.90 and 0.95.

Another embodiment comprises cyclosporin A, Surfonic CO-42®, and an oil having a Specific Gravity between 0.90 and 0.95.

Another embodiment comprises cyclosporin A, Surfonic E400-MO®, and an oil having a Specific Gravity between 0.90 and 0.95.

Another embodiment comprises cyclosporin A, Surfonic L12-3®, and an oil having a Specific Gravity between 0.90 and 0.95.

Another embodiment comprises cyclosporin A, Surfonic L12-6®, and an oil having a Specific Gravity between 0.90 and 0.95.

Another embodiment comprises cyclosporin A, Surfonic L12-8®, and an oil having a Specific Gravity between 0.90 and 0.95.

Another embodiment comprises cyclosporin A, Surfonic L24-12®, and an oil having a Specific Gravity between 0.90 and 0.95.

Another embodiment comprises cyclosporin A, Surfonic L24-2®, and an oil having a Specific Gravity between 0.90 and 0.95.

Another embodiment comprises cyclosporin A, Surfonic L24-22®, and an oil having a Specific Gravity between 0.90 and 0.95.

Another embodiment comprises cyclosporin A, Surfonic L24-3®, and an oil having a Specific Gravity between 0.90 and 0.95.

Another embodiment comprises cyclosporin A, Surfonic L24-4®, and an oil having a Specific Gravity between 0.90 and 0.95.

Another embodiment comprises cyclosporin A, Surfonic L24-5®, and an oil having a Specific Gravity between 0.90 and 0.95.

Another embodiment comprises cyclosporin A, Surfonic L24-7®, and an oil having a Specific Gravity between 0.90 and 0.95.

Another embodiment comprises cyclosporin A, Surfonic L24-7.1®, and an oil having a Specific Gravity between 0.90 and 0.95.

Another embodiment comprises cyclosporin A, Surfonic L24-9®, and an oil having a Specific Gravity between 0.90 and 0.95.

Another embodiment comprises cyclosporin A, Surfonic LF-17®, and an oil having a Specific Gravity between 0.90 and 0.95.

Another embodiment comprises cyclosporin A, Surfonic LF-18®, and an oil having a Specific Gravity between 0.90 and 0.95.

Another embodiment comprises cyclosporin A, Surfonic LF-37®, and an oil having a Specific Gravity between 0.90 and 0.95.

Another embodiment comprises cyclosporin A, Surfonic LF-41®, and an oil having a Specific Gravity between 0.90 and 0.95.

Another embodiment comprises cyclosporin A, Surfonic LF-42®, and an oil having a Specific Gravity between 0.90 and 0.95.

Another embodiment comprises cyclosporin A, Surfonic LF-47®, and an oil having a Specific Gravity between 0.90 and 0.95.

Another embodiment comprises cyclosporin A, Surfonic LF-68®, and an oil having a Specific Gravity between 0.90 and 0.95.

Another embodiment comprises cyclosporin A, Surfonic N-10®, and an oil having a Specific Gravity between 0.90 and 0.95.

Another embodiment comprises cyclosporin A, Surfonic N-100®, and an oil having a Specific Gravity between 0.90 and 0.95.

Another embodiment comprises cyclosporin A, Surfonic N-102®, and an oil having a Specific Gravity between 0.90 and 0.95.

Another embodiment comprises cyclosporin A, Surfonic N-120®, and an oil having a Specific Gravity between 0.90 and 0.95.

Another embodiment comprises cyclosporin A, Surfonic N-150®, and an oil having a Specific Gravity between 0.90 and 0.95.

Another embodiment comprises cyclosporin A, Surfonic N-200®, and an oil having a Specific Gravity between 0.90 and 0.95.

Another embodiment comprises cyclosporin A, Surfonic N-300®, and an oil having a Specific Gravity between 0.90 and 0.95.

Another embodiment comprises cyclosporin A, Surfonic N-31.5®, and an oil having a Specific Gravity between 0.90 and 0.95.

Another embodiment comprises cyclosporin A, Surfonic N-40®, and an oil having a Specific Gravity between 0.90 and 0.95.

Another embodiment comprises cyclosporin A, Sunfonic N-400®, and an oil having a Specific Gravity between 0.90 and 0.95.

Another embodiment comprises cyclosporin A, Surfonic N-60®, and an oil having a Specific Gravity between 0.90 and 0.95.

Another embodiment comprises cyclosporin A, Surfonic N-85®, and an oil having a Specific Gravity between 0.90 and 0.95.

Another embodiment comprises cyclosporin A, Sunfonic N-95®, and an oil having a Specific Gravity between 0.90 and 0.95.

Another embodiment comprises cyclosporin A, Surfonic NB-18®, and an oil having a Specific Gravity between 0.90 and 0.95.

Another embodiment comprises cyclosporin A, Surfonic NB-23®, and an oil having a Specific Gravity between 0.90 and 0.95.

Another embodiment comprises cyclosporin A, Surfonic NB-25®, and an oil having a Specific Gravity between 0.90 and 0.95.

Another embodiment comprises cyclosporin A, Surfonic NB-307®, and an oil having a Specific Gravity between 0.90 and 0.95.

Another embodiment comprises cyclosporin A, Surfonic NB-31®, and an oil having a Specific Gravity between 0.90 and 0.95.

Another embodiment comprises cyclosporin A, Surfonic NB-41®, and an oil having a Specific Gravity between 0.90 and 0.95.

Another embodiment comprises cyclosporin A, Surfonic NB-43®, and an oil having a Specific Gravity between 0.90 and 0.95.

Another embodiment comprises cyclosporin A, Surfonic OP-100®, and an oil having a Specific Gravity between 0.90 and 0.95.

Another embodiment comprises cyclosporin A, Surfonic OP-120®, and an oil having a Specific Gravity between 0.90 and 0.95.

Another embodiment comprises cyclosporin A, Surfonic OP-15®, and an oil having a Specific Gravity between 0.90 and 0.95.

Another embodiment comprises cyclosporin A, Surfonic OP-35®, and an oil having a Specific Gravity between 0.90 and 0.95.

Another embodiment comprises cyclosporin A, Surfonic OP-70®, and an oil having a Specific Gravity between 0.90 and 0.95.

Another embodiment comprises cyclosporin A, Surfonic P1®, and an oil having a Specific Gravity between 0.90 and 0.95.

Another embodiment comprises cyclosporin A, Surfonic P3®, and an oil having a Specific Gravity between 0.90 and 0.95.

Another embodiment comprises cyclosporin A, Surfonic P5®, and an oil having a Specific Gravity between 0.90 and 0.95.

Another embodiment comprises cyclosporin A, Surfonic P6®, and an oil having a Specific Gravity between 0.90 and 0.95.

Another embodiment comprises cyclosporin A, Surfonic POA-17R2®, and an oil having a Specific Gravity between 0.90 and 0.95.

Another embodiment comprises cyclosporin A, Surfonic POA-25R2®, and an oil having a Specific Gravity between 0.90 and 0.95.

Another embodiment comprises cyclosporin A, Surfonic TDA-11®, and an oil having a Specific Gravity between 0.90 and 0.95.

Another embodiment comprises cyclosporin A, Surfonic TDA-3B®, and an oil having a Specific Gravity between 0.90 and 0.95.

Another embodiment comprises cyclosporin A, Surfonic TDA-6®, and an oil having a Specific Gravity between 0.90 and 0.95.

Another embodiment comprises cyclosporin A, Surfonic TDA-6/88®, and an oil having a Specific Gravity between 0.90 and 0.95.

Another embodiment comprises cyclosporin A, Surfonic TDA-8®, and an oil having a Specific Gravity between 0.90 and 0.95.

Another embodiment comprises cyclosporin A, Surfonic TDA-8/90®, and an oil having a Specific Gravity between 0.90 and 0.95.

Another embodiment comprises cyclosporin A, Surfonic TDA-9®, and an oil having a Specific Gravity between 0.90 and 0.95.

Another embodiment comprises cyclosporin A, Surfonic TX-CCN®, and an oil having a Specific Gravity between 0.90 and 0.95.

Another embodiment comprises cyclosporin A, Tagat CH 40®, and an oil having a Specific Gravity between 0.90 and 0.95.

Another embodiment comprises cyclosporin A, Tagat CH 60®, and an oil having a Specific Gravity between 0.90 and 0.95.

Another embodiment comprises cyclosporin A, Tagat L 2®, and an oil having a Specific Gravity between 0.90 and 0.95.

Another embodiment comprises cyclosporin A, Tagat S®, and an oil having a Specific Gravity between 0.90 and 0.95.

Another embodiment comprises cyclosporin A, Tagat S 2®, and an oil having a Specific Gravity between 0.90 and 0.95.

Another embodiment comprises cyclosporin A, Tally 100 Plus®, and an oil having a Specific Gravity between 0.90 and 0.95.

Another embodiment comprises cyclosporin A, T-Det A106®, and an oil having a Specific Gravity between 0.90 and 0.95.

Another embodiment comprises cyclosporin A, T-Det A109®, and an oil having a Specific Gravity between 0.90 and 0.95.

Another embodiment comprises cyclosporin A, T-Det A136®, and an oil having a Specific Gravity between 0.90 and 0.95.

Another embodiment comprises cyclosporin A, T-Det A139®, and an oil having a Specific Gravity between 0.90 and 0.95.

Another embodiment comprises cyclosporin A, T-Det A2412®, and an oil having a Specific Gravity between 0.90 and 0.95.

Another embodiment comprises cyclosporin A, T-Det A243®, and an oil having a Specific Gravity between 0.90 and 0.95.

Another embodiment comprises cyclosporin A, T-Det A247®, and an oil having a Specific Gravity between 0.90 and 0.95.

Another embodiment comprises cyclosporin A, T-Det A249®, and an oil having a Specific Gravity between 0.90 and 0.95.

Another embodiment comprises cyclosporin A, T-Det A467®, and an oil having a Specific Gravity between 0.90 and 0.95.

Another embodiment comprises cyclosporin A, T-Det COE®, and an oil having a Specific Gravity between 0.90 and 0.95.

Another embodiment comprises cyclosporin A, T-Det DD-10®, and an oil having a Specific Gravity between 0.90 and 0.95.

Another embodiment comprises cyclosporin A, T-Det DD-5®, and an oil having a Specific Gravity between 0.90 and 0.95.

Another embodiment comprises cyclosporin A, T-Det DD-7®, and an oil having a Specific Gravity between 0.90 and 0.95.

Another embodiment comprises cyclosporin A, T-Det EPO-104®, and an oil having a Specific Gravity between 0.90 and 0.95.

Another embodiment comprises cyclosporin A, T-Det EPO-61®, and an oil having a Specific Gravity between 0.90 and 0.95.

Another embodiment comprises cyclosporin A, T-Det EPO-62®, and an oil having a Specific Gravity between 0.90 and 0.95.

Another embodiment comprises cyclosporin A, T-Det EPO-64®, and an oil having a Specific Gravity between 0.90 and 0.95.

Another embodiment comprises cyclosporin A, T-Det N-1.5®, and an oil having a Specific Gravity between 0.90 and 0.95.

Another embodiment comprises cyclosporin A, T-Det N-10.5®, and an oil having a Specific Gravity between 0.90 and 0.95.

Another embodiment comprises cyclosporin A, T-Det N-100®, and an oil having a Specific Gravity between 0.90 and 0.95.

Another embodiment comprises cyclosporin A, T-Det N-1007®, and an oil having a Specific Gravity between 0.90 and 0.95.

Another embodiment comprises cyclosporin A, T-Det N-12®, and an oil having a Specific Gravity between 0.90 and 0.95.

Another embodiment comprises cyclosporin A, T-Det N-14®, and an oil having a Specific Gravity between 0.90 and 0.95.

Another embodiment comprises cyclosporin A, T-Det N-20®, and an oil having a Specific Gravity between 0.90 and 0.95.

Another embodiment comprises cyclosporin A, T-Det N-30®, and an oil having a Specific Gravity between 0.90 and 0.95.

Another embodiment comprises cyclosporin A, T-Det N-307®, and an oil having a Specific Gravity between 0.90 and 0.95.

Another embodiment comprises cyclosporin A, T-Det N-4®, and an oil having a Specific Gravity between 0.90 and 0.95.

Another embodiment comprises cyclosporin A, T-Det N-40®, and an oil having a Specific Gravity between 0.90 and 0.95.

Another embodiment comprises cyclosporin A, T-Det N-407®, and an oil having a Specific Gravity between 0.90 and 0.95.

Another embodiment comprises cyclosporin A, T-Det N-50®, and an oil having a Specific Gravity between 0.90 and 0.95.

Another embodiment comprises cyclosporin A, T-Det N-507®, and an oil having a Specific Gravity between 0.90 and 0.95.

Another embodiment comprises cyclosporin A, T-Det N-6®, and an oil having a Specific Gravity between 0.90 and 0.95.

Another embodiment comprises cyclosporin A, T-Det N-70®, and an oil having a Specific Gravity between 0.90 and 0.95.

Another embodiment comprises cyclosporin A, T-Det N-8®, and an oil having a Specific Gravity between 0.90 and 0.95.

Another embodiment comprises cyclosporin A, T-Det N-9.5®, and an oil having a Specific Gravity between 0.90 and 0.95.

Another embodiment comprises cyclosporin A, T-Det O-12®, and an oil having a Specific Gravity between 0.90 and 0.95.

Another embodiment comprises cyclosporin A, T-Det O-165®, and an oil having a Specific Gravity between 0.90 and 0.95.

Another embodiment comprises cyclosporin A, T-Det O-307®, and an oil having a Specific Gravity between 0.90 and 0.95.

Another embodiment comprises cyclosporin A, T-Det O-40®, and an oil having a Specific Gravity between 0.90 and 0.95.

Another embodiment comprises cyclosporin A, T-Det O-407®, and an oil having a Specific Gravity between 0.90 and 0.95.

Another embodiment comprises cyclosporin A, T-Det O-6®, and an oil having a Specific Gravity between 0.90 and 0.95.

Another embodiment comprises cyclosporin A, T-Det O-8®, and an oil having a Specific Gravity between 0.90 and 0.95.

Another embodiment comprises cyclosporin A, T-Det O-9®, and an oil having a Specific Gravity between 0.90 and 0.95.

Another embodiment comprises cyclosporin A, Tegin®, and an oil having a Specific Gravity between 0.90 and 0.95.

Another embodiment comprises cyclosporin A, Tergitol 15-S-12®, and an oil having a Specific Gravity between 0.90 and 0.95.

Another embodiment comprises cyclosporin A, Tergitol 15-S-15®, and an oil having a Specific Gravity between 0.90 and 0.95.

Another embodiment comprises cyclosporin A, Tergitol 15-S-20®, and an oil having a Specific Gravity between 0.90 and 0.95.

Another embodiment comprises cyclosporin A, Tergitol 15-S-20 (80AQ)®, and an oil having a Specific Gravity between 0.90 and 0.95.

Another embodiment comprises cyclosporin A, Tergitol 15-S-3®, and an oil having a Specific Gravity between 0.90 and 0.95.

Another embodiment comprises cyclosporin A, Tergitol 15-S-30®, and an oil having a Specific Gravity between 0.90 and 0.95.

Another embodiment comprises cyclosporin A, Tergitol 15-S-40®, and an oil having a Specific Gravity between 0.90 and 0.95.

Another embodiment comprises cyclosporin A, Tergitol 15-S-40 (70AQ)®, and an oil having a Specific Gravity between 0.90 and 0.95.

Another embodiment comprises cyclosporin A, Tergitol 15-S-5®, and an oil having a Specific Gravity between 0.90 and 0.95.

Another embodiment comprises cyclosporin A, Tergitol 15-S-7®, and an oil having a Specific Gravity between 0.90 and 0.95.

Another embodiment comprises cyclosporin A, Tergitol 15-S-9®, and an oil having a Specific Gravity between 0.90 and 0.95.

Another embodiment comprises cyclosporin A, Tergitol L-101®, and an oil having a Specific Gravity between 0.90 and 0.95.

Another embodiment comprises cyclosporin A, Tergitol L-61®, and an oil having a Specific Gravity between 0.90 and 0.95.

Another embodiment comprises cyclosporin A, Tergitol L-62®, and an oil having a Specific Gravity between 0.90 and 0.95.

Another embodiment comprises cyclosporin A, Tergitol L-64®, and an oil having a Specific Gravity between 0.90 and 0.95.

Another embodiment comprises cyclosporin A, Tergitol L-81®, and an oil having a Specific Gravity between 0.90 and 0.95.

Another embodiment comprises cyclosporin A, Tergitol NP-10®, and an oil having a Specific Gravity between 0.90 and 0.95.

Another embodiment comprises cyclosporin A, Tergitol NP-11®, and an oil having a Specific Gravity between 0.90 and 0.95.

Another embodiment comprises cyclosporin A, Tergitol NP-12®, and an oil having a Specific Gravity between 0.90 and 0.95.

Another embodiment comprises cyclosporin A, Tergitol NP-13®, and an oil having a Specific Gravity between 0.90 and 0.95.

Another embodiment comprises cyclosporin A, Tergitol NP-15®, and an oil having a Specific Gravity between 0.90 and 0.95.

Another embodiment comprises cyclosporin A, Tergitol NP-30®, and an oil having a Specific Gravity between 0.90 and 0.95.

Another embodiment comprises cyclosporin A, Tergitol NP-30 (70 AQ)®, and an oil having a Specific Gravity between 0.90 and 0.95.

Another embodiment comprises cyclosporin A, Tergitol NP-4®, and an oil having a Specific Gravity between 0.90 and 0.95.

Another embodiment comprises cyclosporin A, Tergitol NP-40®, and an oil having a Specific Gravity between 0.90 and 0.95.

Another embodiment comprises cyclosporin A, Tergitol NP-40 (70 AQ)®, and an oil having a Specific Gravity between 0.90 and 0.95.

Another embodiment comprises cyclosporin A, Tergitol NP-50 (70 AQ)®, and an oil having a Specific Gravity between 0.90 and 0.95.

Another embodiment comprises cyclosporin A, Tergitol NP-55 (70 AQ)®, and an oil having a Specific Gravity between 0.90 and 0.95.

Another embodiment comprises cyclosporin A, Tergitol NP-6®, and an oil having a Specific Gravity between 0.90 and 0.95.

Another embodiment comprises cyclosporin A, Tergitol NP-7®, and an oil having a Specific Gravity between 0.90 and 0.95.

Another embodiment comprises cyclosporin A, Tergitol NP-70 (70 AQ)®, and an oil having a Specific Gravity between 0.90 and 0.95.

Another embodiment comprises cyclosporin A, Tergitol NP-8®, and an oil having a Specific Gravity between 0.90 and 0.95.

Another embodiment comprises cyclosporin A, Tergitol NP-9®, and an oil having a Specific Gravity between 0.90 and 0.95.

Another embodiment comprises cyclosporin A, Tergitol NP-9.5®, and an oil having a Specific Gravity between 0.90 and 0.95.

Another embodiment comprises cyclosporin A, Tergitol TMN-10®, and an oil having a Specific Gravity between 0.90 and 0.95.

Another embodiment comprises cyclosporin A, Tergitol TMN-3®, and an oil having a Specific Gravity between 0.90 and 0.95.

Another embodiment comprises cyclosporin A, Tergitol TMN-6®, and an oil having a Specific Gravity between 0.90 and 0.95.

Another embodiment comprises cyclosporin A, Tergitol XD®, and an oil having a Specific Gravity between 0.90 and 0.95.

Another embodiment comprises cyclosporin A, Tergitol XH®, and an oil having a Specific Gravity between 0.90 and 0.95.

Another embodiment comprises cyclosporin A, Tergitol XJ®, and an oil having a Specific Gravity between 0.90 and 0.95.

Another embodiment comprises cyclosporin A, Tetronic 1107®, and an oil having a Specific Gravity between 0.90 and 0.95.

Another embodiment comprises cyclosporin A, Tetronic 1307®, and an oil having a Specific Gravity between 0.90 and 0.95.

Another embodiment comprises cyclosporin A, Tetronic 150R1®, and an oil having a Specific Gravity between 0.90 and 0.95.

Another embodiment comprises cyclosporin A, Tetronic 304®, and an oil having a Specific Gravity between 0.90 and 0.95.

Another embodiment comprises cyclosporin A, Tetronic 701®, and an oil having a Specific Gravity between 0.90 and 0.95.

Another embodiment comprises cyclosporin A, Tetronic 901®, and an oil having a Specific Gravity between 0.90 and 0.95.

Another embodiment comprises cyclosporin A, Tetronic 904®, and an oil having a Specific Gravity between 0.90 and 0.95.

Another embodiment comprises cyclosporin A, Tetronic 908®, and an oil having a Specific Gravity between 0.90 and 0.95.

Another embodiment comprises cyclosporin A, Tetronic 90R4®, and an oil having a Specific Gravity between 0.90 and 0.95.

Another embodiment comprises cyclosporin A, Tomadol 1-3®, and an oil having a Specific Gravity between 0.90 and 0.95.

Another embodiment comprises cyclosporin A, Tomadol 1-5®, and an oil having a Specific Gravity between 0.90 and 0.95.

Another embodiment comprises cyclosporin A, Tomadol 1-7®, and an oil having a Specific Gravity between 0.90 and 0.95.

Another embodiment comprises cyclosporin A, Tomadol 23-1®, and an oil having a Specific Gravity between 0.90 and 0.95.

Another embodiment comprises cyclosporin A, Tomadol 23-3®, and an oil having a Specific Gravity between 0.90 and 0.95.

Another embodiment comprises cyclosporin A, Tomadol 23-5®, and an oil having a Specific Gravity between 0.90 and 0.95.

Another embodiment comprises cyclosporin A, Tomadol 23-6.5®, and an oil having a Specific Gravity between 0.90 and 0.95.

Another embodiment comprises cyclosporin A, Tomadol 25-12®, and an oil having a Specific Gravity between 0.90 and 0.95.

Another embodiment comprises cyclosporin A, Tomadol 25-3®, and an oil having a Specific Gravity between 0.90 and 0.95.

Another embodiment comprises cyclosporin A, Tomadol 25-7®, and an oil having a Specific Gravity between 0.90 and 0.95.

Another embodiment comprises cyclosporin A, Tomadol 25-9®, and an oil having a Specific Gravity between 0.90 and 0.95.

Another embodiment comprises cyclosporin A, Tomadol 45-13®, and an oil having a Specific Gravity between 0.90 and 0.95.

Another embodiment comprises cyclosporin A, Tomadol 45-2.5®, and an oil having a Specific Gravity between 0.90 and 0.95.

Another embodiment comprises cyclosporin A, Tomadol 45-7®, and an oil having a Specific Gravity between 0.90 and 0.95.

Another embodiment comprises cyclosporin A, Tomadol 91-2.5®, and an oil having a Specific Gravity between 0.90 and 0.95.

Another embodiment comprises cyclosporin A, Tomadol 91-6®, and an oil having a Specific Gravity between 0.90 and 0.95.

Another embodiment comprises cyclosporin A, Tomadol 91-8®, and an oil having a Specific Gravity between 0.90 and 0.95.

Another embodiment comprises cyclosporin A, Triton X-100®, and an oil having a Specific Gravity between 0.90 and 0.95.

Another embodiment comprises cyclosporin A, Triton X-102®, and an oil having a Specific Gravity between 0.90 and 0.95.

Another embodiment comprises cyclosporin A, Triton X-114®, and an oil having a Specific Gravity between 0.90 and 0.95.

Another embodiment comprises cyclosporin A, Triton X-15®, and an oil having a Specific Gravity between 0.90 and 0.95.

Another embodiment comprises cyclosporin A, Triton X-165 (70%)®, and an oil having a Specific Gravity between 0.90 and 0.95.

Another embodiment comprises cyclosporin A, Triton X-207®, and an oil having a Specific Gravity between 0.90 and 0.95.

Another embodiment comprises cyclosporin A, Triton X-305 (70%)®, and an oil having a Specific Gravity between 0.90 and 0.95.

Another embodiment comprises cyclosporin A, Triton X-35®, and an oil having a Specific Gravity between 0.90 and 0.95.

Another embodiment comprises cyclosporin A, Triton X-405 (70%)®, and an oil having a Specific Gravity between 0.90 and 0.95.

Another embodiment comprises cyclosporin A, Triton X-45®, and an oil having a Specific Gravity between 0.90 and 0.95.

Another embodiment comprises cyclosporin A, Triton X-705 (70%)®, and an oil having a Specific Gravity between 0.90 and 0.95.

Another embodiment comprises cyclosporin A, Volpo S-10®, and an oil having a Specific Gravity between 0.90 and 0.95.

Another embodiment comprises cyclosporin A, Volpo S-2®, and an oil having a Specific Gravity between 0.90 and 0.95.

Another embodiment comprises cyclosporin A, Volpo S-20®, and an oil having a Specific Gravity between 0.90 and 0.95.

Another embodiment comprises cyclosporin A, Volpo-10®, and an oil having a Specific Gravity between 0.90 and 0.95.

Another embodiment comprises cyclosporin A, Volpo-20®, and an oil having a Specific Gravity between 0.90 and 0.95.

Another embodiment comprises cyclosporin A, Volpo-3®, and an oil having a Specific Gravity between 0.90 and 0.95.

Another embodiment comprises cyclosporin A, Volpo-5®, and an oil having a Specific Gravity between 0.90 and 0.95.

Another embodiment comprises cyclosporin A, Abex 2515®, and Almond oil.

Another embodiment comprises cyclosporin A, Abex 2525/40®, and Almond oil.

Another embodiment comprises cyclosporin A, Abex 2535®, and Almond oil.

Another embodiment comprises cyclosporin A, Abex 2545®, and Almond oil.

Another embodiment comprises cyclosporin A, Acconon E®, and Almond oil.

Another embodiment comprises cyclosporin A, Alfonic 1216CO-9®, and Almond oil.

Another embodiment comprises cyclosporin A, Alfonic 1412-3®, and Almond oil.

Another embodiment comprises cyclosporin A, Alfonic TDA-12®, and Almond oil.

Another embodiment comprises cyclosporin A, Alfonic TDA-3®, and Almond oil.

Another embodiment comprises cyclosporin A, Alfonic TDA-4®, and Almond oil.

Another embodiment comprises cyclosporin A, Alfonic TDA-6®, and Almond oil.

Another embodiment comprises cyclosporin A, Alfonic TDA-7®, and Almond oil.

Another embodiment comprises cyclosporin A, Alfonic TDA-8®, and Almond oil.

Another embodiment comprises cyclosporin A, Alfonic TDA-9®, and Almond oil.

Another embodiment comprises cyclosporin A, Alkamuls 400-DO®, and Almond oil.

Another embodiment comprises cyclosporin A, Alkamuls 400-MO/E®, and Almond oil.

Another embodiment comprises cyclosporin A, Ameroxol OE-10®, and Almond oil.

Another embodiment comprises cyclosporin A, Ameroxol OE-2®, and Almond oil.

Another embodiment comprises cyclosporin A, Ameroxol OE-20®, and Almond oil.

Another embodiment comprises cyclosporin A, Armix 176®, and Almond oil.

Another embodiment comprises cyclosporin A, Armix 180-C®, and Almond oil.

Another embodiment comprises cyclosporin A, Armix 183®, and Almond oil.

Another embodiment comprises cyclosporin A, Brij 35®, and Almond oil.

Another embodiment comprises cyclosporin A, Brij 35 Liquid®, and Almond oil.

Another embodiment comprises cyclosporin A, Brij 35 SP®, and Almond oil.

Another embodiment comprises cyclosporin A, Canasol MJ 2109®, and Almond oil.

Another embodiment comprises cyclosporin A, Canasol MJ 52®, and Almond oil.

Another embodiment comprises cyclosporin A, Canasol R 3600®, and Almond oil.

Another embodiment comprises cyclosporin A, Canasol R 3603®, and Almond oil.

Another embodiment comprises cyclosporin A, Canasol R 4000 H®, and Almond oil.

Another embodiment comprises cyclosporin A, Cedepal CA-210®, and Almond oil.

Another embodiment comprises cyclosporin A, Cedepal CA-520®, and Almond oil.

Another embodiment comprises cyclosporin A, Cedepal CA-630®, and Almond oil.

Another embodiment comprises cyclosporin A, Cedepal CA-720®, and Almond oil.

Another embodiment comprises cyclosporin A, Cedepal CA-890®, and Almond oil.

Another embodiment comprises cyclosporin A, Cedepal CA-897®, and Almond oil.

Another embodiment comprises cyclosporin A, Cedepal CO-210®, and Almond oil.

Another embodiment comprises cyclosporin A, Cedepal CO-430®, and Almond oil.

Another embodiment comprises cyclosporin A, Cedepal CO-530®, and Almond oil.

Another embodiment comprises cyclosporin A, Cedepal CO-630®, and Almond oil.

Another embodiment comprises cyclosporin A, Cedepal CO-710®, and Almond oil.

Another embodiment comprises cyclosporin A, Cedepal CO-730®, and Almond oil.

Another embodiment comprises cyclosporin A, Cedepal CO-880®, and Almond oil.

Another embodiment comprises cyclosporin A, Cedepal CO-887®, and Almond oil.

Another embodiment comprises cyclosporin A, Cedepal CO-890®, and Almond oil.

Another embodiment comprises cyclosporin A, Cedepal CO-897®, and Almond oil.

Another embodiment comprises cyclosporin A, Cedepal CO-970®, and Almond oil.

Another embodiment comprises cyclosporin A, Cedepal CO-977®, and Almond oil.

Another embodiment comprises cyclosporin A, Cedepal CO-990®, and Almond oil.

Another embodiment comprises cyclosporin A, Cedepal CO-997®, and Almond oil.

Another embodiment comprises cyclosporin A, Chemal BP 261®, and Almond oil.

Another embodiment comprises cyclosporin A, Chemal BP 262®, and Almond oil.

Another embodiment comprises cyclosporin A, Chemal BP 3172®, and Almond oil.

Another embodiment comprises cyclosporin A, Chemal BP 3174®, and Almond oil.

Another embodiment comprises cyclosporin A, Chemal DA-4®, and Almond oil.

Another embodiment comprises cyclosporin A, Chemal DA-6®, and Almond oil.

Another embodiment comprises cyclosporin A, Chemal DA-9®, and Almond oil.

Another embodiment comprises cyclosporin A, Chemal LA-23®, and Almond oil.

Another embodiment comprises cyclosporin A, Chemal LA-4®, and Almond oil.

Another embodiment comprises cyclosporin A, Chemal LA-9®, and Almond oil.

Another embodiment comprises cyclosporin A, Chemal OA-20G®, and Almond oil.

Another embodiment comprises cyclosporin A, Chemal OA-5®, and Almond oil.

Another embodiment comprises cyclosporin A, Chemal OA-9®, and Almond oil.

Another embodiment comprises cyclosporin A, Chemal TDA-12®, and Almond oil.

Another embodiment comprises cyclosporin A, Chemal TDA-15®, and Almond oil.

Another embodiment comprises cyclosporin A, Chemal TDA-3®, and Almond oil.

Another embodiment comprises cyclosporin A, Chemal TDA-6®, and Almond oil.

Another embodiment comprises cyclosporin A, Chemal TDA-9®, and Almond oil.

Another embodiment comprises cyclosporin A, Chemax CO-16®, and Almond oil.

Another embodiment comprises cyclosporin A, Chemax CO-200/50®, and Almond oil.

Another embodiment comprises cyclosporin A, Chemax CO-25®, and Almond oil.

Another embodiment comprises cyclosporin A, Chemax CO-30®, and Almond oil.

Another embodiment comprises cyclosporin A, Chemax CO-36®, and Almond oil.

Another embodiment comprises cyclosporin A, Chemax CO-40®, and Almond oil.

Another embodiment comprises cyclosporin A, Chemax CO-5®, and Almond oil.

Another embodiment comprises cyclosporin A, Chemax CO-80®, and Almond oil.

Another embodiment comprises cyclosporin A, Chemax DNP-150/50®, and Almond oil.

Another embodiment comprises cyclosporin A, Chemax DNP-18®, and Almond oil.

Another embodiment comprises cyclosporin A, Chemax DNP-8®, and Almond oil.

Another embodiment comprises cyclosporin A, Chemax E 200 ML®, and Almond oil.

Another embodiment comprises cyclosporin A, Chemax E 200 MO®, and Almond oil.

Another embodiment comprises cyclosporin A, Chemax E 400 ML®, and Almond oil.

Another embodiment comprises cyclosporin A, Chemax E 400 MO®, and Almond oil.

Another embodiment comprises cyclosporin A, Chemax E 600 ML®, and Almond oil.

Another embodiment comprises cyclosporin A, Chemax E 600 MO®, and Almond oil.

Another embodiment comprises cyclosporin A, Chemax E1000 MO®, and Almond oil.

Another embodiment comprises cyclosporin A, Chemax E1000 MS®, and Almond oil.

Another embodiment comprises cyclosporin A, Chemax E200 MS®, and Almond oil.

Another embodiment comprises cyclosporin A, Chemax E400 MS®, and Almond oil.

Another embodiment comprises cyclosporin A, Chemax E600 MS®, and Almond oil.

Another embodiment comprises cyclosporin A, Chemax HCO-16®, and Almond oil.

Another embodiment comprises cyclosporin A, Chemax HCO-200/50®, and Almond oil.

Another embodiment comprises cyclosporin A, Chemax HCO-25®, and Almond oil.

Another embodiment comprises cyclosporin A, Chemax HCO-5®, and Almond oil.

Another embodiment comprises cyclosporin A, Chemax TO-16®, and Almond oil.

Another embodiment comprises cyclosporin A, Chemax TO-8®, and Almond oil.

Another embodiment comprises cyclosporin A, Colalube 3409®, and Almond oil.

Another embodiment comprises cyclosporin A, Colalube 3411®, and Almond oil.

Another embodiment comprises cyclosporin A, Colalube 3413®, and Almond oil.

Another embodiment comprises cyclosporin A, Colalube 3414®, and Almond oil.

Another embodiment comprises cyclosporin A, Colalube 3415®, and Almond oil.

Another embodiment comprises cyclosporin A, Colalube 3416®, and Almond oil.

Another embodiment comprises cyclosporin A, Colalube 3417®, and Almond oil.

Another embodiment comprises cyclosporin A, Delonic NPE-100®, and Almond oil.

Another embodiment comprises cyclosporin A, Delonic NPE-30®, and Almond oil.

Another embodiment comprises cyclosporin A, Delonic NPE-40®, and Almond oil.

Another embodiment comprises cyclosporin A, Delonic OPE-10®, and Almond oil.

Another embodiment comprises cyclosporin A, Delonic OPE-12®, and Almond oil.

Another embodiment comprises cyclosporin A, Delonic OPE-30®, and Almond oil.

Another embodiment comprises cyclosporin A, Delonic OPE-40®, and Almond oil.

Another embodiment comprises cyclosporin A, Delonic OPE-5®, and Almond oil.

Another embodiment comprises cyclosporin A, Delonic OPE-7.5®, and Almond oil.

Another embodiment comprises cyclosporin A, DePeg 16-CO®, and Almond oil.

Another embodiment comprises cyclosporin A, DePeg 25-CO®, and Almond oil.

Another embodiment comprises cyclosporin A, DePeg 30-CO®, and Almond oil.

Another embodiment comprises cyclosporin A, DePeg 40-CO®, and Almond oil.

Another embodiment comprises cyclosporin A, DePeg 5-CO®, and Almond oil.

Another embodiment comprises cyclosporin A, DePeg 80-CO®, and Almond oil.

Another embodiment comprises cyclosporin A, DeSonic S-100®, and Almond oil.

Another embodiment comprises cyclosporin A, DeSonic S-114®, and Almond oil.

Another embodiment comprises cyclosporin A, DeSonic S-405®, and Almond oil.

Another embodiment comprises cyclosporin A, DeSonic S-45®, and Almond oil.

Another embodiment comprises cyclosporin A, DeSonic TDA-9®, and Almond oil.

Another embodiment comprises cyclosporin A, DeThox Acid O-14®, and Almond oil.

Another embodiment comprises cyclosporin A, DeThox Acid O-16®, and Almond oil.

Another embodiment comprises cyclosporin A, DeThox Acid O-9®, and Almond oil.

Another embodiment comprises cyclosporin A, DeThox Acid TO-14®, and Almond oil.

Another embodiment comprises cyclosporin A, DeThox Acid TO-16.5®, and Almond oil.

Another embodiment comprises cyclosporin A, DeThox Acid TO-8.5®, and Almond oil.

Another embodiment comprises cyclosporin A, Dur-Em 114®, and Almond oil.

Another embodiment comprises cyclosporin A, Dur-Em 117®, and Almond oil.

Another embodiment comprises cyclosporin A, Dur-Em 204®, and Almond oil.

Another embodiment comprises cyclosporin A, Dur-Em 207®, and Almond oil.

Another embodiment comprises cyclosporin A, Dur-Em 207E®, and Almond oil.

Another embodiment comprises cyclosporin A, Emerest 2704®, and Almond oil.

Another embodiment comprises cyclosporin A, Emerest 2712®, and Almond oil.

Another embodiment comprises cyclosporin A, Emerest 2715®, and Almond oil.

Another embodiment comprises cyclosporin A, Ethal EH-2®, and Almond oil.

Another embodiment comprises cyclosporin A, Ethal EH-5®, and Almond oil.

Another embodiment comprises cyclosporin A, Ethal OA-23®, and Almond oil.

Another embodiment comprises cyclosporin A, Ethosperse CA-20®, and Almond oil.

Another embodiment comprises cyclosporin A, Ethosperse LA-12®, and Almond oil.

Another embodiment comprises cyclosporin A, Ethosperse LA-23®, and Almond oil.

Another embodiment comprises cyclosporin A, Ethosperse LA-4®, and Almond oil.

Another embodiment comprises cyclosporin A, Ethox 3095®, and Almond oil.

Another embodiment comprises cyclosporin A, Ethox CO-16®, and Almond oil.

Another embodiment comprises cyclosporin A, Ethox CO-200®, and Almond oil.

Another embodiment comprises cyclosporin A, Ethox CO-25®, and Almond oil.

Another embodiment comprises cyclosporin A, Ethox CO-30®, and Almond oil.

Another embodiment comprises cyclosporin A, Ethox CO-36®, and Almond oil.

Another embodiment comprises cyclosporin A, Ethox CO-40®, and Almond oil.

Another embodiment comprises cyclosporin A, Ethox CO-5®, and Almond oil.

Another embodiment comprises cyclosporin A, Ethox DO-14®, and Almond oil.

Another embodiment comprises cyclosporin A, Ethox DO-9®, and Almond oil.

Another embodiment comprises cyclosporin A, Ethox MA-8®, and Almond oil.

Another embodiment comprises cyclosporin A, Ethox MI-14®, and Almond oil.

Another embodiment comprises cyclosporin A, Ethox ML-14®, and Almond oil.

Another embodiment comprises cyclosporin A, Ethox ML-5®, and Almond oil.

Another embodiment comprises cyclosporin A, Ethox MO-14®, and Almond oil.

Another embodiment comprises cyclosporin A, Ethox MO-9®, and Almond oil.

Another embodiment comprises cyclosporin A, Ethox MS-14®, and Almond oil.

Another embodiment comprises cyclosporin A, Ethox MS-23®, and Almond oil.

Another embodiment comprises cyclosporin A, Ethox MS-40®, and Almond oil.

Another embodiment comprises cyclosporin A, Ethox MS-8®, and Almond oil.

Another embodiment comprises cyclosporin A, Ethox TO-16®, and Almond oil.

Another embodiment comprises cyclosporin A, Ethox TO-8®, and Almond oil.

Another embodiment comprises cyclosporin A, Ethylan SN-12®, and Almond oil.

Another embodiment comprises cyclosporin A, Ethylan SN-70®, and Almond oil.

Another embodiment comprises cyclosporin A, Ethylan SN-90®, and Almond oil.

Another embodiment comprises cyclosporin A, Flexricin 13®, and Almond oil.

Another embodiment comprises cyclosporin A, Flexricin 15®, and Almond oil.

Another embodiment comprises cyclosporin A, Genapol 1261®, and Almond oil.

Another embodiment comprises cyclosporin A, Generol 122 E25®, and Almond oil.

Another embodiment comprises cyclosporin A, Glicepol 160®, and Almond oil.

Another embodiment comprises cyclosporin A, Glicepol 560®, and Almond oil.

Another embodiment comprises cyclosporin A, Glicepol GMS 20®, and Almond oil.

Another embodiment comprises cyclosporin A, Hetoxide BN-13®, and Almond oil.

Another embodiment comprises cyclosporin A, Hetoxide DNP-4®, and Almond oil.

Another embodiment comprises cyclosporin A, Hetoxide DNP-9.6®, and Almond oil.

Another embodiment comprises cyclosporin A, Hetoxide G-26®, and Almond oil.

Another embodiment comprises cyclosporin A, Hetoxide G-7®, and Almond oil.

Another embodiment comprises cyclosporin A, Hetoxide GT-20®, and Almond oil.

Another embodiment comprises cyclosporin A, Hetoxide HC-40®, and Almond oil.

Another embodiment comprises cyclosporin A, Hetoxol C-24®, and Almond oil.

Another embodiment comprises cyclosporin A, Hetoxol CA-10®, and Almond oil.

Another embodiment comprises cyclosporin A, Hetoxol CA-2®, and Almond oil.

Another embodiment comprises cyclosporin A, Hetoxol CA-20®, and Almond oil.

Another embodiment comprises cyclosporin A, Hetoxol CAWS®, and Almond oil.

Another embodiment comprises cyclosporin A, Hetoxol CD-3®, and Almond oil.

Another embodiment comprises cyclosporin A, Hetoxol CD-4®, and Almond oil.

Another embodiment comprises cyclosporin A, Hetoxol CS-15®, and Almond oil.

Another embodiment comprises cyclosporin A, Hetoxol CS-20®, and Almond oil.

Another embodiment comprises cyclosporin A, Hetoxol CS-20D®, and Almond oil.

Another embodiment comprises cyclosporin A, Hetoxol CS-25®, and Almond oil.

Another embodiment comprises cyclosporin A, Hetoxol CS-50®, and Almond oil.

Another embodiment comprises cyclosporin A, Hetoxol CS-9®, and Almond oil.

Another embodiment comprises cyclosporin A, Hetoxol CSA-15®, and Almond oil.

Another embodiment comprises cyclosporin A, Hetoxol D®, and Almond oil.

Another embodiment comprises cyclosporin A, Hetoxol G®, and Almond oil.

Another embodiment comprises cyclosporin A, Hetoxol J®, and Almond oil.

Another embodiment comprises cyclosporin A, Hetoxol L®, and Almond oil.

Another embodiment comprises cyclosporin A, Hetoxol L-23®, and Almond oil.

Another embodiment comprises cyclosporin A, Hetoxol L-4®, and Almond oil.

Another embodiment comprises cyclosporin A, Hetoxol L-9®, and Almond oil.

Another embodiment comprises cyclosporin A, Hetoxol LS-9®, and Almond oil.

Another embodiment comprises cyclosporin A, Hetoxol M-3®, and Almond oil.

Another embodiment comprises cyclosporin A, Hetoxol OA-10 Special®, and Almond oil.

Another embodiment comprises cyclosporin A, Hetoxol OA-20 Special®, and Almond oil.

Another embodiment comprises cyclosporin A, Hetoxol OA-3 Special®, and Almond oil.

Another embodiment comprises cyclosporin A, Hetoxol OA-5 Special®, and Almond oil.

Another embodiment comprises cyclosporin A, Hetoxol OL-10®, and Almond oil.

Another embodiment comprises cyclosporin A, Hetoxol OL-2®, and Almond oil.

Another embodiment comprises cyclosporin A, Hetoxol OL-23®, and Almond oil.

Another embodiment comprises cyclosporin A, Hetoxol OL-4®, and Almond oil.

Another embodiment comprises cyclosporin A, Hetoxol OL-40®, and Almond oil.

Another embodiment comprises cyclosporin A, Hetoxol OL-5®, and Almond oil.

Another embodiment comprises cyclosporin A, Hetoxol PLA®, and Almond oil.

Another embodiment comprises cyclosporin A, Hetoxol SP-15®, and Almond oil.

Another embodiment comprises cyclosporin A, Hetoxol STA-10®, and Almond oil.

Another embodiment comprises cyclosporin A, Hetoxol STA-2®, and Almond oil.

Another embodiment comprises cyclosporin A, Hetoxol STA-30®, and Almond oil.

Another embodiment comprises cyclosporin A, Hetoxol TD-12®, and Almond oil.

Another embodiment comprises cyclosporin A, Hetoxol TD-18®, and Almond oil.

Another embodiment comprises cyclosporin A, Hetoxol TD-3®, and Almond oil.

Another embodiment comprises cyclosporin A, Hetoxol TD-6®, and Almond oil.

Another embodiment comprises cyclosporin A, Hetoxol TD-9®, and Almond oil.

Another embodiment comprises cyclosporin A, Hetoxol TDEP-15®, and Almond oil.

Another embodiment comprises cyclosporin A, Hetoxol TDEP-63®, and Almond oil.

Another embodiment comprises cyclosporin A, Iconol 24-12®, and Almond oil.

Another embodiment comprises cyclosporin A, Iconol 24-23®, and Almond oil.

Another embodiment comprises cyclosporin A, Iconol 24-4®, and Almond oil.

Another embodiment comprises cyclosporin A, Iconol DA-4®, and Almond oil.

Another embodiment comprises cyclosporin A, Iconol DA-6®, and Almond oil.

Another embodiment comprises cyclosporin A, Iconol TDA-10®, and Almond oil.

Another embodiment comprises cyclosporin A, Iconol TDA-3®, and Almond oil.

Another embodiment comprises cyclosporin A, Iconol TDA-6®, and Almond oil.

Another embodiment comprises cyclosporin A, Iconol TDA-8-90%®, and Almond oil.

Another embodiment comprises cyclosporin A, Iconol TDA-9®, and Almond oil.

Another embodiment comprises cyclosporin A, Igepal CA-210®, and Almond oil.

Another embodiment comprises cyclosporin A, Igepal CA-407®, and Almond oil.

Another embodiment comprises cyclosporin A, Igepal CA-420®, and Almond oil.

Another embodiment comprises cyclosporin A, Igepal CA-520®, and Almond oil.

Another embodiment comprises cyclosporin A, Igepal CA-620®, and Almond oil.

Another embodiment comprises cyclosporin A, Igepal CA-630®, and Almond oil.

Another embodiment comprises cyclosporin A, Igepal CA-720®, and Almond oil.

Another embodiment comprises cyclosporin A, Igepal CA-877®, and Almond oil.

Another embodiment comprises cyclosporin A, Igepal CA-887®, and Almond oil.

Another embodiment comprises cyclosporin A, Igepal CA-897®, and Almond oil.

Another embodiment comprises cyclosporin A, Igepal CO-210®, and Almond oil.

Another embodiment comprises cyclosporin A, Igepal CO-430®, and Almond oil.

Another embodiment comprises cyclosporin A, Igepal CO-520®, and Almond oil.

Another embodiment comprises cyclosporin A, Igepal CO-530®, and Almond oil.

Another embodiment comprises cyclosporin A, Igepal CO-610®, and Almond oil.

Another embodiment comprises cyclosporin A, Igepal CO-630®, and Almond oil.

Another embodiment comprises cyclosporin A, Igepal CO-630 Special®, and Almond oil.

Another embodiment comprises cyclosporin A, Igepal CO-660®, and Almond oil.

Another embodiment comprises cyclosporin A, Igepal CO-710®, and Almond oil.

Another embodiment comprises cyclosporin A, Igepal CO-720®, and Almond oil.

Another embodiment comprises cyclosporin A, Igepal CO-730®, and Almond oil.

Another embodiment comprises cyclosporin A, Igepal CO-738®, and Almond oil.

Another embodiment comprises cyclosporin A, Igepal CO-850®, and Almond oil.

Another embodiment comprises cyclosporin A, Igepal CO-880®, and Almond oil.

Another embodiment comprises cyclosporin A, Igepal CO-887®, and Almond oil.

Another embodiment comprises cyclosporin A, Igepal CO-890®, and Almond oil.

Another embodiment comprises cyclosporin A, Igepal CO-897®, and Almond oil.

Another embodiment comprises cyclosporin A, Igepal CO-970®, and Almond oil.

Another embodiment comprises cyclosporin A, Igepal CO-977®, and Almond oil.

Another embodiment comprises cyclosporin A, Igepal CO-987®, and Almond oil.

Another embodiment comprises cyclosporin A, Igepal CO-990 FLK®, and Almond oil.

Another embodiment comprises cyclosporin A, Igepal CO-997®, and Almond oil.

Another embodiment comprises cyclosporin A, Igepal DM-430®, and Almond oil.

Another embodiment comprises cyclosporin A, Igepal DM-530®, and Almond oil.

Another embodiment comprises cyclosporin A, Igepal DM-710®, and Almond oil.

Another embodiment comprises cyclosporin A, Igepal DM-730®, and Almond oil.

Another embodiment comprises cyclosporin A, Igepal DM-880®, and Almond oil.

Another embodiment comprises cyclosporin A, Igepal DM-970 FLK®, and Almond oil.

Another embodiment comprises cyclosporin A, Igepal OD-410®, and Almond oil.

Another embodiment comprises cyclosporin A, Igepal RC-520®, and Almond oil.

Another embodiment comprises cyclosporin A, Igepal RC-620®, and Almond oil.

Another embodiment comprises cyclosporin A, Igepal RC-630®, and Almond oil.

Another embodiment comprises cyclosporin A, Lexemul CS-20®, and Almond oil.

Another embodiment comprises cyclosporin A, Lipo GMS 450®, and Almond oil.

Another embodiment comprises cyclosporin A, Lipo Polyglycol 1000®, and Almond oil.

Another embodiment comprises cyclosporin A, Lipo Polyglycol 200®, and Almond oil.

Another embodiment comprises cyclosporin A, Lipo Polyglycol 300®, and Almond oil.

Another embodiment comprises cyclosporin A, Lipo Polyglycol 3350®, and Almond oil.

Another embodiment comprises cyclosporin A, Lipo Polyglycol 400®, and Almond oil.

Another embodiment comprises cyclosporin A, Lipo Polyglycol 600®, and Almond oil.

Another embodiment comprises cyclosporin A, Lipocol C-10®, and Almond oil.

Another embodiment comprises cyclosporin A, Lipocol C-2®, and Almond oil.

Another embodiment comprises cyclosporin A, Lipocol C-20®, and Almond oil.

Another embodiment comprises cyclosporin A, Lipocol L-12®, and Almond oil.

Another embodiment comprises cyclosporin A, Lipocol L-23®, and Almond oil.

Another embodiment comprises cyclosporin A, Lipocol L-4®, and Almond oil.

Another embodiment comprises cyclosporin A, Lipocol O-10®, and Almond oil.

Another embodiment comprises cyclosporin A, Lipocol O-2®, and Almond oil.

Another embodiment comprises cyclosporin A, Lipocol O-20®, and Almond oil.

Another embodiment comprises cyclosporin A, Lipocol O-5®, and Almond oil.

Another embodiment comprises cyclosporin A, Lipocol S-10®, and Almond oil.

Another embodiment comprises cyclosporin A, Lipocol S-2®, and Almond oil.

Another embodiment comprises cyclosporin A, Lipocol S-20®, and Almond oil.

Another embodiment comprises cyclosporin A, Lipocol SC-15®, and Almond oil.

Another embodiment comprises cyclosporin A, Lipocol SC-20®, and Almond oil.

Another embodiment comprises cyclosporin A, Lipocol SC-4®, and Almond oil.

Another embodiment comprises cyclosporin A, Lipocol TD-12®, and Almond oil.

Another embodiment comprises cyclosporin A, Lumulse 40-O K®, and Almond oil.

Another embodiment comprises cyclosporin A, Lumulse 42-O K®, and Almond oil.

Another embodiment comprises cyclosporin A, Lumulse GMT-40®, and Almond oil.

Another embodiment comprises cyclosporin A, Lumulse PEG 1450®, and Almond oil.

Another embodiment comprises cyclosporin A, Lumulse PEG 1450 NF®, and Almond oil.

Another embodiment comprises cyclosporin A, Lumulse PEG 8000®, and Almond oil.

Another embodiment comprises cyclosporin A, Lumulse PGO®, and Almond oil.

Another embodiment comprises cyclosporin A, Lumulse POE (2) Oleyl Amine®, and Almond oil.

Another embodiment comprises cyclosporin A, Magrabar PGO-104®, and Almond oil.

Another embodiment comprises cyclosporin A, Mapeg 200 ML®, and Almond oil.

Another embodiment comprises cyclosporin A, Mapeg 400 MO®, and Almond oil.

Another embodiment comprises cyclosporin A, Mapeg 400 MOT®, and Almond oil.

Another embodiment comprises cyclosporin A, Mapeg 400DO®, and Almond oil.

Another embodiment comprises cyclosporin A, Mapeg 400DOT®, and Almond oil.

Another embodiment comprises cyclosporin A, Mapeg 600DOT®, and Almond oil.

Another embodiment comprises cyclosporin A, Marlox B 24/80®, and Almond oil.

Another embodiment comprises cyclosporin A, Marlox FK 86®, and Almond oil.

Another embodiment comprises cyclosporin A, Marlox MO 154®, and Almond oil.

Another embodiment comprises cyclosporin A, Mazox CDA CG®, and Almond oil.

Another embodiment comprises cyclosporin A, Monoglycerides Glyceryl Monestearate®, and Almond oil.

Another embodiment comprises cyclosporin A, Myrj 45®, and Almond oil.

Another embodiment comprises cyclosporin A, Myrj 52®, and Almond oil.

Another embodiment comprises cyclosporin A, Myrj 52S®, and Almond oil.

Another embodiment comprises cyclosporin A, Myrj 53®, and Almond oil.

Another embodiment comprises cyclosporin A, Myrj 59®, and Almond oil.

Another embodiment comprises cyclosporin A, Norfox NP-12®, and Almond oil.

Another embodiment comprises cyclosporin A, Norfox NP-4®, and Almond oil.

Another embodiment comprises cyclosporin A, Norfox NP-6®, and Almond oil.

Another embodiment comprises cyclosporin A, Norfox NP-887®, and Almond oil.

Another embodiment comprises cyclosporin A, Norfox NP-9®, and Almond oil.

Another embodiment comprises cyclosporin A, Norfox NP-977®, and Almond oil.

Another embodiment comprises cyclosporin A, Norfox OP-100®, and Almond oil.

Another embodiment comprises cyclosporin A, Norfox OP-102®, and Almond oil.

Another embodiment comprises cyclosporin A, Norfox OP-114®, and Almond oil.

Another embodiment comprises cyclosporin A, Norfox OP-45®, and Almond oil.

Another embodiment comprises cyclosporin A, Pegosperse 100-L®, and Almond oil.

Another embodiment comprises cyclosporin A, Pegosperse 100-O®, and Almond oil.

Another embodiment comprises cyclosporin A, Pegosperse 100-S®, and Almond oil.

Another embodiment comprises cyclosporin A, Pegosperse 1500-MS®, and Almond oil.

Another embodiment comprises cyclosporin A, Pegosperse 1750-MS®, and Almond oil.

Another embodiment comprises cyclosporin A, Pegosperse 200 DL®, and Almond oil.

Another embodiment comprises cyclosporin A, Pegosperse 200-ML®, and Almond oil.

Another embodiment comprises cyclosporin A, Pegosperse 400-DL®, and Almond oil.

Another embodiment comprises cyclosporin A, Pegosperse 400-DO®, and Almond oil.

Another embodiment comprises cyclosporin A, Pegosperse 400-DS®, and Almond oil.

Another embodiment comprises cyclosporin A, Pegosperse 400-ML®, and Almond oil.

Another embodiment comprises cyclosporin A, Pegosperse 400-MO®, and Almond oil.

Another embodiment comprises cyclosporin A, Pegosperse 400-MOT®, and Almond oil.

Another embodiment comprises cyclosporin A, Pegosperse 400-MS®, and Almond oil.

Another embodiment comprises cyclosporin A, Pegosperse 50-DS®, and Almond oil.

Another embodiment comprises cyclosporin A, Pegosperse 50-MS®, and Almond oil.

Another embodiment comprises cyclosporin A, Pegosperse 600-ML®, and Almond oil.

Another embodiment comprises cyclosporin A, Pegosperse 600-MS®, and Almond oil.

Another embodiment comprises cyclosporin A, Plurafac A-38®, and Almond oil.

Another embodiment comprises cyclosporin A, Plurafac B-25-5®, and Almond oil.

Another embodiment comprises cyclosporin A, Plurafac B-26®, and Almond oil.

Another embodiment comprises cyclosporin A, Plurafac D-25®, and Almond oil.

Another embodiment comprises cyclosporin A, Plurafac LF 131®, and Almond oil.

Another embodiment comprises cyclosporin A, Plurafac LF 4030®, and Almond oil.

Another embodiment comprises cyclosporin A, Plurafac LF 7000®, and Almond oil.

Another embodiment comprises cyclosporin A, Plurafac RA-20®, and Almond oil.

Another embodiment comprises cyclosporin A, Plurafac RA-30®, and Almond oil.

Another embodiment comprises cyclosporin A, Plurafac RA-40®, and Almond oil.

Another embodiment comprises cyclosporin A, Pluronic 10R5®, and Almond oil.

Another embodiment comprises cyclosporin A, Pluronic 17R2®, and Almond oil.

Another embodiment comprises cyclosporin A, Pluronic 17R4®, and Almond oil.

Another embodiment comprises cyclosporin A, Pluronic 25R2®, and Almond oil.

Another embodiment comprises cyclosporin A, Pluronic 25R4®, and Almond oil.

Another embodiment comprises cyclosporin A, Pluronic 31R1®, and Almond oil.

Another embodiment comprises cyclosporin A, Pluronic F108®, and Almond oil.

Another embodiment comprises cyclosporin A, Pluronic F127®, and Almond oil.

Another embodiment comprises cyclosporin A, Pluronic F38®, and Almond oil.

Another embodiment comprises cyclosporin A, Pluronic F68®, and Almond oil.

Another embodiment comprises cyclosporin A, Pluronic F68LF®, and Almond oil.

Another embodiment comprises cyclosporin A, Pluronic F77®, and Almond oil.

Another embodiment comprises cyclosporin A, Pluronic F87®, and Almond oil.

Another embodiment comprises cyclosporin A, Pluronic F88®, and Almond oil.

Another embodiment comprises cyclosporin A, Pluronic F98®, and Almond oil.

Another embodiment comprises cyclosporin A, Pluronic L10®, and Almond oil.

Another embodiment comprises cyclosporin A, Pluronic L101®, and Almond oil.

Another embodiment comprises cyclosporin A, Pluronic L121®, and Almond oil.

Another embodiment comprises cyclosporin A, Pluronic L31®, and Almond oil.

Another embodiment comprises cyclosporin A, Pluronic L35®, and Almond oil.

Another embodiment comprises cyclosporin A, Pluronic L43®, and Almond oil.

Another embodiment comprises cyclosporin A, Pluronic L44®, and Almond oil.

Another embodiment comprises cyclosporin A, Pluronic L61®, and Almond oil.

Another embodiment comprises cyclosporin A, Pluronic L62®, and Almond oil.

Another embodiment comprises cyclosporin A, Pluronic L62D®, and Almond oil.

Another embodiment comprises cyclosporin A, Pluronic L62LF®, and Almond oil.

Another embodiment comprises cyclosporin A, Pluronic L64®, and Almond oil.

Another embodiment comprises cyclosporin A, Pluronic L81®, and Almond oil.

Another embodiment comprises cyclosporin A, Pluronic L92®, and Almond oil.

Another embodiment comprises cyclosporin A, Pluronic P103®, and Almond oil.

Another embodiment comprises cyclosporin A, Pluronic P104®, and Almond oil.

Another embodiment comprises cyclosporin A, Pluronic P105®, and Almond oil.

Another embodiment comprises cyclosporin A, Pluronic P123®, and Almond oil.

Another embodiment comprises cyclosporin A, Pluronic P65®, and Almond oil.

Another embodiment comprises cyclosporin A, Pluronic P84®, and Almond oil.

Another embodiment comprises cyclosporin A, Pluronic P85®, and Almond oil.

Another embodiment comprises cyclosporin A, Polyaldo 10-2-P®, and Almond oil.

Another embodiment comprises cyclosporin A, Polyaldo DGDO®, and Almond oil.

Another embodiment comprises cyclosporin A, Polyaldo HGDS®, and Almond oil.

Another embodiment comprises cyclosporin A, Polyaldo TGMS®, and Almond oil.

Another embodiment comprises cyclosporin A, Protegin V®, and Almond oil.

Another embodiment comprises cyclosporin A, Protegin W®, and Almond oil.

Another embodiment comprises cyclosporin A, Protegin WX®, and Almond oil.

Another embodiment comprises cyclosporin A, Protegin XV®, and Almond oil.

Another embodiment comprises cyclosporin A, Rhodasurf BC-420®, and Almond oil.

Another embodiment comprises cyclosporin A, Rhodasurf BC-610®, and Almond oil.

Another embodiment comprises cyclosporin A, Rhodasurf BC-630®, and Almond oil.

Another embodiment comprises cyclosporin A, Rhodasurf BC-720®, and Almond oil.

Another embodiment comprises cyclosporin A, Rhodasurf BC-840®, and Almond oil.

Another embodiment comprises cyclosporin A, Rhodasurf L 1®, and Almond oil.

Another embodiment comprises cyclosporin A, Rhodasurf L 12®, and Almond oil.

Another embodiment comprises cyclosporin A, Rhodasurf L 2®, and Almond oil.

Another embodiment comprises cyclosporin A, Rhodasurf L 20®, and Almond oil.

Another embodiment comprises cyclosporin A, Rhodasurf L 3®, and Almond oil.

Another embodiment comprises cyclosporin A, Rhodasurf L 30®, and Almond oil.

Another embodiment comprises cyclosporin A, Rhodasurf L 4®, and Almond oil.

Another embodiment comprises cyclosporin A, Rhodasurf L 50®, and Almond oil.

Another embodiment comprises cyclosporin A, Rhodasurf L 7/90®, and Almond oil.

Another embodiment comprises cyclosporin A, Rhodasurf LA-3®, and Almond oil.

Another embodiment comprises cyclosporin A, Rhodasurf LA-7®, and Almond oil.

Another embodiment comprises cyclosporin A, Rhodasurf LAN-23®, and Almond oil.

Another embodiment comprises cyclosporin A, Rhodasurf ON-870®, and Almond oil.

Another embodiment comprises cyclosporin A, Rhodasurf ON-877®, and Almond oil.

Another embodiment comprises cyclosporin A, Rhodasurf TDA-8/5®, and Almond oil.

Another embodiment comprises cyclosporin A, Ritapro 100®, and Almond oil.

Another embodiment comprises cyclosporin A, Ritapro 165®, and Almond oil.

Another embodiment comprises cyclosporin A, Ritapro 200®, and Almond oil.

Another embodiment comprises cyclosporin A, Ritapro 300®, and Almond oil.

Another embodiment comprises cyclosporin A, Ritoleth 10®, and Almond oil.

Another embodiment comprises cyclosporin A, Ritoleth 2®, and Almond oil.

Another embodiment comprises cyclosporin A, Ritoleth 20®, and Almond oil.

Another embodiment comprises cyclosporin A, Ritoleth 5®, and Almond oil.

Another embodiment comprises cyclosporin A, Santone 10-10-0®, and Almond oil.

Another embodiment comprises cyclosporin A, Santone 3-1-S XTR®, and Almond oil.

Another embodiment comprises cyclosporin A, Santone 3-1-SH®, and Almond oil.

Another embodiment comprises cyclosporin A, Santone 8-1-O®, and Almond oil.

Another embodiment comprises cyclosporin A, Solulan 16®, and Almond oil.

Another embodiment comprises cyclosporin A, Solulan 75®, and Almond oil.

Another embodiment comprises cyclosporin A, Solulan C-24®, and Almond oil.

Another embodiment comprises cyclosporin A, Solulan L-575®, and Almond oil.

Another embodiment comprises cyclosporin A, Standamox CAW®, and Almond oil.

Another embodiment comprises cyclosporin A, Surfine AZI-A®, and Almond oil.

Another embodiment comprises cyclosporin A, Surfonic CO-15®, and Almond oil.

Another embodiment comprises cyclosporin A, Surfonic CO-25®, and Almond oil.

Another embodiment comprises cyclosporin A, Surfonic CO-30®, and Almond oil.

Another embodiment comprises cyclosporin A, Surfonic CO-36®, and Almond oil.

Another embodiment comprises cyclosporin A, Surfonic CO-42®, and Almond oil.

Another embodiment comprises cyclosporin A, Surfonic E400-MO®, and Almond oil.

Another embodiment comprises cyclosporin A, Surfonic L12-3®, and Almond oil.

Another embodiment comprises cyclosporin A, Surfonic L12-6®, and Almond oil.

Another embodiment comprises cyclosporin A, Surfonic L12-8®, and Almond oil.

Another embodiment comprises cyclosporin A, Surfonic L24-12®, and Almond oil.

Another embodiment comprises cyclosporin A, Surfonic L24-2®, and Almond oil.

Another embodiment comprises cyclosporin A, Surfonic L24-22®, and Almond oil.

Another embodiment comprises cyclosporin A, Surfonic L24-3®, and Almond oil.

Another embodiment comprises cyclosporin A, Surfonic L24-4®, and Almond oil.

Another embodiment comprises cyclosporin A, Surfonic L24-5®, and Almond oil.

Another embodiment comprises cyclosporin A, Surfonic L24-7®, and Almond oil.

Another embodiment comprises cyclosporin A, Surfonic L24-7.1®, and Almond oil.

Another embodiment comprises cyclosporin A, Surfonic L24-9®, and Almond oil.

Another embodiment comprises cyclosporin A, Surfonic LF-17®, and Almond oil.

Another embodiment comprises cyclosporin A, Surfonic LF-18®, and Almond oil.

Another embodiment comprises cyclosporin A, Surfonic LF-37®, and Almond oil.

Another embodiment comprises cyclosporin A, Surfonic LF-41®, and Almond oil.

Another embodiment comprises cyclosporin A, Surfonic LF-42®, and Almond oil.

Another embodiment comprises cyclosporin A, Surfonic LF-47®, and Almond oil.

Another embodiment comprises cyclosporin A, Surfonic LF-68®, and Almond oil.

Another embodiment comprises cyclosporin A, Surfonic N-10®, and Almond oil.

Another embodiment comprises cyclosporin A, Surfonic N-100®, and Almond oil.

Another embodiment comprises cyclosporin A, Surfonic N-102®, and Almond oil.

Another embodiment comprises cyclosporin A, Surfonic N-120®, and Almond oil.

Another embodiment comprises cyclosporin A, Surfonic N-150®, and Almond oil.

Another embodiment comprises cyclosporin A, Surfonic N-200®, and Almond oil.

Another embodiment comprises cyclosporin A, Surfonic N-300®, and Almond oil.

Another embodiment comprises cyclosporin A, Surfonic N-31.5®, and Almond oil.

Another embodiment comprises cyclosporin A, Surfonic N-40®, and Almond oil.

Another embodiment comprises cyclosporin A, Surfonic N-400®, and Almond oil.

Another embodiment comprises cyclosporin A, Surfonic N-60®, and Almond oil.

Another embodiment comprises cyclosporin A, Surfonic N-85®, and Almond oil.

Another embodiment comprises cyclosporin A, Surfonic N-95®, and Almond oil.

Another embodiment comprises cyclosporin A, Surfonic NB-18®, and Almond oil.

Another embodiment comprises cyclosporin A, Surfonic NB-23®, and Almond oil.

Another embodiment comprises cyclosporin A, Surfonic NB-25®, and Almond oil.

Another embodiment comprises cyclosporin A, Surfonic NB-307®, and Almond oil.

Another embodiment comprises cyclosporin A, Surfonic NB-31®, and Almond oil.

Another embodiment comprises cyclosporin A, Surfonic NB-41®, and Almond oil.

Another embodiment comprises cyclosporin A, Surfonic NB-43®, and Almond oil.

Another embodiment comprises cyclosporin A, Surfonic OP-100®, and Almond oil.

Another embodiment comprises cyclosporin A, Surfonic OP-120®, and Almond oil.

Another embodiment comprises cyclosporin A, Surfonic OP-15®, and Almond oil.

Another embodiment comprises cyclosporin A, Surfonic OP-35®, and Almond oil.

Another embodiment comprises cyclosporin A, Surfonic OP-70®, and Almond oil.

Another embodiment comprises cyclosporin A, Surfonic P1®, and Almond oil.

Another embodiment comprises cyclosporin A, Surfonic P3®, and Almond oil.

Another embodiment comprises cyclosporin A, Surfonic P5®, and Almond oil.

Another embodiment comprises cyclosporin A, Surfonic P6®, and Almond oil.

Another embodiment comprises cyclosporin A, Surfonic POA-17R2®, and Almond oil.

Another embodiment comprises cyclosporin A, Surtonic POA-25R2®, and Almond oil.

Another embodiment comprises cyclosporin A, Surfonic TDA-11®, and Almond oil.

Another embodiment comprises cyclosporin A, Surfonic TDA-3B®, and Almond oil.

Another embodiment comprises cyclosporin A, Surfonic TDA-6®, and Almond oil.

Another embodiment comprises cyclosporin A, Surfonic TDA-6/88®, and Almond oil.

Another embodiment comprises cyclosporin A, Surfonic TDA-8®, and Almond oil.

Another embodiment comprises cyclosporin A, Surfonic TDA-8/90®, and Almond oil.

Another embodiment comprises cyclosporin A, Surfonic TDA-9®, and Almond oil.

Another embodiment comprises cyclosporin A, Surfonic TX-CCN®, and Almond oil.

Another embodiment comprises cyclosporin A, Tagat CH 40®, and Almond oil.

Another embodiment comprises cyclosporin A, Tagat CH 60®, and Almond oil.

Another embodiment comprises cyclosporin A, Tagat L 2®, and Almond oil.

Another embodiment comprises cyclosporin A, Tagat S®, and Almond oil.

Another embodiment comprises cyclosporin A, Tagat S 2®, and Almond oil.

Another embodiment comprises cyclosporin A, Tally 100 Plus®, and Almond oil.

Another embodiment comprises cyclosporin A, T-Det A106®, and Almond oil.

Another embodiment comprises cyclosporin A, T-Det A109®, and Almond oil.

Another embodiment comprises cyclosporin A, T-Det A136®, and Almond oil.

Another embodiment comprises cyclosporin A, T-Det A139®, and Almond oil.

Another embodiment comprises cyclosporin A, T-Det A2412®, and Almond oil.

Another embodiment comprises cyclosporin A, T-Det A243®, and Almond oil.

Another embodiment comprises cyclosporin A, T-Det A247®, and Almond oil.

Another embodiment comprises cyclosporin A, T-Det A249®, and Almond oil.

Another embodiment comprises cyclosporin A, T-Det A467®, and Almond oil.

Another embodiment comprises cyclosporin A, T-Det COE®, and Almond oil.

Another embodiment comprises cyclosporin A, T-Det DD-10®, and Almond oil.

Another embodiment comprises cyclosporin A, T-Det DD-5®, and Almond oil.

Another embodiment comprises cyclosporin A, T-Det DD-7®, and Almond oil.

Another embodiment comprises cyclosporin A, T-Det EPO-104®, and Almond oil.

Another embodiment comprises cyclosporin A, T-Det EPO-61®, and Almond oil.

Another embodiment comprises cyclosporin A, T-Det EPO-62®, and Almond oil.

Another embodiment comprises cyclosporin A, T-Det EPO-64®, and Almond oil.

Another embodiment comprises cyclosporin A, T-Det N-1.5®, and Almond oil.

Another embodiment comprises cyclosporin A, T-Det N-10.5®, and Almond oil.

Another embodiment comprises cyclosporin A, T-Det N-100®, and Almond oil.

Another embodiment comprises cyclosporin A, T-Det N-1007®, and Almond oil.

Another embodiment comprises cyclosporin A, T-Det N-12®, and Almond oil.

Another embodiment comprises cyclosporin A, T-Det N-14®, and Almond oil.

Another embodiment comprises cyclosporin A, T-Det N-20®, and Almond oil.

Another embodiment comprises cyclosporin A, T-Det N-30®, and Almond oil.

Another embodiment comprises cyclosporin A, T-Det N-307®, and Almond oil.

Another embodiment comprises cyclosporin A, T-Det N-4®, and Almond oil.

Another embodiment comprises cyclosporin A, T-Det N-40®, and Almond oil.

Another embodiment comprises cyclosporin A, T-Det N-407®, and Almond oil.

Another embodiment comprises cyclosporin A, T-Det N-50®, and Almond oil.

Another embodiment comprises cyclosporin A, T-Det N-507®, and Almond oil.

Another embodiment comprises cyclosporin A, T-Det N-6®, and Almond oil.

Another embodiment comprises cyclosporin A, T-Det N-70®, and Almond oil.

Another embodiment comprises cyclosporin A, T-Det N-8®, and Almond oil.

Another embodiment comprises cyclosporin A, T-Det N-9.5®, and Almond oil.

Another embodiment comprises cyclosporin A, T-Det O-12®, and Almond oil.

Another embodiment comprises cyclosporin A, T-Det O-165®, and Almond oil.

Another embodiment comprises cyclosporin A, T-Det O-307®, and Almond oil.

Another embodiment comprises cyclosporin A, T-Det O-40®, and Almond oil.

Another embodiment comprises cyclosporin A, T-Det O-407®, and Almond oil.

Another embodiment comprises cyclosporin A, T-Det O-6®, and Almond oil.

Another embodiment comprises cyclosporin A, T-Det O-8®, and Almond oil.

Another embodiment comprises cyclosporin A, T-Det O-9®, and Almond oil.

Another embodiment comprises cyclosporin A, Tegin®, and Almond oil.

Another embodiment comprises cyclosporin A, Tergitol 15-S-12®, and Almond oil.

Another embodiment comprises cyclosporin A, Tergitol 15-S-15®, and Almond oil.

Another embodiment comprises cyclosporin A, Tergitol 15-S-20®, and Almond oil.

Another embodiment comprises cyclosporin A, Tergitol 15-S-20 (80AQ)®, and Almond oil.

Another embodiment comprises cyclosporin A, Tergitol 15-S-3®, and Almond oil.

Another embodiment comprises cyclosporin A, Tergitol 15-S-30®, and Almond oil.

Another embodiment comprises cyclosporin A, Tergitol 15-S-40®, and Almond oil.

Another embodiment comprises cyclosporin A, Tergitol 15-S-40 (70AQ)®, and Almond oil.

Another embodiment comprises cyclosporin A, Tergitol 15-S-5®, and Almond oil.

Another embodiment comprises cyclosporin A, Tergitol 15-S-7®, and Almond oil.

Another embodiment comprises cyclosporin A, Tergitol 15-S-9®, and Almond oil.

Another embodiment comprises cyclosporin A, Tergitol L-101®, and Almond oil.

Another embodiment comprises cyclosporin A, Tergitol L-61®, and Almond oil.

Another embodiment comprises cyclosporin A, Tergitol L-62®, and Almond oil.

Another embodiment comprises cyclosporin A, Tergitol L-64®, and Almond oil.

Another embodiment comprises cyclosporin A, Tergitol L-81®, and Almond oil.

Another embodiment comprises cyclosporin A, Tergitol NP-10®, and Almond oil.

Another embodiment comprises cyclosporin A, Tergitol NP-11®, and Almond oil.

Another embodiment comprises cyclosporin A, Tergitol NP-12®, and Almond oil.

Another embodiment comprises cyclosporin A, Tergitol NP-13®, and Almond oil.

Another embodiment comprises cyclosporin A, Tergitol NP-15®, and Almond oil.

Another embodiment comprises cyclosporin A, Tergitol NP-30®, and Almond oil.

Another embodiment comprises cyclosporin A, Tergitol NP-30 (70 AQ)®, and Almond oil.

Another embodiment comprises cyclosporin A, Tergitol NP-4®, and Almond oil.

Another embodiment comprises cyclosporin A, Tergitol NP-40®, and Almond oil.

Another embodiment comprises cyclosporin A, Tergitol NP-40 (70 AQ)®, and Almond oil.

Another embodiment comprises cyclosporin A, Tergitol NP-50 (70 AQ)®, and Almond oil.

Another embodiment comprises cyclosporin A, Tergitol NP-55 (70 AQ)®, and Almond oil.

Another embodiment comprises cyclosporin A, Tergitol NP-6®, and Almond oil.

Another embodiment comprises cyclosporin A, Tergitol NP-7®, and Almond oil.

Another embodiment comprises cyclosporin A, Tergitol NP-70 (70 AQ)®, and Almond oil.

Another embodiment comprises cyclosporin A, Tergitol NP-8®, and Almond oil.

Another embodiment comprises cyclosporin A, Tergitol NP-9®, and Almond oil.

Another embodiment comprises cyclosporin A, Tergitol NP-9.5®, and Almond oil.

Another embodiment comprises cyclosporin A, Tergitol TMN-10®, and Almond oil.

Another embodiment comprises cyclosporin A, Tergitol TMN-3®, and Almond oil.

Another embodiment comprises cyclosporin A, Tergitol TMN-6®, and Almond oil.

Another embodiment comprises cyclosporin A, Tergitol XD®, and Almond oil.

Another embodiment comprises cyclosporin A, Tergitol XH®, and Almond oil.

Another embodiment comprises cyclosporin A, Tergitol XJ®, and Almond oil.

Another embodiment comprises cyclosporin A, Tetronic 1107®, and Almond oil.

Another embodiment comprises cyclosporin A, Tetronic 1307®, and Almond oil.

Another embodiment comprises cyclosporin A, Tetronic 150R1®, and Almond oil.

Another embodiment comprises cyclosporin A, Tetronic 304®, and Almond oil.

Another embodiment comprises cyclosporin A, Tetronic 701®, and Almond oil.

Another embodiment comprises cyclosporin A, Tetronic 901®, and Almond oil.

Another embodiment comprises cyclosporin A, Tetronic 904®, and Almond oil.

Another embodiment comprises cyclosporin A, Tetronic 908®, and Almond oil.

Another embodiment comprises cyclosporin A, Tetronic 90R4®, and Almond oil.

Another embodiment comprises cyclosporin A, Tomadol 1-3®, and Almond oil.

Another embodiment comprises cyclosporin A, Tomadol 1-5®, and Almond oil.

Another embodiment comprises cyclosporin A, Tomadol 1-7®, and Almond oil.

Another embodiment comprises cyclosporin A, Tomadol 23-1®, and Almond oil.

Another embodiment comprises cyclosporin A, Tomadol 23-3®, and Almond oil.

Another embodiment comprises cyclosporin A, Tomadol 23-5®, and Almond oil.

Another embodiment comprises cyclosporin A, Tomadol 23-6.5®, and Almond oil.

Another embodiment comprises cyclosporin A, Tomadol 25-12®, and Almond oil.

Another embodiment comprises cyclosporin A, Tomadol 25-3®, and Almond oil.

Another embodiment comprises cyclosporin A, Tomadol 25-7®, and Almond oil.

Another embodiment comprises cyclosporin A, Tomadol 25-9®, and Almond oil.

Another embodiment comprises cyclosporin A, Tomadol 45-13®, and Almond oil.

Another embodiment comprises cyclosporin A, Tomadol 45-2.5®, and Almond oil.

Another embodiment comprises cyclosporin A, Tomadol 45-7®, and Almond oil.

Another embodiment comprises cyclosporin A, Tomadol 91-2.5®, and Almond oil.

Another embodiment comprises cyclosporin A, Tomadol 91-6®, and Almond oil.

Another embodiment comprises cyclosporin A, Tomadol 91-8®, and Almond oil.

Another embodiment comprises cyclosporin A, Triton X-100®, and Almond oil.

Another embodiment comprises cyclosporin A, Triton X-102®, and Almond oil.

Another embodiment comprises cyclosporin A, Triton X-114®, and Almond oil.

Another embodiment comprises cyclosporin A, Triton X-15®, and Almond oil.

Another embodiment comprises cyclosporin A, Triton X-165 (70%)®, and Almond oil.

Another embodiment comprises cyclosporin A, Triton X-207®, and Almond oil.

Another embodiment comprises cyclosporin A, Triton X-305 (70%)®, and Almond oil.

Another embodiment comprises cyclosporin A, Triton X-35®, and Almond oil.

Another embodiment comprises cyclosporin A, Triton X-405 (70%)®, and Almond oil.

Another embodiment comprises cyclosporin A, Triton X-45®, and Almond oil.

Another embodiment comprises cyclosporin A, Triton X-705 (70%)®, and Almond oil.

Another embodiment comprises cyclosporin A, Volpo S-10®, and Almond oil.

Another embodiment comprises cyclosporin A, Volpo S-2®, and Almond oil.

Another embodiment comprises cyclosporin A, Volpo S-20®, and Almond oil.

Another embodiment comprises cyclosporin A, Volpo-10®, and Almond oil.

Another embodiment comprises cyclosporin A, Volpo-20®, and Almond oil.

Another embodiment comprises cyclosporin A, Volpo-3®, and Almond oil.

Another embodiment comprises cyclosporin A, Volpo-5®, and Almond oil.

Another embodiment comprises cyclosporin A, Abex 2515®, and Corn oil.

Another embodiment comprises cyclosporin A, Abex 2525/40®, and Corn oil.

Another embodiment comprises cyclosporin A, Abex 2535®, and Corn oil.

Another embodiment comprises cyclosporin A, Abex 2545®, and Corn oil.

Another embodiment comprises cyclosporin A, Acconon E®, and Corn oil.

Another embodiment comprises cyclosporin A, Alfonic 1216CO-9®, and Corn oil.

Another embodiment comprises cyclosporin A, Alfonic 1412-3®, and Corn oil.

Another embodiment comprises cyclosporin A, Alfonic TDA-12®, and Corn oil.

Another embodiment comprises cyclosporin A, Alfonic TDA-3®, and Corn oil.

Another embodiment comprises cyclosporin A, Alfonic TDA-4®, and Corn oil.

Another embodiment comprises cyclosporin A, Alfonic TDA-6®, and Corn oil.

Another embodiment comprises cyclosporin A, Alfonic TDA-7®, and Corn oil.

Another embodiment comprises cyclosporin A, Alfonic TDA-8®, and Corn oil.

Another embodiment comprises cyclosporin A, Alfonic TDA-9®, and Corn oil.

Another embodiment comprises cyclosporin A, Alkamuls 400-DO®, and Corn oil.

Another embodiment comprises cyclosporin A, Alkamuls 400-MO/E®, and Corn oil.

Another embodiment comprises cyclosporin A, Ameroxol OE-10®, and Corn oil.

Another embodiment comprises cyclosporin A, Ameroxol OE-2®, and Corn oil.

Another embodiment comprises cyclosporin A, Ameroxol OE-20®, and Corn oil.

Another embodiment comprises cyclosporin A, Armix 176®, and Corn oil.

Another embodiment comprises cyclosporin A, Armix 180-C®, and Corn oil.

Another embodiment comprises cyclosporin A, Armix 183®, and Corn oil.

Another embodiment comprises cyclosporin A, Brij 35®, and Corn oil.

Another embodiment comprises cyclosporin A, Brij 35 Liquid®, and Corn oil.

Another embodiment comprises cyclosporin A, Brij 35 SP®, and Corn oil.

Another embodiment comprises cyclosporin A, Canasol MJ 2109®, and Corn oil.

Another embodiment comprises cyclosporin A, Canasol MJ 52®, and Corn oil.

Another embodiment comprises cyclosporin A, Canasol R 3600®, and Corn oil.

Another embodiment comprises cyclosporin A, Canasol R 3603®, and Corn oil.

Another embodiment comprises cyclosporin A, Canasol R 4000 H®, and Corn oil.

Another embodiment comprises cyclosporin A, Cedepal CA-210®, and Corn oil.

Another embodiment comprises cyclosporin A, Cedepal CA-520®, and Corn oil.

Another embodiment comprises cyclosporin A, Cedepal CA-630®, and Corn oil.

Another embodiment comprises cyclosporin A, Cedepal CA-720®, and Corn oil.

Another embodiment comprises cyclosporin A, Cedepal CA-890®, and Corn oil.

Another embodiment comprises cyclosporin A, Cedepal CA-897®, and Corn oil.

Another embodiment comprises cyclosporin A, Cedepal CO-210®, and Corn oil.

Another embodiment comprises cyclosporin A, Cedepal CO-430®, and Corn oil.

Another embodiment comprises cyclosporin A, Cedepal CO-530®, and Corn oil.

Another embodiment comprises cyclosporin A, Cedepal CO-630®, and Corn oil.

Another embodiment comprises cyclosporin A, Cedepal CO-710®, and Corn oil.

Another embodiment comprises cyclosporin A, Cedepal CO-730®, and Corn oil.

Another embodiment comprises cyclosporin A, Cedepal CO-880®, and Corn oil.

Another embodiment comprises cyclosporin A, Cedepal CO-887®, and Corn oil.

Another embodiment comprises cyclosporin A, Cedepal CO-890®, and Corn oil.

Another embodiment comprises cyclosporin A, Cedepal CO-897®, and Corn oil.

Another embodiment comprises cyclosporin A, Cedepal CO-970®, and Corn oil.

Another embodiment comprises cyclosporin A, Cedepal CO-977®, and Corn oil.

Another embodiment comprises cyclosporin A, Cedepal CO-990®, and Corn oil.

Another embodiment comprises cyclosporin A, Cedepal CO-997®, and Corn oil.

Another embodiment comprises cyclosporin A, Chemal BP 261®, and Corn oil.

Another embodiment comprises cyclosporin A, Chemal BP 262®, and Corn oil.

Another embodiment comprises cyclosporin A, Chemal BP 3172®, and Corn oil.

Another embodiment comprises cyclosporin A, Chemal BP 3174®, and Corn oil.

Another embodiment comprises cyclosporin A, Chemal DA-4®, and Corn oil.

Another embodiment comprises cyclosporin A, Chemal DA-6®, and Corn oil.

Another embodiment comprises cyclosporin A, Chemal DA-9®, and Corn oil.

Another embodiment comprises cyclosporin A, Chemal LA-23®, and Corn oil.

Another embodiment comprises cyclosporin A, Chemal LA-4®, and Corn oil.

Another embodiment comprises cyclosporin A, Chemal LA-9®, and Corn oil.

Another embodiment comprises cyclosporin A, Chemal OA-20G®, and Corn oil.

Another embodiment comprises cyclosporin A, Chemal OA-5®, and Corn oil.

Another embodiment comprises cyclosporin A, Chemal OA-9®, and Corn oil.

Another embodiment comprises cyclosporin A, Chemal TDA-12®, and Corn oil.

Another embodiment comprises cyclosporin A, Chemal TDA-15®, and Corn oil.

Another embodiment comprises cyclosporin A, Chemal TDA-3®, and Corn oil.

Another embodiment comprises cyclosporin A, Chemal TDA-6®, and Corn oil.

Another embodiment comprises cyclosporin A, Chemal TDA-9®, and Corn oil.

Another embodiment comprises cyclosporin A, Chemax CO-16®, and Corn oil.

Another embodiment comprises cyclosporin A, Chemax CO-200/50®, and Corn oil.

Another embodiment comprises cyclosporin A, Chemax CO-25®, and Corn oil.

Another embodiment comprises cyclosporin A, Chemax CO-30®, and Corn oil.

Another embodiment comprises cyclosporin A, Chemax CO-36®, and Corn oil.

Another embodiment comprises cyclosporin A, Chemax CO-40®, and Corn oil.

Another embodiment comprises cyclosporin A, Chemax CO-5®, and Corn oil.

Another embodiment comprises cyclosporin A, Chemax CO-80®, and Corn oil.

Another embodiment comprises cyclosporin A, Chemax DNP-150/50®, and Corn oil.

Another embodiment comprises cyclosporin A, Chemax DNP-18®, and Corn oil.

Another embodiment comprises cyclosporin A, Chemax DNP-8®, and Corn oil.

Another embodiment comprises cyclosporin A, Chemax E 200 ML®, and Corn oil.

Another embodiment comprises cyclosporin A, Chemax E 200 MO®, and Corn oil.

Another embodiment comprises cyclosporin A, Chemax E 400 ML®, and Corn oil.

Another embodiment comprises cyclosporin A, Chemax E 400 MO®, and Corn oil.

Another embodiment comprises cyclosporin A, Chemax E 600 ML®, and Corn oil.

Another embodiment comprises cyclosporin A, Chemax E 600 MO®, and Corn oil.

Another embodiment comprises cyclosporin A, Chemax E1000 MO®, and Corn oil.

Another embodiment comprises cyclosporin A, Chemax E1000 MS®, and Corn oil.

Another embodiment comprises cyclosporin A, Chemax E200 MS®, and Corn oil.

Another embodiment comprises cyclosporin A, Chemax E400 MS®, and Corn oil.

Another embodiment comprises cyclosporin A, Chemax E600 MS®, and Corn oil.

Another embodiment comprises cyclosporin A, Chemax HCO-16®, and Corn oil.

Another embodiment comprises cyclosporin A, Chemax HCO-200/50®, and Corn oil.

Another embodiment comprises cyclosporin A, Chemax HCO-25®, and Corn oil.

Another embodiment comprises cyclosporin A, Chemax HCO-5®, and Corn oil.

Another embodiment comprises cyclosporin A, Chemax TO-16®, and Corn oil.

Another embodiment comprises cyclosporin A, Chemax TO-8®, and Corn oil.

Another embodiment comprises cyclosporin A, Colalube 3409®, and Corn oil.

Another embodiment comprises cyclosporin A, Colalube 3411®, and Corn oil.

Another embodiment comprises cyclosporin A, Colalube 3413®, and Corn oil.

Another embodiment comprises cyclosporin A, Colalube 3414®, and Corn oil.

Another embodiment comprises cyclosporin A, Colalube 3415®, and Corn oil.

Another embodiment comprises cyclosporin A, Colalube 3416®, and Corn oil.

Another embodiment comprises cyclosporin A, Colalube 3417®, and Corn oil.

Another embodiment comprises cyclosporin A, Delonic NPE-100®, and Corn oil.

Another embodiment comprises cyclosporin A, Delonic NPE-30®, and Corn oil.

Another embodiment comprises cyclosporin A, Delonic NPE-40®, and Corn oil.

Another embodiment comprises cyclosporin A, Delonic OPE-10®, and Corn oil.

Another embodiment comprises cyclosporin A, Delonic OPE-12®, and Corn oil.

Another embodiment comprises cyclosporin A, Delonic OPE-30®, and Corn oil.

Another embodiment comprises cyclosporin A, Delonic OPE-40®, and Corn oil.

Another embodiment comprises cyclosporin A, Delonic OPE-5®, and Corn oil.

Another embodiment comprises cyclosporin A, Delonic OPE-7.5®, and Corn oil.

Another embodiment comprises cyclosporin A, DePeg 16-CO®, and Corn oil.

Another embodiment comprises cyclosporin A, DePeg 25-CO®, and Corn oil.

Another embodiment comprises cyclosporin A, DePeg 30-CO®, and Corn oil.

Another embodiment comprises cyclosporin A, DePeg 40-CO®, and Corn oil.

Another embodiment comprises cyclosporin A, DePeg 5-CO®, and Corn oil.

Another embodiment comprises cyclosporin A, DePeg 80-CO®, and Corn oil.

Another embodiment comprises cyclosporin A, DeSonic S-100®, and Corn oil.

Another embodiment comprises cyclosporin A, DeSonic S-114®, and Corn oil.

Another embodiment comprises cyclosporin A, DeSonic S-405®, and Corn oil.

Another embodiment comprises cyclosporin A, DeSonic S-45®, and Corn oil.

Another embodiment comprises cyclosporin A, DeSonic TDA-9®, and Corn oil.

Another embodiment comprises cyclosporin A, DeThox Acid O-14®, and Corn oil.

Another embodiment comprises cyclosporin A, DeThox Acid O-16®, and Corn oil.

Another embodiment comprises cyclosporin A, DeThox Acid O-9®, and Corn oil.

Another embodiment comprises cyclosporin A, DeThox Acid TO-14®, and Corn oil.

Another embodiment comprises cyclosporin A, DeThox Acid TO-16.5®, and Corn oil.

Another embodiment comprises cyclosporin A, DeThox Acid TO-8.5®, and Corn oil.

Another embodiment comprises cyclosporin A, Dur-Em 114®, and Corn oil.

Another embodiment comprises cyclosporin A, Dur-Em 117®, and Corn oil.

Another embodiment comprises cyclosporin A, Dur-Em 204®, and Corn oil.

Another embodiment comprises cyclosporin A, Dur-Em 207®, and Corn oil.

Another embodiment comprises cyclosporin A, Dur-Em 207E®, and Corn oil.

Another embodiment comprises cyclosporin A, Emerest 2704®, and Corn oil.

Another embodiment comprises cyclosporin A, Emerest 2712®, and Corn oil.

Another embodiment comprises cyclosporin A, Emerest 2715®, and Corn oil.

Another embodiment comprises cyclosporin A, Ethal EH-2®, and Corn oil.

Another embodiment comprises cyclosporin A, Ethal EH-5®, and Corn oil.

Another embodiment comprises cyclosporin A, Ethal OA-23®, and Corn oil.

Another embodiment comprises cyclosporin A, Ethosperse CA-20®, and Corn oil.

Another embodiment comprises cyclosporin A, Ethosperse LA-12®, and Corn oil.

Another embodiment comprises cyclosporin A, Ethosperse LA-23®, and Corn oil.

Another embodiment comprises cyclosporin A, Ethosperse LA-4®, and Corn oil.

Another embodiment comprises cyclosporin A, Ethox 3095®, and Corn oil.

Another embodiment comprises cyclosporin A, Ethox CO-16®, and Corn oil.

Another embodiment comprises cyclosporin A, Ethox CO-200®, and Corn oil.

Another embodiment comprises cyclosporin A, Ethox CO-25®, and Corn oil.

Another embodiment comprises cyciosporin A, Ethox CO-30®, and Corn oil.

Another embodiment comprises cyclosporin A, Ethox CO-36®, and Corn oil.

Another embodiment comprises cyclosporin A, Ethox CO-40®, and Corn oil.

Another embodiment comprises cyclosporin A, Ethox CO-5®, and Corn oil.

Another embodiment comprises cyclosporin A, Ethox DO-14®, and Corn oil.

Another embodiment comprises cyclosporin A, Ethox DO-9®, and Corn oil.

Another embodiment comprises cyclosporin A, Ethox MA-8®, and Corn oil.

Another embodiment comprises cyclosporin A, Ethox MI-14®, and Corn oil.

Another embodiment comprises cyclosporin A, Ethox ML-14®, and Corn oil.

Another embodiment comprises cyclosporin A, Ethox ML-5®, and Corn oil.

Another embodiment comprises cyclosporin A, Ethox MO-14®, and Corn oil.

Another embodiment comprises cyclosporin A, Ethox MO-9®, and Corn oil.

Another embodiment comprises cyclosporin A, Ethox MS-14®, and Corn oil.

Another embodiment comprises cyclosporin A, Ethox MS-23®, and Corn oil.

Another embodiment comprises cyclosporin A, Ethox MS-40®, and Corn oil.

Another embodiment comprises cyclosporin A, Ethox MS-8®, and Corn oil.

Another embodiment comprises cyclosporin A, Ethox TO-16®, and Corn oil.

Another embodiment comprises cyclosporin A, Ethox TO-8®, and Corn oil.

Another embodiment comprises cyclosporin A, Ethylan SN-120®, and Corn oil.

Another embodiment comprises cyclosporin A, Ethylan SN-70®, and Corn oil.

Another embodiment comprises cyclosporin A, Ethylan SN-90®, and Corn oil.

Another embodiment comprises cyclosporin A, Flexricin 13®, and Corn oil.

Another embodiment comprises cyclosporin A, Flexricin 15®, and Corn oil.

Another embodiment comprises cyclosporin A, Genapol 1261®, and Corn oil.

Another embodiment comprises cyclosporin A, Generol 122 E25®, and Corn oil.

Another embodiment comprises cyclosporin A, Glicepol 160®, and Corn oil.

Another embodiment comprises cyclosporin A, Glicepol 560®, and Corn oil.

Another embodiment comprises cyclosporin A, Glicepol GMS 20®, and Corn oil.

Another embodiment comprises cyclosporin A, Hetoxide BN-13®, and Corn oil.

Another embodiment comprises cyclosporin A, Hetoxide DNP-4®, and Corn oil.

Another embodiment comprises cyclosporin A, Hetoxide DNP-9.6®, and Corn oil.

Another embodiment comprises cyclosporin A, Hetoxide G-26®, and Corn oil.

Another embodiment comprises cyclosporin A, Hetoxide G-7®, and Corn oil.

Another embodiment comprises cyclosporin A, Hetoxide GT-20®, and Corn oil.

Another embodiment comprises cyclosporin A, Hetoxide HC-40®, and Corn oil.

Another embodiment comprises cyclosporin A, Hetoxol C-24®, and Corn oil.

Another embodiment comprises cyclosporin A, Hetoxol CA-10®, and Corn oil.

Another embodiment comprises cyclosporin A, Hetoxol CA-2®, and Corn oil.

Another embodiment comprises cyclosporin A, Hetoxol CA-20®, and Corn oil.

Another embodiment comprises cyclosporin A, Hetoxol CAWS®, and Corn oil.

Another embodiment comprises cyclosporin A, Hetoxol CD-3®, and Corn oil.

Another embodiment comprises cyclosporin A, Hetoxol CD-4®, and Corn oil.

Another embodiment comprises cyclosporin A, Hetoxol CS-15®, and Corn oil.

Another embodiment comprises cyclosporin A, Hetoxol CS-20®, and Corn oil.

Another embodiment comprises cyclosporin A, Hetoxol CS-20D®, and Corn oil.

Another embodiment comprises cyclosporin A, Hetoxol CS-25®, and Corn oil.

Another embodiment comprises cyclosporin A, Hetoxol CS-50®, and Corn oil.

Another embodiment comprises cyclosporin A, Hetoxol CS-9®, and Corn oil.

Another embodiment comprises cyclosporin A, Hetoxol CSA-15®, and Corn oil.

Another embodiment comprises cyclosporin A, Hetoxol D®, and Corn oil.

Another embodiment comprises cyclosporin A, Hetoxol G®, and Corn oil.

Another embodiment comprises cyclosporin A, Hetoxol J®, and Corn oil.

Another embodiment comprises cyclosporin A, Hetoxol L®, and Corn oil.

Another embodiment comprises cyclosporin A, Hetoxol L-23®, and Corn oil.

Another embodiment comprises cyclosporin A, Hetoxol L-4®, and Corn oil.

Another embodiment comprises cyclosporin A, Hetoxol L-9®, and Corn oil.

Another embodiment comprises cyclosporin A, Hetoxol LS-9®, and Corn oil.

Another embodiment comprises cyclosporin A, Hetoxol M-3®, and Corn oil.

Another embodiment comprises cyclosporin A, Hetoxol OA-10 Special®, and Corn oil.

Another embodiment comprises cyclosporin A, Hetoxol OA-20 Special®, and Corn oil.

Another embodiment comprises cyclosporin A, Hetoxol OA-3 Special®, and Corn oil.

Another embodiment comprises cyclosporin A, Hetoxol OA-5 Special®, and Corn oil.

Another embodiment comprises cyclosporin A, Hetoxol OL-10®, and Corn oil.

Another embodiment comprises cyclosporin A, Hetoxol OL-2®, and Corn oil.

Another embodiment comprises cyclosporin A, Hetoxol OL-23®, and Corn oil.

Another embodiment comprises cyclosporin A, Hetoxol OL-4®, and Corn oil.

Another embodiment comprises cyclosporin A, Hetoxol OL-40®, and Corn oil.

Another embodiment comprises cyclosporin A, Hetoxol OL-5®, and Corn oil.

Another embodiment comprises cyclosporin A, Hetoxol PLA®, and Corn oil.

Another embodiment comprises cyclosporin A, Hetoxol SP-15®, and Corn oil.

Another embodiment comprises cyclosporin A, Hetoxol STA-10®, and Corn oil.

Another embodiment comprises cyclosporin A, Hetoxol STA-2®, and Corn oil.

Another embodiment comprises cyclosporin A, Hetoxol STA-30®, and Corn oil.

Another embodiment comprises cyclosporin A, Hetoxol TD-12®, and Corn oil.

Another embodiment comprises cyclosporin A, Hetoxol TD-18®, and Corn oil.

Another embodiment comprises cyclosporin A, Hetoxol TD-3®, and Corn oil.

Another embodiment comprises cyclosporin A, Hetoxol TD-6®, and Corn oil.

Another embodiment comprises cyclosporin A, Hetoxol TD-9®, and Corn oil.

Another embodiment comprises cyclosporin A, Hetoxol TDEP-15®, and Corn oil.

Another embodiment comprises cyclosporin A, Hetoxol TDEP-63®, and Corn oil.

Another embodiment comprises cyclosporin A, Iconol 24-12®, and Corn oil.

Another embodiment comprises cyclosporin A, Iconol 24-23®, and Corn oil.

Another embodiment comprises cyclosporin A, Iconol 24-4®, and Corn oil.

Another embodiment comprises cyclosporin A, Iconol DA-4®, and Corn oil.

Another embodiment comprises cyclosporin A, Iconol DA-6®, and Corn oil.

Another embodiment comprises cyclosporin A, Iconol TDA-10®, and Corn oil.

Another embodiment comprises cyclosporin A, Iconol TDA-3®, and Corn oil.

Another embodiment comprises cyclosporin A, Iconol TDA-6®, and Corn oil.

Another embodiment comprises cyclosporin A, lconol TDA-8-90%®, and Corn oil.

Another embodiment comprises cyclosporin A, Iconol TDA-9®, and Corn oil.

Another embodiment comprises cyclosporin A, Igepal CA-210®, and Corn oil.

Another embodiment comprises cyclosporin A, Igepal CA-407®, and Corn oil.

Another embodiment comprises cyclosporin A, Igepal CA-420®, and Corn oil.

Another embodiment comprises cyclosporin A, Igepal CA-520®, Ernd Corn oil.

Another embodiment comprises cyclosporin A, Igepal CA-620®, and Corn oil.

Another embodiment comprises cyclosporin A, Igepal CA-630®, and Corn oil.

Another embodiment comprises cyclosporin A, Igepal CA-720®, and Corn oil.

Another embodiment comprises cyclosporin A, Igepal CA-877®, and Corn oil.

Another embodiment comprises cyclosporin A, Igepal CA-887®, and Corn oil.

Another embodiment comprises cyclosporin A, Igepal CA-897®, and Corn oil.

Another embodiment comprises cyclosporin A, Igepal CO-210®, and Corn oil.

Another embodiment comprises cyclosporin A, Igepal CO-430®, and Corn oil.

Another embodiment comprises cyclosporin A, Igepal CO-520®, and Corn oil.

Another embodiment comprises cyclosporin A, Igepal CO-530®, and Corn oil.

Another embodiment comprises cyclosporin A, Igepal CO-610®, and Corn oil.

Another embodiment comprises cyclosporin A, Igepal CO-630®, and Corn oil.

Another embodiment comprises cyclosporin A, Igepal CO-630 Special®, and Corn oil.

Another embodiment comprises cyclosporin A, Igepal CO-660®, and Corn oil.

Another embodiment comprises cyclosporin A, Igepal CO-710®, and Corn oil.

Another embodiment comprises cyclosporin A, Igepal CO-720®, and Corn oil.

Another embodiment comprises cyclosporin A, Igepal CO-730®, and Corn oil.

Another embodiment comprises cyclosporin A, Igepal CO-738®, and Corn oil.

Another embodiment comprises cyclosporin A, Igepal CO-850®, and Corn oil.

Another embodiment comprises cyclosporin A, Igepal CO-880®, and Corn oil.

Another embodiment comprises cyclosporin A, Igepal CO-887®, and Corn oil.

Another embodiment comprises cyclosporin A, Igepal CO-890®, and Corn oil.

Another embodiment comprises cyclosporin A, Igepal CO-897®, and Corn oil.

Another embodiment comprises cyclosporin A, Igepal CO-970®, and Corn oil.

Another embodiment comprises cyclosporin A, Igepal CO-977®, and Corn oil.

Another embodiment comprises cyclosporin A, Igepal CO-987®, and Corn oil.

Another embodiment comprises cyclosporin A, Igepal CO-990 FLK®, and Corn oil.

Another embodiment comprises cyclosporin A, Igepal CO-997®, and Corn oil.

Another embodiment comprises cyclosporin A, Igepal DM-430®, and Corn oil.

Another embodiment comprises cyclosporin A, Igepal DM-530®, and Corn oil.

Another embodiment comprises cyclosporin A, Igepal DM-710®, and Corn oil.

Another embodiment comprises cyclosporin A, Igepal DM-730®, and Corn oil.

Another embodiment comprises cyclosporin A, Igepal DM-880®, and Corn oil.

Another embodiment comprises cyclosporin A, Igepal DM-970 FLK®, and Corn oil.

Another embodiment comprises cyclosporin A, Igepal OD-410®, and Corn oil.

Another embodiment comprises cyclosporin A, Igepal RC-520®, and Corn oil.

Another embodiment comprises cyclosporin A, Igepal RC-620®, and Corn oil.

Another embodiment comprises cyclosporin A, Igepal RC-630®, and Corn oil.

Another embodiment comprises cyclosporin A, Lexemul CS-20®, and Corn oil.

Another embodiment comprises cyclosporin A, Lipo GMS 450®, and Corn oil.

Another embodiment comprises cyclosporin A, Lipo Polyglycol 1000®, and Corn oil.

Another embodiment comprises cyclosporin A, Lipo Polyglycol 200®, and Corn oil.

Another embodiment comprises cyclosporin A, Lipo Polyglycol 300®, and Corn oil.

Another embodiment comprises cyclosporin A, Lipo Polyglycol 3350®, and Corn oil.

Another embodiment comprises cyclosporin A, Lipo Polyglycol 40®, and Corn oil.

Another embodiment comprises cyclosporin A, Lipo Polyglycol 60®, and Corn oil.

Another embodiment comprises cyclosporin A, Lipocol C-10®, and Corn oil.

Another embodiment comprises cyclosporin A, Lipocol C-2®, and Corn oil.

Another embodiment comprises cyclosporin A, Lipocol C-20®, and Corn oil.

Another embodiment comprises cyclosporin A, Lipocol L-12®, and Corn oil.

Another embodiment comprises cyclosporin A, Lipocol L-23®, and Corn oil.

Another embodiment comprises cyclosporin A, Lipocol L-4®, and Corn oil.

Another embodiment comprises cyclosporin A, Lipocol O-10®, and Corn oil.

Another embodiment comprises cyclosporin A, Lipocol O-2®, and Corn oil.

Another embodiment comprises cyclosporin A, Lipocol O-20®, and Corn oil.

Another embodiment comprises cyclosporin A, Lipocol O-5®, and Corn oil.

Another embodiment comprises cyclosporin A, Lipocol S-10®, and Corn oil.

Another embodiment comprises cyclosporin A, Lipocol S-2®, and Corn oil.

Another embodiment comprises cyclosporin A, Lipocol S-20®, and Corn oil.

Another embodiment comprises cyclosporin A, Lipocol SC-15®, and Corn oil.

Another embodiment comprises cyclosporin A, Lipocol SC-20®, and Corn oil.

Another embodiment comprises cyclosporin A, Lipocol SC-4®, and Corn oil.

Another embodiment comprises cyclosporin A, Lipocol TD-12®, and Corn oil.

Another embodiment comprises cyclosporin A, Lumulse 40-O K®, and Corn oil.

Another embodiment comprises cyclosporin A, Lumulse 42-O K®, and Corn oil.

Another embodiment comprises cyclosporin A, Lumulse GMT-40®, and Corn oil.

Another embodiment comprises cyclosporin A, Lumulse PEG 1450®, and Corn oil.

Another embodiment comprises cyclosporin A, Lumulse PEG 1450 NF®, and Corn oil.

Another embodiment comprises cyclosporin A, Lumulse PEG 8000®, and Corn oil.

Another embodiment comprises cyclosporin A, Lumulse PGO®, and Corn oil.

Another embodiment comprises cyclosporin A, Lumulse POE (2) Oleyl Amine®, and Corn oil.

Another embodiment comprises cyclosporin A, Magrabar PGO-104®, and Corn oil.

Another embodiment comprises cyclosporin A, Mapeg 200 ML®, and Corn oil.

Another embodiment comprises cyclosporin A, Mapeg 400 MO®, and Corn oil.

Another embodiment comprises cyclosporin A, Mapeg 400 MOT®, and Corn oil.

Another embodiment comprises cyclosporin A, Mapeg 400DO®, and Corn oil.

Another embodiment comprises cyclosporin A, Mapeg 400DOT®, and Corn oil.

Another embodiment comprises cyclosporin A, Mapeg 600DOT®, and Corn oil.

Another embodiment comprises cyclosporin A, Marlox B 24/80®, and Corn oil.

Another embodiment comprises cyclosporin A, Marlox FK 86®, and Corn oil.

Another embodiment comprises cyclosporin A, Marlox MO 154®, and Corn oil.

Another embodiment comprises cyclosporin A, Mazox CDA CG®, and Corn oil.

Another embodiment comprises cyclosporin A, Monoglycerides Glyceryl Monestearate®, and Corn oil.

Another embodiment comprises cyclosporin A, Myrj 45®, and Corn oil.

Another embodiment comprises cyclosporin A, Myrj 52®, and Corn oil.

Another embodiment comprises cyclosporin A, Myrj 52S®, and Corn oil.

Another embodiment comprises cyclosporin A, Myrj 53®, and Corn oil.

Another embodiment comprises cyclosporin A, Myrj 59®, and Corn oil.

Another embodiment comprises cyclosporin A, Norfox NP-12®, and Corn oil.

Another embodiment comprises cyclosporin A, Norfox NP-4®, and Corn oil.

Another embodiment comprises cyclosporin A, Norfox NP-6®, and Corn oil.

Another embodiment comprises cyclosporin A, Norfox NP-887®, and Corn oil.

Another embodiment comprises cyclosporin A, Norfox NP-9®, and Corn oil.

Another embodiment comprises cyclosporin A, Norfox NP-977®, and Corn oil.

Another embodiment comprises cyclosporin A, Norfox OP-100®, and Corn oil.

Another embodiment comprises cyclosporin A, Norfox OP-102®, and Corn oil.

Another embodiment comprises cyclosporin A, Norfox OP-114®, and Corn oil.

Another embodiment comprises cyclosporin A, Norfox OP-45®, and Corn oil.

Another embodiment comprises cyclosporin A, Pegosperse 100-L®, and Corn oil.

Another embodiment comprises cyclosporin A, Pegosperse 100-O®, and Corn oil.

Another embodiment comprises cyclosporin A, Pegosperse 100-S®, and Corn oil.

Another embodiment comprises cyclosporin A, Pegosperse 1500-MS®, and Corn oil.

Another embodiment comprises cyclosporin A, Pegosperse 1750-MS®, and Corn oil.

Another embodiment comprises cyclosporin A, Pegosperse 200 DL®, and Corn oil.

Another embodiment comprises cyclosporin A, Pegosperse 200-ML®, and Corn oil.

Another embodiment comprises cyclosporin A, Pegosperse 400-DL®, and Corn oil.

Another embodiment comprises cyclosporin A, Pegosperse 400-DO®, and Corn oil.

Another embodiment comprises cyclosporin A, Pegosperse 400-DS®, and Corn oil.

Another embodiment comprises cyclosporin A, Pegosperse 400-ML®, and Corn oil.

Another embodiment comprises cyclosporin A, Pegosperse 400-MO®, and Corn oil.

Another embodiment comprises cyclosporin A, Pegosperse 400-MOT®, and Corn oil.

Another embodiment comprises cyclosporin A, Pegosperse 400-MS®, and Corn oil.

Another embodiment comprises cyclosporin A, Pegosperse 50-DS®, and Corn oil.

Another embodiment comprises cyclosporin A, Pegosperse 50-MS®, and Corn oil.

Another embodiment comprises cyclosporin A, Pegosperse 600-ML®, and Corn oil.

Another embodiment comprises cyclosporin A, Pegosperse 600-MS®, and Corn oil.

Another embodiment comprises cyclosporin A, Plurafac A-38®, and Corn oil.

Another embodiment comprises cyclosporin A, Plurafac B-25-5®, and Corn oil.

Another embodiment comprises cyclosporin A, Plurafac B-26®, and Corn oil.

Another embodiment comprises cyclosporin A, Plurafac D-25®, and Corn oil.

Another embodiment comprises cyclosporin A, Plurafac LF 131®, and Corn oil.

Another embodiment comprises cyclosporin A, Plurafac LF 4030®, and Corn oil.

Another embodiment comprises cyclosporin A, Plurafac LF 7000®, and Corn oil.

Another embodiment comprises cyclosporin A, Plurafac RA-20®, and Corn oil.

Another embodiment comprises cyclosporin A, Plurafac RA-30®, and Corn oil.

Another embodiment comprises cyclosporin A, Plurafac RA-40®, and Corn oil.

Another embodiment comprises cyclosporin A, Pluronic 10R5®, and Corn oil.

Another embodiment comprises cyclosporin A, Pluronic 17R2®, and Corn oil.

Another embodiment comprises cyclosporin A, Pluronic 17R4®, and Corn oil.

Another embodiment comprises cyclosporin A, Pluronic 25R2®, and Corn oil.

Another embodiment comprises cyclosporin A, Pluronic 25R4®, and Corn oil.

Another embodiment comprises cyclosporin A, Pluronic 31R1®, and Corn oil.

Another embodiment comprises cyclosporin A, Pluronic F108®, and Corn oil.

Another embodiment comprises cyclosporin A, Pluronic F127®, and Corn oil.

Another embodiment comprises cyclosporin A, Pluronic F38®, and Corn oil.

Another embodiment comprises cyclosporin A, Pluronic F68®, and Corn oil.

Another embodiment comprises cyclosporin A, Pluronic F68LF®, and Corn oil.

Another embodiment comprises cyclosporin A, Pluronic F77®, and Corn oil.

Another embodiment comprises cyclosporin A, Pluronic F87®, and Corn oil.

Another embodiment comprises cyclosporin A, Pluronic F88®, and Corn oil.

Another embodiment comprises cyclosporin A, Pluronic F98®, and Corn oil.

Another embodiment comprises cyclosporin A, Pluronic L10®, and Corn oil.

Another embodiment comprises cyclosporin A, Pluronic L101®, and Corn oil.

Another embodiment comprises cyclosporin A, Pluronic L121®, and Corn oil.

Another embodiment comprises cyclosporin A, Pluronic L31®, and Corn oil.

Another embodiment comprises cyclosporin A, Pluronic L35®, and Corn oil.

Another embodiment comprises cyclosporin A, Pluronic L43®, and Corn oil.

Another embodiment comprises cyclosporin A, Pluronic L44®, and Corn oil.

Another embodiment comprises cyclosporin A, Pluronic L61®, and Corn oil.

Another embodiment comprises cyclosporin A, Pluronic L62®, and Corn oil.

Another embodiment comprises cyclosporin A, Pluronic L62D®, and Corn oil.

Another embodiment comprises cyclosporin A, Pluronic L62LF®, and Corn oil.

Another embodiment comprises cyclosporin A, Pluronic L64®, and Corn oil.

Another embodiment comprises cyclosporin A, Pluronic L81®, and Corn oil.

Another embodiment comprises cyclosporin A, Pluronic L92®, and Corn oil.

Another embodiment comprises cyclosporin A, Pluronic P103®, and Corn oil.

Another embodiment comprises cyclosporin A, Pluronic P104®, and Corn oil.

Another embodiment comprises cyclosporin A, Pluronic P105®, and Corn oil.

Another embodiment comprises cyclosporin A, Pluronic P123®, and Corn oil.

Another embodiment comprises cyclosporin A, Pluronic P65®, and Corn oil.

Another embodiment comprises cyclosporin A, Pluronic P84®, and Corn oil.

Another embodiment comprises cyclosporin A, Pluronic P85®, and Corn oil.

Another embodiment comprises cyclosporin A, Polyaldo 10-2-P®, and Corn oil.

Another embodiment comprises cyclosporin A, Polyaldo DGDO®, and Corn oil.

Another embodiment comprises cyclosporin A, Polyaldo HGDS®, and Corn oil.

Another embodiment comprises cyclosporin A, Polyaldo TGMS®, and Corn oil.

Another embodiment comprises cyclosporin A, Protegin V®, and Corn oil.

Another embodiment comprises cyclosporin A, Protegin W®, and Corn oil.

Another embodiment comprises cyclosporin A, Protegin WX®, and Corn oil.

Another embodiment comprises cyclosporin A, Protegin XV®, and Corn oil.

Another embodiment comprises cyclosporin A, Rhodasurf BC-420®, and Corn oil.

Another embodiment comprises cyclosporin A, Rhodasurf BC-610®, and Corn oil.

Another embodiment comprises cyclosporin A, Rhodasurf BC-630®, and Corn oil.

Another embodiment comprises cyclosporin A, Rhodasurf BC-720®, and Corn oil.

Another embodiment comprises cyclosporin A, Rhodasurf BC-840®, and Corn oil.

Another embodiment comprises cyclosporin A, Rhodasurf L 1®, and Corn oil.

Another embodiment comprises cyclosporin A, Rhodasurf L 12®, and Corn oil.

Another embodiment comprises cyclosporin A, Rhodasurf L 2®, and Corn oil.

Another embodiment comprises cyclosporin A, Rhodasurf L 20®, and Corn oil.

Another embodiment comprises cyclosporin A, Rhodasurf L 3®, and Corn oil.

Another embodiment comprises cyclosporin A, Rhodasurf L 30®, and Corn oil.

Another embodiment comprises cyclosporin A, Rhodasurf L 4®, and Corn oil.

Another embodiment comprises cyclosporin A, Rhodasurf L 50®, and Corn oil.

Another embodiment comprises cyclosporin A, Rhodasurf L 7/90®, and Corn oil.

Another embodiment comprises cyclosporin A, Rhodasurf LA-3®, and Corn oil.

Another embodiment comprises cyclosporin A, Rhodasurf LA-7®, and Corn oil.

Another embodiment comprises cyclosporin A, Rhodasurf LAN-23®, and Corn oil.

Another embodiment comprises cyclosporin A, Rhodasurf ON-870®, and Corn oil.

Another embodiment comprises cyclosporin A, Rhodasurf ON-877®, and Corn oil.

Another embodiment comprises cyclosporin A, Rhodasurf TDA-8/5®, and Corn oil.

Another embodiment comprises cyclosporin A, Ritapro 100®, and Corn oil.

Another embodiment comprises cyclosporin A, Ritapro 165®, and Corn oil.

Another embodiment comprises cyclosporin A, Ritapro 200®, and Corn oil.

Another embodiment comprises cyclosporin A, Ritapro 300®, and Corn oil.

Another embodiment comprises cyclosporin A, Ritoleth 10®, and Corn oil.

Another embodiment comprises cyclosporin A, Ritoleth 2®, and Corn oil.

Another embodiment comprises cyclosporin A, Ritoleth 20®, and Corn oil.

Another embodiment comprises cyclosporin A, Ritoleth 5®, and Corn oil.

Another embodiment comprises cyclosporin A, Santone 10-10-0®, and Corn oil.

Another embodiment comprises cyclosporin A, Santone 3-1-S XTR®, and Corn oil.

Another embodiment comprises cyclosporin A, Santone 3-1-SH®, and Corn oil.

Another embodiment comprises cyclosporin A, Santone 8-1-O®, and Corn oil.

Another embodiment comprises cyclosporin A, Solulan 16®, and Corn oil.

Another embodiment comprises cyclosporin A, Solulan 75®, and Corn oil.

Another embodiment comprises cyclosporin A, Solulan C-24®, and Corn oil.

Another embodiment comprises cyclosporin A, Solulan L-575®, and Corn oil.

Another embodiment comprises cyclosporin A, Standamox CAW®, and Corn oil.

Another embodiment comprises cyclosporin A, Surfine AZI-A®, and Corn oil.

Another embodiment comprises cyclosporin A, Surfonic CO-15®, and Corn oil.

Another embodiment comprises cyclosporin A, Surfonic CO-25®, and Corn oil.

Another embodiment comprises cyclosporin A, Surfonic CO-30®, and Corn oil.

Another embodiment comprises cyclosporin A, Surfonic CO-36®, and Corn oil.

Another embodiment comprises cyclosporin A, Surfonic CO-42®, and Corn oil.

Another embodiment comprises cyclosporin A, Surfonic E400-MO®, and Corn oil.

Another embodiment comprises cyclosporin A, Surfonic L12-3®, and Corn oil.

Another embodiment comprises cyclosporin A, Surfonic L12-6®, and Corn oil.

Another embodiment comprises cyclosporin A, Surfonic L12-8®, and Corn oil.

Another embodiment comprises cyclosporin A, Surfonic L24-12®, and Corn oil.

Another embodiment comprises cyclosporin A, Surfonic L24-2®, and Corn oil.

Another embodiment comprises cyclosporin A, Surfonic L24-22®, and Corn oil.

Another embodiment comprises cyclosporin A, Surfonic L24-3®, and Corn oil.

Another embodiment comprises cyclosporin A, Surfonic L24-4®, and Corn oil.

Another embodiment comprises cyclosporin A, Surfonic L24-5®, and Corn oil.

Another embodiment comprises cyclosporin A, Surfonic L24-7®, and Corn oil.

Another embodiment comprises cyclosporin A, Surfonic L24-7.1®, and Corn oil.

Another embodiment comprises cyclosporin A, Surfonic L24-9®, and Corn oil.

Another embodiment comprises cyclosporin A, Surfonic LF-17®, and Corn oil.

Another embodiment comprises cyclosporin A, Surfonic LF-18®, and Corn oil.

Another embodiment comprises cyclosporin A, Surfonic LF-37®, and Corn oil.

Another embodiment comprises cyclosporin A, Surfonic LF-41®, and Corn oil.

Another embodiment comprises cyclosporin A, Surfonic LF-42®, and Corn oil.

Another embodiment comprises cyclosporin A, Surfonic LF-47®, and Corn oil.

Another embodiment comprises cyclosporin A, Surfonic LF-68®, and Corn oil.

Another embodiment comprises cyclosporin A, Surfonic N-10®, and Corn oil.

Another embodiment comprises cyclosporin A, Surfonic N-100®, and Corn oil.

Another embodiment comprises cyclosporin A, Surfonic N-102®, and Corn oil.

Another embodiment comprises cyclosporin A, Surfonic N-120®, and Corn oil.

Another embodiment comprises cyclosporin A, Surfonic N-150®, and Corn oil.

Another embodiment comprises cyclosporin A, Surfonic N-200®, and Corn oil.

Another embodiment comprises cyclosporin A, Surfonic N-300®, and Corn oil.

Another embodiment comprises cyclosporin A, Surfonic N-31.5®, and Corn oil.

Another embodiment comprises cyclosporin A, Surfonic N-40®, and Corn oil.

Another embodiment comprises cyclosporin A, Surfonic N-400®, and Corn oil.

Another embodiment comprises cyclosporin A, Surfonic N-60®, and Corn oil.

Another embodiment comprises cyclosporin A, Surfonic N-85®, and Corn oil.

Another embodiment comprises cyclosporin A, Surfonic N-95®, and Corn oil.

Another embodiment comprises cyclosporin A, Surfonic NB-18®, and Corn oil.

Another embodiment comprises cyclosporin A, Surfonic NB-23®, and Corn oil.

Another embodiment comprises cyclosporin A, Surfonic NB-25®, and Corn oil.

Another embodiment comprises cyclosporin A, Surfonic NB-307®, and Corn oil.

Another embodiment comprises cyclosporin A, Surfonic NB-31®, and Corn oil.

Another embodiment comprises cyclosporin A, Surfonic NB-41®, and Corn oil.

Another embodiment comprises cyclosporin A, Surfonic NB-43®, and Corn oil.

Another embodiment comprises cyclosporin A, Surfonic OP-100®, and Corn oil.

Another embodiment comprises cyclosporin A, Surfonic OP-120®, and Corn oil.

Another embodiment comprises cyclosporin A, Surfonic OP-15®, and Corn oil.

Another embodiment comprises cyclosporin A, Surfonic OP-35®, and Corn oil.

Another embodiment comprises cyclosporin A, Surfonic OP-70®, and Corn oil.

Another embodiment comprises cyclosporin A, Surfonic P1®, and Corn oil.

Another embodiment comprises cyclosporin A, Surfonic P3®, and Corn oil.

Another embodiment comprises cyclosporin A, Surfonic P5®, and Corn oil.

Another embodiment comprises cyclosporin A, Surfonic P6®, and Corn oil.

Another embodiment comprises cyclosporin A, Surfonic POA-17R2®, and Corn oil.

Another embodiment comprises cyclosporin A, Surfonic POA-25R2®, and Corn oil.

Another embodiment comprises cyclosporin A, Surfonic TDA-11®, and Corn oil.

Another embodiment comprises cyclosporin A, Surfonic TDA-3B®, and Corn oil.

Another embodiment comprises cyclosporin A, Surfonic TDA-6®, and Corn oil.

Another embodiment comprises cyclosporin A, Surfonic TDA-6/88®, and Corn oil.

Another embodiment comprises cyclosporin A, Surfonic TDA-8®, and Corn oil.

Another embodiment comprises cyclosporin A, Surfonic TDA-8/90®, and Corn oil.

Another embodiment comprises cyclosporin A, Surfonic TDA-9®, and Corn oil.

Another embodiment comprises cyclosporin A, Surfonic TX-CCN®, and Corn oil.

Another embodiment comprises cyclosporin A, Tagat CH 40®, and Corn oil.

Another embodiment comprises cyclosporin A, Tagat CH 60®, and Corn oil.

Another embodiment comprises cyclosporin A, Tagat L 2®, and Corn oil.

Another embodiment comprises cyclosporin A, Tagat S®, and Corn oil.

Another embodiment comprises cyclosporin A, Tagat S 2®, and Corn oil.

Another embodiment comprises cyclosporin A, Tally 100 Plus®, and Corn oil.

Another embodiment comprises cyclosporin A, T-Det A106®, and Corn oil.

Another embodiment comprises cyclosporin A, T-Det A109®, and Corn oil.

Another embodiment comprises cyclosporin A, T-Det A136®, and Corn oil.

Another embodiment comprises cyclosporin A, T-Det A139®, and Corn oil.

Another embodiment comprises cyclosporin A, T-Det A2412®, and Corn oil.

Another embodiment comprises cyclosporin A, T-Det A243®, and Corn oil.

Another embodiment comprises cyclosporin A, T-Det A247®, and Corn oil.

Another embodiment comprises cyclosporin A, T-Det A249®, and Corn oil.

Another embodiment comprises cyclosporin A, T-Det A467®, and Corn oil.

Another embodiment comprises cyclosporin A, T-Det COE®, and Corn oil.

Another embodiment comprises cyclosporin A, T-Det DD-10®, and Corn oil.

Another embodiment comprises cyclosporin A, T-Det DD-5®, and Corn oil.

Another embodiment comprises cyclosporin A, T-Det DD-7®, and Corn oil.

Another embodiment comprises cyclosporin A, T-Det EPO-104®, and Corn oil.

Another embodiment comprises cyclosporin A, T-Det EPO-61®, and Corn oil.

Another embodiment comprises cyclosporin A, T-Det EPO-62®, and Corn oil.

Another embodiment comprises cyclosporin A, T-Det EPO-64®, and Corn oil.

Another embodiment comprises cyclosporin A, T-Det N-1.5®, and Corn oil.

Another embodiment comprises cyclosporin A, T-Det N-10.5®, and Corn oil.

Another embodiment comprises cyclosporin A, T-Det N-100®, and Corn oil.

Another embodiment comprises cyclosporin A, T-Det N-1007®, and Corn oil.

Another embodiment comprises cyclosporin A, T-Det N-12®, and Corn oil.

Another embodiment comprises cyclosporin A, T-Det N-14®, and Corn oil.

Another embodiment comprises cyclosporin A, T-Det N-20®, and Corn oil.

Another embodiment comprises cyclosporin A, T-Det N-30®, and Corn oil.

Another embodiment comprises cyclosporin A, T-Det N-307®, and Corn oil.

Another embodiment comprises cyclosporin A, T-Det N-4®, and Corn oil.

Another embodiment comprises cyclosporin A, T-Det N-40®, and Corn oil.

Another embodiment comprises cyclosporin A, T-Det N-407®, and Corn oil.

Another embodiment comprises cyclosporin A, T-Det N-50®, and Corn oil.

Another embodiment comprises cyclosporin A, T-Det N-507®, and Corn oil.

Another embodiment comprises cyclosporin A, T-Det N-6®, and Corn oil.

Another embodiment comprises cyclosporin A, T-Det N-70®, and Corn oil.

Another embodiment comprises cyclosporin A, T-Det N-8®, and Corn oil.

Another embodiment comprises cyclosporin A, T-Det N-9.5®, and Corn oil.

Another embodiment comprises cyclosporin A, T-Det O-12®, and Corn oil.

Another embodiment comprises cyclosporin A, T-Det O-165®, and Corn oil.

Another embodiment comprises cyclosporin A, T-Det O-307®, and Corn oil.

Another embodiment comprises cyclosporin A, T-Det O-40®, and Corn oil.

Another embodiment comprises cyclosporin A, T-Det O-407®, and Corn oil.

Another embodiment comprises cyclosporin A, T-Det O-6®, and Corn oil.

Another embodiment comprises cyclosporin A, T-Det O-8®, and Corn oil.

Another embodiment comprises cyclosporin A, T-Det O-9®, and Corn oil.

Another embodiment comprises cyclosporin A, Tegin®, and Corn oil.

Another embodiment comprises cyclosporin A, Tergitol 15-S-12®, and Corn oil.

Another embodiment comprises cyclosporin A, Tergitol 15-S-15®, and Corn oil.

Another embodiment comprises cyclosporin A, Tergitol 15-S-20®, and Corn oil.

Another embodiment comprises cyclosporin A, Tergitol 15-S-20 (80AQ)®, and Corn oil.

Another embodiment comprises cyclosporin A, Tergitol 15-S-3®, and Corn oil.

Another embodiment comprises cyclosporin A, Tergitol 15-S-30®, and Corn oil.

Another embodiment comprises cyclosporin A, Tergitol 15-S-40®, and Corn oil.

Another embodiment comprises cyclosporin A, Tergitol 15-S-40 (70AQ)®, and Corn oil.

Another embodiment comprises cyclosporin A, Tergitol 15-S-5®, and Corn oil.

Another embodiment comprises cyclosporin A, Tergitol 15-S-7®, and Corn oil.

Another embodiment comprises cyclosporin A, Tergitol 15-S-9®, and Corn oil.

Another embodiment comprises cyclosporin A, Tergitol L-101®, and Corn oil.

Another embodiment comprises cyclosporin A, Tergitol L-61®, and Corn oil.

Another embodiment comprises cyclosporin A, Tergitol L-62®, and Corn oil.

Another embodiment comprises cyclosporin A, Tergitol L-64®, and Corn oil.

Another embodiment comprises cyclosporin A, Tergitol L-81®, and Corn oil.

Another embodiment comprises cyclosporin A, Tergitol NP-10®, and Corn oil.

Another embodiment comprises cyclosporin A, Tergitol NP-11®, and Corn oil.

Another embodiment comprises cyclosporin A, Tergitol NP-12®, and Corn oil.

Another embodiment comprises cyclosporin A, Tergitol NP-13®, and Corn oil.

Another embodiment comprises cyclosporin A, Tergitol NP-15®, and Corn oil.

Another embodiment comprises cyclosporin A, Tergitol NP-30®, and Corn oil.

Another embodiment comprises cyclosporin A, Tergitol NP-30 (70 AQ)®, and Corn oil.

Another embodiment comprises cyclosporin A, Tergitol NP-4®, and Corn oil.

Another embodiment comprises cyclosporin A, Tergitol NP-40®, and Corn oil.

Another embodiment comprises cyclosporin A, Tergitol NP-40 (70 AQ)®, and Corn oil.

Another embodiment comprises cyclosporin A, Tergitol NP-50 (70 AQ)®, and Corn oil.

Another embodiment comprises cyclosporin A, Tergitol NP-55 (70 AQ)®, and Corn oil.

Another embodiment comprises cyclosporin A, Tergitol NP-6®, and Corn oil.

Another embodiment comprises cyclosporin A, Tergitol NP-7®, and Corn oil.

Another embodiment comprises cyclosporin A, Tergitol NP-70 (70 AQ)®, and Corn oil.

Another embodiment comprises cyclosporin A, Tergitol NP-8®, and Corn oil.

Another embodiment comprises cyclosporin A, Tergitol NP-9®, and Corn oil.

Another embodiment comprises cyclosporin A, Tergitol NP-9.5®, and Corn oil.

Another embodiment comprises cyclosporin A, Tergitol TMN-10®, and Corn oil.

Another embodiment comprises cyclosporin A, Tergitol TMN-3®, and Corn oil.

Another embodiment comprises cyclosporin A, Tergitol TMN-6®, and Corn oil.

Another embodiment comprises cyclosporin A, Tergitol XD®, and Corn oil.

Another embodiment comprises cyclosporin A, Tergitol XH®, and Corn oil.

Another embodiment comprises cyclosporin A, Tergitol XJ®, and Corn oil.

Another embodiment comprises cyclosporin A, Tetronic 1107®, and Corn oil.

Another embodiment comprises cyclosporin A, Tetronic 1307®, and Corn oil.

Another embodiment comprises cyclosporin A, Tetronic 150R1®, and Corn oil.

Another embodiment comprises cyclosporin A, Tetronic 304®, and Corn oil.

Another embodiment comprises cyclosporin A, Tetronic 701®, and Corn oil.

Another embodiment comprises cyclosporin A, Tetronic 901®, and Corn oil.

Another embodiment comprises cyclosporin A, Tetronic 904®, and Corn oil.

Another embodiment comprises cyclosporin A, Tetronic 908®, and Corn oil.

Another embodiment comprises cyclosporin A, Tetronic 90R4®, and Corn oil.

Another embodiment comprises cyclosporin A, Tomadol 1-3®, and Corn oil.

Another embodiment comprises cyclosporin A, Tomadol 1-5®, and Corn oil.

Another embodiment comprises cyclosporin A, Tomadol 1-7®, and Corn oil.

Another embodiment comprises cyclosporin A, Tomadol 23-1®, and Corn oil.

Another embodiment comprises cyclosporin A, Tomadol 23-3®, and Corn oil.

Another embodiment comprises cyclosporin A, Tomadol 23-5®, and Corn oil.

Another embodiment comprises cyclosporin A, Tomadol 23-6.5®, and Corn oil.

Another embodiment comprises cyclosporin A, Tomadol 25-12®, and Corn oil.

Another embodiment comprises cyclosporin A, Tomadol 25-3®, and Corn oil.

Another embodiment comprises cyclosporin A, Tomadol 25-7®, and Corn oil.

Another embodiment comprises cyclosporin A, Tomadol 25-9®, and Corn oil.

Another embodiment comprises cyclosporin A, Tomadol 45-13®, and Corn oil.

Another embodiment comprises cyclosporin A, Tomadol 45-2.5®, and Corn oil.

Another embodiment comprises cyclosporin A, Tomadol 45-7®, and Corn oil.

Another embodiment comprises cyclosporin A, Tomadol 91-2.5®, and Corn oil.

Another embodiment comprises cyclosporin A, Tomadol 91-6®, and Corn oil.

Another embodiment comprises cyclosporin A, Tomadol 91-8®, and Corn oil.

Another embodiment comprises cyclosporin A, Triton X-100®, and Corn oil.

Another embodiment comprises cyclosporin A, Triton X-102®, and Corn oil.

Another embodiment comprises cyclosporin A, Triton X-114®, and Corn oil.

Another embodiment comprises cyclosporin A, Triton X-15®, and Corn oil.

Another embodiment comprises cyclosporin A, Triton X-165 (70%)®, and Corn oil.

Another embodiment comprises cyclosporin A, Triton X-207®, and Corn oil.

Another embodiment comprises cyclosporin A, Triton X-305 (70%)®, and Corn oil.

Another embodiment comprises cyclosporin A, Triton X-35®, and Corn oil.

Another embodiment comprises cyclosporin A, Triton X-405 (70%)®, and Corn oil.

Another embodiment comprises cyclosporin A, Triton X-45®, and Corn oil.

Another embodiment comprises cyclosporin A, Triton X-705 (70%)®, and Corn oil.

Another embodiment comprises cyclosporin A, Volpo S-10®, and Corn oil.

Another embodiment comprises cyclosporin A, Volpo S-2®, and Corn oil.

Another embodiment comprises cyclosporin A, Volpo S-20®, and Corn oil.

Another embodiment comprises cyclosporin A, Volpo-10®, and Corn oil.

Another embodiment comprises cyclosporin A, Volpo-20®, and Corn oil.

Another embodiment comprises cyclosporin A, Volpo-3®, and Corn oil.

Another embodiment comprises cyclosporin A, Volpo-5®, and Corn oil.

Another embodiment comprises cyclosporin A, Abex 2515®, and Arachis oil.

Another embodiment comprises cyclosporin A, Abex 2525/40®, and Arachis oil.

Another embodiment comprises cyclosporin A, Abex 2535®, and Arachis oil.

Another embodiment comprises cyclosporin A, Abex 2545®, and Arachis oil.

Another embodiment comprises cyclosporin A, Acconon E®, and Arachis oil.

Another embodiment comprises cyclosporin A, Alfonic 1216CO-9®, and Arachis oil.

Another embodiment comprises cyclosporin A, Alfonic 1412-3®, and Arachis oil.

Another embodiment comprises cyclosporin A, Alfonic TDA-12®, and Arachis oil.

Another embodiment comprises cyclosporin A, Alfonic TDA-3®, and Arachis oil.

Another embodiment comprises cyclosporin A, Alfonic TDA-4®, and Arachis oil.

Another embodiment comprises cyclosporin A, Alfonic TDA-6®, and Arachis oil.

Another embodiment comprises cyclosporin A, Alfonic TDA-7®, and Arachis oil.

Another embodiment comprises cyclosporin A, Alfonic TDA-8®, and Arachis oil.

Another embodiment comprises cyclosporin A, Alfonic TDA-9®, and Arachis oil.

Another embodiment comprises cyclosporin A, Alkamuls 400-DC®, and Arachis oil.

Another embodiment comprises cyclosporin A, Alkamuls 400-MO/E®, and Arachis oil.

Another embodiment comprises cyclosporin A, Ameroxol OE-10®, and Arachis oil.

Another embodiment comprises cyclosporin A, Ameroxol OE-2®, and Arachis oil.

Another embodiment comprises cyclosporin A, Ameroxol OE-20®, and Arachis oil.

Another embodiment comprises cyclosporin A, Armix 176®, and Arachis oil.

Another embodiment comprises cyclosporin A, Armix 180-C®, and Arachis oil.

Another embodiment comprises cyclosporin A, Armix 183®, and Arachis oil.

Another embodiment comprises cyclosporin A, Brij 35®, and Arachis oil.

Another embodiment comprises cyclosporin A, Brij 35 Liquid®, and Arachis oil.

Another embodiment comprises cyclosporin A, Brij 35 SP®, and Arachis oil.

Another embodiment comprises cyclosporin A, Canasol MJ 2109®, and Arachis oil.

Another embodiment comprises cyclosporin A, Canasol MJ 52®, and Arachis oil.

Another embodiment comprises cyclosporin A, Canasol R 3600®, and Arachis oil.

Another embodiment comprises cyclosporin A, Canasol R 3603®, and Arachis oil.

Another embodiment comprises cyclosporin A, Canasol R 4000 H®, and Arachis oil.

Another embodiment comprises cyclosporin A, Cedepal CA-210®, and Arachis oil.

Another embodiment comprises cyclosporin A, Cedepal CA-520®, and Arachis oil.

Another embodiment comprises cyclosporin A, Cedepal CA-630®, and Arachis oil.

Another embodiment comprises cyclosporin A, Cedepal CA-720®, and Arachis oil.

Another embodiment comprises cyclosporin A, Cedepal CA-890®, and Arachis oil.

Another embodiment comprises cyclosporin A, Cedepal CA-897®, and Arachis oil.

Another embodiment comprises cyclosporin A, Cedepal CO-210®, and Arachis oil.

Another embodiment comprises cyclosporin A, Cedepal CO-430®, and Arachis oil.

Another embodiment comprises cyclosporin A, Cedepal CO-530®, and Arachis oil.

Another embodiment comprises cyclosporin A, Cedepal CO-630®, and Arachis oil.

Another embodiment comprises cyclosporin A, Cedepal CO-710®, and Arachis oil.

Another embodiment comprises cyclosporin A, Cedepal CO-730®, and Arachis oil.

Another embodiment comprises cyclosporin A, Cedepal CO-880®, and Arachis oil.

Another embodiment comprises cyclosporin A, Cedepal CO-887®, and Arachis oil.

Another embodiment comprises cyclosporin A, Cedepal CO-890®, and Arachis oil.

Another embodiment comprises cyclosporin A, Cedepal CO-897®, and Arachis oil.

Another embodiment comprises cyclosporin A, Cedepal CO-970®, and Arachis oil.

Another embodiment comprises cyclosporin A, Cedepal CO-977®, and Arachis oil.

Another embodiment comprises cyclosporin A, Cedepal CO-990®, and Arachis oil.

Another embodiment comprises cyclosporin A, Cedepal CO-997®, and Arachis oil.

Another embodiment comprises cyclosporin A, Chemal BP 261®, and Arachis oil.

Another embodiment comprises cyclosporin A, Chemal BP 262®, and Arachis oil.

Another embodiment comprises cyclosporin A, Chemal BP 3172®, and Arachis oil.

Another embodiment comprises cyclosporin A, Chemal BP 3174®, and Arachis oil.

Another embodiment comprises cyclosporin A, Chemal DA-4®, and Arachis oil.

Another embodiment comprises cyclosporin A, Chemal DA-6®, and Arachis oil.

Another embodiment comprises cyclosporin A, Chemal DA-9®, and Arachis oil.

Another embodiment comprises cyclosporin A, Chemal LA-23®, and Arachis oil.

Another embodiment comprises cyclosporin A, Chemal LA-4®, and Arachis oil.

Another embodiment comprises cyclosporin A, Chemal LA-9®, and Arachis oil.

Another embodiment comprises cyclosporin A, Chemal OA-20G®, and Arachis oil.

Another embodiment comprises cyclosporin A, Chemal OA-5®, and Arachis oil.

Another embodiment comprises cyclosporin A, Chemal OA-9®, and Arachis oil.

Another embodiment comprises cyclosporin A, Chemal TDA-12®, and Arachis oil.

Another embodiment comprises cyclosporin A, Chemal TDA-15®, and Arachis oil.

Another embodiment comprises cyclosporin A, Chemal TDA-3®, and Arachis oil.

Another embodiment comprises cyclosporin A, Chemal TDA-6®, and Arachis oil.

Another embodiment comprises cyclosporin A, Chemal TDA-9®, and Arachis oil.

Another embodiment comprises cyclosporin A, Chemax CO-16®, and Arachis oil.

Another embodiment comprises cyclosporin A, Chemax CO-200/50®, and Arachis oil.

Another embodiment comprises cyclosporin A, Chemax CO-25®, and Arachis oil.

Another embodiment comprises cyclosporin A, Chemax CO-30®, and Arachis oil.

Another embodiment comprises cyclosporin A, Chemax CO-36®, and Arachis oil.

Another embodiment comprises cyclosporin A, Chemax CO-40®, and Arachis oil.

Another embodiment comprises cyclosporin A, Chemax CO-5®, and Arachis oil.

Another embodiment comprises cyclosporin A, Chemax CO-80®, and Arachis oil.

Another embodiment comprises cyclosporin A, Chemax DNP-150/50®, and Arachis oil.

Another embodiment comprises cyclosporin A, Chemax DNP-18®, and Arachis oil.

Another embodiment comprises cyclosporin A, Chemax DNP-8®, and Arachis oil.

Another embodiment comprises cyclosporin A, Chemax E 200 ML®, and Arachis oil.

Another embodiment comprises cyclosporin A, Chemax E 200 MO®, and Arachis oil.

Another embodiment comprises cyclosporin A, Chemax E 400 ML®, and Arachis oil.

Another embodiment comprises cyclosporin A, Chemax E 400 MO®, and Arachis oil.

Another embodiment comprises cyclosporin A, Chemax E 600 ML®, and Arachis oil.

Another embodiment comprises cyclosporin A, Chemax E 600 MO®, and Arachis oil.

Another embodiment comprises cyclosporin A, Chemax E1000 MO®, and Arachis oil.

Another embodiment comprises cyclosporin A, Chemax E1000 MS®, and Arachis oil.

Another embodiment comprises cyclosporin A, Chemax E200 MS®, and Arachis oil.

Another embodiment comprises cyclosporin A, Chemax E400 MS®, and Arachis oil.

Another embodiment comprises cyclosporin A, Chemax E600 MS®, and Arachis oil.

Another embodiment comprises cyclosporin A, Chemax HCO-16®, and Arachis oil.

Another embodiment comprises cyclosporin A, Chemax HCO-200/50®, and Arachis oil.

Another embodiment comprises cyclosporin A, Chemax HCO-25®, and Arachis oil.

Another embodiment comprises cyclosporin A, Chemax HCO-5®, and Arachis oil.

Another embodiment comprises cyclosporin A, Chemax TO-16®, and Arachis oil.

Another embodiment comprises cyclosporin A, Chemax TO-8®, and Arachis oil.

Another embodiment comprises cyclosporin A, Colalube 3409®, and Arachis oil.

Another embodiment comprises cyclosporin A, Colalube 3411®, and Arachis oil.

Another embodiment comprises cyclosporin A, Colalube 3413®, and Arachis oil.

Another embodiment comprises cyclosporin A, Colalube 3414®, and Arachis oil.

Another embodiment comprises cyclosporin A, Colalube 3415®, and Arachis oil.

Another embodiment comprises cyclosporin A, Colalube 3416®, and Arachis oil.

Another embodiment comprises cyclosporin A, Colalube 3417®, and Arachis oil.

Another embodiment comprises cyclosporin A, Delonic NPE-10®, and Arachis oil.

Another embodiment comprises cyclosporin A, Delonic NPE-30®, and Arachis oil.

Another embodiment comprises cyclosporin A, Delonic NPE-40®, and Arachis oil.

Another embodiment comprises cyclosporin A, Delonic OPE-10®, and Arachis oil.

Another embodiment comprises cyclosporin A, Delonic OPE-12®, and Arachis oil.

Another embodiment comprises cyclosporin A, Delonic OPE-30®, and Arachis oil.

Another embodiment comprises cyclosporin A, Delonic OPE-40®, and Arachis oil.

Another embodiment comprises cyclosporin A, Delonic OPE-5®, and Arachis oil.

Another embodiment comprises cyclosporin A, Delonic OPE-7.5®, and Arachis oil.

Another embodiment comprises cyclosporin A, DePeg 16-CO®, and Arachis oil.

Another embodiment comprises cyclosporin A, DePeg 25-CO®, and Arachis oil.

Another embodiment comprises cyclosporin A, DePeg 30-CO®, and Arachis oil.

Another embodiment comprises cyclosporin A, DePeg 40-CO®, and Arachis oil.

Another embodiment comprises cyclosporin A, DePeg 5-CO®, and Arachis oil.

Another embodiment comprises cyclosporin A, DePeg 80-CO®, and Arachis oil.

Another embodiment comprises cyclosporin A, DeSonic S-100®, and Arachis oil.

Another embodiment comprises cyclosporin A, DeSonic S-114®, and Arachis oil.

Another embodiment comprises cyclosporin A, DeSonic S-405®, and Arachis oil.

Another embodiment comprises cyclosporin A, DeSonic S-45®, and Arachis oil.

Another embodiment comprises cyclosporin A, DeSonic TDA-9®, and Arachis oil.

Another embodiment comprises cyclosporin A, DeThox Acid O-14®, and Arachis oil.

Another embodiment comprises cyclosporin A, DeThox Acid O-16®, and Arachis oil.

Another embodiment comprises cyclosporin A, DeThox Acid O-9®, and Arachis oil.

Another embodiment comprises cyclosporin A, DeThox Acid TO-14®, and Arachis oil.

Another embodiment comprises cyclosporin A, DeThox Acid TO-16.5®, and Arachis oil.

Another embodiment comprises cyclosporin A, DeThox Acid TO-8.5®, and Arachis oil.

Another embodiment comprises cyclosporin A, Dur-Em 114®, and Arachis oil.

Another embodiment comprises cyclosporin A, Dur-Em 117®, and Arachis oil.

Another embodiment comprises cyclosporin A, Dur-Em 204®, and Arachis oil.

Another embodiment comprises cyclosporin A, Dur-Em 207®, and Arachis oil.

Another embodiment comprises cyclosporin A, Dur-Em 207E®, and Arachis oil.

Another embodiment comprises cyclosporin A, Emerest 2704®, and Arachis oil.

Another embodiment comprises cyclosporin A, Emerest 2712®, and Arachis oil.

Another embodiment comprises cyclosporin A, Emerest 2715®, and Arachis oil.

Another embodiment comprises cyclosporin A, Ethal EH-2®, and Arachis oil.

Another embodiment comprises cyclosporin A, Ethal EH-5®, and Arachis oil.

Another embodiment comprises cyclosporin A, Ethal OA-23®, and Arachis oil.

Another embodiment comprises cyclosporin A, Ethosperse CA-20®, and Arachis oil.

Another embodiment comprises cyclosporin A, Ethosperse LA-12®, and Arachis oil.

Another embodiment comprises cyclosporin A, Ethosperse LA-23®, and Arachis oil.

Another embodiment comprises cyclosporin A, Ethosperse LA-4®, and Arachis oil.

Another embodiment comprises cyclosporin A, Ethox 3095®, and Arachis oil.

Another embodiment comprises cyclosporin A, Ethox CO-16®, and Arachis oil.

Another embodiment comprises cyclosporin A, Ethox CO-200®, and Arachis oil.

Another embodiment comprises cyclosporin A, Ethox CO-25®, and Arachis oil.

Another embodiment comprises cyclosporin A, Ethox CO-30®, and Arachis oil.

Another embodiment comprises cyclosporin A, Ethox CO-36®, and Arachis oil.

Another embodiment comprises cyclosporin A, Ethox CO-40®, and Arachis oil.

Another embodiment comprises cyclosporin A, Ethox CO-5®, and Arachis oil.

Another embodiment comprises cyclosporin A, Ethox DO-14®, and Arachis oil.

Another embodiment comprises cyclosporin A, Ethox DO-9®, and Arachis oil.

Another embodiment comprises cyclosporin A, Ethox MA-8®, and Arachis oil.

Another embodiment comprises cyclosporin A, Ethox MI-14®, and Arachis oil.

Another embodiment comprises cyclosporin A, Ethox ML-14®, and Arachis oil.

Another embodiment comprises cyclosporin A, Ethox ML-5®, and Arachis oil.

Another embodiment comprises cyclosporin A, Ethox MO-14®, and Arachis oil.

Another embodiment comprises cyclosporin A, Ethox MO-9®, and Arachis oil.

Another embodiment comprises cyclosporin A, Ethox MS-14®, and Arachis oil.

Another embodiment comprises cyclosporin A, Ethox MS-23®, and Arachis oil.

Another embodiment comprises cyclosporin A, Ethox MS-40®, and Arachis oil.

Another embodiment comprises cyclosporin A, Ethox MS-8®, and Arachis oil.

Another embodiment comprises cyclosporin A, Ethox TO-16®, and Arachis oil.

Another embodiment comprises cyclosporin A, Ethox TO-8®, and Arachis oil.

Another embodiment comprises cyclosporin A, Ethylan SN-120®, and Arachis oil.

Another embodiment comprises cyclosporin A, Ethylan SN-70®, and Arachis oil.

Another embodiment comprises cyclosporin A, Ethylan SN-90®, and Arachis oil.

Another embodiment comprises cyclosporin A, Flexricin 13®, and Arachis oil.

Another embodiment comprises cyclosporin A, Flexricin 15®, and Arachis oil.

Another embodiment comprises cyclosporin A, Genapol 1261®, and Arachis oil.

Another embodiment comprises cyclosporin A, Generol 122 E25®, and Arachis oil.

Another embodiment comprises cyclosporin A, Glicepol 160®, and Arachis oil.

Another embodiment comprises cyclosporin A, Glicepol 560®, and Arachis oil.

Another embodiment comprises cyclosporin A, Glicepol GMS 20®, and Arachis oil.

Another embodiment comprises cyclosporin A, Hetoxide BN-13®, and Arachis oil.

Another embodiment comprises cyclosporin A, Hetoxide DNP-4®, and Arachis oil.

Another embodiment comprises cyclosporin A, Hetoxide DNP-9.6®, and Arachis oil.

Another embodiment comprises cyclosporin A, Hetoxide G-26®, and Arachis oil.

Another embodiment comprises cyclosporin A, Hetoxide G-7®, and Arachis oil.

Another embodiment comprises cyclosporin A, Hetoxide GT-20®, and Arachis oil.

Another embodiment comprises cyclosporin A, Hetoxide HC-40®, and Arachis oil.

Another embodiment comprises cyclosporin A, Hetoxol C-24®, and Arachis oil.

Another embodiment comprises cyclosporin A, Hetoxol CA-10®, and Arachis oil.

Another embodiment comprises cyclosporin A, Hetoxol CA-2®, and Arachis oil.

Another embodiment comprises cyclosporin A, Hetoxol CA-20®, and Arachis oil.

Another embodiment comprises cyclosporin A, Hetoxol CAWS®, and Arachis oil.

Another embodiment comprises cyclosporin A, Hetoxol CD-3®, and Arachis oil.

Another embodiment comprises cyclosporin A, Hetoxol CD-4®, and Arachis oil.

Another embodiment comprises cyclosporin A, Hetoxol CS-15®, and Arachis oil.

Another embodiment comprises cyclosporin A, Hetoxol CS-20®, and Arachis oil.

Another embodiment comprises cyclosporin A, Hetoxol CS-20D®, and Arachis oil.

Another embodiment comprises cyclosporin A, Hetoxol CS-25®, and Arachis oil.

Another embodiment comprises cyclosporin A, Hetoxol CS-50®, and Arachis oil.

Another embodiment comprises cyclosporin A, Hetoxol CS-9®, and Arachis oil.

Another embodiment comprises cyclosporin A, Hetoxol CSA-15®, and Arachis oil.

Another embodiment comprises cyclosporin A, Hetoxol D®, and Arachis oil.

Another embodiment comprises cyclosporin A, Hetoxol G®, and Arachis oil.

Another embodiment comprises cyclosporin A, Hetoxol J®, and Arachis oil.

Another embodiment comprises cyclosporin A, Hetoxol L®, and Arachis oil.

Another embodiment comprises cyclosporin A, Hetoxol L-23®, and Arachis oil.

Another embodiment comprises cyclosporin A, Hetoxol L-4®, and Arachis oil.

Another embodiment comprises cyclosporin A, Hetoxol L-9®, and Arachis oil.

Another embodiment comprises cyclosporin A, Hetoxol LS-9®, and Arachis oil.

Another embodiment comprises cyclosporin A, Hetoxol M-3®, and Arachis oil.

Another embodiment comprises cyclosporin A, Hetoxol OA-10 Special®, and Arachis oil.

Another embodiment comprises cyclosporin A, Hetoxol OA-20 Special®, and Arachis oil.

Another embodiment comprises cyclosporin A, Hetoxol OA-3 Special®, and Arachis oil.

Another embodiment comprises cyclosporin A, Hetoxol OA-5 Special®, and Arachis oil.

Another embodiment comprises cyclosporin A, Hetoxol OL-10®, and Arachis oil.

Another embodiment comprises cyclosporin A, Hetoxol OL-2®, and Arachis oil.

Another embodiment comprises cyclosporin A, Hetoxol OL-23®, and Arachis oil.

Another embodiment comprises cyclosporin A, Hetoxol OL-4®, and Arachis oil.

Another embodiment comprises cyclosporin A, Hetoxol OL-40®, and Arachis oil.

Another embodiment comprises cyclosporin A, Hetoxol OL-5®, and Arachis oil.

Another embodiment comprises cyclosporin A, Hetoxol PLA®, and Arachis oil.

Another embodiment comprises cyclosporin A, Hetoxol SP-15®, and Arachis oil.

Another embodiment comprises cyclosporin A, Hetoxol STA-10®, and Arachis oil.

Another embodiment comprises cyclosporin A, Hetoxol STA-2®, and Arachis oil.

Another embodiment comprises cyclosporin A, Hetoxol STA-30®, and Arachis oil.

Another embodiment comprises cyclosporin A, Hetoxol TD-12®, and Arachis oil.

Another embodiment comprises cyclosporin A, Hetoxol TD-18®, and Arachis oil.

Another embodiment comprises cyclosporin A, Hetoxol TD-3®, and Arachis oil.

Another embodiment comprises cyclosporin A, Hetoxol TD-6®, and Arachis oil.

Another embodiment comprises cyclosporin A, Hetoxol TD-9®, and Arachis oil.

Another embodiment comprises cyclosporin A, Hetoxol TDEP-15®, and Arachis oil.

Another embodiment comprises cyclosporin A, HetoxQl TDEP-63®, and Arachis oil.

Another embodiment comprises cyclosporin A, Iconol 24-12®, and Arachis oil.

Another embodiment comprises cyclosporin A, Iconol 24-23®, and Arachis oil.

Another embodiment comprises cyclosporin A, Iconol 24-4®, and Arachis oil.

Another embodiment comprises cyclosporin A, Iconol DA-4®, and Arachis oil.

Another embodiment comprises cyclosporin A, Iconol DA-6®, and Arachis oil.

Another embodiment comprises cyclosporin A, Iconol TDA-10®, and Arachis oil.

Another embodiment comprises cyclosporin A, Iconol TDA-3®, and Arachis oil.

Another embodiment comprises cyclosporin A, Iconol TDA-6®, and Arachis oil.

Another embodiment comprises cyclosporin A, Iconol TDA-8-90%®, and Arachis oil.

Another embodiment comprises cyclosporin A, Iconol TDA-9®, and Arachis oil.

Another embodiment comprises cyclosporin A, Igepal CA-210®, and Arachis oil.

Another embodiment comprises cyclosporin A, Igepal CA-407®, and Arachis oil.

Another embodiment comprises cyclosporin A, Igepal CA-420®, and Arachis oil.

Another embodiment comprises cyclosporin A, Igepal CA-520®, and Arachis oil.

Another embodiment comprises cyclosporin A, Igepal CA-620®, and Arachis oil.

Another embodiment comprises cyclosporin A, Igepal CA-630®, and Arachis oil.

Another embodiment comprises cyclosporin A, Igepal CA-720®, and Arachis oil.

Another embodiment comprises cyclosporin A, Igepal CA-877®, and Arachis oil.

Another embodiment comprises cyclosporin A, Igepal CA-887®, and Arachis oil.

Another embodiment comprises cyclosporin A, Igepal CA-897®, and Arachis oil.

Another embodiment comprises cyclosporin A, Igepal CO-210®, and Arachis oil.

Another embodiment comprises cyclosporin A, Igepal CO-430®, and Arachis oil.

Another embodiment comprises cyclosporin A, Igepal CO-520®, and Arachis oil.

Another embodiment comprises cyclosporin A, Igepal CO-530®, and Arachis oil.

Another embodiment comprises cyclosporin A, Igepal CO-610®, and Arachis oil.

Another embodiment comprises cyclosporin A, Igepal CO-630®, and Arachis oil.

Another embodiment comprises cyclosporin A, Igepal CO-630 Special®, and Arachis oil.

Another embodiment comprises cyclosporin A, Igepal CO-660®, and Arachis oil.

Another embodiment comprises cyclosporin A, Igepal CO-710®, and Arachis oil.

Another embodiment comprises cyclosporin A, Igepal CO-720®, and Arachis oil.

Another embodiment comprises cyclosporin A, Igepal CO-730®, and Arachis oil.

Another embodiment comprises cyclosporin A, Igepal CO-738®, and Arachis oil.

Another embodiment comprises cyclosporin A, Igepal CO-850®, and Arachis oil.

Another embodiment comprises cyclosporin A, Igepal CO-880®, and Arachis oil.

Another embodiment comprises cyclosporin A, Igepal CO-887®, and Arachis oil.

Another embodiment comprises cyclosporin A, Igepal CO-890®, and Arachis oil.

Another embodiment comprises cyclosporin A, Igepal CO-897®, and Arachis oil.

Another embodiment comprises cyclosporin A, Igepal CO-970®, and Arachis oil.

Another embodiment comprises cyclosporin A, Igepal CO-977®, and Arachis oil.

Another embodiment comprises cyclosporin A, Igepal CO-987®, and Arachis oil.

Another embodiment comprises cyclosporin A, Igepal CO-990 FLK®, and Arachis oil.

Another embodiment comprises cyclosporin A, Igepal CO-997®, and Arachis oil.

Another embodiment comprises cyclosporin A, Igepal DM-430®, and Arachis oil.

Another embodiment comprises cyclosporin A, Igepal DM-530®, and Arachis oil.

Another embodiment comprises cyclosporin A, Igepal DM-710®, and Arachis oil.

Another embodiment comprises cyclosporin A, Igepal DM-730®, and Arachis oil.

Another embodiment comprises cyclosporin A, Igepal DM-880®, and Arachis oil.

Another embodiment comprises cyclosporin A, Igepal DM-970 FLK®, and Arachis oil.

Another embodiment comprises cyclosporin A, Igepal OD-410®, and Arachis oil.

Another embodiment comprises cyclosporin A, Igepal RC-520®, and Arachis oil.

Another embodiment comprises cyclosporin A, Igepal RC-620®, and Arachis oil.

Another embodiment comprises cyclosporin A, Igepal RC-630®, and Arachis oil.

Another embodiment comprises cyclosporin A, Lexemul CS-20®, and Arachis oil.

Another embodiment comprises cyclosporin A, Lipo GMS 450®, and Arachis oil.

Another embodiment comprises cyclosporin A, Lipo Polyglycol 1000®, and Arachis oil.

Another embodiment comprises cyclosporin A, Lipo Polyglycol 200®, and Arachis oil.

Another embodiment comprises cyclosporin A, Lipo Polyglycol 300®, and Arachis oil.

Another embodiment comprises cyclosporin A, Lipo Polyglycol 3350®, and Arachis oil.

Another embodiment comprises cyclosporin A, Lipo Polyglycol 400®, and Arachis oil.

Another embodiment comprises cyclosporin A, Lipo Polyglycol 600®, and Arachis oil.

Another embodiment comprises cyclosporin A, Lipocol C-10®, and Arachis oil.

Another embodiment comprises cyclosporin A, Lipocol C-2®, and Arachis oil.

Another embodiment comprises cyclosporin A, Lipocol C-20®, and Arachis oil.

Another embodiment comprises cyclosporin A, Lipocol L-12®, and Arachis oil.

Another embodiment comprises cyclosporin A, Lipocol L-23®, and Arachis oil.

Another embodiment comprises cyclosporin A, Lipocol L-4®, and Arachis oil.

Another embodiment comprises cyclosporin A, Lipocol O-10®, and Arachis oil.

Another embodiment comprises cyclosporin A, Lipocol O-2®, and Arachis oil.

Another embodiment comprises cyclosporin A, Lipocol O-20®, and Arachis oil.

Another embodiment comprises cyclosporin A, Lipocol O-5®, and Arachis oil.

Another embodiment comprises cyclosporin A, Lipocol S-10®, and Arachis oil.

Another embodiment comprises cyclosporin A, Lipocol S-2®, and Arachis oil.

Another embodiment comprises cyclosporin A, Lipocol S-20®, and Arachis oil.

Another embodiment comprises cyclosporin A, Lipocol SC-15®, and Arachis oil.

Another embodiment comprises cyclosporin A, Lipocol SC-20®, and Arachis oil.

Another embodiment comprises cyclosporin A, Lipocol SC-4®, and Arachis oil.

Another embodiment comprises cyclosporin A, Lipocol TD-12®, and Arachis oil.

Another embodiment comprises cyclosporin A, Lumulse 40-O K®, and Arachis oil.

Another embodiment comprises cyclosporin A, Lumulse 42-O K®, and Arachis oil.

Another embodiment comprises cyclosporin A, Lumulse GMT-40®, and Arachis oil.

Another embodiment comprises cyclosporin A, Lumulse PEG 1450®, and Arachis oil.

Another embodiment comprises cyclosporin A, Lumulse PEG 1450 NF®, and Arachis oil.

Another embodiment comprises cyclosporin A, Lumulse PEG 8000®, and Arachis oil.

Another embodiment comprises cyclosporin A, Lumulse PGO®, and Arachis oil.

Another embodiment comprises cyclosporin A, Lumulse POE (2) Oleyl Amine®, and Arachis oil.

Another embodiment comprises cyclosporin A, Magrabar PGO-104®, and Arachis oil.

Another embodiment comprises cyclosporin A, Mapeg 200 ML®, and Arachis oil.

Another embodiment comprises cyclosporin A, Mapeg 400 MO®, and Arachis oil.

Another embodiment comprises cyclosporin A, Mapeg 400 MOT®, and Arachis oil.

Another embodiment comprises cyclosporin A, Mapeg 400DO®, and Arachis oil.

Another embodiment comprises cyclosporin A, Mapeg 400DOT®, and Arachis oil.

Another embodiment comprises cyclosporin A, Mapeg 600DOT®, and Arachis oil.

Another embodiment comprises cyclosporin A, Marlox B 24/80®, and Arachis oil.

Another embodiment comprises cyclosporin A, Marlox FK 86®, and Arachis oil.

Another embodiment comprises cyclosporin A, Marlox MO 154®, and Arachis oil.

Another embodiment comprises cyclosporin A, Mazox CDA CG®, and Arachis oil.

Another embodiment comprises cyclosporin A, Monoglycerides Glyceryl Monestearate®, and Arachis oil.

Another embodiment comprises cyclosporin A, Myrj 45®, and Arachis oil.

Another embodiment comprises cyclosporin A, Myrj 52®, and Arachis oil.

Another embodiment comprises cyclosporin A, Myrj 52S®, and Arachis oil.

Another embodiment comprises cyclosporin A, Myrj 53®, and Arachis oil.

Another embodiment comprises cyclosporin A, Myrj 59®, and Arachis oil.

Another embodiment comprises cyclosporin A, Norfox NP-12®, and Arachis oil.

Another embodiment comprises cyclosporin A, Norfox NP-4®, and Arachis oil.

Another embodiment comprises cyclosporin A, Norfox NP-6®, and Arachis oil.

Another embodiment comprises cyclosporin A, Norfox NP-887®, and Arachis oil.

Another embodiment comprises cyclosporin A, Norfox NP-9®, and Arachis oil.

Another embodiment comprises cyclosporin A, Norfox NP-977®, and Arachis oil.

Another embodiment comprises cyclosporin A, Norfox OP-100®, and Arachis oil.

Another embodiment comprises cyclosporin A, Norfox OP-102®, and Arachis oil.

Another embodiment comprises cyclosporin A, Norfox OP-114®, and Arachis oil.

Another embodiment comprises cyclosporin A, Norfox OP-45®, and Arachis oil.

Another embodiment comprises cyclosporin A, Pegosperse 100-L®, and Arachis oil.

Another embodiment comprises cyclosporin A, Pegosperse 100-O®, and Arachis oil.

Another embodiment comprises cyclosporin A, Pegosperse 100-S®, and Arachis oil.

Another embodiment comprises cyclosporin A, Pegosperse 1500-MS®, and Arachis oil.

Another embodiment comprises cyclosporin A, Pegosperse 1750-MS®, and Arachis oil.

Another embodiment comprises cyclosporin A, Pegosperse 200 DL®, and Arachis oil.

Another embodiment comprises cyclosporin A, Pegosperse 200-ML®, and Arachis oil.

Another embodiment comprises cyclosporin A, Pegosperse 400-DL®, and Arachis oil.

Another embodiment comprises cyclosporin A, Pegosperse 400-DO®, and Arachis oil.

Another embodiment comprises cyclosporin A, Pegosperse 400-DS®, and Arachis oil.

Another embodiment comprises cyclosporin A, Pegosperse 400-ML®, and Arachis oil.

Another embodiment comprises cyclosporin A, Pegosperse 400-MO®, and Arachis oil.

Another embodiment comprises cyclosporin A, Pegosperse 400-MOT®, and Arachis oil.

Another embodiment comprises cyclosporin A, Pegosperse 400-MS®, and Arachis oil.

Another embodiment comprises cyclosporin A, Pegosperse 50-DS®, and Arachis oil.

Another embodiment comprises cyclosporin A, Pegosperse 50-MS®, and Arachis oil.

Another embodiment comprises cyclosporin A, Pegosperse 600-ML®, and Arachis oil.

Another embodiment comprises cyclosporin A, Pegosperse 600-MS®, and Arachis oil.

Another embodiment comprises cyclosporin A, Plurafac A-38®, and Arachis oil.

Another embodiment comprises cyclosporin A, Plurafac B-25-5®, and Arachis oil.

Another embodiment comprises cyclosporin A, Plurafac B-26®, and Arachis oil.

Another embodiment comprises cyclosporin A, Plurafac D-25®, and Arachis oil.

Another embodiment comprises cyclosporin A, Plurafac LF 131®, and Arachis oil.

Another embodiment comprises cyclosporin A, Plurafac LF 4030®, and Arachis oil.

Another embodiment comprises cyclosporin A, Plurafac LF 7000®, and Arachis oil.

Another embodiment comprises cyclosporin A, Plurafac RA-20®, and Arachis oil.

Another embodiment comprises cyclosporin A, Plurafac RA-30®, and Arachis oil.

Another embodiment comprises cyclosporin A, Plurafac RA-40®, and Arachis oil.

Another embodiment comprises cyclosporin A, Pluronic 10R®, and Arachis oil.

Another embodiment comprises cyclosporin A, Pluronic 17R2®, and Arachis oil.

Another embodiment comprises cyclosporin A, Pluronic 17R4®, and Arachis oil.

Another embodiment comprises cyclosporin A, Pluronic 25R2®, and Arachis oil.

Another embodiment comprises cyclosporin A, Pluronic 25R4®, and Arachis oil.

Another embodiment comprises cyclosporin A, Pluronic 31R1®, and Arachis oil.

Another embodiment comprises cyclosporin A, Pluronic F108®, and Arachis oil.

Another embodiment comprises cyclosporin A, Pluronic F127®, and Arachis oil.

Another embodiment comprises cyclosporin A, Pluronic F38®, and Arachis oil.

Another embodiment comprises cyclosporin A, Pluronic F68®, and Arachis oil.

Another embodiment comprises cyclosporin A, Pluronic F68LF®, and Arachis oil.

Another embodiment comprises cyclosporin A, Pluronic F77®, and Arachis oil.

Another embodiment comprises cyclosporin A, Pluronic F87®, and Arachis oil.

Another embodiment comprises cyclosporin A, Pluronic F88®, and Arachis oil.

Another embodiment comprises cyclosporin A, Pluronic F98®, and Arachis oil.

Another embodiment comprises cyclosporin A, Pluronic L10®, and Arachis oil.

Another embodiment comprises cyclosporin A, Pluronic L101®, and Arachis oil.

Another embodiment comprises cyclosporin A, Pluronic L121®, and Arachis oil.

Another embodiment comprises cyclosporin A, Pluronic L31®, and Arachis oil.

Another embodiment comprises cyclosporin A, Pluronic L35®, and Arachis oil.

Another embodiment comprises cyclosporin A, Pluronic L43®, and Arachis oil.

Another embodiment comprises cyclosporin A, Pluronic L44®, and Arachis oil.

Another embodiment comprises cyclosporin A, Pluronic L61®, and Arachis oil.

Another embodiment comprises cyclosporin A, Pluronic L62®, and Arachis oil.

Another embodiment comprises cyclosporin A, Pluronic L62D®, and Arachis oil.

Another embodiment comprises cyclosporin A, Pluronic L62LF®, and Arachis oil.

Another embodiment comprises cyclosporin A, Pluronic L64®, and Arachis oil.

Another embodiment comprises cyclosporin A, Pluronic L81®, and Arachis oil.

Another embodiment comprises cyclosporin A, Pluronic L92®, and Arachis oil.

Another embodiment comprises cyclosporin A, Pluronic P103®, and Arachis oil.

Another embodiment comprises cyclosporin A, Pluronic P104®, and Arachis oil.

Another embodiment comprises cyclosporin A, Pluronic P105®, and Arachis oil.

Another embodiment comprises cyclosporin A, Pluronic P123®, and Arachis oil.

Another embodiment comprises cyclosporin A, Pluronic P65®, and Arachis oil.

Another embodiment comprises cyclosporin A, Pluronic P84®, and Arachis oil.

Another embodiment comprises cyclosporin A, Pluronic P85®, and Arachis oil.

Another embodiment comprises cyclosporin A, Polyaldo 10-2-P®, and Arachis oil.

Another embodiment comprises cyclosporin A, Polyaldo DGDO®, and Arachis oil.

Another embodiment comprises cyclosporin A, Polyaldo HGDS®, and Arachis oil.

Another embodiment comprises cyclosporin A, Polyaldo TGMS®, and Arachis oil.

Another embodiment comprises cyclosporin A, Protegin V®, and Arachis oil.

Another embodiment comprises cyclosporin A, Protegin W®, and Arachis oil.

Another embodiment comprises cyclosporin A, Protegin WX®, and Arachis oil.

Another embodiment comprises cyclosporin A, Protegin XV®, and Arachis oil.

Another embodiment comprises cyclosporin A, Rhodasurf BC-420®, and Arachis oil.

Another embodiment comprises cyclosporin A, Rhodasurf BC-610®, and Arachis oil.

Another embodiment comprises cyclosporin A, Rhodasurf BC-630®, and Arachis oil.

Another embodiment comprises cyclosporin A, Rhodasurf BC-720®, and Arachis oil.

Another embodiment comprises cyclosporin A, Rhodasurf BC-840®, and Arachis oil.

Another embodiment comprises cyclosporin A, Rhodasurf L 1®, and Arachis oil.

Another embodiment comprises cyclosporin A, Rhodasurf L 12®, and Arachis oil.

Another embodiment comprises cyclosporin A, Rhodasurf L 2®, and Arachis oil.

Another embodiment comprises cyclosporin A, Rhodasurf L 20®, and Arachis oil.

Another embodiment comprises cyclosporin A, Rhodasurf L 3®, and Arachis oil.

Another embodiment comprises cyclosporin A, Rhodasurf L 30®, and Arachis oil.

Another embodiment comprises cyclosporin A, Rhodasurf L 4®, and Arachis oil.

Another embodiment comprises cyclosporin A, Rhodasurf L 50®, and Arachis oil.

Another embodiment comprises cyclosporin A, Rhodasurf L 7/90®, and Arachis oil.

Another embodiment comprises cyclosporin A, Rhodasurf LA-3®, and Arachis oil.

Another embodiment comprises cyclosporin A, Rhodasurf LA-7®, and Arachis oil.

Another embodiment comprises cyclosporin A, Rhodasurf LAN-23®, and Arachis oil.

Another embodiment comprises cyclosporin A, Rhodasurf ON-870®, and Arachis oil.

Another embodiment comprises cyclosporin A, Rhodasurf ON-877®, and Arachis oil.

Another embodiment comprises cyclosporin A, Rhodasurf TDA-8/5®, and Arachis oil.

Another embodiment comprises cyclosporin A, Ritapro 100®, and Arachis oil.

Another embodiment comprises cyclosporin A, Ritapro 165®, and Arachis oil.

Another embodiment comprises cyclosporin A, Ritapro 200®, and Arachis oil.

Another embodiment comprises cyclosporin A, Ritapro 300®, and Arachis oil.

Another embodiment comprises cyclosporin A, Ritoleth 10®, and Arachis oil.

Another embodiment comprises cyclosporin A, Ritoleth 2®, and Arachis oil.

Another embodiment comprises cyclosporin A, Ritoleth 20®, and Arachis oil.

Another embodiment comprises cyclosporin A, Ritoleth 5®, and Arachis oil.

Another embodiment comprises cyclosporin A, Santone 10-10-0®, and Arachis oil.

Another embodiment comprises cyclosporin A, Santone 3-1-S XTR®, and Arachis oil.

Another embodiment comprises cyclosporin A, Santone 3-1-SH®, and Arachis oil.

Another embodiment comprises cyclosporin A, Santone 8-1-O®, and Arachis oil.

Another embodiment comprises cyclosporin A, Solulan 16®, and Arachis oil.

Another embodiment comprises cyclosporin A, Solulan 75®, and Arachis oil.

Another embodiment comprises cyclosporin A, Solulan C-24®, and Arachis oil.

Another embodiment comprises cyclosporin A, Solulan L-575®, and Arachis oil.

Another embodiment comprises cyclosporin A, Standamox CAW®, and Arachis oil.

Another embodiment comprises cyclosporin A, Surfine AZI-A®, and Arachis oil.

Another embodiment comprises cyclosporin A, Surfonic CO-15®, and Arachis oil.

Another embodiment comprises cyclosporin A, Surfonic CO-25®, and Arachis oil.

Another embodiment comprises cyclosporin A, Surfonic CO-30®, and Arachis oil.

Another embodiment comprises cyclosporin A, Surfonic CO-36®, and Arachis oil.

Another embodiment comprises cyclosporin A, Surfonic CO-42®, and Arachis oil.

Another embodiment comprises cyclosporin A, Surfonic E400-MO®, and Arachis oil.

Another embodiment comprises cyclosporin A, Surfonic L12-3®, and Arachis oil.

Another embodiment comprises cyclosporin A, Surfonic L12-6®, and Arachis oil.

Another embodiment comprises cyclosporin A, Surfonic L12-8®, and Arachis oil.

Another embodiment comprises cyclosporin A, Surfonic L24-12®, and Arachis oil.

Another embodiment comprises cyclosporin A, Surfonic L24-2®, and Arachis oil.

Another embodiment comprises cyclosporin A, Surfonic L24-22®, and Arachis oil.

Another embodiment comprises cyclosporin A, Surfonic L24-3®, and Arachis oil.

Another embodiment comprises cyclosporin A, Surfonic L24-4®, and Arachis oil.

Another embodiment comprises cyclosporin A, Surfonic L4-5®, and Arachis oil.

Another embodiment comprises cyclosporin A, Surfonic L24-7®, and Arachis oil.

Another embodiment comprises cyclosporin A, Surfonic L24-7.1®, and Arachis oil.

Another embodiment comprises cyclosporin A, Surfonic L24-9®, and Arachis oil.

Another embodiment comprises cyclosporin A, Surfonic LF-17®, and Arachis oil.

Another embodiment comprises cyclosporin A, Surfonic LF-18®, and Arachis oil.

Another embodiment comprises cyclosporin A, Surfonic LF-37®, and Arachis oil.

Another embodiment comprises cyclosporin A, Surfonic LF-41®, and Arachis oil.

Another embodiment comprises cyclosporin A, Surfonic LF-42®, and Arachis oil.

Another embodiment comprises cyclosporin A, Surfonic LF-47®, and Arachis oil.

Another embodiment comprises cyclosporin A, Surfonic LF-68®, and Arachis oil.

Another embodiment comprises cyclosporin A, Surfonic N-10®, and Arachis oil.

Another embodiment comprises cyclosporin A, Surfonic N-100®, and Arachis oil.

Another embodiment comprises cyclosporin A, Surfonic N-102®, and Arachis oil.

Another embodiment comprises cyclosporin A, Surfonic N-120®, and Arachis oil.

Another embodiment comprises cyclosporin A, Surfonic N-150®, and Arachis oil.

Another embodiment comprises cyclosporin A, Surfonic N-200®, and Arachis oil.

Another embodiment comprises cyclosporin A, Surfonic N-300®, and Arachis oil.

Another embodiment comprises cyclosporin A, Surfonic N-31.5®, and Arachis oil.

Another embodiment comprises cyclosporin A, Surfonic N-40®, and Arachis oil.

Another embodiment comprises cyclosporin A, Surfonic N-400®, and Arachis oil.

Another embodiment comprises cyclosporin A, Surfonic N-60®, and Arachis oil.

Another embodiment comprises cyclosporin A, Surfonic N-85®, and Arachis oil.

Another embodiment comprises cyclosporin A, Surfonic N-95®, and Arachis oil.

Another embodiment comprises cyclosporin A, Surfonic NB-18®, and Arachis oil.

Another embodiment comprises cyclosporin A, Surfonic NB-23®, and Arachis oil.

Another embodiment comprises cyclosporin A, Surfonic NB-25®, and Arachis oil.

Another embodiment comprises cyclosporin A, Surfonic NB-307®, and Arachis oil.

Another embodiment comprises cyclosporin A, Surfonic NB-31®, and Arachis oil.

Another embodiment comprises cyclosporin A, Surfonic NB-41®, and Arachis oil.

Another embodiment comprises cyclosporin A, Surfonic NB-43®, and Arachis oil.

Another embodiment comprises cyclosporin A, Surfonic OP-100®, and Arachis oil.

Another embodiment comprises cyclosporin A, Surfonic OP-120®, and Arachis oil.

Another embodiment comprises cyclosporin A, Surfonic OP-15®, and Arachis oil.

Another embodiment comprises cyclosporin A, Surfonic OP-35®, and Arachis oil.

Another embodiment comprises cyclosporin A, Surfonic OP-70®, and Arachis oil.

Another embodiment comprises cyclosporin A, Surfonic P1®, and Arachis oil.

Another embodiment comprises cyclosporin A, Surfonic P3®, and Arachis oil.

Another embodiment comprises cyclosporin A, Surfonic P5®, and Arachis oil.

Another embodiment comprises cyclosporin A, Surfonic P6®, and Arachis oil.

Another embodiment comprises cyclosporin A, Surfonic POA-17R2®, and Arachis oil.

Another embodiment comprises cyclosporin A, Surfonic POA-25R2®, and Arachis oil.

Another embodiment comprises cyclosporin A, Surfonic TDA-11®, and Arachis oil.

Another embodiment comprises cyclosporin A, Surfonic TDA-3B®, and Arachis oil.

Another embodiment comprises cyclosporin A, Surfonic TDA-6®, and Arachis oil.

Another embodiment comprises cyclosporin A, Surfonic TDA-6/88®, and Arachis oil.

Another embodiment comprises cyclosporin A, Surfonic TDA-8®, and Arachis oil.

Another embodiment comprises cyclosporin A, Surfonic TDA-8/90®, and Arachis oil.

Another embodiment comprises cyclosporin A, Surfonic TDA-9®, and Arachis oil.

Another embodiment comprises cyclosporin A, Surfonic TX-CCN®, and Arachis oil.

Another embodiment comprises cyclosporin A, Tagat CH 40®, and Arachis oil.

Another embodiment comprises cyclosporin A, Tagat CH 60®, and Arachis oil.

Another embodiment comprises cyclosporin A, Tagat L 2®, and Arachis oil.

Another embodiment comprises cyclosporin A, Tagat S®, and Arachis oil.

Another embodiment comprises cyclosporin A, Tagat S 2®, and Arachis oil.

Another embodiment comprises cyclosporin A, Tally 100 Plus®, and Arachis oil.

Another embodiment comprises cyclosporin A, T-Det A106®, and Arachis oil.

Another embodiment comprises cyclosporin A, T-Det A109®, and Arachis oil.

Another embodiment comprises cyclosporin A, T-Det A136®, and Arachis oil.

Another embodiment comprises cyclosporin A, T-Det A139®, and Arachis oil.

Another embodiment comprises cyclosporin A, T-Det A2412®, and Arachis oil.

Another embodiment comprises cyclosporin A, T-Det A243®, and Arachis oil.

Another embodiment comprises cyclosporin A, T-Det A247®, and Arachis oil.

Another embodiment comprises cyclosporin A, T-Det A249®, and Arachis oil.

Another embodiment comprises cyclosporin A, T-Det A467®, and Arachis oil.

Another embodiment comprises cyclosporin A, T-Det COE®, and Arachis oil.

Another embodiment comprises cyclosporin A, T-Det DD-10®, and Arachis oil.

Another embodiment comprises cyclosporin A, T-Det DD-5®, and Arachis oil.

Another embodiment comprises cyclosporin A, T-Det DD-7®, and Arachis oil.

Another embodiment comprises cyclosporin A, T-Det EPO-104®, and Arachis oil.

Another embodiment comprises cyclosporin A, T-Det EPO-61®, and Arachis oil.

Another embodiment comprises cyclosporin A, T-Det EPO-62®, and Arachis oil.

Another embodiment comprises cyclosporin A, T-Det EPO-64®, and Arachis oil.

Another embodiment comprises cyclosporin A, T-Det N-1.5®, and Arachis oil.

Another embodiment comprises cyclosporin A, T-Det N-10.5®, and Arachis oil.

Another embodiment comprises cyclosporin A, T-Det N-100®, and Arachis oil.

Another embodiment comprises cyclosporin A, T-Det N-1007®, and Arachis oil.

Another embodiment comprises cyclosporin A, T-Det N-12®, and Arachis oil.

Another embodiment comprises cyclosporin A, T-Det N-14®, and Arachis oil.

Another embodiment comprises cyclosporin A, T-Det N-20®, and Arachis oil.

Another embodiment comprises cyclosporin A, T-Det N-30®, and Arachis oil.

Another embodiment comprises cyclosporin A, T-Det N-307®, and Arachis oil.

Another embodiment comprises cyclosporin A, T-Det N-4®, and Arachis oil.

Another embodiment comprises cyclosporin A, T-Det N-40®, and Arachis oil.

Another embodiment comprises cyclosporin A, T-Det N-407®, and Arachis oil.

Another embodiment comprises cyclosporin A, T-Det N-50®, and Arachis oil.

Another embodiment comprises cyclosporin A, T-Det N-507®, and Arachis oil.

Another embodiment comprises cyclosporin A, T-Det N-6®, and Arachis oil.

Another embodiment comprises cyclosporin A, T-Det N-70®, and Arachis oil.

Another embodiment comprises cyclosporin A, T-Det N-8®, and Arachis oil.

Another embodiment comprises cyclosporin A, T-Det N-9.5®, and Arachis oil.

Another embodiment comprises cyclosporin A, T-Det O-12®, and Arachis oil.

Another embodiment comprises cyclosporin A, T-Det O-165®, and Arachis oil.

Another embodiment comprises cyclosporin A, T-Det O-307®, and Arachis oil.

Another embodiment comprises cyclosporin A, T-Det O-40®, and Arachis oil.

Another embodiment comprises cyclosporin A, T-Det O-407®, and Arachis oil.

Another embodiment comprises cyclosporin A, T-Det O-6®, and Arachis oil.

Another embodiment comprises cyclosporin A, T-Det O-8®, and Arachis oil.

Another embodiment comprises cyclosporin A, T-Det O-9®, and Arachis oil.

Another embodiment comprises cyclosporin A, Tegin®, and Arachis oil.

Another embodiment comprises cyclosporin A, Tergitol 15-S-12®, and Arachis oil.

Another embodiment comprises cyclosporin A, Tergitol 15-S-15®, and Arachis oil.

Another embodiment comprises cyclosporin A, Tergitol 15-S-20®, and Arachis oil.

Another embodiment comprises cyclosporin A, Tergitol 15-S-20 (80AQ)®, and Arachis oil.

Another embodiment comprises cyclosporin A, Tergitol 15-S-3®, and Arachis oil.

Another embodiment comprises cyclosporin A, Tergitol 15-S-30®, and Arachis oil.

Another embodiment comprises cyclosporin A, Tergitol 15-S-40®, and Arachis oil.

Another embodiment comprises cyclosporin A, Tergitol 15-S-40 (70AQ)®, and Arachis oil.

Another embodiment comprises cyclosporin A, Tergitol 15-S-5®, and Arachis oil.

Another embodiment comprises cyclosporin A, Tergitol 15-S-7®, and Arachis oil.

Another embodiment comprises cyclosporin A, Tergitol 15-S-9®, and Arachis oil.

Another embodiment comprises cyclosporin A, Tergitol L-101®, and Arachis oil.

Another embodiment comprises cyclosporin A, Tergitol L-61®, and Arachis oil.

Another embodiment comprises cyclosporin A, Tergitol L-62®, and Arachis oil.

Another embodiment comprises cyclosporin A, Tergitol L-64®, and Arachis oil.

Another embodiment comprises cyclosporin A, Tergitol L-81®, and Arachis oil.

Another embodiment comprises cyclosporin A, Tergitol NP-10®, and Arachis oil.

Another embodiment comprises cyclosporin A, Tergitol NP-11®, and Arachis oil.

Another embodiment comprises cyclosporin A, Tergitol NP-12®, and Arachis oil.

Another embodiment comprises cyclosporin A, Tergitol NP-13®, and Arachis oil.

Another embodiment comprises cyclosporin A, Tergitol NP-15®, and Arachis oil.

Another embodiment comprises cyclosporin A, Tergitol NP-30®, and Arachis oil.

Another embodiment comprises cyclosporin A, Tergitol NP-30 (70 AQ)®, and Arachis oil.

Another embodiment comprises cyclosporin A, Tergitol NP-4®, and Arachis oil.

Another embodiment comprises cyclosporin A, Tergitol NP-40®, and Arachis oil.

Another embodiment comprises cyclosporin A, Tergitol NP-40 (70 AQ)®, and Arachis oil.

Another embodiment comprises cyclosporin A, Tergitol NP-50 (70 AQ)®, and Arachis oil.

Another embodiment comprises cyclosporin A, Tergitol NP-55 (70 AQ)®, and Arachis oil.

Another embodiment comprises cyclosporin A, Tergitol NP-6®, and Arachis oil.

Another embodiment comprises cyclosporin A, Tergitol NP-7®, and Arachis oil.

Another embodiment comprises cyclosporin A, Tergitol NP-70 (70 AQ)®, and Arachis oil.

Another embodiment comprises cyclosporin A, Tergitol NP-8®, and Arachis oil.

Another embodiment comprises cyclosporin A, Tergitol NP-9®, and Arachis oil.

Another embodiment comprises cyclosporin A, Tergitol NP-9.5®, and Arachis oil.

Another embodiment comprises cyclosporin A, Tergitol TMN-10®, and Arachis oil.

Another embodiment comprises cyclosporin A, Tergitol TMN-3®, and Arachis oil.

Another embodiment comprises cyclosporin A, Tergitol TMN-6®, and Arachis oil.

Another embodiment comprises cyclosporin A, Tergitol XD®, and Arachis oil.

Another embodiment comprises cyclosporin A, Tergitol XH®, and Arachis oil.

Another embodiment comprises cyclosporin A, Tergitol XJ®, and Arachis oil.

Another embodiment comprises cyclosporin A, Tetronic 1107®, and Arachis oil.

Another embodiment comprises cyclosporin A, Tetronic 1307®, and Arachis oil.

Another embodiment comprises cyclosporin A, Tetronic 150R1®, and Arachis oil.

Another embodiment comprises cyclosporin A, Tetronic 304®, and Arachis oil.

Another embodiment comprises cyclosporin A, Tetronic 701®, and Arachis oil.

Another embodiment comprises cyclosporin A, Tetronic 901®, and Arachis oil.

Another embodiment comprises cyclosporin A, Tetronic 904®, and Arachis oil.

Another embodiment comprises cyclosporin A, Tetronic 908®, and Arachis oil.

Another embodiment comprises cyclosporin A, Tetronic 90R4®, and Arachis oil.

Another embodiment comprises cyclosporin A, Tomadol 1-3®, and Arachis oil.

Another embodiment comprises cyclosporin A, Tomadol 1-5®, and Arachis oil.

Another embodiment comprises cyclosporin A, Tomadol 1-7®, and Arachis oil.

Another embodiment comprises cyclosporin A, Tomadol 23-1®, and Arachis oil.

Another embodiment comprises cyclosporin A, Tomadol 23-3®, and Arachis oil.

Another embodiment comprises cyclosporin A, Tomadol 23-5®, and Arachis oil.

Another embodiment comprises cyclosporin A, Tomadol 23-6.5®, and Arachis oil.

Another embodiment comprises cyclosporin A, Tomadol 25-12®, and Arachis oil.

Another embodiment comprises cyclosporin A, Tomadol 25-3®, and Arachis oil.

Another embodiment comprises cyclosporin A, Tomadol 25-7®, and Arachis oil.

Another embodiment comprises cyclosporin A, Tomadol 25-9®, and Arachis oil.

Another embodiment comprises cyclosporin A, Tomadol 45-13®, and Arachis oil.

Another embodiment comprises cyclosporin A, Tomadol 45-2.5®, and Arachis oil.

Another embodiment comprises cyclosporin A, Tomadol 45-7®, and Arachis oil.

Another embodiment comprises cyclosporin A, Tomadol 91-2.5®, and Arachis oil.

Another embodiment comprises cyclosporin A, Tomadol 91-6®, and Arachis oil.

Another embodiment comprises cyclosporin A, Tomadol 91-8®, and Arachis oil.

Another embodiment comprises cyclosporin A, Triton X-100®, and Arachis oil.

Another embodiment comprises cyclosporin A, Triton X-102®, and Arachis oil.

Another embodiment comprises cyclosporin A, Triton X-114®, and Arachis oil.

Another embodiment comprises cyclosporin A, Triton X-15®, and Arachis oil.

Another embodiment comprises cyclosporin A, Triton X-165 (70%)®, and Arachis oil.

Another embodiment comprises cyclosporin A, Triton X-207®, and Arachis oil.

Another embodiment comprises cyclosporin A, Triton X-305 (70%)®, and Arachis oil.

Another embodiment comprises cyclosporin A, Triton X-35®, and Arachis oil.

Another embodiment comprises cyclosporin A, Triton X-405 (70%)®, and Arachis oil.

Another embodiment comprises cyclosporin A, Triton X-45®, and Arachis oil.

Another embodiment comprises cyclosporin A, Triton X-705 (70%)®, and Arachis oil.

Another embodiment comprises cyclosporin A, Volpo S-10®, and Arachis oil.

Another embodiment comprises cyclosporin A, Volpo S-2®, and Arachis oil.

Another embodiment comprises cyclosporin A, Volpo S-20®, and Arachis oil.

Another embodiment comprises cyclosporin A, Volpo-10®, and Arachis oil.

Another embodiment comprises cyclosporin A, Volpo-20®, and Arachis oil.

Another embodiment comprises cyclosporin A, Volpo-3®, and Arachis oil.

Another embodiment comprises cyclosporin A, Volpo-5®, and Arachis oil.

Another embodiment comprises cyclosporin A, Abex 2515®, and Cottonseed oil.

Another embodiment comprises cyclosporin A, Abex 2525/40®, and Cottonseed oil.

Another embodiment comprises cyclosporin A, Abex 2535®, and Cottonseed oil.

Another embodiment comprises cyclosporin A, Abex 2545®, and Cottonseed oil.

Another embodiment comprises cyclosporin A, Acconon E®, and Cottonseed oil.

Another embodiment comprises cyclosporin A, Alfonic 1216CO-9®, and Cottonseed oil.

Another embodiment comprises cyclosporin A, Alfonic 1412-3®, and Cottonseed oil.

Another embodiment comprises cyclosporin A, Alfonic TDA-12®, and Cottonseed oil.

Another embodiment comprises cyclosporin A, Alfonic TDA-3®, and Cottonseed oil.

Another embodiment comprises cyclosporin A, Alfonic TDA-4®, and Cottonseed oil.

Another embodiment comprises cyclosporin A, Alfonic TDA-6®, and Cottonseed oil.

Another embodiment comprises cyclosporin A, Alfonic TDA-7®, and Cottonseed oil.

Another embodiment comprises cyclosporin A, Alfonic TDA-8®, and Cottonseed oil.

Another embodiment comprises cyclosporin A, Alfonic TDA-9®, and Cottonseed oil.

Another embodiment comprises cyclosporin A, Alkamuls 400-DO®, and Cottonseed oil.

Another embodiment comprises cyclosporin A, Alkamuls 400-MO/E®, and Cottonseed oil.

Another embodiment comprises cyclosporin A, Ameroxol OE-10®, and Cottonseed oil.

Another embodiment comprises cyclosporin A, Ameroxol OE-2®, and Cottonseed oil.

Another embodiment comprises cyclosporin A, Ameroxol OE-20®, and Cottonseed oil.

Another embodiment comprises cyclosporin A, Armix 176®, and Cottonseed oil.

Another embodiment comprises cyclosporin A, Armix 180-C®, and Cottonseed oil.

Another embodiment comprises cyclosporin A, Armix 183®, and Cottonseed oil.

Another embodiment comprises cyclosporin A, Brij 35®, and Cottonseed oil.

Another embodiment comprises cyclosporin A, Brij 35 Liquid®, and Cottonseed oil.

Another embodiment comprises cyclosporin A, Brij 35 SP®, and Cottonseed oil.

Another embodiment comprises cyclosporin A, Canasol MJ 2109®, and Cottonseed oil.

Another embodiment comprises cyclosporin A, Canasol MJ 52®, and Cottonseed oil.

Another embodiment comprises cyclosporin A, Canasol R 3600®, and Cottonseed oil.

Another embodiment comprises cyclosporin A, Canasol R 3603®, and Cottonseed oil.

Another embodiment comprises cyclosporin A, Canasol R 4000 H®, and Cottonseed oil.

Another embodiment comprises cyclosporin A, Cedepal CA-210®, and Cottonseed oil.

Another embodiment comprises cyclosporin A, Cedepal CA-520®, and Cottonseed oil.

Another embodiment comprises cyclosporin A, Cedepal CA-630®, and Cottonseed oil.

Another embodiment comprises cyclosporin A, Cedepal CA-720®, and Cottonseed oil.

Another embodiment comprises cyclosporin A, Cedepal CA-890®, and Cottonseed oil.

Another embodiment comprises cyclosporin A, Cedepal CA-897®, and Cottonseed oil.

Another embodiment comprises cyclosporin A, Cedepal CO-210®, and Cottonseed oil.

Another embodiment comprises cyclosporin A, Cedepal CO-430®, and Cottonseed oil.

Another embodiment comprises cyclosporin A, Cedepal CO-530®, and Cottonseed oil.

Another embodiment comprises cyclosporin A, Cedepal CO-630®, and Cottonseed oil.

Another embodiment comprises cyclosporin A, Cedepal CO-710®, and Cottonseed oil.

Another embodiment comprises cyclosporin A, Cedepal CO-730®, and Cottonseed oil.

Another embodiment comprises cyclosporin A, Cedepal CO-880®, and Cottonseed oil.

Another embodiment comprises cyclosporin A, Cedepal CO-887®, and Cottonseed oil.

Another embodiment comprises cyclosporin A, Cedepal CO-890®, and Cottonseed oil.

Another embodiment comprises cyclosporin A, Cedepal CO-897®, and Cottonseed oil.

Another embodiment comprises cyclosporin A, Cedepal CO-970®, and Cottonseed oil.

Another embodiment comprises cyclosporin A, Cedepal CO-977®, and Cottonseed oil.

Another embodiment comprises cyclosporin A, Cedepal CO-990®, and Cottonseed oil.

Another embodiment comprises cyclosporin A, Cedepal CO-997®, and Cottonseed oil.

Another embodiment comprises cyclosporin A, Chemal BP 261®, and Cottonseed oil.

Another embodiment comprises cyclosporin A, Chemal BP 262®, and Cottonseed oil.

Another embodiment comprises cyclosporin A, Chemal BP 3172®, and Cottonseed oil.

Another embodiment comprises cyclosporin A, Chemal BP 3174®, and Cottonseed oil.

Another embodiment comprises cyclosporin A, Chemal DA-4®, and Cottonseed oil.

Another embodiment comprises cyclosporin A, Chemal DA-6®, and Cottonseed oil.

Another embodiment comprises cyclosporin A, Chemal DA-9®, and Cottonseed oil.

Another embodiment comprises cyclosporin A, Chemal LA-23®, and Cottonseed oil.

Another embodiment comprises cyclosporin A, Chemal LA-4®, and Cottonseed oil.

Another embodiment comprises cyclosporin A, Chemal LA-9®, and Cottonseed oil.

Another embodiment comprises cyclosporin A, Chemal OA-20G®, and Cottonseed oil.

Another embodiment comprises cyclosporin A, Chemal OA-5®, and Cottonseed oil.

Another embodiment comprises cyclosporin A, Chemal OA-9®, and Cottonseed oil.

Another embodiment comprises cyclosporin A, Chemal TDA-12®, and Cottonseed oil.

Another embodiment comprises cyclosporin A, Chemal TDA-15®, and Cottonseed oil.

Another embodiment comprises cyclosporin A, Chemal TDA-3®, and Cottonseed oil.

Another embodiment comprises cyclosporin A, Chemal TDA-6®, and Cottonseed oil.

Another embodiment comprises cyclosporin A, Chemal TDA-9®, and Cottonseed oil.

Another embodiment comprises cyclosporin A, Chemax CO-16®, and Cottonseed oil.

Another embodiment comprises cyclosporin A, Chemax CO-200/50®, and Cottonseed oil.

Another embodiment comprises cyclosporin A, Chemax CO-25®, and Cottonseed oil.

Another embodiment comprises cyclosporin A, Chemax CO-30®, and Cottonseed oil.

Another embodiment comprises cyclosporin A, Chemax CO-36®, and Cottonseed oil.

Another embodiment comprises cyclosporin A, Chemax CO-40®, and Cottonseed oil.

Another embodiment comprises cyclosporin A, Chemax CO-5®, and Cottonseed oil.

Another embodiment comprises cyclosporin A, Chemax CO-80®, and Cottonseed oil.

Another embodiment comprises cyclosporin A, Chemax DNP-150/50®, and Cottonseed oil.

Another embodiment comprises cyclosporin A, Chemax DNP-18®, and Cottonseed oil.

Another embodiment comprises cyclosporin A, Chemax DNP-8®, and Cottonseed oil.

Another embodiment comprises cyclosporin A, Chemax E 200 ML®, and Cottonseed oil.

Another embodiment comprises cyclosporin A, Chemax E 200 MO®, and Cottonseed oil.

Another embodiment comprises cyclosporin A, Chemax E 400 ML®, and Cottonseed oil.

Another embodiment comprises cyclosporin A, Chemax E 400 MO®, and Cottonseed oil.

Another embodiment comprises cyclosporin A, Chemax E 600 ML®, and Cottonseed oil.

Another embodiment comprises cyclosporin A, Chemax E 600 MO®, and Cottonseed oil.

Another embodiment comprises cyclosporin A, Chemax E1000 MO®, and Cottonseed oil.

Another embodiment comprises cyclosporin A, Chemax E1000 MS®, and Cottonseed oil.

Another embodiment comprises cyclosporin A, Chemax E200 MS®, and Cottonseed oil.

Another embodiment comprises cyclosporin A, Chemax E400 MS®, and Cottonseed oil.

Another embodiment comprises cyclosporin A, Chemax E600 MS®, and Cottonseed oil.

Another embodiment comprises cyclosporin A, Chemax HCO-16®, and Cottonseed oil.

Another embodiment comprises cyclosporin A, Chemax HCO-200/50®, and Cottonseed oil.

Another embodiment comprises cyclosporin A, Chemax HCO-25®, and Cottonseed oil.

Another embodiment comprises cyclosporin A, Chemax HCO-5®, and Cottonseed oil.

Another embodiment comprises cyclosporin A, Chemax TO-16®, and Cottonseed oil.

Another embodiment comprises cyclosporin A, Chemax TO-8®, and Cottonseed oil.

Another embodiment comprises cyclosporin A, Colalube 3409®, and Cottonseed oil.

Another embodiment comprises cyclosporin A, Colalube 3411®, and Cottonseed oil.

Another embodiment comprises cyclosporin A, Colalube 3413®, and Cottonseed oil.

Another embodiment comprises cyclosporin A, Colalube 3414®, and Cottonseed oil.

Another embodiment comprises cyclosporin A, Colalube 3415®, and Cottonseed oil.

Another embodiment comprises cyclosporin A, Colalube 3416®, and Cottonseed oil.

Another embodiment comprises cyclosporin A, Colalube 3417®, and Cottonseed oil.

Another embodiment comprises cyclosporin A, Delonic NPE-100®, and Cottonseed oil.

Another embodiment comprises cyclosporin A, Delonic NPE-30®, and Cottonseed oil.

Another embodiment comprises cyclosporin A, Delonic NPE-40®, and Cottonseed oil.

Another embodiment comprises cyclosporin A, Delonic OPE-10®, and Cottonseed oil.

Another embodiment comprises cyclosporin A, Delonic OPE-12®, and Cottonseed oil.

Another embodiment comprises cyclosporin A, Delonic OPE-30®, and Cottonseed oil.

Another embodiment comprises cyclosporin A, Delonic OPE-40®, and Cottonseed oil.

Another embodiment comprises cyclosporin A, Delonic OPE-5®, and Cottonseed oil.

Another embodiment comprises cyclosporin A, Delonic OPE-7.5®, and Cottonseed oil.

Another embodiment comprises cyclosporin A, DePeg 16-CO®, and Cottonseed oil.

Another embodiment comprises cyclosporin A, DePeg 25-CO®, and Cottonseed oil.

Another embodiment comprises cyclosporin A, DePeg 30-CO®, and Cottonseed oil.

Another embodiment comprises cyclosporin A, DePeg 40-CO®, and Cottonseed oil.

Another embodiment comprises cyclosporin A, DePeg 5-CO®, and Cottonseed oil.

Another embodiment comprises cyclosporin A, DePeg 80-CO®, and Cottonseed oil.

Another embodiment comprises cyclosporin A, DeSonic S-100®, and Cottonseed oil.

Another embodiment comprises cyclosporin A, DeSonic S-114®, and Cottonseed oil.

Another embodiment comprises cyclosporin A, DeSonic S-405®, and Cottonseed oil.

Another embodiment comprises cyclosporin A, DeSonic S-45®, and Cottonseed oil.

Another embodiment comprises cyclosporin A, DeSonic TDA-9®, and Cottonseed oil.

Another embodiment comprises cyclosporin A, DeThox Acid O-14®, and Cottonseed oil.

Another embodiment comprises cyclosporin A, DeThox Acid O-16®, and Cottonseed oil.

Another embodiment comprises cyclosporin A, DeThox Acid O-9®, and Cottonseed oil.

Another embodiment comprises cyclosporin A, DeThox Acid TO-14®, and Cottonseed oil.

Another embodiment comprises cyclosporin A, DeThox Acid TO-16.5®, and Cottonseed oil.

Another embodiment comprises cyclosporin A, DeThox Acid TO-8.5®, and Cottonseed oil.

Another embodiment comprises cyclosporin A, Dur-Em 114®, and Cottonseed oil.

Another embodiment comprises cyclosporin A, Dur-Em 117®, and Cottonseed oil.

Another embodiment comprises cyclosporin A, Dur-Em 204®, and Cottonseed oil.

Another embodiment comprises cyclosporin A, Dur-Em 207®, and Cottonseed oil.

Another embodiment comprises cyclosporin A, Dur-Em 207E®, and Cottonseed oil.

Another embodiment comprises cyclosporin A, Emerest 2704®, and Cottonseed oil.

Another embodiment comprises cyclosporin A, Emerest 2712®, and Cottonseed oil.

Another embodiment comprises cyclosporin A, Emerest 2715®, and Cottonseed oil.

Another embodiment comprises cyclosporin A, Ethal EH-2®, and Cottonseed oil.

Another embodiment comprises cyclosporin A, Ethal EH-5®, and Cottonseed oil.

Another embodiment comprises cyclosporin A, Ethal OA-23®, and Cottonseed oil.

Another embodiment comprises cyclosporin A, Ethosperse CA-20®, and Cottonseed oil.

Another embodiment comprises cyclosporin A, Ethosperse LA-12®, and Cottonseed oil.

Another embodiment comprises cyclosporin A, Ethosperse LA-23®, and Cottonseed oil.

Another embodiment comprises cyclosporin A, Ethosperse LA-4®, and Cottonseed oil.

Another embodiment comprises cyclosporin A, Ethox 3095®, and Cottonseed oil.

Another embodiment comprises cyclosporin A, Ethox CO-16®, and Cottonseed oil.

Another embodiment comprises cyclosporin A, Ethox CO-200®, and Cottonseed oil.

Another embodiment comprises cyclosporin A, Ethox CO-25®, and Cottonseed oil.

Another embodiment comprises cyclosporin A, Ethox CO-30®, and Cottonseed oil.

Another embodiment comprises cyclosporin A, Ethox CO-36®, and Cottonseed oil.

Another embodiment comprises cyclosporin A, Ethox CO-40®, and Cottonseed oil.

Another embodiment comprises cyclosporin A, Ethox CO-5®, and Cottonseed oil.

Another embodiment comprises cyclosporin A, Ethox DO-14®, and Cottonseed oil.

Another embodiment comprises cyclosporin A, Ethox DO-9®, and Cottonseed oil.

Another embodiment comprises cyclosporin A, Ethox MA-8®, and Cottonseed oil.

Another embodiment comprises cyclosporin A, Ethox MI-14®, and Cottonseed oil.

Another embodiment comprises cyclosporin A, Ethox ML-14®, and Cottonseed oil.

Another embodiment comprises cyclosporin A, Ethox ML-5®, and Cottonseed oil.

Another embodiment comprises cyclosporin A, Ethox MO-14®, and Cottonseed oil.

Another embodiment comprises cyclosporin A, Ethox MO-9®, and Cottonseed oil.

Another embodiment comprises cyclosporin A, Ethox MS-14®, and Cottonseed oil.

Another embodiment comprises cyclosporin A, Ethox MS-23®, and Cottonseed oil.

Another embodiment comprises cyclosporin A, Ethox MS-40®, and Cottonseed oil.

Another embodiment comprises cyclosporin A, Ethox MS-8®, and Cottonseed oil.

Another embodiment comprises cyclosporin A, Ethox TO-16®, and Cottonseed oil.

Another embodiment comprises cyclosporin A, Ethox TO-8®, and Cottonseed oil.

Another embodiment comprises cyclosporin A, Ethylan SN-120®, and Cottonseed oil.

Another embodiment comprises cyclosporin A, Ethylan SN-70®, and Cottonseed oil.

Another embodiment comprises cyclosporin A, Ethylan SN-90®, and Cottonseed oil.

Another embodiment comprises cyclosporin A, Flexricin 13®, and Cottonseed oil.

Another embodiment comprises cyclosporin A, Flexricin 15®, and Cottonseed oil.

Another embodiment comprises cyclosporin A, Genapol 1261®, and Cottonseed oil.

Another embodiment comprises cyclosporin A, Generol 122 E25®, and Cottonseed oil.

Another embodiment comprises cyclosporin A, Glicepol 160®, and Cottonseed oil.

Another embodiment comprises cyclosporin A, Glicepol 560®, and Cottonseed oil.

Another embodiment comprises cyclosporin A, Glicepol GMS 20®, and Cottonseed oil.

Another embodiment comprises cyclosporin A, Hetoxide BN-13®, and Cottonseed oil.

Another embodiment comprises cyclosporin A, Hetoxide DNP-4®, and Cottonseed oil.

Another embodiment comprises cyclosporin A, Hetoxide DNP-9.6®, and Cottonseed oil.

Another embodiment comprises cyclosporin A, Hetoxide G-26®, and Cottonseed oil.

Another embodiment comprises cyclosporin A, Hetoxide G-7®, and Cottonseed oil.

Another embodiment comprises cyclosporin A, Hetoxide GT-20®, and Cottonseed oil.

Another embodiment comprises cyclosporin A, Hetoxide HC-40®, and Cottonseed oil.

Another embodiment comprises cyclosporin A, Hetoxol C-24®, and Cottonseed oil.

Another embodiment comprises cyclosporin A, Hetoxol CA-10®, and Cottonseed oil.

Another embodiment comprises cyclosporin A, Hetoxol CA-2®, and Cottonseed oil.

Another embodiment comprises cyclosporin A, Hetoxol CA-20®, and Cottonseed oil.

Another embodiment comprises cyclosporin A, Hetoxol CAWS®, and Cottonseed oil.

Another embodiment comprises cyclosporin A, Hetoxol CD-3®, and Cottonseed oil.

Another embodiment comprises cyclosporin A, Hetoxol CD-4®, and Cottonseed oil.

Another embodiment comprises cyclosporin A, Hetoxol CS-15®, and Cottonseed oil.

Another embodiment comprises cyclosporin A, Hetoxol CS-20®, and Cottonseed oil.

Another embodiment comprises cyclosporin A, Hetoxol CS-20D®, and Cottonseed oil.

Another embodiment comprises cyclosporin A, Hetoxol CS-25®, and Cottonseed oil.

Another embodiment comprises cyclosporin A, Hetoxol CS-50®, and Cottonseed oil.

Another embodiment comprises cyclosporin A, Hetoxol CS-9®, and Cottonseed oil.

Another embodiment comprises cyclosporin A, Hetoxol CSA-15®, and Cottonseed oil.

Another embodiment comprises cyclosporin A, Hetoxol D®, and Cottonseed oil.

Another embodiment comprises cyclosporin A, Hetoxol G®, and Cottonseed oil.

Another embodiment comprises cyclosporin A, Hetoxol J®, and Cottonseed oil.

Another embodiment comprises cyclosporin A, Hetoxol L®, and Cottonseed oil.

Another embodiment comprises cyclosporin A, Hetoxol L-23®, and Cottonseed oil.

Another embodiment comprises cyclosporin A, Hetoxol L-4®, and Cottonseed oil.

Another embodiment comprises cyclosporin A, Hetoxol L-9®, and Cottonseed oil.

Another embodiment comprises cyclosporin A, Hetoxol LS-9®, and Cottonseed oil.

Another embodiment comprises cyclosporin A, Hetoxol M-3®, and Cottonseed oil.

Another embodiment comprises cyclosporin A, Hetoxol OA-10 Special®, and Cottonseed oil.

Another embodiment comprises cyclosporin A, Hetoxol OA-20 Special®, and Cottonseed oil.

Another embodiment comprises cyclosporin A, Hetoxol OA-3 Special®, and Cottonseed oil.

Another embodiment comprises cyclosporin A, Hetoxol OA-5 Special®, and Cottonseed oil.

Another embodiment comprises cyclosporin A, Hetoxol OL-10®, and Cottonseed oil.

Another embodiment comprises cyclosporin A, Hetoxol OL-2®, and Cottonseed oil.

Another embodiment comprises cyclosporin A, Hetoxol OL-23®, and Cottonseed oil.

Another embodiment comprises cyclosporin A, Hetoxol OL-4®, and Cottonseed oil.

Another embodiment comprises cyclosporin A, Hetoxol OL-40®, and Cottonseed oil.

Another embodiment comprises cyclosporin A, Hetoxol OL-5®, and Cottonseed oil.

Another embodiment comprises cyclosporin A, Hetoxol PLA®, and Cottonseed oil.

Another embodiment comprises cyclosporin A, Hetoxol SP-15®, and Cottonseed oil.

Another embodiment comprises cyclosporin A, Hetoxol STA-10®, and Cottonseed oil.

Another embodiment comprises cyclosporin A, Hetoxol STA-2®, and Cottonseed oil.

Another embodiment comprises cyclosporin A, Hetoxol STA-30®, and Cottonseed oil.

Another embodiment comprises cyclosporin A, Hetoxol TD-12®, and Cottonseed oil.

Another embodiment comprises cyclosporin A, Hetoxol TD-18®, and Cottonseed oil.

Another embodiment comprises cyclosporin A, Hetoxol TD-3®, and Cottonseed oil.

Another embodiment comprises cyclosporin A, Hetoxol TD-6®, and Cottonseed oil.

Another embodiment comprises cyclosporin A, Hetoxol TD-9®, and Cottonseed oil.

Another embodiment comprises cyclosporin A, Hetoxol TDEP-15®, and Cottonseed oil.

Another embodiment comprises cyclosporin A, Hetoxol TDEP-63®, and Cottonseed oil.

Another embodiment comprises cyclosporin A, Iconol 24-12®, and Cottonseed oil.

Another embodiment comprises cyclosporin A, Iconol 24-23®, and Cottonseed oil.

Another embodiment comprises cyclosporin A, Iconol 24-4®, and Cottonseed oil.

Another embodiment comprises cyclosporin A, Iconol DA-4®, and Cottonseed oil.

Another embodiment comprises cyclosporin A, Iconol DA-6®, and Cottonseed oil.

Another embodiment comprises cyclosporin A, Iconol TDA-10®, and Cottonseed oil.

Another embodiment comprises cyclosporin A, Iconol TDA-3®, and Cottonseed oil.

Another embodiment comprises cyclosporin A, Iconol TDA-6®, and Cottonseed oil.

Another embodiment comprises cyclosporin A, Iconol TDA-8-90%®, and Cottonseed oil.

Another embodiment comprises cyclosporin A, Iconol TDA-9®, and Cottonseed oil.

Another embodiment comprises cyclosporin A, Igepal CA-210®, and Cottonseed oil.

Another embodiment comprises cyclosporin A, Igepal CA-407®, and Cottonseed oil.

Another embodiment comprises cyclosporin A, Igepal CA-420®, and Cottonseed oil.

Another embodiment comprises cyclosporin A, Igepal CA-520®, and Cottonseed oil.

Another embodiment comprises cyclosporin A, Igepal CA-620®, and Cottonseed oil.

Another embodiment comprises cyclosporin A, Igepal CA-630®, and Cottonseed oil.

Another embodiment comprises cyclosporin A, Igepal CA-720®, and Cottonseed oil.

Another embodiment comprises cyclosporin A, Igepal CA-877®, and Cottonseed oil.

Another embodiment comprises cyclosporin A, Igepal CA-887®, and Cottonseed oil.

Another embodiment comprises cyclosporin A, Igepal CA-897®, and Cottonseed oil.

Another embodiment comprises cyclosporin A, Igepal CO-210®, and Cottonseed oil.

Another embodiment comprises cyclosporin A, Igepal CO-430®, and Cottonseed oil.

Another embodiment comprises cyclosporin A, Igepal CO-520®, and Cottonseed oil.

Another embodiment comprises cyclosporin A, Igepal CO-530®, and Cottonseed oil.

Another embodiment comprises cyclosporin A, Igepal CO-610®, and Cottonseed oil.

Another embodiment comprises cyclosporin A, Igepal CO-630®, and Cottonseed oil.

Another embodiment comprises cyclosporin A, Igepal CO-630 Special®, and Cottonseed oil.

Another embodiment comprises cyclosporin A, Igepal CO-660®, and Cottonseed oil.

Another embodiment comprises cyclosporin A, Igepal CO-710®, and Cottonseed oil.

Another embodiment comprises cyclosporin A, Igepal CO-720®, and Cottonseed oil.

Another embodiment comprises cyclosporin A, Igepal CO-730®, and Cottonseed oil.

Another embodiment comprises cyclosporin A, lgepal CO-738®, and Cottonseed oil.

Another embodiment comprises cyclosporin A, Igepal CO-850®, and Cottonseed oil.

Another embodiment comprises cyclosporin A, lgepal CO-880®, and Cottonseed oil.

Another embodiment comprises cyclosporin A, Igepal CO-887®, and Cottonseed oil.

Another embodiment comprises cyclosporin A, Igepal CO-890®, and Cottonseed oil.

Another embodiment comprises cyclosporin A, Igepal CO-897®, and Cottonseed oil.

Another embodiment comprises cyclosporin A, Igepal CO-970®, and Cottonseed oil.

Another embodiment comprises cyclosporin A, lgepal CO-977®, and Cottonseed oil.

Another embodiment comprises cyclosporin A, Igepal CO-987®, and Cottonseed oil.

Another embodiment comprises cyclosporin A, Igepal CO-990 FLK®, and Cottonseed oil.

Another embodiment comprises cyclosporin A, Igepal CO-997®, and Cottonseed oil.

Another embodiment comprises cyclosporin A, Igepal DM-430®, and Cottonseed oil.

Another embodiment comprises cyclosporin A, Igepal DM-530®, and Cottonseed oil.

Another embodiment comprises cyclosporin A, Igepal DM-710®, and Cottonseed oil.

Another embodiment comprises cyclosporin A, Igepal DM-730®, and Cottonseed oil.

Another embodiment comprises cyclosporin A, Igepal DM-880®, and Cottonseed oil.

Another embodiment comprises cyclosporin A, Igepal DM-970 FLK®, and Cottonseed oil.

Another embodiment comprises cyclosporin A, Igepal OD-410®, and Cottonseed oil.

Another embodiment comprises cyclosporin A, Igepal RC-520®, and Cottonseed oil.

Another embodiment comprises cyclosporin A, Igepal RC-620®, and Cottonseed oil.

Another embodiment comprises cyclosporin A, Igepal RC-630®, and Cottonseed oil.

Another embodiment comprises cyclosporin A, Lexemul CS-20®, and Cottonseed oil.

Another embodiment comprises cyclosporin A, Lipo GMS 450®, and Cottonseed oil.

Another embodiment comprises cyclosporin A, Lipo Polyglycol 1000®, and Cottonseed oil.

Another embodiment comprises cyclosporin A, Lipo Polyglycol 200®, and Cottonseed oil.

Another embodiment comprises cyclosporin A, Lipo Polyglycol 300®, and Cottonseed oil.

Another embodiment comprises cyclosporin A, Lipo Polyglycol 3350®, and Cottonseed oil.

Another embodiment comprises cyclosporin A, Lipo Polyglycol 400®, and Cottonseed oil.

Another embodiment comprises cyclosporin A, Lipo Polyglycol 600®, and Cottonseed oil.

Another embodiment comprises cyclosporin A, Lipocol C-10®, and Cottonseed oil.

Another embodiment comprises cyclosporin A, Lipocol C-2®, and Cottonseed oil.

Another embodiment comprises cyclosporin A, Lipocol C-20®, and Cottonseed oil.

Another embodiment comprises cyclosporin A, Lipocol L-12®, and Cottonseed oil.

Another embodiment comprises cyclosporin A, Lipocol L-23®, and Cottonseed oil.

Another embodiment comprises cyclosporin A, Lipocol L-4®, and Cottonseed oil.

Another embodiment comprises cyclosporin A, Lipocol O-10®, and Cottonseed oil.

Another embodiment comprises cyclosporin A, Lipocol O-2®, and Cottonseed oil.

Another embodiment comprises cyclosporin A, Lipocol O-20®, and Cottonseed oil.

Another embodiment comprises cyclosporin A, Lipocol O-5®, and Cottonseed oil.

Another embodiment comprises cyclosporin A, Lipocol S-10®, and Cottonseed oil.

Another embodiment comprises cyclosporin A, Lipocol S-2®, and Cottonseed oil.

Another embodiment comprises cyclosporin A, Lipocol S-20®, and Cottonseed oil.

Another embodiment comprises cyclosporin A, Lipocol SC-15®, and Cottonseed oil.

Another embodiment comprises cyclosporin A, Lipocol SC-20®, and Cottonseed oil.

Another embodiment comprises cyclosporin A, Lipocol SC-4®, and Cottonseed oil.

Another embodiment comprises cyclosporin A, Lipocol TD-12®, and Cottonseed oil.

Another embodiment comprises cyclosporin A, Lumulse 40-O K®, and Cottonseed oil.

Another embodiment comprises cyclosporin A, Lumulse 42-O K®, and Cottonseed oil.

Another embodiment comprises cyclosporin A, Lumulse GMT-40®, and Cottonseed oil.

Another embodiment comprises cyclosporin A, Lumulse PEG 1450®, and Cottonseed oil.

Another embodiment comprises cyclosporin A, Lumulse PEG 1450 NF®, and Cottonseed oil.

Another embodiment comprises cyclosporin A, Lumulse PEG 8000®, and Cottonseed oil.

Another embodiment comprises cyclosporin A, Lumulse PGO®, and Cottonseed oil.

Another embodiment comprises cyclosporin A, Lumulse POE (2) Oleyl Amine®, and Cottonseed oil.

Another embodiment comprises cyclosporin A, Magrabar PGO-104®, and Cottonseed oil.

Another embodiment comprises cyclosporin A, Mapeg 200 ML®, and Cottonseed oil.

Another embodiment comprises cyclosporin A, Mapeg 400 MO®, and Cottonseed oil.

Another embodiment comprises cyclosporin A, Mapeg 400 MOT®, and Cottonseed oil.

Another embodiment comprises cyclosporin A, Mapeg 400DO®, and Cottonseed oil.

Another embodiment comprises cyclosporin A, Mapeg 400DOT®, and Cottonseed oil.

Another embodiment comprises cyclosporin A, Mapeg 600DOT®, and Cottonseed oil.

Another embodiment comprises cyclosporin A, Marlox B 24/80®, and Cottonseed oil.

Another embodiment comprises cyclosporin A, Marlox FK 86®, and Cottonseed oil.

Another embodiment comprises cyclosporin A, Marlox MO 154®, and Cottonseed oil.

Another embodiment comprises cyclosporin A, Mazox CDA CG®, and Cottonseed oil.

Another embodiment comprises cyclosporin A, Monoglycerides Glyceryl Monestearate®, and Cottonseed oil.

Another embodiment comprises cyclosporin A, Myrj 45®, and Cottonseed oil.

Another embodiment comprises cyclosporin A, Myrj 52®, and Cottonseed oil.

Another embodiment comprises cyclosporin A, Myrj 52S®, and Cottonseed oil.

Another embodiment comprises cyclosporin A, Myrj 53®, and Cottonseed oil.

Another embodiment comprises cyclosporin A, Myrj 59®, and Cottonseed oil.

Another embodiment comprises cyclosporin A, Norfox NP-12®, and Cottonseed oil.

Another embodiment comprises cyclosporin A, Norfox NP-4®, and Cottonseed oil.

Another embodiment comprises cyclosporin A, Norfox NP-6®, and Cottonseed oil.

Another embodiment comprises cyclosporin A, Norfox NP-887®, and Cottonseed oil.

Another embodiment comprises cyclosporin A, Norfox NP-9®, and Cottonseed oil.

Another embodiment comprises cyclosporin A, Norfox NP-977®, and Cottonseed oil.

Another embodiment comprises cyclosporin A, Norfox OP-100®, and Cottonseed oil.

Another embodiment comprises cyclosporin A, Norfox OP-102®, and Cottonseed oil.

Another embodiment comprises cyclosporin A, Norfox OP-114®, and Cottonseed oil.

Another embodiment comprises cyclosporin A, Norfox OP-45®, and Cottonseed oil.

Another embodiment comprises cyclosporin A, Pegosperse 100-L®, and Cottonseed oil.

Another embodiment comprises cyclosporin A, Pegosperse 100-O®, and Cottonseed oil.

Another embodiment comprises cyclosporin A, Pegosperse 100-S®, and Cottonseed oil.

Another embodiment comprises cyclosporin A, Pegosperse 1500-MS®, and Cottonseed oil.

Another embodiment comprises cyclosporin A, Pegosperse 1750-MS®, and Cottonseed oil.

Another embodiment comprises cyclosporin A, Pegosperse 200 DL®, and Cottonseed oil.

Another embodiment comprises cyclosporin A, Pegosperse 200-ML®, and Cottonseed oil.

Another embodiment comprises cyclosporin A, Pegosperse 400-DL®, and Cottonseed oil.

Another embodiment comprises cyclosporin A, Pegosperse 400-DO®, and Cottonseed oil.

Another embodiment comprises cyclosporin A, Pegosperse 400-DS®, and Cottonseed oil.

Another embodiment comprises cyclosporin A, Pegosperse 400-ML®, and Cottonseed oil.

Another embodiment comprises cyclosporin A, Pegosperse 400-MO®, and Cottonseed oil.

Another embodiment comprises cyclosporin A, Pegosperse 400-MOT®, and Cottonseed oil.

Another embodiment comprises cyclosporin A, Pegosperse 400-MS®, and Cottonseed oil.

Another embodiment comprises cyclosporin A, Pegosperse 50-DS®, and Cottonseed oil.

Another embodiment comprises cyclosporin A, Pegosperse 50-MS®, and Cottonseed oil.

Another embodiment comprises cyclosporin A, Pegosperse 600-ML®, and Cottonseed oil.

Another embodiment comprises cyclosporin A, Pegosperse 600-MS®, and Cottonseed oil.

Another embodiment comprises cyclosporin A, Plurafac A-38®, and Cottonseed oil.

Another embodiment comprises cyclosporin A, Plurafac B-25-5®, and Cottonseed oil.

Another embodiment comprises cyclosporin A, Plurafac B-26®, and Cottonseed oil.

Another embodiment comprises cyclosporin A, Plurafac D-25®, and Cottonseed oil.

Another embodiment comprises cyclosporin A, Plurafac LF 131®, and Cottonseed oil.

Another embodiment comprises cyclosporin A, Plurafac LF 4030®, and Cottonseed oil.

Another embodiment comprises cyclosporin A, Plurafac LF 7000®, and Cottonseed oil.

Another embodiment comprises cyclosporin A, Plurafac RA-20®, and Cottonseed oil.

Another embodiment comprises cyclosporin A, Plurafac RA-30®, and Cottonseed oil.

Another embodiment comprises cyclosporin A, Plurafac RA-40®, and Cottonseed oil.

Another embodiment comprises cyclosporin A, Pluronic 10R5®, and Cottonseed oil.

Another embodiment comprises cyclosporin A, Pluronic 17R2®, and Cottonseed oil.

Another embodiment comprises cyclosporin A, Pluronic 17R4®, and Cottonseed oil.

Another embodiment comprises cyclosporin A, Pluronic 25R2®, and Cottonseed oil.

Another embodiment comprises cyclosporin A, Pluronic 25R4®, and Cottonseed oil.

Another embodiment comprises cyclosporin A, Pluronic 31R1®, and Cottonseed oil.

Another embodiment comprises cyclosporin A, Pluronic F108®, and Cottonseed oil.

Another embodiment comprises cyclosporin A, Pluronic F127®, and Cottonseed oil.

Another embodiment comprises cyclosporin A, Pluronic F38®, and Cottonseed oil.

Another embodiment comprises cyclosporin A, Pluronic F68®, and Cottonseed oil.

Another embodiment comprises cyclosporin A, Pluronic F68LF®, and Cottonseed oil.

Another embodiment comprises cyclosporin A, Pluronic F77®, and Cottonseed oil.

Another embodiment comprises cyclosporin A, Pluronic F87®, and Cottonseed oil.

Another embodiment comprises cyclosporin A, Pluronic F88®, and Cottonseed oil.

Another embodiment comprises cyclosporin A, Pluronic F98®, and Cottonseed oil.

Another embodiment comprises cyclosporin A, Pluronic L10®, and Cottonseed oil.

Another embodiment comprises cyclosporin A, Pluronic L101®, and Cottonseed oil.

Another embodiment comprises cyclosporin A, Pluronic L121®, and Cottonseed oil.

Another embodiment comprises cyclosporin A, Pluronic L31®, and Cottonseed oil.

Another embodiment comprises cyclosporin A, Pluronic L35®, and Cottonseed oil.

Another embodiment comprises cyclosporin A, Pluronic L43®, and Cottonseed oil.

Another embodiment comprises cyclosporin A, Pluronic L44®, and Cottonseed oil.

Another embodiment comprises cyclosporin A, Pluronic L61®, and Cottonseed oil.

Another embodiment comprises cyclosporin A, Pluronic L62®, and Cottonseed oil.

Another embodiment comprises cyclosporin A, Pluronic L62D®, and Cottonseed oil.

Another embodiment comprises cyclosporin A, Pluronic L62LF®, and Cottonseed oil.

Another embodiment comprises cyclosporin A, Pluronic L64®, and Cottonseed oil.

Another embodiment comprises cyclosporin A, Pluronic L81®, and Cottonseed oil.

Another embodiment comprises cyclosporin A, Pluronic L92®, and Cottonseed oil.

Another embodiment comprises cyclosporin A, Pluronic P103®, and Cottonseed oil.

Another embodiment comprises cyclosporin A, Pluronic P104®, and Cottonseed oil.

Another embodiment comprises cyclosporin A, Pluronic P105®, and Cottonseed oil.

Another embodiment comprises cyclosporin A, Pluronic P123®, and Cottonseed oil.

Another embodiment comprises cyclosporin A, Pluronic P65®, and Cottonseed oil.

Another embodiment comprises cyclosporin A, Pluronic P84®, and Cottonseed oil.

Another embodiment comprises cyclosporin A, Pluronic P85®, and Cottonseed oil.

Another embodiment comprises cyclosporin A, Polyaldo 10-2-P®, and Cottonseed oil.

Another embodiment comprises cyclosporin A, Polyaldo DGDO®, and Cottonseed oil.

Another embodiment comprises cyclosporin A, Polyaldo HGDS®, and Cottonseed oil.

Another embodiment comprises cyclosporin A, Polyaldo TGMS®, and Cottonseed oil.

Another embodiment comprises cyclosporin A, Protegin V®, and Cottonseed oil.

Another embodiment comprises cyclosporin A, Protegin W®, and Cottonseed oil.

Another embodiment comprises cyclosporin A, Protegin WX®, and Cottonseed oil.

Another embodiment comprises cyclosporin A, Protegin XV®, and Cottonseed oil.

Another embodiment comprises cyclosporin A, Rhodasurf BC-420®, and Cottonseed oil.

Another embodiment comprises cyclosporin A, Rhodasurf BC-610®, and Cottonseed oil.

Another embodiment comprises cyclosporin A, Rhodasurf BC-630®, and Cottonseed oil.

Another embodiment comprises cyclosporin A, Rhodasurf BC-720®, and Cottonseed oil.

Another embodiment comprises cyclosporin A, Rhodasurf BC-840®, and Cottonseed oil.

Another embodiment comprises cyclosporin A, Rhodasurf L 1®, and Cottonseed oil.

Another embodiment comprises cyclosporin A, Rhodasurf L 12®, and Cottonseed oil.

Another embodiment comprises cyclosporin A, Rhodasurf L 2®, and Cottonseed oil.

Another embodiment comprises cyclosporin A, Rhodasurf L 20®, and Cottonseed oil.

Another embodiment comprises cyclosporin A, Rhodasurf L 3®, and Cottonseed oil.

Another embodiment comprises cyclosporin A, Rhodasurf L 30®, and Cottonseed oil.

Another embodiment comprises cyclosporin A, Rhodasurf L 4®, and Cottonseed oil.

Another embodiment comprises cyclosporin A, Rhodasurf L 50®, and Cottonseed oil.

Another embodiment comprises cyclosporin A, Rhodasurf L 7/90®, and Cottonseed oil.

Another embodiment comprises cyclosporin A, Rhodasurf LA-3®, and Cottonseed oil.

Another embodiment comprises cyclosporin A, Rhodasurf LA-7®, and Cottonseed oil.

Another embodiment comprises cyclosporin A, Rhodasurf LAN-23®, and Cottonseed oil.

Another embodiment comprises cyclosporin A, Rhodasurf ON-870®, and Cottonseed oil.

Another embodiment comprises cyclosporin A, Rhodasurf ON-877®, and Cottonseed oil.

Another embodiment comprises cyclosporin A, Rhodasurf TDA-8/5®, and Cottonseed oil.

Another embodiment comprises cyclosporin A, Ritapro 100®, and Cottonseed oil.

Another embodiment comprises cyclosporin A, Ritapro 165®, and Cottonseed oil.

Another embodiment comprises cyclosporin A, Ritapro 200®, and Cottonseed oil.

Another embodiment comprises cyclosporin A, Ritapro 300®, and Cottonseed oil.

Another embodiment comprises cyclosporin A, Ritoleth 10®, and Cottonseed oil.

Another embodiment comprises cyclosporin A, Ritoleth 2®, and Cottonseed oil.

Another embodiment comprises cyclosporin A, Ritoleth 20®, and Cottonseed oil.

Another embodiment comprises cyclosporin A, Ritoleth 5®, and Cottonseed oil.

Another embodiment comprises cyclosporin A, Santone 10-10-0®, and Cottonseed oil.

Another embodiment comprises cyclosporin A, Santone 3-1-S XTR®, and Cottonseed oil.

Another embodiment comprises cyclosporin A, Santone 3-1-SH®, and Cottonseed oil.

Another embodiment comprises cyclosporin A, Santone 8-1-O®, and Cottonseed oil.

Another embodiment comprises cyclosporin A, Solulan 16®, and Cottonseed oil.

Another embodiment comprises cyclosporin A, Solulan 75®, and Cottonseed oil.

Another embodiment comprises cyclosporin A, Solulan C-24®, and Cottonseed oil.

Another embodiment comprises cyclosporin A, Solulan L-575®, and Cottonseed oil.

Another embodiment comprises cyclosporin A, Standamox CAW®, and Cottonseed oil.

Another embodiment comprises cyclosporin A, Surfine AZI-A®, and Cottonseed oil.

Another embodiment comprises cyclosporin A, Surfonic CO-15®, and Cottonseed oil.

Another embodiment comprises cyclosporin A, Surfonic CO-25®, and Cottonseed oil.

Another embodiment comprises cyclosporin A, Surfonic CO-30®, and Cottonseed oil.

Another embodiment comprises cyclosporin A, Surfonic CO-36®, and Cottonseed oil.

Another embodiment comprises cyclosporin A, Surfonic CO-42®, and Cottonseed oil.

Another embodiment comprises cyclosporin A, Surfonic E400-MO®, and Cottonseed oil.

Another embodiment comprises cyclosporin A, Surfonic L12-3®, and Cottonseed oil.

Another embodiment comprises cyclosporin A, Surfonic L12-6®, and Cottonseed oil.

Another embodiment comprises cyclosporin A, Surfonic L12-8®, and Cottonseed oil.

Another embodiment comprises cyclosporin A, Surfonic L24-12®, and Cottonseed oil.

Another embodiment comprises cyclosporin A, Surfonic L24-2®, and Cottonseed oil.

Another embodiment comprises cyclosporin A, Surfonic L24-22®, and Cottonseed oil.

Another embodiment comprises cyclosporin A, Surfonic L24-3®, and Cottonseed oil.

Another embodiment comprises cyclosporin A, Surfonic L24-4®, and Cottonseed oil.

Another embodiment comprises cyclosporin A, Surfonic L24-5®, and Cottonseed oil.

Another embodiment comprises cyclosporin A, Surfonic L24-7®, and Cottonseed oil.

Another embodiment comprises cyclosporin A, Surfonic L24-7.1®, and Cottonseed oil.

Another embodiment comprises cyclosporin A, Surfonic L24-9®, and Cottonseed oil.

Another embodiment comprises cyclosporin A, Surfonic LF-17®, and Cottonseed oil.

Another embodiment comprises cyclosporin A, Surfonic LF-18®, and Cottonseed oil.

Another embodiment comprises cyclosporin A, Surfonic LF-37®, and Cottonseed oil.

Another embodiment comprises cyclosporin A, Surfonic LF-41®, and Cottonseed oil.

Another embodiment comprises cyclosporin A, Surfonic LF-42®, and Cottonseed oil.

Another embodiment comprises cyclosporin A, Surfonic LF-47®, and Cottonseed oil.

Another embodiment comprises cyclosporin A, Surfonic LF-68®, and Cottonseed oil.

Another embodiment comprises cyclosporin A, Surfonic N-10®, and Cottonseed oil.

Another embodiment comprises cyclosporin A, Surfonic N-100®, and Cottonseed oil.

Another embodiment comprises cyclosporin A, Surfonic N-102®, and Cottonseed oil.

Another embodiment comprises cyclosporin A, Surfonic N-120®, and Cottonseed oil.

Another embodiment comprises cyclosporin A, Surfonic N-150®, and Cottonseed oil.

Another embodiment comprises cyclosporin A, Surfonic N-200®, and Cottonseed oil.

Another embodiment comprises cyclosporin A, Surfonic N-300®, and Cottonseed oil.

Another embodiment comprises cyclosporin A, Surfonic N-31.5®, and Cottonseed oil.

Another embodiment comprises cyclosporin A, Surfonic N-40®, and Cottonseed oil.

Another embodiment comprises cyclosporin A, Surfonic N-400®, and Cottonseed oil.

Another embodiment comprises cyclosporin A, Surfonic N-60®, and Cottonseed oil.

Another embodiment comprises cyclosporin A, Surfonic N-85®, and Cottonseed oil.

Another embodiment comprises cyclosporin A, Surfonic N-95®, and Cottonseed oil.

Another embodiment comprises cyclosporin A, Surfonic NB-18®, and Cottonseed oil.

Another embodiment comprises cyclosporin A, Surfonic NB-23®, and Cottonseed oil.

Another embodiment comprises cyclosporin A, Surfonic NB-25®, and Cottonseed oil.

Another embodiment comprises cyclosporin A, Surfonic NB-307®, and Cottonseed oil.

Another embodiment comprises cyclosporin A, Surfonic NB-31®, and Cottonseed oil.

Another embodiment comprises cyclosporin A, Surfonic NB-41®, and Cottonseed oil.

Another embodiment comprises cyclosporin A, Surfonic NB-43®, and Cottonseed oil.

Another embodiment comprises cyclosporin A, Surfonic OP-10®, and Cottonseed oil.

Another embodiment comprises cyclosporin A, Surfonic OP-120®, and Cottonseed oil.

Another embodiment comprises cyclosporin A, Surfonic OP-15®, and Cottonseed oil.

Another embodiment comprises cyclosporin A, Surfonic OP-35®, and Cottonseed oil.

Another embodiment comprises cyclosporin A, Surfonic OP-70®, and Cottonseed oil.

Another embodiment comprises cyclosporin A, Surfonic P1®, and Cottonseed oil.

Another embodiment comprises cyclosporin A, Surfonic P3®, and Cottonseed oil.

Another embodiment comprises cyclosporin A, Surfonic P5®, and Cottonseed oil.

Another embodiment comprises cyclosporin A, Surfonic P6®, and Cottonseed oil.

Another embodiment comprises cyclosporin A, Surfonic POA-17R2®, and Cottonseed oil.

Another embodiment comprises cyclosporin A, Surfonic POA-25R2®, and Cottonseed oil.

Another embodiment comprises cyclosporin A, Surfonic TDA-11®, and Cottonseed oil.

Another embodiment comprises cyclosporin A, Surfonic TDA-3B®, and Cottonseed oil.

Another embodiment comprises cyclosporin A, Surfonic TDA-6®, and Cottonseed oil.

Another embodiment comprises cyclosporin A, Surfonic TDA-6/88®, and Cottonseed oil.

Another embodiment comprises cyclosporin A, Surfonic TDA-8®, and Cottonseed oil.

Another embodiment comprises cyclosporin A, Surfonic TDA-8/90®, and Cottonseed oil.

Another embodiment comprises cyclosporin A, Surfonic TDA-9®, and Cottonseed oil.

Another embodiment comprises cyclosporin A, Surfonic TX-CCN®, and Cottonseed oil.

Another embodiment comprises cyclosporin A, Tagat CH 40®, and Cottonseed oil.

Another embodiment comprises cyclosporin A, Tagat CH 60®, and Cottonseed oil.

Another embodiment comprises cyclosporin A, Tagat L 2®, and Cottonseed oil.

Another embodiment comprises cyclosporin A, Tagat S®, and Cottonseed oil.

Another embodiment comprises cyclosporin A, Tagat S 2®, and Cottonseed oil.

Another embodiment comprises cyclosporin A, Tally 100 Plus®, and Cottonseed oil.

Another embodiment comprises cyclosporin A, T-Det A106®, and Cottonseed oil.

Another embodiment comprises cyclosporin A, T-Det A109®, and Cottonseed oil.

Another embodiment comprises cyclosporin A, T-Det A136®, and Cottonseed oil.

Another embodiment comprises cyclosporin A, T-Det A139®, and Cottonseed oil.

Another embodiment comprises cyclosporin A, T-Det A2412®, and Cottonseed oil.

Another embodiment comprises cyclosporin A, T-Det A243®, and Cottonseed oil.

Another embodiment comprises cyclosporin A, T-Det A247®, and Cottonseed oil.

Another embodiment comprises cyclosporin A, T-Det A249®, and Cottonseed oil.

Another embodiment comprises cyclosporin A, T-Det A467®, and Cottonseed oil.

Another embodiment comprises cyclosporin A, T-Det COE®, and Cottonseed oil.

Another embodiment comprises cyclosporin A, T-Det DD-10®, and Cottonseed oil.

Another embodiment comprises cyclosporin A, T-Det DD-5®, and Cottonseed oil.

Another embodiment comprises cyclosporin A, T-Det DD-7®, and Cottonseed oil.

Another embodiment comprises cyclosporin A, T-Det EPO-104®, and Cottonseed oil.

Another embodiment comprises cyclosporin A, T-Det EPO-61®, and Cottonseed oil.

Another embodiment comprises cyclosporin A, T-Det EPO-62®, and Cottonseed oil.

Another embodiment comprises cyclosporin A, T-Det EPO-64®, and Cottonseed oil.

Another embodiment comprises cyclosporin A, T-Det N-1.5®, and Cottonseed oil.

Another embodiment comprises cyclosporin A, T-Det N-10.5®, and Cottonseed oil.

Another embodiment comprises cyclosporin A, T-Det N-100®, and Cottonseed oil.

Another embodiment comprises cyclosporin A, T-Det N-1007®, and Cottonseed oil.

Another embodiment comprises cyclosporin A, T-Det N-12®, and Cottonseed oil.

Another embodiment comprises cyclosporin A, T-Det N-14®, and Cottonseed oil.

Another embodiment comprises cyclosporin A, T-Det N-20®, and Cottonseed oil.

Another embodiment comprises cyclosporin A, T-Det N-30®, and Cottonseed oil.

Another embodiment comprises cyclosporin A, T-Det N-307®, and Cottonseed oil.

Another embodiment comprises cyclosporin A, T-Det N-4®, and Cottonseed oil.

Another embodiment comprises cyclosporin A, T-Det N-40®, and Cottonseed oil.

Another embodiment comprises cyclosporin A, T-Det N-407®, and Cottonseed oil.

Another embodiment comprises cyclosporin A, T-Det N-50®, and Cottonseed oil.

Another embodiment comprises cyclosporin A, T-Det N-507®, and Cottonseed oil.

Another embodiment comprises cyclosporin A, T-Det N-6®, and Cottonseed oil.

Another embodiment comprises cyclosporin A, T-Det N-70®, and Cottonseed oil.

Another embodiment comprises cyclosporin A, T-Det N-8®, and Cottonseed oil.

Another embodiment comprises cyclosporin A, T-Det N-9.5®, and Cottonseed oil.

Another embodiment comprises cyclosporin A, T-Det O-12®, and Cottonseed oil.

Another embodiment comprises cyclosporin A, T-Det O-165®, and Cottonseed oil.

Another embodiment comprises cyclosporin A, T-Det O-307®, and Cottonseed oil.

Another embodiment comprises cyclosporin A, T-Det O-40®, and Cottonseed oil.

Another embodiment comprises cyclosporin A, T-Det O-407®, and Cottonseed oil.

Another embodiment comprises cyclosporin A, T-Det O-6®, and Cottonseed oil.

Another embodiment comprises cyclosporin A, T-Det O-8®, and Cottonseed oil.

Another embodiment comprises cyclosporin A, T-Det O-9®, and Cottonseed oil.

Another embodiment comprises cyclosporin A, Tegin®, and Cottonseed oil.

Another embodiment comprises cyclosporin A, Tergitol 15-S-12®, and Cottonseed oil.

Another embodiment comprises cyclosporin A, Tergitol 15-S-15®, and Cottonseed oil.

Another embodiment comprises cyclosporin A, Tergitol 15-S-20®, and Cottonseed oil.

Another embodiment comprises cyclosporin A, Tergitol 15-S-20 (80AQ)®, and Cottonseed oil.

Another embodiment comprises cyclosporin A, Tergitol 15-S-3®, and Cottonseed oil.

Another embodiment comprises cyclosporin A, Tergitol 15-S-30®, and Cottonseed oil.

Another embodiment comprises cyclosporin A, Tergitol 15-S-40®, and Cottonseed oil.

Another embodiment comprises cyclosporin A, Tergitol 15-S-40 (70AM)®, and Cottonseed oil.

Another embodiment comprises cyclosporin A, Tergitol 15-S-5®, and Cottonseed oil.

Another embodiment comprises cyclosporin A, Tergitol 15-S-7®, and Cottonseed oil.

Another embodiment comprises cyclosporin A, Tergitol 15-S-9®, and Cottonseed oil.

Another embodiment comprises cyclosporin A, Tergitol L-101®, and Cottonseed oil.

Another embodiment comprises cyclosporin A, Tergitol L-61®, and Cottonseed oil.

Another embodiment comprises cyclosporin A, Tergitol L-62®, and Cottonseed oil.

Another embodiment comprises cyclosporin A, Tergitol L-64®, and Cottonseed oil.

Another embodiment comprises cyclosporin A, Tergitol L-81®, and Cottonseed oil.

Another embodiment comprises cyclosporin A, Tergitol NP-10®, and Cottonseed oil.

Another embodiment comprises cyclosporin A, Tergitol NP-11®, and Cottonseed oil.

Another embodiment comprises cyclosporin A, Tergitol NP-12®, and Cottonseed oil.

Another embodiment comprises cyclosporin A, Tergitol NP-13®, and Cottonseed oil.

Another embodiment comprises cyclosporin A, Tergitol NP-15®, and Cottonseed oil.

Another embodiment comprises cyclosporin A, Tergitol NP-30®, and Cottonseed oil.

Another embodiment comprises cyclosporin A, Tergitol NP-30 (70 AQ)®, and Cottonseed oil.

Another embodiment comprises cyclosporin A, Tergitol NP-4®, and Cottonseed oil.

Another embodiment comprises cyclosporin A, Tergitol NP-40®, and Cottonseed oil.

Another embodiment comprises cyclosporin A, Tergitol NP-40 (70 AQ)®, and Cottonseed oil.

Another embodiment comprises cyclosporin A, Tergitol NP-50 (70 AQ)®, and Cottonseed oil.

Another embodiment comprises cyclosporin A, Tergitol NP-55 (70 AQ)®, and Cottonseed oil.

Another embodiment comprises cyclosporin A, Tergitol NP-6®, and Cottonseed oil.

Another embodiment comprises cyclosporin A, Tergitol NP-7®, and Cottonseed oil.

Another embodiment comprises cyclosporin A, Tergitol NP-70 (70 AQ)®, and Cottonseed oil.

Another embodiment comprises cyclosporin A, Tergitol NP-8®, and Cottonseed oil.

Another embodiment comprises cyclosporin A, Tergitol NP-9®, and Cottonseed oil.

Another embodiment comprises cyclosporin A, Tergitol NP-9.5®, and Cottonseed oil.

Another embodiment comprises cyclosporin A, Tergitol TMN-10®, and Cottonseed oil.

Another embodiment comprises cyclosporin A, Tergitol TMN-3®, and Cottonseed oil.

Another embodiment comprises cyclosporin A, Tergitol TMN-6®, and Cottonseed oil.

Another embodiment comprises cyclosporin A, Tergitol XD®, and Cottonseed oil.

Another embodiment comprises cyclosporin A, Tergitol XH®, and Cottonseed oil.

Another embodiment comprises cyclosporin A, Tergitol XJ®, and Cottonseed oil.

Another embodiment comprises cyclosporin A, Tetronic 1107®, and Cottonseed oil.

Another embodiment comprises cyclosporin A, Tetronic 1307®, and Cottonseed oil.

Another embodiment comprises cyclosporin A, Tetronic 150R1®, and Cottonseed oil.

Another embodiment comprises cyclosporin A, Tetronic 304®, and Cottonseed oil.

Another embodiment comprises cyclosporin A, Tetronic 701®, and Cottonseed oil.

Another embodiment comprises cyclosporin A, Tetronic 901®, and Cottonseed oil.

Another embodiment comprises cyclosporin A, Tetronic 904®, and Cottonseed oil.

Another embodiment comprises cyclosporin A, Tetronic 908®, and Cottonseed oil.

Another embodiment comprises cyclosporin A, Tetronic 90R4®, and Cottonseed oil.

Another embodiment comprises cyclosporin A, Tomadol 1-3®, and Cottonseed oil.

Another embodiment comprises cyclosporin A, Tomadol 1-5®, and Cottonseed oil.

Another embodiment comprises cyclosporin A, Tomadol 1-7®, and Cottonseed oil.

Another embodiment comprises cyclosporin A, Tomadol 23-1®, and Cottonseed oil.

Another embodiment comprises cyclosporin A, Tomadol 23-3®, and Cottonseed oil.

Another embodiment comprises cyclosporin A, Tomadol 23-5®, and Cottonseed oil.

Another embodiment comprises cyclosporin A, Tomadol 23-6.5®, and Cottonseed oil.

Another embodiment comprises cyclosporin A, Tomadol 25-12®, and Cottonseed oil.

Another embodiment comprises cyclosporin A, Tomadol 25-3®, and Cottonseed oil.

Another embodiment comprises cyclosporin A, Tomadol 25-7®, and Cottonseed oil.

Another embodiment comprises cyclosporin A, Tomadol 25-9®, and Cottonseed oil.

Another embodiment comprises cyclosporin A, Tomadol 45-13®, and Cottonseed oil.

Another embodiment comprises cyclosporin A, Tomadol 45-2.5®, and Cottonseed oil.

Another embodiment comprises cyclosporin A, Tomadol 45-7®, and Cottonseed oil.

Another embodiment comprises cyclosporin A, Tomadol 91-2.5®, and Cottonseed oil.

Another embodiment comprises cyclosporin A, Tomadol 91-6®, and Cottonseed oil.

Another embodiment comprises cyclosporin A, Tomadol 91-8®, and Cottonseed oil.

Another embodiment comprises cyclosporin A, Triton X-100®, and Cottonseed oil.

Another embodiment comprises cyclosporin A, Triton X-102®, and Cottonseed oil.

Another embodiment comprises cyclosporin A, Triton X-114®, and Cottonseed oil.

Another embodiment comprises cyclosporin A, Triton X-15®, and Cottonseed oil.

Another embodiment comprises cyclosporin A, Triton X-165 (70%)®, and Cottonseed oil.

Another embodiment comprises cyclosporin A, Triton X-207®, and Cottonseed oil.

Another embodiment comprises cyclosporin A, Triton X-305 (70%)®, and Cottonseed oil.

Another embodiment comprises cyclosporin A, Triton X-35®, and Cottonseed oil.

Another embodiment comprises cyclosporin A, Triton X-405 (70%)®, and Cottonseed oil.

Another embodiment comprises cyclosporin A, Triton X-45®, and Cottonseed oil.

Another embodiment comprises cyclosporin A, Triton X-705 (70%)®, and Cottonseed oil.

Another embodiment comprises cyclosporin A, Volpo S-10®, and Cottonseed oil.

Another embodiment comprises cyclosporin A, Volpo S-2®, and Cottonseed oil.

Another embodiment comprises cyclosporin A, Volpo S-20®, and Cottonseed oil.

Another embodiment comprises cyclosporin A, Volpo-10®, and Cottonseed oil.

Another embodiment comprises cyclosporin A, Volpo-20®, and Cottonseed oil.

Another embodiment comprises cyclosporin A, Volpo-3®, and Cottonseed oil.

Another embodiment comprises cyclosporin A, Volpo-5®, and Cottonseed oil.

Another embodiment comprises cyclosporin A, Abex 2515®, and Safflower oil.

Another embodiment comprises cyclosporin A, Abex 2525/40®, and Safflower oil.

Another embodiment comprises cyclosporin A, Abex 2535®, and Safflower oil.

Another embodiment comprises cyclosporin A, Abex 2545®, and Safflower oil.

Another embodiment comprises cyclosporin A, Acconon E®, and Safflower oil.

Another embodiment comprises cyclosporin A, Alfonic 1216CO-9®, and Safflower oil.

Another embodiment comprises cyclosporin A, Alfonic 1412-3®, and Safflower oil.

Another embodiment comprises cyclosporin A, Alfonic TDA-12®, and Safflower oil.

Another embodiment comprises cyclosporin A, Alfonic TDA-3®, and Safflower oil.

Another embodiment comprises cyclosporin A, Alfonic TDA-4®, and Safflower oil.

Another embodiment comprises cyclosporin A, Alfonic TDA-6®, and Safflower oil.

Another embodiment comprises cyclosporin A, Alfonic TDA-7®, and Safflower oil.

Another embodiment comprises cyclosporin A, Alfonic TDA-8®, and Safflower oil.

Another embodiment comprises cyclosporin A, Alfonic TDA-9®, and Safflower oil.

Another embodiment comprises cyclosporin A, Alkamuls 400-DO®, and Safflower oil.

Another embodiment comprises cyclosporin A, Alkamuls 400-MO/E®, and Safflower oil.

Another embodiment comprises cyclosporin A, Ameroxol OE-10®, and Safflower oil.

Another embodiment comprises cyclosporin A, Ameroxol OE-2®, and Safflower oil.

Another embodiment comprises cyclosporin A, Ameroxol OE-20®, and Safflower oil.

Another embodiment comprises cyclosporin A, Armix 176®, and Safflower oil.

Another embodiment comprises cyclosporin A, Armix 180-C®, and Safflower oil.

Another embodiment comprises cyclosporin A, Armix 183®, and Safflower oil.

Another embodiment comprises cyclosporin A, Brij 35®, and Safflower oil.

Another embodiment comprises cyclosporin A, Brij 35 Liquid®, and Safflower oil.

Another embodiment comprises cyclosporin A, Brij 35 SP®, and Safflower oil.

Another embodiment comprises cyclosporin A, Canasol MJ 2109®, and Safflower oil.

Another embodiment comprises cyclosporin A, Canasol MJ 52®, and Safflower oil.

Another embodiment comprises cyclosporin A, Canasol R 3600®, and Safflower oil.

Another embodiment comprises cyclosporin A, Canasol R 3603®, and Safflower oil.

Another embodiment comprises cyclosporin A, Canasol R 4000 H®, and Safflower oil.

Another embodiment comprises cyclosporin A, Cedepal CA-210®, and Safflower oil.

Another embodiment comprises cyclosporin A, Cedepal CA-520®, and Safflower oil.

Another embodiment comprises cyclosporin A, Cedepal CA-630®, and Safflower oil.

Another embodiment comprises cyclosporin A, Cedepal CA-720®, and Safflower oil.

Another embodiment comprises cyclosporin A, Cedepal CA-890®, and Safflower oil.

Another embodiment comprises cyclosporin A, Cedepal CA-897®, and Safflower oil.

Another embodiment comprises cyclosporin A, Cedepal CO-210®, and Safflower oil.

Another embodiment comprises cyclosporin A, Cedepal CO-430®, and Safflower oil.

Another embodiment comprises cyclosporin A, Cedepal CO-530®, and Safflower oil.

Another embodiment comprises cyclosporin A, Cedepal CO-630®, and Safflower oil.

Another embodiment comprises cyclosporin A, Cedepal CO-710®, and Safflower oil.

Another embodiment comprises cyclosporin A, Cedepal CO-730®, and Safflower oil.

Another embodiment comprises cyclosporin A, Cedepal CO-880®, and Safflower oil.

Another embodiment comprises cyclosporin A, Cedepal CO-887®, and Safflower oil.

Another embodiment comprises cyclosporin A, Cedepal CO-890®, and Safflower oil.

Another embodiment comprises cyclosporin A, Cedepal CO-897®, and Safflower oil.

Another embodiment comprises cyclosporin A, Cedepal CO-970®, and Safflower oil.

Another embodiment comprises cyclosporin A, Cedepal CO-977®, and Safflower oil.

Another embodiment comprises cyclosporin A, Cedepal CO-990®, and Safflower oil.

Another embodiment comprises cyclosporin A, Cedepal CO-997®, and Safflower oil.

Another embodiment comprises cyclosporin A, Chemal BP 261®, and Safflower oil.

Another embodiment comprises cyclosporin A, Chemal BP 262®, and Safflower oil.

Another embodiment comprises cyclosporin A, Chemal BP 3172®, and Safflower oil.

Another embodiment comprises cyclosporin A, Chemal BP 3174®, and Safflower oil.

Another embodiment comprises cyclosporin A, Chemal DA-4®, and Safflower oil.

Another embodiment comprises cyclosporin A, Chemal DA-6®, and Safflower oil.

Another embodiment comprises cyclosporin A, Chemal DA-9®, and Safflower oil.

Another embodiment comprises cyclosporin A, Chemal LA-23®, and Safflower oil.

Another embodiment comprises cyclosporin A, Chemal LA-4®, and Safflower oil.

Another embodiment comprises cyclosporin A, Chemal LA-9®, and Safflower oil.

Another embodiment comprises cyclosporin A, Chemal OA-20G®, and Safflower oil.

Another embodiment comprises cyclosporin A, Chemal OA-5®, and Safflower oil.

Another embodiment comprises cyclosporin A, Chemal OA-9®, and Safflower oil.

Another embodiment comprises cyclosporin A, Chemal TDA-12®, and Safflower oil.

Another embodiment comprises cyclosporin A, Chemal TDA-15®, and Safflower oil.

Another embodiment comprises cyclosporin A, Chemal TDA-3®, and Safflower oil.

Another embodiment comprises cyclosporin A, Chemal TDA-6®, and Safflower oil.

Another embodiment comprises cyclosporin A, Chemal TDA-9®, and Safflower oil.

Another embodiment comprises cyclosporin A, Chemax CO-16®, and Safflower oil.

Another embodiment comprises cyclosporin A, Chemax CO-200/50®, and Safflower oil.

Another embodiment comprises cyclosporin A, Chemax CO-25®, and Safflower oil.

Another embodiment comprises cyclosporin A, Chemax CO-30®, and Safflower oil.

Another embodiment comprises cyclosporin A, Chemax CO-36®, and Safflower oil.

Another embodiment comprises cyclosporin A, Chemax CO-40®, and Safflower oil.

Another embodiment comprises cyclosporin A, Chemax CO-5®, and Safflower oil.

Another embodiment comprises cyclosporin A, Chemax CO-80®, and Safflower oil.

Another embodiment comprises cyclosporin A, Chemax DNP-150/50®, and Safflower oil.

Another embodiment comprises cyclosporin A, Chemax DN P-18®, and Safflower oil.

Another embodiment comprises cyclosporin A, Chemax DNP-8®, and Safflower oil.

Another embodiment comprises cyclosporin A, Chemax E 200 ML®, and Safflower oil.

Another embodiment comprises cyclosporin A, Chemax E 200 MO®, and Safflower oil.

Another embodiment comprises cyclosporin A, Chemax E 400 ML®, and Safflower oil.

Another embodiment comprises cyclosporin A, Chemax E 400 MO®, and Safflower oil.

Another embodiment comprises cyclosporin A, Chemax E 600 ML®, and Safflower oil.

Another embodiment comprises cyclosporin A, Chemax E 600 MO®, and Safflower oil.

Another embodiment comprises cyclosporin A, Chemax E1000 MO®, and Safflower oil.

Another embodiment comprises cyclosporin A, Chemax E1000 MS®, and Safflower oil.

Another embodiment comprises cyclosporin A, Chemax E200 MS®, and Safflower oil.

Another embodiment comprises cyclosporin A, Chemax E400 MS®, and Safflower oil.

Another embodiment comprises cyclosporin A, Chemax E600 MS®, and Safflower oil.

Another embodiment comprises cyclosporin A, Chemax HCO-16®, and Safflower oil.

Another embodiment comprises cyclosporin A, Chemax HCO-200/50®, and Safflower oil.

Another embodiment comprises cyclosporin A, Chemax HCO-25®, and Safflower oil.

Another embodiment comprises cyclosporin A, Chemax HCO-5®, and Safflower oil.

Another embodiment comprises cyclosporin A, Chemax TO-16®, and Safflower oil.

Another embodiment comprises cyclosporin A, Chemax TO-8®, and Safflower oil.

Another embodiment comprises cyclosporin A, Colalube 3409®, and Safflower oil.

Another embodiment comprises cyclosporin A, Colalube 3411®, and Safflower oil.

Another embodiment comprises cyclosporin A, Colalube 3413®, and Safflower oil.

Another embodiment comprises cyclosporin A, Colalube 3414®, and Safflower oil.

Another embodiment comprises cyclosporin A, Colalube 3415®, and Safflower oil.

Another embodiment comprises cyclosporin A, Colalube 3416®, and Safflower oil.

Another embodiment comprises cyclosporin A, Colalube 3417®, and Safflower oil.

Another embodiment comprises cyclosporin A, Delonic NPE-100®, and Safflower oil.

Another embodiment comprises cyclosporin A, Delonic NPE-30®, and Safflower oil.

Another embodiment comprises cyclosporin A, Delonic NPE-40®, and Safflower oil.

Another embodiment comprises cyclosporin A, Delonic OPE-10®, and Safflower oil.

Another embodiment comprises cyclosporin A, Delonic OPE-12®, and Safflower oil.

Another embodiment comprises cyclosporin A, Delonic OPE-30®, and Safflower oil.

Another embodiment comprises cyclosporin A, Delonic OPE-40®, and Safflower oil.

Another embodiment comprises cyclosporin A, Delonic OPE-5®, and Safflower oil.

Another embodiment comprises cyclosporin A, Delonic OPE-7.5®, and Safflower oil.

Another embodiment comprises cyclosporin A, DePeg 16-CO®, and Safflower oil.

Another embodiment comprises cyclosporin A, DePeg 25-CO®, and Safflower oil.

Another embodiment comprises cyclosporin A, DePeg 30-CO®, and Safflower oil.

Another embodiment comprises cyclosporin A, DePeg 40-CO®, and Safflower oil.

Another embodiment comprises cyclosporin A, DePeg 5-CO®, and Safflower oil.

Another embodiment comprises cyclosporin A, DePeg 80-CO®, and Safflower oil.

Another embodiment comprises cyclosporin A, DeSonic S-10®, and Safflower oil.

Another embodiment comprises cyclosporin A, DeSonic S-114®, and Safflower oil.

Another embodiment comprises cyclosporin A, DeSonic S-405®, and Safflower oil.

Another embodiment comprises cyclosporin A, DeSonic S-45®, and Safflower oil.

Another embodiment comprises cyclosporin A, DeSonic TDA-9®, and Safflower oil.

Another embodiment comprises cyclosporin A, DeThox Acid O-14®, and Safflower oil.

Another embodiment comprises cyclosporin A, DeThox Acid O-16®, and Safflower oil.

Another embodiment comprises cyclosporin A, DeThox Acid O-9®, and Safflower oil.

Another embodiment comprises cyclosporin A, DeThox Acid TO-14®, and Safflower oil.

Another embodiment comprises cyclosporin A, DeThox Acid TO-16.5®, and Safflower oil.

Another embodiment comprises cyclosporin A, DeThox Acid TO-8.5®, and Safflower oil.

Another embodiment comprises cyclosporin A, Dur-Em 114®, and Safflower oil.

Another embodiment comprises cyclosporin A, Dur-Em 117, and Safflower oil.

Another embodiment comprises cyclosporin A, Dur-Em 204®, and Safflower oil.

Another embodiment comprises cyclosporin A, Dur-Em 207®, and Safflower oil.

Another embodiment comprises cyclosporin A, Dur-Em 207E®, and Safflower oil.

Another embodiment comprises cyclosporin A, Emerest 2704®, and Safflower oil.

Another embodiment comprises cyclosporin A, Emerest 2712®, and Safflower oil.

Another embodiment comprises cyclosporin A, Emerest 2715®, and Safflower oil.

Another embodiment comprises cyclosporin A, Ethal EH-2®, and Safflower oil.

Another embodiment comprises cyclosporin A, Ethal EH-5®, and Safflower oil.

Another embodiment comprises cyclosporin A, Ethal OA-23®, and Safflower oil.

Another embodiment comprises cyclosporin A, Ethosperse CA-20®, and Safflower oil.

Another embodiment comprises cyclosporin A, Ethosperse LA-12®, and Safflower oil.

Another embodiment comprises cyclosporin A, Ethosperse LA-23®, and Safflower oil.

Another embodiment comprises cyclosporin A, Ethosperse LA-4®, and Safflower oil.

Another embodiment comprises cyclosporin A, Ethox 3095®, and Safflower oil.

Another embodiment comprises cyclosporin A, Ethox CO-16®, and Safflower oil.

Another embodiment comprises cyclosporin A, Ethox CO-200®, and Safflower oil.

Another embodiment comprises cyclosporin A, Ethox CO-25®, and Safflower oil.

Another embodiment comprises cyclosporin A, Ethox CO-30®, and Safflower oil.

Another embodiment comprises cyclosporin A, Ethox CO-36®, and Safflower oil.

Another embodiment comprises cyclosporin A, Ethox CO-40®, and Safflower oil.

Another embodiment comprises cyclosporin A, Ethox CO-5®, and Safflower oil.

Another embodiment comprises cyclosporin A, Ethox DO-14®, and Safflower oil.

Another embodiment comprises cyclosporin A, Ethox DO-9®, and Safflower oil.

Another embodiment comprises cyclosporin A, Ethox MA-8®, and Safflower oil.

Another embodiment comprises cyclosporin A, Ethox MI-14®, and Safflower oil.

Another embodiment comprises cyclosporin A, Ethox ML-14®, and Safflower oil.

Another embodiment comprises cyclosporin A, Ethox ML-5®, and Safflower oil.

Another embodiment comprises cyclosporin A, Ethox MO-14®, and Safflower oil.

Another embodiment comprises cyclosporin A, Ethox MO-9®, and Safflower oil.

Another embodiment comprises cyclosporin A, Ethox MS-14®, and Safflower oil.

Another embodiment comprises cyclosporin A, Ethox MS-23®, and Safflower oil.

Another embodiment comprises cyclosporin A, Ethox MS-40®, and Safflower oil.

Another embodiment comprises cyclosporin A, Ethox MS-8®, and Safflower oil.

Another embodiment comprises cyclosporin A, Ethox TO-16®, and Safflower oil.

Another embodiment comprises cyclosporin A, Ethox TO-8®, and Safflower oil.

Another embodiment comprises cyclosporin A, Ethylan SN-12®, and Safflower oil.

Another embodiment comprises cyclosporin A, Ethylan SN-70®, and Safflower oil.

Another embodiment comprises cyclosporin A, Ethylan SN-90®, and Safflower oil.

Another embodiment comprises cyclosporin A, Flexricin 13®, and Safflower oil.

Another embodiment comprises cyclosporin A, Flexricin 15®, and Safflower oil.

Another embodiment comprises cyclosporin A, Genapol 1261®, and Safflower oil.

Another embodiment comprises cyclosporin A, Generol 122 E25®, and Safflower oil.

Another embodiment comprises cyclosporin A, Glicepol 160®, and Safflower oil.

Another embodiment comprises cyclosporin A, Glicepol 560®, and Safflower oil.

Another embodiment comprises cyclosporin A, Glicepol GMS 20®, and Safflower oil.

Another embodiment comprises cyclosporin A, Hetoxide BN-13®, and Safflower oil.

Another embodiment comprises cyclosporin A, Hetoxide DNP-4®, and Safflower oil.

Another embodiment comprises cyclosporin A, Hetoxide DNP-9.6®, and Safflower oil.

Another embodiment comprises cyclosporin A, Hetoxide G-26®, and Safflower oil.

Another embodiment comprises cyclosporin A, Hetoxide G-7®, and Safflower oil.

Another embodiment comprises cyclosporin A, Hetoxide GT-20®, and Safflower oil.

Another embodiment comprises cyclosporin A, Hetoxide HC-40®, and Safflower oil.

Another embodiment comprises cyclosporin A, Hetoxol C-24®, and Safflower oil.

Another embodiment comprises cyclosporin A, Hetoxol CA-10®, and Safflower oil.

Another embodiment comprises cyclosporin A, Hetoxol CA-2®, and Safflower oil.

Another embodiment comprises cyclosporin A, Hetoxol CA-20®, and Safflower oil.

Another embodiment comprises cyclosporin A, Hetoxol CAWS®, and Safflower oil.

Another embodiment comprises cyclosporin A, Hetoxol CD-3®, and Safflower oil.

Another embodiment comprises cyclosporin A, Hetoxol CD-4®, and Safflower oil.

Another embodiment comprises cyclosporin A, Hetoxol CS-15®, and Safflower oil.

Another embodiment comprises cyclosporin A, Hetoxol CS-20®, and Safflower oil.

Another embodiment comprises cyclosporin A, Hetoxol CS-20D®, and Safflower oil.

Another embodiment comprises cyclosporin A, Hetoxol CS-25®, and Safflower oil.

Another embodiment comprises cyclosporin A, Hetoxol CS-50®, and Safflower oil.

Another embodiment comprises cyclosporin A, Hetoxol CS-9®, and Safflower oil.

Another embodiment comprises cyclosporin A, Hetoxol CSA-15®, and Safflower oil.

Another embodiment comprises cyclosporin A, Hetoxol D®, and Safflower oil.

Another embodiment comprises cyclosporin A, Hetoxol G®, and Safflower oil.

Another embodiment comprises cyclosporin A, Hetoxol J®, and Safflower oil.

Another embodiment comprises cyclosporin A, Hetoxol L®, and Safflower oil.

Another embodiment comprises cyclosporin A, Hetoxol L-23®, and Safflower oil.

Another embodiment comprises cyclosporin A, Hetoxol L-4®, and Safflower oil.

Another embodiment comprises cyclosporin A, Hetoxol L-9®, and Safflower oil.

Another embodiment comprises cyclosporin A, Hetoxol LS-9®, and Safflower oil.

Another embodiment comprises cyclosporin A, Hetoxol M-3®, and Safflower oil.

Another embodiment comprises cyclosporin A, Hetoxol OA-10 Special®, and Safflower oil.

Another embodiment comprises cyclosporin A, Hetoxol OA-20 Special®, and Safflower oil.

Another embodiment comprises cyclosporin A, Hetoxol OA-3 Special®, and Safflower oil.

Another embodiment comprises cyclosporin A, Hetoxol OA-5 Special®, and Safflower oil.

Another embodiment comprises cyclosporin A, Hetoxol OL-10®, and Safflower oil.

Another embodiment comprises cyclosporin A, Hetoxol OL-2®, and Safflower oil.

Another embodiment comprises cyclosporin A, Hetoxol OL-23®, and Safflower oil.

Another embodiment comprises cyclosporin A, Hetoxol OL-4®, and Safflower oil.

Another embodiment comprises cyclosporin A, Hetoxol OL-40®, and Safflower oil.

Another embodiment comprises cyclosporin A, Hetoxol OL-5®, and Safflower oil.

Another embodiment comprises cyclosporin A, Hetoxol PLA®, and Safflower oil.

Another embodiment comprises cyclosporin A, Hetoxol SP-15®, and Safflower oil.

Another embodiment comprises cyclosporin A, Hetoxol STA-10®, and Safflower oil.

Another embodiment comprises cyclosporin A, Hetoxol STA-2®, and Safflower oil.

Another embodiment comprises cyclosporin A, Hetoxol STA-30®, and Safflower oil.

Another embodiment comprises cyclosporin A, Hetoxol TD-12®, and Safflower oil.

Another embodiment comprises cyclosporin A, Hetoxol TD-18®, and Safflower oil.

Another embodiment comprises cyclosporin A, Hetoxol TD-3®, and Safflower oil.

Another embodiment comprises cyclosporin A, Hetoxol TD-6®, and Safflower oil.

Another embodiment comprises cyclosporin A, Hetoxol TD-9®, and Safflower oil.

Another embodiment comprises cyclosporin A, Hetoxol TDEP-15®, and Safflower oil.

Another embodiment comprises cyclosporin A, Hetoxol TDEP-63®, and Safflower oil.

Another embodiment comprises cyclosporin A, Iconol 24-12®, and Safflower oil.

Another embodiment comprises cyclosporin A, Iconol 24-23®, and Safflower oil.

Another embodiment comprises cyclosporin A, Iconol 24-4®, and Safflower oil.

Another embodiment comprises cyclosporin A, Iconol DA-4®, and Safflower oil.

Another embodiment comprises cyclosporin A, Iconol DA-6®, and Safflower oil.

Another embodiment comprises cyclosporin A, Iconol TDA-10®, and Safflower oil.

Another embodiment comprises cyclosporin A, Iconol TDA-3®, and Safflower oil.

Another embodiment comprises cyclosporin A, Iconol TDA-6®, and Safflower oil.

Another embodiment comprises cyclosporin A, Iconol TDA-8-90%®, and Safflower oil.

Another embodiment comprises cyclosporin A, Iconol TDA-9®, and Safflower oil.

Another embodiment comprises cyclosporin A, Igepal CA-210®, and Safflower oil.

Another embodiment comprises cyclosporin A, Igepal CA-407®, and Safflower oil.

Another embodiment comprises cyclosporin A, Igepal CA-420®, and Safflower oil.

Another embodiment comprises cyclosporin A, Igepal CA-520®, and Safflower oil.

Another embodiment comprises cyclosporin A, Igepal CA-620®, and Safflower oil.

Another embodiment comprises cyclosporin A, Igepal CA-630®, and Safflower oil.

Another embodiment comprises cyclosporin A, Igepal CA-720®, and Safflower oil.

Another embodiment comprises cyclosporin A, Igepal CA-877®, and Safflower oil.

Another embodiment comprises cyclosporin A, Igepal CA-887®, and Safflower oil.

Another embodiment comprises cyclosporin A, Igepal CA-897®, and Safflower oil.

Another embodiment comprises cyclosporin A, Igepal CO-210®, and Safflower oil.

Another embodiment comprises cyclosporin A, Igepal CO-430®, and Safflower oil.

Another embodiment comprises cyclosporin A, Igepal CO-520®, and Safflower oil.

Another embodiment comprises cyclosporin A, Igepal CO-530®, and Safflower oil.

Another embodiment comprises cyclosporin A, Igepal CO-610®, and Safflower oil.

Another embodiment comprises cyclosporin A, Igepal CO-630®, and Safflower oil.

Another embodiment comprises cyclosporin A, Igepal CO-630 Special®, and Safflower oil.

Another embodiment comprises cyclosporin A, Igepal CO-660®, and Safflower oil.

Another embodiment comprises cyclosporin A, Igepal CO-710®, and Safflower oil.

Another embodiment comprises cyclosporin A, Igepal CO-720®, and Safflower oil.

Another embodiment comprises cyclosporin A, Igepal CO-730®, and Safflower oil.

Another embodiment comprises cyclosporin A, Igepal CO-738®, and Safflower oil.

Another embodiment comprises cyclosporin A, Igepal CO-850®, and Safflower oil.

Another embodiment comprises cyclosporin A, Igepal CO-880®, and Safflower oil.

Another embodiment comprises cyclosporin A, Igepal CO-887®, and Safflower oil.

Another embodiment comprises cyclosporin A, Igepal CO-890®, and Safflower oil.

Another embodiment comprises cyclosporin A, Igepal CO-897®, and Safflower oil.

Another embodiment comprises cyclosporin A, Igepal CO-970®, and Safflower oil.

Another embodiment comprises cyclosporin A, Igepal CO-977®, and Safflower oil.

Another embodiment comprises cyclosporin A, Igepal CO-987®, and Safflower oil.

Another embodiment comprises cyclosporin A, Igepal CO-990 FLK®, and Safflower oil.

Another embodiment comprises cyclosporin A, Igepal CO-997®, and Safflower oil.

Another embodiment comprises cyclosporin A, Igepal DM-430®, and Safflower oil.

Another embodiment comprises cyclosporin A, Igepal DM-530®, and Safflower oil.

Another embodiment comprises cyclosporin A, Igepal DM-710®, and Safflower oil.

Another embodiment comprises cyclosporin A, Igepal DM-730®, and Safflower oil.

Another embodiment comprises cyclosporin A, Igepal DM-880®, and Safflower oil.

Another embodiment comprises cyclosporin A, Igepal DM-970 FLK®, and Safflower oil.

Another embodiment comprises cyclosporin A, Igepal OD-410®, and Safflower oil.

Another embodiment comprises cyclosporin A, Igepal RC-520®, and Safflower oil.

Another embodiment comprises cyclosporin A, Igepal RC-620®, and Safflower oil.

Another embodiment comprises cyclosporin A, Igepal RC-630®, and Safflower oil.

Another embodiment comprises cyclosporin A, Lexemul CS-20®, and Safflower oil.

Another embodiment comprises cyclosporin A, Lipo GMS 450®, and Safflower oil.

Another embodiment comprises cyclosporin A, Lipo Polyglycol 1000®, and Safflower oil.

Another embodiment comprises cyclosporin A, Lipo Polyglycol 200®, and Safflower oil.

Another embodiment comprises cyclosporin A, Lipo Polyglycol 300®, and Safflower oil.

Another embodiment comprises cyclosporin A, Lipo Polyglycol 3350®, and Safflower oil.

Another embodiment comprises cyclosporin A, Lipo Polyglycol 400®, and Safflower oil.

Another embodiment comprises cyclosporin A, Lipo Polyglycol 600®, and Safflower oil.

Another embodiment comprises cyclosporin A, Lipocol C-10®, and Safflower oil.

Another embodiment comprises cyclosporin A, Lipocol C-2®, and Safflower oil.

Another embodiment comprises cyclosporin A, Lipocol C-20®, and Safflower oil.

Another embodiment comprises cyclosporin A, Lipocol L-12®, and Safflower oil.

Another embodiment comprises cyclosporin A, Lipocol L-23®, and Safflower oil.

Another embodiment comprises cyclosporin A, Lipocol L-4®, and Safflower oil.

Another embodiment comprises cyclosporin A, Lipocol O-10®, and Safflower oil.

Another embodiment comprises cyclosporin A, Lipocol O-2®, and Safflower oil.

Another embodiment comprises cyclosporin A, Lipocol O-20®, and Safflower oil.

Another embodiment comprises cyclosporin A, Lipocol O-5®, and Safflower oil.

Another embodiment comprises cyclosporin A, Lipocol S-10®, and Safflower oil.

Another embodiment comprises cyclosporin A, Lipocol S-2®, and Safflower oil.

Another embodiment comprises cyclosporin A, Lipocol S-20®, and Safflower oil.

Another embodiment comprises cyclosporin A, Lipocol SC-15®, and Safflower oil.

Another embodiment comprises cyclosporin A, Lipocol SC-20®, and Safflower oil.

Another embodiment comprises cyclosporin A, Lipocol SC-4®, and Safflower oil.

Another embodiment comprises cyclosporin A, Lipocol TD-12®, and Safflower oil.

Another embodiment comprises cyclosporin A, Lumulse 40-O K®, and Safflower oil.

Another embodiment comprises cyclosporin A, Lumulse 42-O K®, and Safflower oil.

Another embodiment comprises cyclosporin A, Lumulse GMT-40®, and Safflower oil.

Another embodiment comprises cyclosporin A, Lumulse PEG 1450®, and Safflower oil.

Another embodiment comprises cyclosporin A, Lumulse PEG 1450 NF®, and Safflower oil.

Another embodiment comprises cyclosporin A, Lumulse PEG 8000®, and Safflower oil.

Another embodiment comprises cyclosporin A, Lumulse PGO®, and Safflower oil.

Another embodiment comprises cyclosporin A, Lumulse POE (2) Oleyl Amine®, and Safflower oil.

Another embodiment comprises cyclosporin A, Magrabar PGO-104®, and Safflower oil.

Another embodiment comprises cyclosporin A, Mapeg 200 ML®, and Safflower oil.

Another embodiment comprises cyclosporin A, Mapeg 400 MO®, and Safflower oil.

Another embodiment comprises cyclosporin A, Mapeg 400 MOT®, and Safflower oil.

Another embodiment comprises cyclosporin A, Mapeg 400DO®, and Safflower oil.

Another embodiment comprises cyclosporin A, Mapeg 400DOT®, and Safflower oil.

Another embodiment comprises cyclosporin A, Mapeg 600DOT®, and Safflower oil.

Another embodiment comprises cyclosporin A, Marlox B 24/80®, and Safflower oil.

Another embodiment comprises cyclosporin A, Marlox FK 86®, and Safflower oil.

Another embodiment comprises cyclosporin A, Marlox MO 154®, and Safflower oil.

Another embodiment comprises cyclosporin A, Mazox CDA CG®, and Safflower oil.

Another embodiment comprises cyclosporin A, Monoglycerides Glyceryl Monestearate®, and Safflower oil.

Another embodiment comprises cyclosporin A, Myrj 45®, and Safflower oil.

Another embodiment comprises cyclosporin A, Myrj 52®, and Safflower oil.

Another embodiment comprises cyclosporin A, Myrj 52S®, and Safflower oil.

Another embodiment comprises cyclosporin A, Myrj 53®, and Safflower oil.

Another embodiment comprises cyclosporin A, Myrj 59®, and Safflower oil.

Another embodiment comprises cyclosporin A, Norfox NP-12®, and Safflower oil.

Another embodiment comprises cyclosporin A, Norfox NP-4®, and Safflower oil.

Another embodiment comprises cyclosporin A, Norfox NP-6®, and Safflower oil.

Another embodiment comprises cyclosporin A, Norfox NP-887®, and Safflower oil.

Another embodiment comprises cyclosporin A, Norfox NP-9®, and Safflower oil.

Another embodiment comprises cyclosporin A, Norfox NP-977®, and Safflower oil.

Another embodiment comprises cyclosporin A, Norfox OP-100®, and Safflower oil.

Another embodiment comprises cyclosporin A, Norfox OP-102®, and Safflower oil.

Another embodiment comprises cyclosporin A, Norfox OP-114®, and Safflower oil.

Another embodiment comprises cyclosporin A, Norfox OP-45®, and Safflower oil.

Another embodiment comprises cyclosporin A, Pegosperse 100-L®, and Safflower oil.

Another embodiment comprises cyclosporin A, Pegosperse 100-O®, and Safflower oil.

Another embodiment comprises cyclosporin A, Pegosperse 100-S®, and Safflower oil.

Another embodiment comprises cyclosporin A, Pegosperse 1500-MS®, and Safflower oil.

Another embodiment comprises cyclosporin A, Pegosperse 1750-MS®, and Safflower oil.

Another embodiment comprises cyclosporin A, Pegosperse 200 DL®, and Safflower oil.

Another embodiment comprises cyclosporin A, Pegosperse 200-ML®, and Safflower oil.

Another embodiment comprises cyclosporin A, Pegosperse 400-DL®, and Safflower oil.

Another embodiment comprises cyclosporin A, Pegosperse 400-DO®, and Safflower oil.

Another embodiment comprises cyclosporin A, Pegosperse 400-DS®, and Safflower oil.

Another embodiment comprises cyclosporin A, Pegosperse 400-ML®, and Safflower oil.

Another embodiment comprises cyclosporin A, Pegosperse 400-MO®, and Safflower oil.

Another embodiment comprises cyclosporin A, Pegosperse 400-MOT®, and Safflower oil.

Another embodiment comprises cyclosporin A, Pegosperse 400-MS®, and Safflower oil.

Another embodiment comprises cyclosporin A, Pegosperse 50-DS®, and Safflower oil.

Another embodiment comprises cyclosporin A, Pegosperse 50-MS®, and Safflower oil.

Another embodiment comprises cyclosporin A, Pegosperse 600-ML®, and Safflower oil.

Another embodiment comprises cyclosporin A, Pegosperse 600-MS®, and Safflower oil.

Another embodiment comprises cyclosporin A, Plurafac A-38®, and Safflower oil.

Another embodiment comprises cyclosporin A, Plurafac B-25-5®, and Safflower oil.

Another embodiment comprises cyclosporin A, Plurafac B-26®, and Safflower oil.

Another embodiment comprises cyclosporin A, Plurafac D-25®, and Safflower oil.

Another embodiment comprises cyclosporin A, Plurafac LF 131®, and Safflower oil.

Another embodiment comprises cyclosporin A, Plurafac LF 4030®, and Safflower oil.

Another embodiment comprises cyclosporin A, Plurafac LF 7000®, and Safflower oil.

Another embodiment comprises cyclosporin A, Plurafac RA-20®, and Safflower oil.

Another embodiment comprises cyclosporin A, Plurafac RA-30®, and Safflower oil.

Another embodiment comprises cyclosporin A, Plurafac RA-40®, and Safflower oil.

Another embodiment comprises cyclosporin A, Pluronic 10R5®, and Safflower oil.

Another embodiment comprises cyclosporin A, Pluronic 17R2®, and Safflower oil.

Another embodiment comprises cyclosporin A, Pluronic 17R4®, and Safflower oil.

Another embodiment comprises cyclosporin A, Pluronic 25R2®, and Safflower oil.

Another embodiment comprises cyclosporin A, Pluronic 25R4®, and Safflower oil.

Another embodiment comprises cyclosporin A, Pluronic 31R1®, and Safflower oil.

Another embodiment comprises cyclosporin A, Pluronic F108®, and Safflower oil.

Another embodiment comprises cyclosporin A, Pluronic F127®, and Safflower oil.

Another embodiment comprises cyclosporin A, Pluronic F38®, and Safflower oil.

Another embodiment comprises cyclosporin A, Pluronic F68®, and Safflower oil.

Another embodiment comprises cyclosporin A, Pluronic F68LF®, and Safflower oil.

Another embodiment comprises cyclosporin A, Pluronic F77®, and Safflower oil.

Another embodiment comprises cyclosporin A, Pluronic F87®, and Safflower oil.

Another embodiment comprises cyclosporin A, Pluronic F88®, and Safflower oil.

Another embodiment comprises cyclosporin A, Pluronic F98®, and Safflower oil.

Another embodiment comprises cyclosporin A, Pluronic L10®, and Safflower oil.

Another embodiment comprises cyclosporin A, Pluronic L101®, and Safflower oil.

Another embodiment comprises cyclosporin A, Pluronic L121®, and Safflower oil.

Another embodiment comprises cyclosporin A, Pluronic L31®, and Safflower oil.

Another embodiment comprises cyclosporin A, Pluronic L35®, and Safflower oil.

Another embodiment comprises cyclosporin A, Pluronic L43®, and Safflower oil.

Another embodiment comprises cyclosporin A, Pluronic L44®, and Safflower oil.

Another embodiment comprises cyclosporin A, Pluronic L61®, and Safflower oil.

Another embodiment comprises cyclosporin A, Pluronic L62®, and Safflower oil.

Another embodiment comprises cyclosporin A, Pluronic L62D®, and Safflower oil.

Another embodiment comprises cyclosporin A, Pluronic L62LF®, and Safflower oil.

Another embodiment comprises cyclosporin A, Pluronic L64®, and Safflower oil.

Another embodiment comprises cyclosporin A, Pluronic L81®, and Safflower oil.

Another embodiment comprises cyclosporin A, Pluronic L92®, and Safflower oil.

Another embodiment comprises cyclosporin A, Pluronic P103®, and Safflower oil.

Another embodiment comprises cyclosporin A, Pluronic P104®, and Safflower oil.

Another embodiment comprises cyclosporin A, Pluronic P105®, and Safflower oil.

Another embodiment comprises cyclosporin A, Pluronic P123®, and Safflower oil.

Another embodiment comprises cyclosporin A, Pluronic P65®, and Safflower oil.

Another embodiment comprises cyclosporin A, Pluronic P84®, and Safflower oil.

Another embodiment comprises cyclosporin A, Pluronic P85®, and Safflower oil.

Another embodiment comprises cyclosporin A, Polyaldo 10-2-P®, and Safflower oil.

Another embodiment comprises cyclosporin A, Polyaldo DGDO®, and Safflower oil.

Another embodiment comprises cyclosporin A, Polyaldo HGDS®, and Safflower oil.

Another embodiment comprises cyclosporin A, Polyaldo TGMS®, and Safflower oil.

Another embodiment comprises cyclosporin A, Protegin V®, and Safflower oil.

Another embodiment comprises cyclosporin A, Protegin W®, and Safflower oil.

Another embodiment comprises cyclosporin A, Protegin WX®, and Safflower oil.

Another embodiment comprises cyclosporin A, Protegin XV®, and Safflower oil.

Another embodiment comprises cyclosporin A, Rhodasurf BC-420®, and Safflower oil.

Another embodiment comprises cyclosporin A, Rhodasurf BC-610®, and Safflower oil.

Another embodiment comprises cyclosporin A, Rhodasurf BC-630®, and Safflower oil.

Another embodiment comprises cyclosporin A, Rhodasurf BC-720®, and Safflower oil.

Another embodiment comprises cyclosporin A, Rhodasurf BC-840®, and Safflower oil.

Another embodiment comprises cyclosporin A, Rhodasurf L 1®, and Safflower oil.

Another embodiment comprises cyclosporin A, Rhodasurf L 12®, and Safflower oil.

Another embodiment comprises cyclosporin A, Rhodasurf L 2®, and Safflower oil.

Another embodiment comprises cyclosporin A, Rhodasurf L 20®, and Safflower oil.

Another embodiment comprises cyclosporin A, Rhodasurf L 3®, and Safflower oil.

Another embodiment comprises cyclosporin A, Rhodasurf L 30®, and Safflower oil.

Another embodiment comprises cyclosporin A, Rhodasurf L 4®, and Safflower oil.

Another embodiment comprises cyclosporin A, Rhodasurf L 50®, and Safflower oil.

Another embodiment comprises cyclosporin A, Rhodasurf L 7/90®, and Safflower oil.

Another embodiment comprises cyclosporin A, Rhodasurf LA-3®, and Safflower oil.

Another embodiment comprises cyclosporin A, Rhodasurf LA-7®, and Safflower oil.

Another embodiment comprises cyclosporin A, Rhodasurf LAN-23®, and Safflower oil.

Another embodiment comprises cyclosporin A, Rhodasurf ON-870®, and Safflower oil.

Another embodiment comprises cyclosporin A, Rhodasurf ON-877®, and Safflower oil.

Another embodiment comprises cyclosporin A, Rhodasurf TDA-8/5®, and Safflower oil.

Another embodiment comprises cyclosporin A, Ritapro 100®, and Safflower oil.

Another embodiment comprises cyclosporin A, Ritapro 165®, and Safflower oil.

Another embodiment comprises cyclosporin A, Ritapro 200®, and Safflower oil.

Another embodiment comprises cyclosporin A, Ritapro 300®, and Safflower oil.

Another embodiment comprises cyclosporin A, Ritoleth 10®, and Safflower oil.

Another embodiment comprises cyclosporin A, Ritoleth 2®, and Safflower oil.

Another embodiment comprises cyclosporin A, Ritoleth 20®, and Safflower oil.

Another embodiment comprises cyclosporin A, Ritoleth 5®, and Safflower oil.

Another embodiment comprises cyclosporin A, Santone 10-10-0®, and Safflower oil.

Another embodiment comprises cyclosporin A, Santone 3-1-S XTR®, and Safflower oil.

Another embodiment comprises cyclosporin A, Santone 3-1-SH®, and Safflower oil.

Another embodiment comprises cyclosporin A, Santone 8-1-O®, and Safflower oil.

Another embodiment comprises cyclosporin A, Solulan 16, and Safflower oil.

Another embodiment comprises cyclosporin A, Solulan 75®, and Safflower oil.

Another embodiment comprises cyclosporin A, Solulan C-24®, and Safflower oil.

Another embodiment comprises cyclosporin A, Solulan L-575®, and Safflower oil.

Another embodiment comprises cyclosporin A, Standamox CAW®, and Safflower oil.

Another embodiment comprises cyclosporin A, Surfine AZI-A®, and Safflower oil.

Another embodiment comprises cyclosporin A, Surfonic CO-15®, and Safflower oil.

Another embodiment comprises cyclosporin A, Surfonic CO-25®, and Safflower oil.

Another embodiment comprises cyclosporin A, Surfonic CO-30®, and Safflower oil.

Another embodiment comprises cyclosporin A, Surfonic CO-36®, and Safflower oil.

Another embodiment comprises cyclosporin A, Surfonic CO-42®, and Safflower oil.

Another embodiment comprises cyclosporin A, Surfonic E400-MO®, and Safflower oil.

Another embodiment comprises cyclosporin A, Surfonic L12-3®, and Safflower oil.

Another embodiment comprises cyclosporin A, Surfonic L12-6®, and Safflower oil.

Another embodiment comprises cyclosporin A, Surfonic L12-8®, and Safflower oil.

Another embodiment comprises cyclosporin A, Surfonic L24-12®, and Safflower oil.

Another embodiment comprises cyclosporin A, Surfonic L24-2®, and Safflower oil.

Another embodiment comprises cyclosporin A, Surfonic L24-22®, and Safflower oil.

Another embodiment comprises cyclosporin A, Surfonic L24-3®, and Safflower oil.

Another embodiment comprises cyclosporin A, Surfonic L24-4®, and Safflower oil.

Another embodiment comprises cyclosporin A, Surfonic L24-5®, and Safflower oil.

Another embodiment comprises cyclosporin A, Surfonic L24-7®, and Safflower oil.

Another embodiment comprises cyclosporin A, Surfonic L24-7.1®, and Safflower oil.

Another embodiment comprises cyclosporin A, Surfonic L24-9®, and Safflower oil.

Another embodiment comprises cyclosporin A, Surfonic LF-17®, and Safflower oil.

Another embodiment comprises cyclosporin A, Surfonic LF-18®, and Safflower oil.

Another embodiment comprises cyclosporin A, Surfonic LF-37®, and Safflower oil.

Another embodiment comprises cyclosporin A, Surfonic LF-41®, and Safflower oil.

Another embodiment comprises cyclosporin A, Surfonic LF-42®, and Safflower oil.

Another embodiment comprises cyclosporin A, Surfonic LF-47®, and Safflower oil.

Another embodiment comprises cyclosporin A, Surfonic LF-68®, and Safflower oil..

Another embodiment comprises cyclosporin A, Surfonic N-10®, and Safflower oil.

Another embodiment comprises cyclosporin A, Surfonic N-100®, and Safflower oil.

Another embodiment comprises cyclosporin A, Surfonic N-102®, and Safflower oil.

Another embodiment comprises cyclosporin A, Surfonic N-120®, and Safflower oil.

Another embodiment comprises cyclosporin A, Surfonic N-150®, and Safflower oil.

Another embodiment comprises cyclosporin A, Surfonic N-200®, and Safflower oil.

Another embodiment comprises cyclosporin A, Surfonic N-300®, and Safflower oil.

Another embodiment comprises cyclosporin A, Surfonic N-31.5®, and Safflower oil.

Another embodiment comprises cyclosporin A, Surfonic N-40®, and Safflower oil.

Another embodiment comprises cyclosporin A, Surfonic N-400®, and Safflower oil.

Another embodiment comprises cyclosporin A, Surfonic N-60®, and Safflower oil.

Another embodiment comprises cyclosporin A, Surfonic N-85®, and Safflower oil.

Another embodiment comprises cyclosporin A, Surfonic N-95®, and Safflower oil.

Another embodiment comprises cyclosporin A, Surfonic NB-18®, and Safflower oil.

Another embodiment comprises cyclosporin A, Surfonic NB-23®, and Safflower oil.

Another embodiment comprises cyclosporin A, Surfonic NB-25®, and Safflower oil.

Another embodiment comprises cyclosporin A, Surfonic NB-307®, and Safflower oil.

Another embodiment comprises cyclosporin A, Surfonic NB-31®, and Safflower oil.

Another embodiment comprises cyclosporin A, Surfonic NB-41®, and Safflower oil.

Another embodiment comprises cyclosporin A, Surfonic NB-43®, and Safflower oil.

Another embodiment comprises cyclosporin A, Surfonic OP-100®, and Safflower oil.

Another embodiment comprises cyclosporin A, Surfonic OP-120®, and Safflower oil.

Another embodiment comprises cyclosporin A, Surfonic OP-15®, and Safflower oil.

Another embodiment comprises cyclosporin A, Surfonic OP-35®, and Safflower oil.

Another embodiment comprises cyclosporin A, Surfonic OP-70®, and Safflower oil.

Another embodiment comprises cyclosporin A, Surfonic P1®, and Safflower oil.

Another embodiment comprises cyclosporin A, Surfonic P3®, and Safflower oil.

Another embodiment comprises cyclosporin A, Surfonic P5®, and Safflower oil.

Another embodiment comprises cyclosporin A, Surfonic P6®, and Safflower oil.

Another embodiment comprises cyclosporin A, Surfonic POA-17R2®, and Safflower oil.

Another embodiment comprises cyclosporin A, Surfonic POA-25R2®, and Safflower oil.

Another embodiment comprises cyclosporin A, Surfonic TDA-11®, and Safflower oil.

Another embodiment comprises cyclosporin A, Surfonic TDA-3B®, and Safflower oil.

Another embodiment comprises cyclosporin A, Surfonic TDA-6®, and Safflower oil.

Another embodiment comprises cyclosporin A, Surfonic TDA-6/88®, and Safflower oil.

Another embodiment comprises cyclosporin A, Surfonic TDA-8®, and Safflower oil.

Another embodiment comprises cyclosporin A, Surfonic TDA-8/90®, and Safflower oil.

Another embodiment comprises cyclosporin A, Surfonic TDA-9®, and Safflower oil.

Another embodiment comprises cyclosporin A, Surfonic TX-CCN®, and Safflower oil.

Another embodiment comprises cyclosporin A, Tagat CH 40®, and Safflower oil.

Another embodiment comprises cyclosporin A, Tagat CH 60®, and Safflower oil.

Another embodiment comprises cyclosporin A, Tagat L 2®, and Safflower oil.

Another embodiment comprises cyclosporin A, Tagat S®, and Safflower oil.

Another embodiment comprises cyclosporin A, Tagat S 2®, and Safflower oil.

Another embodiment comprises cyclosporin A, Tally 100 Plus®, and Safflower oil.

Another embodiment comprises cyclosporin A, T-Det A106®, and Safflower oil.

Another embodiment comprises cyclosporin A, T-Det A109®, and Safflower oil.

Another embodiment comprises cyclosporin A, T-Det A136®, and Safflower oil.

Another embodiment comprises cyclosporin A, T-Det A139®, and Safflower oil.

Another embodiment comprises cyclosporin A, T-Det A2412®, and Safflower oil.

Another embodiment comprises cyclosporin A, T-Det A243®, and Safflower oil.

Another embodiment comprises cyclosporin A, T-Det A247®, and Safflower oil.

Another embodiment comprises cyclosporin A, T-Det A249®, and Safflower oil.

Another embodiment comprises cyclosporin A, T-Det A467®, and Safflower oil.

Another embodiment comprises cyclosporin A, T-Det COE®, and Safflower oil.

Another embodiment comprises cyclosporin A, T-Det DD-10®, and Safflower oil.

Another embodiment comprises cyclosporin A, T-Det DD-5®, and Safflower oil.

Another embodiment comprises cyclosporin A, T-Det DD-7®, and Safflower oil.

Another embodiment comprises cyclosporin A, T-Det EPO-104®, and Safflower oil.

Another embodiment comprises cyclosporin A, T-Det EPO-61®, and Safflower oil.

Another embodiment comprises cyclosporin A, T-Det EPO-62®, and Safflower oil.

Another embodiment comprises cyclosporin A, T-Det EPO-64®, and Safflower oil.

Another embodiment comprises cyclosporin A, T-Det N-1.5®, and Safflower oil.

Another embodiment comprises cyclosporin A, T-Det N-10.5®, and Safflower oil.

Another embodiment comprises cyclosporin A, T-Det N-100®, and Safflower oil.

Another embodiment comprises cyclosporin A, T-Det N-1007®, and Safflower oil.

Another embodiment comprises cyclosporin A, T-Det N-12®, and Safflower oil.

Another embodiment comprises cyclosporin A, T-Det N-14®, and Safflower oil.

Another embodiment comprises cyclosporin A, T-Det N-20®, and Safflower oil.

Another embodiment comprises cyclosporin A, T-Det N-30®, and Safflower oil.

Another embodiment comprises cyclosporin A, T-Det N-307®, and Safflower oil.

Another embodiment comprises cyclosporin A, T-Det N-4®, and Safflower oil.

Another embodiment comprises cyclosporin A, T-Det N-40®, and Safflower oil.

Another embodiment comprises cyclosporin A, T-Det N-407®, and Safflower oil.

Another embodiment comprises cyclosporin A, T-Det N-50®, and Safflower oil.

Another embodiment comprises cyclosporin A, T-Det N-507®, and Safflower oil.

Another embodiment comprises cyclosporin A, T-Det N-6®, and Safflower oil.

Another embodiment comprises cyclosporin A, T-Det N-70®, and Safflower oil.

Another embodiment comprises cyclosporin A, T-Det N-8®, and Safflower oil.

Another embodiment comprises cyclosporin A, T-Det N-9.5®, and Safflower oil.

Another embodiment comprises cyclosporin A, T-Det O-12®, and Safflower oil.

Another embodiment comprises cyclosporin A, T-Det O-165®, and Safflower oil.

Another embodiment comprises cyclosporin A, T-Det O-307®, and Safflower oil.

Another embodiment comprises cyclosporin A, T-Det O-40®, and Safflower oil.

Another embodiment comprises cyclosporin A, T-Det O-407®, and Safflower oil.

Another embodiment comprises cyclosporin A, T-Det O-6®, and Safflower oil.

Another embodiment comprises cyclosporin A, T-Det O-8®, and Safflower oil.

Another embodiment comprises cyclosporin A, T-Det O-9®, and Safflower oil.

Another embodiment comprises cyclosporin A, Tegin®, and Safflower oil.

Another embodiment comprises cyclosporin A, Tergitol 15-S-12®, and Safflower oil.

Another embodiment comprises cyclosporin A, Tergitol 15-S-15®, and Safflower oil.

Another embodiment comprises cyclosporin A, Tergitol 15-S-20®, and Safflower oil.

Another embodiment comprises cyclosporin A, Tergitol 15-S-20 (80AQ)®, and Safflower oil.

Another embodiment comprises cyclosporin A, Tergitol 15-S-3®, and Safflower oil.

Another embodiment comprises cyclosporin A, Tergitol 15-S-30®, and Safflower oil.

Another embodiment comprises cyclosporin A, Tergitol 15-S-40®, and Safflower oil.

Another embodiment comprises cyclosporin A, Tergitol 15-S-40 (70AQ)®, and Safflower oil.

Another embodiment comprises cyclosporin A, Tergitol 15-S-5®, and Safflower oil.

Another embodiment comprises cyclosporin A, Tergitol 15-S-7®, and Safflower oil.

Another embodiment comprises cyclosporin A, Tergitol 15-S-9®, and Safflower oil.

Another embodiment comprises cyclosporin A, Tergitol L-101®, and Safflower oil.

Another embodiment comprises cyclosporin A, Tergitol L-61®, and Safflower oil.

Another embodiment comprises cyclosporin A, Tergitol L-62®, and Safflower oil.

Another embodiment comprises cyclosporin A, Tergitol L-64®, and Safflower oil.

Another embodiment comprises cyclosporin A, Tergitol L-81®, and Safflower oil.

Another embodiment comprises cyclosporin A, Tergitol NP-10®, and Safflower oil.

Another embodiment comprises cyclosporin A, Tergitol NP-11®, and Safflower oil.

Another embodiment comprises cyclosporin A, Tergitol NP-12®, and Safflower oil.

Another embodiment comprises cyclosporin A, Tergitol NP-13®, and Safflower oil.

Another embodiment comprises cyclosporin A, Tergitol NP-15®, and Safflower oil.

Another embodiment comprises cyclosporin A, Tergitol NP-30®, and Safflower oil.

Another embodiment comprises cyclosporin A, Tergitol NP-30 (70 AQ)®, and Safflower oil.

Another embodiment comprises cyclosporin A, Tergitol NP-4®, and Safflower oil.

Another embodiment comprises cyclosporin A, Tergitol NP-40®, and Safflower oil.

Another embodiment comprises cyclosporin A, Tergitol NP-40 (70 AQ)®, and Safflower oil.

Another embodiment comprises cyclosporin A, Tergitol NP-50 (70 AQ)®, and Safflower oil.

Another embodiment comprises cyclosporin A, Tergitol NP-55 (70 AQ)®, and Safflower oil.

Another embodiment comprises cyclosporin A, Tergitol NP-6®, and Safflower oil.

Another embodiment comprises cyclosporin A, Tergitol NP-7®, and Safflower oil.

Another embodiment comprises cyclosporin A, Tergitol NP-70 (70 AQ)®, and Safflower oil.

Another embodiment comprises cyclosporin A, Tergitol NP-8®, and Safflower oil.

Another embodiment comprises cyclosporin A, Tergitol NP-9®, and Safflower oil.

Another embodiment comprises cyclosporin A, Tergitol NP-9.5®, and Safflower oil.

Another embodiment comprises cyclosporin A, Tergitol TMN-10®, and Safflower oil.

Another embodiment comprises cyclosporin A, Tergitol TMN-3®, and Safflower oil.

Another embodiment comprises cyclosporin A, Tergitol TMN-6®, and Safflower oil.

Another embodiment comprises cyclosporin A, Tergitol XD®, and Safflower oil.

Another embodiment comprises cyclosporin A, Tergitol XH®, and Safflower oil.

Another embodiment comprises cyclosporin A, Tergitol XJ®, and Safflower oil.

Another embodiment comprises cyclosporin A, Tetronic 1107®, and Safflower oil.

Another embodiment comprises cyclosporin A, Tetronic 1307®, and Safflower oil.

Another embodiment comprises cyclosporin A, Tetronic 150R1®, and Safflower oil.

Another embodiment comprises cyclosporin A, Tetronic 304®, and Safflower oil.

Another embodiment comprises cyclosporin A, Tetronic 701®, and Safflower oil.

Another embodiment comprises cyclosporin A, Tetronic 901®, and Safflower oil.

Another embodiment comprises cyclosporin A, Tetronic 904®, and Safflower oil.

Another embodiment comprises cyclosporin A, Tetronic 908®, and Safflower oil.

Another embodiment comprises cyclosporin A, Tetronic 90R4®, and Safflower oil.

Another embodiment comprises cyclosporin A, Tomadol 1-3®, and Safflower oil.

Another embodiment comprises cyclosporin A, Tomadol 1-5®, and Safflower oil.

Another embodiment comprises cyclosporin A, Tomadol 1-7®, and Safflower oil.

Another embodiment comprises cyclosporin A, Tomadol 23-1®, and Safflower oil.

Another embodiment comprises cyclosporin A, Tomadol 23-3®, and Safflower oil.

Another embodiment comprises cyclosporin A, Tomadol 23-5®, and Safflower oil.

Another embodiment comprises cyclosporin A, Tomadol 23-6.5®, and Safflower oil.

Another embodiment comprises cyclosporin A, Tomadol 25-12®, and Safflower oil.

Another embodiment comprises cyclosporin A, Tomadol 25-3®, and Safflower oil.

Another embodiment comprises cyclosporin A, Tomadol 25-7®, and Safflower oil.

Another embodiment comprises cyclosporin A, Tomadol 25-9®, and Safflower oil.

Another embodiment comprises cyclosporin A, Tomadol 45-13®, and Safflower oil.

Another embodiment comprises cyclosporin A, Tomadol 45-2.5®, and Safflower oil.

Another embodiment comprises cyclosporin A, Tomadol 45-7®, and Safflower oil.

Another embodiment comprises cyclosporin A, Tomadol 91-2.5®, and Safflower oil.

Another embodiment comprises cyclosporin A, Tomadol 91-6®, and Safflower oil.

Another embodiment comprises cyclosporin A, Tomadol 91-8®, and Safflower oil.

Another embodiment comprises cyclosporin A, Triton X-100®, and Safflower oil.

Another embodiment comprises cyclosporin A, Triton X-102®, and Safflower oil.

Another embodiment comprises cyclosporin A, Triton X-114®, and Safflower oil.

Another embodiment comprises cyclosporin A, Triton X-15®, and Safflower oil.

Another embodiment comprises cyclosporin A, Triton X-165 (70%)®, and Safflower oil.

Another embodiment comprises cyclosporin A, Triton X-207®, and Safflower oil.

Another embodiment comprises cyclosporin A, Triton X-305 (70%)®, and Safflower oil.

Another embodiment comprises cyclosporin A, Triton X-35®, and Safflower oil.

Another embodiment comprises cyclosporin A, Triton X-405 (70%)®, and Safflower oil.

Another embodiment comprises cyclosporin A, Triton X-45®, and Safflower oil.

Another embodiment comprises cyclosporin A, Triton X-705 (70%)®, and Safflower oil.

Another embodiment comprises cyclosporin A, Volpo S-10®, and Safflower oil.

Another embodiment comprises cyclosporin A, Volpo S-2®, and Safflower oil.

Another embodiment comprises cyclosporin A, Volpo S-20®, and Safflower oil.

Another embodiment comprises cyclosporin A, Volpo-10®, and Safflower oil.

Another embodiment comprises cyclosporin A, Volpo-20®, and Safflower oil.

Another embodiment comprises cyclosporin A, Volpo-3®, and Safflower oil.

Another embodiment comprises cyclosporin A, Volpo-5®, and Safflower oil.

Another embodiment comprises cyclosporin A, Abex 2515®, and Maize oil.

Another embodiment comprises cyclosporin A, Abex 2525/40®, and Maize oil.

Another embodiment comprises cyclosporin A, Abex 2535®, and Maize oil.

Another embodiment comprises cyclosporin A, Abex 2545®, and Maize oil.

Another embodiment comprises cyclosporin A, Acconon E®, and Maize oil.

Another embodiment comprises cyclosporin A, Alfonic 1216CO-9®, and Maize oil.

Another embodiment comprises cyclosporin A, Alfonic 1412-3®, and Maize oil.

Another embodiment comprises cyclosporin A, Alfonic TDA-12®, and Maize oil.

Another embodiment comprises cyclosporin A, Alfonic TDA-3®, and Maize oil.

Another embodiment comprises cyclosporin A, Alfonic TDA-4®, and Maize oil.

Another embodiment comprises cyclosporin A, Alfonic TDA-6®, and Maize oil.

Another embodiment comprises cyclosporin A, Alfonic TDA-7®, and Maize oil.

Another embodiment comprises cyclosporin A, Alfonic TDA-8®, and Maize oil.

Another embodiment comprises cyclosporin A, Alfonic TDA-9®, and Maize oil.

Another embodiment comprises cyclosporin A, Alkamuls 400-DO®, and Maize oil.

Another embodiment comprises cyclosporin A, Alkamuls 400-MO/E®, and Maize oil.

Another embodiment comprises cyclosporin A, Ameroxol OE-10®, and Maize oil.

Another embodiment comprises cyclosporin A, Ameroxol OE-2®, and Maize oil.

Another embodiment comprises cyclosporin A, Ameroxol OE-20®, and Maize oil.

Another embodiment comprises cyclosporin A, Armix 176®, and Maize oil.

Another embodiment comprises cyclosporin A, Armix 180-C®, and Maize oil.

Another embodiment comprises cyclosporin A, Armix 183®, and Maize oil.

Another embodiment comprises cyclosporin A, Brij 35®, and Maize oil.

Another embodiment comprises cyclosporin A, Brij 35 Liquid®, and Maize oil.

Another embodiment comprises cyclosporin A, Brij 35 SP®, and Maize oil.

Another embodiment comprises cyclosporin A, Canasol MJ 2109®, and Maize oil.

Another embodiment comprises cyclosporin A, Canasol MJ 52®, and Maize oil.

Another embodiment comprises cyclosporin A, Canasol R 3600®, and Maize oil.

Another embodiment comprises cyclosporin A, Canasol R 3603®, and Maize oil.

Another embodiment comprises cyclosporin A, Canasol R 4000 H®, and Maize oil.

Another embodiment comprises cyclosporin A, Cedepal CA-210®, and Maize oil.

Another embodiment comprises cyclosporin A, Cedepal CA-520®, and Maize oil.

Another embodiment comprises cyclosporin A, Cedepal CA-630®, and Maize oil.

Another embodiment comprises cyclosporin A, Cedepal CA-720®, and Maize oil.

Another embodiment comprises cyclosporin A, Cedepal CA-890®, and Maize oil.

Another embodiment comprises cyclosporin A, Cedepal CA-897®, and Maize oil.

Another embodiment comprises cyclosporin A, Cedepal CO-210®, and Maize oil.

Another embodiment comprises cyclosporin A, Cedepal CO-430®, and Maize oil.

Another embodiment comprises cyclosporin A, Cedepal CO-530®, and Maize oil.

Another embodiment comprises cyclosporin A, Cedepal CO-630®, and Maize oil.

Another embodiment comprises cyclosporin A, Cedepal CO-710®, and Maize oil.

Another embodiment comprises cyclosporin A, Cedepal CO-730®, and Maize oil.

Another embodiment comprises cyclosporin A, Cedepal CO-880®, and Maize oil.

Another embodiment comprises cyclosporin A, Cedepal CO-887®, and Maize oil.

Another embodiment comprises cyclosporin A, Cedepal CO-890®, and Maize oil.

Another embodiment comprises cyclosporin A, Cedepal CO-897®, and Maize oil.

Another embodiment comprises cyclosporin A, Cedepal CO-970®, and Maize oil.

Another embodiment comprises cyclosporin A, Cedepal CO-977®, and Maize oil.

Another embodiment comprises cyclosporin A, Cedepal CO-990®, and Maize oil.

Another embodiment comprises cyclosporin A, Cedepal CO-997®, and Maize oil.

Another embodiment comprises cyclosporin A, Chemal BP 261®, and Maize oil.

Another embodiment comprises cyclosporin A, Chemal BP 262®, and Maize oil.

Another embodiment comprises cyclosporin A, Chemal BP 3172®, and Maize oil.

Another embodiment comprises cyclosporin A, Chemal BP 3174®, and Maize oil.

Another embodiment comprises cyclosporin A, Chemal DA-4®, and Maize oil.

Another embodiment comprises cyclosporin A, Chemal DA-6®, and Maize oil.

Another embodiment comprises cyclosporin A, Chemal DA-9®, and Maize oil.

Another embodiment comprises cyclosporin A, Chemal LA-23®, and Maize oil.

Another embodiment comprises cyclosporin A, Chemal LA-4®, and Maize oil.

Another embodiment comprises cyclosporin A, Chemal LA-9®, and Maize oil.

Another embodiment comprises cyclosporin A, Chemal OA-20G®, and Maize oil.

Another embodiment comprises cyclosporin A, Chemal OA-5®, and Maize oil.

Another embodiment comprises cyclosporin A, Chemal OA-9®, and Maize oil.

Another embodiment comprises cyclosporin A, Chemal TDA-12®, and Maize oil.

Another embodiment comprises cyclosporin A, Chemal TDA-15®, and Maize oil.

Another embodiment comprises cyclosporin A, Chemal TDA-3®, and Maize oil.

Another embodiment comprises cyclosporin A, Chemal TDA-6®, and Maize oil.

Another embodiment comprises cyclosporin A, Chemal TDA-9®, and Maize oil.

Another embodiment comprises cyclosporin A, Chemax CO-16®, and Maize oil.

Another embodiment comprises cyclosporin A, Chemax CO-200/50®, and Maize oil.

Another embodiment comprises cyclosporin A, Chemax CO-25®, and Maize oil.

Another embodiment comprises cyclosporin A, Chemax CO-30®, and Maize oil.

Another embodiment comprises cyclosporin A, Chemax CO-36®, and Maize oil.

Another embodiment comprises cyclosporin A, Chemax CO-40®, and Maize oil.

Another embodiment comprises cyclosporin A, Chemax CO-5®, and Maize oil.

Another embodiment comprises cyclosporin A, Chemax CO-80®, and Maize oil.

Another embodiment comprises cyclosporin A, Chemax DNP-150/50®, and Maize oil.

Another embodiment comprises cyclosporin A, Chemax DNP-18®, and Maize oil.

Another embodiment comprises cyclosporin A, Chemax DNP-8®, and Maize oil.

Another embodiment comprises cyclosporin A, Chemax E 200 ML®, and Maize oil.

Another embodiment comprises cyclosporin A, Chemax E 200 MO®, and Maize oil.

Another embodiment comprises cyclosporin A, Chemax E 400 ML®, and Maize oil.

Another embodiment comprises cyclosporin A, Chemax E 400 MO®, and Maize oil.

Another embodiment comprises cyclosporin A, Chemax E 600 ML®, and Maize oil.

Another embodiment comprises cyclosporin A, Chemax E 600 MO®, and Maize oil.

Another embodiment comprises cyclosporin A, Chemax E1000 MO®, and Maize oil.

Another embodiment comprises cyclosporin A, Chemax E1000 MS®, and Maize oil.

Another embodiment comprises cyclosporin A, Chemax E200 MS®, and Maize oil.

Another embodiment comprises cyclosporin A, Chemax E400 MS®, and Maize oil.

Another embodiment comprises cyclosporin A, Chemax E600 MS®, and Maize oil.

Another embodiment comprises cyclosporin A, Chemax HCO-16®, and Maize oil.

Another embodiment comprises cyclosporin A, Chemax HCO-200/50®, and Maize oil.

Another embodiment comprises cyclosporin A, Chemax HCO-25®, and Maize oil.

Another embodiment comprises cyclosporin A, Chemax HCO-5®, and Maize oil.

Another embodiment comprises cyclosporin A, Chemax TO-16®, and Maize oil.

Another embodiment comprises cyclosporin A, Chemax TO-8®, and Maize oil.

Another embodiment comprises cyclosporin A, Colalube 3409®, and Maize oil.

Another embodiment comprises cyclosporin A, Colalube 3411®, and Maize oil.

Another embodiment comprises cyclosporin A, Colalube 3413®, and Maize oil.

Another embodiment comprises cyclosporin A, Colalube 3414®, and Maize oil.

Another embodiment comprises cyclosporin A, Colalube 3415®, and Maize oil.

Another embodiment comprises cyclosporin A, Colalube 3416®, and Maize oil.

Another embodiment comprises cyclosporin A, Colalube 3417®, and Maize oil.

Another embodiment comprises cyclosporin A, Delonic NPE-100®, and Maize oil.

Another embodiment comprises cyclosporin A, Delonic NPE-30®, and Maize oil.

Another embodiment comprises cyclosporin A, Delonic NPE-40®, and Maize oil.

Another embodiment comprises cyclosporin A, Delonic OPE-10®, and Maize oil.

Another embodiment comprises cyclosporin A, Delonic OPE-12®, and Maize oil.

Another embodiment comprises cyclosporin A, Delonic OPE-30®, and Maize oil.

Another embodiment comprises cyclosporin A, Delonic OPE-40®, and Maize oil.

Another embodiment comprises cyclosporin A, Delonic OPE-5®, and Maize oil.

Another embodiment comprises cyclosporin A, Delonic OPE-7.5®, and Maize oil.

Another embodiment comprises cyclosporin A, DePeg 16-CO®, and Maize oil.

Another embodiment comprises cyclosporin A, DePeg 25-CO®, and Maize oil.

Another embodiment comprises cyclosporin A, DePeg 30-CO®, and Maize oil.

Another embodiment comprises cyclosporin A, DePeg 40-CO®, and Maize oil.

Another embodiment comprises cyclosporin A, DePeg 5-CO®, and Maize oil.

Another embodiment comprises cyclosporin A, DePeg 80-CO®, and Maize oil.

Another embodiment comprises cyclosporin A, DeSonic S-100®, and Maize oil.

Another embodiment comprises cyclosporin A, DeSonic S-114®, and Maize oil.

Another embodiment comprises cyclosporin A, DeSonic S-405®, and Maize oil.

Another embodiment comprises cyclosporin A, DeSonic S-45®, and Maize oil.

Another embodiment comprises cyclosporin A, DeSonic TDA-9®, and Maize oil.

Another embodiment comprises cyclosporin A, DeThox Acid O-14®, and Maize oil.

Another embodiment comprises cyclosporin A, DeThox Acid O-16®, and Maize oil.

Another embodiment comprises cyclosporin A, DeThox Acid O-9®, and Maize oil.

Another embodiment comprises cyclosporin A, DeThox Acid TO-14®, and Maize oil.

Another embodiment comprises cyclosporin A, DeThox Acid TO-16.5®, and Maize oil.

Another embodiment comprises cyclosporin A, DeThox Acid TO-8.5®, and Maize oil.

Another embodiment comprises cyclosporin A, Dur-Em 114®, and Maize oil.

Another embodiment comprises cyclosporin A, Dur-Em 117®, and Maize oil.

Another embodiment comprises cyclosporin A, Dur-Em 204®, and Maize oil.

Another embodiment comprises cyclosporin A, Dur-Em 207®, and Maize oil.

Another embodiment comprises cyclosporin A, Dur-Em 207E®, and Maize oil.

Another embodiment comprises cyclosporin A, Emerest 2704®, and Maize oil.

Another embodiment comprises cyclosporin A, Emerest 2712, and Maize oil.

Another embodiment comprises cyclosporin A, Emerest 2715®, and Maize oil.

Another embodiment comprises cyclosporin A, Ethal EH-2®, and Maize oil.

Another embodiment comprises cyclosporin A, Ethal EH-5®, and Maize oil.

Another embodiment comprises cyclosporin A, Ethal OA-23®, and Maize oil.

Another embodiment comprises cyclosporin A, Ethosperse CA-20®, and Maize oil.

Another embodiment comprises cyclosporin A, Ethosperse LA-12®, and Maize oil.

Another embodiment comprises cyclosporin A, Ethosperse LA-23®, and Maize oil.

Another embodiment comprises cyclosporin A, Ethosperse LA-4®, and Maize oil.

Another embodiment comprises cyclosporin A, Ethox 3095®, and Maize oil.

Another embodiment comprises cyclosporin A, Ethox CO-16®, and Maize oil.

Another embodiment comprises cyclosporin A, Ethox CO-200®, and Maize oil.

Another embodiment comprises cyclosporin A, Ethox CO-25®, and Maize oil.

Another embodiment comprises cyclosporin A, Ethox CO-30®, and Maize oil.

Another embodiment comprises cyclosporin A, Ethox CO-36®, and Maize oil.

Another embodiment comprises cyclosporin A, Ethox CO-40®, and Maize oil.

Another embodiment comprises cyclosporin A, Ethox CO-5®, and Maize oil.

Another embodiment comprises cyclosporin A, Ethox DO-14®, and Maize oil.

Another embodiment comprises cyclosporin A, Ethox DO-9®, and Maize oil.

Another embodiment comprises cyclosporin A, Ethox MA-8®, and Maize oil.

Another embodiment comprises cyclosporin A, Ethox MI-14®, and Maize oil.

Another embodiment comprises cyclosporin A, Ethox ML-14®, and Maize oil.

Another embodiment comprises cyclosporin A, Ethox ML-5®, and Maize oil.

Another embodiment comprises cyclosporin A, Ethox MO-14®, and Maize oil.

Another embodiment comprises cyclosporin A, Ethox MO-9®, and Maize oil.

Another embodiment comprises cyclosporin A, Ethox MS-14®, and Maize oil.

Another embodiment comprises cyclosporin A, Ethox MS-23®, and Maize oil.

Another embodiment comprises cyclosporin A, Ethox MS-40®, and Maize oil.

Another embodiment comprises cyclosporin A, Ethox MS-8®, and Maize oil.

Another embodiment comprises cyclosporin A, Ethox TO-16®, and Maize oil.

Another embodiment comprises cyclosporin A, Ethox TO-8®, and Maize oil.

Another embodiment comprises cyclosporin A, Ethylan SN-120®, and Maize oil.

Another embodiment comprises cyclosporin A, Ethylan SN-70®, and Maize oil.

Another embodiment comprises cyclosporin A, Ethylan SN-90®, and Maize oil.

Another embodiment comprises cyclosporin A, Flexricin 13®, and Maize oil.

Another embodiment comprises cyclosporin A, Flexricin 15®, and Maize oil.

Another embodiment comprises cyclosporin A, Genapol 1261®, and Maize oil.

Another embodiment comprises cyclosporin A, Generol 122 E25®, and Maize oil.

Another embodiment comprises cyclosporin A, Glicepol 160®, and Maize oil.

Another embodiment comprises cyclosporin A, Glicepol 560®, and Maize oil.

Another embodiment comprises cyclosporin A, Glicepol GMS 20®, and Maize oil.

Another embodiment comprises cyclosporin A, Hetoxide BN-13®, and Maize oil.

Another embodiment comprises cyclosporin A, Hetoxide DNP-4®, and Maize oil.

Another embodiment comprises cyclosporin A, Hetoxide DNP-9.68, and Maize oil.

Another embodiment comprises cyclosporin A, Hetoxide G-26®, and Maize oil.

Another embodiment comprises cyclosporin A, Hetoxide G-7®, and Maize oil.

Another embodiment comprises cyclosporin A, Hetoxide GT-20®, and Maize oil.

Another embodiment comprises cyclosporin A, Hetoxide HC-40®, and Maize oil.

Another embodiment comprises cyclosporin A, Hetoxol C-24®, and Maize oil.

Another embodiment comprises cyclosporin A, Hetoxol CA-10®, and Maize oil.

Another embodiment comprises cyclosporin A, Hetoxol CA-2®, and Maize oil.

Another embodiment comprises cyclosporin A, Hetoxol CA-20®, and Maize oil.

Another embodiment comprises cyclosporin A, Hetoxol CAWS®, and Maize oil.

Another embodiment comprises cyclosporin A, Hetoxol CD-3®, and Maize oil.

Another embodiment comprises cyclosporin A, Hetoxol CD-4®, and Maize oil.

Another embodiment comprises cyclosporin A, Hetoxol CS-15®, and Maize oil.

Another embodiment comprises cyclosporin A, Hetoxol CS-20®, and Maize oil.

Another embodiment comprises cyclosporin A, Hetoxol CS-20D®, and Maize oil.

Another embodiment comprises cyclosporin A, Hetoxol CS-25®, and Maize oil.

Another embodiment comprises cyclosporin A, Hetoxol CS-50®, and Maize oil.

Another embodiment comprises cyclosporin A, Hetoxol CS-9®, and Maize oil.

Another embodiment comprises cyclosporin A, Hetoxol CSA-15®, and Maize oil.

Another embodiment comprises cyclosporin A, Hetoxol D®, and Maize oil.

Another embodiment comprises cyclosporin A, Hetoxol G®, and Maize oil.

Another embodiment comprises cyclosporin A, Hetoxol J®, and Maize oil.

Another embodiment comprises cyclosporin A, Hetoxol L®, and Maize oil.

Another embodiment comprises cyclosporin A, Hetoxol L-23®, and Maize oil.

Another embodiment comprises cyclosporin A, Hetoxol L-4®, and Maize oil.

Another embodiment comprises cyclosporin A, Hetoxol L-9®, and Maize oil.

Another embodiment comprises cyclosporin A, Hetoxol LS-9®, and Maize oil.

Another embodiment comprises cyclosporin A, Hetoxol M-3®, and Maize oil.

Another embodiment comprises cyclosporin A, Hetoxol OA-10 Special®, and Maize oil.

Another embodiment comprises cyclosporin A, Hetoxol OA-20 Special®, and Maize oil.

Another embodiment comprises cyclosporin A, Hetoxol OA-3 Special®, and Maize oil.

Another embodiment comprises cyclosporin A, Hetoxol OA-5 Special®, and Maize oil.

Another embodiment comprises cyclosporin A, Hetoxol OL-10®, and Maize oil.

Another embodiment comprises cyclosporin A, Hetoxol OL-2®, and Maize oil.

Another embodiment comprises cyclosporin A, Hetoxol OL-23®, and Maize oil.

Another embodiment comprises cyclosporin A, Hetoxol OL-4®, and Maize oil.

Another embodiment comprises cyclosporin A, Hetoxol OL-40®, and Maize oil.

Another embodiment comprises cyclosporin A, Hetoxol OL-5®, and Maize oil.

Another embodiment comprises cyclosporin A, Hetoxol PLA®, and Maize oil.

Another embodiment comprises cyclosporin A, Hetoxol SP-15®, and Maize oil.

Another embodiment comprises cyclosporin A, Hetoxol STA-10®, and Maize oil.

Another embodiment comprises cyclosporin A, Hetoxol STA-2®, and Maize oil.

Another embodiment comprises cyclosporin A, Hetoxol STA-30®, and Maize oil.

Another embodiment comprises cyclosporin A, Hetoxol TD-12®, and Maize oil.

Another embodiment comprises cyclosporin A, Hetoxol TD-18®, and Maize oil.

Another embodiment comprises cyclosporin A, Hetoxol TD-3®, and Maize oil.

Another embodiment comprises cyclosporin A, Hetoxol TD-6®, and Maize oil.

Another embodiment comprises cyclosporin A, Hetoxol TD-9®, and Maize oil.

Another embodiment comprises cyclosporin A, Hetoxol TDEP-15®, and Maize oil.

Another embodiment comprises cyclosporin A, Hetoxol TDEP-63®, and Maize oil.

Another embodiment comprises cyclosporin A, Iconol 24-12®, and Maize oil.

Another embodiment comprises cyclosporin A, Iconol 24-23®, and Maize oil.

Another embodiment comprises cyclosporin A, Iconol 24-4®, and Maize oil.

Another embodiment comprises cyclosporin A, Iconol DA-4®, and Maize oil.

Another embodiment comprises cyclosporin A, Iconol DA-6®, and Maize oil.

Another embodiment comprises cyclosporin A, Iconol TDA-10®, and Maize oil.

Another embodiment comprises cyclosporin A, Iconol TDA-3®, and Maize oil.

Another embodiment comprises cyclosporin A, Iconol TDA-6®, and Maize oil.

Another embodiment comprises cyclosporin A, Iconol TDA-8-90%®, and Maize oil.

Another embodiment comprises cyclosporin A, Iconol TDA-9®, and Maize oil.

Another embodiment comprises cyclosporin A, Igepal CA-210®, and Maize oil.

Another embodiment comprises cyclosporin A, Igepal CA-407®, and Maize oil.

Another embodiment comprises cyclosporin A, Igepal CA-420®, and Maize oil.

Another embodiment comprises cyclosporin A, Igepal CA-520®, and Maize oil.

Another embodiment comprises cyclosporin A, Igepal CA-620®, and Maize oil.

Another embodiment comprises cyclosporin A, Igepal CA-630®, and Maize oil.

Another embodiment comprises cyclosporin A, Igepal CA-720®, and Maize oil.

Another embodiment comprises cyclosporin A, Igepal CA-877®, and Maize oil.

Another embodiment comprises cyclosporin A, Igepal CA-887®, and Maize oil.

Another embodiment comprises cyclosporin A, Igepal CA-897®, and Maize oil.

Another embodiment comprises cyclosporin A, Igepal CO-210®, and Maize oil.

Another embodiment comprises cyclosporin A, Igepal CO-430®, and Maize oil.

Another embodiment comprises cyclosporin A, Igepal CO-520®, and Maize oil.

Another embodiment comprises cyclosporin A, Igepal CO-530®, and Maize oil.

Another embodiment comprises cyclosporin A, Igepal CO-610®, and Maize oil.

Another embodiment comprises cyclosporin A, Igepal CO-630®, and Maize oil.

Another embodiment comprises cyclosporin A, Igepal CO-630 Special®, and Maize oil.

Another embodiment comprises cyclosporin A, Igepal CO-660®, and Maize oil.

Another embodiment comprises cyclosporin A, Igepal CO-710®, and Maize oil.

Another embodiment comprises cyclosporin A, Igepal CO-720®, and Maize oil.

Another embodiment comprises cyclosporin A, Igepal CO-730®, and Maize oil.

Another embodiment comprises cyclosporin A, Igepal CO-738®, and Maize oil.

Another embodiment comprises cyclosporin A, Igepal CO-850®, and Maize oil.

Another embodiment comprises cyclosporin A, Igepal CO-880®, and Maize oil.

Another embodiment comprises cyclosporin A, Igepal CO-887®, and Maize oil.

Another embodiment comprises cyclosporin A, Igepal CO-890®, and Maize oil.

Another embodiment comprises cyclosporin A, Igepal CO-897®, and Maize oil.

Another embodiment comprises cyclosporin A, Igepal CO-970®, and Maize oil.

Another embodiment comprises cyclosporin A, Igepal CO-977®, and Maize oil.

Another embodiment comprises cyclosporin A, Igepal CO-987®, and Maize oil.

Another embodiment comprises cyclosporin A, Igepal CO-990 FLK®, and Maize oil.

Another embodiment comprises cyclosporin A, Igepal CO-997®, and Maize oil.

Another embodiment comprises cyclosporin A, Igepal DM-430®, and Maize oil.

Another embodiment comprises cyclosporin A, Igepal DM-530®, and Maize oil.

Another embodiment comprises cyclosporin A, Igepal DM-710®, and Maize oil.

Another embodiment comprises cyclosporin A, Igepal DM-730®, and Maize oil.

Another embodiment comprises cyclosporin A, Igepal DM-880®, and Maize oil.

Another embodiment comprises cyclosporin A, Igepal DM-970 FLK®, and Maize oil.

Another embodiment comprises cyclosporin A, Igepal OD-410®, and Maize oil.

Another embodiment comprises cyclosporin A, Igepal RC-520®, and Maize oil.

Another embodiment comprises cyclosporin A, Igepal RC-620®, and Maize oil.

Another embodiment comprises cyclosporin A, Igepal RC-630®, and Maize oil.

Another embodiment comprises cyclosporin A, Lexemul CS-20®, and Maize oil.

Another embodiment comprises cyclosporin A, Lipo GMS 450®, and Maize oil.

Another embodiment comprises cyclosporin A, Lipo Polyglycol 1000®, and Maize oil.

Another embodiment comprises cyclosporin A, Lipo Polyglycol 200®, and Maize oil.

Another embodiment comprises cyclosporin A, Lipo Polyglycol 300®, and Maize oil.

Another embodiment comprises cyclosporin A, Lipo Polyglycol 3350®, and Maize oil.

Another embodiment comprises cyclosporin A, Lipo Polyglycol 400®, and Maize oil.

Another embodiment comprises cyclosporin A, Lipo Polyglycol 600®, and Maize oil.

Another embodiment comprises cyclosporin A, Lipocol C-10®, and Maize oil.

Another embodiment comprises cyclosporin A, Lipocol C-2®, and Maize oil.

Another embodiment comprises cyclosporin A, Lipocol C-20®, and Maize oil.

Another embodiment comprises cyclosporin A, Lipocol L-12®, and Maize oil.

Another embodiment comprises cyclosporin A, Lipocol L-23®, and Maize oil.

Another embodiment comprises cyclosporin A, Lipocol L-4®, and Maize oil.

Another embodiment comprises cyclosporin A, Lipocol O-10®, and Maize oil.

Another embodiment comprises cyclosporin A, Lipocol O-2®, and Maize oil.

Another embodiment comprises cyclosporin A, Lipocol O-20®, and Maize oil.

Another embodiment comprises cyclosporin A, Lipocol O-5®, and Maize oil.

Another embodiment comprises cyclosporin A, Lipocol S-10®, and Maize oil.

Another embodiment comprises cyclosporin A, Lipocol S-2®, and Maize oil.

Another embodiment comprises cyclosporin A, Lipocol S-20®, and Maize oil.

Another embodiment comprises cyclosporin A, Lipocol SC-15®, and Maize oil.

Another embodiment comprises cyclosporin A, Lipocol SC-20®, and Maize oil.

Another embodiment comprises cyclosporin A, Lipocol SC-4®, and Maize oil.

Another embodiment comprises cyclosporin A, Lipocol TD-12®, and Maize oil.

Another embodiment comprises cyclosporin A, Lumulse 40-O K®, and Maize oil.

Another embodiment comprises cyclosporin A, Lumulse 42-O K®, and Maize oil.

Another embodiment comprises cyclosporin A, Lumulse GMT-40®, and Maize oil.

Another embodiment comprises cyclosporin A, Lumulse PEG 1450®, and Maize oil.

Another embodiment comprises cyclosporin A, Lumulse PEG 1450 NF®, and Maize oil.

Another embodiment comprises cyclosporin A, Lumulse PEG 8000®, and Maize oil.

Another embodiment comprises cyclosporin A, Lumulse PGO®, and Maize oil.

Another embodiment comprises cyclosporin A, Lumulse POE (2) Oleyl Amine®, and Maize oil.

Another embodiment comprises cyclosporin A, Magrabar PGO-104®, and Maize oil.

Another embodiment comprises cyclosporin A, Mapeg 200 ML®, and Maize oil.

Another embodiment comprises cyclosporin A, Mapeg 400 MO®, and Maize oil.

Another embodiment comprises cyclosporin A, Mapeg 400 MOT®, and Maize oil.

Another embodiment comprises cyclosporin A, Mapeg 400DO®, and Maize oil.

Another embodiment comprises cyclosporin A, Mapeg 400DOT®, and Maize oil.

Another embodiment comprises cyclosporin A, Mapeg 600DOT®, and Maize oil.

Another embodiment comprises cyclosporin A, Marlox B 24/80®, and Maize oil.

Another embodiment comprises cyclosporin A, Marlox FK 86®, and Maize oil.

Another embodiment comprises cyclosporin A, Marlox MO 154®, and Maize oil.

Another embodiment comprises cyclosporin A, Mazox CDA CG®, and Maize oil.

Another embodiment comprises cyclosporin A, Monoglycerides Glyceryl Monestearate®, and Maize oil.

Another embodiment comprises cyclosporin A, Myrj 45®, and Maize oil.

Another embodiment comprises cyclosporin A, Myrj 52®, and Maize oil.

Another embodiment comprises cyclosporin A, Myrj 52S®, and Maize oil.

Another embodiment comprises cyclosporin A, Myrj 53®, and Maize oil.

Another embodiment comprises cyclosporin A, Myrj 59®, and Maize oil.

Another embodiment comprises cyclosporin A, Norfox NP-12®, and Maize oil.

Another embodiment comprises cyclosporin A, Norfox NP-4®, and Maize oil.

Another embodiment comprises cyclosporin A, Norfox NP-6®, and Maize oil.

Another embodiment comprises cyclosporin A, Norfox NP-887®, and Maize oil.

Another embodiment comprises cyclosporin A, Norfox NP-9®, and Maize oil.

Another embodiment comprises cyclosporin A, Norfox NP-977®, and Maize oil.

Another embodiment comprises cyclosporin A, Norfox OP-100®, and Maize oil.

Another embodiment comprises cyclosporin A, Norfox OP-102®, and Maize oil.

Another embodiment comprises cyclosporin A, Norfox OP-114®, and Maize oil.

Another embodiment comprises cyclosporin A, Norfox OP-45®, and Maize oil.

Another embodiment comprises cyclosporin A, Pegosperse 100-L®, and Maize oil.

Another embodiment comprises cyclosporin A, Pegosperse 100-O®, and Maize oil.

Another embodiment comprises cyclosporin A, Pegosperse 100-S®, and Maize oil.

Another embodiment comprises cyclosporin A, Pegosperse 1500-MS®, and Maize oil.

Another embodiment comprises cyclosporin A, Pegosperse 1750-MS®, and Maize oil.

Another embodiment comprises cyclosporin A, Pegosperse 200 DL®, and Maize oil.

Another embodiment comprises cyclosporin A, Pegosperse 200-ML®, and Maize oil.

Another embodiment comprises cyclosporin A, Pegosperse 400-DL®, and Maize oil.

Another embodiment comprises cyclosporin A, Pegosperse 400-DO®, and Maize oil.

Another embodiment comprises cyclosporin A, Pegosperse 400-DS®, and Maize oil.

Another embodiment comprises cyclosporin A, Pegosperse 400-ML®, and Maize oil.

Another embodiment comprises cyclosporin A, Pegosperse 400-MO®, and Maize oil.

Another embodiment comprises cyclosporin A, Pegosperse 400-MOT®, and Maize oil.

Another embodiment comprises cyclosporin A, Pegosperse 400-MS®, and Maize oil.

Another embodiment comprises cyclosporin A, Pegosperse 50-DS®, and Maize oil.

Another embodiment comprises cyclosporin A, Pegosperse 50-MS®, and Maize oil.

Another embodiment comprises cyclosporin A, Pegosperse 600-ML®, and Maize oil.

Another embodiment comprises cyclosporin A, Pegosperse 600-MS®, and Maize oil.

Another embodiment comprises cyclosporin A, Plurafac A-38®, and Maize oil.

Another embodiment comprises cyclosporin A, Plurafac B-25-5®, and Maize oil.

Another embodiment comprises cyclosporin A, Plurafac B-26®, and Maize oil.

Another embodiment comprises cyclosporin A, Plurafac D-25®, and Maize oil.

Another embodiment comprises cyclosporin A, Plurafac LF 131®, and Maize oil.

Another embodiment comprises cyclosporin A, Plurafac LF 4030®, and Maize oil.

Another embodiment comprises cyclosporin A, Plurafac LF 7000®, and Maize oil.

Another embodiment comprises cyclosporin A, Plurafac RA-20®, and Maize oil.

Another embodiment comprises cyclosporin A, Plurafac RA-30®, and Maize oil.

Another embodiment comprises cyclosporin A, Plurafac RA-40®, and Maize oil.

Another embodiment comprises cyclosporin A, Pluronic 10R5®, and Maize oil.

Another embodiment comprises cyclosporin A, Pluronic 17R2®, and Maize oil.

Another embodiment comprises cyclosporin A, Pluronic 17R4®, and Maize oil.

Another embodiment comprises cyclosporin A, Pluronic 25R2®, and Maize oil.

Another embodiment comprises cyclosporin A, Pluronic 25R4®, and Maize oil.

Another embodiment comprises cyclosporin A, Pluronic 31R1®, and Maize oil.

Another embodiment comprises cyclosporin A, Pluronic F108®, and Maize oil.

Another embodiment comprises cyclosporin A, Pluronic F127®, and Maize oil.

Another embodiment comprises cyclosporin A, Pluronic F38®, and Maize oil.

Another embodiment comprises cyclosporin A, Pluronic F68®, and Maize oil.

Another embodiment comprises cyclosporin A, Pluronic F68LF®, and Maize oil.

Another embodiment comprises cyclosporin A, Pluronic F77®, and Maize oil.

Another embodiment comprises cyclosporin A, Pluronic F87®, and Maize oil.

Another embodiment comprises cyclosporin A, Pluronic F88®, and Maize oil.

Another embodiment comprises cyclosporin A, Pluronic F98®, and Maize oil.

Another embodiment comprises cyclosporin A, Pluronic L10®, and Maize oil.

Another embodiment comprises cyclosporin A, Pluronic L101®, and Maize oil.

Another embodiment comprises cyclosporin A, Pluronic L121®, and Maize oil.

Another embodiment comprises cyclosporin A, Pluronic L31®, and Maize oil.

Another embodiment comprises cyclosporin A, Pluronic L35®, and Maize oil.

Another embodiment comprises cyclosporin A, Pluronic L43®, and Maize oil.

Another embodiment comprises cyclosporin A, Pluronic L44®, and Maize oil.

Another embodiment comprises cyclosporin A, Pluronic L61®, and Maize oil.

Another embodiment comprises cyclosporin A, Pluronic L62®, and Maize oil.

Another embodiment comprises cyclosporin A, Pluronic L62D®, and Maize oil.

Another embodiment comprises cyclosporin A, Pluronic L62LF®, and Maize oil.

Another embodiment comprises cyclosporin A, Pluronic L64®, and Maize oil.

Another embodiment comprises cyclosporin A, Pluronic L81®, and Maize oil.

Another embodiment comprises cyclosporin A, Pluronic L92®, and Maize oil.

Another embodiment comprises cyclosporin A, Pluronic P103®, and Maize oil.

Another embodiment comprises cyclosporin A, Pluronic P104®, and Maize oil.

Another embodiment comprises cyclosporin A, Pluronic P105®, and Maize oil.

Another embodiment comprises cyclosporin A, Pluronic P123®, and Maize oil.

Another embodiment comprises cyclosporin A, Pluronic P65®, and Maize oil.

Another embodiment comprises cyclosporin A, Pluronic P84®, and Maize oil.

Another embodiment comprises cyclosporin A, Pluronic P85®, and Maize oil.

Another embodiment comprises cyclosporin A, Polyaldo 10-2-P®, and Maize oil.

Another embodiment comprises cyclosporin A, Polyaldo DGDO®, and Maize oil.

Another embodiment comprises cyclosporin A, Polyaldo HGDS®, and Maize oil.

Another embodiment comprises cyclosporin A, Polyaldo TGMS®, and Maize oil.

Another embodiment comprises cyclosporin A, Protegin V®, and Maize oil.

Another embodiment comprises cyclosporin A, Protegin W®, and Maize oil.

Another embodiment comprises cyclosporin A, Protegin WX®, and Maize oil.

Another embodiment comprises cyclosporin A, Protegin XV®, and Maize oil.

Another embodiment comprises cyclosporin A, Rhodasurf BC-420®, and Maize oil.

Another embodiment comprises cyclosporin A, Rhodasurf BC-610®, and Maize oil.

Another embodiment comprises cyclosporin A, Rhodasurf BC-630®, and Maize oil.

Another embodiment comprises cyclosporin A, Rhodasurf BC-720®, and Maize oil.

Another embodiment comprises cyclosporin A, Rhodasurf BC-840®, and Maize oil.

Another embodiment comprises cyclosporin A, Rhodasurf L 1®, and Maize oil.

Another embodiment comprises cyclosporin A, Rhodasurf L 12®, and Maize oil.

Another embodiment comprises cyclosporin A, Rhodasurf L 2®, and Maize oil.

Another embodiment comprises cyclosporin A, Rhodasurf L 20®, and Maize oil.

Another embodiment comprises cyclosporin A, Rhodasurf L 3®, and Maize oil.

Another embodiment comprises cyclosporin A, Rhodasurf L 30®, and Maize oil.

Another embodiment comprises cyclosporin A, Rhodasurf L 4®, and Maize oil.

Another embodiment comprises cyclosporin A, Rhodasurf L 50®, and Maize oil.

Another embodiment comprises cyclosporin A, Rhodasurf L 7/90®, and Maize oil.

Another embodiment comprises cyclosporin A, Rhodasurf LA-3®, and Maize oil.

Another embodiment comprises cyclosporin A, Rhodasurf LA-7®, and Maize oil.

Another embodiment comprises cyclosporin A, Rhodasurf LAN-23®, and Maize oil.

Another embodiment comprises cyclosporin A, Rhodasurf ON-870®, and Maize oil.

Another embodiment comprises cyclosporin A, Rhodasurf ON-877®, and Maize oil.

Another embodiment comprises cyclosporin A, Rhodasurf TDA-8/5®, and Maize oil.

Another embodiment comprises cyclosporin A, Ritapro 100®, and Maize oil.

Another embodiment comprises cyclosporin A, Ritapro 165®, and Maize oil.

Another embodiment comprises cyclosporin A, Ritapro 200®, and Maize oil.

Another embodiment comprises cyclosporin A, Ritapro 300®, and Maize oil.

Another embodiment comprises cyclosporin A, Ritoleth 10®, and Maize oil.

Another embodiment comprises cyclosporin A, Ritoleth 2®, and Maize oil.

Another embodiment comprises cyclosporin A, Ritoleth 20®, and Maize oil.

Another embodiment comprises cyclosporin A, Ritoleth 5®, and Maize oil.

Another embodiment comprises cyclosporin A, Santone 10-10-0®, and Maize oil.

Another embodiment comprises cyclosporin A, Santone 3-1-S XTR®, and Maize oil.

Another embodiment comprises cyclosporin A, Santone 3-1-SH®, and Maize oil.

Another embodiment comprises cyclosporin A, Santone 8-1-O®, and Maize oil.

Another embodiment comprises cyclosporin A, Solulan 16®, and Maize oil.

Another embodiment comprises cyclosporin A, Solulan 75®, and Maize oil.

Another embodiment comprises cyclosporin A, Solulan C-24®, and Maize oil.

Another embodiment comprises cyclosporin A, Solulan L-575®, and Maize oil.

Another embodiment comprises cyclosporin A, Standamox CAW®, and Maize oil.

Another embodiment comprises cyclosporin A, Surfine AZI-A®, and Maize oil.

Another embodiment comprises cyclosporin A, Surfonic CO-15®, and Maize oil.

Another embodiment comprises cyclosporin A, Surfonic CO-25®, and Maize oil.

Another embodiment comprises cyclosporin A, Surfonic CO-30®, and Maize oil.

Another embodiment comprises cyclosporin A, Surfonic CO-36®, and Maize oil.

Another embodiment comprises cyclosporin A, Surfonic CO-42®, and Maize oil.

Another embodiment comprises cyclosporin A, Surfonic E400-MO®, and Maize oil.

Another embodiment comprises cyclosporin A, Surfonic L12-3®, and Maize oil.

Another embodiment comprises cyclosporin A, Surfonic L12-6®, and Maize oil.

Another embodiment comprises cyclosporin A, Surfonic L12-8®, and Maize oil.

Another embodiment comprises cyclosporin A, Surfonic L24-12®, and Maize oil.

Another embodiment comprises cyclosporin A, Surfonic L24-2®, and Maize oil.

Another embodiment comprises cyclosporin A, Surfonic L24-22®, and Maize oil.

Another embodiment comprises cyclosporin A, Surfonic L24-3®, and Maize oil.

Another embodiment comprises cyclosporin A, Surfonic L24-4®, and Maize oil.

Another embodiment comprises cyclosporin A, Surfonic L24-5®, and Maize oil.

Another embodiment comprises cyclosporin A, Surfonic L24-7®, and Maize oil.

Another embodiment comprises cyclosporin A, Surfonic L24-7.1®, and Maize oil.

Another embodiment comprises cyclosporin A, Surfonic L24-9®, and Maize oil.

Another embodiment comprises cyclosporin A, Surfonic LF-17®, and Maize oil.

Another embodiment comprises cyclosporin A, Surfonic LF-18®, and Maize oil.

Another embodiment comprises cyclosporin A, Surfonic LF-37®, and Maize oil.

Another embodiment comprises cyclosporin A, Surfonic LF-41®, and Maize oil.

Another embodiment comprises cyclosporin A, Surfonic LF-42®, and Maize oil.

Another embodiment comprises cyclosporin A, Surfonic LF-47®, and Maize oil.

Another embodiment comprises cyclosporin A, Surfonic LF-68®, and Maize oil.

Another embodiment comprises cyclosporin A, Surfonic N-10®, and Maize oil.

Another embodiment comprises cyclosporin A, Surfonic N-100®, and Maize oil.

Another embodiment comprises cyclosporin A, Surfonic N-102®, and Maize oil.

Another embodiment comprises cyclosporin A, Surfonic N-120®, and Maize oil.

Another embodiment comprises cyclosporin A, Surfonic N-150®, and Maize oil.

Another embodiment comprises cyclosporin A, Surfonic N-200®, and Maize oil.

Another embodiment comprises cyclosporin A, Surfonic N-300®, and Maize oil.

Another embodiment comprises cyclosporin A, Surfonic N-31.5®, and Maize oil.

Another embodiment comprises cyclosporin A, Surfonic N-40®, and Maize oil.

Another embodiment comprises cyclosporin A, Surfonic N-400®, and Maize oil.

Another embodiment comprises cyclosporin A, Surfonic N-60®, and Maize oil.

Another embodiment comprises cyclosporin A, Surfonic N-85®, and Maize oil.

Another embodiment comprises cyclosporin A, Surfonic N-95®, and Maize oil.

Another embodiment comprises cyclosporin A, Surfonic NB-18®, and Maize oil.

Another embodiment comprises cyclosporin A, Surfonic NB-23®, and Maize oil.

Another embodiment comprises cyclosporin A, Surfonic NB-25®, and Maize oil.

Another embodiment comprises cyclosporin A, Surfonic NB-307®, and Maize oil.

Another embodiment comprises cyclosporin A, Surfonic NB-31®, and Maize oil.

Another embodiment comprises cyclosporin A, Surfonic NB-41®, and Maize oil.

Another embodiment comprises cyclosporin A, Surfonic NB-43®, and Maize oil.

Another embodiment comprises cyclosporin A, Surfonic OP-100®, and Maize oil.

Another embodiment comprises cyclosporin A, Surfonic OP-120®, and Maize oil.

Another embodiment comprises cyclosporin A, Surfonic OP-15®, and Maize oil.

Another embodiment comprises cyclosporin A, Surfonic OP-35®, and Maize oil.

Another embodiment comprises cyclosporin A, Surfonic OP-70®, and Maize oil.

Another embodiment comprises cyclosporin A, Surfonic P1®, and Maize oil.

Another embodiment comprises cyclosporin A, Surfonic P3®, and Maize oil.

Another embodiment comprises cyclosporin A, Surfonic P5®, and Maize oil.

Another embodiment comprises cyclosporin A, Surfonic P6®, and Maize oil.

Another embodiment comprises cyclosporin A, Surfonic POA-17R2®, and Maize oil.

Another embodiment comprises cyclosporin A, Surfonic POA-25R2®, and Maize oil.

Another embodiment comprises cyclosporin A, Surfonic TDA-11®, and Maize oil.

Another embodiment comprises cyclosporin A, Surfonic TDA-3B®, and Maize oil.

Another embodiment comprises cyclosporin A, Surfonic TDA-6®, and Maize oil.

Another embodiment comprises cyclosporin A, Surfonic TDA-6/88®, and Maize oil.

Another embodiment comprises cyclosporin A, Surfonic TDA-8®, and Maize oil.

Another embodiment comprises cyclosporin A, Surfonic TDA-8/90®, and Maize oil.

Another embodiment comprises cyclosporin A, Surfonic TDA-9®, and Maize oil.

Another embodiment comprises cyclosporin A, Surfonic TX-CCN®, and Maize oil.

Another embodiment comprises cyclosporin A, Tagat CH 40®, and Maize oil.

Another embodiment comprises cyclosporin A, Tagat CH 60®, and Maize oil.

Another embodiment comprises cyclosporin A, Tagat L 2®, and Maize oil.

Another embodiment comprises cyclosporin A, Tagat S®, and Maize oil.

Another embodiment comprises cyclosporin A, Tagat S 2®, and Maize oil.

Another embodiment comprises cyclosporin A, Tally 100 Plus®, and Maize oil.

Another embodiment comprises cyclosporin A, T-Det A106®, and Maize oil.

Another embodiment comprises cyclosporin A, T-Det A109®, and Maize oil.

Another embodiment comprises cyclosporin A, T-Det A136®, and Maize oil.

Another embodiment comprises cyclosporin A, T-Det A139®, and Maize oil.

Another embodiment comprises cyclosporin A, T-Det A2412®, and Maize oil.

Another embodiment comprises cyclosporin A, T-Det A243®, and Maize oil.

Another embodiment comprises cyclosporin A, T-Det A247®, and Maize oil.

Another embodiment comprises cyclosporin A, T-Det A249®, and Maize oil.

Another embodiment comprises cyclosporin A, T-Det A467®, and Maize oil.

Another embodiment comprises cyclosporin A, T-Det COE®, and Maize oil.

Another embodiment comprises cyclosporin A, T-Det DD-10®, and Maize oil.

Another embodiment comprises cyclosporin A, T-Det DD-5®, and Maize oil.

Another embodiment comprises cyclosporin A, T-Det DD-7®, and Maize oil.

Another embodiment comprises cyclosporin A, T-Det EPO-104®, and Maize oil.

Another embodiment comprises cyclosporin A, T-Det EPO-61®, and Maize oil.

Another embodiment comprises cyclosporin A, T-Det EPO-62®, and Maize oil.

Another embodiment comprises cyclosporin A, T-Det EPO-64®, and Maize oil.

Another embodiment comprises cyclosporin A, T-Det N-1.5®, and Maize oil.

Another embodiment comprises cyclosporin A, T-Det N-10.5®, and Maize oil.

Another embodiment comprises cyclosporin A, T-Det N-100®, and Maize oil.

Another embodiment comprises cyclosporin A, T-Det N-1007®, and Maize oil.

Another embodiment comprises cyclosporin A, T-Det N-12®, and Maize oil.

Another embodiment comprises cyclosporin A, T-Det N-14®, and Maize oil.

Another embodiment comprises cyclosporin A, T-Det N-20®, and Maize oil.

Another embodiment comprises cyclosporin A, T-Det N-30®, and Maize oil.

Another embodiment comprises cyclosporin A, T-Det N-307®, and Maize oil.

Another embodiment comprises cyclosporin A, T-Det N-4®, and Maize oil.

Another embodiment comprises cyclosporin A, T-Det N-40®, and Maize oil.

Another embodiment comprises cyclosporin A, T-Det N-407®, and Maize oil.

Another embodiment comprises cyclosporin A, T-Det N-50®, and Maize oil.

Another embodiment comprises cyclosporin A, T-Det N-507®, and Maize oil.

Another embodiment comprises cyclosporin A, T-Det N-6®, and Maize oil.

Another embodiment comprises cyclosporin A, T-Det N-70®, and Maize oil.

Another embodiment comprises cyclosporin A, T-Det N-8®, and Maize oil.

Another embodiment comprises cyclosporin A, T-Det N-9.5®, and Maize oil.

Another embodiment comprises cyclosporin A, T-Det O-12®, and Maize oil.

Another embodiment comprises cyclosporin A, T-Det O-165®, and Maize oil.

Another embodiment comprises cyclosporin A, T-Det O-307®, and Maize oil.

Another embodiment comprises cyclosporin A, T-Det O-40®, and Maize oil.

Another embodiment comprises cyclosporin A, T-Det O-407®, and Maize oil.

Another embodiment comprises cyclosporin A, T-Det O-6®, and Maize oil.

Another embodiment comprises cyclosporin A, T-Det O-8®, and Maize oil.

Another embodiment comprises cyclosporin A, T-Det O-9®, and Maize oil.

Another embodiment comprises cyclosporin A, Tegin®, and Maize oil.

Another embodiment comprises cyclosporin A, Tergitol 15-S-12®, and Maize oil.

Another embodiment comprises cyclosporin A, Tergitol 15-S-15®, and Maize oil.

Another embodiment comprises cyclosporin A, Tergitol 15-S-20®, and Maize oil.

Another embodiment comprises cyclosporin A, Tergitol 15-S-20 (80AQ)®, and Maize oil.

Another embodiment comprises cyclosporin A, Tergitol 15-S-3®, and Maize oil.

Another embodiment comprises cyclosporin A, Tergitol 15-S-30®, and Maize oil.

Another embodiment comprises cyclosporin A, Tergitol 15-S-40®, and Maize oil.

Another embodiment comprises cyclosporin A, Tergitol 15-S-40 (70AQ)®, and Maize oil.

Another embodiment comprises cyclosporin A, Tergitol 15-S-5®, and Maize oil.

Another embodiment comprises cyclosporin A, Tergitol 15-S-7®, and Maize oil.

Another embodiment comprises cyclosporin A, Tergitol 15-S-9®, and Maize oil.

Another embodiment comprises cyclosporin A, Tergitol L-101®, and Maize oil.

Another embodiment comprises cyclosporin A, Tergitol L-61®, and Maize oil.

Another embodiment comprises cyclosporin A, Tergitol L-62®, and Maize oil.

Another embodiment comprises cyclosporin A, Tergitol L-64®, and Maize oil.

Another embodiment comprises cyclosporin A, Tergitol L-81®, and Maize oil.

Another embodiment comprises cyclosporin A, Tergitol NP-10®, and Maize oil.

Another embodiment comprises cyclosporin A, Tergitol NP-11®, and Maize oil.

Another embodiment comprises cyclosporin A, Tergitol NP-12®, and Maize oil.

Another embodiment comprises cyclosporin A, Tergitol NP-13®, and Maize oil.

Another embodiment comprises cyclosporin A, Tergitol NP-15®, and Maize oil.

Another embodiment comprises cyclosporin A, Tergitol NP-30®, and Maize oil.

Another embodiment comprises cyclosporin A, Tergitol NP-30 (70 AQ)®, and Maize oil.

Another embodiment comprises cyclosporin A, Tergitol NP-4®, and Maize oil.

Another embodiment comprises cyclosporin A, Tergitol NP-40®, and Maize oil.

Another embodiment comprises cyclosporin A, Tergitol NP-40 (70 AQ)®, and Maize oil.

Another embodiment comprises cyclosporin A, Tergitol NP-50 (70 AQ)®, and Maize oil.

Another embodiment comprises cyclosporin A, Tergitol NP-55 (70 AQ)®, and Maize oil.

Another embodiment comprises cyclosporin A, Tergitol NP-6®, and Maize oil.

Another embodiment comprises cyclosporin A, Tergitol NP-7®, and Maize oil.

Another embodiment comprises cyclosporin A, Tergitol NP-70 (70 AQ)®, and Maize oil.

Another embodiment comprises cyclosporin A, Tergitol NP-8®, and Maize oil.

Another embodiment comprises cyclosporin A, Tergitol NP-9®, and Maize oil.

Another embodiment comprises cyclosporin A, Tergitol NP-9.5®, and Maize oil.

Another embodiment comprises cyclosporin A, Tergitol TMN-10®, and Maize oil.

Another embodiment comprises cyclosporin A, Tergitol TMN-3®, and Maize oil.

Another embodiment comprises cyclosporin A, Tergitol TMN-6®, and Maize oil.

Another embodiment comprises cyclosporin A, Tergitol XD®, and Maize oil.

Another embodiment comprises cyclosporin A, Tergitol XH®, and Maize oil.

Another embodiment comprises cyclosporin A, Tergitol XJ®, and Maize oil.

Another embodiment comprises cyclosporin A, Tetronic 1107®, and Maize oil.

Another embodiment comprises cyclosporin A, Tetronic 1307®, and Maize oil.

Another embodiment comprises cyclosporin A, Tetronic 150R1®, and Maize oil.

Another embodiment comprises cyclosporin A, Tetronic 304®, and Maize oil.

Another embodiment comprises cyclosporin A, Tetronic 701®, and Maize oil.

Another embodiment comprises cyclosporin A, Tetronic 901®, and Maize oil.

Another embodiment comprises cyclosporin A, Tetronic 904®, and Maize oil.

Another embodiment comprises cyclosporin A, Tetronic 908®, and Maize oil.

Another embodiment comprises cyclosporin A, Tetronic 90R4®, and Maize oil.

Another embodiment comprises cyclosporin A, Tomadol 1-3®, and Maize oil.

Another embodiment comprises cyclosporin A, Tomadol 1-5®, and Maize oil.

Another embodiment comprises cyclosporin A, Tomadol 1-7®, and Maize oil.

Another embodiment comprises cyclosporin A, Tomadol 23-1®, and Maize oil.

Another embodiment comprises cyclosporin A, Tomadol 23-3®, and Maize oil.

Another embodiment comprises cyclosporin A, Tomadol 23-5®, and Maize oil.

Another embodiment comprises cyclosporin A, Tomadol 23-6.5®, and Maize oil.

Another embodiment comprises cyclosporin A, Tomadol 25-12®, and Maize oil.

Another embodiment comprises cyclosporin A, Tomadol 25-3®, and Maize oil.

Another embodiment comprises cyclosporin A, Tomadol 25-7®, and Maize oil.

Another embodiment comprises cyclosporin A, Tomadol 25-9®, and Maize oil.

Another embodiment comprises cyclosporin A, Tomadol 45-13®, and Maize oil.

Another embodiment comprises cyclosporin A, Tomadol 45-2.5®, and Maize oil.

Another embodiment comprises cyclosporin A, Tomadol 45-7®, and Maize oil.

Another embodiment comprises cyclosporin A, Tomadol 91-2.5®, and Maize oil.

Another embodiment comprises cyclosporin A, Tomadol 91-6®, and Maize oil.

Another embodiment comprises cyclosporin A, Tomadol 91-8®, and Maize oil.

Another embodiment comprises cyclosporin A, Triton X-100®, and Maize oil.

Another embodiment comprises cyclosporin A, Triton X-102®, and Maize oil.

Another embodiment comprises cyclosporin A, Triton X-114®, and Maize oil.

Another embodiment comprises cyclosporin A, Triton X-15®, and Maize oil.

Another embodiment comprises cyclosporin A, Triton X-165 (70%)®, and Maize oil.

Another embodiment comprises cyclosporin A, Triton X-207®, and Maize oil.

Another embodiment comprises cyclosporin A, Triton X-305 (70%)®, and Maize oil.

Another embodiment comprises cyclosporin A, Triton X-35®, and Maize oil.

Another embodiment comprises cyclosporin A, Triton X-405 (70%)®, and Maize oil.

Another embodiment comprises cyclosporin A, Triton X-45®, and Maize oil.

Another embodiment comprises cyclosporin A, Triton X-705 (70%)®, and Maize oil.

Another embodiment comprises cyclosporin A, Volpo S-10®, and Maize oil.

Another embodiment comprises cyclosporin A, Volpo S-2®, and Maize oil.

Another embodiment comprises cyclosporin A, Volpo S-20®, and Maize oil.

Another embodiment comprises cyclosporin A, Volpo-10®, and Maize oil.

Another embodiment comprises cyclosporin A, Volpo-20®, and Maize oil.

Another embodiment comprises cyclosporin A, Volpo-3®, and Maize oil.

Another embodiment comprises cyclosporin A, Volpo-5®, and Maize oil.

Another embodiment comprises cyclosporin A, Abex 2515®, and Linseed oil.

Another embodiment comprises cyclosporin A, Abex 2525/40®, and Linseed oil.

Another embodiment comprises cyclosporin A, Abex 2535®, and Linseed oil.

Another embodiment comprises cyclosporin A, Abex 2545®, and Linseed oil.

Another embodiment comprises cyclosporin A, Acconon E®, and Linseed oil.

Another embodiment comprises cyclosporin A, Alfonic 1216CO-9®, and Linseed oil.

Another embodiment comprises cyclosporin A, Alfonic 1412-3®, and Linseed oil.

Another embodiment comprises cyclosporin A, Alfonic TDA-12®, and Linseed oil.

Another embodiment comprises cyclosporin A, Alfonic TDA-3®, and Linseed oil.

Another embodiment comprises cyclosporin A, Alfonic TDA-4®, and Linseed oil.

Another embodiment comprises cyclosporin A, Alfonic TDA-6®, and Linseed oil.

Another embodiment comprises cyclosporin A, Alfonic TDA-7®, and Linseed oil.

Another embodiment comprises cyclosporin A, Alfonic TDA-8®, and Linseed oil.

Another embodiment comprises cyclosporin A, Alfonic TDA-9®, and Linseed oil.

Another embodiment comprises cyclosporin A, Alkamuls 400-DO®, and Linseed oil.

Another embodiment comprises cyclosporin A, Alkamuls 400-MO/E®, and Linseed oil.

Another embodiment comprises cyclosporin A, Ameroxol OE-10®, and Linseed oil.

Another embodiment comprises cyclosporin A, Ameroxol OE-2®, and Linseed oil.

Another embodiment comprises cyclosporin A, Ameroxol OE-20®, and Linseed oil.

Another embodiment comprises cyclosporin A, Armix 176®, and Linseed oil.

Another embodiment comprises cyclosporin A, Armix 180-C®, and Linseed oil.

Another embodiment comprises cyclosporin A, Armix 183®, and Linseed oil.

Another embodiment comprises cyclosporin A, Brij 35®, and Linseed oil.

Another embodiment comprises cyclosporin A, Brij 35 Liquid®, and Linseed oil.

Another embodiment comprises cyclosporin A, Brij 35 SP®, and Linseed oil.

Another embodiment comprises cyclosporin A, Canasol MJ 2109®, and Linseed oil.

Another embodiment comprises cyclosporin A, Canasol MJ 52®, and Linseed oil.

Another embodiment comprises cyclosporin A, Canasol R 3600®, and Linseed oil.

Another embodiment comprises cyclosporin A, Canasol R 3603®, and Linseed oil.

Another embodiment comprises cyclosporin A, Canasol R 4000 H®, and Linseed oil.

Another embodiment comprises cyclosporin A, Cedepal CA-210®, and Linseed oil.

Another embodiment comprises cyclosporin A, Cedepal CA-520®, and Linseed oil.

Another embodiment comprises cyclosporin A, Cedepal CA-630®, and Linseed oil.

Another embodiment comprises cyclosporin A, Cedepal CA-720®, and Linseed oil.

Another embodiment comprises cyclosporin A, Cedepal CA-890®, and Linseed oil.

Another embodiment comprises cyclosporin A, Cedepal CA-897®, and Linseed oil.

Another embodiment comprises cyclosporin A, Cedepal CO-210®, and Linseed oil.

Another embodiment comprises cyclosporin A, Cedepal CO-430®, and Linseed oil.

Another embodiment comprises cyclosporin A, Cedepal CO-530®, and Linseed oil.

Another embodiment comprises cyclosporin A, Cedepal CO-630®, and Linseed oil.

Another embodiment comprises cyclosporin A, Cedepal CO-710®, and Linseed oil.

Another embodiment comprises cyclosporin A, Cedepal CO-730®, and Linseed oil.

Another embodiment comprises cyclosporin A, Cedepal CO-880®, and Linseed oil.

Another embodiment comprises cyclosporin A, Cedepal CO-887®, and Linseed oil.

Another embodiment comprises cyclosporin A, Cedepal CO-890®, and Linseed oil.

Another embodiment comprises cyclosporin A, Cedepal CO-897®, and Linseed oil.

Another embodiment comprises cyclosporin A, Cedepal CO-970®, and Linseed oil.

Another embodiment comprises cyclosporin A, Cedepal CO-977®, and Linseed oil.

Another embodiment comprises cyclosporin A, Cedepal CO-990®, and Linseed oil.

Another embodiment comprises cyclosporin A, Cedepal CO-997®, and Linseed oil.

Another embodiment comprises cyclosporin A, Chemal BP 261®, and Linseed oil.

Another embodiment comprises cyclosporin A, Chemal BP 262®, and Linseed oil.

Another embodiment comprises cyclosporin A, Chemal BP 3172®, and Linseed oil.

Another embodiment comprises cyclosporin A, Chemal BP 3174®, and Linseed oil.

Another embodiment comprises cyclosporin A, Chemal DA-4®, and Linseed oil.

Another embodiment comprises cyclosporin A, Chemal DA-6®, and Linseed oil.

Another embodiment comprises cyclosporin A, Chemal DA-9®, and Linseed oil.

Another embodiment comprises cyclosporin A, Chemal LA-23®, and Linseed oil.

Another embodiment comprises cyclosporin A, Chemal LA-4®, and Linseed oil.

Another embodiment comprises cyclosporin A, Chemal LA-9®, and Linseed oil.

Another embodiment comprises cyclosporin A, Chemal OA-20G®, and Linseed oil.

Another embodiment comprises cyclosporin A, Chemal OA-5®, and Linseed oil.

Another embodiment comprises cyclosporin A, Chemal OA-9®, and Linseed oil.

Another embodiment comprises cyclosporin A, Chemal TDA-12®, and Linseed oil.

Another embodiment comprises cyclosporin A, Chemal TDA-15®, and Linseed oil.

Another embodiment comprises cyclosporin A, Chemal TDA-3®, and Linseed oil.

Another embodiment comprises cyclosporin A, Chemal TDA-6®, and Linseed oil.

Another embodiment comprises cyclosporin A, Chemal TDA-9®, and Linseed oil.

Another embodiment comprises cyclosporin A, Chemax CO-16®, and Linseed oil.

Another embodiment comprises cyclosporin A, Chemax CO-200/50®, and Linseed oil.

Another embodiment comprises cyclosporin A, Chemax CO-25®, and Linseed oil.

Another embodiment comprises cyclosporin A, Chemax CO-30®, and Linseed oil.

Another embodiment comprises cyclosporin A, Chemax CO-36®, and Linseed oil.

Another embodiment comprises cyclosporin A, Chemax CO-40®, and Linseed oil.

Another embodiment comprises cyclosporin A, Chemax CO-5®, and Linseed oil.

Another embodiment comprises cyclosporin A, Chemax CO-80®, and Linseed oil.

Another embodiment comprises cyclosporin A, Chemax DNP-150/50®, and Linseed oil.

Another embodiment comprises cyclosporin A, Chemax DNP-18®, and Linseed oil.

Another embodiment comprises cyclosporin A, Chemax DNP-8®, and Linseed oil.

Another embodiment comprises cyclosporin A, Chemax E 200 ML®, and Linseed oil.

Another embodiment comprises cyclosporin A, Chemax E 200 MO®, and Linseed oil.

Another embodiment comprises cyclosporin A, Chemax E 400 ML®, and Linseed oil.

Another embodiment comprises cyclosporin A, Chemax E 400 MO®, and Linseed oil.

Another embodiment comprises cyclosporin A, Chemax E 600 ML®, and Linseed oil.

Another embodiment comprises cyclosporin A, Chemax E 600 MO®, and Linseed oil.

Another embodiment comprises cyclosporin A, Chemax E1000 MO®, and Linseed oil.

Another embodiment comprises cyclosporin A, Chemax E1000 MS®, and Linseed oil.

Another embodiment comprises cyclosporin A, Chemax E200 MS®, and Linseed oil.

Another embodiment comprises cyclosporin A, Chemax E400 MS®, and Linseed oil.

Another embodiment comprises cyclosporin A, Chemax E600 MS®, and Linseed oil.

Another embodiment comprises cyclosporin A, Chemax HCO-16®, and Linseed oil.

Another embodiment comprises cyclosporin A, Chemax HCO-200/50®, and Linseed oil.

Another embodiment comprises cyclosporin A, Chemax HCO-25®, and Linseed oil.

Another embodiment comprises cyclosporin A, Chemax HCO-5®, and Linseed oil.

Another embodiment comprises cyclosporin A, Chemax TO-16®, and Linseed oil.

Another embodiment comprises cyclosporin A, Chemax TO-8®, and Linseed oil.

Another embodiment comprises cyclosporin A, Colalube 3409®, and Linseed oil.

Another embodiment comprises cyclosporin A, Colalube 3411®, and Linseed oil.

Another embodiment comprises cyclosporin A, Colalube 3413®, and Linseed oil.

Another embodiment comprises cyclosporin A, Colalube 3414®, and Linseed oil.

Another embodiment comprises cyclosporin A, Colalube 3415®, and Linseed oil.

Another embodiment comprises cyclosporin A, Colalube 3416®, and Linseed oil.

Another embodiment comprises cyclosporin A, Colalube 3417®, and Linseed oil.

Another embodiment comprises cyclosporin A, Delonic NPE-100®, and Linseed oil.

Another embodiment comprises cyclosporin A, Delonic NPE-30®, and Linseed oil.

Another embodiment comprises cyclosporin A, Delonic NPE-40®, and Linseed oil.

Another embodiment comprises cyclosporin A, Delonic OPE-10®, and Linseed oil.

Another embodiment comprises cyclosporin A, Delonic OPE-12®, and Linseed oil.

Another embodiment comprises cyclosporin A, Delonic OPE-30®, and Linseed oil.

Another embodiment comprises cyclosporin A, Delonic OPE-40®, and Linseed oil.

Another embodiment comprises cyclosporin A, Delonic OPE-5®, and Linseed oil.

Another embodiment comprises cyclosporin A, Delonic OPE-7.5®, and Linseed oil.

Another embodiment comprises cyclosporin A, DePeg 16-CO®, and Linseed oil.

Another embodiment comprises cyclosporin A, DePeg 25-CO®, and Linseed oil.

Another embodiment comprises cyclosporin A, DePeg 30-CO®, and Linseed oil.

Another embodiment comprises cyclosporin A, DePeg 40-CO®, and Linseed oil.

Another embodiment comprises cyclosporin A, DePeg 5-CO®, and Linseed oil.

Another embodiment comprises cyclosporin A, DePeg 80-CO®, and Linseed oil.

Another embodiment comprises cyclosporin A, DeSonic S-100®, and Linseed oil.

Another embodiment comprises cyclosporin A, DeSonic S-114®, and Linseed oil.

Another embodiment comprises cyclosporin A, DeSonic S-405®, and Linseed oil.

Another embodiment comprises cyclosporin A, DeSonic S-45®, and Linseed oil.

Another embodiment comprises cyclosporin A, DeSonic TDA-9®, and Linseed oil.

Another embodiment comprises cyclosporin A, DeThox Acid O-14®, and Linseed oil.

Another embodiment comprises cyclosporin A, DeThox Acid O-16, and Linseed oil.

Another embodiment comprises cyclosporin A, DeThox Acid O-9®, and Linseed oil.

Another embodiment comprises cyclosporin A, DeThox Acid TO-14®, and Linseed oil.

Another embodiment comprises cyclosporin A, DeThox Acid TO-16.53, and Linseed oil.

Another embodiment comprises cyclosporin A, DeThox Acid TO-8.5®, and Linseed oil.

Another embodiment comprises cyclosporin A, Dur-Em 114®, and Linseed oil.

Another embodiment comprises cyclosporin A, Dur-Em 117®, and Linseed oil.

Another embodiment comprises cyclosporin A, Dur-Em 204®, and Linseed oil.

Another embodiment comprises cyclosporin A, Dur-Em 207®, and Linseed oil.

Another embodiment comprises cyclosporin A, Dur-Em 207E®, and Linseed oil.

Another embodiment comprises cyclosporin A, Emerest 2704®, and Linseed oil.

Another embodiment comprises cyclosporin A, Emerest 2712®, and Linseed oil.

Another embodiment comprises cyclosporin A, Emerest 2715®, and Linseed oil.

Another embodiment comprises cyclosporin A, Ethal EH-2®, and Linseed oil.

Another embodiment comprises cyclosporin A, Ethal EH-5®, and Linseed oil.

Another embodiment comprises cyclosporin A, Ethal OA-23®, and Linseed oil.

Another embodiment comprises cyclosporin A, Ethosperse CA-20®, and Linseed oil.

Another embodiment comprises cyclosporin A, Ethosperse LA-12, and Linseed oil.

Another embodiment comprises cyclosporin A, Ethosperse LA-23®, and Linseed oil.

Another embodiment comprises cyclosporin A, Ethosperse LA-4®, and Linseed oil.

Another embodiment comprises cyclosporin A, Ethox 3095®, and Linseed oil.

Another embodiment comprises cyclosporin A, Ethox CO-16®, and Linseed oil.

Another embodiment comprises cyclosporin A, Ethox CO-200®, and Linseed oil.

Another embodiment comprises cyclosporin A, Ethox CO-25®, and Linseed oil.

Another embodiment comprises cyclosporin A, Ethox CO-30®, and Linseed oil.

Another embodiment comprises cyclosporin A, Ethox CO-36®, and Linseed oil.

Another embodiment comprises cyclosporin A, Ethox CO-40®, and Linseed oil.

Another embodiment comprises cyclosporin A, Ethox CO-5®, and Linseed oil.

Another embodiment comprises cyclosporin A, Ethox DO-14®, and Linseed oil.

Another embodiment comprises cyclosporin A, Ethox DO-9®, and Linseed oil.

Another embodiment comprises cyclosporin A, Ethox MA-8®, and Linseed oil.

Another embodiment comprises cyclosporin A, Ethox MI-14®, and Linseed oil.

Another embodiment comprises cyclosporin A, Ethox ML-14®, and Linseed oil.

Another embodiment comprises cyclosporin A, Ethox ML-5®, and Linseed oil.

Another embodiment comprises cyclosporin A, Ethox MO-14®, and Linseed oil.

Another embodiment comprises cyclosporin A, Ethox MO-9®, and Linseed oil.

Another embodiment comprises cyclosporin A, Ethox MS-14®, and Linseed oil.

Another embodiment comprises cyclosporin A, Ethox MS-23®, and Linseed oil.

Another embodiment comprises cyclosporin A, Ethox MS-40®, and Linseed oil.

Another embodiment comprises cyclosporin A, Ethox MS-8®, and Linseed oil.

Another embodiment comprises cyclosporin A, Ethox TO-16®, and Linseed oil.

Another embodiment comprises cyclosporin A, Ethox TO-8®, and Linseed oil.

Another embodiment comprises cyclosporin A, Ethylan SN-120®, and Linseed oil.

Another embodiment comprises cyclosporin A, Ethylan SN-70®, and Linseed oil.

Another embodiment comprises cyclosporin A, Ethylan SN-90®, and Linseed oil.

Another embodiment comprises cyclosporin A, Flexricin 13®, and Linseed oil.

Another embodiment comprises cyclosporin A, Flexricin 15®, and Linseed oil.

Another embodiment comprises cyclosporin A, Genapol 1261®, and Linseed oil.

Another embodiment comprises cyclosporin A, Generol 122 E25®, and Linseed oil.

Another embodiment comprises cyclosporin A, Glicepol 160®, and Linseed oil.

Another embodiment comprises cyclosporin A, Glicepol 560®, and Linseed oil.

Another embodiment comprises cyclosporin A, Glicepol GMS 20®, and Linseed oil.

Another embodiment comprises cyclosporin A, Hetoxide BN-13®, and Linseed oil.

Another embodiment comprises cyclosporin A, Hetoxide DNP-4®, and Linseed oil.

Another embodiment comprises cyclosporin A, Hetoxide DNP-9.6®, and Linseed oil.

Another embodiment comprises cyclosporin A, Hetoxide G-26®, and Linseed oil.

Another embodiment comprises cyclosporin A, Hetoxide G-7®, and Linseed oil.

Another embodiment comprises cyclosporin A, Hetoxide GT-20®, and Linseed oil.

Another embodiment comprises cyclosporin A, Hetoxide HC-40®, and Linseed oil.

Another embodiment comprises cyclosporin A, Hetoxol C-24®, and Linseed oil.

Another embodiment comprises cyclosporin A, Hetoxol CA-10®, and Linseed oil.

Another embodiment comprises cyclosporin A, Hetoxol CA-2®, and Linseed oil.

Another embodiment comprises cyclosporin A, Hetoxol CA-20®, and Linseed oil.

Another embodiment comprises cyclosporin A, Hetoxol CAWS®, and Linseed oil.

Another embodiment comprises cyclosporin A, Hetoxol CD-3®, and Linseed oil.

Another embodiment comprises cyclosporin A, Hetoxol CD-4®, and Linseed oil.

Another embodiment comprises cyclosporin A, Hetoxol CS-15®, and Linseed oil.

Another embodiment comprises cyclosporin A, Hetoxol CS-20®, and Linseed oil.

Another embodiment comprises cyclosporin A, Hetoxol CS-20D®, and Linseed oil.

Another embodiment comprises cyclosporin A, Hetoxol CS-25®, and Linseed oil.

Another embodiment comprises cyclosporin A, Hetoxol CS-50®, and Linseed oil.

Another embodiment comprises cyclosporin A, Hetoxol CS-9®, and Linseed oil.

Another embodiment comprises cyclosporin A, Hetoxol CSA-15®, and Linseed oil.

Another embodiment comprises cyclosporin A, Hetoxol D®, and Linseed oil.

Another embodiment comprises cyclosporin A, Hetoxol G®, and Linseed oil.

Another embodiment comprises cyclosporin A, Hetoxol J®, and Linseed oil.

Another embodiment comprises cyclosporin A, Hetoxol L®, and Linseed oil.

Another embodiment comprises cyclosporin A, Hetoxol L-23®, and Linseed oil.

Another embodiment comprises cyclosporin A, Hetoxol L-4®, and Linseed oil.

Another embodiment comprises cyclosporin A, Hetoxol L-9®, and Linseed oil.

Another embodiment comprises cyclosporin A, Hetoxol LS-9®, and Linseed oil.

Another embodiment comprises cyclosporin A, Hetoxol M-3®, and Linseed oil.

Another embodiment comprises cyclosporin A, Hetoxol OA-10 Special®, and Linseed oil.

Another embodiment comprises cyclosporin A, Hetoxol OA-20 Special®, and Linseed oil.

Another embodiment comprises cyclosporin A, Hetoxol OA-3 Special®, and Linseed oil.

Another embodiment comprises cyclosporin A, Hetoxol OA-5 Special®, and Linseed oil.

Another embodiment comprises cyclosporin A, Hetoxol OL-10®, and Linseed oil.

Another embodiment comprises cyclosporin A, Hetoxol OL-2®, and Linseed oil.

Another embodiment comprises cyclosporin A, Hetoxol OL-23®, and Linseed oil.

Another embodiment comprises cyclosporin A, Hetoxol OL-4®, and Linseed oil.

Another embodiment comprises cyclosporin A, Hetoxol OL-40®, and Linseed oil.

Another embodiment comprises cyclosporin A, Hetoxol OL-5®, and Linseed oil.

Another embodiment comprises cyclosporin A, Hetoxol PLA®, and Linseed oil.

Another embodiment comprises cyclosporin A, Hetoxol SP-15®, and Linseed oil.

Another embodiment comprises cyclosporin A, Hetoxol STA-10®, and Linseed oil.

Another embodiment comprises cyclosporin A, Hetoxol STA-2®, and Linseed oil.

Another embodiment comprises cyclosporin A, Hetoxol STA-30®, and Linseed oil.

Another embodiment comprises cyclosporin A, Hetoxol TD-12®, and Linseed oil.

Another embodiment comprises cyclosporin A, Hetoxol TD-18®, and Linseed oil.

Another embodiment comprises cyclosporin A, Hetoxol TD-3®, and Linseed oil.

Another embodiment comprises cyclosporin A, Hetoxol TD-6®, and Linseed oil.

Another embodiment comprises cyclosporin A, Hetoxol TD-9®, and Linseed oil.

Another embodiment comprises cyclosporin A, Hetoxol TDEP-15®, and Linseed oil.

Another embodiment comprises cyclosporin A, Hetoxol TDEP-63®, and Linseed oil.

Another embodiment comprises cyclosporin A, Iconol 24-12®, and Linseed oil.

Another embodiment comprises cyclosporin A, Iconol 24-23®, and Linseed oil.

Another embodiment comprises cyclosporin A, Iconol 24-4®, and Linseed oil.

Another embodiment comprises cyclosporin A, Iconol DA-4®, and Linseed oil.

Another embodiment comprises cyclosporin A, Iconol DA-6®, and Linseed oil.

Another embodiment comprises cyclosporin A, Iconol TDA-10®, and Linseed oil.

Another embodiment comprises cyclosporin A, Iconol TDA-3®, and Linseed oil.

Another embodiment comprises cyclosporin A, Iconol TDA-6®, and Linseed oil.

Another embodiment comprises cyclosporin A, Iconol TDA-8-90%®, and Linseed oil.

Another embodiment comprises cyclosporin A, Iconol TDA-9®, and Linseed oil.

Another embodiment comprises cyclosporin A, Igepal CA-210®, and Linseed oil.

Another embodiment comprises cyclosporin A, Igepal CA-407®, and Linseed oil.

Another embodiment comprises cyclosporin A, Igepal CA-420®, and Linseed oil.

Another embodiment comprises cyclosporin A, Igepal CA-520®, and Linseed oil.

Another embodiment comprises cyclosporin A, Igepal CA-620®, and Linseed oil.

Another embodiment comprises cyclosporin A, Igepal CA-630®, and Linseed oil.

Another embodiment comprises cyclosporin A, Igepal CA-720®, and Linseed oil.

Another embodiment comprises cyclosporin A, Igepal CA-877®, and Linseed oil.

Another embodiment comprises cyclosporin A, Igepal CA-887®, and Linseed oil.

Another embodiment comprises cyclosporin A, Igepal CA-897®, and Linseed oil.

Another embodiment comprises cyclosporin A, Igepal CO-210®, and Linseed oil.

Another embodiment comprises cyclosporin A, Igepal CO-430®, and Linseed oil.

Another embodiment comprises cyclosporin A, Igepal CO-520®, and Linseed oil.

Another embodiment comprises cyclosporin A, Igepal CO-530®, and Linseed oil.

Another embodiment comprises cyclosporin A, Igepal CO-610®, and Linseed oil.

Another embodiment comprises cyclosporin A, Igepal CO-630®, and Linseed oil.

Another embodiment comprises cyclosporin A, Igepal CO-630 Special®, and Linseed oil.

Another embodiment comprises cyclosporin A, Igepal CO-660®, and Linseed oil.

Another embodiment comprises cyclosporin A, Igepal CO-710®, and Linseed oil.

Another embodiment comprises cyclosporin A, Igepal CO-720®, and Linseed oil.

Another embodiment comprises cyclosporin A, Igepal CO-730®, and Linseed oil.

Another embodiment comprises cyclosporin A, Igepal CO-738®, and Linseed oil.

Another embodiment comprises cyclosporin A, Igepal CO-850®, and Linseed oil.

Another embodiment comprises cyclosporin A, Igepal CO-880®, and Linseed oil.

Another embodiment comprises cyclosporin A, Igepal CO-887®, and Linseed oil.

Another embodiment comprises cyclosporin A, Igepal CO-890®, and Linseed oil.

Another embodiment comprises cyclosporin A, Igepal CO-897®, and Linseed oil.

Another embodiment comprises cyclosporin A, Igepal CO-970®, and Linseed oil.

Another embodiment comprises cyclosporin A, Igepal CO-977®, and Linseed oil.

Another embodiment comprises cyclosporin A, Igepal CO-987®, and Linseed oil.

Another embodiment comprises cyclosporin A, Igepal CO-990 FLK®, and Linseed oil.

Another embodiment comprises cyclosporin A, Igepal CO-997®, and Linseed oil.

Another embodiment comprises cyclosporin A, Igepal DM-430®, and Linseed oil.

Another embodiment comprises cyclosporin A, Igepal DM-530®, and Linseed oil.

Another embodiment comprises cyclosporin A, Igepal DM-710®, and Linseed oil.

Another embodiment comprises cyclosporin A, Igepal DM-730®, and Linseed oil.

Another embodiment comprises cyclosporin A, Igepal DM-880®, and Linseed oil.

Another embodiment comprises cyclosporin A, Igepal DM-970 FLK®, and Linseed oil.

Another embodiment comprises cyclosporin A, Igepal OD-410®, and Linseed oil.

Another embodiment comprises cyclosporin A, Igepal RC-520®, and Linseed oil.

Another embodiment comprises cyclosporin A, Igepal RC-620®, and Linseed oil.

Another embodiment comprises cyclosporin A, Igepal RC-630®, and Linseed oil.

Another embodiment comprises cyclosporin A, Lexemul CS-20®, and Linseed oil.

Another embodiment comprises cyclosporin A, Lipo GMS 450®, and Linseed oil.

Another embodiment comprises cyclosporin A, Lipo Polyglycol 1000®, and Linseed oil.

Another embodiment comprises cyclosporin A, Lipo Polyglycol 200®, and Linseed oil.

Another embodiment comprises cyclosporin A, Lipo Polyglycol 300®, and Linseed oil.

Another embodiment comprises cyclosporin A, Lipo Polyglycol 3350®, and Linseed oil.

Another embodiment comprises cyclosporin A, Lipo Polyglycol 400®, and Linseed oil.

Another embodiment comprises cyclosporin A, Lipo Polyglycol 600®, and Linseed oil.

Another embodiment comprises cyclosporin A, Lipocol C-10®, and Linseed oil.

Another embodiment comprises cyclosporin A, Lipocol C-2®, and Linseed oil.

Another embodiment comprises cyclosporin A, Lipocol C-20®, and Linseed oil.

Another embodiment comprises cyclosporin A, Lipocol L-12®, and Linseed oil.

Another embodiment comprises cyclosporin A, Lipocol L-23®, and Linseed oil.

Another embodiment comprises cyclosporin A, Lipocol L-4®, and Linseed oil.

Another embodiment comprises cyclosporin A, Lipocol O-10®, and Linseed oil.

Another embodiment comprises cyclosporin A, Lipocol O-2®, and Linseed oil.

Another embodiment comprises cyclosporin A, Lipocol O-20®, and Linseed oil.

Another embodiment comprises cyclosporin A, Lipocol O-5®, and Linseed oil.

Another embodiment comprises cyclosporin A, Lipocol S-10®, and Linseed oil.

Another embodiment comprises cyclosporin A, Lipocol S-2®, and Linseed oil.

Another embodiment comprises cyclosporin A, Lipocol S-20®, and Linseed oil.

Another embodiment comprises cyclosporin A, Lipocol SC-15®, and Linseed oil.

Another embodiment comprises cyclosporin A, Lipocol SC-20®, and Linseed oil.

Another embodiment comprises cyclosporin A, Lipocol SC-4®, and Linseed oil.

Another embodiment comprises cyclosporin A, Lipocol TD-12®, and Linseed oil.

Another embodiment comprises cyclosporin A, Lumulse 40-O K®, and Linseed oil.

Another embodiment comprises cyclosporin A, Lumulse 42-O K®, and Linseed oil.

Another embodiment comprises cyclosporin A, Lumulse GMT-40®, and Linseed oil.

Another embodiment comprises cyclosporin A, Lumulse PEG 1450®, and Linseed oil.

Another embodiment comprises cyclosporin A, Lumulse PEG 1450 NF®, and Linseed oil.

Another embodiment comprises cyclosporin A, Lumulse PEG 8000®, and Linseed oil.

Another embodiment comprises cyclosporin A, Lumulse PGO®, and Linseed oil.

Another embodiment comprises cyclosporin A, Lumulse POE (2) Oleyl Amine®, and Linseed oil.

Another embodiment comprises cyclosporin A, Magrabar PGO-104®, and Linseed oil.

Another embodiment comprises cyclosporin A, Mapeg 200 ML®, and Linseed oil.

Another embodiment comprises cyclosporin A, Mapeg 400 MO®, and Linseed oil.

Another embodiment comprises cyclosporin A, Mapeg 400 MOT®, and Linseed oil.

Another embodiment comprises cyclosporin A, Mapeg 400DO®, and Linseed oil.

Another embodiment comprises cyclosporin A, Mapeg 400DOT®, and Linseed oil.

Another embodiment comprises cyclosporin A, Mapeg 600DOT®, and Linseed oil.

Another embodiment comprises cyclosporin A, Marlox B 24/80®, and Linseed oil.

Another embodiment comprises cyclosporin A, Marlox FK 86®, and Linseed oil.

Another embodiment comprises cyclosporin A, Marlox MO 154®, and Linseed oil.

Another embodiment comprises cyclosporin A, Mazox CDA CG®, and Linseed oil.

Another embodiment comprises cyclosporin A, Monoglycerides Glyceryl Monestearate®, and Linseed oil.

Another embodiment comprises cyclosporin A, Myrj 45®, and Linseed oil.

Another embodiment comprises cyclosporin A, Myrj 52®, and Linseed oil.

Another embodiment comprises cyclosporin A, Myrj 52S®, and Linseed oil.

Another embodiment comprises cyclosporin A, Myrj 53®, and Linseed oil.

Another embodiment comprises cyclosporin A, Myrj 59®, and Linseed oil.

Another embodiment comprises cyclosporin A, Norfox NP-12®, and Linseed oil.

Another embodiment comprises cyclosporin A, Norfox NP-4®, and Linseed oil.

Another embodiment comprises cyclosporin A, Norfox NP-6®, and Linseed oil.

Another embodiment comprises cyclosporin A, Norfox NP-887®, and Linseed oil.

Another embodiment comprises cyclosporin A, Norfox NP-9®, and Linseed oil.

Another embodiment comprises cyclosporin A, Norfox NP-977®, and Linseed oil.

Another embodiment comprises cyclosporin A, Norfox OP-100®, and Linseed oil.

Another embodiment comprises cyclosporin A, Norfox OP-102®, and Linseed oil.

Another embodiment comprises cyclosporin A, Norfox OP-114®, and Linseed oil.

Another embodiment comprises cyclosporin A, Norfox OP-45®, and Linseed oil.

Another embodiment comprises cyclosporin A, Pegosperse 100-L®, and Linseed oil.

Another embodiment comprises cyclosporin A, Pegosperse 100-O®, and Linseed oil.

Another embodiment comprises cyclosporin A, Pegosperse 100-S®, and Linseed oil.

Another embodiment comprises cyclosporin A, Pegosperse 1500-MS®, and Linseed oil.

Another embodiment comprises cyclosporin A, Pegosperse 1750-MS®, and Linseed oil.

Another embodiment comprises cyclosporin A, Pegosperse 200 DL®, and Linseed oil.

Another embodiment comprises cyclosporin A, Pegosperse 200-ML®, and Linseed oil.

Another embodiment comprises cyclosporin A, Pegosperse 400-DL®, and Linseed oil.

Another embodiment comprises cyclosporin A, Pegosperse 400-DO®, and Linseed oil.

Another embodiment comprises cyclosporin A, Pegosperse 400-DS®, and Linseed oil.

Another embodiment comprises cyclosporin A, Pegosperse 400-ML®, and Linseed oil.

Another embodiment comprises cyclosporin A, Pegosperse 400-MO®, and Linseed oil.

Another embodiment comprises cyclosporin A, Pegosperse 400-MOT®, and Linseed oil.

Another embodiment comprises cyclosporin A, Pegosperse 400-MS®, and Linseed oil.

Another embodiment comprises cyclosporin A, Pegosperse 50-DS®, and Linseed oil.

Another embodiment comprises cyclosporin A, Pegosperse 50-MS®, and Linseed oil.

Another embodiment comprises cyclosporin A, Pegosperse 600-ML®, and Linseed oil.

Another embodiment comprises cyclosporin A, Pegosperse 600-MS®, and Linseed oil.

Another embodiment comprises cyclosporin A, Plurafac A-38®, and Linseed oil.

Another embodiment comprises cyclosporin A, Plurafac B-25-5®, and Linseed oil.

Another embodiment comprises cyclosporin A, Plurafac B-26®, and Linseed oil.

Another embodiment comprises cyclosporin A, Plurafac D-25®, and Linseed oil.

Another embodiment comprises cyclosporin A, Plurafac LF 131®, and Linseed oil.

Another embodiment comprises cyclosporin A, Plurafac LF 4030®, and Linseed oil.

Another embodiment comprises cyclosporin A, Plurafac LF 7000®, and Linseed oil.

Another embodiment comprises cyclosporin A, Plurafac RA-20®, and Linseed oil.

Another embodiment comprises cyclosporin A, Plurafac RA-30®, and Linseed oil.

Another embodiment comprises cyclosporin A, Plurafac RA-40®, and Linseed oil.

Another embodiment comprises cyclosporin A, Pluronic 10R5®, and Linseed oil.

Another embodiment comprises cyclosporin A, Pluronic 17R2®, and Linseed oil.

Another embodiment comprises cyclosporin A, Pluronic 17R4®, and Linseed oil.

Another embodiment comprises cyclosporin A, Pluronic 25R2®, and Linseed oil.

Another embodiment comprises cyclosporin A, Pluronic 25R4®, and Linseed oil.

Another embodiment comprises cyclosporin A, Pluronic 31R1®, and Linseed oil.

Another embodiment comprises cyclosporin A, Pluronic F108®, and Linseed oil.

Another embodiment comprises cyclosporin A, Pluronic F127®, and Linseed oil.

Another embodiment comprises cyclosporin A, Pluronic F38®, and Linseed oil.

Another embodiment comprises cyclosporin A, Pluronic F68®, and Linseed oil.

Another embodiment comprises cyclosporin A, Pluronic F68LF®, and Linseed oil.

Another embodiment comprises cyclosporin A, Pluronic F77®, and Linseed oil.

Another embodiment comprises cyclosporin A, Pluronic F87®, and Linseed oil.

Another embodiment comprises cyclosporin A, Pluronic F88®, and Linseed oil.

Another embodiment comprises cyclosporin A, Pluronic F98®, and Linseed oil.

Another embodiment comprises cyclosporin A, Pluronic L10®, and Linseed oil.

Another embodiment comprises cyclosporin A, Pluronic L101®, and Linseed oil.

Another embodiment comprises cyclosporin A, Pluronic L121®, and Linseed oil.

Another embodiment comprises cyclosporin A, Pluronic L31®, and Linseed oil.

Another embodiment comprises cyclosporin A, Pluronic L35®, and Linseed oil.

Another embodiment comprises cyclosporin A, Pluronic L43®, and Linseed oil.

Another embodiment comprises cyclosporin A, Pluronic L44®, and Linseed oil.

Another embodiment comprises cyclosporin A, Pluronic L61®, and Linseed oil.

Another embodiment comprises cyclosporin A, Pluronic L62®, and Linseed oil.

Another embodiment comprises cyclosporin A, Pluronic L62D®, and Linseed oil.

Another embodiment comprises cyclosporin A, Pluronic L62LF®, and Linseed oil.

Another embodiment comprises cyclosporin A, Pluronic L64®, and Linseed oil.

Another embodiment comprises cyclosporin A, Pluronic L81®, and Linseed oil.

Another embodiment comprises cyclosporin A, Pluronic L92®, and Linseed oil.

Another embodiment comprises cyclosporin A, Pluronic P103®, and Linseed oil.

Another embodiment comprises cyclosporin A, Pluronic P104®, and Linseed oil.

Another embodiment comprises cyclosporin A, Pluronic P105®, and Linseed oil.

Another embodiment comprises cyclosporin A, Pluronic P123®, and Linseed oil.

Another embodiment comprises cyclosporin A, Pluronic P65®, and Linseed oil.

Another embodiment comprises cyclosporin A, Pluronic P84®, and Linseed oil.

Another embodiment comprises cyclosporin A, Pluronic P85®, and Linseed oil.

Another embodiment comprises cyclosporin A, Polyaldo 10-2-P®, and Linseed oil.

Another embodiment comprises cyclosporin A, Polyaldo DGDO®, and Linseed oil.

Another embodiment comprises cyclosporin A, Polyaldo HGDS®, and Linseed oil.

Another embodiment comprises cyclosporin A, Polyaldo TGMS®, and Linseed oil.

Another embodiment comprises cyclosporin A, Protegin V®, and Linseed oil.

Another embodiment comprises cyclosporin A, Protegin W®, and Linseed oil.

Another embodiment comprises cyclosporin A, Protegin WX®, and Linseed oil.

Another embodiment comprises cyclosporin A, Protegin XV®, and Linseed oil.

Another embodiment comprises cyclosporin A, Rhodasurf BC-420®, and Linseed oil.

Another embodiment comprises cyclosporin A, Rhodasurf BC-610®, and Linseed oil.

Another embodiment comprises cyclosporin A, Rhodasurf BC-630®, and Linseed oil.

Another embodiment comprises cyclosporin A, Rhodasurf BC-720®, and Linseed oil.

Another embodiment comprises cyclosporin A, Rhodasurf BC-840®, and Linseed oil.

Another embodiment comprises cyclosporin A, Rhodasurf L 1®, and Linseed oil.

Another embodiment comprises cyclosporin A, Rhodasurf L 12®, and Linseed oil.

Another embodiment comprises cyclosporin A, Rhodasurf L 2®, and Linseed oil.

Another embodiment comprises cyclosporin A, Rhodasur L 20®, and Linseed oil.

Another embodiment comprises cyclosporin A, Rhodasurf L 3®, and Linseed oil.

Another embodiment comprises cyclosporin A, Rhodasur L 30®, and Linseed oil.

Another embodiment comprises cyclosporin A, Rhodasurf L 4®, and Linseed oil.

Another embodiment comprises cyclosporin A, Rhodasur L 50®, and Linseed oil.

Another embodiment comprises cyclosporin A, Rhodasurf L 7/90®, and Linseed oil.

Another embodiment comprises cyclosporin A, Rhodasurf LA-3®, and Linseed oil.

Another embodiment comprises cyclosporin A, Rhodasurf LA-7®, and Linseed oil.

Another embodiment comprises cyclosporin A, Rhodasurf LAN-23®, and Linseed oil.

Another embodiment comprises cyclosporin A, Rhodasurf ON-870®, and Linseed oil.

Another embodiment comprises cyclosporin A, Rhodasurf ON-877®, and Linseed oil.

Another embodiment comprises cyclosporin A, Rhodasurf TDA-8/5®, and Linseed oil.

Another embodiment comprises cyclosporin A, Ritapro 100®, and Linseed oil.

Another embodiment comprises cyclosporin A, Ritapro 165®, and Linseed oil.

Another embodiment comprises cyclosporin A, Ritapro 200®, and Linseed oil.

Another embodiment comprises cyclosporin A, Ritapro 300®, and Linseed oil.

Another embodiment comprises cyclosporin A, Ritoleth 10®, and Linseed oil.

Another embodiment comprises cyclosporin A, Ritoleth 2®, and Linseed oil.

Another embodiment comprises cyclosporin A, Ritoleth 20®, and Linseed oil.

Another embodiment comprises cyclosporin A, Ritoleth 5®, and Linseed oil.

Another embodiment comprises cyclosporin A, Santone 10-10-0®, and Linseed oil.

Another embodiment comprises cyclosporin A, Santone 3-1-S XTR®, and Linseed oil.

Another embodiment comprises cyclosporin A, Santone 3-1-SH®, and Linseed oil.

Another embodiment comprises cyclosporin A, Santone 8-1-O®, and Linseed oil.

Another embodiment comprises cyclosporin A, Solulan 16®, and Linseed oil.

Another embodiment comprises cyclosporin A, Solulan 75®, and Linseed oil.

Another embodiment comprises cyclosporin A, Solulan C-24®, and Linseed oil.

Another embodiment comprises cyclosporin A, Solulan L-575®, and Linseed oil.

Another embodiment comprises cyclosporin A, Standamox CAW®, and Linseed oil.

Another embodiment comprises cyclosporin A, Surfine AZI-A®, and Linseed oil.

Another embodiment comprises cyclosporin A, Surfonic CO-15®, and Linseed oil.

Another embodiment comprises cyclosporin A, Surfonic CO-25®, and Linseed oil.

Another embodiment comprises cyclosporin A, Surfonic CO-30®, and Linseed oil.

Another embodiment comprises cyclosporin A, Surfonic CO-36®, and Linseed oil.

Another embodiment comprises cyclosporin A, Surfonic CO-42®, and Linseed oil.

Another embodiment comprises cyclosporin A, Surfonic E400-MO®, and Linseed oil.

Another embodiment comprises cyclosporin A, Surfonic L12-3®, and Linseed oil.

Another embodiment comprises cyclosporin A, Surfonic L12-6®, and Linseed oil.

Another embodiment comprises cyclosporin A, Surfonic L12-8®, and Linseed oil.

Another embodiment comprises cyclosporin A, Surfonic L24-12®, and Linseed oil.

Another embodiment comprises cyclosporin A, Surfonic L24-2®, and Linseed oil.

Another embodiment comprises cyclosporin A, Surfonic L24-22®, and Linseed oil.

Another embodiment comprises cyclosporin A, Surfonic L24-3®, and Linseed oil.

Another embodiment comprises cyclosporin A, Surfonic L24-4®, and Linseed oil.

Another embodiment comprises cyclosporin A, Surfonic L24-5®, and Linseed oil.

Another embodiment comprises cyclosporin A, Surfonic L24-7®, and Linseed oil.

Another embodiment comprises cyclosporin A, Surfonic L24-7.1®, and Linseed oil.

Another embodiment comprises cyclosporin A, Surfonic L24-9®, and Linseed oil.

Another embodiment comprises cyclosporin A, Surfonic LF-17®, and Linseed oil.

Another embodiment comprises cyclosporin A, Surfonic LF-18®, and Linseed oil.

Another embodiment comprises cyclosporin A, Surfonic LF-37®, and Linseed oil.

Another embodiment comprises cyclosporin A, Surfonic LF-41®, and Linseed oil.

Another embodiment comprises cyclosporin A, Surfonic LF-42®, and Linseed oil.

Another embodiment comprises cyclosporin A, Surfonic LF-47®, and Linseed oil.

Another embodiment comprises cyclosporin A, Surfonic LF-68®, and Linseed oil.

Another embodiment comprises cyclosporin A, Surfonic N-10®, and Linseed oil.

Another embodiment comprises cyclosporin A, Surfonic N-100®, and Linseed oil.

Another embodiment comprises cyclosporin A, Surfonic N-102®, and Linseed oil.

Another embodiment comprises cyclosporin A, Surfonic N-120®, and Linseed oil.

Another embodiment comprises cyclosporin A, Surfonic N-150®, and Linseed oil.

Another embodiment comprises cyclosporin A, Surfonic N-200®, and Linseed oil.

Another embodiment comprises cyclosporin A, Surfonic N-300®, and Linseed oil.

Another embodiment comprises cyclosporin A, Surfonic N-31.5®, and Linseed oil.

Another embodiment comprises cyclosporin A, Surfonic N-40®, and Linseed oil.

Another embodiment comprises cyclosporin A, Surfonic N-400®, and Linseed oil.

Another embodiment comprises cyclosporin A, Surfonic N-60®, and Linseed oil.

Another embodiment comprises cyclosporin A, Surfonic N-85®, and Linseed oil.

Another embodiment comprises cyclosporin A, Surfonic N-95®, and Linseed oil.

Another embodiment comprises cyclosporin A, Sunonic NB-18®, and Linseed oil.

Another embodiment comprises cyclosporin A, Surfonic NB-23®, and Linseed oil.

Another embodiment comprises cyclosporin A, Sunonic NB-25®, and Linseed oil.

Another embodiment comprises cyclosporin A, Sunonic NB-307®, and Linseed oil.

Another embodiment comprises cyclosporin A, Sunonic NB-31®, and Linseed oil.

Another embodiment comprises cyclosporin A, Surfonic NB-41®, and Linseed oil.

Another embodiment comprises cyclosporin A, Surfonic NB-43®, and Linseed oil.

Another embodiment comprises cyclosporin A, Surfonic OP-100®, and Linseed oil.

Another embodiment comprises cyclosporin A, Surfonic OP-120®, and Linseed oil.

Another embodiment comprises cyclosporin A, Surfonic OP-15®, and Linseed oil.

Another embodiment comprises cyclosporin A, Surfonic OP-35®, and Linseed oil.

Another embodiment comprises cyclosporin A, Surfonic OP-70®, and Linseed oil.

Another embodiment comprises cyclosporin A, Surfonic P1®, and Linseed oil.

Another embodiment comprises cyclosporin A, Surfonic P3®, and Linseed oil.

Another embodiment comprises cyclosporin A, Surfonic P5®, and Linseed oil.

Another embodiment comprises cyclosporin A, Surfonic P6®, and Linseed oil.

Another embodiment comprises cyclosporin A, Surfonic POA-17R2®, and Linseed oil.

Another embodiment comprises cyclosporin A, Surfonic POA-25R2®, and Linseed oil.

Another embodiment comprises cyclosporin A, Surfonic TDA-11®, and Linseed oil.

Another embodiment comprises cyclosporin A, Surfonic TDA-3B®, and Linseed oil.

Another embodiment comprises cyclosporin A, Surfonic TDA-6®, and Linseed oil.

Another embodiment comprises cyclosporin A, Surfonic TDA-6/88®, and Linseed oil.

Another embodiment comprises cyclosporin A, Surfonic TDA-8®, and Linseed oil.

Another embodiment comprises cyclosporin A, Surfonic TDA-8/90®, and Linseed oil.

Another embodiment comprises cyclosporin A, Surfonic TDA-9®, and Linseed oil.

Another embodiment comprises cyclosporin A, Surfonic TX-CCN®, and Linseed oil.

Another embodiment comprises cyclosporin A, Tagat CH 40®, and Linseed oil.

Another embodiment comprises cyclosporin A, Tagat CH 60®, and Linseed oil.

Another embodiment comprises cyclosporin A, Tagat L 2®, and Linseed oil.

Another embodiment comprises cyclosporin A, Tagat S®, and Linseed oil.

Another embodiment comprises cyclosporin A, Tagat S 2®, and Linseed oil.

Another embodiment comprises cyclosporin A, Tally 100 Plus®, and Linseed oil.

Another embodiment comprises cyclosporin A, T-Det A106®, and Linseed oil.

Another embodiment comprises cyclosporin A, T-Det A109®, and Linseed oil.

Another embodiment comprises cyclosporin A, T-Det A136®, and Linseed oil.

Another embodiment comprises cyclosporin A, T-Det A139®, and Linseed oil.

Another embodiment comprises cyclosporin A, T-Det A2412®, and Linseed oil.

Another embodiment comprises cyclosporin A, T-Det A243®, and Linseed oil.

Another embodiment comprises cyclosporin A, T-Det A247®, and Linseed oil.

Another embodiment comprises cyclosporin A, T-Det A249®, and Linseed oil.

Another embodiment comprises cyclosporin A, T-Det A467®, and Linseed oil.

Another embodiment comprises cyclosporin A, T-Det COE®, and Linseed oil.

Another embodiment comprises cyclosporin A, T-Det DD-10®, and Linseed oil.

Another embodiment comprises cyclosporin A, T-Det DD-5®, and Linseed oil.

Another embodiment comprises cyclosporin A, T-Det DD-7®, and Linseed oil.

Another embodiment comprises cyclosporin A, T-Det EPO-104®, and Linseed oil.

Another embodiment comprises cyclosporin A, T-Det EPO-61®, and Linseed oil.

Another embodiment comprises cyclosporin A, T-Det EPO-62®, and Linseed oil.

Another embodiment comprises cyclosporin A, T-Det EPO-64®, and Linseed oil.

Another embodiment comprises cyclosporin A, T-Det N-1.5®, and Linseed oil.

Another embodiment comprises cyclosporin A, T-Det N-10.5®, and Linseed oil.

Another embodiment comprises cyclosporin A, T-Det N-100®, and Linseed oil.

Another embodiment comprises cyclosporin A, T-Det N-1007®, and Linseed oil.

Another embodiment comprises cyclosporin A, T-Det N-12®, and Linseed oil.

Another embodiment comprises cyclosporin A, T-Det N-14®, and Linseed oil.

Another embodiment comprises cyclosporin A, T-Det N-20®, and Linseed oil.

Another embodiment comprises cyclosporin A, T-Det N-30®, and Linseed oil.

Another embodiment comprises cyclosporin A, T-Det N-307®, and Linseed oil.

Another embodiment comprises cyclosporin A, T-Det N-4®, and Linseed oil.

Another embodiment comprises cyclosporin A, T-Det N-40®, and Linseed oil.

Another embodiment comprises cyclosporin A, T-Det N-407®, and Linseed oil.

Another embodiment comprises cyclosporin A, T-Det N-50®, and Linseed oil.

Another embodiment comprises cyclosporin A, T-Det N-507®, and Linseed oil.

Another embodiment comprises cyclosporin A, T-Det N-6®, and Linseed oil.

Another embodiment comprises cyclosporin A, T-Det N-70®, and Linseed oil.

Another embodiment comprises cyclosporin A, T-Det N-8®, and Linseed oil.

Another embodiment comprises cyclosporin A, T-Det N-9.5®, and Linseed oil.

Another embodiment comprises cyclosporin A, T-Det O-12®, and Linseed oil.

Another embodiment comprises cyclosporin A, T-Det O-165®, and Linseed oil.

Another embodiment comprises cyclosporin A, T-Det O-307®, and Linseed oil.

Another embodiment comprises cyclosporin A, T-Det O-40®, and Linseed oil.

Another embodiment comprises cyclosporin A, T-Det O-407®, and Linseed oil.

Another embodiment comprises cyclosporin A, T-Det O-6®, and Linseed oil.

Another embodiment comprises cyclosporin A, T-Det O-8®, and Linseed oil.

Another embodiment comprises cyclosporin A, T-Det O-9®, and Linseed oil.

Another embodiment comprises cyclosporin A, Tegin®, and Linseed oil.

Another embodiment comprises cyclosporin A, Tergitol 15-S-12®, and Linseed oil.

Another embodiment comprises cyclosporin A, Tergitol 15-S-15®, and Linseed oil.

Another embodiment comprises cyclosporin A, Tergitol 15-S-20®, and Linseed oil.

Another embodiment comprises cyclosporin A, Tergitol 15-S-20 (80AQ)®, and Linseed oil.

Another embodiment comprises cyclosporin A, Tergitol 15-S-3®, and Linseed oil.

Another embodiment comprises cyclosporin A, Tergitol 15-S-30®, and Linseed oil.

Another embodiment comprises cyclosporin A, Tergitol 15-S-40®, and Linseed oil.

Another embodiment comprises cyclosporin A, Tergitol 15-S-40 (70AQ)®, and Linseed oil.

Another embodiment comprises cyclosporin A, Tergitol 15-S-5®, and Linseed oil.

Another embodiment comprises cyclosporin A, Tergitol 15-S-7®, and Linseed oil.

Another embodiment comprises cyclosporin A, Tergitol 15-S-9®, and Linseed oil.

Another embodiment comprises cyclosporin A, Tergitol L-101®, and Linseed oil.

Another embodiment comprises cyclosporin A, Tergitol L-61®, and Linseed oil.

Another embodiment comprises cyclosporin A, Tergitol L-62®, and Linseed oil.

Another embodiment comprises cyclosporin A, Tergitol L-64®, and Linseed oil.

Another embodiment comprises cyclosporin A, Tergitol L-81®, and Linseed oil.

Another embodiment comprises cyclosporin A, Tergitol NP-10®, and Linseed oil.

Another embodiment comprises cyclosporin A, Tergitol NP-11®, and Linseed oil.

Another embodiment comprises cyclosporin A, Tergitol NP-12®, and Linseed oil.

Another embodiment comprises cyclosporin A, Tergitol NP-13®, and Linseed oil.

Another embodiment comprises cyclosporin A, Tergitol NP-15®, and Linseed oil.

Another embodiment comprises cyclosporin A, Tergitol NP-30®, and Linseed oil.

Another embodiment comprises cyclosporin A, Tergitol NP-30 (70 AQ)®, and Linseed oil.

Another embodiment comprises cyclosporin A, Tergitol NP-4®, and Linseed oil.

Another embodiment comprises cyclosporin A, Tergitol NP-40®, and Linseed oil.

Another embodiment comprises cyclosporin A, Tergitol NP-40 (70 AQ)®, and Linseed oil.

Another embodiment comprises cyclosporin A, Tergitol NP-50 (70 AQ)®, and Linseed oil.

Another embodiment comprises cyclosporin A, Tergitol NP-55 (70 AQ)®, and Linseed oil.

Another embodiment comprises cyclosporin A, Tergitol NP-6®, and Linseed oil.

Another embodiment comprises cyclosporin A, Tergitol NP-7®, and Linseed oil.

Another embodiment comprises cyclosporin A, Tergitol NP-70 (70 AQ)®, and Linseed oil.

Another embodiment comprises cyclosporin A, Tergitol NP-8®, and Linseed oil.

Another embodiment comprises cyclosporin A, Tergitol NP-9®, and Linseed oil.

Another embodiment comprises cyclosporin A, Tergitol NP-9.5®, and Linseed oil.

Another embodiment comprises cyclosporin A, Tergitol TMN-10®, and Linseed oil.

Another embodiment comprises cyclosporin A, Tergitol TMN-3®, and Linseed oil.

Another embodiment comprises cyclosporin A, Tergitol TMN-6®, and Linseed oil.

Another embodiment comprises cyclosporin A, Tergitol XD®, and Linseed oil.

Another embodiment comprises cyclosporin A, Tergitol XH®, and Linseed oil.

Another embodiment comprises cyclosporin A, Tergitol XJ®, and Linseed oil.

Another embodiment comprises cyclosporin A, Tetronic 1107®, and Linseed oil.

Another embodiment comprises cyclosporin A, Tetronic 1307®, and Linseed oil.

Another embodiment comprises cyclosporin A, Tetronic 150R1®, and Linseed oil.

Another embodiment comprises cyclosporin A, Tetronic 304®, and Linseed oil.

Another embodiment comprises cyclosporin A, Tetronic 701®, and Linseed oil.

Another embodiment comprises cyclosporin A, Tetronic 901®, and Linseed oil.

Another embodiment comprises cyclosporin A, Tetronic 904®, and Linseed oil.

Another embodiment comprises cyclosporin A, Tetronic 908®, and Linseed oil.

Another embodiment comprises cyclosporin A, Tetronic 90R4®, and Linseed oil.

Another embodiment comprises cyclosporin A, Tomadol 1-3®, and Linseed oil.

Another embodiment comprises cyclosporin A, Tomadol 1-5®, and Linseed oil.

Another embodiment comprises cyclosporin A, Tomadol 1-7®, and Linseed oil.

Another embodiment comprises cyclosporin A, Tomadol 23-1®, and Linseed oil.

Another embodiment comprises cyclosporin A, Tomadol 23-3®, and Linseed oil.

Another embodiment comprises cyclosporin A, Tomadol 23-5®, and Linseed oil.

Another embodiment comprises cyclosporin A, Tomadol 23-6.5®, and Linseed oil.

Another embodiment comprises cyclosporin A, Tomadol 25-12®, and Linseed oil.

Another embodiment comprises cyclosporin A, Tomadol 25-3®, and Linseed oil.

Another embodiment comprises cyclosporin A, Tomadol 25-7®, and Linseed oil.

Another embodiment comprises cyclosporin A, Tomadol 25-9®, and Linseed oil.

Another embodiment comprises cyclosporin A, Tomadol 45-13®, and Linseed oil.

Another embodiment comprises cyclosporin A, Tomadol 45-2.5®, and Linseed oil.

Another embodiment comprises cyclosporin A, Tomadol 45-7®, and Linseed oil.

Another embodiment comprises cyclosporin A, Tomadol 91-2.5®, and Linseed oil.

Another embodiment comprises cyclosporin A, Tomadol 91-6®, and Linseed oil.

Another embodiment comprises cyclosporin A, Tomadol 91-8®, and Linseed oil.

Another embodiment comprises cyclosporin A, Triton X-100®, and Linseed oil.

Another embodiment comprises cyclosporin A, Triton X-102®, and Linseed oil.

Another embodiment comprises cyclosporin A, Triton X-114®, and Linseed oil.

Another embodiment comprises cyclosporin A, Triton X-15®, and Linseed oil.

Another embodiment comprises cyclosporin A, Triton X-165 (70%)®, and Linseed oil.

Another embodiment comprises cyclosporin A, Triton X-207®, and Linseed oil.

Another embodiment comprises cyclosporin A, Triton X-305 (70%)®, and Linseed oil.

Another embodiment comprises cyclosporin A, Triton X-35®, and Linseed oil.

Another embodiment comprises cyclosporin A, Triton X-405 (70%)®, and Linseed oil.

Another embodiment comprises cyclosporin A, Triton X-45®, and Linseed oil.

Another embodiment comprises cyclosporin A, Triton X-705 (70%)®, and Linseed oil.

Another embodiment comprises cyclosporin A, Volpo S-10®, and Linseed oil.

Another embodiment comprises cyclosporin A, Volpo S-2®, and Linseed oil.

Another embodiment comprises cyclosporin A, Volpo S-20®, and Linseed oil.

Another embodiment comprises cyclosporin A, Volpo-10®, and Linseed oil.

Another embodiment comprises cyclosporin A, Volpo-20®, and Linseed oil.

Another embodiment comprises cyclosporin A, Volpo-3®, and Linseed oil.

Another embodiment comprises cyclosporin A, Volpo-5®, and Linseed oil.

Another embodiment comprises cyclosporin A, Abex 2515®, and Rapeseed oil.

Another embodiment comprises cyclosporin A, Abex 2525/400®, and Rapeseed oil.

Another embodiment comprises cyclosporin A, Abex 2535®, and Rapeseed oil.

Another embodiment comprises cyclosporin A, Abex 2545®, and Rapeseed oil.

Another embodiment comprises cyclosporin A, Acconon E®, and Rapeseed oil.

Another embodiment comprises cyclosporin A, Alfonic 1216CO-9®, and Rapeseed oil.

Another embodiment comprises cyclosporin A, Alfonic 1412-3®, and Rapeseed oil.

Another embodiment comprises cyclosporin A, Alfonic TDA-12®, and Rapeseed oil.

Another embodiment comprises cyclosporin A, Alfonic TDA-3®, and Rapeseed oil.

Another embodiment comprises cyclosporin A, Alfonic TDA-4®, and Rapeseed oil.

Another embodiment comprises cyclosporin A, Alfonic TDA-6®, and Rapeseed oil.

Another embodiment comprises cyclosporin A, Alfonic TDA-7®, and Rapeseed oil.

Another embodiment comprises cyclosporin A, Alfonic TDA-8®, and Rapeseed oil.

Another embodiment comprises cyclosporin A, Alfonic TDA-9®, and Rapeseed oil.

Another embodiment comprises cyclosporin A, Alkamuls 400-DO®, and Rapeseed oil.

Another embodiment comprises cyclosporin A, Alkamuls 400-MO/E®, and Rapeseed oil.

Another embodiment comprises cyclosporin A, Ameroxol OE-10®, and Rapeseed oil.

Another embodiment comprises cyclosporin A, Ameroxol OE-2®, and Rapeseed oil.

Another embodiment comprises cyclosporin A, Ameroxol OE-20®, and Rapeseed oil.

Another embodiment comprises cyclosporin A, Armix 176®, and Rapeseed oil.

Another embodiment comprises cyclosporin A, Armix 180-C®, and Rapeseed oil.

Another embodiment comprises cyclosporin A, Armix 183®, and Rapeseed oil.

Another embodiment comprises cyclosporin A, Brij 35®, and Rapeseed oil:

Another embodiment comprises cyclosporin A, Brij 35 Liquids®, and Rapeseed oil.

Another embodiment comprises cyclosporin A, Brij 35 SP®, and Rapeseed oil.

Another embodiment comprises cyclosporin A, Canasol MJ 2109®, and Rapeseed oil.

Another embodiment comprises cyclosporin A, Canasol MJ 52®, and Rapeseed oil.

Another embodiment comprises cyclosporin A, Canasol R 3600®, and Rapeseed oil.

Another embodiment comprises cyclosporin A, Canasol R 3603®, and Rapeseed oil.

Another embodiment comprises cyclosporin A, Canasol R 4000 H®, and Rapeseed oil.

Another embodiment comprises cyclosporin A, Cedepal CA-210®, and Rapeseed oil.

Another embodiment comprises cyclosporin A, Cedepal CA-520®, and Rapeseed oil.

Another embodiment comprises cyclosporin A, Cedepal CA-630®, and Rapeseed oil.

Another embodiment comprises cyclosporin A, Cedepal CA-720®, and Rapeseed oil.

Another embodiment comprises cyclosporin A, Cedepal CA-890®, and Rapeseed oil.

Another embodiment comprises cyclosporin A, Cedepal CA-897®, and Rapeseed oil.

Another embodiment comprises cyclosporin A, Cedepal CO-210®, and Rapeseed oil.

Another embodiment comprises cyclosporin A, Cedepal CO-430®, and Rapeseed oil.

Another embodiment comprises cyclosporin A, Cedepal CO-530®, and Rapeseed oil.

Another embodiment comprises cyclosporin A, Cedepal CO-630®, and Rapeseed oil.

Another embodiment comprises cyclosporin A, Cedepal CO-710®, and Rapeseed oil.

Another embodiment comprises cyclosporin A, Cedepal CO-730®, and Rapeseed oil.

Another embodiment comprises cyclosporin A, Cedepal CO-880®, and Rapeseed oil.

Another embodiment comprises cyclosporin A, Cedepal CO-887®, and Rapeseed oil.

Another embodiment comprises cyclosporin A, Cedepal CO-890®, and Rapeseed oil.

Another embodiment comprises cyclosporin A, Cedepal CO-897®, and Rapeseed oil.

Another embodiment comprises cyclosporin A, Cedepal CO-970®, and Rapeseed oil.

Another embodiment comprises cyclosporin A, Cedepal CO-977®, and Rapeseed oil.

Another embodiment comprises cyclosporin A, Cedepal CO-990®, and Rapeseed oil.

Another embodiment comprises cyclosporin A, Cedepal CO-997®, and Rapeseed oil.

Another embodiment comprises cyclosporin A, Chemal BP 261®, and Rapeseed oil.

Another embodiment comprises cyclosporin A, Chemal BP 262®, and Rapeseed oil.

Another embodiment comprises cyclosporin A, Chemal BP 3172®, and Rapeseed oil.

Another embodiment comprises cyclosporin A, Chemal BP 3174®, and Rapeseed oil.

Another embodiment comprises cyclosporin A, Chemal DA-4®, and Rapeseed oil.

Another embodiment comprises cyclosporin A, Chemal DA-6®, and Rapeseed oil.

Another embodiment comprises cyclosporin A, Chemal DA-9®, and Rapeseed oil.

Another embodiment comprises cyclosporin A, Chemal LA-23®, and Rapeseed oil.

Another embodiment comprises cyclosporin A, Chemal LA-4®, and Rapeseed oil.

Another embodiment comprises cyclosporin A, Chemal LA-9®, and Rapeseed oil.

Another embodiment comprises cyclosporin A, Chemal OA-20G®, and Rapeseed oil.

Another embodiment comprises cyclosporin A, Chemal OA-5®, and Rapeseed oil.

Another embodiment comprises cyclosporin A, Chemal OA-9®, and Rapeseed oil.

Another embodiment comprises cyclosporin A, Chemal TDA-12®, and Rapeseed oil.

Another embodiment comprises cyclosporin A, Chemal TDA-15®, and Rapeseed oil.

Another embodiment comprises cyclosporin A, Chemal TDA-3®, and Rapeseed oil.

Another embodiment comprises cyclosporin A, Chemal TDA-6®, and Rapeseed oil.

Another embodiment comprises cyclosporin A, Chemal TDA-9®, and Rapeseed oil.

Another embodiment comprises cyclosporin A, Chemax CO-16®, and Rapeseed oil.

Another embodiment comprises cyclosporin A, Chemax CO-200/50®, and Rapeseed oil.

Another embodiment comprises cyclosporin A, Chemax CO-25®, and Rapeseed oil.

Another embodiment comprises cyclosporin A, Chemax CO-30®, and Rapeseed oil.

Another embodiment comprises cyclosporin A, Chemax CO-36®, and Rapeseed oil.

Another embodiment comprises cyclosporin A, Chemax CO-40®, and Rapeseed oil.

Another embodiment comprises cyclosporin A, Chemax CO-5®, and Rapeseed oil.

Another embodiment comprises cyclosporin A, Chemax CO-80®, and Rapeseed oil.

Another embodiment comprises cyclosporin A, Chemax DNP-150/50®, and Rapeseed oil.

Another embodiment comprises cyclosporin A, Chemax DNP-18®, and Rapeseed oil.

Another embodiment comprises cyclosporin A, Chemax DNP-8®, and Rapeseed oil.

Another embodiment comprises cyclosporin A, Chemax E 200 ML®, and Rapeseed oil.

Another embodiment comprises cyclosporin A, Chemax E 200 MO®, and Rapeseed oil.

Another embodiment comprises cyclosporin A, Chemax E 400 ML®, and Rapeseed oil.

Another embodiment comprises cyclosporin A, Chemax E 400 MO®, and Rapeseed oil.

Another embodiment comprises cyclosporin A, Chemax E 600 ML®, and Rapeseed oil.

Another embodiment comprises cyclosporin A, Chemax E 600 MO®, and Rapeseed oil.

Another embodiment comprises cyclosporin A, Chemax E 1000 MO®, and Rapeseed oil.

Another embodiment comprises cyclosporin A, Chemax E 1000 MS®, and Rapeseed oil.

Another embodiment comprises cyclosporin A, Chemax E200 MS®, and Rapeseed oil.

Another embodiment comprises cyclosporin A, Chemax E400 MS®, and Rapeseed oil.

Another embodiment comprises cyclosporin A, Chemax E600 MS®, and Rapeseed oil.

Another embodiment comprises cyclosporin A, Chemax HCO-16®, and Rapeseed oil.

Another embodiment comprises cyclosporin A, Chemax HCO-200/50®, and Rapeseed oil.

Another embodiment comprises cyclosporin A, Chemax HCO-25®, and Rapeseed oil.

Another embodiment comprises cyclosporin A, Chemax HCO-5®, and Rapeseed oil.

Another embodiment comprises cyclosporin A, Chemax TO-16®, and Rapeseed oil.

Another embodiment comprises cyclosporin A, Chemax TO-8®, and Rapeseed oil.

Another embodiment comprises cyclosporin A, Colalube 3409®, and Rapeseed oil.

Another embodiment comprises cyclosporin A, Colalube 3411®, and Rapeseed oil.

Another embodiment comprises cyclosporin A, Colalube 3413®, and Rapeseed oil.

Another embodiment comprises cyclosporin A, Colalube 3414®, and Rapeseed oil.

Another embodiment comprises cyclosporin A, Colalube 3415®, and Rapeseed oil.

Another embodiment comprises cyclosporin A, Colalube 3416®, and Rapeseed oil.

Another embodiment comprises cyclosporin A, Colalube 3417®, and Rapeseed oil.

Another embodiment comprises cyclosporin A, Delonic NPE-100®, and Rapeseed oil.

Another embodiment comprises cyclosporin A, Delonic NPE-30®, and Rapeseed oil.

Another embodiment comprises cyclosporin A, Delonic NPE-40®, and Rapeseed oil.

Another embodiment comprises cyclosporin A, Delonic OPE-10®, and Rapeseed oil.

Another embodiment comprises cyclosporin A, Delonic OPE-12®, and Rapeseed oil.

Another embodiment comprises cyclosporin A, Delonic OPE-30®, and Rapeseed oil.

Another embodiment comprises cyclosporin A, Delonic OPE-40®, and Rapeseed oil.

Another embodiment comprises cyclosporin A, Delonic OPE-5®, and Rapeseed oil.

Another embodiment comprises cyclosporin A, Delonic OPE-7.5®, and Rapeseed oil.

Another embodiment comprises cyclosporin A, DePeg 16-CO®, and Rapeseed oil.

Another embodiment comprises cyclosporin A, DePeg 25-CO®, and Rapeseed oil.

Another embodiment comprises cyclosporin A, DePeg 30-CO®, and Rapeseed oil.

Another embodiment comprises cyclosporin A, DePeg 40-CO®, and Rapeseed oil.

Another embodiment comprises cyclosporin A, DePeg 5-CO®, and Rapeseed oil.

Another embodiment comprises cyclosporin A, DePeg 80-CO®, and Rapeseed oil.

Another embodiment comprises cyclosporin A, DeSonic S-100®, and Rapeseed oil.

Another embodiment comprises cyclosporin A, DeSonic S-114®, and Rapeseed oil.

Another embodiment comprises cyclosporin A, DeSonic S-405®, and Rapeseed oil.

Another embodiment comprises cyclosporin A, DeSonic S-45®, and Rapeseed oil.

Another embodiment comprises cyclosporin A, DeSonic TDA-9®, and Rapeseed oil.

Another embodiment comprises cyclosporin A, DeThox Acid O-14®, and Rapeseed oil.

Another embodiment comprises cyclosporin A, DeThox Acid O-16®, and Rapeseed oil.

Another embodiment comprises cyclosporin A, DeThox Acid O-9®, and Rapeseed oil.

Another embodiment comprises cyclosporin A, DeThox Acid TO-14®, and Rapeseed oil.

Another embodiment comprises cyclosporin A, DeThox Acid TO-16.5®, and Rapeseed oil.

Another embodiment comprises cyclosporin A, DeThox Acid TO-8.5®, and Rapeseed oil.

Another embodiment comprises cyclosporin A, Dur-Em 114®, and Rapeseed oil.

Another embodiment comprises cyclosporin A, Dur-Em 117®, and Rapeseed oil.

Another embodiment comprises cyclosporin A, Dur-Em 204®, and Rapeseed oil.

Another embodiment comprises cyclosporin A, Dur-Em 207®, and Rapeseed oil.

Another embodiment comprises cyclosporin A, Dur-Em 207E®, and Rapeseed oil.

Another embodiment comprises cyclosporin A, Emerest 2704®, and Rapeseed oil.

Another embodiment comprises cyclosporin A, Emerest 2712®, and Rapeseed oil.

Another embodiment comprises cyclosporin A, Emerest 2715®, and Rapeseed oil.

Another embodiment comprises cyclosporin A, Ethal EH-2®, and Rapeseed oil.

Another embodiment comprises cyclosporin A, Ethal EH-5®, and Rapeseed oil.

Another embodiment comprises cyclosporin A, Ethal OA-23®, and Rapeseed oil.

Another embodiment comprises cyclosporin A, Ethosperse CA-20®, and Rapeseed oil.

Another embodiment comprises cyclosporin A, Ethosperse LA-12®, and Rapeseed oil.

Another embodiment comprises cyclosporin A, Ethosperse LA-23®, and Rapeseed oil.

Another embodiment comprises cyclosporin A, Ethosperse LA-4®, and Rapeseed oil.

Another embodiment comprises cyclosporin A, Ethox 3095®, and Rapeseed oil.

Another embodiment comprises cyclosporin A, Ethox CO-16®, and Rapeseed oil.

Another embodiment comprises cyclosporin A, Ethox CO-200®, and Rapeseed oil.

Another embodiment comprises cyclosporin A, Ethox CO-25®, and Rapeseed oil.

Another embodiment comprises cyclosporin A, Ethox CO-30®, and Rapeseed oil.

Another embodiment comprises cyclosporin A, Ethox CO-36®, and Rapeseed oil.

Another embodiment comprises cyclosporin A, Ethox CO-40®, and Rapeseed oil.

Another embodiment comprises cyclosporin A, Ethox CO-5®, and Rapeseed oil.

Another embodiment comprises cyclosporin A, Ethox DO-14®, and Rapeseed oil.

Another embodiment comprises cyclosporin A, Ethox DO-9®, and Rapeseed oil.

Another embodiment comprises cyclosporin A, Ethox MA-8®, and Rapeseed oil.

Another embodiment comprises cyclosporin A, Ethox MI-14®, and Rapeseed oil.

Another embodiment comprises cyclosporin A, Ethox ML-14®, and Rapeseed oil.

Another embodiment comprises cyclosporin A, Ethox ML-5®, and Rapeseed oil.

Another embodiment comprises cyclosporin A, Ethox MO-14®, and Rapeseed oil.

Another embodiment comprises cyclosporin A, Ethox MO-9®, and Rapeseed oil.

Another embodiment comprises cyclosporin A, Ethox MS-14®, and Rapeseed oil.

Another embodiment comprises cyclosporin A, Ethox MS-23®, and Rapeseed oil.

Another embodiment comprises cyclosporin A, Ethox MS-40®, and Rapeseed oil.

Another embodiment comprises cyclosporin A, Ethox MS-8®, and Rapeseed oil.

Another embodiment comprises cyclosporin A, Ethox TO-16®, and Rapeseed oil.

Another embodiment comprises cyclosporin A, Ethox TO-8®, and Rapeseed oil.

Another embodiment comprises cyclosporin A, Ethylan SN-120®, and Rapeseed oil.

Another embodiment comprises cyclosporin A, Ethylan SN-70®, and Rapeseed oil.

Another embodiment comprises cyclosporin A, Ethylan SN-90®, and Rapeseed oil.

Another embodiment comprises cyclosporin A, Flexricin 13®, and Rapeseed oil.

Another embodiment comprises cyclosporin A, Flexricin 15®, and Rapeseed oil.

Another embodiment comprises cyclosporin A, Genapol 1261®, and Rapeseed oil.

Another embodiment comprises cyclosporin A, Generol 122 E25®, and Rapeseed oil.

Another embodiment comprises cyclosporin A, Glicepol 160®, and Rapeseed oil.

Another embodiment comprises cyclosporin A, Glicepol 560®, and Rapeseed oil.

Another embodiment comprises cyclosporin A, Glicepol GMS 20®, and Rapeseed oil.

Another embodiment comprises cyclosporin A, Hetoxide BN-13®, and Rapeseed oil.

Another embodiment comprises cyclosporin A, Hetoxide DNP-4®, and Rapeseed oil.

Another embodiment comprises cyclosporin A, Hetoxide DNP-9.6®, and Rapeseed oil.

Another embodiment comprises cyclosporin A, Hetoxide G-26®, and Rapeseed oil.

Another embodiment comprises cyclosporin A, Hetoxide G-7®, and Rapeseed oil.

Another embodiment comprises cyclosporin A, Hetoxide GT-20®, and Rapeseed oil.

Another embodiment comprises cyclosporin A, Hetoxide HC-40®, and Rapeseed oil.

Another embodiment comprises cyclosporin A, Hetoxol C-24®, and Rapeseed oil.

Another embodiment comprises cyclosporin A, Hetoxol CA-10®, and Rapeseed oil.

Another embodiment comprises cyclosporin A, Hetoxol CA-2®, and Rapeseed oil.

Another embodiment comprises cyclosporin A, Hetoxol CA-20®, and Rapeseed oil.

Another embodiment comprises cyclosporin A, Hetoxol CAWS®, and Rapeseed oil.

Another embodiment comprises cyclosporin A, Hetoxol CD-3®, and Rapeseed oil.

Another embodiment comprises cyclosporin A, Hetoxol CD-4®, and Rapeseed oil.

Another embodiment comprises cyclosporin A, Hetoxol CS-15®, and Rapeseed oil.

Another embodiment comprises cyclosporin A, Hetoxol CS-20®, and Rapeseed oil.

Another embodiment comprises cyclosporin A, Hetoxol CS-20D®, and Rapeseed oil.

Another embodiment comprises cyclosporin A, Hetoxol CS-25®, and Rapeseed oil.

Another embodiment comprises cyclosporin A, Hetoxol CS-50®, and Rapeseed oil.

Another embodiment comprises cyclosporin A, Hetoxol CS-9®, and Rapeseed oil.

Another embodiment comprises cyclosporin A, Hetoxol CSA-15®, and Rapeseed oil.

Another embodiment comprises cyclosporin A, Hetoxol D®, and Rapeseed oil.

Another embodiment comprises cyclosporin A, Hetoxol G®, and Rapeseed oil.

Another embodiment comprises cyclosporin A, Hetoxol J®, and Rapeseed oil.

Another embodiment comprises cyclosporin A, Hetoxol L®, and Rapeseed oil.

Another embodiment comprises cyclosporin A, Hetoxol L-23®, and Rapeseed oil.

Another embodiment comprises cyclosporin A, Hetoxol L-4®, and Rapeseed oil.

Another embodiment comprises cyclosporin A, Hetoxol L-9®, and Rapeseed oil.

Another embodiment comprises cyclosporin A, Hetoxol LS-9®, and Rapeseed oil.

Another embodiment comprises cyclosporin A, Hetoxol M-3®, and Rapeseed oil.

Another embodiment comprises cyclosporin A, Hetoxol OA-10 Special®, and Rapeseed oil.

Another embodiment comprises cyclosporin A, Hetoxol OA-20 Special®, and Rapeseed oil.

Another embodiment comprises cyclosporin A, Hetoxol OA-3 Special®, and Rapeseed oil.

Another embodiment comprises cyclosporin A, Hetoxol OA-5 Special®, and Rapeseed oil.

Another embodiment comprises cyclosporin A, Hetoxol OL-10®, and Rapeseed oil.

Another embodiment comprises cyclosporin A, Hetoxol OL-2®, and Rapeseed oil.

Another embodiment comprises cyclosporin A, Hetoxol OL-23®, and Rapeseed oil.

Another embodiment comprises cyclosporin A, Hetoxol OL-4®, and Rapeseed oil.

Another embodiment comprises cyclosporin A, Hetoxol OL-40®, and Rapeseed oil.

Another embodiment comprises cyclosporin A, Hetoxol OL-5®, and Rapeseed oil.

Another embodiment comprises cyclosporin A, Hetoxol PLAP®, and Rapeseed oil.

Another embodiment comprises cyclosporin A, Hetoxol SP-15®, and Rapeseed oil.

Another embodiment comprises cyclosporin A, Hetoxol STA-10®, and Rapeseed oil.

Another embodiment comprises cyclosporin A, Hetoxol STA-2®, and Rapeseed oil.

Another embodiment comprises cyclosporin A, Hetoxol STA-30®, and Rapeseed oil.

Another embodiment comprises cyclosporin A, Hetoxol TD-12®, and Rapeseed oil.

Another embodiment comprises cyclosporin A, Hetoxol TD-18®, and Rapeseed oil.

Another embodiment comprises cyclosporin A, Hetoxol TD-3®, and Rapeseed oil.

Another embodiment comprises cyclosporin A, Hetoxol TD-6®, and Rapeseed oil.

Another embodiment comprises cyclosporin A, Hetoxol TD-9®, and Rapeseed oil.

Another embodiment comprises cyclosporin A, Hetoxol TDEP-15®, and Rapeseed oil.

Another embodiment comprises cyclosporin A, Hetoxol TDEP-63®, and Rapeseed oil.

Another embodiment comprises cyclosporin A, Iconol 24-12®, and Rapeseed oil.

Another embodiment comprises cyclosporin A, Iconol 24-23®, and Rapeseed oil.

Another embodiment comprises cyclosporin A, Iconol 24-4®, and Rapeseed oil.

Another embodiment comprises cyclosporin A, Iconol DA-4®, and Rapeseed oil.

Another embodiment comprises cyclosporin A, Iconol DA-6®, and Rapeseed oil.

Another embodiment comprises cyclosporin A, Iconol TDA-10®, and Rapeseed oil.

Another embodiment comprises cyclosporin A, Iconol TDA-3®, and Rapeseed oil.

Another embodiment comprises cyclosporin A, Iconol TDA-6®, and Rapeseed oil.

Another embodiment comprises cyclosporin A, Iconol TDA-8-90%®, and Rapeseed oil.

Another embodiment comprises cyclosporin A, Iconol TDA-9®, and Rapeseed oil.

Another embodiment comprises cyclosporin A, Igepal CA-210®, and Rapeseed oil.

Another embodiment comprises cyclosporin A, Igepal CA-407®, and Rapeseed oil.

Another embodiment comprises cyclosporin A, Igepal CA-420®, and Rapeseed oil.

Another embodiment comprises cyclosporin A, Igepal CA-520®, and Rapeseed oil.

Another embodiment comprises cyclosporin A, Igepal CA-620®, and Rapeseed oil.

Another embodiment comprises cyclosporin A, Igepal CA-630®, and Rapeseed oil.

Another embodiment comprises cyclosporin A, Igepal CA-720®, and Rapeseed oil.

Another embodiment comprises cyclosporin A, Igepal CA-877®, and Rapeseed oil.

Another embodiment comprises cyclosporin A, Igepal CA-887®, and Rapeseed oil.

Another embodiment comprises cyclosporin A, Igepal CA-897®, and Rapeseed oil.

Another embodiment comprises cyclosporin A, Igepal CO-210®, and Rapeseed oil.

Another embodiment comprises cyclosporin A, Igepal CO-430®, and Rapeseed oil.

Another embodiment comprises cyclosporin A, Igepal CO-520®, and Rapeseed oil.

Another embodiment comprises cyclosporin A, Igepal CO-530®, and Rapeseed oil.

Another embodiment comprises cyclosporin A, Igepal CO-610®, and Rapeseed oil.

Another embodiment comprises cyclosporin A, Igepal CO-630®, and Rapeseed oil.

Another embodiment comprises cyclosporin A, Igepal CO-630 Special®, and Rapeseed oil.

Another embodiment comprises cyclosporin A, Igepal CO-660®, and Rapeseed oil.

Another embodiment comprises cyclosporin A, Igepal CO-710®, and Rapeseed oil.

Another embodiment comprises cyclosporin A, Igepal CO-720®, and Rapeseed oil.

Another embodiment comprises cyclosporin A, Igepal CO-730®, and Rapeseed oil.

Another embodiment comprises cyclosporin A, Igepal CO-738®, and Rapeseed oil.

Another embodiment comprises cyclosporin A, Igepal CO-850®, and Rapeseed oil.

Another embodiment comprises cyclosporin A, Igepal CO-880®, and Rapeseed oil.

Another embodiment comprises cyclosporin A, Igepal CO-887®, and Rapeseed oil.

Another embodiment comprises cyclosporin A, Igepal CO-890®, and Rapeseed oil.

Another embodiment comprises cyclosporin A, Igepal CO-897®, and Rapeseed oil.

Another embodiment comprises cyclosporin A, Igepal CO-970®, and Rapeseed oil.

Another embodiment comprises cyclosporin A, Igepal CO-977®, and Rapeseed oil.

Another embodiment comprises cyclosporin A, Igepal CO-987®, and Rapeseed oil.

Another embodiment comprises cyclosporin A, Igepal CO-990 FLK®, and Rapeseed oil.

Another embodiment comprises cyclosporin A, Igepal CO-997®, and Rapeseed oil.

Another embodiment comprises cyclosporin A, Igepal DM-430®, and Rapeseed oil.

Another embodiment comprises cyclosporin A, Igepal DM-530®, and Rapeseed oil.

Another embodiment comprises cyclosporin A, Igepal DM-710®, and Rapeseed oil.

Another embodiment comprises cyclosporin A, Igepal DM-730®, and Rapeseed oil.

Another embodiment comprises cyclosporin A, Igepal DM-880®, and Rapeseed oil.

Another embodiment comprises cyclosporin A, Igepal DM-970 FLK®, and Rapeseed oil.

Another embodiment comprises cyclosporin A, Igepal OD-410®, and Rapeseed oil.

Another embodiment comprises cyclosporin A, Igepal RC-520®, and Rapeseed oil.

Another embodiment comprises cyclosporin A, Igepal RC-620®, and Rapeseed oil.

Another embodiment comprises cyclosporin A, Igepal RC-630®, and Rapeseed oil.

Another embodiment comprises cyclosporin A, Lexemul CS-20®, and Rapeseed oil.

Another embodiment comprises cyclosporin A, Lipo GMS 450®, and Rapeseed oil.

Another embodiment comprises cyclosporin A, Lipo Polyglycol 1000®, and Rapeseed oil.

Another embodiment comprises cyclosporin A, Lipo Polyglycol 200®, and Rapeseed oil.

Another embodiment comprises cyclosporin A, Lipo Polyglycol 300®, and Rapeseed oil.

Another embodiment comprises cyclosporin A, Lipo Polyglycol 3350®, and Rapeseed oil.

Another embodiment comprises cyclosporin A, Lipo Polyglycol 400®, and Rapeseed oil.

Another embodiment comprises cyclosporin A, Lipo Polyglycol 600®, and Rapeseed oil.

Another embodiment comprises cyclosporin A, Lipocol C-10®, and Rapeseed oil.

Another embodiment comprises cyclosporin A, Lipocol C-2®, and Rapeseed oil.

Another embodiment comprises cyclosporin A, Lipocol C-20®, and Rapeseed oil.

Another embodiment comprises cyclosporin A, Lipocol L-12®, and Rapeseed oil.

Another embodiment comprises cyclosporin A, Lipocol L-23®, and Rapeseed oil.

Another embodiment comprises cyclosporin A, Lipocol L-4®, and Rapeseed oil.

Another embodiment comprises cyclosporin A, Lipocol O-10®, and Rapeseed oil.

Another embodiment comprises cyclosporin A, Lipocol O-2®, and Rapeseed oil.

Another embodiment comprises cyclosporin A, Lipocol O-20®, and Rapeseed oil.

Another embodiment comprises cyclosporin A, Lipocol O-5®, and Rapeseed oil.

Another embodiment comprises cyclosporin A, Lipocol S-10®, and Rapeseed oil.

Another embodiment comprises cyclosporin A, Lipocol S-2®, and Rapeseed oil.

Another embodiment comprises cyclosporin A, Lipocol S-20®, and Rapeseed oil.

Another embodiment comprises cyclosporin A, Lipocol SC-15®, and Rapeseed oil.

Another embodiment comprises cyclosporin A, Lipocol SC-20®, and Rapeseed oil.

Another embodiment comprises cyclosporin A, Lipocol SC-4®, and Rapeseed oil.

Another embodiment comprises cyclosporin A, Lipocol TD-12®, and Rapeseed oil.

Another embodiment comprises cyclosporin A, Lumulse 40-O K®, and Rapeseed oil.

Another embodiment comprises cyclosporin A, Lumulse 42-O K®, and Rapeseed oil.

Another embodiment comprises cyclosporin A, Lumulse GMT-40®, and Rapeseed oil.

Another embodiment comprises cyclosporin A, Lumulse PEG 1450®, and Rapeseed oil.

Another embodiment comprises cyclosporin A, Lumulse PEG 1450 NF®, and Rapeseed oil.

Another embodiment comprises cyclosporin A, Lumulse PEG 8000®, and Rapeseed oil.

Another embodiment comprises cyclosporin A, Lumulse PGO®, and Rapeseed oil.

Another embodiment comprises cyclosporin A, Lumulse POE (2) Oleyl Amine®, and Rapeseed oil.

Another embodiment comprises cyclosporin A, Magrabar PGO-104®, and Rapeseed oil.

Another embodiment comprises cyclosporin A, Mapeg 200 ML®, and Rapeseed oil.

Another embodiment comprises cyclosporin A, Mapeg 400 MO®, and Rapeseed oil.

Another embodiment comprises cyclosporin A, Mapeg 400 MOT®, and Rapeseed oil.

Another embodiment comprises cyclosporin A, Mapeg 400DO®, and Rapeseed oil.

Another embodiment comprises cyclosporin A, Mapeg 400DOT®, and Rapeseed oil.

Another embodiment comprises cyclosporin A, Mapeg 600DOT®, and Rapeseed oil.

Another embodiment comprises cyclosporin A, Marlox B 24/80®, and Rapeseed oil.

Another embodiment comprises cyclosporin A, Marlox FK 86®, and Rapeseed oil.

Another embodiment comprises cyclosporin A, Marlox MO 154®, and Rapeseed oil.

Another embodiment comprises cyclosporin A, Mazox CDA CG®, and Rapeseed oil.

Another embodiment comprises cyclosporin A, Monoglycerides Glyceryl Monestearate®, and Rapeseed oil.

Another embodiment comprises cyclosporin A, Myrj 45®, and Rapeseed oil.

Another embodiment comprises cyclosporin A, Myrj 52®, and Rapeseed oil.

Another embodiment comprises cyclosporin A, Myrj 52S®, and Rapeseed oil.

Another embodiment comprises cyclosporin A, Myrj 53®, and Rapeseed oil.

Another embodiment comprises cyclosporin A, Myrj 59®, and Rapeseed oil.

Another embodiment comprises cyclosporin A, Norfox NP-12®, and Rapeseed oil.

Another embodiment comprises cyclosporin A, Norfox NP-4®, and Rapeseed oil.

Another embodiment comprises cyclosporin A, Norfox NP-6®, and Rapeseed oil.

Another embodiment comprises cyclosporin A, Norfox NP-887®, and Rapeseed oil.

Another embodiment comprises cyclosporin A, Norfox NP-9®, and Rapeseed oil.

Another embodiment comprises cyclosporin A, Norfox NP-977®, and Rapeseed oil.

Another embodiment comprises cyclosporin A, Norfox OP-100®, and Rapeseed oil.

Another embodiment comprises cyclosporin A, Norfox OP-102®, and Rapeseed oil.

Another embodiment comprises cyclosporin A, Norfox OP-114®, and Rapeseed oil.

Another embodiment comprises cyclosporin A, Norfox OP-45®, and Rapeseed oil.

Another embodiment comprises cyclosporin A, Pegosperse 100-L®, and Rapeseed oil.

Another embodiment comprises cyclosporin A, Pegosperse 100-O®, and Rapeseed oil.

Another embodiment comprises cyclosporin A, Pegosperse 100-S®, and Rapeseed oil.

Another embodiment comprises cyclosporin A, Pegosperse 1500-MS®, and Rapeseed oil.

Another embodiment comprises cyclosporin A, Pegosperse 1750-MS®, and Rapeseed oil.

Another embodiment comprises cyclosporin A, Pegosperse 200 DL®, and Rapeseed oil.

Another embodiment comprises cyclosporin A, Pegosperse 200-ML®, and Rapeseed oil.

Another embodiment comprises cyclosporin A, Pegosperse 400-DL®, and Rapeseed oil.

Another embodiment comprises cyclosporin A, Pegosperse 400-DO®, and Rapeseed oil.

Another embodiment comprises cyclosporin A, Pegosperse 400-DS®, and Rapeseed oil.

Another embodiment comprises cyclosporin A, Pegosperse 400-ML®, and Rapeseed oil.

Another embodiment comprises cyclosporin A, Pegosperse 400-MO®, and Rapeseed oil.

Another embodiment comprises cyclosporin A, Pegosperse 400-MOT®, and Rapeseed oil.

Another embodiment comprises cyclosporin A, Pegosperse 400-MS®, and Rapeseed oil.

Another embodiment comprises cyclosporin A, Pegosperse 50-DS®, and Rapeseed oil.

Another embodiment comprises cyclosporin A, Pegosperse 50-MS®, and Rapeseed oil.

Another embodiment comprises cyclosporin A, Pegosperse 600-ML®, and Rapeseed oil.

Another embodiment comprises cyclosporin A, Pegosperse 600-MS®, and Rapeseed oil.

Another embodiment comprises cyclosporin A, Plurafac A-38®, and Rapeseed oil.

Another embodiment comprises cyclosporin A, Plurafac B-25-5®, and Rapeseed oil.

Another embodiment comprises cyclosporin A, Plurafac B-26®, and Rapeseed oil.

Another embodiment comprises cyclosporin A, Plurafac D-25®, and Rapeseed oil.

Another embodiment comprises cyclosporin A, Plurafac LF 131®, and Rapeseed oil.

Another embodiment comprises cyclosporin A, Plurafac LF 4030®, and Rapeseed oil.

Another embodiment comprises cyclosporin A, Plurafac LF 7000®, and Rapeseed oil.

Another embodiment comprises cyclosporin A, Plurafac RA-20®, and Rapeseed oil.

Another embodiment comprises cyclosporin A, Plurafac RA-30®, and Rapeseed oil.

Another embodiment comprises cyclosporin A, Plurafac RA-40®, and Rapeseed oil.

Another embodiment comprises cyclosporin A, Pluronic 10R5®, and Rapeseed oil.

Another embodiment comprises cyclosporin A, Pluronic 17R2®, and Rapeseed oil.

Another embodiment comprises cyclosporin A, Pluronic 17R4®, and Rapeseed oil.

Another embodiment comprises cyclosporin A, Pluronic 25R2®, and Rapeseed oil.

Another embodiment comprises cyclosporin A, Pluronic 25R4®, and Rapeseed oil.

Another embodiment comprises cyclosporin A, Pluronic 31R1®, and Rapeseed oil.

Another embodiment comprises cyclosporin A, Pluronic F108®, and Rapeseed oil.

Another embodiment comprises cyclosporin A, Pluronic F127®, and Rapeseed oil.

Another embodiment comprises cyclosporin A, Pluronic F38®, and Rapeseed oil.

Another embodiment comprises cyclosporin A, Pluronic F68®, and Rapeseed oil.

Another embodiment comprises cyclosporin A, Pluronic F68LF®, and Rapeseed oil.

Another embodiment comprises cyclosporin A, Pluronic F77®, and Rapeseed oil.

Another embodiment comprises cyclosporin A, Pluronic F87®, and Rapeseed oil.

Another embodiment comprises cyclosporin A, Pluronic F88®, and Rapeseed oil.

Another embodiment comprises cyclosporin A, Pluronic F98®, and Rapeseed oil.

Another embodiment comprises cyclosporin A, Pluronic L10®, and Rapeseed oil.

Another embodiment comprises cyclosporin A, Pluronic L101®, and Rapeseed oil.

Another embodiment comprises cyclosporin A, Pluronic L121®, and Rapeseed oil.

Another embodiment comprises cyclosporin A, Pluronic L31®, and Rapeseed oil.

Another embodiment comprises cyclosporin A, Pluronic L35®, and Rapeseed oil.

Another embodiment comprises cyclosporin A, Pluronic L43®, and Rapeseed oil.

Another embodiment comprises cyclosporin A, Pluronic L44®, and Rapeseed oil.

Another embodiment comprises cyclosporin A, Pluronic L61®, and Rapeseed oil.

Another embodiment comprises cyclosporin A, Pluronic L62®, and Rapeseed oil.

Another embodiment comprises cyclosporin A, Pluronic L62D®, and Rapeseed oil.

Another embodiment comprises cyclosporin A, Pluronic L62LF®, and Rapeseed oil.

Another embodiment comprises cyclosporin A, Pluronic L64®, and Rapeseed oil.

Another embodiment comprises cyclosporin A, Pluronic L81®, and Rapeseed oil.

Another embodiment comprises cyclosporin A, Pluronic L92®, and Rapeseed oil.

Another embodiment comprises cyclosporin A, Pluronic P103®, and Rapeseed oil.

Another embodiment comprises cyclosporin A, Pluronic P104®, and Rapeseed oil.

Another embodiment comprises cyclosporin A, Pluronic P105®, and Rapeseed oil.

Another embodiment comprises cyclosporin A, Pluronic P123®, and Rapeseed oil.

Another embodiment comprises cyclosporin A, Pluronic P65®, and Rapeseed oil.

Another embodiment comprises cyclosporin A, Pluronic P84®, and Rapeseed oil.

Another embodiment comprises cyclosporin A, Pluronic P85®, and Rapeseed oil.

Another embodiment comprises cyclosporin A, Polyaldo 10-2-P®, and Rapeseed oil.

Another embodiment comprises cyclosporin A, Polyaldo DGDO®, and Rapeseed oil.

Another embodiment comprises cyclosporin A, Polyaldo HGDS®, and Rapeseed oil.

Another embodiment comprises cyclosporin A, Polyaldo TGMS®, and Rapeseed oil.

Another embodiment comprises cyclosporin A, Protegin V®, and Rapeseed oil.

Another embodiment comprises cyclosporin A, Protegin W®, and Rapeseed oil.

Another embodiment comprises cyclosporin A, Protegin WX®, and Rapeseed oil.

Another embodiment comprises cyclosporin A, Protegin XV®, and Rapeseed oil.

Another embodiment comprises cyclosporin A, Rhodasun BC-420®, and Rapeseed oil.

Another embodiment comprises cyclosporin A, Rhodasur BC-610®, and Rapeseed oil.

Another embodiment comprises cyclosporin A, Rhodasurf BC-630®, and Rapeseed oil.

Another embodiment comprises cyclosporin A, Rhodasurf BC-720®, and Rapeseed oil.

Another embodiment comprises cyclosporin A, Rhodasurf BC-840®, and Rapeseed oil.

Another embodiment comprises cyclosporin A, Rhodasurf L 1®, and Rapeseed oil.

Another embodiment comprises cyclosporin A, Rhodasurf L 12®, and Rapeseed oil.

Another embodiment comprises cyclosporin A, Rhodasurf L 2®, and Rapeseed oil.

Another embodiment comprises cyclosporin A, Rhodasurf L 20®, and Rapeseed oil.

Another embodiment comprises cyclosporin A, Rhodasurf L 3®, and Rapeseed oil.

Another embodiment comprises cyclosporin A, Rhodasurf L 30®, and Rapeseed oil.

Another embodiment comprises cyclosporin A, Rhodasurf L 4®, and Rapeseed oil.

Another embodiment comprises cyclosporin A, Rhodasurf L 50®, and Rapeseed oil.

Another embodiment comprises cyclosporin A, Rhodasurf L 7/90®, and Rapeseed oil.

Another embodiment comprises cyclosporin A, Rhodasurf LA-3®, and Rapeseed oil.

Another embodiment comprises cyclosporin A, Rhodasurf LA-7®, and Rapeseed oil.

Another embodiment comprises cyclosporin A, Rhodasurf LAN-23®, and Rapeseed oil.

Another embodiment comprises cyclosporin A, Rhodasurf ON-870®, and Rapeseed oil.

Another embodiment comprises cyclosporin A, Rhodasurf ON-877®, and Rapeseed oil.

Another embodiment comprises cyclosporin A, Rhodasurf TDA-8/5®, and Rapeseed oil.

Another embodiment comprises cyclosporin A, Ritapro 100®, and Rapeseed oil.

Another embodiment comprises cyclosporin A, Ritapro 165®, and Rapeseed oil.

Another embodiment comprises cyclosporin A, Ritapro 200®, and Rapeseed oil.

Another embodiment comprises cyclosporin A, Ritapro 300®, and Rapeseed oil.

Another embodiment comprises cyclosporin A, Ritoleth 10®, and Rapeseed oil.

Another embodiment comprises cyclosporin A, Ritoleth 2®, and Rapeseed oil.

Another embodiment comprises cyclosporin A, Ritoleth 20®, and Rapeseed oil.

Another embodiment comprises cyclosporin A, Ritoleth 5®, and Rapeseed oil.

Another embodiment comprises cyclosporin A, Santone 10-10-0®, and Rapeseed oil.

Another embodiment comprises cyclosporin A, Santone 3-1-S XTR®, and Rapeseed oil.

Another embodiment comprises cyclosporin A, Santone 3-1-SH®, and Rapeseed oil.

Another embodiment comprises cyclosporin A, Santone 8-1-O®, and Rapeseed oil.

Another embodiment comprises cyclosporin A, Solulan 16®, and Rapeseed oil.

Another embodiment comprises cyclosporin A, Solulan 75®, and Rapeseed oil.

Another embodiment comprises cyclosporin A, Solulan C-24®, and Rapeseed oil.

Another embodiment comprises cyclosporin A, Solulan L-575®, and Rapeseed oil.

Another embodiment comprises cyclosporin A, Standamox CAW®, and Rapeseed oil.

Another embodiment comprises cyclosporin A, Surfine AZI-A®, and Rapeseed oil.

Another embodiment comprises cyclosporin A, Surfonic CO-15®, and Rapeseed oil.

Another embodiment comprises cyclosporin A, Surfonic CO-25®, and Rapeseed oil.

Another embodiment comprises cyclosporin A, Surfonic CO-30®, and Rapeseed oil.

Another embodiment comprises cyclosporin A, Surfonic CO-36®, and Rapeseed oil.

Another embodiment comprises cyclosporin A, Surfonic CO-42®, and Rapeseed oil.

Another embodiment comprises cyclosporin A, Surfonic E400-MO®, and Rapeseed oil.

Another embodiment comprises cyclosporin A, Surfonic L12-3®, and Rapeseed oil.

Another embodiment comprises cyclosporin A, Surfonic L12-6®, and Rapeseed oil.

Another embodiment comprises cyclosporin A, Surfonic L12-8®, and Rapeseed oil.

Another embodiment comprises cyclosporin A, Surfonic L24-12®, and Rapeseed oil.

Another embodiment comprises cyclosporin A, Surfonic L24-2®, and Rapeseed oil.

Another embodiment comprises cyclosporin A, Surfonic L24-22®, and Rapeseed oil.

Another embodiment comprises cyclosporin A, Surfonic L24-3®, and Rapeseed oil.

Another embodiment comprises cyclosporin A, Surfonic L24-4®, and Rapeseed oil.

Another embodiment comprises cyclosporin A, Surfonic L24-5®, and Rapeseed oil.

Another embodiment comprises cyclosporin A, Surfonic L24-7®, and Rapeseed oil.

Another embodiment comprises cyclosporin A, Surfonic L24-7.1®, and Rapeseed oil.

Another embodiment comprises cyclosporin A, Surfonic L24-9®, and Rapeseed oil.

Another embodiment comprises cyclosporin A, Surfonic LF-17®, and Rapeseed oil.

Another embodiment comprises cyclosporin A, Surfonic LF-18®, and Rapeseed oil.

Another embodiment comprises cyclosporin A, Surfonic LF-37®, and Rapeseed oil.

Another embodiment comprises cyclosporin A, Surfonic LF-41®, and Rapeseed oil.

Another embodiment comprises cyclosporin A, Surfonic LF-42®, and Rapeseed oil.

Another embodiment comprises cyclosporin A, Surfonic LF-47®, and Rapeseed oil.

Another embodiment comprises cyclosporin A, Surfonic LF-68®, and Rapeseed oil.

Another embodiment comprises cyclosporin A, Surfonic N-10®, and Rapeseed oil.

Another embodiment comprises cyclosporin A, Surfonic N-100®, and Rapeseed oil.

Another embodiment comprises cyclosporin A, Surfonic N-102®, and Rapeseed oil.

Another embodiment comprises cyclosporin A, Surfonic N-120®, and Rapeseed oil.

Another embodiment comprises cyclosporin A, Surfonic N-150®, and Rapeseed oil.

Another embodiment comprises cyclosporin A, Surfonic N-200®, and Rapeseed oil.

Another embodiment comprises cyclosporin A, Surfonic N-300®, and Rapeseed oil.

Another embodiment comprises cyclosporin A, Surfonic N-31.5®, and Rapeseed oil.

Another embodiment comprises cyclosporin A, Surfonic N-40®, and Rapeseed oil.

Another embodiment comprises cyclosporin A, Surfonic N-400®, and Rapeseed oil.

Another embodiment comprises cyclosporin A, Surfonic N-60®, and Rapeseed oil.

Another embodiment comprises cyclosporin A, Surfonic N-85®, and Rapeseed oil.

Another embodiment comprises cyclosporin A, Surfonic N-95®, and Rapeseed oil.

Another embodiment comprises cyclosporin A, Surfonic NB-18®, and Rapeseed oil.

Another embodiment comprises cyclosporin A, Surfonic NB-23®, and Rapeseed oil.

Another embodiment comprises cyclosporin A, Surfonic NB-25®, and Rapeseed oil.

Another embodiment comprises cyclosporin A, Surfonic NB-307®, and Rapeseed oil.

Another embodiment comprises cyclosporin A, Surfonic NB-31®, and Rapeseed oil.

Another embodiment comprises cyclosporin A, Surfonic NB-41®, and Rapeseed oil.

Another embodiment comprises cyclosporin A, Surfonic NB-43®, and Rapeseed oil.

Another embodiment comprises cyclosporin A, Surfonic OP-100®, and Rapeseed oil.

Another embodiment comprises cyclosporin A, Surfonic OP-120®, and Rapeseed oil.

Another embodiment comprises cyclosporin A, Surfonic OP-15®, and Rapeseed oil.

Another embodiment comprises cyclosporin A, Surfonic OP-35®, and Rapeseed oil.

Another embodiment comprises cyclosporin A, Surfonic OP-70®, and Rapeseed oil.

Another embodiment comprises cyclosporin A, Surfonic P1®, and Rapeseed oil.

Another embodiment comprises cyclosporin A, Surfonic P3®, and Rapeseed oil.

Another embodiment comprises cyclosporin A, Surfonic P5®, and Rapeseed oil.

Another embodiment comprises cyclosporin A, Surfonic P6®, and Rapeseed oil.

Another embodiment comprises cyclosporin A, Surfonic POA-17R2®, and Rapeseed oil.

Another embodiment comprises cyclosporin A, Surfonic POA-25R2®, and Rapeseed oil.

Another embodiment comprises cyclosporin A, Surfonic TDA-11®, and Rapeseed oil.

Another embodiment comprises cyclosporin A, Surfonic TDA-3B®, and Rapeseed oil.

Another embodiment comprises cyclosporin A, Surfonic TDA-6®, and Rapeseed oil.

Another embodiment comprises cyclosporin A, Surfonic TDA-6/88®, and Rapeseed oil.

Another embodiment comprises cyclosporin A, Surfonic TDA-8®, and Rapeseed oil.

Another embodiment comprises cyclosporin A, Surfonic TDA-8/90®, and Rapeseed oil.

Another embodiment comprises cyclosporin A, Surfonic TDA-9®, and Rapeseed oil.

Another embodiment comprises cyclosporin A, Surfonic TX-CCN®, and Rapeseed oil.

Another embodiment comprises cyclosporin A, Tagat CH 40®, and Rapeseed oil.

Another embodiment comprises cyclosporin A, Tagat CH 60®, and Rapeseed oil.

Another embodiment comprises cyclosporin A, Tagat L 2®, and Rapeseed oil.

Another embodiment comprises cyclosporin A, Tagat S®, and Rapeseed oil.

Another embodiment comprises cyclosporin A, Tagat S 2®, and Rapeseed oil.

Another embodiment comprises cyclosporin A, Tally 100 Plus®, and Rapeseed oil.

Another embodiment comprises cyclosporin A, T-Det A106®, and Rapeseed oil.

Another embodiment comprises cyclosporin A, T-Det A109®, and Rapeseed oil.

Another embodiment comprises cyclosporin A, T-Det A136®, and Rapeseed oil.

Another embodiment comprises cyclosporin A, T-Det A139®, and Rapeseed oil.

Another embodiment comprises cyclosporin A, T-Det A2412®, and Rapeseed oil.

Another embodiment comprises cyclosporin A, T-Det A243®, and Rapeseed oil.

Another embodiment comprises cyclosporin A, T-Det A247®, and Rapeseed oil.

Another embodiment comprises cyclosporin A, T-Det A249®, and Rapeseed oil.

Another embodiment comprises cyclosporin A, T-Det A467®, and Rapeseed oil.

Another embodiment comprises cyclosporin A, T-Det COE®, and Rapeseed oil.

Another embodiment comprises cyclosporin A, T-Det DD-10®, and Rapeseed oil.

Another embodiment comprises cyclosporin A, T-Det DD-5®, and Rapeseed oil.

Another embodiment comprises cyclosporin A, T-Det DD-7®, and Rapeseed oil.

Another embodiment comprises cyclosporin A, T-Det EPO-104®, and Rapeseed oil.

Another embodiment comprises cyclosporin A, T-Det EPO-61®, and Rapeseed oil.

Another embodiment comprises cyclosporin A, T-Det EPO-62®, and Rapeseed oil.

Another embodiment comprises cyclosporin A, T-Det EPO-64®, and Rapeseed oil.

Another embodiment comprises cyclosporin A, T-Det N-1.5®, and Rapeseed oil.

Another embodiment comprises cyclosporin A, T-Det N-10.5®, and Rapeseed oil.

Another embodiment comprises cyclosporin A, T-Det N-100®, and Rapeseed oil.

Another embodiment comprises cyclosporin A, T-Det N-1007®, and Rapeseed oil.

Another embodiment comprises cyclosporin A, T-Det N-12®, and Rapeseed oil.

Another embodiment comprises cyclosporin A, T-Det N-14®, and Rapeseed oil.

Another embodiment comprises cyclosporin A, T-Det N-20®, and Rapeseed oil.

Another embodiment comprises cyclosporin A, T-Det N-30®, and Rapeseed oil.

Another embodiment comprises cyclosporin A, T-Det N-307®, and Rapeseed oil.

Another embodiment comprises cyclosporin A, T-Det N-4®, and Rapeseed oil.

Another embodiment comprises cyclosporin A, T-Det N-40®, and Rapeseed oil.

Another embodiment comprises cyclosporin A, T-Det N-407®, and Rapeseed oil.

Another embodiment comprises cyclosporin A, T-Det N-50®, and Rapeseed oil.

Another embodiment comprises cyclosporin A, T-Det N-507®, and Rapeseed oil.

Another embodiment comprises cyclosporin A, T-Det N-6®, and Rapeseed oil.

Another embodiment comprises cyclosporin A, T-Det N-70®, and Rapeseed oil.

Another embodiment comprises cyclosporin A, T-Det N-8®, and Rapeseed oil.

Another embodiment comprises cyclosporin A, T-Det N-9.5®, and Rapeseed oil.

Another embodiment comprises cyclosporin A, T-Det O-12®, and Rapeseed oil.

Another embodiment comprises cyclosporin A, T-Det O-165®, and Rapeseed oil.

Another embodiment comprises cyclosporin A, T-Det O-307®, and Rapeseed oil.

Another embodiment comprises cyclosporin A, T-Det O-40®, and Rapeseed oil.

Another embodiment comprises cyclosporin A, T-Det O-407®, and Rapeseed oil.

Another embodiment comprises cyclosporin A, T-Det O-6®, and Rapeseed oil.

Another embodiment comprises cyclosporin A, T-Det O-8®, and Rapeseed oil.

Another embodiment comprises cyclosporin A, T-Det O-9®, and Rapeseed oil.

Another embodiment comprises cyclosporin A, Tegin®, and Rapeseed oil.

Another embodiment comprises cyclosporin A, Tergitol 15-S-12®, and Rapeseed oil.

Another embodiment comprises cyclosporin A, Tergitol 15-S-15®, and Rapeseed oil.

Another embodiment comprises cyclosporin A, Tergitol 15-S-20®, and Rapeseed oil.

Another embodiment comprises cyclosporin A, Tergitol 15-S-20 (80AQ)®, and Rapeseed oil.

Another embodiment comprises cyclosporin A, Tergitol 15-S-3®, and Rapeseed oil.

Another embodiment comprises cyclosporin A, Tergitol 15-S-30®, and Rapeseed oil.

Another embodiment comprises cyclosporin A, Tergitol 15-S-40®, and Rapeseed oil.

Another embodiment comprises cyclosporin A, Tergitol 15-S-40 (70AQ)®, and Rapeseed oil.

Another embodiment comprises cyclosporin A, Tergitol 15-S-5®, and Rapeseed oil.

Another embodiment comprises cyclosporin A, Tergitol 15-S-7®, and Rapeseed oil.

Another embodiment comprises cyclosporin A, Tergitol 15-S-9®, and Rapeseed oil.

Another embodiment comprises cyclosporin A, Tergitol L-101®, and Rapeseed oil.

Another embodiment comprises cyclosporin A, Tergitol L-61®, and Rapeseed oil.

Another embodiment comprises cyclosporin A, Tergitol L-62®, and Rapeseed oil.

Another embodiment comprises cyclosporin A, Tergitol L-64®, and Rapeseed oil.

Another embodiment comprises cyclosporin A, Tergitol L-81®, and Rapeseed oil.

Another embodiment comprises cyclosporin A, Tergitol NP-10®, and Rapeseed oil.

Another embodiment comprises cyclosporin A, Tergitol NP-11®, and Rapeseed oil.

Another embodiment comprises cyclosporin A, Tergitol NP-12®, and Rapeseed oil.

Another embodiment comprises cyclosporin A, Tergitol NP-13®, and Rapeseed oil.

Another embodiment comprises cyclosporin A, Tergitol NP-15®, and Rapeseed oil.

Another embodiment comprises cyclosporin A, Tergitol NP-30®, and Rapeseed oil.

Another embodiment comprises cyclosporin A, Tergitol NP-30 (70 AQ)®, and Rapeseed oil.

Another embodiment comprises cyclosporin A, Tergitol NP-4®, and Rapeseed oil.

Another embodiment comprises cyclosporin A, Tergitol NP-40®, and Rapeseed oil.

Another embodiment comprises cyclosporin A, Tergitol NP-40 (70 AQ)®, and Rapeseed oil.

Another embodiment comprises cyclosporin A, Tergitol NP-50 (70 AQ)®, and Rapeseed oil.

Another embodiment comprises cyclosporin A, Tergitol NP-55 (70 AQ)®, and Rapeseed oil.

Another embodiment comprises cyclosporin A, Tergitol NP-6®, and Rapeseed oil.

Another embodiment comprises cyclosporin A, Tergitol NP-7®, and Rapeseed oil.

Another embodiment comprises cyclosporin A, Tergitol NP-70 (70 AQ)®, and Rapeseed oil.

Another embodiment comprises cyclosporin A, Tergitol NP-8®, and Rapeseed oil.

Another embodiment comprises cyclosporin A, Tergitol NP-9®, and Rapeseed oil.

Another embodiment comprises cyclosporin A, Tergitol NP-9.5®, and Rapeseed oil.

Another embodiment comprises cyclosporin A, Tergitol TMN-10®, and Rapeseed oil.

Another embodiment comprises cyclosporin A, Tergitol TMN-3®, and Rapeseed oil.

Another embodiment comprises cyclosporin A, Tergitol TMN-6®, and Rapeseed oil.

Another embodiment comprises cyclosporin A, Tergitol XD®, and Rapeseed oil.

Another embodiment comprises cyclosporin A, Tergitol XH®, and Rapeseed oil.

Another embodiment comprises cyclosporin A, Tergitol XJ®, and Rapeseed oil.

Another embodiment comprises cyclosporin A, Tetronic 1107®, and Rapeseed oil.

Another embodiment comprises cyclosporin A, Tetronic 1307®, and Rapeseed oil.

Another embodiment comprises cyclosporin A, Tetronic 150R1®, and Rapeseed oil.

Another embodiment comprises cyclosporin A, Tetronic 304®, and Rapeseed oil.

Another embodiment comprises cyclosporin A, Tetronic 701®, and Rapeseed oil.

Another embodiment comprises cyclosporin A, Tetronic 901®, and Rapeseed oil.

Another embodiment comprises cyclosporin A, Tetronic 904®, and Rapeseed oil.

Another embodiment comprises cyclosporin A, Tetronic 908®, and Rapeseed oil.

Another embodiment comprises cyclosporin A, Tetronic 90R4®, and Rapeseed oil.

Another embodiment comprises cyclosporin A, Tomadol 1-3®, and Rapeseed oil.

Another embodiment comprises cyclosporin A, Tomadol 1-5®, and Rapeseed oil.

Another embodiment comprises cyclosporin A, Tomadol 1-7®, and Rapeseed oil.

Another embodiment comprises cyclosporin A, Tomadol 23-1®, and Rapeseed oil.

Another embodiment comprises cyclosporin A, Tomadol 23-3®, and Rapeseed oil.

Another embodiment comprises cyclosporin A, Tomadol 23-5®, and Rapeseed oil.

Another embodiment comprises cyclosporin A, Tomadol 23-6.5®, and Rapeseed oil.

Another embodiment comprises cyclosporin A, Tomadol 25-12®, and Rapeseed oil.

Another embodiment comprises cyclosporin A, Tomadol 25-3®, and Rapeseed oil.

Another embodiment comprises cyclosporin A, Tomadol 25-7®, and Rapeseed oil.

Another embodiment comprises cyclosporin A, Tomadol 25-9®, and Rapeseed oil.

Another embodiment comprises cyclosporin A, Tomadol 45-13®, and Rapeseed oil.

Another embodiment comprises cyclosporin A, Tomadol 45-2.5®, and Rapeseed oil.

Another embodiment comprises cyclosporin A, Tomadol 45-7®, and Rapeseed oil.

Another embodiment comprises cyclosporin A, Tomadol 91-2.5®, and Rapeseed oil.

Another embodiment comprises cyclosporin A, Tomadol 91-6®, and Rapeseed oil.

Another embodiment comprises cyclosporin A, Tomadol 91-8®, and Rapeseed oil.

Another embodiment comprises cyclosporin A, Triton X-100®, and Rapeseed oil.

Another embodiment comprises cyclosporin A, Triton X-102®, and Rapeseed oil.

Another embodiment comprises cyclosporin A, Triton X-114®, and Rapeseed oil.

Another embodiment comprises cyclosporin A, Triton X-15®, and Rapeseed oil.

Another embodiment comprises cyclosporin A, Triton X-165 (70%)®, and Rapeseed oil.

Another embodiment comprises cyclosporin A, Triton X-207®, and Rapeseed oil.

Another embodiment comprises cyclosporin A, Triton X-305 (70%)®, and Rapeseed oil.

Another embodiment comprises cyclosporin A, Triton X-35®, and Rapeseed oil.

Another embodiment comprises cyclosporin A, Triton X-405 (70%)®, and Rapeseed oil.

Another embodiment comprises cyclosporin A, Triton X-45®, and Rapeseed oil.

Another embodiment comprises cyclosporin A, Triton X-705 (70%)®, and Rapeseed oil.

Another embodiment comprises cyclosporin A, Volpo S-10®, and Rapeseed oil.

Another embodiment comprises cyclosporin A, Volpo S-2®, and Rapeseed oil.

Another embodiment comprises cyclosporin A, Volpo S-20®, and Rapeseed oil.

Another embodiment comprises cyclosporin A, Volpo-10®, and Rapeseed oil.

Another embodiment comprises cyclosporin A, Volpo-20®, and Rapeseed oil.

Another embodiment comprises cyclosporin A, Volpo-3®, and Rapeseed oil.

Another embodiment comprises cyclosporin A, Volpo-5®, and Rapeseed oil.

Another embodiment comprises cyclosporin A, Abex 2515®, and Soybean oil.

Another embodiment comprises cyclosporin A, Abex 2525/40®, and Soybean oil.

Another embodiment comprises cyclosporin A, Abex 2535®, and Soybean oil.

Another embodiment comprises cyclosporin A, Abex 2545®, and Soybean oil.

Another embodiment comprises cyclosporin A, Acconon E®, and Soybean oil.

Another embodiment comprises cyclosporin A, Alfonic 1216CO-9®, and Soybean oil.

Another embodiment comprises cyclosporin A, Alfonic 1412-3®, and Soybean oil.

Another embodiment comprises cyclosporin A, Alfonic TDA-12®, and Soybean oil.

Another embodiment comprises cyclosporin A, Alfonic TDA-3®, and Soybean oil.

Another embodiment comprises cyclosporin A, Alfonic TDA-4®, and Soybean oil.

Another embodiment comprises cyclosporin A, Alfonic TDA-6®, and Soybean oil.

Another embodiment comprises cyclosporin A, Alfonic TDA-7®, and Soybean oil.

Another embodiment comprises cyclosporin A, Alfonic TDA-8®, and Soybean oil.

Another embodiment comprises cyclosporin A, Alfonic TDA-9®, and Soybean oil.

Another embodiment comprises cyclosporin A, Alkamuls 400-DO®, and Soybean oil.

Another embodiment comprises cyclosporin A, Alkamuls 400-MO/E®, and Soybean oil.

Another embodiment comprises cyclosporin A, Ameroxol OE-10®, and Soybean oil.

Another embodiment comprises cyclosporin A, Ameroxol OE-2®, and Soybean oil.

Another embodiment comprises cyclosporin A, Ameroxol OE-20®, and Soybean oil.

Another embodiment comprises cyclosporin A, Armix 176®, and Soybean oil.

Another embodiment comprises cyclosporin A, Armix 180-C®, and Soybean oil.

Another embodiment comprises cyclosporin A, Armix 183®, and Soybean oil.

Another embodiment comprises cyclosporin A, Brij 35®, and Soybean oil.

Another embodiment comprises cyclosporin A, Brij 35 Liquid®, and Soybean oil.

Another embodiment comprises cyclosporin A, Brij 35 SP®, and Soybean oil.

Another embodiment comprises cyclosporin A, Canasol MJ 2109®, and Soybean oil.

Another embodiment comprises cyclosporin A, Canasol MJ 52®, and Soybean oil.

Another embodiment comprises cyclosporin A, Canasol R 3600®, and Soybean oil.

Another embodiment comprises cyclosporin A, Canasol R 3603®, and Soybean oil.

Another embodiment comprises cyclosporin A, Canasol R 4000 H®, and Soybean oil.

Another embodiment comprises cyclosporin A, Cedepal CA-210®, and Soybean oil.

Another embodiment comprises cyclosporin A, Cedepal CA-520®, and Soybean oil.

Another embodiment comprises cyclosporin A, Cedepal CA-630®, and Soybean oil.

Another embodiment comprises cyclosporin A, Cedepal CA-720®, and Soybean oil.

Another embodiment comprises cyclosporin A, Cedepal CA-890®, and Soybean oil.

Another embodiment comprises cyclosporin A, Cedepal CA-897®, and Soybean oil.

Another embodiment comprises cyclosporin A, Cedepal CO-210®, and Soybean oil.

Another embodiment comprises cyclosporin A, Cedepal CO-430®, and Soybean oil.

Another embodiment comprises cyclosporin A, Cedepal CO-530®, and Soybean oil.

Another embodiment comprises cyclosporin A, Cedepal CO-630®, and Soybean oil.

Another embodiment comprises cyclosporin A, Cedepal CO-710®, and Soybean oil.

Another embodiment comprises cyclosporin A, Cedepal CO-730®, and Soybean oil.

Another embodiment comprises cyclosporin A, Cedepal CO-880®, and Soybean oil.

Another embodiment comprises cyclosporin A, Cedepal CO-887®, and Soybean oil.

Another embodiment comprises cyclosporin A, Cedepal CO-890®, and Soybean oil.

Another embodiment comprises cyclosporin A, Cedepal CO-897®, and Soybean oil.

Another embodiment comprises cyclosporin A, Cedepal CO-970®, and Soybean oil.

Another embodiment comprises cyclosporin A, Cedepal CO-977®, and Soybean oil.

Another embodiment comprises cyclosporin A, Cedepal CO-990®, and Soybean oil.

Another embodiment comprises cyclosporin A, Cedepal CO-997®, and Soybean oil.

Another embodiment comprises cyclosporin A, Chemal BP 261®, and Soybean oil.

Another embodiment comprises cyclosporin A, Chemal BP 262®, and Soybean oil.

Another embodiment comprises cyclosporin A, Chemal BP 3172®, and Soybean oil.

Another embodiment comprises cyclosporin A, Chemal BP 3174®, and Soybean oil.

Another embodiment comprises cyclosporin A, Chemal DA-4®, and Soybean oil.

Another embodiment comprises cyclosporin A, Chemal DA-6®, and Soybean oil.

Another embodiment comprises cyclosporin A, Chemal DA-9®, and Soybean oil.

Another embodiment comprises cyclosporin A, Chemal LA-23®, and Soybean oil.

Another embodiment comprises cyclosporin A, Chemal LA-4®, and Soybean oil.

Another embodiment comprises cyclosporin A, Chemal LA-9®, and Soybean oil.

Another embodiment comprises cyclosporin A, Chemal OA-20G®, and Soybean oil.

Another embodiment comprises cyclosporin A, Chemal OA-5®, and Soybean oil.

Another embodiment comprises cyclosporin A, Chemal OA-9®, and Soybean oil.

Another embodiment comprises cyclosporin A, Chemal TDA-12®, and Soybean oil.

Another embodiment comprises cyclosporin A, Chemal TDA-15®, and Soybean oil.

Another embodiment comprises cyclosporin A, Chemal TDA-3®, and Soybean oil.

Another embodiment comprises cyclosporin A, Chemal TDA-6®, and Soybean oil.

Another embodiment comprises cyclosporin A, Chemal TDA-9®, and Soybean oil.

Another embodiment comprises cyclosporin A, Chemax CO-16®, and Soybean oil.

Another embodiment comprises cyclosporin A, Chemax CO-200/50®, and Soybean oil.

Another embodiment comprises cyclosporin A, Chemax CO-25®, and Soybean oil.

Another embodiment comprises cyclosporin A, Chemax CO-30®, and Soybean oil.

Another embodiment comprises cyclosporin A, Chemax CO-36®, and Soybean oil.

Another embodiment comprises cyclosporin A, Chemax CO-40®, and Soybean oil.

Another embodiment comprises cyclosporin A, Chemax CO-5®, and Soybean oil.

Another embodiment comprises cyclosporin A, Chemax CO-80®, and Soybean oil.

Another embodiment comprises cyclosporin A, Chemax DNP-150/50®, and Soybean oil.

Another embodiment comprises cyclosporin A, Chemax DNP-18®, and Soybean oil.

Another embodiment comprises cyclosporin A, Chemax DNP-8®, and Soybean oil.

Another embodiment comprises cyclosporin A, Chemax E 200 ML®, and Soybean oil.

Another embodiment comprises cyclosporin A, Chemax E 200 MO®, and Soybean oil.

Another embodiment comprises cyclosporin A, Chemax E 400 ML®, and Soybean oil.

Another embodiment comprises cyclosporin A, Chemax E 400 MO®, and Soybean oil.

Another embodiment comprises cyclosporin A, Chemax E 600 ML®, and Soybean oil.

Another embodiment comprises cyclosporin A, Chemax E 600 MO®, and Soybean oil.

Another embodiment comprises cyclosporin A, Chemax E 1000 MO®, and Soybean oil.

Another embodiment comprises cyclosporin A, Chemax E 1000 MS®, and Soybean oil.

Another embodiment comprises cyclosporin A, Chemax E200 MS®, and Soybean oil.

Another embodiment comprises cyclosporin A, Chemax E400 MS®, and Soybean oil.

Another embodiment comprises cyclosporin A, Chemax E600 MS®, and Soybean oil.

Another embodiment comprises cyclosporin A, Chemax HCO-16®, and Soybean oil.

Another embodiment comprises cyclosporin A, Chemax HCO-200/50®, and Soybean oil.

Another embodiment comprises cyclosporin A, Chemax HCO-25®, and Soybean oil.

Another embodiment comprises cyclosporin A, Chemax HCO-5®, and Soybean oil.

Another embodiment comprises cyclosporin A, Chemax TO-16®, and Soybean oil.

Another embodiment comprises cyclosporin A, Chemax TO-8®, and Soybean oil.

Another embodiment comprises cyclosporin A, Colalube 3409®, and Soybean oil.

Another embodiment comprises cyclosporin A, Colalube 3411®, and Soybean oil.

Another embodiment comprises cyclosporin A, Colalube 3413®, and Soybean oil.

Another embodiment comprises cyclosporin A, Colalube 3414®, and Soybean oil.

Another embodiment comprises cyclosporin A, Colalube 3415®, and Soybean oil.

Another embodiment comprises cyclosporin A, Colalube 3416®, and Soybean oil.

Another embodiment comprises cyclosporin A, Colalube 3417®, and Soybean oil.

Another embodiment comprises cyclosporin A, Delonic NPE-100®, and Soybean oil.

Another embodiment comprises cyclosporin A, Delonic NPE-30®, and Soybean oil.

Another embodiment comprises cyclosporin A, Delonic NPE-40®, and Soybean oil.

Another embodiment comprises cyclosporin A, Delonic OPE-10®, and Soybean oil.

Another embodiment comprises cyclosporin A, Delonic OPE-12®, and Soybean oil.

Another embodiment comprises cyclosporin A, Delonic OPE-30®, and Soybean oil.

Another embodiment comprises cyclosporin A, Delonic OPE-40®, and Soybean oil.

Another embodiment comprises cyclosporin A, Delonic OPE-5®, and Soybean oil.

Another embodiment comprises cyclosporin A, Delonic OPE-7.5®, and Soybean oil.

Another embodiment comprises cyclosporin A, DePeg 16-CO®, and Soybean oil.

Another embodiment comprises cyclosporin A, DePeg 25-CO®, and Soybean oil.

Another embodiment comprises cyclosporin A, DePeg 30-CO®, and Soybean oil.

Another embodiment comprises cyclosporin A, DePeg 40-CO®, and Soybean oil.

Another embodiment comprises cyclosporin A, DePeg 5-CO®, and Soybean oil.

Another embodiment comprises cyclosporin A, DePeg 80-CO®, and Soybean oil.

Another embodiment comprises cyclosporin A, DeSonic S-100®, and Soybean oil.

Another embodiment comprises cyclosporin A, DeSonic S-114®, and Soybean oil.

Another embodiment comprises cyclosporin A, DeSonic S-405®, and Soybean oil.

Another embodiment comprises cyclosporin A, DeSonic S-45®, and Soybean oil.

Another embodiment comprises cyclosporin A, DeSonic TDA-9®, and Soybean oil.

Another embodiment comprises cyclosporin A, DeThox Acid O-14®, and Soybean oil.

Another embodiment comprises cyclosporin A, DeThox Acid O-16®, and Soybean oil.

Another embodiment comprises cyclosporin A, DeThox Acid O-9®, and Soybean oil.

Another embodiment comprises cyclosporin A, DeThox Acid TO-14®, and Soybean oil.

Another embodiment comprises cyclosporin A, DeThox Acid TO-16.5®, and Soybean oil.

Another embodiment comprises cyclosporin A, DeThox Acid TO-8.5®, and Soybean oil.

Another embodiment comprises cyclosporin A, Dur-Em 114®, and Soybean oil.

Another embodiment comprises cyclosporin A, Dur-Em 117®, and Soybean oil.

Another embodiment comprises cyclosporin A, Dur-Em 204®, and Soybean oil.

Another embodiment comprises cyclosporin A, Dur-Em 207®, and Soybean oil.

Another embodiment comprises cyclosporin A, Dur-Em 207E®, and Soybean oil.

Another embodiment comprises cyclosporin A, Emerest 2704®, and Soybean oil.

Another embodiment comprises cyclosporin A, Emerest 2712®, and Soybean oil.

Another embodiment comprises cyclosporin A, Emerest 2715®, and Soybean oil.

Another embodiment comprises cyclosporin A, Ethal EH-2®, and Soybean oil.

Another embodiment comprises cyclosporin A, Ethal EH-5®, and Soybean oil.

Another embodiment comprises cyclosporin A, Ethal OA-23®, and Soybean oil.

Another embodiment comprises cyclosporin A, Ethosperse CA-20®, and Soybean oil.

Another embodiment comprises cyclosporin A, Ethosperse LA-12®, and Soybean oil.

Another embodiment comprises cyclosporin A, Ethosperse LA-23®, and Soybean oil.

Another embodiment comprises cyclosporin A, Ethosperse LA-4®, and Soybean oil.

Another embodiment comprises cyclosporin A, Ethox 3095®, and Soybean oil.

Another embodiment comprises cyclosporin A, Ethox CO-16®, and Soybean oil.

Another embodiment comprises cyclosporin A, Ethox CO-200®, and Soybean oil.

Another embodiment comprises cyclosporin A, Ethox CO-25®, and Soybean oil.

Another embodiment comprises cyclosporin A, Ethox CO-30®, and Soybean oil.

Another embodiment comprises cyclosporin A, Ethox CO-36®, and Soybean oil.

Another embodiment comprises cyclosporin A, Ethox CO-40®, and Soybean oil.

Another embodiment comprises cyclosporin A, Ethox CO-5®, and Soybean oil.

Another embodiment comprises cyclosporin A, Ethox DO-14®, and Soybean oil.

Another embodiment comprises cyclosporin A, Ethox DO-9®, and Soybean oil.

Another embodiment comprises cyclosporin A, Ethox MA-8®, and Soybean oil.

Another embodiment comprises cyclosporin A, Ethox Ml-14®, and Soybean oil.

Another embodiment comprises cyclosporin A, Ethox ML-14®, and Soybean oil.

Another embodiment comprises cyclosporin A, Ethox ML-5®, and Soybean oil.

Another embodiment comprises cyclosporin A, Ethox MO-14®, and Soybean oil.

Another embodiment comprises cyclosporin A, Ethox MO-9®, and Soybean oil.

Another embodiment comprises cyclosporin A, Ethox MS-14®, and Soybean oil.

Another embodiment comprises cyclosporin A, Ethox MS-23®, and Soybean oil.

Another embodiment comprises cyclosporin A, Ethox MS-40®, and Soybean oil.

Another embodiment comprises cyclosporin A, Ethox MS-8®, and Soybean oil.

Another embodiment comprises cyclosporin A, Ethox TO-16®, and Soybean oil.

Another embodiment comprises cyclosporin A, Ethox TO-8®, and Soybean oil.

Another embodiment comprises cyclosporin A, Ethylan SN-120®, and Soybean oil.

Another embodiment comprises cyclosporin A, Ethylan SN-70®, and Soybean oil.

Another embodiment comprises cyclosporin A, Ethylan SN-90®, and Soybean oil.

Another embodiment comprises cyclosporin A, Flexricin 13®, and Soybean oil.

Another embodiment comprises cyclosporin A, Flexricin 15®, and Soybean oil.

Another embodiment comprises cyclosporin A, Genapol 1261®, and Soybean oil.

Another embodiment comprises cyclosporin A, Generol 122 E25®, and Soybean oil.

Another embodiment comprises cyclosporin A, Glicepol 160®, and Soybean oil.

Another embodiment comprises cyclosporin A, Glicepol 560®, and Soybean oil.

Another embodiment comprises cyclosporin A, Glicepol GMS 20®, and Soybean oil.

Another embodiment comprises cyclosporin A, Hetoxide BN-13®, and Soybean oil.

Another embodiment comprises cyclosporin A, Hetoxide DNP-4®, and Soybean oil.

Another embodiment comprises cyclosporin A, Hetoxide DNP-9.6®, and Soybean oil.

Another embodiment comprises cyclosporin A, Hetoxide G-26®, and Soybean oil.

Another embodiment comprises cyclosporin A, Hetoxide G-7®, and Soybean oil.

Another embodiment comprises cyclosporin A, Hetoxide GT-20®, and Soybean oil.

Another embodiment comprises cyclosporin A, Hetoxide HC-40®, and Soybean oil.

Another embodiment comprises cyclosporin A, Hetoxol C-24®, and Soybean oil.

Another embodiment comprises cyclosporin A, Hetoxol CA-10®, and Soybean oil.

Another embodiment comprises cyclosporin A, Hetoxol CA-2®, and Soybean oil.

Another embodiment comprises cyclosporin A, Hetoxol CA-20®, and Soybean oil.

Another embodiment comprises cyclosporin A, Hetoxol CAWS®, and Soybean oil.

Another embodiment comprises cyclosporin A, Hetoxol CD-3®, and Soybean oil.

Another embodiment comprises cyclosporin A, Hetoxol CD-4®, and Soybean oil.

Another embodiment comprises cyclosporin A, Hetoxol CS-15®, and Soybean oil.

Another embodiment comprises cyclosporin A, Hetoxol CS-20®, and Soybean oil.

Another embodiment comprises cyclosporin A, Hetoxol CS-20D®, and Soybean oil.

Another embodiment comprises cyclosporin A, Hetoxol CS-25®, and Soybean oil.

Another embodiment comprises cyclosporin A, Hetoxol CS-50®, and Soybean oil.

Another embodiment comprises cyclosporin A, Hetoxol CS-9®, and Soybean oil.

Another embodiment comprises cyclosporin A, Hetoxol CSA-15®, and Soybean oil.

Another embodiment comprises cyclosporin A, Hetoxol D®, and Soybean oil.

Another embodiment comprises cyclosporin A, Hetoxol G®, and Soybean oil.

Another embodiment comprises cyclosporin A, Hetoxol J®, and Soybean oil.

Another embodiment comprises cyclosporin A, Hetoxol L®, and Soybean oil.

Another embodiment comprises cyclosporin A, Hetoxol L-23®, and Soybean oil.

Another embodiment comprises cyclosporin A, Hetoxol L-4®, and Soybean oil.

Another embodiment comprises cyclosporin A, Hetoxol L-9®, and Soybean oil.

Another embodiment comprises cyclosporin A, Hetoxol LS-9®, and Soybean oil.

Another embodiment comprises cyclosporin A, Hetoxol M-3®, and Soybean oil.

Another embodiment comprises cyclosporin A, Hetoxol OA-10 Specials®, and Soybean oil.

Another embodiment comprises cyclosporin A, Hetoxol OA-20 Special®, and Soybean oil.

Another embodiment comprises cyclosporin A, Hetoxol OA-3 Special®, and Soybean oil.

Another embodiment comprises cyclosporin A, Hetoxol OA-5 Special®, and Soybean oil.

Another embodiment comprises cyclosporin A, Hetoxol OL-10®, and Soybean oil.

Another embodiment comprises cyclosporin A, Hetoxol OL-2®, and Soybean oil.

Another embodiment comprises cyclosporin A, Hetoxol OL-23®, and Soybean oil.

Another embodiment comprises cyclosporin A, Hetoxol OL-4®, and Soybean oil.

Another embodiment comprises cyclosporin A, Hetoxol OL-40®, and Soybean oil.

Another embodiment comprises cyclosporin A, Hetoxol OL-5®, and Soybean oil.

Another embodiment comprises cyclosporin A, Hetoxol PLA®, and Soybean oil.

Another embodiment comprises cyclosporin A, Hetoxol SP-15®, and Soybean oil.

Another embodiment comprises cyclosporin A, Hetoxol STA-10®, and Soybean oil.

Another embodiment comprises cyclosporin A, Hetoxol STA-2®, and Soybean oil.

Another embodiment comprises cyclosporin A, Hetoxol STA-30®, and Soybean oil.

Another embodiment comprises cyclosporin A, Hetoxol TD-12®, and Soybean oil.

Another embodiment comprises cyclosporin A, Hetoxol TD-18®, and Soybean oil.

Another embodiment comprises cyclosporin A, Hetoxol TD-3®, and Soybean oil.

Another embodiment comprises cyclosporin A, Hetoxol TD-6®, and Soybean oil.

Another embodiment comprises cyclosporin A, Hetoxol TD-9®, and Soybean oil.

Another embodiment comprises cyclosporin A, Hetoxol TDEP-15®, and Soybean oil.

Another embodiment comprises cyclosporin A, Hetoxol TDEP-63®, and Soybean oil.

Another embodiment comprises cyclosporin A, Iconol 24-12®, and Soybean oil.

Another embodiment comprises cyclosporin A, Iconol 24-23®, and Soybean oil.

Another embodiment comprises cyclosporin A, Iconol 24-4®, and Soybean oil.

Another embodiment comprises cyclosporin A, Iconol DA-4®, and Soybean oil.

Another embodiment comprises cyclosporin A, Iconol DA-6®, and Soybean oil.

Another embodiment comprises cyclosporin A, Iconol TDA-10®, and Soybean oil.

Another embodiment comprises cyclosporin A, Iconol TDA-3®, and Soybean oil.

Another embodiment comprises cyclosporin A, Iconol TDA-6®, and Soybean oil.

Another embodiment comprises cyclosporin A, Iconol TDA-8-90%®, and Soybean oil.

Another embodiment comprises cyclosporin A, Iconol TDA-9®, and Soybean oil.

Another embodiment comprises cyclosporin A, Igepal CA-210®, and Soybean oil.

Another embodiment comprises cyclosporin A, Igepal CA-407®, and Soybean oil.

Another embodiment comprises cyclosporin A, Igepal CA-420®, and Soybean oil.

Another embodiment comprises cyclosporin A, Igepal CA-520®, and Soybean oil.

Another embodiment comprises cyclosporin A, Igepal CA-620®, and Soybean oil.

Another embodiment comprises cyclosporin A, Igepal CA-630®, and Soybean oil.

Another embodiment comprises cyclosporin A, Igepal CA-720®, and Soybean oil.

Another embodiment comprises cyclosporin A, Igepal CA-877®, and Soybean oil.

Another embodiment comprises cyclosporin A, Igepal CA-887®, and Soybean oil.

Another embodiment comprises cyclosporin A, Igepal CA-897®, and Soybean oil.

Another embodiment comprises cyclosporin A, Igepal CO-210®, and Soybean oil.

Another embodiment comprises cyclosporin A, Igepal CO-430®, and Soybean oil.

Another embodiment comprises cyclosporin A, Igepal CO-520®, and Soybean oil.

Another embodiment comprises cyclosporin A, Igepal CO-530®, and Soybean oil.

Another embodiment comprises cyclosporin A, Igepal CO-610®, and Soybean oil.

Another embodiment comprises cyclosporin A, Igepal CO-630®, and Soybean oil.

Another embodiment comprises cyclosporin A, Igepal CO-630 Special®, and Soybean oil.

Another embodiment comprises cyclosporin A, Igepal CO-660®, and Soybean oil.

Another embodiment comprises cyclosporin A, Igepal CO-710®, and Soybean oil.

Another embodiment comprises cyclosporin A, Igepal CO-720®, and Soybean oil.

Another embodiment comprises cyclosporin A, Igepal CO-730®, and Soybean oil.

Another embodiment comprises cyclosporin A, Igepal CO-738®, and Soybean oil.

Another embodiment comprises cyclosporin A, Igepal CO-850®, and Soybean oil.

Another embodiment comprises cyclosporin A, Igepal CO-880®, and Soybean oil.

Another embodiment comprises cyclosporin A, Igepal CO-887®, and Soybean oil.

Another embodiment comprises cyclosporin A, Igepal CO-890®, and Soybean oil.

Another embodiment comprises cyclosporin A, Igepal CO-897®, and Soybean oil.

Another embodiment comprises cyclosporin A, Igepal CO-970®, and Soybean oil.

Another embodiment comprises cyclosporin A, Igepal CO-977®, and Soybean oil.

Another embodiment comprises cyclosporin A, Igepal CO-987®, and Soybean oil.

Another embodiment comprises cyclosporin A, Igepal CO-990 FLK®, and Soybean oil.

Another embodiment comprises cyclosporin A, Igepal CO-997®, and Soybean oil.

Another embodiment comprises cyclosporin A, Igepal DM-430®, and Soybean oil.

Another embodiment comprises cyclosporin A, Igepal DM-530®, and Soybean oil.

Another embodiment comprises cyclosporin A, Igepal DM-710®, and Soybean oil.

Another embodiment comprises cyclosporin A, Igepal DM-730®, and Soybean oil.

Another embodiment comprises cyclosporin A, Igepal DM-880®, and Soybean oil.

Another embodiment comprises cyclosporin A, Igepal DM-970 FLK®, and Soybean oil.

Another embodiment comprises cyclosporin A, Igepal OD410®, and Soybean oil.

Another embodiment comprises cyclosporin A, Igepal RC-520®, and Soybean oil.

Another embodiment comprises cyclosporin A, Igepal RC-620®, and Soybean oil.

Another embodiment comprises cyclosporin A, Igepal RC-630®, and Soybean oil.

Another embodiment comprises cyclosporin A, Lexemul CS-20®, and Soybean oil.

Another embodiment comprises cyclosporin A, Lipo GMS 450®, and Soybean oil.

Another embodiment comprises cyclosporin A, Lipo Polyglycol 1000®, and Soybean oil.

Another embodiment comprises cyclosporin A, Lipo Polyglycol 200®, and Soybean oil.

Another embodiment comprises cyclosporin A, Lipo Polyglycol 300®, and Soybean oil.

Another embodiment comprises cyclosporin A, Lipo Polyglycol 3350®, and Soybean oil.

Another embodiment comprises cyclosporin A, Lipo Polyglycol 400®, and Soybean oil.

Another embodiment comprises cyclosporin A, Lipo Polyglycol 600®, and Soybean oil.

Another embodiment comprises cyclosporin A, Lipocol C-10®, and Soybean oil.

Another embodiment comprises cyclosporin A, Lipocol C-2®, and Soybean oil.

Another embodiment comprises cyclosporin A, Lipocol C-20®, and Soybean oil.

Another embodiment comprises cyclosporin A, Lipocol L-12®, and Soybean oil.

Another embodiment comprises cyclosporin A, Lipocol L-23®, and Soybean oil.

Another embodiment comprises cyclosporin A, Lipocol L-4®, and Soybean oil.

Another embodiment comprises cyclosporin A, Lipocol O-10®, and Soybean oil.

Another embodiment comprises cyclosporin A, Lipocol O-2®, and Soybean oil.

Another embodiment comprises cyclosporin A, Lipocol O-20®, and Soybean oil.

Another embodiment comprises cyclosporin A, Lipocol O-5®, and Soybean oil.

Another embodiment comprises cyclosporin A, Lipocol S-10®, and Soybean oil.

Another embodiment comprises cyclosporin A, Lipocol S-2®, and Soybean oil.

Another embodiment comprises cyclosporin A, Lipocol S-20®, and Soybean oil.

Another embodiment comprises cyclosporin A, Lipocol SC-15®, and Soybean oil.

Another embodiment comprises cyclosporin A, Lipocol SC-20®, and Soybean oil.

Another embodiment comprises cyclosporin A, Lipocol SC-4®, and Soybean oil.

Another embodiment comprises cyclosporin A, Lipocol TD-12®, and Soybean oil.

Another embodiment comprises cyclosporin A, Lumulse 40-O K®, and Soybean oil.

Another embodiment comprises cyclosporin A, Lumulse 42-OK®, and Soybean oil.

Another embodiment comprises cyclosporin A, Lumulse GMT-40®, and Soybean oil.

Another embodiment comprises cyclosporin A, Lumulse PEG 1450®, and Soybean oil.

Another embodiment comprises cyclosporin A, Lumulse PEG 1450 NF®, and Soybean oil.

Another embodiment comprises cyclosporin A, Lumulse PEG 8000®, and Soybean oil.

Another embodiment comprises cyclosporin A, Lumulse PGO®, and Soybean oil.

Another embodiment comprises cyclosporin A, Lumulse POE (2) Oleyl Amine®, and Soybean oil.

Another embodiment comprises cyclosporin A, Magrabar PGO-104®, and Soybean oil.

Another embodiment comprises cyclosporin A, Mapeg 200 ML®, and Soybean oil.

Another embodiment comprises cyclosporin A, Mapeg 400 MO®, and Soybean oil.

Another embodiment comprises cyclosporin A, Mapeg 400 MOT®, and Soybean oil.

Another embodiment comprises cyclosporin A, Mapeg 400DO®, and Soybean oil.

Another embodiment comprises cyclosporin A, Mapeg 400DOT®, and Soybean oil.

Another embodiment comprises cyclosporin A, Mapeg 600DOT®, and Soybean oil.

Another embodiment comprises cyclosporin A, Marlox B 24/80®, and Soybean oil.

Another embodiment comprises cyclosporin A, Marlox FK 86®, and Soybean oil.

Another embodiment comprises cyclosporin A, Marlox MO 154®, and Soybean oil.

Another embodiment comprises cyclosporin A, Mazox CDA CG®, and Soybean oil.

Another embodiment comprises cyclosporin A, Monoglycerides Glyceryl Monestearate®, and Soybean oil.

Another embodiment comprises cyclosporin A, Myrj 45®, and Soybean oil.

Another embodiment comprises cyclosporin A, Myrj 52®, and Soybean oil.

Another embodiment comprises cyclosporin A, Myrj 52S®, and Soybean oil.

Another embodiment comprises cyclosporin A, Myrj 53®, and Soybean oil.

Another embodiment comprises cyclosporin A, Myrj 59®, and Soybean oil.

Another embodiment comprises cyclosporin A, Nortox NP-12®, and Soybean oil.

Another embodiment comprises cyclosporin A, Nortox NP-4®, and Soybean oil.

Another embodiment comprises cyclosporin A, Norfox NP-6®, and Soybean oil.

Another embodiment comprises cyclosporin A, Norfox NP-887®, and Soybean oil.

Another embodiment comprises cyclosporin A, Norfox NP-9®, and Soybean oil.

Another embodiment comprises cyclosporin A, Norfox NP-977®, and Soybean oil.

Another embodiment comprises cyclosporin A, Norfox OP-100®, and Soybean oil.

Another embodiment comprises cyclosporin A, Norfox OP-102®, and Soybean oil.

Another embodiment comprises cyclosporin A, Norfox OP-114®, and Soybean oil.

Another embodiment comprises cyclosporin A, Norfox OP-45®, and Soybean oil.

Another embodiment comprises cyclosporin A, Pegosperse 100-L®, and Soybean oil.

Another embodiment comprises cyclosporin A, Pegosperse 100-O®, and Soybean oil.

Another embodiment comprises cyclosporin A, Pegosperse 100-S®, and Soybean oil.

Another embodiment comprises cyclosporin A, Pegosperse 1500-MS®, and Soybean oil.

Another embodiment comprises cyclosporin A, Pegosperse 1750-MS®, and Soybean oil.

Another embodiment comprises cyclosporin A, Pegosperse 200 DL®, and Soybean oil.

Another embodiment comprises cyclosporin A, Pegosperse 200-ML®, and Soybean oil.

Another embodiment comprises cyclosporin A, Pegosperse 400-DL®, and Soybean oil.

Another embodiment comprises cyclosporin A, Pegosperse 400-DO®, and Soybean oil.

Another embodiment comprises cyclosporin A, Pegosperse 400-DS®, and Soybean oil.

Another embodiment comprises cyclosporin A, Pegosperse 400-ML®, and Soybean oil.

Another embodiment comprises cyclosporin A, Pegosperse 400-MO®, and Soybean oil.

Another embodiment comprises cyclosporin A, Pegosperse 400-MOT®, and Soybean oil.

Another embodiment comprises cyclosporin A, Pegosperse 400-MS®, and Soybean oil.

Another embodiment comprises cyclosporin A, Pegosperse 50-DS®, and Soybean oil.

Another embodiment comprises cyclosporin A, Pegosperse 50-MS®, and Soybean oil.

Another embodiment comprises cyclosporin A, Pegosperse 600-ML®, and Soybean oil.

Another embodiment comprises cyclosporin A, Pegosperse 600-MS®, and Soybean oil.

Another embodiment comprises cyclosporin A, Plurafac A-38®, and Soybean oil.

Another embodiment comprises cyclosporin A, Plurafac B-25-5®, and Soybean oil.

Another embodiment comprises cyclosporin A, Plurafac B-26®, and Soybean oil.

Another embodiment comprises cyclosporin A, Plurafac D-25®, and Soybean oil.

Another embodiment comprises cyclosporin A, Plurafac LF 131®, and Soybean oil.

Another embodiment comprises cyclosporin A, Plurafac LF 4030®, and Soybean oil.

Another embodiment comprises cyclosporin A, Plurafac LF 7000®, and Soybean oil.

Another embodiment comprises cyclosporin A, Plurafac RA-20®, and Soybean oil.

Another embodiment comprises cyclosporin A, Plurafac RA-30®, and Soybean oil.

Another embodiment comprises cyclosporin A, Plurafac RA-40®, and Soybean oil.

Another embodiment comprises cyclosporin A, Pluronic 10R5®, and Soybean oil.

Another embodiment comprises cyclosporin A, Pluronic 17R23®, and Soybean oil.

Another embodiment comprises cyclosporin A, Pluronic 17R4®, and Soybean oil.

Another embodiment comprises cyclosporin A, Pluronic 25R2®, and Soybean oil.

Another embodiment comprises cyclosporin A, Pluronic 25R4®, and Soybean oil.

Another embodiment comprises cyclosporin A, Pluronic 31R1®, and Soybean oil.

Another embodiment comprises cyclosporin A, Pluronic F108®, and Soybean oil.

Another embodiment comprises cyclosporin A, Pluronic F127®, and Soybean oil.

Another embodiment comprises cyclosporin A, Pluronic F38®, and Soybean oil.

Another embodiment comprises cyclosporin A, Pluronic F68®, and Soybean oil.

Another embodiment comprises cyclosporin A, Pluronic F68LF®, and Soybean oil.

Another embodiment comprises cyclosporin A, Pluronic F77®, and Soybean oil.

Another embodiment comprises cyclosporin A, Pluronic F87®, and Soybean oil.

Another embodiment comprises cyclosporin A, Pluronic F88®, and Soybean oil.

Another embodiment comprises cyclosporin A, Pluronic F98®, and Soybean oil.

Another embodiment comprises cyclosporin A, Pluronic L10®, and Soybean oil.

Another embodiment comprises cyclosporin A, Pluronic L101®, and Soybean oil.

Another embodiment comprises cyclosporin A, Pluronic L121®, and Soybean oil.

Another embodiment comprises cyclosporin A, Pluronic L31®, and Soybean oil.

Another embodiment comprises cyclosporin A, Pluronic L35®, and Soybean oil.

Another embodiment comprises cyclosporin A, Pluronic L43®, and Soybean oil.

Another embodiment comprises cyclosporin A, Pluronic L44®, and Soybean oil.

Another embodiment comprises cyclosporin A, Pluronic L61®, and Soybean oil.

Another embodiment comprises cyclosporin A, Pluronic L62®, and Soybean oil.

Another embodiment comprises cyclosporin A, Pluronic L62D®, and Soybean oil.

Another embodiment comprises cyclosporin A, Pluronic L62LF®, and Soybean oil.

Another embodiment comprises cyclosporin A, Pluronic L64®, and Soybean oil.

Another embodiment comprises cyclosporin A, Pluronic L81®, and Soybean oil.

Another embodiment comprises cyclosporin A, Pluronic L92®, and Soybean oil.

Another embodiment comprises cyclosporin A, Pluronic P103®, and Soybean oil.

Another embodiment comprises cyclosporin A, Pluronic P104®, and Soybean oil.

Another embodiment comprises cyclosporin A, Pluronic P105®, and Soybean oil.

Another embodiment comprises cyclosporin A, Pluronic P123®, and Soybean oil.

Another embodiment comprises cyclosporin A, Pluronic P65®, and Soybean oil.

Another embodiment comprises cyclosporin A, Pluronic P84®, and Soybean oil.

Another embodiment comprises cyclosporin A, Pluronic P85®, and Soybean oil.

Another embodiment comprises cyclosporin A, Polyaldo 10-2-P®, and Soybean oil.

Another embodiment comprises cyclosporin A, Polyaldo DGDO®, and Soybean oil.

Another embodiment comprises cyclosporin A, Polyaldo HGDS®, and Soybean oil.

Another embodiment comprises cyclosporin A, Polyaldo TGMS®, and Soybean oil.

Another embodiment comprises cyclosporin A, Protegin V®, and Soybean oil.

Another embodiment comprises cyclosporin A, Protegin W®, and Soybean oil.

Another embodiment comprises cyclosporin A, Protegin WX®, and Soybean oil.

Another embodiment comprises cyclosporin A, Protegin XV®, and Soybean oil.

Another embodiment comprises cyclosporin A, Rhodasurf BC-420®, and Soybean oil.

Another embodiment comprises cyclosporin A, Rhodasurf BC-610®, and Soybean oil.

Another embodiment comprises cyclosporin A, Rhodasurf BC-630®, and Soybean oil.

Another embodiment comprises cyclosporin A, Rhodasurf BC-720®, and Soybean oil.

Another embodiment comprises cyclosporin A, Rhodasurf BC-840®, and Soybean oil.

Another embodiment comprises cyclosporin A, Rhodasurf L 1®, and Soybean oil.

Another embodiment comprises cyclosporin A, Rhodasurf L 12®, and Soybean oil.

Another embodiment comprises cyclosporin A, Rhodasurf L 2®, and Soybean oil.

Another embodiment comprises cyclosporin A, Rhodasurf L 20®, and Soybean oil.

Another embodiment comprises cyclosporin A, Rhodasurf L 3®, and Soybean oil.

Another embodiment comprises cyclosporin A, Rhodasun L 30®, and Soybean oil.

Another embodiment comprises cyclosporin A, Rhodasurf L 4®, and Soybean oil.

Another embodiment comprises cyclosporin A, Rhodasurf L 50®, and Soybean oil.

Another embodiment comprises cyclosporin A, Rhodasurf L 7/90®, and Soybean oil.

Another embodiment comprises cyclosporin A, Rhodasurf LA-3®, and Soybean oil.

Another embodiment comprises cyclosporin A, Rhodasurf LA-7®, and Soybean oil.

Another embodiment comprises cyclosporin A, Rhodasurf LAN-23®, and Soybean oil.

Another embodiment comprises cyclosporin A, Rhodasurf ON-870®, and Soybean oil.

Another embodiment comprises cyclosporin A, Rhodasun ON-877®, and Soybean oil.

Another embodiment comprises cyclosporin A, Rhodasun TDA-8/5®, and Soybean oil.

Another embodiment comprises cyclosporin A, Ritapro 100®, and Soybean oil.

Another embodiment comprises cyclosporin A, Ritapro 165®, and Soybean oil.

Another embodiment comprises cyclosporin A, Ritapro 200®, and Soybean oil.

Another embodiment comprises cyclosporin A, Ritapro 300®, and Soybean oil.

Another embodiment comprises cyclosporin A, Ritoleth 10®, and Soybean oil.

Another embodiment comprises cyclosporin A, Ritoleth 2®, and Soybean oil.

Another embodiment comprises cyclosporin A, Ritoleth 20®, and Soybean oil.

Another embodiment comprises cyclosporin A, Ritoleth 5®, and Soybean oil.

Another embodiment comprises cyclosporin A, Santone 10-10-0®, and Soybean oil.

Another embodiment comprises cyclosporin A, Santone 3-1-S XTR®, and Soybean oil.

Another embodiment comprises cyclosporin A, Santone 3-1-SH®, and Soybean oil.

Another embodiment comprises cyclosporin A, Santone 8-1-O®, and Soybean oil.

Another embodiment comprises cyclosporin A, Solulan 16®, and Soybean oil.

Another embodiment comprises cyclosporin A, Solulan 75®, and Soybean oil.

Another embodiment comprises cyclosporin A, Solulan C-24®, and Soybean oil.

Another embodiment comprises cyclosporin A, Solulan L-575®, and Soybean oil.

Another embodiment comprises cyclosporin A, Standamox CAW®, and Soybean oil.

Another embodiment comprises cyclosporin A, Surtine AZI-A®, and Soybean oil.

Another embodiment comprises cyclosporin A, Surtonic CO-15®, and Soybean oil.

Another embodiment comprises cyclosporin A, Surtonic CO-25®, and Soybean oil.

Another embodiment comprises cyclosporin A, Sudonic CO-30®, and Soybean oil.

Another embodiment comprises cyclosporin A, Surtonic CO-36®, and Soybean oil.

Another embodiment comprises cyclosporin A, Surfonic CO-42®, and Soybean oil.

Another embodiment comprises cyclosporin A, Surfonic E400-MO®, and Soybean oil.

Another embodiment comprises cyclosporin A, Surfonic L12-3®, and Soybean oil.

Another embodiment comprises cyclosporin A, Surfonic L12-6®, and Soybean oil.

Another embodiment comprises cyclosporin A, Surfonic L12-8®, and Soybean oil.

Another embodiment comprises cyclosporin A, Surfonic L24-12®, and Soybean oil.

Another embodiment comprises cyclosporin A, Surfonic L24-2®, and Soybean oil.

Another embodiment comprises cyclosporin A, Surfonic L24-22®, and Soybean oil.

Another embodiment comprises cyclosporin A, Surfonic L24-3®, and Soybean oil.

Another embodiment comprises cyclosporin A, Surfonic L24-4®, and Soybean oil.

Another embodiment comprises cyclosporin A, Surfonic L24-5®, and Soybean oil.

Another embodiment comprises cyclosporin A, Surfonic L24-7®, and Soybean oil.

Another embodiment comprises cyclosporin A, Surfonic L24-7.1®, and Soybean oil.

Another embodiment comprises cyclosporin A, Surfonic L24-9®, and Soybean oil.

Another embodiment comprises cyclosporin A, Surfonic LF-17®, and Soybean oil.

Another embodiment comprises cyclosporin A, Surfonic LF-18®, and Soybean oil.

Another embodiment comprises cyclosporin A, Surfonic LF-37®, and Soybean oil.

Another embodiment comprises cyclosporin A, Surfonic LF-41®, and Soybean oil.

Another embodiment comprises cyclosporin A, Surfonic LF-42®, and Soybean oil.

Another embodiment comprises cyclosporin A, Surfonic LF-47®, and Soybean oil.

Another embodiment comprises cyclosporin A, Surfonic LF-68®, and Soybean oil.

Another embodiment comprises cyclosporin A, Surfonic N-10®, and Soybean oil.

Another embodiment comprises cyclosporin A, Surfonic N-100®, and Soybean oil.

Another embodiment comprises cyclosporin A, Surfonic N-102®, and Soybean oil.

Another embodiment comprises cyclosporin A, Surfonic N-120®, and Soybean oil.

Another embodiment comprises cyclosporin A, Surfonic N-150®, and Soybean oil.

Another embodiment comprises cyclosporin A, Surfonic N-200®, and Soybean oil.

Another embodiment comprises cyclosporin A, Surfonic N-300®, and Soybean oil.

Another embodiment comprises cyclosporin A, Surfonic N-31.5®, and Soybean oil.

Another embodiment comprises cyclosporin A, Surfonic N-40®, and Soybean oil.

Another embodiment comprises cyclosporin A, Surfonic N-400®, and Soybean oil.

Another embodiment comprises cyclosporin A, Surfonic N-60®, and Soybean oil.

Another embodiment comprises cyclosporin A, Surfonic N-85®, and Soybean oil.

Another embodiment comprises cyclosporin A, Surfonic N-95®, and Soybean oil.

Another embodiment comprises cyclosporin A, Surfonic NB-18®, and Soybean oil.

Another embodiment comprises cyclosporin A, Surfonic NB-23®, and Soybean oil.

Another embodiment comprises cyclosporin A, Surfonic NB-25®, and Soybean oil.

Another embodiment comprises cyclosporin A, Surfonic NB-307®, and Soybean oil.

Another embodiment comprises cyclosporin A, Surfonic NB-31®, and Soybean oil.

Another embodiment comprises cyclosporin A, Surfonic NB-41®, and Soybean oil.

Another embodiment comprises cyclosporin A, Surfonic NB-43®, and Soybean oil.

Another embodiment comprises cyclosporin A, Surfonic OP-100®, and Soybean oil.

Another embodiment comprises cyclosporin A, Surfonic OP-120®, and Soybean oil.

Another embodiment comprises cyclosporin A, Surfonic OP-15®, and Soybean oil.

Another embodiment comprises cyclosporin A, Surfonic OP-35®, and Soybean oil.

Another embodiment comprises cyclosporin A, Surfonic OP-70®, and Soybean oil.

Another embodiment comprises cyclosporin A, Surfonic P1®, and Soybean oil.

Another embodiment comprises cyclosporin A, Surfonic P3®, and Soybean oil.

Another embodiment comprises cyclosporin A, Surfonic P5®, and Soybean oil.

Another embodiment comprises cyclosporin A, Surfonic P6®, and Soybean oil.

Another embodiment comprises cyclosporin A, Surfonic POA-17R2®, and Soybean oil.

Another embodiment comprises cyclosporin A, Surfonic POA-25R2®, and Soybean oil.

Another embodiment comprises cyclosporin A, Surfonic TDA-11®, and Soybean oil.

Another embodiment comprises cyclosporin A, Surfonic TDA-3B®, and Soybean oil.

Another embodiment comprises cyclosporin A, Surfonic TDA-6®, and Soybean oil.

Another embodiment comprises cyclosporin A, Surfonic TDA-6/88®, and Soybean oil.

Another embodiment comprises cyclosporin A, Surfonic TDA-8®, and Soybean oil.

Another embodiment comprises cyclosporin A, Surfonic TDA-8/90®, and Soybean oil.

Another embodiment comprises cyclosporin A, Surfonic TDA-9®, and Soybean oil.

Another embodiment comprises cyclosporin A, Surfonic TX-CCN®, and Soybean oil.

Another embodiment comprises cyclosporin A, Tagat CH 40®, and Soybean oil.

Another embodiment comprises cyclosporin A, Tagat CH 60®, and Soybean oil.

Another embodiment comprises cyclosporin A, Tagat L 2®, and Soybean oil.

Another embodiment comprises cyclosporin A, Tagat S®, and Soybean oil.

Another embodiment comprises cyclosporin A, Tagat S 2®, and Soybean oil.

Another embodiment comprises cyclosporin A, Tally 100 Plus®, and Soybean oil.

Another embodiment comprises cyclosporin A, T-Det A106®, and Soybean oil.

Another embodiment comprises cyclosporin A, T-Det A109®, and Soybean oil.

Another embodiment comprises cyclosporin A, T-Det A136®, and Soybean oil.

Another embodiment comprises cyclosporin A, T-Det A139®, and Soybean oil.

Another embodiment comprises cyclosporin A, T-Det A2412®, and Soybean oil.

Another embodiment comprises cyclosporin A, T-Det A243®, and Soybean oil.

Another embodiment comprises cyclosporin A, T-Det A247®, and Soybean oil.

Another embodiment comprises cyclosporin A, T-Det A249®, and Soybean oil.

Another embodiment comprises cyclosporin A, T-Det A467®, and Soybean oil.

Another embodiment comprises cyclosporin A, T-Det COE®, and Soybean oil.

Another embodiment comprises cyclosporin A, T-Det DD-10®, and Soybean oil.

Another embodiment comprises cyclosporin A, T-Det DD-5®, and Soybean oil.

Another embodiment comprises cyclosporin A, T-Det DD-7®, and Soybean oil.

Another embodiment comprises cyclosporin A, T-Det EPO-104®, and Soybean oil.

Another embodiment comprises cyclosporin A, T-Det EPO-61®, and Soybean oil.

Another embodiment comprises cyclosporin A, T-Det EPO-62®, and Soybean oil.

Another embodiment comprises cyclosporin A, T-Det EPO-64®, and Soybean oil.

Another embodiment comprises cyclosporin A, T-Det N-1.5®, and Soybean oil.

Another embodiment comprises cyclosporin A, T-Det N-10.5®, and Soybean oil.

Another embodiment comprises cyclosporin A, T-Det N-100®, and Soybean oil.

Another embodiment comprises cyclosporin A, T-Det N-1007®, and Soybean oil.

Another embodiment comprises cyclosporin A, T-Det N-12®, and Soybean oil.

Another embodiment comprises cyclosporin A, T-Det N-14®, and Soybean oil.

Another embodiment comprises cyclosporin A, T-Det N-20®, and Soybean oil.

Another embodiment comprises cyclosporin A, T-Det N-30®, and Soybean oil.

Another embodiment comprises cyclosporin A, T-Det N-307®, and Soybean oil.

Another embodiment comprises cyclosporin A, T-Det N-4®, and Soybean oil.

Another embodiment comprises cyclosporin A, T-Det N-40®, and Soybean oil.

Another embodiment comprises cyclosporin A, T-Det N-407®, and Soybean oil.

Another embodiment comprises cyclosporin A, T-Det N-50®, and Soybean oil.

Another embodiment comprises cyclosporin A, T-Det N-507®, and Soybean oil.

Another embodiment comprises cyclosporin A, T-Det N-6®, and Soybean oil.

Another embodiment comprises cyclosporin A, T-Det N-70®, and Soybean oil.

Another embodiment comprises cyclosporin A, T-Det N-8®, and Soybean oil.

Another embodiment comprises cyclosporin A, T-Det N-9.5®, and Soybean oil.

Another embodiment comprises cyclosporin A, T-Det O-12®, and Soybean oil.

Another embodiment comprises cyclosporin A, T-Det O-165®, and Soybean oil.

Another embodiment comprises cyclosporin A, T-Det O-307®, and Soybean oil.

Another embodiment comprises cyclosporin A, T-Det O-40®, and Soybean oil.

Another embodiment comprises cyclosporin A, T-Det O-407®, and Soybean oil.

Another embodiment comprises cyclosporin A, T-Det O-6®, and Soybean oil.

Another embodiment comprises cyclosporin A, T-Det O-8®, and Soybean oil.

Another embodiment comprises cyclosporin A, T-Det O-9®, and Soybean oil.

Another embodiment comprises cyclosporin A, Tegin®, and Soybean oil.

Another embodiment comprises cyclosporin A, Tergitol 15-S-12®, and Soybean oil.

Another embodiment comprises cyclosporin A, Tergitol 15-S-15®, and Soybean oil.

Another embodiment comprises cyclosporin A, Tergitol 15-S-20®, and Soybean oil.

Another embodiment comprises cyclosporin A, Tergitol 15-S-20 (80AQ)®, and Soybean oil.

Another embodiment comprises cyclosporin A, Tergitol 15-S-3®, and Soybean oil.

Another embodiment comprises cyclosporin A, Tergitol 15-S-30®, and Soybean oil.

Another embodiment comprises cyclosporin A, Tergitol 15-S-40®, and Soybean oil.

Another embodiment comprises cyclosporin A, Tergitol 15-S-40 (70AQ)®, and Soybean oil.

Another embodiment comprises cyclosporin A, Tergitol 15-S-5®, and Soybean oil.

Another embodiment comprises cyclosporin A, Tergitol 15-S-7®, and Soybean oil.

Another embodiment comprises cyclosporin A, Tergitol 15-S-9®, and Soybean oil.

Another embodiment comprises cyclosporin A, Tergitol L-101®, and Soybean oil.

Another embodiment comprises cyclosporin A, Tergitol L-61®, and Soybean oil.

Another embodiment comprises cyclosporin A, Tergitol L-62®, and Soybean oil.

Another embodiment comprises cyclosporin A, Tergitol L-64®, and Soybean oil.

Another embodiment comprises cyclosporin A, Tergitol L-81®, and Soybean oil.

Another embodiment comprises cyclosporin A, Tergitol NP-10®, and Soybean oil.

Another embodiment comprises cyclosporin A, Tergitol NP-11®, and Soybean oil.

Another embodiment comprises cyclosporin A, Tergitol NP-12®, and Soybean oil.

Another embodiment comprises cyclosporin A, Tergitol NP-13®, and Soybean oil.

Another embodiment comprises cyclosporin A, Tergitol NP-15®, and Soybean oil.

Another embodiment comprises cyclosporin A, Tergitol NP-30®, and Soybean oil.

Another embodiment comprises cyclosporin A, Tergitol NP-30 (70 AQ)®, and Soybean oil.

Another embodiment comprises cyclosporin A, Tergitol NP-4®, and Soybean oil.

Another embodiment comprises cyclosporin A, Tergitol NP-40®, and Soybean oil.

Another embodiment comprises cyclosporin A, Tergitol NP-40 (70 AQ)®, and Soybean oil.

Another embodiment comprises cyclosporin A, Tergitol NP-50 (70 AQ)®, and Soybean oil.

Another embodiment comprises cyclosporin A, Tergitol NP-55 (70 AQ)®, and Soybean oil.

Another embodiment comprises cyclosporin A, Tergitol NP-6®, and Soybean oil.

Another embodiment comprises cyclosporin A, Tergitol NP-7®, and Soybean oil.

Another embodiment comprises cyclosporin A, Tergitol NP-70 (70 AQ)®, and Soybean oil.

Another embodiment comprises cyclosporin A, Tergitol NP-8®, and Soybean oil.

Another embodiment comprises cyclosporin A, Tergitol NP-9®, and Soybean oil.

Another embodiment comprises cyclosporin A, Tergitol NP-9.5®, and Soybean oil.

Another embodiment comprises cyclosporin A, Tergitol TMN-10®, and Soybean oil.

Another embodiment comprises cyclosporin A, Tergitol TMN-3®, and Soybean oil.

Another embodiment comprises cyclosporin A, Tergitol TMN-6®, and Soybean oil.

Another embodiment comprises cyclosporin A, Tergitol XD®, and Soybean oil.

Another embodiment comprises cyclosporin A, Tergitol XH®, and Soybean oil.

Another embodiment comprises cyclosporin A, Tergitol XJ®, and Soybean oil.

Another embodiment comprises cyclosporin A, Tetronic 1107®, and Soybean oil.

Another embodiment comprises cyclosporin A, Tetronic 1307®, and Soybean oil.

Another embodiment comprises cyclosporin A, Tetronic 150R1®, and Soybean oil.

Another embodiment comprises cyclosporin A, Tetronic 304®, and Soybean oil.

Another embodiment comprises cyclosporin A, Tetronic 701®, and Soybean oil.

Another embodiment comprises cyclosporin A, Tetronic 901®, and Soybean oil.

Another embodiment comprises cyclosporin A, Tetronic 904®, and Soybean oil.

Another embodiment comprises cyclosporin A, Tetronic 908®, and Soybean oil.

Another embodiment comprises cyclosporin A, Tetronic 90R4®, and Soybean oil.

Another embodiment comprises cyclosporin A, Tomadol 1-3®, and Soybean oil.

Another embodiment comprises cyclosporin A, Tomadol 1-5®, and Soybean oil.

Another embodiment comprises cyclosporin A, Tomadol 1-7®, and Soybean oil.

Another embodiment comprises cyclosporin A, Tomadol 23-1®, and Soybean oil.

Another embodiment comprises cyclosporin A, Tomadol 23-3®, and Soybean oil.

Another embodiment comprises cyclosporin A, Tomadol 23-5®, and Soybean oil.

Another embodiment comprises cyclosporin A, Tomadol 23-6.5®, and Soybean oil.

Another embodiment comprises cyclosporin A, Tomadol 25-12®, and Soybean oil.

Another embodiment comprises cyclosporin A, Tomadol 25-3®, and Soybean oil.

Another embodiment comprises cyclosporin A, Tomadol 25-7®, and Soybean oil.

Another embodiment comprises cyclosporin A, Tomadol 25-9®, and Soybean oil.

Another embodiment comprises cyclosporin A, Tomadol 45-13®, and Soybean oil.

Another embodiment comprises cyclosporin A, Tomadol 45-2.5®, and Soybean oil.

Another embodiment comprises cyclosporin A, Tomadol 45-7®, and Soybean oil.

Another embodiment comprises cyclosporin A, Tomadol 91-2.5®, and Soybean oil.

Another embodiment comprises cyclosporin A, Tomadol 91-6®, and Soybean oil.

Another embodiment comprises cyclosporin A, Tomadol 91-8®, and Soybean oil.

Another embodiment comprises cyclosporin A, Triton X-100®, and Soybean oil.

Another embodiment comprises cyclosporin A, Triton X-102®,and Soybean oil.

Another embodiment comprises cyclosporin A, Triton X-114®, and Soybean oil.

Another embodiment comprises cyclosporin A, Triton X-15®, and Soybean oil.

Another embodiment comprises cyclosporin A, Triton X-165 (70%)®, and Soybean oil.

Another embodiment comprises cyclosporin A, Triton X-207®, and Soybean oil.

Another embodiment comprises cyclosporin A, Triton X-305 (70%)®, and Soybean oil.

Another embodiment comprises cyclosporin A, Triton X-35®, and Soybean oil.

Another embodiment comprises cyclosporin A, Triton X-405 (70%)®, and Soybean oil.

Another embodiment comprises cyclosporin A, Triton X-45®, and Soybean oil.

Another embodiment comprises cyclosporin A, Triton X-705 (70%)®, and Soybean oil.

Another embodiment comprises cyclosporin A, Volpo S-10®, and Soybean oil.

Another embodiment comprises cyclosporin A, Volpo S-2®, and Soybean oil.

Another embodiment comprises cyclosporin A, Volpo S-20®, and Soybean oil.

Another embodiment comprises cyclosporin A, Volpo-10®, and Soybean oil.

Another embodiment comprises cyclosporin A, Volpo-20®, and Soybean oil.

Another embodiment comprises cyclosporin A, Volpo-3®, and Soybean oil.

Another embodiment comprises cyclosporin A, Volpo-5®, and Soybean oil.

Another embodiment comprises cyclosporin A, Abex 2515®, and Olive oil.

Another embodiment comprises cyclosporin A, Abex 2525/40®, and Olive oil.

Another embodiment comprises cyclosporin A, Abex 2535®, and Olive oil.

Another embodiment comprises cyclosporin A, Abex 2545®, and Olive oil.

Another embodiment comprises cyclosporin A, Acconon E®, and Olive oil.

Another embodiment comprises cyclosporin A, Alfonic 1216CO-9®, and Olive oil.

Another embodiment comprises cyclosporin A, Alfonic 1412-3®, and Olive oil.

Another embodiment comprises cyclosporin A, Alfonic TDA-12®, and Olive oil.

Another embodiment comprises cyclosporin A, Alfonic TDA-3®, and Olive oil.

Another embodiment comprises cyclosporin A, Alfonic TDA-4®, and Olive oil.

Another embodiment comprises cyclosporin A, Alfonic TDA-6®, and Olive oil.

Another embodiment comprises cyclosporin A, Alfonic TDA-7®, and Olive oil.

Another embodiment comprises cyclosporin A, Alfonic TDA-8®, and Olive oil.

Another embodiment comprises cyclosporin A, Alfonic TDA-9®, and Olive oil.

Another embodiment comprises cyclosporin A, Alkamuls 400-DO®, and Olive oil.

Another embodiment comprises cyclosporin A, Alkamuls 400-MO/E®, and Olive oil.

Another embodiment comprises cyclosporin A, Ameroxol OE-10®, and Olive oil.

Another embodiment comprises cyclosporin A, Ameroxol OE-2®, and Olive oil.

Another embodiment comprises cyclosporin A, Ameroxol OE-20®, and Olive oil.

Another embodiment comprises cyclosporin A, Armix 176®, and Olive oil.

Another embodiment comprises cyclosporin A, Armix 180-C®, and Olive oil.

Another embodiment comprises cyclosporin A, Armix 183®, and Olive oil.

Another embodiment comprises cyclosporin A, Brij 35®, and Olive oil.

Another embodiment comprises cyclosporin A, Brij 35 Liquid®, and Olive oil.

Another embodiment comprises cyclosporin A, Brij 35 SP®, and Olive oil.

Another embodiment comprises cyclosporin A, Canasol MJ 2109®, and Olive oil.

Another embodiment comprises cyclosporin A, Canasol MJ 52®, and Olive oil.

Another embodiment comprises cyclosporin A, Canasol R 3600®, and Olive oil.

Another embodiment comprises cyclosporin A, Canasol R 3603®, and Olive oil.

Another embodiment comprises cyclosporin A, Canasol R 4000 H®, and Olive oil.

Another embodiment comprises cyclosporin A, Cedepal CA-210®, and Olive oil.

Another embodiment comprises cyclosporin A, Cedepal CA-520®, and Olive oil.

Another embodiment comprises cyclosporin A, Cedepal CA-630®, and Olive oil.

Another embodiment comprises cyclosporin A, Cedepal CA-720®, and Olive oil.

Another embodiment comprises cyclosporin A, Cedepal CA-890®, and Olive oil.

Another embodiment comprises cyclosporin A, Cedepal CA-897®, and Olive oil.

Another embodiment comprises cyclosporin A, Cedepal CO-210®, and Olive oil.

Another embodiment comprises cyclosporin A, Cedepal CO-430®, and Olive oil.

Another embodiment comprises cyclosporin A, Cedepal CO-530®, and Olive oil.

Another embodiment comprises cyclosporin A, Cedepal CO-630®, and Olive oil.

Another embodiment comprises cyclosporin A, Cedepal CO-710®, and Olive oil.

Another embodiment comprises cyclosporin A, Cedepal CO-730®, and Olive oil.

Another embodiment comprises cyclosporin A, Cedepal CO-880®, and Olive oil.

Another embodiment comprises cyclosporin A, Cedepal CO-887®, and Olive oil.

Another embodiment comprises cyclosporin A, Cedepal CO-890®, and Olive oil.

Another embodiment comprises cyclosporin A, Cedepal CO-897®, and Olive oil.

Another embodiment comprises cyclosporin A, Cedepal CO-970®, and Olive oil.

Another embodiment comprises cyclosporin A, Cedepal CO-977®, and Olive oil.

Another embodiment comprises cyclosporin A, Cedepal CO-990®, and Olive oil.

Another embodiment comprises cyclosporin A, Cedepal CO-997®, and Olive oil.

Another embodiment comprises cyclosporin A, Chemal BP 261®, and Olive oil.

Another embodiment comprises cyclosporin A, Chemal BP 262®, and Olive oil.

Another embodiment comprises cyclosporin A, Chemal BP 3172®, and Olive oil.

Another embodiment comprises cyclosporin A, Chemal BP 3174®, and Olive oil.

Another embodiment comprises cyclosporin A, Chemal DA-4®, and Olive oil.

Another embodiment comprises cyclosporin A, Chemal DA-6®, and Olive oil.

Another embodiment comprises cyclosporin A, Chemal DA-9®, and Olive oil.

Another embodiment comprises cyclosporin A, Chemal LA-23®, and Olive oil.

Another embodiment comprises cyclosporin A, Chemal LA-4®, and Olive oil.

Another embodiment comprises cyclosporin A, Chemal LA-9®, and Olive oil.

Another embodiment comprises cyclosporin A, Chemal OA-20G®, and Olive oil.

Another embodiment comprises cyclosporin A, Chemal OA-5®, and Olive oil.

Another embodiment comprises cyclosporin A, Chemal OA-9®, and Olive oil.

Another embodiment comprises cyclosporin A, Chemal TDA-12®, and Olive oil.

Another embodiment comprises cyclosporin A, Chemal TDA-15®, and Olive oil.

Another embodiment comprises cyclosporin A, Chemal TDA-3®, and Olive oil.

Another embodiment comprises cyclosporin A, Chemal TDA-6®, and Olive oil.

Another embodiment comprises cyclosporin A, Chemal TDA-9®, and Olive oil.

Another embodiment comprises cyclosporin A, Chemax CO-16®, and Olive oil.

Another embodiment comprises cyclosporin A, Chemax CO-200/50®, and Olive oil.

Another embodiment comprises cyclosporin A, Chemax CO-25®, and Olive oil.

Another embodiment comprises cyclosporin A, Chemax CO-30®, and Olive oil.

Another embodiment comprises cyclosporin A, Chemax CO-36®, and Olive oil.

Another embodiment comprises cyclosporin A, Chemax CO-40®, and Olive oil.

Another embodiment comprises cyclosporin A, Chemax CO-5®, and Olive oil.

Another embodiment comprises cyclosporin A, Chemax CO-80®, and Olive oil.

Another embodiment comprises cyclosporin A, Chemax DNP-150/50®, and Olive oil.

Another embodiment comprises cyclosporin A, Chemax DNP-18®, and Olive oil.

Another embodiment comprises cyclosporin A, Chemax DNP-8®, and Olive oil.

Another embodiment comprises cyclosporin A, Chemax E 200 ML®, and Olive oil.

Another embodiment comprises cyclosporin A, Chemax E 200 MO®, and Olive oil.

Another embodiment comprises cyclosporin A, Chemax E 400 ML®, and Olive oil.

Another embodiment comprises cyclosporin A, Chemax E 400 MO®, and Olive oil.

Another embodiment comprises cyclosporin A, Chemax E 600 ML®, and Olive oil.

Another embodiment comprises cyclosporin A, Chemax E 600 MO®, and Olive oil.

Another embodiment comprises cyclosporin A, Chemax E 1000 MO®, and Olive oil.

Another embodiment comprises cyclosporin A, Chemax E 1000 MS®, and Olive oil.

Another embodiment comprises cyclosporin A, Chemax E200 MS®, and Olive oil.

Another embodiment comprises cyclosporin A, Chemax E400 MS®, and Olive oil.

Another embodiment comprises cyclosporin A, Chemax E600 MS®, and Olive oil.

Another embodiment comprises cyclosporin A, Chemax HCO-16®, and Olive oil.

Another embodiment comprises cyclosporin A, Chemax HCO-200/50®, and Olive oil.

Another embodiment comprises cyclosporin A, Chemax HCO-25®, and Olive oil.

Another embodiment comprises cyclosporin A, Chemax HCO-5®, and Olive oil.

Another embodiment comprises cyclosporin A, Chemax TO-16®, and Olive oil.

Another embodiment comprises cyclosporin A, Chemax TO-8®, and Olive oil.

Another embodiment comprises cyclosporin A, Colalube 3409®, and Olive oil.

Another embodiment comprises cyclosporin A, Colalube 3411®, and Olive oil.

Another embodiment comprises cyclosporin A, Colalube 3413®, and Olive oil.

Another embodiment comprises cyclosporin A, Colalube 3414®, and Olive oil.

Another embodiment comprises cyclosporin A, Colalube 3415®, and Olive oil.

Another embodiment comprises cyclosporin A, Colalube 3416®, and Olive oil.

Another embodiment comprises cyclosporin A, Colalube 3417®, and Olive oil.

Another embodiment comprises cyclosporin A, Delonic NPE-100®, and Olive oil.

Another embodiment comprises cyclosporin A, Delonic NPE-30®, and Olive oil.

Another embodiment comprises cyclosporin A, Delonic NPE-40®, and Olive oil.

Another embodiment comprises cyclosporin A, Delonic OPE-10®, and Olive oil.

Another embodiment comprises cyclosporin A, Delonic OPE-12®, and Olive oil.

Another embodiment comprises cyclosporin A, Delonic OPE-30®, and Olive oil.

Another embodiment comprises cyclosporin A, Delonic OPE-40®, and Olive oil.

Another embodiment comprises cyclosporin A, Delonic OPE-5®, and Olive oil.

Another embodiment comprises cyclosporin A, Delonic OPE-7.5®, and Olive oil.

Another embodiment comprises cyclosporin A, DePeg 16-CO®, and Olive oil.

Another embodiment comprises cyclosporin A, DePeg 25-CO®, and Olive oil.

Another embodiment comprises cyclosporin A, DePeg 30-CO®, and Olive oil.

Another embodiment comprises cyclosporin A, DePeg 40-CO®, and Olive oil.

Another embodiment comprises cyclosporin A, DePeg 5-CO®, and Olive oil.

Another embodiment comprises cyclosporin A, DePeg 80-CO®, and Olive oil.

Another embodiment comprises cyclosporin A, DeSonic S-100®, and Olive oil.

Another embodiment comprises cyclosporin A, DeSonic S-114®, and Olive oil.

Another embodiment comprises cyclosporin A, DeSonic S-405®, and Olive oil.

Another embodiment comprises cyclosporin A, DeSonic S-45®, and Olive oil.

Another embodiment comprises cyclosporin A, DeSonic TDA-9®, and Olive oil.

Another embodiment comprises cyclosporin A, DeThox Acid O-14®, and Olive oil.

Another embodiment comprises cyclosporin A, DeThox Acid O-16®, and Olive oil.

Another embodiment comprises cyclosporin A, DeThox Acid O-9®, and Olive oil.

Another embodiment comprises cyclosporin A, DeThox Acid TO-14®, and Olive oil.

Another embodiment comprises cyclosporin A, DeThox Acid TO-16.5®, and Olive oil.

Another embodiment comprises cyclosporin A, DeThox Acid TO-8.5®, and Olive oil.

Another embodiment comprises cyclosporin A, Dur-Em 114®, and Olive oil.

Another embodiment comprises cyclosporin A, Dur-Em 117®, and Olive oil.

Another embodiment comprises cyclosporin A, Dur-Em 204®, and Olive oil.

Another embodiment comprises cyclosporin A, Dur-Em 207®, and Olive oil.

Another embodiment comprises cyclosporin A, Dur-Em 207E®, and Olive oil.

Another embodiment comprises cyclosporin A, Emerest 2704®, and Olive oil.

Another embodiment comprises cyclosporin A, Emerest 2712®, and Olive oil.

Another embodiment comprises cyclosporin A, Emerest 2715®, and Olive oil.

Another embodiment comprises cyclosporin A, Ethal EH-2®, and Olive oil.

Another embodiment comprises cyclosporin A, Ethal EH-5®, and Olive oil.

Another embodiment comprises cyclosporin A, Ethal OA-23®, and Olive oil.

Another embodiment comprises cyclosporin A, Ethosperse CA-20®, and Olive oil.

Another embodiment comprises cyclosporin A, Ethosperse LA-12®, and Olive oil.

Another embodiment comprises cyclosporin A, Ethosperse LA-23®, and Olive oil.

Another embodiment comprises cyclosporin A, Ethosperse LA-4®, and Olive oil.

Another embodiment comprises cyclosporin A, Ethox 3095®, and Olive oil.

Another embodiment comprises cyclosporin A, Ethox CO-16®, and Olive oil.

Another embodiment comprises cyclosporin A, Ethox CO-200®, and Olive oil.

Another embodiment comprises cyclosporin A, Ethox CO-25®, and Olive oil.

Another embodiment comprises cyclosporin A, Ethox CO-30®, and Olive oil.

Another embodiment comprises cyclosporin A, Ethox CO-36®, and Olive oil.

Another embodiment comprises cyclosporin A, Ethox CO-40®, and Olive oil.

Another embodiment comprises cyclosporin A, Ethox CO-5®, and Olive oil.

Another embodiment comprises cyclosporin A, Ethox DO-14®, and Olive oil.

Another embodiment comprises cyclosporin A, Ethox DO-9®, and Olive oil.

Another embodiment comprises cyclosporin A, Ethox MA-8®, and Olive oil.

Another embodiment comprises cyclosporin A, Ethox Ml-14®, and Olive oil.

Another embodiment comprises cyclosporin A, Ethox ML-14®, and Olive oil.

Another embodiment comprises cyclosporin A, Ethox ML-5®, and Olive oil.

Another embodiment comprises cyclosporin A, Ethox MO-14®, and Olive oil.

Another embodiment comprises cyclosporin A, Ethox MO-9®, and Olive oil.

Another embodiment comprises cyclosporin A, Ethox MS-14®, and Olive oil.

Another embodiment comprises cyclosporin A, Ethox MS-23®, and Olive oil.

Another embodiment comprises cyclosporin A, Ethox MS-40®, and Olive oil.

Another embodiment comprises cyclosporin A, Ethox MS-8®, and Olive oil.

Another embodiment comprises cyclosporin A, Ethox TO-16®, and Olive oil.

Another embodiment comprises cyclosporin A, Ethox TO-8®, and Olive oil.

Another embodiment comprises cyclosporin A, Ethylan SN-120®, and Olive oil.

Another embodiment comprises cyclosporin A, Ethylan SN-70®, and Olive oil.

Another embodiment comprises cyclosporin A, Ethylan SN-90®, and Olive oil.

Another embodiment comprises cyclosporin A, Flexricin 13®, and Olive oil.

Another embodiment comprises cyclosporin A, Flexricin 15®, and Olive oil.

Another embodiment comprises cyclosporin A, Genapol 1261®, and Olive oil.

Another embodiment comprises cyclosporin A, Generol 122 E25®, and Olive oil.

Another embodiment comprises cyclosporin A, Glicepol 160®, and Olive oil.

Another embodiment comprises cyclosporin A, Glicepol 560®, and Olive oil.

Another embodiment comprises cyclosporin A, Glicepol GMS 20®, and Olive oil.

Another embodiment comprises cyclosporin A, Hetoxide BN-13®, and Olive oil.

Another embodiment comprises cyclosporin A, Hetoxide DNP-4®, and Olive oil.

Another embodiment comprises cyclosporin A, Hetoxide DNP-9.6®, and Olive oil.

Another embodiment comprises cyclosporin A, Hetoxide G-26®, and Olive oil.

Another embodiment comprises cyclosporin A, Hetoxide G-7®, and Olive oil.

Another embodiment comprises cyclosporin A, Hetoxide GT-20®, and Olive oil.

Another embodiment comprises cyclosporin A, Hetoxide HC-40®, and Olive oil.

Another embodiment comprises cyclosporin A, Hetoxol C-24®, and Olive oil.

Another embodiment comprises cyclosporin A, Hetoxol CA-10®, and Olive oil.

Another embodiment comprises cyclosporin A, Hetoxol CA-2®, and Olive oil.

Another embodiment comprises cyclosporin A, Hetoxol CA-20®, and Olive oil.

Another embodiment comprises cyclosporin A, Hetoxol CAWS®, and Olive oil.

Another embodiment comprises cyclosporin A, Hetoxol CD-3®, and Olive oil.

Another embodiment comprises cyclosporin A, Hetoxol CD-4®, and Olive oil.

Another embodiment comprises cyclosporin A, Hetoxol CS-15®, and Olive oil.

Another embodiment comprises cyclosporin A, Hetoxol CS-20D®, and Olive oil.

Another embodiment comprises cyclosporin A, Hetoxol CS-20D®, and Olive oil.

Another embodiment comprises cyclosporin A, Hetoxol CS-25®, and Olive oil.

Another embodiment comprises cyclosporin A, Hetoxol CS-50®, and Olive oil.

Another embodiment comprises cyclosporin A, Hetoxol CS-9®, and Olive oil.

Another embodiment comprises cyclosporin A, Hetoxol CSA-15®, and Olive oil.

Another embodiment comprises cyclosporin A, Hetoxol D®, and Olive oil.

Another embodiment comprises cyclosporin A, Hetoxol G®, and Olive oil.

Another embodiment comprises cyclosporin A, Hetoxol J®, and Olive oil.

Another embodiment comprises cyclosporin A, Hetoxol L®, and Olive oil.

Another embodiment comprises cyclosporin A, Hetoxol L-23®, and Olive oil.

Another embodiment comprises cyclosporin A, Hetoxol L-4®, and Olive oil.

Another embodiment comprises cyclosporin A, Hetoxol L-9®, and Olive oil.

Another embodiment comprises cyclosporin A, Hetoxol LS-9®, and Olive oil.

Another embodiment comprises cyclosporin A, Hetoxol M-3®, and Olive oil.

Another embodiment comprises cyclosporin A, Hetoxol OA-10 Special®, and Olive oil.

Another embodiment comprises cyclosporin A, Hetoxol OA-20 Special®, and Olive oil.

Another embodiment comprises cyclosporin A, Hetoxol OA-3 Special®, and Olive oil.

Another embodiment comprises cyclosporin A, Hetoxol OA-5 Special®, and Olive oil.

Another embodiment comprises cyclosporin A, Hetoxol OL-10®, and Olive oil.

Another embodiment comprises cyclosporin A, Hetoxol OL-2®, and Olive oil.

Another embodiment comprises cyclosporin A, Hetoxol OL-23®, and Olive oil.

Another embodiment comprises cyclosporin A, Hetoxol OL-4®, and Olive oil.

Another embodiment comprises cyclosporin A, Hetoxol OL-40®, and Olive oil.

Another embodiment comprises cyclosporin A, Hetoxol OL-5®, and Olive oil.

Another embodiment comprises cyclosporin A, Hetoxol PLA®, and Olive oil.

Another embodiment comprises cyclosporin A, Hetoxol SP-15®, and Olive oil.

Another embodiment comprises cyclosporin A, Hetoxol STA-10®, and Olive oil.

Another embodiment comprises cyclosporin A, Hetoxol STA-2®, and Olive oil.

Another embodiment comprises cyclosporin A, Hetoxol STA-30®, and Olive oil.

Another embodiment comprises cyclosporin A, Hetoxol TD-12®, and Olive oil.

Another embodiment comprises cyclosporin A, Hetoxol TD-18®, and Olive oil.

Another embodiment comprises cyclosporin A, Hetoxol TD-3®, and Olive oil.

Another embodiment comprises cyclosporin A, Hetoxol TD-6®, and Olive oil.

Another embodiment comprises cyclosporin A, Hetoxol TD-9®, and Olive oil.

Another embodiment comprises cyclosporin A, Hetoxol TDEP-15®, and Olive oil.

Another embodiment comprises cyclosporin A, Hetoxol TDEP-63®, and Olive oil.

Another embodiment comprises cyclosporin A, Iconol 24-12®, and Olive oil.

Another embodiment comprises cyclosporin A, Iconol 24-23®, and Olive oil.

Another embodiment comprises cyclosporin A, Iconol 24-4®, and Olive oil.

Another embodiment comprises cyclosporin A, Iconol DA-4®, and Olive oil.

Another embodiment comprises cyclosporin A, Iconol DA-6®, and Olive oil.

Another embodiment comprises cyclosporin A, Iconol TDA-10®, and Olive oil.

Another embodiment comprises cyclosporin A, Iconol TDA-3®, and Olive oil.

Another embodiment comprises cyclosporin A, Iconol TDA-6®, and Olive oil.

Another embodiment comprises cyclosporin A, Iconol TDA-8-90%®, and Olive oil.

Another embodiment comprises cyclosporin A, Iconol TDA-9®, and Olive oil.

Another embodiment comprises cyclosporin A, Igepal CA-210®, and Olive oil.

Another embodiment comprises cyclosporin A, Igepal CA-407®, and Olive oil.

Another embodiment comprises cyclosporin A, Igepal CA-420®, and Olive oil.

Another embodiment comprises cyclosporin A, Igepal CA-520®, and Olive oil.

Another embodiment comprises cyclosporin A, Igepal CA-620®, and Olive oil.

Another embodiment comprises cyclosporin A, Igepal CA-630®, and Olive oil.

Another embodiment comprises cyclosporin A, Igepal CA-720®, and Olive oil.

Another embodiment comprises cyclosporin A, Igepal CA-877®, and Olive oil.

Another embodiment comprises cyclosporin A, Igepal CA-887®, and Olive oil.

Another embodiment comprises cyclosporin A, Igepal CA-897®, and Olive oil.

Another embodiment comprises cyclosporin A, Igepal CO210®, and Olive oil.

Another embodiment comprises cyclosporin A, Igepal CO-430®, and Olive oil.

Another embodiment comprises cyclosporin A, Igepal CO-520®, and Olive oil.

Another embodiment comprises cyclosporin A, Igepal CO-530®, and Olive oil.

Another embodiment comprises cyclosporin A, Igepal CO-610®, and Olive oil.

Another embodiment comprises cyclosporin A, Igepal CO-630®, and Olive oil.

Another embodiment comprises cyclosporin A, Igepal CO-630 Special®, and Olive oil.

Another embodiment comprises cyclosporin A, Igepal CO-660®, and Olive oil.

Another embodiment comprises cyclosporin A, Igepal CO-710®, and Olive oil.

Another embodiment comprises cyclosporin A, Igepal CO-720®, and Olive oil.

Another embodiment comprises cyclosporin A, Igepal CO-730®, and Olive oil.

Another embodiment comprises cyclosporin A, Igepal CO-738®, and Olive oil.

Another embodiment comprises cyclosporin A, Igepal CO-850®, and Olive oil.

Another embodiment comprises cyclosporin A, Igepal CO-880®, and Olive oil.

Another embodiment comprises cyclosporin A, Igepal CO-887®, and Olive oil.

Another embodiment comprises cyclosporin A, Igepal CO-890®, and Olive oil.

Another embodiment comprises cyclosporin A, Igepal CO-897®, and Olive oil.

Another embodiment comprises cyclosporin A, Igepal CO-970®, and Olive oil.

Another embodiment comprises cyclosporin A, Igepal CO-977®, and Olive oil.

Another embodiment comprises cyclosporin A, Igepal CO-987®, and Olive oil.

Another embodiment comprises cyclosporin A, Igepal CO-990 FLK®, and Olive oil.

Another embodiment comprises cyclosporin A, Igepal CO-997®, and Olive oil.

Another embodiment comprises cyclosporin A, Igepal DM-430®, and Olive oil.

Another embodiment comprises cyclosporin A, Igepal DM-530®, and Olive oil.

Another embodiment comprises cyclosporin A, Igepal DM-710®, and Olive oil.

Another embodiment comprises cyclosporin A, Igepal DM-730®, and Olive oil.

Another embodiment comprises cyclosporin A, Igepal DM-880®, and Olive oil.

Another embodiment comprises cyclosporin A, Igepal DM-970 FLK®, and Olive oil.

Another embodiment comprises cyclosporin A, Igepal OD-410®, and Olive oil.

Another embodiment comprises cyclosporin A, Igepal RC-520®, and Olive oil.

Another embodiment comprises cyclosporin A, Igepal RC-620®, and Olive oil.

Another embodiment comprises cyclosporin A, Igepal RC-630®, and Olive oil.

Another embodiment comprises cyclosporin A, Lexemul CS-20®, and Olive oil.

Another embodiment comprises cyclosporin A, Lipo GMS 450®, and Olive oil.

Another embodiment comprises cyclosporin A, Lipo Polyglycol 1000®, and Olive oil.

Another embodiment comprises cyclosporin A, Lipo Polyglycol 200®, and Olive oil.

Another embodiment comprises cyclosporin A, Lipo Polyglycol 300®, and Olive oil.

Another embodiment comprises cyclosporin A, Lipo Polyglycol 3350®, and Olive oil.

Another embodiment comprises cyclosporin A, Lipo Polyglycol 400®, and Olive oil.

Another embodiment comprises cyclosporin A, Lipo Polyglycol 600®, and Olive oil.

Another embodiment comprises cyclosporin A, Lipocol C-10®, and Olive oil.

Another embodiment comprises cyclosporin A, Lipocol C-2®, and Olive oil.

Another embodiment comprises cyclosporin A, Lipocol C-20®, and Olive oil.

Another embodiment comprises cyclosporin A, Lipocol L-12®, and Olive oil.

Another embodiment comprises cyclosporin A, Lipocol L-23®, and Olive oil.

Another embodiment comprises cyclosporin A, Lipocol L-4®, and Olive oil.

Another embodiment comprises cyclosporin A, Lipocol O-10®, and Olive oil.

Another embodiment comprises cyclosporin A, Lipocol O-2®, and Olive oil.

Another embodiment comprises cyclosporin A, Lipocol O-20®, and Olive oil.

Another embodiment comprises cyclosporin A, Lipocol O-5®, and Olive oil.

Another embodiment comprises cyclosporin A, Lipocol S-10®, and Olive oil.

Another embodiment comprises cyclosporin A, Lipocol S-2®, and Olive oil.

Another embodiment comprises cyclosporin A, Lipocol S-20®, and Olive oil.

Another embodiment comprises cyclosporin A, Lipocol SC-15®, and Olive oil.

Another embodiment comprises cyclosporin A, Lipocol SC-20®, and Olive oil.

Another embodiment comprises cyclosporin A, Lipocol SC-4®, and Olive oil.

Another embodiment comprises cyclosporin A, Lipocol TD-12®, and Olive oil.

Another embodiment comprises cyclosporin A, Lumulse 40-O K®, and Olive oil.

Another embodiment comprises cyclosporin A, Lumulse 42-OK®, and Olive oil.

Another embodiment comprises cyclosporin A, Lumulse GMT-40®, and Olive oil.

Another embodiment comprises cyclosporin A, Lumulse PEG 1450®, and Olive oil.

Another embodiment comprises cyclosporin A, Lumulse PEG 1450 NF®, and Olive oil.

Another embodiment comprises cyclosporin A, Lumulse PEG 8000®, and Olive oil.

Another embodiment comprises cyclosporin A, Lumulse PGO®, and Olive oil.

Another embodiment comprises cyclosporin A, Lumulse POE (2) Oleyl Amine®, and Olive oil.

Another embodiment comprises cyclosporin A, Magrabar PGO-104®, and Olive oil.

Another embodiment comprises cyclosporin A, Mapeg 200 ML®, and Olive oil.

Another embodiment comprises cyclosporin A, Mapeg 400 MO®, and Olive oil.

Another embodiment comprises cyclosporin A, Mapeg 400 MOT®, and Olive oil.

Another embodiment comprises cyclosporin A, Mapeg 400DO®, and Olive oil.

Another embodiment comprises cyclosporin A, Mapeg 400DOT®, and Olive oil.

Another embodiment comprises cyclosporin A, Mapeg 600DOT®, and Olive oil.

Another embodiment comprises cyclosporin A, Marlox B 24/80®, and Olive oil.

Another embodiment comprises cyclosporin A, Marlox FK 86®, and Olive oil.

Another embodiment comprises cyclosporin A, Marlox MO 154®, and Olive oil.

Another embodiment comprises cyclosporin A, Mazox CDA CG®, and Olive oil.

Another embodiment comprises cyclosporin A, Monoglycerides Glyceryl Monestearate®, and Olive oil.

Another embodiment comprises cyclosporin A, Myrj 45®, and Olive oil.

Another embodiment comprises cyclosporin A, Myrj 52®, and Olive oil.

Another embodiment comprises cyclosporin A, Myrj 52S®, and Olive oil.

Another embodiment comprises cyclosporin A, Myrj 53®, and Olive oil.

Another embodiment comprises cyclosporin A, Myrj 59®, and Olive oil.

Another embodiment comprises cyclosporin A, Norfox NP-12®, and Olive oil.

Another embodiment comprises cyclosporin A, Norox NP-4®, and Olive oil.

Another embodiment comprises cyclosporin A, Norfox NP-6®, and Olive oil.

Another embodiment comprises cyclosporin A, Norfox NP-887®, and Olive oil.

Another embodiment comprises cyclosporin A, Norfox NP-9®, and Olive oil.

Another embodiment comprises cyclosporin A, Norfox NP-977®, and Olive oil.

Another embodiment comprises cyclosporin A, Norfox OP-100®, and Olive oil.

Another embodiment comprises cyclosporin A, Norfox OP-102®, and Olive oil.

Another embodiment comprises cyclosporin A, Norfox OP-114®, and Olive oil.

Another embodiment comprises cyclosporin A, Norfox OP-45®, and Olive oil.

Another embodiment comprises cyclosporin A, Pegosperse 100-L®, and Olive oil.

Another embodiment comprises cyclosporin A, Pegosperse 100-O®, and Olive oil.

Another embodiment comprises cyclosporin A, Pegosperse 100-S®, and Olive oil.

Another embodiment comprises cyclosporin A, Pegosperse 1500-MS®, and Olive oil.

Another embodiment comprises cyclosporin A, Pegosperse 1750-MS®, and Olive oil.

Another embodiment comprises cyclosporin A, Pegosperse 200 DL®, and Olive oil.

Another embodiment comprises cyclosporin A, Pegosperse 200-ML®, and Olive oil.

Another embodiment comprises cyclosporin A, Pegosperse 400-DL®, and Olive oil.

Another embodiment comprises cyclosporin A, Pegosperse 400-DO®, and Olive oil.

Another embodiment comprises cyclosporin A, Pegosperse 400-DS®, and Olive oil.

Another embodiment comprises cyclosporin A, Pegosperse 400-ML®, and Olive oil.

Another embodiment comprises cyclosporin A, Pegosperse 400-MO®, and Olive oil.

Another embodiment comprises cyclosporin A, Pegosperse 400-MOT®, and Olive oil.

Another embodiment comprises cyclosporin A, Pegosperse 400-MS®, and Olive oil.

Another embodiment comprises cyclosporin A, Pegosperse 50-DS®, and Olive oil.

Another embodiment comprises cyclosporin A, Pegosperse 50-MS®, and Olive oil.

Another embodiment comprises cyclosporin A, Pegosperse 600-ML®, and Olive oil.

Another embodiment comprises cyclosporin A, Pegosperse 600-MS®, and Olive oil.

Another embodiment comprises cyclosporin A, Plurafac A-38®, and Olive oil.

Another embodiment comprises cyclosporin A, Plurafac B-25-5®, and Olive oil.

Another embodiment comprises cyclosporin A, Plurafac B-26®, and Olive oil.

Another embodiment comprises cyclosporin A, Plurafac D-25®, and Olive oil.

Another embodiment comprises cyclosporin A, Plurafac LF 131®, and Olive oil.

Another embodiment comprises cyclosporin A, Plurafac LF 4030®, and Olive oil.

Another embodiment comprises cyclosporin A, Plurafac LF 7000®, and Olive oil.

Another embodiment comprises cyclosporin A, Plurafac RA-20®, and Olive oil.

Another embodiment comprises cyclosporin A, Plurafac RA-30®, and Olive oil.

Another embodiment comprises cyclosporin A, Plurafac RA-40®, and Olive oil.

Another embodiment comprises cyclosporin A, Pluronic 10R5®, and Olive oil.

Another embodiment comprises cyclosporin A, Pluronic 17R2®, and Olive oil.

Another embodiment comprises cyclosporin A, Pluronic 17R4®, and Olive oil.

Another embodiment comprises cyclosporin A, Pluronic 25R2®, and Olive oil.

Another embodiment comprises cyclosporin A, Pluronic 25R4®, and Olive oil.

Another embodiment comprises cyclosporin A, Pluronic 31R1®, and Olive oil.

Another embodiment comprises cyclosporin A, Pluronic F108®, and Olive oil.

Another embodiment comprises cyclosporin A, Pluronic F127®, and Olive oil.

Another embodiment comprises cyclosporin A, Pluronic F38®, and Olive oil.

Another embodiment comprises cyclosporin A, Pluronic F68®, and Olive oil.

Another embodiment comprises cyclosporin A, Pluronic F68LF®, and Olive oil.

Another embodiment comprises cyclosporin A, Pluronic F77®, and Olive oil.

Another embodiment comprises cyclosporin A, Pluronic F87®, and Olive oil.

Another embodiment comprises cyclosporin A, Pluronic F88®, and Olive oil.

Another embodiment comprises cyclosporin A, Pluronic F98®, and Olive oil.

Another embodiment comprises cyclosporin A, Pluronic L10®, and Olive oil.

Another embodiment comprises cyclosporin A, Pluronic L101®, and Olive oil.

Another embodiment comprises cyclosporin A, Pluronic L121®, and Olive oil.

Another embodiment comprises cyclosporin A, Pluronic L31®, and Olive oil.

Another embodiment comprises cyclosporin A, Pluronic L35®, and Olive oil.

Another embodiment comprises cyclosporin A, Pluronic L43®, and Olive oil.

Another embodiment comprises cyclosporin A, Pluronic L44®, and Olive oil.

Another embodiment comprises cyclosporin A, Pluronic L61®, and Olive oil.

Another embodiment comprises cyclosporin A, Pluronic L62®, and Olive oil.

Another embodiment comprises cyclosporin A, Pluronic L62D®, and Olive oil.

Another embodiment comprises cyclosporin A, Pluronic L62LF®, and Olive oil.

Another embodiment comprises cyclosporin A, Pluronic L64®, and Olive oil.

Another embodiment comprises cyclosporin A, Pluronic L81®, and Olive oil.

Another embodiment comprises cyclosporin A, Pluronic L92®, and Olive oil.

Another embodiment comprises cyclosporin A, Pluronic P103®, and Olive oil.

Another embodiment comprises cyclosporin A, Pluronic P104®, and Olive oil.

Another embodiment comprises cyclosporin A, Pluronic P105®, and Olive oil.

Another embodiment comprises cyclosporin A, Pluronic P123®, and Olive oil.

Another embodiment comprises cyclosporin A, Pluronic P65®, and Olive oil.

Another embodiment comprises cyclosporin A, Pluronic P84®, and Olive oil.

Another embodiment comprises cyclosporin A, Pluronic P85®, and Olive oil.

Another embodiment comprises cyclosporin A, Polyaldo 10-2-P®, and Olive oil.

Another embodiment comprises cyclosporin A, Polyaldo DGDO®, and Olive oil.

Another embodiment comprises cyclosporin A, Polyaldo HGDS®, and Olive oil.

Another embodiment comprises cyclosporin A, Polyaldo TGMS®, and Olive oil.

Another embodiment comprises cyclosporin A, Protegin V®, and Olive oil.

Another embodiment comprises cyclosporin A, Protegin W®, and Olive oil.

Another embodiment comprises cyclosporin A, Protegin WX®, and Olive oil.

Another embodiment comprises cyclosporin A, Protegin XV®, and Olive oil.

Another embodiment comprises cyclosporin A, Rhodasurf BC-420®, and Olive oil.

Another embodiment comprises cyclosporin A, Rhodasurf BC-610®, and Olive oil.

Another embodiment comprises cyclosporin A, Rhodasurf BC-630®, and Olive oil.

Another embodiment comprises cyclosporin A, Rhodasurf BC-720®, and Olive oil.

Another embodiment comprises cyclosporin A, Rhodasurf BC-840®, and Olive oil.

Another embodiment comprises cyclosporin A, Rhodasurf L 1®, and Olive oil.

Another embodiment comprises cyclosporin A, Rhodasurf L 12®, and Olive oil.

Another embodiment comprises cyclosporin A, Rhodasurf L 2®, and Olive oil.

Another embodiment comprises cyclosporin A, Rhodasurf L 20®, and Olive oil.

Another embodiment comprises cyclosporin A, Rhodasurf L 3®, and Olive oil.

Another embodiment comprises cyclosporin A, Rhodasurf L 30®, and Olive oil.

Another embodiment comprises cyclosporin A, Rhodasurf L 4®, and Olive oil.

Another embodiment comprises cyclosporin A, Rhodasurf L 50®, and Olive oil.

Another embodiment comprises cyclosporin A, Rhodasurf L 7/90®, and Olive oil.

Another embodiment comprises cyclosporin A, Rhodasurf LA-3®, and Olive oil.

Another embodiment comprises cyclosporin A, Rhodasurf LA-7®, and Olive oil.

Another embodiment comprises cyclosporin A, Rhodasurf LAN-23®, and Olive oil.

Another embodiment comprises cyclosporin A, Rhodasurf ON-870®, and Olive oil.

Another embodiment comprises cyclosporin A, Rhodasurf ON-877®, and Olive oil.

Another embodiment comprises cyclosporin A, Rhodasurf TDA-8/5®, and Olive oil.

Another embodiment comprises cyclosporin A, Ritapro 100®, and Olive oil.

Another embodiment comprises cyclosporin A, Ritapro 165®, and Olive oil.

Another embodiment comprises cyclosporin A, Ritapro 200®, and Olive oil.

Another embodiment comprises cyclosporin A, Ritapro 300®, and Olive oil.

Another embodiment comprises cyclosporin A, Ritoleth 10®, and Olive oil.

Another embodiment comprises cyclosporin A, Ritoleth 2®, and Olive oil.

Another embodiment comprises cyclosporin A, Ritoleth 20®, and Olive oil.

Another embodiment comprises cyclosporin A, Ritoleth 5®, and Olive oil.

Another embodiment comprises cyclosporin A, Santone 10-10-0®, and Olive oil.

Another embodiment comprises cyclosporin A, Santone 3-1-S XTR®, and Olive oil.

Another embodiment comprises cyclosporin A, Santone 3-1 -SH®, and Olive oil.

Another embodiment comprises cyclosporin A, Santone 8-1-O®, and Olive oil.

Another embodiment comprises cyclosporin A, Solulan 16®, and Olive oil.

Another embodiment comprises cyclosporin A, Solulan 75®, and Olive oil.

Another embodiment comprises cyclosporin A, Solulan C-24®, and Olive oil.

Another embodiment comprises cyclosporin A, Solulan L-575®, and Olive oil.

Another embodiment comprises cyclosporin A, Standamox CAW®, and Olive oil.

Another embodiment comprises cyclosporin A, Surfine AZI-A®, and Olive oil.

Another embodiment comprises cyclosporin A, Surfonic CO-15®, and Olive oil.

Another embodiment comprises cyclosporin A, Surfonic CO-25®, and Olive oil.

Another embodiment comprises cyclosporin A, Surfonic CO-30®, and Olive oil.

Another embodiment comprises cyclosporin A, Surfonic CO-36®, and Olive oil.

Another embodiment comprises cyclosporin A, Surfonic CO-42®, and Olive oil.

Another embodiment comprises cyclosporin A, Surfonic E400-MO®, and Olive oil.

Another embodiment comprises cyclosporin A, Surfonic L 12-3®, and Olive oil.

Another embodiment comprises cyclosporin A, Surfonic L 12-6®, and Olive oil.

Another embodiment comprises cyclosporin A, Surfonic L 12-8®, and Olive oil.

Another embodiment comprises cyclosporin A, Surfonic L24-12®, and Olive oil.

Another embodiment comprises cyclosporin A, Surfonic L24-2®, and Olive oil.

Another embodiment comprises cyclosporin A, Surfonic L24-22®, and Olive oil.

Another embodiment comprises cyclosporin A, Surfonic L24-3®, and Olive oil.

Another embodiment comprises cyclosporin A, Surfonic L24-4®, and Olive oil.

Another embodiment comprises cyclosporin A, Surfonic L24-5®, and Olive oil.

Another embodiment comprises cyclosporin A, Surfonic L24-7®, and Olive oil.

Another embodiment comprises cyclosporin A, Surfonic L24-7.1®, and Olive oil.

Another embodiment comprises cyclosporin A, Surfonic L24-9®, and Olive oil.

Another embodiment comprises cyclosporin A, Surfonic LF-17®, and Olive oil.

Another embodiment comprises cyclosporin A, Surfonic LF-18®, and Olive oil.

Another embodiment comprises cyclosporin A, Surfonic LF-37®, and Olive oil.

Another embodiment comprises cyclosporin A, Surfonic LF-41®, and Olive oil.

Another embodiment comprises cyclosporin A, Surfonic LF-42®, and Olive oil.

Another embodiment comprises cyclosporin A, Surfonic LF-47®, and Olive oil.

Another embodiment comprises cyclosporin A, Surfonic LF-68®, and Olive oil.

Another embodiment comprises cyclosporin A, Surfonic N-10®, and Olive oil.

Another embodiment comprises cyclosporin A, Surfonic N-100®, and Olive oil.

Another embodiment comprises cyclosporin A, Surfonic N-102®, and Olive oil.

Another embodiment comprises cyclosporin A, Surfonic N-120®, and Olive oil.

Another embodiment comprises cyclosporin A, Surfonic N-150®, and Olive oil.

Another embodiment comprises cyclosporin A, Surfonic N-200®, and Olive oil.

Another embodiment comprises cyclosporin A, Surfonic N-300®, and Olive oil.

Another embodiment comprises cyclosporin A, Surfonic N-31.5®, and Olive oil.

Another embodiment comprises cyclosporin A, Surfonic N-40®, and Olive oil.

Another embodiment comprises cyclosporin A, Surfonic N-400®, and Olive oil.

Another embodiment comprises cyclosporin A, Surfonic N-60®, and Olive oil.

Another embodiment comprises cyclosporin A, Surfonic N-85®, and Olive oil.

Another embodiment comprises cyclosporin A, Surfonic N-95®, and Olive oil.

Another embodiment comprises cyclosporin A, Surfonic NB-18®, and Olive oil.

Another embodiment comprises cyclosporin A, Surfonic NB-23®, and Olive oil.

Another embodiment comprises cyclosporin A, Surfonic NB-25®, and Olive oil.

Another embodiment comprises cyclosporin A, Surfonic NB-307®, and Olive oil.

Another embodiment comprises cyclosporin A, Surfonic NB-31®, and Olive oil.

Another embodiment comprises cyclosporin A, Surfonic NB-41®, and Olive oil.

Another embodiment comprises cyclosporin A, Surfonic NB-43®, and Olive oil.

Another embodiment comprises cyclosporin A, Surfonic OP-100®, and Olive oil.

Another embodiment comprises cyclosporin A, Surfonic OP-120®, and Olive oil.

Another embodiment comprises cyclosporin A, Surfonic OP-15®, and Olive oil.

Another embodiment comprises cyclosporin A, Surfonic OP-35®, and Olive oil.

Another embodiment comprises cyclosporin A, Surfonic OP-70®, and Olive oil.

Another embodiment comprises cyclosporin A, Surfonic P1®, and Olive oil.

Another embodiment comprises cyclosporin A, Surfonic P3®, and Olive oil.

Another embodiment comprises cyclosporin A, Surfonic P5®, and Olive oil.

Another embodiment comprises cyclosporin A, Surfonic P6®, and Olive oil.

Another embodiment comprises cyclosporin A, Surfonic POA-17R2®, and Olive oil.

Another embodiment comprises cyclosporin A, Surfonic POA-25R2®, and Olive oil.

Another embodiment comprises cyclosporin A, Surfonic TDA-11®, and Olive oil.

Another embodiment comprises cyclosporin A, Surfonic TDA-3B®, and Olive oil.

Another embodiment comprises cyclosporin A, Surfonic TDA-6®, and Olive oil.

Another embodiment comprises cyclosporin A, Surfonic TDA-6/88®, and Olive oil.

Another embodiment comprises cyclosporin A, Surfonic TDA-8®, and Olive oil.

Another embodiment comprises cyclosporin A, Surfonic TDA-8/90®, and Olive oil.

Another embodiment comprises cyclosporin A, Surfonic TDA-9®, and Olive oil.

Another embodiment comprises cyclosporin A, Surfonic TX-CCN®, and Olive oil.

Another embodiment comprises cyclosporin A, Tagat CH 40®, and Olive oil.

Another embodiment comprises cyclosporin A, Tagat CH 60®, and Olive oil.

Another embodiment comprises cyclosporin A, Tagat L 2®, and Olive oil.

Another embodiment comprises cyclosporin A, Tagat S®, and Olive oil.

Another embodiment comprises cyclosporin A, Tagat S 2®, and Olive oil.

Another embodiment comprises cyclosporin A, Tally 100 Plus®, and Olive oil.

Another embodiment comprises cyclosporin A, T-Det A106®, and Olive oil.

Another embodiment comprises cyclosporin A, T-Det A109®, and Olive oil.

Another embodiment comprises cyclosporin A, T-Det A136®, and Olive oil.

Another embodiment comprises cyclosporin A, T-Det A139®, and Olive oil.

Another embodiment comprises cyclosporin A, T-Det A2412®, and Olive oil.

Another embodiment comprises cyclosporin A, T-Det A243®, and Olive oil.

Another embodiment comprises cyclosporin A, T-Det A247®, and Olive oil.

Another embodiment comprises cyclosporin A, T-Det A249®, and Olive oil.

Another embodiment comprises cyclosporin A, T-Det A467®, and Olive oil.

Another embodiment comprises cyclosporin A, T-Det COE®, and Olive oil.

Another embodiment comprises cyclosporin A, T-Det DD-10®, and Olive oil.

Another embodiment comprises cyclosporin A, T-Det DD-5®, and Olive oil.

Another embodiment comprises cyclosporin A, T-Det DD-7®, and Olive oil.

Another embodiment comprises cyclosporin A, T-Det EPO-104®, and Olive oil.

Another embodiment comprises cyclosporin A, T-Det EPO-61®, and Olive oil.

Another embodiment comprises cyclosporin A, T-Det EPO-62®, and Olive oil.

Another embodiment comprises cyclosporin A, T-Det EPO-64®, and Olive oil.

Another embodiment comprises cyclosporin A, T-Det N-1.5®, and Olive oil.

Another embodiment comprises cyclosporin A, T-Det N-10.5®, and Olive oil.

Another embodiment comprises cyclosporin A, T-Det N-100®, and Olive oil.

Another embodiment comprises cyclosporin A, T-Det N-1007®, and Olive oil.

Another embodiment comprises cyclosporin A, T-Det N-12®, and Olive oil.

Another embodiment comprises cyclosporin A, T-Det N-14®, and Olive oil.

Another embodiment comprises cyclosporin A, T-Det N-20®, and Olive oil.

Another embodiment comprises cyclosporin A, T-Det N-30®, and Olive oil.

Another embodiment comprises cyclosporin A, T-Det N-307®, and. Olive oil.

Another embodiment comprises cyclosporin A, T-Det N-4®, and Olive oil.

Another embodiment comprises cyclosporin A, T-Det N-40®, and Olive oil.

Another embodiment comprises cyclosporin A, T-Det N-407®, and Olive oil.

Another embodiment comprises cyclosporin A, T-Det N-50®, and Olive oil.

Another embodiment comprises cyclosporin A, T-Det N-507®, and Olive oil.

Another embodiment comprises cyclosporin A, T-Det N-6®, and Olive oil.

Another embodiment comprises cyclosporin A, T-Det N-70®, and Olive oil.

Another embodiment comprises cyclosporin A, T-Det N-8®, and Olive oil.

Another embodiment comprises cyclosporin A, T-Det N-9.5®, and Olive oil.

Another embodiment comprises cyclosporin A, T-Det O-12®, and Olive oil.

Another embodiment comprises cyclosporin A, T-Det O-165®, and Olive oil.

Another embodiment comprises cyclosporin A, T-Det O-307®, and Olive oil.

Another embodiment comprises cyclosporin A, T-Det O-40®, and Olive oil.

Another embodiment comprises cyclosporin A, T-Det O-407®, and Olive oil.

Another embodiment comprises cyclosporin A, T-Det O-6®, and Olive oil.

Another embodiment comprises cyclosporin A, T-Det O-8®, and Olive oil.

Another embodiment comprises cyclosporin A, T-Det O-9®, and Olive oil.

Another embodiment comprises cyclosporin A, Tegin®, and Olive oil.

Another embodiment comprises cyclosporin A, Tergitol 15-S-12®, and Olive oil.

Another embodiment comprises cyclosporin A, Tergitol 15-S-15®, and Olive oil.

Another embodiment comprises cyclosporin A, Tergitol 15-S-20®, and Olive oil.

Another embodiment comprises cyclosporin A, Tergitol 15-S-20 (80AQ)®, and Olive oil.

Another embodiment comprises cyclosporin A, Tergitol 15-S-3®, and Olive oil.

Another embodiment comprises cyclosporin A, Tergitol 15-S-30®, and Olive oil.

Another embodiment comprises cyclosporin A, Tergitol 15-S-40®, and Olive oil.

Another embodiment comprises cyclosporin A, Tergitol 15-S-40 (70AQ)®, and Olive oil.

Another embodiment comprises cyclosporin A, Tergitol 15-S-5®, and Olive oil.

Another embodiment comprises cyclosporin A, Tergitol 15-S-7®, and Olive oil.

Another embodiment comprises cyclosporin A, Tergitol 15-S-9®, and Olive oil.

Another embodiment comprises cyclosporin A, Tergitol L-101®, and Olive oil.

Another embodiment comprises cyclosporin A, Tergitol L-61®, and Olive oil.

Another embodiment comprises cyclosporin A, Tergitol L-62®, and Olive oil.

Another embodiment comprises cyclosporin A, Tergitol L-64®, and Olive oil.

Another embodiment comprises cyclosporin A, Tergitol L-81®, and Olive oil.

Another embodiment comprises cyclosporin A, Tergitol NP-10®, and Olive oil.

Another embodiment comprises cyclosporin A, Tergitol NP-11®, and Olive oil.

Another embodiment comprises cyclosporin A, Tergitol NP-12®, and Olive oil.

Another embodiment comprises cyclosporin A, Tergitol NP-13®, and Olive oil.

Another embodiment comprises cyclosporin A, Tergitol NP-15®, and Olive oil.

Another embodiment comprises cyclosporin A, Tergitol NP-30®, and Olive oil.

Another embodiment comprises cyclosporin A, Tergitol NP-30 (70 AQ)®, and Olive oil.

Another embodiment comprises cyclosporin A, Tergitol NP-4®, and Olive oil.

Another embodiment comprises cyclosporin A, Tergitol NP-40®, and Olive oil.

Another embodiment comprises cyclosporin A, Tergitol NP-40 (70 AQ)®, and Olive oil.

Another embodiment comprises cyclosporin A, Tergitol NP-50 (70 AQ)®, and Olive oil.

Another embodiment comprises cyclosporin A, Tergitol NP-55 (70 AQ)®, and Olive oil.

Another embodiment comprises cyclosporin A, Tergitol NP-6®, and Olive oil.

Another embodiment comprises cyclosporin A, Tergitol NP-7®, and Olive oil.

Another embodiment comprises cyclosporin A, Tergitol NP-70 (70 AQ)®, and Olive oil.

Another embodiment comprises cyclosporin A, Tergitol NP-8®, and Olive oil.

Another embodiment comprises cyclosporin A, Tergitol NP-9®, and Olive oil.

Another embodiment comprises cyclosporin A, Tergitol NP-9.5®, and Olive oil.

Another embodiment comprises cyclosporin A, Tergitol TMN-10®, and Olive oil.

Another embodiment comprises cyclosporin A, Tergitol TMN-3®, and Olive oil.

Another embodiment comprises cyclosporin A, Tergitol TMN-6®, and Olive oil.

Another embodiment comprises cyclosporin A, Tergitol XD®, and Olive oil.

Another embodiment comprises cyclosporin A, Tergitol XH®, and Olive oil.

Another embodiment comprises cyclosporin A, Tergitol XJ®, and Olive oil.

Another embodiment comprises cyclosporin A, Tetronic 1107®, and Olive oil.

Another embodiment comprises cyclosporin A, Tetronic 1307®, and Olive oil.

Another embodiment comprises cyclosporin A, Tetronic 150R1®, and Olive oil.

Another embodiment comprises cyclosporin A, Tetronic 304®, and Olive oil.

Another embodiment comprises cyclosporin A, Tetronic 701®, and Olive oil.

Another embodiment comprises cyclosporin A, Tetronic 901®, and Olive oil.

Another embodiment comprises cyclosporin A, Tetronic 904®, and Olive oil.

Another embodiment comprises cyclosporin A, Tetronic 908®, and Olive oil.

Another embodiment comprises cyclosporin A, Tetronic 90R4®, and Olive oil.

Another embodiment comprises cyclosporin A, Tomadol 1-3®, and Olive oil.

Another embodiment comprises cyclosporin A, Tomadol 1-5®, and Olive oil.

Another embodiment comprises cyclosporin A, Tomadol 1-7®, and Olive oil.

Another embodiment comprises cyclosporin A, Tomadol 23-1®, and Olive oil.

Another embodiment comprises cyclosporin A, Tomadol 23-3®, and Olive oil.

Another embodiment comprises cyclosporin A, Tomadol 23-5®, and Olive oil.

Another embodiment comprises cyclosporin A, Tomadol 23-6.5®, and Olive oil.

Another embodiment comprises cyclosporin A, Tomadol 25-12®, and Olive oil.

Another embodiment comprises cyclosporin A, Tomadol 25-3®, and Olive oil.

Another embodiment comprises cyclosporin A, Tomadol 25-7®, and Olive oil.

Another embodiment comprises cyclosporin A, Tomadol 25-9®, and Olive oil.

Another embodiment comprises cyclosporin A, Tomadol 45-13®, and Olive oil.

Another embodiment comprises cyclosporin A, Tomadol 45-2.5®, and Olive oil.

Another embodiment comprises cyclosporin A, Tomadol 45-7®, and Olive oil.

Another embodiment comprises cyclosporin A, Tomadol 91-2.5®, and Olive oil.

Another embodiment comprises cyclosporin A, Tomadol 91-6®, and Olive oil.

Another embodiment comprises cyclosporin A, Tomadol 91-8®, and Olive oil.

Another embodiment comprises cyclosporin A, Triton X-100®, and Olive oil.

Another embodiment comprises cyclosporin A, Triton X-102®, and Olive oil.

Another embodiment comprises cyclosporin A, Triton X-114®, and Olive oil.

Another embodiment comprises cyclosporin A, Triton X-15®, and Olive oil.

Another embodiment comprises cyclosporin A, Triton X-165 (70%)®, and Olive oil.

Another embodiment comprises cyclosporin A, Triton X-207®, and Olive oil.

Another embodiment comprises cyclosporin A, Triton X-305 (70%)®, and Olive oil.

Another embodiment comprises cyclosporin A, Triton X-35®, and Olive oil.

Another embodiment comprises cyclosporin A, Triton X-405 (70%)®, and Olive oil.

Another embodiment comprises cyclosporin A, Triton X-45®, and Olive oil.

Another embodiment comprises cyclosporin A, Triton X-705 (70%)®, and Olive oil.

Another embodiment comprises cyclosporin A, Volpo S-10®, and Olive oil.

Another embodiment comprises cyclosporin A, Volpo S-2®, and Olive oil.

Another embodiment comprises cyclosporin A, Volpo S-20®, and Olive oil.

Another embodiment comprises cyclosporin A, Volpo-10®, and Olive oil.

Another embodiment comprises cyclosporin A, Volpo-20®, and Olive oil.

Another embodiment comprises cyclosporin A, Volpo-3®, and Olive oil.

Another embodiment comprises cyclosporin A, Volpo-5®, and Olive oil.

Another embodiment comprises cyclosporin A, Abex 2515®, and Caraway oil.

Another embodiment comprises cyclosporin A, Abex 2525/40®, and Caraway oil.

Another embodiment comprises cyclosporin A, Abex 2535®, and Caraway oil.

Another embodiment comprises cyclosporin A, Abex 2545®, and Caraway oil.

Another embodiment comprises cyclosporin A, Acconon E®, and Caraway oil.

Another embodiment comprises cyclosporin A, Alfonic 1216CO-9®, and Caraway oil.

Another embodiment comprises cyclosporin A, Alionic 1412-3®, and Caraway oil.

Another embodiment comprises cyclosporin A, Alfonic TDA-12®, and Caraway oil.

Another embodiment comprises cyclosporin A, Alfonic TDA-3®, and Caraway oil.

Another embodiment comprises cyclosporin A, Alfonic TDA-4®, and Caraway oil.

Another embodiment comprises cyclosporin A, Alfonic TDA-6®, and Caraway oil.

Another embodiment comprises cyclosporin A, Alfonic TDA-7®, and Caraway oil.

Another embodiment comprises cyclosporin A, Alfonic TDA-8®, and Caraway oil.

Another embodiment comprises cyclosporin A, Alfonic TDA-9®, and Caraway oil.

Another embodiment comprises cyclosporin A, Alkamuls 400-DO®, and Caraway oil.

Another embodiment comprises cyclosporin A, Alkamuls 400-MO/E®, and Caraway oil.

Another embodiment comprises cyclosporin A, Ameroxol OE-10®, and Caraway oil.

Another embodiment comprises cyclosporin A, Ameroxol OE-2®, and Caraway oil.

Another embodiment comprises cyclosporin A, Ameroxol OE-20®, and Caraway oil.

Another embodiment comprises cyclosporin A, Armix 176®, and Caraway oil.

Another embodiment comprises cyclosporin A, Armix 180-C®, and Caraway oil.

Another embodiment comprises cyclosporin A, Armix 183®, and Caraway oil.

Another embodiment comprises cyclosporin A, Brij 35®, and Caraway oil.

Another embodiment comprises cyclosporin A, Brij 35 Liquid®, and Caraway oil.

Another embodiment comprises cyclosporin A, Brij 35 SP®, and Caraway oil.

Another embodiment comprises cyclosporin A, Canasol MJ 2109®, and Caraway oil.

Another embodiment comprises cyclosporin A, Canasol MJ 52®, and Caraway oil.

Another embodiment comprises cyclosporin A, Canasol R 3600®, and Caraway oil.

Another embodiment comprises cyclosporin A, Canasol R 3603®, and Caraway oil.

Another embodiment comprises cyclosporin A, Canasol R 4000 H®, and Caraway oil.

Another embodiment comprises cyclosporin A, Cedepal CA-210®, and Caraway oil.

Another embodiment comprises cyclosporin A, Cedepal CA-520®, and Caraway oil.

Another embodiment comprises cyclosporin A, Cedepal CA-630®, and Caraway oil.

Another embodiment comprises cyclosporin A, Cedepal CA-720®, and Caraway oil.

Another embodiment comprises cyclosporin A, Cedepal CA-890®, and Caraway oil.

Another embodiment comprises cyclosporin A, Cedepal CA-897®, and Caraway oil.

Another embodiment comprises cyclosporin A, Cedepal CO-210®, and Caraway oil.

Another embodiment comprises cyclosporin A, Cedepal CO-430®, and Caraway oil.

Another embodiment comprises cyclosporin A, Cedepal CO-530®, and Caraway oil.

Another embodiment comprises cyclosporin A, Cedepal CO-630®, and Caraway oil.

Another embodiment comprises cyclosporin A, Cedepal CO-710®, and Caraway oil.

Another embodiment comprises cyclosporin A, Cedepal CO-730®, and Caraway oil.

Another embodiment comprises cyclosporin A, Cedepal CO-880®, and Caraway oil.

Another embodiment comprises cyclosporin A, Cedepal CO-877®, and Caraway oil.

Another embodiment comprises cyclosporin A, Cedepal CO-890®, and Caraway oil.

Another embodiment comprises cyclosporin A, Cedepal CO-897®, and Caraway oil.

Another embodiment comprises cyclosporin A, Cedepal CO-970®, and Caraway oil.

Another embodiment comprises cyclosporin A, Cedepal CO-977®, and Caraway oil.

Another embodiment comprises cyclosporin A, Cedepal CO-990®, and Caraway oil.

Another embodiment comprises cyclosporin A, Cedepal CO-997®, and Caraway oil.

Another embodiment comprises cyclosporin A, Chemal BP 261®, and Caraway oil.

Another embodiment comprises cyclosporin A, Chemal BP 262®, and Caraway oil.

Another embodiment comprises cyclosporin A, Chemal BP 3172®, and Caraway oil.

Another embodiment comprises cyclosporin A, Chemal BP 3174®, and Caraway oil.

Another embodiment comprises cyclosporin A, Chemal DA-4®, and Caraway oil.

Another embodiment comprises cyclosporin A, Chemal DA-6®, and Caraway oil.

Another embodiment comprises cyclosporin A, Chemal DA-9®, and Caraway oil.

Another embodiment comprises cyclosporin A, Chemal LA-23®, and Caraway oil.

Another embodiment comprises cyclosporin A, Chemal LA-4®, and Caraway oil.

Another embodiment comprises cyclosporin A, Chemal LA-9®, and Caraway oil.

Another embodiment comprises cyclosporin A, Chemal OA-20G®, and Caraway oil.

Another embodiment comprises cyclosporin A, Chemal OA-5®, and Caraway oil.

Another embodiment comprises cyclosporin A, Chemal OA-9®, and Caraway oil.

Another embodiment comprises cyclosporin A, Chemal TDA-12®, and Caraway oil.

Another embodiment comprises cyclosporin A, Chemal TDA-15®, and Caraway oil.

Another embodiment comprises cyclosporin A, Chemal TDA-3®, and Caraway oil.

Another embodiment comprises cyclosporin A, Chemal TDA-6®, and Caraway oil.

Another embodiment comprises cyclosporin A, Chemal TDA-9®, and Caraway oil.

Another embodiment comprises cyclosporin A, Chemax CO-16®, and Caraway oil.

Another embodiment comprises cyclosporin A, Chemax CO-200/50®, and Caraway oil.

Another embodiment comprises cyclosporin A, Chemax CO-25®, and Caraway oil.

Another embodiment comprises cyclosporin A, Chemax CO-30®, and Caraway oil.

Another embodiment comprises cyclosporin A, Chemax CO-36®, and Caraway oil.

Another embodiment comprises cyclosporin A, Chemax CO-40®, and Caraway oil.

Another embodiment comprises cyclosporin A, Chemax CO-5®, and Caraway oil.

Another embodiment comprises cyclosporin A, Chemax CO-80®, and Caraway oil.

Another embodiment comprises cyclosporin A, Chemax DNP-150/50®, and Caraway oil.

Another embodiment comprises cyclosporin A, Chemax DNP-18®, and Caraway oil.

Another embodiment comprises cyclosporin A, Chemax DNP-8®, and Caraway oil.

Another embodiment comprises cyclosporin A, Chemax E 200 ML®, and Caraway oil.

Another embodiment comprises cyclosporin A, Chemax E 200 MO®, and Caraway oil.

Another embodiment comprises cyclosporin A, Chemax E 400 ML®, and Caraway oil.

Another embodiment comprises cyclosporin A, Chemax E 400 MO®, and Caraway oil.

Another embodiment comprises cyclosporin A, Chemax E 600 ML®, and Caraway oil.

Another embodiment comprises cyclosporin A, Chemax E 600 MO®, and Caraway oil.

Another embodiment comprises cyclosporin A, Chemax E1000 MO®, and Caraway oil.

Another embodiment comprises cyclosporin A, Chemax E1000 MS®, and Caraway oil.

Another embodiment comprises cyclosporin A, Chemax E200 MS®, and Caraway oil.

Another embodiment comprises cyclosporin A, Chemax E400 MS®, and Caraway oil.

Another embodiment comprises cyclosporin A, Chemax E600 MS®, and Caraway oil.

Another embodiment comprises cyclosporin A, Chemax HCO-16®, and Caraway oil.

Another embodiment comprises cyclosporin A, Chemax HCO-200/50®, and Caraway oil.

Another embodiment comprises cyclosporin A, Chemax HCO-25®, and Caraway oil.

Another embodiment comprises cyclosporin A, Chemax HCO-5®, and Caraway oil.

Another embodiment comprises cyclosporin A, Chemax TO-16®, and Caraway oil.

Another embodiment comprises cyclosporin A, Chemax TO-8®, and Caraway oil.

Another embodiment comprises cyclosporin A, Colalube 3409®, and Caraway oil.

Another embodiment comprises cyclosporin A, Colalube 3411®, and Caraway oil.

Another embodiment comprises cyclosporin A, Colalube 3413®, and Caraway oil.

Another embodiment comprises cyclosporin A, Colalube 3414®, and Caraway oil.

Another embodiment comprises cyclosporin A, Colalube 3415®, and Caraway oil.

Another embodiment comprises cyclosporin A, Colalube 3416®, and Caraway oil.

Another embodiment comprises cyclosporin A, Colalube 3417®, and Caraway oil.

Another embodiment comprises cyclosporin A, Delonic NPE-100®, and Caraway oil.

Another embodiment comprises cyclosporin A, Delonic NPE-30®, and Caraway oil.

Another embodiment comprises cyclosporin A, Delonic NPE-40®, and Caraway oil.

Another embodiment comprises cyclosporin A, Delonic OPE-10®, and Caraway oil.

Another embodiment comprises cyclosporin A, Delonic OPE-12®, and Caraway oil.

Another embodiment comprises cyclosporin A, Delonic OPE-30®, and Caraway oil.

Another embodiment comprises cyclosporin A, Delonic OPE-40®, and Caraway oil.

Another embodiment comprises cyclosporin A, Delonic OPE-5®, and Caraway oil.

Another embodiment comprises cyclosporin A, Delonic OPE-7.5®, and Caraway oil.

Another embodiment comprises cyclosporin A, DePeg 16-CO®, and Caraway oil.

Another embodiment comprises cyclosporin A, DePeg 25-CO®, and Caraway oil.

Another embodiment comprises cyclosporin A, DePeg 30-CO®, and Caraway oil.

Another embodiment comprises cyclosporin A, DePeg 40-CO®, and Caraway oil.

Another embodiment comprises cyclosporin A, DePeg 5-CO®, and Caraway oil.

Another embodiment comprises cyclosporin A, DePeg 80-CO®, and Caraway oil.

Another embodiment comprises cyclosporin A, DeSonic S-100®, and Caraway oil.

Another embodiment comprises cyclosporin A, DeSonic S-114®, and Caraway oil.

Another embodiment comprises cyclosporin A, DeSonic S-405®, and Caraway oil.

Another embodiment comprises cyclosporin A, DeSonic S-45®, and Caraway oil.

Another embodiment comprises cyclosporin A, DeSonic TDA-9®, and Caraway oil.

Another embodiment comprises cyclosporin A, DeThox Acid O-14®, and Caraway oil.

Another embodiment comprises cyclosporin A, DeThox Acid O-16®, and Caraway oil.

Another embodiment comprises cyclosporin A, DeThox Acid O-9®, and Caraway oil.

Another embodiment comprises cyclosporin A, DeThox Acid TO-14®, and Caraway oil.

Another embodiment comprises cyclosporin A, DeThox Acid TO-16.5®, and Caraway oil.

Another embodiment comprises cyclosporin A, DeThox Acid TO-8.5®, and Caraway oil.

Another embodiment comprises cyclosporin A, Dur-Em 114®, and Caraway oil.

Another embodiment comprises cyclosporin A, Dur-Em 117®, and Caraway oil.

Another embodiment comprises cyclosporin A, Dur-Em 204®, and Caraway oil.

Another embodiment comprises cyclosporin A, Dur-Em 207®, and Caraway oil.

Another embodiment comprises cyclosporin A, Dur-Em 207E®, and Caraway oil.

Another embodiment comprises cyclosporin A, Emerest 2704®, and Caraway oil.

Another embodiment comprises cyclosporin A, Emerest 2712®, and Caraway oil.

Another embodiment comprises cyclosporin A, Emerest 2715®, and Caraway oil.

Another embodiment comprises cyclosporin A, Ethal EH-2®, and Caraway oil.

Another embodiment comprises cyclosporin A, Ethal EH-5®, and Caraway oil.

Another embodiment comprises cyclosporin A, Ethal OA-23®, and Caraway oil.

Another embodiment comprises cyclosporin A, Ethosperse CA-20®, and Caraway oil.

Another embodiment comprises cyclosporin A, Ethosperse LA-12®, and Caraway oil.

Another embodiment comprises cyclosporin A, Ethosperse LA-23®, and Caraway oil.

Another embodiment comprises cyclosporin A, Ethosperse LA-4®, and Caraway oil.

Another embodiment comprises cyclosporin A, Ethox 3095®, and Caraway oil.

Another embodiment comprises cyclosporin A, Ethox CO-16®, and Caraway oil.

Another embodiment comprises cyclosporin A, Ethox CO-200®, and Caraway oil.

Another embodiment comprises cyclosporin A, Ethox CO-25®, and Caraway oil.

Another embodiment comprises cyclosporin A, Ethox CO-30®, and Caraway oil.

Another embodiment comprises cyclosporin A, Ethox CO-36®, and Caraway oil.

Another embodiment comprises cyclosporin A, Ethox CO-40®, and Caraway oil.

Another embodiment comprises cyclosporin A, Ethox CO-5®, and Caraway oil.

Another embodiment comprises cyclosporin A, Ethox DO-14®, and Caraway oil.

Another embodiment comprises cyclosporin A, Ethox DO-9®, and Caraway oil.

Another embodiment comprises cyclosporin A, Ethox MA-8®, and Caraway oil.

Another embodiment comprises cyclosporin A, Ethox MI-14®, and Caraway oil.

Another embodiment comprises cyclosporin A, Ethox ML-14®, and Caraway oil.

Another embodiment comprises cyclosporin A, Ethox ML-5®, and Caraway oil.

Another embodiment comprises cyclosporin A, Ethox MO-14®, and Caraway oil.

Another embodiment comprises cyclosporin A, Ethox MO-9®, and Caraway oil.

Another embodiment comprises cyclosporin A, Ethox MS-14®, and Caraway oil.

Another embodiment comprises cyclosporin A, Ethox MS-23®, and Caraway oil.

Another embodiment comprises cyclosporin A, Ethox MS-40®, and Caraway oil.

Another embodiment comprises cyclosporin A, Ethox MS-8®, and Caraway oil.

Another embodiment comprises cyclosporin A, Ethox TO-16®, and Caraway oil.

Another embodiment comprises cyclosporin A, Ethox TO-8®, and Caraway oil.

Another embodiment comprises cyclosporin A, Ethylan SN-120®, and Caraway oil.

Another embodiment comprises cyclosporin A, Ethylan SN-70®, and Caraway oil.

Another embodiment comprises cyclosporin A, Ethylan SN-90®, and Caraway oil.

Another embodiment comprises cyclosporin A, Flexricin 13®, and Caraway oil.

Another embodiment comprises cyclosporin A, Flexricin 15®, and Caraway oil.

Another embodiment comprises cyclosporin A, Genapol 1261®, and Caraway oil.

Another embodiment comprises cyclosporin A, Generol 122 E25®, and Caraway oil.

Another embodiment comprises cyclosporin A, Glicepol 160®, and Caraway oil.

Another embodiment comprises cyclosporin A, Glicepol 560®, and Caraway oil.

Another embodiment comprises cyclosporin A, Glicepol GMS 20®, and Caraway oil.

Another embodiment comprises cyclosporin A, Hetoxide BN-13®, and Caraway oil.

Another embodiment comprises cyclosporin A, Hetoxide DNP-4®, and Caraway oil.

Another embodiment comprises cyclosporin A, Hetoxide DNP-9.6®, and Caraway oil.

Another embodiment comprises cyclosporin A, Hetoxide G-26®, and Caraway oil.

Another embodiment comprises cyclosporin A, Hetoxide G-7®, and Caraway oil.

Another embodiment comprises cyclosporin A, Hetoxide GT-20®, and Caraway oil.

Another embodiment comprises cyclosporin A, Hetoxide HC-40®, and Caraway oil.

Another embodiment comprises cyclosporin A, Hetoxol C-24®, and Caraway oil.

Another embodiment comprises cyclosporin A, Hetoxol CA-10®, and Caraway oil.

Another embodiment comprises cyclosporin A, Hetoxol CA-2®, and Caraway oil.

Another embodiment comprises cyclosporin A, Hetoxol CA-20®, and Caraway oil.

Another embodiment comprises cyclosporin A, Hetoxol CAWS®, and Caraway oil.

Another embodiment comprises cyclosporin A, Hetoxol CD-3®, and Caraway oil.

Another embodiment comprises cyclosporin A, Hetoxol CD-4®, and Caraway oil.

Another embodiment comprises cyclosporin A, Hetoxol CS-15®, and Caraway oil.

Another embodiment comprises cyclosporin A, Hetoxol CS-20®, and Caraway oil.

Another embodiment comprises cyclosporin A, Hetoxol CS-20D®, and Caraway oil.

Another embodiment comprises cyclosporin A, Hetoxol CS-25®, and Caraway oil.

Another embodiment comprises cyclosporin A, Hetoxol CS-50®, and Caraway oil.

Another embodiment comprises cyclosporin A, Hetoxol CS-9®, and Caraway oil.

Another embodiment comprises cyclosporin A, Hetoxol CSA-15®, and Caraway oil.

Another embodiment comprises cyclosporin A, Hetoxol D®, and Caraway oil.

Another embodiment comprises cyclosporin A, Hetoxol G®, and Caraway oil.

Another embodiment comprises cyclosporin A, Hetoxol J®, and Caraway oil.

Another embodiment comprises cyclosporin A, Hetoxol L®, and Caraway oil.

Another embodiment comprises cyclosporin A, Hetoxol L-23®, and Caraway oil.

Another embodiment comprises cyclosporin A, Hetoxol L-4®, and Caraway oil.

Another embodiment comprises cyclosporin A, Hetoxol L-9®, and Caraway oil.

Another embodiment comprises cyclosporin A, Hetoxol LS-9®, and Caraway oil.

Another embodiment comprises cyclosporin A, Hetoxol M-3®, and Caraway oil.

Another embodiment comprises cyclosporin A, Hetoxol OA-10 Special®, and Caraway oil.

Another embodiment comprises cyclosporin A, Hetoxol OA-20 Special®, and Caraway oil.

Another embodiment comprises cyclosporin A, Hetoxol OA-3 Special®, and Caraway oil.

Another embodiment comprises cyclosporin A, Hetoxol OA-5 Special®, and Caraway oil.

Another embodiment comprises cyclosporin A, Hetoxol OL-10®, and Caraway oil.

Another embodiment comprises cyclosporin A, Hetoxol OL-2®, and Caraway oil.

Another embodiment comprises cyclosporin A, Hetoxol OL-23®, and Caraway oil.

Another embodiment comprises cyclosporin A, Hetoxol OL-4®, and Caraway oil.

Another embodiment comprises cyclosporin A, Hetoxol OL-40®, and Caraway oil.

Another embodiment comprises cyclosporin A, Hetoxol OL-5®, and Caraway oil.

Another embodiment comprises cyclosporin A, Hetoxol PLA®, and Caraway oil.

Another embodiment comprises cyclosporin A, Hetoxol SP-15®, and Caraway oil.

Another embodiment comprises cyclosporin A, Hetoxol STA-10®, and Caraway oil.

Another embodiment comprises cyclosporin A, Hetoxol STA-2®, and Caraway oil.

Another embodiment comprises cyclosporin A, Hetoxol STA-30®, and Caraway oil.

Another embodiment comprises cyclosporin A, Hetoxol TD-12®, and Caraway oil.

Another embodiment comprises cyclosporin A, Hetoxol TD-18®, and Caraway oil.

Another embodiment comprises cyclosporin A, Hetoxol TD-3®, and Caraway oil.

Another embodiment comprises cyclosporin A, Hetoxol TD-6®, and Caraway oil.

Another embodiment comprises cyclosporin A, Hetoxol TD-9®, and Caraway oil.

Another embodiment comprises cyclosporin A, Hetoxol TDEP-15®, and Caraway oil.

Another embodiment comprises cyclosporin A, Hetoxol TDEP-63®, and Caraway oil.

Another embodiment comprises cyclosporin A, Iconol 24-12®, and Caraway oil.

Another embodiment comprises cyclosporin A, Iconol 24-23®, and Caraway oil.

Another embodiment comprises cyclosporin A, Iconol 24-4®, and Caraway oil.

Another embodiment comprises cyclosporin A, Iconol DA-4®, and Caraway oil.

Another embodiment comprises cyclosporin A, Iconol DA-6®, and Caraway oil.

Another embodiment comprises cyclosporin A, Iconol TDA-10®, and Caraway oil.

Another embodiment comprises cyclosporin A, Iconol TDA-3®, and Caraway oil.

Another embodiment comprises cyclosporin A, Iconol TDA-6®, and Caraway oil.

Another embodiment comprises cyclosporin A, Iconol TDA-8-90%®, and Caraway oil.

Another embodiment comprises cyclosporin A, Iconol TDA-9®, and Caraway oil.

Another embodiment comprises cyclosporin A, Igepal CA-210®, and Caraway oil.

Another embodiment comprises cyclosporin A, Igepal CA-407®, and Caraway oil.

Another embodiment comprises cyclosporin A, Igepal CA-420®, and Caraway oil.

Another embodiment comprises cyclosporin A, Igepal CA-520®, and Caraway oil.

Another embodiment comprises cyclosporin A, Igepal CA-620®, and Caraway oil.

Another embodiment comprises cyclosporin A, Igepal CA-630®, and Caraway oil.

Another embodiment comprises cyclosporin A, Igepal CA-720®, and Caraway oil.

Another embodiment comprises cyclosporin A, Igepal CA-877®, and Caraway oil.

Another embodiment comprises cyclosporin A, Igepal CA-887®, and Caraway oil.

Another embodiment comprises cyclosporin A, Igepal CA-897®, and Caraway oil.

Another embodiment comprises cyclosporin A, Igepal CO-210®, and Caraway oil.

Another embodiment comprises cyclosporin A, Igepal CO-430®, and Caraway oil.

Another embodiment comprises cyclosporin A, Igepal CO-520®, and Caraway oil.

Another embodiment comprises cyclosporin A, Igepal CO-530®, and Caraway oil.

Another embodiment comprises cyclosporin A, Igepal CO-610®, and Caraway oil.

Another embodiment comprises cyclosporin A, Igepal CO-630®, and Caraway oil.

Another embodiment comprises cyclosporin A, Igepal CO-630 Special®, and Caraway oil.

Another embodiment comprises cyclosporin A, Igepal CO-660®, and Caraway oil.

Another embodiment comprises cyclosporin A, Igepal CO-710®, and Caraway oil.

Another embodiment comprises cyclosporin A, Igepal CO-720®, and Caraway oil.

Another embodiment comprises cyclosporin A, Igepal CO-730®, and Caraway oil.

Another embodiment comprises cyclosporin A, Igepal CO-738®, and Caraway oil.

Another embodiment comprises cyclosporin A, Igepal CO-850®, and Caraway oil.

Another embodiment comprises cyclosporin A, Igepal CO-880®, and Caraway oil.

Another embodiment comprises cyclosporin A, Igepal CO-887®, and Caraway oil.

Another embodiment comprises cyclosporin A, Igepal CO-890®, and Caraway oil.

Another embodiment comprises cyclosporin A, Igepal CO-897®, and Caraway oil.

Another embodiment comprises cyclosporin A, Igepal CO-970®, and Caraway oil.

Another embodiment comprises cyclosporin A, Igepal CO-977®, and Caraway oil.

Another embodiment comprises cyclosporin A, Igepal CO-987®, and Caraway oil.

Another embodiment comprises cyclosporin A, Igepal CO-990 FLK®, and Caraway oil.

Another embodiment comprises cyclosporin A, Igepal CO-997®, and Caraway oil.

Another embodiment comprises cyclosporin A, Igepal DM-430®, and Caraway oil.

Another embodiment comprises cyclosporin A, Igepal DM-530®, and Caraway oil.

Another embodiment comprises cyclosporin A, Igepal DM-710®, and Caraway oil.

Another embodiment comprises cyclosporin A, Igepal DM-730®, and Caraway oil.

Another embodiment comprises cyclosporin A, Igepal DM-880®, and Caraway oil.

Another embodiment comprises cyclosporin A, Igepal DM-970 FLK®, and Caraway oil.

Another embodiment comprises cyclosporin A, Igepal OD-410®, and Caraway oil.

Another embodiment comprises cyclosporin A, Igepal RC-520®, and Caraway oil.

Another embodiment comprises cyclosporin A, Igepal RC-620®, and Caraway oil.

Another embodiment comprises cyclosporin A, Igepal RC-630®, and Caraway oil.

Another embodiment comprises cyclosporin A, Lexemul CS-20®, and Caraway oil.

Another embodiment comprises cyclosporin A, Lipo GMS 450®, and Caraway oil.

Another embodiment comprises cyclosporin A, Lipo Polyglycol 1000®, and Caraway oil.

Another embodiment comprises cyclosporin A, Lipo Polyglycol 200®, and Caraway oil.

Another embodiment comprises cyclosporin A, Lipo Polyglycol 300®, and Caraway oil.

Another embodiment comprises cyclosporin A, Lipo Polyglycol 3350®, and Caraway oil.

Another embodiment comprises cyclosporin A, Lipo Polyglycol 400®, and Caraway oil.

Another embodiment comprises cyclosporin A, Lipo Polyglycol 600®, and Caraway oil.

Another embodiment comprises cyclosporin A, Lipocol C-10®, and Caraway oil.

Another embodiment comprises cyclosporin A, Lipocol C-2®, and Caraway oil.

Another embodiment comprises cyclosporin A, Lipocol C-20®, and Caraway oil.

Another embodiment comprises cyclosporin A, Lipocol L-12®, and Caraway oil.

Another embodiment comprises cyclosporin A, Lipocol L-23®, and Caraway oil.

Another embodiment comprises cyclosporin A, Lipocol L-4®, and Caraway oil.

Another embodiment comprises cyclosporin A, Lipocol O-10®, and Caraway oil.

Another embodiment comprises cyclosporin A, Lipocol O-2®, and Caraway oil.

Another embodiment comprises cyclosporin A, Lipocol O-20®, and Caraway oil.

Another embodiment comprises cyclosporin A, Lipocol O-5®, and Caraway oil.

Another embodiment comprises cyclosporin A, Lipocol S-10®, and Caraway oil.

Another embodiment comprises cyclosporin A, Lipocol S-2®, and Caraway oil.

Another embodiment comprises cyclosporin A, Lipocol S-20®, and Caraway oil.

Another embodiment comprises cyclosporin A, Lipocol SC-15®, and Caraway oil.

Another embodiment comprises cyclosporin A, Lipocol SC-20®, and Caraway oil.

Another embodiment comprises cyclosporin A, Lipocol SC-4®, and Caraway oil.

Another embodiment comprises cyclosporin A, Lipocol TD-12®, and Caraway oil.

Another embodiment comprises cyclosporin A, Lumulse 40-O K®, and Caraway oil.

Another embodiment comprises cyclosporin A, Lumulse 42-OK®, and Caraway oil.

Another embodiment comprises cyclosporin A, Lumulse GMT-40®, and Caraway oil.

Another embodiment comprises cyclosporin A, Lumulse PEG 1450®, and Caraway oil.

Another embodiment comprises cyclosporin A, Lumulse PEG 1450 NF®, and Caraway oil.

Another embodiment comprises cyclosporin A, Lumulse PEG 8000®, and Caraway oil.

Another embodiment comprises cyclosporin A, Lumulse PGO®, and Caraway oil.

Another embodiment comprises cyclosporin A, Lumulse POE (2) Oleyl Amine®, and Caraway oil.

Another embodiment comprises cyclosporin A, Magrabar PGO-104®, and Caraway oil.

Another embodiment comprises cyclosporin A, Mapeg 200 ML®, and Caraway oil.

Another embodiment comprises cyclosporin A, Mapeg 400 MO®, and Caraway oil.

Another embodiment comprises cyclosporin A, Mapeg 400 MOT®, and Caraway oil.

Another embodiment comprises cyclosporin A, Mapeg 400DO®, and Caraway oil.

Another embodiment comprises cyclosporin A, Mapeg 400DOT®, and Caraway oil.

Another embodiment comprises cyclosporin A, Mapeg 600DOT®, and Caraway oil.

Another embodiment comprises cyclosporin A, Marlox B 24/80®, and Caraway oil.

Another embodiment comprises cyclosporin A, Marlox FK 86®, and Caraway oil.

Another embodiment comprises cyclosporin A, Marlox MO 154®, and Caraway oil.

Another embodiment comprises cyclosporin A, Mazox CDA CG®, and Caraway oil.

Another embodiment comprises cyclosporin A, Monoglycerides Glyceryl Monestearate®, and Caraway oil.

Another embodiment comprises cyclosporin A, Myrj 45®, and Caraway oil.

Another embodiment comprises cyclosporin A, Myrj 52®, and Caraway oil.

Another embodiment comprises cyclosporin A, Myrj 52S®, and Caraway oil.

Another embodiment comprises cyclosporin A, Myrj 53®, and Caraway oil.

Another embodiment comprises cyclosporin A, Myrj 59®, and Caraway oil.

Another embodiment comprises cyclosporin A, Norfox NP-12®, and Caraway oil.

Another embodiment comprises cyclosporin A, Norfox NP-4®, and Caraway oil.

Another embodiment comprises cyclosporin A, Norfox NP-6®, and Caraway oil.

Another embodiment comprises cyclosporin A, Norfox NP-887®, and Caraway oil.

Another embodiment comprises cyclosporin A, Norfox NP-9®, and Caraway oil.

Another embodiment comprises cyclosporin A, Norfox NP-977®, and Caraway oil.

Another embodiment comprises cyclosporin A, Norfox OP-100®, and Caraway oil.

Another embodiment comprises cyclosporin A, Norfox OP-102®, and Caraway oil.

Another embodiment comprises cyclosporin A, Norfox OP-114®, and Caraway oil.

Another embodiment comprises cyclosporin A, Norfox OP-45®, and Caraway oil.

Another embodiment comprises cyclosporin A, Pegosperse 100-L®, and Caraway oil.

Another embodiment comprises cyclosporin A, Pegosperse 100-O®, and Caraway oil.

Another embodiment comprises cyclosporin A, Pegosperse 100-S®, and Caraway oil.

Another embodiment comprises cyclosporin A, Pegosperse 1500-MS®, and Caraway oil.

Another embodiment comprises cyclosporin A, Pegosperse 1750-MS®, and Caraway oil.

Another embodiment comprises cyclosporin A, Pegosperse 200 DL®, and Caraway oil.

Another embodiment comprises cyclosporin A, Pegosperse 200-ML®, and Caraway oil.

Another embodiment comprises cyclosporin A, Pegosperse 400-DL®, and Caraway oil.

Another embodiment comprises cyclosporin A, Pegosperse 400-DO®, and Caraway oil.

Another embodiment comprises cyclosporin A, Pegosperse 400-DS®, and Caraway oil.

Another embodiment comprises cyclosporin A, Pegosperse 400-ML®, and Caraway oil.

Another embodiment comprises cyclosporin A, Pegosperse 400-MO®, and Caraway oil.

Another embodiment comprises cyclosporin A, Pegosperse 400-MOT®, and Caraway oil.

Another embodiment comprises cyclosporin A, Pegosperse 400-MS®, and Caraway oil.

Another embodiment comprises cyclosporin A, Pegosperse 50-DS®, and Caraway oil.

Another embodiment comprises cyclosporin A, Pegosperse 50-MS®, and Caraway oil.

Another embodiment comprises cyclosporin A, Pegosperse 600-ML®, and Caraway oil.

Another embodiment comprises cyclosporin A, Pegosperse 600-MS®, and Caraway oil.

Another embodiment comprises cyclosporin A, Plurafac A-38®, and Caraway oil.

Another embodiment comprises cyclosporin A, Plurafac B-25-5®, and Caraway oil.

Another embodiment comprises cyclosporin A, Plurafac B-26®, and Caraway oil.

Another embodiment comprises cyclosporin A, Plurafac D-25®, and Caraway oil.

Another embodiment comprises cyclosporin A, Plurafac LF 131®, and Caraway oil.

Another embodiment comprises cyclosporin A, Plurafac LF 4030®, and Caraway oil.

Another embodiment comprises cyclosporin A, Plurafac LF 7000®, and Caraway oil.

Another embodiment comprises cyclosporin A, Plurafac RA-20®, and Caraway oil.

Another embodiment comprises cyclosporin A, Plurafac RA-30®, and Caraway oil.

Another embodiment comprises cyclosporin A, Plurafac RA-40®, and Caraway oil.

Another embodiment comprises cyclosporin A, Pluronic 10R5®, and Caraway oil.

Another embodiment comprises cyclosporin A, Pluronic 17R2®, and Caraway oil.

Another embodiment comprises cyclosporin A, Pluronic 17R4®, and Caraway oil.

Another embodiment comprises cyclosporin A, Pluronic 25R2®, and Caraway oil.

Another embodiment comprises cyclosporin A, Pluronic 25R4®, and Caraway oil.

Another embodiment comprises cyclosporin A, Pluronic 31R1®, and Caraway oil.

Another embodiment comprises cyclosporin A, Pluronic F108®, and Caraway oil.

Another embodiment comprises cyclosporin A, Pluronic F127®, and Caraway oil.

Another embodiment comprises cyclosporin A, Pluronic F38®, and Caraway oil.

Another embodiment comprises cyclosporin A, Pluronic F68®, and Caraway oil.

Another embodiment comprises cyclosporin A, Pluronic F68LF®, and Caraway oil.

Another embodiment comprises cyclosporin A, Pluronic F77®, and Caraway oil.

Another embodiment comprises cyclosporin A, Pluronic F87®, and Caraway oil.

Another embodiment comprises cyclosporin A, Pluronic F88®, and Caraway oil.

Another embodiment comprises cyclosporin A, Pluronic F98®, and Caraway oil.

Another embodiment comprises cyclosporin A, Pluronic L10®, and Caraway oil.

Another embodiment comprises cyclosporin A, Pluronic L101®, and Caraway oil.

Another embodiment comprises cyclosporin A, Pluronic L121®, and Caraway oil.

Another embodiment comprises cyclosporin A, Pluronic L31®, and Caraway oil.

Another embodiment comprises cyclosporin A, Pluronic L35®, and Caraway oil.

Another embodiment comprises cyclosporin A, Pluronic L43®, and Caraway oil.

Another embodiment comprises cyclosporin A, Pluronic L44®, and Caraway oil.

Another embodiment comprises cyclosporin A, Pluronic L61®, and Caraway oil.

Another embodiment comprises cyclosporin A, Pluronic L62®, and Caraway oil.

Another embodiment comprises cyclosporin A, Pluronic L62D®, and Caraway oil.

Another embodiment comprises cyclosporin A, Pluronic L62LF®, and Caraway oil.

Another embodiment comprises cyclosporin A, Pluronic L64®, and Caraway oil.

Another embodiment comprises cyclosporin A, Pluronic L81®, and Caraway oil.

Another embodiment comprises cyclosporin A, Pluronic L92®, and Caraway oil.

Another embodiment comprises cyclosporin A, Pluronic P103®, and Caraway oil.

Another embodiment comprises cyclosporin A, Pluronic P104®, and Caraway oil.

Another embodiment comprises cyclosporin A, Pluronic P105®, and Caraway oil.

Another embodiment comprises cyclosporin A, Pluronic P123®, and Caraway oil.

Another embodiment comprises cyclosporin A, Pluronic P65®, and Caraway oil.

Another embodiment comprises cyclosporin A, Pluronic P84®, and Caraway oil.

Another embodiment comprises cyclosporin A, Pluronic P85®, and Caraway oil.

Another embodiment comprises cyclosporin A, Polyaldo 10-2-P®, and Caraway oil.

Another embodiment comprises cyclosporin A, Polyaldo DGDO®, and Caraway oil.

Another embodiment comprises cyclosporin A, Polyaldo HGDS®, and Caraway oil.

Another embodiment comprises cyclosporin A, Polyaldo TGMS®, and Caraway oil.

Another embodiment comprises cyclosporin A, Protegin V®, and Caraway oil.

Another embodiment comprises cyclosporin A, Protegin W®, and Caraway oil.

Another embodiment comprises cyclosporin A, Protegin WX®, and Caraway oil.

Another embodiment comprises cyclosporin A, Protegin XV®, and Caraway oil.

Another embodiment comprises cyclosporin A, Rhodasurf BC-420®, and Caraway oil.

Another embodiment comprises cyclosporin A, Rhodasurf BC-610, and Caraway oil.

Another embodiment comprises cyclosporin A, Rhodasurf BC-630®, and Caraway oil.

Another embodiment comprises cyclosporin A, Rhodasurf BC-720®, and Caraway oil.

Another embodiment comprises cyclosporin A, Rhodasurf BC-840®, and Caraway oil.

Another embodiment comprises cyclosporin A, Rhodasurf L 1®, and Caraway oil.

Another embodiment comprises cyclosporin A, Rhodasurf L 12®, and Caraway oil.

Another embodiment comprises cyclosporin A, Rhodasurf L 2®, and Caraway oil.

Another embodiment comprises cyclosporin A, Rhodasurf L 20®, and Caraway oil.

Another embodiment comprises cyclosporin A, Rhodasurf L 3®, and Caraway oil.

Another embodiment comprises cyclosporin A, Rhodasurf L 30®, and Caraway oil.

Another embodiment comprises cyclosporin A, Rhodasurf L 4®, and Caraway oil.

Another embodiment comprises cyclosporin A, Rhodasurf L 50®, and Caraway oil.

Another embodiment comprises cyclosporin A, Rhodasurf L 7/90®, and Caraway oil.

Another embodiment comprises cyclosporin A, Rhodasurf LA-3®, and Caraway oil.

Another embodiment comprises cyclosporin A, Rhodasurf LA-7®, and Caraway oil.

Another embodiment comprises cyclosporin A, Rhodasurf LAN-23®, and Caraway oil.

Another embodiment comprises cyclosporin A, Rhodasurf ON-870®, and Caraway oil.

Another embodiment comprises cyclosporin A, Rhodasurf ON-877®, and Caraway oil.

Another embodiment comprises cyclosporin A, Rhodasurf TDA-8/5®, and Caraway oil.

Another embodiment comprises cyclosporin A, Ritapro 100®, and Caraway oil.

Another embodiment comprises cyclosporin A, Ritapro 165®, and Caraway oil.

Another embodiment comprises cyclosporin A, Ritapro 200®, and Caraway oil.

Another embodiment comprises cyclosporin A, Ritapro 300®, and Caraway oil.

Another embodiment comprises cyclosporin A, Ritoleth 10®, and Caraway oil.

Another embodiment comprises cyclosporin A, Ritoleth 2®, and Caraway oil.

Another embodiment comprises cyclosporin A, Ritoleth 20®, and Caraway oil.

Another embodiment comprises cyclosporin A, Ritoleth 5®, and Caraway oil.

Another embodiment comprises cyclosporin A, Santone 10-10-0®, and Caraway oil.

Another embodiment comprises cyclosporin A, Santone 3-1-S XTR®, and Caraway oil.

Another embodiment comprises cyclosporin A, Santone 3-1-SH®, and Caraway oil.

Another embodiment comprises cyclosporin A, Santone 8-1-O®, and Caraway oil.

Another embodiment comprises cyclosporin A, Solulan 16®, and Caraway oil.

Another embodiment comprises cyclosporin A, Solulan 75®, and Caraway oil.

Another embodiment comprises cyclosporin A, Solulan C-24®, and Caraway oil.

Another embodiment comprises cyclosporin A, Solulan L-575®, and Caraway oil.

Another embodiment comprises cyclosporin A, Standamox CAW®, and Caraway oil.

Another embodiment comprises cyclosporin A, Surfine AZI-A®, and Caraway oil.

Another embodiment comprises cyclosporin A, Surfonic CO-15®, and Caraway oil.

Another embodiment comprises cyclosporin A, Surfonic CO-25®, and Caraway oil.

Another embodiment comprises cyclosporin A, Surfonic CO-30®, and Caraway oil.

Another embodiment comprises cyclosporin A, Surfonic CO-36®, and Caraway oil.

Another embodiment comprises cyclosporin A, Surfonic CO-42®, and Caraway oil.

Another embodiment comprises cyclosporin A, Surfonic E400-MO®, and Caraway oil.

Another embodiment comprises cyclosporin A, Surfonic L12-3®, and Caraway oil.

Another embodiment comprises cyclosporin A, Surfonic L12-6®, and Caraway oil.

Another embodiment comprises cyclosporin A, Surfonic L12-8®, and Caraway oil.

Another embodiment comprises cyclosporin A, Surfonic L24-12®, and Caraway oil.

Another embodiment comprises cyclosporin A, Surfonic L24-2®, and Caraway oil.

Another embodiment comprises cyclosporin A, Surfonic L24-22®, and Caraway oil.

Another embodiment comprises cyclosporin A, Surfonic L24-3®, and Caraway oil.

Another embodiment comprises cyclosporin A, Surfonic L24-4®, and Caraway oil.

Another embodiment comprises cyclosporin A, Surfonic L24-5®, and Caraway oil.

Another embodiment comprises cyclosporin A, Surfonic L24-7®, and Caraway oil.

Another embodiment comprises cyclosporin A, Surfonic L24-7.1®, and Caraway oil.

Another embodiment comprises cyclosporin A, Surfonic L24-9®, and Caraway oil.

Another embodiment comprises cyclosporin A, Surfonic LF-17®, and Caraway oil.

Another embodiment comprises cyclosporin A, Surfonic LF-18®, and Caraway oil.

Another embodiment comprises cyclosporin A, Surfonic LF-37®, and Caraway oil.

Another embodiment comprises cyclosporin A, Surfonic LF-41®, and Caraway oil.

Another embodiment comprises cyclosporin A, Surfonic LF-42®, and Caraway oil.

Another embodiment comprises cyclosporin A, Surfonic LF-47®, and Caraway oil.

Another embodiment comprises cyclosporin A, Surfonic LF-68®, and Caraway oil.

Another embodiment comprises cyclosporin A, Surfonic N-10®, and Caraway oil.

Another embodiment comprises cyclosporin A, Surfonic N-100®, and Caraway oil.

Another embodiment comprises cyclosporin A, Surfonic N-102®, and Caraway oil.

Another embodiment comprises cyclosporin A, Surfonic N-120®, and Caraway oil.

Another embodiment comprises cyclosporin A, Surtonic N-150®, and Caraway oil.

Another embodiment comprises cyclosporin A, Surfonic N-200®, and Caraway oil.

Another embodiment comprises cyclosporin A, Surfonic N-300®, and Caraway oil.

Another embodiment comprises cyclosporin A, Surfonic N-31.5®, and Caraway oil.

Another embodiment comprises cyclosporin A, Surtonic N-40®, and Caraway oil.

Another embodiment comprises cyclosporin A, Surtonic N-400®, and Caraway oil.

Another embodiment comprises cyclosporin A, Surfonic N-60®, and Caraway oil.

Another embodiment comprises cyclosporin A, Surfonic N-85®, and Caraway oil.

Another embodiment comprises cyclosporin A, Surfonic N-95®, and Caraway oil.

Another embodiment comprises cyclosporin A, Surtonic NB-18®, and Caraway oil.

Another embodiment comprises cyclosporin A, Surfonic NB-23®, and Caraway oil.

Another embodiment comprises cyclosporin A, Surfonic NB-25®, and Caraway oil.

Another embodiment comprises cyclosporin A, Surfonic NB-307®, and Caraway oil.

Another embodiment comprises cyclosporin A, Surfonic NB-31®, and Caraway oil.

Another embodiment comprises cyclosporin A, Surfonic NB-41®, and Caraway oil.

Another embodiment comprises cyclosporin A, Surfonic NB-43®, and Caraway oil.

Another embodiment comprises cyclosporin A, Surfonic OP-100®, and Caraway oil.

Another embodiment comprises cyclosporin A, Surfonic OP-120®, and Caraway oil.

Another embodiment comprises cyclosporin A, Surfonic OP-15®, and Caraway oil.

Another embodiment comprises cyclosporin A, Surfonic OP-35®, and Caraway oil.

Another embodiment comprises cyclosporin A, Surfonic OP-70®, and Caraway oil.

Another embodiment comprises cyclosporin A, Surfonic P1®, and Caraway oil.

Another embodiment comprises cyclosporin A, Surfonic P3®, and Caraway oil.

Another embodiment comprises cyclosporin A, Surfonic P5®, and Caraway oil.

Another embodiment comprises cyclosporin A, Surfonic P6®, and Caraway oil.

Another embodiment comprises cyclosporin A, Surfonic POA-17R2®, and Caraway oil.

Another embodiment comprises cyclosporin A, Surfonic POA-25R2®, and Caraway oil.

Another embodiment comprises cyclosporin A, Surfonic TDA-11®, and Caraway oil.

Another embodiment comprises cyclosporin A, Surfonic TDA-3B®, and Caraway oil.

Another embodiment comprises cyclosporin A, Surfonic TDA-6®, and Caraway oil.

Another embodiment comprises cyclosporin A, Surfonic TDA-6/88®, and Caraway oil.

Another embodiment comprises cyclosporin A, Surfonic TDA-8®, and Caraway oil.

Another embodiment comprises cyclosporin A, Surfonic TDA-8/90®, and Caraway oil.

Another embodiment comprises cyclosporin A, Surfonic TDA-9®, and Caraway oil.

Another embodiment comprises cyclosporin A, Surfonic TX-CCN®, and Caraway oil.

Another embodiment comprises cyclosporin A, Tagat CH 40®, and Caraway oil.

Another embodiment comprises cyclosporin A, Tagat CH 60®, and Caraway oil.

Another embodiment comprises cyclosporin A, Tagat L 2®, and Caraway oil.

Another embodiment comprises cyclosporin A, Tagat S®, and Caraway oil.

Another embodiment comprises cyclosporin A, Tagat S 2®, and Caraway oil.

Another embodiment comprises cyclosporin A, Tally 100 Plus®, and Caraway oil.

Another embodiment comprises cyclosporin A, T-Det A106®, and Caraway oil.

Another embodiment comprises cyclosporin A, T-Det Al 109®, and Caraway oil.

Another embodiment comprises cyclosporin A, T-Det A136®, and Caraway oil.

Another embodiment comprises cyclosporin A, T-Det A139®, and Caraway oil.

Another embodiment comprises cyclosporin A, T-Det A2412®, and Caraway oil.

Another embodiment comprises cyclosporin A, T-Det A243®, and Caraway oil.

Another embodiment comprises cyclosporin A, T-Det A247®, and Caraway oil.

Another embodiment comprises cyclosporin A, T-Det A249®, and Caraway oil.

Another embodiment comprises cyclosporin A, T-Det A467®, and Caraway oil.

Another embodiment comprises cyclosporin A, T-Det COE®, and Caraway oil.

Another embodiment comprises cyclosporin A, T-Det DD-10®, and Caraway oil.

Another embodiment comprises cyclosporin A, T-Det DD-5®, and Caraway oil.

Another embodiment comprises cyclosporin A, T-Det DD-7®, and Caraway oil.

Another embodiment comprises cyclosporin A, T-Det EPO-104®, and Caraway oil.

Another embodiment comprises cyclosporin A, T-Det EPO-61®, and Caraway oil.

Another embodiment comprises cyclosporin A, T-Det EPO-62®, and Caraway oil.

Another embodiment comprises cyclosporin A, T-Det EPO-64®, and Caraway oil.

Another embodiment comprises cyclosporin A, T-Det N-1.5®, and Caraway oil.

Another embodiment comprises cyclosporin A, T-Det N-10.5®, and Caraway oil.

Another embodiment comprises cyclosporin A, T-Det N-100®, and Caraway oil.

Another embodiment comprises cyclosporin A, T-Det N-1007®, and Caraway oil.

Another embodiment comprises cyclosporin A, T-Det N-12®, and Caraway oil.

Another embodiment comprises cyclosporin A, T-Det N-14®, and Caraway oil.

Another embodiment comprises cyclosporin A, T-Det N-20®, and Caraway oil.

Another embodiment comprises cyclosporin A, T-Det N-30®, and Caraway oil.

Another embodiment comprises cyclosporin A, T-Det N-307®, and Caraway oil.

Another embodiment comprises cyclosporin A, T-Det N-4®, and Caraway oil.

Another embodiment comprises cyclosporin A, T-Det N-40®, and Caraway oil.

Another embodiment comprises cyclosporin A, T-Det N-407®, and Caraway oil.

Another embodiment comprises cyclosporin A, T-Det N-50®, and Caraway oil.

Another embodiment comprises cyclosporin A, T-Det N-507®, and Caraway oil.

Another embodiment comprises cyclosporin A, T-Det N-6®, and Caraway oil.

Another embodiment comprises cyclosporin A, T-Det N-70®, and Caraway oil.

Another embodiment comprises cyclosporin A, T-Det N-8®, and Caraway oil.

Another embodiment comprises cyclosporin A, T-Det N-9.5®, and Caraway oil.

Another embodiment comprises cyclosporin A, T-Det O-12®, and Caraway oil.

Another embodiment comprises cyclosporin A, T-Det O-165®, and Caraway oil.

Another embodiment comprises cyclosporin A, T-Det O-307®, and Caraway oil.

Another embodiment comprises cyclosporin A, T-Det O-40®, and Caraway oil.

Another embodiment comprises cyclosporin A, T-Det O-407®, and Caraway oil.

Another embodiment comprises cyclosporin A, T-Det O-6®, and Caraway oil.

Another embodiment comprises cyclosporin A, T-Det O-8®, and Caraway oil.

Another embodiment comprises cyclosporin A, T-Det O-9®, and Caraway oil.

Another embodiment comprises cyclosporin A, Tegin®, and Caraway oil.

Another embodiment comprises cyclosporin A, Tergitol 15-S-12®, and Caraway oil.

Another embodiment comprises cyclosporin A, Tergitol 15-S-15®, and Caraway oil.

Another embodiment comprises cyclosporin A, Tergitol 15-S-20®, and Caraway oil.

Another embodiment comprises cyclosporin A, Tergitol 15-S-20 (80AQ)®, and Caraway oil.

Another embodiment comprises cyclosporin A, Tergitol 15-S-3®, and Caraway oil.

Another embodiment comprises cyclosporin A, Tergitol 15-S-30®, and Caraway oil.

Another embodiment comprises cyclosporin A, Tergitol 15-S-40®, and Caraway oil.

Another embodiment comprises cyclosporin A, Tergitol 15-S-40 (70AQ)®, and Caraway oil.

Another embodiment comprises cyclosporin A, Tergitol 15-S-5®, and Caraway oil.

Another embodiment comprises cyclosporin A, Tergitol 15-S-7®, and Caraway oil.

Another embodiment comprises cyclosporin A, Tergitol 15-S-9®, and Caraway oil.

Another embodiment comprises cyclosporin A, Tergitol L-101®, and Caraway oil.

Another embodiment comprises cyclosporin A, Tergitol L-61®, and Caraway oil.

Another embodiment comprises cyclosporin A, Tergitol L-62®, and Caraway oil.

Another embodiment comprises cyclosporin A, Tergitol L-64®, and Caraway oil.

Another embodiment comprises cyclosporin A, Tergitol L-81®, and Caraway oil.

Another embodiment comprises cyclosporin A, Tergitol NP-10®, and Caraway oil.

Another embodiment comprises cyclosporin A, Tergitol NP-11®, and Caraway oil.

Another embodiment comprises cyclosporin A, Tergitol NP-12®, and Caraway oil.

Another embodiment comprises cyclosporin A, Tergitol NP-13®, and Caraway oil.

Another embodiment comprises cyclosporin A, Tergitol NP-15®, and Caraway oil.

Another embodiment comprises cyclosporin A, Tergitol NP-30®, and Caraway oil.

Another embodiment comprises cyclosporin A, Tergitol NP-30 (70 AQ)®, and Caraway oil.

Another embodiment comprises cyclosporin A, Tergitol NP-4®, and Caraway oil.

Another embodiment comprises cyclosporin A, Tergitol NP-40®, and Caraway oil.

Another embodiment comprises cyclosporin A, Tergitol NP-40 (70 AQ)®, and Caraway oil.

Another embodiment comprises cyclosporin A, Tergitol NP-50 (70 AQ)®, and Caraway oil.

Another embodiment comprises cyclosporin A, Tergitol NP-55 (70 AQ)®, and Caraway oil.

Another embodiment comprises cyclosporin A, Tergitol NP-6®, and Caraway oil.

Another embodiment comprises cyclosporin A, Tergitol NP-7®, and Caraway oil.

Another embodiment comprises cyclosporin A, Tergitol NP-70 (70 AQ)®, and Caraway oil.

Another embodiment comprises cyclosporin A, Tergitol NP-8®, and Caraway oil.

Another embodiment comprises cyclosporin A, Tergitol NP-9®, and Caraway oil.

Another embodiment comprises cyclosporin A, Tergitol NP-9.5®, and Caraway oil.

Another embodiment comprises cyclosporin A, Tergitol TMN-10®, and Caraway oil.

Another embodiment comprises cyclosporin A, Tergitol TMN-3®, and Caraway oil.

Another embodiment comprises cyclosporin A, Tergitol TMN-6®, and Caraway oil.

Another embodiment comprises cyclosporin A, Tergitol XC®, and Caraway oil.

Another embodiment comprises cyclosporin A, Tergitol XH®, and Caraway oil.

Another embodiment comprises cyclosporin A, Tergitol XJ®, and Caraway oil.

Another embodiment comprises cyclosporin A, Tetronic 1107®, and Caraway oil.

Another embodiment comprises cyclosporin A, Tetronic 1307®, and Caraway oil.

Another embodiment comprises cyclosporin A, Tetronic 150R1®, and Caraway oil.

Another embodiment comprises cyclosporin A, Tetronic 304®, and Caraway oil.

Another embodiment comprises cyclosporin A, Tetronic 701®, and Caraway oil.

Another embodiment comprises cyclosporin A, Tetronic 901®, and Caraway oil.

Another embodiment comprises cyclosporin A, Tetronic 904®, and Caraway oil.

Another embodiment comprises cyclosporin A, Tetronic 908®, and Caraway oil.

Another embodiment comprises cyclosporin A, Tetronic 90R4®, and Caraway oil.

Another embodiment comprises cyclosporin A, Tomadol 1-3®, and Caraway oil.

Another embodiment comprises cyclosporin A, Tomadol 1-5®, and Caraway oil.

Another embodiment comprises cyclosporin A, Tomadol 1-7®, and Caraway oil.

Another embodiment comprises cyclosporin A, Tomadol 23-1®, and Caraway oil.

Another embodiment comprises cyclosporin A, Tomadol 23-3®, and Caraway oil.

Another embodiment comprises cyclosporin A, Tomadol 23-5®, and Caraway oil.

Another embodiment comprises cyclosporin A, Tomadol 23-6.5®, and Caraway oil.

Another embodiment comprises cyclosporin A, Tomadol 25-12®, and Caraway oil.

Another embodiment comprises cyclosporin A, Tomadol 25-3®, and Caraway oil.

Another embodiment comprises cyclosporin A, Tomadol 25-7®, and Caraway oil.

Another embodiment comprises cyclosporin A, Tomadol 25-9®, and Caraway oil.

Another embodiment comprises cyclosporin A, Tomadol 45-13®, and Caraway oil.

Another embodiment comprises cyclosporin A, Tomadol 45-2.5®, and Caraway oil.

Another embodiment comprises cyclosporin A, Tomadol 45-7®, and Caraway oil.

Another embodiment comprises cyclosporin A, Tomadol 91-2.5®, and Caraway oil.

Another embodiment comprises cyclosporin A, Tomadol 91-6®, and Caraway oil.

Another embodiment comprises cyclosporin A, Tomadol 91-8®, and Caraway oil.

Another embodiment comprises cyclosporin A, Triton X-100®, and Caraway oil.

Another embodiment comprises cyclosporin A, Triton X-102®, and Caraway oil.

Another embodiment comprises cyclosporin A, Triton X-114®, and Caraway oil.

Another embodiment comprises cyclosporin A, Triton X-15®, and Caraway oil.

Another embodiment comprises cyclosporin A, Triton X-165 (70%)®, and Caraway oil.

Another embodiment comprises cyclosporin A, Triton X-207®, and Caraway oil.

Another embodiment comprises cyclosporin A, Triton X-305 (70%)®, and Caraway oil.

Another embodiment comprises cyclosporin A, Triton X-35®, and Caraway oil.

Another embodiment comprises cyclosporin A, Triton X-405 (70%)®, and Caraway oil.

Another embodiment comprises cyclosporin A, Triton X-45®, and Caraway oil.

Another embodiment comprises cyclosporin A, Triton X-705 (70%)®, and Caraway oil.

Another embodiment comprises cyclosporin A, Volpo S-10®, and Caraway oil.

Another embodiment comprises cyclosporin A, Volpo S-2®, and Caraway oil.

Another embodiment comprises cyclosporin A, Volpo S-20®, and Caraway oil.

Another embodiment comprises cyclosporin A, Volpo-10®, and Caraway oil.

Another embodiment comprises cyclosporin A, Volpo-20®, and Caraway oil.

Another embodiment comprises cyclosporin A, Volpo-3®, and Caraway oil.

Another embodiment comprises cyclosporin A, Volpo-5®, and Caraway oil.

Another embodiment comprises cyclosporin A, Abex 2515®, and Rosemary oil.

Another embodiment comprises cyclosporin A, Abex 2525/40®, and Rosemary oil.

Another embodiment comprises cyclosporin A, Abex 2535®, and Rosemary oil.

Another embodiment comprises cyclosporin A, Abex 2545®, and Rosemary oil.

Another embodiment comprises cyclosporin A, Acconon E®, and Rosemary oil.

Another embodiment comprises cyclosporin A, Alfonic 1216CO-9®, and Rosemary oil.

Another embodiment comprises cyclosporin A, Alfonic 1412-3®, and Rosemary oil.

Another embodiment comprises cyclosporin A, Alfonic TDA-12®, and Rosemary oil.

Another embodiment comprises cyclosporin A, Alfonic TDA-3®, and Rosemary oil.

Another embodiment comprises cyclosporin A, Alfonic TDA-4®, and Rosemary oil.

Another embodiment comprises cyclosporin A, Alfonic TDA-6®, and Rosemary oil.

Another embodiment comprises cyclosporin A, Alfonic TDA-7®, and Rosemary oil.

Another embodiment comprises cyclosporin A, Alfonic TDA-8®, and Rosemary oil.

Another embodiment comprises cyclosporin A, Alfonic TDA-9®, and Rosemary oil.

Another embodiment comprises cyclosporin A, Alkamuls 400-DO®, and Rosemary oil.

Another embodiment comprises cyclosporin A, Alkamuls 400-MO/E®, and Rosemary oil.

Another embodiment comprises cyclosporin A, Ameroxol OE-10®, and Rosemary oil.

Another embodiment comprises cyclosporin A, Ameroxol OE-2®, and Rosemary oil.

Another embodiment comprises cyclosporin A, Ameroxol OE-20®, and Rosemary oil.

Another embodiment comprises cyclosporin A, Armix 176®, and Rosemary oil.

Another embodiment comprises cyclosporin A, Armix 180-C®, and Rosemary oil.

Another embodiment comprises cyclosporin A, Armix 183®, and Rosemary oil.

Another embodiment comprises cyclosporin A, Brij 35®, and Rosemary oil.

Another embodiment comprises cyclosporin A, Brij 35 Liquid®, and Rosemary oil.

Another embodiment comprises cyclosporin A, Brij 35 SP®, and Rosemary oil.

Another embodiment comprises cyclosporin A, Canasol MJ 2109®, and Rosemary oil.

Another embodiment comprises cyclosporin A, Canasol MJ 52®, and Rosemary oil.

Another embodiment comprises cyclosporin A, Canasol R 3600®, and Rosemary oil.

Another embodiment comprises cyclosporin A, Canasol R 3603®, and Rosemary oil.

Another embodiment comprises cyclosporin A, Canasol R 4000 H®, and Rosemary oil.

Another embodiment comprises cyclosporin A, Cedepal CA-210®, and Rosemary oil.

Another embodiment comprises cyclosporin A, Cedepal CA-520®, and Rosemary oil.

Another embodiment comprises cyclosporin A, Cedepal CA-630®, and Rosemary oil.

Another embodiment comprises cyclosporin A, Cedepal CA-720®, and Rosemary oil.

Another embodiment comprises cyclosporin A, Cedepal CA-890®, and Rosemary oil.

Another embodiment comprises cyclosporin A, Cedepal CA-897®, and Rosemary oil.

Another embodiment comprises cyclosporin A, Cedepal CO-210®, and Rosemary oil.

Another embodiment comprises cyclosporin A, Cedepal CO-430®, and Rosemary oil.

Another embodiment comprises cyclosporin A, Cedepal CO-530®, and Rosemary oil.

Another embodiment comprises cyclosporin A, Cedepal CO-630®, and Rosemary oil.

Another embodiment comprises cyclosporin A, Cedepal CO-710®, and Rosemary oil.

Another embodiment comprises cyclosporin A, Cedepal CO-730®, and Rosemary oil.

Another embodiment comprises cyclosporin A, Cedepal CO-880®, and Rosemary oil.

Another embodiment comprises cyclosporin A, Cedepal CO-887®, and Rosemary oil.

Another embodiment comprises cyclosporin A, Cedepal CO-890®, and Rosemary oil.

Another embodiment comprises cyclosporin A, Cedepal CO-897®, and Rosemary oil.

Another embodiment comprises cyclosporin A, Cedepal CO-970®, and Rosemary oil.

Another embodiment comprises cyclosporin A, Cedepal CO-977®, and Rosemary oil.

Another embodiment comprises cyclosporin A, Cedepal CO-990®, and Rosemary oil.

Another embodiment comprises cyclosporin A, Cedepal CO-997®, and Rosemary oil.

Another embodiment comprises cyclosporin A, Chemal BP 261®, and Rosemary oil.

Another embodiment comprises cyclosporin A, Chemal BP 262®, and Rosemary oil.

Another embodiment comprises cyclosporin A, Chemal BP 3172®, and Rosemary oil.

Another embodiment comprises cyclosporin A, Chemal BP 3174®, and Rosemary oil.

Another embodiment comprises cyclosporin A, Chemal DA-4®, and Rosemary oil.

Another embodiment comprises cyclosporin A, Chemal DA-6®, and Rosemary oil.

Another embodiment comprises cyclosporin A, Chemal DA-9®, and Rosemary oil.

Another embodiment comprises cyclosporin A, Chemal LA-23®, and Rosemary oil.

Another embodiment comprises cyclosporin A, Chemal LA-4®, and Rosemary oil.

Another embodiment comprises cyclosporin A, Chemal LA-9®, and Rosemary oil.

Another embodiment comprises cyclosporin A, Chemal OA-20G®, and Rosemary oil.

Another embodiment comprises cyclosporin A, Chemal OA-5®, and Rosemary oil.

Another embodiment comprises cyclosporin A, Chemal OA-9®, and Rosemary oil.

Another embodiment comprises cyclosporin A, Chemal TDA-12®, and Rosemary oil.

Another embodiment comprises cyclosporin A, Chemal TDA-15®, and Rosemary oil.

Another embodiment comprises cyclosporin A, Chemal TDA-3®, and Rosemary oil.

Another embodiment comprises cyclosporin A, Chemal TDA-6®, and Rosemary oil.

Another embodiment comprises cyclosporin A, Chemal TDA-9®, and Rosemary oil.

Another embodiment comprises cyclosporin A, Chemax CO-16®, and Rosemary oil.

Another embodiment comprises cyclosporin A, Chemax CO-200/50®, and Rosemary oil.

Another embodiment comprises cyclosporin A, Chemax CO-25®, and Rosemary oil.

Another embodiment comprises cyclosporin A, Chemax CO-30®, and Rosemary oil.

Another embodiment comprises cyclosporin A, Chemax CO-36®, and Rosemary oil.

Another embodiment comprises cyclosporin A, Chemax CO-40®, and Rosemary oil.

Another embodiment comprises cyclosporin A, Chemax CO-5®, and Rosemary oil.

Another embodiment comprises cyclosporin A, Chemax CO-80®, and Rosemary oil.

Another embodiment comprises cyclosporin A, Chemax DNP-150/50®, and Rosemary oil.

Another embodiment comprises cyclosporin A, Chemax DNP-18®, and Rosemary oil.

Another embodiment comprises cyclosporin A, Chemax DNP-8®, and Rosemary oil.

Another embodiment comprises cyclosporin A, Chemax E 200 ML®, and Rosemary oil.

Another embodiment comprises cyclosporin A, Chemax E 200 MO®, and Rosemary oil.

Another embodiment comprises cyclosporin A, Chemax E 400 ML®, and Rosemary oil.

Another embodiment comprises cyclosporin A, Chemax E 400 MO®, and Rosemary oil.

Another embodiment comprises cyclosporin A, Chemax E 600 ML®, and Rosemary oil.

Another embodiment comprises cyclosporin A, Chemax E 600 MO®, and Rosemary oil.

Another embodiment comprises cyclosporin A, Chemax E 1000 MO®, and Rosemary oil.

Another embodiment comprises cyclosporin A, Chemax E 1000 MS®, and Rosemary oil.

Another embodiment comprises cyclosporin A, Chemax E200 MS®, and Rosemary oil.

Another embodiment comprises cyclosporin A, Chemax E400 MS®, and Rosemary oil.

Another embodiment comprises cyclosporin A, Chemax E600 MS®, and Rosemary oil.

Another embodiment comprises cyclosporin A, Chemax HCO-16®, and Rosemary oil.

Another embodiment comprises cyclosporin A, Chemax HCO-200/50®, and Rosemary oil.

Another embodiment comprises cyclosporin A, Chemax HCO-25®, and Rosemary oil.

Another embodiment comprises cyclosporin A, Chemax HCO-5®, and Rosemary oil.

Another embodiment comprises cyclosporin A, Chemax TO-16®, and Rosemary oil.

Another embodiment comprises cyclosporin A, Chemax TO-8®, and Rosemary oil.

Another embodiment comprises cyclosporin A, Colalube 3409®, and Rosemary oil.

Another embodiment comprises cyclosporin A, Colalube 3411®, and Rosemary oil.

Another embodiment comprises cyclosporin A, Colalube 3413®, and Rosemary oil.

Another embodiment comprises cyclosporin A, Colalube 3414®, and Rosemary oil.

Another embodiment comprises cyclosporin A, Colalube 3415®, and Rosemary oil.

Another embodiment comprises cyclosporin A, Colalube 3416®, and Rosemary oil.

Another embodiment comprises cyclosporin A, Colalube 3417®, and Rosemary oil.

Another embodiment comprises cyclosporin A, Delonic NPE-100®, and Rosemary oil.

Another embodiment comprises cyclosporin A, Delonic NPE-30®, and Rosemary oil.

Another embodiment comprises cyclosporin A, Delonic NPE-40®, and Rosemary oil.

Another embodiment comprises cyclosporin A, Delonic OPE-10®, and Rosemary oil.

Another embodiment comprises cyclosporin A, Delonic OPE-12®, and Rosemary oil.

Another embodiment comprises cyclosporin A, Delonic OPE-30®, and Rosemary oil.

Another embodiment comprises cyclosporin A, Delonic OPE-40®, and Rosemary oil.

Another embodiment comprises cyclosporin A, Delonic OPE-5®, and Rosemary oil.

Another embodiment comprises cyclosporin A, Delonic OPE-7.5®, and Rosemary oil.

Another embodiment comprises cyclosporin A, DePeg 16-C®, and Rosemary oil.

Another embodiment comprises cyclosporin A, DePeg 25-CO®, and Rosemary oil.

Another embodiment comprises cyclosporin A, DePeg 30-CO®, and Rosemary oil.

Another embodiment comprises cyclosporin A, DePeg 40-CO®, and Rosemary oil.

Another embodiment comprises cyclosporin A, DePeg 5-CO®, and Rosemary oil.

Another embodiment comprises cyclosporin A, DePeg 80-CO®, and Rosemary oil.

Another embodiment comprises cyclosporin A, DeSonic S-100®, and Rosemary oil.

Another embodiment comprises cyclosporin A, DeSonic S-114®, and Rosemary oil.

Another embodiment comprises cyclosporin A, DeSonic S-405®, and Rosemary oil.

Another embodiment comprises cyclosporin A, DeSonic S-45®, and Rosemary oil.

Another embodiment comprises cyclosporin A, DeSonic TDA-9®, and Rosemary oil.

Another embodiment comprises cyclosporin A, DeThox Acid O-14®, and Rosemary oil.

Another embodiment comprises cyclosporin A, DeThox Acid O-16®, and Rosemary oil.

Another embodiment comprises cyclosporin A, DeThox Acid O-9®, and Rosemary oil.

Another embodiment comprises cyclosporin A, DeThox Acid TO-14®, and Rosemary oil.

Another embodiment comprises cyclosporin A, DeThox Acid TO-16.5®, and Rosemary oil.

Another embodiment comprises cyclosporin A, DeThox Acid TO-8.5®, and Rosemary oil.

Another embodiment comprises cyclosporin A, Dur-Em 114®, and Rosemary oil.

Another embodiment comprises cyclosporin A, Dur-Em 117®, and Rosemary oil.

Another embodiment comprises cyclosporin A, Dur-Em 204®, and Rosemary oil.

Another embodiment comprises cyclosporin A, Dur-Em 207®,and Rosemary oil.

Another embodiment comprises cyclosporin A, Dur-Em 207E®, and Rosemary oil.

Another embodiment comprises cyclosporin A, Emerest 2704®, and Rosemary oil.

Another embodiment comprises cyclosporin A, Emerest 2712®, and Rosemary oil.

Another embodiment comprises cyclosporin A, Emerest 2715®, and Rosemary oil.

Another embodiment comprises cyclosporin A, Ethal EH-2®, and Rosemary oil.

Another embodiment comprises cyclosporin A, Ethal EH-5®, and Rosemary oil.

Another embodiment comprises cyclosporin A, Ethal OA-23®, and Rosemary oil.

Another embodiment comprises cyclosporin A, Ethosperse CA-20®, and Rosemary oil.

Another embodiment comprises cyclosporin A, Ethosperse LA-12®, and Rosemary oil.

Another embodiment comprises cyclosporin A, Ethosperse LA-23®, and Rosemary oil.

Another embodiment comprises cyclosporin A, Ethosperse LA-4®, and Rosemary oil.

Another embodiment comprises cyclosporin A, Ethox 3095®, and Rosemary oil.

Another embodiment comprises cyclosporin A, Ethox CO-16®, and Rosemary oil.

Another embodiment comprises cyclosporin A, Ethox CO-200®, and Rosemary oil.

Another embodiment comprises cyclosporin A, Ethox CO-25®, and Rosemary oil.

Another embodiment comprises cyclosporin A, Ethox CO-30®, and Rosemary oil.

Another embodiment comprises cyclosporin A, Ethox CO-36®, and Rosemary oil.

Another embodiment comprises cyclosporin A, Ethox CO-40®, and Rosemary oil.

Another embodiment comprises cyclosporin A, Ethox CO-5®, and Rosemary oil.

Another embodiment comprises cyclosporin A, Ethox DO-14®, and Rosemary oil.

Another embodiment comprises cyclosporin A, Ethox DO-9®, and Rosemary oil.

Another embodiment comprises cyclosporin A, Ethox MA-8®, and Rosemary oil.

Another embodiment comprises cyclosporin A, Ethox Ml-14®, and Rosemary oil.

Another embodiment comprises cyclosporin A, Ethox ML-14®, and Rosemary oil.

Another embodiment comprises cyclosporin A, Ethox ML-5®, and Rosemary oil.

Another embodiment comprises cyclosporin A, Ethox MO-14®, and Rosemary oil.

Another embodiment comprises cyclosporin A, Ethox MO-9®, and Rosemary oil.

Another embodiment comprises cyclosporin A, Ethox MS-14®, and Rosemary oil.

Another embodiment comprises cyclosporin A, Ethox MS-23®, and Rosemary oil.

Another embodiment comprises cyclosporin A, Ethox MS-40®, and Rosemary oil.

Another embodiment comprises cyclosporin A, Ethox MS-8®, and Rosemary oil.

Another embodiment comprises cyclosporin A, Ethox TO-16®, and Rosemary oil.

Another embodiment comprises cyclosporin A, Ethox TO-8®, and Rosemary oil.

Another embodiment comprises cyclosporin A, Ethylan SN-120®, and Rosemary oil.

Another embodiment comprises cyclosporin A, Ethylan SN-70®, and Rosemary oil.

Another embodiment comprises cyclosporin A, Ethylan SN-90®, and Rosemary oil.

Another embodiment comprises cyclosporin A, Flexricin 13®, and Rosemary oil.

Another embodiment comprises cyclosporin A, Flexricin 15®, and Rosemary oil.

Another embodiment comprises cyclosporin A, Genapol 1261®, and Rosemary oil.

Another embodiment comprises cyclosporin A, Generol 122 E25®, and Rosemary oil.

Another embodiment comprises cyclosporin A, Glicepol 160®, and Rosemary oil.

Another embodiment comprises cyclosporin A, Glicepol 560®, and Rosemary oil.

Another embodiment comprises cyclosporin A, Glicepol GMS 20®, and Rosemary oil.

Another embodiment comprises cyclosporin A, Hetoxide BN-13®, and Rosemary oil.

Another embodiment comprises cyclosporin A, Hetoxide DNP-4®, and Rosemary oil.

Another embodiment comprises cyclosporin A, Hetoxide DNP-9.6®, and Rosemary oil.

Another embodiment comprises cyclosporin A, Hetoxide G-26®, and Rosemary oil.

Another embodiment comprises cyclosporin A, Hetoxide G-7®, and Rosemary oil.

Another embodiment comprises cyclosporin A, Hetoxide GT-20®, and Rosemary oil.

Another embodiment comprises cyclosporin A, Hetoxide HC-40®, and Rosemary oil.

Another embodiment comprises cyclosporin A, Hetoxol C-24®, and Rosemary oil.

Another embodiment comprises cyclosporin A, Hetoxol CA-10®, and Rosemary oil.

Another embodiment comprises cyclosporin A, Hetoxol CA-2®, and Rosemary oil.

Another embodiment comprises cyclosporin A, Hetoxol CA-20®, and Rosemary oil.

Another embodiment comprises cyclosporin A, Hetoxol CAWS®, and Rosemary oil.

Another embodiment comprises cyclosporin A, Hetoxol CD-3®, and Rosemary oil.

Another embodiment comprises cyclosporin A, Hetoxol CD-4®, and Rosemary oil.

Another embodiment comprises cyclosporin A, Hetoxol CS-15®, and Rosemary oil.

Another embodiment comprises cyclosporin A, Hetoxol CS-20®, and Rosemary oil.

Another embodiment comprises cyclosporin A, Hetoxol CS-20D®, and Rosemary oil.

Another embodiment comprises cyclosporin A, Hetoxol CS-25®, and Rosemary oil.

Another embodiment comprises cyclosporin A, Hetoxol CS-50®, and Rosemary oil.

Another embodiment comprises cyclosporin A, Hetoxol CS-9®, and Rosemary oil.

Another embodiment comprises cyclosporin A, Hetoxol CSA-15®, and Rosemary oil.

Another embodiment comprises cyclosporin A, Hetoxol D®, and Rosemary oil.

Another embodiment comprises cyclosporin A, Hetoxol G®, and Rosemary oil.

Another embodiment comprises cyclosporin A, Hetoxol J®, and Rosemary oil.

Another embodiment comprises cyclosporin A, Hetoxol L®, and Rosemary oil.

Another embodiment comprises cyclosporin A, Hetoxol L-23®, and Rosemary oil.

Another embodiment comprises cyclosporin A, Hetoxol L-4®, and Rosemary oil.

Another embodiment comprises cyclosporin A, Hetoxol L-9®, and Rosemary oil.

Another embodiment comprises cyclosporin A, Hetoxol LS-9®, and Rosemary oil.

Another embodiment comprises cyclosporin A, Hetoxol M-3®, and Rosemary oil.

Another embodiment comprises cyclosporin A, Hetoxol OA-10 Special®, and Rosemary oil.

Another embodiment comprises cyclosporin A, Hetoxol OA-20 Special®, and Rosemary oil.

Another embodiment comprises cyclosporin A, Hetoxol OA-3 Special®, and Rosemary oil.

Another embodiment comprises cyclosporin A, Hetoxol OA-5 Special®, and Rosemary oil.

Another embodiment comprises cyclosporin A, Hetoxol OL-10®, and Rosemary oil.

Another embodiment comprises cyclosporin A, Hetoxol OL-2®, and Rosemary oil.

Another embodiment comprises cyclosporin A, Hetoxol OL-23®, and Rosemary oil.

Another embodiment comprises cyclosporin A, Hetoxol OL-4®, and Rosemary oil.

Another embodiment comprises cyclosporin A, Hetoxol OL-40®, and Rosemary oil.

Another embodiment comprises cyclosporin A, Hetoxol OL-5®, and Rosemary oil.

Another embodiment comprises cyclosporin A, Hetoxol PLA®, and Rosemary oil.

Another embodiment comprises cyclosporin A, Hetoxol SP-15®, and Rosemary oil.

Another embodiment comprises cyclosporin A, Hetoxol STA-10®, and Rosemary oil.

Another embodiment comprises cyclosporin A, Hetoxol STA-2®, and Rosemary oil.

Another embodiment comprises cyclosporin A, Hetoxol STA-30®, and Rosemary oil.

Another embodiment comprises cyclosporin A, Hetoxol TD-12®, and Rosemary oil.

Another embodiment comprises cyclosporin A, Hetoxol TD-18®, and Rosemary oil.

Another embodiment comprises cyclosporin A, Hetoxol TD-3®, and Rosemary oil.

Another embodiment comprises cyclosporin A, Hetoxol TD-6®, and Rosemary oil.

Another embodiment comprises cyclosporin A, Hetoxol TD-9®, and Rosemary oil.

Another embodiment comprises cyclosporin A, Hetoxol TDEP-15®, and Rosemary oil.

Another embodiment comprises cyclosporin A, Hetoxol TDEP-63®, and Rosemary oil.

Another embodiment comprises cyclosporin A, Iconol 24-12®, and Rosemary oil.

Another embodiment comprises cyclosporin A, Iconol 24-23®, and Rosemary oil.

Another embodiment comprises cyclosporin A, Iconol 24-4®, and Rosemary oil.

Another embodiment comprises cyclosporin A, Iconol DA-4®, and Rosemary oil.

Another embodiment comprises cyclosporin A, Iconol DA-6®, and Rosemary oil.

Another embodiment comprises cyclosporin A, Iconol TDA-10®, and Rosemary oil.

Another embodiment comprises cyclosporin A, Iconol TDA-3®, and Rosemary oil.

Another embodiment comprises cyclosporin A, Iconol TDA-6®, and Rosemary oil.

Another embodiment comprises cyclosporin A, Iconol TDA-8-900%®, and Rosemary oil.

Another embodiment comprises cyclosporin A, Iconol TDA-9®, and Rosemary oil.

Another embodiment comprises cyclosporin A, Igepal CA-210®, and Rosemary oil.

Another embodiment comprises cyclosporin A, Igepal CA-407®, and Rosemary oil.

Another embodiment comprises cyclosporin A, Igepal CA-420®, and Rosemary oil.

Another embodiment comprises cyclosporin A, Igepal CA-520®, and Rosemary oil.

Another embodiment comprises cyclosporin A, Igepal CA-620®, and Rosemary oil.

Another embodiment comprises cyclosporin A, Igepal CA-630®, and Rosemary oil.

Another embodiment comprises cyclosporin A, Igepal CA-720®, and Rosemary oil.

Another embodiment comprises cyclosporin A, Igepal CA-877®, and Rosemary oil.

Another embodiment comprises cyclosporin A, Igepal CA-887®, and Rosemary oil.

Another embodiment comprises cyclosporin A, Igepal CA-897®, and Rosemary oil.

Another embodiment comprises cyclosporin A, Igepal CO-210®, and Rosemary oil.

Another embodiment comprises cyclosporin A, Igepal CO-430®, and Rosemary oil.

Another embodiment comprises cyclosporin A, Igepal CO-520®, and Rosemary oil.

Another embodiment comprises cyclosporin A, Igepal CO-530®, and Rosemary oil.

Another embodiment comprises cyclosporin A, Igepal CO-610®, and Rosemary oil.

Another embodiment comprises cyclosporin A, Igepal CO-630®, and Rosemary oil.

Another embodiment comprises cyclosporin A, Igepal CO-630 Special®, and Rosemary oil.

Another embodiment comprises cyclosporin A, Igepal CO-660®, and Rosemary oil.

Another embodiment comprises cyclosporin A, Igepal CO-710®, and Rosemary oil.

Another embodiment comprises cyclosporin A, Igepal CO-720®, and Rosemary oil.

Another embodiment comprises cyclosporin A, Igepal CO-730®, and Rosemary oil.

Another embodiment comprises cyclosporin A, Igepal CO-738®, and Rosemary oil.

Another embodiment comprises cyclosporin A, Igepal CO-850®, and Rosemary oil.

Another embodiment comprises cyclosporin A, Igepal CO-880®, and Rosemary oil.

Another embodiment comprises cyclosporin A, Igepal CO-887®, and Rosemary oil.

Another embodiment comprises cyclosporin A, Igepal CO-890®, and Rosemary oil.

Another embodiment comprises cyclosporin A, Igepal CO-897®, and Rosemary oil.

Another embodiment comprises cyclosporin A, Igepal CO-970®, and Rosemary oil.

Another embodiment comprises cyclosporin A, Igepal CO-977®, and Rosemary oil.

Another embodiment comprises cyclosporin A, Igepal CO-987®, and Rosemary oil.

Another embodiment comprises cyclosporin A, Igepal CO-990 FLK®, and Rosemary oil.

Another embodiment comprises cyclosporin A, Igepal CO-997®, and Rosemary oil.

Another embodiment comprises cyclosporin A, Igepal DM-430®, and Rosemary oil.

Another embodiment comprises cyclosporin A, Igepal DM-530®, and Rosemary oil.

Another embodiment comprises cyclosporin A, Igepal DM-710®, and Rosemary oil.

Another embodiment comprises cyclosporin A, Igepal DM-730®, and Rosemary oil.

Another embodiment comprises cyclosporin A, Igepal DM-880®, and Rosemary oil.

Another embodiment comprises cyclosporin A, Igepal DM-970 FLK®, and Rosemary oil.

Another embodiment comprises cyclosporin A, Igepal OD-410®, and Rosemary oil.

Another embodiment comprises cyclosporin A, Igepal RC-520®, and Rosemary oil.

Another embodiment comprises cyclosporin A, Igepal RC-620®, and Rosemary oil.

Another embodiment comprises cyclosporin A, Igepal RC-630®, and Rosemary oil.

Another embodiment comprises cyclosporin A, Lexemul CS-20®, and Rosemary oil.

Another embodiment comprises cyclosporin A, Lipo GMS 450®, and Rosemary oil.

Another embodiment comprises cyclosporin A, Lipo Polyglycol 1000®, and Rosemary oil.

Another embodiment comprises cyclosporin A, Lipo Polyglycol 200®, and Rosemary oil.

Another embodiment comprises cyclosporin A, Lipo Polyglycol 300®, and Rosemary oil.

Another embodiment comprises cyclosporin A, Lipo Polyglycol 3350®, and Rosemary oil.

Another embodiment comprises cyclosporin A, Lipo Polyglycol 400®, and Rosemary oil.

Another embodiment comprises cyclosporin A, Lipo Polyglycol 600®, and Rosemary oil.

Another embodiment comprises cyclosporin A, Lipocol C-10®, and Rosemary oil.

Another embodiment comprises cyclosporin A, Lipocol C-2®, and Rosemary oil.

Another embodiment comprises cyclosporin A, Lipocol C-20®, and Rosemary oil.

Another embodiment comprises cyclosporin A, Lipocol L-12®, and Rosemary oil.

Another embodiment comprises cyclosporin A, Lipocol L-23®, and Rosemary oil.

Another embodiment comprises cyclosporin A, Lipocol L-4®, and Rosemary oil.

Another embodiment comprises cyclosporin A, Lipocol O-10®, and Rosemary oil.

Another embodiment comprises cyclosporin A, Lipocol O-2®, and Rosemary oil.

Another embodiment comprises cyclosporin A, Lipocol O-20®, and Rosemary oil.

Another embodiment comprises cyclosporin A, Lipocol O-5®, and Rosemary oil.

Another embodiment comprises cyclosporin A, Lipocol S-10®, and Rosemary oil.

Another embodiment comprises cyclosporin A, Lipocol S-2®, and Rosemary oil.

Another embodiment comprises cyclosporin A, Lipocol S-20®, and Rosemary oil.

Another embodiment comprises cyclosporin A, Lipocol SC-15®, and Rosemary oil.

Another embodiment comprises cyclosporin A, Lipocol SC-20®, and Rosemary oil.

Another embodiment comprises cyclosporin A, Lipocol SC-4®, and Rosemary oil.

Another embodiment comprises cyclosporin A, Lipocol TD-12®, and Rosemary oil.

Another embodiment comprises cyclosporin A, Lumulse 40-O K®, and Rosemary oil.

Another embodiment comprises cyclosporin A, Lumulse 42-OK®, and Rosemary oil.

Another embodiment comprises cyclosporin A, Lumulse GMT-40®, and Rosemary oil.

Another embodiment comprises cyclosporin A, Lumulse PEG 1450®, and Rosemary oil.

Another embodiment comprises cyclosporin A, Lumulse PEG 1450 NF®, and Rosemary oil.

Another embodiment comprises cyclosporin A, Lumulse PEG 8000®, and Rosemary oil.

Another embodiment comprises cyclosporin A, Lumulse PGO®, and Rosemary oil.

Another embodiment comprises cyclosporin A, Lumulse POE (2) Oleyl Amine®, and Rosemary oil.

Another embodiment comprises cyclosporin A, Magrabar PGO-104®, and Rosemary oil.

Another embodiment comprises cyclosporin A, Mapeg 200 ML®, and Rosemary oil.

Another embodiment comprises cyclosporin A, Mapeg 400 MO®, and Rosemary oil.

Another embodiment comprises cyclosporin A, Mapeg 400 MOT®, and Rosemary oil.

Another embodiment comprises cyclosporin A, Mapeg 400DO®, and Rosemary oil.

Another embodiment comprises cyclosporin A, Mapeg 400DOT®, and Rosemary oil.

Another embodiment comprises cyclosporin A, Mapeg 600DOT®, and Rosemary oil.

Another embodiment comprises cyclosporin A, Marlox B 24/80®, and Rosemary oil.

Another embodiment comprises cyclosporin A, Marlox FK 86®, and Rosemary oil.

Another embodiment comprises cyclosporin A, Marlox MO 154®, and Rosemary oil.

Another embodiment comprises cyclosporin A, Mazox CDA CG®, and Rosemary oil.

Another embodiment comprises cyclosporin A, Monoglycerides Glyceryl Monestearate®, and Rosemary oil.

Another embodiment comprises cyclosporin A, Myrj 45®, and Rosemary oil.

Another embodiment comprises cyclosporin A, Myrj 52®, and Rosemary oil.

Another embodiment comprises cyclosporin A, Myrj 52S®, and Rosemary oil.

Another embodiment comprises cyclosporin A, Myrj 53®, and Rosemary oil.

Another embodiment comprises cyclosporin A, Myrj 59®, and Rosemary oil.

Another embodiment comprises cyclosporin A, Norfox NP-12®, and Rosemary oil.

Another embodiment comprises cyclosporin A, Nortox NP-4®, and Rosemary oil.

Another embodiment comprises cyclosporin A, Norfox NP-6®, and Rosemary oil.

Another embodiment comprises cyclosporin A, Norfox NP-887®, and Rosemary oil.

Another embodiment comprises cyclosporin A, Norfox NP-9®, and Rosemary oil.

Another embodiment comprises cyclosporin A, Norfox NP-977®, and Rosemary oil.

Another embodiment comprises cyclosporin A, Norfox OP-100®, and Rosemary oil.

Another embodiment comprises cyclosporin A, Norfox OP-102®, and Rosemary oil.

Another embodiment comprises cyclosporin A, Norfox OP-114®, and Rosemary oil.

Another embodiment comprises cyclosporin A, Norox OP-45®, and Rosemary oil.

Another embodiment comprises cyclosporin A, Pegosperse 100-L®, and Rosemary oil.

Another embodiment comprises cyclosporin A, Pegosperse 100-O®, and Rosemary oil.

Another embodiment comprises cyclosporin A, Pegosperse 100-S®, and Rosemary oil.

Another embodiment comprises cyclosporin A, Pegosperse 1500-MS®, and Rosemary oil.

Another embodiment comprises cyclosporin A, Pegosperse 1750-MS®, and Rosemary oil.

Another embodiment comprises cyclosporin A, Pegosperse 200 DL®, and Rosemary oil.

Another embodiment comprises cyclosporin A, Pegosperse 200-ML®, and Rosemary oil.

Another embodiment comprises cyclosporin A, Pegosperse 400-DL®, and Rosemary oil.

Another embodiment comprises cyclosporin A, Pegosperse 400-DO®, and Rosemary oil.

Another embodiment comprises cyclosporin A, Pegosperse 400-DS®, and Rosemary oil.

Another embodiment comprises cyclosporin A, Pegosperse 400-ML®, and Rosemary oil.

Another embodiment comprises cyclosporin A, Pegosperse 400-MO®, and Rosemary oil.

Another embodiment comprises cyclosporin A, Pegosperse 400-MOT®, and Rosemary oil.

Another embodiment comprises cyclosporin A, Pegosperse 400-MS®, and Rosemary oil.

Another embodiment comprises cyclosporin A, Pegosperse 50-DS®, and Rosemary oil.

Another embodiment comprises cyclosporin A, Pegosperse 50-MS®, and Rosemary oil.

Another embodiment comprises cyclosporin A, Pegosperse 600-ML®, and Rosemary oil.

Another embodiment comprises cyclosporin A, Pegosperse 600-MS®, and Rosemary oil.

Another embodiment comprises cyclosporin A, Pluratac A-38®, and Rosemary oil.

Another embodiment comprises cyclosporin A, Plurafac B-25-5®, and Rosemary oil.

Another embodiment comprises cyclosporin A, Plurafac B-26®, and Rosemary oil.

Another embodiment comprises cyclosporin A, Plurafac D-25®, and Rosemary oil.

Another embodiment comprises cyclosporin A, Plurafac LF 131®, and Rosemary oil.

Another embodiment comprises cyclosporin A, Plurafac LF 4030®, and Rosemary oil.

Another embodiment comprises cyclosporin A, Plurafac LF 7000®, and Rosemary oil.

Another embodiment comprises cyclosporin A, Plurafac RA-20®, and Rosemary oil.

Another embodiment comprises cyclosporin A, Plurafac RA-30®, and Rosemary oil.

Another embodiment comprises cyclosporin A, Plurafac RA-40®, and Rosemary oil.

Another embodiment comprises cyclosporin A, Pluronic 10R5®, and Rosemary oil.

Another embodiment comprises cyclosporin A, Pluronic 17R2s, and Rosemary oil.

Another embodiment comprises cyclosporin A, Pluronic 17R4®, and Rosemary oil.

Another embodiment comprises cyclosporin A, Pluronic 25R2®, and Rosemary oil.

Another embodiment comprises cyclosporin A, Pluronic 25R4®, and Rosemary oil.

Another embodiment comprises cyclosporin A, Pluronic 31R1®, and Rosemary oil.

Another embodiment comprises cyclosporin A, Pluronic F108®, and Rosemary oil.

Another embodiment comprises cyclosporin A, Pluronic F127®, and Rosemary oil.

Another embodiment comprises cyclosporin A, Pluronic F38®, and Rosemary oil.

Another embodiment comprises cyclosporin A, Pluronic F68®, and Rosemary oil.

Another embodiment comprises cyclosporin A, Pluronic F68LF®, and Rosemary oil.

Another embodiment comprises cyclosporin A, Pluronic F77®, and Rosemary oil.

Another embodiment comprises cyclosporin A, Pluronic F87®, and Rosemary oil.

Another embodiment comprises cyclosporin A, Pluronic F88®, and Rosemary oil.

Another embodiment comprises cyclosporin A, Pluronic F98®, and Rosemary oil.

Another embodiment comprises cyclosporin A, Pluronic L10®, and Rosemary oil.

Another embodiment comprises cyclosporin A, Pluronic L101®, and Rosemary oil.

Another embodiment comprises cyclosporin A, Pluronic L121®, and Rosemary oil.

Another embodiment comprises cyclosporin A, Pluronic L31®, and Rosemary oil.

Another embodiment comprises cyclosporin A, Pluronic L35®, and Rosemary oil.

Another embodiment comprises cyclosporin A, Pluronic L43®, and Rosemary oil.

Another embodiment comprises cyclosporin A, Pluronic L44®, and Rosemary oil.

Another embodiment comprises cyclosporin A, Pluronic L61®, and Rosemary oil.

Another embodiment comprises cyclosporin A, Pluronic L62®, and Rosemary oil.

Another embodiment comprises cyclosporin A, Pluronic L62D®, and Rosemary oil.

Another embodiment comprises cyclosporin A, Pluronic L62LF®, and Rosemary oil.

Another embodiment comprises cyclosporin A, Pluronic L64®, and Rosemary oil.

Another embodiment comprises cyclosporin A, Pluronic L81®, and Rosemary oil.

Another embodiment comprises cyclosporin A, Pluronic L92®, and Rosemary oil.

Another embodiment comprises cyclosporin A, Pluronic P103®, and Rosemary oil.

Another embodiment comprises cyclosporin A, Pluronic P104®, and Rosemary oil.

Another embodiment comprises cyclosporin A, Pluronic P105®, and Rosemary oil.

Another embodiment comprises cyclosporin A, Pluronic P123®, and Rosemary oil.

Another embodiment comprises cyclosporin A, Pluronic P65®, and Rosemary oil.

Another embodiment comprises cyclosporin A, Pluronic P84®, and Rosemary oil.

Another embodiment comprises cyclosporin A, Pluronic P85®, and Rosemary oil.

Another embodiment comprises cyclosporin A, Polyaldo 10-2-P®, and Rosemary oil.

Another embodiment comprises cyclosporin A, Polyaldo DGDO®, and Rosemary oil.

Another embodiment comprises cyclosporin A, Polyaldo HGDS®, and Rosemary oil.

Another embodiment comprises cyclosporin A, Polyaldo TGMS®, and Rosemary oil.

Another embodiment comprises cyclosporin A, Protegin V®, and Rosemary oil.

Another embodiment comprises cyclosporin A, Protegin W®, and Rosemary oil.

Another embodiment comprises cyclosporin A, Protegin WX®, and Rosemary oil.

Another embodiment comprises cyclosporin A, Protegin XV®, and Rosemary oil.

Another embodiment comprises cyclosporin A, Rhodasurf BC-420®, and Rosemary oil.

Another embodiment comprises cyclosporin A, Rhodasur BC-610®, and Rosemary oil.

Another embodiment comprises cyclosporin A, Rhodasurf BC-630®, and Rosemary oil.

Another embodiment comprises cyclosporin A, Rhodasurf BC-720®, and Rosemary oil.

Another embodiment comprises cyclosporin A, Rhodasurf BC-840®, and Rosemary oil.

Another embodiment comprises cyclosporin A, Rhodasurf L 1®, and Rosemary oil.

Another embodiment comprises cyclosporin A, Rhodasurf L 12®, and Rosemary oil.

Another embodiment comprises cyclosporin A, Rhodasurf L 2®, and Rosemary oil.

Another embodiment comprises cyclosporin A, Rhodasurf L 20®, and Rosemary oil.

Another embodiment comprises cyclosporin A, Rhodasurf L 3®, and Rosemary oil.

Another embodiment comprises cyclosporin A, Rhodasurf L 30®, and Rosemary oil.

Another embodiment comprises cyclosporin A, Rhodasurf L 4®, and Rosemary oil.

Another embodiment comprises cyclosporin A, Rhodasurf L 50®, and Rosemary oil.

Another embodiment comprises cyclosporin A, Rhodasurf L 7/90®, and Rosemary oil.

Another embodiment comprises cyclosporin A, Rhodasurf LA-3®, and Rosemary oil.

Another embodiment comprises cyclosporin A, Rhodasurf LA-7®,and Rosemary oil.

Another embodiment comprises cyclosporin A, Rhodasurf LAN-23®, and Rosemary oil.

Another embodiment comprises cyclosporin A, Rhodasurf ON-870®, and Rosemary oil.

Another embodiment comprises cyclosporin A, Rhodasurf ON-877®, and Rosemary oil.

Another embodiment comprises cyclosporin A, Rhodasurf TDA-8/5®, and Rosemary oil.

Another embodiment comprises cyclosporin A, Ritapro 100®, and Rosemary oil.

Another embodiment comprises cyclosporin A, Ritapro 165®, and Rosemary oil.

Another embodiment comprises cyclosporin A, Ritapro 200®, and Rosemary oil.

Another embodiment comprises cyclosporin A, Ritapro 300®, and Rosemary oil.

Another embodiment comprises cyclosporin A, Ritoleth 10®, and Rosemary oil.

Another embodiment comprises cyclosporin A, Ritoleth 2®, and Rosemary oil.

Another embodiment comprises cyclosporin A, Ritoleth 20®, and Rosemary oil.

Another embodiment comprises cyclosporin A, Ritoleth 5®, and Rosemary oil.

Another embodiment comprises cyclosporin A, Santone 10-10-0®, and Rosemary oil.

Another embodiment comprises cyclosporin A, Santone 3-1-S XTR®, and Rosemary oil.

Another embodiment comprises cyclosporin A, Santone 3-1-SH®, and Rosemary oil.

Another embodiment comprises cyclosporin A, Santone 8-1-O®, and Rosemary oil.

Another embodiment comprises cyclosporin A, Solulan 16®, and Rosemary oil.

Another embodiment comprises cyclosporin A, Solulan 75®, and Rosemary oil.

Another embodiment comprises cyclosporin A, Solulan C-24®, and Rosemary oil.

Another embodiment comprises cyclosporin A, Solulan L-575®, and Rosemary oil.

Another embodiment comprises cyclosporin A, Standamox CAW®, and Rosemary oil.

Another embodiment comprises cyclosporin A, Surfine AZI-A®, and Rosemary oil.

Another embodiment comprises cyclosporin A, Surfonic CO-15®, and Rosemary oil.

Another embodiment comprises cyclosporin A, Surfonic CO-25®, and Rosemary oil.

Another embodiment comprises cyclosporin A, Surfonic CO-30®, and Rosemary oil.

Another embodiment comprises cyclosporin A, Surfonic CO-36®, and Rosemary oil.

Another embodiment comprises cyclosporin A, Surfonic CO-42®, and Rosemary oil.

Another embodiment comprises cyclosporin A, Surfonic E400-MO®, and Rosemary oil.

Another embodiment comprises cyclosporin A, Surfonic L12-3®, and Rosemary oil.

Another embodiment comprises cyclosporin A, Surfonic L12-6®, and Rosemary oil.

Another embodiment comprises cyclosporin A, Surfonic L12-8®, and Rosemary oil.

Another embodiment comprises cyclosporin A, Surfonic L24-12®, and Rosemary oil.

Another embodiment comprises cyclosporin A, Surfonic L24-2®, and Rosemary oil.

Another embodiment comprises cyclosporin A, Surfonic L24-22®, and Rosemary oil.

Another embodiment comprises cyclosporin A, Surfonic L24-3®, and Rosemary oil.

Another embodiment comprises cyclosporin A, Surfonic L24-4®, and Rosemary oil.

Another embodiment comprises cyclosporin A, Surfonic L24-5®, and Rosemary oil.

Another embodiment comprises cyclosporin A, Surfonic L24-7®, and Rosemary oil.

Another embodiment comprises cyclosporin A, Surfonic L24-7.1®, and Rosemary oil.

Another embodiment comprises cyclosporin A, Surfonic L24-9®, and Rosemary oil.

Another embodiment comprises cyclosporin A, Surfonic LF-17®, and Rosemary oil.

Another embodiment comprises cyclosporin A, Surfonic LF-18®, and Rosemary oil.

Another embodiment comprises cyclosporin A, Surfonic LF-37®, and Rosemary oil.

Another embodiment comprises cyclosporin A, Surfonic LF-41®, and Rosemary oil.

Another embodiment comprises cyclosporin A, Surfonic LF-42®, and Rosemary oil.

Another embodiment comprises cyclosporin A, Surfonic LF-47®, and Rosemary oil.

Another embodiment comprises cyclosporin A, Surfonic LF-68®, and Rosemary oil.

Another embodiment comprises cyclosporin A, Surfonic N-10®, and Rosemary oil.

Another embodiment comprises cyclosporin A, Surfonic N-100®, and Rosemary oil.

Another embodiment comprises cyclosporin A, Surfonic N-102®,and Rosemary oil.

Another embodiment comprises cyclosporin A, Surfonic N-120®, and Rosemary oil.

Another embodiment comprises cyclosporin A, Surfonic N-150®, and Rosemary oil.

Another embodiment comprises cyclosporin A, Surfonic N-200®, and Rosemary oil.

Another embodiment comprises cyclosporin A, Surfonic N-300®, and Rosemary oil.

Another embodiment comprises cyclosporin A, Surfonic N-31.5®, and Rosemary oil.

Another embodiment comprises cyclosporin A, Surfonic N-40®, and Rosemary oil.

Another embodiment comprises cyclosporin A, Surfonic N-400®, and Rosemary oil.

Another embodiment comprises cyclosporin A, Surfonic N-60®, and Rosemary oil.

Another embodiment comprises cyclosporin A, Surfonic N-85®, and Rosemary oil.

Another embodiment comprises cyclosporin A, Surfonic N-95®, and Rosemary oil.

Another embodiment comprises cyclosporin A, Surfonic NB-18®, and Rosemary oil.

Another embodiment comprises cyclosporin A, Surfonic NB-23®, and Rosemary oil.

Another embodiment comprises cyclosporin A, Surfonic NB-25®, and Rosemary oil.

Another embodiment comprises cyclosporin A, Surfonic NB-307®, and Rosemary oil.

Another embodiment comprises cyclosporin A, Surfonic NB-31®, and Rosemary oil.

Another embodiment comprises cyclosporin A, Surfonic NB-41®, and Rosemary oil.

Another embodiment comprises cyclosporin A, Surfonic NB-43®, and Rosemary oil.

Another embodiment comprises cyclosporin A, Surfonic OP-100®, and Rosemary oil.

Another embodiment comprises cyclosporin A, Surfonic OP-120®, and Rosemary oil.

Another embodiment comprises cyclosporin A, Surfonic OP-15®, and Rosemary oil.

Another embodiment comprises cyclosporin A, Surfonic OP-35®, and Rosemary oil.

Another embodiment comprises cyclosporin A, Surfonic OP-70®, and Rosemary oil.

Another embodiment comprises cyclosporin A, Surfonic P1®, and Rosemary oil.

Another embodiment comprises cyclosporin A, Surfonic P3®, and Rosemary oil.

Another embodiment comprises cyclosporin A, Surfonic P5®, and Rosemary oil.

Another embodiment comprises cyclosporin A, Surfonic P6®, and Rosemary oil.

Another embodiment comprises cyclosporin A, Surfonic POA-17R2®, and Rosemary oil.

Another embodiment comprises cyclosporin A, Surfonic POA-25R2®, and Rosemary oil.

Another embodiment comprises cyclosporin A, Surfonic TDA-11®, and Rosemary oil.

Another embodiment comprises cyclosporin A, Surfonic TDA-3B®, and Rosemary oil.

Another embodiment comprises cyclosporin A, Surfonic TDA-6®, and Rosemary oil.

Another embodiment comprises cyclosporin A, Surfonic TDA-6/88®, and Rosemary oil.

Another embodiment comprises cyclosporin A, Surfonic TDA-8®, and Rosemary oil.

Another embodiment comprises cyclosporin A, Surfonic TDA-8/90®, and Rosemary oil.

Another embodiment comprises cyclosporin A, Surfonic TDA-9®, and Rosemary oil.

Another embodiment comprises cyclosporin A, Surfonic TX-CCN®, and Rosemary oil.

Another embodiment comprises cyclosporin A, Tagat CH 40®, and Rosemary oil.

Another embodiment comprises cyclosporin A, Tagat CH 60®, and Rosemary oil.

Another embodiment comprises cyclosporin A, Tagat L 2®, and Rosemary oil.

Another embodiment comprises cyclosporin A, Tagat S®, and Rosemary oil.

Another embodiment comprises cyclosporin A, Tagat S 2®, and Rosemary oil.

Another embodiment comprises cyclosporin A, Tally 100 Plus®, and Rosemary oil.

Another embodiment comprises cyclosporin A, T-Det A106®, and Rosemary oil.

Another embodiment comprises cyclosporin A, T-Det A109®, and Rosemary oil.

Another embodiment comprises cyclosporin A, T-Det A136®, and Rosemary oil.

Another embodiment comprises cyclosporin A, T-Det A139®, and Rosemary oil.

Another embodiment comprises cyclosporin A, T-Det A2412®, and Rosemary oil.

Another embodiment comprises cyclosporin A, T-Det A243®, and Rosemary oil.

Another embodiment comprises cyclosporin A, T-Det A247®, and Rosemary oil.

Another embodiment comprises cyclosporin A, T-Det A249®, and Rosemary oil.

Another embodiment comprises cyclosporin A, T-Det A467®, and Rosemary oil.

Another embodiment comprises cyclosporin A, T-Det COE®, and Rosemary oil.

Another embodiment comprises cyclosporin A, T-Det DD-10®, and Rosemary oil.

Another embodiment comprises cyclosporin A, T-Det DD-5®, and Rosemary oil.

Another embodiment comprises cyclosporin A, T-Det DD-7®, and Rosemary oil.

Another embodiment comprises cyclosporin A, T-Det EPO-104®, and Rosemary oil.

Another embodiment comprises cyclosporin A, T-Det EPO-61®, and Rosemary oil.

Another embodiment comprises cyclosporin A, T-Det EPO-62®, and Rosemary oil.

Another embodiment comprises cyclosporin A, T-Det EPO-64®, and Rosemary oil.

Another embodiment comprises cyclosporin A, T-Det N-1.5®, and Rosemary oil.

Another embodiment comprises cyclosporin A, T-Det N-10.5®, and Rosemary oil.

Another embodiment comprises cyclosporin A, T-Det N-100®, and Rosemary oil.

Another embodiment comprises cyclosporin A, T-Det N-1007®, and Rosemary oil.

Another embodiment comprises cyclosporin A, T-Det N-12®, and Rosemary oil.

Another embodiment comprises cyclosporin A, T-Det N-14®, and Rosemary oil.

Another embodiment comprises cyclosporin A, T-Det N-20®, and Rosemary oil.

Another embodiment comprises cyclosporin A, T-Det N-30®, and Rosemary oil.

Another embodiment comprises cyclosporin A, T-Det N-307®, and Rosemary oil.

Another embodiment comprises cyclosporin A, T-Det N-4®, and Rosemary oil.

Another embodiment comprises cyclosporin A, T-Det N-40®, and Rosemary oil.

Another embodiment comprises cyclosporin A, T-Det N-407®, and Rosemary oil.

Another embodiment comprises cyclosporin A, T-Det N-50®, and Rosemary oil.

Another embodiment comprises cyclosporin A, T-Det N-507®, and Rosemary oil.

Another embodiment comprises cyclosporin A, T-Det N-6®, and Rosemary oil.

Another embodiment comprises cyclosporin A, T-Det N-70®, and Rosemary oil.

Another embodiment comprises cyclosporin A, T-Det N-8®, and Rosemary oil.

Another embodiment comprises cyclosporin A, T-Det N-9.5®, and Rosemary oil.

Another embodiment comprises cyclosporin A, T-Det O-12®, and Rosemary oil.

Another embodiment comprises cyclosporin A, T-Det O-165®, and Rosemary oil.

Another embodiment comprises cyclosporin A, T-Det O-307®, and Rosemary oil.

Another embodiment comprises cyclosporin A, T-Det O-40®, and Rosemary oil.

Another embodiment comprises cyclosporin A, T-Det O-407®, and Rosemary oil.

Another embodiment comprises cyclosporin A, T-Det O-6®, and Rosemary oil.

Another embodiment comprises cyclosporin A, T-Det O-8®, and Rosemary oil.

Another embodiment comprises cyclosporin A, T-Det O-9®, and Rosemary oil.

Another embodiment comprises cyclosporin A, Tegin®, and Rosemary oil.

Another embodiment comprises cyclosporin A, Tergitol 15-S-12®, and Rosemary oil.

Another embodiment comprises cyclosporin A, Tergitol 15-S-15®, and Rosemary oil.

Another embodiment comprises cyclosporin A, Tergitol 15-S-20®, and Rosemary oil.

Another embodiment comprises cyclosporin A, Tergitol 15-S-20 (80AQ)®, and Rosemary oil.

Another embodiment comprises cyclosporin A, Tergitol 15-S-3®, and Rosemary oil.

Another embodiment comprises cyclosporin A, Tergitol 15-S-30®, and Rosemary oil.

Another embodiment comprises cyclosporin A, Tergitol 15-S-40®, and Rosemary oil.

Another embodiment comprises cyclosporin A, Tergitol 15-S-40 (70AQ)®, and Rosemary oil.

Another embodiment comprises cyclosporin A, Tergitol 15-S-5®, and Rosemary oil.

Another embodiment comprises cyclosporin A, Tergitol 15-S-7®, and Rosemary oil.

Another embodiment comprises cyclosporin A, Tergitol 15-S-9®, and Rosemary oil.

Another embodiment comprises cyclosporin A, Tergitol L-101®, and Rosemary oil.

Another embodiment comprises cyclosporin A, Tergitol L-61®, and Rosemary oil.

Another embodiment comprises cyclosporin A, Tergitol L-62®, and Rosemary oil.

Another embodiment comprises cyclosporin A, Tergitol L-64®, and Rosemary oil.

Another embodiment comprises cyclosporin A, Tergitol L-81®, and Rosemary oil.

Another embodiment comprises cyclosporin A, Tergitol NP-10®, and Rosemary oil.

Another embodiment comprises cyclosporin A, Tergitol NP-11®, and Rosemary oil.

Another embodiment comprises cyclosporin A, Tergitol NP-12®, and Rosemary oil.

Another embodiment comprises cyclosporin A, Tergitol NP-13®, and Rosemary oil.

Another embodiment comprises cyclosporin A, Tergitol NP-15®, and Rosemary oil.

Another embodiment comprises cyclosporin A, Tergitol NP-30®, and Rosemary oil.

Another embodiment comprises cyclosporin A, Tergitol NP-30 (70 AQ)®, and Rosemary oil.

Another embodiment comprises cyclosporin A, Tergitol NP-4®, and Rosemary oil.

Another embodiment comprises cyclosporin A, Tergitol NP-40®, and Rosemary oil.

Another embodiment comprises cyclosporin A, Tergitol NP-40 (70 AQ)®, and Rosemary oil.

Another embodiment comprises cyclosporin A, Tergitol NP-50 (70 AQ)®, and Rosemary oil.

Another embodiment comprises cyclosporin A, Tergitol NP-55 (70 AQ)®, and Rosemary oil.

Another embodiment comprises cyclosporin A, Tergitol NP-6®, and Rosemary oil.

Another embodiment comprises cyclosporin A, Tergitol NP-7®, and Rosemary oil.

Another embodiment comprises cyclosporin A, Tergitol NP-70 (70 AQ)®, and Rosemary oil.

Another embodiment comprises cyclosporin A, Tergitol NP-8®, and Rosemary oil.

Another embodiment comprises cyclosporin A, Tergitol NP-9®, and Rosemary oil.

Another embodiment comprises cyclosporin A, Tergitol NP-9.5®, and Rosemary oil.

Another embodiment comprises cyclosporin A, Tergitol TMN-10®, and Rosemary oil.

Another embodiment comprises cyclosporin A, Tergitol TMN-3®, and Rosemary oil.

Another embodiment comprises cyclosporin A, Tergitol TMN-6®, and Rosemary oil.

Another embodiment comprises cyclosporin A, Tergitol XD®, and Rosemary oil.

Another embodiment comprises cyclosporin A, Tergitol XH®, and Rosemary oil.

Another embodiment comprises cyclosporin A, Tergitol XJ®, and Rosemary oil.

Another embodiment comprises cyclosporin A, Tetronic 1107®, and Rosemary oil.

Another embodiment comprises cyclosporin A, Tetronic 1307®, and Rosemary oil.

Another embodiment comprises cyclosporin A, Tetronic 150R1®, and Rosemary oil.

Another embodiment comprises cyclosporin A, Tetronic 304®, and Rosemary oil.

Another embodiment comprises cyclosporin A, Tetronic 701®, and Rosemary oil.

Another embodiment comprises cyclosporin A, Tetronic 901®, and Rosemary oil.

Another embodiment comprises cyclosporin A, Tetronic 904®, and Rosemary oil.

Another embodiment comprises cyclosporin A, Tetronic 908®, and Rosemary oil.

Another embodiment comprises cyclosporin A, Tetronic 90R4®, and Rosemary oil.

Another embodiment comprises cyclosporin A, Tomadol 1-3®, and Rosemary oil.

Another embodiment comprises cyclosporin A, Tomadol 1-5®, and Rosemary oil.

Another embodiment comprises cyclosporin A, Tomadol 1-7®, and Rosemary oil.

Another embodiment comprises cyclosporin A, Tomadol 23-1®, and Rosemary oil.

Another embodiment comprises cyclosporin A, Tomadol 23-3®, and Rosemary oil.

Another embodiment comprises cyclosporin A, Tomadol 23-5®, and Rosemary oil.

Another embodiment comprises cyclosporin A, Tomadol 23-6.5®, and Rosemary oil.

Another embodiment comprises cyclosporin A, Tomadol 25-12®, and Rosemary oil.

Another embodiment comprises cyclosporin A, Tomadol 25-3®, and Rosemary oil.

Another embodiment comprises cyclosporin A, Tomadol 25-7®, and Rosemary oil.

Another embodiment comprises cyclosporin A, Tomadol 25-9®, and Rosemary oil.

Another embodiment comprises cyclosporin A, Tomadol 45-13®, and Rosemary oil.

Another embodiment comprises cyclosporin A, Tomadol 45-2.5®, and Rosemary oil.

Another embodiment comprises cyclosporin A, Tomadol 45-7®, and Rosemary oil.

Another embodiment comprises cyclosporin A, Tomadol 91-2.5®, and Rosemary oil.

Another embodiment comprises cyclosporin A, Tomadol 91-6®, and Rosemary oil.

Another embodiment comprises cyclosporin A, Tomadol 91-8®, and Rosemary oil.

Another embodiment comprises cyclosporin A, Triton X-100®, and Rosemary oil.

Another embodiment comprises cyclosporin A, Triton X-102®, and Rosemary oil.

Another embodiment comprises cyclosporin A, Triton X-114®, and Rosemary oil.

Another embodiment comprises cyclosporin A, Triton X-15®, and Rosemary oil.

Another embodiment comprises cyclosporin A, Triton X-165 (70%)®, and Rosemary oil.

Another embodiment comprises cyclosporin A, Triton X-207®, and Rosemary oil.

Another embodiment comprises cyclosporin A, Triton X-305 (70%)®, and Rosemary oil.

Another embodiment comprises cyclosporin A, Triton X-35®, and Rosemary oil.

Another embodiment comprises cyclosporin A, Triton X-405 (70%)®, and Rosemary oil.

Another embodiment comprises cyclosporin A, Triton X-45®, and Rosemary oil.

Another embodiment comprises cyclosporin A, Triton X-705 (70%)®, and Rosemary oil.

Another embodiment comprises cyclosporin A, Volpo S-10®, and Rosemary oil.

Another embodiment comprises cyclosporin A, Volpo S-2®, and Rosemary oil.

Another embodiment comprises cyclosporin A, Volpo S-20®, and Rosemary oil.

Another embodiment comprises cyclosporin A, Volpo-10®, and Rosemary oil.

Another embodiment comprises cyclosporin A, Volpo-20®, and Rosemary oil.

Another embodiment comprises cyclosporin A, Volpo-3®, and Rosemary oil.

Another embodiment comprises cyclosporin A, Volpo-5®, and Rosemary oil.

Another embodiment comprises cyclosporin A, Abex 2515®, and Peanut oil.

Another embodiment comprises cyclosporin A, Abex 2525/40®, and Peanut oil.

Another embodiment comprises cyclosporin A, Abex 2535®, and Peanut oil.

Another embodiment comprises cyclosporin A, Abex 2545®, and Peanut oil.

Another embodiment comprises cyclosporin A, Acconon E®, and Peanut oil.

Another embodiment comprises cyclosporin A, Alfonic 1216CO-9®, and Peanut oil.

Another embodiment comprises cyclosporin A, Alfonic 1412-3®, and Peanut oil.

Another embodiment comprises cyclosporin A, Alfonic TDA-12®, and Peanut oil.

Another embodiment comprises cyclosporin A, Alfonic TDA-3®, and Peanut oil.

Another embodiment comprises cyclosporin A, Alfonic TDA-4®, and Peanut oil.

Another embodiment comprises cyclosporin A, Alfonic TDA-6®, and Peanut oil.

Another embodiment comprises cyclosporin A, Alfonic TDA-7®, and Peanut oil.

Another embodiment comprises cyclosporin A, Alfonic TDA-8®, and Peanut oil.

Another embodiment comprises cyclosporin A, Alfonic TDA-9®, and Peanut oil.

Another embodiment comprises cyclosporin A, Alkamuls 400-DC®, and Peanut oil.

Another embodiment comprises cyclosporin A, Alkamuls 400-MO/E®, and Peanut oil.

Another embodiment comprises cyclosporin A, Ameroxol OE-10®, and Peanut oil.

Another embodiment comprises cyclosporin A, Ameroxol OE-2®, and Peanut oil.

Another embodiment comprises cyclosporin A, Ameroxol OE-20®, and Peanut oil.

Another embodiment comprises cyclosporin A, Armix 176®, and Peanut oil.

Another embodiment comprises cyclosporin A, Armix 180-C®, and Peanut oil.

Another embodiment comprises cyclosporin A, Armix 183®, and Peanut oil.

Another embodiment comprises cyclosporin A, Brij 35®, and Peanut oil.

Another embodiment comprises cyclosporin A, Brij 35 Liquid®, and Peanut oil.

Another embodiment comprises cyclosporin A, Brij 35 SP®, and Peanut oil.

Another embodiment comprises cyclosporin A, Canasol MJ 2109®, and Peanut oil.

Another embodiment comprises cyclosporin A, Canasol MJ 52®, and Peanut oil.

Another embodiment comprises cyclosporin A, Canasol R 3600®, and Peanut oil.

Another embodiment comprises cyclosporin A, Canasol R 3603®, and Peanut oil.

Another embodiment comprises cyclosporin A, Canasol R 4000 H®, and Peanut oil.

Another embodiment comprises cyclosporin A, Cedepal CA-210®, and Peanut oil.

Another embodiment comprises cyclosporin A, Cedepal CA-520®, and Peanut oil.

Another embodiment comprises cyclosporin A, Cedepal CA-630®, and Peanut oil.

Another embodiment comprises cyclosporin A, Cedepal CA-720®, and Peanut oil.

Another embodiment comprises cyclosporin A, Cedepal CA-890®, and Peanut oil.

Another embodiment comprises cyclosporin A, Cedepal CA-897®, and Peanut oil.

Another embodiment comprises cyclosporin A, Cedepal CO-210®, and Peanut oil.

Another embodiment comprises cyclosporin A, Cedepal CO-430®, and Peanut oil.

Another embodiment comprises cyclosporin A, Cedepal CO-530®, and Peanut oil.

Another embodiment comprises cyclosporin A, Cedepal CO-630®, and Peanut oil.

Another embodiment comprises cyclosporin A, Cedepal CO-710®, and Peanut oil.

Another embodiment comprises cyclosporin A, Cedepal CO-730®, and Peanut oil.

Another embodiment comprises cyclosporin A, Cedepal CO-880®, and Peanut oil.

Another embodiment comprises cyclosporin A, Cedepal CO-887®, and Peanut oil.

Another embodiment comprises cyclosporin A, Cedepal CO-890®, and Peanut oil.

Another embodiment comprises cyclosporin A, Cedepal CO-897®, and Peanut oil.

Another embodiment comprises cyclosporin A, Cedepal CO-970®, and Peanut oil.

Another embodiment comprises cyclosporin A, Cedepal CO-977®, and Peanut oil.

Another embodiment comprises cyclosporin A, Cedepal CO-990®, and Peanut oil.

Another embodiment comprises cyclosporin A, Cedepal CO-997®, and Peanut oil.

Another embodiment comprises cyclosporin A, Chemal BP 261®, and Peanut oil.

Another embodiment comprises cyclosporin A, Chemal BP 262®, and Peanut oil.

Another embodiment comprises cyclosporin A, Chemal BP 3172®, and Peanut oil.

Another embodiment comprises cyclosporin A, Chemal BP 3174®, and Peanut oil.

Another embodiment comprises cyclosporin A, Chemal DA-4®, and Peanut oil.

Another embodiment comprises cyclosporin A, Chemal DA-6®, and Peanut oil.

Another embodiment comprises cyclosporin A, Chemal DA-9®, and Peanut oil.

Another embodiment comprises cyclosporin A, Chemal LA-23®, and Peanut oil.

Another embodiment comprises cyclosporin A, Chemal LA-4®, and Peanut oil.

Another embodiment comprises cyclosporin A, Chemal LA-9®, and Peanut oil.

Another embodiment comprises cyclosporin A, Chemal OA-20G®, and Peanut oil.

Another embodiment comprises cyclosporin A, Chemal OA-5®, and Peanut oil.

Another embodiment comprises cyclosporin A, Chemal OA-9®, and Peanut oil.

Another embodiment comprises cyclosporin A, Chemal TDA-12®, and Peanut oil.

Another embodiment comprises cyclosporin A, Chemal TDA-15®, and Peanut oil.

Another embodiment comprises cyclosporin A, Chemal TDA-3®, and Peanut oil.

Another embodiment comprises cyclosporin A, Chemal TDA-6®, and Peanut oil.

Another embodiment comprises cyclosporin A, Chemal TDA-9®, and Peanut oil.

Another embodiment comprises cyclosporin A, Chemax CO-16®, and Peanut oil.

Another embodiment comprises cyclosporin A, Chemax CO-200/50®, and Peanut oil.

Another embodiment comprises cyclosporin A, Chemax CO-25®, and Peanut oil.

Another embodiment comprises cyclosporin A, Chemax CO-30®, and Peanut oil.

Another embodiment comprises cyclosporin A, Chemax CO-36®, and Peanut oil.

Another embodiment comprises cyclosporin A, Chemax CO-40®, and Peanut oil.

Another embodiment comprises cyclosporin A, Chemax CO-5®, and Peanut oil.

Another embodiment comprises cyclosporin A, Chemax CO-80®, and Peanut oil.

Another embodiment comprises cyclosporin A, Chemax DNP-150/50®, and Peanut oil.

Another embodiment comprises cyclosporin A, Chemax DNP-18®, and Peanut oil.

Another embodiment comprises cyclosporin A, Chemax DNP-8®, and Peanut oil.

Another embodiment comprises cyclosporin A, Chemax E 200 ML®, and Peanut oil.

Another embodiment comprises cyclosporin A, Chemax E 200 MO®, and Peanut oil.

Another embodiment comprises cyclosporin A, Chemax E 400 ML®, and Peanut oil.

Another embodiment comprises cyclosporin A, Chemax E 400 MO®, and Peanut oil.

Another embodiment comprises cyclosporin A, Chemax E 600 ML®, and Peanut oil.

Another embodiment comprises cyclosporin A, Chemax E 600 MO®, and Peanut oil.

Another embodiment comprises cyclosporin A, Chemax E 1000 MO®, and Peanut oil.

Another embodiment comprises cyclosporin A, Chemax E 1000 MS®, and Peanut oil.

Another embodiment comprises cyclosporin A, Chemax E200 MS®, and Peanut oil.

Another embodiment comprises cyclosporin A, Chemax E400 MS®, and Peanut oil.

Another embodiment comprises cyclosporin A, Chemax E600 MS®, and Peanut oil.

Another embodiment comprises cyclosporin A, Chemax HCO-16®, and Peanut oil.

Another embodiment comprises cyclosporin A, Chemax HCO-200/50®, and Peanut oil.

Another embodiment comprises cyclosporin A, Chemax HCO-25®, and Peanut oil.

Another embodiment comprises cyclosporin A, Chemax HCO-5®, and Peanut oil.

Another embodiment comprises cyclosporin A, Chemax TO-16®, and Peanut oil.

Another embodiment comprises cyclosporin A, Chemax TO-8®, and Peanut oil.

Another embodiment comprises cyclosporin A, Colalube 3409®, and Peanut oil.

Another embodiment comprises cyclosporin A, Colalube 3411®, and Peanut oil.

Another embodiment comprises cyclosporin A, Colalube 3413®, and Peanut oil.

Another embodiment comprises cyclosporin A, Colalube 3414®, and Peanut oil.

Another embodiment comprises cyclosporin A, Colalube 3415®, and Peanut oil.

Another embodiment comprises cyclosporin A, Colalube 3416®, and Peanut oil.

Another embodiment comprises cyclosporin A, Colalube 3417®, and Peanut oil.

Another embodiment comprises cyclosporin A, Delonic NPE-100®, and Peanut oil.

Another embodiment comprises cyclosporin A, Delonic NPE-30®, and Peanut oil.

Another embodiment comprises cyclosporin A, Delonic NPE-40®, and Peanut oil.

Another embodiment comprises cyclosporin A, Delonic OPE-10®, and Peanut oil.

Another embodiment comprises cyclosporin A, Delonic OPE-12®, and Peanut oil.

Another embodiment comprises cyclosporin A, Delonic OPE-30®, and Peanut oil.

Another embodiment comprises cyclosporin A, Delonic OPE-40®, and Peanut oil.

Another embodiment comprises cyclosporin A, Delonic OPE-5®, and Peanut oil.

Another embodiment comprises cyclosporin A, Delonic OPE-7.5®, and Peanut oil.

Another embodiment comprises cyclosporin A, DePeg 16-CO®, and Peanut oil.

Another embodiment comprises cyclosporin A, DePeg 25-CO®, and Peanut oil.

Another embodiment comprises cyclosporin A, DePeg 30-CO®, and Peanut oil.

Another embodiment comprises cyclosporin A, DePeg 40-CO®, and Peanut oil.

Another embodiment comprises cyclosporin A, DePeg 5-CO®, and Peanut oil.

Another embodiment comprises cyclosporin A, DePeg 80-CO®, and Peanut oil.

Another embodiment comprises cyclosporin A, DeSonic S-100®, and Peanut oil.

Another embodiment comprises cyclosporin A, DeSonic S-114®, and Peanut oil.

Another embodiment comprises cyclosporin A, DeSonic S-405®, and Peanut oil.

Another embodiment comprises cyclosporin A, DeSonic S-45®, and Peanut oil.

Another embodiment comprises cyclosporin A, DeSonic TDA-9®, and Peanut oil.

Another embodiment comprises cyclosporin A, DeThox Acid O-14®, and Peanut oil.

Another embodiment comprises cyclosporin A, DeThox Acid O-16®, and Peanut oil.

Another embodiment comprises cyclosporin A, DeThox Acid O-9®, and Peanut oil.

Another embodiment comprises cyclosporin A, DeThox Acid TO-14®, and Peanut oil.

Another embodiment comprises cyclosporin A, DeThox Acid TO-16.5®, and Peanut oil.

Another embodiment comprises cyclosporin A, DeThox Acid TO-8.5®, and Peanut oil.

Another embodiment comprises cyclosporin A, Dur-Em 114®, and Peanut oil.

Another embodiment comprises cyclosporin A, Dur-Em 117®, and Peanut oil.

Another embodiment comprises cyclosporin A, Dur-Em 204®, and Peanut oil.

Another embodiment comprises cyclosporin A, Dur-Em 207®, and Peanut oil.

Another embodiment comprises cyclosporin A, Dur-Em 207E®, and Peanut oil.

Another embodiment comprises cyclosporin A, Emerest 2704®, and Peanut oil.

Another embodiment comprises cyclosporin A, Emerest 2712®, and Peanut oil.

Another embodiment comprises cyclosporin A, Emerest 2715®, and Peanut oil.

Another embodiment comprises cyclosporin A, Ethal EH-2®, and Peanut oil.

Another embodiment comprises cyclosporin A, Ethal EH-5®, and Peanut oil.

Another embodiment comprises cyclosporin A, Ethal OA-23®, and Peanut oil.

Another embodiment comprises cyclosporin A, Ethosperse CA-20®, and Peanut oil.

Another embodiment comprises cyclosporin A, Ethosperse LA-12®, and Peanut oil.

Another embodiment comprises cyclosporin A, Ethosperse LA-23®, and Peanut oil.

Another embodiment comprises cyclosporin A, Ethosperse LA-4®, and Peanut oil.

Another embodiment comprises cyclosporin A, Ethox 3095®, and Peanut oil.

Another embodiment comprises cyclosporin A, Ethox CO-16®, and Peanut oil.

Another embodiment comprises cyclosporin A, Ethox CO-200®, and Peanut oil.

Another embodiment comprises cyclosporin A, Ethox CO-25®, and Peanut oil.

Another embodiment comprises cyclosporin A, Ethox CO-30®, and Peanut oil.

Another embodiment comprises cyclosporin A, Ethox CO-36®, and Peanut oil.

Another embodiment comprises cyclosporin A, Ethox CO-40®, and Peanut oil.

Another embodiment comprises cyclosporin A, Ethox CO-5®, and Peanut oil.

Another embodiment comprises cyclosporin A, Ethox DO-14®, and Peanut oil.

Another embodiment comprises cyclosporin A, Ethox DO-9®, and Peanut oil.

Another embodiment comprises cyclosporin A, Ethox MA-8®, and Peanut oil.

Another embodiment comprises cyclosporin A, Ethox MI-14®, and Peanut oil.

Another embodiment comprises cyclosporin A, Ethox ML-14®, and Peanut oil.

Another embodiment comprises cyclosporin A, Ethox ML-5®, and Peanut oil.

Another embodiment comprises cyclosporin A, Ethox MO-14®, and Peanut oil.

Another embodiment comprises cyclosporin A, Ethox MO-9®, and Peanut oil.

Another embodiment comprises cyclosporin A, Ethox MS-14®, and Peanut oil.

Another embodiment comprises cyclosporin A, Ethox MS-23®, and Peanut oil.

Another embodiment comprises cyclosporin A, Ethox MS-40®, and Peanut oil.

Another embodiment comprises cyclosporin A, Ethox MS-8®, and Peanut oil.

Another embodiment comprises cyclosporin A, Ethox TO-16, and Peanut oil.

Another embodiment comprises cyclosporin A, Ethox TO-8®, and Peanut oil.

Another embodiment comprises cyclosporin A, Ethylan SN-120®, and Peanut oil.

Another embodiment comprises cyclosporin A, Ethylan SN-70®, and Peanut oil.

Another embodiment comprises cyclosporin A, Ethylan SN-90®, and Peanut oil.

Another embodiment comprises cyclosporin A, Flexricin 13®, and Peanut oil.

Another embodiment comprises cyclosporin A, Flexricin 15®, and Peanut oil.

Another embodiment comprises cyclosporin A, Genapol 1261®, and Peanut oil.

Another embodiment comprises cyclosporin A, Generol 122 E25®, and Peanut oil.

Another embodiment comprises cyclosporin A, Glicepol 160®, and Peanut oil.

Another embodiment comprises cyclosporin A, Glicepol 560®, and Peanut oil.

Another embodiment comprises cyclosporin A, Glicepol GMS 20®, and Peanut oil.

Another embodiment comprises cyclosporin A, Hetoxide BN-13®, and Peanut oil.

Another embodiment comprises cyclosporin A, Hetoxide DNP-4®, and Peanut oil.

Another embodiment comprises cyclosporin A, Hetoxide DNP-9.69®, and Peanut oil.

Another embodiment comprises cyclosporin A, Hetoxide G-268®, and Peanut oil.

Another embodiment comprises cyclosporin A, Hetoxide G-7®, and Peanut oil.

Another embodiment comprises cyclosporin A, Hetoxide GT-20®, and Peanut oil.

Another embodiment comprises cyclosporin A, Hetoxide HC-40®, and Peanut oil.

Another embodiment comprises cyclosporin A, Hetoxol C-24®, and Peanut oil.

Another embodiment comprises cyclosporin A, Hetoxol CA-10®, and Peanut oil.

Another embodiment comprises cyclosporin A, Hetoxol CA-2®, and Peanut oil.

Another embodiment comprises cyclosporin A, Hetoxol CA-20®, and Peanut oil.

Another embodiment comprises cyclosporin A, Hetoxol CAWS®, and Peanut oil.

Another embodiment comprises cyclosporin A, Hetoxol CD-3®, and Peanut oil.

Another embodiment comprises cyclosporin A, Hetoxol CD-4®, and Peanut oil.

Another embodiment comprises cyclosporin A, Hetoxol CS-15®, and Peanut oil.

Another embodiment comprises cyclosporin A, Hetoxol CS-20®, and Peanut oil.

Another embodiment comprises cyclosporin A, Hetoxol CS-20D®, and Peanut oil.

Another embodiment comprises cyclosporin A, Hetoxol CS-25®, and Peanut oil.

Another embodiment comprises cyclosporin A, Hetoxol CS-50®, and Peanut oil.

Another embodiment comprises cyclosporin A, Hetoxol CS-9®, and Peanut oil.

Another embodiment comprises cyclosporin A, Hetoxol CSA-15®, and Peanut oil.

Another embodiment comprises cyclosporin A, Hetoxol D®, and Peanut oil.

Another embodiment comprises cyclosporin A, Hetoxol G®, and Peanut oil.

Another embodiment comprises cyclosporin A, Hetoxol J®, and Peanut oil.

Another embodiment comprises cyclosporin A, Hetoxol L®, and Peanut oil.

Another embodiment comprises cyclosporin A, Hetoxol L-23®, and Peanut oil.

Another embodiment comprises cyclosporin A, Hetoxol L-4®, and Peanut oil.

Another embodiment comprises cyclosporin A, Hetoxol L-9®, and Peanut oil.

Another embodiment comprises cyclosporin A, Hetoxol LS-9®, and Peanut oil.

Another embodiment comprises cyclosporin A, Hetoxol M-3®, and Peanut oil.

Another embodiment comprises cyclosporin A, Hetoxol OA-10 Special®, and Peanut oil.

Another embodiment comprises cyclosporin A, Hetoxol OA-20 Special®, and Peanut oil.

Another embodiment comprises cyclosporin A, Hetoxol OA-3 Special®, and Peanut oil.

Another embodiment comprises cyclosporin A, Hetoxol OA-5 Special®, and Peanut oil.

Another embodiment comprises cyclosporin A, Hetoxol OL-10®, and Peanut oil.

Another embodiment comprises cyclosporin A, Hetoxol OL-2®, and Peanut oil.

Another embodiment comprises cyclosporin A, Hetoxol OL-23®, and Peanut oil.

Another embodiment comprises cyclosporin A, Hetoxol OL-4®, and Peanut oil.

Another embodiment comprises cyclosporin A, Hetoxol OL-40®, and Peanut oil.

Another embodiment comprises cyclosporin A, Hetoxol OL-5®, and Peanut oil.

Another embodiment comprises cyclosporin A, Hetoxol PLA®, and Peanut oil.

Another embodiment comprises cyclosporin A, Hetoxol SP-15®, and Peanut oil.

Another embodiment comprises cyclosporin A, Hetoxol STA-10®, and Peanut oil.

Another embodiment comprises cyclosporin A, Hetoxol STA-2®, and Peanut oil.

Another embodiment comprises cyclosporin A, Hetoxol STA-30®, and Peanut oil.

Another embodiment comprises cyclosporin A, Hetoxol TD-12®, and Peanut oil.

Another embodiment comprises cyclosporin A, Hetoxol TD-18®, and Peanut oil.

Another embodiment comprises cyclosporin A, Hetoxol TD-3®, and Peanut oil.

Another embodiment comprises cyclosporin A, Hetoxol TD-6®, and Peanut oil.

Another embodiment comprises cyclosporin A, Hetoxol TD-9®, and Peanut oil.

Another embodiment comprises cyclosporin A, Hetoxol TDEP-15®, and Peanut oil.

Another embodiment comprises cyclosporin A, Hetoxol TDEP-63®, and Peanut oil.

Another embodiment comprises cyclosporin A, Iconol 24-12®, and Peanut oil.

Another embodiment comprises cyclosporin A, Iconol 24-23®, and Peanut oil.

Another embodiment comprises cyclosporin A, Iconol 24-4®, and Peanut oil.

Another embodiment comprises cyclosporin A, Iconol DA-4®, and Peanut oil.

Another embodiment comprises cyclosporin A, Iconol DA-6®, and Peanut oil.

Another embodiment comprises cyclosporin A, Iconol TDA-10®, and Peanut oil.

Another embodiment comprises cyclosporin A, Iconol TDA-3®, and Peanut oil.

Another embodiment comprises cyclosporin A, Iconol TDA-6®, and Peanut oil.

Another embodiment comprises cyclosporin A, Iconol TDA-8-90%®, and Peanut oil.

Another embodiment comprises cyclosporin A, Iconol TDA-9®, and Peanut oil.

Another embodiment comprises cyclosporin A, Igepal CA-210®, and Peanut oil.

Another embodiment comprises cyclosporin A, Igepal CA407®, and Peanut oil.

Another embodiment comprises cyclosporin A, Igepal CA-420®, and Peanut oil.

Another embodiment comprises cyclosporin A, Igepal CA-520®, and Peanut oil.

Another embodiment comprises cyclosporin A, Igepal CA620®, and Peanut oil.

Another embodiment comprises cyclosporin A, Igepal CA-630®, and Peanut oil.

Another embodiment comprises cyclosporin A, Igepal CA-720®, and Peanut oil.

Another embodiment comprises cyclosporin A, Igepal CA-877®, and Peanut oil.

Another embodiment comprises cyclosporin A, Igepal CA-887®, and Peanut oil.

Another embodiment comprises cyclosporin A, Igepal CA-897®, and Peanut oil.

Another embodiment comprises cyclosporin A, Igepal CO-210®, and Peanut oil.

Another embodiment comprises cyclosporin A, Igepal CO-430®, and Peanut oil.

Another embodiment comprises cyclosporin A, Igepal CO-520®, and Peanut oil.

Another embodiment comprises cyclosporin A, Igepal CO-530®, and Peanut oil.

Another embodiment comprises cyclosporin A, Igepal CO-610®, and Peanut oil.

Another embodiment comprises cyclosporin A, Igepal CO-630®, and Peanut oil.

Another embodiment comprises cyclosporin A, Igepal CO-630 Special®, and Peanut oil.

Another embodiment comprises cyclosporin A, Igepal CO-660®, and Peanut oil.

Another embodiment comprises cyclosporin A, Igepal CO-710®, and Peanut oil.

Another embodiment comprises cyclosporin A, Igepal CO-720®, and Peanut oil.

Another embodiment comprises cyclosporin A, Igepal CO-730®, and Peanut oil.

Another embodiment comprises cyclosporin A, Igepal CO-738®, and Peanut oil.

Another embodiment comprises cyclosporin A, Igepal CO-850®, and Peanut oil.

Another embodiment comprises cyclosporin A, Igepal CO-880®, and Peanut oil.

Another embodiment comprises cyclosporin A, Igepal CO-887®, and Peanut oil.

Another embodiment comprises cyclosporin A, Igepal CO-890®, and Peanut oil.

Another embodiment comprises cyclosporin A, Igepal CO-897®, and Peanut oil.

Another embodiment comprises cyclosporin A, Igepal CO-970®, and Peanut oil.

Another embodiment comprises cyclosporin A, Igepal CO-977®, and Peanut oil.

Another embodiment comprises cyclosporin A, Igepal CO-987®, and Peanut oil.

Another embodiment comprises cyclosporin A, Igepal CO-990 FLK®, and Peanut oil.

Another embodiment comprises cyclosporin A, Igepal CO-997®, and Peanut oil.

Another embodiment comprises cyclosporin A, Igepal DM-430®, and Peanut oil.

Another embodiment comprises cyclosporin A, Igepal DM-530®, and Peanut oil.

Another embodiment comprises cyclosporin A, Igepal DM-710®, and Peanut oil.

Another embodiment comprises cyclosporin A, Igepal DM-730®, and Peanut oil.

Another embodiment comprises cyclosporin A, Igepal DM-880®, and Peanut oil.

Another embodiment comprises cyclosporin A, Igepal DM-970 FLK®, and Peanut oil.

Another embodiment comprises cyclosporin A, Igepal OD-410®, and Peanut oil.

Another embodiment comprises cyclosporin A, Igepal RC-520®, and Peanut oil.

Another embodiment comprises cyclosporin A, Igepal RC-620®, and Peanut oil.

Another embodiment comprises cyclosporin A, Igepal RC-630®, and Peanut oil.

Another embodiment comprises cyclosporin A, Lexemul CS-20®, and Peanut oil.

Another embodiment comprises cyclosporin A, Lipo GMS 450®, and Peanut oil.

Another embodiment comprises cyclosporin A, Lipo Polyglycol 1000®, and Peanut oil.

Another embodiment comprises cyclosporin A, Lipo Polyglycol 200®, and Peanut oil.

Another embodiment comprises cyclosporin A, Lipo Polyglycol 300®, and Peanut oil.

Another embodiment comprises cyclosporin A, Lipo Polyglycol 3350®, and Peanut oil.

Another embodiment comprises cyclosporin A, Lipo Polyglycol 400®, and Peanut oil.

Another embodiment comprises cyclosporin A, Lipo Polyglycol 600®, and Peanut oil.

Another embodiment comprises cyclosporin A, Lipocol C-10®, and Peanut oil.

Another embodiment comprises cyclosporin A, Lipocol C-2®, and Peanut oil.

Another embodiment comprises cyclosporin A, Lipocol C-20®, and Peanut oil.

Another embodiment comprises cyclosporin A, Lipocol L-12®, and Peanut oil.

Another embodiment comprises cyclosporin A, Lipocol L-23®, and Peanut oil.

Another embodiment comprises cyclosporin A, Lipocol L-4®, and Peanut oil.

Another embodiment comprises cyclosporin A, Lipocol O-10®, and Peanut oil.

Another embodiment comprises cyclosporin A, Lipocol O-2®, and Peanut oil.

Another embodiment comprises cyclosporin A, Lipocol O-20®, and Peanut oil.

Another embodiment comprises cyclosporin A, Lipocol O-5®, and Peanut oil.

Another embodiment comprises cyclosporin A, Lipocol S-10®, and Peanut oil.

Another embodiment comprises cyclosporin A, Lipocol S-2®, and Peanut oil.

Another embodiment comprises cyclosporin A, Lipocol S-20®, and Peanut oil.

Another embodiment comprises cyclosporin A, Lipocol SC-15®, and Peanut oil.

Another embodiment comprises cyclosporin A, Lipocol SC-20®, and Peanut oil.

Another embodiment comprises cyclosporin A, Lipocol SC-4®, and Peanut oil.

Another embodiment comprises cyclosporin A, Lipocol TD-12®, and Peanut oil.

Another embodiment comprises cyclosporin A, Lumulse 40-O K®, and Peanut oil.

Another embodiment comprises cyclosporin A, Lumulse 42-O K®, and Peanut oil.

Another embodiment comprises cyclosporin A, Lumulse GMT-40®, and Peanut oil.

Another embodiment comprises cyclosporin A, Lumulse PEG 1450®, and Peanut oil.

Another embodiment comprises cyclosporin A, Lumulse PEG 1450 NF®, and Peanut oil.

Another embodiment comprises cyclosporin A, Lumulse PEG 8000®, and Peanut oil.

Another embodiment comprises cyclosporin A, Lumulse PGO®, and Peanut oil.

Another embodiment comprises cyclosporin A, Lumulse POE (2) Oleyl Amine®, and Peanut oil.

Another embodiment comprises cyclosporin A, Magrabar PGO-104®, and Peanut oil.

Another embodiment comprises cyclosporin A, Mapeg 200 ML®, and Peanut oil.

Another embodiment comprises cyclosporin A, Mapeg 400 MO®, and Peanut oil.

Another embodiment comprises cyclosporin A, Mapeg 400 MOT®, and Peanut oil.

Another embodiment comprises cyclosporin A, Mapeg 400DO®, and Peanut oil.

Another embodiment comprises cyclosporin A, Mapeg 400DOT®, and Peanut oil.

Another embodiment comprises cyclosporin A, Mapeg 600DOT®, and Peanut oil.

Another embodiment comprises cyclosporin A, Marlox B 24/80®, and Peanut oil.

Another embodiment comprises cyclosporin A, Marlox FK 86®, and Peanut oil.

Another embodiment comprises cyclosporin A, Marlox MO 154®, and Peanut oil.

Another embodiment comprises cyclosporin A, Mazox CDA CG®, and Peanut oil.

Another embodiment comprises cyclosporin A, Monoglycerides Glyceryl Monestearate®, and Peanut oil.

Another embodiment comprises cyclosporin A, Myrj 45®, and Peanut oil.

Another embodiment comprises cyclosporin A, Myrj 52®, and Peanut oil.

Another embodiment comprises cyclosporin A, Myrj 52S®, and Peanut oil.

Another embodiment comprises cyclosporin A, Myrj 53®, and Peanut oil.

Another embodiment comprises cyclosporin A, Myrj 59®, and Peanut oil.

Another embodiment comprises cyclosporin A, Nortox NP-12®, and Peanut oil.

Another embodiment comprises cyclosporin A, Norfox NP-4®, and Peanut oil.

Another embodiment comprises cyclosporin A, Norfox NP-6®, and Peanut oil.

Another embodiment comprises cyclosporin A, Nortox NP-887®, and Peanut oil.

Another embodiment comprises cyclosporin A, Nortox NP-9®, and Peanut oil.

Another embodiment comprises cyclosporin A, Nortox NP-977®, and Peanut oil.

Another embodiment comprises cyclosporin A, Nortox OP-100®, and Peanut oil.

Another embodiment comprises cyclosporin A, Nortox OP-102®, and Peanut oil.

Another embodiment comprises cyclosporin A, Nortox OP-114®, and Peanut oil.

Another embodiment comprises cyclosporin A, Norfox OP-45®, and Peanut oil.

Another embodiment comprises cyclosporin A, Pegosperse 100-L®, and Peanut oil.

Another embodiment comprises cyclosporin A, Pegosperse 100-O®, and Peanut oil.

Another embodiment comprises cyclosporin A, Pegosperse 100-S®, and Peanut oil.

Another embodiment comprises cyclosporin A, Pegosperse 1500-MS®, and Peanut oil.

Another embodiment comprises cyclosporin A, Pegosperse 1750-MS®, and Peanut oil.

Another embodiment comprises cyclosporin A, Pegosperse 200 DL®, and Peanut oil.

Another embodiment comprises cyclosporin A, Pegosperse 200-ML®, and Peanut oil.

Another embodiment comprises cyclosporin A, Pegosperse 400-DL®, and Peanut oil.

Another embodiment comprises cyclosporin A, Pegosperse 400-DO®, and Peanut oil.

Another embodiment comprises cyclosporin A, Pegosperse 400-DS®, and Peanut oil.

Another embodiment comprises cyclosporin A, Pegosperse 400-ML®, and Peanut oil.

Another embodiment comprises cyclosporin A, Pegosperse 400-MO®, and Peanut oil.

Another embodiment comprises cyclosporin A, Pegosperse 400-MOT®, and Peanut oil.

Another embodiment comprises cyclosporin A, Pegosperse 400-MS®, and Peanut oil.

Another embodiment comprises cyclosporin A, Pegosperse 50-DS®, and Peanut oil.

Another embodiment comprises cyclosporin A, Pegosperse 50-MS®, and Peanut oil.

Another embodiment comprises cyclosporin A, Pegosperse 600-ML®, and Peanut oil.

Another embodiment comprises cyclosporin A, Pegosperse 600-MS®, and Peanut oil.

Another embodiment comprises cyclosporin A, Plurafac A-38®, and Peanut oil.

Another embodiment comprises cyclosporin A, Plurafac B-25-5®, and Peanut oil.

Another embodiment comprises cyclosporin A, Plurafac B-26®, and Peanut oil.

Another embodiment comprises cyclosporin A, Plurafac D-25®, and Peanut oil.

Another embodiment comprises cyclosporin A, Plurafac LF 131®, and Peanut oil.

Another embodiment comprises cyclosporin A, Plurafac LF 4030®, and Peanut oil.

Another embodiment comprises cyclosporin A, Plurafac LF 7000®, and Peanut oil.

Another embodiment comprises cyclosporin A, Plurafac RA-20®, and Peanut oil.

Another embodiment comprises cyclosporin A, Plurafac RA-30®, and Peanut oil.

Another embodiment comprises cyclosporin A, Plurafac RA-40®, and Peanut oil.

Another embodiment comprises cyclosporin A, Pluronic 10R5®, and Peanut oil.

Another embodiment comprises cyclosporin A, Pluronic 17R2®, and Peanut oil.

Another embodiment comprises cyclosporin A, Pluronic 17R4®, and Peanut oil.

Another embodiment comprises cyclosporin A, Pluronic 25R2®, and Peanut oil.

Another embodiment comprises cyclosporin A, Pluronic 25R4®, and Peanut oil.

Another embodiment comprises cyclosporin A, Pluronic 31R1®, and Peanut oil.

Another embodiment comprises cyclosporin A, Pluronic F108®, and Peanut oil.

Another embodiment comprises cyclosporin A, Pluronic F127®, and Peanut oil.

Another embodiment comprises cyclosporin A, Pluronic F38®, and Peanut oil.

Another embodiment comprises cyclosporin A, Pluronic F68®, and Peanut oil.

Another embodiment comprises cyclosporin A, Pluronic F68LF®, and Peanut oil.

Another embodiment comprises cyclosporin A, Pluronic F77®, and Peanut oil.

Another embodiment comprises cyclosporin A, Pluronic F87®, and Peanut oil.

Another embodiment comprises cyclosporin A, Pluronic F88®, and Peanut oil.

Another embodiment comprises cyclosporin A, Pluronic F98®, and Peanut oil.

Another embodiment comprises cyclosporin A, Pluronic L10®, and Peanut oil.

Another embodiment comprises cyclosporin A, Pluronic L101®, and Peanut oil.

Another embodiment comprises cyclosporin A, Pluronic L121®, and Peanut oil.

Another embodiment comprises cyclosporin A, Pluronic L31®, and Peanut oil.

Another embodiment comprises cyclosporin A, Pluronic L35®, and Peanut oil.

Another embodiment comprises cyclosporin A, Pluronic L43®, and Peanut oil.

Another embodiment comprises cyclosporin A, Pluronic L44®, and Peanut oil.

Another embodiment comprises cyclosporin A, Pluronic L61®, and Peanut oil.

Another embodiment comprises cyclosporin A, Pluronic L62®, and Peanut oil.

Another embodiment comprises cyclosporin A, Pluronic L62D®, and Peanut oil.

Another embodiment comprises cyclosporin A, Pluronic L62LF®, and Peanut oil.

Another embodiment comprises cyclosporin A, Pluronic L64®, and Peanut oil.

Another embodiment comprises cyclosporin A, Pluronic L81®, and Peanut oil.

Another embodiment comprises cyclosporin A, Pluronic L92®, and Peanut oil.

Another embodiment comprises cyclosporin A, Pluronic P103®, and Peanut oil.

Another embodiment comprises cyclosporin A, Pluronic P104®, and Peanut oil.

Another embodiment comprises cyclosporin A, Pluronic P105®, and Peanut oil.

Another embodiment comprises cyclosporin A, Pluronic P123®, and Peanut oil.

Another embodiment comprises cyclosporin A, Pluronic P65®, and Peanut oil.

Another embodiment comprises cyclosporin A, Pluronic P84®, and Peanut oil.

Another embodiment comprises cyclosporin A, Pluronic P85®, and Peanut oil.

Another embodiment comprises cyclosporin A, Polyaldo 10-2-P®, and Peanut oil.

Another embodiment comprises cyclosporin A, Polyaldo DGDO®, and Peanut oil.

Another embodiment comprises cyclosporin A, Polyaldo HGDS®, and Peanut oil.

Another embodiment comprises cyclosporin A, Polyaldo TGMS®, and Peanut oil.

Another embodiment comprises cyclosporin A, Protegin V®, and Peanut oil.

Another embodiment comprises cyclosporin A, Protegin W®, and Peanut oil.

Another embodiment comprises cyclosporin A, Protegin WX®, and Peanut oil.

Another embodiment comprises cyclosporin A, Protegin XV®, and Peanut oil.

Another embodiment comprises cyclosporin A, Rhodasurf BC-420®, and Peanut oil.

Another embodiment comprises cyclosporin A, Rhodasurf BC-610®, and Peanut oil.

Another embodiment comprises cyclosporin A, Rhodasurf BC-630®, and Peanut oil.

Another embodiment comprises cyclosporin A, Rhodasurf BC-720®, and Peanut oil.

Another embodiment comprises cyclosporin A, Rhodasurf BC-840®, and Peanut oil.

Another embodiment comprises cyclosporin A, Rhodasurf L 1®, and Peanut oil.

Another embodiment comprises cyclosporin A, Rhodasurf L 12®, and Peanut oil.

Another embodiment comprises cyclosporin A, Rhodasurf L 2®, and Peanut oil.

Another embodiment comprises cyclosporin A, Rhodasurf L 20®, and Peanut oil.

Another embodiment comprises cyclosporin A, Rhodasurf L 3®, and Peanut oil.

Another embodiment comprises cyclosporin A, Rhodasurf L 30®, and Peanut oil.

Another embodiment comprises cyclosporin A, Rhodasurf L 4®, and Peanut oil.

Another embodiment comprises cyclosporin A, Rhodasun L 50®, and Peanut oil.

Another embodiment comprises cyclosporin A, Rhodasurf L 7/90®, and Peanut oil.

Another embodiment comprises cyclosporin A, Rhodasurf LA-3®, and Peanut oil.

Another embodiment comprises cyclosporin A, Rhodasurf LA-7®, and Peanut oil.

Another embodiment comprises cyclosporin A, Rhodasurf LAN-23®, and Peanut oil.

Another embodiment comprises cyclosporin A, Rhodasun ON-870®, and Peanut oil.

Another embodiment comprises cyclosporin A, Rhodasurf ON-877®, and Peanut oil.

Another embodiment comprises cyclosporin A, Rhodasurf TDA-8/5®, and Peanut oil.

Another embodiment comprises cyclosporin A, Ritapro 100®, and Peanut oil.

Another embodiment comprises cyclosporin A, Ritapro 165®, and Peanut oil.

Another embodiment comprises cyclosporin A, Ritapro 200®, and Peanut oil.

Another embodiment comprises cyclosporin A, Ritapro 300®, and Peanut oil.

Another embodiment comprises cyclosporin A, Ritoleth 10®, and Peanut oil.

Another embodiment comprises cyclosporin A, Ritoleth 2®, and Peanut oil.

Another embodiment comprises cyclosporin A, Ritoleth 20®, and Peanut oil.

Another embodiment comprises cyclosporin A, Ritoleth 5®, and Peanut oil.

Another embodiment comprises cyclosporin A, Santone 10-10-0®, and Peanut oil.

Another embodiment comprises cyclosporin A, Santone 3-1-S XTR®, and Peanut oil.

Another embodiment comprises cyclosporin A, Santone 3-1-SH®, and Peanut oil.

Another embodiment comprises cyclosporin A, Santone 8-1-O®, and Peanut oil.

Another embodiment comprises cyclosporin A, Solulan 16®, and Peanut oil.

Another embodiment comprises cyclosporin A, Solulan 75®, and Peanut oil.

Another embodiment comprises cyclosporin A, Solulan C-24®, and Peanut oil.

Another embodiment comprises cyclosporin A, Solulan L-575®, and Peanut oil.

Another embodiment comprises cyclosporin A, Standamox CAW®, and Peanut oil.

Another embodiment comprises cyclosporin A, Surfine AZI-A®, and Peanut oil.

Another embodiment comprises cyclosporin A, Surfonic CO-15®, and Peanut oil.

Another embodiment comprises cyclosporin A, Surfonic CO-25®, and Peanut oil.

Another embodiment comprises cyclosporin A, Surfonic CO-30®, and Peanut oil.

Another embodiment comprises cyclosporin A, Surfonic CO-36®, and Peanut oil.

Another embodiment comprises cyclosporin A, Surfonic CO-42®, and Peanut oil.

Another embodiment comprises cyclosporin A, Surfonic E400-MO®, and Peanut oil.

Another embodiment comprises cyclosporin A, Surfonic L12-3®, and Peanut oil.

Another embodiment comprises cyclosporin A, Surfonic L12-6®, and Peanut oil.

Another embodiment comprises cyclosporin A, Surfonic L12-8®, and Peanut oil.

Another embodiment comprises cyclosporin A, Surfonic I24-12®, and Peanut oil.

Another embodiment comprises cyclosporin A, Surfonic L24-2®, and Peanut oil.

Another embodiment comprises cyclosporin A, Surfonic L24-22®, and Peanut oil.

Another embodiment comprises cyclosporin A, Surfonic L24-3®, and Peanut oil.

Another embodiment comprises cyclosporin A, Surfonic L24-4®, and Peanut oil.

Another embodiment comprises cyclosporin A, Surfonic L24-5®, and Peanut oil.

Another embodiment comprises cyclosporin A, Surfonic L24-7®, and Peanut oil.

Another embodiment comprises cyclosporin A, Surfonic L24-7.1®, and Peanut oil.

Another embodiment comprises cyclosporin A, Surfonic L24-9®, and Peanut oil.

Another embodiment comprises cyclosporin A, Surfonic LF-17®, and Peanut oil.

Another embodiment comprises cyclosporin A, Surfonic LF-18®, and Peanut oil.

Another embodiment comprises cyclosporin A, Surfonic LF-37®, and Peanut oil.

Another embodiment comprises cyclosporin A, Surfonic LF-41®, and Peanut oil.

Another embodiment comprises cyclosporin A, Surfonic LF-42®, and Peanut oil.

Another embodiment comprises cyclosporin A, Surfonic LF-47®, and Peanut oil.

Another embodiment comprises cyclosporin A, Surfonic LF-68®, and Peanut oil.

Another embodiment comprises cyclosporin A, Surfonic N-10®, and Peanut oil.

Another embodiment comprises cyclosporin A, Surfonic N-100®, and Peanut oil.

Another embodiment comprises cyclosporin A, Surfonic N-102®,and Peanut oil.

Another embodiment comprises cyclosporin A, Surfonic N-120®, and Peanut oil.

Another embodiment comprises cyclosporin A, Surfonic N-150®, and Peanut oil.

Another embodiment comprises cyclosporin A, Surfonic N-200®, and Peanut oil.

Another embodiment comprises cyclosporin A, Surfonic N-300®, and Peanut oil.

Another embodiment comprises cyclosporin A, Surfonic N-31.5®, and Peanut oil.

Another embodiment comprises cyclosporin A, Surfonic N-40®, and Peanut oil.

Another embodiment comprises cyclosporin A, Surfonic N-400®, and Peanut oil.

Another embodiment comprises cyclosporin A, Surfonic N-60®, and Peanut oil.

Another embodiment comprises cyclosporin A, Surfonic N-85®, and Peanut oil.

Another embodiment comprises cyclosporin A, Surfonic N-95®, and Peanut oil.

Another embodiment comprises cyclosporin A, Surfonic NB-18®, and Peanut oil.

Another embodiment comprises cyclosporin A, Surfonic NB-23®, and Peanut oil.

Another embodiment comprises cyclosporin A, Surfonic NB-25®, and Peanut oil.

Another embodiment comprises cyclosporin A, Surfonic NB-307®, and Peanut oil.

Another embodiment comprises cyclosporin A, Surfonic NB-31®, and Peanut oil.

Another embodiment comprises cyclosporin A, Surfonic NB-41®, and Peanut oil.

Another embodiment comprises cyclosporin A, Surfonic NB-43®, and Peanut oil.

Another embodiment comprises cyclosporin A, Surfonic OP-100®, and Peanut oil.

Another embodiment comprises cyclosporin A, Surfonic OP-120®, and Peanut oil.

Another embodiment comprises cyclosporin A, Surfonic OP-15®, and Peanut oil.

Another embodiment comprises cyclosporin A, Surfonic OP-35®, and Peanut oil.

Another embodiment comprises cyclosporin A, Surfonic OP-70®, and Peanut oil.

Another embodiment comprises cyclosporin A, Surfonic P1®, and Peanut oil.

Another embodiment comprises cyclosporin A, Surfonic P3®, and Peanut oil.

Another embodiment comprises cyclosporin A, Surfonic P5®, and Peanut oil.

Another embodiment comprises cyclosporin A, Surfonic P6®, and Peanut oil.

Another embodiment comprises cyclosporin A, Surfonic POA-17R2®, and Peanut oil.

Another embodiment comprises cyclosporin A, Surfonic POA-25R2®, and Peanut oil.

Another embodiment comprises cyclosporin A, Surfonic TDA-11®, and Peanut oil.

Another embodiment comprises cyclosporin A, Surfonic TDA-3B®, and Peanut oil.

Another embodiment comprises cyclosporin A, Surfonic TDA-6®, and Peanut oil.

Another embodiment comprises cyclosporin A, Surfonic TDA-6/88®, and Peanut oil.

Another embodiment comprises cyclosporin A, Surfonic TDA-8®, and Peanut oil.

Another embodiment comprises cyclosporin A, Surfonic TDA-8/90®, and Peanut oil.

Another embodiment comprises cyclosporin A, Surfonic TDA-9®, and Peanut oil.

Another embodiment comprises cyclosporin A, Surfonic TX-CCN®, and Peanut oil.

Another embodiment comprises cyclosporin A, Tagat CH 40®, and Peanut oil.

Another embodiment comprises cyclosporin A, Tagat CH 60®, and Peanut oil.

Another embodiment comprises cyclosporin A, Tagat L 2®, and Peanut oil.

Another embodiment comprises cyclosporin A, Tagat S®, and Peanut oil.

Another embodiment comprises cyclosporin A, Tagat S 2®, and Peanut oil.

Another embodiment comprises cyclosporin A, Tally 100 Plus®, and Peanut oil.

Another embodiment comprises cyclosporin A, T-Det A106®, and Peanut oil.

Another embodiment comprises cyclosporin A, T-Det A109®, and Peanut oil.

Another embodiment comprises cyclosporin A, T-Det A136®, and Peanut oil.

Another embodiment comprises cyclosporin A, T-Det A139®, and Peanut oil.

Another embodiment comprises cyclosporin A, T-Det A2412®, and Peanut oil.

Another embodiment comprises cyclosporin A, T-Det A243®, and Peanut oil.

Another embodiment comprises cyclosporin A, T-Det A247®, and Peanut oil.

Another embodiment comprises cyclosporin A, T-Det A249®, and Peanut oil.

Another embodiment comprises cyclosporin A, T-Det A467®, and Peanut oil.

Another embodiment comprises cyclosporin A, T-Det COE®, and Peanut oil.

Another embodiment comprises cyclosporin A, T-Det DD-10®, and Peanut oil.

Another embodiment comprises cyclosporin A, T-Det DD-5®, and Peanut oil.

Another embodiment comprises cyclosporin A, T-Det DD-7®, and Peanut oil.

Another embodiment comprises cyclosporin A, T-Det EPO-104®, and Peanut oil.

Another embodiment comprises cyclosporin A, T-Det EPO-61®, and Peanut oil.

Another embodiment comprises cyclosporin A, T-Det EPO-62®, and Peanut oil.

Another embodiment comprises cyclosporin A, T-Det EPO-64®, and Peanut oil.

Another embodiment comprises cyclosporin A, T-Det N-1.5®, and Peanut oil.

Another embodiment comprises cyclosporin A, T-Det N-10.5®, and Peanut oil.

Another embodiment comprises cyclosporin A, T-Det N-100®, and Peanut oil.

Another embodiment comprises cyclosporin A, T-Det N-1007®, and Peanut oil.

Another embodiment comprises cyclosporin A, T-Det N-12®, and Peanut oil.

Another embodiment comprises cyclosporin A, T-Det N-14®, and Peanut oil.

Another embodiment comprises cyclosporin A, T-Det N-20®, and Peanut oil.

Another embodiment comprises cyclosporin A, T-Det N-30®, and Peanut oil.

Another embodiment comprises cyclosporin A, T-Det N-307®, and Peanut oil.

Another embodiment comprises cyclosporin A, T-Det N-4®, and Peanut oil.

Another embodiment comprises cyclosporin A, T-Det N-40®, and Peanut oil.

Another embodiment comprises cyclosporin A, T-Det N-407®, and Peanut oil.

Another embodiment comprises cyclosporin A, T-Det N-50®, and Peanut oil.

Another embodiment comprises cyclosporin A, T-Det N-507®, and Peanut oil.

Another embodiment comprises cyclosporin A, T-Det N-6®, and Peanut oil.

Another embodiment comprises cyclosporin A, T-Det N-70®, and Peanut oil.

Another embodiment comprises cyclosporin A, T-Det N-8®, and Peanut oil.

Another embodiment comprises cyclosporin A, T-Det N-9.5®, and Peanut oil.

Another embodiment comprises cyclosporin A, T-Det O-12®, and Peanut oil.

Another embodiment comprises cyclosporin A, T-Det O-165®, and Peanut oil.

Another embodiment comprises cyclosporin A, T-Det O-307®, and Peanut oil.

Another embodiment comprises cyclosporin A, T-Det O-40®, and Peanut oil.

Another embodiment comprises cyclosporin A, T-Det O-407®, and Peanut oil.

Another embodiment comprises cyclosporin A, T-Det O-6®, and Peanut oil.

Another embodiment comprises cyclosporin A, T-Det O-8®, and Peanut oil.

Another embodiment comprises cyclosporin A, T-Det O-9®, and Peanut oil.

Another embodiment comprises cyclosporin A, Tegin®, and Peanut oil.

Another embodiment comprises cyclosporin A, Tergitol 15-S-12®, and Peanut oil.

Another embodiment comprises cyclosporin A, Tergitol 15-S-15®, and Peanut oil.

Another embodiment comprises cyclosporin A, Tergitol 15-S-20®, and Peanut oil.

Another embodiment comprises cyclosporin A, Tergitol 15-S-20 (80AQ)®, and Peanut oil.

Another embodiment comprises cyclosporin A, Tergitol 15-S-3®, and Peanut oil.

Another embodiment comprises cyclosporin A, Tergitol 15-S-30®, and Peanut oil.

Another embodiment comprises cyclosporin A, Tergitol 15-S-40®, and Peanut oil.

Another embodiment comprises cyclosporin A, Tergitol 15-S-40 (70AQ)®, and Peanut oil.

Another embodiment comprises cyclosporin A, Tergitol 15-S-5®, and Peanut oil.

Another embodiment comprises cyclosporin A, Tergitol 15-S-7®, and Peanut oil.

Another embodiment comprises cyclosporin A, Tergitol 15-S-9®, and Peanut oil.

Another embodiment comprises cyclosporin A, Tergitol L-101®, and Peanut oil.

Another embodiment comprises cyclosporin A, Tergitol L-61®, and Peanut oil.

Another embodiment comprises cyclosporin A, Tergitol L-62®, and Peanut oil.

Another embodiment comprises cyclosporin A, Tergitol L-64®, and Peanut oil.

Another embodiment comprises cyclosporin A, Tergitol L-81®, and Peanut oil.

Another embodiment comprises cyclosporin A, Tergitol NP-10®, and Peanut oil.

Another embodiment comprises cyclosporin A, Tergitol NP-11®, and Peanut oil.

Another embodiment comprises cyclosporin A, Tergitol NP-12®, and Peanut oil.

Another embodiment comprises cyclosporin A, Tergitol NP-13®, and Peanut oil.

Another embodiment comprises cyclosporin A, Tergitol NP-15®, and Peanut oil.

Another embodiment comprises cyclosporin A, Tergitol NP-30®, and Peanut oil.

Another embodiment comprises cyclosporin A, Tergitol NP-30(70 AQ)®, and Peanut oil.

Another embodiment comprises cyclosporin A, Tergitol NP-4®, and Peanut oil.

Another embodiment comprises cyclosporin A, Tergitol NP-40®, and Peanut oil.

Another embodiment comprises cyclosporin A, Tergitol NP-40 (70 AQ)®, and Peanut oil.

Another embodiment comprises cyclosporin A, Tergitol NP-50 (70 AQ)®, and Peanut oil.

Another embodiment comprises cyclosporin A, Tergitol NP-55 (70 AQ)®, and Peanut oil.

Another embodiment comprises cyclosporin A, Tergitol NP-6®, and Peanut oil.

Another embodiment comprises cyclosporin A, Tergitol NP-7®, and Peanut oil.

Another embodiment comprises cyclosporin A, Tergitol NP-70 (70 AQ)®, and Peanut oil.

Another embodiment comprises cyclosporin A, Tergitol NP-8®, and Peanut oil.

Another embodiment comprises cyclosporin A, Tergitol NP-9®, and Peanut oil.

Another embodiment comprises cyclosporin A, Tergitol NP-9.5®, and Peanut oil.

Another embodiment comprises cyclosporin A, Tergitol TMN-10®, and Peanut oil.

Another embodiment comprises cyclosporin A, Tergitol TMN-3®, and Peanut oil.

Another embodiment comprises cyclosporin A, Tergitol TMN-6®, and Peanut oil.

Another embodiment comprises cyclosporin A, Tergitol XD®, and Peanut oil.

Another embodiment comprises cyclosporin A, Tergitol XH®, and Peanut oil.

Another embodiment comprises cyclosporin A, Tergitol XJ®, and Peanut oil.

Another embodiment comprises cyclosporin A, Tetronic 1107®, and Peanut oil.

Another embodiment comprises cyclosporin A, Tetronic 1307®, and Peanut oil.

Another embodiment comprises cyclosporin A, Tetronic 150R1®, and Peanut oil.

Another embodiment comprises cyclosporin A, Tetronic 304®, and Peanut oil.

Another embodiment comprises cyclosporin A, Tetronic 701®, and Peanut oil.

Another embodiment comprises cyclosporin A, Tetronic 901®, and Peanut oil.

Another embodiment comprises cyclosporin A, Tetronic 904®, and Peanut oil.

Another embodiment comprises cyclosporin A, Tetronic 908®, and Peanut oil.

Another embodiment comprises cyclosporin A, Tetronic 90R4®, and Peanut oil.

Another embodiment comprises cyclosporin A, Tomadol 1-3®, and Peanut oil.

Another embodiment comprises cyclosporin A, Tomadol 1-5®, and Peanut oil.

Another embodiment comprises cyclosporin A, Tomadol 1-7®, and Peanut oil.

Another embodiment comprises cyclosporin A, Tomadol 23-1®, and Peanut oil.

Another embodiment comprises cyclosporin A, Tomadol 23-3®, and Peanut oil.

Another embodiment comprises cyclosporin A, Tomadol 23-5®, and Peanut oil.

Another embodiment comprises cyclosporin A, Tomadol 23-6.5®, and Peanut oil.

Another embodiment comprises cyclosporin A, Tomadol 25-12®, and Peanut oil.

Another embodiment comprises cyclosporin A, Tomadol 25-3®, and Peanut oil.

Another embodiment comprises cyclosporin A, Tomadol 25-7®, and Peanut oil.

Another embodiment comprises cyclosporin A, Tomadol 25-9®, and Peanut oil.

Another embodiment comprises cyclosporin A, Tomadol 45-13®, and Peanut oil.

Another embodiment comprises cyclosporin A, Tomadol 45-2.5®, and Peanut oil.

Another embodiment comprises cyclosporin A, Tomadol 45-7®, and Peanut oil.

Another embodiment comprises cyclosporin A, Tomadol 91-2.5®, and Peanut oil.

Another embodiment comprises cyclosporin A, Tomadol 91-6®, and Peanut oil.

Another embodiment comprises cyclosporin A, Tomadol 91-8®, and Peanut oil.

Another embodiment comprises cyclosporin A, Triton X-100®, and Peanut oil.

Another embodiment comprises cyclosporin A, Triton X-102®, and Peanut oil.

Another embodiment comprises cyclosporin A, Triton X-114®, and Peanut oil.

Another embodiment comprises cyclosporin A, Triton X-15®, and Peanut oil.

Another embodiment comprises cyclosporin A, Triton X-165 (70%)®, and Peanut oil.

Another embodiment comprises cyclosporin A, Triton X-207®, and Peanut oil.

Another embodiment comprises cyclosporin A, Triton X-305 (70%)®, and Peanut oil.

Another embodiment comprises cyclosporin A, Triton X-35®, and Peanut oil.

Another embodiment comprises cyclosporin A, Triton X-405 (70%)®, and Peanut oil.

Another embodiment comprises cyclosporin A, Triton X-45®, and Peanut oil.

Another embodiment comprises cyclosporin A, Triton X-705 (70%)®, and Peanut oil.

Another embodiment comprises cyclosporin A, Volpo S-10®, and Peanut oil.

Another embodiment comprises cyclosporin A, Volpo S-2®, and Peanut oil.

Another embodiment comprises cyclosporin A, Volpo S-20®, and Peanut oil.

Another embodiment comprises cyclosporin A, Volpo-10®, and Peanut oil.

Another embodiment comprises cyclosporin A, Volpo-20®, and Peanut oil.

Another embodiment comprises cyclosporin A, Volpo-3®, and Peanut oil.

Another embodiment comprises cyclosporin A, Volpo-5®, and Peanut oil.

Another embodiment comprises cyclosporin A, Abex 2515®, and Peppermint oil.

Another embodiment comprises cyclosporin A, Abex 2525/40®, and Peppermint oil.

Another embodiment comprises cyclosporin A, Abex 2535®, and Peppermint oil.

Another embodiment comprises cyclosporin A, Abex 2545®, and Peppermint oil.

Another embodiment comprises cyclosporin A, Acconon E®, and Peppermint oil.

Another embodiment comprises cyclosporin A, Alfonic 1216CO-9®, and Peppermint oil.

Another embodiment comprises cyclosporin A, Alfonic 1412-3®, and Peppermint oil.

Another embodiment comprises cyclosporin A, Alfonic TDA-12®, and Peppermint oil.

Another embodiment comprises cyclosporin A, Alfonic TDA-3®, and Peppermint oil.

Another embodiment comprises cyclosporin A, Alfonic TDA-4®, and Peppermint oil.

Another embodiment comprises cyclosporin A, Alfonic TDA-6®, and Peppermint oil.

Another embodiment comprises cyclosporin A, Alfonic TDA-7®, and Peppermint oil.

Another embodiment comprises cyclosporin A, Alfonic TDA-8®, and Peppermint oil.

Another embodiment comprises cyclosporin A, Alfonic TDA-9®, and Peppermint oil.

Another embodiment comprises cyclosporin A, Alkamuls 400-DO®, and Peppermint oil.

Another embodiment comprises cyclosporin A, Alkamuls 400-MO/E®, and Peppermint oil.

Another embodiment comprises cyclosporin A, Ameroxol OE-10®, and Peppermint oil.

Another embodiment comprises cyclosporin A, Ameroxol OE-2®, and Peppermint oil.

Another embodiment comprises cyclosporin A, Ameroxol OE-20®, and Peppermint oil.

Another embodiment comprises cyclosporin A, Armix 176®, and Peppermint oil.

Another embodiment comprises cyclosporin A, Armix 180-C®, and Peppermint oil.

Another embodiment comprises cyclosporin A, Armix 183®, and Peppermint oil.

Another embodiment comprises cyclosporin A, Brij 35®, and Peppermint oil.

Another embodiment comprises cyclosporin A, Brij 35Liquid®, and Peppermint oil.

Another embodiment comprises cyclosporin A, Brij 35SP®, and Peppermint oil.

Another embodiment comprises cyclosporin A, Canasol MJ 2109®, and Peppermint oil.

Another embodiment comprises cyclosporin A, Canasol MJ 52®, and Peppermint oil.

Another embodiment comprises cyclosporin A, Canasol R 3600®, and Peppermint oil.

Another embodiment comprises cyclosporin A, Canasol R 3603®, and Peppermint oil.

Another embodiment comprises cyclosporin A, Canasol R 4000 H®, and Peppermint oil.

Another embodiment comprises cyclosporin A, Cedepal CA-210®, and Peppermint oil.

Another embodiment comprises cyclosporin A, Cedepal CA-520®, and Peppermint oil.

Another embodiment comprises cyclosporin A, Cedepal CA-630®, and Peppermint oil.

Another embodiment comprises cyclosporin A, Cedepal CA-720®, and Peppermint oil.

Another embodiment comprises cyclosporin A, Cedepal CA-890®, and Peppermint oil.

Another embodiment comprises cyclosporin A, Cedepal CA-897®, and Peppermint oil.

Another embodiment comprises cyclosporin A, Cedepal CO-210®, and Peppermint oil.

Another embodiment comprises cyclosporin A, Cedepal CO-430®, and Peppermint oil.

Another embodiment comprises cyclosporin A, Cedepal CO-530®, and Peppermint oil.

Another embodiment comprises cyclosporin A, Cedepal CO-630®, and Peppermint oil.

Another embodiment comprises cyclosporin A, Cedepal CO-710®, and Peppermint oil.

Another embodiment comprises cyclosporin A, Cedepal CO-730®, and Peppermint oil.

Another embodiment comprises cyclosporin A, Cedepal CO-880®, and Peppermint oil.

Another embodiment comprises cyclosporin A, Cedepal CO-887®, and Peppermint oil.

Another embodiment comprises cyclosporin A, Cedepal CO-890®, and Peppermint oil.

Another embodiment comprises cyclosporin A, Cedepal CO-897®, and Peppermint oil.

Another embodiment comprises cyclosporin A, Cedepal CO-970®, and Peppermint oil.

Another embodiment comprises cyclosporin A, Cedepal CO-977®, and Peppermint oil.

Another embodiment comprises cyclosporin A, Cedepal CO-990®, and Peppermint oil.

Another embodiment comprises cyclosporin A, Cedepal CO-997®, and Peppermint oil.

Another embodiment comprises cyclosporin A, Chemal BP 261®, and Peppermint oil.

Another embodiment comprises cyclosporin A, Chemal BP 262®, and Peppermint oil.

Another embodiment comprises cyclosporin A, Chemal BP 3172®, and Peppermint oil.

Another embodiment comprises cyclosporin A, Chemal BP 3174®, and Peppermint oil.

Another embodiment comprises cyclosporin A, Chemal DA-4®, and Peppermint oil.

Another embodiment comprises cyclosporin A, Chemal DA-6®, and Peppermint oil.

Another embodiment comprises cyclosporin A, Chemal DA-9®, and Peppermint oil.

Another embodiment comprises cyclosporin A, Chemal LA-23®, and Peppermint oil.

Another embodiment comprises cyclosporin A, Chemal LA-4®, and Peppermint oil.

Another embodiment comprises cyclosporin A, Chemal LA-9®, and Peppermint oil.

Another embodiment comprises cyclosporin A, Chemal OA-20G®, and Peppermint oil.

Another embodiment comprises cyclosporin A, Chemal OA-5®, and Peppermint oil.

Another embodiment comprises cyclosporin A, Chemal OA-9®, and Peppermint oil.

Another embodiment comprises cyclosporin A, Chemal TDA-12®, and Peppermint oil.

Another embodiment comprises cyclosporin A, Chemal TDA-15®, and Peppermint oil.

Another embodiment comprises cyclosporin A, Chemal TDA-3®, and Peppermint oil.

Another embodiment comprises cyclosporin A, Chemal TDA-6®, and Peppermint oil.

Another embodiment comprises cyclosporin A, Chemal TDA-9®, and Peppermint oil.

Another embodiment comprises cyclosporin A, Chemax CO-16®, and Peppermint oil.

Another embodiment comprises cyclosporin A, Chemax CO-200/50®, and Peppermint oil.

Another embodiment comprises cyclosporin A, Chemax CO-25®, and Peppermint oil.

Another embodiment comprises cyclosporin A, Chemax CO-30®, and Peppermint oil.

Another embodiment comprises cyclosporin A, Chemax CO-36®, and Peppermint oil.

Another embodiment comprises cyclosporin A, Chemax CO-40®, and Peppermint oil.

Another embodiment comprises cyclosporin A, Chemax CO-5®, and Peppermint oil.

Another embodiment comprises cyclosporin A, Chemax CO-80®, and Peppermint oil.

Another embodiment comprises cyclosporin A, Chemax DNP-150/50®, and Peppermint oil.

Another embodiment comprises cyclosporin A, Chemax DNP-18®, and Peppermint oil.

Another embodiment comprises cyclosporin A, Chemax DNP-8®, and Peppermint oil.

Another embodiment comprises cyclosporin A, Chemax E 200 ML®, and Peppermint oil.

Another embodiment comprises cyclosporin A, Chemax E 200 MO®, and Peppermint oil.

Another embodiment comprises cyclosporin A, Chemax E 400 ML®, and Peppermint oil.

Another embodiment comprises cyclosporin A, Chemax E 400 MO®, and Peppermint oil.

Another embodiment comprises cyclosporin A, Chemax E 600 ML®, and Peppermint oil.

Another embodiment comprises cyclosporin A, Chemax E 600 MO®, and Peppermint oil.

Another embodiment comprises cyclosporin A, Chemax El 1000 MO®, and Peppermint oil.

Another embodiment comprises cyclosporin A, Chemax El 1000 MS®, and Peppermint oil.

Another embodiment comprises cyclosporin A, Chemax E200 MS®, and Peppermint oil.

Another embodiment comprises cyclosporin A, Chemax E400 MS®, and Peppermint oil.

Another embodiment comprises cyclosporin A, Chemax E600 MS®, and Peppermint oil.

Another embodiment comprises cyclosporin A, Chemax HCO-16®, and Peppermint oil.

Another embodiment comprises cyclosporin A, Chemax HCO-200/50®, and Peppermint oil.

Another embodiment comprises cyclosporin A, Chemax HCO-25®, and Peppermint oil.

Another embodiment comprises cyclosporin A, Chemax HCO-5®, and Peppermint oil.

Another embodiment comprises cyclosporin A, Chemax TO-16®, and Peppermint oil.

Another embodiment comprises cyclosporin A, Chemax TO-8®, and Peppermint oil.

Another embodiment comprises cyclosporin A, Colalube 3409®, and Peppermint oil.

Another embodiment comprises cyclosporin A, Colalube 3411®, and Peppermint oil.

Another embodiment comprises cyclosporin A, Colalube 3413®, and Peppermint oil.

Another embodiment comprises cyclosporin A, Colalube 3414®, and Peppermint oil.

Another embodiment comprises cyclosporin A, Colalube 3415®, and Peppermint oil.

Another embodiment comprises cyclosporin A, Colalube 3416®, and Peppermint oil.

Another embodiment comprises cyclosporin A, Colalube 3417®, and Peppermint oil.

Another embodiment comprises cyclosporin A, Delonic NPE-100®, and Peppermint oil.

Another embodiment comprises cyclosporin A, Delonic NPE-30®, and Peppermint oil.

Another embodiment comprises cyclosporin A, Delonic NPE-40®, and Peppermint oil.

Another embodiment comprises cyclosporin A, Delonic OPE-10®, and Peppermint oil.

Another embodiment comprises cyclosporin A, Delonic OPE-12®, and Peppermint oil.

Another embodiment comprises cyclosporin A, Delonic OPE-30®, and Peppermint oil.

Another embodiment comprises cyclosporin A, Delonic OPE-40®, and Peppermint oil.

Another embodiment comprises cyclosporin A, Delonic OPE-5®, and Peppermint oil.

Another embodiment comprises cyclosporin A, Delonic OPE-7.5®, and Peppermint oil.

Another embodiment comprises cyclosporin A, DePeg 16-CO®, and Peppermint oil.

Another embodiment comprises cyclosporin A, DePeg 25-CO®, and Peppermint oil.

Another embodiment comprises cyclosporin A, DePeg 30-CO®, and Peppermint oil.

Another embodiment comprises cyclosporin A, DePeg 40-CO®, and Peppermint oil.

Another embodiment comprises cyclosporin A, DePeg 5-CO®, and Peppermint oil.

Another embodiment comprises cyclosporin A, DePeg 80-CO®, and Peppermint oil.

Another embodiment comprises cyclosporin A, DeSonic S-100®, and Peppermint oil.

Another embodiment comprises cyclosporin A, DeSonic S-114®, and Peppermint oil.

Another embodiment comprises cyclosporin A, DeSonic S-405®, and Peppermint oil.

Another embodiment comprises cyclosporin A, DeSonic S-45®, and Peppermint oil.

Another embodiment comprises cyclosporin A, DeSonic TDA-9®, and Peppermint oil.

Another embodiment comprises cyclosporin A, DeThox Acid O-14®, and Peppermint oil.

Another embodiment comprises cyclosporin A, DeThox Acid O-16®, and Peppermint oil.

Another embodiment comprises cyclosporin A, DeThox Acid O-9®, and Peppermint oil.

Another embodiment comprises cyclosporin A, DeThox Acid TO-14®, and Peppermint oil.

Another embodiment comprises cyclosporin A, DeThox Acid TO-16.5®, and Peppermint oil.

Another embodiment comprises cyclosporin A, DeThox Acid TO-8.5®, and Peppermint oil.

Another embodiment comprises cyclosporin A, Dur-Em 114®, and Peppermint oil.

Another embodiment comprises cyclosporin A, Dur-Em 117®, and Peppermint oil.

Another embodiment comprises cyclosporin A, Dur-Em 204®, and Peppermint oil.

Another embodiment comprises cyclosporin A, Dur-Em 207®, and Peppermint oil.

Another embodiment comprises cyclosporin A, Dur-Em 207E®, and Peppermint oil.

Another embodiment comprises cyclosporin A, Emerest 2704®, and Peppermint oil.

Another embodiment comprises cyclosporin A, Emerest 2712®, and Peppermint oil.

Another embodiment comprises cyclosporin A, Emerest 2715®, and Peppermint oil.

Another embodiment comprises cyclosporin A, Ethal EH-2®, and Peppermint oil.

Another embodiment comprises cyclosporin A, Ethal EH-5®, and Peppermint oil.

Another embodiment comprises cyclosporin A, Ethal OA-23®, and Peppermint oil.

Another embodiment comprises cyclosporin A, Ethosperse CA-20®, and Peppermint oil.

Another embodiment comprises cyclosporin A, Ethosperse LA-12®, and Peppermint oil.

Another embodiment comprises cyclosporin A, Ethosperse LA-23®, and Peppermint oil.

Another embodiment comprises cyclosporin A, Ethosperse LA-4®, and Peppermint oil.

Another embodiment comprises cyclosporin A, Ethox 3095®, and Peppermint oil.

Another embodiment comprises cyclosporin A, Ethox CO-16®, and Peppermint oil.

Another embodiment comprises cyclosporin A, Ethox CO-200®, and Peppermint oil.

Another embodiment comprises cyclosporin A, Ethox CO-25®, and Peppermint oil.

Another embodiment comprises cyclosporin A, Ethox CO-30®, and Peppermint oil.

Another embodiment comprises cyclosporin A, Ethox CO-36®, and Peppermint oil.

Another embodiment comprises cyclosporin A, Ethox CO-40®, and Peppermint oil.

Another embodiment comprises cyclosporin A, Ethox CO-5®, and Peppermint oil.

Another embodiment comprises cyclosporin A, Ethox DO-14®, and Peppermint oil.

Another embodiment comprises cyclosporin A, Ethox DO-9®, and Peppermint oil.

Another embodiment comprises cyclosporin A, Ethox MA-8®, and Peppermint oil.

Another embodiment comprises cyclosporin A, Ethox MI-14®, and Peppermint oil.

Another embodiment comprises cyclosporin A, Ethox ML-14®, and Peppermint oil.

Another embodiment comprises cyclosporin A, Ethox ML-5®, and Peppermint oil.

Another embodiment comprises cyclosporin A, Ethox MO-14®, and Peppermint oil.

Another embodiment comprises cyclosporin A, Ethox MO-9®, and Peppermint oil.

Another embodiment comprises cyclosporin A, Ethox MS-14®, and Peppermint oil.

Another embodiment comprises cyclosporin A, Ethox MS-23®, and Peppermint oil.

Another embodiment comprises cyclosporin A, Ethox MS-40®, and Peppermint oil.

Another embodiment comprises cyclosporin A, Ethox MS-8®, and Peppermint oil.

Another embodiment comprises cyclosporin A, Ethox TO-16®, and Peppermint oil.

Another embodiment comprises cyclosporin A, Ethox TO-8®, and Peppermint oil.

Another embodiment comprises cyclosporin A, Ethylan SN-120®, and Peppermint oil.

Another embodiment comprises cyclosporin A, Ethylan SN-70®, and Peppermint oil.

Another embodiment comprises cyclosporin A, Ethylan SN-90®, and Peppermint oil.

Another embodiment comprises cyclosporin A, Flexricin 13®, and Peppermint oil.

Another embodiment comprises cyclosporin A, Flexricin 15®, and Peppermint oil.

Another embodiment comprises cyclosporin A, Genapol 1261®, and Peppermint oil.

Another embodiment comprises cyclosporin A, Generol 122 E250®, and Peppermint oil.

Another embodiment comprises cyclosporin A, Glicepol 160®, and Peppermint oil.

Another embodiment comprises cyclosporin A, Glicepol 560®, and Peppermint oil.

Another embodiment comprises cyclosporin A, Glicepol GMS 20®, and Peppermint oil.

Another embodiment comprises cyclosporin A, Hetoxide BN-13®, and Peppermint oil.

Another embodiment comprises cyclosporin A, Hetoxide DNP-4®, and Peppermint oil.

Another embodiment comprises cyclosporin A, Hetoxide DNP-9.6®, and Peppermint oil.

Another embodiment comprises cyclosporin A, Hetoxide G-26®, and Peppermint oil.

Another embodiment comprises cyclosporin A, Hetoxide G-7®, and Peppermint oil.

Another embodiment comprises cyclosporin A, Hetoxide GT-20®, and Peppermint oil.

Another embodiment comprises cyclosporin A, Hetoxide HC-40®, and Peppermint oil.

Another embodiment comprises cyclosporin A, Hetoxol C-24®, and Peppermint oil.

Another embodiment comprises cyclosporin A, Hetoxol CA-10®, and Peppermint oil.

Another embodiment comprises cyclosporin A, Hetoxol CA-2®, and Peppermint oil.

Another embodiment comprises cyclosporin A, Hetoxol CA-20®, and Peppermint oil.

Another embodiment comprises cyclosporin A, Hetoxol CAWS®, and Peppermint oil.

Another embodiment comprises cyclosporin A, Hetoxol CD-3®, and Peppermint oil.

Another embodiment comprises cyclosporin A, Hetoxol CD-4®, and Peppermint oil.

Another embodiment comprises cyclosporin A, Hetoxol CS-15®, and Peppermint oil.

Another embodiment comprises cyclosporin A, Hetoxol CS-20®, and Peppermint oil.

Another embodiment comprises cyclosporin A, Hetoxol CS-20D®, and Peppermint oil.

Another embodiment comprises cyclosporin A, Hetoxol CS-25®, and Peppermint oil.

Another embodiment comprises cyclosporin A, Hetoxol CS-50®, and Peppermint oil.

Another embodiment comprises cyclosporin A, Hetoxol CS-9®, and Peppermint oil.

Another embodiment comprises cyclosporin A, Hetoxol CSA-15®, and Peppermint oil.

Another embodiment comprises cyclosporin A, Hetoxol D®, and Peppermint oil.

Another embodiment comprises cyclosporin A, Hetoxol G®, and Peppermint oil.

Another embodiment comprises cyclosporin A, Hetoxol J®, and Peppermint oil.

Another embodiment comprises cyclosporin A, Hetoxol L®, and Peppermint oil.

Another embodiment comprises cyclosporin A, Hetoxol L-23®, and Peppermint oil.

Another embodiment comprises cyclosporin A, Hetoxol L-4®, and Peppermint oil.

Another embodiment comprises cyclosporin A, Hetoxol L-9®, and Peppermint oil.

Another embodiment comprises cyclosporin A, Hetoxol LS-9®, and Peppermint oil.

Another embodiment comprises cyclosporin A, Hetoxol M-3®, and Peppermint oil.

Another embodiment comprises cyclosporin A, Hetoxol OA-10 Special®, and Peppermint oil.

Another embodiment comprises cyclosporin A, Hetoxol OA-20 Special®, and Peppermint oil.

Another embodiment comprises cyclosporin A, Hetoxol OA-3 Special®, and Peppermint oil.

Another embodiment comprises cyclosporin A, Hetoxol OA-5 Special®, and Peppermint oil.

Another embodiment comprises cyclosporin A, Hetoxol OL-10®, and Peppermint oil.

Another embodiment comprises cyclosporin A, Hetoxol OL-2®, and Peppermint oil.

Another embodiment comprises cyclosporin A, Hetoxol OL-23®, and Peppermint oil.

Another embodiment comprises cyclosporin A, Hetoxol OL-4®, and Peppermint oil.

Another embodiment comprises cyclosporin A, Hetoxol OL-40®, and Peppermint oil.

Another embodiment comprises cyclosporin A, Hetoxol OL-5®, and Peppermint oil.

Another embodiment comprises cyclosporin A, Hetoxol PLA®, and Peppermint oil.

Another embodiment comprises cyclosporin A, Hetoxol SP-15®, and Peppermint oil.

Another embodiment comprises cyclosporin A, Hetoxol STA-10®, and Peppermint oil.

Another embodiment comprises cyclosporin A, Hetoxol STA-2®, and Peppermint oil.

Another embodiment comprises cyclosporin A, Hetoxol STA-30®, and Peppermint oil.

Another embodiment comprises cyclosporin A, Hetoxol TD-12®, and Peppermint oil.

Another embodiment comprises cyclosporin A, Hetoxol TD-18®, and Peppermint oil.

Another embodiment comprises cyclosporin A, Hetoxol TD-3®, and Peppermint oil.

Another embodiment comprises cyclosporin A, Hetoxol TD-6®, and Peppermint oil.

Another embodiment comprises cyclosporin A, Hetoxol TD-9®, and Peppermint oil.

Another embodiment comprises cyclosporin A, Hetoxol TDEP-15®, and Peppermint oil.

Another embodiment comprises cyclosporin A, Hetoxol TDEP-63®, and Peppermint oil.

Another embodiment comprises cyclosporin A, Iconol 24-12®, and Peppermint oil.

Another embodiment comprises cyclosporin A, Iconol 24-23®, and Peppermint oil.

Another embodiment comprises cyclosporin A, Iconol 24-4®, and Peppermint oil.

Another embodiment comprises cyclosporin A, Iconol DA-4®, and Peppermint oil.

Another embodiment comprises cyclosporin A, Iconol DA-6®, and Peppermint oil.

Another embodiment comprises cyclosporin A, Iconol TDA-10®, and Peppermint oil.

Another embodiment comprises cyclosporin A, Iconol TDA-3®, and Peppermint oil.

Another embodiment comprises cyclosporin A, Iconol TDA-6®, and Peppermint oil.

Another embodiment comprises cyclosporin A, Iconol TDA-8-90%®, and Peppermint oil.

Another embodiment comprises cyclosporin A, Iconol TDA-9®, and Peppermint oil.

Another embodiment comprises cyclosporin A, Igepal CA-210®, and Peppermint oil.

Another embodiment comprises cyclosporin A, Igepal CA-407®, and Peppermint oil.

Another embodiment comprises cyclosporin A, Igepal CA-420®, and Peppermint oil.

Another embodiment comprises cyclosporin A, Igepal CA-520®, and Peppermint oil.

Another embodiment comprises cyclosporin A, Igepal CA-620®, and Peppermint oil.

Another embodiment comprises cyclosporin A, Igepal CA-630®, and Peppermint oil.

Another embodiment comprises cyclosporin A, Igepal CA-720®, and Peppermint oil.

Another embodiment comprises cyclosporin A, Igepal CA-877®, and Peppermint oil.

Another embodiment comprises cyclosporin A, Igepal CA-887®, and Peppermint oil.

Another embodiment comprises cyclosporin A, Igepal CA-897®, and Peppermint oil.

Another embodiment comprises cyclosporin A, Igepal CO-210®, and Peppermint oil.

Another embodiment comprises cyclosporin A, Igepal CO-430®, and Peppermint oil.

Another embodiment comprises cyclosporin A, Igepal CO-520®, and Peppermint oil.

Another embodiment comprises cyclosporin A, Igepal CO-530®, and Peppermint oil.

Another embodiment comprises cyclosporin A, Igepal CO-610®, and Peppermint oil.

Another embodiment comprises cyclosporin A, Igepal CO-630®, and Peppermint oil.

Another embodiment comprises cyclosporin A, Igepal CO-630 Special®, and Peppermint oil.

Another embodiment comprises cyclosporin A, Igepal CO-660®, and Peppermint oil.

Another embodiment comprises cyclosporin A, Igepal CO-710®, and Peppermint oil.

Another embodiment comprises cyclosporin A, Igepal CO-720®, and Peppermint oil.

Another embodiment comprises cyclosporin A, Igepal CO-730®, and Peppermint oil.

Another embodiment comprises cyclosporin A, Igepal CO-738®, and Peppermint oil.

Another embodiment comprises cyclosporin A, Igepal CO-850®, and Peppermint oil.

Another embodiment comprises cyclosporin A, Igepal CO-880®, and Peppermint oil.

Another embodiment comprises cyclosporin A, Igepal CO-887®, and Peppermint oil.

Another embodiment comprises cyclosporin A, Igepal CO-890®, and Peppermint oil.

Another embodiment comprises cyclosporin A, Igepal CO-897®, and Peppermint oil.

Another embodiment comprises cyclosporin A, Igepal CO-970®, and Peppermint oil.

Another embodiment comprises cyclosporin A, Igepal CO-977®, and Peppermint oil.

Another embodiment comprises cyclosporin A, Igepal CO-987®, and Peppermint oil.

Another embodiment comprises cyclosporin A, Igepal CO-990 FLK®, and Peppermint oil.

Another embodiment comprises cyclosporin A, Igepal CO-997®, and Peppermint oil.

Another embodiment comprises cyclosporin A, Igepal DM-430®, and Peppermint oil.

Another embodiment comprises cyclosporin A, Igepal DM-530®, and Peppermint oil.

Another embodiment comprises cyclosporin A, Igepal DM-710®, and Peppermint oil.

Another embodiment comprises cyclosporin A, Igepal DM-730®, and Peppermint oil.

Another embodiment comprises cyclosporin A, Igepal DM-880®, and Peppermint oil.

Another embodiment comprises cyclosporin A, Igepal DM-970 FLK®, and Peppermint oil.

Another embodiment comprises cyclosporin A, Igepal OD-410®, and Peppermint oil.

Another embodiment comprises cyclosporin A, Igepal RC-520®, and Peppermint oil.

Another embodiment comprises cyclosporin A, Igepal RC-620®, and Peppermint oil.

Another embodiment comprises cyclosporin A, Igepal RC-630®, and Peppermint oil.

Another embodiment comprises cyclosporin A, Lexemul CS-20®, and Peppermint oil.

Another embodiment comprises cyclosporin A, Lipo GMS 450®, and Peppermint oil.

Another embodiment comprises cyclosporin A, Lipo Polyglycol 1000®, and Peppermint oil.

Another embodiment comprises cyclosporin A, Lipo Polyglycol 200®, and Peppermint oil.

Another embodiment comprises cyclosporin A, Lipo Polyglycol 300®, and Peppermint oil.

Another embodiment comprises cyclosporin A, Lipo Polyglycol 3350®, and Peppermint oil.

Another embodiment comprises cyclosporin A, Lipo Polyglycol 400®, and Peppermint oil.

Another embodiment comprises cyclosporin A, Lipo Polyglycol 600®, and Peppermint oil.

Another embodiment comprises cyclosporin A, Lipocol C-10®, and Peppermint oil.

Another embodiment comprises cyclosporin A, Lipocol C-2®, and Peppermint oil.

Another embodiment comprises cyclosporin A, Lipocol C-20®, and Peppermint oil.

Another embodiment comprises cyclosporin A, Lipocol L-12®, and Peppermint oil.

Another embodiment comprises cyclosporin A, Lipocol L-23®, and Peppermint oil.

Another embodiment comprises cyclosporin A, Lipocol L-4®, and Peppermint oil.

Another embodiment comprises cyclosporin A, Lipocol O-10®, and Peppermint oil.

Another embodiment comprises cyclosporin A, Lipocol O-2®, and Peppermint oil.

Another embodiment comprises cyclosporin A, Lipocol O-20®and Peppermint oil.

Another embodiment comprises cyclosporin A, Lipocol O-5®, and Peppermint oil.

Another embodiment comprises cyclosporin A, Lipocol S-10®, and Peppermint oil.

Another embodiment comprises cyclosporin A, Lipocol S-2®, and Peppermint oil.

Another embodiment comprises cyclosporin A, Lipocol S-20®, and Peppermint oil.

Another embodiment comprises cyclosporin A, Lipocol SC-15®, and Peppermint oil.

Another embodiment comprises cyclosporin A, Lipocol SC-20®, and Peppermint oil.

Another embodiment comprises cyclosporin A, Lipocol SC-4®, and Peppermint oil.

Another embodiment comprises cyclosporin A, Lipocol TD-12®, and Peppermint oil.

Another embodiment comprises cyclosporin A, Lumulse 40-O K®, and Peppermint oil.

Another embodiment comprises cyclosporin A, Lumulse 42-O K®, and Peppermint oil.

Another embodiment comprises cyclosporin A, Lumulse GMT-40®, and Peppermint oil.

Another embodiment comprises cyclosporin A, Lumulse PEG 1450®, and Peppermint oil.

Another embodiment comprises cyclosporin A, Lumulse PEG 1450 NF®, and Peppermint oil.

Another embodiment comprises cyclosporin A, Lumulse PEG 8000®, and Peppermint oil.

Another embodiment comprises cyclosporin A, Lumulse PGO®, and Peppermint oil.

Another embodiment comprises cyclosporin A, Lumulse POE (2) Oleyl Amine®, and Peppermint oil.

Another embodiment comprises cyclosporin A, Magrabar PGO-104®, and Peppermint oil.

Another embodiment comprises cyclosporin A, Mapeg 200 ML®, and Peppermint oil.

Another embodiment comprises cyclosporin A, Mapeg 400 MO®, and Peppermint oil.

Another embodiment comprises cyclosporin A, Mapeg 400 MOT®, and Peppermint oil.

Another embodiment comprises cyclosporin A, Mapeg 400DO®, and Peppermint oil.

Another embodiment comprises cyclosporin A, Mapeg 400DOT®, and Peppermint oil.

Another embodiment comprises cyclosporin A, Mapeg 600DOT®, and Peppermint oil.

Another embodiment comprises cyclosporin A, Marlox B 24/80®, and Peppermint oil.

Another embodiment comprises cyclosporin A, Marlox FK 86®, and Peppermint oil.

Another embodiment comprises cyclosporin A, Marlox MO 154®, and Peppermint oil.

Another embodiment comprises cyclosporin A, Mazox CDA CG®, and Peppermint oil.

Another embodiment comprises cyclosporin A, Monoglycerides Glyceryl Monestearate®, and Peppermint oil.

Another embodiment comprises cyclosporin A, Myrj 45®, and Peppermint oil.

Another embodiment comprises cyclosporin A, Myrj 52®, and Peppermint oil.

Another embodiment comprises cyclosporin A, Myrj 52S®, and Peppermint oil.

Another embodiment comprises cyclosporin A, Myrj 53®, and Peppermint oil.

Another embodiment comprises cyclosporin A, Myrj 59®, and Peppermint oil.

Another embodiment comprises cyclosporin A, Norfox NP-12®, and Peppermint oil.

Another embodiment comprises cyclosporin A, Norfox NP-4®, and Peppermint oil.

Another embodiment comprises cyclosporin A, Nodox NP-6®, and Peppermint oil.

Another embodiment comprises cyclosporin A, Norfox NP-878®, and Peppermint oil.

Another embodiment comprises cyclosporin A, Nordox NP-9®, and Peppermint oil.

Another embodiment comprises cyclosporin A, Nordox NP-977®, and Peppermint oil.

Another embodiment comprises cyclosporin A, Norfox OP-100®, and Peppermint oil.

Another embodiment comprises cyclosporin A, Nordox OP-102®, and Peppermint oil.

Another embodiment comprises cyclosporin A, Nordox OP-114®, and Peppermint oil.

Another embodiment comprises cyclosporin A, Norfox OP-45®, and Peppermint oil.

Another embodiment comprises cyclosporin A, Pegosperse 100-L®, and Peppermint oil.

Another embodiment comprises cyclosporin A, Pegosperse 100-O®, and Peppermint oil.

Another embodiment comprises cyclosporin A, Pegosperse 100-S®, and Peppermint oil.

Another embodiment comprises cyclosporin A, Pegosperse 1500-MS®, and Peppermint oil.

Another embodiment comprises cyclosporin A, Pegosperse 175-MS®, and Peppermint oil.

Another embodiment comprises cyclosporin A, Pegosperse 200 DL®, and Peppermint oil.

Another embodiment comprises cyclosporin A, Pegosperse 200-ML®, and Peppermint oil.

Another embodiment comprises cyclosporin A, Pegosperse 400-DL®, and Peppermint oil.

Another embodiment comprises cyclosporin A, Pegosperse 400-DO®, and Peppermint oil.

Another embodiment comprises cyclosporin A, Pegosperse 400-DS®, and Peppermint oil.

Another embodiment comprises cyclosporin A, Pegosperse 400-ML®, and Peppermint oil.

Another embodiment comprises cyclosporin A, Pegosperse 400-MO®, and Peppermint oil.

Another embodiment comprises cyclosporin A, Pegosperse 400-MOT®, and Peppermint oil.

Another embodiment comprises cyclosporin A, Pegosperse 400-MS®, and Peppermint oil.

Another embodiment comprises cyclosporin A, Pegosperse 50-DS®, and Peppermint oil.

Another embodiment comprises cyclosporin A, Pegosperse 50-MS®, and Peppermint oil.

Another embodiment comprises cyclosporin A, Pegosperse 600-ML®, and Peppermint oil.

Another embodiment comprises cyclosporin A, Pegosperse 600-MS®, and Peppermint oil.

Another embodiment comprises cyclosporin A, Plurafac A-38®, and Peppermint oil.

Another embodiment comprises cyclosporin A, Plurafac B-25-5®, and Peppermint oil.

Another embodiment comprises cyclosporin A, Plurafac B-26®, and Peppermint oil.

Another embodiment comprises cyclosporin A, Plurafac D-25®, and Peppermint oil.

Another embodiment comprises cyclosporin A, Plurafac LF 131®, and Peppermint oil.

Another embodiment comprises cyclosporin A, Plurafac LF 4030®, and Peppermint oil.

Another embodiment comprises cyclosporin A, Plurafac LF 7000®, and Peppermint oil.

Another embodiment comprises cyclosporin A, Plurafac RA-20®, and Peppermint oil.

Another embodiment comprises cyclosporin A, Plurafac RA-30®, and Peppermint oil.

Another embodiment comprises cyclosporin A, Plurafac RA-40®, and Peppermint oil.

Another embodiment comprises cyclosporin A, Pluronic 10R5®, and Peppermint oil.

Another embodiment comprises cyclosporin A, Pluronic 17R2§, and Peppermint oil.

Another embodiment comprises cyclosporin A, Pluronic 17R4®, and Peppermint oil.

Another embodiment comprises cyclosporin A, Pluronic 25R2®, and Peppermint oil.

Another embodiment comprises cyclosporin A, Pluronic 25R4®, and Peppermint oil.

Another embodiment comprises cyclosporin A, Pluronic 31R1®, and Peppermint oil.

Another embodiment comprises cyclosporin A, Pluronic F108®, and Peppermint oil.

Another embodiment comprises cyclosporin A, Pluronic F127®, and Peppermint oil.

Another embodiment comprises cyclosporin A, Pluronic F38®, and Peppermint oil.

Another embodiment comprises cyclosporin A, Pluronic F68®, and Peppermint oil.

Another embodiment comprises cyclosporin A, Pluronic F68LF®, and Peppermint oil.

Another embodiment comprises cyclosporin A, Pluronic F77®, and Peppermint oil.

Another embodiment comprises cyclosporin A, Pluronic F87®, and Peppermint oil.

Another embodiment comprises cyclosporin A, Pluronic F88®, and Peppermint oil.

Another embodiment comprises cyclosporin A, Pluronic F98®, and Peppermint oil.

Another embodiment comprises cyclosporin A, Pluronic L10®, and Peppermint oil.

Another embodiment comprises cyclosporin A, Pluronic L101®, and Peppermint oil.

Another embodiment comprises cyclosporin A, Pluronic L121®, and Peppermint oil.

Another embodiment comprises cyclosporin A, Pluronic L31®, and Peppermint oil.

Another embodiment comprises cyclosporin A, Pluronic L35®, and Peppermint oil.

Another embodiment comprises cyclosporin A, Pluronic L43®, and Peppermint oil.

Another embodiment comprises cyclosporin A, Pluronic L44®, and Peppermint oil.

Another embodiment comprises cyclosporin A, Pluronic L61®, and Peppermint oil.

Another embodiment comprises cyclosporin A, Pluronic L62®, and Peppermint oil.

Another embodiment comprises cyclosporin A, Pluronic L62D®, and Peppermint oil.

Another embodiment comprises cyclosporin A, Pluronic L62LF®, and Peppermint oil.

Another embodiment comprises cyclosporin A, Pluronic L64®, and Peppermint oil.

Another embodiment comprises cyclosporin A, Pluronic L81®, and Peppermint oil.

Another embodiment comprises cyclosporin A, Pluronic L92®, and Peppermint oil.

Another embodiment comprises cyclosporin A, Pluronic P103®, and Peppermint oil.

Another embodiment comprises cyclosporin A, Pluronic P104®, and Peppermint oil.

Another embodiment comprises cyclosporin A, Pluronic P105®, and Peppermint oil.

Another embodiment comprises cyclosporin A, Pluronic P123®, and Peppermint oil.

Another embodiment comprises cyclosporin A, Pluronic P65®, and Peppermint oil.

Another embodiment comprises cyclosporin A, Pluronic P84®, and Peppermint oil.

Another embodiment comprises cyclosporin A, Pluronic P85®, and Peppermint oil.

Another embodiment comprises cyclosporin A, Polyaldo 10-2-P®, and Peppermint oil.

Another embodiment comprises cyclosporin A, Polyaldo DGDO®, and Peppermint oil.

Another embodiment comprises cyclosporin A, Polyaldo HGDS®, and Peppermint oil.

Another embodiment comprises cyclosporin A, Polyaldo TGMS®, and Peppermint oil.

Another embodiment comprises cyclosporin A, Protegin V®, and Peppermint oil.

Another embodiment comprises cyclosporin A, Protegin W®, and Peppermint oil.

Another embodiment comprises cyclosporin A, Protegin WX®, and Peppermint oil.

Another embodiment comprises cyclosporin A, Protegin XV®, and Peppermint oil.

Another embodiment comprises cyclosporin A, Rhodasurf BC-420®, and Peppermint oil.

Another embodiment comprises cyclosporin A, Rhodasurf BC-610®, and Peppermint oil.

Another embodiment comprises cyclosporin A, Rhodasurf BC-630®, and Peppermint oil.

Another embodiment comprises cyclosporin A, Rhodasurf BC-720®, and Peppermint oil.

Another embodiment comprises cyclosporin A, Rhodasurf BC-840®, and Peppermint oil.

Another embodiment comprises cyclosporin A, Rhodasurf L 1®, and Peppermint oil.

Another embodiment comprises cyclosporin A, Rhodasurf L 12®, and Peppermint oil.

Another embodiment comprises cyclosporin A, Rhodasurf L 2®, and Peppermint oil.

Another embodiment comprises cyclosporin A, Rhodasurf L 20®, and Peppermint oil.

Another embodiment comprises cyclosporin A, Rhodasurf L 3®, and Peppermint oil.

Another embodiment comprises cyclosporin A, Rhodasurf L 30®, and Peppermint oil.

Another embodiment comprises cyclosporin A, Rhodasurf L 4®, and Peppermint oil.

Another embodiment comprises cyclosporin A, Rhodasurf L 50®, and Peppermint oil.

Another embodiment comprises cyclosporin A, Rhodasurf L 7/90®, and Peppermint oil.

Another embodiment comprises cyclosporin A, Rhodasurf LA-3®, and Peppermint oil.

Another embodiment comprises cyclosporin A, Rhodasurf LA-7®, and Peppermint oil.

Another embodiment comprises cyclosporin A, Rhodasurf LAN-23®, and Peppermint oil.

Another embodiment comprises cyclosporin A, Rhodasurf ON-870®, and Peppermint oil.

Another embodiment comprises cyclosporin A, Rhodasurf ON-877®, and Peppermint oil.

Another embodiment comprises cyclosporin A, Rhodasurf TDA-8/5®, and Peppermint oil.

Another embodiment comprises cyclosporin A, Ritapro 100®, and Peppermint oil.

Another embodiment comprises cyclosporin A, Ritapro 165®, and Peppermint oil.

Another embodiment comprises cyclosporin A, Ritapro 200®, and Peppermint oil.

Another embodiment comprises cyclosporin A, Ritapro 300®, and Peppermint oil.

Another embodiment comprises cyclosporin A, Ritoleth 10®, and Peppermint oil.

Another embodiment comprises cyclosporin A, Ritoleth 2®, and Peppermint oil.

Another embodiment comprises cyclosporin A, Ritoleth 20®, and Peppermint oil.

Another embodiment comprises cyclosporin A, Ritoleth 5®, and Peppermint oil.

Another embodiment comprises cyclosporin A, Santone 10-10-0®, and Peppermint oil.

Another embodiment comprises cyclosporin A, Santone 3-1-S XTR®, and Peppermint oil.

Another embodiment comprises cyclosporin A, Santone 3-1 -SH®and Peppermint oil.

Another embodiment comprises cyclosporin A, Santone 8-1 -O®, and Peppermint oil.

Another embodiment comprises cyclosporin A, Solulan 16®, and Peppermint oil.

Another embodiment comprises cyclosporin A, Solulan 75®, and Peppermint oil.

Another embodiment comprises cyclosporin A, Solulan C-24®, and Peppermint oil.

Another embodiment comprises cyclosporin A, Solulan L-575®, and Peppermint oil.

Another embodiment comprises cyclosporin A, Standamox CAW®, and Peppermint oil.

Another embodiment comprises cyclosporin A, Sunine AZI-A®, and Peppermint oil.

Another embodiment comprises cyclosporin A, Surfonic CO-15®, and Peppermint oil.

Another embodiment comprises cyclosporin A, Surfonic CO-25®, and Peppermint oil.

Another embodiment comprises cyclosporin A, Surfonic CO-30®, and Peppermint oil.

Another embodiment comprises cyclosporin A, Surfonic CO-36®, and Peppermint oil.

Another embodiment comprises cyclosporin A, Surfonic CO-42®, and Peppermint oil.

Another embodiment comprises cyclosporin A, Surfonic E400-MO®, and Peppermint oil.

Another embodiment comprises cyclosporin A, Surfonic L12-3®, and Peppermint oil.

Another embodiment comprises cyclosporin A, Surfonic L12-6®, and Peppermint oil.

Another embodiment comprises cyclosporin A, Surfonic Li 12-8®, and Peppermint oil.

Another embodiment comprises cyclosporin A, Surfonic L24-12®, and Peppermint oil.

Another embodiment comprises cyclosporin A, Surfonic L24-2®, and Peppermint oil.

Another embodiment comprises cyclosporin A, Surfonic L24-22®, and Peppermint oil.

Another embodiment comprises cyclosporin A, Surfonic L24-3®, and Peppermint oil.

Another embodiment comprises cyclosporin A, Surfonic L24-4®, and Peppermint oil.

Another embodiment comprises cyclosporin A, Surfonic L24-5®, and Peppermint oil.

Another embodiment comprises cyclosporin A, Surfonic L24-7®, and Peppermint oil.

Another embodiment comprises cyclosporin A, Surfonic L24-7.1®, and Peppermint oil.

Another embodiment comprises cyclosporin A, Surfonic L24-9®, and Peppermint oil.

Another embodiment comprises cyclosporin A, Surtonic LF-17®, and Peppermint oil.

Another embodiment comprises cyclosporin A, Surtonic LF-18®, and Peppermint oil.

Another embodiment comprises cyclosporin A, Surtonic LF-37®, and Peppermint oil.

Another embodiment comprises cyclosporin A, Surtonic LF-41®, and Peppermint oil.

Another embodiment comprises cyclosporin A, Surtonic LF-42®, and Peppermint oil.

Another embodiment comprises cyclosporin A, Surtonic LF-47®, and Peppermint oil.

Another embodiment comprises cyclosporin A, Surfonic LF-68,®, and Peppermint oil.

Another embodiment comprises cyclosporin A, Surtonic N-10®, and Peppermint oil.

Another embodiment comprises cyclosporin A, Surfonic N-100®, and Peppermint oil.

Another embodiment comprises cyclosporin A, Surfonic N-102®, and Peppermint oil.

Another embodiment comprises cyclosporin A, Surfonic N-120®, and Peppermint oil.

Another embodiment comprises cyclosporin A, Surfonic N-150®, and Peppermint oil.

Another embodiment comprises cyclosporin A, Surfonic N-200®, and Peppermint oil.

Another embodiment comprises cyclosporin A, Surfonic N-300®, and Peppermint oil.

Another embodiment comprises cyclosporin A, Surfonic N-31.5®, and Peppermint oil.

Another embodiment comprises cyclosporin A, Surfonic N-40®, and Peppermint oil.

Another embodiment comprises cyclosporin A, Surfonic N-400®, and Peppermint oil.

Another embodiment comprises cyclosporin A, Surfonic N-60®, and Peppermint oil.

Another embodiment comprises cyclosporin A, Surfonic N-85®, and Peppermint oil.

Another embodiment comprises cyclosporin A, Surfonic N-95®, and Peppermint oil.

Another embodiment comprises cyclosporin A, Surfonic NB-18®, and Peppermint oil.

Another embodiment comprises cyclosporin A, Surfonic NB-23®, and Peppermint oil.

Another embodiment comprises cyclosporin A, Surfonic NB-25®, and Peppermint oil.

Another embodiment comprises cyclosporin A, Surfonic NB-307®, and Peppermint oil.

Another embodiment comprises cyclosporin A, Surfonic NB-31®, and Peppermint oil.

Another embodiment comprises cyclosporin A, Surfonic NB-41®, and Peppermint oil.

Another embodiment comprises cyclosporin A, Surfonic NB-43®, and Peppermint oil.

Another embodiment comprises cyclosporin A, Surfonic OP-100®, and Peppermint oil.

Another embodiment comprises cyclosporin A, Surfonic OP-120®, and Peppermint oil.

Another embodiment comprises cyclosporin A, Surfonic OP-15®, and Peppermint oil.

Another embodiment comprises cyclosporin A, Surfonic OP-35®, and Peppermint oil.

Another embodiment comprises cyclosporin A, Surfonic OP-70®, and Peppermint oil.

Another embodiment comprises cyclosporin A, Surfonic P1®, and Peppermint oil.

Another embodiment comprises cyclosporin A, Surfonic P3®, and Peppermint oil.

Another embodiment comprises cyclosporin A, Surfonic P5®, and Peppermint oil.

Another embodiment comprises cyclosporin A, Surfonic P6®, and Peppermint oil.

Another embodiment comprises cyclosporin A, Surfonic POA-17R2®, and Peppermint oil.

Another embodiment comprises cyclosporin A, Surfonic POA-25R2®, and Peppermint oil.

Another embodiment comprises cyclosporin A, Surfonic TDA-11®, and Peppermint oil.

Another embodiment comprises cyclosporin A, Surfonic TDA-3B®, and Peppermint oil.

Another embodiment comprises cyclosporin A, Surfonic TDA-6®, and Peppermint oil.

Another embodiment comprises cyclosporin A, Surfonic TDA-6/88®, and Peppermint oil.

Another embodiment comprises cyclosporin A, Surfonic TDA-8®, and Peppermint oil.

Another embodiment comprises cyclosporin A, Surfonic TDA-8/90®, and Peppermint oil.

Another embodiment comprises cyclosporin A, Surfonic TDA-9®, and Peppermint oil.

Another embodiment comprises cyclosporin A, Surfonic TX-CCN®, and Peppermint oil.

Another embodiment comprises cyclosporin A, Tagat CH 40®, and Peppermint oil.

Another embodiment comprises cyclosporin A, Tagat CH 60®, and Peppermint oil.

Another embodiment comprises cyclosporin A, Tagat L 2®, and Peppermint oil.

Another embodiment comprises cyclosporin A, Tagat S®, and Peppermint oil.

Another embodiment comprises cyclosporin A, Tagat S 2®, and Peppermint oil.

Another embodiment comprises cyclosporin A, Tally 100 Plus®, and Peppermint oil.

Another embodiment comprises cyclosporin A, T-Det A106®, and Peppermint oil.

Another embodiment comprises cyclosporin A, T-Det A109®, and Peppermint oil.

Another embodiment comprises cyclosporin A, T-Det Al136®, and Peppermint oil.

Another embodiment comprises cyclosporin A, T-Det Al139®, and Peppermint oil.

Another embodiment comprises cyclosporin A, T-Det A2412®, and Peppermint oil.

Another embodiment comprises cyclosporin A, T-Det A243®, and Peppermint oil.

Another embodiment comprises cyclosporin A, T-Det A247®, and Peppermint oil.

Another embodiment comprises cyclosporin A, T-Det A249®, and Peppermint oil.

Another embodiment comprises cyclosporin A, T-Det A467®, and Peppermint oil.

Another embodiment comprises cyclosporin A, T-Det COE®, and Peppermint oil.

Another embodiment comprises cyclosporin A, T-Det DD-10®, and Peppermint oil.

Another embodiment comprises cyclosporin A, T-Det DD-5®, and Peppermint oil.

Another embodiment comprises cyclosporin A, T-Det DD-7®, and Peppermint oil.

Another embodiment comprises cyclosporin A, T-Det EPO-104®, and Peppermint oil.

Another embodiment comprises cyclosporin A, T-Det EPO-61®, and Peppermint oil.

Another embodiment comprises cyclosporin A, T-Det EPO-62®, and Peppermint oil.

Another embodiment comprises cyclosporin A, T-Det EPO-64®, and Peppermint oil.

Another embodiment comprises cyclosporin A, T-Det N-1.5®, and Peppermint oil.

Another embodiment comprises cyclosporin A, T-Det N-10.5®, and Peppermint oil.

Another embodiment comprises cyclosporin A, T-Det N-100®, and Peppermint oil.

Another embodiment comprises cyclosporin A, T-Det N-1007®, and Peppermint oil.

Another embodiment comprises cyclosporin A, T-Det N-12®, and Peppermint oil.

Another embodiment comprises cyclosporin A, T-Det N-14®, and Peppermint oil.

Another embodiment comprises cyclosporin A, T-Det N-20®, and Peppermint oil.

Another embodiment comprises cyclosporin A, T-Det N-30®, and Peppermint oil.

Another embodiment comprises cyclosporin A, T-Det N-307®, and Peppermint oil.

Another embodiment comprises cyclosporin A, T-Det N-4®, and Peppermint oil.

Another embodiment comprises cyclosporin A, T-Det N-40®, and Peppermint oil.

Another embodiment comprises cyclosporin A, T-Det N-407®, and Peppermint oil.

Another embodiment comprises cyclosporin A, T-Det N-50®, and Peppermint oil.

Another embodiment comprises cyclosporin A, T-Det N-507®, and Peppermint oil.

Another embodiment comprises cyclosporin A, T-Det N-6®, and Peppermint oil.

Another embodiment comprises cyclosporin A, T-Det N-70®, and Peppermint oil.

Another embodiment comprises cyclosporin A, T-Det N-8®, and Peppermint oil.

Another embodiment comprises cyclosporin A, T-Det N-9.5®, and Peppermint oil.

Another embodiment comprises cyclosporin A, T-Det O-12®, and Peppermint oil.

Another embodiment comprises cyclosporin A, T-Det O-165®, and Peppermint oil.

Another embodiment comprises cyclosporin A, T-Det O-307®, and Peppermint oil.

Another embodiment comprises cyclosporin A, T-Det O-40®, and Peppermint oil.

Another embodiment comprises cyclosporin A, T-Det O-407®, and Peppermint oil.

Another embodiment comprises cyclosporin A, T-Det O-6®, and Peppermint oil.

Another embodiment comprises cyclosporin A, T-Det O-8®, and Peppermint oil.

Another embodiment comprises cyclosporin A, T-Det O-9®, and Peppermint oil.

Another embodiment comprises cyclosporin A, Tegine®, and Peppermint oil.

Another embodiment comprises cyclosporin A, Tergitol 15-S-12®, and Peppermint oil.

Another embodiment comprises cyclosporin A, Tergitol 15-S-15®, and Peppermint oil.

Another embodiment comprises cyclosporin A, Tergitol 15-S-20®, and Peppermint oil.

Another embodiment comprises cyclosporin A, Tergitol 15-S-20 (80AQ)®, and Peppermint oil.

Another embodiment comprises cyclosporin A, Tergitol 15-S-3®, and Peppermint oil.

Another embodiment comprises cyclosporin A, Tergitol 15-S-30®, and Peppermint oil.

Another embodiment comprises cyclosporin A, Tergitol 15-S-40®, and Peppermint oil.

Another embodiment comprises cyclosporin A, Tergitol 15-S-40 (70A0)®, and Peppermint oil.

Another embodiment comprises cyclosporin A, Tergitol 15-S-5®, and Peppermint oil.

Another embodiment comprises cyclosporin A, Tergitol 15-S-7®, and Peppermint oil.

Another embodiment comprises cyclosporin A, Tergitol 15-S-9®, and Peppermint oil.

Another embodiment comprises cyclosporin A, Tergitol L-101®, and Peppermint oil.

Another embodiment comprises cyclosporin A, Tergitol L-61®, and Peppermint oil.

Another embodiment comprises cyclosporin A, Tergitol L-62®, and Peppermint oil.

Another embodiment comprises cyclosporin A, Tergitol L-64®, and Peppermint oil.

Another embodiment comprises cyclosporin A, Tergitol L-81®, and Peppermint oil.

Another embodiment comprises cyclosporin A, Tergitol NP-10®, and Peppermint oil.

Another embodiment comprises cyclosporin A, Tergitol NP-11®, and Peppermint oil.

Another embodiment comprises cyclosporin A, Tergitol NP-12®, and Peppermint oil.

Another embodiment comprises cyclosporin A, Tergitol NP-13®, and Peppermint oil.

Another embodiment comprises cyclosporin A, Tergitol NP-15®, and Peppermint oil.

Another embodiment comprises cyclosporin A, Tergitol NP-30®, and Peppermint oil.

Another embodiment comprises cyclosporin A, Tergitol NP-30 (70 AQ)®, and Peppermint oil.

Another embodiment comprises cyclosporin A, Tergitol NP-4®, and Peppermint oil.

Another embodiment comprises cyclosporin A, Tergitol NP-40®, and Peppermint oil.

Another embodiment comprises cyclosporin A, Tergitol NP-40 (70 AQ)®, and Peppermint oil.

Another embodiment comprises cyclosporin A, Tergitol NP-50 (70 AQ)®, and Peppermint oil.

Another embodiment comprises cyclosporin A, Tergitol NP-55 (70 AQ)®, and Peppermint oil.

Another embodiment comprises cyclosporin A, Tergitol NP-6®, and Peppermint oil.

Another embodiment comprises cyclosporin A, Tergitol NP-7®, and Peppermint oil.

Another embodiment comprises cyclosporin A, Tergitol NP-70 (70 AQ)®, and Peppermint oil.

Another embodiment comprises cyclosporin A, Tergitol NP-8®, and Peppermint oil.

Another embodiment comprises cyclosporin A, Tergitol NP-9®, and Peppermint oil.

Another embodiment comprises cyclosporin A, Tergitol NP-9.5®, and Peppermint oil.

Another embodiment comprises cyclosporin A, Tergitol TMN-10®, and Peppermint oil.

Another embodiment comprises cyclosporin A, Tergitol TMN-3®, and Peppermint oil.

Another embodiment comprises cyclosporin A, Tergitol TMN-6®, and Peppermint oil.

Another embodiment comprises cyclosporin A, Tergitol XD®, and Peppermint oil.

Another embodiment comprises cyclosporin A, Tergitol XH®, and Peppermint oil.

Another embodiment comprises cyclosporin A, Tergitol XJ®, and Peppermint oil..

Another embodiment comprises cyclosporin A, Tetronic 1107®, and Peppermint oil.

Another embodiment comprises cyclosporin A, Tetronic 1307®, and Peppermint oil.

Another embodiment comprises cyclosporin A, Tetronic 150R1®, and Peppermint oil.

Another embodiment comprises cyclosporin A, Tetronic 304®, and Peppermint oil.

Another embodiment comprises cyclosporin A, Tetronic 701®, and Peppermint oil.

Another embodiment comprises cyclosporin A, Tetronic 901®, and Peppermint oil.

Another embodiment comprises cyclosporin A, Tetronic 904®, and Peppermint oil.

Another embodiment comprises cyclosporin A, Tetronic 908®, and Peppermint oil.

Another embodiment comprises cyclosporin A, Tetronic 90R4®, and Peppermint oil.

Another embodiment comprises cyclosporin A, Tomadol 1-3®, and Peppermint oil.

Another embodiment comprises cyclosporin A, Tomadol 1-5®, and Peppermint oil.

Another embodiment comprises cyclosporin A, Tomadol 1-7®, and Peppermint oil.

Another embodiment comprises cyclosporin A, Tomadol 23-1®, and Peppermint oil.

Another embodiment comprises cyclosporin A, Tomadol 23-3®, and Peppermint oil.

Another embodiment comprises cyclosporin A, Tomadol 23-6.5®, and Peppermint oil.

Another embodiment comprises cyclosporin A, Tomadol 23-6.5®, and Peppermint oil.

Another embodiment comprises cyclosporin A, Tomadol 25-12®, and Peppermint oil.

Another embodiment comprises cyclosporin A, Tomadol 25-3®, and Peppermint oil.

Another embodiment comprises cyclosporin A, Tomadol 25-7®, and Peppermint oil.

Another embodiment comprises cyclosporin A, Tomadol 25-9®, and Peppermint oil.

Another embodiment comprises cyclosporin A, Tomadol 45-13®, and Peppermint oil.

Another embodiment comprises cyclosporin A, Tomadol 45-2.5®, and Peppermint oil.

Another embodiment comprises cyclosporin A, Tomadol 45-7®, and Peppermint oil.

Another embodiment comprises cyclosporin A, Tomadol 91-2.5®, and Peppermint oil.

Another embodiment comprises cyclosporin A, Tomadol 91-6®, and Peppermint oil.

Another embodiment comprises cyclosporin A, Tomadol 91-8®, and Peppermint oil.

Another embodiment comprises cyclosporin A, Triton X-100®, and Peppermint oil.

Another embodiment comprises cyclosporin A, Triton X-102®, and Peppermint oil.

Another embodiment comprises cyclosporin A, Triton X-114®, and Peppermint oil.

Another embodiment comprises cyclosporin A, Triton X-15®, and Peppermint oil.

Another embodiment comprises cyclosporin A, Triton X-165 (70%)0®, and Peppermint oil.

Another embodiment comprises cyclosporin A, Triton X-207®, and Peppermint oil.

Another embodiment comprises cyclosporin A, Triton X-305 (70%)®, and Peppermint oil.

Another embodiment comprises cyclosporin A, Triton X-35®, and Peppermint oil.

Another embodiment comprises cyclosporin A, Triton X-405 (70%)®, and Peppermint oil.

Another embodiment comprises cyclosporin A, Triton X-45®, and Peppermint oil.

Another embodiment comprises cyclosporin A, Triton X-705 (7O%)®, and Peppermint oil.

Another embodiment comprises cyclosporin A, Volpo S-10®, and Peppermint oil.

Another embodiment comprises cyclosporin A, Volpo S-2®, and Peppermint oil.

Another embodiment comprises cyclosporin A, Volpo S-20®, and Peppermint oil.

Another embodiment comprises cyclosporin A, Volpo-10®, and Peppermint oil.

Another embodiment comprises cyclosporin A, Volpo-20®, and Peppermint oil.

Another embodiment comprises cyclosporin A, Volpo-3®, and Peppermint oil.

Another embodiment comprises cyclosporin A, Volpo-5®, and Peppermint oil.

Another embodiment comprises cyclosporin A, Abex 2515®, and Sunflower oil.

Another embodiment comprises cyclosporin A, Abex 2525/40®, and Sunflower oil.

Another embodiment comprises cyclosporin A, Abex 2535®, and Sunflower oil.

Another embodiment comprises cyclosporin A, Abex 2545®, and Sunflower oil.

Another embodiment comprises cyclosporin A, Acconon E®, and Sunflower oil.

Another embodiment comprises cyclosporin A, Alfonic 1216CO-9®, and Sunflower oil.

Another embodiment comprises cyclosporin A, Alfonic 1412-3®, and Sunflower oil.

Another embodiment comprises cyclosporin A, Alfonic TDA-12®, and Sunflower oil.

Another embodiment comprises cyclosporin A, Alfonic TDA-3®, and Sunflower oil.

Another embodiment comprises cyclosporin A, Alfonic TDA-4®, and Sunflower oil.

Another embodiment comprises cyclosporin A, Alfonic TDA-6®, and Sunflower oil.

Another embodiment comprises cyclosporin A, Alfonic TDA-7®, and Sunflower oil.

Another embodiment comprises cyclosporin A, Alfonic TDA-8®, and Sunflower oil.

Another embodiment comprises cyclosporin A, Alfonic TDA-9®, and Sunflower oil.

Another embodiment comprises cyclosporin A, Alkamuls 400-DO®, and Sunflower oil.

Another embodiment comprises cyclosporin A, Alkamuls 400-MO/E®, and Sunflower oil.

Another embodiment comprises cyclosporin A, Ameroxol OE-10®, and Sunflower oil.

Another embodiment comprises cyclosporin A, Ameroxol OE-2®, and Sunflower oil.

Another embodiment comprises cyclosporin A, Ameroxol OE-20®, and Sunflower oil.

Another embodiment comprises cyclosporin A, Armix 176®, and Sunflower oil.

Another embodiment comprises cyclosporin A, Armix 180-C®, and Sunflower oil.

Another embodiment comprises cyclosporin A, Armix 183®, and Sunflower oil.

Another embodiment comprises cyclosporin A, Brij 35®, and Sunflower oil:

Another embodiment comprises cyclosporin A, Brij 35 Liquid®, and Sunflower oil.

Another embodiment comprises cyclosporin A, Brij 35 SP®, and Sunflower oil.

Another embodiment comprises cyclosporin A, Canasol MJ 2109®, and Sunflower oil.

Another embodiment comprises cyclosporin A, Canasol MJ 52®, and Sunflower oil.

Another embodiment comprises cyclosporin A, Canasol R 3600®, and Sunflower oil.

Another embodiment comprises cyclosporin A, Canasol R 3603®, and Sunflower oil.

Another embodiment comprises cyclosporin A, Canasol R 4000 H®, and Sunflower oil.

Another embodiment comprises cyclosporin A, Cedepal CA-210®, and Sunflower oil.

Another embodiment comprises cyclosporin A, Cedepal CA-520®, and Sunflower oil.

Another embodiment comprises cyclosporin A, Cedepal CA-630®, and Sunflower oil.

Another embodiment comprises cyclosporin A, Cedepal CA-720®, and Sunflower oil.

Another embodiment comprises cyclosporin A, Cedepal CA-890®, and Sunflower oil.

Another embodiment comprises cyclosporin A, Cedepal CA-897®, and Sunflower oil.

Another embodiment comprises cyclosporin A, Cedepal CO-210®, and Sunflower oil.

Another embodiment comprises cyclosporin A, Cedepal CO-430®, and Sunflower oil.

Another embodiment comprises cyclosporin A, Cedepal CO-530®, and Sunflower oil.

Another embodiment comprises cyclosporin A, Cedepal CO-630®, and Sunflower oil.

Another embodiment comprises cyclosporin A, Cedepal CO-710®, and Sunflower oil.

Another embodiment comprises cyclosporin A, Cedepal CO-730®, and Sunflower oil.

Another embodiment comprises cyclosporin A, Cedepal CO-880®, and Sunflower oil.

Another embodiment comprises cyclosporin A, Cedepal CO-887®, and Sunflower oil.

Another embodiment comprises cyclosporin A, Cedepal CO-890®, and Sunflower oil.

Another embodiment comprises cyclosporin A, Cedepal CO-897®, and Sunflower oil.

Another embodiment comprises cyclosporin A, Cedepal CO-970®, and Sunflower oil.

Another embodiment comprises cyclosporin A, Cedepal CO-977®, and Sunflower oil.

Another embodiment comprises cyclosporin A, Cedepal CO-990®, and Sunflower oil.

Another embodiment comprises cyclosporin A, Cedepal CO-997®, and Sunflower oil.

Another embodiment comprises cyclosporin A, Chemal BP 261®, and Sunflower oil.

Another embodiment comprises cyclosporin A, Chemal BP 262®, and Sunflower oil.

Another embodiment comprises cyclosporin A, Chemal BP 3172®, and Sunflower oil.

Another embodiment comprises cyclosporin A, Chemal BP 3174®, and Sunflower oil.

Another embodiment comprises cyclosporin A, Chemal DA-4®, and Sunflower oil.

Another embodiment comprises cyclosporin A, Chemal DA-6®, and Sunflower oil.

Another embodiment comprises cyclosporin A, Chemal DA-9®, and Sunflower oil.

Another embodiment comprises cyclosporin A, Chemal LA-23®, and Sunflower oil.

Another embodiment comprises cyclosporin A, Chemal LA-4®, and Sunflower oil.

Another embodiment comprises cyclosporin A, Chemal LA-9®, and Sunflower oil.

Another embodiment comprises cyclosporin A, Chemal OA-20G®, and Sunflower oil.

Another embodiment comprises cyclosporin A, Chemal OA-5®, and Sunflower oil.

Another embodiment comprises cyclosporin A, Chemal OA-9®, and Sunflower oil.

Another embodiment comprises cyclosporin A, Chemal TDA-12®, and Sunflower oil.

Another embodiment comprises cyclosporin A, Chemal TDA-15®, and Sunflower oil.

Another embodiment comprises cyclosporin A, Chemal TDA-3®, and Sunflower oil.

Another embodiment comprises cyclosporin A, Chemal TDA-6®, and Sunflower oil.

Another embodiment comprises cyclosporin A, Chemal TDA-9®, and Sunflower oil.

Another embodiment comprises cyclosporin A, Chemax CO-16®, and Sunflower oil.

Another embodiment comprises cyclosporin A, Chemax CO-200/50®, and Sunflower oil.

Another embodiment comprises cyclosporin A, Chemax CO-25®, and Sunflower oil.

Another embodiment comprises cyclosporin A, Chemax CO-30®, and Sunflower oil.

Another embodiment comprises cyclosporin A, Chemax CO-36®, and Sunflower oil.

Another embodiment comprises cyclosporin A, Chemax CO-40®, and Sunflower oil.

Another embodiment comprises cyclosporin A, Chemax CO-5®, and Sunflower oil.

Another embodiment comprises cyclosporin A, Chemax CO-80®, and Sunflower oil.

Another embodiment comprises cyclosporin A, Chemax DNP-15/50®, and Sunflower oil.

Another embodiment comprises cyclosporin A, Chemax DNP-18®, and Sunflower oil.

Another embodiment comprises cyclosporin A, Chemax DNP-8®, and Sunflower oil.

Another embodiment comprises cyclosporin A, Chemax E 200 ML®, and Sunflower oil.

Another embodiment comprises cyclosporin A, Chemax E 200 MO®, and Sunflower oil.

Another embodiment comprises cyclosporin A, Chemax E 400 ML®, and Sunflower oil.

Another embodiment comprises cyclosporin A, Chemax E 400 MO®, and Sunflower oil.

Another embodiment comprises cyclosporin A, Chemax E 600 ML®, and Sunflower oil.

Another embodiment comprises cyclosporin A, Chemax E 600 MO®, and Sunflower oil.

Another embodiment comprises cyclosporin A, Chemax E 1000 MO®, and Sunflower oil.

Another embodiment comprises cyclosporin A, Chemax E 1000 MS®, and Sunflower oil.

Another embodiment comprises cyclosporin A, Chemax E 200 MS®, and Sunflower oil.

Another embodiment comprises cyclosporin A, Chemax E 400 MS®, and Sunflower oil.

Another embodiment comprises cyclosporin A, Chemax E 600 MS®, and Sunflower oil.

Another embodiment comprises cyclosporin A, Chemax HCO-16®, and Sunflower oil.

Another embodiment comprises cyclosporin A, Chemax HCO-200/50®, and Sunflower oil.

Another embodiment comprises cyclosporin A, Chemax HCO-25®, and Sunflower oil.

Another embodiment comprises cyclosporin A, Chemax HCO-5®, and Sunflower oil.

Another embodiment comprises cyclosporin A, Chemax TO-16®, and Sunflower oil.

Another embodiment comprises cyclosporin A, Chemax TO-8®, and Sunflower oil.

Another embodiment comprises cyclosporin A, Colalube 3409®, and Sunflower oil.

Another embodiment comprises cyclosporin A, Colalube 3411®, and Sunflower oil.

Another embodiment comprises cyclosporin A, Colalube 3413®, and Sunflower oil.

Another embodiment comprises cyclosporin A, Colalube 3414®, and Sunflower oil.

Another embodiment comprises cyclosporin A, Colalube 3415®, and Sunflower oil.

Another embodiment comprises cyclosporin A, Colalube 3416®, and Sunflower oil.

Another embodiment comprises cyclosporin A, Colalube 3417®, and Sunflower oil.

Another embodiment comprises cyclosporin A, Delonic NPE-100®, and Sunflower oil.

Another embodiment comprises cyclosporin A, Delonic NPE-30®, and Sunflower oil.

Another embodiment comprises cyclosporin A, Delonic NPE-40®, and Sunflower oil.

Another embodiment comprises cyclosporin A, Delonic OPE-10®, and Sunflower oil.

Another embodiment comprises cyclosporin A, Delonic OPE-12®, and Sunflower oil.

Another embodiment comprises cyclosporin A, Delonic OPE-30®, and Sunflower oil.

Another embodiment comprises cyclosporin A, Delonic OPE-40®, and Sunflower oil.

Another embodiment comprises cyclosporin A, Delonic OPE-5®, and Sunflower oil.

Another embodiment comprises cyclosporin A, Delonic OPE-7.5®, and Sunflower oil.

Another embodiment comprises cyclosporin A, DePeg 16-CO®, and Sunflower oil.

Another embodiment comprises cyclosporin A, DePeg 25-CO®, and Sunflower oil.

Another embodiment comprises cyclosporin A, DePeg 30-CO®, and Sunflower oil.

Another embodiment comprises cyclosporin A, DePeg 40-CO®, and Sunflower oil.

Another embodiment comprises cyclosporin A, DePeg 5-CO®, and Sunflower oil.

Another embodiment comprises cyclosporin A, DePeg 8-CO®, and Sunflower oil.

Another embodiment comprises cyclosporin A, DeSonic S-100®, and Sunflower oil.

Another embodiment comprises cyclosporin A, DeSonic S-114®, and Sunflower oil.

Another embodiment comprises cyclosporin A, DeSonic S-405®, and Sunflower oil.

Another embodiment comprises cyclosporin A, DeSonic S-45®, and Sunflower oil.

Another embodiment comprises cyclosporin A, DeSonic TDA-9®, and Sunflower oil.

Another embodiment comprises cyclosporin A, DeThox Acid O-14®, and Sunflower oil.

Another embodiment comprises cyclosporin A, DeThox Acid O-16®, and Sunflower oil.

Another embodiment comprises cyclosporin A, DeThox Acid O-9®, and Sunflower oil.

Another embodiment comprises cyclosporin A, DeThox Acid TO-14®, and Sunflower oil.

Another embodiment comprises cyclosporin A, DeThox Acid TO-16.5®, and Sunflower oil.

Another embodiment comprises cyclosporin A, DeThox Acid TO-8.5®, and Sunflower oil.

Another embodiment comprises cyclosporin A, Dur-Em 114®, and Sunflower oil.

Another embodiment comprises cyclosporin A, Dur-Em 117®, and Sunflower oil.

Another embodiment comprises cyclosporin A, Dur-Em 204®, and Sunflower oil.

Another embodiment comprises cyclosporin A, Dur-Em 207®, and Sunflower oil.

Another embodiment comprises cyclosporin A, Dur-Em 207E®, and Sunflower oil.

Another embodiment comprises cyclosporin A, Emerest 2704®, and Sunflower oil.

Another embodiment comprises cyclosporin A, Emerest 2712®, and Sunflower oil.

Another embodiment comprises cyclosporin A, Emerest 2715®, and Sunflower oil.

Another embodiment comprises cyclosporin A, Ethal EH-2®, and Sunflower oil.

Another embodiment comprises cyclosporin A, Ethal EH-5®, and Sunflower oil.

Another embodiment comprises cyclosporin A, Ethal OA-23®, and Sunflower oil.

Another embodiment comprises cyclosporin A, Ethosperse CA-20®, and Sunflower oil.

Another embodiment comprises cyclosporin A, Ethosperse LA-12®, and Sunflower oil.

Another embodiment comprises cyclosporin A, Ethosperse LA-23®, and Sunflower oil.

Another embodiment comprises cyclosporin A, Ethosperse LA-4®, and Sunflower oil.

Another embodiment comprises cyclosporin A, Ethox 3095®, and Sunflower oil.

Another embodiment comprises cyclosporin A, Ethox CO-16®, and Sunflower oil.

Another embodiment comprises cyclosporin A, Ethox CO-200®, and Sunflower oil.

Another embodiment comprises cyclosporin A, Ethox CO-25®, and Sunflower oil.

Another embodiment comprises cyclosporin A, Ethox CO-30®, and Sunflower oil.

Another embodiment comprises cyclosporin A, Ethox CO-36®, and Sunflower oil.

Another embodiment comprises cyclosporin A, Ethox CO-40®, and Sunflower oil.

Another embodiment comprises cyclosporin A, Ethox CO-5®, and Sunflower oil.

Another embodiment comprises cyclosporin A, Ethox DO-14®, and Sunflower oil.

Another embodiment comprises cyclosporin A, Ethox DO-9®, and Sunflower oil.

Another embodiment comprises cyclosporin A, Ethox MA-8®, and Sunflower oil.

Another embodiment comprises cyclosporin A, Ethox Ml-14®, and Sunflower oil.

Another embodiment comprises cyclosporin A, Ethox ML-14®, and Sunflower oil.

Another embodiment comprises cyclosporin A, Ethox ML-5®, and Sunflower oil.

Another embodiment comprises cyclosporin A, Ethox MO-14®, and Sunflower oil.

Another embodiment comprises cyclosporin A, Ethox MO-9®, and Sunflower oil.

Another embodiment comprises cyclosporin A, Ethox MS-14®, and Sunflower oil.

Another embodiment comprises cyclosporin A, Ethox MS-23®, and Sunflower oil.

Another embodiment comprises cyclosporin A, Ethox MS-40®, and Sunflower oil.

Another embodiment comprises cyclosporin A, Ethox MS-8®, and Sunflower oil.

Another embodiment comprises cyclosporin A, Ethox TO-16®, and Sunflower oil.

Another embodiment comprises cyclosporin A, Ethox TO-8®, and Sunflower oil.

Another embodiment comprises cyclosporin A, Ethylan SN-12®, and Sunflower oil.

Another embodiment comprises cyclosporin A, Ethylan SN-70®, and Sunflower oil.

Another embodiment comprises cyclosporin A, Ethylan SN-90®, and Sunflower oil.

Another embodiment comprises cyclosporin A, Flexricin 13®, and Sunflower oil.

Another embodiment comprises cyclosporin A, Flexricin 15®, and Sunflower oil.

Another embodiment comprises cyclosporin A, Genapol 1261®, and Sunflower oil.

Another embodiment comprises cyclosporin A, Generol 122 E25®, and Sunflower oil.

Another embodiment comprises cyclosporin A, Glicepol 160®, and Sunflower oil.

Another embodiment comprises cyclosporin A, Glicepol 560®, and Sunflower oil.

Another embodiment comprises cyclosporin A, Glicepol GMS 20®, and Sunflower oil.

Another embodiment comprises cyclosporin A, Hetoxide BN-13®, and Sunflower oil.

Another embodiment comprises cyclosporin A, Hetoxide DNP-4®, and Sunflower oil.

Another embodiment comprises cyclosporin A, Hetoxide DNP-9.6®, and Sunflower oil.

Another embodiment comprises cyclosporin A, Hetoxide G-26®, and Sunflower oil.

Another embodiment comprises cyclosporin A, Hetoxide G-7®, and Sunflower oil.

Another embodiment comprises cyclosporin A, Hetoxide GT-20®, and Sunflower oil.

Another embodiment comprises cyclosporin A, Hetoxide HC-40®, and Sunflower oil.

Another embodiment comprises cyclosporin A, Hetoxol C-24®, and Sunflower oil.

Another embodiment comprises cyclosporin A, Hetoxol CA-10®, and Sunflower oil.

Another embodiment comprises cyclosporin A, Hetoxol CA-2®, and Sunflower oil.

Another embodiment comprises cyclosporin A, Hetoxol CA-20®, and Sunflower oil.

Another embodiment comprises cyclosporin A, Hetoxol CAWS®, and Sunflower oil.

Another embodiment comprises cyclosporin A, Hetoxol CD-3®, and Sunflower oil.

Another embodiment comprises cyclosporin A, Hetoxol CD-4®, and Sunflower oil.

Another embodiment comprises cyclosporin A, Hetoxol CS-15®, and Sunflower oil.

Another embodiment comprises cyclosporin A, Hetoxol CS-20®, and Sunflower oil.

Another embodiment comprises cyclosporin A, Hetoxol CS-20D®, and Sunflower oil.

Another embodiment comprises cyclosporin A, Hetoxol CS-25®, and Sunflower oil.

Another embodiment comprises cyclosporin A, Hetoxol CS-50®, and Sunflower oil.

Another embodiment comprises cyclosporin A, Hetoxol CS-9®, and Sunflower oil.

Another embodiment comprises cyclosporin A, Hetoxol CSA-15®, and Sunflower oil.

Another embodiment comprises cyclosporin A, Hetoxol D®, and Sunflower oil.

Another embodiment comprises cyclosporin A, Hetoxol G®, and Sunflower oil.

Another embodiment comprises cyclosporin A, Hetoxol J®, and Sunflower oil.

Another embodiment comprises cyclosporin A, Hetoxol L®, and Sunflower oil.

Another embodiment comprises cyclosporin A, Hetoxol L-23®, and Sunflower oil.

Another embodiment comprises cyclosporin A, Hetoxol L-4®, and Sunflower oil.

Another embodiment comprises cyclosporin A, Hetoxol L-9®, and Sunflower oil.

Another embodiment comprises cyclosporin A, Hetoxol LS-9®, and Sunflower oil.

Another embodiment comprises cyclosporin A, Hetoxol M-3®, and Sunflower oil.

Another embodiment comprises cyclosporin A, Hetoxol OA-10 Special®, and Sunflower oil.

Another embodiment comprises cyclosporin A, Hetoxol OA-20 Special®, and Sunflower oil.

Another embodiment comprises cyclosporin A, Hetoxol OA-3 Special®, and Sunflower oil.

Another embodiment comprises cyclosporin A, Hetoxol OA-5 Special®, and Sunflower oil.

Another embodiment comprises cyclosporin A, Hetoxol OL-10®, and Sunflower oil.

Another embodiment comprises cyclosporin A, Hetoxol OL-2®, and Sunflower oil.

Another embodiment comprises cyclosporin A, Hetoxol OL-23®, and Sunflower oil.

Another embodiment comprises cyclosporin A, Hetoxol OL-4®, and Sunflower oil.

Another embodiment comprises cyclosporin A, Hetoxol OL-40®, and Sunflower oil.

Another embodiment comprises cyclosporin A, Hetoxol OL-5®, and Sunflower oil.

Another embodiment comprises cyclosporin A, Hetoxol PLA®, and Sunflower oil.

Another embodiment comprises cyclosporin A, Hetoxol SP-15®, and Sunflower oil.

Another embodiment comprises cyclosporin A, Hetoxol STA-10®, and Sunflower oil.

Another embodiment comprises cyclosporin A, Hetoxol STA-2®, and Sunflower oil.

Another embodiment comprises cyclosporin A, Hetoxol STA-30®, and Sunflower oil.

Another embodiment comprises cyclosporin A, Hetoxol TD-12®, and Sunflower oil.

Another embodiment comprises cyclosporin A, Hetoxol TD-18®, and Sunflower oil.

Another embodiment comprises cyclosporin A, Hetoxol TD-3®, and Sunflower oil.

Another embodiment comprises cyclosporin A, Hetoxol TD-6®, and Sunflower oil.

Another embodiment comprises cyclosporin A, Hetoxol TD-9®, and Sunflower oil.

Another embodiment comprises cyclosporin A, Hetoxol TDEP-15®, and Sunflower oil.

Another embodiment comprises cyclosporin A, Hetoxol TDEP-63®, and Sunflower oil.

Another embodiment comprises cyclosporin A, Iconol 24-12®, and Sunflower oil.

Another embodiment comprises cyclosporin A, Iconol 24-23®, and Sunflower oil.

Another embodiment comprises cyclosporin A, Iconol 24-4®, and Sunflower oil.

Another embodiment comprises cyclosporin A, Iconol DA-4®, and Sunflower oil.

Another embodiment comprises cyclosporin A, Iconol DA-6®, and Sunflower oil.

Another embodiment comprises cyclosporin A, Iconol TDA-10®, and Sunflower oil.

Another embodiment comprises cyclosporin A, Iconol TDA-3®, and Sunflower oil.

Another embodiment comprises cyclosporin A, Iconol TDA-6®, and Sunflower oil.

Another embodiment comprises cyclosporin A, Iconol TDA-8-90%®, and Sunflower oil.

Another embodiment comprises cyclosporin A, Iconol TDA-9®, and Sunflower oil.

Another embodiment comprises cyclosporin A, Igepal CA-210®, and Sunflower oil.

Another embodiment comprises cyclosporin A, Igepal CA-407®, and Sunflower oil.

Another embodiment comprises cyclosporin A, Igepal CA-420®, and Sunflower oil.

Another embodiment comprises cyclosporin A, Igepal CA-520®, and Sunflower oil.

Another embodiment comprises cyclosporin A, Igepal CA-620®, and Sunflower oil.

Another embodiment comprises cyclosporin A, Igepal CA-630®, and Sunflower oil.

Another embodiment comprises cyclosporin A, Igepal CA-720®, and Sunflower oil.

Another embodiment comprises cyclosporin A, Igepal CA-877®, and Sunflower oil.

Another embodiment comprises cyclosporin A, Igepal CA-887®, and Sunflower oil.

Another embodiment comprises cyclosporin A, Igepal CA-897®, and Sunflower oil.

Another embodiment comprises cyclosporin A, Igepal CO-210®, and Sunflower oil.

Another embodiment comprises cyclosporin A, Igepal CO-430®, and Sunflower oil.

Another embodiment comprises cyclosporin A, Igepal CO-520®, and Sunflower oil.

Another embodiment comprises cyclosporin A, Igepal CO-530®, and Sunflower oil.

Another embodiment comprises cyclosporin A, Igepal CO-610®, and Sunflower oil.

Another embodiment comprises cyclosporin A, Igepal CO-630®, and Sunflower oil.

Another embodiment comprises cyclosporin A, Igepal CO-660 Special®, and Sunflower oil.

Another embodiment comprises cyclosporin A, Igepal CO-660®, and Sunflower oil.

Another embodiment comprises cyclosporin A, Igepal CO-710®, and Sunflower oil.

Another embodiment comprises cyclosporin A, Igepal CO-720®, and Sunflower oil.

Another embodiment comprises cyclosporin A, Igepal CO-730®, and Sunflower oil.

Another embodiment comprises cyclosporin A, Igepal CO-738®, and Sunflower oil.

Another embodiment comprises cyclosporin A, Igepal CO-850®, and Sunflower oil.

Another embodiment comprises cyclosporin A, Igepal CO-880®, and Sunflower oil.

Another embodiment comprises cyclosporin A, Igepal CO-887®, and Sunflower oil.

Another embodiment comprises cyclosporin A, Igepal CO-890®, and Sunflower oil.

Another embodiment comprises cyclosporin A, Igepal CO-897®, and Sunflower oil.

Another embodiment comprises cyclosporin A, Igepal CO-970®, and Sunflower oil.

Another embodiment comprises cyclosporin A, Igepal CO-977®, and Sunflower oil.

Another embodiment comprises cyclosporin A, Igepal CO-987®, and Sunflower oil.

Another embodiment comprises cyclosporin A, Igepal CO-990 FLK®, and Sunflower oil.

Another embodiment comprises cyclosporin A, Igepal CO-997®, and Sunflower oil.

Another embodiment comprises cyclosporin A, Igepal DM-430®, and Sunflower oil.

Another embodiment comprises cyclosporin A, Igepal DM-530®, and Sunflower oil.

Another embodiment comprises cyclosporin A, Igepal DM-710®, and Sunflower oil.

Another embodiment comprises cyclosporin A, Igepal DM-730®, and Sunflower oil.

Another embodiment comprises cyclosporin A, Igepal DM-880®, and Sunflower oil.

Another embodiment comprises cyclosporin A, Igepal DM-970 FLK®, and Sunflower oil.

Another embodiment comprises cyclosporin A, Igepal OD-410®, and Sunflower oil.

Another embodiment comprises cyclosporin A, Igepal RC-520®, and Sunflower oil.

Another embodiment comprises cyclosporin A, Igepal RC-620®, and Sunflower oil.

Another embodiment comprises cyclosporin A, Igepal RC-630®, and Sunflower oil.

Another embodiment comprises cyclosporin A, Lexemul CS-20®, and Sunflower oil.

Another embodiment comprises cyclosporin A, Lipo GMS 450®, and Sunflower oil.

Another embodiment comprises cyclosporin A, Lipo Polyglycol 1000®, and Sunflower oil.

Another embodiment comprises cyclosporin A, Lipo Polyglycol 200®, and Sunflower oil.

Another embodiment comprises cyclosporin A, Lipo Polyglycol 300®, and Sunflower oil.

Another embodiment comprises cyclosporin A, Lipo Polyglycol 3350®, and Sunflower oil.

Another embodiment comprises cyclosporin A, Lipo Polyglycol 400®, and Sunflower oil.

Another embodiment comprises cyclosporin A, Lipo Polyglycol 600®, and Sunflower oil.

Another embodiment comprises cyclosporin A, Lipocol C-10®, and Sunflower oil.

Another embodiment comprises cyclosporin A, Lipocol C-2®, and Sunflower oil.

Another embodiment comprises cyclosporin A, Lipocol C-20®, and Sunflower oil.

Another embodiment comprises cyclosporin A, Lipocol L-12®, and Sunflower oil.

Another embodiment comprises cyclosporin A, Lipocol L-23®, and Sunflower oil.

Another embodiment comprises cyclosporin A, Lipocol L-4®, and Sunflower oil.

Another embodiment comprises cyclosporin A, Lipocol O-10®, and Sunflower oil.

Another embodiment comprises cyclosporin A, Lipocol O-2®, and Sunflower oil.

Another embodiment comprises cyclosporin A, Lipocol O-20®, and Sunflower oil.

Another embodiment comprises cyclosporin A, Lipocol O-5®, and Sunflower oil.

Another embodiment comprises cyclosporin A, Lipocol S-10®, and Sunflower oil.

Another embodiment comprises cyclosporin A, Lipocol S-2®, and Sunflower oil.

Another embodiment comprises cyclosporin A, Lipocol S-20®, and Sunflower oil.

Another embodiment comprises cyclosporin A, Lipocol SC-15®, and Sunflower oil.

Another embodiment comprises cyclosporin A, Lipocol SC-20®, and Sunflower oil.

Another embodiment comprises cyclosporin A, Lipocol SC-4®, and Sunflower oil.

Another embodiment comprises cyclosporin A, Lipocol TD-12®, and Sunflower oil.

Another embodiment comprises cyclosporin A, Lumulse 40-O K®, and Sunflower oil.

Another embodiment comprises cyclosporin A, Lumulse 42-O K®, and Sunflower oil.

Another embodiment comprises cyclosporin A, Lumulse GMT-40®, and Sunflower oil.

Another embodiment comprises cyclosporin A, Lumulse PEG 1450®, and Sunflower oil.

Another embodiment comprises cyclosporin A, Lumulse PEG 1450 NF®, and Sunflower oil.

Another embodiment comprises cyclosporin A, Lumulse PEG 8000®, and Sunflower oil.

Another embodiment comprises cyclosporin A, Lumulse PGO®, and Sunflower oil.

Another embodiment comprises cyclosporin A, Lumulse POE (2) Oleyl Amine®, and Sunflower oil.

Another embodiment comprises cyclosporin A, Magrabar PGO-104®, and Sunflower oil.

Another embodiment comprises cyclosporin A, Mapeg 200 ML®, and Sunflower oil.

Another embodiment comprises cyclosporin A, Mapeg 400 MO®, and Sunflower oil.

Another embodiment comprises cyclosporin A, Mapeg 400 MOT®, and Sunflower oil.

Another embodiment comprises cyclosporin A, Mapeg 400DO®, and Sunflower oil.

Another embodiment comprises cyclosporin A, Mapeg 400DOT®, and Sunflower oil.

Another embodiment comprises cyclosporin A, Mapeg 600DOT®, and Sunflower oil.

Another embodiment comprises cyclosporin A, Marlox B 24/80®, and Sunflower oil.

Another embodiment comprises cyclosporin A, Marlox FK 86®, and Sunflower oil.

Another embodiment comprises cyclosporin A, Marlox MO 154®, and Sunflower oil.

Another embodiment comprises cyclosporin A, Mazox CDA CG®, and Sunflower oil.

Another embodiment comprises cyclosporin A, Monoglycerides Glyceryl Monestearate®, and Sunflower oil.

Another embodiment comprises cyclosporin A, Myrj 45®, and Sunflower oil.

Another embodiment comprises cyclosporin A, Myrj 52®, and Sunflower oil.

Another embodiment comprises cyclosporin A, Myrj 52S®, and Sunflower oil.

Another embodiment comprises cyclosporin A, Myrj 53®, and Sunflower oil.

Another embodiment comprises cyclosporin A, Myrj 59®, and Sunflower oil.

Another embodiment comprises cyclosporin A, Norfox NP-12®, and Sunflower oil.

Another embodiment comprises cyclosporin A, Norfox NP-4®, and Sunflower oil.

Another embodiment comprises cyclosporin A, Norfox NP-6®, and Sunflower oil.

Another embodiment comprises cyclosporin A, Norfox NP-887®, and Sunflower oil.

Another embodiment comprises cyclosporin A, Norfox NP-9®, and Sunflower oil.

Another embodiment comprises cyclosporin A, Norfox NP-977®, and Sunflower oil.

Another embodiment comprises cyclosporin A, Norfox OP-100®, and Sunflower oil.

Another embodiment comprises cyclosporin A, Norfox OP-102®, and Sunflower oil.

Another embodiment comprises cyclosporin A, Norfox OP-114®, and Sunflower oil.

Another embodiment comprises cyclosporin A, Norfox OP-45®, and Sunflower oil.

Another embodiment comprises cyclosporin A, Pegosperse 100 -L®, and Sunflower oil.

Another embodiment comprises cyclosporin A, Pegosperse 100-O®, and Sunflower oil.

Another embodiment comprises cyclosporin A, Pegosperse 100S®, and Sunflower oil.

Another embodiment comprises cyclosporin A, Pegosperse 1500-MS®, and Sunflower oil.

Another embodiment comprises cyclosporin A, Pegosperse 1750-MS®, and Sunflower oil.

Another embodiment comprises cyclosporin A, Pegosperse 200 DL®, and Sunflower oil.

Another embodiment comprises cyclosporin A, Pegosperse 200-ML®, and Sunflower oil.

Another embodiment comprises cyclosporin A, Pegosperse 400-DL®, and Sunflower oil.

Another embodiment comprises cyclosporin A, Pegosperse 400-DO®, and Sunflower oil.

Another embodiment comprises cyclosporin A, Pegosperse 400-DS®, and Sunflower oil.

Another embodiment comprises cyclosporin A, Pegosperse 400-ML®, and Sunflower oil.

Another embodiment comprises cyclosporin A, Pegosperse 400-MO®, and Sunflower oil.

Another embodiment comprises cyclosporin A, Pegosperse 400-MOT®, and Sunflower oil.

Another embodiment comprises cyclosporin A, Pegosperse 400-MS®, and Sunflower oil.

Another embodiment comprises cyclosporin A, Pegosperse 50-DS®, and Sunflower oil.

Another embodiment comprises cyclosporin A, Pegosperse 50-MS®, and Sunflower oil.

Another embodiment comprises cyclosporin A, Pegosperse 600-ML®, and Sunflower oil.

Another embodiment comprises cyclosporin A, Pegosperse 600-MS®, and Sunflower oil.

Another embodiment comprises cyclosporin A, Plurafac A-38®, and Sunflower oil.

Another embodiment comprises cyclosporin A, Plurafac B-25-5®, and Sunflower oil.

Another embodiment comprises cyclosporin A, Plurafac B-26®, and Sunflower oil.

Another embodiment comprises cyclosporin A, Plurafac D-25®, and Sunflower oil.

Another embodiment comprises cyclosporin A, Plurafac LF 131®, and Sunflower oil.

Another embodiment comprises cyclosporin A, Plurafac LF 4030®, and Sunflower oil.

Another embodiment comprises cyclosporin A, Plurafac LF 7000®, and Sunflower oil.

Another embodiment comprises cyclosporin A, Plurafac RA-20®, and Sunflower oil.

Another embodiment comprises cyclosporin A, Plurafac RA-30®, and Sunflower oil.

Another embodiment comprises cyclosporin A, Plurafac RA-40®, and Sunflower oil.

Another embodiment comprises cyclosporin A, Pluronic 10R5®, and Sunflower oil.

Another embodiment comprises cyclosporin A, Pluronic 17R2®, and Sunflower oil.

Another embodiment comprises cyclosporin A, Pluronic 17R4®, and Sunflower oil.

Another embodiment comprises cyclosporin A, Pluronic 25R2®, and Sunflower oil.

Another embodiment comprises cyclosporin A, Pluronic 25R4®, and Sunflower oil.

Another embodiment comprises cyclosporin A, Pluronic 31R1®, and Sunflower oil.

Another embodiment comprises cyclosporin A, Pluronic F108®, and Sunflower oil.

Another embodiment comprises cyclosporin A, Pluronic F127®, and Sunflower oil.

Another embodiment comprises cyclosporin A, Pluronic F38®, and Sunflower oil.

Another embodiment comprises cyclosporin A, Pluronic F68®, and Sunflower oil.

Another embodiment comprises cyclosporin A, Pluronic F68LF®, and Sunflower oil.

Another embodiment comprises cyclosporin A, Pluronic F77®, and Sunflower oil.

Another embodiment comprises cyclosporin A, Pluronic F87®, and Sunflower oil.

Another embodiment comprises cyclosporin A, Pluronic F88®, and Sunflower oil.

Another embodiment comprises cyclosporin A, Pluronic F98®, and Sunflower oil.

Another embodiment comprises cyclosporin A, Pluronic L10®, and Sunflower oil.

Another embodiment comprises cyclosporin A, Pluronic L101®, and Sunflower oil.

Another embodiment comprises cyclosporin A, Pluronic L121®, and Sunflower oil.

Another embodiment comprises cyclosporin A, Pluronic L31®, and Sunflower oil.

Another embodiment comprises cyclosporin A, Pluronic L35®, and Sunflower oil.

Another embodiment comprises cyclosporin A, Pluronic L43®, and Sunflower oil.

Another embodiment comprises cyclosporin A, Pluronic L44®, and Sunflower oil.

Another embodiment comprises cyclosporin A, Pluronic L61®, and Sunflower oil.

Another embodiment comprises cyclosporin A, Pluronic L62®, and Sunflower oil.

Another embodiment comprises cyclosporin A, Pluronic L62D®, and Sunflower oil.

Another embodiment comprises cyclosporin A, Pluronic L62LF®, and Sunflower oil.

Another embodiment comprises cyclosporin A, Pluronic L64®, and Sunflower oil.

Another embodiment comprises cyclosporin A, Pluronic L81®, and Sunflower oil.

Another embodiment comprises cyclosporin A, Pluronic L92®, and Sunflower oil.

Another embodiment comprises cyclosporin A, Pluronic P103®, and Sunflower oil.

Another embodiment comprises cyclosporin A, Pluronic P104®, and Sunflower oil.

Another embodiment comprises cyclosporin A, Pluronic P105®, and Sunflower oil.

Another embodiment comprises cyclosporin A, Pluronic P123®, and Sunflower oil.

Another embodiment comprises cyclosporin A, Pluronic P65®, and Sunflower oil.

Another embodiment comprises cyclosporin A, Pluronic P84®, and Sunflower oil.

Another embodiment comprises cyclosporin A, Pluronic P85®, and Sunflower oil.

Another embodiment comprises cyclosporin A, Polyaldo 10-2-P®, and Sunflower oil.

Another embodiment comprises cyclosporin A, Polyaldo DGD®, and Sunflower oil.

Another embodiment comprises cyclosporin A, Polyaldo HGDS®, and Sunflower oil.

Another embodiment comprises cyclosporin A, Polyaldo TGMS®, and Sunflower oil.

Another embodiment comprises cyclosporin A, Protegin V®, and Sunflower oil.

Another embodiment comprises cyclosporin A, Protegin W®, and Sunflower oil.

Another embodiment comprises cyclosporin A, Protegin WX®, and Sunflower oil.

Another embodiment comprises cyclosporin A, Protegin XV®, and Sunflower oil.

Another embodiment comprises cyclosporin A, Rhodasurf BC-420®, and Sunflower oil.

Another embodiment comprises cyclosporin A, Rhodasurf BC-610®, and Sunflower oil.

Another embodiment comprises cyclosporin A, Rhodasurf BC-630®, and Sunflower oil.

Another embodiment comprises cyclosporin A, Rhodasurf BC-720®, and Sunflower oil.

Another embodiment comprises cyclosporin A, Rhodasurf BC-840®, and Sunflower oil.

Another embodiment comprises cyclosporin A, Rhodasurf L 1®, and Sunflower oil.

Another embodiment comprises cyclosporin A, Rhodasurf L 12®, and Sunflower oil.

Another embodiment comprises cyclosporin A, Rhodasurf L 2®, and Sunflower oil.

Another embodiment comprises cyclosporin A, Rhodasurf L 20®, and Sunflower oil.

Another embodiment comprises cyclosporin A, Rhodasurf L 3®, and Sunflower oil.

Another embodiment comprises cyclosporin A, Rhodasurf L 30®, and Sunflower oil.

Another embodiment comprises cyclosporin A, Rhodasurf L 4®, and Sunflower oil.

Another embodiment comprises cyclosporin A, Rhodasurf L 50®, and Sunflower oil.

Another embodiment comprises cyclosporin A, Rhodasurf L 7/90®, and Sunflower oil.

Another embodiment comprises cyclosporin A, Rhodasurf LA-3®, and Sunflower oil.

Another embodiment comprises cyclosporin A, Rhodasurf LA-7®, and Sunflower oil.

Another embodiment comprises cyclosporin A, Rhodasurf LAN-23®, and Sunflower oil.

Another embodiment comprises cyclosporin A, Rhodasurf ON-870®, and Sunflower oil.

Another embodiment comprises cyclosporin A, Rhodasurf ON-877®, and Sunflower oil.

Another embodiment comprises cyclosporin A, Rhodasurf TDA-8/5®, and Sunflower oil.

Another embodiment comprises cyclosporin A, Ritapro 100®, and Sunflower oil.

Another embodiment comprises cyclosporin A, Ritapro 165®, and Sunflower oil.

Another embodiment comprises cyclosporin A, Ritapro 200®, and Sunflower oil.

Another embodiment comprises cyclosporin A, Ritapro 300®, and Sunflower oil.

Another embodiment comprises cyclosporin A, Ritoleth 10®, and Sunflower oil.

Another embodiment comprises cyclosporin A, Ritoleth 2®, and Sunflower oil.

Another embodiment comprises cyclosporin A, Ritoleth 20®, and Sunflower oil.

Another embodiment comprises cyclosporin A, Ritoleth 5®, and Sunflower oil.

Another embodiment comprises cyclosporin A, Santone 10-10-0®, and Sunflower oil.

Another embodiment comprises cyclosporin A, Santone 3-1-S XTR®, and Sunflower oil.

Another embodiment comprises cyclosporin A, Santone 3-1-SH®and Sunflower oil.

Another embodiment comprises cyclosporin A, Santone 8-1-O®, and Sunflower oil.

Another embodiment comprises cyclosporin A, Solulan 16®, and Sunflower oil.

Another embodiment comprises cyclosporin A, Solulan 75®, and Sunflower oil.

Another embodiment comprises cyclosporin A, Solulan C-24®, and Sunflower oil.

Another embodiment comprises cyclosporin A, Solulan L-575®, and Sunflower oil.

Another embodiment comprises cyclosporin A, Standamox CAW®, and Sunflower oil.

Another embodiment comprises cyclosporin A, Surfine AZI-A®, and Sunflower oil.

Another embodiment comprises cyclosporin A, Surfonic CO-15®, and Sunflower oil.

Another embodiment comprises cyclosporin A, Surfonic CO-25®, and Sunflower oil.

Another embodiment comprises cyclosporin A, Surfonic CO-30®, and Sunflower oil.

Another embodiment comprises cyclosporin A, Surfonic CO-36®, and Sunflower oil.

Another embodiment comprises cyclosporin A, Surfonic CO-42®, and Sunflower oil.

Another embodiment comprises cyclosporin A, Surfonic E400-MO®, and Sunflower oil.

Another embodiment comprises cyclosporin A, Surfonic L12-3®, and Sunflower oil.

Another embodiment comprises cyclosporin A, Surfonic L12-6®, and Sunflower oil.

Another embodiment comprises cyclosporin A, Surfonic L12-8®, and Sunflower oil.

Another embodiment comprises cyclosporin A, Surfonic L24-12®, and Sunflower oil.

Another embodiment comprises cyclosporin A, Surfonic L24-2®, and Sunflower oil.

Another embodiment comprises cyclosporin A, Surfonic L24-22®, and Sunflower oil.

Another embodiment comprises cyclosporin A, Surfonic L24-3®, and Sunflower oil.

Another embodiment comprises cyclosporin A, Surfonic L24-4®, and Sunflower oil.

Another embodiment comprises cyclosporin A, Surfonic L24-5®, and Sunflower oil.

Another embodiment comprises cyclosporin A, Surfonic L24-7®, and Sunflower oil.

Another embodiment comprises cyclosporin A, Surfonic L24-7.1®, and Sunflower oil.

Another embodiment comprises cyclosporin A, Surfonic L24-9®, and Sunflower oil.

Another embodiment comprises cyclosporin A, Surfonic LF-17®, and Sunflower oil.

Another embodiment comprises cyclosporin A, Surfonic LF-18®, and Sunflower oil.

Another embodiment comprises cyclosporin A, Surfonic LF-37®, and Sunflower oil.

Another embodiment comprises cyclosporin A, Sunfonic LF-41®, and Sunflower oil.

Another embodiment comprises cyclosporin A, Surfonic LF-42®, and Sunflower oil.

Another embodiment comprises cyclosporin A, Surfonic LF-47®, and Sunflower oil.

Another embodiment comprises cyclosporin A, Surfonic LF-68®, and Sunflower oil.

Another embodiment comprises cyclosporin A, Surfonic N-10®, and Sunflower oil.

Another embodiment comprises cyclosporin A, Surfonic N-100®, and Sunflower oil.

Another embodiment comprises cyclosporin A, Surfonic N-102®, and Sunflower oil.

Another embodiment comprises cyclosporin A, Surfonic N-120®, and Sunflower oil.

Another embodiment comprises cyclosporin A, Surfonic N-150®, and Sunflower oil.

Another embodiment comprises cyclosporin A, Surfonic N-200®, and Sunflower oil.

Another embodiment comprises cyclosporin A, Surfonic N-300®, and Sunflower oil.

Another embodiment comprises cyclosporin A, Surfonic N-31.5®, and Sunflower oil.

Another embodiment comprises cyclosporin A, Surfonic N-40®, and Sunflower oil.

Another embodiment comprises cyclosporin A, Surfonic N-400®, and Sunflower oil.

Another embodiment comprises cyclosporin A, Surfonic N-60®, and Sunflower oil.

Another embodiment comprises cyclosporin A, Surfonic N-85®, and Sunflower oil.

Another embodiment comprises cyclosporin A, Surfonic N-95®, and Sunflower oil.

Another embodiment comprises cyclosporin A, Surfonic NB-18®, and Sunflower oil.

Another embodiment comprises cyclosporin A, Surfonic NB-23®, and Sunflower oil.

Another embodiment comprises cyclosporin A, Surfonic NB-25®, and Sunflower oil.

Another embodiment comprises cyclosporin A, Surfonic NB-307®, and Sunflower oil.

Another embodiment comprises cyclosporin A, Surfonic NB-31®, and Sunflower oil.

Another embodiment comprises cyclosporin A, Surfonic NB-41®, and Sunflower oil.

Another embodiment comprises cyclosporin A, Surfonic NB-43®, and Sunflower oil.

Another embodiment comprises cyclosporin A, Surfonic OP-100®, and Sunflower oil.

Another embodiment comprises cyclosporin A, Surfonic OP-120®, and Sunflower oil.

Another embodiment comprises cyclosporin A, Surfonic OP-15®, and Sunflower oil.

Another embodiment comprises cyclosporin A, Surfonic OP-35®, and Sunflower oil.

Another embodiment comprises cyclosporin A, Surfonic OP-70®, and Sunflower oil.

Another embodiment comprises cyclosporin A, Surfonic P1200 , and Sunflower oil.

Another embodiment comprises cyclosporin A, Surfonic P3®, and Sunflower oil.

Another embodiment comprises cyclosporin A, Surfonic P5®, and Sunflower oil.

Another embodiment comprises cyclosporin A, Surfonic P6®, and Sunflower oil.

Another embodiment comprises cyclosporin A, Surfonic POA-17R2®, and Sunflower oil.

Another embodiment comprises cyclosporin A, Surfonic POA-25R2®, and Sunflower oil.

Another embodiment comprises cyclosporin A, Surfonic TDA-11®, and Sunflower oil.

Another embodiment comprises cyclosporin A, Surfonic TDA-3B®, and Sunflower oil.

Another embodiment comprises cyclosporin A, Surfonic TDA-6®, and Sunflower oil.

Another embodiment comprises cyclosporin A, Surfonic TDA-6/88®, and Sunflower oil.

Another embodiment comprises cyclosporin A, Surfonic TDA-8®, and Sunflower oil.

Another embodiment comprises cyclosporin A, Surfonic TDA-8/90®, and Sunflower oil.

Another embodiment comprises cyclosporin A, Surfonic TDA-9®, and Sunflower oil.

Another embodiment comprises cyclosporin A, Surfonic TX-CCN®, and Sunflower oil.

Another embodiment comprises cyclosporin A, Tagat CH 40®, and Sunflower oil.

Another embodiment comprises cyclosporin A, Tagat CH 60®, and Sunflower oil.

Another embodiment comprises cyclosporin A, Tagat L 2®, and Sunflower oil.

Another embodiment comprises cyclosporin A, Tagat S®, and Sunflower oil.

Another embodiment comprises cyclosporin A, Tagat S 2®, and Sunflower oil.

Another embodiment comprises cyclosporin A, Tally 100 Plus®, and Sunflower oil.

Another embodiment comprises cyclosporin A, T-Det A106®, and Sunflower oil.

Another embodiment comprises cyclosporin A, T-Det A109®, and Sunflower oil.

Another embodiment comprises cyclosporin A, T-Det A136®, and Sunflower oil.

Another embodiment comprises cyclosporin A, T-Det A139®, and Sunflower oil.

Another embodiment comprises cyclosporin A, T-Det A2412®, and Sunflower oil.

Another embodiment comprises cyclosporin A, T-Det A243®, and Sunflower oil.

Another embodiment comprises cyclosporin A, T-Det A247®, and Sunflower oil.

Another embodiment comprises cyclosporin A, T-Det A249®, and Sunflower oil.

Another embodiment comprises cyclosporin A, T-Det A467®, and Sunflower oil.

Another embodiment comprises cyclosporin A, T-Det COE®, and Sunflower oil.

Another embodiment comprises cyclosporin A, T-Det DD-10®, and Sunflower oil.

Another embodiment comprises cyclosporin A, T-Det DD-5®, and Sunflower oil.

Another embodiment comprises cyclosporin A, T-Det DD-7®, and Sunflower oil.

Another embodiment comprises cyclosporin A, T-Det EPO-104®, and Sunflower oil.

Another embodiment comprises cyclosporin A, T-Det EPO-61®, and Sunflower oil.

Another embodiment comprises cyclosporin A, T-Det EPO-62®, and Sunflower oil.

Another embodiment comprises cyclosporin A, T-Det EPO-64®, and Sunflower oil.

Another embodiment comprises cyclosporin A, T-Det N-1.5®, and Sunflower oil.

Another embodiment comprises cyclosporin A, T-Det N-10.5®, and Sunflower oil.

Another embodiment comprises cyclosporin A, T-Det N-100®, and Sunflower oil.

Another embodiment comprises cyclosporin A, T-Det N-1007®, and Sunflower oil.

Another embodiment comprises cyclosporin A, T-Det N-12®, and Sunflower oil.

Another embodiment comprises cyclosporin A, T-Det N-14®, and Sunflower oil.

Another embodiment comprises cyclosporin A, T-Det N-20®, and Sunflower oil.

Another embodiment comprises cyclosporin A, T-Det N-30®, and Sunflower oil.

Another embodiment comprises cyclosporin A, T-Det N-307®, and Sunflower oil.

Another embodiment comprises cyclosporin A, T-Det N-4®, and Sunflower oil.

Another embodiment comprises cyclosporin A, T-Det N-40®, and Sunflower oil.

Another embodiment comprises cyclosporin A, T-Det N-407®, and Sunflower oil.

Another embodiment comprises cyclosporin A, T-Det N-50®, and Sunflower oil.

Another embodiment comprises cyclosporin A, T-Det N-507®, and Sunflower oil.

Another embodiment comprises cyclosporin A, T-Det N-6®, and Sunflower oil.

Another embodiment comprises cyclosporin A, T-Det N-70®, and Sunflower oil.

Another embodiment comprises cyclosporin A, T-Det N-8®, and Sunflower oil.

Another embodiment comprises cyclosporin A, T-Det N-9.5®, and Sunflower oil.

Another embodiment comprises cyclosporin A, T-Det O-12®, and Sunflower oil.

Another embodiment comprises cyclosporin A, T-Det O-165®, and Sunflower oil.

Another embodiment comprises cyclosporin A, T-Det O-307®, and Sunflower oil.

Another embodiment comprises cyclosporin A, T-Det O-40®, and Sunflower oil.

Another embodiment comprises cyclosporin A, T-Det O-407®, and Sunflower oil.

Another embodiment comprises cyclosporin A, T-Det O-6P, and Sunflower oil.

Another embodiment comprises cyclosporin A, T-Det O-8®, and Sunflower oil.

Another embodiment comprises cyclosporin A, T-Det O-9®, and Sunflower oil.

Another embodiment comprises cyclosporin A, Tegin®, and Sunflower oil.

Another embodiment comprises cyclosporin A, Tergitol 15-S-12®, and Sunflower oil.

Another embodiment comprises cyclosporin A, Tergitol 15-S-15®, and Sunflower oil.

Another embodiment comprises cyclosporin A, Tergitol 15-S-20®, and Sunflower oil.

Another embodiment comprises cyclosporin A, Tergitol 15-S-20 (80AQ)®, and Sunflower oil.

Another embodiment comprises cyclosporin A, Tergitol 15-S-3®, and Sunflower oil.

Another embodiment comprises cyclosporin A, Tergitol 15-S-30®, and Sunflower oil.

Another embodiment comprises cyclosporin A, Tergitol 15-S-40®, and Sunflower oil.

Another embodiment comprises cyclosporin A, Tergitol 15-S-40 (70AQ)®, and Sunflower oil.

Another embodiment comprises cyclosporin A, Tergitol 15-S-5®, and Sunflower oil.

Another embodiment comprises cyclosporin A, Tergitol 15-S-7®, and Sunflower oil.

Another embodiment comprises cyclosporin A, Tergitol 15-S-9®, and Sunflower oil.

Another embodiment comprises cyclosporin A, Tergitol L-101®, and Sunflower oil.

Another embodiment comprises cyclosporin A, Tergitol L-61®, and Sunflower oil.

Another embodiment comprises cyclosporin A, Tergitol L-62®, and Sunflower oil.

Another embodiment comprises cyclosporin A, Tergitol L-64®, and Sunflower oil.

Another embodiment comprises cyclosporin A, Tergitol L-81®, and Sunflower oil.

Another embodiment comprises cyclosporin A, Tergitol NP-10®, and Sunflower oil.

Another embodiment comprises cyclosporin A, Tergitol NP-11®, and Sunflower oil.

Another embodiment comprises cyclosporin A, Tergitol NP-12®, and Sunflower oil.

Another embodiment comprises cyclosporin A, Tergitol NP-13®, and Sunflower oil.

Another embodiment comprises cyclosporin A, Tergitol NP-15®, and Sunflower oil.

Another embodiment comprises cyclosporin A, Tergitol NP-30®, and Sunflower oil.

Another embodiment comprises cyclosporin A, Tergitol NP-30 (70 AQ)®, and Sunflower oil.

Another embodiment comprises cyclosporin A, Tergitol NP-4®, and Sunflower oil.

Another embodiment comprises cyclosporin A, Tergitol NP-40®, and Sunflower oil.

Another embodiment comprises cyclosporin A, Tergitol NP-40 (70 AQ)®, and Sunflower oil.

Another embodiment comprises cyclosporin A, Tergitol NP-50 (70 AQ)®, and Sunflower oil.

Another embodiment comprises cyclosporin A, Tergitol NP-55 (70 AQ)®, and Sunflower oil.

Another embodiment comprises cyclosporin A, Tergitol NP-6®, and Sunflower oil.

Another embodiment comprises cyclosporin A, Tergitol NP-7®, and Sunflower oil.

Another embodiment comprises cyclosporin A, Tergitol NP-70 (70 AQ)®, and Sunflower oil.

Another embodiment comprises cyclosporin A, Tergitol NP-8®, and Sunflower oil.

Another embodiment comprises cyclosporin A, Tergitol NP-9®, and Sunflower oil.

Another embodiment comprises cyclosporin A, Tergitol NP-9.5®, and Sunflower oil.

Another embodiment comprises cyclosporin A, Tergitol TMN-10®, and Sunflower oil.

Another embodiment comprises cyclosporin A, Tergitol TMN-3®, and Sunflower oil.

Another embodiment comprises cyclosporin A, Tergitol TMN-6®, and Sunflower oil.

Another embodiment comprises cyclosporin A, Tergitol XD®, and Sunflower oil.

Another embodiment comprises cyclosporin A, Tergitol XH®, and Sunflower oil.

Another embodiment comprises cyclosporin A, Tergitol XJ®, and Sunflower oil.

Another embodiment comprises cyclosporin A, Tetronic 1107®, and Sunflower oil.

Another embodiment comprises cyclosporin A, Tetronic 1307®, and Sunflower oil.

Another embodiment comprises cyclosporin A, Tetronic 150R1®, and Sunflower oil.

Another embodiment comprises cyclosporin A, Tetronic 304®, and Sunflower oil.

Another embodiment comprises cyclosporin A, Tetronic 701®, and Sunflower oil.

Another embodiment comprises cyclosporin A, Tetronic 901®, and Sunflower oil.

Another embodiment comprises cyclosporin A, Tetronic 904®, and Sunflower oil.

Another embodiment comprises cyclosporin A, Tetronic 908®, and Sunflower oil.

Another embodiment comprises cyclosporin A, Tetronic 90R4s, and Sunflower oil.

Another embodiment comprises cyclosporin A, Tomadol 1-3®, and Sunflower oil.

Another embodiment comprises cyclosporin A, Tomadol 1-5®, and Sunflower oil.

Another embodiment comprises cyclosporin A, Tomadol 1-7®, and Sunflower oil.

Another embodiment comprises cyclosporin A, Tomadol 23-1®, and Sunflower oil.

Another embodiment comprises cyclosporin A, Tomadol 23-3®, and Sunflower oil.

Another embodiment comprises cyclosporin A, Tomadol 23-5®, and Sunflower oil.

Another embodiment comprises cyclosporin A, Tomadol 23-6.5®, and Sunflower oil.

Another embodiment comprises cyclosporin A, Tomadol 25-12®, and Sunflower oil.

Another embodiment comprises cyclosporin A, Tomadol 25-3®, and Sunflower oil.

Another embodiment comprises cyclosporin A, Tomadol 25-7®, and Sunflower oil.

Another embodiment comprises cyclosporin A, Tomadol 25-9®, and Sunflower oil.

Another embodiment comprises cyclosporin A, Tomadol 45-13®, and Sunflower oil.

Another embodiment comprises cyclosporin A, Tomadol 45-2.5®, and Sunflower oil.

Another embodiment comprises cyclosporin A, Tomadol 45-7®, and Sunflower oil.

Another embodiment comprises cyclosporin A, Tomadol 91-2.5®, and Sunflower oil.

Another embodiment comprises cyclosporin A, Tomadol 91-6®, and Sunflower oil.

Another embodiment comprises cyclosporin A, Tomadol 91-8®, and Sunflower oil.

Another embodiment comprises cyclosporin A, Triton X-100®, and Sunflower oil.

Another embodiment comprises cyclosporin A, Triton X-102®, and Sunflower oil.

Another embodiment comprises cyclosporin A, Triton X-114®, and Sunflower oil.

Another embodiment comprises cyclosporin A, Triton X-15®, and Sunflower oil.

Another embodiment comprises cyclosporin A, Triton X-165 (70%)®, and Sunflower oil.

Another embodiment comprises cyclosporin A, Triton X-207®, and Sunflower oil.

Another embodiment comprises cyclosporin A, Triton X-305 (70%)®, and Sunflower oil.

Another embodiment comprises cyclosporin A, Triton X-35®, and Sunflower oil.

Another embodiment comprises cyclosporin A, Triton X-405 (70%)®, and Sunflower oil.

Another embodiment comprises cyclosporin A, Triton X-45®, and Sunflower oil.

Another embodiment comprises cyclosporin A, Triton X-705 (70%)®, and Sunflower oil.

Another embodiment comprises cyclosporin A, Volpo S-10®, and Sunflower oil.

Another embodiment comprises cyclosporin A, Volpo S-2®, and Sunflower oil.

Another embodiment comprises cyclosporin A, Volpo S-20®, and Sunflower oil.

Another embodiment comprises cyclosporin A, Volpo-10®, and Sunflower oil.

Another embodiment comprises cyclosporin A, Volpo-20®, and Sunflower oil.

Another embodiment comprises cyclosporin A, Volpo-3®, and Sunflower oil.

Another embodiment comprises cyclosporin A, Volpo-5®, and Sunflower oil.

Another embodiment comprises cyclosporin A, Abex 2515®, and Eucalyptus oil.

Another embodiment comprises cyclosporin A, Abex 2525/40®, and Eucalyptus oil.

Another embodiment comprises cyclosporin A, Abex 2535®, and Eucalyptus oil.

Another embodiment comprises cyclosporin A, Abex 2545®, and Eucalyptus oil.

Another embodiment comprises cyclosporin A, Acconon E®, and Eucalyptus oil.

Another embodiment comprises cyclosporin A, Alfonic 1216CO-9®, and Eucalyptus oil.

Another embodiment comprises cyclosporin A, Alfonic 1412-3®, and Eucalyptus oil.

Another embodiment comprises cyclosporin A, Alfonic TDA-12®, and Eucalyptus oil.

Another embodiment comprises cyclosporin A, Alfonic TDA-3®, and Eucalyptus oil.

Another embodiment comprises cyclosporin A, Alfonic TDA-4®, and Eucalyptus oil.

Another embodiment comprises cyclosporin A, Alfonic TDA-6®, and Eucalyptus oil.

Another embodiment comprises cyclosporin A, Alfonic TDA-7®, and Eucalyptus oil.

Another embodiment comprises cyclosporin A, Alfonic TDA-8®, and Eucalyptus oil.

Another embodiment comprises cyclosporin A, Alfonic TDA-9®, and Eucalyptus oil.

Another embodiment comprises cyclosporin A, Alkamuls 400-DO®, and Eucalyptus oil.

Another embodiment comprises cyclosporin A, Alkamuls 400-MO/E®, and Eucalyptus oil.

Another embodiment comprises cyclosporin A, Ameroxol OE-10®, and Eucalyptus oil.

Another embodiment comprises cyclosporin A, Ameroxol OE-2®, and Eucalyptus oil.

Another embodiment comprises cyclosporin A, Ameroxol OE-20®, and Eucalyptus oil.

Another embodiment comprises cyclosporin A, Armix 176®, and Eucalyptus oil.

Another embodiment comprises cyclosporin A, Armix 180-C®, and Eucalyptus oil.

Another embodiment comprises cyclosporin A, Armix 183®, and Eucalyptus oil.

Another embodiment comprises cyclosporin A, Brij 35®, and Eucalyptus oil.

Another embodiment comprises cyclosporin A, Brij 35 Liquid®, and Eucalyptus oil.

Another embodiment comprises cyclosporin A, Brij 35 SP®, and Eucalyptus oil.

Another embodiment comprises cyclosporin A, Canasol MJ 2109®, and Eucalyptus oil.

Another embodiment comprises cyclosporin A, Canasol MJ 52®, and Eucalyptus oil.

Another embodiment comprises cyclosporin A, Canasol R 3600®, and Eucalyptus oil.

Another embodiment comprises cyclosporin A, Canasol R 3603®, and Eucalyptus oil.

Another embodiment comprises cyclosporin A, Canasol R 4000 H®, and Eucalyptus oil.

Another embodiment comprises cyclosporin A, Cedepal CA-210®, and Eucalyptus oil.

Another embodiment comprises cyclosporin A, Cedepal CA-520®, and Eucalyptus oil.

Another embodiment comprises cyclosporin A, Cedepal CA-630®, and Eucalyptus oil.

Another embodiment comprises cyclosporin A, Cedepal CA-720®, and Eucalyptus oil.

Another embodiment comprises cyclosporin A, Cedepal CA-890®, and Eucalyptus oil.

Another embodiment comprises cyclosporin A, Cedepal CA-897®, and Eucalyptus oil.

Another embodiment comprises cyclosporin A, Cedepal CO-210®, and Eucalyptus oil.

Another embodiment comprises cyclosporin A, Cedepal CO-430®, and Eucalyptus oil.

Another embodiment comprises cyclosporin A, Cedepal CO-530®, and Eucalyptus oil.

Another embodiment comprises cyclosporin A, Cedepal CO-630®, and Eucalyptus oil.

Another embodiment comprises cyclosporin A, Cedepal CO-710®, and Eucalyptus oil.

Another embodiment comprises cyclosporin A, Cedepal CO-730®, and Eucalyptus oil.

Another embodiment comprises cyclosporin A, Cedepal CO-880®, and Eucalyptus oil.

Another embodiment comprises cyclosporin A, Cedepal CO-887®, and Eucalyptus oil.

Another embodiment comprises cyclosporin A, Cedepal CO-890®, and Eucalyptus oil.

Another embodiment comprises cyclosporin A, Cedepal CO-897®, and Eucalyptus oil.

Another embodiment comprises cyclosporin A, Cedepal CO-970®, and Eucalyptus oil.

Another embodiment comprises cyclosporin A, Cedepal CO-977®, and Eucalyptus oil.

Another embodiment comprises cyclosporin A, Cedepal CO-990®, and Eucalyptus oil.

Another embodiment comprises cyclosporin A, Cedepal CO-997®, and Eucalyptus oil.

Another embodiment comprises cyclosporin A, Chemal BP 261®, and Eucalyptus oil.

Another embodiment comprises cyclosporin A, Chemal BP 262®, and Eucalyptus oil.

Another embodiment comprises cyclosporin A, Chemal BP 3172®, and Eucalyptus oil.

Another embodiment comprises cyclosporin A, Chemal BP 3174®, and Eucalyptus oil.

Another embodiment comprises cyclosporin A, Chemal DA-4®, and Eucalyptus oil.

Another embodiment comprises cyclosporin A, Chemal DA-6®, and Eucalyptus oil.

Another embodiment comprises cyclosporin A, Chemal DA-9®, and Eucalyptus oil.

Another embodiment comprises cyclosporin A, Chemal LA-23®, and Eucalyptus oil.

Another embodiment comprises cyclosporin A, Chemal LA-4®, and Eucalyptus oil.

Another embodiment comprises cyclosporin A, Chemal LA-9®, and Eucalyptus oil.

Another embodiment comprises cyclosporin A, Chemal OA-20G®, and Eucalyptus oil.

Another embodiment comprises cyclosporin A, Chemal OA-5®, and Eucalyptus oil.

Another embodiment comprises cyclosporin A, Chemal OA-9®, and Eucalyptus oil.

Another embodiment comprises cyclosporin A, Chemal TDA-12®, and Eucalyptus oil.

Another embodiment comprises cyclosporin A, Chemal TDA-15®, and Eucalyptus oil.

Another embodiment comprises cyclosporin A, Chemal TDA-3®, and Eucalyptus oil.

Another embodiment comprises cyclosporin A, Chemal TDA-6®, and Eucalyptus oil.

Another embodiment comprises cyclosporin A, Chemal TDA-9®, and Eucalyptus oil.

Another embodiment comprises cyclosporin A, Chemax CO-16®, and Eucalyptus oil.

Another embodiment comprises cyclosporin A, Chemax CO-200/50®, and Eucalyptus oil.

Another embodiment comprises cyclosporin A, Chemax CO-25®, and Eucalyptus oil.

Another embodiment comprises cyclosporin A, Chemax CO-30®, and Eucalyptus oil.

Another embodiment comprises cyclosporin A, Chemax CO-36®, and Eucalyptus oil.

Another embodiment comprises cyclosporin A, Chemax CO-40®, and Eucalyptus oil.

Another embodiment comprises cyclosporin A, Chemax CO-5®, and Eucalyptus oil.

Another embodiment comprises cyclosporin A, Chemax CO-8®, and Eucalyptus oil.

Another embodiment comprises cyclosporin A, Chemax DNP-150/50®, and Eucalyptus oil.

Another embodiment comprises cyclosporin A, Chemax DNP-18®, and Eucalyptus oil.

Another embodiment comprises cyclosporin A, Chemax DNP-8®, and Eucalyptus oil.

Another embodiment comprises cyclosporin A, Chemax E 200 ML®, and Eucalyptus oil.

Another embodiment comprises cyclosporin A, Chemax E 200 MO®, and Eucalyptus oil.

Another embodiment comprises cyclosporin A, Chemax E 400 ML®, and Eucalyptus oil.

Another embodiment comprises cyclosporin A, Chemax E 400 MO®, and Eucalyptus oil.

Another embodiment comprises cyclosporin A, Chemax E 600 ML®, and Eucalyptus oil.

Another embodiment comprises cyclosporin A, Chemax E 600 MO®, and Eucalyptus oil.

Another embodiment comprises cyclosporin A, Chemax E1000 MO®, and Eucalyptus oil.

Another embodiment comprises cyclosporin A, Chemax E1000 MS®, and Eucalyptus oil.

Another embodiment comprises cyclosporin A, Chemax E200 MS®, and Eucalyptus oil.

Another embodiment comprises cyclosporin A, Chemax E400 MS®, and Eucalyptus oil.

Another embodiment comprises cyclosporin A, Chemax E600 MS®, and Eucalyptus oil.

Another embodiment comprises cyclosporin A, Chemax HCO-16®, and Eucalyptus oil.

Another embodiment comprises cyclosporin A, Chemax HCO-200/50®, and Eucalyptus oil.

Another embodiment comprises cyclosporin A, Chemax HCO-25®, and Eucalyptus oil.

Another embodiment comprises cyclosporin A, Chemax HCO-5®, and Eucalyptus oil.

Another embodiment comprises cyclosporin A, Chemax TO-16®, and Eucalyptus oil.

Another embodiment comprises cyclosporin A, Chemax TO-8®, and Eucalyptus oil.

Another embodiment comprises cyclosporin A, Colalube 3409®, and Eucalyptus oil.

Another embodiment comprises cyclosporin A, Colalube 3411®, and Eucalyptus oil.

Another embodiment comprises cyclosporin A, Colalube 3413®, and Eucalyptus oil.

Another embodiment comprises cyclosporin A, Colalube 3414®, and Eucalyptus oil.

Another embodiment comprises cyclosporin A, Colalube 3415®, and Eucalyptus oil.

Another embodiment comprises cyclosporin A, Colalube 3416®, and Eucalyptus oil.

Another embodiment comprises cyclosporin A, Colalube 3417®, and Eucalyptus oil.

Another embodiment comprises cyclosporin A, Delonic NPE-100®, and Eucalyptus oil.

Another embodiment comprises cyclosporin A, Delonic NPE-30®, and Eucalyptus oil.

Another embodiment comprises cyclosporin A, Delonic NPE-40®, and Eucalyptus oil.

Another embodiment comprises cyclosporin A, Delonic-OPE-10®, and Eucalyptus oil.

Another embodiment comprises cyclosporin A, Delonic OPE-12®, and Eucalyptus oil.

Another embodiment comprises cyclosporin A, Delonic OPE-30®, and Eucalyptus oil.

Another embodiment comprises cyclosporin A, Delonic OPE-40®, and Eucalyptus oil.

Another embodiment comprises cyclosporin A, Delonic OPE-5®, and Eucalyptus oil.

Another embodiment comprises cyclosporin A, Delonic OPE-7.5®, and Eucalyptus oil.

Another embodiment comprises cyclosporin A, DePeg 16-CO®, and Eucalyptus oil.

Another embodiment comprises cyclosporin A, DePeg 25-CO®, and Eucalyptus oil.

Another embodiment comprises cyclosporin A, DePeg 30-CO®, and Eucalyptus oil.

Another embodiment comprises cyclosporin A, DePeg 40-CO®, and Eucalyptus oil.

Another embodiment comprises cyclosporin A, DePeg 5-CO®, and Eucalyptus oil.

Another embodiment comprises cyclosporin A, DePeg 80-CO®, and Eucalyptus oil.

Another embodiment comprises cyclosporin A, DeSonic S-100®, and Eucalyptus oil.

Another embodiment comprises cyclosporin A, DeSonic S-114®, and Eucalyptus oil.

Another embodiment comprises cyclosporin A, DeSonic S-405®, and Eucalyptus oil.

Another embodiment comprises cyclosporin A, DeSonic S-45®, and Eucalyptus oil.

Another embodiment comprises cyclosporin A, DeSonic TDA-9®, and Eucalyptus oil.

Another embodiment comprises cyclosporin A, DeThox Acid O-14®, and Eucalyptus oil.

Another embodiment comprises cyclosporin A, DeThox Acid O-16®, and Eucalyptus oil.

Another embodiment comprises cyclosporin A, DeThox Acid O-9®, and Eucalyptus oil.

Another embodiment comprises cyclosporin A, DeThox Acid TO-14®, and Eucalyptus oil.

Another embodiment comprises cyclosporin A, DeThox Acid TO-16.5®, and Eucalyptus oil.

Another embodiment comprises cyclosporin A, DeThox Acid TO-8.5®, and Eucalyptus oil.

Another embodiment comprises cyclosporin A, Dur-Em 114®, and Eucalyptus oil.

Another embodiment comprises cyclosporin A, Dur-Em 117®, and Eucalyptus oil.

Another embodiment comprises cyclosporin A, Dur-Em 204®, and Eucalyptus oil.

Another embodiment comprises cyclosporin A, Dur-Em 207®, and Eucalyptus oil.

Another embodiment comprises cyclosporin A, Dur-Em 207E®, and Eucalyptus oil.

Another embodiment comprises cyclosporin A, Emerest 2704®, and Eucalyptus oil.

Another embodiment comprises cyclosporin A, Emerest 2712®, and Eucalyptus oil.

Another embodiment comprises cyclosporin A, Emerest 2715®, and Eucalyptus oil.

Another embodiment comprises cyclosporin A, Ethal EH-2®, and Eucalyptus oil.

Another embodiment comprises cyclosporin A, Ethal EH-5®, and Eucalyptus oil.

Another embodiment comprises cyclosporin A, Ethal OA-23®, and Eucalyptus oil.

Another embodiment comprises cyclosporin A, Ethosperse CA-20®, and Eucalyptus oil.

Another embodiment comprises cyclosporin A, Ethosperse LA-12®, and Eucalyptus oil.

Another embodiment comprises cyclosporin A, Ethosperse LA-23®, and Eucalyptus oil.

Another embodiment comprises cyclosporin A, Ethosperse LA-4®, and Eucalyptus oil.

Another embodiment comprises cyclosporin A, Ethox 3095®, and Eucalyptus oil.

Another embodiment comprises cyclosporin A, Ethox CO-16b, and Eucalyptus oil.

Another embodiment comprises cyclosporin A, Ethox CO-200®, and Eucalyptus oil.

Another embodiment comprises cyclosporin A, Ethox CO-25®, and Eucalyptus oil.

Another embodiment comprises cyclosporin A, Ethox CO-30®, and Eucalyptus oil.

Another embodiment comprises cyclosporin A, Ethox CO-36®, and Eucalyptus oil.

Another embodiment comprises cyclosporin A, Ethox CO-40®, and Eucalyptus oil.

Another embodiment comprises cyclosporin A, Ethox CO-5®, and Eucalyptus oil.

Another embodiment comprises cyclosporin A, Ethox DO-14®, and Eucalyptus oil.

Another embodiment comprises cyclosporin A, Ethox DO-9®, and Eucalyptus oil.

Another embodiment comprises cyclosporin A, Ethox MA-8®, and Eucalyptus oil.

Another embodiment comprises cyclosporin A, Ethox MI-14®, and Eucalyptus oil.

Another embodiment comprises cyclosporin A, Ethox ML-14®, and Eucalyptus oil.

Another embodiment comprises cyclosporin A, Ethox ML-5®, and Eucalyptus oil.

Another embodiment comprises cyclosporin A, Ethox MO-14®, and Eucalyptus oil.

Another embodiment comprises cyclosporin A, Ethox MO-9®, and Eucalyptus oil.

Another embodiment comprises cyclosporin A, Ethox MS-14®, and Eucalyptus oil.

Another embodiment comprises cyclosporin A, Ethox MS-23®, and Eucalyptus oil.

Another embodiment comprises cyclosporin A, Ethox MS-40®, and Eucalyptus oil.

Another embodiment comprises cyclosporin A, Ethox MS-8®, and Eucalyptus oil.

Another embodiment comprises cyclosporin A, Ethox TO-16®, and Eucalyptus oil.

Another embodiment comprises cyclosporin A, Ethox TO-8®, and Eucalyptus oil.

Another embodiment comprises cyclosporin A, Ethylan SN-120®, and Eucalyptus oil.

Another embodiment comprises cyclosporin A, Ethylan SN-70®, and Eucalyptus oil.

Another embodiment comprises cyclosporin A, Ethylan SN-90®, and Eucalyptus oil.

Another embodiment comprises cyclosporin A, Flexricin 13®, and Eucalyptus oil.

Another embodiment comprises cyclosporin A, Flexricin 15®, and Eucalyptus oil.

Another embodiment comprises cyclosporin A, Genapol 1261®, and Eucalyptus oil.

Another embodiment comprises cyclosporin A, Generol 122 E25®, and Eucalyptus oil.

Another embodiment comprises cyclosporin A, Glicepol 160®, and Eucalyptus oil.

Another embodiment comprises cyclosporin A, Glicepol 560®, and Eucalyptus oil.

Another embodiment comprises cyclosporin A, Glicepol GMS 20®, and Eucalyptus oil.

Another embodiment comprises cyclosporin A, Hetoxide BN-13®, and Eucalyptus oil.

Another embodiment comprises cyclosporin A, Hetoxide DNP-4®, and Eucalyptus oil.

Another embodiment comprises cyclosporin A, Hetoxide DNP-9.6®, and Eucalyptus oil.

Another embodiment comprises cyclosporin A, Hetoxide G-26®, and Eucalyptus oil.

Another embodiment comprises cyclosporin A, Hetoxide G-7®, and Eucalyptus oil.

Another embodiment comprises cyclosporin A, Hetoxide GT-20®, and Eucalyptus oil.

Another embodiment comprises cyclosporin A, Hetoxide HC-40®, and Eucalyptus oil.

Another embodiment comprises cyclosporin A, Hetoxol C-24®, and Eucalyptus oil.

Another embodiment comprises cyclosporin A, Hetoxol CA-10®, and Eucalyptus oil.

Another embodiment comprises cyclosporin A, Hetoxol CA-2®, and Eucalyptus oil.

Another embodiment comprises cyclosporin A, Hetoxol CA-20®, and Eucalyptus oil.

Another embodiment comprises cyclosporin A, Hetoxol CAWS®, and Eucalyptus oil.

Another embodiment comprises cyclosporin A, Hetoxol CD-3®, and Eucalyptus oil.

Another embodiment comprises cyclosporin A, Hetoxol CD-4®, and Eucalyptus oil.

Another embodiment comprises cyclosporin A, Hetoxol CS-15®, and Eucalyptus oil.

Another embodiment comprises cyclosporin A, Hetoxol CS-20®, and Eucalyptus oil.

Another embodiment comprises cyclosporin A, Hetoxol CS-20®, and Eucalyptus oil.

Another embodiment comprises cyclosporin A, Hetoxol CS-25®, and Eucalyptus oil.

Another embodiment comprises cyclosporin A, Hetoxol CS-50®, and Eucalyptus oil.

Another embodiment comprises cyclosporin A, Hetoxol CS-9®, and Eucalyptus oil.

Another embodiment comprises cyclosporin A, Hetoxol CSA-15®, and Eucalyptus oil.

Another embodiment comprises cyclosporin A, Hetoxol D®, and Eucalyptus oil.

Another embodiment comprises cyclosporin A, Hetoxol G®, and Eucalyptus oil.

Another embodiment comprises cyclosporin A, Hetoxol J®, and Eucalyptus oil.

Another embodiment comprises cyclosporin A, Hetoxol L®, and Eucalyptus oil.

Another embodiment comprises cyclosporin A, Hetoxol L-23®, and Eucalyptus oil.

Another embodiment comprises cyclosporin A, Hetoxol L-40®, and Eucalyptus oil.

Another embodiment comprises cyclosporin A, Hetoxol L-9®, and Eucalyptus oil.

Another embodiment comprises cyclosporin A, Hetoxol LS-9®, and Eucalyptus oil.

Another embodiment comprises cyclosporin A, Hetoxol M-3®, and Eucalyptus oil.

Another embodiment comprises cyclosporin A, Hetoxol OA-10 Special®, and Eucalyptus oil.

Another embodiment comprises cyclosporin A, Hetoxol OA-20 Special®, and Eucalyptus oil.

Another embodiment comprises cyclosporin A, Hetoxol OA-3 Special®, and Eucalyptus oil.

Another embodiment comprises cyclosporin A, Hetoxol OA-5 Special®, and Eucalyptus oil.

Another embodiment comprises cyclosporin A, Hetoxol OL-10®, and Eucalyptus oil.

Another embodiment comprises cyclosporin A, Hetoxol OL-2®, and Eucalyptus oil.

Another embodiment comprises cyclosporin A, Hetoxol OL-23®, and Eucalyptus oil.

Another embodiment comprises cyclosporin A, Hetoxol OL-4®, and Eucalyptus oil.

Another embodiment comprises cyclosporin A, Hetoxol OL-40®, and Eucalyptus oil.

Another embodiment comprises cyclosporin A, Hetoxol OL-5®, and Eucalyptus oil.

Another embodiment comprises cyclosporin A, Hetoxol PLA®, and Eucalyptus oil.

Another embodiment comprises cyclosporin A, Hetoxol SP-15®, and Eucalyptus oil.

Another embodiment comprises cyclosporin A, Hetoxol STA-10®, and Eucalyptus oil.

Another embodiment comprises cyclosporin A, Hetoxol STA-2®, and Eucalyptus oil.

Another embodiment comprises cyclosporin A, Hetoxol STA-30®, and Eucalyptus oil.

Another embodiment comprises cyclosporin A, Hetoxol TD-12®, and Eucalyptus oil.

Another embodiment comprises cyclosporin A, Hetoxol TD-18®, and Eucalyptus oil.

Another embodiment comprises cyclosporin A, Hetoxol TD-3®, and Eucalyptus oil.

Another embodiment comprises cyclosporin A, Hetoxol TD-6®, and Eucalyptus oil.

Another embodiment comprises cyclosporin A, Hetoxol TD-9®, and Eucalyptus oil.

Another embodiment comprises cyclosporin A, Hetoxol TDEP-15®, and Eucalyptus oil.

Another embodiment comprises cyclosporin A, Hetoxol TDEP-63®, and Eucalyptus oil.

Another embodiment comprises cyclosporin A, Iconol 24-12®, and Eucalyptus oil.

Another embodiment comprises cyclosporin A, Iconol 24-23®, and Eucalyptus oil.

Another embodiment comprises cyclosporin A, Iconol 24-4®, and Eucalyptus oil.

Another embodiment comprises cyclosporin A, Iconol DA-4®, and Eucalyptus oil.

Another embodiment comprises cyclosporin A, Iconol DA-6®, and Eucalyptus oil.

Another embodiment comprises cyclosporin A, Iconol TDA-10®, and Eucalyptus oil.

Another embodiment comprises cyclosporin A, Iconol TDA-3®, and Eucalyptus oil.

Another embodiment comprises cyclosporin A, Iconol TDA-6®, and Eucalyptus oil.

Another embodiment comprises cyclosporin A, Iconol TDA-8-90%®, and Eucalyptus oil.

Another embodiment comprises cyclosporin A, Iconol TDA-9®, and Eucalyptus oil.

Another embodiment comprises cyclosporin A, Igepal CA-210®, and Eucalyptus oil.

Another embodiment comprises cyclosporin A, Igepal CA-407®, and Eucalyptus oil.

Another embodiment comprises cyclosporin A, Igepal CA-420®, and Eucalyptus oil.

Another embodiment comprises cyclosporin A, Igepal CA-520®, and Eucalyptus oil.

Another embodiment comprises cyclosporin A, Igepal CA-620®, and Eucalyptus oil.

Another embodiment comprises cyclosporin A, Igepal CA-630®, and Eucalyptus oil.

Another embodiment comprises cyclosporin A, Igepal CA-720®, and Eucalyptus oil.

Another embodiment comprises cyclosporin A, Igepal CA-877®, and Eucalyptus oil.

Another embodiment comprises cyclosporin A, Igepal CA-887®, and Eucalyptus oil.

Another embodiment comprises cyclosporin A, Igepal CA-897®, and Eucalyptus oil.

Another embodiment comprises cyclosporin A, Igepal CO-210®, and Eucalyptus oil.

Another embodiment comprises cyclosporin A, Igepal CO-430®, and Eucalyptus oil.

Another embodiment comprises cyclosporin A, Igepal CO-520®, and Eucalyptus oil.

Another embodiment comprises cyclosporin A, Igepal CO-530®, and Eucalyptus oil.

Another embodiment comprises cyclosporin A, Igepal CO-610®, and Eucalyptus oil.

Another embodiment comprises cyclosporin A, Igepal CO-630®, and Eucalyptus oil.

Another embodiment comprises cyclosporin A, Igepal CO-630 Special®, and Eucalyptus oil.

Another embodiment comprises cyclosporin A, Igepal CO-660®, and Eucalyptus oil.

Another embodiment comprises cyclosporin A, Igepal CO-710®, and Eucalyptus oil.

Another embodiment comprises cyclosporin A, Igepal CO-720®, and Eucalyptus oil.

Another embodiment comprises cyclosporin A, Igepal CO-730®, and Eucalyptus oil.

Another embodiment comprises cyclosporin A, Igepal CO-738®, and Eucalyptus oil.

Another embodiment comprises cyclosporin A, Igepal CO-850®, and Eucalyptus oil.

Another embodiment comprises cyclosporin A, Igepal CO-880®, and Eucalyptus oil.

Another embodiment comprises cyclosporin A, Igepal CO-887®, and Eucalyptus oil.

Another embodiment comprises cyclosporin A, Igepal CO-890®, and Eucalyptus oil.

Another embodiment comprises cyclosporin A, Igepal CO-897®, and Eucalyptus oil.

Another embodiment comprises cyclosporin A, Igepal CO-970®, and Eucalyptus oil.

Another embodiment comprises cyclosporin A, Igepal CO-977®, and Eucalyptus oil.

Another embodiment comprises cyclosporin A, Igepal CO-987®, and Eucalyptus oil.

Another embodiment comprises cyclosporin A, Igepal CO-990FLK®, and Eucalyptus oil.

Another embodiment comprises cyclosporin A, Igepal CO-997®, and Eucalyptus oil.

Another embodiment comprises cyclosporin A, Igepal DM-430®, and Eucalyptus oil.

Another embodiment comprises cyclosporin A, Igepal DM-530®, and Eucalyptus oil.

Another embodiment comprises cyclosporin A, Igepal DM-710®, and Eucalyptus oil.

Another embodiment comprises cyclosporin A, Igepal DM-730®, and Eucalyptus oil.

Another embodiment comprises cyclosporin A, Igepal DM-880®, and Eucalyptus oil.

Another embodiment comprises cyclosporin A, Igepal DM-970 FLK®, and Eucalyptus oil.

Another embodiment comprises cyclosporin A, Igepal OD-410®, and Eucalyptus oil.

Another embodiment comprises cyclosporin A, Igepal RC-520®, and Eucalyptus oil.

Another embodiment comprises cyclosporin A, Igepal RC-620®, and Eucalyptus oil.

Another embodiment comprises cyclosporin A, Igepal RC-630®, and Eucalyptus oil.

Another embodiment comprises cyclosporin A, Lexemul CS-20®, and Eucalyptus oil.

Another embodiment comprises cyclosporin A, Lipo GMS 450®, and Eucalyptus oil.

Another embodiment comprises cyclosporin A, Lipo Polyglycol 1000®, and Eucalyptus oil.

Another embodiment comprises cyclosporin A, Lipo Polyglycol 200®, and Eucalyptus oil.

Another embodiment comprises cyclosporin A, Lipo Polyglycol 300®, and Eucalyptus oil.

Another embodiment comprises cyclosporin A, Lipo Polyglycol 3350®, and Eucalyptus oil.

Another embodiment comprises cyclosporin A, Lipo Polyglycol 400®, and Eucalyptus oil.

Another embodiment comprises cyclosporin A, Lipo Polyglycol 600®, and Eucalyptus oil.

Another embodiment comprises cyclosporin A, Lipocol C-10®, and Eucalyptus oil.

Another embodiment comprises cyclosporin A, Lipocol C-2®, and Eucalyptus oil.

Another embodiment comprises cyclosporin A, Lipocol C-20®, and Eucalyptus oil.

Another embodiment comprises cyclosporin A, Lipocol L-12®, and Eucalyptus oil.

Another embodiment comprises cyclosporin A, Lipocol L-23®, and Eucalyptus oil.

Another embodiment comprises cyclosporin A, Lipocol L-4®, and Eucalyptus oil.

Another embodiment comprises cyclosporin A, Lipocol O-10®, and Eucalyptus oil.

Another embodiment comprises cyclosporin A, Lipocol O-2®, and Eucalyptus oil.

Another embodiment comprises cyclosporin A, Lipocol O-20®, and Eucalyptus oil.

Another embodiment comprises cyclosporin A, Lipocol O-5®, and Eucalyptus oil.

Another embodiment comprises cyclosporin A, Lipocol S-10®, and Eucalyptus oil.

Another embodiment comprises cyclosporin A, Lipocol S-2®, and Eucalyptus oil.

Another embodiment comprises cyclosporin A, Lipocol S-20®, and Eucalyptus oil.

Another embodiment comprises cyclosporin A, Lipocol SC-15®, and Eucalyptus oil.

Another embodiment comprises cyclosporin A, Lipocol SC-20®, and Eucalyptus oil.

Another embodiment comprises cyclosporin A, Lipocol SC-4®, and Eucalyptus oil.

Another embodiment comprises cyclosporin A, Lipocol TD-12®, and Eucalyptus oil.

Another embodiment comprises cyclosporin A, Lumulse 40-O K®, and Eucalyptus oil.

Another embodiment comprises cyclosporin A, Lumulse 42-O K®, and Eucalyptus oil.

Another embodiment comprises cyclosporin A, Lumulse GMT-40®, and Eucalyptus oil.

Another embodiment comprises cyclosporin A, Lumulse PEG 1450®, and Eucalyptus oil.

Another embodiment comprises cyclosporin A, Lumulse PEG 1450 NF®, and Eucalyptus oil.

Another embodiment comprises cyclosporin A, Lumulse PEG 8000®, and Eucalyptus oil.

Another embodiment comprises cyclosporin A, Lumulse PGO®, and Eucalyptus oil.

Another embodiment comprises cyclosporin A, Lumulse POE (2) Oleyl Amine®, and Eucalyptus oil.

Another embodiment comprises cyclosporin A, Magrabar PGO-104®, and Eucalyptus oil.

Another embodiment comprises cyclosporin A, Mapeg 200 ML®, and Eucalyptus oil.

Another embodiment comprises cyclosporin A, Mapeg 400 MO®, and Eucalyptus oil.

Another embodiment comprises cyclosporin A, Mapeg 400 MOT®, and Eucalyptus oil.

Another embodiment comprises cyclosporin A, Mapeg 400DO®, and Eucalyptus oil.

Another embodiment comprises cyclosporin A, Mapeg 400DOT®, and Eucalyptus oil.

Another embodiment comprises cyclosporin A, Mapeg 600DOT®, and Eucalyptus oil.

Another embodiment comprises cyclosporin A, Marlox B 24/80®, and Eucalyptus oil.

Another embodiment comprises cyclosporin A, Marlox FK 86®, and Eucalyptus oil.

Another embodiment comprises cyclosporin A, Marlox MO 154®, and Eucalyptus oil.

Another embodiment comprises cyclosporin A, Mazox CDA CG®, and Eucalyptus oil.

Another embodiment comprises cyclosporin A, Monoglycerides Glyceryl Monestearate®, and Eucalyptus oil.

Another embodiment comprises cyclosporin A, Myrj 45®, and Eucalyptus oil.

Another embodiment comprises cyclosporin A, Myrj 52®, and Eucalyptus oil.

Another embodiment comprises cyclosporin A, Myrj 52S®, and Eucalyptus oil.

Another embodiment comprises cyclosporin A, Myrj 53®, and Eucalyptus oil.

Another embodiment comprises cyclosporin A, Myrj 59®, and Eucalyptus oil.

Another embodiment comprises cyclosporin A, Norfox NP-12®, and Eucalyptus oil.

Another embodiment comprises cyclosporin A, Norfox NP-4®, and Eucalyptus oil.

Another embodiment comprises cyclosporin A, Norlox NP-6®, and Eucalyptus oil.

Another embodiment comprises cyclosporin A, Norfox NP-887®, and Eucalyptus oil.

Another embodiment comprises cyclosporin A, Norfox NP-9®, and Eucalyptus oil.

Another embodiment comprises cyclosporin A, Norfox NP-977®, and Eucalyptus oil.

Another embodiment comprises cyclosporin A, Norfox OP-100®, and Eucalyptus oil.

Another embodiment comprises cyclosporin A, Norfox OP-102®, and Eucalyptus oil.

Another embodiment comprises cyclosporin A, Norfox OP-114®, and Eucalyptus oil.

Another embodiment comprises cyclosporin A, Norfox OP-45®, and Eucalyptus oil.

Another embodiment comprises cyclosporin A, Pegosperse 100-L®, and Eucalyptus oil.

Another embodiment comprises cyclosporin A, Pegosperse 100-O®, and Eucalyptus oil.

Another embodiment comprises cyclosporin A, Pegosperse 100-S®, and Eucalyptus oil.

Another embodiment comprises cyclosporin A, Pegosperse 1500-MS®, and Eucalyptus oil.

Another embodiment comprises cyclosporin A, Pegosperse 1750-MS®, and Eucalyptus oil.

Another embodiment comprises cyclosporin A, Pegosperse 200 DL®, and Eucalyptus oil.

Another embodiment comprises cyclosporin A, Pegosperse 200-ML®, and Eucalyptus oil.

Another embodiment comprises cyclosporin A, Pegosperse 400-DL®, and Eucalyptus oil.

Another embodiment comprises cyclosporin A, Pegosperse 400-DO®, and Eucalyptus oil.

Another embodiment comprises cyclosporin A, Pegosperse 400-DS®, and Eucalyptus oil.

Another embodiment comprises cyclosporin A, Pegosperse 400-ML®, and Eucalyptus oil.

Another embodiment comprises cyclosporin A, Pegosperse 400-MO®, and Eucalyptus oil.

Another embodiment comprises cyclosporin A, Pegosperse 400-MOT®, and Eucalyptus oil.

Another embodiment comprises cyclosporin A, Pegosperse 400-MS®, and Eucalyptus oil.

Another embodiment comprises cyclosporin A, Pegosperse 50-DS®, and Eucalyptus oil.

Another embodiment comprises cyclosporin A, Pegosperse 50-MS®, and Eucalyptus oil.

Another embodiment comprises cyclosporin A, Pegosperse 600-ML®, and Eucalyptus oil.

Another embodiment comprises cyclosporin A, Pegosperse 600-MS®, and Eucalyptus oil.

Another embodiment comprises cyclosporin A, Plurafac A-38®, and Eucalyptus oil.

Another embodiment comprises cyclosporin A, Plurafac B-25-50®, and Eucalyptus oil.

Another embodiment comprises cyclosporin A, Plurafac B-26®, and Eucalyptus oil.

Another embodiment comprises cyclosporin A, Plurafac D-25®, and Eucalyptus oil.

Another embodiment comprises cyclosporin A, Plurafac LF 131®, and Eucalyptus oil.

Another embodiment comprises cyclosporin A, Plurafac LF 4030®, and Eucalyptus oil.

Another embodiment comprises cyclosporin A, Plurafac LF 7000®, and Eucalyptus oil.

Another embodiment comprises cyclosporin A, Plurafac RA-20®, and Eucalyptus oil.

Another embodiment comprises cyclosporin A, Plurafac RA-30®, and Eucalyptus oil.

Another embodiment comprises cyclosporin A, Plurafac RA-40®, and Eucalyptus oil.

Another embodiment comprises cyclosporin A, Pluronic 10R5®, and Eucalyptus oil.

Another embodiment comprises cyclosporin A, Pluronic 17R2®, and Eucalyptus oil.

Another embodiment comprises cyclosporin A, Pluronic 17R4®, and Eucalyptus oil.

Another embodiment comprises cyclosporin A, Pluronic 25R2®, and Eucalyptus oil.

Another embodiment comprises cyclosporin A, Pluronic 25R43®, and Eucalyptus oil.

Another embodiment comprises cyclosporin A, Pluronic 31R1®, and Eucalyptus oil.

Another embodiment comprises cyclosporin A, Pluronic F 108®, and Eucalyptus oil.

Another embodiment comprises cyclosporin A, Pluronic F127®, and Eucalyptus oil.

Another embodiment comprises cyclosporin A, Pluronic F38®, and Eucalyptus oil.

Another embodiment comprises cyclosporin A, Pluronic F68®, and Eucalyptus oil.

Another embodiment comprises cyclosporin A, Pluronic F68LF®, and Eucalyptus oil.

Another embodiment comprises cyclosporin A, Pluronic F77®, and Eucalyptus oil.

Another embodiment comprises cyclosporin A, Pluronic F87®, and Eucalyptus oil.

Another embodiment comprises cyclosporin A, Pluronic F88®, and Eucalyptus oil.

Another embodiment comprises cyclosporin A, Pluronic F98®, and Eucalyptus oil.

Another embodiment comprises cyclosporin A, Pluronic L10®, and Eucalyptus oil.

Another embodiment comprises cyclosporin A, Pluronic L101®, and Eucalyptus oil.

Another embodiment comprises cyclosporin A, Pluronic L121®, and Eucalyptus oil.

Another embodiment comprises cyclosporin A, Pluronic L31®, and Eucalyptus oil.

Another embodiment comprises cyclosporin A, Pluronic L35®, and Eucalyptus oil.

Another embodiment comprises cyclosporin A, Pluronic L43®, and Eucalyptus oil.

Another embodiment comprises cyclosporin A, Pluronic L44®, and Eucalyptus oil.

Another embodiment comprises cyclosporin A, Pluronic L61®, and Eucalyptus oil.

Another embodiment comprises cyclosporin A, Pluronic L62®, and Eucalyptus oil.

Another embodiment comprises cyclosporin A, Pluronic L62D®, and Eucalyptus oil.

Another embodiment comprises cyclosporin A, Pluronic L62LF®, and Eucalyptus oil.

Another embodiment comprises cyclosporin A, Pluronic L64®, and Eucalyptus oil.

Another embodiment comprises cyclosporin A, Pluronic L81®, and Eucalyptus oil.

Another embodiment comprises cyclosporin A, Pluronic L92®, and Eucalyptus oil.

Another embodiment comprises cyclosporin A, Pluronic P103®, and Eucalyptus oil.

Another embodiment comprises cyclosporin A, Pluronic P104®, and Eucalyptus oil.

Another embodiment comprises cyclosporin A, Pluronic P105®, and Eucalyptus oil.

Another embodiment comprises cyclosporin A, Pluronic P123®, and Eucalyptus oil.

Another embodiment comprises cyclosporin A, Pluronic P65®, and Eucalyptus oil.

Another embodiment comprises cyclosporin A, Pluronic P84®, and Eucalyptus oil.

Another embodiment comprises cyclosporin A, Pluronic P85®, and Eucalyptus oil.

Another embodiment comprises cyclosporin A, Polyaldo 10-2-P®, and Eucalyptus oil.

Another embodiment comprises cyclosporin A, Polyaldo DGDO®, and Eucalyptus oil.

Another embodiment comprises cyclosporin A, Polyaldo HGDS®, and Eucalyptus oil.

Another embodiment comprises cyclosporin A, Polyaldo TGMS®, and Eucalyptus oil.

Another embodiment comprises cyclosporin A, Protegin V®, and Eucalyptus oil.

Another embodiment comprises cyclosporin A, Protegin W®, and Eucalyptus oil.

Another embodiment comprises cyclosporin A, Protegin WX®, and Eucalyptus oil.

Another embodiment comprises cyclosporin A, Protegin XV®, and Eucalyptus oil.

Another embodiment comprises cyclosporin A, Rhodasurf BC-420®, and Eucalyptus oil.

Another embodiment comprises cyclosporin A, Rhodasurf BC-610®, and Eucalyptus oil.

Another embodiment comprises cyclosporin A, Rhodasurf BC-630®, and Eucalyptus oil.

Another embodiment comprises cyclosporin A, Rhodasurf BC-720®, and Eucalyptus oil.

Another embodiment comprises cyclosporin A, Rhodasurf BC-840®, and Eucalyptus oil.

Another embodiment comprises cyclosporin A, Rhodasurf L 1®, and Eucalyptus oil.

Another embodiment comprises cyclosporin A, Rhodasurf L 12®, and Eucalyptus oil.

Another embodiment comprises cyclosporin A, Rhodasurf L 2®, and Eucalyptus oil.

Another embodiment comprises cyclosporin A, Rhodasurf L 20®, and Eucalyptus oil.

Another embodiment comprises cyclosporin A, Rhodasurf L 3®, and Eucalyptus oil.

Another embodiment comprises cyclosporin A, Rhodasurf L 30®, and Eucalyptus oil.

Another embodiment comprises cyclosporin A, Rhodasurf L 4®, and Eucalyptus oil.

Another embodiment comprises cyclosporin A, Rhodasurf L 50®, and Eucalyptus oil.

Another embodiment comprises cyclosporin A, Rhodasurf L 7/90®, and Eucalyptus oil.

Another embodiment comprises cyclosporin A, Rhodasurf LA-3®, and Eucalyptus oil.

Another embodiment comprises cyclosporin A, Rhodasurf LA-7®, and Eucalyptus oil.

Another embodiment comprises cyclosporin A, Rhodasurf LAN-23®, and Eucalyptus oil.

Another embodiment comprises cyclosporin A, Rhodasurf ON-870®, and Eucalyptus oil.

Another embodiment comprises cyclosporin A, Rhodasurf ON-877®, and Eucalyptus oil.

Another embodiment comprises cyclosporin A, Rhodasurf TDA-8/5®, and Eucalyptus oil.

Another embodiment comprises cyclosporin A, Ritapro 100®, and Eucalyptus oil.

Another embodiment comprises cyclosporin A, Ritapro 165®, and Eucalyptus oil.

Another embodiment comprises cyclosporin A, Ritapro 200®, and Eucalyptus oil.

Another embodiment comprises cyclosporin A, Ritapro 300®, and Eucalyptus oil.

Another embodiment comprises cyclosporin A, Ritoleth 10®, and Eucalyptus oil.

Another embodiment comprises cyclosporin A, Ritoleth 2®, and Eucalyptus oil.

Another embodiment comprises cyclosporin A, Ritoleth 20®, and Eucalyptus oil.

Another embodiment comprises cyclosporin A, Ritoleth 5®, and Eucalyptus oil.

Another embodiment comprises cyclosporin A, Santone 10-10-0®, and Eucalyptus oil.

Another embodiment comprises cyclosporin A, Santone 3-1-S XTR®, and Eucalyptus oil.

Another embodiment comprises cyclosporin A, Santone 3-1 -SH®and Eucalyptus oil.

Another embodiment comprises cyclosporin A, Santone 8-1-O®, and Eucalyptus oil.

Another embodiment comprises cyclosporin A, Solulan 16®, and Eucalyptus oil.

Another embodiment comprises cyclosporin A, Solulan 75®, and Eucalyptus oil.

Another embodiment comprises cyclosporin A, Solulan C-24®, and Eucalyptus oil.

Another embodiment comprises cyclosporin A, Solulan L-575®, and Eucalyptus oil.

Another embodiment comprises cyclosporin A, Standamox CAW®, and Eucalyptus oil.

Another embodiment comprises cyclosporin A, Surfine AZI-A®, and Eucalyptus oil.

Another embodiment comprises cyclosporin A, Surfonic CO-15®, and Eucalyptus oil.

Another embodiment comprises cyclosporin A, Surfonic CO-25®, and Eucalyptus oil.

Another embodiment comprises cyclosporin A, Surfonic CO-30®, and Eucalyptus oil.

Another embodiment comprises cyclosporin A, Surfonic CO-36®, and Eucalyptus oil.

Another embodiment comprises cyclosporin A, Suraonic CO-42®, and Eucalyptus oil.

Another embodiment comprises cyclosporin A, Surfonic E400MO®, and Eucalyptus oil.

Another embodiment comprises cyclosporin A, Surfonic L12-3®, and Eucalyptus oil.

Another embodiment comprises cyclosporin A, Surfonic L12-6®, and Eucalyptus oil.

Another embodiment comprises cyclosporin A, Surfonic L12-8®, and Eucalyptus oil.

Another embodiment comprises cyclosporin A, Surfonic L24-12®, and Eucalyptus oil.

Another embodiment comprises cyclosporin A, Surfonic L24-2®, and Eucalyptus oil.

Another embodiment comprises cyclosporin A, Surfonic L24-22®, and Eucalyptus oil.

Another embodiment comprises cyclosporin A, Surfonic L24-3®, and Eucalyptus oil.

Another embodiment comprises cyclosporin A, Surfonic L24-4®, and Eucalyptus oil.

Another embodiment comprises cyclosporin A, Surfonic L24-5®, and Eucalyptus oil.

Another embodiment comprises cyclosporin A, Surfonic L24-7®, and Eucalyptus oil.

Another embodiment comprises cyclosporin A, Surfonic L24-7.1®, and Eucalyptus oil.

Another embodiment comprises cyclosporin A, Surfonic L24-9®, and Eucalyptus oil.

Another embodiment comprises cyclosporin A, Surfonic LF-17®, and Eucalyptus oil.

Another embodiment comprises cyclosporin A, Surfonic LF-18®, and Eucalyptus oil.

Another embodiment comprises cyclosporin A, Surfonic LF-37®, and Eucalyptus oil.

Another embodiment comprises cyclosporin A, Surfonic LF-41®, and Eucalyptus oil.

Another embodiment comprises cyclosporin A, Surfonic LF-42®, and Eucalyptus oil.

Another embodiment comprises cyclosporin A, Surfonic LF-47®, and Eucalyptus oil.

Another embodiment comprises cyclosporin A, Surfonic LF-68®, and Eucalyptus oil.

Another embodiment comprises cyclosporin A, Surfonic N-10®, and Eucalyptus oil.

Another embodiment comprises cyclosporin A, Surfonic N-100®, and Eucalyptus oil.

Another embodiment comprises cyclosporin A, Surfonic N-102®, and Eucalyptus oil.

Another embodiment comprises cyclosporin A, Surfonic N-120®, and Eucalyptus oil.

Another embodiment comprises cyclosporin A, Surfonic N-150®, and Eucalyptus oil.

Another embodiment comprises cyclosporin A, Surfonic N-200®, and Eucalyptus oil.

Another embodiment comprises cyclosporin A, Surfonic N-300®, and Eucalyptus oil.

Another embodiment comprises cyclosporin A, Surfonic N-31.5®, and Eucalyptus oil.

Another embodiment comprises cyclosporin A, Surfonic N-40®, and Eucalyptus oil.

Another embodiment comprises cyclosporin A, Surfonic N-400®, and Eucalyptus oil.

Another embodiment comprises cyclosporin A, Surfonic N-60®, and Eucalyptus oil.

Another embodiment comprises cyclosporin A, Surfonic N-85®, and Eucalyptus oil.

Another embodiment comprises cyclosporin A, Surfonic N-95®, and Eucalyptus oil.

Another embodiment comprises cyclosporin A, Surfonic NB-18®, and Eucalyptus oil.

Another embodiment comprises cyclosporin A, Surfonic NB-23®, and Eucalyptus oil.

Another embodiment comprises cyclosporin A, Surfonic NB-25®, and Eucalyptus oil.

Another embodiment comprises cyclosporin A, Surfonic NB-307®, and Eucalyptus oil.

Another embodiment comprises cyclosporin A, Surfonic NB-31®, and Eucalyptus oil.

Another embodiment comprises cyclosporin A, Surfonic NB-41®, and Eucalyptus oil.

Another embodiment comprises cyclosporin A, Surfonic NB-43®, and Eucalyptus oil.

Another embodiment comprises cyclosporin A, Surfonic OP-100®, and Eucalyptus oil.

Another embodiment comprises cyclosporin A, Surfonic OP-120®, and Eucalyptus oil.

Another embodiment comprises cyclosporin A, Surfonic OP-15®, and Eucalyptus oil.

Another embodiment comprises cyclosporin A, Surfonic OP-35®, and Eucalyptus oil.

Another embodiment comprises cyclosporin A, Surfonic OP-70®, and Eucalyptus oil.

Another embodiment comprises cyclosporin A, Surfonic P1®, and Eucalyptus oil.

Another embodiment comprises cyclosporin A, Surfonic P3®, and Eucalyptus oil.

Another embodiment comprises cyclosporin A, Surfonic P5®, and Eucalyptus oil.

Another embodiment comprises cyclosporin A, Surfonic P6®, and Eucalyptus oil.

Another embodiment comprises cyclosporin A, Surfonic POA-17R2®, and Eucalyptus oil.

Another embodiment comprises cyclosporin A, Surfonic POA-25R2®, and Eucalyptus oil.

Another embodiment comprises cyclosporin A, Surfonic TDA-11®, and Eucalyptus oil.

Another embodiment comprises cyclosporin A, Surfonic TDA-3B®, and Eucalyptus oil.

Another embodiment comprises cyclosporin A, Surfonic TDA-6®, and Eucalyptus oil.

Another embodiment comprises cyclosporin A, Surfonic TDA-6/88®, and Eucalyptus oil.

Another embodiment comprises cyclosporin A, Surfonic TDA-8®, and Eucalyptus oil.

Another embodiment comprises cyclosporin A, Surfonic TDA-8/90®, and Eucalyptus oil.

Another embodiment comprises cyclosporin A, Surfonic TDA-9®, and Eucalyptus oil.

Another embodiment comprises cyclosporin A, Surfonic TX-CCN®, and Eucalyptus oil.

Another embodiment comprises cyclosporin A, Tagat CH 40®, and Eucalyptus oil.

Another embodiment comprises cyclosporin A, Tagat CH 60®, and Eucalyptus oil.

Another embodiment comprises cyclosporin A, Tagat L 2®, and Eucalyptus oil.

Another embodiment comprises cyclosporin A, Tagat S®, and Eucalyptus oil.

Another embodiment comprises cyclosporin A, Tagat S 2®, and Eucalyptus oil.

Another embodiment comprises cyclosporin A, Tally 100 Plus®, and Eucalyptus oil.

Another embodiment comprises cyclosporin A, T-Det A106®, and Eucalyptus oil.

Another embodiment comprises cyclosporin A, T-Det A109®, and Eucalyptus oil.

Another embodiment comprises cyclosporin A, T-Det A136®, and Eucalyptus oil.

Another embodiment comprises cyclosporin A, T-Det A139®, and Eucalyptus oil.

Another embodiment comprises cyclosporin A, T-Det A2412®, and Eucalyptus oil.

Another embodiment comprises cyclosporin A, T-Det A243®, and Eucalyptus oil.

Another embodiment comprises cyclosporin A, T-Det A247®, and Eucalyptus oil.

Another embodiment comprises cyclosporin A, T-Det A249®, and Eucalyptus oil.

Another embodiment comprises cyclosporin A, T-Det A467®, and Eucalyptus oil.

Another embodiment comprises cyclosporin A, T-Det COE®, and Eucalyptus oil.

Another embodiment comprises cyclosporin A, T-Det DD-10®, and Eucalyptus oil.

Another embodiment comprises cyclosporin A, T-Det DD-5®, and Eucalyptus oil.

Another embodiment comprises cyclosporin A, T-Det DD-7®, and Eucalyptus oil.

Another embodiment comprises cyclosporin A, T-Det EPO-104®, and Eucalyptus oil.

Another embodiment comprises cyclosporin A, T-Det EPO-61®, and Eucalyptus oil.

Another embodiment comprises cyclosporin A, T-Det EPO-62®, and Eucalyptus oil.

Another embodiment comprises cyclosporin A, T-Det EPO-64®, and Eucalyptus oil.

Another embodiment comprises cyclosporin A, T-Det N-1.5®, and Eucalyptus oil.

Another embodiment comprises cyclosporin A, T-Det N-10.5®, and Eucalyptus oil.

Another embodiment comprises cyclosporin A, T-Det N-100®, and Eucalyptus oil.

Another embodiment comprises cyclosporin A, T-Det N-1007®, and Eucalyptus oil.

Another embodiment comprises cyclosporin A, T-Det N-12®, and Eucalyptus oil.

Another embodiment comprises cyclosporin A, T-Det N-14®, and Eucalyptus oil.

Another embodiment comprises cyclosporin A, T-Det N-20®, and Eucalyptus oil.

Another embodiment comprises cyclosporin A, T-Det N-30®, and Eucalyptus oil.

Another embodiment comprises cyclosporin A, T-Det N-307®, and Eucalyptus oil.

Another embodiment comprises cyclosporin A, T-Det N-4®, and Eucalyptus oil.

Another embodiment comprises cyclosporin A, T-Det N-40®, and Eucalyptus oil.

Another embodiment comprises cyclosporin A, T-Det N-407®, and Eucalyptus oil.

Another embodiment comprises cyclosporin A, T-Det N-50®, and Eucalyptus oil.

Another embodiment comprises cyclosporin A, T-Det N-507®, and Eucalyptus oil.

Another embodiment comprises cyclosporin A, T-Det N-6®, and Eucalyptus oil.

Another embodiment comprises cyclosporin A, T-Det N-70®, and Eucalyptus oil.

Another embodiment comprises cyclosporin A, T-Det N-8®, and Eucalyptus oil.

Another embodiment comprises cyclosporin A, T-Det N-9.5®, and Eucalyptus oil.

Another embodiment comprises cyclosporin A, T-Det O-12®, and Eucalyptus oil.

Another embodiment comprises cyclosporin A, T-Det O-165®, and Eucalyptus oil.

Another embodiment comprises cyclosporin A, T-Det O-307®, and Eucalyptus oil.

Another embodiment comprises cyclosporin A, T-Det O-40®, and Eucalyptus oil.

Another embodiment comprises cyclosporin A, T-Det O-407®, and Eucalyptus oil.

Another embodiment comprises cyclosporin A, T-Det O-6®, and Eucalyptus oil.

Another embodiment comprises cyclosporin A, T-Det O-8®, and Eucalyptus oil.

Another embodiment comprises cyclosporin A, T-Det O-9®, and Eucalyptus oil.

Another embodiment comprises cyclosporin A, Tegin®, and Eucalyptus oil.

Another embodiment comprises cyclosporin A, Tergitol 15-S-12®, and Eucalyptus oil.

Another embodiment comprises cyclosporin A, Tergitol 15-S-15®, and Eucalyptus oil.

Another embodiment comprises cyclosporin A, Tergitol 15-S-20®, and Eucalyptus oil.

Another embodiment comprises cyclosporin A, Tergitol 15-S-20 (80AQ)®, and Eucalyptus oil.

Another embodiment comprises cyclosporin A, Tergitol 15-S-3®, and Eucalyptus oil.

Another embodiment comprises cyclosporin A, Tergitol 15-S-30®, and Eucalyptus oil.

Another embodiment comprises cyclosporin A, Tergitol 15-S-40®, and Eucalyptus oil.

Another embodiment comprises cyclosporin A, Tergitol 15-S-40 (70AQ)®, and Eucalyptus oil.

Another embodiment comprises cyclosporin A, Tergitol 15-S-5®, and Eucalyptus oil.

Another embodiment comprises cyclosporin A, Tergitol 15-S-7®, and Eucalyptus oil.

Another embodiment comprises cyclosporin A, Tergitol 15-S-9®, and Eucalyptus oil.

Another embodiment comprises cyclosporin A, Tergitol L-101®, and Eucalyptus oil.

Another embodiment comprises cyclosporin A, Tergitol L-61®, and Eucalyptus oil.

Another embodiment comprises cyclosporin A, Tergitol L-62®, and Eucalyptus oil.

Another embodiment comprises cyclosporin A, Tergitol L-64®, and Eucalyptus oil.

Another embodiment comprises cyclosporin A, Tergitol L-81®, and Eucalyptus oil.

Another embodiment comprises cyclosporin A, Tergitol NP-10®, and Eucalyptus oil.

Another embodiment comprises cyclosporin A, Tergitol NP-11®, and Eucalyptus oil.

Another embodiment comprises cyclosporin A, Tergitol NP-12®, and Eucalyptus oil.

Another embodiment comprises cyclosporin A, Tergitol NP-13®, and Eucalyptus oil.

Another embodiment comprises cyclosporin A, Tergitol NP-15®, and Eucalyptus oil.

Another embodiment comprises cyclosporin A, Tergitol NP-30®, and Eucalyptus oil.

Another embodiment comprises cyclosporin A, Tergitol NP-30 (70 AQ)®, and Eucalyptus oil.

Another embodiment comprises cyclosporin A, Tergitol NP-4®, and Eucalyptus oil.

Another embodiment comprises cyclosporin A, Tergitol NP-40®, and Eucalyptus oil.

Another embodiment comprises cyclosporin A, Tergitol NP-40 (70 AQ)®, and Eucalyptus oil.

Another embodiment comprises cyclosporin A, Tergitol NP-50 (70 AQ)®, and Eucalyptus oil.

Another embodiment comprises cyclosporin A, Tergitol NP-55 (70 AQ)®, and Eucalyptus oil.

Another embodiment comprises cyclosporin A, Tergitol NP-6®, and Eucalyptus oil.

Another embodiment comprises cyclosporin A, Tergitol NP-7®, and Eucalyptus oil.

Another embodiment comprises cyclosporin A, Tergitol NP-7 (70 AQ)®, and Eucalyptus oil.

Another embodiment comprises cyclosporin A, Tergitol NP-8®, and Eucalyptus oil.

Another embodiment comprises cyclosporin A, Tergitol NP-9®, and Eucalyptus oil.

Another embodiment comprises cyclosporin A, Tergitol NP-9.5®, and Eucalyptus oil.

Another embodiment comprises cyclosporin A, Tergitol TMN-10®, and Eucalyptus oil.

Another embodiment comprises cyclosporin A, Tergitol TMN-3®, and Eucalyptus oil.

Another embodiment comprises cyclosporin A, Tergitol TMN-6®, and Eucalyptus oil.

Another embodiment comprises cyclosporin A, Tergitol XD®, and Eucalyptus oil.

Another embodiment comprises cyclosporin A, Tergitol XH®, and Eucalyptus oil.

Another embodiment comprises cyclosporin A, Tergitol XJ®, and Eucalyptus oil.

Another embodiment comprises cyclosporin A, Tetronic 1107®, and Eucalyptus oil.

Another embodiment comprises cyclosporin A, Tetronic 1307®, and Eucalyptus oil.

Another embodiment comprises cyclosporin A, Tetronic 150R1®, and Eucalyptus oil.

Another embodiment comprises cyclosporin A, Tetronic 304®, and Eucalyptus oil.

Another embodiment comprises cyclosporin A, Tetronic 701®, and Eucalyptus oil.

Another embodiment comprises cyclosporin A, Tetronic 901g, and Eucalyptus oil.

Another embodiment comprises cyclosporin A, Tetronic 904®, and Eucalyptus oil.

Another embodiment comprises cyclosporin A, Tetronic 908®, and Eucalyptus oil.

Another embodiment comprises cyclosporin A, Tetronic 90R4®, and Eucalyptus oil.

Another embodiment comprises cyclosporin A, Tomadol 1-3®, and Eucalyptus oil.

Another embodiment comprises cyclosporin A, Tomadol 1-5®, and Eucalyptus oil.

Another embodiment comprises cyclosporin A, Tomadol 1-7®, and Eucalyptus oil.

Another embodiment comprises cyclosporin A, Tomadol 23-1®, and Eucalyptus oil.

Another embodiment comprises cyclosporin A, Tomadol 23-3®, and Eucalyptus oil.

Another embodiment comprises cyclosporin A, Tomadol 23-5®, and Eucalyptus oil.

Another embodiment comprises cyclosporin A, Tomadol 23-6.5®, and Eucalyptus oil.

Another embodiment comprises cyclosporin A, Tomadol 25-12®, and Eucalyptus oil.

Another embodiment comprises cyclosporin A, Tomadol 25-3®, and Eucalyptus oil.

Another embodiment comprises cyclosporin A, Tomadol 25-7®, and Eucalyptus oil.

Another embodiment comprises cyclosporin A, Tomadol 25-9®, and Eucalyptus oil.

Another embodiment comprises cyclosporin A, Tomadol 45-13®, and Eucalyptus oil.

Another embodiment comprises cyclosporin A, Tomadol 45-2.5®, and Eucalyptus oil.

Another embodiment comprises cyclosporin A, Tomadol 45-7®, and Eucalyptus oil.

Another embodiment comprises cyclosporin A, Tomadol 91-2.5®, and Eucalyptus oil.

Another embodiment comprises cyclosporin A, Tomadol 91-6®, and Eucalyptus oil.

Another embodiment comprises cyclosporin A, Tomadol 91-8®, and Eucalyptus oil.

Another embodiment comprises cyclosporin A, Triton X-100®, and Eucalyptus oil.

Another embodiment comprises cyclosporin A, Triton X-102®, and Eucalyptus oil.

Another embodiment comprises cyclosporin A, Triton X-114®, and Eucalyptus oil.

Another embodiment comprises cyclosporin A, Triton X-15®, and Eucalyptus oil.

Another embodiment comprises cyclosporin A, Triton X-165 (70%)®, and Eucalyptus oil.

Another embodiment comprises cyclosporin A, Triton X-207®, and Eucalyptus oil.

Another embodiment comprises cyclosporin A, Triton X-305 (70%)®, and Eucalyptus oil.

Another embodiment comprises cyclosporin A, Triton X-35®, and Eucalyptus oil.

Another embodiment comprises cyclosporin A, Triton X-405 (70%)®, and Eucalyptus oil.

Another embodiment comprises cyclosporin A, Triton X-45®, and Eucalyptus oil.

Another embodiment comprises cyclosporin A, Triton X-705 (70%)®, and Eucalyptus oil.

Another embodiment comprises cyclosporin A, Volpo S-10®, and Eucalyptus oil.

Another embodiment comprises cyclosporin A, Volpo S-2®, and Eucalyptus oil.

Another embodiment comprises cyclosporin A, Volpo S-20®, and Eucalyptus oil.

Another embodiment comprises cyclosporin A, Volpo-10®, and Eucalyptus oil.

Another embodiment comprises cyclosporin A, Volpo-20®, and Eucalyptus oil.

Another embodiment comprises cyclosporin A, Volpo-3®, and Eucalyptus oil.

Another embodiment comprises cyclosporin A, Volpo-5®, and Eucalyptus oil.

Another embodiment comprises cyclosporin A, Abex 2515®, and Sesame oil.

Another embodiment comprises cyclosporin A, Abex 2525/40®, and Sesame oil.

Another embodiment comprises cyclosporin A, Abex 2535®, and Sesame oil.

Another embodiment comprises cyclosporin A, Abex 2545®, and Sesame oil.

Another embodiment comprises cyclosporin A, Acconon E®, and Sesame oil.

Another embodiment comprises cyclosporin A, Alfonic 1216CO-9®, and Sesame oil.

Another embodiment comprises cyclosporin A, Alfonic 1412-3®, and Sesame oil.

Another embodiment comprises cyclosporin A, Alfonic TDA-12®, and Sesame oil.

Another embodiment comprises cyclosporin A, Alfonic TDA-3®, and Sesame oil.

Another embodiment comprises cyclosporin A, Alfonic TDA-4®, and Sesame oil.

Another embodiment comprises cyclosporin A, Alfonic TDA-6®, and Sesame oil.

Another embodiment comprises cyclosporin A, Alfonic TDA-7®, and Sesame oil.

Another embodiment comprises cyclosporin A, Alfonic TDA-8®, and Sesame oil.

Another embodiment comprises cyclosporin A, Alfonic TDA-9®, and Sesame oil.

Another embodiment comprises cyclosporin A, Alkamuls 400-DO®, and Sesame oil.

Another embodiment comprises cyclosporin A, Alkamuls 400-MO/E®, and Sesame oil.

Another embodiment comprises cyclosporin A, Ameroxol OE-10®, and Sesame oil.

Another embodiment comprises cyclosporin A, Ameroxol OE-2®, and Sesame oil.

Another embodiment comprises cyclosporin A, Ameroxol OE-20®, and Sesame oil.

Another embodiment comprises cyclosporin A, Armix 176®, and Sesame oil.

Another embodiment comprises cyclosporin A, Armix 180-C®, and Sesame oil.

Another embodiment comprises cyclosporin A, Armix 183®, and Sesame oil.

Another embodiment comprises cyclosporin A, Brij 35®, and Sesame oil.

Another embodiment comprises cyclosporin A, Brij 35 Liquid®, and Sesame oil.

Another embodiment comprises cyclosporin A, Brij 35 SP®, and Sesame oil.

Another embodiment comprises cyclosporin A, Canasol MJ 2109®, and Sesame oil.

Another embodiment comprises cyclosporin A, Canasol MJ 52®, and Sesame oil.

Another embodiment comprises cyclosporin A, Canasol R 3600®, and Sesame oil.

Another embodiment comprises cyclosporin A, Canasol R 3603®, and Sesame oil.

Another embodiment comprises cyclosporin A, Canasol R 4000 H®, and Sesame oil.

Another embodiment comprises cyclosporin A, Cedepal CA-210®, and Sesame oil.

Another embodiment comprises cyclosporin A, Cedepal CA-520®, and Sesame oil.

Another embodiment comprises cyclosporin A, Cedepal CA-630®, and Sesame oil.

Another embodiment comprises cyclosporin A, Cedepal CA-720®, and Sesame oil.

Another embodiment comprises cyclosporin A, Cedepal CA-890®, and Sesame oil.

Another embodiment comprises cyclosporin A, Cedepal CA-897®, and Sesame oil.

Another embodiment comprises cyclosporin A, Cedepal CO-210®, and Sesame oil.

Another embodiment comprises cyclosporin A, Cedepal CO-430®, and Sesame oil.

Another embodiment comprises cyclosporin A, Cedepal CO-530®, and Sesame oil.

Another embodiment comprises cyclosporin A, Cedepal CO-630®, and Sesame oil.

Another embodiment comprises cyclosporin A, Cedepal CO-710®, and Sesame oil.

Another embodiment comprises cyclosporin A, Cedepal CO-730®, and Sesame oil.

Another embodiment comprises cyclosporin A, Cedepal CO-880®, and Sesame oil.

Another embodiment comprises cyclosporin A, Cedepal CO-887®, and Sesame oil.

Another embodiment comprises cyclosporin A, Cedepal CO-890®, and Sesame oil.

Another embodiment comprises cyclosporin A, Cedepal CO-897®, and Sesame oil.

Another embodiment comprises cyclosporin A, Cedepal CO-970®, and Sesame oil.

Another embodiment comprises cyclosporin A, Cedepal CO-977®, and Sesame oil.

Another embodiment comprises cyclosporin A, Cedepal CO-990®, and Sesame oil.

Another embodiment comprises cyclosporin A, Cedepal CO-997®, and Sesame oil.

Another embodiment comprises cyclosporin A, Chemal BP 261®, and Sesame oil.

Another embodiment comprises cyclosporin A, Chemal BP 262®, and Sesame oil.

Another embodiment comprises cyclosporin A, Chemal BP 3172®, and Sesame oil.

Another embodiment comprises cyclosporin A, Chemal BP 3174®, and Sesame oil.

Another embodiment comprises cyclosporin A, Chemal DA-4®, and Sesame oil.

Another embodiment comprises cyclosporin A, Chemal DA-6®, and Sesame oil.

Another embodiment comprises cyclosporin A, Chemal DA-9®, and Sesame oil.

Another embodiment comprises cyclosporin A, Chemal LA-23®, and Sesame oil.

Another embodiment comprises cyclosporin A, Chemal LA-4®, and Sesame oil.

Another embodiment comprises cyclosporin A, Chemal LA-9®, and Sesame oil.

Another embodiment comprises cyclosporin A, Chemal OA-20G®, and Sesame oil.

Another embodiment comprises cyclosporin A, Chemal OA-5®, and Sesame oil.

Another embodiment comprises cyclosporin A, Chemal OA-9®, and Sesame oil.

Another embodiment comprises cyclosporin A, Chemal TDA-12®, and Sesame oil.

Another embodiment comprises cyclosporin A, Chemal TDA-15®, and Sesame oil.

Another embodiment comprises cyclosporin A, Chemal TDA-3®, and Sesame oil.

Another embodiment comprises cyclosporin A, Chemal TDA-6®, and Sesame oil.

Another embodiment comprises cyclosporin A, Chemal TDA-9®, and Sesame oil.

Another embodiment comprises cyclosporin A, Chemax CO-16®, and Sesame oil.

Another embodiment comprises cyclosporin A, Chemax CO-200/50®, and Sesame oil.

Another embodiment comprises cyclosporin A, Chemax CO-25®, and Sesame oil.

Another embodiment comprises cyclosporin A, Chemax CO-30®, and Sesame oil.

Another embodiment comprises cyclosporin A, Chemax CO-36®, and Sesame oil.

Another embodiment comprises cyclosporin A, Chemax CO-40®, and Sesame oil.

Another embodiment comprises cyclosporin A, Chemax CO-5®, and Sesame oil.

Another embodiment comprises cyclosporin A, Chemax CO-80®, and Sesame oil.

Another embodiment comprises cyclosporin A, Chemax DNP-150/50®, and Sesame oil.

Another embodiment comprises cyclosporin A, Chemax DNP-18®, and Sesame oil.

Another embodiment comprises cyclosporin A, Chemax DNP-8®, and Sesame oil.

Another embodiment comprises cyclosporin A, Chemax E 200 ML®, and Sesame oil.

Another embodiment comprises cyclosporin A, Chemax E 200 MO®, and Sesame oil.

Another embodiment comprises cyclosporin A, Chemax E 400 ML®, and Sesame oil.

Another embodiment comprises cyclosporin A, Chemax E 400 MO®, and Sesame oil.

Another embodiment comprises cyclosporin A, Chemax E 600 ML®, and Sesame oil.

Another embodiment comprises cyclosporin A, Chemax E 600 MO®, and Sesame oil.

Another embodiment comprises cyclosporin A, Chemax E 1000 MO®, and Sesame oil.

Another embodiment comprises cyclosporin A, Chemax E 1000 MS®, and Sesame oil.

Another embodiment comprises cyclosporin A, Chemax E 200 MS®, and Sesame oil.

Another embodiment comprises cyclosporin A, Chemax E 400 MS®, and Sesame oil.

Another embodiment comprises cyclosporin A, Chemax E600 MS®, and Sesame oil.

Another embodiment comprises cyclosporin A, Chemax HCO-16®, and Sesame oil.

Another embodiment comprises cyclosporin A, Chemax HCO-200/50®, and Sesame oil.

Another embodiment comprises cyclosporin A, Chemax HCO-25®, and Sesame oil.

Another embodiment comprises cyclosporin A, Chemax HCO-5®, and Sesame oil.

Another embodiment comprises cyclosporin A, Chemax TO-16®, and Sesame oil.

Another embodiment comprises cyclosporin A, Chemax TO-8®, and Sesame oil.

Another embodiment comprises cyclosporin A, Colalube 3409®, and Sesame oil.

Another embodiment comprises cyclosporin A, Colalube 3411®, and Sesame oil.

Another embodiment comprises cyclosporin A, Colalube 3413®, and Sesame oil.

Another embodiment comprises cyclosporin A, Colalube 3414®, and Sesame oil.

Another embodiment comprises cyclosporin A, Colalube 3415®, and Sesame oil.

Another embodiment comprises cyclosporin A, Colalube 3416®, and Sesame oil.

Another embodiment comprises cyclosporin A, Colalube 3417®, and Sesame oil.

Another embodiment comprises cyclosporin A, Delonic NPE-100®, and Sesame oil.

Another embodiment comprises cyclosporin A, Delonic NPE-30®, and Sesame oil.

Another embodiment comprises cyclosporin A, Delonic NPE-40®, and Sesame oil.

Another embodiment comprises cyclosporin A, Delonic OPE-10®, and Sesame oil.

Another embodiment comprises cyclosporin A, Delonic OPE-12®, and Sesame oil.

Another embodiment comprises cyclosporin A, Delonic OPE-30®, and Sesame oil.

Another embodiment comprises cyclosporin A, Delonic OPE-40®, and Sesame oil.

Another embodiment comprises cyclosporin A, Delonic OPE-5®, and Sesame oil.

Another embodiment comprises cyclosporin A, Delonic OPE-7.5®, and Sesame oil.

Another embodiment comprises cyclosporin A, DePeg 16-CO®, and Sesame oil.

Another embodiment comprises cyclosporin A, DePeg 25-C®, and Sesame oil.

Another embodiment comprises cyclosporin A, DePeg 30-CO®, and Sesame oil.

Another embodiment comprises cyclosporin A, DePeg 40-CO®, and Sesame oil.

Another embodiment comprises cyclosporin A, DePeg 5-CO®, and Sesame oil.

Another embodiment comprises cyclosporin A, DePeg 80-CO®, and Sesame oil.

Another embodiment comprises cyclosporin A, DeSonic S-100®, and Sesame oil.

Another embodiment comprises cyclosporin A, DeSonic S-114®, and Sesame oil.

Another embodiment comprises cyclosporin A, DeSonic S-405®, and Sesame oil.

Another embodiment comprises cyclosporin A, DeSonic S-45®, and Sesame oil.

Another embodiment comprises cyclosporin A, DeSonic TDA-9®, and Sesame oil.

Another embodiment comprises cyclosporin A, DeThox Acid O-14®, and Sesame oil.

Another embodiment comprises cyclosporin A, DeThox Acid O-16®, and Sesame oil.

Another embodiment comprises cyclosporin A, DeThox Acid O-9®, and Sesame oil.

Another embodiment comprises cyclosporin A, DeThox Acid TO-14®, and Sesame oil.

Another embodiment comprises cyclosporin A, DeThox Acid TO-16.5®, and Sesame oil.

Another embodiment comprises cyclosporin A, DeThox Acid TO-8.5®, and Sesame oil.

Another embodiment comprises cyclosporin A, Dur-Em 114®, and Sesame oil.

Another embodiment comprises cyclosporin A, Dur-Em 117®, and Sesame oil.

Another embodiment comprises cyclosporin A, Dur-Em 204®, and Sesame oil.

Another embodiment comprises cyclosporin A, Dur-Em 207®, and Sesame oil.

Another embodiment comprises cyclosporin A, Dur-Em 207E®, and Sesame oil.

Another embodiment comprises cyclosporin A, Emerest 2704®, and Sesame oil.

Another embodiment comprises cyclosporin A, Emerest 2712®, and Sesame oil.

Another embodiment comprises cyclosporin A, Emerest 2715®, and Sesame oil.

Another embodiment comprises cyclosporin A, Ethal EH-2®, and Sesame oil.

Another embodiment comprises cyclosporin A, Ethal EH-5®, and Sesame oil.

Another embodiment comprises cyclosporin A, Ethal OA-23®, and Sesame oil.

Another embodiment comprises cyclosporin A, Ethosperse CA-20®, and Sesame oil.

Another embodiment comprises cyclosporin A, Ethosperse LA-12®, and Sesame oil.

Another embodiment comprises cyclosporin A, Ethosperse LA-23®, and Sesame oil.

Another embodiment comprises cyclosporin A, Ethosperse LA-4®, and Sesame oil.

Another embodiment comprises cyclosporin A, Ethox 3095®, and Sesame oil.

Another embodiment comprises cyclosporin A, Ethox CO-16®, and Sesame oil.

Another embodiment comprises cyclosporin A, Ethox CO-200®, and Sesame oil.

Another embodiment comprises cyclosporin A, Ethox CO-25®, and Sesame oil.

Another embodiment comprises cyclosporin A, Ethox CO-30®, and Sesame oil.

Another embodiment comprises cyclosporin A, Ethox CO-36®, and Sesame oil.

Another embodiment comprises cyclosporin A, Ethox CO-40®, and Sesame oil.

Another embodiment comprises cyclosporin A, Ethox CO-5®, and Sesame oil.

Another embodiment comprises cyclosporin A, Ethox DO-14®, and Sesame oil.

Another embodiment comprises cyclosporin A, Ethox DO-9®, and Sesame oil.

Another embodiment comprises cyclosporin A, Ethox MA-8®, and Sesame oil.

Another embodiment comprises cyclosporin A, Ethox MI-14®, and Sesame oil.

Another embodiment comprises cyclosporin A, Ethox ML-14®, and Sesame oil.

Another embodiment comprises cyclosporin A, Ethox ML-5®, and Sesame oil.

Another embodiment comprises cyclosporin A, Ethox MO-14®, and Sesame oil.

Another embodiment comprises cyclosporin A, Ethox MO-9®, and Sesame oil.

Another embodiment comprises cyclosporin A, Ethox MS-14®, and Sesame oil.

Another embodiment comprises cyclosporin A, Ethox MS-23®, and Sesame oil.

Another embodiment comprises cyclosporin A, Ethox MS-40®, and Sesame oil.

Another embodiment comprises cyclosporin A, Ethox MS-8®, and Sesame oil.

Another embodiment comprises cyclosporin A, Ethox TO-16®, and Sesame oil.

Another embodiment comprises cyclosporin A, Ethox TO-8®, and Sesame oil.

Another embodiment comprises cyclosporin A, Ethylan SN-120®, and Sesame oil.

Another embodiment comprises cyclosporin A, Ethylan SN-70®, and Sesame oil.

Another embodiment comprises cyclosporin A, Ethylan SN-90®, and Sesame oil.

Another embodiment comprises cyclosporin A, Flexricin 13®, and Sesame oil.

Another embodiment comprises cyclosporin A, Flexricin 15®, and Sesame oil.

Another embodiment comprises cyclosporin A, Genapol 1261®, and Sesame oil.

Another embodiment comprises cyclosporin A, Generol 122 E25®, and Sesame oil.

Another embodiment comprises cyclosporin A, Glicepol 160®, and Sesame oil.

Another embodiment comprises cyclosporin A, Glicepol 560®, and Sesame oil.

Another embodiment comprises cyclosporin A, Glicepol GMS 20®, and Sesame oil.

Another embodiment comprises cyclosporin A, Hetoxide BN-13®, and Sesame oil.

Another embodiment comprises cyclosporin A, Hetoxide DNP-4®, and Sesame oil.

Another embodiment comprises cyclosporin A, Hetoxide DNP-9.6®, and Sesame oil.

Another embodiment comprises cyclosporin A, Hetoxide G-26®, and Sesame oil.

Another embodiment comprises cyclosporin A, Hetoxide G-7®, and Sesame oil.

Another embodiment comprises cyclosporin A, Hetoxide GT-20®, and Sesame oil.

Another embodiment comprises cyclosporin A, Hetoxide HC-40®, and Sesame oil.

Another embodiment comprises cyclosporin A, Hetoxol C-24®, and Sesame oil.

Another embodiment comprises cyclosporin A, Hetoxol CA-10®, and Sesame oil.

Another embodiment comprises cyclosporin A, Hetoxol CA-2®, and Sesame oil.

Another embodiment comprises cyclosporin A, Hetoxol CA-20®, and Sesame oil.

Another embodiment comprises cyclosporin A, Hetoxol CAWS®, and Sesame oil.

Another embodiment comprises cyclosporin A, Hetoxol CD-3®, and Sesame oil.

Another embodiment comprises cyclosporin A, Hetoxol CD-4®, and Sesame oil.

Another embodiment comprises cyclosporin A, Hetoxol CS-15®, and Sesame oil.

Another embodiment comprises cyclosporin A, Hetoxol CS-20®, and Sesame oil.

Another embodiment comprises cyclosporin A, Hetoxol CS-20D®, and Sesame oil.

Another embodiment comprises cyclosporin A, Hetoxol CS-25®, and Sesame oil.

Another embodiment comprises cyclosporin A, Hetoxol CS-50®, and Sesame oil.

Another embodiment comprises cyclosporin A, Hetoxol CS-9®, and Sesame oil.

Another embodiment comprises cyclosporin A, Hetoxol CSA-15®, and Sesame oil.

Another embodiment comprises cyclosporin A, Hetoxol D®, and Sesame oil.

Another embodiment comprises cyclosporin A, Hetoxol G®, and Sesame oil.

Another embodiment comprises cyclosporin A, Hetoxol J®, and Sesame oil.

Another embodiment comprises cyclosporin A, Hetoxol L®, and Sesame oil.

Another embodiment comprises cyclosporin A, Hetoxol L-23®, and Sesame oil.

Another embodiment comprises cyclosporin A, Hetoxol L-4®, and Sesame oil.

Another embodiment comprises cyclosporin A, Hetoxol L-9®, and Sesame oil.

Another embodiment comprises cyclosporin A, Hetoxol LS-9®, and Sesame oil.

Another embodiment comprises cyclosporin A, Hetoxol M-3®, and Sesame oil.

Another embodiment comprises cyclosporin A, Hetoxol OA-10 Special®, and Sesame oil.

Another embodiment comprises cyclosporin A, Hetoxol OA-20 Special®, and Sesame oil.

Another embodiment comprises cyclosporin A, Hetoxol OA-3 Special®, and Sesame oil.

Another embodiment comprises cyclosporin A, Hetoxol OA-5 Special®, and Sesame oil.

Another embodiment comprises cyclosporin A, Hetoxol OL-10®, and Sesame oil.

Another embodiment comprises cyclosporin A, Hetoxol OL-2®, and Sesame oil.

Another embodiment comprises cyclosporin A, Hetoxol OL-23®, and Sesame oil.

Another embodiment comprises cyclosporin A, Hetoxol OL-4®, and Sesame oil.

Another embodiment comprises cyclosporin A, Hetoxol OL-40®, and Sesame oil.

Another embodiment comprises cyclosporin A, Hetoxol OL-5®, and Sesame oil.

Another embodiment comprises cyclosporin A, Hetoxol PLA®, and Sesame oil.

Another embodiment comprises cyclosporin A, Hetoxol SP-15®, and Sesame oil.

Another embodiment comprises cyclosporin A, Hetoxol STA-10®, and Sesame oil.

Another embodiment comprises cyclosporin A, Hetoxol STA-2®, and Sesame oil.

Another embodiment comprises cyclosporin A, Hetoxol STA-30®, and Sesame oil.

Another embodiment comprises cyclosporin A, Hetoxol TD-12®, and Sesame oil.

Another embodiment comprises cyclosporin A, Hetoxol TD-18®, and Sesame oil.

Another embodiment comprises cyclosporin A, Hetoxol TD-3®, and Sesame oil.

Another embodiment comprises cyclosporin A, Hetoxol TD-6®, and Sesame oil.

Another embodiment comprises cyclosporin A, Hetoxol TD-9®, and Sesame oil.

Another embodiment comprises cyclosporin A, Hetoxol TDEP-15®, and Sesame oil.

Another embodiment comprises cyclosporin A, Hetoxol TDEP-63®, and Sesame oil.

Another embodiment comprises cyclosporin A, Iconol 24-12®, and Sesame oil.

Another embodiment comprises cyclosporin A, Iconol 24-23®, and Sesame oil.

Another embodiment comprises cyclosporin A, Iconol 24-4®, and Sesame oil.

Another embodiment comprises cyclosporin A, Iconol DA-4®, and Sesame oil.

Another embodiment comprises cyclosporin A, Iconol DA-6®, and Sesame oil.

Another embodiment comprises cyclosporin A, Iconol TDA-10®, and Sesame oil.

Another embodiment comprises cyclosporin A, Iconol TDA-3®, and Sesame oil.

Another embodiment comprises cyclosporin A, Iconol TDA-6®, and Sesame oil.

Another embodiment comprises cyclosporin A, Iconol TDA-8-90%®, and Sesame oil.

Another embodiment comprises cyclosporin A, Iconol TDA-9®, and Sesame oil.

Another embodiment comprises cyclosporin A, Igepal CA-210®and Sesame oil.

Another embodiment comprises cyclosporin A, Igepal CA-407®, and Sesame oil.

Another embodiment comprises cyclosporin A, Igepal CA-420®, and Sesame oil.

Another embodiment comprises cyclosporin A, Igepal CA-520®, and Sesame oil.

Another embodiment comprises cyclosporin A, Igepal CA-620®, and Sesame oil.

Another embodiment comprises cyclosporin A, Igepal CA-630®, and Sesame oil.

Another embodiment comprises cyclosporin A, Igepal CA-720®, and Sesame oil.

Another embodiment comprises cyclosporin A, Igepal CA-877®, and Sesame oil.

Another embodiment comprises cyclosporin A, Igepal CA-887®, and Sesame oil.

Another embodiment comprises cyclosporin A, Igepal CA-897®, and Sesame oil.

Another embodiment comprises cyclosporin A, Igepal CO-210®, and Sesame oil.

Another embodiment comprises cyclosporin A, Igepal CO-430®, and Sesame oil.

Another embodiment comprises cyclosporin A, Igepal CO-520®, and Sesame oil.

Another embodiment comprises cyclosporin A, Igepal CO-530®, and Sesame oil.

Another embodiment comprises cyclosporin A, Igepal CO-610®, and Sesame oil.

Another embodiment comprises cyclosporin A, Igepal CO-630®, and Sesame oil.

Another embodiment comprises cyclosporin A, Igepal CO-630 Special®, and Sesame oil.

Another embodiment comprises cyclosporin A, Igepal CO-660®, and Sesame oil.

Another embodiment comprises cyclosporin A, Igepal CO-710®, and Sesame oil.

Another embodiment comprises cyclosporin A, Igepal CO-720®, and Sesame oil.

Another embodiment comprises cyclosporin A, Igepal CO-730®, and Sesame oil.

Another embodiment comprises cyclosporin A, Igepal CO-738®, and Sesame oil.

Another embodiment comprises cyclosporin A, Igepal CO-850®, and Sesame oil.

Another embodiment comprises cyclosporin A, Igepal CO-880®, and Sesame oil.

Another embodiment comprises cyclosporin A, Igepal CO-887®, and Sesame oil.

Another embodiment comprises cyclosporin A, Igepal CO-890®, and Sesame oil.

Another embodiment comprises cyclosporin A, Igepal CO-897®, and Sesame oil.

Another embodiment comprises cyclosporin A, Igepal CO-970®, and Sesame oil.

Another embodiment comprises cyclosporin A, Igepal CO-977®, and Sesame oil.

Another embodiment comprises cyclosporin A, Igepal CO-987®, and Sesame oil.

Another embodiment comprises cyclosporin A, Igepal CO-990 FLK®, and Sesame oil.

Another embodiment comprises cyclosporin A, Igepal CO-997®, and Sesame oil.

Another embodiment comprises cyclosporin A, Igepal DM-430®, and Sesame oil.

Another embodiment comprises cyclosporin A, Igepal DM-530®, and Sesame oil.

Another embodiment comprises cyclosporin A, Igepal DM-710®, and Sesame oil.

Another embodiment comprises cyclosporin A, Igepal DM-730®, and Sesame oil.

Another embodiment comprises cyclosporin A, Igepal DM-880®, and Sesame oil.

Another embodiment comprises cyclosporin A, Igepal DM-970 FLK®, and Sesame oil.

Another embodiment comprises cyclosporin A, Igepal OD-410®, and Sesame oil.

Another embodiment comprises cyclosporin A, Igepal RC-520®, and Sesame oil.

Another embodiment comprises cyclosporin A, Igepal RC-620®, and Sesame oil.

Another embodiment comprises cyclosporin A, Igepal RC-630®, and Sesame oil.

Another embodiment comprises cyclosporin A, Lexemul CS-20®, and Sesame oil.

Another embodiment comprises cyclosporin A, Lipo GMS 450®, and Sesame oil.

Another embodiment comprises cyclosporin A, Lipo Polyglycol 1000®, and Sesame oil.

Another embodiment comprises cyclosporin A, Lipo Polyglycol 200®, and Sesame oil.

Another embodiment comprises cyclosporin A, Lipo Polyglycol 300®, and Sesame oil.

Another embodiment comprises cyclosporin A, Lipo Polyglycol 3350®, and Sesame oil.

Another embodiment comprises cyclosporin A, Lipo Polyglycol 400®, and Sesame oil.

Another embodiment comprises cyclosporin A, Lipo Polyglycol 600®, and Sesame oil.

Another embodiment comprises cyclosporin A, Lipocol C-10®, and Sesame oil.

Another embodiment comprises cyclosporin A, Lipocol C-2®, and Sesame oil.

Another embodiment comprises cyclosporin A, Lipocol C-20®, and Sesame oil.

Another embodiment comprises cyclosporin A, Lipocol L-12®, and Sesame oil.

Another embodiment comprises cyclosporin A, Lipocol L-23®, and Sesame oil.

Another embodiment comprises cyclosporin A, Lipocol L-4®, and Sesame oil.

Another embodiment comprises cyclosporin A, Lipocol O-10®, and Sesame oil.

Another embodiment comprises cyclosporin A, Lipocol O-2®, and Sesame oil.

Another embodiment comprises cyclosporin A, Lipocol O-20®, and Sesame oil.

Another embodiment comprises cyclosporin A, Lipocol O-5®, and Sesame oil.

Another embodiment comprises cyclosporin A, Lipocol S-10®, and Sesame oil.

Another embodiment comprises cyclosporin A, Lipocol S-2®, and Sesame oil.

Another embodiment comprises cyclosporin A, Lipocol S-20®, and Sesame oil.

Another embodiment comprises cyclosporin A, Lipocol SC-15®, and Sesame oil.

Another embodiment comprises cyclosporin A, Lipocol SC-20®, and Sesame oil.

Another embodiment comprises cyclosporin A, Lipocol SC-4®, and Sesame oil.

Another embodiment comprises cyclosporin A, Lipocol TD-12®, and Sesame oil.

Another embodiment comprises cyclosporin A, Lumulse 40-O K®, and Sesame oil.

Another embodiment comprises cyclosporin A, Lumulse 42-O K®, and Sesame oil.

Another embodiment comprises cyclosporin A, Lumulse GMT-40®, and Sesame oil.

Another embodiment comprises cyclosporin A, Lumulse PEG 1450®, and Sesame oil.

Another embodiment comprises cyclosporin A, Lumulse PEG 1450 NF®, and Sesame oil.

Another embodiment comprises cyclosporin A, Lumulse PEG 8000®, and Sesame oil.

Another embodiment comprises cyclosporin A, Lumulse PGO®, and Sesame oil.

Another embodiment comprises cyclosporin A, Lumulse POE (2) Oleyl Amine®, and Sesame oil.

Another embodiment comprises cyclosporin A, Magrabar PGO-104®, and Sesame oil.

Another embodiment comprises cyclosporin A, Mapeg 200 ML®, and Sesame oil.

Another embodiment comprises cyclosporin A, Mapeg 400 MO®, and Sesame oil.

Another embodiment comprises cyclosporin A, Mapeg 400 MOT®, and Sesame oil.

Another embodiment comprises cyclosporin A, Mapeg 400 DO®, and Sesame oil.

Another embodiment comprises cyclosporin A, Mapeg 400 DOT®, and Sesame oil.

Another embodiment comprises cyclosporin A, Mapeg 600DOT®, and. Sesame oil.

Another embodiment comprises cyclosporin A, Marlox B 24/80®, and Sesame oil.

Another embodiment comprises cyclosporin A, Marlox FK 86®, and Sesame oil.

Another embodiment comprises cyclosporin A, Marlox MO 154®, and Sesame oil.

Another embodiment comprises cyclosporin A, Mazox CDA CG®, and Sesame oil.

Another embodiment comprises cyclosporin A, Monoglycerides Glyceryl Monestearate®, and Sesame oil.

Another embodiment comprises cyclosporin A, Myrj 45®, and Sesame oil.

Another embodiment comprises cyclosporin A, Myrj 52®, and Sesame oil.

Another embodiment comprises cyclosporin A, Myrj 52S®, and Sesame oil.

Another embodiment comprises cyclosporin A, Myrj 53®, and Sesame oil.

Another embodiment comprises cyclosporin A, Myrj 59®, and Sesame oil.

Another embodiment comprises cyclosporin A, Norfox NP-12®, and Sesame oil.

Another embodiment comprises cyclosporin A, Norfox NP-4®, and Sesame oil.

Another embodiment comprises cyclosporin A, Norfox NP-6®, and Sesame oil.

Another embodiment comprises cyclosporin A, Norfox NP-887®, and Sesame oil.

Another embodiment comprises cyclosporin A, Norfox NP-9®, and Sesame oil.

Another embodiment comprises cyclosporin A, Norfox NP-977®, and Sesame oil.

Another embodiment comprises cyclosporin A, Norfox OP-100®, and Sesame oil.

Another embodiment comprises cyclosporin A, Norfox OP-102®, and Sesame oil.

Another embodiment comprises cyclosporin A, Norfox OP-114®, and Sesame oil.

Another embodiment comprises cyclosporin A, Norfox OP-45®, and Sesame oil.

Another embodiment comprises cyclosporin A, Pegosperse 100-L®, and Sesame oil.

Another embodiment comprises cyclosporin A, Pegosperse 100-O®, and Sesame oil.

Another embodiment comprises cyclosporin A, Pegosperse 100S®, and Sesame oil.

Another embodiment comprises cyclosporin A, Pegosperse 1500-MS®, and Sesame oil.

Another embodiment comprises cyclosporin A, Pegosperse 1750-MS®, and Sesame oil.

Another embodiment comprises cyclosporin A, Pegosperse 200 DL®, and Sesame oil.

Another embodiment comprises cyclosporin A, Pegosperse 200-ML®, and Sesame oil.

Another embodiment comprises cyclosporin A, Pegosperse 400-DL®, and Sesame oil.

Another embodiment comprises cyclosporin A, Pegosperse 400-DO®, and Sesame oil.

Another embodiment comprises cyclosporin A, Pegosperse 400-DS®, and Sesame oil.

Another embodiment comprises cyclosporin A, Pegosperse 400-ML®, and Sesame oil.

Another embodiment comprises cyclosporin A, Pegosperse 400-MO®, and Sesame oil.

Another embodiment comprises cyclosporin A, Pegosperse 400-MOT®, and Sesame oil.

Another embodiment comprises cyclosporin A, Pegosperse 400-MS®, and Sesame oil.

Another embodiment comprises cyclosporin A, Pegosperse 50-DS®, and Sesame oil.

Another embodiment comprises cyclosporin A, Pegosperse 50-MS®, and Sesame oil.

Another embodiment comprises cyclosporin A, Pegosperse 600-ML®, and Sesame oil.

Another embodiment comprises cyclosporin A, Pegosperse 600-MS®, and Sesame oil.

Another embodiment comprises cyclosporin A, Plurafac A-38®, and Sesame oil.

Another embodiment comprises cyclosporin A, Plurafac B-25-5®, and Sesame oil.

Another embodiment comprises cyclosporin A, Plurafac B-26®, and Sesame oil.

Another embodiment comprises cyclosporin A, Plurafac D-25®, and Sesame oil.

Another embodiment comprises cyclosporin A, Plurafac LF 131®, and Sesame oil.

Another embodiment comprises cyclosporin A, Plurafac LF 4030®, and Sesame oil.

Another embodiment comprises cyclosporin A, Plurafac LF 7000®, and Sesame oil.

Another embodiment comprises cyclosporin A, Plurafac RA-20®, and Sesame oil.

Another embodiment comprises cyclosporin A, Plurafac RA-30®, and Sesame oil.

Another embodiment comprises cyclosporin A, Plurafac RA-40®, and Sesame oil.

Another embodiment comprises cyclosporin A, Pluronic 10R5®, and Sesame oil.

Another embodiment comprises cyclosporin A, Pluronic 17R2®, and Sesame oil.

Another embodiment comprises cyclosporin A, Pluronic 17R4®, and Sesame oil.

Another embodiment comprises cyclosporin A, Pluronic 25R2®, and Sesame oil.

Another embodiment comprises cyclosporin A, Pluronic 25R4®, and Sesame oil.

Another embodiment comprises cyclosporin A, Pluronic 31R1®, and Sesame oil.

Another embodiment comprises cyclosporin A, Pluronic F108®, and Sesame oil.

Another embodiment comprises cyclosporin A, Pluronic F127®, and Sesame oil.

Another embodiment comprises cyclosporin A, Pluronic F38®, and Sesame oil.

Another embodiment comprises cyclosporin A, Pluronic F68®, and Sesame oil.

Another embodiment comprises cyclosporin A, Pluronic F68LF®, and Sesame oil.

Another embodiment comprises cyclosporin A, Pluronic F77®, and Sesame oil.

Another embodiment comprises cyclosporin A, Pluronic F87®, and Sesame oil.

Another embodiment comprises cyclosporin A, Pluronic F88®, and Sesame oil.

Another embodiment comprises cyclosporin A, Pluronic F98®, and Sesame oil.

Another embodiment comprises cyclosporin A, Pluronic L 10®, and Sesame oil.

Another embodiment comprises cyclosporin A, Pluronic L 101®, and Sesame oil.

Another embodiment comprises cyclosporin A, Pluronic L 121®, and Sesame oil.

Another embodiment comprises cyclosporin A, Pluronic L31®, and Sesame oil.

Another embodiment comprises cyclosporin A, Pluronic L35®, and Sesame oil.

Another embodiment comprises cyclosporin A, Pluronic L43®, and Sesame oil.

Another embodiment comprises cyclosporin A, Pluronic L44®, and Sesame oil.

Another embodiment comprises cyclosporin A, Pluronic L61®, and Sesame oil.

Another embodiment comprises cyclosporin A, Pluronic L62®, and Sesame oil.

Another embodiment comprises cyclosporin A, Pluronic L62D®, and Sesame oil.

Another embodiment comprises cyclosporin A, Pluronic L62LF®, and Sesame oil.

Another embodiment comprises cyclosporin A, Pluronic L64®, and Sesame oil.

Another embodiment comprises cyclosporin A, Pluronic L81®, and Sesame oil.

Another embodiment comprises cyclosporin A, Pluronic L92®, and Sesame oil.

Another embodiment comprises cyclosporin A, Pluronic P103®, and Sesame oil.

Another embodiment comprises cyclosporin A, Pluronic P104®, and Sesame oil.

Another embodiment comprises cyclosporin A, Pluronic P105®, and Sesame oil.

Another embodiment comprises cyclosporin A, Pluronic P123®, and Sesame oil.

Another embodiment comprises cyclosporin A, Pluronic P65®, and Sesame oil.

Another embodiment comprises cyclosporin A, Pluronic P84®, and Sesame oil.

Another embodiment comprises cyclosporin A, Pluronic P85®, and Sesame oil.

Another embodiment comprises cyclosporin A, Polyaldo 10-2-P®, and Sesame oil.

Another embodiment comprises cyclosporin A, Polyaldo DGDO®, and Sesame oil.

Another embodiment comprises cyclosporin A, Polyaldo HGDS®, and Sesame oil.

Another embodiment comprises cyclosporin A, Polyaldo TGMS®, and Sesame oil.

Another embodiment comprises cyclosporin A, Protegin V®, and Sesame oil.

Another embodiment comprises cyclosporin A, Protegin W®, and Sesame oil.

Another embodiment comprises cyclosporin A, Protegin WX®, and Sesame oil.

Another embodiment comprises cyclosporin A, Protegin XV®, and Sesame oil.

Another embodiment comprises cyclosporin A, Rhodasurf BC-420®, and Sesame oil.

Another embodiment comprises cyclosporin A, Rhodasurf BC-610®, and Sesame oil.

Another embodiment comprises cyclosporin A, Rhodasurf BC-630®, and Sesame oil.

Another embodiment comprises cyclosporin A, Rhodasurf BC-720®, and Sesame oil.

Another embodiment comprises cyclosporin A, Rhodasurf BC-84®, and Sesame oil.

Another embodiment comprises cyclosporin A, Rhodasurf L 1®, and Sesame oil.

Another embodiment comprises cyclosporin A, Rhodasurf L 12®, and Sesame oil.

Another embodiment comprises cyclosporin A, Rhodasurf L 2®, and Sesame oil.

Another embodiment comprises cyclosporin A, Rhodasurf L 20®, and Sesame oil.

Another embodiment comprises cyclosporin A, Rhodasurf L 3®, and Sesame oil.

Another embodiment comprises cyclosporin A, Rhodasurf L 30®, and Sesame oil.

Another embodiment comprises cyclosporin A, Rhodasurf L 4®, and Sesame oil.

Another embodiment comprises cyclosporin A, Rhodasurf L 50®, and Sesame oil.

Another embodiment comprises cyclosporin A, Rhodasurf L 7/90®, and Sesame oil.

Another embodiment comprises cyclosporin A, Rhodasurf LA-3®, and Sesame oil.

Another embodiment comprises cyclosporin A, Rhodasurf LA-7®, and Sesame oil.

Another embodiment comprises cyclosporin A, Rhodasurf LAN-23®, and Sesame oil.

Another embodiment comprises cyclosporin A, Rhodasurf ON-870®, and Sesame oil.

Another embodiment comprises cyclosporin A, Rhodasurf ON-877®, and Sesame oil.

Another embodiment comprises cyclosporin A, Rhodasurf TDA-8/5®, and Sesame oil.

Another embodiment comprises cyclosporin A, Ritapro 100®, and Sesame oil.

Another embodiment comprises cyclosporin A, Ritapro 165®, and Sesame oil.

Another embodiment comprises cyclosporin A, Ritapro 200®, and Sesame oil.

Another embodiment comprises cyclosporin A, Ritapro 300®, and Sesame oil.

Another embodiment comprises cyclosporin A, Ritoleth 10®, and Sesame oil.

Another embodiment comprises cyclosporin A, Ritoleth 2®, and Sesame oil.

Another embodiment comprises cyclosporin A, Ritoleth 20®, and Sesame oil.

Another embodiment comprises cyclosporin A, Ritoleth 5®, and Sesame oil.

Another embodiment comprises cyclosporin A, Santone 10-10-0®, and Sesame oil.

Another embodiment comprises cyclosporin A, Santone 3-1-S XTR®, and Sesame oil.

Another embodiment comprises cyclosporin A, Santone 3-1-SH®, and Sesame oil.

Another embodiment comprises cyclosporin A, Santone 8-1-O®, and Sesame oil.

Another embodiment comprises cyclosporin A, Solulan 16®, and Sesame oil.

Another embodiment comprises cyclosporin A, Solulan 75®, and Sesame oil.

Another embodiment comprises cyclosporin A, Solulan C-24®, and Sesame oil.

Another embodiment comprises cyclosporin A, Solulan L-575®, and Sesame oil.

Another embodiment comprises cyclosporin A, Standamox CAW®, and Sesame oil.

Another embodiment comprises cyclosporin A, Surfine AZI-A®, and Sesame oil.

Another embodiment comprises cyclosporin A, Surfonic CO-15®, and Sesame oil.

Another embodiment comprises cyclosporin A, Surfonic CO-25®, and Sesame oil.

Another embodiment comprises cyclosporin A, Surfonic CO-30®, and Sesame oil.

Another embodiment comprises cyclosporin A, Surfonic CO-36®, and Sesame oil.

Another embodiment comprises cyclosporin A, Surfonic CO-42®, and Sesame oil.

Another embodiment comprises cyclosporin A, Surfonic E400-M®, and Sesame oil.

Another embodiment comprises cyclosporin A, Surfonic L12-3®, and Sesame oil.

Another embodiment comprises cyclosporin A, Surfonic L12-6®, and Sesame oil.

Another embodiment comprises cyclosporin A, Surfonic L12-8®, and Sesame oil.

Another embodiment comprises cyclosporin A, Surfonic L24-12®, and Sesame oil.

Another embodiment comprises cyclosporin A, Surfonic L24-2®, and Sesame oil.

Another embodiment comprises cyclosporin A, Surfonic 24-22®, and Sesame oil.

Another embodiment comprises cyclosporin A, Surfonic L24-3®, and Sesame oil.

Another embodiment comprises cyclosporin A, Surfonic L24-4®, and Sesame oil.

Another embodiment comprises cyclosporin A, Surfonic L24-5®, and Sesame oil.

Another embodiment comprises cyclosporin A, Surfonic L24-7®, and Sesame oil.

Another embodiment comprises cyclosporin A, Surfonic L24-7.1®, and Sesame oil.

Another embodiment comprises cyclosporin A, Surfonic L24-9®, and Sesame oil.

Another embodiment comprises cyclosporin A, Surfonic LF-17®, and Sesame oil.

Another embodiment comprises cyclosporin A, Surfonic LF-18®, and Sesame oil.

Another embodiment comprises cyclosporin A, Surfonic LF-37®, and Sesame oil.

Another embodiment comprises cyclosporin A, Surfonic LF-41®, and Sesame oil.

Another embodiment comprises cyclosporin A, Surfonic LF-42®, and Sesame oil.

Another embodiment comprises cyclosporin A, Surfonic LF-47®, and Sesame oil.

Another embodiment comprises cyclosporin A, Surfonic LF-68®, and Sesame oil.

Another embodiment comprises cyclosporin A, Surfonic N-10®, and Sesame oil.

Another embodiment comprises cyclosporin A, Surfonic N-100®, and Sesame oil.

Another embodiment comprises cyclosporin A, Surfonic N-102®, and Sesame oil.

Another embodiment comprises cyclosporin A, Surfonic N-120®, and Sesame oil.

Another embodiment comprises cyclosporin A, Surfonic N-150®, and Sesame oil.

Another embodiment comprises cyclosporin A, Surfonic N-200®, and Sesame oil.

Another embodiment comprises cyclosporin A, Surfonic N-300®, and Sesame oil.

Another embodiment comprises cyclosporin A, Surfonic N-31.5®, and Sesame oil.

Another embodiment comprises cyclosporin A, Surfonic N-40®, and Sesame oil.

Another embodiment comprises cyclosporin A, Surfonic N-400®, and Sesame oil.

Another embodiment comprises cyclosporin A, Surfonic N-60®, and Sesame oil.

Another embodiment comprises cyclosporin A, Surfonic N-85®, and Sesame oil.

Another embodiment comprises cyclosporin A, Surfonic N-95®, and Sesame oil.

Another embodiment comprises cyclosporin A, Surfonic NB-18®, and Sesame oil.

Another embodiment comprises cyclosporin A, Surfonic NB-23®, and Sesame oil.

Another embodiment comprises cyclosporin A, Surfonic NB-25®, and Sesame oil.

Another embodiment comprises cyclosporin A, Surfonic NB-307®, and Sesame oil.

Another embodiment comprises cyclosporin A, Surfonic NB-31®, and Sesame oil.

Another embodiment comprises cyclosporin A, Surfonic NB-41®, and Sesame oil.

Another embodiment comprises cyclosporin A, Surfonic NB-43®, and Sesame oil.

Another embodiment comprises cyclosporin A, Surfonic OP-100®, and Sesame oil.

Another embodiment comprises cyclosporin A, Surfonic OP-120®, and Sesame oil.

Another embodiment comprises cyclosporin A, Surfonic OP-15®, and Sesame oil.

Another embodiment comprises cyclosporin A, Surfonic OP-35®, and Sesame oil.

Another embodiment comprises cyclosporin A, Surfonic OP-70®, and Sesame oil.

Another embodiment comprises cyclosporin A, Surfonic P1®, and Sesame oil.

Another embodiment comprises cyclosporin A, Surfonic P3®, and Sesame oil.

Another embodiment comprises cyclosporin A, Surfonic P5®, and Sesame oil.

Another embodiment comprises cyclosporin A, Surfonic P6®, and Sesame oil.

Another embodiment comprises cyclosporin A, Surfonic POA-17R2®, and Sesame oil.

Another embodiment comprises cyclosporin A, Surfonic POA-25R2®, and Sesame oil.

Another embodiment comprises cyclosporin A, Surfonic TDA-11®, and Sesame oil.

Another embodiment comprises cyclosporin A, Surfonic TDA-3B®, and Sesame oil.

Another embodiment comprises cyclosporin A, Surfonic TDA-6®, and Sesame oil.

Another embodiment comprises cyclosporin A, Surfonic TDA-6/88®, and Sesame oil.

Another embodiment comprises cyclosporin A, Surfonic TDA-8®, and Sesame oil.

Another embodiment comprises cyclosporin A, Surfonic TDA-8/90®, and Sesame oil.

Another embodiment comprises cyclosporin A, Surfonic TDA-9®, and Sesame oil.

Another embodiment comprises cyclosporin A, Surfonic TX-CCN®, and Sesame oil.

Another embodiment comprises cyclosporin A, Tagat CH 40®, and Sesame oil.

Another embodiment comprises cyclosporin A, Tagat CH 60®, and Sesame oil.

Another embodiment comprises cyclosporin A, Tagat L 2®, and Sesame oil.

Another embodiment comprises cyclosporin A, Tagat S®, and Sesame oil.

Another embodiment comprises cyclosporin A, Tagat S 2®, and Sesame oil.

Another embodiment comprises cyclosporin A, Tally 100 Plus®, and Sesame oil.

Another embodiment comprises cyclosporin A, T-Det A106®, and Sesame oil.

Another embodiment comprises cyclosporin A, T-Det A109®, and Sesame oil.

Another embodiment comprises cyclosporin A, T-Det A136®, and Sesame oil.

Another embodiment comprises cyclosporin A, T-Det A139®, and Sesame oil.

Another embodiment comprises cyclosporin A, T-Det A2412®, and Sesame oil.

Another embodiment comprises cyclosporin A, T-Det A243®, and Sesame oil.

Another embodiment comprises cyclosporin A, T-Det A247®, and Sesame oil.

Another embodiment comprises cyclosporin A, T-Det A249®, and Sesame oil.

Another embodiment comprises cyclosporin A, T-Det A467®, and Sesame oil.

Another embodiment comprises cyclosporin A, T-Det COE®, and Sesame oil.

Another embodiment comprises cyclosporin A, T-Det DD-10®, and Sesame oil.

Another embodiment comprises cyclosporin A, T-Det DD-5®, and Sesame oil.

Another embodiment comprises cyclosporin A, T-Det DD-7®, and Sesame oil.

Another embodiment comprises cyclosporin A, T-Det EPO-104®, and Sesame oil.

Another embodiment comprises cyclosporin A, T-Det EPO-61®, and Sesame oil.

Another embodiment comprises cyclosporin A, T-Det EPO-62®, and Sesame oil.

Another embodiment comprises cyclosporin A, T-Det EPO-64®, and Sesame oil.

Another embodiment comprises cyclosporin A, T-Det N-1.5®, and Sesame oil.

Another embodiment comprises cyclosporin A, T-Det N-10.5®, and Sesame oil.

Another embodiment comprises cyclosporin A, T-Det N-100®, and Sesame oil.

Another embodiment comprises cyclosporin A, T-Det N-1007®, and Sesame oil.

Another embodiment comprises cyclosporin A, T-Det N-12®, and Sesame oil.

Another embodiment comprises cyclosporin A, T-Det N-14®, and Sesame oil.

Another embodiment comprises cyclosporin A, T-Det N-20®, and Sesame oil.

Another embodiment comprises cyclosporin A, T-Det N-30®, and Sesame oil.

Another embodiment comprises cyclosporin A, T-Det N-307®, and Sesame oil.

Another embodiment comprises cyclosporin A, T-Det N-4®, and Sesame oil.

Another embodiment comprises cyclosporin A, T-Det N-40®, and Sesame oil.

Another embodiment comprises cyclosporin A, T-Det N-407®, and Sesame oil.

Another embodiment comprises cyclosporin A, T-Det N-50®, and Sesame oil.

Another embodiment comprises cyclosporin A, T-Det N-507®, and Sesame oil.

Another embodiment comprises cyclosporin A, T-Det N-6®, and Sesame oil.

Another embodiment comprises cyclosporin A, T-Det N-70®, and Sesame oil.

Another embodiment comprises cyclosporin A, T-Det N-8®, and Sesame oil.

Another embodiment comprises cyclosporin A, T-Det N-9.5®, and Sesame oil.

Another embodiment comprises cyclosporin A, T-Det O-12®, and Sesame oil.

Another embodiment comprises cyclosporin A, T-Det O-165®, and Sesame oil.

Another embodiment comprises cyclosporin A, T-Det O-307®, and Sesame oil.

Another embodiment comprises cyclosporin A, T-Det O-40®, and Sesame oil.

Another embodiment comprises cyclosporin A, T-Det O-407®, and Sesame oil.

Another embodiment comprises cyclosporin A, T-Det O-6®, and Sesame oil.

Another embodiment comprises cyclosporin A, T-Det O-8®, and Sesame oil.

Another embodiment comprises cyclosporin A, T-Det O-9®, and Sesame oil.

Another embodiment comprises cyclosporin A, Tegin®, and Sesame oil.

Another embodiment comprises cyclosporin A, Tergitol 15-S-12®, and Sesame oil.

Another embodiment comprises cyclosporin A, Tergitol 15-S-15®, and Sesame oil.

Another embodiment comprises cyclosporin A, Tergitol 15-S-20®, and Sesame oil.

Another embodiment comprises cyclosporin A, Tergitol 15-S-20 (8AQ)®, and Sesame oil.

Another embodiment comprises cyclosporin A, Tergitol 15-S-3®, and Sesame oil.

Another embodiment comprises cyclosporin A, Tergitol 15-S-30®, and Sesame oil.

Another embodiment comprises cyclosporin A, Tergitol 15-S-40®, and Sesame oil.

Another embodiment comprises cyclosporin A, Tergitol 15-S-40 (70AQ)®, and Sesame oil.

Another embodiment comprises cyclosporin A, Tergitol 15-S-5®, and Sesame oil.

Another embodiment comprises cyclosporin A, Tergitol 15-S-7®, and Sesame oil.

Another embodiment comprises cyclosporin A, Tergitol 15-S-9®, and Sesame oil.

Another embodiment comprises cyclosporin A, Tergitol L-101®, and Sesame oil.

Another embodiment comprises cyclosporin A, Tergitol L-61®, and Sesame oil.

Another embodiment comprises cyclosporin A, Tergitol L-62®, and Sesame oil.

Another embodiment comprises cyclosporin A, Tergitol L-64®, and Sesame oil.

Another embodiment comprises cyclosporin A, Tergitol L-81®, and Sesame oil.

Another embodiment comprises cyclosporin A, Tergitol NP-10®, and Sesame oil.

Another embodiment comprises cyclosporin A, Tergitol NP-11®, and Sesame oil.

Another embodiment comprises cyclosporin A, Tergitol NP-12®, and Sesame oil.

Another embodiment comprises cyclosporin A, Tergitol NP-13®, and Sesame oil.

Another embodiment comprises cyclosporin A, Tergitol NP-15®, and Sesame oil.

Another embodiment comprises cyclosporin A, Tergitol NP-30®, and Sesame oil.

Another embodiment comprises cyclosporin A, Tergitol NP-30 (70 AQ)®, and Sesame oil.

Another embodiment comprises cyclosporin A, Tergitol NP-4®, and Sesame oil.

Another embodiment comprises cyclosporin A, Tergitol NP-40®, and Sesame oil.

Another embodiment comprises cyclosporin A, Tergitol NP-40 (70 AQ)®, and Sesame oil.

Another embodiment comprises cyclosporin A, Tergitol NP-50 (70 AQ)®, and Sesame oil.

Another embodiment comprises cyclosporin A, Tergitol NP-55 (70 AQ)®, and Sesame oil.

Another embodiment comprises cyclosporin A, Tergitol NP-6®, and Sesame oil.

Another embodiment comprises cyclosporin A, Tergitol NP-7®, and Sesame oil.

Another embodiment comprises cyclosporin A, Tergitol NP-70 (70 AQ)®, and Sesame oil.

Another embodiment comprises cyclosporin A, Tergitol NP-8®, and Sesame oil.

Another embodiment comprises cyclosporin A, Tergitol NP-9®, and Sesame oil.

Another embodiment comprises cyclosporin A, Tergitol NP-9.5®, and Sesame oil.

Another embodiment comprises cyclosporin A, Tergitol TMN-10®, and Sesame oil.

Another embodiment comprises cyclosporin A, Tergitol TMN-3®, and Sesame oil.

Another embodiment comprises cyclosporin A, Tergitol TMN-6®, and Sesame oil.

Another embodiment comprises cyclosporin A, Tergitol XD®, and Sesame oil.

Another embodiment comprises cyclosporin A, Tergitol XH®, and Sesame oil.

Another embodiment comprises cyclosporin A, Tergitol XJ®, and Sesame oil.

Another embodiment comprises cyclosporin A, Tetronic 1107®, and Sesame oil.

Another embodiment comprises cyclosporin A, Tetronic 1307®, and Sesame oil.

Another embodiment comprises cyclosporin A, Tetronic 150R10®, and Sesame oil.

Another embodiment comprises cyclosporin A, Tetronic 304®, and Sesame oil.

Another embodiment comprises cyclosporin A, Tetronic 701®, and Sesame oil.

Another embodiment comprises cyclosporin A, Tetronic 901®, and Sesame oil.

Another embodiment comprises cyclosporin A, Tetronic 904®, and Sesame oil.

Another embodiment comprises cyclosporin A, Tetronic 908®, and Sesame oil.

Another embodiment comprises cyclosporin A, Tetronic 90R4®, and Sesame oil.

Another embodiment comprises cyclosporin A, Tomadol 1-3®, and Sesame oil.

Another embodiment comprises cyclosporin A, Tomadol 1-5®, and Sesame oil.

Another embodiment comprises cyclosporin A, Tomadol 1-7®, and Sesame oil.

Another embodiment comprises cyclosporin A, Tomadol 23-1, and Sesame oil.

Another embodiment comprises cyclosporin A, Tomadol 23-3®, and Sesame oil.

Another embodiment comprises cyclosporin A, Tomadol 23-5®, and Sesame oil.

Another embodiment comprises cyclosporin A, Tomadol 23-6.5®, and Sesame oil.

Another embodiment comprises cyclosporin A, Tomadol 25-12®, and Sesame oil.

Another embodiment comprises cyclosporin A, Tomadol 25-3®, and Sesame oil.

Another embodiment comprises cyclosporin A, Tomadol 25-7®, and Sesame oil.

Another embodiment comprises cyclosporin A, Tomadol 25-9®, and Sesame oil.

Another embodiment comprises cyclosporin A, Tomadol 45-13®, and Sesame oil.

Another embodiment comprises cyclosporin A, Tomadol 45-2.5®, and Sesame oil.

Another embodiment comprises cyclosporin A, Tomadol 45-7®, and Sesame oil.

Another embodiment comprises cyclosporin A, Tomadol 91-2.5®, and Sesame oil.

Another embodiment comprises cyclosporin A, Tomadol 91-6®, and Sesame oil.

Another embodiment comprises cyclosporin A, Tomadol 91-8®, and Sesame oil.

Another embodiment comprises cyclosporin A, Triton X-100®, and Sesame oil.

Another embodiment comprises cyclosporin A, Triton X-102®, and Sesame oil.

Another embodiment comprises cyclosporin A, Triton X-114®, and Sesame oil.

Another embodiment comprises cyclosporin A, Triton X-15®, and Sesame oil.

Another embodiment comprises cyclosporin A, Triton X-165 (70%)®, and Sesame oil.

Another embodiment comprises cyclosporin A, Triton X-207®, and Sesame oil.

Another embodiment comprises cyclosporin A, Triton X-305 (70%)®, and Sesame oil.

Another embodiment comprises cyclosporin A, Triton X-35®, and Sesame oil.

Another embodiment comprises cyclosporin A, Triton X-405 (70%)®, and Sesame oil.

Another embodiment comprises cyclosporin A, Triton X-45®, and Sesame oil.

Another embodiment comprises cyclosporin A, Triton X-705 (70%)®, and Sesame oil.

Another embodiment comprises cyclosporin A, Volpo S-10®, and Sesame oil.

Another embodiment comprises cyclosporin A, Volpo S-2®, and Sesame oil.

Another embodiment comprises cyclosporin A, Volpo S-20®, and Sesame oil.

Another embodiment comprises cyclosporin A, Volpo-10®, and Sesame oil.

Another embodiment comprises cyclosporin A, Volpo-20®, and Sesame oil.

Another embodiment comprises cyclosporin A, Volpo-3®, and Sesame oil.

Another embodiment comprises cyclosporin A, Volpo-5®, and Sesame oil.

Another embodiment comprises cyclosporin A, Abex 2515®, and an oil having a Specific Gravity below 0.9

Another embodiment comprises cyclosporin A, Abex 2525/40®, and an oil having a Specific Gravity below 0.9

Another embodiment comprises cyclosporin A, Abex 2535®, and an oil having a Specific Gravity below 0.9

Another embodiment comprises cyclosporin A, Abex 2545®, and an oil having a Specific Gravity below 0.9

Another embodiment comprises cyclosporin A, Acconon E®, and an oil having a Specific Gravity below 0.9

Another embodiment comprises cyclosporin A, Alfonic 1216CO-9®, and an oil having a Specific Gravity below 0.9

Another embodiment comprises cyclosporin A, Alfonic 1412-30®, and an oil having a Specific Gravity below 0.9

Another embodiment comprises cyclosporin A, Alfonic TDA-12®, and an oil having a Specific Gravity below 0.9

Another embodiment comprises cyclosporin A, Alfonic TDA-3®, and an oil having a Specific Gravity below 0.9

Another embodiment comprises cyclosporin A, Alfonic TDA-4®, and an oil having a Specific Gravity below 0.9

Another embodiment comprises cyclosporin A, Alfonic TDA-6®, and an oil having a Specific Gravity below 0.9

Another embodiment comprises cyclosporin A, Alfonic TDA-7®, and an oil having a Specific Gravity below 0.9

Another embodiment comprises cyclosporin A, Alfonic TDA-8®, and an oil having a Specific Gravity below 0.9

Another embodiment comprises cyclosporin A, Alfonic TDA-9®, and an oil having a Specific Gravity below 0.9

Another embodiment comprises cyclosporin A, Alkamuls 400-DO®, and an oil having a Specific Gravity below 0.9

Another embodiment comprises cyclosporin A, Alkamuls 400-MO/E®, and an oil having a Specific Gravity below 0.9

Another embodiment comprises cyclosporin A, Ameroxol OE-10®, and an oil having a Specific Gravity below 0.9

Another embodiment comprises cyclosporin A, Ameroxol OE-2®, and an oil having a Specific Gravity below 0.9

Another embodiment comprises cyclosporin A, Ameroxol OE-20®, and an oil having a Specific Gravity below 0.9

Another embodiment comprises cyclosporin A, Armix 176®, and an oil having a Specific Gravity below 0.9

Another embodiment comprises cyclosporin A, Armix 180-C®, and an oil having a Specific Gravity below 0.9

Another embodiment comprises cyclosporin A, Armix 183®, and an oil having a Specific Gravity below 0.9

Another embodiment comprises cyclosporin A, Brij 35®, and an oil having a Specific Gravity below 0.9

Another embodiment comprises cyclosporin A, Brij 35 Liquid®, and an oil having a Specific Gravity below 0.9

Another embodiment comprises cyclosporin A, Brij 35 SP®, and an oil having a Specific Gravity below 0.9

Another embodiment comprises cyclosporin A, Canasol MJ 2109®, and an oil having a Specific Gravity below 0.9

Another embodiment comprises cyclosporin A, Canasol MJ 52®, and an oil having a Specific Gravity below 0.9

Another embodiment comprises cyclosporin A, Canasol R 3600®, and an oil having a Specific Gravity below 0.9

Another embodiment comprises cyclosporin A, Canasol R 3630®, and an oil having a Specific Gravity below 0.9

Another embodiment comprises cyclosporin A, Canasol R 4000 H®, and an oil having a Specific Gravity below 0.9

Another embodiment comprises cyclosporin A, Cedepal CA-210®, and an oil having a Specific Gravity below 0.9

Another embodiment comprises cyclosporin A, Cedepal CA-520®, and an oil having a Specific Gravity below 0.9

Another embodiment comprises cyclosporin A, Cedepal CA-630®, and an oil having a Specific Gravity below 0.9

Another embodiment comprises cyclosporin A, Cedepal CA-720®, and an oil having a Specific Gravity below 0.9

Another embodiment comprises cyclosporin A, Cedepal CA-890®, and an oil having a Specific Gravity below 0.9

Another embodiment comprises cyclosporin A, Cedepal CA-897®, and an oil having a Specific Gravity below 0.9

Another embodiment comprises cyclosporin A, Cedepal CO-210®, and an oil having a Specific Gravity below 0.9

Another embodiment comprises cyclosporin A, Cedepal CO-430®, and an oil having a Specific Gravity below 0.9

Another embodiment comprises cyclosporin A, Cedepal CO-530®, and an oil having a Specific Gravity below 0.9

Another embodiment comprises cyclosporin A, Cedepal CO-630®, and an oil having a Specific Gravity below 0.9

Another embodiment comprises cyclosporin A, Cedepal CO-710®, and an oil having a Specific Gravity below 0.9

Another embodiment comprises cyclosporin A, Cedepal CO-730®, and an oil having a Specific Gravity below 0.9

Another embodiment comprises cyclosporin A, Cedepal CO-880®, and an oil having a Specific Gravity below 0.9

Another embodiment comprises cyclosporin A, Cedepal CO-887®, and an oil having a Specific Gravity below 0.9

Another embodiment comprises cyclosporin A, Cedepal CO-890®, and an oil having a Specific Gravity below 0.9

Another embodiment comprises cyclosporin A, Cedepal CO-897®, and an oil having a Specific Gravity below 0.9

Another embodiment comprises cyclosporin A, Cedepal CO-970®, and an oil having a Specific Gravity below 0.9

Another embodiment comprises cyclosporin A, Cedepal CO-977®, and an oil having a Specific Gravity below 0.9

Another embodiment comprises cyclosporin A, Cedepal CO-990®, and an oil having a Specific Gravity below 0.9

Another embodiment comprises cyclosporin A, Cedepal CO-997®, and an oil having a Specific Gravity below 0.9

Another embodiment comprises cyclosporin A, Chemal BP 261®, and an oil having a Specific Gravity below 0.9

Another embodiment comprises cyclosporin A, Chemal BP 262®, and an oil having a Specific Gravity below 0.9

Another embodiment comprises cyclosporin A, Chemal BP 3172®, and an oil having a Specific Gravity below 0.9

Another embodiment comprises cyclosporin A, Chemal BP 3174®, and an oil having a Specific Gravity below 0.9

Another embodiment comprises cyclosporin A, Chemal DA-4®, and an oil having a Specific Gravity below 0.9

Another embodiment comprises cyclosporin A, Chemal DA-6®, and an oil having a Specific Gravity below 0.9

Another embodiment comprises cyclosporin A, Chemal DA-9®, and an oil having a Specific Gravity below 0.9

Another embodiment comprises cyclosporin A, Chemal LA-23®, and an oil having a Specific Gravity below 0.9

Another embodiment comprises cyclosporin A, Chemal LA-4®, and an oil having a Specific Gravity below 0.9

Another embodiment comprises cyclosporin A, Chemal LA-9®, and an oil having a Specific Gravity below 0.9

Another embodiment comprises cyclosporin A, Chemal OA-20G®, and an oil having a Specific Gravity below 0.9

Another embodiment comprises cyclosporin A, Chemal OA-5®, and an oil having a Specific Gravity below 0.9

Another embodiment comprises cyclosporin A, Chemal OA-9®, and an oil having a Specific Gravity below 0.9

Another embodiment comprises cyclosporin A, Chemal TDA-12®, and an oil having a Specific Gravity below 0.9

Another embodiment comprises cyclosporin A, Chemal TDA-15®, and an oil having a Specific Gravity below 0.9

Another embodiment comprises cyclosporin A, Chemal TDA-3®, and an oil having a Specific Gravity below 0.9

Another embodiment comprises cyclosporin A, Chemal TDA-6®, and an oil having a Specific Gravity below 0.9

Another embodiment comprises cyclosporin A, Chemal TDA-9®, and an oil having a Specific Gravity below 0.9

Another embodiment comprises cyclosporin A, Chemax CO-16®, and an oil having a Specific Gravity below 0.9

Another embodiment comprises cyclosporin A, Chemax CO-200/50®, and an oil having a Specific Gravity below 0.9

Another embodiment comprises cyclosporin A, Chemax CO-25®, and an oil having a Specific Gravity below 0.9

Another embodiment comprises cyclosporin A, Chemax CO-30®, and an oil having a Specific Gravity below 0.9

Another embodiment comprises cyclosporin A, Chemax CO-36®, and an oil having a Specific Gravity below 0.9

Another embodiment comprises cyclosporin A, Chemax CO-40®, and an oil having a Specific Gravity below 0.9

Another embodiment comprises cyclosporin A, Chemax CO-5®, and an oil having a Specific Gravity below 0.9

Another embodiment comprises cyclosporin A, Chemax CO-80®, and an oil having a Specific Gravity below 0.9

Another embodiment comprises cyclosporin A, Chemax DNP-150/50®, and an oil having a Specific Gravity below 0.9

Another embodiment comprises cyclosporin A, Chemax DNP-18®, and an oil having a Specific Gravity below 0.9

Another embodiment comprises cyclosporin A, Chemax DNP-8®, and an oil having a Specific Gravity below 0.9

Another embodiment comprises cyclosporin A, Chemax E 200 ML®, and an oil having a Specific Gravity below 0.9

Another embodiment comprises cyclosporin A, Chemax E 200 MO®, and an oil having a Specific Gravity below 0.9

Another embodiment comprises cyclosporin A, Chemax E 400 ML®, and an oil having a Specific Gravity below 0.9

Another embodiment comprises cyclosporin A, Chemax E 400 MO®, and an oil having a Specific Gravity below 0.9

Another embodiment comprises cyclosporin A, Chemax E 600 ML®, and an oil having a Specific Gravity below 0.9

Another embodiment comprises cyclosporin A, Chemax E 600 MO®, and an oil having a Specific Gravity below 0.9

Another embodiment comprises cyclosporin A, Chemax E1000 MO®, and an oil having a Specific Gravity below 0.9

Another embodiment comprises cyclosporin A, Chemax E1000 MS®, and an oil having a Specific Gravity below 0.9

Another embodiment comprises cyclosporin A, Chemax E200 MS®, and an oil having a Specific Gravity below 0.9

Another embodiment comprises cyclosporin A, Chemax E400 MS®, and an oil having a Specific Gravity below 0.9

Another embodiment comprises cyclosporin A, Chemax E600 MS®, and an oil having a Specific Gravity below 0.9

Another embodiment comprises cyclosporin A, Chemax HCO-16®, and an oil having a Specific Gravity below 0.9

Another embodiment comprises cyclosporin A, Chemax HCO-200/50®, and an oil having a Specific Gravity below 0.9

Another embodiment comprises cyclosporin A, Chemax HCO-25®, and an oil having a Specific Gravity below 0.9

Another embodiment comprises cyclosporin A, Chemax HCO-5®, and an oi! having a Specific Gravity below 0.9

Another embodiment comprises cyclosporin A, Chemax TO-16®, and an oil having a Specific Gravity below 0.9

Another embodiment comprises cyclosporin A, Chemax TO-8®, and an oil having a Specific Gravity below 0.9

Another embodiment comprises cyclosporin A, Colalube 3409®, and an oil having a Specific Gravity below 0.9

Another embodiment comprises cyclosporin A, Colalube 3411l, and an oil having a Specific Gravity below 0.9

Another embodiment comprises cyclosporin A, Colalube 3413®, and an oil having a Specific Gravity below 0.9

Another embodiment comprises cyclosporin A, Colalube 3414®, and an oil having a Specific Gravity below 0.9

Another embodiment comprises cyclosporin A, Colalube 3415®, and an oil having a Specific Gravity below 0.9

Another embodiment comprises cyclosporin A, Colalube 3416®, and an oil having a Specific Gravity below 0.9

Another embodiment comprises cyclosporin A, Colalube 3417®, and an oil having a Specific Gravity below 0.9

Another embodiment comprises cyclosporin A, Delonic NPE-100®, and an oil having a Specific Gravity below 0.9

Another embodiment comprises cyclosporin A, Delonic NPE-30®, and an oil having a Specific Gravity below 0.9

Another embodiment comprises cyclosporin A, Delonic NPE-40®, and an oil having a Specific Gravity below 0.9

Another embodiment comprises cyclosporin A, Delonic OPE-10®and an oil having a Specific Gravity below 0.9

Another embodiment comprises cyclosporin A, Delonic OPE-12®, and an oil having a Specific Gravity below 0.9

Another embodiment comprises cyclosporin A, Delonic OPE-30®, and an oil having a Specific Gravity below 0.9

Another embodiment comprises cyclosporin A, Delonic OPE-40®, and an oil having a Specific Gravity below 0.9

Another embodiment comprises cyclosporin A, Delonic OPE-5®, and an oil having a Specific Gravity below 0.9

Another embodiment comprises cyclosporin A, Delonic OPE-7.5®, and an oil having a Specific Gravity below 0.9

Another embodiment comprises cyclosporin A, DePeg 16-CO®, and an oil having a Specific Gravity below 0.9

Another embodiment comprises cyclosporin A, DePeg 25-CO®, and an oil having a Specific Gravity below 0.9

Another embodiment comprises cyclosporin A, DePeg 30-CO®, and an oil having a Specific Gravity below 0.9

Another embodiment comprises cyclosporin A, DePeg 40-CO®, and an oil having a Specific Gravity below 0.9

Another embodiment comprises cyclosporin A, DePeg 5-CO®, and an oil having a Specific Gravity below 0.9

Another embodiment comprises cyclosporin A, DePeg 80-CO®, and an oil having a Specific Gravity below 0.9

Another embodiment comprises cyclosporin A, DeSonic S-100®, and an oil having a Specific Gravity below 0.9

Another embodiment comprises cyclosporin A, DeSonic S-114®, and an oil having a Specific Gravity below 0.9

Another embodiment comprises cyclosporin A, DeSonic S-405®, and an oil having a Specific Gravity below 0.9

Another embodiment comprises cyclosporin A, DeSonic S-45®, and an oil having a Specific Gravity below 0.9

Another embodiment comprises cyclosporin A, DeSonic TDA-9®, and an oil having a Specific Gravity below 0.9

Another embodiment comprises cyclosporin A, DeThox Acid O-14®, and an oil having a Specific Gravity below 0.9

Another embodiment comprises cyclosporin A, DeThox Acid O-16®, and an oil having a Specific Gravity below 0.9

Another embodiment comprises cyclosporin A, DeThox Acid O-9®, and an oil having a Specific Gravity below 0.9

Another embodiment comprises cyclosporin A, DeThox Acid TO-14®, and an oil having a Specific Gravity below 0.9

Another embodiment comprises cyclosporin A, DeThox Acid TO-16.5®, and an oil having a Specific Gravity below 0.9

Another embodiment comprises cyclosporin A, DeThox Acid TO-8.5®, and an oil having a Specific Gravity below 0.9

Another embodiment comprises cyclosporin A, Dur-Em 114®, and an oil having a Specific Gravity below 0.9

Another embodiment comprises cyclosporin A, Dur-Em 117®, and an oil having a Specific Gravity below 0.9

Another embodiment comprises cyclosporin A, Dur-Em 204®, and an oil having a Specific Gravity below 0.9

Another embodiment comprises cyclosporin A, Dur-Em 207®, and an oil having a Specific Gravity below 0.9

Another embodiment comprises cyclosporin A, Dur-Em 207E®, and an oil having a Specific Gravity below 0.9

Another embodiment comprises cyclosporin A, Emerest 2704®, and an oil having a Specific Gravity below 0.9

Another embodiment comprises cyclosporin A, Emerest 2712®, and an oil having a Specific Gravity below 0.9

Another embodiment comprises cyclosporin A, Emerest 2715®, and an oil having a Specific Gravity below 0.9

Another embodiment comprises cyclosporin A, Ethal EH-2®, and an oil having a Specific Gravity below 0.9

Another embodiment comprises cyclosporin A, Ethal EH-5®, and an oil having a Specific Gravity below 0.9

Another embodiment comprises cyclosporin A, Ethal OA-23®, and an oil having a Specific Gravity below 0.9

Another embodiment comprises cyclosporin A, Ethosperse CA-20®, and an oil having a Specific Gravity below 0.9

Another embodiment comprises cyclosporin A, Ethosperse LA-12®, and an oil having a Specific Gravity below 0.9

Another embodiment comprises cyclosporin A, Ethosperse LA-23®, and an oil having a Specific Gravity below 0.9

Another embodiment comprises cyclosporin A, Ethosperse LA-4®, and an oil having a Specific Gravity below 0.9

Another embodiment comprises cyclosporin A, Ethox 3095®, and an oil having a Specific Gravity below 0.9

Another embodiment comprises cyclosporin A, Ethox CO-16®, and an oil having a Specific Gravity below 0.9

Another embodiment comprises cyclosporin A, Ethox CO-200®, and an oil having a Specific Gravity below 0.9

Another embodiment comprises cyclosporin A, Ethox CO-25®, and an oil having a Specific Gravity below 0.9

Another embodiment comprises cyclosporin A, Ethox CO-30®, and an oil having a Specific Gravity below 0.9

Another embodiment comprises cyclosporin A, Ethox CO-36®, and an oil having a Specific Gravity below 0.9

Another embodiment comprises cyclosporin A, Ethox CO-40®, and an oil having a Specific Gravity below 0.9

Another embodiment comprises cyclosporin A, Ethox CO-5®, and an oil having a Specific Gravity below 0.9

Another embodiment comprises cyclosporin A, Ethox DO-14®, and an oil having a Specific Gravity below 0.9

Another embodiment comprises cyclosporin A, Ethox DO-9®, and an oil having a Specific Gravity below 0.9

Another embodiment comprises cyclosporin A, Ethox MA-8®, and an oil having a Specific Gravity below 0.9

Another embodiment comprises cyclosporin A, Ethox MI-14®, and an oil having a Specific Gravity below 0.9

Another embodiment comprises cyclosporin A, Ethox ML-14®, and an oil having a Specific Gravity below 0.9

Another embodiment comprises cyclosporin A, Ethox ML-5®, and an oil having a Specific Gravity below 0.9

Another embodiment comprises cyclosporin A, Ethox MO-14®, and an oil having a Specific Gravity below 0.9

Another embodiment comprises cyclosporin A, Ethox MO-9®, and an oil having a Specific Gravity below 0.9

Another embodiment comprises cyclosporin A, Ethox MS-14®, and an oil having a Specific Gravity below 0.9

Another embodiment comprises cyclosporin A, Ethox MS-23®, and an oil having a Specific Gravity below 0.9

Another embodiment comprises cyclosporin A, Ethox MS-40®, and an oil having a Specific Gravity below 0.9

Another embodiment comprises cyclosporin A, Ethox MS-8®, and an oil having a Specific Gravity below 0.9

Another embodiment comprises cyclosporin A, Ethox TO-16®, and an oil having a Specific Gravity below 0.9

Another embodiment comprises cyclosporin A, Ethox TO-8®, and an oil having a Specific Gravity below 0.9

Another embodiment comprises cyclosporin A, Ethylan SN-120®, and an oil having a Specific Gravity below 0.9

Another embodiment comprises cyclosporin A, Ethylan SN-70®, and an oil having a Specific Gravity below 0.9

Another embodiment comprises cyclosporin A, Ethylan SN-90®, and an oil having a Specific Gravity below 0.9

Another embodiment comprises cyclosporin A, Flexricin 13®, and an oil having a Specific Gravity below 0.9

Another embodiment comprises cyclosporin A, Flexricin 15®, and an oil having a Specific Gravity below 0.9

Another embodiment comprises cyclosporin A, Genapol 1261®, and an oil having a Specific Gravity below 0.9

Another embodiment comprises cyclosporin A, Generol 122 E25®, and an oil having a Specific Gravity below 0.9

Another embodiment comprises cyclosporin A, Glicepol 160®, and an oil having a Specific Gravity below 0.9

Another embodiment comprises cyclosporin A, Glicepol 560®, and an oil having a Specific Gravity below 0.9

Another embodiment comprises cyclosporin A, Glicepol GMS 20®, and an oil having a Specific Gravity below 0.9

Another embodiment comprises cyclosporin A, Hetoxide BN-13®, and an oil having a Specific Gravity below 0.9

Another embodiment comprises cyclosporin A, Hetoxide DNP-4®, and an oil having a Specific Gravity below 0.9

Another embodiment comprises cyclosporin A, Hetoxide DNP-9.6®, and an oil having a Specific Gravity below 0.9

Another embodiment comprises cyclosporin A, Hetoxide G-26®, and an oil having a Specific Gravity below 0.9

Another embodiment comprises cyclosporin A, Hetoxide G-7®, and an oil having a Specific Gravity below 0.9

Another embodiment comprises cyclosporin A, Hetoxide GT-20®, and an oil having a Specific Gravity below 0.9

Another embodiment comprises cyclosporin A, Hetoxide HC-40®, and an oil having a Specific Gravity below 0.9

Another embodiment comprises cyclosporin A, Hetoxol C-24®, and an oil having a Specific Gravity below 0.9

Another embodiment comprises cyclosporin A, Hetoxol CA-10®, and an oil having a Specific Gravity below 0.9

Another embodiment comprises cyclosporin A, Hetoxol CA-2®, and an oil having a Specific Gravity below 0.9

Another embodiment comprises cyclosporin A, Hetoxol CA-20®, and an oil having a Specific Gravity below 0.9

Another embodiment comprises cyclosporin A, Hetoxol CAWS®, and an oil having a Specific Gravity below 0.9

Another embodiment comprises cyclosporin A, Hetoxol CD-3®, and an oil having a Specific Gravity below 0.9

Another embodiment comprises cyclosporin A, Hetoxol CD-4®, and an oil having a Specific Gravity below 0.9

Another embodiment comprises cyclosporin A, Hetoxol CS-15®, and an oil having a Specific Gravity below 0.9

Another embodiment comprises cyclosporin A, Hetoxol CS-20®, and an oil having a Specific Gravity below 0.9

Another embodiment comprises cyclosporin A, Hetoxol CS-20D®, and an oil having a Specific Gravity below 0.9

Another embodiment comprises cyclosporin A, Hetoxol CS-25®, and an oil having a Specific Gravity below 0.9

Another embodiment comprises cyclosporin A, Hetoxol CS-50®, and an oil having a Specific Gravity below 0.9

Another embodiment comprises cyclosporin A, Hetoxol CS-9®, and an oil having a Specific Gravity below 0.9

Another embodiment comprises cyclosporin A, Hetoxol CSA-15®, and an oil having a Specific Gravity below 0.9

Another embodiment comprises cyclosporin A, Hetoxol D®, and an oil having a Specific Gravity below 0.9

Another embodiment comprises cyclosporin A, Hetoxol G®, and an oil having a Specific Gravity below 0.9

Another embodiment comprises cyclosporin A, Hetoxol J®, and an oil having a Specific Gravity below 0.9

Another embodiment comprises cyclosporin A, Hetoxol L®, and an oil having a Specific Gravity below 0.9

Another embodiment comprises cyclosporin A, Hetoxol L-23®, and an oil having a Specific Gravity below 0.9

Another embodiment comprises cyclosporin A, Hetoxol L-4®, and an oil having a Specific Gravity below 0.9

Another embodiment comprises cyclosporin A, Hetoxol L-9®, and an oil having a Specific Gravity below 0.9

Another embodiment comprises cyclosporin A, Hetoxol LS-9®, and an oil having a Specific Gravity below 0.9

Another embodiment comprises cyclosporin A, Hetoxol M-3®, and an oil having a Specific Gravity below 0.9

Another embodiment comprises cyclosporin A, Hetoxol OA-10 Special®, and an oil having a Specific Gravity below 0.9

Another embodiment comprises cyclosporin A, Hetoxol OA-20 Special®, and an oil having a Specific Gravity below 0.9

Another embodiment comprises cyclosporin A, Hetoxol OA-3 Special®, and an oil having a Specific Gravity below 0.9

Another embodiment comprises cyclosporin A, Hetoxol OA-5 Special®, and an oil having a Specific Gravity below 0.9

Another embodiment comprises cyclosporin A, Hetoxol OL-10®and an oil having a Specific Gravity below 0.9

Another embodiment comprises cyclosporin A, Hetoxol OL-2®, and an oil having a Specific Gravity below 0.9

Another embodiment comprises cyclosporin A, Hetoxol OL-23®, and an oil having a Specific Gravity below 0.9

Another embodiment comprises cyclosporin A, Hetoxol OL-4®, and an oil having a Specific Gravity below 0.9

Another embodiment comprises cyclosporin A, Hetoxol OL-40®, and an oil having a Specific Gravity below 0.9

Another embodiment comprises cyclosporin A, Hetoxol OL-5®, and an oil having a Specific Gravity below 0.9

Another embodiment comprises cyclosporin A, Hetoxol PLA®, and an oil having a Specific Gravity below 0.9

Another embodiment comprises cyclosporin A, Hetoxol SP-15®, and an oil having a Specific Gravity below 0.9

Another embodiment comprises cyclosporin A, Hetoxol STA-10®, and an oil having a Specific Gravity below 0.9

Another embodiment comprises cyclosporin A, Hetoxol STA-2®, and an oil having a Specific Gravity below 0.9

Another embodiment comprises cyclosporin A, Hetoxol STA-30®, and an oil having a Specific Gravity below 0.9

Another embodiment comprises cyclosporin A, Hetoxol TD-12®, and an oil having a Specific Gravity below 0.9

Another embodiment comprises cyclosporin A, Hetoxol TD-18®, and an oil having a Specific Gravity below 0.9

Another embodiment comprises cyclosporin A, Hetoxol TD-3®, and an oil having a Specific Gravity below 0.9

Another embodiment comprises cyclosporin A, Hetoxol TD-6®, and an oil having a Specific Gravity below 0.9

Another embodiment comprises cyclosporin A, Hetoxol TD-9®, and an oil having a Specific Gravity below 0.9

Another embodiment comprises cyclosporin A, Hetoxol TDEP-15®, and an oil having a Specific Gravity below 0.9

Another embodiment comprises cyclosporin A, Hetoxol TDEP-63®, and an oil having a Specific Gravity below 0.9

Another embodiment comprises cyclosporin A, Iconol 24-12®, and an oil having a Specific Gravity below 0.9

Another embodiment comprises cyclosporin A, Iconol 24-23®, and an oil having a Specific Gravity below 0.9

Another embodiment comprises cyclosporin A, Iconol 24-4®, and an oil having a Specific Gravity below 0.9

Another embodiment comprises cyclosporin A, Iconol DA-4®, and an oil having a Specific Gravity below 0.9

Another embodiment comprises cyclosporin A, Iconol DA-6®, and an oil having a Specific Gravity below 0.9

Another embodiment comprises cyclosporin A, Iconol TDA-10®, and an oil having a Specific Gravity below 0.9

Another embodiment comprises cyclosporin A, Iconol TDA-3®, and an oil having a Specific Gravity below 0.9

Another embodiment comprises cyclosporin A, Iconol TDA-6®, and an oil having a Specific Gravity below 0.9

Another embodiment comprises cyclosporin A, Iconol TDA-8-90%®, and an oil having a Specific Gravity below 0.9

Another embodiment comprises cyclosporin A, Iconol TDA-9®, and an oil having a Specific Gravity below 0.9

Another embodiment comprises cyclosporin A, Igepal CA-210®, and an oil having a Specific Gravity below 0.9

Another embodiment comprises cyclosporin A, Igepal CA-407®, and an oil having a Specific Gravity below 0.9

Another embodiment comprises cyclosporin A, Igepal CA-420®, and an oil having a Specific Gravity below 0.9

Another embodiment comprises cyclosporin A, Igepal CA-520®, and an oil having a Specific Gravity below 0.9

Another embodiment comprises cyclosporin A, Igepal CA-620®, and an oil having a Specific Gravity below 0.9

Another embodiment comprises cyclosporin A, Igepal CA-630®, and an oil having a Specific Gravity below 0.9

Another embodiment comprises cyclosporin A, Igepal CA-720®, and an oil having a Specific Gravity below 0.9

Another embodiment comprises cyclosporin A, lgepal CA-877®, and an oil having a Specific Gravity below 0.9

Another embodiment comprises cyclosporin A, Igepal CA-887®, and an oil having a Specific Gravity below 0.9

Another embodiment comprises cyclosporin A, lgepal CA-897®, and an oil having a Specific Gravity below 0.9

Another embodiment comprises cyclosporin A, Igepal CO-210®, and an oil having a Specific Gravity below 0.9

Another embodiment comprises cyclosporin A, Igepal CO-430®, and an oil having a Specific Gravity below 0.9

Another embodiment comprises cyclosporin A, Igepal CO-520®, and an oil having a Specific Gravity below 0.9

Another embodiment comprises cyclosporin A, Igepal CO-530®, and an oil having a Specific Gravity below 0.9

Another embodiment comprises cyclosporin A, Igepal CO-610®, and an oil having a Specific Gravity below 0.9

Another embodiment comprises cyclosporin A, Igepal CO-630®, and an oil having a Specific Gravity below 0.9

Another embodiment comprises cyclosporin A, Igepal CO-630 Special®, and an oil having a Specific Gravity below 0.9

Another embodiment comprises cyclosporin A, Igepal CO-660®, and an oil having a Specific Gravity below 0.9

Another embodiment comprises cyclosporin A, Igepal CO-710®, and an oil having a Specific Gravity below 0.9

Another embodiment comprises cyclosporin A, Igepal CO-720®, and an oil having a Specific Gravity below 0.9

Another embodiment comprises cyclosporin A, lgepal CO-730®, and an oil having a Specific Gravity below 0.9

Another embodiment comprises cyclosporin A, Igepal CO-738®, and an oil having a Specific Gravity below 0.9

Another embodiment comprises cyclosporin A, Igepal CO-850®, and an oil having a Specific Gravity below 0.9

Another embodiment comprises cyclosporin A, lgepal CO-880®, and an oil having a Specific Gravity below 0.9

Another embodiment comprises cyclosporin A, Igepal CO-887®, and an oil having a Specific Gravity below 0.9

Another embodiment comprises cyclosporin A, Igepal CO-890®, and an oil having a Specific Gravity below 0.9

Another embodiment comprises cyclosporin A, Igepal CO-897®, and an oil having a Specific Gravity below 0.9

Another embodiment comprises cyclosporin A, Igepal CO-970®, and an oil having a Specific Gravity below 0.9

Another embodiment comprises cyclosporin A, lgepal CO-977®, and an oil having a Specific Gravity below 0.9

Another embodiment comprises cyclosporin A, lgepal CO-987®, and an oil having a Specific Gravity below 0.9

Another embodiment comprises cyclosporin A, lgepal CO-990 FLK®, and an oil having a Specific Gravity below 0.9

Another embodiment comprises cyclosporin A, lgepal CO-997®, and an oil having a Specific Gravity below 0.9

Another embodiment comprises cyclosporin A, lgepal DM-430®, and an oil having a Specific Gravity below 0.9

Another embodiment comprises cyclosporin A, Igepal DM-530®, and an oil having a Specific Gravity below 0.9

Another embodiment comprises cyclosporin A, Igepal DM-710®, and an oil having a Specific Gravity below 0.9

Another embodiment comprises cyclosporin A, Igepal DM-730®, and an oil having a Specific Gravity below 0.9

Another embodiment comprises cyclosporin A, Igepal DM-880®, and an oil having a Specific Gravity below 0.9

Another embodiment comprises cyclosporin A, lgepal DM-970 FLK®, and an oil having a Specific Gravity below 0.9

Another embodiment comprises cyclosporin A, Igepal OD-410®, and an oil having a Specific Gravity below 0.9

Another embodiment comprises cyclosporin A, Igepal RC-520®, and an oil having a Specific Gravity below 0.9

Another embodiment comprises cyclosporin A, Igepal RC-620®, and an oil having a Specific Gravity below 0.9

Another embodiment comprises cyclosporin A, Igepal RC-630®, and an oil having a Specific Gravity below 0.9

Another embodiment comprises cyclosporin A, Lexemul CS-20®, and an oil having a Specific Gravity below 0.9

Another embodiment comprises cyclosporin A, Lipo GMS 450®, and an oil having a Specific Gravity below 0.9

Another embodiment comprises cyclosporin A, Lipo Polyglycol 1000®, and an oil having a Specific Gravity below 0.9

Another embodiment comprises cyclosporin A, Lipo Polyglycol 200®, and an oil having a Specific Gravity below 0.9

Another embodiment comprises cyclosporin A, Lipo Polyglycol 300®, and an oil having a Specific Gravity below 0.9

Another embodiment comprises cyclosporin A, Lipo Polyglycol 3350®, and an oil having a Specific Gravity below 0.9

Another embodiment comprises cyclosporin A, Lipo Polyglycol 400®, and an oil having a Specific Gravity below 0.9

Another embodiment comprises cyclosporin A, Lipo Polyglycol 600®, and an oil having a Specific Gravity below 0.9

Another embodiment comprises cyclosporin A, Lipocol C-10®, and an oil having a Specific Gravity below 0.9

Another embodiment comprises cyclosporin A, Lipocol C-2®, and an oil having a Specific Gravity below 0.9

Another embodiment comprises cyclosporin A, Lipocol C-20®, and an oil having a Specific Gravity below 0.9

Another embodiment comprises cyclosporin A, Lipocol L-12®, and an oil having a Specific Gravity below 0.9

Another embodiment comprises cyclosporin A, Lipocol L-23®, and an oil having a Specific Gravity below 0.9

Another embodiment comprises cyclosporin A, Lipocol L-4®, and an oil having a Specific Gravity below 0.9

Another embodiment comprises cyclosporin A, Lipocol O-10®, and an oil having a Specific Gravity below 0.9

Another embodiment comprises cyclosporin A, Lipocol O-2®, and an oil having a Specific Gravity below 0.9

Another embodiment comprises cyclosporin A, Lipocol O-20®, and an oil having a Specific Gravity below 0.9

Another embodiment comprises cyclosporin A, Lipocol O-5®, and an oil having a Specific Gravity below 0.9

Another embodiment comprises cyclosporin A, Lipocol S-10®, and an oil having a Specific Gravity below 0.9

Another embodiment comprises cyclosporin A, Lipocol S-2®, and an oil having a Specific Gravity below 0.9

Another embodiment comprises cyclosporin A, Lipocol S-20®, and an oil having a Specific Gravity below 0.9

Another embodiment comprises cyclosporin A, Lipocol SC-15®, and an oil having a Specific Gravity below 0.9

Another embodiment comprises cyclosporin A, Lipocol SC-20®, and an oil having a Specific Gravity below 0.9

Another embodiment comprises cyclosporin A, Lipocol SC-4®, and an oil having a Specific Gravity below 0.9

Another embodiment comprises cyclosporin A, Lipocol TD-12®, and an oil having a Specific Gravity below 0.9

Another embodiment comprises cyclosporin A, Lumulse 40-O K®, and an oil having a Specific Gravity below 0.9

Another embodiment comprises cyclosporin A, Lumulse 42-O K®, and an oil having a Specific Gravity below 0.9

Another embodiment comprises cyclosporin A, Lumulse GMT-40®, and an oil having a Specific Gravity below 0.9

Another embodiment comprises cyclosporin A, Lumulse PEG 1450®, and an oil having a Specific Gravity below 0.9

Another embodiment comprises cyclosporin A, Lumulse PEG 1450 NF®, and an oil having a Specific Gravity below 0.9

Another embodiment comprises cyclosporin A, Lumulse PEG 8000®, and an oil having a Specific Gravity below 0.9

Another embodiment comprises cyclosporin A, Lumulse PGO®, and an oil having a Specific Gravity below 0.9

Another embodiment comprises cyclosporin A, Lumulse POE (2) Oleyl Amine®, and an oil having a Specific Gravity below 0.9

Another embodiment comprises cyclosporin A, Magrabar PGO-104®, and an oil having a Specific Gravity below 0.9

Another embodiment comprises cyclosporin A, Mapeg 200 ML®, and an oil having a Specific Gravity below 0.9

Another embodiment comprises cyclosporin A, Mapeg 400 MO®, and an oil having a Specific Gravity below 0.9

Another embodiment comprises cyclosporin A, Mapeg 400 MOT®, and an oil having a Specific Gravity below 0.9

Another embodiment comprises cyclosporin A, Mapeg 400DO®, and an oil having a Specific Gravity below 0.9

Another embodiment comprises cyclosporin A, Mapeg 400DOT®, and an oil having a Specific Gravity below 0.9

Another embodiment comprises cyclosporin A, Mapeg 600DOT®, and an oil having a Specific Gravity below 0.9

Another embodiment comprises cyclosporin A, Marlox B 24/80®, and an oil having a Specific Gravity below 0.9

Another embodiment comprises cyclosporin A, Marlox FK 86®, and an oil having a Specific Gravity below 0.9

Another embodiment comprises cyclosporin A, Marlox MO 154®, and an oil having a Specific Gravity below 0.9

Another embodiment comprises cyclosporin A, Mazox CDA CG®, and an oil having a Specific Gravity below 0.9

Another embodiment comprises cyclosporin A, Monoglycerides Glyceryl Monestearate®, and an oil having a Specific Gravity below 0.9

Another embodiment comprises cyclosporin A, Myrj 45®, and an oil having a Specific Gravity below 0.9

Another embodiment comprises cyclosporin A, Myrj 52®, and an oil having a Specific Gravity below 0.9

Another embodiment comprises cyclosporin A, Myrj 52S®, and an oil having a Specific Gravity below 0.9

Another embodiment comprises cyclosporin A, Myrj 53®, and an oil having a Specific Gravity below 0.9

Another embodiment comprises cyclosporin A, Myrj 59®, and an oil having a Specific Gravity below 0.9

Another embodiment comprises cyclosporin A, Norfox NP-12®, and an oil having a Specific Gravity below 0.9

Another embodiment comprises cyclosporin A, Norfox NP-4®, and an oil having a Specific Gravity below 0.9

Another embodiment comprises cyclosporin A, Norfox NP-6®, and an oil having a Specific Gravity below 0.9

Another embodiment comprises cyclosporin A, Norfox NP-887®, and an oil having a Specific Gravity below 0.9

Another embodiment comprises cyclosporin A, Norfox NP-9®, and an oil having a Specific Gravity below 0.9

Another embodiment comprises cyclosporin A, Norfox NP-977®, and an oil having a Specific Gravity below 0.9

Another embodiment comprises cyclosporin A, Norfox OP-100®, and an oil having a Specific Gravity below 0.9

Another embodiment comprises cyclosporin A, Norfox OP-102®, and an oil having a Specific Gravity below 0.9

Another embodiment comprises cyclosporin A, Norfox OP-114®, and an oil having a Specific Gravity below 0.9

Another embodiment comprises cyclosporin A, Norfox OP-45®, and an oil having a Specific Gravity below 0.9

Another embodiment comprises cyclosporin A, Pegosperse 100-L®, and an oil having a Specific Gravity below 0.9

Another embodiment comprises cyclosporin A, Pegosperse 100-O®, and an oil having a Specific Gravity below 0.9

Another embodiment comprises cyclosporin A, Pegosperse 100-S®, and an oil having a Specific Gravity below 0.9

Another embodiment comprises cyclosporin A, Pegosperse 1500-MS®, and an oil having a Specific Gravity below 0.9

Another embodiment comprises cyclosporin A, Pegosperse 1750-MS®, and an oil having a Specific Gravity below 0.9

Another embodiment comprises cyclosporin A, Pegosperse 200 DL®, and an oil having a Specific Gravity below 0.9

Another embodiment comprises cyclosporin A, Pegosperse 200-ML®, and an oil having a Specific Gravity below 0.9

Another embodiment comprises cyclosporin A, Pegosperse 400-DL®, and an oil having a Specific Gravity below 0.9

Another embodiment comprises cyclosporin A, Pegosperse 400-DO®, and an oil having a Specific Gravity below 0.9

Another embodiment comprises cyclosporin A, Pegosperse 400-DS®, and an oil having a Specific Gravity below 0.9

Another embodiment comprises cyclosporin A, Pegosperse 400-ML®, and an oil having a Specific Gravity below 0.9

Another embodiment comprises cyclosporin A, Pegosperse 400-MO®, and an oil having a Specific Gravity below 0.9

Another embodiment comprises cyclosporin A, Pegosperse 400-MOT®, and an oil having a Specific Gravity below 0.9

Another embodiment comprises cyclosporin A, Pegosperse 400-MS®, and an oil having a Specific Gravity below 0.9

Another embodiment comprises cyclosporin A, Pegosperse 50-DS®, and an oil having a Specific Gravity below 0.9

Another embodiment comprises cyclosporin A, Pegosperse 50-MS®, and an oil having a Specific Gravity below 0.9

Another embodiment comprises cyclosporin A, Pegosperse 600-ML®, and an oil having a Specific Gravity below 0.9

Another embodiment comprises cyclosporin A, Pegosperse 600-MS®, and an oil having a Specific Gravity below 0.9

Another embodiment comprises cyclosporin A, Plurafac A-38®, and an oil having a Specific Gravity below 0.9

Another embodiment comprises cyclosporin A, Plurafac B-25-5®, and an oil having a Specific Gravity below 0.9

Another embodiment comprises cyclosporin A, Plurafac B-26®, and an oil having a Specific Gravity below 0.9

Another embodiment comprises cyclosporin A, Plurafac D-25®, and an oil having a Specific Gravity below 0.9

Another embodiment comprises cyclosporin A, Plurafac LF 131®, and an oil having a Specific Gravity below 0.9

Another embodiment comprises cyclosporin A, Plurafac LF 4030®, and an oil having a Specific Gravity below 0.9

Another embodiment comprises cyclosporin A, Plurafac LF 7000®, and an oil having a Specific Gravity below 0.9

Another embodiment comprises cyclosporin A, Plurafac RA-20®, and an oil having a Specific Gravity below 0.9

Another embodiment comprises cyclosporin A, Plurafac RA-30®, and an oil having a Specific Gravity below 0.9

Another embodiment comprises cyclosporin A, Plurafac RA-40®, and an oil having a Specific Gravity below 0.9

Another embodiment comprises cyclosporin A, Pluronic 10R5®, and an oil having a Specific Gravity below 0.9

Another embodiment comprises cyclosporin A, Pluronic 17R2®, and an oil having a Specific Gravity below 0.9

Another embodiment comprises cyclosporin A, Pluronic 17R4®, and an oil having a Specific Gravity below 0.9

Another embodiment comprises cyclosporin A, Pluronic 25R2®, and an oil having a Specific Gravity below 0.9

Another embodiment comprises cyclosporin A, Pluronic 25R4®, and an oil having a Specific Gravity below 0.9

Another embodiment comprises cyclosporin A, Pluronic 31R1®, and an oil having a Specific Gravity below 0.9

Another embodiment comprises cyclosporin A, Pluronic F108®, and an oil having a Specific Gravity below 0.9

Another embodiment comprises cyclosporin A, Pluronic F127®, and an oil having a Specific Gravity below 0.9

Another embodiment comprises cyclosporin A, Pluronic F38®, and an oil having a Specific Gravity below 0.9

Another embodiment comprises cyclosporin A, Pluronic F68®, and an oil having a Specific Gravity below 0.9

Another embodiment comprises cyclosporin A, Pluronic F68LF®, and an oil having a Specific Gravity below 0.9

Another embodiment comprises cyclosporin A, Pluronic F77®, and an oil having a Specific Gravity below 0.9

Another embodiment comprises cyclosporin A, Pluronic F87®, and an oil having a Specific Gravity below 0.9

Another embodiment comprises cyclosporin A, Pluronic F88®, and an oil having a Specific Gravity below 0.9

Another embodiment comprises cyclosporin A, Pluronic F98®, and an oil having a Specific Gravity below 0.9

Another embodiment comprises cyclosporin A, Pluronic L10®, and an oil having a Specific Gravity below 0.9

Another embodiment comprises cyclosporin A, Pluronic L101®, and an oil having a Specific Gravity below 0.9

Another embodiment comprises cyclosporin A, Pluronic L121®, and an oil having a Specific Gravity below 0.9

Another embodiment comprises cyclosporin A, Pluronic L31®, and an oil having a Specific Gravity below 0.9

Another embodiment comprises cyclosporin A, Pluronic L35®, and an oil having a Specific Gravity below 0.9

Another embodiment comprises cyclosporin A, Pluronic L43®, and an oil having a Specific Gravity below 0.9

Another embodiment comprises cyclosporin A, Pluronic L44®, and an oil having a Specific Gravity below 0.9

Another embodiment comprises cyclosporin A, Pluronic L61®, and an oil having a Specific Gravity below 0.9

Another embodiment comprises cyclosporin A, Pluronic L62®, and an oil having a Specific Gravity below 0.9

Another embodiment comprises cyclosporin A, Pluronic L62D®, and an oil having a Specific Gravity below 0.9

Another embodiment comprises cyclosporin A, Pluronic L62LF®, and an oil having a Specific Gravity below 0.9

Another embodiment comprises cyclosporin A, Pluronic L64®, and an oil having a Specific Gravity below 0.9

Another embodiment comprises cyclosporin A, Pluronic L81®, and an oil having a Specific Gravity below 0.9

Another embodiment comprises cyclosporin A, Pluronic L92®, and an oil having a Specific Gravity below 0.9

Another embodiment comprises cyclosporin A, Pluronic P103®, and an oil having a Specific Gravity below 0.9

Another embodiment comprises cyclosporin A, Pluronic P104®, and an oil having a Specific Gravity below 0.9

Another embodiment comprises cyclosporin A, Pluronic P105®, and an oil having a Specific Gravity below 0.9

Another embodiment comprises cyclosporin A, Pluronic P123®, and an oil having a Specific Gravity below 0.9

Another embodiment comprises cyclosporin A, Pluronic P65®, and an oil having a Specific Gravity below 0.9

Another embodiment comprises cyclosporin A, Pluronic P84®, and an oil having a Specific Gravity below 0.9

Another embodiment comprises cyclosporin A, Pluronic P85®, and an oil having a Specific Gravity below 0.9

Another embodiment comprises cyclosporin A, Polyaldo 10-2-P®, and an oil having a Specific Gravity below 0.9

Another embodiment comprises cyclosporin A, Polyaldo DGDO®, and an oil having a Specific Gravity below 0.9

Another embodiment comprises cyclosporin A, Polyaldo HGDS®, and an oil having a Specific Gravity below 0.9

Another embodiment comprises cyclosporin A, Polyaldo TGMS®, and an oil having a Specific Gravity below 0.9

Another embodiment comprises cyclosporin A, Protegin V®, and an oil having a Specific Gravity below 0.9

Another embodiment comprises cyclosporin A, Protegin W®, and an oil having a Specific Gravity below 0.9

Another embodiment comprises cyclosporin A, Protegin WX®, and an oil having a Specific Gravity below 0.9

Another embodiment comprises cyclosporin A, Protegin XV®, and an oil having a Specific Gravity below 0.9

Another embodiment comprises cyclosporin A, Rhodasurf BC-420®, and an oil having a Specific Gravity below 0.9

Another embodiment comprises cyclosporin A, Rhodasurf BC-610®, and an oil having a Specific Gravity below 0.9

Another embodiment comprises cyclosporin A, Rhodasurf BC-630®, and an oil having a Specific Gravity below 0.9

Another embodiment comprises cyclosporin A, Rhodasurf BC-720®, and an oil having a Specific Gravity below 0.9

Another embodiment comprises cyclosporin A, Rhodasurf BC-840®, and an oil having a Specific Gravity below 0.9

Another embodiment comprises cyclosporin A, Rhodasurf L 1®, and an oil having a Specific Gravity below 0.9

Another embodiment comprises cyclosporin A, Rhodasurf L 12®, and an oil having a Specific Gravity below 0.9

Another embodiment comprises cyclosporin A, Rhodasurf L 2®, and an oil having a Specific Gravity below 0.9

Another embodiment comprises cyclosporin A, Rhodasurf L 20®, and an oil having a Specific Gravity below 0.9

Another embodiment comprises cyclosporin A, Rhodasurf L 3®, and an oil having a Specific Gravity below 0.9

Another embodiment comprises cyclosporin A, Rhodasurf L 30®, and an oil having a Specific Gravity below 0.9

Another embodiment comprises cyclosporin A, Rhodasurf L 4®, and an oil having a Specific Gravity below 0.9

Another embodiment comprises cyclosporin A, Rhodasurf L 50®, and an oil having a Specific Gravity below 0.9

Another embodiment comprises cyclosporin A, Rhodasurf L 7/90®, and an oil having a Specific Gravity below 0.9

Another embodiment comprises cyclosporin A, Rhodasurf LA-3®, and an oil having a Specific Gravity below 0.9

Another embodiment comprises cyclosporin A, Rhodasurf LA-7®, and an oil having a Specific Gravity below 0.9

Another embodiment comprises cyclosporin A, Rhodasurf LAN-23®, and an oil having a Specific Gravity below 0.9

Another embodiment comprises cyclosporin A, Rhodasurf ON-870®, and an oil having a Specific Gravity below 0.9

Another embodiment comprises cyclosporin A, Rhodasurf ON-877®, and an oil having a Specific Gravity below 0.9

Another embodiment comprises cyclosporin A, Rhodasurf TDA-8/5®, and an oil having a Specific Gravity below 0.9

Another embodiment comprises cyclosporin A, Ritapro 100®, and an oil having a Specific Gravity below 0.9

Another embodiment comprises cyclosporin A, Ritapro 165®, and an oil having a Specific Gravity below 0.9

Another embodiment comprises cyclosporin A, Ritapro 200®, and an oil having a Specific Gravity below 0.9

Another embodiment comprises cyclosporin A, Ritapro 300®, and an oil having a Specific Gravity below 0.9

Another embodiment comprises cyclosporin A, Ritoleth 10®, and an oil having a Specific Gravity below 0.9

Another embodiment comprises cyclosporin A, Ritoleth 2®, and an oil having a Specific Gravity below 0.9

Another embodiment comprises cyclosporin A, Ritoleth 20®, and an oil having a Specific Gravity below 0.9

Another embodiment comprises cyclosporin A, Ritoleth 5®, and an oil having a Specific Gravity below 0.9

Another embodiment comprises cyclosporin A, Santone 10-10-0®, and an oil having a Specific Gravity below 0.9

Another embodiment comprises cyclosporin A, Santone 3-1-S XTR®, and an oil having a Specific Gravity below 0.9

Another embodiment comprises cyclosporin A, Santone 3-1 -SH®and an oil having a Specific Gravity below 0.9

Another embodiment comprises cyclosporin A, Santone 8-1-O®, and an oil having a Specific Gravity below 0.9

Another embodiment comprises cyclosporin A, Solulan 16®, and an oil having a Specific Gravity below 0.9

Another embodiment comprises cyclosporin A, Solulan 75®, and an oil having a Specific Gravity below 0.9

Another embodiment comprises cyclosporin A, Solulan C-24®, and an oil having a Specific Gravity below 0.9

Another embodiment comprises cyclosporin A, Solulan L-575®, and an oil having a Specific Gravity below 0.9

Another embodiment comprises cyclosporin A, Standamox CAW®, and an oil having a Specific Gravity below 0.9

Another embodiment comprises cyclosporin A, Surfine AZI-A®, and an oil having a Specific Gravity below 0.9

Another embodiment comprises cyclosporin A, Surfonic CO-15®, and an oil having a Specific Gravity below 0.9

Another embodiment comprises cyclosporin A, Surfonic CO-25®, and an oil having a Specific Gravity below 0.9

Another embodiment comprises cyclosporin A, Surfonic CO-30®, and an oil having a Specific Gravity below 0.9

Another embodiment comprises cyclosporin A, Surfonic CO-36®, and an oil having a Specific Gravity below 0.9

Another embodiment comprises cyclosporin A, Surfonic CO-42®, and an oil having a Specific Gravity below 0.9

Another embodiment comprises cyclosporin A, Surfonic E400-MO®, and an oil having a Specific Gravity below 0.9

Another embodiment comprises cyclosporin A, Surfonic L12-3®, and an oil having a Specific Gravity below 0.9

Another embodiment comprises cyclosporin A, Surfonic L12-6®, and an oil having a Specific Gravity below 0.9

Another embodiment comprises cyclosporin A, Surfonic L12-8®, and an oil having a Specific Gravity below 0.9

Another embodiment comprises cyclosporin A, Surfonic L24-12®, and an oil having a Specific Gravity below 0.9

Another embodiment comprises cyclosporin A, Surfonic L24-2®, and an oil having a Specific Gravity below 0.9

Another embodiment comprises cyclosporin A, Surfonic L24-22®, and an oil having a Specific Gravity below 0.9

Another embodiment comprises cyclosporin A, Surfonic L24-3®, and an oil having a Specific Gravity below 0.9

Another embodiment comprises cyclosporin A, Surfonic L24-4®, and an oil having a Specific Gravity below 0.9

Another embodiment comprises cyclosporin A, Surfonic L24-5®, and an oil having a Specific Gravity below 0.9

Another embodiment comprises cyclosporin A, Surfonic L24-7®, and an oil having a Specific Gravity below 0.9

Another embodiment comprises cyclosporin A, Surfonic L24-7.1®, and an oil having a Specific Gravity below 0.9

Another embodiment comprises cyclosporin A, Surfonic L24-9®, and an oil having a Specific Gravity below 0.9

Another embodiment comprises cyclosporin A, Surfonic LF-17®, and an oil having a Specific Gravity below 0.9

Another embodiment comprises cyclosporin A, Surfonic LF-18®, and an oil having a Specific Gravity below 0.9

Another embodiment comprises cyclosporin A, Surfonic LF-37®, and an oil having a Specific Gravity below 0.9

Another embodiment comprises cyclosporin A, Surfonic LF-41®, and an oil having a Specific Gravity below 0.9

Another embodiment comprises cyclosporin A, Surfonic LF-42®, and an oil having a Specific Gravity below 0.9

Another embodiment comprises cyclosporin A, Surfonic LF-47®, and an oil having a Specific Gravity below 0.9

Another embodiment comprises cyclosporin A, Surfonic LF-68®, and an oil having a Specific Gravity below 0.9

Another embodiment comprises cyclosporin A, Surfonic N-10®, and an oil having a Specific Gravity below 0.9

Another embodiment comprises cyclosporin A, Surfonic N-100®, and an oil having a Specific Gravity below 0.9

Another embodiment comprises cyclosporin A, Surfonic N-102®, and an oil having a Specific Gravity below 0.9

Another embodiment comprises cyclosporin A, Surfonic N-120®, and an oil having a Specific Gravity below 0.9

Another embodiment comprises cyclosporin A, Surfonic N-150®, and an oil having a Specific Gravity below 0.9

Another embodiment comprises cyclosporin A, Surfonic N-200®, and an oil having a Specific Gravity below 0.9

Another embodiment comprises cyclosporin A, Surfonic N-300®, and an oil having a Specific Gravity below 0.9

Another embodiment comprises cyclosporin A, Surfonic N-31.5®, and an oil having a Specific Gravity below 0.9

Another embodiment comprises cyclosporin A, Surfonic N-40®, and an oil having a Specific Gravity below 0.9

Another embodiment comprises cyclosporin A, Surfonic N-400®, and an oil having a Specific Gravity below 0.9

Another embodiment comprises cyclosporin A, Surfonic N-60®, and an oil having a Specific Gravity below 0.9

Another embodiment comprises cyclosporin A, Surfonic N-85®, and an oil having a Specific Gravity below 0.9

Another embodiment comprises cyclosporin A, Surfonic N-95®, and an oil having a Specific Gravity below 0.9

Another embodiment comprises cyclosporin A, Surfonic NB-18®, and an oil having a Specific Gravity below 0.9

Another embodiment comprises cyclosporin A, Surfonic NB-23®, and an oil having a Specific Gravity below 0.9

Another embodiment comprises cyclosporin A, Surfonic NB-25®, and an oil having a Specific Gravity below 0.9

Another embodiment comprises cyclosporin A, Surfonic NB-307®, and an oil having a Specific Gravity below 0.9

Another embodiment comprises cyclosporin A, Surfonic NB-31®, and an oil having a Specific Gravity below 0.9

Another embodiment comprises cyclosporin A, Surfonic NB-41®, and an oil having a Specific Gravity below 0.9

Another embodiment comprises cyclosporin A, Surfonic NB-43®, and an oil having a Specific Gravity below 0.9

Another embodiment comprises cyclosporin A, Surfonic OP-100®, and an oil having a Specific Gravity below 0.9

Another embodiment comprises cyclosporin A, Surfonic OP-120®, and an oil having a Specific Gravity below 0.9

Another embodiment comprises cyclosporin A, Surfonic OP-15®, and an oil having a Specific Gravity below 0.9

Another embodiment comprises cyclosporin A, Surfonic OP-35®, and an oil having a Specific Gravity below 0.9

Another embodiment comprises cyclosporin A, Surfonic OP-70®, and an oil having a Specific Gravity below 0.9

Another embodiment comprises cyclosporin A, Surfonic P1®, and an oil having a Specific Gravity below 0.9

Another embodiment comprises cyclosporin A, Surfonic P3®, and an oil having a Specific Gravity below 0.9

Another embodiment comprises cyclosporin A, Surfonic P5®, and an oil having a Specific Gravity below 0.9

Another embodiment comprises cyclosporin A, Surfonic P6®, and an oil having a Specific Gravity below 0.9

Another embodiment comprises cyclosporin A, Surfonic POA-17R2®, and an oil having a Specific Gravity below 0.9

Another embodiment comprises cyclosporin A, Surfonic POA-25R2®, and an oil having a Specific Gravity below 0.9

Another embodiment comprises cyclosporin A, Surfonic TDA-11®, and an oil having a Specific Gravity below 0.9

Another embodiment comprises cyclosporin A, Surfonic TDA-3B®, and an oil having a Specific Gravity below 0.9

Another embodiment comprises cyclosporin A, Surfonic TDA-6®, and an oil having a Specific Gravity below 0.9

Another embodiment comprises cyclosporin A, Surfonic TDA-6/88®, and an oil having a Specific Gravity below 0.9

Another embodiment comprises cyclosporin A, Surfonic TDA-8®, and an oil having a Specific Gravity below 0.9

Another embodiment comprises cyclosporin A, Surfonic TDA-8/90®, and an oil having a Specific Gravity below 0.9

Another embodiment comprises cyclosporin A, Surfonic TDA-9®, and an oil having a Specific Gravity below 0.9

Another embodiment comprises cyclosporin A, Surfonic TX-CCN®, and an oil having a Specific Gravity below 0.9

Another embodiment comprises cyclosporin A, Tagat CH 40®, and an oil having a Specific Gravity below 0.9

Another embodiment comprises cyclosporin A, Tagat CH 60®, and an oil having a Specific Gravity below 0.9

Another embodiment comprises cyclosporin A, Tagat L 2®, and an oil having a Specific Gravity below 0.9

Another embodiment comprises cyclosporin A, Tagat S®, and an oil having a Specific Gravity below 0.9

Another embodiment comprises cyclosporin A, Tagat S 2®, and an oil having a Specific Gravity below 0.9

Another embodiment comprises cyclosporin A, Tally 100 Plus®, and an oil having a Specific Gravity below 0.9

Another embodiment comprises cyclosporin A, T-Det A106®, and an oil having a Specific Gravity below 0.9

Another embodiment comprises cyclosporin A, T-Det A109®, and an oil having a Specific Gravity below 0.9

Another embodiment comprises cyclosporin A, T-Det A136®, and an oil having a Specific Gravity below 0.9

Another embodiment comprises cyclosporin A, T-Det A139®, and an oil having a Specific Gravity below 0.9

Another embodiment comprises cyclosporin A, T-Det A2412®, and an oil having a Specific Gravity below 0.9

Another embodiment comprises cyclosporin A, T-Det A243®, and an oil having a Specific Gravity below 0.9

Another embodiment comprises cyclosporin A, T-Det A247®, and an oil having a Specific Gravity below 0.9

Another embodiment comprises cyclosporin A, T-Det A249®, and an oil having a Specific Gravity below 0.9

Another embodiment comprises cyclosporin A, T-Det A467®, and an oil having a Specific Gravity below 0.9

Another embodiment comprises cyclosporin A, T-Det COE®, and an oil having a Specific Gravity below 0.9

Another embodiment comprises cyclosporin A, T-Det DD-10®, and an oil having a Specific Gravity below 0.9

Another embodiment comprises cyclosporin A, T-Det DD-5®, and an oil having a Specific Gravity below 0.9

Another embodiment comprises cyclosporin A, T-Det DD-7®, and an oil having a Specific Gravity below 0.9

Another embodiment comprises cyclosporin A, T-Det EPO-104®, and an oil having a Specific Gravity below 0.9

Another embodiment comprises cyclosporin A, T-Det EPO-61®, and an oil having a Specific Gravity below 0.9

Another embodiment comprises cyclosporin A, T-Det EPO-62®, and an oil having a Specific Gravity below 0.9

Another embodiment comprises cyclosporin A, T-Det EPO-64®, and an oil having a Specific Gravity below 0.9

Another embodiment comprises cyclosporin A, T-Det N-1.5®, and an oil having a Specific Gravity below 0.9

Another embodiment comprises cyclosporin A, T-Det N-10.5®, and an oil having a Specific Gravity below 0.9

Another embodiment comprises cyclosporin A, T-Det N-100®, and an oil having a Specific Gravity below 0.9

Another embodiment comprises cyclosporin A, T-Det N-1007®, and an oil having a Specific Gravity below 0.9

Another embodiment comprises cyclosporin A, T-Det N-12®, and an oil having a Specific Gravity below 0.9

Another embodiment comprises cyclosporin A, T-Det N-14®, and an oil having a Specific Gravity below 0.9

Another embodiment comprises cyclosporin A, T-Det N-20®, and an oil having a Specific Gravity below 0.9

Another embodiment comprises cyclosporin A, T-Det N-30®, and an oil having a Specific Gravity below 0.9

Another embodiment comprises cyclosporin A, T-Det N-307®, and an oil having a Specific Gravity below 0.9

Another embodiment comprises cyclosporin A, T-Det N-4®, and an oil having a Specific Gravity below 0.9

Another embodiment comprises cyclosporin A, T-Det N-40®, and an oil having a Specific Gravity below 0.9

Another embodiment comprises cyclosporin A, T-Det N-407®, and an oil having a Specific Gravity below 0.9

Another embodiment comprises cyclosporin A, T-Det N-50®, and an oil having a Specific Gravity below 0.9

Another embodiment comprises cyclosporin A, T-Det N-507®, and an oil having a Specific Gravity below 0.9

Another embodiment comprises cyclosporin A, T-Det N-6®, and an oil having a Specific Gravity below 0.9

Another embodiment comprises cyclosporin A, T-Det N-70®, and an oil having a Specific Gravity below 0.9

Another embodiment comprises cyclosporin A, T-Det N-8®, and an oil having a Specific Gravity below 0.9

Another embodiment comprises cyclosporin A, T-Det N-9.5®, and an oil having a Specific Gravity below 0.9

Another embodiment comprises cyclosporin A, T-Det O-12®, and an oil having a Specific Gravity below 0.9

Another embodiment comprises cyclosporin A, T-Det O-165®, and an oil having a Specific Gravity below 0.9

Another embodiment comprises cyclosporin A, T-Det O-307®, and an oil having a Specific Gravity below 0.9

Another embodiment comprises cyclosporin A, T-Det O-40®, and an oil having a Specific Gravity below 0.9

Another embodiment comprises cyclosporin A, T-Det O-407®, and an oil having a Specific Gravity below 0.9

Another embodiment comprises cyclosporin A, T-Det O-6®, and an oil having a Specific Gravity below 0.9

Another embodiment comprises cyclosporin A, T-Det O-8®, and an oil having a Specific Gravity below 0.9

Another embodiment comprises cyclosporin A, T-Det O-9®, and an oil having a Specific Gravity below 0.9

Another embodiment comprises cyclosporin A, Tegin®, and an oil having a Specific Gravity below 0.9

Another embodiment comprises cyclosporin A, Tergitol 15-S-12®, and an oil having a Specific Gravity below 0.9

Another embodiment comprises cyclosporin A, Tergitol 15-S-15®, and an oil having a Specific Gravity below 0.9

Another embodiment comprises cyclosporin A, Tergitol 15-S-20®, and an oil having a Specific Gravity below 0.9

Another embodiment comprises cyclosporin A, Tergitol 15-S-20(80AQ)®, and an oil having a Specific Gravity below 0.9

Another embodiment comprises cyclosporin A, Tergitol 15-S-3®, and an oil having a Specific Gravity below 0.9

Another embodiment comprises cyclosporin A, Tergitol 15-S-30®, and an oil having a Specific Gravity below 0.9

Another embodiment comprises cyclosporin A, Tergitol 15-S-40®, and an oil having a Specific Gravity below 0.9

Another embodiment comprises cyclosporin A, Tergitol 15-S-40(70AQ)®, and an oil having a Specific Gravity below 0.9

Another embodiment comprises cyclosporin A, Tergitol 15-S-5®, and an oil having a Specific Gravity below 0.9

Another embodiment comprises cyclosporin A, Tergitol 15-S-7®, and an oil having a Specific Gravity below 0.9

Another embodiment comprises cyclosporin A, Tergitol 15S-9®, and an oil having a Specific Gravity below 0.9

Another embodiment comprises cyclosporin A, Tergitol L-101®, and an oil having a Specific Gravity below 0.9

Another embodiment comprises cyclosporin A, Tergitol L-61®, and an oil having a Specific Gravity below 0.9

Another embodiment comprises cyclosporin A, Tergitol L-62®, and an oil having a Specific Gravity below 0.9

Another embodiment comprises cyclosporin A, Tergitol L-64®, and an oil having a Specific Gravity below 0.9

Another embodiment comprises cyclosporin A, Tergitol L-81®, and an oil having a Specific Gravity below 0.9

Another embodiment comprises cyclosporin A, Tergitol NP-10®, and an oil having a Specific Gravity below 0.9

Another embodiment comprises cyclosporin A, Tergitol NP-11®, and an oil having a Specific Gravity below 0.9

Another embodiment comprises cyclosporin A, Tergitol NP-12®, and an oil having a Specific Gravity below 0.9

Another embodiment comprises cyclosporin A, Tergitol NP-13®, and an oil having a Specific Gravity below 0.9

Another embodiment comprises cyclosporin A, Tergitol NP-15®, and an oil having a Specific Gravity below 0.9

Another embodiment comprises cyclosporin A, Tergitol NP-30®, and an oil having a Specific Gravity below 0.9

Another embodiment comprises cyclosporin A, Tergitol NP-30 (70 AQ)®, and an oil having a Specific Gravity below 0.9

Another embodiment comprises cyclosporin A, Tergitol NP-4®, and an oil having a Specific Gravity below 0.9

Another embodiment comprises cyclosporin A, Tergitol NP-40®, and an oil having a Specific Gravity below 0.9

Another embodiment comprises cyclosporin A, Tergitol NP-40 (70 AQ)®, and an oil having a Specific Gravity below 0.9

Another embodiment comprises cyclosporin A, Tergitol NP-50 (70 AQ)®, and an oil having a Specific Gravity below 0.9

Another embodiment comprises cyclosporin A, Tergitol NP-55 (70 AQ)®, and an oil having a Specific Gravity below 0.9

Another embodiment comprises cyclosporin A, Tergitol NP-6®, and an oil having a Specific Gravity below 0.9

Another embodiment comprises cyclosporin A, Tergitol NP-7®, and an oil having a Specific Gravity below 0.9

Another embodiment comprises cyclosporin A, Tergitol NP-70 (70 AQ)®, and an oil having a Specific Gravity below 0.9

Another embodiment comprises cyclosporin A, Tergitol NP-8®, and an oil having a Specific Gravity below 0.9

Another embodiment comprises cyclosporin A, Tergitol NP-9®, and an oil having a Specific Gravity below 0.9

Another embodiment comprises cyclosporin A, Tergitol NP-9.5®, and an oil having a Specific Gravity below 0.9

Another embodiment comprises cyclosporin A, Tergitol TMN-10®, and an oil having a Specific Gravity below 0.9

Another embodiment comprises cyclosporin A, Tergitol TMN-3®, and an oil having a Specific Gravity below 0.9

Another embodiment comprises cyclosporin A, Tergitol TMN-6®, and an oil having a Specific Gravity below 0.9

Another embodiment comprises cyclosporin A, Tergitol XD®, and an oil having a Specific Gravity below 0.9

Another embodiment comprises cyclosporin A, Tergitol XH®, and an oil having a Specific Gravity below 0.9

Another embodiment comprises cyclosporin A, Tergitol XJ®, and an oil having a Specific Gravity below 0.9

Another embodiment comprises cyclosporin A, Tetronic 1107®, and an oil having a Specific Gravity below 0.9

Another embodiment comprises cyclosporin A, Tetronic 1307®, and an oil having a Specific Gravity below 0.9

Another embodiment comprises cyclosporin A, Tetronic 150R1®, and an oil having a Specific Gravity below 0.9

Another embodiment comprises cyclosporin A, Tetronic 304®, and an oil having a Specific Gravity below 0.9

Another embodiment comprises cyclosporin A, Tetronic 701®, and an oil having a Specific Gravity below 0.9

Another embodiment comprises cyclosporin A, Tetronic 901®, and an oil having a Specific Gravity below 0.9

Another embodiment comprises cyclosporin A, Tetronic 904®, and an oil having a Specific Gravity below 0.9

Another embodiment comprises cyclosporin A, Tetronic 908®, and an oil having a Specific Gravity below 0.9

Another embodiment comprises cyclosporin A, Tetronic 90R43®, and an oil having a Specific Gravity below 0.9

Another embodiment comprises cyclosporin A, Tomadol 1-3®, and an oil having a Specific Gravity below 0.9

Another embodiment comprises cyclosporin A, Tomadol 1-5®, and an oil having a Specific Gravity below 0.9

Another embodiment comprises cyclosporin A, Tomadol 1-7®, and an oil having a Specific Gravity below 0.9

Another embodiment comprises cyclosporin A, Tomadol 23-1®, and an oil having a Specific Gravity below 0.9

Another embodiment comprises cyclosporin A, Tomadol 23-3®, and an oil having a Specific Gravity below 0.9

Another embodiment comprises cyclosporin A, Tomadol 23-5®, and an oil having a Specific Gravity below 0.9

Another embodiment comprises cyclosporin A, Tomadol 23-6.5®, and an oil having a Specific Gravity below 0.9

Another embodiment comprises cyclosporin A, Tomadol 25-12®, and an oil having a Specific Gravity below 0.9

Another embodiment comprises cyclosporin A, Tomadol 25-3®, and an oil having a Specific Gravity below 0.9

Another embodiment comprises cyclosporin A, Tomadol 25-7®, and an oil having a Specific Gravity below 0.9

Another embodiment comprises cyclosporin A, Tomadol 25-9®, and an oil having a Specific Gravity below 0.9

Another embodiment comprises cyclosporin A, Tomadol 45-13®, and an oil having a Specific Gravity below 0.9

Another embodiment comprises cyclosporin A, Tomadol 45-2.5®, and an oil having a Specific Gravity below 0.9

Another embodiment comprises cyclosporin A, Tomadol 45-7®, and an oil having a Specific Gravity below 0.9

Another embodiment comprises cyclosporin A, Tomadol 91-2.5®, and an oil having a Specific Gravity below 0.9

Another embodiment comprises cyclosporin A, Tomadol 91-6®, and an oil having a Specific Gravity below 0.9

Another embodiment comprises cyclosporin A, Tomadol 91 -8®, and an oil having a Specific Gravity below 0.9

Another embodiment comprises cyclosporin A, Triton X-100®, and an oil having a Specific Gravity below 0.9

Another embodiment comprises cyclosporin A, Triton X-102®, and an oil having a Specific Gravity below 0.9

Another embodiment comprises cyclosporin A, Triton X-114®, and an oil having a Specific Gravity below 0.9

Another embodiment comprises cyclosporin A, Triton X-15®, and an oil having a Specific Gravity below 0.9

Another embodiment comprises cyclosporin A, Triton X-165 (70%)®, and an oil having a Specific Gravity below 0.9

Another embodiment comprises cyclosporin A, Triton X-207®, and an oil having a Specific Gravity below 0.9

Another embodiment comprises cyclosporin A, Triton X-305 (70%)®, and an oil having a Specific Gravity below 0.9

Another embodiment comprises cyclosporin A, Triton X-35®, and an oil having a Specific Gravity below 0.9

Another embodiment comprises cyclosporin A, Triton X-405 (70%)®, and an oil having a Specific Gravity below 0.9

Another embodiment comprises cyclosporin A, Triton X-45®, and an oil having a Specific Gravity below 0.9

Another embodiment comprises cyclosporin A, Triton X-705 (70%)®, and an oil having a Specific Gravity below 0.9

Another embodiment comprises cyclosporin A, Volpo S-10®, and an oil having a Specific Gravity below 0.9

Another embodiment comprises cyclosporin A, Volpo S-2®, and an oil having a Specific Gravity below 0.9

Another embodiment comprises cyclosporin A, Volpo S-20®, and an oil having a Specific Gravity below 0.9

Another embodiment comprises cyclosporin A, Volpo-10®, and an oil having a Specific Gravity below 0.9

Another embodiment comprises cyclosporin A, Volpo-20®, and an oil having a Specific Gravity below 0.9

Another embodiment comprises cyclosporin A, Volpo-3®, and an oil having a Specific Gravity below 0.9

Another embodiment comprises cyclosporin A, Volpo-5®, and an oil having a Specific Gravity below 0.9

Another embodiment comprises cyclosporin A, Abex 2515®, and Mineral oil.

Another embodiment comprises cyclosporin A, Abex 2525/40®, and Mineral oil.

Another embodiment comprises cyclosporin A, Abex 2535®, and Mineral oil.

Another embodiment comprises cyclosporin A, Abex 2545®, and Mineral oil.

Another embodiment comprises cyclosporin A, Acconon E®, and Mineral oil.

Another embodiment comprises cyclosporin A, Alfonic 1216CO-9®, and Mineral oil.

Another embodiment comprises cyclosporin A, Alfonic 1412-3®, and Mineral oil.

Another embodiment comprises cyclosporin A, Alfonic TDA-12®, and Mineral oil.

Another embodiment comprises cyclosporin A, Alfonic TDA-3®, and Mineral oil.

Another embodiment comprises cyclosporin A, Alfonic TDA-4®, and Mineral oil.

Another embodiment comprises cyclosporin A, Alfonic TDA-6®, and Mineral oil.

Another embodiment comprises cyclosporin A, Alfonic TDA-7®, and Mineral oil.

Another embodiment comprises cyclosporin A, Alfonic TDA-8®, and Mineral oil.

Another embodiment comprises cyclosporin A, Alfonic TDA-9®, and Mineral oil.

Another embodiment comprises cyclosporin A, Alkamuls 400-DO®, and Mineral oil.

Another embodiment comprises cyclosporin A, Alkamuls 400-MO/E®, and Mineral oil.

Another embodiment comprises cyclosporin A, Ameroxol OE-10®, and Mineral oil.

Another embodiment comprises cyclosporin A, Ameroxol OE-2®, and Mineral oil.

Another embodiment comprises cyclosporin A, Ameroxol OE-20®, and Mineral oil.

Another embodiment comprises cyclosporin A, Armix 176®, and Mineral oil.

Another embodiment comprises cyclosporin A, Armix 180-C®, and Mineral oil.

Another embodiment comprises cyclosporin A, Armix 183®, and Mineral oil.

Another embodiment comprises cyclosporin A, Brij 35®, and Mineral oil.

Another embodiment comprises cyclosporin A, Brij 35 Liquid®, and Mineral oil.

Another embodiment comprises cyclosporin A, Brij 35 SP®, and Mineral oil.

Another embodiment comprises cyclosporin A, Canasol MJ 2109®, and Mineral oil.

Another embodiment comprises cyclosporin A, Canasol MJ 52®, and Mineral oil.

Another embodiment comprises cyclosporin A, Canasol R 3600®, and Mineral oil.

Another embodiment comprises cyclosporin A, Canasol R 3603®, and Mineral oil.

Another embodiment comprises cyclosporin A, Canasol R 4000 H®, and Mineral oil.

Another embodiment comprises cyclosporin A, Cedepal CA-210®, and Mineral oil.

Another embodiment comprises cyclosporin A, Cedepal CA-520®, and Mineral oil.

Another embodiment comprises cyclosporin A, Cedepal CA-630®, and Mineral oil.

Another embodiment comprises cyclosporin A, Cedepal CA-720®, and Mineral oil.

Another embodiment comprises cyclosporin A, Cedepal CA-890®, and Mineral oil.

Another embodiment comprises cyclosporin A, Cedepal CA-897®, and Mineral oil.

Another embodiment comprises cyclosporin A, Cedepal CO-210®, and Mineral oil.

Another embodiment comprises cyclosporin A, Cedepal CO-430®, and Mineral oil.

Another embodiment comprises cyclosporin A, Cedepal CO-530®, and Mineral oil.

Another embodiment comprises cyclosporin A, Cedepal CO-630®, and Mineral oil.

Another embodiment comprises cyclosporin A, Cedepal CO-710®, and Mineral oil.

Another embodiment comprises cyclosporin A, Cedepal CO-730®, and Mineral oil.

Another embodiment comprises cyclosporin A, Cedepal CO-880®, and Mineral oil.

Another embodiment comprises cyclosporin A, Cedepal CO-887®, and Mineral oil.

Another embodiment comprises cyclosporin A, Cedepal CO-890®, and Mineral oil.

Another embodiment comprises cyclosporin A, Cedepal CO-897®, and Mineral oil.

Another embodiment comprises cyclosporin A, Cedepal CO-970®, and Mineral oil.

Another embodiment comprises cyclosporin A, Cedepal CO-977®, and Mineral oil.

Another embodiment comprises cyclosporin A, Cedepal CO-990®, and Mineral oil.

Another embodiment comprises cyclosporin A, Cedepal CO-997®, and Mineral oil.

Another embodiment comprises cyclosporin A, Chemal BP 261®, and Mineral oil.

Another embodiment comprises cyclosporin A, Chemal BP 262®, and Mineral oil.

Another embodiment comprises cyclosporin A, Chemal BP 3172®, and Mineral oil.

Another embodiment comprises cyclosporin A, Chemal BP 3174®, and Mineral oil.

Another embodiment comprises cyclosporin A, Chemal DA-4®, and Mineral oil.

Another embodiment comprises cyclosporin A, Chemal DA-6®, and Mineral oil.

Another embodiment comprises cyclosporin A, Chemal DA-9®, and Mineral oil.

Another embodiment comprises cyclosporin A, Chemal LA-23®, and Mineral oil.

Another embodiment comprises cyclosporin A, Chemal LA-4®, and Mineral oil.

Another embodiment comprises cyclosporin A, Chemal LA-9®, and Mineral oil.

Another embodiment comprises cyclosporin A, Chemal OA-20G®, and Mineral oil.

Another embodiment comprises cyclosporin A, Chemal OA-5®, and Mineral oil.

Another embodiment comprises cyclosporin A, Chemal OA-9®, and Mineral oil.

Another embodiment comprises cyclosporin A, Chemal TDA-12®, and Mineral oil.

Another embodiment comprises cyclosporin A, Chemal TDA-15®, and Mineral oil.

Another embodiment comprises cyclosporin A, Chemal TDA-3®, and Mineral oil.

Another embodiment comprises cyclosporin A, Chemal TDA-6®, and Mineral oil.

Another embodiment comprises cyclosporin A, Chemal TDA-9®, and Mineral oil.

Another embodiment comprises cyclosporin A, Chemax CO-16®, and Mineral oil.

Another embodiment comprises cyclosporin A, Chemax CO-200/50®, and Mineral oil.

Another embodiment comprises cyclosporin A, Chemax CO-25®, and Mineral oil.

Another embodiment comprises cyclosporin A, Chemax CO-30®, and Mineral oil.

Another embodiment comprises cyclosporin A, Chemax CO-36®, and Mineral oil.

Another embodiment comprises cyclosporin A, Chemax CO-40®, and Mineral oil.

Another embodiment comprises cyclosporin A, Chemax CO-5®, and Mineral oil.

Another embodiment comprises cyclosporin A, Chemax CO-80®, and Mineral oil.

Another embodiment comprises cyclosporin A, Chemax DNP-150/50®, and Mineral oil.

Another embodiment comprises cyclosporin A, Chemax DNP-18®, and Mineral oil.

Another embodiment comprises cyclosporin A, Chemax DNP-8®, and Mineral oil.

Another embodiment comprises cyclosporin A, Chemax E 200 ML®, and Mineral oil.

Another embodiment comprises cyclosporin A, Chemax E 200 MO®, and Mineral oil.

Another embodiment comprises cyclosporin A, Chemax E 400 ML®, and Mineral oil.

Another embodiment comprises cyclosporin A, Chemax E 400 MO®,200 , and Mineral oil.

Another embodiment comprises cyclosporin A, Chemax E 600 ML®, and Mineral oil.

Another embodiment comprises cyclosporin A, Chemax E 600 MO®, and Mineral oil.

Another embodiment comprises cyclosporin A, Chemax E 1000 MO®, and Mineral oil.

Another embodiment comprises cyclosporin A, Chemax E 1000 MS®, and Mineral oil.

Another embodiment comprises cyclosporin A, Chemax E 200 MS®, and Mineral oil.

Another embodiment comprises cyclosporin A, Chemax E 400 MS®, and Mineral oil.

Another embodiment comprises cyclosporin A, Chemax E 600 MS®, and Mineral oil.

Another embodiment comprises cyclosporin A, Chemax HCO-16®, and Mineral oil.

Another embodiment comprises cyclosporin A, Chemax HCO-200/50®, and Mineral oil.

Another embodiment comprises cyclosporin A, Chemax HCO-25®, and Mineral oil.

Another embodiment comprises cyclosporin A, Chemax HCO-5®, and Mineral oil.

Another embodiment comprises cyclosporin A, Chemax TO-16®, and Mineral oil.

Another embodiment comprises cyclosporin A, Chemax TO-8®, and Mineral oil.

Another embodiment comprises cyclosporin A, Colalube 3409®, and Mineral oil.

Another embodiment comprises cyclosporin A, Colalube 3411®, and Mineral oil.

Another embodiment comprises cyclosporin A, Colalube 3413®, and Mineral oil.

Another embodiment comprises cyclosporin A, Colalube 3414®, and Mineral oil.

Another embodiment comprises cyclosporin A, Colalube 3415®, and Mineral oil.

Another embodiment comprises cyclosporin A, Colalube 3416®, and Mineral oil.

Another embodiment comprises cyclosporin A, Colalube 3417®, and Mineral oil.

Another embodiment comprises cyclosporin A, Delonic NPE-100®, and Mineral oil.

Another embodiment comprises cyclosporin A, Delonic NPE-30®, and Mineral oil.

Another embodiment comprises cyclosporin A, Delonic NPE-40®, and Mineral oil.

Another embodiment comprises cyclosporin A, Delonic OPE-10®, and Mineral oil.

Another embodiment comprises cyclosporin A, Delonic OPE-12®, and Mineral oil.

Another embodiment comprises cyclosporin A, Delonic OPE-30®, and Mineral oil.

Another embodiment comprises cyclosporin A, Delonic OPE-40®, and Mineral oil.

Another embodiment comprises cyclosporin A, Delonic OPE-5®, and Mineral oil.

Another embodiment comprises cyclosporin A, Delonic OPE-7.5®, and Mineral oil.

Another embodiment comprises cyclosporin A, DePeg 16-CO®, and Mineral oil.

Another embodiment comprises cyclosporin A, DePeg 25-C®, and Mineral oil.

Another embodiment comprises cyclosporin A, DePeg 30-CO®, and Mineral oil.

Another embodiment comprises cyclosporin A, DePeg 40-CO®, and Mineral oil.

Another embodiment comprises cyclosporin A, DePeg 5-CO®, and Mineral oil.

Another embodiment comprises cyclosporin A, DePeg 80-CO®, and Mineral oil.

Another embodiment comprises cyclosporin A, DeSonic S-100®, and Mineral oil.

Another embodiment comprises cyclosporin A, DeSonic S-114®, and Mineral oil.

Another embodiment comprises cyclosporin A, DeSonic S-405®, and Mineral oil.

Another embodiment comprises cyclosporin A, DeSonic S-45®, and Mineral oil.

Another embodiment comprises cyclosporin A, DeSonic TDA-9®, and Mineral oil.

Another embodiment comprises cyclosporin A, DeThox Acid O-14®, and Mineral oil.

Another embodiment comprises cyclosporin A, DeThox Acid O-16®, and Mineral oil.

Another embodiment comprises cyclosporin A, DeThox Acid O-9®, and Mineral oil.

Another embodiment comprises cyclosporin A, DeThox Acid TO-14®, and Mineral oil.

Another embodiment comprises cyclosporin A, DeThox Acid TO-16.5®, and Mineral oil.

Another embodiment comprises cyclosporin A, DeThox Acid TO-8.5®, and Mineral oil.

Another embodiment comprises cyclosporin A, Dur-Em 114®, and Mineral oil.

Another embodiment comprises cyclosporin A, Dur-Em 117®, and Mineral oil.

Another embodiment comprises cyclosporin A, Dur-Em 204®, and Mineral oil.

Another embodiment comprises cyclosporin A, Dur-Em 207®, and Mineral oil.

Another embodiment comprises cyclosporin A, Dur-Em 207E®, and Mineral oil.

Another embodiment comprises cyclosporin A, Emerest 2704®, and Mineral oil.

Another embodiment comprises cyclosporin A, Emerest 2712®, and Mineral oil.

Another embodiment comprises cyclosporin A, Emerest 2715®, and Mineral oil.

Another embodiment comprises cyclosporin A, Ethal EH-2®, and Mineral oil.

Another embodiment comprises cyclosporin A, Ethal EH-5®, and Mineral oil.

Another embodiment comprises cyclosporin A, Ethal OA-23®, and Mineral oil.

Another embodiment comprises cyclosporin A, Ethosperse CA-20®, and Mineral oil.

Another embodiment comprises cyclosporin A, Ethosperse LA-12®, and Mineral oil.

Another embodiment comprises cyclosporin A, Ethosperse LA-23®, and Mineral oil.

Another embodiment comprises cyclosporin A, Ethosperse LA-4®, and Mineral oil.

Another embodiment comprises cyclosporin A, Ethox 3095®, and Mineral oil.

Another embodiment comprises cyclosporin A, Ethox CO-16®, and Mineral oil.

Another embodiment comprises cyclosporin A, Ethox CO-200®, and Mineral oil.

Another embodiment comprises cyclosporin A, Ethox CO-25®, and Mineral oil.

Another embodiment comprises cyclosporin A, Ethox CO-30®, and Mineral oil.

Another embodiment comprises cyclosporin A, Ethox CO-36®, and Mineral oil.

Another embodiment comprises cyclosporin A, Ethox CO-40®, and Mineral oil.

Another embodiment comprises cyclosporin A, Ethox CO-5®, and Mineral oil.

Another embodiment comprises cyclosporin A, Ethox DO-14®, and Mineral oil.

Another embodiment comprises cyclosporin A, Ethox DO-9®, and Mineral oil.

Another embodiment comprises cyclosporin A, Ethox MA-8®, and Mineral oil.

Another embodiment comprises cyclosporin A, Ethox MI-14®, and Mineral oil.

Another embodiment comprises cyclosporin A, Ethox ML-14®, and Mineral oil.

Another embodiment comprises cyclosporin A, Ethox ML-5®, and Mineral oil.

Another embodiment comprises cyclosporin A, Ethox MO-14®, and Mineral oil.

Another embodiment comprises cyclosporin A, Ethox MO-9®, and Mineral oil.

Another embodiment comprises cyclosporin A, Ethox MS-14®, and Mineral oil.

Another embodiment comprises cyclosporin A, Ethox MS-23®, and Mineral oil.

Another embodiment comprises cyclosporin A, Ethox MS-40®, and Mineral oil.

Another embodiment comprises cyclosporin A, Ethox MS-8®, and Mineral oil.

Another embodiment comprises cyclosporin A, Ethox TO-16®, and Mineral oil.

Another embodiment comprises cyclosporin A, Ethox TO-8®, and Mineral oil.

Another embodiment comprises cyclosporin A, Ethylan SN-120®, and Mineral oil.

Another embodiment comprises cyclosporin A, Ethylan SN-70®, and Mineral oil.

Another embodiment comprises cyclosporin A, Ethylan SN-90®, and Mineral oil.

Another embodiment comprises cyclosporin A, Flexricin 13®, and Mineral oil.

Another embodiment comprises cyclosporin A, Flexricin 15®, and Mineral oil.

Another embodiment comprises cyclosporin A, Genapol 1261®, and Mineral oil.

Another embodiment comprises cyclosporin A, Generol 122 E25®, and Mineral oil.

Another embodiment comprises cyclosporin A, Glicepol 160®, and Mineral oil.

Another embodiment comprises cyclosporin A, Glicepol 560®, and Mineral oil.

Another embodiment comprises cyclosporin A, Glicepol GMS 20®, and Mineral oil.

Another embodiment comprises cyclosporin A, Hetoxide BN-13®, and Mineral oil.

Another embodiment comprises cyclosporin A, Hetoxide DNP-4®, and Mineral oil.

Another embodjment comprises cyclosporin A, Hetoxide DNP-9.6®, and Mineral oil.

Another embodiment comprises cyclosporin A, Hetoxide G-26®, and Mineral oil.

Another embodiment comprises cyclosporin A, Hetoxide G-7®, and Mineral oil.

Another embodiment comprises cyclosporin A, Hetoxide GT-20®, and Mineral oil.

Another embodiment comprises cyclosporin A, Hetoxide HC-40®, and Mineral oil.

Another embodiment comprises cyclosporin A, Hetoxol C-24®, and Mineral oil.

Another embodiment comprises cyclosporin A, Hetoxol CA-10®, and Mineral oil.

Another embodiment comprises cyclosporin A, Hetoxol CA-2®, and Mineral oil.

Another embodiment comprises cyclosporin A, Hetoxol CA-20®, and Mineral oil.

Another embodiment comprises cyclosporin A, Hetoxol CAWS®, and Mineral oil.

Another embodiment comprises cyclosporin A, Hetoxol CD-3®, and Mineral oil.

Another embodiment comprises cyclosporin A, Hetoxol CD-4®, and Mineral oil.

Another embodiment comprises cyclosporin A, Hetoxol CS-15®, and Mineral oil.

Another embodiment comprises cyclosporin A, Hetoxol CS-20®, and Mineral oil.

Another embodiment comprises cyclosporin A, Hetoxol CS-20D®, and Mineral oil.

Another embodiment comprises cyclosporin A, Hetoxol CS-25®, and Mineral oil.

Another embodiment comprises cyclosporin A, Hetoxol CS-50®, and Mineral oil.

Another embodiment comprises cyclosporin A, Hetoxol CS-9®, and Mineral oil.

Another embodiment comprises cyclosporin A, Hetoxol CSA-15®, and Mineral oil.

Another embodiment comprises cyclosporin A, Hetoxol D®, and Mineral oil.

Another embodiment comprises cyclosporin A, Hetoxol G®, and Mineral oil.

Another embodiment comprises cyclosporin A, Hetoxol J®, and Mineral oil.

Another embodiment comprises cyclosporin A, Hetoxol L®and Mineral oil.

Another embodiment comprises cyclosporin A, Hetoxol L-23®, and Mineral oil.

Another embodiment comprises cyclosporin A, Hetoxol L-4®, and Mineral oil.

Another embodiment comprises cyclosporin A, Hetoxol L-9®, and Mineral oil.

Another embodiment comprises cyclosporin A, Hetoxol LS-9®, and Mineral oil.

Another embodiment comprises cyclosporin A, Hetoxol M-3®, and Mineral oil.

Another embodiment comprises cyclosporin A, Hetoxol OA-10 Special®, and Mineral oil.

Another embodiment comprises cyclosporin A, Hetoxol OA-20 Special®, and Mineral oil.

Another embodiment comprises cyclosporin A, Hetoxol OA-3 Special®, and Mineral oil.

Another embodiment comprises cyclosporin A, Hetoxol OA-5 Special®, and Mineral oil.

Another embodiment comprises cyclosporin A, Hetoxol OL-10®, and Mineral oil.

Another embodiment comprises cyclosporin A, Hetoxol OL-2®, and Mineral oil.

Another embodiment comprises cyclosporin A, Hetoxol OL-23®, and Mineral oil.

Another embodiment comprises cyclosporin A, Hetoxol OL-4®, and Mineral oil.

Another embodiment comprises cyclosporin A, Hetoxol OL-40®, and Mineral oil.

Another embodiment comprises cyclosporin A, Hetoxol OL-5®, and Mineral oil.

Another embodiment comprises cyclosporin A, Hetoxol PLA®, and Mineral oil.

Another embodiment comprises cyclosporin A, Hetoxol SP-15®, and Mineral oil.

Another embodiment comprises cyclosporin A, Hetoxol STA-10®, and Mineral oil.

Another embodiment comprises cyclosporin A, Hetoxol STA-2®, and Mineral oil.

Another embodiment comprises cyclosporin A, Hetoxol STA-30®, and Mineral oil.

Another embodiment comprises cyclosporin A, Hetoxol TD-12®, and Mineral oil.

Another embodiment comprises cyclosporin A, Hetoxol TD-18®, and Mineral oil.

Another embodiment comprises cyclosporin A, Hetoxol TD-3®, and Mineral oil.

Another embodiment comprises cyclosporin A, Hetoxol TD-6®, and Mineral oil.

Another embodiment comprises cyclosporin A, Hetoxol TD-9®, and Mineral oil.

Another embodiment comprises cyclosporin A, Hetoxol TDEP-15®, and Mineral oil.

Another embodiment comprises cyclosporin A, Hetoxol TDEP-63®, and Mineral oil.

Another embodiment comprises cyclosporin A, Iconol 24-12®, and Mineral oil.

Another embodiment comprises cyclosporin A, Iconol 24-23®, and Mineral oil.

Another embodiment comprises cyclosporin A, Iconol 24-4®, and Mineral oil.

Another embodiment comprises cyclosporin A, Iconol DA-4®, and Mineral oil.

Another embodiment comprises cyclosporin A, Iconol DA-6®, and Mineral oil.

Another embodiment comprises cyclosporin A, Iconol TDA-10®, and Mineral oil.

Another embodiment comprises cyclosporin A, Iconol TDA-3®, and Mineral oil.

Another embodiment comprises cyclosporin A, Iconol TDA-6®, and Mineral oil.

Another embodiment comprises cyclosporin A, Iconol TDA-8-90%®, and Mineral oil.

Another embodiment comprises cyclosporin A, Iconol TDA-9®, and Mineral oil.

Another embodiment comprises cyclosporin A, Igepal CA-210®, and Mineral oil.

Another embodiment comprises cyclosporin A, Igepal CA-407®, and Mineral oil.

Another embodiment comprises cyclosporin A, Igepal CA-420®, and Mineral oil.

Another embodiment comprises cyclosporin A, Igepal CA-520®, and Mineral oil.

Another embodiment comprises cyclosporin A, Igepal CA-620®, and Mineral oil.

Another embodiment comprises cyclosporin A, Igepal CA-630®, and Mineral oil.

Another embodiment comprises cyclosporin A, Igepal CA-720®, and Mineral oil.

Another embodiment comprises cyclosporin A, Igepal CA-877®, and Mineral oil.

Another embodiment comprises cyclosporin A, Igepal CA-887®, and Mineral oil.

Another embodiment comprises cyclosporin A, Igepal CA-897®, and Mineral oil.

Another embodiment comprises cyclosporin A, Igepal CO-210®, and Mineral oil.

Another embodiment comprises cyclosporin A, Igepal CO-430®, and Mineral oil.

Another embodiment comprises cyclosporin A, Igepal CO-520®, and Mineral oil.

Another embodiment comprises cyclosporin A, Igepal CO-530®, and Mineral oil.

Another embodiment comprises cyclosporin A, Igepal CO-610®, and Mineral oil.

Another embodiment comprises cyclosporin A, Igepal CO-630®, and Mineral oil.

Another embodiment comprises cyclosporin A, Igepal CO-630 Special®, and Mineral oil.

Another embodiment comprises cyclosporin A, Igepal CO-660®, and Mineral oil.

Another embodiment comprises cyclosporin A, Igepal CO-710®, and Mineral oil.

Another embodiment comprises cyclosporin A, Igepal CO-720®, and Mineral oil.

Another embodiment comprises cyclosporin A, Igepal CO-730®, and Mineral oil.

Another embodiment comprises cyclosporin A, Igepal CO-738®, and Mineral oil.

Another embodiment comprises cyclosporin A, Igepal CO-850®, and Mineral oil.

Another embodiment comprises cyclosporin A, Igepal CO-880®, and Mineral oil.

Another embodiment comprises cyclosporin A, Igepal CO-887®, and Mineral oil.

Another embodiment comprises cyclosporin A, Igepal CO-890®, and Mineral oil.

Another embodiment comprises cyclosporin A, Igepal CO-897®, and Mineral oil.

Another embodiment comprises cyclosporin A, Igepal CO-970®, and Mineral oil.

Another embodiment comprises cyclosporin A, Igepal CO-977®, and Mineral oil.

Another embodiment comprises cyclosporin A, Igepal CO-987®, and Mineral oil.

Another embodiment comprises cyclosporin A, Igepal CO-990 FLK®, and Mineral oil.

Another embodiment comprises cyclosporin A, Igepal CO-997®, and Mineral oil.

Another embodiment comprises cyclosporin A, Igepal DM-430®, and Mineral oil.

Another embodiment comprises cyclosporin A, Igepal DM-530®, and Mineral oil.

Another embodiment comprises cyclosporin A, Igepal DM-710®, and Mineral oil.

Another embodiment comprises cyclosporin A, Igepal DM-730®, and Mineral oil.

Another embodiment comprises cyclosporin A, Igepal DM-880®, and Mineral oil.

Another embodiment comprises cyclosporin A, Igepal DM-970 FLK®, and Mineral oil.

Another embodiment comprises cyclosporin A, Igepal OD-410®, and Mineral oil.

Another embodiment comprises cyclosporin A, Igepal RC-520®, and Mineral oil.

Another embodiment comprises cyclosporin A, Igepal RC-620®, and Mineral oil.

Another embodiment comprises cyclosporin A, Igepal RC-630®, and Mineral oil.

Another embodiment comprises cyclosporin A, Lexemul CS-20®, and Mineral oil.

Another embodiment comprises cyclosporin A, Lipo GMS 450®, and Mineral oil.

Another embodiment comprises cyclosporin A, Lipo Polyglycol 1000®, and Mineral oil.

Another embodiment comprises cyclosporin A, Lipo Polyglycol 200®, and Mineral oil.

Another embodiment comprises cyclosporin A, Lipo Polyglycol 300®, and Mineral oil.

Another embodiment comprises cyclosporin A, Lipo Polyglycol 3350®, and Mineral oil.

Another embodiment comprises cyclosporin A, Lipo Polyglycol 400®, and Mineral oil.

Another embodiment comprises cyclosporin A, Lipo Polyglycol 600®, and Mineral oil.

Another embodiment comprises cyclosporin A, Lipocol C-10®, and Mineral oil.

Another embodiment comprises cyclosporin A, Lipocol C-2®, and Mineral oil.

Another embodiment comprises cyclosporin A, Lipocol C-20®, and Mineral oil.

Another embodiment comprises cyclosporin A, Lipocol L-12®, and Mineral oil.

Another embodiment comprises cyclosporin A, Lipocol L-23®, and Mineral oil.

Another embodiment comprises cyclosporin A, Lipocol L-4®, and Mineral oil.

Another embodiment comprises cyclosporin A, Lipocol O-10®, and Mineral oil.

Another embodiment comprises cyclosporin A, Lipocol O-2®, and Mineral oil.

Another embodiment comprises cyclosporin A, Lipocol O-20®, and Mineral oil.

Another embodiment comprises cyclosporin A, Lipocol O-5®, and Mineral oil.

Another embodiment comprises cyclosporin A, Lipocol S-10®, and Mineral oil.

Another embodiment comprises cyclosporin A, Lipocol S-2®, and Mineral oil.

Another embodiment comprises cyclosporin A, Lipocol S-20®, and Mineral oil.

Another embodiment comprises cyclosporin A, Lipocol SC-15®, and Mineral oil.

Another embodiment comprises cyclosporin A, Lipocol SC-20®, and Mineral oil.

Another embodiment comprises cyclosporin A, Lipocol SC-4®, and Mineral oil.

Another embodiment comprises cyclosporin A, Lipocol TD-12®, and Mineral oil.

Another embodiment comprises cyclosporin A, Lumulse 40-O K®, and Mineral oil.

Another embodiment comprises cyclosporin A, Lumulse 42-O K®, and Mineral oil.

Another embodiment comprises cyclosporin A, Lumulse GMT-40®, and Mineral oil.

Another embodiment comprises cyclosporin A, Lumulse PEG 1450®, and Mineral oil.

Another embodiment comprises cyclosporin A, Lumulse PEG 1450 NF®, and Mineral oil.

Another embodiment comprises cyclosporin A, Lumulse PEG 8000®, and Mineral oil.

Another embodiment comprises cyclosporin A, Lumulse PGO®, and Mineral oil.

Another embodiment comprises cyclosporin A, Lumulse POE (2) Oleyl Amine®, and Mineral oil.

Another embodiment comprises cyclosporin A, Magrabar PGO-104®, and Mineral oil.

Another embodiment comprises cyclosporin A, Mapeg 200 ML®, and Mineral oil.

Another embodiment comprises cyclosporin A, Mapeg 400 MO®, and Mineral oil.

Another embodiment comprises cyclosporin A, Mapeg 400 MOT®, and Mineral oil.

Another embodiment comprises cyclosporin A, Mapeg 400DO®, and Mineral oil.

Another embodiment comprises cyclosporin A, Mapeg 400DOT®, and Mineral oil.

Another embodiment comprises cyclosporin A, Mapeg 600DOT®, and Mineral oil.

Another embodiment comprises cyclosporin A, Marlox B 24/80®, and Mineral oil.

Another embodiment comprises cyclosporin A, Marlox FK 86®, and Mineral oil.

Another embodiment comprises cyclosporin A, Marlox MO 154®, and Mineral oil.

Another embodiment comprises cyclosporin A, Mazox CDA CG®, and Mineral oil.

Another embodiment comprises cyclosporin A, Monoglycerides Glyceryl Monestearate®, and Mineral oil.

Another embodiment comprises cyclosporin A, Myrj 45®, and Mineral oil.

Another embodiment comprises cyclosporin A, Myrj 52®, and Mineral oil.

Another embodiment comprises cyclosporin A, Myrj 52S®, and Mineral oil.

Another embodiment comprises cyclosporin A, Myrj 53®, and Mineral oil.

Another embodiment comprises cyclosporin A, Myrj 59®, and Mineral oil.

Another embodiment comprises cyclosporin A, Norfox NP-12®, and Mineral oil.

Another embodiment comprises cyclosporin A, Norfox NP-4®, and Mineral oil.

Another embodiment comprises cyclosporin A, Norfox NP-6®, and Mineral oil.

Another embodiment comprises cyclosporin A, Norfox NP-887®, and Mineral oil.

Another embodiment comprises cyclosporin A, Norfox NP-9®, and Mineral oil.

Another embodiment comprises cyclosporin A, Norfox NP-977®, and Mineral oil.

Another embodiment comprises cyclosporin A, Norfox OP-100®, and Mineral oil.

Another embodiment comprises cyclosporin A, Norfox OP-102®, and Mineral oil.

Another embodiment comprises cyclosporin A, Norfox OP-114®, and Mineral oil.

Another embodiment comprises cyclosporin A, Norfox OP-45®, and Mineral oil.

Another embodiment comprises cyclosporin A, Pegosperse 100L®, and Mineral oil.

Another embodiment comprises cyclosporin A, Pegosperse 100-O®, and Mineral oil.

Another embodiment comprises cyclosporin A, Pegosperse 100S®, and Mineral oil.

Another embodiment comprises cyclosporin A, Pegosperse 1500-MS®, and Mineral oil.

Another embodiment comprises cyclosporin A, Pegosperse 1750-MS®, and Mineral oil.

Another embodiment comprises cyclosporin A, Pegosperse 200 DL®, and Mineral oil.

Another embodiment comprises cyclosporin A, Pegosperse 200-ML®, and Mineral oil.

Another embodiment comprises cyclosporin A, Pegosperse 400-DL®, and Mineral oil.

Another embodiment comprises cyclosporin A, Pegosperse 400-DO®, and Mineral oil.

Another embodiment comprises cyclosporin A, Pegosperse 400-DS®, and Mineral oil.

Another embodiment comprises cyclosporin A, Pegosperse 400-ML®, and Mineral oil.

Another embodiment comprises cyclosporin A, Pegosperse 400-MO®, and Mineral oil.

Another embodiment comprises cyclosporin A, Pegosperse 400-MOT®, and Mineral oil.

Another embodiment comprises cyclosporin A, Pegosperse 400-MS®, and Mineral oil.

Another embodiment comprises cyclosporin A, Pegosperse 50-DS®, and Mineral oil.

Another embodiment comprises cyclosporin A, Pegosperse 50-MS®, and Mineral oil.

Another embodiment comprises cyclosporin A, Pegosperse 600-ML®, and Mineral oil.

Another embodiment comprises cyclosporin A, Pegosperse 600-MS®, and Mineral oil.

Another embodiment comprises cyclosporin A, Plurafac A-38®, and Mineral oil.

Another embodiment comprises cyclosporin A, Plurafac B-25-5®, and Mineral oil.

Another embodiment comprises cyclosporin A, Plurafac B-26®, and Mineral oil.

Another embodiment comprises cyclosporin A, Plurafac D-25®, and Mineral oil.

Another embodiment comprises cyclosporin A, Plurafac LF 131®, and Mineral oil.

Another embodiment comprises cyclosporin A, Plurafac LF 4030®, and Mineral oil.

Another embodiment comprises cyclosporin A, Plurafac LF 7000®, and Mineral oil.

Another embodiment comprises cyclosporin A, Plurafac RA-20®, and Mineral oil.

Another embodiment comprises cyclosporin A, Plurafac RA-30®, and Mineral oil.

Another embodiment comprises cyclosporin A, Plurafac RA-40®, and Mineral oil.

Another embodiment comprises cyclosporin A, Pluronic 10R5®, and Mineral oil.

Another embodiment comprises cyclosporin A, Pluronic 17R2®, and Mineral oil.

Another embodiment comprises cyclosporin A, Pluronic 17R49®, and Mineral oil.

Another embodiment comprises cyclosporin A, Pluronic 25R2®, and Mineral oil.

Another embodiment comprises cyclosporin A, Pluronic 25R4®, and Mineral oil.

Another embodiment comprises cyclosporin A, Pluronic 31R1®, and Mineral oil.

Another embodiment comprises cyclosporin A, Pluronic F108®, and Mineral oil.

Another embodiment comprises cyclosporin A, Pluronic F127®, and Mineral oil.

Another embodiment comprises cyclosporin A, Pluronic F38®, and Mineral oil.

Another embodiment comprises cyclosporin A, Pluronic F68®, and Mineral oil.

Another embodiment comprises cyclosporin A, Pluronic F68LF®, and Mineral oil.

Another embodiment comprises cyclosporin A, Pluronic F77®, and Mineral oil.

Another embodiment comprises cyclosporin A, Pluronic F87®, and Mineral oil.

Another embodiment comprises cyclosporin A, Pluronic F88®, and Mineral oil.

Another embodiment comprises cyclosporin A, Pluronic F98®, and Mineral oil.

Another embodiment comprises cyclosporin A, Pluronic L10®, and Mineral oil.

Another embodiment comprises cyclosporin A, Pluronic L101®, and Mineral oil.

Another embodiment comprises cyclosporin A, Pluronic L121®, and Mineral oil.

Another embodiment comprises cyclosporin A, Pluronic L31®, and Mineral oil.

Another embodiment comprises cyclosporin A, Pluronic L35®, and Mineral oil.

Another embodiment comprises cyclosporin A, Pluronic L43®, and Mineral oil.

Another embodiment comprises cyclosporin A, Pluronic L44®, and Mineral oil.

Another embodiment comprises cyclosporin A, Pluronic L61®, and Mineral oil.

Another embodiment comprises cyclosporin A, Pluronic L62®, and Mineral oil.

Another embodiment comprises cyclosporin A, Pluronic L62®, and Mineral oil.

Another embodiment comprises cyclosporin A, Pluronic L62LF®, and Mineral oil.

Another embodiment comprises cyclosporin A, Pluronic L64®, and Mineral oil.

Another embodiment comprises cyclosporin A, Pluronic L81®, and Mineral oil.

Another embodiment comprises cyclosporin A, Pluronic L92®, and Mineral oil.

Another embodiment comprises cyclosporin A, Pluronic P103®, and Mineral oil.

Another embodiment comprises cyclosporin A, Pluronic P104®, and Mineral oil.

Another embodiment comprises cyclosporin A, Pluronic P105®, and Mineral oil.

Another embodiment comprises cyclosporin A, Pluronic P123®, and Mineral oil.

Another embodiment comprises cyclosporin A, Pluronic P65®, and Mineral oil.

Another embodiment comprises cyclosporin A, Pluronic P84®, and Mineral oil.

Another embodiment comprises cyclosporin A, Pluronic P85®, and Mineral oil.

Another embodiment comprises cyclosporin A, Polyaldo 10-2-P®, and Mineral oil.

Another embodiment comprises cyclosporin A, Polyaldo DGDO®, and Mineral oil.

Another embodiment comprises cyclosporin A, Polyaldo HGDS®, and Mineral oil.

Another embodiment comprises cyclosporin A, Polyaldo TGMS®, and Mineral oil.

Another embodiment comprises cyclosporin A, Protegin V®, and Mineral oil.

Another embodiment comprises cyclosporin A, Protegin W®, and Mineral oil.

Another embodiment comprises cyclosporin A, Protegin WX®, and Mineral oil.

Another embodiment comprises cyclosporin A, Protegin XV®, and Mineral oil.

Another embodiment comprises cyclosporin A, Rhodasurf BC-420®, and Mineral oil.

Another embodiment comprises cyclosporin A, Rhodasurf BC-610®, and Mineral oil.

Another embodiment comprises cyclosporin A, Rhodasurf BC-630®, and Mineral oil.

Another embodiment comprises cyclosporin A, Rhodasurf BC-720®, and Mineral oil.

Another embodiment comprises cyclosporin A, Rhodasurf BC-840®, and Mineral oil.

Another embodiment comprises cyclosporin A, Rhodasurf L 1®, and Mineral oil.

Another embodiment comprises cyclosporin A, Rhodasurf L 12®, and Mineral oil.

Another embodiment comprises cyclosporin A, Rhodasurf L 2®, and Mineral oil.

Another embodiment comprises cyclosporin A, Rhodasurf L 20®, and Mineral oil.

Another embodiment comprises cyclosporin A, Rhodasurf L 3®, and Mineral oil.

Another embodiment comprises cyclosporin A, Rhodasurf L 30®, and Mineral oil.

Another embodiment comprises cyclosporin A, Rhodasurf L 4®, and Mineral oil.

Another embodiment comprises cyclosporin A, Rhodasurf L 50®, and Mineral oil.

Another embodiment comprises cyclosporin A, Rhodasurf L 7/90®, and Mineral oil.

Another embodiment comprises cyclosporin A, Rhodasurf LA-3®, and Mineral oil.

Another embodiment comprises cyclosporin A, Rhodasurf LA-7®, and Mineral oil.

Another embodiment comprises cyclosporin A, Rhodasurf LAN-23®, and Mineral oil.

Another embodiment comprises cyclosporin A, Rhodasurf ON-870®, and Mineral oil.

Another embodiment comprises cyclosporin A, Rhodasurf ON-877®, and Mineral oil.

Another embodiment comprises cyclosporin A, Rhodasurf TDA-8/5®, and Mineral oil.

Another embodiment comprises cyclosporin A, Ritapro 100®, and Mineral oil.

Another embodiment comprises cyclosporin A, Ritapro 165®, and Mineral oil.

Another embodiment comprises cyclosporin A, Ritapro 200®, and Mineral oil.

Another embodiment comprises cyclosporin A, Ritapro 300®, and Mineral oil.

Another embodiment comprises cyclosporin A, Ritoleth 10®, and Mineral oil.

Another embodiment comprises cyclosporin A, Ritoleth 2®, and Mineral oil.

Another embodiment comprises cyclosporin A, Ritoleth 20®, and Mineral oil.

Another embodiment comprises cyclosporin A, Ritoleth 5®, and Mineral oil.

Another embodiment comprises cyclosporin A, Santone 10-10-0®, and Mineral oil.

Another embodiment comprises cyclosporin A, Santone 3-1-S XTR®, and Mineral oil.

Another embodiment comprises cyclosporin A, Santone 3-1 -SH®and Mineral oil.

Another embodiment comprises cyclosporin A, Santone 8-1 -O®, and Mineral oil.

Another embodiment comprises cyclosporin A, Solulan 16®, and Mineral oil.

Another embodiment comprises cyclosporin A, Solulan 75®, and Mineral oil.

Another embodiment comprises cyclosporin A, Solulan C-24®, and Mineral oil.

Another embodiment comprises cyclosporin A, Solulan L-575®, and Mineral oil.

Another embodiment comprises cyclosporin A, Standamox CAW®, and Mineral oil.

Another embodiment comprises cyclosporin A, Surfine AZI-A®, and Mineral oil.

Another embodiment comprises cyclosporin A, Surfonic CO-15®, and Mineral oil.

Another embodiment comprises cyclosporin A, Surfonic CO-25®, and Mineral oil.

Another embodiment comprises cyclosporin A, Surfonic CO-30®, and Mineral oil.

Another embodiment comprises cyclosporin A, Surfonic CO-36®, and Mineral oil.

Another embodiment comprises cyclosporin A, Surfonic CO-42®, and Mineral oil.

Another embodiment comprises cyclosporin A, Surfonic E400-MO®, and Mineral oil.

Another embodiment comprises cyclosporin A, Surfonic L 12-3®, and Mineral oil.

Another embodiment comprises cyclosporin A, Surfonic L 12-6®, and Mineral oil.

Another embodiment comprises cyclosporin A, Surfonic L 12-8®, and Mineral oil.

Another embodiment comprises cyclosporin A, Surfonic L24-12®, and Mineral oil.

Another embodiment comprises cyclosporin A, Surfonic L24-2®, and Mineral oil.

Another embodiment comprises cyclosporin A, Surfonic L24-22®, and Mineral oil.

Another embodiment comprises cyclosporin A, Surfonic L24-3®, and Mineral oil.

Another embodiment comprises cyclosporin A, Surfonic L24-4®, and Mineral oil.

Another embodiment comprises cyclosporin A, Surfonic L24-5®, and Mineral oil.

Another embodiment comprises cyclosporin A, Surfonic L24-7®, and Mineral oil.

Another embodiment comprises cyclosporin A, Surfonic L24-7.1®, and Mineral oil.

Another embodiment comprises cyclosporin A, Surfonic L24-9®, and Mineral oil.

Another embodiment comprises cyclosporin A, Surfonic LF-17®, and Mineral oil.

Another embodiment comprises cyclosporin A, Surfonic LF-18®, and Mineral oil.

Another embodiment comprises cyclosporin A, Surfonic LF-37®, and Mineral oil.

Another embodiment comprises cyclosporin A, Surfonic LF-41®, and Mineral oil.

Another embodiment comprises cyclosporin A, Surfonic LF-42®, and Mineral oil.

Another embodiment comprises cyclosporin A, Surfonic LF-47®, and Mineral oil.

Another embodiment comprises cyclosporin A, Surfonic LF-68®, and Mineral oil.

Another embodiment comprises cyclosporin A, Surfonic N-10®, and Mineral oil.

Another embodiment comprises cyclosporin A, Surfonic N-100®, and Mineral oil.

Another embodiment comprises cyclosporin A, Surfonic N-102®, and Mineral oil.

Another embodiment comprises cyclosporin A, Surfonic N-120®, and Mineral oil.

Another embodiment comprises cyclosporin A, Surfonic N-150®, and Mineral oil.

Another embodiment comprises cyclosporin A, Surfonic N-200®, and Mineral oil.

Another embodiment comprises cyclosporin A, Surfonic N-300®, and Mineral oil.

Another embodiment comprises cyclosporin A, Surfonic N-31.5®, and Mineral oil.

Another embodiment comprises cyclosporin A, Surfonic N-40®, and Mineral oil.

Another embodiment comprises cyclosporin A, Surfonic N-400®, and Mineral oil.

Another embodiment comprises cyclosporin A, Surfonic N-60®, and Mineral oil.

Another embodiment comprises cyclosporin A, Surfonic N-85®, and Mineral oil.

Another embodiment comprises cyclosporin A, Surfonic N-95®, and Mineral oil.

Another embodiment comprises cyclosporin A, Surfonic NB-18®, and Mineral oil.

Another embodiment comprises cyclosporin A, Surfonic NB-23®, and Mineral oil.

Another embodiment comprises cyclosporin A, Surfonic NB-25®, and Mineral oil.

Another embodiment comprises cyclosporin A, Surfonic NB-307®, and Mineral oil.

Another embodiment comprises cyclosporin A, Surfonic NB-31®, and Mineral oil.

Another embodiment comprises cyclosporin A, Surfonic NB-41®, and Mineral oil.

Another embodiment comprises cyclosporin A, Surfonic NB-43®, and Mineral oil.

Another embodiment comprises cyclosporin A, Surfonic OP-100®, and Mineral oil.

Another embodiment comprises cyclosporin A, Surfonic OP-120®, and Mineral oil.

Another embodiment comprises cyclosporin A, Surfonic OP-15®, and Mineral oil.

Another embodiment comprises cyclosporin A, Surfonic OP-35®, and Mineral oil.

Another embodiment comprises cyclosporin A, Surfonic OP-70®, and Mineral oil.

Another embodiment comprises cyclosporin A, Surfonic P1®, and Mineral oil.

Another embodiment comprises cyclosporin A, Surfonic P3®, and Mineral oil.

Another embodiment comprises cyclosporin A, Surfonic P5®, and Mineral oil.

Another embodiment comprises cyclosporin A, Surfonic P6®, and Mineral oil.

Another embodiment comprises cyclosporin A, Surfonic POA-17R2®, and Mineral oil.

Another embodiment comprises cyclosporin A, Surfonic POA-25R2®, and Mineral oil.

Another embodiment comprises cyclosporin A, Surfonic TDA-11®, and Mineral oil.

Another embodiment comprises cyclosporin A, Surfonic TDA-3B®, and Mineral oil.

Another embodiment comprises cyclosporin A, Surfonic TDA-6®, and Mineral oil.

Another embodiment comprises cyclosporin A, Surfonic TDA-6/88®, and Mineral oil.

Another embodiment comprises cyclosporin A, Surfonic TDA-8®, and Mineral oil.

Another embodiment comprises cyclosporin A, Surfonic TDA-8/90®, and Mineral oil.

Another embodiment comprises cyclosporin A, Surfonic TDA-9®, and Mineral oil.

Another embodiment comprises cyclosporin A, Surfonic TX-CCN®, and Mineral oil.

Another embodiment comprises cyclosporin A, Tagat CH 40®, and Mineral oil.

Another embodiment comprises cyclosporin A, Tagat CH 60®, and Mineral oil.

Another embodiment comprises cyclosporin A, Tagat L 2®, and Mineral oil.

Another embodiment comprises cyclosporin A, Tagat S®, and Mineral oil.

Another embodiment comprises cyclosporin A, Tagat S 2®, and Mineral oil.

Another embodiment comprises cyclosporin A, Tally 100 Plus®, and Mineral oil.

Another embodiment comprises cyclosporin A, T-Det A106®, and Mineral oil.

Another embodiment comprises cyclosporin A, T-Det A109®, and Mineral oil.

Another embodiment comprises cyclosporin A, T-Det A136®, and Mineral oil.

Another embodiment comprises cyclosporin A, T-Det A139®, and Mineral oil.

Another embodiment comprises cyclosporin A, T-Det A2412®, and Mineral oil.

Another embodiment comprises cyclosporin A, T-Det A243®, and Mineral oil.

Another embodiment comprises cyclosporin A, T-Det A247®, and Mineral oil.

Another embodiment comprises cyclosporin A, T-Det A249®, and Mineral oil.

Another embodiment comprises cyclosporin A, T-Det A467®, and Mineral oil.

Another embodiment comprises cyclosporin A, T-Det COE®, and Mineral oil.

Another embodiment comprises cyclosporin A, T-Det DD-10®, and Mineral oil.

Another embodiment comprises cyclosporin A, T-Det.DD-5®, and Mineral oil.

Another embodiment comprises cyclosporin A, T-Det DD-7®, and Mineral oil.

Another embodiment comprises cyclosporin A, T-Det EPO-104®, and Mineral oil.

Another embodiment comprises cyclosporin A, T-Det EPO-61®, and Mineral oil.

Another embodiment comprises cyclosporin A, T-Det EPO-62®, and Mineral oil.

Another embodiment comprises cyclosporin A, T-Det EPO-64®, and Mineral oil.

Another embodiment comprises cyclosporin A, T-Det N-1.5®, and Mineral oil.

Another embodiment comprises cyclosporin A, T-Det N-10.5®, and Mineral oil.

Another embodiment comprises cyclosporin A, T-Det N-100®, and Mineral oil.

Another embodiment comprises cyclosporin A, T-Det N-1007®, and Mineral oil.

Another embodiment comprises cyclosporin A, T-Det N-12®, and Mineral oil.

Another embodiment comprises cyclosporin A, T-Det N-14®, and Mineral oil.

Another embodiment comprises cyclosporin A, T-Det N-20®, and Mineral oil.

Another embodiment comprises cyclosporin A, T-Det N-30®, and Mineral oil.

Another embodiment comprises cyclosporin A, T-Det N-307®, and Mineral oil.

Another embodiment comprises cyclosporin A, T-Det N-4®, and Mineral oil.

Another embodiment comprises cyclosporin A, T-Det N-40®, and Mineral oil.

Another embodiment comprises cyclosporin A, T-Det N-407®, and Mineral oil.

Another embodiment comprises cyclosporin A, T-Det N-50®, and Mineral oil.

Another embodiment comprises cyclosporin A, T-Det N-507®, and Mineral oil.

Another embodiment comprises cyclosporin A, T-Det N-6®, and Mineral oil.

Another embodiment comprises cyclosporin A, T-Det N-70®, and Mineral oil.

Another embodiment comprises cyclosporin A, T-Det N-8®, and Mineral oil.

Another embodiment comprises cyclosporin A, T-Det N-9.5®, and Mineral oil.

Another embodiment comprises cyclosporin A, T-Det O-12®, and Mineral oil.

Another embodiment comprises cyclosporin A, T-Det O-165®, and Mineral oil.

Another embodiment comprises cyclosporin A, T-Det O-307®, and Mineral oil.

Another embodiment comprises cyclosporin A, T-Det O-40®, and Mineral oil.

Another embodiment comprises cyclosporin A, T-Det O-407®, and Mineral oil.

Another embodiment comprises cyclosporin A, T-Det O-6®, and Mineral oil.

Another embodiment comprises cyclosporin A, T-Det O-8®, and Mineral oil.

Another embodiment comprises cyclosporin A, T-Det O-9®, and Mineral oil.

Another embodiment comprises cyclosporin A, Tegin®, and Mineral oil.

Another embodiment comprises cyclosporin A, Tergitol 15-S-12®, and Mineral oil.

Another embodiment comprises cyclosporin A, Tergitol 15-S-15®, and Mineral oil.

Another embodiment comprises cyclosporin A, Tergitol 15-S-20®, and Mineral oil.

Another embodiment comprises cyclosporin A, Tergitol 15-S-20 (8AQ)®, and Mineral oil.

Another embodiment comprises cyclosporin A, Tergitol 15-S-3®, and Mineral oil.

Another embodiment comprises cyclosporin A, Tergitol 15-S-30®, and Mineral oil.

Another embodiment comprises cyclosporin A, Tergitol 15-S-40®, and Mineral oil.

Another embodiment comprises cyclosporin A, Tergitol 15-S-40 (70AQ)®, and Mineral oil.

Another embodiment comprises cyclosporin A, Tergitol 15-S-5®, and Mineral oil.

Another embodiment comprises cyclosporin A, Tergitol 15-S-7®, and Mineral oil.

Another embodiment comprises cyclosporin A, Tergitol 15-S-9®, and Mineral oil.

Another embodiment comprises cyclosporin A, Tergitol L-101®, and Mineral oil.

Another embodiment comprises cyclosporin A, Tergitol L-61®, and Mineral oil.

Another embodiment comprises cyclosporin A, Tergitol L-62®, and Mineral oil.

Another embodiment comprises cyclosporin A, Tergitol L-64®, and Mineral oil.

Another embodiment comprises cyclosporin A, Tergitol L-81®, and Mineral oil.

Another embodiment comprises cyclosporin A, Tergitol NP-10®, and Mineral oil.

Another embodiment comprises cyclosporin A, Tergitol NP-11®, and Mineral oil.

Another embodiment comprises cyclosporin A, Tergitol NP-12®, and Mineral oil.

Another embodiment comprises cyclosporin A, Tergitol NP-13®, and Mineral oil.

Another embodiment comprises cyclosporin A, Tergitol NP-15®, and Mineral oil.

Another embodiment comprises cyclosporin A, Tergitol NP-30®, and Mineral oil.

Another embodiment comprises cyclosporin A, Tergitol NP-30 (70 AQ)®, and Mineral oil.

Another embodiment comprises cyclosporin A, Tergitol NP-4®, and Mineral oil.

Another embodiment comprises cyclosporin A, Tergitol NP-40®, and Mineral oil.

Another embodiment comprises cyclosporin A, Tergitol NP-40 (70 AQ)®, and Mineral oil.

Another embodiment comprises cyclosporin A, Tergitol NP-50 (70 AQ)®, and Mineral oil.

Another embodiment comprises cyclosporin A, Tergitol NP-55 (70 AQ)®, and Mineral oil.

Another embodiment comprises cyclosporin A, Tergitol NP-6®, and Mineral oil.

Another embodiment comprises cyclosporin A, Tergitol NP-7®, and Mineral oil.

Another embodiment comprises cyclosporin A, Tergitol NP-70 (70 AQ)®, and Mineral oil.

Another embodiment comprises cyclosporin A, Tergitol NP-8®, and Mineral oil.

Another embodiment comprises cyclosporin A, Tergitol NP-9®, and Mineral oil.

Another embodiment comprises cyclosporin A, Tergitol NP-9.5®, and Mineral oil.

Another embodiment comprises cyclosporin A, Tergitol TMN-10®, and Mineral oil.

Another embodiment comprises cyclosporin A, Tergitol TMN-3®, and Mineral oil.

Another embodiment comprises cyclosporin A, Tergitol TMN-6®, and Mineral oil.

Another embodiment comprises cyclosporin A, Tergitol XD®, and Mineral oil.

Another embodiment comprises cyclosporin A, Tergitol XH®, and Mineral oil.

Another embodiment comprises cyclosporin A, Tergitol XJ®, and Mineral oil.

Another embodiment comprises cyclosporin A, Tetronic 1107®, and Mineral oil.

Another embodiment comprises cyclosporin A, Tetronic 1307®, and Mineral oil.

Another embodiment comprises cyclosporin A, Tetronic 150R10®, and Mineral oil.

Another embodiment comprises cyclosporin A, Tetronic 304®, and Mineral oil.

Another embodiment comprises cyclosporin A, Tetronic 701®, and Mineral oil.

Another embodiment comprises cyclosporin A, Tetronic 901®, and Mineral oil.

Another embodiment comprises cyclosporin A, Tetronic 904®, and Mineral oil.

Another embodiment comprises cyclosporin A, Tetronic 908s, and Mineral oil.

Another embodiment comprises cyclosporin A, Tetronic 90R4®, and Mineral oil.

Another embodiment comprises cyclosporin A, Tomadol 1-3®, and Mineral oil.

Another embodiment comprises cyclosporin A, Tomadol 1-5®, and Mineral oil.

Another embodiment comprises cyclosporin A, Tomadol 1-7®, and Mineral oil.

Another embodiment comprises cyclosporin A, Tomadol 23-1®, and Mineral oil.

Another embodiment comprises cyclosporin A, Tomadol 23-3®, and Mineral oil.

Another embodiment comprises cyclosporin A, Tomadol 23-5®, and Mineral oil.

Another embodiment comprises cyclosporin A, Tomadol 23-6.5®, and Mineral oil.

Another embodiment comprises cyclosporin A, Tomadol 25-12®, and Mineral oil.

Another embodiment comprises cyclosporin A, Tomadol 25-3®, and Mineral oil.

Another embodiment comprises cyclosporin A, Tomadol 25-7®, and Mineral oil.

Another embodiment comprises cyclosporin A, Tomadol 25-9®, and Mineral oil.

Another embodiment comprises cyclosporin A, Tomadol 45-13®, and Mineral oil.

Another embodiment comprises cyclosporin A, Tomadol 45-2.5®, and Mineral oil.

Another embodiment comprises cyclosporin A, Tomadol 45-7®, and Mineral oil.

Another embodiment comprises cyclosporin A, Tomadol 91-2.5®, and Mineral oil.

Another embodiment comprises cyclosporin A, Tomadol 91-6®, and Mineral oil.

Another embodiment comprises cyclosporin A, Tomadol 91-8®, and Mineral oil.

Another embodiment comprises cyclosporin A, Triton X-100®, and Mineral oil.

Another embodiment comprises cyclosporin A, Triton X-102®, and Mineral oil.

Another embodiment comprises cyclosporin A, Triton X-114®, and Mineral oil.

Another embodiment comprises cyclosporin A, Triton X-15®, and Mineral oil.

Another embodiment comprises cyclosporin A, Triton X-165 (70%)®, and Mineral oil.

Another embodiment comprises cyclosporin A, Triton X-207®, and Mineral oil.

Another embodiment comprises cyclosporin A, Triton X-305 (70%)®, and Mineral oil.

Another embodiment comprises cyclosporin A, Triton X-35®, and Mineral oil.

Another embodiment comprises cyclosporin A, Triton X-405 (70%)®, and Mineral oil.

Another embodiment comprises cyclosporin A, Triton X-45®, and Mineral oil.

Another embodiment comprises cyclosporin A, Triton X-705 (70%)®, and Mineral oil.

Another embodiment comprises cyclosporin A, Volpo S-10®, and Mineral oil.

Another embodiment comprises cyclosporin A, Volpo S-2®, and Mineral oil.

Another embodiment comprises cyclosporin A, Volpo S-20®, and Mineral oil.

Another embodiment comprises cyclosporin A, Volpo-10®, and Mineral oil.

Another embodiment comprises cyclosporin A, Volpo-20®, and Mineral oil.

Another embodiment comprises cyclosporin A, Volpo-3®, and Mineral oil.

Another embodiment comprises cyclosporin A, Volpo-5®, and Mineral oil.

Another embodiment comprises cyclosporin A, Abex 2515®, and Coriander oil.

Another embodiment comprises cyclosporin A, Abex 2525/40®, and Coriander oil.

Another embodiment comprises cyclosporin A, Abex 2535®, and Coriander oil.

Another embodiment comprises cyclosporin A, Abex 2545®, and Coriander oil.

Another embodiment comprises cyclosporin A, Acconon E®, and Coriander oil.

Another embodiment comprises cyclosporin A, Alfonic 1216CO-9®, and Coriander oil.

Another embodiment comprises cyclosporin A, Alfonic 1412-3®, and Coriander oil.

Another embodiment comprises cyclosporin A, Alfonic TDA-12®, and Coriander oil.

Another embodiment comprises cyclosporin A, Alfonic TDA-3®, and Coriander oil.

Another embodiment comprises cyclosporin A, Alfonic TDA-4®, and Coriander oil.

Another embodiment comprises cyclosporin A, Alfonic TDA-6®, and Coriander oil.

Another embodiment comprises cyclosporin A, Alfonic TDA-7®, and Coriander oil.

Another embodiment comprises cyclosporin A, Alfonic TDA-8®, and Coriander oil.

Another embodiment comprises cyclosporin A, Alfonic TDA-9®, and Coriander oil.

Another embodiment comprises cyclosporin A, Alkamuls 400-DO®, and Coriander oil.

Another embodiment comprises cyclosporin A, Alkamuls 400-MO/E®, and Coriander oil.

Another embodiment comprises cyclosporin A, Ameroxol OE-10®, and Coriander oil.

Another embodiment comprises cyclosporin A, Ameroxol OE-2®, and Coriander oil.

Another embodiment comprises cyclosporin A, Ameroxol OE-20®, and Coriander oil.

Another embodiment comprises cyclosporin A, Armix 176®, and Coriander oil.

Another embodiment comprises cyclosporin A, Armix 180-C®, and Coriander oil.

Another embodiment comprises cyclosporin A, Armix 183®, and Coriander oil.

Another embodiment comprises cyclosporin A, Brij 35®, and Coriander oil.

Another embodiment comprises cyclosporin A, Brij 35 Liquid®, and Coriander oil.

Another embodiment comprises cyclosporin A, Brij 35 SP®, and Coriander oil.

Another embodiment comprises cyclosporin A, Canasol MJ 2109®, and Coriander oil.

Another embodiment comprises cyclosporin A, Canasol MJ 52®, and Coriander oil.

Another embodiment comprises cyclosporin A, Canasol R 3600®, and Coriander oil.

Another embodiment comprises cyclosporin A, Canasol R 3603®, and Coriander oil.

Another embodiment comprises cyclosporin A, Canasol R 4000 H®, and Coriander oil.

Another embodiment comprises cyclosporin A, Cedepal CA-210®, and Coriander oil.

Another embodiment comprises cyclosporin A, Cedepal CA-520®, and Coriander oil.

Another embodiment comprises cyclosporin A, Cedepal CA-630®, and Coriander oil.

Another embodiment comprises cyclosporin A, Cedepal CA-720®, and Coriander oil.

Another embodiment comprises cyclosporin A, Cedepal CA-890®, and Coriander oil.

Another embodiment comprises cyclosporin A, Cedepal CA-897®, and Coriander oil.

Another embodiment comprises cyclosporin A, Cedepal CO-210®, and Coriander oil.

Another embodiment comprises cyclosporin A, Cedepal CO-430®, and Coriander oil.

Another embodiment comprises cyclosporin A, Cedepal CO-530®, and Coriander oil.

Another embodiment comprises cyclosporin A, Cedepal CO-630®, and Coriander oil.

Another embodiment comprises cyclosporin A, Cedepal CO-710®, and Coriander oil.

Another embodiment comprises cyclosporin A, Cedepal CO-730®, and Coriander oil.

Another embodiment comprises cyclosporin A, Cedepal CO-880®, and Coriander oil.

Another embodiment comprises cyclosporin A, Cedepal CO-887®, and Coriander oil.

Another embodiment comprises cyclosporin A, Cedepal CO-890®, and Coriander oil.

Another embodiment comprises cyclosporin A, Cedepal CO-897®, and Coriander oil.

Another embodiment comprises cyclosporin A, Cedepal CO-970®, and Coriander oil.

Another embodiment comprises cyclosporin A, Cedepal CO-977®, and Coriander oil.

Another embodiment comprises cyclosporin A, Cedepal CO-990®, and Coriander oil.

Another embodiment comprises cyclosporin A, Cedepal CO-997®, and Coriander oil.

Another embodiment comprises cyclosporin A, Chemal BP 261®, and Coriander oil.

Another embodiment comprises cyclosporin A, Chemal BP 262®, and Coriander oil.

Another embodiment comprises cyclosporin A, Chemal BP 3172®, and Coriander oil.

Another embodiment comprises cyclosporin A, Chemal BP 3174®, and Coriander oil.

Another embodiment comprises cyclosporin A, Chemal DA-4®, and Coriander oil.

Another embodiment comprises cyclosporin A, Chemal DA-6®, and Coriander oil.

Another embodiment comprises cyclosporin A, Chemal DA-9®, and Coriander oil.

Another embodiment comprises cyclosporin A, Chemal LA-23®, and Coriander oil.

Another embodiment comprises cyclosporin A, Chemal LA-4®, and Coriander oil.

Another embodiment comprises cyclosporin A, Chemal LA-9®, and Coriander oil.

Another embodiment comprises cyclosporin A, Chemal OA-20G®, and Coriander oil.

Another embodiment comprises cyclosporin A, Chemal OA-5®, and Coriander oil.

Another embodiment comprises cyclosporin A, Chemal OA-9®, and Coriander oil.

Another embodiment comprises cyclosporin A, Chemal TDA-12®, and Coriander oil.

Another embodiment comprises cyclosporin A, Chemal TDA-15®, and Coriander oil.

Another embodiment comprises cyclosporin A, Chemal TDA-3®, and Coriander oil.

Another embodiment comprises cyclosporin A, Chemal TDA-6®, and Coriander oil.

Another embodiment comprises cyclosporin A, Chemal TDA-9®, and Coriander oil.

Another embodiment comprises cyclosporin A, Chemax CO-16®, and Coriander oil.

Another embodiment comprises cyclosporin A, Chemax CO-200/50®, and Coriander oil.

Another embodiment comprises cyclosporin A, Chemax CO-25®, and Coriander oil.

Another embodiment comprises cyclosporin A, Chemax CO-30®, and Coriander oil.

Another embodiment comprises cyclosporin A, Chemax CO-36®, and Coriander oil.

Another embodiment comprises cyclosporin A, Chemax CO-40®, and Coriander oil.

Another embodiment comprises cyclosporin A, Chemax CO-5®, and Coriander oil.

Another embodiment comprises cyclosporin A, Chemax CO-80®, and Coriander oil.

Another embodiment comprises cyclosporin A, Chemax DNP-150/50®, and Coriander oil.

Another embodiment comprises cyclosporin A, Chemax DNP-18®, and Coriander oil.

Another embodiment comprises cyclosporin A, Chemax DNP-8®, and Coriander oil.

Another embodiment comprises cyclosporin A, Chemax E 200 ML®, and Coriander oil.

Another embodiment comprises cyclosporin A, Chemax E 200 MO®, and Coriander oil.

Another embodiment comprises cyclosporin A, Chemax E 400 ML®, and Coriander oil.

Another embodiment comprises cyclosporin A, Chemax E 400 MO®, and Coriander oil.

Another embodiment comprises cyclosporin A, Chemax E 600 ML®, and Coriander oil.

Another embodiment comprises cyclosporin A, Chemax E 600 MO®, and Coriander oil.

Another embodiment comprises cyclosporin A, Chemax E1000 MO®, and Coriander oil.

Another embodiment comprises cyclosporin A, Chemax E1000 MS®, and Coriander oil.

Another embodiment comprises cyclosporin A, Chemax E200 MS®, and Coriander oil.

Another embodiment comprises cyclosporin A, Chemax E400 MS®, and Coriander oil.

Another embodiment comprises cyclosporin A, Chemax E600 MS®, and Coriander oil.

Another embodiment comprises cyclosporin A, Chemax HCO-16®, and Coriander oil.

Another embodiment comprises cyclosporin A, Chemax HCO-200/50®, and Coriander oil.

Another embodiment comprises cyclosporin A, Chemax HCO-25®, and Coriander oil.

Another embodiment comprises cyclosporin A, Chemax HCO-5®, and Coriander oil.

Another embodiment comprises cyclosporin A, Chemax TO-16®, and Coriander oil.

Another embodiment comprises cyclosporin A, Chemax TO-8®, and Coriander oil.

Another embodiment comprises cyclosporin A, Colalube 3409®, and Coriander oil.

Another embodiment comprises cyclosporin A, Colalube 3411®, and Coriander oil.

Another embodiment comprises cyclosporin A, Colalube 3413®, and Coriander oil.

Another embodiment comprises cyclosporin A, Colalube 3414®, and Coriander oil.

Another embodiment comprises cyclosporin A, Colalube 3415®, and Coriander oil.

Another embodiment comprises cyclosporin A, Colalube 3416®, and Coriander oil.

Another embodiment comprises cyclosporin A, Colalube 3417®, and Coriander oil.

Another embodiment comprises cyclosporin A, Delonic NPE-100®, and Coriander oil.

Another embodiment comprises cyclosporin A, Delonic NPE-30®, and Coriander oil.

Another embodiment comprises cyclosporin A, Delonic NPE-40®, and Coriander oil.

Another embodiment comprises cyclosporin A, Delonic OPE-10®, and Coriander oil.

Another embodiment comprises cyclosporin A, Delonic OPE-12®, and Coriander oil.

Another embodiment comprises cyclosporin A, Delonic OPE-30®, and Coriander oil.

Another embodiment comprises cyclosporin A, Delonic OPE-40®, and Coriander oil.

Another embodiment comprises cyclosporin A, Delonic OPE-5®, and Coriander oil.

Another embodiment comprises cyclosporin A, Delonic OPE-7.5®, and Coriander oil.

Another embodiment comprises cyclosporin A, DePeg 16-CO®, and Coriander oil.

Another embodiment comprises cyclosporin A, DePeg 25-CO®, and Coriander oil.

Another embodiment comprises cyclosporin A, DePeg 30-CO®, and Coriander oil.

Another embodiment comprises cyclosporin A, DePeg 40-CO®, and Coriander oil.

Another embodiment comprises cyclosporin A, DePeg 5-CO®, and Coriander oil.

Another embodiment comprises cyclosporin A, DePeg 80-CO®, and Coriander oil.

Another embodiment comprises cyclosporin A, DeSonic S-100®, and Coriander oil.

Another embodiment comprises cyclosporin A, DeSonic S-114®, and Coriander oil.

Another embodiment comprises cyclosporin A, DeSonic S-405®, and Coriander oil.

Another embodiment comprises cyclosporin A, DeSonic S-45®, and Coriander oil.

Another embodiment comprises cyclosporin A, DeSonic TDA-9®, and Coriander oil.

Another embodiment comprises cyclosporin A, DeThox Acid O-14®, and Coriander oil.

Another embodiment comprises cyclosporin A, DeThox Acid O-16®, and Coriander oil.

Another embodiment comprises cyclosporin A, DeThox Acid O-9®, and Coriander oil.

Another embodiment comprises cyclosporin A, DeThox Acid TO-14®, and Coriander oil.

Another embodiment comprises cyclosporin A, DeThox Acid TO-16.5®, and Coriander oil.

Another embodiment comprises cyclosporin A, DeThox Acid TO-8.5®, and Coriander oil.

Another embodiment comprises cyclosporin A, Dur-Em 114®, and Coriander oil.

Another embodiment comprises cyclosporin A, Dur-Em 117®, and Coriander oil.

Another embodiment comprises cyclosporin A, Dur-Em 204®, and Coriander oil.

Another embodiment comprises cyclosporin A, Dur-Em 207®, and Coriander oil.

Another embodiment comprises cyclosporin A, Dur-Em 207E®, and Coriander oil.

Another embodiment comprises cyclosporin A, Emerest 270®, and Coriander oil.

Another embodiment comprises cyclosporin A, Emerest 2712®, and Coriander oil.

Another embodiment comprises cyclosporin A, Emerest 2715®, and Coriander oil.

Another embodiment comprises cyclosporin A, Ethal EH-2®, and Coriander oil.

Another embodiment comprises cyclosporin A, Ethal EH-5®, and Coriander oil.

Another embodiment comprises cyclosporin A, Ethal OA-23®, and Coriander oil.

Another embodiment comprises cyclosporin A, Ethosperse CA-20®, and Coriander oil.

Another embodiment comprises cyclosporin A, Ethosperse LA-12®, and Coriander oil.

Another embodiment comprises cyclosporin A, Ethosperse LA-23®, and Coriander oil.

Another embodiment comprises cyclosporin A, Ethosperse LA-4®, and Coriander oil.

Another embodiment comprises cyclosporin A, Ethox 3095®, and Coriander oil.

Another embodiment comprises cyclosporin A, Ethox CO-16®, and Coriander oil.

Another embodiment comprises cyclosporin A, Ethox CO-200®, and Coriander oil.

Another embodiment comprises cyclosporin A, Ethox CO-25®, and Coriander oil.

Another embodiment comprises cyclosporin A, Ethox CO-30®, and Coriander oil.

Another embodiment comprises cyclosporin A, Ethox CO-36®, and Coriander oil.

Another embodiment comprises cyclosporin A, Ethox CO-40®, and Coriander oil.

Another embodiment comprises cyclosporin A, Ethox CO-5®, and Coriander oil.

Another embodiment comprises cyclosporin A, Ethox DO-14®, and Coriander oil.

Another embodiment comprises cyclosporin A, Ethox DO-9®, and Coriander oil.

Another embodiment comprises cyclosporin A, Ethox MA-8®, and Coriander oil.

Another embodiment comprises cyclosporin A, Ethox MI-14®, and Coriander oil.

Another embodiment comprises cyclosporin A, Ethox ML-14®, and Coriander oil.

Another embodiment comprises cyclosporin A, Ethox ML-5®, and Coriander oil.

Another embodiment comprises cyclosporin A, Ethox MO-14®, and Coriander oil.

Another embodiment comprises cyclosporin A, Ethox MO-9®, and Coriander oil.

Another embodiment comprises cyclosporin A, Ethox MS-14®, and Coriander oil.

Another embodiment comprises cyclosporin A, Ethox MS-23®, and Coriander oil.

Another embodiment comprises cyclosporin A, Ethox MS-40®, and Coriander oil.

Another embodiment comprises cyclosporin A, Ethox MS-8®, and Coriander oil.

Another embodiment comprises cyclosporin A, Ethox TO-16®, and Coriander oil.

Another embodiment comprises cyclosporin A, Ethox TO-8®, and Coriander oil.

Another embodiment comprises cyclosporin A, Ethylan SN-120®, and Coriander oil.

Another embodiment comprises cyclosporin A, Ethylan SN-70®, and Coriander oil.

Another embodiment comprises cyclosporin A, Ethylan SN-90®, and Coriander oil.

Another embodiment comprises cyclosporin A, Flexricin 13®, and Coriander oil.

Another embodiment comprises cyclosporin A, Flexricin 15®, and Coriander oil.

Another embodiment comprises cyclosporin A, Genapol 1261®, and Coriander oil.

Another embodiment comprises cyclosporin A, Generol 122 E25®, and Coriander oil.

Another embodiment comprises cyclosporin A, Glicepol 160®, and Coriander oil.

Another embodiment comprises cyclosporin A, Glicepol 560®, and Coriander oil.

Another embodiment comprises cyclosporin A, Glicepol GMS 20®, and Coriander oil.

Another embodiment comprises cyclosporin A, Hetoxide BN-13®, and Coriander oil.

Another embodiment comprises cyclosporin A, Hetoxide DNP-4®, and Coriander oil.

Another embodiment comprises cyclosporin A, Hetoxide DNP-9.6®, and Coriander oil.

Another embodiment comprises cyclosporin A, Hetoxide G-26®, and Coriander oil.

Another embodiment comprises cyclosporin A, Hetoxide G-7®, and Coriander oil.

Another embodiment comprises cyclosporin A, Hetoxide GT-20®, and Coriander oil.

Another embodiment comprises cyclosporin A, Hetoxide HC-40®, and Coriander oil.

Another embodiment comprises cyclosporin A, Hetoxol C-24®, and Coriander oil.

Another embodiment comprises cyclosporin A, Hetoxol CA-10®, and Coriander oil.

Another embodiment comprises cyclosporin A, Hetoxol CA-2®, and Coriander oil.

Another embodiment comprises cyclosporin A, Hetoxol CA-20®, and Coriander oil.

Another embodiment comprises cyclosporin A, Hetoxol CAWS®, and Coriander oil.

Another embodiment comprises cyclosporin A, Hetoxol CD-3®, and Coriander oil.

Another embodiment comprises cyclosporin A, Hetoxol CD-4®, and Coriander oil.

Another embodiment comprises cyclosporin A, Hetoxol CS-15®, and Coriander oil.

Another embodiment comprises cyclosporin A, Hetoxol CS-20®, and Coriander oil.

Another embodiment comprises cyclosporin A, Hetoxol CS-20D®, and Coriander oil.

Another embodiment comprises cyclosporin A, Hetoxol CS-25®, and Coriander oil.

Another embodiment comprises cyclosporin A, Hetoxol CS-50®, and Coriander oil.

Another embodiment comprises cyclosporin A, Hetoxol CS-9®, and Coriander oil.

Another embodiment comprises cyclosporin A, Hetoxol CSA-15®, and Coriander oil.

Another embodiment comprises cyclosporin A, Hetoxol D®, and Coriander oil.

Another embodiment comprises cyclosporin A, Hetoxol G®, and Coriander oil.

Another embodiment comprises cyclosporin A, Hetoxol J®, and Coriander oil.

Another embodiment comprises cyclosporin A, Hetoxol L®, and Coriander oil.

Another embodiment comprises cyclosporin A, Hetoxol L-23®, and Coriander oil.

Another embodiment comprises cyclosporin A, Hetoxol L-4®, and Coriander oil.

Another embodiment comprises cyclosporin A, Hetoxol L-9®, and Coriander oil.

Another embodiment comprises cyclosporin A, Hetoxol LS-9®, and Coriander oil.

Another embodiment comprises cyclosporin A, Hetoxol M-3®, and Coriander oil.

Another embodiment comprises cyclosporin A, Hetoxol OA-10 Special®, and Coriander oil.

Another embodiment comprises cyclosporin A, Hetoxol OA-20 Special®, and Coriander oil.

Another embodiment comprises cyclosporin A, Hetoxol OA-3 Special®, and Coriander oil.

Another embodiment comprises cyclosporin A, Hetoxol OA-5 Special®, and Coriander oil.

Another embodiment comprises cyclosporin A, Hetoxol OL-10®, and Coriander oil.

Another embodiment comprises cyclosporin A, Hetoxol OL-2®, and Coriander oil.

Another embodiment comprises cyclosporin A, Hetoxol OL-23®, and Coriander oil.

Another embodiment comprises cyclosporin A, Hetoxol OL-4®, and Coriander oil.

Another embodiment comprises cyclosporin A, Hetoxol OL-40®, and Coriander oil.

Another embodiment comprises cyclosporin A, Hetoxol OL-5®, and Coriander oil.

Another embodiment comprises cyclosporin A, Hetoxol PLA®, and Coriander oil.

Another embodiment comprises cyclosporin A, Hetoxol SP-15®, and Coriander oil.

Another embodiment comprises cyclosporin A, Hetoxol STA-10®, and Coriander oil.

Another embodiment comprises cyclosporin A, Hetoxol STA-2®, and Coriander oil.

Another embodiment comprises cyclosporin A, Hetoxol STA-30®, and Coriander oil.

Another embodiment comprises cyclosporin A, Hetoxol TD-12®, and Coriander oil.

Another embodiment comprises cyclosporin A, Hetoxol TD-18®, and Coriander oil.

Another embodiment comprises cyclosporin A, Hetoxol TD-3®, and Coriander oil.

Another embodiment comprises cyclosporin A, Hetoxol TD-6®, and Coriander oil.

Another embodiment comprises cyclosporin A, Hetoxol TD-9®, and Coriander oil.

Another embodiment comprises cyclosporin A, Hetoxol TDEP-15®, and Coriander oil.

Another embodiment comprises cyclosporin A, Hetoxol TDEP-63®, and Coriander oil.

Another embodiment comprises cyclosporin A, Iconol 24-12®, and Coriander oil.

Another embodiment comprises cyclosporin A, Iconol 24-23®, and Coriander oil.

Another embodiment comprises cyclosporin A, Iconol 24-4®, and Coriander oil.

Another embodiment comprises cyclosporin A, Iconol DA-4®, and Coriander oil.

Another embodiment comprises cyclosporin A, Iconol DA-6®, and Coriander oil.

Another embodiment comprises cyclosporin A, Iconol TDA-10®, and Coriander oil.

Another embodiment comprises cyclosporin A, Iconol TDA-3®, and Coriander oil.

Another embodiment comprises cyclosporin A, Iconol TDA-6®, and Coriander oil.

Another embodiment comprises cyclosporin A, Iconol TDA-8-90%®, and Coriander oil.

Another embodiment comprises cyclosporin A, Iconol TDA-9®, and Coriander oil.

Another embodiment comprises cyclosporin A, Igepal CA-210®, and Coriander oil.

Another embodiment comprises cyclosporin A, Igepal CA-407®, and Coriander oil.

Another embodiment comprises cyclosporin A, Igepal CA-420®, and Coriander oil.

Another embodiment comprises cyclosporin A, Igepal CA-520®, and Coriander oil.

Another embodiment comprises cyclosporin A, Igepal CA-620®, and Coriander oil.

Another embodiment comprises cyclosporin A, Igepal CA-630®, and Coriander oil.

Another embodiment comprises cyclosporin A, Igepal CA-720®, and Coriander oil.

Another embodiment comprises cyclosporin A, Igepal CA-877®, and Coriander oil.

Another embodiment comprises cyclosporin A, Igepal CA-887®, and Coriander oil.

Another embodiment comprises cyclosporin A, Igepal CA-897®, and Coriander oil.

Another embodiment comprises cyclosporin A, Igepal CO-210®, and Coriander oil.

Another embodiment comprises cyclosporin A, Igepal CO-430®, and Coriander oil.

Another embodiment comprises cyclosporin A, Igepal CO-520®, and Coriander oil.

Another embodiment comprises cyclosporin A, Igepal CO-530®, and Coriander oil.

Another embodiment comprises cyclosporin A, Igepal CO-610®, and Coriander oil.

Another embodiment comprises cyclosporin A, Igepal CO-630®, and Coriander oil.

Another embodiment comprises cyclosporin A, Igepal CO-630 Special®, and Coriander oil.

Another embodiment comprises cyclosporin A, Igepal CO-660®, and Coriander oil.

Another embodiment comprises cyclosporin A, Igepal CO-710®, and Coriander oil.

Another embodiment comprises cyclosporin A, Igepal CO-720®, and Coriander oil.

Another embodiment comprises cyclosporin A, Igepal CO-730®, and Coriander oil.

Another embodiment comprises cyclosporin A, Igepal CO-738®, and Coriander oil.

Another embodiment comprises cyclosporin A, Igepal CO-850®, and Coriander oil.

Another embodiment comprises cyclosporin A, Igepal CO-880®, and Coriander oil.

Another embodiment comprises cyclosporin A, Igepal CO-887®, and Coriander oil.

Another embodiment comprises cyclosporin A, Igepal CO-890®, and Coriander oil.

Another embodiment comprises cyclosporin A, Igepal CO-897®, and Coriander oil.

Another embodiment comprises cyclosporin A, Igepal CO-970®, and Coriander oil.

Another embodiment comprises cyclosporin A, Igepal CO-977®, and Coriander oil.

Another embodiment comprises cyclosporin A, Igepal CO-987®, and Coriander oil.

Another embodiment comprises cyclosporin A, Igepal CO-990 FLK®, and Coriander oil.

Another embodiment comprises cyclosporin A, Igepal CO-997®, and Coriander oil.

Another embodiment comprises cyclosporin A, Igepal DM-430®, and Coriander oil.

Another embodiment comprises cyclosporin A, Igepal DM-530®, and Coriander oil.

Another embodiment comprises cyclosporin A, Igepal DM-710®, and Coriander oil.

Another embodiment comprises cyclosporin A, Igepal DM-730®, and Coriander oil.

Another embodiment comprises cyclosporin A, Igepal DM-880®, and Coriander oil.

Another embodiment comprises cyclosporin A, Igepal DM-970 FLK®, and Coriander oil.

Another embodiment comprises cyclosporin A, Igepal OD-410®, and Coriander oil.

Another embodiment comprises cyclosporin A, Igepal RC-520®, and Coriander oil.

Another embodiment comprises cyclosporin A, Igepal RC-620®, and Coriander oil.

Another embodiment comprises cyclosporin A, Igepal RC-630®, and Coriander oil.

Another embodiment comprises cyclosporin A, Lexemul CS-20®, and Coriander oil.

Another embodiment comprises cyclosporin A, Lipo GMS 450®, and Coriander oil.

Another embodiment comprises cyclosporin A, Lipo Polyglycol 1000®, and Coriander oil.

Another embodiment comprises cyclosporin A, Lipo Polyglycol 200®, and Coriander oil.

Another embodiment comprises cyclosporin A, Lipo Polyglycol 300®, and Coriander oil.

Another embodiment comprises cyclosporin A, Lipo Polyglycol 3350®, and Coriander oil.

Another embodiment comprises cyclosporin A, Lipo Polyglycol 400®, and Coriander oil.

Another embodiment comprises cyclosporin A, Lipo Polyglycol 600®, and Coriander oil.

Another embodiment comprises cyclosporin A, Lipocol C-10®, and Coriander oil.

Another embodiment comprises cyclosporin A, Lipocol C-2®, and Coriander oil.

Another embodiment comprises cyclosporin A, Lipocol C-20®, and Coriander oil.

Another embodiment comprises cyclosporin A, Lipocol L-12®, and Coriander oil.

Another embodiment comprises cyclosporin A, Lipocol L-23®, and Coriander oil.

Another embodiment comprises cyclosporin A, Lipocol L-4®, and Coriander oil.

Another embodiment comprises cyclosporin A, Lipocol O-10®, and Coriander oil.

Another embodiment comprises cyclosporin A, Lipocol O-2®, and Coriander oil.

Another embodiment comprises cyclosporin A, Lipocol O-20®, and Coriander oil.

Another embodiment comprises cyclosporin A, Lipocol O-5®, and Coriander oil.

Another embodiment comprises cyclosporin A, Lipocol S-10®, and Coriander oil.

Another embodiment comprises cyclosporin A, Lipocol S-2®, and Coriander oil.

Another embodiment comprises cyclosporin A, Lipocol S-20®, and Coriander oil.

Another embodiment comprises cyclosporin A, Lipocol SC-15®, and Coriander oil.

Another embodiment comprises cyclosporin A, Lipocol SC-20®, and Coriander oil.

Another embodiment comprises cyclosporin A, Lipocol SC-4®, and Coriander oil.

Another embodiment comprises cyclosporin A, Lipocol TD-12®, and Coriander oil.

Another embodiment comprises cyclosporin A, Lumulse 40-O K®, and Coriander oil.

Another embodiment comprises cyclosporin A, Lumulse 42-O K®, and Coriander oil.

Another embodiment comprises cyclosporin A, Lumulse GMT-40®, and Coriander oil.

Another embodiment comprises cyclosporin A, Lumulse PEG 1450®, and Coriander oil.

Another embodiment comprises cyclosporin A, Lumulse PEG 1450 NF®, and Coriander oil.

Another embodiment comprises cyclosporin A, Lumulse PEG 8000®, and Coriander oil.

Another embodiment comprises cyclosporin A, Lumulse PGO®, and Coriander oil.

Another embodiment comprises cyclosporin A, Lumulse POE (2) Oleyl Amine®, and Coriander oil.

Another embodiment comprises cyclosporin A, Magrabar PGO-104®, and Coriander oil.

Another embodiment comprises cyclosporin A, Mapeg 200 ML®, and Coriander oil.

Another embodiment comprises cyclosporin A, Mapeg 400 MO®, and Coriander oil.

Another embodiment comprises cyclosporin A, Mapeg 400 MOT®, and Coriander oil.

Another embodiment comprises cyclosporin A, Mapeg 400DO®, and Coriander oil.

Another embodiment comprises cyclosporin A, Mapeg 400DOT®, and Coriander oil.

Another embodiment comprises cyclosporin A, Mapeg 600DOT®, and Coriander oil.

Another embodiment comprises cyclosporin A, Marlox B 24/80®, and Coriander oil.

Another embodiment comprises cyclosporin A, Marlox FK 86®, and Coriander oil.

Another embodiment comprises cyclosporin A, Marlox MO 154®, and Coriander oil.

Another embodiment comprises cyclosporin A, Mazox CDA CG®, and Coriander oil.

Another embodiment comprises cyclosporin A, Monoglycerides Glyceryl Monestearate®, and Coriander oil.

Another embodiment comprises cyclosporin A, Myrj 45®, and Coriander oil.

Another embodiment comprises cyclosporin A, Myrj 52®, and Coriander oil.

Another embodiment comprises cyclosporin A, Myrj 52S®, and Coriander oil.

Another embodiment comprises cyclosporin A, Myrj 53®, and Coriander oil.

Another embodiment comprises cyclosporin A, Myrj 59®, and Coriander oil.

Another embodiment comprises cyclosporin A, Norfox NP-12®, and Coriander oil.

Another embodiment comprises cyclosporin A, Norfox NP-4®, and Coriander oil.

Another embodiment comprises cyclosporin A, Norfox NP-6®, and Coriander oil.

Another embodiment comprises cyclosporin A, Norfox NP-887®, and Coriander oil.

Another embodiment comprises cyclosporin A, Norfox NP-9®, and Coriander oil.

Another embodiment comprises cyclosporin A, Norfox NP-977®, and Coriander oil.

Another embodiment comprises cyclosporin A, Norfox OP-100®, and Coriander oil.

Another embodiment comprises cyclosporin A, Norfox OP-102®, and Coriander oil.

Another embodiment comprises cyclosporin A, Norfox OP-114®, and Coriander oil.

Another embodiment comprises cyclosporin A, Norfox OP-45®, and Coriander oil.

Another embodiment comprises cyclosporin A, Pegosperse 100-L®, and Coriander oil.

Another embodiment comprises cyclosporin A, Pegosperse 100-O®, and Coriander oil.

Another embodiment comprises cyclosporin A, Pegosperse 100-S®, and Coriander oil.

Another embodiment comprises cyclosporin A, Pegosperse 1500-MS®, and Coriander oil.

Another embodiment comprises cyclosporin A, Pegosperse 1750-MS®, and Coriander oil.

Another embodiment comprises cyclosporin A, Pegosperse 200 DL®, and Coriander oil.

Another embodiment comprises cyclosporin A, Pegosperse 200-ML®, and Coriander oil.

Another embodiment comprises cyclosporin A, Pegosperse 400-DL®, and Coriander oil.

Another embodiment comprises cyclosporin A, Pegosperse 400-DO®, and Coriander oil.

Another embodiment comprises cyclosporin A, Pegosperse 400-DS®, and Coriander oil.

Another embodiment comprises cyclosporin A, Pegosperse 400-ML®, and Coriander oil.

Another embodiment comprises cyclosporin A, Pegosperse 400-MO®, and Coriander oil.

Another embodiment comprises cyclosporin A, Pegosperse 400-MOT®, and Coriander oil.

Another embodiment comprises cyclosporin A, Pegosperse 400-MS®, and Coriander oil.

Another embodiment comprises cyclosporin A, Pegosperse 50-DS®, and Coriander oil.

Another embodiment comprises cyclosporin A, Pegosperse 50-MS®, and Coriander oil.

Another embodiment comprises cyclosporin A, Pegosperse 600-ML®, and Coriander oil.

Another embodiment comprises cyclosporin A, Pegosperse 600-MS®, and Coriander oil.

Another embodiment comprises cyclosporin A, Plurafac A-38®, and Coriander oil.

Another embodiment comprises cyclosporin A, Plurafac B-25-5®, and Coriander oil.

Another embodiment comprises cyclosporin A, Plurafac B-26®, and Coriander oil.

Another embodiment comprises cyclosporin A, Plurafac D-25®, and Coriander oil.

Another embodiment comprises cyclosporin A, Plurafac LF 131®, and Coriander oil.

Another embodiment comprises cyclosporin A, Plurafac LF 4030®, and Coriander oil.

Another embodiment comprises cyclosporin A, Plurafac LF 7000®, and Coriander oil.

Another embodiment comprises cyclosporin A, Plurafac RA-20®, and Coriander oil.

Another embodiment comprises cyclosporin A, Plurafac RA-30®, and Coriander oil.

Another embodiment comprises cyclosporin A, Plurafac RA-40®, and Coriander oil.

Another embodiment comprises cyclosporin A, Pluronic 10R5®, and Coriander oil.

Another embodiment comprises cyclosporin A, Pluronic 17R2®, and Coriander oil.

Another embodiment comprises cyclosporin A, Pluronic 17R4®, and Coriander oil.

Another embodiment comprises cyclosporin A, Pluronic 25R2®, and Coriander oil.

Another embodiment comprises cyclosporin A, Pluronic 25R4®, and Coriander oil.

Another embodiment comprises cyclosporin A, Pluronic 31R1®, and Coriander oil.

Another embodiment comprises cyclosporin A, Pluronic F108®, and Coriander oil.

Another embodiment comprises cyclosporin A, Pluronic F127®, and Coriander oil.

Another embodiment comprises cyclosporin A, Pluronic F38®, and Coriander oil.

Another embodiment comprises cyclosporin A, Pluronic F68®, and Coriander oil.

Another embodiment comprises cyclosporin A, Pluronic F68LF®, and Coriander oil.

Another embodiment comprises cyclosporin A, Pluronic F77®, and Coriander oil.

Another embodiment comprises cyclosporin A, Pluronic F87®, and Coriander oil.

Another embodiment comprises cyclosporin A, Pluronic F88®, and Coriander oil.

Another embodiment comprises cyclosporin A, Pluronic F98®, and Coriander oil.

Another embodiment comprises cyclosporin A, Pluronic L10®, and Coriander oil.

Another embodiment comprises cyclosporin A, Pluronic L101®, and Coriander oil.

Another embodiment comprises cyclosporin A, Pluronic L121®, and Coriander oil.

Another embodiment comprises cyclosporin A, Pluronic L31®, and Coriander oil.

Another embodiment comprises cyclosporin A, Pluronic L35®, and Coriander oil.

Another embodiment comprises cyclosporin A, Pluronic L43®, and Coriander oil.

Another embodiment comprises cyclosporin A, Pluronic L44®, and Coriander oil.

Another embodiment comprises cyclosporin A, Pluronic L61®, and Coriander oil.

Another embodiment comprises cyclosporin A, Pluronic L62®, and Coriander oil.

Another embodiment comprises cyclosporin A, Pluronic L62D®, and Coriander oil.

Another embodiment comprises cyclosporin A, Pluronic L62LF®, and Coriander oil.

Another embodiment comprises cyclosporin A, Pluronic L64®, and Coriander oil.

Another embodiment comprises cyclosporin A, Pluronic L81®, and Coriander oil.

Another embodiment comprises cyclosporin A, Pluronic L92®, and Coriander oil.

Another embodiment comprises cyclosporin A, Pluronic P103®, and Coriander oil.

Another embodiment comprises cyclosporin A, Pluronic P104®, and Coriander oil.

Another embodiment comprises cyclosporin A, Pluronic P105®, and Coriander oil.

Another embodiment comprises cyclosporin A, Pluronic P123®, and Coriander oil.

Another embodiment comprises cyclosporin A, Pluronic P65®, and Coriander oil.

Another embodiment comprises cyclosporin A, Pluronic P84®, and Coriander oil.

Another embodiment comprises cyclosporin A, Pluronic P85®, and Coriander oil.

Another embodiment comprises cyclosporin A, Polyaldo 10-2-P®, and Coriander oil.

Another embodiment comprises cyclosporin A, Polyaldo DGDO®, and Coriander oil.

Another embodiment comprises cyclosporin A, Polyaldo HGDS®, and Coriander oil.

Another embodiment comprises cyclosporin A, Polyaldo TGMS®, and Coriander oil.

Another embodiment comprises cyclosporin A, Protegin V®, and Coriander oil.

Another embodiment comprises cyclosporin A, Protegin W®, and Coriander oil.

Another embodiment comprises cyclosporin A, Protegin WX®, and Coriander oil.

Another embodiment comprises cyclosporin A, Protegin XV®, and Coriander oil.

Another embodiment comprises cyclosporin A, Rhodasurf BC-420®, and Coriander oil.

Another embodiment comprises cyclosporin A, Rhodasurf BC-610®, and Coriander oil.

Another embodiment comprises cyclosporin A, Rhodasurf BC-630®, and Coriander oil.

Another embodiment comprises cyclosporin A, Rhodasurf BC-720®, and Coriander oil.

Another embodiment comprises cyclosporin A, Rhodasurf BC-840®, and Coriander oil.

Another embodiment comprises cyclosporin A, Rhodasurf L 1®, and Coriander oil.

Another embodiment comprises cyclosporin A, Rhodasurf L 12®, and Coriander oil.

Another embodiment comprises cyclosporin A, Rhodasurf L 2®, and Coriander oil.

Another embodiment comprises cyclosporin A, Rhodasurf L 20®, and Coriander oil.

Another embodiment comprises cyclosporin A, Rhodasurf L 3®, and Coriander oil.

Another embodiment comprises cyclosporin A, Rhodasurf L 30®, and Coriander oil.

Another embodiment comprises cyclosporin A, Rhodasurf L 4®, and Coriander oil.

Another embodiment comprises cyclosporin A, Rhodasurf L 50®, and Coriander oil.

Another embodiment comprises cyclosporin A, Rhodasurf L 7/90®, and Coriander oil.

Another embodiment comprises cyclosporin A, Rhodasurf LA-3®, and Coriander oil.

Another embodiment comprises cyclosporin A, Rhodasurf LA-7®, and Coriander oil.

Another embodiment comprises cyclosporin A, Rhodasurf LAN-23®, and Coriander oil.

Another embodiment comprises cyclosporin A, Rhodasurf ON-870®, and Coriander oil.

Another embodiment comprises cyclosporin A, Rhodasurf ON-877®, and Coriander oil.

Another embodiment comprises cyclosporin A, Rhodasurf TDA-8/5®, and Coriander oil.

Another embodiment comprises cyclosporin A, Ritapro 100®, and Coriander oil.

Another embodiment comprises cyclosporin A, Ritapro 165®, and Coriander oil.

Another embodiment comprises cyclosporin A, Ritapro 200®, and Coriander oil.

Another embodiment comprises cyclosporin A, Ritapro 300®, and Coriander oil.

Another embodiment comprises cyclosporin A, Ritoleth 10®, and Coriander oil.

Another embodiment comprises cyclosporin A, Ritoleth 2®, and Coriander oil.

Another embodiment comprises cyclosporin A, Ritoleth 20®, and Coriander oil.

Another embodiment comprises cyclosporin A, Ritoleth 5®, and Coriander oil.

Another embodiment comprises cyclosporin A, Santone 10-10-0®, and Coriander oil.

Another embodiment comprises cyclosporin A, Santone 3-1-S XTR®, and Coriander oil.

Another embodiment comprises cyclosporin A, Santone 3-1-SH®, and Coriander oil.

Another embodiment comprises cyclosporin A, Santone 8-1-O®, and Coriander oil.

Another embodiment comprises cyclosporin A, Solulan 16®, and Coriander oil.

Another embodiment comprises cyclosporin A, Solulan 75®, and Coriander oil.

Another embodiment comprises cyclosporin A, Solulan C-24®, and Coriander oil.

Another embodiment comprises cyclosporin A, Solulan L-575®, and Coriander oil.

Another embodiment comprises cyclosporin A, Standamox CAW®, and Coriander oil.

Another embodiment comprises cyclosporin A, Surfine AZI-A®, and Coriander oil.

Another embodiment comprises cyclosporin A, Surfonic CO-15®, and Coriander oil.

Another embodiment comprises cyclosporin A, Surfonic CO-25®, and Coriander oil.

Another embodiment comprises cyclosporin A, Surfonic CO-30®, and Coriander oil.

Another embodiment comprises cyclosporin A, Surfonic CO-36®, and Coriander oil.

Another embodiment comprises cyclosporin A, Surfonic CO-42®, and Coriander oil.

Another embodiment comprises cyclosporin A, Surfonic E400-MO®, and Coriander oil.

Another embodiment comprises cyclosporin A, Surfonic L12-3®, and Coriander oil.

Another embodiment comprises cyclosporin A, Surfonic L12-6®, and Coriander oil.

Another embodiment comprises cyclosporin A, Surfonic L12-8®, and Coriander oil.

Another embodiment comprises cyclosporin A, Surfonic L24-12®, and Coriander oil.

Another embodiment comprises cyclosporin A, Surfonic L24-2®, and Coriander oil.

Another embodiment comprises cyclosporin A, Surfonic L24-22®, and Coriander oil.

Another embodiment comprises cyclosporin A, Surfonic L24-3®, and Coriander oil.

Another embodiment comprises cyclosporin A, Surfonic L24-4®, and Coriander oil.

Another embodiment comprises cyclosporin A, Surfonic L24-5®, and Coriander oil.

Another embodiment comprises cyclosporin A, Surfonic L24-7®, and Coriander oil.

Another embodiment comprises cyclosporin A, Surfonic L24-7.1®, and Coriander oil.

Another embodiment comprises cyclosporin A, Surfonic L24-9®, and Coriander oil.

Another embodiment comprises cyclosporin A, Surfonic LF-17®, and Coriander oil.

Another embodiment comprises cyclosporin A, Surfonic LF-18®, and Coriander oil.

Another embodiment comprises cyclosporin A, Surfonic LF-37®, and Coriander oil.

Another embodiment comprises cyclosporin A, Surfonic LF-41®, and Coriander oil.

Another embodiment comprises cyclosporin A, Surfonic LF-42®, and Coriander oil.

Another embodiment comprises cyclosporin A, Surfonic LF-47®, and Coriander oil.

Another embodiment comprises cyclosporin A, Surfonic LF-68®, and Coriander oil.

Another embodiment comprises cyclosporin A, Surfonic N-10®, and Coriander oil.

Another embodiment comprises cyclosporin A, Surfonic N-100®, and Coriander oil.

Another embodiment comprises cyclosporin A, Surfonic N-102®, and Coriander oil.

Another embodiment comprises cyclosporin A, Surfonic N-120®, and Coriander oil.

Another embodiment comprises cyclosporin A, Surfonic N-150®, and Coriander oil.

Another embodiment comprises cyclosporin A, Surfonic N-200®, and Coriander oil.

Another embodiment comprises cyclosporin A, Surfonic N-300®, and Coriander oil.

Another embodiment comprises cyclosporin A, Surfonic N-31.5®, and Coriander oil.

Another embodiment comprises cyclosporin A, Surfonic N-40®, and Coriander oil.

Another embodiment comprises cyclosporin A, Surfonic N-400®, and Coriander oil.

Another embodiment comprises cyclosporin A, Surfonic N-60®, and Coriander oil.

Another embodiment comprises cyclosporin A, Surfonic N-85®, and Coriander oil.

Another embodiment comprises cyclosporin A, Surfonic N-95®, and Coriander oil.

Another embodiment comprises cyclosporin A, Surfonic NB-18®, and Coriander oil.

Another embodiment comprises cyclosporin A, Surfonic NB-23®, and Coriander oil.

Another embodiment comprises cyclosporin A, Surfonic NB-25®, and Coriander oil.

Another embodiment comprises cyclosporin A, Surfonic NB-307®, and Coriander oil.

Another embodiment comprises cyclosporin A, Surfonic NB-31®, and Coriander oil.

Another embodiment comprises cyclosporin A, Surfonic NB-41®, and Coriander oil.

Another embodiment comprises cyclosporin A, Surfonic NB-43®, and Coriander oil.

Another embodiment comprises cyclosporin A, Surfonic OP-100®, and Coriander oil.

Another embodiment comprises cyclosporin A, Surfonic OP-120®, and Coriander oil.

Another embodiment comprises cyclosporin A, Surfonic OP-15®, and Coriander oil.

Another embodiment comprises cyclosporin A, Surfonic OP-35®, and Coriander oil.

Another embodiment comprises cyclosporin A, Surfonic OP-70®, and Coriander oil.

Another embodiment comprises cyclosporin A, Surfonic P1®, and Coriander oil.

Another embodiment comprises cyclosporin A, Surfonic P3®, and Coriander oil.

Another embodiment comprises cyclosporin A, Surfonic P5®, and Coriander oil.

Another embodiment comprises cyclosporin A, Surfonic P6®, and Coriander oil.

Another embodiment comprises cyclosporin A, Surfonic POA-17R2®, and Coriander oil.

Another embodiment comprises cyclosporin A, Surfonic POA-25R2®, and Coriander oil.

Another embodiment comprises cyclosporin A, Surfonic TDA-11®, and Coriander oil.

Another embodiment comprises cyclosporin A, Surfonic TDA-3B®, and Coriander oil.

Another embodiment comprises cyclosporin A, Surfonic TDA-6®, and Coriander oil.

Another embodiment comprises cyclosporin A, Surfonic TDA-6/88®, and Coriander oil.

Another embodiment comprises cyclosporin A, Surfonic TDA-8®, and Coriander oil.

Another embodiment comprises cyclosporin A, Surfonic TDA-8/90®, and Coriander oil.

Another embodiment comprises cyclosporin A, Surfonic TDA-9®, and Coriander oil.

Another embodiment comprises cyclosporin A, Surfonic TX-CCN®, and Coriander oil.

Another embodiment comprises cyclosporin A, Tagat CH 40®, and Coriander oil.

Another embodiment comprises cyclosporin A, Tagat CH 60®, and Coriander oil.

Another embodiment comprises cyclosporin A, Tagat L 2®, and Coriander oil.

Another embodiment comprises cyclosporin A, Tagat S®, and Coriander oil.

Another embodiment comprises cyclosporin A, Tagat S 2®, and Coriander oil.

Another embodiment comprises cyclosporin A, Tally 100 Plus®, and Coriander oil.

Another embodiment comprises cyclosporin A, T-Det A106®, and Coriander oil.

Another embodiment comprises cyclosporin A, T-Det A109®, and Coriander oil.

Another embodiment comprises cyclosporin A, T-Det A136®, and Coriander oil.

Another embodiment comprises cyclosporin A, T-Det A139®, and Coriander oil.

Another embodiment comprises cyclosporin A, T-Det A2412®, and Coriander oil.

Another embodiment comprises cyclosporin A, T-Det A243®, and Coriander oil.

Another embodiment comprises cyclosporin A, T-Det A247®, and Coriander oil.

Another embodiment comprises cyclosporin A, T-Det A249®, and Coriander oil.

Another embodiment comprises cyclosporin A, T-Det A467®, and Coriander oil.

Another embodiment comprises cyclosporin A, T-Det COE®, and Coriander oil.

Another embodiment comprises cyclosporin A, T-Det DD-10®, and Coriander oil.

Another embodiment comprises cyclosporin A, T-Det DD-5®, and Coriander oil.

Another embodiment comprises cyclosporin A, T-Det DD-7®, and Coriander oil.

Another embodiment comprises cyclosporin A, T-Det EPO-104®, and Coriander oil.

Another embodiment comprises cyclosporin A, T-Det EPO-61®, and Coriander oil.

Another embodiment comprises cyclosporin A, T-Det EPO-62®, and Coriander oil.

Another embodiment comprises cyclosporin A, T-Det EPO-64®, and Coriander oil.

Another embodiment comprises cyclosporin A, T-Det N-1.5®, and Coriander oil.

Another embodiment comprises cyclosporin A, T-Det N-10.5®, and Coriander oil.

Another embodiment comprises cyclosporin A, T-Det N-100®, and Coriander oil.

Another embodiment comprises cyclosporin A, T-Det N-1007®, and Coriander oil.

Another embodiment comprises cyclosporin A, T-Det N-12®, and Coriander oil.

Another embodiment comprises cyclosporin A, T-Det N-14®, and Coriander oil.

Another embodiment comprises cyclosporin A, T-Det N-20®, and Coriander oil.

Another embodiment comprises cyclosporin A, T-Det N-30®, and Coriander oil.

Another embodiment comprises cyclosporin A, T-Det N-307®, and Coriander oil.

Another embodiment comprises cyclosporin A, T-Det N-4®, and Coriander oil.

Another embodiment comprises cyclosporin A, T-Det N-40®, and Coriander oil.

Another embodiment comprises cyclosporin A, T-Det N-407®, and Coriander oil.

Another embodiment comprises cyclosporin A, T-Det N-50®, and Coriander oil.

Another embodiment comprises cyclosporin A, T-Det N-507®, and Coriander oil.

Another embodiment comprises cyclosporin A, T-Det N-6®, and Coriander oil.

Another embodiment comprises cyclosporin A, T-Det N-70®, and Coriander oil.

Another embodiment comprises cyclosporin A, T-Det N-8®, and Coriander oil.

Another embodiment comprises cyclosporin A, T-Det N-9.5®, and Coriander oil.

Another embodiment comprises cyclosporin A, T-Det O-12®, and Coriander oil.

Another embodiment comprises cyclosporin A, T-Det O-165®, and Coriander oil.

Another embodiment comprises cyclosporin A, T-Det O-307®, and Coriander oil.

Another embodiment comprises cyclosporin A, T-Det O-40®, and Coriander oil.

Another embodiment comprises cyclosporin A, T-Det O-407®, and Coriander oil.

Another embodiment comprises cyclosporin A, T-Det O-6®, and Coriander oil.

Another embodiment comprises cyclosporin A, T-Det O-8®, and Coriander oil.

Another embodiment comprises cyclosporin A, T-Det O-9®, and Coriander oil.

Another embodiment comprises cyclosporin A, Tegin®, and Coriander oil.

Another embodiment comprises cyclosporin A, Tergitol 15-S-12®, and Coriander oil.

Another embodiment comprises cyclosporin A, Tergitol 15-S-15®, and Coriander oil.

Another embodiment comprises cyclosporin A, Tergitol 15-S-20®, and Coriander oil.

Another embodiment comprises cyclosporin A, Tergitol 15-S-20 (80AQ)®, and Coriander oil.

Another embodiment comprises cyclosporin A, Tergitol 15-S-3®, and Coriander oil.

Another embodiment comprises cyclosporin A, Tergitol 15-S-30®, and Coriander oil.

Another embodiment comprises cyclosporin A, Tergitol 15-S-40®, and Coriander oil.

Another embodiment comprises cyclosporin A, Tergitol 15-S-40 (70AQ)®, and Coriander oil.

Another embodiment comprises cyclosporin A, Tergitol 15-S-5®, and Coriander oil.

Another embodiment comprises cyclosporin A, Tergitol 15-S-7®, and Coriander oil.

Another embodiment comprises cyclosporin A, Tergitol 15-S-9®, and Coriander oil.

Another embodiment comprises cyclosporin A, Tergitol L-101®, and Coriander oil.

Another embodiment comprises cyclosporin A, Tergitol L-61®, and Coriander oil.

Another embodiment comprises cyclosporin A, Tergitol L-62®, and Coriander oil.

Another embodiment comprises cyclosporin A, Tergitol L-64®, and Coriander oil.

Another embodiment comprises cyclosporin A, Tergitol L-81®, and Coriander oil.

Another embodiment comprises cyclosporin A, Tergitol NP-10®, and Coriander oil.

Another embodiment comprises cyclosporin A, Tergitol NP-11®, and Coriander oil.

Another embodiment comprises cyclosporin A, Tergitol NP-12®, and Coriander oil.

Another embodiment comprises cyclosporin A, Tergitol NP-13®, and Coriander oil.

Another embodiment comprises cyclosporin A, Tergitol NP-15®, and Coriander oil.

Another embodiment comprises cyclosporin A, Tergitol NP-30®, and Coriander oil.

Another embodiment comprises cyclosporin A, Tergitol NP-30 (70 AQ)®, and Coriander oil.

Another embodiment comprises cyclosporin A, Tergitol NP-4®, and Coriander oil.

Another embodiment comprises cyclosporin A, Tergitol NP-40®, and Coriander oil.

Another embodiment comprises cyclosporin A, Tergitol NP-40 (70 AQ)®, and Coriander oil.

Another embodiment comprises cyclosporin A, Tergitol NP-50 (70 AQ)®, and Coriander oil.

Another embodiment comprises cyclosporin A, Tergitol NP-55 (70 AQ)®, and Coriander oil.

Another embodiment comprises cyclosporin A, Tergitol NP-6®, and Coriander oil.

Another embodiment comprises cyclosporin A, Tergitol NP-7®, and Coriander oil.

Another embodiment comprises cyclosporin A, Tergitol NP-70 (70 AQ)®, and Coriander oil.

Another embodiment comprises cyclosporin A, Tergitol NP-8®, and Coriander oil.

Another embodiment comprises cyclosporin A, Tergitol NP-9®, and Coriander oil.

Another embodiment comprises cyclosporin A, Tergitol NP-9.5®, and Coriander oil.

Another embodiment comprises cyclosporin A, Tergitol TMN-10®, and Coriander oil.

Another embodiment comprises cyclosporin A, Tergitol TMN-3®, and Coriander oil.

Another embodiment comprises cyclosporin A, Tergitol TMN-6®, and Coriander oil.

Another embodiment comprises cyclosporin A, Tergitol XD®, and Coriander oil.

Another embodiment comprises cyclosporin A, Tergitol XH®, and Coriander oil.

Another embodiment comprises cyclosporin A, Tergitol XJ®, and Coriander oil.

Another embodiment comprises cyclosporin A, Tetronic 1107®, and Coriander oil.

Another embodiment comprises cyclosporin A, Tetronic 1307®, and Coriander oil.

Another embodiment comprises cyclosporin A, Tetronic 150R1®, and Coriander oil.

Another embodiment comprises cyclosporin A, Tetronic 304®, and Coriander oil.

Another embodiment comprises cyclosporin A, Tetronic 701®, and Coriander oil.

Another embodiment comprises cyclosporin A, Tetronic 901®, and Coriander oil.

Another embodiment comprises cyclosporin A, Tetronic 904®, and Coriander oil.

Another embodiment comprises cyclosporin A, Tetronic 908®, and Coriander oil.

Another embodiment comprises cyclosporin A, Tetronic 90R4®, and Coriander oil.

Another embodiment comprises cyclosporin A, Tomadol 1-3®, and Coriander oil.

Another embodiment comprises cyclosporin A, Tomadol 1-5®, and Coriander oil.

Another embodiment comprises cyclosporin A, Tomadol 1-7®, and Coriander oil.

Another embodiment comprises cyclosporin A, Tomadol 23-1®, and Coriander oil.

Another embodiment comprises cyclosporin A, Tomadol 23-3®, and Coriander oil.

Another embodiment comprises cyclosporin A, Tomadol 23-5®, and Coriander oil.

Another embodiment comprises cyclosporin A, Tomadol 23-6.5®, and Coriander oil.

Another embodiment comprises cyclosporin A, Tomadol 25-12®, and Coriander oil.

Another embodiment comprises cyclosporin A, Tomadol 25-3®, and Coriander oil.

Another embodiment comprises cyclosporin A, Tomadol 25-7®, and Coriander oil.

Another embodiment comprises cyclosporin A, Tomadol 25-9®, and Coriander oil.

Another embodiment comprises cyclosporin A, Tomadol 45-13®, and Coriander oil.

Another embodiment comprises cyclosporin A, Tomadol 45-2.5®, and Coriander oil.

Another embodiment comprises cyclosporin A, Tomadol 45-7®, and Coriander oil.

Another embodiment comprises cyclosporin A, Tomadol 91-2.5®, and Coriander oil.

Another embodiment comprises cyclosporin A, Tomadol 91-6®, and Coriander oil.

Another embodiment comprises cyclosporin A, Tomadol 91-8®, and Coriander oil.

Another embodiment comprises cyclosporin A, Triton X-100®, and Coriander oil.

Another embodiment comprises cyclosporin A, Triton X-102®, and Coriander oil.

Another embodiment comprises cyclosporin A, Triton X-114®, and Coriander oil.

Another embodiment comprises cyclosporin A, Triton X-15®, and Coriander oil.

Another embodiment comprises cyclosporin A, Triton X-165 (70%)0®, and Coriander oil.

Another embodiment comprises cyclosporin A, Triton X-207®, and Coriander oil.

Another embodiment comprises cyclosporin A, Triton X-305 (70%)®, and Coriander oil.

Another embodiment comprises cyclosporin A, Triton X-35®, and Coriander oil.

Another embodiment comprises cyclosporin A, Triton X-405 (70%)®, and Coriander oil.

Another embodiment comprises cyclosporin A, Triton X-45®, and Coriander oil.

Another embodiment comprises cyclosporin A, Triton X-705 (70%)®, and Coriander oil.

Another embodiment comprises cyclosporin A, Volpo S-10®, and Coriander oil.

Another embodiment comprises cyclosporin A, Volpo S-2®, and Coriander oil.

Another embodiment comprises cyclosporin A, Volpo S-20®, and Coriander oil.

Another embodiment comprises cyclosporin A, Volpo-10®, and Coriander oil.

Another embodiment comprises cyclosporin A, Volpo-20®, and Coriander oil.

Another embodiment comprises cyclosporin A, Volpo-3®, and Coriander oil.

Another embodiment comprises cyclosporin A, Volpo-5®, and Coriander oil.

Another embodiment comprises cyclosporin A, Abex 2515®, and Lavender oil.

Another embodiment comprises cyclosporin A, Abex 2525/40®, and Lavender oil.

Another embodiment comprises cyclosporin A, Abex 2535®, and Lavender oil.

Another embodiment comprises cyclosporin A, Abex 2545®, and Lavender oil.

Another embodiment comprises cyclosporin A, Acconon E®, and Lavender oil.

Another embodiment comprises cyclosporin A, Alfonic 1216CO-9®, and Lavender oil.

Another embodiment comprises cyclosporin A, Alfonic 1412-3®, and Lavender oil.

Another embodiment comprises cyclosporin A, Alfonic TDA-12®, and Lavender oil.

Another embodiment comprises cyclosporin A, Alfonic TDA-3®, and Lavender oil.

Another embodiment comprises cyclosporin A, Alfonic TDA-4®, and Lavender oil.

Another embodiment comprises cyclosporin A, Alfonic TDA-6®, and Lavender oil.

Another embodiment comprises cyclosporin A, Alfonic TDA-7®, and Lavender oil.

Another embodiment comprises cyclosporin A, Alfonic TDA-8®, and Lavender oil.

Another embodiment comprises cyclosporin A, Alfonic TDA-9®, and Lavender oil.

Another embodiment comprises cyclosporin A, Alkamuls 400-DO®, and Lavender oil.

Another embodiment comprises cyclosporin A, Alkamuls 400-MO/E®, and Lavender oil.

Another embodiment comprises cyclosporin A, Ameroxol OE-10®, and Lavender oil.

Another embodiment comprises cyclosporin A, Ameroxol OE-2®, and Lavender oil.

Another embodiment comprises cyclosporin A, Ameroxol OE-20®, and Lavender oil.

Another embodiment comprises cyclosporin A, Armix 176®, and Lavender oil.

Another embodiment comprises cyclosporin A, Armix 180-C®, and Lavender oil.

Another embodiment comprises cyclosporin A, Armix 183®, and Lavender oil.

Another embodiment comprises cyclosporin A, Brij 35®, and Lavender oil.

Another embodiment comprises cyclosporin A, Brij 35 Liquids, and Lavender oil.

Another embodiment comprises cyclosporin A, Brij 35 SP®, and Lavender oil.

Another embodiment comprises cyclosporin A, Canasol MJ 2109®, and Lavender oil.

Another embodiment comprises cyclosporin A, Canasol MJ 52®, and Lavender oil.

Another embodiment comprises cyclosporin A, Canasol R 3600®, and Lavender oil.

Another embodiment comprises cyclosporin A, Canasol R 3603®, and Lavender oil.

Another embodiment comprises cyclosporin A, Canasol R 4000 H®, and Lavender oil.

Another embodiment comprises cyclosporin A, Cedepal CA-210®, and Lavender oil.

Another embodiment comprises cyclosporin A, Cedepal CA-520®, and Lavender oil.

Another embodiment comprises cyclosporin A, Cedepal CA-630®, and Lavender oil.

Another embodiment comprises cyclosporin A, Cedepal CA-720®, and Lavender oil.

Another embodiment comprises cyclosporin A, Cedepal CA-890®, and Lavender oil.

Another embodiment comprises cyclosporin A, Cedepal CA-897®, and Lavender oil.

Another embodiment comprises cyclosporin A, Cedepal CO-210®, and Lavender oil.

Another embodiment comprises cyclosporin A, Cedepal CO-430®, and Lavender oil.

Another embodiment comprises cyclosporin A, Cedepal CO-530®, and Lavender oil.

Another embodiment comprises cyclosporin A, Cedepal CO-630®, and Lavender oil.

Another embodiment comprises cyclosporin A, Cedepal CO-710®, and Lavender oil.

Another embodiment comprises cyclosporin A, Cedepal CO-730®, and Lavender oil.

Another embodiment comprises cyclosporin A, Cedepal CO-880®, and Lavender oil.

Another embodiment comprises cyclosporin A, Cedepal CO-887®, and Lavender oil.

Another embodiment comprises cyclosporin A, Cedepal CO-890®, and Lavender oil.

Another embodiment comprises cyclosporin A, Cedepal CO-897®, and Lavender oil.

Another embodiment comprises cyclosporin A, Cedepal CO-970®, and Lavender oil.

Another embodiment comprises cyclosporin A, Cedepal CO-977®, and Lavender oil.

Another embodiment comprises cyclosporin A, Cedepal CO-990®, and Lavender oil.

Another embodiment comprises cyclosporin A, Cedepal CO-997®, and Lavender oil.

Another embodiment comprises cyclosporin A, Chemal BP 261®, and Lavender oil.

Another embodiment comprises cyclosporin A, Chemal BP 262®, and Lavender oil.

Another embodiment comprises cyclosporin A, Chemal BP 3172®, and Lavender oil.

Another embodiment comprises cyclosporin A, Chemal BP 3174®, and Lavender oil.

Another embodiment comprises cyclosporin A, Chemal DA-4®, and Lavender oil.

Another embodiment comprises cyclosporin A, Chemal DA-6®, and Lavender oil.

Another embodiment comprises cyclosporin A, Chemal DA-9®, and Lavender oil.

Another embodiment comprises cyclosporin A, Chemal LA-23®, and Lavender oil.

Another embodiment comprises cyclosporin A, Chemal LA-4®, and Lavender oil.

Another embodiment comprises cyclosporin A, Chemal LA-9®, and Lavender oil.

Another embodiment comprises cyclosporin A, Chemal OA-20G®, and Lavender oil.

Another embodiment comprises cyclosporin A, Chemal OA-5®, and Lavender oil.

Another embodiment comprises cyclosporin A, Chemal OA-9®, and Lavender oil.

Another embodiment comprises cyclosporin A, Chemal TDA-12®, and Lavender oil.

Another embodiment comprises cyclosporin A, Chemal TDA-15®, and Lavender oil.

Another embodiment comprises cyclosporin A, Chemal TDA-3®, and Lavender oil.

Another embodiment comprises cyclosporin A, Chemal TDA-6®, and Lavender oil.

Another embodiment comprises cyclosporin A, Chemal TDA-9®, and Lavender oil.

Another embodiment comprises cyclosporin A, Chemax CO-16®, and Lavender oil.

Another embodiment comprises cyclosporin A, Chemax CO-200/50®, and Lavender oil.

Another embodiment comprises cyclosporin A, Chemax CO-25®, and Lavender oil.

Another embodiment comprises cyclosporin A, Chemax CO-30®, and Lavender oil.

Another embodiment comprises cyclosporin A, Chemax CO-36®, and Lavender oil.

Another embodiment comprises cyclosporin A, Chemax CO-40®, and Lavender oil.

Another embodiment comprises cyclosporin A, Chemax CO-5®, and Lavender oil.

Another embodiment comprises cyclosporin A, Chemax CO-80®, and Lavender oil.

Another embodiment comprises cyclosporin A, Chemax DNP-150/50®, and Lavender oil.

Another embodiment comprises cyclosporin A, Chemax DNP-18®, and Lavender oil.

Another embodiment comprises cyclosporin A, Chemax DNP-8®, and Lavender oil.

Another embodiment comprises cyclosporin A, Chemax E 200 ML®, and Lavender oil.

Another embodiment comprises cyclosporin A, Chemax E 200 MO®, and Lavender oil.

Another embodiment comprises cyclosporin A, Chemax E 400 ML®, and Lavender oil.

Another embodiment comprises cyclosporin A, Chemax E 400 MO®, and Lavender oil.

Another embodiment comprises cyclosporin A, Chemax E 600 ML®, and Lavender oil.

Another embodiment comprises cyclosporin A, Chemax E 600 MO®, and Lavender oil.

Another embodiment comprises cyclosporin A, Chemax E 1000 MO®, and Lavender oil.

Another embodiment comprises cyclosporin A, Chemax E 1000 MS®, and Lavender oil.

Another embodiment comprises cyclosporin A, Chemax E200 MS®, and Lavender oil.

Another embodiment comprises cyclosporin A, Chemax E400 MS®, and Lavender oil.

Another embodiment comprises cyclosporin A, Chemax E600 MS®, and Lavender oil.

Another embodiment comprises cyclosporin A, Chemax HCO-16®, and Lavender oil.

Another embodiment comprises cyclosporin A, Chemax HCO-200/50®, and Lavender oil.

Another embodiment comprises cyclosporin A, Chemax HCO-25®, and Lavender oil.

Another embodiment comprises cyclosporin A, Chemax HCO-5®, and Lavender oil.

Another embodiment comprises cyclosporin A, Chemax TO-16®, and Lavender oil.

Another embodiment comprises cyclosporin A, Chemax TO-8®, and Lavender oil.

Another embodiment comprises cyclosporin A, Colalube 3409®, and Lavender oil.

Another embodiment comprises cyclosporin A, Colalube 3411®, and Lavender oil.

Another embodiment comprises cyclosporin A, Colalube 3413®, and Lavender oil.

Another embodiment comprises cyclosporin A, Colalube 3414®, and Lavender oil.

Another embodiment comprises cyclosporin A, Colalube 3415®, and Lavender oil.

Another embodiment comprises cyclosporin A, Colalube 3416®, and Lavender oil.

Another embodiment comprises cyclosporin A, Colalube 3417®, and Lavender oil.

Another embodiment comprises cyclosporin A, Delonic NPE-100®, and Lavender oil.

Another embodiment comprises cyclosporin A, Delonic NPE-30®, and Lavender oil.

Another embodiment comprises cyclosporin A, Delonic NPE-40®, and Lavender oil.

Another embodiment comprises cyclosporin A, Delonic OPE-10®, and Lavender oil.

Another embodiment comprises cyclosporin A, Delonic OPE-12®, and Lavender oil.

Another embodiment comprises cyclosporin A, Delonic OPE-30®, and Lavender oil.

Another embodiment comprises cyclosporin A, Delonic OPE-40®, and Lavender oil.

Another embodiment comprises cyclosporin A, Delonic OPE-5®, and Lavender oil.

Another embodiment comprises cyclosporin A, Delonic OPE-7.5®, and Lavender oil.

Another embodiment comprises cyclosporin A, DePeg 16-CO®, and Lavender oil.

Another embodiment comprises cyclosporin A, DePeg 25-CO®, and Lavender oil.

Another embodiment comprises cyclosporin A, DePeg 30-CO®, and Lavender oil.

Another embodiment comprises cyclosporin A, DePeg 40-CO®, and Lavender oil.

Another embodiment comprises cyclosporin A, DePeg 5-CO®, and Lavender oil.

Another embodiment comprises cyclosporin A, DePeg 80-CO®, and Lavender oil.

Another embodiment comprises cyclosporin A, DeSonic S-100®, and Lavender oil.

Another embodiment comprises cyclosporin A, DeSonic S-114®, and Lavender oil.

Another embodiment comprises cyclosporin A, DeSonic S-405®, and Lavender oil.

Another embodiment comprises cyclosporin A, DeSonic S-45®, and Lavender oil.

Another embodiment comprises cyclosporin A, DeSonic TDA-9®, and Lavender oil.

Another embodiment comprises cyclosporin A, DeThox Acid O-14®, and Lavender oil.

Another embodiment comprises cyclosporin A, DeThox Acid O-16®, and Lavender oil.

Another embodiment comprises cyclosporin A, DeThox Acid O-9®, and Lavender oil.

Another embodiment comprises cyclosporin A, DeThox Acid TO-14®, and Lavender oil.

Another embodiment comprises cyclosporin A, DeThox Acid TO-16.5®, and Lavender oil.

Another embodiment comprises cyclosporin A, DeThox Acid TO-8.5®, and Lavender oil.

Another embodiment comprises cyclosporin A, Dur-Em 114®, and Lavender oil.

Another embodiment comprises cyclosporin A, Dur-Em 117®, and Lavender oil.

Another embodiment comprises cyclosporin A, Dur-Em 204®, and Lavender oil.

Another embodiment comprises cyclosporin A, Dur-Em 207®, and Lavender oil.

Another embodiment comprises cyclosporin A, Dur-Em 207E®, and Lavender oil.

Another embodiment comprises cyclosporin A, Emerest 2704®, and Lavender oil.

Another embodiment comprises cyclosporin A, Emerest 2712®, and Lavender oil.

Another embodiment comprises cyclosporin A, Emerest 2715®, and Lavender oil.

Another embodiment comprises cyclosporin A, Ethal EH-2®, and Lavender oil.

Another embodiment comprises cyclosporin A, Ethal EH-5®, and Lavender oil.

Another embodiment comprises cyclosporin A, Ethal OA-23®, and Lavender oil.

Another embodiment comprises cyclosporin A, Ethosperse CA-20®, and Lavender oil.

Another embodiment comprises cyclosporin A, Ethosperse LA-12®, and Lavender oil.

Another embodiment comprises cyclosporin A, Ethosperse LA-23®, and Lavender oil.

Another embodiment comprises cyclosporin A, Ethosperse LA-4®, and Lavender oil.

Another embodiment comprises cyclosporin A, Ethox 3095®, and Lavender oil.

Another embodiment comprises cyclosporin A, Ethox CO-16®, and Lavender oil.

Another embodiment comprises cyclosporin A, Ethox CO-200®, and Lavender oil.

Another embodiment comprises cyclosporin A, Ethox CO-25®, and Lavender oil.

Another embodiment comprises cyclosporin A, Ethox CO-30®, and Lavender oil.

Another embodiment comprises cyclosporin A, Ethox CO-36®, and Lavender oil.

Another embodiment comprises cyclosporin A, Ethox CO-40®, and Lavender oil.

Another embodiment comprises cyclosporin A, Ethox CO-5®, and Lavender oil.

Another embodiment comprises cyclosporin A, Ethox DO-14®, and Lavender oil.

Another embodiment comprises cyclosporin A, Ethox DO-9®, and Lavender oil.

Another embodiment comprises cyclosporin A, Ethox MA-8®, and Lavender oil.

Another embodiment comprises cyclosporin A, Ethox MI-14®, and Lavender oil.

Another embodiment comprises cyclosporin A, Ethox ML-14®, and Lavender oil.

Another embodiment comprises cyclosporin A, Ethox ML-5®, and Lavender oil.

Another embodiment comprises cyclosporin A, Ethox MO-14®, and Lavender oil.

Another embodiment comprises cyclosporin A, Ethox MO-9®, and Lavender oil.

Another embodiment comprises cyclosporin A, Ethox MS-14®, and Lavender oil.

Another embodiment comprises cyclosporin A, Ethox MS-23®, and Lavender oil.

Another embodiment comprises cyclosporin A, Ethox MS-40®, and Lavender oil.

Another embodiment comprises cyclosporin A, Ethox MS-8®, and Lavender oil.

Another embodiment comprises cyclosporin A, Ethox TO-16®, and Lavender oil.

Another embodiment comprises cyclosporin A, Ethox TO-8®, and Lavender oil.

Another embodiment comprises cyclosporin A, Ethylan SN-120®, and Lavender oil.

Another embodiment comprises cyclosporin A, Ethylan SN-70®, and Lavender oil.

Another embodiment comprises cyclosporin A, Ethylan SN-90®, and Lavender oil.

Another embodiment comprises cyclosporin A, Flexricin 13®, and Lavender oil.

Another embodiment comprises cyclosporin A, Flexricin 15®, and Lavender oil.

Another embodiment comprises cyclosporin A, Genapol 1261®, and Lavender oil.

Another embodiment comprises cyclosporin A, Generol 122 E25®, and Lavender oil.

Another embodiment comprises cyclosporin A, Glicepol 160®, and Lavender oil.

Another embodiment comprises cyclosporin A, Glicepol 560®, and Lavender oil.

Another embodiment comprises cyclosporin A, Glicepol GMS 20®, and Lavender oil.

Another embodiment comprises cyclosporin A, Hetoxide BN-13®, and Lavender oil.

Another embodiment comprises cyclosporin A, Hetoxide DNP-4®, and Lavender oil.

Another embodiment comprises cyclosporin A, Hetoxide DNP-9.6®, and Lavender oil.

Another embodiment comprises cyclosporin A, Hetoxide G-26®, and Lavender oil.

Another embodiment comprises cyclosporin A, Hetoxide G-7®, and Lavender oil.

Another embodiment comprises cyclosporin A, Hetoxide GT-20®, and Lavender oil.

Another embodiment comprises cyclosporin A, Hetoxide HC-40®, and Lavender oil.

Another embodiment comprises cyclosporin A, Hetoxol C-24®, and Lavender oil.

Another embodiment comprises cyclosporin A, Hetoxol CA-10®, and Lavender oil.

Another embodiment comprises cyclosporin A, Hetoxol CA-2®, and Lavender oil.

Another embodiment comprises cyclosporin A, Hetoxol CA-20®, and Lavender oil.

Another embodiment comprises cyclosporin A, Hetoxol CAWS®, and Lavender oil.

Another embodiment comprises cyclosporin A, Hetoxol CD-3®, and Lavender oil.

Another embodiment comprises cyclosporin A, Hetoxol CD-4®, and Lavender oil.

Another embodiment comprises cyclosporin A, Hetoxol CS-15®, and Lavender oil.

Another embodiment comprises cyclosporin A, Hetoxol CS-20®, and Lavender oil.

Another embodiment comprises cyclosporin A, Hetoxdl CS-20D®, and Lavender oil.

Another embodiment comprises cyclosporin A, Hetoxol CS-25®, and Lavender oil.

Another embodiment comprises cyclosporin A, Hetoxol CS-50®, and Lavender oil.

Another embodiment comprises cyclosporin A, Hetoxol CS-9®, and Lavender oil.

Another embodiment comprises cyclosporin A, Hetoxol CSA-15®, and Lavender oil.

Another embodiment comprises cyclosporin A, Hetoxol D®, and Lavender oil.

Another embodiment comprises cyclosporin A, Hetoxol G®, and Lavender oil.

Another embodiment comprises cyclosporin A, Hetoxol J®, and Lavender oil.

Another embodiment comprises cyclosporin A, Hetoxol L®, and Lavender oil.

Another embodiment comprises cyclosporin A, Hetoxol L-23®, and Lavender oil.

Another embodiment comprises cyclosporin A, Hetoxol L-4®, and Lavender oil.

Another embodiment comprises cyclosporin A, Hetoxol L-9®, and Lavender oil.

Another embodiment comprises cyclosporin A, Hetoxol LS-9®, and Lavender oil.

Another embodiment comprises cyclosporin A, Hetoxol M-3®, and Lavender oil.

Another embodiment comprises cyclosporin A, Hetoxol OA-10 Special®, and Lavender oil.

Another embodiment comprises cyclosporin A, Hetoxol OA-20 Special®, and Lavender oil.

Another embodiment comprises cyclosporin A, Hetoxol OA-3 Special®, and Lavender oil.

Another embodiment comprises cyclosporin A, Hetoxol OA-5 Special®, and Lavender oil.

Another embodiment comprises cyclosporin A, Hetoxol OL-10®, and Lavender oil.

Another embodiment comprises cyclosporin A, Hetoxol OL-2®, and Lavender oil.

Another embodiment comprises cyclosporin A, Hetoxol OL-23®, and Lavender oil.

Another embodiment comprises cyclosporin A, Hetoxol OL-4®, and Lavender oil.

Another embodiment comprises cyclosporin A, Hetoxol OL-40®, and Lavender oil.

Another embodiment comprises cyclosporin A, Hetoxol OL-5®, and Lavender oil.

Another embodiment comprises cyclosporin A, Hetoxol PLA®, and Lavender oil.

Another embodiment comprises cyclosporin A, Hetoxol SP-15®, and Lavender oil.

Another embodiment comprises cyclosporin A, Hetoxol STA-10®, and Lavender oil.

Another embodiment comprises cyclosporin A, Hetoxol STA-2®, and Lavender oil.

Another embodiment comprises cyclosporin A, Hetoxol STA-30®, and Lavender oil.

Another embodiment comprises cyclosporin A, Hetoxol TD-12®, and Lavender oil.

Another embodiment comprises cyclosporin A, Hetoxol TD-18®, and Lavender oil.

Another embodiment comprises cyclosporin A, Hetoxol TD-3®, and Lavender oil.

Another embodiment comprises cyclosporin A, Hetoxol TD-6®, and Lavender oil.

Another embodiment comprises cyclosporin A, Hetoxol TD-9®, and Lavender oil.

Another embodiment comprises cyclosporin A, Hetoxol TDEP-15®, and Lavender oil.

Another embodiment comprises cyclosporin A, Hetoxol TDEP-63®, and Lavender oil.

Another embodiment comprises cyclosporin A, Iconol 24-12®, and Lavender oil.

Another embodiment comprises cyclosporin A, lconol 24-23®, and Lavender oil.

Another embodiment comprises cyclosporin A, Iconol 24-4®, and Lavender oil.

Another embodiment comprises cyclosporin A, Iconol DA-4®, and Lavender oil.

Another embodiment comprises cyclosporin A, Iconol DA-6®, and Lavender oil.

Another embodiment comprises cyclosporin A, Iconol TDA-10®, and Lavender oil.

Another embodiment comprises cyclosporin A, Iconol TDA-3®, and Lavender oil.

Another embodiment comprises cyclosporin A, Iconol TDA-6®, and Lavender oil.

Another embodiment comprises cyclosporin A, Iconol TDA-8-90%®, and Lavender oil.

Another embodiment comprises cyclosporin A, Iconol TDA-9®, and Lavender oil.

Another embodiment comprises cyclosporin A, Igepal CA-210®, and Lavender oil.

Another embodiment comprises cyclosporin A, Igepal CA-407®, and Lavender oil.

Another embodiment comprises cyclosporin A, Igepal CA-420®, and Lavender oil.

Another embodiment comprises cyclosporin A, Igepal CA-520®, and Lavender oil.

Another embodiment comprises cyclosporin A, Igepal CA-620®, and Lavender oil.

Another embodiment comprises cyclosporin A, Igepal CA-630®, and Lavender oil.

Another embodiment comprises cyclosporin A, Igepal CA-720®, and Lavender oil.

Another embodiment comprises cyclosporin A, Igepal CA-877®, and Lavender oil.

Another embodiment comprises cyclosporin A, Igepal CA-887®, and Lavender oil.

Another embodiment comprises cyclosporin A, Igepal CA-897®, and Lavender oil.

Another embodiment comprises cyclosporin A, Igepal CO-210®, and Lavender oil.

Another embodiment comprises cyclosporin A, Igepal CO-430®, and Lavender oil.

Another embodiment comprises cyclosporin A, Igepal CO-520®, and Lavender oil.

Another embodiment comprises cyclosporin A, Igepal CO-530®, and Lavender oil.

Another embodiment comprises cyclosporin A, Igepal CO-610®, and Lavender oil.

Another embodiment comprises cyclosporin A, Igepal CO-630®, and Lavender oil.

Another embodiment comprises cyclosporin A, Igepal CO-630 Special®, and Lavender oil.

Another embodiment comprises cyclosporin A, Igepal CO-660®, and Lavender oil.

Another embodiment comprises cyclosporin A, Igepal CO-710®, and Lavender oil.

Another embodiment comprises cyclosporin A, Igepal CO-720®, and Lavender oil.

Another embodiment comprises cyclosporin A, Igepal CO-730®, and Lavender oil.

Another embodiment comprises cyclosporin A, Igepal CO-738®, and Lavender oil.

Another embodiment comprises cyclosporin A, Igepal CO-850®, and Lavender oil.

Another embodiment comprises cyclosporin A, Igepal CO-880®, and Lavender oil.

Another embodiment comprises cyclosporin A, Igepal CO-887®, and Lavender oil.

Another embodiment comprises cyclosporin A, Igepal CO-890®, and Lavender oil.

Another embodiment comprises cyclosporin A, Igepal CO-897®, and Lavender oil.

Another embodiment comprises cyclosporin A, Igepal CO-970®, and Lavender oil.

Another embodiment comprises cyclosporin A, Igepal CO-977®, and Lavender oil.

Another embodiment comprises cyclosporin A, Igepal CO-987®, and Lavender oil.

Another embodiment comprises cyclosporin A, Igepal CO-990 FLK®, and Lavender oil.

Another embodiment comprises cyclosporin A, Igepal CO-997®, and Lavender oil.

Another embodiment comprises cyclosporin A, Igepal DM-430®, and Lavender oil.

Another embodiment comprises cyclosporin A, Igepal DM-530®, and Lavender oil.

Another embodiment comprises cyclosporin A, Igepal DM-710®, and Lavender oil.

Another embodiment comprises cyclosporin A, Igepal DM-730®, and Lavender oil.

Another embodiment comprises cyclosporin A, Igepal DM-880®, and Lavender oil.

Another embodiment comprises cyclosporin A, Igepal DM-970 FLK®, and Lavender oil.

Another embodiment comprises cyclosporin A, Igepal OD-410®, and Lavender oil.

Another embodiment comprises cyclosporin A, Igepal RC-520®, and Lavender oil.

Another embodiment comprises cyclosporin A, Igepal RC-620®, and Lavender oil.

Another embodiment comprises cyclosporin A, Igepal RC-630®, and Lavender oil.

Another embodiment comprises cyclosporin A, Lexemul CS-20®, and Lavender oil.

Another embodiment comprises cyclosporin A, Lipo GMS 450®, and Lavender oil.

Another embodiment comprises cyclosporin A, Lipo Polyglycol 1000®, and Lavender oil.

Another embodiment comprises cyclosporin A, Lipo Polyglycol 200®, and Lavender oil.

Another embodiment comprises cyclosporin A, Lipo Polyglycol 300®, and Lavender oil.

Another embodiment comprises cyclosporin A, Lipo Polyglycol 3350®, and Lavender oil.

Another embodiment comprises cyclosporin A, Lipo Polyglycol 400®, and Lavender oil.

Another embodiment comprises cyclosporin A, Lipo Polyglycol 600®, and Lavender oil.

Another embodiment comprises cyclosporin A, Lipocol C-10®, and Lavender oil.

Another embodiment comprises cyclosporin A, Lipocol C-2®, and Lavender oil.

Another embodiment comprises cyclosporin A, Lipocol C-20®, and Lavender oil.

Another embodiment comprises cyclosporin A, Lipocol L-12®, and Lavender oil.

Another embodiment comprises cyclosporin A, Lipocol L-23®, and Lavender oil.

Another embodiment comprises cyclosporin A, Lipocol L-4®, and Lavender oil.

Another embodiment comprises cyclosporin A, Lipocol O-10®, and Lavender oil.

Another embodiment comprises cyclosporin A, Lipocol O-2®, and Lavender oil.

Another embodiment comprises cyclosporin A, Lipocol O-20®, and Lavender oil.

Another embodiment comprises cyclosporin A, Lipocol O-5®, and Lavender oil.

Another embodiment comprises cyclosporin A, Lipocol S-10®, and Lavender oil.

Another embodiment comprises cyclosporin A, Lipocol S-2®, and Lavender oil.

Another embodiment comprises cyclosporin A, Lipocol S-20®, and Lavender oil.

Another embodiment comprises cyclosporin A, Lipocol SC-15®, and Lavender oil.

Another embodiment comprises cyclosporin A, Lipocol SC-20®, and Lavender oil.

Another embodiment comprises cyclosporin A, Lipocol SC-4®, and Lavender oil.

Another embodiment comprises cyclosporin A, Lipocol TD-12®, and Lavender oil.

Another embodiment comprises cyclosporin A, Lumulse 40-O K®, and Lavender oil.

Another embodiment comprises cyclosporin A, Lumulse 42-O K®, and Lavender oil.

Another embodiment comprises cyclosporin A, Lumulse GMT-40®, and Lavender oil.

Another embodiment comprises cyclosporin A, Lumulse PEG 1450®, and Lavender oil.

Another embodiment comprises cyclosporin A, Lumulse PEG 1450 NF®, and Lavender oil.

Another embodiment comprises cyclosporin A, Lumulse PEG 8000®, and Lavender oil.

Another embodiment comprises cyclosporin A, Lumulse PGO®, and Lavender oil.

Another embodiment comprises cyclosporin A, Lumulse POE (2) Oleyl Amine®, and Lavender oil.

Another embodiment comprises cyclosporin A, Magrabar PGO-104®, and Lavender oil.

Another embodiment comprises cyclosporin A, Mapeg 200 ML®, and Lavender oil.

Another embodiment comprises cyclosporin A, Mapeg 400 MO®, and Lavender oil.

Another embodiment comprises cyclosporin A, Mapeg 400 MOT®, and Lavender oil.

Another embodiment comprises cyclosporin A, Mapeg 400DO®, and Lavender oil.

Another embodiment comprises cyclosporin A, Mapeg 400DOT®, and Lavender oil.

Another embodiment comprises cyclosporin A, Mapeg 600DOT®, and Lavender oil.

Another embodiment comprises cyclosporin A, Marlox B 24/80®, and Lavender oil.

Another embodiment comprises cyclosporin A, Marlox FK 86®, and Lavender oil.

Another embodiment comprises cyclosporin A, Marlox MO 154®, and Lavender oil.

Another embodiment comprises cyclosporin A, Mazox CDA CG®, and Lavender oil.

Another embodiment comprises cyclosporin A, Monoglycerides Glyceryl Monestearate®, and Lavender oil.

Another embodiment comprises cyclosporin A, Myrj 45®, and Lavender oil.

Another embodiment comprises cyclosporin A, Myrj 52®, and Lavender oil.

Another embodiment comprises cyclosporin A, Myrj 52S®, and Lavender oil.

Another embodiment comprises cyclosporin A, Myrj 53®, and Lavender oil.

Another embodiment comprises cyclosporin A, Myrj 59®, and Lavender oil.

Another embodiment comprises cyclosporin A, Norfox NP-12®, and Lavender oil.

Another embodiment comprises cyclosporin A, Norfox NP-4®, and Lavender oil.

Another embodiment comprises cyclosporin A, Norfox NP-6®, and Lavender oil.

Another embodiment comprises cyclosporin A, Norfox NP-887®, and Lavender oil.

Another embodiment comprises cyclosporin A, Norfox NP-9®, and Lavender oil.

Another embodiment comprises cyclosporin A, Norfox NP-977®, and Lavender oil.

Another embodiment comprises cyclosporin A, Norfox OP-100®, and Lavender oil.

Another embodiment comprises cyclosporin A, Norfox OP-102®, and Lavender oil.

Another embodiment comprises cyclosporin A, Norfox OP-114®, and Lavender oil.

Another embodiment comprises cyclosporin A, Norfox OP-45®, and Lavender oil.

Another embodiment comprises cyclosporin A, Pegosperse 100-L®, and Lavender oil.

Another embodiment comprises cyclosporin A, Pegosperse 100-O®, and Lavender oil.

Another embodiment comprises cyclosporin A, Pegosperse 100-S®, and Lavender oil.

Another embodiment comprises cyclosporin A, Pegosperse 1500-MS®, and Lavender oil.

Another embodiment comprises cyclosporin A, Pegosperse 1750-MS®, and Lavender oil.

Another embodiment comprises cyclosporin A, Pegosperse 200 DL®, and Lavender oil.

Another embodiment comprises cyclosporin A, Pegosperse 200-ML®, and Lavender oil.

Another embodiment comprises cyclosporin A, Pegosperse 400-DL®, and Lavender oil.

Another embodiment comprises cyclosporin A, Pegosperse 400-DO®, and Lavender oil.

Another embodiment comprises cyclosporin A, Pegosperse 400-DS®, and Lavender oil.

Another embodiment comprises cyclosporin A, Pegosperse 400-ML®, and Lavender oil.

Another embodiment comprises cyclosporin A, Pegosperse 400-MO®, and Lavender oil.

Another embodiment comprises cyclosporin A, Pegosperse 400-MOT®, and Lavender oil.

Another embodiment comprises cyclosporin A, Pegosperse 400-MS®, and Lavender oil.

Another embodiment comprises cyclosporin A, Pegosperse 50-DS®, and Lavender oil.

Another embodiment comprises cyclosporin A, Pegosperse 50-MS®, and Lavender oil.

Another embodiment comprises cyclosporin A, Pegosperse 600-ML®, and Lavender oil.

Another embodiment comprises cyclosporin A, Pegosperse 600-MS®, and Lavender oil.

Another embodiment comprises cyclosporin A, Plurafac A-38®, and Lavender oil.

Another embodiment comprises cyclosporin A, Plurafac B-25-5®, and Lavender oil.

Another embodiment comprises cyclosporin A, Plurafac B-26®, and Lavender oil.

Another embodiment comprises cyclosporin A, Plurafac D-25®, and Lavender oil.

Another embodiment comprises cyclosporin A, Plurafac LF 131®, and Lavender oil.

Another embodiment comprises cyclosporin A, Plurafac LF 4030®, and Lavender oil.

Another embodiment comprises cyclosporin A, Plurafac LF 7000®, and Lavender oil.

Another embodiment comprises cyclosporin A, Plurafac RA-20®, and Lavender oil.

Another embodiment comprises cyclosporin A, Plurafac RA-30®, and Lavender oil.

Another embodiment comprises cyclosporin A, Plurafac RA-40®, and Lavender oil.

Another embodiment comprises cyclosporin A, Pluronic 10R5®, and Lavender oil.

Another embodiment comprises cyclosporin A, Pluronic 17R2®, and Lavender oil.

Another embodiment comprises cyclosporin A, Pluronic 17R4®, and Lavender oil.

Another embodiment comprises cyclosporin A, Pluronic 25R2®, and Lavender oil.

Another embodiment comprises cyclosporin A, Pluronic 25R4®, and Lavender oil.

Another embodiment comprises cyclosporin A, Pluronic 31R1®, and Lavender oil.

Another embodiment comprises cyclosporin A, Pluronic F108®, and Lavender oil.

Another embodiment comprises cyclosporin A, Pluronic F127®, and Lavender oil.

Another embodiment comprises cyclosporin A, Pluronic F38®, and Lavender oil.

Another embodiment comprises cyclosporin A, Pluronic F68®, and Lavender oil.

Another embodiment comprises cyclosporin A, Pluronic F68LF®, and Lavender oil.

Another embodiment comprises cyclosporin A, Pluronic F77®, and Lavender oil.

Another embodiment comprises cyclosporin A, Pluronic F87®, and Lavender oil.

Another embodiment comprises cyclosporin A, Pluronic F88®, and Lavender oil.

Another embodiment comprises cyclosporin A, Pluronic F98®, and Lavender oil.

Another embodiment comprises cyclosporin A, Pluronic L10®, and Lavender oil.

Another embodiment comprises cyclosporin A, Pluronic L101®, and Lavender oil.

Another embodiment comprises cyclosporin A, Pluronic L121®, and Lavender oil.

Another embodiment comprises cyclosporin A, Pluronic L31®, and Lavender oil.

Another embodiment comprises cyclosporin A, Pluronic L35®, and Lavender oil.

Another embodiment comprises cyclosporin A, Pluronic L43®, and Lavender oil.

Another embodiment comprises cyclosporin A, Pluronic L44®, and Lavender oil.

Another embodiment comprises cyclosporin A, Pluronic L61®, and Lavender oil.

Another embodiment comprises cyclosporin A, Pluronic L62®, and Lavender oil.

Another embodiment comprises cyclosporin A, Pluronic L62D®, and Lavender oil.

Another embodiment comprises cyclosporin A, Pluronic L62LF®, and Lavender oil.

Another embodiment comprises cyclosporin A, Pluronic L64®, and Lavender oil.

Another embodiment comprises cyclosporin A, Pluronic L81®, and Lavender oil.

Another embodiment comprises cyclosporin A, Pluronic L92®, and Lavender oil.

Another embodiment comprises cyclosporin A, Pluronic P103®, and Lavender oil.

Another embodiment comprises cyclosporin A, Pluronic P104®, and Lavender oil.

Another embodiment comprises cyclosporin A, Pluronic P105®, and Lavender oil.

Another embodiment comprises cyclosporin A, Pluronic P123®, and Lavender oil.

Another embodiment comprises cyclosporin A, Pluronic P65®, and Lavender oil.

Another embodiment comprises cyclosporin A, Pluronic P84®, and Lavender oil.

Another embodiment comprises cyclosporin A, Pluronic P85®, and Lavender oil.

Another embodiment comprises cyclosporin A, Polyaldo 10-2-P®, and Lavender oil.

Another embodiment comprises cyclosporin A, Polyaldo DGDO®, and Lavender oil.

Another embodiment comprises cyclosporin A, Polyaldo HGDS®, and Lavender oil.

Another embodiment comprises cyclosporin A, Polyaldo TGMS®, and Lavender oil.

Another embodiment comprises cyclosporin A, Protegin V®, and Lavender oil.

Another embodiment comprises cyclosporin A, Protegin W®, and Lavender oil.

Another embodiment comprises cyclosporin A, Protegin WX®, and Lavender oil.

Another embodiment comprises cyclosporin A, Protegin XV®, and Lavender oil.

Another embodiment comprises cyclosporin A, Rhodasurf BC-420®, and Lavender oil.

Another embodiment comprises cyclosporin A, Rhodasurf BC-610®, and Lavender oil.

Another embodiment comprises cyclosporin A, Rhodasurf BC-630®, and Lavender oil.

Another embodiment comprises cyclosporin A, Rhodasurf BC-720®, and Lavender oil.

Another embodiment comprises cyclosporin A, Rhodasurf BC-840®, and Lavender oil.

Another embodiment comprises cyclosporin A, Rhodasurf L 1®, and Lavender oil.

Another embodiment comprises cyclosporin A, Rhodasurf L 12®, and Lavender oil.

Another embodiment comprises cyclosporin A, Rhodasurf L 2®, and Lavender oil.

Another embodiment comprises cyclosporin A, Rhodasurf L 20®, and Lavender oil.

Another embodiment comprises cyclosporin A, Rhodasurf L 3®, and Lavender oil.

Another embodiment comprises cyclosporin A, Rhodasurf L 30®, and Lavender oil.

Another embodiment comprises cyclosporin A, Rhodasurf L 4®, and Lavender oil.

Another embodiment comprises cyclosporin A, Rhodasurf L 50®, and Lavender oil.

Another embodiment comprises cyclosporin A, Rhodasurf L 7/90®, and Lavender oil.

Another embodiment comprises cyclosporin A, Rhodasurf LA-3®, and Lavender oil.

Another embodiment comprises cyclosporin A, Rhodasurf LA-7®, and Lavender oil.

Another embodiment comprises cyclosporin A, Rhodasurf LAN-23®, and Lavender oil.

Another embodiment comprises cyclosporin A, Rhodasurf ON-870®, and Lavender oil.

Another embodiment comprises cyclosporin A, Rhodasurf ON-877®, and Lavender oil.

Another embodiment comprises cyclosporin A, Rhodasurf TDA-8/5®, and Lavender oil.

Another embodiment comprises cyclosporin A, Ritapro 100®, and Lavender oil.

Another embodiment comprises cyclosporin A, Ritapro 165®, and Lavender oil.

Another embodiment comprises cyclosporin A, Ritapro 200®, and Lavender oil.

Another embodiment comprises cyclosporin A, Ritapro 300®, and Lavender oil.

Another embodiment comprises cyclosporin A, Ritoleth 10®, and Lavender oil.

Another embodiment comprises cyclosporin A, Ritoleth 2®, and Lavender oil.

Another embodiment comprises cyclosporin A, Ritoleth 20®, and Lavender oil.

Another embodiment comprises cyclosporin A, Ritoleth 5®, and Lavender oil.

Another embodiment comprises cyclosporin A, Santone 10-10-0®, and Lavender oil.

Another embodiment comprises cyclosporin A, Santone 3-1-S XTR®, and Lavender oil.

Another embodiment comprises cyclosporin A, Santone 3-1-SH®, and Lavender oil.

Another embodiment comprises cyclosporin A, Santone 8-1-O®, and Lavender oil.

Another embodiment comprises cyclosporin A, Solulan 16®, and Lavender oil.

Another embodiment comprises cyclosporin A, Solulan 75®, and Lavender oil.

Another embodiment comprises cyclosporin A, Solulan C-24®, and Lavender oil.

Another embodiment comprises cyclosporin A, Solulan L-575®, and Lavender oil.

Another embodiment comprises cyclosporin A, Standamox CAW®, and Lavender oil.

Another embodiment comprises cyclosporin A, Sunine AZI-A®, and Lavender oil.

Another embodiment comprises cyclosporin A, Surfonic CO-15®, and Lavender oil.

Another embodiment comprises cyclosporin A, Surfonic CO-25®, and Lavender oil.

Another embodiment comprises cyclosporin A, Surfonic CO-30®, and Lavender oil.

Another embodiment comprises cyclosporin A, Surfonic CO-36®, and Lavender oil.

Another embodiment comprises cyclosporin A, Surfonic CO-42®, and Lavender oil.

Another embodiment comprises cyclosporin A, Surfonic E400-MO®, and Lavender oil.

Another embodiment comprises cyclosporin A, Surfonic L12-3®, and Lavender oil.

Another embodiment comprises cyclosporin A, Surfonic L12-6®, and Lavender oil.

Another embodiment comprises cyclosporin A, Surfonic L12-8®, and Lavender oil.

Another embodiment comprises cyclosporin A, Surfonic L24-12®, and Lavender oil.

Another embodiment comprises cyclosporin A, Surfonic L24-2®, and Lavender oil.

Another embodiment comprises cyclosporin A, Surfonic L24-22®, and Lavender oil.

Another embodiment comprises cyclosporin A, Surfonic L24-3®, and Lavender oil.

Another embodiment comprises cyclosporin A, Surfonic L24-4®, and Lavender oil.

Another embodiment comprises cyclosporin A, Surfonic L24-5®, and Lavender oil.

Another embodiment comprises cyclosporin A, Surfonic L24-7®, and Lavender oil.

Another embodiment comprises cyclosporin A, Surfonic L24-7.1®, and Lavender oil.

Another embodiment comprises cyclosporin A, Surfonic L24-9®, and Lavender oil.

Another embodiment comprises cyclosporin A, Surfonic LF-17®, and Lavender oil.

Another embodiment comprises cyclosporin A, Surfonic LF-18®, and Lavender oil.

Another embodiment comprises cyclosporin A, Surfonic LF-37®, and Lavender oil.

Another embodiment comprises cyclosporin A, Surfonic LF-41®, and Lavender oil.

Another embodiment comprises cyclosporin A, Surfonic LF-42®, and Lavender oil.

Another embodiment comprises cyclosporin A, Surfonic LF-47®, and Lavender oil.

Another embodiment comprises cyclosporin A, Surfonic LF-68®, and Lavender oil.

Another embodiment comprises cyclosporin A, Surfonic N-10®, and Lavender oil.

Another embodiment comprises cyclosporin A, Surfonic N-100®, and Lavender oil.

Another embodiment comprises cyclosporin A, Surfonic N-102®, and Lavender oil.

Another embodiment comprises cyclosporin A, Surfonic N-120®, and Lavender oil.

Another embodiment comprises cyclosporin A, Surfonic N-150®, and Lavender oil.

Another embodiment comprises cyclosporin A, Surfonic N-200®, and Lavender oil.

Another embodiment comprises cyclosporin A, Surfonic N-300®, and Lavender oil.

Another embodiment comprises cyclosporin A, Surfonic N-31.5®, and Lavender oil.

Another embodiment comprises cyclosporin A, Surfonic N-40®, and Lavender oil.

Another embodiment comprises cyclosporin A, Surfonic N-400®, and Lavender oil.

Another embodiment comprises cyclosporin A, Surfonic N-60®, and Lavender oil.

Another embodiment comprises cyclosporin A, Surfonic N-85®, and Lavender oil.

Another embodiment comprises cyclosporin A, Surfonic N-95®, and Lavender oil.

Another embodiment comprises cyclosporin A, Surfonic NB-18®, and Lavender oil.

Another embodiment comprises cyclosporin A, Surfonic NB-23®, and Lavender oil.

Another embodiment comprises cyclosporin A, Surfonic NB-25®, and Lavender oil.

Another embodiment comprises cyclosporin A, Surfonic NB-307®, and Lavender oil.

Another embodiment comprises cyclosporin A, Surfonic NB-31®, and Lavender oil.

Another embodiment comprises cyclosporin A, Surfonic NB-41®, and Lavender oil.

Another embodiment comprises cyclosporin A, Surfonic NB-43®, and Lavender oil.

Another embodiment comprises cyclosporin A, Surfonic OP-100®, and Lavender oil.

Another embodiment comprises cyclosporin A, Surfonic OP-120®, and Lavender oil.

Another embodiment comprises cyclosporin A, Surfonic OP-15®, and Lavender oil.

Another embodiment comprises cyclosporin A, Surfonic OP-35®, and Lavender oil.

Another embodiment comprises cyclosporin A, Surfonic OP-70®, and Lavender oil.

Another embodiment comprises cyclosporin A, Surfonic P1®, and Lavender oil.

Another embodiment comprises cyclosporin A, Surfonic P3®, and Lavender oil.

Another embodiment comprises cyclosporin A, Surfonic P5®, and Lavender oil.

Another embodiment comprises cyclosporin A, Surfonic P6®, and Lavender oil.

Another embodiment comprises cyclosporin A, Surfonic POA-17R2®, and Lavender oil.

Another embodiment comprises cyclosporin A, Surfonic POA-25R2®, and Lavender oil.

Another embodiment comprises cyclosporin A, Surfonic TDA-11®, and Lavender oil.

Another embodiment comprises cyclosporin A, Surfonic TDA-3B®, and Lavender oil.

Another embodiment comprises cyclosporin A, Surfonic TDA-6®, and Lavender oil.

Another embodiment comprises cyclosporin A, Surfonic TDA-6/88®, and Lavender oil.

Another embodiment comprises cyclosporin A, Surfonic TDA-8®, and Lavender oil.

Another embodiment comprises cyclosporin A, Surfonic TDA-8/90®, and Lavender oil.

Another embodiment comprises cyclosporin A, Surfonic TDA-9®, and Lavender oil.

Another embodiment comprises cyclosporin A, Surfonic TX-CCN®, and Lavender oil.

Another embodiment comprises cyclosporin A, Tagat CH 40®, and Lavender oil.

Another embodiment comprises cyclosporin A, Tagat CH 60®, and Lavender oil.

Another embodiment comprises cyclosporin A, Tagat L 2®, and Lavender oil.

Another embodiment comprises cyclosporin A, Tagat S®, and Lavender oil.

Another embodiment comprises cyclosporin A, Tagat S 2®, and Lavender oil.

Another embodiment comprises cyclosporin A, Tally 100 Plus®, and Lavender oil.

Another embodiment comprises cyclosporin A, T-Det A106®, and Lavender oil.

Another embodiment comprises cyclosporin A, T-Det A109®, and Lavender oil.

Another embodiment comprises cyclosporin A, T-Det A136®, and Lavender oil.

Another embodiment comprises cyclosporin A, T-Det A139®, and Lavender oil.

Another embodiment comprises cyclosporin A, T-Det A2412®, and Lavender oil.

Another embodiment comprises cyclosporin A, T-Det A243®, and Lavender oil.

Another embodiment comprises cyclosporin A, T-Det A247®, and Lavender oil.

Another embodiment comprises cyclosporin A, T-Det A249®, and Lavender oil.

Another embodiment comprises cyclosporin A, T-Det A467®, and Lavender oil.

Another embodiment comprises cyclosporin A, T-Det COE®, and Lavender oil.

Another embodiment comprises cyclosporin A, T-Det DD-10®, and Lavender oil.

Another embodiment comprises cyclosporin A, T-Det DD-5®, and Lavender oil.

Another embodiment comprises cyclosporin A, T-Det DD-7®, and Lavender oil.

Another embodiment comprises cyclosporin A, T-Det EPO-104®, and Lavender oil.

Another embodiment comprises cyclosporin A, T-Det EPO-61®, and Lavender oil.

Another embodiment comprises cyclosporin A, T-Det EPO-62®, and Lavender oil.

Another embodiment comprises cyclosporin A, T-Det EPO-64®, and Lavender oil.

Another embodiment comprises cyclosporin A, T-Det N-1.5®, and Lavender oil.

Another embodiment comprises cyclosporin A, T-Det N-10.5®, and Lavender oil.

Another embodiment comprises cyclosporin A, T-Det N-100®, and Lavender oil.

Another embodiment comprises cyclosporin A, T-Det N-1007®, and Lavender oil.

Another embodiment comprises cyclosporin A, T-Det N-12®, and Lavender oil.

Another embodiment comprises cyclosporin A, T-Det N-14®, and Lavender oil.

Another embodiment comprises cyclosporin A, T-Det N-20®, and Lavender oil.

Another embodiment comprises cyclosporin A, T-Det N-30®, and Lavender oil.

Another embodiment comprises cyclosporin A, T-Det N-307®, and Lavender oil.

Another embodiment comprises cyclosporin A, T-Det N-4®, and Lavender oil.

Another embodiment comprises cyclosporin A, T-Det N-40®, and Lavender oil.

Another embodiment comprises cyclosporin A, T-Det N-407®, and Lavender oil.

Another embodiment comprises cyclosporin A, T-Det N-50®, and Lavender oil.

Another embodiment comprises cyclosporin A, T-Det N-507®, and Lavender oil.

Another embodiment comprises cyclosporin A, T-Det N-6®, and Lavender oil.

Another embodiment comprises cyclosporin A, T-Det N-70®, and Lavender oil.

Another embodiment comprises cyclosporin A, T-Det N-8®, and Lavender oil.

Another embodiment comprises cyclosporin A, T-Det N-9.5®, and Lavender oil.

Another embodiment comprises cyclosporin A, T-Det O-12®, and Lavender oil.

Another embodiment comprises cyclosporin A, T-Det O-165®, and Lavender oil.

Another embodiment comprises cyclosporin A, T-Det O-307®, and Lavender oil.

Another embodiment comprises cyclosporin A, T-Det O-40®, and Lavender oil.

Another embodiment comprises cyclosporin A, T-Det O-407®, and Lavender oil.

Another embodiment comprises cyclosporin A, T-Det O-6®, and Lavender oil.

Another embodiment comprises cyclosporin A, T-Det O-8®, and Lavender oil.

Another embodiment comprises cyclosporin A, T-Det O-9®, and Lavender oil.

Another embodiment comprises cyclosporin A, Tegin®, and Lavender oil.

Another embodiment comprises cyclosporin A, Tergitol 15-S-12®, and Lavender oil.

Another embodiment comprises cyclosporin A, Tergitol 15-S-15®, and Lavender oil.

Another embodiment comprises cyclosporin A, Tergitol 15-S-20®, and Lavender oil.

Another embodiment comprises cyclosporin A, Tergitol 15-S-20 (80AQ)®, and Lavender oil.

Another embodiment comprises cyclosporin A, Tergitol 15-S-3®, and Lavender oil.

Another embodiment comprises cyclosporin A, Tergitol 15-S-30®, and Lavender oil.

Another embodiment comprises cyclosporin A, Tergitol 15-S-40®, and Lavender oil.

Another embodiment comprises cyclosporin A, Tergitol 15-S-40 (70AQ)®, and Lavender oil.

Another embodiment comprises cyclosporin A, Tergitol 15-S-5®, and Lavender oil.

Another embodiment comprises cyclosporin A, Tergitol 15-S-7®, and Lavender oil.

Another embodiment comprises cyclosporin A, Tergitol 15-S-9®, and Lavender oil.

Another embodiment comprises cyclosporin A, Tergitol L-101®, and Lavender oil.

Another embodiment comprises cyclosporin A, Tergitol L-61®, and Lavender oil.

Another embodiment comprises cyclosporin A, Tergitol L-62®, and Lavender oil.

Another embodiment comprises cyclosporin A, Tergitol L-64®, and Lavender oil.

Another embodiment comprises cyclosporin A, Tergitol L-81®, and Lavender oil.

Another embodiment comprises cyclosporin A, Tergitol NP-10®, and Lavender oil.

Another embodiment comprises cyclosporin A, Tergitol NP-11®, and Lavender oil.

Another embodiment comprises cyclosporin A, Tergitol NP-12S, and Lavender oil.

Another embodiment comprises cyclosporin A, Tergitol NP-13®, and Lavender oil.

Another embodiment comprises cyclosporin A, Tergitol NP-15®, and Lavender oil.

Another embodiment comprises cyclosporin A, Tergitol NP-30®, and Lavender oil.

Another embodiment comprises cyclosporin A, Tergitol NP-30 (70 AQ)®, and Lavender oil.

Another embodiment comprises cyclosporin A, Tergitol NP-4®, and Lavender oil.

Another embodiment comprises cyclosporin A, Tergitol NP-40®, and Lavender oil.

Another embodiment comprises cyclosporin A, Tergitol NP-40 (70 AQ)®, and Lavender oil.

Another embodiment comprises cyclosporin A, Tergitol NP-50 (70 AQ)®, and Lavender oil.

Another embodiment comprises cyclosporin A, Tergitol NP-55 (70 AQ)®, and Lavender oil.

Another embodiment comprises cyclosporin A, Tergitol NP-6®, and Lavender oil.

Another embodiment comprises cyclosporin A, Tergitol NP-7®, and Lavender oil.

Another embodiment comprises cyclosporin A, Tergitol NP-70 (70 AQ)®, and Lavender oil.

Another embodiment comprises cyclosporin A, Tergitol NP-8®, and Lavender oil.

Another embodiment comprises cyclosporin A, Tergitol NP-9®, and Lavender oil.

Another embodiment comprises cyclosporin A, Tergitol NP-9.5®, and Lavender oil.

Another embodiment comprises cyclosporin A, Tergitol TMN-10®, and Lavender oil.

Another embodiment comprises cyclosporin A, Tergitol TMN-3®, and Lavender oil.

Another embodiment comprises cyclosporin A, Tergitol TMN-6®, and Lavender oil.

Another embodiment comprises cyclosporin A, Tergitol XD®, and Lavender oil.

Another embodiment comprises cyclosporin A, Tergitol XH®, and Lavender oil.

Another embodiment comprises cyclosporin A, Tergitol XJ®, and Lavender oil.

Another embodiment comprises cyclosporin A, Tetronic 1107®, and Lavender oil.

Another embodiment comprises cyclosporin A, Tetronic 1307®, and Lavender oil.

Another embodiment comprises cyclosporin A, Tetronic 150R1®, and Lavender oil.

Another embodiment comprises cyclosporin A, Tetronic 304®, and Lavender oil.

Another embodiment comprises cyclosporin A, Tetronic 701®, and Lavender oil.

Another embodiment comprises cyclosporin A, Tetronic 901®, and Lavender oil.

Another embodiment comprises cyclosporin A, Tetronic 904®, and Lavender oil.

Another embodiment comprises cyclosporin A, Tetronic 908®, and Lavender oil.

Another embodiment comprises cyclosporin A, Tetronic 90R4®, and Lavender oil.

Another embodiment comprises cyclosporin A, Tomadol 1-3®, and Lavender oil.

Another embodiment comprises cyclosporin A, Tomadol 1-5®, and Lavender oil.

Another embodiment comprises cyclosporin A, Tomadol 1-7®, and Lavender oil.

Another embodiment comprises cyclosporin A, Tomadol 23-1®, and Lavender oil.

Another embodiment comprises cyclosporin A, Tomadol 23-3®, and Lavender oil.

Another embodiment comprises cyclosporin A, Tomadol 23-5®, and Lavender oil.

Another embodiment comprises cyclosporin A, Tomadol 23-6.5®, and Lavender oil.

Another embodiment comprises cyclosporin A, Tomadol 25-12®, and Lavender oil.

Another embodiment comprises cyclosporin A, Tomadol 25-3®, and Lavender oil.

Another embodiment comprises cyclosporin A, Tomadol 25-7®, and Lavender oil.

Another embodiment comprises cyclosporin A, Tomadol 25-9®, and Lavender oil.

Another embodiment comprises cyclosporin A, Tomadol 45-13®, and Lavender oil.

Another embodiment comprises cyclosporin A, Tomadol 45-2.5®, and Lavender oil.

Another embodiment comprises cyclosporin A, Tomadol 45-7®, and Lavender oil.

Another embodiment comprises cyclosporin A, Tomadol 91-2.5®, and Lavender oil.

Another embodiment comprises cyclosporin A, Tomadol 91-6®, and Lavender oil.

Another embodiment comprises cyclosporin A, Tomadol 91-8®, and Lavender oil.

Another embodiment comprises cyclosporin A, Triton X-100®, and Lavender oil.

Another embodiment comprises cyclosporin A, Triton X-102®, and Lavender oil.

Another embodiment comprises cyclosporin A, Triton X-114®, and Lavender oil.

Another embodiment comprises cyclosporin A, Triton X-15®, and Lavender oil.

Another embodiment comprises cyclosporin A, Triton X-165 (70%)®, and Lavender oil.

Another embodiment comprises cyclosporin A, Triton X-207®, and Lavender oil.

Another embodiment comprises cyclosporin A, Triton X-305 (70%)®, and Lavender oil.

Another embodiment comprises cyclosporin A, Triton X-35®, and Lavender oil.

Another embodiment comprises cyclosporin A, Triton X-405 (70%)®, and Lavender oil.

Another embodiment comprises cyclosporin A, Triton X-45®, and Lavender oil.

Another embodiment comprises cyclosporin A, Triton X-705 (70%)®, and Lavender oil.

Another embodiment comprises cyclosporin A, Volpo S-10®, and Lavender oil.

Another embodiment comprises cyclosporin A, Volpo S-2®, and Lavender oil.

Another embodiment comprises cyclosporin A, Volpo S-20®, and Lavender oil.

Another embodiment comprises cyclosporin A, Volpo-10®, and Lavender oil.

Another embodiment comprises cyclosporin A, Volpo-20®, and Lavender oil.

Another embodiment comprises cyclosporin A, Volpo-3®, and Lavender oil.

Another embodiment comprises cyclosporin A, Volpo-5®, and Lavender oil.

Another embodiment comprises cyclosporin A, Abex 2515®, and Citronella oil.

Another embodiment comprises cyclosporin A, Abex 2525/40®, and Citronella oil.

Another embodiment comprises cyclosporin A, Abex 2535®, and Citronella oil.

Another embodiment comprises cyclosporin A, Abex 2545®, and Citronella oil.

Another embodiment comprises cyclosporin A, Acconon E®, and Citronella oil.

Another embodiment comprises cyclosporin A, Alfonic 1216CO-9®, and Citronella oil.

Another embodiment comprises cyclosporin A, Alfonic 1412-3®, and Citronella oil.

Another embodiment comprises cyclosporin A, Alfonic TDA-12®, and Citronella oil.

Another embodiment comprises cyclosporin A, Alfonic TDA-3®, and Citronella oil.

Another embodiment comprises cyclosporin A, Alfonic TDA-4®, and Citronella oil.

Another embodiment comprises cyclosporin A, Alfonic TDA-6®, and Citronella oil.

Another embodiment comprises cyclosporin A, Alfonic TDA-7®, arid Citronella oil.

Another embodiment comprises cyclosporin A, Alfonic TDA-8®, and Citronella oil.

Another embodiment comprises cyclosporin A, Alfonic TDA-9®, and Citronella oil.

Another embodiment comprises cyclosporin A, Alkamuls 400-DO®, and Citronella oil.

Another embodiment comprises cyclosporin A, Alkamuls 400-MO/E®, and Citronella oil.

Another embodiment comprises cyclosporin A, Ameroxol OE-10®, and Citronella oil.

Another embodiment comprises cyclosporin A, Ameroxol OE-2®, and Citronella oil.

Another embodiment comprises cyclosporin A, Ameroxol OE-20®, and Citronella oil.

Another embodiment comprises cyclosporin A, Armix 176®, and Citronella oil.

Another embodiment comprises cyclosporin A, Armix 180-C®, and Citronella oil.

Another embodiment comprises cyclosporin A, Armix 183®, and Citronella oil.

Another embodiment comprises cyclosporin A, Brij 35®, and Citronella oil.

Another embodiment comprises cyclosporin A, Brij 35 Liquid®, and Citronella oil.

Another embodiment comprises cyclosporin A, Brij 35 SP®, and Citronella oil.

Another embodiment comprises cyclosporin A, Canasol MJ 2109®, and Citronella oil.

Another embodiment comprises cyclosporin A, Canasol MJ 52®, and Citronella oil.

Another embodiment comprises cyclosporin A, Canasol R 3600®, and Citronella oil.

Another embodiment comprises cyclosporin A, Canasol R 3603®, and Citronella oil.

Another embodiment comprises cyclosporin A, Canasol R 4000 H®, and Citronella oil.

Another embodiment comprises cyclosporin A, Cedepal CA-210®, and Citronella oil.

Another embodiment comprises cyclosporin A, Cedepal CA-520®, and Citronella oil.

Another embodiment comprises cyclosporin A, Cedepal CA-630®, and Citronella oil.

Another embodiment comprises cyclosporin A, Cedepal CA-720®, and Citronella oil.

Another embodiment comprises cyclosporin A, Cedepal CA-890®, and Citronella oil.

Another embodiment comprises cyclosporin A, Cedepal CA-897®, and Citronella oil.

Another embodiment comprises cyclosporin A, Cedepal CO-210®, and Citronella oil.

Another embodiment comprises cyclosporin A, Cedepal CO-430®, and Citronella oil.

Another embodiment comprises cyclosporin A, Cedepal CO-530®, and Citronella oil.

Another embodiment comprises cyclosporin A, Cedepal CO-630®, and Citronella oil.

Another embodiment comprises cyclosporin A, Cedepal CO-710®, and Citronella oil.

Another embodiment comprises cyclosporin A, Cedepal CO-730®, and Citronella oil.

Another embodiment comprises cyclosporin A, Cedepal CO-880®, and Citronella oil.

Another embodiment comprises cyclosporin A, Cedepal CO-887®, and Citronella oil.

Another embodiment comprises cyclosporin A, Cedepal CO-890®, and Citronella oil.

Another embodiment comprises cyclosporin A, Cedepal CO-897®, and Citronella oil.

Another embodiment comprises cyclosporin A, Cedepal CO-970®, and Citronella oil.

Another embodiment comprises cyclosporin A, Cedepal CO-977®, and Citronella oil.

Another embodiment comprises cyclosporin A, Cedepal CO-990®, and Citronella oil.

Another embodiment comprises cyclosporin A, Cedepal CO-997®, and Citronella oil.

Another embodiment comprises cyclosporin A, Chemal BP 261®, and Citronella oil.

Another embodiment comprises cyclosporin A, Chemal BP 262®, and Citronella oil.

Another embodiment comprises cyclosporin A, Chemal BP 3172®, and Citronella oil.

Another embodiment comprises cyclosporin A, Chemal BP 3174®, and Citronella oil.

Another embodiment comprises cyclosporin A, Chemal DA-4®, and Citronella oil.

Another embodiment comprises cyclosporin A, Chemal DA-6®, and Citronella oil.

Another embodiment comprises cyclosporin A, Chemal DA-9®, and Citronella oil.

Another embodiment comprises cyclosporin A, Chemal LA-23®, and Citronella oil.

Another embodiment comprises cyclosporin A, Chemal LA-4®, and Citronella oil.

Another embodiment comprises cyclosporin A, Chemal LA-9®, and Citronella oil.

Another embodiment comprises cyclosporin A, Chemal OA-20G®, and Citronella oil.

Another embodiment comprises cyclosporin A, Chemal OA-5®, and Citronella oil.

Another embodiment comprises cyclosporin A, Chemal OA-9®, and Citronella oil.

Another embodiment comprises cyclosporin A, Chemal TDA-12®, and Citronella oil.

Another embodiment comprises cyclosporin A, Chemal TDA-15®, and Citronella oil.

Another embodiment comprises cyclosporin A, Chemal TDA-3®, and Citronella oil.

Another embodiment comprises cyclosporin A, Chemal TDA-6®, and Citronella oil.

Another embodiment comprises cyclosporin A, Chemal TDA-9®, and Citronella oil.

Another embodiment comprises cyclosporin A, Chemax CO-16®, and Citronella oil.

Another embodiment comprises cyclosporin A, Chemax CO-200/50®, and Citronella oil.

Another embodiment comprises cyclosporin A, Chemax CO-25®, and Citronella oil.

Another embodiment comprises cyclosporin A, Chemax CO-30®, and Citronella oil.

Another embodiment comprises cyclosporin A, Chemax CO-36®, and Citronella oil.

Another embodiment comprises cyclosporin A, Chemax CO-40®, and Citronella oil.

Another embodiment comprises cyclosporin A, Chemax CO-5®, and Citronella oil.

Another embodiment comprises cyclosporin A, Chemax CO-80®, and Citronella oil.

Another embodiment comprises cyclosporin A, Chemax DNP-150/50®, and Citronella oil.

Another embodiment comprises cyclosporin A, Chemax DNP-18®, and Citronella oil.

Another embodiment comprises cyclosporin A, Chemax DNP-8®, and Citronella oil.

Another embodiment comprises cyclosporin A, Chemax E 200 ML®, and Citronella oil.

Another embodiment comprises cyclosporin A, Chemax E 200 MO®, and Citronella oil.

Another embodiment comprises cyclosporin A, Chemax E 400 ML®, and Citronella oil.

Another embodiment comprises cyclosporin A, Chemax E 400 MO®, and Citronella oil.

Another embodiment comprises cyclosporin A, Chemax E 600 ML®, and Citronella oil.

Another embodiment comprises cyclosporin A, Chemax E 600 MO®, and Citronella oil.

Another embodiment comprises cyclosporin A, Chemax E 1000 MO®, and Citronella oil.

Another embodiment comprises cyclosporin A, Chemax E 1000 MS®, and Citronella oil.

Another embodiment comprises cyclosporin A, Chemax E200 MS®, and Citronella oil.

Another embodiment comprises cyclosporin A, Chemax E400 MS®, and Citronella oil.

Another embodiment comprises cyclosporin A, Chemax E600 MS®, and Citronella oil.

Another embodiment comprises cyclosporin A, Chemax HCO-16®, and Citronella oil.

Another embodiment comprises cyclosporin A, Chemax HCO-200/50®, and Citronella oil.

Another embodiment comprises cyclosporin A, Chemax HCO-25®, and Citronella oil.

Another embodiment comprises cyclosporin A, Chemax HCO-5®, and Citronella oil.

Another embodiment comprises cyclosporin A, Chemax TO-16®, and Citronella oil.

Another embodiment comprises cyclosporin A, Chemax TO-8®, and Citronella oil.

Another embodiment comprises cyclosporin A, Colalube 3409®, and Citronella oil.

Another embodiment comprises cyclosporin A, Colalube 3411®, and Citronella oil.

Another embodiment comprises cyclosporin A, Colalube 3413®, and Citronella oil.

Another embodiment comprises cyclosporin A, Colalube 3414®, and Citronella oil.

Another embodiment comprises cyclosporin A, Colalube 3415®, and Citronella oil.

Another embodiment comprises cyclosporin A, Colalube 3416®, and Citronella oil.

Another embodiment comprises cyclosporin A, Colalube 3417®, and Citronella oil.

Another embodiment comprises cyclosporin A, Delonic NPE-100®, and Citronella oil.

Another embodiment comprises cyclosporin A, Delonic NPE-30®, and Citronella oil.

Another embodiment comprises cyclosporin A, Delonic NPE-40®, and Citronella oil.

Another embodiment comprises cyclosporin A, Delonic OPE-10®, and Citronella oil.

Another embodiment comprises cyclosporin A, Delonic OPE-12®, and Citronella oil.

Another embodiment comprises cyclosporin A, Delonic OPE-30®, and Citronella oil.

Another embodiment comprises cyclosporin A, Delonic OPE-40®, and Citronella oil.

Another embodiment comprises cyclosporin A, Delonic OPE-5®, and Citronella oil.

Another embodiment comprises cyclosporin A, Delonic OPE-7.5®, and Citronella oil.

Another embodiment comprises cyclosporin A, DePeg 16-CO®, and Citronella oil.

Another embodiment comprises cyclosporin A, DePeg 25-CO®, and Citronella oil.

Another embodiment comprises cyclosporin A, DePeg 30-CO®, and Citronella oil.

Another embodiment comprises cyclosporin A, DePeg 40-CO®, and Citronella oil.

Another embodiment comprises cyclosporin A, DePeg 5-CO®, and Citronella oil.

Another embodiment comprises cyclosporin A, DePeg 80-CO®, and Citronella oil.

Another embodiment comprises cyclosporin A, DeSonic S-100®, and Citronella oil.

Another embodiment comprises cyclosporin A, DeSonic S-114®, and Citronella oil.

Another embodiment comprises cyclosporin A, DeSonic S-405®, and Citronella oil.

Another embodiment comprises cyclosporin A, DeSonic S-45®, and Citronella oil.

Another embodiment comprises cyclosporin A, DeSonic TDA-9®, and Citronella oil.

Another embodiment comprises cyclosporin A, DeThox Acid O-14®, and Citronella oil.

Another embodiment comprises cyclosporin A, DeThox Acid O-16®, and Citronella oil.

Another embodiment comprises cyclosporin A, DeThox Acid O-9®, and Citronella oil.

Another embodiment comprises cyclosporin A, DeThox Acid TO-14®, and Citronella oil.

Another embodiment comprises cyclosporin A, DeThox Acid TO-16.5®, and Citronella oil.

Another embodiment comprises cyclosporin A, DeThox Acid TO-8.5®, and Citronella oil.

Another embodiment comprises cyclosporin A, Dur-Em 114®, and Citronella oil.

Another embodiment comprises cyclosporin A, Dur-Em 117®, and Citronella oil.

Another embodiment comprises cyclosporin A, Dur-Em 204®, and Citronella oil.

Another embodiment comprises cyclosporin A, Dur-Em 207®, and Citronella oil.

Another embodiment comprises cyclosporin A, Dur-Em 207E®, and Citronella oil.

Another embodiment comprises cyclosporin A, Emerest 2704®, and Citronella oil.

Another embodiment comprises cyclosporin A, Emerest 2712®, and Citronella oil.

Another embodiment comprises cyclosporin A, Emerest 2715®, and Citronella oil.

Another embodiment comprises cyclosporin A, Ethal EH-2®, and Citronella oil.

Another embodiment comprises cyclosporin A, Ethal EH-5®, and Citronella oil.

Another embodiment comprises cyclosporin A, Ethal OA-23®, and Citronella oil.

Another embodiment comprises cyclosporin A, Ethosperse CA-20®, and Citronella oil.

Another embodiment comprises cyclosporin A, Ethosperse LA-12®, and Citronella oil.

Another embodiment comprises cyclosporin A, Ethosperse LA-23®, and Citronella oil.

Another embodiment comprises cyclosporin A, Ethosperse LA-4®, and Citronella oil.

Another embodiment comprises cyclosporin A, Ethox 3095®, and Citronella oil.

Another embodiment comprises cyclosporin A, Ethox CO-16®, and Citronella oil.

Another embodiment comprises cyclosporin A, Ethox CO-200®, and Citronella oil.

Another embodiment comprises cyclosporin A, Ethox CO-25®, and Citronella oil.

Another embodiment comprises cyclosporin A, Ethox CO-30®, and Citronella oil.

Another embodiment comprises cyclosporin A, Ethox CO-36®, and Citronella oil.

Another embodiment comprises cyclosporin A, Ethox CO-40®, and Citronella oil.

Another embodiment comprises cyclosporin A, Ethox CO-5®, and Citronella oil.

Another embodiment comprises cyclosporin A, Ethox DO-14®, and Citronella oil.

Another embodiment comprises cyclosporin A, Ethox DO-9®, and Citronella oil.

Another embodiment comprises cyclosporin A, Ethox MA-8®, and Citronella oil.

Another embodiment comprises cyclosporin A, Ethox MI-14®, and Citronella oil.

Another embodiment comprises cyclosporin A, Ethox ML-14®, and Citronella oil.

Another embodiment comprises cyclosporin A, Ethox ML-5®, and Citronella oil.

Another embodiment comprises cyclosporin A, Ethox MO-14®, and Citronella oil.

Another embodiment comprises cyclosporin A, Ethox MO-9®, and Citronella oil.

Another embodiment comprises cyclosporin A, Ethox MS-14®, and Citronella oil.

Another embodiment comprises cyclosporin A, Ethox MS-23®, and Citronella oil.

Another embodiment comprises cyclosporin A, Ethox MS-40®, and Citronella oil.

Another embodiment comprises cyclosporin A, Ethox MS-8®, and Citronella oil.

Another embodiment comprises cyclosporin A, Ethox TO-16®, and Citronella oil.

Another embodiment comprises cyclosporin A, Ethox TO-8®, and Citronella oil.

Another embodiment comprises cyclosporin A, Ethylan SN-120®, and Citronella oil.

Another embodiment comprises cyclosporin A, Ethylan SN-70®, and Citronella oil.

Another embodiment comprises cyclosporin A, Ethylan SN-90®, and Citronella oil.

Another embodiment comprises cyclosporin A, Flexricin 13®, and Citronella oil.

Another embodiment comprises cyclosporin A, Flexricin 15®, and Citronella oil.

Another embodiment comprises cyclosporin A, Genapol 1261®, and Citronella oil.

Another embodiment comprises cyclosporin A, Generol 122 E25®, and Citronella oil.

Another embodiment comprises cyclosporin A, Glicepol 160®, and Citronella oil.

Another embodiment comprises cyclosporin A, Glicepol 560®, and Citronella oil.

Another embodiment comprises cyclosporin A, Glicepol GMS 20®, and Citronella oil.

Another embodiment comprises cyclosporin A, Hetoxide BN-13®, and Citronella oil.

Another embodiment comprises cyclosporin A, Hetoxide DNP-4®, and Citronella oil.

Another embodiment comprises cyclosporin A, Hetoxide DNP-9.6®, and Citronella oil.

Another embodiment comprises cyclosporin A, Hetoxide G-26®, and Citronella oil.

Another embodiment comprises cyclosporin A, Hetoxide G-7®, and Citronella oil.

Another embodiment comprises cyclosporin A, Hetoxide GT-20®, and Citronella oil.

Another embodiment comprises cyclosporin A, Hetoxide HC-40®, and Citronella oil.

Another embodiment comprises cyclosporin A, Hetoxol C-24®, and Citronella oil.

Another embodiment comprises cyclosporin A, Hetoxol CA-10®, and Citronella oil.

Another embodiment comprises cyclosporin A, Hetoxol CA-2®, and Citronella oil.

Another embodiment comprises cyclosporin A, Hetoxol CA-20®, and Citronella oil.

Another embodiment comprises cyclosporin A, Hetoxol CAWS®, and Citronella oil.

Another embodiment comprises cyclosporin A, Hetoxol CD-3®, and Citronella oil.

Another embodiment comprises cyclosporin A, Hetoxol CD-4®, and Citronella oil.

Another embodiment comprises cyclosporin A, Hetoxol CS-15®, and Citronella oil.

Another embodiment comprises cyclosporin A, Hetoxol CS-20®, and Citronella oil.

Another embodiment comprises cyclosporin A, Hetoxol CS-20D®, and Citronella oil.

Another embodiment comprises cyclosporin A, Hetoxol CS-25®, and Citronella oil.

Another embodiment comprises cyclosporin A, Hetoxol CS-50®, and Citronella oil.

Another embodiment comprises cyclosporin A, Hetoxoi CS-9®, and Citronella oil.

Another embodiment comprises cyclosporin A, Hetoxol CSA-15®, and Citronella oil.

Another embodiment comprises cyclosporin A, Hetoxol D®, and Citronella oil.

Another embodiment comprises cyclosporin A, Hetoxol G®, and Citronella oil.

Another embodiment comprises cyclosporin A, Hetoxol J®, and Citronella oil.

Another embodiment comprises cyclosporin A, Hetoxol L®, and Citronella oil.

Another embodiment comprises cyclosporin A, Hetoxol L-23®, and Citronella oil.

Another embodiment comprises cyclosporin A, Hetoxol L-4®, and Citronella oil.

Another embodiment comprises cyclosporin A, Hetoxol L-9®, and Citronella oil.

Another embodiment comprises cyclosporin A, Hetoxol LS-9®, and Citronella oil.

Another embodiment comprises cyclosporin A, Hetoxol M-3®, and Citronella oil.

Another embodiment comprises cyclosporin A, Hetoxol OA-10 Special®, and Citronella oil.

Another embodiment comprises cyclosporin A, Hetoxol OA-20 Special®, and Citronella oil.

Another embodiment comprises cyclosporin A, Hetoxol OA-3 Special®, and Citronella oil.

Another embodiment comprises cyclosporin A, Hetoxol OA-5 Special®, and Citronella oil.

Another embodiment comprises cyclosporin A, Hetoxol OL-10®, and Citronella oil.

Another embodiment comprises cyclosporin A, Hetoxol OL-2®, and Citronella oil.

Another embodiment comprises cyclosporin A, Hetoxol OL-23®, and Citronella oil.

Another embodiment comprises cyclosporin A, Hetoxol OL-4®, and Citronella oil.

Another embodiment comprises cyclosporin A, Hetoxol OL-40®, and Citronella oil.

Another embodiment comprises cyclosporin A, Hetoxol OL-5®, and Citronella oil.

Another embodiment comprises cyclosporin A, Hetoxol PLA®, and Citronella oil.

Another embodiment comprises cyclosporin A, Hetoxol SP-15®, and Citronella oil.

Another embodiment comprises cyclosporin A, Hetoxol STA-10®, and Citronella oil.

Another embodiment comprises cyclosporin A, Hetoxol STA-2®, and Citronella oil.

Another embodiment comprises cyclosporin A, Hetoxol STA-30®, and Citronella oil.

Another embodiment comprises cyclosporin A, Hetoxol TD-12®, and Citronella oil.

Another embodiment comprises cyclosporin A, Hetoxol TD-18®, and Citronella oil.

Another embodiment comprises cyclosporin A, Hetoxol TD-3®, and Citronella oil.

Another embodiment comprises cyclosporin A, Hetoxol TD-6®, and Citronella oil.

Another embodiment comprises cyclosporin A, Hetoxol TD-9®, and Citronella oil.

Another embodiment comprises cyclosporin A, Hetoxol TDEP-15®, and Citronella oil.

Another embodiment comprises cyclosporin A, Hetoxol TDEP-63®, and Citronella oil.

Another embodiment comprises cyclosporin A, Iconol 24-12®, and Citronella oil.

Another embodiment comprises cyclosporin A, Iconol 24-23®, and Citronella oil.

Another embodiment comprises cyclosporin A, Iconol 24-4®, and Citronella oil.

Another embodiment comprises cyclosporin A, Iconol DA-4®, and Citronella oil.

Another embodiment comprises cyclosporin A, Iconol DA-6®, and Citronella oil.

Another embodiment comprises cyclosporin A, Iconol TDA-10®, and Citronella oil.

Another embodiment comprises cyclosporin A, Iconol TDA-3®, and Citronella oil.

Another embodiment comprises cyclosporin A, Iconol TDA-6®, and Citronella oil.

Another embodiment comprises cyclosporin A, Iconol TDA-8-90%®, and Citronella oil.

Another embodiment comprises cyclosporin A, Iconol TDA-9®, and Citronella oil.

Another embodiment comprises cyclosporin A, Igepal CA-210®, and Citronella oil.

Another embodiment comprises cyclosporin A, Igepal CA-407®, and Citronella oil.

Another embodiment comprises cyclosporin A, Igepal CA-420®, and Citronella oil.

Another embodiment comprises cyclosporin A, Igepal CA-520®, and Citronella oil.

Another embodiment comprises cyclosporin A, Igepal CA-620®, and Citronella oil.

Another embodiment comprises cyclosporin A, Igepal CA-630®, and Citronella oil.

Another embodiment comprises cyclosporin A, Igepal CA-720®, and Citronella oil.

Another embodiment comprises cyclosporin A, Igepal CA-877®, and Citronelia oil.

Another embodiment comprises cyclosporin A, Igepal CA-887®, and Citronella oil.

Another embodiment comprises cyclosporin A, Igepal CA-897®, and Citronella oil.

Another embodiment comprises cyclosporin A, Igepal CO-210®, and Citronella oil.

Another embodiment comprises cyclosporin A, Igepal CO-430®, and Citronella oil.

Another embodiment comprises cyclosporin A, Igepal CO-520®, and Citronella oil.

Another embodiment comprises cyclosporin A, Igepal CO-530®, and Citronella oil.

Another embodiment comprises cyclosporin A, Igepal CO-610®, and Citronella oil.

Another embodiment comprises cyclosporin A, Igepal CO-630®, and Citronella oil.

Another embodiment comprises cyclosporin A, Igepal CO-630 Special®, and Citronella oil.

Another embodiment comprises cyclosporin A, Igepal CO-660®, and Citronella oil.

Another embodiment comprises cyclosporin A, Igepal CO-710®, and Citronella oil.

Another embodiment comprises cyclosporin A, Igepal CO-720®, and Citronella oil.

Another embodiment comprises cyclosporin A, Igepal CO-730®, and Citronella oil.

Another embodiment comprises cyclosporin A, Igepal CO-738®, and Citronella oil.

Another embodiment comprises cyclosporin A, Igepal CO-850®, and Citronella oil.

Another embodiment comprises cyclosporin A, Igepal CO-880®, and Citronella oil.

Another embodiment comprises cyclosporin A, Igepal CO-887®, and Citronella oil.

Another embodiment comprises cyclosporin A, Igepal CO-890®, and Citronella oil.

Another embodiment comprises cyclosporin A, Igepal CO-897®, and Citronella oil.

Another embodiment comprises cyclosporin A, Igepal CO-970®, and Citronella oil.

Another embodiment comprises cyclosporin A, Igepal CO-977®, and Citronella oil.

Another embodiment comprises cyclosporin A, Igepal CO-987®, and Citronella oil.

Another embodiment comprises cyclosporin A, Igepal CO-990 FLK®, and Citronella oil.

Another embodiment comprises cyclosporin A, Igepal CO-997®, and Citronella oil.

Another embodiment comprises cyclosporin A, Igepal DM-430®, and Citronella oil.

Another embodiment comprises cyclosporin A, Igepal DM-530®, and Citronella oil.

Another embodiment comprises cyclosporin A, Igepal DM-710®, and Citronella oil.

Another embodiment comprises cyclosporin A, Igepal DM-730®, and Citronella oil.

Another embodiment comprises cyclosporin A, Igepal DM-880®, and Citronella oil.

Another embodiment comprises cyclosporin A, Igepal DM-970 FLK®, and Citronella oil.

Another embodiment comprises cyclosporin A, Igepal OD-410®, and Citronella oil.

Another embodiment comprises cyclosporin A, Igepal RC-520®, and Citronella oil.

Another embodiment comprises cyclosporin A, Igepal RC-620®, and Citronella oil.

Another embodiment comprises cyclosporin A, Igepal RC-630®, and Citronella oil.

Another embodiment comprises cyclosporin A, Lexemul CS-20®, and Citronella oil.

Another embodiment comprises cyclosporin A, Lipo GMS 450®, and Citronella oil.

Another embodiment comprises cyclosporin A, Lipo Polyglycol 1000®, and Citronella oil.

Another embodiment comprises cyclosporin A, Lipo Polyglycol 200®, and Citronella oil.

Another embodiment comprises cyclosporin A, Lipo Polyglycol 300®, and Citronella oil.

Another embodiment comprises cyclosporin A, Lipo Polyglycol 3350®, and Citronella oil.

Another embodiment comprises cyclosporin A, Lipo Polyglycol 400®, and Citronella oil.

Another embodiment comprises cyclosporin A, Lipo Polyglycol 600®, and Citronella oil.

Another embodiment comprises cyclosporin A, Lipocol C-10®, and Citronella oil.

Another embodiment comprises cyclosporin A, Lipocol C-2®, and Citronella oil.

Another embodiment comprises cyclosporin A, Lipocol C-20®, and Citronella oil.

Another embodiment comprises cyclosporin A, Lipocol L-12®, and Citronella oil.

Another embodiment comprises cyclosporin A, Lipocol L-23®, and Citronella oil.

Another embodiment comprises cyclosporin A, Lipocol L-4®, and Citronella oil.

Another embodiment comprises cyclosporin A, Lipocol O-10®, and Citronella oil.

Another embodiment comprises cyclosporin A, Lipocol O-2®, and Citronella oil.

Another embodiment comprises cyclosporin A, Lipocol O-20®, and Citronella oil.

Another embodiment comprises cyclosporin A, Lipocol O-5®, and Citronella oil.

Another embodiment comprises cyclosporin A, Lipocol S-10®, and Citronella oil.

Another embodiment comprises cyclosporin A, Lipocol S-2®, and Citronella oil.

Another embodiment comprises cyclosporin A, Lipocol S-20®, and Citronella oil.

Another embodiment comprises cyclosporin A, Lipocol SC-15®, and Citronella oil.

Another embodiment comprises cyclosporin A, Lipocol SC-20®, and Citronella oil.

Another embodiment comprises cyclosporin A, Lipocol SC-4®, and Citronella oil.

Another embodiment comprises cyclosporin A, Lipocol TD-12®, and Citronella oil.

Another embodiment comprises cyclosporin A, Lumulse 40-O K®, and Citronella oil.

Another embodiment comprises cyclosporin A, Lumulse 42-O K®, and Citronella oil.

Another embodiment comprises cyclosporin A, Lumulse GMT-40®, and Citronella oil.

Another embodiment comprises cyclosporin A, Lumulse PEG 1450®, and Citronella oil.

Another embodiment comprises cyclosporin A, Lumulse PEG 1450 NF®, and Citronella oil.

Another embodiment comprises cyclosporin A, Lumulse PEG 8000®, and Citronella oil.

Another embodiment comprises cyclosporin A, Lumulse PGO®, and Citronella oil.

Another embodiment comprises cyclosporin A, Lumulse POE (2) Oleyl Amine®, and Citronella oil.

Another embodiment comprises cyclosporin A, Magrabar PGO-104®, and Citronella oil.

Another embodiment comprises cyclosporin A, Mapeg 200 ML®, and Citronella oil.

Another embodiment comprises cyclosporin A, Mapeg 400 MO®, and Citronella oil.

Another embodiment comprises cyclosporin A, Mapeg 400 MOT®, and Citronella oil.

Another embodiment comprises cyclosporin A, Mapeg 400DO®, and Citronella oil.

Another embodiment comprises cyclosporin A, Mapeg 400DOT®, and Citronella oil.

Another embodiment comprises cyclosporin A, Mapeg 600DOT®, and Citronella oil.

Another embodiment comprises cyclosporin A, Marlox B 24/80®, and Citronella oil.

Another embodiment comprises cyclosporin A, Marlox FK 86®, and Citronella oil.

Another embodiment comprises cyclosporin A, Marlox MO 154®, and Citronella oil.

Another embodiment comprises cyclosporin A, Mazox CDA CG®, and Citronella oil.

Another embodiment comprises cyclosporin A, Monoglycerides Glyceryl Monestearate®, and Citronella oil.

Another embodiment comprises cyclosporin A, Myrj 45®, and Citronella oil.

Another embodiment comprises cyclosporin A, Myrj 52®, and Citronella oil.

Another embodiment comprises cyclosporin A, Myrj 52S®, and Citronella oil.

Another embodiment comprises cyclosporin A, Myrj 53®, and Citronella oil.

Another embodiment comprises cyclosporin A, Myrj 59®, and Citronella oil.

Another embodiment comprises cyclosporin A, Norfox NP-12®, and Citronella oil.

Another embodiment comprises cyclosporin A, Norfox NP-4®, and Citronella oil.

Another embodiment comprises cyclosporin A, Norfox NP-6®, and Citronella oil.

Another embodiment comprises cyclosporin A, Norfox NP-887®, and Citronella oil.

Another embodiment comprises cyclosporin A, Norfox NP-9®, and Citronella oil.

Another embodiment comprises cyclosporin A, Norfox NP-977®, and Citronella oil.

Another embodiment comprises cyclosporin A, Norfox OP-100®, and Citronella oil.

Another embodiment comprises cyclosporin A, Norfox OP-102®, and Citronella oil.

Another embodiment comprises cyclosporin A, Norfox OP-114®, and Citronella oil.

Another embodiment comprises cyclosporin A, Norfox OP-45®, and Citronella oil.

Another embodiment comprises cyclosporin A, Pegosperse 100-L®, and Citronella oil.

Another embodiment comprises cyclosporin A, Pegosperse 100-O®, and Citronella oil.

Another embodiment comprises cyclosporin A, Pegosperse 100-S®, and Citronella oil.

Another embodiment comprises cyclosporin A, Pegosperse 1500-MS®, and Citronella oil.

Another embodiment comprises cyclosporin A, Pegosperse 1750-MS®, and Citronella oil.

Another embodiment comprises cyclosporin A, Pegosperse 200 DL®, and Citronella oil.

Another embodiment comprises cyclosporin A, Pegosperse 200-ML®, and Citronella oil.

Another embodiment comprises cyclosporin A, Pegosperse 400-DL®, and Citronella oil.

Another embodiment comprises cyclosporin A, Pegosperse 400-DO®, and Citronella oil.

Another embodiment comprises cyclosporin A, Pegosperse 400-DS®, and Citronella oil.

Another embodiment comprises cyclosporin A, Pegosperse 400-ML®, and Citronella oil.

Another embodiment comprises cyclosporin A, Pegosperse 400-MO®, and Citronella oil.

Another embodiment comprises cyclosporin A, Pegosperse 400-MOT®, and Citronella oil.

Another embodiment comprises cyclosporin A, Pegosperse 400-MS®, and Citronella oil.

Another embodiment comprises cyclosporin A, Pegosperse 50-DS®, and Citronella oil.

Another embodiment comprises cyclosporin A, Pegosperse 50-MS®, and Citronella oil.

Another embodiment comprises cyclosporin A, Pegosperse 600-ML®, and Citronella oil.

Another embodiment comprises cyclosporin A, Pegosperse 600-MS®, and Citronella oil.

Another embodiment comprises cyclosporin A, Plurafac A-38®, and Citronella oil.

Another embodiment comprises cyclosporin A, Plurafac B-25-5®, and Citronella oil.

Another embodiment comprises cyclosporin A, Plurafac B-26®, and Citronella oil.

Another embodiment comprises cyclosporin A, Plurafac D-25®, and Citronella oil.

Another embodiment comprises cyclosporin A, Plurafac LF 131®, and Citronella oil.

Another embodiment comprises cyclosporin A, Plurafac LF 4030®, and Citronella oil.

Another embodiment comprises cyclosporin A, Plurafac LF 7000®, and Citronella oil.

Another embodiment comprises cyclosporin A, Plurafac RA-20®, and Citronella oil.

Another embodiment comprises cyclosporin A, Plurafac RA-30®, and Citronella oil.

Another embodiment comprises cyclosporin A, Plurafac RA-40®, and Citronella oil.

Another embodiment comprises cyclosporin A, Pluronic 10R5®, and Citronella oil.

Another embodiment comprises cyclosporin A, Pluronic 17R2®, and Citronella oil.

Another embodiment comprises cyclosporin A, Pluronic 17R4®, and Citronella oil.

Another embodiment comprises cyclosporin A, Pluronic 25R2®, and Citronella oil.

Another embodiment comprises cyclosporin A, Pluronic 25R4®, and Citronella oil.

Another embodiment comprises cyclosporin A, Pluronic 31R1®, and Citronella oil.

Another embodiment comprises cyclosporin A, Pluronic F108®, and Citronella oil.

Another embodiment comprises cyclosporin A, Pluronic F127®, and Citronella oil.

Another embodiment comprises cyclosporin A, Pluronic F38®, and Citronella oil.

Another embodiment comprises cyclosporin A, Pluronic F68®, and Citronella oil.

Another embodiment comprises cyclosporin A, Pluronic F68LF®, and Citronella oil.

Another embodiment comprises cyclosporin A, Pluronic F77®, and Citronella oil.

Another embodiment comprises cyclosporin A, Pluronic F87®, and Citronella oil.

Another embodiment comprises cyclosporin A, Pluronic F88®, and Citronella oil.

Another embodiment comprises cyclosporin A, Pluronic F98®, and Citronella oil.

Another embodiment comprises cyclosporin A, Pluronic L10®, and Citronella oil.

Another embodiment comprises cyclosporin A, Pluronic L101®, and Citronella oil.

Another embodiment comprises cyclosporin A, Pluronic L121®, and Citronella oil.

Another embodiment comprises cyclosporin A, Pluronic L31®, and Citronella oil.

Another embodiment comprises cyclosporin A, Pluronic L35®, and Citronella oil.

Another embodiment comprises cyclosporin A, Pluronic L43®, and Citronella oil.

Another embodiment comprises cyclosporin A, Pluronic L44®, and Citronella oil.

Another embodiment comprises cyclosporin A, Pluronic L61®, and Citronella oil.

Another embodiment comprises cyclosporin A, Pluronic L62®, and Citronella oil.

Another embodiment comprises cyclosporin A, Pluronic L62D®, and Citronella oil.

Another embodiment comprises cyclosporin A, Pluronic L62LF®, and Citronella oil.

Another embodiment comprises cyclosporin A, Pluronic L64®, and Citronella oil.

Another embodiment comprises cyclosporin A, Pluronic L81®, and Citronella oil.

Another embodiment comprises cyclosporin A, Pluronic L92®, and Citronella oil.

Another embodiment comprises cyclosporin A, Pluronic P103®, and Citronella oil.

Another embodiment comprises cyclosporin A, Pluronic P104®, and Citronella oil.

Another embodiment comprises cyclosporin A, Pluronic P105®, and Citronella oil.

Another embodiment comprises cyclosporin A, Pluronic P123®, and Citronella oil.

Another embodiment comprises cyclosporin A, Pluronic P65®, and Citronella oil.

Another embodiment comprises cyclosporin A, Pluronic P84®, and Citronella oil.

Another embodiment comprises cyclosporin A, Pluronic P85®, and Citronella oil.

Another embodiment comprises cyclosporin A, Polyaldo 10-2-P®, and Citronella oil.

Another embodiment comprises cyclosporin A, Polyaldo DGDO®, and Citronella oil.

Another embodiment comprises cyclosporin A, Polyaldo HGDS®, and Citronella oil.

Another embodiment comprises cyclosporin A, Polyaldo TGMS®, and Citronella oil.

Another embodiment comprises cyclosporin A, Protegin V®, and Citronella oil.

Another embodiment comprises cyclosporin A, Protegin W®, and Citronella oil.

Another embodiment comprises cyclosporin A, Protegin WX®, and Citronella oil.

Another embodiment comprises cyclosporin A, Protegin XV®, and Citronella oil.

Another embodiment comprises cyclosporin A, Rhodasurf BC-420®, and Citronella oil.

Another embodiment comprises cyclosporin A, Rhodasurf BC-610®, and Citronella oil.

Another embodiment comprises cyclosporin A, Rhodasurf BC-630®, and Citronella oil.

Another embodiment comprises cyclosporin A, Rhodasurf BC-720®, and Citronella oil.

Another embodiment comprises cyclosporin A, Rhodasurf BC-840®, and Citronella oil.

Another embodiment comprises cyclosporin A, Rhodasurf L 1®, and Citronella oil.

Another embodiment comprises cyclosporin A, Rhodasurf L 12®, and Citronella oil.

Another embodiment comprises cyclosporin A, Rhodasurf L 2®, and Citronella oil.

Another embodiment comprises cyclosporin A, Rhodasurf L 20®, and Citronella oil.

Another embodiment comprises cyclosporin A, Rhodasurf L 3®, and Citronella oil.

Another embodiment comprises cyclosporin A, Rhodasurf L 30®, and Citronella oil.

Another embodiment comprises cyclosporin A, Rhodasurf L 4®, and Citronella oil.

Another embodiment comprises cyclosporin A, Rhodasurf L 50®, and Citronella oil.

Another embodiment comprises cyclosporin A, Rhodasurf L 7/90®, and Citronella oil.

Another embodiment comprises cyclosporin A, Rhodasurf LA-3®, and Citronella oil.

Another embodiment comprises cyclosporin A, Rhodasurf LA-7®, and Citronella oil.

Another embodiment comprises cyclosporin A, Rhodasurf LAN-23®, and Citronella oil.

Another embodiment comprises cyclosporin A, Rhodasurf ON-870®, and Citronella oil.

Another embodiment comprises cyclosporin A, Rhodasurf ON-877®, and Citronella oil.

Another embodiment comprises cyclosporin A, Rhodasurf TDA-8/5®, and Citronella oil.

Another embodiment comprises cyclosporin A, Ritapro 100®, and Citronella oil.

Another embodiment comprises cyclosporin A, Ritapro 165®, and Citronella oil.

Another embodiment comprises cyclosporin A, Ritapro 200®, and Citronella oil.

Another embodiment comprises cyclosporin A, Ritapro 300®, and Citronella oil.

Another embodiment comprises cyclosporin A, Ritoleth 10®, and Citronella oil.

Another embodiment comprises cyclosporin A, Ritoleth 2®, and Citronella oil.

Another embodiment comprises cyclosporin A, Ritoleth 20®, and Citronella oil.

Another embodiment comprises cyclosporin A, Ritoleth 5®, and Citronella oil.

Another embodiment comprises cyclosporin A, Santone 10-10-0®, and Citronella oil.

Another embodiment comprises cyclosporin A, Santone 3-1-S XTR®, and Citronella oil.

Another embodiment comprises cyclosporin A, Santone 3-1-SH®, and Citronella oil.

Another embodiment comprises cyclosporin A, Santone 8-1-O®, and Citronella oil.

Another embodiment comprises cyclosporin A, Solulan 16®, and Citronella oil.

Another embodiment comprises cyclosporin A, Solulan 75®, and Citronella oil.

Another embodiment comprises cyclosporin A, Solulan C-24®, and Citronella oil.

Another embodiment comprises cyclosporin A, Solulan L-575®, and Citronella oil.

Another embodiment comprises cyclosporin A, Standamox CAW®, and Citronella oil.

Another embodiment comprises cyclosporin A, Surfine AZI-A®, and Citronella oil.

Another embodiment comprises cyclosporin A, Surfonic CO-15®, and Citronella oil.

Another embodiment comprises cyclosporin A, Surfonic CO-25®, and Citronella oil.

Another embodiment comprises cyclosporin A, Surfonic CO-30®, and Citronella oil.

Another embodiment comprises cyclosporin A, Surfonic CO-36®, and Citronella oil.

Another embodiment comprises cyclosporin A, Surfonic CO-42®, and Citronella oil.

Another embodiment comprises cyclosporin A, Surfonic E400-MO®, and Citronella oil.

Another embodiment comprises cyclosporin A, Surfonic L12-3®, and Citronella oil.

Another embodiment comprises cyclosporin A, Surfonic L12-6®, and Citronella oil.

Another embodiment comprises cyclosporin A, Surfonic L12-8®, and Citronella oil.

Another embodiment comprises cyclosporin A, Surfonic L24-12®, and Citronella oil.

Another embodiment comprises cyclosporin A, Surfonic L24-2®, and Citronella oil.

Another embodiment comprises cyclosporin A, Surfonic L24-22®, and Citronella oil.

Another embodiment comprises cyclosporin A, Surfonic L24-3®, and Citronella oil.

Another embodiment comprises cyclosporin A, Surfonic L24-4®, and Citronella oil.

Another embodiment comprises cyclosporin A, Surfonic L24-5®, and Citronella oil.

Another embodiment comprises cyclosporin A, Surfonic L24-7®, and Citronella oil.

Another embodiment comprises cyclosporin A, Surfonic L24-7.1®, and Citronella oil.

Another embodiment comprises cyclosporin A, Surfonic L24-9®, and Citronella oil.

Another embodiment comprises cyclosporin A, Surfonic LF-17®, and Citronella oil.

Another embodiment comprises cyclosporin A, Surfonic LF-18®, and Citronella oil.

Another embodiment comprises cyclosporin A, Surfonic LF-37®, and Citronella oil.

Another embodiment comprises cyclosporin A, Surfonic LF-41®, and Citronella oil.

Another embodiment comprises cyclosporin A, Surfonic LF-42®, and Citronella oil.

Another embodiment comprises cyclosporin A, Surfonic LF-47®, and Citronella oil.

Another embodiment comprises cyclosporin A, Surfonic LF-68®, and Citronella oil.

Another embodiment comprises cyclosporin A, Surfonic N-10®, and Citronella oil.

Another embodiment comprises cyclosporin A, Surfonic N-100®, and Citronella oil.

Another embodiment comprises cyclosporin A, Surfonic N-102®, and Citronella oil.

Another embodiment comprises cyclosporin A, Surfonic N-120®, and Citronella oil.

Another embodiment comprises cyclosporin A, Surfonic N-150®, and Citronella oil.

Another embodiment comprises cyclosporin A, Surfonic N-200®, and Citronella oil.

Another embodiment comprises cyclosporin A, Surfonic N-300®, and Citronella oil.

Another embodiment comprises cyclosporin A, Surfonic N-31.5®, and Citronella oil.

Another embodiment comprises cyclosporin A, Surfonic N-40®, and Citronella oil.

Another embodiment comprises cyclosporin A, Surfonic N-400®, and Citronella oil.

Another embodiment comprises cyclosporin A, Surfonic N-60®, and Citronella oil.

Another embodiment comprises cyclosporin A, Surfonic N-85®, and Citronella oil.

Another embodiment comprises cyclosporin A, Surfonic N-95®, and Citronella oil.

Another embodiment comprises cyclosporin A, Surfonic NB-18®, and Citronella oil.

Another embodiment comprises cyclosporin A, Surfonic NB-23®, and Citronella oil.

Another embodiment comprises cyclosporin A, Surfonic NB-25®, and Citronella oil.

Another embodiment comprises cyclosporin A, Surfonic NB-307®, and Citronella oil.

Another embodiment comprises cyclosporin A, Surfonic NB-31®, and Citronella oil.

Another embodiment comprises cyclosporin A, Surfonic NB-41®, and Citronella oil.

Another embodiment comprises cyclosporin A, Surfonic NB-43®, and Citronella oil.

Another embodiment comprises cyclosporin A, Surfonic OP-100®, and Citronella oil.

Another embodiment comprises cyclosporin A, Surfonic OP-120®, and Citronella oil.

Another embodiment comprises cyclosporin A, Surfonic OP-15®, and Citronella oil.

Another embodiment comprises cyclosporin A, Surfonic OP-35®, and Citronella oil.

Another embodiment comprises cyclosporin A, Surfonic OP-70®, and Citronella oil.

Another embodiment comprises cyclosporin A, Surfonic P1®, and Citronella oil.

Another embodiment comprises cyclosporin A, Surfonic P3®, and Citronella oil.

Another embodiment comprises cyclosporin A, Surfonic P5®, and Citronella oil.

Another embodiment comprises cyclosporin A, Surfonic P6®, and Citronella oil.

Another embodiment comprises cyclosporin A, Surfonic POA-17R2®, and Citronella oil.

Another embodiment comprises cyclosporin A, Surfonic POA-25R2®, and Citronella oil.

Another embodiment comprises cyclosporin A, Surfonic TDA-11®, and Citronella oil.

Another embodiment comprises cyclosporin A, Surfonic TDA-3B®, and Citronella oil.

Another embodiment comprises cyclosporin A, Surfonic TDA-6®, and Citronella oil.

Another embodiment comprises cyclosporin A, Surfonic TDA-6/88®, and Citronella oil.

Another embodiment comprises cyclosporin A, Surfonic TDA-8®, and Citronella oil.

Another embodiment comprises cyclosporin A, Surfonic TDA-8/90®, and Citronella oil.

Another embodiment comprises cyclosporin A, Surfonic TDA-9®, and Citronella oil.

Another embodiment comprises cyclosporin A, Surfonic TX-CCN®, and Citronella oil.

Another embodiment comprises cyclosporin A, Tagat CH 40®, and Citronella oil.

Another embodiment comprises cyclosporin A, Tagat CH 60®, and Citronella oil.

Another embodiment comprises cyclosporin A, Tagat L 2®, and Citronella oil.

Another embodiment comprises cyclosporin A, Tagat S®, and Citronella oil.

Another embodiment comprises cyclosporin A, Tagat S 2®, and Citronella oil.

Another embodiment comprises cyclosporin A, Tally 100 Plus®, and Citronella oil.

Another embodiment comprises cyclosporin A, T-Det A106®, and Citronella oil.

Another embodiment comprises cyclosporin A, T-Det A109®, and Citronella oil.

Another embodiment comprises cyclosporin A, T-Det A136®, and Citronella oil.

Another embodiment comprises cyclosporin A, T-Det A139®, and Citronella oil.

Another embodiment comprises cyclosporin A, T-Det A2412®, and Citronella oil.

Another embodiment comprises cyclosporin A, T-Det A243®, and Citronella oil.

Another embodiment comprises cyclosporin A, T-Det A247®, and Citronella oil.

Another embodiment comprises cyclosporin A, T-Det A249®, and Citronella oil.

Another embodiment comprises cyclosporin A, T-Det A467®, and Citronella oil.

Another embodiment comprises cyclosporin A, T-Det COE®, and Citronelia oil.

Another embodiment comprises cyclosporin A, T-Det DD-10®, and Citronella oil.

Another embodiment comprises cyclosporin A, T-Det DD-5®, and Citronella oil.

Another embodiment comprises cyclosporin A, T-Det DD-7®, and Citronella oil.

Another embodiment comprises cyclosporin A, T-Det EPO-104®, and Citronella oil.

Another embodiment comprises cyclosporin A, T-Det EPO-61®, and Citronella oil.

Another embodiment comprises cyclosporin A, T-Det EPO-62®, and Citronella oil.

Another embodiment comprises cyclosporin A, T-Det EPO-64®, and Citronella oil.

Another embodiment comprises cyclosporin A, T-Det N-1.5®, and Citronella oil.

Another embodiment comprises cyclosporin A, T-Det N-10.5®, and Citronella oil.

Another embodiment comprises cyclosporin A, T-Det N-100®, and Citronella oil.

Another embodiment comprises cyclosporin A, T-Det N-1007®, and Citronella oil.

Another embodiment comprises cyclosporin A, T-Det N-12®, and Citronella oil.

Another embodiment comprises cyclosporin A, T-Det N-14®, and Citronella oil.

Another embodiment comprises cyclosporin A, T-Det N-20®, and Citronella oil.

Another embodiment comprises cyclosporin A, T-Det N-30®, and Citronella oil.

Another embodiment comprises cyclosporin A, T-Det N-307®, and Citronella oil.

Another embodiment comprises cyclosporin A, T-Det N-4®, and Citronella oil.

Another embodiment comprises cyclosporin A, T-Det N-40®, and Citronella oil.

Another embodiment comprises cyclosporin A, T-Det N-407®, and Citronella oil.

Another embodiment comprises cyclosporin A, T-Det N-50®, and Citronella oil.

Another embodiment comprises cyclosporin A, T-Det N-507®, and Citronella oil.

Another embodiment comprises cyclosporin A, T-Det N-6®, and Citronella oil.

Another embodiment comprises cyclosporin A, T-Det N-70®, and Citronella oil.

Another embodiment comprises cyclosporin A, T-Det N-8®, and Citronella oil.

Another embodiment comprises cyclosporin A, T-Det N-9.5®, and Citronella oil.

Another embodiment comprises cyclosporin A, T-Det O-12®, and Citronella oil.

Another embodiment comprises cyclosporin A, T-Det O-165®, and Citronella oil.

Another embodiment comprises cyclosporin A, T-Det O-307®, and Citronella oil.

Another embodiment comprises cyclosporin A, T-Det O-40®, and Citronella oil.

Another embodiment comprises cyclosporin A, T-Det O-407®, and Citronella oil.

Another embodiment comprises cyclosporin A, T-Det O-6®, and Citronella oil.

Another embodiment comprises cyclosporin A, T-Det O-8®, and Citronella oil.

Another embodiment comprises cyclosporin A, T-Det O-9®, and Citronella oil.

Another embodiment comprises cyclosporin A, Tegin®, and Citronella oil.

Another embodiment comprises cyclosporin A, Tergitol 15-S-12®, and Citronella oil.

Another embodiment comprises cyclosporin A, Tergitol 15-S-15®, and Citronella oil.

Another embodiment comprises cyclosporin A, Tergitol 15-S-20®, and Citronella oil.

Another embodiment comprises cyclosporin A, Tergitol 15-S-20 (80AQ)®, and Citronella oil.

Another embodiment comprises cyclosporin A, Tergitol 15-S-3®, and Citronella oil.

Another embodiment comprises cyclosporin A, Tergitol 15-S-30®, and Citronella oil.

Another embodiment comprises cyclosporin A, Tergitol 15-S-40®, and Citronella oil.

Another embodiment comprises cyclosporin A, Tergitol 15-S-40 (70AQ)®, and Citronella oil.

Another embodiment comprises cyclosporin A, Tergitol 15-S-5®, and Citronella oil.

Another embodiment comprises cyclosporin A, Tergitol 15-S-7®, and Citronella oil.

Another embodiment comprises cyclosporin A, Tergitol 15-S-9®, and Citronella oil.

Another embodiment comprises cyclosporin A, Tergitol L-101®, and Citronella oil.

Another embodiment comprises cyclosporin A, Tergitol L-61®, and Citronella oil.

Another embodiment comprises cyclosporin A, Tergitol L-62®, and Citronella oil.

Another embodiment comprises cyclosporin A, Tergitol L-64®, and Citronella oil.

Another embodiment comprises cyclosporin A, Tergitol L-81®, and Citronella oil.

Another embodiment comprises cyclosporin A, Tergitol NP-10®, and Citronella oil.

Another embodiment comprises cyclosporin A, Tergitol NP-11®, and Citronella oil.

Another embodiment comprises cyclosporin A, Tergitol NP-12®, and Citronella oil.

Another embodiment comprises cyclosporin A, Tergitol NP-13®, and Citronella oil.

Another embodiment comprises cyclosporin A, Tergitol NP-15®, and Citronella oil.

Another embodiment comprises cyclosporin A, Tergitol NP-30®, and Citronella oil.

Another embodiment comprises cyclosporin A, Tergitol NP-30 (70 AQ)®, and Citronella oil.

Another embodiment comprises cyclosporin A, Tergitol NP-4®, and Citronella oil.

Another embodiment comprises cyclosporin A, Tergitol NP-40®, and Citronella oil.

Another embodiment comprises cyclosporin A, Tergitol NP-40 (70 AQ)®, and Citronella oil.

Another embodiment comprises cyclosporin A, Tergitol NP-50 (70 AQ)®, and Citronella oil.

Another embodiment comprises cyclosporin A, Tergitol NP-55 (70 AQ)®, and Citronella oil.

Another embodiment comprises cyclosporin A, Tergitol NP-6®, and Citronella oil.

Another embodiment comprises cyclosporin A, Tergitol NP-7®, and Citronella oil.

Another embodiment comprises cyclosporin A, Tergitol NP-70 (70 AQ)®, and Citronella oil.

Another embodiment comprises cyclosporin A, Tergitol NP-8®, and Citronella oil.

Another embodiment comprises cyclosporin A, Tergitol NP-9®, and Citronella oil.

Another embodiment comprises cyclosporin A, Tergitol NP-9.5®, and Citronella oil.

Another embodiment comprises cyclosporin A, Tergitol TMN-10®, and Citronella oil.

Another embodiment comprises cyclosporin A, Tergitol TMN-3®, and Citronella oil.

Another embodiment comprises cyclosporin A, Tergitol TMN-6®, and Citronella oil.

Another embodiment comprises cyclosporin A, Tergitol XD®, and Citronella oil.

Another embodiment comprises cyclosporin A, Tergitol XH®, and Citronella oil.

Another embodiment comprises cyclosporin A, Tergitol XJ®, and Citronella oil.

Another embodiment comprises cyclosporin A, Tetronic 1107®, and Citronella oil.

Another embodiment comprises cyclosporin A, Tetronic 1307®, and Citronella oil.

Another embodiment comprises cyclosporin A, Tetronic 150R1®, and Citronella oil.

Another embodiment comprises cyclosporin A, Tetronic 304®, and Citronella oil.

Another embodiment comprises cyclosporin A, Tetronic 701®, and Citronella oil.

Another embodiment comprises cyclosporin A, Tetronic 901®, and Citronella oil.

Another embodiment comprises cyclosporin A, Tetronic 904®, and Citronella oil.

Another embodiment comprises cyclosporin A, Tetronic 908®, and Citronella oil.

Another embodiment comprises cyclosporin A, Tetronic 90R4®, and Citronella oil.

Another embodiment comprises cyclosporin A, Tomadol 1-3®, and Citronella oil.

Another embodiment comprises cyclosporin A, Tomadol 1-5®, and Citronella oil.

Another embodiment comprises cyclosporin A, Tomadol 1-7®, and Citronella oil.

Another embodiment comprises cyclosporin A, Tomadol 23-1®, and Citronella oil.

Another embodiment comprises cyclosporin A, Tomadol 23-3®, and Citronella oil.

Another embodiment comprises cyclosporin A, Tomadol 23-5®, and Citronella oil.

Another embodiment comprises cyclosporin A, Tomadol 23-6.5®, and Citronella oil.

Another embodiment comprises cyclosporin A, Tomadol 25-12®, and Citronella oil.

Another embodiment comprises cyclosporin A, Tomadol 25-3®, and Citronella oil.

Another embodiment comprises cyclosporin A, Tomadol 25-7®, and Citronella oil.

Another embodiment comprises cyclosporin A, Tomadol 25-9®, and Citronella oil.

Another embodiment comprises cyclosporin A, Tomadol 45-13®, and Citronella oil.

Another embodiment comprises cyclosporin A, Tomadol 45-2.5®, and Citronella oil.

Another embodiment comprises cyclosporin A, Tomadol 45-7®, and Citronella oil.

Another embodiment comprises cyclosporin A, Tomadol 91-2.5®, and Citronella oil.

Another embodiment comprises cyclosporin A, Tomadol 91-6®, and Citronella oil.

Another embodiment comprises cyclosporin A, Tomadol 91-8®, and Citronella oil.

Another embodiment comprises cyclosporin A, Triton X-100®, and Citronella oil.

Another embodiment comprises cyclosporin A, Triton X-102®, and Citronella oil.

Another embodiment comprises cyclosporin A, Triton X-114®, and Citronella oil.

Another embodiment comprises cyclosporin A, Triton X-15®, and Citronella oil.

Another embodiment comprises cyclosporin A, Triton X-165 (70%)®, and Citronella oil.

Another embodiment comprises cyclosporin A, Triton X-207®, and Citronella oil.

Another embodiment comprises cyclosporin A, Triton X-305 (70%)®, and Citronella oil.

Another embodiment comprises cyclosporin A, Triton X-35®, and Citronella oil.

Another embodiment comprises cyclosporin A, Triton X-405 (70%)®, and Citronella oil.

Another embodiment comprises cyclosporin A, Triton X-45®, and Citronella oil.

Another embodiment comprises cyclosporin A, Triton X-705 (70%)®, and Citronella oil.

Another embodiment comprises cyclosporin A, Volpo S-10®, and Citronella oil.

Another embodiment comprises cyclosporin A, Volpo S-2®, and Citronella oil.

Another embodiment comprises cyclosporin A, Volpo S-20®, and Citronella oil.

Another embodiment comprises cyclosporin A, Volpo-10®, and Citronella oil.

Another embodiment comprises cyclosporin A, Volpo-20®, and Citronella oil.

Another embodiment comprises cyclosporin A, Volpo-3®, and Citronella oil.

Another embodiment comprises cyclosporin A, Volpo-5®, and Citronella oil.

Another embodiment comprises cyclosporin A, Abex 2515®, and Juniper oil.

Another embodiment comprises cyclosporin A, Abex 2525/40®, and Juniper oil.

Another embodiment comprises cyclosporin A, Abex 2535®, and Juniper oil.

Another embodiment comprises cyclosporin A, Abex 2545®, and Juniper oil.

Another embodiment comprises cyclosporin A, Acconon E®, and Juniper oil.

Another embodiment comprises cyclosporin A, Alfonic 1216CO-9®, and Juniper oil.

Another embodiment comprises cyclosporin A, Alfonic 1412-3®, and Juniper oil.

Another embodiment comprises cyclosporin A, Alfonic TDA-12®, and Juniper oil.

Another embodiment comprises cyclosporin A, Alfonic TDA-3®, and Juniper oil.

Another embodiment comprises cyclosporin A, Alfonic TDA-4®, and Juniper oil.

Another embodiment comprises cyclosporin A, Alfonic TDA-6®, and Juniper oil.

Another embodiment comprises cyclosporin A, Alfonic TDA-7®, and Juniper oil.

Another embodiment comprises cyclosporin A, Alfonic TDA-8®, and Juniper oil.

Another embodiment comprises cyclosporin A, Alfonic TDA-9®, and Juniper oil.

Another embodiment comprises cyclosporin A, Alkamuls 400-DO®, and Juniper oil.

Another embodiment comprises cyclosporin A, Alkamuls 400-MO/E®, and Juniper oil.

Another embodiment comprises cyclosporin A, Ameroxol OE-10®, and Juniper oil.

Another embodiment comprises cyclosporin A, Ameroxol OE-2®, and Juniper oil.

Another embodiment comprises cyclosporin A, Ameroxol OE-20®, and Juniper oil.

Another embodiment comprises cyclosporin A, Armix 176®, and Juniper oil.

Another embodiment comprises cyclosporin A, Armix 180-C®, and Juniper oil.

Another embodiment comprises cyclosporin A, Armix 183®, and Juniper oil.

Another embodiment comprises cyclosporin A, Brij 35®, and Juniper oil.

Another embodiment comprises cyclosporin A, Brij 35 Liquid®, and Juniper oil.

Another embodiment comprises cyclosporin A, Brij 35 SP®, and Juniper oil.

Another embodiment comprises cyclosporin A, Canasol MJ 2109®, and Juniper oil.

Another embodiment comprises cyclosporin A, Canasol MJ 52®, and Juniper oil.

Another embodiment comprises cyclosporin A, Canasol R 3600®, and Juniper oil.

Another embodiment comprises cyclosporin A, Canasol R 3603®, and Juniper oil.

Another embodiment comprises cyclosporin A, Canasol R 4000 H®, and Juniper oil.

Another embodiment comprises cyclosporin A, Cedepal CA-210®, and Juniper oil.

Another embodiment comprises cyclosporin A, Cedepal CA-520®, and Juniper oil.

Another embodiment comprises cyclosporin A, Cedepal CA-630®, and Juniper oil.

Another embodiment comprises cyclosporin A, Cedepal CA-720®, and Juniper oil.

Another embodiment comprises cyclosporin A, Cedepal CA-890®, and Juniper oil.

Another embodiment comprises cyclosporin A, Cedepal CA-897®, and Juniper oil.

Another embodiment comprises cyclosporin A, Cedepal CO-210®, and Juniper oil.

Another embodiment comprises cyclosporin A, Cedepal CO-430®, and Juniper oil.

Another embodiment comprises cyclosporin A, Cedepal CO-530®, and Juniper oil.

Another embodiment comprises cyclosporin A, Cedepal CO-630®, and Juniper oil.

Another embodiment comprises cyclosporin A, Cedepal CO-710®, and Juniper oil.

Another embodiment comprises cyclosporin A, Cedepal CO-730®, and Juniper oil.

Another embodiment comprises cyclosporin A, Cedepal CO-880®, and Juniper oil.

Another embodiment comprises cyclosporin A, Cedepal CO-887®, and Juniper oil.

Another embodiment comprises cyclosporin A, Cedepal CO-890®, and Juniper oil.

Another embodiment comprises cyclosporin A, Cedepal CO-897®, and Juniper oil.

Another embodiment comprises cyclosporin A, Cedepal CO-970®, and Juniper oil.

Another embodiment comprises cyclosporin A, Cedepal CO-977®, and Juniper oil.

Another embodiment comprises cyclosporin A, Cedepal CO-990®, and Juniper oil.

Another embodiment comprises cyclosporin A, Cedepal CO-997®, and Juniper oil.

Another embodiment comprises cyclosporin A, Chemal BP 261®, and Juniper oil.

Another embodiment comprises cyclosporin A, Chemal BP 262®, and Juniper oil.

Another embodiment comprises cyclosporin A, Chemal BP 3172®, and Juniper oil.

Another embodiment comprises cyclosporin A, Chemal BP 3174®, and Juniper oil.

Another embodiment comprises cyclosporin A, Chemal DA-4®, and Juniper oil.

Another embodiment comprises cyclosporin A, Chemal DA-6®, and Juniper oil.

Another embodiment comprises cyclosporin A, Chemal DA-9®, and Juniper oil.

Another embodiment comprises cyclosporin A, Chemal LA-23®, and Juniper oil.

Another embodiment comprises cyclosporin A, Chemal LA-4®, and Juniper oil.

Another embodiment comprises cyclosporin A, Chemal LA-9®, and Juniper oil.

Another embodiment comprises cyclosporin A, Chemal OA-20G®, and Juniper oil.

Another embodiment comprises cyclosporin A, Chemal OA-5®, and Juniper oil.

Another embodiment comprises cyclosporin A, Chemal OA-9®, and Juniper oil.

Another embodiment comprises cyclosporin A, Chemal TDA-12®, and Juniper oil.

Another embodiment comprises cyclosporin A, Chemal TDA-15®, and Juniper oil.

Another embodiment comprises cyclosporin A, Chemal TDA-3®, and Juniper oil.

Another embodiment comprises cyclosporin A, Chemal TDA-6®, and Juniper oil.

Another embodiment comprises cyclosporin A, Chemal TDA-9®, and Juniper oil.

Another embodiment comprises cyclosporin A, Chemax CO-16®, and Juniper oil.

Another embodiment comprises cyclosporin A, Chemax CO-200/50®, and Juniper oil.

Another embodiment comprises cyclosporin A, Chemax CO-25®, and Juniper oil.

Another embodiment comprises cyclosporin A, Chemax CO-30®, and Juniper oil.

Another embodiment comprises cyclosporin A, Chemax CO-36®, and Juniper oil.

Another embodiment comprises cyclosporin A, Chemax CO-40®, and Juniper oil.

Another embodiment comprises cyclosporin A, Chemax CO-5®, and Juniper oil.

Another embodiment comprises cyclosporin A, Chemax CO-80®, and Juniper oil.

Another embodiment comprises cyclosporin A, Chemax DNP-150/50®, and Juniper oil.

Another embodiment comprises cyclosporin A, Chemax DNP-18®, and Juniper oil.

Another embodiment comprises cyclosporin A, Chemax DNP-8®, and Juniper oil.

Another embodiment comprises cyclosporin A, Chemax E 200 ML®, and Juniper oil.

Another embodiment comprises cyclosporin A, Chemax E 200 MO®, and Juniper oil.

Another embodiment comprises cyclosporin A, Chemax E 400 ML®, and Juniper oil.

Another embodiment comprises cyclosporin A, Chemax E 400 MO®, and Juniper oil.

Another embodiment comprises cyclosporin A, Chemax E 600 ML®, and Juniper oil.

Another embodiment comprises cyclosporin A, Chemax E 600 MO®, and Juniper oil.

Another embodiment comprises cyclosporin A, Chemax E 1000 MO®, and Juniper oil.

Another embodiment comprises cyclosporin A, Chemax E 1000 MS®, and Juniper oil.

Another embodiment comprises cyclosporin A, Chemax E200 MS®, and Juniper oil.

Another embodiment comprises cyclosporin A, Chemax E400 MS®, and Juniper oil.

Another embodiment comprises cyclosporin A, Chemax E600 MS®, and Juniper oil.

Another embodiment comprises cyclosporin A, Chemax HCO-16®, and Juniper oil.

Another embodiment comprises cyclosporin A, Chemax HCO-200/50®, and Juniper oil.

Another embodiment comprises cyclosporin A, Chemax HCO-25®, and Juniper oil.

Another embodiment comprises cyclosporin A, Chemax HCO-5®, and Juniper oil.

Another embodiment comprises cyclosporin A, Chemax TO-16®, and Juniper oil.

Another embodiment comprises cyclosporin A, Chemax TO-8®, and Juniper oil.

Another embodiment comprises cyclosporin A, Colalube 3409®, and Juniper oil.

Another embodiment comprises cyclosporin A, Colalube 3411®, and Juniper oil.

Another embodiment comprises cyclosporin A, Colalube 3413®, and Juniper oil.

Another embodiment comprises cyclosporin A, Colalube 3414®, and Juniper oil.

Another embodiment comprises cyclosporin A, Colalube 3415®, and Juniper oil.

Another embodiment comprises cyclosporin A, Colalube 3416®, and Juniper oil.

Another embodiment comprises cyclosporin A, Colalube 3417®, and Juniper oil.

Another embodiment comprises cyclosporin A, Delonic NPE-100®, and Juniper oil.

Another embodiment comprises cyclosporin A, Delonic NPE-30®, and Juniper oil.

Another embodiment comprises cyclosporin A, Delonic NPE-40®, and Juniper oil.

Another embodiment comprises cyclosporin A, Delonic OPE-10®, and Juniper oil.

Another embodiment comprises cyclosporin A, Delonic OPE-12®, and Juniper oil.

Another embodiment comprises cyclosporin A, Delonic OPE-30®, and Juniper oil.

Another embodiment comprises cyclosporin A, Delonic OPE-40®, and Juniper oil.

Another embodiment comprises cyclosporin A, Delonic OPE-5®, and Juniper oil.

Another embodiment comprises cyclosporin A, Delonic OPE-7.5®, and Juniper oil.

Another embodiment comprises cyclosporin A, DePeg 16-CO®, and Juniper oil.

Another embodiment comprises cyclosporin A, DePeg 25-CO®, and Juniper oil.

Another embodiment comprises cyclosporin A, DePeg 30-CO®, and Juniper oil.

Another embodiment comprises cyclosporin A, DePeg 40-CO®, and Juniper oil.

Another embodiment comprises cyclosporin A, DePeg 5-CO®, and Juniper oil.

Another embodiment comprises cyclosporin A, DePeg 80-CO®, and Juniper oil.

Another embodiment comprises cyclosporin A, DeSonic S-100®, and Juniper oil.

Another embodiment comprises cyclosporin A, DeSonic S-114®, and Juniper oil.

Another embodiment comprises cyclosporin A, DeSonic S-405®, and Juniper oil.

Another embodiment comprises cyclosporin A, DeSonic S-45®, and Juniper oil.

Another embodiment comprises cyclosporin A, DeSonic TDA-9®, and Juniper oil.

Another embodiment comprises cyclosporin A, DeThox Acid O-14®, and Juniper oil.

Another embodiment comprises cyclosporin A, DeThox Acid O-16®, and Juniper oil.

Another embodiment comprises cyclosporin A, DeThox Acid O-9®, and Juniper oil.

Another embodiment comprises cyclosporin A, DeThox Acid TO-14®, and Juniper oil.

Another embodiment comprises cyclosporin A, DeThox Acid TO-16.5®, and Juniper oil.

Another embodiment comprises cyclosporin A, DeThox Acid TO-8.5®, and Juniper oil.

Another embodiment comprises cyclosporin A, Dur-Em 114®, and Juniper oil.

Another embodiment comprises cyclosporin A, Dur-Em 117®, and Juniper oil.

Another embodiment comprises cyclosporin A, Dur-Em 204®, and Juniper oil.

Another embodiment comprises cyclosporin A, Dur-Em 207®, and Juniper oil.

Another embodiment comprises cyclosporin A, Dur-Em 207E®, and Juniper oil.

Another embodiment comprises cyclosporin A, Emerest 2704®, and Juniper oil.

Another embodiment comprises cyclosporin A, Emerest 2712®, and Juniper oil.

Another embodiment comprises cyclosporin A, Emerest 2715®, and Juniper oil.

Another embodiment comprises cyclosporin A, Ethal EH-2®, and Juniper oil.

Another embodiment comprises cyclosporin A, Ethal EH-5®, and Juniper oil.

Another embodiment comprises cyclosporin A, Ethal OA-23®, and Juniper oil.

Another embodiment comprises cyclosporin A, Ethosperse CA-20®, and Juniper oil.

Another embodiment comprises cyclosporin A, Ethosperse LA-12®, and Juniper oil.

Another embodiment comprises cyclosporin A, Ethosperse LA-23®, and Juniper oil.

Another embodiment comprises cyclosporin A, Ethosperse LA-4®, and Juniper oil.

Another embodiment comprises cyclosporin A, Ethox 3095®, and Juniper oil.

Another embodiment comprises cyclosporin A, Ethox CO-16®, and Juniper oil.

Another embodiment comprises cyclosporin A, Ethox CO-200®, and Juniper oil.

Another embodiment comprises cyclosporin A, Ethox CO-25®, and Juniper oil.

Another embodiment comprises cyclosporin A, Ethox CO-30®, and Juniper oil.

Another embodiment comprises cyclosporin A, Ethox CO-36®, and Juniper oil.

Another embodiment comprises cyclosporin A, Ethox CO-40®, and Juniper oil.

Another embodiment comprises cyclosporin A, Ethox CO-5®, and Juniper oil.

Another embodiment comprises cyclosporin A, Ethox DO-14®, and Juniper oil.

Another embodiment comprises cyclosporin A, Ethox DO-9®, and Juniper oil.

Another embodiment comprises cyclosporin A, Ethox MA-8®, and Juniper oil.

Another embodiment comprises cyclosporin A, Ethox MI-14®, and Juniper oil.

Another embodiment comprises cyclosporin A, Ethox ML-14®, and Juniper oil.

Another embodiment comprises cyclosporin A, Ethox ML-5®, and Juniper oil.

Another embodiment comprises cyclosporin A, Ethox MO-14®, and Juniper oil.

Another embodiment comprises cyclosporin A, Ethox MO-9®, and Juniper oil.

Another embodiment comprises cyclosporin A, Ethox MS-14®, and Juniper oil.

Another embodiment comprises cyclosporin A, Ethox MS-23®, and Juniper oil.

Another embodiment comprises cyclosporin A, Ethox MS-40®, and Juniper oil.

Another embodiment comprises cyclosporin A, Ethox MS-8®, and Juniper oil.

Another embodiment comprises cyclosporin A, Ethox TO-16®, and Juniper oil.

Another embodiment comprises cyclosporin A, Ethox TO-8®, and Juniper oil.

Another embodiment comprises cyclosporin A, Ethylan SN-120®, and Juniper oil.

Another embodiment comprises cyclosporin A, Ethylan SN-70®, and Juniper oil.

Another embodiment comprises cyclosporin A, Ethylan SN-90®, and Juniper oil.

Another embodiment comprises cyclosporin A, Flexricin 13®, and Juniper oil.

Another embodiment comprises cyclosporin A, Flexricin 15®, and Juniper oil.

Another embodiment comprises cyclosporin A, Genapol 1261®, and Juniper oil.

Another embodiment comprises cyclosporin A, Generol 122 E25®, and Juniper oil.

Another embodiment comprises cyclosporin A, Glicepol 160®, and Juniper oil.

Another embodiment comprises cyclosporin A, Glicepol 560®, and Juniper oil.

Another embodiment comprises cyclosporin A, Glicepol GMS 20®, and Juniper oil.

Another embodiment comprises cyclosporin A, Hetoxide BN-13®, and Juniper oil.

Another embodiment comprises cyclosporin A, Hetoxide DNP-4®, and Juniper oil.

Another embodiment comprises cyclosporin A, Hetoxide DNP-9.6®, and Juniper oil.

Another embodiment comprises cyclosporin A, Hetoxide G-26®, and Juniper oil.

Another embodiment comprises cyclosporin A, Hetoxide G-7®, and Juniper oil.

Another embodiment comprises cyclosporin A, Hetoxide GT-20®, and Juniper oil.

Another embodiment comprises cyclosporin A, Hetoxide HC-40®, and Juniper oil.

Another embodiment comprises cyclosporin A, Hetoxol C-24®, and Juniper oil.

Another embodiment comprises cyclosporin A, Hetoxol CA-10®, and Juniper oil.

Another embodiment comprises cyclosporin A, Hetoxol CA-2®, and Juniper oil.

Another embodiment comprises cyclosporin A, Hetoxol CA-20®, and Juniper oil.

Another embodiment comprises cyclosporin A, Hetoxol CAWS®, and Juniper oil.

Another embodiment comprises cyclosporin A, Hetoxol CD-3®, and Juniper oil.

Another embodiment comprises cyclosporin A, Hetoxol CD-4®, and Juniper oil.

Another embodiment comprises cyclosporin A, Hetoxol CS-15®, and Juniper oil.

Another embodiment comprises cyclosporin A, Hetoxol CS-20®, and Juniper oil.

Another embodiment comprises cyclosporin A, Hetoxol CS-20D®, and Juniper oil.

Another embodiment comprises cyclosporin A, Hetoxol CS-25®, and Juniper oil.

Another embodiment comprises cyclosporin A, Hetoxol CS-50®., and Juniper oil.

Another embodiment comprises cyclosporin A, Hetoxol CS-9®, and Juniper oil.

Another embodiment comprises cyclosporin A, Hetoxol CSA-15®, and Juniper oil.

Another embodiment comprises cyclosporin A, Hetoxol D®, and Juniper oil.

Another embodiment comprises cyclosporin A, Hetoxol G®, and Juniper oil.

Another embodiment comprises cyclosporin A, Hetoxol J®, and Juniper oil.

Another embodiment comprises cyclosporin A, Hetoxol L®, and Juniper oil.

Another embodiment comprises cyclosporin A, Hetoxol L-23®, and Juniper oil.

Another embodiment comprises cyclosporin A, Hetoxol L-4®, and Juniper oil.

Another embodiment comprises cyclosporin A, Hetoxol L-9®, and Juniper oil.

Another embodiment comprises cyclosporin A, Hetoxol LS-9®, and Juniper oil.

Another embodiment comprises cyclosporin A, Hetoxol M-3®, and Juniper oil.

Another embodiment comprises cyclosporin A, Hetoxol OA-10 Special®, and Juniper oil.

Another embodiment comprises cyclosporin A, Hetoxol OA-20 Specialp, and Juniper oil.

Another embodiment comprises cyclosporin A, Hetoxol OA-3 Special®, and Juniper oil.

Another embodiment comprises cyclosporin A, Hetoxol OA-5 Special®, and Juniper oil.

Another embodiment comprises cyclosporin A, Hetoxol OL-10®, and Juniper oil.

Another embodiment comprises cyclosporin A, Hetoxol OL-2®, and Juniper oil.

Another embodiment comprises cyclosporin A, Hetoxol OL-23®, and Juniper oil.

Another embodiment comprises cyclosporin A, Hetoxol OL-4®, and Juniper oil.

Another embodiment comprises cyclosporin A, Hetoxol OL-40®, and Juniper oil.

Another embodiment comprises cyclosporin A, Hetoxol OL-5®, and Juniper oil.

Another embodiment comprises cyclosporin A, Hetoxol PLA®, and Juniper oil.

Another embodiment comprises cyclosporin A, Hetoxol SP-15®, and Juniper oil.

Another embodiment comprises cyclosporin A, Hetoxol STA-10®, and Juniper oil.

Another embodiment comprises cyclosporin A, Hetoxol STA-2®, and Juniper oil.

Another embodiment comprises cyclosporin A, Hetoxol STA-30®, and Juniper oil.

Another embodiment comprises cyclosporin A, Hetoxol TD-12®, and Juniper oil.

Another embodiment comprises cyclosporin A, Hetoxol TD-18®, and Juniper oil.

Another embodiment comprises cyclosporin A, Hetoxol TD-3®, and Juniper oil.

Another embodiment comprises cyclosporin A, Hetoxol TD-6®, and Juniper oil.

Another embodiment comprises cyclosporin A, Hetoxol TD-9®, and Juniper oil.

Another embodiment comprises cyclosporin A, Hetoxol TDEP-15®, and Juniper oil.

Another embodiment comprises cyclosporin A, Hetoxol TDEP-63®, and Juniper oil.

Another embodiment comprises cyclosporin A, Iconol 24-12®, and Juniper oil.

Another embodiment comprises cyclosporin A, Iconol 24-23®, and Juniper oil.

Another embodiment comprises cyclosporin A, Iconol 24-4®, and Juniper oil.

Another embodiment comprises cyclosporin A, Iconol DA-4®, and Juniper oil.

Another embodiment comprises cyclosporin A, Iconol DA-6®, and Juniper oil.

Another embodiment comprises cyclosporin A, Iconol TDA-10®, and Juniper oil.

Another embodiment comprises cyclosporin A, Iconol TDA-3®, and Juniper oil.

Another embodiment comprises cyclosporin A, Iconol TDA-6®, and Juniper oil.

Another embodiment comprises cyclosporin A, Iconol TDA-8-90%®, and Juniper oil.

Another embodiment comprises cyclosporin A, Iconol TDA-9®, and Juniper oil.

Another embodiment comprises cyclosporin A, Igepal CA-210®, and Juniper oil.

Another embodiment comprises cyclosporin A, Igepal CA-407®, and Juniper oil.

Another embodiment comprises cyclosporin A, Igepal CA-420®, and Juniper oil.

Another embodiment comprises cyclosporin A, Igepal CA-520®, and Juniper oil.

Another embodiment comprises cyclosporin A, Igepal CA-620®, and Juniper oil.

Another embodiment comprises cyclosporin A, Igepal CA-630®, and Juniper oil.

Another embodiment comprises cyclosporin A, Igepal CA-720®, and Juniper oil.

Another embodiment comprises cyclosporin A, Igepal CA-877®, and Juniper oil.

Another embodiment comprises cyclosporin A, Igepal CA-887®, and Juniper oil.

Another embodiment comprises cyclosporin A, Igepal CA-897®, and Juniper oil.

Another embodiment comprises cyclosporin A, Igepal CO-210®, and Juniper oil.

Another embodiment comprises cyclosporin A, Igepal CO-430®, and Juniper oil.

Another embodiment comprises cyclosporin A, Igepal CO-520®, and Juniper oil.

Another embodiment comprises cyclosporin A, Igepal CO-530®, and Juniper oil.

Another embodiment comprises cyclosporin A, Igepal CO-610®, and Juniper oil.

Another embodiment comprises cyclosporin A, Igepal CO-630®, and Juniper oil.

Another embodiment comprises cyclosporin A, Igepal CO-630 Special®, and Juniper oil.

Another embodiment comprises cyclosporin A, Igepal CO-660®, and Juniper oil.

Another embodiment comprises cyclosporin A, Igepal CO-710®, and Juniper oil.

Another embodiment comprises cyclosporin A, Igepal CO-720®, and Juniper oil.

Another embodiment comprises cyclosporin A, Igepal CO-730®, and Juniper oil.

Another embodiment comprises cyclosporin A, Igepal CO-738®, and Juniper oil.

Another embodiment comprises cyclosporin A, Igepal CO-850®, and Juniper oil.

Another embodiment comprises cyclosporin A, Igepal CO-880®, and Juniper oil.

Another embodiment comprises cyclosporin A, Igepal CO-887®, and Juniper oil.

Another embodiment comprises cyclosporin A, Igepal CO-890®, and Juniper oil.

Another embodiment comprises cyclosporin A, Igepal CO-897®, and Juniper oil.

Another embodiment comprises cyclosporin A, Igepal CO-970®, and Juniper oil.

Another embodiment comprises cyclosporin A, Igepal CO-977®, and Juniper oil.

Another embodiment comprises cyclosporin A, Igepal CO-987®, and Juniper oil.

Another embodiment comprises cyclosporin A, Igepal CO-990 FLK®, and Juniper oil.

Another embodiment comprises cyclosporin A, Igepal CO-997®, and Juniper oil.

Another embodiment comprises cyclosporin A, Igepal DM-430®, and Juniper oil.

Another embodiment comprises cyclosporin A, Igepal DM-530®, and Juniper oil.

Another embodiment comprises cyclosporin A, Igepal DM-710®, and Juniper oil.

Another embodiment comprises cyclosporin A, Igepal DM-730®, and Juniper oil.

Another embodiment comprises cyclosporin A, Igepal DM-880®, and Juniper oil.

Another embodiment comprises cyclosporin A, Igepal DM-970 FLK®, and Juniper oil.

Another embodiment comprises cyclosporin A, Igepal OD-410®, and Juniper oil.

Another embodiment comprises cyclosporin A, Igepal RC-520®, and Juniper oil.

Another embodiment comprises cyclosporin A, Igepal RC-620®, and Juniper oil.

Another embodiment comprises cyclosporin A, Igepal RC-630®, and Juniper oil.

Another embodiment comprises cyclosporin A, Lexemul CS-20®, and Juniper oil.

Another embodiment comprises cyclosporin A, Lipo GMS 450®, and Juniper oil.

Another embodiment comprises cyclosporin A, Lipo Polyglycol 1000®, and Juniper oil.

Another embodiment comprises cyclosporin A, Lipo Polyglycol 200®, and Juniper oil.

Another embodiment comprises cyclosporin A, Lipo Polyglycol 300®, and Juniper oil.

Another embodiment comprises cyclosporin A, Lipo Polyglycol 3350®, and Juniper oil.

Another embodiment comprises cyclosporin A, Lipo Polyglycol 400®, and Juniper oil.

Another embodiment comprises cyclosporin A, Lipo Polyglycol 600®, and Juniper oil.

Another embodiment comprises cyclosporin A, Lipocol C-10®, and Juniper oil.

Another embodiment comprises cyclosporin A, Lipocol C-2®, and Juniper oil.

Another embodiment comprises cyclosporin A, Lipocol C-20®, and Juniper oil.

Another embodiment comprises cyclosporin A, Lipocol L-12®, and Juniper oil.

Another embodiment comprises cyclosporin A, Lipocol L-23®, and Juniper oil.

Another embodiment comprises cyclosporin A, Lipocol L-4®, and Juniper oil.

Another embodiment comprises cyclosporin A, Lipocol O-10®, and Juniper oil.

Another embodiment comprises cyclosporin A, Lipocol O-2®, and Juniper oil.

Another embodiment comprises cyclosporin A, Lipocol O-20®, and Juniper oil.

Another embodiment comprises cyclosporin A, Lipocol O-5®, and Juniper oil.

Another embodiment comprises cyclosporin A, Lipocol S-10®, and Juniper oil.

Another embodiment comprises cyclosporin A, Lipocol S-2®, and Juniper oil.

Another embodiment comprises cyclosporin A, Lipocol S-20®, and Juniper oil.

Another embodiment comprises cyclosporin A, Lipocol SC-15®, and Juniper oil.

Another embodiment comprises cyclosporin A, Lipocol SC-20®, and Juniper oil.

Another embodiment comprises cyclosporin A, Lipocol SC-4®, and Juniper oil.

Another embodiment comprises cyclosporin A, Lipocol TD-12®, and Juniper oil.

Another embodiment comprises cyclosporin A, Lumulse 40-O K®, and Juniper oil.

Another embodiment comprises cyclosporin A, Lumulse 42-O K®, and Juniper oil.

Another embodiment comprises cyclosporin A, Lumulse GMT-40®, and Juniper oil.

Another embodiment comprises cyclosporin A, Lumulse PEG 1450®, and Juniper oil.

Another embodiment comprises cyclosporin A, Lumulse PEG 1450 NF®, and Juniper oil.

Another embodiment comprises cyclosporin A, Lumulse PEG 8000®, and Juniper oil.

Another embodiment comprises cyclosporin A, Lumulse PGO®, and Juniper oil.

Another embodiment comprises cyclosporin A, Lumulse POE (2) Oleyl Amine®, and Juniper oil.

Another embodiment comprises cyclosporin A, Magrabar PGO-104®, and Juniper oil.

Another embodiment comprises cyclosporin A, Mapeg 200 ML®, and Juniper oil.

Another embodiment comprises cyclosporin A, Mapeg 400 MO®, and Juniper oil.

Another embodiment comprises cyclosporin A, Mapeg 400 MOT®, and Juniper oil.

Another embodiment comprises cyclosporin A, Mapeg 400DO®, and Juniper oil.

Another embodiment comprises cyclosporin A, Mapeg 400DOT®, and Juniper oil.

Another embodiment comprises cyclosporin A, Mapeg 600DOT®, and Juniper oil.

Another embodiment comprises cyclosporin A, Marlox B 24/80®, and Juniper oil.

Another embodiment comprises cyclosporin A, Marlox FK 86®, and Juniper oil.

Another embodiment comprises cyclosporin A, Marlox MO 154®, and Juniper oil.

Another embodiment comprises cyclosporin A, Mazox CDA CG®, and Juniper oil.

Another embodiment comprises cyclosporin A, Monoglycerides Glyceryl Monestearate®, and Juniper oil.

Another embodiment comprises cyclosporin A, Myrj 45®, and Juniper oil.

Another embodiment comprises cyclosporin A, Myrj 52®, and Juniper oil.

Another embodiment comprises cyclosporin A, Myrj 52S®, and Juniper oil.

Another embodiment comprises cyclosporin A, Myrj 53®, and Juniper oil.

Another embodiment comprises cyclosporin A, Myrj 59®, and Juniper oil.

Another embodiment comprises cyclosporin A, Norfox NP-12®, and Juniper oil.

Another embodiment comprises cyclosporin A, Norfox NP-4®, and Juniper oil.

Another embodiment comprises cyclosporin A, Norfox NP-6®, and Juniper oil.

Another embodiment comprises cyclosporin A, Norfox NP-887®, and Juniper oil.

Another embodiment comprises cyclosporin A, Norfox NP-9®, and Juniper oil.

Another embodiment comprises cyclosporin A, Norfox NP-977®, and Juniper oil.

Another embodiment comprises cyclosporin A, Norfox OP-100®, and Juniper oil.

Another embodiment comprises cyclosporin A, Norfox OP-102®, and Juniper oil.

Another embodiment comprises cyclosporin A, Norfox OP-114®, and Juniper oil.

Another embodiment comprises cyclosporin A, Norfox OP-45®, and Juniper oil.

Another embodiment comprises cyclosporin A, Pegosperse 100-L®, and Juniper oil.

Another embodiment comprises cyclosporin A, Pegosperse 100-O®, and Juniper oil.

Another embodiment comprises cyclosporin A, Pegosperse 100-S®, and Juniper oil.

Another embodiment comprises cyclosporin A, Pegosperse 1500-MS®, and Juniper oil.

Another embodiment comprises cyclosporin A, Pegosperse 1750-MS®, and Juniper oil.

Another embodiment comprises cyclosporin A, Pegosperse 200 DL®, and Juniper oil.

Another embodiment comprises cyclosporin A, Pegosperse 200-ML®, and Juniper oil.

Another embodiment comprises cyclosporin A, Pegosperse 400-DL®, and Juniper oil.

Another embodiment comprises cyclosporin A, Pegosperse 400-DO®, and Juniper oil.

Another embodiment comprises cyclosporin A, Pegosperse 400-DS®, and Juniper oil.

Another embodiment comprises cyclosporin A, Pegosperse 400-ML®, and Juniper oil.

Another embodiment comprises cyclosporin A, Pegosperse 400-MO®, and Juniper oil.

Another embodiment comprises cyclosporin A, Pegosperse 400-MOT®, and Juniper oil.

Another embodiment comprises cyclosporin A, Pegosperse 400-MS®, and Juniper oil.

Another embodiment comprises cyclosporin A, Pegosperse 50-DS®, and Juniper oil.

Another embodiment comprises cyclosporin A, Pegosperse 50-MS®, and Juniper oil.

Another embodiment comprises cyclosporin A, Pegosperse 600-ML®, and Juniper oil.

Another embodiment comprises cyclosporin A, Pegosperse 600-MS®, and Juniper oil.

Another embodiment comprises cyclosporin A, Plurafac A-38®, and Juniper oil.

Another embodiment comprises cyclosporin A, Plurafac B-25-5®, and Juniper oil.

Another embodiment comprises cyclosporin A, Plurafac B-26®, and Juniper oil.

Another embodiment comprises cyclosporin A, Plurafac D-25®, and Juniper oil.

Another embodiment comprises cyclosporin A, Plurafac LF 131®, and Juniper oil.

Another embodiment comprises cyclosporin A, Plurafac LF 4030®, and Juniper oil.

Another embodiment comprises cyclosporin A, Plurafac LF 7000®, and Juniper oil.

Another embodiment comprises cyclosporin A, Plurafac RA-20®, and Juniper oil.

Another embodiment comprises cyclosporin A, Plurafac RA-30®, and Juniper oil.

Another embodiment comprises cyclosporin A, Plurafac RA-40®, and Juniper oil.

Another embodiment comprises cyclosporin A, Pluronic 10R5®, and Juniper oil.

Another embodiment comprises cyclosporin A, Pluronic 17R2®, and Juniper oil.

Another embodiment comprises cyclosporin A, Pluronic 17R4®, and Juniper oil.

Another embodiment comprises cyclosporin A, Pluronic 25R2®, and Juniper oil.

Another embodiment comprises cyclosporin A, Pluronic 25R4®, and Juniper oil.

Another embodiment comprises cyclosporin A, Pluronic 31R1®, and Juniper oil.

Another embodiment comprises cyclosporin A, Pluronic F108®, and Juniper oil.

Another embodiment comprises cyclosporin A, Pluronic F127®, and Juniper oil.

Another embodiment comprises cyclosporin A, Pluronic F38®, and Juniper oil.

Another embodiment comprises cyclosporin A, Pluronic F68®, and Juniper oil.

Another embodiment comprises cyclosporin A, Pluronic F68LF®, and Juniper oil.

Another embodiment comprises cyclosporin A, Pluronic F77®, and Juniper oil.

Another embodiment comprises cyclosporin A, Pluronic F87®, and Juniper oil.

Another embodiment comprises cyclosporin A, Pluronic F88®, and Juniper oil.

Another embodiment comprises cyclosporin A, Pluronic F98®, and Juniper oil.

Another embodiment comprises cyclosporin A, Pluronic L10®, and Juniper oil.

Another embodiment comprises cyclosporin A, Pluronic L101®, and Juniper oil.

Another embodiment comprises cyclosporin A, Pluronic L121®, and Juniper oil.

Another embodiment comprises cyclosporin A, Pluronic L31®, and Juniper oil.

Another embodiment comprises cyclosporin A, Pluronic L35®, and Juniper oil.

Another embodiment comprises cyclosporin A, Pluronic L43®, and Juniper oil.

Another embodiment comprises cyclosporin A, Pluronic L44®, and Juniper oil.

Another embodiment comprises cyclosporin A, Pluronic L61®, and Juniper oil.

Another embodiment comprises cyclosporin A, Pluronic L62®, and Juniper oil.

Another embodiment comprises cyclosporin A, Pluronic L62D®, and Juniper oil.

Another embodiment comprises cyclosporin A, Pluronic L62LF®, and Juniper oil.

Another embodiment comprises cyclosporin A, Pluronic L64®, and Juniper oil.

Another embodiment comprises cyclosporin A, Pluronic L81®, and Juniper oil.

Another embodiment comprises cyclosporin A, Pluronic L92®, and Juniper oil.

Another embodiment comprises cyclosporin A, Pluronic P103®, and Juniper oil.

Another embodiment comprises cyclosporin A, Pluronic P104®, and Juniper oil.

Another embodiment comprises cyclosporin A, Pluronic P105®, and Juniper oil.

Another embodiment comprises cyclosporin A, Pluronic P123®, and Juniper oil.

Another embodiment comprises cyclosporin A, Pluronic P65®, and Juniper oil.

Another embodiment comprises cyclosporin A, Pluronic P84®, and Juniper oil.

Another embodiment comprises cyclosporin A, Pluronic P85®, and Juniper oil.

Another embodiment comprises cyclosporin A, Polyaldo 10-2-P®, and Juniper oil.

Another embodiment comprises cyclosporin A, Polyaldo DGDO®, and Juniper oil.

Another embodiment comprises cyclosporin A, Polyaldo HGDS®, and Juniper oil.

Another embodiment comprises cyclosporin A, Polyaldo TGMS®, and Juniper oil.

Another embodiment comprises cyclosporin A, Protegin V®, and Juniper oil.

Another embodiment comprises cyclosporin A, Protegin W®, and Juniper oil.

Another embodiment comprises cyclosporin A, Protegin WX®, and Juniper oil.

Another embodiment comprises cyclosporin A, Protegin XV®, and Juniper oil.

Another embodiment comprises cyclosporin A, Rhodasurf BC-420®, and Juniper oil.

Another embodiment comprises cyclosporin A, Rhodasurf BC-610®, and Juniper oil.

Another embodiment comprises cyclosporin A, Rhodasurf BC-630®, and Juniper oil.

Another embodiment comprises cyclosporin A, Rhodasurf BC-720®, and Juniper oil.

Another embodiment comprises cyclosporin A, Rhodasurf BC-840®, and Juniper oil.

Another embodiment comprises cyclosporin A, Rhodasurf L 1®, and Juniper oil.

Another embodiment comprises cyclosporin A, Rhodasurf L 12®, and Juniper oil.

Another embodiment comprises cyclosporin A, Rhodasurf L 2®, and Juniper oil.

Another embodiment comprises cyclosporin A, Rhodasurf L 20®, and Juniper oil.

Another embodiment comprises cyclosporin A, Rhodasurf L 3®, and Juniper oil.

Another embodiment comprises cyclosporin A, Rhodasurf L 30®, and Juniper oil.

Another embodiment comprises cyclosporin A, Rhodasurf L 4®, and Juniper oil.

Another embodiment comprises cyclosporin A, Rhodasurf L 50®, and Juniper oil.

Another embodiment comprises cyclosporin A, Rhodasurf L 7/90®, and Juniper oil.

Another embodiment comprises cyclosporin A, Rhodasurf LA-3®, and Juniper oil.

Another embodiment comprises cyclosporin A, Rhodasurf LA-7®, and Juniper oil.

Another embodiment comprises cyclosporin A, Rhodasurf LAN-23®, and Juniper oil.

Another embodiment comprises cyclosporin A, Rhodasurf ON-870®, and Juniper oil.

Another embodiment comprises cyclosporin A, Rhodasurf ON-877®, and Juniper oil.

Another embodiment comprises cyclosporin A, Rhodasurf TDA-8/5®, and Juniper oil.

Another embodiment comprises cyclosporin A, Ritapro 100®, and Juniper oil.

Another embodiment comprises cyclosporin A, Ritapro 165®, and Juniper oil.

Another embodiment comprises cyclosporin A, Ritapro 200®, and Juniper oil.

Another embodiment comprises cyclosporin A, Ritapro 300®, and Juniper oil.

Another embodiment comprises cyclosporin A, Ritoleth 10®, and Juniper oil.

Another embodiment comprises cyclosporin A, Ritoleth 2®, and Juniper oil.

Another embodiment comprises cyclosporin A, Ritoleth 20®, and Juniper oil.

Another embodiment comprises cyclosporin A, Ritoleth 5®, and Juniper oil.

Another embodiment comprises cyclosporin A, Santone 10-10-0®, and Juniper oil.

Another embodiment comprises cyclosporin A, Santone 3-1-S XTR®, and Juniper oil.

Another embodiment comprises cyclosporin A, Santone 3-1-SH®, and Juniper oil.

Another embodiment comprises cyclosporin A, Santone 8-1-O®, and Juniper oil.

Another embodiment comprises cyclosporin A, Solulan 16®, and Juniper oil.

Another embodiment comprises cyclosporin A, Solulan 75®, and Juniper oil.

Another embodiment comprises cyclosporin A, Solulan C-24®, and Juniper oil.

Another embodiment comprises cyclosporin A, Solulan L-575®, and Juniper oil.

Another embodiment comprises cyclosporin A, Standamox CAW®, and Juniper oil.

Another embodiment comprises cyclosporin A, Surfine AZI-A®, and Juniper oil.

Another embodiment comprises cyclosporin A, Surfonic CO-15®, and Juniper oil.

Another embodiment comprises cyclosporin A, Surfonic CO-25®, and Juniper oil.

Another embodiment comprises cyclosporin A, Surfonic CO-30®, and Juniper oil.

Another embodiment comprises cyclosporin A, Surfonic CO-36®, and Juniper oil.

Another embodiment comprises cyclosporin A, Surfonic CO-42®, and Juniper oil.

Another embodiment comprises cyclosporin A, Surfonic E400-MO®, and Juniper oil.

Another embodiment comprises cyclosporin A, Surfonic L12-3®, and Juniper oil.

Another embodiment comprises cyclosporin A, Surfonic L12-6®, and Juniper oil.

Another embodiment comprises cyclosporin A, Surfonic L12-8®, and Juniper oil.

Another embodiment comprises cyclosporin A, Surfonic L24-12®, and Juniper oil.

Another embodiment comprises cyclosporin A, Surfonic L24-2®, and Juniper oil.

Another embodiment comprises cyclosporin A, Surfonic L24-22®, and Juniper oil.

Another embodiment comprises cyclosporin A, Surfonic L24-3®, and Juniper oil.

Another embodiment comprises cyclosporin A, Surfonic L24-4®, and Juniper oil.

Another embodiment comprises cyclosporin A, Surfonic L24-5®, and Juniper oil.

Another embodiment comprises cyclosporin A, Surfonic L24-7®, and Juniper oil.

Another embodiment comprises cyclosporin A, Surfonic L24-7.1®, and Juniper oil.

Another embodiment comprises cyclosporin A, Surfonic L24-9®, and Juniper oil.

Another embodiment comprises cyclosporin A, Surfonic LF-17®, and Juniper oil.

Another embodiment comprises cyclosporin A, Surfonic LF-18®, and Juniper oil.

Another embodiment comprises cyclosporin A, Surfonic LF-37®, and Juniper oil.

Another embodiment comprises cyclosporin A, Surfonic LF-41®, and Juniper oil.

Another embodiment comprises cyclosporin A, Surfonic LF-42®, and Juniper oil.

Another embodiment comprises cyclosporin A, Surfonic LF-47®, and Juniper oil.

Another embodiment comprises cyclosporin A, Surfonic LF-68®, and Juniper oil.

Another embodiment comprises cyclosporin A, Surfonic N-10®, and Juniper oil.

Another embodiment comprises cyclosporin A, Surfonic N-100®, and Juniper oil.

Another embodiment comprises cyclosporin A, Surfonic N-102®, and Juniper oil.

Another embodiment comprises cyclosporin A, Surfonic N-120®, and Juniper oil.

Another embodiment comprises cyclosporin A, Surfonic N-150®, and Juniper oil.

Another embodiment comprises cyclosporin A, Surfonic N-200®, and Juniper oil.

Another embodiment comprises cyclosporin A, Surfonic N-300®, and Juniper oil.

Another embodiment comprises cyclosporin A, Surfonic N-31.5®, and Juniper oil.

Another embodiment comprises cyclosporin A, Surfonic N-40®, and Juniper oil.

Another embodiment comprises cyclosporin A, Surfonic N-400®, and Juniper oil.

Another embodiment comprises cyclosporin A, Surfonic N-60®, and Juniper oil.

Another embodiment comprises cyclosporin A, Surfonic N-85®, and Juniper oil.

Another embodiment comprises cyclosporin A, Surfonic N-95®, and Juniper oil.

Another embodiment comprises cyclosporin A, Surfonic NB-18®, and Juniper oil.

Another embodiment comprises cyclosporin A, Surfonic NB-23®, and Juniper oil.

Another embodiment comprises cyclosporin A, Surfonic NB-25®, and Juniper oil.

Another embodiment comprises cyclosporin A, Surfonic NB-307®, and Juniper oil.

Another embodiment comprises cyclosporin A, Surfonic NB-31®, and Juniper oil.

Another embodiment comprises cyclosporin A, Surfonic NB-41®, and Juniper oil.

Another embodiment comprises cyclosporin A, Surfonic NB-43®, and Juniper oil.

Another embodiment comprises cyclosporin A, Surfonic OP-100®, and Juniper oil.

Another embodiment comprises cyclosporin A, Surlonic OP-120®, and Juniper oil.

Another embodiment comprises cyclosporin A, Surlonic OP-15®, and Juniper oil.

Another embodiment comprises cyclosporin A, Surfonic OP-35®, and Juniper oil.

Another embodiment comprises cyclosporin A, Surlonic OP-70®, and Juniper oil.

Another embodiment comprises cyclosporin A, Surfonic P1®, and Juniper oil.

Another embodiment comprises cyclosporin A, Surlonic P3®, and Juniper oil.

Another embodiment comprises cyclosporin A, Surfonic P5®, and Juniper oil.

Another embodiment comprises cyclosporin A, Surlonic P6®, and Juniper oil.

Another embodiment comprises cyclosporin A, Surfonic POA-17R2®, and Juniper oil.

Another embodiment comprises cyclosporin A, Surfonic POA-25R2®, and Juniper oil.

Another embodiment comprises cyclosporin A, Surfonic TDA-11®, and Juniper oil.

Another embodiment comprises cyclosporin A, Surlonic TDA-3B®, and Juniper oil.

Another embodiment comprises cyclosporin A, Surfonic TDA-6®, and Juniper oil.

Another embodiment comprises cyclosporin A, Surfonic TDA-6/88®, and Juniper oil.

Another embodiment comprises cyclosporin A, Surfonic TDA-8®, and Juniper oil.

Another embodiment comprises cyclosporin A, Surfonic TDA-8/90®, and Juniper oil.

Another embodiment comprises cyclosporin A, Surfonic TDA-9®, and Juniper oil.

Another embodiment comprises cyclosporin A, Surfonic TX-CCN®, and Juniper oil.

Another embodiment comprises cyclosporin A, Tagat CH 40®, and Juniper oil.

Another embodiment comprises cyclosporin A, Tagat CH 60®, and Juniper oil.

Another embodiment comprises cyclosporin A, Tagat L 2®, and Juniper oil.

Another embodiment comprises cyclosporin A, Tagat S®, and Juniper oil.

Another embodiment comprises cyclosporin A, Tagat S 2®, and Juniper oil.

Another embodiment comprises cyclosporin A, Tally 100 Plus®, and Juniper oil.

Another embodiment comprises cyclosporin A, T-Det A106®, and Juniper oil.

Another embodiment comprises cyclosporin A, T-Det A109®, and Juniper oil.

Another embodiment comprises cyclosporin A, T-Det A136®, and Juniper oil.

Another embodiment comprises cyclosporin A, T-Det A139®, and Juniper oil.

Another embodiment comprises cyclosporin A, T-Det A2412®, and Juniper oil.

Another embodiment comprises cyclosporin A, T-Det A243®, and Juniper oil.

Another embodiment comprises cyclosporin A, T-Det A247®, and Juniper oil.

Another embodiment comprises cyclosporin A, T-Det A249®, and Juniper oil.

Another embodiment comprises cyclosporin A, T-Det A467®, and Juniper oil.

Another embodiment comprises cyclosporin A, T-Det COE®, and Juniper oil.

Another embodiment comprises cyclosporin A, T-Det DD-10®, and Juniper oil.

Another embodiment comprises cyclosporin A, T-Det DD-5®, and Juniper oil.

Another embodiment comprises cyclosporin A, T-Det DD-7®, and Juniper oil.

Another embodiment comprises cyclosporin A, T-Det EPO-104®, and Juniper oil.

Another embodiment comprises cyclosporin A, T-Det EPO-61®, and Juniper oil.

Another embodiment comprises cyclosporin A, T-Det EPO-62®, and Juniper oil.

Another embodiment comprises cyclosporin A, T-Det EPO-64®, and Juniper oil.

Another embodiment comprises cyclosporin A, T-Det N-1.5®, and Juniper oil.

Another embodiment comprises cyclosporin A, T-Det N-10.5®, and Juniper oil.

Another embodiment comprises cyclosporin A, T-Det N-100®, and Juniper oil.

Another embodiment comprises cyclosporin A, T-Det N-1007®, and Juniper oil.

Another embodiment comprises cyclosporin A, T-Det N-12®, and Juniper oil.

Another embodiment comprises cyclosporin A, T-Det N-14®, and Juniper oil.

Another embodiment comprises cyclosporin A, T-Det N-20®, and Juniper oil.

Another embodiment comprises cyclosporin A, T-Det N-30®, and Juniper oil.

Another embodiment comprises cyclosporin A, T-Det N-307®, and Juniper oil.

Another embodiment comprises cyclosporin A, T-Det N-4®, and Juniper oil.

Another embodiment comprises cyclosporin A, T-Det N-40®, and Juniper oil.

Another embodiment comprises cyclosporin A, T-Det N-407®, and Juniper oil.

Another embodiment comprises cyclosporin A, T-Det N-50®, and Juniper oil.

Another embodiment comprises cyclosporin A, T-Det N-507®, and Juniper oil.

Another embodiment comprises cyclosporin A, T-Det N-6®, and Juniper oil.

Another embodiment comprises cyclosporin A, T-Det N-70®, and Juniper oil.

Another embodiment comprises cyclosporin A, T-Det N-8®, and Juniper oil.

Another embodiment comprises cyclosporin A, T-Det N-9.5®, and Juniper oil.

Another embodiment comprises cyclosporin A, T-Det O-12®, and Juniper oil.

Another embodiment comprises cyclosporin A, T-Det O-165®, and Juniper oil.

Another embodiment comprises cyclosporin A, T-Det O-307®, and Juniper oil.

Another embodiment comprises cyclosporin A, T-Det O-40®, and Juniper oil.

Another embodiment comprises cyclosporin A, T-Det O-407®, and Juniper oil.

Another embodiment comprises cyclosporin A, T-Det O-6®, and Juniper oil.

Another embodiment comprises cyclosporin A, T-Det O-8®, and Juniper oil.

Another embodiment comprises cyclosporin A, T-Det O-9®, and Juniper oil.

Another embodiment comprises cyclosporin A, Tegin®, and Juniper oil.

Another embodiment comprises cyclosporin A, Tergitol 15-S-12®, and Juniper oil.

Another embodiment comprises cyclosporin A, Tergitol 15-S-15®, and Juniper oil.

Another embodiment comprises cyclosporin A, Tergitol 15-S-20®, and Juniper oil.

Another embodiment comprises cyclosporin A, Tergitol 15-S-20 (80AQ)®, and Juniper oil.

Another embodiment comprises cyclosporin A, Tergitol 15-S-3®, and Juniper oil.

Another embodiment comprises cyclosporin A, Tergitol 15-S-30®, and Juniper oil.

Another embodiment comprises cyclosporin A, Tergitol 15-S-40®, and Juniper oil.

Another embodiment comprises cyclosporin A, Tergitol 15-S-40 (70AQ)®, and Juniper oil.

Another embodiment comprises cyclosporin A, Tergitol 15-S-5®, and Juniper oil.

Another embodiment comprises cyclosporin A, Tergitol 15-S-7®, and Juniper oil.

Another embodiment comprises cyclosporin A, Tergitol 15-S-9®, and Juniper oil.

Another embodiment comprises cyclosporin A, Tergitol L-101®, and Juniper oil.

Another embodiment comprises cyclosporin A, Tergitol L-61®, and Juniper oil.

Another embodiment comprises cyclosporin A, Tergitol L-62®, and Juniper oil.

Another embodiment comprises cyclosporin A, Tergitol L-64®, and Juniper oil.

Another embodiment comprises cyclosporin A, Tergitol L-81®, and Juniper oil.

Another embodiment comprises cyclosporin A, Tergitol NP-10®, and Juniper oil.

Another embodiment comprises cyclosporin A, Tergitol NP-11®, and Juniper oil.

Another embodiment comprises cyclosporin A, Tergitol NP-12®, and Juniper oil.

Another embodiment comprises cyclosporin A, Tergitol NP-13®, and Juniper oil.

Another embodiment comprises cyclosporin A, Tergitol NP-15®, and Juniper oil.

Another embodiment comprises cyclosporin A, Tergitol NP-30®, and Juniper oil.

Another embodiment comprises cyclosporin A, Tergitol NP-30 (70 AQ)®, and Juniper oil.

Another embodiment comprises cyclosporin A, Tergitol NP-4®, and Juniper oil.

Another embodiment comprises cyclosporin A, Tergitol NP-40®, and Juniper oil.

Another embodiment comprises cyclosporin A, Tergitol NP-40 (70 AQ)®, and Juniper oil.

Another embodiment comprises cyclosporin A, Tergitol NP-50 (70 AQ)®, and Juniper oil.

Another embodiment comprises cyclosporin A, Tergitol NP-55 (70 AQ)®, and Juniper oil.

Another embodiment comprises cyclosporin A, Tergitol NP-6®, and Juniper oil.

Another embodiment comprises cyclosporin A, Tergitol NP-7®, and Juniper oil.

Another embodiment comprises cyclosporin A, Tergitol NP-70 (70 AQ)®, and Juniper oil.

Another embodiment comprises cyclosporin A, Tergitol NP-8®, and Juniper oil.

Another embodiment comprises cyclosporin A, Tergitol NP-9®, and Juniper oil.

Another embodiment comprises cyclosporin A, Tergitol NP-9.5®, and Juniper oil.

Another embodiment comprises cyclosporin A, Tergitol TMN-10®, and Juniper oil.

Another embodiment comprises cyclosporin A, Tergitol TMN-3®, and Juniper oil.

Another embodiment comprises cyclosporin A, Tergitol TMN-6®, and Juniper oil.

Another embodiment comprises cyclosporin A, Tergitol XD®, and Juniper oil.

Another embodiment comprises cyclosporin A, Tergitol XH®, and Juniper oil.

Another embodiment comprises cyclosporin A, Tergitol XJ®, and Juniper oil.

Another embodiment comprises cyclosporin A, Tetronic 1107®, and Juniper oil.

Another embodiment comprises cyclosporin A, Tetronic 1307®, and Juniper oil.

Another embodiment comprises cyclosporin A, Tetronic 150R1®, and Juniper oil.

Another embodiment comprises cyclosporin A, Tetronic 304®, and Juniper oil.

Another embodiment comprises cyclosporin A, Tetronic 701®, and Juniper oil.

Another embodiment comprises cyclosporin A, Tetronic 901®, and Juniper oil.

Another embodiment comprises cyclosporin A, Tetronic 904®, and Juniper oil.

Another embodiment comprises cyclosporin A, Tetronic 908®, and Juniper oil.

Another embodiment comprises cyclosporin A, Tetronic 90R4®, and Juniper oil.

Another embodiment comprises cyclosporin A, Tomadol 1-3®, and Juniper oil.

Another embodiment comprises cyclosporin A, Tomadol 1-5®, and Juniper oil.

Another embodiment comprises cyclosporin A, Tomadol 1-7®, and Juniper oil.

Another embodiment comprises cyclosporin A, Tomadol 23-1®, and Juniper oil.

Another embodiment comprises cyclosporin A, Tomadol 23-3®, and Juniper oil.

Another embodiment comprises cyclosporin A, Tomadol 23-5®, and Juniper oil.

Another embodiment comprises cyclosporin A, Tomadol 23-6.5®, and Juniper oil.

Another embodiment comprises cyclosporin A, Tomadol 25-12®, and Juniper oil.

Another embodiment comprises cyclosporin A, Tomadol 25-3®, and Juniper oil.

Another embodiment comprises cyclosporin A, Tomadol 25-7®, and Juniper oil.

Another embodiment comprises cyclosporin A, Tomadol 25-9®, and Juniper oil.

Another embodiment comprises cyclosporin A, Tomadol 45-13®, and Juniper oil.

Another embodiment comprises cyclosporin A, Tomadol 45-2.5®, and Juniper oil.

Another embodiment comprises cyclosporin A, Tomadol 45-7®, and Juniper oil.

Another embodiment comprises cyclosporin A, Tomadol 91-2.5®, and Juniper oil.

Another embodiment comprises cyclosporin A, Tomadol 91-6®, and Juniper oil.

Another embodiment comprises cyclosporin A, Tomadol 91-8®, and Juniper oil.

Another embodiment comprises cyclosporin A, Triton X-100®, and Juniper oil.

Another embodiment comprises cyclosporin A, Triton X-102®, and Juniper oil.

Another embodiment comprises cyclosporin A, Triton X-114®, and Juniper oil.

Another embodiment comprises cyclosporin A, Triton X-15®, and Juniper oil.

Another embodiment comprises cyclosporin A, Triton X-165 (70%)®, and Juniper oil.

Another embodiment comprises cyclosporin A, Triton X-207®, and Juniper oil.

Another embodiment comprises cyclosporin A, Triton X-305 (70%)®, and Juniper oil.

Another embodiment comprises cyclosporin A, Triton X-35®, and Juniper oil.

Another embodiment comprises cyclosporin A, Triton X-405 (70%)®, and Juniper oil.

Another embodiment comprises cyclosporin A, Triton X-45®, and Juniper oil.

Another embodiment comprises cyclosporin A, Triton X-705 (70%)®, and Juniper oil.

Another embodiment comprises cyclosporin A, Volpo S-10®, and Juniper oil.

Another embodiment comprises cyclosporin A, Volpo S-2®, and Juniper oil.

Another embodiment comprises cyclosporin A, Volpo S-20®, and Juniper oil.

Another embodiment comprises cyclosporin A, Volpo-10®, and Juniper oil.

Another embodiment comprises cyclosporin A, Volpo-20®, and Juniper oil.

Another embodiment comprises cyclosporin A, Volpo-3®, and Juniper oil.

Another embodiment comprises cyclosporin A, Volpo-5®, and Juniper oil.

Another embodiment comprises cyclosporin A, Abex 2515®, and Lemon oil.

Another embodiment comprises cyclosporin A, Abex 2525/40®, and Lemon oil.

Another embodiment comprises cyclosporin A, Abex 2535®, and Lemon oil.

Another embodiment comprises cyclosporin A, Abex 2545®, and Lemon oil.

Another embodiment comprises cyclosporin A, Acconon E®, and Lemon oil.

Another embodiment comprises cyclosporin A, Alfonic 1216CO-9®, and Lemon oil.

Another embodiment comprises cyclosporin A, Alfonic 1412-3®, and Lemon oil.

Another embodiment comprises cyclosporin A, Alfonic TDA-12®, and Lemon oil.

Another embodiment comprises cyclosporin A, Alfonic TDA-3®, and Lemon oil.

Another embodiment comprises cyclosporin A, Alfonic TDA-4®, and Lemon oil.

Another embodiment comprises cyclosporin A, Alfonic TDA-6®, and Lemon oil.

Another embodiment comprises cyclosporin A, Alfonic TDA-7®, and Lemon oil.

Another embodiment comprises cyclosporin A, Alfonic TDA-8®, and Lemon oil.

Another embodiment comprises cyclosporin A, Alfonic TDA-9®, and Lemon oil.

Another embodiment comprises cyclosporin A, Alkamuls 400-DO®, and Lemon oil.

Another embodiment comprises cyclosporin A, Alkamuls 400-MO/E®, and Lemon oil.

Another embodiment comprises cyclosporin A, Ameroxol OE-10®, and Lemon oil.

Another embodiment comprises cyclosporin A, Ameroxol OE-2®, and Lemon oil.

Another embodiment comprises cyclosporin A, Ameroxol OE-20®, and Lemon oil.

Another embodiment comprises cyclosporin A, Armix 176®, and Lemon oil.

Another embodiment comprises cyclosporin A, Armix 180-C®, and Lemon oil.

Another embodiment comprises cyclosporin A, Armix 183®, and Lemon oil.

Another embodiment comprises cyclosporin A, Brij 35®, and Lemon oil.

Another embodiment comprises cyclosporin A, Brij 35 Liquid®, and Lemon oil.

Another embodiment comprises cyclosporin A, Brij 35 SP®, and Lemon oil.

Another embodiment comprises cyclosporin A, Canasol MJ 2109®, and Lemon oil.

Another embodiment comprises cyclosporin A, Canasol MJ 52®, and Lemon oil.

Another embodiment comprises cyclosporin A, Canasol R 3600®, and Lemon oil.

Another embodiment comprises cyclosporin A, Canasol R 3603®, and Lemon oil.

Another embodiment comprises cyclosporin A, Canasol R 4000 H®, and Lemon oil.

Another embodiment comprises cyclosporin A, Cedepal CA-210®, and Lemon oil.

Another embodiment comprises cyclosporin A, Cedepal CA-520®, and Lemon oil.

Another embodiment comprises cyclosporin A, Cedepal CA-630®, and Lemon oil.

Another embodiment comprises cyclosporin A, Cedepal CA-720®, and Lemon oil.

Another embodiment comprises cyclosporin A, Cedepal CA-890®, and Lemon oil.

Another embodiment comprises cyclosporin A, Cedepal CA-897®, and Lemon oil.

Another embodiment comprises cyclosporin A, Cedepal CO-210®, and Lemon oil.

Another embodiment comprises cyclosporin A, Cedepal CO-430®, and Lemon oil.

Another embodiment comprises cyclosporin A, Cedepal CO-530®, and Lemon oil.

Another embodiment comprises cyclosporin A, Cedepal CO-630®, and Lemon oil.

Another embodiment comprises cyclosporin A, Cedepal CO-710®, and Lemon oil.

Another embodiment comprises cyclosporin A, Cedepal CO-730®, and Lemon oil.

Another embodiment comprises cyclosporin A, Cedepal CO-880®, and Lemon oil.

Another embodiment comprises cyclosporin A, Cedepal CO-887®, and Lemon oil.

Another embodiment comprises cyclosporin A, Cedepal CO-890®, and Lemon oil.

Another embodiment comprises cyclosporin A, Cedepal CO-897®, and Lemon oil.

Another embodiment comprises cyclosporin A, Cedepal CO-970®, and Lemon oil.

Another embodiment comprises cyclosporin A, Cedepal CO-977®, and Lemon oil.

Another embodiment comprises cyclosporin A, Cedepal CO-990®, and Lemon oil.

Another embodiment comprises cyclosporin A, Cedepal CO-997®, and Lemon oil.

Another embodiment comprises cyclosporin A, Chemal BP 261®, and Lemon oil.

Another embodiment comprises cyclosporin A, Chemal BP 262®, and Lemon oil.

Another embodiment comprises cyclosporin A, Chemal BP 3172®, and Lemon oil.

Another embodiment comprises cyclosporin A, Chemal BP 3174®, and Lemon oil.

Another embodiment comprises cyclosporin A, Chemal DA-4®, and Lemon oil.

Another embodiment comprises cyclosporin A, Chemal DA-6®, and Lemon oil.

Another embodiment comprises cyclosporin A, Chemal DA-9®, and Lemon oil.

Another embodiment comprises cyclosporin A, Chemal LA-23®, and Lemon oil.

Another embodiment comprises cyclosporin A, Chemal LA-4®, and Lemon oil.

Another embodiment comprises cyclosporin A, Chemal LA-9®, and Lemon oil.

Another embodiment comprises cyclosporin A, Chemal OA-20G®, and Lemon oil.

Another embodiment comprises cyclosporin A, Chemal OA-5®, and Lemon oil.

Another embodiment comprises cyclosporin A, Chemal OA-9®, and Lemon oil.

Another embodiment comprises cyclosporin A, Chemal TDA-12®, and Lemon oil.

Another embodiment comprises cyclosporin A, Chemal TDA-15®, and Lemon oil.

Another embodiment comprises cyclosporin A, Chemal TDA-3®, and Lemon oil.

Another embodiment comprises cyclosporin A, Chemal TDA-6®, and Lemon oil.

Another embodiment comprises cyclosporin A, Chemal TDA-9®, and Lemon oil.

Another embodiment comprises cyclosporin A, Chemax CO-16®, and Lemon oil.

Another embodiment comprises cyclosporin A, Chemax CO-200/50®, and Lemon oil.

Another embodiment comprises cyclosporin A, Chemax CO-25®, and Lemon oil.

Another embodiment comprises cyclosporin A, Chemax CO-30®, and Lemon oil.

Another embodiment comprises cyclosporin A, Chemax CO-36®, and Lemon oil.

Another embodiment comprises cyclosporin A, Chemax CO-40®, and Lemon oil.

Another embodiment comprises cyclosporin A, Chemax CO-5®, and Lemon oil.

Another embodiment comprises cyclosporin A, Chemax CO-80®, and Lemon oil.

Another embodiment comprises cyclosporin A, Chemax DNP-150/50®, and Lemon oil.

Another embodiment comprises cyclosporin A, Chemax DNP-18®, and Lemon oil.

Another embodiment comprises cyclosporin A, Chemax DNP-8®, and Lemon oil.

Another embodiment comprises cyclosporin A, Chemax E 200 ML®, and Lemon oil.

Another embodiment comprises cyclosporin A, Chemax E 200 MO®, and Lemon oil.

Another embodiment comprises cyclosporin A, Chemax E 400 ML®, and Lemon oil.

Another embodiment comprises cyclosporin A, Chemax E 400 MO®, and Lemon oil.

Another embodiment comprises cyclosporin A, Chemax E 600 ML®, and Lemon oil.

Another embodiment comprises cyclosporin A, Chemax E 600 MO®, and Lemon oil.

Another embodiment comprises cyclosporin A, Chemax E 1000 MO®, and Lemon oil.

Another embodiment comprises cyclosporin A, Chemax E 1000 MS®, and Lemon oil.

Another embodiment comprises cyclosporin A, Chemax E200 MS®, and Lemon oil.

Another embodiment comprises cyclosporin A, Chemax E400 MS®, and Lemon oil.

Another embodiment comprises cyclosporin A, Chemax E600 MS®, and Lemon oil.

Another embodiment comprises cyclosporin A, Chemax HCO-16®, and Lemon oil.

Another embodiment comprises cyclosporin A, Chemax HCO-200/50®, and Lemon oil.

Another embodiment comprises cyclosporin A, Chemax HCO-25®, and Lemon oil.

Another embodiment comprises cyclosporin A, Chemax HCO-5®, and Lemon oil.

Another embodiment comprises cyclosporin A, Chemax TO-16®, and Lemon oil.

Another embodiment comprises cyclosporin A, Chemax TO-8®, and Lemon oil.

Another embodiment comprises cyclosporin A, Colalube 3409®, and Lemon oil.

Another embodiment comprises cyclosporin A, Colalube 3411®, and Lemon oil.

Another embodiment comprises cyclosporin A, Colalube 3413®, and Lemon oil.

Another embodiment comprises cyclosporin A, Colalube 3414®, and Lemon oil.

Another embodiment comprises cyclosporin A, Colalube 3415®, and Lemon oil.

Another embodiment comprises cyclosporin A, Colalube 3416®, and Lemon oil.

Another embodiment comprises cyclosporin A, Colalube 3417®, and Lemon oil.

Another embodiment comprises cyclosporin A, Delonic NPE-100®, and Lemon oil.

Another embodiment comprises cyclosporin A, Delonic NPE-30®, and Lemon oil.

Another embodiment comprises cyclosporin A, Delonic NPE-40®, and Lemon oil.

Another embodiment comprises cyclosporin A, Delonic OPE-10®, and Lemon oil.

Another embodiment comprises cyclosporin A, Delonic OPE-12®, and Lemon oil.

Another embodiment comprises cyclosporin A, Delonic OPE-30®, and Lemon oil.

Another embodiment comprises cyclosporin A, Delonic OPE-40®, and Lemon oil.

Another embodiment comprises cyclosporin A, Delonic OPE-5®, and Lemon oil.

Another embodiment comprises cyclosporin A, Delonic OPE-7.5®, and Lemon oil.

Another embodiment comprises cyclosporin A, DePeg 16-CO®, and Lemon oil.

Another embodiment comprises cyclosporin A, DePeg 25-CO®, and Lemon oil.

Another embodiment comprises cyclosporin A, DePeg 30-CO®, and Lemon oil.

Another embodiment comprises cyclosporin A, DePeg 40-CO®, and Lemon oil.

Another embodiment comprises cyclosporin A, DePeg 5-CO®, and Lemon oil.

Another embodiment comprises cyclosporin A, DePeg 80-CO®, and Lemon oil.

Another embodiment comprises cyclosporin A, DeSonic S-100®, and Lemon oil.

Another embodiment comprises cyclosporin A, DeSonic S-114®, and Lemon oil.

Another embodiment comprises cyclosporin A, DeSonic S-405®, and Lemon oil.

Another embodiment comprises cyclosporin A, DeSonic S-45®, and Lemon oil.

Another embodiment comprises cyclosporin A, DeSonic TDA-9®, and Lemon oil.

Another embodiment comprises cyclosporin A, DeThox Acid O-14®, and Lemon oil.

Another embodiment comprises cyclosporin A, DeThox Acid O-16®, and Lemon oil.

Another embodiment comprises cyclosporin A, DeThox Acid O-9®, and Lemon oil.

Another embodiment comprises cyclosporin A, DeThox Acid TO-14®, and Lemon oil.

Another embodiment comprises cyclosporin A, DeThox Acid TO-16.5®, and Lemon oil.

Another embodiment comprises cyclosporin A, DeThox Acid TO-8.5®, and Lemon oil.

Another embodiment comprises cyclosporin A, Dur-Em 114®, and Lemon oil.

Another embodiment comprises cyclosporin A, Dur-Em 117®, and Lemon oil.

Another embodiment comprises cyclosporin A, Dur-Em 204®, and Lemon oil.

Another embodiment comprises cyclosporin A, Dur-Em 207®, and Lemon oil.

Another embodiment comprises cyclosporin A, Dur-Em 207E®, and Lemon oil.

Another embodiment comprises cyclosporin A, Emerest 2704®, and Lemon oil.

Another embodiment comprises cyclosporin A, Emerest 2712®, and Lemon oil.

Another embodiment comprises cyclosporin A, Emerest 2715®, and Lemon oil.

Another embodiment comprises cyclosporin A, Ethal EH-2®, and Lemon oil.

Another embodiment comprises cyclosporin A, Ethal EH-5®, and Lemon oil.

Another embodiment comprises cyclosporin A, Ethal OA-23®, and Lemon oil.

Another embodiment comprises cyclosporin A, Ethosperse CA-20®, and Lemon oil.

Another embodiment comprises cyclosporin A, Ethosperse LA-12®, and Lemon oil.

Another embodiment comprises cyclosporin A, Ethosperse LA-23®, and Lemon oil.

Another embodiment comprises cyclosporin A, Ethosperse LA-4®, and Lemon oil.

Another embodiment comprises cyclosporin A, Ethox 3095®, and Lemon oil.

Another embodiment comprises cyclosporin A, Ethox CO-16®, and Lemon oil.

Another embodiment comprises cyclosporin A, Ethox CO-200®, and Lemon oil.

Another embodiment comprises cyclosporin A, Ethox CO-25®, and Lemon oil.

Another embodiment comprises cyclosporin A, Ethox CO-30®, and Lemon oil.

Another embodiment comprises cyclosporin A, Ethox CO-36®, and Lemon oil.

Another embodiment comprises cyclosporin A, Ethox CO-40®, and Lemon oil.

Another embodiment comprises cyclosporin A, Ethox CO-5®, and Lemon oil.

Another embodiment comprises cyclosporin A, Ethox DO-14®, and Lemon oil.

Another embodiment comprises cyclosporin A, Ethox DO-9®, and Lemon oil.

Another embodiment comprises cyclosporin A, Ethox MA-8®, and Lemon oil.

Another embodiment comprises cyclosporin A, Ethox MI-14®, and Lemon oil.

Another embodiment comprises cyclosporin A, Ethox ML-14®, and Lemon oil.

Another embodiment comprises cyclosporin A, Ethox ML-5®, and Lemon oil.

Another embodiment comprises cyclosporin A, Ethox MO-14®, and Lemon oil.

Another embodiment comprises cyclosporin A, Ethox MO-9®, and Lemon oil.

Another embodiment comprises cyclosporin A, Ethox MS-14®, and Lemon oil.

Another embodiment comprises cyclosporin A, Ethox MS-23®, and Lemon oil.

Another embodiment comprises cyclosporin A, Ethox MS-40®, and Lemon oil.

Another embodiment comprises cyclosporin A, Ethox MS-8®, and Lemon oil.

Another embodiment comprises cyclosporin A, Ethox TO-16®, and Lemon oil.

Another embodiment comprises cyclosporin A, Ethox TO-8®, and Lemon oil.

Another embodiment comprises cyclosporin A, Ethylan SN-120®, and Lemon oil.

Another embodiment comprises cyclosporin A, Ethylan SN-70®, and Lemon oil.

Another embodiment comprises cyclosporin A, Ethylan SN-90®, and Lemon oil.

Another embodiment comprises cyclosporin A, Flexricin 13®, and Lemon oil.

Another embodiment comprises cyclosporin A, Flexricin 15®, and Lemon oil.

Another embodiment comprises cyclosporin A, Genapol 1261®, and Lemon oil.

Another embodiment comprises cyclosporin A, Generol 122 E25®, and Lemon oil.

Another embodiment comprises cyclosporin A, Glicepol 160®, and Lemon oil.

Another embodiment comprises cyclosporin A, Glicepol 560®, and Lemon oil.

Another embodiment comprises cyclosporin A, Glicepol GMS 20®, and Lemon oil.

Another embodiment comprises cyclosporin A, Hetoxide BN-13®, and Lemon oil.

Another embodiment comprises cyclosporin A, Hetoxide DNP-4®, and Lemon oil.

Another embodiment comprises cyclosporin A, Hetoxide DNP-9.6®, and Lemon oil.

Another embodiment comprises cyclosporin A, Hetoxide G-26®, and Lemon oil.

Another embodiment comprises cyclosporin A, Hetoxide G-7®, and Lemon oil.

Another embodiment comprises cyclosporin A, Hetoxide GT-20®, and Lemon oil.

Another embodiment comprises cyclosporin A, Hetoxide HC-40®, and Lemon oil.

Another embodiment comprises cyclosporin A, Hetoxol C-24®, and Lemon oil.

Another embodiment comprises cyclosporin A, Hetoxol CA-10®, and Lemon oil.

Another embodiment comprises cyclosporin A, Hetoxol CA-2®, and Lemon oil.

Another embodiment comprises cyclosporin A, Hetoxol CA-20®, and Lemon oil.

Another embodiment comprises cyclosporin A, Hetoxol CAWS®, and Lemon oil.

Another embodiment comprises cyclosporin A, Hetoxol CD-3®, and Lemon oil.

Another embodiment comprises cyclosporin A, Hetoxol CD-4®, and Lemon oil.

Another embodiment comprises cyclosporin A, Hetoxol CS-15®, and Lemon oil.

Another embodiment comprises cyclosporin A, Hetoxol CS-20®, and Lemon oil.

Another embodiment comprises cyclosporin A, Hetoxol CS-20D®, and Lemon oil.

Another embodiment comprises cyclosporin A, Hetoxol CS-25®, and Lemon oil.

Another embodiment comprises cyclosporin A, Hetoxol CS-50®, and Lemon oil.

Another embodiment comprises cyclosporin A, Hetoxol CS-9®, and Lemon oil.

Another embodiment comprises cyclosporin A, Hetoxol CSA-15®, and Lemon oil.

Another embodiment comprises cyclosporin A, Hetoxol D®, and Lemon oil.

Another embodiment comprises cyclosporin A, Hetoxol G®, and Lemon oil.

Another embodiment comprises cyclosporin A, Hetoxol J®, and Lemon oil.

Another embodiment comprises cyclosporin A, Hetoxol L®, and Lemon oil.

Another embodiment comprises cyclosporin A, Hetoxol L-23®, and Lemon oil.

Another embodiment comprises cyclosporin A, Hetoxol L-4®, and Lemon oil.

Another embodiment comprises cyclosporin A, Hetoxol L-9®, and Lemon oil.

Another embodiment comprises cyclosporin A, Hetoxol LS-9®, and Lemon oil.

Another embodiment comprises cyclosporin A, Hetoxol M-3®, and Lemon oil.

Another embodiment comprises cyclosporin A, Hetoxol OA-10 Special®, and Lemon oil.

Another embodiment comprises cyclosporin A, Hetoxol OA-20 Special®, and Lemon oil.

Another embodiment comprises cyclosporin A, Hetoxol OA-3 Special®, and Lemon oil.

Another embodiment comprises cyclosporin A, Hetoxol OA-5 Special®, and Lemon oil.

Another embodiment comprises cyclosporin A, Hetoxol OL-10®, and Lemon oil.

Another embodiment comprises cyclosporin A, Hetoxol OL-2®, and Lemon oil.

Another embodiment comprises cyclosporin A, Hetoxol OL-23®, and Lemon oil.

Another embodiment comprises cyclosporin A, Hetoxol OL-4®, and Lemon oil.

Another embodiment comprises cyclosporin A, Hetoxol OL-40®, and Lemon oil.

Another embodiment comprises cyclosporin A, Hetoxol OL-5®, and Lemon oil.

Another embodiment comprises cyclosporin A, Hetoxol PLA®, and Lemon oil.

Another embodiment comprises cyclosporin A, Hetoxol SP-15®, and Lemon oil.

Another embodiment comprises cyclosporin A, Hetoxol STA-10®, and Lemon oil.

Another embodiment comprises cyclosporin A, Hetoxol STA-2®, and Lemon oil.

Another embodiment comprises cyclosporin A, Hetoxol STA-30®, and Lemon oil.

Another embodiment comprises cyclosporin A, Hetoxol TD-12®, and Lemon oil.

Another embodiment comprises cyclosporin A, Hetoxol TD-18®, and Lemon oil.

Another embodiment comprises cyclosporin A, Hetoxol TD-3®, and Lemon oil.

Another embodiment comprises cyclosporin A, Hetoxol TD-6®, and Lemon oil.

Another embodiment comprises cyclosporin A, Hetoxol TD-9®, and Lemon oil.

Another embodiment comprises cyclosporin A, Hetoxol TDEP-15®, and Lemon oil.

Another embodiment comprises cyclosporin A, Hetoxol TDEP-63®, and Lemon oil.

Another embodiment comprises cyclosporin A, Iconol 24-12®, and Lemon oil.

Another embodiment comprises cyclosporin A, Iconol 24-23®, and Lemon oil.

Another embodiment comprises cyclosporin A, Iconol 24-4®, and Lemon oil.

Another embodiment comprises cyclosporin A, Iconol DA-4®, and Lemon oil.

Another embodiment comprises cyclosporin A, Iconol DA-6®, and Lemon oil.

Another embodiment comprises cyclosporin A, Iconol TDA-10®, and Lemon oil.

Another embodiment comprises cyclosporin A, Iconol TDA-3®, and Lemon oil.

Another embodiment comprises cyclosporin A, Iconol TDA-6®, and Lemon oil.

Another embodiment comprises cyclosporin A, Iconol TDA-8-90%®, and Lemon oil.

Another embodiment comprises cyclosporin A, Iconol TDA-9®, and Lemon oil.

Another embodiment comprises cyclosporin A, Igepal CA-210®, and Lemon oil.

Another embodiment comprises cyclosporin A, Igepal CA-407®, and Lemon oil.

Another embodiment comprises cyclosporin A, Igepal CA-420®, and Lemon oil.

Another embodiment comprises cyclosporin A, Igepal CA-520®, and Lemon oil.

Another embodiment comprises cyclosporin A, Igepal CA-620®, and Lemon oil.

Another embodiment comprises cyclosporin A, Igepal CA-630®, and Lemon oil.

Another embodiment comprises cyclosporin A, Igepal CA-720®, and Lemon oil.

Another embodiment comprises cyclosporin A, Igepal CA-877®, and Lemon oil.

Another embodiment comprises cyclosporin A, Igepal CA-887®, and Lemon oil.

Another embodiment comprises cyclosporin A, Igepal CA-897®, and Lemon oil.

Another embodiment comprises cyclosporin A, Igepal CO-210®, and Lemon oil.

Another embodiment comprises cyclosporin A, Igepal CO-430®, and Lemon oil.

Another embodiment comprises cyclosporin A, Igepal CO-520®, and Lemon oil.

Another embodiment comprises cyclosporin A, Igepal CO-530®, and Lemon oil.

Another embodiment comprises cyclosporin A, Igepal CO-610®, and Lemon oil.

Another embodiment comprises cyclosporin A, Igepal CO-630®, and Lemon oil.

Another embodiment comprises cyclosporin A, Igepal CO-630 Special®, and Lemon oil.

Another embodiment comprises cyclosporin A, Igepal CO-660®, and Lemon oil.

Another embodiment comprises cyclosporin A, Igepal CO-710®, and Lemon oil.

Another embodiment comprises cyclosporin A, Igepal CO-720®, and Lemon oil.

Another embodiment comprises cyclosporin A, Igepal CO-730®, and Lemon oil.

Another embodiment comprises cyclosporin A, Igepal CO-738®, and Lemon oil.

Another embodiment comprises cyclosporin A, Igepal CO-850®, and Lemon oil.

Another embodiment comprises cyclosporin A, Igepal CO-880®, and Lemon oil.

Another embodiment comprises cyclosporin A, Igepal CO-887®, and Lemon oil.

Another embodiment comprises cyclosporin A, Igepal CO-890®, and Lemon oil.

Another embodiment comprises cyclosporin A, Igepal CO-897®, and Lemon oil.

Another embodiment comprises cyclosporin A, Igepal CO-970®, and Lemon oil.

Another embodiment comprises cyclosporin A, Igepal CO-977®, and Lemon oil.

Another embodiment comprises cyclosporin A, Igepal CO-987®, and Lemon oil.

Another embodiment comprises cyclosporin A, Igepal CO-990 FLK®, and Lemon oil.

Another embodiment comprises cyclosporin A, Igepal CO-997®, and Lemon oil.

Another embodiment comprises cyclosporin A, Igepal DM-430®, and Lemon oil.

Another embodiment comprises cyclosporin A, Igepal DM-530®, and Lemon oil.

Another embodiment comprises cyclosporin A, Igepal DM-710®, and Lemon oil.

Another embodiment comprises cyclosporin A, Igepal DM-730®, and Lemon oil.

Another embodiment comprises cyclosporin A, Igepal DM-880®, and Lemon oil.

Another embodiment comprises cyclosporin A, Igepal DM-970 FLK®, and Lemon oil.

Another embodiment comprises cyclosporin A, Igepal OD-410®, and Lemon oil.

Another embodiment comprises cyclosporin A, Igepal RC-520®, and Lemon oil.

Another embodiment comprises cyclosporin A, Igepal RC-620®, and Lemon oil.

Another embodiment comprises cyclosporin A, Igepal RC-630®, and Lemon oil.

Another embodiment comprises cyclosporin A, Lexemul CS-20®, and Lemon oil.

Another embodiment comprises cyclosporin A, Lipo GMS 450®, and Lemon oil.

Another embodiment comprises cyclosporin A, Lipo Polyglycol 1000®, and Lemon oil.

Another embodiment comprises cyclosporin A, Lipo Polyglycol 200®, and Lemon oil.

Another embodiment comprises cyclosporin A, Lipo Polyglycol 300®, and Lemon oil.

Another embodiment comprises cyclosporin A, Lipo Polyglycol 3350®, and Lemon oil.

Another embodiment comprises cyclosporin A, Lipo Polyglycol 400®, and Lemon oil.

Another embodiment comprises cyclosporin A, Lipo Polyglycol 600®, and Lemon oil.

Another embodiment comprises cyclosporin A, Lipocol C-10®, and Lemon oil.

Another embodiment comprises cyclosporin A, Lipocol C-2®, and Lemon oil.

Another embodiment comprises cyclosporin A, Lipocol C-20®, and Lemon oil.

Another embodiment comprises cyclosporin A, Lipocol L-12®, and Lemon oil.

Another embodiment comprises cyclosporin A, Lipocol L-23®, and Lemon oil.

Another embodiment comprises cyclosporin A, Lipocol L-4®, and Lemon oil.

Another embodiment comprises cyclosporin A, Lipocol O-10®, and Lemon oil.

Another embodiment comprises cyclosporin A, Lipocol O-2®, and Lemon oil.

Another embodiment comprises cyclosporin A, Lipocol O-20®, and Lemon oil.

Another embodiment comprises cyclosporin A, Lipocol O-5®, and Lemon oil.

Another embodiment comprises cyclosporin A, Lipocol S-10®, and Lemon oil.

Another embodiment comprises cyclosporin A, Lipocol S-2®, and Lemon oil.

Another embodiment comprises cyclosporin A, Lipocol S-20®, and Lemon oil.

Another embodiment comprises cyclosporin A, Lipocol SC-15®, and Lemon oil.

Another embodiment comprises cyclosporin A, Lipocol SC-20®, and Lemon oil.

Another embodiment comprises cyclosporin A, Lipocol SC-4®, and Lemon oil.

Another embodiment comprises cyclosporin A, Lipocol TD-12®, and Lemon oil.

Another embodiment comprises cyclosporin A, Lumulse 40-O K®, and Lemon oil.

Another embodiment comprises cyclosporin A, Lumulse 42-O K®, and Lemon oil.

Another embodiment comprises cyclosporin A, Lumulse GMT-40®, and Lemon oil.

Another embodiment comprises cyclosporin A, Lumulse PEG 1450®, and Lemon oil.

Another embodiment comprises cyclosporin A, Lumulse PEG 1450 NF®, and Lemon oil.

Another embodiment comprises cyclosporin A, Lumulse PEG 8000®, and Lemon oil.

Another embodiment comprises cyclosporin A, Lumulse PGO®, and Lemon oil.

Another embodiment comprises cyclosporin A, Lumulse POE (2) Oleyl Amine®, and Lemon oil.

Another embodiment comprises cyclosporin A, Magrabar PGO-104®, and Lemon oil.

Another embodiment comprises cyclosporin A, Mapeg 200 ML®, and Lemon oil.

Another embodiment comprises cyclosporin A, Mapeg 400 MO®, and Lemon oil.

Another embodiment comprises cyclosporin A, Mapeg 400 MOT®, and Lemon oil.

Another embodiment comprises cyclosporin A, Mapeg 400DO®, and Lemon oil.

Another embodiment comprises cyclosporin A, Mapeg 400DOT®, and Lemon oil.

Another embodiment comprises cyclosporin A, Mapeg 600DOT®, and Lemon oil.

Another embodiment comprises cyclosporin A, Marlox B 24/80®, and Lemon oil.

Another embodiment comprises cyclosporin A, Marlox FK 86®, and Lemon oil.

Another embodiment comprises cyclosporin A, Marlox MO 154®, and Lemon oil.

Another embodiment comprises cyclosporin A, Mazox CDA CG®, and Lemon oil.

Another embodiment comprises cyclosporin A, Monoglycerides Glyceryl Monestearate®, and Lemon oil.

Another embodiment comprises cyclosporin A, Myrj 45®, and Lemon oil.

Another embodiment comprises cyclosporin A, Myrj 52®, and Lemon oil.

Another embodiment comprises cyclosporin A, Myrj 52S®, and Lemon oil.

Another embodiment comprises cyclosporin A, Myrj 53®, and Lemon oil.

Another embodiment comprises cyclosporin A, Myrj 59®, and Lemon oil.

Another embodiment comprises cyclosporin A, Norfox NP-12®, and Lemon oil.

Another embodiment comprises cyclosporin A, Norfox NP-4®, and Lemon oil.

Another embodiment comprises cyclosporin A, Norfox NP-6®, and Lemon oil.

Another embodiment comprises cyclosporin A, Norfox NP-887®, and Lemon oil.

Another embodiment comprises cyclosporin A, Norfox NP-9®, and Lemon oil.

Another embodiment comprises cyclosporin A, Norfox NP-977®, and Lemon oil.

Another embodiment comprises cyclosporin A, Norfox OP-100®, and Lemon oil.

Another embodiment comprises cyclosporin A, Norfox OP-102®, and Lemon oil.

Another embodiment comprises cyclosporin A, Norfox OP-114®, and Lemon oil.

Another embodiment comprises cyclosporin A, Norfox OP-45®, and Lemon oil.

Another embodiment comprises cyclosporin A, Pegosperse 100-L®, and Lemon oil.

Another embodiment comprises cyclosporin A, Pegosperse 100-O®, and Lemon oil.

Another embodiment comprises cyclosporin A, Pegosperse 100-S®, and Lemon oil.

Another embodiment comprises cyclosporin A, Pegosperse 1500-MS®, and Lemon oil.

Another embodiment comprises cyclosporin A, Pegosperse 1750-MS®, and Lemon oil.

Another embodiment comprises cyclosporin A, Pegosperse 200 DL®, and Lemon oil.

Another embodiment comprises cyclosporin A, Pegosperse 200-ML®, and Lemon oil.

Another embodiment comprises cyclosporin A, Pegosperse 400-DL®, and Lemon oil.

Another embodiment comprises cyclosporin A, Pegosperse 400-DO®, and Lemon oil.

Another embodiment comprises cyclosporin A, Pegosperse 400-DS®, and Lemon oil.

Another embodiment comprises cyclosporin A, Pegosperse 400-ML®, and Lemon oil.

Another embodiment comprises cyclosporin A, Pegosperse 400-MO®, and Lemon oil.

Another embodiment comprises cyclosporin A, Pegosperse 400-MOT®, and Lemon oil.

Another embodiment comprises cyclosporin A, Pegosperse 400-MS®, and Lemon oil.

Another embodiment comprises cyclosporin A, Pegosperse 50-DS®, and Lemon oil.

Another embodiment comprises cyclosporin A, Pegosperse 50-MS®, and Lemon oil.

Another embodiment comprises cyclosporin A, Pegosperse 600-ML®, and Lemon oil.

Another embodiment comprises cyclosporin A, Pegosperse 600-MS®, and Lemon oil.

Another embodiment comprises cyclosporin A, Plurafac A-38®, and Lemon oil.

Another embodiment comprises cyclosporin A, Plurafac B-25-5®, and Lemon oil.

Another embodiment comprises cyclosporin A, Plurafac B-26®, and Lemon oil.

Another embodiment comprises cyclosporin A, Plurafac D-25®, and Lemon oil.

Another embodiment comprises cyclosporin A, Plurafac LF 131®, and Lemon oil.

Another embodiment comprises cyclosporin A, Plurafac LF 4030®, and Lemon oil.

Another embodiment comprises cyclosporin A, Plurafac LF 7000®, and Lemon oil.

Another embodiment comprises cyclosporin A, Plurafac RA-20®, and Lemon oil.

Another embodiment comprises cyclosporin A, Plurafac RA-30®, and Lemon oil.

Another embodiment comprises cyclosporin A, Plurafac RA-40®, and Lemon oil.

Another embodiment comprises cyclosporin A, Pluronic 10R5®, and Lemon oil.

Another embodiment comprises cyclosporin A, Pluronic 17R2®, and Lemon oil.

Another embodiment comprises cyclosporin A, Pluronic 17R4®, and Lemon oil.

Another embodiment comprises cyclosporin A, Pluronic 25R2®, and Lemon oil.

Another embodiment comprises cyclosporin A, Pluronic 25R4®, and Lemon oil.

Another embodiment comprises cyclosporin A, Pluronic 31R1®, and Lemon oil.

Another embodiment comprises cyclosporin A, Pluronic F108®, and Lemon oil.

Another embodiment comprises cyclosporin A, Pluronic F127®, and Lemon oil.

Another embodiment comprises cyclosporin A, Pluronic F38®, and Lemon oil.

Another embodiment comprises cyclosporin A, Pluronic F68®, and Lemon oil.

Another embodiment comprises cyclosporin A, Pluronic F68LF®, and Lemon oil.

Another embodiment comprises cyclosporin A, Pluronic F77®, and Lemon oil.

Another embodiment comprises cyclosporin A, Pluronic F87®, and Lemon oil.

Another embodiment comprises cyclosporin A, Pluronic F88®, and Lemon oil.

Another embodiment comprises cyclosporin A, Pluronic F98®, and Lemon oil.

Another embodiment comprises cyclosporin A, Pluronic L10®, and Lemon oil.

Another embodiment comprises cyclosporin A, Pluronic L101®, and Lemon oil.

Another embodiment comprises cyclosporin A, Pluronic L121®, and Lemon oil.

Another embodiment comprises cyclosporin A, Pluronic L31®, and Lemon oil.

Another embodiment comprises cyclosporin A, Pluronic L35®, and Lemon oil.

Another embodiment comprises cyclosporin A, Pluronic L43®, and Lemon oil.

Another embodiment comprises cyclosporin A, Pluronic L44®, and Lemon oil.

Another embodiment comprises cyclosporin A, Pluronic L61®, and Lemon oil.

Another embodiment comprises cyclosporin A, Pluronic L62®, and Lemon oil.

Another embodiment comprises cyclosporin A, Pluronic L62D®, and Lemon oil.

Another embodiment comprises cyclosporin A, Pluronic L62LF®, and Lemon oil.

Another embodiment comprises cyclosporin A, Pluronic L64®, and Lemon oil.

Another embodiment comprises cyclosporin A, Pluronic L81®, and Lemon oil.

Another embodiment comprises cyclosporin A, Pluronic L92®, and Lemon oil.

Another embodiment comprises cyclosporin A, Pluronic P103®, and Lemon oil.

Another embodiment comprises cyclosporin A, Pluronic P104®, and Lemon oil.

Another embodiment comprises cyclosporin A, Pluronic P105®, and Lemon oil.

Another embodiment comprises cyclosporin A, Pluronic P123®, and Lemon oil.

Another embodiment comprises cyclosporin A, Pluronic P65®, and Lemon oil.

Another embodiment comprises cyclosporin A, Pluronic P84®, and Lemon oil.

Another embodiment comprises cyclosporin A, Pluronic P85®, and Lemon oil.

Another embodiment comprises cyclosporin A, Polyaldo 10-2-P®, and Lemon oil.

Another embodiment comprises cyclosporin A, Polyaldo DGDO®, and Lemon oil.

Another embodiment comprises cyclosporin A, Polyaldo HGDS®, and Lemon oil.

Another embodiment comprises cyclosporin A, Polyaldo TGMS®, and Lemon oil.

Another embodiment comprises cyclosporin A, Protegin V®, and Lemon oil.

Another embodiment comprises cyclosporin A, Protegin W®, and Lemon oil.

Another embodiment comprises cyclosporin A, Protegin WX®, and Lemon oil.

Another embodiment comprises cyclosporin A, Protegin XV®, and Lemon oil.

Another embodiment comprises cyclosporin A, Rhodasurf BC-420®, and Lemon oil.

Another embodiment comprises cyclosporin A, Rhodasurf BC-610®, and Lemon oil.

Another embodiment comprises cyclosporin A, Rhodasurf BC-630®, and Lemon oil.

Another embodiment comprises cyclosporin A, Rhodasurf BC-720®, and Lemon oil.

Another embodiment comprises cyclosporin A, Rhodasurf BC-840®, and Lemon oil.

Another embodiment comprises cyclosporin A, Rhodasurf L 1®, and Lemon oil.

Another embodiment comprises cyclosporin A, Rhodasurf L 12®, and Lemon oil.

Another embodiment comprises cyclosporin A, Rhodasurf L 2®, and Lemon oil.

Another embodiment comprises cyclosporin A, Rhodasurf L 20®, and Lemon oil.

Another embodiment comprises cyclosporin A, Rhodasurf L 3®, and Lemon oil.

Another embodiment comprises cyclosporin A, Rhodasurf L 30®, and Lemon oil.

Another embodiment comprises cyclosporin A, Rhodasurf L 4®, and Lemon oil.

Another embodiment comprises cyclosporin A, Rhodasurf L 50®, and Lemon oil.

Another embodiment comprises cyclosporin A, Rhodasurf L 7/90®, and Lemon oil.

Another embodiment comprises cyclosporin A, Rhodasurf LA-3®, and Lemon oil.

Another embodiment comprises cyclosporin A, Rhodasurf LA-7®, and Lemon oil.

Another embodiment comprises cyclosporin A, Rhodasurf LAN-23®, and Lemon oil.

Another embodiment comprises cyclosporin A, Rhodasurf ON-870®, and Lemon oil.

Another embodiment comprises cyclosporin A, Rhodasurf ON-877®, and Lemon oil.

Another embodiment comprises cyclosporin A, Rhodasurf TDA-8/5®, and Lemon oil.

Another embodiment comprises cyclosporin A, Ritapro 100®, and Lemon oil.

Another embodiment comprises cyclosporin A, Ritapro 165®, and Lemon oil.

Another embodiment comprises cyclosporin A, Ritapro 200®, and Lemon oil.

Another embodiment comprises cyclosporin A, Ritapro 300®, and Lemon oil.

Another embodiment comprises cyclosporin A, Ritoleth 10®, and Lemon oil.

Another embodiment comprises cyclosporin A, Ritoleth 2®, and Lemon oil.

Another embodiment comprises cyclosporin A, Ritoleth 20®, and Lemon oil.

Another embodiment comprises cyclosporin A, Ritoleth 5®, and Lemon oil.

Another embodiment comprises cyclosporin A, Santone 10-10-0®, and Lemon oil.

Another embodiment comprises cyclosporin A, Santone 3-1-S XTR®, and Lemon oil.

Another embodiment comprises cyclosporin A, Santone 3-1-SH®, and Lemon oil.

Another embodiment comprises cyclosporin A, Santone 8-1-O®, and Lemon oil.

Another embodiment comprises cyclosporin A, Solulan 16®, and Lemon oil.

Another embodiment comprises cyclosporin A, Solulan 75®, and Lemon oil.

Another embodiment comprises cyclosporin A, Solulan C-24®, and Lemon oil.

Another embodiment comprises cyclosporin A, Solulan L-575®, and Lemon oil.

Another embodiment comprises cyclosporin A, Standamox CAW®, and Lemon oil.

Another embodiment comprises cyclosporin A, Sunine AZI-A®, and Lemon oil.

Another embodiment comprises cyclosporin A, Surfonic CO-15®, and Lemon oil.

Another embodiment comprises cyclosporin A, Surfonic CO-25®, and Lemon oil.

Another embodiment comprises cyclosporin A, Surfonic CO-30®, and Lemon oil.

Another embodiment comprises cyclosporin A, Surfonic CO-36®, and Lemon oil.

Another embodiment comprises cyclosporin A, Surfonic CO-42®, and Lemon oil.

Another embodiment comprises cyclosporin A, Surfonic E400-MO®, and Lemon oil.

Another embodiment comprises cyclosporin A, Surfonic L12-3®, and Lemon oil.

Another embodiment comprises cyclosporin A, Surfonic L12-6®, and Lemon oil.

Another embodiment comprises cyclosporin A, Surfonic L12-8®, and Lemon oil.

Another embodiment comprises cyclosporin A, Surfonic L24-12®, and Lemon oil.

Another embodiment comprises cyclosporin A, Surfonic L24-2®, and Lemon oil.

Another embodiment comprises cyclosporin A, Surfonic L24-22®, and Lemon oil.

Another embodiment comprises cyclosporin A, Surfonic L24-3®, and Lemon oil.

Another embodiment comprises cyclosporin A, Surfonic L24-4®, and Lemon oil.

Another embodiment comprises cyclosporin A, Surfonic L24-5®, and Lemon oil.

Another embodiment comprises cyclosporin A, Surfonic L24-7®, and Lemon oil.

Another embodiment comprises cyclosporin A, Surfonic L24-7.1®, and Lemon oil.

Another embodiment comprises cyclosporin A, Surfonic L24-9®, and Lemon oil.

Another embodiment comprises cyclosporin A, Surfonic LF-17®, and Lemon oil.

Another embodiment comprises cyclosporin A, Surfonic LF-18®, and Lemon oil.

Another embodiment comprises cyclosporin A, Surfonic LF-37®, and Lemon oil.

Another embodiment comprises cyclosporin A, Surfonic LF-41®, and Lemon oil.

Another embodiment comprises cyclosporin A, Surfonic LF-42®, and Lemon oil.

Another embodiment comprises cyclosporin A, Surfonic LF-47®, and Lemon oil.

Another embodiment comprises cyclosporin A, Surfonic LF-68®, and Lemon oil.

Another embodiment comprises cyclosporin A, Surfonic N-10®, and Lemon oil.

Another embodiment comprises cyclosporin A, Surfonic N-100®, and Lemon oil.

Another embodiment comprises cyclosporin A, Surfonic N-102®, and Lemon oil.

Another embodiment comprises cyclosporin A, Surfonic N-120®, and Lemon oil.

Another embodiment comprises cyclosporin A, Surfonic N-150®, and Lemon oil.

Another embodiment comprises cyclosporin A, Surfonic N-200®, and Lemon oil.

Another embodiment comprises cyclosporin A, Surfonic N-300®, and Lemon oil.

Another embodiment comprises cyclosporin A, Surfonic N-31.5®, and Lemon oil.

Another embodiment comprises cyclosporin A, Surfonic N-40®, and Lemon oil.

Another embodiment comprises cyclosporin A, Surfonic N-400®, and Lemon oil.

Another embodiment comprises cyclosporin A, Surfonic N-60®, and Lemon oil.

Another embodiment comprises cyclosporin A, Surfonic N-85®, and Lemon oil.

Another embodiment comprises cyclosporin A, Surfonic N-95®, and Lemon oil.

Another embodiment comprises cyclosporin A, Surfonic NB-18®, and Lemon oil.

Another embodiment comprises cyclosporin A, Surfonic NB-23®, and Lemon oil.

Another embodiment comprises cyclosporin A, Surfonic NB-25®, and Lemon oil.

Another embodiment comprises cyclosporin A, Surfonic NB-307®, and Lemon oil.

Another embodiment comprises cyclosporin A, Surfonic NB-31®, and Lemon oil.

Another embodiment comprises cyclosporin A, Surfonic NB-41®, and Lemon oil.

Another embodiment comprises cyclosporin A, Surfonic NB-43®, and Lemon oil.

Another embodiment comprises cyclosporin A, Surfonic OP-100®, and Lemon oil.

Another embodiment comprises cyclosporin A, Surfonic OP-120®, and Lemon oil.

Another embodiment comprises cyclosporin A, Surfonic OP-15®, and Lemon oil.

Another embodiment comprises cyclosporin A, Surfonic OP-35®, and Lemon oil.

Another embodiment comprises cyclosporin A, Surfonic OP-70®, and Lemon oil.

Another embodiment comprises cyclosporin A, Surfonic P1®, and Lemon oil.

Another embodiment comprises cyclosporin A, Surfonic P3®, and Lemon oil.

Another embodiment comprises cyclosporin A, Surfonic P5®, and Lemon oil.

Another embodiment comprises cyclosporin A, Surfonic P6®, and Lemon oil.

Another embodiment comprises cyclosporin A, Surfonic POA-17R2®, and Lemon oil.

Another embodiment comprises cyclosporin A, Surfonic POA-25R2®, and Lemon oil.

Another embodiment comprises cyclosporin A, Surfonic TDA-11®, and Lemon oil.

Another embodiment comprises cyclosporin A, Surfonic TDA-3B®, and Lemon oil.

Another embodiment comprises cyclosporin A, Surfonic TDA-6®, and Lemon oil.

Another embodiment comprises cyclosporin A, Surfonic TDA-6/88®, and Lemon oil.

Another embodiment comprises cyclosporin A, Surfonic TDA-8®, and Lemon oil.

Another embodiment comprises cyclosporin A, Surfonic TDA-8/90®, and Lemon oil.

Another embodiment comprises cyclosporin A, Surfonic TDA-9®, and Lemon oil.

Another embodiment comprises cyclosporin A, Surfonic TX-CCN®, and Lemon oil.

Another embodiment comprises cyclosporin A, Tagat CH 40®, and Lemon oil.

Another embodiment comprises cyclosporin A, Tagat CH 60®, and Lemon oil.

Another embodiment comprises cyclosporin A, Tagat L 2®, and Lemon oil.

Another embodiment comprises cyclosporin A, Tagat S®, and Lemon oil.

Another embodiment comprises cyclosporin A, Tagat S 2®, and Lemon oil.

Another embodiment comprises cyclosporin A, Tally 100 Plus®, and Lemon oil.

Another embodiment comprises cyclosporin A, T-Det A106®, and Lemon oil.

Another embodiment comprises cyclosporin A, T-Det A109®, and Lemon oil.

Another embodiment comprises cyclosporin A, T-Det A136®, and Lemon oil.

Another embodiment comprises cyclosporin A, T-Det A139®, and Lemon oil.

Another embodiment comprises cyclosporin A, T-Det A2412®, and Lemon oil.

Another embodiment comprises cyclosporin A, T-Det A243®, and Lemon oil.

Another embodiment comprises cyclosporin A, T-Det A247®, and Lemon oil.

Another embodiment comprises cyclosporin A, T-Det A249®, and Lemon oil.

Another embodiment comprises cyclosporin A, T-Det A467®, and Lemon oil.

Another embodiment comprises cyclosporin A, T-Det COE®, and Lemon oil.

Another embodiment comprises cyclosporin A, T-Det DD-10®, and Lemon oil.

Another embodiment comprises cyclosporin A, T-Det DD-5®, and Lemon oil.

Another embodiment comprises cyclosporin A, T-Det DD-7®, and Lemon oil.

Another embodiment comprises cyclosporin A, T-Det EPO-104®, and Lemon oil.

Another embodiment comprises cyclosporin A, T-Det EPO-61®, and Lemon oil.

Another embodiment comprises cyclosporin A, T-Det EPO-62®, and Lemon oil.

Another embodiment comprises cyclosporin A, T-Det EPO-64®, and Lemon oil.

Another embodiment comprises cyclosporin A, T-Det N-1.5®, and Lemon oil.

Another embodiment comprises cyclosporin A, T-Det N-10.5®, and Lemon oil.

Another embodiment comprises cyclosporin A, T-Det N-100®, and Lemon oil.

Another embodiment comprises cyclosporin A, T-Det N-1007®, and Lemon oil.

Another embodiment comprises cyclosporin A, T-Det N-12®, and Lemon oil.

Another embodiment comprises cyclosporin A, T-Det N-14®, and Lemon oil.

Another embodiment comprises cyclosporin A, T-Det N-20®, and Lemon oil.

Another embodiment comprises cyclosporin A, T-Det N-30®, and Lemon oil.

Another embodiment comprises cyclosporin A, T-Det N-307®, and Lemon oil.

Another embodiment comprises cyclosporin A, T-Det N-4®, and Lemon oil.

Another embodiment comprises cyclosporin A, T-Det N-40®, and Lemon oil.

Another embodiment comprises cyclosporin A, T-Det N-407®, and Lemon oil.

Another embodiment comprises cyclosporin A, T-Det N-50®, and Lemon oil.

Another embodiment comprises cyclosporin A, T-Det N-507®, and Lemon oil.

Another embodiment comprises cyclosporin A, T-Det N-6®, and Lemon oil.

Another embodiment comprises cyclosporin A, T-Det N-70®, and Lemon oil.

Another embodiment comprises cyclosporin A, T-Det N-8®, and Lemon oil.

Another embodiment comprises cyclosporin A, T-Det N-9.5®, and Lemon oil.

Another embodiment comprises cyclosporin A, T-Det O-12®, and Lemon oil.

Another embodiment comprises cyclosporin A, T-Det O-165®, and Lemon oil.

Another embodiment comprises cyclosporin A, T-Det O-307®, and Lemon oil.

Another embodiment comprises cyclosporin A, T-Det O-40®, and Lemon oil.

Another embodiment comprises cyclosporin A, T-Det O-407®, and Lemon oil.

Another embodiment comprises cyclosporin A, T-Det O-6®, and Lemon oil.

Another embodiment comprises cyclosporin A, T-Det O-8®, and Lemon oil.

Another embodiment comprises cyclosporin A, T-Det O-9®, and Lemon oil.

Another embodiment comprises cyclosporin A, Tegin®, and Lemon oil.

Another embodiment comprises cyclosporin A, Tergitol 15-S-12®, and Lemon oil.

Another embodiment comprises cyclosporin A, Tergitol 15-S-15®, and Lemon oil.

Another embodiment comprises cyclosporin A, Tergitol 15-S-20®, and Lemon oil.

Another embodiment comprises cyclosporin A, Tergitol 15-S-20 (80AQ)®, and Lemon oil.

Another embodiment comprises cyclosporin A, Tergitol 15-S-3®, and Lemon oil.

Another embodiment comprises cyclosporin A, Tergitol 15-S-30®, and Lemon oil.

Another embodiment comprises cyclosporin A, Tergitol 15-S-40®, and Lemon oil.

Another embodiment comprises cyclosporin A, Tergitol 15-S-40 (70AQ)®, and Lemon oil.

Another embodiment comprises cyclosporin A, Tergitol 15-S-5®, and Lemon oil.

Another embodiment comprises cyclosporin A, Tergitol 15-S-7®, and Lemon oil.

Another embodiment comprises cyclosporin A, Tergitol 15-S-9®, and Lemon oil.

Another embodiment comprises cyclosporin A, Tergitol L-101®, and Lemon oil.

Another embodiment comprises cyclosporin A, Tergitol L-61®, and Lemon oil.

Another embodiment comprises cyclosporin A, Tergitol L-62®, and Lemon oil.

Another embodiment comprises cyclosporin A, Tergitol L-64®, and Lemon oil.

Another embodiment comprises cyclosporin A, Tergitol L-81®, and Lemon oil.

Another embodiment comprises cyclosporin A, Tergitol NP-10®, and Lemon oil.

Another embodiment comprises cyclosporin A, Tergitol NP-11®, and Lemon oil.

Another embodiment comprises cyclosporin A, Tergitol NP-12®, and Lemon oil.

Another embodiment comprises cyclosporin A, Tergitol NP-13®, and Lemon oil.

Another embodiment comprises cyclosporin A, Tergitol NP-15®, and Lemon oil.

Another embodiment comprises cyclosporin A, Tergitol NP-30®, and Lemon oil.

Another embodiment comprises cyclosporin A, Tergitol NP-30 (70 AQ)®, and Lemon oil.

Another embodiment comprises cyclosporin A, Tergitol NP-4®, and Lemon oil.

Another embodiment comprises cyclosporin A, Tergitol NP-40®, and Lemon oil.

Another embodiment comprises cyclosporin A, Tergitol NP-40 (70 AQ)®, and Lemon oil.

Another embodiment comprises cyclosporin A, Tergitol NP-50 (70 AQ)®, and Lemon oil.

Another embodiment comprises cyclosporin A, Tergitol NP-55 (70 AQ)®, and Lemon oil.

Another embodiment comprises cyclosporin A, Tergitol NP-6®, and Lemon oil.

Another embodiment comprises cyclosporin A, Tergitol NP-7®, and Lemon oil.

Another embodiment comprises cyclosporin A, Tergitol NP-70 (70 AO)®, and Lemon oil.

Another embodiment comprises cyclosporin A, Tergitol NP-8®, and Lemon oil.

Another embodiment comprises cyclosporin A, Tergitol NP-9®, and Lemon oil.

Another embodiment comprises cyclosporin A, Tergitol NP-9.5®, and Lemon oil.

Another embodiment comprises cyclosporin A, Tergitol TMN-10®, and Lemon oil.

Another embodiment comprises cyclosporin A, Tergitol TMN-3®, and Lemon oil.

Another embodiment comprises cyclosporin A, Tergitol TMN-6®, and Lemon oil.

Another embodiment comprises cyclosporin A, Tergitol XD®, and Lemon oil.

Another embodiment comprises cyclosporin A, Tergitol XH®, and Lemon oil.

Another embodiment comprises cyclosporin A, Tergitol XJ®, and Lemon oil.

Another embodiment comprises cyclosporin A, Tetronic 1107®, and Lemon oil.

Another embodiment comprises cyclosporin A, Tetronic 1307®, and Lemon oil.

Another embodiment comprises cyclosporin A, Tetronic 150R1®, and Lemon oil.

Another embodiment comprises cyclosporin A, Tetronic 304®, and Lemon oil.

Another embodiment comprises cyclosporin A, Tetronic 701®, and Lemon oil.

Another embodiment comprises cyclosporin A, Tetronic 901®, and Lemon oil.

Another embodiment comprises cyclosporin A, Tetronic 904®, and Lemon oil.

Another embodiment comprises cyclosporin A, Tetronic 908®, and Lemon oil.

Another embodiment comprises cyclosporin A, Tetronic 90R4®, and Lemon oil.

Another embodiment comprises cyclosporin A, Tomadol 1-3®, and Lemon oil.

Another embodiment comprises cyclosporin A, Tomadol 1-5®, and Lemon oil.

Another embodiment comprises cyclosporin A, Tomadol 1-7®, and Lemon oil.

Another embodiment comprises cyclosporin A, Tomadol 23-1®, and Lemon oil.

Another embodiment comprises cyclosporin A, Tomadol 23-3®, and Lemon oil.

Another embodiment comprises cyclosporin A, Tomadol 23-5®, and Lemon oil.

Another embodiment comprises cyclosporin A, Tomadol 23-6.5®, and Lemon oil.

Another embodiment comprises cyclosporin A, Tomadol 25-12®, and Lemon oil.

Another embodiment comprises cyclosporin A, Tomadol 25-3®, and Lemon oil.

Another embodiment comprises cyclosporin A, Tomadol 25-7®, and Lemon oil.

Another embodiment comprises cyclosporin A, Tomadol 25-9®, and Lemon oil.

Another embodiment comprises cyclosporin A, Tomadol 45-13®, and Lemon oil.

Another embodiment comprises cyclosporin A, Tomadol 45-2.5®, and Lemon oil.

Another embodiment comprises cyclosporin A, Tomadol 45-7®, and Lemon oil.

Another embodiment comprises cyclosporin A, Tomadol 91-2.5®, and Lemon oil.

Another embodiment comprises cyclosporin A, Tomadol 91-6®, and Lemon oil.

Another embodiment comprises cyclosporin A, Tomadol 91-8®, and Lemon oil.

Another embodiment comprises cyclosporin A, Triton X-100®, and Lemon oil.

Another embodiment comprises cyclosporin A, Triton X-102®, and Lemon oil.

Another embodiment comprises cyclosporin A, Triton X-114®, and Lemon oil.

Another embodiment comprises cyclosporin A, Triton X-15®, and Lemon oil.

Another embodiment comprises cyclosporin A, Triton X-165 (70%)®, and Lemon oil.

Another embodiment comprises cyclosporin A, Triton X-207®, and Lemon oil.

Another embodiment comprises cyclosporin A, Triton X-305 (70%)®, and Lemon oil.

Another embodiment comprises cyclosporin A, Triton X-35®, and Lemon oil.

Another embodiment comprises cyclosporin A, Triton X-405 (70%)®, and Lemon oil.

Another embodiment comprises cyclosporin A, Triton X-45®, and Lemon oil.

Another embodiment comprises cyclosporin A, Triton X-705 (70%)®, and Lemon oil.

Another embodiment comprises cyclosporin A, Volpo S-10®, and Lemon oil.

Another embodiment comprises cyclosporin A, Volpo S-2®, and Lemon oil.

Another embodiment comprises cyclosporin A, Volpo S-20®, and Lemon oil.

Another embodiment comprises cyclosporin A, Volpo-10®, and Lemon oil.

Another embodiment comprises cyclosporin A, Volpo-20®, and Lemon oil.

Another embodiment comprises cyclosporin A, Volpo-3®, and Lemon oil.

Another embodiment comprises cyclosporin A, Volpo-5®, and Lemon oil.

Another embodiment comprises cyclosporin A, Abex 2515®, and Orange oil.

Another embodiment comprises cyclosporin A, Abex 2525/40®, and Orange oil.

Another embodiment comprises cyclosporin A, Abex 2535®, and Orange oil.

Another embodiment comprises cyclosporin A, Abex 2545®, and Orange oil.

Another embodiment comprises cyclosporin A, Acconon E®, and Orange oil.

Another embodiment comprises cyclosporin A, Alfonic 1216CO-9®, and Orange oil.

Another embodiment comprises cyclosporin A, Alfonic 1412-3®, and Orange oil.

Another embodiment comprises cyclosporin A, Alfonic TDA-12®, and Orange oil.

Another embodiment comprises cyclosporin A, Alfonic TDA-3®, and Orange oil.

Another embodiment comprises cyclosporin A, Alfonic TDA-4®, and Orange oil.

Another embodiment comprises cyclosporin A, Alfonic TDA-6®, and Orange oil.

Another embodiment comprises cyclosporin A, Alfonic TDA-7®, and Orange oil.

Another embodiment comprises cyclosporin A, Alfonic TDA-8®, and Orange oil.

Another embodiment comprises cyclosporin A, Alfonic TDA-9®, and Orange oil.

Another embodiment comprises cyclosporin A, Alkamuls 400-DO®, and Orange oil.

Another embodiment comprises cyclosporin A, Alkamuls 400-MO/E®, and Orange oil.

Another embodiment comprises cyclosporin A, Ameroxol OE-10®, and Orange oil.

Another embodiment comprises cyclosporin A, Ameroxol OE-2®, and Orange oil.

Another embodiment comprises cyclosporin A, Ameroxol OE-20®, and Orange oil.

Another embodiment comprises cyclosporin A, Armix 176®, and Orange oil.

Another embodiment comprises cyclosporin A, Armix 180-C®, and Orange oil.

Another embodiment comprises cyclosporin A, Armix 183®, and Orange oil.

Another embodiment comprises cyclosporin A, Brij 35®, and Orange oil.

Another embodiment comprises cyclosporin A, Brij 35 Liquid®, and Orange oil.

Another embodiment comprises cyclosporin A, Brij 35 SP®, and Orange oil.

Another embodiment comprises cyclosporin A, Canasol MJ 2109®, and Orange oil.

Another embodiment comprises cyclosporin A, Canasol MJ 52®, and Orange oil.

Another embodiment comprises cyclosporin A, Canasol R 3600®, and Orange oil.

Another embodiment comprises cyclosporin A, Canasol R 3603®, and Orange oil.

Another embodiment comprises cyclosporin A, Canasol R 4000 H®, and Orange oil.

Another embodiment comprises cyclosporin A, Cedepal CA-210®, and Orange oil.

Another embodiment comprises cyclosporin A, Cedepal CA-520®, and Orange oil.

Another embodiment comprises cyclosporin A, Cedepal CA-630®, and Orange oil.

Another embodiment comprises cyclosporin A, Cedepal CA-720®, and Orange oil.

Another embodiment comprises cyclosporin A, Cedepal CA-890®, and Orange oil.

Another embodiment comprises cyclosporin A, Cedepal CA-897®, and Orange oil.

Another embodiment comprises cyclosporin A, Cedepal CO-210®, and Orange oil.

Another embodiment comprises cyclosporin A, Cedepal CO-430®, and Orange oil.

Another embodiment comprises cyclosporin A, Cedepal CO-530®, and Orange oil.

Another embodiment comprises cyclosporin A, Cedepal CO-630®, and Orange oil.

Another embodiment comprises cyclosporin A, Cedepal CO-710®, and Orange oil.

Another embodiment comprises cyclosporin A, Cedepal CO-730®, and Orange oil.

Another embodiment comprises cyclosporin A, Cedepal CO-880®, and Orange oil.

Another embodiment comprises cyclosporin A, Cedepal CO-887®, and Orange oil.

Another embodiment comprises cyclosporin A, Cedepal CO-890®, and Orange oil.

Another embodiment comprises cyclosporin A, Cedepal CO-897®, and Orange oil.

Another embodiment comprises cyclosporin A, Cedepal CO-970®, and Orange oil.

Another embodiment comprises cyclosporin A, Cedepal CO-977®, and Orange oil.

Another embodiment comprises cyclosporin A, Cedepal CO-990®, and Orange oil.

Another embodiment comprises cyclosporin A, Cedepal CO-997®, and Orange oil.

Another embodiment comprises cyclosporin A, Chemal BP 261®, and Orange oil.

Another embodiment comprises cyclosporin A, Chemal BP 262®, and Orange oil.

Another embodiment comprises cyclosporin A, Chemal BP 3172®, and Orange oil.

Another embodiment comprises cyclosporin A, Chemal BP 3174®, and Orange oil.

Another embodiment comprises cyclosporin A, Chemal DA-4®, and Orange oil.

Another embodiment comprises cyclosporin A, Chemal DA-6®, and Orange oil.

Another embodiment comprises cyclosporin A, Chemal DA-9®, and Orange oil.

Another embodiment comprises cyclosporin A, Chemal LA-23®, and Orange oil.

Another embodiment comprises cyclosporin A, Chemal LA-4®, and Orange oil.

Another embodiment comprises cyclosporin A, Chemal LA-9®, and Orange oil.

Another embodiment comprises cyclosporin A, Chemal OA-20G®, and Orange oil.

Another embodiment comprises cyclosporin A, Chemal OA-5®, and Orange oil.

Another embodiment comprises cyclosporin A, Chemal OA-9®, and Orange oil.

Another embodiment comprises cyclosporin A, Chemal TDA-12®, and Orange oil.

Another embodiment comprises cyclosporin A, Chemal TDA-15®, and Orange oil.

Another embodiment comprises cyclosporin A, Chemal TDA-3®, and Orange oil.

Another embodiment comprises cyclosporin A, Chemal TDA-6®, and Orange oil.

Another embodiment comprises cyclosporin A, Chemal TDA-9®, and Orange oil.

Another embodiment comprises cyclosporin A, Chemax CO-16®, and Orange oil.

Another embodiment comprises cyclosporin A, Chemax CO-200/50®, and Orange oil.

Another embodiment comprises cyclosporin A, Chemax CO-25®, and Orange oil.

Another embodiment comprises cyclosporin A, Chemax CO-30®, and Orange oil.

Another embodiment comprises cyclosporin A, Chemax CO-36®, and Orange oil.

Another embodiment comprises cyclosporin A, Chemax CO-40®, and Orange oil.

Another embodiment comprises cyclosporin A, Chemax CO-5®, and Orange oil.

Another embodiment comprises cyclosporin A, Chemax CO-80®, and Orange oil.

Another embodiment comprises cyclosporin A, Chemax DNP-150/50®, and Orange oil.

Another embodiment comprises cyclosporin A, Chemax DNP-18®, and Orange oil.

Another embodiment comprises cyclosporin A, Chemax DNP-8®, and Orange oil.

Another embodiment comprises cyclosporin A, Chemax E 200 ML®, and Orange oil.

Another embodiment comprises cyclosporin A, Chemax E 200 MO®, and Orange oil.

Another embodiment comprises cyclosporin A, Chemax E 400 ML®, and Orange oil.

Another embodiment comprises cyclosporin A, Chemax E 400 MO®, and Orange oil.

Another embodiment comprises cyclosporin A, Chemax E 600 ML®, and Orange oil.

Another embodiment comprises cyclosporin A, Chemax E 600 MO®, and Orange oil.

Another embodiment comprises cyclosporin A, Chemax E 1000 MO®, and Orange oil.

Another embodiment comprises cyclosporin A, Chemax E 1000 MS®, and Orange oil.

Another embodiment comprises cyclosporin A, Chemax E200 MS®, and Orange oil.

Another embodiment comprises cyclosporin A, Chemax E400 MS®, and Orange oil.

Another embodiment comprises cyclosporin A, Chemax E600 MS®, and Orange oil.

Another embodiment comprises cyclosporin A, Chemax HCO-16®, and Orange oil.

Another embodiment comprises cyclosporin A, Chemax HCO-200/50®, and Orange oil.

Another embodiment comprises cyclosporin A, Chemax HCO-25®, and Orange oil.

Another embodiment comprises cyclosporin A, Chemax HCO-5®, and Orange oil.

Another embodiment comprises cyclosporin A, Chemax TO-16®, and Orange oil.

Another embodiment comprises cyclosporin A, Chemax TO-8®, and Orange oil.

Another embodiment comprises cyclosporin A, Colalube 3409®, and Orange oil.

Another embodiment comprises cyclosporin A, Colalube 3411®, and Orange oil.

Another embodiment comprises cyclosporin A, Colalube 3413®, and Orange oil.

Another embodiment comprises cyclosporin A, Colalube 3414®, and Orange oil.

Another embodiment comprises cyclosporin A, Colalube 3415®, and Orange oil.

Another embodiment comprises cyclosporin A, Colalube 3416®, and Orange oil.

Another embodiment comprises cyclosporin A, Colalube 3417®, and Orange oil.

Another embodiment comprises cyclosporin A, Delonic NPE-100®, and Orange oil.

Another embodiment comprises cyclosporin A, Delonic NPE-30®, and Orange oil.

Another embodiment comprises cyclosporin A, Delonic NPE-40®, and Orange oil.

Another embodiment comprises cyclosporin A, Delonic OPE-10®, and Orange oil.

Another embodiment comprises cyclosporin A, Delonic OPE-12®, and Orange oil.

Another embodiment comprises cyclosporin A, Delonic OPE-30®, and Orange oil.

Another embodiment comprises cyclosporin A, Delonic OPE-40®, and Orange oil.

Another embodiment comprises cyclosporin A, Delonic OPE-5®, and Orange oil.

Another embodiment comprises cyclosporin A, Delonic OPE-7.5®, and Orange oil.

Another embodiment comprises cyclosporin A, DePeg 16-CO®, and Orange oil.

Another embodiment comprises cyclosporin A, DePeg 25-CO®, and Orange oil.

Another embodiment comprises cyclosporin A, DePeg 30-CO®, and Orange oil.

Another embodiment comprises cyclosporin A, DePeg 40-CO®, and Orange oil.

Another embodiment comprises cyclosporin A, DePeg 5-CO®, and Orange oil.

Another embodiment comprises cyclosporin A, DePeg 80-CO®, and Orange oil.

Another embodiment comprises cyclosporin A, DeSonic S-100®, and Orange oil.

Another embodiment comprises cyclosporin A, DeSonic S-114®, and Orange oil.

Another embodiment comprises cyclosporin A, DeSonic S-405®, and Orange oil.

Another embodiment comprises cyclosporin A, DeSonic S-45®, and Orange oil.

Another embodiment comprises cyclosporin A, DeSonic TDA-9®, and Orange oil.

Another embodiment comprises cyclosporin A, DeThox Acid O-14®, and Orange oil.

Another embodiment comprises cyclosporin A, DeThox Acid O-16®, and Orange oil.

Another embodiment comprises cyclosporin A, DeThox Acid O-9®, and Orange oil.

Another embodiment comprises cyclosporin A, DeThox Acid TO-14®, and Orange oil.

Another embodiment comprises cyclosporin A, DeThox Acid TO-16.5®, and Orange oil.

Another embodiment comprises cyclosporin A, DeThox Acid TO-8.5®, and Orange oil.

Another embodiment comprises cyclosporin A, Dur-Em 114®, and Orange oil.

Another embodiment comprises cyclosporin A, Dur-Em 117®, and Orange oil.

Another embodiment comprises cyclosporin A, Dur-Em 204®, and Orange oil.

Another embodiment comprises cyclosporin A, Dur-Em 207®, and Orange oil.

Another embodiment comprises cyclosporin A, Dur-Em 207E®, and Orange oil.

Another embodiment comprises cyclosporin A, Emerest 2704®, and Orange oil.

Another embodiment comprises cyclosporin A, Emerest 2712®, and Orange oil.

Another embodiment comprises cyclosporin A, Emerest 2715®, and Orange oil.

Another embodiment comprises cyclosporin A, Ethal EH-2®, and Orange oil.

Another embodiment comprises cyclosporin A, Ethal EH-5®, and Orange oil.

Another embodiment comprises cyclosporin A, Ethal OA-23®, and Orange oil.

Another embodiment comprises cyclosporin A, Ethosperse CA-20®, and Orange oil.

Another embodiment comprises cyclosporin A, Ethosperse LA-12®, and Orange oil.

Another embodiment comprises cyclosporin A, Ethosperse LA-23®, and Orange oil.

Another embodiment comprises cyclosporin A, Ethosperse LA-4®, and Orange oil.

Another embodiment comprises cyclosporin A, Ethox 3095®, and Orange oil.

Another embodiment comprises cyclosporin A, Ethox CO-16®, and Orange oil.

Another embodiment comprises cyclosporin A, Ethox CO-200®, and Orange oil.

Another embodiment comprises cyclosporin A, Ethox CO-25®, and Orange oil.

Another embodiment comprises cyclosporin A, Ethox CO-30®, and Orange oil.

Another embodiment comprises cyclosporin A, Ethox CO-36®, and Orange oil.

Another embodiment comprises cyclosporin A, Ethox CO-40®, and Orange oil.

Another embodiment comprises cyclosporin A, Ethox CO-5®, and Orange oil.

Another embodiment comprises cyclosporin A, Ethox DO-14®, and Orange oil.

Another embodiment comprises cyclosporin A, Ethox DO-9®, and Orange oil.

Another embodiment comprises cyclosporin A, Ethox MA-8®, and Orange oil.

Another embodiment comprises cyclosporin A, Ethox MI-14®, and Orange oil.

Another embodiment comprises cyclosporin A, Ethox ML-14®, and Orange oil.

Another embodiment comprises cyclosporin A, Ethox ML-5®, and Orange oil.

Another embodiment comprises cyclosporin A, Ethox MO-14®, and Orange oil.

Another embodiment comprises cyclosporin A, Ethox MO-9®, and Orange oil.

Another embodiment comprises cyclosporin A, Ethox MS-14®, and Orange oil.

Another embodiment comprises cyclosporin A, Ethox MS-23®, and Orange oil.

Another embodiment comprises cyclosporin A, Ethox MS-40®, and Orange oil.

Another embodiment comprises cyclosporin A, Ethox MS-8®, and Orange oil.

Another embodiment comprises cyclosporin A, Ethox TO-16®, and Orange oil.

Another embodiment comprises cyclosporin A, Ethox TO-8®, and Orange oil.

Another embodiment comprises cyclosporin A, Ethylan SN-120®, and Orange oil.

Another embodiment comprises cyclosporin A, Ethylan SN-70®, and Orange oil.

Another embodiment comprises cyclosporin A, Ethylan SN-90®, and Orange oil.

Another embodiment comprises cyclosporin A, Flexricin 13®, and Orange oil.

Another embodiment comprises cyclosporin A, Flexricin 15®, and Orange oil.

Another embodiment comprises cyclosporin A, Genapol 1261®, and Orange oil.

Another embodiment comprises cyclosporin A, Generol 122 E25®, and Orange oil.

Another embodiment comprises cyclosporin A, Glicepol 160®, and Orange oil.

Another embodiment comprises cyclosporin A, Glicepol 560®, and Orange oil.

Another embodiment comprises cyclosporin A, Glicepol GMS 20®, and Orange oil.

Another embodiment comprises cyclosporin A, Hetoxide BN-13®, and Orange oil.

Another embodiment comprises cyclosporin A, Hetoxide DNP-4®, and Orange oil.

Another embodiment comprises cyclosporin A, Hetoxide DNP-9.6®, and Orange oil.

Another embodiment comprises cyclosporin A, Hetoxide G-26®, and Orange oil.

Another embodiment comprises cyclosporin A, Hetoxide G-7®, and Orange oil.

Another embodiment comprises cyclosporin A, Hetoxide GT-20®, and Orange oil.

Another embodiment comprises cyclosporin A, Hetoxide HC-40®, and Orange oil.

Another embodiment comprises cyclosporin A, Hetoxol C-24®, and Orange oil.

Another embodiment comprises cyclosporin A, Hetoxol CA-10®, and Orange oil.

Another embodiment comprises cyclosporin A, Hetoxol CA-2®, and Orange oil.

Another embodiment comprises cyclosporin A, Hetoxol CA-20®, and Orange oil.

Another embodiment comprises cyclosporin A, Hetoxol CAWS®, and Orange oil.

Another embodiment comprises cyclosporin A, Hetoxol CD-3®, and Orange oil.

Another embodiment comprises cyclosporin A, Hetoxol CD-4®, and Orange oil.

Another embodiment comprises cyclosporin A, Hetoxol CS-15®, and Orange oil.

Another embodiment comprises cyclosporin A, Hetoxol CS-20®, and Orange oil.

Another embodiment comprises cyclosporin A, Hetoxol CS-20D®, and Orange oil.

Another embodiment comprises cyclosporin A, Hetoxol CS-25®, and Orange oil.

Another embodiment comprises cyclosporin A, Hetoxol CS-50®, and Orange oil.

Another embodiment comprises cyclosporin A, Hetoxol CS-9®, and Orange oil.

Another embodiment comprises cyclosporin A, Hetoxol CSA-15®, and Orange oil.

Another embodiment comprises cyclosporin A, Hetoxol D®, and Orange oil.

Another embodiment comprises cyclosporin A, Hetoxol G®, and Orange oil.

Another embodiment comprises cyclosporin A, Hetoxol J®, and Orange oil.

Another embodiment comprises cyclosporin A, Hetoxol L®, and Orange oil.

Another embodiment comprises cyclosporin A, Hetoxol L-23®, and Orange oil.

Another embodiment comprises cyclosporin A, Hetoxol L-4®, and Orange oil.

Another embodiment comprises cyclosporin A, Hetoxol L-9®, and Orange oil.

Another embodiment comprises cyclosporin A, Hetoxol LS-9®, and Orange oil.

Another embodiment comprises cyclosporin A, Hetoxol M-3®, and Orange oil.

Another embodiment comprises cyclosporin A, Hetoxol OA-10 Special®, and Orange oil.

Another embodiment comprises cyclosporin A, Hetoxol OA-20 Special®, and Orange oil.

Another embodiment comprises cyclosporin A, Hetoxol OA-3 Special®, and Orange oil.

Another embodiment comprises cyclosporin A, Hetoxol OA-5 Special®, and Orange oil.

Another embodiment comprises cyclosporin A, Hetoxol OL-10®, and Orange oil.

Another embodiment comprises cyclosporin A, Hetoxol OL-2®, and Orange oil.

Another embodiment comprises cyclosporin A, Hetoxol OL-23®, and Orange oil.

Another embodiment comprises cyclosporin A, Hetoxol OL-4®, and Orange oil.

Another embodiment comprises cyclosporin A, Hetoxol OL-40®, and Orange oil.

Another embodiment comprises cyclosporin A, Hetoxol OL-5®, and Orange oil.

Another embodiment comprises cyclosporin A, Hetoxol PLA®, and Orange oil.

Another embodiment comprises cyclosporin A, Hetoxol SP-15®, and Orange oil.

Another embodiment comprises cyclosporin A, Hetoxol STA-10®, and Orange oil.

Another embodiment comprises cyclosporin A, Hetoxol STA-2®, and Orange oil.

Another embodiment comprises cyclosporin A, Hetoxol STA-30®, and Orange oil.

Another embodiment comprises cyclosporin A, Hetoxol TD-12®, and Orange oil.

Another embodiment comprises cyclosporin A, Hetoxol TD-18®, and Orange oil.

Another embodiment comprises cyclosporin A, Hetoxol TD-3®, and Orange oil.

Another embodiment comprises cyclosporin A, Hetoxol TD-6®, and Orange oil.

Another embodiment comprises cyclosporin A, Hetoxol TD-9®, and Orange oil.

Another embodiment comprises cyclosporin A, Hetoxol TDEP-15®, and Orange oil.

Another embodiment comprises cyclosporin A, Hetoxol TDEP-63®, and Orange oil.

Another embodiment comprises cyclosporin A, Iconol 24-12®, and Orange oil.

Another embodiment comprises cyclosporin A, Iconol 24-23®, and Orange oil.

Another embodiment comprises cyclosporin A, Iconol 24-4®, and Orange oil.

Another embodiment comprises cyclosporin A, Iconol DA-4®, and Orange oil.

Another embodiment comprises cyclosporin A, Iconol DA-6®, and Orange oil.

Another embodiment comprises cyclosporin A, Iconol TDA-10®, and Orange oil.

Another embodiment comprises cyclosporin A, Iconol TDA-3®, and Orange oil.

Another embodiment comprises cyclosporin A, Iconol TDA-6®, and Orange oil.

Another embodiment comprises cyclosporin A, Iconol TDA-8-90%®, and Orange oil.

Another embodiment comprises cyclosporin A, Iconol TDA-9®, and Orange oil.

Another embodiment comprises cyclosporin A, Igepal CA-210®, and Orange oil.

Another embodiment comprises cyclosporin A, Igepal CA-407®, and Orange oil.

Another embodiment comprises cyclosporin A, Igepal CA-420®, and Orange oil.

Another embodiment comprises cyclosporin A, Igepal CA-520®, and Orange oil.

Another embodiment comprises cyclosporin A, Igepal CA-620®, and Orange oil.

Another embodiment comprises cyclosporin A, Igepal CA-630®, and Orange oil.

Another embodiment comprises cyclosporin A, Igepal CA-720®, and Orange oil.

Another embodiment comprises cyclosporin A, Igepal CA-877®, and Orange oil.

Another embodiment comprises cyclosporin A, Igepal CA-887®, and Orange oil.

Another embodiment comprises cyclosporin A, Igepal CA-897®, and Orange oil.

Another embodiment comprises cyclosporin A, Igepal CO-210®, and Orange oil.

Another embodiment comprises cyclosporin A, Igepal CO-430®, and Orange oil.

Another embodiment comprises cyclosporin A, Igepal CO-520®, and Orange oil.

Another embodiment comprises cyclosporin A, Igepal CO-530®, and Orange oil.

Another embodiment comprises cyclosporin A, Igepal CO-610®, and Orange oil.

Another embodiment comprises cyclosporin A, Igepal CO-630®, and Orange oil.

Another embodiment comprises cyclosporin A, Igepal CO-630 Special®, and Orange oil.

Another embodiment comprises cyclosporin A, Igepal CO-660®, and Orange oil.

Another embodiment comprises cyclosporin A, Igepal CO-710®, and Orange oil.

Another embodiment comprises cyclosporin A, Igepal CO-720®, and Orange oil.

Another embodiment comprises cyclosporin A, Igepal CO-730®, and Orange oil.

Another embodiment comprises cyclosporin A, Igepal CO-738®, and Orange oil.

Another embodiment comprises cyclosporin A, Igepal CO-850®, and Orange oil.

Another embodiment comprises cyclosporin A, Igepal CO-880®, and Orange oil.

Another embodiment comprises cyclosporin A, Igepal CO-887®, and Orange oil.

Another embodiment comprises cyclosporin A, Igepal CO-890®, and Orange oil.

Another embodiment comprises cyclosporin A, Igepal CO-897®, and Orange oil.

Another embodiment comprises cyclosporin A, Igepal CO-970®, and Orange oil.

Another embodiment comprises cyclosporin A, Igepal CO-977®, and Orange oil.

Another embodiment comprises cyclosporin A, Igepal CO-987®, and Orange oil.

Another embodiment comprises cyclosporin A, Igepal CO-990 FLK®, and Orange oil.

Another embodiment comprises cyclosporin A, Igepal CO-997®, and Orange oil.

Another embodiment comprises cyclosporin A, Igepal DM-430®, and Orange oil.

Another embodiment comprises cyclosporin A, Igepal DM-530®, and Orange oil.

Another embodiment comprises cyclosporin A, Igepal DM-710®, and Orange oil.

Another embodiment comprises cyclosporin A, Igepal DM-730®, and Orange oil.

Another embodiment comprises cyclosporin A, Igepal DM-880®, and Orange oil.

Another embodiment comprises cyclosporin A, Igepal DM-970 FLK®, and Orange oil.

Another embodiment comprises cyclosporin A, Igepal OD-410®, and Orange oil.

Another embodiment comprises cyclosporin A, Igepal RC-520®, and Orange oil.

Another embodiment comprises cyclosporin A, Igepal RC-620®, and Orange oil.

Another embodiment comprises cyclosporin A, Igepal RC-630®, and Orange oil.

Another embodiment comprises cyclosporin A, Lexemul CS-20®, and Orange oil.

Another embodiment comprises cyclosporin A, Lipo GMS 450®, and Orange oil.

Another embodiment comprises cyclosporin A, Lipo Polyglycol 1000®, and Orange oil.

Another embodiment comprises cyclosporin A, Lipo Polyglycol 200®, and Orange oil.

Another embodiment comprises cyclosporin A, Lipo Polyglycol 300®, and Orange oil.

Another embodiment comprises cyclosporin A, Lipo Polyglycol 3350®, and Orange oil.

Another embodiment comprises cyclosporin A, Lipo Polyglycol 400®, and Orange oil.

Another embodiment comprises cyclosporin A, Lipo Polyglycol 600®, and Orange oil.

Another embodiment comprises cyclosporin A, Lipocol C-10®, and Orange oil.

Another embodiment comprises cyclosporin A, Lipocol C-2®, and Orange oil.

Another embodiment comprises cyclosporin A, Lipocol C-20®, and Orange oil.

Another embodiment comprises cyclosporin A, Lipocol L-12®, and Orange oil.

Another embodiment comprises cyclosporin A, Lipocol L-23®, and Orange oil.

Another embodiment comprises cyclosporin A, Lipocol L-4®, and Orange oil.

Another embodiment comprises cyclosporin A, Lipocol O-10®, and Orange oil.

Another embodiment comprises cyclosporin A, Lipocol O-2®, and Orange oil.

Another embodiment comprises cyclosporin A, Lipocol O-20®, and Orange oil.

Another embodiment comprises cyclosporin A, Lipocol O-5®, and Orange oil.

Another embodiment comprises cyclosporin A, Lipocol S-10®, and Orange oil.

Another embodiment comprises cyclosporin A, Lipocol S-2®, and Orange oil.

Another embodiment comprises cyclosporin A, Lipocol S-20®, and Orange oil.

Another embodiment comprises cyclosporin A, Lipocol SC-15®, and Orange oil.

Another embodiment comprises cyclosporin A, Lipocol SC-20®, and Orange oil.

Another embodiment comprises cyclosporin A, Lipocol SC-4®, and Orange oil.

Another embodiment comprises cyclosporin A, Lipocol TD-12®, and Orange oil.

Another embodiment comprises cyclosporin A, Lumulse 40-O K®, and Orange oil.

Another embodiment comprises cyclosporin A, Lumulse 42-O K®, and Orange oil.

Another embodiment comprises cyclosporin A, Lumulse GMT-40®, and Orange oil.

Another embodiment comprises cyclosporin A, Lumulse PEG 1450®, and Orange oil.

Another embodiment comprises cyclosporin A, Lumulse PEG 1450 NF®, and Orange oil.

Another embodiment comprises cyclosporin A, Lumulse PEG 8000®, and Orange oil.

Another embodiment comprises cyclosporin A, Lumulse PGO®, and Orange oil.

Another embodiment comprises cyclosporin A, Lumulse POE (2) Oleyl Amine®, and Orange oil.

Another embodiment comprises cyclosporin A, Magrabar PGO-104®, and Orange oil.

Another embodiment comprises cyclosporin A, Mapeg 200 ML®, and Orange oil.

Another embodiment comprises cyclosporin A, Mapeg 400 MO®, and Orange oil.

Another embodiment comprises cyclosporin A, Mapeg 400 MOT®, and Orange oil.

Another embodiment comprises cyclosporin A, Mapeg 400DO®, and Orange oil.

Another embodiment comprises cyclosporin A, Mapeg 400DOT®, and Orange oil.

Another embodiment comprises cyclosporin A, Mapeg 600DOT®, and Orange oil.

Another embodiment comprises cyclosporin A, Marlox B 24/80®, and Orange oil.

Another embodiment comprises cyclosporin A, Marlox FK 86®, and Orange oil.

Another embodiment comprises cyclosporin A, Marlox MO 154®, and Orange oil.

Another embodiment comprises cyclosporin A, Mazox CDA CG®, and Orange oil.

Another embodiment comprises cyclosporin A, Monoglycerides Glyceryl Monestearate®, and Orange oil.

Another embodiment comprises cyclosporin A, Myrj 45®, and Orange oil.

Another embodiment comprises cyclosporin A, Myrj 52®, and Orange oil.

Another embodiment comprises cyclosporin A, Myrj 52S®, and Orange oil.

Another embodiment comprises cyclosporin A, Myrj 53®, and Orange oil.

Another embodiment comprises cyclosporin A, Myrj 59®, and Orange oil.

Another embodiment comprises cyclosporin A, Norfox NP-12®, and Orange oil.

Another embodiment comprises cyclosporin A, Norfox NP-4®, and Orange oil.

Another embodiment comprises cyclosporin A, Norfox NP-6®, and Orange oil.

Another embodiment comprises cyclosporin A, Norfox NP-887®, and Orange oil.

Another embodiment comprises cyclosporin A, Norfox NP-9®, and Orange oil.

Another embodiment comprises cyclosporin A, Norfox NP-977®, and Orange oil.

Another embodiment comprises cyclosporin A, Norfox OP-100®, and Orange oil.

Another embodiment comprises cyclosporin A, Norfox OP-102®, and Orange oil.

Another embodiment comprises cyclosporin A, Norfox OP-114®, and Orange oil.

Another embodiment comprises cyclosporin A, Norfox OP-45®, and Orange oil.

Another embodiment comprises cyclosporin A, Pegosperse 100-L®, and Orange oil.

Another embodiment comprises cyclosporin A, Pegosperse 100-O®, and Orange oil.

Another embodiment comprises cyclosporin A, Pegosperse 100-S®, and Orange oil.

Another embodiment comprises cyclosporin A, Pegosperse 1500-MS®, and-Orange oil.

Another embodiment comprises cyclosporin A, Pegosperse 1750-MS®, and Orange oil.

Another embodiment comprises cyclosporin A, Pegosperse 200 DL®, and Orange oil.

Another embodiment comprises cyclosporin A, Pegosperse 200-ML®, and Orange oil.

Another embodiment comprises cyclosporin A, Pegosperse 400-DL®, and Orange oil.

Another embodiment comprises cyclosporin A, Pegosperse 400-DO®, and Orange oil.

Another embodiment comprises cyclosporin A, Pegosperse 400-DS®, and Orange oil.

Another embodiment comprises cyclosporin A, Pegosperse 400-ML®, and Orange oil.

Another embodiment comprises cyclosporin A, Pegosperse 400-MO®, and Orange oil.

Another embodiment comprises cyclosporin A, Pegosperse 400-MOT®, and Orange oil.

Another embodiment comprises cyclosporin A, Pegosperse 400-MS®, and Orange oil.

Another embodiment comprises cyclosporin A, Pegosperse 50-DS®, and Orange oil.

Another embodiment comprises cyclosporin A, Pegosperse 50-MS®, and Orange oil.

Another embodiment comprises cyclosporin A, Pegosperse 600-ML®, and Orange oil.

Another embodiment comprises cyclosporin A, Pegosperse 600-MS®, and Orange oil.

Another embodiment comprises cyclosporin A, Plurafac A-38®, and Orange oil.

Another embodiment comprises cyclosporin A, Plurafac B-25-5®, and Orange oil.

Another embodiment comprises cyclosporin A, Plurafac B-26®, and Orange oil.

Another embodiment comprises cyclosporin A, Plurafac D-25®, and Orange oil.

Another embodiment comprises cyclosporin A, Plurafac LF 131®, and Orange oil.

Another embodiment comprises cyclosporin A, Plurafac LF 4030®, and Orange oil.

Another embodiment comprises cyclosporin A, Plurafac LF 7000®, and Orange oil.

Another embodiment comprises cyclosporin A, Plurafac RA-20®, and Orange oil.

Another embodiment comprises cyclosporin A, Plurafac RA-30®, and Orange oil.

Another embodiment comprises cyclosporin A, Plurafac RA-40®, and Orange oil.

Another embodiment comprises cyclosporin A, Pluronic 10R5®, and Orange oil.

Another embodiment comprises cyclosporin A, Pluronic 17R2®, and Orange oil.

Another embodiment comprises cyclosporin A, Pluronic 17R4®, and Orange oil.

Another embodiment comprises cyclosporin A, Pluronic 25R2®, and Orange oil.

Another embodiment comprises cyclosporin A, Pluronic 25R4®, and Orange oil.

Another embodiment comprises cyclosporin A, Pluronic 31R1®, and Orange oil.

Another embodiment comprises cyclosporin A, Pluronic F108®, and Orange oil.

Another embodiment comprises cyclosporin A, Pluronic F127®, and Orange oil.

Another embodiment comprises cyclosporin A, Pluronic F38®, and Orange oil.

Another embodiment comprises cyclosporin A, Pluronic F68®, and Orange oil.

Another embodiment comprises cyclosporin A, Pluronic F68LF®, and Orange oil.

Another embodiment comprises cyclosporin A, Pluronic F77®, and Orange oil.

Another embodiment comprises cyclosporin A, Pluronic F87®, and Orange oil.

Another embodiment comprises cyclosporin A, Pluronic F88®, and Orange oil.

Another embodiment comprises cyclosporin A, Pluronic F98®, and Orange oil.

Another embodiment comprises cyclosporin A, Pluronic L10®, and Orange oil.

Another embodiment comprises cyclosporin A, Pluronic L101®, and Orange oil.

Another embodiment comprises cyclosporin A, Pluronic L121®, and Orange oil.

Another embodiment comprises cyclosporin A, Pluronic L31®, and Orange oil.

Another embodiment comprises cyclosporin A, Pluronic L35®, and Orange oil.

Another embodiment comprises cyclosporin A, Pluronic L43®, and Orange oil.

Another embodiment comprises cyclosporin A, Pluronic L44®, and Orange oil.

Another embodiment comprises cyclosporin A, Pluronic L61®, and Orange oil.

Another embodiment comprises cyclosporin A, Pluronic L62®, and Orange oil.

Another embodiment comprises cyclosporin A, Pluronic L62D®, and Orange oil.

Another embodiment comprises cyclosporin A, Pluronic L62LF®, and Orange oil.

Another embodiment comprises cyclosporin A, Pluronic L64®, and Orange oil.

Another embodiment comprises cyclosporin A, Pluronic L81®, and Orange oil.

Another embodiment comprises cyclosporin A, Pluronic L92®, and Orange oil.

Another embodiment comprises cyclosporin A, Pluronic P103®, and Orange oil.

Another embodiment comprises cyclosporin A, Pluronic P104®, and Orange oil.

Another embodiment comprises cyclosporin A, Pluronic P105®, and Orange oil.

Another embodiment comprises cyclosporin A, Pluronic P123®, and Orange oil.

Another embodiment comprises cyclosporin A, Pluronic P65®, and Orange oil.

Another embodiment comprises cyclosporin A, Pluronic P84®, and Orange oil.

Another embodiment comprises cyclosporin A, Pluronic P85®, and Orange oil.

Another embodiment comprises cyclosporin A, Polyaldo 10-2-P®, and Orange oil.

Another embodiment comprises cyclosporin A, Polyaldo DGDO®, and Orange oil.

Another embodiment comprises cyclosporin A, Polyaldo HGDS®, and Orange oil.

Another embodiment comprises cyclosporin A, Polyaldo TGMS®, and Orange oil.

Another embodiment comprises cyclosporin A, Protegin V®, and Orange oil.

Another embodiment comprises cyclosporin A, Protegin W®, and Orange oil.

Another embodiment comprises cyclosporin A, Protegin WX®, and Orange oil.

Another embodiment comprises cyclosporin A, Protegin XV®, and Orange oil.

Another embodiment comprises cyclosporin A, Rhodasurf BC-420®, and Orange oil.

Another embodiment comprises cyclosporin A, Rhodasurf BC-610®, and Orange oil.

Another embodiment comprises cyclosporin A, Rhodasurf BC-630®, and Orange oil.

Another embodiment comprises cyclosporin A, Rhodasurf BC-720®, and Orange oil.

Another embodiment comprises cyclosporin A, Rhodasurf BC-840®, and Orange oil.

Another embodiment comprises cyclosporin A, Rhodasurf L 1®, and Orange oil.

Another embodiment comprises cyclosporin A, Rhodasurf L 12®, and Orange oil.

Another embodiment comprises cyclosporin A, Rhodasurf L 2®, and Orange oil.

Another embodiment comprises cyclosporin A, Rhodasurf L 20®, and Orange oil.

Another embodiment comprises cyclosporin A, Rhodasurf L 3®, and Orange oil.

Another embodiment comprises cyclosporin A, Rhodasurf L 30®, and Orange oil.

Another embodiment comprises cyclosporin A, Rhodasurf L 4®, and Orange oil.

Another embodiment comprises cyclosporin A, Rhodasurf L 50®, and Orange oil.

Another embodiment comprises cyclosporin A, Rhodasurf L 7/90®, and Orange oil.

Another embodiment comprises cyclosporin A, Rhodasurf LA-3®, and Orange oil.

Another embodiment comprises cyclosporin A, Rhodasurf LA-7®, and Orange oil.

Another embodiment comprises cyclosporin A, Rhodasurf LAN-23®, and Orange oil.

Another embodiment comprises cyclosporin A, Rhodasurf ON-870®, and Orange oil.

Another embodiment comprises cyclosporin A, Rhodasurf ON-877®, and Orange oil.

Another embodiment comprises cyclosporin A, Rhodasurf TDA-8/5®, and Orange oil.

Another embodiment comprises cyclosporin A, Ritapro 100®, and Orange oil.

Another embodiment comprises cyclosporin A, Ritapro 165®, and Orange oil.

Another embodiment comprises cyclosporin A, Ritapro 200®, and Orange oil.

Another embodiment comprises cyclosporin A, Ritapro 300®, and Orange oil.

Another embodiment comprises cyclosporin A, Ritoleth 10®, and Orange oil.

Another embodiment comprises cyclosporin A, Ritoleth 2®, and Orange oil.

Another embodiment comprises cyclosporin A, Ritoleth 20®, and Orange oil.

Another embodiment comprises cyclosporin A, Ritoleth 5®, and Orange oil.

Another embodiment comprises cyclosporin A, Santone 10-10-0®, and Orange oil.

Another embodiment comprises cyclosporin A, Santone 3-1-S XTR®, and Orange oil.

Another embodiment comprises cyclosporin A, Santone 3-1-SH®, and Orange oil.

Another embodiment comprises cyclosporin A, Santone 8-1-O®, and Orange oil.

Another embodiment comprises cyclosporin A, Solulan 16®, and Orange oil.

Another embodiment comprises cyclosporin A, Solulan 75®, and Orange oil.

Another embodiment comprises cyclosporin A, Solulan C-24®, and Orange oil.

Another embodiment comprises cyclosporin A, Solulan L-575®, and Orange oil.

Another embodiment comprises cyclosporin A, Standamox CAW®, and Orange oil.

Another embodiment comprises cyclosporin A, Surfine AZI-A®, and Orange oil.

Another embodiment comprises cyclosporin A, Surfonic CO-15®, and Orange oil.

Another embodiment comprises cyclosporin A, Surfonic CO-25®, and Orange oil.

Another embodiment comprises cyclosporin A, Surfonic CO-30®, and Orange oil.

Another embodiment comprises cyclosporin A, Surfonic CO-36®, and Orange oil.

Another embodiment comprises cyclosporin A, Surfonic CO-42®, and Orange oil.

Another embodiment comprises cyclosporin A, Surfonic E400-MO®, and Orange oil.

Another embodiment comprises cyclosporin A, Surfonic L12-3®, and Orange oil.

Another embodiment comprises cyclosporin A, Surfonic L12-6®, and Orange oil.

Another embodiment comprises cyclosporin A, Surfonic L12-8®, and Orange oil.

Another embodiment comprises cyclosporin A, Surfonic L24-12®, and Orange oil.

Another embodiment comprises cyclosporin A, Surfonic L24-2®, and Orange oil.

Another embodiment comprises cyclosporin A, Surfonic L24-22®, and Orange oil.

Another embodiment comprises cyclosporin A, Surfonic L24-3®, and Orange oil.

Another embodiment comprises cyclosporin A, Surfonic L24-4®, and Orange oil.

Another embodiment comprises cyclosporin A, Surfonic L24-5®, and Orange oil.

Another embodiment comprises cyclosporin A, Surfonic L24-7®, and Orange oil.

Another embodiment comprises cyclosporin A, Surfonic L24-7.1®, and Orange oil.

Another embodiment comprises cyclosporin A, Surfonic L24-9®, and Orange oil.

Another embodiment comprises cyclosporin A, Surfonic LF-17®, and Orange oil.

Another embodiment comprises cyclosporin A, Surfonic LF-18®, and Orange oil.

Another embodiment comprises cyclosporin A, Surfonic LF-37®, and Orange oil.

Another embodiment comprises cyclosporin A, Surfonic LF-41®, and Orange oil.

Another embodiment comprises cyclosporin A, Surfonic LF-42®, and Orange oil.

Another embodiment comprises cyclosporin A, Surfonic LF-47®, and Orange oil.

Another embodiment comprises cyclosporin A, Surfonic LF-68®, and Orange oil.

Another embodiment comprises cyclosporin A, Surfonic N-10®, and Orange oil.

Another embodiment comprises cyclosporin A, Surfonic N-100®, and Orange oil.

Another embodiment comprises cyclosporin A, Surfonic N-102®, and Orange oil.

Another embodiment comprises cyclosporin A, Surfonic N-120®, and Orange oil.

Another embodiment comprises cyclosporin A, Surfonic N-150®, and Orange oil.

Another embodiment comprises cyclosporin A, Surfonic N-200®, and Orange oil.

Another embodiment comprises cyclosporin A, Surfonic N-300®, and Orange oil.

Another embodiment comprises cyclosporin A, Surfonic N-31.5®, and Orange oil.

Another embodiment comprises cyclosporin A, Surfonic N-40®, and Orange oil.

Another embodiment comprises cyclosporin A, Surfonic N-400®, and Orange oil.

Another embodiment comprises cyclosporin A, Surfonic N-60®, and Orange oil.

Another embodiment comprises cyclosporin A, Surfonic N-85®, and Orange oil.

Another embodiment comprises cyclosporin A, Surfonic N-95®, and Orange oil.

Another embodiment comprises cyclosporin A, Surfonic NB-18®, and Orange oil.

Another embodiment comprises cyclosporin A, Surfonic NB-23®, and Orange oil.

Another embodiment comprises cyclosporin A, Surfonic NB-25®, and Orange oil.

Another embodiment comprises cyclosporin A, Surfonic NB-307®, and Orange oil.

Another embodiment comprises cyclosporin A, Surfonic NB-31®, and Orange oil.

Another embodiment comprises cyclosporin A, Surfonic NB-41®, and Orange oil.

Another embodiment comprises cyclosporin A, Surfonic NB-43®, and Orange oil.

Another embodiment comprises cyclosporin A, Surfonic OP-100®, and Orange oil.

Another embodiment comprises cyclosporin A, Surfonic OP-120®, and Orange oil.

Another embodiment comprises cyclosporin A, Surfonic OP-15®, and Orange oil.

Another embodiment comprises cyclosporin A, Surfonic OP-35®, and Orange oil.

Another embodiment comprises cyclosporin A, Surfonic OP-70®, and Orange oil.

Another embodiment comprises cyclosporin A, Surfonic P1®, and Orange oil.

Another embodiment comprises cyclosporin A, Surfonic P3®, and Orange oil.

Another embodiment comprises cyclosporin A, Surfonic P5®, and Orange oil.

Another embodiment comprises cyclosporin A, Surfonic P6®, and Orange oil.

Another embodiment comprises cyclosporin A, Surfonic POA-17R2®, and Orange oil.

Another embodiment comprises cyclosporin A, Surfonic POA-25R2®, and Orange oil.

Another embodiment comprises cyclosporin A, Surfonic TDA-11®, and Orange oil.

Another embodiment comprises cyclosporin A, Surfonic TDA-3B®, and Orange oil.

Another embodiment comprises cyclosporin A, Surfonic TDA-6®, and Orange oil.

Another embodiment comprises cyclosporin A, Surfonic TDA-6/88®, and Orange oil.

Another embodiment comprises cyclosporin A, Surfonic TDA-8®, and Orange oil.

Another embodiment comprises cyclosporin A, Surfonic TDA-8/90®, and Orange oil.

Another embodiment comprises cyclosporin A, Surfonic TDA-9®, and Orange oil.

Another embodiment comprises cyclosporin A, Surfonic TX-CCN®, and Orange oil.

Another embodiment comprises cyclosporin A, Tagat CH 40®, and Orange oil.

Another embodiment comprises cyclosporin A, Tagat CH 60®, and Orange oil.

Another embodiment comprises cyclosporin A, Tagat L 2®, and Orange oil.

Another embodiment comprises cyclosporin A, Tagat S®, and Orange oil.

Another embodiment comprises cyclosporin A, Tagat S 2®, and Orange oil.

Another embodiment comprises cyclosporin A, Tally 100 Plus®, and Orange oil.

Another embodiment comprises cyclosporin A, T-Det A106®, and Orange oil.

Another embodiment comprises cyclosporin A, T-Det A109®, and Orange oil.

Another embodiment comprises cyclosporin A, T-Det A136®, and Orange oil.

Another embodiment comprises cyclosporin A, T-Det A139®, and Orange oil.

Another embodiment comprises cyclosporin A, T-Det A2412®, and Orange oil.

Another embodiment comprises cyclosporin A, T-Det A243®, and Orange oil.

Another embodiment comprises cyclosporin A, T-Det A247®, and Orange oil.

Another embodiment comprises cyclosporin A, T-Det A249®, and Orange oil.

Another embodiment comprises cyclosporin A, T-Det A467®, and Orange oil.

Another embodiment comprises cyclosporin A, T-Det COE®, and Orange oil.

Another embodiment comprises cyclosporin A, T-Det DD-10®, and Orange oil.

Another embodiment comprises cyclosporin A, T-Det DD-5®, and Orange oil.

Another embodiment comprises cyclosporin A, T-Det DD-7®, and Orange oil.

Another embodiment comprises cyclosporin A, T-Det EPO-104®, and Orange oil.

Another embodiment comprises cyclosporin A, T-Det EPO-61®, and Orange oil.

Another embodiment comprises cyclosporin A, T-Det EPO-62®, and Orange oil.

Another embodiment comprises cyclosporin A, T-Det EPO-64®, and Orange oil.

Another embodiment comprises cyclosporin A, T-Det N-1.5®, and Orange oil.

Another embodiment comprises cyclosporin A, T-Det N-10.5®, and Orange oil.

Another embodiment comprises cyclosporin A, T-Det N-100®, and Orange oil.

Another embodiment comprises cyclosporin A, T-Det N-1007®, and Orange oil.

Another embodiment comprises cyclosporin A, T-Det N-12®, and Orange oil.

Another embodiment comprises cyclosporin A, T-Det N-14®, and Orange oil.

Another embodiment comprises cyclosporin A, T-Det N-20®, and Orange oil.

Another embodiment comprises cyclosporin A, T-Det N-30®, and Orange oil.

Another embodiment comprises cyclosporin A, T-Det N-307®, and Orange oil.

Another embodiment comprises cyclosporin A, T-Det N-4®, and Orange oil.

Another embodiment comprises cyclosporin A, T-Det N-40®, and Orange oil.

Another embodiment comprises cyclosporin A, T-Det N-407®, and Orange oil.

Another embodiment comprises cyclosporin A, T-Det N-50®, and Orange oil.

Another embodiment comprises cyclosporin A, T-Det N-507®, and Orange oil.

Another embodiment comprises cyclosporin A, T-Det N-6®, and Orange oil.

Another embodiment comprises cyclosporin A, T-Det N-70®, and Orange oil.

Another embodiment comprises cyclosporin A, T-Det N-8®, and Orange oil.

Another embodiment comprises cyclosporin A, T-Det N-9.5®, and Orange oil.

Another embodiment comprises cyclosporin A, T-Det O-12®, and Orange oil.

Another embodiment comprises cyclosporin A, T-Det O-165®, and Orange oil.

Another embodiment comprises cyclosporin A, T-Det O-307®, and Orange oil.

Another embodiment comprises cyclosporin A, T-Det O-40®, and Orange oil.

Another embodiment comprises cyclosporin A, T-Det O-407®, and Orange oil.

Another embodiment comprises cyclosporin A, T-Det O-6®, and Orange oil.

Another embodiment comprises cyclosporin A, T-Det O-8®, and Orange oil.

Another embodiment comprises cyclosporin A, T-Det O-9®, and Orange oil.

Another embodiment comprises cyclosporin A, Tegin®, and Orange oil.

Another embodiment comprises cyclosporin A, Tergitol 15-S-12®, and Orange oil.

Another embodiment comprises cyclosporin A, Tergitol 15-S-15®, and Orange oil.

Another embodiment comprises cyclosporin A, Tergitol 15-S-20®, and Orange oil.

Another embodiment comprises cyclosporin A, Tergitol 15-S-20 (80AQ)®, and Orange oil.

Another embodiment comprises cyclosporin A, Tergitol 15-S-3®, and Orange oil.

Another embodiment comprises cyclosporin A, Tergitol 15-S-30®, and Orange oil.

Another embodiment comprises cyclosporin A, Tergitol 15-S-40®, and Orange oil.

Another embodiment comprises cyclosporin A, Tergitol 15-S-40 (70AQ)®, and Orange oil.

Another embodiment comprises cyclosporin A, Tergitol 15-S-5®, and Orange oil.

Another embodiment comprises cyclosporin A, Tergitol 15-S-7®, and Orange oil.

Another embodiment comprises cyclosporin A, Tergitol 15-S-9®, and Orange oil.

Another embodiment comprises cyclosporin A, Tergitol L-101®, and Orange oil.

Another embodiment comprises cyclosporin A, Tergitol L-61®, and Orange oil.

Another embodiment comprises cyclosporin A, Tergitol L-62®, and Orange oil.

Another embodiment comprises cyclosporin A, Tergitol L-64®, and Orange oil.

Another embodiment comprises cyclosporin A, Tergitol L-81®, and Orange oil.

Another embodiment comprises cyclosporin A, Tergitol NP-10®, and Orange oil.

Another embodiment comprises cyclosporin A, Tergitol NP-11®, and Orange oil.

Another embodiment comprises cyclosporin A, Tergitol NP-12®, and Orange oil.

Another embodiment comprises cyclosporin A, Tergitol NP-13®, and Orange oil.

Another embodiment comprises cyclosporin A, Tergitol NP-15®, and Orange oil.

Another embodiment comprises cyclosporin A, Tergitol NP-30®, and Orange oil.

Another embodiment comprises cyclosporin A, Tergitol NP-30 (70 AQ)®, and Orange oil.

Another embodiment comprises cyclosporin A, Tergitol NP-4®, and Orange oil.

Another embodiment comprises cyclosporin A, Tergitol NP-40®, and Orange oil.

Another embodiment comprises cyclosporin A, Tergitol NP-40 (70 AQ)®, and Orange oil.

Another embodiment comprises cyclosporin A, Tergitol NP-50 (70 AQ)®, and Orange oil.

Another embodiment comprises cyclosporin A, Tergitol NP-55 (70 AQ)®, and Orange oil.

Another embodiment comprises cyclosporin A, Tergitol NP-6®, and Orange oil.

Another embodiment comprises cyclosporin A, Tergitol NP-7®, and Orange oil.

Another embodiment comprises cyclosporin A, Tergitol NP-70 (70 AQ)®, and Orange oil.

Another embodiment comprises cyclosporin A, Tergitol NP-8®, and Orange oil.

Another embodiment comprises cyclosporin A, Tergitol NP-9®, and Orange oil.

Another embodiment comprises cyclosporin A, Tergitol NP-9.5®, and Orange oil.

Another embodiment comprises cyclosporin A, Tergitol TMN-10®, and Orange oil.

Another embodiment comprises cyclosporin A, Tergitol TMN-3®, and Orange oil.

Another embodiment comprises cyclosporin A, Tergitol TMN-6®, and Orange oil.

Another embodiment comprises cyclosporin A, Tergitol XD®, and Orange oil.

Another embodiment comprises cyclosporin A, Tergitol XH®, and Orange oil.

Another embodiment comprises cyclosporin A, Tergitol XJ®, and Orange oil.

Another embodiment comprises cyclosporin A, Tetronic 1107®, and Orange oil.

Another embodiment comprises cyclosporin A, Tetronic 1307®, and Orange oil.

Another embodiment comprises cyclosporin A, Tetronic 150R1®, and Orange oil.

Another embodiment comprises cyclosporin A, Tetronic 304®, and Orange oil.

Another embodiment comprises cyclosporin A, Tetronic 701®, and Orange oil.

Another embodiment comprises cyclosporin A, Tetronic 901®, and Orange oil.

Another embodiment comprises cyclosporin A, Tetronic 904®, and Orange oil.

Another embodiment comprises cyclosporin A, Tetronic 908®, and Orange oil.

Another embodiment comprises cyclosporin A, Tetronic 90R4®, and Orange oil.

Another embodiment comprises cyclosporin A, Tomadol 1-3®, and Orange oil.

Another embodiment comprises cyclosporin A, Tomadol 1-5®, and Orange oil.

Another embodiment comprises cyclosporin A, Tomadol 1-7®, and Orange oil.

Another embodiment comprises cyclosporin A, Tomadol 23-1®, and Orange oil.

Another embodiment comprises cyclosporin A, Tomadol 23-3®, and Orange oil.

Another embodiment comprises cyclosporin A, Tomadol 23-5®, and Orange oil.

Another embodiment comprises cyclosporin A, Tomadol 23-6.5®, and Orange oil.

Another embodiment comprises cyclosporin A, Tomadol 25-12®, and Orange oil.

Another embodiment comprises cyclosporin A, Tomadol 25-3®, and Orange oil.

Another embodiment comprises cyclosporin A, Tomadol 25-7®, and Orange oil.

Another embodiment comprises cyclosporin A, Tomadol 25-9®, and Orange oil.

Another embodiment comprises cyclosporin A, Tomadol 45-13®, and Orange oil.

Another embodiment comprises cyclosporin A, Tomadol 45-2.5®, and Orange oil.

Another embodiment comprises cyclosporin A, Tomadol 45-7®, and Orange oil.

Another embodiment comprises cyclosporin A, Tomadol 91-2.5®, and Orange oil.

Another embodiment comprises cyclosporin A, Tomadol 91-6®, and Orange oil.

Another embodiment comprises cyclosporin A, Tomadol 91-8®, and Orange oil.

Another embodiment comprises cyclosporin A, Triton X-100®, and Orange oil.

Another embodiment comprises cyclosporin A, Triton X-102®, and Orange oil.

Another embodiment comprises cyclosporin A, Triton X-114®, and Orange oil.

Another embodiment comprises cyclosporin A, Triton X-15®, and Orange oil.

Another embodiment comprises cyclosporin A, Triton X-165 (70%)®, and Orange oil.

Another embodiment comprises cyclosporin A, Triton X-207®, and Orange oil.

Another embodiment comprises cyclosporin A, Triton X-305 (70%)®, and Orange oil.

Another embodiment comprises cyclosporin A, Triton X-35®, and Orange oil.

Another embodiment comprises cyclosporin A, Triton X-405 (70%)®, and Orange oil.

Another embodiment comprises cyclosporin A, Triton X-45®, and Orange oil.

Another embodiment comprises cyclosporin A, Triton X-705 (70%)®, and Orange oil.

Another embodiment comprises cyclosporin A, Volpo S-10®, and Orange oil.

Another embodiment comprises cyclosporin A, Volpo S-2®, and Orange oil.

Another embodiment comprises cyclosporin A, Volpo S-20®, and Orange oil.

Another embodiment comprises cyclosporin A, Volpo-10®, and Orange oil.

Another embodiment comprises cyclosporin A, Volpo-20®, and Orange oil.

Another embodiment comprises cyclosporin A, Volpo-3®, and Orange oil.

Another embodiment comprises cyclosporin A, Volpo-5®, and Orange oil.

Another embodiment comprises cyclosporin A, Abex 2515®, and Clary sage oil.

Another embodiment comprises cyclosporin A, Abex 2525/40®, and Clary sage oil.

Another embodiment comprises cyclosporin A, Abex 2535®, and Clary sage oil.

Another embodiment comprises cyclosporin A, Abex 2545®, and Clary sage oil.

Another embodiment comprises cyclosporin A, Acconon E®, and Clary sage oil.

Another embodiment comprises cyclosporin A, Alfonic 1216CO-9®, and Clary sage oil.

Another embodiment comprises cyclosporin A, Alfonic 1412-3®, and Clary sage oil.

Another embodiment comprises cyclosporin A, Alfonic TDA-12®, and Clary sage oil.

Another embodiment comprises cyclosporin A, Alfonic TDA-3®, and Clary sage oil.

Another embodiment comprises cyclosporin A, Alfonic TDA-4®, and Clary sage oil.

Another embodiment comprises cyclosporin A, Alfonic TDA-6®, and Clary sage oil.

Another embodiment comprises cyclosporin A, Alfonic TDA-7®, and Clary sage oil.

Another embodiment comprises cyclosporin A, Alfonic TDA-8®, and Clary sage oil.

Another embodiment comprises cyclosporin A, Alfonic TDA-9®, and Clary sage oil.

Another embodiment comprises cyclosporin A, Alkamuls 400-DO®, and Clary sage oil.

Another embodiment comprises cyclosporin A, Alkamuls 400-MO/E®, and Clary sage oil.

Another embodiment comprises cyclosporin A, Ameroxol OE-10®, and Clary sage oil.

Another embodiment comprises cyclosporin A, Ameroxol OE-2®, and Clary sage oil.

Another embodiment comprises cyclosporin A, Ameroxol OE-20®, and Clary sage oil.

Another embodiment comprises cyclosporin A, Armix 176®, and Clary sage oil.

Another embodiment comprises cyclosporin A, Armix 180-C®, and Clary sage oil.

Another embodiment comprises cyclosporin A, Armix 183®, and Clary sage oil.

Another embodiment comprises cyclosporin A, Brij 35®, and Clary sage oil.

Another embodiment comprises cyclosporin A, Brij 35 Liquid®, and Clary sage oil.

Another embodiment comprises cyclosporin A, Brij 35 SP®, and Clary sage oil.

Another embodiment comprises cyclosporin A, Canasol MJ 2109®, and Clary sage oil.

Another embodiment comprises cyclosporin A, Canasol MJ 52®, and Clary sage oil.

Another embodiment comprises cyclosporin A, Canasol R 3600®, and Clary sage oil.

Another embodiment comprises cyclosporin A, Canasol R 3603®, and Clary sage oil.

Another embodiment comprises cyclosporin A, Canasol R 4000 H®, and Clary sage oil.

Another embodiment comprises cyclosporin A, Cedepal CA-210®, and Clary sage oil.

Another embodiment comprises cyclosporin A, Cedepal CA-520®, and Clary sage oil.

Another embodiment comprises cyclosporin A, Cedepal CA-630®, and Clary sage oil.

Another embodiment comprises cyclosporin A, Cedepal CA-720®, and Clary sage oil.

Another embodiment comprises cyclosporin A, Cedepal CA-890®, and Clary sage oil.

Another embodiment comprises cyclosporin A, Cedepal CA-897®, and Clary sage oil.

Another embodiment comprises cyclosporin A, Cedepal CO-210®, and Clary sage oil.

Another embodiment comprises cyclosporin A, Cedepal CO-430®, and Clary sage oil.

Another embodiment comprises cyclosporin A, Cedepal CO-530®, and Clary sage oil.

Another embodiment comprises cyclosporin A, Cedepal CO-630®, and Clary sage oil.

Another embodiment comprises cyclosporin A, Cedepal CO-710®, and Clary sage oil.

Another embodiment comprises cyclosporin A, Cedepal CO-730®, and Clary sage oil.

Another embodiment comprises cyclosporin A, Cedepal CO-880®, and Clary sage oil.

Another embodiment comprises cyclosporin A, Cedepal CO-887®, and Clary sage oil.

Another embodiment comprises cyclosporin A, Cedepal CO-890®, and Clary sage oil.

Another embodiment comprises cyclosporin A, Cedepal CO-897®, and Clary sage oil.

Another embodiment comprises cyclosporin A, Cedepal CO-970®, and Clary sage oil.

Another embodiment comprises cyclosporin A, Cedepal CO-977®, and Clary sage oil.

Another embodiment comprises cyclosporin A, Cedepal CO-990®, and Clary sage oil.

Another embodiment comprises cyclosporin A, Cedepal CO-997®, and Clary sage oil.

Another embodiment comprises cyclosporin A, Chemal BP 261®, and Clary sage oil.

Another embodiment comprises cyclosporin A, Chemal BP 262®, and Clary sage oil.

Another embodiment comprises cyclosporin A, Chemal BP 3172®, and Clary sage oil.

Another embodiment comprises cyclosporin A, Chemal BP 3174®, and Clary sage oil.

Another embodiment comprises cyclosporin A, Chemal DA-4®, and Clary sage oil.

Another embodiment comprises cyclosporin A, Chemal DA-6®, and Clary sage oil.

Another embodiment comprises cyclosporin A, Chemal DA-9®, and Clary sage oil.

Another embodiment comprises cyclosporin A, Chemal LA-23®, and Clary sage oil.

Another embodiment comprises cyclosporin A, Chemal LA-4®, and Clary sage oil.

Another embodiment comprises cyclosporin A, Chemal LA-9®, and Clary sage oil.

Another embodiment comprises cyclosporin A, Chemal OA-20G®, and Clary sage oil.

Another embodiment comprises cyclosporin A, Chemal OA-5®, and Clary sage oil.

Another embodiment comprises cyclosporin A, Chemal OA-9®, and Clary sage oil.

Another embodiment comprises cyclosporin A, Chemal TDA-12®, and Clary sage oil.

Another embodiment comprises cyclosporin A, Chemal TDA-15®, and Clary sage oil.

Another embodiment comprises cyclosporin A, Chemal TDA-3®, and Clary sage oil.

Another embodiment comprises cyclosporin A, Chemal TDA-6®, and Clary sage oil.

Another embodiment comprises cyclosporin A, Chemal TDA-9®, and Clary sage oil.

Another embodiment comprises cyclosporin A, Chemax CO-16®, and Clary sage oil.

Another embodiment comprises cyclosporin A, Chemax CO-200/50®, and Clary sage oil.

Another embodiment comprises cyclosporin A, Chemax CO-25®, and Clary sage oil.

Another embodiment comprises cyclosporin A, Chemax CO-30®, and Clary sage oil.

Another embodiment comprises cyclosporin A, Chemax CO-36®, and Clary sage oil.

Another embodiment comprises cyclosporin A, Chemax CO-40®, and Clary sage oil.

Another embodiment comprises cyclosporin A, Chemax CO-5®, and Clary sage oil.

Another embodiment comprises cyclosporin A, Chemax CO-80®, and Clary sage oil.

Another embodiment comprises cyclosporin A, Chemax DNP-150/50®, and Clary sage oil.

Another embodiment comprises cyclosporin A, Chemax DNP-18®, and Clary sage oil.

Another embodiment comprises cyclosporin A, Chemax DNP-8®, and Clary sage oil.

Another embodiment comprises cyclosporin A, Chemax E 200 ML®, and Clary sage oil.

Another embodiment comprises cyclosporin A, Chemax E 200 MO®, and Clary sage oil.

Another embodiment comprises cyclosporin A, Chemax E 400 ML®, and Clary sage oil.

Another embodiment comprises cyclosporin A, Chemax E 400 MO®, and Clary sage oil.

Another embodiment comprises cyclosporin A, Chemax E 600 ML®, and Clary sage oil.

Another embodiment comprises cyclosporin A, Chemax E 600 MO®, and Clary sage oil.

Another embodiment comprises cyclosporin A, Chemax E 1000 MO®, and Clary sage oil.

Another embodiment comprises cyclosporin A, Chemax E 1000 MS®, and Clary sage oil.

Another embodiment comprises cyclosporin A, Chemax E200 MS®, and Clary sage oil.

Another embodiment comprises cyclosporin A, Chemax E400 MS®, and Clary sage oil.

Another embodiment comprises cyclosporin A, Chemax E600 MS®, and Clary sage oil.

Another embodiment comprises cyclosporin A, Chemax HCO-16®, and Clary sage oil.

Another embodiment comprises cyclosporin A, Chemax HCO-200/50®, and Clary sage oil.

Another embodiment comprises cyclosporin A, Chemax HCO-25®, and Clary sage oil.

Another embodiment comprises cyclosporin A, Chemax HCO-5®, and Clary sage oil.

Another embodiment comprises cyclosporin A, Chemax TO-16®, and Clary sage oil.

Another embodiment comprises cyclosporin A, Chemax TO-8®, and Clary sage oil.

Another embodiment comprises cyclosporin A, Colalube 3409®, and Clary sage oil.

Another embodiment comprises cyclosporin A, Colalube 3411®, and Clary sage oil.

Another embodiment comprises cyclosporin A, Colalube 3413®, and Clary sage oil.

Another embodiment comprises cyclosporin A, Colalube 3414®, and Clary sage oil.

Another embodiment comprises cyclosporin A, Colalube 3415®, and Clary sage oil.

Another embodiment comprises cyclosporin A, Colalube 3416®, and Clary sage oil.

Another embodiment comprises cyclosporin A, Colalube 3417®, and Clary sage oil.

Another embodiment comprises cyclosporin A, Delonic NPE-100®, and Clary sage oil.

Another embodiment comprises cyclosporin A, Delonic NPE-30®, and Clary sage oil.

Another embodiment comprises cyclosporin A, Delonic NPE-40®, and Clary sage oil.

Another embodiment comprises cyclosporin A, Delonic OPE-10®, and Clary sage oil.

Another embodiment comprises cyclosporin A, Delonic OPE-12®, and Clary sage oil.

Another embodiment comprises cyclosporin A, Delonic OPE-30®, and Clary sage oil.

Another embodiment comprises cyclosporin A, Delonic OPE-40®, and Clary sage oil.

Another embodiment comprises cyclosporin A, Delonic OPE-5®, and Clary sage oil.

Another embodiment comprises cyclosporin A, Delonic OPE-7.5®, and Clary sage oil.

Another embodiment comprises cyclosporin A, DePeg 16-CO®, and Clary sage oil.

Another embodiment comprises cyclosporin A, DePeg 25-CO®, and Clary sage oil.

Another embodiment comprises cyclosporin A, DePeg 30-CO, and Clary sage oil.

Another embodiment comprises cyclosporin A, DePeg 40-CO®, and Clary sage oil.

Another embodiment comprises cyclosporin A, DePeg 5-CO®, and Clary sage oil.

Another embodiment comprises cyclosporin A, DePeg 80-CO®, and Clary sage oil.

Another embodiment comprises cyclosporin A, DeSonic S-100®, and Clary sage oil.

Another embodiment comprises cyclosporin A, DeSonic S-114®, and Clary sage oil.

Another embodiment comprises cyclosporin A, DeSonic S-405®, and Clary sage oil.

Another embodiment comprises cyclosporin A, DeSonic S-45®, and Clary sage oil.

Another embodiment comprises cyclosporin A, DeSonic TDA-9®, and Clary sage oil.

Another embodiment comprises cyclosporin A, DeThox Acid O-14®, and Clary sage oil.

Another embodiment comprises cyclosporin A, DeThox Acid O-16®, and Clary sage oil.

Another embodiment comprises cyclosporin A, DeThox Acid O-9®, and Clary sage oil.

Another embodiment comprises cyclosporin A, DeThox Acid TO-14®, and Clary sage oil.

Another embodiment comprises cyclosporin A, DeThox Acid TO-16.5®, and Clary sage oil.

Another embodiment comprises cyclosporin A, DeThox Acid TO-8.5®, and Clary sage oil.

Another embodiment comprises cyclosporin A, Dur-Em 114®, and Clary sage oil.

Another embodiment comprises cyclosporin A, Dur-Em 117®, and Clary sage oil.

Another embodiment comprises cyclosporin A, Dur-Em 204®, and Clary sage oil.

Another embodiment comprises cyclosporin A, Dur-Em 207®, and Clary sage oil.

Another embodiment comprises cyclosporin A, Dur-Em 207E®, and Clary sage oil.

Another embodiment comprises cyclosporin A, Emerest 2704®, and Clary sage oil.

Another embodiment comprises cyclosporin A, Emerest 2712®, and Clary sage oil.

Another embodiment comprises cyclosporin A, Emerest 2715®, and Clary sage oil.

Another embodiment comprises cyclosporin A, Ethal EH-2®, and Clary sage oil.

Another embodiment comprises cyclosporin A, Ethal EH-5®, and Clary sage oil.

Another embodiment comprises cyclosporin A, Ethal OA-23®, and Clary sage oil.

Another embodiment comprises cyclosporin A, Ethosperse CA-20®, and Clary sage oil.

Another embodiment comprises cyclosporin A, Ethosperse LA-12®, and Clary sage oil.

Another embodiment comprises cyclosporin A, Ethosperse LA-23®, and Clary sage oil.

Another embodiment comprises cyclosporin A, Ethosperse LA-4®, and Clary sage oil.

Another embodiment comprises cyclosporin A, Ethox 3095®, and Clary sage oil.

Another embodiment comprises cyclosporin A, Ethox CO-16®, and Clary sage oil.

Another embodiment comprises cyclosporin A, Ethox CO-200®, and Clary sage oil.

Another embodiment comprises cyclosporin A, Ethox CO-25®, and Clary sage oil.

Another embodiment comprises cyclosporin A, Ethox CO-30®, and Clary sage oil.

Another embodiment comprises cyclosporin A, Ethox CO-36®, and Clary sage oil.

Another embodiment comprises cyclosporin A, Ethox CO-40®, and Clary sage oil.

Another embodiment comprises cyclosporin A, Ethox CO-5®, and Clary sage oil.

Another embodiment comprises cyclosporin A, Ethox DO-14®, and Clary sage oil.

Another embodiment comprises cyclosporin A, Ethox DO-9®, and Clary sage oil.

Another embodiment comprises cyclosporin A, Ethox MA-8®, and Clary sage oil.

Another embodiment comprises cyclosporin A, Ethox MI-14®, and Clary sage oil.

Another embodiment comprises cyclosporin A, Ethox ML-14®, and Clary sage oil.

Another embodiment comprises cyclosporin A, Ethox ML-5®, and Clary sage oil.

Another embodiment comprises cyclosporin A, Ethox MO-14®, and Clary sage oil.

Another embodiment comprises cyclosporin A, Ethox MO-9®, and Clary sage oil.

Another embodiment comprises cyclosporin A, Ethox MS-14®, and Clary sage oil.

Another embodiment comprises cyclosporin A, Ethox MS-23®, and Clary sage oil.

Another embodiment comprises cyclosporin A, Ethox MS-40®, and Clary sage oil.

Another embodiment comprises cyclosporin A, Ethox MS-8®, and Clary sage oil.

Another embodiment comprises cyclosporin A, Ethox TO-16®, and Clary sage oil.

Another embodiment comprises cyclosporin A, Ethox TO-8®, and Clary sage oil.

Another embodiment comprises cyclosporin A, Ethylan SN-120®, and Clary sage oil.

Another embodiment comprises cyclosporin A, Ethylan SN-70®, and Clary sage oil.

Another embodiment comprises cyclosporin A, Ethylan SN-90®, and Clary sage oil.

Another embodiment comprises cyclosporin A, Flexricin 13®, and Clary sage oil.

Another embodiment comprises cyclosporin A, Flexricin 15®, and Clary sage oil.

Another embodiment comprises cyclosporin A, Genapol 1261®, and Clary sage oil.

Another embodiment comprises cyclosporin A, Generol 122 E25®, and Clary sage oil.

Another embodiment comprises cyclosporin A, Glicepol 160®, and Clary sage oil.

Another embodiment comprises cyclosporin A, Glicepol 560®, and Clary sage oil.

Another embodiment comprises cyclosporin A, Glicepol GMS 20®, and Clary sage oil.

Another embodiment comprises cyclosporin A, Hetoxide BN-13®, and Clary sage oil.

Another embodiment comprises cyclosporin A, Hetoxide DNP-4®, and Clary sage oil.

Another embodiment comprises cyclosporin A, Hetoxide DNP-9.6®, and Clary sage oil.

Another embodiment comprises cyclosporin A, Hetoxide G-26®, and Clary sage oil.

Another embodiment comprises cyclosporin A, Hetoxide G-7®, and Clary sage oil.

Another embodiment comprises cyclosporin A, Hetoxide GT-20®, and Clary sage oil.

Another embodiment comprises cyclosporin A, Hetoxide HC-40®, and Clary sage oil.

Another embodiment comprises cyclosporin A, Hetoxol C-24®, and Clary sage oil.

Another embodiment comprises cyclosporin A, Hetoxol CA-10®, and Clary sage oil.

Another embodiment comprises cyclosporin A, Hetoxol CA-2®, and Clary sage oil.

Another embodiment comprises cyclosporin A, Hetoxol CA-20®, and Clary sage oil.

Another embodiment comprises cyclosporin A, Hetoxol CAWS®, and Clary sage oil.

Another embodiment comprises cyclosporin A, Hetoxol CD-3®, and Clary sage oil.

Another embodiment comprises cyclosporin A, Hetoxol CD-4®, and Clary sage oil.

Another embodiment comprises cyclosporin A, Hetoxol CS-15®, and Clary sage oil.

Another embodiment comprises cyclosporin A, Hetoxol CS-20®, and Clary sage oil.

Another embodiment comprises cyclosporin A, Hetoxol CS-20D®, and Clary sage oil.

Another embodiment comprises cyclosporin A, Hetoxol CS-25®, and Clary sage oil.

Another embodiment comprises cyclosporin A, Hetoxol CS-50®, and Clary sage oil.

Another embodiment comprises cyclosporin A, Hetoxol CS-9®, and Clary sage oil.

Another embodiment comprises cyclosporin A, Hetoxol CSA-15®, and Clary sage oil.

Another embodiment comprises cyclosporin A, Hetoxol D®, and Clary sage oil.

Another embodiment comprises cyclosporin A, Hetoxol G®, and Clary sage oil.

Another embodiment comprises cyclosporin A, Hetoxol J®, and Clary sage oil.

Another embodiment comprises cyclosporin A, Hetoxol L®, and Clary sage oil.

Another embodiment comprises cyclosporin A, Hetoxol L-23®, and Clary sage oil.

Another embodiment comprises cyclosporin A, Hetoxol L-4®, and Clary sage oil.

Another embodiment comprises cyclosporin A, Hetoxol L-9®, and Clary sage oil.

Another embodiment comprises cyclosporin A, Hetoxol LS-9®, and Clary sage oil.

Another embodiment comprises cyclosporin A, Hetoxol M-3®, and Clary sage oil.

Another embodiment comprises cyclosporin A, Hetoxol OA-10 Special®, and Clary sage oil.

Another embodiment comprises cyclosporin A, Hetoxol OA-20 Special®, and Clary sage oil.

Another embodiment comprises cyclosporin A, Hetoxol OA-3 Specia®, and Clary sage oil.

Another embodiment comprises cyclosporin A, Hetoxol OA-5 Special®, and Clary sage oil.

Another embodiment comprises cyclosporin A, Hetoxol OL-10®, and Clary sage oil.

Another embodiment comprises cyclosporin A, Hetoxol OL-2®, and Clary sage oil.

Another embodiment comprises cyclosporin A, Hetoxol OL-23®, and Clary sage oil.

Another embodiment comprises cyclosporin A, Hetoxol OL-4®, and Clary sage oil.

Another embodiment comprises cyclosporin A, Hetoxol OL-40®, and Clary sage oil.

Another embodiment comprises cyclosporin A, Hetoxol OL-5®, and Clary sage oil.

Another embodiment comprises cyclosporin A, Hetoxol PLA®, and Clary sage oil.

Another embodiment comprises cyclosporin A, Hetoxol SP-15®, and Clary sage oil.

Another embodiment comprises cyclosporin A, Hetoxol STA-10®, and Clary sage oil.

Another embodiment comprises cyclosporin A, Hetoxol STA-2®, and Clary sage oil.

Another embodiment comprises cyclosporin A, Hetoxol STA-30®, and Clary sage oil.

Another embodiment comprises cyclosporin A, Hetoxol TD-12®, and Clary sage oil.

Another embodiment comprises cyclosporin A, Hetoxol TD-18®, and Clary sage oil.

Another embodiment comprises cyclosporin A, Hetoxol TD-3®, and Clary sage oil.

Another embodiment comprises cyclosporin A, Hetoxol TD-6®, and Clary sage oil.

Another embodiment comprises cyclosporin A, Hetoxol TD-9®, and Clary sage oil.

Another embodiment comprises cyclosporin A, Hetoxol TDEP-15®, and Clary sage oil.

Another embodiment comprises cyclosporin A, Hetoxol TDEP-63®, and Clary sage oil.

Another embodiment comprises cyclosporin A, Iconol 24-12®, and Clary sage oil.

Another embodiment comprises cyclosporin A, Iconol 24-23®, and Clary sage oil.

Another embodiment comprises cyclosporin A, Iconol 24-4®, and Clary sage oil.

Another embodiment comprises cyclosporin A, Iconol DA-4®, and Clary sage oil.

Another embodiment comprises cyclosporin A, Iconol DA-6®, and Clary sage oil.

Another embodiment comprises cyclosporin A, Iconol TDA-10®, and Clary sage oil.

Another embodiment comprises cyclosporin A, Iconol TDA-3®, and Clary sage oil.

Another embodiment comprises cyclosporin A, Iconol TDA-6®, and Clary sage oil.

Another embodiment comprises cyclosporin A, Iconol TDA-8-90%®, and Clary sage oil.

Another embodiment comprises cyclosporin A, Iconol TDA-9®, and Clary sage oil.

Another embodiment comprises cyclosporin A, Igepal CA-210®, and Clary sage oil.

Another embodiment comprises cyclosporin A, Igepal CA-407®, and Clary sage oil.

Another embodiment comprises cyclosporin A, Igepal CA-420®, and Clary sage oil.

Another embodiment comprises cyclosporin A, Igepal CA-520®, and Clary sage oil.

Another embodiment comprises cyclosporin A, Igepal CA-620®, and Clary sage oil.

Another embodiment comprises cyclosporin A, Igepal CA-630®, and Clary sage oil.

Another embodiment comprises cyclosporin A, Igepal CA-720®, and Clary sage oil.

Another embodiment comprises cyclosporin A, Igepal CA-877®, and Clary sage oil.

Another embodiment comprises cyclosporin A, Igepal CA-887®, and Clary sage oil.

Another embodiment comprises cyclosporin A, Igepal CA-897®, and Clary sage oil.

Another embodiment comprises cyclosporin A, Igepal CO-210®, and Clary sage oil.

Another embodiment comprises cyclosporin A, Igepal CO-430®, and Clary sage oil.

Another embodiment comprises cyclosporin A, Igepal CO-520®, and Clary sage oil.

Another embodiment comprises cyclosporin A, Igepal CO-530®, and Clary sage oil.

Another embodiment comprises cyclosporin A, Igepal CO-610®, and Clary sage oil.

Another embodiment comprises cyclosporin A, Igepal CO-630®, and Clary sage oil.

Another embodiment comprises cyclosporin A, Igepal CO-630 Special®, and Clary sage oil.

Another embodiment comprises cyclosporin A, Igepal CO-660®, and Clary sage oil.

Another embodiment comprises cyclosporin A, Igepal CO-710®, and Clary sage oil.

Another embodiment comprises cyclosporin A, Igepal CO-720®, and Clary sage oil.

Another embodiment comprises cyclosporin A, Igepal CO-730®, and Clary sage oil.

Another embodiment comprises cyclosporin A, Igepal CO-738®, and Clary sage oil.

Another embodiment comprises cyclosporin A, Igepal CO-850®, and Clary sage oil.

Another embodiment comprises cyclosporin A, Igepal CO-880®, and Clary sage oil.

Another embodiment comprises cyclosporin A, Igepal CO-887®, and Clary sage oil.

Another embodiment comprises cyclosporin A, Igepal CO-890®, and Clary sage oil.

Another embodiment comprises cyclosporin A, Igepal CO-897®, and Clary sage oil.

Another embodiment comprises cyclosporin A, Igepal CO-970®, and Clary sage oil.

Another embodiment comprises cyclosporin A, Igepal CO-977®, and Clary sage oil.

Another embodiment comprises cyclosporin A, Igepal CO-987®, and Clary sage oil.

Another embodiment comprises cyclosporin A, Igepal CO-990 FLK®, and Clary sage oil.

Another embodiment comprises cyclosporin A, Igepal CO-997®, and Clary sage oil.

Another embodiment comprises cyclosporin A, Igepal DM-430®, and Clary sage oil.

Another embodiment comprises cyclosporin A, Igepal DM-530®, and Clary sage oil.

Another embodiment comprises cyclosporin A, Igepal DM-710®, and Clary sage oil.

Another embodiment comprises cyclosporin A, Igepal DM-730®, and Clary sage oil.

Another embodiment comprises cyclosporin A, Igepal DM-880®, and Clary sage oil.

Another embodiment comprises cyclosporin A, Igepal DM-970 FLK®, and Clary sage oil.

Another embodiment comprises cyclosporin A, Igepal OD-410®, and Clary sage oil.

Another embodiment comprises cyclosporin A, Igepal RC-520®, and Clary sage oil.

Another embodiment comprises cyclosporin A, Igepal RC-620®, and Clary sage oil.

Another embodiment comprises cyclosporin A, Igepal RC-630®, and Clary sage oil.

Another embodiment comprises cyclosporin A, Lexemul CS-20®, and Clary sage oil.

Another embodiment comprises cyclosporin A, Lipo GMS 450®, and Clary sage oil.

Another embodiment comprises cyclosporin A, Lipo Polyglycol 1000®, and Clary sage oil.

Another embodiment comprises cyclosporin A, Lipo Polyglycol 200®, and Clary sage oil.

Another embodiment comprises cyclosporin A, Lipo Polyglycol 300®, and Clary sage oil.

Another embodiment comprises cyclosporin A, Lipo Polyglycol 3350®, and Clary sage oil.

Another embodiment comprises cyclosporin A, Lipo Polyglycol 400®, and Clary sage oil.

Another embodiment comprises cyclosporin A, Lipo Polyglycol 600®, and Clary sage oil.

Another embodiment comprises cyclosporin A, Lipocol C-10®, and Clary sage oil.

Another embodiment comprises cyclosporin A, Lipocol C-2®, and Clary sage oil.

Another embodiment comprises cyclosporin A, Lipocol C-20®, and Clary sage oil.

Another embodiment comprises cyclosporin A, Lipocol L-12®, and Clary sage oil.

Another embodiment comprises cyclosporin A, Lipocol L-23®, and Clary sage oil.

Another embodiment comprises cyclosporin A, Lipocol L-4®, and Clary sage oil.

Another embodiment comprises cyclosporin A, Lipocol O-10®, and Clary sage oil.

Another embodiment comprises cyclosporin A, Lipocol O-2®, and Clary sage oil.

Another embodiment comprises cyclosporin A, Lipocol O-20®, and Clary sage oil.

Another embodiment comprises cyclosporin A, Lipocol O-5®, and Clary sage oil.

Another embodiment comprises cyclosporin A, Lipocol S-10®, and Clary sage oil.

Another embodiment comprises cyclosporin A, Lipocol S-2®, and Clary sage oil.

Another embodiment comprises cyclosporin A, Lipocol S-20®, and Clary sage oil.

Another embodiment comprises cyclosporin A, Lipocol SC-15®, and Clary sage oil.

Another embodiment comprises cyclosporin A, Lipocol SC-20®, and Clary sage oil.

Another embodiment comprises cyclosporin A, Lipocol SC-4®, and Clary sage oil.

Another embodiment comprises cyclosporin A, Lipocol TD-12®, and Clary sage oil.

Another embodiment comprises cyclosporin A, Lumulse 40-O K®, and Clary sage oil.

Another embodiment comprises cyclosporin A, Lumulse 42-O K®, and Clary sage oil.

Another embodiment comprises cyclosporin A, Lumulse GMT-40®, and Clary sage oil.

Another embodiment comprises cyclosporin A, Lumulse PEG 1450®, and Clary sage oil.

Another embodiment comprises cyclosporin A, Lumulse PEG 1450 NF®, and Clary sage oil.

Another embodiment comprises cyclosporin A, Lumulse PEG 8000®, and Clary sage oil.

Another embodiment comprises cyclosporin A, Lumulse PGO®, and Clary sage oil.

Another embodiment comprises cyclosporin A, Lumulse POE (2) Oleyl Amine®, and Clary sage oil.

Another embodiment comprises cyclosporin A, Magrabar PGO-104®, and Clary sage oil.

Another embodiment comprises cyclosporin A, Mapeg 200 ML®, and Clary sage oil.

Another embodiment comprises cyclosporin A, Mapeg 400 MO®, and Clary sage oil.

Another embodiment comprises cyclosporin A, Mapeg 400 MOT®, and Clary sage oil.

Another embodiment comprises cyclosporin A, Mapeg 400DO®, and Clary sage oil.

Another embodiment comprises cyclosporin A, Mapeg 400DOT®, and Clary sage oil.

Another embodiment comprises cyclosporin A, Mapeg 600DOT®, and Clary sage oil.

Another embodiment comprises cyclosporin A, Marlox B 24/80®, and Clary sage oil.

Another embodiment comprises cyclosporin A, Marlox FK 86®, and Clary sage oil.

Another embodiment comprises cyclosporin A, Marlox MO 154®, and Clary sage oil.

Another embodiment comprises cyclosporin A, Mazox CDA CG®, and Clary sage oil.

Another embodiment comprises cyclosporin A, Monoglycerides Glyceryl Monestearate®, and Clary sage oil.

Another embodiment comprises cyclosporin A, Myrj 45®, and Clary sage oil.

Another embodiment comprises cyclosporin A, Myrj 52®, and Clary sage oil.

Another embodiment comprises cyclosporin A, Myrj 52S®, and Clary sage oil.

Another embodiment comprises cyclosporin A, Myrj 53®, and Clary sage oil.

Another embodiment comprises cyclosporin A, Myrj 59®, and Clary sage oil.

Another embodiment comprises cyclosporin A, Norfox NP-12®, and Clary sage oil.

Another embodiment comprises cyclosporin A, Norfox NP-4®, and Clary sage oil.

Another embodiment comprises cyclosporin A, Norfox NP-6®, and Clary sage oil.

Another embodiment comprises cyclosporin A, Norfox NP-887®, and Clary sage oil.

Another embodiment comprises cyclosporin A, Norfox NP-9®, and Clary sage oil.

Another embodiment comprises cyclosporin A, Norfox NP-977®, and Clary sage oil.

Another embodiment comprises cyclosporin A, Norfox OP-100®, and Clary sage oil.

Another embodiment comprises cyclosporin A, Norfox OP-102®, and Clary sage oil.

Another embodiment comprises cyclosporin A, Norfox OP-114®, and Clary sage oil.

Another embodiment comprises cyclosporin A, Norfox OP-45®, and Clary sage oil.

Another embodiment comprises cyclosporin A, Pegosperse 100-L®, and Clary sage oil.

Another embodiment comprises cyclosporin A, Pegosperse 100-O®, and Clary sage oil.

Another embodiment comprises cyclosporin A, Pegosperse 100-S®, and Clary sage oil.

Another embodiment comprises cyclosporin A, Pegosperse 1500-MS®, and Clary sage oil.

Another embodiment comprises cyclosporin A, Pegosperse 1750-MS®, and Clary sage oil.

Another embodiment comprises cyclosporin A, Pegosperse 200 DL®, and Clary sage oil.

Another embodiment comprises cyclosporin A, Pegosperse 200-ML®, and Clary sage oil.

Another embodiment comprises cyclosporin A, Pegosperse 400-DL®, and Clary sage oil.

Another embodiment comprises cyclosporin A, Pegosperse 400-DO®, and Clary sage oil.

Another embodiment comprises cyclosporin A, Pegosperse 400-DS®, and Clary sage oil.

Another embodiment comprises cyclosporin A, Pegosperse 400-ML®, and Clary sage oil.

Another embodiment comprises cyclosporin A, Pegosperse 400-MO®, and Clary sage oil.

Another embodiment comprises cyclosporin A, Pegosperse 400-MOT®, and Clary sage oil.

Another embodiment comprises cyclosporin A, Pegosperse 400-MS®, and Clary sage oil.

Another embodiment comprises cyclosporin A, Pegosperse 50-DS®, and Clary sage oil.

Another embodiment comprises cyclosporin A, Pegosperse 50-MS®, and Clary sage oil.

Another embodiment comprises cyclosporin A, Pegosperse 600-ML®, and Clary sage oil.

Another embodiment comprises cyclosporin A, Pegosperse 600-MS®, and Clary sage oil.

Another embodiment comprises cyclosporin A, Plurafac A-38®, and Clary sage oil.

Another embodiment comprises cyclosporin A, Plurafac B-25-5®, and Clary sage oil.

Another embodiment comprises cyclosporin A, Plurafac B-26®, and Clary sage oil.

Another embodiment comprises cyclosporin A, Plurafac D-25®, and Clary sage oil.

Another embodiment comprises cyclosporin A, Plurafac LF 131®, and Clary sage oil.

Another embodiment comprises cyclosporin A, Plurafac LF 4030®, and Clary sage oil.

Another embodiment comprises cyclosporin A, Plurafac LF 7000®, and Clary sage oil.

Another embodiment comprises cyclosporin A, Plurafac RA-20®, and Clary sage oil.

Another embodiment comprises cyclosporin A, Plurafac RA-30®, and Clary sage oil.

Another embodiment comprises cyclosporin A, Plurafac RA-40®, and Clary sage oil.

Another embodiment comprises cyclosporin A, Pluronic 10R5®, and Clary sage oil.

Another embodiment comprises cyclosporin A, Pluronic 17R2®, and Clary sage oil.

Another embodiment comprises cyclosporin A, Pluronic 17R4®, and Clary sage oil.

Another embodiment comprises cyclosporin A, Pluronic 25R2®, and Clary sage oil.

Another embodiment comprises cyclosporin A, Pluronic 25R4®, and Clary sage oil.

Another embodiment comprises cyclosporin A, Pluronic 31R1®, and Clary sage oil.

Another embodiment comprises cyclosporin A, Pluronic F108®, and Clary sage oil.

Another embodiment comprises cyclosporin A, Pluronic F127®, and Clary sage oil.

Another embodiment comprises cyclosporin A, Pluronic F38®, and Clary sage oil.

Another embodiment comprises cyclosporin A, Pluronic F68®, and Clary sage oil.

Another embodiment comprises cyclosporin A, Pluronic F68LF®, and Clary sage oil.

Another embodiment comprises cyclosporin A, Pluronic F77®, and Clary sage oil.

Another embodiment comprises cyclosporin A, Pluronic F87®, and Clary sage oil.

Another embodiment comprises cyclosporin A, Pluronic F88®, and Clary sage oil.

Another embodiment comprises cyclosporin A, Pluronic F98®, and Clary sage oil.

Another embodiment comprises cyclosporin A, Pluronic L10®, and Clary sage oil.

Another embodiment comprises cyclosporin A, Pluronic L101®, and Clary sage oil.

Another embodiment comprises cyclosporin A, Pluronic L121®, and Clary sage oil.

Another embodiment comprises cyclosporin A, Pluronic L31®, and Clary sage oil.

Another embodiment comprises cyclosporin A, Pluronic L35®, and Clary sage oil.

Another embodiment comprises cyclosporin A, Pluronic L43®, and Clary sage oil.

Another embodiment comprises cyclosporin A, Pluronic L44®, and Clary sage oil.

Another embodiment comprises cyclosporin A, Pluronic L61®, and Clary sage oil.

Another embodiment comprises cyclosporin A, Pluronic L62®, and Clary sage oil.

Another embodiment comprises cyclosporin A, Pluronic L62D®, and Clary sage oil.

Another embodiment comprises cyclosporin A, Pluronic L62LF®, and Clary sage oil.

Another embodiment comprises cyclosporin A, Pluronic L64®, and Clary sage oil.

Another embodiment comprises cyclosporin A, Pluronic L81®, and Clary sage oil.

Another embodiment comprises cyclosporin A, Pluronic L92®, and Clary sage oil.

Another embodiment comprises cyclosporin A, Pluronic P103®, and Clary sage oil.

Another embodiment comprises cyclosporin A, Pluronic P104®, and Clary sage oil.

Another embodiment comprises cyclosporin A, Pluronic P105®, and Clary sage oil.

Another embodiment comprises cyclosporin A, Pluronic P123®, and Clary sage oil.

Another embodiment comprises cyclosporin A, Pluronic P65®, and Clary sage oil.

Another embodiment comprises cyclosporin A, Pluronic P84®, and Clary sage oil.

Another embodiment comprises cyclosporin A, Pluronic P85®, and Clary sage oil.

Another embodiment comprises cyclosporin A, Polyaldo 10-2-P®, and Clary sage oil.

Another embodiment comprises cyclosporin A, Polyaldo DGDO®, and Clary sage oil.

Another embodiment comprises cyclosporin A, Polyaldo HGDS®, and Clary sage oil.

Another embodiment comprises cyclosporin A, Polyaldo TGMS®, and Clary sage oil.

Another embodiment comprises cyclosporin A, Protegin V®, and Clary sage oil.

Another embodiment comprises cyclosporin A, Protegin W®, and Clary sage oil.

Another embodiment comprises cyclosporin A, Protegin WX®, and Clary sage oil.

Another embodiment comprises cyclosporin A, Protegin XV®, and Clary sage oil.

Another embodiment comprises cyclosporin A, Rhodasurf BC-420®, and Clary sage oil.

Another embodiment comprises cyclosporin A, Rhodasurf BC-610®, and Clary sage oil.

Another embodiment comprises cyclosporin A, Rhodasurf BC-630®, and Clary sage oil.

Another embodiment comprises cyclosporin A, Rhodasurf BC-720®, and Clary sage oil.

Another embodiment comprises cyclosporin A, Rhodasurf BC-840®, and Clary sage oil.

Another embodiment comprises cyclosporin A, Rhodasurf L 1®, and Clary sage oil.

Another embodiment comprises cyclosporin A, Rhodasurf L 12®, and Clary sage oil.

Another embodiment comprises cyclosporin A, Rhodasurf L 2®, and Clary sage oil.

Another embodiment comprises cyclosporin A, Rhodasurf L 20®, and Clary sage oil.

Another embodiment comprises cyclosporin A, Rhodasurf L 3®, and Clary sage oil.

Another embodiment comprises cyclosporin A, Rhodasurf L 30®, and Clary sage oil.

Another embodiment comprises cyclosporin A, Rhodasurf L 4®, and Clary sage oil.

Another embodiment comprises cyclosporin A, Rhodasurf L 50®, and Clary sage oil.

Another embodiment comprises cyclosporin A, Rhodasurf L 7/90®, and Clary sage oil.

Another embodiment comprises cyclosporin A, Rhodasurf LA-3®, and Clary sage oil.

Another embodiment comprises cyclosporin A, Rhodasurf LA-7®, and Clary sage oil.

Another embodiment comprises cyclosporin A, Rhodasurf LAN-23®, and Clary sage oil.

Another embodiment comprises cyclosporin A, Rhodasurf ON-870®, and Clary sage oil.

Another embodiment comprises cyclosporin A, Rhodasurf ON-877®, and Clary sage oil.

Another embodiment comprises cyclosporin A, Rhodasurf TDA-8/5®, and Clary sage oil.

Another embodiment comprises cyclosporin A, Ritapro 100®, and Clary sage oil.

Another embodiment comprises cyclosporin A, Ritapro 165®, and Clary sage oil.

Another embodiment comprises cyclosporin A, Ritapro 200®, and Clary sage oil.

Another embodiment comprises cyclosporin A, Ritapro 300®, and Clary sage oil.

Another embodiment comprises cyclosporin A, Ritoleth 10®, and Clary sage oil.

Another embodiment comprises cyclosporin A, Ritoleth 2®, and Clary sage oil.

Another embodiment comprises cyclosporin A, Ritoleth 20®, and Clary sage oil.

Another embodiment comprises cyclosporin A, Ritoleth 5®, and Clary sage oil.

Another embodiment comprises cyclosporin A, Santone 10-10-0®, and Clary sage oil.

Another embodiment comprises cyclosporin A, Santone 3-1-S XTR®, and Clary sage oil.

Another embodiment comprises cyclosporin A, Santone 3-1-SH®, and Clary sage oil.

Another embodiment comprises cyclosporin A, Santone 8-1-O®, and Clary sage oil.

Another embodiment comprises cyclosporin A, Solulan 16®, and Clary sage oil.

Another embodiment comprises cyclosporin A, Solulan 75®, and Clary sage oil.

Another embodiment comprises cyclosporin A, Solulan C-24®, and Clary sage oil.

Another embodiment comprises cyclosporin A, Solulan L-575®, and Clary sage oil.

Another embodiment comprises cyclosporin A, Standamox CAW®, and Clary sage oil.

Another embodiment comprises cyclosporin A, Surfine AZI-A®, and Clary sage oil.

Another embodiment comprises cyclosporin A, Surfonic CO-15®, and Clary sage oil.

Another embodiment comprises cyclosporin A, Surfonic CO-25®, and Clary sage oil.

Another embodiment comprises cyclosporin A, Surfonic CO-30®, and Clary sage oil.

Another embodiment comprises cyclosporin A, Surfonic CO-36®, and Clary sage oil.

Another embodiment comprises cyclosporin A, Surfonic CO-42®, and Clary sage oil.

Another embodiment comprises cyclosporin A, Surfonic E400-MO®, and Clary sage oil.

Another embodiment comprises cyclosporin A, Surfonic L12-3®, and Clary sage oil.

Another embodiment comprises cyclosporin A, Surfonic L12-6®, and Clary sage oil.

Another embodiment comprises cyclosporin A, Surfonic L12-8®, and Clary sage oil.

Another embodiment comprises cyclosporin A, Surfonic L24-12®, and Clary sage oil.

Another embodiment comprises cyclosporin A, Surfonic L24-2®, and Clary sage oil.

Another embodiment comprises cyclosporin A, Surfonic L24-22®, and Clary sage oil.

Another embodiment comprises cyclosporin A, Surfonic L24-3®, and Clary sage oil.

Another embodiment comprises cyclosporin A, Surfonic L24-4®, and Clary sage oil.

Another embodiment comprises cyclosporin A, Surfonic L24-5®, and Clary sage oil.

Another embodiment comprises cyclosporin A, Surfonic L24-7®, and Clary sage oil.

Another embodiment comprises cyclosporin A, Surfonic L24-7.1®, and Clary sage oil.

Another embodiment comprises cyclosporin A, Surfonic L24-9®, and Clary sage oil.

Another embodiment comprises cyclosporin A, Surfonic LF-17®, and Clary sage oil.

Another embodiment comprises cyclosporin A, Surfonic LF-18®, and Clary sage oil.

Another embodiment comprises cyclosporin A, Surfonic LF-37®, and Clary sage oil.

Another embodiment comprises cyclosporin A, Surfonic LF-41®, and Clary sage oil.

Another embodiment comprises cyclosporin A, Surfonic LF-42®, and Clary sage oil.

Another embodiment comprises cyclosporin A, Surfonic LF-47®, and Clary sage oil.

Another embodiment comprises cyclosporin A, Surfonic LF-68®, and Clary sage oil.

Another embodiment comprises cyclosporin A, Surfonic N-10®, and Clary sage oil.

Another embodiment comprises cyclosporin A, Surfonic N-100®, and Clary sage oil.

Another embodiment comprises cyclosporin A, Surfonic N-102®, and Clary sage oil.

Another embodiment comprises cyclosporin A, Surfonic N-120®, and Clary sage oil.

Another embodiment comprises cyclosporin A, Surfonic N-150®, and Clary sage oil.

Another embodiment comprises cyclosporin A, Surfonic N-200®, and Clary sage oil.

Another embodiment comprises cyclosporin A, Surfonic N-300®, and Clary sage oil.

Another embodiment comprises cyclosporin A, Surfonic N-31.5®, and Clary sage oil.

Another embodiment comprises cyclosporin A, Surlonic N-40®, and Clary sage oil.

Another embodiment comprises cyclosporin A, Surfonic N-400®, and Clary sage oil.

Another embodiment comprises cyclosporin A, Surfonic N-60®, and Clary sage oil.

Another embodiment comprises cyclosporin A, Surfonic N-85®, and Clary sage oil.

Another embodiment comprises cyclosporin A, Surfonic N-95®, and Clary sage oil.

Another embodiment comprises cyclosporin A, Surfonic NB-18®, and Clary sage oil.

Another embodiment comprises cyclosporin A, Surfonic NB-23®, and Clary sage oil.

Another embodiment comprises cyclosporin A, Surfonic NB-25®, and Clary sage oil.

Another embodiment comprises cyclosporin A, Surfonic NB-307®, and Clary sage oil.

Another embodiment comprises cyclosporin A, Surfonic NB-31®, and Clary sage oil.

Another embodiment comprises cyclosporin A, Surfonic NB-41®, and Clary sage oil.

Another embodiment comprises cyclosporin A, Surlonic NB-43®, and Clary sage oil.

Another embodiment comprises cyclosporin A, Surfonic OP-100®, and Clary sage oil.

Another embodiment comprises cyclosporin A, Surfonic OP-120®, and Clary sage oil.

Another embodiment comprises cyclosporin A, Surfonic OP-15®, and Clary sage oil.

Another embodiment comprises cyclosporin A, Surfonic OP-35®, and Clary sage oil.

Another embodiment comprises cyclosporin A, Surfonic OP-70®, and Clary sage oil.

Another embodiment comprises cyclosporin A, Surfonic P1®, and Clary sage oil.

Another embodiment comprises cyclosporin A, Surfonic P3®, and Clary sage oil.

Another embodiment comprises cyclosporin A, Surfonic P5®, and Clary sage oil.

Another embodiment comprises cyclosporin A, Surfonic P6®, and Clary sage oil.

Another embodiment comprises cyclosporin A, Surfonic POA-17R2®, and Clary sage oil.

Another embodiment comprises cyclosporin A, Surfonic POA-25R2®, and Clary sage oil.

Another embodiment comprises cyclosporin A, Surfonic TDA-11®, and Clary sage oil.

Another embodiment comprises cyclosporin A, Surfonic TDA-3B®, and Clary sage oil.

Another embodiment comprises cyclosporin A, Surfonic TDA-6®, and Clary sage oil.

Another embodiment comprises cyclosporin A, Surfonic TDA-6/88®, and Clary sage oil.

Another embodiment comprises cyclosporin A, Surfonic TDA-8®, and Clary sage oil.

Another embodiment comprises cyclosporin A, Surfonic TDA-8/90®, and Clary sage oil.

Another embodiment comprises cyclosporin A, Surfonic TDA-9®, and Clary sage oil.

Another embodiment comprises cyclosporin A, Surfonic TX-CCN®, and Clary sage oil.

Another embodiment comprises cyclosporin A, Tagat CH 40®, and Clary sage oil.

Another embodiment comprises cyclosporin A, Tagat CH 60®, and Clary sage oil.

Another embodiment comprises cyclosporin A, Tagat L 2®, and Clary sage oil.

Another embodiment comprises cyclosporin A, Tagat S®, and Clary sage oil.

Another embodiment comprises cyclosporin A, Tagat S 2®, and Clary sage oil.

Another embodiment comprises cyclosporin A, Tally 100 Plus®, and Clary sage oil.

Another embodiment comprises cyclosporin A, T-Det A106®, and Clary sage oil.

Another embodiment comprises cyclosporin A, T-Det A109®, and Clary sage oil.

Another embodiment comprises cyclosporin A, T-Det A136®, and Clary sage oil.

Another embodiment comprises cyclosporin A, T-Det A139®, and Clary sage oil.

Another embodiment comprises cyclosporin A, T-Det A2412®, and Clary sage oil.

Another embodiment comprises cyclosporin A, T-Det A243®, and Clary sage oil.

Another embodiment comprises cyclosporin A, T-Det A247®, and Clary sage oil.

Another embodiment comprises cyclosporin A, T-Det A249®, and Clary sage oil.

Another embodiment comprises cyclosporin A, T-Det A467®, and Clary sage oil.

Another embodiment comprises cyclosporin A, T-Det COE®, and Clary sage oil.

Another embodiment comprises cyclosporin A, T-Det DD-10®, and Clary sage oil.

Another embodiment comprises cyclosporin A, T-Det DD-5®, and Clary sage oil.

Another embodiment comprises cyclosporin A, T-Det DD-7®, and Clary sage oil.

Another embodiment comprises cyclosporin A, T-Det EPO-104®, and Clary sage oil.

Another embodiment comprises cyclosporin A, T-Det EPO-61®, and Clary sage oil.

Another embodiment comprises cyclosporin A, T-Det EPO-62®, and Clary sage oil.

Another embodiment comprises cyclosporin A, T-Det EPO-64®, and Clary sage oil.

Another embodiment comprises cyclosporin A, T-Det N-1.5®, and Clary sage oil.

Another embodiment comprises cyclosporin A, T-Det N-10.5®, and Clary sage oil.

Another embodiment comprises cyclosporin A, T-Det N-100®, and Clary sage oil.

Another embodiment comprises cyclosporin A, T-Det N-1007®, and Clary sage oil.

Another embodiment comprises cyclosporin A, T-Det N-12®, and Clary sage oil.

Another embodiment comprises cyclosporin A, T-Det N-14®, and Clary sage oil.

Another embodiment comprises cyclosporin A, T-Det N-20®, and Clary sage oil.

Another embodiment comprises cyclosporin A, T-Det N-30®, and Clary sage oil.

Another embodiment comprises cyclosporin A, T-Det N-307®, and Clary sage oil.

Another embodiment comprises cyclosporin A, T-Det N-4®, and Clary sage oil.

Another embodiment comprises cyclosporin A, T-Det N-40®, and Clary sage oil.

Another embodiment comprises cyclosporin A, T-Det N-407®, and Clary sage oil.

Another embodiment comprises cyclosporin A, T-Det N-50®, and Clary sage oil.

Another embodiment comprises cyclosporin A, T-Det N-507®, and Clary sage oil.

Another embodiment comprises cyclosporin A, T-Det N-6®, and Clary sage oil.

Another embodiment comprises cyclosporin A, T-Det N-70®, and Clary sage oil.

Another embodiment comprises cyclosporin A, T-Det N-8®, and Clary sage oil.

Another embodiment comprises cyclosporin A, T-Det N-9.5®, and Clary sage oil.

Another embodiment comprises cyclosporin A, T-Det O-12®, and Clary sage oil.

Another embodiment comprises cyclosporin A, T-Det O-165®, and Clary sage oil.

Another embodiment comprises cyclosporin A, T-Det O-307®, and Clary sage oil.

Another embodiment comprises cyclosporin A, T-Det O-40®, and Clary sage oil.

Another embodiment comprises cyclosporin A, T-Det O-407®, and Clary sage oil.

Another embodiment comprises cyclosporin A, T-Det O-6®, and Clary sage oil.

Another embodiment comprises cyclosporin A, T-Det O-8®, and Clary sage oil.

Another embodiment comprises cyclosporin A, T-Det O-9®, and Clary sage oil.

Another embodiment comprises cyclosporin A, Tegin®, and Clary sage oil.

Another embodiment comprises cyclosporin A, Tergitol 15-S-12®, and Clary sage oil.

Another embodiment comprises cyclosporin A, Tergitol 15-S-15®, and Clary sage oil.

Another embodiment comprises cyclosporin A, Tergitol 15-S-20®, and Clary sage oil.

Another embodiment comprises cyclosporin A, Tergitol 15-S-20 (80AQ)®, and Clary sage oil.

Another embodiment comprises cyclosporin A, Tergitol 15-S-3®, and Clary sage oil.

Another embodiment comprises cyclosporin A, Tergitol 15-S-30®, and Clary sage oil.

Another embodiment comprises cyclosporin A, Tergitol 15-S-40®, and Clary sage oil.

Another embodiment comprises cyclosporin A, Tergitol 15-S-40 (70AQ)®, and Clary sage oil.

Another embodiment comprises cyclosporin A, Tergitol 15-S-5®, and Clary sage oil.

Another embodiment comprises cyclosporin A, Tergitol 15-S-7®, and Clary sage oil.

Another embodiment comprises cyclosporin A, Tergitol 15-S-9®, and Clary sage oil.

Another embodiment comprises cyclosporin A, Tergitol L-101 ®, and Clary sage oil.

Another embodiment comprises cyclosporin A, Tergitol L-61®, and Clary sage oil.

Another embodiment comprises cyclosporin A, Tergitol L-62®, and Clary sage oil.

Another embodiment comprises cyclosporin A, Tergitol L-64®, and Clary sage oil.

Another embodiment comprises cyclosporin A, Tergitol L-81®, and Clary sage oil.

Another embodiment comprises cyclosporin A, Tergitol NP-10®, and Clary sage oil.

Another embodiment comprises cyclosporin A, Tergitol NP-11®, and Clary sage oil.

Another embodiment comprises cyclosporin A, Tergitol NP-12®, and Clary sage oil.

Another embodiment comprises cyclosporin A, Tergitol NP-13®, and Clary sage oil.

Another embodiment comprises cyclosporin A, Tergitol NP-15®, and Clary sage oil.

Another embodiment comprises cyclosporin A, Tergitol NP-30®, and Clary sage oil.

Another embodiment comprises cyclosporin A, Tergitol NP-30 (70 AQ)®, and Clary sage oil.

Another embodiment comprises cyclosporin A, Tergitol NP-4®, and Clary sage oil.

Another embodiment comprises cyclosporin A, Tergitol NP-40®, and Clary sage oil.

Another embodiment comprises cyclosporin A, Tergitol NP-40 (70 AQ)®, and Clary sage oil.

Another embodiment comprises cyclosporin A, Tergitol NP-50 (70 AQ)®, and Clary sage oil.

Another embodiment comprises cyclosporin A, Tergitol NP-55 (70 AQ)®, and Clary sage oil.

Another embodiment comprises cyclosporin A, Tergitol NP-6®, and Clary sage oil.

Another embodiment comprises cyclosporin A, Tergitol NP-7®, and Clary sage oil.

Another embodiment comprises cyclosporin A, Tergitol NP-70 (70 AQ)®, and Clary sage oil.

Another embodiment comprises cyclosporin A, Tergitol NP-8®, and Clary sage oil.

Another embodiment comprises cyclosporin A, Tergitol NP-9®, and Clary sage oil.

Another embodiment comprises cyclosporin A, Tergitol NP-9.5®, and Clary sage oil.

Another embodiment comprises cyclosporin A, Tergitol TMN-10®, and Clary sage oil.

Another embodiment comprises cyclosporin A, Tergitol TMN-3®, and Clary sage oil.

Another embodiment comprises cyclosporin A, Tergitol TMN-6®, and Clary sage oil.

Another embodiment comprises cyclosporin A, Tergitol XD®, and Clary sage oil.

Another embodiment comprises cyclosporin A, Tergitol XH®, and Clary sage oil.

Another embodiment comprises cyclosporin A, Tergitol XJ®, and Clary sage oil.

Another embodiment comprises cyclosporin A, Tetronic 1107®, and Clary sage oil.

Another embodiment comprises cyclosporin A, Tetronic 1307®, and Clary sage oil.

Another embodiment comprises cyclosporin A, Tetronic 150R1®, and Clary sage oil.

Another embodiment comprises cyclosporin A, Tetronic 304®, and Clary sage oil.

Another embodiment comprises cyclosporin A, Tetronic 701®, and Clary sage oil.

Another embodiment comprises cyclosporin A, Tetronic 901®, and Clary sage oil.

Another embodiment comprises cyclosporin A, Tetronic 904®, and Clary sage oil.

Another embodiment comprises cyclosporin A, Tetronic 908®, and Clary sage oil.

Another embodiment comprises cyclosporin A, Tetronic 90R4®, and Clary sage oil.

Another embodiment comprises cyclosporin A, Tomadol 1-3®, and Clary sage oil.

Another embodiment comprises cyclosporin A, Tomadol 1-5®, and Clary sage oil.

Another embodiment comprises cyclosporin A, Tomadol 1-7®, and Clary sage oil.

Another embodiment comprises cyclosporin A, Tomadol 23-1®, and Clary sage oil.

Another embodiment comprises cyclosporin A, Tomadol 23-3®, and Clary sage oil.

Another embodiment comprises cyclosporin A, Tomadol 23-5®, and Clary sage oil.

Another embodiment comprises cyclosporin A, Tomadol 23-6.5®, and Clary sage oil.

Another embodiment comprises cyclosporin A, Tomadol 25-12®, and Clary sage oil.

Another embodiment comprises cyclosporin A, Tomadol 25-3®, and Clary sage oil.

Another embodiment comprises cyclosporin A, Tomadol 25-7®, and Clary sage oil.

Another embodiment comprises cyclosporin A, Tomadol 25-9®, and Clary sage oil.

Another embodiment comprises cyclosporin A, Tomadol 45-13®, and Clary sage oil.

Another embodiment comprises cyclosporin A, Tomadol 45-2.5®, and Clary sage oil.

Another embodiment comprises cyclosporin A, Tomadol 45-7®, and Clary sage oil.

Another embodiment comprises cyclosporin A, Tomadol 91-2.5®, and Clary sage oil.

Another embodiment comprises cyclosporin A, Tomadol 91-6®, and Clary sage oil.

Another embodiment comprises cyclosporin A, Tomadol 91-8®, and Clary sage oil.

Another embodiment comprises cyclosporin A, Triton X-100®, and Clary sage oil.

Another embodiment comprises cyclosporin A, Triton X-102®, and Clary sage oil.

Another embodiment comprises cyclosporin A, Triton X-114®, and Clary sage oil.

Another embodiment comprises cyclosporin A, Triton X-15®, and Clary sage oil.

Another embodiment comprises cyclosporin A, Triton X-165 (70%)®, and Clary sage oil.

Another embodiment comprises cyclosporin A, Triton X-207®, and Clary sage oil.

Another embodiment comprises cyclosporin A, Triton X-305 (70%)®, and Clary sage oil.

Another embodiment comprises cyclosporin A, Triton X-35®, and Clary sage oil.

Another embodiment comprises cyclosporin A, Triton X-405 (70%)®, and Clary sage oil.

Another embodiment comprises cyclosporin A, Triton X-45®, and Clary sage oil.

Another embodiment comprises cyclosporin A, Triton X-705 (70%)®, and Clary sage oil.

Another embodiment comprises cyclosporin A, Volpo S-10®, and Clary sage oil.

Another embodiment comprises cyclosporin A, Volpo S-2®, and Clary sage oil.

Another embodiment comprises cyclosporin A, Volpo S-20®, and Clary sage oil.

Another embodiment comprises cyclosporin A, Volpo-10®, and Clary sage oil.

Another embodiment comprises cyclosporin A, Volpo-20®, and Clary sage oil.

Another embodiment comprises cyclosporin A, Volpo-3®, and Clary sage oil.

Another embodiment comprises cyclosporin A, Volpo-5®, and Clary sage oil.

Another embodiment comprises cyclosporin A, Abex 2515®, and Nutmeg oil.

Another embodiment comprises cyclosporin A, Abex 2525/40®, and Nutmeg oil.

Another embodiment comprises cyclosporin A, Abex 2535®, and Nutmeg oil.

Another embodiment comprises cyclosporin A, Abex 2545®, and Nutmeg oil.

Another embodiment comprises cyclosporin A, Acconon E®, and Nutmeg oil.

Another embodiment comprises cyclosporin A, Alfonic 1216CO-9®, and Nutmeg oil.

Another embodiment comprises cyclosporin A, Alfonic 1412-3®, and Nutmeg oil.

Another embodiment comprises cyclosporin A, Alfonic TDA-12®, and Nutmeg oil.

Another embodiment comprises cyclosporin A, Alfonic TDA-3®, and Nutmeg oil.

Another embodiment comprises cyclosporin A, Alfonic TDA-4®, and Nutmeg oil.

Another embodiment comprises cyclosporin A, Alfonic TDA-6®, and Nutmeg oil.

Another embodiment comprises cyclosporin A, Alfonic TDA-7®, and Nutmeg oil.

Another embodiment comprises cyclosporin A, Alfonic TDA-8®, and Nutmeg oil.

Another embodiment comprises cyclosporin A, Alfonic TDA-9®, and Nutmeg oil.

Another embodiment comprises cyclosporin A, Alkamuls 400-DO®, and Nutmeg oil.

Another embodiment comprises cyclosporin A, Alkamuls 400-MO/E®, and Nutmeg oil.

Another embodiment comprises cyclosporin A, Ameroxol OE-10®, and Nutmeg oil.

Another embodiment comprises cyclosporin A, Ameroxol OE-2®, and Nutmeg oil.

Another embodiment comprises cyclosporin A, Ameroxol OE-20®, and Nutmeg oil.

Another embodiment comprises cyclosporin A, Armix 176®, and Nutmeg oil.

Another embodiment comprises cyclosporin A, Armix 180-C®, and Nutmeg oil.

Another embodiment comprises cyclosporin A, Armix 183®, and Nutmeg oil.

Another embodiment comprises cyclosporin A, Brij 35®, and Nutmeg oil.

Another embodiment comprises cyclosporin A, Brij 35 Liquid®, and Nutmeg oil.

Another embodiment comprises cyclosporin A, Brij 35 SP®, and Nutmeg oil.

Another embodiment comprises cyclosporin A, Canasol MJ 2109®, and Nutmeg oil.

Another embodiment comprises cyclosporin A, Canasol MJ 52®, and Nutmeg oil.

Another embodiment comprises cyclosporin A, Canasol R 3600®, and Nutmeg oil.

Another embodiment comprises cyclosporin A, Canasol R 3603®, and Nutmeg oil.

Another embodiment comprises cyclosporin A, Canasol R 4000 H®, and Nutmeg oil.

Another embodiment comprises cyclosporin A, Cedepal CA-210®, and Nutmeg oil.

Another embodiment comprises cyclosporin A, Cedepal CA-520®, and Nutmeg oil.

Another embodiment comprises cyclosporin A, Cedepal CA-630®, and Nutmeg oil.

Another embodiment comprises cyclosporin A, Cedepal CA-720®, and Nutmeg oil.

Another embodiment comprises cyclosporin A, Cedepal CA-890®, and Nutmeg oil.

Another embodiment comprises cyclosporin A, Cedepal CA-897®, and Nutmeg oil.

Another embodiment comprises cyclosporin A, Cedepal CO-210®, and Nutmeg oil.

Another embodiment comprises cyclosporin A, Cedepal CO-430®, and Nutmeg oil.

Another embodiment comprises cyclosporin A, Cedepal CO-530®, and Nutmeg oil.

Another embodiment comprises cyclosporin A, Cedepal CO-630®, and Nutmeg oil.

Another embodiment comprises cyclosporin A, Cedepal CO-710®, and Nutmeg oil.

Another embodiment comprises cyclosporin A, Cedepal CO-730®, and Nutmeg oil.

Another embodiment comprises cyclosporin A, Cedepal CO-880®, and Nutmeg oil.

Another embodiment comprises cyclosporin A, Cedepal CO-887®, and Nutmeg oil.

Another embodiment comprises cyclosporin A, Cedepal CO-890®, and Nutmeg oil.

Another embodiment comprises cyclosporin A, Cedepal CO-897®, and Nutmeg oil.

Another embodiment comprises cyclosporin A, Cedepal CO-970®, and Nutmeg oil.

Another embodiment comprises cyclosporin A, Cedepal CO-977®, and Nutmeg oil.

Another embodiment comprises cyclosporin A, Cedepal CO-990®, and Nutmeg oil.

Another embodiment comprises cyclosporin A, Cedepal CO-997®, and Nutmeg oil.

Another embodiment comprises cyclosporin A, Chemal BP 261®, and Nutmeg oil.

Another embodiment comprises cyclosporin A, Chemal BP 262®, and Nutmeg oil.

Another embodiment comprises cyclosporin A, Chemal BP 3172®, and Nutmeg oil.

Another embodiment comprises cyclosporin A, Chemal BP 3174®, and Nutmeg oil.

Another embodiment comprises cyclosporin A, Chemal DA-4®, and Nutmeg oil.

Another embodiment comprises cyclosporin A, Chemal DA-6®, and Nutmeg oil.

Another embodiment comprises cyclosporin A, Chemal DA-9®, and Nutmeg oil.

Another embodiment comprises cyclosporin A, Chemal LA-23®, and Nutmeg oil.

Another embodiment comprises cyclosporin A, Chemal LA-4®, and Nutmeg oil.

Another embodiment comprises cyclosporin A, Chemal LA-9®, and Nutmeg oil.

Another embodiment comprises cyclosporin A, Chemal OA-20G®, and Nutmeg oil.

Another embodiment comprises cyclosporin A, Chemal OA-5®, and Nutmeg oil.

Another embodiment comprises cyclosporin A, Chemal OA-9®, and Nutmeg oil.

Another embodiment comprises cyclosporin A, Chemal TDA-12®, and Nutmeg oil.

Another embodiment comprises cyclosporin A, Chemal TDA-15®, and Nutmeg oil.

Another embodiment comprises cyclosporin A, Chemal TDA-3®, and Nutmeg oil.

Another embodiment comprises cyclosporin A, Chemal TDA-6®, and Nutmeg oil.

Another embodiment comprises cyclosporin A, Chemal TDA-9®, and Nutmeg oil.

Another embodiment comprises cyclosporin A, Chemax CO-16®, and Nutmeg oil.

Another embodiment comprises cyclosporin A, Chemax CO-200/50®, and Nutmeg oil.

Another embodiment comprises cyclosporin A, Chemax CO-25®, and Nutmeg oil.

Another embodiment comprises cyclosporin A, Chemax CO-30®, and Nutmeg oil.

Another embodiment comprises cyclosporin A, Chemax CO-36®, and Nutmeg oil.

Another embodiment comprises cyclosporin A, Chemax CO-40®, and Nutmeg oil.

Another embodiment comprises cyclosporin A, Chemax CO-5®, and Nutmeg oil.

Another embodiment comprises cyclosporin A, Chemax CO-80®, and Nutmeg oil.

Another embodiment comprises cyclosporin A, Chemax DNP-150/50®, and Nutmeg oil.

Another embodiment comprises cyclosporin A, Chemax DNP-18®, and Nutmeg oil.

Another embodiment comprises cyclosporin A, Chemax DNP-8®, and Nutmeg oil.

Another embodiment comprises cyclosporin A, Chemax E 200 ML®, and Nutmeg oil.

Another embodiment comprises cyclosporin A, Chemax E 200 MO®, and Nutmeg oil.

Another embodiment comprises cyclosporin A, Chemax E 400 ML®, and Nutmeg oil.

Another embodiment comprises cyclosporin A, Chemax E 400 MO®, and Nutmeg oil.

Another embodiment comprises cyclosporin A, Chemax E 600 ML®, and Nutmeg oil.

Another embodiment comprises cyclosporin A, Chemax E 600 MO®, and Nutmeg oil.

Another embodiment comprises cyclosporin A, Chemax E 1000 MO®, and Nutmeg oil.

Another embodiment comprises cyclosporin A, Chemax E 1000 MS®, and Nutmeg oil.

Another embodiment comprises cyclosporin A, Chemax E200 MS®, and Nutmeg oil.

Another embodiment comprises cyclosporin A, Chemax E400 MS®, and Nutmeg oil.

Another embodiment comprises cyclosporin A, Chemax E600 MS®, and Nutmeg oil.

Another embodiment comprises cyclosporin A, Chemax HCO-16®, and Nutmeg oil.

Another embodiment comprises cyclosporin A, Chemax HCO-200/50®, and Nutmeg oil.

Another embodiment comprises cyclosporin A, Chemax HCO-25®, and Nutmeg oil.

Another embodiment comprises cyclosporin A, Chemax HCO-5®, and Nutmeg oil.

Another embodiment comprises cyclosporin A, Chemax TO-16®, and Nutmeg oil.

Another embodiment comprises cyclosporin A, Chemax TO-8®, and Nutmeg oil.

Another embodiment comprises cyclosporin A, Colalube 3409®, and Nutmeg oil.

Another embodiment comprises cyclosporin A, Colalube 3411®, and Nutmeg oil.

Another embodiment comprises cyclosporin A, Colalube 3413®, and Nutmeg oil.

Another embodiment comprises cyclosporin A, Colalube 3414®, and Nutmeg oil.

Another embodiment comprises cyclosporin A, Colalube 3415®, and Nutmeg oil.

Another embodiment comprises cyclosporin A, Colalube 3416®, and Nutmeg oil.

Another embodiment comprises cyclosporin A, Colalube 3417®, and Nutmeg oil.

Another embodiment comprises cyclosporin A, Delonic NPE-100®, and Nutmeg oil.

Another embodiment comprises cyclosporin A, Delonic NPE-30®, and Nutmeg oil.

Another embodiment comprises cyclosporin A, Delonic NPE-40®, and Nutmeg oil.

Another embodiment comprises cyclosporin A, Delonic OPE-10®, and Nutmeg oil.

Another embodiment comprises cyclosporin A, Delonic OPE-12®, and Nutmeg oil.

Another embodiment comprises cyclosporin A, Delonic OPE-30®, and Nutmeg oil.

Another embodiment comprises cyclosporin A, Delonic OPE-40®, and Nutmeg oil.

Another embodiment comprises cyclosporin A, Delonic OPE-5®, and Nutmeg oil.

Another embodiment comprises cyclosporin A, Delonic OPE-7.5®, and Nutmeg oil.

Another embodiment comprises cyclosporin A, DePeg 16-CO®, and Nutmeg oil.

Another embodiment comprises cyclosporin A, DePeg 25-CO®, and Nutmeg oil.

Another embodiment comprises cyclosporin A, DePeg 30-CO®, and Nutmeg oil.

Another embodiment comprises cyclosporin A, DePeg 40-CO®, and Nutmeg oil.

Another embodiment comprises cyclosporin A, DePeg 5-CO®, and Nutmeg oil.

Another embodiment comprises cyclosporin A, DePeg 80-CO®, and Nutmeg oil.

Another embodiment comprises cyclosporin A, DeSonic S-100®, and Nutmeg oil.

Another embodiment comprises cyclosporin A, DeSonic S-114®, and Nutmeg oil.

Another embodiment comprises cyclosporin A, DeSonic S-405®, and Nutmeg oil.

Another embodiment comprises cyclosporin A, DeSonic S-45®, and Nutmeg oil.

Another embodiment comprises cyclosporin A, DeSonic TDA-9®, and Nutmeg oil.

Another embodiment comprises cyclosporin A, DeThox Acid O-14®, and Nutmeg oil.

Another embodiment comprises cyclosporin A, DeThox Acid O-16®, and Nutmeg oil.

Another embodiment comprises cyclosporin A, DeThox Acid O-9®, and Nutmeg oil.

Another embodiment comprises cyclosporin A, DeThox Acid TO-14®, and Nutmeg oil.

Another embodiment comprises cyclosporin A, DeThox Acid TO-16.5®, and Nutmeg oil.

Another embodiment comprises cyclosporin A, DeThox Acid TO-8.5®, and Nutmeg oil.

Another embodiment comprises cyclosporin A, Dur-Em 114®, and Nutmeg oil.

Another embodiment comprises cyclosporin A, Dur-Em 117®, and Nutmeg oil.

Another embodiment comprises cyclosporin A, Dur-Em 204®, and Nutmeg oil.

Another embodiment comprises cyclosporin A, Dur-Em 207®, and Nutmeg oil.

Another embodiment comprises cyclosporin A, Dur-Em 207E®, and Nutmeg oil.

Another embodiment comprises cyclosporin A, Emerest 2704®, and Nutmeg oil.

Another embodiment comprises cyclosporin A, Emerest 2712®, and Nutmeg oil.

Another embodiment comprises cyclosporin A, Emerest 2715®, and Nutmeg oil.

Another embodiment comprises cyclosporin A, Ethal EH-2®, and Nutmeg oil.

Another embodiment comprises cyclosporin A, Ethal EH-5®, and Nutmeg oil.

Another embodiment comprises cyclosporin A, Ethal OA-23®, and Nutmeg oil.

Another embodiment comprises cyclosporin A, Ethosperse CA-20®, and Nutmeg oil.

Another embodiment comprises cyclosporin A, Ethosperse LA-12®, and Nutmeg oil.

Another embodiment comprises cyclosporin A, Ethosperse LA-23®, and Nutmeg oil.

Another embodiment comprises cyclosporin A, Ethosperse LA-4®, and Nutmeg oil.

Another embodiment comprises cyclosporin A, Ethox 3095®, and Nutmeg oil.

Another embodiment comprises cyclosporin A, Ethox CO-16®, and Nutmeg oil.

Another embodiment comprises cyclosporin A, Ethox CO-200®, and Nutmeg oil.

Another embodiment comprises cyclosporin A, Ethox CO-25®, and Nutmeg oil.

Another embodiment comprises cyclosporin A, Ethox CO-30®, and Nutmeg oil.

Another embodiment comprises cyclosporin A, Ethox CO-36®, and Nutmeg oil.

Another embodiment comprises cyclosporin A, Ethox CO-40®, and Nutmeg oil.

Another embodiment comprises cyclosporin A, Ethox CO-5®, and Nutmeg oil.

Another embodiment comprises cyclosporin A, Ethox DO-14®, and Nutmeg oil.

Another embodiment comprises cyclosporin A, Ethox DO-9®, and Nutmeg oil.

Another embodiment comprises cyclosporin A, Ethox MA-8®, and Nutmeg oil.

Another embodiment comprises cyclosporin A, Ethox MI-14®, and Nutmeg oil.

Another embodiment comprises cyclosporin A, Ethox ML-14®, and Nutmeg oil.

Another embodiment comprises cyclosporin A, Ethox ML-5®, and Nutmeg oil.

Another embodiment comprises cyclosporin A, Ethox MO-14®, and Nutmeg oil.

Another embodiment comprises cyclosporin A, Ethox MO-9®, and Nutmeg oil.

Another embodiment comprises cyclosporin A, Ethox MS-14®, and Nutmeg oil.

Another embodiment comprises cyclosporin A, Ethox MS-23®, and Nutmeg oil.

Another embodiment comprises cyclosporin A, Ethox MS-40®, and Nutmeg oil.

Another embodiment comprises cyclosporin A, Ethox MS-8®, and Nutmeg oil.

Another embodiment comprises cyclosporin A, Ethox TO-16®, and Nutmeg oil.

Another embodiment comprises cyclosporin A, Ethox TO-8®, and Nutmeg oil.

Another embodiment comprises cyclosporin A, Ethylan SN-120®, and Nutmeg oil.

Another embodiment comprises cyclosporin A, Ethylan SN-70®, and Nutmeg oil.

Another embodiment comprises cyclosporin A, Ethylan SN-90®, and Nutmeg oil.

Another embodiment comprises cyclosporin A, Flexricin 13®, and Nutmeg oil.

Another embodiment comprises cyclosporin A, Flexricin 15®, and Nutmeg oil.

Another embodiment comprises cyclosporin A, Genapol 1261®, and Nutmeg oil.

Another embodiment comprises cyclosporin A, Generol 122 E25®, and Nutmeg oil.

Another embodiment comprises cyclosporin A, Glicepol 160®, and Nutmeg oil.

Another embodiment comprises cyclosporin A, Glicepol 560®, and Nutmeg oil.

Another embodiment comprises cyclosporin A, Glicepol GMS 20®, and Nutmeg oil.

Another embodiment comprises cyclosporin A, Hetoxide BN-13®, and Nutmeg oil.

Another embodiment comprises cyclosporin A, Hetoxide DNP-4®, and Nutmeg oil.

Another embodiment comprises cyclosporin A, Hetoxide DNP-9.6®, and Nutmeg oil.

Another embodiment comprises cyclosporin A, Hetoxide G-26®, and Nutmeg oil.

Another embodiment comprises cyclosporin A, Hetoxide G-7®, and Nutmeg oil.

Another embodiment comprises cyclosporin A, Hetoxide GT-20®, and Nutmeg oil.

Another embodiment comprises cyclosporin A, Hetoxide HC-40®, and Nutmeg oil.

Another embodiment comprises cyclosporin A, Hetoxol C-24®, and Nutmeg oil.

Another embodiment comprises cyclosporin A, Hetoxol CA-10®, and Nutmeg oil.

Another embodiment comprises cyclosporin A, Hetoxol CA-2®, and Nutmeg oil.

Another embodiment comprises cyclosporin A, Hetoxol CA-20®, and Nutmeg oil.

Another embodiment comprises cyclosporin A, Hetoxol CAWS®, and Nutmeg oil.

Another embodiment comprises cyclosporin A, Hetoxol CD-3®, and Nutmeg oil.

Another embodiment comprises cyclosporin A, Hetoxol CD-4®, and Nutmeg oil.

Another embodiment comprises cyclosporin A, Hetoxol CS-15®, and Nutmeg oil.

Another embodiment comprises cyclosporin A, Hetoxol CS-20®, and Nutmeg oil.

Another embodiment comprises cyclosporin A, Hetoxol CS-20D®, and Nutmeg oil.

Another embodiment comprises cyclosporin A, Hetoxol CS-25®, and Nutmeg oil.

Another embodiment comprises cyclosporin A, Hetoxol CS-50®, and Nutmeg oil.

Another embodiment comprises cyclosporin A, Hetoxol CS-9®, and Nutmeg oil.

Another embodiment comprises cyclosporin A, Hetoxol CSA-15®, and Nutmeg oil.

Another embodiment comprises cyclosporin A, Hetoxol D®, and Nutmeg oil.

Another embodiment comprises cyclosporin A, Hetoxol G®, and Nutmeg oil.

Another embodiment comprises cyclosporin A, Hetoxol J®, and Nutmeg oil.

Another embodiment comprises cyclosporin A, Hetoxol L®, and Nutmeg oil.

Another embodiment comprises cyclosporin A, Hetoxol L-23®, and Nutmeg oil.

Another embodiment comprises cyclosporin A, Hetoxol L-4®, and Nutmeg oil.

Another embodiment comprises cyclosporin A, Hetoxol L-9®, and Nutmeg oil.

Another embodiment comprises cyclosporin A, Hetoxol LS-9®, and Nutmeg oil.

Another embodiment comprises cyclosporin A, Hetoxol M-3®, and Nutmeg oil.

Another embodiment comprises cyclosporin A, Hetoxol OA-10 Special®, and Nutmeg oil.

Another embodiment comprises cyclosporin A, Hetoxol OA-20 Special®, and Nutmeg oil.

Another embodiment comprises cyclosporin A, Hetoxol OA-3 Special®, and Nutmeg oil.

Another embodiment comprises cyclosporin A, Hetoxol OA-5 Special®, and Nutmeg oil.

Another embodiment comprises cyclosporin A, Hetoxol OL-10®, and Nutmeg oil.

Another embodiment comprises cyclosporin A, Hetoxol OL-2®, and Nutmeg oil.

Another embodiment comprises cyclosporin A, Hetoxol OL-23®, and Nutmeg oil.

Another embodiment comprises cyclosporin A, Hetoxol OL-4®, and Nutmeg oil.

Another embodiment comprises cyclosporin A, Hetoxol OL-40®, and Nutmeg oil.

Another embodiment comprises cyclosporin A, Hetoxol OL-5®, and Nutmeg oil.

Another embodiment comprises cyclosporin A, Hetoxol PLA®, and Nutmeg oil.

Another embodiment comprises cyclosporin A, Hetoxol SP-15®, and Nutmeg oil.

Another embodiment comprises cyclosporin A, Hetoxol STA-10®, and Nutmeg oil.

Another embodiment comprises cyclosporin A, Hetoxol STA-2®, and Nutmeg oil.

Another embodiment comprises cyclosporin A, Hetoxol STA-30®, and Nutmeg oil.

Another embodiment comprises cyclosporin A, Hetoxol TD-12®, and Nutmeg oil.

Another embodiment comprises cyclosporin A, Hetoxol TD-18®, and Nutmeg oil.

Another embodiment comprises cyclosporin A, Hetoxol TD-3®, and Nutmeg oil.

Another embodiment comprises cyclosporin A, Hetoxol TD-6®, and Nutmeg oil.

Another embodiment comprises cyclosporin A, Hetoxol TD-9®, and Nutmeg oil.

Another embodiment comprises cyclosporin A, Hetoxol TDEP-15®, and Nutmeg oil.

Another embodiment comprises cyclosporin A, Hetoxol TDEP-63®, and Nutmeg oil.

Another embodiment comprises cyclosporin A, Iconol 24-12®, and Nutmeg oil.

Another embodiment comprises cyclosporin A, Iconol 24-23®, and Nutmeg oil.

Another embodiment comprises cyclosporin A, Iconol 24-4®, and Nutmeg oil.

Another embodiment comprises cyclosporin A, Iconol DA-4®, and Nutmeg oil.

Another embodiment comprises cyclosporin A, Iconol DA-6®, and Nutmeg oil.

Another embodiment comprises cyclosporin A, Iconol TDA-10®, and Nutmeg oil.

Another embodiment comprises cyclosporin A, Iconol TDA-3®, and Nutmeg oil.

Another embodiment comprises cyclosporin A, Iconol TDA-6®, and Nutmeg oil.

Another embodiment comprises cyclosporin A, Iconol TDA-8-90%®, and Nutmeg oil.

Another embodiment comprises cyclosporin A, Iconol TDA-9®, and Nutmeg oil.

Another embodiment comprises cyclosporin A, Igepal CA-210®, and Nutmeg oil.

Another embodiment comprises cyclosporin A, Igepal CA-407®, and Nutmeg oil.

Another embodiment comprises cyclosporin A, Igepal CA-420®, and Nutmeg oil.

Another embodiment comprises cyclosporin A, Igepal CA-520®, and Nutmeg oil.

Another embodiment comprises cyclosporin A, Igepal CA-620®, and Nutmeg oil.

Another embodiment comprises cyclosporin A, Igepal CA-630®, and Nutmeg oil.

Another embodiment comprises cyclosporin A, Igepal CA-720®, and Nutmeg oil.

Another embodiment comprises cyclosporin A, Igepal CA-877®, and Nutmeg oil.

Another embodiment comprises cyclosporin A, Igepal CA-887®, and Nutmeg oil.

Another embodiment comprises cyclosporin A, Igepal CA-897®, and Nutmeg oil.

Another embodiment comprises cyclosporin A, Igepal CO-210®, and Nutmeg oil.

Another embodiment comprises cyclosporin A, lgepal CO-430®, and Nutmeg oil.

Another embodiment comprises cyclosporin A, lgepal CO-520®, and Nutmeg oil.

Another embodiment comprises cyclosporin A, lgepal CO-530®, and Nutmeg oil.

Another embodiment comprises cyclosporin A, lgepal CO-610®, and Nutmeg oil.

Another embodiment comprises cyclosporin A, Igepal CO-630®, and Nutmeg oil.

Another embodiment comprises cyclosporin A, Igepal CO-630 Special®, and Nutmeg oil.

Another embodiment comprises cyclosporin A, Igepal CO-660®, and Nutmeg oil.

Another embodiment comprises cyclosporin A, Igepal CO-710®, and Nutmeg oil.

Another embodiment comprises cyclosporin A, Igepal CO-720®, and Nutmeg oil.

Another embodiment comprises cyclosporin A, lgepal CO-730®, and Nutmeg oil.

Another embodiment comprises cyclosporin A, lgepal CO-738®, and Nutmeg oil.

Another embodiment comprises cyclosporin A, Igepal CO-850®, and Nutmeg oil.

Another embodiment comprises cyclosporin A, lgepal CO-880®, and Nutmeg oil.

Another embodiment comprises cyclosporin A, Igepal CO-887®, and Nutmeg oil.

Another embodiment comprises cyclosporin A, lgepal CO-890®, and Nutmeg oil.

Another embodiment comprises cyclosporin A, Igepal CO-897®, and Nutmeg oil.

Another embodiment comprises cyclosporin A, Igepal CO-970®, and Nutmeg oil.

Another embodiment comprises cyclosporin A, Igepal CO-977®, and Nutmeg oil.

Another embodiment comprises cyclosporin A, Igepal CO-987®, and Nutmeg oil.

Another embodiment comprises cyclosporin A, Igepal CO-990 FLK®, and Nutmeg oil.

Another embodiment comprises cyclosporin A, lgepal CO-997®, and Nutmeg oil.

Another embodiment comprises cyclosporin A, Igepal DM-430®, and Nutmeg oil.

Another embodiment comprises cyclosporin A, lgepal DM-530®, and Nutmeg oil.

Another embodiment comprises cyclosporin A, lgepal DM-710®, and Nutmeg oil.

Another embodiment comprises cyclosporin A, lgepal DM-730®, and Nutmeg oil.

Another embodiment comprises cyclosporin A, Igepal DM-880®, and Nutmeg oil.

Another embodiment comprises cyclosporin A, Igepal DM-970 FLK®, and Nutmeg oil.

Another embodiment comprises cyclosporin A, lgepal OD-410®, and Nutmeg oil.

Another embodiment comprises cyclosporin A, Igepal RC-520®, and Nutmeg oil.

Another embodiment comprises cyclosporin A, Igepal RC-620®, and Nutmeg oil.

Another embodiment comprises cyclosporin A, lgepal RC-630®, and Nutmeg oil.

Another embodiment comprises cyclosporin A, Lexemul CS-20®, and Nutmeg oil.

Another embodiment comprises cyclosporin A, Lipo GMS 450®, and Nutmeg oil.

Another embodiment comprises cyclosporin A, Lipo Polyglycol 1000®, and Nutmeg oil.

Another embodiment comprises cyclosporin A, Lipo Polyglycol 200®, and Nutmeg oil.

Another embodiment comprises cyclosporin A, Lipo Polyglycol 300®, and Nutmeg oil.

Another embodiment comprises cyclosporin A, Lipo Polyglycol 3350®, and Nutmeg oil.

Another embodiment comprises cyclosporin A, Lipo Polyglycol 400®, and Nutmeg oil.

Another embodiment comprises cyclosporin A, Lipo Polyglycol 600®, and Nutmeg oil.

Another embodiment comprises cyclosporin A, Lipocol C-10®, and Nutmeg oil.

Another embodiment comprises cyclosporin A, Lipocol C-2®, and Nutmeg oil.

Another embodiment comprises cyclosporin A, Lipocol C-20®, and Nutmeg oil.

Another embodiment comprises cyclosporin A, Lipocol L-12®, and Nutmeg oil.

Another embodiment comprises cyclosporin A, Lipocol L-23®, and Nutmeg oil.

Another embodiment comprises cyclosporin A, Lipocol L-4®, and Nutmeg oil.

Another embodiment comprises cyclosporin A, Lipocol O-10®, and Nutmeg oil.

Another embodiment comprises cyclosporin A, Lipocol O-2®, and Nutmeg oil.

Another embodiment comprises cyclosporin A, Lipocol O-20®, and Nutmeg oil.

Another embodiment comprises cyclosporin A, Lipocol O-5®, and Nutmeg oil.

Another embodiment comprises cyclosporin A, Lipocol S-10®, and Nutmeg oil.

Another embodiment comprises cyclosporin A, Lipocol S-2®, and Nutmeg oil.

Another embodiment comprises cyclosporin A, Lipocol S-20®, and Nutmeg oil.

Another embodiment comprises cyclosporin A, Lipocol SC-15®, and Nutmeg oil.

Another embodiment comprises cyclosporin A, Lipocol SC-20®, and Nutmeg oil.

Another embodiment comprises cyclosporin A, Lipocol SC-4®, and Nutmeg oil.

Another embodiment comprises cyclosporin A, Lipocol TD-12®, and Nutmeg oil.

Another embodiment comprises cyclosporin A, Lumulse 40-O K®, and Nutmeg oil.

Another embodiment comprises cyclosporin A, Lumulse 42-O K®, and Nutmeg oil.

Another embodiment comprises cyclosporin A, Lumulse GMT-40®, and Nutmeg oil.

Another embodiment comprises cyclosporin A, Lumulse PEG 1450®, and Nutmeg oil.

Another embodiment comprises cyclosporin A, Lumulse PEG 1450 NF®, and Nutmeg oil.

Another embodiment comprises cyclosporin A, Lumulse PEG 8000®, and Nutmeg oil.

Another embodiment comprises cyclosporin A, Lumulse PGO®, and Nutmeg oil.

Another embodiment comprises cyclosporin A, Lumulse POE (2) Oleyl Amine®, and Nutmeg oil.

Another embodiment comprises cyclosporin A, Magrabar PGO-104®, and Nutmeg oil.

Another embodiment comprises cyclosporin A, Mapeg 200 ML®, and Nutmeg oil.

Another embodiment comprises cyclosporin A, Mapeg 400 MO®, and Nutmeg oil.

Another embodiment comprises cyclosporin A, Mapeg 400 MOT®, and Nutmeg oil.

Another embodiment comprises cyclosporin A, Mapeg 400DO®, and Nutmeg oil.

Another embodiment comprises cyclosporin A, Mapeg 400DOT®, and Nutmeg oil.

Another embodiment comprises cyclosporin A, Mapeg 600DOT®, and Nutmeg oil.

Another embodiment comprises cyclosporin A, Marlox B 24/80®, and Nutmeg oil.

Another embodiment comprises cyclosporin A, Marlox FK 86®, and Nutmeg oil.

Another embodiment comprises cyclosporin A, Marlox MO 154®, and Nutmeg oil.

Another embodiment comprises cyclosporin A, Mazox CDA CG®, and Nutmeg oil.

Another embodiment comprises cyclosporin A, Monoglycerides Glyceryl Monestearate®, and Nutmeg oil.

Another embodiment comprises cyclosporin A, Myrj 45®, and Nutmeg oil.

Another embodiment comprises cyclosporin A, Myrj 52®, and Nutmeg oil.

Another embodiment comprises cyclosporin A, Myrj 52S®, and Nutmeg oil.

Another embodiment comprises cyclosporin A, Myrj 53®, and Nutmeg oil.

Another embodiment comprises cyclosporin A, Myrj 59®, and Nutmeg oil.

Another embodiment comprises cyclosporin A, Norfox NP-12®, and Nutmeg oil.

Another embodiment comprises cyclosporin A, Norfox NP-4®, and Nutmeg oil.

Another embodiment comprises cyclosporin A, Norfox NP-6®, and Nutmeg oil.

Another embodiment comprises cyclosporin A, Norfox NP-887®, and Nutmeg oil.

Another embodiment comprises cyclosporin A, Norfox NP-9®, and Nutmeg oil.

Another embodiment comprises cyclosporin A, Norfox NP-977®, and Nutmeg oil.

Another embodiment comprises cyclosporin A, Norfox OP-100®, and Nutmeg oil.

Another embodiment comprises cyclosporin A, Norfox OP-102®, and Nutmeg oil.

Another embodiment comprises cyclosporin A, Norfox OP-114®, and Nutmeg oil.

Another embodiment comprises cyclosporin A, Norfox OP-45®, and Nutmeg oil.

Another embodiment comprises cyclosporin A, Pegosperse 100-L®, and Nutmeg oil.

Another embodiment comprises cyclosporin A, Pegosperse 100-O®, and Nutmeg oil.

Another embodiment comprises cyclosporin A, Pegosperse 100-S®, and Nutmeg oil.

Another embodiment comprises cyclosporin A, Pegosperse 1500-MS®, and Nutmeg oil.

Another embodiment comprises cyclosporin A, Pegosperse 1750-MS®, and Nutmeg oil.

Another embodiment comprises cyclosporin A, Pegosperse 200 DL®, and Nutmeg oil.

Another embodiment comprises cyclosporin A, Pegosperse 200-ML®, and Nutmeg oil.

Another embodiment comprises cyclosporin A, Pegosperse 400-DL®, and Nutmeg oil.

Another embodiment comprises cyclosporin A, Pegosperse 400-DO®, and Nutmeg oil.

Another embodiment comprises cyclosporin A, Pegosperse 400-DS®, and Nutmeg oil.

Another embodiment comprises cyclosporin A, Pegosperse 400-ML®, and Nutmeg oil.

Another embodiment comprises cyclosporin A, Pegosperse 400-MO®, and Nutmeg oil.

Another embodiment comprises cyclosporin A, Pegosperse 400-MOT®, and Nutmeg oil.

Another embodiment comprises cyclosporin A, Pegosperse 400-MS®, and Nutmeg oil.

Another embodiment comprises cyclosporin A, Pegosperse 50-DS®, and Nutmeg oil.

Another embodiment comprises cyclosporin A, Pegosperse 50-MS®, and Nutmeg oil.

Another embodiment comprises cyclosporin A, Pegosperse 600-ML®, and Nutmeg oil.

Another embodiment comprises cyclosporin A, Pegosperse 600-MS®, and Nutmeg oil.

Another embodiment comprises cyclosporin A, Plurafac A-38®, and Nutmeg oil.

Another embodiment comprises cyclosporin A, Plurafac B-25-5®, and Nutmeg oil.

Another embodiment comprises cyclosporin A, Plurafac B-26®, and Nutmeg oil.

Another embodiment comprises cyclosporin A, Plurafac D-25®, and Nutmeg oil.

Another embodiment comprises cyclosporin A, Plurafac LF 131®, and Nutmeg oil.

Another embodiment comprises cyclosporin A, Plurafac LF 4030®, and Nutmeg oil.

Another embodiment comprises cyclosporin A, Plurafac LF 7000®, and Nutmeg oil.

Another embodiment comprises cyclosporin A, Plurafac RA-20®, and Nutmeg oil.

Another embodiment comprises cyclosporin A, Plurafac RA-30®, and Nutmeg oil.

Another embodiment comprises cyclosporin A, Plurafac RA-40®, and Nutmeg oil.

Another embodiment comprises cyclosporin A, Pluronic 10R5®, and Nutmeg oil.

Another embodiment comprises cyclosporin A, Pluronic 17R2®, and Nutmeg oil.

Another embodiment comprises cyclosporin A, Pluronic 17R4®, and Nutmeg oil.

Another embodiment comprises cyclosporin A, Pluronic 25R2®, and Nutmeg oil.

Another embodiment comprises cyclosporin A, Pluronic 25R4®, and Nutmeg oil.

Another embodiment comprises cyclosporin A, Pluronic 31R1®, and Nutmeg oil.

Another embodiment comprises cyclosporin A, Pluronic F108®, and Nutmeg oil.

Another embodiment comprises cyclosporin A, Pluronic F127®, and Nutmeg oil.

Another embodiment comprises cyclosporin A, Pluronic F38®, and Nutmeg oil.

Another embodiment comprises cyclosporin A, Pluronic F68®, and Nutmeg oil.

Another embodiment comprises cyclosporin A, Pluronic F68LF®, and Nutmeg oil.

Another embodiment comprises cyclosporin A, Pluronic F77®, and Nutmeg oil.

Another embodiment comprises cyclosporin A, Pluronic F87®, and Nutmeg oil.

Another embodiment comprises cyclosporin A, Pluronic F88®, and Nutmeg oil.

Another embodiment comprises cyclosporin A, Pluronic F98®, and Nutmeg oil.

Another embodiment comprises cyclosporin A, Pluronic L10®, and Nutmeg oil.

Another embodiment comprises cyclosporin A, Pluronic L101®, and Nutmeg oil.

Another embodiment comprises cyclosporin A, Pluronic L121®, and Nutmeg oil.

Another embodiment comprises cyclosporin A, Pluronic L31®, and Nutmeg oil.

Another embodiment comprises cyclosporin A, Pluronic L35®, and Nutmeg oil.

Another embodiment comprises cyclosporin A, Pluronic L43®, and Nutmeg oil.

Another embodiment comprises cyclosporin A, Pluronic L44®, and Nutmeg oil.

Another embodiment comprises cyclosporin A, Pluronic L61®, and Nutmeg oil.

Another embodiment comprises cyclosporin A, Pluronic L62®, and Nutmeg oil.

Another embodiment comprises cyclosporin A, Pluronic L62D®, and Nutmeg oil.

Another embodiment comprises cyclosporin A, Pluronic L62LF®, and Nutmeg oil.

Another embodiment comprises cyclosporin A, Pluronic L64®, and Nutmeg oil.

Another embodiment comprises cyclosporin A, Pluronic L81®, and Nutmeg oil.

Another embodiment comprises cyclosporin A, Pluronic L92®, and Nutmeg oil.

Another embodiment comprises cyclosporin A, Pluronic P103®, and Nutmeg oil.

Another embodiment comprises cyclosporin A, Pluronic P104®, and Nutmeg oil.

Another embodiment comprises cyclosporin A, Pluronic P105®, and Nutmeg oil.

Another embodiment comprises cyclosporin A, Pluronic P123®, and Nutmeg oil.

Another embodiment comprises cyclosporin A, Pluronic P65®, and Nutmeg oil.

Another embodiment comprises cyclosporin A, Pluronic P84®, and Nutmeg oil.

Another embodiment comprises cyclosporin A, Pluronic P85®, and Nutmeg oil.

Another embodiment comprises cyclosporin A, Polyaldo 10-2-P®, and Nutmeg oil.

Another embodiment comprises cyclosporin A, Polyaldo DGDO®, and Nutmeg oil.

Another embodiment comprises cyclosporin A, Polyaldo HGDS®, and Nutmeg oil.

Another embodiment comprises cyclosporin A, Polyaldo TGMS®, and Nutmeg oil.

Another embodiment comprises cyclosporin A, Protegin V®, and Nutmeg oil.

Another embodiment comprises cyclosporin A, Protegin W®, and Nutmeg oil.

Another embodiment comprises cyclosporin A, Protegin WX®, and Nutmeg oil.

Another embodiment comprises cyclosporin A, Protegin XV®, and Nutmeg oil.

Another embodiment comprises cyclosporin A, Rhodasurf BC-420®, and Nutmeg oil.

Another embodiment comprises cyclosporin A, Rhodasurf BC-610®, and Nutmeg oil.

Another embodiment comprises cyclosporin A, Rhodasurf BC-630®, and Nutmeg oil.

Another embodiment comprises cyclosporin A, Rhodasurf BC-720®, and Nutmeg oil.

Another embodiment comprises cyclosporin A, Rhodasurf BC-840®, and Nutmeg oil.

Another embodiment comprises cyclosporin A, Rhodasurf L 1®, and Nutmeg oil.

Another embodiment comprises cyclosporin A, Rhodasurf L 12®, and Nutmeg oil.

Another embodiment comprises cyclosporin A, Rhodasurf L 2®, and Nutmeg oil.

Another embodiment comprises cyclosporin A, Rhodasurf L 20®, and Nutmeg oil.

Another embodiment comprises cyclosporin A, Rhodasurf L 3®, and Nutmeg oil.

Another embodiment comprises cyclosporin A, Rhodasurf L 30®, and Nutmeg oil.

Another embodiment comprises cyclosporin A, Rhodasurf L 4®, and Nutmeg oil.

Another embodiment comprises cyclosporin A, Rhodasurf L 50®, and Nutmeg oil.

Another embodiment comprises cyclosporin A, Rhodasurf L 7/90®, and Nutmeg oil.

Another embodiment comprises cyclosporin A, Rhodasurf LA-3®, and Nutmeg oil.

Another embodiment comprises cyclosporin A, Rhodasurf LA-7®, and Nutmeg oil.

Another embodiment comprises cyclosporin A, Rhodasurf LAN-23®, and Nutmeg oil.

Another embodiment comprises cyclosporin A, Rhodasurf ON-870®, and Nutmeg oil.

Another embodiment comprises cyclosporin A, Rhodasurf ON-877®, and Nutmeg oil.

Another embodiment comprises cyclosporin A, Rhodasurf TDA-8/5®, and Nutmeg oil.

Another embodiment comprises cyclosporin A, Ritapro 100®, and Nutmeg oil.

Another embodiment comprises cyclosporin A, Ritapro 165®, and Nutmeg oil.

Another embodiment comprises cyclosporin A, Ritapro 200®, and Nutmeg-oil.

Another embodiment comprises cyclosporin A, Ritapro 300®, and Nutmeg oil.

Another embodiment comprises cyclosporin A, Ritoleth 10®, and Nutmeg oil.

Another embodiment comprises cyclosporin A, Ritoleth 2®, and Nutmeg oil.

Another embodiment comprises cyclosporin A, Ritoleth 20®, and Nutmeg oil.

Another embodiment comprises cyclosporin A, Ritoleth 5®, and Nutmeg oil.

Another embodiment comprises cyclosporin A, Santone 10-10-0®, and Nutmeg oil.

Another embodiment comprises cyclosporin A, Santone 3-1-S XTR®, and Nutmeg oil.

Another embodiment comprises cyclosporin A, Santone 3-1-SH®, and Nutmeg oil.

Another embodiment comprises cyclosporin A, Santone 8-1-O®, and Nutmeg oil.

Another embodiment comprises cyclosporin A, Solulan 16®, and Nutmeg oil.

Another embodiment comprises cyclosporin A, Solulan 75®, and Nutmeg oil.

Another embodiment comprises cyclosporin A, Solulan C-24®, and Nutmeg oil.

Another embodiment comprises cyclosporin A, Solulan L-575®, and Nutmeg oil.

Another embodiment comprises cyclosporin A, Standamox CAW®, and Nutmeg oil.

Another embodiment comprises cyclosporin A, Surfine AZI-A®, and Nutmeg oil.

Another embodiment comprises cyclosporin A, Surfonic CO-15®, and Nutmeg oil.

Another embodiment comprises cyclosporin A, Surfonic CO-25®, and Nutmeg oil.

Another embodiment comprises cyclosporin A, Surfonic CO-30®, and Nutmeg oil.

Another embodiment comprises cyclosporin A, Surfonic CO-36®, and Nutmeg oil.

Another embodiment comprises cyclosporin A, Surfonic CO-42®, and Nutmeg oil.

Another embodiment comprises cyclosporin A, Surfonic E400-MO®, and Nutmeg oil.

Another embodiment comprises cyclosporin A, Surfonic L12-3®, and Nutmeg oil.

Another embodiment comprises cyclosporin A, Surfonic L12-6®, and Nutmeg oil.

Another embodiment comprises cyclosporin A, Surfonic L12-8®, and Nutmeg oil.

Another embodiment comprises cyclosporin A, Surfonic L24-12®, and Nutmeg oil.

Another embodiment comprises cyclosporin A, Surfonic L24-2®, and Nutmeg oil.

Another embodiment comprises cyclosporin A, Surfonic L24-22®, and Nutmeg oil.

Another embodiment comprises cyclosporin A, Surfonic L24-3®, and Nutmeg oil.

Another embodiment comprises cyclosporin A, Surfonic L24-4®, and Nutmeg oil.

Another embodiment comprises cyclosporin A, Surfonic L24-5®, and Nutmeg oil.

Another embodiment comprises cyclosporin A, Surfonic L24-7®, and Nutmeg oil.

Another embodiment comprises cyclosporin A, Surfonic L24-7.1®, and Nutmeg oil.

Another embodiment comprises cyclosporin A, Surfonic L24-9®, and Nutmeg oil.

Another embodiment comprises cyclosporin A, Surfonic LF-17®, and Nutmeg oil.

Another embodiment comprises cyclosporin A, Surfonic LF-18®, and Nutmeg oil.

Another embodiment comprises cyclosporin A, Surfonic LF-37®, and Nutmeg oil.

Another embodiment comprises cyclosporin A, Surfonic LF-41®, and Nutmeg oil.

Another embodiment comprises cyclosporin A, Surfonic LF-42®, and Nutmeg oil.

Another embodiment comprises cyclosporin A, Surfonic LF-47®, and Nutmeg oil.

Another embodiment comprises cyclosporin A, Surfonic LF-68®, and Nutmeg oil.

Another embodiment comprises cyclosporin A, Surfonic N-10®, and Nutmeg oil.

Another embodiment comprises cyclosporin A, Surfonic N-100®, and Nutmeg oil.

Another embodiment comprises cyclosporin A, Surfonic N-102®, and Nutmeg oil.

Another embodiment comprises cyclosporin A, Surfonic N-120®, and Nutmeg oil.

Another embodiment comprises cyclosporin A, Surfonic N-150®, and Nutmeg oil.

Another embodiment comprises cyclosporin A, Surfonic N-200®, and Nutmeg oil.

Another embodiment comprises cyclosporin A, Surfonic N-300®, and Nutmeg oil.

Another embodiment comprises cyclosporin A, Surfonic N-31.5®, and Nutmeg oil.

Another embodiment comprises cyclosporin A, Surfonic N-40®, and Nutmeg oil.

Another embodiment comprises cyclosporin A, Surfonic N-400®, and Nutmeg oil.

Another embodiment comprises cyclosporin A, Surfonic N-60®, and Nutmeg oil.

Another embodiment comprises cyclosporin A, Surfonic N-85®, and Nutmeg oil.

Another embodiment comprises cyclosporin A, Surfonic N-95®, and Nutmeg oil.

Another embodiment comprises cyclosporin A, Surfonic NB-18®, and Nutmeg oil.

Another embodiment comprises cyclosporin A, Surfonic NB-23®, and Nutmeg oil.

Another embodiment comprises cyclosporin A, Surfonic NB-25®, and Nutmeg oil.

Another embodiment comprises cyclosporin A, Surfonic NB-307®, and Nutmeg oil.

Another embodiment comprises cyclosporin A, Surfonic NB-31®, and Nutmeg oil.

Another embodiment comprises cyclosporin A, Surfonic NB-41®, and Nutmeg oil.

Another embodiment comprises cyclosporin A, Surfonic NB-43®, and Nutmeg oil.

Another embodiment comprises cyclosporin A, Surfonic OP-100®, and Nutmeg oil.

Another embodiment comprises cyclosporin A, Surfonic OP-120®, and Nutmeg oil.

Another embodiment comprises cyclosporin A, Surfonic OP-15®, and Nutmeg oil.

Another embodiment comprises cyclosporin A, Surfonic OP-35®, and Nutmeg oil.

Another embodiment comprises cyclosporin A, Surfonic OP-70®, and Nutmeg oil.

Another embodiment comprises cyclosporin A, Surfonic P1®, and Nutmeg oil.

Another embodiment comprises cyclosporin A, Surfonic P3®, and Nutmeg oil.

Another embodiment comprises cyclosporin A, Surfonic P5®, and Nutmeg oil.

Another embodiment comprises cyclosporin A, Surfonic P6®, and Nutmeg oil.

Another embodiment comprises cyclosporin A, Surfonic POA-17R2®, and Nutmeg oil.

Another embodiment comprises cyclosporin A, Surfonic POA-25R2®, and Nutmeg oil.

Another embodiment comprises cyclosporin A, Surfonic TDA-11®, and Nutmeg oil.

Another embodiment comprises cyclosporin A, Surfonic TDA-3B®, and Nutmeg oil.

Another embodiment comprises cyclosporin A, Surfonic TDA-6®, and Nutmeg oil.

Another embodiment comprises cyclosporin A, Surfonic TDA-6/88®, and Nutmeg oil.

Another embodiment comprises cyclosporin A, Surfonic TDA-8®, and Nutmeg oil.

Another embodiment comprises cyclosporin A, Surfonic TDA-8/90®, and Nutmeg oil.

Another embodiment comprises cyclosporin A, Surfonic TDA-9®, and Nutmeg oil.

Another embodiment comprises cyclosporin A, Surfonic TX-CCN®, and Nutmeg oil.

Another embodiment comprises cyclosporin A, Tagat CH 40®, and Nutmeg oil.

Another embodiment comprises cyclosporin A, Tagat CH 60®, and Nutmeg oil.

Another embodiment comprises cyclosporin A, Tagat L 2®, and Nutmeg oil.

Another embodiment comprises cyclosporin A, Tagat S®, and Nutmeg oil.

Another embodiment comprises cyclosporin A, Tagat S 2®, and Nutmeg oil.

Another embodiment comprises cyclosporin A, Tally 100 Plus®, and Nutmeg oil.

Another embodiment comprises cyclosporin A, T-Det A106®, and Nutmeg oil.

Another embodiment comprises cyclosporin A, T-Det A109®, and Nutmeg oil.

Another embodiment comprises cyclosporin A, T-Det A136®, and Nutmeg oil.

Another embodiment comprises cyclosporin A, T-Det A139®, and Nutmeg oil.

Another embodiment comprises cyclosporin A, T-Det A2412®, and Nutmeg oil.

Another embodiment comprises cyclosporin A, T-Det A243®, and Nutmeg oil.

Another embodiment comprises cyclosporin A, T-Det A247®, and Nutmeg oil.

Another embodiment comprises cyclosporin A, T-Det A249®, and Nutmeg oil.

Another embodiment comprises cyclosporin A, T-Det A467®, and Nutmeg oil.

Another embodiment comprises cyclosporin A, T-Det COE®, and Nutmeg oil.

Another embodiment comprises cyclosporin A, T-Det DD-10®, and Nutmeg oil.

Another embodiment comprises cyclosporin A, T-Det DD-5®, and Nutmeg oil.

Another embodiment comprises cyclosporin A, T-Det DD-7®, and Nutmeg oil.

Another embodiment comprises cyclosporin A, T-Det EPO-104®, and Nutmeg oil.

Another embodiment comprises cyclosporin A, T-Det EPO-61®, and Nutmeg oil.

Another embodiment comprises cyclosporin A, T-Det EPO-62®, and Nutmeg oil.

Another embodiment comprises cyclosporin A, T-Det EPO-64®, and Nutmeg oil.

Another embodiment comprises cyclosporin A, T-Det N-1.5®, and Nutmeg oil.

Another embodiment comprises cyclosporin A, T-Det N-10.5®, and Nutmeg oil.

Another embodiment comprises cyclosporin A, T-Det N-100®, and Nutmeg oil.

Another embodiment comprises cyclosporin A, T-Det N-1007®, and Nutmeg oil.

Another embodiment comprises cyclosporin A, T-Det N-12®, and Nutmeg oil.

Another embodiment comprises cyclosporin A, T-Det N-14®, and Nutmeg oil.

Another embodiment comprises cyclosporin A, T-Det N-20®, and Nutmeg oil.

Another embodiment comprises cyclosporin A, T-Det N-30®, and Nutmeg oil.

Another embodiment comprises cyclosporin A, T-Det N-307®, and Nutmeg oil.

Another embodiment comprises cyclosporin A, T-Det N-4®, and Nutmeg oil.

Another embodiment comprises cyclosporin A, T-Det N-40®, and Nutmeg oil.

Another embodiment comprises cyclosporin A, T-Det N-407®, and Nutmeg oil.

Another embodiment comprises cyclosporin A, T-Det N-50®, and Nutmeg oil.

Another embodiment comprises cyclosporin A, T-Det N-507®, and Nutmeg oil.

Another embodiment comprises cyclosporin A, T-Det N-6®, and Nutmeg oil.

Another embodiment comprises cyclosporin A, T-Det N-70®, and Nutmeg oil.

Another embodiment comprises cyclosporin A, T-Det N-8®, and Nutmeg oil.

Another embodiment comprises cyclosporin A, T-Det N-9.5®, and Nutmeg oil.

Another embodiment comprises cyclosporin A, T-Det O-12®, and Nutmeg oil.

Another embodiment comprises cyclosporin A, T-Det O-165®, and Nutmeg oil.

Another embodiment comprises cyclosporin A, T-Det O-307®, and Nutmeg oil.

Another embodiment comprises cyclosporin A, T-Det O-40®, and Nutmeg oil.

Another embodiment comprises cyclosporin A, T-Det O-407®, and Nutmeg oil.

Another embodiment comprises cyclosporin A, T-Det O-6®, and Nutmeg oil.

Another embodiment comprises cyclosporin A, T-Det O-8®, and Nutmeg oil.

Another embodiment comprises cyclosporin A, T-Det O-9®, and Nutmeg oil.

Another embodiment comprises cyclosporin A, Tegin®, and Nutmeg oil.

Another embodiment comprises cyclosporin A, Tergitol 15-S-12®, and Nutmeg oil.

Another embodiment comprises cyclosporin A, Tergitol 15-S-15®, and Nutmeg oil.

Another embodiment comprises cyclosporin A, Tergitol 15-S-20®, and Nutmeg oil.

Another embodiment comprises cyclosporin A, Tergitol 15-S-20 (80AQ)®, and Nutmeg oil.

Another embodiment comprises cyclosporin A, Tergitol 15-S-3®, and Nutmeg oil.

Another embodiment comprises cyclosporin A, Tergitol 15-S-30®, and Nutmeg oil.

Another embodiment comprises cyclosporin A, Tergitol 15-S-40®, and Nutmeg oil.

Another embodiment comprises cyclosporin A, Tergitol 15-S-40 (70AQ)®, and Nutmeg oil.

Another embodiment comprises cyclosporin A, Tergitol 15-S-5®, and Nutmeg oil.

Another embodiment comprises cyclosporin A, Tergitol 15-S-7®, and Nutmeg oil.

Another embodiment comprises cyclosporin A, Tergitol 15-S-9®, and Nutmeg oil.

Another embodiment comprises cyclosporin A, Tergitol L-101®, and Nutmeg oil.

Another embodiment comprises cyclosporin A, Tergitol L-61®, and Nutmeg oil.

Another embodiment comprises cyclosporin A, Tergitol L-62®, and Nutmeg oil.

Another embodiment comprises cyclosporin A, Tergitol L-64®, and Nutmeg oil.

Another embodiment comprises cyclosporin A, Tergitol L-81®, and Nutmeg oil.

Another embodiment comprises cyclosporin A, Tergitol NP-10®, and Nutmeg oil.

Another embodiment comprises cyclosporin A, Tergitol NP-11®, and Nutmeg oil.

Another embodiment comprises cyclosporin A, Tergitol NP-12®, and Nutmeg oil.

Another embodiment comprises cyclosporin A, Tergitol NP-13®, and Nutmeg oil.

Another embodiment comprises cyclosporin A, Tergitol NP-15®, and Nutmeg oil.

Another embodiment comprises cyclosporin A, Tergitol NP-30®, and Nutmeg oil.

Another embodiment comprises cyclosporin A, Tergitol NP-30 (70 AQ)®, and Nutmeg oil.

Another embodiment comprises cyclosporin A, Tergitol NP-4®, and Nutmeg oil.

Another embodiment comprises cyclosporin A, Tergitol NP-40®, and Nutmeg oil.

Another embodiment comprises cyclosporin A, Tergitol NP-40 (70 AQ)®, and Nutmeg oil.

Another embodiment comprises cyclosporin A, Tergitol NP-50 (70 AQ)®, and Nutmeg oil.

Another embodiment comprises cyclosporin A, Tergitol NP-55 (70 AQ)®, and Nutmeg oil.

Another embodiment comprises cyclosporin A, Tergitol NP-6®, and Nutmeg oil.

Another embodiment comprises cyclosporin A, Tergitol NP-7®, and Nutmeg oil.

Another embodiment comprises cyclosporin A, Tergitol NP-70 (70 AQ)®, and Nutmeg oil.

Another embodiment comprises cyclosporin A, Tergitol NP-8®, and Nutmeg oil.

Another embodiment comprises cyclosporin A, Tergitol NP-9®, and Nutmeg oil.

Another embodiment comprises cyclosporin A, Tergitol NP-9.5®, and Nutmeg oil.

Another embodiment comprises cyclosporin A, Tergitol TMN-10®, and Nutmeg oil.

Another embodiment comprises cyclosporin A, Tergitol TMN-3®, and Nutmeg oil.

Another embodiment comprises cyclosporin A, Tergitol TMN-6®, and Nutmeg oil.

Another embodiment comprises cyclosporin A, Tergitol XD®, and Nutmeg oil.

Another embodiment comprises cyclosporin A, Tergitol XH®, and Nutmeg oil.

Another embodiment comprises cyclosporin A, Tergitol XJ®, and Nutmeg oil.

Another embodiment comprises cyclosporin A, Tetronic 1107®, and Nutmeg oil.

Another embodiment comprises cyclosporin A, Tetronic 1307®, and Nutmeg oil.

Another embodiment comprises cyclosporin A, Tetronic 150R1®, and Nutmeg oil.

Another embodiment comprises cyclosporin A, Tetronic 304®, and Nutmeg oil.

Another embodiment comprises cyclosporin A, Tetronic 701®, and Nutmeg oil.

Another embodiment comprises cyclosporin A, Tetronic 901®, and Nutmeg oil.

Another embodiment comprises cyclosporin A, Tetronic 904®, and Nutmeg oil.

Another embodiment comprises cyclosporin A, Tetronic 908®, and Nutmeg oil.

Another embodiment comprises cyclosporin A, Tetronic 90R4®, and Nutmeg oil.

Another embodiment comprises cyclosporin A, Tomadol 1-3®, and Nutmeg oil.

Another embodiment comprises cyclosporin A, Tomadol 1-5®, and Nutmeg oil.

Another embodiment comprises cyclosporin A, Tomadol 1-7®, and Nutmeg oil.

Another embodiment comprises cyclosporin A, Tomadol 23-1®, and Nutmeg oil.

Another embodiment comprises cyclosporin A, Tomadol 23-3®, and Nutmeg oil.

Another embodiment comprises cyclosporin A, Tomadol 23-5®, and Nutmeg oil.

Another embodiment comprises cyclosporin A, Tomadol 23-6.5®, and Nutmeg oil.

Another embodiment comprises cyclosporin A, Tomadol 25-12®, and Nutmeg oil.

Another embodiment comprises cyclosporin A, Tomadol 25-3®, and Nutmeg oil.

Another embodiment comprises cyclosporin A, Tomadol 25-7®, and Nutmeg oil.

Another embodiment comprises cyclosporin A, Tomadol 25-9®, and Nutmeg oil.

Another embodiment comprises cyclosporin A, Tomadol 45-13®, and Nutmeg oil.

Another embodiment comprises cyclosporin A, Tomadol 45-2.5®, and Nutmeg oil.

Another embodiment comprises cyclosporin A, Tomadol 45-7®, and Nutmeg oil.

Another embodiment comprises cyclosporin A, Tomadol 91-2.5®, and Nutmeg oil.

Another embodiment comprises cyclosporin A, Tomadol 91-6®, and Nutmeg oil.

Another embodiment comprises cyclosporin A, Tomadol 91-8®, and Nutmeg oil.

Another embodiment comprises cyclosporin A, Triton X-100®, and Nutmeg oil.

Another embodiment comprises cyclosporin A, Triton X-102®, and Nutmeg oil.

Another embodiment comprises cyclosporin A, Triton X-114®, and Nutmeg oil.

Another embodiment comprises cyclosporin A, Triton X-15®, and Nutmeg oil.

Another embodiment comprises cyclosporin A, Triton X-165 (70%)®, and Nutmeg oil.

Another embodiment comprises cyclosporin A, Triton X-207®, and Nutmeg oil.

Another embodiment comprises cyclosporin A, Triton X-305 (70%)®, and Nutmeg oil.

Another embodiment comprises cyclosporin A, Triton X-35®, and Nutmeg oil.

Another embodiment comprises cyclosporin A, Triton X-405 (70%)®, and Nutmeg oil.

Another embodiment comprises cyclosporin A, Triton X-45®, and Nutmeg oil.

Another embodiment comprises cyclosporin A, Triton X-705 (70%)®, and Nutmeg oil.

Another embodiment comprises cyclosporin A, Volpo S-10®, and Nutmeg oil.

Another embodiment comprises cyclosporin A, Volpo S-2®, and Nutmeg oil.

Another embodiment comprises cyclosporin A, Volpo S-20®, and Nutmeg oil.

Another embodiment comprises cyclosporin A, Volpo-10®, and Nutmeg oil.

Another embodiment comprises cyclosporin A, Volpo-20®, and Nutmeg oil.

Another embodiment comprises cyclosporin A, Volpo-3®, and Nutmeg oil.

Another embodiment comprises cyclosporin A, Volpo-5®, and Nutmeg oil.

Another embodiment comprises cyclosporin A, Abex 2515®, and Tea tree oil.

Another embodiment comprises cyclosporin A, Abex 2525/40®, and Tea tree oil.

Another embodiment comprises cyclosporin A, Abex 2535®, and Tea tree oil.

Another embodiment comprises cyclosporin A, Abex 2545®, and Tea tree oil.

Another embodiment comprises cyclosporin A, Acconon E®, and Tea tree oil.

Another embodiment comprises cyclosporin A, Alfonic 1216CO-9®, and Tea tree oil.

Another embodiment comprises cyclosporin A, Alfonic 1412-3®, and Tea tree oil.

Another embodiment comprises cyclosporin A, Alfonic TDA-12®, and Tea tree oil.

Another embodiment comprises cyclosporin A, Alfonic TDA-3®, and Tea tree oil.

Another embodiment comprises cyclosporin A, Alfonic TDA-4®, and Tea tree oil.

Another embodiment comprises cyclosporin A, Alfonic TDA-6®, and Tea tree oil.

Another embodiment comprises cyclosporin A, Alfonic TDA-7®, and Tea tree oil.

Another embodiment comprises cyclosporin A, Alfonic TDA-8®, and Tea tree oil.

Another embodiment comprises cyclosporin A, Alfonic TDA-9®, and Tea tree oil.

Another embodiment comprises cyclosporin A, Alkamuls 400-DO®, and Tea tree oil.

Another embodiment comprises cyclosporin A, Alkamuls 400-MO/E®, and Tea tree oil.

Another embodiment comprises cyclosporin A, Ameroxol OE-10®, and Tea tree oil.

Another embodiment comprises cyclosporin A, Ameroxol OE-2®, and Tea tree oil.

Another embodiment comprises cyclosporin A, Ameroxol OE-20®, and Tea tree oil.

Another embodiment comprises cyclosporin A, Armix 176®, and Tea tree oil.

Another embodiment comprises cyclosporin A, Armix 180-C®, and Tea tree oil.

Another embodiment comprises cyclosporin A, Armix 183®, and Tea tree oil.

Another embodiment comprises cyclosporin A, Brij 35®, and Tea tree oil.

Another embodiment comprises cyclosporin A, Brij 35 Liquid®, and Tea tree oil.

Another embodiment comprises cyclosporin A, Brij 35 SP®, and Tea tree oil.

Another embodiment comprises cyclosporin A, Canasol MJ 2109®, and Tea tree oil.

Another embodiment comprises cyclosporin A, Canasol MJ 52®, and Tea tree oil.

Another embodiment comprises cyclosporin A, Canasol R 3600®, and Tea tree oil.

Another embodiment comprises cyclosporin A, Canasol R 3603®, and Tea tree oil.

Another embodiment comprises cyclosporin A, Canasol R 4000 Ho, and Tea tree oil.

Another embodiment comprises cyclosporin A, Cedepal CA-210®, and Tea tree oil.

Another embodiment comprises cyclosporin A, Cedepal CA-520®, and Tea tree oil.

Another embodiment comprises cyclosporin A, Cedepal CA-630®, and Tea tree oil.

Another embodiment comprises cyclosporin A, Cedepal CA-720®, and Tea tree oil.

Another embodiment comprises cyclosporin A, Cedepal CA-890®, and Tea tree oil.

Another embodiment comprises cyclosporin A, Cedepal CA-897®, and Tea tree oil.

Another embodiment comprises cyclosporin A, Cedepal CO-210®, and Tea tree oil.

Another embodiment comprises cyclosporin A, Cedepal CO-430®, and Tea tree oil.

Another embodiment comprises cyclosporin A, Cedepal CO-530®, and Tea tree oil.

Another embodiment comprises cyclosporin A, Cedepal CO-630®, and Tea tree oil.

Another embodiment comprises cyclosporin A, Cedepal CO-710®, and Tea tree oil.

Another embodiment comprises cyclosporin A, Cedepal CO-730®, and Tea tree oil.

Another embodiment comprises cyclosporin A, Cedepal CO-880®, and Tea tree oil.

Another embodiment comprises cyclosporin A, Cedepal CO-887®, and Tea tree oil.

Another embodiment comprises cyclosporin A, Cedepal CO-890®, and Tea tree oil.

Another embodiment comprises cyclosporin A, Cedepal CO-897®, and Tea tree oil.

Another embodiment comprises cyclosporin A, Cedepal CO-970®, and Tea tree oil.

Another embodiment comprises cyclosporin A, Cedepal CO-977®, and Tea tree oil.

Another embodiment comprises cyclosporin A, Cedepal CO-990®, and Tea tree oil.

Another embodiment comprises cyclosporin A, Cedepal CO-997®, and Tea tree oil.

Another embodiment comprises cyclosporin A, Chemal BP 261®, and Tea tree oil.

Another embodiment comprises cyclosporin A, Chemal BP 262®, and Tea tree oil.

Another embodiment comprises cyclosporin A, Chemal BP 3172®, and Tea tree oil.

Another embodiment comprises cyclosporin A, Chemal BP 3174®, and Tea tree oil.

Another embodiment comprises cyclosporin A, Chemal DA-4®, and Tea tree oil.

Another embodiment comprises cyclosporin A, Chemal DA-6®, and Tea tree oil.

Another embodiment comprises cyclosporin A, Chemal DA-9®, and Tea tree oil.

Another embodiment comprises cyclosporin A, Chemal LA-23®, and Tea tree oil.

Another embodiment comprises cyclosporin A, Chemal LA-4®, and Tea tree oil.

Another embodiment comprises cyclosporin A, Chemal LA-9®, and Tea tree oil.

Another embodiment comprises cyclosporin A, Chemal OA-20G®, and Tea tree oil.

Another embodiment comprises cyclosporin A, Chemal OA-5®, and Tea tree oil.

Another embodiment comprises cyclosporin A, Chemal OA-9®, and Tea tree oil.

Another embodiment comprises cyclosporin A, Chemal TDA-12®, and Tea tree oil.

Another embodiment comprises cyclosporin A, Chemal TDA-15®, and Tea tree oil.

Another embodiment comprises cyclosporin A, Chemal TDA-3®, and Tea tree oil.

Another embodiment comprises cyclosporin A, Chemal TDA-6®, and Tea tree oil.

Another embodiment comprises cyclosporin A, Chemal TDA-9®, and Tea tree oil.

Another embodiment comprises cyclosporin A, Chemax CO-16®, and Tea tree oil.

Another embodiment comprises cyclosporin A, Chemax CO-200/50®, and Tea tree oil.

Another embodiment comprises cyclosporin A, Chemax CO-25®, and Tea tree oil.

Another embodiment comprises cyclosporin A, Chemax CO-30®, and Tea tree oil.

Another embodiment comprises cyclosporin A, Chemax CO-36®, and Tea tree oil.

Another embodiment comprises cyclosporin A, Chemax CO-40®, and Tea tree oil.

Another embodiment comprises cyclosporin A, Chemax CO-5®, and Tea tree oil.

Another embodiment comprises cyclosporin A, Chemax CO-80®, and Tea tree oil.

Another embodiment comprises cyclosporin A, Chemax DNP-150/50®, and Tea tree oil.

Another embodiment comprises cyclosporin A, Chemax DNP-18®, and Tea tree oil.

Another embodiment comprises cyclosporin A, Chemax DNP-8®, and Tea tree oil.

Another embodiment comprises cyclosporin A, Chemax E 200 ML®, and Tea tree oil.

Another embodiment comprises cyclosporin A, Chemax E 200 MO®, and Tea tree oil.

Another embodiment comprises cyclosporin A, Chemax E 400 ML®, and Tea tree oil.

Another embodiment comprises cyclosporin A, Chemax E 400 MO®, and Tea tree oil.

Another embodiment comprises cyclosporin A, Chemax E 600 ML®, and Tea tree oil.

Another embodiment comprises cyclosporin A, Chemax E 600 MO®, and Tea tree oil.

Another embodiment comprises cyclosporin A, Chemax E 1000 MO®, and Tea tree oil.

Another embodiment comprises cyclosporin A, Chemax E 1000 MS®, and Tea tree oil.

Another embodiment comprises cyclosporin A, Chemax E200 MS®, and Tea tree oil.

Another embodiment comprises cyclosporin A, Chemax E400 MS®, and Tea tree oil.

Another embodiment comprises cyclosporin A, Chemax E600 MS®, and Tea tree oil.

Another embodiment comprises cyclosporin A, Chemax HCO-16®, and Tea tree oil.

Another embodiment comprises cyclosporin A, Chemax HCO-200/50®, and Tea tree oil.

Another embodiment comprises cyclosporin A, Chemax HCO-25®, and Tea tree oil.

Another embodiment comprises cyclosporin A, Chemax HCO-5®, and Tea tree oil.

Another embodiment comprises cyclosporin A, Chemax TO-16®, and Tea tree oil.

Another embodiment comprises cyclosporin A, Chemax TO-8®, and Tea tree oil.

Another embodiment comprises cyclosporin A, Colalube 3409®, and Tea tree oil.

Another embodiment comprises cyclosporin A, Colalube 3411®, and Tea tree oil.

Another embodiment comprises cyclosporin A, Colalube 3413®, and Tea tree oil.

Another embodiment comprises cyclosporin A, Colalube 3414®, and Tea tree oil.

Another embodiment comprises cyclosporin A, Colalube 3415®, and Tea tree oil.

Another embodiment comprises cyclosporin A, Colalube 3416®, and Tea tree oil.

Another embodiment comprises cyclosporin A, Colalube 3417®, and Tea tree oil.

Another embodiment comprises cyclosporin A, Delonic NPE-100®, and Tea tree oil.

Another embodiment comprises cyclosporin A, Delonic NPE-30®, and Tea tree oil.

Another embodiment comprises cyclosporin A, Delonic NPE-40®, and Tea tree oil.

Another embodiment comprises cyclosporin A, Delonic OPE-10®, and Tea tree oil.

Another embodiment comprises cyclosporin A, Delonic OPE-12®, and Tea tree oil.

Another embodiment comprises cyclosporin A, Delonic OPE-30®, and Tea tree oil.

Another embodiment comprises cyclosporin A, Delonic OPE-40®, and Tea tree oil.

Another embodiment comprises cyclosporin A, Delonic OPE-5®, and Tea tree oil.

Another embodiment comprises cyclosporin A, Delonic OPE-7.5®, and Tea tree oil.

Another embodiment comprises cyclosporin A, DePeg 16-CO®, and Tea tree oil.

Another embodiment comprises cyclosporin A, DePeg 25-CO®, and Tea tree oil.

Another embodiment comprises cyclosporin A, DePeg 30-CO®, and Tea tree oil.

Another embodiment comprises cyclosporin A, DePeg 40-CO®, and Tea tree oil.

Another embodiment comprises cyclosporin A, DePeg 5-CO®, and Tea tree oil.

Another embodiment comprises cyclosporin A, DePeg 80-CO®, and Tea tree oil.

Another embodiment comprises cyclosporin A, DeSonic S-100®, and Tea tree oil.

Another embodiment comprises cyclosporin A, DeSonic S-114®, and Tea tree oil.

Another embodiment comprises cyclosporin A, DeSonic S-405®, and Tea tree oil.

Another embodiment comprises cyclosporin A, DeSonic S-45®, and Tea tree oil.

Another embodiment comprises cyclosporin A, DeSonic TDA-9®, and Tea tree oil.

Another embodiment comprises cyclosporin A, DeThox Acid O-14®, and Tea tree oil.

Another embodiment comprises cyclosporin A, DeThox Acid O-16®, and Tea tree oil.

Another embodiment comprises cyclosporin A, DeThox Acid O-9®, and Tea tree oil.

Another embodiment comprises cyclosporin A, DeThox Acid TO-14®, and Tea tree oil.

Another embodiment comprises cyclosporin A, DeThox Acid TO-16.5®, and Tea tree oil.

Another embodiment comprises cyclosporin A, DeThox Acid TO-8.5®, and Tea tree oil.

Another embodiment comprises cyclosporin A, Dur-Em 114®, and Tea tree oil.

Another embodiment comprises cyclosporin A, Dur-Em 117®, and Tea tree oil.

Another embodiment comprises cyclosporin A, Dur-Em 204®, and Tea tree oil.

Another embodiment comprises cyclosporin A, Dur-Em 207®, and Tea tree oil.

Another embodiment comprises cyclosporin A, Dur-Em 207E®, and Tea tree oil.

Another embodiment comprises cyclosporin A, Emerest 2704®, and Tea tree oil.

Another embodiment comprises cyclosporin A, Emerest 2712®, and Tea tree oil.

Another embodiment comprises cyclosporin A, Emerest 2715®, and Tea tree oil.

Another embodiment comprises cyclosporin A, Ethal EH-2®, and Tea tree oil.

Another embodiment comprises cyclosporin A, Ethal EH-5®, and Tea tree oil.

Another embodiment comprises cyclosporin A, Ethal OA-23®, and Tea tree oil.

Another embodiment comprises cyclosporin A, Ethosperse CA-20®, and Tea tree oil.

Another embodiment comprises cyclosporin A, Ethosperse LA-12®, and Tea tree oil.

Another embodiment comprises cyclosporin A, Ethosperse LA-23®, and Tea tree oil.

Another embodiment comprises cyclosporin A, Ethosperse LA-4®, and Tea tree oil.

Another embodiment comprises cyclosporin A, Ethox 3095®, and Tea tree oil.

Another embodiment comprises cyclosporin A, Ethox CO-16®, and Tea tree oil.

Another embodiment comprises cyclosporin A, Ethox CO-200®, and Tea tree oil.

Another embodiment comprises cyclosporin A, Ethox CO-25®, and Tea tree oil.

Another embodiment comprises cyclosporin A, Ethox CO-30®, and Tea tree oil.

Another embodiment comprises cyclosporin A, Ethox CO-36®, and Tea tree oil.

Another embodiment comprises cyclosporin A, Ethox CO-40®, and Tea tree oil.

Another embodiment comprises cyclosporin A, Ethox CO-5®, and Tea tree oil.

Another embodiment comprises cyclosporin A, Ethox DO-14®, and Tea tree oil.

Another embodiment comprises cyclosporin A, Ethox DO-9®, and Tea tree oil.

Another embodiment comprises cyclosporin A, Ethox MA-8®, and Tea tree oil.

Another embodiment comprises cyclosporin A, Ethox MI-14®, and Tea tree oil.

Another embodiment comprises cyclosporin A, Ethox ML-14®, and Tea tree oil.

Another embodiment comprises cyclosporin A, Ethox ML-5®, and Tea tree oil.

Another embodiment comprises cyclosporin A, Ethox MO-14®, and Tea tree oil.

Another embodiment comprises cyclosporin A, Ethox MO-9®, and Tea tree oil.

Another embodiment comprises cyclosporin A, Ethox MS-14®, and Tea tree oil.

Another embodiment comprises cyclosporin A, Ethox MS-23®, and Tea tree oil.

Another embodiment comprises cyclosporin A, Ethox MS-40®, and Tea tree oil.

Another embodiment comprises cyclosporin A, Ethox MS-8®, and Tea tree oil.

Another embodiment comprises cyclosporin A, Ethox TO-16®, and Tea tree oil.

Another embodiment comprises cyclosporin A, Ethox TO-8®, and Tea tree oil.

Another embodiment comprises cyclosporin A, Ethylan SN-120®, and Tea tree oil.

Another embodiment comprises cyclosporin A, Ethylan SN-70®, and Tea tree oil.

Another embodiment comprises cyclosporin A, Ethylan SN-90®, and Tea tree oil.

Another embodiment comprises cyclosporin A, Flexricin 13®, and Tea tree oil.

Another embodiment comprises cyclosporin A, Flexricin 15®, and Tea tree oil.

Another embodiment comprises cyclosporin A, Genapol 1261®, and Tea tree oil.

Another embodiment comprises cyclosporin A, Generol 122 E25®, and Tea tree oil.

Another embodiment comprises cyclosporin A, Glicepol 160®, and Tea tree oil.

Another embodiment comprises cyclosporin A, Glicepol 560®, and Tea tree oil.

Another embodiment comprises cyclosporin A, Glicepol GMS 20®, and Tea tree oil.

Another embodiment comprises cyclosporin A, Hetoxide BN-13®, and Tea tree oil.

Another embodiment comprises cyclosporin A, Hetoxide DNP-4®, and Tea tree oil.

Another embodiment comprises cyclosporin A, Hetoxide DNP-9.6®, and Tea tree oil.

Another embodiment comprises cyclosporin A, Hetoxide G-26®, and Tea tree oil.

Another embodiment comprises cyclosporin A, Hetoxide G-7®, and Tea tree oil.

Another embodiment comprises cyclosporin A, Hetoxide GT-20®, and Tea tree oil.

Another embodiment comprises cyclosporin A, Hetoxide HC-40®, and Tea tree oil.

Another embodiment comprises cyclosporin A, Hetoxol C-24®, and Tea tree oil.

Another embodiment comprises cyclosporin A, Hetoxol CA-10®, and Tea tree oil.

Another embodiment comprises cyclosporin A, Hetoxol CA-2®, and Tea tree oil.

Another embodiment comprises cyclosporin A, Hetoxol CA-20®, and Tea tree oil.

Another embodiment comprises cyclosporin A, Hetoxol CAWS®, and Tea tree oil.

Another embodiment comprises cyclosporin A, Hetoxol CD-3®, and Tea tree oil.

Another embodiment comprises cyclosporin A, Hetoxol CD-4®, and Tea tree oil.

Another embodiment comprises cyclosporin A, Hetoxol CS-15®, and Tea tree oil.

Another embodiment comprises cyclosporin A, Hetoxol CS-20®, and Tea tree oil.

Another embodiment comprises cyclosporin A, Hetoxol CS-20D®, and Tea tree oil.

Another embodiment comprises cyclosporin A, Hetoxol CS-25®, and Tea tree oil.

Another embodiment comprises cyclosporin A, Hetoxol CS-50®, and Tea tree oil.

Another embodiment comprises cyclosporin A, Hetoxol CS-9®, and Tea tree oil.

Another embodiment comprises cyclosporin A, Hetoxol CSA-15®, and Tea tree oil.

Another embodiment comprises cyclosporin A, Hetoxol D®, and Tea tree oil.

Another embodiment comprises cyclosporin A, Hetoxol G®, and Tea tree oil.

Another embodiment comprises cyclosporin A, Hetoxol J®, and Tea tree oil.

Another embodiment comprises cyclosporin A, Hetoxol L®, and Tea tree oil.

Another embodiment comprises cyclosporin A, Hetoxol L-23®, and Tea tree oil.

Another embodiment comprises cyclosporin A, Hetoxol L-4®, and Tea tree oil.

Another embodiment comprises cyclosporin A, Hetoxol L-9®, and Tea tree oil.

Another embodiment comprises cyclosporin A, Hetoxol LS-9®, and Tea tree oil.

Another embodiment comprises cyclosporin A, Hetoxol M-3®, and Tea tree oil.

Another embodiment comprises cyclosporin A, Hetoxol OA-10 Special®, and Tea tree oil.

Another embodiment comprises cyclosporin A, Hetoxol OA-20 Special®, and Tea tree oil.

Another embodiment comprises cyclosporin A, Hetoxol OA-3 Special®, and Tea tree oil.

Another embodiment comprises cyclosporin A, Hetoxol OA-5 Special®, and Tea tree oil.

Another embodiment comprises cyclosporin A, Hetoxol OL-10®, and Tea tree oil.

Another embodiment comprises cyclosporin A, Hetoxol OL-2®, and Tea tree oil.

Another embodiment comprises cyclosporin A, Hetoxol OL-23®, and Tea tree oil.

Another embodiment comprises cyclosporin A, Hetoxol OL-4®, and Tea tree oil.

Another embodiment comprises cyclosporin A, Hetoxol OL-40®, and Tea tree oil.

Another embodiment comprises cyclosporin A, Hetoxol OL-5®, and Tea tree oil.

Another embodiment comprises cyclosporin A, Hetoxol PLA®, and Tea tree oil.

Another embodiment comprises cyclosporin A, Hetoxol SP-15®, and Tea tree oil.

Another embodiment comprises cyclosporin A, Hetoxol STA-10®, and Tea tree oil.

Another embodiment comprises cyclosporin A, Hetoxol STA-2®, and Tea tree oil.

Another embodiment comprises cyclosporin A, Hetoxol STA-30®, and Tea tree oil.

Another embodiment comprises cyclosporin A, Hetoxol TD-12®, and Tea tree oil.

Another embodiment comprises cyclosporin A, Hetoxol TD-18®, and Tea tree oil.

Another embodiment comprises cyclosporin A, Hetoxol TD-3®, and Tea tree oil.

Another embodiment comprises cyclosporin A, Hetoxol TD-6®, and Tea tree oil.

Another embodiment comprises cyclosporin A, Hetoxol TD-9®, and Tea tree oil.

Another embodiment comprises cyclosporin A, Hetoxol TDEP-15®, and Tea tree oil.

Another embodiment comprises cyclosporin A, Hetoxol TDEP-63®, and Tea tree oil.

Another embodiment comprises cyclosporin A, Iconol 24-12®, and Tea tree oil.

Another embodiment comprises cyclosporin A, Iconol 24-23®, and Tea tree oil.

Another embodiment comprises cyclosporin A, Iconol 24-4®, and Tea tree oil.

Another embodiment comprises cyclosporin A, Iconol DA-4®, and Tea tree oil.

Another embodiment comprises cyclosporin A, Iconol DA-6®, and Tea tree oil.

Another embodiment comprises cyclosporin A, Iconol TDA-10®, and Tea tree oil.

Another embodiment comprises cyclosporin A, Iconol TDA-3®, and Tea tree oil.

Another embodiment comprises cyclosporin A, Iconol TDA-6®, and Tea tree oil.

Another embodiment comprises cyclosporin A, Iconol TDA-8-90%®, and Tea tree oil.

Another embodiment comprises cyclosporin A, Iconol TDA-9®, and Tea tree oil.

Another embodiment comprises cyclosporin A, Igepal CA-210®, and Tea tree oil.

Another embodiment comprises cyclosporin A, Igepal CA-407®, and Tea tree oil.

Another embodiment comprises cyclosporin A, Igepal CA-420®, and Tea tree oil.

Another embodiment comprises cyclosporin A, Igepal CA-520®, and Tea tree oil.

Another embodiment comprises cyclosporin A, Igepal CA-620®, and Tea tree oil.

Another embodiment comprises cyclosporin A, Igepal CA-630®, and Tea tree oil.

Another embodiment comprises cyclosporin A, Igepal CA-720®, and Tea tree oil.

Another embodiment comprises cyclosporin A, Igepal CA-877®, and Tea tree oil.

Another embodiment comprises cyclosporin A, Igepal CA-887®, and Tea tree oil.

Another embodiment comprises cyclosporin A, Igepal CA-897®, and Tea tree oil.

Another embodiment comprises cyclosporin A, Igepal CO-210®, and Tea tree oil.

Another embodiment comprises cyclosporin A, Igepal CO-430®, and Tea tree oil.

Another embodiment comprises cyclosporin A, Igepal CO-520®, and Tea tree oil.

Another embodiment comprises cyclosporin A, Igepal CO-530®, and Tea tree oil.

Another embodiment comprises cyclosporin A, Igepal CO-610®, and Tea tree oil.

Another embodiment comprises cyclosporin A, Igepal CO-630®, and Tea tree oil.

Another embodiment comprises cyclosporin A, Igepal CO-630 Special®, and Tea tree oil.

Another embodiment comprises cyclosporin A, Igepal CO-660®, and Tea tree oil.

Another embodiment comprises cyclosporin A, Igepal CO-710®, and Tea tree oil.

Another embodiment comprises cyclosporin A, Igepal CO-720®, and Tea tree oil.

Another embodiment comprises cyclosporin A, Igepal CO-730®, and Tea tree oil.

Another embodiment comprises cyclosporin A, Igepal CO-738®, and Tea tree oil.

Another embodiment comprises cyclosporin A, Igepal CO-850®, and Tea tree oil.

Another embodiment comprises cyclosporin A, Igepal CO-880®, and Tea tree oil.

Another embodiment comprises cyclosporin A, Igepal CO-887®, and Tea tree oil.

Another embodiment comprises cyclosporin A, Igepal CO-890®, and Tea tree oil.

Another embodiment comprises cyclosporin A, Igepal CO-897®, and Tea tree oil.

Another embodiment comprises cyclosporin A, Igepal CO-970®, and Tea tree oil.

Another embodiment comprises cyclosporin A, Igepal CO-977®, and Tea tree oil.

Another embodiment comprises cyclosporin A, Igepal CO-987®, and Tea tree oil.

Another embodiment comprises cyclosporin A, Igepal CO-990 FLK®, and Tea tree oil.

Another embodiment comprises cyclosporin A, Igepal CO-997®, and Tea tree oil.

Another embodiment comprises cyclosporin A, Igepal DM-430®, and Tea tree oil.

Another embodiment comprises cyclosporin A, Igepal DM-530®, and Tea tree oil.

Another embodiment comprises cyclosporin A, Igepal DM-710®, and Tea tree oil.

Another embodiment comprises cyclosporin A, lgepal DM-730®, and Tea tree oil.

Another embodiment comprises cyclosporin A, Igepal DM-880®, and Tea tree oil.

Another embodiment comprises cyclosporin A, Igepal DM-970 FLK®, and Tea tree oil.

Another embodiment comprises cyclosporin A, Igepal OD-410®, and Tea tree oil.

Another embodiment comprises cyclosporin A, Igepal RC-520®, and Tea tree oil.

Another embodiment comprises cyclosporin A, Igepal RC-620®, and Tea tree oil.

Another embodiment comprises cyclosporin A, Igepal RC-630®, and Tea tree oil.

Another embodiment comprises cyclosporin A, Lexemul CS-20®, and Tea tree oil.

Another embodiment comprises cyclosporin A, Lipo GMS 450®, and Tea tree oil.

Another embodiment comprises cyclosporin A, Lipo Polyglycol 1000®, and Tea tree oil.

Another embodiment comprises cyclosporin A, Lipo Polyglycol 200®, and Tea tree oil.

Another embodiment comprises cyclosporin A, Lipo Polyglycol 300®, and Tea tree oil.

Another embodiment comprises cyclosporin A, Lipo Polyglycol 3350®, and Tea tree oil.

Another embodiment comprises cyclosporin A, Lipo Polyglycol 400®, and Tea tree oil.

Another embodiment comprises cyclosporin A, Lipo Polyglycol 600®, and Tea tree oil.

Another embodiment comprises cyclosporin A, Lipocol C-10®, and Tea tree oil.

Another embodiment comprises cyclosporin A, Lipocol C-2®, and Tea tree oil.

Another embodiment comprises cyclosporin A, Lipocol C-20®, and Tea tree oil.

Another embodiment comprises cyclosporin A, Lipocol L-12®, and Tea tree oil.

Another embodiment comprises cyclosporin A, Lipocol L-23®, and Tea tree oil.

Another embodiment comprises cyclosporin A, Lipocol L-4®, and Tea tree oil.

Another embodiment comprises cyclosporin A, Lipocol O-10®, and Tea tree oil.

Another embodiment comprises cyclosporin A, Lipocol O-2®, and Tea tree oil.

Another embodiment comprises cyclosporin A, Lipocol O-20®, and Tea tree oil.

Another embodiment comprises cyclosporin A, Lipocol O-5®, and Tea tree oil.

Another embodiment comprises cyclosporin A, Lipocol S-10®, and Tea tree oil.

Another embodiment comprises cyclosporin A, Lipocol S-2®, and Tea tree oil.

Another embodiment comprises cyclosporin A, Lipocol S-20®, and Tea tree oil.

Another embodiment comprises cyclosporin A, Lipocol SC-15®, and Tea tree oil.

Another embodiment comprises cyclosporin A, Lipocol SC-20®, and Tea tree oil.

Another embodiment comprises cyclosporin A, Lipocol SC-4®, and Tea tree oil.

Another embodiment comprises cyclosporin A, Lipocol TD-12®, and Tea tree oil.

Another embodiment comprises cyclosporin A, Lumulse 40-O K®, and Tea tree oil.

Another embodiment comprises cyclosporin A, Lumulse 42-O K®, and Tea tree oil.

Another embodiment comprises cyclosporin A, Lumulse GMT-40®, and Tea tree oil.

Another embodiment comprises cyclosporin A, Lumulse PEG 1450®, and Tea tree oil.

Another embodiment comprises cyclosporin A, Lumulse PEG 1450 NF®, and Tea tree oil.

Another embodiment comprises cyclosporin A, Lumulse PEG 8000®, and Tea tree oil.

Another embodiment comprises cyclosporin A, Lumulse PGO®, and Tea tree oil.

Another embodiment comprises cyclosporin A, Lumulse POE (2) Oleyl Amine®, and Tea tree oil.

Another embodiment comprises cyclosporin A, Magrabar PGO-104®, and Tea tree oil.

Another embodiment comprises cyclosporin A, Mapeg 200 ML®, and Tea tree oil.

Another embodiment comprises cyclosporin A, Mapeg 400 MO®, and Tea tree oil.

Another embodiment comprises cyclosporin A, Mapeg 400 MOT®, and Tea tree oil.

Another embodiment comprises cyclosporin A, Mapeg 400DO®, and Tea tree oil.

Another embodiment comprises cyclosporin A, Mapeg 400DOT®, and Tea tree oil.

Another embodiment comprises cyclosporin A, Mapeg 600DOT®, and Tea tree oil.

Another embodiment comprises cyclosporin A, Marlox B 24/80®, and Tea tree oil.

Another embodiment comprises cyclosporin A, Marlox FK 86®, and Tea tree oil.

Another embodiment comprises cyclosporin A, Marlox MO 15®, and Tea tree oil.

Another embodiment comprises cyclosporin A, Mazox CDA CG®, and Tea tree oil.

Another embodiment comprises cyclosporin A, Monoglycerides Glyceryl Monestearate®, and Tea tree oil.

Another embodiment comprises cyclosporin A, Myrj 45®, and Tea tree oil.

Another embodiment comprises cyclosporin A, Myrj 52®, and Tea tree oil.

Another embodiment comprises cyclosporin A, Myrj 52S®, and Tea tree oil.

Another embodiment comprises cyclosporin A, Myrj 53®, and Tea tree oil.

Another embodiment comprises cyclosporin A, Myrj 59®, and Tea tree oil.

Another embodiment comprises cyclosporin A, Norfox NP-12®, and Tea tree oil.

Another embodiment comprises cyclosporin A, Norfox NP-4®, and Tea tree oil.

Another embodiment comprises cyclosporin A, Norfox NP-6®, and Tea tree oil.

Another embodiment comprises cyclosporin A, Norfox NP-887®, and Tea tree oil.

Another embodiment comprises cyclosporin A, Norfox NP-9®, and Tea tree oil.

Another embodiment comprises cyclosporin A, Norfox NP-977®, and Tea tree oil.

Another embodiment comprises cyclosporin A, Norfox OP-100®, and Tea tree oil.

Another embodiment comprises cyclosporin A, Norfox OP-102®, and Tea tree oil.

Another embodiment comprises cyclosporin A, Norfox OP-114®, and Tea tree oil.

Another embodiment comprises cyclosporin A, Norfox OP-45®, and Tea tree oil.

Another embodiment comprises cyclosporin A, Pegosperse 100-L¢, and Tea tree oil.

Another embodiment comprises cyclosporin A, Pegosperse 100-O®, and Tea tree oil.

Another embodiment comprises cyclosporin A, Pegosperse 100-S®, and Tea tree oil.

Another embodiment comprises cyclosporin A, Pegosperse 1500-MS®, and Tea tree oil.

Another embodiment comprises cyclosporin A, Pegosperse 1750-MS®, and Tea tree oil.

Another embodiment comprises cyclosporin A, Pegosperse 200 DL®, and Tea tree oil.

Another embodiment comprises cyclosporin A, Pegosperse 200-ML®, and Tea tree oil.

Another embodiment comprises cyclosporin A, Pegosperse 400-DL®, and Tea tree oil.

Another embodiment comprises cyclosporin A, Pegosperse 400-DO®, and Tea tree oil.

Another embodiment comprises cyclosporin A, Pegosperse 400-DS®, and Tea tree oil.

Another embodiment comprises cyclosporin A, Pegosperse 400-ML®, and Tea tree oil.

Another embodiment comprises cyclosporin A, Pegosperse 400-MO®, and Tea tree oil.

Another embodiment comprises cyclosporin A, Pegosperse 400-MOT®, and Tea tree oil.

Another embodiment comprises cyclosporin A, Pegosperse 400-MS®, and Tea tree oil.

Another embodiment comprises cyclosporin A, Pegosperse 50-DS®, and Tea tree oil.

Another embodiment comprises cyclosporin A, Pegosperse 50-MS®, and Tea tree oil.

Another embodiment comprises cyclosporin A, Pegosperse 600-ML®, and Tea tree oil.

Another embodiment comprises cyclosporin A, Pegosperse 600-MS®, and Tea tree oil.

Another embodiment comprises cyclosporin A, Plurafac A-38®, and Tea tree oil.

Another embodiment comprises cyclosporin A, Plurafac B-25-50®, and Tea tree oil.

Another embodiment comprises cyclosporin A, Plurafac B-26®, and Tea tree oil.

Another embodiment comprises cyclosporin A, Plurafac D-25®, and Tea tree oil.

Another embodiment comprises cyclosporin A, Plurafac LF 131®, and Tea tree oil.

Another embodiment comprises cyclosporin A, Plurafac LF 4030®, and Tea tree oil.

Another embodiment comprises cyclosporin A, Plurafac LF 7000®, and Tea tree oil.

Another embodiment comprises cyclosporin A, Plurafac RA-20®, and Tea tree oil.

Another embodiment comprises cyclosporin A, Plurafac RA-30®, and Tea tree oil.

Another embodiment comprises cyclosporin A, Plurafac RA-40®, and Tea tree oil.

Another embodiment comprises cyclosporin A, Pluronic 10R5®, and Tea tree oil.

Another embodiment comprises cyclosporin A, Pluronic 17R2®, and Tea tree oil.

Another embodiment comprises cyclosporin A, Pluronic 17R4®, and Tea tree oil.

Another embodiment comprises cyclosporin A, Pluronic 25R2®, and Tea tree oil.

Another embodiment comprises cyclosporin A, Pluronic 25R4®, and Tea tree oil.

Another embodiment comprises cyclosporin A, Pluronic 31R1®, and Tea tree oil.

Another embodiment comprises cyclosporin A, Pluronic F108®, and Tea tree oil.

Another embodiment comprises cyclosporin A, Pluronic F127®, and Tea tree oil.

Another embodiment comprises cyclosporin A, Pluronic F38®, and Tea tree oil.

Another embodiment comprises cyclosporin A, Pluronic F68®, and Tea tree oil.

Another embodiment comprises cyclosporin A, Pluronic F68LF®, and Tea tree oil.

Another embodiment comprises cyclosporin A, Pluronic F77®, and Tea tree oil.

Another embodiment comprises cyclosporin A, Pluronic F87®, and Tea tree oil.

Another embodiment comprises cyclosporin A, Pluronic F88®, and Tea tree oil.

Another embodiment comprises cyclosporin A, Pluronic F98®, and Tea tree oil.

Another embodiment comprises cyclosporin A, Pluronic L10®, and Tea tree oil.

Another embodiment comprises cyclosporin A, Pluronic L101®, and Tea tree oil.

Another embodiment comprises cyclosporin A, Pluronic L121®, and Tea tree oil.

Another embodiment comprises cyclosporin A, Pluronic L31®, and Tea tree oil.

Another embodiment comprises cyclosporin A, Pluronic L35®, and Tea tree oil.

Another embodiment comprises cyclosporin A, Pluronic L43®, and Tea tree oil.

Another embodiment comprises cyclosporin A, Pluronic L44®, and Tea tree oil.

Another embodiment comprises cyclosporin A, Pluronic L61®, and Tea tree oil.

Another embodiment comprises cyclosporin A, Pluronic L62®, and Tea tree oil.

Another embodiment comprises cyclosporin A, Pluronic L62D®, and Tea tree oil.

Another embodiment comprises cyclosporin A, Pluronic L62LF®, and Tea tree oil.

Another embodiment comprises cyclosporin A, Pluronic L64®, and Tea tree oil.

Another embodiment comprises cyclosporin A, Pluronic L81®, and Tea tree oil.

Another embodiment comprises cyclosporin A, Pluronic L92®, and Tea tree oil.

Another embodiment comprises cyclosporin A, Pluronic P103®, and Tea tree oil.

Another embodiment comprises cyclosporin A, Pluronic P104®, and Tea tree oil.

Another embodiment comprises cyclosporin A, Pluronic P105®, and Tea tree oil.

Another embodiment comprises cyclosporin A, Pluronic P123®, and Tea tree oil.

Another embodiment comprises cyclosporin A, Pluronic P65®, and Tea tree oil.

Another embodiment comprises cyclosporin A, Pluronic P84®, and Tea tree oil.

Another embodiment comprises cyclosporin A, Pluronic P85®, and Tea tree oil.

Another embodiment comprises cyclosporin A, Polyaldo 10-2-P®, and Tea tree oil.

Another embodiment comprises cyclosporin A, Polyaldo DGDO®, and Tea tree oil.

Another embodiment comprises cyclosporin A, Polyaldo HGDS®, and Tea tree oil.

Another embodiment comprises cyclosporin A, Polyaldo TGMS®, and Tea tree oil.

Another embodiment comprises cyclosporin A, Protegin V®, and Tea tree oil.

Another embodiment comprises cyclosporin A, Protegin W®, and Tea tree oil.

Another embodiment comprises cyclosporin A, Protegin WX®, and Tea tree oil.

Another embodiment comprises cyclosporin A, Protegin XV®, and Tea tree oil.

Another embodiment comprises cyclosporin A, Rhodasurf BC-420®, and Tea tree oil.

Another embodiment comprises cyclosporin A, Rhodasurf BC-610®, and Tea tree oil.

Another embodiment comprises cyclosporin A, Rhodasurf BC-630®, and Tea tree oil.

Another embodiment comprises cyclosporin A, Rhodasurf BC-720®, and Tea tree oil.

Another embodiment comprises cyclosporin A, Rhodasurf BC-840®, and Tea tree oil.

Another embodiment comprises cyclosporin A, Rhodasurf L 1®, and Tea tree oil.

Another embodiment comprises cyclosporin A, Rhodasurf L 12®, and Tea tree oil.

Another embodiment comprises cyclosporin A, Rhodasurf L 2®, and Tea tree oil.

Another embodiment comprises cyclosporin A, Rhodasurf L 20®, and Tea tree oil.

Another embodiment comprises cyclosporin A, Rhodasurf L 3®, and Tea tree oil.

Another embodiment comprises cyclosporin A, Rhodasurf L 30®, and Tea tree oil.

Another embodiment comprises cyclosporin A, Rhodasurf L 4®, and Tea tree oil.

Another embodiment comprises cyclosporin A, Rhodasurf L 50®, and tea tree oil.

Another embodiment comprises cyclosporin A, Rhodasurf L 7/90®, and Tea tree oil.

Another embodiment comprises cyclosporin A, Rhodasurf LA-3®, and Tea tree oil.

Another embodiment comprises cyclosporin A, Rhodasurf LA-7®, and Tea tree oil.

Another embodiment comprises cyclosporin A, Rhodasurf LAN-23®, and Tea tree oil.

Another embodiment comprises cyclosporin A, Rhodasurf ON-870®, and Tea tree oil.

Another embodiment comprises cyclosporin A, Rhodasurf ON-877®, and Tea tree oil.

Another embodiment comprises cyclosporin A, Rhodasurf TDA-8/5®, and Tea tree oil.

Another embodiment comprises cyclosporin A, Ritapro 100®, and Tea tree oil.

Another embodiment comprises cyclosporin A, Ritapro 165®, and Tea tree oil.

Another embodiment comprises cyclosporin A, Ritapro 200®, and Tea tree oil.

Another embodiment comprises cyclosporin A, Ritapro 300®, and Tea tree oil.

Another embodiment comprises cyclosporin A, Ritoleth 10®, and Tea tree oil.

Another embodiment comprises cyclosporin A, Ritoleth 2®, and Tea tree oil.

Another embodiment comprises cyclosporin A, Ritoleth 20®, and Tea tree oil.

Another embodiment comprises cyclosporin A, Ritoleth 5®, and Tea tree oil.

Another embodiment comprises cyclosporin A, Santone 10-10-0®, and Tea tree oil.

Another embodiment comprises cyclosporin A, Santone 3-1-S XTR®, and Tea tree oil.

Another embodiment comprises cyclosporin A, Santone 3-1-SH®, and Tea tree oil.

Another embodiment comprises cyclosporin A, Santone 8-1-O®, and Tea tree oil.

Another embodiment comprises cyclosporin A, Solulan 16®, and Tea tree oil.

Another embodiment comprises cyclosporin A, Solulan 75®, and Tea tree oil.

Another embodiment comprises cyclosporin A, Solulan C-24®, and Tea tree oil.

Another embodiment comprises cyclosporin A, Solulan L-575®, and Tea tree oil.

Another embodiment comprises cyclosporin A, Standamox CAW®, and Tea tree oil.

Another embodiment comprises cyclosporin A, Surfine AZI-A®, and Tea tree oil.

Another embodiment comprises cyclosporin A, Surfonic CO-15®, and Tea tree oil.

Another embodiment comprises cyclosporin A, Surfonic CO-25®, and Tea tree oil.

Another embodiment comprises cyclosporin A, Surfonic CO-30®, and Tea tree oil.

Another embodiment comprises cyclosporin A, Surfonic CO-36®, and Tea tree oil.

Another embodiment comprises cyclosporin A, Surfonic CO-42®, and Tea tree oil.

Another embodiment comprises cyclosporin A, Surfonic E400-MO®, and Tea tree oil.

Another embodiment comprises cyclosporin A, Surfonic L12-3®, and Tea tree oil.

Another embodiment comprises cyclosporin A, Surfonic L12-6®, and Tea tree oil.

Another embodiment comprises cyclosporin A, Surfonic L12-8®, and Tea tree oil.

Another embodiment comprises cyclosporin A, Surfonic L24-12®, and Tea tree oil.

Another embodiment comprises cyclosporin A, Surfonic L24-2®, and Tea tree oil.

Another embodiment comprises cyclosporin A, Surfonic L24-22®, and Tea tree oil.

Another embodiment comprises cyclosporin A, Surfonic L24-3®, and Tea tree oil.

Another embodiment comprises cyclosporin A, Surfonic L24-4®, and Tea tree oil.

Another embodiment comprises cyclosporin A, Surfonic L24-5®, and Tea tree oil.

Another embodiment comprises cyclosporin A, Surfonic L24-7®, and Tea tree oil.

Another embodiment comprises cyclosporin A, Surfonic L24-7.1®, and Tea tree oil.

Another embodiment comprises cyclosporin A, Surfonic L24-9®, and Tea tree oil.

Another embodiment comprises cyclosporin A, Surfonic LF-17®, and Tea tree oil.

Another embodiment comprises cyclosporin A, Surfonic LF-18®, and Tea tree oil.

Another embodiment comprises cyclosporin A, Surfonic LF-37®, and Tea tree oil.

Another embodiment comprises cyclosporin A, Surfonic LF-41®, and Tea tree oil.

Another embodiment comprises cyclosporin A, Surfonic LF-42®, and Tea tree oil.

Another embodiment comprises cyclosporin A, Surfonic LF-47®, and Tea tree oil.

Another embodiment comprises cyclosporin A, Surfonic LF-68®, and Tea tree oil.

Another embodiment comprises cyclosporin A, Surfonic N-10®, and Tea tree oil.

Another embodiment comprises cyclosporin A, Surfonic N-100®, and Tea tree oil.

Another embodiment comprises cyclosporin A, Surfonic N-102®, and Tea tree oil.

Another embodiment comprises cyclosporin A, Surfonic N-120®, and Tea tree oil.

Another embodiment comprises cyclosporin A, Surfonic N-150®, and Tea tree oil.

Another embodiment comprises cyclosporin A, Surfonic N-200®, and Tea tree oil.

Another embodiment comprises cyclosporin A, Surfonic N-300®, and Tea tree oil.

Another embodiment comprises cyclosporin A, Surfonic N-31.5®, and Tea tree oil.

Another embodiment comprises cyclosporin A, Surfonic N-40®, and Tea tree oil.

Another embodiment comprises cyclosporin A, Surfonic N-400®, and Tea tree oil.

Another embodiment comprises cyclosporin A, Surfonic N-60(®, and Tea tree oil.

Another embodiment comprises cyclosporin A, Surfonic N-85®, and Tea tree oil.

Another embodiment comprises cyclosporin A, Surfonic N-95®, and Tea tree oil.

Another embodiment comprises cyclosporin A, Surfonic NB-18®, and Tea tree oil.

Another embodiment comprises cyclosporin A, Surfonic NB-23®, and Tea tree oil.

Another embodiment comprises cyclosporin A, Surfonic NB-25®, and Tea tree oil.

Another embodiment comprises cyclosporin A, Surfonic NB-307®, and Tea tree oil.

Another embodiment comprises cyclosporin A, Surfonic NB-31®, and Tea tree oil.

Another embodiment comprises cyclosporin A, Surfonic NB-41®, and Tea tree oil.

Another embodiment comprises cyclosporin A, Surfonic NB-43®, and Tea tree oil.

Another embodiment comprises cyclosporin A, Surfonic OP-100®, and Tea tree oil.

Another embodiment comprises cyclosporin A, Surfonic OP-120®, and Tea tree oil.

Another embodiment comprises cyclosporin A, Surfonic OP-15®, and Tea tree oil.

Another embodiment comprises cyclosporin A, Surfonic OP-35®, and Tea tree oil.

Another embodiment comprises cyclosporin A, Surfonic OP-70®, and Tea tree oil.

Another embodiment comprises cyclosporin A, Surfonic P1®, and Tea tree oil.

Another embodiment comprises cyclosporin A, Surfonic P3®, and Tea tree oil.

Another embodiment comprises cyclosporin A, Surfonic P5®, and Tea tree oil.

Another embodiment comprises cyclosporin A, Surfonic P6®, and Tea tree oil.

Another embodiment comprises cyclosporin A, Surfonic POA-17R2®, and Tea tree oil.

Another embodiment comprises cyclosporin A, Surfonic POA-25R2®, and Tea tree oil.

Another embodiment comprises cyclosporin A, Surfonic TDA-11®, and Tea tree oil.

Another embodiment comprises cyclosporin A, Surfonic TDA-3B®, and Tea tree oil.

Another embodiment comprises cyclosporin A, Surfonic TDA-6®, and Tea tree oil.

Another embodiment comprises cyclosporin A, Surfonic TDA-6/88®, and Tea tree oil.

Another embodiment comprises cyclosporin A, Surfonic TDA-8®, and Tea tree oil.

Another embodiment comprises cyclosporin A, Surfonic TDA-8/90®, and Tea tree oil.

Another embodiment comprises cyclosporin A, Surfonic TDA-9®, and Tea tree oil.

Another embodiment comprises cyclosporin A, Surfonic TX-CCN®, and Tea tree oil.

Another embodiment comprises cyclosporin A, Tagat CH 40®, and Tea tree oil.

Another embodiment comprises cyclosporin A, Tagat CH 60®, and Tea tree oil.

Another embodiment comprises cyclosporin A, Tagat L 2®, and Tea tree oil.

Another embodiment comprises cyclosporin A, Tagat S®, and Tea tree oil.

Another embodiment comprises cyclosporin A, Tagat S 2®, and Tea tree oil.

Another embodiment comprises cyclosporin A, Tally 100 Plus®, and Tea tree oil.

Another embodiment comprises cyclosporin A, T-Det A106®, and Tea tree oil.

Another embodiment comprises cyclosporin A, T-Det A109®, and Tea tree oil.

Another embodiment comprises cyclosporin A, T-Det A136®, and Tea tree oil.

Another embodiment comprises cyclosporin A, T-Det A139®, and Tea tree oil.

Another embodiment comprises cyclosporin A, T-Det A2412®, and Tea tree oil.

Another embodiment comprises cyclosporin A, T-Det A243®, and Tea tree oil.

Another embodiment comprises cyclosporin A, T-Det A247®, and Tea tree oil.

Another embodiment comprises cyclosporin A, T-Det A249®, and Tea tree oil.

Another embodiment comprises cyclosporin A, T-Det A467®, and Tea tree oil.

Another embodiment comprises cyclosporin A, T-Det COE®, and Tea tree oil.

Another embodiment comprises cyclosporin A, T-Det DD-10®, and Tea tree oil.

Another embodiment comprises cyclosporin A, T-Det DD-5®, and Tea tree oil.

Another embodiment comprises cyclosporin A, T-Det DD-7®, and Tea tree oil.

Another embodiment comprises cyclosporin A, T-Det EPO-104®, and Tea tree oil.

Another embodiment comprises cyclosporin A, T-Det EPO-61®, and Tea tree oil.

Another embodiment comprises cyclosporin A, T-Det EPO-62®, and Tea tree oil.

Another embodiment comprises cyclosporin A, T-Det EPO-64®, and Tea tree oil.

Another embodiment comprises cyclosporin A, T-Det N-1.5®, and Tea tree oil.

Another embodiment comprises cyclosporin A, T-Det N-10.5®, and Tea tree oil.

Another embodiment comprises cyclosporin A, T-Det N-100®, and Tea tree oil.

Another embodiment comprises cyclosporin A, T-Det N-1007®, and Tea tree oil.

Another embodiment comprises cyclosporin A, T-Det N-12®, and Tea tree oil.

Another embodiment comprises cyclosporin A, T-Det N-14®, and Tea tree-oil.

Another embodiment comprises cyclosporin A, T-Det N-20®, and Tea tree oil.

Another embodiment comprises cyclosporin A, T-Det N-30®, and Tea tree oil.

Another embodiment comprises cyclosporin A, T-Det N-307®, and Tea tree oil.

Another embodiment comprises cyclosporin A, T-Det N-4®, and Tea tree oil.

Another embodiment comprises cyclosporin A, T-Det N-40®, and Tea tree oil.

Another embodiment comprises cyclosporin A, T-Det N-407®, and Tea tree oil.

Another embodiment comprises cyclosporin A, T-Det N-50®, and Tea tree oil.

Another embodiment comprises cyclosporin A, T-Det N-507®, and Tea tree oil.

Another embodiment comprises cyclosporin A, T-Det N-6®, and Tea tree oil.

Another embodiment comprises cyclosporin A, T-Det N-70®, and Tea tree oil.

Another embodiment comprises cyclosporin A, T-Det N-8®, and Tea tree oil.

Another embodiment comprises cyclosporin A, T-Det N-9.5®, and Tea tree oil.

Another embodiment comprises cyclosporin A, T-Det O-12®, and Tea tree oil.

Another embodiment comprises cyclosporin A, T-Det O-165®, and Tea tree oil.

Another embodiment comprises cyclosporin A, T-Det O-307®, and Tea tree oil.

Another embodiment comprises cyclosporin A, T-Det O-40®, and Tea tree oil.

Another embodiment comprises cyclosporin A, T-Det O-407®, and Tea tree oil.

Another embodiment comprises cyclosporin A, T-Det O-6®, and Tea tree oil.

Another embodiment comprises cyclosporin A, T-Det O-8®, and Tea tree oil.

Another embodiment comprises cyclosporin A, T-Det O-9®, and Tea tree oil.

Another embodiment comprises cyclosporin A, Tegin®, and Tea tree oil.

Another embodiment comprises cyclosporin A, Tergitol 15-S-12®, and Tea tree oil.

Another embodiment comprises cyclosporin A, Tergitol 15-S-15®, and Tea tree oil.

Another embodiment comprises cyclosporin A, Tergitol 15-S-20®, and Tea tree oil.

Another embodiment comprises cyclosporin A, Tergitol 15-S-20 (80AQ)®, and Tea tree oil.

Another embodiment comprises cyclosporin A, Tergitol 15-S-3®, and Tea tree oil.

Another embodiment comprises cyclosporin A, Tergitol 15-S-30®, and Tea tree oil.

Another embodiment comprises cyclosporin A, Tergitol 15-S-40®, and Tea tree oil.

Another embodiment comprises cyclosporin A, Tergitol 15-S-40 (70AQ)®, and Tea tree oil.

Another embodiment comprises cyclosporin A, Tergitol 15-S-5®, and Tea tree oil.

Another embodiment comprises cyclosporin A, Tergitol 15-S-7®, and Tea tree oil.

Another embodiment comprises cyclosporin A, Tergitol 15-S-9®, and Tea tree oil.

Another embodiment comprises cyclosporin A, Tergitol L-101®, and Tea tree oil.

Another embodiment comprises cyclosporin A, Tergitol L-61®, and Tea tree oil.

Another embodiment comprises cyclosporin A, Tergitol L-62®, and Tea tree oil.

Another embodiment comprises cyclosporin A, Tergitol L-64®, and Tea tree oil.

Another embodiment comprises cyclosporin A, Tergitol L-81®, and Tea tree oil.

Another embodiment comprises cyclosporin A, Tergitol NP-10®, and Tea tree oil.

Another embodiment comprises cyclosporin A, Tergitol NP-11®, and Tea tree oil.

Another embodiment comprises cyclosporin A, Tergitol NP-12®, and Tea tree oil.

Another embodiment comprises cyclosporin A, Tergitol NP-13®, and Tea tree oil.

Another embodiment comprises cyclosporin A, Tergitol NP-15®, and Tea tree oil.

Another embodiment comprises cyclosporin A, Tergitol NP-30®, and Tea tree oil.

Another embodiment comprises cyclosporin A, Tergitol NP-30 (70 AQ)®, and Tea tree oil.

Another embodiment comprises cyclosporin A, Tergitol NP-4®, and Tea tree oil.

Another embodiment comprises cyclosporin A, Tergitol NP-40®, and Tea tree oil.

Another embodiment comprises cyclosporin A, Tergitol NP-40 (70 AQ)®, and Tea tree oil.

Another embodiment comprises cyclosporin A, Tergitol NP-50 (70 AQ)®, and Tea tree oil.

Another embodiment comprises cyclosporin A, Tergitol NP-55 (70 AQ)®, and Tea tree oil.

Another embodiment comprises cyclosporin A, Tergitol NP-6®, and Tea tree oil.

Another embodiment comprises cyclosporin A, Tergitol NP-7®, and Tea tree oil.

Another embodiment comprises cyclosporin A, Tergitol NP-70 (70 AQ)®, and Tea tree oil.

Another embodiment comprises cyclosporin A, Tergitol NP-8®, and Tea tree oil.

Another embodiment comprises cyclosporin A, Tergitol NP-9®, and Tea tree oil.

Another embodiment comprises cyclosporin A, Tergitol NP-9.5®, and Tea tree oil.

Another embodiment comprises cyclosporin A, Tergitol TMN-10®, and Tea tree oil.

Another embodiment comprises cyclosporin A, Tergitol TMN-3®, and Tea tree oil.

Another embodiment comprises cyclosporin A, Tergitol TMN-6®, and Tea tree oil.

Another embodiment comprises cyclosporin A, Tergitol XD®, and Tea tree oil.

Another embodiment comprises cyclosporin A, Tergitol XH®, and Tea tree oil.

Another embodiment comprises cyclosporin A, Tergitol XJ®, and Tea tree oil.

Another embodiment comprises cyclosporin A, Tetronic 1107®, and Tea tree oil.

Another embodiment comprises cyclosporin A, Tetronic 1307®, and Tea tree oil.

Another embodiment comprises cyclosporin A, Tetronic 150R1®, and Tea tree oil.

Another embodiment comprises cyclosporin A, Tetronic 304®, and Tea tree oil.

Another embodiment comprises cyclosporin A, Tetronic 701®, and Tea tree oil.

Another embodiment comprises cyclosporin A, Tetronic 901®, and Tea tree oil.

Another embodiment comprises cyclosporin A, Tetronic 904®, and Tea tree oil.

Another embodiment comprises cyclosporin A, Tetronic 908®, and Tea tree oil.

Another embodiment comprises cyclosporin A, Tetronic 90R4®, and Tea tree oil.

Another embodiment comprises cyclosporin A, Tomadol 1-3®, and Tea tree oil.

Another embodiment comprises cyclosporin A, Tomadol 1-5®, and Tea tree oil.

Another embodiment comprises cyclosporin A, Tomadol 1-7®, and Tea tree oil.

Another embodiment comprises cyclosporin A, Tomadol 23-1®, and Tea tree oil.

Another embodiment comprises cyclosporin A, Tomadol 23-3®, and Tea tree oil.

Another embodiment comprises cyclosporin A, Tomadol 23-5®, and Tea tree oil.

Another embodiment comprises cyclosporin A, Tomadol 23-6.5®, and Tea tree oil.

Another embodiment comprises cyclosporin A, Tomadol 25-12®, and Tea tree oil.

Another embodiment comprises cyclosporin A, Tomadol 25-3®, and Tea tree oil.

Another embodiment comprises cyclosporin A, Tomadol 25-7®, and Tea tree oil.

Another embodiment comprises cyclosporin A, Tomadol 25-9®, and Tea tree oil.

Another embodiment comprises cyclosporin A, Tomadol 45-13®, and Tea tree oil.

Another embodiment comprises cyclosporin A, Tomadol 45-2.5®, and Tea tree oil.

Another embodiment comprises cyclosporin A, Tomadol 45-7®, and Tea tree oil.

Another embodiment comprises cyclosporin A, Tomadol 91-2.5®, and Tea tree oil.

Another embodiment comprises cyclosporin A, Tomadol 91-6®, and Tea tree oil.

Another embodiment comprises cyclosporin A, Tomadol 91-8®, and Tea tree oil.

Another embodiment comprises cyclosporin A, Triton X-100®, and Tea tree oil.

Another embodiment comprises cyclosporin A, Triton X-102®, and Tea tree oil.

Another embodiment comprises cyclosporin A, Triton X-114®, and Tea tree oil.

Another embodiment comprises cyclosporin A, Triton X-15®, and Tea tree oil.

Another embodiment comprises cyclosporin A, Triton X-165 (70%)®, and Tea tree oil.

Another embodiment comprises cyclosporin A, Triton X-207®, and Tea tree oil.

Another embodiment comprises cyclosporin A, Triton X-305 (70%)®, and Tea tree oil.

Another embodiment comprises cyclosporin A, Triton X-35®, and Tea tree oil.

Another embodiment comprises cyclosporin A, Triton X-405 (70%)®, and Tea tree oil.

Another embodiment comprises cyclosporin A, Triton X-45®, and Tea tree oil.

Another embodiment comprises cyclosporin A, Triton X-705 (70%)®, and Tea tree oil.

Another embodiment comprises cyclosporin A, Volpo S-10®, and Tea tree oil.

Another embodiment comprises cyclosporin A, Volpo S-2®, and Tea tree oil.

Another embodiment comprises cyclosporin A, Volpo S-20®, and Tea tree oil.

Another embodiment comprises cyclosporin A, Volpo-10®, and Tea tree oil.

Another embodiment comprises cyclosporin A, Volpo-20®, and Tea tree oil.

Another embodiment comprises cyclosporin A, Volpo-3®, and Tea tree oil.

Another embodiment comprises cyclosporin A, Volpo-5®, and Tea tree oil.

A liquid or emulsion which is ophthalmically acceptable is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid or emulsion should either be packaged for single use, or contain a preservative to prevent contamination over multiple uses.

In accordance with the present invention, the emulsions can be further stabilized using a polyelectrolyte, or polyelectrolytes if more than one, from the family of cross-linked polyacrylates, such as carbomers and Pemulene®.

Pemulen® is a registered trademark of B. F. Goodrich for polymeric emulsifiers and commercially available from B. F. Goodrich Company, Specialty Polymers & Chemicals Division, Cleveland, Ohio. Pemulens are Acrylates/C10-30 Alkyl Acrylate Cross-Polymers. They are high molecular weight co-polymers of acrylic acid and a long chain alkyl methacrylate cross-linked with allyl ethers of pentaerythritol. They contain not less than 52.0 percent and not more than 62.0 percent of carboxylic acid groups. The viscosity of a neutralized 1.0 percent aqueous dispersion is between 9,500 and 26,500 centipoises.

In addition, the tonicity of the emulsions can be further adjusted using glycerine, mannitol, or sorbitol if desired. The pH of the emulsions can be adjusted in a conventional manner using sodium hydroxide to a near physiological pH level and while buffering agents are not required, suitable buffers may include phosphates, citrates, acetates and borates.

Examples of useful formulations are shown below.

| Ingredients | 1 | 2 | 3 | 4 | 5 | 6 | 7 | 8 | 9 | 10 | 11 | 12 | 13 | 14 | 15 |
|---|---|---|---|---|---|---|---|---|---|---|---|---|---|---|---|
| Cyclosporine A | 0.1 | 0.1 | 0.05 | 0.02 | 0.05 | 0.03 | 0.01 | 0.08 | 0.03 | 0.1 | 0.04 | 0.05 | 0.08 | 0.02 | 0.05 |
| Castor oil | 1.00 | 1.2 | | | | | | 0.50 | 0.50 | | | | 0.50 | | |
| Clove oil | 0.70 | | | | 0.50 | 0.40 | 0.20 | | | | 0.50 | 0.80 | | | |
| Cassia oil | | 0.4 | 0.20 | 0.70 | | | | 0.55 | | 0.75 | | | | | |
| Cottonseed oil | | | 0.60 | | | | | | | | | | | | |
| Soybean oil | | | | | | | 0.30 | | | | | | | | |
| Lavender oil | | | | | 0.30 | | | | | 0.90 | | | | | 1.00 |
| Juniper oil | | | | | | 0.20 | | | | | | | | | |
| Corn oil | | | | 0.70 | | | | | | | 0.40 | | | | |
| Olive oil | | | | | | | | 0.35 | | | | | | 1.00 | |
| Mineral Oil | | | | | | | | | | | | | 0.80 | | |
| Polysorbate-80 | 1.00 | 0.80 | | 1.50 | | | 0.30 | | 0.30 | 0.30 | 0.10 | 0.30 | | | 0.60 |
| Diglycerol | 0.70 | | | | | | | 0.20 | | | | | 0.80 | | |
| Polyglycerol-3 | | | 1.00 | | | 0.10 | | | | | 0.80 | | | | |
| Simulsol OX 1005L | | 0.60 | | | | | | | | | | | | | |
| Chemonic 435 | | | | | | | | | | | | | | | |
| Marlowet 4530 | | | | | | | | | | | | | | | |
| Brij52 | | | 0.50 | | 0.70 | 0.60 | | | | | | | | | |
| Brij78 | | | | | | | | 1.20 | | | | | | 0.80 | |
| Bio Soft TD-630 | | | | | | | | | | 1.3 | | | | | |
| Canasol BJ 78 | | | | | 0.50 | | | | | | | | | | |
| Canasol BJ 52 | | | | 0.30 | | | | | | | 0.80 | | | | |
| Rhodasurf BEH 25 | | | | | | | | | | | | | | | |
| Desonic 9D | | | | | | | | | | | | 0.60 | | | 0.40 |
| Glycerin | 2.00 | 1.70 | 1.50 | 2.00 | 1.00 | 2.00 | 2.00 | 2.00 | 2.00 | 2.00 | 1.50 | 2.00 | 2.00 | 1.20 | 2.00 |
| CMC | 0.50 | | | | 0.50 | | | 0.50 | | | | | | 0.50 | |
| Pemulen TR-2 | | 0.05 | 0.03 | 0.05 | | | | | 0.02 | | | 0.02 | | | |
| Purified Water | QS | QS | QS | QS | QS | QS | QS | QS | QS | QS | QS | QS | QS | QS | QS |
| Sodium Hydroxide | pH | pH | pH | pH | pH | pH | pH adj | pH adj | pH adj | pH adj | pH adj | pH adj | pH adj | pH adj | pH adj |
| | adj | adj | adj | adj | adj | adj | | | | | | | | | |
| pH | 7.2 | 7.5 | 6.3 | 7.3 | 7.4 | 7.8 | 6.8 | 7.1 | 6.8 | 7 | 7.2 | 7.2 | 7.4 | 7.6 | 5.9 |

Another useful emulsion consists of the following: 0.1% cyclosporin A, 0.20% castor oil, 0.75% polyethylene oxide 40 stearate (Myrj 52®), 0.2% Polysrobate 80, 1.0% glycerin, 0.6% boric acid, 0.5% carboxymethylcellulose, 100 ppm stabilized oxychloro complex (Purite®), sufficient sodium hydroxide to adjust the pH, and the remainder water, wherein the pH is from 7.3 to 7.5.

Although there has been hereinabove described a particular pharmaceutical composition in the form of a nonirritating emulsion for the purpose of illustrating the manner in which the invention may be used to advantage, it should be appreciated that the invention is not limited thereto. Accordingly, any and all modifications, variations, or equivalent arrangements, which may occur to those skilled in the art, should be considered to be within the scope of the present invention as defined in the appended claims.

1. A composition comprising cyclosporin A, an oil, and a stearate surfactant, wherein said composition is an ophthalmically acceptable emulsion.

2. The composition of claim 1 comprising Almond oil.

3. The composition of claim 1 comprising Corn oil.

4. The composition of claim 1 comprising Arachis oil.

5. The composition of claim 1 comprising Cottonseed oil.

6. The composition of claim 1 comprising Safflower oil.

7. The composition of claim 1 comprising Maize oil.

8. The composition of claim 1 comprising Linseed oil.

9. The composition of claim 1 comprising Rapeseed oil.

10. The composition of claim 1 comprising Soybean oil.

11. The composition of claim 1 comprising Olive oil.

12. The composition of claim 1 comprising Caraway oil.

13. The composition of claim 1 comprising Rosemary oil.

14. The composition of claim 1 comprising Peanut oil.

15. The composition of claim 1 comprising Peppermint oil.

16. The composition of claim 1 comprising Sunflower oil.

17. The composition of claim 1 comprising Eucalyptus oil.

18. The composition of claim 1 comprising Sesame oil.

19. A composition comprising cyclosporin A, an oil having a specific gravity below 0.9, wherein said composition is an ophthalmically acceptable emulsion.

20. The composition of claim 1 comprising Mineral oil.

21. The composition of claim 1 comprising a POE (40) monostearate surfactant.

22. The composition of claim 21 having from about 0.001% to about 0.1% cyclosporin A.

23. The composition of claim 22 having from about 0.005% to about 0.05% cyclosporin A.

24. The composition of claim 23 having about 0.05% cyclosporin A.
